(19) United States
(12) Patent Application Publication
(10) Pub. No.: US 2025/0025443 A1
(43) Pub. Date: Jan. 23, 2025

(54) COMPOUNDS AND METHODS FOR TREATING CORONA VIRUSES

(71) Applicant: CLEAR CREEK BIO, INC., Cambridge, MA (US)

(72) Inventors: John K. DICKSON, Apex, NC (US); Joseph P. VACCA, Telford, PA (US); Maryline KIENLE, Wadenswil (CH); Nadim SHOHDY, Garden City, NY (US); James Francis DEMAREST, Durham, NC (US)

(73) Assignee: CLEAR CREEK BIO, INC., Cambridge, MA (US)

(21) Appl. No.: 18/700,786

(22) PCT Filed: Oct. 13, 2022
(86) PCT No.: PCT/US2022/046607

§ 371 Date: Apr. 12, 2024

Related U.S. Application Data
(60) Provisional application No. 63/255,186, filed on Oct. 13, 2021.
(60) Provisional application No. 63/277,531, filed on Nov. 9, 2021.
(60) Provisional application No. 63/314,357, filed on Feb. 25, 2022.
(60) Provisional application No. 63/352,580, filed on Jun. 15, 2022.

(51) Int. Cl. A61K 31/397; A61K 31/166; A61K 31/277; A61K 31/337; A61K 31/36; A61K 31/381; A61K 31/4025; A61K 31/4045; A61K 31/407; A61K 31/416; A61K 31/4178; A61K 31/4184; A61K 31/426; A61K 31/428; A61K 31/4412; A61K 31/4439; A61K 31/4465; A61K 31/4709; A61K 31/498; A61K 31/4985; A61K 31/506; A61K 31/5377; A61K 31/551; A61P 31/14; C07C 233/65; C07C 243/32; C07C 255/44; C07C 271/16; C07C 311/08; C07D 205/04; C07D 207/20; C07D 209/32; C07D 211/46; C07D 213/74; C07D 215/22; C07D 223/04; C07D 231/18; C07D 231/56; C07D 235/08; C07D 235/26; C07D 239/34; C07D 241/44; C07D 245/02; C07D 277/34; C07D 277/64; C07D 295/073; C07D 305/08; C07D 317/54; C07D 333/20; C07D 333/54; C07D 401/04; C07D 401/12; C07D 403/04; C07D 405/12; C07D 405/14; C07D 409/12; C07D 409/14; C07D 411/14; C07D 413/04; C07D 413/06; C07D 413/14; C07D 471/04; C07D 471/08; C07D 487/04

(52) U.S. Cl. CPC A61K 31/397; A61K 31/166; A61K 31/277; A61K 31/337; A61K 31/36; A61K 31/381; A61K 31/4025; A61K 31/4045; A61K 31/407; A61K 31/416; A61K 31/4178; A61K 31/4184; A61K 31/426; A61K 31/428; A61K 31/4412; A61K 31/4439; A61K 31/4465; A61K 31/4709; A61K 31/498; A61K 31/4985; A61K 31/506; A61K 31/5377; A61K 31/551; A61P 31/14; C07C 233/65; C07C 243/32; C07C 255/44; C07C 271/16; C07C 311/08; C07D 205/04; C07D 207/20; C07D 209/32; C07D 211/46; C07D 213/74; C07D 215/22; C07D 223/04; C07D 231/18; C07D 231/56; C07D 235/08; C07D 235/26; C07D 239/34; C07D 241/44; C07D 245/02; C07D 277/34; C07D 277/64; C07D 295/073; C07D 305/08; C07D 317/54; C07D 333/20; C07D 333/54; C07D 401/04; C07D 401/12; C07D 403/04; C07D 405/12; C07D 405/14; C07D 409/12; C07D 409/14; C07D 411/14; C07D 413/04; C07D 413/06; C07D 413/14; C07D 471/04; C07D 471/08; C07D 487/04

(57) ABSTRACT

This invention provides compounds for the inhibition of papain-like proteases (PLpros) for the inhibition of viruses, including compounds of formula (I″), and pharmaceutically acceptable salts thereof.

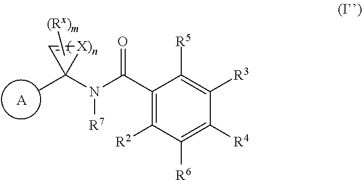

### CROSS-REFERENCE TO RELATED APPLICATIONS

This application claims priority to and the benefit of United States Provisional Patent Application Nos. 63/255,186, filed Oct. 13, 2021; 63/277,531, filed Nov. 9, 2021; 63/314,357, filed Feb. 25, 2022; and 63/352,580, filed Jun. 15, 2022, the contents of each of which are incorporated by reference in their entirety herein.

### FIELD OF THE INVENTION

The invention provides compounds and their methods of use and manufacture for the treatment of hosts infected with coronaviruses, for example, SARS COV-2. The compounds inhibit papain-like cysteine protease (PLpro) and thus can be used to treat any virus that expresses a papain-like cysteine protease (PLpro).

### BACKGROUND

Coronaviruses (CoVs) (order Nidovirales, family Coronaviridae, subfamily Coronavirinae) are enveloped viruses with a positive sense, single-stranded RNA genome. CoVs have a large genome for an RNA virus, ranging in size from 26 to 32 kilobases (kb) in length. The CoV genome encodes four major structural proteins: the spike(S) protein, nucleocapsid (N) protein, membrane (M) protein, and the envelope (E) protein, all of which are required to produce a structurally complete viral particle. See, e.g., P S Masters, The molecular biology of coronavirus. Adv. Virus Res. 2014:101:105-12.

The novel coronavirus, SARS-COV2, is the cause of the current respiratory disease COVID19 which started in Wuhan, China in late 2019. To date, over 400 million people globally have been infected and over 8 million people have died. The pandemic of SARS-COV-2 has caused pressure on the healthcare systems and governments globally.

Given the severe impact of coronaviruses, and notably SARS-COV2, on persons and the healthcare system globally, it is important to provide new treatments for infected people.

It is therefore an object of the present invention to provide new drugs for the treatment of coronaviruses, including SARS-COV2, to treat infected humans or those at risk of infection.

### SUMMARY

This invention provides new compounds and their pharmaceutically acceptable salts, along with their uses and manufacture, for the allosteric inhibition of the papain-like protease (PLpro), a key enzyme used by coronaviruses such as SARS-COV2 that is involved in the generation of a functional replicase complex which enables the virus to replicate. The PLpro also cleaves ubiquitin and ISG15, which are known regulators of host innate immune pathways, and therefore, these allosteric inhibitors can be used to modulate certain immune pathways. Therefore, in certain embodiments, the invention includes new compounds, and the administration of such compounds or their pharmaceutically acceptable salts, in an effective amount to a host, for example a human, to treat a coronavirus including SARS-COV2, either alone or in combination with another active antiviral or adjunctive agent.

Because of PLpro's unique mechanisms of action a compound of the present invention can both decrease viral replication directly and concurrently decrease the symptoms caused by the virus by inhibiting PLpro's protease activity in the host cell. For example, in certain embodiments a compound of the present invention or its pharmaceutically acceptable salt can inhibit the dysregulation of signaling cascades that are triggered by PLpro to lead to cell death of neighboring uninfected cells. By inhibiting the protease activity of PLpro, a compound of the present invention or its pharmaceutically acceptable salt can save the neighboring uninfected cells and thus decrease the viruses' symptoms in the host.

In one aspect, provided herein are compounds of formula (I′),

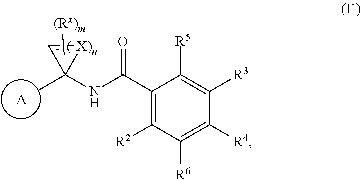

or a pharmaceutically acceptable salt thereof, wherein the variables are as defined herein.

In another aspect, provided herein are compounds of formula (I″),

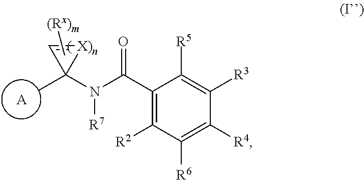

or a pharmaceutically acceptable salt thereof, wherein the variables are as defined herein.

In some embodiments, the compound of formula (I′) or (I″) is a compound of formula (Ia′):

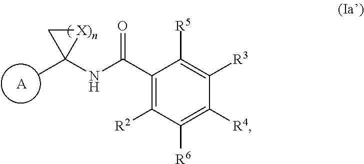

or a pharmaceutically acceptable salt thereof, wherein the variables are as defined herein.

In some embodiments, the compound of formula (I′) or (I″) is a compound of formula (Ib′):

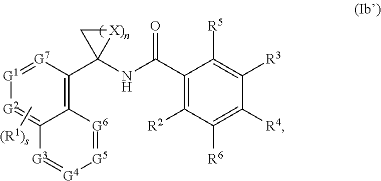

or a pharmaceutically acceptable salt thereof, wherein the variables are as defined herein.

In some embodiments, the compound of formula (I′) or (I″) is a compound of formula (Ic′):

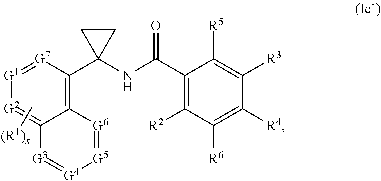

or a pharmaceutically acceptable salt thereof, wherein the variables are as defined herein.

In some embodiments, the compound of formula (I′) or (I″) is a compound of formula (Id′):

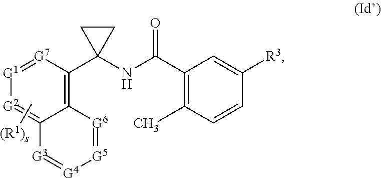

or a pharmaceutically acceptable salt thereof, wherein the variables are as defined herein.

In some embodiments, the compound of formula (I″) is a compound of formula (Ie′):

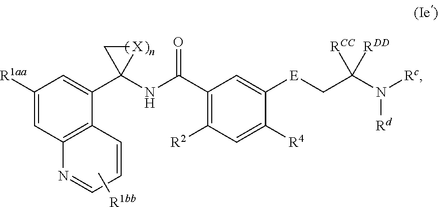

or a pharmaceutically acceptable salt thereof, wherein the variables are as defined herein.

In another aspect, provided herein are compounds of formula (I-1″),

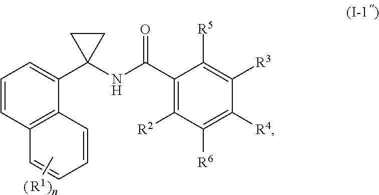

or a pharmaceutically acceptable salt thereof, wherein the variables are as defined herein.

In another aspect, provided herein are compounds of formula (II′),

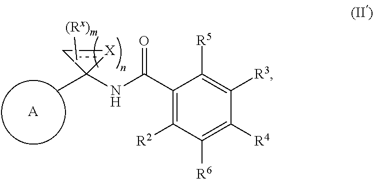

or a pharmaceutically acceptable salt thereof, wherein the variables are as defined herein.

In another aspect, provided herein are compounds of formula (II″),

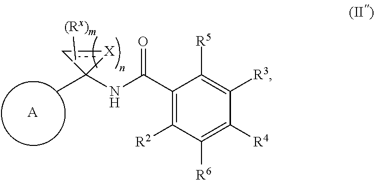

or a pharmaceutically acceptable salt thereof, wherein the variables are as defined herein.

In some embodiments, the compound of formula (II′) or (II″) is a compound of formula (IIa′):

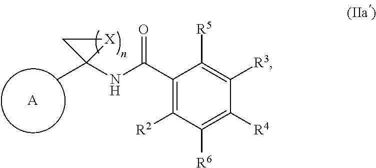

or a pharmaceutically acceptable salt thereof, wherein the variables are as defined herein.

In some embodiments, the compound of formula (II′) or (II″) is a compound of formula (IIb′):

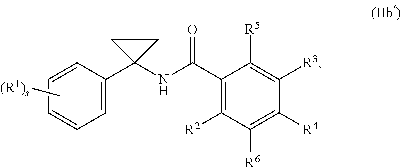

or a pharmaceutically acceptable salt thereof, wherein the variables are as defined herein.

In some embodiments, the compound of formula (II′) or (II″) is a compound of formula (IIc′):

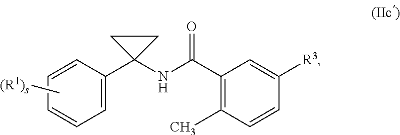

or a pharmaceutically acceptable salt thereof, wherein the variables are as defined herein.

In another aspect, provided herein are compounds of formula (III″),

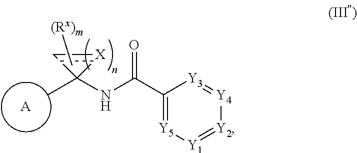

or a pharmaceutically acceptable salt thereof, wherein the variables are as defined herein.

In another aspect, provided herein are compounds of formula (IV″),

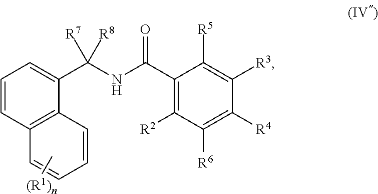

or a stereoisomer or a pharmaceutically acceptable salt thereof, wherein the variables are as defined herein.

In another aspect, provided here are compounds of formula (VI″):

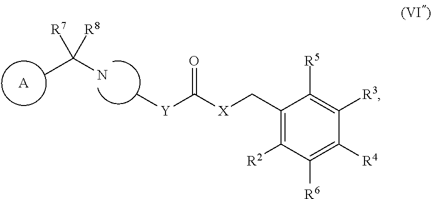

or a stereoisomer or a pharmaceutically acceptable salt thereof, wherein the variables are as defined herein.

In another aspect, provided herein are compounds of formula (VII″):

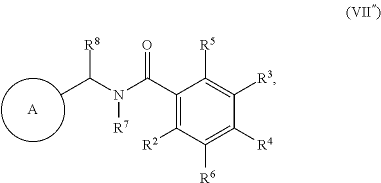

or a stereoisomer or a pharmaceutically acceptable salt thereof, wherein the variables are as defined herein.

In another aspect, provided herein are compounds of formula (VIII″)

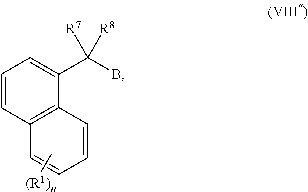

or a stereoisomer or a pharmaceutically acceptable salt thereof; wherein the variables are as defined herein.

In another aspect, provided herein are compounds of formula (IX″):

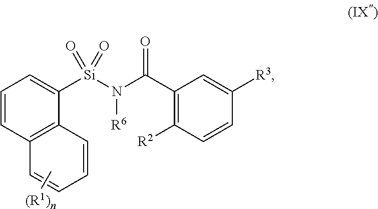

or a stereoisomer or a pharmaceutically acceptable salt thereof; wherein the variables are as defined herein.

In another aspect, provided herein are compounds of formula (X″)

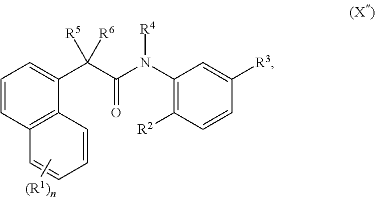

or a stereoisomer or a pharmaceutically acceptable salt thereof; wherein the variables are as defined herein.

In another aspect, provided herein is a pharmaceutical composition comprising a compound disclosed herein, or a pharmaceutically salt thereof, and one or more pharmaceutically acceptable excipients.

In another aspect, provided herein is a method of treating a viral infection in a subject in need thereof, the method comprising administering to the subject an effective amount of a compound or pharmaceutical composition disclosed herein.

In another aspect, provided herein is a method of preventing a viral infection in a subject in need thereof, the method comprising administering to the subject an effective amount of a compound or pharmaceutical composition disclosed herein

In some embodiments, the viral infection is a coronaviral infection. In some embodiments, the viral infection is caused by a coronavirus. In some embodiments, the coronavirus is SARS-COV-2.

In another aspect, provided herein is a method of inhibiting PLPro in a subject in need thereof, the method comprising administering to the subject an effective amount of a compound or pharmaceutical composition disclosed herein.

In another aspect, provided herein is a method of preventing replication of a virus in a subject in need thereof, the method comprising administering to the subject an effective amount of a compound or pharmaceutical composition disclosed herein.

In some embodiments, the virus is a coronavirus. In some embodiments, the coronavirus is SARS-Cov-2.

### DETAILED DESCRIPTION

This invention provides compounds for the inhibition of papain-like proteases (PLpros) which are essential for viral replication. In certain embodiments inhibition of PLpro directly inhibits viral replication. In certain embodiments inhibition of PLpro inhibits the dysregulation of signaling cascades in infected cells that absent inhibition leads to cell death in surrounding uninfected cells.

### Definitions

As used herein, the term “alkyl” refers to a saturated monovalent chain of carbon atoms, which may be optionally branched, the term “alkenyl” refers to an unsaturated monovalent chain of carbon atoms including at least one double bond, which may be optionally branched, the term “alkylene” refers to a saturated bivalent chain of carbon atoms, which may be optionally branched, and the term “cycloalkylene” refers to a saturated bivalent chain of carbon atoms, which may be optionally branched, a portion of which forms a ring.

As used herein, the term “alkynyl” refers to a monovalent chain of carbon atoms with one or more (e.g., one, two, three, or four) carbon-carbon triple bonds (e.g., 1, 2, 3, or 4 carbon-carbon triple bonds).

As used herein, the term “alkylene” is generally refers to a bivalent saturated hydrocarbon group wherein the hydrocarbon group may be a straight-chained or a branched-chain hydrocarbon group. Non-limiting illustrative examples include methylene, 1,2-ethylene, 1-methyl-1,2-ethylene, 1,4-butylene, 2,3-dimethyl-1,4-butylene, 2-methyl-2-ethyl-1,5-pentylene, and the like.

The term “cycloalkyl” as used herein generally refers to a monovalent, saturated hydrocarbon ring. The term “cycloalkenyl” as used herein refers to a monovalent hydrocarbon ring containing one or more (e.g., one, two, three, or four) unsaturated bonds.

As used herein, the term “carbocyclyl” or “carbocyclic” refers to a radical of a non-aromatic cyclic hydrocarbon group having from 3 to 12 ring carbon atoms (“3-12 membered carbocyclyl”) and zero heteroatoms in the non-aromatic ring system.

As used herein the term “isotopic derivative” refers to compounds wherein one or more (e.g., one, two, three, or four) atoms is isotopically enriched for example with more than about 50%, 60%, 70%, 80%, 90%, 95%, or 99% of an isotope. Isotopes are atoms with the same number of protons but a different number of neutrons (for example hydrogen has one proton and the isotope of hydrogen named deuterium has one proton and one neutron). Non-limiting examples of isotopes include isotopes of hydrogen (for example deuterium), carbon, nitrogen, oxygen, fluorine, and chlorine.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C6-14 aryl”). Aryl groups include, but are not limited to, phenyl and naphthyl. Unless otherwise specified, each instance of an aryl group is independently optionally substituted, i.e., unsubstituted (an “unsubstituted aryl”) or substituted (a “substituted aryl”) with one or more (e.g., one, two, three, or four) substituents.

The terms “bicycle” and “bicyclic” as used herein include molecular fragments or radicals that include two fused cycles wherein the cycles are independently selected from aryl, heteroaryl, cycloalkyl, heterocycle, and cycloalkenyl.

The terms “tricycle” and “tricyclic” as used herein include molecular fragments or radicals that include three fused cycles wherein the cycles are independently selected from aryl, heteroaryl, cycloalkyl, heterocycle, and cycloalkenyl.

The terms “heteroalkyl” and “heteroalkylene” as used herein includes molecular fragments or radicals comprising monovalent and divalent, respectively, groups that are formed from a linear or branched chain of carbon atoms and one or more (e.g., one, two, three, or four) heteroatoms, wherein the heteroatoms are selected from nitrogen, oxygen, and sulfur, such as alkoxyalkyl, alkyleneoxyalkyl, aminoalkyl, alkylaminoalkyl, alkyleneaminoalkyl, alkylthioalkyl, alkylenethioalkyl, alkoxyalkylaminoalkyl, alkylaminoalkoxyalkyl, alkyleneoxyalkylaminoalkyl, and the like. It is to be understood that neither heteroalkyl nor heteroalkylene includes oxygen-oxygen fragments. It is also to be understood that neither heteroalkyl nor heteroalkylene includes oxygen-sulfur fragments, unless the sulfur is oxidized as S(O) or S(O)₂.

As used herein, the terms “heterocycle” and “heterocyclyl” refer to a non-aromatic ring comprising one or more (e.g., one, two, three, or four) heteroatoms (e.g., N, O, and/or S), or monovalent radical thereof, respectively, wherein the heteroatoms are selected from nitrogen, oxygen, and sulfur, such as, but not limited to, tetrahydrofuran, aziridine, pyrrolidine, oxazolidine, 3-methoxypyrrolidine, 3-methylpiperazine, and the like. A heterocycle or heterocyclyl may be optionally fused to an aromatic or non-aromatic ring. As used herein, the term “heterocyclylene” refers to a bivalent radical of a heterocycle.

As used herein, the term “cycloheteroalkyl” generally refers to an optionally branched chain of atoms that includes both carbon and at least one heteroatom, where the chain optionally includes one or more unsaturated bonds, and where at least a portion of the chain forms one or more rings. As used herein, it is understood that the term “cycloheteroalkyl” also includes “heterocycloalkyl,” “heterocycle,” and “heterocyclyl.” The term “heterocycloalkenyl” as used herein refers to a monovalent chain of carbon atoms and heteroatoms containing one or more unsaturated bonds, a portion of which forms a ring, wherein the heteroatoms are selected from nitrogen, oxygen or sulfur. Illustrative cycloheteroalkyls include, but are not limited to, tetrahydrofuryl, bis(tetrahydrofuranyl), pyrrolidinyl, tetrahydropyranyl, piperidinyl, morpholinyl, piperazinyl, homopiperazinyl, quinuclidinyl, dihydrofuryl, pyrrollinyl, dihydropyranyl, and the like. It is also to be understood that cycloheteroalkyl includes polycyclic radicals, including fused bicycles, spiro bicycles, and the like.

As used herein, the term “acyl” refers to hydrogen, alkyl, cycloalkyl, alkenyl, cycloalkenyl, heterocyclyl, optionally substituted aryl, optionally substituted arylalkyl, optionally substituted heteroaryl, and optionally substituted heteroarylalkyl attached as a substituent through a carbonyl group, such as, but not limited to, formyl, acetyl, pivalolyl, benzoyl, phenacetyl, and the like.

“Heteroaryl” refers to a radical of a 5-14 membered monocyclic, bicyclic, or tricyclic 4n+2 aromatic ring system (e.g., having 6 or 10 pi electrons shared in a cyclic array) having ring carbon atoms and 1-4 ring heteroatoms (e.g., O, N, and/or S) provided in the aromatic ring system, wherein each heteroatom is independently selected from nitrogen, oxygen and sulfur (“5-12 membered heteroaryl”). In heteroaryl groups that contain one or more (e.g., one, two, three, or four) nitrogen atoms, the point of attachment can be a carbon or nitrogen atom, as valency permits. Heteroaryl bicyclic ring systems can include one or more (e.g., one, two, three, or four) heteroatoms in one or both rings. “Heteroaryl” also includes ring systems wherein the heteroaryl ring, as defined above, is fused with one or more (e.g., one, two, three, or four) aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). As used herein, the term “heteroarylene” refers to a divalent radical of a heteroaryl.

As used herein, “haloalkyl” is generally taken to mean an alkyl group wherein one or more (e.g., one, two, three, or four) hydrogen atoms is replaced with a halogen atom, independently selected in each instance from the group consisting of fluorine, chlorine, bromine and iodine. Non-limiting, illustrative examples include, difluoromethyl, 2,2,2-trifluoroethyl, 2-chlorobutyl, 2-chloro-2-propyl, trifluoromethyl, bromodifluoromethyl, and the like.

“Alkoxyl” or “alkoxy” refers to an alkyl group singularly bonded to an oxygen atom, having the formula R—O. Alkoxyls include, for example, methoxy (CH₃O—) and ethoxy, (CH₃CH₂O—). A “cycloalkoxyl” refers to a cycloalkyl group singularly bonded to an oxygen atom, which includes “aryloxy” groups, in which an aryl group is singular bonded to oxygen, for example a phenoxy group (C₆H₅O). Similarly, the term “heteroalkoxyl” refers to a heteroalkyl group singularly bonded to an oxygen atom and the term “cycloheteroalkoxyl” refers to a cycloheteroalkyl singularly bonded to an oxygen atom.

As used herein, the term “haloalkoxy” refers to a haloalkyl group which is attached to another moiety via an oxygen atom such as, e.g., —OCHCF₂ or —OCF₃.

“Halo” or “halogen,” independently or as part of another substituent, generally refers to a fluorine (F), chlorine (Cl), bromine (Br), or iodine (I) atom. The term “halide” by itself or as part of another substituent, refers to a fluoride, chloride, bromide, or iodide atom.

As used herein, the term “optionally substituted” includes a wide variety of groups that replace one or more (e.g., one, two, three, or four) hydrogens on a carbon, nitrogen, oxygen, or sulfur atom, including monovalent and divalent groups. For example, optional substitution of carbon includes, but is not limited to, halo, hydroxy, alkyl, alkoxy, haloalkyl, haloalkoxy, aryl, arylalkyl, acyl, acyloxy, and the like. In one aspect, optional substitution of aryl carbon includes, but is not limited to, halo, amino, hydroxy, alkyl, alkenyl, alkoxy, arylalkyl, arylalkyloxy, hydroxyalkyl, hydroxyalkenyl, alkylene dioxy, aminoalkyl, where the amino group may also be substituted with one or two alkyl groups, arylalkylgroups, and/or acylgroups, nitro, acyl and derivatives thereof such as oximes, hydrazones, and the like, cyano, alkylsulfonyl, alkylsulfonylamino, and the like. Illustratively, optional substitution of nitrogen, oxygen, and sulfur includes, but is not limited to, alkyl, haloalkyl, aryl, arylalkyl, acyl, and the like, as well as protecting groups, such as alkyl, ether, ester, and acyl protecting groups, and pro-drug groups. It is further understood that each of the foregoing optional substituents may themselves be additionally optionally substituted, such as with halo, hydroxy, alkyl, alkoxy, haloalkyl, haloalkoxy, and the like.

It is understood that substitutions and any functional group may be independently ortho-, para-, or meta-. It is understood that cyclic groups may be aromatic or non-aromatic.

“Stereoisomers”: It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R-and S-sequencing rules of Cahn and Prelog, or by the manner in which the molecule rotates the plane of polarized light and designated as dextrorotatory or levorotatory (i.e., as (+) or (−) isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci-and nitro-forms of phenylnitromethane, that are likewise formed by treatment with acid or base. Tautomeric forms may be relevant to the attainment of the optimal chemical reactivity and biological activity of a compound of interest.

### Compounds

In one aspect, provided herein are compounds of formula (I′),

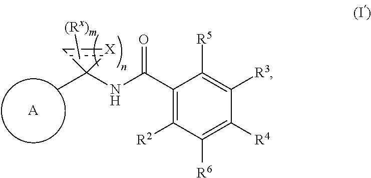

or a pharmaceutically acceptable salt thereof, wherein:
Ring A is selected from the group consisting of naphthyl, anthracenyl, 8-12 membered bicyclic or tricyclic heteroaryl, 8-12 membered bicyclic or tricyclic heterocyclyl, and 8-12 membered partially unsaturated bicyclic carbocyclyl, wherein the naphthyl, 8-12 membered bicyclic or tricyclic heteroaryl, and 8-12 membered partially unsaturated bicyclic carbocyclyl are optionally substituted by one or more (e.g., one, two, three, or four) R¹;
each X is independently selected from the group consisting of CH₂, CH, NR^{h}, and O (any hydrogen atom of the NH₂, CH, NRₕ (when Rₕ=H) may be substituted with Rₓ);
each R^{x} is independently C₁₋₆ alkyl or halo when R^{x} is substituted on a carbon atom or each R^{x} is independently C₁₋₆ alkyl when R^{x} is substituted on a nitrogen atom;
m is 0, 1, or 2;
[symbol] is a single bond or double bond;
n is 1 or 2;
each R¹ is independently selected from the group consisting of phenyl, 5-6 membered heteroaryl, C₃₋₇ carbocyclyl, 3-7 membered heterocyclyl, C₁₋₆ haloalkyl, C₁₋₆ haloalkoxy, halo, —CN, C₁₋₆ alkyl, C₂₋₆ alkenyl, C₂₋₆ alkynyl, C₁₋₆ alkoxy optionally substituted with phenyl, —O—C₁₋₆ alkylene-C₁₋₆ alkoxy, —C(O)ORⁱ, —S(O)ₜC₁₋₆alkyl, —S—C₁₋₆ haloalkyl, —NR^{e}R^{f}, —C(O)NR^{g}R^{h}, —O-phenyl, and hydroxy, wherein the phenyl and 5-6 membered heteroaryl are optionally substituted with one or more (e.g., one, two, three, or four) substituents each selected from the group consisting of halo, C₁₋₆ alkyl, C₁₋₆ haloalkyl, and C₁₋₆ alkoxy; wherein t is 0, 1, or 2;
R² is selected from the group consisting of halo, C₁₋₆ alkyl, C₁₋₆ haloalkyl, C₁₋₆ alkoxy, and C₃₋₆ cycloalkyl;
R³ is selected from the group consisting of C₁₋₆ alkoxy, hydroxy, C₀₋₆ alkylene-(3-8 membered heterocyclyl optionally substituted with one or more (e.g., one, two, three, or four) substituents each independently selected from the group consisting of C₁₋₆alkyl, (C₁₋₆alkylene-OH), and —NR^{e}R^{f}), —O—C₃₋₇ cycloalkyl optionally substituted with —NR^{c}R^{d}, —(C₀₋₆alkylene)-O-(3-8 membered heterocyclyl optionally substituted with one or more (e.g., one, two, three, or four) substituents each independently selected from the group consisting of C₁₋₆ alkyl, benzyl and —C(O)O—C₁₋₆ alkyl), C₁₋₆ alkyl optionally substituted with —NR^{c}R^{d}, —O-(5-6 membered heteroaryl), C₀₋₆ alkylene-CN, C₀₋₆ alkylene-C(O)O(C₁₋₆alkyl), C₀₋₆ alkylene-C(O)NR^{e}R^{f}, and —NR^{A}R^{B}, wherein the C₁₋₆ alkoxy is optionally substituted with one or more (e.g., one, two, three, or four) R^{3a};
R⁴ is selected from the group consisting of H, C₁₋₆ alkyl, halo, hydroxy, and —NR^{e}R^{f}, or
R³ and R⁴, together with the atoms to which they are attached, combine to form an aromatic or non-aromatic 5-10 membered monocyclic or bicyclic ring fused to the phenyl, wherein the 5-10 membered ring comprises at least one heteroatom, wherein the 5-10 membered ring is optionally substituted with one or more (e.g., one, two, three, or four) substituents each independently selected from the group consisting of C₁₋₆ alkyl optionally substituted with —NR^{c}R^{d}, C₃₋₇ cycloalkyl, phenyl, 5-6 membered heteroaryl, —(C₀₋₆ alkylene)-C(O)NR^{e}R^{f}, —C(O)—C₁₋₆heteroalkyl, —C(O)—NCH₃OCH₃, —(C₀₋₆ alkylene)-NR^{c}R^{d}, —(C₀₋₆ alkylene)-C₁₋₆ alkoxy, —(C₀₋₆ alkylene)-OH, oxo, —C(O)OH, and —(C₀₋₆ alkylene)-3-7 membered heterocyclyl optionally substituted with C₁₋₆alkyl;
R⁵ is selected from the group consisting of H, halo, and C₁₋₆ alkyl;
R⁶ is selected from the group consisting of H, halo, and C₁₋₆ alkyl;
each R^{3a} is independently selected from the group consisting of C₁₋₆ alkyl, —NR^{A}R^{B}, C₁₋₆ alkoxy, hydroxy, —C(O)NR^{c}R^{d}, 3-8 membered heterocyclyl, phenyl, 5-6 membered heteroaryl, and —(C₀₋₆ alkylene)-(C₃₋₆ cycloalkyl optionally substituted with NR^{c}R^{d}), wherein the 3-8 membered heterocyclyl and 5-6 membered heteroaryl are optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, phenyl, benzyl, C₁₋₆ alkylene-OH, and C₀₋₆ alkylene-C₁₋₆ alkoxy;
each R^{A} and R^{B} are independently, for each occurrence, selected from the group consisting of H, C₁₋₆ alkyl, C₁₋₆ haloalkyl, C₁₋₆ alkoxy, S(O)₂C₁₋₆ alkyl, —C(O)—C₁₋₆ alkyl, C₁₋₆ alkylene-NR^{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy or C₁₋₆ alkoxy;
each R^{c} and R^{d} are independently, for each occurrence, selected from the group consisting of H, C₁₋₆ alkyl, benzyl, and —C(O)OC₁₋₆ alkyl, or
R^{c} and R^{d} can be taken together with the nitrogen atom to which they are attached to form a 3-7 membered heterocyclyl;
each R^{e} and R^{f} are independently, for each occurrence, selected from the group consisting of H, C₁₋₆ alkyl, —C(O)C₁₋₆alkyl, and —C(O)OC₁₋₆alkyl; and
each R^{g}, R^{h}, and Rⁱ are independently, for each occurrence, H or C₁₋₆ alkyl.

In another aspect, provided herein are compounds of formula (I″),

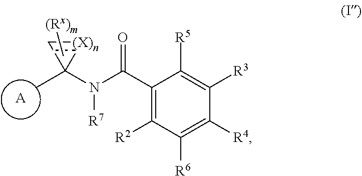

or a pharmaceutically acceptable salt thereof, wherein:
Ring A is selected from the group consisting of naphthyl, anthracenyl, phenanthrenyl, 8-14 membered bicyclic or tricyclic heteroaryl, 8-12 membered bicyclic or tricyclic heterocyclyl, and 8-12 membered partially unsaturated bicyclic carbocyclyl, wherein the naphthyl, 8-14 membered bicyclic or tricyclic heteroaryl, and 8-12 membered partially unsaturated bicyclic carbocyclyl are optionally substituted by one or more (e.g., one, two, three, or four) R¹,
wherein when ring A is 8-12 membered bicyclic heterocyclyl or 8-12 membered partially unsaturated bicyclic carbocyclyl, R³ is C₁₋₆ alkoxy optionally substituted with one or more (e.g., one, two, three, or four) R^{3a},
each X is independently selected from the group consisting of CH₂, CH, NR^{h}, and O (any hydrogen atom of the NH₂, CH, NRₕ (when Rₕ=H) may be substituted with Rₓ);
each R^{x} is independently D, C₁₋₆ alkyl or halo when R^{x} is substituted on a carbon atom or each R^{x} is independently C₁₋₆ alkyl when R^{x} is substituted on a nitrogen atom;
m is 0, 1, 2, 3, or 4;
[symbol] is a single bond or double bond;
n is 1 or 2;
each R¹ is independently selected from the group consisting of phenyl, 5-9 membered heteroaryl, C₃₋₇ carbocyclyl, 3-7 membered heterocyclyl optionally substituted with one or more (e.g., one, two, three, or four) R^{1a}, C₁₋₆ haloalkyl, C₁₋₆ haloalkoxy, halo, —CN, C₁₋₆ alkyl optionally substituted with hydroxy, C₂₋₆ alkenyl optionally substituted with C₃₋₆ cycloalkyl or phenyl substituted with C₀₋₆alkyl, C₂₋₆ alkynyl optionally substituted with one or more (e.g., one, two, three, or four) halo, C₁₋₆ alkoxy optionally substituted with phenyl, —O—C₁₋₆ alkylene-C₁₋₆ alkoxy, —C(O)—C₁₋₆ alkyl, —C(O)ORⁱ, —S(O)ₜC₁₋₆alkyl, —S—C₁₋₆ haloalkyl, —OS(O)ₜC₁₋₆haloalkyl, —NR^{j}R^{k}, —C(O)NR^{g}R^{h}, —O-phenyl, —B(OR^{m})₂, and hydroxy, wherein the phenyl and 5-9 membered heteroaryl are optionally substituted with one or more (e.g., one, two, three, or four) substituents each independently selected from the group consisting of halo, cyano, phenyl, 3-7 membered heterocyclyl, C₁₋₆ alkyl, C₁₋₆alkylene-(3-7 membered heterocyclyl optionally substituted with one or more (e.g., one, two, three, or four) halo), C₁₋₆alkylene substituted with hydroxy, C₁₋₆ alkylene-NR^{A}R^{B}, —C(O)-3-6 membered heterocyclyl, hydroxy, C₁₋₆ haloalkyl, C₁₋₆ alkoxy, and —C(O)—C₁₋₆ alkyl; wherein t is 0, 1, or 2;
R^{1a} is selected from the group consisting of oxo, halo, C₁₋₆haloalkyl, and C₁₋₆alkoxy;
R⁵ is selected from the group consisting of H, halo, and C₁₋₆ alkyl;
R⁶ is selected from the group consisting of H, halo, and C₁₋₆ alkyl;
R⁷ is selected from H and C₁₋₆ alkyl;
R² is selected from the group consisting of halo, C₁₋₆ alkyl, C₂₋₆ alkynyl, C₁₋₆ haloalkyl, C₁₋₆ alkoxy, and C₃₋₆ cycloalkyl;
R³ is selected from the group consisting of C₁₋₂₀ alkoxy, hydroxy, C₀₋₆ alkylene-(3-10 membered heterocyclyl optionally substituted with one or more (e.g., one, two, three, or four) substituents each independently selected from the group consisting of C₁₋₆alkyl, (C₁₋₆alkylene-OH), —C(O)O—C₁₋₆ alkyl, and —NR^{e}R^{f}, —O—C₃₋₇ cycloalkyl optionally substituted with —NR^{c}R^{d}, —(C₀₋₆alkylene)-O-(3-9 membered heterocyclyl optionally substituted with one or more (e.g., one, two, three, or four) substituents each independently selected from the group consisting of C₁₋₆ alkyl, benzyl and —C(O)O—C₁₋₆ alkyl), C₁₋₆ alkyl optionally substituted with —NR^{c}R^{d}, —O-(5-6 membered heteroaryl), C₀₋₆ alkylene-CN, C₀₋₆ alkylene-C(O)O(C₁₋₆alkyl), C₀₋₆ alkylene-C(O)NR^{e}R^{f}, and —NR^{AA}R^{BB}, wherein the C₁₋₂₀ alkoxy is optionally substituted with one or more (e.g., one, two, three, or four) R^{3a};
R⁴ is selected from the group consisting of H, C₁₋₆ alkyl, C₂₋₆ alkynyl optionally substituted with phenyl or 5-6 membered heteroaryl, halo, hydroxy, and —NR^{e}R^{f}, or
R³ and R⁴, together with the atoms to which they are attached, combine to form an aromatic or non-aromatic 5-10 membered monocyclic or bicyclic ring fused to the phenyl, wherein the 5-10 membered ring comprises at least one heteroatom, wherein the 5-10 membered ring is optionally substituted with one or more (e.g., one, two, three, or four) substituents each independently selected from the group consisting of C₁₋₆ alkyl optionally substituted with —NR^{c}R^{d}, C₃₋₇ cycloalkyl, phenyl, 5-6 membered heteroaryl, —(C₀₋₆ alkylene)-C(O)NR^{e}R^{f}, —C(O)—C₁₋₆heteroalkyl, —C(O)—NCH₃OCH₃, —(C₀₋₆ alkylene)-NR^{c}R^{d}, —(C₀₋₆ alkylene)-C₁₋₆ alkoxy, —(C₀₋₆ alkylene)-OH, oxo, —C(O)OH, and —(C₀₋₆ alkylene)-3-7 membered heterocyclyl optionally substituted with C₁₋₆alkyl, wherein the aromatic or non-aromatic 5-10 membered monocyclic or bicyclic ring fused to the phenyl is not an imidazole;
R^{AA} and R^{BB} are independently selected from the group consisting of H, C₁₋₆ alkyl, C₁₋₆ haloalkyl, C₁₋₆ alkoxy, S(O)₂C₁₋₆ alkyl, —C(O)—C₁₋₆ alkyl, C₁₋₂₀ alkylene-NR^{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, —(C₀₋₂₀ alkylene)-(3-10 membered heterocyclyl), and —(C₀₋₆ alkylene)-C₃₋₆ cycloalkyl, wherein the C₁₋₆ alkyl, C₁₋₆ alkylene, 3-10 membered heterocyclyl, and C₃₋₆ cycloalkyl are each optionally substituted with one or more (e.g., one, two, three, or four) substituents each independently selected from the group consisting of C₁₋₆ alkyl, hydroxy and C₁₋₆ alkoxy;
each R^{A} and R^{B} are independently, for each occurrence, selected from the group consisting of H, C₁₋₆ alkyl, C₁₋₆ haloalkyl, C₁₋₆ alkoxy, S(O)₂C₁₋₆ alkyl, —C(O)—C₁₋₆ alkyl, C₁₋₆ alkylene-NR^{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy or C₁₋₆ alkoxy;
each R^{c} and R^{d} are independently, for each occurrence, selected from the group consisting of H, C₁₋₆ alkyl, benzyl, and —C(O)OC₁₋₆ alkyl, or
R^{c} and R^{d} can be taken together with the nitrogen atom to which they are attached to form a 3-7 membered heterocyclyl;
each R^{e} and R^{f} are independently, for each occurrence, selected from the group consisting of H, C₁₋₆ alkyl, —C(O)C₁₋₆alkyl, —C(O)OC₁₋₆alkyl, and —S(O)₂C₁₋₆alkyl;
each R^{g}, R^{h}, and Rⁱ are independently, for each occurrence, H or C₁₋₆ alkyl; and
each R^{j} and R^{k} are independently, for each occurrence, selected from the group consisting of H, C₁₋₆ alkyl optionally substituted with phenyl, C₁₋₆ haloalkyl, —C(O)C₁₋₆alkyl, —S(O)₂C₁₋₆alkyl, and —C(O)OC₁₋₆alkyl;
each R^{3a} is independently selected from the group consisting of D, C₁₋₆ alkyl, —NR^{A}R^{B}, C₁₋₆ alkoxy, hydroxy, —C(O)NR^{c}R^{d}, 3-10 membered heterocyclyl, phenyl, 5-6 membered heteroaryl, and —(C₀₋₆ alkylene)-(C₃₋₆ cycloalkyl optionally substituted with NR^{c}R^{d}), wherein the 3-10 membered heterocyclyl and 5-6 membered heteroaryl are optionally substituted with one or more (e.g., one, two, three, or four) substituents each independently selected from the group consisting of halo, hydroxy, C₁₋₆ alkyl optionally substituted with one or more (e.g., one, two, three, or four) D, C₁₋₆ haloalkyl, —C(O)OC₁₋₆ alkyl, C₁₋₆ alkyelene-C₃₋₇ cycloalkyl, phenyl, benzyl, C₁₋₆ alkylene-OH, and C₀₋₆ alkylene-C₁₋₆ alkoxy; and
each R^{m} is independently selected from hydrogen and C₁₋₆alkyl or two (OR^{m}) groups can be taken together with the boron atom to which they are attached to form pinacol ester,

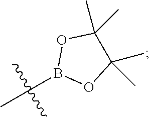 [SMILES: *B1OC(C)(C)C(C)(C)O1]

with the proviso that, when ring A is naphthyl, R⁴ is selected from the group consisting of hydrogen, —F, —Cl, C₁₋₆ alkyl, and —NR^{e}R^{f}; R⁵ is hydrogen; R⁶ is hydrogen or halo; X is CH₂; and n is 1;
   R² is selected from the group consisting of halo, C₁₋₆ alkyl, C₂₋₆ alkynyl, C₁₋₆ alkoxy, and C₃₋₆ cycloalkyl;
   R³ is selected from the group consisting of C₁₋₆ alkoxy, hydroxy, C₁₋₆ alkylene-(3-10 membered heterocyclyl optionally substituted with one or more (e.g., one, two, three, or four) R^{XX}), 3 membered heterocyclyl optionally substituted with one or more (e.g., one, two, three, or four) R^{XX}, 4 membered heterocyclyl substituted with one or more (e.g., one, two, three, or four) R^{XX}, 5 membered partially unsaturated heterocyclyl, 5 membered saturated heterocyclyl that has the point of attachment (to the phenyl) on the carbon atom of the heterocyclyl, 5 membered saturated heterocyclyl substituted with at least 2 (e.g., 2, 3, or 4) of R^{XX}, 6-membered heterocyclyl with at least 2 (e.g., 2, 3, or 4) of R^{XX} substituted on the carbon atoms of the heterocyclyl, —O—C₃₋₇ cycloalkyl optionally substituted with —NR^{c}R^{d}, —(C₀₋₆alkylene)-O-(3-9 membered heterocyclyl optionally substituted with one or more (e.g., one, two, three, or four) substituents each independently selected from the group consisting of C₁₋₆ alkyl, benzyl and —C(O)O—C₁₋₆ alkyl), C₁₋₆ alkyl optionally substituted with —NR^{c}R^{d}, —O-5-6 membered heteroaryl, C₀₋₆alkylene-CN, C₀₋₆ alkylene-C(O)O(C₁₋₆alkyl), C₀₋₆ alkylene-C(O)NR^{e}R^{f}, and —NR^{AA}R^{BB}, wherein the C₁₋₆ alkoxy is optionally substituted with one or more (e.g., one, two, three, or four) R^{3a}.
   each R^{XX} is independently selected from the group consisting of C₁₋₆alkyl, (C₁₋₆alkylene)-OH, —C(O)O—C₁₋₆ alkyl, and —NR^{e}R^{f}; and
R^{AA} and R^{BB} are independently selected from the group consisting of H, C₁₋₆ alkyl, C₁₋₆ haloalkyl, C₁₋₆ alkoxy, —C(O)—C₁₋₆ alkyl, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy or C₁₋₆ alkoxy, and at least one of R^{AA} and R^{BB} is not hydrogen; or R^{AA} is hydrogen and R^{BB} is C₁₋₆ alkylene-NR^{c}R^{d}.

In another aspect, provided herein are compounds of formula (I″-A),

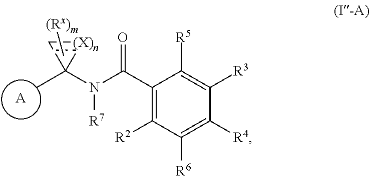

or a pharmaceutically acceptable salt thereof, wherein:
Ring A is selected from the group consisting of naphthyl, anthracenyl, phenanthrenyl, 8-14 membered bicyclic or tricyclic heteroaryl, 8-12 membered bicyclic or tricyclic heterocyclyl, and 8-12 membered partially unsaturated bicyclic carbocyclyl, wherein the naphthyl, 8-14 membered bicyclic or tricyclic heteroaryl, and 8-12 membered partially unsaturated bicyclic carbocyclyl are optionally substituted by one or more (e.g., one, two, three, or four) R¹,
wherein when ring A is 8-12 membered bicyclic heterocyclyl or 8-12 membered partially unsaturated bicyclic carbocyclyl, R³ is C₁₋₆ alkoxy optionally substituted with one or more (e.g., one, two, three, or four) R^{3a},
each X is independently selected from the group consisting of CH₂, CH, NR^{h}, and O (any hydrogen atom of the NH₂, CH, NRₕ (when Rₕ=H) may be substituted with Rₓ);
each R^{x} is independently D, C₁₋₆ alkyl or halo when R^{x} is substituted on a carbon atom or each R^{x} is independently C₁₋₆ alkyl when R^{x} is substituted on a nitrogen atom;
m is 0, 1, 2, 3, or 4;
[symbol] is a single bond or double bond;
n is 1 or 2;
each R¹ is independently selected from the group consisting of phenyl, 5-9 membered heteroaryl, C₃₋₇ carbocyclyl, 3-7 membered heterocyclyl optionally substituted with one or more (e.g., one, two, three, or four) R^{1a}, C₁₋₆ haloalkyl, C₁₋₆ haloalkoxy, halo, —CN, C₁₋₆ alkyl optionally substituted with hydroxy, C₂₋₆ alkenyl optionally substituted with C₃₋₆ cycloalkyl or phenyl substituted with C₀₋₆alkyl, C₂₋₆ alkynyl optionally substituted with one or more (e.g., one, two, three, or four) halo, C₁₋₆ alkoxy optionally substituted with phenyl, —O—C₁₋₆ alkylene-C₁₋₆ alkoxy, —C(O)—C₁₋₆ alkyl, —C(O)ORⁱ, —S(O)ₜC₁₋₆alkyl, —S—C₁₋₆ haloalkyl, —OS(O)ₜC₁₋₆haloalkyl, —NR^{j}R^{k}, —C(O)NR^{g}R^{h}, —O-phenyl, —B(OR^{m})₂, and hydroxy, wherein the phenyl and 5-9 membered heteroaryl are optionally substituted with one or more (e.g., one, two, three, or four) substituents each independently selected from the group consisting of halo, cyano, phenyl, 3-7 membered heterocyclyl, C₁₋₆ alkyl, C₁₋₆alkylene-(3-7 membered heterocyclyl optionally substituted with one or more (e.g., one, two, three, or four) halo), C₁₋₆alkylene substituted with hydroxy, C₁₋₆ alkylene-NR^{A}R^{B}, —C(O)-3-6 membered heterocyclyl, hydroxy, C₁₋₆ haloalkyl, C₁₋₆ alkoxy, and —C(O)—C₁₋₆ alkyl; wherein t is 0, 1, or 2;
R^{1a} is selected from the group consisting of oxo, halo, C₁₋₆haloalkyl, and C₁₋₆alkoxy;
R⁵ is selected from the group consisting of H, halo, and C₁₋₆ alkyl;
R⁶ is selected from the group consisting of H, halo, and C₁₋₆ alkyl;
R⁷ is selected from H and C₁₋₆ alkyl;
R² is selected from the group consisting of halo, C₁₋₆ alkyl, C₂₋₆ alkynyl, C₁₋₆ haloalkyl, C₁₋₆ alkoxy, and C₃₋₆ cycloalkyl;
R³ is selected from the group consisting of C₁₋₂₀ alkoxy, hydroxy, C₀₋₆ alkylene-(3-10 membered heterocyclyl optionally substituted with one or more (e.g., one, two, three, or four) substituents each independently selected from the group consisting of C₁₋₆alkyl, (C₁₋₆alkylene-OH), —C(O)O—C₁₋₆ alkyl, and —NR^{e}R^{f}, —O—C₃₋₇ cycloalkyl optionally substituted with —NR^{c}R^{d}, —(C₀₋₆alkylene)-O-(3-9 membered heterocyclyl optionally substituted with one or more (e.g., one, two, three, or four) substituents each independently selected from the group consisting of C₁₋₆ alkyl, benzyl and —C(O)O—C₁₋₆ alkyl), C₁₋₆ alkyl optionally substituted with —NR^{c}R^{d}, —O-(5-6 membered heteroaryl), C₀₋₆ alkylene-CN, C₀₋₆ alkylene-C(O)O(C₁₋₆alkyl), C₀₋₆ alkylene-C(O)NR^{e}R^{f}, and —NR^{AA}R^{BB}, wherein the C₁₋₂₀ alkoxy is optionally substituted with one or more (e.g., one, two, three, or four) R^{3a};
R⁴ is selected from the group consisting of H, C₁₋₆ alkyl, C₂₋₆ alkynyl optionally substituted with phenyl or 5-6 membered heteroaryl, halo, hydroxy, and —NR^{e}R^{f}, or
R³ and R⁴, together with the atoms to which they are attached, combine to form an aromatic or non-aromatic 5-10 membered monocyclic or bicyclic ring fused to the phenyl, wherein the 5-10 membered ring comprises at least one heteroatom, wherein the 5-10 membered ring is optionally substituted with one or more (e.g., one, two, three, or four) substituents each independently selected from the group consisting of C₁₋₆ alkyl optionally substituted with —NR^{c}R^{d}, C₃₋₇ cycloalkyl, phenyl, 5-6 membered heteroaryl, —(C₀₋₆ alkylene)-C(O)NR^{e}R^{f}, —C(O)—C₁₋₆heteroalkyl, —C(O)—NCH₃OCH₃, —(C₀₋₆ alkylene)-NR^{c}R^{d}, —(C₀₋₆ alkylene)-C₁₋₆ alkoxy, —(C₀₋₆ alkylene)-OH, oxo, —C(O)OH, and —(C₀₋₆ alkylene)-3-7 membered heterocyclyl optionally substituted with C₁₋₆alkyl, wherein the aromatic or non-aromatic 5-10 membered monocyclic or bicyclic ring fused to the phenyl is not an imidazole;
R^{AA} and R^{BB} are independently selected from the group consisting of H, C₁₋₆ alkyl, C₁₋₆ haloalkyl, C₁₋₆ alkoxy, S(O)₂C₁₋₆ alkyl, —C(O)—C₁₋₆ alkyl, C₁₋₂₀ alkylene-NR^{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, —(C₀₋₂₀ alkylene)-(3-10 membered heterocyclyl), and —(C₀₋₆ alkylene)-C₃₋₆ cycloalkyl, wherein the C₁₋₆ alkyl, C₁₋₆ alkylene, 3-10 membered heterocyclyl, and C₃₋₆ cycloalkyl are each optionally substituted with one or more (e.g., one, two, three, or four) substituents each independently selected from the group consisting of C₁₋₆ alkyl, hydroxy and C₁₋₆ alkoxy;
each R^{A} and R^{B} are independently, for each occurrence, selected from the group consisting of H, C₁₋₆ alkyl, C₁₋₆ haloalkyl, C₁₋₆ alkoxy, S(O)₂C₁₋₆ alkyl, —C(O)—C₁₋₆ alkyl, C₁₋₆ alkylene-NR^{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy or C₁₋₆ alkoxy;
each R^{e} and R^{d} are independently, for each occurrence, selected from the group consisting of H, C₁₋₆ alkyl, benzyl, and —C(O)OC₁₋₆ alkyl, or
R^{c} and R^{d} can be taken together with the nitrogen atom to which they are attached to form a 3-7 membered heterocyclyl;
each R^{e} and R^{f} are independently, for each occurrence, selected from the group consisting of H, C₁₋₆ alkyl, —C(O)C₁₋₆alkyl, —C(O)OC₁₋₆alkyl, and —S(O)₂C₁₋₆alkyl;
each R^{g}, R^{h}, and Rⁱ are independently, for each occurrence, H or C₁₋₆ alkyl; and
each R^{j} and R^{k} are independently, for each occurrence, selected from the group consisting of H, C₁₋₆ alkyl optionally substituted with phenyl, C₁₋₆ haloalkyl, —C(O)C₁₋₆alkyl, —S(O)₂C₁₋₆alkyl, and —C(O)OC₁₋₆alkyl;
each R^{3a} is independently selected from the group consisting of D, C₁₋₆ alkyl, —NR^{A}R^{B}, C₁₋₆ alkoxy, hydroxy, —C(O)NR^{c}R^{d}, 3-10 membered heterocyclyl, phenyl, 5-6 membered heteroaryl, and —(C₀₋₆ alkylene)-(C₃₋₆ cycloalkyl optionally substituted with NR^{c}R^{d}), wherein the 3-10 membered heterocyclyl and 5-6 membered heteroaryl are optionally substituted with one or more (e.g., one, two, three, or four) substituents each independently selected from the group consisting of halo, hydroxy, C₁₋₆ alkyl optionally substituted with one or more (e.g., one, two, three, or four) D, C₁₋₆ haloalkyl, —C(O)OC₁₋₆ alkyl, C₁₋₆ alkyelene-C₃₋₇ cycloalkyl, phenyl, benzyl, C₁₋₆ alkylene-OH, and C₀₋₆ alkylene-C₁₋₆ alkoxy; and
each R^{m} is independently selected from hydrogen and C₁₋₆alkyl or two (OR^{m}) groups can be taken together with the boron atom to which they are attached to form pinacol ester,

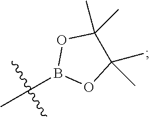 [SMILES: *B1OC(C)(C)C(C)(C)O1]

and
R^{AA} and R^{BB} are independently selected from the group consisting of H, C₁₋₆ alkyl, C₁₋₆ haloalkyl, C₁₋₆ alkoxy, —C(O)—C₁₋₆ alkyl, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy or C₁₋₆ alkoxy, and at least one of R^{AA} and R^{BB} is not hydrogen; or R^{AA} is hydrogen and R^{BB} is C₁₋₆ alkylene-NR^{c}R^{d}.

In some embodiments, the compound of formula (I′), (I″), or (I″-A) is a compound of formula (Ia′):

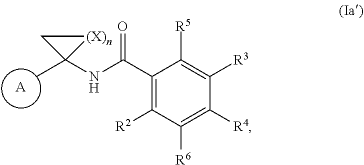

or a pharmaceutically acceptable salt thereof, wherein the variables are as defined above.

In some embodiments, the compound of formula (I′), (I″), or (I″-A) is a compound of formula (Ib′):

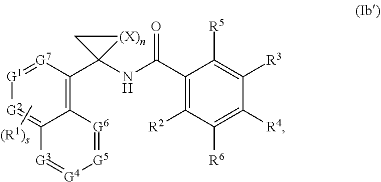

or a pharmaceutically acceptable salt thereof, wherein:
each of G¹, G², G³, G⁴, G⁵, G⁶, and G⁷ is independently selected from CH and N;
s is 0, 1, 2, or 3;

wherein X, n, R¹, R², R³, R⁴, R⁵, and R⁶ are as defined above.

In some embodiments, the compound of formula (I′), (I″), or (I″-A) is a compound of formula (Ic′):

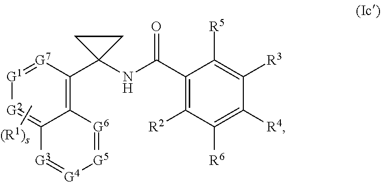

or a pharmaceutically acceptable salt thereof, wherein the variables are as defined above.

In some embodiments, the compound of formula (I′), (I″), or (I″-A) is a compound of formula (Id′):

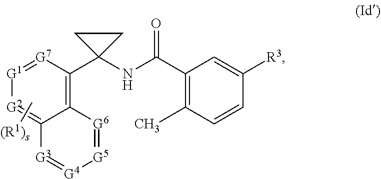

or a pharmaceutically acceptable salt thereof, wherein the variables are as defined above.

In some embodiments, compounds of the disclosure have a structure of formula (I′), (I″), or (I″-A), wherein G¹, G², G³, G⁴, G⁵, G⁶, and G⁷ are CH. In some embodiments, compounds of the disclosure have a structure of formula (I′), (I″), or (I″-A), wherein G¹ is N, and G², G³, G⁴, G⁵, G⁶, and G⁷ are CH; or G² is N, and G¹, G³, G⁴, G⁵, G⁶, and G⁷ are CH. In some embodiments, compounds of the disclosure have a structure of formula (I′), (I″), or (I″-A), wherein G³ is N, and G¹, G², G⁴, G⁵, G⁶, and G⁷ are CH; or G⁶ is N, and G¹, G², G³, G⁵, G⁶, and G⁷ are CH. In some embodiments, compounds of the disclosure have a structure of formula (I′), (I″), or (I″-A), wherein G⁵ is N, and G¹, G², G³, G⁴, G⁶, and G⁷ are CH; or G⁶ is N, and G¹, G², G³, G⁴, G⁵, and G⁷ are CH. In some embodiments, compounds of the disclosure have a structure of formula (I′), (I″), or (I″-A), wherein G⁷ is N, and G¹, G², G³, G⁴, G⁵, and G⁶ are CH.

In some embodiments, compounds of the disclosure have a structure of formula (I′), (I″), or (I″-A), wherein s is 0. In some embodiments, compounds of the disclosure have a structure of formula (I′), (I″), or (I″-A), wherein s is 1. In some embodiments, compounds of the disclosure have a structure of formula (I′), (I″), or (I″-A), wherein s is 2.

In some embodiments, compounds of the disclosure have a structure of formula (I′), (I″), or (I″-A), wherein Ring A is naphthyl.

In some embodiments, compounds of the disclosure have a structure of formula (I′), (I″), or (I″-A), wherein Ring A is selected from

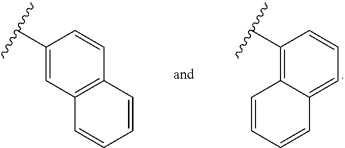 [SMILES: *c1ccc2ccccc2c1 ; *c1cccc2ccccc12]

In some embodiments, compounds of the disclosure have a structure of formula (I″) or (I″-A), wherein Ring A is anthracenyl. In some embodiments, compounds of the disclosure have a structure of formula (I″) or (I″-A), wherein Ring A is phenanthrenyl.

In some embodiments, compounds of the disclosure have a structure of formula (I″) or (I″-A), wherein Ring A is 8-14 membered bicyclic or tricyclic heteroaryl. In some embodiments, Ring A is 10-membered bicyclic heteroaryl. In some embodiments, compounds of the disclosure have a structure of formula (I′), (I″), or (I″-A), wherein Ring A is quinolinyl.

In some embodiments, compounds of the disclosure have a structure of formula (I′), (I″), or (I″-A), wherein Ring A is selected from the group consisting of

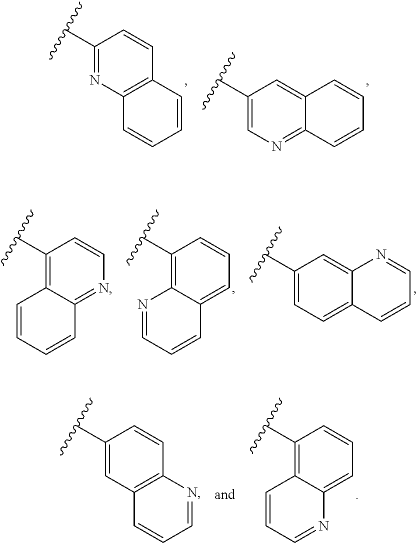

In some embodiments, compounds of the disclosure have a structure of formula (I′), (I″), or (I″-A), wherein Ring A is

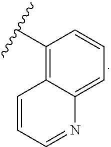 [SMILES: *c1cccc2ncccc12]

In some embodiments, compounds of the disclosure have a structure of formula (I′), (I″), or (I″-A), wherein Ring A is isoquinolinyl,

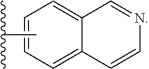

In some embodiments, compounds of the disclosure have a structure of formula (I′), (I″), or (I″-A), wherein Ring A is selected from the group consisting of

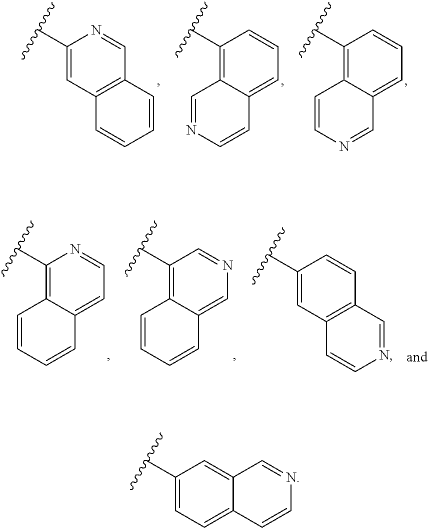

In some embodiments, the compound of formula (I″) or (I″-A) is a compound of formula (Ie′):

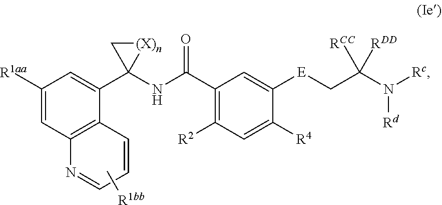

or a pharmaceutically acceptable salt thereof, wherein:

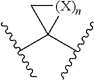

represents cyclopropylene or oxetan-3-ylene (i.e.,

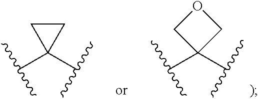 [SMILES: *C1(*)CC1 ; *C1(*)COC1]

R^{1aa} is selected from the group consisting of halo, C₁₋₆ alkyl, C₂₋₆ alkenyl, C₂₋₆ alkynyl, C₁₋₆ alkoxy, C₁₋₆ haloalkyl, —NH(C₁₋₆ alkyl), —N(C₁₋₆ alkyl)₂, 5- or 6-membered monocyclic heteroaryl, 4- or 5-membered heterocyclyl;
R^{1bb} is selected from the group consisting of H, halo, C₁₋₆ alkyl, C₁₋₆ alkoxy, and C₁₋₆ haloalkyl;
R² is selected from the group consisting of halo, C₁₋₆ alkyl, and C₁₋₆ haloalkyl;
R⁴ is selected from the group consisting of H, hydroxy, —NH₂, —NH(C₁₋₆ alkyl), —N(C₁₋₆ alkyl)₂, C₁₋₆ alkyl, C₁₋₆ alkoxy, and C₁₋₆ haloalkyl;
E is selected from the group consisting of —O—, —N(H)—, and —N(C₁₋₆ alkyl)-;
R^{CC} is H or C₁₋₂ alkyl;
R^{DD} is H or C₁₋₂ alkyl;
R^{c} is H or C₁₋₆ alkyl; or
R^{CC} is H, and R^{DD} and R^{c} can be taken together with the carbon and nitrogen atoms to which they are attached to form a 4- to 5-membered heterocycle; and
   R^{d} is H or C₁₋₆ alkyl.

In some embodiments, compounds of the disclosure have a structure of formula (Ie′), wherein

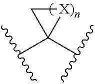

is cyclopropylene (i.e.,

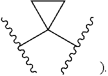 [SMILES: *C1(*)CC1]

In some embodiments, compounds of the disclosure have a structure of formula (Ie′), wherein

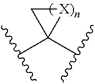

represents oxetan-3ylene (i.e.,

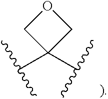 [SMILES: *C1(*)COC1]

In some embodiments, compounds of the disclosure have a structure of formula (Ie′), wherein R^{1aa} is C₁₋₆ alkoxy. In some embodiments, compounds of the disclosure have a structure of formula (Ie′), wherein R^{1aa} is C₂₋₆ alkenyl.

In some embodiments, compounds of the disclosure have a structure of formula (Ie′), wherein Rhu 1bb is H. In some embodiments, compounds of the disclosure have a structure of formula (Ie′), wherein Rhu 1bb is C₁₋₆ alkyl.

In some embodiments, compounds of the disclosure have a structure of formula (Ie′), wherein R² is C₁₋₆ alkyl.

In some embodiments, compounds of the disclosure have a structure of formula (Ie′), wherein E is —O—. In some embodiments, compounds of the disclosure have a structure of formula (Ie′), wherein E is —N(H)—. In some embodiments, compounds of the disclosure have a structure of formula (Ie′), wherein E is —N(C₁₋₆ alkyl)-.

In some embodiments, compounds of the disclosure have a structure of formula (Ie′), wherein R^{CC} is H, and R^{DD} and R^{e} are taken together with the carbon and nitrogen atoms to which they are attached to form a 4- to 5-membered heterocycle.

In some embodiments, compounds of the disclosure have a structure of formula (Ie′), wherein R^{d} is H. In some embodiments, compounds of the disclosure have a structure of formula (Ie′), wherein R^{d} is C₁₋₆ alkyl.

In some embodiments, the compound of formula (I′), (I″), or (I″-A) is a compound of formula (Ie″):

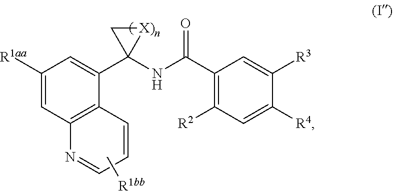

or a pharmaceutically acceptable salt thereof, wherein:

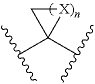

represents cyclopropylene or oxetan-3-ylene (i.e.,

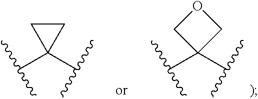 [SMILES: *C1(*)CC1 ; *C1(*)COC1]

R^{1aa} is selected from the group consisting of halo, C₁₋₆ alkyl, C₂₋₆ alkenyl, C₂₋₆ alkynyl, C₁₋₆ alkoxy, C₁₋₆ haloalkyl, —NH(C₁₋₆ alkyl), —N(C₁₋₆ alkyl)₂, 5- or 6-membered monocyclic heteroaryl, 4- or 5-membered heterocyclyl;
R^{1bb} is selected from the group consisting of H, halo, C₁₋₆ alkyl, C₁₋₆ alkoxy, and C₁₋₆ haloalkyl;
R² is selected from the group consisting of halo, C₁₋₆ alkyl, and C₁₋₆ haloalkyl;
R⁴ is selected from the group consisting of H, hydroxy, —NH₂, —NH(C₁₋₆ alkyl), —N(C₁₋₆ alkyl)₂, and C₁₋₆ alkyl;
R³ is selected from C₁₋₆ alkoxy or —NR^{A}R^{B}, wherein the C₁₋₆ alkoxy is optionally substituted with one or more (e.g., one, two, three, or four) 3-8 membered heterocyclyl, wherein the 3-8 membered heterocyclyl is optionally substituted with C₁₋₆ alkyl; and
R^{A} and R^{B} are independently selected from the group consisting of H, C₁₋₆ alkyl, and —C₁₋₆ alkylene-NR^{c}R^{d}, wherein R^{c} and R^{d} are independently selected from H and C₁₋₆ alkyl.

In some embodiments, compounds of the disclosure have a structure of formula (Ie″), wherein

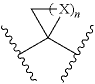

is cyclopropylene (i.e.,

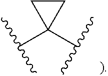 [SMILES: *C1(*)CC1]

In some embodiments, compounds of the disclosure have a structure of formula (Ie″), wherein

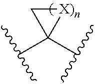

represents oxetan-3-ylene (i.e.,

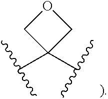 [SMILES: *C1(*)COC1]

In some embodiments, compounds of the disclosure have a structure of formula (Ie″), wherein R^{1aa} is C₁₋₆ alkoxy. In some embodiments, compounds of the disclosure have a structure of formula (Ie″), wherein R^{1aa} is C₂₋₆ alkenyl.

In some embodiments, compounds of the disclosure have a structure of formula (Ie″), wherein Rhu 1bb is H. In some embodiments, compounds of the disclosure have a structure of formula (Ie″), wherein Rhu 1bb is C₁₋₆ alkyl.

In some embodiments, compounds of the disclosure have a structure of formula (Ie″), wherein R² is C₁₋₆ alkyl.

In some embodiments, compounds of the disclosure have a structure of formula (Ie″), wherein R³ is C₁₋₆ alkoxy. In some embodiments, the C₁₋₆ alkoxy is substituted with 3-8 membered heterocyclyl. In some embodiments, the C₁₋₆ alkoxy is substituted with 3-8 membered heterocyclyl substituted with methyl.

In some embodiments, compounds of the disclosure have a structure of formula (Ie″), wherein R³ is NR^{A}R^{B}.

In some embodiments, compounds of the disclosure have a structure of formula (Ie″), wherein R^{A} is —C₁₋₆ alkylene-NR^{c}R^{d} and R^{B} is H or C₁₋₆ alkyl.

In some embodiments, compounds of the disclosure have a structure of formula (Ie″), wherein R^{c} and R^{d} are C₁₋₆ alkyl. In some embodiments, compounds of the disclosure have a structure of formula (Ie″), wherein R^{c} and R^{d} are H. In some embodiments, compounds of the disclosure have a structure of formula (Ie″), wherein R^{e} is C₁₋₆ alkyl and R^{d} is H.

In some embodiments, compounds of the disclosure have a structure of formula (Ie″), wherein R^{A} and R^{B} are C₁₋₆ alkyl. In some embodiments, compounds of the disclosure have a structure of formula (Ie″), wherein R^{A} and R^{B} are H. In some embodiments, compounds of the disclosure have a structure of formula (Ie″), wherein R^{A} is C₁₋₆ alkyl and R^{B} is H.

In some embodiments, compounds of the disclosure have a structure of formula (I′), (I″), or (I″-A), wherein X is CH₂. In some embodiments, compounds of the disclosure have a structure of formula (I′), (I″), or (I″-A), wherein X is CH. In some embodiments, compounds of the disclosure have a structure of formula (I′), (I″), or (I″-A), wherein X is NH. In some embodiments, compounds of the disclosure have a structure of f formula (I′), (I″), or (I″-A), wherein X is O.

In some embodiments, compounds of the disclosure have a structure of formula (I′), (I″), or (I″-A), wherein n is 2 and one of X is O and the other X is CH₂.

In some embodiments, compounds of the disclosure have a structure of formula (I′), (I″), or (I″-A), wherein m is 0. In some embodiments, compounds of the disclosure have a structure of formula (I′), (I″), or (I″-A), wherein m is 1. In some embodiments, compounds of the disclosure have a structure of formula (I′), (I″), or (I″-A), wherein m is 2. In some embodiments, compounds of the disclosure have a structure of formula (I″) or (I″-A), wherein m is 3. In some embodiments, compounds of the disclosure have a structure of formula (I″) or (I″-A), wherein m is 4.

In some embodiments, compounds of the disclosure have a structure of formula (I′), (I″), or (I″-A), wherein [symbol] is a single bond. In some embodiments, compounds of the disclosure have a structure of formula (I′), (I″), or (I″-A), wherein [symbol] is a double bond.

In some embodiments, compounds of the disclosure have a structure of formula (I′), (I″), or (I″-A), wherein n is 1. In some embodiments, compounds of the disclosure have a structure of formula (I′), (I″), or (I″-A), wherein n is 2.

In some embodiments, compounds of the disclosure have a structure of f formula (I′), (I″), or (I″-A), wherein each R¹ is independently selected from the group consisting of phenyl, 5-6 membered heteroaryl, C₃₋₇ carbocyclyl, C₁₋₆ haloalkyl, halo, —CN, C₁₋₆ alkyl, C₂₋₆ alkenyl, C₁₋₆ alkoxy, —S(O)—C₁₋₆alkyl, —NR^{e}R^{f}, and hydroxy.

In some embodiments, compounds of the disclosure have a structure of formula (I″) or (I″-A), wherein each R¹ is independently selected from the group consisting of phenyl, 5-9 membered heteroaryl, C₃₋₇ carbocyclyl, 3-7 membered heterocyclyl optionally substituted with one or more (e.g., one, two, three, or four) R^{1a}, C₁₋₆ haloalkyl, halo, —CN, C₁₋₆ alkyl optionally substituted with hydroxy, C₂₋₆ alkenyl optionally substituted with C₃₋₆ cycloalkyl or phenyl substituted with C₀₋₆alkyl, C₂₋₆ alkynyl; optionally substituted with one or more (e.g., one, two, three, or four) halo, C₁₋₆ alkoxy optionally substituted with phenyl, —S(O), —C₁₋₆alkyl, —C(O)—C₁₋₆ alkyl, OS(O)ₜC₁₋₆haloalkyl, —NR^{j}R^{k} and hydroxy.

In some embodiments, compounds of the disclosure have a structure of formula (I′), (I″), or (I″-A), wherein each R¹ is independently selected from the group consisting of phenyl, 5-6 membered heteroaryl, C₃₋₇ carbocyclyl, C₁₋₆ haloalkyl, halo, and C₁₋₆ alkyl, wherein the phenyl and 5-6 membered heteroaryl are optionally substituted with one or more (e.g., one, two, three, or four) substituents each independently selected from the group consisting of halo, C₁₋₆ alkyl, C₁₋₆ haloalkyl, and C₁₋₆ alkoxy.

In some embodiments, compounds of the disclosure have a structure of formula (I″) or (I″-A), wherein each R¹ is independently selected from the group consisting of phenyl, 5-9 membered heteroaryl, C₃₋₇ carbocyclyl, C₁₋₆ haloalkyl, halo, and C₁₋₆ alkyl, wherein the phenyl and 5-9 membered heteroaryl are optionally substituted with one or more (e.g., one, two, three, or four) substituents each independently selected from the group consisting of halo, C₁₋₆ alkyl, C₁₋₆ haloalkyl, and C₁₋₆ alkoxy.

In some embodiments, compounds of the disclosure have a structure of formula (I″), or (I″-A), wherein each R¹ is independently selected from the group consisting of 5-9 membered heteroaryl, 3-7 membered heterocyclyl optionally substituted with one or more (e.g., one, two, three, or four) R^{1a}, C₁₋₆ haloalkyl, halo, C₁₋₆ alkyl optionally substituted with hydroxy, C₂₋₆ alkenyl optionally substituted with C₃₋₆ cycloalkyl or phenyl substituted with C₀₋₆alkyl, C₂₋₆ alkynyl optionally substituted with one or more (e.g., one, two, three, or four) halo, C₁₋₆ alkoxy optionally substituted with phenyl, —NR^{j}R^{k}, wherein the 5-9 membered heteroaryl is optionally substituted with one or more (e.g., one, two, three, or four) substituents each selected from the group consisting of halo, cyano, phenyl, 3-7 membered heterocyclyl, C₁₋₆ alkyl, C₁₋₆alkylene-(3-7 membered heterocyclyl optionally substituted with one or more (e.g., one, two, three, or four) halo), C₁₋₆alkylene substituted with hydroxy, C₁₋₆ alkylene-NR^{A}R^{B}, —C(O)-3-6 membered heterocyclyl, hydroxy, C₁₋₆ haloalkyl, C₁₋₆ alkoxy, and —C(O)—C₁₋₆ alkyl.

In some embodiments, compounds of the disclosure have a structure of formula (I″), or (I″-A), wherein each R¹ is independently selected from the group consisting of 5-9 membered heteroaryl, 3-7 membered heterocyclyl, C₁₋₆ haloalkyl, halo, C₁₋₆ alkyl, C₂₋₆ alkenyl, C₂₋₆ alkynyl, C₁₋₆ alkoxy, and —NR^{j}R^{k}.

In some embodiments, compounds of the disclosure have a structure of formula (I″), or (I″-A), wherein each R¹ is selected from the group consisting of halo, C₁₋₆ alkyl, C₂₋₆ alkenyl, C₂₋₆ alkynyl, C₁₋₆ alkoxy, C₁₋₆ haloalkyl, —NH(C₁₋₆ alkyl), —N(C₁₋₆ alkyl)₂, 5- or 6-membered monocyclic heteroaryl, and 4- or 5-membered heterocyclyl.

In some embodiments, compounds of the disclosure have a structure of formula (I″), or (I″-A), wherein each R¹ is selected from the group consisting of H, halo, C₁₋₆ alkyl, C₁₋₆ alkoxy, and C₁₋₆ haloalkyl.

In some embodiments, compounds of the disclosure have a structure of formula (I′), (I″), or (I″-A), wherein R₂ is selected from the group consisting of halo, C₁₋₆ alkyl, C₁₋₆ alkoxy, and C₃₋₆ cycloalkyl. In some embodiments, compounds of the disclosure have a structure of formula (I′), (I″), or (I″-A), wherein R₂ is selected from the group consisting of halo, C₁₋₆ alkyl, C₁₋₆ alkoxy, C₂₋₆ alkynyl, and C₃₋₆ cycloalkyl. In some embodiments, compounds of the disclosure have a structure of formula (I′), (I″), or (I″-A), wherein R² is selected from the group consisting of halo, C₁₋₆ alkyl, and C₁₋₆ haloalkyl. In some embodiments, compounds of the disclosure have a structure of formula (I′), (I″), or (I″-A), wherein R² is halo. In some embodiments, compounds of the disclosure have a structure of formula (I′), (I″), or (I″-A), wherein R² is C₁₋₆ alkyl.

In some embodiments, compounds of the disclosure have a structure of formula (I′), wherein R³ is selected from the group consisting of C₁₋₆ alkoxy, hydroxy, C₀₋₆ alkylene-(3-8 membered heterocyclyl optionally substituted with one or more (e.g., one, two, three, or four) substituents each independently selected from the group consisting of C₁₋₆alkyl, (C₁₋₆alkylene-OH), and —NR^{e}R^{f}), —O—C₃₋₇ cycloalkyl optionally substituted with —NR^{c}R^{d}, —(C₀₋₆alkylene)-(O-3-8 membered heterocyclyl optionally substituted with one or more (e.g., one, two, three, or four) substituents each independently selected from the group consisting of C₁₋₆ alkyl benzyl, and —C(O)O—C₁₋₆ alkyl), and —NR^{A}R^{B}, wherein the C₁₋₆ alkoxy is optionally substituted with one or more (e.g., one, two, three, or four) R^{3a}.

In some embodiments, compounds of the disclosure have a structure of formula (I″) or (I″-A), wherein R³ is selected from the group consisting of C₁₋₆ alkoxy, hydroxy, C₀₋₆ alkylene-(3-10 membered heterocyclyl optionally substituted with one or more (e.g., one, two, three, or four) substituents each independently selected from the group consisting of C₁₋₆alkyl, (C₁₋₆alkylene-OH), —C(O)O—C₁₋₆ alkyl, and —NR^{e}R^{f}, —O—C₃₋₇ cycloalkyl optionally substituted with —NR^{c}R^{d}, —(C₀₋₆alkylene)-(O-3-9 membered heterocyclyl optionally substituted with one or more (e.g., one, two, three, or four) substituents each independently selected from the group consisting of C₁₋₆ alkyl benzyl, and —C(O)O—C₁₋₆ alkyl), and NR^{AA}R^{BB}, wherein the C₁₋₆ alkoxy is optionally substituted with one or more (e.g., one, two, three, or four) R^{3a}.

In some embodiments, compounds of the disclosure have a structure of formula (I′), (I″), or (I″-A), wherein R³ is selected from the group consisting of C₁₋₆ alkoxy and —(C₀₋₆alkylene)-O-(3-8 membered heterocyclyl optionally substituted with one or more (e.g., one, two, three, or four) substituents each independently selected from the group consisting of C₁₋₆ alkyl, benzyl and —C(O)O—C₁₋₆ alkyl), —O—C₃₋₇ cycloalkyl optionally substituted with —NR^{c}R^{d}, wherein the C₁₋₆ alkoxy is optionally substituted with one or more (e.g., one, two, three, or four) R^{3a}.

In some embodiments, compounds of the disclosure have a structure of formula (I′), (I″), or (I″-A), wherein R³ is selected from the group consisting of C₁₋₆ alkoxy and —(C₀₋₆alkylene)-O-(3-9 membered heterocyclyl optionally substituted with one or more (e.g., one, two, three, or four) substituents each independently selected from the group consisting of C₁₋₆ alkyl, benzyl and —C(O)O—C₁₋₆ alkyl), —O—C₃₋₇ cycloalkyl optionally substituted with —NR^{c}R^{d}, wherein the C₁₋₆ alkoxy is optionally substituted with one or more (e.g., one, two, three, or four) R^{3a}.

In some embodiments, compounds of the disclosure have a structure of formula (I″) or (I″-A), wherein R³ is —NR^{AA}R^{BB} or C₁₋₂₀ alkoxy optionally substituted with one or more (e.g., one, two, three, or four) R^{3a}.

In some embodiments, compounds of the disclosure have a structure of formula (I″) or (I″-A), wherein R³ is C₁₋₂₀ alkoxy optionally substituted with one or more (e.g., one, two, three, or four) R^{3a}.

In some embodiments, compounds of the disclosure have a structure of formula (I″) or (I′″-A), wherein R^{A} and R^{B} are each independently H or C₁₋₆ alkyl.

In some embodiments, compounds of the disclosure have a structure of formula (I″) or (I″-A), wherein R³ is —NR^{AA}R^{BB}.

In some embodiments, compounds of the disclosure have a structure of formula (I″) or (I″-A), wherein R^{AA} and R^{BB} are independently selected from the group consisting of H, C₁₋₆ alkyl, C₁₋₂₀ alkylene-NR^{c}R^{d}, and —(C₀₋₂₀ alkylene)-(3-10 membered heterocyclyl), wherein the 3-10 membered heterocyclyl is optionally substituted with one or more (e.g., one, two, three, or four) substituents each independently selected from the group consisting of C₁₋₆ alkyl, hydroxy, and C₁₋₆ alkoxy.

In some embodiments, compounds of the disclosure have a structure of formula (I″) or (I″-A), wherein R^{AA} and R^{BB} are independently selected from the group consisting of H, C₁₋₆ alkyl, C₁₋₂₀ alkylene-NR^{c}R^{d}, and —(C₁₋₂₀ alkylene)-(3-5 membered heterocyclyl), wherein the 3-5 membered heterocyclyl is optionally substituted with C₁₋₆ alkyl.

In some embodiments, compounds of the disclosure have a structure of formula (I″) or (I″-A), wherein each R^{e} and R^{d} are independently, H or C₁₋₆ alkyl.

In some embodiments, compounds of the disclosure have a structure of formula (I′), (I″), or (I″-A), wherein R³ is C₁₋₆ alkoxy optionally substituted with one or more (e.g., one, two, three, or four) R^{3a}.

In some embodiments, compounds of the disclosure have a structure of formula (I′), (I″), or (I″-A), wherein R³ is —(C₀₋₆alkylene)-O-(3-8 membered heterocyclyl optionally substituted with one or more (e.g., one, two, three, or four) substituents each independently selected from the group consisting of C₁₋₆ alkyl, benzyl and —C(O)O—C₁₋₆ alkyl).

In some embodiments, compounds of the disclosure have a structure of formula (I″) or (I″-A), wherein R³ is —(C₀₋₆alkylene)-O-(3-9 membered heterocyclyl optionally substituted with one or more (e.g., one, two, three, or four) substituents each independently selected from the group consisting of C₁₋₆ alkyl, benzyl and —C(O)O—C₁₋₆ alkyl).

In some embodiments, compounds of the disclosure have a structure of formula (I′), (I″), or (I″-A), wherein R⁴ is selected from the group consisting of H, C₁₋₆ alkyl, and halo.

In some embodiments, compounds of the disclosure have a structure of formula (I″) or (I″-A), wherein R⁴ is selected from the group consisting of H, C₁₋₆ alkyl, C₂₋₆ alkynyl optionally substituted with phenyl or 5-6 membered heteroaryl, and halo.

In some embodiments, compounds of the disclosure have a structure of formula (I″) or (I″-A), wherein R⁴ is selected from the group consisting of H, hydroxy, —NR^{e}R^{f}, C₁₋₆ alkyl, C₁₋₆ alkoxy, and C₁₋₆ haloalkyl. In some embodiments, compounds of the disclosure have a structure of formula (I″) of (I″-A), wherein R⁴ is selected from the group consisting of H, hydroxy, —NH₂, —NH(C₁₋₆ alkyl), —N(C₁₋₆ alkyl)₂, C₁₋₆ alkyl, C₁₋₆ alkoxy, and C₁₋₆ haloalkyl.

In some embodiments, compounds of the disclosure have a structure of formula (I′), (I″), or (I″-A), wherein R⁴ is H.

In some embodiments, compounds of the disclosure have a structure of formula (I′), (I″), or (I″-A), wherein R³ and R⁴, together with the atoms to which they are attached, combine to form an aromatic or non-aromatic 5-10 membered monocyclic or bicyclic ring fused to the phenyl, wherein the 5-10 membered ring comprises at least one heteroatom, wherein the 5-10 membered ring is optionally substituted with one or more (e.g., one, two, three, or four) substituents each independently selected from the group consisting of C₁₋₆ alkyl, C₃₋₇ cycloalkyl, phenyl, 5-6 membered heteroaryl, —(C₀₋₆ alkylene)-C(O)NR^{e}R^{f}, —C(O)—C₁₋₆heteroalkyl, —(C₀₋₆ alkylene)-NR^{c}R^{d}, —(C₀₋₆ alkylene)-C₁₋₆ alkoxy, —(C₀₋₆ alkylene)-OH, oxo, —C(O)OH, and —(C₀₋₆ alkylene)-3-7 membered heterocyclyl optionally substituted with C₁₋₆alkyl.

In some embodiments, compounds of the disclosure have a structure of formula (I′), (I″), or (I″-A), wherein R⁵ is H or C₁₋₆ alkyl. In some embodiments, compounds of the disclosure have a structure of formula (I′), (I″), or (I″-A), wherein R⁵ is H. In some embodiments, compounds of the disclosure have a structure of formula (I′), (I′″), or (I″-A), wherein R⁵ is C₁₋₆ alkyl.

In some embodiments, compounds of the disclosure have a structure of formula (I′), (I″), or (I″-A), wherein R⁶ is H or C₁₋₆ alkyl. In some embodiments, compounds of the disclosure have a structure of formula (I′), (I″), or (I″-A), wherein R⁶ is H. In some embodiments, compounds of the disclosure have a structure of formula (I′), (I″), or (I″-A), wherein R⁶ is C₁₋₆ alkyl.

In some embodiments, compounds of the disclosure have a structure of formula (I′), (I″), or (I″-A), wherein each R^{3a} is independently selected from the group consisting of —NR^{A}R^{B}, C₁₋₆ alkoxy, hydroxy, 3-8 membered heterocyclyl, and —(C₀₋₆ alkylene)-(C₃₋₆ cycloalkyl optionally substituted with NR^{c}R^{d}), wherein the 3-8 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, benzyl, C₁₋₆ alkylene-OH, and C₁₋₆ alkylene-C₁₋₆ alkoxy.

In some embodiments, compounds of the disclosure have a structure of formula (I″) or (I″-A), wherein each R^{3a} is independently selected from the group consisting of D, —NR^{A}R^{B}, C₁₋₆ alkoxy, hydroxy, 3-10 membered heterocyclyl, and —(C₀₋₆ alkylene)-(C₃₋₆ cycloalkyl optionally substituted with NR^{c}R^{d}), wherein the 3-10 membered heterocyclyl is optionally substituted with one or more (e.g., one, two, three, or four) substituents each independently selected from the group consisting of halo, hydroxy, C₁₋₆ alkyl optionally substituted with one or more (e.g., one, two, three, or four) D, C₁₋₆ haloalkyl, —C(O)OC₁₋₆ alkyl, C₁₋₆ alkyelene-C₃₋₇ cycloalkyl, benzyl, C₁₋₆ alkylene-OH, and C₁₋₆ alkylene-C₁₋₆ alkoxy.

In some embodiments, compounds of the disclosure have a structure of formula (I′), (I″), or (I″-A), wherein each R^{3a} is —NR^{A}R^{B}. In some embodiments, compounds of the disclosure have a structure of formula (I′), (I″), or (I″-A), wherein each R^{3a} is 3-8 membered heterocyclyl, wherein the 3-8 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, benzyl, C₁₋₆ alkylene-OH, and C₁₋₆ alkylene-C₁₋₆ alkoxy.

In some embodiments, compounds of the disclosure have a structure of formula (I″) or (I″-A), wherein each R^{3a} is 3-10 membered heterocyclyl, wherein the 3-10 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, benzyl, C₁₋₆ alkylene-OH, —C(O)O—C₁₋₆ alkyl, and C₁₋₆ alkylene-C₁₋₆ alkoxy.

In some embodiments, compounds of the disclosure have a structure of formula (I′), (I″), or (I″-A), wherein each R^{A} and R^{B} are independently, for each occurrence, selected from the group consisting of H, C₁₋₆ alkyl, C₁₋₆ alkoxy, C₁₋₆ alkylene-NR^{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy.

In some embodiments, compounds of the disclosure have a structure of formula (I′), (I″), or (I″-A), wherein each R^{A} and R^{B} are independently, for each occurrence, selected from the group consisting of H, C₁₋₆ alkyl, C₁₋₆ alkoxy, and C₁₋₆ alkylene-NR^{c}R^{d}.

In some embodiments, compounds of the disclosure have a structure of formula (I′), (I″), or (I″-A), wherein each R^{A} and R^{B} are independently, for each occurrence, H or C₁₋₆ alkyl.

In some embodiments, compounds of the disclosure have a structure of formula (I′), (I″), or (I″-A), wherein each R^{e} and R^{d} are independently, for each occurrence, selected from the group consisting of H, C₁₋₆ alkyl, benzyl, and —C(O)OC₁₋₆ alkyl.

In some embodiments, compounds of the disclosure have a structure of formula (I′), (I″), or (I″-A), wherein each R^{e} and R^{d} are independently, for each occurrence, H or C₁₋₆ alkyl.

In some embodiments, compounds of the disclosure have a structure of formula (I′), (I″), or (I″-A), wherein each R^{e} and R^{d} are H.

In some embodiments, compounds of the disclosure have a structure of formula (I′), (I″), or (I″-A), wherein each R^{e} and R^{f} are H.

In some embodiments, compounds of the disclosure have a structure of formula (I′), (I″), or (I″-A), wherein each of R^{g} and R^{h} are H.

In some embodiments, compounds of the disclosure have a structure of formula (I′), (I″), or (I″-A), wherein Rⁱ is H.

In some embodiments, compounds of the disclosure have a structure of formula (I″) or (I″-A), wherein R^{x} is C₁₋₆ alkyl. In some embodiments, compounds of the disclosure have a structure of formula (I″) or (I″-A), wherein R^{x} is halo. In some embodiments, compounds of the disclosure have a structure of formula (I″) or (I″-A), wherein R^{x} is D.

In another aspect, provided herein are compounds of formula (I-1″),

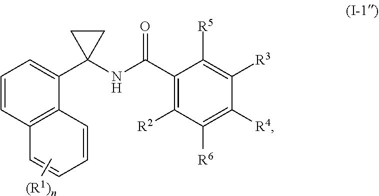

or a pharmaceutically acceptable salt thereof, wherein:
each R¹ is independently selected from the group consisting of C₁₋₆ alkyl, C₁₋₆ haloalkyl, C₁₋₆ haloalkoxy, halo, and —CN;
n is 0, 1, or 2;
each of R², R³, R⁴, and R⁶ is independently selected from the group consisting of hydrogen, C₁₋₆ alkyl, C₁₋₆ haloalkyl, C₁₋₆ haloalkoxy, halo, and —CN; and
R⁵ is C₁₋₆ alkylene-C(O)NH₂, wherein each hydrogen of NH₂ is optionally substituted.

In some embodiments, compounds of the disclosure have a structure of formula (I-1′), wherein n is 0.

In some embodiments, compounds of the disclosure have a structure of formula (I-1′), wherein R², R³, R⁴, and R⁶ are hydrogen.

In some embodiments, compounds of the disclosure have a structure of formula (I-1′), wherein R⁵ is C₁₋₆ alkylene-C(O)NH—NH—C(O)—C₂₋₆alkenylene-C(O)O—C₁₋₆ alkyl.

In some embodiments, compounds of the disclosure have a structure of formula (I-1′), wherein R⁵ is C₂ alkylene-C(O)NH—NH—C(O)-Czalkenylene-C(O)OCH₃.

In another aspect, provided herein are compounds of formula (II′),

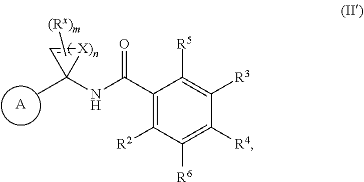

or a pharmaceutically acceptable salt thereof,
wherein
Ring A is phenyl optionally substituted with one or more (e.g., one, two, three, or four) R¹;
each X is independently selected from the group consisting of CH², CH, NH, and O (any hydrogen atom of the NH₂, CH, NRₕ (when Rₕ=H) may be substituted with Rₓ);
each R^{x} is independently C₁₋₆ alkyl or halo when R^{m} is substituted on a carbon atom or each R^{x} is independently C₁₋₆ alkyl when R^{x} is substituted on a nitrogen atom;
m is 0, 1, or 2;
[symbol] is a single bond or double bond;
n is 1 or 2;
each R¹ is independently selected from the group consisting of phenyl, 5-6 membered heteroaryl, C₃₋₇ carbocyclyl, 3-7 membered heterocyclyl, C₁₋₆ haloalkyl, halo, C₁₋₆ alkyl, C₁₋₆ alkoxy, —O—(C₀₋₆ alkylene)-phenyl, —S—C₁₋₆alkyl, —S—C₁₋₆ haloalkyl, —NR^{e}R^{f}, and hydroxy, wherein the phenyl and 5-6 membered heteroaryl are optionally substituted with one or more (e.g., one, two, three, or four) substituents each selected from the group consisting of halo, C₁₋₆ alkyl optionally substituted with 3-7 membered heterocyclyl, C₁₋₆ alkylene-NR^{A}R^{B}, C₁₋₆ haloalkyl, and C₁₋₆ alkoxy;
R² is selected from the group consisting of halo, C₁₋₆ alkyl, C₁₋₆ haloalkyl, C₁₋₆ alkoxy, and C₃₋₆ cycloalkyl;
R³ is selected from the group consisting of C₁₋₆ alkoxy, hydroxy, 3-8 membered heterocyclyl optionally substituted with C₁₋₆alkyl, —O—C₃₋₇ cycloalkyl optionally substituted with —NR^{c}R^{d}, —O-3-8 membered heterocyclyl optionally substituted with —C(O)O—C₁₋₆ alkyl, C₁₋₆ alkyl optionally substituted with —NR^{c}R^{d}, —O-5-6 membered heteroaryl, C₀₋₆ alkylene-CN, C₀₋₆ alkylene-C(O)NR^{e}R^{f}, and —NR^{A}R^{B}, wherein the C₁₋₆ alkoxy is optionally substituted with one or more (e.g., one, two, three, or four) R^{3a},
R⁴ is selected from the group consisting of H, C₁₋₆ alkyl, halo, hydroxy, and —NR^{e}R^{f}; or
R₃ and R₄, together with the atoms to which they are attached, combine to form an aromatic or non-aromatic 5-10 membered monocyclic or bicyclic ring fused to the phenyl, wherein the 5-10 membered ring comprises at least one heteroatom, wherein the 5-10 membered ring is optionally substituted with one or more (e.g., one, two, three, or four) substituents each independently selected from the group consisting of C₁₋₆ alkyl, C₃₋₇ cycloalkyl, phenyl, 5-6 membered heteroaryl, —(C₀₋₆ alkylene)-C(O)NR^{e}R^{f}, —C(O)—C₁₋₆heteroalkyl, —(C₀₋₆ alkylene)-NR^{c}R^{d}, —(C₀₋₆ alkylene)-C₁₋₆ alkoxy, —(C₀₋₆ alkylene)-OH, oxo, —C(O)OH, and —(C₀₋₆ alkylene)-3-7 membered heterocyclyl optionally substituted with C₁₋₆alkyl;
R⁵ is selected from the group consisting of H, halo, and C₁₋₆ alkyl;
R⁶ is selected from the group consisting of H, halo, and C₁₋₆ alkyl;

each R^{3a} is independently selected from the group consisting of C₁₋₆ alkyl, —NR^{A}R^{B}, C₁₋₆ alkoxy, hydroxy, —C(O)NR^{c}R^{d}, 3-8 membered heterocyclyl, phenyl, and —(C₀₋₆ alkylene)-(C₃₋₆ cycloalkyl optionally substituted with NR^{c}R^{d}), wherein the 3-8 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, benzyl, C₁₋₆ alkylene-OH, and C₁₋₆ alkylene-C₁₋₆ alkoxy,
each R^{A} and R^{B} are independently, for each occurrence, selected from the group consisting of H, C₁₋₆ alkyl, C₁₋₆ alkoxy, S(O)₂C₁₋₆ alkyl, —C(O)—C₁₋₆ alkyl, C₁₋₆ alkylene-NR^{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy;
each R^{c} and R^{d} are independently, for each occurrence, selected from the group consisting of H, C₁₋₆ alkyl, benzyl, and —C(O)OC₁₋₆ alkyl, or
R^{c} and R^{d} can be taken together with the nitrogen atom to which they are attached to form a 3-7 membered heterocyclyl; and
each R^{e} and R^{f} are independently, for each occurrence, H or C₁₋₆ alkyl.

In another aspect, provided herein are compounds of formula (II″),

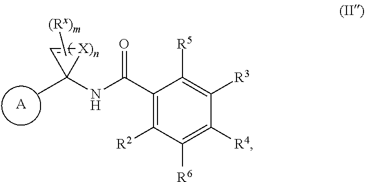

or a pharmaceutically acceptable salt thereof, wherein
Ring A is phenyl optionally substituted with one or more (e.g., one, two, three, or four) R¹;
each X is independently selected from the group consisting of CH₂, CH, NH, and O (any hydrogen atom of the NH₂, CH, NRₕ (when Rₕ=H) may be substituted with Rₓ);
each R^{x} is independently C₁₋₆ alkyl or halo when R^{x} is substituted on a carbon atom or each R^{x} is independently C₁₋₆ alkyl when R^{x} is substituted on a nitrogen atom;
m is 0, 1, or 2;
[symbol] is a single bond or double bond;
n is 1 or 2;
each R¹ is independently selected from the group consisting of phenyl, 5-6 membered heteroaryl, C₃₋₇ carbocyclyl, 3-7 membered heterocyclyl, C₁₋₆ haloalkyl, halo, C₁₋₆ alkyl, C₁₋₆ alkoxy, —O—(C₀₋₆ alkylene)-phenyl, —S—C₁₋₆alkyl, —S—C₁₋₆ haloalkyl, —NR^{c}R^{f}, and hydroxy, wherein the phenyl and 5-6 membered heteroaryl are optionally substituted with one or more (e.g., one, two, three, or four) substituents each selected from the group consisting of halo, C₁₋₆ alkyl optionally substituted with 3-7 membered heterocyclyl, C₁₋₆ alkylene-NR^{A}R^{B}, C₁₋₆ haloalkyl, and C₁₋₆ alkoxy;
R² is selected from the group consisting of halo, C₁₋₆ alkyl, C₁₋₆ haloalkyl, C₁₋₆ alkoxy, and C₃₋₆ cycloalkyl;
R³ is selected from the group consisting of C₁₋₆ alkoxy, 3-8 membered heterocyclyl optionally substituted with C₁₋₆alkyl, —O—C₃₋₇ cycloalkyl optionally substituted with —NR^{c}R^{d}, —O-3-8 membered heterocyclyl optionally substituted with —C(O)O—C₁₋₆ alkyl, C₂₋₆ alkyl optionally substituted with —NR^{c}R^{d}, —O-5-6 membered heteroaryl, C₀₋₆ alkylene-C(O)NR^{e}R^{f}, and —NR^{A}R^{B}, wherein the C₁₋₆ alkoxy is substituted with one or more (e.g., one, two, three, or four) R^{3a}, and wherein R³ is not-N(H) C(O)CH₃ or —NH₂;
R⁴ is selected from the group consisting of H, C₁₋₆ alkyl, halo, hydroxy, and —NR^{e}R^{f}; or
R³ and R⁴, together with the atoms to which they are attached, combine to form an aromatic or non-aromatic 5-10 membered monocyclic or bicyclic ring fused to the phenyl, wherein the 5-10 membered ring comprises at least one heteroatom, wherein the 5-10 membered ring is optionally substituted with one or more (e.g., one, two, three, or four) substituents each independently selected from the group consisting of C₁₋₆ alkyl, C₃₋₇ cycloalkyl, phenyl, 5-6 membered heteroaryl, —(C₀₋₆ alkylene)-C(O)NR^{e}R^{f}, —C(O)—C₁₋₆heteroalkyl, —(C₀₋₆ alkylene)-NR^{c}R^{d}, —(C₀₋₆ alkylene)-C₁₋₆ alkoxy, —(C₀₋₆ alkylene)-OH, oxo, —C(O)OH, and —(C₀₋₆ alkylene)-3-7 membered heterocyclyl optionally substituted with C₁₋₆alkyl;
R⁵ is selected from the group consisting of H, halo, and C₁₋₆ alkyl;
R⁶ is selected from the group consisting of H, halo, and C₁₋₆ alkyl;
each R^{3a} is independently selected from the group consisting of C₁₋₆ alkyl, —NR^{A}R^{B}, C₁₋₆ alkoxy, hydroxy, —C(O)NR^{c}R^{d}, 3-8 membered heterocyclyl, phenyl, and —(C₀₋₆ alkylene)-(C₃₋₆ cycloalkyl optionally substituted with NR^{c}R^{d}), wherein the 3-8 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, benzyl, C₁₋₆ alkylene-OH, and C₁₋₆ alkylene-C₁₋₆ alkoxy,
each R^{A} and R^{B} are independently, for each occurrence, selected from the group consisting of H, C₁₋₆ alkyl, C₁₋₆ alkoxy, S(O)₂C₁₋₆ alkyl, —C(O)—C₁₋₆ alkyl, C₁₋₆ alkylene-NR^{c}R^{d}, 3-6 membered heterocyclyl optionally substituted with oxo or —C(O)O—C₁₋₆ alkyl, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy;
each R^{c} and R^{d} are independently, for each occurrence, selected from the group consisting of H, C₁₋₆ alkyl, benzyl, and —C(O)OC₁₋₆ alkyl, or
R^{c} and R^{d} can be taken together with the nitrogen atom to which they are attached to form a 3-7 membered heterocyclyl; and
each R^{e} and R^{f} are independently, for each occurrence, H or C₁₋₆ alkyl.

In some embodiments, the compound of formula (II′) or (II″) is a compound of formula (IIa′):

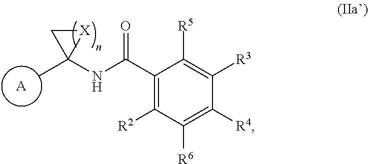

or a pharmaceutically acceptable salt thereof, wherein the variables are as defined above.

In some embodiments, the compound of formula (II′) or (II″) is a compound of formula (IIb′):

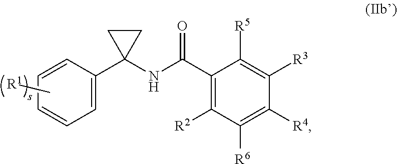

or a pharmaceutically acceptable salt thereof, wherein:
s is 0, 1, 2, or 3; and
R¹-R⁶ are as defined above.

In some embodiments, the compound of formula (II′) or (II″) is a compound of formula (IIc′):

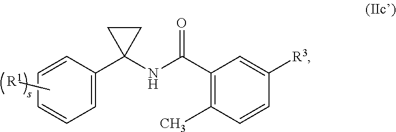

or a pharmaceutically acceptable salt thereof, wherein the variables are as defined above.

In some embodiments, compounds of the disclosure have a structure of formula (II′) or (II″), wherein X is CH₂. In some embodiments, compounds of the disclosure have a structure of formula (II′) or (II″), wherein X is NH. In some embodiments, compounds of the disclosure have a structure of formula (II′) or (II″), wherein X is O.

In some embodiments, compounds of the disclosure have a structure of formula (II′) or (II″), wherein m is 0. In some embodiments, compounds of the disclosure have a structure of formula (II′) or (II″), wherein m is 1. In some embodiments, compounds of the disclosure have a structure of formula (II′) or (II″), wherein m is 2.

In some embodiments, compounds of the disclosure have a structure of formula (II′) or (II″), wherein [symbol] is a single bond. In some embodiments, compounds of the disclosure have a structure of formula (II′) or (II″), wherein [symbol] is a double bond.

In some embodiments, compounds of the disclosure have a structure of formula (II′) or (II″), wherein n is 1. In some embodiments, compounds of the disclosure have a structure of formula (II′) or (II″), wherein n is 2.

In some embodiments, compounds of the disclosure have a structure of formula (II′) or (II″), wherein each R¹ is independently selected from the group consisting of phenyl, 5-6 membered heteroaryl, C₃₋₇ carbocyclyl, C₁₋₆ haloalkyl, halo, C₁₋₆ alkyl, C₁₋₆ alkoxy, —S—C₁₋₆alkyl, —NR^{e}R^{f}, and hydroxy.

In some embodiments, compounds of the disclosure have a structure of formula (II′) or (II″), wherein each R¹ is independently selected from the group consisting of phenyl, 5-6 membered heteroaryl, C₃₋₇ carbocyclyl, C₁₋₆ haloalkyl, halo, C₁₋₆ alkyl, wherein the phenyl and 5-6 membered heteroaryl are optionally substituted with one or more (e.g., one, two, three, or four) substituents each independently selected from the group consisting of halo, C₁₋₆ alkyl, C₁₋₆ alkylene-NR^{A}R^{B}, C₁₋₆ haloalkyl, and C₁₋₆ alkoxy.

In some embodiments, compounds of the disclosure have a structure of formula (II′) or (II″), wherein R² is selected from the group consisting of halo, C₁₋₆ alkyl, C₁₋₆ alkoxy, and C₃₋₆ cycloalkyl. In some embodiments, compounds of the disclosure have a structure of formula (II′) or (II″), wherein R² is halo. In some embodiments, compounds of the disclosure have a structure of formula (II′) or (II″), wherein R² is C₁₋₆ alkyl.

In some embodiments, compounds of the disclosure have a structure of formula (II′) or (II″), wherein R³ is selected from the group consisting of C₁₋₆ alkoxy, hydroxy, 3-8 membered heterocyclyl optionally substituted with C₁₋₆alkyl, —O—C₃₋₇ cycloalkyl optionally substituted with —NR^{c}R^{d}, —O-3-8 membered heterocyclyl optionally substituted with —C(O)O—C₁₋₆ alkyl, and —NR^{A}R^{B}, wherein the C₁₋₆ alkoxy is optionally substituted with one or more (e.g., one, two, three, or four) R^{3a}.

In some embodiments, compounds of the disclosure have a structure of formula (II″), wherein R³ is selected from the group consisting of C₁₋₆ alkoxy, 3-8 membered heterocyclyl optionally substituted with C₁₋₆alkyl, —O—C₃₋₇ cycloalkyl optionally substituted with —NR^{c}R^{d}, —O-3-8 membered heterocyclyl optionally substituted with —C(O)O—C₁₋₆ alkyl, C₂₋₆ alkyl optionally substituted with —NR^{c}R^{d}, —O-5-6 membered heteroaryl, C₀₋₆ alkylene-C(O)NR^{e}R^{f}, and —NR^{A}R^{B}, wherein the C₁₋₆ alkoxy is substituted with one or more (e.g., one, two, three, or four) R^{3a}, and wherein R³ is not-N(H) C(O)CH₃ or —NH₂.

In some embodiments, compounds of the disclosure have a structure of formula (II″), wherein R³ is selected from the group consisting of C₁₋₆ alkoxy, 3-8 membered heterocyclyl optionally substituted with C₁₋₆alkyl, —O—C₃₋₇ cycloalkyl optionally substituted with —NR^{c}R^{d}, —O-(3-8 membered heterocycly) optionally substituted with —C(O)O—C₁₋₆ alkyl, and —NR^{A}R^{B}, wherein the C₁₋₆ alkoxy is optionally substituted with one or more (e.g., one, two, three, or four) R^{3a}.

In some embodiments, compounds of the disclosure have a structure of formula (II′) or (II″), wherein R³ is selected from the group consisting of C₁₋₆ alkoxy and 3-8 membered heterocyclyl optionally substituted with C₁₋₆alkyl, —O—C₃₋₇ cycloalkyl optionally substituted with —NR^{c}R^{d}, wherein the C₁₋₆ alkoxy is optionally substituted with one or more (e.g., one, two, three, or four) R^{3a}.

In some embodiments, compounds of the disclosure have a structure of formula (II′) or (II″), wherein R³ is C₁₋₆ alkoxy optionally substituted with one or more (e.g., one, two, three, or four) R^{3a}.

In some embodiments, compounds of the disclosure have a structure of formula (II′) or (II″), wherein R³ is 3-8 membered heterocyclyl optionally substituted with C₁₋₆alkyl.

In some embodiments, compounds of the disclosure have a structure of formula (II′) or (II″), wherein R⁴ is selected from the group consisting of H, C₁₋₆ alkyl, and halo.

In some embodiments, compounds of the disclosure have a structure of formula (II′) or (II″), wherein R⁴ is H.

In some embodiments, compounds of the disclosure have a structure of formula (II′) or (II″), wherein R³ and R⁴, together with the atoms to which they are attached, combine to form an aromatic or non-aromatic 5-10 membered monocyclic or bicyclic ring fused to the phenyl, wherein the 5-10 membered ring comprises at least one heteroatom, wherein the 5-10 membered ring is optionally substituted with one or more (e.g., one, two, three, or four) substituents each independently selected from the group consisting of C₁₋₆ alkyl, C₃₋₇ cycloalkyl, phenyl, 5-6 membered heteroaryl, —(C₀₋₆ alkylene)-C(O)NR^{e}R^{f}, —C(O)—C₁₋₆heteroalkyl, —(C₀₋₆ alkylene)-NR^{c}R^{d}, —(C₀₋₆ alkylene)-C₁₋₆ alkoxy, —(C₀₋₆ alkylene)-OH, oxo, —C(O)OH, and —(C₀₋₆ alkylene)-3-7 membered heterocyclyl optionally substituted with C₁₋₆alkyl.

In some embodiments, compounds of the disclosure have a structure of formula (II′) or (II″), wherein R⁵ is H or C₁₋₆ alkyl. In some embodiments, compounds of the disclosure have a structure of formula (II′) or (II″), wherein R⁵ is H. In some embodiments, compounds of the disclosure have a structure of formula (II′) or (II″), wherein R⁵ is C₁₋₆ alkyl.

In some embodiments, compounds of the disclosure have a structure of formula (II′) or (II′″), wherein R⁶ is H or C₁₋₆ alkyl. In some embodiments, compounds of the disclosure have a structure of formula (II′) or (II″), wherein R⁶ is H. In some embodiments, compounds of the disclosure have a structure of formula (II′) or (II″), wherein R⁶ is C₁₋₆ alkyl.

In some embodiments, compounds of the disclosure have a structure of formula (II′) or (II″), wherein each R^{3a} is independently selected from the group consisting of —NR^{A}R^{B}, C₁₋₆ alkoxy, hydroxy, 3-8 membered heterocyclyl, and —(C₀₋₆ alkylene)-(C₃₋₆ cycloalkyl optionally substituted with NR^{c}R^{d}), wherein the 3-8 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, benzyl, C₁₋₆ alkylene-OH, and C₁₋₆ alkylene-C₁₋₆ alkoxy.

In some embodiments, compounds of the disclosure have a structure of formula (II′) or (II″), wherein each R^{3a} is —NR^{A}R^{B}.

In some embodiments, compounds of the disclosure have a structure of formula (II′) or (II″), wherein each R^{3a} is 3-8 membered heterocyclyl, wherein the 3-8 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, benzyl, C₁₋₆ alkylene-OH, and C₁₋₆ alkylene-C₁₋₆ alkoxy.

In some embodiments, compounds of the disclosure have a structure of formula (II′) or (II″), wherein each R^{A} and R^{B} are independently, for each occurrence, selected from the group consisting of H, C₁₋₆ alkyl, C₁₋₆ alkoxy, C₁₋₆ alkylene-NR^{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy.

In some embodiments, compounds of the disclosure have a structure of formula (II″), wherein each R^{A} and R^{B} are independently, for each occurrence, selected from the group consisting of H, C₁₋₆ alkyl, C₁₋₆ alkoxy, C₁₋₆ alkylene-NR^{c}R^{d}, 3-6 membered heterocyclyl optionally substituted with oxo or —C(O)O—C₁₋₆ alkyl, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy.

In some embodiments, compounds of the disclosure have a structure of formula (II′) or (II″), wherein each R^{A} and R^{B} are independently, for each occurrence, selected from the group consisting of H, C₁₋₆ alkyl, C₁₋₆ alkoxy, and C₁₋₆ alkylene-NR^{c}R^{d}.

In some embodiments, compounds of the disclosure have a structure of formula (II′) or (II″), wherein each R^{A} and R^{B} are independently, for each occurrence, H or C₁₋₆ alkyl.

In some embodiments, compounds of the disclosure have a structure of formula (II′) or (II″), wherein each R^{c} and R^{d} are independently, for each occurrence, selected from the group consisting of H, C₁₋₆ alkyl, benzyl, and —C(O)OC₁₋₆ alkyl.

In some embodiments, compounds of the disclosure have a structure of formula (II′) or (II″), wherein each R^{e} and R^{d} are independently, for each occurrence, H or C₁₋₆ alkyl.

In some embodiments, compounds of the disclosure have a structure of formula (II′) or (II″), wherein each R^{e} and R^{d} are H.

In some embodiments, compounds of the disclosure have a structure of formula (II′) or (II″), wherein each R^{e} and R^{f} are H.

In another aspect, provided herein are compounds of formula (III″),

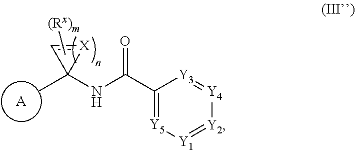

or a pharmaceutically acceptable salt thereof, wherein:
Ring A is selected from naphthyl and 8-12 membered bicyclic heteroaryl, wherein the naphthyl and 8-12 membered bicyclic are optionally substituted with one or more (e.g., one, two, three, or four) R¹;
Y₁ is N or CR⁶;
Y₂ is N or CR⁴;
Y₃ is N or CR⁵;
Y₄ is N or CR³;
Y₅ is N or CR₂;
each X is independently selected from the group consisting of CH₂, CH, NR^{h}, and O (any hydrogen atom of the NH₂, CH, NRₕ (when Rₕ=H) may be substituted with Rₓ), wherein each R^{h} is independently H or C₁₋₆ alkyl;
[symbol] is a single bond or double bond; each R′ is independently C₁₋₆ alkyl or halo;
n is 1 or 2;
m is 0, 1, or 2;
each R¹ is independently selected from the group consisting of C₁₋₆ alkyl, C₁₋₆ haloalkyl, C₁₋₆ haloalkoxy, halo, and —CN;
R² is selected from the group consisting of halo, C₁₋₆ alkyl, C₁₋₆ haloalkyl, and C₁₋₆ alkoxy;
R³ is selected from the group consisting of C₁₋₆ alkyl, C₁₋₆ alkoxy, and hydroxy, wherein the C₁₋₆ alkoxy is optionally substituted with one or more (e.g., one, two, three, or four)-NR^{A}R^{B} or 3-8 membered heterocyclyl optionally substituted with C₁₋₆alkyl;
R⁴ is selected from the group consisting of H, C₁₋₆ alkyl, halo, and hydroxy;
R⁵ is selected from the group consisting of H, halo, and C₁₋₆ alkyl;
R⁶ is selected from the group consisting of H, halo, and C₁₋₆ alkyl; and
each R^{A} and R^{B} are independently, for each occurrence, selected from the group consisting of H, C₁₋₆ alkyl, and C₁₋₆ haloalkyl.

In some embodiments, compounds of the disclosure have a structure of formula (III″), wherein Ring A is naphthyl. In some embodiments, compounds of the disclosure have a structure of formula (III″), wherein Ring A is

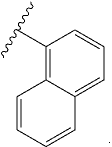 [SMILES: *c1cccc2ccccc12]

In some embodiments, compounds of the disclosure have a structure of formula (III″), wherein at least one of Y¹, Y², Y³, Y⁴, and Y⁵ are N. In some embodiments, Y¹ is N.

In some embodiments, compounds of the disclosure have a structure of formula (III″), wherein Y², Y³, Y⁴, and Y⁵ are not N. In some embodiments, Y² is N.

In some embodiments, compounds of the disclosure have a structure of formula (III″), wherein Y¹, Y³, Y⁴, and Y⁵ are not N. In some embodiments, Y³ is N.

In some embodiments, compounds of the disclosure have a structure of formula (III″), wherein Y¹, Y², Y⁴, and Y⁵ are not N. In some embodiments, Y² and Y³ are N.

In some embodiments, compounds of the disclosure have a structure of formula (III″), wherein Y¹, Y⁴, and Y⁵ are not N.

In some embodiments, compounds of the disclosure have a structure of formula (III″), wherein X is CH₂.

In some embodiments, compounds of the disclosure have a structure of formula (III″), wherein n is 1.

In some embodiments, compounds of the disclosure have a structure of formula (III″), wherein m is 0.

In some embodiments, compounds of the disclosure have a structure of formula (III″), wherein R² is C₁₋₆ alkyl, e.g., methyl.

In some embodiments, compounds of the disclosure have a structure of formula (III″), wherein R³ is C₁₋₆ alkoxy optionally substituted with one or more (e.g., one, two, three, or four)—NR^{A}R^{B}, e.g., —O—(CH₂)₂—N(CH₃)₂.

In some embodiments, compounds of the disclosure have a structure of formula (III″), wherein R³ is C₁₋₆ alkoxy optionally substituted with 3-8 membered heterocyclyl optionally substituted with C₁₋₆alkyl.

In some embodiments, compounds of the disclosure have a structure of formula (III″), wherein R⁴ is H.

In some embodiments, compounds of the disclosure have a structure of formula (III″), wherein R⁵ is H.

In another aspect, provided herein are compounds of formula (IV″),

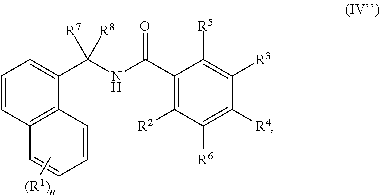

or a stereoisomer or a pharmaceutically acceptable salt thereof, wherein:
each R¹ is independently selected from the group consisting of C₁₋₆ haloalkyl, C₁₋₆ haloalkoxy, halo, —CN, and C₁₋₆ alkyl;
n is 0, 1, or 2;
(A) R² is C₁₋₆ alkyl or halo;
   R³ is selected from the group consisting of C₁₋₆ alkoxy, hydroxy, —C(O)—C₁₋₆alkyl; —O-(3-8 membered oxygen-containing heterocyclyl optionally substituted with one or more (e.g., one, two, three, or four) C₁₋₆ alkyl, —NR^{A}R^{B}, and nitro, wherein the C₁₋₆ alkoxy is optionally substituted with one or more (e.g., one, two, three, or four) R^{3a}, and wherein if R³ is hydroxy, nitro, NH₂, NHCH₃, or N(CH₃)₂, neither R⁷ nor R⁸ is methyl;
   R⁴ is H, C₁₋₆ alkyl, halo, and hydroxy; or
   (B) R² is selected from the group consisting of H, C₁₋₆ alkyl, C₁₋₆ haloalkyl, and C₁₋₆ alkoxy;
   R³ and R⁴, together with the atoms to which they are attached, combine to form:
   i) an aromatic or non-aromatic 5 membered monocyclic ring fused to the phenyl, wherein the 5-membered ring has at least one nitrogen connected to the phenyl at the R³ position or two oxygens, or
   ii) an aromatic or non-aromatic 6 membered monocyclic ring fused to the phenyl, wherein the 6 membered ring has one and only one nitrogen, the nitrogen connected to the phenyl at the R³ position, and the remaining atoms in the ring are carbon,
wherein the 5 or 6 membered ring is optionally substituted with one or more (e.g., one, two, three, or four) substituents each independently selected from the group consisting of oxo, C₁₋₆ alkyl optionally substituted with hydroxy or —NR^{c}R^{d}, —(C₀₋₆ alkylene)-3-7 membered heterocyclyl optionally substituted with C₁₋₆alkyl;
R⁵ is selected from the group consisting of H, halo, and C₁₋₆ alkyl;
R⁶ is selected from the group consisting of H, halo, and C₁₋₆ alkyl;
R⁷ is selected from the group consisting of hydrogen, C₁₋₆ alkyl optionally substituted with C₁₋₆ alkoxy, C₂₋₆ alkynyl, C₁₋₆ haloalkyl, cyano, and 3-8 membered heterocyclyl optionally substituted with C₁₋₆ alkyl;
R⁸ is hydrogen or C₁₋₆ alkyl;
each R^{3a} is independently selected from the group consisting of C₁₋₆ alkyl, —NR^{e}R^{f}, and 3-8 membered heterocyclyl, wherein the 3-8 membered heterocyclyl is optionally substituted with one or more (e.g., one, two, three, or four) substituents each independently selected from the group consisting of halo, hydroxy, C₁₋₆ alkyl, and C₁₋₆ haloalkyl;
each R^{A} and R^{B} are independently, for each occurrence, selected from the group consisting of H, C₁₋₆ alkyl, C₁₋₆ haloalkyl, C₁₋₆ alkoxy, S(O)₂C₁₋₂ alkyl, —C(O)-unsubstituted C₁₋₆ alkyl, 3-6 membered unsaturated heterocyclyl optionally substituted with one oxo, 5-6 membered heteroaryl substituted with C₁₋₆ alkyl or C₁₋₆ alkoxy, and pyrazolyl, wherein the C₁₋₆ alkyl is optionally substituted with one or more (e.g., one, two, three, or four) hydroxy and/or one NH₂, wherein if one of R^{A} and R^{B} is —C(O)-unsubstituted C₁₋₆ alkyl, the other of R^{A} and R^{B} is not H, and wherein R^{A} and R^{B} are not-(CH₂)₂NH₂ or —CH₂C(CH₃)₂NH₂;
each R^{e} and R^{d} are independently, for each occurrence, selected from the group consisting of H, C₁₋₆ alkyl, and —C(O)OC₁₋₆ alkyl, and
each R^{e} and R^{f} are independently, for each occurrence, selected from the group consisting of H, C₁₋₆ alkyl, and —C(O)OC₁₋₆alkyl,
   wherein the compound is not a compound represented by:

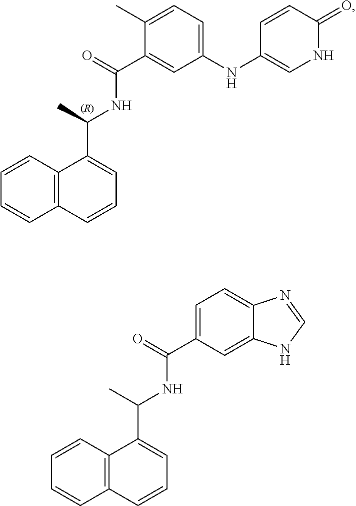

or a stereoisomer or a pharmaceutically acceptable salt thereof.

In some embodiments, compounds of the disclosure have a structure of formula (IV″), wherein n is 0.

In some embodiments, compounds of the disclosure have a structure of formula (IV″), wherein R² is C₁₋₆ alkyl. In some embodiments, compounds of the disclosure have a structure of formula (IV″), wherein R² is methyl. In some embodiments, compounds of the disclosure have a structure of formula (IV″), wherein R² is halo. In some embodiments, compounds of the disclosure have a structure of formula (IV″), wherein R² is hydrogen.

In some embodiments, compounds of the disclosure have a structure of formula (IV″), wherein R³ is selected from the group consisting of C₁₋₆ alkoxy, hydroxy, —C(O)—C₁₋₆alkyl; —O-(3-8 membered oxygen-containing heterocyclyl optionally substituted with one or more (e.g., one, two, three, or four) C₁₋₆ alkyl, —NR^{A}R^{B}, and nitro, wherein the C₁₋₆ alkoxy is optionally substituted with one or more (e.g., one, two, three, or four) R^{3a}, and wherein if R³ is hydroxy, nitro, NH₂, NHCH₃, or N(CH₃)₂, R⁷ is not methyl; and R⁴ is H, C₁₋₆ alkyl, halo, and hydroxy.

In some embodiments, compounds of the disclosure have a structure of formula (IV″), wherein R³ is selected from the group consisting of C₁₋₆ alkoxy, hydroxy, —O-(3-8 membered oxygen-containing heterocyclyl optionally substituted with one or more (e.g., one, two, three, or four) C₁₋₆ alkyl, —NR^{A}R^{B}, and nitro, wherein the C₁₋₆ alkoxy is optionally substituted with one or more (e.g., one, two, three, or four) R^{3a}, and wherein if R³ is hydroxy, nitro, NH₂, NHCH₃, or N(CH₃)₂, R⁷ is not methyl; and R⁴ is H, C₁₋₆ alkyl, halo, and hydroxy.

In some embodiments, compounds of the disclosure have a structure of formula (IV″), wherein R³ is C₁₋₆ alkoxy optionally substituted with one or more (e.g., one, two, three, or four) R^{3a}.

In some embodiments, compounds of the disclosure have a structure of formula (IV″), wherein R³ is hydroxy. In some embodiments, compounds of the disclosure have a structure of formula (IV″), wherein R³ is —C(O)—C₁₋₆alkyl.

In some embodiments, compounds of the disclosure have a structure of formula (IV″), wherein R³ is —O-(3-8 membered oxygen-containing heterocyclyl optionally substituted with one or more (e.g., one, two, three, or four) C₁₋₆ alkyl.

In some embodiments, compounds of the disclosure have a structure of formula (IV″), wherein R³ is —NR^{A}R^{B}. In some embodiments, compounds of the disclosure have a structure of formula (IV″), wherein R³ is and nitro.

In some embodiments, compounds of the disclosure have a structure of formula (IV″), wherein R⁴ is H.

In some embodiments, compounds of the disclosure have a structure of formula (IV″), wherein R⁵ is H.

In some embodiments, compounds of the disclosure have a structure of formula (IV″), wherein R⁶ is H.

In some embodiments, compounds of the disclosure have a structure of formula (IV″), wherein R⁷ is hydrogen. In some embodiments, compounds of the disclosure have a structure of formula (IV″), wherein R⁷ is C₁₋₆ alkyl optionally substituted with C₁₋₆ alkoxy. In some embodiments, compounds of the disclosure have a structure of formula (IV″), wherein R⁷ is C₂₋₆ alkynyl. In some embodiments, compounds of the disclosure have a structure of formula (IV″), wherein R⁷ is C₁₋₆ haloalkyl. In some embodiments, compounds of the disclosure have a structure of formula (IV′″), wherein R⁷ is cyano. In some embodiments, compounds of the disclosure have a structure of formula (IV″), wherein R⁷ is 3-8 membered heterocyclyl optionally substituted with C₁₋₆ alkyl.

In some embodiments, compounds of the disclosure have a structure of formula (IV″), wherein R⁸ is hydrogen. In some embodiments, compounds of the disclosure have a structure of formula (IV″), wherein R⁸ is C₁₋₆ alkyl.

In some embodiments, compounds of the disclosure have a structure of formula (IV″), wherein R^{3a} is —NR^{e}R^{f}. In some embodiments, compounds of the disclosure have a structure of formula (IV″), wherein R^{3a} is 3-8 membered heterocyclyl optionally substituted with one or more (e.g., one, two, three, or four) substituents each independently selected from the group consisting of halo, hydroxy, C₁₋₆ alkyl, and C₁₋₆ haloalkyl.

In some embodiments, compounds of the disclosure have a structure of formula (IV″), wherein each R^{A} and R^{B} are independently, for each occurrence, selected from the group consisting of H, C₁₋₆ alkyl, S(O)₂C₁₋₂ alkyl, —C(O)-unsubstituted C₁₋₆ alkyl, 3-6 membered unsaturated heterocyclyl optionally substituted with one oxo, 5-6 membered heteroaryl substituted with C₁₋₆ alkyl or C₁₋₆ alkoxy, and pyrazolyl, wherein the C₁₋₆ alkyl is optionally substituted with one or more (e.g., one, two, three, or four) hydroxy and/or one NH₂.

In some embodiments, compounds of the disclosure have a structure of formula (IV″), wherein each R^{A} and R^{B} are independently, for each occurrence, selected from the group consisting of H, C₁₋₆ alkyl, S(O)₂C₁₋₂ alkyl, —C(O)-unsubstituted C₁₋₆ alkyl, 5-6 membered heteroaryl substituted with C₁₋₆ alkyl or C₁₋₆ alkoxy, and pyrazolyl, wherein the C₁₋₆ alkyl is optionally substituted with one or more (e.g., one, two, three, or four) hydroxy and/or one NH₂.

In some embodiments, compounds of the disclosure have a structure of formula (IV″), wherein R^{A} is H and R^{B} is H. In some embodiments, compounds of the disclosure have a structure of formula (IV″), wherein R^{A} is H and R^{B} is C₁₋₆ alkyl optionally substituted with one or more (e.g., one, two, three, or four) hydroxy and/or one NH₂. In some embodiments, compounds of the disclosure have a structure of formula (IV″), wherein R^{A} is H and R^{B} is S(O)₂C₁₋₂ alkyl. In some embodiments, compounds of the disclosure have a structure of formula (IV″), wherein R^{A} is H and R^{B} is 5-6 membered heteroaryl substituted with C₁₋₆ alkyl or C₁₋₆ alkoxy. In some embodiments, compounds of the disclosure have a structure of formula (IV″), wherein each R^{A} is H and R^{B} is pyrazolyl. In some embodiments, compounds of the disclosure have a structure of formula (IV″), wherein each R^{A} is C₁₋₆ alkyl and R^{B} is —C(O)-unsubstituted C₁₋₆ alkyl, wherein the C₁₋₆ alkyl is optionally substituted with one or more (e.g., one, two, three, or four) hydroxy and/or one NH₂.

In some embodiments, compounds of the disclosure have a structure of formula (IV″), wherein R³ and R⁴, together with the atoms to which they are attached, combine to form: an aromatic or non-aromatic 5 membered monocyclic ring fused to the phenyl, wherein the 5-membered ring has at least one nitrogen connected to the phenyl at the R³ position or two oxygens, wherein the 6 membered ring is optionally substituted with one or more (e.g., one, two, three, or four) substituents each independently selected from the group consisting of oxo, C₁₋₆ alkyl optionally substituted with hydroxy or —NR^{c}R^{d}, —(C₀₋₆ alkylene)-3-7 membered heterocyclyl optionally substituted with C₁₋₆alkyl.

In some embodiments, compounds of the disclosure have a structure of formula (IV″), wherein R³ and R⁴, together with the atoms to which they are attached, combine to form: an aromatic or non-aromatic 5 membered monocyclic ring fused to the phenyl, wherein the 5-membered ring has at least one nitrogen connected to the phenyl at the R³ position, wherein the 6 membered ring is optionally substituted with one or more (e.g., one, two, three, or four) substituents each independently selected from the group consisting of oxo, C₁₋₆ alkyl optionally substituted with hydroxy or —NR^{c}R^{d}, —(C₀₋₆ alkylene)-3-7 membered heterocyclyl optionally substituted with C₁₋₆alkyl.

In some embodiments, compounds of the disclosure have a structure of formula (IV″), wherein R³ and R⁴, together with the atoms to which they are attached, combine to form an aromatic or non-aromatic 6 membered monocyclic ring fused to the phenyl, wherein the 6 membered ring has one and only one nitrogen, the nitrogen connected to the phenyl at the R³ position, and the remaining atoms in the ring are carbon, wherein the 6 membered ring is optionally substituted with one or more (e.g., one, two, three, or four) substituents each independently selected from the group consisting of oxo, C₁₋₆ alkyl optionally substituted with hydroxy or —NR^{c}R^{d}, —(C₀₋₆ alkylene)-3-7 membered heterocyclyl optionally substituted with C₁₋₆alkyl.

In another aspect, provided here are compounds of formula (VI″):

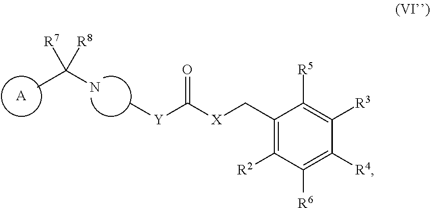

or a stereoisomer or a pharmaceutically acceptable salt thereof, wherein:

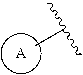

is

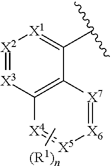

or an optionally substituted phenyl;
X is NR⁹ or a bond;
Y is NR⁹, CH₂, or a bond;
R⁹ is selected from H and C₁₋₆alkyl;

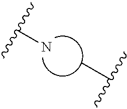

is a nitrogen containing 4-6 membered heterocyclylene or 4-6 membered heterocycle fused to phenyl;
each of X¹, X², X³, X⁴, X⁵, and X⁶ is CH or N, wherein the CH may be substituted with R¹;
wherein
when

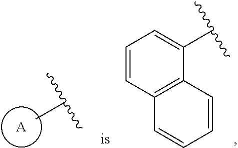

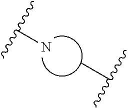

is not a 6-membered monocyclic heterocyclylene;
each R¹ is independently selected from the group consisting of C₁₋₆ haloalkyl, C₁₋₆ haloalkoxy, halo, —CN, C₁₋₆ alkyl, C₁₋₆ alkoxy, —NR^{e}R^{f}, 3-8 membered heterocyclyl, and 5-6 membered heteroaryl;
n is 0, 1, or 2;
each of R², R³, R⁴, R⁵, and R⁶ is independently selected from the group consisting of hydrogen, C₁₋₆ haloalkyl, C₁₋₆ haloalkoxy, halo, —CN, —NR^{e}R^{f}, C₁₋₆ alkoxy, and C₁₋₆ alkyl;
R⁷ is selected from the group consisting of C₁₋₆ alkyl optionally substituted with C₁₋₆ alkoxy, C₂₋₆ alkynyl, C₁₋₆ haloalkyl, cyano, and 3-8 membered heterocyclyl optionally substituted with C₁₋₆ alkyl;
R⁸ is hydrogen; or
R⁷ and R⁸ may be taken together with the carbon to which they are attached to form a 3-4 membered cycloalkyl or heterocyclyl ring; and
each R^{e} and R^{f} are independently, for each occurrence, selected from the group consisting of H, C₁₋₆ alkyl, and —C(O)OC₁₋₆alkyl.

In some embodiments, compounds of the disclosure have a structure of formula (VI″), wherein

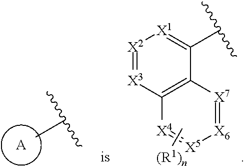

In some embodiments, compounds of the disclosure have a structure of formula (VI″), wherein

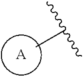

is phenyl optionally substituted with C₁₋₆ alkyl.

In some embodiments, compounds of the disclosure have a structure of formula (VI″), wherein X is NR⁹. In some embodiments, compounds of the disclosure have a structure of formula (VI″), wherein X is a bond.

In some embodiments, compounds of the disclosure have a structure of formula (VI″), wherein Y is NR⁹. In some embodiments, compounds of the disclosure have a structure of formula (VI″), wherein Y is CH₂. In some embodiments, compounds of the disclosure have a structure of formula (VI″), wherein Y is a bond.

In some embodiments, compounds of the disclosure have a structure of formula (VI″), wherein R⁹ is H. In some embodiments, compounds of the disclosure have a structure of formula (VI″), wherein R⁹ is C₁₋₆alkyl.

In some embodiments, compounds of the disclosure have a structure of formula (VI″), wherein

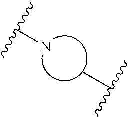

is a nitrogen containing 4-6 membered heterocyclylene, e.g., 4-membered heterocyclylene, 5-membered heterocyclylene, 6-membered heterocyclylene.

In some embodiments, compounds of the disclosure have a structure of formula (VI″), wherein

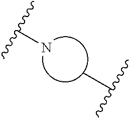

is a nitrogen containing 4-6 membered heterocyclylene, e.g., 4-5 membered heterocyclylene, 4-membered heterocyclylene, 5-membered heterocyclylene, 6-membered heterocyclylene, e.g., 6-membered monocyclic heterocyclylene, 6-membered bicyclic heterocyclylene.

In some embodiments, compounds of the disclosure have a structure of formula (VI″), wherein R¹ is C₁₋₆ alkoxy. In some embodiments, compounds of the disclosure have a structure of formula (VI″), wherein R¹ is 5-6 membered heteroaryl.

In some embodiments, compounds of the disclosure have a structure of formula (VI″), wherein n is 0. In some embodiments, compounds of the disclosure have a structure of formula (VI″), wherein n is 1.

In some embodiments, compounds of the disclosure have a structure of formula (VI″), wherein each of R², R³, R⁴, R⁵, and R⁶ is independently selected from the group consisting of hydrogen and halo.

In some embodiments, compounds of the disclosure have a structure of formula (VI″), wherein R⁷ is C₁₋₆ alkyl optionally substituted with C₁₋₆ alkoxy and R⁸ is hydrogen. In some embodiments, compounds of the disclosure have a structure of formula (VI″), wherein R⁷ is methyl and R⁸ is hydrogen.

In some embodiments, compounds of the disclosure have a structure of formula (VI″), wherein R⁷ and R⁸ are taken together with the carbon to which they are attached to form a 3-4 membered cycloalkyl ring. In some embodiments, compounds of the disclosure have a structure of formula (VI″), wherein R⁷ and R⁸ are taken together with the carbon to which they are attached to form a 3-4 membered heterocyclyl ring.

In another aspect, provided herein are compounds of formula (VII″):

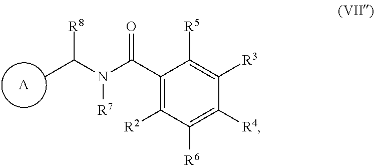

or a stereoisomer or a pharmaceutically acceptable salt thereof, wherein:
ring A is selected from:

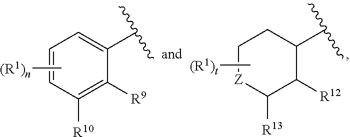

Z is CH₂ or NH, wherein any hydrogen atom of the CH₂ or NH may be substituted with R¹;
each R¹ is independently selected from the group consisting of C₁₋₆ haloalkyl, C₁₋₆ haloalkoxy, halo, —CN, C₁₋₆ alkyl, C₁₋₆ alkoxy, —S(O)ₜC₁₋₆alkyl; —NR^{e}R^{f}, 3-8 membered heterocyclyl, and 5-6 membered heteroaryl;
t is 0, 1, or 2;
n is 0, 1, or 2;
each of R², R³, R⁴, R⁵, and R⁶ is independently selected from the group consisting of hydrogen, C₁₋₆ haloalkyl, C₁₋₆ haloalkoxy, halo, —CN, —NR^{e}R^{f}, C₁₋₆ alkoxy, and C₁₋₆ alkyl; or R³ and R⁴ may be taken together with the atoms to which they are attached to form an aromatic 5-6 membered monocyclic ring fused to the phenyl to which R³ and R⁴ are attached;
wherein the 5-6 membered ring is optionally substituted with one or more (e.g., one, two, three, or four) C₁₋₆ alkyl optionally substituted with hydroxy or —NR^{c}R^{d},
R⁷ is hydrogen or C₁₋₆ alkyl;
R⁸ is hydrogen, C₁₋₆ alkyl, or C₁₋₆haloalkyl;
R⁹ is hydrogen or C₁₋₆ alkyl;
R¹⁰ is (5-6 membered heteroarylene)-(C₀₋₆alkylene)-R¹¹;
R¹¹ is 3-7 membered heterocyclyl or —NR^{g}R^{h}, and
R¹² and R¹³ are hydrogen; or
R⁷ and R⁹ may be taken together with the atoms to which they are attached to form a 5-6 membered heterocycle fused to the phenyl to which R⁹ is attached;
R⁹ and R¹⁰ may be taken together with the atoms to which they are attached to form 6-membered heterocycle or cyclohexane ring fused to the phenyl to which R⁹ and R¹⁰ are attached;
R¹² and R¹³ may be taken together with the atoms to which they are attached to form phenyl ring fused to the ring to which R¹² and R¹³ are attached;
each R^{c} and R^{d} are independently H or C₁₋₆ alkyl; and
each R^{e} and R^{f} are independently selected from the group consisting of H, C₁₋₆ alkyl, C₃₋₆ cycloalkyl, 3-7 membered heterocyclyl, and 5-6 membered heteroaryl, wherein the C₃₋₆ cycloalkyl, 3-7 membered heterocyclyl and 5-6 membered heteroaryl are optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy;
each R^{g} and R^{h} are independently selected from the group consisting of H, C₁₋₆ alkyl, C₃₋₆ cycloalkyl, 3-7 membered heterocyclyl, and 5-6 membered heteroaryl, wherein the C₃₋₆ cycloalkyl, 3-7 membered heterocyclyl and 5-6 membered heteroaryl are optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy, wherein when R^{g} is hydrogen, R^{h} is not hydrogen;
wherein when R¹⁰ is (5-6 membered heteroarylene)-(C₀₋₆alkylene)-(3-7 membered heterocyclyl), and R³ is —NR^{c}R^{d}, R⁸ is hydrogen; and
wherein when R^{g} or R^{h} is C₃₋₆ cycloalkyl or 3-7 membered heterocyclyl and R³ is —NR^{e}R^{f}, R^{e} or R^{f} is not 3-7 membered heterocyclyl.

In some embodiments, compounds of the disclosure have a structure of formula (VII″), wherein ring A is

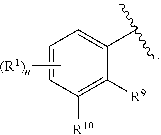

In some embodiments, compounds of the disclosure have a structure of formula (VII″), wherein ring A is

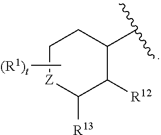

In some embodiments, compounds of the disclosure have a structure of formula (VII″), wherein Z is CH₂ wherein the CH₂ may be substituted with R¹. In some embodiments, compounds of the disclosure have a structure of formula (VII″), wherein Z is NH, wherein the NH may be substituted with R¹.

In some embodiments, compounds of the disclosure have a structure of formula (VII″), wherein t is 0.

In some embodiments, compounds of the disclosure have a structure of formula (VII″), wherein n is 0.

In some embodiments, compounds of the disclosure have a structure of formula (VII″), wherein each of R², R³, R⁴, R⁵, and R⁶ is independently selected from hydrogen and —NR^{e}R^{f}.

In some embodiments, compounds of the disclosure have a structure of formula (VII″), wherein R³ and R⁴ are taken together with the atoms to which they are attached to form an aromatic 5-6 membered monocyclic ring fused to the phenyl to which R³ and R⁴ are attached, wherein the 5-6 membered ring is optionally substituted with one or more (e.g., one, two, three, or four) C₁₋₆ alkyl optionally substituted with hydroxy or —NR^{c}R^{d}.

In some embodiments, compounds of the disclosure have a structure of formula (VII″), wherein R⁷ is hydrogen. In some embodiments, compounds of the disclosure have a structure of formula (VII″), wherein R⁷ is C₁₋₆ alkyl.

In some embodiments, compounds of the disclosure have a structure of formula (VII″), wherein R⁸ is hydrogen. In some embodiments, compounds of the disclosure have a structure of formula (VII″), wherein R⁸ is C₁₋₆ alkyl.

In some embodiments, compounds of the disclosure have a structure of formula (VII″), wherein R⁹ is hydrogen. In some embodiments, compounds of the disclosure have a structure of formula (VII″), wherein R⁹ is C₁₋₆ alkyl.

In some embodiments, compounds of the disclosure have a structure of formula (VII″), wherein R¹¹ is 3-7 membered heterocyclyl. In some embodiments, compounds of the disclosure have a structure of formula (VII″), wherein R¹¹ is —NR^{g}R^{h}.

In some embodiments, compounds of the disclosure have a structure of formula (VII″), wherein R⁷ and R⁹ may be taken together with the atoms to which they are attached to form a 5-6 membered heterocycle fused to the phenyl to which R⁹ is attached.

In some embodiments, compounds of the disclosure have a structure of formula (VII″), wherein R⁹ and R¹⁰ may be taken together with the atoms to which they are attached to form 6-membered heterocycle or cyclohexane ring fused to the phenyl to which R⁹ and R¹⁰ are attached.

In some embodiments, compounds of the disclosure have a structure of formula (VII″), wherein R¹² and R¹³ may be taken together with the atoms to which they are attached to form phenyl ring fused to the ring to which R¹² and R¹³ are attached.

In some embodiments, compounds of the disclosure have a structure of formula (VII″), wherein each R^{e} and R^{f} are independently selected from H and C₃₋₆ cycloalkyl optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy.

In some embodiments, compounds of the disclosure have a structure of formula (VII″), wherein each R^{e} and R^{f} are independently selected from H and 3-7 membered heterocyclyl optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy.

In some embodiments, compounds of the disclosure have a structure of formula (VII″), wherein each R^{e} and R^{f} are independently selected from H and 5-6 membered heteroaryl optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy.

In some embodiments, compounds of the disclosure have a structure of formula (VII″), wherein each R^{e} and R^{f} are independently selected from H and C₁₋₆ alkyl.

In some embodiments, compounds of the disclosure have a structure of formula (VII″), wherein each R^{g} and R^{h} are independently selected from H and C₁₋₆ alkyl.

In some embodiments, compounds of the disclosure have a structure of formula (VII″), wherein each R^{g} and R^{h} are independently selected from H and C₃₋₆ cycloalkyl optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy.

In some embodiments, compounds of the disclosure have a structure of formula (VII″), wherein each R^{g} and R^{h} are independently selected from H and 3-7 membered heterocyclyl optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy.

In some embodiments, compounds of the disclosure have a structure of formula (VII″), wherein each R^{g} and R^{h} are independently selected from the group consisting of H and 5-6 membered heteroaryl optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy.

In another aspect, provided herein are compounds of formula (VIII″)

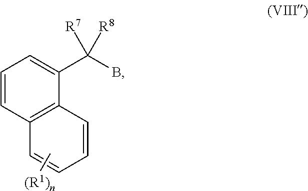

or a stereoisomer or a pharmaceutically acceptable salt thereof; wherein
B is selected from:

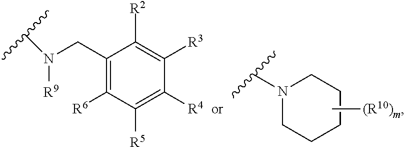

each R¹ is independently selected from the group consisting of C₁₋₆ haloalkyl, C₁₋₆ haloalkoxy, halo, —CN, and C₁₋₆ alkyl;
n is 0, 1, or 2;
each of R², R³, R⁴, R⁵, and R⁶ is independently selected from the group consisting of hydrogen, C₁₋₆ haloalkyl, C₁₋₆ haloalkoxy, halo, —CN, —NR^{e}R^{f}, NO₂, C₁₋₆ alkoxy, and C₁₋₆ alkyl; or
R⁴ and R⁵ may be taken together with the atoms to which they are attached to form 5-membered heteroaryl fused to the phenyl to which R⁴ and R⁵ are attached;
R⁹ is hydrogen or C₁₋₆ alkyl;
each R¹⁰ is independently selected from the group consisting of C₁₋₆ haloalkyl, C₁₋₆ haloalkoxy, halo, —CN, —NR^{e}R^{f}, NO₂, C₁₋₆ alkoxy, C₁₋₆ alkyl, and C(O)NH(C₁₋₆ alkylene)Ph wherein the Ph is optionally substituted with halogen;
m is 0, 1, or 2;
R⁶ and R⁹ may be taken together with the atoms to which they are attached to form piperidine ring fused to the phenyl to which R⁶ is attached;
each of R⁷ and R⁸ is independently hydrogen, C₁₋₆ alkyl, or C₁₋₆haloalkyl, or R⁷ and R⁸ may be taken together with the atom to which they are attached to form 3-4 membered cycloalkyl or heterocyclyl ring;
wherein when R⁹ is methyl, R⁷ and R⁸ are not methyl;
wherein when R⁹ is hydrogen, at least one of R² and R⁶ is not hydrogen;
each of R^{e} and R^{f} is independently selected from the group consisting of hydrogen, C₁₋₆ alkyl, and —C(O)CH₂Ph.

In some embodiments, compounds of the disclosure have a structure of formula (VIII″), wherein B is

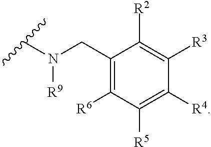

In some embodiments, compounds of the disclosure have a structure of formula (VIII″), wherein B is

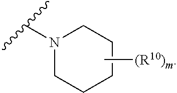

In some embodiments, compounds of the disclosure have a structure of formula (VIII″), wherein n is 0.

In some embodiments, compounds of the disclosure have a structure of formula (VIII″), wherein each of R², R³, R⁴, R⁵, and R⁶ is independently selected from the group consisting of hydrogen, —NR^{e}R^{f}, NO₂, and C₁₋₆ alkyl.

In some embodiments, compounds of the disclosure have a structure of formula (VIII″), wherein R⁴ and R⁵ may be taken together with the atoms to which they are attached to form 5-membered heteroaryl fused to the phenyl to which R⁴ and R⁵ are attached.

In some embodiments, compounds of the disclosure have a structure of formula (VIII″), wherein R⁹ is hydrogen. In some embodiments, compounds of the disclosure have a structure of formula (VIII″), wherein R⁹ is C₁₋₆ alkyl.

In some embodiments, compounds of the disclosure have a structure of formula (VIII″), wherein R¹⁰ is C(O)NH(C₁₋₆ alkylene)Ph wherein the Ph is optionally substituted with halogen.

In some embodiments, compounds of the disclosure have a structure of formula (VIII″), wherein m is 0. In some embodiments, compounds of the disclosure have a structure of formula (VIII″), wherein m is 1.

In some embodiments, compounds of the disclosure have a structure of formula (VIII″), wherein R⁶ and R⁹ are taken together with the atoms to which they are attached to form piperidine ring fused to the phenyl to which R⁶ is attached.

In some embodiments, compounds of the disclosure have a structure of formula (VIII″), wherein each of R⁷ is hydrogen. In some embodiments, compounds of the disclosure have a structure of formula (VIII″), wherein each of R⁷ is C₁₋₆ alkyl.

In some embodiments, compounds of the disclosure have a structure of formula (VIII″), wherein R⁷ and R⁸ are taken together with the atom to which they are attached to form 3-4 membered cycloalkyl or heterocyclyl ring.

In some embodiments, compounds of the disclosure have a structure of formula (VIII″), wherein each of R⁸ is hydrogen. In some embodiments, compounds of the disclosure have a structure of formula (VIII″), wherein each of R⁸ is C₁₋₆ alkyl.

In another aspect, provided herein are compounds of formula (IX″):

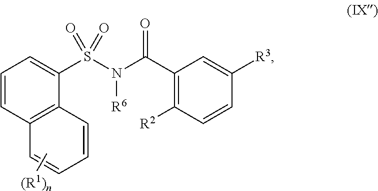

or a stereoisomer or a pharmaceutically acceptable salt thereof; wherein
each R¹ is independently selected from the group consisting of C₁₋₆ haloalkyl, C₁₋₆ haloalkoxy, halo, —CN, and C₁₋₆ alkyl;
n is 0, 1, or 2;
R² is C₁₋₆ alkyl;
R³ is selected from the group consisting of C₁₋₆ haloalkyl, C₁₋₆ haloalkoxy, halo, —CN, —NR^{e}R^{f}, NO₂, C₁₋₆ alkoxy, and C₁₋₆ alkyl;
R⁶ is hydrogen or C₁₋₆ alkyl; and each of R^{e} and R^{f} is independently selected from hydrogen and C₁₋₆ alkyl.

In some embodiments, compounds of the disclosure have a structure of formula (IX″), wherein n is 0.

In some embodiments, compounds of the disclosure have a structure of formula (IX″), wherein R² is methyl.

In some embodiments, compounds of the disclosure have a structure of formula (IX″), wherein R³ is —NR^{e}R^{f}.

In some embodiments, compounds of the disclosure have a structure of formula (IX″), wherein R⁶ is hydrogen. In some embodiments, compounds of the disclosure have a structure of formula (IX″), wherein R⁶ is C₁₋₆ alkyl.

In some embodiments, compounds of the disclosure have a structure of formula (IX″), wherein R^{e} and R^{f} are hydrogen.

In another aspect, provided herein are compounds of formula (X″)

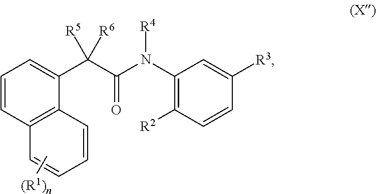

or a stereoisomer or a pharmaceutically acceptable salt thereof; wherein
each R¹ is independently selected from the group consisting of C₁₋₆ haloalkyl, C₁₋₆ haloalkoxy, halo, —CN, and C₁₋₆ alkyl;
n is 0, 1, or 2;
R² is C₁₋₆ alkyl; and
R³ is selected from the group consisting of C₁₋₆ alkyl, C₁₋₆ haloalkyl, C₁₋₆ haloalkoxy, halo, —CN, —NR^{e}R^{f}, NO₂, C₁₋₆ alkoxy optionally substituted with 3-8 membered heterocyclyl or —NR^{e}R^{f}, and
R⁴ is hydrogen or C₁₋₆ alkyl;
R⁵ is hydrogen, C₁₋₆ alkyl, C₁₋₆haloalkyl;
R⁶ is hydrogen or C₁₋₆ alkyl; or
R⁵ and R⁶ may be taken together with the carbon to which they are attached to form 3-4 membered cycloalkyl or heterocycle ring; and
each of R^{e} and R^{f} is independently selected from hydrogen and C₁₋₆ alkyl.

In some embodiments, compounds of the disclosure have a structure of formula (X″), wherein n is 0.

In some embodiments, compounds of the disclosure have a structure of formula (X″), wherein R² is methyl.

In some embodiments, compounds of the disclosure have a structure of formula (X″), wherein R³ is —NR^{e}R^{f}. In some embodiments, compounds of the disclosure have a structure of formula (X″), wherein R³ is C₁₋₆ alkoxy optionally substituted with —NR^{e}R^{f}. In some embodiments, compounds of the disclosure have a structure of formula (X″), wherein R³ is C₁₋₆ alkoxy optionally substituted with 3-8 membered heterocyclyl.

In some embodiments, compounds of the disclosure have a structure of formula (X″), wherein R⁴ is hydrogen. In some embodiments, compounds of the disclosure have a structure of formula (X″), wherein R⁴ is C₁₋₆ alkyl.

In some embodiments, compounds of the disclosure have a structure of formula (X″), wherein R⁵ is hydrogen. In some embodiments, compounds of the disclosure have a structure of formula (X″), wherein R⁵ is C₁₋₆ alkyl.

In some embodiments, compounds of the disclosure have a structure of formula (X″), wherein R⁶ is hydrogen. In some embodiments, compounds of the disclosure have a structure of formula (X″), wherein R⁶ is C₁₋₆ alkyl.

In some embodiments, compounds of the disclosure have a structure of formula (X″), wherein R⁵ and R⁶ are taken together with the carbon to which they are attached to form 3-4 membered cycloalkyl ring.

In some embodiments, compounds of the disclosure have a structure of formula (X″), wherein R⁵ and R⁶ may be taken together with the carbon to which they are attached to form 3-4 membered heterocycle ring.

In certain aspects the present invention provides a compound of Formula I:

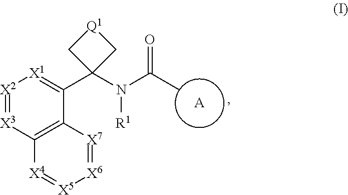

or a pharmaceutically acceptable salt, isotopic derivative or prodrug thereof, or a pharmaceutical composition thereof;

In other aspects the present invention provides a compound of Formula VIII, Formula IX, Formula X, Formula XI, or Formula XIII:

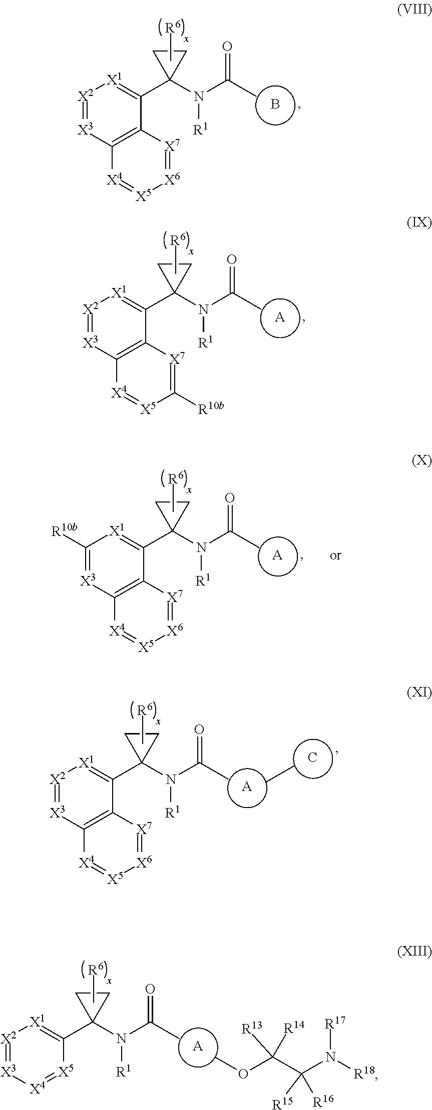

or a pharmaceutically acceptable salt, isotopic derivative prodrug, or pharmaceutical composition thereof;

In other aspects the present invention provides a compound of Formula XIV, Formula XV, Formula XVI, Formula XVII, Formula XIX, Formula XX, Formula XXI, Formula XXI, Formula XXIII, Formula XXIV, Formula XXV, Formula XXVII, Formula XXVIII, or Formula XXIX:

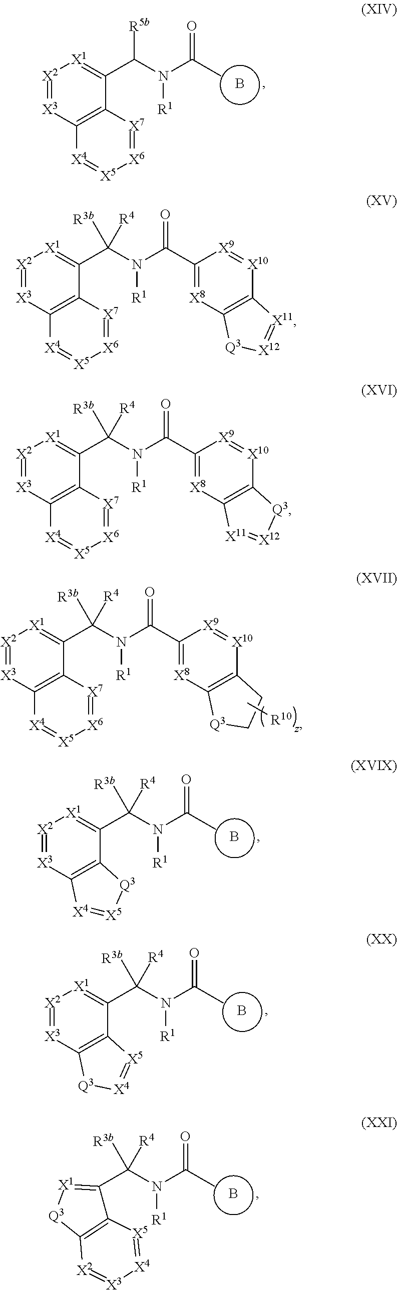

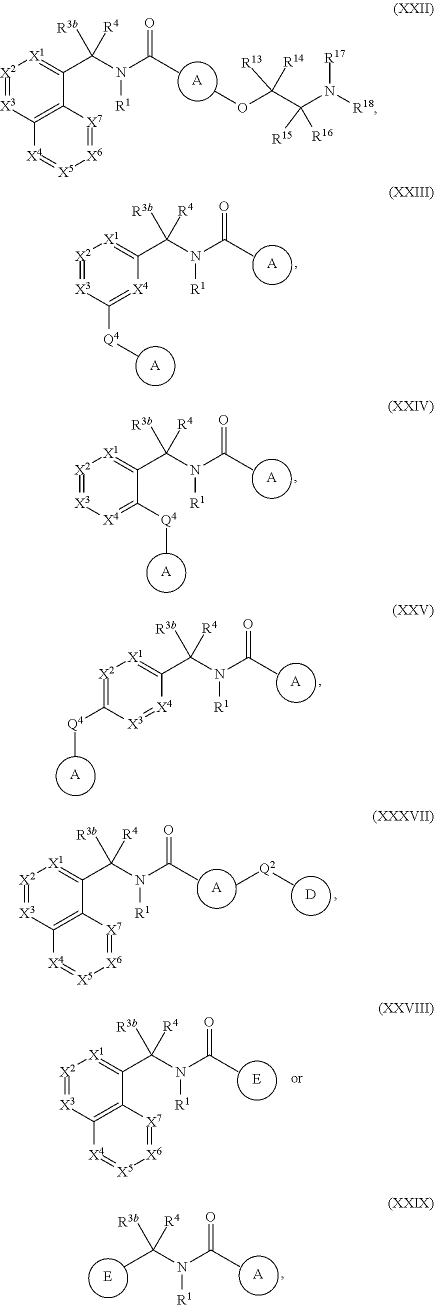

or a pharmaceutically acceptable salt, isotopic derivative, prodrug, or pharmaceutical composition thereof;
wherein:
Q¹ is O or NR²;
Q² is O, NR², or CR³R⁴;
Q³ is S, O, or NR²;
Q⁴ is bond, O, NR², or CR³R⁴;
z is 0, 1, 2, or 3;
x is 0, 1, 2, 3, or 4;
each

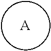

is an aryl, heteroaryl, heterocycle, cycloalkene, or bicycle optionally substituted with 1, 2, 3, or 4 substituents independently selected from R¹⁰ and optionally substituted with 1 R¹¹ substituent;

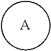

is an aryl, heteroaryl, heterocycle, or bicycle optionally substituted with 1, 2, 3, or 4 substituents independently selected from R¹⁰ and substituted with 1 R¹¹ substituent;

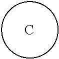

is an aryl, heteroaryl, heterocycle, or bicycle optionally substituted with 1, 2, 3, or 4 substituents independently selected from R²⁰;

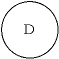

is a cycloalkyl, aryl, heteroaryl, heterocycle, or bicycle optionally substituted with 1, 2, 3, or 4 substituents independently selected from R²⁰ and optionally substituted with 1 R¹¹ substituent;

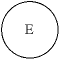

is a tricycle optionally substituted with 1, 2, 3, or 4 substituents independently selected from R²⁰;
X¹, X², X³, X⁴, X⁵, X⁶, and X⁷ are independently selected from the group consisting of CR¹⁰, CH, and N, wherein no more than four of X¹, X², X³, X⁴, X⁵, X⁶, and X⁷ are N;
X⁸, X⁹, X¹⁰, X¹¹, and X¹² are independently selected from the group consisting of CH, CR¹⁰, CR¹¹, and N, wherein no more than four of X¹, X², X³, X⁴, X⁵, X⁶, and X⁷ are N;
each R¹ is independently hydrogen, alkyl, haloalkyl, alkenyl, alkynyl, aryl, heteroaryl, or cycloalkyl;
each R² is independently hydrogen, alkyl, haloalkyl, alkenyl, alkynyl, aryl, heteroaryl, cycloalkyl, alkyl-heterocycle, alkyl-heteroaryl, alkyl-aryl, or —C(O)R⁷;
R³ and R⁴ are independently selected from the group consisting of hydrogen, halogen, alkyl, haloalkyl, alkenyl, alkynyl, aryl, heteroaryl, and cycloalkyl;
R^{3b} is alkyl, haloalkyl, alkenyl, alkynyl, aryl, heteroaryl, and cycloalkyl;
or R³ and R⁴ or R^{3b} and R⁴ are combined with the carbon to which they are attached to form a spirocyclopropyl, wherein the spirocyclopropyl group is optionally substituted with 1, 2, 3, or 4 substituents independently selected from R⁶;
R⁵ is alkenyl or alkynyl;
R^{5b} is haloalkyl;
each R⁶ is independently selected from the group consisting of halogen, alkyl, haloalkyl, alkenyl, alkynyl, aryl, heteroaryl, and cycloalkyl;
each R⁷ and R⁸ is independently selected from the group consisting of hydrogen, alkyl, haloalkyl, alkenyl, alkynyl, aryl, heteroaryl, cycloalkyl, —NR¹R¹², —OR¹², and SR¹²;
each R¹⁰ is independently selected from the group consisting of hydrogen, halogen, alkyl, haloalkyl, cyano, nitro, alkenyl, alkynyl, aryl, heteroaryl, cycloalkyl, -alkyl-NR¹R², -alkyl-OR²,
   —C(O)R⁷, —NR²C(O)R⁷, —OC(O)R⁷, —NR¹R², —OR², SR², —S(O)R⁷, S(O)₂R⁷, and —P(O)R⁷R⁸, each of which except hydrogen, cyano, and nitro is optionally substituted with 1, 2, 3, or 4 substituents independently selected from R²¹;
R^{10b} is selected from the group consisting of halogen, alkyl, haloalkyl, cyano, nitro, alkenyl, alkynyl, aryl, heteroaryl, cycloalkyl, -alkyl-NR¹R², -alkyl-OR², —C(O)R⁷, —NR²C(O)R⁷, —OC(O)R⁷, —NR¹R², —OR², SR², —S(O)R⁷, S(O)₂R⁷, and —P(O)R⁷R⁸, each of which except hydrogen, cyano, and nitro is optionally substituted with 1, 2, 3, or 4 substituents independently selected from R²¹;

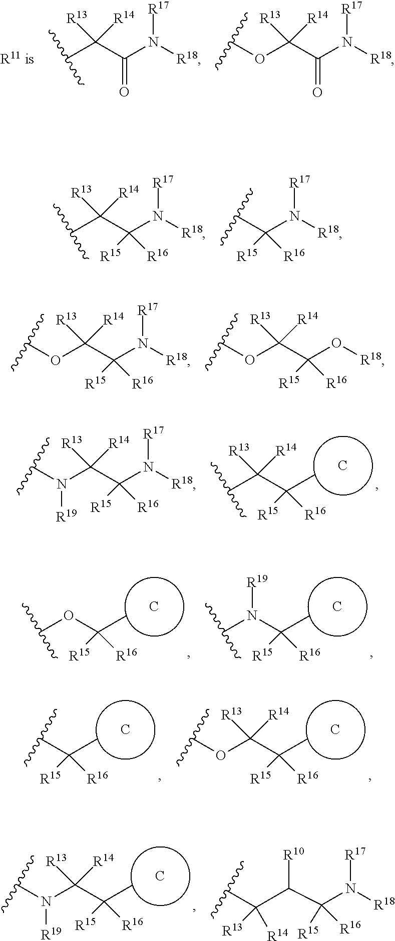

### or CH²CN;

each R¹² is independently selected from hydrogen, alkyl, haloalkyl, alkenyl, alkynyl, aryl, heteroaryl, and cycloalkyl;
R¹³, R¹⁴, R¹⁵, and R¹⁶ are independently selected from hydrogen, alkyl, haloalkyl, alkenyl, alkynyl, aryl, heteroaryl, cycloalkyl, CH₂OR², and CH₂NR¹R²;
or R¹³ and R¹⁴ are combined with the carbon to which they are attached to form a spirocyclopropyl;
or R¹⁵ and R¹⁶ are combined with the carbon to which they are attached to form a spirocyclopropyl;
R^{16b} is alkyl, haloalkyl, alkenyl, alkynyl, aryl, heteroaryl, or cycloalkyl;
R¹⁷ is hydrogen, alkyl, haloalkyl, alkenyl, alkynyl, aryl, heteroaryl, -alkylcycloalkyl, —CH₂CH₂—OR², —CH₂CH₂—NR¹R², -alkyl-aryl, -alkyl-heteroaryl, or cycloalkyl;
R¹⁸ is hydrogen, alkyl, haloalkyl, alkenyl, alkynyl, aryl, heteroaryl, cycloalkyl, or —C(O)R⁷;
or R¹⁷ and R¹⁸ together with the nitrogen to which they are attached form a heterocycle;
R¹⁹ is hydrogen, alkyl, haloalkyl, alkenyl, alkynyl, aryl, heteroaryl, cycloalkyl, or —C(O)R⁷;
each R²⁰ is independently selected from hydrogen, halogen, alkyl, haloalkyl, cyano, nitro, oxo, alkenyl, alkynyl, aryl, heteroaryl, cycloalkyl, -alkyl-NR¹R², -alkyl-OR², —C(O)R⁷, —NR²C(O)R⁷,
   —OC(O)R⁷, —NR¹R², —OR², SR², —S(O)R⁷, S(O)₂R⁷, and —P(O)R⁷R⁸, each of which except hydrogen, cyano, and nitro is optionally substituted with 1, 2, 3, or 4 substituents independently selected from R²²;
each R²¹ is independently selected from hydrogen, halogen, alkyl, haloalkyl, cyano, nitro, alkenyl, alkynyl, aryl, heteroaryl, cycloalkyl, —C(O)R⁷, —NR²C(O)R⁷, —OC(O)R⁷, —NR¹R², —OR², SR², —S(O)R⁷, S(O)₂R⁷, and —P(O)R⁷R⁸; and
each R²² is independently selected from hydrogen, halogen, alkyl, haloalkyl, cyano, nitro, alkenyl, alkynyl, aryl, heteroaryl, cycloalkyl, —C(O)R⁷, —NR²C(O)R⁷, —OC(O)R⁷, —NR¹R², —OR², SR², —S(O)R⁷, S(O)₂R⁷, and —P(O)R⁷R⁸.

The present invention provides a compound of Formula (Z):

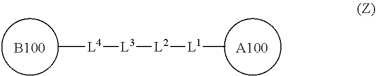

or a pharmaceutically acceptable salt, prodrug, or pharmaceutical composition thereof.

In the compound of Formula (Z), A100 may be selected from the group consisting of Formulas (A1), (A2), (A3), (A4), (A5), (A6), (A7), (A8), (A9), (A10), (A11), (A12), (A13), (A14), (A15), (A16), (A17), (A18), (A19), and (A20):

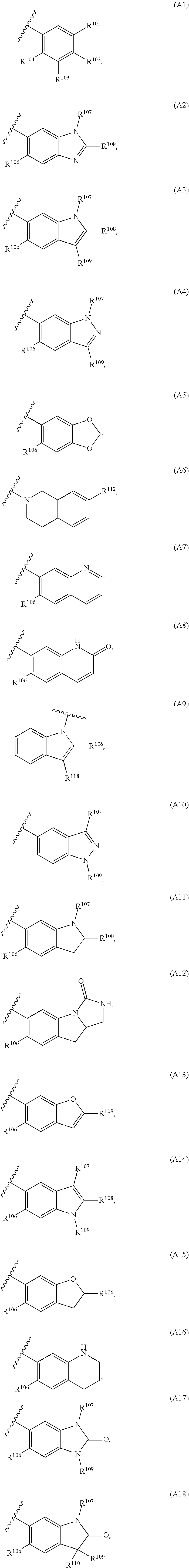

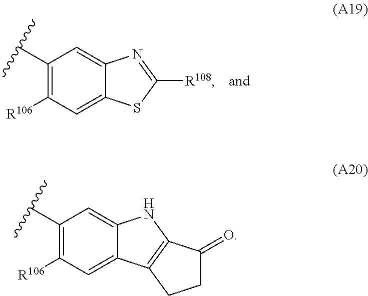

In the compound of Formula (Z), L¹ may be —CH₂—, or may be absent.

In the compound of Formula (Z), L² may be —CONH—, —NHCO—, —NHSO₂—, —SO₂NH—, —NH—, —CO—, —NR¹¹⁴- or may be absent.

In the compound of Formula (Z), L³ may be:

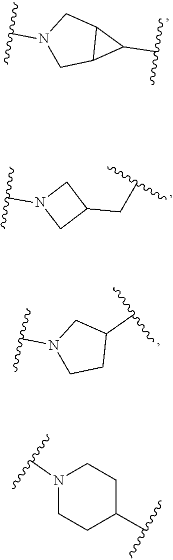 [SMILES: *C1C2CN(*)CC12 ; *CC1CN(*)C1 ; *C1CCN(*)C1 ; *C1CCN(*)CC1]

or may be absent.

In the compound of Formula (Z), L⁴ may be —CR₁₂₀R₁₂₁— or may be absent.

In the compound of Formula (Z), B100 may be:

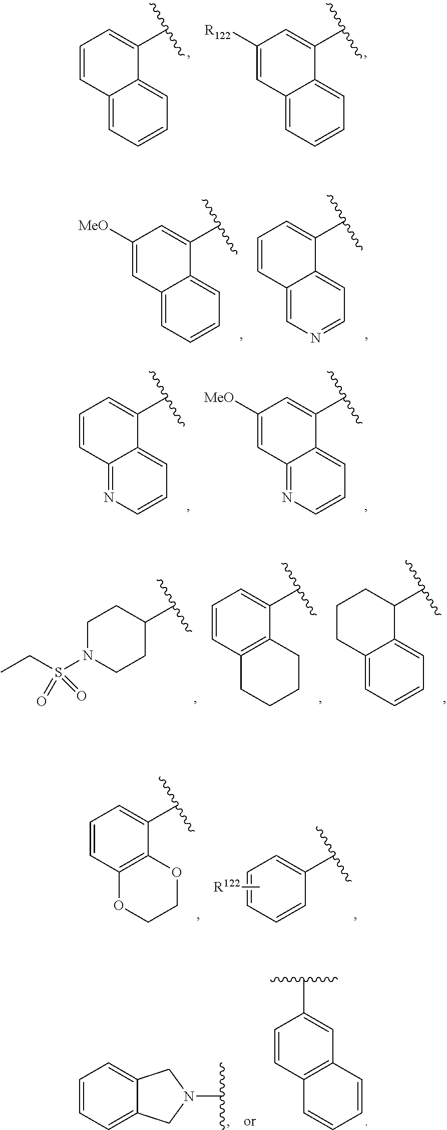

In the compound of Formula (Z), R¹⁰¹ may be H, halo, —OH, —CONH₂, —OR¹²⁴, NR¹²⁴R¹²⁵, —NO₂, —NS(O)₂CH³, C₁-C₆ alkoxyl, C₃-C₆ cycloalkoxyl, C₃-C₆ cycloheteroalkoxyl, C₁-C₆ heteroalkoxyl, —C(O)CH₃,

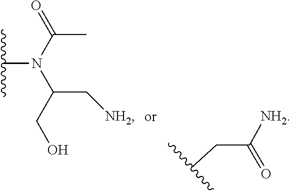

R¹⁰² may be H, —OH, halo, or an alkyne, including substituted alkynes, for example

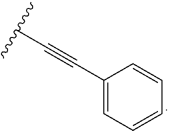 [SMILES: *C#Cc1ccccc1]

R¹⁰³ may be H or halo, or alkyl.

R¹⁰⁴ may be H, halo, or alkyl.

R¹⁰⁶ may be H, halo or C₁-C₆ alkyl, or a C₁-C₆ cycloheteroalkyl.

R¹⁰⁷ may be H, Me, —COH, —CH₂OH, halo, C₁-C₃ alkyl, C₁-C₃ heteroalkyl, or C₁-C₄ cycloheteroalkyl, for example:

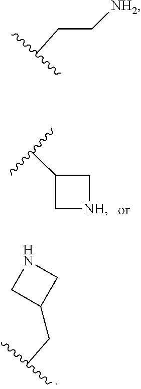

R¹⁰⁸ may be H, halo, CO₂H, CONH₂, aryl, C₃-C₆ heteroaryl (for example pyridine), C₁-C₆ alkyl, C₁-C₆ haloalkyl, C₃-C₆ cycloalkyl, C₁-C₆ heteroalkyl, C₃-C₆ cycloheteroalkyl (for example morpholine), or for example

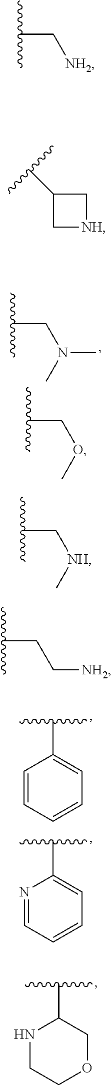

or may be

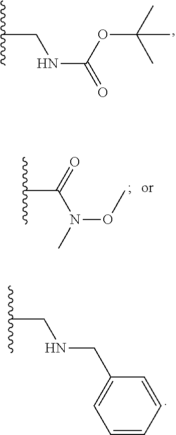

R¹⁰⁹ may be H, halo, or Me.

R¹¹⁰ may be H or Me.

R¹¹² may be NO₂ or NH₂.

R¹¹⁴ may be Me.

R¹¹⁸ may be

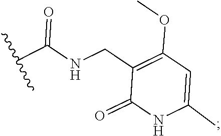 [SMILES: *C(=O)NCc1c(OC)cc(C)[nH]c1=O]

R¹²⁰ may be H, Me, halo, cyano, C₁-C₆ alkyl, C₁-C₆ haloalkyl, C₃-C₆ cycloalkyl, C₁-C₆ heteroalkyl, C₃-C₆ cycloheteroalkyl, aryl, or heteroaryl.

R¹²¹ may be H.

In alternative aspects of the invention, R¹²⁰ and R¹²¹ may together with the carbon atoms to which they are attached form a C₃-C₆ cycloalkyl or a C₃-C₆ cycloheteroalkyl.

R¹²² may be H, Me, halo, aryl, C₁-C₆ alkyl, C₃-C₆ cycloalkyl, C₃-C₆ cycloheteroalkyl, for example a phenyl, benzyl, or thiophene or,

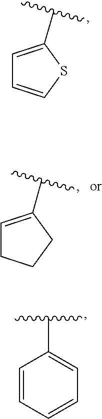 [SMILES: *c1cccs1 ; *C1=CCCC1 ; *c1ccccc1]

or may be

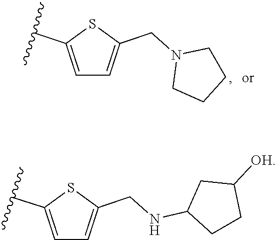 [SMILES: *c1ccc(CN2CCCC2)s1 ; *c1ccc(CNC2CCC(O)C2)s1]

R¹²² may be OR¹²³.

R¹²³ may be aryl, C₃-C₆ cycloalkyl, or C₃-C₆ cycloheteroalkyl (for example, phenyl or benzyl).

R¹²⁴ may be H, —S(O)₂CH₃, C₁-C₆ alkyl, C₃-C₆ cycloalkyl, C₁-C₆ heteroalkyl, C₃-C₆ cycloheteroalkyl, or heteroaryl, or

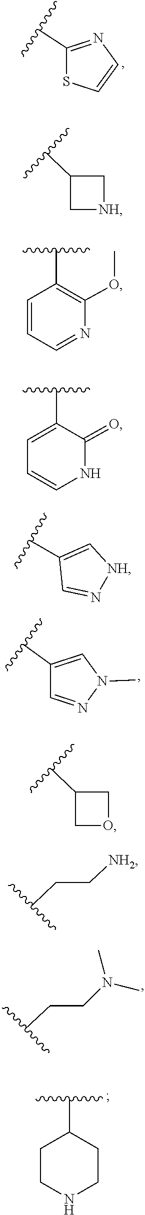

or may be

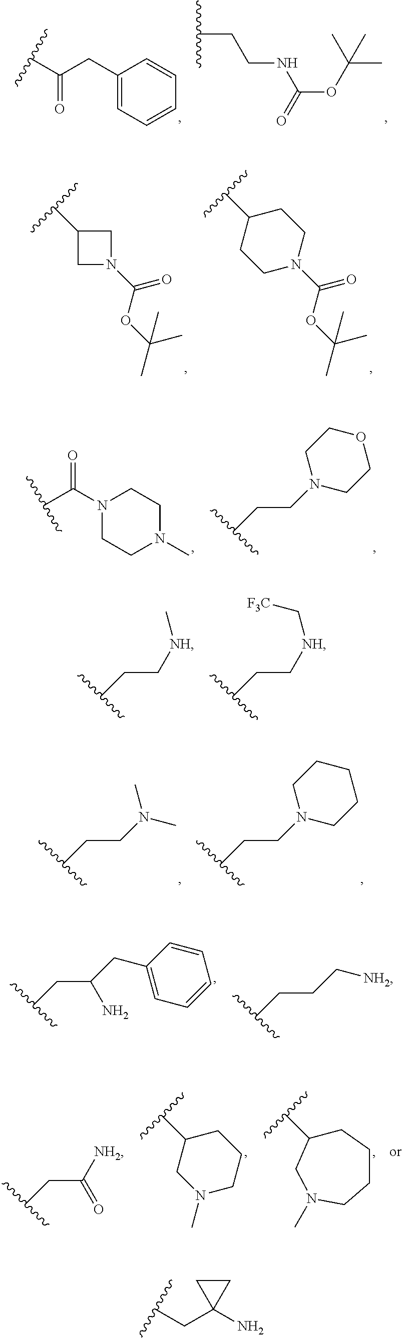

R¹²⁵ may be H, C(O)CH₃, or may be absent.

In the compound of Formula (Z), each alkoxyl, alkyl, aryl, cycloalkoxyl, cycloalkyl, cycloheteroalkoxyl, cycloheteroalkyl, haloalkyl, heteroalkoxyl, heteroalkyl, and heteroaryl group may be optionally substituted.

Aspects of the invention include salt forms of the compound of Formula (Z). Salt forms may comprise any pharmaceutically acceptable salt, for example hydrochloric acid (HCl), trifluoroacetic acid (TFA), or formic acid (FA).

In preferred embodiments, R¹⁰⁶ may be —CH₃.

L² may be —NHCO—.

L⁴ may be —CR¹²⁰R¹²¹—. R¹²⁰ may be CH₃ and R¹²¹ may be H.

In alternative aspects, L⁴ may be —CR¹²⁰R¹²¹, and R¹²⁰ and R¹²¹ together with the carbon atoms to which they are attached form a (hetero)cycloalkyl of formula (Y1), (Y2), or (Y3):

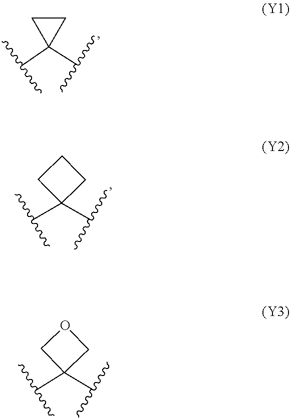 [SMILES: *C1(*)CC1 ; *C1(*)CCC1 ; *C1(*)COC1]

B100 may be

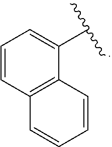 [SMILES: *c1cccc2ccccc12]

In further alternative aspects, L⁴ may be —CR¹²⁰R¹²¹, wherein R¹²⁰ may be CH₂F, CHF, CF³, or may be selected from the group consisting may be of the formula (X6):

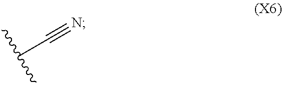 [SMILES: *C#N]

### and R¹²¹ may be H.

In certain embodiments a compound of the present invention is of Formula:

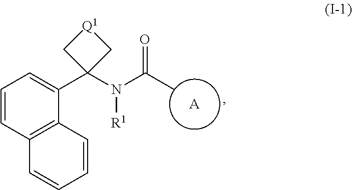

or a pharmaceutically acceptable salt thereof.

In certain embodiments a compound of the present invention is of Formula:

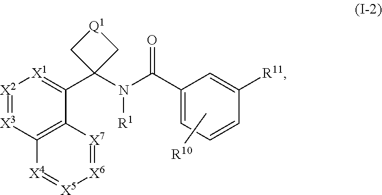

or a pharmaceutically acceptable salt thereof;
In certain embodiments a compound of the present invention is of Formula:

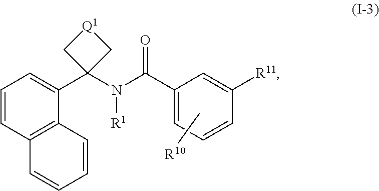

or a pharmaceutically acceptable salt thereof;

In certain embodiments a compound of the present invention is of Formula:

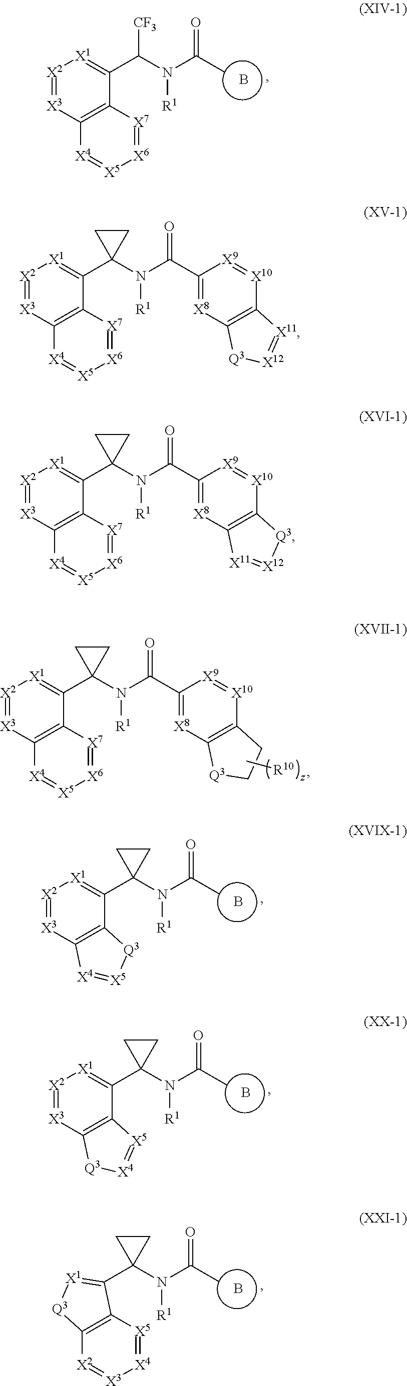

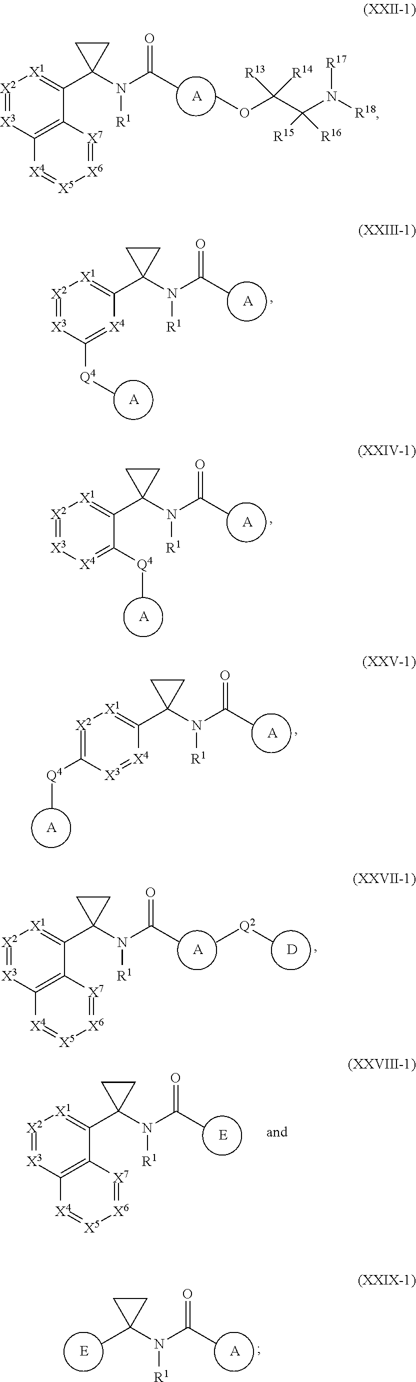

or a pharmaceutically acceptable salt thereof.

In certain embodiments a compound of the present invention is of Formula:

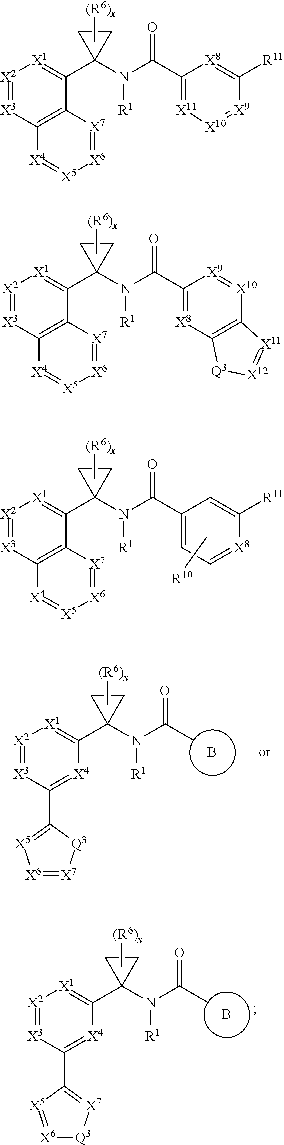

or a pharmaceutically acceptable salt thereof.

Aspects of the invention provide a composition comprising the compounds according to the invention.

Aspects of the invention provide a pharmaceutically acceptable salt or a prodrug of the compounds of the invention.

Aspects of the invention provide a method of treating a subject afflicted with a viral infection, the method comprising administering to the subject a compound according to the invention.

Aspects of the invention provide a method of preventing a viral infection in a subject, the method comprising administering to the subject a compound according to the invention.

Aspects of the invention provide a method of preventing viral replication in a subject, the method comprising administering to the subject a compound according to the invention.

Aspects of the invention provide a method of inhibiting PLpro in the subject, the method comprising administering to the subject a compound according to the invention

### Embodiments of the Present Invention

In certain embodiments a compound of the present invention is of Formula:

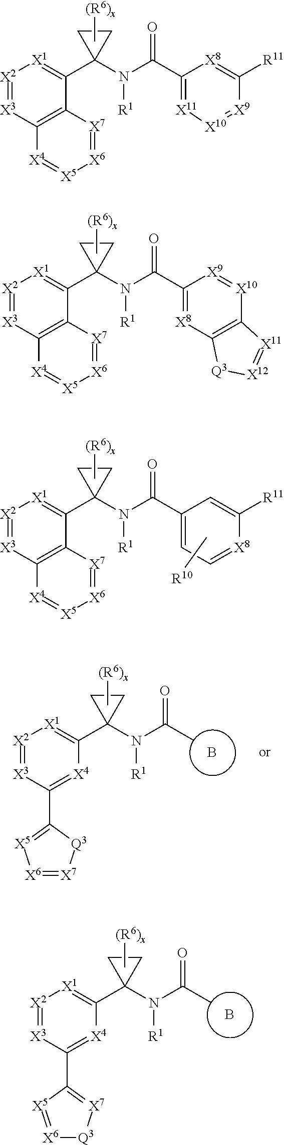

or a pharmaceutically acceptable salt thereof, wherein X¹, X², X³, X⁴, X⁵, X⁶, X⁷, X⁸, x⁹, X¹⁰, X¹¹, X¹², R¹, R⁶, R¹⁰, R¹¹, Q³, Ring B, and x are as defined herein.

### Embodiments of

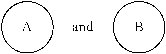

In certain embodiments,

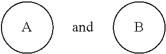

are selected from:

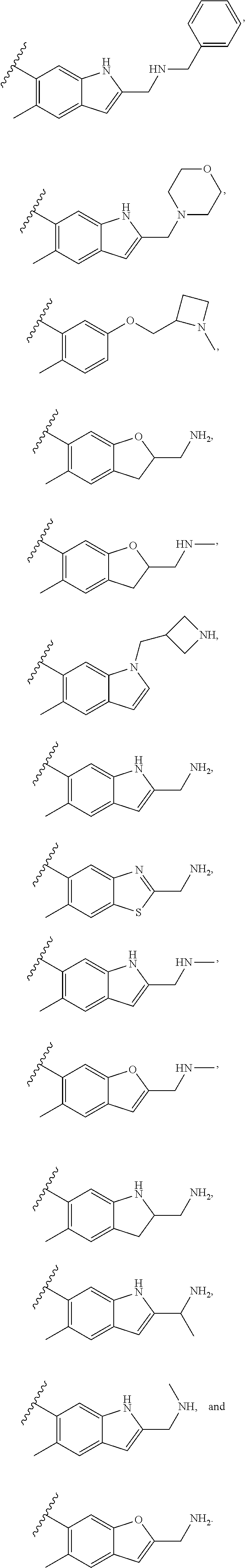

In certain embodiments,

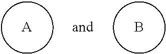

are selected from:

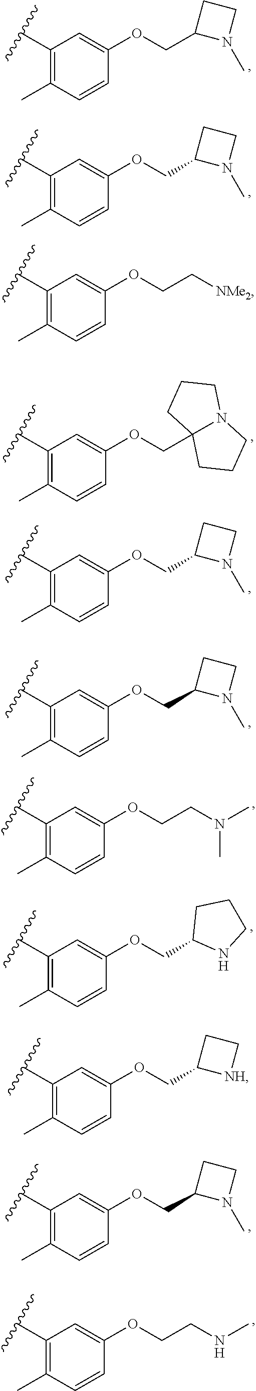

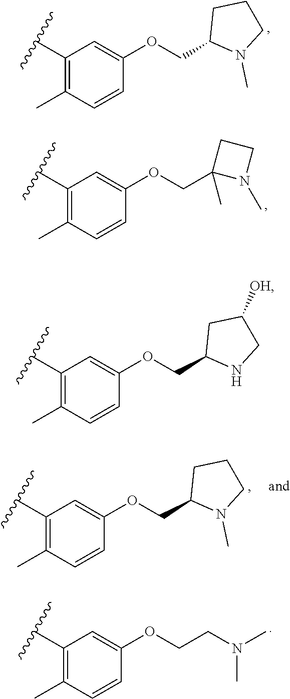 [SMILES: *c1cc(OC[C@@H]2CCCN2C)ccc1C ; *c1cc(OCC2(C)CCN2C)ccc1C ; *c1cc(OC[C@H]2C[C@H](O)CN2)ccc1C ; *c1cc(OC[C@H]2CCCN2C)ccc1C ; *c1cc(OCCN(C)C)ccc1C]

In certain embodiments,

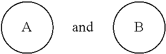

are selected from:

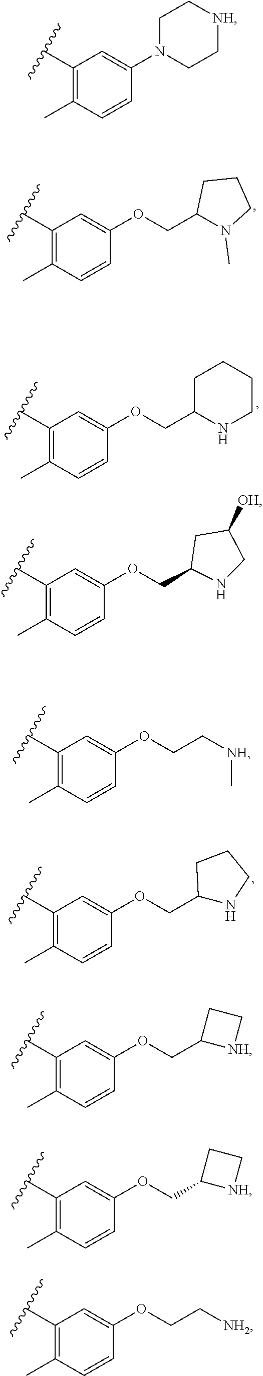

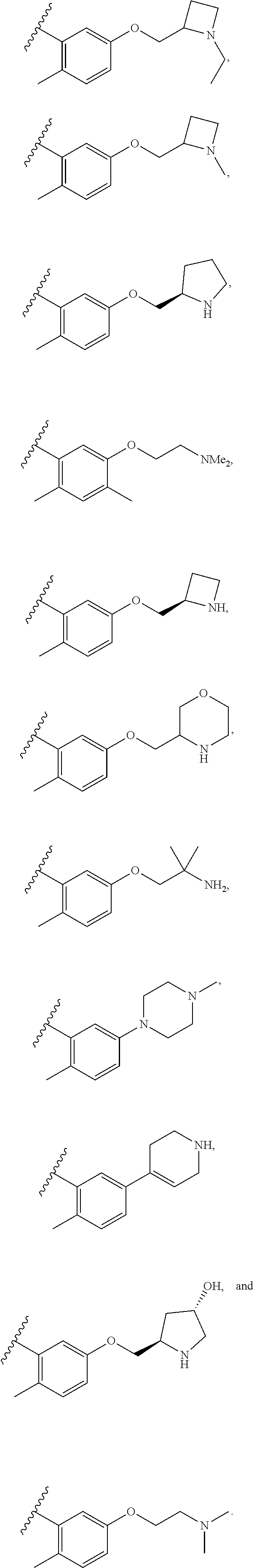

In certain embodiments,

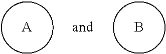

are selected from:

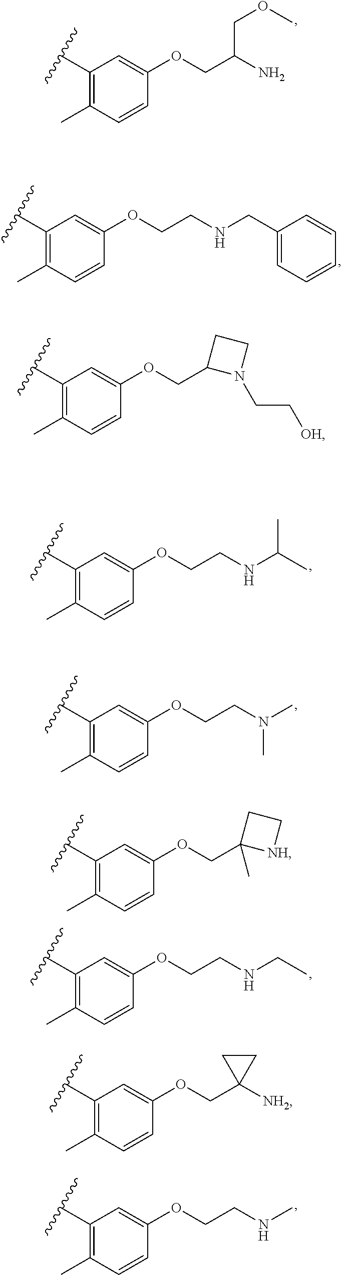

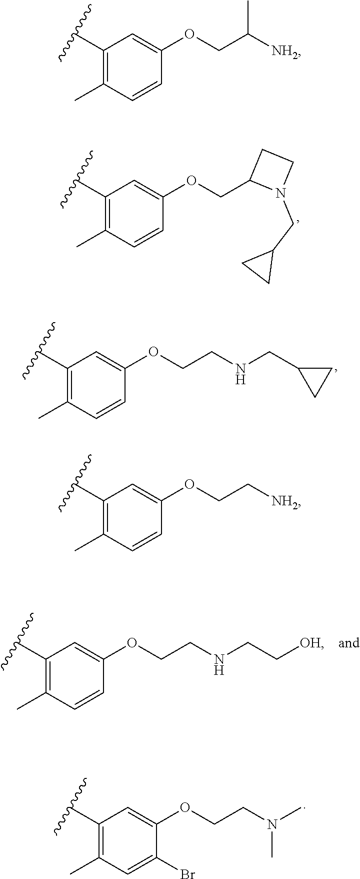 [SMILES: *c1cc(OCC(C)N)ccc1C ; *c1cc(OCC2CCN2CC2CC2)ccc1C ; *c1cc(OCCNCC2CC2)ccc1C ; *c1cc(OCCN)ccc1C ; *c1cc(OCCNCCO)ccc1C ; *c1cc(OCCN(C)C)c(Br)cc1C]

In certain embodiments,

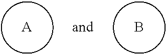

are selected from:

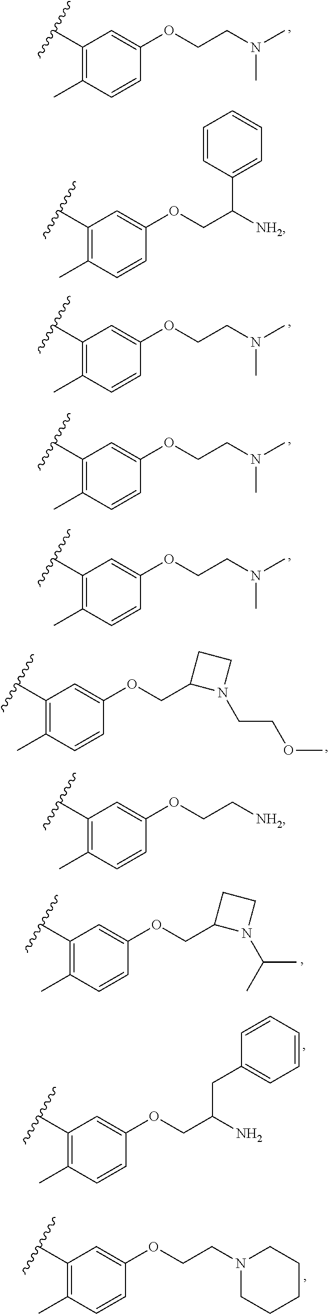

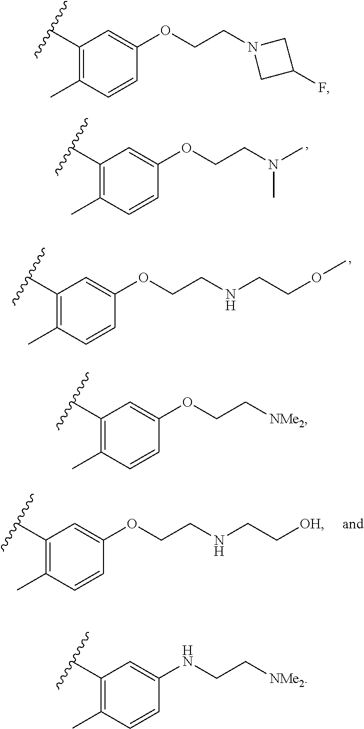

In certain embodiments,

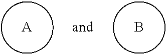

are selected from:

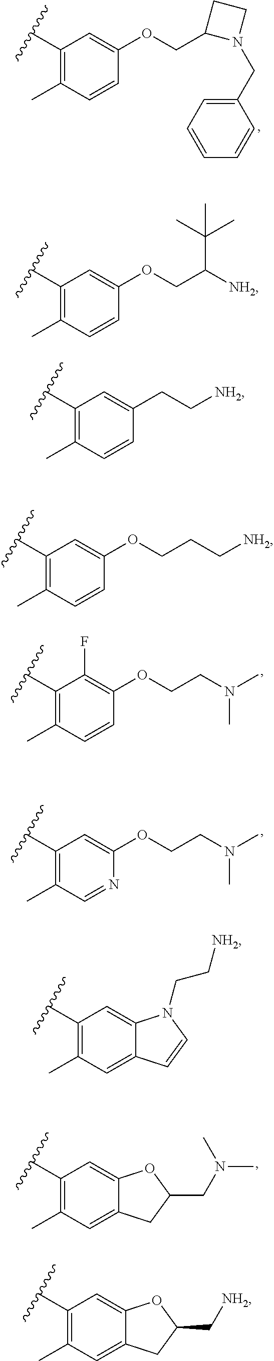

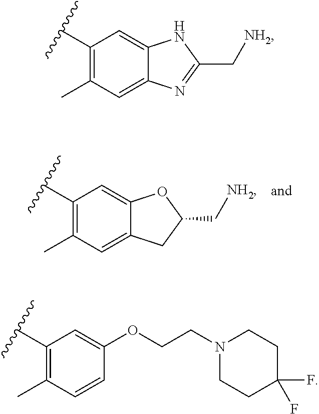 [SMILES: *c1cc2[nH]c(CN)nc2cc1C ; *c1cc2c(cc1C)C[C@@H](CN)O2 ; *c1cc(OCCN2CCC(F)(F)CC2)ccc1C]

In certain embodiments,

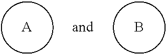

are selected from:

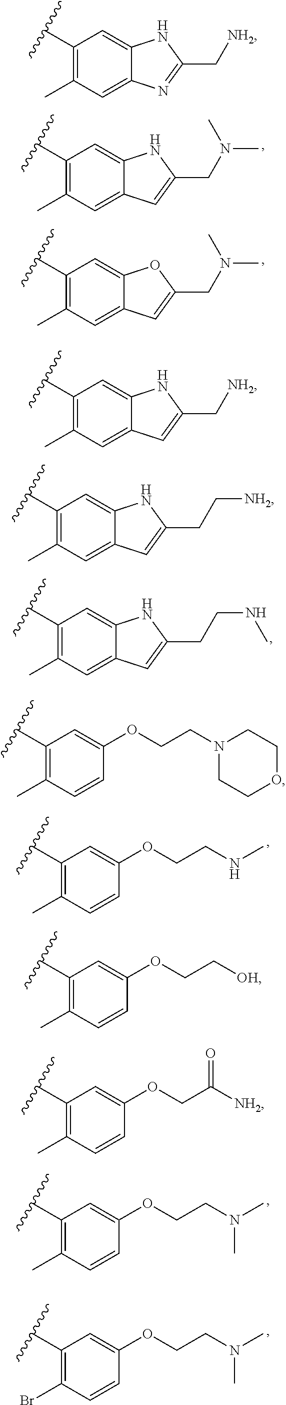

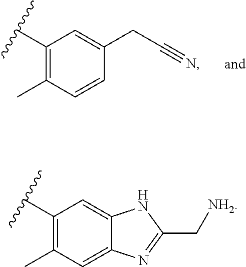 [SMILES: *c1cc(CC#N)ccc1C ; "and" ; *c1cc2[nH]c(CN)nc2cc1C]

In certain embodiments,

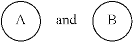

are selected from:

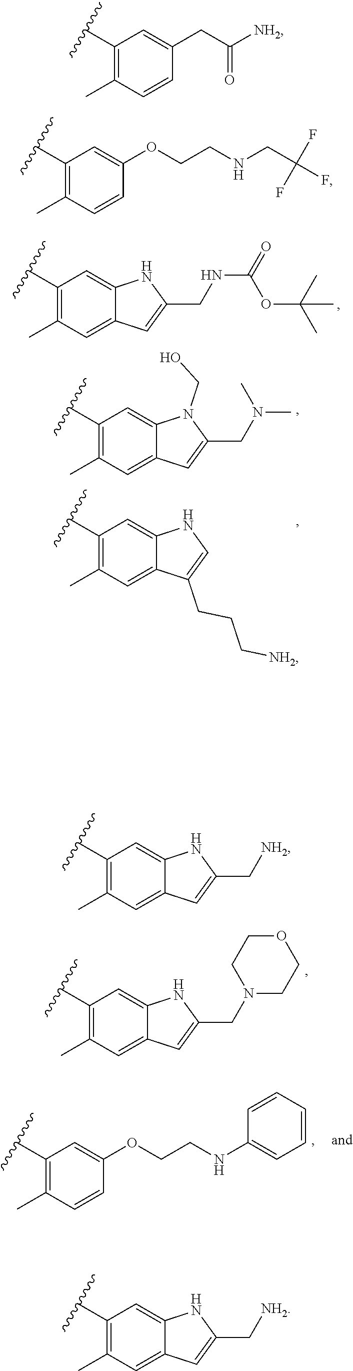

In certain embodiments,

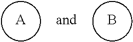

are selected from:

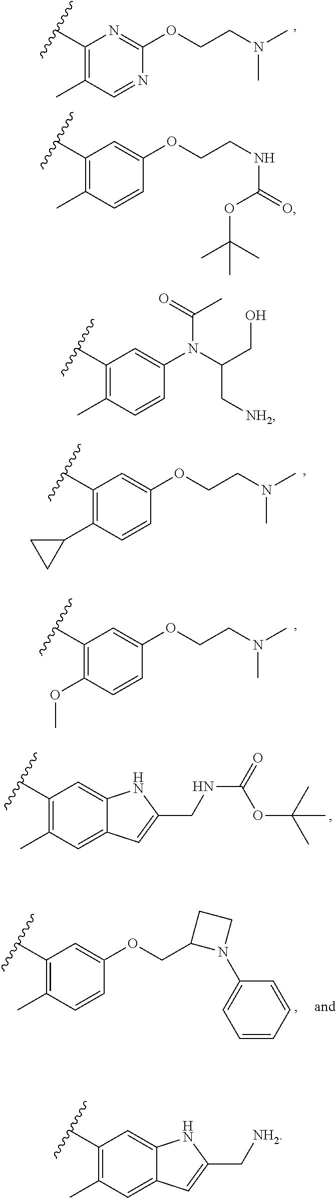

In certain embodiments,

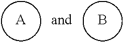

are selected from:

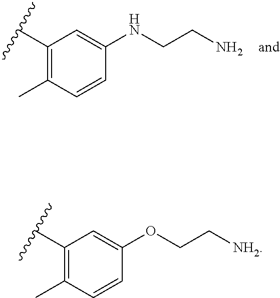 [SMILES: *c1cc(NCCN)ccc1C ; *c1cc(OCCN)ccc1C]

In certain embodiments,

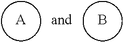

are:

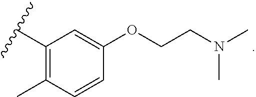

In certain embodiments,

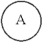

is selected from:

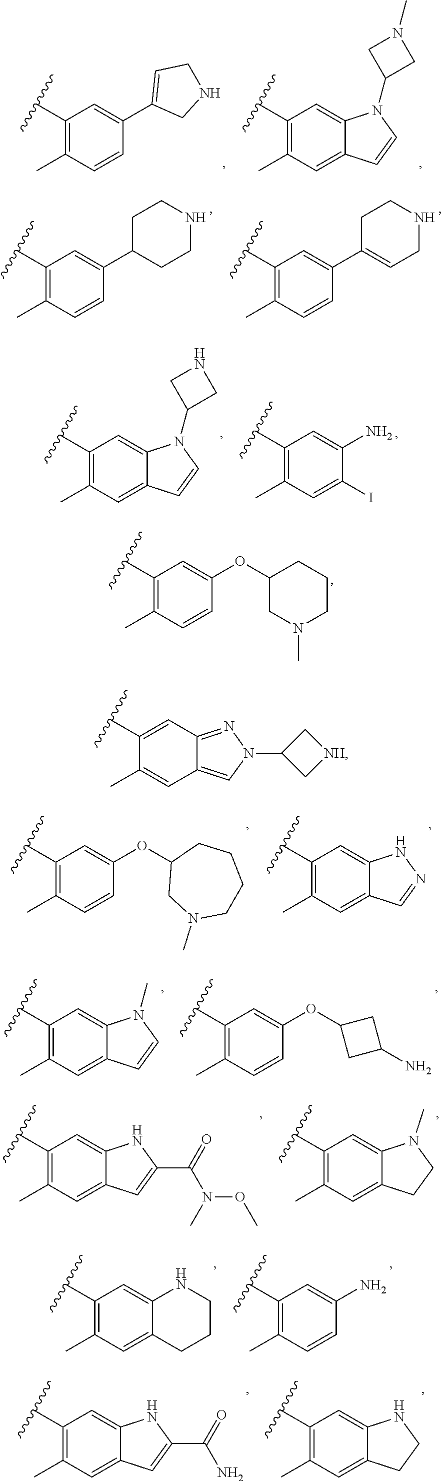

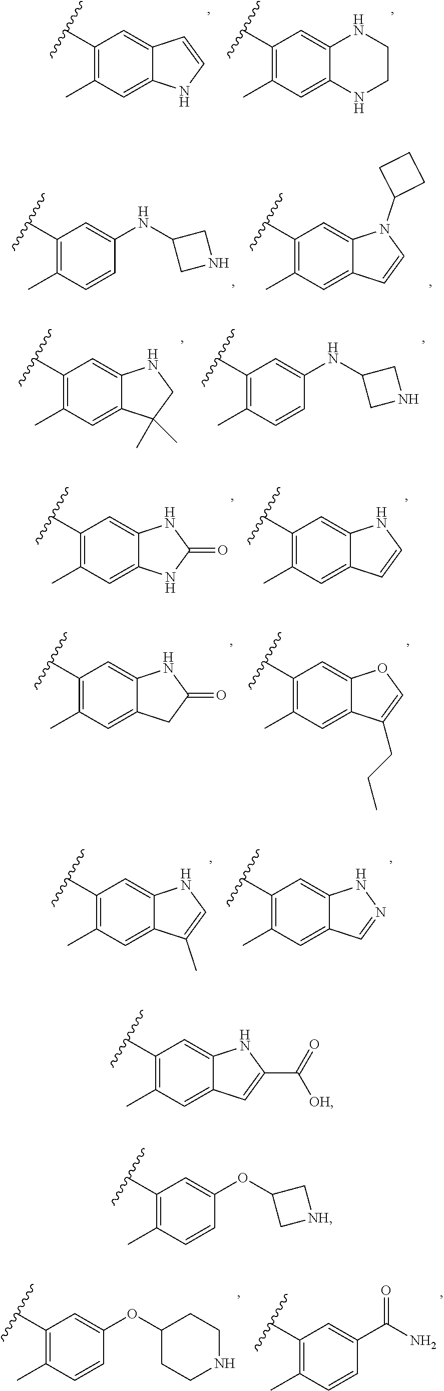

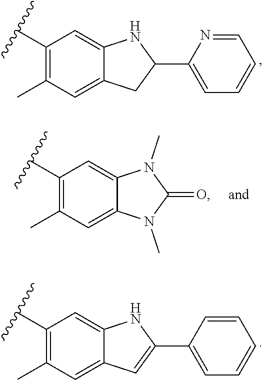 [SMILES: *c1cc2c(cc1C)CC(c1ccccn1)N2 ; *c1cc2c(cc1C)n(C)c(=O)n2C ; *c1cc2[nH]c(-c3ccccc3)cc2cc1C]

In certain embodiments,

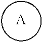

is selected from:

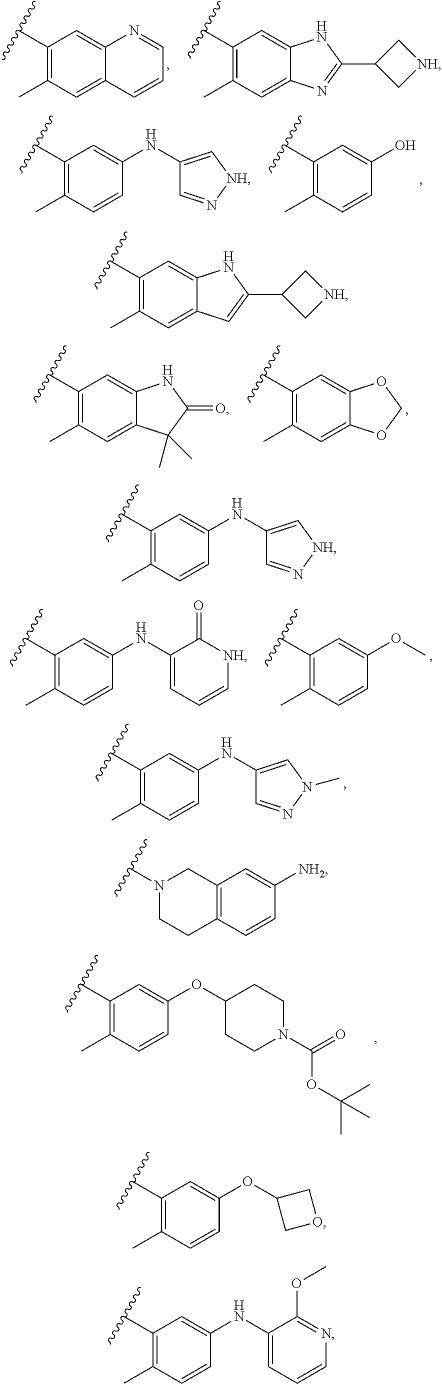

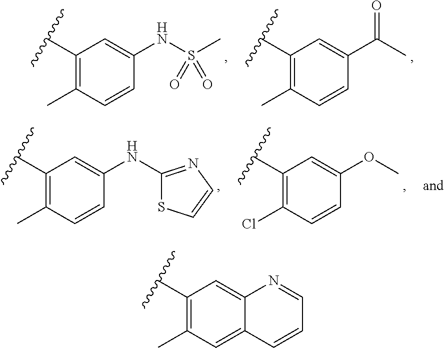

In certain embodiments,

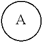

is selected from:

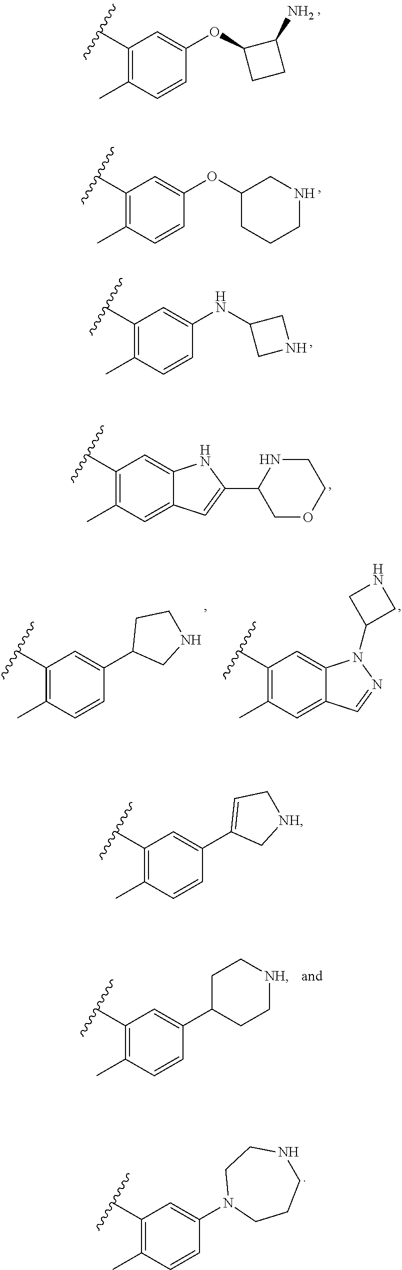

### Embodiments of

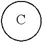

In certain embodiments,

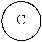

is selected from:

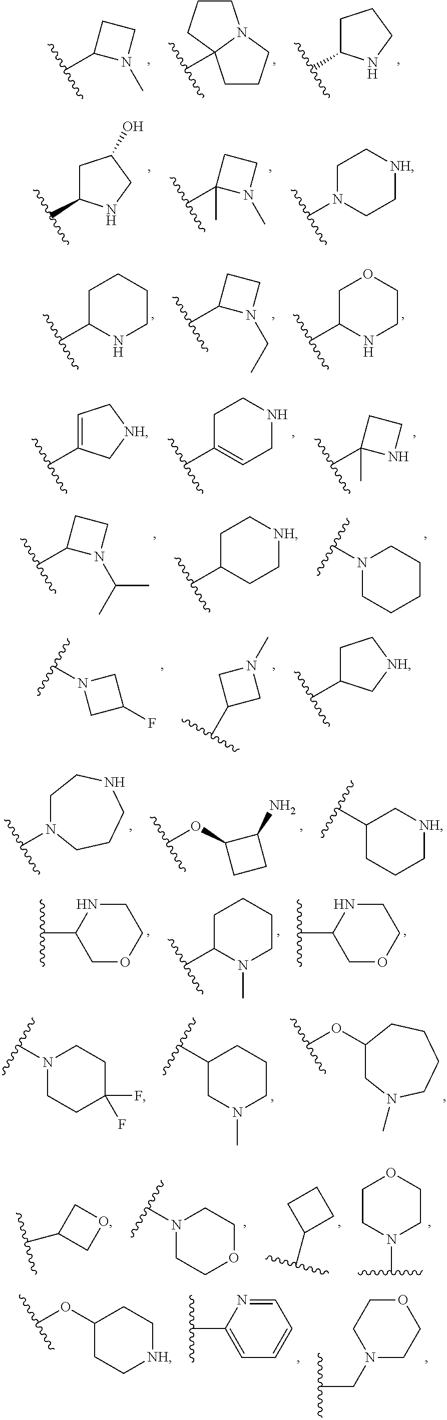

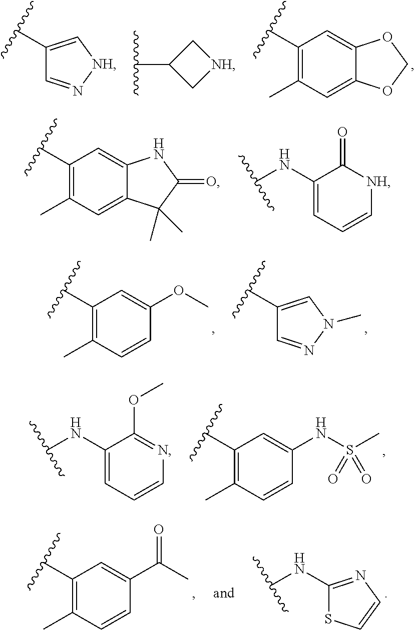

### Non-Limiting Embodiments of R¹

In certain embodiments, R¹ is hydrogen.

In certain embodiments, R¹ is alkyl.

In certain embodiments, R¹ is haloalkyl.

In certain embodiments, R¹ is alkenyl.

In certain embodiments, R¹ is alkynyl.

In certain embodiments, R¹ is aryl.

In certain embodiments, R¹ is heteroaryl.

In certain embodiments, R¹ is hydrogen.

In certain embodiments, R¹ is cycloalkyl.

### Non-Limiting Embodiments of R²

In certain embodiments, R² is hydrogen.

In certain embodiments, R² is alkyl.

In certain embodiments, R² is haloalkyl.

In certain embodiments, R² is alkenyl.

In certain embodiments, R² is alkynyl.

In certain embodiments, R² is aryl.

In certain embodiments, R² is heteroaryl.

In certain embodiments, R² is cycloalkyl.

In certain embodiments, R² is —C(O)R⁷.

### Non-Limiting Embodiments of R³

In certain embodiments, R³ is hydrogen

In certain embodiments, R³ is halogen

In certain embodiments, R³ is alkyl.

In certain embodiments, R³ is haloalkyl.

In certain embodiments, R³ is alkenyl.

In certain embodiments, R³ is alkynyl.

In certain embodiments, R³ is aryl.

In certain embodiments, R³ is heteroaryl.

In certain embodiments, R³ is cycloalkyl.

### Non-Limiting Embodiments of R^{3b}

In certain embodiments, R^{3b} is alkyl.

In certain embodiments, R^{3b} is haloalkyl.

In certain embodiments, R^{3b} is alkenyl.

In certain embodiments, R^{3b} is alkynyl.

In certain embodiments, R^{3b} is aryl.

In certain embodiments, R^{3b} is heteroaryl.

In certain embodiments, R^{3b} is cycloalkyl.

### Non-Limiting Embodiments of R⁴

In certain embodiments, R⁴ is hydrogen.

In certain embodiments, R⁴ is halogen.

In certain embodiments, R⁴ is alkyl.

In certain embodiments, R⁴ is haloalkyl.

In certain embodiments, R⁴ is alkenyl.

In certain embodiments, R⁴ is alkynyl.

In certain embodiments, R⁴ is aryl.

In certain embodiments, R⁴ is heteroaryl.

In certain embodiments, R⁴ is cycloalkyl.

### Non-Limiting Embodiments of R⁵

In certain embodiments, R⁵ is alkenyl.

In certain embodiments, R⁵ is alkynyl.

In certain embodiments, R⁵ is

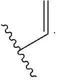 [SMILES: *C=C]

In certain embodiments, R⁵ is

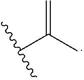 [SMILES: *C(=C)C]

In certain embodiments, R⁵ is

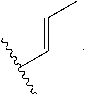 [SMILES: */C=C/C]

In certain embodiments, R⁵ is

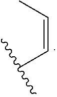 [SMILES: */C=C\C]

In certain embodiments, R⁵ is

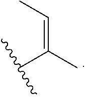 [SMILES: */C(C)=C\C]

In certain embodiments, R⁵ is

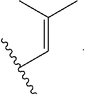 [SMILES: *C=C(C)C]

In certain embodiments, R⁵ is

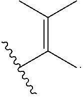 [SMILES: *C(C)=C(C)C]

In certain embodiments, R⁵ is

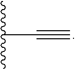 [SMILES: *C#C]

In certain embodiments, R⁵ is

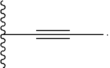 [SMILES: *C#CC]

### Non-Limiting Embodiments of R^{5b}

In certain embodiments, R^{5b} is haloalkyl.

In certain embodiments, R^{5b} is

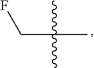 [SMILES: *CF]

In certain embodiments, R^{5b} is

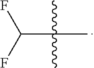 [SMILES: *C(F)F]

In certain embodiments, R^{5b} is —CF₃.

### Non-Limiting Embodiments of R⁶

In certain embodiments, R⁶ is halogen.

In certain embodiments, R⁶ is alkyl.

In certain embodiments, R⁶ is haloalkyl.

In certain embodiments, R⁶ is alkenyl.

In certain embodiments, R⁶ is alkynyl.

In certain embodiments, R⁶ is aryl.

In certain embodiments, R⁶ is heteroaryl.

In certain embodiments, R⁶ is cycloalkyl.

### Non-Limiting Embodiments of R⁷

In certain embodiments, R⁷ is hydrogen.

In certain embodiments, R⁷ is alkyl.

In certain embodiments, R⁷ is haloalkyl.

In certain embodiments, R⁷ is alkenyl.

In certain embodiments, R⁷ is alkynyl.

In certain embodiments, R⁷ is aryl.

In certain embodiments, R⁷ is heteroaryl.

In certain embodiments, R⁷ is cycloalkyl.

In certain embodiments, R⁷ is —NR¹R¹².

In certain embodiments, R⁷ is —OR¹²

In certain embodiments, R⁷ is SR¹².

### Non-Limiting Embodiments of R⁸

In certain embodiments, R⁸ is hydrogen.

In certain embodiments, R⁸ is alkyl.

In certain embodiments, R⁸ is haloalkyl.

In certain embodiments, R⁸ is alkenyl.

In certain embodiments, R⁸ is alkynyl.

In certain embodiments, R⁸ is aryl.

In certain embodiments, R⁸ is heteroaryl.

In certain embodiments, R⁸ is cycloalkyl.

In certain embodiments, R⁸ is —NR¹R¹².

In certain embodiments, R⁸ is —OR¹².

In certain embodiments, R⁸ is SR¹².

### Non-Limiting Embodiments of R¹⁰

In certain embodiments, R¹⁰ is hydrogen.

In certain embodiments, R¹⁰ is halogen.

In certain embodiments, R¹⁰ is alkyl.

In certain embodiments, R¹⁰ is haloalkyl.

In certain embodiments, R¹⁰ is cyano.

In certain embodiments, R¹⁰ is nitro.

In certain embodiments, R¹⁰ is alkenyl.

In certain embodiments, R¹⁰ is alkynyl.

In certain embodiments, R¹⁰ is aryl.

In certain embodiments, R¹⁰ is heteroaryl.

In certain embodiments, R¹⁰ is cycloalkyl.

In certain embodiments, R¹⁰ is —C(O)R⁷.

In certain embodiments, R¹⁰ is —NR²C(O)R⁷.

In certain embodiments, R¹⁰ is —OC(O)R⁷.

In certain embodiments, R¹⁰ is —NR¹R².

In certain embodiments, R¹⁰ is —OR².

In certain embodiments, R¹⁰ is SR².

In certain embodiments, R¹⁰ is —S(O)R⁷.

In certain embodiments, R¹⁰ is S(O)₂R⁷.

In certain embodiments, R¹⁰ is —P(O)R⁷R⁸.

### Non-Limiting Embodiments of R^{10b}:

In certain embodiments, R^{10b} is hydrogen.

In certain embodiments, R^{10b} is halogen.

In certain embodiments, R^{10b} is alkyl.

In certain embodiments, R^{10b} is haloalkyl.

In certain embodiments, R^{10b} is cyano.

In certain embodiments, R^{10b} is nitro.

In certain embodiments, R^{10b} is alkenyl.

In certain embodiments, R^{10b} is alkynyl.

In certain embodiments, R^{10b} is aryl.

In certain embodiments, R^{10b} is heteroaryl.

In certain embodiments, R^{10b} is cycloalkyl.

In certain embodiments, R^{10b} is —C(O)R⁷.

In certain embodiments, R^{10b} is —NR²C(O)R⁷.

In certain embodiments, R^{10b} is —OC(O)R⁷.

In certain embodiments, R^{10b} is —NR¹R².

In certain embodiments, R^{10b} is —OR².

In certain embodiments, R^{10b} is SR².

In certain embodiments, R^{10b} is —S(O)R⁷.

In certain embodiments, R^{10b} is S(O)₂R⁷.

In certain embodiments, R^{10b} is —P(O)R⁷R⁸.

Non-limiting embodiments of R¹¹:

In certain embodiments, R¹¹ is

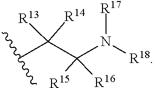

In certain embodiments, R¹¹ is

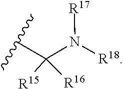

In certain embodiments, R¹¹ is

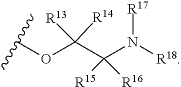

In certain embodiments, R¹¹ is

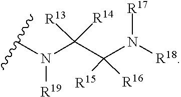

In certain embodiments, R¹¹ is

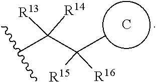

In certain embodiments, R¹¹ is

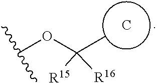

In certain embodiments, R¹¹ is

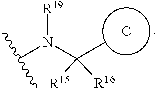

In certain embodiments, R¹¹ is

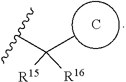

In certain embodiments, R¹¹ is

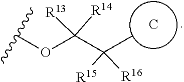

In certain embodiments, R¹¹ is

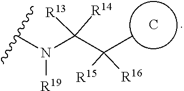

### Non-Limiting Embodiments of R¹²

In certain embodiments, R¹² is hydrogen.

In certain embodiments, R¹² is alkyl.

In certain embodiments, R¹² is haloalkyl.

In certain embodiments, R¹² is alkenyl.

In certain embodiments, R¹² is alkynyl.

In certain embodiments, R¹² is aryl.

In certain embodiments, R¹² is heteroaryl.

In certain embodiments, R¹² is cycloalkyl.

### Non-Limiting Embodiments of R¹³

In certain embodiments, R¹³ is hydrogen.

In certain embodiments, R¹³ is alkyl.

In certain embodiments, R¹³ is haloalkyl.

In certain embodiments, R¹³ is alkenyl.

In certain embodiments, R¹³ is alkynyl.

In certain embodiments, R¹³ is aryl.

In certain embodiments, R¹³ is heteroaryl.

In certain embodiments, R¹³ is cycloalkyl.

### Non-Limiting Embodiments of R¹⁴

In certain embodiments, R¹⁴ is hydrogen.

In certain embodiments, R¹⁴ is alkyl.

In certain embodiments, R¹⁴ is haloalkyl.

In certain embodiments, R¹⁴ is alkenyl.

In certain embodiments, R¹⁴ is alkynyl.

In certain embodiments, R¹⁴ is aryl.

In certain embodiments, R¹⁴ is heteroaryl.

In certain embodiments, R¹⁴ is cycloalkyl.

### Non-Limiting Embodiments of R¹⁵

In certain embodiments, R¹⁵ is hydrogen.

In certain embodiments, R¹⁵ is alkyl.

In certain embodiments, R¹⁵ is haloalkyl.

In certain embodiments, R¹⁵ is alkenyl.

In certain embodiments, R¹⁵ is alkynyl.

In certain embodiments, R¹⁵ is aryl.

In certain embodiments, R¹⁵ is heteroaryl.

In certain embodiments, R¹⁵ is cycloalkyl.

### Non-Limiting Embodiments of R¹⁶

In certain embodiments, R¹⁶ is hydrogen.

In certain embodiments, R¹⁶ is alkyl.

In certain embodiments, R¹⁶ is haloalkyl.

In certain embodiments, R¹⁶ is alkenyl.

In certain embodiments, R¹⁶ is alkynyl.

In certain embodiments, R¹⁶ is aryl.

In certain embodiments, R¹⁶ is heteroaryl.

In certain embodiments, R¹⁶ is cycloalkyl.

### Non-Limiting Embodiments of R^{16b}

In certain embodiments, R^{16b} is alkyl.

In certain embodiments, R^{16b} is haloalkyl.

In certain embodiments, R^{16b} is alkenyl.

In certain embodiments, R^{16b} is alkynyl.

In certain embodiments, R^{16b} is aryl.

In certain embodiments, R^{16b} is heteroaryl.

In certain embodiments, R^{16b} is cycloalkyl.

### Non-Limiting Embodiments of R¹⁷

In certain embodiments, R¹⁷ is hydrogen.

In certain embodiments, R¹⁷ is alkyl.

In certain embodiments, R¹⁷ is haloalkyl.

In certain embodiments, R¹⁷ is alkenyl.

In certain embodiments, R¹⁷ is alkynyl.

In certain embodiments, R¹⁷ is heteroaryl.

In certain embodiments, R¹⁷ is -alkylcycloalkyl.

In certain embodiments, R¹⁷ is —CH₂CH₂—OR².

In certain embodiments, R¹⁷ is —CH₂CH₂—NR¹R².

In certain embodiments, R¹⁷ is -alkyl-aryl.

In certain embodiments, R¹⁷ is -alkyl-heteroaryl.

In certain embodiments, R¹⁷ is cycloalkyl.

### Non-Limiting Embodiments of R¹⁸

In certain embodiments, R¹⁸ is hydrogen.

In certain embodiments, R¹⁸ is alkyl.

In certain embodiments, R¹⁸ is haloalkyl.

In certain embodiments, R¹⁸ is alkenyl.

In certain embodiments, R¹⁸ is aryl.

In certain embodiments, R¹⁸ is heteroaryl.

In certain embodiments, R¹⁸ is cycloalkyl.

In certain embodiments, R¹⁸ is —C(O)R⁷.

### Non-Limiting Embodiments of R¹⁹

In certain embodiments, R¹⁹ is hydrogen.

In certain embodiments, R¹⁹ is alkyl.

In certain embodiments, R¹⁹ is haloalkyl.

In certain embodiments, R¹⁹ is alkenyl.

In certain embodiments, R¹⁹ is alkynyl.

In certain embodiments, R¹⁹ is aryl.

In certain embodiments, R¹⁹ is heteroaryl.

In certain embodiments, R¹⁹ is cycloalkyl.

In certain embodiments, R¹⁹ is cycloalkyl.

In certain embodiments, R¹⁹ is —C(O)R⁷.

### Non-Limiting Embodiments of R²⁰

In certain embodiments, R²⁰ is hydrogen.

In certain embodiments, R²⁰ is halogen.

In certain embodiments, R²⁰ is alkyl.

In certain embodiments, R²⁰ is haloalkyl.

In certain embodiments, R²⁰ is cyano.

In certain embodiments, R²⁰ is nitro.

In certain embodiments, R²⁰ is alkenyl.

In certain embodiments, R²⁰ is alkynyl.

In certain embodiments, R²⁰ is aryl.

In certain embodiments, R²⁰ is heteroaryl.

In certain embodiments, R²⁰ is cycloalkyl.

In certain embodiments, R²⁰ is —C(O)R⁷.

In certain embodiments, R²⁰ is —NR²C(O)R⁷.

In certain embodiments, R²⁰ is —OC(O)R⁷.

In certain embodiments, R²⁰ is —NR¹R².

In certain embodiments, R²⁰ is —OR².

In certain embodiments, R²⁰ is SR².

In certain embodiments, R²⁰ is S(O)R⁷.

In certain embodiments, R²⁰ is S(O)₂R⁷.

In certain embodiments, R²⁰ is —P(O)R⁷R⁸.

In certain embodiments, R²⁰ is heteroaryl

### Non-Limiting Embodiments of R²¹

In certain embodiments, R²¹ is heteroaryl

In certain embodiments, R²¹ is hydrogen.

In certain embodiments, R²¹ is halogen.

In certain embodiments, R²¹ is alkyl.

In certain embodiments, R²¹ is haloalkyl.

In certain embodiments, R²¹ is cyano.

In certain embodiments, R²¹ is nitro.

In certain embodiments, R²¹ is alkenyl.

In certain embodiments, R²¹ is alkynyl.

In certain embodiments, R²¹ is aryl.

In certain embodiments, R²¹ is heteroaryl.

In certain embodiments, R²¹ is cycloalkyl.

In certain embodiments, R²¹ is —C(O)R⁷.

In certain embodiments, R²¹ is —NR²C(O)R⁷.

In certain embodiments, R²¹ is —OC(O)R⁷.

In certain embodiments, R²¹ is —NR¹R².

In certain embodiments, R²¹ is —OR².

In certain embodiments, R²¹ is SR².

In certain embodiments, R²¹ is S(O)R⁷.

In certain embodiments, R²¹ is S(O)₂R⁷.

In certain embodiments, R²¹ is —P(O)R⁷R⁸.

In certain embodiments, R²¹ is heteroaryl.

### Non-Limiting Embodiments of R²²

In certain embodiments, R²² is hydrogen.

In certain embodiments, R²² is halogen.

In certain embodiments, R²² is alkyl.

In certain embodiments, R²² is haloalkyl.

In certain embodiments, R²² is cyano.

In certain embodiments, R²² is nitro.

In certain embodiments, R²² is alkenyl.

In certain embodiments, R²² is alkynyl.

In certain embodiments, R²² is aryl.

In certain embodiments, R²² is heteroaryl.

In certain embodiments, R²² is cycloalkyl.

In certain embodiments, R²² is —C(O)R⁷.

In certain embodiments, R²² is —NR²C(O)R⁷.

In certain embodiments, R²² is —OC(O)R⁷.

In certain embodiments, R²² is —NR¹R².

In certain embodiments, R²² is —OR².

In certain embodiments, R²² is SR².

In certain embodiments, R²² is S(O)R⁷.

In certain embodiments, R²² is S(O)₂R⁷.

In certain embodiments, R²² is —P(O)R⁷R⁸.

### REPRESENTATIVE COMPOUNDS OF THE PRESENT INVENTION

| Compd No. | Example No. | Structure |
|---|---|---|
| 172 | 1 | 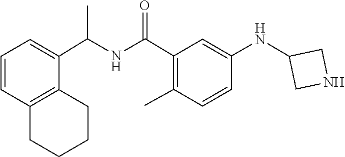 [SMILES: Cc1ccc(NC2CNC2)cc1C(=O)NC(C)c1cccc2c1CCCC2] |
| 168 | 2 | 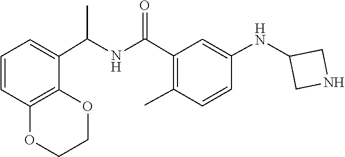 [SMILES: Cc1ccc(NC2CNC2)cc1C(=O)NC(C)c1cccc2c1OCCO2] |
| 155 | 3 | 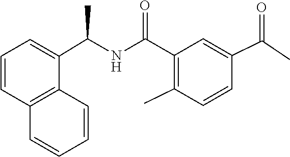 [SMILES: CC(=O)c1ccc(C)c(C(=O)N[C@H](C)c2cccc3ccccc23)c1] |
| 101 | 4 | 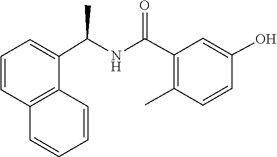 [SMILES: Cc1ccc(O)cc1C(=O)N[C@H](C)c1cccc2ccccc12] |
| 136 | 5 | 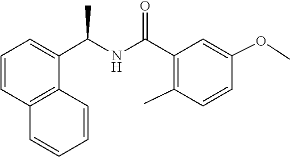 [SMILES: COc1ccc(C)c(C(=O)N[C@H](C)c2cccc3ccccc23)c1] |
| 132 | 6 | 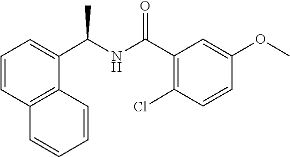 [SMILES: COc1ccc(Cl)c(C(=O)N[C@H](C)c2cccc3ccccc23)c1] |
| 158 | 7 | 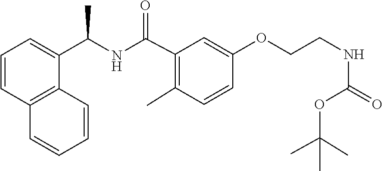 [SMILES: Cc1ccc(OCCNC(=O)OC(C)(C)C)cc1C(=O)N[C@H](C)c1cccc2ccccc12] |
| 159 | 8 | 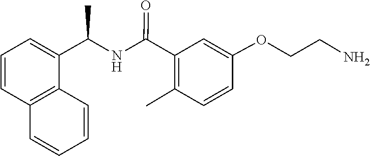 [SMILES: Cc1ccc(OCCN)cc1C(=O)N[C@H](C)c1cccc2ccccc12] |
| 160 | 9 | 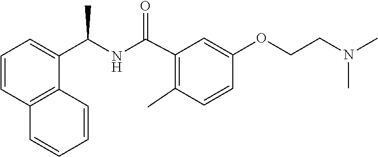 [SMILES: Cc1ccc(OCCN(C)C)cc1C(=O)N[C@H](C)c1cccc2ccccc12] |
| 152 | 10 | 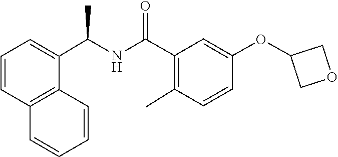 [SMILES: Cc1ccc(OC2COC2)cc1C(=O)N[C@H](C)c1cccc2ccccc12] |
| 141 | 11 | 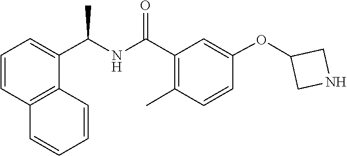 [SMILES: Cc1ccc(OC2CNC2)cc1C(=O)N[C@H](C)c1cccc2ccccc12] |
| 164 | 12 | 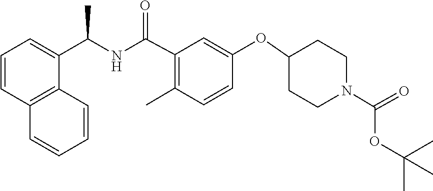 [SMILES: Cc1ccc(OC2CCN(C(=O)OC(C)(C)C)CC2)cc1C(=O)N[C@H](C)c1cccc2ccccc12] |
| 162 | 13 | 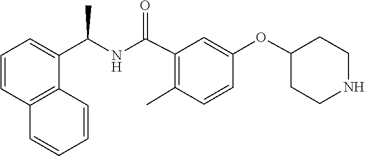 [SMILES: Cc1ccc(OC2CCNCC2)cc1C(=O)N[C@H](C)c1cccc2ccccc12] |
| 100 | 14 | 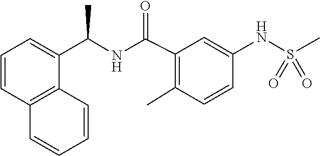 [SMILES: Cc1ccc(NS(C)(=O)=O)cc1C(=O)N[C@H](C)c1cccc2ccccc12] |
| 121 | 15 | 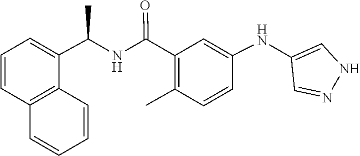 [SMILES: Cc1ccc(Nc2cn[nH]c2)cc1C(=O)N[C@H](C)c1cccc2ccccc12] |
| 107 | 16 | 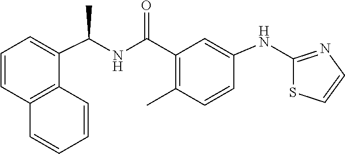 [SMILES: Cc1ccc(Nc2nccs2)cc1C(=O)N[C@H](C)c1cccc2ccccc12] |
| 115 | 17 | 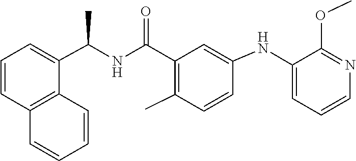 [SMILES: COc1ncccc1Nc1ccc(C)c(C(=O)N[C@H](C)c2cccc3ccccc23)c1] |
| 119 | 18 | 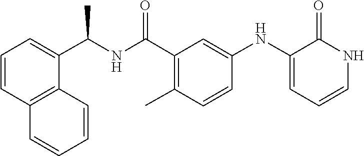 [SMILES: Cc1ccc(Nc2ccc[nH]c2=O)cc1C(=O)N[C@H](C)c1cccc2ccccc12] |
| 165 | 19 | 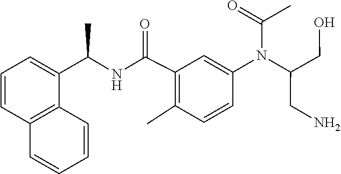 [SMILES: CC(=O)N(c1ccc(C)c(C(=O)N[C@H](C)c2cccc3ccccc23)c1)C(CN)CO] |
| 153 | 20 | 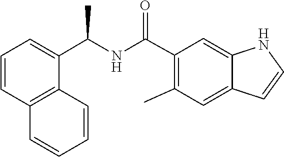 [SMILES: Cc1cc2cc[nH]c2cc1C(=O)N[C@H](C)c1cccc2ccccc12] |
| 189 | 21 | 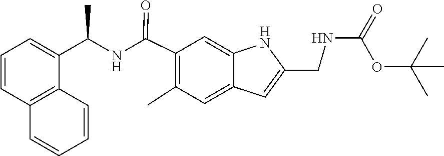 [SMILES: Cc1cc2cc(CNC(=O)OC(C)(C)C)[nH]c2cc1C(=O)N[C@H](C)c1cccc2ccccc12] |
| 148 | 22 | 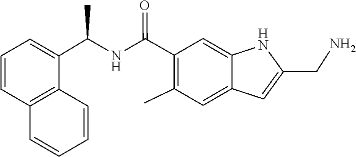 [SMILES: Cc1cc2cc(CN)[nH]c2cc1C(=O)N[C@H](C)c1cccc2ccccc12] |
| 156 | 23 | 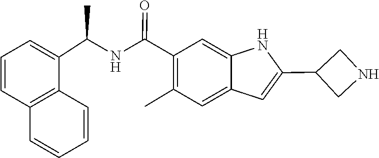 [SMILES: Cc1cc2cc(C3CNC3)[nH]c2cc1C(=O)N[C@H](C)c1cccc2ccccc12] |
| 192 | 24 | 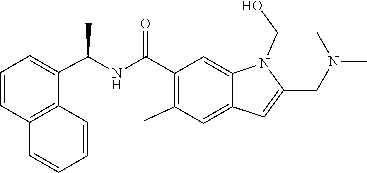 [SMILES: Cc1cc2cc(CN(C)C)n(CO)c2cc1C(=O)N[C@H](C)c1cccc2ccccc12] |
| 206 | 25 | 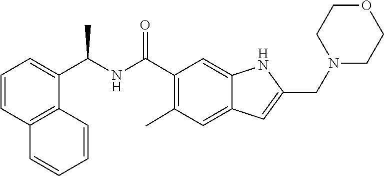 [SMILES: Cc1cc2cc(CN3CCOCC3)[nH]c2cc1C(=O)N[C@H](C)c1cccc2ccccc12] |
| 125 | 26 | 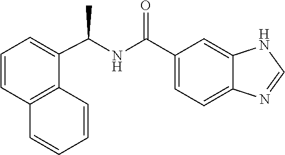 [SMILES: C[C@@H](NC(=O)c1ccc2nc[nH]c2c1)c1cccc2ccccc12] |
| 102 | 27 | 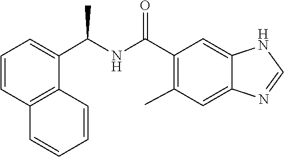 [SMILES: Cc1cc2nc[nH]c2cc1C(=O)N[C@H](C)c1cccc2ccccc12] |
| 128 | 28 | 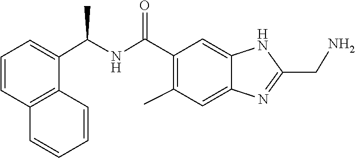 [SMILES: Cc1cc2nc(CN)[nH]c2cc1C(=O)N[C@H](C)c1cccc2ccccc12] |
| 131 | 29 | 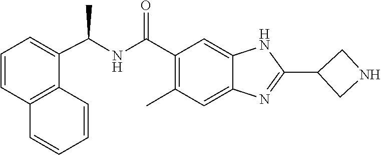 [SMILES: Cc1cc2nc(C3CNC3)[nH]c2cc1C(=O)N[C@H](C)c1cccc2ccccc12] |
| 124 | 30 | 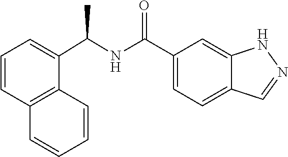 [SMILES: C[C@@H](NC(=O)c1ccc2cn[nH]c2c1)c1cccc2ccccc12] |
| 142 | 31 | 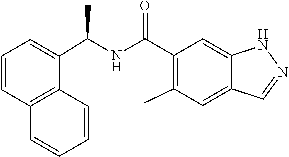 [SMILES: Cc1cc2cn[nH]c2cc1C(=O)N[C@H](C)c1cccc2ccccc12] |
| 150 | 32 | 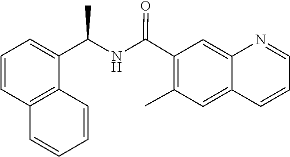 [SMILES: Cc1cc2cccnc2cc1C(=O)N[C@H](C)c1cccc2ccccc12] |
| 167 | 33 | 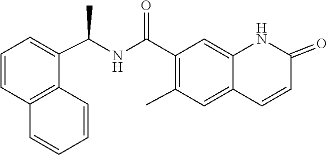 [SMILES: Cc1cc2ccc(=O)[nH]c2cc1C(=O)N[C@H](C)c1cccc2ccccc12] |
| 145 | 34 | 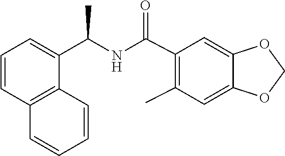 [SMILES: Cc1cc2c(cc1C(=O)N[C@H](C)c1cccc3ccccc13)OCO2] |
| 163 | 35 | 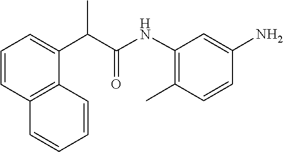 [SMILES: Cc1ccc(N)cc1NC(=O)C(C)c1cccc2ccccc12] |
| 195 | 36 | 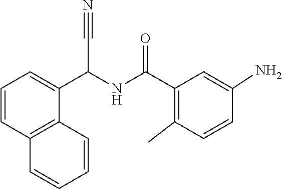 [SMILES: Cc1ccc(N)cc1C(=O)NC(C#N)c1cccc2ccccc12] |
| 200 | 37 | 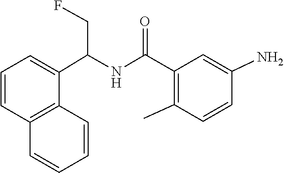 [SMILES: Cc1ccc(N)cc1C(=O)NC(CF)c1cccc2ccccc12] |
| 185 | 38 | 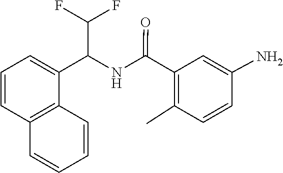 [SMILES: Cc1ccc(N)cc1C(=O)NC(c1cccc2ccccc12)C(F)F] |
| 183 | 39 | 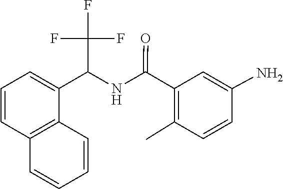 [SMILES: Cc1ccc(N)cc1C(=O)NC(c1cccc2ccccc12)C(F)(F)F] |
| 191 | 40 | 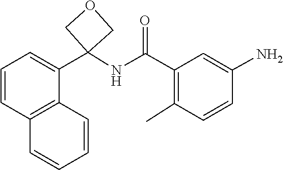 [SMILES: Cc1ccc(N)cc1C(=O)NC1(c2cccc3ccccc23)COC1] |
| 281 | 41 | 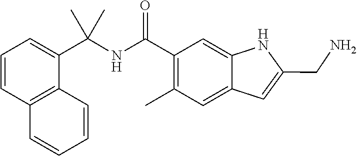 [SMILES: Cc1cc2cc(CN)[nH]c2cc1C(=O)NC(C)(C)c1cccc2ccccc12] |
| 241 | 42 | 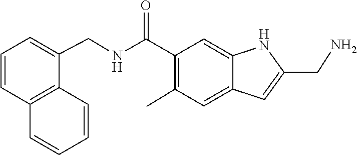 [SMILES: Cc1cc2cc(CN)[nH]c2cc1C(=O)NCc1cccc2ccccc12] |
| 147 | 43 | 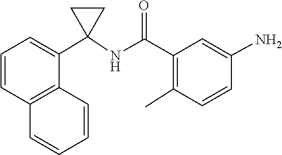 [SMILES: Cc1ccc(N)cc1C(=O)NC1(c2cccc3ccccc23)CC1] |
| 219 | 44 | 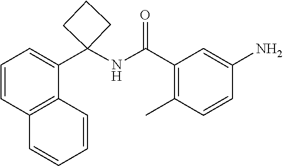 [SMILES: Cc1ccc(N)cc1C(=O)NC1(c2cccc3ccccc23)CCC1] |
| 177 | 45 | 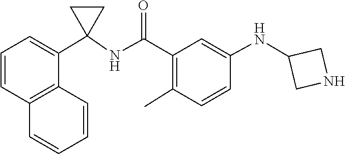 [SMILES: Cc1ccc(NC2CNC2)cc1C(=O)NC1(c2cccc3ccccc23)CC1] |
| 313 | 46 | 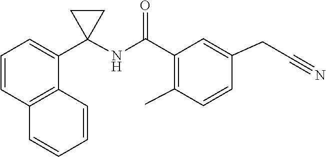 [SMILES: Cc1ccc(CC#N)cc1C(=O)NC1(c2cccc3ccccc23)CC1] |
| 378 | 47 | 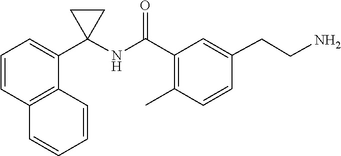 [SMILES: Cc1ccc(CCN)cc1C(=O)NC1(c2cccc3ccccc23)CC1] |
| 257 | 48 | 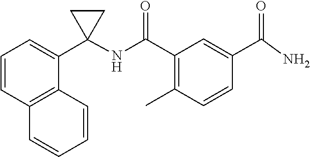 [SMILES: Cc1ccc(C(N)=O)cc1C(=O)NC1(c2cccc3ccccc23)CC1] |
| 312 | 49 | 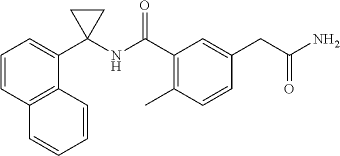 [SMILES: Cc1ccc(CC(N)=O)cc1C(=O)NC1(c2cccc3ccccc23)CC1] |
| 398 | 50 | 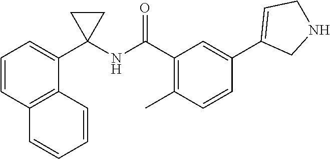 [SMILES: Cc1ccc(C2=CCNC2)cc1C(=O)NC1(c2cccc3ccccc23)CC1] |
| 399 | 51 | 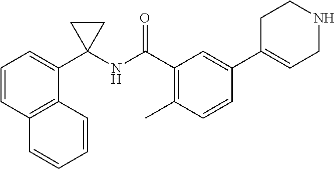 [SMILES: Cc1ccc(C2=CCNCC2)cc1C(=O)NC1(c2cccc3ccccc23)CC1] |
| 373 | 52 | 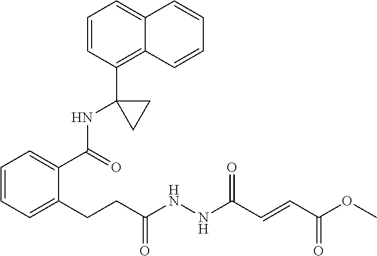 [SMILES: COC(=O)/C=C/C(=O)NNC(=O)CCc1ccccc1C(=O)NC1(c2cccc3ccccc23)CC1] |
| 215 | 53 | 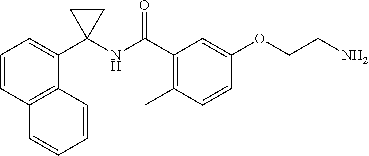 [SMILES: Cc1ccc(OCCN)cc1C(=O)NC1(c2cccc3ccccc23)CC1] |
| 211 | 54 | 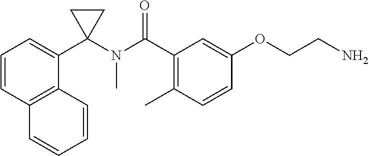 [SMILES: Cc1ccc(OCCN)cc1C(=O)N(C)C1(c2cccc3ccccc23)CC1] |
| 316 | 55 | 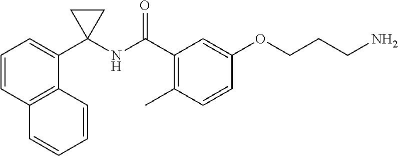 [SMILES: Cc1ccc(OCCCN)cc1C(=O)NC1(c2cccc3ccccc23)CC1] |
| 233 | 56 | 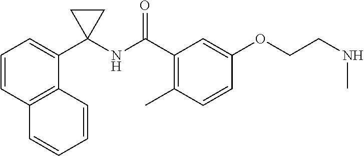 [SMILES: CNCCOc1ccc(C)c(C(=O)NC2(c3cccc4ccccc34)CC2)c1] |
| 386 | 57 | 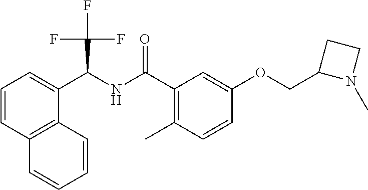 [SMILES: Cc1ccc(OCC2CCN2C)cc1C(=O)N[C@@H](c1cccc2ccccc12)C(F)(F)F] |
| 214 | 58 | 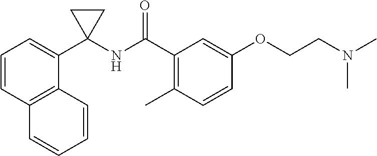 [SMILES: Cc1ccc(OCCN(C)C)cc1C(=O)NC1(c2cccc3ccccc23)CC1] |
| 351 | 59 | 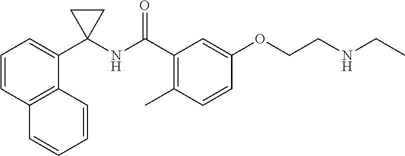 [SMILES: CCNCCOc1ccc(C)c(C(=O)NC2(c3cccc4ccccc34)CC2)c1] |
| 247 | 60 | 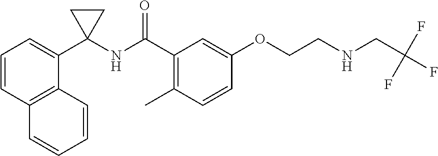 [SMILES: Cc1ccc(OCCNCC(F)(F)F)cc1C(=O)NC1(c2cccc3ccccc23)CC1] |
| 362 | 61 | 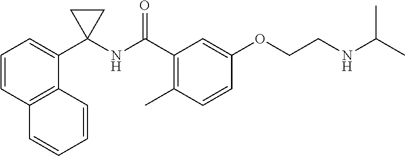 [SMILES: Cc1ccc(OCCNC(C)C)cc1C(=O)NC1(c2cccc3ccccc23)CC1] |
| 361 | 62 | 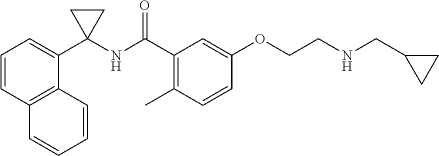 [SMILES: Cc1ccc(OCCNCC2CC2)cc1C(=O)NC1(c2cccc3ccccc23)CC1] |
| 315 | 63 | 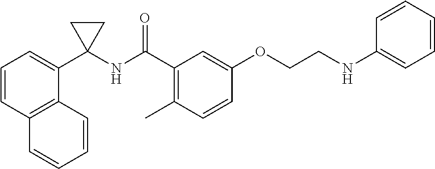 [SMILES: Cc1ccc(OCCNc2ccccc2)cc1C(=O)NC1(c2cccc3ccccc23)CC1] |
| 360 | 64 | 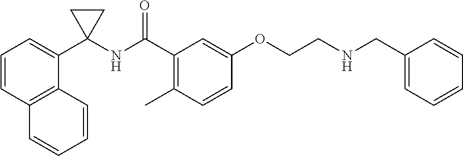 [SMILES: Cc1ccc(OCCNCc2ccccc2)cc1C(=O)NC1(c2cccc3ccccc23)CC1] |
| 359 | 65 | 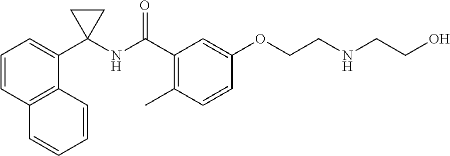 [SMILES: Cc1ccc(OCCNCCO)cc1C(=O)NC1(c2cccc3ccccc23)CC1] |
| 358 | 66 | 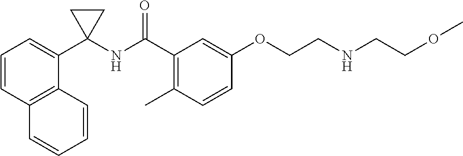 [SMILES: COCCNCCOc1ccc(C)c(C(=O)NC2(c3cccc4ccccc34)CC2)c1] |
| 300 | 67 | 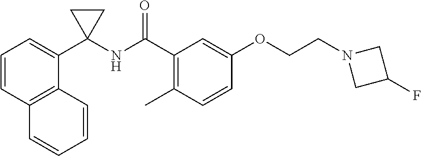 [SMILES: Cc1ccc(OCCN2CC(F)C2)cc1C(=O)NC1(c2cccc3ccccc23)CC1] |
| 254 | 68 | 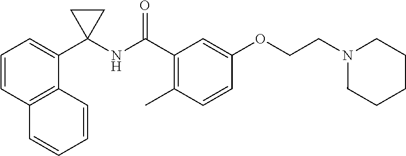 [SMILES: Cc1ccc(OCCN2CCCCC2)cc1C(=O)NC1(c2cccc3ccccc23)CC1] |
| 284 | 69 | 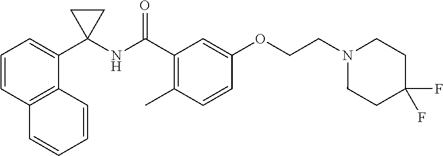 [SMILES: Cc1ccc(OCCN2CCC(F)(F)CC2)cc1C(=O)NC1(c2cccc3ccccc23)CC1] |
| 209 | 70 | 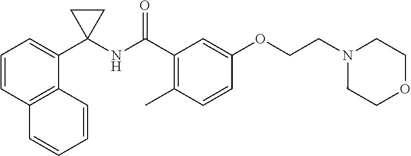 [SMILES: Cc1ccc(OCCN2CCOCC2)cc1C(=O)NC1(c2cccc3ccccc23)CC1] |
| 324 | 71 | 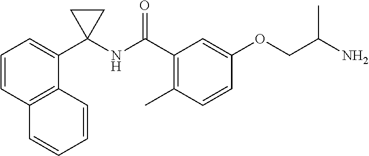 [SMILES: Cc1ccc(OCC(C)N)cc1C(=O)NC1(c2cccc3ccccc23)CC1] |
| 343 | 72 | 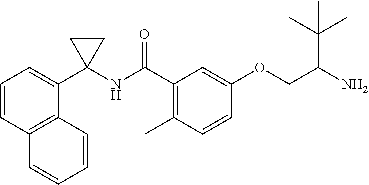 [SMILES: Cc1ccc(OCC(N)C(C)(C)C)cc1C(=O)NC1(c2cccc3ccccc23)CC1] |
| 400 | 73 | 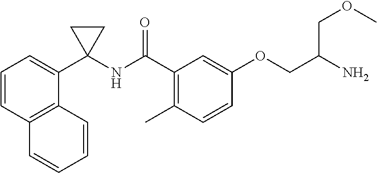 [SMILES: COCC(N)COc1ccc(C)c(C(=O)NC2(c3cccc4ccccc34)CC2)c1] |
| 367 | 74 | 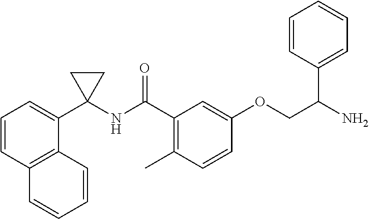 [SMILES: Cc1ccc(OCC(N)c2ccccc2)cc1C(=O)NC1(c2cccc3ccccc23)CC1] |
| 322 | 75 | 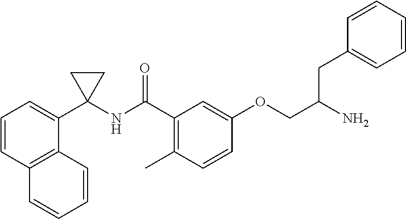 [SMILES: Cc1ccc(OCC(N)Cc2ccccc2)cc1C(=O)NC1(c2cccc3ccccc23)CC1] |
| 352 | 76 | 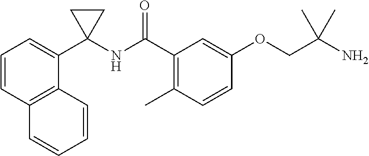 [SMILES: Cc1ccc(OCC(C)(C)N)cc1C(=O)NC1(c2cccc3ccccc23)CC1] |
| 256 | 77 | 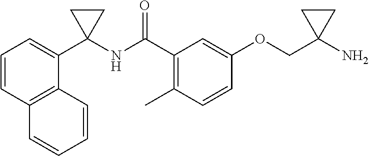 [SMILES: Cc1ccc(OCC2(N)CC2)cc1C(=O)NC1(c2cccc3ccccc23)CC1] |
| 274 | 78 | 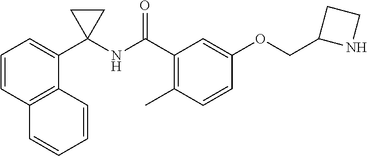 [SMILES: Cc1ccc(OCC2CCN2)cc1C(=O)NC1(c2cccc3ccccc23)CC1] |
| 310 | 79 | 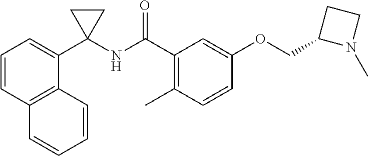 [SMILES: Cc1ccc(OC[C@@H]2CCN2C)cc1C(=O)NC1(c2cccc3ccccc23)CC1] |
| 309 | 80 | 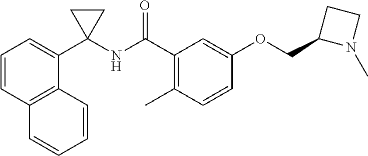 [SMILES: Cc1ccc(OC[C@H]2CCN2C)cc1C(=O)NC1(c2cccc3ccccc23)CC1] |
| 387 | 81 | 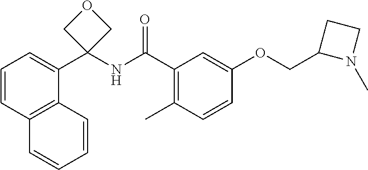 [SMILES: Cc1ccc(OCC2CCN2C)cc1C(=O)NC1(c2cccc3ccccc23)COC1] |
| 349 | 82 | 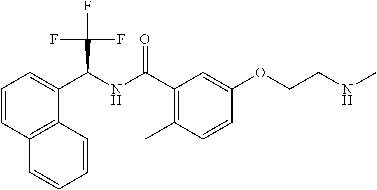 [SMILES: CNCCOc1ccc(C)c(C(=O)N[C@@H](c2cccc3ccccc23)C(F)(F)F)c1] |
| 314 | 83 | 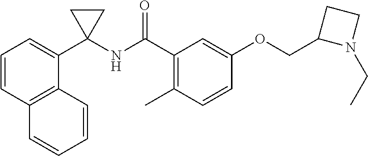 [SMILES: CCN1CCC1COc1ccc(C)c(C(=O)NC2(c3cccc4ccccc34)CC2)c1] |
| 396 | 84 | 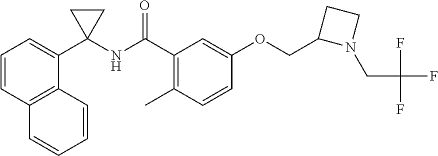 [SMILES: Cc1ccc(OCC2CCN2CC(F)(F)F)cc1C(=O)NC1(c2cccc3ccccc23)CC1] |
| 331 | 85 | 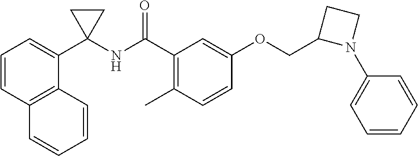 [SMILES: Cc1ccc(OCC2CCN2c2ccccc2)cc1C(=O)NC1(c2cccc3ccccc23)CC1] |
| 330 | 86 | 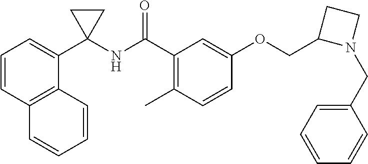 [SMILES: Cc1ccc(OCC2CCN2Cc2ccccc2)cc1C(=O)NC1(c2cccc3ccccc23)CC1] |
| 323 | 87 | 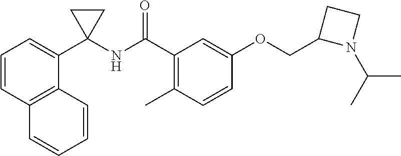 [SMILES: Cc1ccc(OCC2CCN2C(C)C)cc1C(=O)NC1(c2cccc3ccccc23)CC1] |
| 332 | 88 | 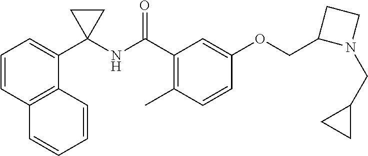 [SMILES: Cc1ccc(OCC2CCN2CC2CC2)cc1C(=O)NC1(c2cccc3ccccc23)CC1] |
| 348 | 89 | 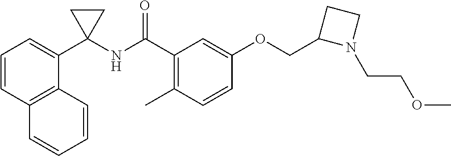 [SMILES: COCCN1CCC1COc1ccc(C)c(C(=O)NC2(c3cccc4ccccc34)CC2)c1] |
| 341 | 90 | 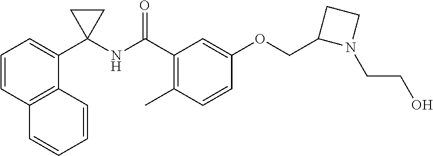 [SMILES: Cc1ccc(OCC2CCN2CCO)cc1C(=O)NC1(c2cccc3ccccc23)CC1] |
| 394 | 91 | 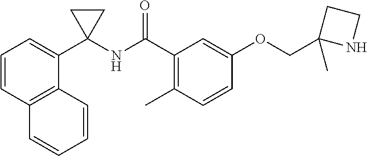 [SMILES: Cc1ccc(OCC2(C)CCN2)cc1C(=O)NC1(c2cccc3ccccc23)CC1] |
| 393 | 92 | 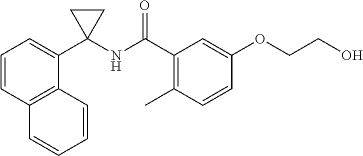 [SMILES: Cc1ccc(OCCO)cc1C(=O)NC1(c2cccc3ccccc23)CC1] |
| 285 | 93 | 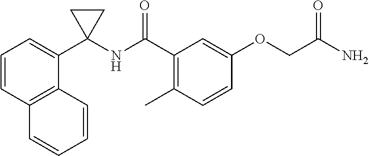 [SMILES: Cc1ccc(OCC(N)=O)cc1C(=O)NC1(c2cccc3ccccc23)CC1] |
| 171 | 94 | 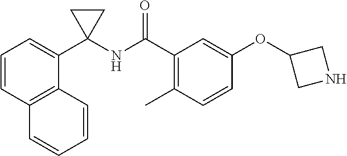 [SMILES: Cc1ccc(OC2CNC2)cc1C(=O)NC1(c2cccc3ccccc23)CC1] |
| 265 | 95 | 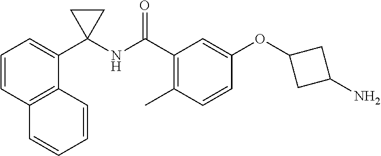 [SMILES: Cc1ccc(OC2CC(N)C2)cc1C(=O)NC1(c2cccc3ccccc23)CC1] |
| 263 | 96 | 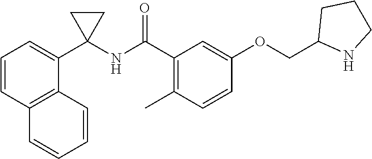 [SMILES: Cc1ccc(OCC2CCCN2)cc1C(=O)NC1(c2cccc3ccccc23)CC1] |
| 283 | 97 | 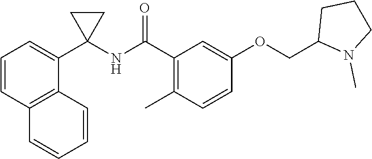 [SMILES: Cc1ccc(OCC2CCCN2C)cc1C(=O)NC1(c2cccc3ccccc23)CC1] |
| 268 | 98 | 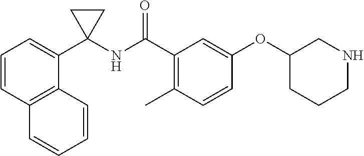 [SMILES: Cc1ccc(OC2CCCNC2)cc1C(=O)NC1(c2cccc3ccccc23)CC1] |
| 289 | 99 | 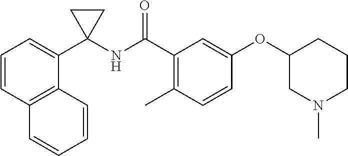 [SMILES: Cc1ccc(OC2CCCN(C)C2)cc1C(=O)NC1(c2cccc3ccccc23)CC1] |
| 339 | 100 | 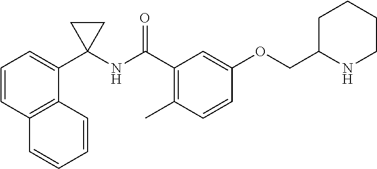 [SMILES: Cc1ccc(OCC2CCCCN2)cc1C(=O)NC1(c2cccc3ccccc23)CC1] |
| 288 | 101 | 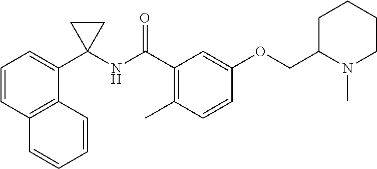 [SMILES: Cc1ccc(OCC2CCCCN2C)cc1C(=O)NC1(c2cccc3ccccc23)CC1] |
| 293 | 101 | 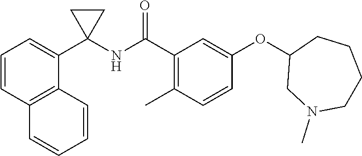 [SMILES: Cc1ccc(OC2CCCCN(C)C2)cc1C(=O)NC1(c2cccc3ccccc23)CC1] |
| 286 | 102 | 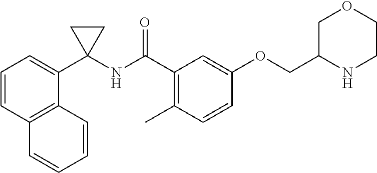 [SMILES: Cc1ccc(OCC2COCCN2)cc1C(=O)NC1(c2cccc3ccccc23)CC1] |
| 302 | 103 | 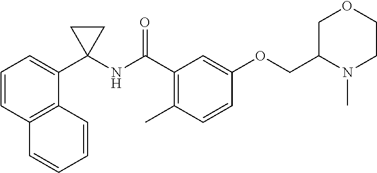 [SMILES: Cc1ccc(OCC2COCCN2C)cc1C(=O)NC1(c2cccc3ccccc23)CC1] |
| 376 | 105 | 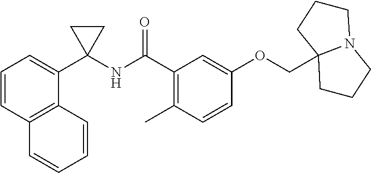 [SMILES: Cc1ccc(OCC23CCCN2CCC3)cc1C(=O)NC1(c2cccc3ccccc23)CC1] |
| 303 | 106 | 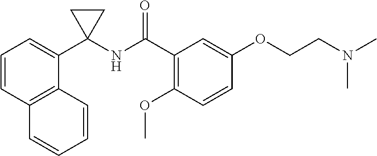 [SMILES: COc1ccc(OCCN(C)C)cc1C(=O)NC1(c2cccc3ccccc23)CC1] |
| 269 | 107 | 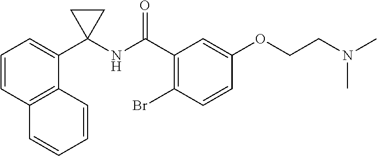 [SMILES: CN(C)CCOc1ccc(Br)c(C(=O)NC2(c3cccc4ccccc34)CC2)c1] |
| 337 | 108 | 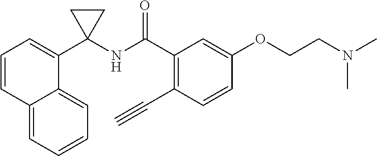 [SMILES: C#Cc1ccc(OCCN(C)C)cc1C(=O)NC1(c2cccc3ccccc23)CC1] |
| 353 | 109 | 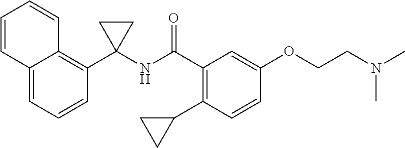 [SMILES: CN(C)CCOc1ccc(C2CC2)c(C(=O)NC2(c3cccc4ccccc34)CC2)c1] |
| 311 | 110 | 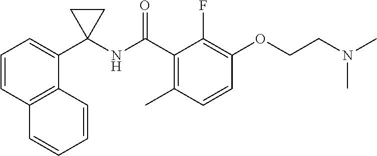 [SMILES: Cc1ccc(OCCN(C)C)c(F)c1C(=O)NC1(c2cccc3ccccc23)CC1] |
| 216 | 111 | 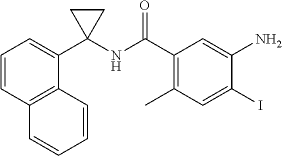 [SMILES: Cc1cc(I)c(N)cc1C(=O)NC1(c2cccc3ccccc23)CC1] |
| 369 | 112 | 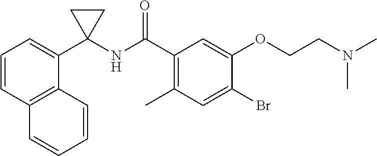 [SMILES: Cc1cc(Br)c(OCCN(C)C)cc1C(=O)NC1(c2cccc3ccccc23)CC1] |
| 366 | 113 | 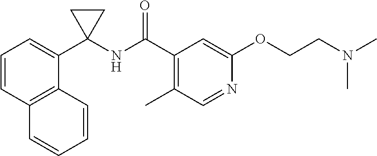 [SMILES: Cc1cnc(OCCN(C)C)cc1C(=O)NC1(c2cccc3ccccc23)CC1] |
| 350 | 114 | 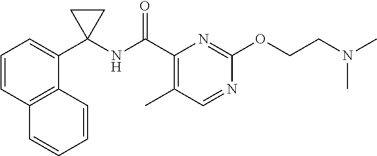 [SMILES: Cc1cnc(OCCN(C)C)nc1C(=O)NC1(c2cccc3ccccc23)CC1] |
| 275 | 115 | 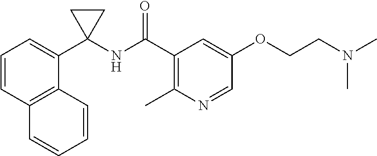 [SMILES: Cc1ncc(OCCN(C)C)cc1C(=O)NC1(c2cccc3ccccc23)CC1] |
| 199 | 116 | 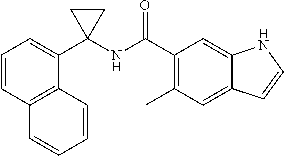 [SMILES: Cc1cc2cc[nH]c2cc1C(=O)NC1(c2cccc3ccccc23)CC1] |
| 240 | 117 | 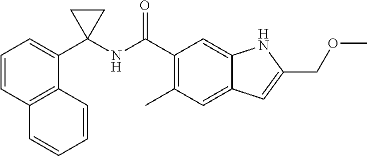 [SMILES: COCc1cc2cc(C)c(C(=O)NC3(c4cccc5ccccc45)CC3)cc2[nH]1] |
| 180 | 118 | 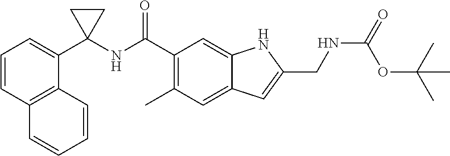 [SMILES: Cc1cc2cc(CNC(=O)OC(C)(C)C)[nH]c2cc1C(=O)NC1(c2cccc3ccccc23)CC1] |
| 170 | 119 | 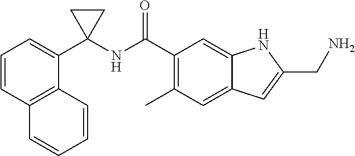 [SMILES: Cc1cc2cc(CN)[nH]c2cc1C(=O)NC1(c2cccc3ccccc23)CC1] |
| 248 | 120 | 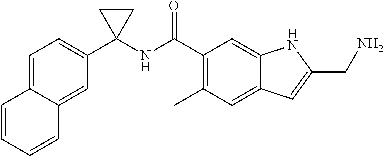 [SMILES: Cc1cc2cc(CN)[nH]c2cc1C(=O)NC1(c2ccc3ccccc3c2)CC1] |
| 246 | 121 | 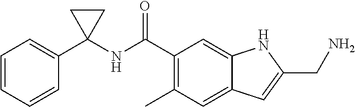 [SMILES: Cc1cc2cc(CN)[nH]c2cc1C(=O)NC1(c2ccccc2)CC1] |
| 244 | 122 | 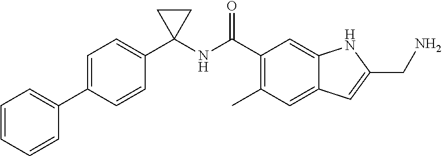 [SMILES: Cc1cc2cc(CN)[nH]c2cc1C(=O)NC1(c2ccc(-c3ccccc3)cc2)CC1] |
| 232 | 123 | 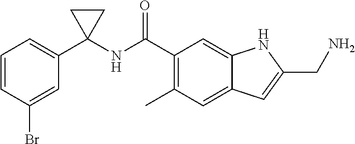 [SMILES: Cc1cc2cc(CN)[nH]c2cc1C(=O)NC1(c2cccc(Br)c2)CC1] |
| 221 | 124 | 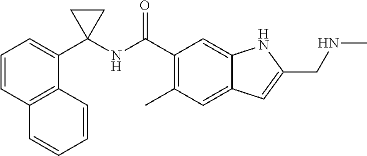 [SMILES: CNCc1cc2cc(C)c(C(=O)NC3(c4cccc5ccccc45)CC3)cc2[nH]1] |
| 173 | 125 | 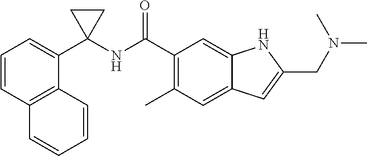 [SMILES: Cc1cc2cc(CN(C)C)[nH]c2cc1C(=O)NC1(c2cccc3ccccc23)CC1] |
| 273 | 126 | 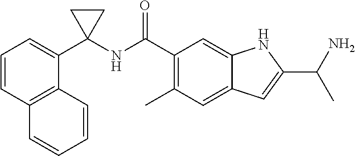 [SMILES: Cc1cc2cc(C(C)N)[nH]c2cc1C(=O)NC1(c2cccc3ccccc23)CC1] |
| 260 | 127 | 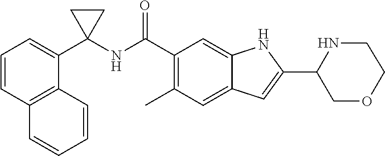 [SMILES: Cc1cc2cc(C3COCCN3)[nH]c2cc1C(=O)NC1(c2cccc3ccccc23)CC1] |
| 202 | 128 | 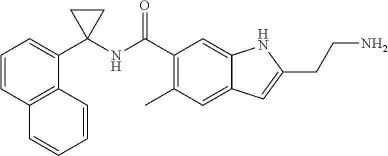 [SMILES: Cc1cc2cc(CCN)[nH]c2cc1C(=O)NC1(c2cccc3ccccc23)CC1] |
| 222 | 129 | 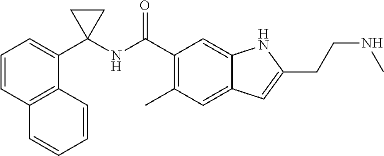 [SMILES: CNCCc1cc2cc(C)c(C(=O)NC3(c4cccc5ccccc45)CC3)cc2[nH]1] |
| 213 | 130 | 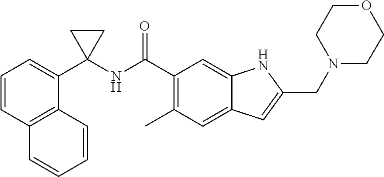 [SMILES: Cc1cc2cc(CN3CCOCC3)[nH]c2cc1C(=O)NC1(c2cccc3ccccc23)CC1] |
| 229 | 131 | 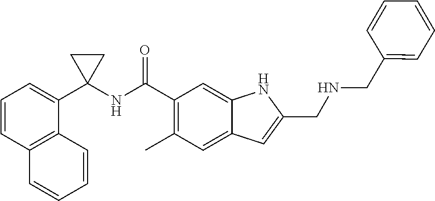 [SMILES: Cc1cc2cc(CNCc3ccccc3)[nH]c2cc1C(=O)NC1(c2cccc3ccccc23)CC1] |
| 204 | 132 | 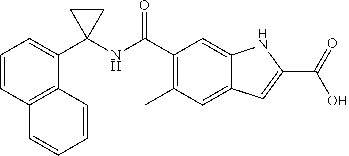 [SMILES: Cc1cc2cc(C(=O)O)[nH]c2cc1C(=O)NC1(c2cccc3ccccc23)CC1] |
| 205 | 133 | 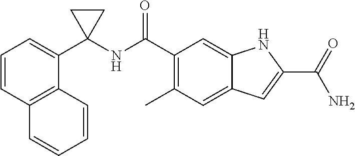 [SMILES: Cc1cc2cc(C(N)=O)[nH]c2cc1C(=O)NC1(c2cccc3ccccc23)CC1] |
| 225 | 134 | 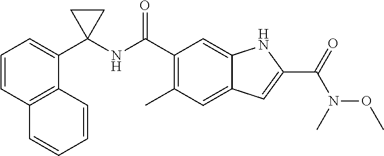 [SMILES: CON(C)C(=O)c1cc2cc(C)c(C(=O)NC3(c4cccc5ccccc45)CC3)cc2[nH]1] |
| 226 | 135 | 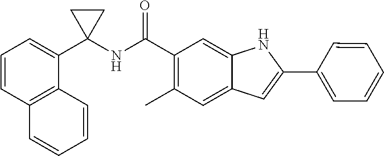 [SMILES: Cc1cc2cc(-c3ccccc3)[nH]c2cc1C(=O)NC1(c2cccc3ccccc23)CC1] |
| 234 | 136 | 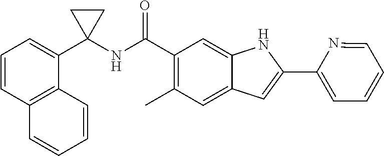 [SMILES: Cc1cc2cc(-c3ccccn3)[nH]c2cc1C(=O)NC1(c2cccc3ccccc23)CC1] |
| 201 | 137 | 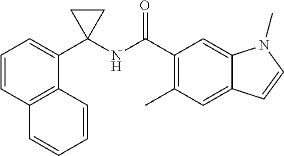 [SMILES: Cc1cc2ccn(C)c2cc1C(=O)NC1(c2cccc3ccccc23)CC1] |
| 296 | 138 | 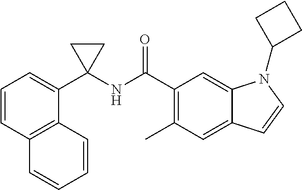 [SMILES: Cc1cc2ccn(C3CCC3)c2cc1C(=O)NC1(c2cccc3ccccc23)CC1] |
| 218 | 139 | 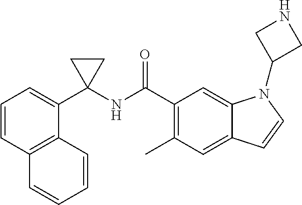 [SMILES: Cc1cc2ccn(C3CNC3)c2cc1C(=O)NC1(c2cccc3ccccc23)CC1] |
| 290 | 140 | 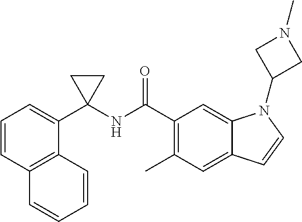 [SMILES: Cc1cc2ccn(C3CN(C)C3)c2cc1C(=O)NC1(c2cccc3ccccc23)CC1] |
| 239 | 141 | 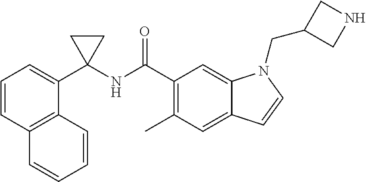 [SMILES: Cc1cc2ccn(CC3CNC3)c2cc1C(=O)NC1(c2cccc3ccccc23)CC1] |
| 295 | 142 | 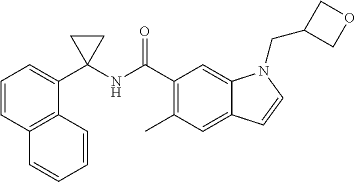 [SMILES: Cc1cc2ccn(CC3COC3)c2cc1C(=O)NC1(c2cccc3ccccc23)CC1] |
| 203 | 143 | 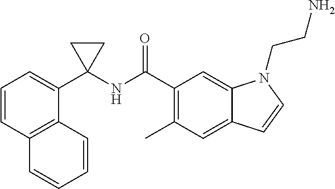 [SMILES: Cc1cc2ccn(CCN)c2cc1C(=O)NC1(c2cccc3ccccc23)CC1] |
| 212 | 144 | 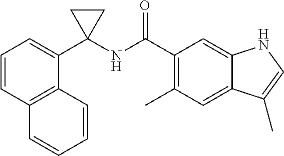 [SMILES: Cc1cc2c(C)c[nH]c2cc1C(=O)NC1(c2cccc3ccccc23)CC1] |
| 292 | 145 | 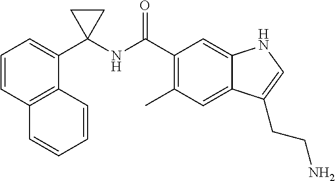 [SMILES: Cc1cc2c(CCN)c[nH]c2cc1C(=O)NC1(c2cccc3ccccc23)CC1] |
| 207 | 146 | 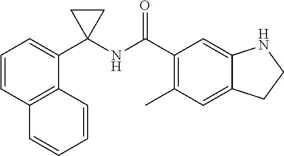 [SMILES: Cc1cc2c(cc1C(=O)NC1(c3cccc4ccccc34)CC1)NCC2] |
| 210 | 147 | 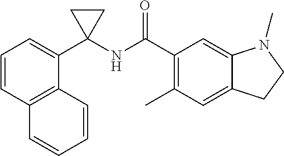 [SMILES: Cc1cc2c(cc1C(=O)NC1(c3cccc4ccccc34)CC1)N(C)CC2] |
| 249 | 148 | 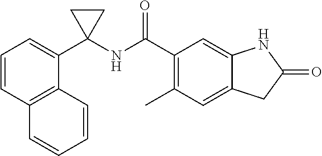 [SMILES: Cc1cc2c(cc1C(=O)NC1(c3cccc4ccccc34)CC1)NC(=O)C2] |
| 243 | 149 | 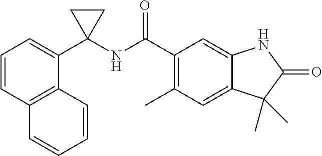 [SMILES: Cc1cc2c(cc1C(=O)NC1(c3cccc4ccccc34)CC1)NC(=O)C2(C)C] |
| 262 | 150 | 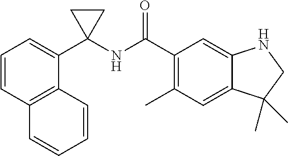 [SMILES: Cc1cc2c(cc1C(=O)NC1(c3cccc4ccccc34)CC1)NCC2(C)C] |
| 190 | 151 | 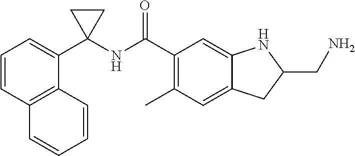 [SMILES: Cc1cc2c(cc1C(=O)NC1(c3cccc4ccccc34)CC1)NC(CN)C2] |
| 231 | 152 | 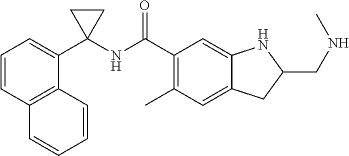 [SMILES: CNCC1Cc2cc(C)c(C(=O)NC3(c4cccc5ccccc45)CC3)cc2N1] |
| 176 | 153 | 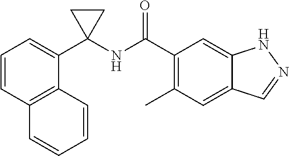 [SMILES: Cc1cc2cn[nH]c2cc1C(=O)NC1(c2cccc3ccccc23)CC1] |
| 304 | 154 | 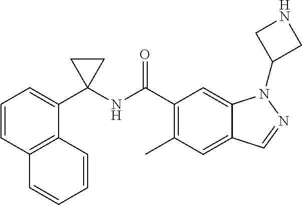 [SMILES: Cc1cc2cnn(C3CNC3)c2cc1C(=O)NC1(c2cccc3ccccc23)CC1] |
| 305 | 154 | 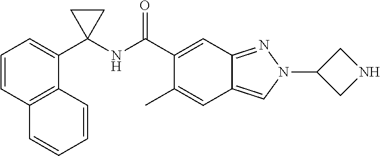 [SMILES: Cc1cc2cn(C3CNC3)nc2cc1C(=O)NC1(c2cccc3ccccc23)CC1] |
| 197 | 155 | 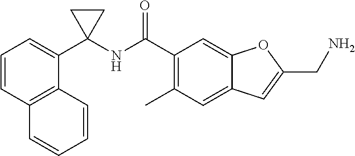 [SMILES: Cc1cc2cc(CN)oc2cc1C(=O)NC1(c2cccc3ccccc23)CC1] |
| 198 | 156 | 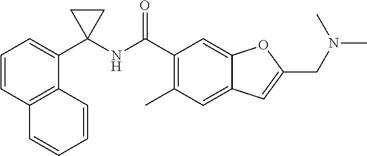 [SMILES: Cc1cc2cc(CN(C)C)oc2cc1C(=O)NC1(c2cccc3ccccc23)CC1] |
| 220 | 157 | 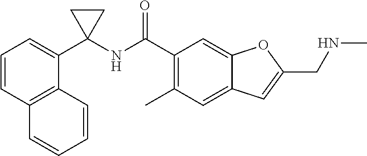 [SMILES: CNCc1cc2cc(C)c(C(=O)NC3(c4cccc5ccccc45)CC3)cc2o1] |
| 235 | 158 | 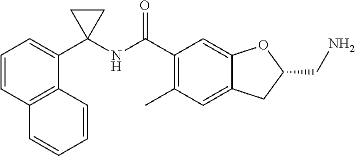 [SMILES: Cc1cc2c(cc1C(=O)NC1(c3cccc4ccccc34)CC1)O[C@H](CN)C2] |
| 236 | 158 | 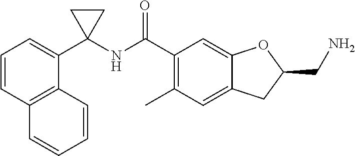 [SMILES: Cc1cc2c(cc1C(=O)NC1(c3cccc4ccccc34)CC1)O[C@@H](CN)C2] |
| 230 | 159 | 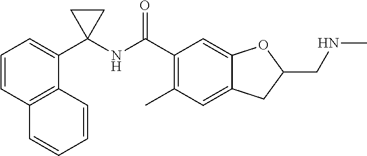 [SMILES: CNCC1Cc2cc(C)c(C(=O)NC3(c4cccc5ccccc45)CC3)cc2O1] |
| 224 | 160 | 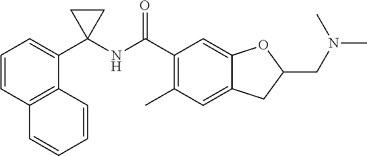 [SMILES: Cc1cc2c(cc1C(=O)NC1(c3cccc4ccccc34)CC1)OC(CN(C)C)C2] |
| 397 | 161 | 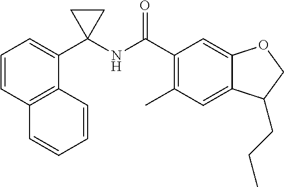 [SMILES: CCCC1COc2cc(C(=O)NC3(c4cccc5ccccc45)CC3)c(C)cc21] |
| 223 | 162 | 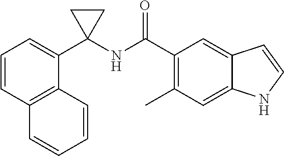 [SMILES: Cc1cc2[nH]ccc2cc1C(=O)NC1(c2cccc3ccccc23)CC1] |
| 280 | 163 | 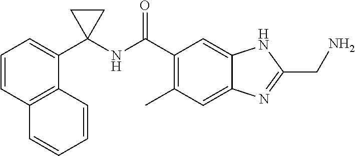 [SMILES: Cc1cc2nc(CN)[nH]c2cc1C(=O)NC1(c2cccc3ccccc23)CC1] |
| 245 | 164 | 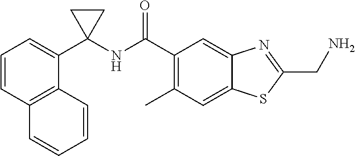 [SMILES: Cc1cc2sc(CN)nc2cc1C(=O)NC1(c2cccc3ccccc23)CC1] |
| 238 | 165 | 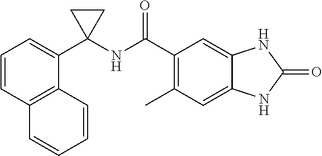 [SMILES: Cc1cc2[nH]c(=O)[nH]c2cc1C(=O)NC1(c2cccc3ccccc23)CC1] |
| 237 | 166 | 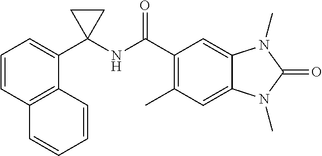 [SMILES: Cc1cc2c(cc1C(=O)NC1(c3cccc4ccccc34)CC1)n(C)c(=O)n2C] |
| 297 | 167 | 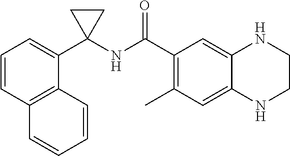 [SMILES: Cc1cc2c(cc1C(=O)NC1(c3cccc4ccccc34)CC1)NCCN2] |
| 227 | 168 | 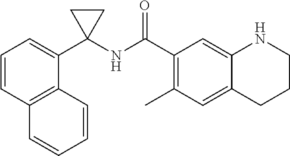 [SMILES: Cc1cc2c(cc1C(=O)NC1(c3cccc4ccccc34)CC1)NCCC2] |
| 334 | 169 | 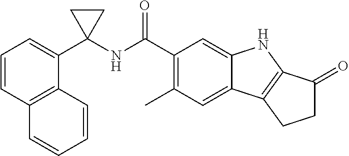 [SMILES: Cc1cc2c3c([nH]c2cc1C(=O)NC1(c2cccc4ccccc24)CC1)C(=O)CC3] |
| 194 | 170 | 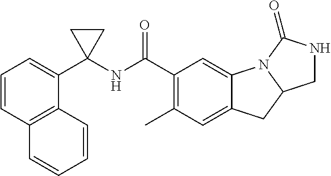 [SMILES: Cc1cc2c(cc1C(=O)NC1(c3cccc4ccccc34)CC1)N1C(=O)NCC1C2] |
| 272 | 171 | 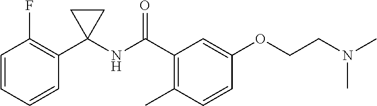 [SMILES: Cc1ccc(OCCN(C)C)cc1C(=O)NC1(c2ccccc2F)CC1] |
| 252 | 172 | 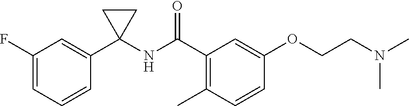 [SMILES: Cc1ccc(OCCN(C)C)cc1C(=O)NC1(c2cccc(F)c2)CC1] |
| 259 | 173 | 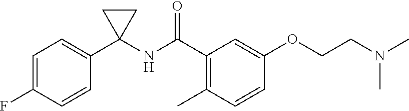 [SMILES: Cc1ccc(OCCN(C)C)cc1C(=O)NC1(c2ccc(F)cc2)CC1] |
| 251 | 174 | 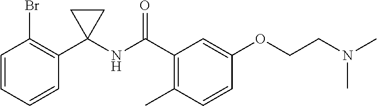 [SMILES: Cc1ccc(OCCN(C)C)cc1C(=O)NC1(c2ccccc2Br)CC1] |
| 261 | 175 | 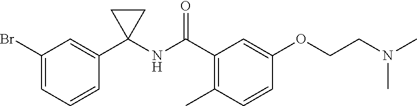 [SMILES: Cc1ccc(OCCN(C)C)cc1C(=O)NC1(c2cccc(Br)c2)CC1] |
| 258 | 176 | 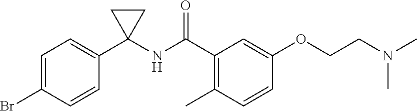 [SMILES: Cc1ccc(OCCN(C)C)cc1C(=O)NC1(c2ccc(Br)cc2)CC1] |
| 326 | 177 | 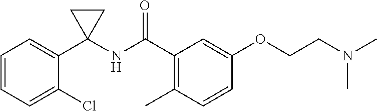 [SMILES: Cc1ccc(OCCN(C)C)cc1C(=O)NC1(c2ccccc2Cl)CC1] |
| 282 | 178 | 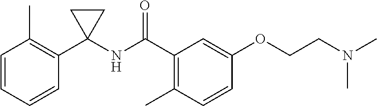 [SMILES: Cc1ccc(OCCN(C)C)cc1C(=O)NC1(c2ccccc2C)CC1] |
| 255 | 179 | 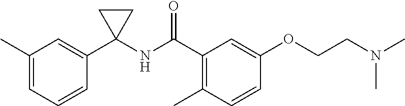 |
| 253 | 180 | 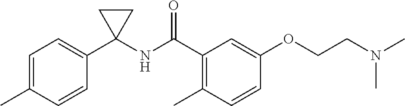 |
| 321 | 181 | 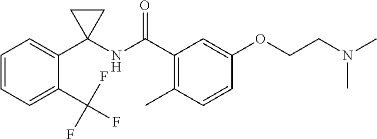 [SMILES: Cc1ccc(OCCN(C)C)cc1C(=O)NC1(c2ccccc2C(F)(F)F)CC1] |
| 364 | 182 | 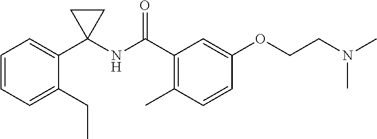 [SMILES: CCc1ccccc1C1(NC(=O)c2cc(OCCN(C)C)ccc2C)CC1] |
| 344 | 183 | 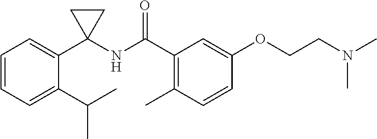 [SMILES: Cc1ccc(OCCN(C)C)cc1C(=O)NC1(c2ccccc2C(C)C)CC1] |
| 267 | 184 | 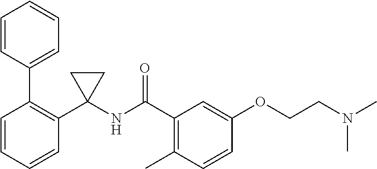 [SMILES: Cc1ccc(OCCN(C)C)cc1C(=O)NC1(c2ccccc2-c2ccccc2)CC1] |
| 294 | 185 | 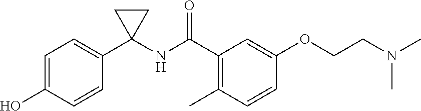 [SMILES: Cc1ccc(OCCN(C)C)cc1C(=O)NC1(c2ccc(O)cc2)CC1] |
| 271 | 186 | 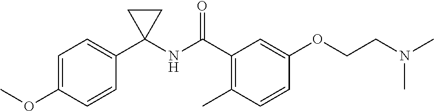 |
| 298 | 187 | 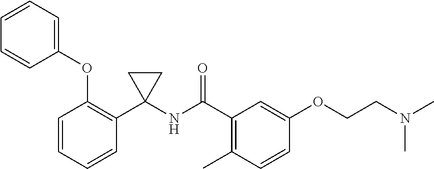 [SMILES: Cc1ccc(OCCN(C)C)cc1C(=O)NC1(c2ccccc2Oc2ccccc2)CC1] |
| 270 | 188 | 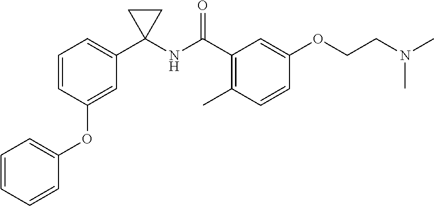 [SMILES: Cc1ccc(OCCN(C)C)cc1C(=O)NC1(c2cccc(Oc3ccccc3)c2)CC1] |
| 279 | 189 | 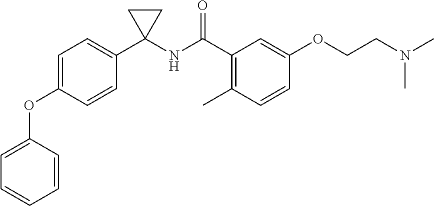 [SMILES: Cc1ccc(OCCN(C)C)cc1C(=O)NC1(c2ccc(Oc3ccccc3)cc2)CC1] |
| 327 | 190 | 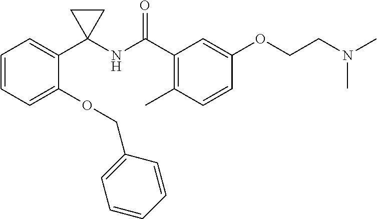 [SMILES: Cc1ccc(OCCN(C)C)cc1C(=O)NC1(c2ccccc2OCc2ccccc2)CC1] |
| 308 | 191 | 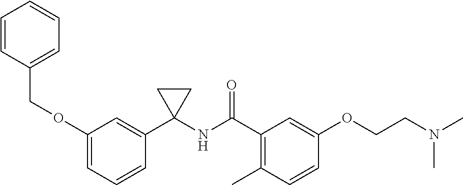 [SMILES: Cc1ccc(OCCN(C)C)cc1C(=O)NC1(c2cccc(OCc3ccccc3)c2)CC1] |
| 307 | 192 | 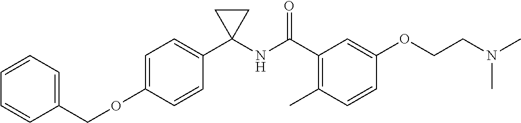 [SMILES: Cc1ccc(OCCN(C)C)cc1C(=O)NC1(c2ccc(OCc3ccccc3)cc2)CC1] |
| 291 | 193 | 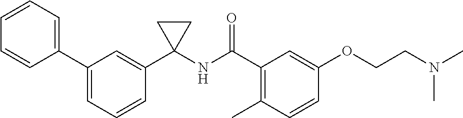 [SMILES: Cc1ccc(OCCN(C)C)cc1C(=O)NC1(c2cccc(-c3ccccc3)c2)CC1] |
| 264 | 194 | 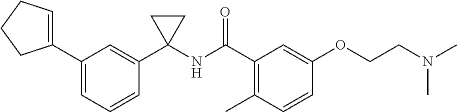 [SMILES: Cc1ccc(OCCN(C)C)cc1C(=O)NC1(c2cccc(C3=CCCC3)c2)CC1] |
| 276 | 195 | 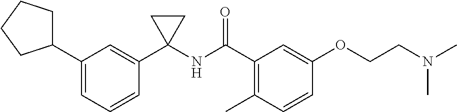 [SMILES: Cc1ccc(OCCN(C)C)cc1C(=O)NC1(c2cccc(C3CCCC3)c2)CC1] |
| 338 | 196 | 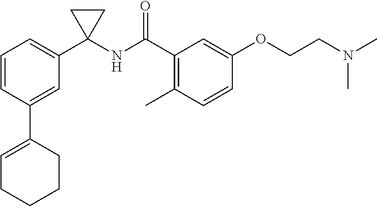 [SMILES: Cc1ccc(OCCN(C)C)cc1C(=O)NC1(c2cccc(C3=CCCCC3)c2)CC1] |
| 266 | 197 | 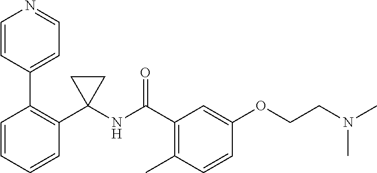 [SMILES: Cc1ccc(OCCN(C)C)cc1C(=O)NC1(c2ccccc2-c2ccncc2)CC1] |
| 355 | 198 | 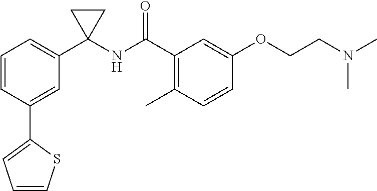 [SMILES: Cc1ccc(OCCN(C)C)cc1C(=O)NC1(c2cccc(-c3cccs3)c2)CC1] |
| 325 | 199 | 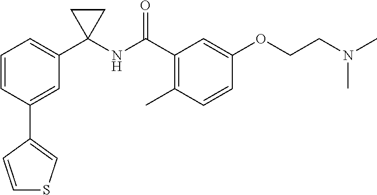 [SMILES: Cc1ccc(OCCN(C)C)cc1C(=O)NC1(c2cccc(-c3ccsc3)c2)CC1] |
| 169 | 200 | 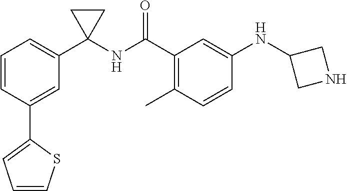 [SMILES: Cc1ccc(NC2CNC2)cc1C(=O)NC1(c2cccc(-c3cccs3)c2)CC1] |
| 187 | 201 | 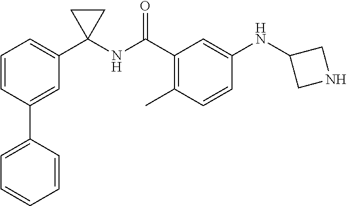 [SMILES: Cc1ccc(NC2CNC2)cc1C(=O)NC1(c2cccc(-c3ccccc3)c2)CC1] |
| 186 | 202 | 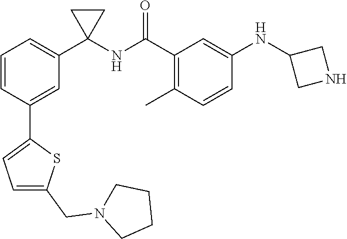 [SMILES: Cc1ccc(NC2CNC2)cc1C(=O)NC1(c2cccc(-c3ccc(CN4CCCC4)s3)c2)CC1] |
| 196 | 203 | 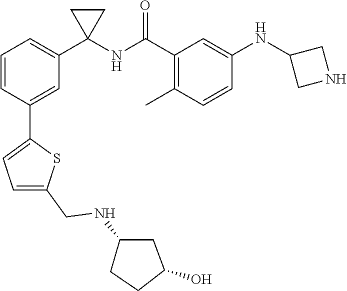 [SMILES: Cc1ccc(NC2CNC2)cc1C(=O)NC1(c2cccc(-c3ccc(CN[C@H]4CC[C@@H](O)C4)s3)c2)CC1] |
| 318 | 204 | 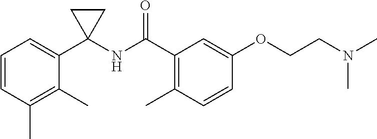 [SMILES: Cc1ccc(OCCN(C)C)cc1C(=O)NC1(c2cccc(C)c2C)CC1] |
| 335 | 205 | 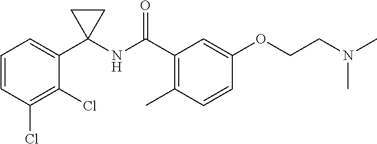 [SMILES: Cc1ccc(OCCN(C)C)cc1C(=O)NC1(c2cccc(Cl)c2Cl)CC1] |
| 317 | 206 | 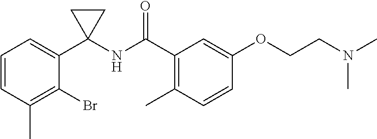 [SMILES: Cc1ccc(OCCN(C)C)cc1C(=O)NC1(c2cccc(C)c2Br)CC1] |
| 336 | 207 | 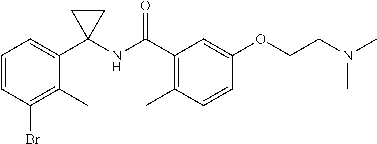 [SMILES: Cc1ccc(OCCN(C)C)cc1C(=O)NC1(c2cccc(Br)c2C)CC1] |
| 340 | 208 | 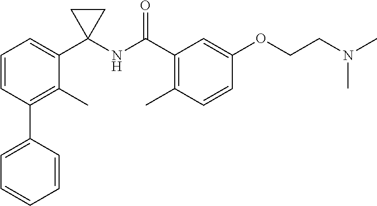 [SMILES: Cc1ccc(OCCN(C)C)cc1C(=O)NC1(c2cccc(-c3ccccc3)c2C)CC1] |
| 329 | 209 | 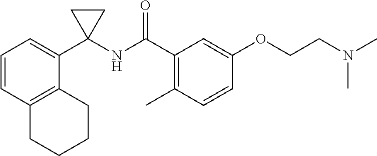 [SMILES: Cc1ccc(OCCN(C)C)cc1C(=O)NC1(c2cccc3c2CCCC3)CC1] |
| 370 | 210 | 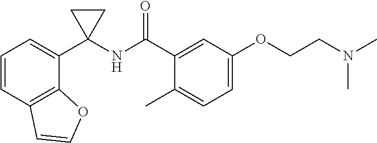 [SMILES: Cc1ccc(OCCN(C)C)cc1C(=O)NC1(c2cccc3ccoc23)CC1] |
| 357 | 211 | 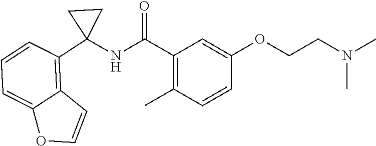 [SMILES: Cc1ccc(OCCN(C)C)cc1C(=O)NC1(c2cccc3occc23)CC1] |
| 368 | 212 | 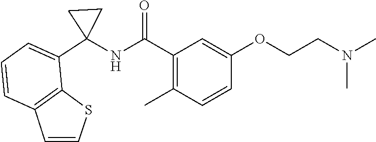 [SMILES: Cc1ccc(OCCN(C)C)cc1C(=O)NC1(c2cccc3ccsc23)CC1] |
| 356 | 213 | 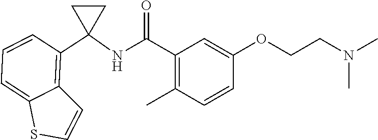 [SMILES: Cc1ccc(OCCN(C)C)cc1C(=O)NC1(c2cccc3sccc23)CC1] |
| 403 | 214 | 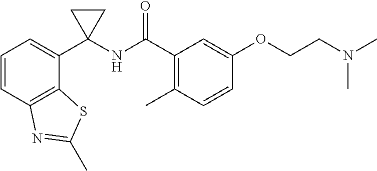 [SMILES: Cc1nc2cccc(C3(NC(=O)c4cc(OCCN(C)C)ccc4C)CC3)c2s1] |
| 346 | 215 | 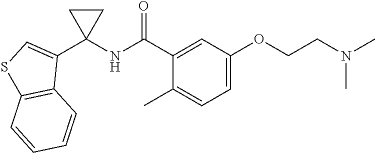 [SMILES: Cc1ccc(OCCN(C)C)cc1C(=O)NC1(c2csc3ccccc23)CC1] |
| 354 | 216 | 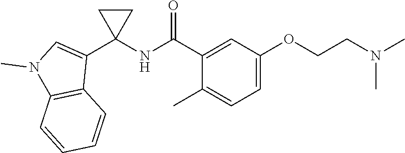 |
| 395 | 217 | 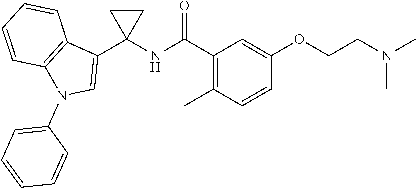 [SMILES: Cc1ccc(OCCN(C)C)cc1C(=O)NC1(c2cn(-c3ccccc3)c3ccccc23)CC1] |
| 301 | 218 | 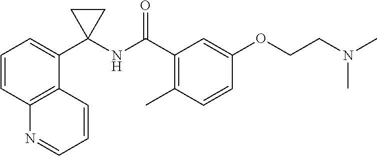 [SMILES: Cc1ccc(OCCN(C)C)cc1C(=O)NC1(c2cccc3ncccc23)CC1] |
| 374 | 219 | 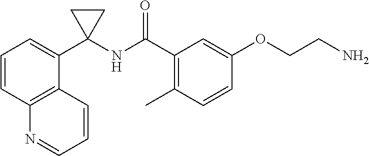 [SMILES: Cc1ccc(OCCN)cc1C(=O)NC1(c2cccc3ncccc23)CC1] |
| 385 | 220 | 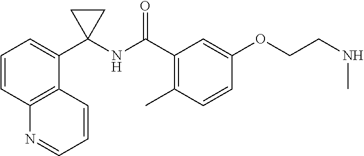 [SMILES: CNCCOc1ccc(C)c(C(=O)NC2(c3cccc4ncccc34)CC2)c1] |
| 381 | 221 | 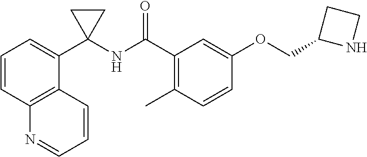 [SMILES: Cc1ccc(OC[C@@H]2CCN2)cc1C(=O)NC1(c2cccc3ncccc23)CC1] |
| 383 | 222 | 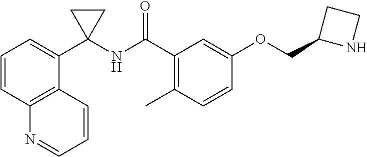 [SMILES: Cc1ccc(OC[C@H]2CCN2)cc1C(=O)NC1(c2cccc3ncccc23)CC1] |
| 391 | 223 | 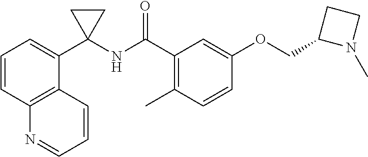 [SMILES: Cc1ccc(OC[C@@H]2CCN2C)cc1C(=O)NC1(c2cccc3ncccc23)CC1] |
| 390 | 224 | 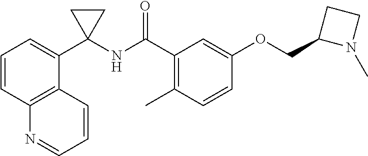 [SMILES: Cc1ccc(OC[C@H]2CCN2C)cc1C(=O)NC1(c2cccc3ncccc23)CC1] |
| 380 | 225 | 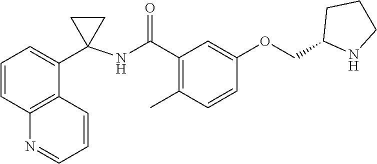 [SMILES: Cc1ccc(OC[C@@H]2CCCN2)cc1C(=O)NC1(c2cccc3ncccc23)CC1] |
| 379 | 226 | 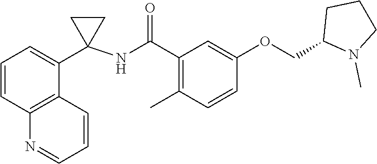 [SMILES: Cc1ccc(OC[C@@H]2CCCN2C)cc1C(=O)NC1(c2cccc3ncccc23)CC1] |
| 371 | 227 | 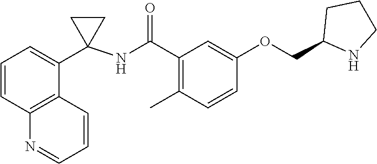 [SMILES: Cc1ccc(OC[C@H]2CCCN2)cc1C(=O)NC1(c2cccc3ncccc23)CC1] |
| 382 | 228 | 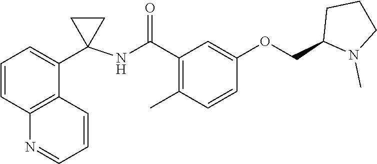 [SMILES: Cc1ccc(OC[C@H]2CCCN2C)cc1C(=O)NC1(c2cccc3ncccc23)CC1] |
| 299 | 229 | 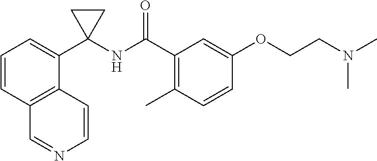 [SMILES: Cc1ccc(OCCN(C)C)cc1C(=O)NC1(c2cccc3cnccc23)CC1] |
| 375 | 230 | 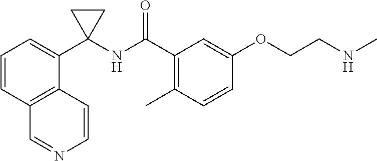 [SMILES: CNCCOc1ccc(C)c(C(=O)NC2(c3cccc4cnccc34)CC2)c1] |
| 402 | 231 | 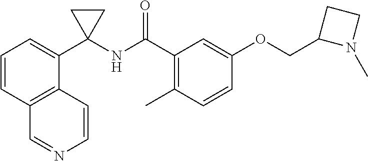 [SMILES: Cc1ccc(OCC2CCN2C)cc1C(=O)NC1(c2cccc3cnccc23)CC1] |
| 333 | 232 | 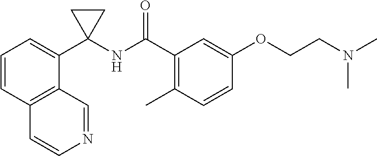 [SMILES: Cc1ccc(OCCN(C)C)cc1C(=O)NC1(c2cccc3ccncc23)CC1] |
| 388 | 233 | 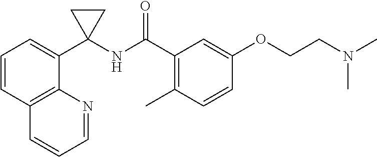 [SMILES: Cc1ccc(OCCN(C)C)cc1C(=O)NC1(c2cccc3cccnc23)CC1] |
| 363 | 234 | 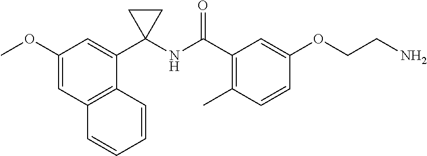 [SMILES: COc1cc(C2(NC(=O)c3cc(OCCN)ccc3C)CC2)c2ccccc2c1] |
| 320 | 235 | 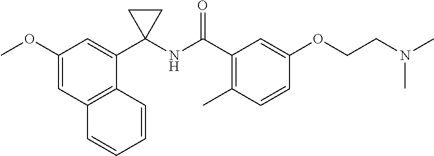 |
| 345 | 236 | 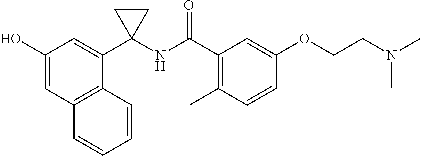 [SMILES: Cc1ccc(OCCN(C)C)cc1C(=O)NC1(c2cc(O)cc3ccccc23)CC1] |
| 347 | 237 | 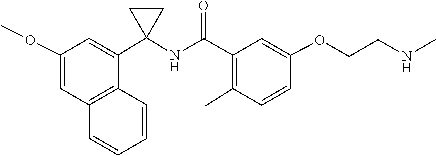 |
| 384 | 238 | 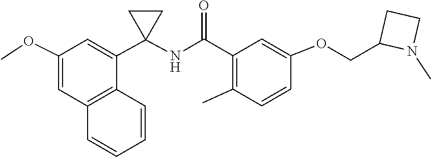 |
| 401 | 239 | 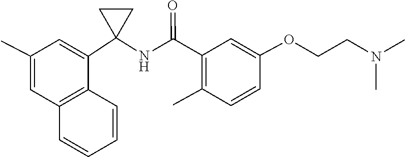 |
| 319 | 240 | 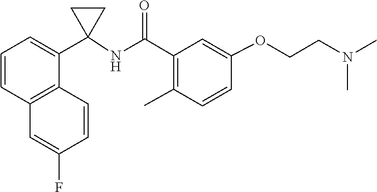 [SMILES: Cc1ccc(OCCN(C)C)cc1C(=O)NC1(c2cccc3cc(F)ccc23)CC1] |
| 392 | 241 | 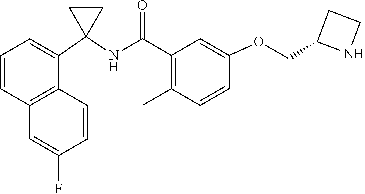 [SMILES: Cc1ccc(OC[C@@H]2CCN2)cc1C(=O)NC1(c2cccc3cc(F)ccc23)CC1] |
| 377 | 242 | 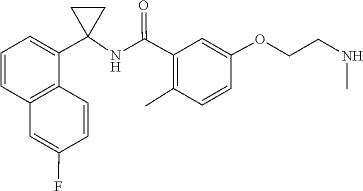 [SMILES: CNCCOc1ccc(C)c(C(=O)NC2(c3cccc4cc(F)ccc34)CC2)c1] |
| 389 | 243 | 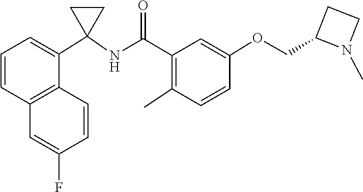 [SMILES: Cc1ccc(OC[C@@H]2CCN2C)cc1C(=O)NC1(c2cccc3cc(F)ccc23)CC1] |
| 365 | 244 | 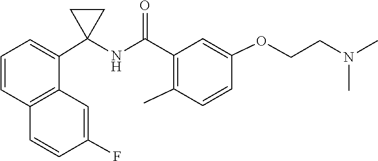 [SMILES: Cc1ccc(OCCN(C)C)cc1C(=O)NC1(c2cccc3ccc(F)cc23)CC1] |
| 120 | 245 | 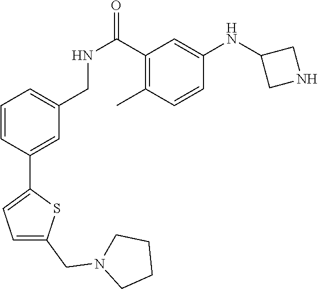 [SMILES: Cc1ccc(NC2CNC2)cc1C(=O)NCc1cccc(-c2ccc(CN3CCCC3)s2)c1] |
| 126 | 246 | 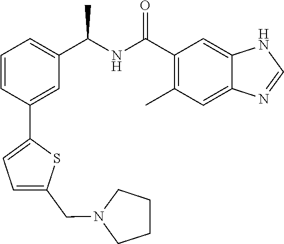 [SMILES: Cc1cc2nc[nH]c2cc1C(=O)N[C@H](C)c1cccc(-c2ccc(CN3CCCC3)s2)c1] |
| 134 | 247 | 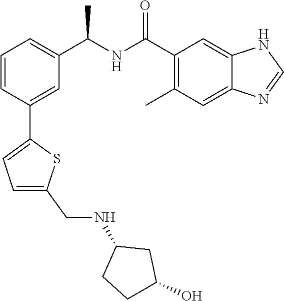 [SMILES: Cc1cc2nc[nH]c2cc1C(=O)N[C@H](C)c1cccc(-c2ccc(CN[C@H]3CC[C@@H](O)C3)s2)c1] |
| 137 | 248 | 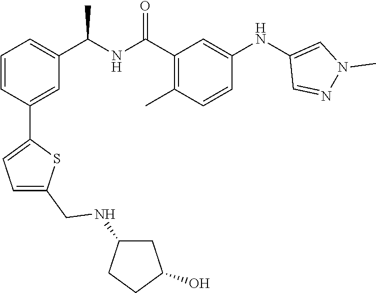 [SMILES: Cc1ccc(Nc2cnn(C)c2)cc1C(=O)N[C@H](C)c1cccc(-c2ccc(CN[C@H]3CC[C@@H](O)C3)s2)c1] |
| 140 | 249 | 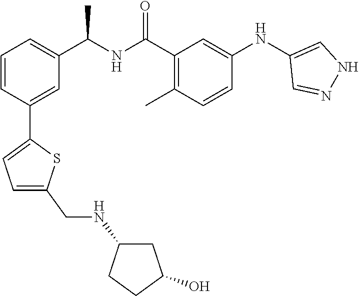 [SMILES: Cc1ccc(Nc2cn[nH]c2)cc1C(=O)N[C@H](C)c1cccc(-c2ccc(CN[C@H]3CC[C@@H](O)C3)s2)c1] |
| 146 | 250 | 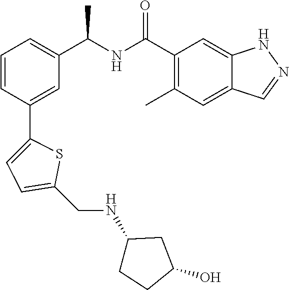 [SMILES: Cc1cc2cn[nH]c2cc1C(=O)N[C@H](C)c1cccc(-c2ccc(CN[C@H]3CC[C@@H](O)C3)s2)c1] |
| 151 | 251 | 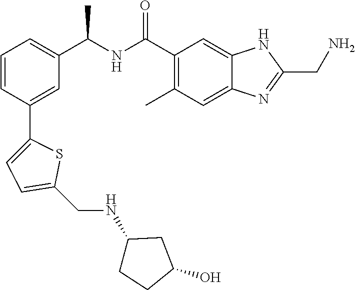 [SMILES: Cc1cc2nc(CN)[nH]c2cc1C(=O)N[C@H](C)c1cccc(-c2ccc(CN[C@H]3CC[C@@H](O)C3)s2)c1] |
| 154 | 252 | 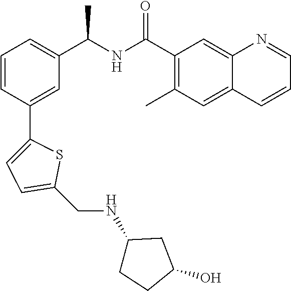 [SMILES: Cc1cc2cccnc2cc1C(=O)N[C@H](C)c1cccc(-c2ccc(CN[C@H]3CC[C@@H](O)C3)s2)c1] |
| 143 | 253 | 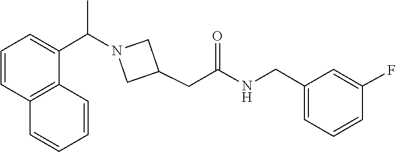 [SMILES: CC(c1cccc2ccccc12)N1CC(CC(=O)NCc2cccc(F)c2)C1] |
| 144 | 254 | 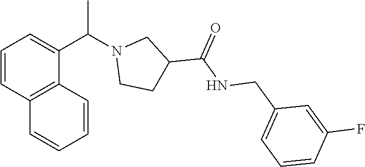 [SMILES: CC(c1cccc2ccccc12)N1CCC(C(=O)NCc2cccc(F)c2)C1] |
| 149 | 255 | 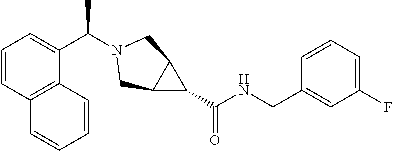 [SMILES: C[C@H](c1cccc2ccccc12)N1C[C@@H]2[C@H](C1)[C@H]2C(=O)NCc1cccc(F)c1] |
| 135 | 256 | 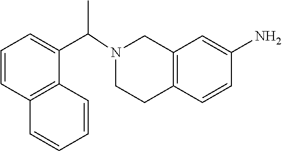 [SMILES: CC(c1cccc2ccccc12)N1CCc2ccc(N)cc2C1] |
| 139 | 257 | 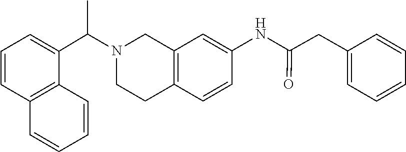 [SMILES: CC(c1cccc2ccccc12)N1CCc2ccc(NC(=O)Cc3ccccc3)cc2C1] |
| 404 | 258 | 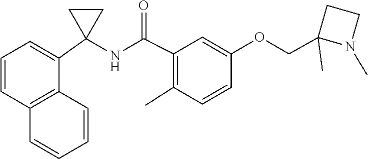 [SMILES: Cc1ccc(OCC2(C)CCN2C)cc1C(=O)NC1(c2cccc3ccccc23)CC1] |
| 406 | 259 | 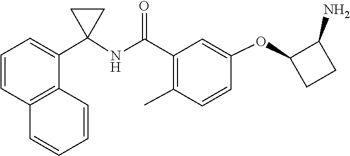 [SMILES: Cc1ccc(O[C@@H]2CC[C@@H]2N)cc1C(=O)NC1(c2cccc3ccccc23)CC1] |
| 416 | 260 | 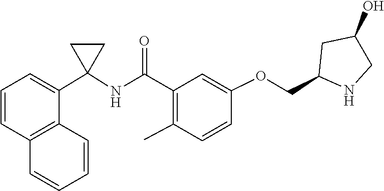 [SMILES: Cc1ccc(OC[C@H]2C[C@@H](O)CN2)cc1C(=O)NC1(c2cccc3ccccc23)CC1] |
| 414 | 261 | 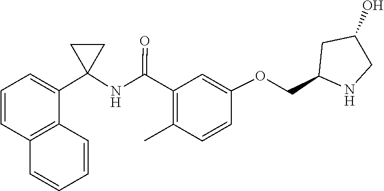 [SMILES: Cc1ccc(OC[C@H]2C[C@H](O)CN2)cc1C(=O)NC1(c2cccc3ccccc23)CC1] |
| 412 | 262 | 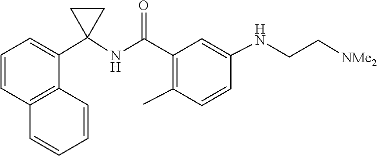 [SMILES: Cc1ccc(NCCN(C)C)cc1C(=O)NC1(c2cccc3ccccc23)CC1] |
| 421 | 263 | 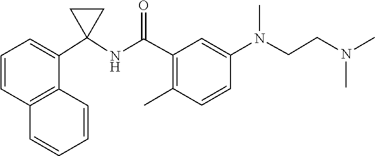 [SMILES: Cc1ccc(N(C)CCN(C)C)cc1C(=O)NC1(c2cccc3ccccc23)CC1] |
| 413 | 264 | 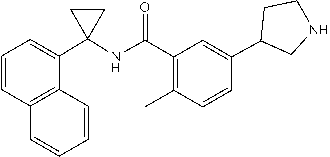 [SMILES: Cc1ccc(C2CCNC2)cc1C(=O)NC1(c2cccc3ccccc23)CC1] |
| 407 | 265 | 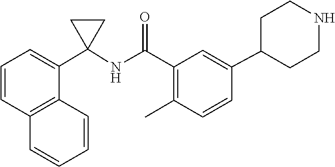 [SMILES: Cc1ccc(C2CCNCC2)cc1C(=O)NC1(c2cccc3ccccc23)CC1] |
| 410 | 266 | 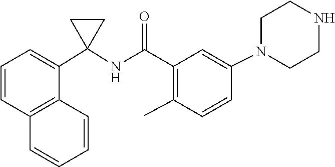 [SMILES: Cc1ccc(N2CCNCC2)cc1C(=O)NC1(c2cccc3ccccc23)CC1] |
| 418 | 267 | 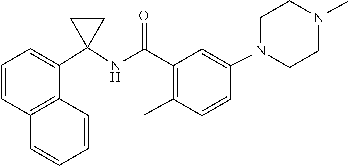 [SMILES: Cc1ccc(N2CCN(C)CC2)cc1C(=O)NC1(c2cccc3ccccc23)CC1] |
| 411 | 268 | 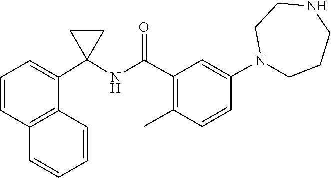 [SMILES: Cc1ccc(N2CCCNCC2)cc1C(=O)NC1(c2cccc3ccccc23)CC1] |
| 405 | 269 | 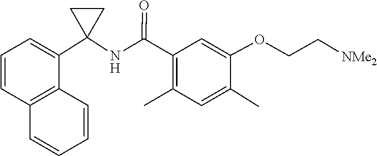 [SMILES: Cc1cc(C)c(C(=O)NC2(c3cccc4ccccc34)CC2)cc1OCCN(C)C] |
| 409 | 270 | 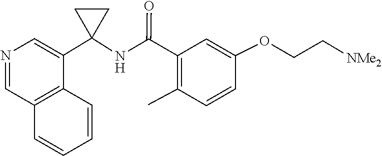 [SMILES: Cc1ccc(OCCN(C)C)cc1C(=O)NC1(c2cncc3ccccc23)CC1] |
| 415 | 271 | 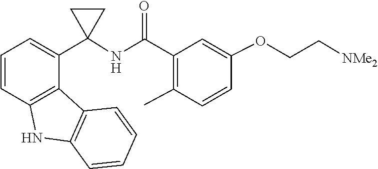 [SMILES: Cc1ccc(OCCN(C)C)cc1C(=O)NC1(c2cccc3[nH]c4ccccc4c23)CC1] |
| 417 | 272 | 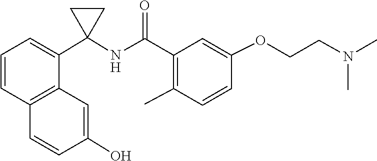 [SMILES: Cc1ccc(OCCN(C)C)cc1C(=O)NC1(c2cccc3ccc(O)cc23)CC1] |
| 408 | 273 | 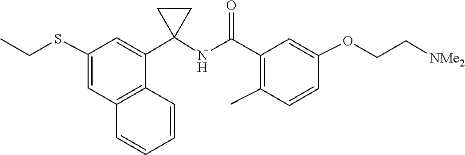 [SMILES: CCSc1cc(C2(NC(=O)c3cc(OCCN(C)C)ccc3C)CC2)c2ccccc2c1] |
| 419 | 274 | 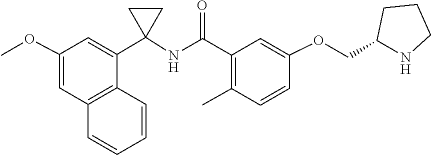 [SMILES: COc1cc(C2(NC(=O)c3cc(OC[C@@H]4CCCN4)ccc3C)CC2)c2ccccc2c1] |
| 420 | 275 | 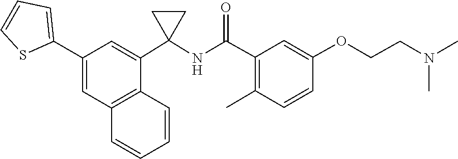 [SMILES: Cc1ccc(OCCN(C)C)cc1C(=O)NC1(c2cc(-c3cccs3)cc3ccccc23)CC1] |
| 459 | 276 | 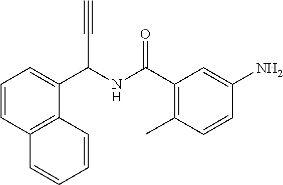 [SMILES: C#CC(NC(=O)c1cc(N)ccc1C)c1cccc2ccccc12] |
| 448 | 277 | 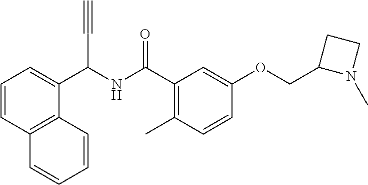 [SMILES: C#CC(NC(=O)c1cc(OCC2CCN2C)ccc1C)c1cccc2ccccc12] |
| 453 | 278 | 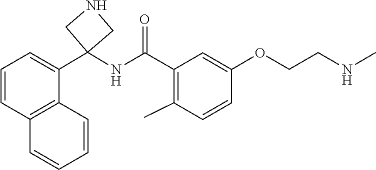 [SMILES: CNCCOc1ccc(C)c(C(=O)NC2(c3cccc4ccccc34)CNC2)c1] |
| 499 | 279 | 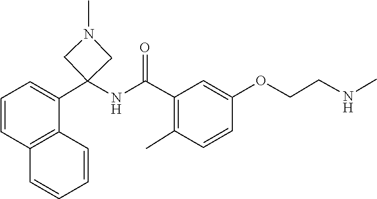 [SMILES: CNCCOc1ccc(C)c(C(=O)NC2(c3cccc4ccccc34)CN(C)C2)c1] |
| 451 | 280 | 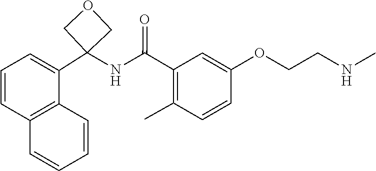 [SMILES: CNCCOc1ccc(C)c(C(=O)NC2(c3cccc4ccccc34)COC2)c1] |
| 450 | 281 | 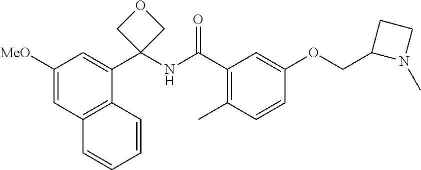 [SMILES: COc1cc(C2(NC(=O)c3cc(OCC4CCN4C)ccc3C)COC2)c2ccccc2c1] |
| 425 | 282 | 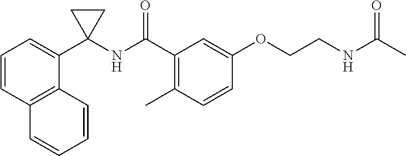 [SMILES: CC(=O)NCCOc1ccc(C)c(C(=O)NC2(c3cccc4ccccc34)CC2)c1] |
| 424 | 283 | 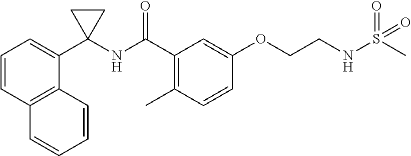 [SMILES: Cc1ccc(OCCNS(C)(=O)=O)cc1C(=O)NC1(c2cccc3ccccc23)CC1] |
| 439 | 284 | 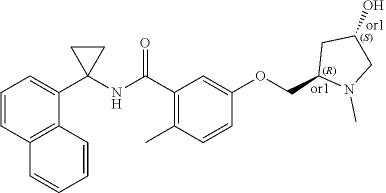 |
| 440 | 285 | 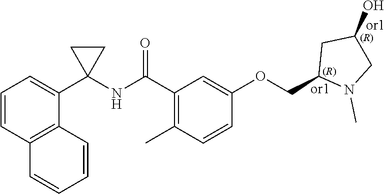 |
| 438 | 286 | 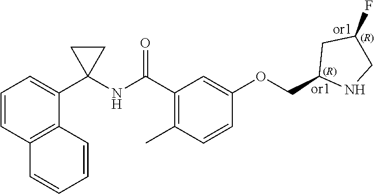 / |
| 437 | 287 | 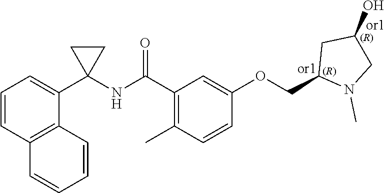 |
| 442 | 288 | 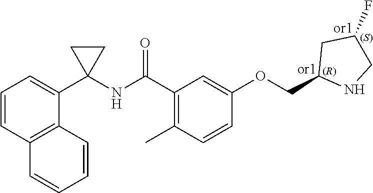 |
| 441 | 289 | 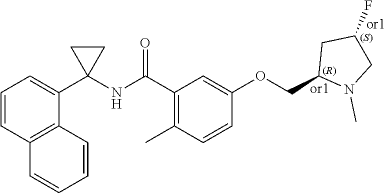 |
| 491 | 290 | 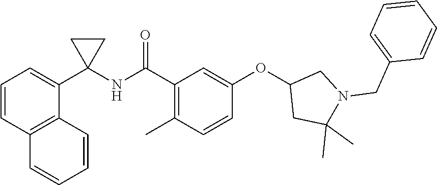 [SMILES: Cc1ccc(OC2CN(Cc3ccccc3)C(C)(C)C2)cc1C(=O)NC1(c2cccc3ccccc23)CC1] |
| 492 | 290 | 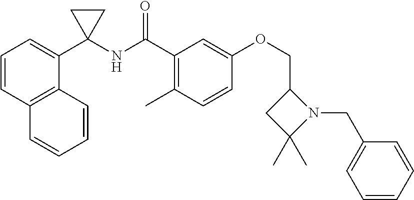 [SMILES: Cc1ccc(OCC2CC(C)(C)N2Cc2ccccc2)cc1C(=O)NC1(c2cccc3ccccc23)CC1] |
| 534 | 291 | 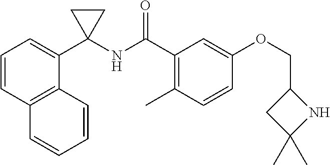 [SMILES: Cc1ccc(OCC2CC(C)(C)N2)cc1C(=O)NC1(c2cccc3ccccc23)CC1] |
| 533 | 292 | 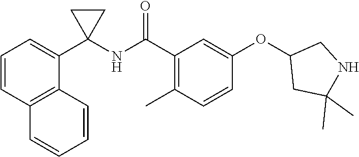 [SMILES: Cc1ccc(OC2CNC(C)(C)C2)cc1C(=O)NC1(c2cccc3ccccc23)CC1] |
| 487 | 293 | 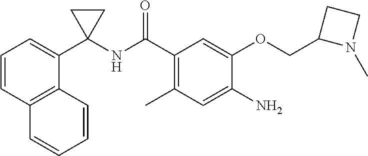 [SMILES: Cc1cc(N)c(OCC2CCN2C)cc1C(=O)NC1(c2cccc3ccccc23)CC1] |
| 435 | 294 | 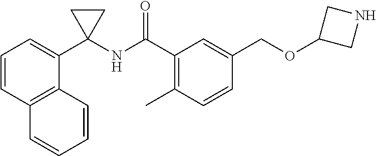 [SMILES: Cc1ccc(COC2CNC2)cc1C(=O)NC1(c2cccc3ccccc23)CC1] |
| 447 | 295 | 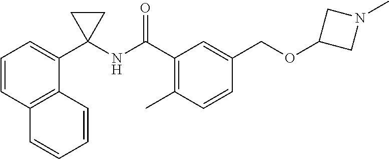 [SMILES: Cc1ccc(COC2CN(C)C2)cc1C(=O)NC1(c2cccc3ccccc23)CC1] |
| 449 | 296 | 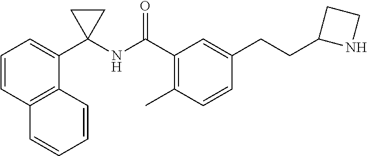 [SMILES: Cc1ccc(CCC2CCN2)cc1C(=O)NC1(c2cccc3ccccc23)CC1] |
| 455 | 297 | 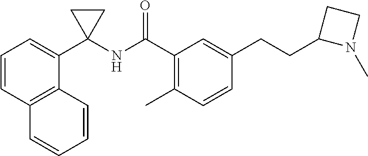 [SMILES: Cc1ccc(CCC2CCN2C)cc1C(=O)NC1(c2cccc3ccccc23)CC1] |
| 431 | 298 | 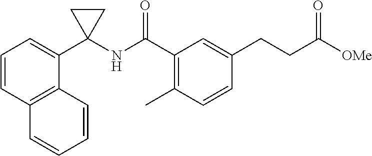 [SMILES: COC(=O)CCc1ccc(C)c(C(=O)NC2(c3cccc4ccccc34)CC2)c1] |
| 452 | 299 | 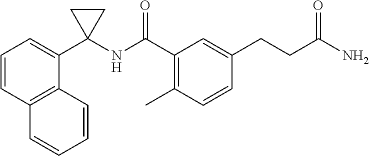 [SMILES: Cc1ccc(CCC(N)=O)cc1C(=O)NC1(c2cccc3ccccc23)CC1] |
| 472 | 300 | 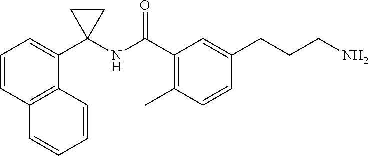 [SMILES: Cc1ccc(CCCN)cc1C(=O)NC1(c2cccc3ccccc23)CC1] |
| 463 | 301 | 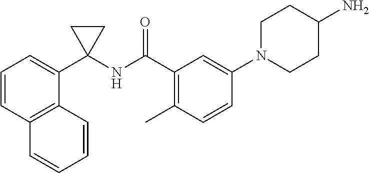 [SMILES: Cc1ccc(N2CCC(N)CC2)cc1C(=O)NC1(c2cccc3ccccc23)CC1] |
| 505 | 302 | 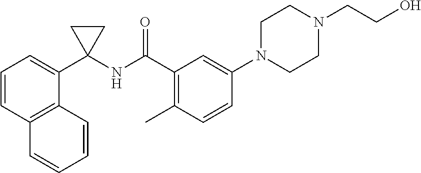 [SMILES: Cc1ccc(N2CCN(CCO)CC2)cc1C(=O)NC1(c2cccc3ccccc23)CC1] |
| 426 | 303 | 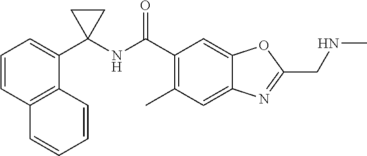 [SMILES: CNCc1nc2cc(C)c(C(=O)NC3(c4cccc5ccccc45)CC3)cc2o1] |
| 432 | 304 | 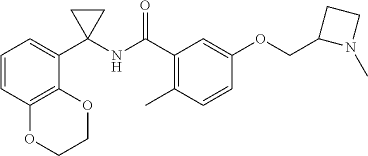 [SMILES: Cc1ccc(OCC2CCN2C)cc1C(=O)NC1(c2cccc3c2OCCO3)CC1] |
| 457 | 305 | 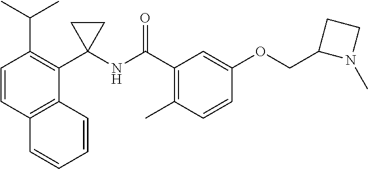 [SMILES: Cc1ccc(OCC2CCN2C)cc1C(=O)NC1(c2c(C(C)C)ccc3ccccc23)CC1] |
| 445 | 306 | 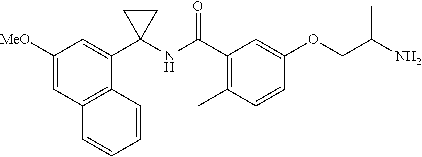 [SMILES: COc1cc(C2(NC(=O)c3cc(OCC(C)N)ccc3C)CC2)c2ccccc2c1] |
| 490 | 307 | 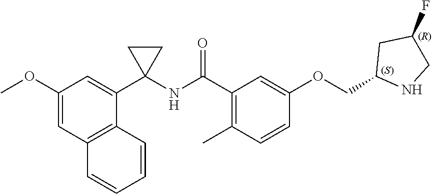 |
| 489 | 308 | 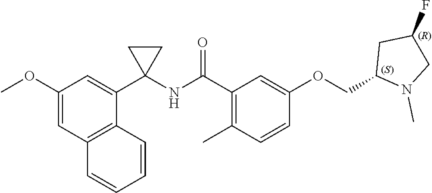 |
| 433 | 309 | 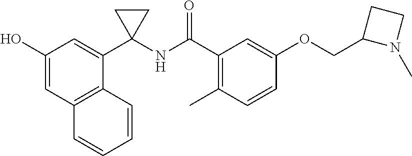 [SMILES: Cc1ccc(OCC2CCN2C)cc1C(=O)NC1(c2cc(O)cc3ccccc23)CC1] |
| 474 | 310 | 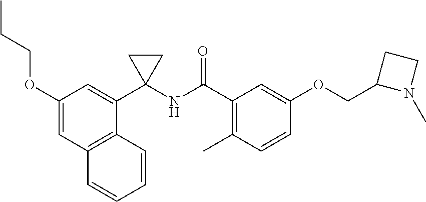 [SMILES: CCCOc1cc(C2(NC(=O)c3cc(OCC4CCN4C)ccc3C)CC2)c2ccccc2c1] |
| 461 | 311 | 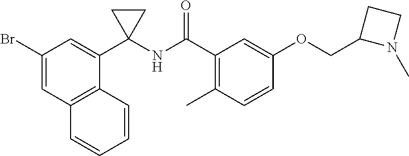 [SMILES: Cc1ccc(OCC2CCN2C)cc1C(=O)NC1(c2cc(Br)cc3ccccc23)CC1] |
| 541 | 312 | 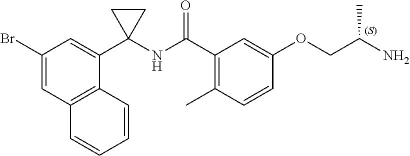 |
| 443 | 313 | 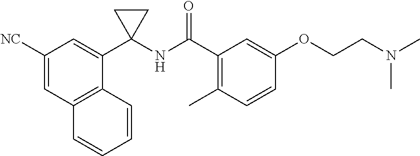 [SMILES: Cc1ccc(OCCN(C)C)cc1C(=O)NC1(c2cc(C#N)cc3ccccc23)CC1] |
| 484 | 314 | 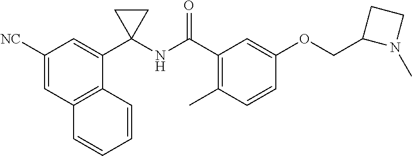 [SMILES: Cc1ccc(OCC2CCN2C)cc1C(=O)NC1(c2cc(C#N)cc3ccccc23)CC1] |
| 482 | 315 | 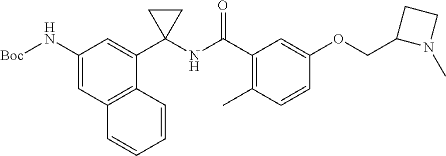 [SMILES: Cc1ccc(OCC2CCN2C)cc1C(=O)NC1(c2cc(NC(=O)OC(C)(C)C)cc3ccccc23)CC1] |
| 496 | 316 | 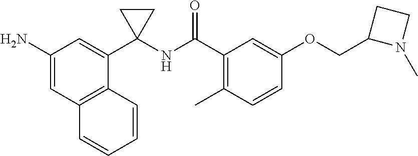 [SMILES: Cc1ccc(OCC2CCN2C)cc1C(=O)NC1(c2cc(N)cc3ccccc23)CC1] |
| 495 | 317 | 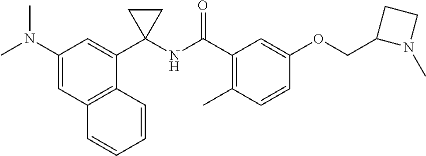 |
| 520 | 318 | 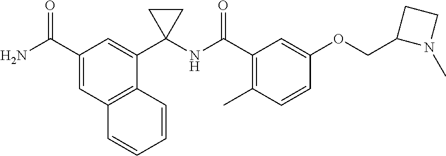 [SMILES: Cc1ccc(OCC2CCN2C)cc1C(=O)NC1(c2cc(C(N)=O)cc3ccccc23)CC1] |
| 481 | 319 | 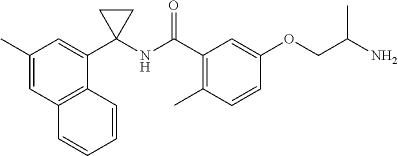 [SMILES: Cc1cc(C2(NC(=O)c3cc(OCC(C)N)ccc3C)CC2)c2ccccc2c1] |
| 501 | 320 | 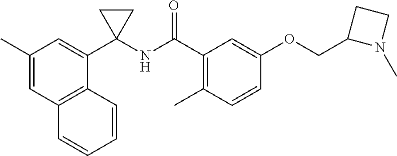 |
| 508 | 321 | 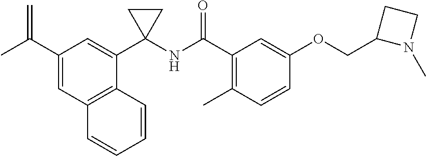 [SMILES: C=C(C)c1cc(C2(NC(=O)c3cc(OCC4CCN4C)ccc3C)CC2)c2ccccc2c1] |
| 521 | 322 | 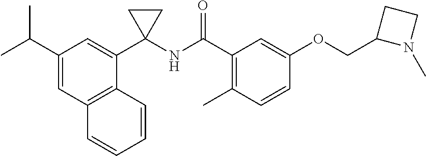 [SMILES: Cc1ccc(OCC2CCN2C)cc1C(=O)NC1(c2cc(C(C)C)cc3ccccc23)CC1] |
| 423 | 323 | 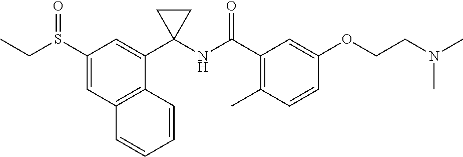 [SMILES: CCS(=O)c1cc(C2(NC(=O)c3cc(OCCN(C)C)ccc3C)CC2)c2ccccc2c1] |
| 471 | 324 | 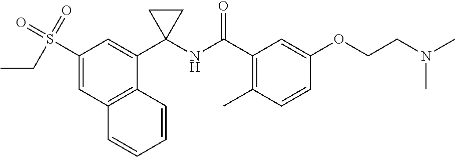 [SMILES: CCS(=O)(=O)c1cc(C2(NC(=O)c3cc(OCCN(C)C)ccc3C)CC2)c2ccccc2c1] |
| 486 | 325 | 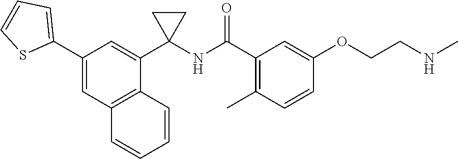 [SMILES: CNCCOc1ccc(C)c(C(=O)NC2(c3cc(-c4cccs4)cc4ccccc34)CC2)c1] |
| 539 | 326 | 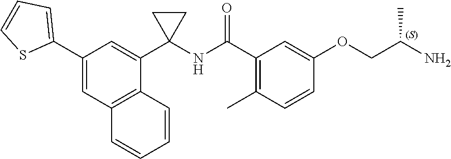 |
| 468 | 327 | 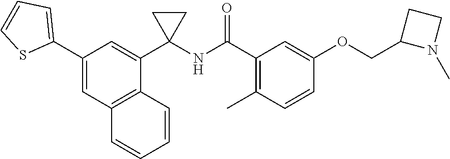 [SMILES: Cc1ccc(OCC2CCN2C)cc1C(=O)NC1(c2cc(-c3cccs3)cc3ccccc23)CC1] |
| 470 | 328 | 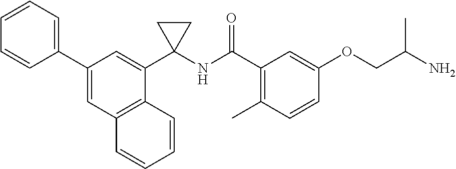 [SMILES: Cc1ccc(OCC(C)N)cc1C(=O)NC1(c2cc(-c3ccccc3)cc3ccccc23)CC1] |
| 475 | 329 | 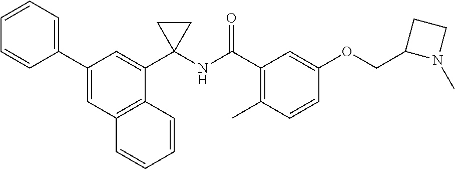 [SMILES: Cc1ccc(OCC2CCN2C)cc1C(=O)NC1(c2cc(-c3ccccc3)cc3ccccc23)CC1] |
| 477 | 330 | 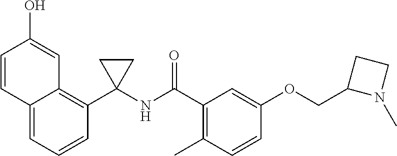 [SMILES: Cc1ccc(OCC2CCN2C)cc1C(=O)NC1(c2cccc3ccc(O)cc23)CC1] |
| 465 | 331 | 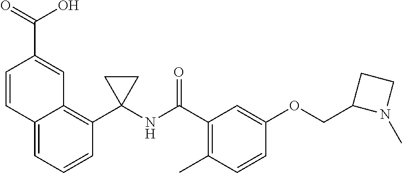 [SMILES: Cc1ccc(OCC2CCN2C)cc1C(=O)NC1(c2cccc3ccc(C(=O)O)cc23)CC1] |
| 483 | 332 | 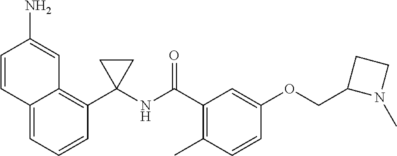 [SMILES: Cc1ccc(OCC2CCN2C)cc1C(=O)NC1(c2cccc3ccc(N)cc23)CC1] |
| 434 | 333 | 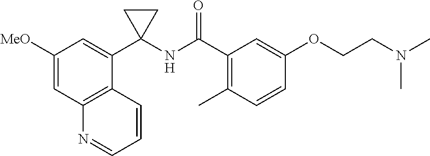 [SMILES: COc1cc(C2(NC(=O)c3cc(OCCN(C)C)ccc3C)CC2)c2cccnc2c1] |
| 530 | 334 | 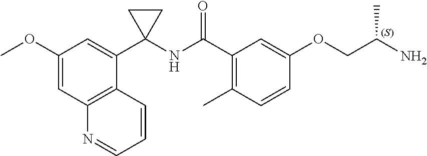 |
| 526 | 335 | 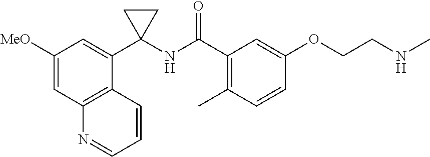 [SMILES: CNCCOc1ccc(C)c(C(=O)NC2(c3cc(OC)cc4ncccc34)CC2)c1] |
| 560 | 336 | 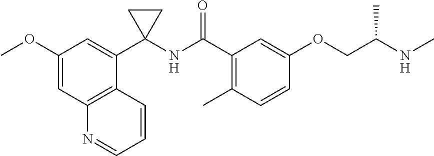 |
| 575 | 337 | 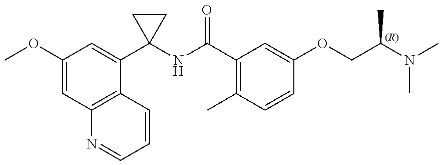 |
| 511 | 338 | 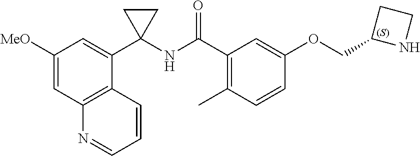 |
| 527 | 339 | 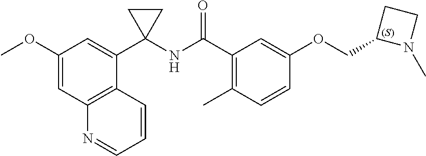 |
| 462 | 340 | 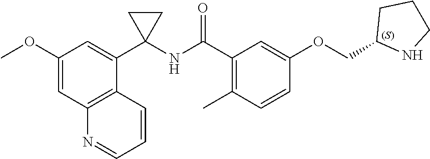 |
| 528 | 341 | 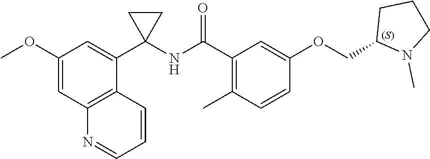 |
| 550 | 342 | 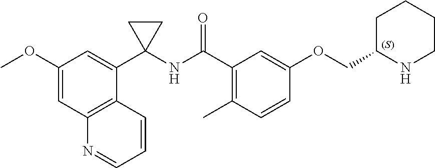 |
| 561 | 343 | 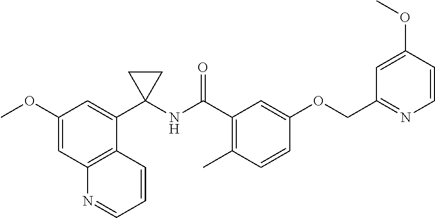 [SMILES: COc1ccnc(COc2ccc(C)c(C(=O)NC3(c4cc(OC)cc5ncccc45)CC3)c2)c1] |
| 584 | 344 | 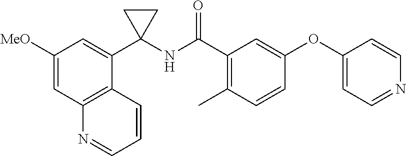 [SMILES: COc1cc(C2(NC(=O)c3cc(Oc4ccncc4)ccc3C)CC2)c2cccnc2c1] |
| 597 | 345 | 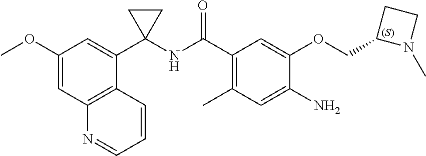 |
| 587 | 346 | 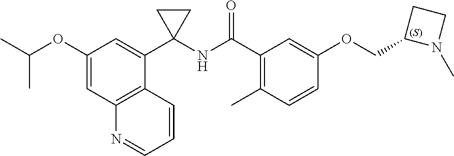 |
| 598 | 347 | 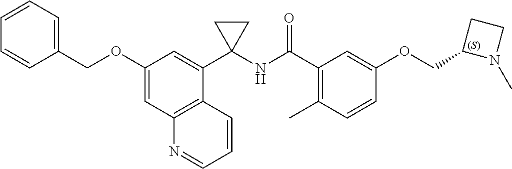 |
| 563 | 348 | 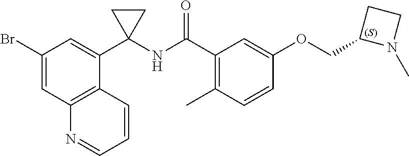 |
| 497 | 349 | 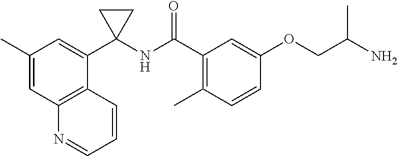 [SMILES: Cc1cc(C2(NC(=O)c3cc(OCC(C)N)ccc3C)CC2)c2cccnc2c1] |
| 581 | 350 | 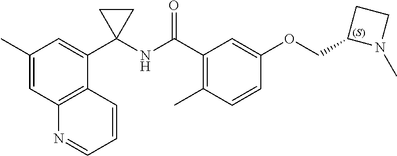 |
| 600 | 351 | 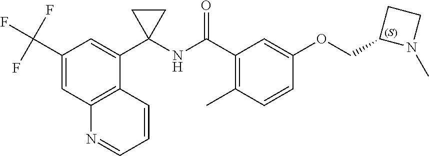 |
| 568 | 352 | 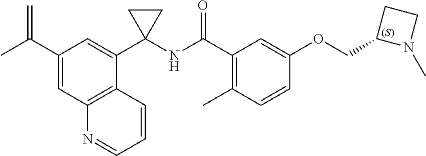 |
| 551 | 353 | 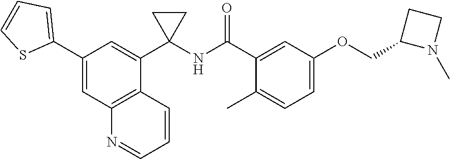 [SMILES: Cc1ccc(OC[C@@H]2CCN2C)cc1C(=O)NC1(c2cc(-c3cccs3)cc3ncccc23)CC1] |
| 588 | 354 | 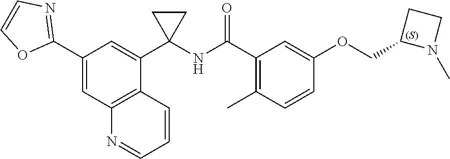 |
| 436 | 355 | 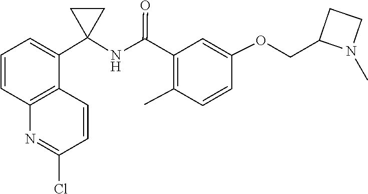 [SMILES: Cc1ccc(OCC2CCN2C)cc1C(=O)NC1(c2cccc3nc(Cl)ccc23)CC1] |
| 529 | 356 | 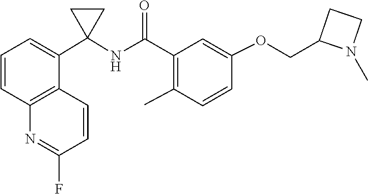 [SMILES: Cc1ccc(OCC2CCN2C)cc1C(=O)NC1(c2cccc3nc(F)ccc23)CC1] |
| 547 | 357 | 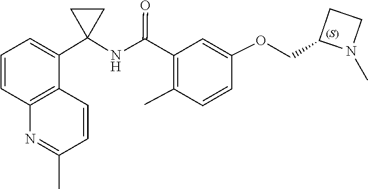 |
| 545 | 358 | 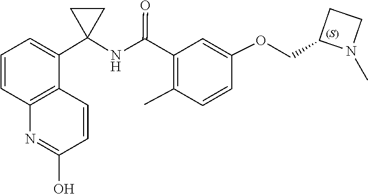 |
| 544 | 359 | 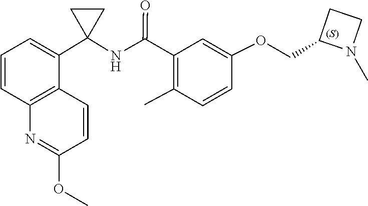 |
| 548 | 360 | 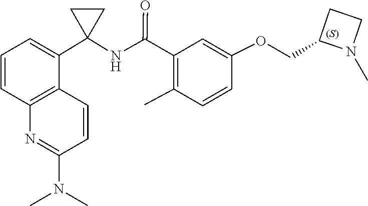 |
| 555 | 361 | 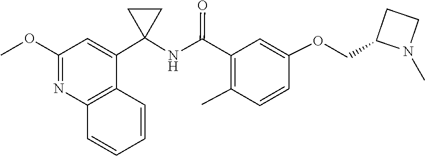 |
| 467 | 362 | 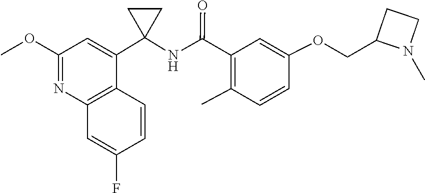 |
| 565 | 363 | 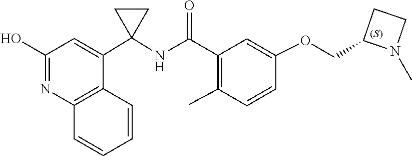 |
| 542 | 364 | 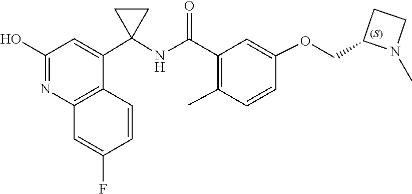 |
| 586 | 365 | 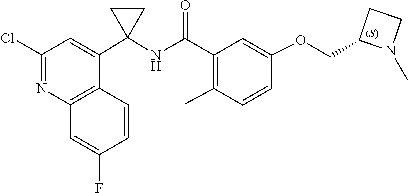 |
| 538 | 366 | 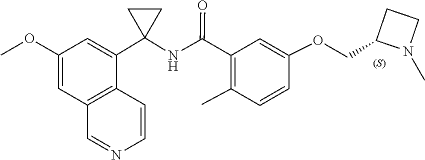 |
| 509 | 367 | 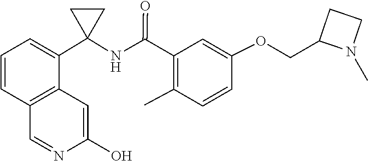 [SMILES: Cc1ccc(OCC2CCN2C)cc1C(=O)NC1(c2cccc3cnc(O)cc23)CC1] |
| 549 | 368 | 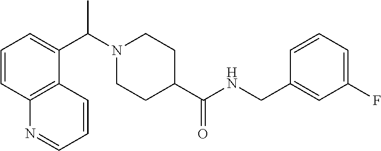 [SMILES: CC(c1cccc2ncccc12)N1CCC(C(=O)NCc2cccc(F)c2)CC1] |
| 522 | 369 | 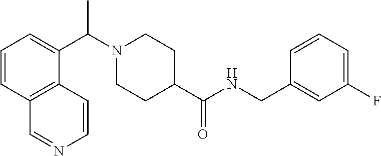 [SMILES: CC(c1cccc2cnccc12)N1CCC(C(=O)NCc2cccc(F)c2)CC1] |
| 524 | 370 | 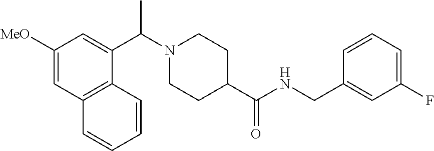 [SMILES: COc1cc(C(C)N2CCC(C(=O)NCc3cccc(F)c3)CC2)c2ccccc2c1] |
| 106 | 371 | 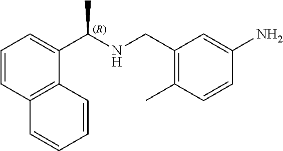 |
| 178 | 372 | 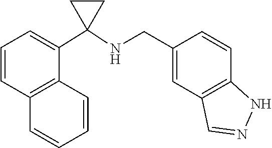 [SMILES: c1ccc2c(C3(NCc4ccc5[nH]ncc5c4)CC3)cccc2c1] |
| 184 | 373 | 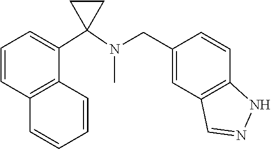 [SMILES: CN(Cc1ccc2[nH]ncc2c1)C1(c2cccc3ccccc23)CC1] |
| 208 | 374 | 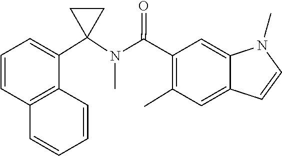 [SMILES: Cc1cc2ccn(C)c2cc1C(=O)N(C)C1(c2cccc3ccccc23)CC1] |
| 179 | 375 | 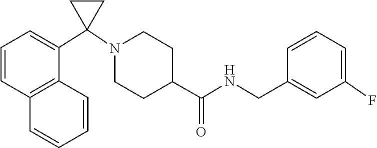 [SMILES: O=C(NCc1cccc(F)c1)C1CCN(C2(c3cccc4ccccc34)CC2)CC1] |
| 577 | 376 | 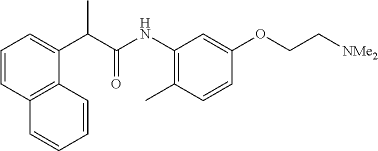 [SMILES: Cc1ccc(OCCN(C)C)cc1NC(=O)C(C)c1cccc2ccccc12] |
| 580 | 377 | 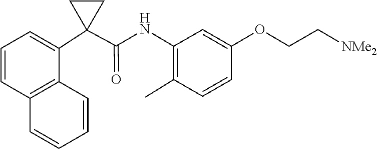 [SMILES: Cc1ccc(OCCN(C)C)cc1NC(=O)C1(c2cccc3ccccc23)CC1] |
| 111 | 378 | 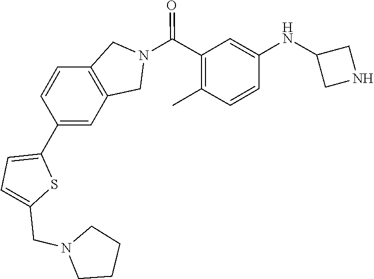 [SMILES: Cc1ccc(NC2CNC2)cc1C(=O)N1Cc2ccc(-c3ccc(CN4CCCC4)s3)cc2C1] |
| 116 | 379 | 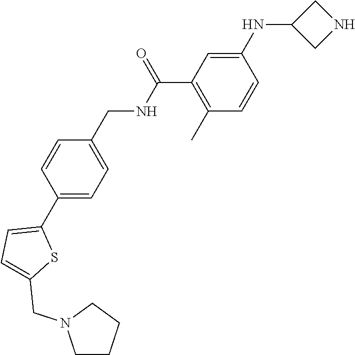 [SMILES: Cc1ccc(NC2CNC2)cc1C(=O)NCc1ccc(-c2ccc(CN3CCCC3)s2)cc1] |
| 161 | 380 | 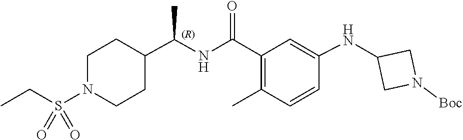 |
| 166 | 381 | 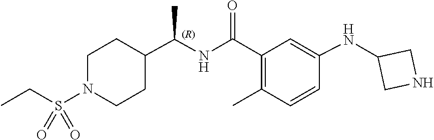 |
| 108 | 382 | 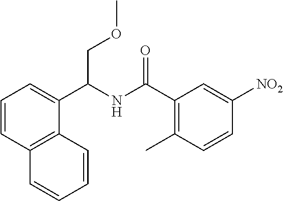 [SMILES: COCC(NC(=O)c1cc([N+](=O)[O-])ccc1C)c1cccc2ccccc12] |
| 118 | 383 | 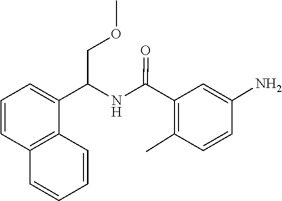 [SMILES: COCC(NC(=O)c1cc(N)ccc1C)c1cccc2ccccc12] |
| 105 | 384 | 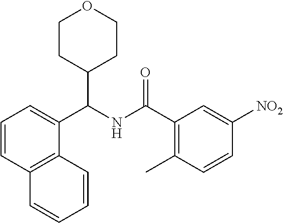 [SMILES: Cc1ccc([N+](=O)[O-])cc1C(=O)NC(c1cccc2ccccc12)C1CCOCC1] |
| 109 | 385 | 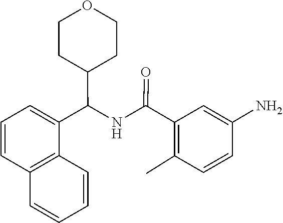 [SMILES: Cc1ccc(N)cc1C(=O)NC(c1cccc2ccccc12)C1CCOCC1] |
| 242 | 386 | 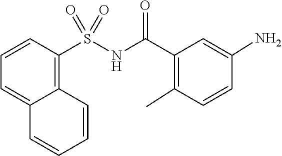 [SMILES: Cc1ccc(N)cc1C(=O)NS(=O)(=O)c1cccc2ccccc12] |
| 138 | 387 | 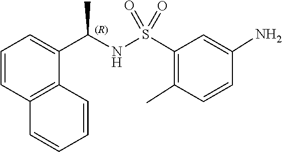 |
| 217 | 388 | 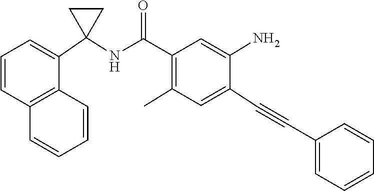 [SMILES: Cc1cc(C#Cc2ccccc2)c(N)cc1C(=O)NC1(c2cccc3ccccc23)CC1] |
| 624 | 389 | 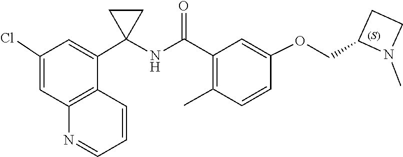 |
| 729 | 390 | 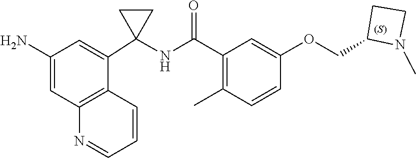 |
| 627 | 391 | 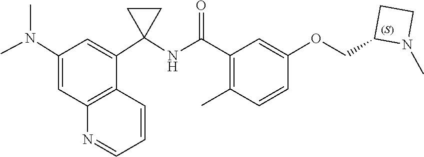 |
| 611 | 392 | 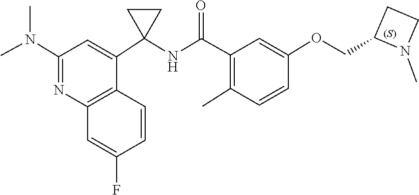 |
| 743 | 393 | 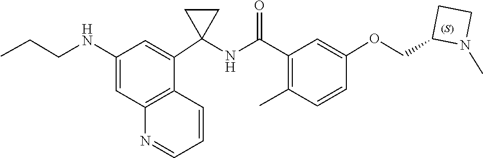 |
| 744 | 394 | 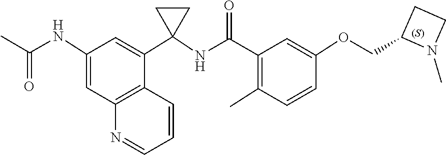 |
| 786 | 395 | 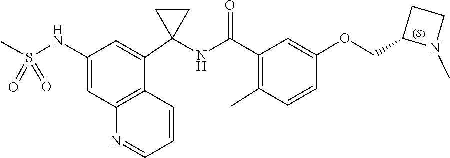 |
| 757 | 396 | 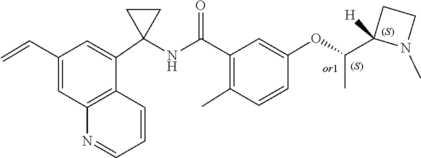 |
| 713 | 397 | 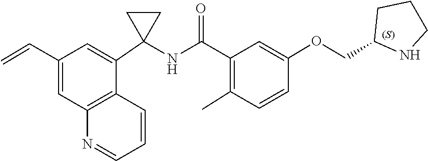 |
| 748 | 398 | 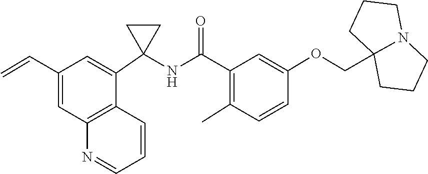 [SMILES: C=Cc1cc(C2(NC(=O)c3cc(OCC45CCCN4CCC5)ccc3C)CC2)c2cccnc2c1] |
| 749 | 399 | 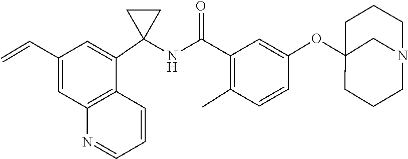 [SMILES: C=Cc1cc(C2(NC(=O)c3cc(OC45CCCN(CCC4)C5)ccc3C)CC2)c2cccnc2c1] |
| 800 | 400 | 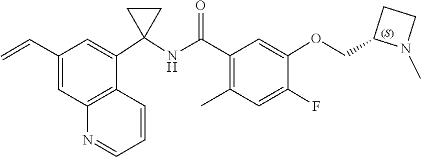 |
| 726 | 401 | 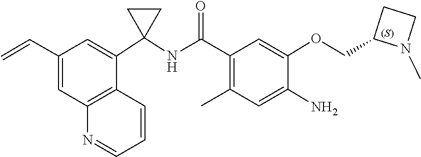 |
| 746 | 402 | 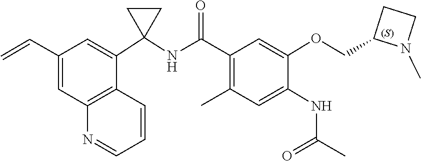 |
| 785 | 403 | 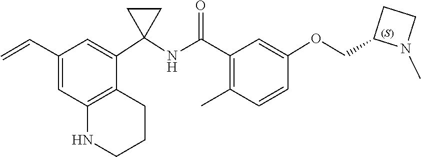 |
| 796 | 404 | 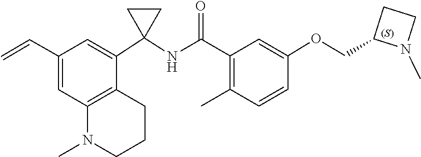 |
| 660 | 405 | 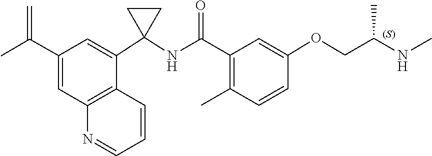 |
| 638 | 406 | 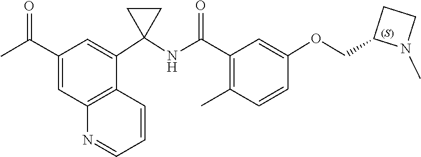 |
| 676 | 407 | 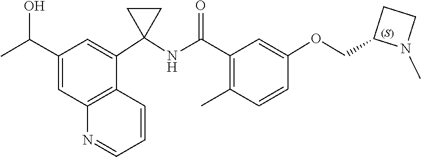 |
| 697 | 408 | 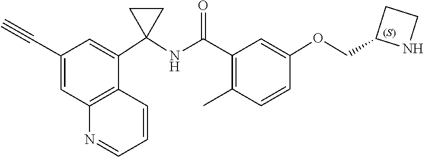 |
| 620 | 409 | 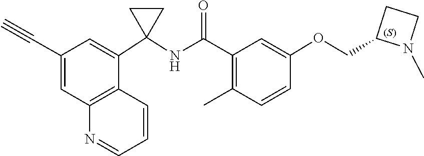 |
| 696 | 410 | 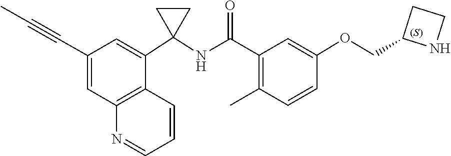 |
| 803 | 411 | 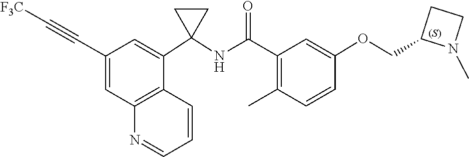 |
| 738 | 412 | 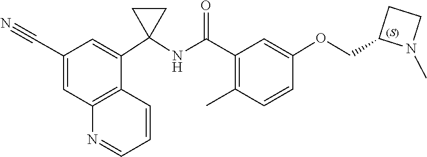 |
| 677 | 413 | 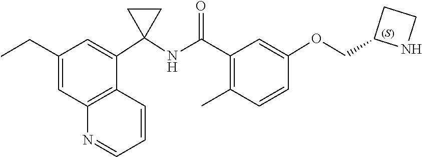 |
| 692 | 414 | 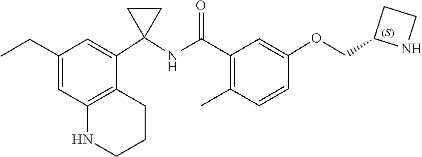 |
| 761 | 415 | 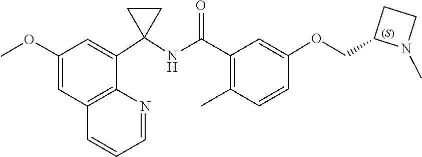 |
| 626 | 416 | 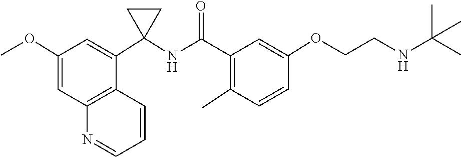 [SMILES: COc1cc(C2(NC(=O)c3cc(OCCNC(C)(C)C)ccc3C)CC2)c2cccnc2c1] |
| 797 | 417 | 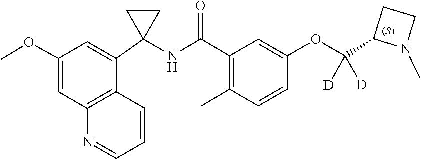 |
| 732 | 418 | 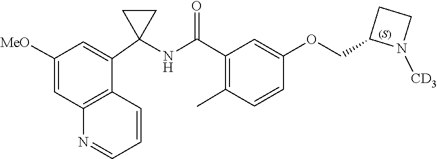 |
| 775 | 419 | 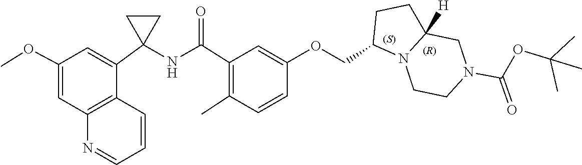 |
| 776 | 419 | 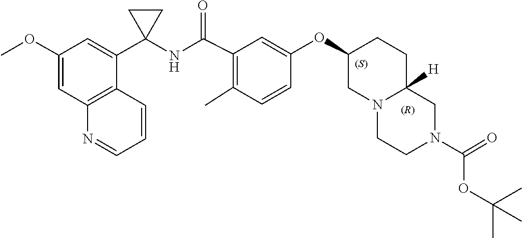 |
| 789 | 420 | 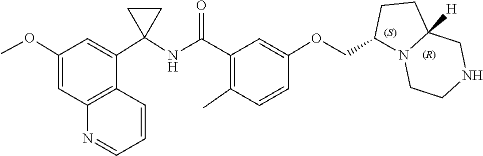 |
| 790 | 420 | 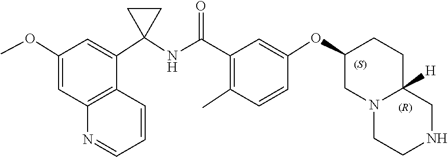 |
| 783 | 421 | 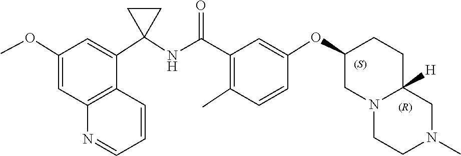 |
| 784 | 421 | 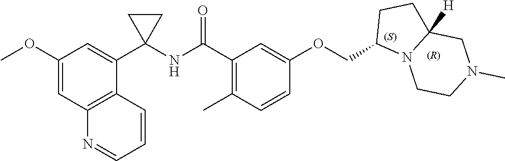 |
| 799 | 422 | 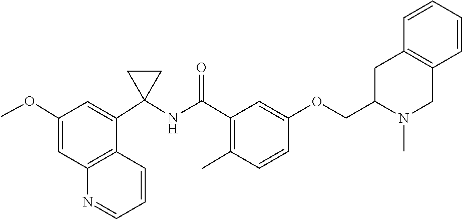 [SMILES: COc1cc(C2(NC(=O)c3cc(OCC4Cc5ccccc5CN4C)ccc3C)CC2)c2cccnc2c1] |
| 794 | 423 | 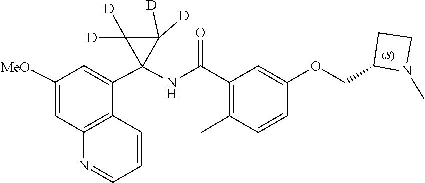 |
| 750 | 424 | 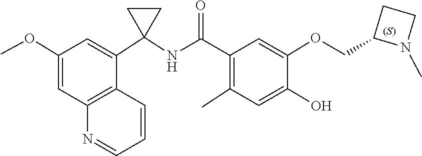 |
| 737 | 425 | 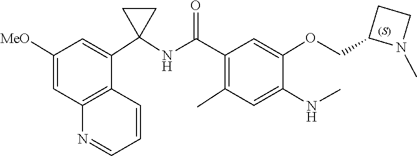 |
| 625 | 426 | 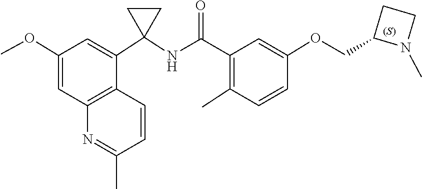 |
| 791 | 427 | 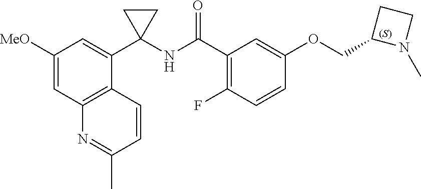 |
| 817 | 428 | 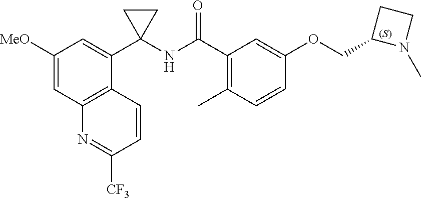 |
| 809 | 429 | 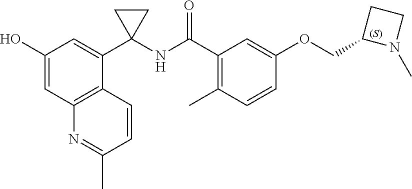 |
| 795 | 430 | 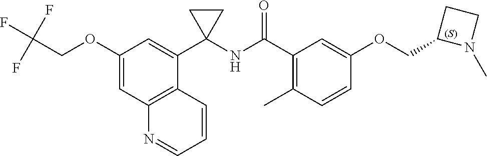 |
| 815 | 431 | 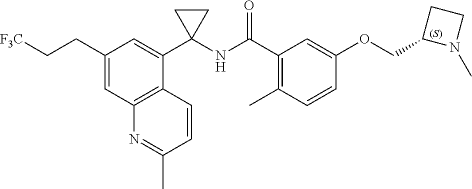 |
| 648 | 432 | 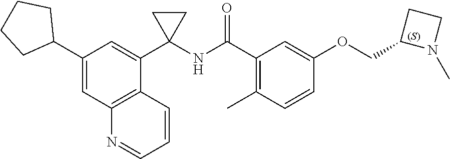 |
| 683 | 433 | 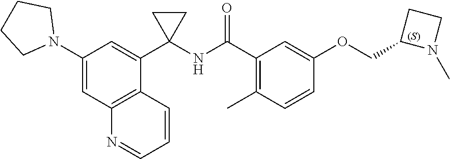 |
| 630 | 434 | 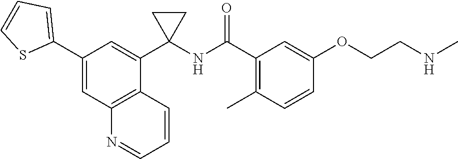 [SMILES: CNCCOc1ccc(C)c(C(=O)NC2(c3cc(-c4cccs4)cc4ncccc34)CC2)c1] |
| 608 | 435 | 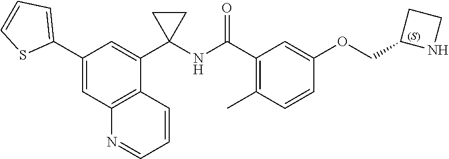 |
| 753 | 436 | 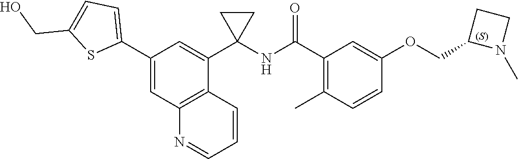 |
| 780 | 437 | 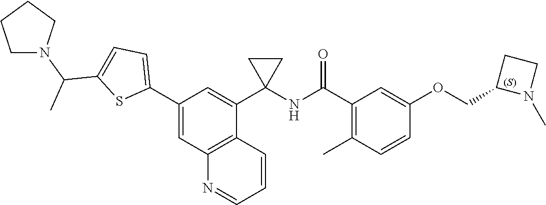 |
| 669 | 438 | 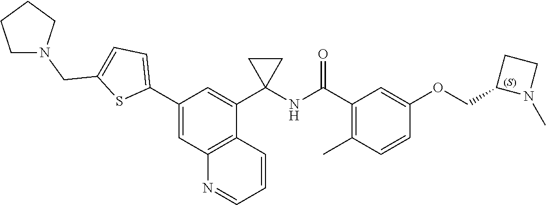 |
| 767 | 439 | 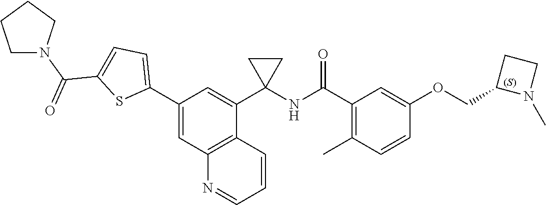 |
| 812 | 440 | 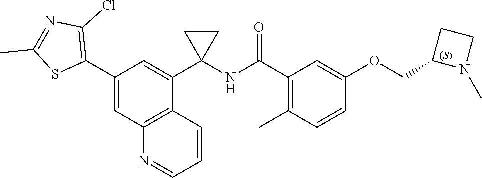 |
| 814 | 441 | 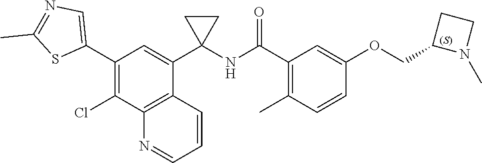 |
| 782 | 442 | 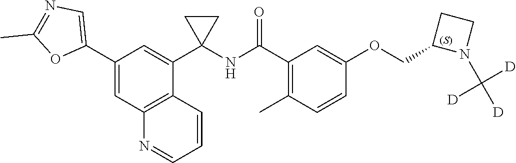 |
| 806 | 443 | 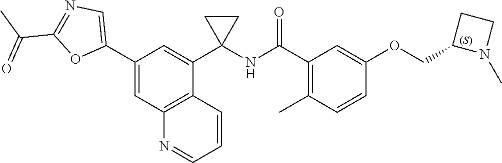 |
| 619 | 444 | 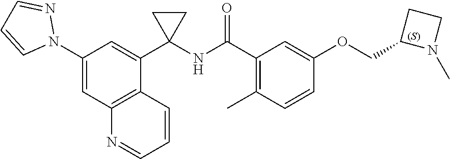 |
| 653 | 445 | 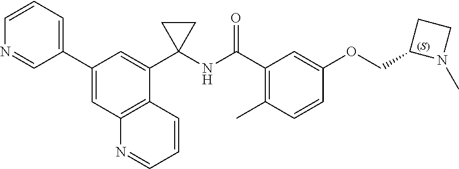 |
| 689 | 446 | 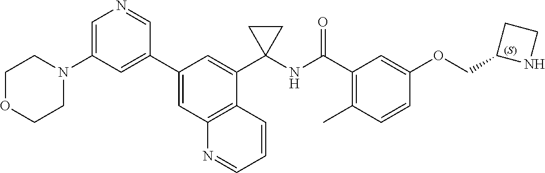 |
| 821 | 447 | 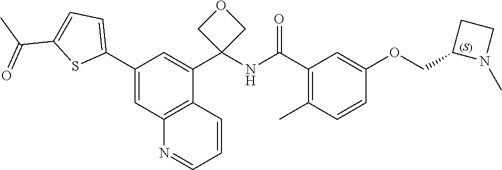 |
| 808 | 448 | 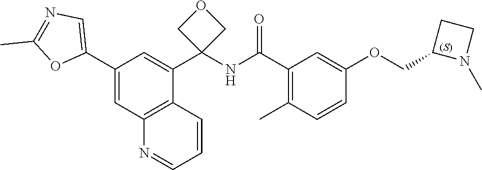 |
| 181 | 449 | 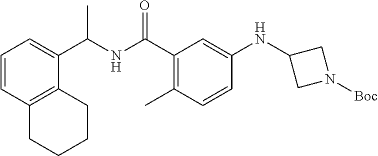 [SMILES: Cc1ccc(NC2CN(C(=O)OC(C)(C)C)C2)cc1C(=O)NC(C)c1cccc2c1CCCC2] |
| 104 | 450 | 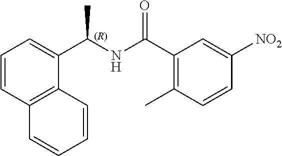 |
| 188 | 451 | 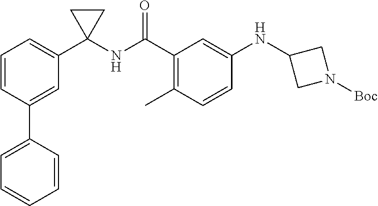 [SMILES: Cc1ccc(NC2CN(C(=O)OC(C)(C)C)C2)cc1C(=O)NC1(c2cccc(-c3ccccc3)c2)CC1] |
| 127 | 452 | 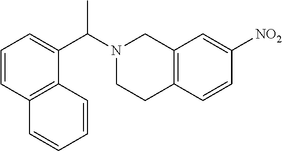 [SMILES: CC(c1cccc2ccccc12)N1CCc2ccc([N+](=O)[O-])cc2C1] |
| 739 | 453 | 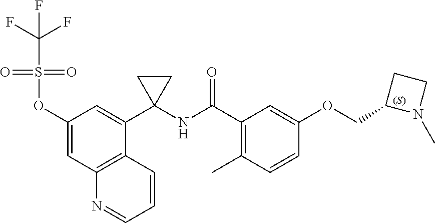 |
| 110 | 454 | 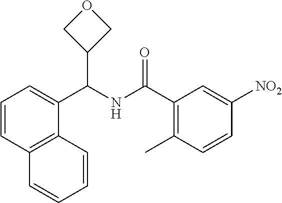 [SMILES: Cc1ccc([N+](=O)[O-])cc1C(=O)NC(c1cccc2ccccc12)C1COC1] |
| 123 | 455 | 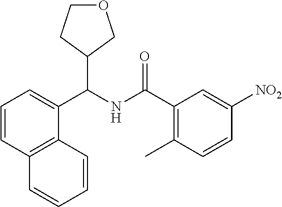 [SMILES: Cc1ccc([N+](=O)[O-])cc1C(=O)NC(c1cccc2ccccc12)C1CCOC1] |
| 113 | 456 | 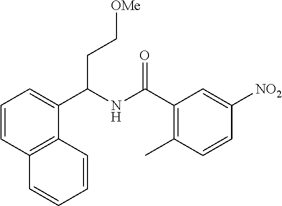 [SMILES: COCCC(NC(=O)c1cc([N+](=O)[O-])ccc1C)c1cccc2ccccc12] |
| 114 | 457 | 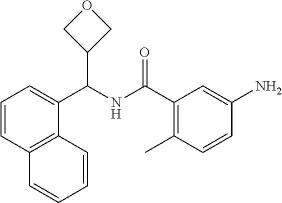 [SMILES: Cc1ccc(N)cc1C(=O)NC(c1cccc2ccccc12)C1COC1] |
| 117 | 458 | 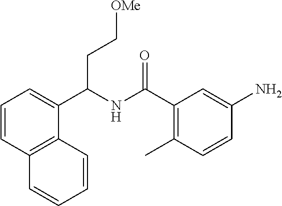 [SMILES: COCCC(NC(=O)c1cc(N)ccc1C)c1cccc2ccccc12] |
| 182 | 459 | 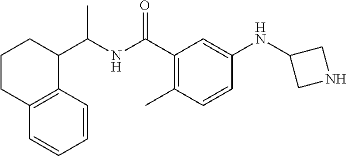 [SMILES: Cc1ccc(NC2CNC2)cc1C(=O)NC(C)C1CCCc2ccccc21] |
| 607 | 460 | 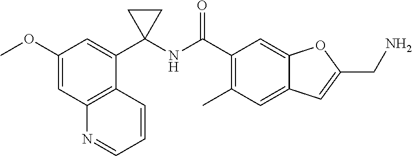 [SMILES: COc1cc(C2(NC(=O)c3cc4oc(CN)cc4cc3C)CC2)c2cccnc2c1] |
| 606 | 461 | 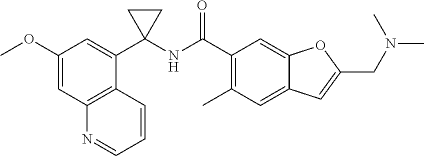 |
| 513 | 462 | 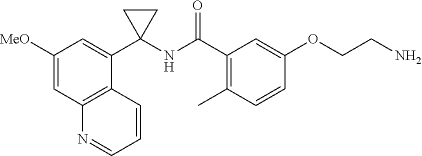 [SMILES: COc1cc(C2(NC(=O)c3cc(OCCN)ccc3C)CC2)c2cccnc2c1] |
| 430 | 463 | 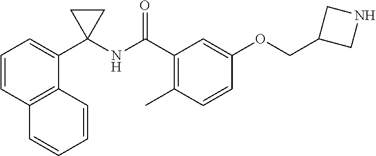 [SMILES: Cc1ccc(OCC2CNC2)cc1C(=O)NC1(c2cccc3ccccc23)CC1] |
| 428 | 464 | 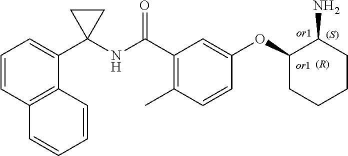 |
| 429 | 465 | 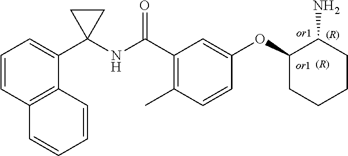 |
| 277 | 466 | 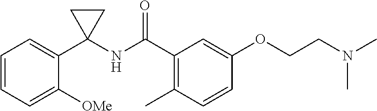 [SMILES: COc1ccccc1C1(NC(=O)c2cc(OCCN(C)C)ccc2C)CC1] |
| 278 | 467 | 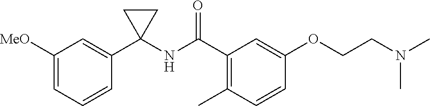 [SMILES: COc1cccc(C2(NC(=O)c3cc(OCCN(C)C)ccc3C)CC2)c1] |
| 569 | 468 | 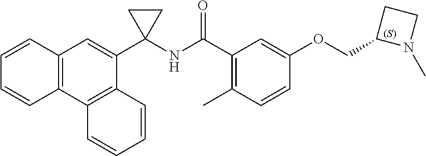 |
| 427 | 469 | 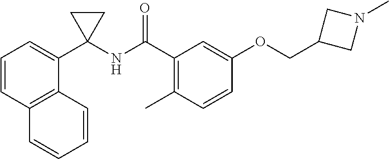 [SMILES: Cc1ccc(OCC2CN(C)C2)cc1C(=O)NC1(c2cccc3ccccc23)CC1] |
| 306 | 470 | 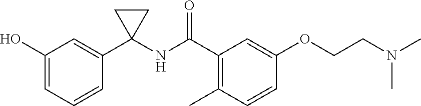 [SMILES: Cc1ccc(OCCN(C)C)cc1C(=O)NC1(c2cccc(O)c2)CC1] |
| 572 | 471 | 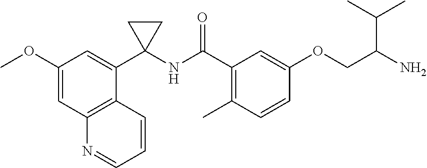 [SMILES: COc1cc(C2(NC(=O)c3cc(OCC(N)C(C)C)ccc3C)CC2)c2cccnc2c1] |
| 567 | 472 | 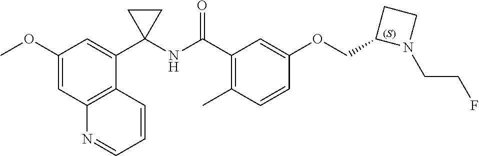 |
| 571 | 473 | 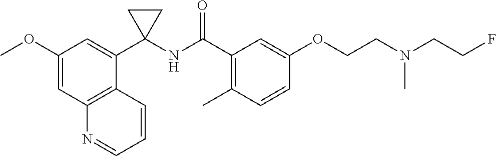 [SMILES: COc1cc(C2(NC(=O)c3cc(OCCN(C)CCF)ccc3C)CC2)c2cccnc2c1] |
| 444 | 474 | 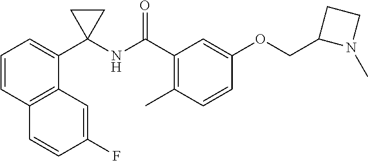 [SMILES: Cc1ccc(OCC2CCN2C)cc1C(=O)NC1(c2cccc3ccc(F)cc23)CC1] |
| 559 | 475 | 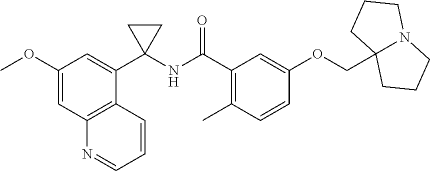 [SMILES: COc1cc(C2(NC(=O)c3cc(OCC45CCCN4CCC5)ccc3C)CC2)c2cccnc2c1] |
| 596 | 476 | 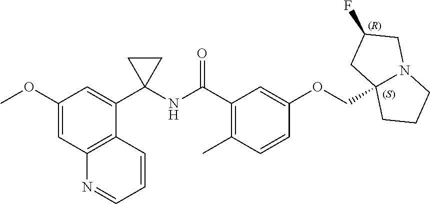 |
| 454 | 477 | 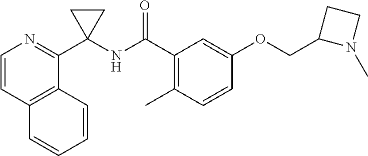 [SMILES: Cc1ccc(OCC2CCN2C)cc1C(=O)NC1(c2nccc3ccccc23)CC1] |
| 473 | 478 | 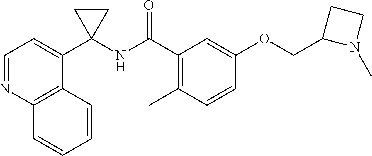 [SMILES: Cc1ccc(OCC2CCN2C)cc1C(=O)NC1(c2ccnc3ccccc23)CC1] |
| 456 | 479 | 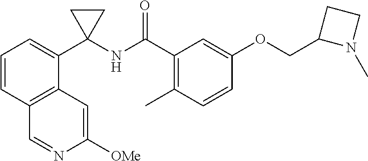 [SMILES: COc1cc2c(C3(NC(=O)c4cc(OCC5CCN5C)ccc4C)CC3)cccc2cn1] |
| 458 | 480 | 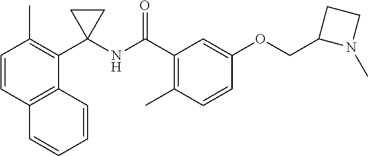 [SMILES: Cc1ccc(OCC2CCN2C)cc1C(=O)NC1(c2c(C)ccc3ccccc23)CC1] |
| 507 | 481 | 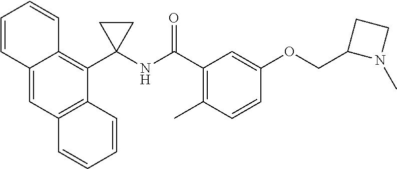 [SMILES: Cc1ccc(OCC2CCN2C)cc1C(=O)NC1(c2c3ccccc3cc3ccccc23)CC1] |
| 488 | 482 | 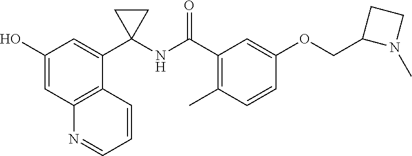 [SMILES: Cc1ccc(OCC2CCN2C)cc1C(=O)NC1(c2cc(O)cc3ncccc23)CC1] |
| 617 | 483 | 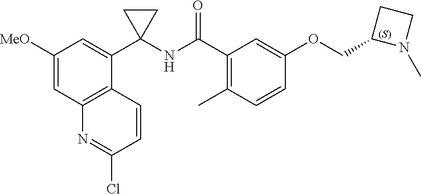 |
| 498 | 484 | 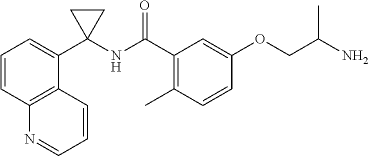 [SMILES: Cc1ccc(OCC(C)N)cc1C(=O)NC1(c2cccc3ncccc23)CC1] |
| 446 | 485 | 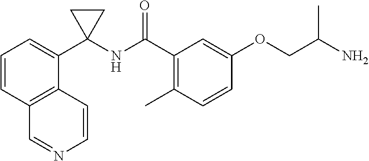 [SMILES: Cc1ccc(OCC(C)N)cc1C(=O)NC1(c2cccc3cnccc23)CC1] |
| 566 | 486 | 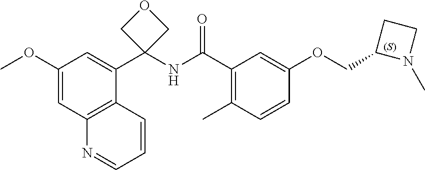 |
| 464 | 487 | 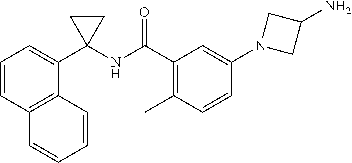 [SMILES: Cc1ccc(N2CC(N)C2)cc1C(=O)NC1(c2cccc3ccccc23)CC1] |
| 485 | 488 | 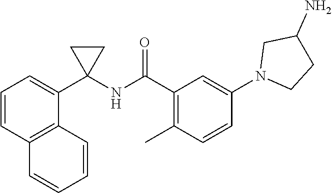 [SMILES: Cc1ccc(N2CCC(N)C2)cc1C(=O)NC1(c2cccc3ccccc23)CC1] |
| 506 | 489 | 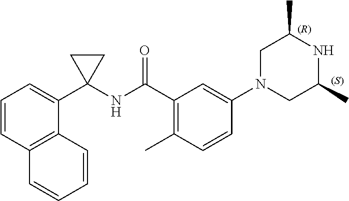 |
| 479 | 490 | 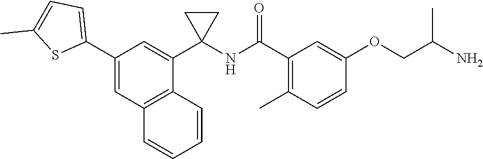 [SMILES: Cc1ccc(-c2cc(C3(NC(=O)c4cc(OCC(C)N)ccc4C)CC3)c3ccccc3c2)s1] |
| 502 | 491 | 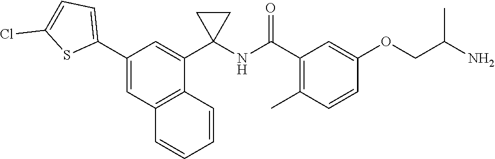 [SMILES: Cc1ccc(OCC(C)N)cc1C(=O)NC1(c2cc(-c3ccc(Cl)s3)cc3ccccc23)CC1] |
| 480 | 492 | 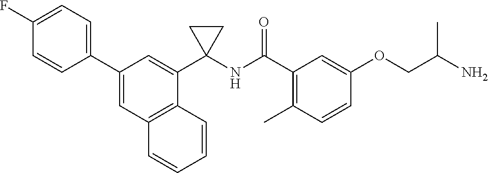 [SMILES: Cc1ccc(OCC(C)N)cc1C(=O)NC1(c2cc(-c3ccc(F)cc3)cc3ccccc23)CC1] |
| 478 | 493 | 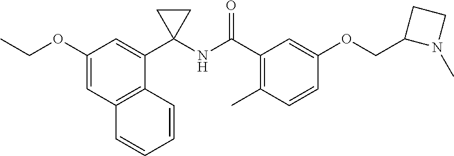 [SMILES: CCOc1cc(C2(NC(=O)c3cc(OCC4CCN4C)ccc3C)CC2)c2ccccc2c1] |
| 476 | 494 | 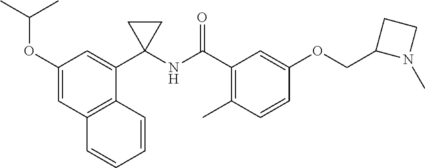 [SMILES: Cc1ccc(OCC2CCN2C)cc1C(=O)NC1(c2cc(OC(C)C)cc3ccccc23)CC1] |
| 787 | 495 | 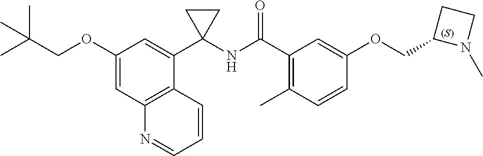 |
| 493 | 496 | 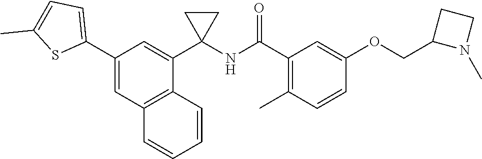 |
| 514 | 497 | 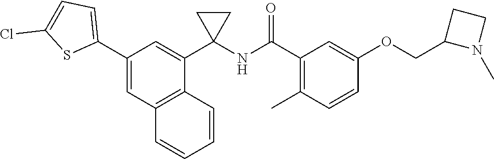 [SMILES: Cc1ccc(OCC2CCN2C)cc1C(=O)NC1(c2cc(-c3ccc(Cl)s3)cc3ccccc23)CC1] |
| 518 | 498 | 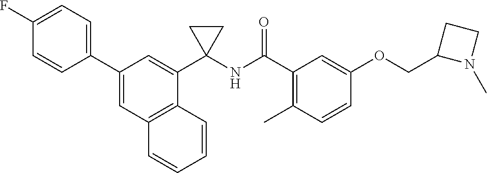 [SMILES: Cc1ccc(OCC2CCN2C)cc1C(=O)NC1(c2cc(-c3ccc(F)cc3)cc3ccccc23)CC1] |
| 500 | 499 | 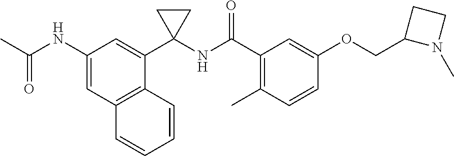 [SMILES: CC(=O)Nc1cc(C2(NC(=O)c3cc(OCC4CCN4C)ccc3C)CC2)c2ccccc2c1] |
| 510 | 500 | 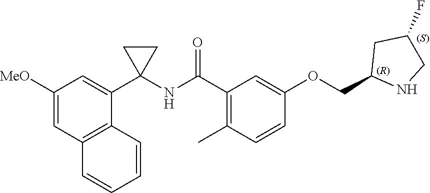 |
| 512 | 501 | 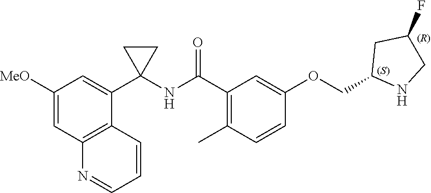 |
| 556 | 502 | 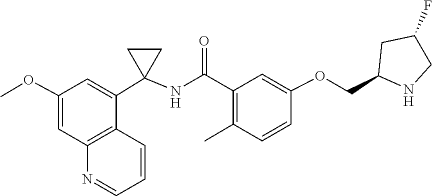 [SMILES: COc1cc(C2(NC(=O)c3cc(OC[C@H]4C[C@H](F)CN4)ccc3C)CC2)c2cccnc2c1] |
| 517 | 503 | 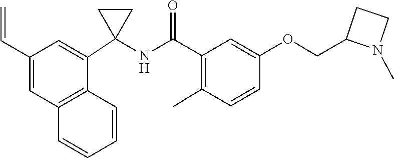 [SMILES: C=Cc1cc(C2(NC(=O)c3cc(OCC4CCN4C)ccc3C)CC2)c2ccccc2c1] |
| 503 | 504 | 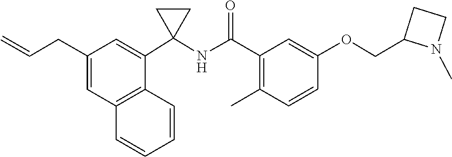 [SMILES: C=CCc1cc(C2(NC(=O)c3cc(OCC4CCN4C)ccc3C)CC2)c2ccccc2c1] |
| 578 | 505 | 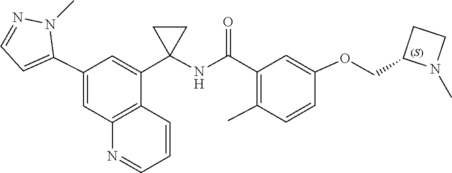 |
| 554 | 506 | 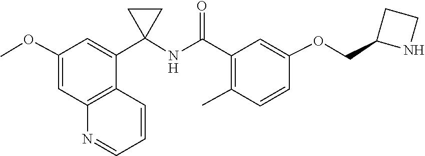 [SMILES: COc1cc(C2(NC(=O)c3cc(OC[C@H]4CCN4)ccc3C)CC2)c2cccnc2c1] |
| 516 | 507 | 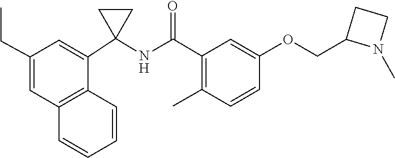 [SMILES: CCc1cc(C2(NC(=O)c3cc(OCC4CCN4C)ccc3C)CC2)c2ccccc2c1] |
| 504 | 508 | 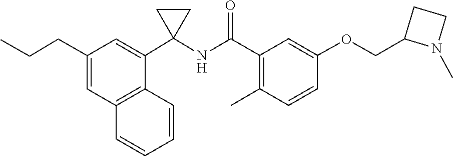 [SMILES: CCCc1cc(C2(NC(=O)c3cc(OCC4CCN4C)ccc3C)CC2)c2ccccc2c1] |
| 558 | 509 | 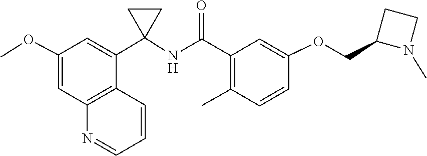 |
| 531 | 510 | 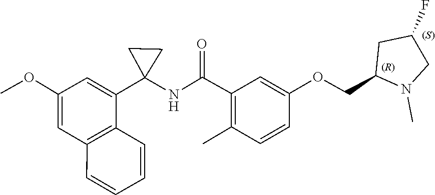 |
| 532 | 511 | 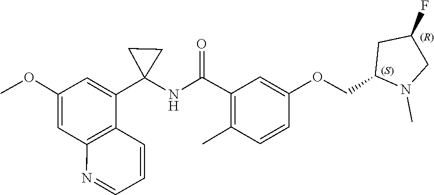 |
| 557 | 512 | 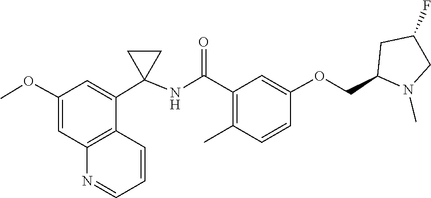 [SMILES: COc1cc(C2(NC(=O)c3cc(OC[C@H]4C[C@H](F)CN4C)ccc3C)CC2)c2cccnc2c1] |
| 553 | 513 | 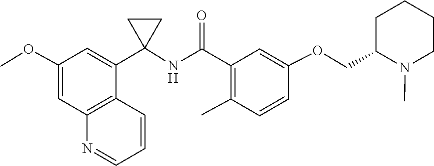 [SMILES: COc1cc(C2(NC(=O)c3cc(OC[C@@H]4CCCCN4C)ccc3C)CC2)c2cccnc2c1] |
| 535 | 514 | 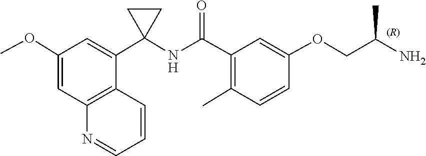 |
| 536 | 515 | 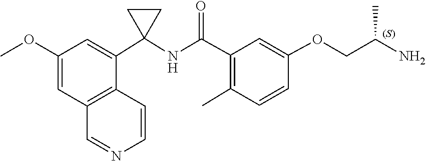 |
| 537 | 516 | 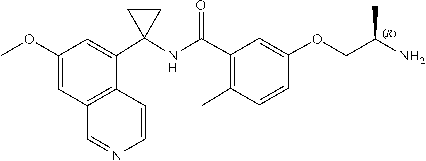 |
| 543 | 517 | 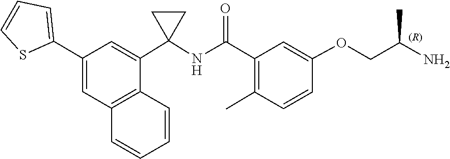 |
| 540 | 518 | 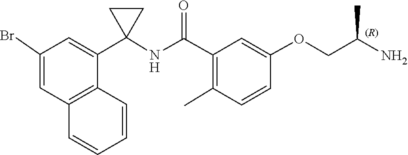 |
| 552 | 519 | 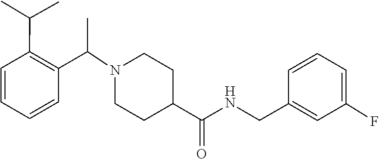 [SMILES: CC(C)c1ccccc1C(C)N1CCC(C(=O)NCc2cccc(F)c2)CC1] |
| 593 | 520 | 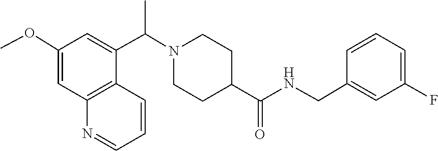 [SMILES: COc1cc(C(C)N2CCC(C(=O)NCc3cccc(F)c3)CC2)c2cccnc2c1] |
| 599 | 521 | 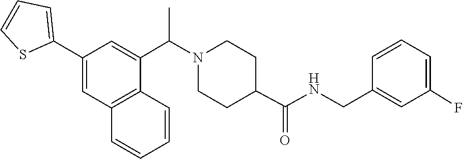 [SMILES: CC(c1cc(-c2cccs2)cc2ccccc12)N1CCC(C(=O)NCc2cccc(F)c2)CC1] |
| 573 | 522 | 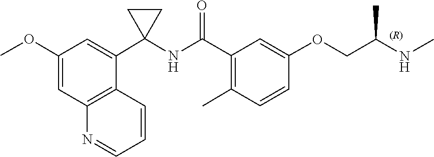 |
| 562 | 523 | 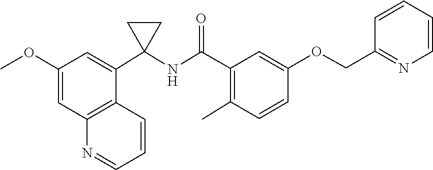 [SMILES: COc1cc(C2(NC(=O)c3cc(OCc4ccccn4)ccc3C)CC2)c2cccnc2c1] |
| 579 | 524 | 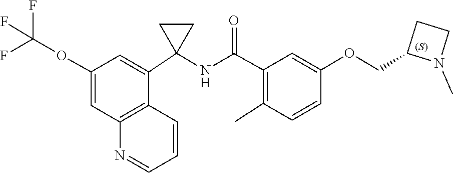 |
| 570 | 525 | 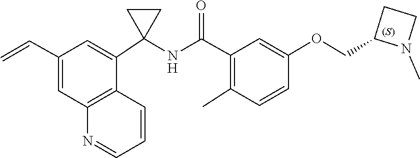 |
| 603 | 526 | 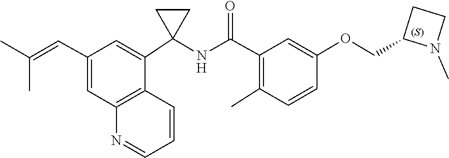 |
| 650 | 527 | 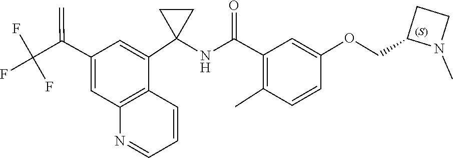 |
| 668 | 528 | 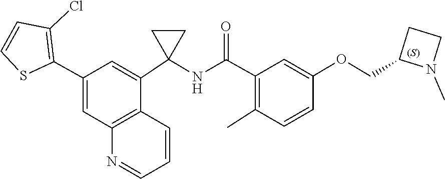 |
| 632 | 529 | 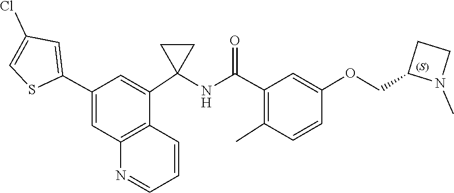 |
| 623 | 530 | 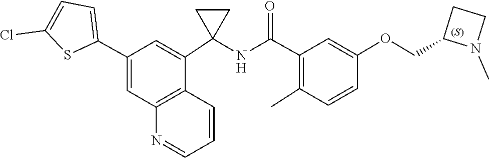 |
| 604 | 531 | 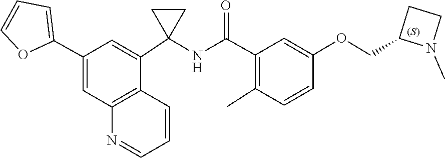 |
| 652 | 532 | 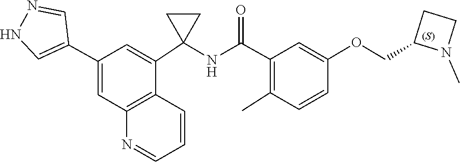 |
| 670 | 533 | 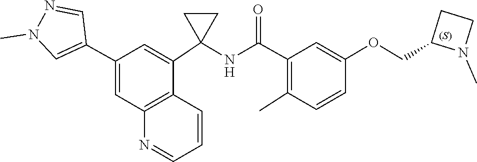 |
| 633 | 534 | 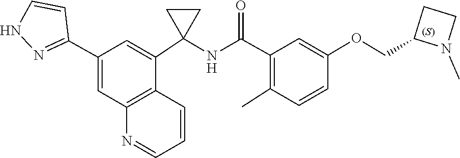 |
| 651 | 535 | 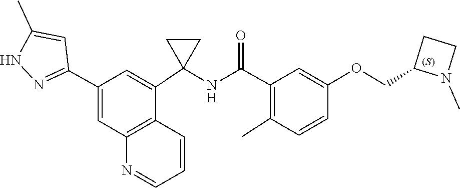 |
| 582 | 536 | 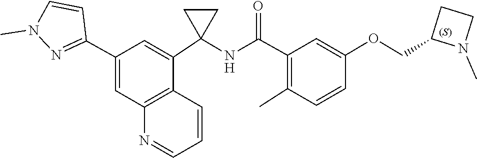 |
| 644 | 537 | 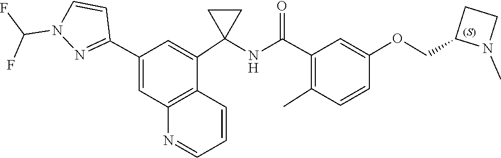 |
| 659 | 538 | 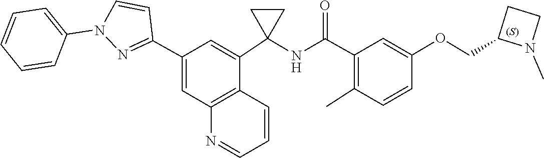 |
| 609 | 539 | 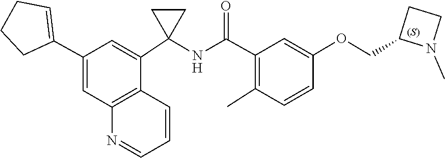 |
| 621 | 540 | 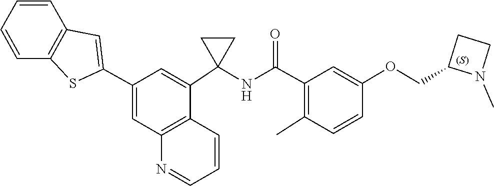 |
| 594 | 541 | 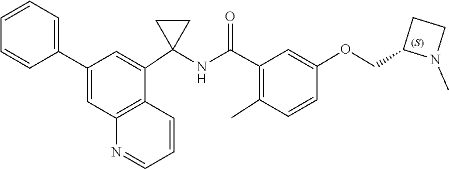 |
| 574 | 542 | 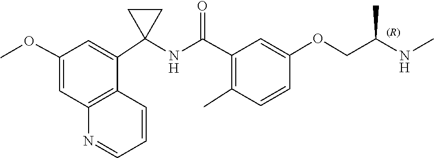 |
| 592 | 543 | 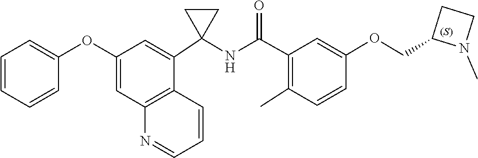 |
| 590 | 544 | 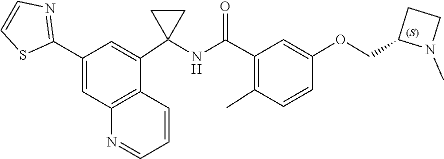 |
| 591 | 545 | 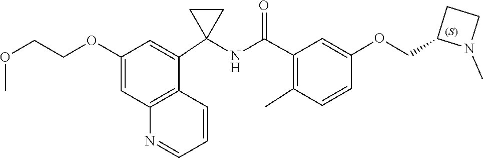 |
| 612 | 546 | 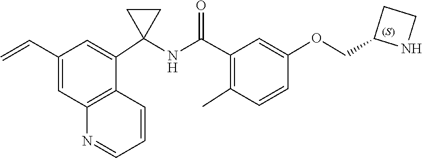 |
| 613 | 547 | 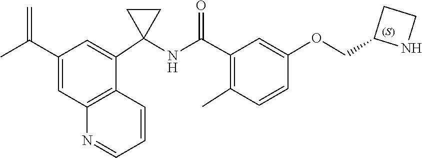 |
| 667 | 548 | 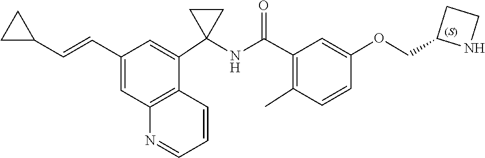 |
| 704 | 549 | 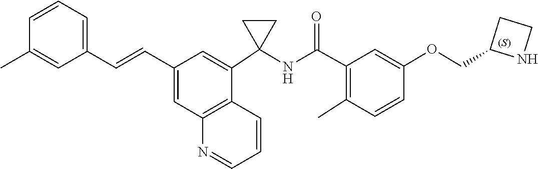 |
| 640 | 550 | 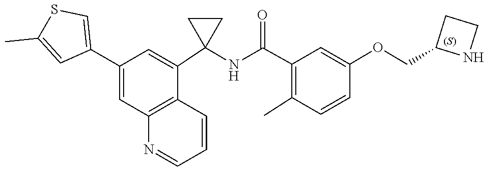 |
| 643 | 551 | 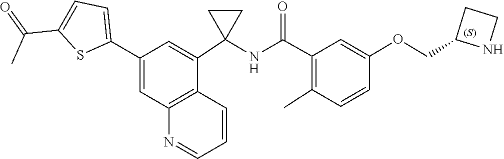 |
| 658 | 552 | 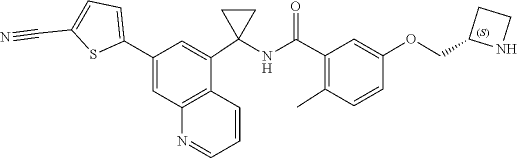 |
| 673 | 553 | 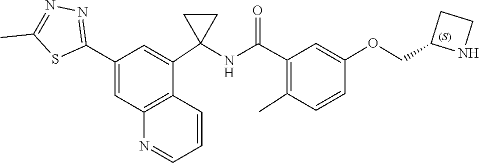 |
| 645 | 554 | 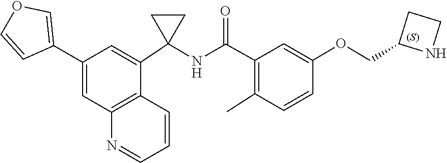 |
| 641 | 555 | 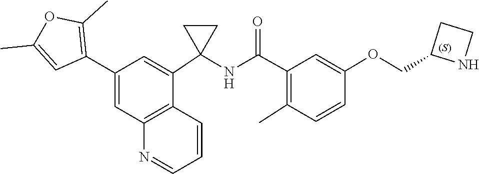 |
| 646 | 556 | 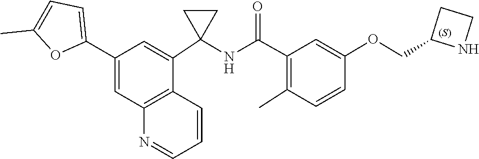 |
| 694 | 557 | 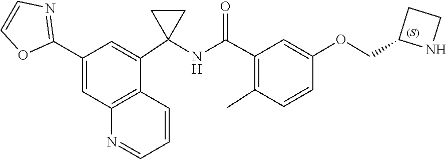 |
| 698 | 558 | 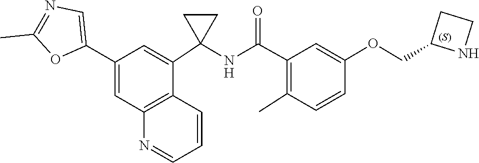 |
| 680 | 559 | 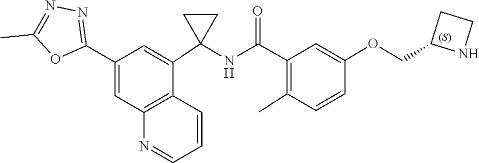 |
| 636 | 560 | 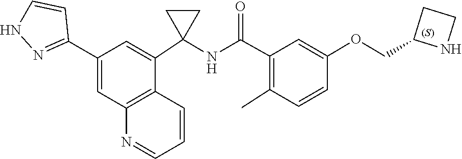 |
| 635 | 561 | 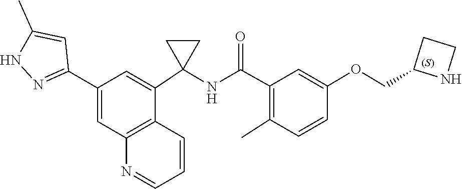 |
| 649 | 562 | 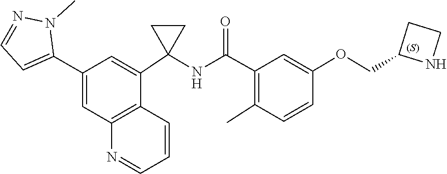 |
| 634 | 563 | 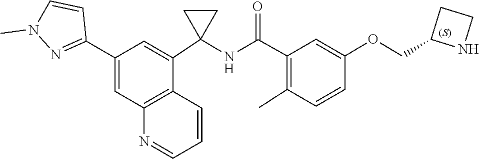 |
| 656 | 564 | 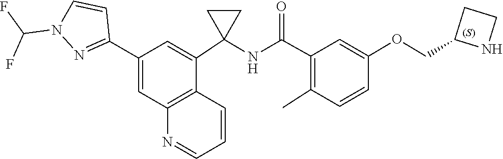 |
| 664 | 565 | 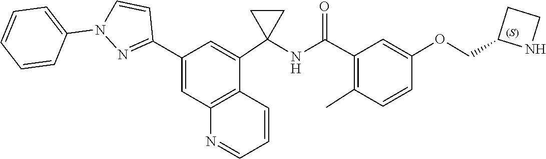 |
| 642 | 566 | 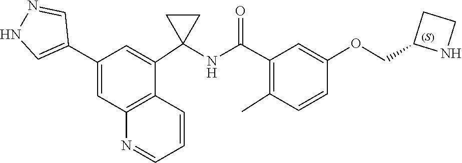 |
| 665 | 567 | 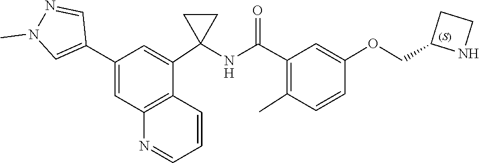 |
| 695 | 568 | 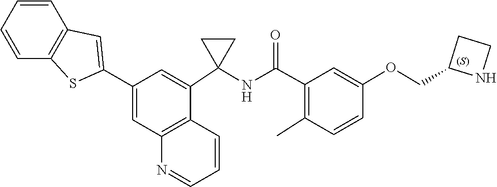 |
| 711 | 569 | 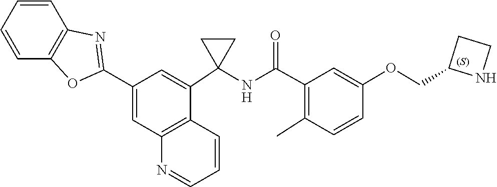 |
| 662 | 570 | 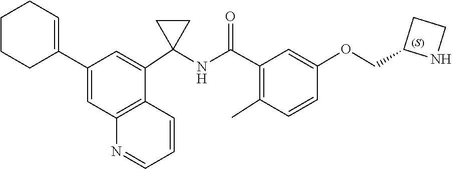 |
| 675 | 571 | 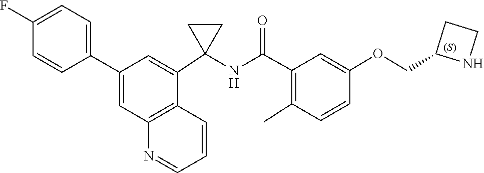 |
| 666 | 572 | 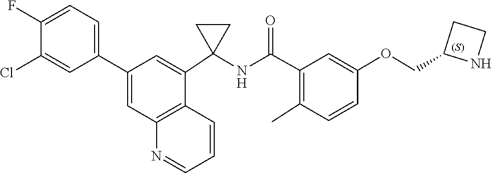 |
| 674 | 573 | 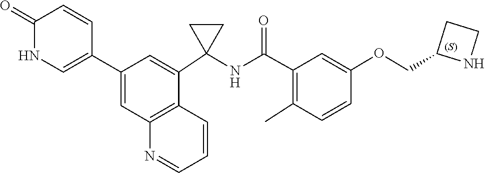 |
| 637 | 574 | 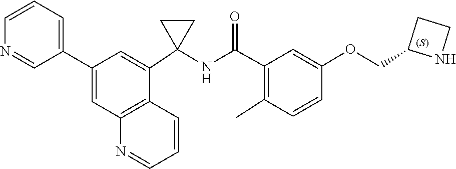 |
| 682 | 575 | 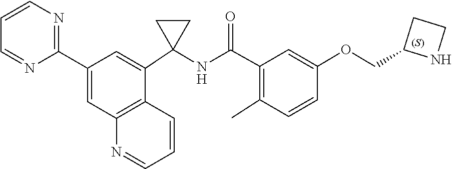 |
| 688 | 576 | 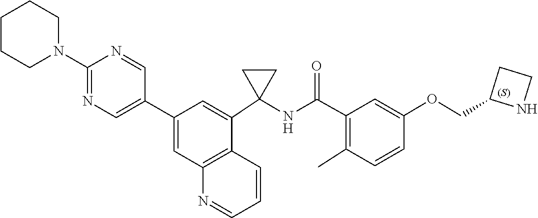 |
| 686 | 577 | 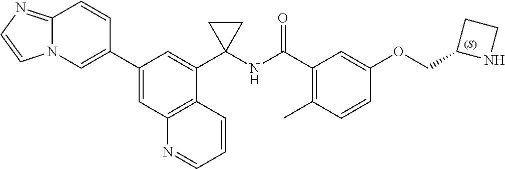 |
| 685 | 578 | 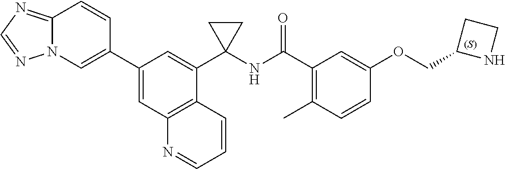 |
| 822 | 579 | 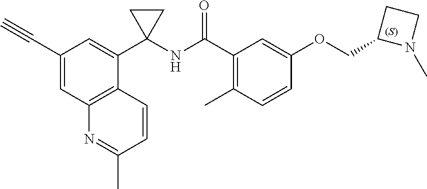 |
| 615 | 580 | 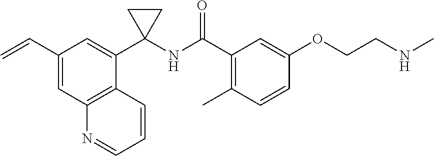 [SMILES: C=Cc1cc(C2(NC(=O)c3cc(OCCNC)ccc3C)CC2)c2cccnc2c1] |
| 616 | 581 | 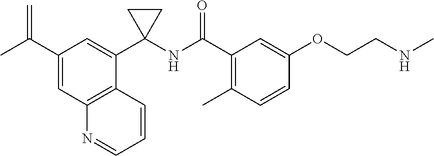 [SMILES: C=C(C)c1cc(C2(NC(=O)c3cc(OCCNC)ccc3C)CC2)c2cccnc2c1] |
| 639 | 582 | 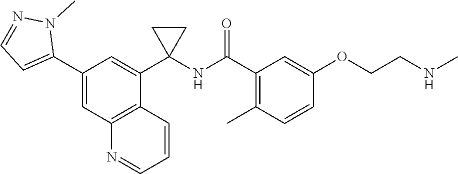 [SMILES: CNCCOc1ccc(C)c(C(=O)NC2(c3cc(-c4ccnn4C)cc4ncccc34)CC2)c1] |
| 647 | 583 | 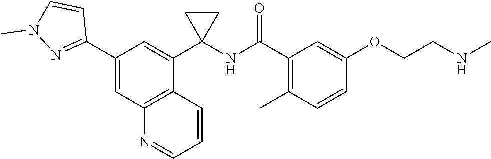 |
| 802 | 584 | 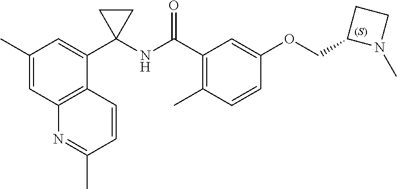 |
| 717 | 585 | 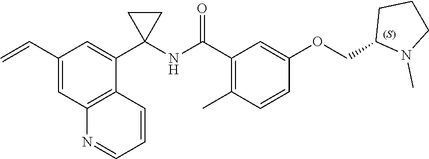 |
| 813 | 586 | 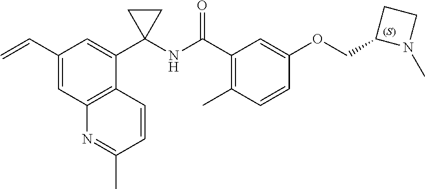 |
| 719 | 587 | 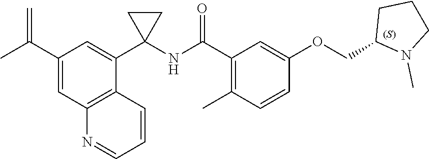 |
| 684 | 588 | 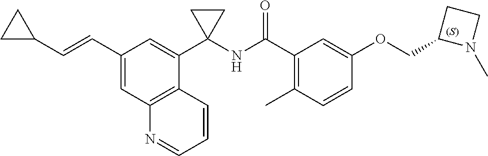 |
| 690 | 589 | 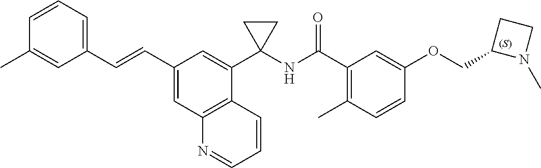 |
| 721 | 590 | 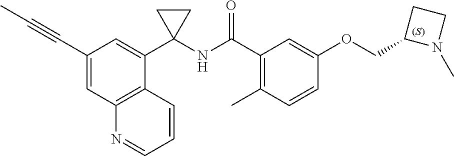 |
| 657 | 591 | 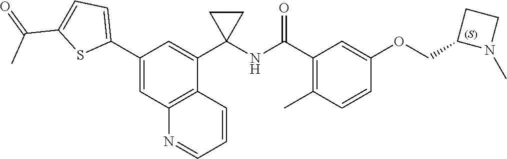 |
| 754 | 592 | 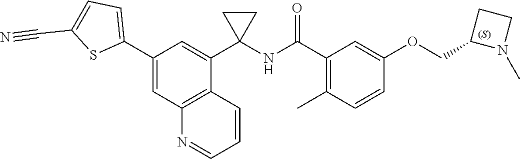 |
| 764 | 593 | 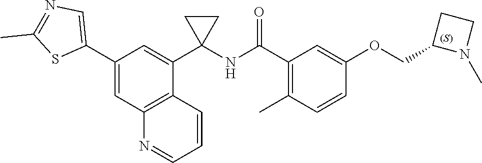 |
| 691 | 594 | 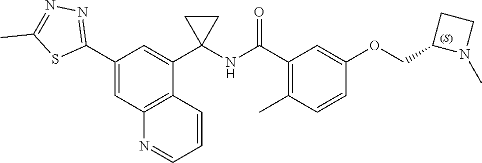 |
| 654 | 595 | 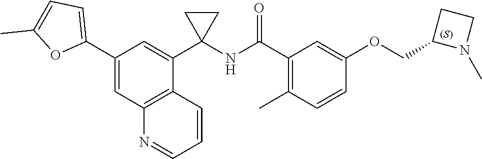 |
| 655 | 596 | 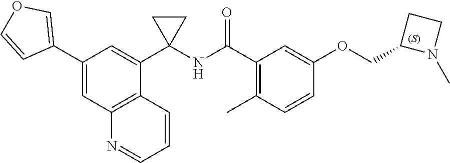 |
| 671 | 597 | 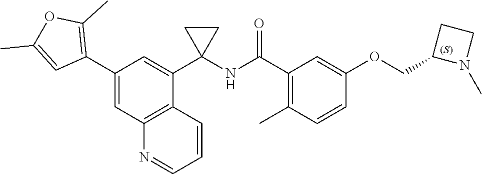 |
| 716 | 598 | 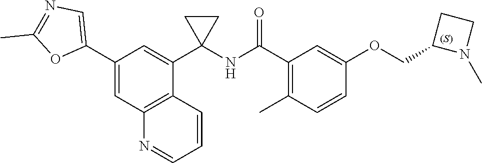 |
| 693 | 599 | 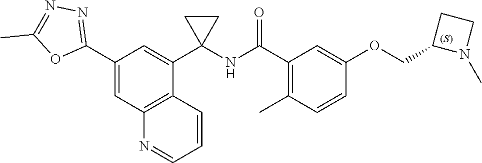 |
| 820 | 600 | 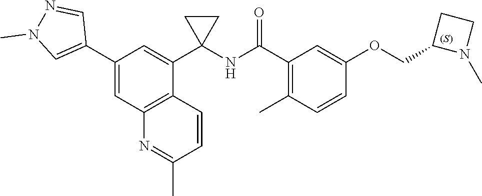 |
| 718 | 601 | 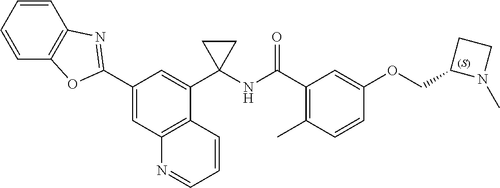 |
| 736 | 602 | 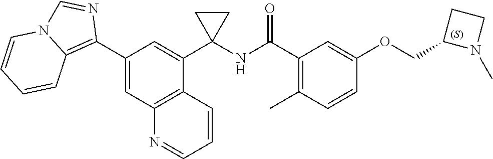 |
| 663 | 603 | 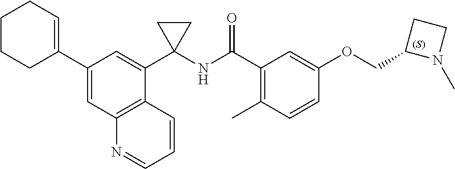 |
| 678 | 604 | 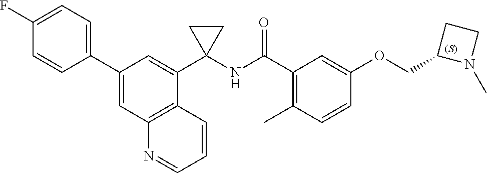 |
| 687 | 605 | 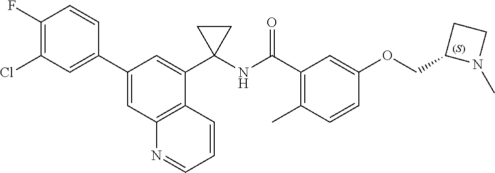 |
| 672 | 606 | 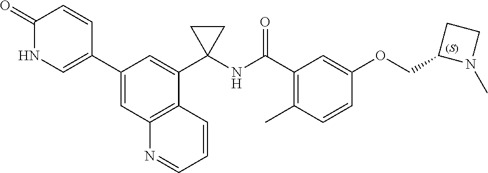 |
| 681 | 607 | 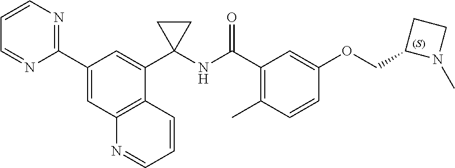 |
| 701 | 608 | 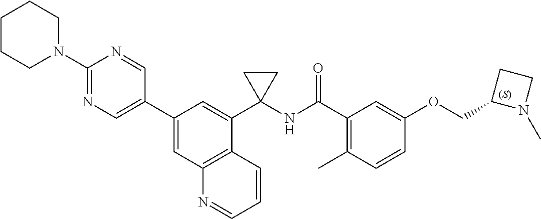 |
| 699 | 609 | 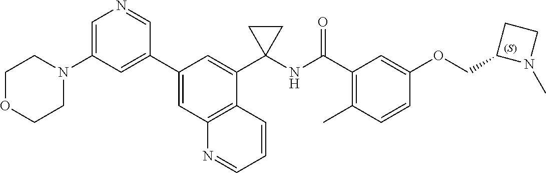 |
| 702 | 610 | 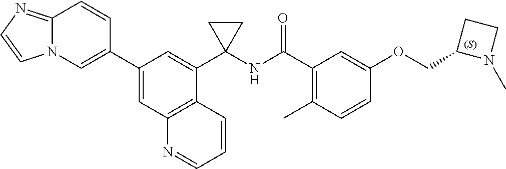 |
| 700 | 611 | 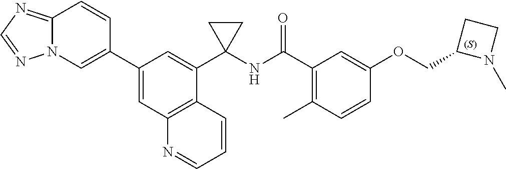 |
| 661 | 612 | 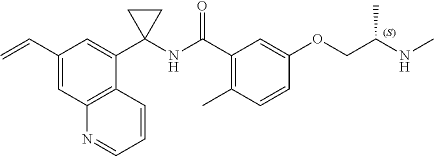 |
| 735 | 613 | 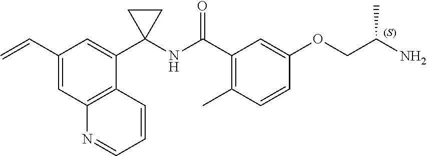 |
| 734 | 614 | 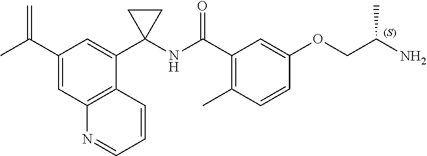 |
| 751 | 615 | 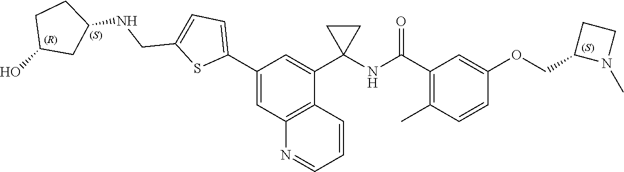 |
| 756 | 616 | 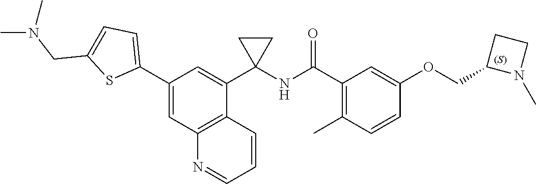 |
| 805 | 617 | 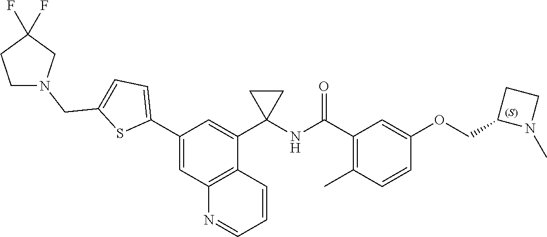 |
| 752 | 618 | 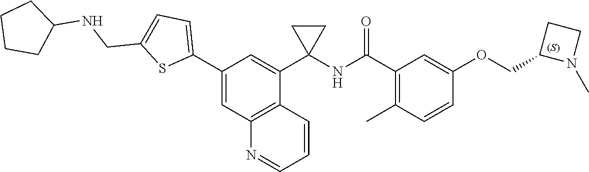 |
| 793 | 619 | 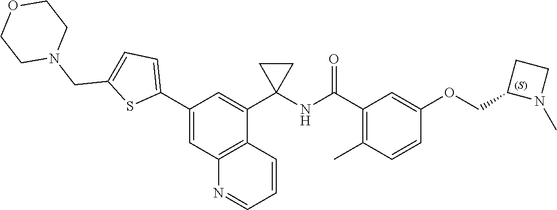 |
| 781 | 620 | 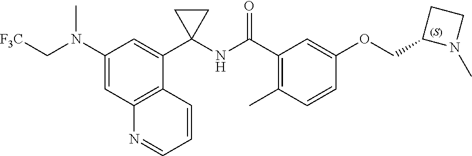 |
| 807 | 621 | 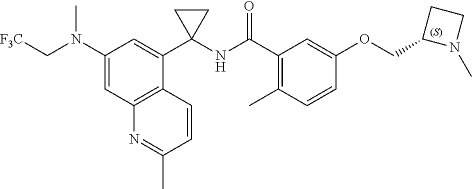 |
| 771 | 622 | 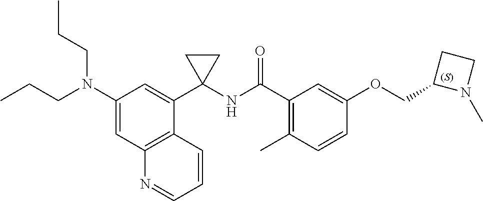 |
| 722 | 623 | 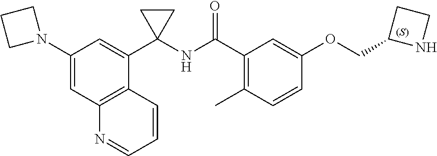 |
| 723 | 624 | 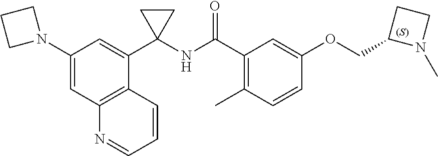 |
| 811 | 625 | 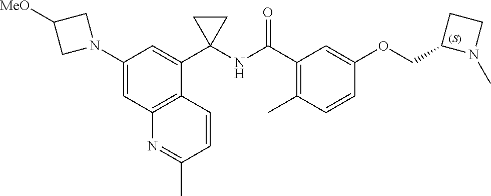 |
| 810 | 626 | 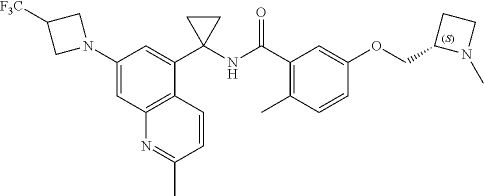 |
| 816 | 627 | 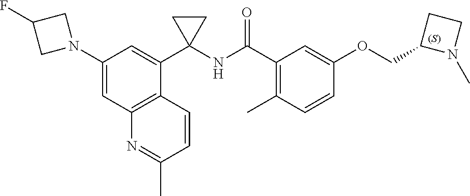 |
| 826 | 628 | 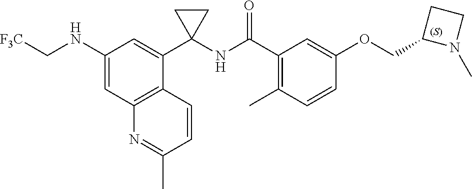 |
| 712 | 629 | 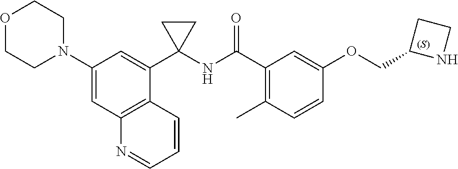 |
| 740 | 630 | 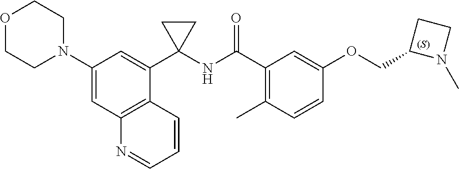 |
| 714 | 631 | 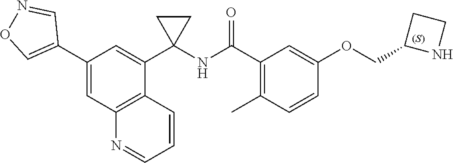 |
| 733 | 632 | 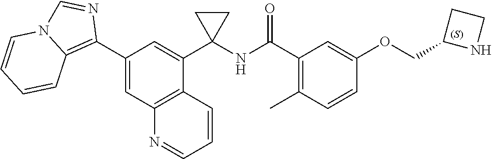 |
| 715 | 633 | 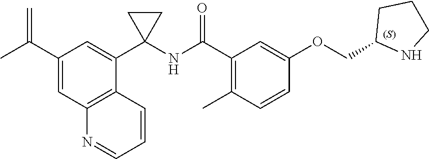 |
| 824 | 634 | 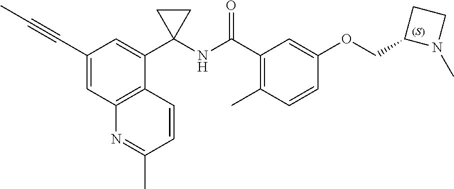 |
| 763 | 635 | 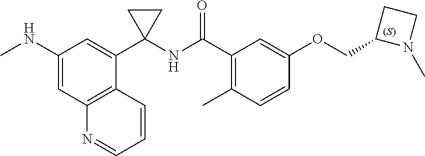 |
| 745 | 636 | 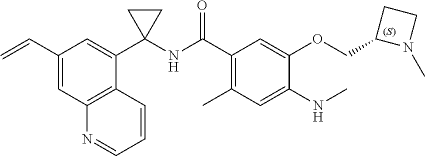 |
| 759 | 637 | 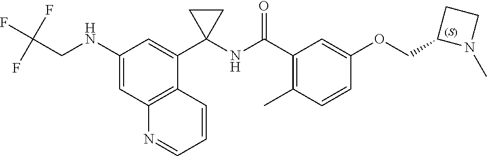 |
| 747 | 638 | 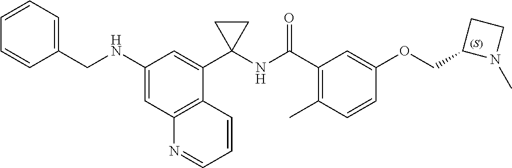 |
| 760 | 639 | 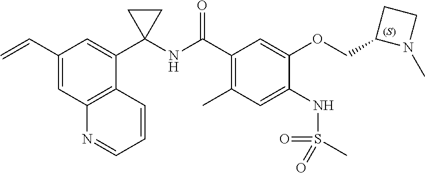 |
| 755 | 640 | 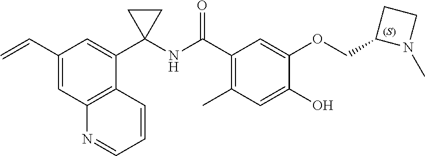 |
| 762 | 641 | 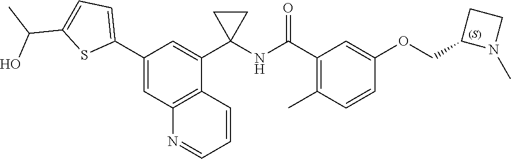 |
| 758 | 642 | 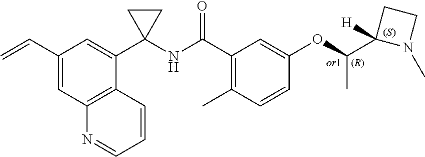 |
| 774 | 643 | 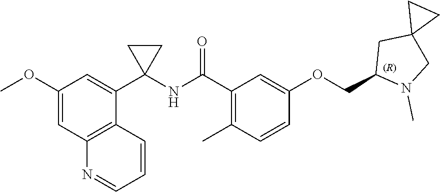 |
| 788 | 644 | 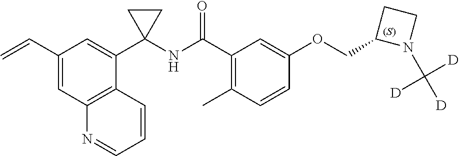 |
| 798 | 645 | 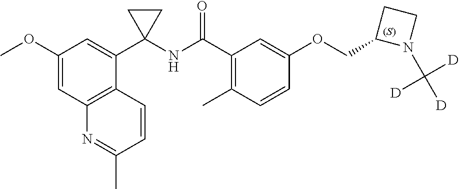 |
| 818 | 646 | 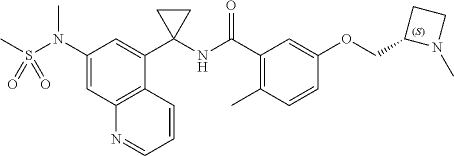 |
| 825 | 647 | 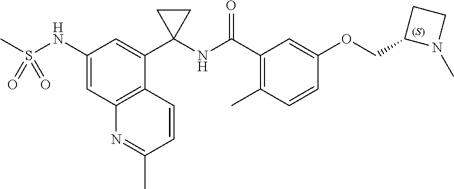 |
| 819 | 648 | 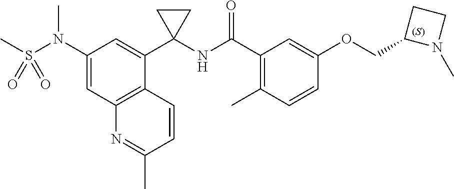 |
| 804 | 649 | 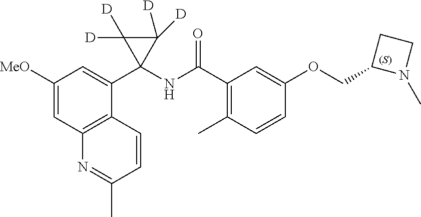 |
| 823 | 650 | 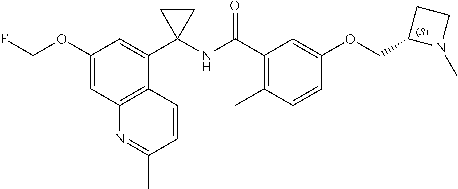 |
| 801 | 651 | 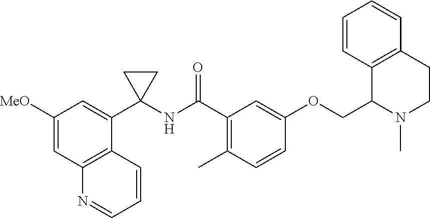 [SMILES: COc1cc(C2(NC(=O)c3cc(OCC4c5ccccc5CCN4C)ccc3C)CC2)c2cccnc2c1] |

### Pharmaceutical Compositions

Also disclosed herein are pharmaceutical compositions comprising a compound described herein or a pharmaceutically acceptable salt thereof, and one or more (e.g., one, two, three, or four) pharmaceutically acceptable excipients. In certain embodiments, the compound of the present invention is provided in an effective amount in the pharmaceutical composition. In certain embodiments, the compound of the present invention is provided in a therapeutically effective amount.

In certain embodiments, the pharmaceutical composition comprises an effective amount of the active ingredient. In certain embodiments, the pharmaceutical composition comprises a therapeutically effective amount of the active ingredient.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic and may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4 chlorobenzenesulfonic acid, 2-naphthalenesulfonic acid, 4-toluenesulfonic acid, camphorsulfonic acid, 4 methylbicyclo[2.2.2]oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. (See, e.g., Berge, et al., J. Pharm. Sci. (1977) 66 (1): 1-79, the entirety of the contents of which are incorporated by reference herein).

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, the “effective amount” of a compound refers to an amount sufficient to elicit the desired biological response, e.g., to treat a CNS-related disorder, is sufficient to induce anesthesia or sedation. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more (e.g., one, two, three, or four) symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Pharmaceutically acceptable excipients include any and all diluents or other liquid vehicles, dispersion or suspension aids, surface active agents, isotonic agents, preservatives, lubricants and the like, as suited to the particular dosage form desired. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

The compounds and pharmaceutical compositions provided herein can be administered as the sole active agent, or they can be administered in combination with other active agents.

In one aspect, the present invention provides a combination of a compound of the present invention and another pharmacologically active agent. Administration in combination can proceed by any technique apparent to those of skill in the art including, for example, separate, sequential, concurrent, and alternating administration.

### Methods of Treatment

Another aspect of the disclosure provides methods of treating patients suffering from a viral infection, e.g., a coronaviral infection. In particular, in certain embodiments, the disclosure provides a method of treating the below medical indications comprising administering to a subject in need thereof a therapeutically effective amount of a compound described herein, such as a compound described herein. In one aspect, the compounds described herein are contemplated as PLpro inhibitors. In certain embodiments, the disclosure provides a method of treating a viral infection in a patient in need thereof, comprising inhibiting PLpro by administering a compound described herein.

SARS-COV-2 is a positive strand RNA betacoronavirus. The genome of SARS-COV-2 contains 16 nonstructural proteins (Nsp1 to Nsp16) that are initially expressed as a viral polyprotein (See Thiel, et al., Journal of General Virology 2003, 84 (9), 2305-2315; Barretto, et al, J Virol 2005, 79 (24), 15189-98; and Harcourt, et al., Journal of Virology 2004, 78 (24), 13600-13612, the entirety of the contents of each of which are incorporated by reference herein). During viral replication in coronaviruses, this polyprotein is processed by viral-encoded proteases to facilitate the formation of the membrane-bound replicase complex that carries out viral RNA replication (See Mielech, et al, Journal of Virology 2015, 89 (9), 4907-4917, the entirety of the contents of which are incorporated by reference herein). One of the main proteases from CoV, the 3C-like protease, is known for its ability to cleave Nsp4-Nsp16. In addition to the 3C-like protease, CoVs can also encode up to two papain-like proteases (PLpros), one of which cleaves Nsp1-3. For example, CoVs such as the mouse hepatitis virus (MHV) and other human coronaviruses, including NL63, OC43, HKU1, and 229E, encode for PLP1 and PLP29. For SARS-COV-2, its genome mirrors that of the Middle East respiratory syndrome CoV (MERS-COV) and SARS-COV by coding for a single papain-like protease. Without being limited to a mechanism of action, the dual viral polypeptide cleavage and immune suppression roles of PLpros are potential targets for small molecule antiviral development. PLpro is discussed in Mclain and Vabret, Signal Transduction and Targeted Therapy 2020, 5:223; and Rut et al., Sci. Adv. 2020, 6: eabd4596, the entirety of the contents of each of which are incorporated by reference herein.

In some embodiments, the infection is chronic. As used herein, “chronic” refers to an infection that persists for an extended period of time, or recurs. In some embodiments, the infection is acute. As used herein, “acute” refers to an infection that is of short duration.

Methods to quantify viral replication are known in the art. In some embodiments, viral count is determined using a plaque assay. In some embodiments, viral count is determined using a focus forming assay (FFA). In some embodiments, viral count is determined using an endpoint dilution assay. In some embodiments, viral count is determined using an enzyme-linked-63-immunosorbent assay (ELISA). In some embodiments, viral count is determined using Tunable resistive pulse sensing (TRPS) to detect individual virus particles. In some embodiments, viral replication is determined by quantifying the amount or percentage of host cell death, e.g., in vitro, for example, using propidium iodide (PI) to identify dead cells, quantifying the amount of morphologically rounded cells, or by immunofluorescence microscopy for apoptotic markers. In some embodiments, viral count is determined by measuring viral titer or multiplicity of infection (MOI) or by performing a plaque assay, a focus forming assay, and endpoint dilution assay, a viral protein quantification assay (for example, a hemagglutination assay, a bicinchoninic acid assay (BCA), or a single radial immunodiffusion assay (SRID) assay), transmission electron microscopy analysis, a tunable resistive pulse sensing (TRPS) assay, a flow cytometry assay, a quantitative PCR (qPCR) assay, or an Enzyme-linked immunosorbent assay (ELISA). In some embodiments, viral replication is determined by quantification of viral nucleic acid (for example, viral DNA or viral RNA) content.

Methods to quantify viral transmission are known in the art. In some embodiments, viral transmission is quantified using epidemiological modeling (see, e.g., Graw F. et al., (2016) Modeling Viral Spread. Annu Rev Virol, 3 (1)). In some embodiments, viral transmission is assessed in vitro, e.g., in cell culture, e.g., using microscopy, e.g., using transmission electron microscopy (TEM).

Methods to quantify viral assembly are known in the art. In some embodiments, viral assembly is determined using statistical modeling (see, e.g., Clement N et al., (2018) Viral Capsid Assembly: A Quantified Uncertainty Approach. J Comp Biol, 25 (1)). In some embodiments, viral assembly is determined using biochemical techniques to determine capsid complex formation, e.g., co-immunoprecipitation, e.g., western blotting. In some embodiments, viral assembly is determined by flow cytometry for detection of colocalized viral protein (see, e.g., Stoffel, C. L. et al. (2005). “Rapid Determination of Baculovirus Titer by a Dual Channel Virus Counter” American Biotechnology Laboratory. 37 (22): 24-25).

Viral genes encode elements necessary for the process of viral infection, a multi-step process, including, for example, attachment to the host cell, penetration, de-envelopment, viral gene transcription cascade, viral protein expression, viral genome replication, viral packaging and assembly, envelopment, transport and maturation, release and egress, and host cell-to-cell transmission. β genes are those genes corresponding to early steps of viral infection, e.g., viral genome replication. γ genes are those genes corresponding to late steps of viral infection, e.g., egress. Methods to quantify viral gene expression are known in the art. In some embodiments, viral gene expression is determined using reverse transcriptase and quantitative polymerase chain reaction (RT-qPCR). In some embodiments, RNA sequencing (RNA-Seq) is used to determine viral gene expression. In some embodiments, viral DNA is quantified using a Southern blot. In some embodiments, β gene expression is quantified. In some embodiments, γ gene expression is quantified. In some embodiments, β gene expression and γ gene expression are quantified. In some embodiments, expression of the entire viral genome is quantified.

Methods to quantify virus release are known in the art. In some embodiments, viral release is determined by biochemical assay, e.g., western blotting, e.g., metabolic labeling (see, e.g., Yadav et al., (2012). “A facile quantitative assay for viral particle genesis reveals cooperativity in virion assembly and saturation of an antiviral protein.” Virology. 429 (2): 155-162). In some embodiments, viral release is determined by ELISA. In some embodiments, viral release is determined using electron microscopy, e.g., transmission electron microscopy (TEM). In some embodiments, viral release is determined by infectivity measurements for the detection of virions in a sample, e.g., serum. In some embodiments, viral release is determined by quantification of viral DNA or viral RNA in serum in vivo or culture supernatant in vitro.

Methods of treatment of the present invention can be used as a monotherapy or in combination with one or more (e.g., one, two, three, or four) other therapies (for example, anti-infective agents) that can be used to treat a disease or disorder, for example, an infection. The term “combination,” as used herein, is understood to mean that two or more different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Accordingly, in certain embodiments, the subject has received, is receiving, or is scheduled to receive one or more (e.g., one, two, three, or four) other therapies suitable for use in treating the disease or disorder. In certain embodiments, the method of treatment of the present invention further comprises administering to the subject one or more (e.g., one, two, three, or four) other therapies suitable for use in treating a disease or disorder, for example, an infection. In certain embodiments, the one or more (e.g., one, two, three, or four) other therapies comprise an agent that ameliorates one or more (e.g., one, two, three, or four) symptoms of infection with an intracellular pathogen. In certain embodiments, the one or more (e.g., one, two, three, or four) other therapies comprise surgical removal of an infected tissue.

Accordingly, in certain embodiments, the subject has received, is receiving, or is scheduled to receive one or more (e.g., one, two, three, or four) other therapies suitable for use in treating the disease or disorder. In certain embodiments, the method of treatment of the present invention further comprises administering to the subject one or more (e.g., one, two, three, or four) other therapies suitable for use in treating a disease or disorder, for example, an infection. In certain embodiments, the one or more (e.g., one, two, three, or four) other therapies comprise an agent that ameliorates one or more (e.g., one, two, three, or four) symptoms of infection with an intracellular pathogen. In certain embodiments, the one or more (e.g., one, two, three, or four) other therapies comprise surgical removal of an infected tissue.

It is understood that a method of use disclosed herein can be used in combination with an agent, for example, an anti-infective agent that ameliorates one or more (e.g., one, two, three, or four) symptoms of a disease or disorder associated with an intracellular pathogen. For example, a method of use disclosed herein can be used in combination with another antiviral agent.

In some embodiments, the additional therapeutic agents can be therapeutic anti-viral vaccines.

### Synthetic Procedures

### List of Abbreviations

| Abbreviation list | Full name | CAS number |
|---|---|---|
| ACN | Acetonitrile | 75-05-8 |
| BnBr | Benzyl bromide | 100-39-0 |
| Boc₂O | di-tert-Butyl dicarbonate | 24424-99-5 |
| CDI | 1,1′-Carbonyldiimidazole | 530-62-1 |
| CMBP | 2-(Tributylphosphoranylidene)acetonitrile | 157141-27-0 |
| DABCO | 1,4-Diazabicyclo[2.2.2]octane | 280-57-9 |
| DCC | Dicyclohexylcarbodiimide | 538-75-0 |
| DCE | 1,2-Dichloroethane | 107-06-2 |
| DCM | Dichloromethane | 75-09-2 |
| DIAD | Diisopropyl azodicarboxylate | 2446-83-5 |
| DIEA | N-Ethyl-N,N-diisopropylamine | 7087-68-5 |
| DMA | N,N-Dimethylacetamide | 127-19-5 |
| DME | 1,2-Dimethoxyethane | 110-71-4 |
| DPPF | 1,1′-Bis(diphenylphosphino)ferrocene | 12150-46-8 |
| EDCI | N-(3-Dimethylaminopropyl)-N- | 1892-57-5 |
| | ethylcarbodiimide | |
| HATU | N-[(Dimethylamino)-3-oxo-1H-1,2,3- | 148893-10-1 |
| | triazolo[4,5-b]pyridin-1-yl-methylene]-N- | |
| | methylmethanaminium hexafluorophosphate | |
| HOBt | 1-Hydroxybenzotriazole | 2592-95-2 |
| m-CPBA | 3-Chloroperoxybenzoic acid | 937-14-4 |
| MsCl | Methanesulfonyl chloride | 124-63-0 |
| MTBE | Methyl tert-butyl ether | 1634-04-4 |
| NIS | N-Iodosuccinimide | 516-12-1 |
| NMI | 1-Methyl-1H-imidazole | 616-47-7 |
| NMM | 4-Methylmorpholine | 109-02-4 |
| PCy₃ | Tricyclohexylphosphine | 2622-14-2 |
| Pd(dppf)Cl₂ | (1,1′- | 72287-26-4 |
| | Bis(diphenylphosphino)ferrocene)palladium(II) | |
| | dichloride | |
| Pd(dtbupf)Cl₂ | Dichloro[1,1′-bis(di-tert- | 95408-45-0 |
| | butylphosphino)ferrocene]palladium(II) | |
| Py | Pyridine | |
| SEMCl | 2-(Trimethylsilyl)ethoxymethyl chloride | 76513-69-4 |
| T₃P | 2,4,6-Tripropyl-1,3,5,2,4,6-trioxatriphosphinane- | 68957-94-8 |
| | 2,4,6-trioxide | |
| TBAC | Tetrabutylammonium chloride | 1112-67-0 |
| TBAF | Tetrabutylammonium fluoride | 429-41-4 |
| tBuXPhos Pd G3 | Methanesulfonato(2-di-tert-butylphosphino- | 1447963-75-8 |
| | 2,4,6-triisopropyl-1,1-biphenyl)(2-amino-1,1- | |
| | biphenyl-2-yl)palladium(II) | |
| TCFH | [Chloro(dimethylamino)methylidene]- | 94790-35-9 |
| | dimethylazanium;hexafluorophosphate | |
| TEA | Triethylamine | 121-44-8 |
| Tf₂O | Trifluoromethanesulfonic anhydride | 358-23-6 |
| TFA | Trifluoroacetic acid | 76-05-1 |
| TFE | 2,2,2-Trifluoroethanol | 132248-58-9 |
| THE | Tetrahydrofuran | |
| TMAD | (3E)-3-(Dimethylcarbamoylimino)-1,1- | 10465-78-8 |
| | dimethylurea | |
| TMSN₃ | Trimethylsilyl azide | 4648-54-8 |
| TsOH•H₂O | para-Toluenesulfonic acid monohydrate | 6192-52-5 |
| Xantphos | 4,5-Bis(diphenylphosphino)-9,9- | 161265-03-8 |
| | dimethylxanthene | |

### Example 1: 5-(Azetidin-3-ylamino)-2-methyl-N-(1-(5,6,7,8-tetrahydronaphthalen-1-yl)ethyl)benzamide (Compound 172)

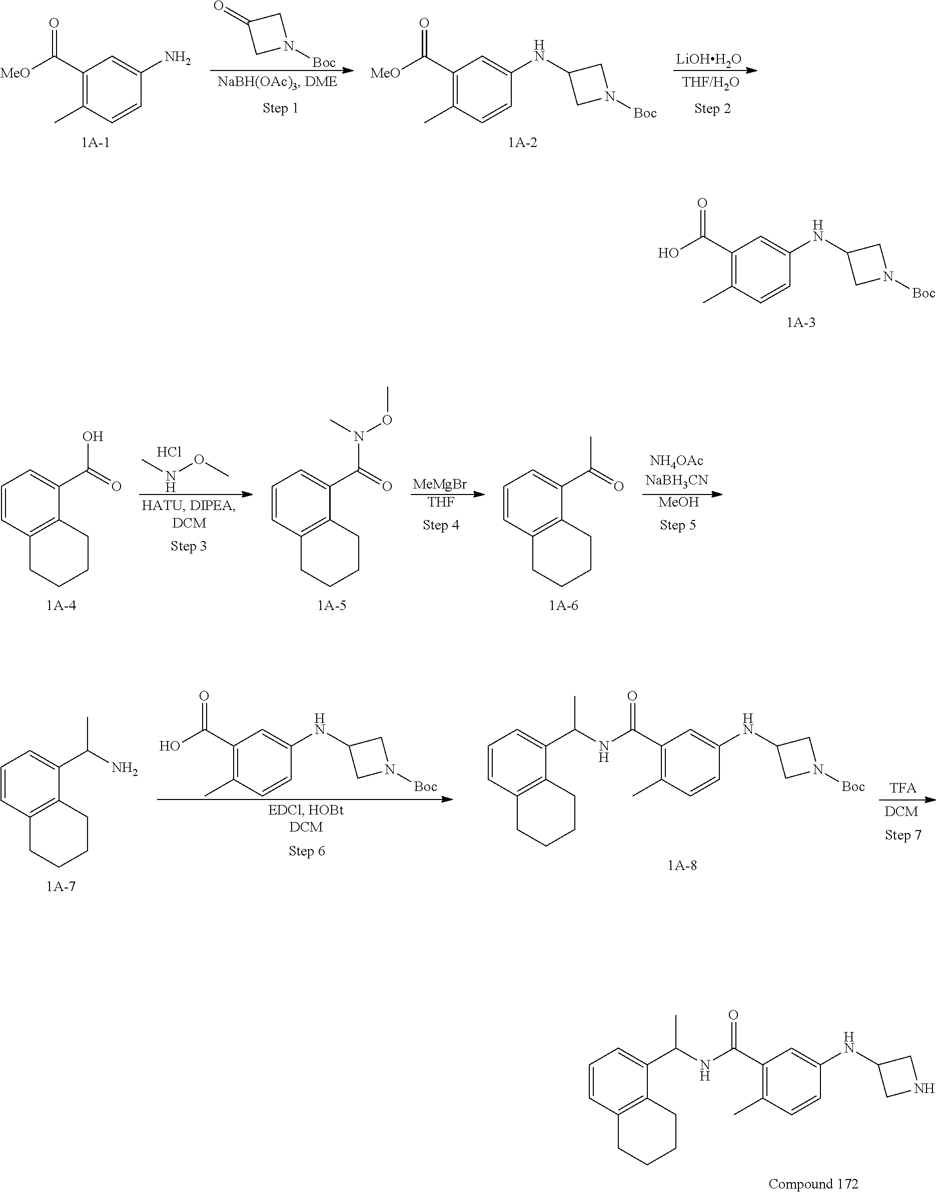

Step 1: tert-Butyl 3-((3-(methoxycarbonyl)-4-methylphenyl)amino)azetidine-1-carboxylate (1A-2)

To a solution of methyl 5-amino-2-methylbenzoate (1.10 g, 6.66 mmol, 1.1 eq) and tert-butyl 3-oxoazetidine-1-carboxylate (1.00 g, 5.84 mmol, 1.0 eq) in DME (30 mL) was added NaBH(OAc) 3 (1.49 g, 7.01 mmol, 1.2 eq). The mixture was stirred at 20° C. for 12 h. LCMS indicated that the starting material was completely consumed. The mixture was treated with water (40 mL) and extracted with DCM (20 mL×2). The combined organic layers were dried over Na₂SO₄, filtered, and concentrated under vacuum to give a residue which was purified by flash silica gel chromatography using a gradient of EtOAc/petroleum ether from 0/1 to 1/2. tert-Butyl 3-((3-(methoxycarbonyl)-4-methylphenyl)amino)azetidine-1-carboxylate (700 mg, 2.18 mmol, 37% yield) was obtained as a white solid. M−56+H⁺=265.2.

### Step 2: 5-((1-(tert-Butoxycarbonyl)azetidin-3-yl)amino)-2-methylbenzoic acid (1A-3)

To a solution of tert-butyl 3-((3-(methoxycarbonyl)-4-methylphenyl)amino)azetidine-1-carboxylate (700 mg, 2.18 mmol, 1.0 eq) in a mixture of H₂O (10 mL) and THF (30 mL) was added LiOH·H₂O (458 mg, 10.9 mmol, 5.0 eq). The mixture was stirred at 70° C. for 16 h. TLC indicated that the starting material was completely consumed, and a main spot was detected. The reaction mixture was allowed to cool to room temperature, poured into H₂O (10 mL) and washed with TMBE (10 mL×2). The aqueous layer was acidified to pH 5 with HCl (1 M aqueous). The product was extracted with 2-methyltetrahydrofuran (10 mL×3). The combined organic layers were dried over Na₂SO₄, filtered, and concentrated under vacuum to give crude product 5-((1-(tert-butoxycarbonyl)azetidin-3-yl)amino)-2-methylbenzoic acid (700 mg) as a white solid.

### Step 3: N-Methoxy-N-methyl-5,6,7,8-tetrahydronaphthalene-1-carboxamide (1A-5)

To a solution of N,O)-dimethylhydroxylamine hydrochloride (775 mg, 7.95 mmol, 2.0 eq) in DCM (15 mL) was added 5,6,7,8-tetrahydronaphthalene-1-carboxylic acid (700 mg, 3.97 mmol, 1.0 eq), followed by HATU (1.81 g, 4.76 mmol, 1.2 eq) and DIEA (1.95 g, 15.1 mmol, 2.63 mL, 3.8 eq). The resulting mixture was stirred at 20° C. for 16 h. LCMS indicated that the starting material was completely consumed. The mixture was treated with saturated aqueous NaHCO₃ (10 mL) and extracted with DCM (5.0 mL×2). The combined organic layers were dried over Na₂SO₄, filtered, and concentrated under vacuum to give a residue which was purified by flash silica gel chromatography using a gradient of EtOAc/petroleum ether from 0/1 to 1/2. N-Methoxy-N-methyl-5,6,7,8-tetrahydronaphthalene-1-carboxamide (440 mg, 2.01 mmol, 51% yield) was obtained as a colorless oil. M+H⁺=220.3 (LCMS); ¹H NMR (400 MHz, CDCl₃) δ 7.15-7.01 (m, 3H), 4.00-2.96 (m, 7H), 2.85-2.68 (m, 4H), 1.81-1.77 (m, 3H).

### Step 4: 1-(5,6,7,8-Tetrahydronaphthalen-1-yl)ethanone (1A-6)

To a solution of N-methoxy-N-methyl-5,6,7,8-tetrahydronaphthalene-1-carboxamide (400 mg, 1.82 mmol, 1.0 eq) in THF (10 mL) at 0° C. was added MeMgBr (3 M in Et₂O, 912 μL, 1.5 eq). The resulting mixture was stirred at 20° C. under a N₂ atmosphere for 16 h. LCMS indicated that the starting material was consumed, and the desired mass was detected. The mixture was treated with water (10 mL) and extracted with DCM (10 mL×5). The combined organic layers were dried over Na₂SO₄, filtered, and concentrated under vacuum to give a residue which was purified by flash silica gel chromatography using a gradient of EtOAc/petroleum ether from 0/1 to 1/5. 1-(5,6,7,8-Tetrahydronaphthalen-1-yl)ethanone (120 mg, 689 μmol, 38% yield) was obtained as colorless oil. ¹H NMR (400 MHZ, CDCl₃) δ 7.49-7.41 (m, 1H), 7.24-7.12 (m, 2H), 3.00-2.92 (m, 2H), 2.88-2.76 (m, 2H), 2.60-2.52 (m, 3H), 1.85-1.70 (m, 4H).

### Step 5: 1-(5,6,7,8-Tetrahydronaphthalen-1-yl)ethanamine (1A-7)

To a solution of 1-(5,6,7,8-tetrahydronaphthalen-1-yl)ethanone (100 mg, 574 μmol, 1.0 eq) in MeOH (6.0 mL) was added NH₄OAc (531 mg, 6.88 mmol, 12 eq), followed by NaBH₃CN (144 mg, 2.30 mmol, 4.0 eq). The resulting mixture was stirred at 60° C. for 16 h. LCMS indicated that the starting material was completely consumed. The mixture was treated with water (10 mL) and extracted with DCM (5.0 mL×5). The combined organic layers were dried over Na₂SO₄, filtered, and concentrated under vacuum to give 1-(5,6,7,8-tetrahydronaphthalen-1-yl)ethanamine (90.0 mg, 513 μmol, 89% yield) as a colorless oil. M−17+H⁺=159.0 (LCMS).

### Step 6: tert-Butyl 3-((4-methyl-3-((1-(5,6,7,8-tetrahydronaphthalen-1-yl)ethyl)carbamoyl)phenyl)amino)azetidine-1-carboxylate (1A-8)

To a solution of 1-(5,6,7,8-tetrahydronaphthalen-1-yl)ethanamine (60.0 mg, 342 μmol, 1.0 eq) and 5-((1-(tert-butoxycarbonyl)azetidin-3-yl)amino)-2-methylbenzoic acid (94.4 mg, 308 μmol, 0.9 eq) in DCM (5.0 mL) were added EDCI (98.4 mg, 514 μmol, 1.5 eq), HOBt (69.4 mg, 514 μmol, 1.5 eq) and TEA (69.3 mg, 685 μmol, 95.3 μL, 2.0 eq). The mixture was stirred at 20° C. for 16 h. LCMS indicated that the starting material was completely consumed. The mixture was treated with water (10 mL) and extracted with DCM (5.0 mL×5). The combined organic layers were dried over Na₂SO₄, filtered, and concentrated under vacuum to give a residue which was purified by flash silica gel chromatography using a gradient of EtOAc/petroleum ether from 0/1 to 1/2. tert-Butyl 3-((4-methyl-3-((1-(5,6,7,8-tetrahydro naphthalen-1-yl)ethyl)carbamoyl)phenyl)amino)azetidine-1-carboxylate (80.0 mg, 162 μmol, 47%) was obtained as a yellow solid. M+Na⁺=486.1 (LCMS); ¹H NMR (400 MHZ, CD₃OD) δ 7.23 (d, J=7.1 Hz, 1H), 7.12-7.07 (m, 1H), 7.03-6.95 (m, 2H), 6.56-6.49 (m, 2H), 5.43-5.36 (m, 1H), 4.61-4.58 (m, 1H), 4.29-4.17 (m, 3H), 3.74-3.69 (m, 2H), 3.10-3.02 (m, 1H), 2.83-2.77 (m, 3H), 2.21-2.18 (m, 3H), 1.95-1.87 (m, 2H), 1.83-1.77 (m, 2H), 1.48 (m, 11H).

### Step 7: 5-(Azetidin-3-ylamino)-2-methyl-N-(1-(5,6,7,8-tetrahydronaphthalen-1-yl)ethyl)benzamide (Compound 172)

To a solution of tert-butyl 3-((4-methyl-3-((1-(5,6,7,8-tetrahydronaphthalen-1-yl)ethyl) carbamoyl)phenyl)amino)azetidine-1-carboxylate (60.0 mg, 129 μmol, 1.0 eq) in DCM (8.0 mL) was added TFA (4.62 g, 40.5 mmol, 3.00 mL, 313 eq). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Gemini C18 column (150×30 mm, 5 μm); flow rate: 25 mL/min; gradient: 10%-45% B over 8 min; mobile phase A: 0.1% aqueous TFA, mobile phase B: acetonitrile) to give 5-(azetidin-3-ylamino)-2-methyl-N-(1-(5,6,7,8-tetrahydronaphthalen-1-yl)ethyl)benzamide (22.7 mg, 47.6 μmol, 37% yield, TFA salt) as a yellow solid. M+H⁺=364.3 (LCMS); ¹H NMR (400 MHZ, CD₃OD) δ 7.22-7.18 (m, 1H), 7.10-7.00 (m, 2H), 6.98-6.94 (m, 1H), 6.58-6.53 (m, 1H), 6.50-6.48 (m, 1H), 5.41-5.38 (m, 1H), 4.50-4.45 (m, 1H), 4.38-4.32 (m, 2H), 3.96-3.89 (m, 2H), 3.12-2.98 (m, 1H), 2.82-2.76 (m, 3H), 2.21-2.18 (m, 3H), 1.92-1.84 (m, 2H), 1.82-1.76 (m, 2H), 1.46-1.42 (m, 3H).

### Example 2: 5-(Azetidin-3-ylamino)-N-(1-(2,3-dihydrobenzo[b][1,4]dioxin-5-yl)ethyl)-2-methylbenzamide (Compound 168)

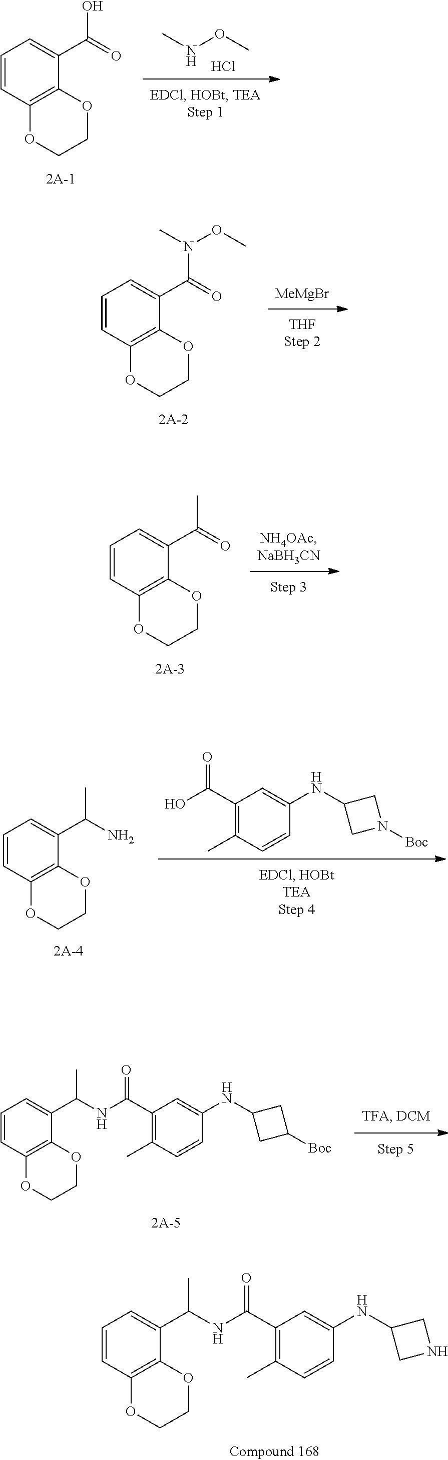

### Step 1: N-Methoxy-N-methyl-2,3-dihydrobenzo[b][1,4]dioxine-5-carboxamide (2A-2)

To a solution of 2,3-dihydro-1,4-benzodioxine-5-carboxylic acid (1.00 g, 5.55 mmol, 1.0 eq) and N,O)-dimethylhydroxylamine hydrochloride (596 mg, 6.11 mmol, 1.1 eq) in DCM (20 mL) were added TEA (1.69 g, 16.7 mmol, 2.32 mL, 3.0 eq), EDCI (1.28 g, 6.66 mmol, 1.2 eq) and HOBt (900 mg, 6.66 mmol, 1.2 eq). The mixture was stirred at 20° C. for 16 h. LCMS indicated that the starting material was completely consumed, and the desired mass was detected. The reaction mixture was poured into H₂O (20 mL) and extracted with DCM (10 mL×2). The combined organic layers were dried over Na₂SO₄, filtered, and concentrated under vacuum to give a residue which was purified by flash silica gel chromatography using a gradient of EtOAc/petroleum ether from 1/100 to 1/3. N-Methoxy-N-methyl-2,3-dihydrobenzo[b][1,4]dioxine-5-carboxamide (600 mg, 2.69 mmol, 48% yield) was obtained as a white solid. M+H⁺=224.0 (LCMS).

### Step 2: 1-(2,3-Dihydrobenzo[b][1,4]dioxin-5-yl)ethanone (2A-3)

A mixture of N-methoxy-N-methyl-2,3-dihydrobenzo[b][1,4]dioxine-5-carboxamide (200 mg, 896 μmol, 1.0 eq) in THF (3.0 mL) was degassed and purged with N₂ three times, and to this mixture was added MeMgBr (3 M in Et₂O, 388 μL, 1.3 eq) at 0° C. The mixture was warmed to 20° C. and stirred at the same temperature for 2 h under a N₂ atmosphere. LCMS indicated that the starting material was completely consumed, and the desired mass was detected. The reaction mixture was poured into H₂O (10 mL) and extracted with EtOAc (4.0 mL×3). The combined organic layers were dried over Na₂SO₄, filtered, and concentrated under vacuum to give the crude product 1-(2,3-dihydrobenzo[b][1,4]dioxin-5-yl)ethanone (150 mg), which was used in the next step without any further purification.

### Step 3: 1-(2,3-Dihydrobenzo[b][1,4]dioxin-5-yl)ethanamine (2A-4)

To a solution of 1-(2,3-dihydrobenzo[b][1,4]dioxin-5-yl)ethanone (100 mg, 561 μmol, 1.0 eq) in MeOH (1.0 mL) was added NH₄OAc (519 mg, 6.73 mmol, 12 eq), followed by NaBH₃CN (141 mg, 2.24 mmol, 4.0 eq) at 20° C. The mixture was stirred at the same temperature for 16 h. LCMS indicated that the starting material was completely consumed, and the desired compound was detected as a main peak. The reaction mixture was poured into H₂O (5.0 mL) and extracted with DCM (3.0 mL×2). The combined organic layers were dried over Na₂SO₄, filtered, and concentrated under vacuum to give the crude product 1-(2,3-dihydrobenzo[b][1,4]dioxin-5-yl)ethanamine (150 mg), which was used in the next step without any further purification. M+H⁺=180.0 (LCMS).

### Step 4: tert-Butyl-((3-((1-(2,3-dihydrobenzo[b][1,4]dioxin-5-yl)ethyl)carbamoyl)-4-methylphenyl)amino)azetidine-1-carboxylate (2A-5)

To a solution of 1-(2,3-dihydrobenzo[b][1,4]dioxin-5-yl)ethanamine (130 mg, 725 μmol, 1.1 eq) and 5-((1-(tert-butoxycarbonyl)azetidin-3-yl)amino)-2-methylbenzoic acid (202 mg, 659 μmol, 1.0 eq) in DCM (5.0 mL) were added TEA (267 mg, 2.64 mmol, 367 μL, 4.0 eq), EDCI (379 mg, 1.98 mmol, 3.0 eq) and HOBt (267 mg, 1.98 mmol, 3.0 eq). The mixture was stirred at 20° C. for 16 h. LCMS indicated that the starting material was completely consumed, and the desired mass was detected. The reaction mixture was poured into H₂O (5.0 mL) and extracted with DCM (3.0 mL×2). The combined organic layers were dried over Na₂SO₄, filtered, and concentrated under vacuum to give a residue which was purified by flash silica gel chromatography using a gradient of EtOAc/petroleum ether from 1/100 to 1/3. tert-Butyl-((3-((1-(2,3-dihydrobenzo[b][1,4]dioxin-5-yl)ethyl)carbamoyl)-4-methylphenyl)amino)azetidine-1-carboxylate (123 mg, 263 μmol, 40% yield) was obtained as a white oil. M+H⁺=468.0 (LCMS).

### Step 5: 5-(Azetidin-3-ylamino)-N-(1-(2,3-dihydrobenzo[b][1,4]dioxin-5-yl)ethyl)-2-methylbenzamide (Compound 168)

To a solution of tert-butyl 3-((3-((1-(2,3-dihydrobenzo[b][1,4]dioxin-5-yl)ethyl)carbamoyl)-4-methylphenyl)amino)azetidine-1-carboxylate (20.0 mg, 42.8 μmol, 1.0 eq) in DCM (3.0 mL) was added TFA (1.54 g, 13.5 mmol, 1.00 mL, 316 eq). The mixture was stirred at 20° C. for 16 h. LCMS indicated that the starting material was completely consumed, and the desired mass was detected. The mixture was concentrated under vacuum at 20° C. to give a residue which was purified by preparative HPLC (Phenomenex Luna C18 column (150×30 mm, 5 μm); flow rate: 25 mL/min; gradient: 10%-45% B over 8 min; mobile phase A: 0.1% aqueous TFA, mobile phase B: acetonitrile) to give 5-(azetidin-3-ylamino)-N-(1-(2,3-dihydrobenzo[b][1,4]dioxin-5-yl)ethyl)-2-methylbenzamide (12.0 mg, 24.6 μmol, 58% yield, TFA salt) as a white solid. M+H⁺=368.0 (LCMS); ¹H NMR (400 MHZ, CD₃OD) δ 8.54-8.47 (m, 1H), 7.04 (d, J=8.1 Hz, 1H), 6.88-6.86 (m, 1H), 6.81-6.73 (m, 2H), 6.59-6.54 (m, 2H), 5.48-5.40 (m, 1H), 4.53-4.45 (m, 1H), 4.39-4.30 (m, 4H), 4.27-4.24 (m, 2H), 3.96-3.91 (m, 2H), 2.22 (s, 3H), 1.46 (d, J=7.0 Hz, 3H).

### Example 3: (R)-5-Acetyl-2-methyl-N-(1-(naphthalen-1-yl)ethyl)benzamide (Compound 155)

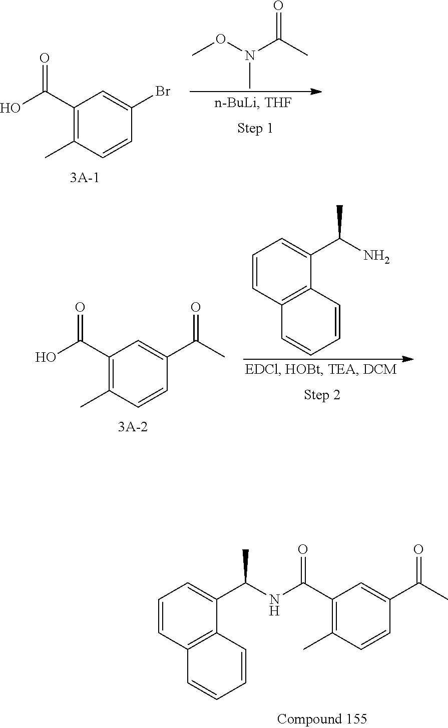

### Step 1: 5-Acetyl-2-methylbenzoic acid (3A-2)

To a solution of 5-bromo-2-methylbenzoic acid (300 mg, 1.40 mmol, 1.0 eq) in THF (10 mL) was added n-BuLi (2.5 M in hexane, 1.12 mL, 2.0 eq) at −78° C. The mixture was stirred at −78° C. for 30 min. To the mixture was added a solution of N-methoxy-N-methylacetamide (158 mg, 1.53 mmol, 163 μL, 1.1 eq) in THF (3.0 mL). The mixture was stirred at −78° C. for 1 h, then warmed to 20° C. and stirred at 20° C. for another 1 h. LCMS indicated that the starting material was completely consumed, and the desired mass was detected. The reaction mixture was poured into HCl (1 M aqueous, 10 mL) and extracted with MTBE (10 mL×2). The combined organic layers were washed with brine (10 mL×3), dried over Na₂SO₄, filtered, and concentrated under vacuum to give the crude product 5-acetyl-2-methylbenzoic acid (70.0 mg), which was used in the next step without any further purification. M+H⁺=179.1 (LCMS); 1H NMR (400 MHZ, DMSO-d₆) δ 13.1 (br d, J=5.4 Hz, 1H), 8.3 (d, J=2.0 Hz, 1H), 8.0 (dd, J=8.0, 2.0 Hz, 1H), 7.5 (d, J=8.0 Hz, 1H), 2.6 (s, 6H).

### Step 2: (R)-5-Acetyl-2-methyl-N-(1-(naphthalen-1-yl)ethyl)benzamide (Compound 155)

To a solution of 5-acetyl-2-methylbenzoic acid (60.0 mg, 337 μmol, 1.0 eq) in DCM (3.0 mL) were added (R)-1-(naphthalen-1-yl)ethanamine (57.7 mg, 337 μmol, 53.9 μL, 1.0 eq), EDCI (77.5 mg, 404 μmol, 1.2 eq), HOBt (54.6 mg, 404 μmol, 1.2 eq) and TEA (102 mg, 1.01 mmol, 141 μL, 3.0 eq). The mixture was stirred at 20° C. for 16 h. LCMS indicated that the starting material was completely consumed, and the desired mass was detected. The reaction mixture was poured into H₂O (10 mL) and extracted with EtOAc (5.0 mL×3). The combined organic layers were dried over Na₂SO₄, filtered, and concentrated under vacuum to give a residue which was purified by preparative HPLC (Phenomenex Luna C18 column (150×30 mm, 5 μm); flow rate: 25 mL/min; gradient: 35%-70% B over 8 min; mobile phase A: 0.2% aqueous FA, mobile phase B: acetonitrile). (R)-5-Acetyl-2-methyl-N-(1-(naphthalen-1-yl)ethyl)benzamide (11.5 mg, 34.5 μmol, 10% yield) was obtained as a white solid. M+H⁺=332.1 (LCMS); ¹H NMR (400 MHZ, CD₃OD) δ 8.3 (d, J=8.4 Hz, 1H), 7.9-8.0 (m, 3H), 7.8 (d, J=8.1 Hz, 1H), 7.6-7.7 (m, 2H), 7.5-7.5 (m, 2H), 7.4 (d, J=8.0 Hz, 1H), 6.1 (q, J=6.9 Hz, 1H), 2.5-2.6 (m, 3H), 2.4 (s, 3H), 1.7 (d, J=7.0 Hz, 3H).

### Example 4: (R)-5-Hydroxy-2-methyl-N-(1-(naphthalen-1-yl)ethyl)benzamide (Compound 101)

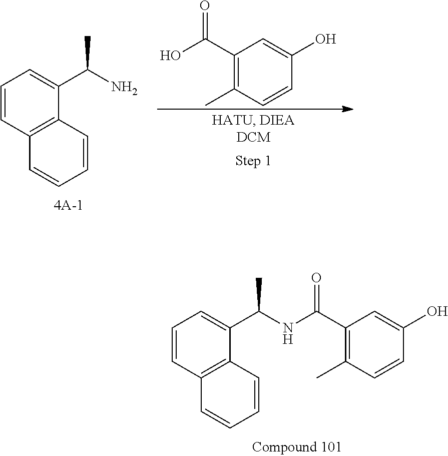 [SMILES: C[C@@H](N)c1cccc2ccccc12 ; Cc1ccc(O)cc1C(=O)O ; Cc1ccc(O)cc1C(=O)N[C@H](C)c1cccc2ccccc12]

### Step 1: (R)-5-Hydroxy-2-methyl-N-(1-(naphthalen-1-yl)ethyl)benzamide (Compound 101)

A mixture of (R)-1-(naphthalen-1-yl)ethanamine (56.3 mg, 329 μmol, 52.6 μL, 1.0 eq) and 5-hydroxy-2-methylbenzoic acid (50.0 mg, 329 μmol, 1.0 eq) in DCM (3.0 mL) were added HATU (250 mg, 657 μmol, 2.0 eq) and DIEA (127 mg, 986 μmol, 172 μL, 3.0 eq). The resulting mixture was stirred at 20° C. for 3 h. LCMS indicated that the starting material was completely consumed, and the desired mass was detected. The reaction mixture was poured into H₂O (5.0 mL) and extracted with DCM (2.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 30%-60% B over 8 min; mobile phase A: water (10 mM NH₄HCO₃), mobile phase B: acetonitrile) to give (R)-5-hydroxy-2-methyl-N-(1-(naphthalen-1-yl)ethyl)benzamide (50.2 mg, 164 μmol, 50% yield) as a white solid. M+H⁺=306.2 (LCMS); ¹H NMR (400 MHZ, CDCl₃) δ 8.19 (br d, J=8.3 Hz, 1H), 7.91-7.76 (m, 2H), 7.60-7.40 (m, 4H), 6.97 (d, J=8.3 Hz, 1H), 6.78 (s, 1H), 6.73 (br d, J=8.3 Hz, 1H), 6.09 (br s, 2H), 2.29 (s, 3H), 1.76 (br d, J=5.7 Hz, 3H).

### Example 5: (R)-5-Methoxy-2-methyl-N-(1-(naphthalen-1-yl)ethyl)benzamide (Compound 136)

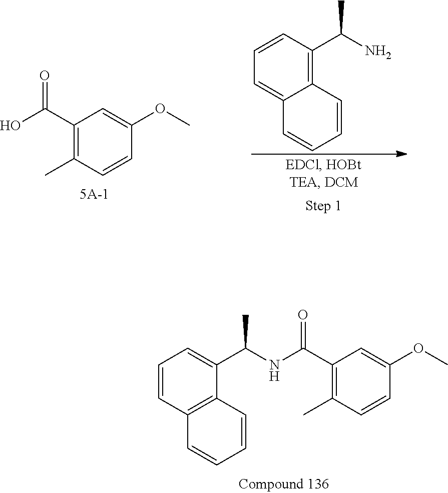 [SMILES: COc1ccc(C)c(C(=O)O)c1 ; C[C@@H](N)c1cccc2ccccc12 ; COc1ccc(C)c(C(=O)N[C@H](C)c2cccc3ccccc23)c1]

### Step 1: (R)-5-Methoxy-2-methyl-N-(1-(naphthalen-1-yl)ethyl)benzamide (Compound 136)

To a mixture of (R)-1-(naphthalen-1-yl)ethanamine (50 mg, 292 μmol, 47.0 μL, 1.0 eq) and 5-methoxy-2-methylbenzoic acid (48.6 mg, 292 μmol, 1.0 eq) in DCM (2.0 mL) were added EDCI (67.2 mg, 350 μmol, 1.2 eq), HOBt (47.4 mg, 350 μmol, 1.2 eq) and TEA (88.6 mg, 876 μmol, 100 μL, 3.0 eq). The resulting mixture was stirred at 20° C. for 3 h. LCMS indicated that the starting material was completely consumed, and the desired mass was detected. The reaction mixture was poured into H₂O (5.0 mL) and extracted with DCM (2.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 45%-75% B over 8 min; mobile phase A: water (10 mM NH₄HCO₃), mobile phase B: acetonitrile). (R)-5-Methoxy-2-methyl-N-(1-(naphthalen-1-yl)ethyl)benzamide (36.1 mg, 112 μmol, 38% yield) was obtained as a white solid. M+H⁺=320.1 (LCMS); ¹H NMR (400 MHZ, CDCl₃) δ 8.24 (d, J=8.4 Hz, 1H), 7.89 (d, J=7.7 Hz, 1H), 7.83 (d, J=8.2 Hz, 1H), 7.63-7.43 (m, 4H), 7.09 (d, J=8.3 Hz, 1H), 6.87-6.78 (m, 2H), 6.19-6.08 (m, 1H), 3.74 (s, 3H), 2.35 (s, 3H), 1.80 (d, J=6.7 Hz, 3H).

### Example 6: (R)-2-Chloro-5-methoxy-N-(1-(naphthalen-1-yl)ethyl)benzamide (Compound 132)

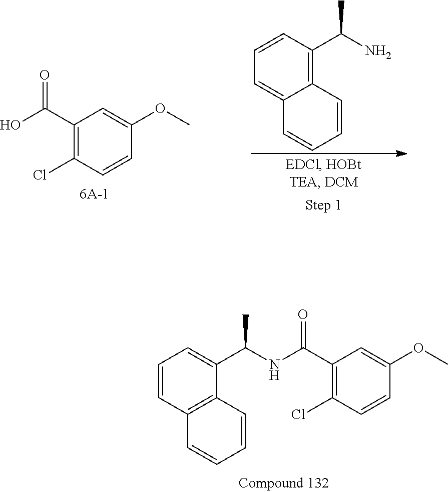 [SMILES: COc1ccc(Cl)c(C(=O)O)c1 ; C[C@@H](N)c1cccc2ccccc12 ; COc1ccc(Cl)c(C(=O)N[C@H](C)c2cccc3ccccc23)c1]

### Step 1: (R)-2-Chloro-5-methoxy-N-(1-(naphthalen-1-yl)ethyl)benzamide (Compound 132)

To a mixture of (R)-1-(naphthalen-1-yl)ethanamine (60.0 mg, 350 μmol, 56.0 μL, 1.0 eq) and 2-chloro-5-methoxybenzoic acid (65.4 mg, 350 μmol, 1.0 eq) in DCM (3.0 mL) were added EDCI (80.6 mg, 420 μmol, 1.2 eq) and HOBt (56.8 mg, 420 μmol, 1.2 eq) and TEA (106 mg, 1.05 mmol, 146 μL, 3.0 eq). The resulting mixture was stirred at 20° C. for 3 h. LCMS indicated that the starting material was completely consumed, and the desired mass was detected. The reaction mixture was poured into H₂O (5.0 mL) and extracted with DCM (2.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 50%-80% B over 8 min; mobile phase A: water (10 mM NH₄HCO₃), mobile phase B: acetonitrile). (R)-2-Chloro-5-methoxy-N-(1-(naphthalen-1-yl)ethyl)benzamide (56.3 mg, 161 μmol, 46% yield) was obtained as a white solid. M+H⁺=340.0 (LCMS); ¹H NMR (400 MHZ, CDCl₃) δ 8.23 (d, J=8.5 Hz, 1H), 7.89 (d, J=7.7 Hz, 1H), 7.82 (d, J=8.0 Hz, 1H), 7.63-7.42 (m, 4H), 7.25 (d, J=8.9 Hz, 1H), 7.22 (d, J=3.0 Hz, 1H), 6.88 (dd, J=3.1, 8.8 Hz, 1H), 6.14 (q, J=6.9 Hz, 1H), 3.79 (s, 3H), 1.82 (d, J=6.8 Hz, 3H).

### Example 7: (R)-tert-Butyl(2-(4-methyl-3-((1-(naphthalen-1-yl)ethyl)carbamoyl)phenoxy)ethyl)carbamate (Compound 158)

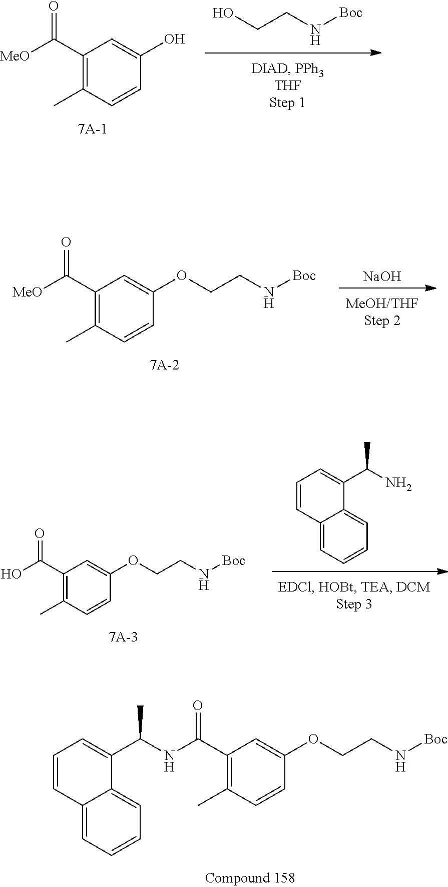

### Step 1: Methyl 5-(2-((tert-butoxycarbonyl)amino)ethoxy)-2-methylbenzoate (7A-2)

A mixture of tert-butyl(2-hydroxyethyl)carbamate (2.43 g, 15.0 mmol, 2.33 mL, 1.0 eq), methyl 5-hydroxy-2-methylbenzoate (2.50 g, 15.0 mmol, 1.0 eq) and PPh₃ (4.34 g, 16.6 mmol, 1.1 eq) in THF (30 mL) was degassed and purged with N₂ three times. To the mixture was added DIAD (3.35 g, 16.6 mmol, 3.22 mL, 1.1 eq) dropwise at 20° C. The resulting mixture was stirred at 70° C. for 16 h under a N₂ atmosphere. LCMS indicated that the starting material was completely consumed, and the desired mass was detected. The reaction mixture was allowed to cool to room temperature, poured into H₂O (30 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/100 to 1/2. Methyl 5-(2-((tert-butoxycarbonyl)amino)ethoxy)-2-methylbenzoate (2.50 g, 8.08 mmol, 54% yield) was obtained as a yellow oil. M+Na⁺=332.0 (LCMS); ¹H NMR (400 MHZ, CDCl₃) δ 7.43 (d, J=2.8 Hz, 1H), 7.14 (d, J=8.4 Hz, 1H), 6.94 (dd, J=2.8, 8.3 Hz, 1H), 5.11-4.95 (m, 1H), 4.02 (s, 2H), 3.88 (s, 3H), 3.53 (br d, J=5.1 Hz, 2H), 2.51 (s, 3H), 1.45 (s, 9H).

### Step 2: 5-(2-((tert-Butoxycarbonyl)amino)ethoxy)-2-methylbenzoic acid (7A-3)

To a solution of methyl 5-(2-((tert-butoxycarbonyl)amino)ethoxy)-2-methylbenzoate (1.60 g, 5.17 mmol, 1.0 eq) in a mixture of MeOH (8.0 mL) and THF (24 mL) was added NaOH (2 M aqueous, 10 mL, 4.0 eq). The mixture was stirred at 70° C. for 8 h. LCMS indicated that the starting material was completely consumed, and the desired mass was detected. The reaction mixture was allowed to cool to room temperature, H₂O (30 mL) was added, and the mixture was washed with MTBE (15 mL×2). The aqueous layer was acidified to pH 5 with HCl (1 M aqueous). The product was extracted with EtOAc (8.0 mL×3). The combined organic layers were dried over Na₂SO₄, filtered, and concentrated under vacuum to give the crude product 5-(2-((tert-butoxycarbonyl)amino)ethoxy)-2-methyl benzoic acid (1.1 g), which was used in the next step without any further purification. M+Na⁺=318.2 (LCMS); ¹H NMR (400 MHZ, DMSO-d₆) δ 13.49 (br s, 1H), 9.33 (s, 1H), 8.45 (d, J=7.5 Hz, 1H), 7.28 (br s, 1H), 7.00-6.92 (m, 1H), 6.70 (t, J=8.4 Hz, 2H), 5.18-5.11 (m, 1H), 2.64-2.54 (m, 2H), 2.00-1.90 (m, 1H), 1.87-1.72 (m, 3H).

### Step 3: (R)-tert-Butyl(2-(4-methyl-3-((1-(naphthalen-1-yl)ethyl)carbamoyl)phenoxy)ethyl)carbamate (Compound 158)

To a solution of 5-(2-((tert-butoxycarbonyl)amino)ethoxy)-2-methylbenzoic acid (50.0 mg, 169 μmol, 1.0 eq) and (R)-1-(naphthalen-1-yl)ethanamine (34.8 mg, 203 μmol, 32.5 μL, 1.2 eq) in DCM (3.0 mL) were added TEA (51.4 mg, 508 μmol, 70.7 μL, 3.0 eq), EDCI (48.7 mg, 254 μmol, 1.5 eq) and HOBt (34.3 mg, 254 μmol, 1.5 eq). The mixture was stirred at 20° C. for 16 h. TLC indicated that the starting material was completely consumed. The reaction mixture was poured into H₂O (3.0 mL) and extracted with EtOAc (3.0 mL×3). The combined organic layers were washed with brine (3.0 mL×3), dried over Na₂SO₄, and concentrated under vacuum to give a residue which was purified by preparative TLC (EtOAc/petroleum ether=1/3, R_{f}=0.5). (R)-tert-Butyl(2-(4-methyl-3-((1-(naphthalen-1-yl)ethyl)carbamoyl) phenoxy)ethyl)carbamate (77.2 mg, 143 μmol, 84% yield) was obtained as a white oil. M−56+H⁺=393.0 (LCMS); ¹H NMR (400 MHZ, CDCl₃) δ 8.24 (d, J=8.6 Hz, 1H), 7.89 (d, J=7.8 Hz, 1H), 7.83 (d, J=8.3 Hz, 1H), 7.62-7.45 (m, 4H), 7.09 (d, J=8.4 Hz, 1H), 6.85-6.77 (m, 2H), 6.18-6.09 (m, 1H), 5.98-5.91 (m, 1H), 4.94 (br d, J=4.9 Hz, 1H), 3.98-3.90 (m, 2H), 3.52-3.43 (m, 2H), 2.35 (s, 3H), 1.81 (d, J=6.7 Hz, 3H), 1.44 (s, 9H).

### Example 8: (R)-5-(2-Aminoethoxy)-2-methyl-N-(1-(naphthalen-1-yl)ethyl)benzamide (Compound 159)

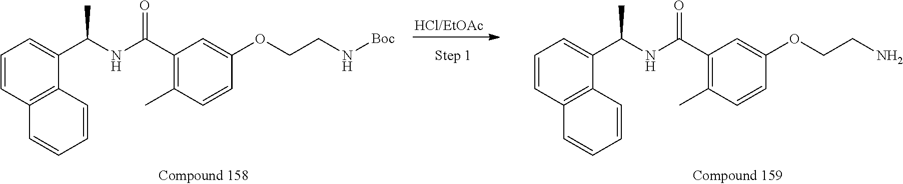 [SMILES: Cc1ccc(OCCNC(=O)OC(C)(C)C)cc1C(=O)N[C@H](C)c1cccc2ccccc12 ; Cc1ccc(OCCN)cc1C(=O)N[C@H](C)c1cccc2ccccc12]

### Step 1: (R)-5-(2-Aminoethoxy)-2-methyl-N-(1-(naphthalen-1-yl)ethyl)benzamide (Compound 159)

To a mixture of (R)-tert-butyl(2-(4-methyl-3-((1-(naphthalen-1-yl)ethyl) carbamoyl)phenoxy)ethyl)carbamate (20.0 mg, 44.6 μmol, 1.0 eq) in EtOAc (2.0 mL) was added HCl/EtOAc (4 M, 2.0 mL). The mixture was stirred at 20° C. for 30 min. LCMS indicated that the starting material was completely consumed, and the desired mass was detected. The reaction mixture was concentrated under vacuum to give a residue which was purified by preparative HPLC (Phenomenex Luna column (80×30 mm, 3 μm); flow rate: 25 mL/min; gradient: 10%-45% B over 8 min; mobile phase A: 0.04% aqueous HCl, mobile phase B: acetonitrile). (R)-5-(2-Aminoethoxy)-2-methyl-N-(1-(naphthalen-1-yl)ethyl)benzamide (6.21 mg, 16.1 μmol, 36% yield, HCl salt) was obtained as a white solid. M+H⁺=349.1 (LCMS); ¹H NMR (400 MHZ, CD₃OD) δ 8.26 (d, J=8.4 Hz, 1H), 7.91 (d, J=7.8 Hz, 1H), 7.82 (d, J=8.3 Hz, 1H), 7.70-7.44 (m, 4H), 7.17 (d, J=8.5 Hz, 1H), 6.97 (dd, J=2.8, 8.4 Hz, 1H), 6.92 (d, J=2.6 Hz, 1H), 6.06 (q, J=7.0 Hz, 1H), 4.59 (br s, 2H), 4.28-4.11 (m, 2H), 3.33 (d, J=5.1 Hz, 2H), 2.27 (s, 3H), 1.71 (d, J=6.9 Hz, 3H).

### Example 9: (R)-5-(2-(Dimethylamino)ethoxy)-2-methyl-N-(1-(naphthalen-1-yl)ethyl)benzamide (Compound 160)

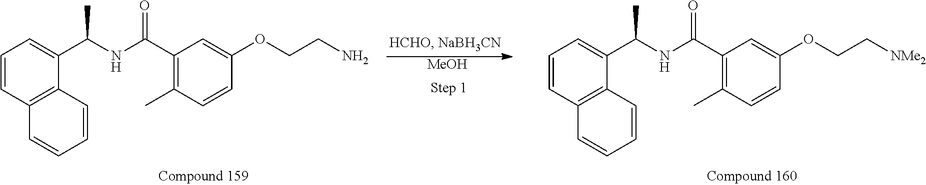 [SMILES: "MeOH" ; Cc1ccc(OCCN)cc1C(=O)N[C@H](C)c1cccc2ccccc12 ; Cc1ccc(OCCN(C)C)cc1C(=O)N[C@H](C)c1cccc2ccccc12]

### Step 1: (R)-5-(2-(Dimethylamino)ethoxy)-2-methyl-N-(1-(naphthalen-1-yl)ethyl)benzamide (Compound 160)

To a solution of (R)-5-(2-aminoethoxy)-2-methyl-N-(1-(naphthalen-1-yl)ethyl)benzamide (20.0 mg, 57.4 μmol, 1.0 eq, HCl salt) in MeOH (3.0 mL) was added TEA (900 μL), followed by the addition of formaldehyde (9.32 mg, 115 μmol, 8.55 μL, 37% purity in water, 2.0 eq). The resulting mixture was adjusted to pH 6 with a small amount of AcOH. The mixture was stirred at 20° C. for 30 min, then NaBH₃CN (7.21 mg, 115 μmol, 2.0 eq) was added. The resulting mixture was stirred at 20° C. for another 16 h. LCMS indicated that the starting material was completely consumed, and the desired mass was detected. The reaction mixture was poured into H₂O (3.0 mL) at 25° C. and extracted with EtOAc (3.0 mL×3). The combined organic layers were washed with brine (3.0 mL×3), dried over Na₂SO₄, and concentrated under vacuum to give a residue which was purified by preparative HPLC (Phenomenex Luna column (80×30 mm, 3 μm); flow rate: 25 mL/min; gradient: 5%-50% B over 8 min; mobile phase A: 0.04% aqueous HCl, mobile phase B: acetonitrile). (R)-5-(2-(Dimethylamino)ethoxy)-2-methyl-N-(1-(naphthalen-1-yl)ethyl)benzamide (15.9 mg, 42.2 μmol, 74% yield, HCl salt) was obtained as a white solid. M+H⁺=377.1 (LCMS); ¹H NMR (400 MHZ, CD₃OD) δ 8.85 (br d, J=7.8 Hz, 1H), 8.25 (d, J=8.3 Hz, 1H), 7.90 (d, J=8.3 Hz, 1H), 7.82 (d, J=8.3 Hz, 1H), 7.63 (d, J=6.8 Hz, 1H), 7.60-7.43 (m, 3H), 7.18 (d, J=8.4 Hz, 1H), 6.98 (dd, J=2.8, 8.4 Hz, 1H), 6.93 (d, J=2.7 Hz, 1H), 6.11-5.97 (m, 1H), 4.35-4.25 (m, 2H), 3.61-3.51 (m, 2H), 2.95 (s, 6H), 2.28 (s, 3H), 1.71 (d, J=6.8 Hz, 3H).

### Example 10: (R)-2-Methyl-N-(1-(naphthalen-1-yl)ethyl)-5-(oxetan-3-yloxy)benzamide (Compound 152)

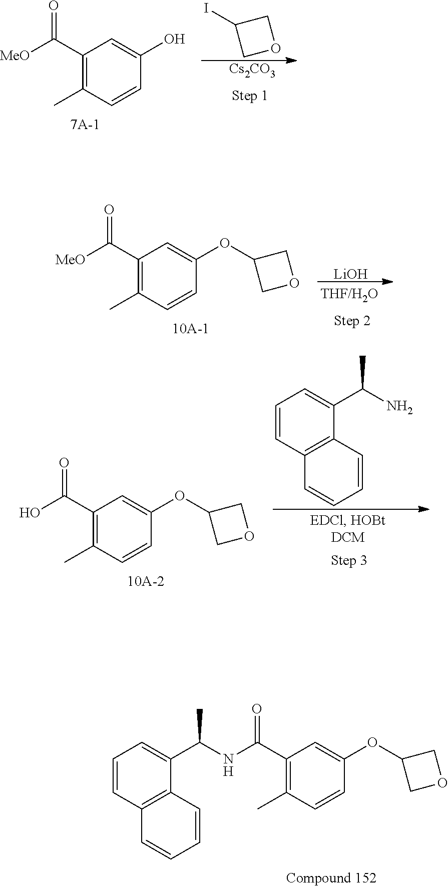

### Step 1: Methyl 2-methyl-5-(oxetan-3-yloxy)benzoate (10A-1)

To a solution of methyl 5-hydroxy-2-methylbenzoate (150 mg, 903 μmol, 1.0 eq) and 3-iodooxetane (199 mg, 1.08 mmol, 1.2 eq) in DMF (4 mL) was added Cs₂CO₃ (588 mg, 1.81 mmol, 2.0 eq). The mixture was stirred at 110° C. for 16 h. LCMS indicated that the starting material was completely consumed, and the desired mass was detected. The reaction mixture was allowed to cool to room temperature, poured into H₂O (5.0 mL) and extracted with EtOAc (2.0 mL×3). The combined organic layers were dried over Na₂SO₄, filtered, and concentrated under vacuum to give methyl 2-methyl-5-(oxetan-3-yloxy)benzoate (1.10 g), which was used in the next step without any further purification.

### Step 2: 2-Methyl-5-(oxetan-3-yloxy)benzoic acid (10A-2)

To a solution of methyl 2-methyl-5-(oxetan-3-yloxy)benzoate (80.0 mg, 360 μmol, 1.0 eq) in a mixture of THF (3.0 mL) and H₂O (1.0 mL) was added LiOH·H₂O (45.3 mg, 1.08 mmol, 3.0 eq). The mixture was stirred at 20° C. for 16 h. LCMS indicated that the starting material completely consumed, and desired mass was detected. The reaction mixture was poured into H₂O (3.0 mL) and extracted with petroleum ether (3.0 mL×2). The aqueous phase was adjusted to pH 4 with HCl (1 M aqueous) and extracted with EtOAc (2.0 mL×3). The combined organic layers were dried over Na₂SO₄, filtered, and concentrated under vacuum to give the crude product 2-methyl-5-(oxetan-3-yloxy)benzoic acid (60.0 mg) as a colorless oil, which was used in the next step without any further purification. M+H⁺=209.2 (LCMS).

### Step 3: (R)-2-methyl-N-(1-(naphthalen-1-yl)ethyl)-5-(oxetan-3-yloxy)benzamide (Compound 152)

To a solution of 2-methyl-5-(oxetan-3-yloxy)benzoic acid (50.0 mg, 240 μmol, 1.0 eq) and (R)-1-(naphthalen-1-yl)ethanamine (41.1 mg, 240 μmol, 38.4 μL, 1.0 eq) in DCM (3.0 mL) were added TEA (48.6 mg, 480 μmol, 2.0 eq), EDCI (55.2 mg, 288 μmol, 1.2 eq) and HOBt (38.9 mg, 288 μmol, 1.2 eq). The mixture was stirred at 20° C. for 16 h. LCMS indicated that the starting material was completely consumed, and desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue. The residue was purified by preparative TLC (EtOAc/petroleum ether=2/1) to give (R)-2-methyl-N-(1-(naphthalen-1-yl)ethyl)-5-(oxetan-3-yloxy)benzamide (12.2 mg, 33.2 μmol) as a white solid. M+H⁺=362.1 (LCMS); ¹H NMR (400 MHZ, CDCl₃) δ 8.22 (d, J=8.4 Hz, 1H), 7.90 (d, J=7.6 Hz, 1H), 7.84 (d, J=8.1 Hz, 1H), 7.66-7.43 (m, 4H), 7.10-7.04 (m, 1H), 6.66-6.62 (m, 1H), 6.62-6.56 (m, 1H), 6.18-6.06 (m, 1H), 5.98-5.88 (m, 1H), 5.16-5.05 (m, 1H), 4.93-4.82 (m, 2H), 4.72-4.62 (m, 2H), 2.40-2.31 (m, 3H), 1.84-1.75 (m, 3H).

### Example 11: (R)-5-(Azetidin-3-yloxy)-2-methyl-N-(1-(naphthalen-1-yl)ethyl)benzamide (Compound 141)

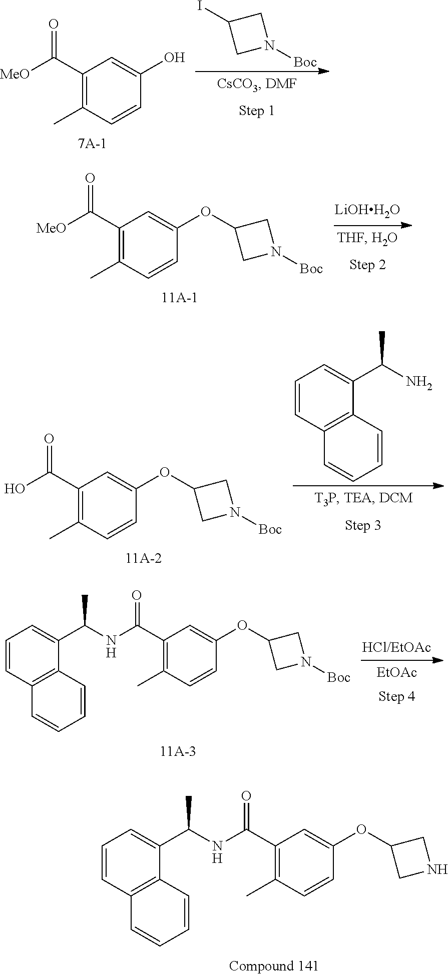

### Step 1: tert-Butyl 3-(3-(methoxycarbonyl)-4-methylphenoxy)azetidine-1-carboxylate (11A-1)

To a stirred solution of methyl 5-hydroxy-2-methylbenzoate (300 mg, 1.81 mmol, 1.0 eq) in DMF (10 mL) were added Cs₂CO₃ (1.18 g, 3.61 mmol, 2.0 eq) and tert-butyl 3-iodoazetidine-1-carboxylate (613 mg, 2.17 mmol, 1.2 eq). The mixture was stirred at 110° C. for 16 h. TLC indicated that the starting material was completely consumed. The reaction mixture was allowed to cool to room temperature, poured into water (30 mL) and extracted with EtOAc (10 mL×3). The combined organic layers were dried and concentrated under vacuum to give a residue which was purified by flash silica gel chromatography using a gradient of EtOAc/petroleum ether from 1/100 to 1/5. tert-Butyl 3-(3-(methoxycarbonyl)-4-methylphenoxy)azetidine-1-carboxylate (450 mg, 1.40 mmol, 78% yield) was obtained as a colorless oil. ¹H NMR (400 MHZ, CD₃Cl) δ 7.29 (s, 1H), 7.19-7.17 (d, J=8.4 Hz, 1H), 6.87-6.84 (m, 1H), 4.93-4.89 (m, 1H), 4.35-4.31 (m, 2H), 4.03-4.01 (m, 2H), 3.91 (s, 3H), 2.54 (s, 3H), 1.47 (s, 9H).

### Step 2: 5-((1-(tert-Butoxycarbonyl)azetidin-3-yl)oxy)-2-methylbenzoic acid (11A-2)

To a stirred solution of tert-butyl 3-(3-(methoxycarbonyl)-4-methylphenoxy)azetidine-1-carboxylate (200 mg, 622 μmol, 1.0 eq) in a mixture of THF (5.0 mL) and H₂O (1.0 mL) was added LiOH·H₂O (52.2 mg, 1.24 mmol, 2.0 eq) at 20° C. for 2 h then at 70° C. for another 14 h. Another 10.0 mg of LiOH·H₂O was added and the reaction mixture was stirred at 70° C. for another 1 h. LCMS indicated that the starting material was completely consumed. The reaction mixture was allowed to cool to room temperature, poured into H₂O (3.0 mL) and washed with TBME (5.0 mL×2). The aqueous layer was acidified to pH 5 with HCl (1 M aqueous). The product was extracted with DCM (3.0 mL×5) and the combined organic layers were dried over Na₂SO₄, filtered, and concentrated under vacuum to give the product 5-((1-(tert-butoxy carbonyl)azetidin-3-yl)oxy)-2-methylbenzoic acid (150 mg, 488 μmol, 78% yield) as a colorless oil, which was used in the next step without any further purification. M−H⁻=306.1 (LCMS).

### Step 3: (R)-tert-Butyl 3-(4-methyl-3-((1-(naphthalen-1-yl)ethyl)carbamoyl)phenoxy)azetidine-1-carboxylate (11A-3)

To a mixture of 5-(1-tert-butoxycarbonylazetidin-3-yl)oxy-2-methyl-benzoic acid (90.0 mg, 292 μmol, 1.0 eq) in DCM (5.0 mL) was added (1R)-1-(1-naphthyl)ethanamine (55.2 mg, 322 μmol, 51.6 μL, 1.1 eq), followed by TEA (88.9 mg, 879 μmol, 122 μL, 3.0 eq) and T₃P (280 mg, 439 μmol, 261 μL, 50% in EtOAc, 1.5 eq). The resulting mixture was stirred at 20° C. for 1.5 h. LCMS indicated that the starting material was completely consumed, and the desired mass was detected. The mixture was poured into H₂O (3.0 mL). The product was extracted with DCM (8.0 mL×5). The combined organic layers were dried over Na₂SO₄, filtered, and concentrated under vacuum to give a residue which was purified by flash silica gel chromatography using a gradient of EtOAc/petroleum ether from 1/100 to 1/3. (R)-tert-Butyl 3-(4-methyl-3-((1-(naphthalen-1-yl)ethyl)carbamoyl)phenoxy)azetidine-1-carboxylate (120 mg, 261 μmol, 89% yield) was obtained as a colorless oil. M+H⁺=461.4 (LCMS).

### Step 4: (R)-tert-Butyl 3-(4-methyl-3-((1-(naphthalen-1-yl)ethyl)carbamoyl)phenoxy)azetidine-1-carboxylate (Compound 141)

To a mixture of (R)-tert-butyl 3-(4-methyl-3-((1-(naphthalen-1-yl)ethyl)carbamoyl)phenoxy) azetidine-1-carboxylate (50.0 mg, 109 μmol, 1.0 eq) in EtOAc (5.0 mL) was added HCl/EtOAc (4 M, 10 mL) at 0° C. The mixture was stirred at 25° C. for 1 h. LCMS indicated that the starting material was completely consumed, and the desired mass was detected. The reaction mixture was concentrated under vacuum at 30° C. to give a residue which was purified by preparative HPLC (Phenomenex Gemini C18 column (75×30 mm, 3 μm); flow rate: 25 mL/min; gradient: 10%-40% B over 8 min; mobile phase A: 0.04% aqueous HCl, mobile phase B: acetonitrile). (R)-tert-Butyl3-(4-methyl-3-((1-(naphthalen-1-yl)ethyl)carbamoyl)phenoxy)azetidine-1-carboxylate (14.0 mg, 38.8 μmol, 36% yield) was obtained as a white solid. M+H⁺=361.1 (LCMS); ¹H NMR (400 MHZ, CD₃OD) δ 8.27-8.21 (m, 1H), 7.94-7.89 (m, 1H), 7.85-7.78 (m, 1H), 7.64-7.45 (m, 4H), 7.20-7.16 (m, 1H), 6.84-6.79 (m, 1H), 6.77-6.71 (m, 1H), 6.10-6.00 (m, 1H), 5.15-5.06 (m, 1H), 4.55-4.43 (m, 2H), 4.13-4.04 (m, 2H), 2.31-2.20 (m, 3H), 1.76-1.65 (m, 3H).

### Example 12: (R)-tert-Butyl 4-(4-methyl-3-((1-(naphthalen-1-yl)ethyl)carbamoyl)phenoxy) piperidine-1-carboxylate (Compound 164)

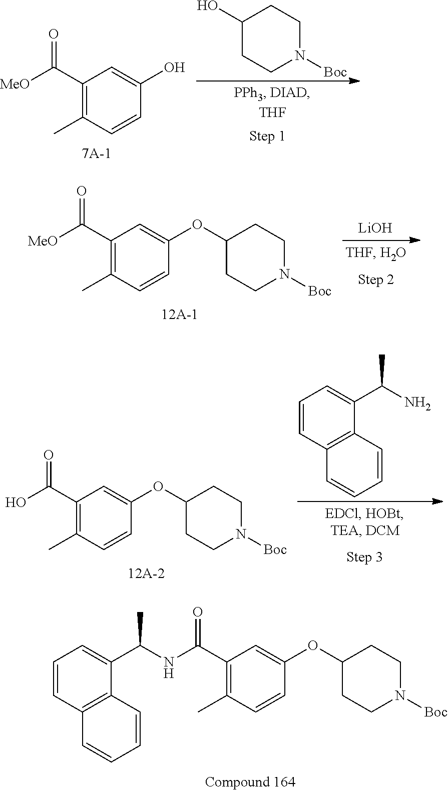

### Step 1: tert-Butyl 4-(3-(methoxycarbonyl)-4-methylphenoxy) piperidine-1-carboxylate (12A-1)

To a solution of methyl 5-hydroxy-2-methylbenzoate (200 mg, 1.20 mmol, 1.0 eq) in THF (20 mL) were added tert-butyl 4-hydroxypiperidine-1-carboxylate (266 mg, 1.32 mmol, 1.1 eq) and PPh₃ (379 mg, 1.44 mmol, 1.2 eq), followed by DIAD (316 mg, 1.56 mmol, 304 μL, 1.3 eq) at 0° C. under a N₂ atomsphere. The resulting mixture was stirred at 20° C. for 16 h. TLC indicated that the starting material was completely consumed. The mixture was poured into H₂O (10 mL) and extracted with DCM (10 mL×5). The combined organic layers were dried over Na₂SO₄, filtered, and concentrated under vacuum to give a residue which was purified by flash silica gel chromatography using a gradient of EtOAc/petroleum ether from 0/1 to 1/2. tert-Butyl 4-(3-(methoxycarbonyl)-4-methyl phenoxy) piperidine-1-carboxylate (200 mg, 572 μmol, 48% yield) was obtained as a yellow oil. ¹H NMR (400 MHZ, CDCl₃) δ 7.48-7.45 (m, 1H), 7.18-7.12 (m, 1H), 7.00-6.95 (m, 1H), 4.50-4.44 (m, 1H), 3.92-3.87 (m, 3H), 3.74-3.65 (m, 2H), 3.40-3.31 (m, 2H), 2.55-2.50 (m, 3H), 1.96-1.86 (m, 2H), 1.80-1.69 (m, 2H), 1.51-1.46 (m, 9H).

### Step 2: 5-((1-(tert-Butoxycarbonyl) piperidin-4-yl)oxy)-2-methylbenzoic acid (12A-2)

To a stirred solution of tert-butyl 4-(3-methoxycarbonyl-4-methyl-phenoxy) piperidine-1-carboxylate (120 mg, 343 μmol, 1.0 eq) in a mixture of THF (6.0 mL) and H₂O (2.0 mL) was added LiOH·H₂O (36.0 mg, 859 μmol, 2.5 eq) at 20° C. for 8 h and then at 70° C. for another 2 h. LCMS indicated that the starting material was completely consumed, and the desired mass was detected. The reaction mixture was allowed to cool to room temperature, poured into H₂O (10 mL) and washed with MTBE (5.0 mL×3). The aqueous was acidified to pH 5 with HCl (1 M aqueous) and the product was extracted with DCM (5.0 mL×8). The combined organic layers were dried over Na₂SO₄, filtered, and concentrated under vacuum to give the crude product 5-((1-(tert-butoxycarbonyl) piperidin-4-yl)oxy)-2-methylbenzoic acid (85.0 mg) as a colorless oil, which was used in the next step without any further purification. M−H⁻=334.2 (LCMS).

### Step 3: (R)-tert-Butyl 4-(4-methyl-3-((1-(naphthalen-1-yl)ethyl)carbamoyl)phenoxy)piperidine-1-carboxylate (Compound 164)

To a suspension of (1R)-1-(1-naphthyl)ethanamine (65.1 mg, 380 μmol, 60.8 μL, 1.5 eq) in DCM (5.0 mL) were added 5-[(1-tert-butoxycarbonyl-4-piperidyl)oxy]-2-methyl-benzoic acid (85.0 mg, 253 μmol, 1.0 eq) and TEA (76.9 mg, 760 μmol, 106 μL, 3.0 eq), followed by EDCI (72.9 mg, 380 μmol, 1.5 eq) and HOBt (51.4 mg, 380 μmol, 1.5 eq). The mixture was stirred at 20° C. for 16 h. LCMS indicated that the starting material was completely consumed, and the desired mass was detected. The mixture was poured into H₂O (10 mL) and extracted with DCM (5.0 mL×8). The combined organic layers were dried over Na₂SO₄, filtered, and concentrated under vacuum to give a residue which was purified by preparative TLC (EtOAc/petroleum ether=1/2, R_{f}=0.5). (R)-tert-Butyl 4-(4-methyl-3-((1-(naphthalen-1-yl)ethyl)carbamoyl)phenoxy) piperidine-1-carboxylate (110 mg, 218 μmol, 86% yield) was obtained as a white solid. M−56+H⁺=433.1; 1H NMR (400 MHZ, CDCl₃) δ 8.26-8.22 (m, 1H), 7.91-7.88 (m, 1H), 7.86-7.81 (m, 1H), 7.60 (s, 5H), 7.10-7.06 (m, 1H), 6.85 (s, 1H), 4.39-4.33 (m, 1H), 3.71-3.62 (m, 2H), 3.31-3.23 (m, 2H), 2.36-2.33 (m, 3H), 1.83-1.78 (m, 4H), 1.47-1.46 (m, 9H), 1.31-1.20 (m, 3H).

### Example 13: (R)-2-Methyl-N-(1-(naphthalen-1-yl)ethyl)-5-(piperidin-4-yloxy)benzamide (Compound 162)

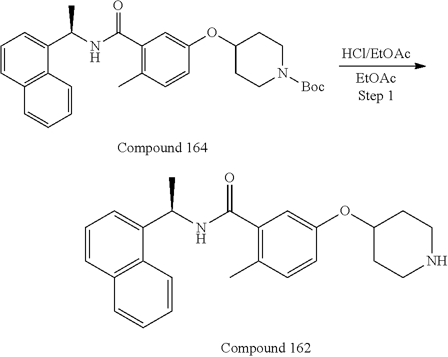 [SMILES: Cc1ccc(OC2CCN(C(=O)OC(C)(C)C)CC2)cc1C(=O)N[C@H](C)c1cccc2ccccc12 ; Cc1ccc(OC2CCNCC2)cc1C(=O)N[C@H](C)c1cccc2ccccc12]

### Step 1: (R)-2-Methyl-N-(1-(naphthalen-1-yl)ethyl)-5-(piperidin-4-yloxy)benzamide (Compound 162)

To a mixture of (R)-tert-butyl 4-(4-methyl-3-((1-(naphthalen-1-yl)ethyl)carbamoyl)phenoxy) piperidine-1-carboxylate (80.0 mg, 164 μmol, 1.0 eq) in EtOAc (3.0 mL) was added HCl/EtOAc (4 M, 40.9 μL). The resulting mixture was stirred at 20° C. for 1 h. LCMS indicated that the starting material was completely consumed, and the desired mass was detected. The reaction mixture was concentrated at 30° C. under vacuum to give a residue which was purified by preparative HPLC (Phenomenex Gemini C18 column (75×30 mm, 3 μm); flow rate: 25 mL/min; gradient: 10%-40% B over 8 min; mobile phase A: 0.04% aqueous HCl, mobile phase B: acetonitrile). (R)-2-Methyl-N-(1-(naphthalen-1-yl)ethyl)-5-(piperidin-4-yloxy)benzamide (40.0 mg, 103 μmol, 63% yield, HCl salt) was obtained as a white solid. M+H⁺=389.1; 1H NMR (400 MHZ, CD₃OD) δ 8.29-8.13 (m, 1H), 7.92-7.83 (m, 1H), 7.82-7.73 (m, 1H), 7.62-7.57 (m, 1H), 7.57-7.53 (m, 1H), 7.53-7.49 (m, 1H), 7.49-7.45 (m, 1H), 7.45-7.42 (m, 1H), 7.15-7.09 (m, 1H), 6.97-6.90 (m, 1H), 6.87-6.84 (m, 1H), 6.04-5.94 (m, 1H), 4.64-4.56 (m, 1H), 3.29 (br d, J=3.2 Hz, 2H), 3.19-3.09 (m, 2H), 2.24-2.20 (m, 3H), 2.14-2.01 (m, 2H), 2.01-1.83 (m, 2H), 1.69-1.62 (m, 3H).

### Example 14: (R)-2-Methyl-5-(methylsulfonamido)-N-(1-(naphthalen-1-yl)ethyl)benzamide (Compound 100)

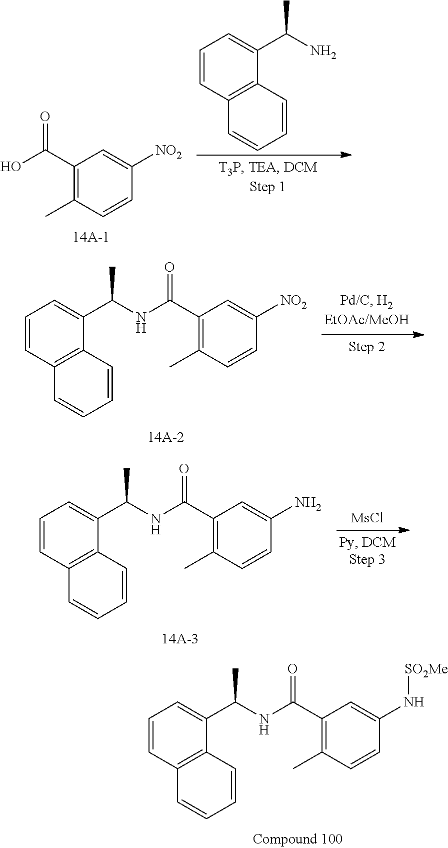

### Step 1: (R)-2-Methyl-N-(1-(naphthalen-1-yl)ethyl)-5-nitrobenzamide (14A-2)

To a solution of 2-methyl-5-nitrobenzoic acid (1.00 g, 5.52 mmol, 1.0 eq) and (R)-1-(naphthalen-1-yl)ethanamine (940 mg, 5.52 mmol, 880 μL, 1.0 eq) in DCM (20 mL) were added TEA (1.12 g, 11.0 mmol, 1.54 mL, 2.0 eq) and T₃P (10.5 g, 16.6 mmol, 10.0 mL, 50% in EtOAc, 3.0 eq) at 0° C. The resulting mixture was stirred at 20° C. for 3 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. (R)-2-Methyl-N-(1-(naphthalen-1-yl)ethyl)-5-nitrobenzamide (1.50 g, 4.49 mmol, 81% yield) as a white solid. M+H⁺=335.2 (LCMS).

### Step 2: (R)-5-Amino-2-methyl-N-(1-(naphthalen-1-yl)ethyl)benzamide (14A-3)

To a solution of (R)-2-methyl-N-(1-(naphthalen-1-yl)ethyl)-5-nitrobenzamide (1.00 g, 2.99 mmol, 1.0 eq), 10% palladium on carbon (200 mg) in a mixture of EtOAc (20 mL) and MeOH (20 mL) was degassed and purged with H₂ for three times, then the mixture was stirred at 20° C. for 16 h under H₂ (15 psi) atmosphere. LCMS indicated that the starting material was completely consumed, and the desired mass was detected. The suspension was filtered through a pad of Celite, and the filter cake was washed with EtOAc (5.0 mL×3). The combined filtrates were concentrated under vacuum to give (R)-5-amino-2-methyl-N-(1-(naphthalen-1-yl)ethyl)benzamide (800 mg, 2.58 mmol, 86% yield) as a white solid. M+H⁺=305.2 (LCMS).

### Step 3: (R)-2-Methyl-5-(methylsulfonamido)-N-(1-(naphthalen-1-yl)ethyl)benzamide (Compound 100)

To a solution of (R)-5-amino-2-methyl-N-(1-(naphthalen-1-yl)ethyl)benzamide (100 mg, 329 μmol, 1.0 eq) in Py (2.0 mL) was added MsCl (41.4 mg, 361 μmol, 28 μL, 1.1 eq) at 0° C. The resulting mixture was stirred at 20° C. for 3 h. LCMS indicated that the starting material was completely consumed, and the desired mass was detected. The reaction mixture was poured into H₂O (5.0 mL) and extracted with DCM (2.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 30%-60% B over 8 min; mobile phase A: 10 mM aqueous NH₄HCO₃, mobile phase B: acetonitrile). (R)-2-Methyl-5-(methylsulfonamido)-N-(1-(naphthalen-1-yl)ethyl)benzamide (38.5 mg, 100 μmol, 30% yield) as a white solid. M+H⁺=383.1 (LCMS); ¹H NMR (400 MHZ, CDCl₃) δ 8.21 (d, J=8.5 Hz, 1H), 7.91-7.86 (m, 1H), 7.81 (d, J=8.3 Hz, 1H), 7.61-7.42 (m, 4H), 7.17-7.09 (m, 3H), 6.80 (br s, 1H), 6.17-6.06 (m, 2H), 2.88 (s, 3H), 2.37 (s, 3H), 1.83-1.73 (m, 3H)

### Example 15: (R)-5-((1H-Pyrazol-4-yl)amino)-2-methyl-N-(1-(naphthalen-1-yl)ethyl)benzamide (Compound 121)

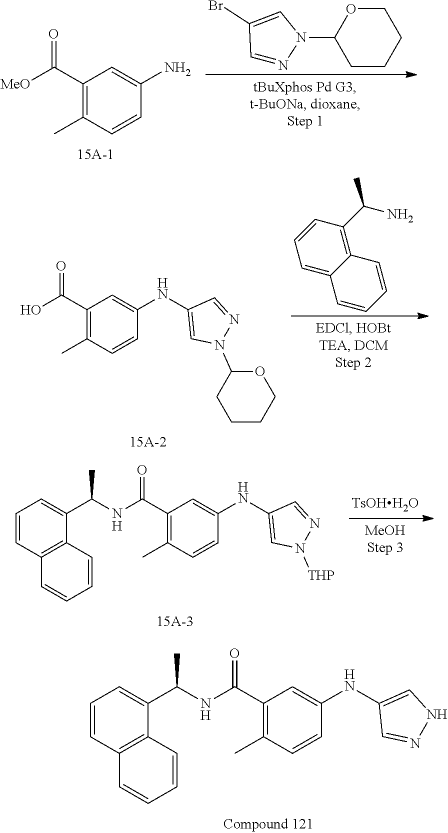

### Step 1: 2-Methyl-5-((1-(tetrahydro-2H-pyran-2-yl)-1H-pyrazol-4-yl)amino)benzoic acid (15A-2)

To a stirred solution of methyl 5-amino-2-methylbenzoate (50.0 mg, 303 μmol, 1.0 eq) and 4-bromo-1-(tetrahydro-2H-pyran-2-yl)-1H-pyrazole (83.9 mg, 363 μmol, 1.2 eq) in dioxane (2.0 mL) were added/BuXphos Pd G3 (24.0 mg, 30.3 μmol, 0.1 eq) and sodium 1-butanolate (145 mg, 1.51 mmol, 5.0 eq) under a N₂ atmosphere. The mixture was stirred at 100° C. for 16 h. LCMS indicated that the starting material was completely consumed, and the desired compound was detected. The reaction mixture was allowed to cool to room temperature, poured into H₂O (5.0 mL) and washed with MTBE (3.0 mL×2). The aqueous was acidified to pH 6 with HCl (1 M aqueous) and extracted with DCM (3.0 mL×3). The combined organic layers were dried over Na₂SO₄, filtered, and concentrated under vacuum to give 2-methyl-5-((1-(tetrahydro-2H-pyran-2-yl)-1H-pyrazol-4-yl)amino)benzoic acid (100 mg) as a yellow oil, which was used in the next step without any further purification. M+H⁺=302.3 (LCMS).

### Step 2: 2-Methyl-N—((R)-1-(naphthalen-1-yl)ethyl)-5-((1-(tetrahydro-2H-pyran-2-yl)-1H-pyrazol-4-yl)amino)benzamide (15A-3)

To a stirred solution of 2-methyl-5-((1-(tetrahydro-2H-pyran-2-yl)-1H-pyrazol-4-yl)amino)benzoic acid (130 mg, 431 μmol, 1.0 eq) and (R)-1-(naphthalen-1-yl)ethanamine (111 mg, 647 μmol, 104 μL, 1.5 eq) in DCM (5 mL) were added EDCI (124 mg, 647 μmol, 1.5 eq) and HOBt (87.4 mg, 647 μmol, 1.5 eq), followed by TEA (131 mg, 1.29 mmol, 180 μL, 3.0 eq). The mixture was stirred at 20° C. for 2 h. TLC indicated that the starting material was completely consumed. The mixture was poured into water (5.0 mL) and extracted with DCM (5.0 mL×2). The combined organic layers were dried over Na₂SO₄, filtered, and concentrated under vacuum to give a residue which was purified via preparative TLC (EtOAc/petroleum ether=1/2, R_{f}=0.2). 2-Methyl-N—((R)-1-(naphthalen-1-yl)ethyl)-5-((1-(tetrahydro-2H-pyran-2-yl)-1H-pyrazol-4-yl)amino)benzamide (90.0 mg, 198 μmol, 46% yield) was obtained as a yellow oil.

### Step 3: (R)-5-((1H-Pyrazol-4-yl)amino)-2-methyl-N-(1-(naphthalen-1-yl)ethyl)benzamide (Compound 121)

To a stirred solution of 2-methyl-N—((R)-1-(naphthalen-1-yl)ethyl)-5-((1-(tetrahydro-2H-pyran-2-yl)-1H-pyrazol-4-yl)amino)benzamide (90.0 mg, 198 μmol, 1.0 eq) in MeOH (2.0 mL) was added 4-methylbenzenesulfonic acid hydrate (113 mg, 594 μmol, 3.0 eq). The mixture was stirred at 20° C. for 2 h. LCMS indicated that the starting material was completely consumed, and the desired mass was detected. The mixture was poured into H₂O (3.0 mL) and extracted with EtOAc (3.0 mL×2). The combined organic layers were dried over Na₂SO₄, filtered, and concentrated under vacuum to give a residue which was purified by preparative HPLC (Phenomenex Gemini C18 column (150×40 mm, 10 μm); flow rate: 60 mL/min; gradient: 30%-60% B over 8 min; mobile phase A: 10 mM aqueous NH₄HCO₃, mobile phase B: acetonitrile). (R)-5-((1H-Pyrazol-4-yl)amino)-2-methyl-N-(1-(naphthalen-1-yl)ethyl)benzamide (30.0 mg, 80.7 μmol, 41% yield) was obtained as a white solid. M+H⁺=371.0 (LCMS); ¹H NMR (400 MHZ, DMSO-d₆) δ 12.57 (br s, 1H), 8.83 (d, J=8.1 Hz, 1H), 8.23 (d, J=8.3 Hz, 1H), 8.01-7.92 (m, 1H), 7.84 (d, J=8.1 Hz, 1H), 7.68-7.45 (m, 6H), 7.37 (br s, 1H), 6.96 (d, J=8.3 Hz, 1H), 6.76 (d, J=2.4 Hz, 1H), 6.71 (dd, J=2.4, 8.2 Hz, 1H), 5.89 (quin, J=7.1 Hz, 1H), 2.14 (s, 3H), 1.55 (d, J=6.8 Hz, 3H).

### Example 16: (R)-2-Methyl-N-(1-(naphthalen-1-yl)ethyl)-5-(thiazol-2-ylamino)benzamide (Compound 107)

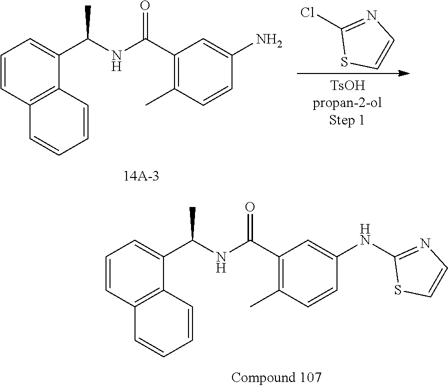 [SMILES: Cc1ccc(N)cc1C(=O)N[C@H](C)c1cccc2ccccc12 ; Clc1nccs1 ; Cc1ccc(Nc2nccs2)cc1C(=O)N[C@H](C)c1cccc2ccccc12]

### Step 1: (R)-2-Methyl-N-(1-(naphthalen-1-yl)ethyl)-5-(thiazol-2-ylamino)benzamide (Compound 107)

To a mixture of (R)-5-amino-2-methyl-N-(1-(naphthalen-1-yl)ethyl)benzamide (100 mg, 329 μmol, 1.5 eq) and 2-chlorothiazole (26.2 mg, 219 μmol, 1.0 eq) in propan-2-ol (2.0 mL) was added TOH·H₂O (62.5 mg, 329 μmol, 1.5 eq). The resulting mixture was stirred at 20° C. for 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 25%-55% B over 8 min; mobile phase A: water (10 mM NH₄HCO₃), mobile phase B: acetonitrile). (R)-2-Methyl-N-(1-(naphthalen-1-yl)ethyl)-5-(thiazol-2-ylamino)benzamide (16.8 mg, 43.4 μmol, 20% yield) was as an off-white solid. M+H⁺=388.2 (LCMS); ¹H NMR (400 MHZ, CDCl₃) δ 8.25 (d, J=8.3 Hz, 1H), 7.90 (d, J=8.2 Hz, 1H), 7.83 (d, J=8.2 Hz, 1H), 7.62-7.43 (m, 5H), 7.33-7.27 (m, 2H), 7.20 (d, J=3.5 Hz, 1H), 7.16 (d, J=8.2 Hz, 1H), 6.59 (d, J=3.7 Hz, 1H), 6.19-6.10 (m, 1H), 6.02 (br d, J=8.4 Hz, 1H), 2.40 (s, 3H), 1.82 (d, J=6.7 Hz, 3H).

### Example 17: (R)-5-((2-Methoxypyridin-3-yl)amino)-2-methyl-N-(1-(naphthalen-1-yl)ethyl)benzamide (Compound 115)

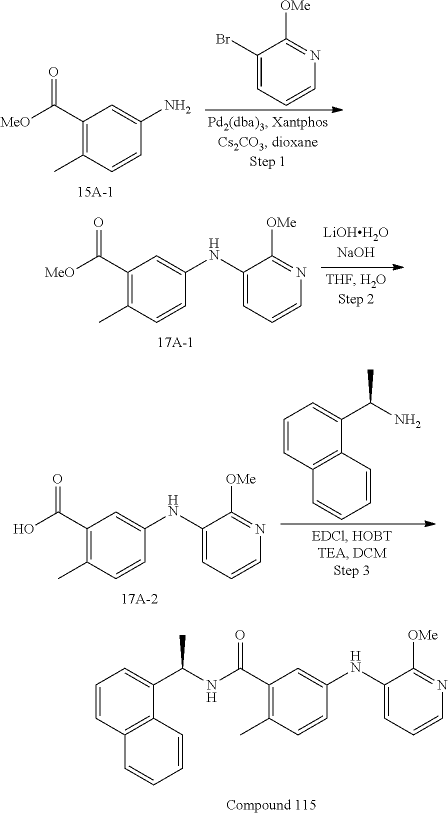 [SMILES: COC(=O)c1cc(N)ccc1C ; COc1ncccc1Br ; COC(=O)c1cc(Nc2cccnc2OC)ccc1C ; COc1ncccc1Nc1ccc(C)c(C(=O)O)c1 ; C[C@@H](N)c1cccc2ccccc12 ; COc1ncccc1Nc1ccc(C)c(C(=O)N[C@H](C)c2cccc3ccccc23)c1]

### Step 1: Methyl 5-((2-methoxypyridin-3-yl)amino)-2-methylbenzoate (17A-1)

A mixture of methyl 5-amino-2-methylbenzoate (400 mg, 2.42 mmol, 1.2 eq), 3-bromo-2-methoxypyridine (379 mg, 2.02 mmol, 1.0 eq) and Cs₂CO₃ (1.31 g, 4.04 mmol, 2.0 eq) in dioxane (6.0 mL) was degassed and purged with N₂ three times. To the mixture were added Pd₂ (dba) 3 (185 mg, 202 μmol, 0.1 eq) and Xantphos (234 mg, 404 μmol, 0.2 eq) at 20° C. The resulting mixture was stirred at 120° C. for 16 h under a N₂ atmosphere. LCMS indicated that the starting material was completely consumed, and the desired mass was detected. The reaction mixture was allowed to cool to room temperature, poured into H₂O (5.0 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/3. Methyl 5-((2-methoxypyridin-3-yl)amino)-2-methylbenzoate (400 mg, 1.47 mmol, 73% yield) was obtained as a yellow solid. M+H⁺=273.3 (LCMS).

### Step 2: 5-((2-Methoxypyridin-3-yl)amino)-2-methylbenzoic acid (17A-2)

To a solution of methyl 5-((2-methoxypyridin-3-yl)amino)-2-methylbenzoate (400 mg, 1.47 mmol, 1.0 eq) in a mixture of MeOH (9.0 mL) and THF (27 mL) was added LiOH·H₂O (185 mg, 4.41 mmol, 3.0 eq). The mixture was stirred at 70° C. for 5 h. TLC indicated that most of the starting material still remained. The mixture was allowed to cool to room temperature and NaOH (58.75 mg, 1.47 mmol, 1.0 eq) was added. The resulting mixture was stirred at 70° C. for another 16 h. LCMS indicated that the starting material was completely consumed, and the desired mass was detected. The reaction mixture was allowed to cool to room temperature, poured into H₂O (18 mL) and the mixture was washed with petroleum ether (6.0 mL×5). The aqueous layer was acidified to pH 5 with HCl (1 M aqueous). The product was extracted with EtOAc (6.0 mL×3). The combined organic layers were dried over Na₂SO₄, filtered, and concentrated under vacuum to give the crude product 5-((2-methoxypyridin-3-yl)amino)-2-methylbenzoic acid (210 mg) as a yellow oil, which was used in the next step without any further purification. M+H⁺=259.3 (LCMS).

### Step 3: (R)-5-((2-Methoxypyridin-3-yl)amino)-2-methyl-N-(1-(naphthalen-1-yl)ethyl)benzamide (Compound 115)

To a solution of (R)-1-(naphthalen-1-yl)ethanamine (66.3 mg, 387 μmol, 1.0 eq) and 5-((2-methoxypyridin-3-yl)amino)-2-methylbenzoic acid (100 mg, 387 μmol, 1.0 eq) in DCM (4.0 mL) were added TEA (206 mg, 2.03 mmol, 283 μL, 3.0 eq), EDCI (325 mg, 1.69 mmol, 2.5 eq) and HOBt (229 mg, 1.69 mmol, 2.5 eq). The mixture was stirred at 20° C. for 16 h. LCMS indicated that the starting material was completely consumed, and the desired mass was detected. The reaction mixture was poured into H₂O (4.0 mL) and extracted with DCM (2.0 mL×2). The combined organic layers were dried over Na₂SO₄, filtered, and concentrated under vacuum to give a residue which was purified by preparative TLC (EtOAc/petroleum ether=1/2, R_{f}=0.6). (R)-5-((2-Methoxypyridin-3-yl)amino)-2-methyl-N-(1-(naphthalen-1-yl)ethyl)benzamide (90.0 mg, 219 μmol, 57% yield) was obtained as a yellow solid. M+H⁺=412.2 (LCMS); ¹H NMR (400 MHZ, CDCl₃) δ 8.28-8.21 (m, 1H), 7.92-7.81 (m, 2H), 7.69-7.63 (m, 1H), 7.61-7.42 (m, 4H), 7.30-7.15 (m, 1H), 7.20-7.15 (m, 1H), 7.13-7.06 (m, 2H), 6.78-5.72 (m, 1H), 6.19-6.12 (m, 1H), 6.01-5.92 (m, 2H), 4.03 (s, 3H), 2.38 (s, 3H), 1.77-1.84 (m, 3H).

### Example 18: (R)-2-Methyl-N-(1-(naphthalen-1-yl)ethyl)-5-((2-oxo-1,2-dihydropyridin-3-yl)amino)benzamide (Compound 119)

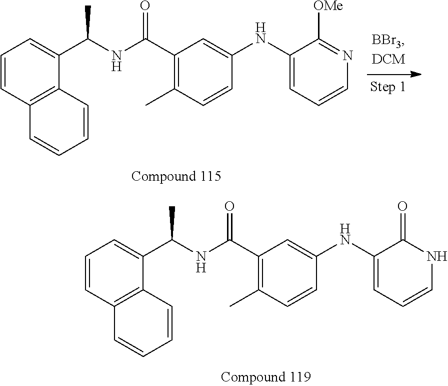 [SMILES: COc1ncccc1Nc1ccc(C)c(C(=O)N[C@H](C)c2cccc3ccccc23)c1 ; Cc1ccc(Nc2ccc[nH]c2=O)cc1C(=O)N[C@H](C)c1cccc2ccccc12]

### Step 1: (R)-2-Methyl-N-(1-(naphthalen-1-yl)ethyl)-5-((2-oxo-1,2-dihydropyridin-3-yl)amino)benzamide (Compound 119)

A mixture of (R)-5-((2-methoxypyridin-3-yl)amino)-2-methyl-N-(1-(naphthalen-1-yl)ethyl)benzamide (45 mg, 109 μmol, 1.0 eq) in DCM (5.0 mL) was degassed and purged with N₂ three times. To the mixture was added BBr₃ (219 mg, 875 μmol, 8.0 eq) in DCM (2 mL) at −78° C. The mixture was stirred at 20° C. for 2 h under a N₂ atmosphere. LCMS indicated that the starting material was completely consumed, and the desired mass was detected. The mixture was concentrated under vacuum to give a residue. The residue was diluted with MeOH (2 mL) and basified to pH 8 using NH₃·H₂O (37% aqueous). The mixture was concentrated under vacuum to give a residue which was purified by preparative HPLC (Phenomenex Gemini C18 column (80×30 mm, 3 μm); flow rate: 25 mL/min; gradient: 35%-65% B over 8 min; mobile phase A: 0.1% aqueous TFA, mobile phase B: acetonitrile). (R)-2-Methyl-N-(1-(naphthalen-1-yl)ethyl)-5-((2-oxo-1,2-dihydropyridin-3-yl)amino)benzamide (11.5 mg, 28.8 μmol, 26% yield, TFA salt) was obtained as a white solid. M+H⁺=398.0 (LCMS); ¹H NMR (400 MHZ, CD₃OD) δ 8.32-8.19 (m, 1H), 7.98-7.77 (m, 2H), 7.69-7.43 (m, 4H), 7.25-7.10 (m, 4H), 6.98-6.78 (m, 1H), 6.32-6.21 (m, 1H), 6.13-5.93 (m, 1H), 2.34-2.24 (m, 3H), 1.79-1.59 (m, 3H).

### Example 19: 5-(N-(1-Amino-3-hydroxypropan-2-yl) acetamido)-2-methyl-N—((R)-1-(naphthalen-1-yl)ethyl)benzamide (Compound 165)

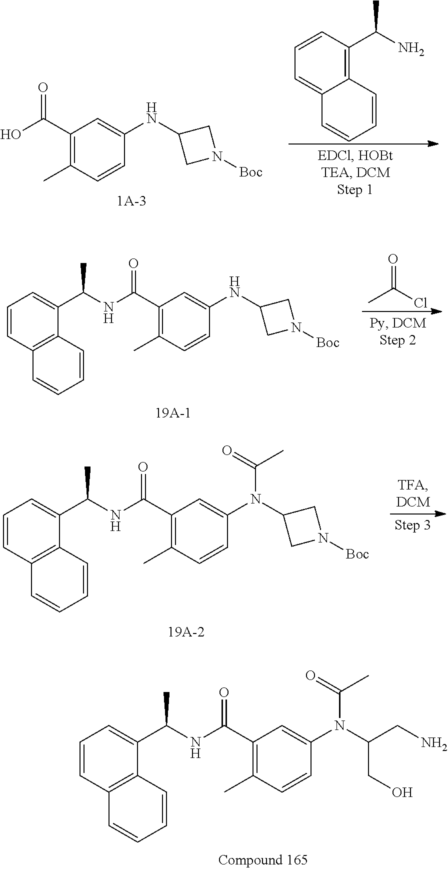

### Step 1: (R)-tert-butyl 3-((4-methyl-3-((1-(naphthalen-1-yl)ethyl)carbamoyl)phenyl)amino)azetidine-1-carboxylate (19A-1)

To a solution of 5-((1-(tert-butoxycarbonyl)azetidin-3-yl)amino)-2-methylbenzoic acid (100 mg, 326 μmol, 1.0 eq) and (R)-1-(naphthalen-1-yl)ethanamine (61.5 mg, 359 μmol, 1.1 eq) in DCM (5.0 mL) were added TEA (99.1 mg, 979 μmol, 136 μL, 3.0 eq), EDCI (75.1 mg, 392 μmol, 1.2 eq) and HOBt (52.9 mg, 392 μmol, 1.2 eq). The mixture was stirred at 20° C. for 3 h. TLC indicated that the starting material was completely consumed. The reaction mixture was poured into H₂O (5.0 mL) and extracted with EtOAc (2.0 mL×3). The combined organic layers were dried over Na₂SO₄, filtered, and concentrated under vacuum to give the crude product (R)-tert-butyl 3-((4-methyl-3-((1-(naphthalen-1-yl)ethyl)carbamoyl)phenyl)amino)azetidine-1-carboxylate (150 mg), which was used in the next step without any further purification. M+H⁺=460.4 (LCMS).

### Step 2: (R)-tert-Butyl 3-(N-(4-methyl-3-((1-(naphthalen-1-yl)ethyl)carbamoyl)phenyl)acetamido)azetidine-1-carboxylate (19A-2)

To a solution of (R)-tert-butyl 3-((4-methyl-3-((1-(naphthalen-1-yl)ethyl)carbamoyl)phenyl)amino)azetidine-1-carboxylate (40.0 mg, 87.0 μmol, 1.0 eq) and acetyl chloride (7.52 mg, 95.7 μmol, 6.83 μL, 1.1 eq) in DCM (1.0 mL) was added pyridine (6.88 mg, 87.0 μmol, 7.03 μL, 1.0 eq). The mixture was stirred at 20° C. for 3 h. LCMS indicated that the starting material was completely consumed, and the desired mass was detected. The reaction mixture was poured into H₂O (1.0 mL) and extracted with EtOAc (1.0 mL×3). The combined organic layers were dried over Na₂SO₄, filtered, and concentrated under vacuum to give the crude product (R)-tert-butyl 3-(N-(4-methyl-3-((1-(naphthalen-1-yl)ethyl)carbamoyl)phenyl) acetamido)azetidine-1-carboxylate (38.2 mg), which was used in the next step without any further purification. M+H⁺=502.4 (LCMS).

### Step 3: 5-(N-(1-Amino-3-hydroxypropan-2-yl) acetamido)-2-methyl-N—((R)-1-(naphthalen-1-yl)ethyl)benzamide (Compound 165)

To a solution of (R)-tert-butyl 3-(N-(4-methyl-3-((1-(naphthalen-1-yl)ethyl)carbamoyl)phenyl) acetamido)azetidine-1-carboxylate (10.0 mg, 19.9 μmol, 1.0 eq) in DCM (1.0 mL) was added TFA (154 mg, 1.35 mmol, 100 μL). The mixture was stirred at 20° C. for 30 min. LCMS indicated that the starting material was completely consumed, and the desired mass was detected. The reaction mixture was poured into H₂O (1.0 mL) and extracted with EtOAc (1.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 1%-30% B over 8 min; mobile phase A: 0.04% aqueous HCl, mobile phase B: acetonitrile). 5-(N-(1-Amino-3-hydroxypropan-2-yl) acetamido)-2-methyl-N—((R)-1-(naphthalen-1-yl)ethyl)benzamide (3.00 mg, 6.58 μmol, 33% yield, HCl salt) was obtained as a yellow gum. M+H⁺=420.1 (LCMS); ¹H NMR (400 MHz, CD₃OD) δ 8.25 (d, J=8.2 Hz, 1H), 7.90 (d, J=8.4 Hz, 1H), 7.81 (d, J=8.4 Hz, 1H), 7.64 (d, J=7.5 Hz, 1H), 7.60-7.42 (m, 3H), 7.03 (d, J=8.2 Hz, 1H), 6.79-6.66 (m, 2H), 6.05 (q, J=7.3 Hz, 1H), 4.20 (dd, J=4.0, 11.2 Hz, 1H), 4.04-3.89 (m, 2H), 3.23 (br dd, J=3.5, 13.1 Hz, 1H), 3.04-2.90 (m, 1H), 2.22 (s, 3H), 2.01 (d, J=4.4 Hz, 3H), 1.70 (dd, J=2.8, 6.9 Hz, 3H).

### Example 20: (R)-5-Methyl-N-(1-(naphthalen-1-yl)ethyl)-1H-indole-6-carboxamide (Compound 153)

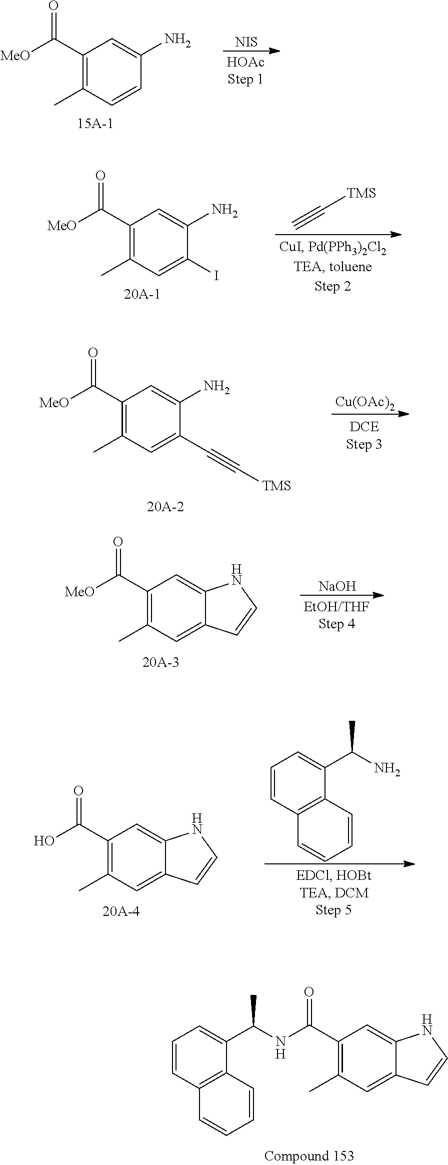

### Step 1: Methyl 5-amino-4-iodo-2-methylbenzoate (20A-1)

To a solution of methyl 5-amino-2-methylbenzoate (15.0 g, 90.8 mmol, 1.0 eq) in AcOH (80 mL) was added NIS (22.5 g, 99.9 mmol, 1.1 eq). The mixture was stirred at 20° C. for 1 h. TLC indicated that the starting material was consumed. The reaction mixture was poured into H₂O (200 mL) and extracted with EtOAc (100 mL×3). The combined organic layers were dried over Na₂SO₄, filtered, and concentrated under vacuum to give a residue which was purified by flash silica gel chromatography using a gradient of EtOAc/petroleum ether from 0/1 to 1/9. Methyl 5-amino-4-iodo-2-methylbenzoate (2.40 g, 9.18 mmol, 89% yield) was obtained as a brown oil. M+H⁺=293.0 (LCMS).

### Step 2: Methyl 5-amino-2-methyl-4-((trimethylsilyl)ethynyl)benzoate (20A-2)

A mixture of methyl 5-amino-4-iodo-2-methylbenzoate (3.00 g, 10.3 mmol, 1.0 eq), TEA (2.09 g, 20.6 mmol, 2.87 mL, 2.0 eq), Pd(PPh₃)₂Cl₂ (217 mg, 309 μmol, 0.03 eq), CuI (19.6 mg, 103 μmol, 0.01 eq) and ethynyltrimethylsilane (1.52 g, 15.5 mmol, 2.14 mL, 1.5 eq) in a mixture of toluene (80 mL) and H₂O (40 mL) was degassed and purged with N₂ for three times. The resulting mixture was stirred at 70° C. for 3 h under a N₂ atmosphere. TLC indicated that the starting material was completely consumed. The reaction mixture was allowed to cool to room temperature, poured into H₂O (30 mL) and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 0/1 to 1/4. Methyl 5-amino-2-methyl-4-((trimethylsilyl)ethynyl)benzoate (2.40 g, 9.18 mmol, 89% yield) was obtained as a brown oil. M+H⁺=262.1 (LCMS).

### Step 3: Methyl 5-methyl-1H-indole-6-carboxylate (20A-3)

A mixture of methyl 5-amino-2-methyl-4-((trimethylsilyl)ethynyl)benzoate (1.00 g, 3.83 mmol, 1.0 eq), Cu(OAc) 2 (1.39 g, 7.65 mmol, 2.0 eq) in DCE (50 mL) was degassed and purged with N₂ for three times, and then the mixture was stirred at 130° C. for 1 h under a N₂ atmosphere. TLC indicated that the starting material was completely consumed. The reaction mixture was allowed to cool to room temperature, poured into H₂O (100 mL) and extracted with DCM (50 mL×3). The combined organic layers were dried over Na₂SO₄, filtered, and concentrated under vacuum to give a residue which was purified by flash silica gel chromatography using a gradient of EtOAc/petroleum ether from 0/1 to 1/3. Methyl 5-methyl-1H-indole-6-carboxylate (500 mg, 2.64 mmol, 18% yield) was obtained as a yellow solid. M+H⁺=190.1 (LCMS).

### Step 4: 5-Methyl-1H-indole-6-carboxylic acid (20A-4)

To a solution of methyl 5-methyl-1H-indole-6-carboxylate (40.0 mg, 211 μmol, 1.0 eq) in a mixture of EtOH (2.0 mL) and THF (2.0 mL) was added NaOH (2 M aqueous, 529 μL, 5.0 eq). The mixture was stirred at 20° C. for 30 min. The mixture was stirred at 80° C. for 3 h. LCMS indicated that starting material was completely consumed, and the desired mass was detected. The reaction mixture was allowed to cool to room temperature, poured into H₂O (5.0 mL) and washed with MTBE (3.0 mL×3). The aqueous layer was acidified to pH 5 using HCl (1 M aqueous). The product was extracted with EtOAc (4.0 mL×3). The combined organic layers were dried over Na₂SO₄, filtered, and concentrated under vacuum to give 5-methyl-1H-indole-6-carboxylic acid (30.0 mg, 171 μmol, 81% yield) as a white solid, which was used in the next step without any further purification.

### Step 5: (R)-5-Methyl-N-(1-(naphthalen-1-yl)ethyl)-1H-indole-6-carboxamide (Compound 153)

To a solution of 5-methyl-1H-indole-6-carboxylic acid (30.0 mg, 171 μmol, 1.0 eq) and (R)-1-(naphthalen-1-yl)ethanamine (35.2 mg, 206 μmol, 33.0 μL, 1.2 eq) in DCM (2.0 mL) were added EDCI (39.4 mg, 206 μmol, 1.2 eq), HOBt (30.1 mg, 223 μmol, 1.3 eq) and TEA (52.0 mg, 514 μmol, 72.0 μL, 3.0 eq). The resulting mixture was stirred at 20° C. for 3 h. LCMS indicated that the starting material was completely consumed, and the desired mass was detected. The reaction mixture was poured into H₂O (5.0 mL) and extracted with DCM (2.0 mL×3). The combined organic layers were dried over Na₂SO₄, filtered, and concentrated under vacuum to give a residue which was purified by preparative TLC (EtOAc/petroleum ether=1:1, R_{f}=0.6). (R)-5-Methyl-N-(1-(naphthalen-1-yl)ethyl)-1H-indole-6-carboxamide (13.2 mg, 35.8 μmol, 21% yield) was obtained as a yellow solid. M+H⁺=329.0 (LCMS); ¹H NMR (400 MHz, CDCl₃) δ 8.29 (d, J=8.5 Hz, 1H), 8.18-8.09 (m, 1H), 7.90 (d, J=7.9 Hz, 1H), 7.83 (d, J=8.1 Hz, 1H), 7.62-7.45 (m, 4H), 7.43 (s, 1H), 7.38-7.33 (m, 1H), 7.22-7.17 (m, 1H), 6.49-6.39 (m, 1H), 6.22-6.13 (m, 1H), 6.02 (br d, J=8.4 Hz, 1H), 2.53 (s, 3H), 1.82 (d, J=6.8 Hz, 3H).

### Example 21: (R)-tert-Butyl((5-methyl-6-((1-(naphthalen-1-yl)ethyl)carbamoyl)-1H-indol-2-yl)methyl)carbamate (Compound 189)

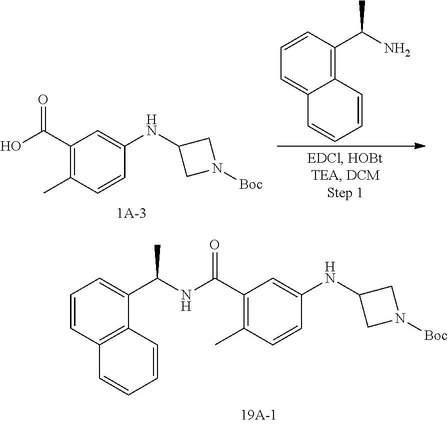 [SMILES: Cc1ccc(NC2CN(C(=O)OC(C)(C)C)C2)cc1C(=O)O ; C[C@@H](N)c1cccc2ccccc12 ; Cc1ccc(NC2CN(C(=O)OC(C)(C)C)C2)cc1C(=O)N[C@H](C)c1cccc2ccccc12]

### Step 1: Methyl 5-amino-4-(3-((tert-butoxycarbonyl)amino)prop-1-yn-1-yl)-2-methyl benzoate (21A-1)

To a mixture of methyl 5-amino-4-iodo-2-methylbenzoate (1.00 g, 3.44 mmol, 1.0 eq), tert-butyl prop-2-yn-1-ylcarbamate (533 mg, 3.44 mmol, 1.0 eq), CuI (262 mg, 1.37 mmol, 0.4 eq) and Pd(PPh₃)₂Cl₂ (482 mg, 687 μmol, 0.2 eq) was added TEA (10 mL) under a N₂ atmosphere. The mixture was stirred at 20° C. for 16 h. LCMS indicated that the starting material was completely consumed, and the desired mass was detected. The mixture was poured into H₂O (20 mL) and extracted with DCM (15 mL×8). The combined organic layers were dried over Na₂SO₄, filtered, and concentrated under vacuum to give a residue which was purified by flash silica gel chromatography using a gradient of EtOAc/petroleum ether from 0/1 to 1/1. Methyl 5-amino-4-(3-((tert-butoxycarbonyl)amino)prop-1-yn-1-yl)-2-methyl benzoate (1.50 g, 4.71 mmol, 51% yield) was obtained as a white solid. M+H⁺=319.2 (LCMS); ¹H NMR (400 MHZ, CDCl₃) δ 7.13-7.10 (m, 1H), 4.86-4.78 (m, 1H), 4.23-4.17 (m, 2H), 3.89-3.85 (m, 3H), 2.45-2.40 (m, 3H), 1.48 (s, 9H).

### Step 2: Methyl 2-(((tert-butoxycarbonyl)amino)methyl)-5-methyl-1H-indole-6-carboxylate (21A-2)

To a solution of methyl 5-amino-4-(3-((tert-butoxycarbonyl)amino)prop-1-yn-1-yl)-2-methyl benzoate (500 mg, 1.57 mmol, 1.0 eq) in DCE (30 mL) was added Cu(OAc) 2 (713 mg, 3.93 mmol, 2.5 eq). The mixture was stirred at 130° C. for 1 h under a N₂ atmosphere. TLC indicated that the starting material was completely consumed. The hot reaction mixture was poured into H₂O (20 mL) and extracted with EtOAc (20 mL×5). The combined organic layers were dried over Na₂SO₄, filtered, and concentrated under vacuum to give a residue which was purified by flash silica gel chromatography using a gradient of EtOAc/petroleum ether from 0/1 to 1/2. Methyl 2-(((tert-butoxycarbonyl)amino)methyl)-5-methyl-1H-indole-6-carboxylate (1.10 g, 3.46 mmol, 73% yield) was obtained as a yellow solid. ¹H NMR (400 MHZ, CDCl₃) δ 9.08-9.00 (m, 1H), 8.06-8.01 (m, 1H), 7.38 (s, 1H), 6.29-6.24 (m, 1H), 4.40-4.36 (m, 2H), 3.91-3.89 (m, 3H), 2.67 (s, 3H), 1.49 (s, 9H).

### Step 3: 2-(((tert-Butoxycarbonyl)amino)methyl)-5-methyl-1H-indole-6-carboxylic acid (21A-3)

To a solution of methyl 2-(((tert-butoxycarbonyl)amino)methyl)-5-methyl-1H-indole-6-carboxylate (800 mg, 2.51 mmol, 1.0 eq) in a mixture of THF (10 mL) and MeOH (3.0 mL) was added NaOH (2 M in aqueous, 8.0 mL, 6.4 eq). The mixture was stirred at 20° C. for 1 h and then was stirred at 60° C. for another 16 h. LCMS indicated that the starting material was completely consumed, and the desired mass was detected. The mixture was poured into H₂O (10 mL) and washed with MTBE (10 mL×2). The aqueous was acidified to pH 5 with HCl (1 M aqueous). The product was extracted with DCM (15 mL×5). The combined organic layers were dried over Na₂SO₄, filtered, and concentrated under vacuum to give 2-(((tert-butoxycarbonyl)amino)methyl)-5-methyl-1H-indole-6-carboxylic acid (750 mg, 1.97 mmol, 78% yield) as a red solid, which was used in the next step without any further purification. M−H⁻=303.1 (LCMS); ¹H NMR (400 MHZ, CDCl₃) δ 8.46-8.40 (m, 1H), 7.81-7.77 (m, 1H), 6.79-6.76 (m, 1H), 6.33-6.30 (m, 1H), 3.27-3.08 (m, 2H), 2.07-2.05 (m, 3H), 1.46-1.40 (m, 9H).

### Step 4: (R)-tert-Butyl((5-methyl-6-((1-(naphthalen-1-yl)ethyl)carbamoyl)-1H-indol-2-yl)methyl)carbamate (Compound 189)

To a solution of (R)-1-(naphthalen-1-yl)ethanamine (186 mg, 1.08 mmol, 174 μL, 1.1 eq) and 2-(((tert-butoxycarbonyl)amino)methyl)-5-methyl-1H-indole-6-carboxylic acid (300 mg, 986 μmol, 1.0 eq) in DCM (8.0 mL) were added TEA (299 mg, 2.96 mmol, 412 μL, 3.0 eq), EDCI (283 mg, 1.48 mmol, 1.5 eq) and HOBt (200 mg, 1.48 mmol, 1.5 eq). The mixture was stirred at 20° C. for 16 h. LCMS indicated that the starting material was completely consumed, and the desired mass was detected. The reaction mixture was poured into H₂O (10 mL) and extracted with DCM (10 mL×4). The combined organic layers were dried over Na₂SO₄, filtered, and concentrated under vacuum to give a residue which was purified by preparative HPLC (Phenomenex Gemini C18 column (150×40 mm, 10 μm); flow rate: 30 mL/min; gradient: 45%-75% B over 8 min; mobile phase A: 0.04% aqueous NH₄HCO₃, mobile phase B: acetonitrile). (R)-tert-Butyl((5-methyl-6-((1-(naphthalen-1-yl)ethyl)carbamoyl)-1H-indol-2-yl)methyl)carbamate (280 mg, 612 μmol, 62% yield) was obtained as a white solid. M+H⁺=458.1 (LCMS); ¹H NMR (400 MHz, CDCl₃) δ 8.89-8.82 (m, 1H), 8.32-8.26 (m, 1H), 7.92-7.87 (m, 1H), 7.85-7.81 (m, 1H), 7.62-7.45 (m, 4H), 7.34-7.28 (m, 2H), 6.22-6.12 (m, 2H), 6.04-5.98 (m, 1H), 5.07-5.00 (m, 1H), 4.35-4.30 (m, 2H), 2.53-2.50 (m, 3H), 1.84-1.79 (m, 3H), 1.48-1.44 (m, 9H).

### Example 22: (R)-2-(Aminomethyl)-5-methyl-N-(1-(naphthalen-1-yl)ethyl)-1H-indole-6-carboxamide (Compound 148)

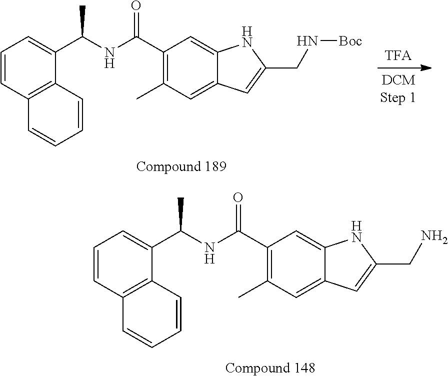 [SMILES: Cc1cc2cc(CNC(=O)OC(C)(C)C)[nH]c2cc1C(=O)N[C@H](C)c1cccc2ccccc12 ; Cc1cc2cc(CN)[nH]c2cc1C(=O)N[C@H](C)c1cccc2ccccc12]

### Step 1: (R)-2-(Aminomethyl)-5-methyl-N-(1-(naphthalen-1-yl)ethyl)-1H-indole-6-carboxamide (Compound 148)

To a stirred solution of (R)-tert-butyl((5-methyl-6-((1-(naphthalen-1-yl)ethyl)carbamoyl)-1H-indol-2-yl)methyl)carbamate (220 mg, 481 μmol, 1.0 eq) in DCM (8.0 mL) was added TFA (6.16 g, 54.0 mmol, 4.00 mL, 112 eq). The mixture was stirred at 20° C. for 1 h. LCMS indicated that the starting material was completely consumed, and the desired mass was detected. The mixture was concentrated under vacuum to give a residue which was purified by preparative HPLC (Phenomenex Gemini C18 column (75×30 mm, 3 μm); flow rate: 25 mL/min; gradient: 10%-40% B over 8 min; mobile phase A: 0.1% aqueous TFA, mobile phase B: acetonitrile). (R)-2-(Aminomethyl)-5-methyl-N-(1-(naphthalen-1-yl)ethyl)-1H-indole-6-carboxamide (150 mg, 420 μmol, 87% yield, TFA salt) was obtained as a pink powder. M+H⁺=358.1 (LCMS); ¹H NMR (400 MHZ, CD₃OD) δ 8.29 (d, J=8.6 Hz, 1H), 7.92 (d, J=7.9 Hz, 1H), 7.82 (d, J=8.3 Hz, 1H), 7.66 (d, J=7.1 Hz, 1H), 7.63-7.56 (m, 1H), 7.55-7.46 (m, 2H), 7.38 (d, J=5.4 Hz, 2H), 6.51 (s, 1H), 6.14-6.01 (m, 1H), 4.26 (s, 2H), 2.42 (s, 3H), 1.73 (d, J=6.9 Hz, 3H).

### Example 23: (R)-2-(Azetidin-3-yl)-5-methyl-N-(1-(naphthalen-1-yl)ethyl)-1H-indole-6-carboxamide (Compound 156)

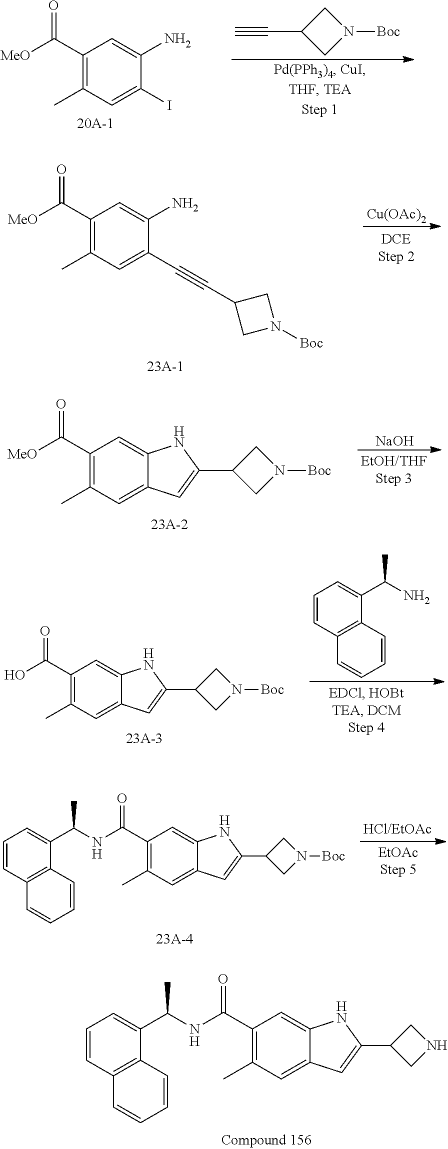

### Step 1: tert-Butyl 3-((2-amino-4-(methoxycarbonyl)-5-methylphenyl)ethynyl)azetidine-1-carboxylate (23A-1)

To a stirred solution of methyl 5-amino-4-iodo-2-methylbenzoate (300 mg, 1.03 mmol, 1.0 eq) in THF (6.0 mL) were added tert-butyl 3-ethynylazetidine-1-carboxylate (243 mg, 1.34 mmol, 1.3 eq), Pd(PPh₃)₄ (23.8 mg, 20.6 μmol, 0.02 eq), CuI (3.93 mg, 20.6 μmol, 0.02 eq) and TEA (261 mg, 2.58 mmol, 358.63 μL, 2.5 eq). Then the mixture was degassed and purged with N₂ three times and stirred at 20° C. for 16 h under a N₂ atmosphere. TLC indicated that the starting material was completely consumed. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative TLC (EtOAc/petroleum ether=1:1, R_{f}=0.7). tert-Butyl 3-((2-amino-4-(methoxycarbonyl)-5-methylphenyl)ethynyl)azetidine-1-carboxylate (300 mg, 871 μmol, 85% yield) was obtained as a yellow oil. ¹H NMR (400 MHZ, CDCl₃) δ 7.29 (s, 1H), 7.14 (s, 1H), 4.17-3.99 (m, 5H), 3.87 (s, 3H), 2.44 (s, 3H), 1.49-1.41 (m, 9H).

### Step 2: Methyl 2-(1-(tert-butoxycarbonyl)azetidin-3-yl)-5-methyl-1H-indole-6-carboxylate (23A-2)

To a solution of tert-butyl 3-((2-amino-4-(methoxycarbonyl)-5-methylphenyl)ethynyl) azetidine-1-carboxylate (160 mg, 465 μmol, 1.0 eq) in DCE (10 mL) was added Cu(OAc) 2 (169 mg, 929 μmol, 2.0 eq). The mixture was stirred at 130° C. for 2 h. TLC indicated that the starting material was completely consumed. The reaction mixture was allowed to cool to room temperature, poured into H₂O (10 mL) and extracted with EtOAc (4.0 mL×3). The combined organic layers were dried over Na₂SO₄, filtered, and concentrated under vacuum to give the crude product methyl 2-(1-(tert-butoxycarbonyl)azetidin-3-yl)-5-methyl-1H-indole-6-carboxylate (130 mg) as a brown oil, which was used in the next step without any further purification.

### Step 3: 2-(1-(tert-Butoxycarbonyl)azetidin-3-yl)-5-methyl-1H-indole-6-carboxylic acid (23A-3)

To a solution of methyl 2-(1-(tert-butoxycarbonyl)azetidin-3-yl)-5-methyl-1H-indole-6-carboxylate (80.0 mg, 42.1 μmol, 1.0 eq) in a mixture of EtOH (2.0 mL) and THF (2.0 mL) was added NaOH (2 M aqueous, 2.0 mL, 24 eq). The mixture was stirred at 80° C. for 16 h. LCMS indicated that starting material was completely consumed, and the desired mass was detected. The reaction mixture was allowed to cool to room temperature, poured into H₂O (5.0 mL) and washed with MTBE (3.0 mL×2). The aqueous layer was acidified to pH 5 with HCl (1 M aqueous). The product was extracted with EtOAc (2.0 mL×3). The combined organic layers were dried over Na₂SO₄, filtered, and concentrated under vacuum to give the crude product 2-(1-(tert-butoxycarbonyl)azetidin-3-yl)-5-methyl-1H-indole-6-carboxylic acid (80.0 mg) as a white oil, which was used in the next step without any further purification. M−56+H⁺=275.2 (LCMS).

### Step 4: (R)-tert-Butyl 3-(5-methyl-6-((1-(naphthalen-1-yl)ethyl)carbamoyl)-1H-indol-2-yl)azetidine-1-carboxylate (23A-4)

To a solution of 2-(1-(tert-butoxycarbonyl)azetidin-3-yl)-5-methyl-1H-indole-6-carboxylic acid (100 mg, 303 μmol, 1.0 eq) and (R)-1-(naphthalen-1-yl)ethanamine (57.0 mg, 333 μmol, 53.3 μL, 1.1 eq) in DCM (4.0 mL) were added EDCI (69.6 mg, 363 μmol, 1.2 eq), HOBt (49.1 mg, 363 μmol, 1.2 eq) and TEA (91.9 mg, 909 μmol, 126 μL, 3.0 eq). The resulting mixture was stirred at 20° C. for 3 h. LCMS indicated that the starting material was completely consumed, and the desired mass was detected. The reaction mixture was poured into H₂O (5.0 mL) and extracted with DCM (2.0 mL×3). The combined organic layers were dried over Na₂SO₄, filtered, and concentrated under vacuum to give a residue which was purified by preparative TLC (EtOAc/petroleum ether=1:1, R_{f}=0.6). (R)-tert-Butyl 3-(5-methyl-6-((1-(naphthalen-1-yl)ethyl)carbamoyl)-1H-indol-2-yl)azetidine-1-carboxylate (50.0 mg, 103 μmol, 34% yield) was obtained as a white solid. M+H⁺=484.1 (LCMS).

### Step 5: (R)-2-(Azetidin-3-yl)-5-methyl-N-(1-(naphthalen-1-yl)ethyl)-1H-indole-6-carboxamide (Compound 156)

To a solution of (R)-tert-butyl 3-(5-methyl-6-((1-(naphthalen-1-yl)ethyl)carbamoyl)-1H-indol-2-yl)azetidine-1-carboxylate (50.0 mg, 103 μmol, 1.0 eq) in EtOAc (3.0 mL) was added HCl/EtOAc (4 M, 1.0 mL). The resulting mixture was stirred at 20° C. for 1 h. LCMS indicated that the starting material was completely consumed, and the desired mass was detected. The reaction mixture was concentrated under vacuum to give a residue which was purified by preparative HPLC (Phenomenex Luna C18 column (80×30 mm, 3 μm); flow rate: 25 mL/min; gradient: 15%-30% B over 8 min; mobile phase A: 0.04% aqueous HCl, mobile phase B: acetonitrile). (R)-2-(Azetidin-3-yl)-5-methyl-N-(1-(naphthalen-1-yl)ethyl)-1H-indole-6-carboxamide (5.95 mg, 14.2 μmol, 14% yield, HCl salt) was obtained as a white gum. M+H=384.1 (LCMS); ¹H NMR (400 MHZ, CD₃OD) δ 8.28 (d, J=8.3 Hz, 1H), 7.91 (d, J=8.1 Hz, 1H), 7.82 (d, J=8.1 Hz, 1H), 7.66 (d, J=7.0 Hz, 1H), 7.62-7.43 (m, 3H), 7.34 (d, J=13.0 Hz, 2H), 6.46 (s, 1H), 6.07 (q, J=6.7 Hz, 1H), 4.48-4.23 (m, 5H), 2.42 (s, 3H), 1.73 (d, J=7.0 Hz, 3H).

### Example 24: (R)-2-((Dimethylamino)methyl)-1-(hydroxymethyl)-5-methyl-N-(1-(naphthalen-1-yl)ethyl)-1H-indole-6-carboxamide (Compound 192)

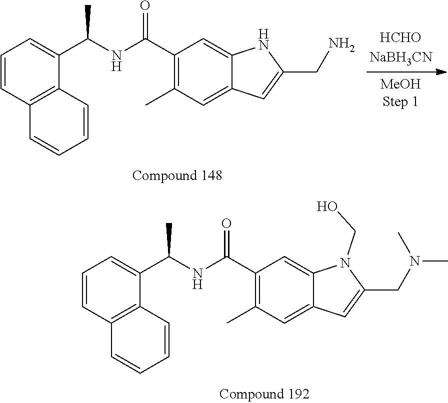 [SMILES: Cc1cc2cc(CN)[nH]c2cc1C(=O)N[C@H](C)c1cccc2ccccc12 ; Cc1cc2cc(CN(C)C)n(CO)c2cc1C(=O)N[C@H](C)c1cccc2ccccc12]

### Step 1: (R)-2-((Dimethylamino)methyl)-1-(hydroxymethyl)-5-methyl-N-(1-(naphthalen-1-yl)ethyl)-1H-indole-6-carboxamide (Compound 192)

To a solution of (R)-2-(aminomethyl)-5-methyl-N-(1-(naphthalen-1-yl)ethyl)-1H-indole-6-carboxamide (90.0 mg, 252 μmol, 1.0 eq) in MeOH (6.0 mL) was added TEA (50.0 μL), followed by the addition of formaldehyde (2.94 g, 36.3 mmol, 2.70 mL, 37% purity in water, 144 eq). The resulting mixture was adjusted to pH 6 with a small amount of AcOH. The mixture was stirred at 20° C. for 30 min, then NaBH₃CN (134 mg, 2.13 mmol, 2.0 eq) was added. The resulting reaction mixture was stirred at 20° C. for another 16 h. LCMS indicated that the starting material was completely consumed, and the desired mass was detected. The mixture was treated with H₂O (10 mL) and extracted with DCM (10 mL×5). The combined organic layers were dried over Na₂SO₄, filtered, and concentrated under vacuum to give a residue which was purified by preparative HPLC (Phenomenex Gemini C18 column (200×40 mm, 10 μm); flow rate: 50 mL/min; gradient: 5%-40% B over 8 min; mobile phase A: 0.2% aqueous FA, mobile phase B: acetonitrile). (R)-2-((Dimethylamino)methyl)-1-(hydroxymethyl)-5-methyl-N-(1-(naphthalen-1-yl)ethyl)-1H-indole-6-carboxamide (22.3 mg, 48.1 μmol, 19% yield, FA salt) was obtained as a white solid. M+H⁺=416.1 (LCMS); ¹H NMR (400 MHZ, DMSO-d₆) δ 8.78-8.73 (m, 1H), 8.30-8.25 (m, 1H), 8.21-8.18 (m, 1H), 7.98-7.94 (m, 1H), 7.86-7.81 (m, 1H), 7.68-7.65 (m, 1H), 7.63-7.49 (m, 4H), 7.32-7.28 (m, 1H), 6.35-6.33 (m, 1H), 5.98-5.90 (m, 1H), 5.62-5.54 (m, 2H), 3.65-3.60 (m, 2H), 2.35-2.33 (m, 3H), 2.20-2.14 (m, 6H), 1.63-1.57 (m, 3H).

### Example 25: (R)-5-Methyl-2-(morpholinomethyl)-N-(1-(naphthalen-1-yl)ethyl)-1H-indole-6-carboxamide (Compound 206)

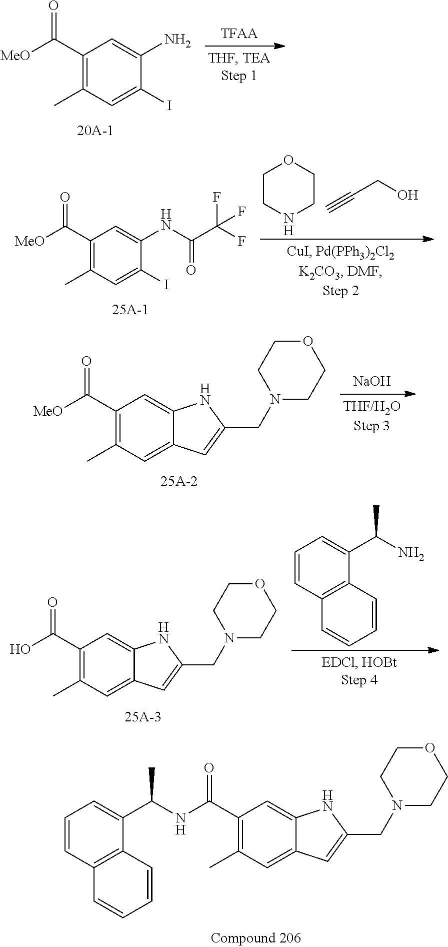

### Step 1: Methyl 4-iodo-2-methyl-5-(2,2,2-trifluoroacetamido)benzoate (25A-1)

To a stirred solution of methyl 5-amino-4-iodo-2-methylbenzoate (300 mg, 1.03 mmol, 1.0 eq) and TEA (125 mg, 1.24 mmol, 172 μL, 1.2 eq) in THF (3.5 mL) was added dropwise a solution of TFAA (238 mg, 1.13 mmol, 158 μL, 1.1 eq) in THF (1.0 mL) at −15° C. The mixture was stirred at the same temperature for 1 h then at 25° C. for 16 h. LCMS indicated that the starting material was completely consumed, and the desired compound was detected. The mixture was poured into water (10 mL) and extracted with EtOAc (5.0 mL×2). The combined organic layers were dried over Na₂SO₄, filtered, and concentrated under vacuum to give crude methyl 4-iodo-2-methyl-5-(2,2,2-trifluoroacetamido)benzoate (400 mg) as a yellow solid which was used the next step without any further purification. M−H⁻=386.0 (LCMS).

### Step 2: Methyl 5-methyl-2-(morpholinomethyl)-1H-indole-6-carboxylate (25A-2)

To a mixture of CuI (4.92 mg, 25.8 μmol, 0.04 eq) and Pd(PPh₃)₂Cl₂ (2.27 mg, 3.23 μmol, 0.005 eq) in DMF (4.5 mL) were added methyl 4-iodo-2-methyl-5-(2,2,2-trifluoroacetamido)benzoate (250 mg, 646 μmol, 1.0 eq), morpholine (113 mg, 1.29 mmol, 114 μL, 2.0 eq), prop-2-yn-1-ol (54.3 mg, 969 μmol, 57.2 μL, 1.5 eq) and K₂CO₃ (179 mg, 1.29 mmol, 2.0 eq) followed by DMF (2.0 mL) under a N₂ atmosphere. The mixture was stirred at 80° C. for 2 h. TLC indicated that the starting material was completely consumed. The reaction mixture was cooled to room temperature and diluted with MTBE (10 mL). The resulting mixture was washed with saturated aqueous NaHCO₃ (5.0 mL×2). The organic layer was dried over Na₂SO₄, filtered, and concentrated under vacuum to give a residue which was purified by flash silica gel chromatography using a gradient of EtOAc/petroleum ether from 1/100 to 1/1. Methyl 5-methyl-2-(morpholinomethyl)-1H-indole-6-carboxylate (170 mg, 85% yield) was obtained as a yellow oil.

### Step 3: 5-Methyl-2-(morpholinomethyl)-1H-indole-6-carboxylic acid (25A-3)

To a solution of methyl 5-methyl-2-(morpholinomethyl)-1H-indole-6-carboxylate (170 mg, 590 μmol, 1.0 eq) in a mixture of THF (10 mL) and H₂O (2.5 mL) was added NaOH (2 M aqueous, 7.37 mL, 25 eq). The mixture was stirred at 20° C. for 1 h then at 70° C. for 16 h. LCMS indicated that half of the starting material remained and another NaOH (2 M aqueous, 5.0 mL) aqueous was added, and the mixture was stirred another 5 h. The reaction mixture was allowed to cool to room temperature and washed with MTBE (10 mL). The aqueous was acidified to pH 5 with HCl (1 M aqueous) and extracted with DCM (5.0 mL×5). The organic layers were dried over Na₂SO₄, filtered, and concentrated under vacuum to give 5-methyl-2-(morpholinomethyl)-1H-indole-6-carboxylic acid (175 mg, 90% purity) as a brown gum.

### Step 4: (R)-5-Methyl-2-(morpholinomethyl)-N-(1-(naphthalen-1-yl)ethyl)-1H-indole-6-carboxamide (Compound 206)

To a mixture of 5-methyl-2-(morpholinomethyl)-1H-indole-6-carboxylic acid (140 mg, 510 μmol, 1.0 eq) and (R)-1-(naphthalen-1-yl)ethanamine (87.4 mg, 510 μmol, 81.7 μL, 1.0 eq) in DCM (2.0 mL) were added EDCI (147 mg, 766 μmol, 1.5 eq) and HOBt (103 mg, 766 μmol, 1.5 eq), followed by TEA (155 mg, 1.53 mmol, 213 μL, 3.0 eq). The mixture was stirred at 20° C. for 16 h. LCMS indicated that the starting material was completely consumed, and the desired mass was detected. The reaction mixture was poured into H₂O (5.0 mL) and extracted with DCM (5.0 mL×2). The combined organic layers were dried over Na₂SO₄, filtered, and concentrated under vacuum to give a residue which was purified by preparative HPLC (Phenomenex Gemini C18 column (75×30 mm, 3 μm); flow rate: 25 mL/min; gradient: 20%-60% B over 8 min; mobile phase A: 0.2% aqueous FA, mobile phase B: acetonitrile). (R)-5-Methyl-2-(morpholinomethyl)-N-(1-(naphthalen-1-yl)ethyl)-1H-indole-6-carboxamide (18.4 mg, 43.0 μmol, 8% yield, FA salt) was obtained as a white solid. M+H⁺=428.2 (LCMS); 1H NMR (400 MHZ, CDCl₃) δ 9.43-9.32 (m, 1H), 8.41-8.37 (m, 1H), 8.32-8.25 (m, 1H), 7.93-7.87 (m, 1H), 7.86-7.79 (m, 1H), 7.63-7.44 (m, 4H), 7.37-7.31 (m, 2H), 6.35-6.27 (m, 1H), 6.22-6.12 (m, 1H), 6.07-5.96 (m, 1H), 3.84-3.69 (m, 6H), 2.63-2.49 (m, 7H), 1.82 (d, J=6.8 Hz, 3H).

### Example 26: (R)—N-(1-(Naphthalen-1-yl)ethyl)-1H-benzo[d]imidazole-6-carboxamide (Compound 125)

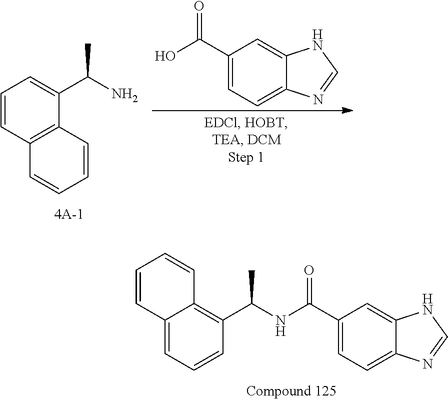 [SMILES: C[C@@H](N)c1cccc2ccccc12 ; O=C(O)c1ccc2nc[nH]c2c1 ; C[C@@H](NC(=O)c1ccc2nc[nH]c2c1)c1cccc2ccccc12]

### Step 1: (R)—N-(1-(Naphthalen-1-yl)ethyl)-1H-benzo[d]imidazole-6-carboxamide (Compound 125)

To a solution of (R)-1-(naphthalen-1-yl)ethanamine (100 mg, 584 μmol, 1.0 eq) and 1H-benzo[d]imidazole-6-carboxylic acid (94.7 mg, 584 μmol, 1.0 eq) in DCM (4.0 mL) were added TEA (118 mg, 1.17 mmol, 163 μL, 2.0 eq), EDCI (134 mg, 701 μmol, 1.2 eq) and HOBT (94.7 mg, 701 μmol, 1.2 eq). The mixture was stirred at 20° C. for 3 h. LCMS indicated that the starting material was completely consumed, and the desired mass was detected. The reaction mixture was poured into H₂O (4.0 mL) and extracted with DCM (4.0 mL×2). The combined organic layers were dried over Na₂SO₄, filtered, and concentrated under vacuum to give a residue which was purified by preparative HPLC (Phenomenex Gemini C18 column (100×30 mm, 10 μm); flow rate: 25 mL/min; gradient: 20%-50% B over 8 min; mobile phase A: 10 mM aqueous NH₄HCO₃, mobile phase B: acetonitrile). (R)—N-(1-(Naphthalen-1-yl)ethyl)-1H-benzo[d]imidazole-6-carboxamide (40.7 mg, 121 μmol, 21% yield) was obtained as a white solid. M+H⁺=316.3 (LCMS); ¹H NMR (400 MHZ, CDCl₃) δ 8.11-8.07 (m, 2H), 7.84-7.78 (m, 1H), 7.76 (s, 1H), 7.71-7.66 (m, 1H), 7.56-7.48 (m, 2H), 7.44-7.31 (m, 4H), 7.14-7.02 (m, 1H), 6.11-6.02 (m, 1H), 1.77-1.65 (m, 3H).

### Example 27: (R)-5-Methyl-N-(1-(naphthalen-1-yl)ethyl)-1H-benzo[d]imidazole-6-carboxamide (Compound 102)

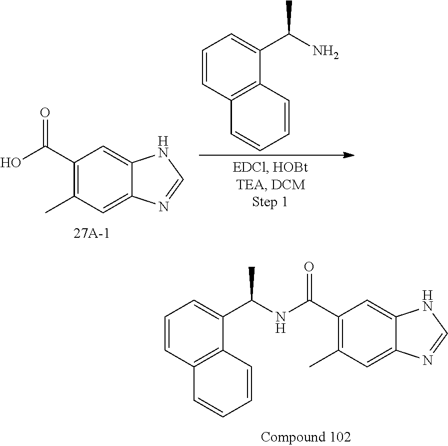 [SMILES: Cc1cc2nc[nH]c2cc1C(=O)O ; C[C@@H](N)c1cccc2ccccc12 ; Cc1cc2nc[nH]c2cc1C(=O)N[C@H](C)c1cccc2ccccc12]

### Step 1: (R)-5-Methyl-N-(1-(naphthalen-1-yl)ethyl)-1H-benzo[d]imidazole-6-carboxamide (Compound 102)

To a mixture of (R)-1-(naphthalen-1-yl)ethanamine (40.3 mg, 235 μmol, 37.6 μL, 1.0 eq) and 5-methyl-1H-benzo[d]imidazole-6-carboxylic acid (50.0 mg, 235 μmol, 1.0 eq, HCl salt) in DCM (3.0 mL) were added EDCI (67.6 mg, 353 μmol, 1.5 eq), HOBt (47.7 mg, 353 μmol, 1.5 eq) and TEA (71.4 mg, 705 μmol, 98.2 μL, 3.0 eq). The resulting mixture was stirred at 20° C. for 3 h. LCMS indicated that the starting material was completely consumed, and the desired mass was detected. The reaction mixture was poured into H₂O (5.0 mL) and extracted with DCM (2.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 25%-55% B over 8 min; mobile phase A: 10 mM aqueous NH₄HCO₃, mobile phase B: acetonitrile). (R)-5-Methyl-N-(1-(naphthalen-1-yl)ethyl)-1H-benzo[d]imidazole-6-carboxamide (16.1 mg, 48.0 μmol, 20% yield) as a white solid. M+H⁺=330.2 (LCMS); ¹H NMR (400 MHZ, DMSO-d₆) δ 12.42 (s, 1H), 8.95-8.78 (m, 1H), 8.26 (d, J=8.4 Hz, 1H), 8.23-8.17 (m, 1H), 7.96 (d, J=7.9 Hz, 1H), 7.84 (br d, J=7.9 Hz, 1H), 7.69-7.49 (m, 4H), 7.47 (s, 1H), 7.34 (s, 1H), 6.00-5.86 (m, 1H), 2.44-2.36 (m, 3H), 1.60 (d, J=7.0 Hz, 3H).

### Example 28: (R)-2-(Aminomethyl)-5-methyl-N-(1-(naphthalen-1-yl)ethyl)-1H-benzo[d]imidazole-6-carboxamide (Compound 128)

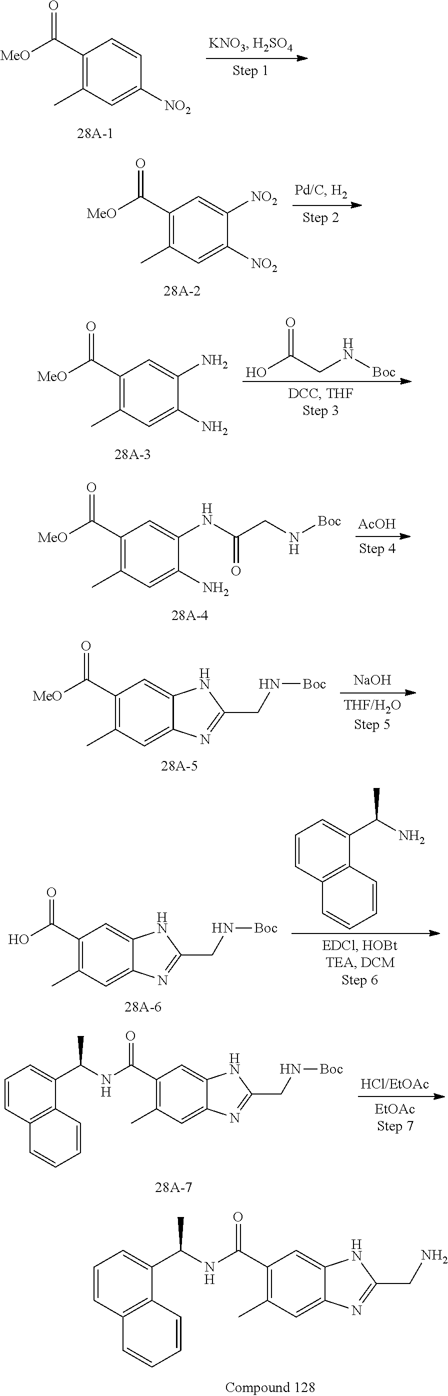

### Step 1: Methyl 2-methyl-4,5-dinitrobenzoate (28A-2)

To a solution of methyl 2-methyl-4-nitrobenzoate (1.00 g, 5.12 mmol, 1.0 eq) in H₂SO₄ (10 mL, 98% purity) was added KNO₃ (596 mg, 5.89 mmol, 1.2 eq) at 0° C. The resulting mixture was stirred at 20° C. for 16 h. TLC indicated that the starting material was completely consumed. The reaction mixture was poured into ice water (20 mL) and neutralized with saturated aqueous NaHCO₃ to adjust the pH to 7. The product was extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/100 to 1/5. Methyl 2-methyl-4,5-dinitrobenzoate (700 mg, 2.91 mmol, 57% yield) was obtained as a yellow solid. ¹H NMR (400 MHZ, DMSO-d₆) δ 8.54 (s, 1H), 8.26 (s, 1H), 3.98-3.84 (m, 3H), 2.73-2.58 (m, 3H).

### Step 2: Methyl 4,5-diamino-2-methylbenzoate (28A-3)

To a solution of methyl 2-methyl-4,5-dinitrobenzoate (1.50 g, 6.25 mmol, 1.0 eq) and 10% palladium on carbon (100 mg) in a mixture of MeOH (20 mL) and dioxane (20 mL) was degassed and purged with H₂ for three times. The mixture was stirred at 20° C. for 16 h under H₂ (15 psi) atmosphere. LCMS indicated that the starting material was completely consumed, and the desired mass was detected. The suspension was filtered through a pad of Celite, and the filter cake was washed with EtOAc (2.0 mL×3). The combined filtrates were concentrated under vacuum to give methyl 4,5-diamino-2-methylbenzoate (600 mg, 3.33 mmol, 53% yield) as a brown solid. M+H⁺=181.2 (LCMS).

### Step 3: Methyl 4-amino-5-(2-((tert-butoxycarbonyl)amino) acetamido)-2-methylbenzoate (28A-4)

To a solution of methyl 4,5-diamino-2-methylbenzoate (170 mg, 943 μmol, 1.0 eq) and 2-((tert-butoxycarbonyl)amino) acetic acid (165 mg, 943 μmol, 1.0 eq) in THF (5.0 mL) was added DCC (389 mg, 1.89 mmol, 382 μL, 2.0 eq). The resulting mixture was stirred at 20° C. for 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by flash silica gel chromatography using a gradient of EtOAc/petroleum ether from 1/50 to 1/5. Methyl 4-amino-5-(2-((tert-butoxycarbonyl)amino) acetamido)-2-methylbenzoate (200 mg, 593 μmol, 63% yield) was obtained as a white solid. M+H⁺=338.3 (LCMS); ¹H NMR (400 MHZ, CDCl₃) δ 8.09 (br s, 1H), 7.85-7.72 (m, 1H), 6.67-6.56 (m, 1H), 5.43 (br s, 1H), 4.01-3.91 (m, 2H), 3.84-3.73 (m, 3H), 2.56-2.37 (m, 3H), 1.54-1.43 (m, 9H).

### Step 4: Methyl 2-(((tert-butoxycarbonyl)amino)methyl)-5-methyl-1H-benzo[d]imidazole-6-carboxylate (28A-5)

To a solution of methyl 4-amino-5-(2-((tert-butoxycarbonyl)amino) acetamido)-2-methylbenzoate (100 mg, 296 μmol, 1.0 eq) in AcOH (1.0 mL). The resulting mixture was stirred at 80° C. for 2 h. LCMS indicated that the starting material was completely consumed, and the desired mass was detected. The reaction mixture was allowed to cool to room temperature, poured into H₂O (5.0 mL) and extracted with EtOAc (2.0 mL×3). The combined organic layers were dried over Na₂SO₄, filtered, and concentrated under vacuum to give crude product methyl 2-(((tert-butoxycarbonyl)amino)methyl)-5-methyl-1H-benzo[d]imidazole-6-carboxylate (90.0 mg) as a colorless oil, which was used in the next step without any further purification. M+H⁺=320.3 (LCMS).

### Step 5: 2-(((tert-Butoxycarbonyl)amino)methyl)-5-methyl-1H-benzo[d]imidazole-6-carboxylic acid (28A-6)

To a solution of methyl 2-(((tert-butoxycarbonyl)amino)methyl)-5-methyl-1H-benzo[d]imidazole-6-carboxylate (90.0 mg, 282 μmol, 1.0 eq) in a mixture of THF (9.0 mL) and H₂O (3.0 mL) was added NaOH (2 M aqueous, 423 μL, 3.0 eq). The mixture was stirred at 80° C. for 16 h. LCMS indicated that starting material was completely consumed, and the desired mass was detected. The reaction mixture was allowed to cool to room temperature, poured into H₂O (10.0 mL) and washed with MTBE (5.0 mL×2). The aqueous layer was acidified to pH 5 using HCl (1 M aqueous). The product was extracted with EtOAc (4.0 mL×3). The combined organic layers were dried over Na₂SO₄, filtered, and concentrated under vacuum to give the crude product 2-(((tert-butoxycarbonyl)amino)methyl)-5-methyl-1H-benzo[d]imidazole-6-carboxylic acid (90.0 mg) as a colorless oil, which was used in the next step without any further purification. M+H⁺=306.3 (LCMS).

### Step 6: (R)-tert-Butyl((5-methyl-6-((1-(naphthalen-1-yl)ethyl)carbamoyl)-1H-benzo[d]imidazol-2-yl)methyl)carbamate (28A-7)

To a solution of 1-(naphthalen-1-yl)cyclopropanamine (33.7 mg, 197 μmol, 31.5 μL, 1.0 eq) and 2-(((tert-butoxycarbonyl)amino)methyl)-5-methyl-1H-benzo[d]imidazole-6-carboxylic acid (60.0 mg, 197 μmol, 1.0 eq) in DCM (2.0 mL) were added TEA (59.7 mg, 590 μmol, 82.1 μL, 3.0 eq), EDCI (45.2 mg, 236 μmol, 1.2 eq) and HOBt (31.9 mg, 236 μmol, 1.2 eq). The mixture was stirred at 20° C. for 3 h. LCMS indicated that starting material was completely consumed, and the desired mass was detected. The reaction mixture was poured into H₂O (5.0 mL) and extracted with DCM (3.0 mL×3). The combined organic layers were dried over Na₂SO₄, filtered, and concentrated under vacuum to give a residue which was purified by preparative TLC (EtOAc/petroleum ether=1:1, R_{f}=0.5). (R)-tert-Butyl((5-methyl-6-((1-(naphthalen-1-yl)ethyl)carbamoyl)-1H-benzo[d]imidazol-2-yl)methyl)carbamate (40.0 mg, 87.0 μmol, 44% yield) was obtained as a white solid. M+H⁺=459.4 (LCMS).

### Step 7: (R)-2-(Aminomethyl)-5-methyl-N-(1-(naphthalen-1-yl)ethyl)-1H-benzo[d]imidazole-6-carboxamide (Compound 128)

To a stirred solution of (R)-tert-butyl((5-methyl-6-((1-(naphthalen-1-yl)ethyl)carbamoyl)-1H-benzo[d]imidazol-2-yl)methyl)carbamate (40.0 mg, 87.0 μmol, 1.0 eq) in EtOAc (2.0 mL) was added HCl/EtOAc (4 M, 1.0 mL). The mixture was stirred at 20° C. for 1 h. LCMS indicated that starting material was completely consumed, and the desired mass was detected. The mixture was concentrated under vacuum to give a residue which was purified by preparative HPLC (Phenomenex Luna C18 column (150×30 mm, 5 μm); flow rate: 25 mL/min; gradient: 5%-40% B over 8 min; mobile phase A: 0.1% aqueous TFA, mobile phase B: acetonitrile). (R)-2-(Aminomethyl)-5-methyl-N-(1-(naphthalen-1-yl)ethyl)-1H-benzo[d]imidazole-6-carboxamide (9.70 mg, 20.5 μmol, 24% yield, TFA salt) was obtained as a white solid. M+H⁺=359.0 (LCMS); ¹H NMR (400 MHZ, CD₃OD) δ 8.32 (d, J=8.7 Hz, 1H), 7.95 (d, J=8.2 Hz, 1H), 7.86 (d, J=8.3 Hz, 1H), 7.69 (d, J=7.3 Hz, 1H), 7.65-7.50 (m, 4H), 7.47 (s, 1H), 6.12 (q, J=6.7 Hz, 1H), 4.42 (s, 2H), 2.50 (s, 3H), 1.77 (d, J=7.0 Hz, 3H).

### Example 29: (R)-5-Methyl-N-(1-(naphthalen-1-yl)ethyl)-1H-benzo[d]imidazole-6-carboxamide (Compound 131)

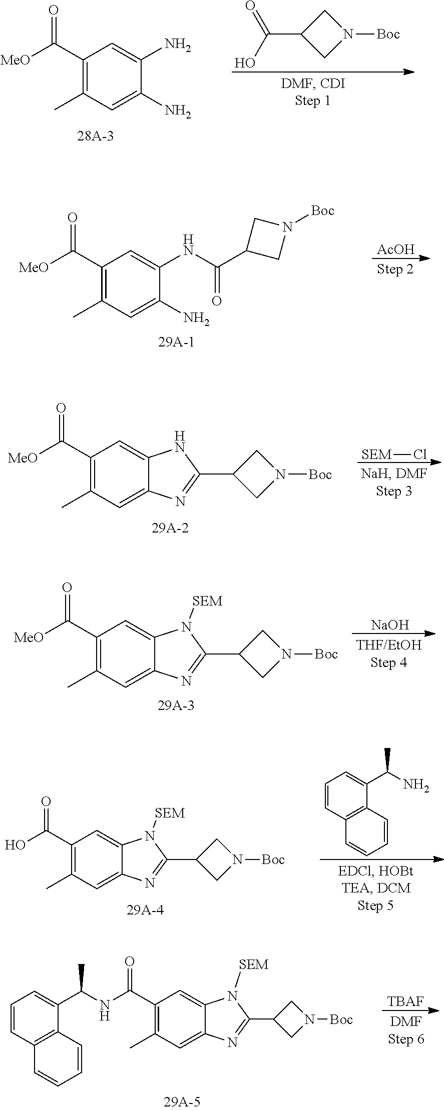

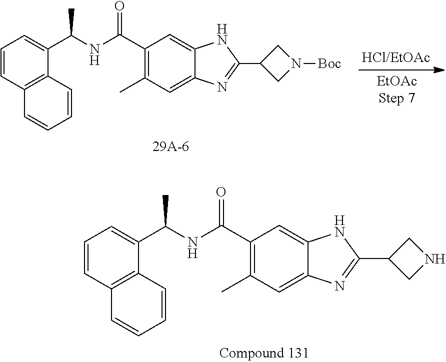 [SMILES: Cc1cc2nc(C3CN(C(=O)OC(C)(C)C)C3)[nH]c2cc1C(=O)N[C@H](C)c1cccc2ccccc12 ; Cc1cc2nc(C3CNC3)[nH]c2cc1C(=O)N[C@H](C)c1cccc2ccccc12]

### Step 1: tert-Butyl 3-((2-amino-5-(methoxycarbonyl)-4-methylphenyl)carbamoyl)azetidine-1-carboxylate (29A-1)

To a solution of methyl 1-(tert-butoxycarbonyl)azetidine-3-carboxylic acid (223 mg, 1.11 mmol, 1.0 eq) in DMF (1.5 mL) were added pyridine (1.5 mL) and CDI (185 mg, 1.11 mmol, 1.0 eq). The mixture was stirred at 45° C. for 30 min. Then to the mixture was added methyl 4,5-diamino-2-methyl-benzoate (200 mg, 1.11 mmol, 1.0 eq), the resulting mixture was stirred at 20° C. for 16 h. LCMS indicated that starting material was completely consumed, and the desired mass was detected. The reaction mixture was poured into H₂O (6.0 mL) and extracted with EtOAc (3.0 mL×3). The combined organic layers were dried over Na₂SO₄, filtered, and concentrated under vacuum to give a residue which was purified by lash silica gel chromatography using a gradient of EtOAc/petroleum ether from 1/100 to 1/1. tert-Butyl 3-((2-amino-5-(methoxycarbonyl)-4-methylphenyl)carbamoyl)azetidine-1-carboxylate (400 mg, 1.10 mmol, 50% yield) was obtained as a white solid. M−100+H⁺=264.3 (LCMS); ¹H NMR (400 MHZ, DMSO-d₆) δ 9.14 (s, 1H), 7.86-7.69 (m, 1H), 6.54 (s, 1H), 5.64 (s, 2H), 3.96 (br s, 4H), 3.71 (s, 3H), 3.54-3.42 (m, 1H), 2.40 (s, 3H), 1.39 (s, 9H).

### Step 2: Methyl 2-(1-(tert-butoxycarbonyl)azetidin-3-yl)-5-methyl-1H-benzo[d]imidazole-6-carboxylate (29A-2)

To a solution of tert-butyl 3-((2-amino-5-(methoxycarbonyl)-4-methylphenyl) carbamoyl)azetidine-1-carboxylate (400 mg, 1.10 mmol, 1.0 eq) in AcOH (0.5 mL) was stirred at 70° C. for 1 h. TLC indicated that the starting material was completely consumed, and a main spot was detected. The reaction mixture was allowed to cool to room temperature, poured into H₂O (5.0 mL) and extracted with EtOAc (2.0 mL×3). The combined organic layers were dried over Na₂SO₄, filtered, and concentrated under vacuum to give a residue which was purified by preparative TLC (EtOAc/petroleum ether=1:1, R_{f}=0.4). Methyl 2-(1-(tert-butoxycarbonyl) azetidin-3-yl)-5-methyl-1H-benzo[d]imidazole-6-carboxy late (350 mg, 1.01 mmol, 92% yield) was obtained as a yellow oil. M+H⁺=346.3 (LCMS).

### Step 3: Methyl 2-(1-(tert-butoxycarbonyl)azetidin-3-yl)-5-methyl-1-((2-(trimethylsilyl)ethoxy)methyl)-1H-benzo[d]imidazole-6-carboxylate (29A-3)

To a solution of methyl 2-(1-(tert-butoxycarbonyl)azetidin-3-yl)-5-methyl-1H-benzo[d]imidazole-6-carboxylate (200 mg, 420 μmol, 1.0 eq) in DMF (6.0 mL) was added sodium hydride (25.2 mg, 631 μmol, 60% purity, 1.5 eq) at 0° C. The mixture was stirred at 0° C. for 30 min. To the mixture was added SEM-Cl (105 mg, 631 μmol, 112 μL, 1.5 eq), the resulting mixture was stirred at 20° C. for 1 h. LCMS indicated that starting material was completely consumed, and the desired mass was detected. The reaction mixture was poured into H₂O (6.0 mL) and extracted with EtOAc (2.0 mL×3). The combined organic layers were dried over Na₂SO₄, filtered, and concentrated under vacuum to give a residue which was purified by preparative TLC (DCM/MeOH=10:1, R_{f}=0.5). Methyl 2-(1-(tert-butoxy carbonyl)azetidin-3-yl)-5-methyl-1-((2-(trimethylsilyl)ethoxy)methyl)-1H-benzo[d]imidazole-6-carboxylate (150 mg, 315 μmol, 75% yield) was obtained as a yellow oil. M+H⁺=476.3 (LCMS).

### Step 4: 2-(1-(tert-Butoxycarbonyl)azetidin-3-yl)-5-methyl-1-((2-(trimethylsilyl)ethoxy)methyl)-1H-benzo[d]imidazole-6-carboxylic acid (29A-4)

To a solution of methyl 2-(1-(tert-butoxycarbonyl)azetidin-3-yl)-5-methyl-1-((2-(trimethylsilyl)ethoxy)methyl)-1H-benzo[d]imidazole-6-carboxylate (80.0 mg, 42.1 μmol, 1.0 eq) in a mixture of EtOH (2.0 mL) and THF (2.0 mL) was added NaOH (2 M aqueous, 2.0 mL, 24 eq). The mixture was stirred at 80° C. for 16 h. LCMS indicated that starting material was completely consumed, and the desired mass was detected. The reaction mixture was allowed to cool to room temperature, poured into H₂O (5.0 mL) and washed with MTBE (3.0 mL×2). The aqueous layer was acidified to pH 5 using HCl (1 M aqueous). The product was extracted with EtOAc (2.0 mL×3). The combined organic layers were dried over Na₂SO₄, filtered, and concentrated under vacuum to give the crude product 2-(1-(tert-butoxycarbonyl)azetidin-3-yl)-5-methyl-1-((2-(trimethylsilyl)ethoxy)methyl)-1H-benzo[d]imidazole-6-carboxylic acid (80.0 mg) as a yellow oil, which was used in the next step without any further purification. M+H⁺=462.4 (LCMS).

### Step 5: (R)-tert-Butyl 3-(5-methyl-6-((1-(naphthalen-1-yl)ethyl)carbamoyl)-1-((2-(trimethylsilyl)ethoxy)methyl)-1H-benzo[d]imidazol-2-yl)azetidine-1-carboxylate (29A-5)

To a solution of 2-(1-(tert-butoxycarbonyl)azetidin-3-yl)-5-methyl-1-((2-(trimethylsilyl) ethoxy)methyl)-1H-benzo[d]imidazole-6-carboxylic acid (70.0 mg, 152 μmol, 1.0 eq) and (R)-1-(naphthalen-1-yl)ethanamine (26.0 mg, 152 μmol, 24.0 μL, 1.0 eq) in DCM (4.0 mL) were added EDCI (34.9 mg, 182 μmol, 1.2 eq), HOBt (24.6 mg, 182 μmol, 1.2 eq) and TEA (46.0 mg, 455 μmol, 63.0 μL, 3.0 eq). The resulting mixture was stirred at 20° C. for 3 h. TLC indicated that the starting material was completely consumed. The reaction mixture was poured into H₂O (5.0 mL) and extracted with DCM (2.0 mL×3). The combined organic layers were dried over Na₂SO₄, filtered, and concentrated under vacuum to give a residue which was purified by preparative TLC (DCM/MeOH=10:1, R_{f}=0.7). (R)-tert-Butyl 3-(5-methyl-6-((1-(naphthalen-1-yl)ethyl)carbamoyl)-1-((2-(trimethylsilyl)ethoxy)methyl)-1H-benzo[d]imidazol-2-yl)azetidine-1-carboxylate (70.0 mg, 114 μmol, 75% yield) was obtained as a yellow solid.

### Step 6: (R)-tert-Butyl 3-(5-methyl-6-((1-(naphthalen-1-yl)ethyl)carbamoyl)-1H-benzo[d]imidazol-2-yl)azetidine-1-carboxylate (29A-6)

To a solution of (R)-tert-butyl 3-(5-methyl-6-((1-(naphthalen-1-yl)ethyl)carbamoyl)-1-((2-(trimethylsilyl)ethoxy)methyl)-1H-benzo[d]imidazol-2-yl)azetidine-1-carboxylate (65.0 mg, 106 μmol, 1.0 eq) in DMF (1.0 mL) were added ethane-1,2-diamine (28.6 mg, 476 μmol, 31.84 μL, 4.5 eq) and TBAF (1 M in THF, 317 μL, 3.0 eq). The resulting mixture was stirred at 20° C. for 3 h. LCMS indicated that the starting material was completely consumed, and the desired mass was detected. The reaction mixture was poured into H₂O (2.0 mL) and extracted with EtOAc (2.0 mL×2). The combined organic layers were dried over Na₂SO₄, filtered, and concentrated under vacuum to give a residue which was purified by preparative TLC (DCM/MeOH=10:1, R_{f}=0.6). (R)-tert-Butyl 3-(5-methyl-6-((1-(naphthalen-1-yl)ethyl) carbamoyl)-1H-benzo[d]imidazol-2-yl)azetidine-1-carboxylate (50.0 mg, 103 μmol, 98% yield) was obtained as a yellow solid. M+H⁺=485.4 (LCMS).

### Step 7: (R)-2-(Azetidin-3-yl)-5-methyl-N-(1-(naphthalen-1-yl)ethyl)-1H-benzo[d]imidazole-6-carboxamide (Compound 131)

To a solution of (R)-tert-butyl 3-(5-methyl-6-((1-(naphthalen-1-yl)ethyl)carbamoyl)-1H-benzo[d]imidazol-2-yl)azetidine-1-carboxylate (50.0 mg, 103 μmol, 1.0 eq) in EtOAc (3.0 mL) was added HCl/EtOAc (4 M, 1.0 mL). The resulting mixture was stirred at 20° C. for 3 h. LCMS indicated that the starting material was completely consumed, and the desired mass was detected. The reaction mixture was concentrated under vacuum to give a residue which was purified by preparative HPLC (Phenomenex Luna C18 column (150×30 mm, 5 μm); flow rate: 25 mL/min; gradient: 1%-40% B over 8 min; mobile phase A: 0.1% aqueous TFA, mobile phase B: acetonitrile). (R)-2-(Azetidin-3-yl)-5-methyl-N-(1-(naphthalen-1-yl)ethyl)-1H-benzo[d]imidazole-6-carboxamide (11.0 mg, 28.5 μmol, 28% yield, TFA salt) was obtained as a white solid. M+H⁺=385.1 (LCMS); ¹H NMR (400 MHZ, CD₃OD) δ 8.93 (br d, J=7.9 Hz, 1H), 8.29 (d, J=8.5 Hz, 1H), 7.92 (d, J=8.6 Hz, 1H), 7.83 (d, J=8.1 Hz, 1H), 7.66-7.43 (m, 6H), 6.18-5.96 (m, 1H), 4.55-4.41 (m, 5H), 2.48 (s, 3H), 1.74 (d, J=7.0 Hz, 3H).

### Example 30: (R)—N-(1-(naphthalen-1-yl)ethyl)-1H-indazole-6-carboxamide (Compound 124)

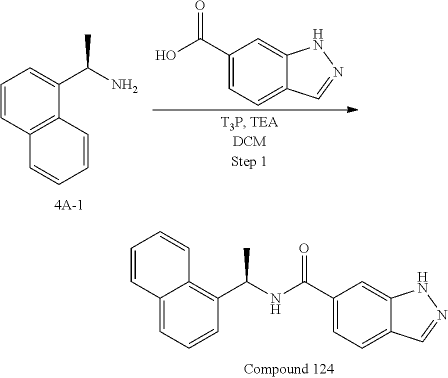 [SMILES: C[C@@H](N)c1cccc2ccccc12 ; O=C(O)c1ccc2cn[nH]c2c1 ; C[C@@H](NC(=O)c1ccc2cn[nH]c2c1)c1cccc2ccccc12]

### Step 1: (R)—N-(1-(naphthalen-1-yl)ethyl)-1H-indazole-6-carboxamide (Compound 124)

To a solution of (R)-1-(naphthalen-1-yl)ethanamine (100 mg, 584 μmol, 1.0 eq) and 1H-indazole-6-carboxylic acid (94.7 mg, 584 μmol, 1.0 eq) in DCM (4.0 mL) were added TEA (118 mg, 1.17 mmol, 163 μL, 2.0 eq), T₃P (2.23 g, 3.50 mmol, 2.08 mL, 50% in EtOAc, 6.0 eq). The mixture was stirred at 20° C. for 3 h. LCMS indicated that the starting material was completely consumed, and the desired mass was detected. The reaction mixture was poured into H₂O (4.0 mL) and extracted with DCM (2.0 mL×2). The combined organic layers were dried over Na₂SO₄, filtered, and concentrated under vacuum to give a residue which was purified by preparative HPLC (Phenomenex Gemini C18 column (100×30 mm, 10 μm); flow rate: 25 mL/min; gradient: 25%-50% B over 8 min; mobile phase A: 10 mM aqueous NH₄HCO₃, mobile phase B: acetonitrile). (R)—N-(1-(Naphthalen-1-yl)ethyl)-1H-indazole-6-carboxamide (28.2 mg, 89.4 μmol, 15% yield) was obtained as a white solid. M+H⁺=316.2 (LCMS); ¹H NMR (400 MHZ, CDCl₃) δ 8.21-8.19 (m, 1H), 8.15-8.09 (m, 1H), 8.03 (s, 1H), 7.93-7.84 (m, 2H), 7.78-7.75 (m, 1H), 7.64-7.61 (m, 1H), 7.55-7.43 (m, 4H), 6.48-6.46 (m, 1H), 6.22-6.14 (m, 1H), 1.84-1.83 (d, J=4, 3H),

### Example 31: (R)-5-Methyl-N-(1-(naphthalen-1-yl)ethyl)-1H-indazole-6-carboxamide (Compound 142)

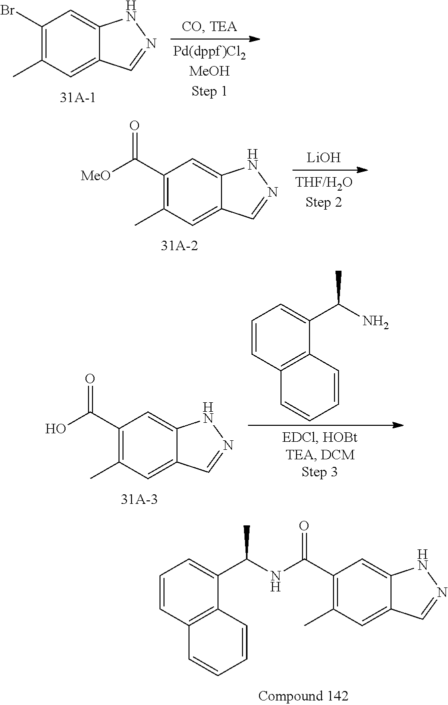

### Step 1: Methyl 5-methyl-1H-indazole-6-carboxylate (31A-2)

To a solution of 6-bromo-5-methyl-1H-indazole (200 mg, 948 μmol, 1.0 eq) in MeOH (3.0 mL) were added Pd(dppf)Cl₂ (139 mg, 190 μmol, 0.2 eq) and TEA (767 mg, 7.58 mmol, 1.10 mL, 8.0 eq) under a N₂ atmosphere. The mixture was degassed and purged with CO three times. The resulting mixture was stirred at 70° C. for 16 h under a CO (50 psi) atmosphere. LCMS indicated that starting material was completely consumed, and the desired mass was detected. The reaction mixture was allowed to cool to room temperature, then concentrated under vacuum to give a residue which was purified by flash silica gel chromatography using a gradient of EtOAc/petroleum ether from 1/100 to 1/10. Methyl 5-methyl-1H-indazole-6-carboxylate (150 mg, 789 μmol, 83% yield). M+H⁺=191.1 (LCMS).

### Step 2: 5-Methyl-1H-indazole-6-carboxylic acid (31A-3)

To a solution of methyl 5-methyl-1H-indazole-6-carboxylate (100 mg, 526 μmol, 1.0 eq) in a mixture of THF (3.0 mL) and H₂O (1.0 mL) was added LiOH (66.2 mg, 1.58 mmol, 3.0 eq). The mixture was stirred at 20° C. for 18 h. LCMS indicated that starting material was completely consumed, and the desired mass was detected. H₂O (5.0 mL) was added and the mixture was washed with MTBE (2.0 mL×3). The aqueous layer was acidified to pH 5 using HCl (1 M aqueous). The product was extracted with EtOAc (2.0 mL×3). The combined organic layers were dried over Na₂SO₄, filtered, and concentrated under vacuum to give 5-methyl-1H-indazole-6-carboxylic acid (70.0 mg, 397 μmol, 76% yield) as a white solid, which was used in the next step without any further purification. M+H⁺=177.2 (LCMS).

### Step 3: (R)-5-Methyl-N-(1-(naphthalen-1-yl)ethyl)-1H-indazole-6-carboxamide (Compound 142)

To a solution of 5-methyl-1H-indazole-6-carboxylic acid (30.0 mg, 170 μmol, 1.0 eq) and (R)-1-(naphthalen-1-yl)ethanamine (29.2 mg, 170 μmol, 27.3 μL, 1.0 eq) in DCM (2.0 mL) were added EDCI (39.2 mg, 204 μmol, 1.2 eq), HOBt (27.6 mg, 204 μmol, 1.2 eq) and TEA (34.5 mg, 341 μmol, 47.4 μL, 2.0 eq). The resulting mixture was stirred at 20° C. for 3 h. LCMS indicated that the starting material was completely consumed, and the desired mass was detected. The reaction mixture was poured into H₂O (5.0 mL) and extracted with DCM (2.0 mL×3). The combined organic layers were dried over Na₂SO₄, filtered, and concentrated under vacuum to give a residue which was purified by preparative HPLC (Phenomenex Gemini C18 column (100×40 mm, 5 μm); flow rate: 25 mL/min; gradient: 45%-90% B over 8 min; mobile phase A: 0.1% aqueous TFA, mobile phase B: acetonitrile). (R)-5-Methyl-N-(1-(naphthalen-1-yl)ethyl)-1H-indazole-6-carboxamide (8.51 mg, 25.8 μmol, 16% yield, TFA salt) was obtained as a white solid. M+H⁺=330.0 (LCMS); ¹H NMR (400 MHZ, CD₃OD) δ 9.00 (br d, J=7.8 Hz, 1H), 8.29 (d, J=8.5 Hz, 1H), 8.10 (br s, 1H), 7.92 (d, J=7.9 Hz, 1H), 7.82 (d, J=8.3 Hz, 1H), 7.66 (d, J=7.1 Hz, 1H), 7.63-7.46 (m, 5H), 6.09 (quin, J=7.1 Hz, 1H), 2.43 (s, 3H), 1.74 (d, J=7.0 Hz, 3H).

### Example 32: (R)-6-Methyl-N-(1-(naphthalen-1-yl)ethyl) quinoline-7-carboxamide (Compound 150)

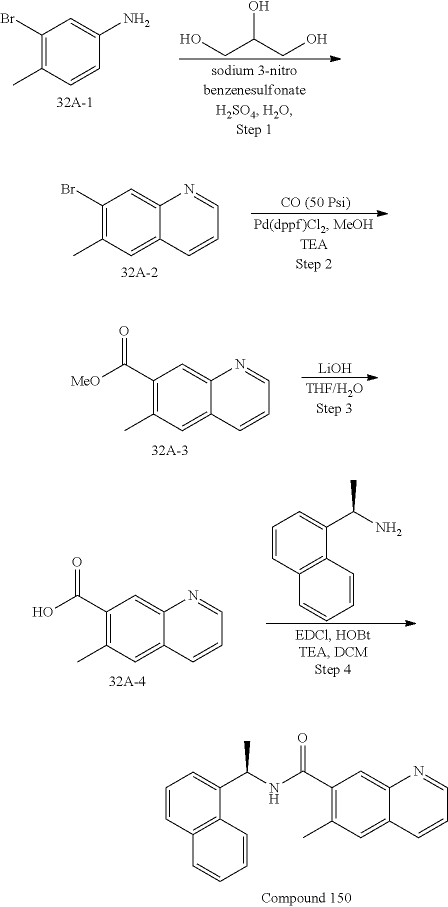

### Step 1: 7-Bromo-6-methylquinoline (32A-2)

To a mixture of 3-bromo-4-methyl-aniline (18.0 g, 96.8 mmol, 1.0 eq) and sodium 3-nitrobenzenesulfonate (26.1 g, 116 mmol, 1.2 eq) in H₂SO₄ (60 mL, 98% purity) and H₂O (25 mL) was added propane-1,2,3-triol (26.7 g, 290 mmol, 21.7 mL, 3.0 eq). The mixture was stirred at 130° C. for 2 h. TLC indicated that that the starting material was completely consumed. The reaction mixture was allowed to cool to room temperature, poured into H₂O (30 mL) and neutralized with K₂CO₃ to pH 8. The product was extracted with EtOAc (30 mL×5). The combined organic layers were dried over Na₂SO₄, filtered, and concentrated under vacuum to give a residue which was purified by flash silica gel chromatography using a gradient of EtOAc/petroleum ether from 0/1 to 1/2. 7-Bromo-6-methylquinoline (10.4 g, 46.8 mmol, 48% yield) was obtained as a white solid. ¹H NMR (400 MHZ, CDCl₃) δ 8.87-8.85 (m, 1H), 8.63-8.56 (m, 1H), 8.01-7.96 (m, 1H), 7.62-7.58 (m, 1H), 7.41-7.35 (m, 1H), 2.60-2.56 (m, 3H).

### Step 2: Methyl 6-methylquinoline-7-carboxylate (32A-3)

To a solution of 7-bromo-6-methylquinoline (6.00 g, 27.0 mmol, 1.0 eq) and TEA (10.9 g, 108 mmol, 15.0 mL, 4.0 eq) in MeOH (50 mL) was added Pd(dppf)Cl₂ (1.98 g, 2.70 mmol, 0.1 eq). The mixture was stirred at 70° C. for 16 h under a CO (50 psi) atmosphere. TLC indicated that the starting material was completely consumed. The reaction mixture was concentrated under vacuum to give a residue which was purified by flash silica gel chromatography using a gradient of EtOAc/petroleum ether from 0/1 to 1/2. Methyl 6-methylquinoline-7-carboxylate (6.00 g, 29.8 mmol, 37% yield) was obtained as a white solid. ¹H NMR (400 MHZ, CDCl₃) δ 8.98-8.89 (m, 1H), 8.72-8.64 (m, 1H), 8.14-8.06 (m, 1H), 7.71-7.63 (m, 1H), 7.49-7.39 (m, 1H), 4.16-3.89 (m, 3H), 2.85-2.68 (m, 3H).

### Step 3: 6-Methylquinoline-7-carboxylic acid (32A-4)

To a solution of methyl 6-methylquinoline-7-carboxylate (300 mg, 1.49 mmol, 1.0 eq) in a mixture of THF (8.0 mL), H₂O (4.0 mL) and MeOH (2.0 mL) was added LiOH·H₂O (125 mg, 2.98 mmol, 2.0 eq). The mixture was stirred at 20° C. for 30 min, then at 70° C. for 16 h. LCMS indicated that the starting material was completely consumed, and the desired mass was detected. The reaction mixture was allowed to cool to room temperature, poured into H₂O (8.0 mL) and washed with MTBE (8.0 mL×2). The aqueous layer was acidified to pH 5 with HCl (1 M aqueous). The product was extracted with EtOAc (8.0 mL×6). The combined organic layers were dried over Na₂SO₄, filtered, and concentrated under vacuum to give 6-methylquinoline-7-carboxylic acid (270 mg, 1.44 mmol, 97% yield) as a white solid, which was used in the next step without any further purification. M−H⁻=186.1 (LCMS).

### Step 4: (R)-6-Methyl-N-(1-(naphthalen-1-yl)ethyl)quinoline-7-carboxamide (Compound 150)

To a solution of (R)-1-(naphthalen-1-yl)ethanamine (89.2 mg, 521 μmol, 83.0 μL, 1.5 eq) and 6-methylquinoline-7-carboxylic acid (65.0 mg, 347 μmol, 1.0 eq) in DCM (5.0 mL) were added TEA (70.3 mg, 694 μmol, 96.7 μL, 2.0 eq) and T₃P (331 mg, 521 μmol, 310 μL, 50% purity in EtOAc, 1.5 eq). The mixture was stirred at 20° C. for 16 h. LCMS indicated that the starting material was completely consumed, and the desired mass was detected. The reaction mixture was poured into H₂O (10 mL) and extracted with DCM (10 mL×2). The combined organic layers were dried over Na₂SO₄, filtered, and concentrated under vacuum to give a residue which was purified by was purified by preparative TLC (EtOAc/petroleum ether=1/3, R_{f}=0.5). (R)-6-Methyl-N-(1-(naphthalen-1-yl)ethyl) quinoline-7-carboxamide (26.0 mg, 76.4 μmol, 22% yield) was obtained as a white solid. M+H⁺=341.1 (LCMS); ¹H NMR (400 MHZ, CDCl₃) δ 8.82 (br d, J=2.1 Hz, 1H), 8.32-8.23 (m, 1H), 8.12-7.98 (m, 2H), 7.90 (d, J=8.0 Hz, 1H), 7.86-7.79 (m, 1H), 7.67-7.57 (m, 3H), 7.57-7.44 (m, 2H), 7.44-7.36 (m, 1H), 6.36-6.14 (m, 2H), 2.69-2.59 (m, 3H), 1.90-1.80 (m, 3H).

### Example 33: (R)-6-Methyl-N-(1-(naphthalen-1-yl)ethyl)-2-oxo-1,2-dihydroquinoline-7-carboxamide (Compound 167)

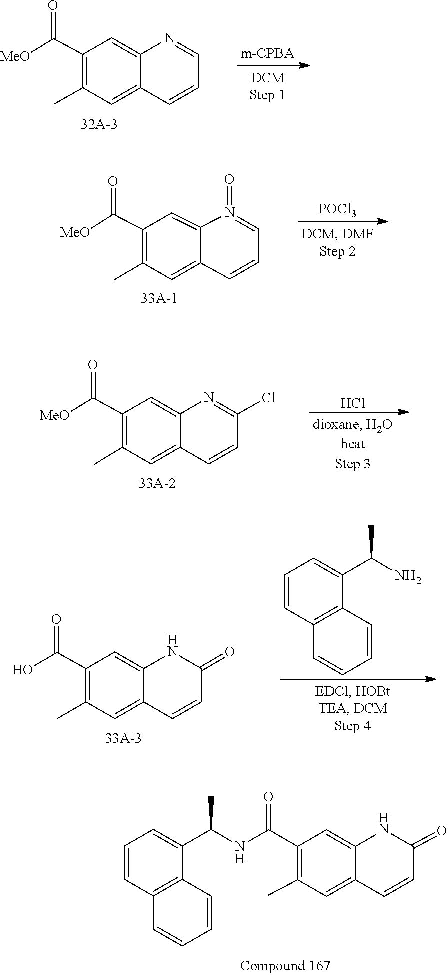

### Step 1: 7-(Methoxycarbonyl)-6-methylquinoline 1-oxide (33A-1)

To a mixture of methyl 6-methylquinoline-7-carboxylate (240 mg, 1.19 mmol, 1.0 eq) in DCM (5.0 mL) was added m-CPBA (308 mg, 1.43 mmol, 80% purity, 1.2 eq) at 0° C. The mixture was stirred at 20° C. for 2 h under a N₂ atmosphere. LCMS indicated that the starting material was completely consumed, and the desired mass was detected. The reaction was poured into saturated aqueous Na₂SO₃ (10 mL) and extracted with DCM (5.0 mL×3). The combined organic layers were dried and concentrated under vacuum to give 7-(methoxycarbonyl)-6-methylquinoline 1-oxide (300 mg, 968 μmol, 81% yield) as a yellow solid. M+H⁺=218.1 (LCMS); ¹H NMR (400 MHZ, CDCl₃) δ 9.33-9.23 (m, 1H), 8.56-8.46 (m, 1H), 7.80-7.70 (m, 1H), 7.69-7.60 (m, 1H), 7.38-7.30 (m, 1H), 4.04-3.91 (m, 3H), 2.82-2.70 (m, 3H).

### Step 2: Methyl 2-chloro-6-methylquinoline-7-carboxylate (33A-2)

To a solution of 7-(methoxycarbonyl)-6-methylquinoline 1-oxide (230 mg, 1.06 mmol, 1.0 eq) in DCM (8.0 mL) was added POCl₃ (244 mg, 1.59 mmol, 148 μL, 1.5 eq) at 0° C., followed by DMF (39.0 mg, 529 μmol, 40.7 L, 0.5 eq). The mixture was stirred at 20° C. for 16 h. LCMS indicated that the starting material was completely consumed, and the desired mass was detected. Saturated aqueous Na₂CO₃ solution was added to the reaction mixture slowly at 0° C. to adjust the pH to 8. The resulting mixture was separated and the aqueous phase was extracted with DCM (10 mL×5). The combined organic layers were washed with brine (10 mL), dried over Na₂SO₄, and concentrated under vacuum to afford the crude product which was purified by flash silica gel chromatography using a gradient of EtOAc/petroleum ether from 0/1 to 1/2. Methyl 2-chloro-6-methylquinoline-7-carboxylate (120 mg, 509 μmol, 48% yield) was obtained as a white solid. M+H⁺=236.0 (LCMS); ¹H NMR (400 MHZ, CDCl₃) δ 8.69-8.62 (m, 1H), 8.17-7.96 (m, 1H), 7.77-7.59 (m, 1H), 7.47 (d, J=8.5 Hz, 1H), 3.98 (s, 3H), 2.80-2.70 (m, 3H).

### Step 3: 6-Methyl-2-oxo-1,2-dihydroquinoline-7-carboxylic acid (33A-3)

To a solution of methyl 2-chloro-6-methylquinoline-7-carboxylate (120 mg, 509 μmol, 1.0 eq) in dioxane (5.0 mL) was added HCl (6 M in aqueous, 962 μL, 11 eq). The mixture was stirred at 100° C. for 16 h. LCMS indicated that the starting material was completely consumed, and the desired mass was detected. The reaction mixture was allowed to cool to room temperature, diluted with H₂O (3.0 mL) and acidified to pH 5 with NaOH (2 M aqueous). The product was extracted with EtOAc (8.0 mL×5). The combined organic layers were dried over Na₂SO₄, filtered, and concentrated under vacuum to give the product 6-methyl-2-oxo-1,2-dihydroquinoline-7-carboxylic acid (160 mg, 472 μmol, 93% yield) as a white solid, which was used in the next step without any further purification. M+H⁺=204.0 (LCMS); ¹H NMR (400 MHZ, DMSO-d₆) δ 11.83-11.74 (m, 1H), 7.91-7.83 (m, 1H), 7.81-7.76 (m, 1H), 7.58-7.54 (m, 1H), 6.63-6.52 (m, 1H), 1.25-1.20 (m, 3H).

### Step 4: (R)-6-Methyl-N-(1-(naphthalen-1-yl)ethyl)-2-oxo-1,2-dihydroquinoline-7-carboxamide (Compound 167)

To a solution of (R)-1-(naphthalen-1-yl)ethanamine (75.8 mg, 443 μmol, 70.9 μL, 1.5 eq) and 6-methyl-2-oxo-1,2-dihydroquinoline-7-carboxylic acid (100 mg, 295 μmol, 1.0 eq) in DCM (10 mL) were added TEA (89.6 mg, 886 μmol, 123 μL, 3.0 eq), EDCI (84.9 mg, 443 μmol, 1.5 eq) and HOBt (59.9 mg, 442 μmol, 1.5 eq). The mixture was stirred at 20° C. for 16 h. LCMS indicated that the starting material was completely consumed, and the desired mass was detected. The reaction mixture was poured into H₂O (10 mL) and extracted with DCM (10 mL×5). The combined organic layers were dried over Na₂SO₄, filtered, and concentrated under vacuum to give a residue which was purified by preparative HPLC (Phenomenex Gemini C18 column (75×30 mm, 3 μm); flow rate: 25 mL/min; gradient: 25%-65% B over 8 min; mobile phase A: 0.04% aqueous HCl, mobile phase B: acetonitrile). (R)-6-Methyl-N-(1-(naphthalen-1-yl)ethyl)-2-oxo-1,2-dihydroquinoline-7-carboxamide (8.00 mg, 21.9 μmol, 7% yield) was obtained as a white solid. M+H⁺=357.1 (LCMS); ¹H NMR (400 MHZ, DMSO-d₆) δ 11.84-11.57 (m, 1H), 9.13-8.98 (m, 1H), 8.37-8.13 (m, 1H), 8.00-7.93 (m, 1H), 7.91-7.80 (m, 2H), 7.65-7.47 (m, 5H), 7.29-7.21 (m, 1H), 6.54-6.44 (m, 1H), 5.98-5.87 (m, 1H), 2.30-2.24 (m, 3H), 1.62-1.53 (m, 3H).

### Example 34: (R)-6-Methyl-N-(1-(naphthalen-1-yl)ethyl)benzo[d][1,3]dioxole-5-carboxamide (Compound 145)

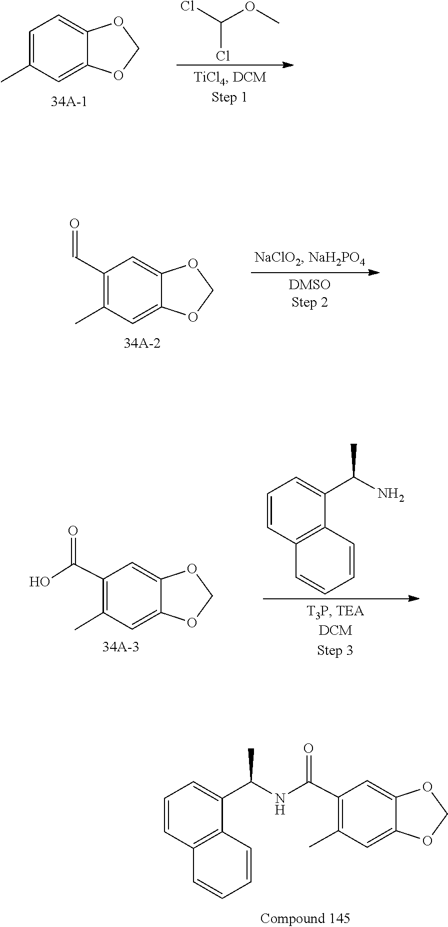

### Step 1: 6-Methylbenzo[d][1,3]dioxole-5-carbaldehyde (34A-2)

To a solution of 5-methylbenzo[d][1,3]dioxole (200 mg, 1.47 mmol, 175 μL, 1.0 eq) in DCM (2.0 mL) was added dichloro(methoxy) methane (338 mg, 2.94 mmol, 260 μL, 2.0 eq) at 0° C. The mixture was stirred at 0° C. for 15 min and a solution of TiCl₄ (334 mg, 1.76 mmol, 1.2 eq) in DCM (2.0 mL) was added dropwise. The resulting mixture was stirred at 20° C. for 1 h. TLC indicated that the starting material was completely consumed. The mixture was poured into H₂O (10 mL) and extracted with MTBE (10 mL×2). The combined organic layers were dried and concentrated in vacuum to give a residue which was purified by flash silica gel chromatography using a gradient of EtOAc/petroleum ether from 0/100 to 1/50. 6-Methylbenzo[d][1,3]dioxole-5-carbaldehyde (220 mg, 1.34 mmol, 91% yield) was obtained as a yellow solid. ¹H NMR (400 MHZ, CDCl₃) δ 10.24-10.05 (m, 1H), 7.29 (s, 1H), 6.70 (s, 1H), 6.03 (s, 2H), 2.62 (s, 3H).

### Step 2: 6-Methylbenzo[d][1,3]dioxole-5-carboxylic acid (34A-3)

To a stirred solution of 6-methylbenzo[d][1,3]dioxole-5-carbaldehyde (200 mg, 1.22 mmol, 1.0 eq), NaH₂PO₄ (43.9 mg, 366 μmol, 0.3 eq), and H₂O₂ (177 mg, 1.82 mmol, 150 μL, 35% purity in H₂O, 1.5 eq) in a mixture of acetonitrile (1.5 mL) and H₂O (0.6 mL) was added a solution of NaClO₂ (154 mg, 1.71 mmol, 1.4 eq) in H₂O (2.0 mL) dropwise to maintain the temperature between 0° C. and 10° C. The resulting mixture was stirred at 20° C. for 1.5 h. TLC indicated that the starting material was completely consumed. The reaction mixture was poured into saturated aqueous Na₂SO₃ (10 mL) and extracted with DCM (5.0 mL×3). The combined organic layers were dried over Na₂SO₄, filtered, and concentrated under vacuum to give crude 6-methylbenzo[d][1,3]dioxole-5-carboxylic acid (220 mg) as a white solid.

### Step 3: (R)-6-Methyl-N-(1-(naphthalen-1-yl)ethyl)benzo[d][1,3]dioxole-5-carboxamide (Compound 145)

To a stirred solution of 6-methylbenzo[d][1,3]dioxole-5-carboxylic acid (61.0 mg, 339 μmol, 1.0 eq) and (R)-1-(naphthalen-1-yl)ethanamine (63.8 mg, 372 μmol, 59.6 μL, 1.1 eq) in DCM (5.0 mL) were added TEA (103 mg, 1.02 mmol, 141 μL, 3.0 eq) and T₃P (323 mg, 508 μmol, 302 μL, 50% purity in EtOAc, 1.5 eq). The mixture was stirred at 20° C. for 2 h. LCMS indicated that the starting material was completely consumed, and the desired compound was detected. The reaction mixture was poured into H₂O (2.0 mL) and extracted with DCM (3.0 mL×2). The combined organic layers were dried over Na₂SO₄, filtered, and concentrated under vacuum to give a residue which was purified by preparative TLC (EtOAc/petroleum ether=1/3, R_{f}=0.3). (R)-6-Methyl-N-(1-(naphthalen-1-yl)ethyl)benzo[d][1,3]dioxole-5-carboxamide (37.5 mg, 106 μmol, 31% yield) was obtained as a white solid. M+H⁺=334.0 (LCMS); 1H NMR (400 MHz, CDCl₃) δ 8.30-8.18 (m, 1H), 7.92-7.86 (m, 1H), 7.85-7.80 (m, 1H), 7.63-7.44 (m, 4H), 6.81-6.75 (m, 1H), 6.68-6.60 (m, 1H), 6.17-6.05 (m, 1H), 5.94-5.84 (m, 3H), 2.41-2.32 (m, 3H), 1.83-1.74 (m, 3H).

### Example 35: N-(5-Amino-2-methylphenyl)-2-(naphthalen-1-yl)propanamide (Compound 163)

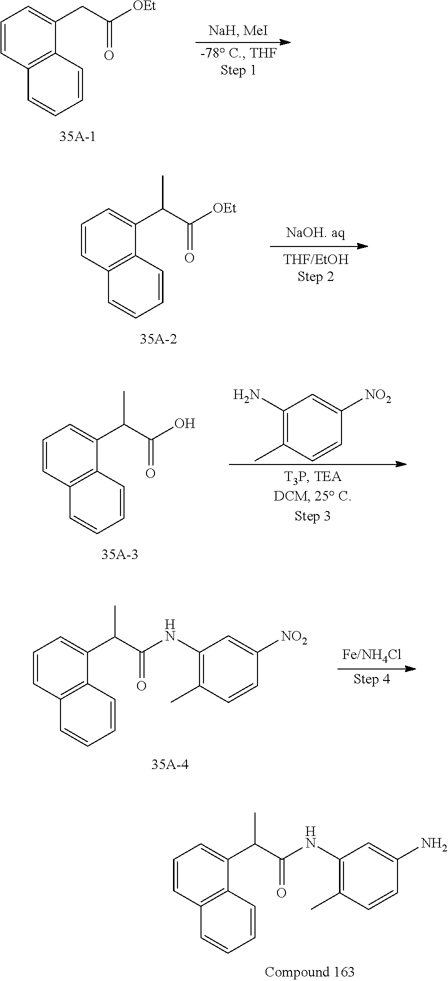 [SMILES: CCOC(=O)Cc1cccc2ccccc12 ; CCOC(=O)C(C)c1cccc2ccccc12 ; CC(C(=O)O)c1cccc2ccccc12 ; Cc1ccc([N+](=O)[O-])cc1N ; Cc1ccc([N+](=O)[O-])cc1NC(=O)C(C)c1cccc2ccccc12 ; Cc1ccc(N)cc1NC(=O)C(C)c1cccc2ccccc12]

### Step 1: Ethyl 2-(naphthalen-1-yl)propanoate (35A-2)

To a stirred solution of ethyl 2-(naphthalen-1-yl)acetate (500 mg, 2.33 mmol, 1.0 eq) in DMF (10 mL) was added sodium hydride (112 mg, 2.80 mmol, 60% purity, 1.2 eq) in portions at 0° C. The mixture was stirred at the same temperature for 15 min. Iodomethane (364 mg, 2.57 mmol, 160 μL, 1.1 eq) was added dropwise. After the addition was complete, the resulting mixture was stirred at 0° C. for another 15 min. TLC indicated that that the starting material was completely consumed. The mixture was poured into H₂O (20 mL) and extracted with MTBE (15 mL×3). The combined organic layers were dried over Na₂SO₄, filtered, and concentrated under vacuum to give ethyl 2-(naphthalen-1-yl)propanoate (450 mg, 1.97 mmol, 84% yield) as a yellow oil, which was used in the next step without any further purification.

### Step 2: 2-(Naphthalen-1-yl)propanoic acid (35A-3)

To a stirred solution of ethyl 2-(naphthalen-1-yl)propanoate (200 mg, 876 μmol, 1.0 eq) in a mixture of EtOH (5.0 mL) and THF (5.0 mL) was added NaOH (2 M aqueous, 1.31 mL, 3.0 eq). The mixture was stirred at 20° C. for 1 h and then at 70° C. for 5 h. TLC indicated that the starting material was completely consumed. The reaction mixture was allowed to cool to room temperature, poured into water (20 mL) and washed with MTBE (15 mL×3). The aqueous was acidified to pH 5 using HCl (1 M aqueous) and extracted with EtOAc (5 mL×2). The combined organic layers were dried and concentrated in vacuum to give 2-(naphthalen-1-yl)propanoic acid (180 mg, 90% purity) as a yellow solid. ¹H NMR (400 MHZ, CDCl₃) δ 8.22-8.05 (m, 1H), 7.94-7.87 (m, 1H), 7.85-7.79 (m, 1H), 7.61-7.45 (m, 4H), 4.64-4.52 (m, 1H), 1.74-1.68 (m, 3H).

### Step 3: N-(2-Methyl-5-nitrophenyl)-2-(naphthalen-1-yl)propanamide (35A-4)

To a stirred solution of 2-(naphthalen-1-yl)propanoic acid (100 mg, 499 μmol, 1.0 eq) and 2-methyl-5-nitroaniline (83.6 mg, 549 μmol, 209 μL, 1.1 eq) in DCM (5.0 mL) was added TEA (152 mg, 1.50 mmol, 209 μL, 3.0 eq), followed by T₃P (636 mg, 999 μmol, 594 μL, 50% purity in EtOAc, 2.0 eq). The mixture was stirred at 20° C. for 16 h. LCMS indicated that the starting material was completely consumed, and the desired compound was detected. The mixture was poured into H₂O (10 mL) and extracted with MTBE (10 mL×3). The combined organic layers were dried over Na₂SO₄, filtered, and concentrated under vacuum to give a residue which was purified via preparative TLC (EtOAc/petroleum ether=1/3, R_{f}=0.3). N-(2-Methyl-5-nitrophenyl)-2-(naphthalen-1-yl)propanamide (90.0 mg, 269 μmol, 54% yield) was obtained as a yellow solid. M+H⁺=335.1 (LCMS).

### Step 4: N-(5-Amino-2-methylphenyl)-2-(naphthalen-1-yl)propanamide (Compound 163)

To a stirred solution of N-(2-methyl-5-nitrophenyl)-2-(naphthalen-1-yl)propanamide (60.0 mg, 179 μmol, 1.0 eq) in a mixture of MeOH (10 mL) and H₂O (2.5 mL) was added iron powder (50.1 mg, 897 μmol, 5.0 eq), followed by NH₄Cl (48.0 mg, 897 μmol, 5.0 eq). The mixture was stirred at 80° C. for 16 h. LCMS indicated that the starting material was completely consumed, and the desired mass was detected. The reaction mixture was allowed to cool to room temperature, poured into H₂O (20 mL) and extracted with DCM (10 mL×3). The combined organic layers were dried over Na₂SO₄, filtered, and concentrated under vacuum to give the crude product which was purified by preparative HPLC (Waters Xbridge Prep OBD C18 column (150×40 mm, 10 μm); flow rate: 60 mL/min; gradient: 30%-60% B over 8 min; mobile phase A: 10 mM aqueous NH₄HCO₃, mobile phase B: acetonitrile). N-(5-Amino-2-methylphenyl)-2-(naphthalen-1-yl)propanamide (8.11 mg, 26.4 μmol, 15% yield) was obtained as a yellow solid. M+H⁺=305.0 (LCMS); ¹H NMR (400 MHZ, CD₃OD) δ 8.32-8.20 (m, 1H), 7.93-7.88 (m, 1H), 7.85-7.79 (m, 1H), 7.66-7.62 (m, 1H), 7.61-7.54 (m, 1H), 7.54-7.46 (m, 2H), 6.90-6.84 (m, 1H), 6.75-6.70 (m, 1H), 6.53-6.47 (m, 1H), 4.73-4.62 (m, 1H), 1.87-1.82 (m, 3H), 1.77-1.63 (m, 3H).

### Example 36: 5-Amino-N-(cyano(naphthalen-1-yl)methyl)-2-methylbenzamide (Compound 195)

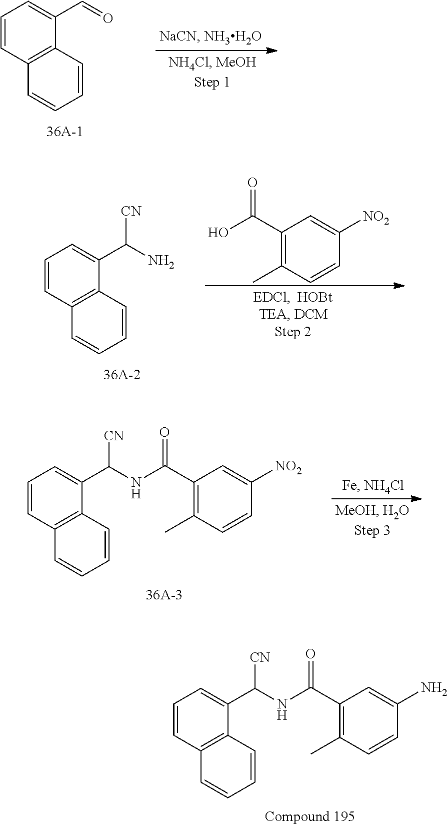 [SMILES: O=Cc1cccc2ccccc12 ; N#CC(N)c1cccc2ccccc12 ; Cc1ccc([N+](=O)[O-])cc1C(=O)O ; Cc1ccc([N+](=O)[O-])cc1C(=O)NC(C#N)c1cccc2ccccc12 ; Cc1ccc(N)cc1C(=O)NC(C#N)c1cccc2ccccc12]

### Step 1: 2-Amino-2-(naphthalen-1-yl) acetonitrile (36A-2)

To a solution of sodium cyanide (158 mg, 3.20 mmol, 1.0 eq) in MeOH (3.0 mL) was NH₃·H₂O (1.82 g, 13.1 mmol, 2.00 mL, 25% purity, 4.1 eq), NH₄Cl (171 mg, 3.20 mmol, 1.0 eq) and 1-naphthaldehyde (500 mg, 3.20 mmol, 435 μL, 1.0 eq). The mixture was stirred at 20° C. for 5 h. LCMS indicated that the starting material was completely consumed, and the desired mass was detected. The reaction mixture was poured into H₂O (4.0 mL) and extracted with EtOAc (3.0 mL×2). The combined organic layers were concentrated under vacuum to give the crude product 2-amino-2-(naphthalen-1-yl) acetonitrile (450 mg), which was used in the next step without any further purification. M−17+H⁺=166.1 (LCMS).

### Step 2: N-(Cyano(naphthalen-1-yl)methyl)-2-methyl-5-nitrobenzamide (36A-3)

To a solution of 2-amino-2-(naphthalen-1-yl) acetonitrile (100 mg, 549 μmol, 1.0 eq) and 2-methyl-5-nitrobenzoic acid (99.4 mg, 549 μmol, 1.0 eq) in DCM (3.0 mL) were added TEA (166 mg, 1.65 mmol, 229 μL, 3.0 eq), EDCI (263 mg, 1.37 mmol, 2.5 eq) and HOBt (185 mg, 1.37 mmol, 2.5 eq). The mixture was stirred at 20° C. for 16 h. LCMS indicated that the starting material was completely consumed, and the desired mass was detected. The reaction mixture was poured into H₂O (3.0 mL) at 25° C. and extracted with EtOAc (1.0 mL×3). The combined organic layers were dried over Na₂SO₄, filtered, and concentrated under vacuum to give the crude product N-(cyano(naphthalen-1-yl)methyl)-2-methyl-5-nitrobenzamide (170 mg), which was used in the next step without any further purification. M+H⁺=346.1 (LCMS).

### Step 3: 5-Amino-N-(cyano(naphthalen-1-yl)methyl)-2-methylbenzamide (Compound 195)

To a solution of N-(cyano(naphthalen-1-yl)methyl)-2-methyl-5-nitrobenzamide (170 mg, 492 μmol, 1.0 eq) in a mixture of MeOH (6.0 mL) and H₂O (2.0 mL) were added iron powder (137 mg, 2.46 mmol, 5.0 eq) and NH₄Cl (132 mg, 2.46 mmol, 5.0 eq). The mixture was stirred at 80° C. for 2 h. LCMS indicated that the starting material was completely consumed, and the desired mass was detected. The reaction mixture was allowed to cool to room temperature, poured into H₂O (8.0 mL) at 25° C. and extracted with EtOAc (3.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 20%-40% B over 8 min; mobile phase A: 0.04% aqueous HCl, mobile phase B: acetonitrile). 5-Amino-N-(cyano(naphthalen-1-yl)methyl)-2-methylbenzamide (33.3 mg, 102 μmol, 21% yield, HCl salt) was obtained as a white solid. M+H⁺=316.2 (LCMS); ¹H NMR (400 MHZ, DMSO-d₆) δ 9.73 (d, J=8.0 Hz, 1H), 8.09-8.01 (m, 3H), 7.85 (d, J=7.1 Hz, 1H), 7.69-7.57 (m, 2H), 7.56-7.49 (m, 1H), 7.70-7.46 (m, 1H), 7.15-7.02 (m, 2H), 6.97-6.86 (m, 2H), 2.24-2.17 (m, 3H).

### Example 37: 5-Amino-N-(2-fluoro-1-(naphthalen-1-yl)ethyl)-2-methylbenzamide (Compound 200)

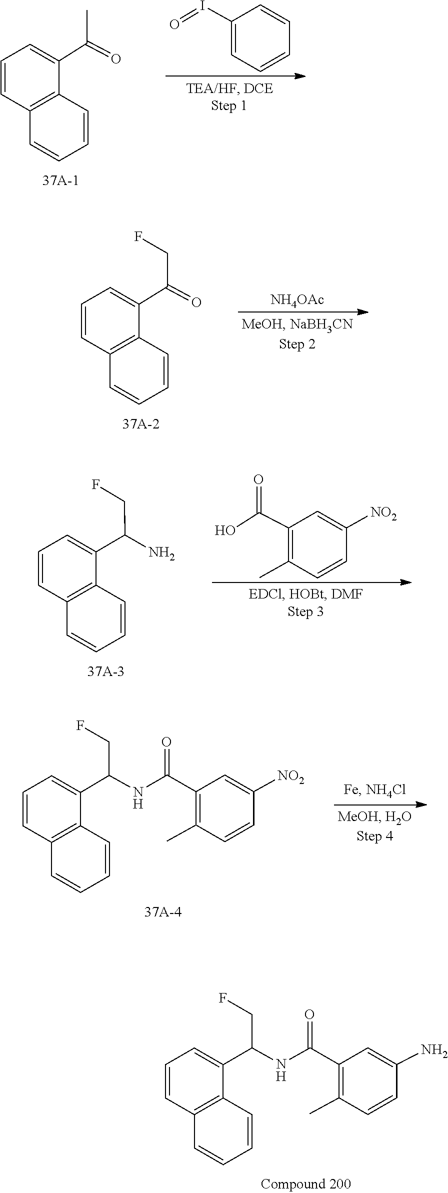

### Step 1: 2-Fluoro-1-(naphthalen-1-yl)ethanone (37A-2)

To a solution of iodosylbenzene (1.55 g, 7.05 mmol, 1.2 eq) and hydrogen fluoride (4.07 g, 23.5 mmol, 8.66 mL, 70% purity in TEA, 4.0 eq) in DCE (10 mL) was added 1-(naphthalen-1-yl)ethanone (1.00 g, 5.88 mmol, 1.0 eq). The mixture was stirred at 70° C. for 16 h. LCMS indicated that the starting material was completely consumed, and the desired mass was detected. The reaction mixture was allowed to cool to room temperature, poured into saturated aqueous NaHCO₃ (20 mL) and extracted with DCM (10 mL×3). The combined organic layers were washed with brine (20 mL), dried over Na₂SO₄, filtered, and concentrated under vacuum to give a residue which was purified by flash silica gel chromatography using a gradient of EtOAc/petroleum ether from 0/1 to 1/1. 2-Fluoro-1-(naphthalen-1-yl)ethanone (270 mg, 1.43 mmol, 24% yield) was obtained as a yellow solid. M+H⁺=189.1 (LCMS).

### Step 2: 2-Fluoro-1-(naphthalen-1-yl)ethanamine (37A-3)

To a solution of 2-fluoro-1-(naphthalen-1-yl)ethenone (130 mg, 691 μmol, 1.0 eq) in MeOH (15 mL) were added NH₄OAc (1.28 g, 16.6 mmol, 24 eq) and NaBH₃CN (347 mg, 5.53 mmol, 8.0 eq). The mixture was stirred at 60° C. for 16 h. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by preparative TLC (EtOAc/petroleum ether=1/3, R_{f}=0.1). 2-Fluoro-1-(naphthalen-1-yl)ethanamine (150 mg, crude) was obtained as a yellow oil. M+H⁺=190.2 (LCMS).

### Step 3: N-(2-Fluoro-1-(naphthalen-1-yl)ethyl)-2-methyl-5-nitrobenzamide (37A-4)

To a solution of 2-fluoro-1-(naphthalen-1-yl)ethanamine (273 mg, 1.44 mmol, 1.0 eq) and 2-methyl-5-nitrobenzoic acid (287 mg, 1.59 mmol, 1.1 eq) in DMF (10 mL) were added TEA (438 mg, 4.33 mmol, 602 μL, 3.0 eq), EDCI (691 mg, 3.61 mmol, 2.5 eq) and HOBt (487 mg, 3.61 mmol, 2.5 eq). The mixture was stirred at 20° C. for 16 h. TLC indicated that the starting material was completely consumed. The reaction mixture was poured into H₂O (10 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. N-(2-Fluoro-1-(naphthalen-1-yl)ethyl)-2-methyl-5-nitrobenzamide (180 mg, 511 μmol, 35% yield) was obtained as a brown solid. M+H⁺=353.2 (LCMS); ¹H NMR (400 MHZ, CDCl₃) δ 8.23 (d, J=2.0 Hz, 1H), 8.21-8.09 (m, 2H), 7.90 (dd, J=8.1, 15.6 Hz, 2H), 7.67-7.60 (m, 2H), 7.57 (br d, J=7.7 Hz, 1H), 7.51 (d, J=7.8 Hz, 1H), 7.39 (d, J=8.4 Hz, 1H), 6.51 (br d, J=8.2 Hz, 1H), 6.42-6.21 (m, 1H), 5.19-4.85 (m, 2H), 2.55 (s, 3H).

### Step 4: 5-Amino-N-(2-fluoro-1-(naphthalen-1-yl)ethyl)-2-methylbenzamide (Compound 200)

To a solution of N-(2-fluoro-1-(naphthalen-1-yl)ethyl)-2-methyl-5-nitrobenzamide (100 mg, 284 μmol, 1.0 eq) in a mixture of MeOH (10 mL) and H₂O (2.5 mL) were added iron powder (79.2 mg, 1.42 mmol, 5.0 eq) and NH₄Cl (75.9 mg, 1.42 mmol, 5.0 eq). The mixture was stirred at 80° C. for 16 h. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 25%-55% B over 8 min; mobile phase A: 10 mM aqueous NH₄HCO₃, mobile phase B: acetonitrile). 5-Amino-N-(2-fluoro-1-(naphthalen-1-yl)ethyl)-2-methyl benzamide (26.8 mg, 83.0 μmol, 29% yield) was obtained as a white solid. M+H⁺=323.1 (LCMS); ¹H NMR (400 MHZ, CDCl₃) δ 8.18 (d, J=8.6 Hz, 1H), 7.89 (dd, J=8.1, 17.4 Hz, 2H), 7.67-7.45 (m, 4H), 6.99 (d, J=8.1 Hz, 1H), 6.71 (s, 1H), 6.65 (br d, J=7.7 Hz, 1H), 6.37-6.18 (m, 2H), 5.13-4.85 (m, 2H), 2.33 (s, 3H).

### Example 38: 5-Amino-N-(2,2-difluoro-1-(naphthalen-1-yl)ethyl)-2-methylbenzamide (Compound 185)

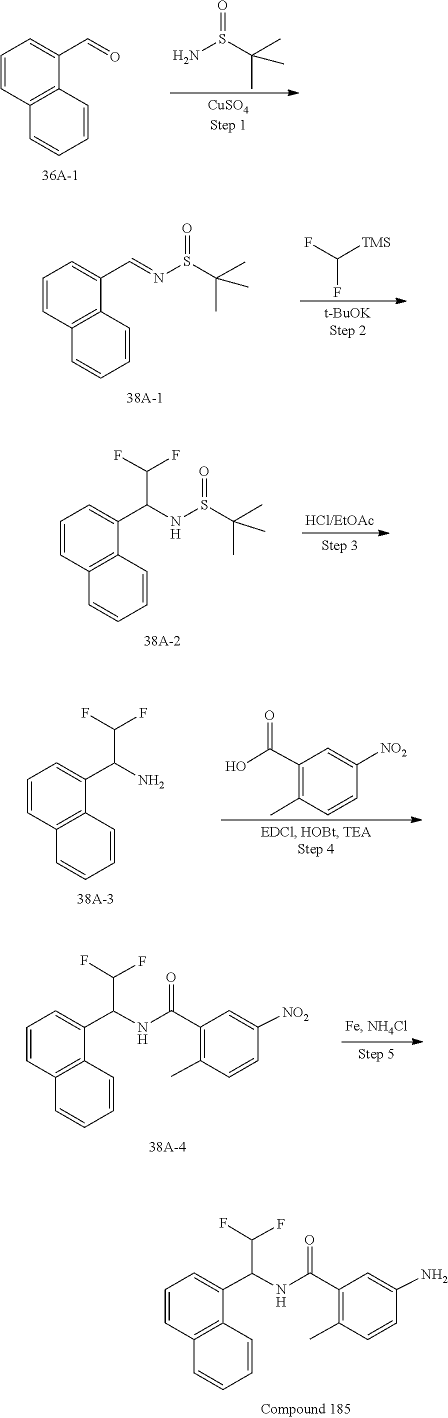

### Step 1: (E)-2-Methyl-N-(naphthalen-1-ylmethylene) propane-2-sulfinamide (38A-1)

To a solution of 2-methylpropane-2-sulfinamide (1.55 g, 12.8 mmol, 1.0 eq) in DCM (25 mL) were added CuSO₄ (4.09 g, 25.6 mmol, 3.93 mL, 2.0 eq) and 1-naphthaldehyde (2.00 g, 12.8 mmol, 1.74 mL, 1.0 eq). The mixture was stirred at 20° C. for 48 h. LCMS indicated that 53% of the starting material remained and 30% of the desired mass was detected. The reaction mixture was poured into H₂O (25 mL) and extracted with EtOAc (8.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/3. (F)-2-Methyl-N-(naphthalen-1-ylmethylene) propane-2-sulfinamide (2.00 g, 3.01 mmol, 23% yield) was obtained as a yellow oil. M+H⁺=260.2 (LCMS); ¹H NMR (400 MHZ, CDCl₃) δ 9.17 (s, 1H), 9.07-9.02 (m, 1H), 8.08-8.01 (m, 2H), 7.96-7.92 (m, 1H), 7.70-7.63 (m, 1H), 7.62-7.54 (m, 2H), 1.34 (s, 9H).

### Step 2: N-(2,2-Difluoro-1-(naphthalen-1-yl)ethyl)-2-methylpropane-2-sulfinamide (38A-2)

To a solution of (E)-2-methyl-N-(naphthalen-1-ylmethylene) propane-2-sulfinamide (500 mg, 1.93 mmol, 1.0 eq) and (difluoromethyl)trimethylsilane (718 mg, 5.78 mmol, 3.0 eq) in THF (6.0 mL) was added 1-BuOK (1 M in THF, 5.78 mL, 3.0 eq). The mixture was degassed and purged with N₂ three times, stirred at −70° C. for 10 min, then stirred at 20° C. for another 30 min under a N₂ atmosphere. LCMS indicated that the starting material was completely consumed, and the desired mass was detected. The reaction mixture was poured into H₂O (6.0 mL) and extracted with EtOAc (2.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/3. N-(2,2-Difluoro-1-(naphthalen-1-yl)ethyl)-2-methylpropane-2-sulfinamide (270 mg, 819 μmol, 43% yield) was obtained as a brown oil. M+H⁺=312.1 (LCMS).

### Step 3: 2,2-Difluoro-1-(naphthalen-1-yl)ethanamine hydrochloride (38A-3)

To a solution of N-(2,2-difluoro-1-(naphthalen-1-yl)ethyl)-2-methylpropane-2-sulfinamide (100 mg, 321 μmol, 1.0 eq) in EtOAc (1.0 mL) was added HCl/EtOAc (4 M, 4.0 mL). The mixture was stirred at 20° C. for 20 min. LCMS indicated that the starting material was completely consumed, and the desired mass was detected. The mixture was concentrated under vacuum to give the crude product 2,2-difluoro-1-(naphthalen-1-yl)ethanamine hydrochloride (100 mg) as a white solid, which was used in the next step without any further purification. M+H⁺=208.2 (LCMS).

### Step 4: N-(2,2-Difluoro-1-(naphthalen-1-yl)ethyl)-2-methyl-5-nitrobenzamide (38A-4)

To a solution of 2,2-difluoro-1-(naphthalen-1-yl)ethanamine (100 mg, 410 μmol, 1.0 eq, HCl salt) and 2-methyl-5-nitrobenzoic acid (81.8 mg, 451 μmol, 1.1 eq) in DCM (5.0 mL) were added TEA (125 mg, 1.23 mmol, 171 μL, 3.0 eq), EDCI (157 mg, 821 μmol, 2.0 eq) and HOBt (111 mg, 821 μmol, 2.0 eq). The mixture was stirred at 20° C. for 16 h. LCMS indicated that the starting material was completely consumed, and the desired mass was detected. The reaction mixture was poured into H₂O (5.0 mL) and extracted with EtOAc (3.0 mL×2). The combined organic layers were dried over Na₂SO₄, filtered, and concentrated under vacuum to give a residue which was purified by flash silica gel chromatography using a gradient of EtOAc/petroleum ether from 1/100 to 1/3. N-(2,2-Difluoro-1-(naphthalen-1-yl)ethyl)-2-methyl-5-nitrobenzamide (60.0 mg, 1.62 mmol, 39% yield) was obtained as a white solid. M+H⁺=371.2 (LCMS).

### Step 5: 5-Amino-N-(2,2-difluoro-1-(naphthalen-1-yl)ethyl)-2-methylbenzamide (Compound 185)

To a solution of N-(2,2-difluoro-1-(naphthalen-1-yl)ethyl)-2-methyl-5-nitrobenzamide (50.0 mg, 135 μmol, 1.0 eq) in MeOH (5.0 mL) were added H₂O (1.0 mL), iron powder (37.7 mg, 675 μmol, 5.0 eq) and NH₄Cl (36.1 mg, 675 μmol, 5.0 eq). The mixture was stirred at 80° C. for 2 h. LCMS indicated that the starting material was completely consumed, and the desired mass was detected. The reaction mixture was allowed to cool to room temperature, poured into H₂O (6.0 mL) and extracted with EtOAc (3.0 mL×2). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 5%-40% B over 8 min; mobile phase A: 0.04% aqueous HCl, mobile phase B: acetonitrile). 5-Amino-N-(2,2-difluoro-1-(naphthalen-1-yl)ethyl)-2-methylbenzamide was obtained as a white solid. M+H⁺=341.0 (LCMS); ¹H NMR (400 MHZ, DMSO-d₆) δ 10.13-9.65 (m, 1H), 9.49 (br d, J=8.8 Hz, 1H), 8.31-8.23 (m, 1H), 8.04-7.94 (m, 2H), 7.80-7.74 (m, 1H), 7.69-7.53 (m, 3H), 7.34-7.19 (m, 2H), 7.15 (s, 1H), 6.71-6.66 (m, 1H), 6.59-6.52 (m, 1H), 2.29-2.19 (m, 3H).

### Example 39: 5-Amino-2-methyl-N-(2,2,2-trifluoro-1-(naphthalen-1-yl)ethyl)benzamide (Compound 183)

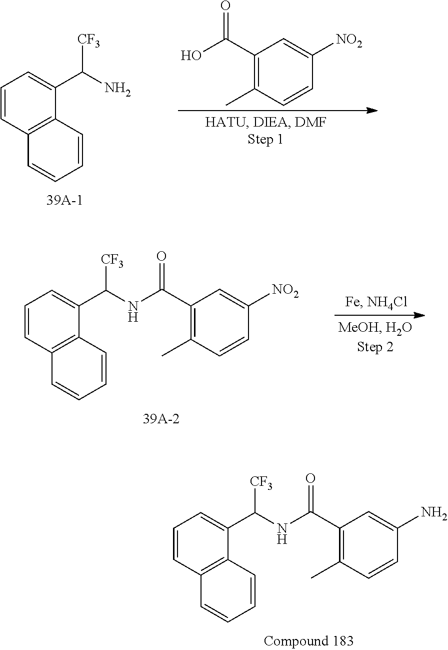 [SMILES: NC(c1cccc2ccccc12)C(F)(F)F ; Cc1ccc([N+](=O)[O-])cc1C(=O)O ; Cc1ccc([N+](=O)[O-])cc1C(=O)NC(c1cccc2ccccc12)C(F)(F)F ; Cc1ccc(N)cc1C(=O)NC(c1cccc2ccccc12)C(F)(F)F]

### Step 1: 2-Methyl-5-nitro-N-(2,2,2-trifluoro-1-(naphthalen-1-yl)ethyl)benzamide (39A-2)

To a solution of 2-methyl-5-nitrobenzoic acid (88.5 mg, 488 μmol, 1.1 eq), 2,2,2-trifluoro-1-(naphthalen-1-yl)ethanamine (100 mg, 444 μmol, 1.0 eq) in DMF (10 mL) was added DIEA (172 mg, 1.33 mmol, 232 μL, 3.0 eq). The mixture was stirred at 25° C. for 5 min then HATU (186 mg, 488 μmol, 1.1 eq) was added. The resulting mixture was stirred at 25° C. for 16 h. LCMS indicated that the starting material was completely consumed, and the desired mass was detected. The reaction mixture was poured into H₂O (20 mL), and a precipitate was formed. The mixture was filtered, and the filter cake was washed with H₂O (5.0 mL) and dried under vacuum to give 2-methyl-5-nitro-N-(2,2,2-trifluoro-1-(naphthalen-1-yl)ethyl)benzamide (120 mg, 309 μmol, 70% yield) as a white solid. M+H⁺=389.1 (LCMS); ¹H NMR (400 MHZ, CDCl₃) δ 8.27-8.15 (m, 3H), 7.96 (t, J=6.9 Hz, 2H), 7.75-7.65 (m, 2H), 7.63-7.51 (m, 2H), 7.42 (d, J=8.1 Hz, 1H), 6.89-6.78 (m, 1H), 6.44 (br d, J=9.3 Hz, 1H), 2.54 (s, 3H).

### Step 2: 5-Amino-2-methyl-N-(2,2,2-trifluoro-1-(naphthalen-1-yl)ethyl)benzamide (Compound 183)

To a stirred solution of 2-methyl-5-nitro-N-(2,2,2-trifluoro-1-(naphthalen-1-yl)ethyl)benzamide (110 mg, 283 μmol, 1.0 eq) in a mixture of MeOH (10 mL) and H₂O (2.5 mL) was added iron powder (79.0 mg, 1.42 mmol, 5.0 eq), followed by NH₄Cl (75.8 mg, 1.42 mmol, 5.0 eq). The mixture was stirred at 80° C. for 16 h. LCMS indicated that the starting material was completely consumed, and the desired product was detected. The reaction mixture was allowed to cool to room temperature. The suspension was filtered through a pad of Celite and the combined filtrates were poured into H₂O (10 mL) and extracted with EtOAc (10 mL×4). The combined organic layers were dried over Na₂SO₄, filtered, and concentrated under vacuum to give a residue which was purified by preparative HPLC (Waters Xbridge BEH C18 (100×30 mm, 3 μm); flow rate: 25 mL/min; gradient: 50%-70% B over 8 min; mobile phase A: 10 mM aqueous NH₄HCO₃, mobile phase B: acetonitrile) to give 5-amino-2-methyl-N-(2,2,2-trifluoro-1-(naphthalen-1-yl)ethyl)benzamide (39.7 mg, 110 μmol, 39% yield) as a white solid. M+H⁺=359.0 (LCMS); ¹H NMR (400 MHZ, CDCl₃) δ 8.24 (d, J=8.6 Hz, 1H), 7.95-7.89 (m, 2H), 7.65 (dt, J=1.4, 8.6 Hz, 2H), 7.61-7.55 (m, 1H), 7.54-7.47 (m, 1H), 6.99 (d, J=8.9 Hz, 1H), 6.88-6.73 (m, 1H), 6.69-6.58 (m, 2H), 6.36 (br d, J=9.5 Hz, 1H), 3.60 (br s, 2H), 2.29 (s, 3H).

### Example 40: 5-Amino-2-methyl-N-(3-(naphthalen-1-yl)oxetan-3-yl)benzamide (Compound 191)

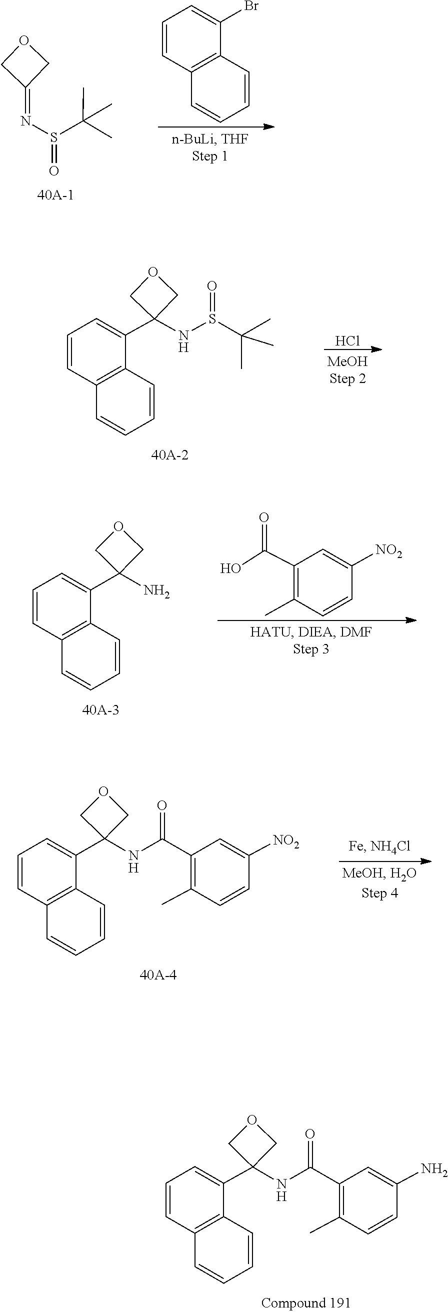

### Step 1: 2-Methyl-N-(3-(naphthalen-1-yl)oxetan-3-yl)propane-2-sulfinamide (40A-2)

To a solution of 1-bromonaphthalene (886 mg, 4.28 mmol, 595 μL, 1.5 eq) in THF (10 mL) was added n-BuLi (2.5 M in hexane, 1.60 mL, 1.4 eq) dropwise at −78° C. under a N₂ atmosphere. The resulting mixture was stirred at −78° C. for 1 h, then a solution of 2-methyl-N-(oxetan-3-ylidene) propane-2-sulfinamide (500 mg, 2.85 mmol, 1.0 eq) in THF (5.0 mL) was added dropwise at −78° C. The resulting mixture was stirred at −78° C. for 1 h. TLC indicated that that the starting material was completely consumed. The reaction mixture was poured into saturated aqueous NH₄Cl (20 mL) and extracted with EtOAc (10 mL×4). The combined organic layers were dried over Na₂SO₄, filtered, and concentrated under vacuum to give a residue which was purified by flash silica gel chromatography using a gradient of EtOAc/petroleum ether from 0/1 to 1/0. 2-Methyl-N-(3-(naphthalen-1-yl)oxetan-3-yl)propane-2-sulfinamide (450 mg, 1.48 mmol, 52% yield) was obtained as a yellow gum.

### Step 2: 3-(Naphthalen-1-yl)oxetan-3-amine (40A-3)

To a solution of 2-methyl-N-(3-(naphthalen-1-yl)oxetan-3-yl)propane-2-sulfinamide (250 mg, 824 μmol, 1.0 eq) in MeOH (3.0 mL) was added HCl/dioxane (6 M, 1.0 mL) at 0° C. The resulting mixture was stirred at 0° C. for 10 min. LCMS indicated that the starting material was completely consumed, and the desired product was detected. The reaction mixture was concentrated under vacuum to give 3-(1-naphthyl)oxetan-3-amine (190 mg, 806 μmol, 98% yield, HCl salt) as a white solid, which was used in the next step without any further purification.

### Step 3: 2-Methyl-N-(3-(naphthalen-1-yl)oxetan-3-yl)-5-nitrobenzamide (40A-4)

To a solution of 2-methyl-5-nitrobenzoic acid (84.5 mg, 467 μmol, 1.1 eq) and 3-(naphthalen-1-yl)oxetan-3-amine (100 mg, 424 μmol, 1.0 eq, HCl salt) in DMF (10 mL) was added DIEA (164 mg, 1.27 mmol, 222 μL, 3.0 eq). After stirring for 5 min, HATU (177 mg, 467 μmol, 1.1 eq) was added. The resulting mixture was stirred at 25° C. for 3 h. TLC indicated that that the starting material was completely consumed. The reaction mixture was poured into H₂O (20 mL), and a precipitate was formed. The mixture was filtered and the solid was washed H₂O (5.0 mL) and dried under vacuum to give 2-methyl-N-(3-(naphthalen-1-yl)oxetan-3-yl)-5-nitrobenzamide (150 mg, 414 μmol, 98% yield) as a white solid. ¹H NMR (400 MHZ, CDCl₃) δ8.12 (br d, J=2.4 Hz, 2H), 8.01-7.93 (m, 1H), 7.89 (d, J=8.3 Hz, 1H), 7.77 (d, J=7.3 Hz, 1H), 7.56 (s, 3H), 7.42 (br d, J=8.8 Hz, 1H), 7.33 (br d, J=9.1 Hz, 1H), 6.65 (br s, 1H), 5.61-5.42 (m, 4H), 2.37 (s, 3H).

### Step 4: 5-Amino-2-methyl-N-(3-(naphthalen-1-yl)oxetan-3-yl)benzamide (Compound 191)

To a stirred solution of 2-methyl-N-(3-(naphthalen-1-yl)oxetan-3-yl)-5-nitrobenzamide (80.0 mg, 221 μmol, 1.0 eq) in a mixture of MeOH (8.0 mL) and H₂O (2.0 mL) was added iron powder (61.7 mg, 1.10 mmol, 5.0 eq), followed by NH₄Cl (59.0 mg, 1.10 mmol, 5.0 eq). The mixture was stirred at 80° C. for 16 h. LCMS indicated that the starting material was completely consumed, and the desired mass was detected. The reaction mixture was allowed to cool to room temperature. The suspension was filtered through a pad of Celite and filtrate was poured into H₂O (10 mL) and extracted with EtOAc (10 mL×4). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 20%-50% B over 8 min; mobile phase A: 10 mM aqueous NH₄HCO₃, mobile phase B: acetonitrile) to give 5-amino-2-methyl-N-(3-(naphthalen-1-yl)oxetan-3-yl)benzamide (19.1 mg, 56.6 μmol, 26% yield) as a white solid. M+H⁺=333.0 (LCMS); ¹H NMR (400 MHZ, CDCl₃) δ 7.94 (dd, J=3.6, 5.9 Hz, 1H), 7.85 (d, J=8.1 Hz, 1H), 7.76 (d, J=7.1 Hz, 1H), 7.59-7.46 (m, 3H), 7.43-7.36 (m, 1H), 6.91 (d, J=8.0 Hz, 1H), 6.69-6.53 (m, 3H), 5.50 (d, J=6.6 Hz, 2H), 5.46-5.39 (m, 2H), 2.14 (s, 3H).

### Example 41: 5-(2-Aminoethoxy)-2-methyl-N-(1-(naphthalen-1-yl)cyclopropyl)benzamide (Compound 281)

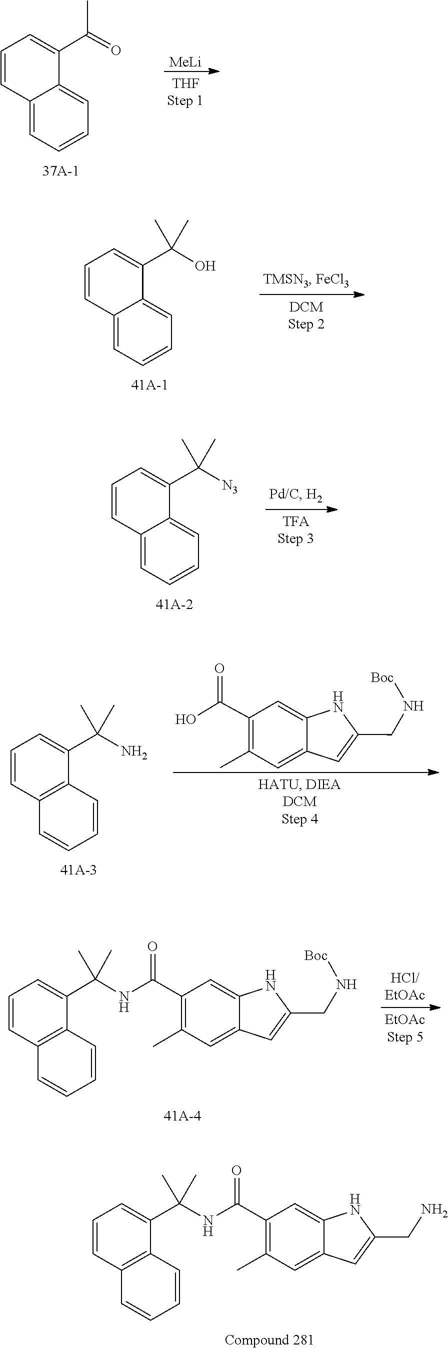

### Step 1: 2-(Naphthalen-1-yl)propan-2-ol (41A-1)

A mixture of 1-(naphthalen-1-yl)ethanone (10.0 g, 58.8 mmol, 1.0 eq) in THF (100 mL) was degassed and purged with N₂ three times. To this mixture was added methyl lithium (1.6 M in Et₂O, 73.4 mL, 2.0 eq) dropwise to maintain the temperature between −78° C. and −75° C. under a N₂ atmosphere. After the addition was complete, the resulting mixture was stirred at the same temperature for 2 h and warmed to 25° C. stirring for another 1 h. TLC indicated that the starting material was completely consumed. The reaction mixture was poured into H₂O (50 mL) and extracted with EtOAc (50 mL×3). The combined organic layers were dried over Na₂SO₄, filtered, and concentrated under vacuum to give a residue which was purified by flash silica gel chromatography using a gradient of EtOAc/petroleum ether from 0/1 to 1/9. 2-(Naphthalen-1-yl)propan-2-ol (4.00 g, 21.3 mmol, 36% yield) was obtained as a white solid. M−18+H⁺=169.1 (LCMS).

### Step 2: 1-(2-Azidopropan-2-yl) naphthalene (41A-2)

A mixture of 2-(naphthalen-1-yl)propan-2-ol (1.00 g, 5.37 mmol, 1.0 eq), FeCl₃ (87.1 mg, 537 μmol, 0.1 eq) in DCM (10 mL) was degassed and purged with N₂ three times. To the mixture was added TMSN₃ (1.24 g, 10.7 mmol, 1.41 mL, 2.0 eq) dropwise at 0° C. The resulting mixture was stirred at 25° C. for 2 h under a N₂ atmosphere. TLC indicated that the starting material was completely consumed. The reaction mixture was filtered to give a residue which was purified by column chromatography using a gradient of DCM. 1-(2-Azidopropan-2-yl) naphthalene (1.00 g, 4.73 mmol, 88% yield) was obtained as a colorless oil.

### Step 3: 2-(Naphthalen-1-yl)propan-2-amine (41A-3)

To a solution of 1-(2-azidopropan-2-yl) naphthalene (1.00 g, 4.73 mmol, 1.0 eq) in trifluoroethanol (2.0 mL) was added 10% palladium on carbon (100 mg) at 25° C. in one portion. After degassing and purging with H₂ three times, the mixture was stirred at 25° C. for 1 h under a H₂ (15 psi) atmosphere. LCMS indicated that the starting material was completely consumed, and the desired mass was detected. The reaction mixture was filtered and the filtrated was concentrated under vacuum to give the crude product 2-(naphthalen-1-yl)propan-2-amine (500 mg, 2.70 mmol, 63% yield), which was used in the next step without any further purification. M−17+H⁺=169.1 (LCMS).

### Step 4: tert-Butyl((5-methyl-6-((2-(naphthalen-1-yl)propan-2-yl)carbamoyl)-1H-indol-2-yl)methyl)carbamate (41A-4)

To a solution of 2-(naphthalen-1-yl)propan-2-amine (100 mg, 540 μmol, 1.0 eq) and 2-(((tert-butoxycarbonyl)amino)methyl)-5-methyl-1H-indole-6-carboxylic acid (197 mg, 648 μmol, 1.2 eq) in DCM (3.0 mL) were added HATU (308 mg, 810 μmol, 1.5 eq), DIEA (209 mg, 1.62 mmol, 3.0 eq). The mixture was stirred at 25° C. for 16 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by preparative TLC (EtOAc/petroleum ether=1:1, R_{f}=0.3). tert-Butyl((5-methyl-6-((2-(naphthalen-1-yl)propan-2-yl)carbamoyl)-1H-indol-2-yl)methyl)carbamate (100 mg, 212 μmol, 39% yield) was obtained as a yellow oil. M+H⁺=472.2 (LCMS).

### Step 5: 5-(2-Aminoethoxy)-2-methyl-N-(1-(naphthalen-1-yl)cyclopropyl)benzamide (Compound 281)

To a stirred solution of tert-butyl((5-methyl-6-((2-(naphthalen-1-yl)propan-2-yl)carbamoyl)-1H-indol-2-yl)methyl)carbamate (100 mg, 212 μmol, 1.0 eq) in EtOAc (1.0 mL) was added HCl/EtOAc (4 M, 3.0 mL). The mixture was stirred at 25° C. for 1 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 5%-50% B over 8 min; mobile phase A: 0.04% aqueous HCl, mobile phase B: acetonitrile). 5-(2-Aminoethoxy)-2-methyl-N-(1-(naphthalen-1-yl)cyclopropyl)benzamide (45.3 mg, 119 μmol, 56% yield, HCl salt) was obtained as a white solid. M+H⁺=372.1 (LCMS); ¹H NMR (400 MHZ, DMSO-d₆) δ 11.25-11.07 (m, 1H), 8.83-8.75 (m, 2H), 8.49-8.23 (m, 3H), 7.94 (dd, J=3.1, 6.4 Hz, 1H), 7.80 (d, J=8.5 Hz, 1H), 7.61 (d, J=7.1 Hz, 1H), 7.52-7.45 (m, 3H), 7.29 (d, J=5.0 Hz, 2H), 6.43 (s, 1H), 4.17 (br d, J=5.8 Hz, 2H), 2.14 (s, 3H), 1.89 (s, 6H).

### Example 42: 2-(Aminomethyl)-5-methyl-N-(naphthalen-1-ylmethyl)-1H-indole-6-carboxamide (Compound 241)

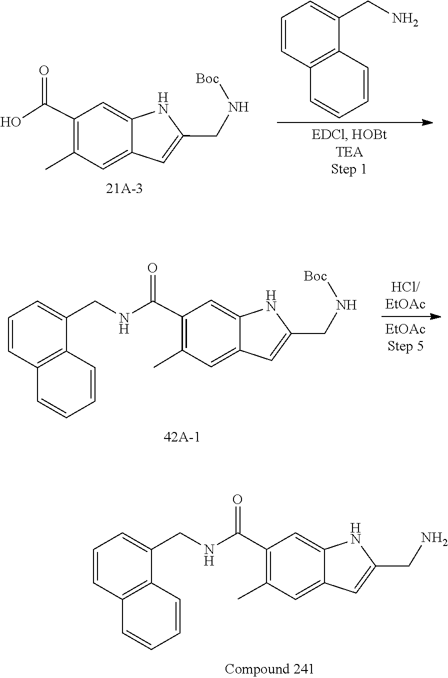 [SMILES: Cc1cc2cc(CNC(=O)OC(C)(C)C)[nH]c2cc1C(=O)O ; NCc1cccc2ccccc12 ; Cc1cc2cc(CNC(=O)OC(C)(C)C)[nH]c2cc1C(=O)NCc1cccc2ccccc12 ; Cc1cc2cc(CN)[nH]c2cc1C(=O)NCc1cccc2ccccc12]

### Step 1: tert-Butyl((5-methyl-6-((naphthalen-1-ylmethyl)carbamoyl)-1H-indol-2-yl)methyl)carbamate (42A-1)

To a solution of 2-(((tert-butoxycarbonyl)amino)methyl)-5-methyl-1H-indole-6-carboxylic acid (120 mg, 394 μmol, 1.0 eq) and 5 naphthalen-1-ylmethanamine (62.0 mg, 394 μmol, 1.0 eq) in DCM (4.0 mL) were added TEA (120 mg, 1.18 mmol, 165 μL, 3.0 eq), EDCI (189 mg, 986 μmol, 2.5 eq) and HOBt (133 mg, 986 μmol, 2.5 eq). The mixture was stirred at 20° C. for 16 h. LCMS indicated that the starting material was completely consumed, and the desired mass was detected. The reaction mixture was poured into H₂O (10 mL) and extracted with EtOAc (10 mL×2). The combined organic layers were dried over Na₂SO₄, filtered, and concentrated under vacuum to give a residue which was purified by flash silica gel chromatography using a gradient of EtOAc/petroleum ether from 0/100 to 1/1. tert-Butyl((5-methyl-6-((naphthalen-1-ylmethyl)carbamoyl)-1H-indol-2-yl)methyl)carbamate (60.0 mg, 73.1 μmol, 19% yield) was obtained as a yellow solid. M+H⁺=444.2 (LCMS).

### Step 2: 2-(Aminomethyl)-5-methyl-N-(naphthalen-1-ylmethyl)-1H-indole-6-carboxamide (Compound 241)

To a stirred solution of tert-butyl((5-methyl-6-((naphthalen-1-ylmethyl)carbamoyl)-1H-indol-2-yl)methyl)carbamate (60.0 mg, 73.1 μmol, 1.0 eq) in EtOAc (1.0 mL) was added HCl/EtOAc (4 M, 5.0 mL). The mixture was stirred at 25° C. for 1 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 10%-35% B over 8 min; mobile phase A: 0.04% aqueous HCl, mobile phase B: acetonitrile). 2-(Aminomethyl)-5-methyl-N-(naphthalen-1-ylmethyl)-1H-indole-6-carboxamide (18.9 mg, 54.4 μmol, 39% yield, HCl salt) was obtained as a white solid. M+H⁺=344.1 (LCMS); 1H NMR (400 MHZ, DMSO-d₆) δ 11.38 (s, 1H), 8.77 (t, J=5.8 Hz, 1H), 8.55 (br s, 3H), 8.24 (d, J=7.9 Hz, 1H), 8.01-7.94 (m, 1H), 7.87 (d, J=7.6 Hz, 1H), 7.62-7.47 (m, 4H), 7.43 (s, 1H), 7.35 (s, 1H), 6.47 (s, 1H), 4.93 (d, J=5.8 Hz, 2H), 4.17 (q, J=5.7 Hz, 2H), 2.41 (s, 3H).

### Example 43: 5-Amino-2-methyl-N-(1-(naphthalen-1-yl)cyclopropyl)benzamide (Compound 147)

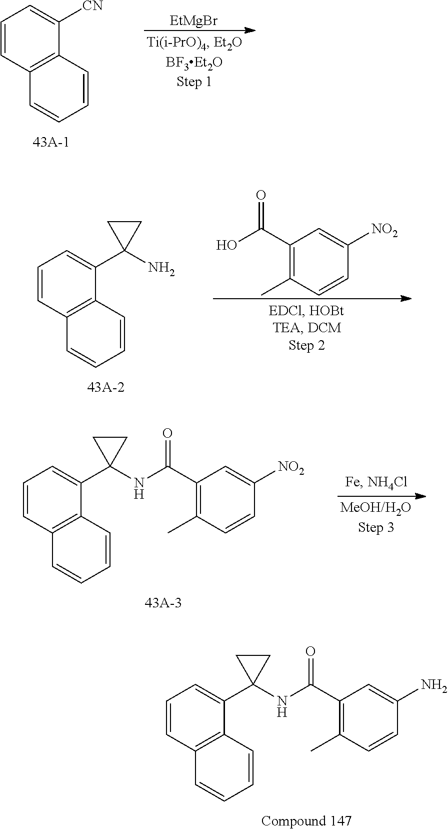 [SMILES: "EtMgBr" ; N#Cc1cccc2ccccc12 ; NC1(c2cccc3ccccc23)CC1 ; Cc1ccc([N+](=O)[O-])cc1C(=O)O ; Cc1ccc([N+](=O)[O-])cc1C(=O)NC1(c2cccc3ccccc23)CC1 ; Cc1ccc(N)cc1C(=O)NC1(c2cccc3ccccc23)CC1]

### Step 1: 1-(Naphthalen-1-yl)cyclopropanamine (43A-2)

A mixture of 1-naphthonitrile (30.0 g, 196 mmol, 1.0 eq) in anhydrous Et₂O (1000 mL) was degassed and purged with N₂ three times. The mixture was stirred with a mechanical stirrer at −78° C. To this mixture was added Ti(i-PrO)₄ (61.2 g, 215 mmol, 63.4 mL, 1.1 eq) slowly, and then EtMgBr (3 M in Et₂O, 144 mL, 2.2 eq) was added dropwise to maintain the temperature between −78° C. and −75° C. over 1 h under a N₂ atmosphere. After the addition was complete, the resulting mixture was stirred at the same temperature for 10 min and warmed to 20° C. over 1 h. BF₃·Et₂O (55.6 g, 392 mmol, 48.3 mL, 2.0 eq) was added slowly with no obvious temperature change. The mixture was stirred at 20° C. for another 1 h. TLC indicated that the starting material was completely consumed, and a main spot was detected. The reaction mixture was added into a mixture of HCl (1 M aqueous) (500 mL) and MTBE (500 mL), and extracted with MTBE (500 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (500 mL×2). The combined organic layers were dried over Na₂SO₄, filtered, and concentrated under vacuum to give a residue which was purified by flash silica gel chromatography using a gradient of EtOAc/petroleum ether from 0/1 to 1/1. 1-(Naphthalen-1-yl)cyclopropanamine (15.0 g, 81.9 mmol, 21% yield) was obtained as a yellow oil. M+H⁺=184.2 (LCMS); ¹H NMR (400 MHZ, CDCl₃) δ 8.47-8.45 (br d, J=8.31 Hz, 1H), 7.91 (br d, J=8.19 Hz, 1H), 7.78 (br d, J=8.19 Hz, 1H), 7.67-7.57 (m, 1H), 7.56-7.50 (m, 2H), 7.46-7.38 (m, 1H), 1.25-1.18 (m, 2H), 1.09-1.00 (m, 2H).

### Step 2: 2-Methyl-N-(1-(naphthalen-1-yl)cyclopropyl)-5-nitrobenzamide (43A-3)

To a solution of 1-(naphthalen-1-yl)cyclopropanamine (70.0 mg, 382 μmol, 1.0 eq) and 2-methyl-5-nitrobenzoic acid (76.1 mg, 420 μmol, 1.1 eq) in DCM (2.0 mL) were added TEA (116 mg, 1.15 mmol, 160 μL, 3.0 eq), EDCI (87.9 mg, 458 μmol, 1.2 eq) and HOBt (61.9 mg, 458 μmol, 1.2 eq). The mixture was stirred at 20° C. for 3 h. LCMS indicated that starting material was completely consumed, and the desired mass was detected. The reaction mixture was poured into H₂O (5.0 mL) and extracted with DCM (3.0 mL×3). The combined organic layers were dried over Na₂SO₄, filtered, and concentrated under vacuum to give a residue which was purified by preparative TLC (EtOAc/petroleum ether=1/2, R_{f}=0.7). 2-Methyl-N-(1-(naphthalen-1-yl)cyclopropyl)-5-nitrobenzamide (50.0 mg, 144 μmol, 38% yield) was obtained as a white solid. M+H⁺=347.2 (LCMS).

### Step 3: 5-Amino-2-methyl-N-(1-(naphthalen-1-yl)cyclopropyl)benzamide (Compound 147)

To a solution of 2-methyl-N-(1-(naphthalen-1-yl)cyclopropyl)-5-nitrobenzamide (30.0 mg, 86.7 μmol, 1.0 eq) in a mixture of MeOH (2.0 mL) and H₂O (0.5 mL) were added iron powder (24.2 mg, 433 μmol, 5.0 eq) and NH₄Cl (23.2 mg, 433 μmol, 5.0 eq). The resulting mixture was stirred at 80° C. for 2 h. LCMS indicated that the starting material was completely consumed, and the desired mass was detected. The reaction mixture was allowed to cool to room temperature, poured into H₂O (5.0 mL) and extracted with EtOAc (3.0 mL×3). The combined organic layers were dried over Na₂SO₄, filtered, and concentrated under vacuum to give a residue which was purified by preparative HPLC (Phenomenex Luna C18 column (80× 30 mm, 3 μm); flow rate: 25 mL/min; gradient: 5%-50% B over 8 min; mobile phase A: 0.04% aqueous HCl, mobile phase B: acetonitrile). 5-Amino-2-methyl-N-(1-(naphthalen-1-yl)cyclopropyl)benzamide (10.1 mg, 28.4 μmol, 33% yield, HCl salt) was obtained as a white solid. M+H⁺=317.0 (LCMS); ¹H NMR (400 MHZ, CD₃OD) δ 9.33 (s, 1H), 8.56 (d, J=8.3 Hz, 1H), 7.90 (d, J=7.5 Hz, 2H), 7.82 (d, J=8.3 Hz, 1H), 7.60-7.42 (m, 3H), 7.30-7.26 (m, 2H), 7.03 (d, J=2.0 Hz, 1H), 2.20-1.92 (m, 3H), 1.55-1.39 (m, 2H), 1.38-1.25 (m, 2H).

### Example 44: 5-Amino-2-methyl-N-(1-(naphthalen-1-yl)cyclobutyl)benzamide (Compound 219)

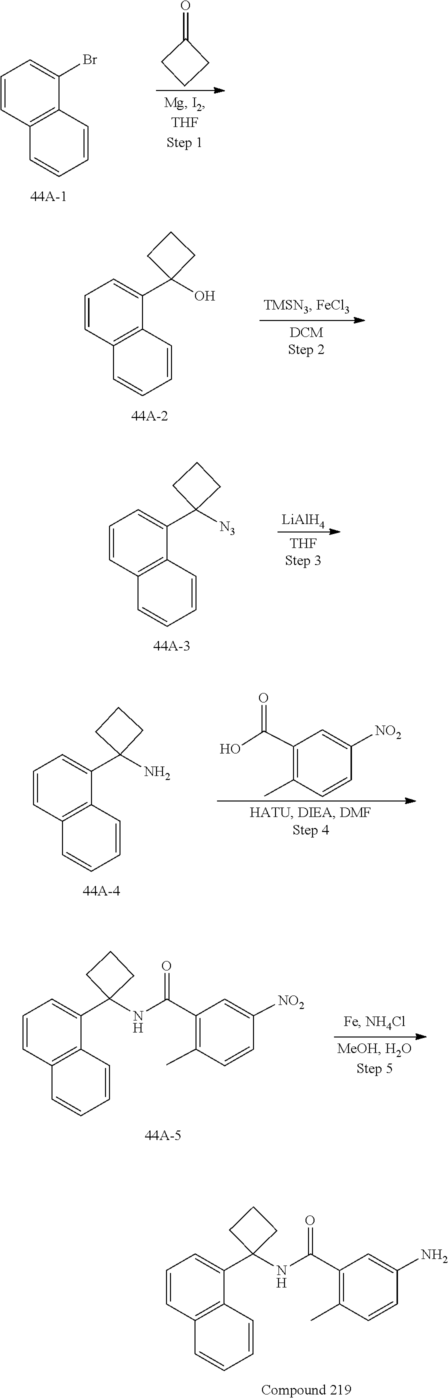

### Step 1: 1-(Naphthalen-1-yl)cyclobutanol (44A-2)

A test tube with magnesium (572 mg, 23.5 mmol, 1.65 eq) was flame dried under vacuum. After cooling to 25° C. under argon, I₂ (36.2 mg, 143 μmol, 0.01 eq) and THF (21 mL) were added, followed by a solution of 1-bromonaphthalene (4.43 g, 21.4 mmol, 10.7 mL, 1.5 eq) in THF (10 mL) in portions at 25° C. The reaction mixture was then stirred at 70° C. for 1.5 h. The mixture was cooled to 0° C. and cyclobutanone (1.00 g, 14.3 mmol, 1.10 mL, 1.0 eq) was added dropwise. The resulting mixture was stirred for 16 h at 25° C. LCMS indicated that the starting material was completely consumed. The reaction mixture was poured into saturated aqueous NH₄Cl (50 mL) and extracted with EtOAc (20 mL×4). The combined organic layers were dried over Na₂SO₄, filtered, and concentrated under vacuum to give a residue which was purified by flash silica gel chromatography using a gradient of EtOAc/petroleum ether from 0/1 to 1/1. 1-(Naphthalen-1-yl)cyclobutanol (2.40 g, 12.1 mmol, 8% yield) was obtained as a white solid. ¹H NMR (400 MHZ, CDCl₃) δ 8.36-8.25 (m, 1H), 7.93-7.86 (m, 1H), 7.81 (d, J=8.2 Hz, 1H), 7.57-7.48 (m, 3H), 7.47-7.41 (m, 1H), 2.88 (ddd, J=5.9, 8.8, 12.3 Hz, 2H), 2.64 (ddd, J=6.7, 9.3, 12.3 Hz, 2H), 2.32-2.22 (m, 1H), 2.17 (dddd, J=3.4, 5.6, 9.2, 11.2 Hz, 1H), 1.79-1.64 (m, 1H).

### Step 2: 1-(1-Azidocyclobutyl) naphthalene (44A-3)

To a solution of 1-(naphthalen-1-yl)cyclobutanol (500 mg, 2.52 mmol, 1.0 eq) and azidotrimethylsilane (349 mg, 3.03 mmol, 1.2 eq) in DCM (13 mL) was added FeCl₃ (8.18 mg, 50.4 μmol, 0.02 eq) at 0° C. under a N₂ atmosphere. Then the reaction mixture was stirred at 25° C. for 2 h. LCMS indicated that the starting material was completely consumed. The reaction mixture was quenched by H₂O (30 mL) and extracted with DCM (10 mL×4). The combined organic layers were dried over Na₂SO₄, filtered, and concentrated under vacuum to give crude product 1-(1-azidocyclobutyl) naphthalene (560 mg) as a yellow gum, which was used in the next step without any further purification.

### Step 3: 1-(Naphthalen-1-yl)cyclobutanamine (44A-4)

To a solution of 1-(1-azidocyclobutyl) naphthalene (100 mg, 448 μmol, 1.0 eq) in THF (4.0 mL) was added LiAlH₄ (17.9 mg, 470 μmol, 1.0 eq) at 0° C. under a N₂ atmosphere. Then the reaction mixture was stirred at 25° C. for 3 h. LCMS indicated that the starting material was completely consumed, and the desired mass was detected. The reaction mixture was quenched by the addition of saturated aqueous NH₄Cl (10 mL) and extracted with DCM (10 mL×4). The combined organic layers were dried over Na₂SO₄, filtered, and concentrated under vacuum to give crude product 1-(naphthalen-1-yl)cyclobutanamine as a yellow gum, which was used in the next step without any further purification. M+H⁺=198.1 (LCMS).

### Step 4: 2-Methyl-N-(1-(naphthalen-1-yl)cyclobutyl)-5-nitrobenzamide (44A-5)

To a solution of 2-methyl-5-nitrobenzoic acid (70.7 mg, 390 μmol, 1.1 eq) and 1-(naphthalen-1-yl)cyclobutanamine (70.0 mg, 355 μmol, 1.0 eq) in DMF (4 mL) was added DIEA (138 mg, 1.06 mmol, 185 μL, 3.0 eq). After stirring 5 min, HATU (148 mg, 390 μmol, 1.1 eq) was added. The resulting mixture was stirred at 25° C. for 16 h. TLC indicated that the starting material was completely consumed. The reaction mixture was poured into H₂O (10 mL), and then extracted with EtOAc (10 mL×3). The combined organic layers were washed with brine (10 mL), dried over Na₂SO₄, filtered, and concentrated under vacuum to give a residue which was purified by preparative TLC (EtOAc/petroleum ether=1/3, R_{f}=0.4). 2-Methyl-N-(1-(naphthalen-1-yl)cyclobutyl)-5-nitrobenzamide (100 mg, 277 μmol, 78% yield) was obtained as a white solid.

### Step 5: 5-Amino-2-methyl-N-(1-(naphthalen-1-yl)cyclobutyl)benzamide (Compound 219)

To a stirred solution of 2-methyl-N-(1-(naphthalen-1-yl)cyclobutyl)-5-nitrobenzamide (100 mg, 277 μmol, 1.0 eq) in a mixture of MeOH (10 mL) and H₂O (2.5 mL) was added iron powder (77.5 mg, 1.39 mmol, 5.0 eq), followed by NH₄Cl (74.2 mg, 1.39 mmol, 5.0 eq). The mixture was stirred at 80° C. for 16 h. LCMS indicated that the starting material was completely consumed, and the desired mass was detected. The reaction mixture was allowed to cool to room temperature. The suspension was filtered through a pad of Celite and the filtrate were poured into H₂O (10 mL) and extracted with EtOAc (10 mL×4). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 35%-65% B over 10 min; mobile phase A: 10 mM aqueous NH₄HCO₃, mobile phase B: acetonitrile). 5-Amino-2-methyl-N-(1-(naphthalen-1-yl)cyclobutyl)benzamide (35.9 mg, 107 μmol, 39% yield) was obtained as a white solid. M+H⁺=331.1 (LCMS); ¹H NMR (400 MHZ, CDCl₃) δ 8.03-7.96 (m, 1H), 7.95-7.89 (m, 1H), 7.80 (d, J=7.4 Hz, 2H), 7.56-7.42 (m, 3H), 6.90 (d, J=8.0 Hz, 1H), 6.62-6.50 (m, 2H), 6.27 (br s, 1H), 3.67-3.38 (m, 2H), 3.25 (ddd, J=6.1, 9.3, 12.6 Hz, 2H), 3.03-2.88 (m, 2H), 2.37-2.26 (m, 1H), 2.19 (s, 3H), 1.95-1.82 (m, 1H).

### Example 45: 5-(Azetidin-3-ylamino)-2-methyl-N-(1-(naphthalen-1-yl)cyclopropyl)benzamide (Compound 177)

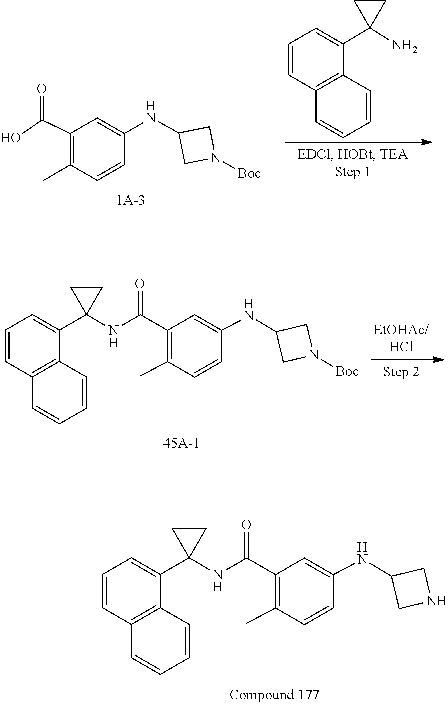 [SMILES: Cc1ccc(NC2CN(C(=O)OC(C)(C)C)C2)cc1C(=O)O ; NC1(c2cccc3ccccc23)CC1 ; Cc1ccc(NC2CN(C(=O)OC(C)(C)C)C2)cc1C(=O)NC1(c2cccc3ccccc23)CC1 ; Cc1ccc(NC2CNC2)cc1C(=O)NC1(c2cccc3ccccc23)CC1]

### Step 1: tert-Butyl 3-((4-methyl-3-((1-(naphthalen-1-yl)cyclopropyl)carbamoyl)phenyl)amino)azetidine-1-carboxylate (45A-1)

To a solution of 1-(1-naphthyl)cyclopropanamine (39.5 mg, 215 μmol, 1.1 eq) and 5-((1-(tert-butoxycarbonyl)azetidin-3-yl)amino)-2-methylbenzoic acid (60.0 mg, 196 μmol, 1.0 eq) in DCM (4.0 mL) were added TEA (59.5 mg, 588 μmol, 81.8 μL, 3.0 eq), EDCI (113 mg, 588 μmol, 3.0 eq) and HOBt (79.4 mg, 588 μmol, 3.0 eq). The mixture was stirred at 20° C. for 16 h. LCMS indicated that the starting material was completely consumed, and the desired mass was detected. The reaction mixture was poured into H₂O (4.0 mL) and extracted with DCM (2.0 mL×2). The combined organic layers were dried over Na₂SO₄, filtered, and concentrated under vacuum to give the crude product tert-butyl 3-((4-methyl-3-((1-(naphthalen-1-yl)cyclopropyl)carbamoyl)phenyl)amino)azetidine-1-carboxylate (100 mg), which was used in the next step without any further purification. M+H⁺=472.3 (LCMS).

### Step 2: 5-(Azetidin-3-ylamino)-2-methyl-N-(1-(naphthalen-1-yl)cyclopropyl)benzamide (Compound 177)

To a solution of tert-butyl 3-((4-methyl-3-((1-(naphthalen-1-yl)cyclopropyl)carbamoyl)phenyl)amino)azetidine-1-carboxylate (100 mg, 212 μmol, 1.0 eq) in EtOAc (2.0 mL) was added HCl/EtOAc (4 M, 2.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 10%-40% B over 8 min; mobile phase A: 0.04% aqueous HCl, mobile phase B: acetonitrile). 5-(Azetidin-3-ylamino)-2-methyl-N-(1-(naphthalen-1-yl)cyclopropyl)benzamide (21.7 mg, 58.4 μmol, 28% yield, HCl salt) was obtained as a white solid. M+H⁺=372.1 (LCMS); ¹H NMR (400 MHz, DMSO-d₆) δ 9.02-8.88 (m, 3H), 8.68-8.62 (m, 1H), 7.93 (d, J=7.9 Hz, 1H), 7.86-7.76 (m, 2H), 7.62-7.50 (m, 2H), 7.49-7.43 (m, 1H), 6.88 (d, J=8.3 Hz, 1H), 6.41 (dd, J=2.5, 8.1 Hz, 1H), 6.20 (d, J=2.4 Hz, 1H), 4.29-4.20 (m, 1H), 4.31-4.20 (m, 2H), 4.19-4.10 (m, 2H), 1.99-1.83 (m, 3H), 1.39-1.28 (m, 2H), 1.19-1.07 (m, 2H).

### Example 46: 5-(Cyanomethyl)-2-methyl-N-(1-(naphthalen-1-yl)cyclopropyl)benzamide (Compound 313)

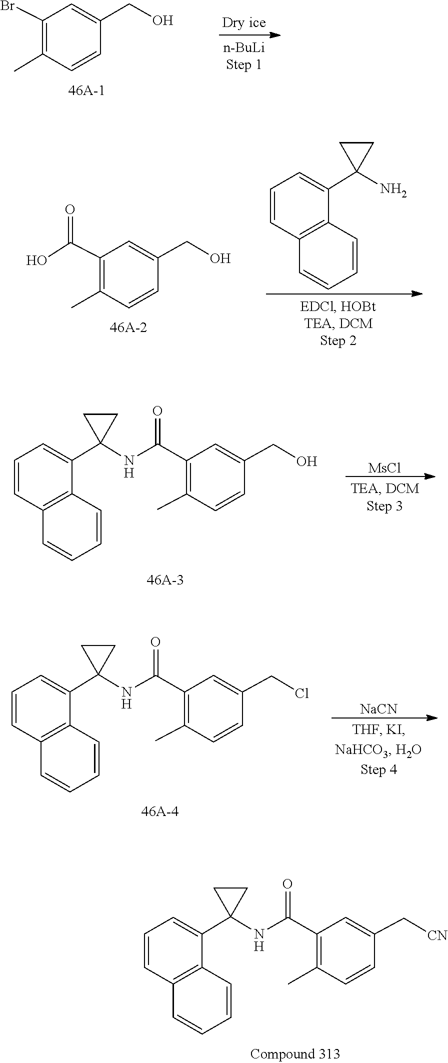

### Step 1: 5-(Hydroxymethyl)-2-methylbenzoic acid (46A-2)

To a solution of (3-bromo-4-methylphenyl) methanol (1.00 g, 4.97 mmol, 1.0 eq) in THF (10 mL) was added n-BuLi (2.5 M in hexane, 4.97 mL, 2.5 eq) at −78° C. The mixture was stirred at the same temperature for 1 h. TLC indicated that the starting material was completely consumed. The reaction mixture was poured into saturated aqueous NH₄Cl (10 mL) and acidified to pH 6 using HCl (1 M, aqueous). The product was extracted with EtOAc (5.0 mL×3). The organic layer was washed with brine (5.0 mL×2). The combined organic layers were dried over Na₂SO₄, filtered, and concentrated under vacuum to give a residue which was purified by trituration from EtOAc/petroleum ether=1/5, then filtered. 5-(Hydroxymethyl)-2-methylbenzoic acid (280 mg, 1.68 mmol, 29% yield) was obtained as a white solid. ¹H NMR (400 MHZ, DMSO-d₆) δ 13.67-12.06 (m, 1H), 7.98 (d, J=0.6 Hz, 1H), 8.04-7.93 (m, 1H), 7.55 (dd, J=1.4, 7.8 Hz, 1H), 7.42 (d, J=7.8 Hz, 1H), 4.68 (s, 2H), 2.68 (s, 3H).

### Step 2: 5-(Hydroxymethyl)-2-methyl-N-(1-(naphthalen-1-yl)cyclopropyl)benzamide (46A-3)

To a solution of 5-(hydroxymethyl)-2-methylbenzoic acid (280 mg, 1.68 mmol, 1.0 eq), 1-(1-naphthyl)cyclopropanamine (309 mg, 1.68 mmol, 1.0 eq) in DMF (10 mL) were added TEA (511 mg, 5.05 mmol, 704 μL, 3.0 eq), EDCI (323 mg, 1.68 mmol, 1.0 eq) and HOBt (228 mg, 1.68 mmol, 1.0 eq). The mixture was stirred at 20° C. for 16 h. LCMS indicated that the starting material was completely consumed, and the desired mass was detected. The reaction mixture was poured into H₂O (10 mL), and a precipitate was formed. The mixture was filtered, and the filter cake was washed with H₂O (5.0 mL) and dried under vacuum to give the crude product 5-(hydroxymethyl)-2-methyl-N-(1-(naphthalen-1-yl)cyclopropyl)benzamide (570 mg) as a white solid. ¹H NMR (400 MHZ, DMSO-d₆) δ 9.12 (s, 1H), 8.66 (d, J=8.2 Hz, 1H), 7.96-7.91 (m, 1H), 7.83 (d, J=7.7 Hz, 2H), 7.60-7.43 (m, 3H), 7.17 (br d, J=7.7 Hz, 1H), 7.07 (d, J=7.8 Hz, 1H), 7.01 (s, 1H), 5.14 (s, 1H), 4.39 (d, J=5.5 Hz, 2H), 2.01 (s, 3H), 1.35 (br s, 2H), 1.17 (br s, 2H).

### Step 3: 5-(Chloromethyl)-2-methyl-N-(1-(naphthalen-1-yl)cyclopropyl)benzamide (46A-4)

To a solution of 5-(hydroxymethyl)-2-methyl-N-(1-(naphthalen-1-yl)cyclopropyl)benzamide (400 mg, 1.21 mmol, 1.0 eq) in DCM (20 mL) was added TEA (122 mg, 1.21 mmol, 168 μL, 1.0 eq), followed by MsCl (138 mg, 1.21 mmol, 93.0 μL, 1.0 eq). The mixture was stirred at 0° C. for 3 h and then at 20° C. for 9 h. LCMS indicated that the desired mass was detected. The reaction mixture was poured into H₂O (20 mL) and extracted with DCM (10 mL×2). The combined organic layers were dried over Na₂SO₄, filtered, and concentrated under vacuum to give a residue which was purified by flash silica gel chromatography using a gradient of EtOAc/petroleum ether from 1/100 to 3/10. 5-(Chloromethyl)-2-methyl-N-(1-(naphthalen-1-yl)cyclopropyl)benzamide (300 mg, 858 μmol, 71% yield) was obtained as a white solid. M+H⁺=350.1 (LCMS); ¹H NMR (400 MHZ, CDCl₃) δ 8.48 (d, J=8.4 Hz, 1H), 7.93 (dd, J=7.6, 16.7 Hz, 2H), 7.82 (d, J=8.3 Hz, 1H), 7.63-7.56 (m, 1H), 7.55-7.45 (m, 2H), 7.25 (s, 1H), 7.15 (d, J=1.3 Hz, 1H), 7.10 (d, J=7.9 Hz, 1H), 6.50 (br s, 1H), 4.47 (s, 2H), 2.17 (s, 3H), 1.55-1.55 (m, 1H), 1.63-1.55 (m, 1H), 1.45-1.38 (m, 2H).

### Step 4: 5-(Cyanomethyl)-2-methyl-N-(1-(naphthalen-1-yl)cyclopropyl)benzamide (Compound 313)

To a solution of 5-(chloromethyl)-2-methyl-N-(1-(naphthalen-1-yl)cyclopropyl)benzamide (300 mg, 858 μmol, 1.0 eq) in THF (9.0 mL) were added NaHCO₃ (1 M aqueous, 15.0 mL), KI (285 mg, 1.72 mmol, 2.0 eq), NaCN (510 mg, 10.4 mmol, 12.0 eq) and H₂O (9.0 mL). The mixture was stirred at 60° C. for 16 h. LCMS indicated that the starting material was completely consumed, and the desired mass was detected. The mixture was allowed to warm to room temperature and concentrated under vacuum at 30° C. to give a residue which was purified by preparative HPLC (Phenomenex Luna C18 column (75×30 mm, 3 μm); flow rate: 25 mL/min; gradient: 30%-80% B over 8 min; mobile phase A: 0.2% aqueous FA, mobile phase B: acetonitrile). 5-(Cyanomethyl)-2-methyl-N-(1-(naphthalen-1-yl)cyclopropyl)benzamide (31.9 mg, 89.0 μmol, 10% yield, FA salt) was obtained as a white solid. M+H⁺=341.1 (LCMS); ¹H NMR (400 MHZ, DMSO-d₆) δ 9.18 (s, 1H), 8.63 (d, J=8.3 Hz, 1H), 7.93 (d, J=7.6 Hz, 1H), 7.83 (dd, J=4.0, 7.6 Hz, 2H), 7.60-7.42 (m, 3H), 7.25-7.19 (m, 1H), 7.18-7.13 (m, 1H), 7.03 (d, J=1.3 Hz, 1H), 3.93 (s, 2H), 1.99 (s, 3H), 1.35 (s, 2H), 1.18 (br s, 2H).

### Example 47: 5-(2-Aminoethyl)-2-methyl-N-(1-(naphthalen-1-yl)cyclopropyl)benzamide (Compound 378)

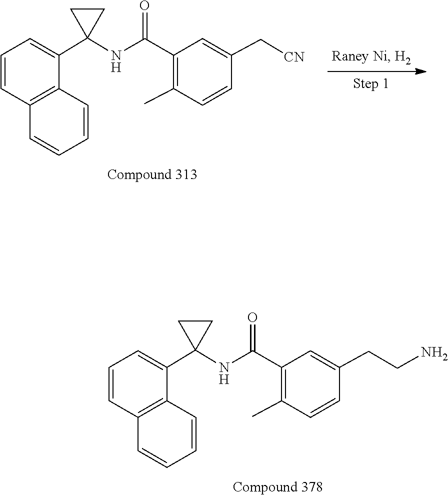 [SMILES: Cc1ccc(CC#N)cc1C(=O)NC1(c2cccc3ccccc23)CC1 ; Cc1ccc(CCN)cc1C(=O)NC1(c2cccc3ccccc23)CC1]

### Step 1: 5-(2-Aminoethyl)-2-methyl-N-(1-(naphthalen-1-yl)cyclopropyl)benzamide (Compound 378)

To a solution of 5-(cyanomethyl)-2-methyl-N-(1-(naphthalen-1-yl)cyclopropyl)benzamide (70.0 mg, 206 μmol, 1.0 eq) in THF (10 mL) were added Raney-Ni (17.6 mg, 206 μmol, 1.0 eq) and NH₃·H₂O (26.7 mg, 206 μmol, 29.3 μL, 27% purity, 1.0 eq) under a N₂ atmosphere. The mixture reaction was degassed under vacuum and purged with H₂ several times. The mixture was stirred under H₂ (50 psi) at 30° C. for 16 h. LCMS indicated that the starting material was completely consumed, and the desired mass was detected. The combined organic layers were filtered through a pad of Celite and the filtrate was concentrated under vacuum to give a residue which was purified by preparative HPLC (Phenomenex Luna C18 column (75×30 mm, 3 μm); flow rate: 25 mL/min; gradient: 20%-40% B over 8 min; mobile phase A: 0.2% aqueous FA, mobile phase B: acetonitrile). 5-(2-Aminoethyl)-2-methyl-N-(1-(naphthalen-1-yl)cyclopropyl)benzamide (17.6 mg, 44.4 μmol, 22% yield, FA salt) was obtained as as a white solid. M+H⁺=345.0 (LCMS); ¹H NMR (400 MHZ, DMSO-d₆) δ 9.14 (s, 1H), 8.66 (br d, J=8.2 Hz, 1H), 8.42 (br s, 1H), 7.93 (d, J=8.1 Hz, 1H), 7.83 (dd, J=2.9, 7.6 Hz, 2H), 7.61-7.44 (m, 3H), 7.13-7.03 (m, 2H), 6.92 (s, 1H), 2.82 (br d, J=6.4 Hz, 2H), 2.68 (br d, J=7.1 Hz, 2H), 2.00 (s, 3H), 1.36 (br s, 2H), 1.18 (br s, 2H).

### Example 48: 6-Methyl-N¹-(1-(naphthalen-1-yl)cyclopropyl) isophthalamide (Compound 257)

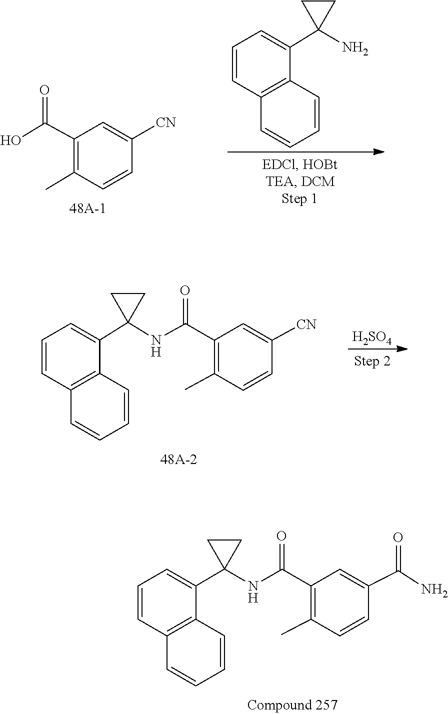

### Step 1: 5-Cyano-2-methyl-N-(1-(naphthalen-1-yl)cyclopropyl)benzamide (48A-2)

To a solution of 1-(naphthalen-1-yl)cyclopropanamine (114 mg, 621 μmol, 1.0 eq) and 5-cyano-2-methylbenzoic acid (100 mg, 621 μmol, 1.0 eq) in DCM (3.0 mL) were added TEA (188 mg, 1.86 mmol, 283 μL, 3.0 eq), EDCI (297 mg, 1.55 mmol, 2.5 eq) and HOBt (210 mg, 1.55 mmol, 2.5 eq). The mixture was stirred at 20° C. for 2 h. LCMS indicated that the starting material was completely consumed, and the desired mass was detected. The reaction mixture was poured into H₂O (3.0 mL) and extracted with DCM (3.0 mL×2). The combined organic layers were dried over Na₂SO₄, filtered, and concentrated under vacuum to give a residue which was purified by flash silica gel chromatography using a gradient of EtOAc/petroleum ether from 0/1 to 1/4. 5-Cyano-2-methyl-N-(1-(naphthalen-1-yl)cyclopropyl)benzamide (150 mg, 460 μmol, 74% yield) was obtained as a yellow oil. M+H⁺=327.2 (LCMS).

### Step 2: 6-Methyl-N¹-(1-(naphthalen-1-yl)cyclopropyl) isophthalamide (Compound 257)

A mixture of 5-cyano-2-methyl-N-(1-(naphthalen-1-yl)cyclopropyl)benzamide (200 mg, 434 μmol, 1.0 eq) in H₂SO₄ (3.0 mL) was stirred at 70° C. for 5 h. LCMS indicated that the starting material was completely consumed, and the desired mass was detected. The mixture was concentrated under vacuum at 30° C. to give a residue which was purified by preparative HPLC (Phenomenex Gemini C18 column (80×40 mm, 3 μm); flow rate: 40 mL/min; gradient: 35%-55% B over 7 min; mobile phase A: 0.04% aqueous HCl, mobile phase B: acetonitrile). 6-Methyl-N¹-(1-(naphthalen-1-yl)cyclopropyl) isophthalamide (16.0 mg, 43.2 μmol, 14% yield, HCl salt) was obtained as a white solid. M+H⁺=345.0 (LCMS); ¹H NMR (400 MHZ, DMSO-d₆) δ 9.19 (s, 1H), 8.64 (s, 1H), 7.98-7.88 (m, 2H), 7.84 (d, J=7.9 Hz, 2H), 7.74 (br d, J=7.9 Hz, 1H), 7.64-7.43 (m, 4H), 7.27 (br s, 1H), 7.21 (d, J=8.0 Hz, 1H), 2.06 (s, 3H), 1.38 (s, 2H), 1.19 (br s, 2H).

### Example 49: 5-(2-Amino-2-oxoethyl)-2-methyl-N-(1-(naphthalen-1-yl)cyclopropyl)benzamide (Compound 312)

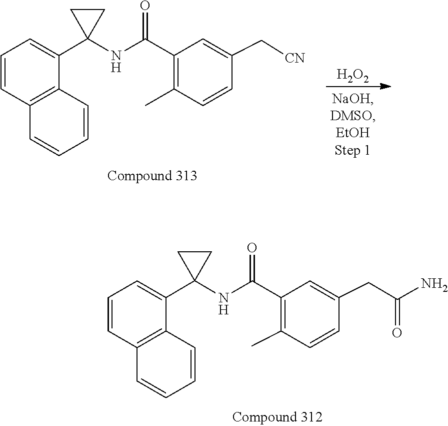

### Step 1: 5-(2-Amino-2-oxoethyl)-2-methyl-N-(1-(naphthalen-1-yl)cyclopropyl)benzamide (Compound 312)

To a solution of 5-(cyanomethyl)-2-methyl-N-(1-(naphthalen-1-yl)cyclopropyl)benzamide (100 mg, 294 μmol, 1.0 eq) in a mixture of DMSO (1.0 mL) and EtOH (3.0 mL) were added H₂O₂ (99.9 mg, 881 μmol, 84.7 μL, 30% purity, 3.0 eq) and NaOH (2 M aqueous, 441 μL, 3.0 eq). The mixture was stirred at 20° C. for 1 h. LCMS indicated that the starting material was completely consumed, and the desired mass was detected. The reaction mixture was poured into saturated aqueous NaSO₃ (20 mL) and extracted with EtOAc (15 mL×3). The combined organic layers were dried over Na₂SO₄, filtered, and concentrated under vacuum to give a residue which was purified by trituration from EtOAc/petroleum ether=1/5, then filtered. 5-(2-Amino-2-oxoethyl)-2-methyl-N-(1-(naphthalen-1-yl)cyclopropyl)benzamide (35.2 mg, 89.7 μmol, 31% yield) was obtained as a white solid. M+H⁺=359.0 (LCMS); ¹H NMR (400 MHz, DMSO-d₆) δ 9.11 (s, 1H), 8.64 (br d, J=8.4 Hz, 1H), 7.93 (br d, J=7.9 Hz, 1H), 7.83 (br d, J=7.7 Hz, 2H), 7.60-7.44 (m, 3H), 7.38 (br s, 1H), 7.13 (br d, J=7.6 Hz, 1H), 7.07-7.02 (m, 1H), 6.95 (s, 1H), 6.81 (br s, 1H), 3.25 (s, 2H), 1.96 (s, 3H), 1.35 (br s, 2H), 1.17 (br s, 2H).

### Example 50: 5-(2,5-Dihydro-1H-pyrrol-3-yl)-2-methyl-N-(1-(naphthalen-1-yl)cyclopropyl)benzamide (Compound 398)

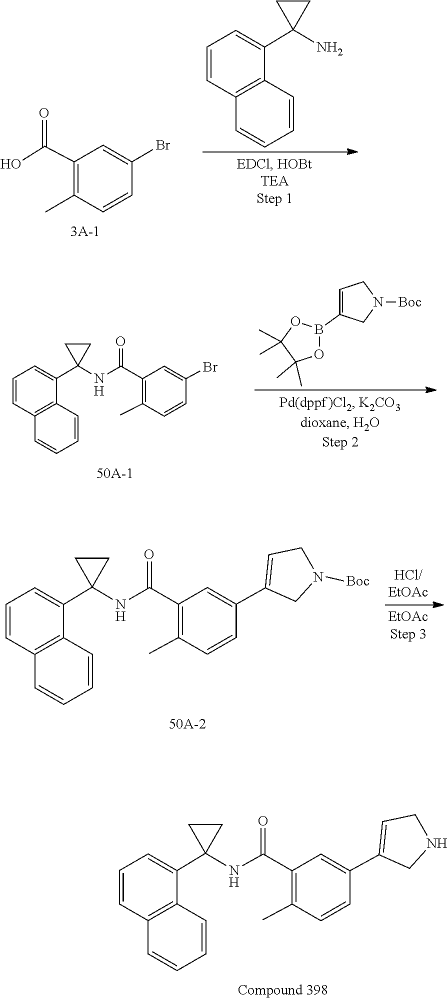 [SMILES: Cc1ccc(Br)cc1C(=O)O ; NC1(c2cccc3ccccc23)CC1 ; Cc1ccc(Br)cc1C(=O)NC1(c2cccc3ccccc23)CC1 ; CC(C)(C)OC(=O)N1CC=C(B2OC(C)(C)C(C)(C)O2)C1 ; Cc1ccc(C2=CCN(C(=O)OC(C)(C)C)C2)cc1C(=O)NC1(c2cccc3ccccc23)CC1 ; Cc1ccc(C2=CCNC2)cc1C(=O)NC1(c2cccc3ccccc23)CC1]

### Step 1: 5-Bromo-2-methyl-N-(1-(naphthalen-1-yl)cyclopropyl)benzamide (50A-1)

To a solution of 1-(naphthalen-1-yl)cyclopropanamine (155 mg, 846 μmol, 1.0 eq) and 5-bromo-2-methylbenzoic acid (200 mg, 930 μmol, 1.1 eq) in DMF (2.0 mL) were added TEA (257 mg, 2.54 mmol, 353 μL, 3.0 eq), EDCI (243 mg, 1.27 mmol, 1.5 eq) and HOBt (171 mg, 1.27 mmol, 1.5 eq). The mixture was stirred at 25° C. for 16 h. LCMS indicated that the starting material was completely consumed, and the desired mass was detected. The reaction mixture was poured into H₂O (10 mL) and extracted with EtOAc (10 mL×2). The combined organic layers were dried over Na₂SO₄, filtered, and concentrated under vacuum to give a residue which was purified by preparative TLC (EtOAc/petroleum ether=1/2, R_{f}=0.7). 5-Bromo-2-methyl-N-(1-(naphthalen-1-yl)cyclopropyl)benzamide (260 mg, 684 μmol, 81% yield) was obtained as a white solid. M+H⁺=380.0 (LCMS); ¹H NMR (400 MHZ, DMSO-d₆) δ 9.25 (s, 1H), 8.63 (d, J=8.2 Hz, 1H), 7.94 (d, J=8.2 Hz, 1H), 7.82 (dd, J=7.8, 11.1 Hz, 2H), 7.62-7.50 (m, 2H), 7.48-7.39 (m, 2H), 7.21 (d, J=1.8 Hz, 1H), 7.10 (d, J=8.2 Hz, 1H), 1.97 (s, 3H), 1.37 (br s, 2H), 1.17 (br s, 2H).

### Step 2: tert-Butyl 3-(4-methyl-3-((1-(naphthalen-1-yl)cyclopropyl)carbamoyl)phenyl)-2,5-dihydro-1H-pyrrole-1-carboxylate (50A-2)

To a stirred solution of 5-bromo-2-methyl-N-(1-(naphthalen-1-yl)cyclopropyl)benzamide (60.0 mg, 158 μmol, 1.0 eq) and tert-butyl 3-(4,4,5,5-tetramethyl-1,3,2-dioxaborolan-2-yl)-2,5-dihydro-1H-pyrrole-1-carboxylate (55.9 mg, 189 μmol, 1.2 eq) in a mixture of dioxane (1.0 mL) and H₂O (0.3 mL) were added Pd(dppf)Cl₂ (11.5 mg, 15.8 μmol, 0.1 eq), K₂CO₃ (65.4 mg, 473 μmol, 3.0 eq) in one portion. The mixture was degassed and purged with N₂ three times and then was stirred at 80° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by preparative TLC (EtOAc/petroleum ether=1/2, R_{f}=0.4). tert-Butyl 3-(4-methyl-3-((1-(naphthalen-1-yl)cyclopropyl)carbamoyl)phenyl)-2,5-dihydro-1H-pyrrole-1-carboxylate (70.0 mg, 149 μmol, 95% yield) was obtained as a white solid. M+H⁺=469.2 (LCMS).

### Step 3: 5-(2,5-Dihydro-1H-pyrrol-3-yl)-2-methyl-N-(1-(naphthalen-1-yl)cyclopropyl)benzamide (Compound 398)

To a stirred solution of tert-butyl 3-(4-methyl-3-((1-(naphthalen-1-yl)cyclopropyl)carbamoyl)phenyl)-2,5-dihydro-1H-pyrrole-1-carboxylate (70.0 mg, 149 μmol, 1.0 eq) in EtOAc (1.0 mL) was added HCl/EtOAc (4 M, 5.0 mL). The mixture was stirred at 20° C. for 4 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 1%-60% B over 8 min; mobile phase A: 0.04% aqueous HCl, mobile phase B: acetonitrile). 5-(2,5-Dihydro-1H-pyrrol-3-yl)-2-methyl-N-(1-(naphthalen-1-yl)cyclopropyl)benzamide (25.3 mg, 68.7 μmol, 46% yield, HCl salt) was obtained as a white solid. M+H⁺=369.1 (LCMS); ¹H NMR (400 MHZ, DMSO-d₆) δ 9.45 (br s, 2H), 9.16 (s, 1H), 8.66 (d, J=8.4 Hz, 1H), 7.98-7.91 (m, 1H), 7.86-7.79 (m, 2H), 7.62-7.55 (m, 1H), 7.54 (br d, J=1.4 Hz, 1H), 7.49-7.40 (m, 2H), 7.21-7.12 (m, 2H), 6.34 (t, J=1.8 Hz, 1H), 4.25 (br d, J=1.8 Hz, 2H), 4.10 (br s, 2H), 2.03 (s, 3H), 1.42-1.34 (m, 2H), 1.24-1.15 (m, 2H).

### Example 51: 2-Methyl-N-(1-(naphthalen-1-yl)cyclopropyl)-5-(1,2,3,6-tetrahydropyridin-4-yl)benzamide (Compound 399)

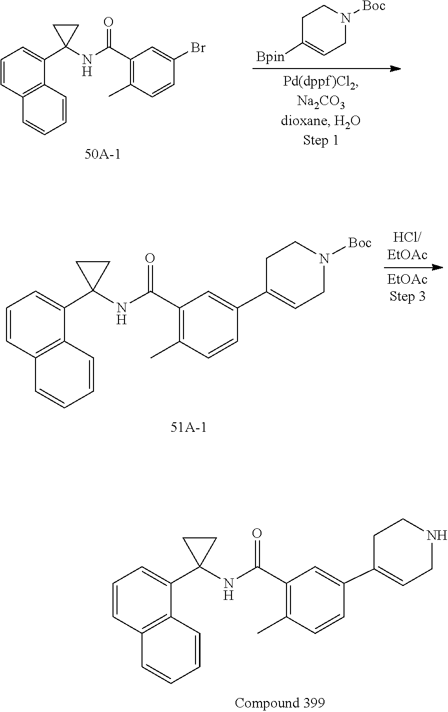

### Step 1: tert-Butyl 4-(4-methyl-3-((1-(naphthalen-1-yl)cyclopropyl)carbamoyl)phenyl)-5,6-dihydropyridine-1 (2H)-carboxylate (51A-1)

To a stirred solution of 5-bromo-2-methyl-N-(1-(naphthalen-1-yl)cyclopropyl)benzamide (60.0 mg, 158 μmol, 1.0 eq) and tert-butyl 4-(4,4,5,5-tetramethyl-1,3,2-dioxaborolan-2-yl)-5,6-dihydropyridine-1 (2H)-carboxylate (58.5 mg, 189 μmol, 1.2 eq) in a mixture of dioxane (1.0 mL) and H₂O (0.3 mL) was added Pd(dppf)Cl₂ (11.5 mg, 15.8 μmol, 0.1 eq), K₂CO₃ (65.4 mg, 473 μmol, 3.0 eq) in one portion. The mixture was degassed and purged with N₂ three times and then was stirred at 80° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by preparative TLC (EtOAc/petroleum ether=1/2, R_{f}=0.4). tert-Butyl 4-(4-methyl-3-((1-(naphthalen-1-yl)cyclopropyl)carbamoyl)phenyl)-5,6-dihydropyridine-1 (2H)-carboxylate (70.0 mg, 145 μmol, 92% yield) was obtained as a white solid. M+H⁺=483.2 (LCMS).

### Step 2: 2-Methyl-N-(1-(naphthalen-1-yl)cyclopropyl)-5-(1,2,3,6-tetrahydropyridin-4-yl)benzamide (Compound 399)

To a stirred solution of tert-butyl 4-(4-methyl-3-((1-(naphthalen-1-yl)cyclopropyl)carbamoyl)phenyl)-5,6-dihydropyridine-1 (2H)-carboxylate (70.0 mg, 145 μmol, 1.0 eq) in EtOAc (1.0 mL) was added HCl/EtOAc (4 M, 5.0 mL). The mixture was stirred at 20° C. for 4 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 1%-60% B over 8 min; mobile phase A: 0.04% aqueous HCl, mobile phase B: acetonitrile). 2-Methyl-N-(1-(naphthalen-1-yl)cyclopropyl)-5-(1,2,3,6-tetrahydropyridin-4-yl)benzamide (50.0 mg, 131 μmol, 90% yield, HCl salt) was obtained as a white solid. M+H⁺=383.1 (LCMS); ¹H NMR (400 MHZ, DMSO-d₆) δ 9.17 (s, 1H), 9.11 (br s, 2H), 8.66 (d, J=8.3 Hz, 1H), 7.94 (d, J=7.9 Hz, 1H), 7.83 (t, J=7.1 Hz, 2H), 7.61-7.55 (m, 1H), 7.55-7.49 (m, 1H), 7.47 (t, J=7.6 Hz, 1H), 7.36 (dd, J=1.8, 8.1 Hz, 1H), 7.19-7.09 (m, 2H), 6.10 (br s, 1H), 3.70 (br s, 2H), 3.27 (br s, 2H), 2.58 (br s, 2H), 2.03 (s, 3H), 1.42-1.33 (m, 2H), 1.24-1.14 (m, 2H).

### Example 52: (E)-Methyl 4-(2-(3-(2-((1-(naphthalen-1-yl)cyclopropyl)carbamoyl)phenyl)propanoyl) hydrazinyl)-4-oxobut-2-enoate (Compound 373)

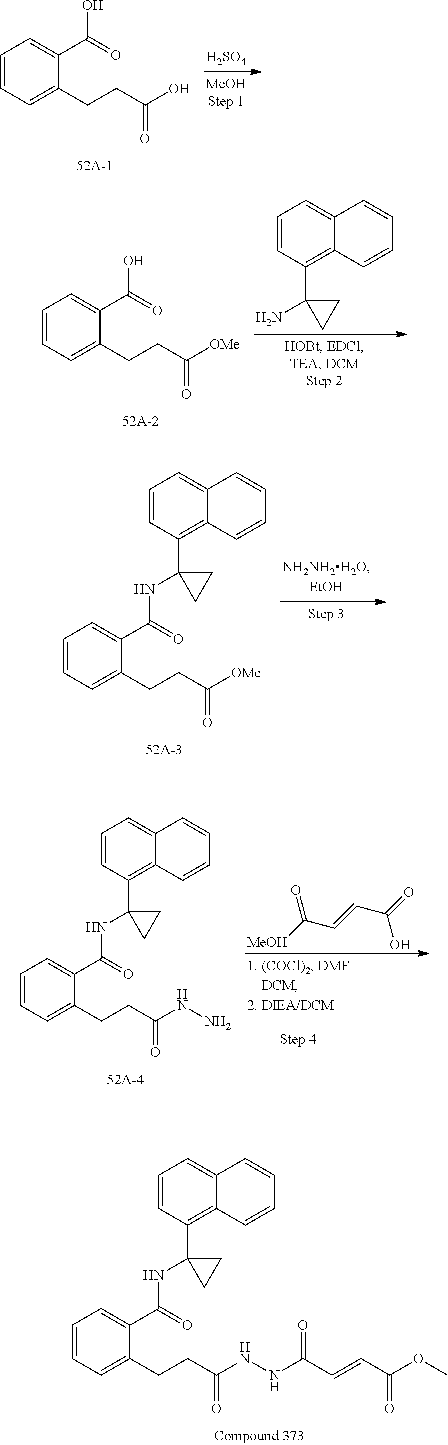

### Step 1: 2-(3-Methoxy-3-oxopropyl)benzoic acid (52A-2)

To a solution of 2-(2-carboxyethyl)benzoic acid (2.00 g, 10.0 mmol, 1.0 eq) in MeOH (30 mL) was added H₂SO₄ (1.00 g, 10.0 mmol, 500 μL, 1.0 eq). The mixture was stirred at 25° C. for 1 h. LCMS indicated that the starting material was completely consumed, and the desired mass was detected. The reaction mixture was poured into H₂O (30 mL) and extracted with EtOAc (10 mL×3). The reaction mixture was concentrated under vacuum to give a residue which was purified by flash silica gel chromatography using a gradient of EtOAc/petroleum ether from 0/1 to 1/3. 2-(3-Methoxy-3-oxo-propyl)benzoic acid (1.50 g, 7.00 mmol, 70% yield) was obtained as a white solid. M+H⁺=209.2 (LCMS); ¹H NMR (400 MHZ, CD₃OD) δ 7.93 (d, 1H), 7.44-7.50 (m, 1H), 7.28-7.35 (m, 2H), 3.65 (s, 3H), 3.28-3.34 (m, 2H), 2.66 (t, 2H).

### Step 2: Methyl 3-(2-((1-(naphthalen-1-yl)cyclopropyl)carbamoyl)phenyl)propanoate (52A-3)

To a solution of 2-(3-methoxy-3-oxo-propyl)benzoic acid (300 mg, 1.44 mmol, 1.0 eq) and 1-(1-naphthyl)cyclopropanamine (264 mg, 1.44 mmol, 1.0 eq) in DCM (10 mL) were added TEA (437 mg, 4.32 mmol, 601 μL, 3.0 eq), HOBt (292 mg, 2.16 mmol, 1.5 eq) and EDCI (414 Mg, 2.16 mmol, 1.5 eq). The mixture was stirred at 25° C. for 1 h. LCMS indicated that the starting material was completely consumed, and the desired mass was detected. The reaction mixture was concentrated under vacuum to give a residue which was purified by preparative HPLC (Phenomenex C18 column (75×30 mm, 3 μm); flow rate: 25 mL/min; gradient: 30%-60% B over 8 min; mobile phase A: 10 mM aqueous NH₄HCO₃, mobile phase B: acetonitrile). Methyl 3-(2-((1-(naphthalen-1-yl)cyclopropyl)carbamoyl)phenyl)propanoate (109 mg, 300 μmol, 20% yield) was obtained a white solid. M+H⁺=374.2 (LCMS).

### Step 3: 2-(3-Hydrazinyl-3-oxopropyl)-N-(1-(naphthalen-1-yl)cyclopropyl)benzamide (52A-4)

To a mixture of methyl 3-(2-((1-(naphthalen-1-yl)cyclopropyl)carbamoyl)phenyl)propanoate (90.0 mg, 200 μmol, 1.0 eq) in EtOH (3.0 mL) was added NH₂NH₂·H₂O (120 mg, 2.00 mmol, 100 μL, 10 eq) at 25° C. The reaction mixture was stirred at 80° C. for 5 h. TLC indicated that the starting material was completely consumed. The reaction mixture was allowed to cool to room temperature, concentrated under vacuum to give the crude product 2-(3-hydrazino-3-oxo-propyl)-N-[1-(1-naphthyl)cyclopropyl]benzamide (90.0 mg), which was used in the next step without any further purification. M+H⁺=374.2 (LCMS).

### Step 4: (E)-Methyl 4-(2-(3-(2-((1-(naphthalen-1-yl)cyclopropyl)carbamoyl)phenyl)propanoyl)hydrazinyl)-4-oxobut-2-enoate (Compound 373)

To a mixture of (E)-4-methoxy-4-oxo-but-2-enoic acid (200 mg, 1.54 mmol, 1.0 eq) and DMF (11.2 mg, 154 μmol, 11.8 μL, 0.1 eq) in DCM (1.0 mL) was added (COCl)₂ (390 mg, 3.07 mmol, 269 μL, 2.0 eq) at 0° C. The reaction mixture was stirred at 0° C. for 15 min. The reaction mixture was concentrated under vacuum to give a residue which was diluted with DCM (500 μL). The solution was added to a mixture of 2-(3-hydrazino-3-oxo-propyl)-N-[1-(1-naphthyl)cyclopropyl]benzamide (90.0 mg, 241 μmol, 1.0 eq) and DIEA (156 mg, 1.20 mmol, 210 μL, 5.0 eq) in DCM (1.0 mL) at 0° C. The reaction mixture was stirred at 25° C. for 15 min. LCMS indicated that the starting material was completely consumed, and the desired mass was detected. The reaction mixture was concentrated under vacuum to give a residue which was purified by preparative HPLC (Waters Xbridge BEH C18 column (100×30 mm, 10 μm); flow rate: 25 mL/min; gradient: 30%-60% B over 8 min; mobile phase A: 10 mM aqueous NH₄HCO₃, mobile phase B: acetonitrile). (E)-Methyl 4-(2-(3-(2-((1-(naphthalen-1-yl)cyclopropyl)carbamoyl)phenyl)propanoyl) hydrazinyl)-4-oxobut-2-enoate (11.9 mg, 23.8 μmol, 11% yield) was obtained as a white solid. M+H⁺=486.1 (LCMS); ¹H NMR (400 MHZ, DMSO-d₆) δ 9.22 (s, 1H), 8.66 (br d, J=8.4 Hz, 1H), 7.93 (br d, J=7.8 Hz, 1H), 7.82 (br dd, J=2.4, 7.6 Hz, 2H), 7.62-7.42 (m, 3H), 7.32-7.18 (m, 2H), 7.17-6.96 (m, 3H), 6.69 (d, J=15.6 Hz, 1H), 3.75 (s, 3H), 2.78 (br t, J=7.8 Hz, 2H), 2.38 (br t, J=7.8 Hz, 2H), 1.39 (br s, 2H), 1.17 (br s, 2H).

### Example 53: 5-(2-Aminoethoxy)-2-methyl-N-(1-(naphthalen-1-yl)cyclopropyl)benzamide (Compound 215)

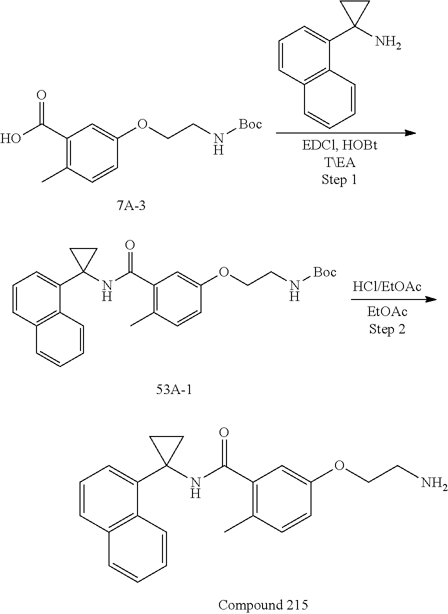 [SMILES: Cc1ccc(OCCNC(=O)OC(C)(C)C)cc1C(=O)O ; NC1(c2cccc3ccccc23)CC1 ; Cc1ccc(OCCNC(=O)OC(C)(C)C)cc1C(=O)NC1(c2cccc3ccccc23)CC1 ; Cc1ccc(OCCN)cc1C(=O)NC1(c2cccc3ccccc23)CC1]

### Step 1: tert-Butyl(2-(4-methyl-3-((1-(naphthalen-1-yl)cyclopropyl)carbamoyl)phenoxy)ethyl)carbamate (53A-1)

To a solution of 1-(naphthalen-1-yl)cyclopropanamine (124 mg, 677 μmol, 1.0 eq) and 5-(2-((tert-butoxycarbonyl)amino)ethoxy)-2-methylbenzoic acid (200 mg, 677 μmol, 1.0 eq) in DCM (10 mL) were added TEA (206 mg, 2.03 mmol, 283 μL, 3.0 eq), EDCI (325 mg, 1.69 mmol, 2.5 eq) and HOBt (229 mg, 1.69 mmol, 2.5 eq). The mixture was stirred at 20° C. for 16 h. LCMS indicated that the starting material was completely consumed, and the desired mass was detected. The reaction mixture was poured into H₂O (10 mL) and extracted with DCM (10 mL×2). The combined organic layers were dried over Na₂SO₄, filtered, and concentrated under vacuum to give a residue which was purified by flash silica gel chromatography using a gradient of EtOAc/petroleum ether from 1/100 to 1/1. tert-Butyl(2-(4-methyl-3-((1-(naphthalen-1-yl)cyclopropyl)carbamoyl)phenoxy)ethyl)carbamate (300 mg, 651 μmol, 96% yield) was obtained as a yellow gum. M+H⁺=461.2 (LCMS); ¹H NMR (400 MHZ, CDCl₃) δ 8.47 (br d, J=8.3 Hz, 1H), 7.93 (br dd, J=7.6, 17.4 Hz, 2H), 7.81 (br d, J=8.2 Hz, 1H), 7.62-7.55 (m, 1H), 7.54-7.45 (m, 2H), 7.00 (br d, J=8.6 Hz, 1H), 6.78-6.72 (m, 1H), 6.69 (br s, 1H), 6.50 (br s, 1H), 3.95-3.83 (m, 2H), 3.46 (br d, J=4.8 Hz, 2H), 1.50-1.36 (m, 13H).

### Step 2: 5-(2-Aminoethoxy)-2-methyl-N-(1-(naphthalen-1-yl)cyclopropyl)benzamide (Compound 215)

To a stirred solution of tert-butyl(2-(4-methyl-3-((1-(naphthalen-1-yl)cyclopropyl)carbamoyl) phenoxy)ethyl)carbamate (200 mg, 434 μmol, 1.0 eq) in EtOAc (3.0 mL) was added HCl/EtOAc (4 M, 3.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Gemini C18 column (75×30 mm, 3 μm); flow rate: 25 mL/min; gradient: 10%-40% B over 8 min; mobile phase A: 0.04% aqueous HCl, mobile phase B: acetonitrile) to give 5-(2-aminoethoxy)-2-methyl-N-(1-(naphthalen-1-yl)cyclopropyl)benzamide (140 mg, 334 μmol, 77% yield, HCl salt) as a white solid. M+H⁺=361.1 (LCMS); ¹H NMR (400 MHZ, DMSO-d₆) δ 9.14 (s, 1H), 8.66 (d, J=8.3 Hz, 1H), 8.13 (br s, 3H), 7.93 (d, J=7.5 Hz, 1H), 7.86-7.78 (m, 2H), 7.62-7.42 (m, 3H), 7.07 (d, J=8.5 Hz, 1H), 6.88 (dd, J=2.6, 8.4 Hz, 1H), 6.67 (d, J=2.8 Hz, 1H), 4.09 (t, J=5.1 Hz, 2H), 3.17-3.08 (m, 2H), 1.97 (s, 3H), 1.39-1.33 (m, 2H), 1.21-1.13 (m, 2H).

### Example 54: 5-(2-Aminoethoxy)-N,2-dimethyl-N-(1-(naphthalen-1-yl)cyclopropyl)benzamide (Compound 211)

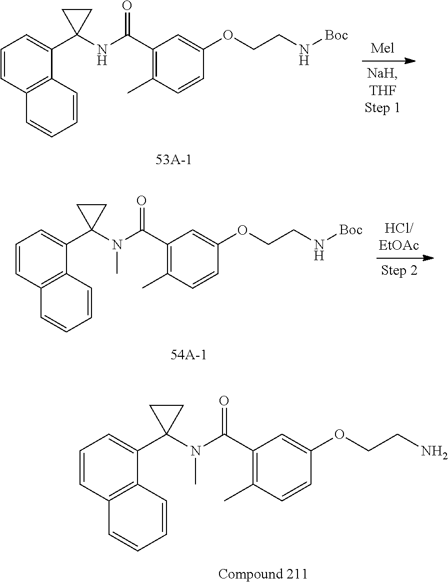 [SMILES: Cc1ccc(OCCNC(=O)OC(C)(C)C)cc1C(=O)NC1(c2cccc3ccccc23)CC1 ; Cc1ccc(OCCNC(=O)OC(C)(C)C)cc1C(=O)N(C)C1(c2cccc3ccccc23)CC1 ; Cc1ccc(OCCN)cc1C(=O)N(C)C1(c2cccc3ccccc23)CC1]

### Step 1: tert-Butyl(2-(4-methyl-3-(methyl(1-(naphthalen-1-yl)cyclopropyl)carbamoyl)phenoxy)ethyl)carbamate (54A-1)

To a solution of tert-butyl(2-(4-methyl-3-((1-(naphthalen-1-yl)cyclopropyl)carbamoyl)phenoxy)ethyl)carbamate (50.0 mg, 109 μmol, 1.0 eq) in THF (3.0 mL) was added sodium hydride (8.68 mg, 217 μmol, 60% purity, 2.0 eq) and the mixture was stirred at 0° C. for 30 min. Mel (7.70 mg, 54.3 μmol, 3.38 μL, 0.5 eq) was added at 0° C. and was stirred at 20° C. for 2 h under a N₂ atmosphere. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 50%-80% B over 8 min; mobile phase A: 0.04% aqueous HCl, mobile phase B: acetonitrile). tert-Butyl(2-(4-methyl-3-(methyl(1-(naphthalen-1-yl)cyclopropyl)carbamoyl)phenoxy)ethyl)carbamate (50.0 mg, 105 μmol, 97% yield, HCl salt) was obtained as a white solid. M+H⁺=475.1. Step 2: 5-(2-Aminoethoxy)-N,2-dimethyl-N-(1-(naphthalen-1-yl)cyclopropyl)benzamide (Compound 211)

To a solution of tert-butyl(2-(4-methyl-3-(methyl(1-(naphthalen-1-yl)cyclopropyl)carbamoyl) phenoxy)ethyl)carbamate (50.0 mg, 105 μmol, 1.0 eq) in EtOAc (2.0 mL) was added HCl/EtOAc (4 M, 2.91 mL). The mixture was stirred at 20° C. for 1 h. LCMS indicated that the starting material was completely consumed, and the desired mass was detected. The mixture was concentrated under vacuum at 30° C. to give 5-(2-aminoethoxy)-N,2-dimethyl-N-(1-(naphthalen-1-yl)cyclopropyl)benzamide (38.0 mg, 84.2 μmol, 76% yield, HCl salt) as a white solid. M+H⁺=375.1; 1H NMR (400 MHZ, DMSO-d₆) δ 9.20-9.07 (m, 1H), 8.08 (br s, 2H), 8.14-8.01 (m, 1H), 7.99-7.85 (m, 3H), 7.64-7.47 (m, 3H), 7.13-7.07 (m, 1H), 6.90-6.82 (m, 1H), 6.62-6.54 (m, 1H), 4.15-4.06 (m, 2H), 3.16-3.09 (m, 2H), 2.78-2.71 (m, 3H), 1.86-1.81 (m, 5H), 1.37 (br s, 2H).

### Example 55: 5-(3-Aminopropoxy)-2-methyl-N-(1-(naphthalen-1-yl)cyclopropyl)benzamide (Compound 316)

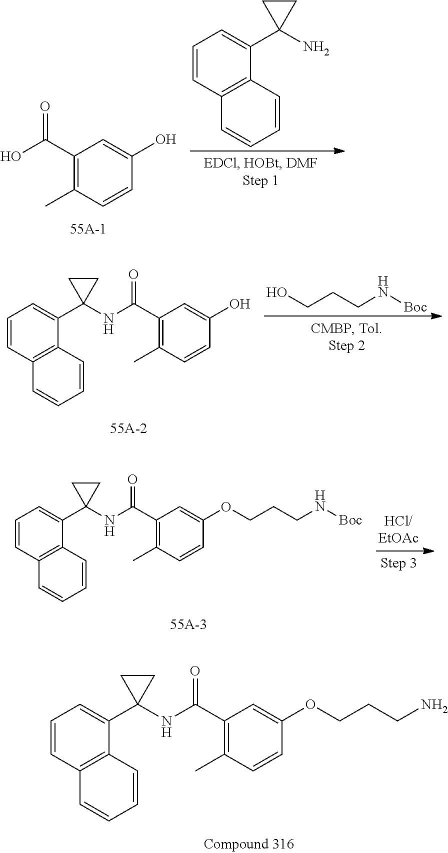 [SMILES: Cc1ccc(O)cc1C(=O)O ; NC1(c2cccc3ccccc23)CC1 ; Cc1ccc(O)cc1C(=O)NC1(c2cccc3ccccc23)CC1 ; CC(C)(C)OC(=O)NCCCO ; Cc1ccc(OCCCNC(=O)OC(C)(C)C)cc1C(=O)NC1(c2cccc3ccccc23)CC1 ; Cc1ccc(OCCCN)cc1C(=O)NC1(c2cccc3ccccc23)CC1]

### Step 1: 5-Hydroxy-2-methyl-N-(1-(naphthalen-1-yl)cyclopropyl)benzamide (55A-2)

To a solution of 1-(naphthalen-1-yl)cyclopropanamine (4.82 g, 26.3 mmol, 0.8 eq) and 5-hydroxy-2-methylbenzoic acid (5.00 g, 32.9 mmol, 1.0 eq) in DMF (100 mL) were added TEA (3.33 g, 32.7 mmol, 4.57 mL, 1.0 eq), EDCI (6.61 g, 34.5 mmol, 1.1 eq) and HOBt (888 mg, 6.57 mmol, 0.2 eq). The mixture was stirred at 25° C. for 4 h. LCMS indicated that the starting material was completely consumed, and the desired mass was detected. The reaction mixture was poured into H₂O (100 mL) and extracted with EtOAc (20 mL×5). The combined organic layers were dried over Na₂SO₄, filtered, and concentrated under vacuum to give a residue which was triturated from a mixture of EtOAc (10 mL) and petroleum ether (30 mL) and stirred at room temperature for 10 min. 5-Hydroxy-2-methyl-N-(1-(naphthalen-1-yl)cyclopropyl)benzamide (5.00 g, 15.8 mmol, 48% yield) was obtained as a white solid. M+H⁺=318.1 (LCMS).

### Step 2: tert-Butyl(3-(4-methyl-3-((1-(naphthalen-1-yl)cyclopropyl)carbamoyl)phenoxy) propyl)carbamate (55A-3)

A mixture of 5-hydroxy-2-methyl-N-(1-(naphthalen-1-yl)cyclopropyl)benzamide (100 mg, 315 μmol, 1.0 eq) and tert-butyl(3-hydroxypropyl)carbamate (55.2 mg, 315 μmol, 54.1 μL, 1.0 eq) in anhydrous toluene (5.0 mL) was degassed and purged with N₂ three times. To the mixture was added CMBP (114 mg, 473 μmol, 1.5 eq) dropwise at 20° C. The mixture was stirred at 110° C. for 16 h. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by preparative TLC (EtOAc/petroleum ether=1/1, R_{f}=0.5). tert-Butyl(3-(4-methyl-3-((1-(naphthalen-1-yl)cyclopropyl)carbamoyl)phenoxy) propyl) carbamate (70.0 mg, 148 μmol, 47% yield, HCl salt) was obtained as a white solid. M+H⁺=475.2 (LCMS); ¹H NMR (400 MHZ, DMSO-d₆) δ 9.07 (s, 1H), 8.66 (d, J=8.3 Hz, 1H), 7.93 (d, J=7.9 Hz, 1H), 7.86-7.79 (m, 2H), 7.60-7.43 (m, 3H), 7.02 (d, J=8.4 Hz, 1H), 6.88-6.78 (m, 2H), 6.60 (d, J=2.6 Hz, 1H), 3.86 (t, J=6.3 Hz, 2H), 3.02 (q, J=6.5 Hz, 2H), 1.95 (s, 3H), 1.76 (q, J=6.5 Hz, 2H), 1.35 (s, 11H), 1.19-1.16 (m, 2H).

### Step 3: 5-(3-Aminopropoxy)-2-methyl-N-(1-(naphthalen-1-yl)cyclopropyl)benzamide (Compound 316)

To a stirred solution of tert-butyl(3-(4-methyl-3-((1-(naphthalen-1-yl)cyclopropyl)carbamoyl) phenoxy) propyl)carbamate (70.0 mg, 148 μmol, 1.0 eq) in EtOAc (2.0 mL) was added HCl/EtOAc (4 M, 6.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 5%-35% B over 8 min; mobile phase A: 0.04% aqueous HCl, mobile phase B: acetonitrile). 5-(3-Aminopropoxy)-2-methyl-N-(1-(naphthalen-1-yl)cyclopropyl)benzamide (53.7 mg, 131 μmol, 87% yield, HCl salt) was obtained as a white solid. M+H⁺=375.1 (LCMS); ¹H NMR (400 MHZ, DMSO-d₆) δ 9.09 (s, 1H), 8.66 (d, J=8.4 Hz, 1H), 7.98-7.85 (m, 4H), 7.85-7.80 (m, 2H), 7.60-7.49 (m, 2H), 7.46 (dd, J=7.3, 8.1 Hz, 1H), 7.04 (d, J=8.6 Hz, 1H), 6.84 (dd, J=2.8, 8.4 Hz, 1H), 6.63 (d, J=2.6 Hz, 1H), 3.96 (t, J=6.2 Hz, 2H), 2.94-2.83 (m, 2H), 1.99-1.91 (m, 5H), 1.38-1.32 (m, 2H), 1.21-1.12 (m, 2H).

### Example 56: 2-Methyl-5-(2-(methylamino)ethoxy)-N-(1-(naphthalen-1-yl)cyclopropyl)benzamide (Compound 233)

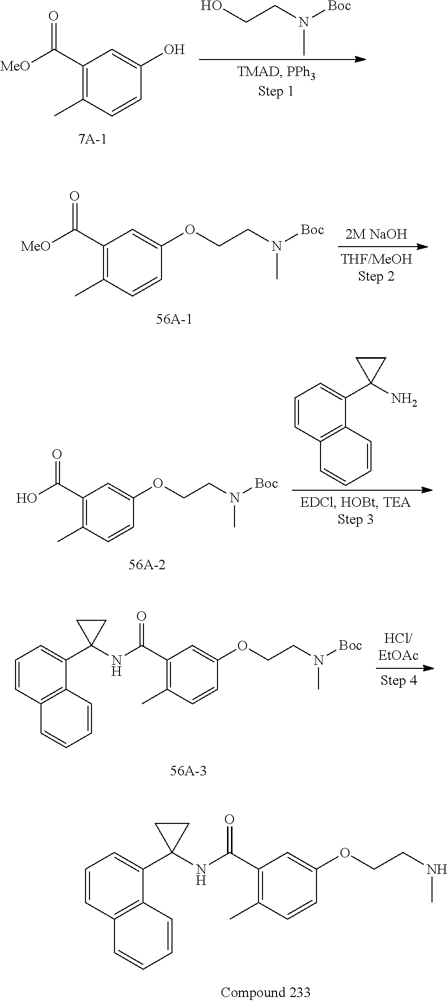

### Step 1: Methyl 5-(2-((tert-butoxycarbonyl)(methyl)amino)ethoxy)-2-methylbenzoate (56A-1)

To a solution of methyl 5-hydroxy-2-methylbenzoate (1.00 g, 6.02 mmol, 1.0 eq) and tert-butyl(2-hydroxyethyl)(methyl)carbamate (1.58 g, 9.03 mmol, 1.5 eq) in toluene (30 mL) were added TMAD (3.11 g, 18.1 mmol, 3.0 eq) and PPh₃ (4.74 g, 18.1 mmol, 3.0 eq). The mixture was degassed and purged with N₂ three times, and then stirred at 100° C. for 16 h under a N₂ atmosphere. LCMS indicated that the starting material was completely consumed, and the desired mass was detected. The reaction mixture was allowed to cool to room temperature, poured into H₂O (30 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/100 to 1/5. Methyl 5-(2-((tert-butoxycarbonyl)(methyl)amino)ethoxy)-2-methylbenzoate (600 mg, 1.86 mmol, 31% yield) was obtained as a yellow oil. M−56+H⁺=268.1 (LCMS); ¹H NMR (400 MHZ, CDCl₃) δ 7.43 (br s, 1H), 7.14 (d, J=8.4 Hz, 1H), 6.95 (dd, J=2.4, 8.3 Hz, 1H), 4.16-4.04 (m, 2H), 3.89 (s, 3H), 3.60 (br s, 2H), 2.98 (s, 3H), 2.52 (s, 3H), 1.46 (s, 9H).

### Step 2: 5-(2-((tert-Butoxycarbonyl)(methyl)amino)ethoxy)-2-methylbenzoic acid (56A-2)

To a solution of methyl 5-(2-((tert-butoxycarbonyl)(methyl)amino)ethoxy)-2-methylbenzoate (550 mg, 1.70 mmol, 1.0 eq) in a mixture of MeOH (16 mL) and THF (8.0 mL) was added NaOH (2 M aqueous, 3.40 mL, 4.0 eq). The mixture was stirred at 70° C. for 16 h. LCMS indicated that the starting material was completely consumed, and the desired mass was detected. The reaction mixture was allowed to cool to room temperature, poured into H₂O (30 mL) and the mixture was washed with MTBE (15 mL×2). The aqueous layer was acidified to pH 5 using HCl (1 M aqueous). The product was extracted with EtOAc (8.0 mL×3). The combined organic layers were dried over Na₂SO₄, filtered, and concentrated under vacuum to give the crude product 5-(2-((tert-butoxycarbonyl)(methyl)amino)ethoxy)-2-methylbenzoic acid (470 mg), which was used in the next step without any further purification. M−56+H⁺=254.1 (LCMS); ¹H NMR (400 MHZ, CDCl₃) δ 7.58 (br d, J=1.9 Hz, 1H), 7.18 (d, J=8.4 Hz, 1H), 7.01 (dd, J=2.8, 8.4 Hz, 1H), 4.12 (br s, 2H), 3.62 (br s, 2H), 3.00 (s, 3H), 2.58 (s, 3H), 1.47 (s, 9H).

### Step 3: tert-Butyl methyl(2-(4-methyl-3-((1-(naphthalen-1-yl)cyclopropyl)carbamoyl) phenoxy)ethyl)carbamate (56A-3)

To a solution of 1-(naphthalen-1-yl)cyclopropanamine (90.6 mg, 495 μmol, 0.9 eq), 5-(2-((tert-butoxycarbonyl)(methyl)amino)ethoxy)-2-methylbenzoic acid (170 mg, 550 μmol, 1.0 eq) in DCM (6.0 mL) were added TEA (111 mg, 1.10 mmol, 153 μL, 2.0 eq), EDCI (126 mg, 659 μmol, 1.2 eq) and HOBt (89.1 mg, 659 μmol, 1.2 eq). The mixture was stirred at 20° C. for 16 h. LCMS indicated that the starting material was completely consumed, and the desired mass was detected. The reaction mixture was poured into H₂O (10 mL) and extracted with DCM (10 mL×2). The combined organic layers were dried over Na₂SO₄, filtered, and concentrated under vacuum to give a residue which was purified by flash silica gel chromatography using a gradient of EtOAc/petroleum ether from 1/100 to 1/1. tert-Butyl methyl(2-(4-methyl-3-((1-(naphthalen-1-yl)cyclopropyl)carbamoyl)phenoxy)ethyl)carbamate (90.0 mg, 190 μmol, 35% yield) was obtained as a yellow oil.

### Step 4: 2-Methyl-5-(2-(methylamino)ethoxy)-N-(1-(naphthalen-1-yl)cyclopropyl)benzamide (Compound 233)

To a solution of tert-butyl methyl(2-(4-methyl-3-((1-(naphthalen-1-yl)cyclopropyl)carbamoyl) phenoxy)ethyl)carbamate (90.0 mg, 190 μmol, 1.0 eq) in EtOAc (2.0 mL) was added HCl/EtOAc (4 M, 2.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 5%-55% B over 8 min; mobile phase A: 0.04% aqueous HCl, mobile phase B: acetonitrile). 2-Methyl-5-(2-(methylamino)ethoxy)-N-(1-(naphthalen-1-yl)cyclopropyl)benzamide (71.0 mg, 173 μmol, 91% yield, HCl salt) was obtained as a white solid. M+H⁺=375.1 (LCMS); ¹H NMR (400 MHZ, DMSO-d₆) δ 9.16 (s, 1H), 9.02 (br s, 1H), 8.66 (d, J=8.4 Hz, 1H), 7.93 (d, J=7.9 Hz, 1H), 7.86-7.78 (m, 2H), 7.62-7.39 (m, 3H), 7.07 (d, J=8.4 Hz, 1H), 6.89 (dd, J=2.6, 8.4 Hz, 1H), 6.67 (d, J=2.6 Hz, 1H), 4.16 (t, J=4.9 Hz, 2H), 3.29-3.17 (m, 2H), 2.56 (t, J=5.3 Hz, 3H), 1.96 (s, 3H), 1.36 (br s, 2H), 1.18 (br s, 2H).

### Example 57: 2-Methyl-5-((1-methylazetidin-2-yl)methoxy)-N—((S)-2,2,2-trifluoro-1-(naphthalen-1-yl)ethyl)benzamide (Compound 386)

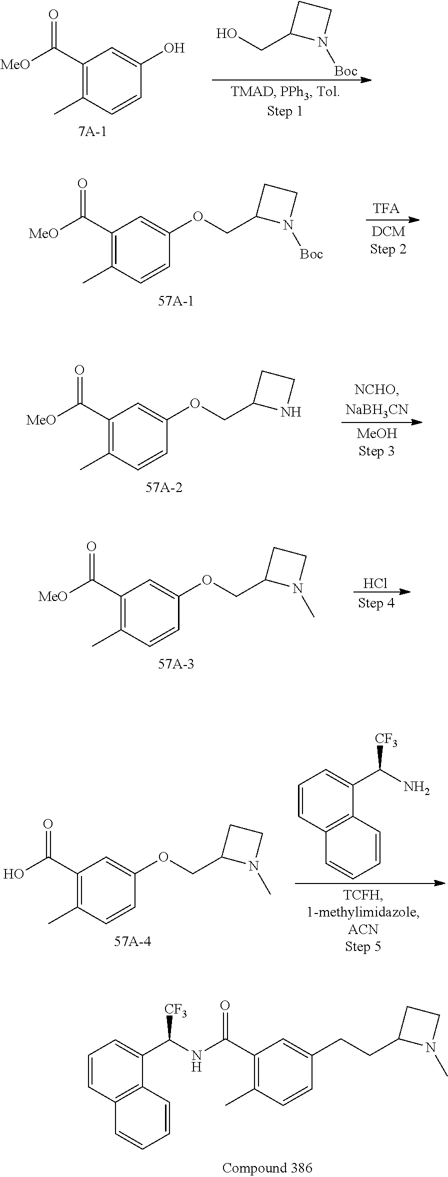

### Step 1: tert-Butyl 2-((3-(methoxycarbonyl)-4-methylphenoxy)methyl)azetidine-1-carboxylate (57A-1)

To a solution of methyl 5-hydroxy-2-methylbenzoate (500 mg, 3.01 mmol, 1.0 eq) and tert-butyl 2-(hydroxymethyl)azetidine-1-carboxylate (592 mg, 3.16 mmol, 1.1 eq) in toluene (15 mL) were added TMAD (1.55 g, 9.03 mmol, 3.0 eq) and PPh₃ (2.37 g, 9.03 mmol, 3.0 eq). The mixture was stirred at 100° C. for 16 h under a N₂ atmosphere. LCMS indicated that the starting material was completely consumed, and the desired mass was detected. The reaction mixture was allowed to cool to room temperature, poured into H₂O (30 mL) and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 0/1 to 1/4. tert-Butyl 2-((3-(methoxycarbonyl)-4-methylphenoxy)methyl)azetidine-1-carboxylate (900 mg, 2.68 mmol, 89% yield) was obtained as a yellow oil. M−56+H⁺=280.0 (LCMS); ¹H NMR (400 MHZ, CDCl₃) δ 7.50-7.47 (m, 1H), 7.17-7.12 (m, 1H), 7.03-6.97 (m, 1H), 4.56-4.45 (m, 1H), 4.34-4.24 (m, 1H), 4.13 (br d, J=4.0 Hz, 1H), 4.18-4.07 (m, 1H), 3.95 (br s, 4H), 2.55-2.51 (m, 3H), 2.42-2.20 (m, 2H), 1.47-1.40 (m, 9H).

### Step 2: Methyl 5-(azetidin-2-ylmethoxy)-2-methylbenzoate (57A-2)

To a solution of tert-butyl 2-((3-(methoxycarbonyl)-4-methylphenoxy)methyl)azetidine-1-carboxylate (800 mg, 2.39 mmol, 1.0 eq) in DCM (5.0 mL) was added TFA (3.26 g, 28.6 mmol, 2.12 mL, 12 eq). The mixture was stirred at 20° C. for 1 h. LCMS indicated that the starting material was completely consumed, and the desired mass was detected. The reaction mixture was concentrated under vacuum at 30° C. to give the crude product methyl 5-(azetidin-2-ylmethoxy)-2-methylbenzoate (800 mg, TFA salt) as a yellow oil, which was used in the next step without any further purification as a yellow oil. M+H⁺=236.1 (LCMS).

### Step 3: Methyl 2-methyl-5-((1-methylazetidin-2-yl)methoxy)benzoate (57A-3)

To a solution of (methyl 5-(azetidin-2-ylmethoxy)-2-methylbenzoate (400 mg, 1.03 mmol, 1.0 eq, TFA salt) in MeOH (8.0 mL) was added TEA (80.0 μL), followed by the addition of formaldehyde (335 mg, 4.12 mmol, 307 μL, 37% purity in water, 4.0 eq). The resulting mixture was adjusted to pH 6 with a small amount of AcOH. The mixture was stirred at 20° C. for 30 min, then NaBH₃CN (518 mg, 8.25 mmol, 8.0 eq) was added. The resulting reaction mixture was stirred at 20° C. for another 16 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give the crude product methyl 2-methyl-5-((1-methylazetidin-2-yl) methoxy)benzoate (400 mg) as a yellow oil, which was used in the next step without any further purification. M+H⁺=250.1 (LCMS).

### Step 4: 2-Methyl-5-((1-methylazetidin-2-yl)methoxy)benzoic acid (57A-4)

A solution of methyl 2-methyl-5-((1-methylazetidin-2-yl) methoxy)benzoate (300 mg, 1.20 mmol, 1.0 eq) in HCl (2 M aqueous 9.00 mL) was stirred at 100° C. for 16 h. LCMS indicated that the starting material was completely consumed, and the desired mass was detected. The reaction mixture was allowed to cool to room temperature, treated with H₂O (10 mL) and washed with MTBE (20 mL×2). The aqueous was acidified to pH 6 with NaOH (2 M aqueous). The product was extracted with DCM (10 mL×5) and the combined organic layers were dried over Na₂SO₄, filtered, and concentrated in vacuum to give the crude product 2-methyl-5-((1-methylazetidin-2-yl) methoxy)benzoic acid (220 mg) as a white solid, which was used in the next step without any further purification. M+H⁺=236.1 (LCMS).

### Step 5: 2-Methyl-5-((1-methylazetidin-2-yl)methoxy)-N—((S)-2,2,2-trifluoro-1-(naphthalen-1-yl)ethyl)benzamide (Compound 386)

To a solution of(S)-2,2,2-trifluoro-1-(naphthalen-1-yl)ethanamine (47.9 mg, 213 μmol, 1.0 eq) in acetonitrile (2.0 mL) was added 2-methyl-5-((1-methylazetidin-2-yl) methoxy)benzoic acid (50.0 mg, 213 μmol, 1.0 eq), followed by TCFH (71.6 mg, 255 μmol, 1.2 eq) and 1-methylimidazole (61.1 mg, 744 μmol, 59.3 μL, 3.5 eq). The mixture was stirred at 20° C. for 16 h. LCMS indicated that the starting material was completely consumed, and the desired mass was detected. The reaction mixture was poured into H₂O (5 mL) and extracted with EtOAc (5.0 mL×5). The combined organic layers were dried over Na₂SO₄, filtered, and concentrated under vacuum to give a residue which was purified by preparative HPLC (Phenomenex Gemini C18 column (80×30 mm, 10 μm); flow rate: 25 mL/min; gradient: 5%-20% B over 8 min; mobile phase A: 0.04% aqueous HCl, mobile phase B: acetonitrile). (S)-tert-Butyl 2-((3-(methoxycarbonyl)-4-methyl phenoxy)methyl)azetidine-1-carboxylate (46.1 mg, 93.6 μmol, 44% yield, HCl salt) was obtained as a yellow solid. M+H⁺=443.1 (LCMS); ¹H NMR (400 MHZ, DMSO-d₆) § 10.36-9.75 (m, 1H), 8.31-8.24 (m, 1H), 8.07-7.99 (m, 2H), 7.96-7.88 (m, 1H), 7.73-7.55 (m, 3H), 7.24-7.18 (m, 1H), 7.06-6.98 (m, 1H), 6.91-6.79 (m, 2H), 4.70-4.57 (m, 1H), 4.32-4.24 (m, 2H), 4.09-3.98 (m, 1H), 3.92-3.81 (m, 1H), 2.88-2.81 (m, 3H), 2.44-2.30 (m, 2H), 2.23-2.14 (m, 3H).

### Example 58: 5-(2-(Dimethylamino)ethoxy)-2-methyl-N-(1-(naphthalen-1-yl)cyclopropyl)benzamide (Compound 214)

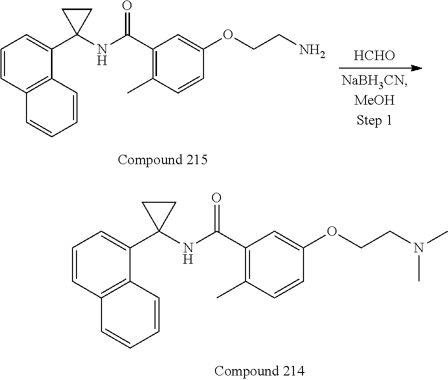

### Step 1: 5-(2-(Dimethylamino)ethoxy)-2-methyl-N-(1-(naphthalen-1-yl)cyclopropyl)benzamide (Compound 214)

To a solution of 5-(2-aminoethoxy)-2-methyl-N-(1-(naphthalen-1-yl)cyclopropyl)benzamide (130 mg, 361 μmol, 1.0 eq) in MeOH (3.0 mL) was added TEA (10.0 μL), followed by the addition of formaldehyde (58.5 mg, 721 μmol, 53.7 μL, 37% purity in water, 2.0 eq). The resulting mixture was adjusted to pH 6 with a small amount of AcOH. The mixture was stirred at 20° C. for 30 min, then NaBH₃CN (45.3 mg, 721 μmol, 2.0 eq) was added. The reaction mixture was stirred at 20° C. for another 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 5%-45% B over 8 min; mobile phase A: 0.04% aqueous HCl, mobile phase B: acetonitrile) to give 5-(2-(dimethylamino)ethoxy)-2-methyl-N-(1-(naphthalen-1-yl)cyclopropyl)benzamide (61.0 mg, 157 μmol, 43% yield, HCl salt) as a white solid. M+H⁺=389.1 (LCMS); ¹H NMR (400 MHZ, DMSO-d₆) δ 10.43 (br s, 1H), 9.14 (s, 1H), 8.66 (d, J=8.3 Hz, 1H), 7.93 (d, J=7.5 Hz, 1H), 7.85-7.80 (m, 2H), 7.61-7.43 (m, 3H), 7.07 (d, J=8.4 Hz, 1H), 6.90 (dd, J=2.8, 8.4 Hz, 1H), 6.69 (d, J=2.7 Hz, 1H), 4.26 (t, J=5.1 Hz, 2H), 3.45-3.41 (m, 2H), 2.78 (d, J=4.6 Hz, 6H), 1.96 (s, 3H), 1.39-1.33 (m, 2H), 1.20-1.15 (m, 2H).

### Example 59: 5-(2-(Ethylamino)ethoxy)-2-methyl-N-(1-(naphthalen-1-yl)cyclopropyl)benzamide (Compound 351)

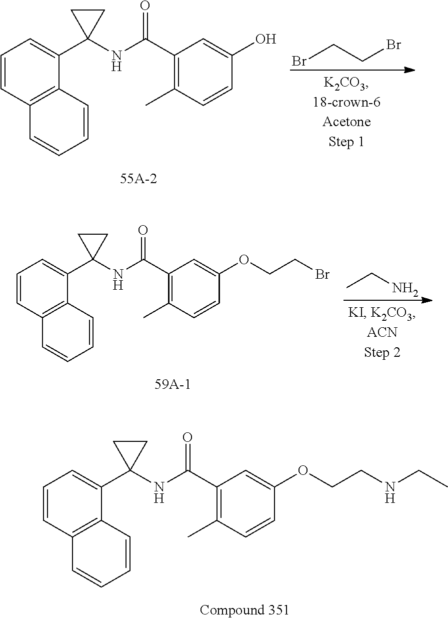

### Step 1: 5-(2-Bromoethoxy)-2-methyl-N-(1-(naphthalen-1-yl)cyclopropyl)benzamide (59A-1)

To a solution of 5-hydroxy-2-methyl-N-(1-(naphthalen-1-yl)cyclopropyl)benzamide (1.00 g, 3.15 mmol, 1.0 eq) and 1,2-dibromoethane (5.92 g, 31.5 mmol, 2.38 mL, 10 eq) in acetone (30 mL) were added potassium carbonate (1.00 g, 7.25 mmol, 2.3 eq) and 18-crown-6 (41.6 mg, 158 μmol, 0.05 eq). The mixture was stirred at 60° C. for 12 h. LCMS indicated that the starting material was completely consumed, and the desired mass was detected. The reaction mixture was allowed to cool to room temperature, poured into H₂O (30 mL) and extracted with EtOAc (10 mL×3). The combined organic layers were dried Na₂SO₄, filtered, and concentrated under vacuum to give a residue which was purified by flash silica gel chromatography using a gradient of EtOAc/petroleum ether from 1/100 to 100/1. 5-(2-Bromoethoxy)-2-methyl-N-(1-(naphthalen-1-yl)cyclopropyl)benzamide (800 mg, 1.89 mmol, 60% yield) was obtained as a colorless oil. M+H⁺=424.1 (LCMS); ¹H NMR (400 MHZ, DMSO-d₆) δ 9.09 (s, 1H), 8.66 (d, J=8.4 Hz, 1H), 7.93 (d, J=7.9 Hz, 1H), 7.86-7.79 (m, 2H), 7.61-7.42 (m, 3H), 7.04 (d, J=8.4 Hz, 1H), 6.86 (dd, J=2.8, 8.4 Hz, 1H), 6.64 (d, J=2.8 Hz, 1H), 4.26-4.20 (m, 2H), 3.77-3.72 (m, 2H), 1.96 (s, 3H), 1.39-1.34 (m, 2H), 1.19 (s, 2H).

### Step 2: 5-(2-(Ethylamino)ethoxy)-2-methyl-N-(1-(naphthalen-1-yl)cyclopropyl)benzamide (Compound 351)

To a solution of 5-(2-bromoethoxy)-2-methyl-N-(1-(naphthalen-1-yl)cyclopropyl)benzamide (70.0 mg, 165 μmol, 1.0 eq) in acetonitrile (3.0 mL) was added ethanamine (8.18 mg, 181 μmol, 11.9 μL, 1.1 eq), followed by KI (5.48 mg, 33.0 μmol, 0.2 eq) and K₂CO₃ (45.6 mg, 330 μmol, 2.0 eq). The mixture was stirred at 80° C. for 16 h. LCMS indicated that the starting material was completely consumed, and the desired mass was detected. The reaction mixture was allowed to cool to room temperature, poured into H₂O (5.0 mL) and extracted with EtOAc (6.0 mL×3). The combined organic layers were dried over Na₂SO₄, filtered, and concentrated under vacuum to give a residue which was purified by preparative HPLC (Phenomenex Gemini C18 column (75×30 mm, 3 μm); flow rate: 25 mL/min; gradient: 10%-40% B over 8 min; mobile phase A: 0.04% aqueous HCl, mobile phase B: acetonitrile). 5-(2-(Ethylamino)ethoxy)-2-methyl-N-(1-(naphthalen-1-yl)cyclopropyl)benzamide (33.0 mg, 77.7 μmol, 47% yield, HCl salt) was obtained as a yellow solid. M+H⁺=389.1 (LCMS); ¹H NMR (400 MHZ, DMSO-d₆) δ 9.14-9.11 (m, 1H), 8.67-8.61 (m, 1H), 7.96-7.90 (m, 1H), 7.85-7.79 (m, 2H), 7.61-7.43 (m, 3H), 7.10-7.04 (m, 1H), 6.93-6.85 (m, 1H), 6.68-6.64 (m, 1H), 4.18-4.10 (m, 2H), 3.29-3.21 (m, 2H), 3.02-2.92 (m, 2H), 1.99-1.93 (m, 3H), 1.38-1.32 (m, 2H), 1.22-1.14 (m, 5H).

### Example 60: 2-Methyl-N-(1-(naphthalen-1-yl)cyclopropyl)-5-(2-((2,2,2-trifluoroethyl)amino)ethoxy)benzamide (Compound 247)

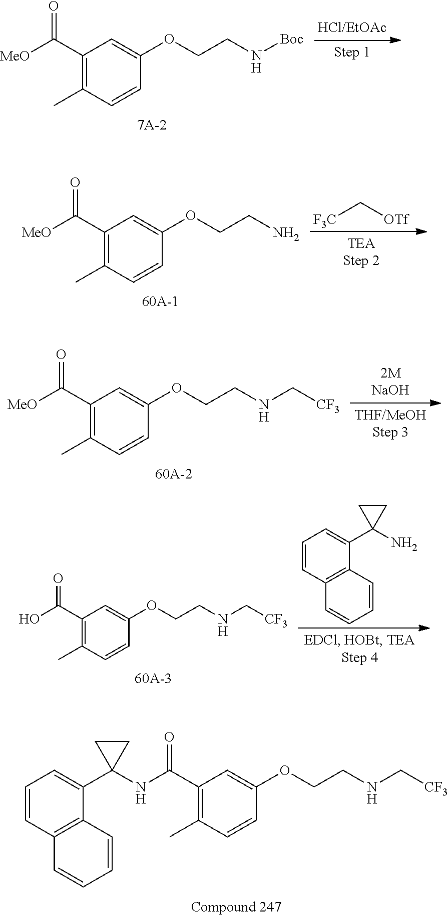

### Step 1: Methyl 5-(2-aminoethoxy)-2-methylbenzoate (60A-1)

To a solution of methyl 5-(2-((tert-butoxycarbonyl)amino)ethoxy)-2-methylbenzoate (500 mg, 1.62 mmol, 1.0 eq) in EtOAc (2.0 mL) was added HCl/EtOAc (4 M, 2.0 mL). The mixture was stirred at 20° C. for 1 h. TLC indicated that the starting material was completely consumed. The mixture was concentrated under vacuum to give the crude product methyl 5-(2-aminoethoxy)-2-methylbenzoate (400 mg, HCl salt), which was used in the next step without any further purification. M+H⁺=210.1 (LCMS).

### Step 2: Methyl 2-methyl-5-(2-((2,2,2-trifluoroethyl)amino)ethoxy)benzoate (60A-2)

To a solution of 2,2,2-trifluoroethyl trifluoromethanesulfonate (333 mg, 1.43 mmol, 1.0 eq) and methyl 5-(2-aminoethoxy)-2-methylbenzoate (300 mg, 1.43 mmol, 1.0 eq) in THF (6.0 mL) was added TEA (435 mg, 4.30 mmol, 599 μL, 3.0 eq). The mixture was stirred at 20° C. for 16 h. LCMS indicated that the starting material was completely consumed, and the desired mass was detected. The reaction mixture was poured into H₂O (6.0 mL) and extracted with EtOAc (3.0 mL×2). The combined organic layers were dried over Na₂SO₄, filtered, and concentrated under vacuum to give a residue which was purified by flash silica gel chromatography using a gradient of EtOAc/petroleum ether from 1/100 to 2/1. Methyl 2-methyl-5-(2-((2,2,2-trifluoroethyl)amino)ethoxy)benzoate (300 mg, 954 μmol, 67% yield) was obtained as a yellow oil. M+H⁺=292.0 (LCMS).

### Step 3: 2-Methyl-5-(2-((2,2,2-trifluoroethyl)amino)ethoxy)benzoic acid (60A-3)

To a solution of methyl 2-methyl-5-(2-((2,2,2-trifluoroethyl)amino)ethoxy)benzoate (300 mg, 1.03 mmol, 1.0 eq) in a mixture of MeOH (8.0 mL) and THF (4.0 mL) was added NaOH (2 M aqueous, 2.16 mL). The mixture was stirred at 20° C. for 16 h. LCMS indicated that 14% of the starting material remained and the 80% of desired mass was detected. The reaction mixture was poured into H₂O (6.0 mL) and washed with MTBE (3.0 mL×2). The aqueous layer was acidified to pH 5 with HCl (1 M aqueous). The product was extracted with EtOAc (2.0 mL×3). The combined organic layers were dried over Na₂SO₄, filtered, and concentrated under vacuum to give the crude product 2-methyl-5-(2-((2,2,2-trifluoroethyl)amino)ethoxy)benzoic acid (280 mg), which was used in the next step without any further purification. M+H⁺=278.0 (LCMS).

### Step 4: 2-Methyl-N-(1-(naphthalen-1-yl)cyclopropyl)-5-(2-((2,2,2-trifluoroethyl)amino)ethoxy)benzamide (Compound 247)

To a solution of 2-methyl-5-(2-((2,2,2-trifluoroethyl)amino)ethoxy)benzoic acid (80.0 mg, 289 μmol, 1.0 eq) and 1-(naphthalen-1-yl)cyclopropanamine (52.9 mg, 289 μmol, 1.0 eq) in DCM (5.0 mL) were added TEA (58.4 mg, 577 μmol, 80.3 μL, 2.0 eq), EDCI (66.4 mg, 346 μmol, 1.2 eq) and HOBt (46.8 mg, 346 μmol, 1.2 eq). The mixture was stirred at 20° C. for 2 h. LCMS indicated that the starting material was completely consumed, and the desired mass was detected. The reaction mixture was poured into H₂O (5.0 mL) and extracted with DCM (3.0 mL×2). The combined organic layers were dried over Na₂SO₄, filtered, and concentrated under vacuum to give a residue which was purified by preparative HPLC (Phenomenex C18 column (75×30 mm, 3 μm); flow rate: 25 mL/min; gradient: 40%-70% B over 8 min; mobile phase A: 0.04% aqueous HCl, mobile phase B: acetonitrile). 2-Methyl-N-(1-(naphthalen-1-yl)cyclopropyl)-5-(2-((2,2,2-trifluoroethyl)amino)ethoxy)benzamide (52.4 mg, 115 μmol, 40% yield, HCl salt) was obtained as a yellow oil. M+H⁺=443.1 (LCMS); ¹H NMR (400 MHz, CDCl₃) δ 8.46 (d, J=8.3 Hz, 1H), 7.98-7.86 (m, 2H), 7.81 (d, J=8.4 Hz, 1H), 7.63-7.43 (m, 3H), 7.01 (d, J=8.4 Hz, 1H), 6.77 (dd, J=2.6, 8.4 Hz, 1H), 6.69 (d, J=2.6 Hz, 1H), 6.68-6.66 (m, 1H), 3.95 (t, J=5.1 Hz, 2H), 3.23 (d, J=9.4 Hz, 2H), 3.05 (t, J=4.9 Hz, 2H), 2.11 (s, 3H), 1.43-1.37 (m, 2H), 1.27 (s, 2H).

### Example 61: 5-(2-(Isopropylamino)ethoxy)-2-methyl-N-(1-(naphthalen-1-yl)cyclopropyl)benzamide (Compound 362)

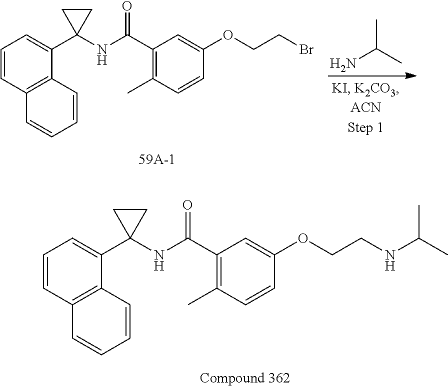

### Step 1: 5-(2-(Isopropylamino)ethoxy)-2-methyl-N-(1-(naphthalen-1-yl)cyclopropyl)benzamide (Compound 362)

To a solution of propan-2-amine (10.7 mg, 181 μmol, 15.6 μL, 1.1 eq) in ACN (5.0 mL) were added 5-(2-bromoethoxy)-2-methyl-N-(1-(naphthalen-1-yl)cyclopropyl)benzamide (70.0 mg, 165 μmol, 1.0 eq), KI (2.74 mg, 16.5 μmol, 0.1 eq) and K₂CO₃ (45.6 mg, 330 μmol, 2.0 eq). The mixture was stirred at 80° C. for 16 h. LCMS indicated that the starting material was completely consumed, and the desired mass was detected. The reaction mixture was allowed to cool to room temperature, poured into H₂O (4.0 mL) and extracted with EtOAc (3.0 mL×3). The combined organic layers were dried over Na₂SO₄, filtered, and concentrated under vacuum to give a residue which was purified by preparative HPLC (Phenomenex Luna (80×30 mm, 3 μm); flow rate: 25 mL/min; gradient: 5%-50% B over 8 min; mobile phase A: 0.04% aqueous HCl, mobile phase B: acetonitrile). 5-(2-(Isopropylamino)ethoxy)-2-methyl-N-(1-(naphthalen-1-yl)cyclopropyl)benzamide (21.9 mg, 49.6 μmol, 30% yield, HCl salt) was obtained as a white solid. M+H⁺=403.1 (LCMS); ¹H NMR (400 MHZ, DMSO-d₆) δ 9.18-9.08 (m, 1H), 8.74-8.69 (m, 1H), 8.68-8.63 (m, 1H), 7.97-7.91 (m, 1H), 7.86-7.80 (m, 2H), 7.61-7.43 (m, 3H), 7.12-7.05 (m, 1H), 6.93-6.86 (m, 1H), 6.70-6.65 (m, 1H), 4.19-4.13 (m, 2H), 3.26 (br s, 3H), 1.97 (s, 3H), 1.40-1.33 (m, 2H), 1.23 (d, J=6.5 Hz, 6H), 1.20-1.16 (m, 2H).

### Example 62: 5-(2-((Cyclopropylmethyl)amino)ethoxy)-2-methyl-N-(1-(naphthalen-1-yl)cyclopropyl)benzamide (Compound 361)

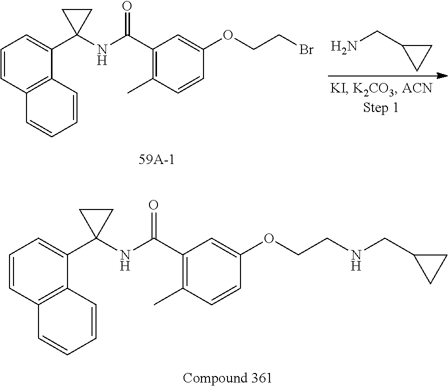 [SMILES: Cc1ccc(OCCBr)cc1C(=O)NC1(c2cccc3ccccc23)CC1 ; NCC1CC1 ; Cc1ccc(OCCNCC2CC2)cc1C(=O)NC1(c2cccc3ccccc23)CC1]

### Step 1: 5-(2-((Cyclopropylmethyl)amino)ethoxy)-2-methyl-N-(1-(naphthalen-1-yl)cyclopropyl)benzamide (Compound 361)

To a solution of cyclopropylmethanamine (12.9 mg, 181 μmol, 1.1 eq) in ACN (5.0 mL) were added 5-(2-bromoethoxy)-2-methyl-N-(1-(naphthalen-1-yl)cyclopropyl)benzamide (70.0 mg, 165 μmol, 1.0 eq), KI (2.74 mg, 16.5 μmol, 0.1 eq) and K₂CO₃ (45.6 mg, 330 μmol, 2.0 eq). The mixture was stirred at 80° C. for 16 h. LCMS indicated that the starting material was completely consumed, and the desired mass was detected. The reaction mixture was allowed to cool to room temperature, poured into H₂O (4.0 mL) and extracted with EtOAc (3.0 mL×3). The combined organic layers were dried over Na₂SO₄, filtered, and concentrated under vacuum to give a residue which was purified by preparative HPLC (Phenomenex Luna (80×30 mm, 3 μm); flow rate: 25 mL/min; gradient: 5%-50% B over 8 min; mobile phase A: 0.04% aqueous HCl, mobile phase B: acetonitrile). 5-(2-((Cyclopropylmethyl)amino)ethoxy)-2-methyl-N-(1-(naphthalen-1-yl)cyclopropyl)benzamide (18.9 mg, 41.6 μmol, 25% yield, HCl salt) was obtained as a white solid. M+H⁺=415.1 (LCMS); ¹H NMR (400 MHZ, DMSO-d₆) δ 9.13 (s, 1H), 9.01-8.80 (m, 2H), 8.72-8.61 (m, 1H), 7.98-7.91 (m, 1H), 7.82 (br d, J=5.9 Hz, 2H), 7.62-7.42 (m, 3H), 7.13-7.03 (m, 1H), 6.95-6.86 (m, 1H), 6.72-6.63 (m, 1H), 4.23-4.12 (m, 2H), 3.29 (br d, J=4.9 Hz, 2H), 2.93-2.79 (m, 2H), 1.97 (s, 3H), 1.45-1.30 (m, 2H), 1.24-1.14 (m, 2H), 1.11-0.99 (m, 1H), 0.63-0.50 (m, 2H), 0.35 (br s, 2H).

### Example 63: 2-Methyl-N-(1-(naphthalen-1-yl)cyclopropyl)-5-(2-(phenylamino)ethoxy)benzamide (Compound 315)

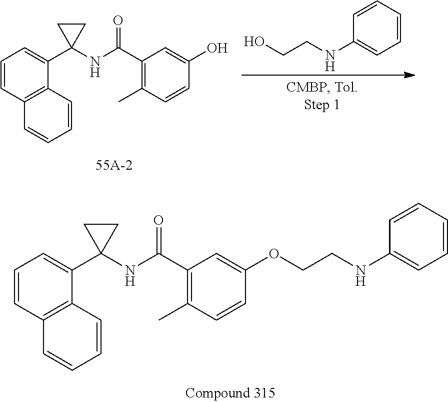 [SMILES: Cc1ccc(O)cc1C(=O)NC1(c2cccc3ccccc23)CC1 ; OCCNc1ccccc1 ; Cc1ccc(OCCNc2ccccc2)cc1C(=O)NC1(c2cccc3ccccc23)CC1]

### Step 1: 2-Methyl-N-(1-(naphthalen-1-yl)cyclopropyl)-5-(2-(phenylamino)ethoxy)benzamide (Compound 315)

A mixture of 5-hydroxy-2-methyl-N-(1-(naphthalen-1-yl)cyclopropyl)benzamide (100 mg, 315 μmol, 1.0 eq) and 2-(phenylamino) ethanol (43.2 mg, 315 μmol, 1.0 eq) in anhydrous toluene (5.0 mL) was degassed and purged with N₂ three times. To the mixture was added CMBP (114 mg, 473 μmol, 1.5 eq) dropwise at 20° C. The mixture was stirred at 110° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 30%-55% B over 8 min; mobile phase A: 0.04% aqueous HCl, mobile phase B: acetonitrile). 2-Methyl-N-(1-(naphthalen-1-yl)cyclopropyl)-5-(2-(phenylamino)ethoxy)benzamide (55.9 mg, 113 μmol, 95% yield, HCl salt) was obtained as a white solid. M+H⁺=437.2 (LCMS); ¹H NMR (400 MHZ, DMSO-d₆) δ 9.09 (s, 1H), 8.65 (d, J=8.3 Hz, 1H), 7.96-7.90 (m, 1H), 7.85-7.78 (m, 2H), 7.58-7.42 (m, 3H), 7.13 (t, J=7.8 Hz, 2H), 7.03 (d, J=8.5 Hz, 1H), 6.85 (dd, J=2.6, 8.4 Hz, 1H), 6.77-6.60 (m, 4H), 4.02 (t, J=5.5 Hz, 2H), 3.41 (br s, 2H), 1.96 (s, 3H), 1.39-1.32 (m, 2H), 1.20-1.13 (m, 2H).

### Example 64: 5-(2-(Benzylamino)ethoxy)-2-methyl-N-(1-(naphthalen-1-yl)cyclopropyl)benzamide (Compound 360)

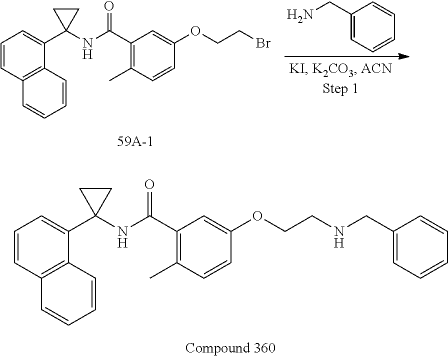 [SMILES: Cc1ccc(OCCBr)cc1C(=O)NC1(c2cccc3ccccc23)CC1 ; NCc1ccccc1 ; Cc1ccc(OCCNCc2ccccc2)cc1C(=O)NC1(c2cccc3ccccc23)CC1]

### Step 1: 5-(2-(Benzylamino)ethoxy)-2-methyl-N-(1-(naphthalen-1-yl)cyclopropyl)benzamide (Compound 360)

To a solution of 5-(2-bromoethoxy)-2-methyl-N-(1-(naphthalen-1-yl)cyclopropyl)benzamide (50.0 mg, 118 μmol, 1.0 eq) and phenylmethanamine (13.9 mg, 130 μmol, 14.1 mL, 1.1 eq) in acetonitrile (5.0 mL) were added potassium iodide (1.96 mg, 11.8 μmol, 0.1 eq) and potassium carbonate (32.6 mg, 236 μmol, 2.0 eq). The mixture was stirred at 80° C. for 16 h. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 10%-40% B over 8 min; mobile phase A: 0.04% aqueous HCl, mobile phase B: acetonitrile). 5-(2-(Benzylamino)ethoxy)-2-methyl-N-(1-(naphthalen-1-yl)cyclopropyl)benzamide (64.6 mg, 132 μmol, 28% yield, HCl salt) was obtained as a white solid. M+H⁺=451.1 (LCMS); ¹H NMR (400 MHZ, DMSO-d₆) δ 9.26 (br s, 2H), 9.12 (s, 1H), 8.65 (d, J=8.3 Hz, 1H), 7.93 (d, J=7.4 Hz, 1H), 7.87-7.79 (m, 2H), 7.61-7.38 (m, 8H), 7.07 (d, J=8.4 Hz, 1H), 6.88 (dd, J=2.7, 8.4 Hz, 1H), 6.66 (d, J=2.8 Hz, 1H), 4.26-4.10 (m, 4H), 3.25 (br s, 2H), 1.97 (s, 3H), 1.40-1.31 (m, 2H), 1.20-1.15 (m, 2H).

### Example 65: 5-(2-((2-Hydroxyethyl)amino)ethoxy)-2-methyl-N-(1-(naphthalen-1-yl)cyclopropyl)benzamide (Compound 359)

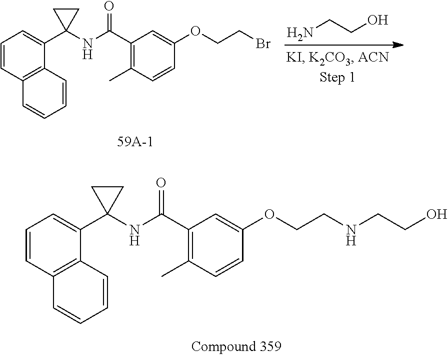

### Step 1: 5-(2-((2-Hydroxyethyl)amino)ethoxy)-2-methyl-N-(1-(naphthalen-1-yl)cyclopropyl)benzamide (Compound 359)

To a solution of 5-(2-bromoethoxy)-2-methyl-N-(1-(naphthalen-1-yl)cyclopropyl)benzamide (70.0 mg, 165 μmol, 1.0 eq) and 2-aminoethanol (11.1 mg, 181 μmol, 11.0 mL, 1.1 eq) in acetonitrile (5.0 mL) were added potassium iodide (2.74 mg, 16.5 μmol, 0.1 eq) and potassium carbonate (45.6 mg, 330 μmol, 2.0 eq). The mixture was stirred at 80° C. for 16 h. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 1%-40% B over 8 min; mobile phase A: 0.04% aqueous HCl, mobile phase B: acetonitrile). 5-(2-((2-Hydroxyethyl)amino)ethoxy)-2-methyl-N-(1-(naphthalen-1-yl)cyclopropyl)benzamide (16.1 mg, 36.5 μmol, 22% yield, HCl salt) was obtained as a white solid. M+H⁺=405.1 (LCMS); ¹H NMR (400 MHZ, DMSO-d₆) δ 9.12 (s, 1H), 8.72 (br d, J=2.9 Hz, 2H), 8.65 (d, J=8.4 Hz, 1H), 7.93 (d, J=8.0 Hz, 1H), 7.83 (t, J=6.5 Hz, 2H), 7.61-7.43 (m, 3H), 7.08 (d, J=8.5 Hz, 1H), 6.88 (dd, J=2.6, 8.4 Hz, 1H), 6.67 (d, J=2.8 Hz, 1H), 5.31-5.13 (m, 1H), 4.17 (t, J=5.1 Hz, 2H), 3.65 (t, J=5.3 Hz, 2H), 3.35-3.25 (m, 2H), 3.09-2.99 (m, 2H), 1.97 (s, 3H), 1.40-1.31 (m, 2H), 1.21-1.16 (m, 2H).

### Example 66: 5-(2-((2-Methoxyethyl)amino)ethoxy)-2-methyl-N-(1-(naphthalen-1-yl)cyclopropyl)benzamide (Compound 358)

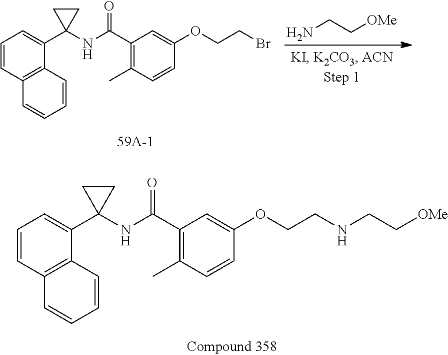 [SMILES: Cc1ccc(OCCBr)cc1C(=O)NC1(c2cccc3ccccc23)CC1 ; COCCN ; COCCNCCOc1ccc(C)c(C(=O)NC2(c3cccc4ccccc34)CC2)c1]

### Step 1: 5-(2-((2-Methoxyethyl)amino)ethoxy)-2-methyl-N-(1-(naphthalen-1-yl)cyclopropyl)benzamide (Compound 358)

To a solution of 5-(2-bromoethoxy)-2-methyl-N-(1-(naphthalen-1-yl)cyclopropyl)benzamide (50.0 mg, 118 μmol, 1.0 eq) and 2-methoxyethanamine (9.74 mg, 130 μmol, 11.3 mL, 1.1 eq) in acetonitrile (5.0 mL) were added potassium iodide (1.96 mg, 11.8 μmol, 0.1 eq) and potassium carbonate (32.6 mg, 236 μmol, 2.0 eq). The mixture was stirred at 80° C. for 16 h. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 1%-40% B over 8 min; mobile phase A: 0.04% aqueous HCl, mobile phase B: acetonitrile). 5-(2-((2-Methoxyethyl)amino)ethoxy)-2-methyl-N-(1-(naphthalen-1-yl)cyclopropyl)benzamide (19.0 mg, 41.6 μmol, 18% yield, HCl salt) was obtained as a yellow solid. M+H⁺=419.1 (LCMS); ¹H NMR (400 MHZ, DMSO-d₆) δ 9.12 (s, 1H), 8.79 (br d, J=1.4 Hz, 2H), 8.65 (d, J=8.1 Hz, 1H), 7.94 (d, J=7.5 Hz, 1H), 7.87-7.80 (m, 2H), 7.61-7.43 (m, 3H), 7.08 (d, J=8.4 Hz, 1H), 6.88 (dd, J=2.7, 8.3 Hz, 1H), 6.66 (d, J=2.6 Hz, 1H), 4.16 (br t, J=5.1 Hz, 2H), 3.58 (t, J=5.1 Hz, 2H), 3.28 (s, 5H), 3.16 (quin, J=5.3 Hz, 2H), 1.97 (s, 3H), 1.35 (s, 2H), 1.21-1.15 (m, 2H).

### Example 67: 5-(2-(3-Fluoroazetidin-1-yl)ethoxy)-2-methyl-N-(1-(naphthalen-1-yl)cyclopropyl)benzamide (Compound 300)

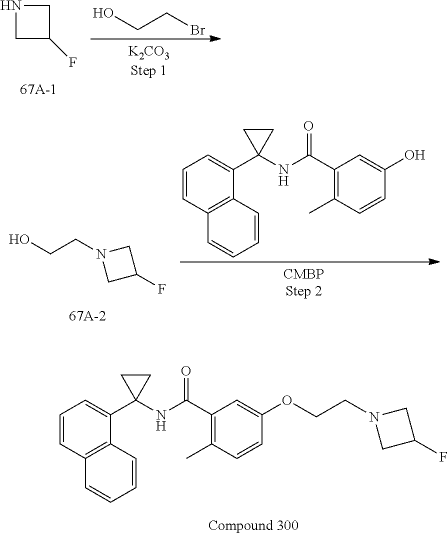

### Step 1: 2-(3-Fluoroazetidin-1-yl)ethanol (67A-2)

To a solution of 3-fluoroazetidine (300 mg, 2.69 mmol, 1.0 eq, HCl salt) in acetonitrile (6.0 mL) was added K₂CO₃ (1.12 g, 8.07 mmol, 3.0 eq). The mixture was stirred at 20° C. for 30 min, then 2-bromoethanol (336 mg, 2.69 mmol, 191 μL, 1.0 eq) was added. The mixture was stirred at 80° C. for 16 h. TLC indicated that the starting material was completely consumed. The reaction mixture was allowed to cool to room temperature, poured into H₂O (6.0 mL) and extracted with EtOAc (2.0 mL×3). The combined organic layers were dried over Na₂SO₄, filtered, and concentrated under vacuum to give a residue which was purified by preparative TLC (DCM/MeOH=10/1, R_{f}=0.5). 2-(3-Fluoroazetidin-1-yl)ethanol (80.0 mg, 672 μmol, 25% yield) was obtained as a yellow oil. ¹H NMR (400 MHZ, CDCl₃) δ 3.89-3.83 (m, 1H), 3.75 (dd, J=1.7, 5.2 Hz, 1H), 3.72-3.67 (m, 4H), 3.66-3.59 (m, 2H), 2.84-2.73 (m, 2H).

### Step 2: 5-(2-(3-Fluoroazetidin-1-yl)ethoxy)-2-methyl-N-(1-(naphthalen-1-yl)cyclopropyl)benzamide (Compound 300)

To a solution of 2-(3-fluoroazetidin-1-yl)ethanol (18.8 mg, 158 μmol, 1.0 eq) and 5-hydroxy-2-methyl-N-(1-(naphthalen-1-yl)cyclopropyl)benzamide (50.0 mg, 158 μmol, 1.0 eq) in toluene (2.0 mL) was added CMBP (38.0 mg, 158 μmol, 1.0 eq). The resulting mixture was degassed and purged with N₂ three times and then the mixture was stirred at 110° C. for 16 h under a N₂ atmosphere. LCMS indicated that the starting material was completely consumed, and the desired mass was detected. The reaction mixture was allowed to cool to room temperature, poured into H₂O (2.0 mL) and extracted with EtOAc (1.0 mL×2). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 20%-60% B over 8 min; mobile phase A: 0.2% aqueous FA, mobile phase B: acetonitrile). 5-(2-(3-Fluoroazetidin-1-yl)ethoxy)-2-methyl-N-(1-(naphthalen-1-yl)cyclopropyl)benzamide (8.20 mg, 19.6 μmol, 12% yield, FA salt) was obtained as a white solid. M+H⁺=419.1 (LCMS); ¹H NMR (400 MHZ, DMSO-d₆) δ 9.08 (s, 1H), 8.65 (d, J=8.3 Hz, 1H), 7.93 (d, J=7.9 Hz, 1H), 7.81 (dd, J=7.9, 10.0 Hz, 2H), 7.60-7.41 (m, 3H), 7.02 (d, J=8.4 Hz, 1H), 6.80 (dd, J=2.7, 8.3 Hz, 1H), 6.58 (d, J=2.6 Hz, 1H), 5.27-4.97 (m, 1H), 3.84 (t, J=5.4 Hz, 2H), 3.62-3.49 (m, 2H), 3.20-3.05 (m, 2H), 2.73 (t, J=5.4 Hz, 2H), 1.95 (s, 3H), 1.35 (s, 2H), 1.19-1.12 (m, 2H).

### Example 68: 2-Methyl-N-(1-(naphthalen-1-yl)cyclopropyl)-5-(2-(piperidin-1-yl)ethoxy)benzamide (Compound 254)

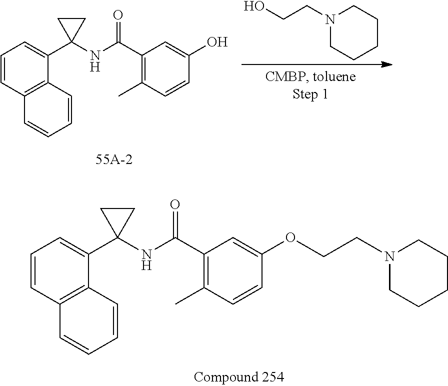 [SMILES: Cc1ccc(O)cc1C(=O)NC1(c2cccc3ccccc23)CC1 ; OCCN1CCCCC1 ; Cc1ccc(OCCN2CCCCC2)cc1C(=O)NC1(c2cccc3ccccc23)CC1]

### Step 1: 2-Methyl-N-(1-(naphthalen-1-yl)cyclopropyl)-5-(2-(piperidin-1-yl)ethoxy)benzamide (Compound 254)

A mixture of 5-hydroxy-2-methyl-N-(1-(naphthalen-1-yl)cyclopropyl)benzamide (70.0 mg, 221 μmol, 1.0 eq), 2-(piperidin-1-yl)ethanol (28.5 mg, 221 μmol, 29.3 μL, 1.0 eq) and CMBP (79.9 mg, 331 μmol, 1.5 eq) in toluene (3.5 mL) was degassed and purged with N₂ three times. The mixture was stirred at 110° C. for 16 h under a N₂ atmosphere. LCMS indicated that the starting material was completely consumed, and the desired mass was detected. The reaction mixture was poured into H₂O (3.0 mL) and extracted with EtOAc (3.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 15%-45% B over 8 min; mobile phase A: 0.2% aqueous FA, mobile phase B: acetonitrile). 2-Methyl-N-(1-(naphthalen-1-yl)cyclopropyl)-5-(2-(piperidin-1-yl)ethoxy)benzamide (60.1 mg, 140 μmol, 64% yield) was obtained as a yellow solid. M+H⁺=429.1 (LCMS); ¹H NMR (400 MHZ, DMSO-d₆) δ 9.09 (s, 1H), 8.66 (d, J=8.1 Hz, 1H), 7.93 (d, J=7.6 Hz, 1H), 7.86-7.77 (m, 2H), 7.61-7.42 (m, 3H), 7.02 (d, J=8.6 Hz, 1H), 6.83 (dd, J=2.7, 8.4 Hz, 1H), 6.61 (d, J=2.7 Hz, 1H), 3.96 (t, J=5.9 Hz, 2H), 2.65-2.57 (m, 3H), 2.39 (br s, 3H), 1.95 (s, 3H), 1.47 (quin, J=5.5 Hz, 4H), 1.40-1.29 (m, 4H), 1.22-1.11 (m, 2H).

### Example 69: 5-(2-(4,4-Difluoropiperidin-1-yl)ethoxy)-2-methyl-N-(1-(naphthalen-1-yl)cyclopropyl)benzamide (Compound 284)

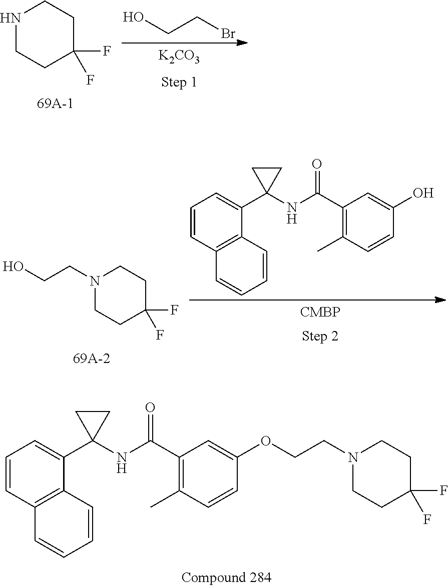

### Step 1: 2-(4,4-Difluoropiperidin-1-yl)ethanol (69A-2)

To a solution of 4,4-difluoropiperidine (500 mg, 4.13 mmol, 1.0 eq) in ACN (20 mL) were added K₂CO₃ (1.71 g, 12.4 mmol, 3.0 eq) and 2-bromoethanol (2.58 g, 20.6 mmol, 1.47 mL, 5.0 eq). The mixture was stirred at 90° C. for 16 h. TLC indicated that the starting material was completely consumed. The reaction mixture was allowed to cool to room temperature, poured into H₂O (20 mL) and extracted with EtOAc (7.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/0. 2-(4,4-Difluoropiperidin-1-yl)ethanol (330 mg, 2.00 mmol, 48% yield) was obtained as a yellow oil. ¹H NMR (400 MHZ, CDCl₃) δ 3.62 (t, J=5.4 Hz, 2H), 2.68-2.54 (m, 6H), 2.16-1.85 (m, 4H).

### Step 2: 5-(2-(4,4-Difluoropiperidin-1-yl)ethoxy)-2-methyl-N-(1-(naphthalen-1-yl)cyclopropyl)benzamide (Compound 284)

To a solution of 2-(4,4-difluoropiperidin-1-yl)ethanol (52.0 mg, 315 μmol, 1.0 eq), 5-hydroxy-2-methyl-N-(1-(naphthalen-1-yl)cyclopropyl)benzamide (100 mg, 315 μmol, 1.0 eq) in toluene (2.5 mL) was added CMBP (76.1 mg, 315 μmol, 1.0 eq). The mixture was stirred at 110° C. for 16 h under a N₂ atmosphere. LCMS indicated that the starting material was completely consumed, and the desired mass was detected. The reaction mixture was allowed to cool to room temperature, poured into H₂O (3.0 mL) and extracted with EtOAc (1.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 1%-50% B over 8 min; mobile phase A: 0.2% aqueous FA, mobile phase B: acetonitrile). 5-(2-(4,4-Difluoropiperidin-1-yl)ethoxy)-2-methyl-N-(1-(naphthalen-1-yl)cyclopropyl)benzamide (67.8 mg, 143 μmol, 45% yield, FA salt) was obtained as a white solid. M+H⁺=465.1 (LCMS); ¹H NMR (400 MHZ, DMSO-d₆) δ 9.08 (s, 1H), 8.66 (d, J=8.4 Hz, 1H), 7.96-7.91 (m, 1H), 7.87-7.78 (m, 2H), 7.61-7.41 (m, 3H), 7.03 (d, J=8.5 Hz, 1H), 6.83 (dd, J=2.7, 8.3 Hz, 1H), 6.62 (d, J=2.8 Hz, 1H), 3.98 (t, J=5.7 Hz, 2H), 2.72 (t, J=5.7 Hz, 2H), 2.57 (br t, J=5.4 Hz, 4H), 1.96 (s, 7H), 1.36 (br d, J=1.6 Hz, 2H), 1.23-1.12 (m, 2H).

### Example 70: 2-Methyl-5-(2-morpholinoethoxy)-N-(1-(naphthalen-1-yl)cyclopropyl)benzamide (Compound 209)

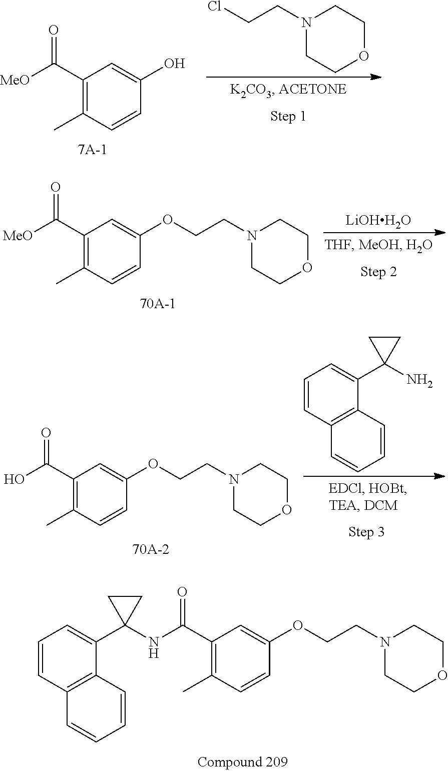

### Step 1: Methyl 2-methyl-5-(2-morpholinoethoxy)benzoate (70A-1)

To a solution of methyl 5-hydroxy-2-methylbenzoate (500 mg, 3.01 mmol, 1.0 eq) and 4-(2-chloroethyl) morpholine (804 mg, 3.61 mmol, 1.2 eq) in acetone (20 mL) was added K₂CO₃ (1.66 g, 12.0 mmol, 4.0 eq). The mixture was stirred at 60° C. for 16 h. LCMS indicated that the starting material was completely consumed, and the desired mass was detected. The reaction mixture was allowed to cool to room temperature, poured into H₂O (20 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/0. Methyl 2-methyl-5-(2-morpholinoethoxy)benzoate (300 mg, 1.07 mmol, 36% yield) was obtained as a colorless oil. M+H⁺=280.2 (LCMS); ¹H NMR (400 MHZ, CDCl₃) δ 7.46 (d, J=2.8 Hz, 1H), 7.14 (d, J=8.4 Hz, 1H), 6.97 (dd, J=2.8, 8.4 Hz, 1H), 4.12 (t, J=5.7 Hz, 2H), 3.89 (s, 3H), 3.77-3.71 (m, 4H), 2.80 (t, J=5.7 Hz, 2H), 2.61-2.55 (m, 4H), 2.51 (s, 3H).

### Step 2: 2-Methyl-5-(2-morpholinoethoxy)benzoic acid (70A-2)

To a solution of methyl 2-methyl-5-(2-morpholinoethoxy)benzoate (200 mg, 716 μmol, 1.0 eq) in a mixture of MeOH (4.0 mL), H₂O (2.0 mL) and THF (8.0 mL) was added LiOH·H₂O (60.1 mg, 1.43 mmol, 2.0 eq). The mixture was stirred at 25° C. for 1 h, then the mixture was stirred at 70° C. for 2 h. TLC indicated that the starting material was completely consumed. The reaction mixture was allowed to cool to room temperature, poured into H₂O (10 mL) and washed with MTBE (5.0 mL×3). The aqueous layer was acidified to pH 5 using HCl (1 M aqueous). The product was extracted with dimethyltetrahydrofuran (5.0 mL×3). The combined organic layers were dried over Na₂SO₄, filtered, and concentrated under vacuum to give the crude product 2-methyl-5-(2-morpholinoethoxy)benzoic acid (80.0 mg), which was used in the next step without any further purification. ¹H NMR (400 MHZ, DMSO-d₆) δ 7.33 (d, J=2.6 Hz, 1H), 7.19 (d, J=8.4 Hz, 1H), 7.04 (dd, J=2.6, 8.4 Hz, 1H), 4.09 (br t, J=5.6 Hz, 2H), 3.60-3.56 (m, 4H), 2.71 (br s, 2H), 2.54-2.51 (m, 4H), 2.42 (s, 3H).

### Step 3: 2-Methyl-5-(2-morpholinoethoxy)-N-(1-(naphthalen-1-yl)cyclopropyl)benzamide (Compound 209)

To a solution of 1-(naphthalen-1-yl)cyclopropanamine (50.0 mg, 273 μmol, 1.0 eq) and 2-methyl-5-(2-morpholinoethoxy)benzoic acid (72.4 mg, 273 μmol, 1.0 eq) in DCM (10 mL) were added TEA (82.8 mg, 819 μmol, 114 μL, 3.0 eq), EDCI (105 mg, 546 μmol, 3.0 eq) and HOBt (73.7 mg, 546 μmol, 2.0 eq). The mixture was stirred at 20° C. for 2 h. LCMS indicated that the starting material was completely consumed, and the desired mass was detected. The reaction mixture was poured into H₂O (10 mL) and extracted with DCM (3.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 35%-65% B over 8 min; mobile phase A: 10 mM aqueous NH₄HCO₃, mobile phase B: acetonitrile). 2-Methyl-5-(2-morpholinoethoxy)-N-(1-(naphthalen-1-yl)cyclopropyl)benzamide (45.4 mg, 102 μmol, 38% yield) was obtained as a white solid. M+H⁺=431.1 (LCMS); ¹H NMR (400 MHZ, CDCl₃) δ 8.45 (d, J=8.4 Hz, 1H), 7.92 (dd, J=7.8, 15.8 Hz, 2H), 7.81 (d, J=8.3 Hz, 1H), 7.61-7.55 (m, 1H), 7.54-7.45 (m, 2H), 7.00 (d, J=8.4 Hz, 1H), 6.78 (dd, J=2.7, 8.4 Hz, 1H), 6.70 (d, J=2.7 Hz, 1H), 3.99 (t, J=5.6 Hz, 2H), 3.76-3.67 (m, 4H), 2.72 (t, J=5.6 Hz, 2H), 2.56-2.49 (m, 4H), 2.11 (s, 3H), 1.57 (br s, 2H), 1.42-1.37 (m, 2H).

### Example 71: 5-(2-Aminopropoxy)-2-methyl-N-(1-(naphthalen-1-yl)cyclopropyl)benzamide (Compound 324)

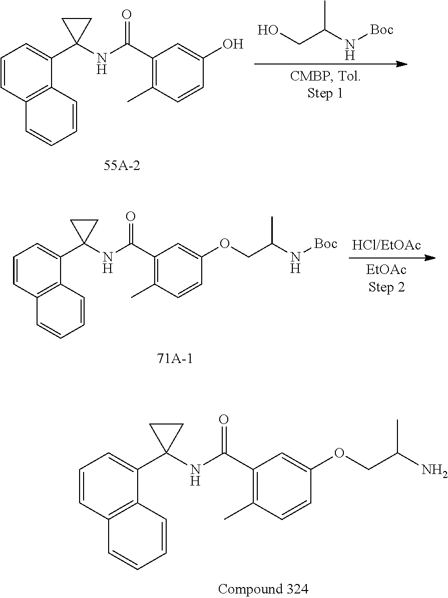 [SMILES: Cc1ccc(O)cc1C(=O)NC1(c2cccc3ccccc23)CC1 ; CC(CO)NC(=O)OC(C)(C)C ; Cc1ccc(OCC(C)NC(=O)OC(C)(C)C)cc1C(=O)NC1(c2cccc3ccccc23)CC1 ; Cc1ccc(OCC(C)N)cc1C(=O)NC1(c2cccc3ccccc23)CC1]

### Step 1: tert-Butyl(1-(4-methyl-3-((1-(naphthalen-1-yl)cyclopropyl)carbamoyl)phenoxy)propan-2-yl)carbamate (71A-1)

A mixture of 5-hydroxy-2-methyl-N-(1-(naphthalen-1-yl)cyclopropyl)benzamide (100 mg, 315 μmol, 1.0 eq), tert-butyl(1-hydroxypropan-2-yl)carbamate (55.2 mg, 315 μmol, 1.0 eq) and CMBP (114 mg, 473 μmol, 1.5 eq) in toluene (5.0 mL) was degassed and purged with N₂ three times. The resulting mixture was stirred at 110° C. for 16 h under a N₂ atmosphere. LCMS indicated that the starting material was completely consumed, and the desired mass was detected. The reaction mixture was allowed to cool to room temperature, poured into H₂O (4.0 mL) and extracted with DCM (3.0 mL×3). The combined organic layers were dried over Na₂SO₄, filtered, and concentrated under vacuum to give a residue which was used without further purification. tert-Butyl(1-(4-methyl-3-((1-(naphthalene-1-yl)cyclopropyl)carbamoyl)phenoxy) propan-2-yl)carbamate (90.0 mg, 190 μmol, 60% yield) was obtained as a white solid.

### Step 2: 5-(2-Aminopropoxy)-2-methyl-N-(1-(naphthalen-1-yl)cyclopropyl)benzamide (Compound 324)

To a stirred solution of tert-butyl(1-(4-methyl-3-((1-(naphthalen-1-yl)cyclopropyl)carbamoyl) phenoxy) propan-2-yl)carbamate (80.0 mg, 169 μmol, 1.0 eq) in EtOAc (8.0 mL) was added HCl/EtOAc (4 M, 8.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 25%-45% B over 8 min; mobile phase A: 0.04% aqueous HCl, mobile phase B: acetonitrile). 5-(2-Aminopropoxy)-2-methyl-N-(1-(naphthalen-1-yl)cyclopropyl)benzamide (50.9 mg, 124 μmol, 73% yield, HCl salt) was obtained as a white solid. M+H⁺=375.1 (LCMS); ¹H NMR (400 MHZ, DMSO-d₆) δ 9.18-9.06 (m, 1H), 8.69-8.61 (m, 1H), 7.94 (br d, J=7.9 Hz, 4H), 7.83 (t, J=7.4 Hz, 2H), 7.60-7.44 (m, 3H), 7.11-7.05 (m, 1H), 6.92-6.86 (m, 1H), 6.69-6.63 (m, 1H), 4.08-3.97 (m, 1H), 3.89-3.80 (m, 1H), 3.60-3.49 (m, 1H), 1.97 (s, 3H), 1.35 (br s, 2H), 1.27-1.16 (m, 5H).

### Example 72: 5-(2-Amino-3,3-dimethylbutoxy)-2-methyl-N-(1-(naphthalen-1-yl)cyclopropyl)benzamide (Compound 343)

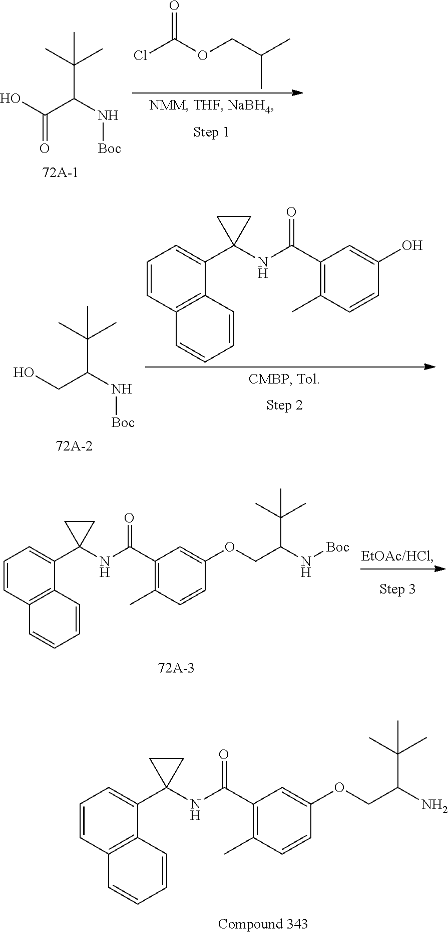

### Step 1: tert-Butyl(1-hydroxy-3,3-dimethylbutan-2-yl)carbamate (72A-2)

To a mixture of 2-(tert-butoxycarbonylamino)-3,3-dimethyl-butanoic acid (500 mg, 2.16 mmol, 1.0 eq) and NMM (219 mg, 2.16 mmol, 238 μL, 1.0 eq) in THF (4.0 mL) was added isobutyl carbonochloridate (295 mg, 2.16 mmol, 284 μL, 1.0 eq) at −10° C., the reaction mixture was stirred at −10° C. for 30 min. TLC indicated that the starting material was completely consumed. Then the reaction mixture was filtered. To the filtrate was added NaBH₄ (123 mg, 3.24 mmol, 1.5 eq) in portions at −10° C. The resulting reaction mixture was stirred at the same temperature for 30 min. TLC indicated that the starting material was completely consumed. The reaction mixture was poured into H₂O (30 mL) and extracted with EtOAc (30 mL×2). The combined organic layers were dried over Na₂SO₄, filtered, and concentrated under vacuum to give a residue which was purified by preparative TLC (EtOAc/petroleum ether=1/3, R_{f}=0.2). tert-Butyl(1-hydroxy-3,3-dimethylbutan-2-yl)carbamate (220 mg, 1.01 mmol, 47% yield) was obtained as a white solid.

### Step 2: tert-Butyl(3,3-dimethyl-1-(4-methyl-3-((1-(naphthalen-1-yl)cyclopropyl) carbamoyl)phenoxy) butan-2-yl)carbamate (72A-3)

A mixture of 5-hydroxy-2-methyl-N-[1-(1-naphthyl)cyclopropyl]benzamide (150 mg, 472 μmol, 1.0 eq), tert-butyl N-[1-(hydroxymethyl)-2,2-dimethyl-propyl]carbamate (205 mg, 945 μmol, 2.0 eq) and CMBP (171 mg, 709 μmol, 1.5 eq) in toluene (10 mL) was degassed and purged with N₂ three times. The mixture was stirred at 110° C. for 16 h. LCMS indicated that the starting material was completely consumed, and the desired mass was detected. The reaction mixture was allowed to cool to room temperature and concentrated under vacuum to give a residue which was purified by preparative TLC (EtOAc/petroleum ether=1/3, R_{f}=0.3). tert-Butyl(3,3-dimethyl-1-(4-methyl-3-((1-(naphthalene-1-yl)cyclopropyl)carbamoyl). phenoxy)butan-2-yl)carbamate (45 mg, 87.1 μmol, 18% yield) was obtained as a white solid. M+H⁺=517.3 (LCMS)

### Step 3: 5-(2-Amino-3,3-dimethylbutoxy)-2-methyl-N-(1-(naphthalen-1-yl)cyclopropyl)benzamide (Compound 343)

To a mixture of tert-butyl N-[2,2-dimethyl-1-[4-methyl-3-[1-(1-naphthyl)cyclopropyl]carbamoyl]phenoxy]methyl]propyl]carbamate (45.0 mg, 87.1 μmol, 1.0 eq) in EtOAc (500 μL) was added HCl/EtOAc (4 M, 1.0 mL). The reaction mixture was stirred at 25° C. for 1 h. LCMS indicated that the starting material was completely consumed, and the desired mass was detected. The mixture was concentrated under vacuum at 30° C. to give a residue which was purified by preparative HPLC (Phenomenex Gemini C18 column (80×30 mm, 3 μm); flow rate: 25 mL/min; gradient: 35%-60% B over 8 min; mobile phase A: 0.04% aqueous HCl, mobile phase B: acetonitrile). 5-(2-Amino-3,3-dimethyl-butoxy)-2-methyl-N-[1-(1-naphthyl)cyclopropyl]benzamide (18.0 mg, 45.0 μmol, 47% yield, HCl salt) was obtained as a white solid. M+H⁺=417.3 (LCMS); ¹H NMR (400 MHZ, DMSO-d₆) δ 9.11 (s, 1H), 8.66 (d, J=8.0 Hz, 1H), 8.02-7.76 (m, 6H), 7.64-7.40 (m, 3H), 7.08 (d, J=8.4 Hz, 1H), 6.93 (dd, J=2.8, 8.4 Hz, 1H), 6.71 (d, J=2.8 Hz, 1H), 4.16 (dd, J=3.2, 10.4 Hz, 1H), 3.91 (br t, J=9.6 Hz, 1H), 3.26-3.09 (m, 1H), 1.97 (s, 3H), 1.36 (br s, 2H), 1.18 (br s, 2H), 1.00 (s, 9H).

### Example 73: 5-(2-Amino-3-methoxypropoxy)-2-methyl-N-(1-(naphthalen-1-yl)cyclopropyl)benzamide (Compound 400)

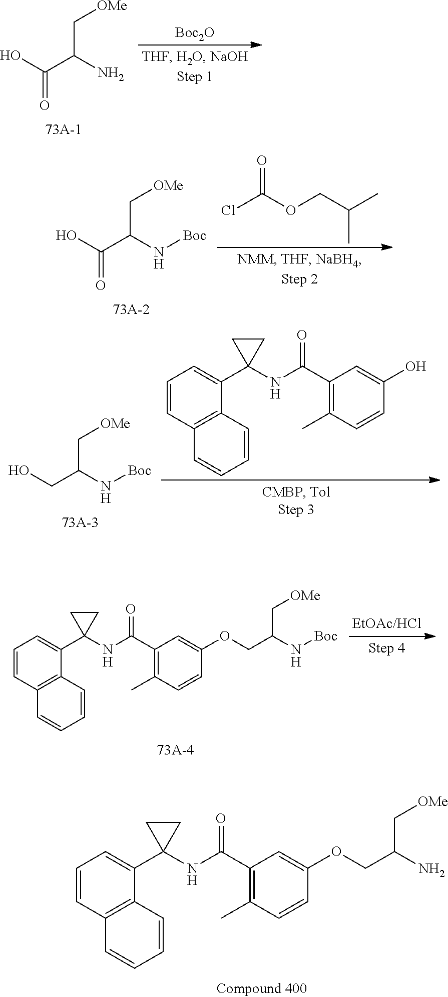

### Step 1: 2-((tert-Butoxycarbonyl)amino)-3-methoxypropanoic acid (73A-2)

To a mixture of 2-amino-3-methoxy-propanoic acid (500 mg, 4.20 mmol, 1.0 eq) and NaOH (336 mg, 8.39 mmol, 2.0 eq) in a mixture of THF (4.0 mL) and H₂O (2.0 mL) was added tert-butoxycarbonyl tert-butyl carbonate (1.01 g, 4.62 mmol, 1.1 eq) at 0° C. The reaction mixture was stirred at 25° C. for 16 h. LCMS indicated that the starting material was completely consumed, and the desired mass was detected. The mixture was concentrated under vacuum to give a residue which was purified by preparative TLC (EtOAc/petroleum ether=1/1, R_{f}=0.3). 2-((tert-Butoxycarbonyl)amino)-3-methoxy-propanoic acid (320 mg, 1.46 mmol, 35% yield) was obtained as white oil. ¹H NMR (400 MHZ, CDCl₃) δ 9.38-9.92 (m, 1H), 5.76 (br s, 1H), 4.19 (br s, 1H), 3.62-3.76 (m, 1H), 3.50 (br s, 1H), 3.26 (s, 3H), 1.35 (s, 9H).

### Step 2: tert-Butyl(1-hydroxy-3-methoxypropan-2-yl)carbamate (73A-3)

To a mixture of 2-(tert-butoxycarbonylamino)-3-methoxy-propanoic acid (220 mg, 1.00 mmol, 1.0 eq) and NMM (101 mg, 1.00 mmol, 1.0 eq) in THF (2.0 mL) was added isobutyl carbonochloridate (137 mg, 1.00 mmol, 1.0 eq) at −10° C. The reaction mixture was stirred at −10° C. for 30 min. TLC indicated that the starting material was completely consumed. The reaction mixture was filtered. To the filtrate was added NaBH₄ (57.0 mg, 1.51 mmol, 1.5 eq) at −10° C., the reaction mixture was stirred at −10° C. for 30 min. TLC indicated that the intermediate was completely consumed. The reaction mixture was poured into H₂O (30 mL) and extracted with EtOAc (30 mL×2). The combined organic layers were dried over Na₂SO₄, filtered, and concentrated under vacuum to give a residue which was purified by preparative TLC (EtOAc/petroleum ether=1/3, R_{f}=0.1). tert-Butyl(1-hydroxy-3-methoxypropan-2-yl)carbamate (100 mg, 487 μmol, 49% yield) was obtained as a colorless oil.

### Step 3: tert-Butyl(1-methoxy-3-(4-methyl-3-((1-(naphthalen-1-yl)cyclopropyl)carbamoyl)phenoxy)propan-2-yl)carbamate (73A-4)

A mixture of 5-hydroxy-2-methyl-N-[1-(1-naphthyl)cyclopropyl]benzamide (150 mg, 473 μmol, 1.0 eq), tert-butyl N-[1-(hydroxymethyl)-2-methoxy-ethyl]carbamate (97.0 mg, 473 μmol, 1.0 eq) and CMBP (114 mg, 473 μmol, 1.0 eq) in toluene (4.0 mL) was degassed and purged with N₂ three times. The mixture was stirred at 110° C. for 16 h. LCMS indicated that the starting material was completely consumed, and the desired mass was detected. The reaction mixture was allowed to cool to room temperature, concentrated under vacuum to give a residue which was purified by preparative TLC (EtOAc/petroleum ether=1/1, R_{f}=0.7). tert-Butyl(1-methoxy-3-(4-methyl-3-((1-(naphthalen-1-yl)cyclopropyl)carbamoyl)phenoxy) propan-2-yl)carbamate (140 mg, 277 μmol, 59% yield) was obtained as a white solid. M+H⁺=505.2 (LCMS).

### Step 4: 5-(2-Amino-3-methoxypropoxy)-2-methyl-N-(1-(naphthalen-1-yl)cyclopropyl)benzamide (Compound 400)

To a solution of tert-butyl(1-methoxy-3-(4-methyl-3-((1-(naphthalen-1-yl)cyclopropyl) carbamoyl)phenoxy) propan-2-yl)carbamate (140 mg, 277 μmol, 1.0 eq) in EtOAc (1.0 mL) was added HCl/EtOAc (4 M, 1.0 mL). The mixture was stirred at 25° C. for 1 h. LCMS indicated that the starting material was completely consumed, and the desired mass was detected. The reaction mixture was concentrated under vacuum to give a residue which was purified by preparative HPLC (Phenomenex Luna C18 column (80×30 mm, 3 μm); flow rate: 25 mL/min; gradient: 30%-55% B over 8 min; mobile phase A: 0.04% aqueous HCl, mobile phase B: acetonitrile). 5-(2-amino-3-methoxy-propoxy)-2-methyl-N-[1-(1-naphthyl)cyclopropyl]benzamide (92.7 mg, 210 μmol, 74% yield, HCl salt) was obtained as a white solid. M+H⁺=405.1 (LCMS); ¹H NMR (400 MHZ, CD₃OD) δ 9.19 (s, 1H), 8.58 (d, J=8.4 Hz, 1H), 7.94-7.76 (m, 3H), 7.60-7.38 (m, 3H), 7.08 (d, J=8.4 Hz, 1H), 6.90 (dd, J=2.8, 8.4 Hz, 1H), 6.69 (d, J=2.8 Hz, 1H), 4.15-3.99 (m, 2H), 3.74-3.57 (m, 3H), 3.41 (s, 3H), 2.00 (s, 3H), 1.51-1.40 (m, 2H), 1.34-1.28 (m, 2H).

### Example 74: 5-(2-Amino-2-phenylethoxy)-2-methyl-N-(1-(naphthalen-1-yl)cyclopropyl)benzamide (Compound 367)

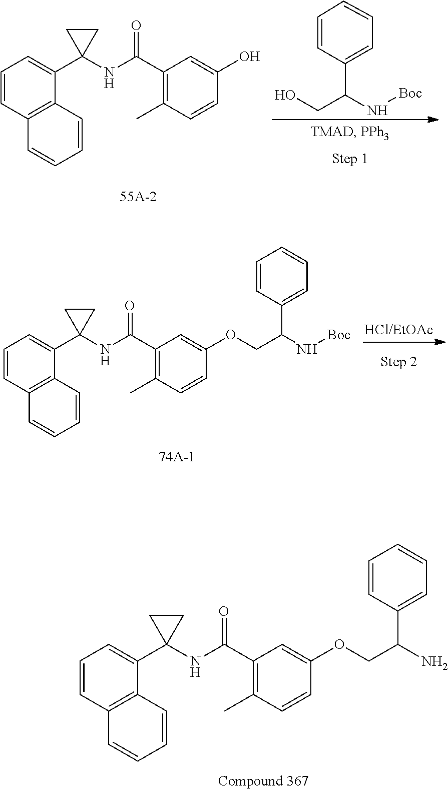 [SMILES: Cc1ccc(O)cc1C(=O)NC1(c2cccc3ccccc23)CC1 ; CC(C)(C)OC(=O)NC(CO)c1ccccc1 ; Cc1ccc(OCC(NC(=O)OC(C)(C)C)c2ccccc2)cc1C(=O)NC1(c2cccc3ccccc23)CC1 ; Cc1ccc(OCC(N)c2ccccc2)cc1C(=O)NC1(c2cccc3ccccc23)CC1]

### Step 1: tert-Butyl(2-(4-methyl-3-((1-(naphthalen-1-yl)cyclopropyl)carbamoyl)phenoxy)-1-phenylethyl)carbamate (74A-1)

A mixture of 5-hydroxy-2-methyl-N-(1-(naphthalen-1-yl)cyclopropyl)benzamide (200 mg, 630 μmol, 1.0 eq) and tert-butyl(2-hydroxy-1-phenylethyl)carbamate (141 mg, 630 μmol, 1.0 eq) in toluene (10 mL) was degassed and purged with N₂ three times. To the mixture were added TMAD (326 mg, 1.89 mmol, 3.0 eq), PPh₃ (496 mg, 1.89 mmol, 3.0 eq), and the mixture was stirred at 100° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (4.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. tert-Butyl(2-(4-methyl-3-((1-(naphthalen-1-yl)cyclopropyl)carbamoyl)phenoxy)-1-phenylethyl)carbamate (150 mg, 280 μmol, 44% yield) was obtained as a yellow oil. M+H⁺=537.4 (LCMS).

### Step 2: 5-(2-Amino-2-phenylethoxy)-2-methyl-N-(1-(naphthalen-1-yl)cyclopropyl)benzamide (Compound 367)

To a solution of tert-butyl(2-(4-methyl-3-((1-(naphthalen-1-yl)cyclopropyl)carbamoyl) phenoxy)-1-phenylethyl)carbamate (150 mg, 280 μmol, 1.0 eq) in EtOAc (2.0 mL) was added HCl/EtOAc (4 M, 2.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 10%-50% B over 8 min; mobile phase A: 0.04% aqueous HCl, mobile phase B: acetonitrile). 5-(2-Amino-2-phenylethoxy)-2-methyl-N-(1-(naphthalen-1-yl)cyclopropyl)benzamide (73.2 mg, 155 μmol, 55% yield, HCl salt) was obtained as a white solid. M+H⁺=437.1 (LCMS); ¹H NMR (400 MHZ, DMSO-d₆) δ 9.11 (s, 1H), 8.63 (br s, 4H), 7.98-7.90 (m, 1H), 7.82 (t, J=8.3 Hz, 2H), 7.59-7.41 (m, 8H), 7.06 (d, J=8.6 Hz, 1H), 6.90 (dd, J=2.4, 8.3 Hz, 1H), 6.69 (d, J=2.4 Hz, 1H), 4.74-4.65 (m, 1H), 4.26-4.12 (m, 2H), 1.96 (s, 3H), 1.34 (br s, 2H), 1.17 (br s, 2H).

### Example 75: 5-(2-Amino-3-phenylpropoxy)-2-methyl-N-(1-(naphthalen-1-yl)cyclopropyl)benzamide (Compound 322)

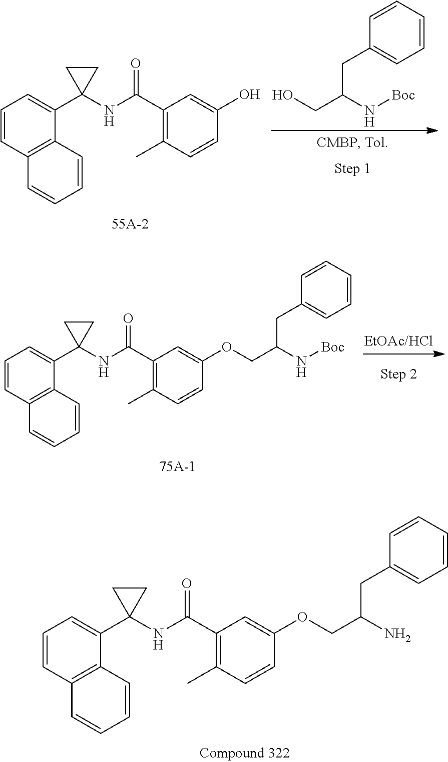

### Step 1: tert-Butyl(1-(4-methyl-3-((1-(naphthalen-1-yl)cyclopropyl)carbamoyl)phenoxy)-3-phenylpropan-2-yl)carbamate (75A-1)

A mixture of 5-hydroxy-2-methyl-N-[1-(1-naphthyl)cyclopropyl]benzamide (80.0 mg, 252 μmol, 1.0 eq), tert-butyl N-(1-benzyl-2-hydroxy-ethyl)carbamate (63.3 mg, 252 μmol, 1.0 eq) and CMBP (91.2 mg, 378 μmol, 1.5 eq) in toluene (4.0 mL) was degassed and purged with N₂ atmosphere, and then the mixture was stirred at 110° C. for 16 h. LCMS indicated that the starting material was completely consumed, and the desired mass was detected. The mixture was allowed to cool to room temperature and concentrated under vacuum to give a residue which was purified by preparative TLC (EtOAc/petroleum ether=1/3, R_{f}=0.4). tert-Butyl(1-(4-methyl-3-((1-(naphthalen-1-yl)cyclopropyl)carbamoyl)phenoxy)-3-phenylpropan-2-yl)carbamate (80.0 mg, 145 μmol, 58% yield) was obtained as a white solid. M+H⁺=551.2 (LCMS).

### Step 2: 5-(2-Amino-3-phenylpropoxy)-2-methyl-N-(1-(naphthalen-1-yl)cyclopropyl)benzamide (Compound 322)

To a mixture of tert-butyl N-[1-benzyl-2-[4-methyl-3-[1-(1-naphthyl)cyclopropyl]carbamoyl]phenoxy]ethyl]carbamate (70.0 mg, 127 μmol, 1.0 eq) in EtOAc (1.0 mL) was added HCl/EtOAc (4 M, 2.0 mL). The reaction mixture was stirred at 25° C. for 1 h. LCMS indicated that the starting material was completely consumed, and the desired mass was detected. The mixture was concentrated under vacuum to give a residue which was purified by preparative HPLC (Phenomenex Luna C18 column (80×30 mm, 3 μm); flow rate: 25 mL/min; gradient: 35%-60% B over 8 min; mobile phase A: 0.04% aqueous HCl, mobile phase B: acetonitrile). 5-(2-Amino-3-phenyl-propoxy)-2-methyl-N-[1-(1-naphthyl)cyclopropyl]benzamide (25.0 mg, 51.7 μmol, 41% yield, HCl salt) was obtained as a white solid. M+H⁺=451.1 (LCMS); ¹H NMR (400 MHZ, DMSO-d₆) δ 9.11 (s, 1H), 8.64 (br d, J=8.4 Hz, 1H), 8.25 (br s, 3H), 7.94 (br d, J=7.8 Hz, 1H), 7.83 (br t, J=7.8 Hz, 2H), 7.60-7.40 (m, 3H), 7.36-7.17 (m, 5H), 7.05 (br d, J=8.4 Hz, 1H), 6.88-6.78 (m, 1H), 6.63 (br s, 1H), 4.00-3.61 (m, 3H), 3.14-2.88 (m, 2H), 1.95 (s, 3H), 1.43-1.04 (m, 4H).

### Example 76: 5-(2-Amino-2-methylpropoxy)-2-methyl-N-(1-(naphthalen-1-yl)cyclopropyl)benzamide (Compound 352)

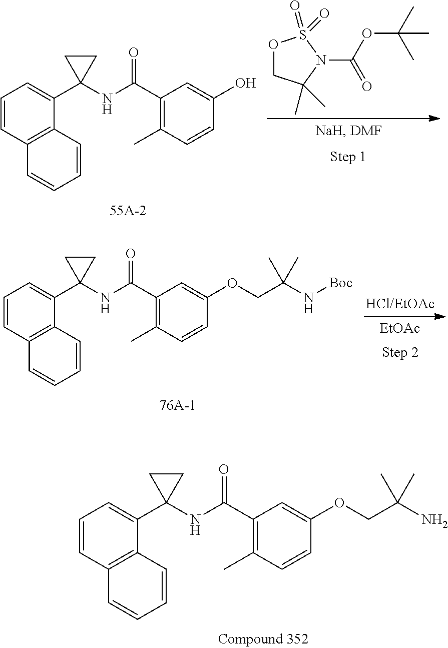

### Step 1: tert-Butyl(2-methyl-1-(4-methyl-3-((1-(naphthalen-1-yl)cyclopropyl)carbamoyl) phenoxy)propan-2-yl)carbamate (76A-1)

To a solution of 5-hydroxy-2-methyl-N-(1-(naphthalen-1-yl)cyclopropyl)benzamide (100 mg, 315 μmol, 1.0 eq) in DMF (5.0 mL) was added sodium hydride (18.9 mg, 473 μmol, 60% purity, 1.5 eq) under a N₂ atmosphere, the mixture was stirred at 0° C. for 30 min, then tert-butyl 4,4-dimethyl-1,2,3-oxathiazolidine-3-carboxylate 2,2-dioxide (119 mg, 473 μmol, 1.5 eq) was added. The resulting mixture was stirred at 70° C. for 2 h. TLC indicated that the starting material was completely consumed. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by preparative TLC (EtOAc/petroleum ether=1/1, R_{f}=0.7). tert-Butyl(2-methyl-1-(4-methyl-3-((1-(naphthalen-1-yl)cyclopropyl)carbamoyl)phenoxy) propan-2-yl)carbamate (140 mg, 287 μmol, 91% yield) was obtained as a yellow solid. 1H NMR (400 MHZ, CDCl₃) δ 8.48 (d, J=8.6 Hz, 1H), 7.95 (d, J=7.0 Hz, 1H), 7.90 (d, J=8.1 Hz, 1H), 7.81 (d, J=8.3 Hz, 1H), 7.63-7.56 (m, 1H), 7.54-7.45 (m, 2H), 6.99 (d, J=8.5 Hz, 1H), 6.79 (dd, J=2.6, 8.3 Hz, 1H), 6.71 (d, J=2.5 Hz, 1H), 6.48 (br s, 1H), 3.84 (s, 2H), 2.10 (s, 3H), 1.46-1.38 (m, 13H), 1.35 (s, 6H).

### Step 2: 5-(2-Amino-2-methylpropoxy)-2-methyl-N-(1-(naphthalen-1-yl)cyclopropyl)benzamide (Compound 352)

To a solution of tert-butyl(2-methyl-1-(4-methyl-3-((1-(naphthalen-1-yl)cyclopropyl) carbamoyl)phenoxy) propan-2-yl)carbamate (80.0 mg, 164 μmol, 1.0 eq) in EtOAc (1.0 mL) was added HCl/EtOAc (4 M, 3.0 mL). The resulting mixture was stirred at 20° C. for 1 h. LCMS indicated that the starting material was completely consumed, and the desired mass was detected. The reaction mixture was concentrated under vacuum to give a residue which was purified by preparative HPLC (Phenomenex Luna C18 column (80×30 mm, 3 μm); flow rate: 25 mL/min; gradient: 5%-50% B over 8 min; mobile phase A: 0.04% aqueous HCl, mobile phase B: acetonitrile) to give 5-(2-amino-2-methylpropoxy)-2-methyl-N-(1-(naphthalen-1-yl)cyclopropyl)benzamide (38.6 mg, 90.7 μmol, 55% yield) as a brown solid. M+H⁺=389.1 (LCMS); ¹H NMR (400 MHZ, DMSO-d₆) δ 9.13 (s, 1H), 8.66 (d, J=8.4 Hz, 1H), 8.18 (br d, J=1.9 Hz, 3H), 7.93 (d, J=8.0 Hz, 1H), 7.86-7.77 (m, 2H), 7.64-7.39 (m, 3H), 7.07 (d, J=8.5 Hz, 1H), 6.89 (dd, J=2.6, 8.3 Hz, 1H), 6.69 (d, J=2.6 Hz, 1H), 3.87 (s, 2H), 1.96 (s, 3H), 1.36 (s, 2H), 1.29 (s, 6H), 1.22-1.12 (m, 2H).

### Example 77: 5-(2-Aminoethoxy)-2-methyl-N-(1-(naphthalen-1-yl)cyclopropyl)benzamide (Compound 256)

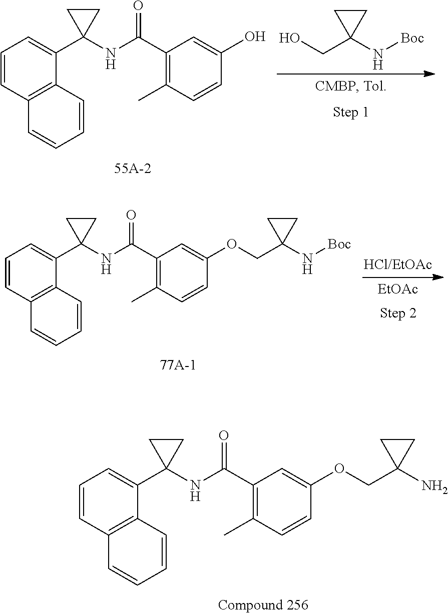 [SMILES: "EtOAc" ; Cc1ccc(O)cc1C(=O)NC1(c2cccc3ccccc23)CC1 ; CC(C)(C)OC(=O)NC1(CO)CC1 ; Cc1ccc(OCC2(NC(=O)OC(C)(C)C)CC2)cc1C(=O)NC1(c2cccc3ccccc23)CC1 ; Cc1ccc(OCC2(N)CC2)cc1C(=O)NC1(c2cccc3ccccc23)CC1]

### Step 1: tert-Butyl(1-((4-methyl-3-((1-(naphthalen-1-yl)cyclopropyl)carbamoyl) phenoxy)methyl)cyclopropyl)carbamate (77A-1)

A mixture of 5-hydroxy-2-methyl-N-(1-(naphthalen-1-yl)cyclopropyl)benzamide (200 mg, 586 μmol, 1.0 eq) and tert-butyl(1-(hydroxymethyl)cyclopropyl)carbamate (165 mg, 879 μmol, 1.5 eq) in toluene (2.0 mL) was degassed and purged with N₂ three times. To the mixture was added CMBP (212 mg, 879 μmol, 1.5 eq) in portions at 25° C. The resulting mixture was stirred at 110° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by preparative TLC (EtOAc/petroleum ether=1/1, R_{f}=0.5). tert-Butyl(1-((4-methyl-3-((1-(naphthalen-1-yl)cyclopropyl)carbamoyl) phenoxy)methyl)cyclopropyl)carbamate (150 mg, 308 μmol, 53% yield) was obtained as a yellow oil. M+H⁺=487.2 (LCMS).

### Step 2: 5-(2-Aminoethoxy)-2-methyl-N-(1-(naphthalen-1-yl)cyclopropyl)benzamide (Compound 256)

To a stirred solution of tert-butyl(1-((4-methyl-3-((1-(naphthalen-1-yl)cyclopropyl)carbamoyl)phenoxy)methyl)cyclopropyl)carbamate (150 mg, 308 μmol, 1.0 eq) in EtOAc (1.0 mL) was added HCl/EtOAc (4 M, 5.0 mL). The mixture was stirred at 25° C. for 1 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 15%-60% B over 8 min; mobile phase A: 0.04% aqueous HCl, mobile phase B: acetonitrile). 5-(2-Aminoethoxy)-2-methyl-N-(1-(naphthalen-1-yl)cyclopropyl)benzamide (69.2 mg, 179 μmol, 68% yield, HCl salt) was obtained as a white solid. M+H⁺=387.1 (LCMS); ¹H NMR (400 MHZ, CDCl₃) δ 8.71 (br s, 3H), 8.59 (br d, J=8.3 Hz, 1H), 7.95 (d, J=7.1 Hz, 1H), 7.85 (d, J=8.1 Hz, 1H), 7.76 (d, J=8.2 Hz, 1H), 7.51-7.41 (m, 3H), 7.37 (br s, 1H), 6.82-6.72 (m, 2H), 6.68 (br d, J=7.2 Hz, 1H), 3.80 (s, 2H), 1.99 (s, 3H), 1.53 (br s, 2H), 1.34 (br s, 2H), 1.19 (s, 2H), 0.68 (br s, 2H).

### Example 78: 5-(Azetidin-2-ylmethoxy)-2-methyl-N-(1-(naphthalen-1-yl)cyclopropyl)benzamide (Compound 274)

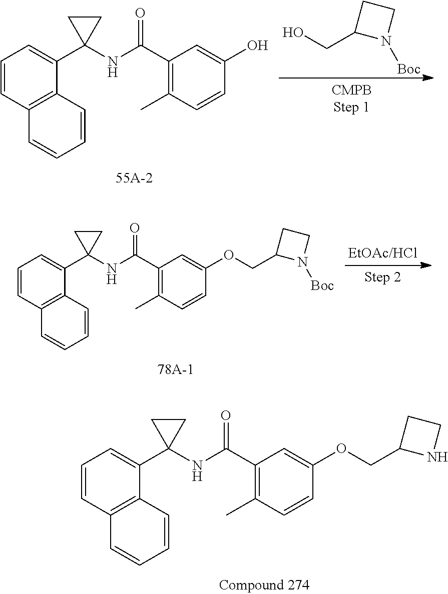 [SMILES: Cc1ccc(O)cc1C(=O)NC1(c2cccc3ccccc23)CC1 ; CC(C)(C)OC(=O)N1CCC1CO ; Cc1ccc(OCC2CCN2C(=O)OC(C)(C)C)cc1C(=O)NC1(c2cccc3ccccc23)CC1 ; Cc1ccc(OCC2CCN2)cc1C(=O)NC1(c2cccc3ccccc23)CC1]

### Step 1: tert-Butyl 2-((4-methyl-3-((1-(naphthalen-1-yl)cyclopropyl)carbamoyl)phenoxy)methyl)azetidine-1-carboxylate (78A-1)

A mixture of 5-hydroxy-2-methyl-N-(1-(naphthalen-1-yl)cyclopropyl)benzamide (300 mg, 945 μmol, 1.0 eq) and tert-butyl 2-(hydroxymethyl)azetidine-1-carboxylate (177 mg, 945 μmol, 1.0 eq) in toluene (15 mL) was added CMBP (342 mg, 1.42 mmol, 1.5 eq). The mixture was degassed and purged with N₂ three times and then the mixture was stirred at 100° C. for 16 h under a N₂ atmosphere. LCMS indicated that the starting material was completely consumed, and the desired mass was detected. The reaction mixture was allowed to cool to room temperature, poured into H₂O (15 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. tert-Butyl 2-((4-methyl-3-((1-(naphthalen-1-yl)cyclopropyl)carbamoyl)phenoxy)methyl)azetidine-1-carboxylate (300 mg, 617 μmol, 65% yield) was obtained as a colorless oil. M+H⁺=387.1 (LCMS); ¹H NMR (400 MHZ, CDCl₃) δ 8.50 (d, J=8.4 Hz, 1H), 7.96 (d, J=7.1 Hz, 1H), 7.90 (d, J=8.2 Hz, 1H), 7.80 (d, J=8.1 Hz, 1H), 7.61-7.55 (m, 1H), 7.54-7.45 (m, 2H), 7.00 (d, J=8.4 Hz, 1H), 6.82 (dd, J=2.6, 8.3 Hz, 1H), 6.75 (d, J=2.6 Hz, 1H), 6.64 (s, 1H), 4.43 (br dd, J=2.9, 5.1 Hz, 1H), 4.22-4.15 (m, 1H), 4.04-3.96 (m, 1H), 3.87 (t, J=7.6 Hz, 2H), 2.12 (s, 3H), 2.06 (s, 2H), 1.57 (s, 2H), 1.40 (br s, 2H), 1.36 (s, 9H).

### Step 2: 5-(Azetidin-2-ylmethoxy)-2-methyl-N-(1-(naphthalen-1-yl)cyclopropyl)benzamide (Compound 274)

To a solution of tert-butyl 2-((4-methyl-3-((1-(naphthalen-1-yl)cyclopropyl)carbamoyl) phenoxy)methyl)azetidine-1-carboxylate (100 mg, 206 μmol, 1.0 eq) in EtOAc (2.0 mL) was added HCl/EtOAc (4 M, 2.00 mL). The mixture was stirred at 20° C. for 2 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 20%-50% B over 8 min; mobile phase A: 0.04% aqueous HCl, mobile phase B: acetonitrile). 5-(Azetidin-2-ylmethoxy)-2-methyl-N-(1-(naphthalen-1-yl)cyclopropyl)benzamide (30.1 mg, 71.2 μmol, 35% yield, HCl salt) was obtained as a white solid. M+H⁺=387.2 (LCMS); ¹H NMR (400 MHZ, DMSO-d₆) δ 9.37 (s, 1H), 9.15 (m, 2H), 8.67 (d, J=8.3 Hz, 1H), 7.93 (d, J=7.8 Hz, 1H), 7.86-7.78 (m, 2H), 7.62-7.42 (m, 3H), 7.08 (d, J=8.6 Hz, 1H), 6.90 (dd, J=2.7, 8.4 Hz, 1H), 6.70 (d, J=2.7 Hz, 1H), 4.63 (br d, J=6.0 Hz, 1H), 4.29 (dd, J=7.5, 11.2 Hz, 1H), 4.13 (dd, J=3.2, 11.1 Hz, 1H), 3.95-3.76 (m, 2H), 2.47-2.26 (m, 2H), 1.97 (s, 3H), 1.36 (s, 2H), 1.23-1.13 (m, 2H).

### Example 79: (S)-2-Methyl-5-((1-methylazetidin-2-yl)methoxy)-N-(1-(naphthalen-1-yl)cyclopropyl)benzamide (Compound 310)

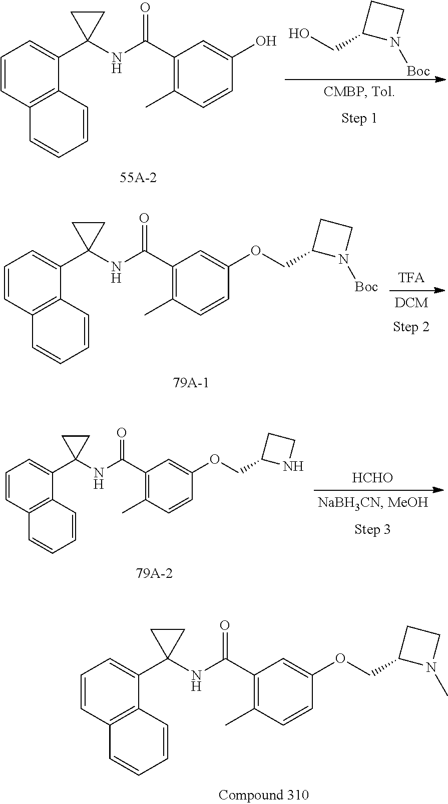

### Step 1: (S)-tert-Butyl 2-((4-methyl-3-((1-(naphthalen-1-yl)cyclopropyl)carbamoyl)phenoxy)methyl)azetidine-1-carboxylate (79A-1)

To a mixture of 5-hydroxy-2-methyl-N-(1-(naphthalen-1-yl)cyclopropyl)benzamide (100 mg, 315 μmol, 1.0 eq) and(S)-tert-butyl 2-(hydroxymethyl)azetidine-1-carboxylate (59.0 mg, 315 μmol, 1.0 eq) in toluene (5.0 mL) was added CMBP (114 mg, 473 μmol, 1.5 eq). The resulting mixture was degassed and purged with N₂ three times and then was stirred at 110° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by preparative TLC (EtOAc/petroleum ether=1/1, R_{f}=0.6). (S)-tert-Butyl 2-((4-methyl-3-((1-(naphthalen-1-yl)cyclopropyl)carbamoyl)phenoxy)methyl) azetidine-1-carboxylate (130 mg, 267 μmol, 85% yield) was obtained as a yellow solid. M+H⁺=487.2 (LCMS); ¹H NMR (400 MHZ, CDCl₃) δ 8.50 (d, J=8.2 Hz, 1H), 7.96 (d, J=7.1 Hz, 1H), 7.90 (d, J=8.1 Hz, 1H), 7.81 (d, J=8.3 Hz, 1H), 7.64-7.43 (m, 3H), 7.05-6.95 (m, 1H), 6.88-6.73 (m, 2H), 6.64 (br s, 1H), 4.48-4.38 (m, 1H), 4.22-4.15 (m, 1H), 4.03-3.96 (m, 1H), 3.87 (br t, J=7.2 Hz, 2H), 2.38-2.17 (m, 2H), 2.12 (s, 3H), 1.57-1.53 (m, 2H), 1.46-1.31 (m, 11H).

### Step 2: (S)-5-(Azetidin-2-ylmethoxy)-2-methyl-N-(1-(naphthalen-1-yl)cyclopropyl)benzamide (79A-2)

To a solution of(S)-tert-butyl 2-((4-methyl-3-((1-(naphthalen-1-yl)cyclopropyl)carbamoyl) phenoxy)methyl)azetidine-1-carboxylate (130 mg, 267 μmol, 1.0 eq) in DCM (10 mL) was added TFA (2.5 mL). The resulting mixture was stirred at 25° C. for 1 h. LCMS indicated that the starting material was completely consumed, and the desired mass was detected. The reaction mixture was concentrated under vacuum to give crude product(S)-5-(azetidin-2-ylmethoxy)-2-methyl-N-(1-(naphthalen-1-yl)cyclopropyl)benzamide (100 mg, 199 μmol, 78% yield, TFA salt) as a yellow gum, which was used in the next step without any further purification. M+H⁺=387.2 (LCMS).

### Step 3: (S)-2-Methyl-5-((1-methylazetidin-2-yl)methoxy)-N-(1-(naphthalen-1-yl)cyclopropyl)benzamide (Compound 310)

To a stirred solution of(S)-5-(azetidin-2-ylmethoxy)-2-methyl-N-(1-(naphthalen-1-yl)cyclopropyl)benzamide (100 mg, 259 μmol, 1.0 eq, TFA salt) in MeOH (5.0 mL) were added TEA (26.2 mg, 259 μmol, 36.0 μL, 1.0 eq) and formaldehyde (42.0 mg, 517 μmol, 38.5 μL, 37% purity in aqueous, 2.0 eq). The resulting mixture was treated with a small amount of AcOH to adjust the pH to 6, and then NaBH₃CN (32.5 mg, 517 μmol, 2.0 eq) was added. The reaction mixture was stirred at 20° C. for 16 h. LCMS indicated that the starting material was completely consumed, and the desired mass was detected. The reaction mixture was poured into H₂O (5.0 mL) and extracted with EtOAc (5.0 mL×5). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 25%-60% B over 8 min; mobile phase A: 10 mM aqueous NH₄HCO₃, mobile phase B: acetonitrile) to give(S)-2-methyl-5-((1-methylazetidin-2-yl)methoxy)-N-(1-(naphthalen-1-yl)cyclopropyl)benzamide (44.3 mg, 110 μmol, 43% yield) as a white solid. M+H⁺=401.1 (LCMS); ¹H NMR (400 MHZ, DMSO-d₆) δ 9.07 (s, 1H), 8.66 (br d, J=8.4 Hz, 1H), 7.93 (d, J=7.9 Hz, 1H), 7.82 (t, J=7.6 Hz, 2H), 7.61-7.41 (m, 3H), 7.02 (d, J=8.5 Hz, 1H), 6.82 (dd, J=2.5, 8.3 Hz, 1H), 6.60 (d, J=2.5 Hz, 1H), 3.85 (d, J=5.4 Hz, 2H), 3.27-3.15 (m, 2H), 2.79-2.67 (m, 1H), 2.21 (s, 3H), 2.04-1.90 (m, 4H), 1.89-1.77 (m, 1H), 1.36 (br s, 2H), 1.17 (br s, 2H).

### Example 80: (R)-2-Methyl-5-((1-methylazetidin-2-yl)methoxy)-N-(1-(naphthalen-1-yl)cyclopropyl)benzamide (Compound 309)

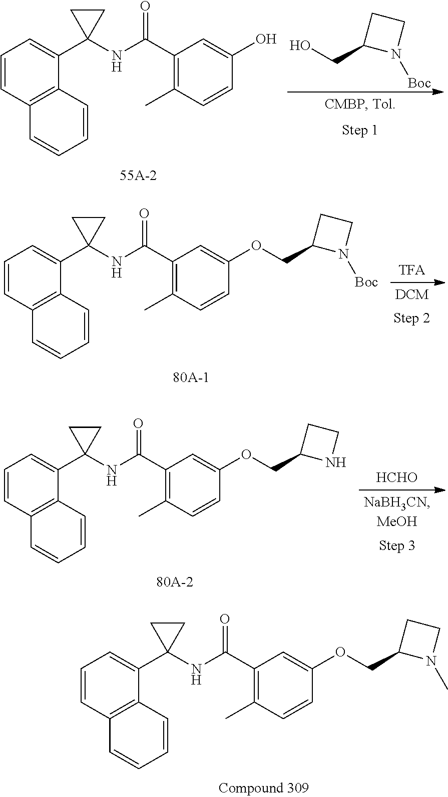

### Step 1: (R)-tert-Butyl 2-((4-methyl-3-((1-(naphthalen-1-yl)cyclopropyl)carbamoyl) phenoxy)methyl)azetidine-1-carboxylate (80A-1)

A mixture of 5-hydroxy-2-methyl-N-(1-(naphthalen-1-yl)cyclopropyl)benzamide (100 mg, 315 μmol, 1.0 eq) and (R)-tert-butyl 2-(hydroxymethyl)azetidine-1-carboxylate (59.0 mg, 315 μmol, 3.97 μL, 1.0 eq) in anhydrous toluene (5.0 mL) was degassed and purged with N₂ three times. To the mixture was added CMBP (114 mg, 473 μmol, 1.5 eq) at 20° C. The mixture was stirred at 110° C. for 16 h. LCMS indicated that the starting material was completely consumed, and the desired mass was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. (R)-tert-Butyl 2-((4-methyl-3-((1-(naphthalen-1-yl)cyclopropyl)carbamoyl)phenoxy)methyl)azetidine-1-carboxylate (280 mg, 575 μmol, 91% yield) was obtained as a white solid. M+H⁺=487.3 (LCMS); ¹H NMR (400 MHZ, DMSO-d₆) δ 9.08 (s, 1H), 8.66 (d, J=8.4 Hz, 1H), 7.93 (d, J=7.5 Hz, 1H), 7.86-7.79 (m, 2H), 7.60-7.42 (m, 3H), 7.03 (d, J=8.4 Hz, 1H), 6.86 (dd, J=2.6, 8.4 Hz, 1H), 6.65 (d, J=2.6 Hz, 1H), 4.43-4.33 (m, 1H), 4.13 (dd, J=4.9, 10.3 Hz, 1H), 3.98 (dd, J=2.9, 10.3 Hz, 1H), 3.72 (br d, J=6.6 Hz, 2H), 2.31-2.21 (m, 1H), 2.12-2.01 (m, 1H), 1.95 (s, 3H), 1.38-1.35 (m, 2H), 1.35-1.26 (m, 9H), 1.17 (s, 2H).

### Step 2: (R)-5-(Azetidin-2-ylmethoxy)-2-methyl-N-(1-(naphthalen-1-yl)cyclopropyl)benzamide (80A-2)

To a stirred solution of (R)-tert-butyl 2-((4-methyl-3-((1-(naphthalen-1-yl)cyclopropyl) carbamoyl)phenoxy)methyl)azetidine-1-carboxylate (280 mg, 575 μmol, 1.0 eq) in DCM (5.0 mL) was added TFA (5.6 mL). The mixture was stirred at 20° C. for 1 h. LCMS indicated that the starting material was completely consumed, and the desired mass was detected. The mixture was concentrated under vacuum at 30° C. to give the crude product (R)-5-(azetidin-2-ylmethoxy)-2-methyl-N-(1-(naphthalen-1-yl)cyclopropyl)benzamide (280 mg, crude, TFA salt) as a white solid. M+H⁺==387.1 (LCMS).

### Step 3: (R)-2-Methyl-5-((1-methylazetidin-2-yl)methoxy)-N-(1-(naphthalen-1-yl)cyclopropyl)benzamide (Compound 309)

To a solution of (R)-5-(azetidin-2-ylmethoxy)-2-methyl-N-(1-(naphthalen-1-yl)cyclopropyl)benzamide (140 mg, 280 μmol, 1.0 eq, TFA salt) in MeOH (6.0 mL) was added TEA (39.0 μL), followed by the addition of formaldehyde (45.4 mg, 559 μmol, 41.7 μL, 37% purity in water, 2.0 eq). The resulting mixture was adjusted to pH 6 with a small amount of AcOH. The mixture was stirred at 20° C. for 30 min, then NaBH₃CN (35.2 mg, 559 mmol, 2.0 eq) was added. The reaction mixture was stirred at 20° C. for another 16 h. LCMS indicated that the starting material was completely consumed, and the desired mass was detected. The reaction mixture was poured into H₂O (10 mL) and extracted with EtOAc (5.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 30%-60% B over 8 min; mobile phase A: 10 mM aqueous NH₄HCO₃, mobile phase B: acetonitrile). (R)-2-Methyl-5-((1-methylazetidin-2-yl)methoxy)-N-(1-(naphthalen-1-yl)cyclopropyl)benzamide (30.3 mg, 75.7 μmol, 27% yield) was obtained as a colorless oil. M+H⁺=401.1 (LCMS); ¹H NMR (400 MHZ, DMSO-d₆) δ 9.06 (s, 1H), 8.66 (d, J=8.3 Hz, 1H), 7.95-7.89 (m, 1H), 7.85-7.78 (m, 2H), 7.59-7.42 (m, 3H), 7.01 (d, J=8.5 Hz, 1H), 6.82 (dd, J=2.8, 8.4 Hz, 1H), 6.59 (d, J=2.8 Hz, 1H), 3.85 (d, J=5.5 Hz, 2H), 3.27-3.16 (m, 2H), 2.75-2.68 (m, 1H), 2.20 (s, 3H), 1.98-1.90 (m, 4H), 1.89-1.78 (m, 1H), 1.38-1.33 (m, 2H), 1.19-1.13 (m, 2H).

### Example 81: 2-Methyl-5-((1-methylazetidin-2-yl)methoxy)-N-(3-(naphthalen-1-yl)oxetan-3-yl)benzamide (Compound 387)

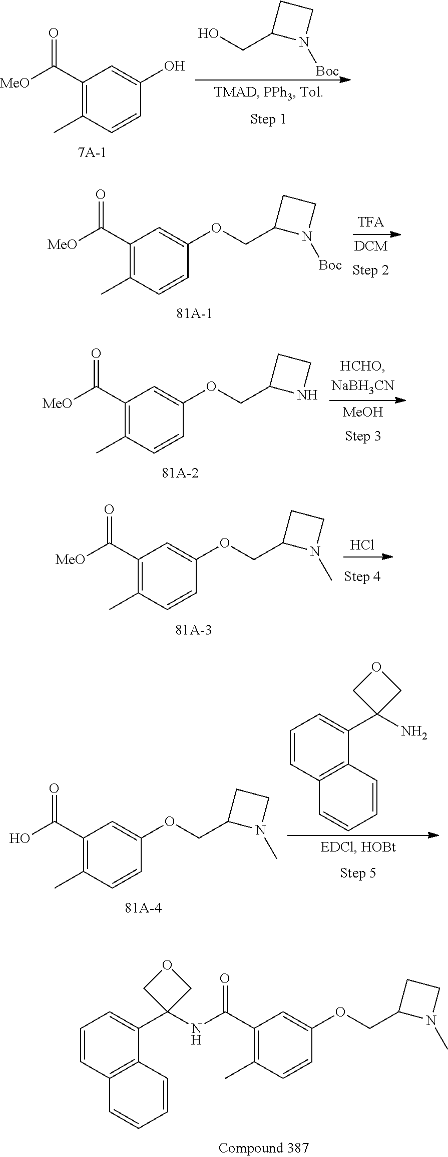

### Step 1: tert-Butyl 2-((3-(methoxycarbonyl)-4-methylphenoxy)methyl)azetidine-1-carboxylate (81A-1)

To a solution of methyl 5-hydroxy-2-methylbenzoate (500 mg, 3.01 mmol, 1.0 eq) and tert-butyl 2-(hydroxymethyl)azetidine-1-carboxylate (592 mg, 3.16 mmol, 1.1 eq) in toluene (15 mL) were added TMAD (1.55 g, 9.03 mmol, 3.0 eq) and PPh₃ (2.37 g, 9.03 mmol, 3.0 eq). The mixture was stirred at 100° C. for 16 h under a N₂ atmosphere. LCMS indicated that the starting material was completely consumed, and the desired mass was detected. The reaction mixture was poured into H₂O (30 mL) and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 0/1 to 1/4. tert-Butyl 2-((3-(methoxycarbonyl)-4-methylphenoxy)methyl)azetidine-1-carboxylate (900 mg, 2.68 mmol, 89% yield) was obtained as a yellow oil. M−56+H⁺=280.0 (LCMS); ¹H NMR (400 MHZ, CDCl₃) δ 7.50-7.47 (m, 1H), 7.17-7.12 (m, 1H), 7.03-6.97 (m, 1H), 4.56-4.45 (m, 1H), 4.34-4.24 (m, 1H), 4.13 (br d, J=4.0 Hz, 1H), 4.18-4.07 (m, 1H), 3.95 (br s, 4H), 2.55-2.51 (m, 3H), 2.42-2.20 (m, 2H), 1.47-1.40 (m, 9H).

### Step 2: Methyl 5-(azetidin-2-ylmethoxy)-2-methylbenzoate (81A-2)

To a solution of tert-butyl 2-((3-(methoxycarbonyl)-4-methylphenoxy)methyl)azetidine-1-carboxylate (800 mg, 2.39 mmol, 1.0 eq) in DCM (5.0 mL) was added TFA (3.26 g, 28.6 mmol, 2.12 mL, 12 eq). The mixture was stirred at 20° C. for 1 h. LCMS indicated that the starting material was completely consumed, and the desired mass was detected. The reaction mixture was concentrated under vacuum at 30° C. to give the crude product methyl 5-(azetidin-2-ylmethoxy)-2-methylbenzoate (800 mg, TFA salt) as a yellow oil, which was used in the next step without any further purification as a yellow oil. M+H⁺=236.1 (LCMS).

### Step 3: Methyl 2-methyl-5-((1-methylazetidin-2-yl)methoxy)benzoate (81A-3)

To a solution of (methyl 5-(azetidin-2-ylmethoxy)-2-methylbenzoate (400 mg, 1.03 mmol, 1.0 eq, TFA salt) in MeOH (8.0 mL) was added TEA (80.0 μL), followed by formaldehyde (335 mg, 4.12 mmol, 307 μL, 37% purity in water, 4.0 eq). The resulting mixture was adjusted to pH 6 with a small amount of AcOH. The mixture was stirred at 20° C. for 30 min, then NaBH₃CN (518 mg, 8.25 mmol, 8.0 eq) was added. The resulting reaction mixture was stirred at 20° C. for another 16 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give the crude product methyl 2-methyl-5-((1-methylazetidin-2-yl)methoxy)benzoate (400 mg) as a yellow oil, which was used in the next step without any further purification. M+H⁺=250.1 (LCMS).

### Step 4: 2-Methyl-5-((1-methylazetidin-2-yl)methoxy)benzoic acid (81A-4)

A solution of methyl 2-methyl-5-((1-methylazetidin-2-yl)methoxy)benzoate (300 mg, 1.20 mmol, 1.0 eq) in HCl (2 M aqueous, 9.00 mL) was stirred at 100° C. for 16 h. LCMS indicated that the starting material was completely consumed, and the desired mass was detected. The reaction mixture was allowed to cool to room temperature, treated with H₂O (10 mL) and washed with MTBE (20 mL×2). The aqueous was acidified to pH 6 with NaOH (2 M aqueous). The product was extracted with DCM (10 mL×5) and the combined organic layers were dried over Na₂SO₄, filtered, and concentrated in vacuum to give the crude product 2-methyl-5-((1-methylazetidin-2-yl)methoxy)benzoic acid (220 mg) as a white solid, which was used in the next step without any further purification. M+H⁺=236.1 (LCMS).

### Step 5: 2-Methyl-5-((1-methylazetidin-2-yl)methoxy)-N-(3-(naphthalen-1-yl)oxetan-3-yl)benzamide (Compound 387)

To a solution of 2-methyl-5-[(1-methylazetidin-2-yl)methoxy]benzoic acid (50.0 mg, 212 μmol, 1.0 eq) in DMF (2.0 mL) was added 3-(1-naphthyl)oxetan-3-amine (42.3 mg, 213 μmol, 1.0 eq), followed by HATU (88.9 mg, 234 μmol, 1.1 eq) and DIEA (82.4 mg, 638 μmol, 111 μL, 3.0 eq). The mixture was stirred at 20° C. for 16 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by preparative HPLC (Phenomenex Gemini C18 column (150×40 mm, 10 μm); flow rate: 60 mL/min; gradient: 20%-50% B over 8 min; mobile phase A: 10 mM aqueous NH₄HCO₃, mobile phase B: acetonitrile). 2-Methyl-5-((1-methylazetidin-2-yl)methoxy)-N-(3-(naphthalen-1-yl)oxetan-3-yl)benzamide (23.6 mg, 56.3 μmol, 27% yield) was obtained as a yellow solid. M+H⁺=417.1 (LCMS); ¹H NMR (400 MHZ, DMSO-d₆) δ 9.69 (s, 1H), 8.01-7.95 (m, 1H), 7.88 (d, J=8.0 Hz, 1H), 7.78 (br d, J=6.8 Hz, 2H), 7.57-7.47 (m, 3H), 7.04 (d, J=8.4 Hz, 1H), 6.86 (dd, J=2.8, 8.4 Hz, 1H), 6.66 (d, J=2.8 Hz, 1H), 5.30-5.18 (m, 4H), 3.86 (d, J=5.4 Hz, 2H), 3.28-3.17 (m, 2H), 2.75-2.68 (m, 1H), 2.21 (s, 3H), 1.94 (s, 4H), 1.89-1.79 (m, 1H).

### Example 82: (S)-2-Methyl-5-(2-(methylamino)ethoxy)-N-(2,2,2-trifluoro-1-(naphthalen-1-yl)ethyl)benzamide (Compound 349)

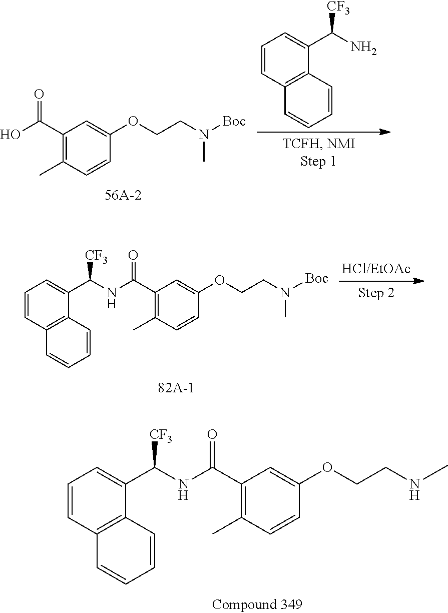 [SMILES: Cc1ccc(OCCN(C)C(=O)OC(C)(C)C)cc1C(=O)O ; N[C@@H](c1cccc2ccccc12)C(F)(F)F ; Cc1ccc(OCCN(C)C(=O)OC(C)(C)C)cc1C(=O)N[C@@H](c1cccc2ccccc12)C(F)(F)F ; CNCCOc1ccc(C)c(C(=O)N[C@@H](c2cccc3ccccc23)C(F)(F)F)c1]

### Step 1: (S)-tert-Butyl methyl(2-(4-methyl-3-((2,2,2-trifluoro-1-(naphthalen-1-yl)ethyl)carbamoyl)phenoxy)ethyl)carbamate (82A-1)

To a solution of 5-(2-((tert-butoxycarbonyl)(methyl)amino)ethoxy)-2-methylbenzoic acid (100 mg, 323 μmol, 1.0 eq), (S)-2,2,2-trifluoro-1-(naphthalen-1-yl)ethanamine (72.8 mg, 323 μmol, 1.0 eq) in ACN (5.0 mL) were added TCFH (109 mg, 388 μmol, 1.2 eq) and 1-methylimidazole (92.9 mg, 1.13 mmol, 90.2 μL, 3.5 eq). The mixture was stirred at 20° C. for 16 h. LCMS indicated that the starting material was completely consumed, and the desired mass was detected. The reaction mixture was poured into H₂O (6.0 mL) and extracted with EtOAc (2.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. (S)-tert-Butyl methyl(2-(4-methyl-3-((2,2,2-trifluoro-1-(naphthalen-1-yl)ethyl)carbamoyl)phenoxy)ethyl) carbamate (90.0 mg, 174 μmol, 54% yield) was obtained as a colorless oil.

### Step 2: (S)-2-Methyl-5-(2-(methylamino)ethoxy)-N-(2,2,2-trifluoro-1-(naphthalen-1-yl)ethyl)benzamide (Compound 349)

To a solution of(S)-tert-butyl methyl(2-(4-methyl-3-((2,2,2-trifluoro-1-(naphthalen-1-yl)ethyl)carbamoyl)phenoxy)ethyl)carbamate (90.0 mg, 174 μmol, 1.0 eq) in EtOAc (1.0 mL) was added HCl/EtOAc (4 M, 1.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 10%-60% B over 8 min; mobile phase A: 0.04% aqueous HCl, mobile phase B: acetonitrile). (S)-2-Methyl-5-(2-(methylamino)ethoxy)-N-(2,2,2-trifluoro-1-(naphthalen-1-yl)ethyl)benzamide (43.6 mg, 96.2 μmol, 55% yield, HCl salt) was obtained as a white solid. M+H⁺=417.0 (LCMS); ¹H NMR (400 MHZ, DMSO-d₆) δ 9.82 (d, J=9.3 Hz, 1H), 8.98 (br s, 2H), 8.28 (d, J=8.5 Hz, 1H), 8.03 (d, J=8.3 Hz, 2H), 7.93 (d, J=7.3 Hz, 1H), 7.72-7.66 (m, 1H), 7.65-7.58 (m, 2H), 7.21 (d, J=8.6 Hz, 1H), 7.01 (dd, J=2.7, 8.4 Hz, 1H), 6.90-6.79 (m, 2H), 4.22 (t, J=5.0 Hz, 2H), 3.28 (t, J=4.9 Hz, 2H), 2.59 (s, 3H), 2.20 (s, 3H).

### Example 83: 5-((1-Ethylazetidin-2-yl)methoxy)-2-methyl-N-(1-(naphthalen-1-yl)cyclopropyl)benzamide (Compound 314)

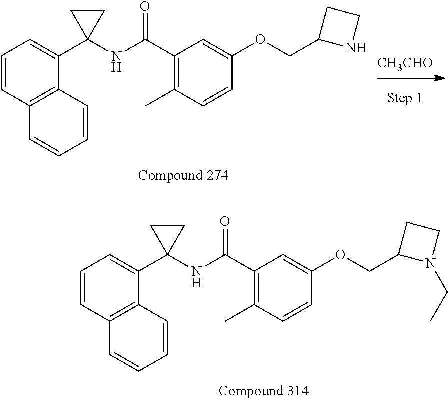

### Step 1: 5-((1-Ethylazetidin-2-yl)methoxy)-2-methyl-N-(1-(naphthalen-1-yl)cyclopropyl)benzamide (Compound 314)

To a solution of 5-(azetidin-2-ylmethoxy)-2-methyl-N-(1-(naphthalen-1-yl)cyclopropyl)benzamide (100 mg, 259 μmol, 1.0 eq, TFA salt) in MeOH (5.0 mL) was added TEA (10.0 μL), followed by the addition of acetaldehyde (57.0 mg, 517 μmol, 72.6 μL, 40% purity in water, 2.0 eq). The resulting mixture was adjusted to pH 6 with a small amount of AcOH. The mixture was stirred at 20° C. for 30 min, then NaBH₃CN (32.5 mg, 517 μmol, 2.0 eq) was added. The resulting mixture was stirred at 20° C. for another 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified preparative HPLC (Phenomenex Luna C18 column (75×30 mm, 3 μm); flow rate: 25 mL/min; gradient: 15%-35% B over 8 min; mobile phase A: 0.2% aqueous FA, mobile phase B: acetonitrile). 5-((1-Ethylazetidin-2-yl)methoxy)-2-methyl-N-(1-(naphthalen-1-yl)cyclopropyl)benzamide (23.7 mg, 51.5 μmol, 35% yield, FA salt) was obtained as a white solid. M+H⁺=415.1 (LCMS); ¹H NMR (400 MHZ, DMSO-d₆) δ=9.10 (s, 1H), 8.63 (d, J=8.3 Hz, 1H), 8.18 (s, 1H), 7.92 (d, J=7.7 Hz, 1H), 7.81 (dd, J=7.8, 10.1 Hz, 2H), 7.60-7.40 (m, 3H), 7.02 (d, J=8.4 Hz, 1H), 6.82 (dd, J=2.8, 8.4 Hz, 1H), 6.59 (d, J=2.7 Hz, 1H), 3.98-3.85 (m, 2H), 3.61-3.55 (m, 1H), 3.39 (dt, J=2.4, 8.0 Hz, 1H), 2.92 (br d, J=8.1 Hz, 1H), 2.72-2.66 (m, 1H), 2.46-2.39 (m, 1H), 2.09-1.99 (m, 1H), 1.94 (s, 4H), 1.34 (br s, 2H), 1.16 (br s, 2H), 0.87 (t, J=7.2 Hz, 3H).

### Example 84: 2-Methyl-N-(1-(naphthalen-1-yl)cyclopropyl)-5-((1-(2,2,2-trifluoroethyl)azetidin-2-yl)methoxy)benzamide (Compound 396)

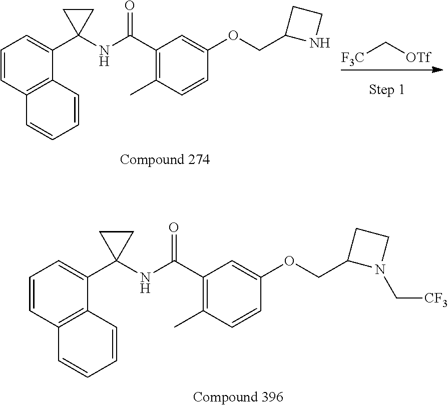 [SMILES: Cc1ccc(OCC2CCN2)cc1C(=O)NC1(c2cccc3ccccc23)CC1 ; O=S(=O)(OCC(F)(F)F)C(F)(F)F ; Cc1ccc(OCC2CCN2CC(F)(F)F)cc1C(=O)NC1(c2cccc3ccccc23)CC1]

### Step 1: 2-Methyl-N-(1-(naphthalen-1-yl)cyclopropyl)-5-((1-(2,2,2-trifluoroethyl)azetidin-2-yl)methoxy)benzamide (Compound 396)

To a solution of 5-(azetidin-2-ylmethoxy)-2-methyl-N-(1-(naphthalen-1-yl)cyclopropyl)benzamide (100 mg, 259 μmol, 1.0 eq) and 2,2,2-trifluoroethyl trifluoromethanesulfonate (60.0 mg, 259 μmol, 1.0 eq) in DMF (10 mL) was added TEA (157 mg, 1.55 mmol, 216 μL, 6.0 eq). The mixture was stirred at 20° C. for 16 h. LCMS indicated that the starting material was completely consumed, and desired mass was detected. The reaction mixture was allowed to cool to room temperature, poured into H₂O (10 mL) and extracted with EtOAc (4.0 mL×3). The combined organic layers were dried over Na₂SO₄, filtered, and concentrated under vacuum to give a residue which was purified preparative HPLC (Phenomenex C18 column (80×40 mm, 3 μm); flow rate: 25 mL/min; gradient: 45%-75% B over 8 min; mobile phase A: 10 mM aqueous NH₄HCO₃, mobile phase B: acetonitrile). 2-Methyl-N-(1-(naphthalen-1-yl)cyclopropyl)-5-((1-(2,2,2-trifluoroethyl)azetidin-2-yl)methoxy)benzamide (14.2 mg, 29.9 μmol, 12% yield) was obtained as a white solid. M+H⁺=469.1 (LCMS); ¹H NMR (400 MHZ, DMSO-d₆) δ 9.09 (s, 1H), 8.65 (d, J=8.1 Hz, 1H), 7.93 (d, J=8.3 Hz, 1H), 7.82 (t, J=8.3 Hz, 2H), 7.63-7.40 (m, 3H), 7.02 (d, J=8.6 Hz, 1H), 6.83 (dd, J=2.7, 8.4 Hz, 1H), 6.61 (d, J=2.6 Hz, 1H), 3.89 (d, J=5.4 Hz, 2H), 3.73-3.62 (m, 1H), 3.44-3.35 (m, 2H), 3.22-3.04 (m, 2H), 2.10-1.96 (m, 2H), 1.94 (s, 3H), 1.35 (br s, 2H), 1.17 (br s, 2H).

### Example 85: 2-Methyl-N-(1-(naphthalen-1-yl)cyclopropyl)-5-((1-phenylazetidin-2-yl)methoxy)benzamide (Compound 331)

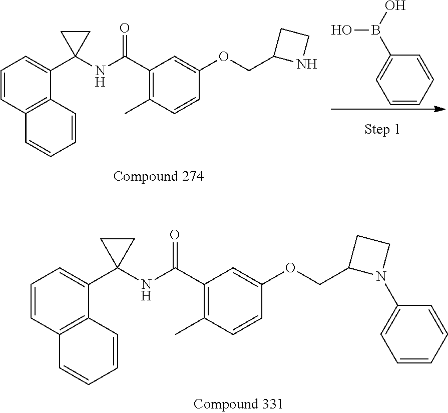

### Step 1: 2-Methyl-N-(1-(naphthalen-1-yl)cyclopropyl)-5-((1-phenylazetidin-2-yl)methoxy)benzamide (Compound 331)

To a mixture of 5-(azetidin-2-ylmethoxy)-2-methyl-N-(1-(naphthalen-1-yl)cyclopropyl)benzamide (150 mg, 300 μmol, 1.0 eq, TFA salt), phenylboronic acid (43.9 mg, 360 μmol, 1.2 eq) in DCE (10 mL) were added Cu(OAc) 2 (60.0 mg, 330 μmol, 1.1 eq), TEA (121 mg, 1.20 mmol, 167 μL, 4.0 eq), and 4 Å molecular sieve (70.0 mg, 1.20 mmol, 4.0 eq). The mixture was degassed and purged with O₂ three times and then it was stirred at 80° C. for 16 h under an O₂ atmosphere. LCMS indicated that the starting material was completely consumed, and the desired mass was detected. The reaction mixture was allowed to cool to room temperature, poured into H₂O (10 mL) and extracted with EtOAc (4.0 mL×3). The combined organic layers were dried over Na₂SO₄, filtered, and concentrated under vacuum to give a residue which was purified by preparative HPLC (Phenomenex Luna C18 column (200×40 mm, 3 μm); flow rate: 25 mL/min; gradient: 65%-98% B over 8 min; mobile phase A: 0.2% aqueous FA, mobile phase B: acetonitrile) to give 2-methyl-N-(1-(naphthalen-1-yl)cyclopropyl)-5-((1-phenylazetidin-2-yl)methoxy)benzamide (140 mg, 334 μmol, 77% yield) as a white solid. M+H⁺=463.1 (LCMS); ¹H NMR (400 MHZ, DMSO-d₆) δ 9.09 (s, 1H), 8.14 (s, 1H), 7.93 (d, J=7.9 Hz, 1H), 7.82 (t, J=7.4 Hz, 2H), 7.60-7.42 (m, 3H), 7.13 (t, J=7.9 Hz, 2H), 7.04 (d, J=8.4 Hz, 1H), 6.89 (dd, J=2.6, 8.3 Hz, 1H), 6.73-6.64 (m, 2H), 6.60 (d, J=7.9 Hz, 2H), 4.26 (br t, J=6.2 Hz, 1H), 4.12 (d, J=5.0 Hz, 2H), 3.93-3.86 (m, 1H), 3.55 (q, J=7.8 Hz, 1H), 2.40-2.30 (m, 1H), 2.29-2.18 (m, 1H), 1.97 (s, 3H), 1.36 (br s, 2H), 1.17 (br s, 2H).

### Example 86: 5-((1-Benzylazetidin-2-yl)methoxy)-2-methyl-N-(1-(naphthalen-1-yl)cyclopropyl)benzamide (Compound 330)

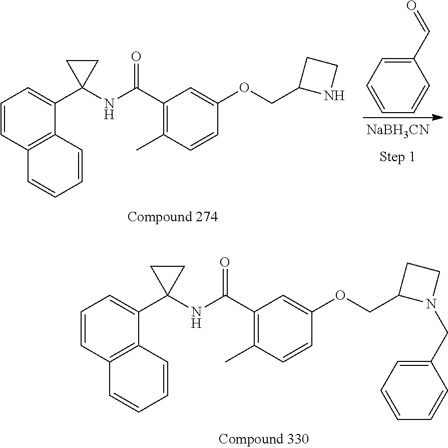

### Step 1: 5-((1-Benzylazetidin-2-yl)methoxy)-2-methyl-N-(1-(naphthalen-1-yl)cyclopropyl)benzamide (Compound 330)

To a solution of 5-(azetidin-2-ylmethoxy)-2-methyl-N-(1-(naphthalen-1-yl)cyclopropyl)benzamide (100 mg, 200 μmol, 1.0 eq, TFA salt) in MeOH (5.0 mL) was added TEA (10.0 μL), followed by the addition of benzaldehyde (21.2 mg, 200 μmol, 158 μL, 2.0 eq). The resulting mixture was adjusted to pH 6 with a small amount of AcOH. The mixture was stirred at 20° C. for 30 min, then NaBH₃CN (134 mg, 2.13 mmol, 2.0 eq) was added. The reaction mixture was stirred at 20° C. for another 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex C18 column (80×40 mm, 3 μm); flow rate: 25 mL/min; gradient: 35%-65% B over 8 min; mobile phase A: 10 mM aqueous NH₄HCO₃, mobile phase B: acetonitrile). 5-((1-Benzylazetidin-2-yl)methoxy)-2-methyl-N-(1-(naphthalen-1-yl)cyclopropyl)benzamide (33.0 mg, 69.2 μmol, 35% yield) was obtained as a white solid. M+H⁺=477.1 (LCMS); ¹H NMR (400 MHZ, DMSO-d₆) δ 9.06 (s, 1H), 8.66 (br d, J=8.4 Hz, 1H), 7.92 (br d, J=7.8 Hz, 1H), 7.82 (br t, J=6.4 Hz, 2H), 7.59-7.43 (m, 3H), 7.25-7.18 (m, 4H), 7.18-7.11 (m, 1H), 7.00 (d, J=8.4 Hz, 1H), 6.76 (dd, J=2.5, 8.3 Hz, 1H), 6.54 (d, J=2.5 Hz, 1H), 3.87-3.71 (m, 3H), 3.54-3.40 (m, 2H), 3.17 (br t, J=6.2 Hz, 1H), 2.79 (q, J=7.9 Hz, 1H), 2.01 (br d, J=8.4 Hz, 1H), 1.95 (s, 3H), 1.92-1.83 (m, 1H), 1.35 (br s, 2H), 1.17 (br s, 2H).

### Example 87: 5-((1-Isopropylazetidin-2-yl)methoxy)-2-methyl-N-(1-(naphthalen-1-yl)cyclopropyl)benzamide (Compound 323)

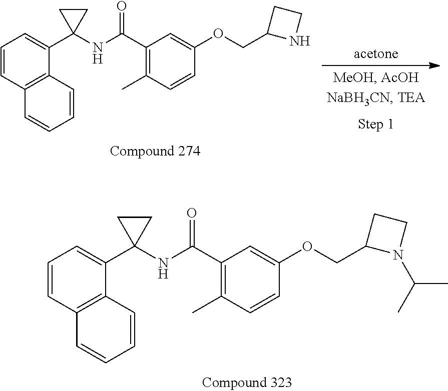 [SMILES: Cc1ccc(OCC2CCN2)cc1C(=O)NC1(c2cccc3ccccc23)CC1 ; "acetone" ; Cc1ccc(OCC2CCN2C(C)C)cc1C(=O)NC1(c2cccc3ccccc23)CC1]

### Step 1: 5-((1-Isopropylazetidin-2-yl)methoxy)-2-methyl-N-(1-(naphthalen-1-yl)cyclopropyl)benzamide (Compound 323)

To a solution of 5-(azetidin-2-ylmethoxy)-2-methyl-N-(1-(naphthalen-1-yl)cyclopropyl)benzamide (100 mg, 99.9 μmol, 50% purity, 1.0 eq, TFA salt) in MeOH (5.0 mL) was added TEA (10.0 μL), followed by the addition of acetone (11.6 mg, 200 μmol, 14.7 μL, 2.0 eq). The resulting mixture was adjusted to pH 6 with a small amount of AcOH. The mixture was stirred at 20° C. for 30 min, then NaBH₃CN (12.6 mg, 200 μmol, 2.0 eq) was added. The reaction mixture was stirred at 20° C. for another 16 h. LCMS indicated that the starting material was completely consumed, and the desired mass was detected. The reaction mixture was poured into H₂O (4.0 mL) and extracted with DCM (3.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 30%-35% B over 8 min; mobile phase A: 0.2% aqueous FA, mobile phase B: acetonitrile). 5-((1-Isopropylazetidin-2-yl)methoxy)-2-methyl-N-(1-(naphthalen-1-yl)cyclopropyl)benzamide (18.5 mg, 43.1 μmol, 43% yield) was obtained as a white solid. M+H⁺=429.1 (LCMS); ¹H NMR (400 MHZ, CDCl₃) δ 8.54 (d, J=8.4 Hz, 1H), 7.96 (dd, J=0.9, 7.0 Hz, 1H), 7.89 (d, J=8.1 Hz, 1H), 7.80 (d, J=8.3 Hz, 1H), 7.63-7.55 (m, 1H), 7.54-7.44 (m, 2H), 7.02-6.97 (m, 1H), 6.95-6.84 (m, 1H), 6.73 (s, 2H), 4.47-4.28 (m, 1H), 4.17-3.74 (m, 3H), 3.45-3.29 (m, 1H), 3.04-2.88 (m, 1H), 2.31 (br d, J=2.1 Hz, 2H), 2.12 (s, 3H), 1.62-1.53 (m, 2H), 1.45-1.35 (m, 2H), 1.29-1.13 (m, 6H).

### Example 88: 5-((1-(Cyclopropylmethyl)azetidin-2-yl)methoxy)-2-methyl-N-(1-(naphthalen-1-yl)cyclopropyl)benzamide (Compound 332)

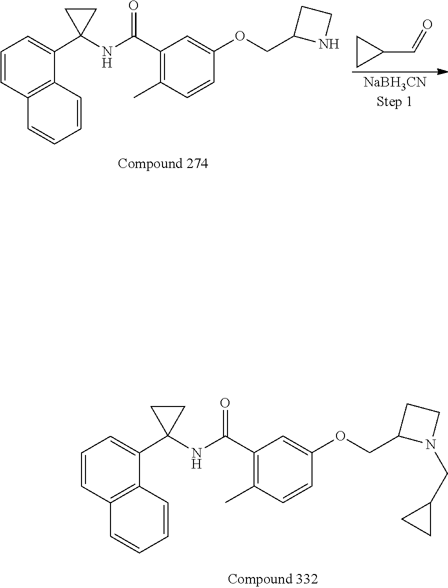

### Step 1: 5-((1-(Cyclopropylmethyl)azetidin-2-yl)methoxy)-2-methyl-N-(1-(naphthalen-1-yl)cyclopropyl)benzamide (Compound 332)

To a solution of 5-(azetidin-2-ylmethoxy)-2-methyl-N-(1-(naphthalen-1-yl)cyclopropyl)benzamide (100 mg, 200 μmol, 1.0 eq) in MeOH (3.0 mL) was added TEA (10.0 μL), followed by the addition of cyclopropanecarbaldehyde (28.0 mg, 400 μmol, 30.0 μL, 2.0 eq). The resulting mixture was adjusted to pH 6 with a small amount of AcOH. The mixture was stirred at 20° C. for 30 min, and then NaBH₃CN (25.1 mg, 400 μmol, 2.0 eq) was added. The reaction mixture was stirred at 20° C. for another 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 30%-60% B over 8 min; mobile phase A: 0.2% aqueous FA, mobile phase B: acetonitrile). 5-((1-(Cyclopropylmethyl)azetidin-2-yl)methoxy)-2-methyl-N-(1-(naphthalene-1-yl)cyclopropyl)benzamide (13.3 mg, 30.2 μmol, 15% yield, FA salt) was obtained as a white solid. M+H⁺=441.1 (LCMS); ¹H NMR (400 MHZ, DMSO-d₆) δ 9.79 (m, 1H), 9.10 (s, 1H), 8.64 (d, J=8.4 Hz, 1H), 7.94 (d, J=7.8 Hz, 1H), 7.83 (t, J=7.9 Hz, 2H), 7.61-7.40 (m, 3H), 7.08 (d, J=8.3 Hz, 1H), 6.90 (dd, J=2.6, 8.4 Hz, 1H), 6.68 (d, J=2.6 Hz, 1H), 4.70 (br s, 1H), 4.33-4.15 (m, 2H), 4.06-3.89 (m, 2H), 3.23-3.12 (m, 1H), 3.02-2.87 (m, 1H), 2.43-2.25 (m, 2H), 1.97 (s, 3H), 1.35 (br s, 2H), 1.19 (br s, 2H), 0.95 (br s, 1H), 0.52 (td, J=4.3, 8.6 Hz, 2H), 0.41-0.19 (m, 2H).

### Example 89: 5-((1-(2-Methoxyethyl)azetidin-2-yl)methoxy)-2-methyl-N-(1-(naphthalen-1-yl)cyclopropyl)benzamide (Compound 348)

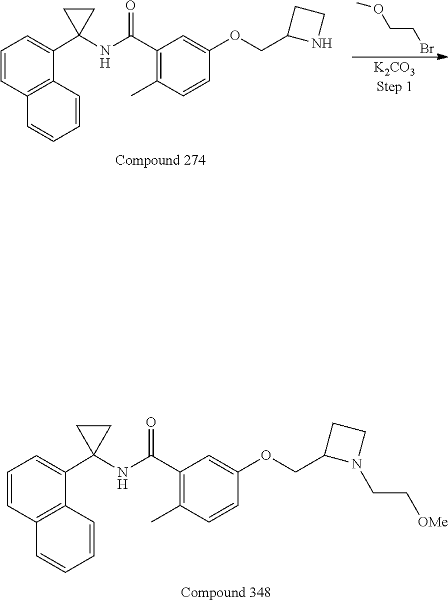

### Step 1: 5-((1-(2-Methoxyethyl)azetidin-2-yl)methoxy)-2-methyl-N-(1-(naphthalen-1-yl)cyclopropyl)benzamide (Compound 348)

To a solution of 5-(azetidin-2-ylmethoxy)-2-methyl-N-(1-(naphthalen-1-yl)cyclopropyl)benzamide (150 mg, 388 μmol, 1.0 eq) in ACN (6.0 mL) was added K₂CO₃ (161 mg, 1.16 mmol, 3.0 eq). The mixture was stirred at 20° C. for 30 min, then 1-bromo-2-methoxyethane (80.9 mg, 582 μmol, 54.7 μL, 1.5 eq) was added. The mixture was stirred at 80° C. for 16 h. LCMS indicated that the starting material was completely consumed, and the desired mass was detected. The reaction mixture was allowed to cool to room temperature, poured into H₂O (6.0 mL) and extracted with EtOAc (2.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 1%-45% B over 8 min; mobile phase A: 0.04% aqueous HCl, mobile phase B: acetonitrile). 5-((1-(2-Methoxyethyl)azetidin-2-yl)methoxy)-2-methyl-N-(1-(naphthalen-1-yl)cyclopropyl)benzamide (27.4 mg, 54.8 μmol, 14% yield, HCl salt) was obtained as a white solid. M+H⁺=445.1 (LCMS); ¹H NMR (400 MHz, DMSO-d₆) δ 10.53 (br d, J=3.8 Hz, 1H), 9.15 (s, 1H), 8.66 (d, J=8.4 Hz, 1H), 7.93 (d, J=7.8 Hz, 1H), 7.82 (t, J=7.2 Hz, 2H), 7.60-7.43 (m, 3H), 7.08 (d, J=8.4 Hz, 1H), 6.89 (dd, J=2.5, 8.4 Hz, 1H), 6.68 (d, J=2.4 Hz, 1H), 4.78-4.63 (m, 1H), 4.38 (dd, J=8.3, 11.1 Hz, 1H), 4.16 (dd, J=3.2, 11.2 Hz, 1H), 3.96-3.89 (m, 2H), 3.62-3.55 (m, 1H), 3.54-3.46 (m, 1H), 3.43-3.32 (m, 2H), 3.18 (s, 3H), 2.42-2.22 (m, 2H), 1.96 (s, 3H), 1.36 (br s, 2H), 1.18 (br s, 2H).

### Example 90: 5-((1-(2-Hydroxyethyl)azetidin-2-yl)methoxy)-2-methyl-N-(1-(naphthalen-1-yl)cyclopropyl)benzamide (Compound 341)

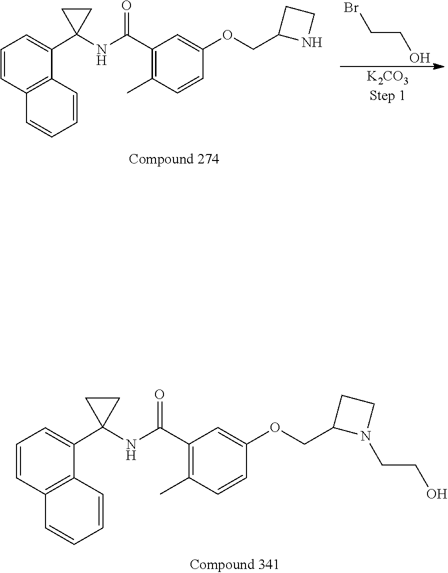

### Step 1: 5-((1-(2-Hydroxyethyl)azetidin-2-yl)methoxy)-2-methyl-N-(1-(naphthalen-1-yl)cyclopropyl)benzamide (Compound 341)

To a solution of 5-(azetidin-2-ylmethoxy)-2-methyl-N-(1-(naphthalen-1-yl)cyclopropyl)benzamide (200 mg, 517 μmol, 1.0 eq) and 2-bromoethanol (64.7 mg, 517 μmol, 36.7 μL, 1.0 eq) in ACN (5.0 mL) was added K₂CO₃ (215 mg, 1.55 mmol, 3.0 eq). The mixture was stirred at 80° C. for 16 h. LCMS indicated that the starting material was completely consumed, and the desired mass was detected. The reaction mixture was allowed to cool to room temperature, poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 30%-70% B over 8 min; mobile phase A: 0.2% aqueous FA, mobile phase B: acetonitrile). 5-((1-(2-Hydroxyethyl)azetidin-2-yl)methoxy)-2-methyl-N-(1-(naphthalen-1-yl)cyclopropyl)benzamide (32.9 mg, 68.5 μmol, 13% yield, FA salt) was obtained as a white solid. M+H⁺=431.1 (LCMS); ¹H NMR (400 MHZ, DMSO-d₆) δ 9.08 (s, 1H), 8.67 (d, J=8.4 Hz, 1H), 8.20 (s, 1H), 7.94 (d, J=8.0 Hz, 1H), 7.87-7.79 (m, 2H), 7.62-7.42 (m, 3H), 7.03 (d, J=8.4 Hz, 1H), 6.83 (dd, J=2.7, 8.3 Hz, 1H), 6.61 (d, J=2.6 Hz, 1H), 3.95-3.84 (m, 2H), 3.44 (br d, J=3.4 Hz, 1H), 3.39-3.29 (m, 3H), 2.88 (br d, J=8.1 Hz, 1H), 2.69 (td, J=6.1, 11.9 Hz, 1H), 2.45 (br dd, J=6.1, 11.8 Hz, 1H), 2.06-1.85 (m, 5H), 1.37 (s, 2H), 1.22-1.11 (m, 2H).

### Example 91: 2-Methyl-5-((2-methylazetidin-2-yl)methoxy)-N-(1-(naphthalen-1-yl)cyclopropyl)benzamide (Compound 394)

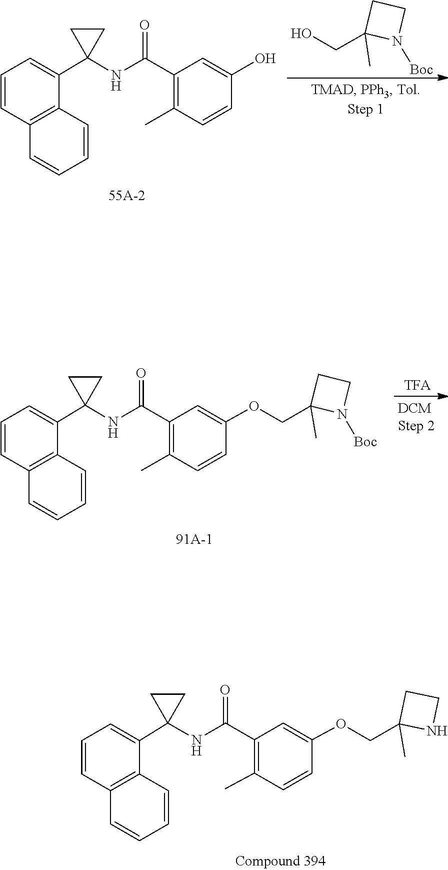

### Step 1: tert-Butyl 2-methyl-2-((4-methyl-3-((1-(naphthalen-1-yl)cyclopropyl)carbamoyl) phenoxy)methyl)azetidine-1-carboxylate (91A-1)

To a solution of 5-hydroxy-2-methyl-N-(1-(naphthalen-1-yl)cyclopropyl)benzamide (300 mg, 945 μmol, 1.0 eq) and tert-butyl 2-(hydroxymethyl)-2-methylazetidine-1-carboxylate (190 mg, 945 μmol, 1.0 eq) in toluene (18 mL) were added TMAD (488 mg, 2.84 mmol, 3.0 eq), PPh₃ (744 mg, 2.84 mmol, 3.0 eq). The mixture was degassed and purged with N₂ three times and stirred at 100° C. for 16 h under a N₂ atmosphere. LCMS indicated that the starting material was completely consumed, and the desired mass was detected. The reaction mixture was allowed to cool to room temperature, poured into H₂O (15 mL) and extracted with EtOAc (8.0 mL×3). The combined organic layers were dried over Na₂SO₄, filtered, and concentrated under vacuum to give a residue which was purified by flash silica gel chromatography using a gradient of EtOAc/petroleum ether from 1/100 to 3/5. tert-Butyl 2-methyl-2-((4-methyl-3-((1-(naphthalen-1-yl)cyclopropyl)carbamoyl)phenoxy)methyl)azetidine-1-carboxylate (370 mg, 246 μmol, 26% yield) was obtained as a yellow solid. M+H⁺=501.3 (LCMS); ¹H NMR (400 MHz, CDCl₃) δ 8.53 (br s, 1H), 8.00-7.93 (m, 1H), 7.90 (br d, J=8.1 Hz, 1H), 7.79 (s, 1H), 7.62-7.54 (m, 1H), 7.54-7.44 (m, 2H), 7.05-6.94 (m, 1H), 6.85-6.62 (m, 3H), 4.13-4.02 (m, 1H), 3.90-3.67 (m, 3H), 2.44-2.31 (m, 1H), 2.19-2.08 (m, 3H), 1.99-1.89 (m, 1H), 1.58 (br d, J=17.9 Hz, 3H), 1.52-1.40 (m, 4H), 1.36-1.22 (m, 9H).

### Step 2: 2-Methyl-5-((2-methylazetidin-2-yl)methoxy)-N-(1-(naphthalen-1-yl)cyclopropyl)benzamide (Compound 394)

To a stirred solution of tert-butyl 2-methyl-2-((4-methyl-3-((1-(naphthalen-1-yl)cyclopropyl) carbamoyl)phenoxy)methyl)azetidine-1-carboxylate (180 mg, 360 μmol, 1.0 eq) in DCM (9.0 mL) was added TFA (3.6 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna (80×30 mm, 3 μm); flow rate: 25 mL/min; gradient: 1%-40% B over 8 min; mobile phase A: 0.04% aqueous HCl, mobile phase B: acetonitrile). 2-Methyl-5-((2-methylazetidin-2-yl)methoxy)-N-(1-(naphthalen-1-yl)cyclopropyl)benzamide (13.5 mg, 25.9 μmol, 7% yield, HCl salt) was obtained as a brown solid. M+H⁺=401.1 (LCMS); 1H NMR (400 MHZ, DMSO-d₆) δ 9.10 (s, 1H), 9.02-8.85 (m, 1H), 8.82-8.58 (m, 2H), 7.97-7.90 (m, 1H), 7.87-7.80 (m, 2H), 7.61-7.42 (m, 3H), 7.14-7.05 (m, 1H), 6.96-6.89 (m, 1H), 6.75-6.69 (m, 1H), 4.21-4.13 (m, 1H), 4.01-3.94 (m, 1H), 3.88-3.73 (m, 2H), 2.46 (br d, J=10.8 Hz, 1H), 2.26 (br s, 1H), 2.01-1.93 (m, 3H), 1.61-1.52 (m, 3H), 1.41-1.33 (m, 2H), 1.22-1.13 (m, 2H).

### Example 92: 5-(2-Hydroxyethoxy)-2-methyl-N-(1-(naphthalen-1-yl)cyclopropyl)benzamide (Compound 393)

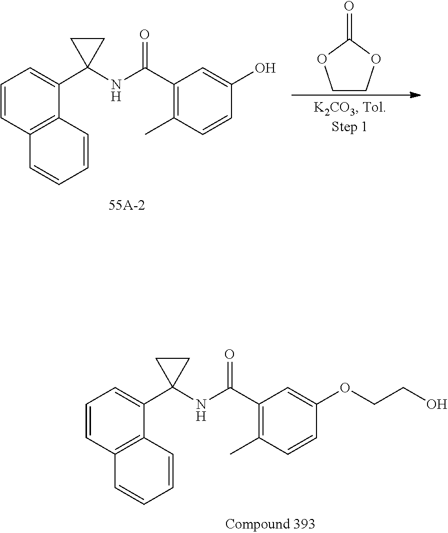 [SMILES: Cc1ccc(O)cc1C(=O)NC1(c2cccc3ccccc23)CC1 ; O=C1OCCO1 ; Cc1ccc(OCCO)cc1C(=O)NC1(c2cccc3ccccc23)CC1]

### Step 1: 5-(2-Hydroxyethoxy)-2-methyl-N-(1-(naphthalen-1-yl)cyclopropyl)benzamide (Compound 393)

To a solution of 5-hydroxy-2-methyl-N-(1-(naphthalen-1-yl)cyclopropyl)benzamide (70.0 mg, 221 μmol, 1.0 eq) and 1,3-dioxolan-2-one (38.9 mg, 441 μmol, 2.0 eq) in toluene (3.5 mL) was added K₂CO₃ (61.0 mg, 441 μmol, 2.0 eq), the mixture was degassed and purged with N₂ three times and stirred at 110° C. for 16 h under a N₂ atmosphere. LCMS indicated that the starting material was completely consumed, and the desired mass was detected. The reaction mixture was allowed to cool to room temperature, poured into H₂O (3.0 mL) and extracted with EtOAc (3.0 mL×3). The combined organic layers were dried over Na₂SO₄, filtered, and concentrated under vacuum to give a residue which was purified by preparative HPLC (Welch Ultimate XB SIO₂ (100×30 mm, 10 μm); flow rate: 40 mL/min; gradient: 5%-95% B over 10 min; mobile phase A: heptane, mobile phase B: EtOH). 5-(2-Hydroxyethoxy)-2-methyl-N-(1-(naphthalen-1-yl)cyclopropyl)benzamide (30.9 mg, 81.9 μmol, 37% yield) was obtained as a brown solid. M+H⁺=362.0 (LCMS); ¹H NMR (400 MHz, DMSO-d₆) δ 9.14-9.02 (m, 1H), 8.70-8.62 (m, 1H), 7.98-7.89 (m, 1H), 7.86-7.78 (m, 2H), 7.61-7.41 (m, 3H), 7.06-6.99 (m, 1H), 6.86-6.79 (m, 1H), 6.63-6.58 (m, 1H), 4.83-4.76 (m, 1H), 3.92-3.84 (m, 2H), 3.69-3.60 (m, 2H), 2.01-1.92 (m, 3H), 1.40-1.31 (m, 2H), 1.21-1.12 (m, 2H).

### Example 93: 5-(2-Amino-2-oxoethoxy)-2-methyl-N-(1-(naphthalen-1-yl)cyclopropyl)benzamide (Compound 285)

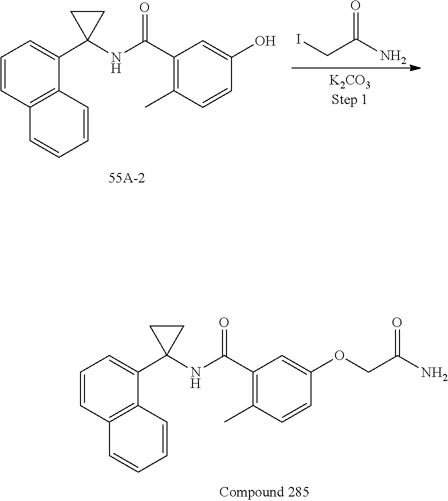

### Step 1: 5-(2-Amino-2-oxoethoxy)-2-methyl-N-(1-(naphthalen-1-yl)cyclopropyl)benzamide (Compound 285)

To a solution of 2-iodoacetamide (61.2 mg, 331 μmol, 1.5 eq) in acetone (10 mL) was added K₂CO₃ (91.5 mg, 662 μmol, 3.0 eq). The mixture was stirred at 20° C. for 30 min. 5-hydroxy-2-methyl-N-(1-(naphthalen-1-yl)cyclopropyl)benzamide (70.0 mg, 221 μmol, 1.0 eq) was added. The mixture was stirred at 90° C. for 16 h. LCMS indicated that the starting material was completely consumed, and the desired mass was detected. The reaction mixture was allowed to cool to room temperature, poured into H₂O (10 mL) and extracted with EtOAc (4.0 mL×2). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 30%-70% B over 8 min; mobile phase A: 0.2% aqueous FA, mobile phase B: acetonitrile). 5-(2-Amino-2-oxoethoxy)-2-methyl-N-(1-(naphthalen-1-yl)cyclopropyl)benzamide (37.2 mg, 99.4 μmol, 45% yield) was obtained as a white solid. M+H⁺=375.0 (LCMS); ¹H NMR (400 MHZ, DMSO-d₆) δ 9.09 (s, 1H), 8.65 (d, J=8.4 Hz, 1H), 7.93 (d, J=8.0 Hz, 1H), 7.82 (dd, J=3.1, 7.6 Hz, 2H), 7.60-7.42 (m, 4H), 7.33 (br s, 1H), 7.04 (d, J=8.4 Hz, 1H), 6.84 (dd, J=2.8, 8.4 Hz, 1H), 6.69 (d, J=2.6 Hz, 1H), 4.34 (s, 2H), 1.95 (s, 3H), 1.35 (s, 2H), 1.17 (s, 2H).

### Example 94: 5-(Azetidin-3-yloxy)-2-methyl-N-(1-(naphthalen-1-yl)cyclopropyl)benzamide (Compound 171)

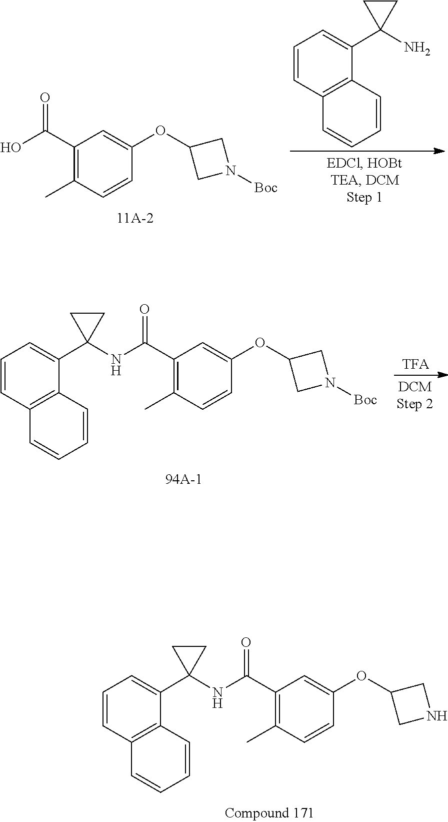 [SMILES: Cc1ccc(OC2CN(C(=O)OC(C)(C)C)C2)cc1C(=O)O ; NC1(c2cccc3ccccc23)CC1 ; Cc1ccc(OC2CN(C(=O)OC(C)(C)C)C2)cc1C(=O)NC1(c2cccc3ccccc23)CC1 ; Cc1ccc(OC2CNC2)cc1C(=O)NC1(c2cccc3ccccc23)CC1]

### Step 1: tert-Butyl 3-(4-methyl-3-((1-(naphthalen-1-yl)cyclopropyl)carbamoyl)phenoxy) azetidine-1-carboxylate (94A-1)

To a solution of 1-(naphthalen-1-yl)cyclopropanamine (59.7 mg, 326 μmol, 1.0 eq) and 5-((1-(tert-butoxycarbonyl)azetidin-3-yl)oxy)-2-methylbenzoic acid (100 mg, 326 μmol, 1.0 eq) in DCM (4.0 mL) were added TEA (98.8 mg, 978 μmol, 136 μL, 3.0 eq), EDCI (62.4 mg, 489 μmol, 1.5 eq) and HOBt (43.8 mg, 489 μmol, 1.5 eq). The mixture was stirred at 20° C. for 16 h. LCMS indicated that the starting material was completely consumed, and the desired mass was detected. The mixture was poured into H₂O (20 mL) and extracted with DCM (15 mL×8). The combined organic layers were dried over Na₂SO₄, filtered, and concentrated under vacuum to give a residue which was purified by preparative TLC (EtOAc/petroleum ether=1/3, R_{f}=0.4). tert-Butyl 3-(4-methyl-3-((1-(naphthalen-1-yl)cyclopropyl)carbamoyl) phenoxy)azetidine-1-carboxylate (90.0 mg, 190 μmol, 65% yield) was obtained as a brown solid. M+H⁺=473.2 (LCMS).

### Step 2: 5-(Azetidin-3-yloxy)-2-methyl-N-(1-(naphthalen-1-yl)cyclopropyl)benzamide (Compound 171)

To a solution of tert-butyl 3-(4-methyl-3-((1-(naphthalen-1-yl)cyclopropyl)carbamoyl) phenoxy)azetidine-1-carboxylate (80.0 mg, 169 μmol, 1.0 eq) in DCM (4.0 mL) was added TFA (6.16 g, 54.0 mmol, 4.00 mL, 319 eq). The mixture was stirred at 20° C. for 1 h. LCMS indicated that the starting material was completely consumed, and the desired mass was detected. The reaction mixture was concentrated under vacuum at 30° C. to give a residue which was purified by preparative HPLC (Phenomenex Gemini C18 column (80×30 mm, 3 μm); flow rate: 25 mL/min; gradient: 15%-45% B over 8 min; mobile phase A: 0.1% aqueous TFA, mobile phase B: acetonitrile). 5-(Azetidin-3-yloxy)-2-methyl-N-(1-(naphthalen-1-yl)cyclopropyl)benzamide (40.0 mg, 82.2 μmol, 49% yield, TFA salt) was obtained as a white solid. M+H⁺=373.1 (LCMS); ¹H NMR (400 MHZ, CD₃OD) δ 9.24-9.20 (m, 1H), 8.60-8.56 (m, 1H), 7.93-7.89 (m, 2H), 7.84-7.81 (m, 1H), 7.62-7.43 (m, 3H), 7.12-7.08 (m, 1H), 6.80-6.76 (m, 1H), 6.54-6.51 (m, 1H), 5.06-5.00 (m, 1H), 4.46-4.41 (m, 2H), 4.07 (br s, 2H), 2.00 (s, 3H), 1.47-1.43 (m, 2H), 1.33-1.31 (m, 2H).

### Example 95: 5-(3-Aminocyclobutoxy)-2-methyl-N-(1-(naphthalen-1-yl)cyclopropyl)benzamide (Compound 265)

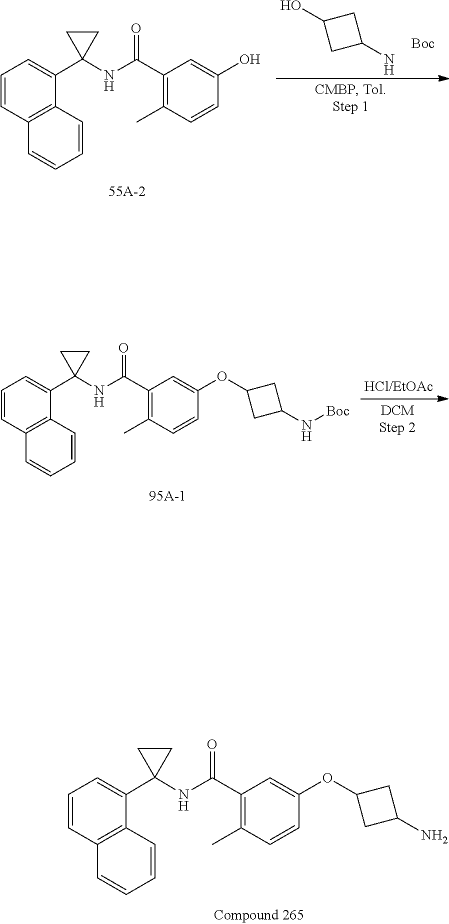 [SMILES: Cc1ccc(O)cc1C(=O)NC1(c2cccc3ccccc23)CC1 ; NC1CC(O)C1 ; Cc1ccc(OC2CC(NC(=O)OC(C)(C)C)C2)cc1C(=O)NC1(c2cccc3ccccc23)CC1 ; "Boc" ; Cc1ccc(OC2CC(N)C2)cc1C(=O)NC1(c2cccc3ccccc23)CC1]

### Step 1: tert-Butyl(3-(4-methyl-3-((1-(naphthalen-1-yl)cyclopropyl)carbamoyl)phenoxy)cyclobutyl)carbamate (95A-1)

To a solution of 5-hydroxy-2-methyl-N-(1-(naphthalen-1-yl)cyclopropyl)benzamide (200 mg, 630 μmol, 1.0 eq) and tert-butyl(3-hydroxycyclobutyl)carbamate (177 mg, 945 μmol, 1.5 eq) in toluene (3.0 mL) was added CMBP (228 mg, 945 μmol, 1.5 eq) at 25° C. The mixture was stirred at 110° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×3). The combined organic layers were washed with brine (10 mL×3), dried over Na₂SO₄, filtered, and concentrated under vacuum to give the crude product tert-butyl(3-(4-methyl-3-((1-(naphthalen-1-yl)cyclopropyl)carbamoyl)phenoxy)cyclobutyl)carbamate (300 mg), which was used in the next step without any further purification. M+H⁺=431.1 (LCMS).

### Step 2: 5-(3-Aminocyclobutoxy)-2-methyl-N-(1-(naphthalen-1-yl)cyclopropyl)benzamide (Compound 265)

To a mixture of tert-butyl(3-(4-methyl-3-((1-(naphthalen-1-yl)cyclopropyl)carbamoyl) phenoxy)cyclobutyl)carbamate (300 mg, 617 μmol, 1.0 eq) in EtOAc (3.0 mL) was added HCl/EtOAc (4 M, 6.0 mL). The mixture was stirred at 25° C. for 1 h. LCMS indicated that the starting material was completely consumed, and the desired mass was detected. The reaction mixture concentrated under vacuum to give a residue which was purified by preparative HPLC (Phenomenex Luna column (80×30 mm, 3 μm); flow rate: 25 mL/min; gradient: 20%-45% B over 8 min; mobile phase A: 0.04% aqueous HCl, mobile phase B: acetonitrile). 5-(3-Aminocyclobutoxy)-2-methyl-N-(1-(naphthalen-1-yl)cyclopropyl)benzamide (96.3 mg, 249 μmol, 40% yield, HCl salt) was obtained as a white solid. M+H⁺=387.1 (LCMS); ¹H NMR (400 MHZ, DMSO-d₆) δ 9.13 (d, J=4.4 Hz, 1H), 8.65 (d, J=8.3 Hz, 1H), 8.35 (br d, J=10.9 Hz, 3H), 7.93 (d, J=8.2 Hz, 1H), 7.86-7.76 (m, 2H), 7.63-7.56 (m, 1H), 7.56-7.50 (m, 1H), 7.46 (t, J=7.7 Hz, 1H), 7.08-6.98 (m, 1H), 6.77-6.67 (m, 1H), 6.48 (d, J=2.6 Hz, 1H), 4.97-4.40 (m, 1H), 3.77-3.31 (m, 1H), 2.77-2.65 (m, 1H), 2.60-2.52 (m, 1H), 2.38-2.28 (m, 1H), 2.20-2.09 (m, 1H), 1.96 (d, J=3.7 Hz, 3H), 1.35 (br s, 2H), 1.17 (br s, 2H).

### Example 96: 2-Methyl-N-(1-(naphthalen-1-yl)cyclopropyl)-5-(pyrrolidin-2-ylmethoxy)benzamide (Compound 263)

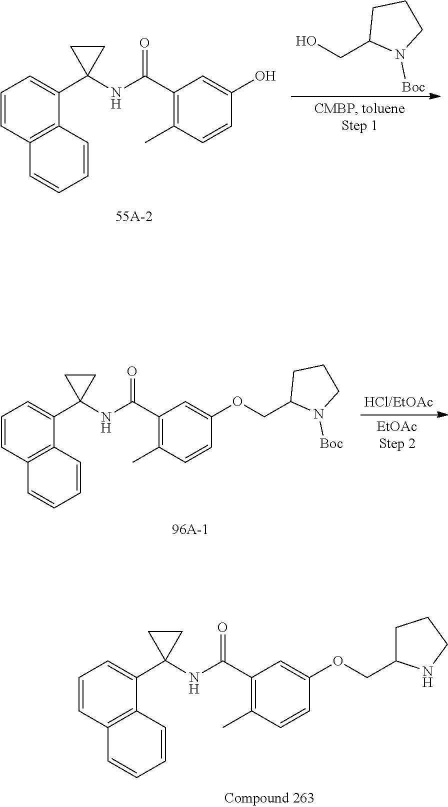 [SMILES: Cc1ccc(O)cc1C(=O)NC1(c2cccc3ccccc23)CC1 ; CC(C)(C)OC(=O)N1CCCC1CO ; Cc1ccc(OCC2CCCN2C(=O)OC(C)(C)C)cc1C(=O)NC1(c2cccc3ccccc23)CC1 ; Cc1ccc(OCC2CCCN2)cc1C(=O)NC1(c2cccc3ccccc23)CC1]

### Step 1: tert-Butyl 2-((4-methyl-3-((1-(naphthalen-1-yl)cyclopropyl)carbamoyl)phenoxy)methyl)pyrrolidine-1-carboxylate (96A-1)

A mixture of 5-hydroxy-2-methyl-N-(1-(naphthalen-1-yl)cyclopropyl)benzamide (150 mg, 473 μmol, 1.0 eq), tert-butyl 2-(hydroxymethyl) pyrrolidine-1-carboxylate (95.1 mg, 473 μmol, 1.0 eq) and CMBP (171 mg, 709 μmol, 1.5 eq) in toluene (8.0 mL) was degassed and purged with N₂ three times. The mixture was stirred at 110° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. tert-Butyl 2-((4-methyl-3-((1-(naphthalen-1-yl)cyclopropyl)carbamoyl)phenoxy)methyl) pyrrolidine-1-carboxylate (200 mg, 400 μmol, 85% yield) was obtained as a yellow oil. M+H⁺=429.1 (LCMS); ¹H NMR (400 MHZ, CDCl₃) δ 8.53-8.46 (m, 1H), 7.98-7.93 (m, 1H), 7.92-7.87 (m, 1H), 7.83-7.77 (m, 1H), 7.60-7.45 (m, 3H), 7.02-6.96 (m, 1H), 6.67 (br s, 3H), 4.09-4.00 (m, 1H), 3.82-3.65 (m, 1H), 3.46-3.28 (m, 2H), 2.12 (s, 3H), 2.01-1.82 (m, 4H), 1.60 (s, 7H), 1.49-1.43 (m, 6H).

### Step 2: 2-Methyl-N-(1-(naphthalen-1-yl)cyclopropyl)-5-(pyrrolidin-2-ylmethoxy)benzamide (Compound 263)

To a stirred solution of tert-butyl 2-((4-methyl-3-((1-(naphthalen-1-yl)cyclopropyl)carbamoyl) phenoxy)methyl) pyrrolidine-1-carboxylate (100 mg, 200 μmol, 1.0 eq) in EtOAc (5.0 mL) was added HCl/EtOAc (4 M, 10 mL) at 0° C. The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 15%-65% B over 8 min; mobile phase A: 0.04% aqueous HCl, mobile phase B: acetonitrile). 2-Methyl-N-(1-(naphthalen-1-yl)cyclopropyl)-5-(pyrrolidin-2-ylmethoxy)benzamide (51.3 mg, 117 μmol, 59% yield, HCl salt) was obtained as a white solid. M+H⁺=401.1 (LCMS); ¹H NMR (400 MHZ, DMSO-d₆) δ 9.53-9.36 (m, 1H), 9.12 (s, 1H), 8.88 (br dd, J=1.7, 3.6 Hz, 1H), 8.66 (d, J=8.3 Hz, 1H), 7.93 (d, J=8.0 Hz, 1H), 7.86-7.80 (m, 2H), 7.61-7.44 (m, 3H), 7.07 (d, J=8.4 Hz, 1H), 6.88 (dd, J=2.6, 8.4 Hz, 1H), 6.67 (d, J=2.6 Hz, 1H), 4.18-4.11 (m, 1H), 4.08-4.00 (m, 1H), 3.82 (br d, J=2.9 Hz, 1H), 3.16 (br s, 2H), 2.14-2.01 (m, 1H), 1.97 (s, 3H), 1.95-1.81 (m, 2H), 1.73-1.63 (m, 1H), 1.36 (s, 2H), 1.21-1.15 (m, 2H).

### Example 97: 2-Methyl-5-((1-methylpyrrolidin-2-yl)methoxy)-N-(1-(naphthalen-1-yl)cyclopropyl)benzamide (Compound 283)

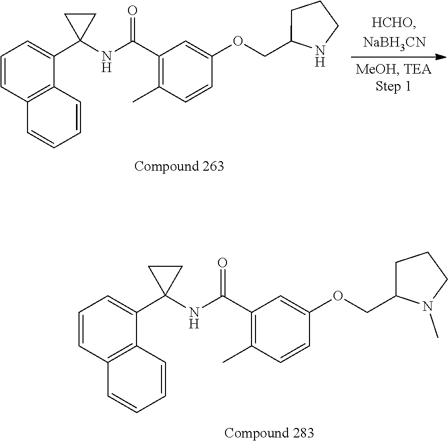 [SMILES: Cc1ccc(OCC2CCCN2)cc1C(=O)NC1(c2cccc3ccccc23)CC1 ; Cc1ccc(OCC2CCCN2C)cc1C(=O)NC1(c2cccc3ccccc23)CC1]

### Step 1: 2-Methyl-5-((1-methylpyrrolidin-2-yl)methoxy)-N-(1-(naphthalen-1-yl)cyclopropyl)benzamide (Compound 283)

To a solution of 2-methyl-N-(1-(naphthalen-1-yl)cyclopropyl)-5-(pyrrolidin-2-ylmethoxy)benzamide (160 mg, 200 μmol, 1.0 eq) in MeOH (4 mL) was added TEA (200 μL), followed by the addition of formaldehyde (32.4 mg, 400 μmol, 29.8 μL, 37% purity in water, 2.0 eq). The resulting mixture was adjusted to pH 6 with a small amount of AcOH. The mixture was stirred at 20° C. for 30 min, then NaBH₃CN (25.1 mg, 400 μmol, 2.0 eq) was added. The reaction mixture was stirred at 20° C. for another 5 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by preparative HPLC (Phenomenex C18 column (75×30 mm, 3 μm); flow rate: 25 mL/min; gradient: 25%-55% B over 8 min; mobile phase A: 10 mM aqueous NH₄HCO₃, mobile phase B: acetonitrile) to give 2-methyl-5-((1-methylpyrrolidin-2-yl)methoxy)-N-(1-(naphthalen-1-yl)cyclopropyl)benzamide (35.9 mg, 86.6 μmol, 43% yield) as a yellow gum. M+H⁺=415.1 (LCMS); ¹H NMR (400 MHZ, DMSO-d₆) δ 9.07 (s, 1H), 8.65 (s, 1H), 7.96-7.90 (m, 1H), 7.85-7.79 (m, 2H), 7.59-7.42 (m, 3H), 7.04-6.99 (m, 1H), 6.82 (dd, J=2.8, 8.4 Hz, 1H), 6.60 (d, J=2.6 Hz, 1H), 3.91-3.68 (m, 2H), 2.92 (td, J=4.3, 9.1 Hz, 1H), 2.53-2.52 (m, 1H), 2.30 (s, 3H), 2.15 (d, J=8.5 Hz, 1H), 2.07-1.73 (m, 4H), 1.72-1.59 (m, 2H), 1.57-1.45 (m, 1H), 1.40-1.32 (m, 2H), 1.22-1.10 (m, 2H).

### Example 98: 2-Methyl-N-(1-(naphthalen-1-yl)cyclopropyl)-5-(piperidin-3-yloxy)benzamide (Compound 268)

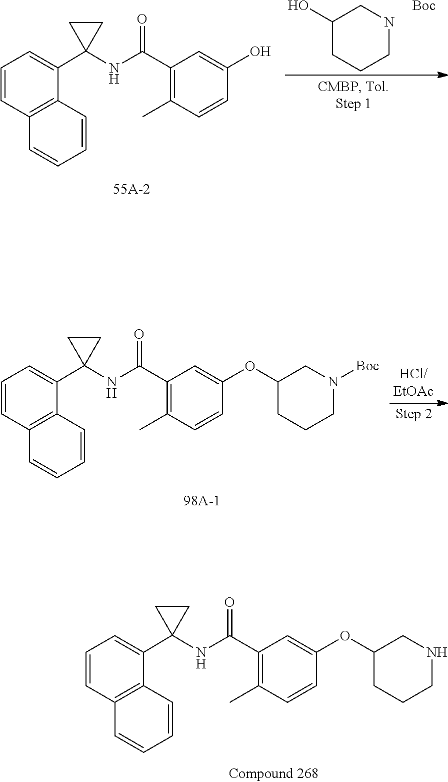 [SMILES: Cc1ccc(O)cc1C(=O)NC1(c2cccc3ccccc23)CC1 ; OC1CCCNC1 ; Cc1ccc(OC2CCCN(C(=O)OC(C)(C)C)C2)cc1C(=O)NC1(c2cccc3ccccc23)CC1 ; Cc1ccc(OC2CCCNC2)cc1C(=O)NC1(c2cccc3ccccc23)CC1 ; "Boc"]

### Step 1: tert-Butyl 3-(4-methyl-3-((1-(naphthalen-1-yl)cyclopropyl)carbamoyl)phenoxy) piperidine-1-carboxylate (98A-1)

To a solution of 5-hydroxy-2-methyl-N-(1-(naphthalen-1-yl)cyclopropyl)benzamide (200 mg, 630 μmol, 1.0 eq) and tert-butyl 3-hydroxypiperidine-1-carboxylate (190 mg, 945 μmol, 1.5 eq) in toluene (3.0 mL) was added CMBP (228 mg, 945 μmol, 1.5 eq) at 25° C. The mixture was stirred at 110° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×3). The combined organic layers were washed with brine (10 mL×3), dried over Na₂SO₄, filtered, and concentrated under vacuum to give the crude product tert-butyl 3-(4-methyl-3-((1-(naphthalen-1-yl)cyclopropyl)carbamoyl)phenoxy) piperidine-1-carboxylate (125 mg), which was used in the next step without any further purification. M+H⁺=445.2 (LCMS).

### Step 2: 2-Methyl-N-(1-(naphthalen-1-yl)cyclopropyl)-5-(piperidin-3-yloxy)benzamide (Compound 268)

To a mixture of tert-butyl 3-(4-methyl-3-((1-(naphthalen-1-yl)cyclopropyl)carbamoyl) phenoxy) piperidine-1-carboxylate (125 mg, 250 μmol, 1.0 eq) in EtOAc (2.0 mL) was added HCl/EtOAc (4 M, 4.0 mL). The mixture was stirred at 25° C. for 1 h. LCMS indicated that the starting material was completely consumed, and the desired mass was detected. The reaction mixture was concentrated under vacuum to give a residue which was purified by preparative HPLC (Phenomenex Luna column (80×30 mm, 3 μm); flow rate: 25 mL/min; gradient: 20%-45% B over 8 min; mobile phase A: 0.04% aqueous HCl, mobile phase B: acetonitrile). 2-Methyl-N-(1-(naphthalen-1-yl)cyclopropyl)-5-(piperidin-3-yloxy)benzamide (42.4 mg, 106 μmol, 42% yield, HCl salt) was obtained as a yellow solid. M+H⁺=401.2 (LCMS); ¹H NMR (400 MHZ, DMSO-d) δ 9.32-9.20 (m, 1H), 9.14 (s, 1H), 8.65 (br d, J=8.3 Hz, 2H), 7.93 (d, J=7.8 Hz, 1H), 7.86-7.79 (m, 2H), 7.61-7.49 (m, 2H), 7.46 (t, J=7.6 Hz, 1H), 7.07 (d, J=8.4 Hz, 1H), 6.97-6.89 (m, 1H), 6.71 (d, J=2.6 Hz, 1H), 4.62 (br s, 1H), 3.28-3.16 (m, 1H), 3.15-3.04 (m, 1H), 3.00 (br s, 2H), 1.95 (s, 3H), 1.87-1.77 (m, 2H), 1.77-1.69 (m, 1H), 1.62 (br t, J=10.4 Hz, 1H), 1.36 (s, 2H), 1.23-1.12 (m, 2H).

### Example 99: 2-Methyl-5-((1-methylpiperidin-3-yl)oxy)-N-(1-(naphthalen-1-yl)cyclopropyl)benzamide (Compound 289)

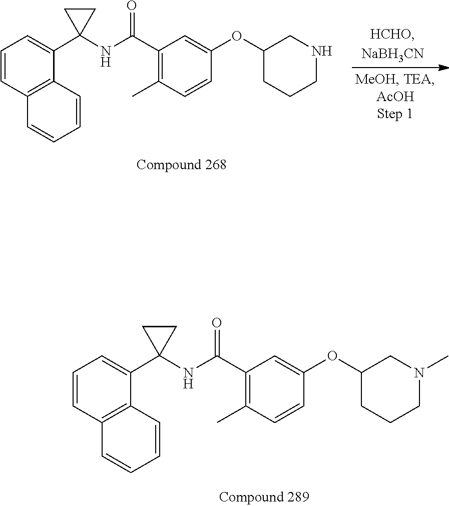 [SMILES: Cc1ccc(OC2CCCNC2)cc1C(=O)NC1(c2cccc3ccccc23)CC1 ; Cc1ccc(OC2CCCN(C)C2)cc1C(=O)NC1(c2cccc3ccccc23)CC1]

### Step 1: 2-Methyl-5-((1-methylpiperidin-3-yl)oxy)-N-(1-(naphthalen-1-yl)cyclopropyl)benzamide (Compound 289)

To a solution of 2-methyl-N-(1-(naphthalen-1-yl)cyclopropyl)-5-(piperidin-3-yloxy)benzamide (15.0 mg, 37.5 μmol, 1.0 eq) in MeOH (1.0 mL) was added TEA (300 μL), followed by the addition of formaldehyde (2.25 mg, 74.9 μmol, 2.06 μL, 37% purity in water, 2.0 eq). The resulting mixture was adjusted to pH 6 with a small amount of AcOH. The mixture was stirred at 20° C. for 30 min, then NaBH₃CN (4.71 mg, 74.9 μmol, 2.0 eq) was added. The reaction mixture was stirred at 20° C. for another 16 h. LCMS indicated that the starting material was completely consumed, and the desired mass was detected. The reaction mixture was poured into water (3.0 mL) and extracted with EtOAc (3.0 mL×3). The combined organic layers were washed with brine (3.0 mL×3), dried over Na₂SO₄, and concentrated under vacuum to give a residue which was purified by preparative HPLC (Waters Xbridge BEH C18 column (100×30 mm, 10 μm); flow rate: 25 mL/min; gradient: 35%-65% B over 8 min; mobile phase A: 10 mM aqueous NH₄HCO₃, mobile phase B: acetonitrile). 2-Methyl-5-((1-methylpiperidin-3-yl)oxy)-N-(1-(naphthalen-1-yl)cyclopropyl)benzamide (4.70 mg, 11.3 μmol, 30% yield) was obtained as a white solid. M+H⁺=415.2 (LCMS); ¹H NMR (400 MHZ, DMSO-d₆) δ 9.07 (s, 1H), 8.65 (d, J=8.3 Hz, 1H), 7.97-7.74 (m, 3H), 7.61-7.43 (m, 3H), 7.01 (d, J=8.4 Hz, 1H), 6.86-6.81 (m, 1H), 6.59 (d, J=2.5 Hz, 1H), 4.27-4.20 (m, 1H), 2.75 (br d, J=8.4 Hz, 1H), 2.14 (s, 3H), 2.03-1.60 (m, 8H), 1.56-1.42 (m, 1H), 1.35 (br s, 2H), 1.25 (br d, J=11.0 Hz, 1H), 1.17 (br s, 2H).

### Example 100: 2-Methyl-N-(1-(naphthalen-1-yl)cyclopropyl)-5-(piperidin-2-ylmethoxy)benzamide (Compound 339)

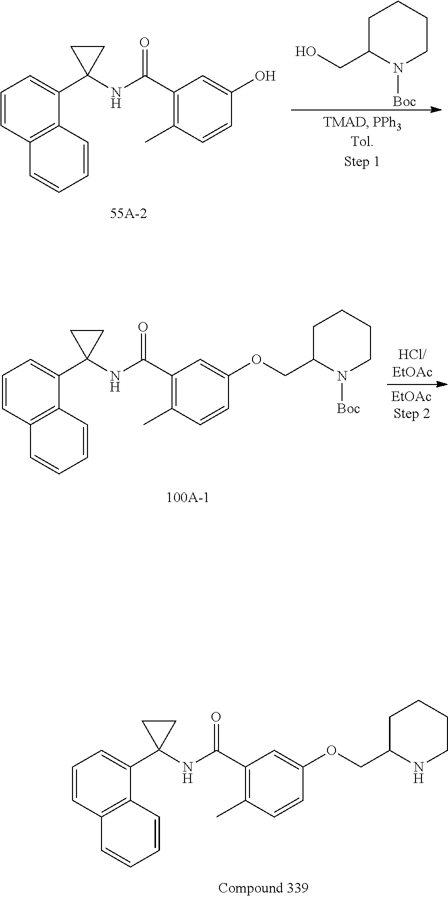 [SMILES: Cc1ccc(O)cc1C(=O)NC1(c2cccc3ccccc23)CC1 ; CC(C)(C)OC(=O)N1CCCCC1CO ; Cc1ccc(OCC2CCCCN2C(=O)OC(C)(C)C)cc1C(=O)NC1(c2cccc3ccccc23)CC1 ; Cc1ccc(OCC2CCCCN2)cc1C(=O)NC1(c2cccc3ccccc23)CC1]

### Step 1: tert-Butyl 2-((4-methyl-3-((1-(naphthalen-1-yl)cyclopropyl)carbamoyl)phenoxy)methyl)piperidine-1-carboxylate (100A-1)

To a solution of 5-hydroxy-2-methyl-N-(1-(naphthalen-1-yl)cyclopropyl)benzamide (450 mg, 1.42 mmol, 1.0 eq) and tert-butyl 2-(hydroxymethyl) piperidine-1-carboxylate (915 mg, 4.26 mmol, 3.0 eq) in toluene (15 mL) were added TMAD (732 mg, 4.26 mmol, 3.0 eq) and PPh₃ (1.12 g, 4.26 mmol, 3.0 eq). The mixture was degassed and purged with N₂ three times and stirred at 100° C. for 16 h under a N₂ atmosphere. LCMS indicated that the starting material was completely consumed, and the desired mass was detected. The reaction mixture was allowed to cool to room temperature, poured into H₂O (12 mL) and extracted with EtOAc (9.0 mL×3). The combined organic layers were dried over Na₂SO₄, filtered, and concentrated under vacuum to give a residue which was purified by flash silica gel chromatography using a gradient of EtOAc/petroleum ether from 1/100 to 4/5. tert-Butyl 2-((4-methyl-3-((1-(naphthalen-1-yl)cyclopropyl)carbamoyl)phenoxy)methyl) piperidine-1-carboxylate (113 mg, 15% yield) was obtained as a white solid. M+H⁺=515.3 (LCMS).

### Step 2: 2-Methyl-N-(1-(naphthalen-1-yl)cyclopropyl)-5-(piperidin-2-ylmethoxy)benzamide (Compound 339)

To a solution of tert-butyl 2-((4-methyl-3-((1-(naphthalen-1-yl)cyclopropyl)carbamoyl) phenoxy)methyl) piperidine-1-carboxylate (113 mg, 220 μmol, 1.0 eq) in EtOAc (2.0 mL) was added HCl/EtOAc (4 M, 5.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna (80×30 mm, 3 μm); flow rate: 25 mL/min; gradient: 10%-60% B over 8 min; mobile phase A: 0.04% aqueous HCl, mobile phase B: acetonitrile). 2-Methyl-N-(1-(naphthalen-1-yl)cyclopropyl)-5-(piperidin-2-ylmethoxy)benzamide (15.7 mg, 34.8 μmol, 16% yield, HCl salt) was obtained as a white solid. M+H⁺=415.1 (LCMS); 1H NMR (400 MHZ, DMSO-d₆) δ 9.15-9.09 (m, 1H), 8.65 (d, J=8.6 Hz, 1H), 7.98-7.91 (m, 1H), 7.86-7.80 (m, 2H), 7.60-7.44 (m, 3H), 7.11-7.06 (m, 1H), 6.93-6.87 (m, 1H), 6.69-6.65 (m, 1H), 4.13-4.02 (m, 1H), 3.98-3.87 (m, 1H), 3.49-3.41 (m, 1H), 3.23 (br d, J=14.1 Hz, 1H), 2.98-2.83 (m, 1H), 1.97 (s, 3H), 1.87-1.68 (m, 3H), 1.59-1.44 (m, 3H), 1.39-1.33 (m, 2H), 1.20-1.17 (m, 2H).

### Example 101: 2-Methyl-5-((1-methylpiperidin-2-yl)methoxy)-N-(1-(naphthalen-1-yl)cyclopropyl)benzamide (Compound 288) and 2-methyl-5-((1-methylazepan-3-yl)oxy)-N-(1-(naphthalen-1-yl)cyclopropyl)benzamide (Compound 293)

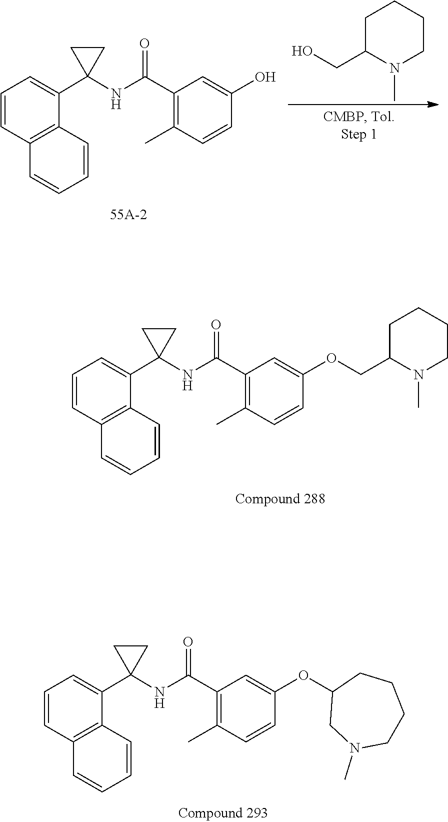

### Step 1: 2-Methyl-5-((1-methylpiperidin-2-yl)methoxy)-N-(1-(naphthalen-1-yl)cyclopropyl)benzamide (Compound 288) and 2-methyl-5-((1-methylazepan-3-yl)oxy)-N-(1-(naphthalen-1-yl)cyclopropyl)benzamide (Compound 293)

To a solution of 5-hydroxy-2-methyl-N-(1-(naphthalen-1-yl)cyclopropyl)benzamide (70.0 mg, 221 μmol, 1.0 eq) and (1-methylpiperidin-2-yl) methanol (28.5 mg, 221 μmol, 29.0 μL, 1.0 eq) in toluene (3.5 mL) was added CMBP (80.0 mg, 331 μmol, 1.5 eq). The mixture was degassed and purged with N₂ three times and stirred at 110° C. for 16 h under a N₂ atmosphere. LCMS indicated that the starting material was completely consumed, and the desired mass was detected. The reaction mixture was allowed to cool to room temperature, poured into H₂O (4.0 mL) and extracted with EtOAc (3.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 30%-60% B over 8 min; mobile phase A: 10 mM aqueous NH₄HCO₃, mobile phase B: acetonitrile). 2-Methyl-5-((1-methylpiperidin-2-yl)methoxy)-N-(1-(naphthalen-1-yl)cyclopropyl)benzamide (12.3 mg, 28.7 μmol, 13% yield) was obtained as a yellow solid. M+H⁺=429.3 (LCMS); ¹H NMR (400 MHZ, DMSO-d₆) δ 9.09 (s, 1H), 8.68-8.63 (m, 1H), 7.96-7.90 (m, 1H), 7.87-7.78 (m, 2H), 7.62-7.42 (m, 3H), 7.02 (d, J=8.4 Hz, 1H), 6.83 (dd, J=2.7, 8.3 Hz, 1H), 6.62-6.57 (m, 1H), 3.93 (br d, J=4.5 Hz, 1H), 3.78 (br d, J=4.9 Hz, 1H), 2.76-2.68 (m, 1H), 2.19-2.08 (m, 4H), 1.95 (s, 4H), 1.66 (br d, J=10.0 Hz, 2H), 1.57-1.47 (m, 1H), 1.45-1.10 (m, 7H). 2-Methyl-5-((1-methylazepan-3-yl)oxy)-N-(1-(naphthalen-1-yl)cyclopropyl)benzamide (17.6 mg, 41.1 μmol, 19% yield) was obtained as a white solid. M+H⁺=429.3 (LCMS); ¹H NMR (400 MHZ, DMSO-d₆) δ 9.09 (s, 1H), 8.74-8.58 (m, 1H), 7.93 (d, J=7.8 Hz, 1H), 7.81 (dd, J=7.8, 11.7 Hz, 2H), 7.61-7.41 (m, 3H), 7.11-6.95 (m, 1H), 6.79 (dd, J=2.8, 8.4 Hz, 1H), 6.54 (d, J=2.6 Hz, 1H), 4.49-4.27 (m, 1H), 2.79-2.63 (m, 2H), 2.61-2.56 (m, 1H), 2.47-2.35 (m, 1H), 2.26-2.17 (m, 3H), 2.02-1.93 (m, 3H), 1.92-1.83 (m, 1H), 1.69-1.52 (m, 4H), 1.48-1.40 (m, 1H), 1.38-1.30 (m, 2H), 1.20-1.09 (m, 2H).

### Example 102: 2-Methyl-5-(morpholin-3-ylmethoxy)-N-(1-(naphthalen-1-yl)cyclopropyl)benzamide (Compound 286)

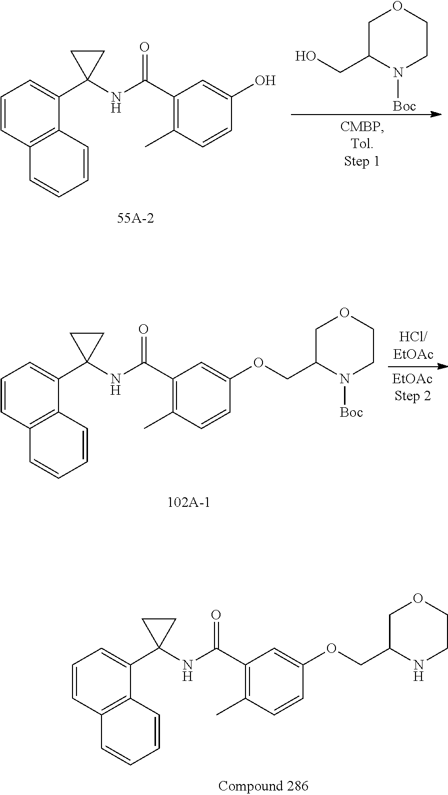 [SMILES: Cc1ccc(O)cc1C(=O)NC1(c2cccc3ccccc23)CC1 ; CC(C)(C)OC(=O)N1CCOCC1CO ; Cc1ccc(OCC2COCCN2C(=O)OC(C)(C)C)cc1C(=O)NC1(c2cccc3ccccc23)CC1 ; Cc1ccc(OCC2COCCN2)cc1C(=O)NC1(c2cccc3ccccc23)CC1]

### Step 1: tert-Butyl 3-((4-methyl-3-((1-(naphthalen-1-yl)cyclopropyl)carbamoyl)phenoxy)methyl)morpholine-4-carboxylate (102A-1)

To a solution of 5-hydroxy-2-methyl-N-(1-(naphthalen-1-yl)cyclopropyl)benzamide (100 mg, 315 μmol, 1.0 eq) and tert-butyl 3-(hydroxymethyl) morpholine-4-carboxylate (68.5 mg, 315 μmol, 1.0 eq) in toluene (5.0 mL) was added CMBP (114 mg, 473 μmol, 1.5 eq). The mixture was degassed and purged with N₂ three times and stirred at 110° C. for 16 h under a N₂ atmosphere. LCMS indicated that the starting material was completely consumed, and the desired mass was detected. The reaction mixture was allowed to cool to room temperature, poured into H₂O (5.0 mL) and extracted with EtOAc (2.0 mL×3). The combined organic layers were dried over Na₂SO₄, filtered, and concentrated under vacuum to give a residue which was purified by preparative TLC (EtOAc/petroleum ether=1/1, R_{f}=0.6). tert-Butyl 3-((4-methyl-3-((1-(naphthalen-1-yl)cyclopropyl)carbamoyl)phenoxy)methyl) morpholine-4-carboxylate (40.0 mg, 77.4 μmol, 25% yield) was obtained as a yellow oil. M+H⁺=517.2 (LCMS).

### Step 2: 2-Methyl-5-(morpholin-3-ylmethoxy)-N-(1-(naphthalen-1-yl)cyclopropyl)benzamide (Compound 286)

To a solution of tert-butyl 3-((4-methyl-3-((1-(naphthalen-1-yl)cyclopropyl)carbamoyl) phenoxy)methyl) morpholine-4-carboxylate (40.0 mg, 77.4 μmol, 1.0 eq) in EtOAc (2.0 mL) was added HCl/EtOAc (4 M, 4.0 mL). The mixture was stirred at 20° C. for 2 h. LCMS indicated that the starting material was completely consumed, and the desired mass was detected. The mixture was concentrated under vacuum at 30° C. to give a residue which was purified by preparative HPLC (Phenomenex Luna (80×30 mm, 3 μm); flow rate: 25 mL/min; gradient: 20%-60% B over 8 min; mobile phase A: 0.04% aqueous HCl, mobile phase B: acetonitrile). 2-Methyl-5-(morpholin-3-ylmethoxy)-N-(1-(naphthalen-1-yl)cyclopropyl)benzamide (22.9 mg, 49.9 μmol, 65% yield, HCl salt) was obtained as a white solid. M+H⁺=417.1 (LCMS); ¹H NMR (400 MHZ, DMSO-d₆) δ 9.56-9.41 (m, 1H), 9.40-9.24 (m, 1H), 9.13 (s, 1H), 8.65 (d, J=8.3 Hz, 1H), 7.93 (d, J=7.8 Hz, 1H), 7.83 (dd, J=4.1, 7.6 Hz, 2H), 7.64-7.42 (m, 3H), 7.08 (d, J=8.4 Hz, 1H), 6.90 (dd, J=2.6, 8.4 Hz, 1H), 6.68 (d, J=2.6 Hz, 1H), 4.18-3.87 (m, 4H), 3.75-3.56 (m, 3H), 3.26-3.07 (m, 2H), 1.96 (s, 3H), 1.40-1.34 (m, 2H), 1.21-1.15 (m, 2H).

### Example 103: 2-Methyl-5-((4-methylmorpholin-3-yl)methoxy)-N-(1-(naphthalen-1-yl)cyclopropyl)benzamide (Compound 302)

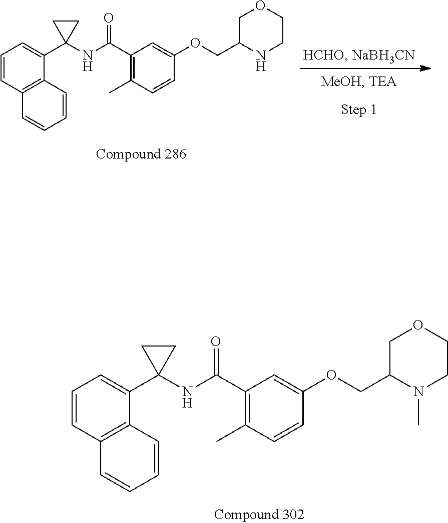 [SMILES: Cc1ccc(OCC2COCCN2)cc1C(=O)NC1(c2cccc3ccccc23)CC1 ; Cc1ccc(OCC2COCCN2C)cc1C(=O)NC1(c2cccc3ccccc23)CC1]

### Step 1: 2-Methyl-5-((4-methylmorpholin-3-yl)methoxy)-N-(1-(naphthalen-1-yl)cyclopropyl)benzamide (Compound 302)

To a solution of 2-methyl-5-(morpholin-3-ylmethoxy)-N-(1-(naphthalen-1-yl)cyclopropyl)benzamide (40.0 mg, 96.0 μmol, 1.0 eq) in MeOH (3.0 mL) was added TEA (10.0 μL), followed by the addition of formaldehyde (15.6 mg, 192 μmol, 14.3 μL, 37% purity in water, 2.0 eq). The resulting mixture was adjusted to pH 6 with a small amount of AcOH. The mixture was stirred at 20° C. for 30 min, then NaBH₃CN (12.1 mg, 192 μmol, 2.0 eq) was added. The reaction mixture was stirred at 20° C. for another 1 h. LCMS indicated that the starting material was completely consumed, and the desired mass was detected. The reaction mixture was poured into H₂O (3.0 mL) and extracted with EtOAc (1.0 mL×3). The combined organic layers were dried over Na₂SO₄, filtered, and concentrated under vacuum to give a residue which was purified by preparative HPLC (Phenomenex Luna C18 (75×30 mm, 3 μm); flow rate: 25 mL/min; gradient: 1%-70% B over 8 min; mobile phase A: 0.2% aqueous FA, mobile phase B: acetonitrile). 2-Methyl-5-((4-methylmorpholin-3-yl)methoxy)-N-(1-(naphthalen-1-yl)cyclo propyl)benzamide (22.9 mg, 52.1 μmol, 54% yield, FA salt) was obtained as a white solid. M+H⁺=431.1 (LCMS); ¹H NMR (400 MHZ, DMSO-d₆) δ 9.09 (s, 1H), 8.65 (d, J=8.3 Hz, 1H), 7.93 (d, J=7.3 Hz, 1H), 7.82 (t, J=8.4 Hz, 2H), 7.61-7.43 (m, 3H), 7.03 (d, J=8.4 Hz, 1H), 6.84 (dd, J=2.8, 8.4 Hz, 1H), 6.61 (d, J=2.7 Hz, 1H), 4.00 (dd, J=4.2, 10.3 Hz, 1H), 3.81-3.73 (m, 2H), 3.71-3.63 (m, 1H), 3.48 (dt, J=2.4, 10.7 Hz, 1H), 3.28 (dd, J=9.5, 10.9 Hz, 1H), 2.68-2.59 (m, 1H), 2.37-2.30 (m, 1H), 2.26-2.13 (m, 4H), 1.95 (s, 3H), 1.35 (s, 2H), 1.21-1.12 (m, 2H).

### Example 105: 5-((Hexahydro-1H-pyrrolizin-7a-yl)methoxy)-2-methyl-N-(1-(naphthalen-1-yl)cyclopropyl)benzamide (Compound 376)

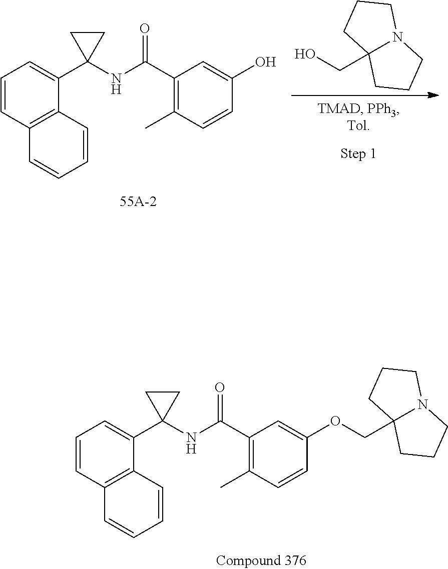 [SMILES: Cc1ccc(O)cc1C(=O)NC1(c2cccc3ccccc23)CC1 ; OCC12CCCN1CCC2 ; Cc1ccc(OCC23CCCN2CCC3)cc1C(=O)NC1(c2cccc3ccccc23)CC1]

### Step 1: 5-((Hexahydro-1H-pyrrolizin-7a-yl)methoxy)-2-methyl-N-(1-(naphthalen-1-yl)cyclopropyl)benzamide (Compound 376)

A mixture of 5-hydroxy-2-methyl-N-(1-(naphthalen-1-yl)cyclopropyl)benzamide (70.0 mg, 221 μmol, 1.0 eq), (hexahydro-1H-pyrrolizin-7a-yl) methanol (62.3 mg, 441 μmol, 2.0 eq) TMAD (114 mg, 662 μmol, 3.0 eq) and PPh₃ (174 mg, 662 μmol, 3.0 eq) in toluene (5.0 mL) was degassed and purged with N₂ three times. The mixture was stirred at 100° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×5). The combined organic layers were dried over Na₂SO₄, filtered, and concentrated under vacuum to give a residue which was purified by preparative HPLC (Phenomenex C18 column (75×30 mm, 3 μm); flow rate: 25 mL/min; gradient: 15%-50% B over 8 min; mobile phase A: 10 mM aqueous NH₄HCO₃, mobile phase B: acetonitrile) to give 5-((hexahydro-1H-pyrrolizin-7a-yl)methoxy)-2-methyl-N-(1-(naphthalen-1-yl)cyclo propyl)benzamide (17.1 mg, 38.6 μmol, 18% yield) as a yellow solid. M+H⁺=441.3 (LCMS); ¹H NMR (400 MHZ, DMSO-d₆) δ 9.06 (s, 1H), 8.67 (d, J=8.3 Hz, 1H), 7.93 (d, J=7.9 Hz, 1H), 7.88-7.75 (m, 2H), 7.61-7.40 (m, 3H), 6.98-6.95 (m, 1H), 7.00 (d, J=8.4 Hz, 1H), 6.81 (dd, J=2.7, 8.3 Hz, 1H), 6.60 (d, J=2.6 Hz, 1H), 3.51 (s, 2H), 2.95-2.83 (m, 2H), 2.56-2.51 (m, 2H), 1.95 (s, 3H), 1.87-1.63 (m, 6H), 1.60-1.46 (m, 2H), 1.41-1.31 (m, 2H), 1.23-1.11 (m, 2H).

### Example 106: 5-(2-(Dimethylamino)ethoxy)-2-methoxy-N-(1-(naphthalen-1-yl)cyclopropyl)benzamide (Compound 303)

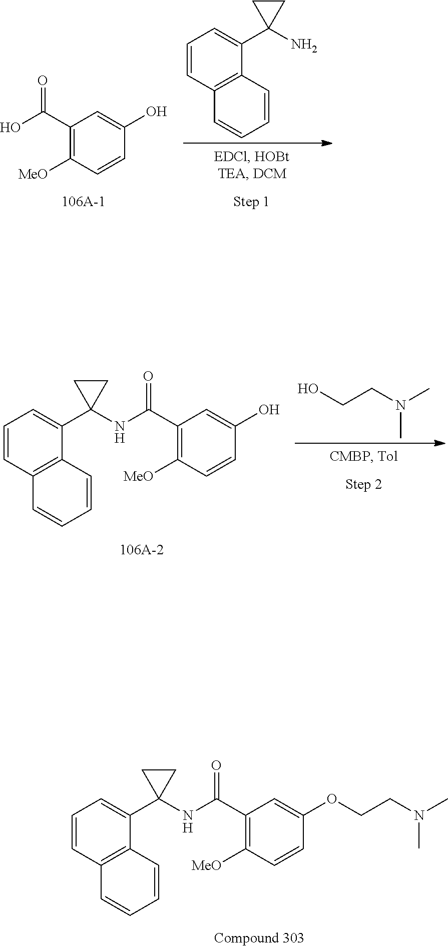

### Step 1: 5-Hydroxy-2-methoxy-N-(1-(naphthalen-1-yl)cyclopropyl)benzamide (106A-2)

To a solution of 5-hydroxy-2-methoxybenzoic acid (100 mg, 595 μmol, 1.0 eq) and 1-(naphthalen-1-yl)cyclopropanamine (109 mg, 595 μmol, 1.0 eq) in DMF (3.0 mL) was added TEA (60.2 mg, 595 μmol, 82.8 μL, 1.0 eq), followed by EDCI (120 mg, 625 μmol, 1.1 eq) and HOBt (16.1 mg, 119 μmol, 0.2 eq). The mixture was stirred at 20° C. for 16 h. LCMS indicated that the starting material was completely consumed, and the desired mass was detected. The reaction mixture was poured into H₂O (5.0 mL) and extracted with EtOAc (10 mL×3). The combined organic layers were dried over Na₂SO₄, filtered, and concentrated under vacuum to give a residue which was purified by preparative TLC (EtOAc/petroleum ether=1/2, R_{f}=0.5). 5-Hydroxy-2-methoxy-N-(1-(naphthalen-1-yl)cyclopropyl)benzamide (120 mg) was obtained as a yellow oil. M+H⁺=334.1 (LCMS).

### Step 2: 5-(2-(Dimethylamino)ethoxy)-2-methoxy-N-(1-(naphthalen-1-yl)cyclopropyl)benzamide (Compound 303)

To a solution of 5-hydroxy-2-methoxy-N-(1-(naphthalen-1-yl)cyclopropyl)benzamide (120 mg, 360 μmol, 1.0 eq) and 2-(dimethylamino) ethanol (35.3 mg, 396 μmol, 39.7 μL, 1.1 eq) in toluene (4.0 mL) was added CMBP (95.6 mg, 396 μmol, 1.1 eq). The mixture was degassed and purged with N₂ three times, and then was stirred at 110° C. for 16 h under a N₂ atmosphere. LCMS indicated that the starting material was completely consumed, and the desired mass was detected. The reaction mixture was allowed to cool to room temperature, poured into H₂O (15 mL) and extracted with EtOAc (10 mL×5). The combined organic layers were dried over Na₂SO₄, filtered, and concentrated under vacuum to give a residue which was purified by preparative HPLC (Phenomenex Gemini C18 column (80×40 mm, 3 μm); flow rate: 25 mL/min; gradient: 15%-45% B over 8 min; mobile phase A: 10 mM aqueous NH₄HCO₃, mobile phase B: acetonitrile). 5-(2-(Dimethylamino)ethoxy)-2-methoxy-N-(1-(naphthalen-1-yl)cyclopropyl)benzamide (81.0 mg, 181 μmol, 50% yield) was obtained as a brown solid. M+H⁺=405.1 (LCMS); ¹H NMR (400 MHZ, DMSO-d₆) δ 8.98-8.92 (m, 1H), 8.63-8.55 (m, 1H), 7.97-7.90 (m, 1H), 7.86-7.76 (m, 2H), 7.67-7.59 (m, 1H), 7.57-7.38 (m, 2H), 7.09-7.03 (m, 1H), 6.99-6.91 (m, 2H), 3.97-3.89 (m, 2H), 3.74-3.65 (m, 3H), 2.56-2.54 (m, 2H), 2.24-2.10 (m, 6H), 1.42-1.35 (m, 2H), 1.24-1.15 (m, 2H).

### Example 107: 2-Bromo-5-(2-(dimethylamino)ethoxy)-N-(1-(naphthalen-1-yl)cyclopropyl)benzamide (Compound 269)

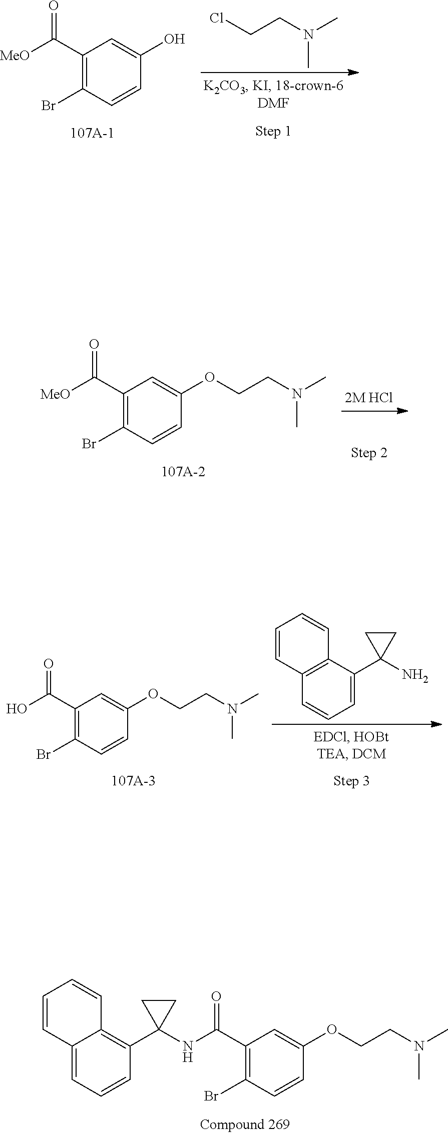

### Step 1: Methyl 2-bromo-5-(2-(dimethylamino)ethoxy)benzoate (107A-2)

To a solution of 2-chloro-N,N-dimethylethanamine (1.25 g, 8.66 mmol, 1.0 eq, HCl salt) in DMF (10 mL) was added K₂CO₃ (5.98 g, 43.3 mmol, 5.0 eq). The mixture was stirred at 25° C. for 30 min, then 18-crown-6 (3.62 g, 13.7 mmol, 1.6 eq), KI (2.41 g, 14.5 mmol, 1.7 eq) and methyl 2-bromo-5-hydroxybenzoate (2.00 g, 8.66 mmol, 1.0 eq) were added. The mixture was stirred at 70° C. for 5 h. TLC indicated that that the starting material was completely consumed. The reaction mixture was allowed to cool to room temperature, poured into H₂O (10 mL) at 25° C. and extracted with EtOAc (10 mL×3). The combined organic layers were washed with brine (10 mL×3), dried over Na₂SO₄, filtered, and concentrated under vacuum to give a residue which was purified by flash silica gel chromatography using a gradient of EtOAc/petroleum ether from 0/1 to 1/0. Methyl 2-bromo-5-(2-(dimethylamino)ethoxy)benzoate (570 mg, 1.32 mmol, 22% yield) was obtained as a white solid. M+H⁺=302.0 (LCMS).

### Step 2: 2-Bromo-5-(2-(dimethylamino)ethoxy)benzoic acid (107A-3)

A mixture of methyl 2-bromo-5-(2-(dimethylamino)ethoxy)benzoate (570 mg, 1.89 mmol, 1.0 eq) in HCl (2 M aqueous, 15 mL) was stirred at 100° C. for 3 h. LCMS indicated that the starting material was completely consumed, and the desired mass was detected. The reaction mixture was allowed to cool to room temperature and treated with NaOH (2 M aqueous) to adjust the pH to 6. The resulting mixture was concentrated under vacuum to remove the water completely. The resulting mixture was treated with DCM/MeOH (V/V=10/1, 15 mL) then filtered. The filter cake was washed with DCM/MeOH (15 mL×2) to ensure that all product was washed from the solids. The combined organic layers were concentrated under vacuum to give the crude product 2-bromo-5-(2-(dimethylamino)ethoxy)benzoic acid (500 mg), which was used in the next step without any further purification. M+H⁺=288.1 (LCMS).

### Step 3: 2-Bromo-5-(2-(dimethylamino)ethoxy)-N-(1-(naphthalen-1-yl)cyclopropyl)benzamide (Compound 269)

To a solution of 2-bromo-5-(2-(dimethylamino)ethoxy)benzoic acid (100 mg, 292 μmol, 1.2 eq) in DCM (1.0 mL) were added 1-(naphthalen-1-yl)cyclopropanamine (44.5 mg, 243 μmol, 1.0 eq), EDCI (69.9 mg, 364 μmol, 1.5 eq), HOBt (49.2 mg, 364 μmol, 1.5 eq) and TEA (73.8 mg, 729 μmol, 101 μL, 3.0 eq). The mixture was stirred at 25° C. for 16 h. LCMS indicated that the starting material was completely consumed, and the desired mass was detected. The reaction mixture was poured into H₂O (10 mL) at 25° C. and extracted with EtOAc (10 mL×3). The combined organic layers were washed with brine (10 mL×3), dried over Na₂SO₄, filtered, and concentrated under vacuum to give a residue which was purified by preparative HPLC (Phenomenex Luna column (80×30 mm, 3 μm); flow rate: 25 mL/min; gradient: 15%-35% B over 8 min; mobile phase A: 0.04% aqueous HCl, mobile phase B: acetonitrile). 2-Bromo-5-(2-(dimethylamino)ethoxy)-N-(1-(naphthalen-1-yl)cyclopropyl)benzamide (17.2 mg, 37.9 μmol, 16% yield, HCl salt) was obtained as a white solid. M+H⁺=453.1 (LCMS); ¹H NMR (400 MHZ, DMSO-d₆) δ 10.45-10.29 (m, 1H), 9.31 (s, 1H), 8.62 (d, J=8.2 Hz, 1H), 7.93 (d, J=7.8 Hz, 1H), 7.86-7.79 (m, 2H), 7.58-7.44 (m, 4H), 6.98-6.93 (m, 1H), 6.74 (d, J=3.1 Hz, 1H), 4.28 (t, J=4.8 Hz, 2H), 3.45-3.39 (m, 2H), 2.76 (d, J=4.8 Hz, 6H), 1.40 (s, 2H), 1.21-1.14 (m, 2H).

### Example 108: 5-(2-(Dimethylamino)ethoxy)-2-ethynyl-N-(1-(naphthalen-1-yl)cyclopropyl)benzamide (Compound 337)

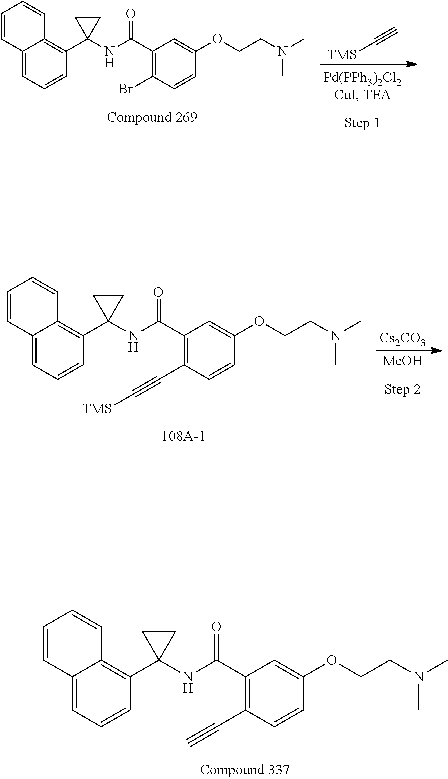

### Step 1: 5-(2-(Dimethylamino)ethoxy)-N-(1-(naphthalen-1-yl)cyclopropyl)-2-((trimethylsilyl)ethynyl)benzamide (108A-1)

To a solution of 2-bromo-5-(2-(dimethylamino)ethoxy)-N-(1-(naphthalen-1-yl)cyclopropyl)benzamide (200 mg, 408 μmol, 1.0 eq, HCl salt) in a mixture of toluene (16 mL) and H₂O (4.0 mL) were added ethynyltrimethylsilane (80.2 mg, 816 μmol, 113 μL, 2.0 eq), Pd(PPh₃)₂Cl₂ (28.7 mg, 40.8 μmol, 0.1 eq), CuI (7.78 mg, 40.8 μmol, 0.1 eq) and TEA (207 mg, 2.04 mmol, 284 μL, 5.0 eq) under a N₂ atmosphere. The resulting mixture was stirred at 80° C. for 16 h. LCMS indicated that 48% of the starting material remained and 32% of desired product was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by preparative HPLC (Waters Xbridge Prep OBD C18 column (150×40 mm, 10 μm); flow rate: 25 mL/min; gradient: 70%-98% B over 8 min; mobile phase A: 10 mM aqueous NH₄HCO₃, mobile phase B: acetonitrile). 5-(2-(Dimethylamino)ethoxy)-N-(1-(naphthalen-1-yl)cyclopropyl)-2-((trimethylsilyl)ethynyl)benzamide (50.0 mg, 106 μmol, 26% yield) was obtained as a white solid. M+H⁺=471.2 (LCMS).

### Step 2: 5-(2-(Dimethylamino)ethoxy)-2-ethynyl-N-(1-(naphthalen-1-yl)cyclopropyl)benzamide (Compound 337)

To a solution of 5-(2-(dimethylamino)ethoxy)-N-(1-(naphthalen-1-yl)cyclopropyl)-2-((tri methylsilyl)ethynyl)benzamide (40.0 mg, 84.9 μmol, 1.0 eq) in MeOH (2.0 mL) was added Cs₂CO₃ (138 mg, 425 μmol, 5.0 eq). The resulting mixture was stirred at 20° C. for 1 h. LCMS indicated that the starting material was completely consumed, and the desired mass was detected. The reaction mixture was poured into H₂O (5.0 mL) and extracted with DCM (5.0 mL×4). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 30%-60% B over 8 min; mobile phase A: 0.04% aqueous HCl, mobile phase B: acetonitrile). 5-(2-(Dimethylamino)ethoxy)-2-ethynyl-N-(1-(naphthalen-1-yl)cyclopropyl)benzamide (9.70 mg, 20.6 μmol, 24% yield, HCl salt) was obtained as a yellow solid. M+H⁺=399.1 (LCMS); ¹H NMR (400 MHZ, DMSO-d₆) δ 10.39 (br s, 1H), 9.19 (s, 1H), 8.65 (d, J=8.3 Hz, 1H), 7.93 (d, J=7.6 Hz, 1H), 7.86-7.79 (m, 2H), 7.60-7.41 (m, 4H), 7.01 (dd, J=2.6, 8.6 Hz, 1H), 6.81 (d, J=2.6 Hz, 1H), 4.32 (t, J=4.9 Hz, 2H), 3.89 (s, 1H), 3.43 (br d, J=4.6 Hz, 2H), 2.77 (d, J=4.5 Hz, 6H), 1.44-1.39 (m, 2H), 1.21-1.15 (m, 2H).

### Example 109: 2-Cyclopropyl-5-(2-(dimethylamino)ethoxy)-N-(1-(naphthalen-1-yl)cyclopropyl)benzamide (Compound 353)

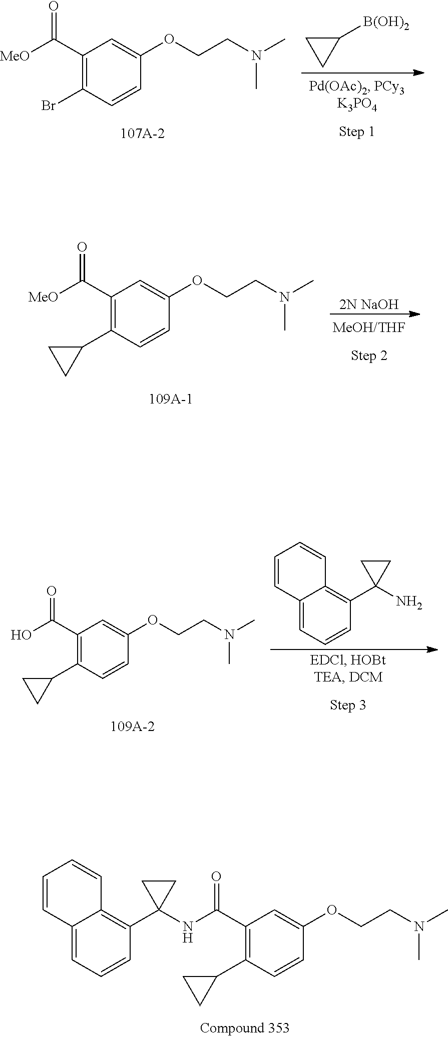

### Step 1: Methyl 2-cyclopropyl-5-(2-(dimethylamino)ethoxy)benzoate (109A-1)

To a solution of methyl 2-bromo-5-(2-(dimethylamino)ethoxy)benzoate (200 mg, 662 μmol, 1.0 eq) and cyclopropylboronic acid (171 mg, 1.99 mmol, 3.0 eq) in a mixture of toluene (10 mL) and H₂O (1.0 mL) were added K₃PO₄ (422 mg, 1.99 mmol, 3.0 eq), PCy₃ (9.28 mg, 33.1 μmol, 10.7 μL, 0.05 eq) and Pd(OAc) 2 (7.43 mg, 33.1 μmol, 0.05 eq). The mixture was stirred at 100° C. for 16 h under a N₂ atmosphere. LCMS indicated that the starting material was completely consumed, and the desired mass was detected. The reaction mixture was allowed to cool to room temperature, poured into H₂O (15 mL) and extracted with EtOAc (10 mL×5). The combined organic layers were dried over Na₂SO₄, filtered, and concentrated under vacuum to give a residue which was purified by preparative TLC (DCM/MeOH=10/1, R_{f}=0.4). Methyl 2-cyclopropyl-5-(2-(dimethylamino)ethoxy)benzoate (100 mg, 380 μmol, 57% yield) was obtained as a yellow oil. M+H⁺=264.1 (LCMS); ¹H NMR (400 MHZ, CDCl₃) δ 7.35 (s, 1H), 6.98 (s, 2H), 4.07 (br t, J=4.9 Hz, 2H), 3.92 (s, 3H), 2.74 (br t, J=4.9 Hz, 2H), 2.58-2.46 (m, 1H), 2.35 (s, 6H), 0.93 (br d, J=8.4 Hz, 2H), 0.61 (br d, J=5.5 Hz, 2H).

### Step 2: 2-Cyclopropyl-5-(2-(dimethylamino)ethoxy)benzoic acid (109A-2)

To a solution of methyl 2-cyclopropyl-5-(2-(dimethylamino)ethoxy)benzoate (90.0 mg, 342 μmol, 1.0 eq) in a mixture of MeOH (5.0 mL) and THF (2.5 mL) was added NaOH (2 M in aqueous, 684 μL, 4.0 eq). The mixture was stirred at 70° C. for 16 h. LCMS indicated that the starting material was completely consumed, and the desired mass was detected. The reaction mixture was allowed to cool to room temperature, poured into H₂O (10 mL) and acidified to pH 6 using HCl (1 M aqueous). The resulting mixture was concentrated under vacuum to remove the water completely. Then the slurry was treated with DCM/MeOH (V/V=10/1, 10 mL) and stirred for 10 min. The mixture was filtered, and the filter cake was washed with DCM/MeOH=10/1 (5.0 mL×2) to ensure all product was washed from the solids. The combined organic layers were concentrated under vacuum to give crude 2-cyclopropyl-5-(2-(dimethylamino)ethoxy)benzoic acid (85 mg) as a yellow gum. M+H⁺=250.1 (LCMS).

### Step 3: 2-Cyclopropyl-5-(2-(dimethylamino)ethoxy)-N-(1-(naphthalen-1-yl)cyclopropyl)benzamide (Compound 353)

To a solution of 2-cyclopropyl-5-(2-(dimethylamino)ethoxy)benzoic acid (70.0 mg, 281 μmol, 1.0 eq) and 1-(naphthalen-1-yl)cyclopropanamine (51.5 mg, 281 μmol, 1.0 eq) in DMF (5.0 mL) were added TEA (85.2 mg, 842 μmol, 117 μL, 3.0 eq), EDCI (64.6 mg, 337 μmol, 1.2 eq) and HOBt (45.5 mg, 337 μmol, 1.2 eq). The mixture was stirred at 20° C. for 16 h. LCMS indicated that the starting material was completely consumed, and the desired mass was detected. The reaction mixture was poured into H₂O (10 mL) and extracted with EtOAc (10 mL×5). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 20%-50% B over 8 min; mobile phase A: 0.04% aqueous HCl, mobile phase B: acetonitrile). 2-Cyclopropyl-5-(2-(dimethylamino)ethoxy)-N-(1-(naphthalen-1-yl)cyclopropyl)benzamide (27.6 mg, 60.5 μmol, 22% yield, HCl salt) was obtained as a yellow solid. M+H⁺=415.1 (LCMS); ¹H NMR (400 MHz, DMSO-d₆) δ 10.21-9.75 (m, 1H), 9.14 (s, 1H), 8.65 (d, J=8.5 Hz, 1H), 7.93 (d, J=8.0 Hz, 1H), 7.86-7.76 (m, 2H), 7.60-7.38 (m, 3H), 6.90-6.78 (m, 2H), 6.63 (d, J=2.1 Hz, 1H), 4.23 (br s, 2H), 3.43 (q, J=4.7 Hz, 2H), 2.79 (d, J=4.6 Hz, 6H), 1.78-1.61 (m, 1H), 1.37 (br s, 2H), 1.19 (br s, 2H), 0.39-0.20 (m, 4H).

### Example 110:3-(2-(Dimethylamino)ethoxy)-2-fluoro-6-methyl-N-(1-(naphthalen-1-yl)cyclopropyl)benzamide (Compound 311)

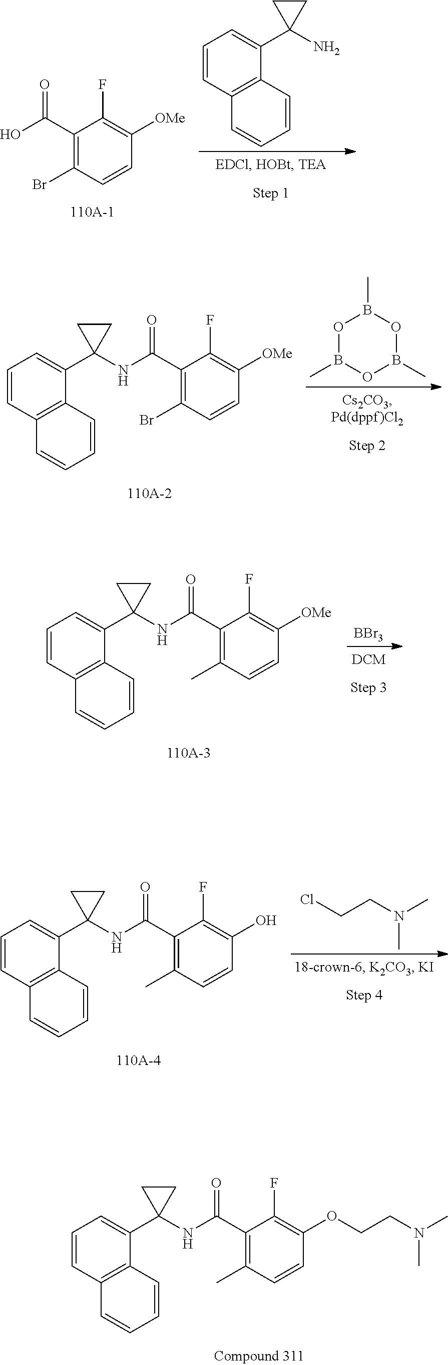

### Step 1: 6-Bromo-2-fluoro-3-methoxy-N-(1-(naphthalen-1-yl)cyclopropyl)benzamide (110A-2)

To a solution of 6-bromo-2-fluoro-3-methoxybenzoic acid (200 mg, 803 μmol, 1.2 eq) and 1-(naphthalen-1-yl)cyclopropanamine (123 mg, 669 μmol, 1.0 eq) in DMF (3.0 mL) were added TEA (203 mg, 2.01 mmol, 279 μL, 3.0 eq), EDCI (154 mg, 803 μmol, 2.5 eq) and HOBt (109 mg, 803 μmol, 1.2 eq). The mixture was stirred at 25° C. for 2 h. LCMS indicated that the starting material was completely consumed, and the desired mass was detected. The reaction mixture was poured into H₂O (5.0 mL) and extracted with DCM (3.0 mL×2). The combined organic layers were dried over Na₂SO₄, filtered, and concentrated under vacuum to give a residue which was purified by flash silica gel chromatography using a gradient of EtOAc/petroleum ether from 0/1 to 1/0. 6-Bromo-2-fluoro-3-methoxy-N-(1-(naphthalen-1-yl)cyclopropyl)benzamide (220 mg, 531 μmol, 79% yield) was obtained as a yellow oil. M+H⁺=414.1 (LCMS).

### Step 2: 2-Fluoro-3-methoxy-6-methyl-N-(1-(naphthalen-1-yl)cyclopropyl)benzamide (110A-3)

To a solution of 6-bromo-2-fluoro-3-methoxy-N-(1-(naphthalen-1-yl)cyclopropyl)benzamide (180 mg, 435 μmol, 1.0 eq) in DMF (3.0 mL) were added 2,4,6-trimethyl-1,3,5,2,4,6-trioxatriborinane (131 mg, 521 μmol, 146 μL, 50% purity in THF, 1.2 eq), Cs₂CO₃ (467 mg, 1.43 mmol, 3.3 eq), and Pd(dppf)Cl₂·CH₂Cl₂ (355 mg, 435 μmol, 1.0 eq). The reaction mixture was degassed and purged with N₂ three times and then stirred at 115° C. for 16 h under a N₂ atmosphere. LCMS indicated that the starting material was completely consumed, and the desired mass was detected. The reaction mixture was allowed to cool to room temperature, poured into H₂O (5.0 mL) and extracted with EtOAc (3.0 mL×2). The combined organic layers were dried over Na₂SO₄, filtered, and concentrated under vacuum to give a residue which was purified by flash silica gel chromatography using a gradient of EtOAc/petroleum ether from 0/1 to 1/1. 2-Fluoro-3-methoxy-6-methyl-N-(1-(naphthalen-1-yl)cyclopropyl)benzamide (220 mg, 531 μmol, 79% yield) was obtained as a yellow oil. M+H⁺=350.1 (LCMS); ¹H NMR (400 MHZ, CDCl₃) δ 8.44 (d, J=8.4 Hz, 1H), 7.95-7.87 (m, 2H), 7.80 (d, J=8.1 Hz, 1H), 7.62-7.41 (m, 6H), 3.80 (s, 3H), 1.95 (s, 3H), 1.61 (br d, J=1.9 Hz, 2H), 1.42-1.36 (m, 2H).

### Step 3: 2-Fluoro-3-hydroxy-6-methyl-N-(1-(naphthalen-1-yl)cyclopropyl)benzamide (110A-4)

To a solution of 2-fluoro-3-methoxy-6-methyl-N-(1-(naphthalen-1-yl)cyclopropyl)benzamide (90.0 mg, 258 μmol, 1.0 eq) in DCM (5.0 mL) was added a solution of BBr₃ (968 mg, 3.86 mmol, 372 μL, 15 eq) in DCM (1.0 mL) dropwise at −78° C. The resulting mixture was stirred at the same temperature for 1 h, warmed to 20° C. and stirred for 16 h under a N₂ atmosphere. LCMS indicated that the starting material was completely consumed, and the desired mass was detected. The reaction mixture was poured into H₂O (5.0 mL) and extracted with DCM (3.0 mL×2). The combined organic layers were dried over Na₂SO₄, filtered, and concentrated under vacuum to give a residue which was purified by flash silica gel chromatography using a gradient of EtOAc/petroleum ether from 0/1 to 1/1. 2-Fluoro-3-hydroxy-6-methyl-N-(1-(naphthalen-1-yl)cyclopropyl)benzamide (40.0 mg, 119 μmol, 46% yield) was obtained as a yellow oil. M+H⁺=336.1 (LCMS).

### Step 4:3-(2-(Dimethylamino)ethoxy)-2-fluoro-6-methyl-N-(1-(naphthalen-1-yl)cyclopropyl)benzamide (Compound 311)

To a solution of 2-fluoro-3-hydroxy-6-methyl-N-(1-(naphthalen-1-yl)cyclopropyl)benzamide (30.0 mg, 890 μmol, 1.0 eq) in DMF (3.0 mL) was added K₂CO₃ (61.8 mg, 447 μmol, 5.0 eq). The mixture was stirred at 20° C. for 30 min. To the reaction mixture were added 2-chloro-N,N-dimethylethanamine (12.9 mg, 89.5 μmol, 1.0 eq, HCl salt), 18-crown-6 (37.8 mg, 143 μmol, 1.6 eq) and KI (25.2 mg, 152 μmol, 1.7 eq). The mixture was stirred at 70° C. for 16 h. LCMS indicated that the starting material was completely consumed, and the desired mass was detected. The reaction mixture was allowed to cool to room temperature, poured into H₂O (5.0 mL) and extracted with EtOAc (3.0 mL×2). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 1%-50% B over 8 min; mobile phase A: 0.2% aqueous FA, mobile phase B: acetonitrile). 3-(2-(Dimethylamino)ethoxy)-2-fluoro-6-methyl-N-(1-(naphthalen-1-yl)cyclopropyl)benzamide (4.10 mg, 8.67 μmol, 10% yield, FA salt) was obtained as a white solid. M+H⁺=407.1 (LCMS); ¹H NMR (400 MHZ, DMSO-d₆) δ 9.35 (s, 1H), 8.56 (d, J=8.3 Hz, 1H), 8.19 (s, 1H), 7.96-7.89 (m, 1H), 7.87-7.76 (m, 2H), 7.62-7.40 (m, 3H), 7.02 (t, J=8.6 Hz, 1H), 6.84 (d, J=8.4 Hz, 1H), 4.03 (t, J=5.7 Hz, 2H), 2.57 (t, J=5.7 Hz, 2H), 2.18 (s, 6H), 1.78 (s, 3H), 1.31 (br s, 2H), 1.21-1.14 (m, 2H).

### Example 111: 5-Amino-4-iodo-2-methyl-N-(1-(naphthalen-1-yl)cyclopropyl)benzamide (Compound 216)

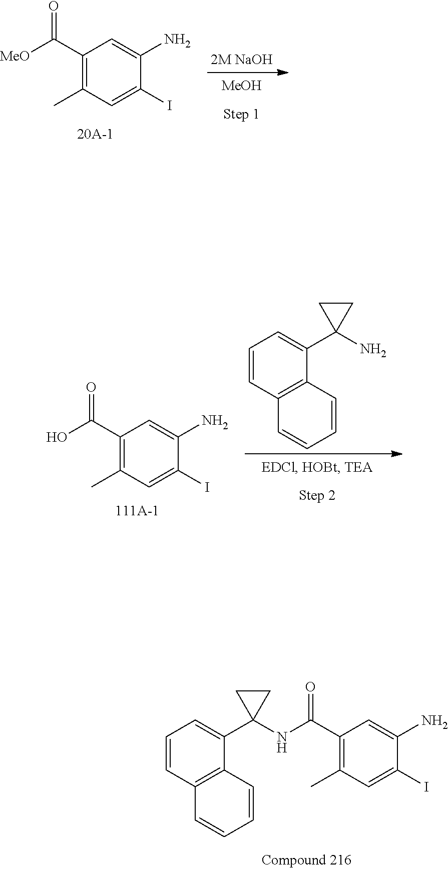 [SMILES: "MeOH" ; COC(=O)c1cc(N)c(I)cc1C ; "2M NaOH" ; Cc1cc(I)c(N)cc1C(=O)O ; NC1(c2cccc3ccccc23)CC1 ; Cc1cc(I)c(N)cc1C(=O)NC1(c2cccc3ccccc23)CC1]

### Step 1: 5-Amino-4-iodo-2-methylbenzoic acid (111A-1)

To a solution of methyl 5-amino-4-iodo-2-methylbenzoate (200 mg, 687 μmol, 1.0 eq) in MeOH (10 mL) was added NaOH (2 M in aqueous, 1.03 mL, 3.0 eq). The mixture was stirred at 25° C. for 2 h and then was stirred another 2 h at 70° C. LCMS indicated that the starting material was completely consumed, and the desired mass was detected. The reaction mixture was poured into H₂O (10 mL) and acidified to pH 6 with HCl (1 M aqueous), and a precipitate was formed. The mixture was filtered and the solid was washed with H₂O (5.0 mL), dried under vacuum to give 5-amino-4-iodo-2-methylbenzoic acid (180 mg, 650 μmol, 95% yield) as a white solid, which was used in the next step without any further purification. M+H⁺=278.0 (LCMS).

### Step 2: 5-Amino-4-iodo-2-methyl-N-(1-(naphthalen-1-yl)cyclopropyl)benzamide (Compound 216)

To a solution of 5-amino-4-iodo-2-methylbenzoic acid (130 mg, 469 μmol, 1.0 eq) and 1-(naphthalen-1-yl)cyclopropanamine (86.0 mg, 469 μmol, 1.0 eq) in DMF (7.0 mL) were added TEA (142 mg, 1.41 mmol, 196 μL, 3.0 eq), EDCI (180 mg, 938 μmol, 2.0 eq) and HOBt (127 mg, 938 μmol, 2.0 eq). The mixture was stirred at 25° C. for 3 h. LCMS indicated that the starting material was completely consumed, and the desired mass was detected. The reaction mixture was poured into H₂O (10 mL), and a precipitate was formed. The mixture was filtered, and the filter cake was washed with H₂O (5.0 mL), dried under vacuum to give a residue which was purified by preparative HPLC (Waters Xbridge BEH C18 column (100×30 mm, 10 μm); flow rate: 25 mL/min; gradient: 35%-65% B over 8 min; mobile phase A: 10 mM aqueous NH₄HCO₃, mobile phase B: acetonitrile). 5-Amino-4-iodo-2-methyl-N-(1-(naphthalen-1-yl)cyclopropyl)benzamide (28.4 mg, 64.2 μmol, 20% yield) was obtained as a white solid. M+H⁺=442.9 (LCMS); ¹H NMR (400 MHZ, CDCl₃) δ 8.44 (d, J=8.4 Hz, 1H), 8.00-7.87 (m, 2H), 7.81 (d, J=8.3 Hz, 1H), 7.65-7.44 (m, 3H), 7.39 (s, 1H), 6.49 (s, 1H), 6.44 (br s, 1H), 3.91 (br s, 2H), 2.03 (s, 3H), 1.56-1.52 (m, 2H), 1.42-1.34 (m, 2H).

### Example 112:4-Bromo-5-(2-(dimethylamino)ethoxy)-2-methyl-N-(1-(naphthalen-1-yl)cyclopropyl)benzamide (Compound 369)

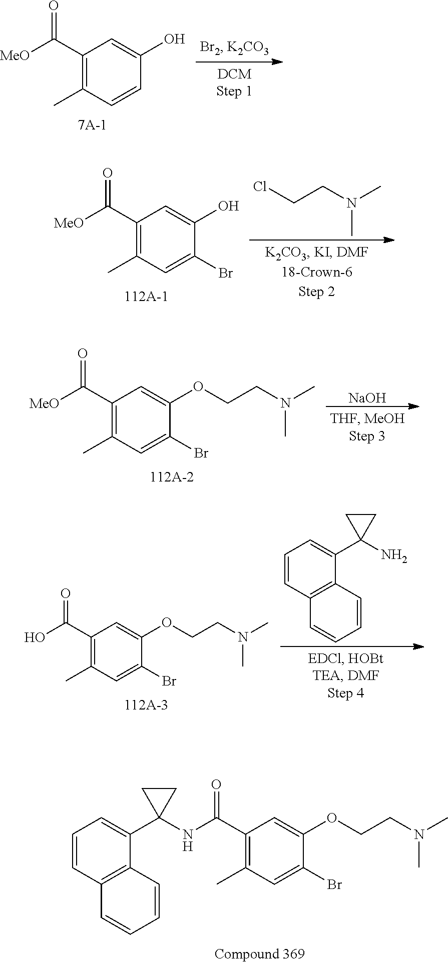

### Step 1: Methyl 4-bromo-5-hydroxy-2-methylbenzoate (112A-1)

To a solution of methyl 5-hydroxy-2-methylbenzoate (200 mg, 1.20 mmol, 1.0 eq) in DCM (2.0 mL) was added K₂CO₃ (175 mg, 1.26 mmol, 1.05 eq) at 25° C. The mixture was degassed and purged with N₂ three times. To this mixture was added a solution of Br₂ (192 mg, 1.20 mmol, 62.0 μL, 1.0 eq) in DCM (4.0 mL) at −15° C. dropwise. After the addition was completed, the mixture was stirred at 25° C. for 16 h under a N₂ atmosphere. TLC indicated that the starting material was completely consumed, and a main spot was detected. The reaction mixture was poured into H₂O (6.0 mL) and extracted with DCM (4.0 mL×3). The combined organic layers were dried over Na₂SO₄, filtered, and concentrated under vacuum to give a residue which was purified by was purified by preparative TLC (EtOAc/petroleum ether=1/3, R_{f}=0.6). Methyl 4-bromo-5-hydroxy-2-methylbenzoate (160 mg, 54% yield) was obtained as a yellow solid.

### Step 2: Methyl 4-bromo-5-(2-(dimethylamino)ethoxy)-2-methylbenzoate (112A-2)

To a solution of 2-chloro-N,N-dimethylethanamine (188 mg, 1.31 mmol, 4.0 eq) in DMF (5.0 mL) was added K₂CO₃ (902 mg, 6.53 mmol, 20 eq) and the reaction mixture was stirred at 20° C. for 30 min. Methyl 4-bromo-5-hydroxy-2-methylbenzoate (80.0 mg, 326 μmol, 1.0 eq), 18-crown-6 (545 mg, 2.06 mmol, 6.3 eq) and KI (364 mg, 2.19 mmol, 6.7 eq) were added in sequence. The resulting mixture was stirred at 70° C. for 11 h. LCMS indicated that the starting material was completely consumed, and the desired mass was detected. The reaction mixture was poured into H₂O (10 mL) and extracted with EtOAc (4.0 mL×3). The combined organic layers were dried over Na₂SO₄, filtered, and concentrated under vacuum to give a residue which was purified by preparative HPLC (Phenomenex Water Xbridge BEH C18 (100×30 mm, 10 μm); flow rate: 25 mL/min; gradient: 30%-50% B over 8 min; mobile phase A: 10 mM aqueous NH₄HCO₃, mobile phase B: acetonitrile). Methyl 4-bromo-5-(2-(dimethylamino)ethoxy)-2-methylbenzoate (88.0 mg, 273 μmol, 84% yield) was obtained as a yellow solid. M+H⁺=316.0 (LCMS); ¹H NMR (400 MHz, CDCl₃) δ 7.38 (s, 1H), 7.37 (s, 1H), 4.09 (s, 2H), 3.82 (s, 3H), 2.76 (s, 2H), 2.43 (s, 3H), 2.33 (s, 6H).

### Step 3:4-Bromo-5-(2-(dimethylamino)ethoxy)-2-methylbenzoic acid (112A-3)

To a solution of methyl 4-bromo-5-(2-(dimethylamino)ethoxy)-2-methylbenzoate (68.0 mg, 215 μmol, 1.0 eq) in a mixture of MeOH (2.7 mL) and THF (1.4 mL) was added NaOH (2 M aqueous, 1.4 mL, 12.7 eq). The mixture was stirred at 60° C. for 16 h. LCMS indicated that the starting material was completely consumed, and the desired mass was detected. The reaction mixture was poured into H₂O (3.0 mL) and extracted with MTBE (2.0 mL×2). The aqueous layer was acidified to pH 6 using HCl (1 M aqueous). The mixture was extracted with EtOAc (3.0 mL×3). The combined organic layers were dried over Na₂SO₄, filtered, and concentrated under vacuum to give the product 4-bromo-5-(2-(dimethylamino)ethoxy)-2-methylbenzoic acid (60.0 mg, 199 μmol, 92% yield) as a yellow solid. M+H⁺=302.0 (LCMS); ¹H NMR (400 MHZ, CDCl₃) δ 7.53 (s, 1H), 7.38 (s, 1H), 4.52-4.38 (m, 2H), 3.36-3.24 (m, 2H), 2.78 (s, 6H), 2.53 (s, 3H).

### Step 4:4-Bromo-5-(2-(dimethylamino)ethoxy)-2-methyl-N-(1-(naphthalen-1-yl)cyclopropyl)benzamide (Compound 369)

To a solution of 4-bromo-5-(2-(dimethylamino)ethoxy)-2-methylbenzoic acid (50.0 mg, 165 μmol, 1.0 eq) and 1-(naphthalen-1-yl)cyclopropanamine (24.3 mg, 132 μmol, 0.8 eq) in DMF (2.5 mL) were added TEA (33.5 mg, 331 μmol, 46.1 μL, 2.0 eq), EDCI (47.6 mg, 248 μmol, 1.5 eq) and HOBt (33.5 mg, 331 μmol, 2.0 eq). The mixture was stirred at 20° C. for 16 h. LCMS indicated that the starting material was completely consumed, and the desired mass was detected. The reaction mixture was poured into H₂O (3.0 mL) and extracted with EtOAc (3.0 mL×3). The combined organic layers were dried over Na₂SO₄, filtered, and concentrated under vacuum to give a residue which was purified by preparative HPLC (Phenomenex Luna (80×30 mm, 3 μm); flow rate: 25 mL/min; gradient: 1%-40% B over 8 min; mobile phase A: 0.04% aqueous HCl, mobile phase B: acetonitrile). 4-Bromo-5-(2-(dimethylamino)ethoxy)-2-methyl-N-(1-(naphthalen-1-yl)cyclopropyl)benzamide (15.6 mg, 30.9 μmol, 19% yield, HCl salt) was obtained as a white solid. M+H⁺=467.0 (LCMS); ¹H NMR (400 MHZ, CDCl₃) δ 13.08-12.84 (m, 1H), 8.67-8.57 (m, 1H), 8.01-7.92 (m, 1H), 7.92-7.86 (m, 1H), 7.83-7.76 (m, 1H), 7.67-7.58 (m, 1H), 7.54-7.43 (m, 2H), 7.01 (s, 1H), 6.87 (br s, 1H), 4.51 (br d, J=1.4 Hz, 2H), 3.59-3.24 (m, 2H), 2.95 (br s, 6H), 2.10 (s, 3H), 1.64-1.55 (m, 2H), 1.45-1.35 (m, 2H).

### Example 113: 5-(2-Aminoethoxy)-2-methyl-N-(1-(naphthalen-1-yl)cyclopropyl)benzamide (Compound 366)

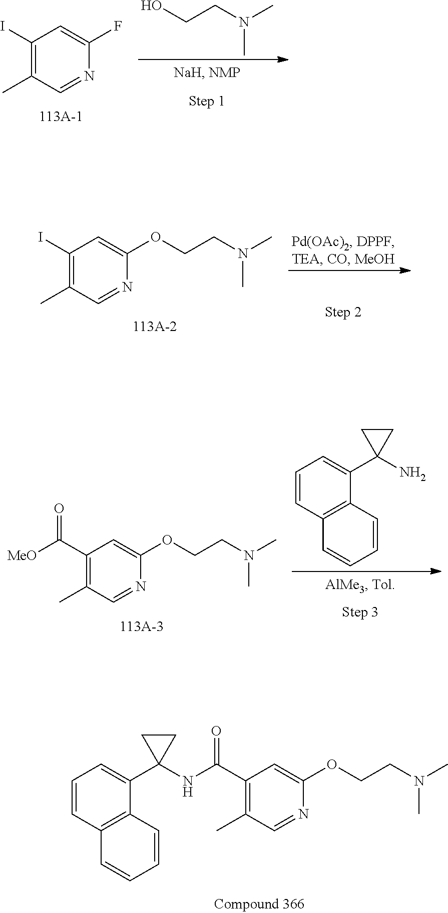

### Step 1: 2-((4-Iodo-5-methylpyridin-2-yl)oxy)-N,N-dimethylethanamine (113A-2)

To a solution of 2-(dimethylamino) ethanol (1.69 g, 19.0 mmol, 3.0 eq) in NMP (10 mL) was added NaH (759 mg, 19.0 mmol, 60% purity, 3.0 eq) at 0° C., the reaction mixture was stirred at 25° C. for 30 min. 2-Fluoro-4-iodo-5-methyl-pyridine (1.50 g, 6.33 mmol, 1.0 eq) was added to the solution, and the reaction mixture was stirred at 25° C. for 1 h. LCMS indicated that the starting material was completely consumed, and the desired mass was detected. The reaction mixture was poured into H₂O (30 mL) and extracted with EtOAc (50 mL×3). The combined organic layers were dried over Na₂SO₄, filtered, and concentrated under vacuum to give a residue which was purified by preparative HPLC (Phenomenex C18 (75×30 mm, 3 μm); flow rate: 25 mL/min; gradient: 25%-75% B over 8 min; mobile phase A: 10 mM aqueous NH₄HCO₃, mobile phase B: acetonitrile) to give 2-((4-iodo-5-methylpyridin-2-yl)oxy)-N,N-dimethylethanamine (600 mg, 2.00 mmol, 31% yield) as a white oil. M+H⁺=307.0 (LCMS).

### Step 2: Methyl 2-(2-(dimethylamino)ethoxy)-5-methylisonicotinate (113A-3)

A mixture of 2-[(4-iodo-5-methyl-2-pyridyl)oxy]-N,N-dimethyl-ethanamine (400 mg, 1.31 mmol, 1.0 eq), Pd(OAc) 2 (29.3 mg, 131 μmol, 0.1 eq), DPPF (72.4 mg, 131 μmol, 0.1 eq) and TEA (661 mg, 6.53 mmol, 909 μL, 5.0 eq) in MeOH (10 mL) was stirred at 80° C. for 16 h under a CO (50 psi) atmosphere. LCMS indicated that the starting material was completely consumed, and the desired mass was detected. The reaction mixture was allowed to cool to room temperature, filtered, and the filtrate was concentrated under vacuum to give a residue which was purified by preparative TLC ((EtOAc/petroleum ether=1/1, R_{f}=0.4). Methyl 2-[2-(dimethylamino)ethoxy]-5-methyl-pyridine-4-carboxylate (300 mg, 1.00 mmol, 96% yield) was obtained as a brown oil. M+H⁺=239.1 (LCMS).

### Step 3: 2-(2-(Dimethylamino)ethoxy)-5-methyl-N-(1-(naphthalen-1-yl)cyclopropyl) isonicotinamide (Compound 366)

To a mixture of methyl 2-[2-(dimethylamino)ethoxy]-5-methyl-pyridine-4-carboxylate (100 mg, 400 μmol, 1.0 eq) and 1-(1-naphthyl)cyclopropanamine (76.9 mg, 400 μmol, 1.0 eq) in toluene (2.0 mL) was added AlMe₃ (2 M in toluene, 600 μL, 3.0 eq) at 0° C. The reaction mixture was stirred at 100° C. for 1 h under a N₂ atmosphere. LCMS indicated that the starting material was completely consumed, and the desired mass was detected. The reaction mixture was allowed to cool to room temperature, poured into H₂O (100 μL) and TFA (100 μL), then the reaction mixture was concentrated under vacuum to give a residue which was purified by preparative HPLC (Phenomenex Gemini C18 column (150×30 mm, 5 μm); flow rate: 25 mL/min; gradient: 10%-55% B over 8 min; mobile phase A: 0.1% aqueous TFA, mobile phase B: acetonitrile). 2-[2-(Dimethylamino)ethoxy]-5-methyl-N-[1-(1-naphthyl)cyclopropyl]pyridine-4-carboxamide (48.8 mg, 69.7 μmol, 23% yield, TFA salt) was obtained as a white solid. M+H⁺=390.1 (LCMS); ¹H NMR (400 MHZ, DMSO-d₆) δ 9.52 (br d, J=4.0 Hz, 1H), 9.35 (s, 1H), 8.60 (d, J=8.4 Hz, 1H), 8.01-7.90 (m, 2H), 7.83 (dd, J=7.6, 15.6 Hz, 2H), 7.62-7.40 (m, 3H), 6.52 (s, 1H), 4.55-4.35 (m, 2H), 3.44 (br s, 2H), 2.81 (br s, 6H), 1.93 (s, 3H), 1.39-1.15 (m, 4H).

### Example 114: 2-(2-(Dimethylamino)ethoxy)-5-methyl-N-(1-(naphthalen-1-yl)cyclopropyl)pyrimidine-4-carboxamide (Compound 350)

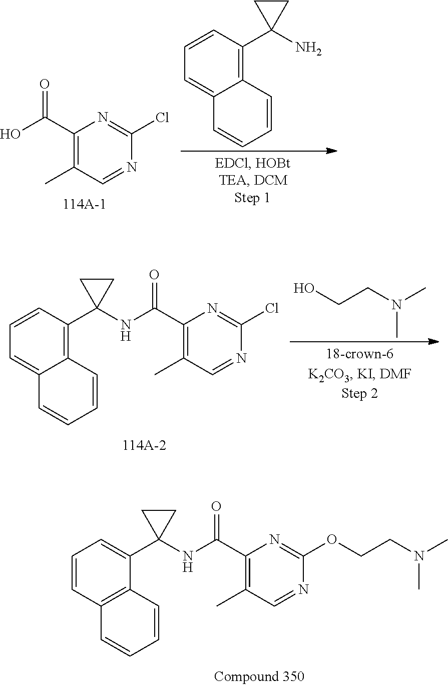

### Step 1: 2-Chloro-5-methyl-N-(1-(naphthalen-1-yl)cyclopropyl)pyrimidine-4-carboxamide (114A-2)

To a solution of 2-chloro-5-methylpyrimidine-4-carboxylic acid (150 mg, 869 μmol, 1.0 eq) and 1-(naphthalen-1-yl)cyclopropanamine (143 mg, 782 μmol, 0.8 eq) in DCM (5.0 mL) were added TEA (264 mg, 2.61 mmol, 363 μL, 3.0 eq), EDCI (200 mg, 1.04 mmol, 1.2 eq) and HOBt (141 mg, 1.04 mmol, 1.2 eq). The mixture was stirred at 20° C. for 16 h. TLC indicated that the starting material was completely consumed. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative TLC (EtOAc/petroleum ether=1/1, R_{f}=0.8). 2-Chloro-5-methyl-N-(1-(naphthalen-1-yl)cyclopropyl)pyrimidine-4-carboxamide (60.0 mg, 178 μmol, 20% yield) was obtained as a yellow oil. M+H⁺=338.1 (LCMS).

### Step 2: 2-(2-(Dimethylamino)ethoxy)-5-methyl-N-(1-(naphthalen-1-yl)cyclopropyl)pyrimidine-4-carboxamide (Compound 350)

To a solution of 2-chloro-5-methyl-N-(1-(naphthalen-1-yl)cyclopropyl)pyrimidine-4-carboxamide (100 mg, 296 μmol, 1.0 eq) and 2-(dimethylamino) ethanol (26.4 mg, 210 μmol, 29.7 μL, 0.7 eq, HCl salt) in DMF (5.0 mL) were added 18-crown-6 (124 mg, 468 μmol, 1.6 eq), K₂CO₃ (205 mg, 1.48 mmol, 5.0 eq) and KI (82.6 mg, 497 μmol, 1.7 eq). The mixture was stirred at 70° C. for 16 h. LCMS indicated that the starting material was completely consumed, and the desired mass was detected. The reaction mixture was allowed to cool to room temperature, poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 20%-60% B over 8 min; mobile phase A: 0.2% aqueous FA, mobile phase B: acetonitrile). 2-(2-(Dimethylamino)ethoxy)-5-methyl-N-(1-(naphthalen-1-yl)cyclopropyl)pyrimidine-4-carboxamide (29.1 mg, 74.0 μmol, 25% yield, FA salt) was obtained as a white solid. M+H⁺=391.1 (LCMS); ¹H NMR (400 MHZ, DMSO-d₆) δ 9.45 (s, 1H), 8.68 (d, J=8.3 Hz, 1H), 8.47 (s, 1H), 7.93 (d, J=7.9 Hz, 1H), 7.87-7.80 (m, 2H), 7.62-7.43 (m, 3H), 4.33 (t, J=5.7 Hz, 2H), 2.54 (s, 2H), 2.16-2.11 (m, 9H), 1.41 (s, 2H), 1.21 (br s, 2H).

### Example 115: 5-(2-(Dimethylamino)ethoxy)-2-methyl-N-(1-(naphthalen-1-yl)cyclopropyl) nicotinamide (Compound 275)

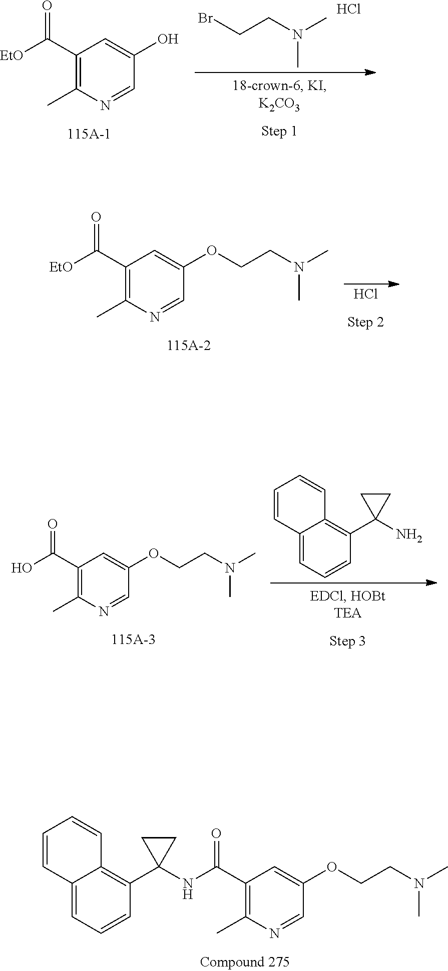

### Step 1: Ethyl 5-(2-(dimethylamino)ethoxy)-2-methylnicotinate (115A-2)

A mixture of ethyl 5-hydroxy-2-methylnicotinate (300 mg, 1.66 mmol, 1.0 eq) and 2-bromo-N,N-dimethylethanamine (262 mg, 1.82 mmol, 1.1 eq, HCl) in DMF (10 mL) was degassed and purged with N₂ three times. To the mixture were added 18-crown-6 (691 mg, 2.62 mmol, 1.6 eq), KI (462 mg, 2.78 mmol, 1.7 eq) and K₂CO₃ (1.1 g, 8.28 mmol, 5.0 eq). The resulting mixture was stirred at 70° C. for 14 h under a N₂ atmosphere. LCMS indicated that starting material was completely consumed, and the desired mass was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by flash silica gel chromatography using a gradient of DCM/MeOH from 200/1 to 10/1. Ethyl 5-(2-(dimethylamino)ethoxy)-2-methylnicotinate (200 mg, 380 μmol, 48% yield) was obtained as a white solid. M+H⁺=253.2 (LCMS); ¹H NMR (400 MHZ, CDCl₃) δ 8.35 (d, J=3.0 Hz, 1H), 7.74 (d, J=3.0 Hz, 1H), 4.39 (q, J=7.1 Hz, 2H), 4.22-4.12 (m, 2H), 2.76 (s, 3H), 2.42 (br s, 6H), 1.41 (t, J=7.1 Hz, 3H).

### Step 2: 5-(2-(Dimethylamino)ethoxy)-2-methylnicotinic acid (115A-3)

A solution of ethyl 5-(2-(dimethylamino)ethoxy)-2-methylnicotinate (160 mg, 634 μmol, 1.0 eq) in HCl (2 M aqueous, 5.0 mL) was stirred at 100° C. for 14 h. LCMS indicated that starting material was completely consumed, and the desired mass was detected. The mixture was allowed to cool to room temperature and concentrated under vacuum to give the crude product 5-(2-(dimethylamino)ethoxy)-2-methylnicotinic acid (160 mg), which was used in the next step without any further purification. M+H⁺=225.2 (LCMS).

### Step 3: 5-(2-(Dimethylamino)ethoxy)-2-methyl-N-(1-(naphthalen-1-yl)cyclopropyl) nicotinamide (Compound 275)

To a solution of 5-(2-(dimethylamino)ethoxy)-2-methylnicotinic acid (100 mg, 446 μmol, 1.0 eq) and 1-(naphthalen-1-yl)cyclopropanamine (82.0 mg, 446 μmol, 1.0 eq) in DCM (6.0 mL) were added TEA (135 mg, 1.34 mmol, 186 μL, 3.0 eq), EDCI (128 mg, 669 μmol, 1.5 eq) and HOBt (90.0 mg, 669 μmol, 1.5 eq). The mixture was stirred at 20° C. for 18 h. LCMS indicated that starting material was completely consumed, and the desired mass was detected. The reaction mixture was poured into H₂O (5.0 mL) and extracted with DCM (5.0 mL×2). The combined organic layers were dried over Na₂SO₄, filtered, and concentrated under vacuum to give a residue which was purified by preparative HPLC (Phenomenex Gemini C18 column (75×30 mm, 3 μm); flow rate: 25 mL/min; gradient: 1%-35% B over 8 min; mobile phase A: 0.2% aqueous FA, mobile phase B: acetonitrile). 5-(2-(Dimethylamino)ethoxy)-2-methyl-N-(1-(naphthalen-1-yl)cyclopropyl) nicotinamide (60.0 mg, 155 μmol, 35% yield, FA salt) was obtained as a white solid. M+H⁺=390.1 (LCMS); ¹H NMR (400 MHZ, DMSO-d₆) δ 9.13 (s, 1H), 8.53 (d, J=8.4 Hz, 1H), 8.04 (d, J=2.9 Hz, 1H), 7.83 (d, J=7.9 Hz, 1H), 7.73 (t, J=7.3 Hz, 2H), 7.51-7.32 (m, 3H), 6.97 (d, J=3.0 Hz, 1H), 3.94 (t, J=5.7 Hz, 2H), 2.43-2.37 (m, 3H), 2.08 (s, 6H), 2.02 (s, 3H), 1.33-1.24 (m, 2H), 1.13-1.05 (m, 2H).

### Example 116: 5-Methyl-N-(1-(naphthalen-1-yl)cyclopropyl)-1H-indole-6-carboxamide (Compound 199)

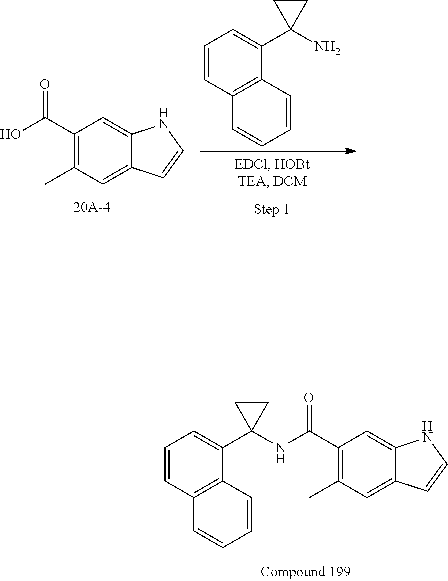 [SMILES: Cc1cc2cc[nH]c2cc1C(=O)O ; NC1(c2cccc3ccccc23)CC1 ; Cc1cc2cc[nH]c2cc1C(=O)NC1(c2cccc3ccccc23)CC1]

### Step 1: 5-Methyl-N-(1-(naphthalen-1-yl)cyclopropyl)-1H-indole-6-carboxamide (Compound 199)

To a solution of 5-methyl-1H-indole-6-carboxylic acid (140 mg, 800 μmol, 1.0 eq) and 1-(naphthalen-1-yl)cyclopropanamine (146 mg, 799 μmol, 1.0 eq) in DCM (4.0 mL) were added TEA (243 mg, 2.40 mmol, 300 μL, 3.0 eq), EDCI (383 mg, 2.00 mmol, 2.5 eq) and HOBt (267 mg, 2.00 mmol, 2.5 eq). The resulting mixture was stirred at 20° C. for 16 h. LCMS indicated that the starting material was completely consumed, and the desired mass was detected. The reaction mixture was poured into H₂O (5.0 mL) and extracted with DCM (2.0 mL×3). The combined organic layers were dried over Na₂SO₄, filtered, and concentrated under vacuum to give a residue which was purified by preparative HPLC (Phenomenex Gemini C18 column (80×30 mm, 3 μm); flow rate: 25 mL/min; gradient: 30%-60% B over 8 min; mobile phase A: 0.04% aqueous HCl, mobile phase B: acetonitrile). 5-Methyl-N-(1-(naphthalen-1-yl)cyclopropyl)-1H-indole-6-carboxamide (200 mg, 512 μmol, 64% yield. HCl salt) was obtained as a white solid. M+H⁺=341.1 (LCMS); ¹H NMR (400 MHZ, DMSO-d₆) δ 11.20-10.85 (m, 1H), 9.02 (s, 1H), 8.82-8.61 (m, 1H), 7.99-7.90 (m, 1H), 7.87-7.80 (m, 2H), 7.64-7.42 (m, 3H), 7.34-7.29 (m, 1H), 7.26-7.23 (m, 1H), 7.11 (s, 1H), 6.29 (br s, 1H), 2.22-2.10 (m, 3H), 1.44-1.31 (m, 2H), 1.23-1.10 (m, 2H).

### Example 117: 2-(Methoxymethyl)-5-methyl-N-(1-(naphthalen-1-yl)cyclopropyl)-1H-indole-6-carboxamide (Compound 240)

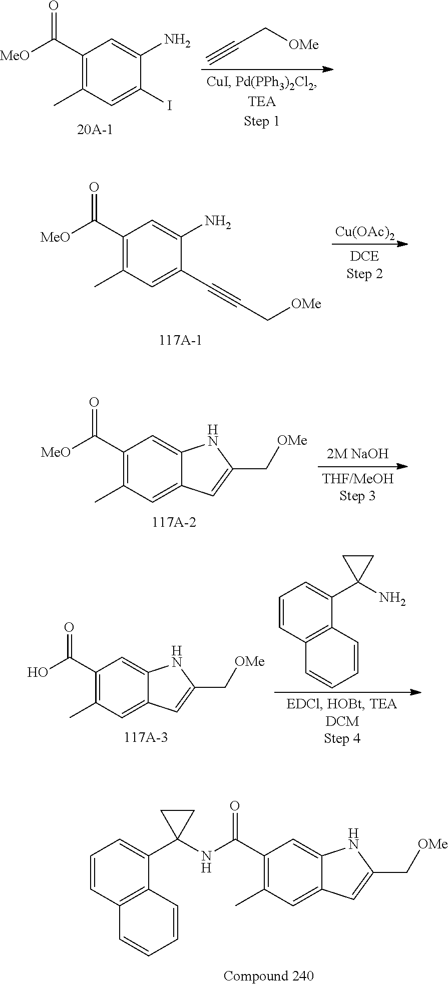

### Step 1: Methyl 5-amino-4-(3-methoxyprop-1-yn-1-yl)-2-methylbenzoate (117A-1)

To a solution of methyl 5-amino-4-iodo-2-methylbenzoate (500 mg, 1.72 mmol, 1.0 eq) in TEA (10 mL) were added 3-methoxyprop-1-yne (120 mg, 1.72 mmol, 1.0 eq), Pd(PPh₃)₂Cl₂ (120 mg, 172 μmol, 0.1 eq) and CuI (16.4 mg, 85.9 μmol, 0.1 eq). The mixture was stirred at 20° C. for 16 h under a N₂ atmosphere. TLC indicated that the starting material was completely consumed. The reaction mixture was poured into H₂O (10 mL) and extracted with EtOAc (4.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/10. Methyl 5-amino-4-(3-methoxyprop-1-yn-1-yl)-2-methylbenzoate (400 mg, 1.54 mmol, 90% yield) was obtained as a yellow oil. M+H⁺=234.1 (LCMS); ¹H NMR (400 MHZ, CDCl₃) δ 7.36-7.34 (m, 1H), 7.23 (s, 1H), 4.45 (s, 2H), 3.94 (s, 3H), 3.53 (s, 3H), 2.53-2.48 (m, 3H).

### Step 2: Methyl 2-(methoxymethyl)-5-methyl-1H-indole-6-carboxylate (117A-2)

To a solution of methyl 5-amino-4-(3-methoxyprop-1-yn-1-yl)-2-methylbenzoate (350 mg, 1.50 mmol, 1.0 eq) in DCE (6.0 mL) was added Cu(OAc) 2 (545 mg, 3.00 mmol, 2.0 eq). The mixture was degassed and purged with N₂ three times and stirred at 130° C. for 2 h under a N₂ atmosphere. TLC indicated that the starting material was completely consumed. The reaction mixture was allowed to cool to room temperature, filtered and concentrated under vacuum to give a residue which was purified by preparative TLC (EtOAc/petroleum ether=1/1, R_{f}=0.6). Methyl 2-(methoxymethyl)-5-methyl-1H-indole-6-carboxylate (180 mg, 772 μmol, 51% yield) was obtained as a yellow oil. M+H⁺=234.1 (LCMS); ¹H NMR (400 MHZ, CDCl₃) δ 8.41 (br s, 1H), 8.03 (s, 1H), 7.41 (s, 1H), 6.37 (s, 1H), 4.63 (s, 2H), 3.91 (s, 3H), 3.41 (s, 3H), 2.68 (s, 3H).

### Step 3: 2-(Methoxymethyl)-5-methyl-1H-indole-6-carboxylic acid (117A-3)

To a solution of methyl 2-(methoxymethyl)-5-methyl-1H-indole-6-carboxylate (90.0 mg, 386 μmol, 1.0 eq) in a mixture of MeOH (4.0 mL) and THF (2.0 mL) was added NaOH (2 M, 1.0 mL, 5.2 eq). The mixture was stirred at 20° C. for 2 h. LCMS indicated that the starting material was completely consumed, and the desired mass was detected. The reaction mixture was poured into H₂O (6.0 mL) and extracted with MTBE (2.0 mL×3). The aqueous layer was basified to pH 4 using HCl (1 M aqueous) and extracted with EtOAc (3.0 mL×2). The combined organic layers were dried over Na₂SO₄, filtered, and concentrated under vacuum to give the crude product 2-(methoxymethyl)-5-methyl-1H-indole-6-carboxylic acid (60.0 mg), which was used in the next step without any further purification. M+H⁺=220.0 (LCMS).

### Step 4: 2-(Methoxymethyl)-5-methyl-N-(1-(naphthalen-1-yl)cyclopropyl)-1H-indole-6-carboxamide (Compound 240)

To a solution of 2-(methoxymethyl)-5-methyl-1H-indole-6-carboxylic acid (60.0 mg, 274 μmol, 1.0 eq) and 1-(naphthalen-1-yl)cyclopropanamine (50.2 mg, 274 μmol, 1.0 eq) in DCM (5.0 mL) were added TEA (83.1 mg, 821 μmol, 114 μL, 3.0 eq), EDCI (78.7 mg, 411 μmol, 1.5 eq) and HOBt (55.5 mg, 411 μmol, 1.5 eq). The mixture was stirred at 20° C. for 2 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 40%-70% B over 8 min; mobile phase A: 0.04% aqueous HCl, mobile phase B: acetonitrile). 2-(Methoxymethyl)-5-methyl-N-(1-(naphthalen-1-yl)cyclopropyl)-1H-indole-6-carboxamide (5.20 mg, 12.3 μmol, 5% yield, HCl salt) was obtained as a white solid. M+H⁺=385.1 (LCMS); ¹H NMR (400 MHZ, DMSO-d₆) δ 11.23-10.99 (m, 1H), 9.02 (s, 1H), 8.70 (d, J=8.9 Hz, 1H), 7.94 (d, J=7.8 Hz, 1H), 7.83 (d, J=7.7 Hz, 2H), 7.61-7.43 (m, 3H), 7.20 (s, 1H), 7.04 (s, 1H), 6.24 (s, 1H), 4.47 (s, 2H), 3.23 (s, 3H), 2.13 (s, 3H), 1.36 (s, 2H), 1.17 (br d, J=1.1 Hz, 2H).

### Example 118: tert-Butyl((5-methyl-6-((1-(naphthalen-1-yl)cyclopropyl)carbamoyl)-1H-indol-2-yl)methyl)carbamate (Compound 180)

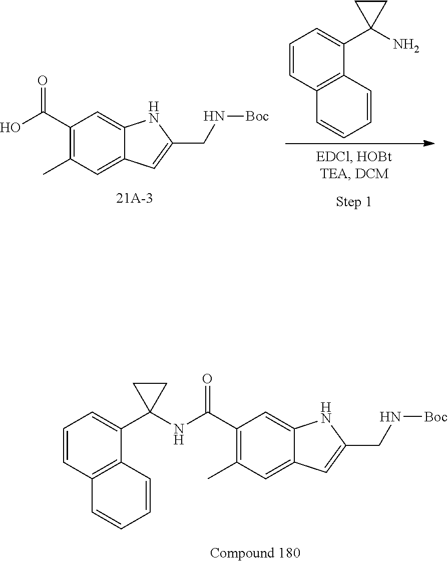

### Step 1: tert-Butyl((5-methyl-6-((1-(naphthalen-1-yl)cyclopropyl)carbamoyl)-1H-indol-2-yl)methyl)carbamate (Compound 180)

To a solution of 2-(((tert-butoxycarbonyl)amino)methyl)-5-methyl-1H-indole-6-carboxylic acid (210 mg, 690 μmol, 1.0 eq) and 1-(naphthalen-1-yl)cyclopropanamine (139 mg, 759 μmol, 1.1 eq) in DCM (5.0 mL) was added TEA (209 mg, 2.07 mmol, 288 μL, 3.0 eq), followed by HOBt (140 mg, 1.04 mmol, 1.5 eq) and EDCI (198 mg, 1.04 mmol, 1.5 eq). The mixture was stirred at 20° C. for 15 h. LCMS indicated that the starting material was completely consumed, and the desired mass was detected. The reaction mixture was poured into H₂O (10 mL) and extracted with DCM (8.0 mL×5). The combined organic layers were dried over Na₂SO₄, filtered, and concentrated under vacuum to give a residue which was purified by flash silica gel chromatography using a gradient of EtOAc/petroleum ether from 0/1 to 1/2. tert-Butyl((5-methyl-6-((1-(naphthalen-1-yl)cyclopropyl)carbamoyl)-1H-indol-2-yl)methyl)carbamate (270 mg, 533 μmol, 77% yield) was obtained as a white solid. M+H⁺=470.1 (LCMS); 1H NMR (400 MHZ, CD₃OD) δ 8.68-8.60 (m, 1H), 7.93-7.86 (m, 2H), 7.83-7.77 (m, 1H), 7.60-7.55 (m, 1H), 7.52-7.47 (m, 1H), 7.46-7.41 (m, 1H), 7.21-7.15 (m, 1H), 7.11-7.03 (m, 1H), 6.17-6.10 (m, 1H), 4.35-4.25 (m, 2H), 2.17-2.12 (m, 3H), 1.47-1.42 (m, 11H), 1.32-1.21 (m, 2H).

### Example 119: 2-(Aminomethyl)-5-methyl-N-(1-(naphthalen-1-yl)cyclopropyl)-1H-indole-6-carboxamide (Compound 170)

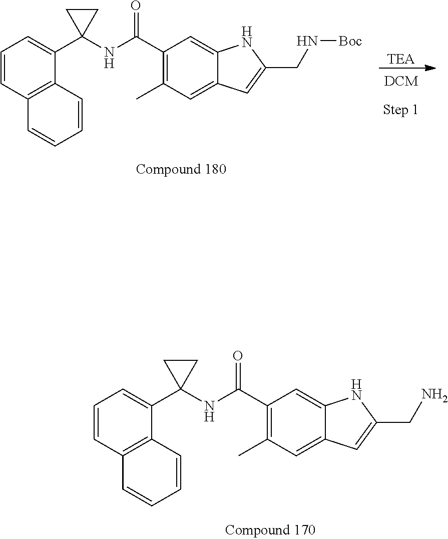

### Step 1: 2-(Aminomethyl)-5-methyl-N-(1-(naphthalen-1-yl)cyclopropyl)-1H-indole-6-carboxamide (Compound 170)

To a solution of tert-butyl((5-methyl-6-((1-(naphthalen-1-yl)cyclopropyl)carbamoyl)-1H-indol-2-yl)methyl)carbamate (200 mg, 426 μmol, 1.0 eq) in DCM (5.0 mL) was added TFA (6.16 g, 54.0 mmol, 4.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Gemini C18 column (150×30 mm, 5 μm); flow rate: 60 mL/min; gradient: 10%-45% B over 8 min; mobile phase A: 0.1% aqueous TFA, mobile phase B: acetonitrile). 2-(Aminomethyl)-5-methyl-N-(1-(naphthalen-1-yl)cyclopropyl)-1H-indole-6-carboxamide (130 mg, 352 μmol, 83% yield, TFA salt) was obtained as a yellow solid. M+H⁺=370.0 (LCMS); ¹H NMR (400 MHZ, CD₃OD) δ 8.67-8.56 (m, 1H), 7.94-7.87 (m, 2H), 7.84-7.78 (m, 1H), 7.61-7.41 (m, 3H), 7.29-7.25 (m, 1H), 7.14-7.10 (m, 1H), 6.47-6.44 (m, 1H), 4.24-4.19 (m, 2H), 2.17-2.13 (m, 3H), 1.50-1.43 (m, 2H), 1.35-1.27 (m, 2H).

### Example 120: 2-(Aminomethyl)-5-methyl-N-(1-(naphthalen-2-yl)cyclopropyl)-1H-indole-6-carboxamide (Compound 248)

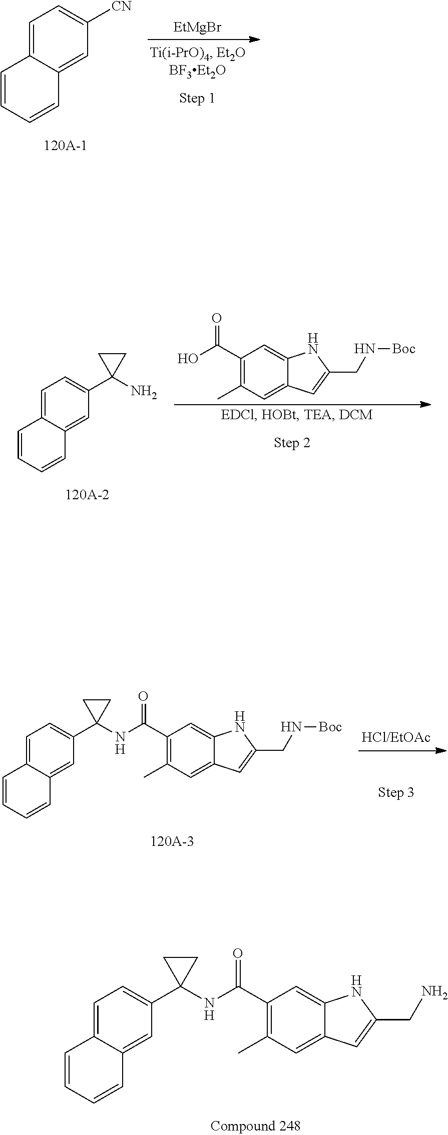

### Step 1: 1-(Naphthalen-2-yl)cyclopropanamine (120A-2)

A mixture of 2-naphthonitrile (2.00 g, 13.1 mmol, 307 μL, 1.0 eq) in anhydrous Et₂O (50 mL) was degassed and purged with N₂ three times. The mixture was stirred at −78° C. To this mixture was added Ti(i-PrO)₄ (4.08 g, 14.4 mmol, 4.24 mL, 1.1 eq) slowly and then EtMgBr (3 M in Et₂O, 9.57 mL, 2.2 eq) was added dropwise to maintain the temperature between-78° C. and −75° C. over 10 min under a N₂ atmosphere. After the addition was complete, the resulting mixture was stirred at the same temperature for 10 min and warmed to 20° C. over 1 h. BF₃·Et₂O (3.71 g, 26.1 mmol, 3.22 mL, 2.0 eq) was added slowly with no obvious temperature change. The mixture was stirred at 20° C. for another 1 h. TLC indicated that the starting material was completely consumed, and a main spot was detected. The reaction mixture was added into a mixture of HCl (1 M aqueous) (30 mL) and MTBE (30 mL) and extracted with MTBE (30 mL×4). The aqueous layer was basified to pH 8 using NaOH (2 M aqueous) and a precipitate was formed. The mixture was filtered through a pad of Celite and the slurry was washed with DCM several times. The combined filtrate was extracted with DCM (50 mL×4). The combined organic layers were dried over Na₂SO₄, filtered, and concentrated under vacuum to give a residue which was purified by flash silica gel chromatography using a gradient of EtOAc/petroleum ether from 0/1 to 1/1. 1-(Naphthalen-2-yl)cyclopropanamine (400 mg, 2.03 mmol, 16% yield) was obtained as a yellow solid. M+H⁺=184.1 (LCMS).

### Step 2: tert-Butyl((5-methyl-6-((1-(naphthalen-2-yl)cyclopropyl)carbamoyl)-1H-indol-2-yl)methyl)carbamate (120A-3)

To a solution of 1-(naphthalen-2-yl)cyclopropanamine (150 mg, 819 μmol, 1.0 eq) and 2-(((tert-butoxycarbonyl)amino)methyl)-5-methyl-1H-indole-6-carboxylic acid (299 mg, 982 μmol, 1.2 eq) in DCM (2.0 mL) were added EDCI (235 mg, 1.23 mmol, 1.5 eq), HOBt (166 mg, 1.23 mmol, 1.5 eq) and TEA (248 mg, 2.46 mmol, 342 μL, 3.0 eq). The mixture was stirred at 25° C. for 16 h. TLC indicated that the starting material was completely consumed. The reaction mixture was poured into H₂O (10 mL) at 25° C. and extracted with EtOAc (10 mL×3). The combined organic layers were washed with brine (10 mL×3), dried over Na₂SO₄, filtered, and concentrated under vacuum to give a residue which was purified by flash silica gel chromatography using a gradient of EtOAc/petroleum ether from 0/1 to 1/1. tert-Butyl((5-methyl-6-((1-(naphthalen-2-yl)cyclopropyl)carbamoyl)-1H-indol-2-yl)methyl)carbamate (150 mg, 256 μmol, 31% yield) was obtained as a yellow solid. M+H⁺=470.3 (LCMS).

### Step 3: 2-(Aminomethyl)-5-methyl-N-(1-(naphthalen-2-yl)cyclopropyl)-1H-indole-6-carboxamide (Compound 248)

To a mixture of tert-butyl((5-methyl-6-((1-(naphthalen-2-yl)cyclopropyl)carbamoyl)-1H-indol-2-yl)methyl)carbamate (100 mg, 213 μmol, 1.0 eq) in EtOAc (1.0 mL) was added HCl/EtOAc (2 M, 10 mL). The mixture was stirred at 25° C. for 1 h. LCMS indicated that the starting material was completely consumed, and the desired mass was detected. The reaction mixture was concentrated under vacuum to give a residue which was purified by preparative HPLC (Phenomenex Luna column (80×30 mm, 3 μm); flow rate: 25 mL/min; gradient: 10%-40% B over 8 min; mobile phase A: 0.04% aqueous HCl, mobile phase B: acetonitrile). 2-(Aminomethyl)-5-methyl-N-(1-(naphthalen-2-yl)cyclopropyl)-1H-indole-6-carboxamide (65.0 mg, 176 μmol, 83% yield, HCl salt) was obtained as a white solid. M+H⁺=370.3 (LCMS); ¹H NMR (400 MHZ, DMSO-d₆) δ 11.37 (s, 1H), 9.03 (s, 1H), 8.53 (br s, 3H), 7.89-7.78 (m, 4H), 7.51-7.37 (m, 5H), 6.49 (s, 1H), 4.20 (s, 2H), 2.40 (s, 3H), 1.37 (br d, J=10.5 Hz, 4H).

### Example 121: 2-(Aminomethyl)-5-methyl-N-(1-phenylcyclopropyl)-1H-indole-6-carboxamide (Compound 246)

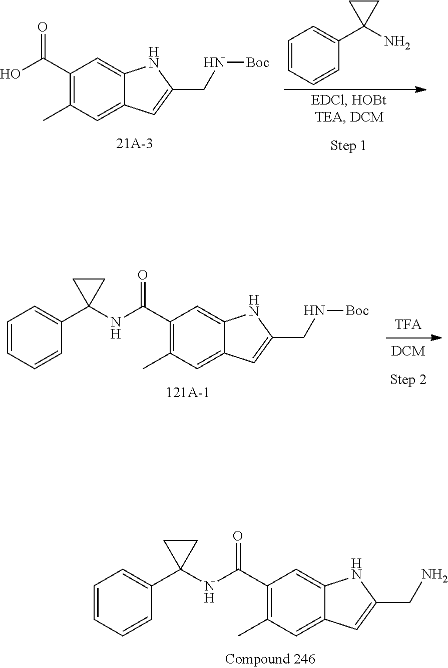

### Step 1: tert-Butyl((5-methyl-6-((1-phenylcyclopropyl)carbamoyl)-1H-indol-2-yl)methyl) carbamate (121A-1)

To a solution of 2-(((tert-butoxycarbonyl)amino)methyl)-5-methyl-1H-indole-6-carboxylic acid (80.0 mg, 263 μmol, 1.0 eq) and 1-phenylcyclopropanamine (35.0 mg, 263 μmol, 1.0 eq) in DCM (5.0 mL) was added TEA (79.8 mg, 789 μmol, 110 μL, 3.0 eq), followed by EDCI (75.6 mg, 394 μmol, 1.5 eq) and HOBt (53.3 mg, 394 μmol, 1.5 eq). The mixture was stirred at 20° C. for 16 h. LCMS indicated that the starting material was completely consumed, and the desired mass was detected. The reaction mixture was poured into H₂O (10 mL) and extracted with EtOAc (8.0 mL×5). The combined organic layers were dried over Na₂SO₄, filtered, and concentrated under vacuum to give a residue which was purified by preparative TLC (EtOAc/petroleum ether=1/2, R_{f}=0.5). tert-Butyl((5-methyl-6-((1-phenylcyclopropyl) carbamoyl)-1H-indol-2-yl)methyl)carbamate (80.0 mg, 191 μmol, 73% yield) was obtained as a yellow oil. M+H⁺=420.2 (LCMS).

### Step 2: 2-(Aminomethyl)-5-methyl-N-(1-phenylcyclopropyl)-1H-indole-6-carboxamide (Compound 246)

To a solution of tert-butyl((5-methyl-6-((1-phenylcyclopropyl)carbamoyl)-1H-indol-2-yl)methyl)carbamate (80.0 mg, 191 μmol, 1.0 eq) in DCM (5.0 mL) was added TFA (6.16 g, 54.0 mmol, 4.0 mL). The mixture was stirred at 20° C. for 1 h. LCMS indicated that the starting material was completely consumed, and the desired mass was detected. The mixture was concentrated under vacuum at 30° C. to give the residue which was purified by preparative HPLC (Phenomenex Gemini C18 column (80×30 mm, 3 μm); flow rate: 25 mL/min; gradient: 30%-60% B over 8 min; mobile phase A: 0.1% aqueous TFA, mobile phase B: acetonitrile). 2-(Aminomethyl)-5-methyl-N-(1-phenylcyclopropyl)-1H-indole-6-carboxamide (9.20 mg, 21.2 μmol, 11% yield, TFA salt) was obtained as a white solid. M+H⁺=320.1 (LCMS); 1H NMR (400 MHZ, DMSO-d₆) δ 11.19-11.04 (m, 1H), 8.91-8.82 (m, 1H), 8.29-8.19 (m, 2H), 7.51-7.42 (m, 1H), 7.40-7.14 (m, 6H), 6.60-6.12 (m, 1H), 4.23-4.13 (m, 2H), 2.39-2.35 (m, 3H), 1.31-1.20 (m, 4H).

### Example 122: N-(1-([1,1′-Biphenyl]-4-yl)cyclopropyl)-2-(aminomethyl)-5-methyl-1H-indole-6-carboxamide (Compound 244)

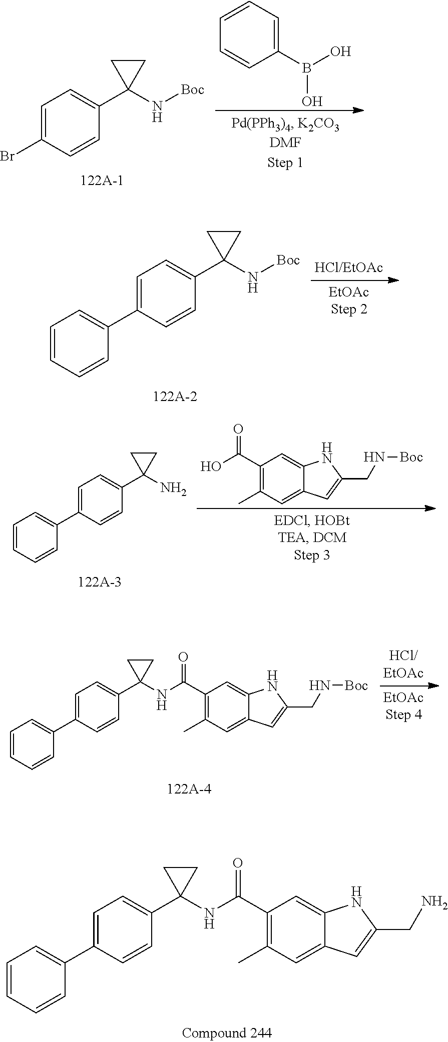

### Step 1: tert-Butyl(1-([1,1′-biphenyl]-4-yl)cyclopropyl)carbamate (122A-2)

A mixture of tert-butyl(1-(4-bromophenyl)cyclopropyl)carbamate (1.00 g, 3.20 mmol, 1.0 eq), phenylboronic acid (781 mg, 6.40 mmol, 2.0 eq) and K₂CO₃ (2 M, 6.41 mL, 4.0 eq) in DMF (30 mL) was degassed and purged with N₂ three times. To the mixture was added Pd(PPh₃)₄ (370 mg, 0.32 mmol, 0.1 eq). The resulting mixture was stirred at 90° C. for 16 h under a N₂ atmosphere. LCMS indicated that the starting material was completely consumed, and the desired mass was detected. The reaction mixture was allowed to cool to room temperature, poured into H₂O (30 mL) and extracted with EtOAc (30 mL×3). The combined organic layers were dried over Na₂SO₄, filtered, and concentrated under vacuum to give a residue which was purified by flash silica gel chromatography using a gradient of EtOAc/petroleum ether from 0/1 to 1/20. tert-Butyl(1-([1,1′-biphenyl]-4-yl)cyclopropyl)carbamate (500 mg, 1.62 mmol, 50% yield) was obtained as a white solid. M−56+H⁺=254.1 (LCMS).

### Step 2: 1-([1,1′-Biphenyl]-4-yl)cyclopropanamine (122A-3)

To a stirred solution of tert-butyl(1-([1,1′-biphenyl]-4-yl)cyclopropyl)carbamate (200 mg, 646 μmol, 1.0 eq) in EtOAc (3.0 mL) was added HCl/EtOAc (4 M, 3.0 mL). The mixture was stirred at 20° C. for 2 h. LCMS indicated that the starting material was completely consumed, and the desired mass was detected. The mixture was concentrated under vacuum at 30° C. to give 1-([1,1′-biphenyl]-4-yl)cyclopropanamine (150 mg, 720 μmol, 88% yield, HCl salt) as a white solid. M+H⁺=210.1 (LCMS); ¹H NMR (400 MHZ, DMSO-d₆) δ 8.98 (br s, 3H), 7.77-7.61 (m, 4H), 7.56-7.43 (m, 4H), 7.41-7.33 (m, 1H), 1.51-1.35 (m, 2H), 1.23 (s, 2H)

### Step 3: tert-Butyl((6-((1-([1,1′-biphenyl]-4-yl)cyclopropyl)carbamoyl)-5-methyl-1H-indol-2-yl)methyl)carbamate (122A-4)

To a solution of 1-([1,1′-biphenyl]-4-yl)cyclopropanamine (80.8 mg, 329 μmol, 1.0 eq) and 2-(((tert-butoxycarbonyl)amino)methyl)-5-methyl-1H-indole-6-carboxylic acid (100 mg, 329 μmol, 1.0 eq) in DCM (3.0 mL) were added TEA (99.8 mg, 987 μmol, 137 μL, 3 eq), EDCI (157 mg, 823 μmol, 2.5 eq) and HOBt (111 mg, 823 μmol, 2.5 eq). The mixture was stirred at 20° C. for 16 h. LCMS indicated that the starting material was completely consumed, and the desired mass was detected. The reaction mixture was poured into H₂O (3.0 mL) and extracted with DCM (3.0 mL×2). The combined organic layers were dried over Na₂SO₄, filtered, and concentrated under vacuum to give a residue which was purified by preparative TLC (EtOAc/petroleum ether=1/1, R_{f}=0.5). tert-Butyl((6-((1-([1,1′-biphenyl]-4-yl)cyclopropyl)carbamoyl)-5-methyl-1H-indol-2-yl)methyl)carbamate (70.0 mg, 141 μmol, 43% yield) was obtained as a white solid. M+H⁺=496.3 (LCMS); ¹H NMR (400 MHZ, DMSO-d₆) δ 10.94 (s, 1H), 8.91 (s, 1H), 7.63 (dd, J=7.9, 19.9 Hz, 4H), 7.50-7.42 (m, 3H), 7.41-7.30 (m, 4H), 7.29-7.24 (m, 1H), 6.41-5.88 (m, 1H), 4.55-4.08 (m, 2H), 2.38 (s, 3H), 1.41 (s, 9H), 1.30 (s, 4H).

### Step 4: N-(1-([1,1′-Biphenyl]-4-yl)cyclopropyl)-2-(aminomethyl)-5-methyl-1H-indole-6-carboxamide (Compound 244)

To a stirred solution of tert-butyl((6-((1-([1,1′-biphenyl]-4-yl)cyclopropyl)carbamoyl)-5-methyl-1H-indol-2-yl)methyl)carbamate (60.0 mg, 121 μmol, 1.0 eq) in EtOAc (3.0 mL) was added HCl/EtOAc (4 M, 2.0 mL). The mixture was stirred at 20° C. for 2 h. LCMS indicated that the starting material was completely consumed, and the desired mass was detected. The mixture was concentrated under vacuum at 30° C. to give a residue which was purified by preparative HPLC (Phenomenex Gemini C18 column (80×30 mm, 3 μm); flow rate: 25 mL/min; gradient: 15%-45% B over 8 min; mobile phase A: 0.04% aqueous HCl, mobile phase B: acetonitrile). N-(1-([1,1′-Biphenyl]-4-yl)cyclopropyl)-2-(aminomethyl)-5-methyl-1H-indole-6-carboxamide (24.8 mg, 57.4 μmol, 47% yield, HCl salt) was obtained as a white solid. M+H⁺=396.1 (LCMS); ¹H NMR (400 MHZ, DMSO-d₆) δ=11.24 (s, 1H), 8.94 (s, 1H), 8.37 (br s, 3H), 7.79-7.56 (m, 4H), 7.54-7.42 (m, 3H), 7.38-7.33 (m, 3H), 6.48 (s, 1H), 4.20 (s, 2H), 2.40 (s, 3H), 1.31 (s, 4H).

### Example 123: 2-(Aminomethyl)-N-(1-(3-bromophenyl)cyclopropyl)-5-methyl-1H-indole-6-carboxamide (Compound 232)

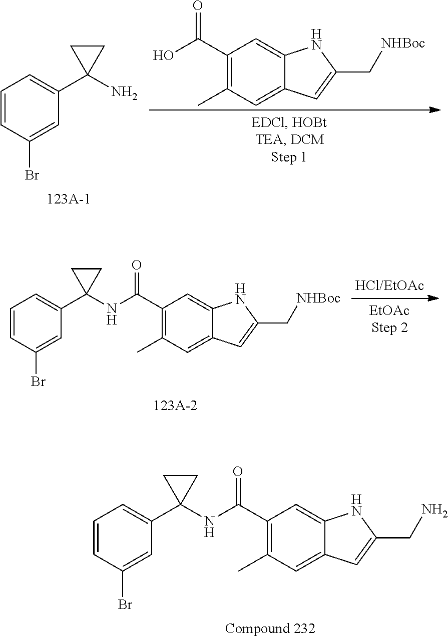 [SMILES: NC1(c2cccc(Br)c2)CC1 ; Cc1cc2cc(CNC(=O)OC(C)(C)C)[nH]c2cc1C(=O)O ; Cc1cc2cc(CNC(=O)OC(C)(C)C)[nH]c2cc1C(=O)NC1(c2cccc(Br)c2)CC1 ; Cc1cc2cc(CN)[nH]c2cc1C(=O)NC1(c2cccc(Br)c2)CC1]

### Step 1: tert-Butyl((6-((1-(3-bromophenyl)cyclopropyl)carbamoyl)-5-methyl-1H-indol-2-yl)methyl)carbamate (123A-2)

To a solution of 1-(3-bromophenyl)cyclopropanamine (209 mg, 990 μmol, 1.0 eq) and 2-(((tert-butoxycarbonyl)amino)methyl)-5-methyl-1H-indole-6-carboxylic acid (300 mg, 990 μmol, 1.0 eq) in DCM (5.0 mL) were added TEA (299 mg, 2.96 mmol, 412 μL, 3.0 eq), EDCI (472 mg, 2.46 mmol, 2.5 eq) and HOBt (333 mg, 2.46 mmol, 2.5 eq). The mixture was stirred at 20° C. for 2 h. LCMS indicated that the starting material was completely consumed, and the desired mass was detected. The reaction mixture was poured into H₂O (5.0 mL) and extracted with DCM (5.0 mL×2). The combined organic layers were dried over Na₂SO₄, filtered, and concentrated under vacuum to give a residue which was purified by flash silica gel chromatography using a gradient of EtOAc/petroleum ether from 0/1 to 1/4. tert-Butyl((6-((1-(3-bromophenyl)cyclopropyl)carbamoyl)-5-methyl-1H-indol-2-yl)methyl)carbamate (200 mg, 401 μmol, 41% yield) was obtained as a yellow solid. M+H⁺=498.1 (LCMS).

### Step 2: 2-(Aminomethyl)-N-(1-(3-bromophenyl)cyclopropyl)-5-methyl-1H-indole-6-carboxamide (Compound 232)

To a stirred solution of tert-butyl((6-((1-(3-bromophenyl)cyclopropyl)carbamoyl)-5-methyl-1H-indol-2-yl)methyl)carbamate (70.0 mg, 140 μmol, 1.0 eq) in EtOAc (3.0 mL) was added HCl/EtOAc (4 M, 3.0 mL). The mixture was stirred at 20° C. for 2 h. LCMS indicated that the starting material was completely consumed, and the desired mass was detected. The mixture was concentrated under vacuum at 30° C. to give a residue which was purified by preparative HPLC (Phenomenex Gemini C18 column (80×30 mm, 3 μm); flow rate: 25 mL/min; gradient: 1%-25% B over 8 min; mobile phase A: 0.04% aqueous HCl, mobile phase B: acetonitrile). 2-(Aminomethyl)-N-(1-(3-bromophenyl)cyclopropyl)-5-methyl-1H-indole-6-carboxamide (20.0 mg, 46.0 μmol, 33% yield, HCl salt) was obtained as a white solid. M+H⁺=398.1 (LCMS); ¹H NMR (400 MHZ, DMSO-d₆) δ 8.95 (s, 1H), 8.56-8.20 (m, 3H), 7.49-7.42 (m, 2H), 7.41-7.35 (m, 2H), 7.28 (t, J=7.9 Hz, 1H), 7.23-7.17 (m, 1H), 6.48 (s, 1H), 4.38-4.03 (m, 2H), 2.38 (s, 3H), 1.30 (s, 4H).

### Example 124: 5-Methyl-2-((methylamino)methyl)-N-(1-(naphthalen-1-yl)cyclopropyl)indoline-6-carboxamide (Compound 221)

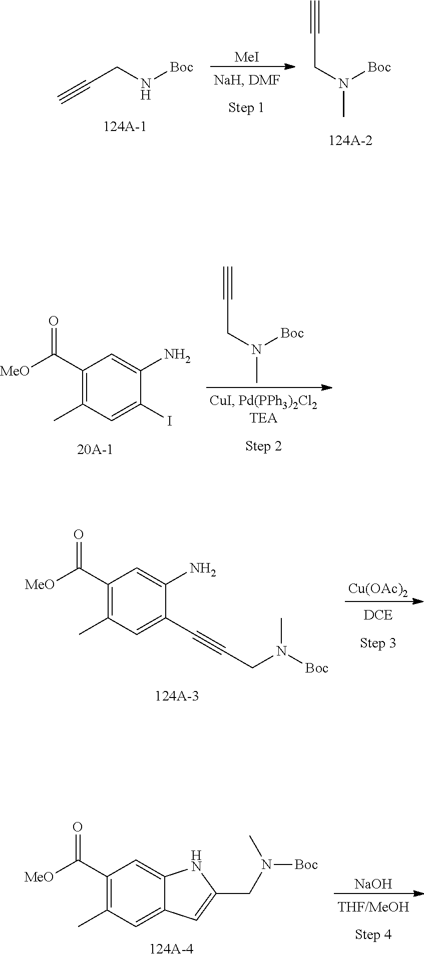

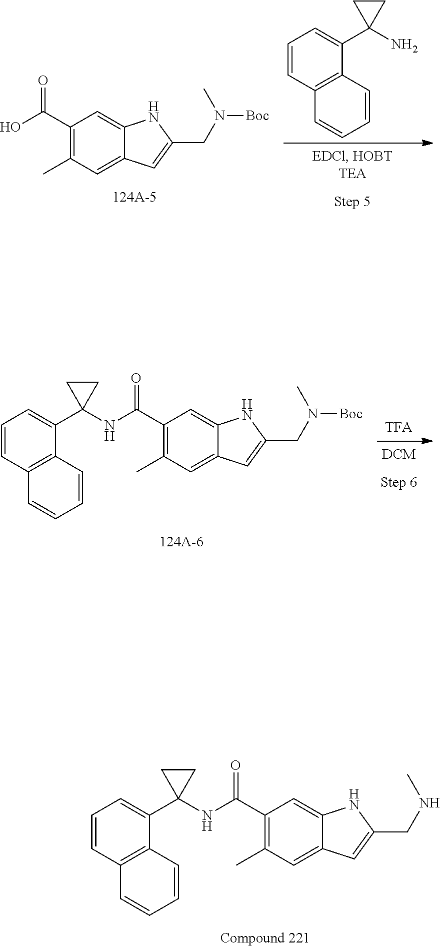 [SMILES: Cc1cc2cc(CN(C)C(=O)OC(C)(C)C)[nH]c2cc1C(=O)O ; NC1(c2cccc3ccccc23)CC1 ; Cc1cc2cc(CN(C)C(=O)OC(C)(C)C)[nH]c2cc1C(=O)NC1(c2cccc3ccccc23)CC1 ; CNCc1cc2cc(C)c(C(=O)NC3(c4cccc5ccccc45)CC3)cc2[nH]1]

### Step 1: tert-Butyl methyl(prop-2-yn-1-yl)carbamate (124A-2)

A mixture of tert-butyl prop-2-yn-1-ylcarbamate (500 mg, 3.22 mmol, 1.0 eq) in THF (15 mL) was degassed and purged with N₂ three times. To the mixture was added sodium hydride (177 mg, 4.43 mmol, 60% purity, 1.5 eq) at 0° C. and the mixture was stirred at the same temperature for 30 min, then Mel (629 mg, 4.43 mmol, 276 μL, 1.5 eq) was added. The mixture was stirred at 25° C. for 16 h. TLC indicated that the starting material was completely consumed. The reaction mixture was poured into saturated aqueous NH₄Cl (20 mL) and extracted with EtOAc (20 mL×4). The combined organic layers were dried over Na₂SO₄, filtered, and concentrated under vacuum to give tert-butyl methyl(prop-2-yn-1-yl)carbamate (430 mg, 2.54 mmol, 79% yield) as a yellow oil, which was used in the next step without any further purification. ¹H NMR (400 MHZ, CDCl₃) δ 4.13-3.97 (m, 2H), 2.94-2.90 (m, 3H), 2.24-2.19 (m, 1H), 1.49-1.46 (m, 9H).

### Step 2: Methyl 5-amino-4-(3-((tert-butoxycarbonyl)(methyl)amino)prop-1-yn-1-yl)-2-methylbenzoate (124A-3)

To a mixture of methyl 5-amino-4-iodo-2-methylbenzoate (700 mg, 2.40 mmol, 1.0 eq), tert-butyl methyl(prop-2-yn-1-yl)carbamate (427 mg, 2.53 mmol, 1.1 eq), CuI (91.6 mg, 481 μmol, 0.2 eq) and Pd(PPh₃)₂Cl₂ (169 mg, 240 μmol, 0.1 eq) was added TEA (8.4 mL). The mixture was stirred at 20° C. for 16 h under a N₂ atmosphere. LCMS indicated that the starting material was completely consumed, and the desired mass was detected. The mixture was poured into H₂O (20 mL) and extracted with DCM (15 mL×8). The combined organic layers were dried over Na₂SO₄, filtered, and concentrated under vacuum to give a residue which was purified by flash silica gel chromatography using a gradient of EtOAc/petroleum ether from 0/1 to 1/2.

Methyl 5-amino-4-(3-((tert-butoxycarbonyl)(methyl)amino)prop-1-yn-1-yl)-2-methylbenzoate (500 mg, 1.50 mmol, 63% yield) was obtained as a yellow solid. M+H⁺=333.2 (LCMS); ¹H NMR (400 MHZ, CDCl₃) δ 7.29-7.27 (m, 1H), 7.15-7.13 (m, 1H), 4.36-4.28 (m, 2H), 3.90-3.85 (m, 3H), 2.99-2.98 (m, 3H), 2.46-2.39 (m, 3H), 1.51-1.47 (m, 9H).

### Step 3: Methyl 2-(((tert-butoxycarbonyl)(methyl)amino)methyl)-5-methyl-1H-indole-6-carboxylate (124A-4)

To a solution of methyl 5-amino-4-(3-((tert-butoxycarbonyl)(methyl)amino)prop-1-yn-1-yl)-2-methylbenzoate (500 mg, 1.50 mmol, 1.0 eq) in DCE (15 mL) was added Cu(OAc)₂ (683 mg, 3.76 mmol, 2.5 eq). The mixture was stirred at 90° C. for 6 h under a N₂ atmosphere. TLC indicated that the starting material was completely consumed. The reaction mixture was allowed to cool to room temperature, poured into H₂O (10 mL) and extracted with DCM (10 mL×5). The combined organic layers were dried over Na₂SO₄, filtered, and concentrated under vacuum to give a residue which was purified by flash silica gel chromatography using a gradient of EtOAc/petroleum ether from 0/1 to 1/2. Methyl 2-(((tert-butoxycarbonyl)(methyl)amino)methyl)-5-methyl-1H-indole-6-carboxylate (500 mg, 1.50 mmol, 100% yield) was obtained as yellow oil. ¹H NMR (400 MHZ, CDCl₃) δ 9.13-8.98 (m, 1H), 8.04-7.98 (m, 1H), 7.41-7.37 (m, 1H), 6.36-6.25 (m, 1H), 4.52-4.35 (m, 2H), 3.96-3.85 (m, 3H), 2.90-2.85 (m, 3H), 2.72-2.62 (m, 3H), 1.51 (br s, 9H).

### Step 4: 2-(((tert-Butoxycarbonyl)(methyl)amino)methyl)-5-methyl-1H-indole-6-carboxylic acid (124A-5)

To a stirred solution of methyl 2-(((tert-butoxycarbonyl)(methyl)amino)methyl)-5-methyl-1H-indole-6-carboxylate (200 mg, 602 μmol, 1.0 eq) in a mixture of MeOH (2.0 mL) and THF (6.0 mL) was added NaOH (2 M aqueous, 4.0 mL, 14 eq). The resulting mixture was stirred at 20° C. for 2 h, then at 70° C. for another 8 h. LCMS indicated that the starting material was completely consumed, and the desired mass was detected. The reaction mixture was allowed to cool to room temperature, poured into H₂O (10 mL) and washed with TBME (5.0 mL×3). The aqueous layer was acidified to pH 5 using HCl (1 M aqueous). The product was extracted with DCM (5.0 mL×8). The combined organic layers were dried over Na₂SO₄, filtered, and concentrated under vacuum to give the crude product 2-(((tert-butoxycarbonyl)(methyl)amino)methyl)-5-methyl-1H-indole-6-carboxylic acid (200 mg) as a red solid, which was used in the next step without any further purification. M+H⁺=319.2 (LCMS); ¹H NMR (400 MHZ, CDCl₃) δ 7.42-7.39 (m, 1H), 6.99-6.98 (m, 1H), 6.34-6.32 (m, 1H), 2.89-2.88 (m, 2H), 2.73-2.70 (m, 3H), 2.29-2.27 (m, 3H), 1.44 (s, 9H).

### Step 5: tert-Butyl methyl((5-methyl-6-((1-(naphthalen-1-yl)cyclopropyl)carbamoyl)-1H-indol-2-yl)methyl)carbamate (124A-6)

To a solution of 2-(((tert-butoxycarbonyl)(methyl)amino)methyl)-5-methyl-1H-indole-6-carboxylic acid (200 mg, 314 μmol, 1.0 eq) in DCM (8.0 mL) were added 1-(naphthalen-1-yl)cyclopropanamine (57.6 mg, 314 μmol, 1.0 eq) and TEA (95.4 mg, 942 μmol, 131 μL, 3.0 eq), followed by EDCI (90.3 mg, 471 μmol, 1.5 eq) and HOBt (63.7 mg, 471 μmol, 1.5 eq). The resulting mixture was stirred at 20° C. for 15 h. LCMS indicated that the starting material was completely consumed, and the desired mass was detected. The mixture was poured into H₂O (10 mL). The product was extracted with DCM (10 mL×4) and the combined organic layers were dried over Na₂SO₄, filtered, and concentrated under vacuum to give a residue which was purified by flash silica gel chromatography using a gradient of EtOAc/petroleum ether from 0/1 to 1/2. tert-Butyl methyl((5-methyl-6-((1-(naphthalen-1-yl)cyclopropyl)carbamoyl)-1H-indol-2-yl)methyl)carbamate (60.0 mg, 124 μmol, 40% yield) was obtained as a yellow oil. M+H⁺=484.3 (LCMS).

### Step 6: 5-Methyl-2-((methylamino)methyl)-N-(1-(naphthalen-1-yl)cyclopropyl)indoline-6-carboxamide (Compound 221)

To a solution of tert-butyl methyl((5-methyl-6-((1-(naphthalen-1-yl)cyclopropyl)carbamoyl)-1H-indol-2-yl)methyl)carbamate (60.0 mg, 124 μmol, 1.0 eq) in EtOAc (4.0 mL) was added HCl/EtOAc (4 M, 3.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Gemini C18 column (80×30 mm, 3 μm); flow rate: 25 mL/min; gradient: 15%-40% B over 8 min; mobile phase A: 0.04% aqueous HCl, mobile phase B: acetonitrile). 5-Methyl-2-((methylamino)methyl)-N-(1-(naphthalen-1-yl)cyclopropyl)indoline-6-carboxamide (10.7 mg, 26.9 μmol, 22% yield) was obtained as a white solid. M+H⁺=384.2 (LCMS); ¹H NMR (400 MHZ, CD₃OD) δ 8.67-8.60 (m, 1H), 7.95-7.88 (m, 2H), 7.84-7.78 (m, 1H), 7.62-7.55 (m, 1H), 7.54-7.41 (m, 2H), 7.32-7.29 (m, 1H), 7.16-7.12 (m, 1H), 6.55-6.50 (m, 1H), 4.32-4.26 (m, 2H), 2.72-2.67 (m, 3H), 2.20-2.13 (m, 3H), 1.50-1.43 (m, 2H), 1.35-1.27 (m, 2H).

### Example 125: 2-((Dimethylamino)methyl)-5-methyl-N-(1-(naphthalen-1-yl)cyclopropyl)-1H-indole-6-carboxamide (Compound 173)

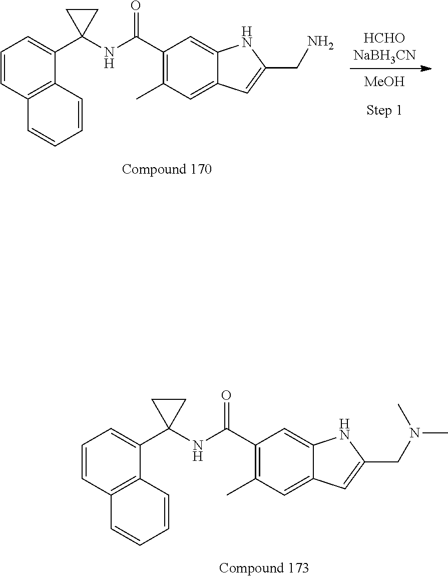 [SMILES: "MeOH" ; Cc1cc2cc(CN)[nH]c2cc1C(=O)NC1(c2cccc3ccccc23)CC1 ; Cc1cc2cc(CN(C)C)[nH]c2cc1C(=O)NC1(c2cccc3ccccc23)CC1]

### Step 1: 2-((Dimethylamino)methyl)-5-methyl-N-(1-(naphthalen-1-yl)cyclopropyl)-1H-indole-6-carboxamide (Compound 173)

To a solution of 2-(aminomethyl)-5-methyl-N-(1-(naphthalen-1-yl)cyclopropyl)-1H-indole-6-carboxamide (90.0 mg, 244 μmol, 1.0 eq) in MeOH (6.0 mL) was added TEA (50.0 μL), followed by the addition of formaldehyde (2.94 g, 36.3 mmol, 2.7 mL, 37% purity in water, 149 eq). The resulting mixture was adjusted to pH 6 with a small amount of AcOH. The mixture was stirred at 20° C. for 30 min, and then NaBH₃CN (123 mg, 1.95 mmol, 8.0 eq) was added. The resulting reaction mixture was stirred at 20° C. for another 14 h. LCMS indicated that the starting material was completely consumed, and the desired mass was detected. The reaction mixture was poured into H₂O (8.0 mL) and extracted with EtOAc (10 mL×4). The combined organic layers were dried over Na₂SO₄, filtered, and concentrated under vacuum to give a residue which was purified by preparative HPLC (Phenomenex Gemini C18 column (80×30 mm, 3 μm); flow rate: 25 mL/min; gradient: 20%-50% B over 8 min; mobile phase A: 0.1% aqueous TFA, mobile phase B: acetonitrile). 2-((Dimethylamino)methyl)-5-methyl-N-(1-(naphthalen-1-yl)cyclopropyl)-1H-indole-6-carboxamide (32.8 mg, 81.1 μmol, 33% yield, TFA salt) was obtained as a white solid. M+H⁺=398.2 (LCMS); ¹H NMR (400 MHZ, DMSO-d₆) δ 11.20-11.16 (m, 1H), 9.10-9.06 (m, 1H), 8.70-8.63 (m, 1H), 7.97-7.90 (m, 1H), 7.86-7.81 (m, 2H), 7.61-7.43 (m, 3H), 7.33-7.29 (m, 1H), 7.14-7.10 (m, 1H), 6.57-6.52 (m, 1H), 4.38-4.33 (m, 2H), 2.73-2.70 (m, 6H), 2.14-2.11 (m, 3H), 1.39-1.34 (m, 2H), 1.20-1.16 (m, 2H).

### Example 126: 2-(1-Aminoethyl)-5-methyl-N-(1-(naphthalen-1-yl)cyclopropyl)-1H-indole-6-carboxamide (Compound 273)

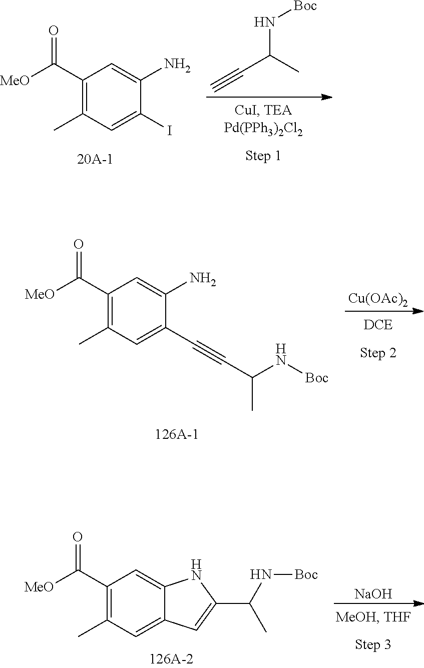 [SMILES: COC(=O)c1cc(N)c(I)cc1C ; C#CC(C)NC(=O)OC(C)(C)C ; COC(=O)c1cc(N)c(C#CC(C)NC(=O)OC(C)(C)C)cc1C ; COC(=O)c1cc2[nH]c(C(C)NC(=O)OC(C)(C)C)cc2cc1C ; "NaOH" ; "Cu(OAc)2"]

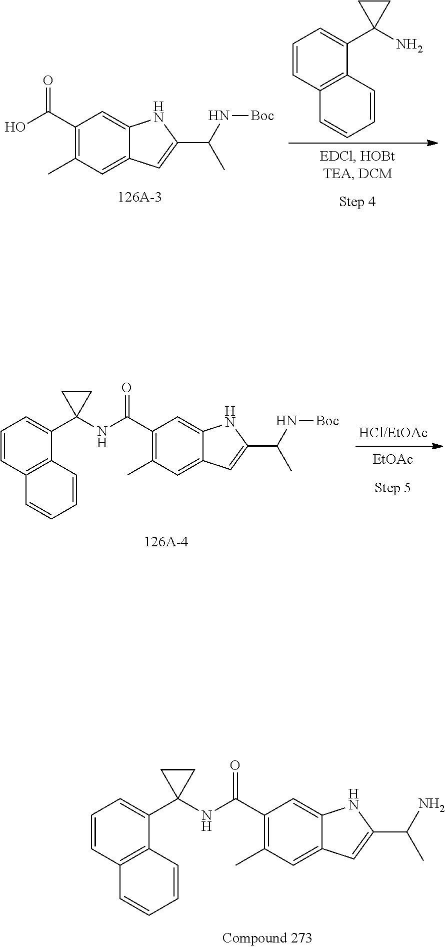 [SMILES: "EtOAc" ; Cc1cc2cc(C(C)NC(=O)OC(C)(C)C)[nH]c2cc1C(=O)O ; NC1(c2cccc3ccccc23)CC1 ; Cc1cc2cc(C(C)NC(=O)OC(C)(C)C)[nH]c2cc1C(=O)NC1(c2cccc3ccccc23)CC1 ; Cc1cc2cc(C(C)N)[nH]c2cc1C(=O)NC1(c2cccc3ccccc23)CC1]

### Step 1: Methyl 5-amino-4-(3-((tert-butoxycarbonyl)amino) but-1-yn-1-yl)-2-methylbenzoate (126A-1)

To a mixture of methyl 5-amino-4-iodo-2-methylbenzoate (400 mg, 1.37 mmol, 1.0 eq), tert-butyl but-3-yn-2-ylcarbamate (244 mg, 1.44 mmol, 90.0 μL, 1.1 eq), CuI (5.23 mg, 27.5 μmol, 0.02 eq) and Pd(PPh₃)₂Cl₂ (19.3 mg, 27.5 μmol, 0.02 eq) was added TEA (3.48 g, 34.4 mmol, 4.78 mL, 25 eq). The mixture was stirred at 20° C. for 16 h under a N₂ atmosphere. TLC indicated that the starting material was completely consumed. The mixture was poured into H₂O (10 mL) and extracted with DCM (10 mL×5). The combined organic layers were dried over Na₂SO₄, filtered, and concentrated under vacuum to give a residue which was purified by flash silica gel chromatography using a gradient of EtOAc/petroleum ether from 0/1 to 1/3. Methyl 5-amino-4-(3-((tert-butoxycarbonyl)amino) but-1-yn-1-yl)-2-methylbenzoate (410 mg, 1.23 mmol, 90% yield) was obtained as a yellow solid. ¹H NMR (400 MHZ, CDCl₃) δ 7.32-7.28 (m, 1H), 7.13 (s, 1H), 4.78-4.68 (m, 1H), 3.88-3.85 (m, 3H), 2.47-2.40 (m, 3H), 1.53-1.50 (m, 3H), 1.49-1.44 (m, 9H).

### Step 2: Methyl 2-(1-((tert-butoxycarbonyl)amino)ethyl)-5-methyl-1H-indole-6-carboxylate (126A-2)

To a solution of methyl 5-amino-4-(3-((tert-butoxycarbonyl)amino) but-1-yn-1-yl)-2-methylbenzoate (410 mg, 1.23 mmol, 1.0 eq) in DCE (30 mL) was added Cu(OAc) 2 (560 mg, 3.09 mmol, 2.5 eq). The mixture was stirred at 130° C. for 6 h under a N₂ atmosphere. TLC indicated that the starting material was completely consumed. The reaction mixture was allowed to cool to room temperature, poured into H₂O (20 mL) and extracted with DCM (20 mL×5). The combined organic layers were dried over Na₂SO₄, filtered, and concentrated under vacuum to give a residue which was purified by flash silica gel chromatography using a gradient of EtOAc/petroleum ether from 0/1 to 1/2. Methyl 2-(1-((tert-butoxycarbonyl)amino)ethyl)-5-methyl-1H-indole-6-carboxylate (320 mg, 963 μmol, 64% yield) was obtained as a yellow solid. ¹H NMR (400 MHZ, CDCl₃) δ 9.13-8.96 (m, 1H), 7.94-7.87 (m, 1H), 7.18-7.10 (m, 1H), 6.19-6.12 (m, 1H), 4.90-4.80 (m, 1H), 4.78-4.68 (m, 1H), 3.81-3.78 (m, 3H), 2.61-2.52 (m, 3H), 1.52 (d, J=6.9 Hz, 3H), 1.40-1.35 (m, 9H).

### Step 3: 2-(1-((tert-Butoxycarbonyl)amino)ethyl)-5-methyl-1H-indole-6-carboxylic acid (126A-3)

To a solution of methyl 2-(1-((tert-butoxycarbonyl)amino)ethyl)-5-methyl-1H-indole-6-carboxylate (120 mg, 361 μmol, 1.0 eq) in a mixture of THF (7.0 mL) and MeOH (1.8 mL) was added NaOH (2 M in aqueous, 1.8 mL, 10 eq). The mixture was stirred at 20° C. for 16 h. Then stirred at 70° C. for another 4 h. LCMS indicated that the starting material was completely consumed, and the desired mass was detected. The reaction mixture was allowed to cool to room temperature, poured into H₂O (10 mL) and washed with MTBE (10 mL×2). The aqueous was acidified to pH 5 with HCl (1 M aqueous). The product was extracted with DCM (15 mL×5). The combined organic layers were dried over Na₂SO₄, filtered, and concentrated under vacuum to give 2-(1-((tert-butoxycarbonyl)amino)ethyl)-5-methyl-1H-indole-6-carboxylic acid (90.0 mg, 283 μmol, 78% yield) as a yellow oil, which was used in the next step without any further purification. M+H⁺=319.2 (LCMS).

### Step 4: tert-Butyl(1-(5-methyl-6-((1-(naphthalen-1-yl)cyclopropyl)carbamoyl)-1H-indol-2-yl)ethyl)carbamate (126A-4)

To a solution of 1-(naphthalen-1-yl)cyclopropanamine (44.0 mg, 240 μmol, 1.0 eq) in DCM (5.0 mL) were added 2-(1-((tert-butoxycarbonyl)amino)ethyl)-5-methyl-1H-indole-6-carboxylic acid (90.0 mg, 240 μmol, 1.0 eq), TEA (72.9 mg, 721 μmol, 100 μL, 3.0 eq), EDCI (69.1 mg, 360 μmol, 1.5 eq) and HOBt (48.7 mg, 360 μmol, 1.5 eq). The mixture was stirred at 20° C. for 16 h. LCMS indicated that the starting material was completely consumed, and the desired mass was detected. The reaction mixture was poured into H₂O (10 mL) and extracted with DCM (10 mL×3). The combined organic layers were dried over Na₂SO₄, filtered, and concentrated under vacuum to give a residue was purified by preparative TLC (EtOAc/petroleum ether=1/2, R_{f}=0.5). tert-Butyl(1-(5-methyl-6-((1-(naphthalen-1-yl)cyclopropyl)carbamoyl)-1H-indol-2-yl)ethyl)carbamate (100 mg, 207 μmol, 86% yield) was obtained as a yellow oil. M+H⁺=484.2 (LCMS).

### Step 5: 2-(1-Aminoethyl)-5-methyl-N-(1-(naphthalen-1-yl)cyclopropyl)-1H-indole-6-carboxamide (Compound 273)

To a solution of tert-butyl(1-(5-methyl-6-((1-(naphthalen-1-yl)cyclopropyl)carbamoyl)-1H-indol-2-yl)ethyl)carbamate (80.0 mg, 165.43 μmol, 1.0 eq) in EtOAc (4.0 mL) was added HCl/EtOAc (4 M, 4.0 mL). The mixture was stirred at 20° C. for 1 h. LCMS indicated that the starting material was completely consumed, and the desired mass was detected. The mixture was concentrated under vacuum to give a residue which was purified by preparative HPLC (Phenomenex Gemini C18 column (80×30 mm, 3 μm); flow rate: 25 mL/min; gradient: 15%-45% B over 8 min; mobile phase A: 0.04% aqueous HCl, mobile phase B: acetonitrile). 2-(1-Aminoethyl)-5-methyl-N-(1-(naphthalen-1-yl)cyclopropyl)-1H-indole-6-carboxamide (25.0 mg, 59.5 μmol, 36% yield, HCl salt) was obtained as a yellow solid. M+H⁺=384.1 (LCMS); ¹H NMR (400 MHZ, DMSO-d₆) δ 11.32-11.24 (m, 1H), 9.08-9.01 (m, 1H), 8.76-8.68 (m, 1H), 8.63-8.47 (m, 3H), 7.98-7.90 (m, 1H), 7.87-7.78 (m, 2H), 7.62-7.44 (m, 3H), 7.30-7.23 (m, 1H), 7.14-7.07 (m, 1H), 6.44-6.35 (m, 1H), 4.60-4.49 (m, 1H), 2.19-2.13 (m, 3H), 1.61-1.54 (m, 3H), 1.40-1.35 (m, 2H), 1.22-1.13 (m, 2H).

### Example 127: 5-Methyl-2-(morpholin-3-yl)-N-(1-(naphthalen-1-yl)cyclopropyl)-1H-indole-6-carboxamide (Compound 260)

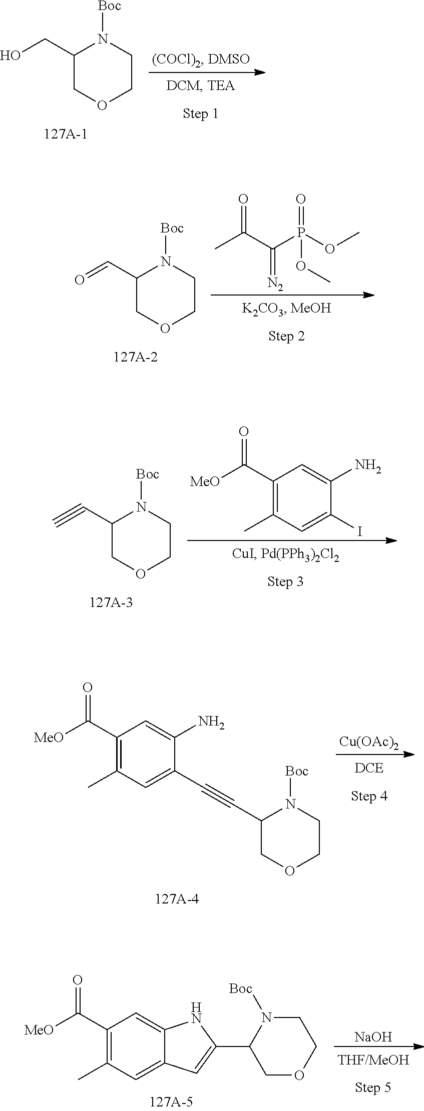

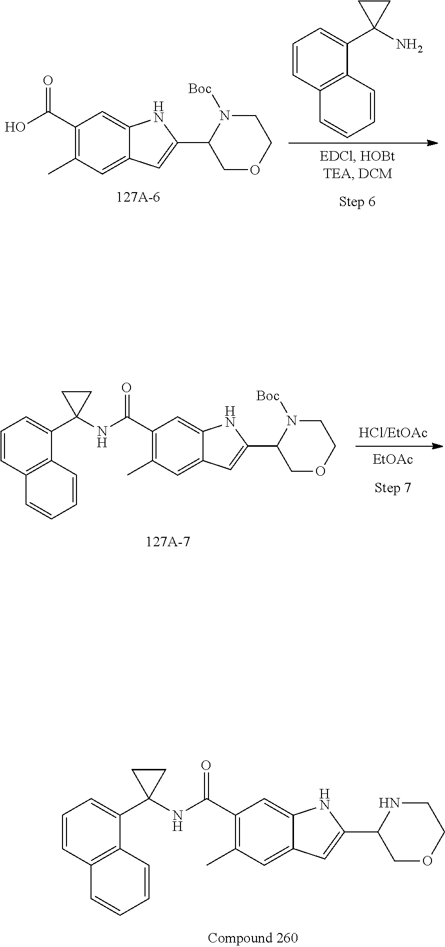 [SMILES: "EtOAc" ; Cc1cc2cc(C3COCCN3C(=O)OC(C)(C)C)[nH]c2cc1C(=O)O ; NC1(c2cccc3ccccc23)CC1 ; Cc1cc2cc(C3COCCN3C(=O)OC(C)(C)C)[nH]c2cc1C(=O)NC1(c2cccc3ccccc23)CC1 ; Cc1cc2cc(C3COCCN3)[nH]c2cc1C(=O)NC1(c2cccc3ccccc23)CC1]

### Step 1: tert-Butyl 3-formylmorpholine-4-carboxylate (127A-2)

To a stirred solution of (COCl)₂ (643 mg, 5.06 mmol, 0.40 mL, 1.1 eq) in DCM (18 mL) was added DMSO (863 mg, 11.1 mmol, 0.80 mL, 2.4 eq) at −78° C. After 15 min, a solution of tert-butyl 3-formylmorpholine-4-carboxylate (1.00 g, 4.60 mmol, 1.0 eq) in DCM (10 mL) was added to the mixture dropwise. The reaction mixture was stirred at −78° C. for another 2 h. TEA (2.33 g, 23.0 mmol, 3.20 mL, 5.0 eq) was added and the reaction mixture was stirred at −78° C. for 30 min then was warmed to 20° C. and stirred for another 1 h. TLC indicated that the starting material was completely consumed. The reaction mixture was poured into H₂O (30 mL) and extracted with DCM (15 mL×2). The combined organic layers were dried over Na₂SO₄, filtered, and concentrated under vacuum to give the crude product tert-butyl 3-formylmorpholine-4-carboxylate (1.00 g, 70% purity) as a colorless oil, which was used in the next step without any further purification.

### Step 2: tert-Butyl 3-ethynylmorpholine-4-carboxylate (127A-3)

To a solution of tert-butyl 3-formylmorpholine-4-carboxylate (1.00 g, 4.65 mmol, 1.0 eq) and dimethyl(1-diazo-2-oxopropyl)phosphonate (1.34 g, 6.97 mmol, 1.5 eq) in MeOH (30 mL) was added K₂CO₃ (2.57 g, 18.6 mmol, 4.0 eq). The mixture was stirred at 20° C. for 16 h. TLC indicated that the starting material was completely consumed. The reaction mixture was poured into H₂O (30 mL) and extracted with DCM (15 mL×2). The combined organic layers were dried over Na₂SO₄, filtered, and concentrated under vacuum to give the crude product tert-butyl 3-ethynylmorpholine-4-carboxylate (800 mg, 85% purity) as a colorless oil, which was used in the next step without any further purification.

### Step 3: tert-Butyl 3-((2-amino-4-(methoxycarbonyl)-5-methylphenyl)ethynyl)morpholine-4-carboxylate (127A-4)

A mixture of methyl 5-amino-4-iodo-2-methylbenzoate (317 mg, 1.09 mmol, 1.0 eq), tert-butyl 3-ethynylmorpholine-4-carboxylate (230 mg, 1.09 mmol, 1.0 eq), Pd(PPh₃)₂Cl₂ (76.4 mg, 109 μmol, 0.1 eq) and CuI (10.4 mg, 54.4 μmol, 0.05 eq) in TEA (5.0 mL) was degassed and purged with N₂ three times. The mixture was stirred at 70° C. for 3 h under a N₂ atmosphere. LCMS indicated that starting material was completely consumed, and the desired mass was detected. The reaction mixture was allowed to cool to room temperature, poured into H₂O (5.0 mL) and extracted with DCM (3.0 mL×2). The combined organic layers were dried over Na₂SO₄, filtered, and concentrated under vacuum to give a residue which was purified by flash silica gel chromatography using a gradient of EtOAc/petroleum ether from 1/100 to 1/10. tert-Butyl 3-((2-amino-4-(methoxycarbonyl)-5-methylphenyl)ethynyl) morpholine-4-carboxylate (400 mg, 1.07 mmol, 49% yield) was obtained as a brown oil. M−56+H⁺=319.1 (LCMS).

### Step 4: tert-Butyl 3-(5-methyl-6-((1-(naphthalen-1-yl)cyclopropyl)carbamoyl)-1H-indol-2-yl) morpholine-4-carboxylate (127A-5)

To a solution of tert-butyl 3-((2-amino-4-(methoxycarbonyl)-5-methylphenyl)ethynyl)morpholine-4-carboxylate (100 mg, 267 μmol, 1.0 eq) in DCE (10 mL) was added Cu(OAc) 2 (97.0 mg, 534 μmol, 2.0 eq) under a N₂ atmosphere. The mixture was stirred at 100° C. for 1 h in a microwave (400 W). TLC indicated that the starting material was completely consumed. The reaction mixture was allowed to cool to room temperature, poured into H₂O (5.0 mL) and extracted with EtOAc (3.0 mL×2). The combined organic layers were dried over Na₂SO₄, filtered, and concentrated under vacuum to give a residue which was purified by preparative TLC (EtOAc/petroleum ether=2/1, R_{f}=0.7). tert-Butyl 3-(5-methyl-6-((1-(naphthalen-1-yl)cyclopropyl)carbamoyl)-1H-indol-2-yl) morpholine-4-carboxylate (80.0 mg, 214 μmol, 40% yield) was obtained as a white solid.

### Step 5: 2-(4-(tert-Butoxycarbonyl) morpholin-3-yl)-5-methyl-1H-indole-6-carboxylic acid (127A-6)

To a solution of tert-butyl 3-(5-methyl-6-((1-(naphthalen-1-yl)cyclopropyl)carbamoyl)-1H-indol-2-yl) morpholine-4-carboxylate (70.0 mg, 187 μmol, 1.0 eq) in a mixture of THF (4.0 mL) and MeOH (1.0 mL) was added NaOH (2 M aqueous, 500 μL, 5.0 eq). The mixture was stirred at 80° C. for 2 h. LCMS indicated that starting material was completely consumed, and the desired mass was detected. The reaction mixture was allowed to cool to room temperature, poured into H₂O (5.0 mL) and washed with MTBE (2.0 mL×2). The aqueous layer was acidified to pH 5 with HCl (1 M aqueous). The product was extracted with EtOAc (2.0 mL×3). The combined organic layers were dried over Na₂SO₄, filtered, and concentrated under vacuum to give the crude product 2-(4-(tert-butoxycarbonyl) morpholin-3-yl)-5-methyl-1H-indole-6-carboxylic acid (70.0 mg, 80% purity) as a white solid, which was used in the next step without any further purification. M+H⁺=361.1 (LCMS)

### Step 6: tert-Butyl 3-(5-methyl-6-((1-(naphthalen-1-yl)cyclopropyl)carbamoyl)-1H-indol-2-yl) morpholine-4-carboxylate (127A-7)

To a solution of 2-(4-(tert-butoxycarbonyl) morpholin-3-yl)-5-methyl-1H-indole-6-carboxylic acid (70.0 mg, 194 μmol, 1.0 eq) and 1-(naphthalen-1-yl)cyclopropanamine (28.5 mg, 155 μmol, 0.8 eq) in DCM (5.0 mL) were added TEA (39.3 mg, 388 μmol, 54.4 μL, 2.0 eq), EDCI (44.7 mg, 233 μmol, 1.2 eq) and HOBt (31.5 mg, 233 μmol, 1.2 eq). The mixture was stirred at 20° C. for 18 h. LCMS indicated that starting material was completely consumed, and the desired mass was detected. The reaction mixture was poured into H₂O (5.0 mL) and extracted with DCM (3.0 mL×2). The combined organic layers were dried over Na₂SO₄, filtered, and concentrated under vacuum to give a residue which was purified by preparative TLC (EtOAc/petroleum ether=1/1, R_{f}=0.6). tert-Butyl 3-(5-methyl-6-((1-(naphthalen-1-yl)cyclopropyl)carbamoyl)-1H-indol-2-yl) morpholine-4-carboxylate (70.0 mg, 133 μmol, 69% yield) was obtained as a white solid. M+H⁺=526.2 (LCMS)

### Step 7: 5-Methyl-2-(morpholin-3-yl)-N-(1-(naphthalen-1-yl)cyclopropyl)-1H-indole-6-carboxamide (Compound 260)

To a stirred solution of tert-butyl 3-(5-methyl-6-((1-(naphthalen-1-yl)cyclopropyl)carbamoyl)-1H-indol-2-yl) morpholine-4-carboxylate (50.0 mg, 95.1 μmol, 1.0 eq) in EtOAc (5.0 mL) was added HCl/EtOAc (4 M, 1.0 mL). The mixture was stirred at 20° C. for 2 h. LCMS indicated that starting material was completely consumed, and the desired mass was detected. The mixture was concentrated under vacuum to give a residue which was purified by preparative HPLC (Phenomenex Gemini C18 column (80×30 mm, 3 μm); flow rate: 25 mL/min; gradient: 5%-50% B over 8 min; mobile phase A: 0.04% aqueous HCl, mobile phase B: acetonitrile). 5-Methyl-2-(morpholin-3-yl)-N-(1-(naphthalen-1-yl)cyclopropyl)-1H-indole-6-carboxamide (16.9 mg, 35.7 μmol, 38% yield, HCl salt) was obtained as a white solid. M+H⁺=426.1 (LCMS); ¹H NMR (400 MHZ, DMSO-d₆) δ 11.29 (br s, 1H), 9.84 (br s, 2H), 9.16-8.96 (m, 1H), 8.78-8.63 (m, 1H), 7.98-7.92 (m, 1H), 7.86-7.80 (m, 2H), 7.62-7.44 (m, 3H), 7.32-7.28 (m, 1H), 7.15-7.10 (m, 1H), 6.58-6.50 (m, 1H), 4.73-4.50 (m, 1H), 4.17-3.94 (m, 2H), 3.91-3.75 (m, 2H), 3.31-3.19 (m, 2H), 2.16 (s, 3H), 1.43-1.32 (m, 2H), 1.23-1.13 (m, 2H).

### Example 128: 2-(2-Aminoethyl)-5-methyl-N-(1-(naphthalen-1-yl)cyclopropyl)-1H-indole-6-carboxamide (Compound 202)

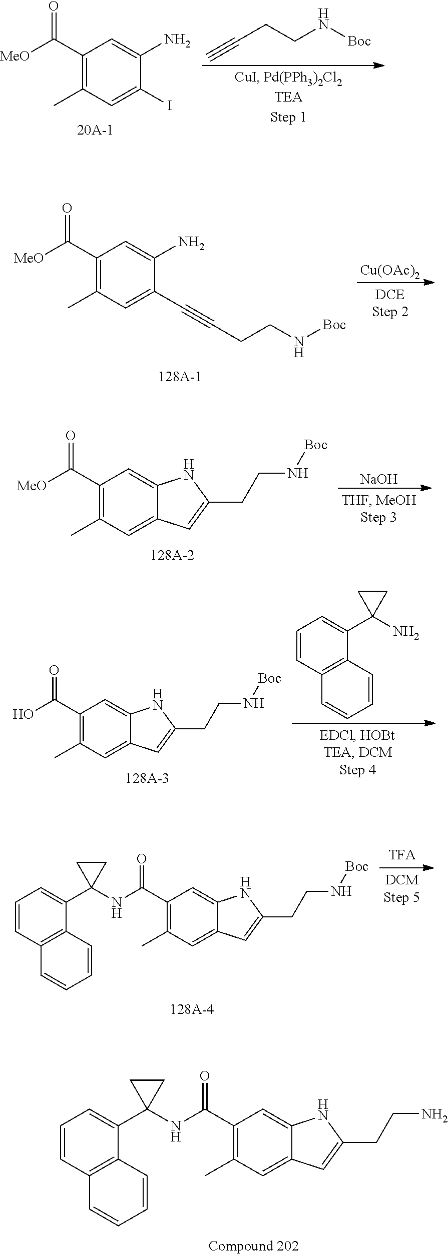

### Step 1: Methyl 5-amino-4-(4-((tert-butoxycarbonyl)amino) but-1-yn-1-yl)-2-methylbenzoate (128A-1)

To a mixture of methyl 5-amino-4-iodo-2-methylbenzoate (500 mg, 1.72 mmol, 1.0 eq), tert-butyl but-3-yn-1-ylcarbamate (349 mg, 2.06 mmol, 1.2 eq), CuI (65.4 mg, 344 μmol, 0.2 eq) and Pd(PPh₃)₂Cl₂ (121 mg, 172 μmol, 0.1 eq) was added TEA (4.35 g, 42.9 mmol, 6.0 mL, 25 eq). The mixture was stirred at 20° C. for 16 h under a N₂ atmosphere. LCMS indicated that the starting material was completely consumed, and the desired mass was detected. The mixture was poured into H₂O (10 mL) and extracted with DCM (10 mL×5). The combined organic layers were dried over Na₂SO₄, filtered, and concentrated under vacuum to give a residue which was purified by flash silica gel chromatography using a gradient of EtOAc/petroleum ether from 0/1 to 1/1. Methyl 5-amino-4-(3-((tert-butoxycarbonyl)amino) but-1-yn-1-yl)-2-methyl benzoate (420 mg, 1.26 mmol, 74% yield) was obtained as a yellow oil. M+H⁺=333.2 (LCMS).

### Step 2: Methyl 2-(2-((tert-butoxycarbonyl)amino)ethyl)-5-methyl-1H-indole-6-carboxylate (128A-2)

To a solution of methyl 5-amino-4-(3-((tert-butoxycarbonyl)amino) but-1-yn-1-yl)-2-methyl benzoate (300 mg, 903 μmol, 1.0 eq) in DCE (15 mL) was added Cu(OAc)₂ (410 mg, 2.26 mmol, 2.5 eq). The mixture was stirred at 90° C. for 6 h under a N₂ atmosphere. TLC indicated that the starting material was completely consumed. The reaction mixture was allowed to cool to room temperature, poured into H₂O (20 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/2. Methyl 2-(2-((tert-butoxycarbonyl)amino)ethyl)-5-methyl-1H-indole-6-carboxylate (210 mg, 632 μmol, 70% yield) was obtained as a yellow oil. ¹H NMR (400 MHZ, CDCl₃) δ 8.84-8.58 (m, 1H), 8.02-7.96 (m, 1H), 7.35 (s, 1H), 6.27-6.15 (m, 1H), 3.91-3.88 (m, 3H), 3.55-3.43 (m, 2H), 3.05-2.92 (m, 2H), 2.69-2.63 (m, 3H), 1.46-1.42 (m, 9H).

### Step 3: 2-(2-((tert-Butoxycarbonyl)amino)ethyl)-5-methyl-1H-indole-6-carboxylic acid (128A-3)

To a solution of methyl 2-(2-((tert-butoxycarbonyl)amino)ethyl)-5-methyl-1H-indole-6-carboxylate (200 mg, 602 μmol, 1.0 eq) in a mixture of THF (6.0 mL) and MeOH (2.0 mL) was added NaOH (2 M in aqueous, 4.2 mL, 14 eq). The mixture was stirred at 20° C. for 2 h, then at 70° C. for another 6 h. LCMS indicated that the starting material was completely consumed, and the desired mass was detected. The reaction mixture was allowed to cool to room temperature, poured into H₂O (10 mL) and washed with MTBE (10 mL×2). The aqueous was acidified to pH 5 with HCl (1 M aqueous). The product was extracted with DCM (5.0 mL×3). The combined organic layers were dried over Na₂SO₄, filtered, and concentrated under vacuum to give crude product 2-(2-((tert-butoxycarbonyl)amino)ethyl)-5-methyl-1H-indole-6-carboxylic acid (210 mg, 90% purity) as a yellow solid, which was used in the next step without any further purification. M−H⁻=317.2 (LCMS).

### Step 4: tert-Butyl(2-(5-methyl-6-((1-(naphthalen-1-yl)cyclopropyl)carbamoyl)-1H-indol-2-yl)ethyl)carbamate (128A-4)

To a solution of 1-(naphthalen-1-yl)cyclopropanamine (104 mg, 569 μmol, 1.0 eq) and 2-(2-((tert-butoxycarbonyl)amino)ethyl)-5-methyl-1H-indole-6-carboxylic acid (201 mg, 569 μmol, 90% purity, 1.0 eq) in DCM (5.0 mL) were added TEA (173 mg, 1.71 mmol, 237 μL, 3.0 eq), EDCI (164 mg, 853 μmol, 1.5 eq) and HOBt (115 mg, 853 μmol, 1.5 eq). The mixture was stirred at 20° C. for 16 h. LCMS indicated that the starting material was completely consumed, and the desired mass was detected. The reaction mixture was poured into H₂O (10 mL) and extracted with DCM (3.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/2. tert-Butyl(2-(5-methyl-6-((1-(naphthalen-1-yl)cyclopropyl)carbamoyl)-1H-indol-2-yl)ethyl)carbamate (170 mg, 352 μmol, 62% yield) was obtained as a yellow oil. M+H⁺=484.3 (LCMS).

### Step 5: 2-(2-Aminoethyl)-5-methyl-N-(1-(naphthalen-1-yl)cyclopropyl)-1H-indole-6-carboxamide (Compound 202)

To a solution of tert-butyl(2-(5-methyl-6-((1-(naphthalen-1-yl)cyclopropyl)carbamoyl)-1H-indol-2-yl)ethyl)carbamate (170 mg, 352 μmol, 1.0 eq) in DCM (10 mL) was added TFA (10.0 g, 87.8 mmol, 6.50 mL, 250 eq). The mixture was stirred at 20° C. for 1 h. LCMS indicated that the starting material was completely consumed, and the desired mass was detected. The mixture was concentrated under vacuum to give the residue which was purified by preparative HPLC (Phenomenex Gemini C18 column (150×30 mm, 5 μm); flow rate: 60 mL/min; gradient: 10%-45% B over 8 min; mobile phase A: 0.1% aqueous TFA, mobile phase B: acetonitrile). 2-(2-Aminoethyl)-5-methyl-N-(1-(naphthalen-1-yl)cyclopropyl)-1H-indole-6-carboxamide (120 mg, 313 μmol, 89% yield, TFA salt) was obtained as a white solid. M+H⁺=384.1 (LCMS); ¹H NMR (400 MHZ, CD₃OD) δ 8.66-8.61 (m, 1H), 7.94-7.88 (m, 2H), 7.83-7.78 (m, 1H), 7.61-7.55 (m, 1H), 7.53-7.41 (m, 2H), 7.22-7.19 (m, 1H), 7.07-7.05 (m, 1H), 6.22-6.19 (m, 1H), 3.28-3.23 (m, 2H), 3.11-3.05 (m, 2H), 2.18-2.14 (m, 3H), 1.48-1.43 (m, 2H), 1.34-1.28 (m, 2H).

### Example 129: 5-Methyl-2-(2-(methylamino)ethyl)-N-(1-(naphthalen-1-yl)cyclopropyl)-1H-indole-6-carboxamide (Compound 222)

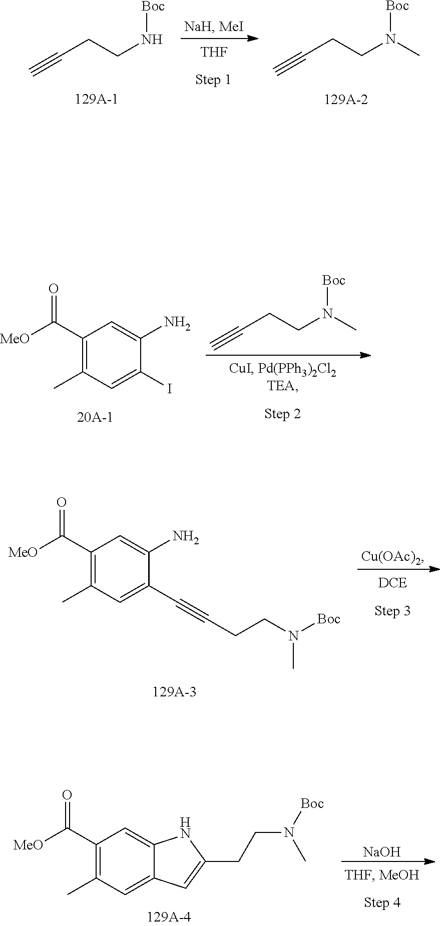

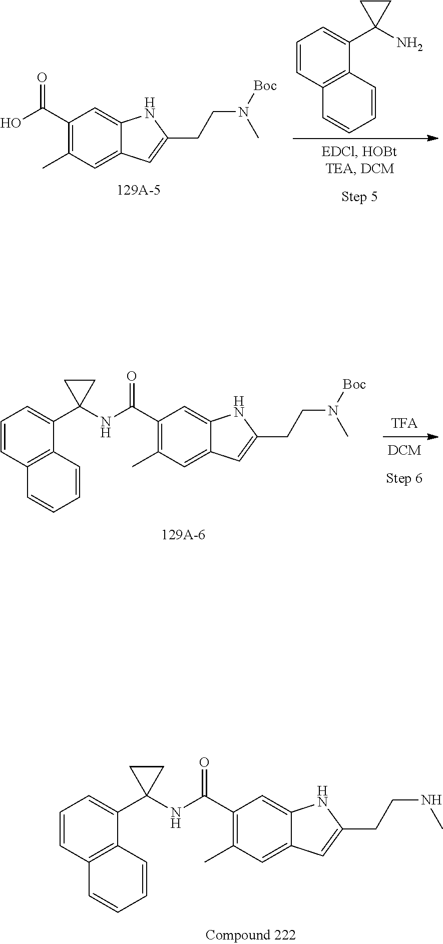

### Step 1: tert-Butyl but-3-yn-1-yl(methyl)carbamate (129A-2)

To a mixture of tert-butyl but-3-yn-1-ylcarbamate (500 mg, 2.95 mmol, 1.0 eq) in THF (15 mL) was added sodium hydride (177 mg, 4.43 mmol, 60% purity, 1.5 eq) at 0° C. The mixture was stirred at 0° C. for 30 min under a N₂ atmosphere, and Mel (629 mg, 4.43 mmol, 276 μL, 1.5 eq) was added. The resulting mixture was stirred at 25° C. for 4 h. TLC indicated that the starting material was completely consumed. The reaction mixture was quenched with saturated aqueous NH₄Cl (10 mL) and extracted with EtOAc (10 mL×4). The combined organic layers were dried over Na₂SO₄, filtered, and concentrated under vacuum to give tert-butyl but-3-yn-1-yl(methyl)carbamate (520 mg, 2.84 mmol, 96% yield) as a yellow oil, which was used in the next step without any further purification. ¹H NMR (400 MHZ, CDCl₃) δ 3.45-3.36 (m, 2H), 3.35-3.25 (m, 1H), 2.94-2.91 (m, 3H), 2.45-2.38 (m, 2H), 1.47 (s, 9H).

### Step 2: Methyl 5-amino-4-(4-((tert-butoxycarbonyl)(methyl)amino)but-1-yn-1-yl)-2-methylbenzoate (129A-3)

To a mixture of methyl 5-amino-4-iodo-2-methylbenzoate (700 mg, 2.40 mmol, 1.0 eq), tert-butyl but-3-yn-1-yl(methyl)carbamate (507 mg, 2.77 mmol, 1.2 eq), Pd(PPh₃)₂Cl₂ (169 mg, 240 μmol, 0.1 eq) and CuI (91.6 mg, 481 μmol, 0.2 eq) was added TEA (6.08 g, 60.1 mmol, 8.40 mL, 25 eq). The mixture was stirred at 20° C. for 16 h under a N₂ atmosphere. LCMS indicated that the starting material was completely consumed, and the desired mass was detected. The mixture was poured into H₂O (20 mL) and extracted with DCM (15 mL×4). The combined organic layers were dried over Na₂SO₄, filtered, and concentrated under vacuum to give a residue which was purified by flash silica gel chromatography using a gradient of EtOAc/petroleum ether from 0/1 1/2. to Methyl 5-amino-4-(4-((tert-butoxycarbonyl)(methyl)amino)but-1-yn-1-yl)-2-methylbenzoate (570 mg, 1.65 mmol, 68% yield) was obtained as a yellow oil. M+H⁺=347.2 (LCMS); ¹H NMR (400 MHZ, CDCl₃) δ 7.27-7.24 (m, 1H), 7.12-7.09 (m, 1H), 3.89-3.84 (m, 3H), 3.56-3.44 (m, 2H), 3.01-2.91 (m, 3H), 2.75-2.66 (m, 2H), 2.46-2.39 (m, 3H), 1.50-1.44 (m, 9H).

### Step 3: Methyl 2-(2-((tert-butoxycarbonyl)(methyl)amino)ethyl)-5-methyl-1H-indole-6-carboxylate (129A-4)

To a solution of methyl 5-amino-4-(4-((tert-butoxycarbonyl)(methyl)amino)but-1-yn-1-yl)-2-methylbenzoate (570 mg, 1.65 mmol, 1.0 eq) in DCE (20 mL) was added Cu(OAc) 2 (747 mg, 4.11 mmol, 2.5 eq). The mixture was stirred at 90° C. for 6 h under a N₂ atmosphere. TLC indicated that the starting material was completely consumed. The reaction mixture was allowed to cool to room temperature, poured into H₂O (10 mL) and extracted with DCM (10 mL×5). The combined organic layers were dried over Na₂SO₄, filtered, and concentrated under vacuum to give a residue which was purified by flash silica gel chromatography using a gradient of EtOAc/petroleum ether from 0/1 to 1/1. Methyl 2-(2-((tert-butoxycarbonyl)(methyl)amino)ethyl)-5-methyl-1H-indole-6-carboxylate (440 mg, 1.27 mmol, 77% yield) was obtained as a yellow solid. ¹H NMR (400 MHZ, CDCl₃) δ 8.05-7.95 (m, 1H), 7.36-7.32 (m, 1H), 7.13-7.07 (m, 1H), 6.23-6.18 (m, 1H), 3.88-3.85 (m, 3H), 3.14-3.01 (m, 2H), 2.89-2.79 (m, 3H), 2.73-2.69 (m, 2H), 2.44-2.41 (m, 3H), 1.47 (s, 9H).

### Step 4: 2-(2-((tert-Butoxycarbonyl)(methyl)amino)ethyl)-5-methyl-1H-indole-6-carboxylic acid (129A-5)

To a solution of methyl 2-(2-((tert-butoxycarbonyl)(methyl)amino)ethyl)-5-methyl-1H-indole-6-carboxylate (440 mg, 1.27 mmol, 1.0 eq) in a mixture of MeOH (3.0 mL) and THF (9.0 mL) was added NaOH (2 M aqueous, 8.9 mL, 14 eq). The resulting mixture was stirred 20° C. for 2 h, then at 70° C. for another 8 h. LCMS indicated that the starting material was completely consumed, and the desired mass was detected. The reaction mixture was allowed to cool to room temperature, poured into H₂O (10 mL) and washed with MTBE (5.0 mL×3). The aqueous was acidified to pH 5 with HCl (1 M aqueous) and extracted with DCM (10 mL×8), the combined organic layers were dried over Na₂SO₄, filtered, and concentrated under vacuum to give 2-(2-((tert-butoxycarbonyl)(methyl)amino)ethyl)-5-methyl-1H-indole-6-carboxylic acid (410 mg, 1.23 mmol, 97% yield) as a red solid, which was used in the next step without any further purification. M−H⁻=331.2 (LCMS); ¹H NMR (400 MHZ, CDCl₃) δ 8.21-8.16 (m, 1H), 7.43-7.39 (m, 1H), 6.24-6.21 (m, 1H), 3.56-3.47 (m, 2H), 3.12-3.00 (m, 2H), 2.73-2.70 (m, 3H), 2.49-2.46 (m, 3H), 1.46-1.42 (m, 9H).

### Step 5: tert-Butyl methyl(2-(5-methyl-6-((1-(naphthalen-1-yl)cyclopropyl)carbamoyl)-1H-indol-2-yl)ethyl)carbamate (129A-6)

To a solution of 2-(2-((tert-butoxycarbonyl)(methyl)amino)ethyl)-5-methyl-1H-indole-6-carboxylic acid (100 mg, 301 μmol, 1.0 eq) in DCM (8.0 mL) were added 1-(naphthalen-1-yl)cyclopropanamine (55.1 mg, 301 μmol, 1.0 eq), TEA (91.3 mg, 903 μmol, 126 μL, 3.0 eq), EDCI (86.5 mg, 451 μmol, 1.5 eq) and HOBt (61.0 mg, 451 μmol, 1.5 eq). The mixture was stirred at 20° C. for 16 h. LCMS indicated that the starting material was completely consumed, and the desired mass was detected. The mixture was poured into H₂O (10 mL) and extracted with DCM (10 mL×4). The combined organic layers were dried over Na₂SO₄, filtered, and concentrated under vacuum to give a residue was purified by preparative TLC (EtOAc/petroleum ether=1/2, R_{f}=0.4). tert-Butyl methyl(2-(5-methyl-6-((1-(naphthalen-1-yl)cyclopropyl)carbamoyl)-1H-indol-2-yl)ethyl)carbamate (90.0 mg, 181 μmol, 60% yield) was obtained as a yellow oil. M+H⁺=498.2 (LCMS).

### Step 6: 5-Methyl-2-(2-(methylamino)ethyl)-N-(1-(naphthalen-1-yl)cyclopropyl)-1H-indole-6-carboxamide (Compound 222)

To a solution of tert-butyl methyl(2-(5-methyl-6-((1-(naphthalen-1-yl)cyclopropyl) carbamoyl)-1H-indol-2-yl)ethyl)carbamate (90.0 mg, 181 μmol, 1.0 eq) in DCM (5.0 mL) was added TFA (3.08 g, 27.0 mmol, 2.00 mL, 149 eq). The mixture was stirred at 20° C. for 1 h. LCMS indicated that the starting material was completely consumed, and the desired mass was detected. The mixture was concentrated under vacuum to give the residue which was purified by preparative HPLC (Phenomenex Gemini C18 column (80×30 mm, 3 μm); flow rate: 25 mL/min; gradient: 15%-40% B over 8 min; mobile phase A: 0.1% aqueous TFA, mobile phase B: acetonitrile). 5-Methyl-2-(2-(methylamino)ethyl)-N-(1-(naphthalen-1-yl)cyclopropyl)-1H-indole-6-carboxamide (18.6 mg, 46.8 μmol, 26% yield, TFA salt) was obtained as a white solid. M+H⁺=398.1 (LCMS); ¹H NMR (400 MHZ, CD₃OD) δ 9.22-9.10 (m, 1H), 8.60-8.54 (m, 1H), 7.92-7.86 (m, 2H), 7.84-7.77 (m, 1H), 7.60-7.53 (m, 1H), 7.52-7.40 (m, 2H), 7.01-6.97 (m, 1H), 6.52-6.47 (m, 1H), 3.27-3.19 (m, 2H), 2.96-2.86 (m, 2H), 2.77-2.72 (m, 3H), 1.92-1.87 (m, 3H), 1.46-1.38 (m, 2H), 1.33-1.25 (m, 2H).

### Example 130: 5-Methyl-2-(morpholinomethyl)-N-(1-(naphthalen-1-yl)cyclopropyl)-1H-indole-6-carboxamide (Compound 213)

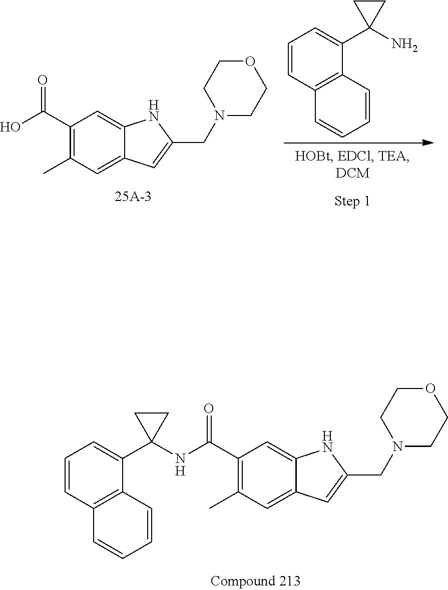

### Step 1: 5-Methyl-2-(morpholinomethyl)-N-(1-(naphthalen-1-yl)cyclopropyl)-1H-indole-6-carboxamide (Compound 213)

To a solution of 1-(naphthalen-1-yl)cyclopropanamine (90.2 mg, 492 μmol, 1.0 eq) and 5-methyl-2-(morpholinomethyl)-1H-indole-6-carboxylic acid (135 mg, 492 μmol, 1.0 eq) in DCM (10 mL) were added TEA (149 mg, 1.48 mmol, 206 μL, 3.0 eq), EDCI (142 mg, 738 μmol, 1.5 eq) and HOBt (99.8 mg, 738 μmol, 1.5 eq). The mixture was stirred at 25° C. for 16 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 55%-75% B over 8 min; mobile phase A: 0.2% aqueous FA, mobile phase B: acetonitrile). 5-Methyl-2-(morpholinomethyl)-N-(1-(naphthalen-1-yl)cyclopropyl)-1H-indole-6-carboxamide (24.6 mg, 55.7 μmol, 11% yield, FA salt) was obtained as a white solid. M+H⁺=440.2 (LCMS); ¹H NMR (400 MHZ, CDCl₃) δ 9.09-8.90 (m, 1H), 8.48 (d, J=8.3 Hz, 1H), 7.97 (d, J=7.0 Hz, 1H), 7.91 (d, J=7.7 Hz, 1H), 7.81 (d, J=8.4 Hz, 1H), 7.62-7.45 (m, 3H), 7.26 (s, 1H), 7.18 (s, 1H), 6.54 (s, 1H), 6.27 (s, 1H), 3.86-3.67 (m, 6H), 2.56 (br s, 4H), 2.29 (s, 3H), 1.61-1.55 (m, 2H), 1.45-1.36 (m, 2H).

### Example 131: 2-((Benzylamino)methyl)-5-methyl-N-(1-(naphthalen-1-yl)cyclopropyl)-1H-indole-6-carboxamide (Compound 229)

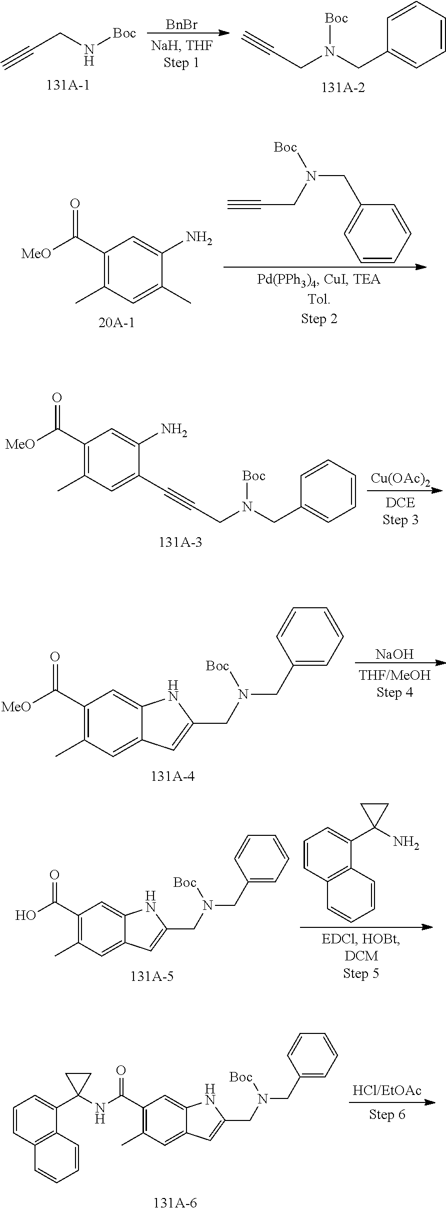

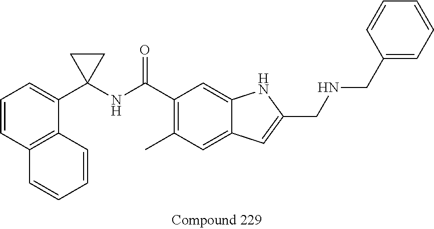 [SMILES: Cc1cc2cc(CNCc3ccccc3)[nH]c2cc1C(=O)NC1(c2cccc3ccccc23)CC1]

### Step 1: tert-Butyl benzyl(prop-2-yn-1-yl)carbamate (131A-2)

A solution of tert-butyl prop-2-yn-1-ylcarbamate (500 mg, 3.22 mmol, 1.0 eq) in THF (10 mL) was degassed and purged with N₂ three times. To the mixture was added sodium hydride (193 mg, 4.83 mmol, 60% purity, 1.5 eq) in portions at 0° C. The mixture was stirred at the same temperature for 30 min. Then (bromomethyl)benzene (827 mg, 4.83 mmol, 574 μL, 1.5 eq) was added and the resulting mixture was stirred at 25° C. for 16 h under a N₂ atmosphere. LCMS indicated that the starting material was completely consumed, and the desired mass was detected. The reaction mixture was poured into ice water (15 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. tert-Butyl benzyl(prop-2-yn-1-yl)carbamate (260 mg, 1.06 mmol, 33% yield) was obtained as a white solid. M−56+H⁺=190.1 (LCMS); ¹H NMR (400 MHZ, CDCl₃) δ 7.37-7.31 (m, 2H), 7.28 (br dd, J=4.0, 6.3 Hz, 3H), 4.57 (s, 2H), 4.20-3.80 (m, 2H), 2.22 (br s, 1H), 1.58-1.43 (m, 9H).

### Step 2: Methyl 5-amino-4-(3-(benzyl(tert-butoxycarbonyl)amino)prop-1-yn-1-yl)-2-methylbenzoate (131A-3)

To a solution of methyl 5-amino-4-iodo-2-methylbenzoate (150 mg, 515 μmol, 1.0 eq) and tert-butyl benzyl(prop-2-yn-1-yl)carbamate (164 mg, 670 μmol, 1.3 eq) in THF (2.0 mL) were added Pd(PPh₃)₄ (11.9 mg, 10.3 μmol, 0.02 eq), copper (I) iodide (1.96 mg, 10.3 μmol, 0.02 eq) and TEA (130 mg, 1.29 mmol, 179 μL, 2.5 eq) at 20° C. The resulting mixture was stirred at 25° C. for 16 h under a N₂ atmosphere. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/5. Methyl 5-amino-4-(3-(benzyl(tert-butoxycarbonyl)amino)prop-1-yn-1-yl)-2-methylbenzoate (150 mg, 367 μmol, 71% yield) was obtained as a colorless oil. M+H⁺=409.3 (LCMS); ¹H NMR (400 MHZ, CDCl₃) δ 7.63-7.39 (m, 7H), 4.80 (s, 2H), 4.57-4.36 (m, 2H), 4.06 (s, 3H), 2.62 (s, 3H), 1.69 (br s, 9H).

### Step 3: Methyl 2-((benzyl(tert-butoxycarbonyl)amino)methyl)-5-methyl-1H-indole-6-carboxylate (131A-4)

A solution of methyl 5-amino-4-(3-(benzyl(tert-butoxycarbonyl)amino)prop-1-yn-1-yl)-2-methylbenzoate (150 mg, 367 μmol, 1.0 eq) in THF (30 mL) was degassed and purged with N₂ three times. To the mixture was added Cu(OAc) 2 (167 mg, 918 μmol, 2.5 eq) at 20° C. The resulting mixture was stirred at 130° C. for 5 h under a N₂ atmosphere. TLC indicated that the starting material was completely consumed. The reaction mixture was allowed to cool to room temperature, poured into H₂O (10 mL) and extracted with DCM (3.0 mL×3). The combined organic layers were dried over Na₂SO₄, filtered, and concentrated under vacuum to give a residue which was purified by preparative TLC (EtOAc/petroleum ether=1/3, R_{f}=0.6). Methyl 2-((benzyl(tert-butoxycarbonyl)amino)methyl)-5-methyl-1H-indole-6-carboxylate (90.0 mg, 220 μmol, 60% yield) was obtained as a colorless oil. ¹H NMR (400 MHZ, CDCl₃) δ 9.18-9.02 (m, 1H), 8.02 (br s, 1H), 7.38 (s, 1H), 7.35 (s, 1H), 7.33 (s, 1H), 7.31 (br d, J=6.7 Hz, 1H), 7.22 (br d, J=5.5 Hz, 2H), 6.24 (s, 1H), 4.40 (s, 4H), 3.91 (s, 3H), 2.68 (s, 3H), 1.51 (br s, 9H).

### Step 4: 2-((Benzyl(tert-butoxycarbonyl)amino)methyl)-5-methyl-1H-indole-6-carboxylic acid (131A-5)

To a solution of methyl 2-((benzyl(tert-butoxycarbonyl)amino)methyl)-5-methyl-1H-indole-6-carboxylate (80.0 mg, 196 μmol, 1.0 eq) in a mixture of MeOH (3.0 mL) and THF (9.0 mL) was added NaOH (2 M aqueous, 3.0 mL, 31 eq). The mixture was stirred at 70° C. for 2 h. LCMS indicated that the starting material was completely consumed, and the desired mass was detected. The reaction mixture was allowed to cool to room temperature, poured into H₂O (15 mL) and extracted with MTBE (5.0 mL×3). The aqueous layer was acidified to pH 5 using HCl (1 M aqueous) and extracted with EtOAc (8.0 mL×3). The combined organic layers were dried over Na₂SO₄, filtered, and concentrated under vacuum to give the crude product 2-((benzyl(tert-butoxycarbonyl)amino)methyl)-5-methyl-1H-indole-6-carboxylic acid (90.0 mg), which was used in the next step without any further purification. M+H⁺=395.2 (LCMS).

### Step 5: tert-Butyl benzyl((5-methyl-6-((1-(naphthalen-1-yl)cyclopropyl)carbamoyl)-1H-indol-2-yl)methyl)carbamate (131A-6)

To a solution of 1-(naphthalen-1-yl)cyclopropanamine (40.0 mg, 218 μmol, 1.0 eq) and 2-((benzyl(tert-butoxycarbonyl)amino)methyl)-5-methyl-1H-indole-6-carboxylic acid (86.1 mg, 218 μmol, 1.0 eq) in DCM (2.0 mL) were added TEA (66.3 mg, 655 μmol, 91.2 μL, 3.0 eq), EDCI (83.7 mg, 437 μmol, 2.0 eq) and HOBt (59.0 mg, 437 μmol, 2.0 eq). The mixture was stirred at 25° C. for 2 h. LCMS indicated that the starting material was completely consumed, and the desired mass was detected. The reaction mixture was poured into H₂O (5.0 mL) and extracted with DCM (3.0 mL×3). The combined organic layers were dried over Na₂SO₄, filtered, and concentrated under vacuum to give a residue which was purified by preparative TLC (EtOAc/petroleum ether=1/5, R_{f}=0.4). tert-Butyl benzyl((5-methyl-6-((1-(naphthalen-1-yl)cyclopropyl)carbamoyl)-1H-indol-2-yl)methyl)carbamate (70.0 mg, 125 μmol, 57% yield) was obtained as a colorless oil. M+H⁺=560.4 (LCMS); ¹H NMR (400 MHZ, CDCl₃) δ 8.90 (br s, 1H), 8.48 (d, J=8.3 Hz, 1H), 7.98 (d, J=7.0 Hz, 1H), 7.91 (d, J=8.3 Hz, 1H), 7.81 (d, J=8.1 Hz, 1H), 7.63-7.56 (m, 1H), 7.55-7.46 (m, 2H), 7.36-7.28 (m, 3H), 7.25 (s, 1H), 7.22-7.13 (m, 2H), 6.54 (s, 1H), 6.16 (s, 1H), 4.40-4.32 (m, 4H), 2.29 (s, 3H), 1.48 (br s, 9H), 1.43-1.38 (m, 2H), 1.27 (t, J=7.2 Hz, 2H).

### Step 6: 2-((Benzylamino)methyl)-5-methyl-N-(1-(naphthalen-1-yl)cyclopropyl)-1H-indole-6-carboxamide (Compound 229)

To a stirred solution of tert-butyl benzyl((5-methyl-6-((1-(naphthalen-1-yl)cyclopropyl) carbamoyl)-1H-indol-2-yl)methyl)carbamate (50.0 mg, 89.3 μmol, 1.0 eq) in EtOAc (1.0 mL) was added HCl/EtOAc (4 M, 8.0 mL). The mixture was stirred at 20° C. for 2 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 10%-60% B over 8 min; mobile phase A: 0.04% aqueous HCl, mobile phase B: acetonitrile). 2-((Benzylamino)methyl)-5-methyl-N-(1-(naphthalen-1-yl)cyclopropyl)-1H-indole-6-carboxamide (4.80 mg, 9.14 μmol, 10% yield, HCl salt) was obtained as a white solid. M+H⁺=460.1 (LCMS); ¹H NMR (400 MHZ, CDCl₃) δ 9.99-9.68 (m, 3H), 8.58 (br d, J=7.5 Hz, 1H), 7.91 (br dd, J=7.2, 14.7 Hz, 2H), 7.81 (br d, J=8.6 Hz, 1H), 7.55-7.38 (m, 5H), 7.34 (br s, 3H), 6.89 (br s, 1H), 6.70-6.53 (m, 1H), 6.05 (br d, J=1.8 Hz, 1H), 3.97-3.75 (m, 4H), 2.07 (s, 3H), 1.56 (br d, J=3.4 Hz, 2H), 1.39 (br s, 2H).

### Example 132: 5-Methyl-6-((1-(naphthalen-1-yl)cyclopropyl)carbamoyl)-1H-indole-2-carboxylic acid (Compound 204)

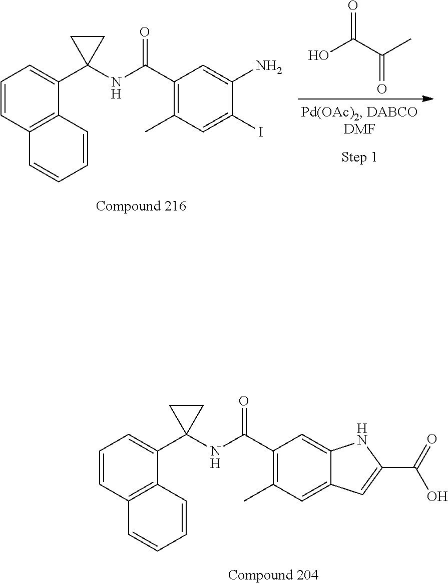

### Step 1: 5-Methyl-6-((1-(naphthalen-1-yl)cyclopropyl)carbamoyl)-1H-indole-2-carboxylic acid (Compound 204)

To a solution of 5-amino-4-iodo-2-methyl-N-(1-(naphthalen-1-yl)cyclopropyl)benzamide (100 mg, 226 μmol, 1.0 eq) and 2-oxopropanoic acid (99.6 mg, 1.13 mmol, 79.6 μL, 5.0 eq) in DMF (8.0 mL) was added 1,4-diazabicyclo[2.2.2]octane (76.1 mg, 678 μmol, 74.6 μL, 3.0 eq). The mixture was stirred at 20° C. for 30 min. Then the mixture was added Pd(OAc) 2 (2.54 mg, 11.3 μmol, 0.05 eq) and stirred at 105° C. for 16 h. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were washed with brine (5.0 mL×2) then dried over Na₂SO₄, filtered, and concentrated under vacuum to give a residue which was purified by preparative HPLC (Phenomenex Gemini C18 column (75×30 mm, 3 μm); flow rate: 25 mL/min; gradient: 30%-60% B over 8 min; mobile phase A: 0.2% aqueous FA, mobile phase B: acetonitrile). 5-Methyl-6-((1-(naphthalen-1-yl)cyclopropyl)carbamoyl)-1H-indole-2-carboxylic acid (9.73 mg, 25.3 μmol, 11% yield, FA salt) was obtained as a yellow solid. M+H⁺=385.1 (LCMS); ¹H NMR (400 MHZ, DMSO-d₆) δ 11.72-11.61 (m, 1H), 9.16-9.09 (m, 1H), 8.71-8.65 (m, 1H), 7.98-7.91 (m, 1H), 7.87-7.81 (m, 2H), 7.62-7.51 (m, 3H), 7.50-7.44 (m, 1H), 7.37-7.34 (m, 1H), 7.13-7.10 (m, 1H), 2.13-2.09 (m, 3H), 1.40-1.34 (m, 2H), 1.21-1.16 (m, 2H).

### Example 133: 5-Methyl-N⁶-(1-(naphthalen-1-yl)cyclopropyl)-1H-indole-2,6-dicarboxamide (Compound 205)

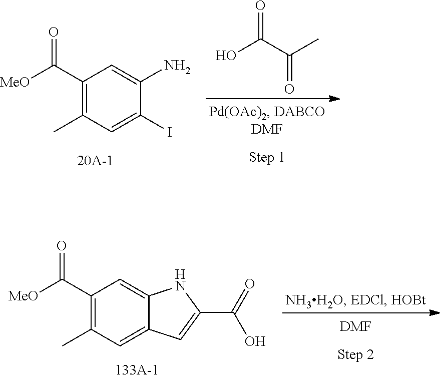

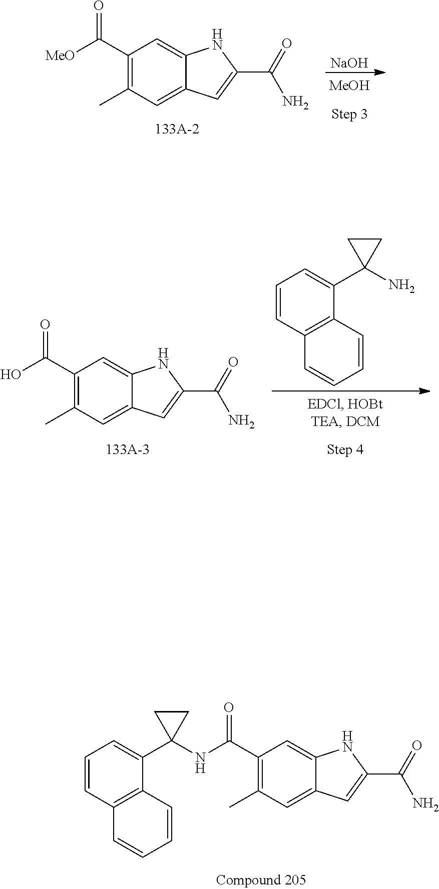 [SMILES: "MeOH" ; COC(=O)c1cc2[nH]c(C(N)=O)cc2cc1C ; "NaOH" ; Cc1cc2cc(C(N)=O)[nH]c2cc1C(=O)O ; NC1(c2cccc3ccccc23)CC1 ; Cc1cc2cc(C(N)=O)[nH]c2cc1C(=O)NC1(c2cccc3ccccc23)CC1]

### Step 1: 6-(Methoxycarbonyl)-5-methyl-1H-indole-2-carboxylic acid (133A-1)

To a solution of methyl 5-amino-4-iodo-2-methylbenzoate (500 mg, 1.72 mmol, 1.0 eq) and 2-oxopropanoic acid (756 mg, 8.59 mmol, 605 L, 5.0 eq) in DMF (15 mL) was added 1,4-diazabicyclo[2.2.2]octane (578 mg, 5.15 mmol, 567 μL, 3.0 eq). The reaction mixture was stirred at 20° C. for 30 min, then Pd(OAc) 2 (19.3 mg, 85.9 μmol, 0.05 eq) was added. The mixture was stirred at 105° C. for 16 h. LCMS indicated that the starting material was completely consumed, and the desired mass was detected. The reaction mixture was poured into H₂O (20 mL) and extracted with EtOAc (10 mL×3). The combined organic layers were washed with brine (10×3 mL), were dried over Na₂SO₄, filtered, and concentrated under vacuum to give crude product 6-(methoxycarbonyl)-5-methyl-1H-indole-2-carboxylic acid (420 mg, 92% purity) as a yellow oil, which was used in the next step without any further purification. M+H⁺=233.9 (LCMS); ¹H NMR (400 MHz, DMSO-d₆) δ 12.04-11.92 (m, 1H), 8.03-7.99 (m, 1H), 7.97-7.93 (m, 1H), 7.57-7.51 (m, 1H), 7.06-6.99 (m, 1H), 3.85-3.82 (m, 3H), 2.57-2.54 (m, 3H).

### Step 2: Methyl 2-carbamoyl-5-methyl-1H-indole-6-carboxylate (133A-2)

To a solution of 6-(methoxycarbonyl)-5-methyl-1H-indole-2-carboxylic acid (200 mg, 858 μmol, 1.0 eq) and HOBt (151 mg, 1.11 mmol, 1.3 eq) in DMF (10 mL) was added EDCI (214 mg, 1.11 mmol, 1.3 eq). The reaction mixture was stirred at 20° C. for 0.6 h, then NH₃·H₂O (5.46 g, 39.0 mmol, 6.0 mL, 25% purity in water, 46 eq) was added. The mixture was stirred at 25° C. for 16 h. LCMS indicated that the starting material was completely consumed, and the desired mass was detected. The reaction mixture was poured into H₂O (20 mL) and extracted with EtOAc (10 mL×4). The combined organic layers were washed with brine (20×2 mL) then the combined organic layers were dried over Na₂SO₄, filtered, and concentrated under vacuum to give crude product methyl 2-carbamoyl-5-methyl-1H-indole-6-carboxylate (270 mg, 70% purity) as a yellow oil, which was used in the next step without any further purification. M+H⁺=233.1 (LCMS); ¹H NMR (400 MHZ, DMSO-d₆) δ 11.80-11.75 (m, 1H), 8.08-8.04 (m, 1H), 8.01-7.98 (m, 1H), 7.96-7.94 (m, 1H), 7.52-7.49 (m, 1H), 6.89-6.84 (m, 1H), 3.84-3.80 (m, 3H), 2.57-2.55 (m, 3H).

### Step 3: 2-Carbamoyl-5-methyl-1H-indole-6-carboxylic acid (133A-3)

To a solution of methyl 2-carbamoyl-5-methyl-1H-indole-6-carboxylate (200 mg, 603 μmol, 70% purity, 1.0 eq) in MeOH (10 mL) was added NaOH (1 M, 3.1 mL, 5.0 eq). The mixture was stirred at 20° C. for 16 h. LCMS indicated that the starting material was completely consumed, and the desired mass was detected. The mixture was poured into H₂O (10 mL) and washed with MTBE (10 mL×2). The aqueous layer was acidified to pH 5 using HCl (1 M aqueous). The product was extracted with DCM (10 mL×5). The combined organic layers were dried over Na₂SO₄, filtered, and concentrated under vacuum to give crude product 2-carbamoyl-5-methyl-1H-indole-6-carboxylic acid (150 mg, 80% purity) as a yellow solid, which was used in the next step without any further purification. M−H⁻=217.1 (LCMS); ¹H NMR (400 MHZ, DMSO-d₆) δ 11.76-11.67 (m, 1H), 8.08-7.97 (m, 2H), 7.50-7.42 (m, 2H), 7.10-7.05 (m, 1H), 6.91-6.84 (m, 1H), 2.57-2.55 (m, 3H).

### Step 4: 5-Methyl-N⁶-(1-(naphthalen-1-yl)cyclopropyl)-1H-indole-2,6-dicarboxamide (Compound 205)

To a solution of 1-(naphthalen-1-yl)cyclopropanamine (67.2 mg, 367 μmol, 1.0 eq) and 2-carbamoyl-5-methyl-1H-indole-6-carboxylic acid (100 mg, 367 μmol, 1.0 eq) were added TEA (111 mg, 1.10 mmol, 153 μL, 3.0 eq), EDCI (105 mg, 550 μmol, 1.5 eq) and HOBt (74.3 mg, 550 μmol, 1.5 eq). The mixture was stirred at 20° C. for 16 h. LCMS indicated that the starting material was completely consumed, and the desired mass was detected. The reaction mixture was poured into H₂O (10 mL) and extracted with DCM (10 mL×3). The combined organic layers were dried over Na₂SO₄, filtered, and concentrated under vacuum to give a residue which was purified by preparative HPLC (Phenomenex Gemini C18 column (70×30 mm, 3 μm); flow rate: 25 mL/min; gradient: 35%-65% B over 8 min; mobile phase A: 0.2% aqueous FA, mobile phase B: acetonitrile). 5-Methyl-N⁶-(1-(naphthalen-1-yl)cyclopropyl)-1H-indole-2,6-dicarboxamide (29.0 mg, 75.6 μmol, 21% yield, FA salt) was obtained as a white solid. M+H⁺=384.1 (LCMS); ¹H NMR (400 MHz, DMSO-d₆) δ 11.50-11.43 (m, 1H), 9.12-9.05 (m, 1H), 8.71-8.64 (m, 1H), 7.98-7.88 (m, 2H), 7.87-7.79 (m, 2H), 7.62-7.43 (m, 3H), 7.35-7.27 (m, 2H), 7.14-7.08 (m, 1H), 7.01-6.95 (m, 1H), 2.12-2.08 (m, 3H), 1.36 (br s, 2H), 1.20-1.12 (m, 2H).

### Example 134: N₂-Methoxy-N_{2,5}-dimethyl-N⁶-(1-(naphthalen-1-yl)cyclopropyl)-1H-indole-2,6-dicarboxamide (Compound 225)

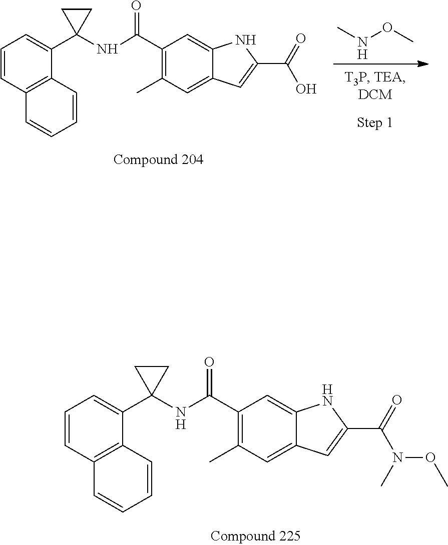

### Step 1: N₂-Methoxy-N_{2,5}-dimethyl-N⁶-(1-(naphthalen-1-yl)cyclopropyl)-1H-indole-2,6-dicarboxamide (Compound 225)

To a solution of 5-methyl-6-((1-(naphthalen-1-yl)cyclopropyl)carbamoyl)-1H-indole-2-carboxylic acid (70.0 mg, 182 μmol, 1.0 eq) and N,O)-dimethylhydroxylamine (26.6 mg, 273 μmol, 1.5 eq, HCl salt) in DCM (3.0 mL) were added TEA (55.3 mg, 546 μmol, 76.0 μL, 3.0 eq) and T₃P (232 mg, 364 μmol, 217 μL, 50% purity in EtOAc, 2.0 eq). The mixture was stirred at 20° C. for 16 h. LCMS indicated that the starting material was completely consumed, and the desired mass was detected. The reaction mixture was poured into H₂O (5.0 mL) and extracted with DCM (5.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 35%-65% B over 8 min; mobile phase A: 0.2% aqueous FA, mobile phase B: acetonitrile). N₂-Methoxy-N²,5-dimethyl-N⁶-(1-(naphthalen-1-yl)cyclopropyl)-1H-indole-2,6-dicarboxamide (18.6 mg, 41.7 μmol, 23% yield) was obtained as a white solid. M+H⁺=428.2 (LCMS); ¹H NMR (400 MHZ, DMSO-d₆) δ 11.5 (s, 1H), 9.1 (s, 1H), 8.7 (d, J=8.19 Hz, 1H), 8.0 (d, J=7.95 Hz, 1H), 7.8-7.9 (m, 2H), 7.5-7.6 (m, 2H), 7.5 (t, J=7.64 Hz, 1H), 7.4 (s, 1H), 7.2 (s, 1H), 7.0 (d, J=1.34 Hz, 1H), 3.8 (s, 3H), 3.3 (s, 3H), 2.1 (s, 3H), 1.4 (s, 2H), 1.2-1.2 (m, 2H).

### Example 135: 5-Methyl-N-(1-(naphthalen-1-yl)cyclopropyl)-2-phenyl-1H-indole-6-carboxamide (Compound 226)

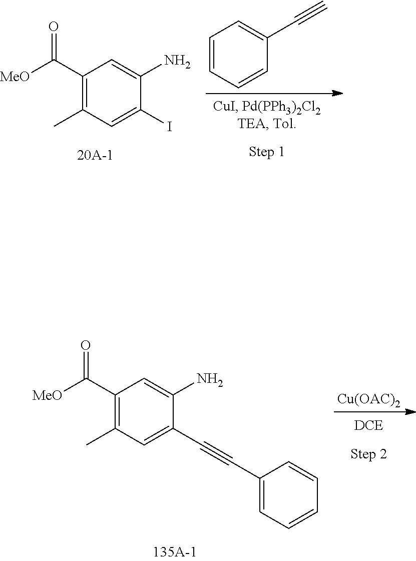

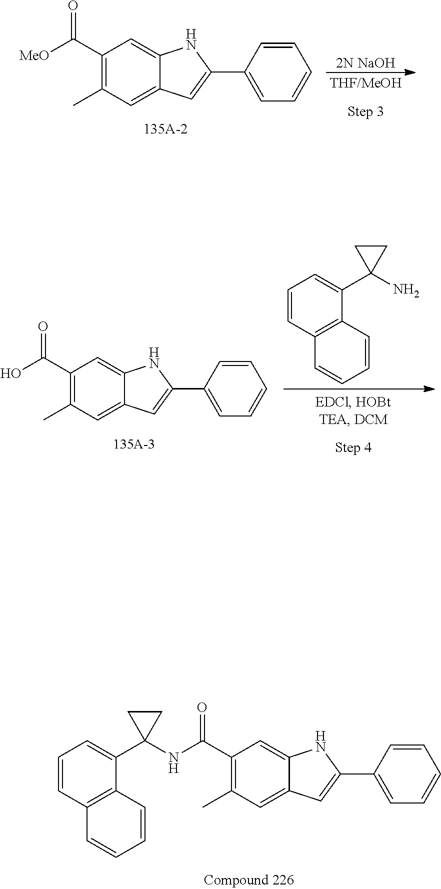

### Step 1: Methyl 5-amino-2-methyl-4-(phenylethynyl)benzoate (135A-1)

A solution of methyl 5-amino-4-iodo-2-methylbenzoate (300 mg, 1.03 mmol, 1.0 eq), ethynylbenzene (158 mg, 1.55 mmol, 170 μL, 1.5 eq) and TEA (209 mg, 2.06 mmol, 287 μL, 2.0 eq) in a mixture of H₂O (7.5 mL) and toluene (15 mL) was degassed and purged with N₂ three times. To the mixture were added Pd(PPh₃)₂Cl₂ (21.7 mg, 30.9 μmol, 0.03 eq) and CuI (1.96 mg, 10.3 μmol, 0.01 eq). The resulting mixture was stirred at 70° C. for 16 h under a N₂ atmosphere. LCMS indicated that the starting material was completely consumed, and the desired mass was detected. The reaction mixture was allowed to cool to room temperature, poured into H₂O (20 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/100 to 1/2. Methyl 5-amino-2-methyl-4-(phenylethynyl)benzoate (250 mg, 942 μmol, 91% yield) was obtained as a brown solid. M+H⁺=266.1 (LCMS); ¹H NMR (400 MHZ, CDCl₃) δ 7.57-7.51 (m, 2H), 7.41-7.35 (m, 3H), 7.33 (s, 1H), 7.25 (s, 1H), 3.89 (s, 3H), 2.47 (s, 3H).

### Step 2: Methyl 5-methyl-2-phenyl-1H-indole-6-carboxylate (135A-2)

A mixture of methyl 5-amino-2-methyl-4-(phenylethynyl)benzoate (216 mg, 814 μmol, 1.0 eq) in DCE (20 mL) was degassed and purged with N₂ three times. To the mixture was added Cu(OAc) 2 (370 mg, 2.04 mmol, 2.5 eq) dropwise at 20° C. The resulting mixture was stirred at 90° C. for 5 h under a N₂ atmosphere. TLC indicated that the starting material was completely consumed. The reaction mixture was allowed to cool to room temperature, poured into H₂O (20 mL) and extracted with EtOAc (10 mL×3). The combined organic layers were dried over Na₂SO₄, filtered, and concentrated under vacuum to give a residue which was purified by preparative TLC (EtOAc/petroleum ether=1/3, R_{f}=0.7). Methyl 5-methyl-2-phenyl-1H-indole-6-carboxylate (150 mg, 565 μmol, 69% yield) was obtained as a brown solid. ¹H NMR (400 MHZ, CDCl₃) δ 8.50 (br s, 1H), 8.10 (s, 1H), 7.51-7.44 (m, 3H), 7.40-7.34 (m, 2H), 7.27-7.24 (m, 1H), 6.77 (d, J=1.1 Hz, 1H), 3.93 (s, 3H), 2.70 (s, 3H).

### Step 3: 5-Methyl-2-phenyl-1H-indole-6-carboxylic acid (135A-3)

To a solution of methyl methyl 5-methyl-2-phenyl-1H-indole-6-carboxylate (70 mg, 264 μmol, 1.0 eq) in a mixture of MeOH (5.0 mL) and THF (5.0 mL) was added NaOH (2 M aqueous, 1.5 mL, 10 eq). The mixture was stirred at 70° C. for 5 h. TLC indicated that the starting material was completely consumed. The reaction mixture was allowed to cool to room temperature, diluted with H₂O (5.0 mL) and washed with MTBE (3.0 mL×3). The aqueous layer was acidified to pH 5 with HCl (1 M aqueous). The product was extracted with EtOAc (5.0 mL×3). The combined organic layers were dried over Na₂SO₄, filtered, and concentrated under vacuum to give the crude product 5-methyl-2-phenyl-1H-indole-6-carboxylic acid (70 mg) as a white solid, which was used in the next step without any further purification.

### Step 4: 5-Methyl-N-(1-(naphthalen-1-yl)cyclopropyl)-2-phenyl-1H-indole-6-carboxamide (Compound 226)

To a solution of 1-(naphthalen-1-yl)cyclopropanamine (58.3 mg, 318 μmol, 1.0 eq) and 5-methyl-2-phenyl-1H-indole-6-carboxylic acid (80.0 mg, 318 μmol, 1.0 eq) in DCM (5 mL) were added TEA (96.7 mg, 955 μmol, 133 μL, 3.0 eq), EDCI (122 mg, 637 μmol, 2.0 eq) and HOBt (86.0 mg, 637 μmol, 2.0 eq). The mixture was stirred at 20° C. for 16 h. LCMS indicated that the starting material was completely consumed, and the desired mass was detected. The reaction mixture was poured into H₂O (5 mL) and extracted with DCM (3.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 45%-75% B over 8 min; mobile phase A: 0.2% aqueous FA, mobile phase B: acetonitrile). 5-Methyl-N-(1-(naphthalen-1-yl)cyclopropyl)-2-phenyl-1H-indole-6-carboxamide (21.1 mg, 50.7 μmol, 16% yield) was obtained as a yellow solid. M+H⁺=417.1 (LCMS); ¹H NMR (400 MHZ, DMSO-d₆) δ 11.47 (s, 1H), 9.09 (s, 1H), 8.73 (d, J=8.5 Hz, 1H), 7.96 (d, J=7.5 Hz, 1H), 7.89-7.79 (m, 4H), 7.65-7.53 (m, 2H), 7.52-7.41 (m, 3H), 7.35-7.24 (m, 2H), 7.11 (s, 1H), 6.80 (d, J=1.4 Hz, 1H), 2.18 (s, 3H), 1.43-1.35 (m, 2H), 1.23-1.16 (m, 2H).

### Example 136: 5-Methyl-N-(1-(naphthalen-1-yl)cyclopropyl)-2-(pyridin-2-yl)-1H-indole-6-carboxamide (Compound 234)

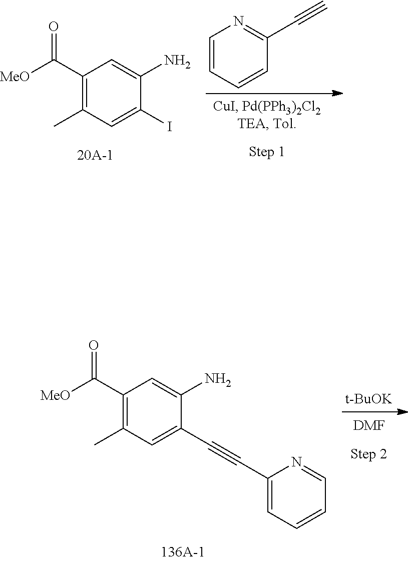

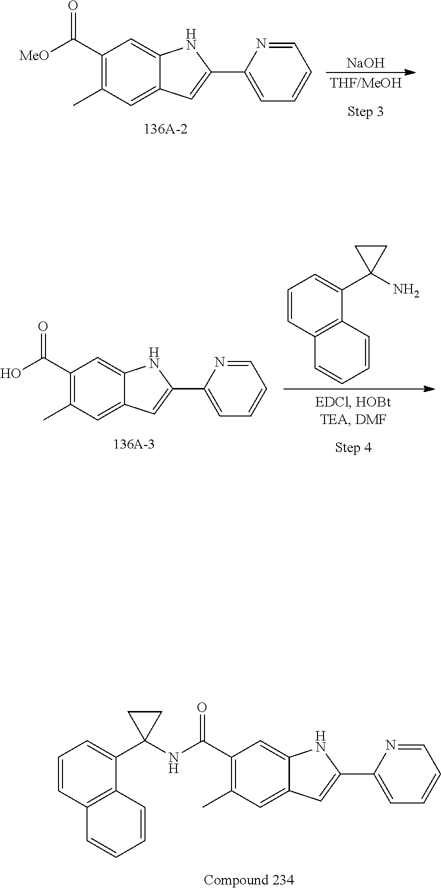

### Step 1: Methyl 5-amino-2-methyl-4-(pyridin-2-ylethynyl)benzoate (136A-1)

A mixture of methyl 5-amino-4-iodo-2-methylbenzoate (1.00 g, 3.44 mmol, 1.0 eq), TEA (695 mg, 6.87 mmol, 956 μL, 2.0 eq), Pd(PPh₃)₂Cl₂ (72.3 mg, 103 μmol, 0.03 eq), CuI (6.54 mg, 34.3 μmol, 0.01 eq) and 2-ethynylpyridine (531 mg, 5.15 mmol, 521 μL, 1.5 eq) in a mixture of toluene (50 mL) and H₂O (25 mL) was degassed and purged with N₂ three times. The resulting mixture was stirred at 70° C. for 3 h under a N₂ atmosphere. TLC indicated that the starting material was completely consumed. The reaction mixture was allowed to cool to room temperature, poured into H₂O (20 mL) and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. Methyl 5-amino-2-methyl-4-(pyridin-2-ylethynyl)benzoate (800 mg, 3.00 mmol, 87% yield) was obtained as a yellow solid.

### Step 2: Methyl 5-methyl-2-(pyridin-2-yl)-1H-indole-6-carboxylate (136A-2)

To the solution of methyl 5-amino-2-methyl-4-(pyridin-2-ylethynyl)benzoate (150 mg, 564 μmol, 1.0 eq) in DMF (7.5 mL) was added 1-BuOK (1 M in THF, 1.18 mL, 2.1 eq). The reaction mixture was stirred at 90° C. for 2 h. LCMS indicated that the starting material was completely consumed, and the desired mass was detected. The reaction mixture was poured into H₂O (10 mL) and extracted with EtOAc (10 mL×3). The combined organic layers were washed with brine (10 mL×2), and then dried over Na₂SO₄, concentrated under vacuum to give methyl 5-methyl-2-(pyridin-2-yl)-1H-indole-6-carboxylate (45.0 mg, 169 μmol, 10% yield) as a yellow gum. M+H⁺=267.2 (LCMS).

### Step 3: 5-Methyl-2-(pyridin-2-yl)-1H-indole-6-carboxylic acid (136A-3)

To a solution of methyl 5-methyl-2-(pyridin-2-yl)-1H-indole-6-carboxylate (45.0 mg, 169 μmol, 1.0 eq) in a mixture of MeOH (3.0 mL) and THF (3.0 mL) was added NaOH (2 M in aqueous, 845 μL, 10 eq). The mixture was stirred at 25° C. for 1 h and then at 70° C. for another 3 h. TLC indicated that the starting material was completely consumed. The reaction mixture was allowed to cool to room temperature, poured into H₂O (5.0 mL) and the pH was adjusted to 6 using HCl (1 M aqueous), and a precipitate was formed. The mixture was filtered, and the filter cake was washed with H₂O (5.0 mL), dried under vacuum to give the crude product 5-methyl-2-(pyridin-2-yl)-1H-indole-6-carboxylic acid (40.0 mg) as a yellow solid.

### Step 4: 5-Methyl-N-(1-(naphthalen-1-yl)cyclopropyl)-2-(pyridin-2-yl)-1H-indole-6-carboxamide (Compound 234)

To a solution of 1-(naphthalen-1-yl)cyclopropanamine (29.1 mg, 159 μmol, 1.0 eq) and 5-methyl-2-(pyridin-2-yl)-1H-indole-6-carboxylic acid (40.0 mg, 157 μmol, 1.0 eq) in DMF (4.0 mL) were added TEA (48.1 mg, 477 μmol, 66.2 μL, 3.0 eq), EDCI (60.8 mg, 317 μmol, 2.0 eq) and HOBt (42.9 mg, 317 μmol, 2.0 eq). The mixture was stirred at 25° C. for 2 h. LCMS indicated that the starting material was completely consumed, and the desired mass was detected. The reaction mixture was poured into H₂O (10 mL) and extracted with DCM (5.0 mL×3). The combined organic layers were washed with brine (5 mL×3), dried over Na₂SO₄, filtered, and concentrated under vacuum to give a residue which was purified by preparative HPLC (Phenomenex Luna C18 column (75×30 mm, 3 μm); flow rate: 25 mL/min; gradient: 30%-70% B over 8 min; mobile phase A: 0.2% aqueous FA, mobile phase B: acetonitrile). 5-Methyl-N-(1-(naphthalen-1-yl)cyclopropyl)-2-(pyridin-2-yl)-1H-indole-6-carboxamide (9.20 mg, 21.5 μmol, 14% yield) was obtained as a yellow solid. M+H⁺=418.1 (LCMS); 1H NMR (400 MHZ, DMSO-d₆) δ 11.63 (d, J=0.9 Hz, 1H), 9.08 (s, 1H), 8.71 (d, J=8.5 Hz, 1H), 8.59 (d, J=4.1 Hz, 1H), 7.95 (dd, J=3.8, 7.7 Hz, 2H), 7.88-7.81 (m, 3H), 7.63-7.51 (m, 2H), 7.51-7.45 (m, 1H), 7.29 (s, 2H), 7.17 (s, 1H), 7.03 (d, J=1.4 Hz, 1H), 2.13 (s, 3H), 1.42-1.34 (m, 2H), 1.21-1.15 (m, 2H).

### Example 137:1,5-Dimethyl-N-(1-(naphthalen-1-yl)cyclopropyl)-1H-indole-6-carboxamide (Compound 201)

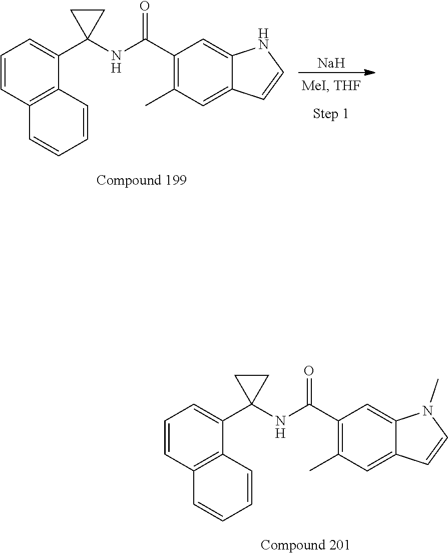

### Step 1:1,5-Dimethyl-N-(1-(naphthalen-1-yl)cyclopropyl)-1H-indole-6-carboxamide (Compound 201)

To a solution of 5-methyl-N-(1-(naphthalen-1-yl)cyclopropyl)-1H-indole-6-carboxamide (100 mg, 294 μmol, 1.0 eq) in THF (4.0 mL) was added sodium hydride (23.5 mg, 294 μmol, 60% purity, 2.0 eq) at 0° C. for 30 min, followed by Mel (41.7 mg, 294 μmol, 9.14 μL, 1.0 eq). The mixture was stirred at 20° C. for 2 h. LCMS indicated that the starting material completely consumed, and the desired mass was detected. The reaction mixture was poured into H₂O (5.0 mL) and extracted with EtOAc (3.0 mL×2). The combined organic layers were dried over Na₂SO₄, filtered, and concentrated under vacuum to give a residue which was purified by preparative HPLC (Phenomenex Luna C18 (80×30 mm, 3 μm); flow rate: 25 mL/min; gradient: 50%-80% B over 8 min; mobile phase A: 0.04% aqueous HCl, mobile phase B: acetonitrile). 1,5-Dimethyl-N-(1-(naphthalen-1-yl)cyclopropyl)-1H-indole-6-carboxamide (60.0 mg, 166 μmol, 63% yield, HCl salt) was obtained as a white solid. M+H⁺=355.0 (LCMS); ¹H NMR (400 MHZ, CDCl₃) δ 8.65-8.57 (m, 1H), 8.16-8.08 (m, 1H), 8.03 (d, J=8.1 Hz, 1H), 7.93 (br d, J=8.4 Hz, 1H), 7.73-7.56 (m, 3H), 7.41 (d, J=18.6 Hz, 2H), 7.14 (d, J=2.0 Hz, 1H), 6.70 (br s, 1H), 6.46 (br s, 1H), 3.79 (s, 3H), 2.40 (s, 3H), 1.72 (br s, 2H), 1.54 (s, 2H).

### Example 138: 1-Cyclobutyl-5-methyl-N-(1-(naphthalen-1-yl)cyclopropyl)-1H-indole-6-carboxamide (Compound 296)

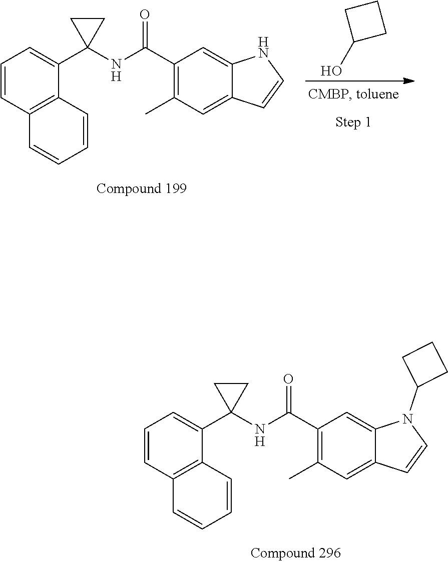 [SMILES: Cc1cc2cc[nH]c2cc1C(=O)NC1(c2cccc3ccccc23)CC1 ; OC1CCC1 ; Cc1cc2ccn(C3CCC3)c2cc1C(=O)NC1(c2cccc3ccccc23)CC1]

### Step 1: 1-Cyclobutyl-5-methyl-N-(1-(naphthalen-1-yl)cyclopropyl)-1H-indole-6-carboxamide (Compound 296)

To a solution of 5-methyl-N-(1-(naphthalen-1-yl)cyclopropyl)-1H-indole-6-carboxamide (80.0 mg, 235 μmol, 1.0 eq) and cyclobutanol (50.8 mg, 705 μmol, 3.0 eq) in toluene (4.0 mL) was added CMBP (56.7 mg, 235 μmol, 1.0 eq). The mixture was stirred at 90° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by preparative HPLC (Phenomenex luna C18 column (80×40 mm, 3 μm); flow rate: 40 mL/min; gradient: 40%-70% B over 7 min; mobile phase A: 0.2% aqueous FA, mobile phase B: acetonitrile) to give 1-cyclobutyl-5-methyl-N-[1-(1-naphthyl)cyclopropyl]indole-6-carboxamide (18.4 mg, 46.3 μmol, 20% yield) as a white solid. M+H⁺=395.1 (LCMS); 1H NMR (400 MHZ, DMSO-d₆) δ 9.01 (s, 1H), 8.72 (d, J=8.3 Hz, 1H), 8.01-7.91 (m, 1H), 7.84 (d, J=7.5 Hz, 2H), 7.66-7.51 (m, 3H), 7.47 (t, J=7.6 Hz, 1H), 7.24 (s, 1H), 7.18 (s, 1H), 6.34 (d, J=3.0 Hz, 1H), 4.83 (quin, J=8.5 Hz, 1H), 2.42-2.27 (m, 4H), 2.11 (s, 3H), 1.86-1.73 (m, 2H), 1.44-1.36 (m, 2H), 1.24-1.14 (m, 2H).

### Example 139: 1-(Azetidin-3-yl)-5-methyl-N-(1-(naphthalen-1-yl)cyclopropyl)-1H-indole-6-carboxamide (Compound 218)

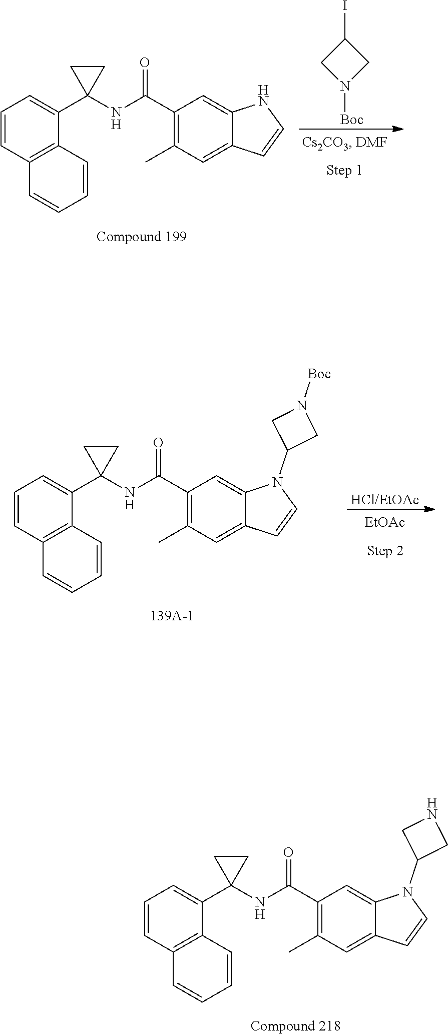

### Step 1: tert-Butyl 3-(5-methyl-6-((1-(naphthalen-1-yl)cyclopropyl)carbamoyl)-1H-indol-1-yl)azetidine-1-carboxylate (139A-1)

To a mixture of 5-methyl-N-(1-(naphthalen-1-yl)cyclopropyl)-1H-indole-6-carboxamide (200 mg, 588 μmol, 1.0 eq), tert-butyl 3-iodoazetidine-1-carboxylate (332 mg, 1.76 mmol, 2.0 eq) and Cs₂CO₃ (384 mg, 1.76 mmol, 2.0 eq) in DMF (2.5 mL) was stirred at 150° C. for 30 min in a microwave (400 W). TLC indicated that 30% of the starting material still remained and 40% of the desired compound was detected. The reaction mixture was allowed to cool to room temperature, poured into H₂O (15 mL) and extracted with EtOAc (10 mL×4). The combined organic layers were washed with brine (10 mL×2), dried over Na₂SO₄, filtered, and concentrated under vacuum to give a residue which was purified by preparative TLC (EtOAc/petroleum ether=1/1, R_{f}=0.6). tert-Butyl 3-(5-methyl-6-((1-(naphthalen-1-yl)cyclopropyl)carbamoyl)-1H-indol-1-yl)azetidine-1-carboxylate (60.0 mg, 100 μmol, 18% yield) was obtained as a yellow gum.

### Step 2: 1-(Azetidin-3-yl)-5-methyl-N-(1-(naphthalen-1-yl)cyclopropyl)-1H-indole-6-carboxamide (Compound 218)

To a solution of tert-butyl 3-(5-methyl-6-((1-(naphthalen-1-yl)cyclopropyl)carbamoyl)-1H-indol-1-yl)azetidine-1-carboxylate (55.0 mg, 111 μmol, 1.0 eq) in DCM (10 mL) was added TFA (2.3 mL). The resulting mixture was stirred at 25° C. for 30 min. LCMS indicated that the starting material was completely consumed, and the desired mass was detected. The reaction mixture was concentrated under vacuum to give a residue which was purified by preparative HPLC (Phenomenex Luna C18 column (150×30 mm, 5 μm); flow rate: 25 mL/min; gradient: 10%-45% B over 8 min; mobile phase A: 0.1% aqueous TFA, mobile phase B: acetonitrile). 1-(Azetidin-3-yl)-5-methyl-N-(1-(naphthalen-1-yl)cyclopropyl)-1H-indole-6-carboxamide (19.7 mg, 38.8 μmol, 35% yield, TFA salt) was obtained as a white solid. M+H⁺=396.1 (LCMS); ¹H NMR (400 MHZ, DMSO-d₆) δ 9.00 (s, 2H), 8.69 (d, J=8.3 Hz, 1H), 7.94 (d, J=7.6 Hz, 1H), 7.88-7.82 (m, 2H), 7.79 (d, J=3.3 Hz, 1H), 7.62-7.45 (m, 3H), 7.30 (s, 1H), 7.25 (s, 1H), 6.52 (d, J=3.1 Hz, 1H), 5.50 (br t, J=7.9 Hz, 1H), 4.50-4.29 (m, 4H), 2.09 (s, 3H), 1.39 (br s, 2H), 1.24-1.16 (m, 2H).

### Example 140: 5-Methyl-1-(1-methylazetidin-3-yl)-N-(1-(naphthalen-1-yl)cyclopropyl)-1H-indole-6-carboxamide (Compound 290)

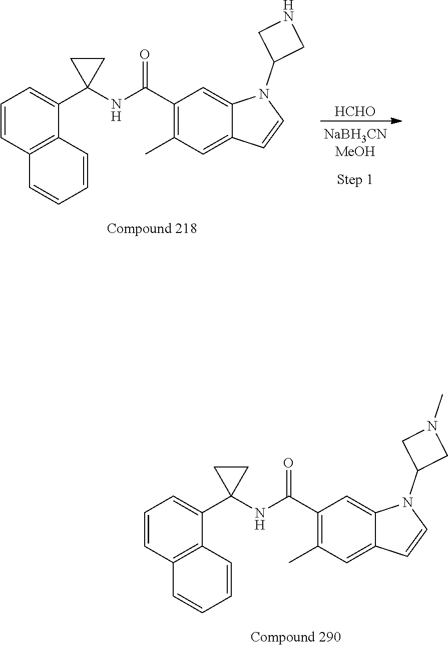 [SMILES: Cc1cc2ccn(C3CNC3)c2cc1C(=O)NC1(c2cccc3ccccc23)CC1 ; "HCHO" ; Cc1cc2ccn(C3CN(C)C3)c2cc1C(=O)NC1(c2cccc3ccccc23)CC1]

### Step 1: 5-Methyl-1-(1-methylazetidin-3-yl)-N-(1-(naphthalen-1-yl)cyclopropyl)-1H-indole-6-carboxamide (Compound 290)

To a solution of 1-(azetidin-3-yl)-5-methyl-N-(1-(naphthalen-1-yl)cyclopropyl)-1H-indole-6-carboxamide (50.0 mg, 83.4 μmol, 1.0 eq, TFA salt) in MeOH (4.0 mL) was added TEA (50 μL), followed by the addition of formaldehyde (27.1 mg, 334 μmol, 24.8 μL, 37% purity in water, 4.0 eq). The resulting mixture was adjusted to pH 6 with a small amount of AcOH. The mixture was stirred at 20° C. for 30 min, then NaBH₃CN (42.0 mg, 667 μmol, 8.0 eq) was added. The resulting reaction mixture was stirred at 20° C. for another 4 h. LCMS indicated that the starting material was completely consumed, and the desired mass was detected. The reaction mixture was poured into H₂O (8.0 mL) and extracted with EtOAc (5.0 mL×4). The combined organic layers were dried over Na₂SO₄, filtered, and concentrated under vacuum to give a residue which was purified by preparative HPLC (Phenomenex Gemini C18 column (100×30 mm, 10 μm); flow rate: 60 mL/min; gradient: 35%-65% B over 8 min; mobile phase A: 10 mM aqueous NH₄HCO₃, mobile phase B: acetonitrile). 5-Methyl-1-(1-methylazetidin-3-yl)-N-(1-(naphthalen-1-yl)cyclopropyl)-1H-indole-6-carboxamide (11.0 mg, 26.6 μmol, 16% yield) was obtained as a yellow solid. M+H⁺=410.1 (LCMS); ¹H NMR (400 MHz, DMSO-d₆) δ 9.02-8.96 (m, 1H), 8.74-8.67 (m, 1H), 7.97-7.91 (m, 1H), 7.88-7.78 (m, 2H), 7.64-7.44 (m, 3H), 7.29-7.20 (m, 2H), 6.41-6.35 (m, 1H), 5.02-4.89 (m, 1H), 3.75-3.60 (m, 2H), 3.30-3.25 (m, 2H), 2.32 (br d, J=2.4 Hz, 3H), 2.16-2.07 (m, 3H), 1.44-1.34 (m, 2H), 1.15 (br s, 2H).

### Example 141: 1-(Azetidin-3-ylmethyl)-5-methyl-N-(1-(naphthalen-1-yl)cyclopropyl)-1H-indole-6-carboxamide (Compound 239)

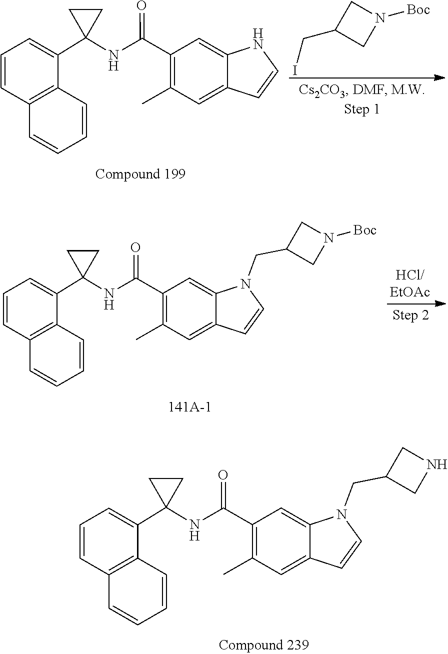

### Step 1: tert-Butyl 3-((5-methyl-6-((1-(naphthalen-1-yl)cyclopropyl)carbamoyl)-1H-indol-1-yl)methyl)azetidine-1-carboxylate (141A-1)

A mixture of 5-methyl-N-(1-(naphthalen-1-yl)cyclopropyl)-1H-indole-6-carboxamide (150 mg, 441 μmol, 1.0 eq), tert-butyl 3-(iodomethyl)azetidine-1-carboxylate (262 mg, 881 μmol, 2.0 eq) and cesium carbonate (287 mg, 881 μmol, 2.0 eq) in DMF (2 mL) was stirred at 150° C. for 30 min in a microwave (400 W). LCMS indicated that 50% starting material remained and 50% desired mass was detected. The mixture reaction was allowed to cool to room temperature, poured into H₂O (5.0 mL) and extracted with EtOAc (1.0 mL×3). The combined organic layers were dried over Na₂SO₄, filtered, and concentrated under vacuum to give a residue which was purified by preparative TLC (EtOAc/petroleum ether=1/1, R_{f}=0.6). tert-Butyl 3-((5-methyl-6-((1-(naphthalen-1-yl)cyclopropyl)carbamoyl)-1H-indol-1-yl)methyl)azetidine-1-carboxylate (120 mg, crude) was obtained as a white solid. M+H⁺=510.3 (LCMS).

### Step 2: 1-(Azetidin-3-ylmethyl)-5-methyl-N-(1-(naphthalen-1-yl)cyclopropyl)-1H-indole-6-carboxamide (Compound 239)

To a stirred solution of tert-butyl 3-((5-methyl-6-((1-(naphthalen-1-yl)cyclopropyl)carbamoyl)-1H-indol-1-yl)methyl)azetidine-1-carboxylate (120 mg, 235 μmol, 1.0 eq) in DCM (10 mL) was added TFA (2.0 mL). The mixture was stirred at 20° C. for 30 min. LCMS indicated that the starting material was completely consumed, and the desired mass was detected. The mixture was concentrated under vacuum at 30° C. to give a residue which was purified by preparative HPLC (Phenomenex Luna C18 column (100×30 mm, 5 μm); flow rate: 25 mL/min; gradient: 25%-55% B over 8 min; mobile phase A: 0.1% aqueous TFA, mobile phase B: acetonitrile). 1-(Azetidin-3-ylmethyl)-5-methyl-N-(1-(naphthalen-1-yl)cyclopropyl)-1H-indole-6-carboxamide (22.9 mg, 43.7 μmol, 6% yield, TFA salt) was obtained as a white solid. M+H⁺=410.1 (LCMS); ¹H NMR (400 MHZ, DMSO-d₆) δ 9.00 (s, 1H), 8.68 (d, J=8.4 Hz, 1H), 8.61 (br dd, J=2.5, 4.1 Hz, 1H), 8.55-8.44 (m, 1H), 7.98-7.91 (m, 1H), 7.90-7.80 (m, 2H), 7.60-7.44 (m, 3H), 7.38 (d, J=3.1 Hz, 1H), 7.27 (s, 2H), 6.35 (d, J=3.0 Hz, 1H), 4.34 (d, J=7.4 Hz, 2H), 3.94-3.80 (m, 2H), 3.75-3.63 (m, 2H), 2.08 (s, 3H), 1.43-1.35 (m, 2H), 1.24-1.15 (m, 2H).

### Example 142: 5-Methyl-N-(1-(naphthalen-1-yl)cyclopropyl)-1-(oxetan-3-ylmethyl)-1H-indole-6-carboxamide (Compound 295)

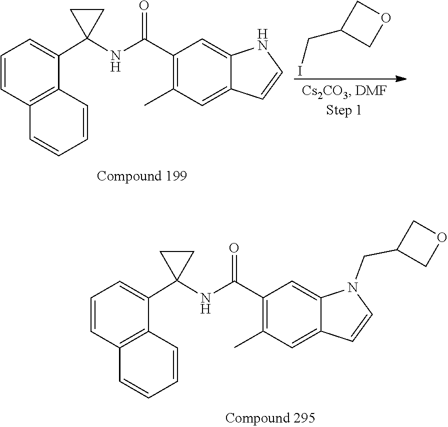

### Step 1: 5-Methyl-N-(1-(naphthalen-1-yl)cyclopropyl)-1-(oxetan-3-ylmethyl)-1H-indole-6-carboxamide (Compound 295)

To a solution of 5-methyl-N-(1-(naphthalen-1-yl)cyclopropyl)-1H-indole-6-carboxamide (80.0 mg, 235 μmol, 1.0 eq) in DMF (4.0 mL) were added Cs₂CO₃ (230 mg, 705 μmol, 3.0 eq) and 3-(iodomethyl)oxetane (93.1 g, 470 μmol, 2.0 eq). The mixture was stirred at 100° C. for 16 h. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by preparative HPLC (Phenomenex luna C18 column (80×40 mm, 3 μm); flow rate: 40 mL/min; gradient: 43%-73% B over 7 min; mobile phase A: 0.2% aqueous FA, mobile phase B: acetonitrile) to give 5-methyl-N-(1-(naphthalen-1-yl)cyclopropyl)-1-(oxetan-3-ylmethyl)-1H-indole-6-carboxamide (39.3 mg, 95.4 μmol, 41% yield) as a white solid. M+H⁺=411.1 (LCMS); ¹H NMR (400 MHZ, DMSO-d₆) δ 9.00 (s, 1H), 8.71 (d, J=8.4 Hz, 1H), 7.98-7.90 (m, 1H), 7.84 (dd, J=2.3, 7.7 Hz, 2H), 7.61-7.45 (m, 3H), 7.40 (d, J=3.0 Hz, 1H), 7.23 (d, J=12.8 Hz, 2H), 6.30 (d, J=2.8 Hz, 1H), 4.56 (dd, J=6.1, 7.8 Hz, 2H), 4.44-4.30 (m, 4H), 3.42-3.34 (m, 1H), 2.09 (s, 3H), 1.46-1.37 (m, 2H), 1.24-1.15 (m, 2H).

### Example 143: 1-(2-Aminoethyl)-5-methyl-N-(1-(naphthalen-1-yl)cyclopropyl)-1H-indole-6-carboxamide (Compound 203)

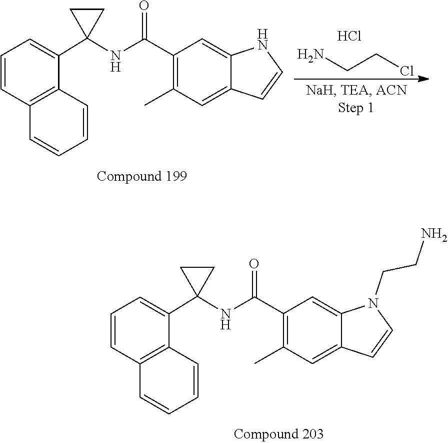

### Step 1: 1-(2-Aminoethyl)-5-methyl-N-(1-(naphthalen-1-yl)cyclopropyl)-1H-indole-6-carboxamide (Compound 203)

To a mixture of 5-methyl-N-(1-(naphthalen-1-yl)cyclopropyl)-1H-indole-6-carboxamide (80.0 mg, 235 μmol, 1.0 eq) in acetonitrile (4.0 mL) was added sodium hydride (18.8 mg, 470 μmol, 60% purity, 2.0 eq). The mixture was degassed and purged with N₂ three times and stirred at 0° C. for 10 min. To the mixture was added 2-chloroethanamine (27.3 mg, 235 μmol, 1.0 eq, HCl salt), followed by TEA (23.8 mg, 235 μmol, 32.7 μL, 1.0 eq). The mixture was stirred at 20° C. for 2 h under a N₂ atmosphere. LCMS indicated that the starting material was completely consumed, and the desired mass was detected. The reaction mixture was poured into H₂O (4.0 mL) and extracted with EtOAc (2.0 mL×3). The combined organic layers were dried over Na₂SO₄, filtered, and concentrated under vacuum to give a residue which was purified by preparative HPLC (Phenomenex Luna (80×30 mm, 3 μm); flow rate: 25 mL/min; gradient: 15%-45% B over 8 min; mobile phase A: 0.04% aqueous HCl, mobile phase B: acetonitrile). 1-(2-Aminoethyl)-5-methyl-N-(1-(naphthalen-1-yl)cyclopropyl)-1H-indole-6-carboxamide (10.6 mg, 25.2 μmol, 11% yield, HCl salt) was obtained as a white solid. M+H⁺=384.1 (LCMS); ¹H NMR (400 MHZ, DMSO-d₆) δ 9.02 (s, 1H), 8.70 (d, J=8.2 Hz, 1H), 7.95-7.82 (m, 5H), 7.60-7.45 (m, 3H), 7.38 (d, J=3.1 Hz, 1H), 7.29 (d, J=9.4 Hz, 2H), 6.38 (d, J=2.9 Hz, 1H), 4.30 (t, J=6.8 Hz, 2H), 3.16-3.09 (m, 1H), 3.18-3.08 (m, 1H), 2.10 (s, 3H), 1.44-1.36 (m, 2H), 1.22-1.15 (m, 2H).

### Example 144: 3,5-Dimethyl-N-(1-(naphthalen-1-yl)cyclopropyl)-1H-indole-6-carboxamide (Compound 212)

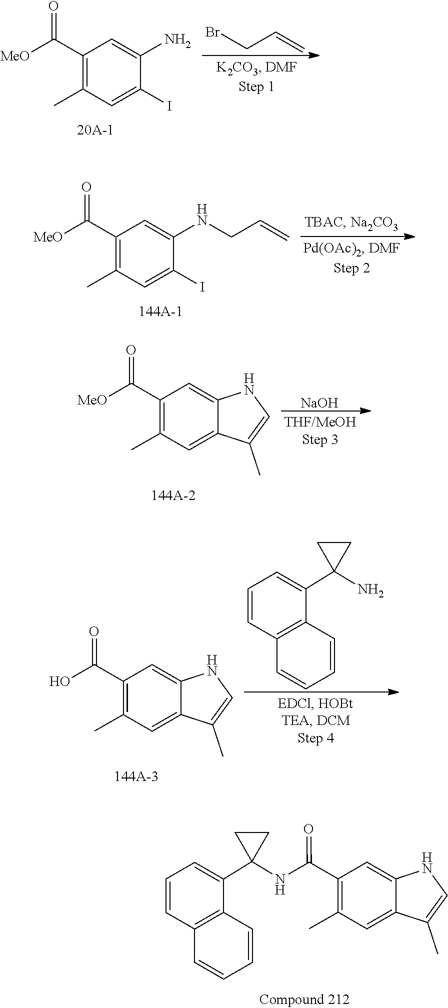

### Step 1: Methyl 5-(allylamino)-4-iodo-2-methylbenzoate (144A-1)

To a solution of methyl 5-amino-4-iodo-2-methylbenzoate (300 mg, 1.03 mmol, 1.0 eq) and K₂CO₃ (427 mg, 3.09 mmol, 3.0 eq) in DMF (5.0 mL) was added 3-bromoprop-1-ene (125 mg, 1.03 mmol, 1.0 eq) slowly. The mixture was stirred at 25° C. for 60 h. LCMS indicated that the starting material was completely consumed, and the desired mass was detected. The reaction mixture was poured into H₂O (15 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/100 to 1/5. Methyl 5-(allylamino)-4-iodo-2-methylbenzoate (103 mg, 311 μmol, 30% yield) was obtained as a white oil. M+H⁺=331.9 (LCMS).

### Step 2: Methyl 3,5-dimethyl-1H-indole-6-carboxylate (144A-2)

To a solution of methyl 5-(allylamino)-4-iodo-2-methylbenzoate (103 mg, 311 μmol, 1.0 eq) in DMF (8.0 mL) were added Na₂CO₃ (82.4 mg, 778 μmol, 2.5 eq), TBAC (95.1 mg, 342 μmol, 95.7 μL, 1.1 eq) and Pd(OAc) 2 (6.98 mg, 31.1 μmol, 0.1 eq). The resulting mixture was stirred at 100° C. for 2 h under a N₂ atmosphere. TLC indicated that the starting material was completely consumed. The reaction mixture was allowed to cool to room temperature, poured into H₂O (15 mL) and extracted with EtOAc (10 mL×3). The combined organic layers were dried over Na₂SO₄, filtered, and concentrated under vacuum to give a residue which was purified by preparative TLC (EtOAc/petroleum ether=1/5, R_{f}=0.5). Methyl 3,5-dimethyl-1H-indole-6-carboxylate (30.0 mg, 148 μmol, 47% yield) was obtained as a brown solid.

### Step 3: 3,5-Dimethyl-1H-indole-6-carboxylic acid (144A-3)

To a solution of methyl 3,5-dimethyl-1H-indole-6-carboxylate (70.0 mg, 344 μmol, 1.0 eq) in a mixture of THF (3.5 mL) and MeOH (3.5 mL) was added NaOH (2 M aqueous, 1.72 mL, 10 eq). The mixture was stirred at 25° C. for 1 h then at 70° C. for another 1 h. TLC indicated that the starting material was completely consumed. The reaction mixture was allowed to cool to room temperature, poured into H₂O (3.0 mL) and extracted with MTBE (5.0 mL×2). The aqueous was acidified to pH 5 using HCl (1 M aqueous). The product was extracted with EtOAc (5.0 mL×5) and the combined organic layers were dried over Na₂SO₄, filtered, and concentrated under vacuum to give the crude product 3,5-dimethyl-1H-indole-6-carboxylic acid (60.0 mg, 317 μmol, 92% yield) as a white solid.

### Step 4: 3,5-Dimethyl-N-(1-(naphthalen-1-yl)cyclopropyl)-1H-indole-6-carboxamide (Compound 212)

To a solution of 1-(naphthalen-1-yl)cyclopropanamine (58.1 mg, 317 μmol, 1.0 eq) and 3,5-dimethyl-1H-indole-6-carboxylic acid (60.0 mg, 317 μmol, 1.0 eq) in DCM (6.0 mL) were added TEA (96.3 mg, 951 μmol, 132 μL, 3.0 eq), EDCI (91.2 mg, 476 μmol, 1.5 eq) and HOBt (64.3 mg, 476 μmol, 1.5 eq). The mixture was stirred at 25° C. for 2 h. LCMS indicated that the starting material was completely consumed, and the desired mass was detected. The reaction mixture was poured into H₂O (10 mL) and extracted with DCM (10 mL×2). The combined organic layers were dried over Na₂SO₄, filtered, and concentrated under vacuum to give a residue which was purified by preparative HPLC (Phenomenex luna C18 column (100×40 mm, 3 μm); flow rate: 25 mL/min; gradient: 55%-75% B over 7 min; mobile phase A: 0.04% aqueous HCl, mobile phase B: acetonitrile). 3,5-Dimethyl-N-(1-(naphthalen-1-yl)cyclopropyl)-1H-indole-6-carboxamide (32.1 mg, 81.9 μmol, 26% yield, HCl salt) was obtained as a white solid. M+H⁺=355.1 (LCMS); ¹H NMR (400 MHZ, CDCl₃) δ 8.48 (br d, J=8.7 Hz, 1H), 7.98 (d, J=6.8 Hz, 1H), 7.91 (d, J=7.8 Hz, 1H), 7.81 (br d, J=8.2 Hz, 2H), 7.64-7.55 (m, 1H), 7.48 (s, 2H), 7.14 (s, 1H), 6.95 (s, 1H), 6.54 (br s, 1H), 2.33 (s, 3H), 2.26 (s, 3H), 1.58-1.56 (m, 2H), 1.41 (s, 2H).

### Example 145: 3-(2-Aminoethyl)-5-methyl-N-(1-(naphthalen-1-yl)cyclopropyl)-1H-indole-6-carboxamide (Compound 292)

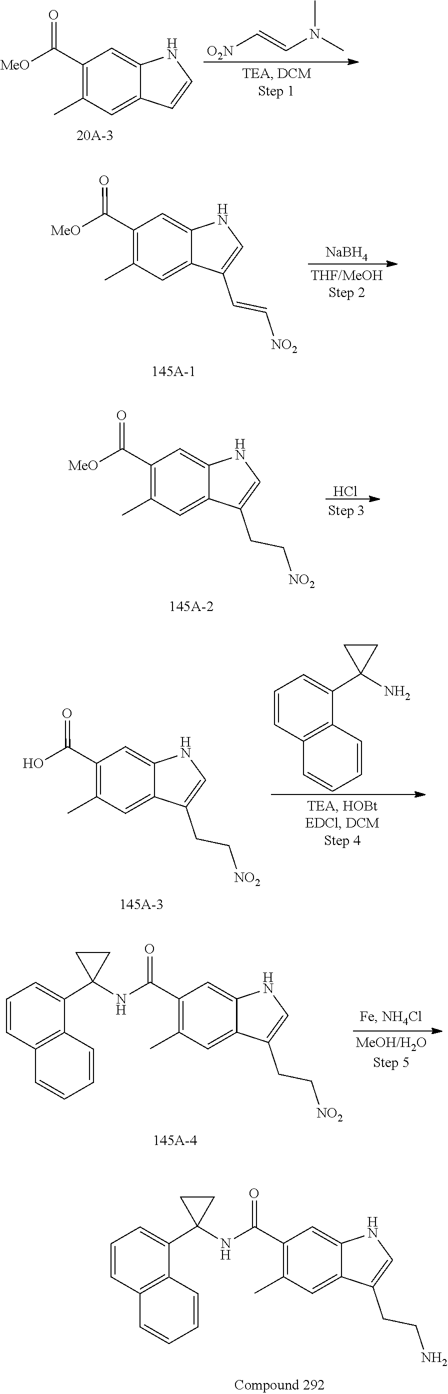

### Step 1: (E)-Methyl 5-methyl-3-(2-nitrovinyl)-1H-indole-6-carboxylate (145A-1)

To a solution of methyl 5-methyl-1H-indole-6-carboxylate (460 mg, 2.43 mmol, 1.0 eq) and (E)-N,N-dimethyl-2-nitroethenamine (282 mg, 2.43 mmol, 1.0 eq) in DCM (5.0 mL) was added TFA (277 mg, 2.43 mmol, 0.18 mL, 1.0 eq). The mixture was stirred at 20° C. for 5 h. TLC indicated that the starting material was completely consumed. The reaction mixture was poured into H₂O (5.0 mL) and extracted with EtOAc (3.0 mL×2). The combined organic layers were dried over Na₂SO₄, filtered, and concentrated under vacuum to give a residue which was purified by trituration from EtOAc (5 mL). (E)-Methyl 5-methyl-3-(2-nitrovinyl)-1H-indole-6-carboxylate (300 mg, 1.15 mmol, 47% yield) was obtained as a yellow solid. M+H⁺=261.0 (LCMS); ¹H NMR (400 MHZ, CD₃OD) δ 8.40-8.33 (m, 1H), 8.09 (s, 1H), 8.04 (s, 1H), 7.95-7.89 (m, 1H), 7.72 (s, 1H), 3.90 (s, 3H), 2.71 (s, 3H).

### Step 2: Methyl 5-methyl-3-(2-nitroethyl)-1H-indole-6-carboxylate (145A-2)

To a solution of (E)-methyl 5-methyl-3-(2-nitrovinyl)-1H-indole-6-carboxylate (300 mg, 1.15 mmol, 1.0 eq) in a mixture of THF (15 mL) and MeOH (5.0 mL) was added NaBH₄ (131 mg, 3.46 mmol, 3.0 eq) at 0° C. Then the mixture was stirred at 20° C. for 16 h. LCMS indicated that starting material was completely consumed, and the desired mass was detected. The reaction mixture was poured into H₂O (20 mL) and extracted with DCM (10 mL×2). The combined organic layers were dried over Na₂SO₄, filtered, and concentrated under vacuum to give a residue which was purified by flash silica gel chromatography using a gradient of EtOAc/petroleum ether from 0/1 to 1/5. Methyl 5-methyl-3-(2-nitroethyl)-1H-indole-6-carboxylate (230 mg, 877 μmol, 76% yield) was obtained as a white solid. M+H⁺=263.1 (LCMS); ¹H NMR (400 MHZ, CDCl₃) δ 8.21-8.11 (m, 1H), 8.09-8.04 (m, 1H), 7.41-7.37 (m, 1H), 7.22-7.17 (m, 1H), 4.79-4.53 (m, 2H), 4.01-3.77 (m, 3H), 3.64-3.35 (m, 2H), 2.83-2.59 (m, 3H).

### Step 3: 5-Methyl-3-(2-nitroethyl)-1H-indole-6-carboxylic acid (145A-3)

A solution of methyl 5-methyl-3-(2-nitroethyl)-1H-indole-6-carboxylate (70.0 mg, 267 μmol, 1.0 eq) in HCl (2 M aqueous, 2.0 mL) was stirred at 110° C. for 8 h. LCMS indicated that the starting material was completely consumed, and the desired mass was detected. The reaction mixture was allowed to cool to room temperature, treated with NaOH (2 M aqueous) to adjust the pH to 6. The mixture was concentrated under vacuum to remove the water completely. The mixture was treated with MeOH/DCM (V/V=10/1, 2.0 mL) then filtered. The filter cake was washed with MeOH/DCM (V/V=10/1, 2.0 mL×2) to ensure all product was washed from the solids. The combined organic layers were concentrated under vacuum to give crude product 5-methyl-3-(2-nitroethyl)-1H-indole-6-carboxylic acid (100 mg, 80% purity) as a grey solid. M+H⁺=249.1 (LCMS).

### Step 4: 5-Methyl-N-(1-(naphthalen-1-yl)cyclopropyl)-3-(2-nitroethyl)-1H-indole-6-carboxamide (145A-4)

To a solution of 5-methyl-3-(2-nitroethyl)-1H-indole-6-carboxylic acid (100 mg, 403 μmol, 1.0 eq) and 1-(naphthalen-1-yl)cyclopropanamine (73.8 mg, 403 μmol, 1.0 eq) in DCM (3.0 mL) were added TEA (81.5 mg, 806 μmol, 0.12 mL, 2.0 eq), EDCI (92.7 mg, 483 μmol, 1.2 eq) and HOBt (65.3 mg, 483 μmol, 1.2 eq). The mixture was stirred at 20° C. for 16 h. LCMS indicated that starting material was completely consumed, and the desired mass was detected. The reaction mixture was poured into H₂O (3.0 mL) and extracted with DCM (3.0 mL×2). The combined organic layers were dried over Na₂SO₄, filtered, and concentrated under vacuum to give a residue which was purified by preparative TLC (EtOAc/petroleum ether=1/1, R_{f}=0.4). 5-Methyl-N-(1-(naphthalen-1-yl)cyclopropyl)-3-(2-nitroethyl)-1H-indole-6-carboxamide (15.0 mg, 36.3 μmol, 9% yield) was obtained as a white solid. M+H⁺=414.2 (LCMS).

### Step 5:3-(2-Aminoethyl)-5-methyl-N-(1-(naphthalen-1-yl)cyclopropyl)-1H-indole-6-carboxamide (Compound 292)

To a solution of 5-methyl-N-(1-(naphthalen-1-yl)cyclopropyl)-3-(2-nitroethyl)-1H-indole-6-carboxamide (15.0 mg, 36.3 μmol, 1.0 eq) in a mixture of MeOH (2.0 mL) and H₂O (0.5 mL) were added iron powder (10.1 mg, 181 μmol, 5.0 eq) and NH₄Cl (9.70 mg, 181 μmol, 5.0 eq). The mixture was stirred at 80° C. for 2 h. LCMS indicated that starting material was completely consumed, and the desired mass was detected. The reaction mixture was allowed to cool to room temperature, poured into H₂O (4.0 mL) the mixture was filtered through a pad of Celite and the slurry was washed with EtOAc several times and extracted with EtOAc (2.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 25%-40% B over 8 min; mobile phase A: 0.04% aqueous HCl, mobile phase B: acetonitrile). 3-(2-Aminoethyl)-5-methyl-N-(1-(naphthalen-1-yl)cyclopropyl)-1H-indole-6-carboxamide (1.2 mg, 3.13 μmol, 9% yield, HCl salt) was obtained as a white solid. M+H⁺=384.0 (LCMS); ¹H NMR (400 MHZ, CD₃OD) δ 8.64 (d, J=8.5 Hz, 1H), 7.94-7.88 (m, 2H), 7.81 (d, J=8.3 Hz, 1H), 7.61-7.55 (m, 1H), 7.53-7.42 (m, 2H), 7.31 (s, 1H), 7.17 (s, 1H), 7.14 (s, 1H), 3.16 (br d, J=7.3 Hz, 2H), 3.05 (d, J=7.4 Hz, 2H), 2.21 (s, 3H), 1.52-1.42 (m, 2H), 1.31 (br d, J=1.9 Hz, 2H).

### Example 146: 5-Methyl-N-(1-(naphthalen-1-yl)cyclopropyl)indoline-6-carboxamide (Compound 207)

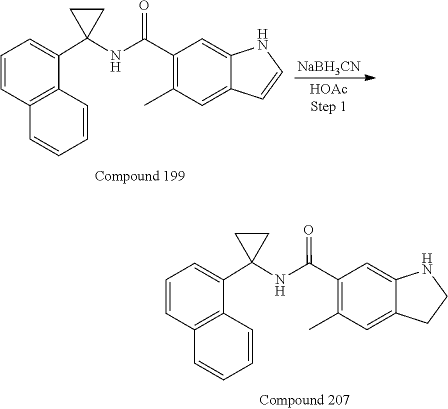

### Step 1: 5-Methyl-N-(1-(naphthalen-1-yl)cyclopropyl)indoline-6-carboxamide (Compound 207)

To a solution of 5-methyl-N-(1-(naphthalen-1-yl)cyclopropyl)-1H-indole-6-carboxamide (100 mg, 294 μmol, 1.0 eq) in AcOH (10 mL) was added NaBH₃CN (36.9 mg, 588 μmol, 2.0 eq), the resulting mixture was stirred at 25° C. for 16 h. LCMS indicated that the starting material was completely consumed, and the desired mass was detected. The mixture was poured into H₂O (10 mL) and extracted with DCM (5.0 mL×5). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 20%-60% B over 8 min; mobile phase A: 0.04% aqueous HCl, mobile phase B: acetonitrile). 5-Methyl-N-(1-(naphthalen-1-yl)cyclopropyl)indoline-6-carboxamide (40.0 mg, 116 μmol, 40% yield) was obtained as a white solid. M+H⁺=343.0 (LCMS); ¹H NMR (400 MHz, DMSO-d₆) δ 9.19 (s, 1H), 8.62 (d, J=8.4 Hz, 1H), 7.94 (d, J=7.5 Hz, 1H), 7.88-7.79 (m, 2H), 7.61-7.44 (m, 3H), 7.16 (s, 1H), 6.92 (s, 1H), 3.60 (br t, J=7.9 Hz, 2H), 3.06 (br t, J=7.7 Hz, 2H), 2.00 (s, 3H), 1.36 (br d, J=1.5 Hz, 2H), 1.21-1.15 (m, 2H).

### Example 147: 1,5-Dimethyl-N-(1-(naphthalen-1-yl)cyclopropyl)indoline-6-carboxamide (Compound 210)

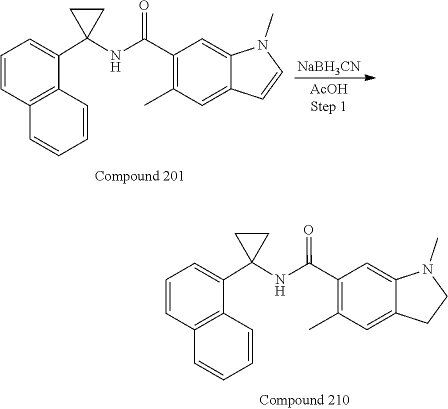

### Step 1: 1,5-Dimethyl-N-(1-(naphthalen-1-yl)cyclopropyl)indoline-6-carboxamide (Compound 210)

To a solution of 1,5-dimethyl-N-(1-(naphthalen-1-yl)cyclopropyl)-1H-indole-6-carboxamide (50.0 mg, 141 μmol, 1.0 eq) in AcOH (1.0 mL) was added NaBH₃CN (17.7 mg, 282 μmol, 2.0 eq). The resulting mixture was stirred at 20° C. for 16 h. LCMS indicated that the starting material completely consumed, and the desired mass was detected. The reaction mixture was poured into H₂O (3.0 mL) and extracted with EtOAc (2.0 mL×2). The combined organic layers were dried over Na₂SO₄, filtered, and concentrated under vacuum to give a residue which was purified by preparative HPLC (Phenomenex Luna C18 (80×30 mm, 3 μm); flow rate: 25 mL/min; gradient: 10%-40% B over 8 min; mobile phase A: 0.04% aqueous HCl, mobile phase B: acetonitrile). 1,5-Dimethyl-N-(1-(naphthalen-1-yl)cyclopropyl)indoline-6-carboxamide (31.7 mg, 80.7 μmol, 57% yield, HCl salt) was obtained as a white solid. M+H⁺=357.1 (LCMS); ¹H NMR (400 MHZ, DMSO-d₆) δ 9.04-8.97 (m, 1H), 8.66 (d, J=8.3 Hz, 1H), 7.96-7.90 (m, 1H), 7.82 (t, J=6.7 Hz, 2H), 7.46 (s, 3H), 6.98-6.79 (m, 1H), 6.55-6.35 (m, 1H), 3.33-3.25 (m, 2H), 2.89-2.81 (m, 2H), 2.70-2.66 (m, 3H), 1.91 (s, 3H), 1.35 (br d, J=1.0 Hz, 2H), 1.17 (s, 2H).

### Example 148: 5-Methyl-N-(1-(naphthalen-1-yl)cyclopropyl)-2-oxoindoline-6-carboxamide (Compound 249)

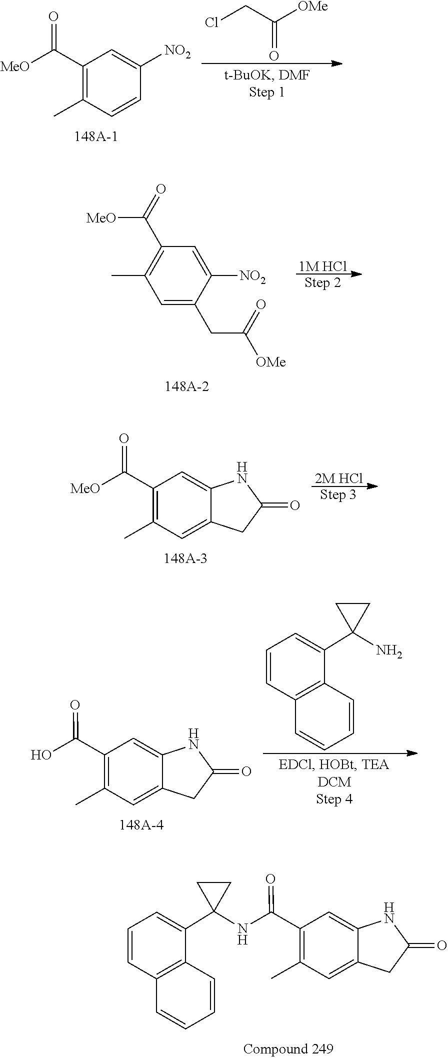

### Step 1: Methyl 4-(2-methoxy-2-oxoethyl)-2-methyl-5-nitrobenzoate (148A-2)

To a solution of methyl 2-methyl-5-nitrobenzoate (5.00 g, 25.6 mmol, 1.0 eq) and methyl 2-chloroacetate (3.06 g, 28.2 mmol, 1.1 eq) in DMF (50 mL) was slowly added a solution of t-BuOK (7.19 g, 64.1 mmol, 2.5 eq) in DMF (100 mL) at 0° C. and the mixture was stirred at 0° C. for 3 h. LCMS indicated that the starting material was completely consumed, and the desired mass was detected. The reaction mixture was poured into H₂O (100 mL) and extracted with EtOAc (50 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. Methyl 4-(2-methoxy-2-oxoethyl)-2-methyl-5-nitrobenzoate (4.00 g, 15.0 mmol, 29% yield) was obtained as a yellow solid. M+H⁺=268.1 (LCMS); ¹H NMR (400 MHZ, CDCl₃) δ 8.73 (s, 1H), 7.25 (s, 1H), 4.05 (s, 2H), 3.95 (s, 3H), 3.73 (s, 3H), 2.70 (s, 3H).

### Step 2: Methyl 5-methyl-2-oxoindoline-6-carboxylate (148A-3)

A solution of methyl 4-(2-methoxy-2-oxoethyl)-2-methyl-5-nitrobenzoate (400 mg, 1.69 mmol, 1.0 eq) in HCl (1 M aqueous, 16 mL, 9.5 eq) was stirred at 100° C. for 1 h. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. Methyl 5-methyl-2-oxoindoline-6-carboxylate (200 mg, 975 μmol, 58% yield) was obtained as a brown solid. M+H⁺=206.1 (LCMS); ¹H NMR (400 MHZ, DMSO-d₆) δ 10.43 (s, 1H), 7.22 (s, 1H), 7.17 (s, 1H), 3.81 (s, 3H), 3.51 (s, 2H), 2.45 (s, 3H).

### Step 3: 5-Methyl-2-oxoindoline-6-carboxylic acid (148A-4)

A solution of methyl 5-methyl-2-oxoindoline-6-carboxylate (150 mg, 731 μmol, 1.0 eq) in HCl (2 M aqueous, 5.0 mL) was stirred at 100° C. for 16 h. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give the crude product 5-methyl-2-oxoindoline-6-carboxylic acid (100 mg), which was used in the next step without any further purification. M+H⁺=192.0 (LCMS); ¹H NMR (400 MHZ, DMSO-d₆) δ 10.39 (s, 1H), 7.24 (s, 1H), 7.13 (s, 1H), 3.49 (s, 2H), 2.46 (s, 3H).

### Step 4: 5-Methyl-N-(1-(naphthalen-1-yl)cyclopropyl)-2-oxoindoline-6-carboxamide (Compound 249)

To a solution of 1-(naphthalen-1-yl)cyclopropanamine (47.9 mg, 262 μmol, 1.0 eq) and 5-methyl-2-oxoindoline-6-carboxylic acid (50.0 mg, 262 μmol, 1.0 eq) in DCM (5.0 mL) were added TEA (79.4 mg, 785 μmol, 109 μL, 3.0 eq), EDCI (100 mg, 523 μmol, 2.0 eq) and HOBt (70.7 mg, 523 mmol, 2.0 eq). The mixture was stirred at 20° C. for 16 h. LCMS indicated that the starting material was completely consumed, and the desired mass was detected. The reaction mixture was poured into H₂O (10 mL) and extracted with DCM (3.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 30%-50% B over 8 min; mobile phase A: 0.2% aqueous FA, mobile phase B: acetonitrile). 5-Methyl-N-(1-(naphthalen-1-yl)cyclopropyl)-2-oxoindoline-6-carboxamide (13.5 mg, 37.9 μmol, 14% yield, FA salt) was obtained as a white solid. M+H⁺=357.0 (LCMS); ¹H NMR (400 MHZ, DMSO-d₆) δ 10.26 (s, 1H), 9.10 (s, 1H), 8.64 (d, J=8.4 Hz, 1H), 7.93 (d, J=8.1 Hz, 1H), 7.82 (t, J=7.4 Hz, 2H), 7.61-7.42 (m, 3H), 6.96 (s, 1H), 6.46 (s, 1H), 3.40 (s, 2H), 1.98 (s, 3H), 1.33 (s, 2H), 1.19-1.13 (m, 2H).

### Example 149: 3,3,5-Trimethyl-N-(1-(naphthalen-1-yl)cyclopropyl)-2-oxoindoline-6-carboxamide (Compound 243)

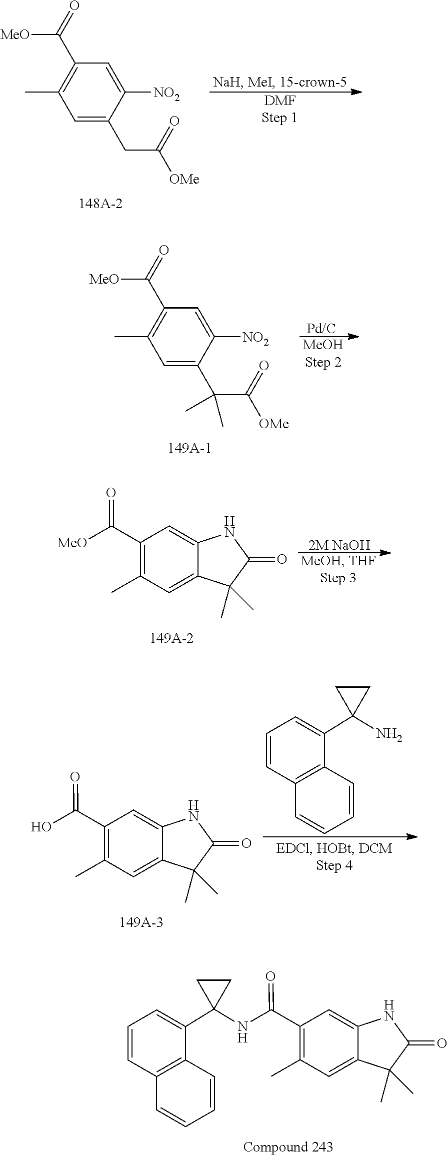

### Step 1: Methyl 4-(1-methoxy-2-methyl-1-oxopropan-2-yl)-2-methyl-5-nitrobenzoate (149A-1)

To a solution of methyl 4-(2-methoxy-2-oxoethyl)-2-methyl-5-nitrobenzoate (500 mg, 1.87 mmol, 1.0 eq) in DMF (50 mL) was slowly added Mel (664 mg, 4.68 mmol, 291 μL, 2.5 eq), 15-crown-5 (103 mg, 468 μmol, 92.8 μL, 0.25 eq) and sodium hydride (187 mg, 4.68 mmol, 60% purity, 2.5 eq) at 0° C. The mixture was stirred at 0° C. for 1 h. LCMS indicated that the starting material was completely consumed, and the desired mass was detected. The reaction mixture was poured into H₂O (30 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. Methyl 4-(1-methoxy-2-methyl-1-oxopropan-2-yl)-2-methyl-5-nitrobenzoate (600 mg, crude) was obtained as a white solid. M+H⁺=296.0 (LCMS); ¹H NMR (400 MHZ, CDCl₃) δ 8.54 (s, 1H), 7.46 (s, 1H), 3.94 (s, 3H), 3.66 (s, 3H), 2.73 (s, 3H), 1.68 (s, 6H).

### Step 2: Methyl 3,3,5-trimethyl-2-oxoindoline-6-carboxylate (149A-2)

To a solution of methyl 4-(1-methoxy-2-methyl-1-oxopropan-2-yl)-2-methyl-5-nitrobenzoate (300 mg, 1.02 mmol, 1.0 eq) in MeOH (60 mL) was added 10% palladium on carbon (300 mg). The mixture was degassed and purged with H₂ three times, and then the mixture was stirred at 50° C. for 16 h under a H₂ (50 psi) atmosphere. LCMS indicated that the starting material was completely consumed, and the desired mass was detected. The reaction mixture was allowed to cool to room temperature and filtered through a pad of Celite. The filtrate was concentrated under vacuum to give the crude product methyl 3,3,5-trimethyl-2-oxoindoline-6-carboxylate (200 mg, 857 μmol, 84% yield) as a yellow oil. M+H⁺=234.2 (LCMS); ¹H NMR (400 MHZ, CDCl₃) δ 8.06 (br s, 1H), 7.48 (s, 1H), 7.08 (s, 1H), 3.90 (s, 3H), 2.59 (s, 3H), 1.42 (s, 6H).

### Step 3: 3,3,5-Trimethyl-2-oxoindoline-6-carboxylic acid (149A-3)

To a solution of methyl 3,3,5-trimethyl-2-oxoindoline-6-carboxylate (100 mg, 429 μmol, 1.0 eq) in a mixture of MeOH (2.5 mL) and THF (7.5 mL) was added NaOH (2 M aqueous, 4.5 mL, 21 eq). The mixture was stirred at 70° C. for 2 h. LCMS indicated that the starting material was completely consumed, and the desired mass was detected. The reaction mixture was allowed to cool to room temperature, poured into H₂O (10 mL) and extracted with MTBE (3.0 mL×3). The aqueous layer was acidified to pH 5 using HCl (1 M aqueous). The product was extracted with 2-methyltetrahydrofuran (5.0 mL×3). The combined organic layers were dried over Na₂SO₄, filtered, and concentrated under vacuum to give the crude product 5-(2-((tert-butoxycarbonyl)amino)ethoxy)-2-methyl benzoic acid (90.0 mg), which was used in the next step without any further purification. M+H⁺=220.0 (LCMS); ¹H NMR (400 MHZ, DMSO-d₆) δ 12.88-12.61 (m, 1H), 10.38 (s, 1H), 7.27 (s, 1H), 7.23 (s, 1H), 2.47 (s, 3H), 1.25 (s, 6H).

### Step 4: 3,3,5-Trimethyl-N-(1-(naphthalen-1-yl)cyclopropyl)-2-oxoindoline-6-carboxamide (Compound 243)

To a solution of 1-(naphthalen-1-yl)cyclopropanamine (75.2 mg, 411 μmol, 1.0 eq) and 5-(2-((tert-butoxycarbonyl)amino)ethoxy)-2-methylbenzoic acid (90.0 mg, 411 μmol, 1.0 eq) in DCM (10 mL) were added TEA (125 mg, 1.23 mmol, 171 μL, 3.0 eq), EDCI (157 mg, 821 μmol, 2.0 eq) and HOBt (111 mg, 821 μmol, 2.0 eq). The mixture was stirred at 20° C. for 16 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 20%-50% B over 8 min; mobile phase A: 10 mM aqueous NH₄HCO₃, mobile phase B: acetonitrile). 3,3,5-Trimethyl-N-(1-(naphthalen-1-yl)cyclopropyl)-2-oxoindoline-6-carboxamide (45.3 mg, 117 μmol, 28% yield) was obtained as a white solid. M+H⁺=385.1 (LCMS); ¹H NMR (400 MHZ, DMSO-d₆) δ 10.22 (s, 1H), 9.12 (s, 1H), 8.62 (d, J=8.2 Hz, 1H), 7.93 (d, J=7.7 Hz, 1H), 7.82 (dd, J=7.8, 10.3 Hz, 2H), 7.61-7.42 (m, 3H), 7.05 (s, 1H), 6.47 (s, 1H), 1.97 (s, 3H), 1.32 (br s, 2H), 1.21-1.14 (m, 8H).

### Example 150: 3,3,5-Trimethyl-N-(1-(naphthalen-1-yl)cyclopropyl)indoline-6-carboxamide (Compound 262)

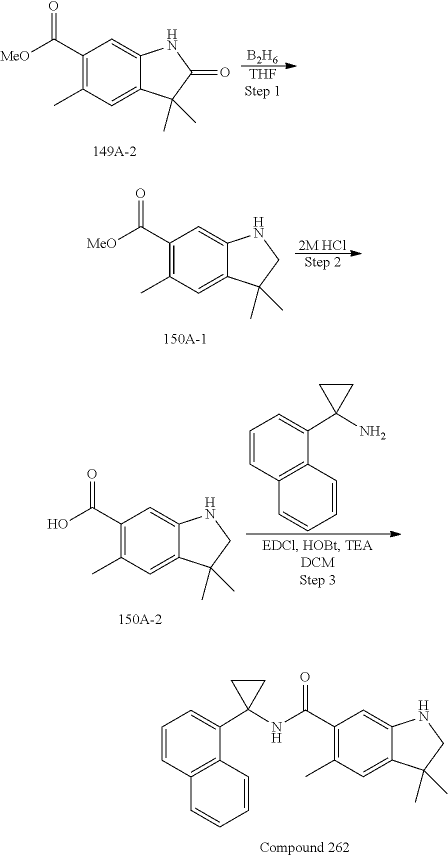

### Step 1: Methyl 3,3,5-trimethylindoline-6-carboxylate (150A-1)

To a solution of methyl 3,3,5-trimethyl-2-oxoindoline-6-carboxylate (200 mg, 857 μmol, 1.0 eq) in THF (50 mL) was slowly added BH₃·THF (1 M, 2.14 mL, 2.5 eq) at 0° C. The mixture was stirred at 25° C. for 16 h under a N₂ atmosphere. LCMS indicated that the starting material was completely consumed, and the desired mass was detected. MeOH (5.0 mL) was added and stirred at 70° C. for 1 h. The reaction mixture was allowed to cool to room temperature, poured into H₂O (20 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. Methyl 3,3,5-trimethylindoline-6-carboxylate (160 mg, 730 μmol, 85% yield) was obtained as a white solid. M+H⁺=220.1 (LCMS); ¹H NMR (400 MHZ, CDCl₃) δ 7.19 (s, 1H), 6.90 (s, 1H), 3.86 (s, 3H), 3.33 (s, 2H), 2.51 (s, 3H), 1.31 (s, 6H).

### Step 2: 3,3,5-Trimethylindoline-6-carboxylic acid (150A-2)

A solution of methyl 3,3,5-trimethylindoline-6-carboxylate (100 mg, 456 μmol, 1.0 eq) in HCl (2 M aqueous, 1 mL) was stirred at 100° C. for 1 h. LCMS indicated that the starting material was completely consumed, and the desired mass was detected. The reaction mixture was allowed to cool to room temperature, poured into H₂O (10 mL) and extracted with EtOAc (3.0 mL×3). The combined organic layers were dried over Na₂SO₄, filtered, and concentrated under vacuum to give the crude product 3,3,5-trimethylindoline-6-carboxylic acid (50.0 mg), which was used in the next step without any further purification. M+H⁺=206.1 (LCMS).

### Step 3: 3,3,5-Trimethyl-N-(1-(naphthalen-1-yl)cyclopropyl)indoline-6-carboxamide (Compound 262)

To a solution of 1-(naphthalen-1-yl)cyclopropanamine (44.6 mg, 244 μmol, 1.0 eq) and 3,3,5-trimethylindoline-6-carboxylic acid (50.0 mg, 244 μmol, 1.0 eq) in DCM (5.0 mL) were added TEA (49.3 mg, 487 μmol, 67.8 μL, 2.0 eq), EDCI (56.0 mg, 292 μmol, 1.2 eq) and HOBt (39.5 mg, 292 μmol, 1.2 eq). The mixture was stirred at 20° C. for 16 h. LCMS indicated that the starting material was completely consumed, and the desired mass was detected. The reaction mixture was poured into H₂O (5.0 mL) and extracted with DCM (3.0 mL×2). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 30%-70% B over 8 min; mobile phase A: 0.2% aqueous FA, mobile phase B: acetonitrile). 3,3,5-Trimethyl-N-(1-(naphthalen-1-yl)cyclopropyl)indoline-6-carboxamide (7.50 mg, 17.2 μmol, 7% yield) was obtained as a yellow solid. M+H⁺=371.2 (LCMS); ¹H NMR (400 MHZ, DMSO-d₆) δ 8.96 (s, 1H), 8.64 (d, J=8.1 Hz, 1H), 7.92 (d, J=7.6 Hz, 1H), 7.80 (dd, J=7.7, 9.9 Hz, 2H), 7.59-7.41 (m, 4H), 6.72 (s, 1H), 6.15 (s, 1H), 3.09 (s, 2H), 1.90 (s, 3H), 1.30 (br s, 2H), 1.15 (s, 8H).

### Example 151: 2-(Aminomethyl)-5-methyl-N-(1-(naphthalen-1-yl)cyclopropyl)indoline-6-carboxamide (Compound 190)

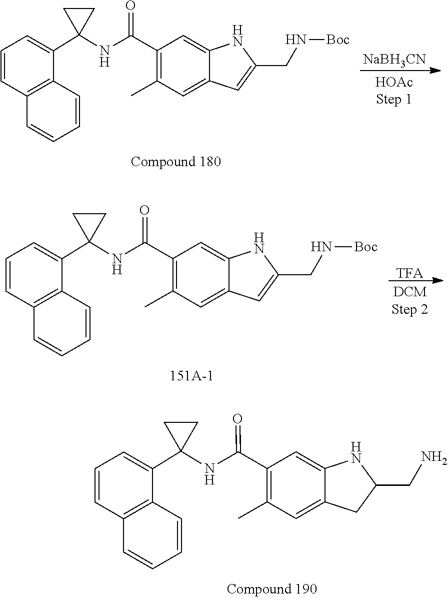

### Step 1: tert-Butyl((5-methyl-6-((1-(naphthalen-1-yl)cyclopropyl)carbamoyl) indolin-2-yl)methyl)carbamate (151A-1)

To a solution of tert-butyl((5-methyl-6-((1-(naphthalen-1-yl)cyclopropyl)carbamoyl)-1H-indol-2-yl)methyl)carbamate (225 mg, 479 μmol, 1.0 eq) in HOAc (15 mL) was added NaBH₃CN (60.2 mg, 958 μmol, 2.0 eq). The resulting mixture was stirred at 25° C. for 16 h. TLC indicated that that the starting material was completely consumed. The mixture was poured into H₂O (10 mL) and extracted with DCM (5.0 mL×5), and the combined organic layers were washed with saturated aqueous Na₂CO₃ solution (10 mL×2). The combined organic layers were dried over Na₂SO₄, filtered, and concentrated under vacuum to give a residue which was purified by flash silica gel chromatography using a gradient of EtOAc/petroleum ether from I/O to 1/1. tert-Butyl((5-methyl-6-((1-(naphthalen-1-yl)cyclopropyl)carbamoyl) indolin-2-yl)methyl)carbamate (115 mg, 243.86 μmol, 51% yield) was obtained as a white solid. ¹H NMR (400 MHZ, CDCl₃) δ 8.43 (d, J=8.5 Hz, 1H), 7.91 (dd, J=7.8, 15.6 Hz, 2H), 7.80 (d, J=8.3 Hz, 1H), 7.60-7.43 (m, 4H), 6.81 (s, 1H), 6.44 (s, 1H), 6.38 (s, 1H), 4.89-4.74 (m, 1H), 3.98 (br dd, J=2.4, 4.1 Hz, 1H), 3.27-3.15 (m, 1H), 3.04 (dd, J=8.9, 16.4 Hz, 1H), 2.67 (dd, J=7.6, 16.1 Hz, 1H), 2.07 (s, 3H), 1.54 (br d, J=1.8 Hz, 2H), 1.42 (s, 9H), 1.38 (br s, 2H).

### Step 2: 2-(Aminomethyl)-5-methyl-N-(1-(naphthalen-1-yl)cyclopropyl)indoline-6-carboxamide (Compound 190)

To a solution of tert-butyl((5-methyl-6-((1-(naphthalen-1-yl)cyclopropyl)carbamoyl) indolin-2-yl)methyl)carbamate (110 mg, 188 μmol, 1.0 eq) in DCM (7.0 mL) was added TFA (1.56 mL). The mixture was stirred at 25° C. for 10 min. LCMS indicated that the starting material was completely consumed, and the desired mass was detected. The reaction mixture was concentrated under vacuum to give a residue which was purified by preparative HPLC (Phenomenex Luna C18 column (80×30 mm, 3 μm); flow rate: 25 mL/min; gradient: 25%-55% B over 8 min; mobile phase A: 0.04% aqueous HCl, mobile phase B: acetonitrile). 2-(Aminomethyl)-5-methyl-N-(1-(naphthalen-1-yl)cyclopropyl)indoline-6-carboxamide (12.1 mg, 29.8 μmol, 16% yield, HCl salt) was obtained as a yellow solid. M+H⁺=372.3 (LCMS); ¹H NMR (400 MHZ, DMSO-d₆) δ 8.92 (s, 1H), 8.63 (d, J=8.3 Hz, 1H), 7.97-7.86 (m, 4H), 7.83-7.74 (m, 2H), 7.61-7.29 (m, 3H), 6.79 (s, 1H), 6.21 (s, 1H), 3.99-3.87 (m, 1H), 3.04-2.96 (m, 1H), 2.88-2.75 (m, 2H), 2.69 (br d, J=7.1 Hz, 1H), 1.89 (s, 3H), 1.30 (m, 2H), 1.18-1.09 (m, 2H).

### Example 152: 5-Methyl-2-((methylamino)methyl)-N-(1-(naphthalen-1-yl)cyclopropyl)indoline-6-carboxamide (Compound 231)

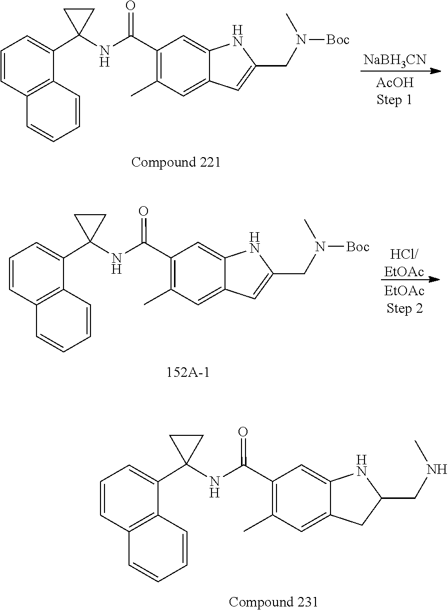

### Step 1: tert-Butyl methyl((5-methyl-6-((1-(naphthalen-1-yl)cyclopropyl)carbamoyl) indolin-2-yl)methyl)carbamate (152A-1)

To a solution of tert-butyl methyl((5-methyl-6-((1-(naphthalen-1-yl)cyclopropyl)carbamoyl)-1H-indol-2-yl)methyl)carbamate (90.0 mg, 186 μmol, 1.0 eq) in AcOH (5.0 mL) was added NaBH₃CN (23.4 mg, 372.21 μmol, 2.0 eq). The resulting mixture was stirred at 25° C. for 16 h. LCMS indicated that the starting material was completely consumed, and the desired mass was detected. The reaction mixture was quenched by the addition of saturated aqueous Na₂CO₃ (10 mL) and extracted with DCM (5.0 mL×3). The combined organic layers were dried over Na₂SO₄, filtered, and concentrated under vacuum to give crude product tert-butyl methyl((5-methyl-6-((1-(naphthalen-1-yl)cyclopropyl)carbamoyl) indolin-2-yl)methyl)carbamate (420 mg, 92% purity) as a yellow oil, which was used in the next step without any further purification. M+H⁺=486.2 (LCMS).

### Step 2: 5-Methyl-2-((methylamino)methyl)-N-(1-(naphthalen-1-yl)cyclopropyl)indoline-6-carboxamide (Compound 231)

To a solution of tert-butyl methyl((5-methyl-6-((1-(naphthalen-1-yl)cyclopropyl)carbamoyl) indolin-2-yl)methyl)carbamate (110 mg, 181 μmol, 1.0 eq) in EtOAc (5.0 mL) was added HCl/EtOAc (4 M, 2.4 mL), the mixture was stirred at 25° C. for 1 h. LCMS indicated that the starting material was completely consumed, and the desired mass was detected. The mixture was concentrated under vacuum at 30° C. to give a residue which was purified by preparative HPLC (Phenomenex Gemini C18 column (80×30 mm, 3 μm); flow rate: 25 mL/min; gradient: 1%-30% B over 8 min; mobile phase A: 0.04% aqueous HCl, mobile phase B: acetonitrile). 5-Methyl-2-((methylamino)methyl)-N-(1-(naphthalen-1-yl)cyclopropyl)indo line-6-carboxamide (15.4 mg, 36.0 μmol, 20% yield, HCl salt) was obtained as a yellow solid solid. M+H⁺=386.1 (LCMS); ¹H NMR (400 MHZ, DMSO-d₆) δ 8.97-8.90 (m, 1H), 8.65 (br d, J=8.5 Hz, 2H), 7.98-7.89 (m, 1H), 7.85-7.75 (m, 2H), 7.60-7.41 (m, 3H), 6.83-6.78 (m, 1H), 6.25-6.20 (m, 1H), 4.05-3.97 (m, 1H), 3.64-3.62 (m, 3H), 3.15-2.65 (m, 5H), 1.95-1.87 (m, 3H), 1.36-1.28 (m, 2H), 1.19-1.10 (m, 2H).

### Example 153: 5-Methyl-N-(1-(naphthalen-1-yl)cyclopropyl)-1H-indazole-6-carboxamide (Compound 176)

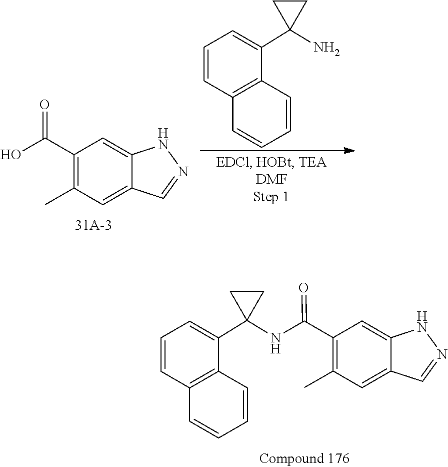

### Step 1: 5-Methyl-N-(1-(naphthalen-1-yl)cyclopropyl)-1H-indazole-6-carboxamide (Compound 176)

To a solution of 5-methyl-1H-indazole-6-carboxylic acid (50.0 mg, 284 μmol, 1.0 eq) and 1-(naphthalen-1-yl)cyclopropanamine (52.0 mg, 284 μmol, 1.0 eq) in DMF (5.0 mL) were added TEA (86.2 mg, 851 μmol, 119 μL, 3.0 eq), EDCI (81.6 mg, 426 μmol, 1.5 eq) and HOBt (57.5 mg, 426 μmol, 1.5 eq). The mixture was stirred at 20° C. for 16 h. LCMS indicated that the starting material was completely consumed, and the desired mass was detected. The reaction mixture was poured into H₂O (5.0 mL) at 25° C. and extracted with EtOAc (2.0 mL×3). The combined organic layers were washed with brine (2.0 mL×3), dried over Na₂SO₄, filtered, and concentrated under vacuum to give a residue which was purified by preparative HPLC (Phenomenex Luna C18 column (80×30 mm, 3 μm); flow rate: 25 mL/min; gradient: 15%-50% B over 8 min; mobile phase A: 0.04% aqueous HCl, mobile phase B: acetonitrile). 5-Methyl-N-(1-(naphthalen-1-yl)cyclopropyl)-1H-indazole-6-carboxamide (15.5 mg, 45.0 μmol, 16% yield, HCl salt) was obtained as a white solid. M+H⁺=342.1 (LCMS); ¹H NMR (400 MHz, DMSO-d₆) δ 9.24-9.19 (m, 1H), 8.18-8.14 (m, 1H), 7.98-7.91 (m, 2H), 7.88-7.80 (m, 2H), 7.61-7.45 (m, 5H), 6.64 (s, 1H), 2.12 (s, 3H), 1.42-1.35 (m, 2H), 1.22-1.15 (m, 2H).

### Example 154: 1-(Azetidin-3-yl)-5-methyl-N-(1-(naphthalen-1-yl)cyclopropyl)-1H-indazole-6-carboxamide (Compound 304) and 2-(azetidin-3-yl)-5-methyl-N-(1-(naphthalen-1-yl)cyclopropyl)-2H-indazole-6-carboxamide (Compound 305)

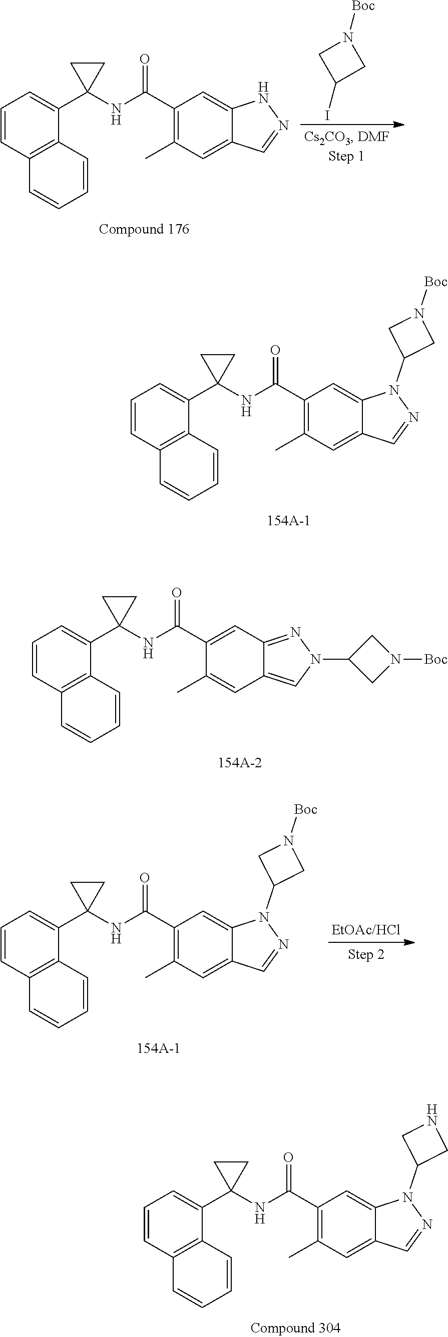

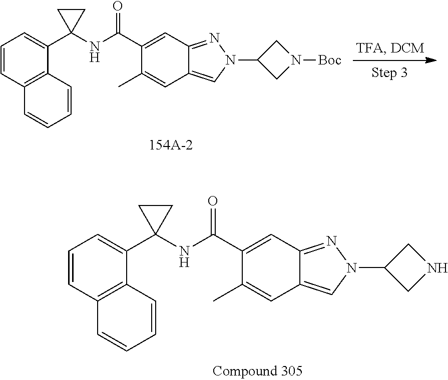 [SMILES: Cc1cc2cn(C3CN(C(=O)OC(C)(C)C)C3)nc2cc1C(=O)NC1(c2cccc3ccccc23)CC1 ; Cc1cc2cn(C3CNC3)nc2cc1C(=O)NC1(c2cccc3ccccc23)CC1]

### Step 1: tert-Butyl 3-(5-methyl-6-((1-(naphthalen-1-yl)cyclopropyl)carbamoyl)-1H-indazol-1-yl)azetidine-1-carboxylate (154A-1) and tert-butyl 3-(5-methyl-6-((1-(naphthalene-1-yl)cyclopropyl)carbamoyl)-2H-indazol-2-yl)azetidine-1-carboxylate (154A-2)

To a solution of 5-methyl-N-(1-(naphthalen-1-yl)cyclopropyl)-1H-indazole-6-carboxamide (150 mg, 439 μmol, 1.0 eq) and tert-butyl 3-iodoazetidine-1-carboxylate (249 mg, 879 μmol, 2.0 eq) in DMF (5.0 mL) was added cesium carbonate (429 mg, 1.32 mmol, 3.0 eq). The mixture was stirred at 100° C. for 16 h. LCMS indicated that the starting material was completely consumed, and the desired mass was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by preparative TLC (DCM/MeOH=10/1, R/1=0.4, R/2=0.6). tert-Butyl 3-(5-methyl-6-((1-(naphthalen-1-yl)cyclopropyl)carbamoyl)-1H-indazol-1-yl) azetidine-1-carboxylate (140 mg, 282 μmol, 64% yield) was obtained as a colorless oil. M+H⁺=497.2 (LCMS); ¹H NMR (400 MHZ, DMSO-d₆) δ 9.20 (s, 1H), 8.66 (d, J=8.4 Hz, 1H), 8.11 (s, 1H), 7.97-7.91 (m, 1H), 7.85 (dd, J=3.0, 7.6 Hz, 2H), 7.60-7.45 (m, 4H), 7.39 (s, 1H), 5.64-5.53 (m, 1H), 4.33-4.26 (m, 2H), 4.18 (br s, 2H), 2.05 (s, 3H), 1.41 (s, 11H), 1.23-1.19 (m, 2H). tert-Butyl 3-(5-methyl-6-((1-(naphthalen-1-yl)cyclopropyl)carbamoyl)-2H-indazol-2-yl)azetidine-1-carboxylate (50.0 mg, 101 μmol, 23% yield) was obtained as a colorless oil. M−56+H⁺=441.1 (LCMS); ¹H NMR (400 MHZ, DMSO-d₆) δ 9.21 (s, 1H), 8.69 (d, J=8.5 Hz, 1H), 8.38 (s, 1H), 7.95 (d, J=7.8 Hz, 1H), 7.87-7.80 (m, 2H), 7.63-7.57 (m, 1H), 7.56-7.51 (m, 1H), 7.47 (t, J=7.6 Hz, 1H), 7.40 (s, 1H), 7.33 (s, 1H), 5.54-5.40 (m, 1H), 4.36 (br t, J=8.2 Hz, 2H), 4.20 (br d, J=4.0 Hz, 2H), 2.10 (s, 3H), 1.42 (s, 9H), 1.38 (br s, 2H), 1.19 (s, 2H).

### Step 2: 1-(Azetidin-3-yl)-5-methyl-N-(1-(naphthalen-1-yl)cyclopropyl)-1H-indazole-6-carboxamide (Compound 304)

To a stirred solution of tert-butyl 3-(5-methyl-6-((1-(naphthalen-1-yl)cyclopropyl)carbamoyl)-1H-indazol-1-yl)azetidine-1-carboxylate (70.0 mg, 141 μmol, 1.0 eq) in EtOAc (2.0 mL) was added HCl/EtOAc (4 M, 6.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Gemini C18 column (80×30 mm, 3 μm); flow rate: 25 mL/min; gradient: 5%-35% B over 8 min; mobile phase A: 0.04% aqueous HCl, mobile phase B: acetonitrile). 1-(Azetidin-3-yl)-5-methyl-N-(1-(naphthalen-1-yl)cyclopropyl)-1H-indazole-6-carboxamide (22.3 mg, 50.9 μmol, 36% yield, HCl salt) was obtained as a white solid. M+H⁺=397.1 (LCMS); ¹H NMR (400 MHZ, DMSO-d₆) δ 9.38-9.23 (m, 2H), 9.20 (s, 1H), 8.66 (d, J=8.1 Hz, 1H), 8.21 (s, 1H), 7.97-7.92 (m, 1H), 7.86 (dd, J=4.1, 7.6 Hz, 2H), 7.62-7.44 (m, 5H), 5.78 (t, J=7.6 Hz, 1H), 4.45-4.31 (m, 4H), 2.07 (s, 3H), 1.46-1.36 (m, 2H), 1.27-1.16 (m, 2H).

### Step 3: 2-(Azetidin-3-yl)-5-methyl-N-(1-(naphthalen-1-yl)cyclopropyl)-2H-indazole-6-carboxamide (Compound 305)

To a stirred solution of tert-butyl 3-(5-methyl-6-((1-(naphthalen-1-yl)cyclopropyl)carbamoyl)-2H-indazol-2-yl)azetidine-1-carboxylate (70.0 mg, 141 μmol, 1.0 eq) in DCM (4.0 mL) was added TFA (800 μL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Gemini C18 column (150×30 mm, 5 μm); flow rate: 25 mL/min; gradient: 10%-40% B over 8 min; mobile phase A: 0.1% aqueous TFA, mobile phase B: acetonitrile). 2-(Azetidin-3-yl)-5-methyl-N-(1-(naphthalen-1-yl)cyclopropyl)-2H-indazole-6-carboxamide (20.9 mg, 40.9 μmol, 51% yield, TFA salt) was obtained as a white solid. M+H⁺=397.1 (LCMS); ¹H NMR (400 MHZ, DMSO-d₆) δ 9.23 (s, 1H), 9.15 (br d, J=10.1 Hz, 1H), 8.97-8.86 (m, 1H), 8.69 (d, J=8.5 Hz, 1H), 8.37 (s, 1H), 7.96 (d, J=7.9 Hz, 1H), 7.84 (t, J=8.1 Hz, 2H), 7.64-7.51 (m, 2H), 7.51-7.42 (m, 2H), 7.32 (s, 1H), 5.67 (quin, J=7.5 Hz, 1H), 4.54-4.34 (m, 4H), 2.10 (s, 3H), 1.39 (s, 2H), 1.26-1.17 (m, 2H).

### Example 155: 2-(Aminomethyl)-5-methyl-N-(1-(naphthalen-1-yl)cyclopropyl)benzofuran-6-carboxamide (Compound 197)

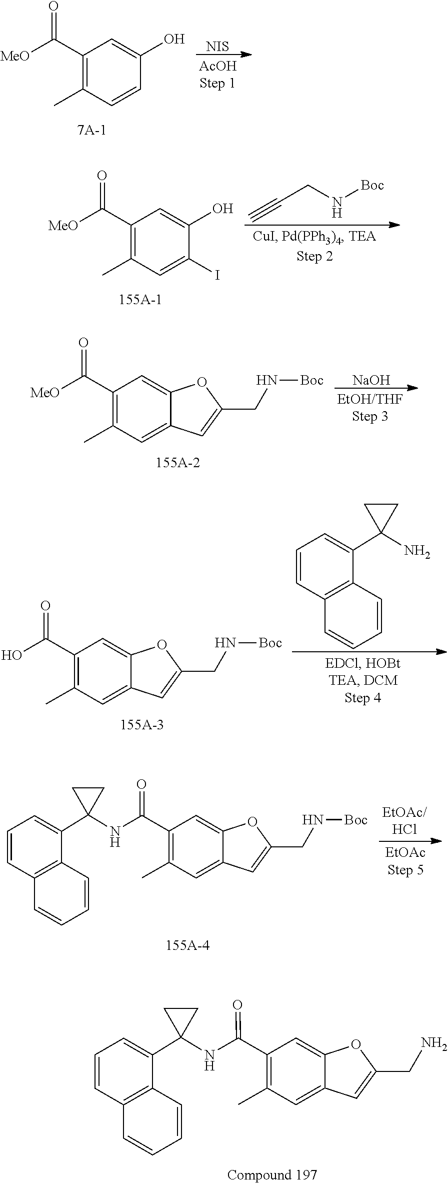

### Step 1: Methyl 5-hydroxy-4-iodo-2-methylbenzoate (155A-1)

To a solution of methyl 5-hydroxy-2-methylbenzoate (1.00 g, 6.02 mmol, 1.0 eq) in AcOH (10 mL) was added NIS (1.49 g, 6.62 mmol, 1.1 eq). The mixture was stirred at 20° C. for 16 h. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/10. Methyl 5-hydroxy-4-iodo-2-methylbenzoate (1.70 g, 5.82 mmol, 97% yield) was obtained as a white solid. M+H⁺=293.0 (LCMS).

### Step 2: Methyl 2-(((tert-butoxycarbonyl)amino)methyl)-5-methylbenzofuran-6-carboxylate (155A-2)

To a solution of methyl 5-hydroxy-4-iodo-2-methylbenzoate (1.70 g, 5.82 mmol, 1.0 eq) and tert-butyl prop-2-yn-1-ylcarbamate (903 mg, 5.82 mmol, 1.0 eq) in a mixture of H₂O (10 mL) and toluene (20 mL) were added TEA (1.18 g, 11.6 mmol, 1.62 mL, 2.0 eq), Pd(PPh₃)₂Cl₂ (123 mg, 175 μmol, 0.03 eq) and CuI (111 mg, 582 μmol, 0.1 eq). The mixture was stirred at 70° C. for 3 h. LCMS indicated that the starting material was completely consumed, and the desired mass was detected. The reaction mixture was allowed to cool to room temperature, poured into H₂O (30 mL) and extracted with EtOAc (20 mL×2). The combined organic layers were dried over Na₂SO₄, filtered, and concentrated under vacuum to give a residue which was purified by flash silica gel chromatography using a gradient of EtOAc/petroleum ether from 0/1 to 1/7. Methyl 2-(((tert-butoxycarbonyl)amino)methyl)-5-methylbenzofuran-6-carboxylate (1.00 g, 3.13 mmol, 54% yield) was obtained as a yellow oil. M−56+H⁺=264.1 (LCMS).

### Step 3: 2-(((tert-Butoxycarbonyl)amino)methyl)-5-methylbenzofuran-6-carboxylic acid (155A-3)

To a solution of methyl 2-(((tert-butoxycarbonyl)amino)methyl)-5-methylbenzofuran-6-carboxylate (800 mg, 2.51 mmol, 1.0 eq) in a mixture of THF (5.0 mL) and EtOH (5.0 mL) was added NaOH (2 M in aqueous, 3.2 mL). The mixture was stirred at 80° C. for 2 h. LCMS indicated that the starting material was completely consumed, and the desired mass was detected. The reaction mixture was allowed to cool to room temperature, poured into H₂O (10 mL) and extracted with MTBE (2.0 mL×3). The aqueous layer was basified to pH 4 using HCl (1 M aqueous) and extracted with EtOAc (8.0 mL×2). The combined organic layers were dried over Na₂SO₄, filtered, and concentrated under vacuum to give the crude product 2-(((tert-butoxycarbonyl)amino)methyl)-5-methylbenzofuran-6-carboxylic acid (700 mg) as a white solid. M−56+H⁺=250.1 (LCMS).

### Step 4: tert-Butyl((5-methyl-6-((1-(naphthalen-1-yl)cyclopropyl)carbamoyl)benzofuran-2-yl)methyl)carbamate (155A-4)

To a solution of 2-(((tert-butoxycarbonyl)amino)methyl)-5-methylbenzofuran-6-carboxylic acid (700 mg, 2.29 mmol, 1.0 eq) and 1-(naphthalen-1-yl)cyclopropanamine (441 mg, 2.41 mmol, 20.0 μL, 1.0 eq) in DCM (10 mL) were added TEA (696 mg, 6.88 mmol, 957 μL, 3.0 eq), EDCI (879 mg, 4.59 mmol, 2.0 eq) and HOBt (620 mg, 4.59 mmol, 2.0 eq). The mixture was stirred at 20° C. for 3 h. LCMS indicated that the starting material was completely consumed, and the desired mass was detected. The reaction mixture was poured into H₂O (10 mL) and extracted with DCM (10 mL×2). The combined organic layers were dried over Na₂SO₄, filtered, and concentrated under vacuum to give a residue which was purified by flash silica gel chromatography using a gradient of EtOAc/petroleum ether from 0/1 to 1/5. tert-Butyl((5-methyl-6-((1-(naphthalen-1-yl)cyclopropyl)carbamoyl)benzofuran-2-yl)methyl)carbamate (800 mg, 1.70 mmol, 74% yield) was obtained as a white solid. M+H⁺=471.3 (LCMS); ¹H NMR (400 MHz, CDCl₃) δ 8.42-8.36 (m, 1H), 7.91-7.79 (m, 2H), 7.76-7.70 (m, 1H), 7.54-7.37 (m, 3H), 7.16-7.12 (m, 2H), 6.47-6.36 (m, 2H), 4.87-4.79 (m, 1H), 4.46-4.22 (m, 2H), 2.22-2.15 (m, 3H), 1.41-1.39 (m, 2H), 1.35 (br s, 9H), 1.35-1.31 (m, 2H).

### Step 5: 2-(Aminomethyl)-5-methyl-N-(1-(naphthalen-1-yl)cyclopropyl)benzofuran-6-carboxamide (Compound 197)

To a solution of tert-butyl((5-methyl-6-((1-(naphthalen-1-yl)cyclopropyl)carbamoyl)benzofuran-2-yl)methyl)carbamate (80.0 mg, 640 μmol, 1.0 eq) in EtOAc (3.0 mL) was added HCl/EtOAc (4 M, 1.0 mL). The mixture was stirred at 20° C. for 1 h. LCMS indicated that the starting material completely consumed, and the desired mass was detected. The mixture was concentrated under vacuum to give a residue which was purified by preparative HPLC (Phenomenex Luna C18 column (80×30 mm, 3 μm); flow rate: 25 mL/min; gradient: 15%-45% B over 8 min; mobile phase A: 0.04% aqueous HCl, mobile phase B: acetonitrile). 2-(Aminomethyl)-5-methyl-N-(1-(naphthalen-1-yl)cyclopropyl)benzofuran-6-carboxamide (26.6 mg, 65.5 μmol, 19% yield, HCl salt) was obtained as a white solid. M+H⁺=371.1 (LCMS); ¹H NMR (400 MHZ, DMSO-d₆) δ 9.18 (s, 1H), 8.67 (d, J=8.3 Hz, 1H), 8.54 (br s, 2H), 7.97-7.91 (m, 1H), 7.83 (s, 2H), 7.62-7.44 (m, 3H), 7.43-7.41 (m, 1H), 7.27-7.22 (m, 1H), 6.96-6.90 (m, 1H), 4.40-4.16 (m, 2H), 2.13 (s, 3H), 1.60-1.31 (m, 2H), 1.27-1.06 (m, 2H).

### Example 156: 2-((Dimethylamino)methyl)-5-methyl-N-(1-(naphthalen-1-yl)cyclopropyl)benzofuran-6-carboxamide (Compound 198)

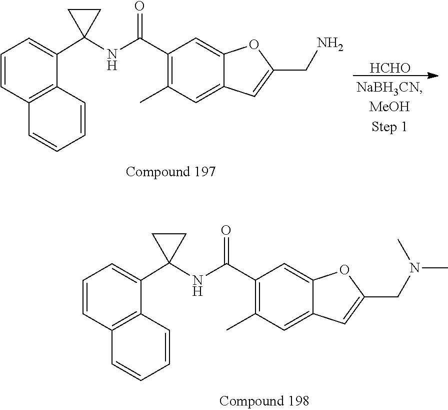

### Step 1: 2-((Dimethylamino)methyl)-5-methyl-N-(1-(naphthalen-1-yl)cyclopropyl)benzofuran-6-carboxamide (Compound 198)

To a solution of 2-(aminomethyl)-5-methyl-N-(1-(naphthalen-1-yl)cyclopropyl)benzofuran-6-carboxamide (70.0 mg, 189 μmol, 1.0 eq, HCl salt) in MeOH (2.0 mL) was added TEA (10.0 μL), followed by the addition of formaldehyde (15.3 mg, 189 μmol, 14.1 μL, 37% purity, 2.0 eq). The resulting mixture was adjusted to pH 6 with a small amount of AcOH. The mixture was stirred at 20° C. for 30 min, then NaBH₃CN (11.9 mg, 189 μmol, 1.0 eq) was added. The reaction mixture was stirred at 20° C. for another 1 h. LCMS indicated that the starting material was completely consumed, and the desired mass was detected. The reaction mixture was poured into H₂O (5.0 mL) and extracted with EtOAc (3.0 mL×2). The combined organic layers were dried over Na₂SO₄, filtered, and concentrated under vacuum to give a residue which was purified by preparative HPLC (Phenomenex Luma C18 column (80×30 mm, 3 μm); flow rate: 25 mL/min; gradient: 1%-45% B over 8 min; mobile phase A: 0.04% aqueous HCl, mobile phase B: acetonitrile). 2-((Dimethylamino)methyl)-5-methyl-N-(1-(naphthalen-1-yl)cyclopropyl)-2,3-dihydrobenzofuran-6-carboxamide (30.9 mg, 77.5 μmol, 41% yield, HCl salt) was obtained as a white solid. M+H⁺=399.1 (LCMS); ¹H NMR (400 MHZ, DMSO-d₆) δ 9.19 (s, 1H), 8.70-8.61 (m, 1H), 7.93 (d, J=8.0 Hz, 1H), 7.87-7.78 (m, 2H), 7.63-7.43 (m, 4H), 7.30-7.26 (m, 1H), 7.13-7.09 (m, 1H), 4.51 (br s, 2H), 2.74 (br s, 6H), 2.11 (s, 3H), 1.42-1.33 (m, 2H), 1.24-1.14 (m, 2H).

### Example 157: 5-Methyl-2-((methylamino)methyl)-N-(1-(naphthalen-1-yl)cyclopropyl)benzofuran-6-carboxamide (Compound 220)

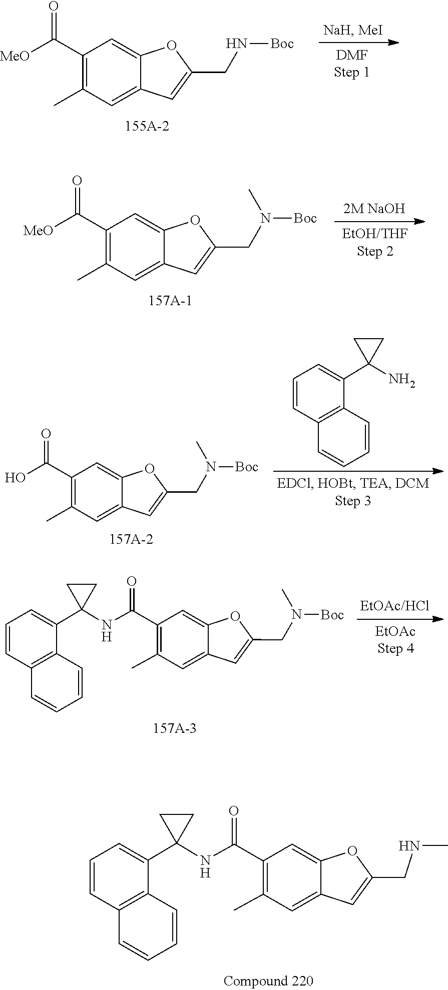 [SMILES: COC(=O)c1cc2oc(CNC(=O)OC(C)(C)C)cc2cc1C ; COC(=O)c1cc2oc(CN(C)C(=O)OC(C)(C)C)cc2cc1C ; Cc1cc2cc(CN(C)C(=O)OC(C)(C)C)oc2cc1C(=O)O ; NC1(c2cccc3ccccc23)CC1 ; Cc1cc2cc(CN(C)C(=O)OC(C)(C)C)oc2cc1C(=O)NC1(c2cccc3ccccc23)CC1 ; CNCc1cc2cc(C)c(C(=O)NC3(c4cccc5ccccc45)CC3)cc2o1]

### Step 1: Methyl 2-(((tert-butoxycarbonyl)(methyl)amino)methyl)-5-methylbenzofuran-6-carboxylate (157A-1)

To a solution of methyl 2-(((tert-butoxycarbonyl)amino)methyl)-5-methylbenzofuran-6-carboxylate (200 mg, 626 μmol, 1.0 eq) in DMF (5.0 mL) was degassed and purged with N₂ three times. To the mixture was added sodium hydride (50.1 mg, 1.25 mmol, 60% purity, 2.0 eq) dropwise at 0° C. for 30 min, then iodomethane (88.9 mg, 626 μmol, 39.0 μL, 1.0 eq) was added and the mixture was stirred at 0° C. for 2 h under a N₂ atmosphere. LCMS indicated that the starting material was completely consumed, and the desired mass was detected. The reaction mixture was poured into ice water (10 mL) and extracted with EtOAc (3.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. Methyl 2-(((tert-butoxycarbonyl)(methyl)amino)methyl)-5-methylbenzofuran-6-carboxylate (220 mg, 660 μmol, 53% yield) was obtained as a colorless oil. M−56+H⁺=278.1 (LCMS); ¹H NMR (400 MHZ, CDCl₃) δ 8.05 (s, 1H), 7.37 (s, 1H), 6.61-6.44 (m, 1H), 4.61-4.47 (m, 2H), 3.92 (s, 3H), 3.01-2.91 (m, 3H), 2.70-2.64 (m, 3H), 1.52-1.46 (m, 9H).

### Step 2: 2-(((tert-Butoxycarbonyl)(methyl)amino)methyl)-5-methylbenzofuran-6-carboxylic acid (157A-2)

To a solution of methyl 2-(((tert-butoxycarbonyl)(methyl)amino)methyl)-5-methylbenzofuran-6-carboxylate (200 mg, 600 μmol, 1.0 eq) in a mixture of MeOH (5.0 mL) and THF (15 mL) was added NaOH (2 M aqueous, 6.3 mL, 21 eq). The mixture was stirred at 25° C. for 1 h. Then the mixture was stirred at 70° C. for 16 h. LCMS indicated that the starting material was completely consumed, and the desired mass was detected. The reaction mixture was allowed to cool to room temperature, H₂O (20 mL) was added, and the mixture was washed with MTBE (5.0 mL×3). The aqueous layer was acidified to pH 5 using HCl (1 M aqueous). The product was extracted with 2-methyltetrahydrofuran (5.0 mL×3). The combined organic layers were dried over Na₂SO₄, filtered, and concentrated under vacuum to give the crude product 5-(2-((tert-butoxycarbonyl)amino)ethoxy)-2-methyl benzoic acid (180 mg), which was used in the next step without any further purification. M+H⁺=320.1 (LCMS); ¹H NMR (400 MHZ, CDCl₃) δ 8.20 (s, 1H), 7.40 (s, 1H), 6.61-6.49 (m, 1H), 4.56 (br d, J=14.8 Hz, 2H), 2.98 (br s, 3H), 2.73 (s, 3H), 1.54-1.46 (m, 9H).

### Step 3: tert-Butyl methyl((5-methyl-6-((1-(naphthalen-1-yl)cyclopropyl)carbamoyl)benzofuran-2-yl)methyl)carbamate (157A-3)

To a solution of 1-(naphthalen-1-yl)cyclopropanamine (100 mg, 546 μmol, 1.0 eq) and 2-(((tert-butoxycarbonyl)(methyl)amino)methyl)-5-methylbenzofuran-6-carboxylic acid (174 mg, 546 μmol, 1.0 eq) in DCM (10 mL) were added TEA (166 mg, 1.64 mmol, 228 μL, 3.0 eq), EDCI (209 mg, 1.09 mmol, 2.0 eq) and HOBt (148 mg, 1.09 mmol, 2.0 eq). The mixture was stirred at 20° C. for 16 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. tert-Butyl methyl((5-methyl-6-((1-(naphthalen-1-yl)cyclopropyl)carbamoyl)benzofuran-2-yl)methyl)carbamate (150 mg, 311 μmol, 57% yield) was obtained as a white solid. M+H⁺=485.2 (LCMS); ¹H NMR (400 MHZ, CDCl₃) δ 8.47 (d, J=8.4 Hz, 1H), 8.00-7.88 (m, 2H), 7.81 (d, J=8.2 Hz, 1H), 7.64-7.45 (m, 3H), 7.23 (s, 2H), 6.55-6.38 (m, 2H), 4.53-4.40 (m, 2H), 2.97-2.84 (m, 3H), 2.27 (s, 3H), 1.46 (br s, 9H), 0.91-0.83 (m, 4H).

### Step 4: 5-Methyl-2-((methylamino)methyl)-N-(1-(naphthalen-1-yl)cyclopropyl)benzofuran-6-carboxamide (Compound 220)

To a stirred solution of tert-butyl methyl((5-methyl-6-((1-(naphthalen-1-yl)cyclopropyl) carbamoyl)benzofuran-2-yl)methyl)carbamate (50.0 mg, 103 μmol, 1.0 eq) in EtOAc (1.0 mL) was added HCl/EtOAc (4 M, 8.0 mL). The mixture was stirred at 25° C. for 1 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 5%-40% B over 8 min; mobile phase A: 0.04% aqueous HCl, mobile phase B: acetonitrile). 5-Methyl-2-((methylamino)methyl)-N-(1-(naphthalen-1-yl)cyclopropyl)benzofuran-6-carboxamide (9.04 mg, 21.5 μmol, 21% yield, HCl salt) was obtained as a white solid. M+H⁺=385.1 (LCMS); ¹H NMR (400 MHZ, DMSO-d₆) δ 9.20 (s, 1H), 8.67 (d, J=8.2 Hz, 1H), 7.97-7.92 (m, 1H), 7.84 (t, J=7.6 Hz, 2H), 7.62-7.44 (m, 4H), 7.26 (s, 1H), 7.01 (s, 1H), 4.36 (s, 2H), 2.57 (s, 3H), 2.13 (s, 3H), 1.39 (s, 2H), 1.25-1.17 (m, 2H).

### Example 158: (S)-2-(Aminomethyl)-5-methyl-N-(1-(naphthalen-1-yl)cyclopropyl)-2,3-dihydrobenzofuran-6-carboxamide (Compound 235) and (R)-2-(Aminomethyl)-5-methyl-N-(1-(naphthalen-1-yl)cyclopropyl)-2,3-dihydrobenzofuran-6-carboxamide (Compound 236)

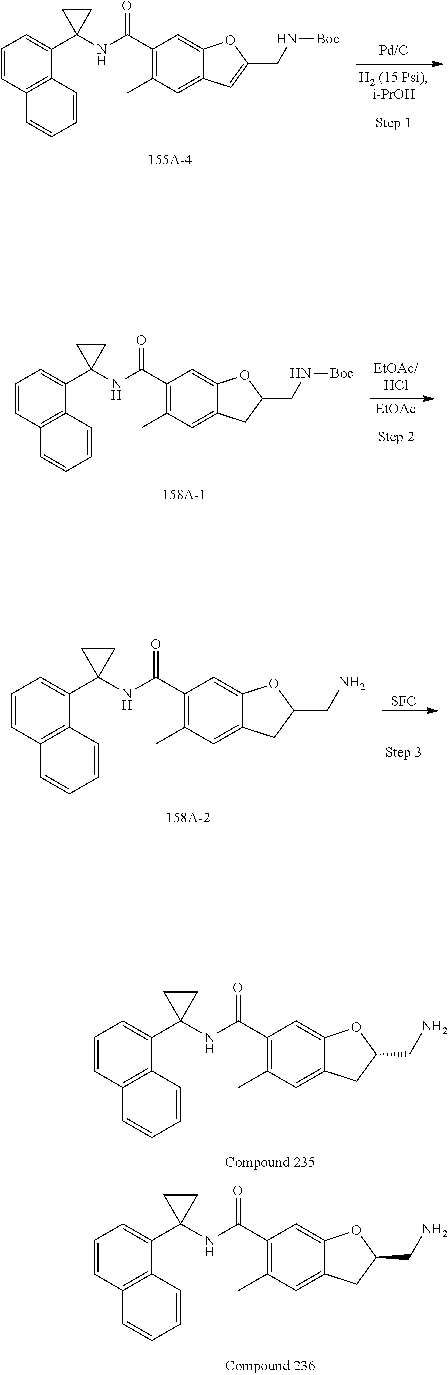

### Step 1: tert-Butyl((5-methyl-6-((1-(naphthalen-1-yl)cyclopropyl)carbamoyl)-2,3-dihydrobenzofuran-2-yl)methyl)carbamate (158A-1)

To a solution of tert-butyl((5-methyl-6-((1-(naphthalen-1-yl)cyclopropyl)carbamoyl)nbenzo furan-2-yl)methyl)carbamate (200 mg, 425 μmol, 1.0 eq) in i-PrOH (20 mL) was added 10% palladium on carbon (100 mg) under a N₂ atmosphere. The suspension was degassed and purged with H₂ for three times. The mixture was stirred at 20° C. for 16 h under a H₂ (15 psi) atmosphere. LCMS indicated that the starting material completely consumed, and the desired product was detected. The suspension was filtered through a pad of Celite, and the filter cake was washed with i-PrOH (4.0 mL×5). The combined filtrates were concentrated under vacuum to give the crude product tert-butyl((5-methyl-6-((1-(naphthalen-1-yl)cyclopropyl) carbamoyl)-2,3-dihydrobenzofuran-2-yl)methyl)carbamate (400 mg) as a colorless oil. M−56+H⁺=417.2 (LCMS).

### Step 2: 2-(Aminomethyl)-5-methyl-N-(1-(naphthalen-1-yl)cyclopropyl)-2,3-dihydro benzofuran-6-carboxamide (158A-2)

To a solution of tert-butyl((5-methyl-6-((1-(naphthalen-1-yl)cyclopropyl)carbamoyl)-2,3-dihydrobenzofuran-2-yl)methyl)carbamate (400 mg, 846 μmol, 1.0 eq) in EtOAc (10 mL) was added HCl/EtOAc (4 M, 498 μL). The mixture was stirred at 20° C. for 2 h. LCMS indicated that the starting material completely consumed, and the desired product was detected. The mixture was concentrated under vacuum to give a residue which was purified by preparative HPLC (Phenomenex Luna column (80×30 mm, 3 μm); flow rate: 25 mL/min; gradient: 1%-35% B over 8 min; mobile phase A: 0.04% aqueous HCl, mobile phase B: acetonitrile). 2-(Aminomethyl)-5-methyl-N-(1-(naphthalen-1-yl)cyclopropyl)-2,3-dihydrobenzofuran-6-carboxamide (120 mg, 293 μmol, 17% yield, HCl salt) was obtained as a white solid. M+H⁺=373.3 (LCMS).

### Step 3: (S)-2-(Aminomethyl)-5-methyl-N-(1-(naphthalen-1-yl)cyclopropyl)-2,3-dihydrobenzofuran-6-carboxamide (Compound 235) and (R)-2-(Aminomethyl)-5-methyl-N-(1-(naphthalen-1-yl)cyclopropyl)-2,3-dihydrobenzofuran-6-carboxamide (Compound 236)

2-(Aminomethyl)-5-methyl-N-(1-(naphthalen-1-yl)cyclopropyl)-2,3-dihydrobenzofuran-6-carboxamide (120 mg, 293 μmol, HCl salt) was further separated by SFC (DAICEL CHIRALPAK AD column (250×30 mm, 10 μm); flow rate: 3.4 mL/min; gradient: 42%-42% B over 8 min; mobile phase A: CO₂, mobile phase B: 0.1% isopropyl amine in EtOH). (R)-2-(Aminomethyl)-5-methyl-N-(1-(naphthalen-1-yl)cyclopropyl)-2,3-dihydrobenzofuran-6-carb oxamide (17.8 mg) was obtained as a white solid. M+H⁺=373.1 (LCMS); ¹H NMR (400 MHZ, CDCl₃) δ 8.47-8.42 (m, 1H), 7.95-7.85 (m, 2H), 7.82-7.76 (m, 1H), 7.64-7.40 (m, 3H), 6.87 (s, 1H), 6.53 (s, 1H), 4.88-4.65 (m, 1H), 3.17 (br dd, J=9.2, 16.1 Hz, 1H), 3.00-2.71 (m, 3H), 2.09 (s, 3H), 1.57-1.51 (m, 2H), 1.42-1.34 (m, 2H). (S)-2-(Aminomethyl)-5-methyl-N-(1-(naphthalen-1-yl)cyclopropyl)-2,3-dihydrobenzofuran-6-carboxamide (15.4 mg) was obtained as a white solid. M+H⁺=373.1 (LCMS); ¹H NMR (400 MHz, CDCl₃) δ 8.47-8.42 (m, 1H), 7.95-7.85 (m, 2H), 7.82-7.76 (m, 1H), 7.64-7.40 (m, 3H), 6.87 (s, 1H), 6.53 (s, 2H), 4.88-4.65 (m, 1H), 3.17 (br dd, J=9.2, 16.1 Hz, 1H), 3.00-2.71 (m, 3H), 2.09 (s, 3H), 1.57-1.51 (m, 2H), 1.42-1.34 (m, 2H).

### Example 159: (S)-2-Methyl-5-((1-methylazetidin-2-yl)methoxy)-N-(1-(quinolin-5-yl)cyclopropyl)benzamide (Compound 230)

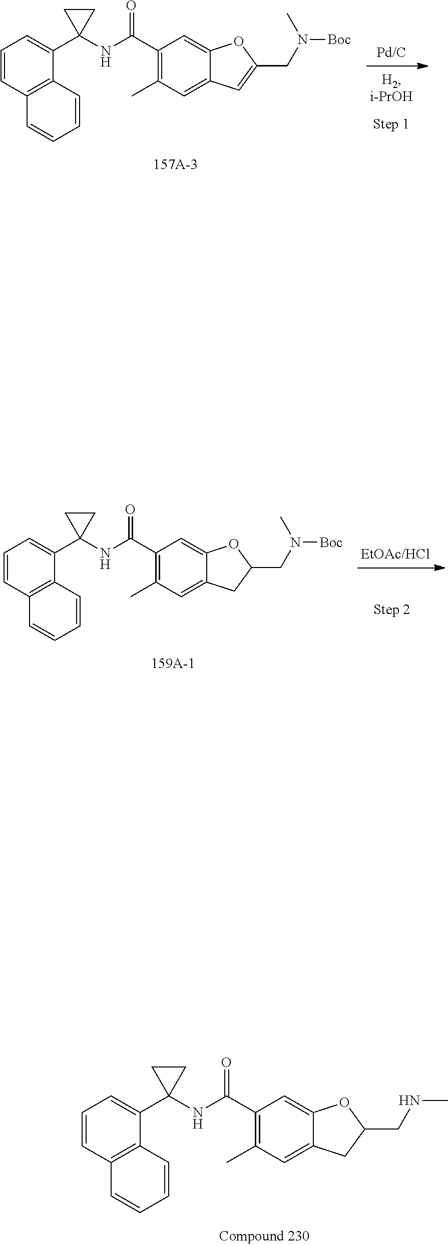 [SMILES: Cc1cc2cc(CN(C)C(=O)OC(C)(C)C)oc2cc1C(=O)NC1(c2cccc3ccccc23)CC1 ; Cc1cc2c(cc1C(=O)NC1(c3cccc4ccccc34)CC1)OC(CN(C)C(=O)OC(C)(C)C)C2 ; CNCC1Cc2cc(C)c(C(=O)NC3(c4cccc5ccccc45)CC3)cc2O1]

### Step 1: tert-Butyl methyl((5-methyl-6-((1-(naphthalen-1-yl)cyclopropyl)carbamoyl)-2,3-dihydrobenzofuran-2-yl)methyl)carbamate (159A-1)

To a solution of tert-butyl methyl((5-methyl-6-((1-(naphthalen-1-yl)cyclopropyl)carbamoyl)benzofuran-2-yl)methyl)carbamate (60.0 mg, 61.9 μmol, 1.0 eq) in i-PrOH (20 mL) was added 10% palladium on carbon (60.0 mg). The mixture was degassed and purged with H₂ three times and then the mixture was stirred at 25° C. for 4 h under a H₂ atmosphere. LCMS indicated that 40% starting material still remained and 50% desired mass was detected. The combined organic layers were filtered through a pad of Celite and concentrated under vacuum to give a residue which was purified by preparative TLC (EtOAc/petroleum ether=1/3, R_{f}=0.3). tert-Butylmethyl((5-methyl-6-((1-(naphthalen-1-yl)cyclopropyl)carbamoyl)-2,3-dihydro benzofuran-2-yl)methyl)carbamate (40.0 mg, 41.1 μmol, 33% yield) was obtained as a yellow oil. M−56+H⁺=431.2 (LCMS); ¹H NMR (400 MHZ, CDCl₃) δ 8.44 (d, J=8.4 Hz, 1H), 7.92 (dd, J=7.6, 15.7 Hz, 2H), 7.80 (d, J=7.5 Hz, 1H), 7.61-7.44 (m, 3H), 6.90 (s, 1H), 6.54 (s, 1H), 6.43 (s, 1H), 4.99-4.84 (m, 1H), 3.67-3.48 (m, 1H), 3.22-3.11 (m, 1H), 2.93-2.88 (m, 3H), 2.85 (d, J=7.2 Hz, 1H), 2.81 (d, J=7.2 Hz, 1H), 2.10 (s, 3H), 1.44 (br s, 9H), 1.41-1.37 (m, 2H), 1.30-1.25 (m, 2H).

### Step 2: 5-Methyl-2-((methylamino)methyl)-N-(1-(naphthalen-1-yl)cyclopropyl)-2,3-dihydrobenzofuran-6-carboxamide (Compound 230)

To a stirred solution of tert-butyl methyl((5-methyl-6-((1-(naphthalen-1-yl)cyclopropyl) carbamoyl)-2,3-dihydrobenzofuran-2-yl)methyl)carbamate (30.0 mg, 61.7 μmol, 1.0 eq) in EtOAc (1.0 mL) was added HCl/EtOAc (4 M, 2.0 mL). The mixture was stirred at 25° C. for 1 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 5%-30% B over 8 min; mobile phase A: 0.04% aqueous HCl, mobile phase B: acetonitrile). 5-Methyl-2-((methylamino)methyl)-N-(1-(naphthalen-1-yl)cyclopropyl)-2,3-dihydrobenzofuran-6-carboxamide (8.20 mg, 19.0 μmol, 31% yield, HCl salt) was obtained as a white solid. M+H⁺=387.0 (LCMS); ¹H NMR (400 MHZ, DMSO-d₆) δ 9.06 (s, 2H), 8.89-8.75 (m, 1H), 8.64 (d, J=8.3 Hz, 1H), 7.93 (d, J=7.7 Hz, 1H), 7.81 (dd, J=7.6, 13.6 Hz, 2H), 7.62-7.49 (m, 2H), 7.48-7.43 (m, 1H), 7.01 (s, 1H), 6.44 (s, 1H), 5.14-4.96 (m, 1H), 3.30 (dd, J=9.4, 16.6 Hz, 1H), 3.23-3.14 (m, 1H), 3.08 (ddd, J=4.6, 8.5, 12.7 Hz, 1H), 2.90 (dd, J=6.5, 16.2 Hz, 1H), 2.56 (br t, J=5.3 Hz, 3H), 1.98 (s, 3H), 1.33 (s, 2H), 1.24-1.10 (m, 2H).

### Example 160: 2-((Dimethylamino)methyl)-5-methyl-N-(1-(naphthalen-1-yl)cyclopropyl)-2,3-dihydrobenzofuran-6-carboxamide (Compound 224)

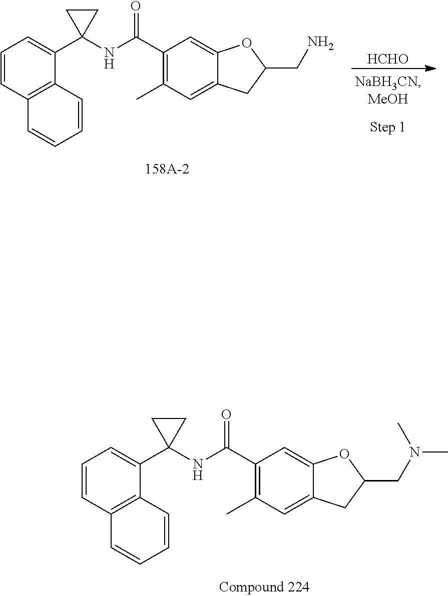 [SMILES: Cc1cc2c(cc1C(=O)NC1(c3cccc4ccccc34)CC1)OC(CN)C2 ; "HCHO" ; Cc1cc2c(cc1C(=O)NC1(c3cccc4ccccc34)CC1)OC(CN(C)C)C2]

### Step 1: 2-((Dimethylamino)methyl)-5-methyl-N-(1-(naphthalen-1-yl)cyclopropyl)-2,3-dihydrobenzofuran-6-carboxamide (Compound 224)

To a solution of 2-(aminomethyl)-5-methyl-N-(1-(naphthalen-1-yl)cyclopropyl)-2,3-dihydrobenzofuran-6-carboxamide (101 mg, 270 μmol, 1.0 eq, HCl salt) in MeOH (3.0 mL) was added TEA (50 μL), followed by the addition of formaldehyde (46.7 mg, 540 mmol, 59.1 μL, 37% purity in water, 2.0 eq). The resulting mixture was adjusted to pH 6 with a small amount of AcOH. The mixture was stirred at 20° C. for 30 min, then NaBH₃CN (33.9 mg, 540 μmol, 2.0 eq) was added. The reaction mixture was stirred at 20° C. for another 3 h. LCMS indicated that the starting material was completely consumed, and the desired mass was detected. The reaction mixture was poured into H₂O (5.0 mL) and extracted with EtOAc (3.0 mL×2). The combined organic layers were dried over Na₂SO₄, filtered, and concentrated under vacuum to give a residue which was purified by preparative HPLC (Phenomenex Luma C18 column (80×30 mm, 3 μm); flow rate: 25 mL/min; gradient: 10%-35% B over 8 min; mobile phase A: 0.04% aqueous HCl, mobile phase B: acetonitrile). 2-((Dimethylamino)methyl)-5-methyl-N-(1-(naphthalen-1-yl)cyclopropyl)-2,3-dihydrobenzo furan-6-carboxamide (17.5 mg, 42.8 μmol, 16% yield HCl salt) was obtained as a white solid. M+H⁺=401.1 (LCMS); 1H NMR (400 MHZ, CDCl₃) δ 12.80-12.43 (m, 1H), 8.55-8.42 (m, 1H), 7.90 (br dd, J=7.7, 11.2 Hz, 2H), 7.79 (br d, J=8.1 Hz, 1H), 7.62-7.41 (m, 3H), 6.90-6.80 (m, 1H), 6.57-6.47 (m, 1H), 5.40 (br s, 1H), 3.47-3.24 (m, 2H), 3.19-3.05 (m, 1H), 2.98-2.83 (m, 6H), 2.79-2.68 (m, 1H), 2.11-1.97 (m, 3H), 1.63-1.47 (m, 2H), 1.44-1.30 (m, 2H).

### Example 161: 5-Methyl-N-(1-(naphthalen-1-yl)cyclopropyl)-3-propyl-2,3-dihydrobenzofuran-6-carboxamide (Compound 397)

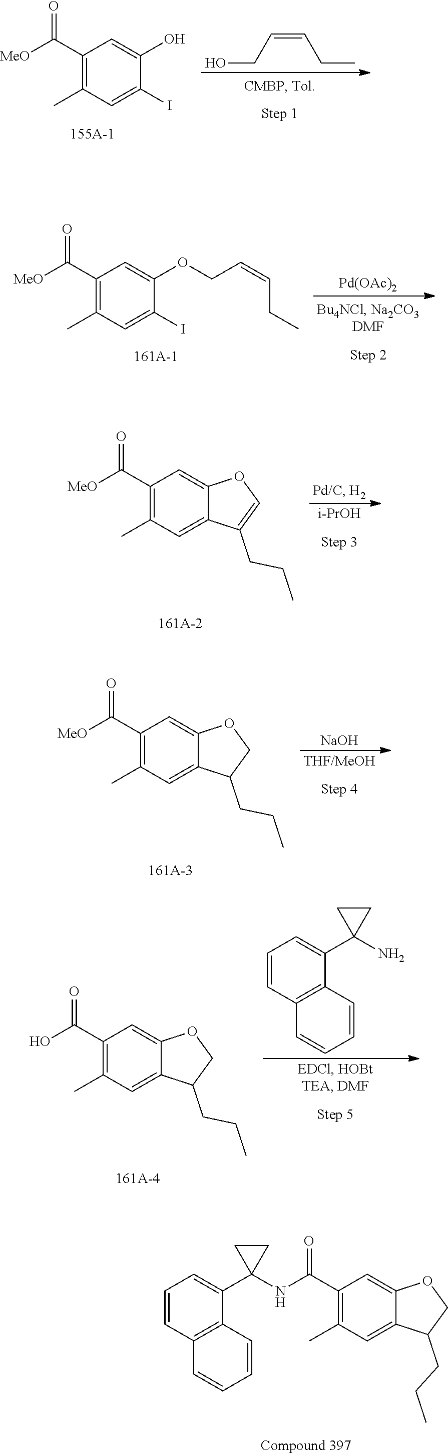

### Step 1: (Z)-Methyl 4-iodo-2-methyl-5-(pent-2-en-1-yloxy)benzoate (161A-1)

A mixture of methyl 5-hydroxy-4-iodo-2-methylbenzoate (500 mg, 1.71 mmol, 1.0 eq) and (Z)-pent-2-en-1-ol (221 mg, 2.57 mmol, 1.5 eq) in toluene (25 mL) was degassed and purged with N₂ three times. To the mixture was added CMBP (620 mg, 2.57 mmol, 1.5 eq) dropwise at 20° C. The mixture was stirred at 110° C. for 16 h. LCMS indicated that the starting material was completely consumed, and the desired mass was detected. The reaction mixture was allowed to cool to room temperature, poured into H₂O (30 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. (Z)-Methyl 4-iodo-2-methyl-5-(pent-2-en-1-yloxy)benzoate (400 mg, 1.11 mmol, 65% yield) was obtained as a colorless oil. M+H⁺=361.1 (LCMS); ¹H NMR (400 MHZ, DMSO-d₆) δ 7.77 (s, 1H), 7.35-7.27 (m, 1H), 5.70-5.50 (m, 2H), 4.73-4.58 (m, 2H), 3.84-3.78 (m, 3H), 2.44-2.35 (m, 3H), 2.17-2.08 (m, 2H), 1.00-0.94 (m, 3H).

### Step 2: Methyl 5-methyl-3-propylbenzofuran-6-carboxylate (161A-2)

A mixture of methyl(Z)-methyl 4-iodo-2-methyl-5-(pent-2-en-1-yloxy)benzoate (500 mg, 1.71 mmol, 1.0 eq) in DMF (25 mL) was degassed and purged with N₂ three times. To the mixture were added Pd(OAc) 2 (24.9 mg, 111 μmol, 0.1 eq), TBAC (340 mg, 1.22 mmol, 342 μL, 1.1 eq) and sodium carbonate (294 mg, 2.78 mmol, 2.5 eq). The mixture was stirred at 100° C. for 18 h. TLC indicated that the starting material was completely consumed. The reaction mixture was poured into H₂O (20 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. Methyl 5-methyl-3-propylbenzofuran-6-carboxylate (200 mg, 861 μmol, 78% yield) was obtained as a yellow oil. ¹H NMR (400 MHZ, DMSO-d₆) δ 7.97 (s, 1H), 7.91 (s, 1H), 7.54 (s, 1H), 3.87-3.81 (m, 3H), 2.64-2.57 (m, 5H), 1.74-1.59 (m, 2H), 0.96-0.91 (m, 3H).

### Step 3: Methyl 5-methyl-3-propyl-2,3-dihydrobenzofuran-6-carboxylate (161A-3)

To a solution of methyl 5-methyl-3-propylbenzofuran-6-carboxylate (160 mg, 689 μmol, 1.0 eq) in i-PrOH (10 mL) was added 10% palladium on carbon (100 mg). The mixture was degassed and purged with H₂ three times. The resulting mixture was stirred at 20° C. for 16 h under a H₂ (15 psi) atmosphere. LCMS indicated that the starting material was completely consumed, and the desired mass was detected. The combined organic layers were filtered through Celite pad and the filtrate was concentrated under vacuum to give the crude product methyl 5-methyl-3-propyl-2,3-dihydrobenzofuran-6-carboxylate (80.0 mg, 341 μmol, 50% yield) as a yellow oil. M+H⁺=235.1 (LCMS); ¹H NMR (400 MHz, CDCl₃) δ 7.31 (s, 1H), 7.03 (s, 1H), 4.65 (t, J=8.8 Hz, 1H), 4.22 (dd, J=6.6, 8.7 Hz, 1H), 3.87 (s, 3H), 3.52-3.35 (m, 1H), 2.53 (s, 3H), 1.82-1.71 (m, 1H), 1.61-1.32 (m, 3H), 0.97 (t, J=7.3 Hz, 3H).

### Step 4: 5-Methyl-3-propyl-2,3-dihydrobenzofuran-6-carboxylic acid (161A-4)

To a solution of methyl 5-methyl-3-propyl-2,3-dihydrobenzofuran-6-carboxylate (80.0 mg, 196 μmol, 1.0 eq) in a mixture of MeOH (1.6 mL) and THF (3.2 mL) was added NaOH (2 M aqueous, 3.6 mL, 21 eq). The mixture was stirred at 70° C. for 2 h. LCMS indicated that the starting material was completely consumed, and the desired mass was detected. The reaction mixture was allowed to cool to room temperature, poured into H₂O (10 mL) and washed with MTBE (3.0 mL×3). The aqueous layer was acidified to pH 5 with HCl (1 M aqueous). The product was extracted with DCM (5.0 mL×3). The combined organic layers were dried over Na₂SO₄, filtered, and concentrated under vacuum to give the crude product 5-methyl-3-propyl-2,3-dihydro benzofuran-6-carboxylic acid (80.0 mg) as a yellow oil, which was used in the next step without any further purification. ¹H NMR (400 MHz, DMSO-d₆) δ 12.57 (br s, 1H), 7.13 (d, J=3.4 Hz, 2H), 4.62 (t, J=8.9 Hz, 1H), 4.18 (dd, J=6.7, 8.8 Hz, 1H), 3.49-3.39 (m, 1H), 2.43 (s, 3H), 1.35 (s, 4H), 0.91 (t, J=7.2 Hz, 3H).

### Step 5: 5-Methyl-N-(1-(naphthalen-1-yl)cyclopropyl)-3-propyl-2,3-dihydrobenzofuran-6-carboxamide (Compound 397)

To a solution of 1-(naphthalen-1-yl)cyclopropanamine (58.2 mg, 318 μmol, 1.0 eq) and 5-methyl-3-propyl-2,3-dihydrobenzofuran-6-carboxylic acid (70.0 mg, 318 μmol, 1.0 eq) in DMF (8 mL) were added TEA (64.3 mg, 636 μmol, 88.5 μL, 2 eq), EDCI (73.1 mg, 381 μmol, 1.2 eq) and HOBt (51.5 mg, 381 μmol, 1.2 eq). The mixture was stirred at 20° C. for 16 h. LCMS indicated that the starting material was completely consumed, and the desired mass was detected. The reaction mixture was poured into H₂O (10 mL) and extracted with EtOAc (5.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 40%-70% B over 8 min; mobile phase A: 0.04% aqueous HCl, mobile phase B: acetonitrile). 5-Methyl-N-(1-(naphthalen-1-yl)cyclopropyl)-3-propyl-2,3-dihydrobenzofuran-6-carboxamide (45.5 mg, 117 μmol, 37% yield) was obtained as a white solid. M+H⁺=386.1 (LCMS); ¹H NMR (400 MHZ, DMSO-d₆) δ 9.01 (s, 1H), 8.64 (d, J=8.4 Hz, 1H), 7.92 (d, J=7.8 Hz, 1H), 7.81 (dd, J=7.7, 10.8 Hz, 2H), 7.60-7.42 (m, 3H), 6.96 (s, 1H), 6.41 (s, 1H), 4.54 (t, J=8.9 Hz, 1H), 4.11 (dd, J=6.4, 8.7 Hz, 1H), 3.39-3.33 (m, 1H), 1.95 (s, 3H), 1.67-1.56 (m, 1H), 1.46-1.24 (m, 5H), 1.16 (br d, J=3.5 Hz, 2H), 0.92-0.84 (m, 3H).

### Example 162: 6-Methyl-N-(1-(naphthalen-1-yl)cyclopropyl)-1H-indole-5-carboxamide (Compound 223)

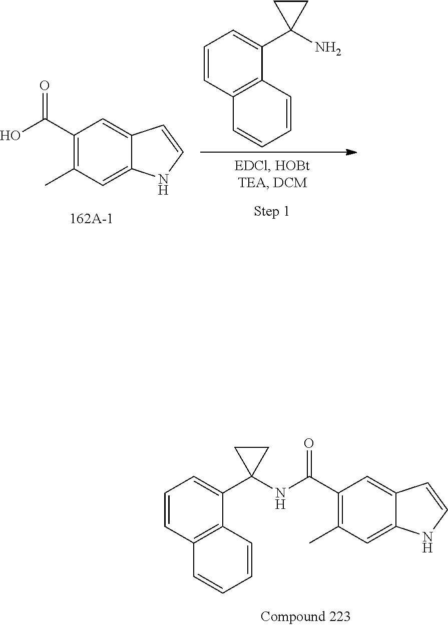 [SMILES: Cc1cc2[nH]ccc2cc1C(=O)O ; NC1(c2cccc3ccccc23)CC1 ; Cc1cc2[nH]ccc2cc1C(=O)NC1(c2cccc3ccccc23)CC1]

### Step 1: 6-Methyl-N-(1-(naphthalen-1-yl)cyclopropyl)-1H-indole-5-carboxamide (Compound 223)

To a mixture of 6-methyl-1H-indole-5-carboxylic acid (60.0 mg, 343 μmol, 1.0 eq) and 1-(naphthalen-1-yl)cyclopropanamine (62.8 mg, 343 μmol, 8.75 μL, 1.0 eq) in DCM (5.0 mL) was added TEA (104 mg, 1.03 mmol, 143 μL, 3.0 eq), followed by EDCI (98.5 mg, 514 μmol, 1.5 eq) and HOBt (69.4 mg, 514 μmol, 1.5 eq). The mixture was stirred at 20° C. for 16 h. LCMS indicated that the starting material was completely consumed, and the desired compound was detected. The reaction mixture was poured into H₂O (10 mL) and extracted with DCM (5.0 mL×2). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 45%-75% B over 8 min; mobile phase A: 0.2% aqueous FA, mobile phase B: acetonitrile). 6-Methyl-N-(1-(naphthalen-1-yl)cyclopropyl)-1H-indole-5-carboxamide (64.2 mg, 186 μmol, 54% yield) was obtained as a white solid. M+H⁺=341.1 (LCMS); ¹H NMR (400 MHZ, DMSO-d₆) δ 11.08-10.87 (m, 1H), 9.01-8.90 (m, 1H), 8.78-8.64 (m, 1H), 7.97-7.91 (m, 1H), 7.87-7.79 (m, 2H), 7.63-7.43 (m, 3H), 7.32-7.28 (m, 1H), 7.27-7.18 (m, 1H), 7.13-7.05 (m, 1H), 6.37-6.31 (m, 1H), 2.22-2.15 (m, 3H), 1.43-1.30 (m, 2H), 1.22-1.11 (m, 2H).

### Example 163: 2-(Aminomethyl)-5-methyl-N-(1-(naphthalen-1-yl)cyclopropyl)-1H-benzo[d]imidazole-6-carboxamide (Compound 280)

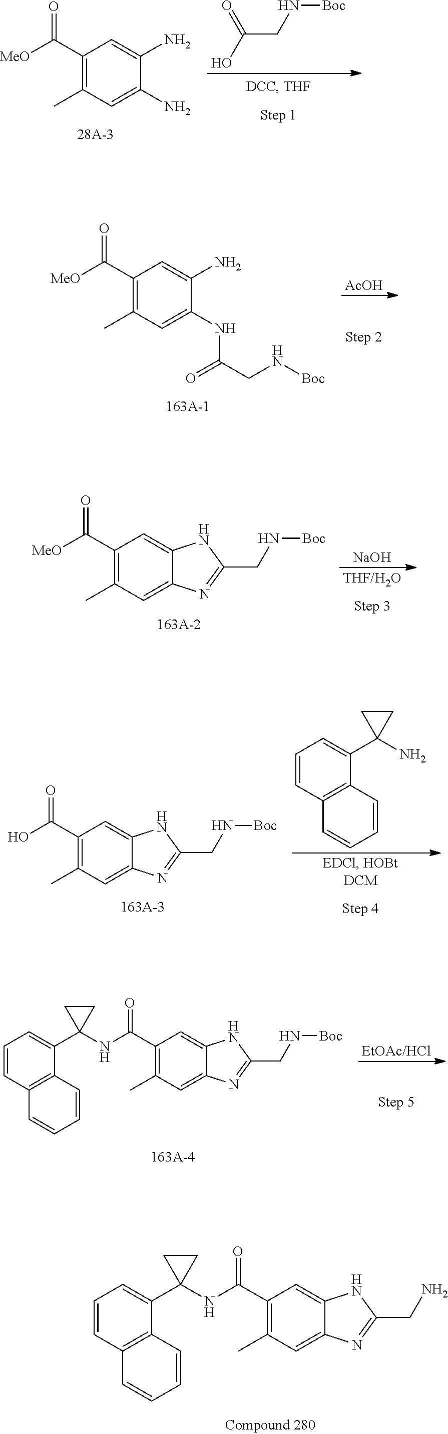

### Step 1: Methyl 5-amino-4-(2-((tert-butoxycarbonyl)amino) acetamido)-2-methylbenzoate (163A-1)

To a solution of methyl 4,5-diamino-2-methylbenzoate (300 mg, 1.66 mmol, 1.0 eq) and 2-((tert-butoxycarbonyl)amino) acetic acid (292 mg, 1.66 mmol, 1.0 eq) in THF (9.0 mL) was added N,N′-methanediylidenedicyclohexanamine (687 mg, 3.33 mmol, 674 μL, 2.0 eq). The mixture was stirred at 20° C. for 16 h. LCMS indicated that the starting material was completely consumed, and the desired mass was detected. The reaction mixture was poured into H₂O (10 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/4. Methyl 5-amino-4-(2-((tert-butoxycarbonyl)amino) acetamido)-2-methylbenzoate (200 mg, 593 mmol, 53% yield) was obtained as a white solid. M+H⁺=338.3 (LCMS); ¹H NMR (400 MHZ, CDCl₃) δ 7.95 (s, 1H), 7.80 (s, 1H), 6.56 (s, 1H), 5.40 (br t, J=5.7 Hz, 1H), 3.94 (d, J=5.7 Hz, 2H), 3.81 (s, 3H), 2.51 (s, 3H), 1.48 (s, 9H).

### Step 2: Methyl 2-(((tert-butoxycarbonyl)amino)methyl)-5-methyl-1H-benzo[d]imidazole-6-carboxylate (163A-2)

A solution of methyl 5-amino-4-(2-((tert-butoxycarbonyl)amino) acetamido)-2-methylbenzoate (200 mg, 593 μmol, 1.0 eq) in acetic acid (7.0 mL) was stirred at 80° C. for 4 h. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give the crude product methyl 2-(((tert-butoxycarbonyl)amino)methyl)-5-methyl-1H-benzo[d]imidazole-6-carboxylate (200 mg) was obtained as a white solid. M+H⁺=320.3 (LCMS).

### Step 3: 2-(((tert-Butoxycarbonyl)amino)methyl)-5-methyl-1H-benzo[d]imidazole-6-carboxylic acid (163A-3)

To a solution of methyl 2-(((tert-butoxycarbonyl)amino)methyl)-5-methyl-1H-benzo[d]imidazole-6-carboxylate (180 mg, 564 μmol, 1.0 eq) in a mixture of H₂O (3.0 mL) and THF (9.0 mL) was added NaOH (2 M aqueous, 845 μL, 3.0 eq). The mixture was stirred at 80° C. for 16 h. LCMS indicated that the starting material was completely consumed, and the desired mass was detected. The reaction mixture was allowed to cool to room temperature, poured into H₂O (15 mL) and extracted with MTBE (15 mL×2). The aqueous layer was acidified to pH 5 using HCl (1 M aqueous). The product was extracted with EtOAc (5.0 mL×5). The combined organic layers were dried over Na₂SO₄, filtered, and concentrated under vacuum to give the crude product 2-(((tert-butoxycarbonyl)amino)methyl)-5-methyl-1H-benzo[d]imidazole-6-carboxylic acid (150 mg), which was used in the next step without any further purification. M+H⁺=306.3 (LCMS).

### Step 4: tert-Butyl((5-methyl-6-((1-(naphthalen-1-yl)cyclopropyl)carbamoyl)-1H-benzo[d]imidazol-2-yl)methyl)carbamate (163A-4)

To a solution of 1-(naphthalen-1-yl)cyclopropanamine (32.7 mg, 179 μmol, 0.8 eq) and 2-(((tert-butoxycarbonyl)amino)methyl)-5-methyl-1H-benzo[d]imidazole-6-carboxylic acid (100 mg, 328 μmol, 1.0 eq) in DCM (10 mL) were added TEA (45.2 mg, 446 μmol, 62.1 μL, 2.0 eq), EDCI (51.3 mg, 268 μmol, 1.2 eq) and HOBt (36.2 mg, 268 μmol, 1.2 eq). The mixture was stirred at 20° C. for 18 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by preparative TLC (DCM/MeOH=10/1, R_{f}=0.7). tert-Butyl((5-methyl-6-((1-(naphthalen-1-yl)cyclopropyl)carbamoyl)-1H-benzo[d]imidazol-2-yl)methyl)carbamate (100 mg, crude) was obtained as a colorless oil. M+H⁺=471.3 (LCMS).

### Step 5: 2-(Aminomethyl)-5-methyl-N-(1-(naphthalen-1-yl)cyclopropyl)-1H-benzo[d]imidazole-6-carboxamide (Compound 280)

To a stirred solution of tert-butyl((5-methyl-6-((1-(naphthalen-1-yl)cyclopropyl)carbamoyl)-1H-benzo[d]imidazol-2-yl)methyl)carbamate (30.0 mg, 63.8 μmol, 1.0 eq) in EtOAc (1.0 mL) was added HCl/EtOAc (4 M, 3.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Gemini C18 column (80×30 mm, 3 μm); flow rate: 25 mL/min; gradient: 1%-50% B over 8 min; mobile phase A: 0.04% aqueous HCl, mobile phase B: acetonitrile). 2-(Aminomethyl)-5-methyl-N-(1-(naphthalen-1-yl)cyclopropyl)-1H-benzo[d]imidazole-6-carboxamide (11.7 mg, 31.6 μmol, 50% yield, HCl salt) was obtained as a white solid. M+H⁺=371.1 (LCMS); ¹H NMR (400 MHZ, DMSO-d₆) δ 9.15 (s, 1H), 8.73-8.54 (m, 4H), 7.95 (d, J=7.8 Hz, 1H), 7.83 (dd, J=2.7, 7.7 Hz, 2H), 7.62-7.51 (m, 2H), 7.47 (t, J=7.6 Hz, 1H), 7.36 (s, 1H), 7.31 (s, 1H), 4.30 (br s, 2H), 2.19 (s, 3H), 1.39 (s, 2H), 1.23-1.14 (m, 2H).

### Example 164: 2-(Aminomethyl)-6-methyl-N-(1-(naphthalen-1-yl)cyclopropyl)benzo[d]thiazole-5-carboxamide (Compound 245)

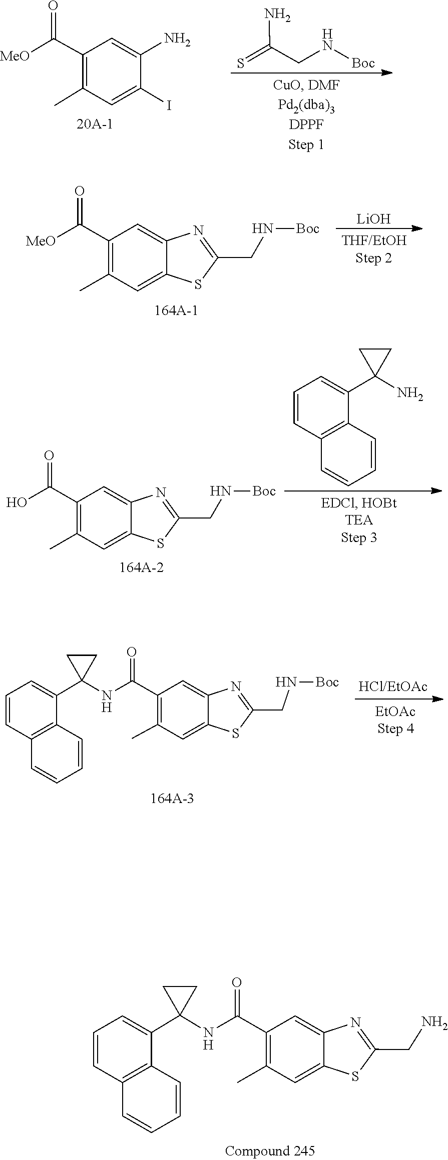

### Step 1: Methyl 2-(((tert-butoxycarbonyl)amino)methyl)-6-methylbenzo[d]thiazole-5-carboxylate (164A-1)

A mixture of methyl 5-amino-4-iodo-2-methylbenzoate (300 mg, 1.03 mmol, 1.0 eq), tert-butyl(2-amino-2-thioxoethyl)carbamate (196 mg, 1.03 mmol, 1.0 eq) and CuO (82.0 mg, 1.03 mmol, 1.0 eq) in DMF (6.0 mL) was degassed and purged with N₂ three times. Pd₂ (dba) 3 (18.9 mg, 21.6 μmol, 1.1 eq) and DPPF (28.6 mg, 51.5 μmol, 0.05 eq) were added and the mixture was stirred at 60° C. for 16 h under a N₂ atmosphere. LCMS indicated that the starting material was completely consumed, and the desired mass was detected. The reaction mixture was allowed to cool to room temperature, poured into H₂O (30 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/100 to 1/3. Methyl 2-(((tert-butoxycarbonyl)amino)methyl)-6-methylbenzo[d]thiazole-5-carboxylate (340 mg, 1.11 mmol, 99% yield) was obtained as a white solid. M+H⁺=337.2 (LCMS); ¹H NMR (400 MHZ, DMSO-d₆) δ=8.32 (s, 1H), 8.04 (s, 1H), 7.90 (s, 1H), 4.51 (br d, J=6.1 Hz, 2H), 3.87 (s, 3H), 2.61 (s, 3H), 1.42 (s, 9H).

### Step 2: 2-(((tert-Butoxycarbonyl)amino)methyl)-6-methylbenzo[d]thiazole-5-carboxylic acid (164A-2)

To a solution of methyl 2-(((tert-butoxycarbonyl)amino)methyl)-6-methylbenzo[d]thiazole-5-carboxylate (340 mg, 1.11 mmol, 1.0 eq) in a mixture of THF (10 mL) and H₂O (5.0 mL) was added LiOH·H₂O (102 mg, 2.71 mmol, 2.5 eq). The mixture was stirred at 20° C. for 16 h. LCMS indicated that the starting material was completely consumed, and the desired mass was detected. The mixture was poured into H₂O (10 mL) and washed with MTBE (15 mL×2). The aqueous layer was acidified to pH 5 with HCl (1 M aqueous). The product was extracted with EtOAc (8.0 mL×3). The combined organic layers were dried over Na₂SO₄, filtered, and concentrated under vacuum to give the crude product 2-(((tert-butoxycarbonyl)amino)methyl)-6-methylbenzo[d]thiazole-5-carboxylic acid (340 mg), which was used in the next step without any further purification. M+H⁺=323.0 (LCMS).

### Step 3: tert-Butyl((6-methyl-5-((1-(naphthalen-1-yl)cyclopropyl)carbamoyl)benzo[d]thiazol-2-yl)methyl)carbamate (164A-3)

To a solution of 1-(naphthalen-1-yl)cyclopropanamine (56.8 mg, 310 μmol, 1.0 eq) and 2-(((tert-butoxycarbonyl)amino)methyl)-6-methylbenzo[d]thiazole-5-carboxylic acid (100 mg, 310 μmol, 1.0 eq) in DCM (6.0 mL) were added TEA (94.2 mg, 931 μmol, 130 μL, 3.0 eq), EDCI (119 mg, 620 μmol, 2.0 eq) and HOBt (83.8 mg, 620 μmol, 2.0 eq). The mixture was stirred at 20° C. for 15 h. LCMS indicated that the starting material was completely consumed, and the desired mass was detected. The reaction mixture was poured into H₂O (10 mL) and extracted with DCM (10 mL×2). The combined organic layers were dried over Na₂SO₄, filtered, and concentrated under vacuum to give the crude product tert-butyl((6-methyl-5-((1-(naphthalen-1-yl)cyclopropyl)carbamoyl)benzo[d]thiazol-2-yl)methyl)carbamate (110 mg), which was used in the next step without any further purification. M+H⁺=488.3 (LCMS).

### Step 4: 2-(Aminomethyl)-6-methyl-N-(1-(naphthalen-1-yl)cyclopropyl)benzo[d]thiazole-5-carboxamide (Compound 245)

To a stirred solution of tert-butyl((6-methyl-5-((1-(naphthalen-1-yl)cyclopropyl)carbamoyl)benzo[d]thiazol-2-yl)methyl)carbamate (100 mg, 205 μmol, 1.0 eq) in EtOAc (3.0 mL) was added HCl/EtOAc (4 M, 3.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Gemini C18 column (80×40 mm, 3 μm); flow rate: 40 mL/min; gradient: 5%-35% B over 7 min; mobile phase A: 0.04% aqueous HCl, mobile phase B: acetonitrile). 2-(Aminomethyl)-6-methyl-N-(1-(naphthalen-1-yl)cyclopropyl)benzo[d]thiazole-5-carboxamide (74.7 mg, 173 μmol, 84% yield, HCl salt) was obtained as a white solid. M+H⁺=388.0 (LCMS); ¹H NMR (400 MHZ, DMSO-d₆) δ=9.32 (s, 1H), 8.78-8.53 (m, 4H), 7.99-7.93 (m, 2H), 7.85 (t, J=7.9 Hz, 2H), 7.65-7.45 (m, 4H), 4.58 (br s, 2H), 2.21 (s, 3H), 1.46-1.20 (m, 4H).

### Example 165: 6-Methyl-N-(1-(naphthalen-1-yl)cyclopropyl)-2-oxo-2,3-dihydro-1H-benzo[d]imidazole-5-carboxamide (Compound 238)

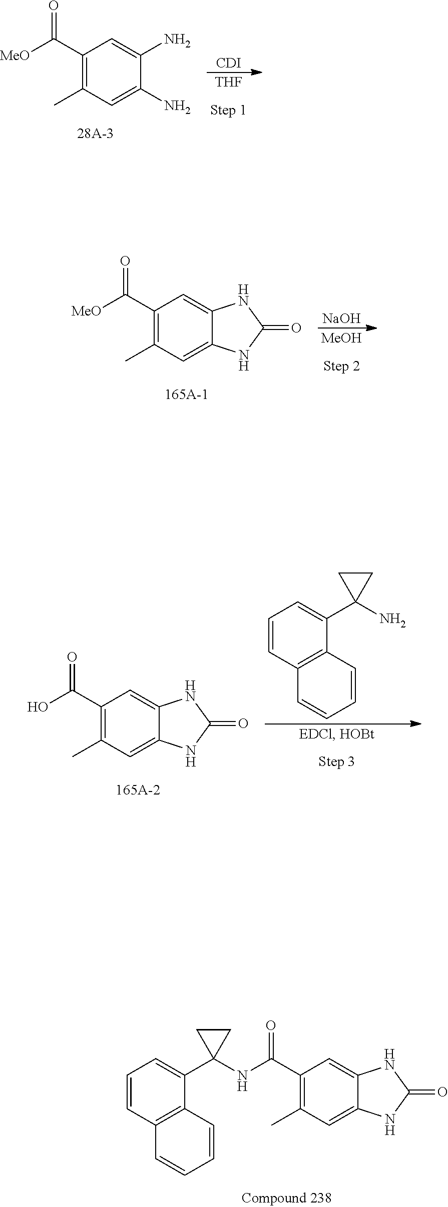

### Step 1: Methyl 6-methyl-2-oxo-2,3-dihydro-1H-benzo[d]imidazole-5-carboxylate (165A-1)

To a solution of methyl 4,5-diamino-2-methylbenzoate (400 mg, 2.22 mmol, 1.0 eq) in MeCN (40 mL) was added CDI (396 mg, 2.44 mmol, 1.1 eq). The resulting mixture was stirred at 70° C. for 2 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. Methyl 6-methyl-2-oxo-2,3-dihydro-1H-benzo[d]imidazole-5-carboxylate (340 mg, 1.65 mmol, 74% yield) was obtained as a white solid. M+H⁺=207.2 (LCMS); ¹H NMR (400 MHZ, DMSO-d₆) δ 10.89 (br s, 1H), 10.71 (s, 1H), 7.41 (s, 1H), 6.84 (s, 1H), 3.78 (s, 3H), 2.55-2.52 (m, 3H).

### Step 2: 6-Methyl-2-oxo-2,3-dihydro-1H-benzo[d]imidazole-5-carboxylic acid (165A-2)

To a solution of methyl 6-methyl-2-oxo-2,3-dihydro-1H-benzo[d]imidazole-5-carboxylate (120 mg, 582 μmol, 1.0 eq) in MeOH (5.0 mL) was added NaOH (2 M aqueous, 1.08 mL, 3.5 eq). The mixture was stirred at 50° C. for 4 h. LCMS indicated that the starting material was completely consumed, and the desired mass was detected. The mixture was poured into H₂O (3.0 mL) and extracted with MTBE (5.0 mL×2). The aqueous was acidified to pH 5 using HCl (1 M aqueous). The product was extracted with EtOAc (5.0 mL×5) and the combined organic layers were dried over Na₂SO₄, filtered, and concentrated under vacuum to give the crude product 6-methyl-2-oxo-2,3-dihydro-1H-benzo[d]imidazole-5-carboxylic acid (110 mg) as a white solid. M+H⁺=193.2 (LCMS).

### Step 3: 6-Methyl-N-(1-(naphthalen-1-yl)cyclopropyl)-2-oxo-2,3-dihydro-1H-benzo[d]imidazole-5-carboxamide (Compound 238)

To a solution of 1-(naphthalen-1-yl)cyclopropanamine (105 mg, 572 μmol, 1.0 eq) and 6-methyl-2-oxo-2,3-dihydro-1H-benzo[d]imidazole-5-carboxylic acid (110 mg, 572 μmol, 1.0 eq) in DCM (18 mL) were added TEA (174 mg, 1.72 mmol, 239 μL, 3.0 eq), EDCI (219 mg, 1.14 mmol, 2.0 eq) and HOBt (155 mg, 1.14 mmol, 2.0 eq). The mixture was stirred at 25° C. for 16 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 25%-65% B over 8 min; mobile phase A: 0.2% aqueous FA, mobile phase B: acetonitrile). 6-Methyl-N-(1-(naphthalen-1-yl)cyclopropyl)-2-oxo-2,3-dihydro-1H-benzo[d]imidazole-5-carboxamide (20.0 mg, 56.0 μmol, 10% yield) was obtained as a white solid. M+H⁺=358.1 (LCMS); ¹H NMR (400 MHZ, DMSO-d₆) δ 10.59 (s, 1H), 10.49 (s, 1H), 8.99 (s, 1H), 8.67 (d, J=8.4 Hz, 1H), 7.93 (d, J=7.4 Hz, 1H), 7.85-7.78 (m, 2H), 7.62-7.42 (m, 3H), 6.64 (d, J=13.0 Hz, 2H), 2.07 (s, 3H), 1.40-1.29 (m, 2H), 1.20-1.08 (m, 2H).

### Example 166: 1,3,6-Trimethyl-N-(1-(naphthalen-1-yl)cyclopropyl)-2-oxo-2,3-dihydro-1H-benzo[d]imidazole-5-carboxamide (Compound 237)

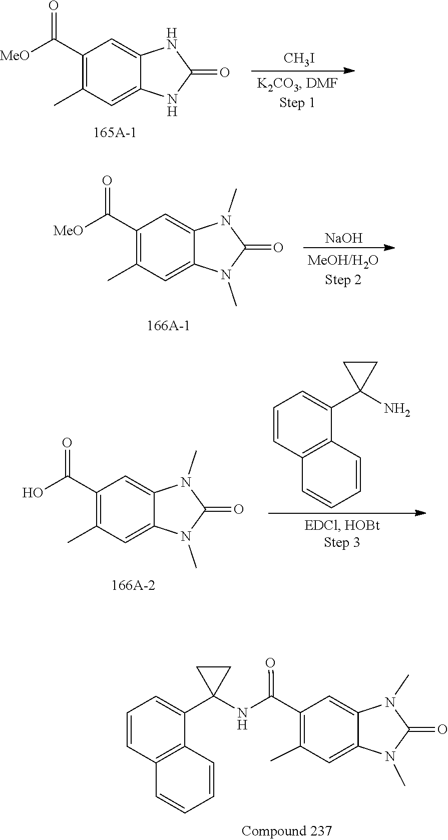

### Step 1: Methyl 1,3,6-trimethyl-2-oxo-2,3-dihydro-1H-benzo[d]imidazole-5-carboxylate (166A-1)

To a solution of methyl 6-methyl-2-oxo-2,3-dihydro-1H-benzo[d]imidazole-5-carboxylate (100 mg, 485 μmol, 1.0 eq) in DMF (5.0 mL) were added CH₃I (413 mg, 2.91 mmol, 181 μL, 6.0 eq) and K₂CO₃ (402 mg, 2.91 mmol, 6.0 eq). The mixture was stirred at 25° C. for 16 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by preparative TLC (EtOAc/petroleum ether=1/1, R_{f}=0.2). Methyl 1,3,6-trimethyl-2-oxo-2,3-dihydro-1H-benzo[d]imidazole-5-carboxylate (88.0 mg, 376 μmol, 77% yield) was obtained as a white solid. M+H⁺=235.2 (LCMS); 1H NMR (400 MHZ, DMSO-d₆) δ 7.59 (s, 1H), 7.11 (s, 1H), 3.82 (s, 3H), 3.34 (s, 6H), 2.58 (s, 3H).

### Step 2: 1,3,6-Trimethyl-2-oxo-2,3-dihydro-1H-benzo[d]imidazole-5-carboxylic acid (166A-2)

To a solution of methyl 1,3,6-trimethyl-2-oxo-2,3-dihydro-1H-benzo[d]imidazole-5-carboxylate (70.0 mg, 299 μmol, 1.0 eq) in MeOH (3.5 mL) was added NaOH (2 M aqueous, 523 μL, 3.5 eq). The mixture was stirred at 50° C. for 3 h. LCMS indicated that the starting material was completely consumed, and the desired mass was detected. The mixture was poured into H₂O (3.0 mL) and extracted with MTBE (5.0 mL×2). The aqueous was acidified to pH 5 using HCl (1 M aqueous). The product was extracted with EtOAc (5.0 mL×5), the combined organic layers were dried over Na₂SO₄, filtered, and concentrated under vacuum to give the crude product 1,3,6-trimethyl-2-oxo-2,3-dihydro-1H-benzo[d]imidazole-5-carboxylic acid (60.0 mg) as a white solid. M+H⁺=221.0 (LCMS).

### Step 3: 1,3,6-Trimethyl-N-(1-(naphthalen-1-yl)cyclopropyl)-2-oxo-2,3-dihydro-1H-benzo[d]imidazole-5-carboxamide (Compound 237)

To a solution of 1-(naphthalen-1-yl)cyclopropanamine (83.2 mg, 454 μmol, 1.0 eq) and 1,3,6-trimethyl-2-oxo-2,3-dihydro-1H-benzo[d]imidazole-5-carboxylic acid (100 mg, 454 μmol, 1.0 eq) in DCM (15 mL) were added TEA (138 mg, 1.36 mmol, 190 μL, 3.0 eq), EDCI (174 mg, 908 μmol, 2.0 eq) and HOBt (123 mg, 908 μmol, 2.0 eq). The mixture was stirred at 25° C. for 16 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 30%-65% B over 8 min; mobile phase A: 0.2% aqueous FA, mobile phase B: acetonitrile). 1,3,6-Trimethyl-N-(1-(naphthalen-1-yl)cyclopropyl)-2-oxo-2,3-dihydro-1H-benzo[d]imidazole-5-carboxamide (36.8 mg, 95.5 μmol, 21% yield) was obtained as a white solid. M+H⁺=386.1 (LCMS); ¹H NMR (400 MHZ, DMSO-d₆) δ 9.00 (s, 1H), 8.69 (d, J=8.3 Hz, 1H), 7.93 (d, J=7.6 Hz, 1H), 7.84 (dd, J=3.7, 7.6 Hz, 2H), 7.61-7.44 (m, 3H), 6.91 (d, J=5.0 Hz, 2H), 3.26 (d, J=8.4 Hz, 6H), 2.10 (s, 3H), 1.42-1.34 (m, 2H), 1.23-1.15 (m, 2H).

### Example 167: 7-Methyl-N-(1-(naphthalen-1-yl)cyclopropyl)-1,2,3,4-tetrahydroquinoxaline-6-carboxamide (Compound 297)

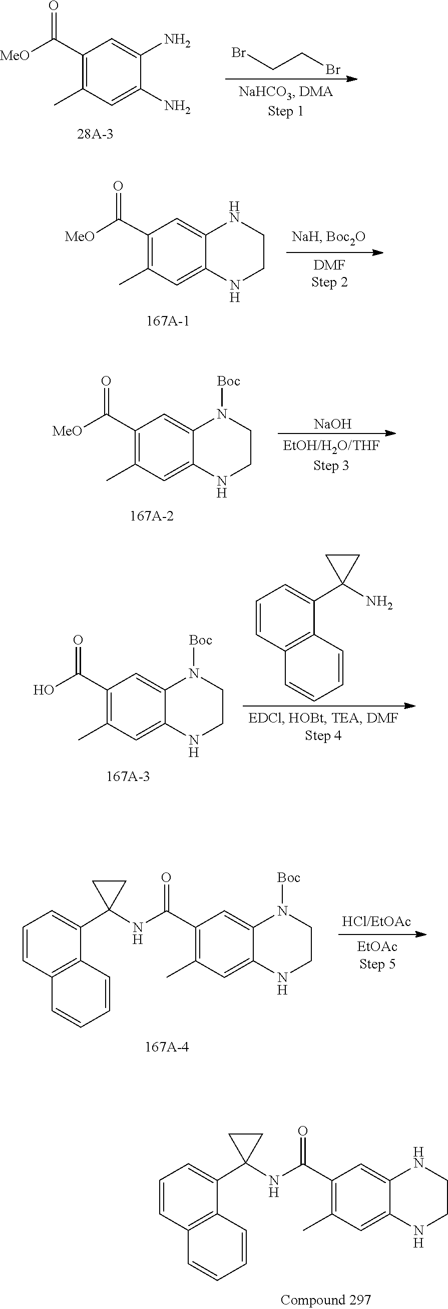

### Step 1: Methyl 7-methyl-1,2,3,4-tetrahydroquinoxaline-6-carboxylate (167A-1)

To a solution of methyl 4,5-diamino-2-methylbenzoate (400 mg, 2.22 mmol, 1.0 eq) in DMA (8.0 mL) were added NaHCO₃ (1.86 g, 22.2 mmol, 10 eq) and 1,2-dibromoethane (500 mg, 2.66 mmol, 200 μmL, 1.2 eq). The mixture was stirred at 80° C. for 16 h. LCMS indicated that the starting material was completely consumed, and the desired mass was detected. The reaction mixture was allowed to cool to room temperature, poured into H₂O (15 mL) and extracted with EtOAc (10 mL×5). The combined organic layers were washed with brine (20 mL×2), dried over Na₂SO₄, filtered, and concentrated under vacuum to give a residue which was purified by flash silica gel chromatography using a gradient of EtOAc/petroleum ether from 0/1 to 1/0. Methyl 7-methyl-1,2,3,4-tetrahydro quinoxaline-6-carboxylate (200 mg, 969 μmol, 44% yield) was obtained as a yellow solid. M+H⁺=207.1 (LCMS); ¹H NMR (400 MHz, DMSO-d₆) δ 7.01 (s, 1H), 6.18 (s, 1H), 6.12 (br s, 1H), 5.36 (br s, 1H), 3.67 (s, 3H), 3.26 (br d, J=2.8 Hz, 2H), 3.16-3.10 (m, 2H), 1.96 (s, 3H).

### Step 2: 1-tert-Butyl 7-methyl 6-methyl-3,4-dihydroquinoxaline-1,7(2H)-dicarboxylate (167A-2)

To a solution of methyl 7-methyl-1,2,3,4-tetrahydroquinoxaline-6-carboxylate (200 mg, 969 μmol, 1.0 eq) in DMF (20 mL) was added sodium hydride (96.9 mg, 2.42 mmol, 60% purity, 2.5 eq) at 0° C., followed by di-tert-butyl dicarbonate (529 mg, 2.42 mmol, 557 μL, 2.5 eq). The resulting mixture was stirred at 25° C. for 16 h. TLC indicated that the starting material was completely consumed. The reaction mixture was poured into H₂O (10 mL) and extracted with EtOAc (10 mL×3). The combined organic layers were washed with brine (10 mL×2), dried over Na₂SO₄, filtered, and concentrated under vacuum to give a residue which was purified by flash silica gel chromatography using a gradient of EtOAc/petroleum ether from 0/1 to 1/1. 1-tert-Butyl 7-methyl 6-methyl-3,4-dihydroquinoxaline-1,7(2H)-dicarboxylate (150 mg, 489 μmol, 50% yield) was obtained as a yellow gum. ¹H NMR (400 MHZ, DMSO-d₆) δ 7.99 (s, 1H), 6.82 (br s, 1H), 6.39 (s, 1H), 3.71 (s, 3H), 3.58 (br t, J=4.9 Hz, 2H), 3.28 (br d, J=3.0 Hz, 2H), 2.38 (s, 3H), 1.46 (s, 9H).

### Step 3: 4-(tert-Butoxycarbonyl)-7-methyl-1,2,3,4-tetrahydroquinoxaline-6-carboxylic acid (167A-3)

To a solution of 1-tert-butyl 7-methyl 6-methyl-3,4-dihydroquinoxaline-1,7(2H)-dicarboxylate (150 mg, 489 μmol, 1.0 eq) in a mixture of THF (7.5 mL) and MeOH (7.5 mL) was added NaOH (2 M aqueous, 1.22 mL, 5.0 eq). The resulting mixture was stirred at 70° C. for 16 h. LCMS indicated that the starting material was completely consumed, and the desired mass was detected. The reaction mixture was poured into H₂O (10 mL) and adjust the pH to 6 using HCl (1 M aqueous), and a precipitate was formed. The mixture was filtered, and the filter cake was washed with H₂O (5.0 mL) and dried under vacuum to give 4-(tert-butoxycarbonyl)-7-methyl-1,2,3,4-tetrahydroquinoxaline-6-carboxylic acid (100 mg, 342 μmol, 70% yield) as an orange solid, which was used in the next step without any further purification. M−H⁻=291.0 (LCMS).

### Step 4: tert-Butyl 6-methyl-7-((1-(naphthalen-1-yl)cyclopropyl)carbamoyl)-3,4-dihydro quinoxaline-1 (2H)-carboxylate (167A-4)

To a solution of 4-(tert-butoxycarbonyl)-7-methyl-1,2,3,4-tetrahydroquinoxaline-6-carboxylic acid (100 mg, 342 μmol, 1.0 eq) and 1-(naphthalen-1-yl)cyclopropanamine (62.7 mg, 342 μmol, 1.0 eq) in DMF (10 mL) were added TEA (104 mg, 1.03 mmol, 143 μL, 3.0 eq), EDCI (78.7 mg, 411 μmol, 1.2 eq) and HOBt (55.5 mg, 411 μmol, 1.2 eq). The mixture was stirred at 25° C. for 16 h. LCMS indicated that the starting material was completely consumed, and the desired mass was detected. The reaction mixture was poured into H₂O (10 mL), and a precipitate was formed. The mixture was filtered and the filter cake was washed with H₂O (5.0 mL) and dried under vacuum to give tert-butyl 6-methyl-7-((1-(naphthalen-1-yl)cyclopropyl)carbamoyl)-3,4-dihydroquinoxaline-1 (2H)-carboxylate (130 mg, 284 μmol, 83% yield) as an orange solid. M+H⁺=458.2 (LCMS).

### Step 5: 7-Methyl-N-(1-(naphthalen-1-yl)cyclopropyl)-1,2,3,4-tetrahydroquinoxaline-6-carboxamide (Compound 297)

To a solution of tert-butyl 6-methyl-7-((1-(naphthalen-1-yl)cyclopropyl)carbamoyl)-3,4-dihydroquinoxaline-1 (2H)-carboxylate (130 mg, 284 μmol, 1.0 eq) in EtOAc (3.0 mL) was added HCl/EtOAc (4 M, 10 mL). The resulting mixture was stirred at 25° C. for 1 h. LCMS indicated that the starting material was completely consumed, and the desired mass was detected. The reaction mixture was concentrated at 30° C. under vacuum to give a residue which was purified by preparative HPLC (Phenomenex luna C18 column (80×40 mm, 3 μm); flow rate: 25 mL/min; gradient: 5%-40% B over 7 min; mobile phase A: 0.04% aqueous HCl, mobile phase B: acetonitrile). 7-Methyl-N-(1-(naphthalen-1-yl)cyclopropyl)-1,2,3,4-tetrahydroquinoxaline-6-carboxamide (35.9 mg, 90.5 μmol, 32% yield, HCl salt) was obtained as a yellow solid. M+H⁺=358.1 (LCMS); ¹H NMR (400 MHZ, DMSO-d₆) δ 8.93 (br s, 1H), 8.65 (d, J=8.4 Hz, 1H), 7.93 (d, J=7.9 Hz, 1H), 7.87-7.74 (m, 2H), 7.63-7.37 (m, 3H), 6.83-6.66 (m, 1H), 6.46 (br s, 1H), 3.40-3.25 (m, 4H), 1.99 (s, 3H), 1.32 (br s, 2H), 1.15 (br s, 2H).

### Example 168: 6-Methyl-N-(1-(naphthalen-1-yl)cyclopropyl)-1,2,3,4-tetrahydroquinoline-7-carboxamide (Compound 227)

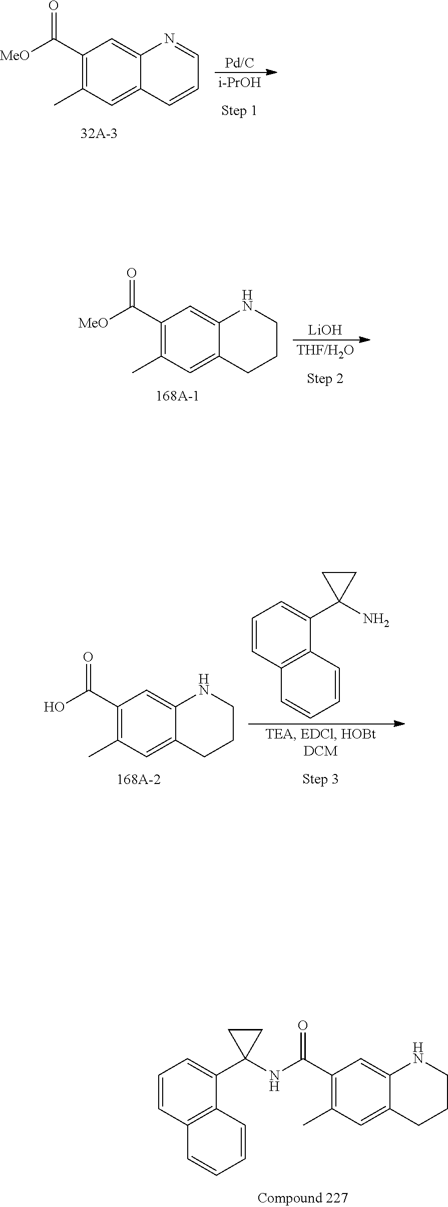

### Step 1: Methyl 6-methyl-1,2,3,4-tetrahydroquinoline-7-carboxylate (168A-1)

To a solution of methyl 6-methylquinoline-7-carboxylate (51.0 mg, 254 μmol, 1.0 eq) in i-PrOH (12 mL) was added 10% palladium on carbon (10 mg, 254 μmol, 1.0 eq) at 20° C. under a N₂ atmosphere. The mixture was degassed and purged with H₂ three times, then stirred at 50° C. for 4 h under a H₂ (50 psi) atmosphere. LCMS indicated that the starting material was completely consumed, and the desired mass was detected. The suspension was allowed to cool to room temperature, filtered through a pad of Celite and the filter cake was washed with i-PrOH (4.0 mL×3). The combined filtrates were concentrated to give the crude product methyl 6-methyl-1,2,3,4-tetrahydroquinoline-7-carboxylate (70.0 mg), which was used in the next step without any further purification. M+H⁺=206.2 (LCMS).

### Step 2: 6-Methyl-1,2,3,4-tetrahydroquinoline-7-carboxylic acid (168A-2)

To a solution of methyl 6-methyl-1,2,3,4-tetrahydroquinoline-7-carboxylate (70.0 mg, 341 μmol, 1.0 eq) in THF (2.0 mL) was added a mixture of LiOH (24.5 mg, 1.02 mmol, 3.0 eq) in H₂O (1.0 mL). The mixture was stirred at 20° C. for 16 h. LCMS indicated that the starting material was completely consumed, and the desired mass was detected. The reaction mixture was poured into H₂O (3.0 mL) and extracted with MTBE (1.0 mL×3). The aqueous layer was basified to pH 5 using HCl (1 M aqueous) and extracted with EtOAc (1.0 mL×3). The combined organic layers were dried over Na₂SO₄, filtered, and concentrated under vacuum to give the crude product 6-methyl-1,2,3,4-tetrahydroquinoline-7-carboxylic acid (50.0 mg), which was used in the next step without any further purification. M+H⁺=192.1 (LCMS).

### Step 3: 6-Methyl-N-(1-(naphthalen-1-yl)cyclopropyl)-1,2,3,4-tetrahydroquinoline-7-carb oxamide (Compound 227)

To a solution of 6-methyl-1,2,3,4-tetrahydroquinoline-7-carboxylic acid (26.1 mg, 136 μmol, 1.0 eq) and 1-(naphthalen-1-yl)cyclopropanamine (25.0 mg, 136 μmol, 1.0 eq) in DCM (5.0 mL) were added TEA (41.1 mg, 409 μmol, 57.0 μL, 3.0 eq), EDCI (65.4 mg, 341 μmol, 1.5 eq) and HOBt (46.1 mg, 341 μmol, 1.5 eq). The mixture was stirred at 20° C. for 2 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 15%-45% B over 8 min; mobile phase A: 0.04% aqueous HCl, mobile phase B: acetonitrile). 6-Methyl-N-(1-(naphthalen-1-yl)cyclopropyl)-1,2,3,4-tetrahydro quinoline-7-carboxamide (11.5 mg, 28.2 μmol, 10% yield, HCl salt) was obtained as a white solid. M+H⁺=357.2 (LCMS); ¹H NMR (400 MHZ, DMSO-d₆) δ 9.04 (br s, 1H), 8.63 (d, J=8.3 Hz, 1H), 7.96-7.90 (m, 1H), 7.86-7.77 (m, 2H), 7.61-7.42 (m, 3H), 6.88-6.78 (m, 1H), 6.61-6.49 (m, 1H), 3.25-3.12 (m, 3H), 1.93-1.71 (s, 6H), 1.33 (br s, 2H), 1.22-1.11 (m, 2H).

### Example 169: 7-Methyl-N-(1-(naphthalen-1-yl)cyclopropyl)-3-oxo-1,2,3,4-tetrahydrocyclopenta[b]indole-6-carboxamide (Compound 334)

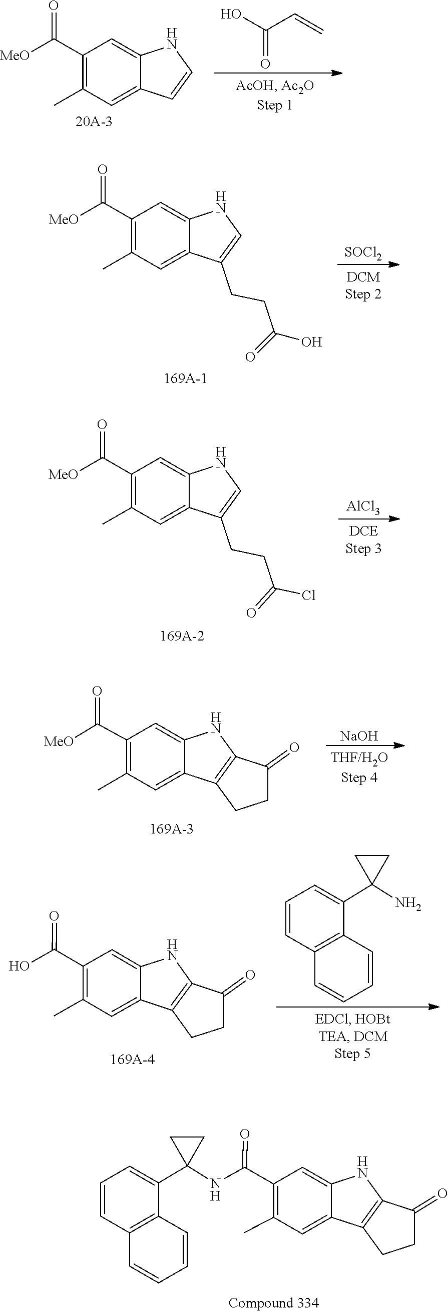

### Step 1: 3-(6-(Methoxycarbonyl)-5-methyl-1H-indol-3-yl)propanoic acid (169A-1)

To a solution of methyl 5-methyl-1H-indole-6-carboxylate (400 mg, 2.11 mmol, 1.0 eq) in AcOH (2 mL) were added acetyl acetate (432 mg, 4.23 mmol, 396 μL, 2.0 eq) and acrylic acid (381 mg, 5.29 mmol, 2.5 eq). The mixture was stirred at 50° C. for 72 h. LCMS indicated that 40% of the starting material remained and 50% of the desired mass was detected. The reaction mixture was concentrated under vacuum to give a residue which was purified by flash silica gel chromatography using a gradient of EtOAc/petroleum ether from 0/1 to 1/1. 3-(6-(Methoxycarbonyl)-5-methyl-1H-indol-3-yl)propanoic acid (200 mg, 765 μmol, 36% yield) was obtained as a pale yellow solid. M+H⁺=262.2 (LCMS).

### Step 2: Methyl 3-(3-chloro-3-oxopropyl)-5-methyl-1H-indole-6-carboxylate (169A-2)

To a solution of 3-(6-methoxycarbonyl-5-methyl-1H-indol-3-yl)propanoic acid (170 mg, 651 μmol, 1.0 eq) in DCM (4.0 mL) was added SOCl₂ (310 mg, 2.60 mmol, 189 μL, 4.0 eq) under a N₂ atmosphere. The mixture was stirred at 20° C. for 1 h. LCMS (the sample was quenched with MeOH) indicated that the starting material was completely consumed, and the desired mass was detected. The reaction mixture was concentrated under vacuum to give methyl 3-(3-chloro-3-oxopropyl)-5-methyl-1H-indole-6-carboxylate (180 mg) as a brown solid, which was used in the next step without any further purification. M+H⁺=276.2 (LCMS).

### Step 3: Methyl 7-methyl-3-oxo-1,2,3,4-tetrahydrocyclopenta[b]indole-6-carboxylate (169A-3)

To a solution of AlCl₃ (343 mg, 2.57 mmol, 141 μL, 4.0 eq) in DCE (10 mL) was added methyl 3-(3-chloro-3-oxopropyl)-5-methyl-1H-indole-6-carboxylate (180 mg, 644 μmol, 1.0 eq). The mixture was stirred at 20° C. for 14 h. LCMS indicated that the starting material was completely consumed, and the desired mass was detected. The reaction mixture was poured into saturated aqueous NaHCO₃ (20 mL) and extracted with DCM (10 mL×3). The combined organic layers were dried over Na₂SO₄, filtered, and concentrated under vacuum to give a residue which was purified by preparative TLC (EtOAc/petroleum ether=1/1, R_{f}=0.6). Methyl 7-methyl-3-oxo-1,2,3,4-tetrahydrocyclopenta[b]indole-6-carboxylate (50.0 mg) was obtained as a crude brown solid. M+H⁺=244.2 (LCMS); ¹H NMR (400 MHZ, DMSO-d₆) δ 11.86 (s, 1H), 7.96 (s, 1H), 7.65 (s, 1H), 3.85 (s, 3H), 3.06-3.01 (m, 2H), 2.96-2.90 (m, 2H), 2.58 (s, 3H).

### Step 4: 7-Methyl-3-oxo-1,2,3,4-tetrahydrocyclopenta[b]indole-6-carboxylic acid (169A-4)

To a solution of 7-methyl-3-oxo-1,2,3,4-tetrahydrocyclopenta[b]indole-6-carboxylate (40.0 mg, 164 μmol, 1.0 eq) in a mixture of THF (3.0 mL) and H₂O (1.0 mL) was added NaOH (32.9 mg, 822 μmol, 5.0 eq). The mixture was stirred at 80° C. for 5 h. LCMS indicated that the starting material was completely consumed, and the desired mass was detected. The reaction mixture was allowed to cool to room temperature, treated with H₂O (5.0 mL) and washed with MTBE (5.0 mL×2). The aqueous layer was acidified to pH 5 with HCl (1 M aqueous). The product was extracted with EtOAc (5.0 mL×3). The combined organic layers were dried over Na₂SO₄, filtered, and concentrated under vacuum to give the crude product 7-methyl-3-oxo-1,2,3,4-tetrahydrocyclopenta[b]indole-6-carboxylic acid (37.0 mg), which was used in the next step without any further purification. M−H⁻=227.8 (LCMS).

### Step 5: 7-Methyl-N-(1-(naphthalen-1-yl)cyclopropyl)-3-oxo-1,2,3,4-tetrahydrocyclopenta[b]indole-6-carboxamide (Compound 334)

To a solution of 7-methyl-3-oxo-1,2,3,4-tetrahydrocyclopenta[b]indole-6-carboxylic acid (30.0 mg, 130 μmol, 1.0 eq) and 1-(naphthalen-1-yl)cyclopropanamine (24.0 mg, 131 μmol, 1.0 eq) in DCM (3.0 mL) were added TEA (40.0 mg, 393 μmol, 54.7 μL, 3.0 eq), EDCI (62.7 mg, 327 μmol, 2.5 eq) and HOBt (44.2 mg, 327 μmol, 2.5 eq). The mixture was stirred at 20° C. for 1 h. LCMS indicated that the starting material was completely consumed, and the desired mass was detected. The reaction mixture was poured into H₂O (5.0 mL) and extracted with DCM (5.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 30%-65% B over 8 min; mobile phase A: 0.2% aqueous FA, mobile phase B: acetonitrile). 7-Methyl-N-(1-(naphthalen-1-yl)cyclopropyl)-3-oxo-1,2,3,4-tetrahydrocyclopenta[b]indole-6-carboxamide (20.0 mg, 49.5 μmol, 38% yield) was obtained as a white solid. M+H⁺=395.0 (LCMS); ¹H NMR (400 MHZ, DMSO-d₆) δ 1.57 (s, 1H), 9.22 (s, 1H), 8.69 (br d, J=7.8 Hz, 1H), 7.95 (br d, J=7.6 Hz, 1H), 7.84 (br d, J=7.6 Hz, 2H), 7.68-7.45 (m, 4H), 7.11 (s, 1H), 2.97 (br s, 2H), 2.86 (br s, 2H), 2.14 (s, 3H), 1.38 (br s, 2H), 1.19 (br s, 2H).

### Example 170: 7-Methyl-N-(1-(naphthalen-1-yl)cyclopropyl)-3-oxo-2,3,9,9a-tetrahydro-1H-imidazo[1,5-a]indole-6-carboxamide (Compound 194)

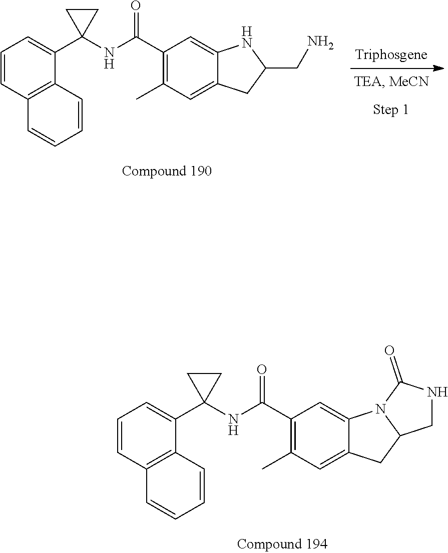 [SMILES: Cc1cc2c(cc1C(=O)NC1(c3cccc4ccccc34)CC1)NC(CN)C2 ; Cc1cc2c(cc1C(=O)NC1(c3cccc4ccccc34)CC1)N1C(=O)NCC1C2]

### Step 1: 7-Methyl-N-(1-(naphthalen-1-yl)cyclopropyl)-3-oxo-2,3,9,9a-tetrahydro-1H-imidazo[1,5-a]indole-6-carboxamide (Compound 194)

To a solution of 2-(aminomethyl)-5-methyl-N-(1-(naphthalen-1-yl)cyclopropyl)indoline-6-carboxamide (90.0 mg, 185 μmol, 1.0 eq, TFA salt) and TEA (467 mg, 4.62 mmol, 643 μL, 25 eq) in acetonitrile (12 mL) at 0° C. was added a solution of triphosgene (55.0 mg, 185 μmol, 1.0 eq) in acetonitrile (3.0 mL) over 5 min under a N₂ atmosphere. The mixture was stirred at the same temperature for another 20 min. LCMS indicated that the starting material was completely consumed, and the desired mass was detected. The reaction mixture was poured into aqueous NaOH (0.05 M, 60 mL) and extracted with DCM (30 mL×3). The combined organic layers were dried over Na₂SO₄, filtered, and concentrated under vacuum to give a residue which was purified by preparative HPLC (Phenomenex luna C18 column (80×40 mm, 3 μm); flow rate: 25 mL/min; gradient: 40%-60% B over 7 min; mobile phase A: 0.04% aqueous HCl, mobile phase B: acetonitrile). 7-Methyl-N-(1-(naphthalen-1-yl)cyclopropyl)-3-oxo-2,3,9,9a-tetrahydro-1H-imidazo[1,5-a]indole-6-carboxamide (14.6 mg, 33.3 μmol, 18% yield) was obtained as a white solid. M+H⁺=398.0 (LCMS); ¹H NMR (400 MHZ, DMSO-d₆) δ 9.08 (s, 1H), 8.65 (d, J=8.3 Hz, 1H), 7.93 (d, J=7.7 Hz, 1H), 7.82 (dd, J=2.4, 7.8 Hz, 2H), 7.60-7.42 (m, 3H), 7.02 (s, 1H), 6.97 (s, 1H), 6.89 (s, 1H), 4.57 (dd, J=6.8, 8.9 Hz, 1H), 3.67 (t, J=9.2 Hz, 1H), 3.27 (br dd, J=6.9, 9.1 Hz, 1H), 3.16 (dd, J=9.2, 16.4 Hz, 1H), 2.92 (dd, J=8.9, 16.5 Hz, 1H), 1.95 (s, 3H), 1.41-1.30 (m, 2H), 1.26-1.07 (m, 2H).

### Example 171: 5-(2-(Dimethylamino)ethoxy)-N-(1-(2-fluorophenyl)cyclopropyl)-2-methylbenzamide (Compound 272)

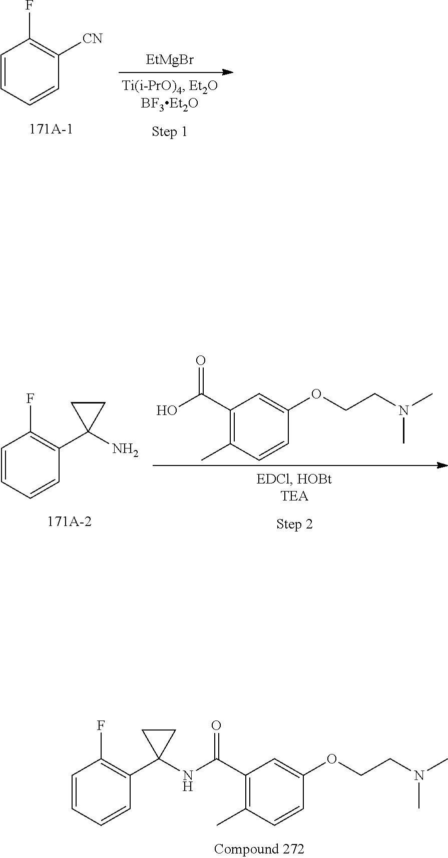 [SMILES: "EtMgBr" ; N#Cc1ccccc1F ; NC1(c2ccccc2F)CC1 ; Cc1ccc(OCCN(C)C)cc1C(=O)O ; Cc1ccc(OCCN(C)C)cc1C(=O)NC1(c2ccccc2F)CC1]

### Step 1: 1-(2-Fluorophenyl)cyclopropanamine (171A-2)

A solution of 2-fluorobenzonitrile (1.00 g, 8.26 mmol, 1.0 eq) in anhydrous Et₂O (50 mL) was degassed and purged with N₂ three times. To this mixture was added Ti(i-PrO)₄ (2.35 g, 8.26 mmol, 2.44 mL, 1.0 eq) slowly at −78° C., and then EtMgBr (3 M in Et₂O, 6.06 mL, 2.2 eq) was added dropwise to maintain the temperature between −78° C. and −75° C. over 1 h under a N₂ atmosphere. After the addition was complete, the resulting mixture was stirred at the same temperature for 10 min and warmed to 20° C. over 1 h. BF₃·Et₂O (2.34 g, 16.5 mmol, 2.04 mL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (30 mL) and MTBE (10 mL), and extracted with MTBE (30 mL×4). The aqueous layer was basified to pH 8 using NaOH (2 M aqueous), and a precipitate was formed. The mixture was filtered through a pad of Celite and the slurry was washed with DCM several times. The combined filtrate was extracted with DCM (50 mL×4). The combined organic layers were dried over Na₂SO₄, filtered, and concentrated under vacuum to give a residue which was purified by flash silica gel chromatography using a gradient of EtOAc/petroleum ether from 0/1 to 1/1. 1-(2-Fluorophenyl)cyclopropanamine (400 mg, 2.65 mmol, 32% yield) was obtained as a colorless oil. M+H⁺=152.1 (LCMS).

### Step 2: 5-(2-(Dimethylamino)ethoxy)-N-(1-(2-fluorophenyl)cyclopropyl)-2-methyl benzamide (Compound 272)

To a solution of 1-(2-fluorophenyl)cyclopropanamine (100 mg, 483 μmol, 1.0 eq) and 5-(2-(dimethylamino)ethoxy)-2-methylbenzoic acid (108 mg, 483 μmol, 1.0 eq) in DCM (2.0 mL) were added TEA (147 mg, 1.45 mmol, 202 μL, 3.0 eq), EDCI (139 mg, 724 μmol, 1.5 eq) and HOBt (78.0 mg, 724 μmol, 1.5 eq). The mixture was stirred at 25° C. for 16 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 10%-30% B over 8 min; mobile phase A: 0.04% aqueous HCl, mobile phase B: acetonitrile). 5-(2-(Dimethylamino)ethoxy)-N-(1-(2-fluorophenyl)cyclopropyl)-2-methyl benzamide (109 mg, 288 μmol, 60% yield) was obtained as a white solid. M+H⁺=357.1 (LCMS); ¹H NMR (400 MHz, DMSO-d₆) δ 10.23-10.10 (m, 1H), 8.98 (s, 1H), 7.62-7.55 (m, 1H), 7.32-7.24 (m, 1H), 7.17-7.09 (m, 3H), 6.94 (dd, J=2.7, 8.3 Hz, 1H), 6.83 (d, J=2.8 Hz, 1H), 4.30 (t, J=4.9 Hz, 2H), 3.47 (q, J=5.2 Hz, 2H), 2.82 (d, J=4.9 Hz, 6H), 2.12 (s, 3H), 1.23-1.17 (m, 2H), 1.17-1.13 (m, 2H).

### Example 172: 5-(2-(Dimethylamino)ethoxy)-N-(1-(3-fluorophenyl)cyclopropyl)-2-methylbenzamide (Compound 252)

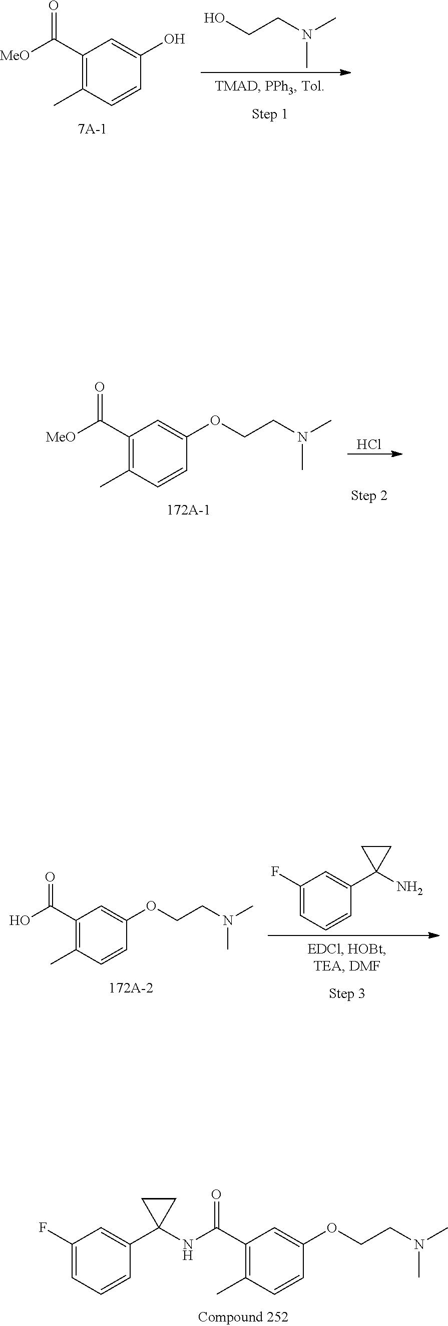

### Step 1: Methyl 5-(2-(dimethylamino)ethoxy)-2-methylbenzoate (172A-1)

A mixture of methyl 5-hydroxy-2-methylbenzoate (5.00 g, 30.1 mmol, 1.0 eq), 2-(dimethylamino) ethanol (2.68 g, 30.1 mmol, 3.02 mL, 1.0 eq), TMAD (10.4 g, 60.2 mmol, 2.0 eq) and PPh₃ (15.8 g, 60.2 mmol, 2.0 eq) in toluene (150 mL) was degassed and purged with N₂ three times. The mixture was stirred at 100° C. for 16 h under a N₂ atmosphere. LCMS indicated that the starting material completely consumed, and the desired mass was detected. The reaction mixture was allowed to cool to room temperature, poured into H₂O (20 mL) and extracted with EtOAc (20 mL×3). The combined EtOAc layers were dried over Na₂SO₄, filtered, and concentrated under vacuum to give a residue which was purified by flash silica gel chromatography using a gradient of EtOAc/petroleum ether from 0/1 to 1/10. Methyl 5-(2-(dimethylamino)ethoxy)-2-methylbenzoate (6.00 g, 25.3 mmol, 84% yield) was obtained as a white solid. ¹H NMR (400 MHz, CD₃OD) δ 7.43 (d, J=2.6 Hz, 1H), 7.18 (d, J=8.5 Hz, 1H), 7.03 (dd, J=2.8, 8.4 Hz, 1H), 4.18-4.03 (m, 2H), 3.87 (s, 3H), 2.76 (t, J=5.4 Hz, 2H), 2.47 (s, 3H), 2.34 (s, 6H).

### Step 2: 5-(2-(Dimethylamino)ethoxy)-2-methylbenzoic acid (172A-2)

A solution of methyl 5-(2-(dimethylamino)ethoxy)-2-methylbenzoate (4.00 g, 16.8 mmol, 1.0 eq) in HCl (2 M aqueous, 20 mL) was stirred at 110° C. for 16 h. LCMS indicated that the starting material completely consumed, and the desired product was detected. The reaction mixture was allowed to cool to room temperature, basified to pH 6 using NaOH (2 M aqueous) the mixture was concentrated under vacuum to give the crude product 5-(2-(dimethylamino)ethoxy)-2-methylbenzoic acid (2.00 g, HCl salt) as a white solid.

### Step 3: 5-(2-(Dimethylamino)ethoxy)-N-(1-(3-fluorophenyl)cyclopropyl)-2-methylbenzamide (Compound 252)

To a stirred solution of 5-(2-(dimethylamino)ethoxy)-2-methylbenzoic acid (50.0 mg, 224 μmol, 1.0 eq) and 1-(3-fluorophenyl)cyclopropanamine (33.9 mg, 224 μmol, 1.0 eq) in DMF (1.0 mL) were added EDCI (64.4 mg, 336 μmol, 1.5 eq), HOBt (45.4 mg, 336 μmol, 1.5 eq) and TEA (68.0 mg, 672 μmol, 3.0 eq). The mixture was stirred at 25° C. for 1 h. LCMS indicated that the starting material was completely consumed, and the desired mass was detected. The reaction mixture was poured into water (10 mL) and extracted with EtOAc (10 mL×3). The combined organic layers were washed with brine (10 mL×3), dried over Na₂SO₄, filtered, and concentrated under vacuum to give a residue which was purified by preparative HPLC (Phenomenex Luna column (80×30 mm, 3 μm); flow rate: 25 mL/min; gradient: 15%-50% B over 8 min; mobile phase A: 0.04% aqueous HCl, mobile phase B: acetonitrile) to give 5-(2-(dimethylamino)ethoxy)-N-(1-(3-fluorophenyl)cyclopropyl)-2-methylbenzamide (8.60 mg, 24.1 μmol, 11% yield, HCl salt) as a white solid. M+H⁺=357.1 (LCMS); ¹H NMR (400 MHz, CDCl₃) δ 12.90-12.56 (m, 1H), 7.17-7.04 (m, 5H), 6.94-6.86 (m, 2H), 4.55 (br d, J=1.1 Hz, 2H), 3.55-3.36 (m, 2H), 2.92 (br s, 6H), 2.36 (s, 3H), 1.48-1.35 (m, 4H).

### Example 173: 5-(2-(Dimethylamino)ethoxy)-N-(1-(4-fluorophenyl)cyclopropyl)-2-methylbenzamide (Compound 259)

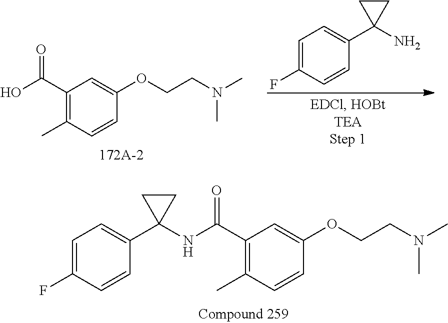

### Step 1: 5-(2-(Dimethylamino)ethoxy)-N-(1-(4-fluorophenyl)cyclopropyl)-2-methyl benzamide (Compound 259)

To a solution of 1-(4-fluorophenyl)cyclopropanamine (100 mg, 661 μmol, 1.2 eq) and 5-(2-((tert-butoxycarbonyl)amino)ethoxy)-2-methylbenzoic acid (123 mg, 551 μmol, 1.0 eq) in DCM (1.0 mL) were added TEA (167 mg, 1.65 mmol, 230 μL, 3.0 eq), EDCI (159 mg, 827 μmol, 1.5 eq) and HOBt (112 mg, 827 μmol, 1.5 eq). The mixture was stirred at 25° C. for 3 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 1%-40% B over 8 min; mobile phase A: 0.04% aqueous HCl, mobile phase B: acetonitrile). 5-(2-(Dimethylamino)ethoxy)-N-(1-(4-fluorophenyl)cyclopropyl)-2-methyl benzamide (118 mg, 326 μmol, 59% yield) was obtained as a white solid. M+H⁺=357.1 (LCMS); ¹H NMR (400 MHZ, CDCl₃) δ 12.97-12.63 (m, 1H), 7.46-7.38 (m, 2H), 7.11 (d, J=8.4 Hz, 1H), 7.04-6.96 (m, 3H), 6.90 (s, 1H), 6.85 (dd, J=2.6, 8.4 Hz, 1H), 4.57-4.49 (m, 2H), 3.47-3.38 (m, 2H), 2.93 (d, J=4.8 Hz, 6H), 2.33 (s, 3H), 1.42-1.35 (m, 2H), 1.34-1.28 (m, 2H).

### Example 174: N-(1-(2-Bromophenyl)cyclopropyl)-5-(2-(dimethylamino)ethoxy)-2-methylbenzamide (Compound 251)

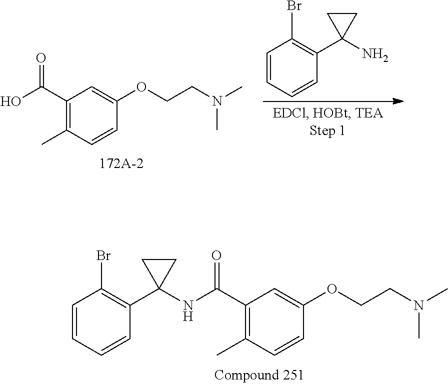

### Step 1: N-(1-(2-Bromophenyl)cyclopropyl)-5-(2-(dimethylamino)ethoxy)-2-methyl benzamide (Compound 251)

To a solution of 5-(2-(dimethylamino)ethoxy)-2-methylbenzoic acid (50.0 mg, 224 μmol, 1.0 eq) and 1-(2-bromophenyl)cyclopropanamine (47.5 mg, 224 μmol, 1.0 eq) in DMF (5.0 mL) were added TEA (45.3 mg, 448 μmol, 62.3 μL, 2.0 eq), EDCI (51.5 mg, 267 μmol, 1.2 eq) and HOBt (36.3 mg, 269 μmol, 1.2 eq). The mixture was stirred at 25° C. for 16 h. LCMS indicated that the starting material was completely consumed, and the desired mass was detected. The reaction mixture was poured into H₂O (10 mL) and extracted with DCM (5.0 mL×5). The combined organic layers were dried over Na₂SO₄, filtered, and concentrated under vacuum to give a residue which was purified by preparative HPLC (Phenomenex C18 column (75×30 mm, 3 μm); flow rate: 25 mL/min; gradient: 25%-65% B over 8 min; mobile phase A: 10 mM aqueous NH₄HCO₃, mobile phase B: acetonitrile). N-(1-(2-Bromophenyl)cyclopropyl)-5-(2-(dimethylamino)ethoxy)-2-methylbenzamide (20.2 mg, 48.1 μmol, 21% yield) was obtained as a yellow gum. M+H⁺=417.0 (LCMS); ¹H NMR (400 MHZ, CDCl₃) δ 7.80 (dd, J=1.6, 7.6 Hz, 1H), 7.55 (dd, J=0.9, 7.9 Hz, 1H), 7.31 (dt, J=1.1, 7.5 Hz, 1H), 7.15 (dt, J=1.6, 7.7 Hz, 1H), 7.06 (d, J=8.1 Hz, 1H), 6.89-6.82 (m, 2H), 6.78 (s, 1H), 4.04 (t, J=5.6 Hz, 2H), 2.73 (t, J=5.6 Hz, 2H), 2.35 (s, 6H), 2.24 (s, 3H), 1.32 (dd, J=4.3, 14.1 Hz, 4H).

### Example 175: N-(1-(3-Bromophenyl)cyclopropyl)-5-(2-(dimethylamino)ethoxy)-2-methylbenzamide (Compound 261)

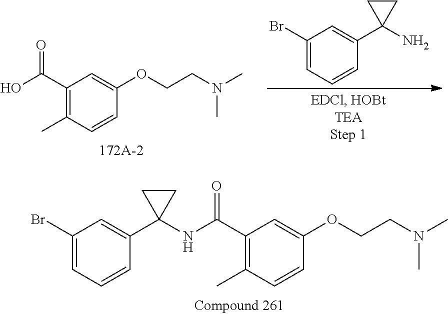 [SMILES: Cc1ccc(OCCN(C)C)cc1C(=O)O ; NC1(c2cccc(Br)c2)CC1 ; Cc1ccc(OCCN(C)C)cc1C(=O)NC1(c2cccc(Br)c2)CC1]

### Step 1: N-(1-(3-Bromophenyl)cyclopropyl)-5-(2-(dimethylamino)ethoxy)-2-methylbenzamide (Compound 261)

A mixture of 5-(2-(dimethylamino)ethoxy)-2-methylbenzoic acid (351 mg, 472 μmol, 2.0 eq), 1-(3-bromophenyl)cyclopropanamine (50.0 mg, 236 μmol, 1.0 eq), TEA (71.6 mg, 707 μmol, 98.4 μL, 3.0 eq), HOBt (47.8 mg, 354 μmol, 1.5 eq) and EDCI (67.8 mg, 354 μmol, 1.5 eq) in DCM (3.0 mL) was stirred at 20° C. for 16 h. LCMS indicated that the starting material was completely consumed, and the desired mass was detected. The reaction mixture was poured into H₂O (4.0 mL) and extracted with EtOAc (3.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 5%-45% B over 8 min; mobile phase A: 0.04% aqueous HCl, mobile phase B: acetonitrile). N-(1-(3-bromophenyl)cyclopropyl)-5-(2-(dimethylamino)ethoxy)-2-methylbenzamide (41.6 mg, 100 μmol, 42% yield, HCl salt) was obtained as a pink solid. M+H⁺=417.0 (LCMS); ¹H NMR (400 MHZ, DMSO-d₆) δ 10.56-10.42 (m, 1H), 9.09-9.04 (m, 1H), 7.43-7.35 (m, 2H), 7.29-7.24 (m, 1H), 7.22-7.17 (m, 2H), 7.01-6.96 (m, 2H), 4.39-4.33 (m, 2H), 3.54-3.44 (m, 2H), 2.85-2.81 (m, 6H), 2.28-2.22 (m, 3H), 1.28 (br d, J=5.0 Hz, 4H).

### Example 176: N-(1-(4-Bromophenyl)cyclopropyl)-5-(2-(dimethylamino)ethoxy)-2-methylbenzamide (Compound 258)

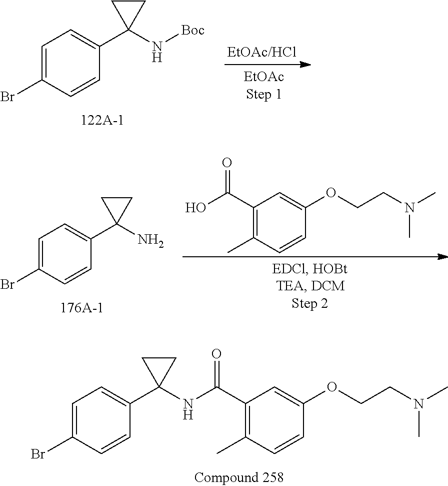 [SMILES: CC(C)(C)OC(=O)NC1(c2ccc(Br)cc2)CC1 ; NC1(c2ccc(Br)cc2)CC1 ; Cc1ccc(OCCN(C)C)cc1C(=O)O ; Cc1ccc(OCCN(C)C)cc1C(=O)NC1(c2ccc(Br)cc2)CC1]

### Step 1: 1-(4-Bromophenyl)cyclopropanamine (176A-1)

To a stirred solution of tert-butyl(1-(4-bromophenyl)cyclopropyl)carbamate (300 mg, 961 μmol, 1.0 eq) in EtOAc (6.0 mL) was added HCl/EtOAc (4 M, 5.0 mL). The mixture was stirred at 20° C. for 2 h. TLC indicated that the starting material was completely consumed. The mixture was concentrated under vacuum at 30° C. to give 1-(4-bromophenyl)cyclopropanamine (230 mg, 932 μmol, 97% yield, HCl salt) as a white solid.

### Step 2: N-(1-(4-Bromophenyl)cyclopropyl)-5-(2-(dimethylamino)ethoxy)-2-methylbenzamide (Compound 258)

To a solution of 1-(4-bromophenyl)cyclopropanamine (80.0 mg, 322 μmol, 1.0 eq) and 5-(2-(dimethylamino)ethoxy)-2-methylbenzoic acid (71.9 mg, 322 μmol, 1.0 eq) in DCM (3.0 mL) were added TEA (97.7 mg, 966 μmol, 134 μL, 3.0 eq), EDCI (154 mg, 805 μmol, 2.5 eq) and HOBt (109 mg, 805 μmol, 2.5 eq). The mixture was stirred at 20° C. for 2 h. LCMS indicated that the starting material was completely consumed, and the desired mass was detected. The reaction mixture was poured into H₂O (3.0 mL) and extracted with DCM (3.0 mL×2). The combined organic layers were dried over Na₂SO₄, filtered, and concentrated under vacuum to give a residue which was purified by preparative HPLC (Phenomenex Gemini C18 column (75× 30 mm, 3 μm); flow rate: 25 mL/min; gradient: 20%-60% B over 8 min; mobile phase A: 10 mM aqueous NH₄HCO₃, mobile phase B: acetonitrile). N-(1-(4-Bromophenyl)cyclopropyl)-5-(2-(dimethyl amino)ethoxy)-2-methylbenzamide (12.8 mg, 27.9 μmol, 9% yield) was obtained as a yellow gum. M+H⁺=417.0 (LCMS); ¹H NMR (400 MHZ, DMSO-d₆) δ 8.97 (s, 1H), 7.48 (s, 2H), 7.18 (d, J=8.5 Hz, 3H), 6.93 (s, 2H), 4.05 (t, J=5.8 Hz, 2H), 2.61 (t, J=5.8 Hz, 2H), 2.36-2.17 (m, 9H), 1.25 (br d, J=5.5 Hz, 4H)

### Example 177: N-(1-(2-chlorophenyl)cyclopropyl)-5-(2-(dimethylamino)ethoxy)-2-methylbenzamide (Compound 326)

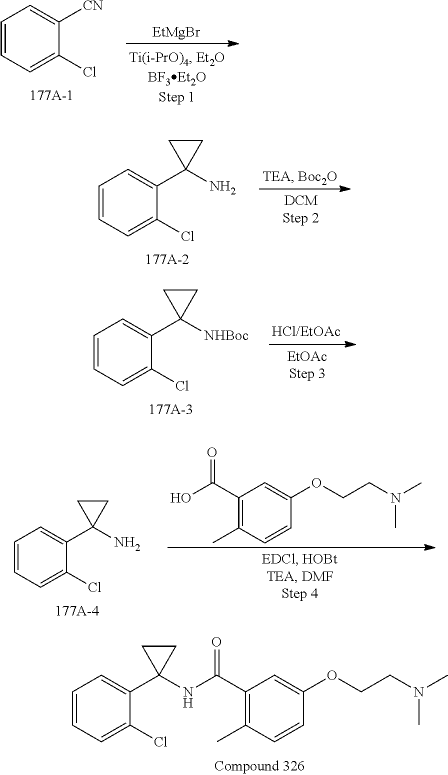

### Step 1: 1-(2-Chlorophenyl)cyclopropanamine (177A-2)

A mixture of 2-chlorobenzonitrile (500 mg, 2.39 mmol, 1.0 eq) in anhydrous Et₂O (35 mL) was degassed and purged with N₂ three times. The mixture was stirred at −78° C. To this mixture was added Ti(i-PrO)₄ (1.14 g, 4.00 mmol, 1.18 mL, 1.1 eq) slowly, and then EtMgBr (3 M in Et₂O, 2.67 mL, 2.2 eq) was added dropwise to maintain the temperature between-78° C. and −75° C. over 5 min under a N₂ atmosphere. After the addition was complete, the resulting mixture was stirred at the same temperature for 10 min and warmed to 20° C. over 1 h. BF₃·Et₂O (1.03 g, 7.27 mmol, 897 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (30 mL) and MTBE (30 mL), and extracted with MTBE (30 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (20 mL×4). The combined organic layers were dried over Na₂SO₄, filtered, and concentrated under vacuum to give crude product 1-(2-chlorophenyl)cyclopropanamine (600 mg) as a brown oil. M+H⁺=168.1 (LCMS).

### Step 2: tert-Butyl(1-(2-Chlorophenyl)cyclopropyl)carbamate (177A-3)

To a solution of 1-(2-chlorophenyl)cyclopropanamine (600 mg, 3.58 mmol, 1.0 eq) in DCM (30 mL) were added TEA (724 mg, 7.16 mmol, 996 μL, 2.0 eq) and Boc₂O (937 mg, 4.30 mmol, 987 μL, 1.2 eq). The mixture was stirred at 20° C. for 16 h. LCMS indicated that the starting material was completely consumed, and the desired mass was detected. The reaction mixture was poured into H₂O (30 mL) and extracted with DCM (30 mL×2). The combined organic layers were dried over Na₂SO₄, filtered, and concentrated under vacuum to give the crude product tert-butyl(1-(2-chlorophenyl)cyclopropyl)carbamate (360 mg) as a white solid. M−56+H⁺=212.0 (LCMS).

### Step 3: 1-(2-Chlorophenyl)cyclopropanamine (177A-4)

To a stirred solution of tert-butyl(1-(2-chlorophenyl)cyclopropyl)carbamate (360 mg, 1.34 mmol, 1.0 eq) in EtOAc (3.0 mL) was added HCl/EtOAc (4 M, 15 mL). The mixture was stirred at 25° C. for 1 h. LCMS indicated that the starting material was completely consumed, and the desired mass was detected. The mixture was concentrated under vacuum at 30° C. to give product 1-(2-chlorophenyl)cyclopropanamine (260 mg, HCl salt) as a brown solid. M+H⁺=168.0 (LCMS).

### Step 4: N-(1-(2-Chlorophenyl)cyclopropyl)-5-(2-(dimethylamino)ethoxy)-2-methylbenzamide (Compound 326)

To a solution of 1-(2-chlorophenyl)cyclopropanamine (50.0 mg, 298 μmol, 1.0 eq) and 5-(2-(dimethylamino)ethoxy)-2-methylbenzoic acid (133 mg, 596 μmol, 2.0 eq) in DMF (2.0 mL) were added TEA (90.5 mg, 895 μmol, 174 μL, 3.0 eq), EDCI (68.6 mg, 501 μmol, 1.2 eq) and HOBt (48.4 mg, 358 μmol, 1.2 eq). The mixture was stirred at 25° C. for 3 h. LCMS indicated that the starting material was completely consumed, and the desired mass was detected. The reaction mixture was poured into H₂O (4.0 mL) and extracted with EtOAc (2.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 30%-70% B over 8 min; mobile phase A: 0.2% aqueous FA, mobile phase B: acetonitrile). N-(1-(2-Chlorophenyl)cyclopropyl)-5-(2-(dimethylamino)ethoxy)-2-methylbenzamide (37.7 mg, 90.6 μmol, 30% yield, FA salt) was obtained as a white solid. M+H⁺=373.0 (LCMS); ¹H NMR (400 MHZ, CDCl₃) δ 10.56 (br s, 1H), 8.39 (s, 1H), 7.79 (dd, J=1.9, 7.4 Hz, 1H), 7.36 (dd, J=1.4, 7.5 Hz, 1H), 7.25-7.18 (m, 1H), 7.06 (d, J=8.1 Hz, 1H), 6.93-6.68 (m, 3H), 4.21 (t, J=5.1 Hz, 2H), 3.13 (t, J=5.0 Hz, 2H), 2.62 (s, 6H), 2.22 (s, 3H), 1.32 (d, J=4.4 Hz, 2H), 1.28 (d, J=4.4 Hz, 2H).

### Example 178: 5-(2-(Dimethylamino)ethoxy)-2-methyl-N-(1-(0-tolyl)cyclopropyl)benzamide (Compound 282)

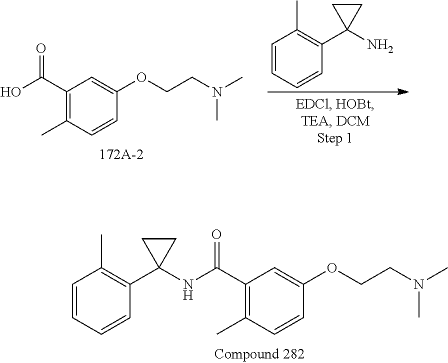

### Step 1: 5-(2-(Dimethylamino)ethoxy)-2-methyl-N-(1-(o-tolyl)cyclopropyl)benzamide (Compound 282)

To a mixture of 1-(o-tolyl)cyclopropanamine (100 mg, 679 μmol, 1.1 eq) and 5-(2-(dimethylamino)ethoxy)-2-methylbenzoic acid (138 mg, 618 μmol, 1.0 eq) in DCM (2.0 mL) were added EDCI (178 mg, 926 μmol, 1.5 eq), HOBt (125 mg, 926 μmol, 1.5 eq) and TEA (187 mg, 1.85 mmol, 3.0 eq) at 25° C. The mixture was stirred at 25° C. for 16 h. LCMS indicated that the starting material was completely consumed, and the desired mass was detected. The reaction mixture was poured into water (10 mL) and extracted with EtOAc (10 mL×3). The combined organic layers were washed with brine (10 mL×3), dried over Na₂SO₄, filtered, and concentrated under vacuum to give a residue which was purified by preparative HPLC (Phenomenex Luna column (80×30 mm, 3 μm); flow rate: 25 mL/min; gradient: 10%-40% B over 8 min; mobile phase A: 0.04% aqueous HCl, mobile phase B: acetonitrile). 5-(2-(Dimethylamino)ethoxy)-2-methyl-N-(1-(o-tolyl)cyclopropyl)benzamide (32.4 mg, 91.9 μmol, 15% yield, HCl salt) was obtained as a yellow gum. M+H⁺=353.1 (LCMS); ¹H NMR (400 MHz, DMSO-d₆) δ 10.51 (br s, 1H), 8.90 (s, 1H), 7.59 (br d, J=5.9 Hz, 1H), 7.15-7.08 (m, 4H), 6.94-6.89 (m, 1H), 6.75 (d, J=2.6 Hz, 1H), 4.30 (br t, J=5.0 Hz, 2H), 3.49-3.42 (m, 2H), 2.80 (d, J=4.9 Hz, 6H), 2.48 (s, 3H), 2.07 (s, 3H), 1.18-1.11 (m, 2H), 1.07-0.99 (m, 2H).

### Example 179: 5-(2-(Dimethylamino)ethoxy)-2-methyl-N-(1-(m-tolyl)cyclopropyl)benzamide (Compound 255)

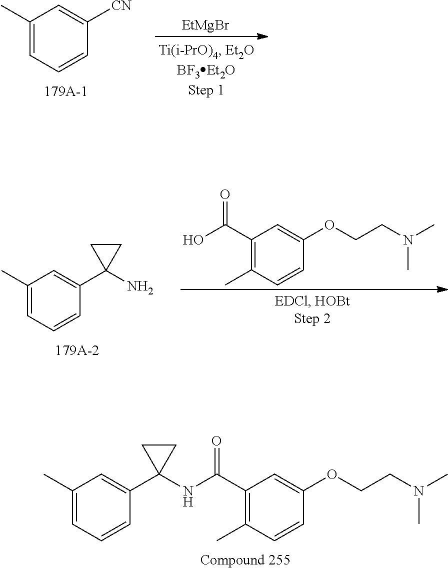

### Step 1: 1-(m-Tolyl)cyclopropanamine (179A-2)

A mixture of 3-methylbenzonitrile (1.00 g, 8.54 mmol, 1.02 mL, 1.0 eq) in anhydrous Et₂O (40 mL) was degassed and purged with N₂ three times. The mixture was cooled to −78° C. under a N₂ atmosphere. To this mixture was added Ti(i-PrO)₄ (2.67 g, 9.39 mmol, 2.77 mL, 1.1 eq) slowly, and then EtMgBr (3 M in Et₂O, 6.26 mL, 2.2 eq) was added dropwise over 1 h to maintain the temperature between −78° C. and −73° C. under a N₂ atmosphere. After the addition was complete, the resulting mixture was stirred at the same temperature for 10 min and warmed to 20° C. over 1 h. BF₃·Et₂O (2.42 g, 17.1 mmol, 2.11 mL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (40 mL) and MTBE (10 mL), and extracted with MTBE (40 mL×4). The aqueous layer was basified to pH 8 using NaOH (2 M aqueous), and a precipitate was formed. The mixture was filtered through a pad of Celite and the slurry was washed with DCM several times. The combined filtrate was extracted with DCM (20 mL×4). The combined organic layers were dried over Na₂SO₄, filtered, and concentrated under vacuum to give a residue which was purified by flash silica gel chromatography using a gradient of EtOAc/petroleum ether from 0/1 to 1/0. 1-(m-Tolyl)cyclopropanamine (200 mg, 1.36 mmol, 16% yield) was obtained as a yellow oil. M+H⁺=148.1 (LCMS).

### Step 2: 5-(2-(Dimethylamino)ethoxy)-2-methyl-N-(1-(m-tolyl)cyclopropyl)benzamide (Compound 255)

To a solution of 1-(m-tolyl)cyclopropanamine (70.0 mg, 475 μmol, 1.0 eq) and 5-(2-(dimethyl amino)ethoxy)-2-methylbenzoic acid (106 mg, 475 μmol, 1.0 eq) in DMF (1.0 mL) were added TEA (96.2 mg, 951 μmol, 132 μL, 2.0 eq), EDCI (109 mg, 571 μmol, 1.2 eq) and HOBt (77.1 mg, 571 μmol, 1.2 eq). The mixture was stirred at 25° C. for 16 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by preparative TLC (DCM/ethanol=10/1, R_{f}=0.2). 5-(2-(Dimethylamino)ethoxy)-2-methyl-N-(1-(m-tolyl)cyclopropyl)benzamide (70.8 mg, 201 μmol, 42% yield) was obtained as a white solid. M+H⁺=353.1 (LCMS); ¹H NMR (400 MHZ, CDCl₃) δ 7.24-7.18 (m, 1H), 7.17-7.07 (m, 3H), 7.03 (d, J=7.4 Hz, 1H), 6.97 (d, J=2.6 Hz, 1H), 6.88 (dd, J=2.8, 8.4 Hz, 1H), 6.41 (s, 1H), 4.10 (t, J=5.5 Hz, 2H), 2.79 (t, J=5.4 Hz, 2H), 2.40 (s, 6H), 2.36 (s, 3H), 2.35 (s, 3H), 1.36 (s, 4H).

### Example 180: 5-(2-(Dimethylamino)ethoxy)-2-methyl-N-(1-(p-tolyl)cyclopropyl)benzamide (Compound 253)

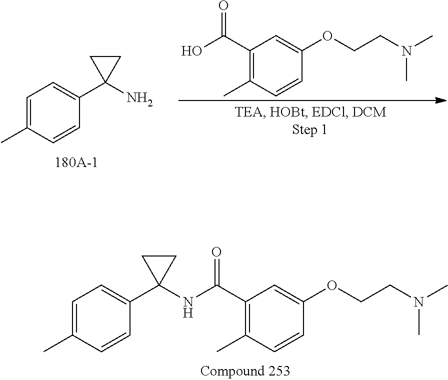

### Step 1: 5-(2-(Dimethylamino)ethoxy)-2-methyl-N-(1-(p-tolyl)cyclopropyl)benzamide (Compound 253)

To a solution of 1-(p-tolyl)cyclopropanamine (69.2 mg, 470 μmol, 1.0 eq) in DCM (8.0 mL) were added 5-(2-(dimethylamino)ethoxy)-2-methylbenzoic acid (100 mg, 448 μmol, 1.0 eq), TEA (136 mg, 1.34 mmol, 187 μL, 3.0 eq), EDCI (129 mg, 672 μmol, 1.5 eq) and HOBt (90.8 mg, 672 μmol, 1.5 eq). The mixture was stirred at 20° C. for 16 h. LCMS indicated that the starting material completely consumed, and the desired product was detected. The reaction mixture was poured into H₂O (10 mL) and extracted with DCM (10 mL×5). The combined layers were dried over Na₂SO₄, filtered, and concentrated under vacuum to give a residue which was purified by preparative HPLC (Phenomenex Luna C18 column (80×40 mm, 3 μm); flow rate: 25 mL/min; gradient: 15%-45% B over 8 min; mobile phase A: 10 mM aqueous NH₄HCO₃, mobile phase B: acetonitrile). 5-(2-(Dimethylamino)ethoxy)-2-methyl-N-(1-(p-tolyl)cyclopropyl)benzam ide (68.8 mg, 194 μmol, 43% yield) was obtained as a white solid. M+H⁺=353.1 (LCMS); ¹H NMR (400 MHZ, DMSO-d₆) δ 8.91-8.86 (m, 1H), 7.23-7.02 (m, 5H), 6.94-6.87 (m, 2H), 4.09-4.01 (m, 2H), 2.64-2.57 (m, 3H), 2.28-2.24 (m, 3H), 2.24-2.18 (m, 8H), 1.26-1.12 (m, 4H).

### Example 181: 5-(2-(Dimethylamino)ethoxy)-2-methyl-N-(1-(2-(trifluoromethyl)phenyl)cyclopropyl)benzamide (Compound 321)

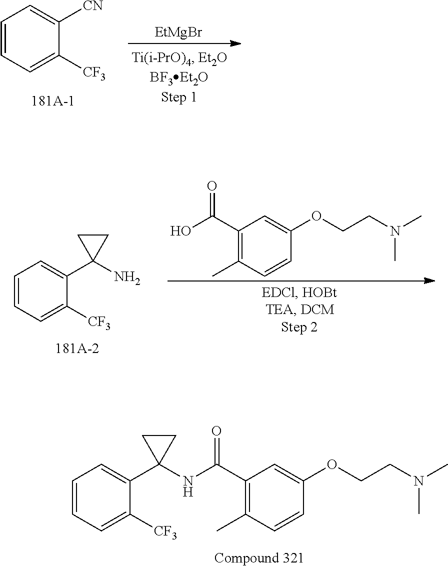 [SMILES: "EtMgBr" ; N#Cc1ccccc1C(F)(F)F ; NC1(c2ccccc2C(F)(F)F)CC1 ; Cc1ccc(OCCN(C)C)cc1C(=O)O ; Cc1ccc(OCCN(C)C)cc1C(=O)NC1(c2ccccc2C(F)(F)F)CC1]

### Step 1: 1-(2-(Trifluoromethyl)phenyl)cyclopropanamine (181A-2)

A mixture of 2-(trifluoromethyl)benzonitrile (200 mg, 1.17 mmol, 1.0 eq) in anhydrous Et₂O (15 mL) was degassed and purged with N₂ three times. The mixture was stirred with a mechanical stirrer at −78° C. To this mixture was added Ti(i-PrO)₄ (365 mg, 1.29 mmol, 379 μL, 1.1 eq) slowly, and then EtMgBr (3 M in Et₂O, 858 μL, 2.2 eq) was added dropwise to maintain the temperature between −78° C. and −75° C. over 1 h under a N₂ atmosphere. After the addition was complete, the resulting mixture was stirred at the same temperature for 10 min and warmed to 20° C. over 1 h. BF₃·Et₂O (331 mg, 2.34 mmol, 288 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (2.0 mL) and MTBE (15 mL), and extracted with MTBE (15 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (20 mL×2). The combined organic layers were dried over Na₂SO₄, filtered, and concentrated under vacuum to give a residue which was purified by preparative TLC (EtOAc/MeOH=10/1, R_{f}=0.2). 1-(2-(Trifluoromethyl)phenyl)cyclopropanamine (80.0 mg, 397 μmol, 34% yield) was obtained as a yellow oil. M+H⁺=202.0 (LCMS).

### Step 2: 5-(2-(Dimethylamino)ethoxy)-2-methyl-N-(1-(2-(trifluoromethyl)phenyl)cyclopropyl)benzamide (Compound 321)

To a solution of 1-(2-(trifluoromethyl)phenyl)cyclopropanamine (50.0 mg, 248 μmol, 1.0 eq) and 5-[2-(dimethylamino)ethoxy]-2-methyl-benzoic acid (55.5 mg, 248 μmol, 1.0 eq) in DMF (1.0 mL) were added HATU (142 mg, 372 μmol, 1.5 eq) and DIEA (95.4 mg, 745 μmol, 130 μL, 3.0 eq). The mixture was stirred at 25° C. for 1 h. LCMS indicated that the starting material was completely consumed, and the desired mass was detected. The reaction mixture was filtered and the filtrate was purified by preparative HPLC (Phenomenex Luna C18 column (80× 30 mm, 3 μm); flow rate: 25 mL/min; gradient: 5%-40% B over 8 min; mobile phase A: 0.04% aqueous HCl, mobile phase B: acetonitrile) to give 5-[2-(dimethylamino)ethoxy]-2-methyl-N-[1-[2-(trifluoromethyl)phenyl]cyclopropyl]benzamide (55.8 mg, 100 mmol, 51% yield, HCl salt) as a white solid. M+H⁺=407.1 (LCMS); ¹H NMR (400 MHZ, DMSO-d₆) δ 10.32 (br d, J=1.1 Hz, 1H), 8.68 (s, 1H), 8.01 (d, J=7.8 Hz, 1H), 7.69 (d, J=7.8 Hz, 1H), 7.62 (t, J=7.6 Hz, 1H), 7.52-7.45 (m, 1H), 7.12 (d, J=8.5 Hz, 1H), 6.93 (dd, J=2.6, 8.4 Hz, 1H), 6.74 (d, J=2.6 Hz, 1H), 4.29 (t, J=5.0 Hz, 2H), 3.47 (q, J=5.1 Hz, 2H), 2.81 (d, J=4.9 Hz, 6H), 2.08 (s, 3H), 1.30-1.14 (m, 4H).

### Example 182: 5-(2-(Dimethylamino)ethoxy)-N-(1-(2-ethylphenyl)cyclopropyl)-2-methylbenzamide (Compound 364)

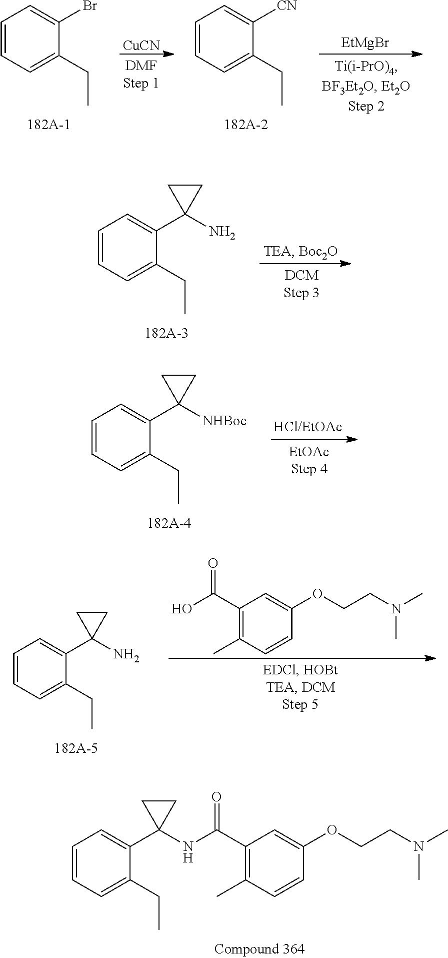

### Step 1: 2-Ethylbenzonitrile (182A-2)

To a solution of 1-bromo-2-ethylbenzene (2.00 g, 10.8 mmol, 1.49 mL, 1.0 eq) in DMF (17 mL) was added CuCN (1.45 g, 16.2 mmol, 3.54 mL, 1.5 eq). The mixture was stirred at 140° C. for 16 h. LCMS indicated that the starting material was completely consumed, and the desired mass was detected. The reaction mixture was allowed to cool to room temperature, poured into H₂O (15 mL) and extracted with EtOAc (15 mL×3). The combined organic layers were dried over Na₂SO₄, filtered, and concentrated under vacuum to give a residue which was purified by flash silica gel chromatography using a gradient of EtOAc/petroleum ether from 0/1 to 1/5. 2-Ethylbenzonitrile (800 mg, 6.10 mmol, 56% yield) was obtained as a white solid. M+H⁺=132.1 (LCMS).

### Step 2: 1-(2-Ethylphenyl)cyclopropanamine (182A-3)

A mixture of 2-ethylbenzonitrile (400 mg, 3.05 mmol, 411 μL, 1.0 eq) in anhydrous Et₂O (28 mL) was degassed and purged with N₂ three times. To this mixture was added Ti(i-PrO)₄ (953 mg, 3.35 mmol, 990 μL, 1.1 eq) slowly at −78° C., and then EtMgBr (3 M in Et₂O, 2.24 mL, 2.2 eq) was added dropwise to maintain the temperature between −78° C. and −75° C. over 5 min under a N₂ atmosphere. After the addition was complete, the resulting mixture was stirred at the same temperature for 10 min and warmed to 20° C. over 1 h. BF₃·Et₂O (866 mg, 6.10 mmol, 753 L, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (30 mL) and MTBE (30 mL), and extracted with MTBE (30 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (20 mL×4). The combined organic layers were dried over Na₂SO₄, filtered, and concentrated under vacuum to give the crude product 1-(2-ethylphenyl)cyclopropanamine (490 mg) as a white solid. M+H⁺=162.1 (LCMS).

### Step 3: tert-Butyl(1-(2-ethylphenyl)cyclopropyl)carbamate (182A-4)

To a solution of 1-(2-ethylphenyl)cyclopropanamine (490 mg, 3.04 mmol, 1.0 eq) in DCM (50 mL) were added TEA (615 mg, 6.08 mmol, 846 μL, 2.0 eq) and Boc₂O (663 mg, 3.04 mmol, 698 μL, 1.0 eq). The mixture was stirred at 20° C. for 15 h. LCMS indicated that the starting material was completely consumed, and the desired mass was detected. The reaction mixture was poured into H₂O (30 mL) and extracted with DCM (30 mL×3). The combined organic layers were dried over Na₂SO₄, filtered, and concentrated under vacuum to give a residue which was purified by flash silica gel chromatography using a gradient of EtOAc/petroleum ether from 0/1 to 1/5. tert-Butyl(1-(2-ethylphenyl)cyclopropyl)carbamate (70.0 mg, 268 μmol, 9% yield) was obtained as a white solid. M+H™=262.2 (LCMS).

### Step 4: 1-(2-Ethylphenyl)cyclopropanamine (182A-5)

To a stirred solution of tert-butyl(1-(2-ethylphenyl)cyclopropyl)carbamate (70.0 mg, 268 μmol, 1.0 eq) in EtOAc (1.5 mL) was added HCl/EtOAc (4 M, 3.0 mL). The mixture was stirred at 25° C. for 1 h. LCMS indicated that the starting material was completely consumed, and the desired mass was detected. The mixture was concentrated under vacuum at 30° C. to give the crude product 1-(2-ethylphenyl)cyclopropanamine (70 mg, HCl salt) as a white solid. M+H⁺=162.1 (LCMS).

### Step 5: 5-(2-(Dimethylamino)ethoxy)-N-(1-(2-ethylphenyl)cyclopropyl)-2-methylbenzamide (Compound 364)

To a solution of 1-(2-ethylphenyl)cyclopropanamine (60.0 mg, 372 μmol, 1.0 eq) and 5-(2-(dimethylamino)ethoxy)-2-methylbenzoic acid (83.1 mg, 372 μmol, 1.0 eq) in DCM (3.0 mL) were added TEA (113 mg, 1.12 mmol, 155 μL, 3.0 eq), EDCI (107 mg, 558 μmol, 1.5 eq) and HOBt (75.4 mg, 558 μmol, 1.5 eq). The mixture was stirred at 25° C. for 6 h. LCMS indicated that the starting material was completely consumed, and the desired mass was detected. The reaction mixture was poured into H₂O (5.0 mL) and extracted with DCM (5.0 mL×3). The combined organic layers were dried over Na₂SO₄, filtered, and concentrated under vacuum to give a residue which was purified by preparative HPLC (Phenomenex luna C18 column (75×30 mm, 3 μm); flow rate: 25 mL/min; gradient: 20%-55% B over 8 min; mobile phase A: 0.2% aqueous TFA, mobile phase B: acetonitrile). 5-(2-(Dimethylamino)ethoxy)-N-(1-(2-ethylphenyl)cyclopropyl)-2-methylbenzamide (4.5 mg, 10.7 μmol, 3% yield, FA salt) was obtained as a white solid. M+H⁺=367.1 (LCMS); ¹H NMR (400 MHZ, CDCl₃) δ 8.43-8.23 (m, 1H), 7.76 (d, J=7.6 Hz, 1H), 7.25 (s, 1H), 7.21 (br d, J=8.2 Hz, 1H), 7.09-7.02 (m, 1H), 6.84-6.77 (m, 2H), 6.56-6.54 (m, 1H), 6.50 (s, 1H), 4.25 (br t, J=4.8 Hz, 2H), 3.15 (br t, J=4.7 Hz, 2H), 3.03-2.90 (m, 2H), 2.66 (s, 6H), 2.21 (s, 3H), 1.41-1.34 (m, 2H), 1.31 (t, J=7.6 Hz, 3H), 1.28-1.23 (m, 2H).

### Example 183: 5-(2-(Dimethylamino)ethoxy)-N-(1-(2-isopropylphenyl)cyclopropyl)-2-methylbenzamide (Compound 344)

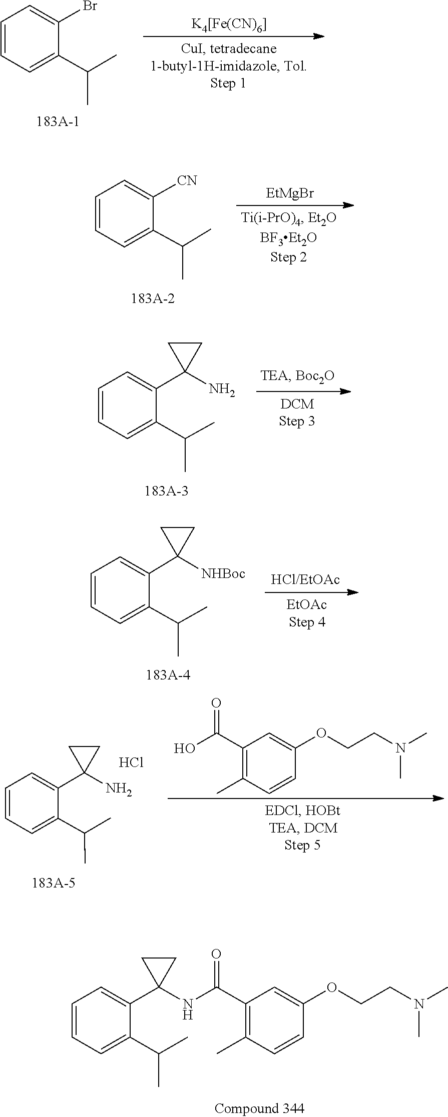

### Step 1: 2-Isopropylbenzonitrile (183A-2)

A mixture of K₄[Fe(CN)₆] (370 mg, 1.00 mmol, 0.2 eq), CuI (95.6 mg, 502 μmol, 0.1 eq) and 1-bromo-2-isopropyl-benzene (1.00 g, 5.02 mmol, 1.0 eq) in toluene (5.0 mL) was degassed and purged with N₂ three times. To the mixture were added tetradecane (381 mg, 1.92 mmol, 500 L, 0.38 eq) and 1-butyl-1H-imidazole (624 mg, 5.02 mmol, 5.00 mL, 1.0 eq). The mixture was stirred at 160° C. for 16 h under a N₂ atmosphere. TLC indicated that some of the starting material still remained and a new main spot was detected. The reaction mixture was poured into saturated aqueous NH₄Cl (2.0 mL) and extracted with EtOAc (2.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/20. 2-Isopropylbenzonitrile (330 mg, 2.27 mmol, 45% yield) was obtained as a white solid. M+H⁺=146.1 (LCMS).

### Step 2: 1-(2-Isopropylphenyl)cyclopropanamine (183A-3)

A mixture of 2-isopropylbenzonitrile (330 mg, 2.27 mmol, 1.0 eq) in anhydrous Et₂O (23 mL) was degassed and purged with N₂ three times. To this mixture was added Ti(i-PrO)₄ (710 mg, 2.50 mmol, 738 μL, 1.1 eq) slowly at −78° C., and then EtMgBr (3 M in Et₂O, 1.67 mL, 2.2 eq) was added dropwise to maintain the temperature between −78° C. and −75° C. over 5 min under a N₂ atmosphere. After the addition was complete, the resulting mixture was stirred at the same temperature for 10 min and warmed to 20° C. over 1 h. BF₃·Et₂O (645 mg, 4.55 mmol, 561 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (30 mL) and MTBE (30 mL), and extracted with MTBE (30 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (20 mL×4). The combined organic layers were dried over Na₂SO₄, filtered, and concentrated under vacuum to give the crude product 1-(2-isopropylphenyl)cyclopropanamine (390 mg) as a white solid. M+H⁺=176.2 (LCMS).

### Step 3: tert-Butyl(1-(2-isopropylphenyl)cyclopropyl)carbamate (183A-4)

To a solution of 1-(2-isopropylphenyl)cyclopropanamine (390 mg, 2.23 mmol, 1.0 eq) in DCM (50 mL) were added TEA (450 mg, 4.45 mmol, 619 μL, 2.0 eq) and Boc₂O (583 mg, 2.67 mmol, 613 μL, 1.2 eq). The mixture was stirred at 20° C. for 15 h. LCMS indicated that the starting material was completely consumed, and the desired mass was detected. The reaction mixture was poured into H₂O (20 mL) and extracted with DCM (20 mL×3). The combined organic layers were dried over Na₂SO₄, filtered, and concentrated under vacuum to give a residue which was purified by flash silica gel chromatography using a gradient of EtOAc/petroleum ether from 0/1 to 1/5. tert-Butyl(1-(2-isopropylphenyl)cyclopropyl)carbamate (100 mg, 363 μmol, 16% yield) was obtained as a white solid. M−56+H⁺=220.1 (LCMS).

### Step 4: 1-(2-Isopropylphenyl)cyclopropanamine (183A-5)

To a stirred solution of tert-butyl(1-(2-isopropylphenyl)cyclopropyl)carbamate (100 mg, 363 μmol, 1.0 eq) in EtOAc (2.0 mL) was added HCl/EtOAc (4 M, 4.1 mL). The mixture was stirred at 25° C. for 1 h. LCMS indicated that the starting material was completely consumed, and the desired mass was detected. The mixture was concentrated under vacuum to give the crude product 1-(2-isopropylphenyl)cyclopropanamine (100 mg, HCl salt) as a white solid. M+H⁺=176.2 (LCMS).

### Step 5: 5-(2-(Dimethylamino)ethoxy)-N-(1-(2-isopropylphenyl)cyclopropyl)-2-methyl benzamide (Compound 344)

To a solution of 1-(2-isopropylphenyl)cyclopropanamine (90.0 mg, 513 μmol, 1.0 eq) and 5-(2-(dimethylamino)ethoxy)-2-methylbenzoic acid (91.7 mg, 411 μmol, 0.8 eq) in DCM (3.0 mL) were added TEA (156 mg, 1.54 mmol, 214 μL, 3.0 eq), EDCI (148 mg, 770 μmol, 1.5 eq) and HOBt (104 mg, 770 μmol, 1.5 eq). The mixture was stirred at 25° C. for 3 h. LCMS indicated that the starting material was completely consumed, and the desired mass was detected. The reaction mixture was poured into H₂O (5.0 mL) and extracted with DCM (5.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 15%-45% B over 8 min; mobile phase A: 0.2% aqueous FA, mobile phase B: acetonitrile). 5-(2-(Dimethylamino)ethoxy)-N-(1-(2-isopropylphenyl)cyclopropyl)-2-methylbenzamide (15.6 mg, 36.6 μmol, 7% yield, FA salt) was obtained as a white solid. M+H⁺=381.2 (LCMS); ¹H NMR (400 MHZ, CDCl₃) δ 8.46 (br s, 1H), 7.74 (d, J=7.7 Hz, 1H), 7.29 (d, J=4.0 Hz, 2H), 7.18 (dd, J=4.0, 7.6 Hz, 1H), 7.05 (d, J=8.3 Hz, 1H), 6.89-6.69 (m, 2H), 6.32 (s, 1H), 4.04 (t, J=5.5 Hz, 2H), 3.82-3.49 (m, 1H), 2.78 (t, J=5.4 Hz, 2H), 2.38 (s, 6H), 2.22 (s, 3H), 1.44-1.33 (m, 2H), 1.28 (d, J=7.0 Hz, 6H), 1.25 (br s, 2H).

### Example 184: N-(1-([1,1′-Biphenyl]-2-yl)cyclopropyl)-5-(2-(dimethylamino)ethoxy)-2-methylbenzamide (Compound 267)

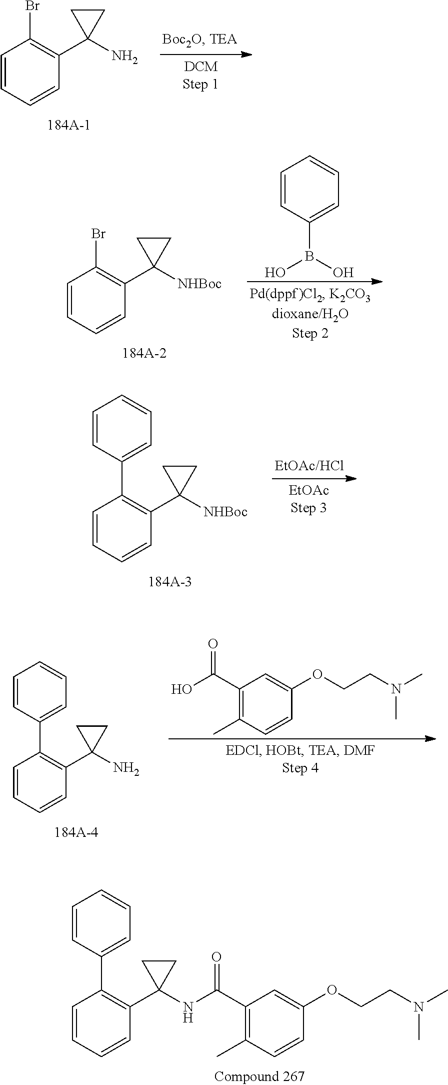

### Step 1: tert-Butyl(1-(2-bromophenyl)cyclopropyl)carbamate (184A-2)

To a solution of 1-(2-bromophenyl)cyclopropanamine (500 mg, 2.36 mmol, 1.0 eq) in DCM (13 mL) were added di-tert-butyl dicarbonate (515 mg, 2.36 mmol, 542 μL, 1.0 eq) and TEA (262 mg, 2.59 mmol, 361 μL, 1.1 eq). The mixture was stirred at 25° C. for 16 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to tert-Butyl(1-(2-bromophenyl)cyclopropyl)carbamate (660 mg, 2.11 mmol, 90% yield) was obtained as a yellow gum. ¹H NMR (400 MHZ, CDCl₃) δ 7.56-7.51 (m, 1H), 7.30-7.27 (m, 1H), 7.24 (s, 1H), 7.16-7.10 (m, 1H), 1.44-1.33 (m, 9H), 1.27-1.21 (m, 2H), 1.17-1.12 (m, 2H).

### Step 2: tert-Butyl(1-([1,1′-biphenyl]-2-yl)cyclopropyl)carbamate (184A-3)

A mixture of phenylboronic acid (195 mg, 1.60 mmol, 2.5 eq), tert-butyl(1-(2-bromophenyl)cyclopropyl)carbamate (200 mg, 641 μmol, 1.0 eq), K₂CO₃ (221 mg, 1.60 mmol, 2.5 eq), Pd(dppf)Cl₂ (46.9 mg, 64.1 μmol, 0.1 eq) in a mixture of dioxane (8.0 mL) and H₂O (2.0 mL) was stirred at 80° C. for 16 h under a N₂ atmosphere. LCMS indicated that the starting material was completely consumed, and the desired mass was detected. The reaction mixture was allowed to cool to room temperature, poured into H₂O (10 mL) and extracted with DCM (10 mL×5). The combined organic layers were dried over Na₂SO₄, filtered, and concentrated under vacuum to give a residue which was purified by flash silica gel chromatography using a gradient of EtOAc/petroleum ether from 0/1 to 1/3. tert-Butyl(1-([1,1′-biphenyl]-2-yl)cyclopropyl)carbamate (150 mg, 485 μmol, 76% yield) was obtained as a white solid.

### Step 3: 1-([1,1′-Biphenyl]-2-yl)cyclopropanamine (184A-4)

To a solution of tert-butyl(1-([1, l′-biphenyl]-2-yl)cyclopropyl)carbamate (100 mg, 323 μmol, 1.0 eq) in EtOAc (1.0 mL) was added HCl/EtOAc (4 M, 3.0 mL). The resulting mixture was stirred at 25° C. for 30 min. LCMS indicated that the starting material was completely consumed, and the desired mass was detected. The reaction mixture was concentrated under vacuum to give the crude product 1-([1, l′-biphenyl]-2-yl)cyclopropanamine (75.0 mg, HCl salt) as a white solid. M+H⁺=210.1 (LCMS).

### Step 4: N-(1-([1,1′-Biphenyl]-2-yl)cyclopropyl)-5-(2-(dimethylamino)ethoxy)-2-methyl benzamide (Compound 267)

To a solution of 5-(2-(dimethylamino)ethoxy)-2-methylbenzoic acid (68.1 mg, 305 μmol, 1.0 eq) and 1-([1,1′-biphenyl]-2-yl)cyclopropanamine (75.0 mg, 305 μmol, 1.0 eq, HCl salt) in DMF (5.0 mL) were added TEA (92.7 mg, 916 μmol, 127 μL, 3.0 eq), EDCI (70.2 mg, 366 μmol, 1.2 eq) and HOBt (49.5 mg, 366 μmol, 1.2 eq). The mixture was stirred at 25° C. for 4 h. LCMS indicated that the starting material was completely consumed, and the desired mass was detected. The reaction mixture was poured into H₂O (10 mL) and extracted with DCM (5.0 mL×5). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 1%-40% B over 8 min; mobile phase A: 0.2% aqueous FA, mobile phase B: acetonitrile). N-(1-([1,1′-Biphenyl]-2-yl)cyclopropyl)-5-(2-(dimethylamino)ethoxy)-2-methylbenzamide (31.6 mg, 68.4 μmol, 22% yield, FA salt) was obtained as a yellow solid. M+H⁺=415.1 (LCMS); ¹H NMR (400 MHZ, DMSO-d₆) δ 8.20 (br s, 1H), 8.05 (s, 1H), 7.94-7.87 (m, 1H), 7.51-7.47 (m, 2H), 7.43 (t, J=7.5 Hz, 2H), 7.38-7.27 (m, 3H), 7.14 (dd, J=1.9, 7.0 Hz, 1H), 7.04 (d, J=8.5 Hz, 1H), 6.82 (dd, J=2.6, 8.4 Hz, 1H), 6.48 (d, J=2.6 Hz, 1H), 3.98 (t, J=5.7 Hz, 2H), 2.64 (br t, J=5.6 Hz, 2H), 2.24 (s, 6H), 2.09 (s, 3H), 1.03-0.91 (m, 4H).

### Example 185: 5-(2-(Dimethylamino)ethoxy)-N-(1-(4-hydroxyphenyl)cyclopropyl)-2-methylbenzamide (Compound 294)

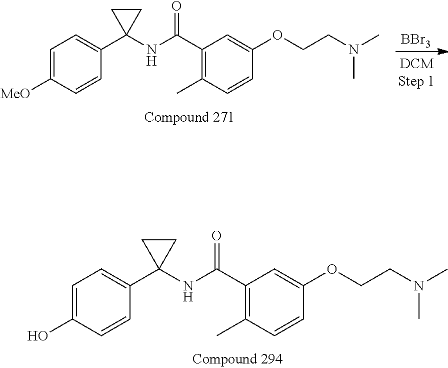

### Step 1: 5-(2-(Dimethylamino)ethoxy)-N-(1-(4-hydroxyphenyl)cyclopropyl)-2-methylbenzamide (Compound 294)

A mixture of 5-(2-(dimethylamino)ethoxy)-N-(1-(4-methoxyphenyl)cyclopropyl)-2-methylbenzamide (100 mg, 265 μmol, 1.0 eq) in DCM (1.0 mL) was degassed and purged with N₂ three times. The mixture was added a solution of BBr₃ (1.22 g, 4.89 mmol, 471 μL, 20 eq) in DCM (1.0 mL) at −78° C. and stirred at the same temperature for 1 h. LCMS indicated that the starting material was completely consumed, and the desired mass was detected. The mixture was concentrated under vacuum at 20° C. to give a residue which was purified by preparative HPLC (Phenomenex Luna C18 column (80×30 mm, 3 μm); flow rate: 25 mL/min; gradient: 10%-40% B over 8 min; mobile phase A: 0.04% aqueous HCl, mobile phase B: acetonitrile). 5-(2-(Dimethylamino)ethoxy)-N-(1-(4-hydroxyphenyl)cyclopropyl)-2-methylbenzamide (5.50 mg, 15.4 μmol, 6% yield, HCl salt) was obtained as a yellow oil. M+H⁺=355.1 (LCMS); ¹H NMR (400 MHZ, DMSO-d₆) δ 7.15 (d, J=8.6 Hz, 1H), 7.10-7.05 (m, 2H), 6.94 (dd, J=2.8, 8.4 Hz, 1H), 6.87 (d, J=2.7 Hz, 1H), 6.70-6.62 (m, 2H), 4.28-4.19 (m, 2H), 3.48-3.43 (m, 2H), 2.81 (s, 6H), 2.17 (s, 3H), 1.15-1.07 (m, 4H).

### Example 186: 5-(2-(Dimethylamino)ethoxy)-N-(1-(4-methoxyphenyl)cyclopropyl)-2-methylbenzamide (Compound 271)

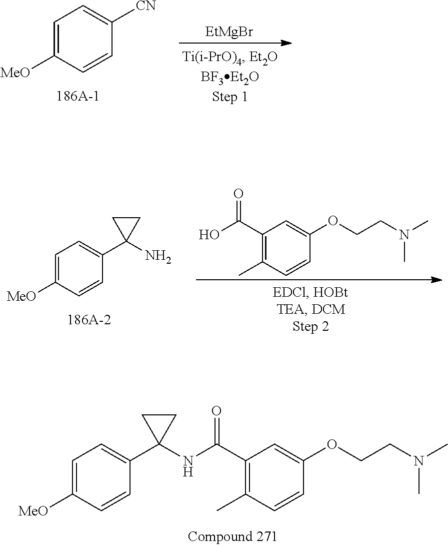 [SMILES: "EtMgBr" ; COc1ccc(C#N)cc1 ; COc1ccc(C2(N)CC2)cc1 ; Cc1ccc(OCCN(C)C)cc1C(=O)O ; COc1ccc(C2(NC(=O)c3cc(OCCN(C)C)ccc3C)CC2)cc1]

### Step 1: 1-(4-Methoxyphenyl)cyclopropanamine (186A-2)

A mixture of 4-methoxybenzonitrile (2.00 g, 15.0 mmol, 1.0 eq) in anhydrous Et₂O (100 mL) was degassed and purged with N₂ three times. To this mixture was added Ti(i-PrO)₄ (4.27 g, 15.0 mmol, 4.43 mL, 1.0 eq) slowly at −78° C., and then EtMgBr (3 M in Et₂O, 11.0 mL, 2.2 eq) was added dropwise to maintain the temperature between −78° C. and −75° C. over 30 min under a N₂ atmosphere. After the addition was complete, the resulting mixture was stirred at the same temperature for 10 min and warmed to 20° C. over 1 h. BF₃·Et₂O (4.26 g, 30.0 mmol, 3.71 mL, 2.0 eq) was added slowly with no obvious temperature change. The mixture was stirred at 20° C. for another 1 h. TLC indicated that the starting material was completely consumed. The reaction mixture was added into a mixture of HCl (1 M aqueous) (30 mL) and MTBE (30 mL) and extracted with MTBE (30 mL×3). The aqueous layer was basified to pH 8 using NaOH (2 M aqueous) and a precipitate was formed. The mixture was filtered through a pad of Celite and the slurry was washed with DCM several times. The combined filtrate was extracted with DCM (50 mL×2). The combined organic layers were dried over Na₂SO₄, filtered, and concentrated under vacuum to give a residue which was purified by flash silica gel chromatography using a gradient of EtOAc/petroleum ether from 0/1 to 4/1. 1-(4-Methoxyphenyl)cyclopropanamine (1.30 g, 7.96 mmol, 53% yield) was obtained as a yellow oil. M+H⁺=164.1 (LCMS).

### Step 2: 5-(2-(Dimethylamino)ethoxy)-N-(1-(4-methoxyphenyl)cyclopropyl)-2-methyl benzamide (Compound 271)

To a solution of 1-(4-methoxyphenyl)cyclopropanamine (100 mg, 447 μmol, 1.2 eq), 5-(2-(dimethylamino)ethoxy)-2-methylbenzoic acid (83.2 mg, 373 μmol, 1.0 eq) in DCM (2.0 mL) were added TEA (113 mg, 1.12 mmol, 156 μL, 3.0 eq), EDCI (107 mg, 559 mmol, 1.5 eq) and HOBt (75.5 mg, 559 mmol, 1.5 eq). The mixture was stirred at 25° C. for 16 h. LCMS indicated that the starting material was completely consumed, and the desired mass was detected. The reaction mixture was poured into H₂O (10 mL) and extracted with EtOAc (10 mL×2). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 10%-40% B over 8 min; mobile phase A: 0.04% aqueous HCl, mobile phase B: acetonitrile). 5-(2-(Dimethylamino)ethoxy)-N-(1-(4-methoxyphenyl)cyclopropyl)-2-methylbenzamide (100 mg, 265 μmol, 71% yield, HCl salt) was obtained as a white solid. M+H⁺=369.1 (LCMS); ¹H NMR (400 MHZ, CDCl₃) δ 12.80 (br s, 1H), 7.43-7.36 (m, 2H), 7.07 (d, J=8.4 Hz, 1H), 6.96 (d, J=2.6 Hz, 1H), 6.90-6.77 (m, 4H), 4.54-4.47 (m, 2H), 3.80 (s, 3H), 3.46-3.39 (m, 2H), 2.93 (d, J=4.9 Hz, 6H), 2.31 (s, 3H), 1.38-1.32 (m, 2H), 1.31-1.26 (m, 2H).

### Example 187: 5-(2-(Dimethylamino)ethoxy)-2-methyl-N-(1-(2-phenoxyphenyl)cyclopropyl)benzamide (Compound 298)

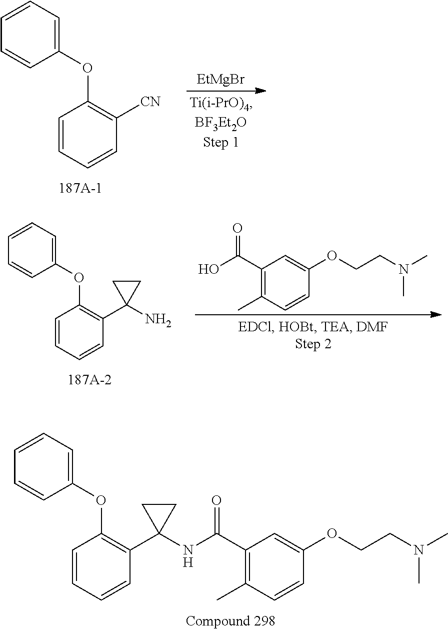 [SMILES: "EtMgBr" ; N#Cc1ccccc1Oc1ccccc1 ; NC1(c2ccccc2Oc2ccccc2)CC1 ; Cc1ccc(OCCN(C)C)cc1C(=O)O ; Cc1ccc(OCCN(C)C)cc1C(=O)NC1(c2ccccc2Oc2ccccc2)CC1]

### Step 1: 1-(2-Phenoxyphenyl)cyclopropanamine (187A-2)

A mixture of 2-phenoxybenzonitrile (300 mg, 1.54 mmol, 1.0 eq) in anhydrous Et₂O (20 mL) was degassed and purged with N₂ three times. To this mixture was added Ti(i-PrO)₄ (480 mg, 1.69 mmol, 499 μL, 1.1 eq) slowly at −78° C., and then EtMgBr (3 M in Et₂O, 1.13 mL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. BF₃·Et₂O (436 mg, 3.07 mmol, 379 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (15 mL) and MTBE (15 mL), and extracted with MTBE (10 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (10 mL×2). The combined organic layers were dried over Na₂SO₄, filtered, and concentrated under vacuum to give a residue which was purified by flash silica gel chromatography using a gradient of EtOAc/petroleum ether from 0/1 to 1/0. 1-(2-Phenoxyphenyl)cyclopropanamine (90.0 mg, 399 μmol, 26% yield) was obtained as a yellow oil. M+H⁺=226.1 (LCMS); ¹H NMR (400 MHZ, CDCl₃) δ 7.41-7.33 (m, 3H), 7.21 (dt, J=1.6, 7.8 Hz, 1H), 7.15-6.98 (m, 4H), 6.87 (dd, J=0.9, 8.1 Hz, 1H), 1.00 (s, 2H), 0.91-0.86 (m, 2H).

### Step 2: 5-(2-(Dimethylamino)ethoxy)-2-methyl-N-(1-(2-phenoxyphenyl)cyclopropyl)benzamide (Compound 298)

To a solution of 5-(2-(dimethylamino)ethoxy)-2-methylbenzoic acid (178 mg, 799 μmol, 2.0 eq) and 1-(2-phenoxyphenyl)cyclopropanamine (90.0 mg, 399 μmol, 1.0 eq) in DMF (5.0 mL) were added TEA (121 mg, 1.20 mmol, 167 μL, 3.0 eq), EDCI (91.9 mg, 479 μmol, 1.2 eq) and HOBt (64.8 mg, 479 μmol, 1.2 eq). The mixture was stirred at 25° C. for 16 h. LCMS indicated that the starting material was completely consumed, and the desired mass was detected. The reaction mixture was poured into H₂O (10 mL) and extracted with DCM (5.0 mL×5). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 10%-50% B over 8 min; mobile phase A: 0.2% aqueous FA, mobile phase B: acetonitrile). 5-(2-(Dimethylamino)ethoxy)-2-methyl-N-(1-(2-phenoxyphenyl)cyclopropyl)benzamide (51.3 mg, 108 μmol, 27% yield, FA) was obtained as a yellow gum. M+H⁺=431.1 (LCMS); ¹H NMR (400 MHZ, DMSO-d₆) δ 8.74 (s, 1H), 8.17 (s, 1H), 7.64 (br d, J=7.6 Hz, 1H), 7.37 (br t, J=7.9 Hz, 2H), 7.25-7.20 (m, 1H), 7.13-6.99 (m, 5H), 6.82 (br dd, J=2.1, 8.3 Hz, 1H), 6.75 (br d, J=8.0 Hz, 1H), 6.67 (d, J=2.0 Hz, 1H), 3.93 (br t, J=5.5 Hz, 2H), 2.60 (br s, 2H), 2.22 (s, 6H), 2.14 (s, 3H), 1.11 (br d, J=9.9 Hz, 4H).

### Example 188: 5-(2-(Dimethylamino)ethoxy)-2-methyl-N-(1-(3-phenoxyphenyl)cyclopropyl)benzamide (Compound 270)

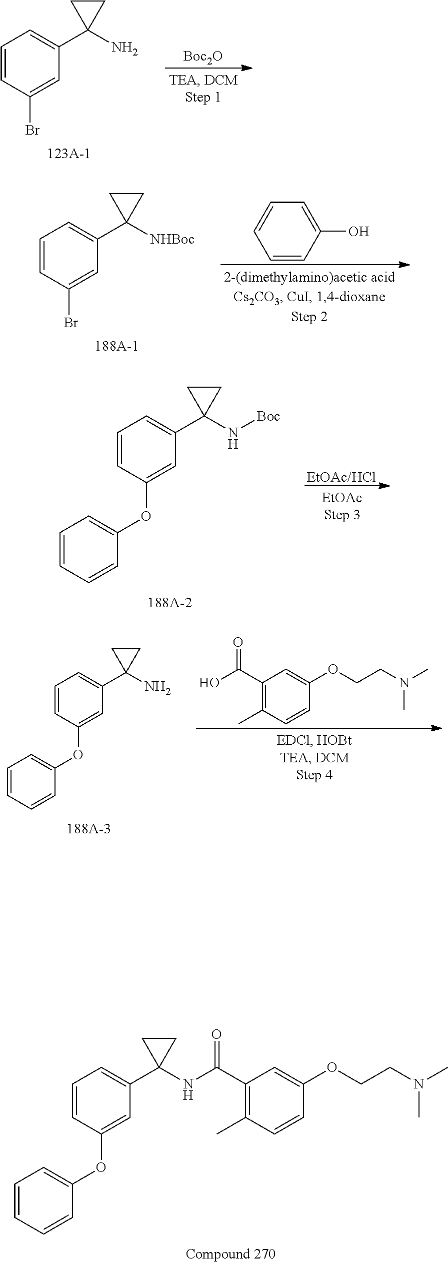

### Step 1: tert-Butyl(1-(3-bromophenyl)cyclopropyl)carbamate (188A-1)

To a stirred solution of 1-(3-bromophenyl)cyclopropanamine (250 mg, 1.18 mmol, 1.0 eq) in DCM (5.0 mL) were added TEA (131 mg, 1.30 mmol, 180 μL, 1.1 eq) and Boc₂O (257 mg, 1.18 mmol, 271 μL, 1.0 eq). The mixture was stirred at 20° C. for 16 h. LCMS indicated that the starting material was completely consumed, and the desired mass was detected. The mixture was concentrated under vacuum at 30° C. to give a residue which was purified by preparative TLC (EtOAc/petroleum ether=1/3, R_{f}=0.5). tert-Butyl(1-(3-bromophenyl)cyclopropyl) carbamate (300 mg, 961 μmol, 82% yield) was obtained as a white solid. M−56+H⁺=255.9 (LCMS).

### Step 2: tert-Butyl(1-(3-phenoxyphenyl)cyclopropyl)carbamate (188A-2)

A mixture of tert-butyl(2-hydroxyethyl)carbamate (50.0 mg, 160 μmol, 1.0 eq), phenol (21.1 mg, 224 μmol, 1.4 eq), Cs₂CO₃ (104 mg, 320 μmol, 2.0 eq) and CuI (3.05 mg, 16.0 μmol, 0.1 eq) in 1,4-dioxane (3.0 mL) was degassed and purged with N₂ three times. To the mixture was added 2-(dimethylamino) acetic acid (4.95 mg, 48.1 μmol, 0.3 eq). The resulting mixture was stirred at 110° C. for 16 h under a N₂ atmosphere. LCMS indicated that the starting material was completely consumed, and the desired mass was detected. The reaction mixture was allowed to cool to room temperature, poured into H₂O (3.0 mL) and extracted with EtOAc (3.0 mL×3). The combined organic layers were dried over Na₂SO₄, filtered, and concentrated under vacuum to give a residue which was purified by preparative TLC (EtOAc/petroleum ether=1/3, R_{f}=0.6). tert-Butyl(1-(3-phenoxyphenyl)cyclopropyl)carbamate (45.0 mg, 138 μmol, 86% yield) was obtained as a white solid. M+H⁺=326.1 (LCMS).

### Step 3: 1-(3-Phenoxyphenyl)cyclopropanamine (188A-3)

To a stirred solution of tert-butyl(1-(3-phenoxyphenyl)cyclopropyl)carbamate (90.0 mg, 277 μmol, 1.0 eq) in EtOAc (5.0 mL) was added HCl/EtOAc (4 M, 3.0 mL). The mixture was stirred at 20° C. for 1 h. LCMS indicated that the starting material was completely consumed, and the desired mass was detected. The mixture was concentrated under vacuum at 30° C. to give the crude product 1-(3-phenoxyphenyl)cyclopropanamine (50.0 mg, 168 μmol, 60% yield, HCl salt) as a white solid. M+H⁺=226.0 (LCMS).

### Step 4: 5-(2-(Dimethylamino)ethoxy)-2-methyl-N-(1-(3-phenoxyphenyl)cyclopropyl)benzamide (Compound 270)

To a solution of 1-(3-phenoxyphenyl)cyclopropanamine (100 mg, 382 μmol, 1.0 eq, HCl salt) and 5-(2-(dimethylamino)ethoxy)-2-methylbenzoic acid (85.3 mg, 382 μmol, 1.0 eq) in DCM (3.0 mL) were added TEA (77.3 mg, 764 μmol, 106 μL, 2.0 eq), EDCI (110 mg, 573 μmol, 1.5 eq) and HOBt (77.4 mg, 573 μmol, 1.5 eq). The mixture was stirred at 20° C. for 2 h. LCMS indicated that the starting material was completely consumed, and the desired mass was detected. The reaction mixture was poured into H₂O (3.0 mL) and extracted with DCM (3.0 mL×2). The combined organic layers were dried over Na₂SO₄, filtered, and concentrated under vacuum to give a residue which was purified by preparative HPLC (Phenomenex Gemini C18 column (75×30 mm, 3 μm); flow rate: 25 mL/min; gradient: 45%-80% B over 8 min; mobile phase A: 10 mM aqueous NH₄HCO₃, mobile phase B: acetonitrile). 5-(2-(Dimethylamino)ethoxy)-2-methyl-N-(1-(3-phenoxyphenyl)cyclopropyl)benzamide (24.4 mg, 52.1 μmol, 14% yield) was obtained as a yellow gum. M+H⁺=431.1 (LCMS); ¹H NMR (400 MHz, DMSO-d₆) δ 8.90 (s, 1H), 7.43-7.26 (m, 3H), 7.17-7.07 (m, 2H), 7.04-6.96 (m, 2H), 6.95-6.86 (m, 3H), 6.84-6.77 (m, 2H), 4.01 (t, J=5.8 Hz, 2H), 2.61 (t, J=5.8 Hz, 2H), 2.21 (s, 6H), 2.15 (s, 3H), 1.25 (br d, J=1.4 Hz, 4H).

### Example 189: 5-(2-(Dimethylamino)ethoxy)-2-methyl-N-(1-(3-phenoxyphenyl)cyclopropyl)benzamide (Compound 279)

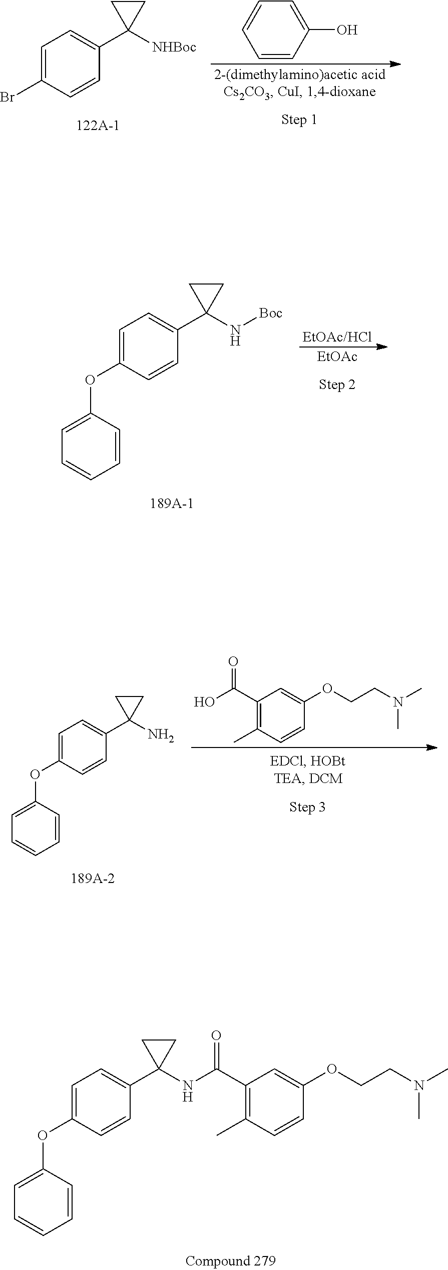

### Step 1: tert-butyl(1-(4-phenoxyphenyl)cyclopropyl)carbamate (189A-1)

A mixture of tert-butyl(1-(4-bromophenyl)cyclopropyl)carbamate (100 mg, 320 μmol, 1.0 eq), phenol (42.2 mg, 448 μmol, 1.4 eq), Cs₂CO₃ (209 mg, 640 μmol, 2.0 eq) and CuI (6.10 mg, 32.0 μmol, 0.1 eq) in 1,4-dioxane (5.0 mL) was degassed and purged with N₂ three times. To the mixture was added 2-(dimethylamino) acetic acid (9.91 mg, 96.1 μmol, 0.3 eq) and the resulting mixture was stirred at 110° C. for 16 h under a N₂ atmosphere. LCMS indicated that the starting material was completely consumed, and the desired mass was detected. The reaction mixture was allowed to cool to room temperature, poured into H₂O (5.0 mL) and extracted with EtOAc (5.0 mL×3). The combined organic layers were dried over Na₂SO₄, filtered, and concentrated under vacuum to give a residue which was purified by preparative TLC (EtOAc/petroleum ether=1/3, R_{f}=0.7). tert-Butyl(1-(4-phenoxy phenyl)cyclopropyl) carbamate (100 mg, 307 μmol, 96% yield) was obtained as a yellow oil. M+H⁺=326.1 (LCMS).

### Step 2: 1-(4-Phenoxyphenyl)cyclopropanamine (189A-2)

To a stirred solution of tert-butyl(1-(4-phenoxyphenyl)cyclopropyl)carbamate (100 mg, 307 μmol, 1.0 eq) in EtOAc (5.0 mL) was added HCl/EtOAc (4 M, 3.0 mL). The mixture was stirred at 20° C. for 2 h. LCMS indicated that the starting material was completely consumed, and the desired mass was detected. The mixture was concentrated under vacuum at 30° C. to give the crude product 1-(4-phenoxyphenyl)cyclopropanamine (100 mg, HCl salt) as a white solid. M+H⁺=226.0 (LCMS).

### Step 3: 5-(2-(Dimethylamino)ethoxy)-2-methyl-N-(1-(4-phenoxyphenyl)cyclopropyl)benzamide (Compound 279)

To a solution of 1-(4-phenoxyphenyl)cyclopropanamine (100 mg, 382 μmol, 1.0 eq, HCl salt) and 5-(2-(dimethylamino)ethoxy)-2-methylbenzoic acid (85.3 mg, 382 μmol, 1.0 eq) in DCM (3.0 mL) were added TEA (77.3 mg, 764 μmol, 106 μL, 2.0 eq), EDCI (110 mg, 573 μmol, 1.5 eq) and HOBt (77.4 mg, 573 μmol, 1.5 eq). The mixture was stirred at 20° C. for 2 h. LCMS indicated that the starting material was completely consumed, and the desired mass was detected. The reaction mixture was poured into H₂O (3.0 mL) and extracted with DCM (3.0 mL×2). The combined organic layers were dried over Na₂SO₄, filtered, and concentrated under vacuum to give a residue which was purified by preparative HPLC (Phenomenex Gemini C18 column (100×30 mm, 10 μm); flow rate: 25 mL/min; gradient: 50%-70% B over 8 min; mobile phase A: 10 mM aqueous NH₄HCO₃, mobile phase B: acetonitrile). 5-(2-(Dimethylamino)ethoxy)-2-methyl-N-(1-(4-phenoxyphenyl)cyclopropyl)benzamide (15.0 mg, 34.8 μmol, 9% yield) was obtained as a white solid. M+H⁺=431.1 (LCMS); ¹H NMR (400 MHz, DMSO-d₆) δ 8.93 (s, 1H), 7.42-7.33 (m, 2H), 7.25 (d, J=8.8 Hz, 2H), 7.12 (d, J=7.5 Hz, 2H), 7.02-6.87 (m, 6H), 4.04 (t, J=5.8 Hz, 2H), 2.60 (br t, J=5.8 Hz, 2H), 2.27-2.15 (m, 9H), 1.23 (br d, J=7.3 Hz, 4H).

### Example 190: N-(1-(2-(Benzyloxy)phenyl)cyclopropyl)-5-(2-(dimethylamino)ethoxy)-2-methylbenzamide (Compound 327)

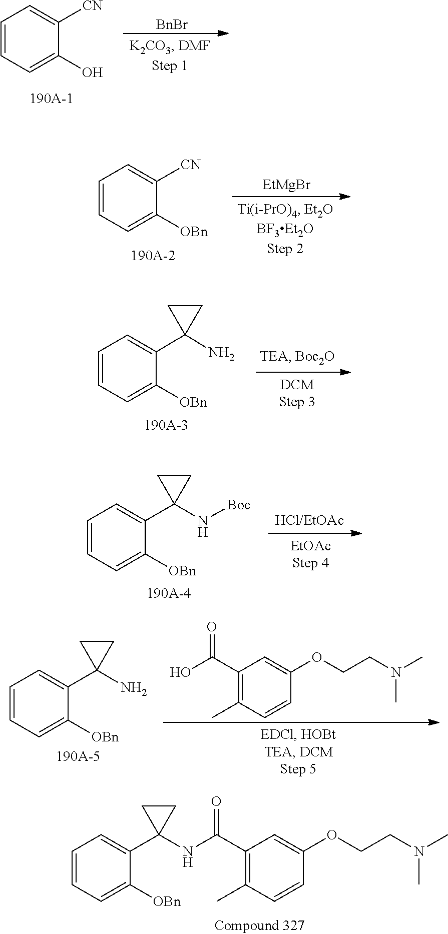

### Step 1: 2-(Benzyloxy)benzonitrile (190A-2)

To a solution of 2-hydroxybenzonitrile (2.00 g, 16.8 mmol, 1.0 eq) and K₂CO₃ (4.64 g, 33.6 mmol, 2.0 eq) in DMF (20 mL) was added (bromomethyl)benzene (3.45 g, 20.2 mmol, 2.39 mL, 1.2 eq). The mixture was stirred at 50° C. for 3 h. LCMS indicated that the starting material was completely consumed, and the desired mass was detected. The reaction mixture was allowed to cool to room temperature, poured into H₂O (20 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/0 to 1/10. 2-(Benzyloxy)benzonitrile (3.20 g, 15.3 mmol, 91% yield) was obtained as a white solid. M+H⁺=210.1 (LCMS); ¹H NMR (400 MHZ, CDCl₃) δ 7.59 (dd, J=1.6, 7.9 Hz, 1H), 7.55-7.44 (m, 3H), 7.44-7.38 (m, 2H), 7.38-7.33 (m, 1H), 7.08-6.96 (m, 2H), 5.23 (s, 2H).

### Step 2: 1-(2-(Benzyloxy)phenyl)cyclopropanamine (190A-3)

A mixture of 2-(benzyloxy)benzonitrile (500 mg, 2.39 mmol, 1.0 eq) in anhydrous Et₂O (50 mL) was degassed and purged with N₂ three times. The mixture was stirred at −78° C. To this mixture was added Ti(i-PrO)₄ (747 mg, 2.63 mmol, 776 μL, 1.1 eq) slowly, and then EtMgBr (3 M in Et₂O, 1.75 mL, 2.2 eq) was added dropwise to maintain the temperature between-78° C. and −75° C. over 5 min under a N₂ atmosphere. After the addition was complete, the resulting mixture was stirred at the same temperature for 10 min and warmed to 20° C. over 1 h. BF₃·Et₂O (679 mg, 4.78 mmol, 590 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (30 mL) and MTBE (30 mL), and extracted with MTBE (30 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (20 mL×4). The combined organic layers were dried over Na₂SO₄, filtered, and concentrated under vacuum to give crude product 1-(2-(benzyloxy)phenyl)cyclopropanamine (580 mg) as a brown oil. M+H⁺=240.1 (LCMS).

### Step 3: tert-Butyl(1-(2-(benzyloxy)phenyl)cyclopropyl)carbamate (190A-4)

To a solution of 1-(2-(benzyloxy)phenyl)cyclopropanamine (570 mg, 2.38 mmol, 1.0 eq) in DCM (30 mL) were added TEA (482 mg, 4.76 mmol, 663 μL, 2.0 eq) and Boc₂O (624 mg, 2.86 mmol, 657 μL, 1.2 eq). The mixture was stirred at 20° C. for 16 h. LCMS indicated that the starting material was completely consumed, and the desired mass was detected. The reaction mixture was poured into H₂O (30 mL) and extracted with DCM (20 mL×3). The combined organic layers were dried over Na₂SO₄, filtered, and concentrated under vacuum to give a residue which was purified by flash silica gel chromatography using a gradient of EtOAc/petroleum ether from 1/0 to 1/7. tert-Butyl(1-(2-(benzyloxy)phenyl)cyclopropyl)carbamate (110 mg, 324 μmol, 14% yield) was obtained as a white solid.

### Step 4: 1-(2-(Benzyloxy)phenyl)cyclopropanamine (190A-5)

To a stirred solution of tert-butyl(1-(2-(benzyloxy)phenyl)cyclopropyl)carbamate (100 mg, 295 μmol, 1.0 eq) in EtOAc (2.0 mL) was added HCl/EtOAc (4 M, 5.2 mL). The mixture was stirred at 25° C. for 1 h. LCMS indicated that the starting material was completely consumed, and the desired mass was detected. The mixture was concentrated under vacuum to give the crude product 1-(2-(benzyloxy)phenyl)cyclopropanamine (100 mg, HCl salt) as a white solid. M+H⁺=240.1 (LCMS).

### Step 5: N-(1-(2-(Benzyloxy)phenyl)cyclopropyl)-5-(2-(dimethylamino)ethoxy)-2-methyl benzamide (Compound 327)

To a solution of 1-(2-(benzyloxy)phenyl)cyclopropanamine (100 mg, 418 μmol, 1.0 eq) and 5-(2-(dimethylamino)ethoxy)-2-methylbenzoic acid (187 mg, 836 μmol, 2.0 eq) in DMF (4.0 mL) were added TEA (127 mg, 1.25 mmol, 174 μL, 3.0 eq), EDCI (96.1 mg, 501 μmol, 1.2 eq) and HOBt (67.8 mg, 501 μmol, 1.2 eq). The mixture was stirred at 25° C. for 3 h. LCMS indicated that the starting material was completely consumed, and the desired mass was detected. The reaction mixture was poured into H₂O (4.0 mL) and extracted with EtOAc (2.0 mL×3). The combined organic layers were dried over Na₂SO₄, filtered, and concentrated under vacuum to give a residue which was purified by preparative HPLC (Phenomenex Luna C18 column (100×40 mm, 3 μm); flow rate: 50 mL/min; gradient: 20%-60% B over 8 min; mobile phase A: 0.2% aqueous FA, mobile phase B: acetonitrile). N-(1-(2-(Benzyloxy)phenyl)cyclopropyl)-5-(2-(dimethyl amino)ethoxy)-2-methylbenzamide (60.3 mg, 123 μmol, 30% yield, FA salt) was obtained as a white solid. M+H⁺=445.1 (LCMS); 1H NMR (400 MHZ, CDCl₃) δ 8.42 (s, 1H), 7.62 (dd, J=1.4, 7.4 Hz, 1H), 7.38 (br d, J=1.0 Hz, 2H), 7.33 (br d, J=7.0 Hz, 3H), 7.25 (br d, J=1.1 Hz, 1H), 7.08-6.89 (m, 3H), 6.85-6.76 (m, 2H), 6.68 (s, 1H), 5.15 (s, 2H), 4.07 (t, J=5.3 Hz, 2H), 2.93 (t, J=5.3 Hz, 2H), 2.48 (s, 6H), 2.15 (s, 3H), 1.25 (br d, J=4.9 Hz, 4H).

### Example 191: N-(1-(3-(Benzyloxy)phenyl)cyclopropyl)-5-(2-(dimethylamino)ethoxy)-2-methylbenzamide (Compound 308)

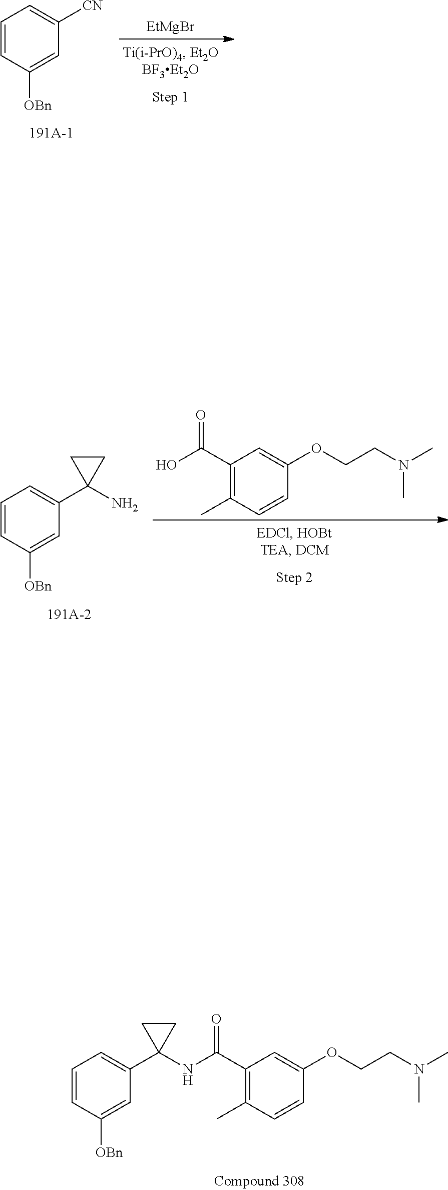 [SMILES: "EtMgBr" ; N#Cc1cccc(OCc2ccccc2)c1 ; NC1(c2cccc(OCc3ccccc3)c2)CC1 ; Cc1ccc(OCCN(C)C)cc1C(=O)O ; Cc1ccc(OCCN(C)C)cc1C(=O)NC1(c2cccc(OCc3ccccc3)c2)CC1]

### Step 1: 1-(3-(Benzyloxy)phenyl)cyclopropanamine (191A-2)

To a mixture of 3-(benzyloxy)benzonitrile (300 mg, 1.43 mmol, 1.0 eq) in anhydrous Et₂O (20 mL) was degassed and purged with N₂ three times. The mixture was stirred with a stirrer at −78° C. To this mixture was added Ti(i-PrO)₄ (448 mg, 1.58 mmol, 470 μL, 1.1 eq) slowly, and then EtMgBr (3 M in Et₂O, 1.05 mL, 2.2 eq) was added dropwise to maintain the temperature between −78° C. and −75° C. over 5 min under a N₂ atmosphere. After the addition was complete, the resulting mixture was stirred at the same temperature for 10 min and warmed to 20° C. over 1 h. BF₃·Et₂O (407 mg, 2.87 mmol, 350 μL, 2.0 eq) was added slowly with no obvious temperature change. The mixture was stirred at 20° C. for another 1 h. TLC indicated that the starting material was completely consumed, and a main spot was detected. The reaction mixture was added into a mixture of HCl (1 M aqueous) (20 mL) and MTBE (20 mL), and extracted with MTBE (20 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (20 mL×2). The combined organic layers were dried over Na₂SO₄, filtered, and concentrated under vacuum to give the crude product 1-(3-(benzyloxy)phenyl)cyclopropanamine (150 mg) as a brown solid, which was used in the next step without any further purification. M+H⁺=240.1 (LCMS).

### Step 2: N-(1-(3-(Benzyloxy)phenyl)cyclopropyl)-5-(2-(dimethylamino)ethoxy)-2-methyl benzamide (Compound 308)

To a solution of 1-(3-(benzyloxy)phenyl)cyclopropanamine (57.0 mg, 239 μmol, 1.0 eq) and 5-(2-(dimethylamino)ethoxy)-2-methylbenzoic acid (53.3 mg, 239 μmol, 1.0 eq) in DCM (2.0 mL) were added TEA (48.4 mg, 0.48 mmol, 66.5 μL, 2 eq), EDCI (68.7 mg, 0.36 mmol, 1.5 eq) and HOBt (48.4 mg, 0.36 mmol, 1.5 eq). The mixture was stirred at 20° C. for 2 h. LCMS indicated that the starting material was completely consumed, and the desired mass was detected. The reaction mixture was poured into H₂O (2.0 mL) and extracted with DCM (2.0 mL×2). The combined organic layers were dried over Na₂SO₄, filtered, and concentrated under vacuum to give a residue which was purified by preparative HPLC (Phenomenex Gemini C18 column (80×40 mm, 3 μm); flow rate: 40 mL/min; gradient: 10%-40% B over 7 min; mobile phase A: 0.04% aqueous HCl, mobile phase B: acetonitrile). N-(1-(3-(Benzyloxy)phenyl)cyclopropyl)-5-(2-(dimethylamino)ethoxy)-2-methylbenzamide (4.00 mg, 8.32 μmol, 4% yield) was obtained as a white gum. M+H⁺=445.1 (LCMS); ¹H NMR (400 MHz, DMSO-d₆) δ 8.93 (s, 1H), 7.46-7.42 (m, 2H), 7.42-7.36 (m, 2H), 7.36-7.31 (m, 1H), 7.24-7.16 (m, 2H), 7.03-6.92 (m, 2H), 6.90-6.75 (m, 3H), 5.08 (s, 2H), 4.47-4.23 (m, 2H), 3.49 (br d, J=1.8 Hz, 2H), 2.85 (d, J=4.9 Hz, 6H), 2.29-2.20 (m, 3H), 1.24 (br d, J=1.8 Hz, 4H).

### Example 192: N-(1-(4-(Benzyloxy)phenyl)cyclopropyl)-5-(2-(dimethylamino)ethoxy)-2-methylbenzamide (Compound 307)

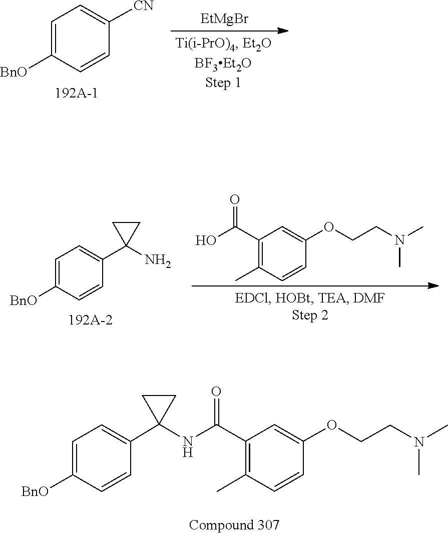 [SMILES: "EtMgBr" ; N#Cc1ccc(OCc2ccccc2)cc1 ; NC1(c2ccc(OCc3ccccc3)cc2)CC1 ; Cc1ccc(OCCN(C)C)cc1C(=O)O ; Cc1ccc(OCCN(C)C)cc1C(=O)NC1(c2ccc(OCc3ccccc3)cc2)CC1]

### Step 1: 1-(4-(Benzyloxy)phenyl)cyclopropanamine (192A-2)

A mixture of 4-(benzyloxy)benzonitrile (1.00 g, 4.78 mmol, 1.0 eq) in anhydrous Et₂O (50 mL) was degassed and purged with N₂ three times. To this mixture was added Ti(i-PrO)₄ (1.36 g, 4.78 mmol, 1.41 mL, 1.0 eq) slowly at −78° C. and then EtMgBr (3 M in Et₂O, 3.50 mL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. BF₃·Et₂O (1.36 g, 9.59 mmol, 1.18 mL, 2.0 eq) was added slowly with no obvious temperature change. The mixture was stirred at 20° C. for another 1 h. TLC indicated that the starting material was completely consumed. The reaction mixture was added into a mixture of HCl (1 M aqueous) (30 mL) and MTBE (30 mL) and extracted with MTBE (30 mL×4). The aqueous layer was basified to pH 8 using NaOH (2 M aqueous) and a precipitate was formed. The mixture was filtered through a pad of Celite and the slurry was washed with DCM several times. The combined filtrate was extracted with DCM (50 mL×4). The combined organic layers were dried over Na₂SO₄, filtered, and concentrated under vacuum to give a residue which was purified by flash silica gel chromatography using a gradient of EtOAc/petroleum ether from 0/1 to 1/1. 1-(4-(Benzyloxy)phenyl)cyclopropanamine (400 mg, 1.67 mmol, 35% yield) was obtained as a yellow oil. M+H⁺=240.2 (LCMS).

### Step 2: N-(1-(4-(Benzyloxy)phenyl)cyclopropyl)-5-(2-(dimethylamino)ethoxy)-2-methylbenzamide (Compound 307)

To a solution of 5-(2-(dimethylamino)ethoxy)-2-methylbenzoic acid (112 mg, 501 μmol, 1.2 eq) in DMF (2.0 mL) were added 1-(4-(benzyloxy)phenyl)cyclopropanamine (100 mg, 418 μmol, 1.0 eq), TEA (127 mg, 1.25 mmol, 174 μL, 3.0 eq), EDCI (120 mg, 627 μmol, 1.5 eq) and HOBt (84.7 mg, 627 μmol, 1.5 eq). The mixture was stirred at 20° C. for 16 h. LCMS indicated that the starting material was completely consumed, and the desired mass was detected. The reaction mixture was poured into H₂O (5.0 mL) and extracted with EtOAc (5.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 20%-45% B over 8 min; mobile phase A: 0.04% aqueous HCl, mobile phase B: acetonitrile). N-(1-(4-(Benzyloxy)phenyl)cyclopropyl)-5-(2-(dimethylamino)ethoxy)-2-methylbenzamide (22.2 mg, 50.0 μmol, 12% yield, HCl salt) was obtained as a yellow solid. M+H⁺=445.2 (LCMS); 1H NMR (400 MHZ, DMSO-d₆) δ 10.73-10.55 (m, 1H), 8.96 (s, 1H), 7.45-7.30 (m, 5H), 7.21-7.14 (m, 3H), 6.99-6.90 (m, 4H), 5.08 (s, 2H), 4.36 (br t, J=4.8 Hz, 2H), 3.53-3.44 (m, 2H), 2.82 (d, J=4.9 Hz, 6H), 2.25-2.19 (m, 3H), 1.24-1.10 (m, 4H).

### Example 193: N-(1-([1,1′-Biphenyl]-3-yl)cyclopropyl)-5-(2-(dimethylamino)ethoxy)-2-methylbenzamide (Compound 291)

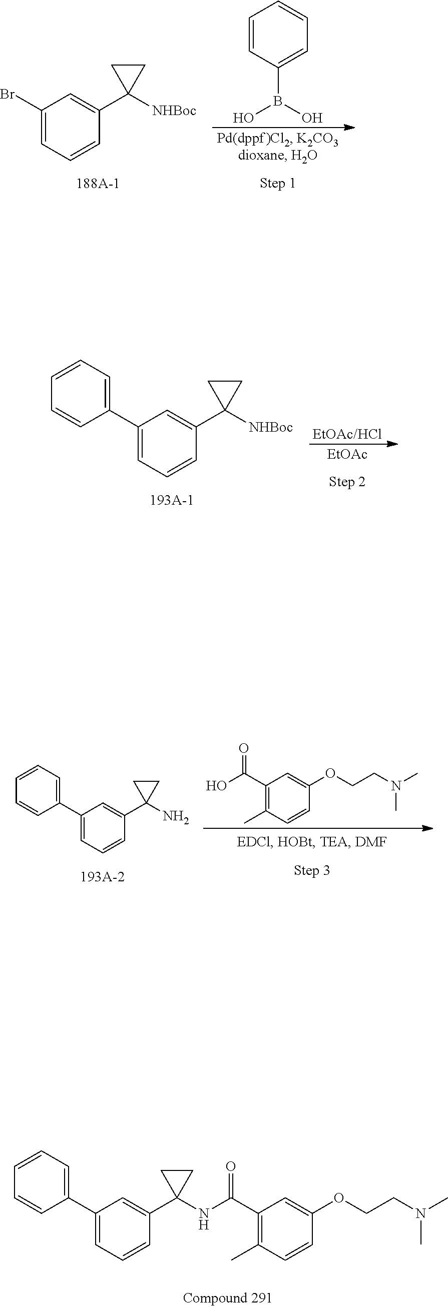

### Step 1: tert-Butyl(1-([1,1′-biphenyl]-3-yl)cyclopropyl)carbamate (193A-1)

A mixture of tert-butyl N-[1-(3-bromophenyl)cyclopropyl]carbamate (150 mg, 480 μmol, 1.0 eq), phenylboronic acid (146 mg, 1.20 mmol, 2.5 eq), Pd(dppf)Cl₂ (35.2 mg, 48.1 μmol, 0.1 eq) and K₂CO₃ (166 mg, 1.20 mmol, 2.5 eq) in a mixture of dioxane (6.0 mL) and H₂O (1.5 mL) was degassed and purged with N₂ three times. The mixture was stirred at 80° C. for 16 h under a N₂ atmosphere. LCMS indicated that the starting material was completely consumed, and the desired mass was detected. The reaction mixture was poured into H₂O (4.0 mL) and extracted with EtOAc (4.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/4. The crude product tert-butyl(1-([1,1′-biphenyl]-3-yl)cyclopropyl)carbamate (180 mg) was obtained as a white solid.

### Step 2: 1-([1,1′-Biphenyl]-3-yl)cyclopropanamine (193A-2)

To a solution of tert-butyl(1-([1,1′-biphenyl]-3-yl)cyclopropyl)carbamate (100 mg, 323 μmol, 1.0 eq) in EtOAc (1.0 mL) was added HCl/EtOAc (4 M, 3.0 mL). The mixture was stirred at 20° C. for 30 min. LCMS indicated that the starting material was completely consumed, and the desired mass was detected. The mixture was concentrated under vacuum at 30° C. to give the crude product 1-([1,1′-biphenyl]-3-yl)cyclopropanamine (120 mg, HCl salt) as a white solid, which was used in the next step without any further purification. M+H⁺=210.1 (LCMS).

### Step 3: N-(1-([1,1′-Biphenyl]-3-yl)cyclopropyl)-5-(2-(dimethylamino)ethoxy)-2-methyl benzamide (Compound 291)

To a solution of 1-([1,1′-biphenyl]-3-yl)cyclopropanamine (120 mg, 573 μmol, 1.0 eq, HCl salt) and 5-(2-(dimethylamino)ethoxy)-2-methylbenzoic acid (154 mg, 688 μmol, 1.2 eq) in DMF (4.0 mL) were added EDCI (165 mg, 860 μmol, 1.5 eq), HOBt (116 mg, 860 μmol, 1.5 eq) and TEA (116 mg, 1.15 mmol, 160 μL, 2.0 eq). The mixture was stirred at 20° C. for 16 h. LCMS indicated that the starting material was completely consumed, and the desired mass was detected. The reaction mixture was poured into H₂O (4.0 mL) and extracted with EtOAc (4.0 mL×3). The combined organic layers were dried over Na₂SO₄, filtered, and concentrated under vacuum to give a residue which was purified by preparative HPLC (Phenomenex C18 column (80×40 mm, 3 μm); flow rate: 60 mL/min; gradient: 40%-60% B over 8 min; mobile phase A: 10 mM aqueous NH₄HCO₃, mobile phase B: acetonitrile). N-(1-([1,1′-Biphenyl]-3-yl)cyclopropyl)-5-(2-(dimethyl amino)ethoxy)-2-methylbenzamide (24.5 mg, 55.5 μmol, 10% yield) was obtained as an light yellow gum. M+H⁺=415.1 (LCMS); ¹H NMR (400 MHZ, DMSO-d₆) δ 8.99 (s, 1H), 7.67-7.59 (m, 1H), 7.67-7.56 (m, 1H), 7.51-7.31 (m, 6H), 7.21 (br d, J=7.6 Hz, 1H), 7.14 (d, J=8.3 Hz, 1H), 6.97-6.88 (m, 2H), 4.07-4.02 (m, 2H), 2.64-2.58 (m, 1H), 2.61 (t, J=5.8 Hz, 1H), 2.27-2.18 (m, 9H), 1.37-1.25 (m, 4H).

### Example 194: N-(1-(3-(Cyclopent-1-en-1-yl)phenyl)cyclopropyl)-5-(2-(dimethylamino)ethoxy)-2-methylbenzamide (Compound 264)

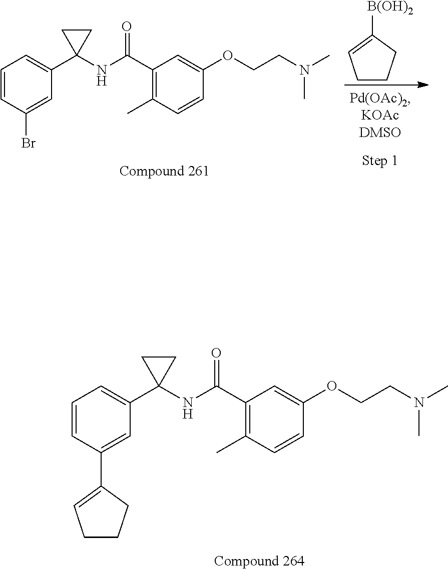

### Step 1: N-(1-(3-(Cyclopent-1-en-1-yl)phenyl)cyclopropyl)-5-(2-(dimethylamino)ethoxy)-2-methylbenzamide (Compound 264)

To a stirred solution of N-(1-(3-(cyclopent-1-en-1-yl)phenyl)cyclopropyl)-5-(2-(dimethyl amino)ethoxy)-2-methylbenzamide (170 mg, 407 μmol, 1.0 eq) and cyclopent-1-en-1-ylboronic acid (103 mg, 530 μmol, 1.3 eq) in DMSO (5.0 mL) were added Pd(OAc) 2 (9.15 mg, 40.7 μmol, 0.1 eq) and KOAc (120 mg, 1.22 mmol, 3.0 eq) under a N₂ atmosphere. The mixture was stirred at 80° C. for 16 h. LCMS indicated that the starting material was completely consumed, and the desired mass was detected. The reaction mixture was allowed to cool to room temperature, poured into H₂O (5.0 mL) and extracted with EtOAc (4.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 25%-65% B over 8 min; mobile phase A: 10 mM aqueous NH₄HCO₃, mobile phase B: acetonitrile). N-(1-(3-(Cyclopent-1-en-1-yl)phenyl)cyclopropyl)-5-(2-(dimethylamino)ethoxy)-2-methylbenzamide (120 mg, 297 μmol, 73% yield) was obtained as a yellow solid. M+H⁺=405.1 (LCMS); ¹H NMR (400 MHz, DMSO-d₆) δ 8.94 (s, 1H), 7.33 (s, 1H), 7.29-7.22 (m, 2H), 7.14 (d, J=9.3 Hz, 1H), 7.05 (br d, J=7.0 Hz, 1H), 6.95-6.89 (m, 2H), 6.24 (br s, 1H), 4.05 (t, J=5.8 Hz, 2H), 2.69-2.60 (m, 4H), 2.47 (br d, J=2.1 Hz, 2H), 2.24 (s, 3H), 2.22 (s, 6H), 2.01-1.91 (m, 2H), 1.25 (s, 4H).

### Example 195: N-(1-(3-Cyclopentylphenyl)cyclopropyl)-5-(2-(dimethylamino)ethoxy)-2-methylbenzamide (Compound 276)

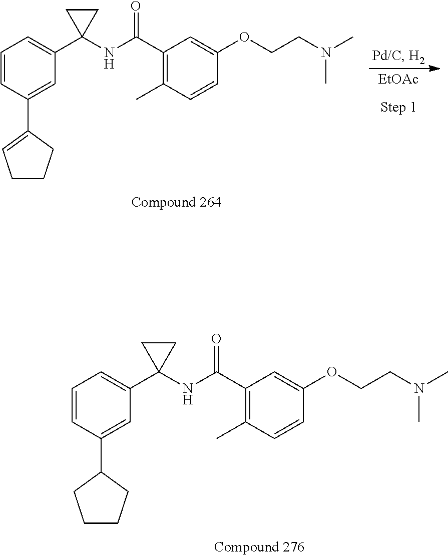 [SMILES: "EtOAc" ; Cc1ccc(OCCN(C)C)cc1C(=O)NC1(c2cccc(C3=CCCC3)c2)CC1 ; "Pd/C, H2" ; Cc1ccc(OCCN(C)C)cc1C(=O)NC1(c2cccc(C3CCCC3)c2)CC1]

### Step 1: N-(1-(3-Cyclopentylphenyl)cyclopropyl)-5-(2-(dimethylamino)ethoxy)-2-methylbenzamide (Compound 276)

To a solution of N-(1-(3-(cyclopent-1-en-1-yl)phenyl)cyclopropyl)-5-(2-(dimethylamino) ethoxy)-2-methylbenzamide (70.0 mg, 173 μmol, 1.0 eq) in EtOAc (7.0 mL) was added 10% palladium on carbon (10.0 mg) at 20° C. for 2 h under a H₂ atmosphere (15 psi). LCMS indicated that the starting material was completely consumed, and the desired mass was detected. The reaction mixture was filtered through a pad of Celite and the filtrate was concentrated under vacuum. N-(1-(3-Cyclopentylphenyl)cyclopropyl)-5-(2-(dimethylamino)ethoxy)-2-methylbenzamide (38.0 mg, 89.1 μmol, 51% yield) was obtained as a yellow solid. M+H⁺=407.1 (LCMS); ¹H NMR (400 MHZ, DMSO-d₆) δ 8.93 (br s, 1H), 7.23-7.11 (m, 3H), 7.07-7.01 (m, 1H), 6.99-6.88 (m, 3H), 4.11-3.99 (m, 2H), 3.06-2.79 (m, 1H), 2.62 (br d, J=5.5 Hz, 2H), 2.34-2.17 (m, 9H), 1.99 (br d, J=5.9 Hz, 2H), 1.81-1.46 (m, 6H), 1.23 (br s, 4H).

### Example 196: 5-(2-(Dimethylamino)ethoxy)-2-methyl-N-(1-(2′,3′,4′,5′-tetrahydro-[1,1′-biphenyl]-3-yl)cyclopropyl)benzamide (Compound 338)

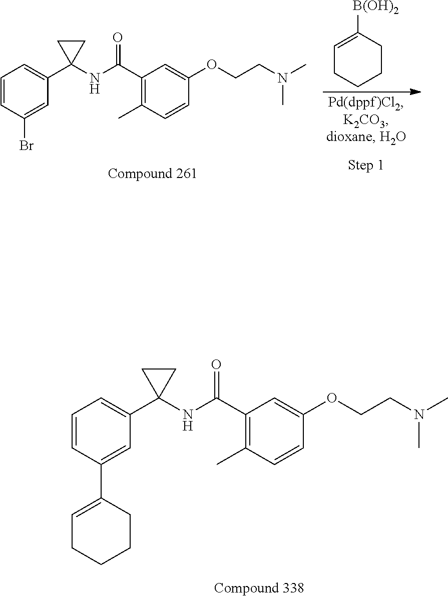

### Step 1: 5-(2-(Dimethylamino)ethoxy)-2-methyl-N-(1-(2′,3′,4′,5′-tetrahydro-[1,1′-biphenyl]-3-yl)cyclopropyl)benzamide (Compound 338)

To a stirred solution of N-(1-(3-bromophenyl)cyclopropyl)-5-(2-(dimethylamino)ethoxy)-2-methylbenzamide (80.0 mg, 192 μmol, 1.0 eq) and cyclohex-1-en-1-ylboronic acid (29.0 mg, 230 μmol, 1.2 eq) in a mixture of dioxane (1.0 mL) and H₂O (1.0 mL) were added K₂CO₃ (79.5 mg, 575 μmol, 3.0 eq) and Pd(dppf)Cl₂ (14.0 mg, 19.2 μmol, 0.1 eq). The mixture was stirred at 90° C. for 5 h. LCMS indicated that the starting material was completely consumed, and the desired mass was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 5%-45% B over 8 min; mobile phase A: 0.04% aqueous HCl, mobile phase B: acetonitrile). 5-(2-(Dimethylamino)ethoxy)-2-methyl-N-(1-(2′,3′,4′,5′-tetrahydro-[1,1′-biphenyl]-3-yl)cyclopropyl)benzamide (44.3 mg, 105 μmol, 55% yield, HCl salt) was obtained as a white solid. M+H⁺=419.1 (LCMS); ¹H NMR (400 MHZ, DMSO-d₆) δ 10.65 (br s, 1H), 8.99 (br s, 1H), 7.33-7.16 (m, 4H), 7.08 (br d, J=6.4 Hz, 1H), 7.02-6.93 (m, 2H), 6.11 (br s, 1H), 4.37 (br s, 2H), 3.54-3.48 (m, 2H), 2.83 (br s, 6H), 2.35 (br s, 2H), 2.25 (br s, 3H), 2.17 (br s, 2H), 1.72 (br s, 2H), 1.61 (br d, J=4.6 Hz, 2H), 1.25 (br s, 4H).

### Example 197: 5-(2-(Dimethylamino)ethoxy)-2-methyl-N-(1-(2-(pyridin-4-yl)phenyl)cyclopropyl)benzamide (Compound 266)

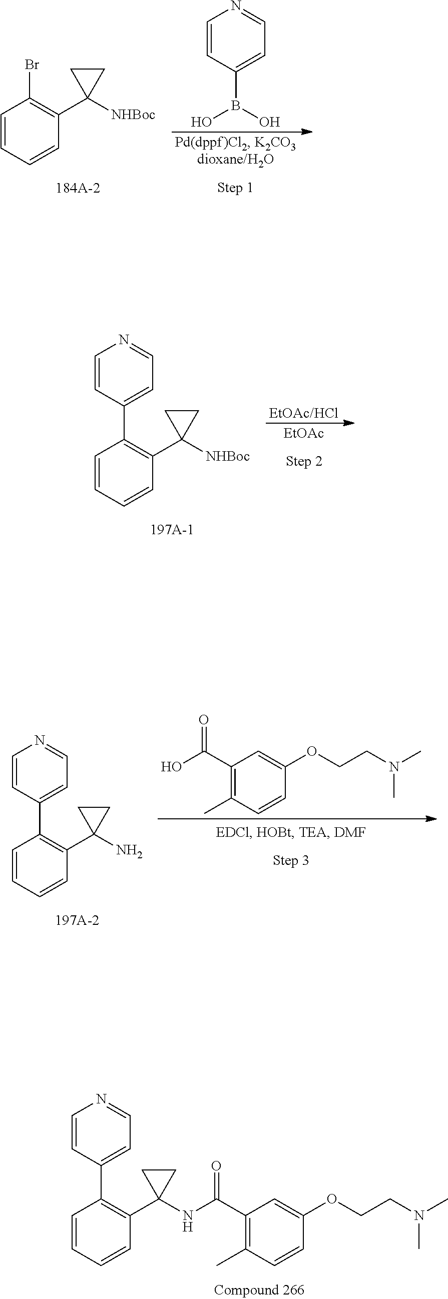

### Step 1: tert-Butyl(1-(2-(pyridin-4-yl)phenyl)cyclopropyl)carbamate (197A-1)

A mixture of pyridin-4-ylboronic acid (295 mg, 2.40 mmol, 2.5 eq), tert-butyl(1-(2-bromophenyl)cyclopropyl)carbamate (300 mg, 961 μmol, 1.0 eq), K₂CO₃ (332 mg, 2.40 mmol, 2.5 eq), Pd(dppf)Cl₂ (70.3 mg, 96.1 μmol, 0.1 eq) in a mixture of dioxane (12 mL) and H₂O (3.0 mL) was stirred at 80° C. for 16 h under a N₂ atmosphere. LCMS indicated that the starting material was completely consumed, and the desired mass was detected. The reaction mixture was allowed to cool to room temperature, poured into H₂O (10 mL) and extracted with DCM (10 mL×5). The combined organic layers were dried over Na₂SO₄, filtered, and concentrated under vacuum to give a residue which was purified by flash silica gel chromatography using a gradient of EtOAc/petroleum ether from 0/1 to 1/0. tert-Butyl(1-(2-(pyridin-4-yl)phenyl)cyclopropyl)carbamate (80.0 mg, 258 μmol, 27% yield) was obtained as a yellow solid. M+H⁺=311.1 (LCMS).

### Step 2: 1-(2-(Pyridin-4-yl)phenyl)cyclopropanamine (197A-2)

To a solution of tert-butyl(1-(2-(pyridin-4-yl)phenyl)cyclopropyl)carbamate (80.0 mg, 258 μmol, 1.0 eq) in EtOAc (1.0 mL) was added HCl/EtOAc (4 M, 3.0 mL). The resulting mixture was stirred at 25° C. for 1 h. LCMS indicated that the starting material was completely consumed, and the desired mass was detected. The reaction mixture was concentrated under vacuum to give 1-(2-(pyridin-4-yl)phenyl)cyclopropanamine (50.0 mg, 203 μmol, 79% yield, HCl salt) as a yellow solid. M+H⁺=211.1 (LCMS).

### Step 3: 5-(2-(Dimethylamino)ethoxy)-2-methyl-N-(1-(2-(pyridin-4-yl)phenyl)cyclopropyl)benzamide (Compound 266)

To a solution of 5-(2-(dimethylamino)ethoxy)-2-methylbenzoic acid (54.5 mg, 244 μmol, 1.2 eq), 1-(2-(pyridin-4-yl)phenyl)cyclopropanamine (50.0 mg, 203 μmol, 1.0 eq, HCl salt) in DMF (3.0 mL) were added TEA (61.8 mg, 610 μmol, 84.9 μL, 3.0 eq), EDCI (46.8 mg, 244 μmol, 1.2 eq) and HOBt (33.0 mg, 244 μmol, 1.2 eq). The mixture was stirred at 25° C. for 16 h. LCMS indicated that the starting material was completely consumed, and the desired mass was detected. The reaction mixture was poured into H₂O (10 mL) and extracted with DCM (5.0 mL×5). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 1%-20% B over 8 min; mobile phase A: 0.2% aqueous FA, mobile phase B: acetonitrile). 5-(2-(Dimethylamino)ethoxy)-2-methyl-N-(1-(2-(pyridin-4-yl)phenyl)cyclopropyl)benzamide (49.8 mg, 108 μmol, 53% yield, FA salt) was obtained as a yellow gum. M+H⁺=416.1 (LCMS); ¹H NMR (400 MHZ, DMSO-d₆) δ 8.60 (d, J=5.9 Hz, 2H), 8.25 (s, 1H), 8.17 (s, 1H), 7.90 (dd, J=1.0, 7.5 Hz, 1H), 7.52 (d, J=5.9 Hz, 2H), 7.38 (dtd, J=1.4, 7.4, 18.6 Hz, 2H), 7.18 (dd, J=1.3, 7.3 Hz, 1H), 7.05 (d, J=8.4 Hz, 1H), 6.83 (dd, J=2.6, 8.4 Hz, 1H), 6.48 (d, J=2.6 Hz, 1H), 4.00 (t, J=5.6 Hz, 2H), 2.68 (t, J=5.5 Hz, 2H), 2.27 (s, 6H), 2.08 (s, 3H), 1.02 (s, 4H).

### Example 198: 5-(2-(Dimethylamino)ethoxy)-2-methyl-N-(1-(3-(thiophen-2-yl)phenyl)cyclopropyl)benzamide (Compound 355)

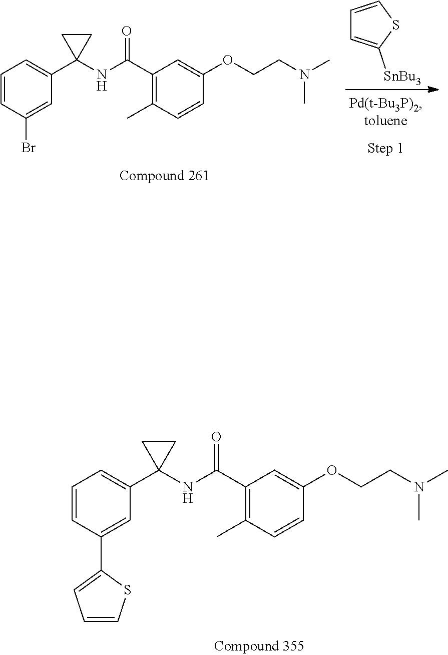

### Step 1: 5-(2-(Dimethylamino)ethoxy)-2-methyl-N-(1-(3-(thiophen-2-yl)phenyl)cyclopropyl)benzamide (Compound 355)

To a solution of N-(1-(3-bromophenyl)cyclopropyl)-5-(2-(dimethylamino)ethoxy)-2-methylbenzamide (40.0 mg, 95.9 μmol, 1.0 eq) in toluene (3.0 mL) were added tributyl(thiophen-2-yl) stannane (53.7 mg, 144 μmol, 45.5 μL, 1.5 eq) and Pd(t-Bu₃P)₂ (4.90 mg, 9.58 μmol, 0.1 eq). The mixture was stirred at 90° C. for 3 h under a N₂ atmosphere. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 20%-50% B over 8 min; mobile phase A: 0.2% aqueous FA, mobile phase B: acetonitrile). 5-(2-(Dimethylamino)ethoxy)-2-methyl-N-(1-(3-(thiophen-2-yl)phenyl)cyclopropyl)benzamide (8.40 mg, 18.0 μmol, 19% yield, FA salt) was obtained as a yellow oil. M+H⁺=421.1 (LCMS); ¹H NMR (400 MHZ, DMSO-d₆) δ 9.01 (s, 1H), 8.19 (s, 1H), 7.56-7.51 (m, 2H), 7.51-7.46 (m, 2H), 7.35 (t, J=7.8 Hz, 1H), 7.18-7.07 (m, 3H), 6.97-6.90 (m, 2H), 4.07 (t, J=5.8 Hz, 2H), 2.64 (t, J=5.8 Hz, 2H), 2.30-2.20 (m, 9H), 1.30 (br d, J=3.8 Hz, 4H).

### Example 199: 5-(2-(Dimethylamino)ethoxy)-2-methyl-N-(1-(3-(thiophen-3-yl)phenyl)cyclopropyl)benzamide (Compound 325)

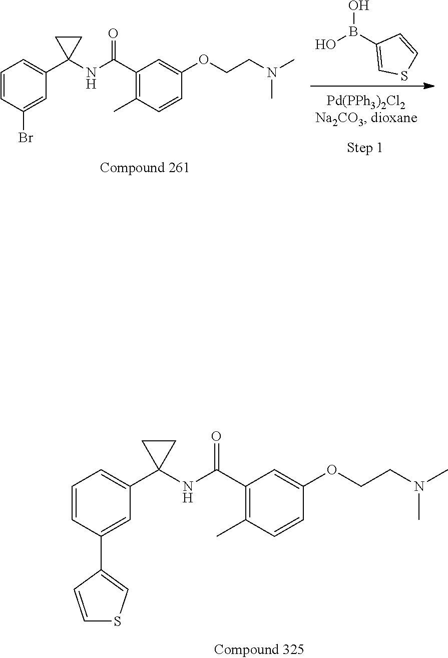 [SMILES: Cc1ccc(OCCN(C)C)cc1C(=O)NC1(c2cccc(Br)c2)CC1 ; OB(O)c1ccsc1 ; Cc1ccc(OCCN(C)C)cc1C(=O)NC1(c2cccc(-c3ccsc3)c2)CC1]

### Step 1: 5-(2-(Dimethylamino)ethoxy)-2-methyl-N-(1-(3-(thiophen-3-yl)phenyl)cyclopropyl)benzamide (Compound 325)

A mixture of N-(1-(3-bromophenyl)cyclopropyl)-5-(2-(dimethylamino)ethoxy)-2-methylbenzamide (50.0 mg, 120 μmol, 1.0 eq), thiophen-3-ylboronic acid (15.3 mg, 120 μmol, 1.0 eq) and Na₂CO₃ (38.1 mg, 359 μmol, 3.0 eq) in dioxane (2.0 mL) was degassed and purged with N₂ three times. To the mixture was added Pd(PPh₃)₂Cl₂ (8.41 mg, 12.0 μmol, 0.1 eq). The mixture was stirred at 100° C. for 3 h. LCMS indicated that the starting material was completely consumed, and the desired mass was detected. The reaction mixture was allowed to cool to room temperature, poured into H₂O (3.0 mL) and extracted with EtOAc (2.0 mL×2). The combined organic layers were dried over Na₂SO₄, filtered, and concentrated under vacuum to give a residue which was purified by preparative HPLC (Phenomenex Gemini C18 column (80×30 mm, 3 μm); flow rate: 25 mL/min; gradient: 15%-40% B over 8 min; mobile phase A: 0.04% aqueous HCl, mobile phase B: acetonitrile). 5-(2-(Dimethylamino)ethoxy)-2-methyl-N-(1-(3-(thiophen-3-yl)phenyl)cyclopropyl)benzamide (7.50 mg, 16.4 μmol, 14% yield, HCl salt) was obtained as a yellow solid. M+H⁺=421.1 (LCMS); ¹H NMR (400 MHZ, DMSO-d₆) δ 10.09-10.38 (m, 1H), 9.02 (s, 1H), 7.83 (d, J=1.47 Hz, 1H), 7.62-7.70 (m, 1H), 7.44-7.57 (m, 3H), 7.30-7.40 (m, 1H), 7.16-7.25 (m, 2H), 6.94-7.05 (m, 2H), 4.35 (br t, J=4.71 Hz, 2H), 3.50 (br d, J=4.52 Hz, 2H), 2.76-2.93 (m, 6H), 2.26 (s, 3H), 1.23-1.45 (m, 4H).

### Example 200: 5-(Azetidin-3-ylamino)-2-methyl-N-(1-(3-(thiophen-2-yl)phenyl)cyclopropyl)benzamide (Compound 169)

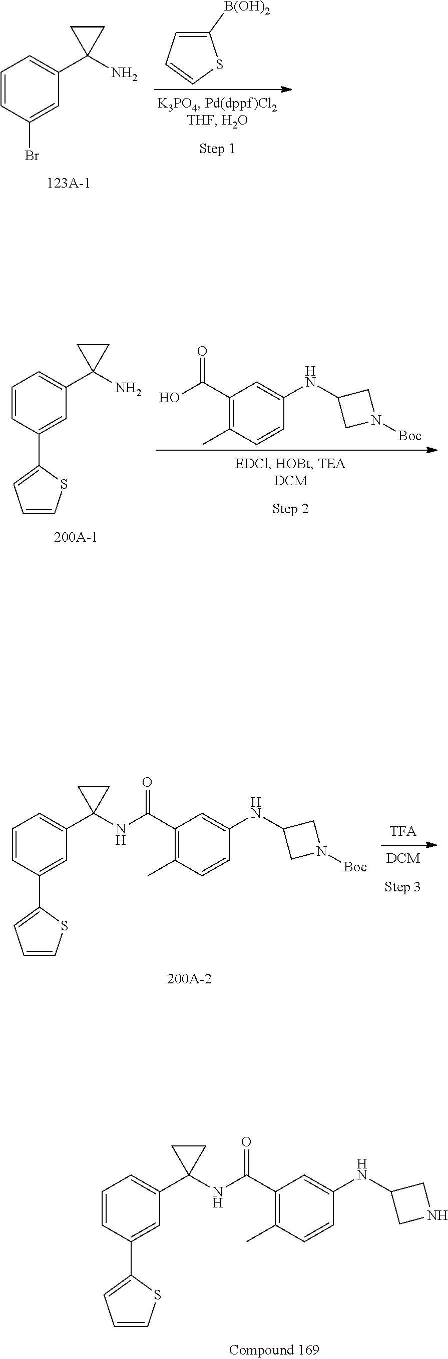 [SMILES: NC1(c2cccc(Br)c2)CC1 ; OB(O)c1cccs1 ; NC1(c2cccc(-c3cccs3)c2)CC1 ; Cc1ccc(NC2CN(C(=O)OC(C)(C)C)C2)cc1C(=O)O ; Cc1ccc(NC2CN(C(=O)OC(C)(C)C)C2)cc1C(=O)NC1(c2cccc(-c3cccs3)c2)CC1 ; Cc1ccc(NC2CNC2)cc1C(=O)NC1(c2cccc(-c3cccs3)c2)CC1]

### Step 1: 1-(3-(Thiophen-2-yl)phenyl)cyclopropanamine (200A-1)

To a solution of 1-(3-bromophenyl)cyclopropanamine (300 mg, 1.41 mmol, 1.0 eq) in a mixture of THF (10 mL) and H₂O (2.5 mL) were added thiophen-2-ylboronic acid (253 mg, 1.98 mmol, 1.4 eq), K₃PO₄ (901 mg, 4.24 mmol, 3.0 eq) and Pd(dppf)Cl₂ (46.1 mg, 70.7 μmol, 0.1 eq). The mixture was degassed and purged with N₂ three times, then stirred at 80° C. for 16 h under a N₂ atmosphere. TLC indicated that the starting material was completely consumed. The reaction mixture was allowed to cool to room temperature, poured into H₂O (5.0 mL) and extracted with EtOAc (5.0 mL×2). The combined organic layers were dried over Na₂SO₄, filtered, and concentrated under vacuum to give a residue which was purified by preparative TLC (EtOAc/petroleum ether=1/2, R_{f}=0.2). 1-(3-(Thiophen-2-yl)phenyl)cyclopropanamine (100 mg, 464 μmol, 33% yield) was obtained as a yellow oil. M+H⁺=216.2 (LCMS).

### Step 2: tert-Butyl 3-((4-methyl-3-((1-(3-(thiophen-2-yl)phenyl)cyclopropyl)carbamoyl)phenyl)amino)azetidine-1-carboxylate (200A-2)

To a solution of 5-((1-(tert-butoxycarbonyl)azetidin-3-yl)amino)-2-methylbenzoic acid (71.1 mg, 232 μmol, 1.0 eq) in DCM (5.0 mL) were added 1-(3-(thiophen-2-yl)phenyl)cyclopropanamine (50.0 mg, 232 μmol, 1.0 eq), TEA (70.5 mg, 697 μmol, 97.0 μL, 3.0 eq), EDCI (89.0 mg, 464 μmol, 2.0 eq) and HOBt (62.8 mg, 464 μmol, 2.0 eq). The mixture was stirred at 20° C. for 16 h. TLC indicated that the starting material was completely consumed. The reaction mixture was poured into H₂O (5.0 mL) and extracted with EtOAc (5.0 mL×2). The combined organic layers were dried over Na₂SO₄, filtered, and concentrated under vacuum to give a residue which was purified by preparative TLC (EtOAc/petroleum ether=1/2, R_{f}=0.3). tert-Butyl 3-((4-methyl-3-((1-(3-(thiophen-2-yl)phenyl)cyclopropyl)carbamoyl)phenyl)amino)azetidine-1-carboxylate (85.0 mg, 169 μmol, 73% yield) was obtained as a colorless oil. M+H⁺=504.3 (LCMS).

### Step 3: 5-(Azetidin-3-ylamino)-2-methyl-N-(1-(3-(thiophen-2-yl)phenyl)cyclopropyl)benzamide (Compound 169)

To a stirred solution of tert-butyl 3-((4-methyl-3-((1-(3-(thiophen-2-yl)phenyl)cyclopropyl) carbamoyl)phenyl)amino)azetidine-1-carboxylate (85.0 mg, 169 μmol, 1.0 eq) in DCM (8.0 mL) was added TFA (2.0 mL). The mixture was stirred at 25° C. for 1 h. LCMS indicated that the starting material was completely consumed, and the desired mass was detected. The reaction mixture was poured into H₂O (5.0 mL) and extracted with EtOAc (5.0 mL×2). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 15%-55% B over 8 min; mobile phase A: 0.1% aqueous TFA, mobile phase B: acetonitrile). 5-(Azetidin-3-ylamino)-2-methyl-N-(1-(3-(thiophen-2-yl)phenyl)cyclopropyl)benzamide (40.2 mg, 99.5 μmol, 59% yield, TFA salt) was obtained as a yellow solid. M+H⁺=404.1 (LCMS); ¹H NMR (400 MHZ, CD₃OD) δ 9.10 (s, 1H), 7.67 (s, 1H), 7.49 (d, J=7.6 Hz, 1H), 7.41-7.31 (m, 3H), 7.21 (d, J=7.7 Hz, 1H), 7.11-7.08 (m, 1H), 7.05 (d, J=8.1 Hz, 1H), 6.62-6.55 (m, 2H), 4.60-4.44 (m, 1H), 4.41-4.29 (m, 2H), 3.94 (br dd, J=6.9, 10.8 Hz, 2H), 2.26 (s, 3H), 1.37 (br d, J=3.5 Hz, 4H).

### Example 201: N-(1-([1,1′-Biphenyl]-3-yl)cyclopropyl)-5-(azetidin-3-ylamino)-2-methylbenzamide (Compound 187)

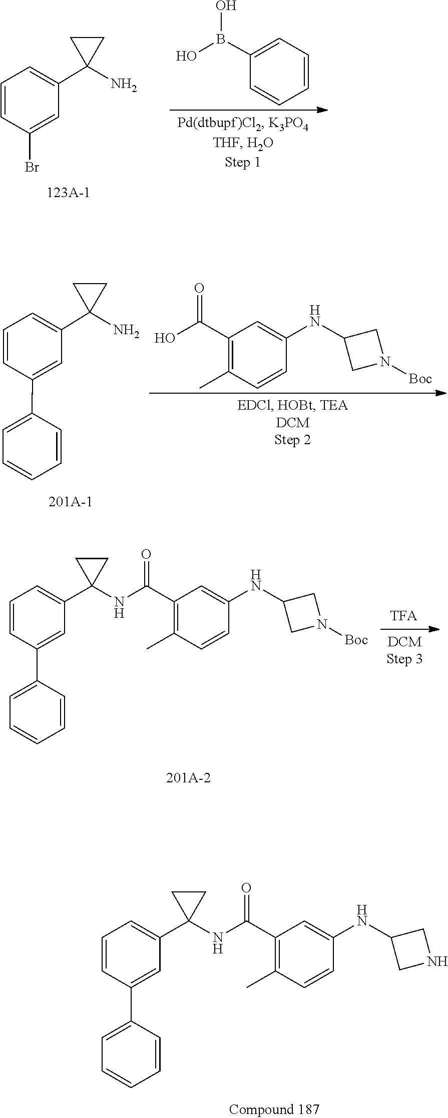 [SMILES: NC1(c2cccc(Br)c2)CC1 ; OB(O)c1ccccc1 ; NC1(c2cccc(-c3ccccc3)c2)CC1 ; Cc1ccc(NC2CN(C(=O)OC(C)(C)C)C2)cc1C(=O)O ; Cc1ccc(NC2CN(C(=O)OC(C)(C)C)C2)cc1C(=O)NC1(c2cccc(-c3ccccc3)c2)CC1 ; Cc1ccc(NC2CNC2)cc1C(=O)NC1(c2cccc(-c3ccccc3)c2)CC1]

### Step 1: 1-([1,1′-Biphenyl]-3-yl)cyclopropanamine (201A-1)

A mixture of 1-(3-bromophenyl)cyclopropanamine (200 mg, 943 μmol, 1.0 eq), phenylboronic acid (161 mg, 1.32 mmol, 1.4 eq) and potassium phosphate (601 mg, 2.83 mmol, 3.0 eq) in a mixture of H₂O (2.0 mL) and THF (8.0 mL) was degassed and purged with N₂ three times. To the mixture was added dichloro[1,1′-bis(di-t-butylphosphino) ferrocene]palladium (II)(30.7 mg, 47.2 μmol, 0.05 eq) at 25° C. The resulting mixture was stirred at 80° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (3.0 mL×3). The combined organic layers were dried over Na₂SO₄, filtered, and concentrated under vacuum to give a residue which was purified by flash silica gel chromatography using a gradient of EtOAc/petroleum ether from 1/100 to 100/1. 1-([1,1′-Biphenyl]-3-yl)cyclopropanamine (30.0 mg, 143 μmol, 15% yield) was obtained as a brown solid. M+H⁺=210.2 (LCMS); ¹H NMR (400 MHZ, CDCl₃) δ 7.60 (d, J=7.5 Hz, 2H), 7.54 (s, 1H), 7.49-7.45 (m, 2H), 7.44-7.41 (m, 2H), 7.41-7.35 (m, 2H), 1.15-1.10 (m, 2H), 1.08-1.03 (m, 2H).

### Step 2: tert-Butyl 3-((3-((1-([1,1′-biphenyl]-3-yl)cyclopropyl)carbamoyl)-4-methylphenyl)amino)azetidine-1-carboxylate (201A-2)

To a solution of 1-([1,1′-biphenyl]-3-yl)cyclopropanamine (30.0 mg, 143 μmol, 1.0 eq) and 5-((1-(tert-butoxycarbonyl)azetidin-3-yl)amino)-2-methylbenzoic acid (43.9 mg, 143 μmol, 1.0 eq) in DCM (10 mL) were added TEA (43.5 mg, 430 μmol, 59.9 μL, 3.0 eq), EDCI (55.0 mg, 287 μmol, 2.0 eq) and HOBt (38.7 mg, 287 μmol, 2.0 eq). The mixture was stirred at 20° C. for 16 h. LCMS indicated that the starting material was completely consumed, and the desired mass was detected. The reaction mixture was poured into H₂O (5.0 mL) and extracted with DCM (2.0 mL×3). The combined organic layers were dried over Na₂SO₄, filtered, and concentrated under vacuum to give a residue which was purified by preparative TLC (EtOAc/petroleum ether=1/1, R_{f}=0.4). tert-Butyl 3-((3-((1-([1,1′-biphenyl]-3-yl)cyclopropyl)carbamoyl)-4-methylphenyl)amino)azetidine-1-carboxylate (10.0 mg, 20.1 μmol, 14% yield) was obtained as a colorless oil. M+H⁺=498.4 (LCMS).

### Step 3: N-(1-([1,1′-Biphenyl]-3-yl)cyclopropyl)-5-(azetidin-3-ylamino)-2-methylbenzamide (Compound 187)

To a stirred solution of tert-butyl 3-((3-((1-([1,1′-biphenyl]-3-yl)cyclopropyl)carbamoyl)-4-methylphenyl)amino)azetidine-1-carboxylate (100 mg, 216 μmol, 1.0 eq) in DCM (8.0 mL) was added TFA (2.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 10%-40% B over 8 min; mobile phase A: 0.10% aqueous TFA, mobile phase B: acetonitrile). N-(1-([1,1′-Biphenyl]-3-yl)cyclopropyl)-5-(azetidin-3-ylamino)-2-methyl benzamide (43.1 mg, 90.3 μmol, 42% yield, TFA salt) was obtained as a white solid. M+H⁺=398.1 (LCMS); 1H NMR (400 MHZ, DMSO-d₆) δ 8.91 (s, 1H), 8.77-8.70 (m, 1H), 7.63 (d, J=7.2 Hz, 2H), 7.51-7.44 (m, 4H), 7.42-7.34 (m, 2H), 7.21 (d, J=7.7 Hz, 1H), 7.00 (d, J=7.9 Hz, 1H), 6.53-6.49 (m, 2H), 6.35 (br d, J=6.4 Hz, 1H), 4.41-4.31 (m, 1H), 4.24 (br s, 2H), 3.78 (br s, 2H), 2.17 (s, 3H), 1.37-1.31 (m, 2H), 1.30-1.24 (m, 2H).

### Example 202: 5-(Azetidin-3-ylamino)-2-methyl-N-(1-(3-(5-(pyrrolidin-1-ylmethyl)thiophen-2-yl)phenyl)cyclopropyl)benzamide (Compound 186)

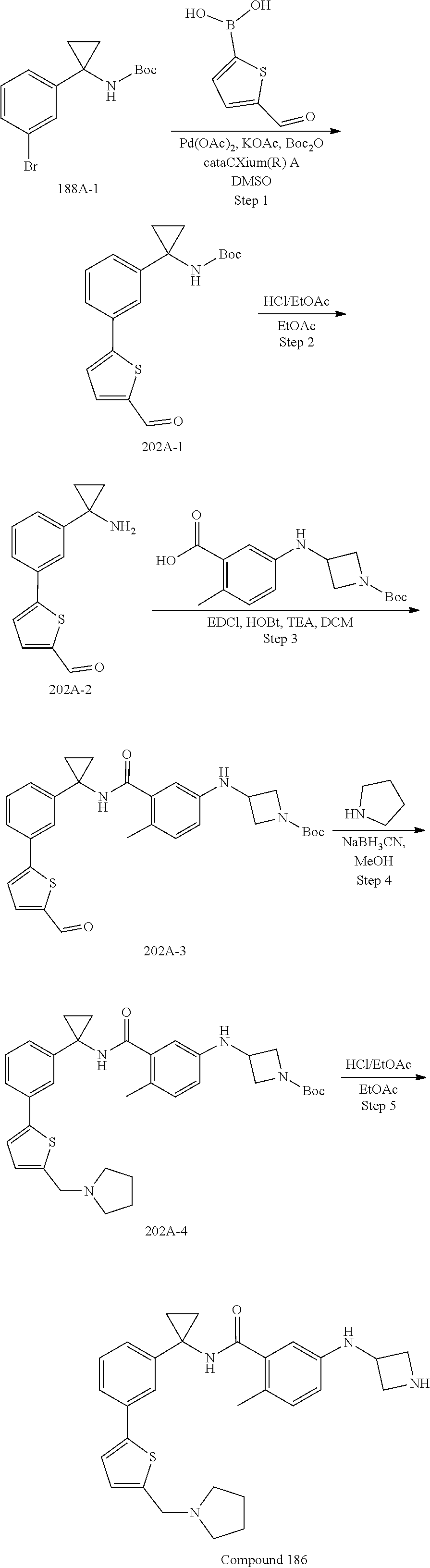

### Step 1: tert-Butyl(1-(3-(5-formylthiophen-2-yl)phenyl)cyclopropyl)carbamate (202A-1)

To a solution of tert-butyl(1-(3-bromophenyl)cyclopropyl)carbamate (200 mg, 640 μmol, 1.0 eq) in DMSO (12 mL) were added (5-formylthiophen-2-yl) boronic acid (100 mg, 640 μmol, 1.0 eq), KOAc (189 mg, 1.92 mmol, 3.0 eq), Pd(OAc) 2 (14.4 mg, 64.1 μmol, 0.1 eq), di-(1-adamantyl)-n-butylphosphine (45.9 mg, 128 μmol, 0.2 eq) and di-tert-butyl dicarbonate (140 mg, 640 μmol, 147 μL, 1.0 eq). The mixture was degassed and purged with N₂ three times, then stirred at 90° C. for 16 h under a N₂ atmosphere. TLC indicated that the starting material was completely consumed. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by preparative TLC (EtOAc/petroleum ether=1/3, R_{f}=0.5). tert-Butyl(1-(3-(5-formylthiophen-2-yl)phenyl)cyclopropyl)carbamate (150 mg, 437 μmol, 68% yield) was obtained as a yellow oil. M+H⁺=344.1 (LCMS).

### Step 2: 5-(3-(1-Aminocyclopropyl)phenyl)thiophene-2-carbaldehyde (202A-2)

To a solution of tert-butyl(1-(3-(5-formylthiophen-2-yl)phenyl)cyclopropyl)carbamate (120 mg, 349 μmol, 1.0 eq) in EtOAc (6.0 mL) was added HCl/EtOAc (4 M, 18 mL). The mixture was stirred at 20° C. for 1 h. LCMS indicated that the starting material was completely consumed, and the desired mass was detected. The reaction mixture was concentrated under vacuum to give the crude product 5-(3-(1-aminocyclopropyl)phenyl)thiophene-2-carbaldehyde (120 mg, HCl salt), which was used in the next step without any further purification. M+H⁺=244.1 (LCMS).

### Step 3: tert-Butyl 3-((3-((1-(3-(5-formylthiophen-2-yl)phenyl)cyclopropyl)carbamoyl)-4-methylphenyl)amino)azetidine-1-carboxylate (202A-3)

To a solution of 5-(3-(1-aminocyclopropyl)phenyl)thiophene-2-carbaldehyde (120 mg, 429 μmol, 1.0 eq, HCl salt) and 5-((1-(tert-butoxycarbonyl)azetidin-3-yl)amino)-2-methylbenzoic acid (131 mg, 429 μmol, 1.0 eq) in DCM (15 mL) were added TEA (130 mg, 1.29 mmol, 179 μL, 3.0 eq), EDCI (123 mg, 643 μmol, 1.5 eq) and HOBt (87.0 mg, 643 μmol, 1.5 eq). The mixture was stirred at 20° C. for 16 h. TLC indicated that the starting material was completely consumed. The reaction mixture was poured into H₂O (15 mL) and extracted with EtOAc (5.0 mL×3). The combined organic layers were dried over Na₂SO₄, filtered, and concentrated under vacuum to give a residue which was purified by preparative TLC (EtOAc/petroleum ether=1/2, R_{f}=0.2). tert-Butyl 3-((3-((1-(3-(5-formylthiophen-2-yl)phenyl)cyclopropyl)carbamoyl)-4-methylphenyl)amino)azetidine-1-carboxylate (250 mg, 409 μmol, 95% yield) was obtained as a yellow oil. M+H⁺=532.3 (LCMS).

### Step 4: tert-Butyl 3-((4-methyl-3-((1-(3-(5-(pyrrolidin-1-ylmethyl)thiophen-2-yl)phenyl)cyclopropyl)carbamoyl)phenyl)amino)azetidine-1-carboxylate (202A-4)

To a solution of pyrrolidine (8.03 mg, 113 μmol, 9.42 μL, 2.0 eq) and tert-butyl 3-((3-((1-(3-(5-formylthiophen-2-yl)phenyl)cyclopropyl)carbamoyl)-4-methylphenyl)amino)azetidine-1-carboxylate (30.0 mg, 56.3 μmol, 1.0 eq) in MeOH (3.0 mL) was added NaBH₃CN (7.09 mg, 113 μmol, 2.0 eq). The mixture was stirred at 20° C. for 16 h. LCMS indicated that the starting material was completely consumed, and the desired mass was detected. The reaction mixture was poured into H₂O (3.0 mL) and extracted with EtOAc (1.0 mL×3). The combined organic layers were dried over Na₂SO₄, filtered, and concentrated under vacuum to give the crude product tert-butyl 3-((4-methyl-3-((1-(3-(5-(pyrrolidin-1-ylmethyl)thiophen-2-yl)phenyl)cyclopropyl)carbamoyl)phenyl)amino)azetidine-1-carboxylate (40.0 mg), which was used in the next step without any further purification. M+H⁺=587.4 (LCMS).

### Step 5: 5-(Azetidin-3-ylamino)-2-methyl-N-(1-(3-(5-(pyrrolidin-1-ylmethyl)thiophen-2-yl)phenyl)cyclopropyl)benzamide (Compound 186)

To a solution of tert-butyl 3-((4-methyl-3-((1-(3-(5-(pyrrolidin-1-ylmethyl)thiophen-2-yl)phenyl)cyclopropyl)carbamoyl)phenyl)amino)azetidine-1-carboxylate (80.0 mg, 136 μmol, 1.0 eq) in EtOAc (5.0 mL) was added HCl/EtOAc (4 M, 5.0 mL). The mixture was stirred at 20° C. for 1 h. LCMS indicated that the starting material was completely consumed, and the desired mass was detected. The reaction mixture was concentrated under vacuum to give a residue which was purified by preparative HPLC (Phenomenex Luna column (80×30 mm, 3 μm); flow rate: 25 mL/min; gradient: 1%-30% B over 8 min; mobile phase A: 0.04% aqueous HCl, mobile phase B: acetonitrile). 5-(Azetidin-3-ylamino)-2-methyl-N-(1-(3-(5-(pyrrolidin-1-ylmethyl)thiophen-2-yl)phenyl)cyclopropyl)benzamide (30.0 mg, 57.3 μmol, 42% yield, HCl salt) was obtained as a white solid. M+H⁺=487.3 (LCMS); ¹H NMR (400 MHZ, DMSO-d₆) δ 11.21-10.87 (m, 1H), 9.31-9.08 (m, 2H), 9.04-8.87 (m, 1H), 7.53-7.45 (m, 3H), 7.42-7.34 (m, 2H), 7.17-7.11 (m, 1H), 7.03-6.95 (m, 1H), 6.60-6.55 (m, 1H), 6.53-6.48 (m, 1H), 4.76-4.49 (m, 2H), 4.46-4.32 (m, 1H), 4.28-4.16 (m, 2H), 3.87-3.83 (m, 2H), 3.46-3.37 (m, 2H), 3.18-3.01 (m, 2H), 2.21-2.15 (m, 3H), 2.05-1.82 (m, 4H), 1.35-1.22 (m, 4H).

### Example 203: 5-(Azetidin-3-ylamino)-N-(1-(3-(5-((((1S,3R)-3-hydroxycyclopentyl)amino)methyl)thiophen-2-yl)phenyl)cyclopropyl)-2-methylbenzamide (Compound 196)

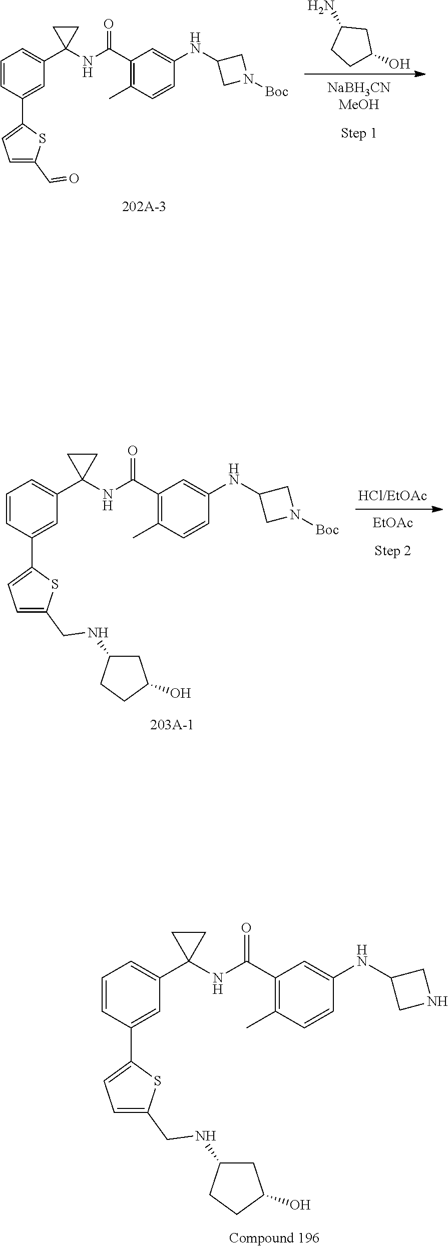

### Step 1: tert-Butyl 3-((3-((1-(3-(5-((((1S,3R)-3-hydroxycyclopentyl)amino)methyl)thiophen-2-yl)phenyl)cyclopropyl)carbamoyl)-4-methylphenyl)amino)azetidine-1-carboxylate (203A-1)

To a solution of tert-butyl 3-((3-((1-(3-(5-formylthiophen-2-yl)phenyl)cyclopropyl) carbamoyl)-4-methylphenyl)amino)azetidine-1-carboxylate (130 mg, 245 μmol, 1.0 eq) and (1R,3S)-3-aminocyclopentanol (49.6 mg, 489 μmol, 2.0 eq) in MeOH (6.0 mL) was added NaBH₃CN (30.7 mg, 489 μmol, 2.0 eq). The mixture was stirred at 20° C. for 20 h. LCMS indicated that the starting material was completely consumed, and the desired mass was detected. The reaction mixture was poured into H₂O (6.0 mL) and extracted with EtOAc (2.0 mL×3). The combined organic layers were dried over Na₂SO₄, filtered, and concentrated under vacuum to give a residue which was purified by preparative HPLC (Phenomenex C18 column (75×30 mm, 3 μm); flow rate: 25 mL/min; gradient: 30%-60% B over 8 min; mobile phase A: 10 mM aqueous NH₄HCO₃, mobile phase B: acetonitrile). tert-Butyl 3-((3-((1-(3-(5-((((1S,3R)-3-hydroxycyclopentyl)amino)methyl)thiophen-2-yl)phenyl)cyclopropyl)carbamoyl)-4-methylphenyl)amino)azetidine-1-carboxylate (40.0 mg, 59.0 μmol, 24% yield) was obtained as a colorless oil. M+H⁺=617.4 (LCMS).

### Step 2: 5-(Azetidin-3-ylamino)-N-(1-(3-(5-((((1S,3R)-3-hydroxycyclopentyl)amino)methyl)thiophen-2-yl)phenyl)cyclopropyl)-2-methylbenzamide (Compound 196)

To a solution of tert-butyl 3-((3-((1-(3-(5-((((1S,3R)-3-hydroxycyclopentyl)amino)methyl)thiophen-2-yl)phenyl)cyclopropyl)carbamoyl)-4-methylphenyl)amino)azetidine-1-carboxylate (30.0 mg, 48.6 μmol, 1.0 eq) in EtOAc (3.0 mL) was added HCl/EtOAc (4 M, 2.5 mL). The mixture was stirred at 20° C. for 20 min. LCMS indicated that the starting material was completely consumed, and the desired mass was detected. The reaction mixture was concentrated under vacuum to give a residue which was purified by preparative HPLC (Phenomenex Luna column (80×30 mm, 3 μm); flow rate: 25 mL/min; gradient: 1%-25% B over 8 min; mobile phase A: 0.04% aqueous HCl, mobile phase B: acetonitrile). 5-(Azetidin-3-ylamino)-N-(1-(3-(5-((((1S,3R)-3-hydroxycyclopentyl)amino)methyl)thiophen-2-yl)phenyl)cyclopropyl)-2-methyl benzamide (24.4 mg, 44.1 μmol, 91% yield, HCl salt) was obtained as a white solid. M+H⁺=517.2 (LCMS); ¹H NMR (400 MHZ, DMSO-d₆) δ 9.31 (br d, J=4.1 Hz, 2H), 9.25-9.06 (m, 2H), 8.98-8.92 (m, 1H), 7.54-7.42 (m, 3H), 7.41-7.32 (m, 2H), 7.17-7.10 (m, 1H), 6.99 (d, J=8.4 Hz, 1H), 6.60-6.48 (m, 2H), 4.42-4.32 (m, 3H), 4.23 (br d, J=7.3 Hz, 2H), 4.14-4.02 (m, 2H), 3.89-3.71 (m, 2H), 2.25-2.13 (m, 4H), 1.99-1.84 (m, 2H), 1.76-1.60 (m, 3H), 1.35-1.22 (m, 4H).

### Example 204: 5-(2-(Dimethylamino)ethoxy)-N-(1-(2,3-dimethylphenyl)cyclopropyl)-2-methylbenzamide (Compound 318)

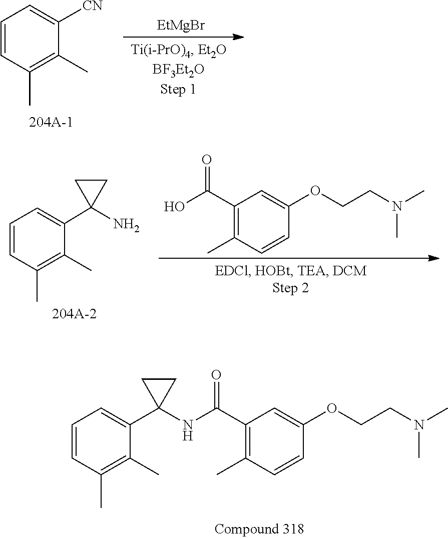

### Step 1: 1-(2,3-Dimethylphenyl)cyclopropanamine (204A-2)

A mixture of 2,3-dimethylbenzonitrile (1.00 g, 7.62 mmol, 1.0 eq) in anhydrous Et₂O (50 mL) was degassed and purged with N₂ three times. To this mixture was added Ti(i-PrO)+ (2.17 g, 7.62 mmol, 2.25 mL, 1.0 eq) slowly at −78° C. and then EtMgBr (3 M in Et₂O, 5.59 mL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. BF₃·Et₂O (2.16 g, 15.3 mmol, 1.88 mL, 2.0 eq) was added slowly with no obvious temperature change. The mixture was stirred at 20° C. for another 1 h. TLC indicated that the starting material was completely consumed, and a main spot was detected. The reaction mixture was added into a mixture of HCl (1 M aqueous) (30 mL) and MTBE (30 mL) and extracted with MTBE (30 mL×4). The aqueous layer was basified to pH 8 using NaOH (2 M aqueous) and a precipitate was formed. The mixture was filtered through a pad of Celite and the slurry was washed with DCM several times. The combined filtrate was extracted with DCM (50 mL×4). The combined organic layers were dried over Na₂SO₄, filtered, and concentrated under vacuum to give a residue which was purified by flash silica gel chromatography using a gradient of EtOAc/petroleum ether from 0/1 to 1/1. 1-(2,3-Dimethylphenyl)cyclopropanamine (100 mg, 620 μmol, 8% yield) was obtained as a yellow oil. M+H⁺=162.2 (LCMS).

### Step 2: 5-(2-(Dimethylamino)ethoxy)-N-(1-(2,3-dimethylphenyl)cyclopropyl)-2-methylbenzamide (Compound 318)

To a solution of 5-(2-(dimethylamino)ethoxy)-2-methylbenzoic acid (166 mg, 744 μmol, 1.2 eq) in DCM (3.0 mL) were added TEA (188 mg, 1.86 mmol, 259 μL, 3.0 eq), EDCI (178 mg, 930 μmol, 1.5 eq), HOBt (126 mg, 930 μmol, 1.5 eq) and 1-(2,3-dimethylphenyl)cyclopropanamine (100 mg, 620 μmol, 1.0 eq). The mixture was stirred at 20° C. for 16 h. LCMS indicated that the starting material was completely consumed, and the desired mass was detected. The reaction mixture was poured into H₂O (5.0 mL) and extracted with EtOAc (5.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 15%-40% B over 8 min; mobile phase A: 0.04% aqueous HCl, mobile phase B: acetonitrile). 5-(2-(Dimethylamino)ethoxy)-N-(1-(2,3-dimethylphenyl)cyclopropyl)-2-methylbenzamide (51.0 mg, 139 μmol, 22% yield, HCl salt) was obtained as a white solid. M+H⁺=367.2 (LCMS); ¹H NMR (400 MHZ, DMSO-d₆) δ 10.56 (br s, 1H), 8.85 (s, 1H), 7.49-7.44 (m, 1H), 7.11 (d, J=8.5 Hz, 1H), 7.05-6.97 (m, 2H), 6.94-6.90 (m, 1H), 6.76 (d, J=2.6 Hz, 1H), 4.31 (t, J=5.1 Hz, 2H), 3.49-3.40 (m, 2H), 2.80 (d, J=4.9 Hz, 6H), 2.39 (s, 3H), 2.23 (s, 3H), 2.09 (s, 3H), 1.22-1.16 (m, 2H), 1.05-0.98 (m, 2H).

### Example 205: N-(1-(2,3-Dichlorophenyl)cyclopropyl)-5-(2-(dimethylamino)ethoxy)-2-methylbenzamide (Compound 335)

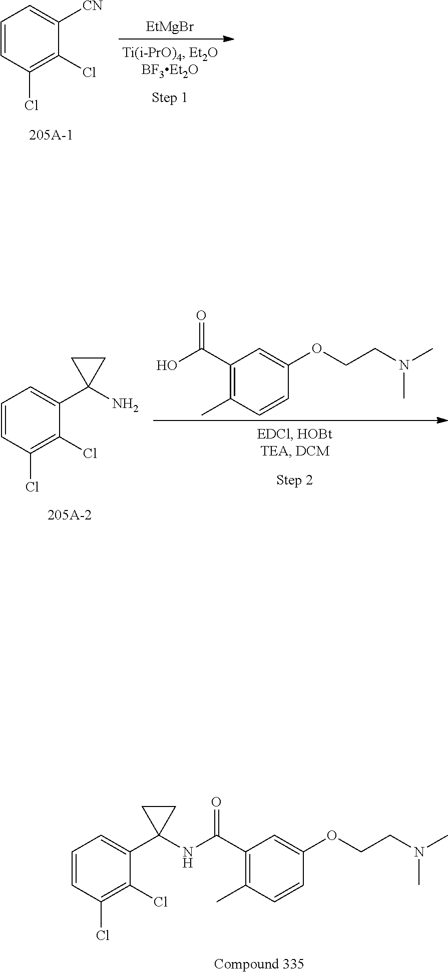 [SMILES: "EtMgBr" ; N#Cc1cccc(Cl)c1Cl ; NC1(c2cccc(Cl)c2Cl)CC1 ; Cc1ccc(OCCN(C)C)cc1C(=O)O ; Cc1ccc(OCCN(C)C)cc1C(=O)NC1(c2cccc(Cl)c2Cl)CC1]

### Step 1: 1-(2,3-Dichlorophenyl)cyclopropanamine (205A-2)

A mixture of 2,3-dichlorobenzonitrile (200 mg, 1.16 mmol, 1.0 eq) in anhydrous Et₂O (15 mL) was degassed and purged with N₂ three times. The mixture was stirred at −78° C. To this mixture was added Ti(i-PrO)⁴ (363 mg, 1.28 mmol, 377 μL, 1.1 eq) slowly, and then EtMgBr (3 M in Et₂O, 853 μL, 2.2 eq) was added dropwise to maintain the temperature between −78° C. and −75° C. over 1 h under a N₂ atmosphere. After the addition was complete, the resulting mixture was stirred at the same temperature for 10 min and warmed to 20° C. over 1 h. BF₃·Et₂O (330 mg, 2.33 mmol, 287 μmL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (2.0 mL) and MTBE (15 mL), and extracted with MTBE (15 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (20 mL×2). The combined organic layers were dried over Na₂SO₄, filtered, and concentrated under vacuum to give a residue which was purified by preparative TLC (EtOAc/petroleum ether=1/1, R_{f}=0.2). 1-(2,3-Dichlorophenyl)cyclopropanamine (70.0 mg, 346 μmol, 30% yield) was obtained as a yellow oil. M+H⁺=202.0 (LCMS).

### Step 2: N-(1-(2,3-Dichlorophenyl)cyclopropyl)-5-(2-(dimethylamino)ethoxy)-2-methylbenzamide (Compound 335)

To a solution of 1-(2,3-dichlorophenyl)cyclopropanamine (50.0 mg, 247 μmol, 1.0 eq) and 5-[2-(dimethylamino)ethoxy]-2-methyl-benzoic acid (55.2 mg, 247 μmol, 1.0 eq) in DMF (1.0 mL) were added HATU (141 mg, 371 μmol, 1.5 eq) and DIEA (95.9 mg, 742 μmol, 100 μL, 3.0 eq). The mixture was stirred at 25° C. for 1 h. LCMS indicated that the starting material was completely consumed, and the desired mass was detected. The reaction mixture was filtered and the filtrate was purified by preparative HPLC (Phenomenex Luna column (80×30 mm, 3 μm); flow rate: 25 mL/min; gradient: 20%-40% B over 8 min; mobile phase A: 0.04% aqueous HCl, mobile phase B: acetonitrile) to give N-(1-(2,3-dichlorophenyl)cyclopropyl)-5-(2-(dimethylamino)ethoxy)-2-methylbenzamide (34.4 mg, 71.5 μmol, 29% yield, HCl salt) as a white solid. M+H⁺=407.0 (LCMS); ¹H NMR (400 MHZ, DMSO-d₆) δ 10.09 (br s, 1H), 9.02-8.92 (m, 1H), 7.70 (dd, J=1.2, 7.8 Hz, 1H), 7.54 (dd, J=1.2, 8.0 Hz, 1H), 7.33 (t, J=8.0 Hz, 1H), 7.13 (d, J=8.4 Hz, 1H), 6.97-6.91 (m, 1H), 6.80 (d, J=2.8 Hz, 1H), 4.29 (t, J=4.8 Hz, 2H), 3.47 (q, J=4.8 Hz, 2H), 2.82 (d, J=4.8 Hz, 6H), 2.10 (s, 3H), 1.26-1.19 (m, 2H), 1.19-1.13 (m, 2H).

### Example 206: N-(1-(2-Bromo-3-methylphenyl)cyclopropyl)-5-(2-(dimethylamino)ethoxy)-2-methylbenzamide (Compound 317)

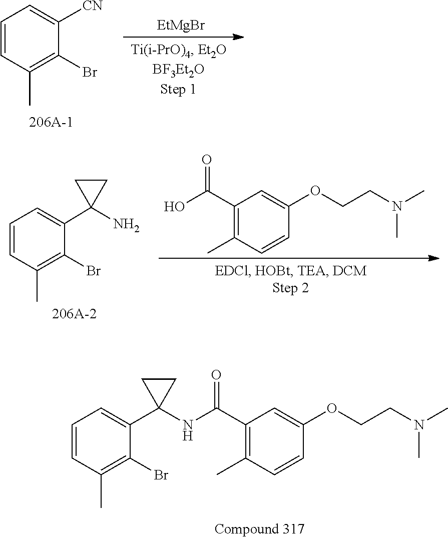

### Step 1: 1-(2-Bromo-3-methylphenyl)cyclopropanamine (206A-2)

A mixture of 2-bromo-3-methylbenzonitrile (200 mg, 1.02 mmol, 1.0 eq) in anhydrous Et₂O (15 mL) was degassed and purged with N₂ three times. To this mixture was added Ti(i-PrO)₄ (290 mg, 1.02 mmol, 301 μL, 1.0 eq) slowly at −78° C. and then EtMgBr (3 M in Et₂O, 748 μL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. BF₃·Et₂O (290 mg, 2.04 mmol, 252 μL, 2.0 eq) was added slowly with no obvious temperature change. The mixture was stirred at 20° C. for another 1 h. TLC indicated that the starting material was completely consumed. The reaction mixture was added into a mixture of HCl (1 M aqueous) (10 mL) and MTBE (10 mL) and extracted with MTBE (10 mL×4). The aqueous layer was basified to pH 8 using NaOH (2 M aqueous) and a precipitate was formed. The mixture was filtered through a pad of Celite and the slurry was washed with DCM several times. The combined filtrate was extracted with DCM (10 mL×4). The combined organic layers were dried over Na₂SO₄, filtered, and concentrated under vacuum to give a residue which was purified by flash silica gel chromatography using a gradient of EtOAc/petroleum ether from 0/1 to 1/1. 1-(2-Bromo-3-methylphenyl)cyclopropanamine (150 mg, 633 μmol, 65% yield) was obtained as a yellow oil. M+H⁺=226.2 (LCMS).

### Step 2: N-(1-(2-Bromo-3-methylphenyl)cyclopropyl)-5-(2-(dimethylamino)ethoxy)-2-methylbenzamide (Compound 317)

To a solution of 5-(2-(dimethylamino)ethoxy)-2-methylbenzoic acid (82.9 mg, 372 μmol, 1.2 eq) in DCM (2.0 mL) were added TEA (94.0 mg, 929 μmol, 129 μL, 3.0 eq), EDCI (89.0 mg, 464 μmol, 1.5 eq), HOBt (62.8 mg, 464 μmol, 1.5 eq) and 1-(2-bromo-3-methylphenyl)cyclopropanamine (70.0 mg, 310 μmol, 1.0 eq). The mixture was stirred at 20° C. for 16 h. LCMS indicated that the starting material was completely consumed, and the desired mass was detected. The reaction mixture was poured into H₂O (5.0 mL) and extracted with EtOAc (5.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 20%-45% B over 8 min; mobile phase A: 0.04% aqueous HCl, mobile phase B: acetonitrile). N-(1-(2-Bromo-3-methylphenyl)cyclopropyl)-5-(2-(dimethylamino)ethoxy)-2-methylbenzamide (35.7 mg, 82.8 μmol, 27% yield, HCl salt) was obtained as a yellow solid. M+H⁺=431.1 (LCMS); ¹H NMR (400 MHZ, DMSO-d₆) δ 10.75 (br s, 1H), 8.79 (s, 1H), 7.59-7.52 (m, 1H), 7.27-7.18 (m, 2H), 7.11 (d, J=8.5 Hz, 1H), 6.96-6.90 (m, 1H), 6.81 (d, J=2.6 Hz, 1H), 4.32 (br d, J=5.1 Hz, 2H), 3.53-3.40 (m, 2H), 2.80 (d, J=4.9 Hz, 6H), 2.37 (s, 3H), 2.11 (s, 3H), 1.29-1.20 (m, 2H), 1.14-1.05 (m, 2H).

### Example 207: N-(1-(3-Bromo-2-methylphenyl)cyclopropyl)-5-(2-(dimethylamino)ethoxy)-2-methylbenzamide (Compound 336)

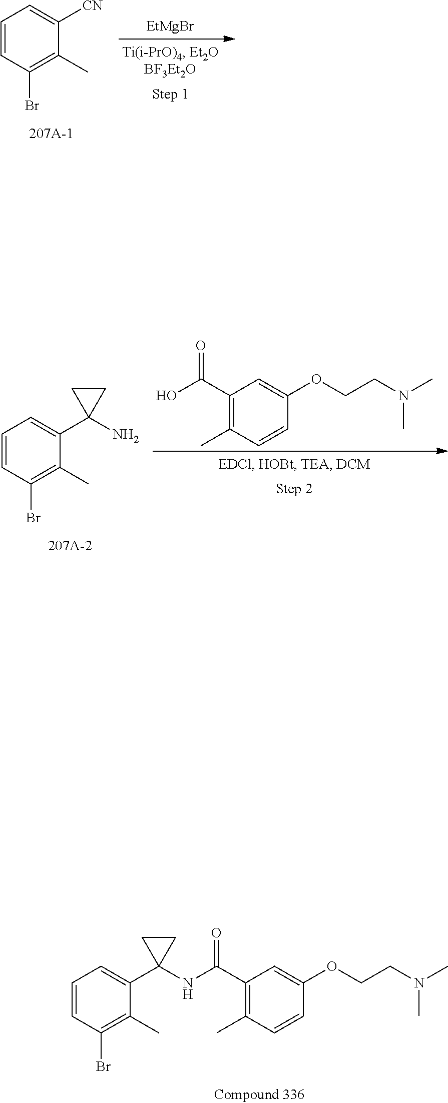

### Step 1: 1-(3-Bromo-2-methylphenyl)cyclopropanamine (207A-2)

A mixture of 3-bromo-2-methylbenzonitrile (500 mg, 2.55 mmol, 1.0 eq) in anhydrous Et₂O (25 mL) was degassed and purged with N₂ three times. The mixture was stirred at −78° C. To this mixture was added Ti(i-PrO)₄ (725 mg, 2.55 mmol, 753 μL, 1.0 eq) slowly and then EtMgBr (3 M in Et₂O, 1.87 mL, 2.2 eq) was added dropwise to maintain the temperature between −78° C. and −75° C. over 1 h under a N₂ atmosphere. After the addition was complete, the resulting mixture was stirred at the same temperature for 10 min and warmed to 20° C. over 1 h. BF₃·Et₂O (724 mg, 5.10 mmol, 630 μL, 2.0 eq) was added slowly with no obvious temperature change. The mixture was stirred at 20° C. for another 1 h. TLC indicated that that the starting material was completely consumed, and a main spot was detected. The reaction mixture was added into a mixture of HCl (1 M aqueous) (15 mL) and MTBE (15 mL) and extracted with MTBE (15 mL×4). The aqueous layer was basified to pH 8 using NaOH (2 M aqueous) and a precipitate was formed. The mixture was filtered through a pad of Celite and the slurry was washed with DCM several times. The combined filtrate was extracted with DCM (25 mL×4). The combined organic layers were dried over Na₂SO₄, filtered, and concentrated under vacuum to give a residue which was purified by flash silica gel chromatography using a gradient of EtOAc/petroleum ether from 0/1 to 1/1. 1-(3-Bromo-2-methylphenyl)cyclopropanamine (140 mg, 619 μmol, 24% yield) was obtained as a yellow gum. M+H⁺=226.0 (LCMS); ¹H NMR (400 MHZ, DMSO-d₆) δ 7.43 (d, J=7.9 Hz, 1H), 7.27 (d, J=7.5 Hz, 1H), 7.03 (t, J=7.8 Hz, 1H), 2.48 (br d, J=1.6 Hz, 3H), 0.88-0.83 (m, 2H), 0.77-0.70 (m, 2H).

### Step 2: N-(1-(3-Bromo-2-methylphenyl)cyclopropyl)-5-(2-(dimethylamino)ethoxy)-2-methylbenzamide (Compound 336)

To a solution of 5-(2-(dimethylamino)ethoxy)-2-methylbenzoic acid (166 mg, 743 μmol, 1.2 eq) in DCM (3.0 mL) were added TEA (188 mg, 1.86 mmol, 259 μL, 3.0 eq), EDCI (178 mg, 939 μmol, 1.5 eq), HOBt (125 mg, 939 μmol, 1.5 eq) and 1-(3-bromo-2-methylphenyl)cyclopropanamine (140 mg, 619 μmol, 1.0 eq). The mixture was stirred at 25° C. for 16 h. LCMS indicated that the starting material was completely consumed, and the desired mass was detected. The reaction mixture was poured into H₂O (5.0 mL) at 25° C. and extracted with EtOAc (5.0 mL×3). The combined organic layers were washed with brine (5.0 mL×3), dried over Na₂SO₄, filtered, and concentrated under vacuum to give a residue which was purified by preparative HPLC (Phenomenex Luna column (80×30 mm, 3 μm); flow rate: 25 mL/min; gradient: 15%-40% B over 8 min; mobile phase A: 0.04% aqueous HCl, mobile phase B: acetonitrile). N-(1-(3-Bromo-2-methylphenyl)cyclopropyl)-5-(2-(dimethylamino)ethoxy)-2-methylbenzamide (130 mg, 301 μmol, 49% yield, HCl salt) was obtained as a yellow solid. M+H⁺=433.1 (LCMS); ¹H NMR (400 MHZ, DMSO-d₆) δ 9.97-9.83 (m, 1H), 8.95 (s, 1H), 7.66 (d, J=7.8 Hz, 1H), 7.50 (d, J=7.9 Hz, 1H), 7.15-7.06 (m, 2H), 6.96-6.91 (m, 1H), 6.78 (d, J=2.6 Hz, 1H), 4.29 (t, J=5.0 Hz, 2H), 3.49-3.45 (m, 2H), 2.83 (d, J=4.9 Hz, 6H), 2.56 (s, 3H), 2.10 (s, 3H), 1.23-1.18 (m, 2H), 1.14-1.07 (m, 2H).

### Example 208: 5-(2-(Dimethylamino)ethoxy)-2-methyl-N-(1-(2-methyl-[1,1′-biphenyl]-3-yl)cyclopropyl)benzamide (Compound 340)

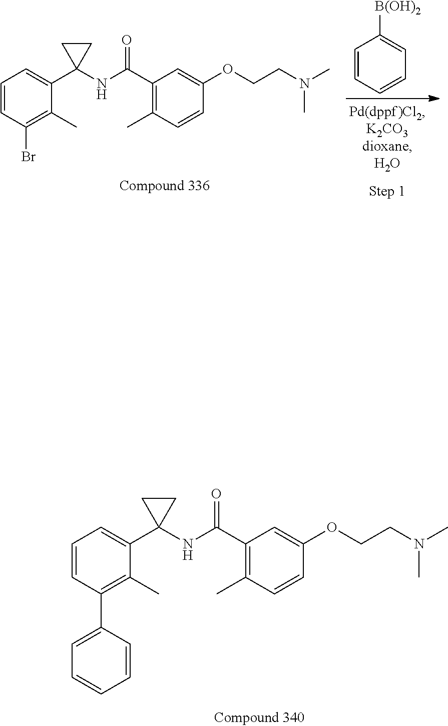

### Step 1: 5-(2-(Dimethylamino)ethoxy)-2-methyl-N-(1-(2-methyl-[1,1′-biphenyl]-3-yl)cyclopropyl)benzamide (Compound 340)

To a solution of N-(1-(3-bromo-2-methylphenyl)cyclopropyl)-5-(2-(dimethylamino)ethoxy)-2-methylbenzamide (80.0 mg, 185 μmol, 1.0 eq) in a mixture of dioxane (3.0 mL) and H₂O (0.8 mL) were added phenylboronic acid (33.9 mg, 278 μmol, 1.5 eq), K₂CO₃ (76.9 mg, 556 μmol, 3.0 eq) and Pd(dppf)Cl₂ (13.6 mg, 18.6 μmol, 0.1 eq) at 25° C. The mixture was heated to 90° C. and stirred for 16 h under a N₂ atmosphere. LCMS indicated that the starting material was completely consumed, and the desired mass was detected. The reaction mixture was allowed to cool to room temperature, poured into H₂O (3.0 mL) at 25° C. and extracted with EtOAc (3.0 mL×3). The combined organic layers were washed with brine (3.0 mL×3), dried over Na₂SO₄, filtered, and concentrated under vacuum to give a residue which was purified by preparative HPLC (Phenomenex Luna column (80×30 mm, 3 μm); flow rate: 25 mL/min; gradient: 20%-50% B over 8 min; mobile phase A: 0.04% aqueous HCl, mobile phase B: acetonitrile). 5-(2-(Dimethylamino)ethoxy)-2-methyl-N-(1-(2-methyl-[1,1′-biphenyl]-3-yl)cyclopropyl)benzamide (57.8 mg, 135 μmol, 73% yield, HCl salt) was obtained as a white solid. M+H⁺=429.2 (LCMS); ¹H NMR (400 MHZ, DMSO-d₆) δ 10.07 (br s, 1H), 8.89 (s, 1H), 7.69-7.64 (m, 1H), 7.47-7.42 (m, 2H), 7.38-7.34 (m, 1H), 7.32-7.28 (m, 2H), 7.21-7.17 (m, 1H), 7.13 (d, J=8.4 Hz, 1H), 7.09-7.06 (m, 1H), 6.95-6.91 (m, 1H), 6.80 (d, J=2.6 Hz, 1H), 4.29 (t, J=5.0 Hz, 2H), 3.48-3.44 (m, 2H), 2.82 (d, J=4.5 Hz, 6H), 2.37 (s, 3H), 2.12 (s, 3H), 1.24-1.19 (m, 2H), 1.15-1.10 (m, 2H).

### Example 209: 5-(2-(Dimethylamino)ethoxy)-2-methyl-N-(1-(5,6,7,8-tetrahydronaphthalen-1-yl)cyclopropyl)benzamide (Compound 329)

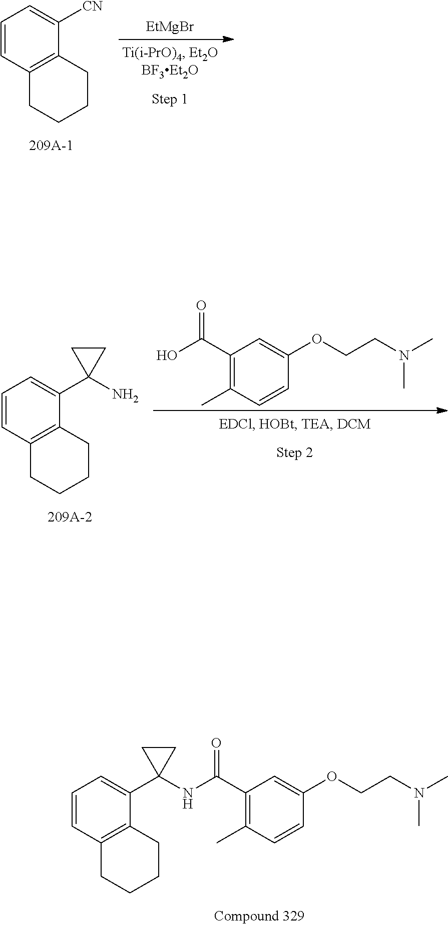 [SMILES: "EtMgBr" ; N#Cc1cccc2c1CCCC2 ; NC1(c2cccc3c2CCCC3)CC1 ; Cc1ccc(OCCN(C)C)cc1C(=O)O ; Cc1ccc(OCCN(C)C)cc1C(=O)NC1(c2cccc3c2CCCC3)CC1]

### Step 1: 1-(5,6,7,8-Tetrahydronaphthalen-1-yl)cyclopropanamine (209A-2)

A mixture of 5,6,7,8-tetrahydronaphthalene-1-carbonitrile (200 mg, 1.27 mmol, 1.0 eq) in anhydrous Et₂O (15 mL) was degassed and purged with N₂ three times. The mixture was stirred at −78° C. To this mixture was added Ti(i-PrO)₄ (361 mg, 1.27 mmol, 375 μL, 1.0 eq) slowly and then EtMgBr (3 M in Et₂O, 933 μL, 2.2 eq) was added dropwise to maintain the temperature between −78° C. and −75° C. over 1 h under a N₂ atmosphere. After the addition was complete, the resulting mixture was stirred at the same temperature for 10 min and warmed to 20° C. over 1 h. BF₃·Et₂O (361 mg, 2.54 mmol, 314 μL, 2.0 eq) was added slowly with no obvious temperature change. The mixture was stirred at 20° C. for another 1 h. TLC indicated that the starting material was completely consumed, and a main spot was detected. The reaction mixture was added into a mixture of HCl (1 M aqueous) (10 mL) and MTBE (10 mL) and extracted with MTBE (10 mL×4). The aqueous layer was basified to pH 8 using NaOH (2 M aqueous) and a precipitate was formed. The mixture was filtered through a pad of Celite and the slurry was washed with DCM several times. The combined filtrate was extracted with DCM (20 mL×4). The combined organic layers were dried over Na₂SO₄, filtered, and concentrated under vacuum to give a residue which was purified by flash silica gel chromatography using a gradient of EtOAc/petroleum ether from 0/1 to 1/1. 1-(5,6,7,8-Tetrahydronaphthalen-1-yl)cyclopropanamine (60.0 mg, 320 μmol, 25% yield) was obtained as a yellow gum. M+H⁺=188.1 (LCMS); ¹H NMR (400 MHZ, DMSO-d₆) δ 7.08-7.04 (m, 1H), 6.98 (t, J=7.5 Hz, 1H), 6.92-6.87 (m, 1H), 2.94 (t, J=6.0 Hz, 2H), 2.73-2.69 (m, 2H), 1.77-1.67 (m, 4H), 0.84-0.76 (m, 2H), 0.72-0.65 (m, 2H).

### Step 2: 5-(2-(Dimethylamino)ethoxy)-2-methyl-N-(1-(5,6,7,8-tetrahydronaphthalen-1-yl)cyclopropyl)benzamide (Compound 329)

To a solution of 5-(2-(dimethylamino)ethoxy)-2-methylbenzoic acid (85.8 mg, 384 μmol, 1.2 eq) in DCM (3.0 mL) were added TEA (97.3 mg, 961 μmol, 134 μL, 3.0 eq), EDCI (92.1 mg, 481 μmol, 1.5 eq), HOBt (64.9 mg, 481 μmol, 1.5 eq) and 1-(5,6,7,8-tetrahydronaphthalen-1-yl)cyclopropanamine (60.0 mg, 320 μmol, 1.0 eq). The mixture was stirred at 25° C. for 16 h. LCMS indicated that the starting material was completely consumed, and the desired mass was detected. The reaction mixture was poured into H₂O (5.0 mL) at 25° C. and extracted with EtOAc (5.0 mL×3). The combined organic layers were washed with brine (5.0 mL×3), dried over Na₂SO₄, filtered, and concentrated under vacuum to give a residue which was purified by preparative HPLC (Phenomenex Luna column (80×30 mm, 3 μm); flow rate: 25 mL/min; gradient: 20%-45% B over 8 min; mobile phase A: 0.04% aqueous HCl, mobile phase B: acetonitrile). 5-(2-(Dimethylamino)ethoxy)-2-methyl-N-(1-(5,6,7,8-tetrahydronaphthalen-1-yl)cyclopropyl)benzamide (15.0 mg, 38.2 μmol, 12% yield, HCl salt) was obtained as a white solid. M+H⁺=393.2 (LCMS); ¹H NMR (400 MHZ, DMSO-d₆) δ 10.48-10.27 (m, 1H), 8.78 (s, 1H), 7.41 (br d, J=7.4 Hz, 1H), 7.11 (br d, J=8.4 Hz, 1H), 7.05-6.96 (m, 1H), 6.96-6.89 (m, 2H), 6.76 (s, 1H), 4.30 (br s, 2H), 3.46 (br d, J=4.3 Hz, 2H), 2.96 (br s, 2H), 2.81 (br d, J=3.9 Hz, 6H), 2.73 (br s, 2H), 2.09 (s, 3H), 1.74 (br d, J=4.4 Hz, 4H), 1.14 (br s, 2H), 1.02 (br s, 2H).

### Example 210: N-(1-(Benzofuran-7-yl)cyclopropyl)-5-(2-(dimethylamino)ethoxy)-2-methylbenzamide (Compound 370)

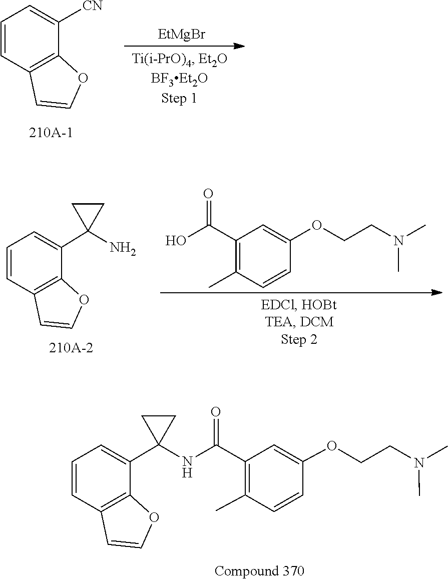 [SMILES: "EtMgBr" ; N#Cc1cccc2ccoc12 ; NC1(c2cccc3ccoc23)CC1 ; Cc1ccc(OCCN(C)C)cc1C(=O)O ; Cc1ccc(OCCN(C)C)cc1C(=O)NC1(c2cccc3ccoc23)CC1]

### Step 1: 1-(Benzofuran-7-yl)cyclopropanamine (210A-2)

To a mixture of benzofuran-7-carbonitrile (200 mg, 1.40 mmol, 1.0 eq) in anhydrous Et₂O (20 mL) was degassed and purged with N₂ three times. The mixture was stirred at −78° C. To this mixture was added Ti(i-PrO)⁴ (437 mg, 1.54 mmol, 450 μL, 1.1 eq) slowly, and then EtMgBr (3 M in Et₂O, 1.02 mL, 2.2 eq) was added dropwise to maintain the temperature between-78° C. and −75° C. over 5 min under a N₂ atmosphere. After the addition was complete, the resulting mixture was stirred at the same temperature for 10 min and warmed to 20° C. over 1 h. BF₃·Et₂O (397 mg, 2.79 mmol, 350 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (20 mL) and MTBE (20 mL), and extracted with MTBE (20 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (20 mL×2). The combined organic layers were dried over Na₂SO₄, filtered, and concentrated under vacuum to give a residue which was purified by preparative TLC (DCM/MeOH=10/1, R_{f}=0.2). 1-(Benzofuran-7-yl)cyclopropanamine (60.0 mg, 0.35 mmol, 25% yield) was obtained as a yellow oil. M+H⁺=174.1 (LCMS).

### Step 2: N-(1-(Benzofuran-7-yl)cyclopropyl)-5-(2-(dimethylamino)ethoxy)-2-methylbenzamide (Compound 370)

To a solution of 1-(benzofuran-7-yl)cyclopropanamine (50.0 mg, 289 μmol, 1.0 eq) and 5-(2-(dimethylamino)ethoxy)-2-methylbenzoic acid (64.5 mg, 289 μmol, 1.0 eq) in DCM (3.0 mL) were added TEA (58.4 mg, 0.58 mmol, 80.3 μL, 2.0 eq), EDCI (83.0 mg, 0.43 mmol, 1.5 eq) and HOBt (58.5 mg, 0.43 mmol, 1.5 eq). The mixture was stirred at 20° C. for 2 h. LCMS indicated that the starting material was completely consumed, and the desired mass was detected. The reaction mixture was poured into H₂O (3.0 mL) and extracted with EtOAc (3.0 mL×2). The combined organic layers were dried over Na₂SO₄, filtered, and concentrated under vacuum to give a residue which was purified by preparative HPLC (Phenomenex Gemini C18 column (100×40 mm, 3 μm); flow rate: 50 mL/min; gradient: 10%-50% B over 8 min; mobile phase A: 0.2% aqueous FA, mobile phase B: acetonitrile). N-(1-(Benzofuran-7-yl)cyclopropyl)-5-(2-(dimethylamino)ethoxy)-2-methylbenzamide (50.0 mg, 118 μmol, 41% yield, FA salt) was obtained as a white gum. M+H⁺=379.1 (LCMS); ¹H NMR (400 MHZ, DMSO-d₆) δ 9.08 (s, 1H), 8.18 (s, 1H), 7.88-8.04 (m, 1H), 7.45-7.55 (m, 1H), 7.29-7.37 (m, 1H), 7.17-7.24 (m, 1H), 7.08-7.14 (m, 1H), 6.87-6.97 (m, 3H), 4.07 (t, J=5.68 Hz, 2H), 2.62-2.79 (m, 2H), 2.27 (s, 6H), 2.16 (s, 3H), 1.50-1.67 (m, 2H), 1.27 (br d, J=1.71 Hz, 2H).

### Example 211: N-(1-(Benzofuran-4-yl)cyclopropyl)-5-(2-(dimethylamino)ethoxy)-2-methylbenzamide (Compound 357)

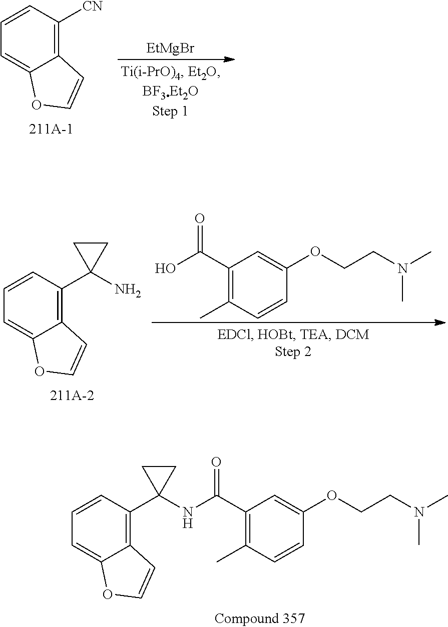 [SMILES: "EtMgBr" ; N#Cc1cccc2occc12 ; NC1(c2cccc3occc23)CC1 ; Cc1ccc(OCCN(C)C)cc1C(=O)O ; Cc1ccc(OCCN(C)C)cc1C(=O)NC1(c2cccc3occc23)CC1]

### Step 1: 1-(Benzofuran-4-yl)cyclopropanamine (211A-2)

A suspension of benzofuran-4-carbonitrile (200 mg, 1.40 mmol, 1.0 eq) in anhydrous Et₂O (15 mL) was degassed and purged with N₂ three times. To this mixture was added Ti(i-PrO)₄ (397 mg, 1.40 mmol, 412 μL, 1.0 eq) slowly at −78° C. and then EtMgBr (3 M in Et₂O, 1.02 mL, 2.2 eq) was added dropwise to maintain the temperature between −78° C. and −75° C. under a N₂ atmosphere. After the addition was complete, the resulting mixture was stirred at the same temperature for 10 min and warmed to 20° C. over 1 h. BF₃·Et₂O (397 mg, 2.79 mmol, 345 μL, 2.0 eq) was added slowly with no obvious temperature change. The mixture was stirred at 20° C. for another 1 h. LCMS indicated that the starting material was completely consumed, and the desired mass was detected. The reaction mixture was poured into a mixture of HCl (1 M aqueous) (10 mL) and MTBE (10 mL) and extracted with MTBE (10 mL×4). The aqueous layer was basified to pH 8 using NaOH (2 M aqueous) and a precipitate was formed. The mixture was filtered through a pad of Celite and the slurry was washed with DCM several times. The combined filtrate was extracted with DCM (15 mL×4). The combined organic layers were dried over Na₂SO₄, filtered, and concentrated under vacuum to give the crude product 1-(benzofuran-4-yl)cyclopropanamine (120 mg), which was used in the next step without any further purification. M+H⁺=174.1 (LCMS); ¹H NMR (400 MHZ, DMSO-d₆) δ 7.97 (d, J=2.2 Hz, 1H), 7.46-7.40 (m, 1H), 7.22-7.16 (m, 3H), 0.93 (t, J=2.3 Hz, 2H), 0.90 (t, J=2.3 Hz, 2H).

### Step 2: N-(1-(Benzofuran-4-yl)cyclopropyl)-5-(2-(dimethylamino)ethoxy)-2-methylbenzamide (Compound 357)

To a solution of 5-(2-(dimethylamino)ethoxy)-2-methylbenzoic acid (170 mg, 762 μmol, 1.2 eq) in DCM (3.0 mL) were added TEA (193 mg, 1.91 mmol, 265 μL, 3.0 eq), EDCI (183 mg, 953 μmol, 1.5 eq), HOBt (129 mg, 953 μmol, 1.5 eq) and 1-(benzofuran-4-yl)cyclopropanamine (110 mg, 635 μmol, 1.0 eq). The mixture was stirred at 25° C. for 16 h. LCMS indicated that the starting material was completely consumed, and the desired mass was detected. The reaction mixture was poured into H₂O (5.0 mL) and extracted with EtOAc (5.0 mL×3). The combined organic layers were washed with brine (5.0 mL×3), dried over Na₂SO₄, filtered, and concentrated under vacuum to give a residue which was purified by preparative HPLC (Phenomenex Luna column (80×30 mm, 3 μm); flow rate: 25 mL/min; gradient: 10%-35% B over 8 min; mobile phase A: 0.04% aqueous HCl, mobile phase B: acetonitrile). N-(1-(Benzofuran-4-yl)cyclopropyl)-5-(2-(dimethylamino)ethoxy)-2-methyl benzamide (69.0 mg, 182 μmol, 29% yield, HCl salt) was obtained as a white solid. M+H⁺=379.1 (LCMS); ¹H NMR (400 MHZ, DMSO-d₆) δ 10.63 (br s, 1H), 9.18 (s, 1H), 8.00 (d, J=2.0 Hz, 1H), 7.46 (d, J=8.0 Hz, 1H), 7.35-7.29 (m, 2H), 7.26-7.21 (m, 1H), 7.12 (d, J=8.4 Hz, 1H), 6.96-6.92 (m, 1H), 6.82 (d, J=2.6 Hz, 1H), 4.33 (t, J=4.9 Hz, 2H), 3.50-3.42 (m, 2H), 2.81 (d, J=4.9 Hz, 6H), 2.07 (s, 3H), 1.25 (s, 4H).

### Example 212: N-(1-(Benzo[b]thiophen-7-yl)cyclopropyl)-5-(2-(dimethylamino)ethoxy)-2-methylbenzamide (Compound 368)

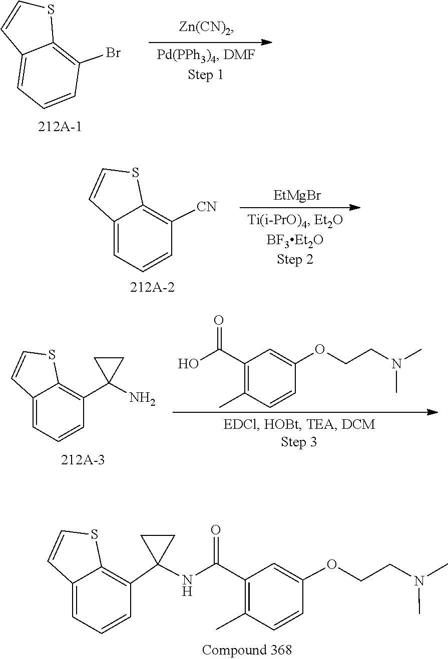 [SMILES: "EtMgBr" ; Brc1cccc2ccsc12 ; N#Cc1cccc2ccsc12 ; NC1(c2cccc3ccsc23)CC1 ; Cc1ccc(OCCN(C)C)cc1C(=O)O ; Cc1ccc(OCCN(C)C)cc1C(=O)NC1(c2cccc3ccsc23)CC1]

### Step 1: Benzo[b]thiophene-7-carbonitrile (212A-2)

To a solution of 7-bromobenzo[b]thiophene (200 mg, 939 μmol, 1.0 eq) in DMF (4.0 mL) were added Zn(CN)₂ (77.2 mg, 657 μmol, 41.7 μL, 0.7 eq) and Pd(PPh₃)₄ (108 mg, 93.9 μmol, 0.1 eq) at 25° C. under a N₂ atmosphere. The mixture was stirred at 120° C. for 3 h. TLC indicated that the starting material was completely consumed. The reaction mixture was allowed to cool to room temperature, poured into H₂O (5.0 mL) and extracted with EtOAc (5.0 mL×3). The combined organic layers were washed with brine (5.0 mL×3), dried over Na₂SO₄, filtered, and concentrated under vacuum to give a residue which was purified by preparative TLC (EtOAc/petroleum ether=1/5, R_{f}=0.4). Benzo[b]thiophene-7-carbonitrile (140 mg, 879 μmol, 94% yield) was obtained as a white solid. M+H⁺=160.0 (LCMS); ¹H NMR (400 MHZ, DMSO-d₆) δ 8.25 (d, J=8.1 Hz, 1H), 8.06-7.91 (m, 2H), 7.69-7.53 (m, 2H).

### Step 2: 1-(Benzo[b]thiophen-7-yl)cyclopropanamine (212A-3)

A mixture of benzo[b]thiophene-7-carbonitrile (140 mg, 879 μmol, 1.0 eq) in anhydrous Et₂O (15 mL) was degassed and purged with N₂ three times. To this mixture was added Ti(i-PrO)₄ (250 mg, 879 μmol, 260 μL, 1.0 eq) slowly at −78° C. and then EtMgBr (3 M in Et₂O, 645 μL, 2.2 eq) was added dropwise to maintain the temperature between −78° C. and −75° C. under a N₂ atmosphere. After the addition was complete, the resulting mixture was stirred at the same temperature for 10 min and warmed to 20° C. over 1 h. BF₃·Et₂O (250 mg, 1.76 mmol, 217 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (8.0 mL) and MTBE (8.0 mL) and extracted with MTBE (8.0 mL×4). The aqueous layer was basified to pH 8 using NaOH (2 M aqueous) and a precipitate was formed. The mixture was filtered through a pad of Celite and the slurry was washed with DCM several times. The combined filtrate was extracted with DCM (10 mL×4). The combined organic layers were dried over Na₂SO₄, filtered, and concentrated under vacuum. 1-(Benzo[b]thiophen-7-yl)cyclopropane amine (80 mg, 423 μmol, 48% yield) was obtained as a yellow gum, which was used in the next step without any further purification. M+H⁺=190.0 (LCMS); ¹H NMR (400 MHZ, DMSO-d₆) δ 7.79-7.67 (m, 2H), 7.47 (d, J=5.5 Hz, 1H), 7.34-7.25 (m, 2H), 0.96 (t, J=2.3 Hz, 2H), 0.92 (t, J=2.3 Hz, 2H).

### Step 3: N-(1-(Benzo[b]thiophen-7-yl)cyclopropyl)-5-(2-(dimethylamino)ethoxy)-2-methylbenzamide (Compound 368)

To a solution of 5-(2-(dimethylamino)ethoxy)-2-methylbenzoic acid (113 mg, 507 μmol, 1.2 eq) in DCM (3.0 mL) were added TEA (128 mg, 1.27 mmol, 176 μL, 3.0 eq), EDCI (122 mg, 634 μmol, 1.5 eq), HOBt (85.7 mg, 634 μmol, 1.5 eq) and 1-(benzo[b]thiophen-7-yl)cyclopropanamine (80.0 mg, 423 μmol, 1.0 eq). The mixture was stirred at 25° C. for 16 h. LCMS indicated that the starting material was completely consumed, and the desired mass was detected. The reaction mixture was poured into H₂O (5.0 mL) and extracted with EtOAc (5.0 mL×3). The combined organic layers were washed with brine (5.0 mL×3), dried over Na₂SO₄, and concentrated under vacuum to give a residue which was purified by preparative HPLC (Phenomenex Luna column (80×30 mm, 3 μm); flow rate: 25 mL/min; gradient: 15%-40% B over 8 min; mobile phase A: 0.04% aqueous HCl, mobile phase B: acetonitrile). N-(1-(Benzo[b]thiophen-7-yl)cyclopropyl)-5-(2-(dimethylamino)ethoxy)-2-methylbenzamide (93.0 mg, 236 μmol, 56% yield, HCl salt) was obtained as a white solid. M+H⁺=395.1 (LCMS); ¹H NMR (400 MHZ, DMSO-d₆) δ 10.65 (br s, 1H), 9.13 (s, 1H), 7.82-7.73 (m, 2H), 7.53-7.44 (m, 2H), 7.35 (t, J=7.6 Hz, 1H), 7.12 (d, J=8.5 Hz, 1H), 6.99-6.91 (m, 1H), 6.89 (d, J=2.6 Hz, 1H), 4.48-4.27 (m, 2H), 3.62-3.34 (m, 2H), 2.81 (d, J=4.9 Hz, 6H), 2.08 (s, 3H), 1.34-1.23 (m, 4H).

### Example 213: N-(1-(Benzo[b]thiophen-4-yl)cyclopropyl)-5-(2-(dimethylamino)ethoxy)-2-methylbenzamide (Compound 356)

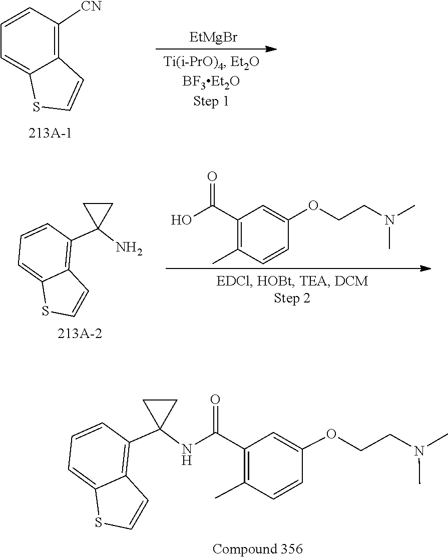 [SMILES: "EtMgBr" ; N#Cc1cccc2sccc12 ; NC1(c2cccc3sccc23)CC1 ; Cc1ccc(OCCN(C)C)cc1C(=O)O ; Cc1ccc(OCCN(C)C)cc1C(=O)NC1(c2cccc3sccc23)CC1]

### Step 1: 1-(Benzo[b]thiophen-4-yl)cyclopropanamine (213A-2)

A mixture of benzo[b]thiophene-4-carbonitrile (200 mg, 1.26 mmol, 1.0 eq) in anhydrous Et₂O (15 mL) was degassed and purged with N₂ three times. The mixture was stirred at −78° C. To this mixture was added Ti(i-PrO)₄ (357 mg, 1.26 mmol, 371 μL, 1.0 eq) slowly and then EtMgBr (3 M in Et₂O, 921 μL, 2.2 eq) was added dropwise over 1 h to maintain the temperature between −78° C. and −75° C. under a N₂ atmosphere. After the addition was complete, the resulting mixture was stirred at the same temperature for 10 min and warmed to 20° C. over 1 h. BF₃·Et₂O (357 mg, 2.51 mmol, 310 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (8.0 mL) and MTBE (8.0 mL) and extracted with MTBE (8.0 mL×4). The aqueous layer was basified to pH 8 using NaOH (2 M aqueous) and a precipitate was formed. The mixture was filtered through a pad of Celite and the slurry was washed with DCM several times. The combined filtrate was extracted with DCM (10 mL×4). The combined organic layers were dried over Na₂SO₄, filtered, and concentrated under vacuum to give the crude product, 1-(benzo[b]thiophen-4-yl)cyclopropanamine (100 mg), which was used in the next step without any further purification. M+H⁺=173.0 (LCMS); ¹H NMR (400 MHZ, DMSO-d₆) δ 7.89-7.83 (m, 1H), 7.79 (s, 2H), 7.32-7.25 (m, 2H), 0.99-0.93 (m, 2H), 0.86-0.79 (m, 2H).

### Step 2: N-(1-(Benzo[b]thiophen-4-yl)cyclopropyl)-5-(2-(dimethylamino)ethoxy)-2-methylbenzamide (Compound 356)

To a solution of 5-(2-(dimethylamino)ethoxy)-2-methylbenzoic acid (142 mg, 634 μmol, 1.2 eq) in DCM (3.0 mL) were added TEA (160 mg, 1.58 mmol, 221 μL, 3.0 eq), EDCI (152 mg, 792 μmol, 1.5 eq), HOBt (107 mg, 792 μmol, 1.5 eq) and 1-(benzo[b]thiophen-4-yl)cyclopropanamine (100 mg, 528 μmol, 1.0 eq). The mixture was stirred at 25° C. for 16 h. LCMS indicated that the starting material was completely consumed, and the desired mass was detected. The reaction mixture was poured into water (5.0 mL) at 25° C. and extracted with EtOAc (5.0 mL×3). The combined organic layers were washed with brine (5.0 mL×3), dried over Na₂SO₄, filtered, and concentrated under vacuum to give a residue which was purified by preparative HPLC (Phenomenex Luna column (80×30 mm, 3 μm); flow rate: 25 mL/min; gradient: 15%-40% B over 8 min; mobile phase A: 0.04% aqueous HCl, mobile phase B: acetonitrile). N-(1-(Benzo[b]thiophen-4-yl)cyclopropyl)-5-(2-(dimethylamino)ethoxy)-2-methylbenzamide (8.90 mg, 22.6 μmol, 4% yield, HCl salt) was obtained as a white solid. M+H⁺=395.1 (LCMS); ¹H NMR (400 MHZ, DMSO-d₆) δ 10.15-9.99 (m, 1H), 9.15 (s, 1H), 8.03 (d, J=5.5 Hz, 1H), 7.90 (d, J=8.1 Hz, 1H), 7.82 (d, J=5.5 Hz, 1H), 7.56 (d, J=7.3 Hz, 1H), 7.31 (t, J=7.7 Hz, 1H), 7.10 (d, J=8.4 Hz, 1H), 6.93-6.90 (m, 1H), 6.72 (d, J=2.3 Hz, 1H), 4.26 (br t, J=4.9 Hz, 2H), 3.48-3.42 (m, 2H), 2.80 (d, J=4.8 Hz, 6H), 2.01 (s, 3H), 1.30-1.25 (m, 2H), 1.19-1.14 (m, 2H).

### Example 214: 5-(2-(Dimethylamino)ethoxy)-2-methyl-N-(1-(2-methylbenzo[d]thiazol-7-yl)cyclopropyl)benzamide (Compound 403)

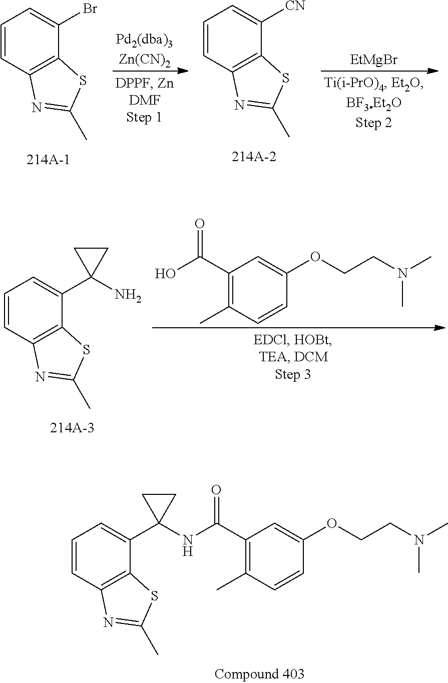

### Step 1: 2-Methylbenzo[d]thiazole-7-carbonitrile (214A-2)

To a mixture of 7-bromo-2-methylbenzo[d]thiazole (500 mg, 2.20 mmol, 1.0 eq) in DMF (6.0 mL) was degassed and purged with N₂ three times. To the mixture were added Zn(CN)₂ (386 mg, 3.28 mmol, 1.5 eq), Zn (14.3 mg, 0.22 mmol, 0.1 eq), Pd₂ (dba) 3 (100 mg, 0.11 mmol, 0.05 eq) and DPPF (122 mg, 0.22 mmol, 0.1 eq). The resulting mixture was stirred at 140° C. for 16 h under a N₂ atmosphere. LCMS indicated that the starting material was completely consumed, and the desired mass was detected. The reaction mixture was allowed to cool to room temperature, poured into H₂O (6.0 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/4. 2-Methylbenzo[d]thiazole-7-carbonitrile (300 mg, 1.72 mmol, 79% yield) was obtained as a white solid. M+H⁺=175.0 (LCMS); ¹H NMR (400 MHz, CDCl₃) δ 8.17 (dd, J=0.9, 8.2 Hz, 1H), 7.71 (dd, J=0.9, 7.6 Hz, 1H), 7.49-7.59 (m, 1H), 2.91 (s, 3H)

### Step 2: 1-(2-Methylbenzo[d]thiazol-7-yl)cyclopropanamine (214A-3)

To a mixture of 2-methylbenzo[d]thiazole-7-carbonitrile (200 mg, 1.15 mmol, 1.0 eq) in anhydrous Et₂O (10 mL) was degassed and purged with N₂ three times. The mixture was stirred at −78° C. To this mixture was added Ti(i-PrO)₄ (489 mg, 1.72 mmol, 510 μL, 1.5 eq) slowly, and then EtMgBr (3 M in Et₂O 840 μL, 2.2 eq) was added dropwise to maintain the temperature between −78° C. and −75° C. over 5 min under a N₂ atmosphere. After the addition was complete, the resulting mixture was stirred at the same temperature for 10 min and warmed to 20° C. over 1 h. BF₃·Et₂O (326 mg, 2.30 mmol, 280 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (10 mL) and MTBE (10 mL), and extracted with MTBE (10 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (10 mL×2). The combined organic layers were dried over Na₂SO₄, filtered, and concentrated under vacuum to give the crude product 1-(2-methylbenzo[d]thiazol-7-yl)cyclopropanamine (80.0 mg) as a yellow oil. M+H⁺=205.0 (LCMS);

### Step 3: 5-(2-(Dimethylamino)ethoxy)-2-methyl-N-(1-(2-methylbenzo[d]thiazol-7-yl)cyclopropyl)benzamide (Compound 403)

To a solution of 1-(2-methylbenzo[d]thiazol-7-yl)cyclopropanamine (50.0 mg, 245 μmol, 1.0 eq) and 5-(2-(dimethylamino)ethoxy)-2-methylbenzoic acid (54.6 mg, 245 μmol, 1.0 eq) in DCM (3.0 mL) were added TEA (49.5 mg, 0.49 mmol, 68.1 μL, 2.0 eq), EDCI (70.4 mg, 370 μmol, 1.5 eq) and HOBt (49.6 mg, 370 μmol, 1.5 eq). The mixture was stirred at 20° C. for 2 h. LCMS indicated that the starting material was completely consumed, and the desired mass was detected. The reaction mixture was poured into H₂O (3.0 mL) and extracted with EtOAc (3.0 mL×2). The combined organic layers were dried over Na₂SO₄, filtered, and concentrated under vacuum to give a residue which was purified by preparative HPLC (Phenomenex Gemini C18 column (75×30 mm, 3 μm); flow rate: 25 mL/min; gradient: 20%-50% B over 8 min; mobile phase A: 0.04% aqueous HCl, mobile phase B: acetonitrile). 5-(2-(Dimethylamino)ethoxy)-2-methyl-N-(1-(2-methylbenzo[d]thiazol-7-yl)cyclopropyl)benzamide (10.0 mg, 22.2 μmol, 9% yield, HCl salt) was obtained as a white solid. M+H⁺=410.1 (LCMS); ¹H NMR (400 MHZ, DMSO-d₆) δ 7.71-7.86 (m, 1H), 7.37-7.48 (m, 2H), 7.08-7.23 (m, 1H), 6.80-7.02 (m, 2H), 4.33 (br d, J=4.25 Hz, 2H), 3.49 (br t, J=4.9 Hz, 2H), 2.71-2.92 (m, 9H), 2.01-2.18 (m, 3H), 1.29 (br d, J=5.9 Hz, 4H).

### Example 215: N-(1-(Benzo[b]thiophen-3-yl)cyclopropyl)-5-(2-(dimethylamino)ethoxy)-2-methylbenzamide (Compound 346)

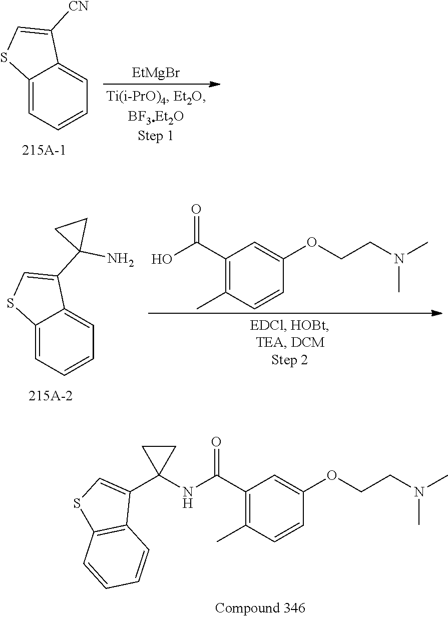 [SMILES: "EtMgBr" ; N#Cc1csc2ccccc12 ; NC1(c2csc3ccccc23)CC1 ; Cc1ccc(OCCN(C)C)cc1C(=O)O ; Cc1ccc(OCCN(C)C)cc1C(=O)NC1(c2csc3ccccc23)CC1]

### Step 1: 1-(Benzo[b]thiophen-3-yl)cyclopropanamine (215A-2)

To a mixture of benzo[b]thiophene-3-carbonitrile (300 mg, 1.88 mmol, 1.0 eq) in anhydrous Et₂O (30 mL) was degassed and purged with N₂ three times. The mixture was stirrer at −78° C. To this mixture was added Ti(i-PrO)₄ (589 mg, 2.07 mmol, 610 μL, 1.1 eq) slowly, and then EtMgBr (3 M in Et₂O, 1.38 mL, 2.2 eq) was added dropwise to maintain the temperature between −78° C. and −75° C. over 5 min under a N₂ atmosphere. After the addition was complete, the resulting mixture was stirred at the same temperature for 10 min and warmed to 20° C. over 1 h. BF₃·Et₂O (535 mg, 3.77 mmol, 470 μL, 2.0 eq) was added slowly with no obvious temperature change. The mixture was stirred at 20° C. for another 1 h. TLC indicated that the starting material was completely consumed, and a main spot was detected. The reaction mixture was added into a mixture of HCl (1 M aqueous) (30 mL) and MTBE (30 mL), and extracted with MTBE (30 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (30 mL×2). The combined organic layers were dried over Na₂SO₄, filtered, and concentrated under vacuum to give a residue which was purified by preparative TLC (DCM/MeOH=10/1, R_{f}=0.3). 1-(Benzo[b]thiophen-3-yl)cyclopropanamine (120 mg, 0.63 mmol, 34% yield) was obtained as a white solid. M+H⁺=190.0 (LCMS); ¹H NMR (400 MHZ, DMSO-d₆) δ 8.07-8.16 (m, 1H), 7.96 (d, J=7.50 Hz, 1H), 7.52 (s, 1H), 7.33-7.46 (m, 2H), 3.06 (br s, 2H), 0.91-0.99 (m, 2H), 0.84-0.91 (m, 2H).

### Step 2: N-(1-(Benzo[b]thiophen-3-yl)cyclopropyl)-5-(2-(dimethylamino)ethoxy)-2-methylbenzamide (Compound 346)

To a solution of 1-(benzo[b]thiophen-3-yl)cyclopropanamine (90.0 mg, 476 μmol, 1.0 eq) and 5-(2-(dimethylamino)ethoxy)-2-methylbenzoic acid (106 mg, 476 μmol, 1.0 eq) in DCM (3 mL) were added TEA (96.2 mg, 950 μmol, 132 μL, 2.0 eq), EDCI (137 mg, 710 μmol, 1.5 eq) and HOBt (96.4 mg, 710 μmol, 1.5 eq). The mixture was stirred at 20° C. for 2 h. LCMS indicated that the starting material was completely consumed, and the desired mass was detected. The reaction mixture was poured into H₂O (3.0 mL) and extracted with EtOAc (3.0 mL×2). The combined organic layers were dried over Na₂SO₄, filtered, and concentrated under vacuum to give a residue which was purified by preparative HPLC (Phenomenex Gemini C18 column (80×40 mm, 3 μm); flow rate: 40 mL/min; gradient: 10%-50% B over 7 min; mobile phase A: 0.04% aqueous HCl, mobile phase B: acetonitrile). N-(1-(Benzo[b]thiophen-3-yl)cyclopropyl)-5-(2-(dimethylamino)ethoxy)-2-methylbenzamide (40.0 mg, 92.6 μmol, 20% yield, HCl salt) was obtained as a yellow oil. M+H⁺=395.1 (LCMS); ¹H NMR (400 MHZ, DMSO-d₆) δ 10.73 (br s, 1H), 9.16 (s, 1H), 8.32 (d, J=7.88 Hz, 1H), 7.95 (d, J=7.88 Hz, 1H), 7.67 (s, 1H), 7.31-7.50 (m, 2H), 7.04-7.16 (m, 1H), 6.87-6.97 (m, 1H), 6.72-6.81 (m, 1H), 4.31 (t, J=5.07 Hz, 2H), 3.45 (q, J=5.04 Hz, 2H), 2.79 (d, J=4.88 Hz, 6H), 2.03 (s, 3H), 1.13-1.28 (m, 4H).

### Example 216: 5-(2-(Dimethylamino)ethoxy)-2-methyl-N-(1-(1-methyl-1H-indol-3-yl)cyclopropyl)benzamide (Compound 354)

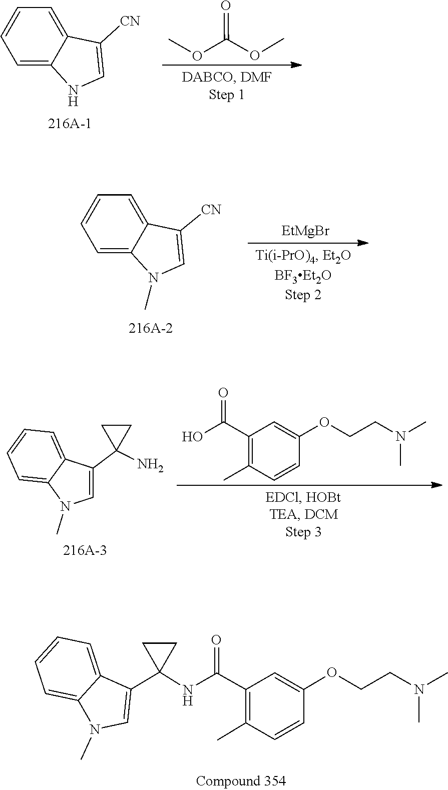

### Step 1: 1-Methyl-1H-indole-3-carbonitrile (216A-2)

To a solution of 1H-indole-3-carbonitrile (2.00 g, 14.7 mmol, 1.0 eq) in DMF (20 mL) were added DABCO (316 mg, 2.81 mmol, 309 μL, 0.2 eq) and dimethyl carbonate (21.4 g, 238 mmol, 20.0 mL, 16.9 eq). The mixture was stirred at 95° C. for 16 h. LCMS indicated that the starting material was completely consumed, and the desired mass was detected. The reaction mixture was poured into H₂O (30 mL) and extracted with EtOAc (30 mL×3). The combined organic layers were dried over Na₂SO₄, filtered, and concentrated under vacuum to give a residue which was purified by flash silica gel chromatography using a gradient of EtOAc/petroleum ether from 0/1 to 4/1. 1-Methyl-1H-indole-3-carbonitrile (2.00 g) was obtained as a brown oil. M+H⁺=157.1 (LCMS); ¹H NMR (400 MHZ, DMSO-d₆) δ 8.24 (s, 1H), 7.74-7.58 (m, 2H), 7.47-7.20 (m, 2H), 3.87 (s, 3H).

### Step 2: 1-(1-Methyl-1H-indol-3-yl)cyclopropanamine (216A-3)

A mixture of 1-methyl-1H-indole-3-carbonitrile (500 mg, 3.20 mmol, 1.0 eq) in anhydrous Et₂O (35 mL) was degassed and purged with N₂ three times. To this mixture was added Ti(i-PrO)₄ (1.00 g, 3.52 mmol, 1.04 mL, 1.1 eq) slowly at −78° C., and then EtMgBr (3 M in Et₂O, 2.35 mL, 2.2 eq) was added dropwise to maintain the temperature between −78° C. and −75° C. over 5 min under a N₂ atmosphere. After the addition was complete, the resulting mixture was stirred at the same temperature for 10 min and warmed to 20° C. over 1 h. BF₃·Et₂O (909 mg, 6.40 mmol, 790 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (30 mL) and MTBE (30 mL), and extracted with MTBE (30 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (30 mL×2). The combined organic layers were dried over Na₂SO₄, filtered, and concentrated under vacuum to give a residue which was purified by flash silica gel chromatography using a gradient of EtOAc/petroleum ether from 0/1 to 1/0. 1-(1-Methyl-1H-indol-3-yl)cyclopropanamine (80.0 mg) was obtained as a brown oil. M−17+H⁺=170.1 (LCMS).

### Step 3: 5-(2-(Dimethylamino)ethoxy)-2-methyl-N-(1-(1-methyl-1H-indol-3-yl)cyclopropyl)benzamide (Compound 354)

To a solution of 1-(1-methyl-1H-indol-3-yl)cyclopropanamine (50.0 mg, 268 μmol, 1.0 eq) and 5-(2-(dimethylamino)ethoxy)-2-methylbenzoic acid (59.9 mg, 268 μmol, 1.0 eq) in DCM (4.0 mL) were added TEA (81.5 mg, 805 μmol, 112 μL, 3.0 eq), EDCI (129 mg, 671 μmol, 2.5 eq) and HOBt (90.7 mg, 671 μmol, 2.5 eq). The mixture was stirred at 20° C. for 16 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 20%-50% B over 8 min; mobile phase A: 0.2% aqueous FA, mobile phase B: acetonitrile). 5-(2-(Dimethylamino)ethoxy)-2-methyl-N-(1-(1-methyl-1H-indol-3-yl)cyclopropyl)benzamide (13.8 mg, 30.7 μmol, 11% yield, FA salt) was obtained as a white solid. M+H⁺=392.1 (LCMS); ¹H NMR (400 MHZ, DMSO-d₆) δ 8.93 (s, 1H), 8.17 (s, 1H), 7.88 (d, J=7.9 Hz, 1H), 7.36 (d, J=8.3 Hz, 1H), 7.23 (s, 1H), 7.13 (t, J=7.6 Hz, 1H), 7.07 (d, J=8.4 Hz, 1H), 7.04-6.98 (m, 1H), 6.85 (dd, J=2.8, 8.4 Hz, 1H), 6.74 (d, J=2.6 Hz, 1H), 4.00 (t, J=5.8 Hz, 2H), 3.73 (s, 3H), 2.63 (t, J=5.8 Hz, 2H), 2.26-2.20 (m, 6H), 2.11 (s, 3H), 1.19-1.08 (m, 4H).

### Example 217: 5-(2-(Dimethylamino)ethoxy)-2-methyl-N-(1-(1-phenyl-1H-indol-3-yl)cyclopropyl)benzamide (Compound 395)

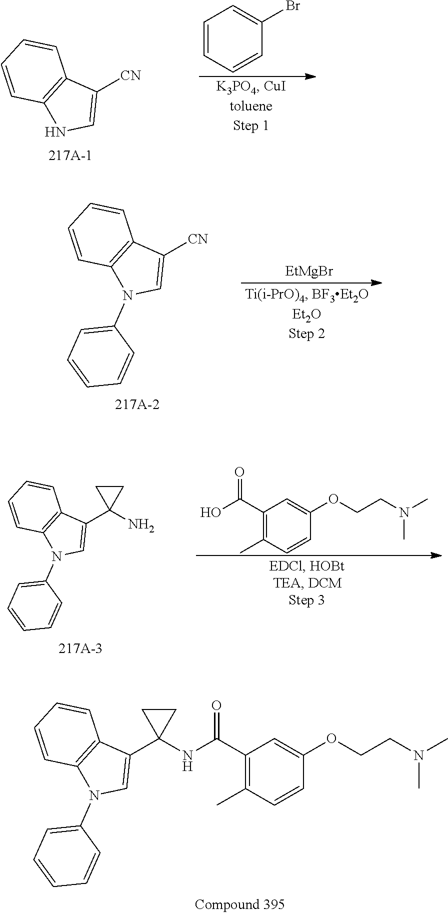

### Step 1: 1-Phenyl-1H-indole-3-carbonitrile (217A-2)

To a solution of 1H-indole-3-carbonitrile (150 mg, 1.06 mmol, 1.0 eq) in toluene (3.0 mL) were added CuI (20.1 mg, 105 μmol, 0.1 eq), K₃PO₄ (470 mg, 2.22 mmol, 2.1 eq), bromobenzene (198 mg, 1.27 mmol, 133 μL, 1.2 eq) and N,N′-dimethylethane-1,2-diamine (18.6 mg, 211 μmol, 22.7 μL, 0.2 eq) at 25° C. under a N₂ atmosphere. The mixture was stirred at 110° C. for 24 h under a N₂ atmosphere. LCMS indicated that the starting material was completely consumed, and the desired mass was detected. The reaction mixture was allowed to cool to room temperature, poured into H₂O (5.0 mL) and extracted with EtOAc (5.0 mL×3). The combined organic layers were washed with brine (5.0 mL×3) and dried over Na₂SO₄, filtered, and concentrated under vacuum to give a residue which was purified by flash silica gel chromatography using a gradient of EtOAc/petroleum ether from 0/1 to 1/2. 1-Phenyl-1H-indole-3-carbonitrile (200 mg, 916 μmol, 87% yield) was obtained as a white solid. M+H⁺=219.1 (LCMS).

### Step 2: 1-(1-Phenyl-1H-indol-3-yl)cyclopropanamine (217A-3)

A mixture of 1-phenyl-1H-indole-3-carbonitrile (500 mg, 2.29 mmol, 1.0 eq) in anhydrous Et₂O (25 mL) was degassed and purged with N₂ three times. The mixture was stirred at −78° C. To this mixture was added Ti(i-PrO)₄ (977 mg, 3.44 mmol, 1.01 mL, 1.5 eq) slowly, and then EtMgBr (3 M in Et₂O, 1.68 mL, 2.2 eq) was added dropwise to maintain the temperature between −78° C. and −75° C. over 5 min under a N₂ atmosphere. After the addition was complete, the resulting mixture was stirred at the same temperature for 10 min and warmed to 20° C. over 1 h. BF₃·Et₂O (650 mg, 4.58 mmol, 565 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (20 mL) and MTBE (20 mL), and extracted with MTBE (8.0 mL×4). The aqueous layer was basified to pH 8 using NaOH (2 M aqueous), and a precipitate was formed. The mixture was filtered through a pad of Celite and the slurry was washed with DCM several times. The combined filtrate was extracted with DCM (10 mL×4). The combined organic layers were dried over Na₂SO₄, filtered, and concentrated under vacuum to give a residue which was purified by flash silica gel chromatography using a gradient of EtOAc/petroleum ether from 0/1 to 1/2. 1-(1-Phenyl-1H-indol-3-yl)cyclopropanamine (130 mg, 523 μmol, 23% yield) was obtained as a white solid. M+H⁺=249.1 (LCMS); ¹H NMR (400 MHZ, DMSO-d₆) δ 7.78 (br d, J=7.3 Hz, 1H), 7.64-7.42 (m, 7H), 7.21-7.07 (m, 2H), 0.98 (br s, 2H), 0.87 (br s, 2H).

### Step 3: 5-(2-(Dimethylamino)ethoxy)-2-methyl-N-(1-(1-phenyl-1H-indol-3-yl)cyclopropyl)benzamide (Compound 395)

To a solution of 1-(1-phenyl-1H-indol-3-yl)cyclopropanamine (70.0 mg, 282 μmol, 1.0 eq) and 5-(2-(dimethylamino)ethoxy)-2-methylbenzoic acid (75.5 mg, 338 μmol, 1.2 eq) in DCM (3.0 mL) were added TEA (85.6 mg, 846 mmol, 118 μL, 3.0 eq), EDCI (81.1 mg, 423 μmol, 1.5 eq) and HOBt (57.1 mg, 423 mmol, 1.5 eq). The mixture was stirred at 20° C. for 16 h. LCMS indicated that the starting material was completely consumed, and the desired mass was detected. The reaction mixture was poured into H₂O (3.0 mL) and extracted with DCM (3.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 15%-45% B over 8 min; mobile phase A: 0.2% aqueous FA, mobile phase B: acetonitrile). 5-(2-(Dimethylamino)ethoxy)-2-methyl-N-(1-(1-phenyl-1H-indol-3-yl)cyclopropyl)benzamide (41.2 mg, 82.5 μmol, 29% yield, FA salt) was obtained as a white solid. M+H⁺=454.1 (LCMS); ¹H NMR (400 MHZ, DMSO-d₆) δ 9.05 (s, 1H), 8.21 (s, 1H), 8.02 (d, J=7.9 Hz, 1H), 7.62-7.52 (m, 6H), 7.44-7.32 (m, 1H), 7.24-7.17 (m, 1H), 7.16-7.11 (m, 1H), 7.08 (d, J=8.4 Hz, 1H), 6.86 (dd, J=2.7, 8.4 Hz, 1H), 6.77 (d, J=2.7 Hz, 1H), 4.00 (t, J=5.8 Hz, 2H), 2.59 (t, J=5.8 Hz, 2H), 2.20 (s, 6H), 2.13 (s, 3H), 1.24 (br d, J=3.5 Hz, 2H), 1.20 (br d, J=3.8 Hz, 2H).

### Example 218: 5-(2-(Dimethylamino)ethoxy)-2-methyl-N-(1-(quinolin-5-yl)cyclopropyl)benzamide (Compound 301)

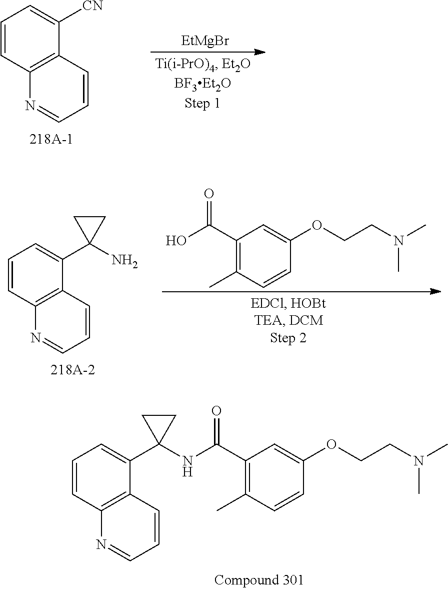 [SMILES: "EtMgBr" ; N#Cc1cccc2ncccc12 ; NC1(c2cccc3ncccc23)CC1 ; Cc1ccc(OCCN(C)C)cc1C(=O)O ; Cc1ccc(OCCN(C)C)cc1C(=O)NC1(c2cccc3ncccc23)CC1]

### Step 1: 1-(Quinolin-5-yl)cyclopropanamine (218A-2)

A mixture of quinoline-5-carbonitrile (500 mg, 3.24 mmol, 1.0 eq) in anhydrous Et₂O (40 mL) was degassed and purged with N₂ three times. The mixture was stirred at −78° C. To this mixture was added Ti(i-PrO)₄ (1.38 g, 4.86 mmol, 1.44 mL, 1.5 eq) slowly, and then EtMgBr (3 M in Et₂O, 2.38 mL, 2.2 eq) was added dropwise to maintain the temperature between-78° C. and −75° C. over 5 min under a N₂ atmosphere. After the addition was complete, the resulting mixture was stirred at the same temperature for 10 min and warmed to 20° C. over 1 h. BF₃·Et₂O (921 mg, 6.49 mmol, 801 μL, 2.0 eq) was added slowly with no obvious temperature change. The mixture was stirred at 20° C. for another 1 h. TLC indicated that the starting material was completely consumed, and a main spot was detected. The reaction mixture was added into a mixture of HCl (1 M aqueous) (40 mL) and MTBE (40 mL), and extracted with MTBE (40 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (40 mL×2). The combined organic layers were dried over Na₂SO₄, filtered, and concentrated under vacuum to give a residue which was purified by flash silica gel chromatography using a gradient of EtOAc/petroleum ether from 0/1 to 1/0. 1-(Quinolin-5-yl)cyclopropanamine (225 mg) was obtained as a brown solid. M+H⁺=185.1 (LCMS).

### Step 2: 5-(2-(Dimethylamino)ethoxy)-2-methyl-N-(1-(quinolin-5-yl)cyclopropyl)benzamide (Compound 301)

To a solution of 1-(quinolin-5-yl)cyclopropanamine (80.0 mg, 434 μmol, 1.0 eq) and 5-(2-(dimethylamino)ethoxy)-2-methylbenzoic acid (97.0 mg, 434 μmol, 1.0 eq) in DCM (3 mL) was added TEA (87.9 mg, 868 μmol, 121 μL, 2.0 eq), EDCI (125 mg, 651 μmol, 1.5 eq) and HOBt (88.0 mg, 651 μmol, 1.5 eq). The mixture was stirred at 20° C. for 2 h. LCMS indicated that the starting material was completely consumed, and the desired mass was detected. The reaction mixture was poured into H₂O (3.0 mL) and extracted with DCM (3.0 mL×2). The combined organic layers were dried over Na₂SO₄, filtered, and concentrated under vacuum to give a residue which was purified by preparative HPLC (Phenomenex Gemini C18 column (75×30 mm, 3 μm); flow rate: 25 mL/min; gradient: 15%-40% B over 8 min; mobile phase A: 10 mM aqueous NH₄HCO₃, mobile phase B: acetonitrile). 5-(2 (Dimethylamino)ethoxy)-2-methyl-N-(1-(quinolin-5-yl)cyclopropyl)benzamide (13.3 mg, 29.0 μmol, 7% yield) as a yellow gum. M+H⁺=390.1 (LCMS); ¹H NMR (400 MHZ, DMSO-d₆) δ 9.13 (s, 1H), 9.10-9.05 (m, 1H), 8.95-8.86 (m, 1H), 7.94 (d, J=8.4 Hz, 1H), 7.90-7.83 (m, 1H), 7.75-7.67 (m, 1H), 7.63-7.52 (m, 1H), 7.07-7.00 (m, 1H), 6.88-6.80 (m, 1H), 6.65-6.58 (m, 1H), 3.94 (t, J=5.8 Hz, 2H), 2.56-2.53 (m, 2H), 2.16 (s, 6H), 1.93 (s, 3H), 1.43-1.30 (m, 2H), 1.20 (s, 2H).

### Example 219: 5-(2-Aminoethoxy)-2-methyl-N-(1-(naphthalen-1-yl)cyclopropyl)benzamide (Compound 374)

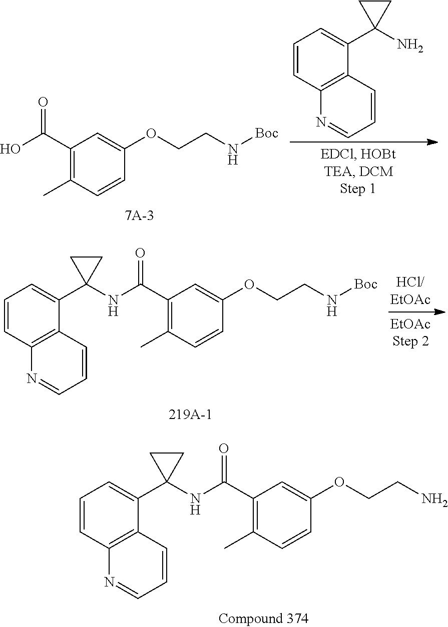 [SMILES: Cc1ccc(OCCNC(=O)OC(C)(C)C)cc1C(=O)O ; NC1(c2cccc3ncccc23)CC1 ; Cc1ccc(OCCNC(=O)OC(C)(C)C)cc1C(=O)NC1(c2cccc3ncccc23)CC1 ; Cc1ccc(OCCN)cc1C(=O)NC1(c2cccc3ncccc23)CC1]

### Step 1: tert-Butyl(2-(4-methyl-3-((1-(quinolin-5-yl)cyclopropyl)carbamoyl)phenoxy)ethyl)carbamate (219A-1)

To a solution of 5-(2-((tert-butoxycarbonyl)amino)ethoxy)-2-methylbenzoic acid (192 mg, 651 μmol, 1.2 eq) and 1-(quinolin-5-yl)cyclopropanamine (100 mg, 543 μmol, 1.0 eq) in DCM (1.0 mL) were added TEA (165 mg, 1.63 mmol, 227 μL, 3.0 eq), EDCI (156 mg, 814 μmol, 1.5 eq) and HOBt (110 mg, 814 μmol, 1.5 eq). The mixture was stirred at 20° C. for 16 h. LCMS indicated that the starting material was completely consumed, and the desired mass was detected. The reaction mixture was poured into H₂O (10 mL) and extracted with EtOAc (10 mL×2). The combined organic layers were dried over Na₂SO₄, filtered, and concentrated under vacuum to give the crude product tert-butyl(2-(4-methyl-3-((1-(quinolin-5-yl)cyclopropyl)carbamoyl)phenoxy)ethyl)carbamate (150 mg, 325 μmol, 60% yield) as a yellow oil, which was used in the next step without any further purification. M+H⁺=462.2 (LCMS).

### Step 2: 5-(2-Aminoethoxy)-2-methyl-N-(1-(quinolin-5-yl)cyclopropyl)benzamide (Compound 374)

To a stirred solution of tert-butyl(2-(4-methyl-3-((1-(quinolin-5-yl)cyclopropyl)carbamoyl) phenoxy)ethyl)carbamate (150 mg, 325 μmol, 1.0 eq) in EtOAc (1.0 mL) was added HCl/EtOAc (4 M, 4.0 mL). The mixture was stirred at 20° C. for 4 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 1%-40% B over 8 min; mobile phase A: 0.04% aqueous HCl, mobile phase B: acetonitrile). 5-(2-Aminoethoxy)-2-methyl-N-(1-(quinolin-5-yl)cyclopropyl)benzamide (120 mg, 318 μmol, 96% yield, HCl salt) was obtained as a white solid. M+H⁺=362.1 (LCMS); ¹H NMR (400 MHZ, DMSO-d₆) δ 9.88 (d, J=8.6 Hz, 1H), 9.43 (s, 1H), 9.35-9.29 (m, 1H), 8.41 (d, J=8.5 Hz, 1H), 8.34 (br s, 3H), 8.21-8.15 (m, 2H), 8.15-8.08 (m, 1H), 7.07 (d, J=8.5 Hz, 1H), 6.89 (dd, J=2.6, 8.4 Hz, 1H), 6.74 (d, J=2.6 Hz, 1H), 4.13 (t, J=5.1 Hz, 2H), 3.17-3.06 (m, 2H), 1.96 (s, 3H), 1.51-1.41 (m, 2H), 1.34-1.27 (m, 2H).

### Example 220: 2-Methyl-5-(2-(methylamino)ethoxy)-N-(1-(quinolin-5-yl)cyclopropyl)benzamide (Compound 385)

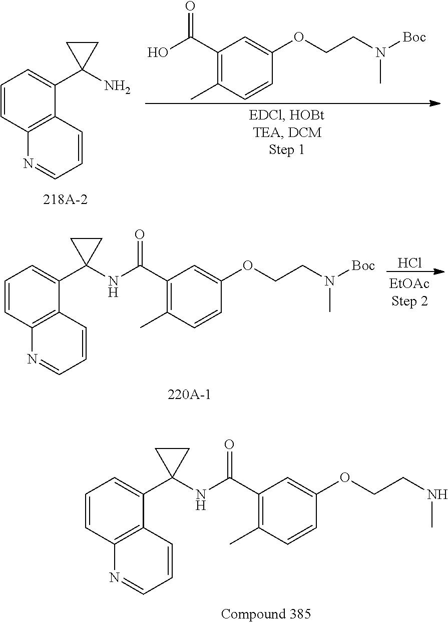 [SMILES: NC1(c2cccc3ncccc23)CC1 ; Cc1ccc(OCCN(C)C(=O)OC(C)(C)C)cc1C(=O)O ; Cc1ccc(OCCN(C)C(=O)OC(C)(C)C)cc1C(=O)NC1(c2cccc3ncccc23)CC1 ; Cl ; CNCCOc1ccc(C)c(C(=O)NC2(c3cccc4ncccc34)CC2)c1]

### Step 1: tert-Butyl methyl(2-(4-methyl-3-((1-(quinolin-5-yl)cyclopropyl)carbamoyl)phenoxy)ethyl)carbamate (220A-1)

To a solution of 1-(quinolin-5-yl)cyclopropanamine (100 mg, 543 μmol, 1.0 eq) and 5-(2-((tert-butoxycarbonyl)(methyl)amino)ethoxy)-2-methylbenzoic acid (168 mg, 543 μmol, 1.0 eq) in DCM (5.0 mL) were added TEA (165 mg, 1.63 mmol, 227 μL, 3.0 eq), EDCI (260 mg, 1.36 mmol, 2.5 eq) and HOBt (183 mg, 1.36 mmol, 2.5 eq). The mixture was stirred at 20° C. for 16 h. LCMS indicated that the starting material was completely consumed, and the desired mass was detected. The reaction mixture was poured into H₂O (10 mL) and extracted with DCM (10 mL×2). The combined organic layers were dried over Na₂SO₄, filtered, and concentrated under vacuum to give the product tert-butyl methyl(2-(4-methyl-3-((1-(quinolin-5-yl)cyclopropyl)carbamoyl)phen oxy)ethyl)carbamate (250 mg) as a brown oil which was used into the next step without further purification. M+H⁺=476.2 (LCMS).

### Step 2: 2-Methyl-5-(2-(methylamino)ethoxy)-N-(1-(quinolin-5-yl)cyclopropyl)benzamide (Compound 385)

To a solution of tert-butyl methyl(2-(4-methyl-3-((1-(quinolin-5-yl)cyclopropyl)carbamoyl) phenoxy)ethyl)carbamate (250 mg, 526 μmol, 1.0 eq) in EtOAc (3.0 mL) was added HCl/EtOAc (4 M, 10 mL). The mixture was stirred at 20° C. for 2 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 10%-30% B over 8 min; mobile phase A: 0.04% aqueous HCl, mobile phase B: acetonitrile). 2-Methyl-5-(2-(methylamino)ethoxy)-N-(1-(quinolin-5-yl)cyclopropyl)benzamide (44.4 mg, 107 μmol, 20% yield, HCl salt) was obtained as a yellow solid. M+H⁺=376.1 (LCMS); 1H NMR (400 MHZ, CD₃OD) δ 10.06 (d, J=8.5 Hz, 1H), 9.27 (dd, J=1.3, 5.4 Hz, 1H), 8.36 (dd, J=1.3, 6.9 Hz, 1H), 8.27-8.15 (m, 3H), 7.11 (d, J=8.5 Hz, 1H), 6.95 (dd, J=2.7, 8.4 Hz, 1H), 6.82 (d, J=2.6 Hz, 1H), 4.26-4.15 (m, 2H), 3.44-3.37 (m, 2H), 2.76 (s, 3H), 2.02 (s, 3H), 1.65-1.59 (m, 2H), 1.48-1.42 (m, 2H).

### Example 221: (S)-5-(Azetidin-2-ylmethoxy)-2-methyl-N-(1-(quinolin-5-yl)cyclopropyl)benzamide (Compound 381)

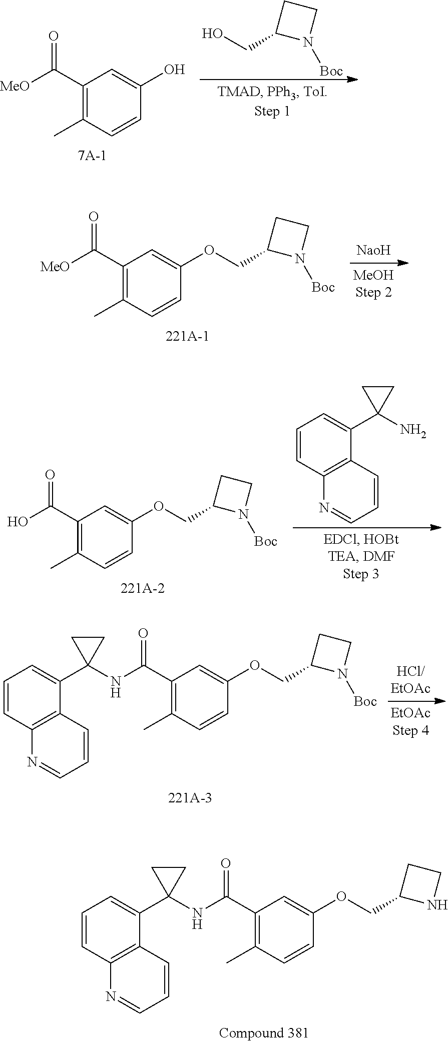

### Step 1: (S)-tert-butyl 2-((3-(methoxycarbonyl)-4-methylphenoxy)methyl)azetidine-1-carboxylate (221A-1)

To a solution of methyl 5-hydroxy-2-methylbenzoate (300 mg, 1.81 mmol, 1.0 eq) and(S)-tert-butyl 2-(hydroxymethyl)azetidine-1-carboxylate (508 mg, 2.72 mmol, 1.5 eq) in toluene (15 mL) were added TMAD (935 mg, 5.43 mmol, 3.0 eq) and PPh₃ (1.42 g, 5.43 mmol, 3.0 eq). The resulting mixture was degassed and purged with N₂ three times and then stirred at 100° C. for 16 h under a N₂ atmosphere. LCMS indicated that the starting material was completely consumed, and the desired mass was detected. The reaction mixture was allowed to cool to room temperature, poured into H₂O (15 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/5. (S)-tert-Butyl 2-((3-(methoxycarbonyl)-4-methylphenoxy)methyl)azetidine-1-carboxylate (500 mg, 1.49 mmol, 82% yield) was obtained as a white solid. M−100+H⁺=236.1 (LCMS); ¹H NMR (400 MHZ, CDCl₃) δ 7.49 (br d, J=1.5 Hz, 1H), 7.15 (br d, J=8.3 Hz, 1H), 7.00 (br dd, J=1.6, 8.3 Hz, 1H), 4.51 (br d, J=4.3 Hz, 1H), 4.28 (br s, 1H), 4.22-4.03 (m, 2H), 3.89 (s, 3H), 3.87 (br s, 1H), 2.52 (s, 3H), 2.42-2.13 (m, 2H), 1.42 (s, 9H).

### Step 2: (S)-5-((1-(tert-Butoxycarbonyl)azetidin-2-yl)methoxy)-2-methylbenzoic acid (221A-2)

To a solution of(S)-tert-butyl 2-((3-(methoxycarbonyl)-4-methylphenoxy)methyl)azetidine-1-carboxylate (500 mg, 1.49 mmol, 1.0 eq) in a mixture of MeOH (20 mL) and THF (10 mL) was added NaOH (2 M aqueous, 3.0 mL, 4.0 eq). The mixture was stirred at 70° C. for 16 h. LCMS indicated that the starting material was completely consumed, and the desired mass was detected. The reaction mixture was allowed to cool to room temperature, poured into H₂O (20 mL) and washed with petroleum ether (10 mL×3). The aqueous layer was acidified to pH 5 with HCl (1 M aqueous). The product was extracted with EtOAc (10 mL×2). The combined organic layers were dried over Na₂SO₄, filtered, and concentrated under vacuum to give crude product (S)-5-((1-(tert-butoxycarbonyl)azetidin-2-yl)methoxy)-2-methylbenzoic acid (100 mg) as a white solid. M+H⁺=318.2 (LCMS); ¹H NMR (400 MHZ, CDCl₃) δ 7.62 (d, J=2.7 Hz, 1H), 7.17 (s, 1H), 7.08 (br d, J=2.7 Hz, 1H), 4.53 (br d, J=2.0 Hz, 1H), 4.31 (br s, 1H), 4.12 (dd, J=2.9, 10.1 Hz, 1H), 4.01-3.83 (m, 2H), 2.58 (s, 3H), 2.44-2.19 (m, 2H), 1.43 (s, 9H).

### Step 3: (S)-tert-Butyl 2-((4-methyl-3-((1-(quinolin-5-yl)cyclopropyl)carbamoyl)phenoxy)methyl)azetidine-1-carboxylate (221A-3)

To a solution of(S)-5-((1-(tert-butoxycarbonyl)azetidin-2-yl)methoxy)-2-methylbenzoic acid (100 mg, 311 μmol, 1.0 eq) and 1-(quinolin-5-yl)cyclopropanamine (68.8 mg, 373 μmol, 1.2 eq) in DMF (3.0 mL) were added TEA (94.5 mg, 933 μmol, 130 μL, 3.0 eq), EDCI (89.5 mg, 467 μmol, 1.5 eq) and HOBt (63.1 mg, 466 μmol, 1.5 eq). The mixture was stirred at 20° C. for 3 h. LCMS indicated that the starting material was completely consumed, and the desired mass was detected. The reaction mixture was poured into H₂O (3.0 mL) and extracted with EtOAc (3.0 mL×3). The combined organic layers were dried over Na₂SO₄, filtered, and concentrated under vacuum to give a residue which was purified by preparative TLC (pure EtOAc, R_{f}=0.6). (S)-tert-Butyl 2-((4-methyl-3-((1-(quinolin-5-yl)cyclopropyl)carbamoyl)phenoxy)methyl) azetidine-1-carboxylate (120 mg, 246 μmol, 79% yield) was obtained as a white solid. M+H⁺=488.3 (LCMS).

### Step 4: (S)-5-(Azetidin-2-ylmethoxy)-2-methyl-N-(1-(quinolin-5-yl)cyclopropyl)benzamide (Compound 381)

To a stirred solution of(S)-tert-butyl 2-((4-methyl-3-((1-(quinolin-5-yl)cyclopropyl)carbamoyl)phenoxy)methyl)azetidine-1-carboxylate (110 mg, 226 μmol, 1.0 eq) in EtOAc (3.0 mL) was added HCl/EtOAc (4 M, 3.9 mL). The mixture was stirred at 20° C. for 1 h. LCMS indicated that the starting material was completely consumed, and the desired mass was detected. The mixture was concentrated under vacuum to give a residue which was purified by preparative HPLC (C18 column (150×30 mm, 3 μm); flow rate: 60 mL/min; gradient: 1%-40% B over 8 min; mobile phase A: 0.1% aqueous TFA, mobile phase B: acetonitrile). (S)-5-(Azetidin-2-ylmethoxy)-2-methyl-N-(1-(quinolin-5-yl)cyclopropyl)benzamide (43.4 mg, 86.5 μmol, 39% yield, TFA salt) was obtained as a white solid. M+H⁺=388.1 (LCMS); ¹H NMR (400 MHZ, DMSO-d₆) δ 9.27 (br d, J=8.3 Hz, 1H), 9.17 (s, 1H), 9.03 (dd, J=1.3, 4.3 Hz, 1H), 8.99-8.72 (m, 2H), 8.13-7.89 (m, 2H), 7.87-7.67 (m, 2H), 7.09 (d, J=8.5 Hz, 1H), 6.91 (dd, J=2.8, 8.4 Hz, 1H), 6.71 (d, J=2.6 Hz, 1H), 4.80-4.51 (m, 1H), 4.34-4.07 (m, 2H), 4.03-3.69 (m, 2H), 2.47-2.18 (m, 2H), 1.95 (s, 3H), 1.39 (s, 2H), 1.29-1.20 (m, 2H).

### Example 222: 5-(2-Aminoethoxy)-2-methyl-N-(1-(naphthalen-1-yl)cyclopropyl)benzamide (Compound 383)

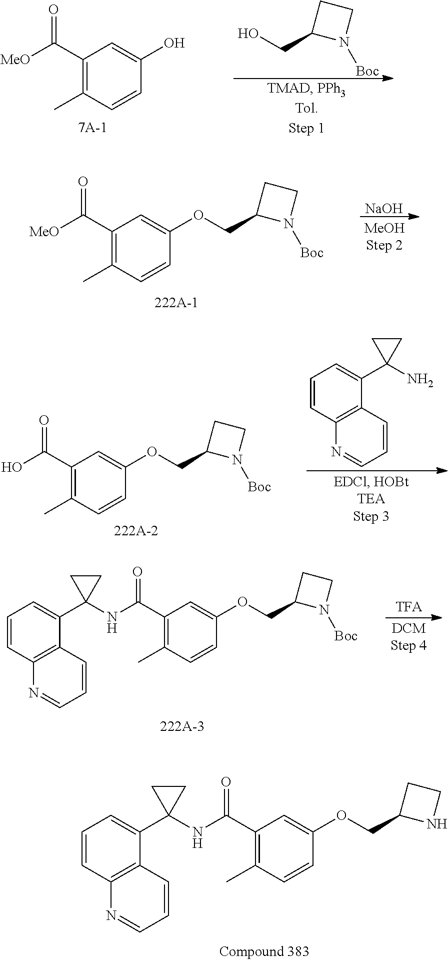

### Step 1: (R)-tert-Butyl 2-((3-(methoxycarbonyl)-4-methylphenoxy)methyl)azetidine-1-carboxylate (222A-1)

A mixture of methyl 5-hydroxy-2-methylbenzoate (500 mg, 3.01 mmol, 1.0 eq), methyl 5-hydroxy-2-methylbenzoate (845 mg, 4.51 mmol, 1.5 eq) and PPh₃ (2.37 g, 9.03 mmol, 3.0 eq) in toluene (15 mL) was degassed and purged with N₂ three times. To the mixture was added TMAD (1.55 g, 9.03 mmol, 3.0 eq) in portions at 20° C. The resulting mixture was stirred at 100° C. for 16 h under a N₂ atmosphere. LCMS indicated that the starting material was completely consumed, and the desired mass was detected. The reaction mixture was allowed to cool to room temperature, poured into H₂O (30 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/9. (R)-tert-Butyl 2-((3-(methoxycarbonyl)-4-methylphenoxy)methyl)azetidine-1-carboxylate (900 mg, 2.68 mmol, 89% yield) was obtained as a yellow oil. M+Na⁺=358.1 (LCMS); ¹H NMR (400 MHZ, DMSO-d₆) δ 7.37 (d, J=2.8 Hz, 1H), 7.24 (d, J=8.5 Hz, 1H), 7.11 (dd, J=2.8, 8.4 Hz, 1H), 4.48-4.38 (m, 1H), 4.26 (dd, J=4.6, 10.4 Hz, 1H), 4.07 (dd, J=2.9, 10.4 Hz, 1H), 3.82 (s, 3H), 3.76 (br t, J=7.4 Hz, 2H), 2.42 (s, 3H), 2.34-2.25 (m, 1H), 2.19-2.09 (m, 1H), 1.33 (s, 9H).

### Step 2: (R)-5-((1-(tert-Butoxycarbonyl)azetidin-2-yl)methoxy)-2-methylbenzoic acid (222A-2)

To a solution of (R)-tert-butyl 2-((3-(methoxycarbonyl)-4-methylphenoxy)methyl)azetidine-1-carboxylate (900 mg, 2.68 mmol, 1.0 eq) in a mixture of MeOH (2.0 mL) and THF (8.0 mL) was added NaOH (2 M aqueous, 3.00 mL, 5.0 eq). The mixture was stirred at 20° C. for 16 h. LCMS indicated that the starting material was completely consumed, and the desired mass was detected. H₂O (10 mL) was added, and the mixture was washed with MTBE (10 mL×2). The aqueous layer was acidified to pH 5 using HCl (1 M aqueous). The product was extracted with 2,5-dimethyltetrahydrofuran (10 mL×3). The combined organic layers were dried over Na₂SO₄, filtered, and concentrated under vacuum to give the crude product (R)-5-((1-(tert-butoxycarbonyl)azetidin-2-yl)methoxy)-2-methylbenzoic acid (200 mg, 622 μmol, 52% yield) as a yellow gum, which was used in the next step without any further purification. M+Na⁺=344.1 (LCMS); ¹H NMR (400 MHZ, DMSO-d₆) δ 12.50-12.40 (m, 1H), 7.36 (d, J=2.9 Hz, 1H), 7.20 (d, J=8.5 Hz, 1H), 7.07 (dd, J=2.8, 8.4 Hz, 1H), 4.47-4.38 (m, 1H), 4.24 (dd, J=4.6, 10.3 Hz, 1H), 4.06 (dd, J=2.8, 10.3 Hz, 1H), 3.75 (br t, J=7.1 Hz, 2H), 2.43 (s, 3H), 2.34-2.24 (m, 1H), 2.19-2.08 (m, 1H), 1.32 (s, 9H).

### Step 3: (R)-tert-Butyl 2-((4-methyl-3-((1-(quinolin-5-yl)cyclopropyl)carbamoyl)phenoxy)methyl)azetidine-1-carboxylate (222A-3)

To a solution of 1-(quinolin-5-yl)cyclopropanamine (86.0 mg, 467 μmol, 1.0 eq) and (R)-5-((1-(tert-butoxycarbonyl)azetidin-2-yl)methoxy)-2-methylbenzoic acid (150 mg, 467 μmol, 1.0 eq) in DMF (2.0 mL) were added TEA (142 mg, 1.40 mmol, 195 μL, 3.0 eq), EDCI (134 mg, 700 μmol, 1.5 eq) and HOBt (94.6 mg, 700 mmol, 1.5 eq). The mixture was stirred at 20° C. for 16 h. LCMS indicated that the starting material was completely consumed, and the desired mass was detected. The reaction mixture was poured into H₂O (10 mL) and extracted with EtOAc (10 mL×2). The combined organic layers were dried over Na₂SO₄, filtered, and concentrated under vacuum to give crude product (R)-tert-butyl 2-((4-methyl-3-((1-(quinolin-5-yl)cyclopropyl)carbamoyl)phenoxy)methyl)azetidine-1-carboxylate (200 mg, 410 μmol, 88% yield), which was used in the next step without any further purification. M+H⁺=488.2 (LCMS).

### Step 4: (R)-5-(Azetidin-2-ylmethoxy)-2-methyl-N-(1-(quinolin-5-yl)cyclopropyl)benzamide (Compound 383)

To a stirred solution of (R)-tert-butyl 2-((4-methyl-3-((1-(quinolin-5-yl)cyclopropyl) carbamoyl)phenoxy)methyl)azetidine-1-carboxylate (200 mg, 410 μmol, 1.0 eq) in DCM (2.0 mL) was added TFA (3.0 mL). The mixture was stirred at 20° C. for 4 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 5%-25% B over 8 min; mobile phase A: 0.1% aqueous TFA, mobile phase B: acetonitrile). (R)-5-(Azetidin-2-ylmethoxy)-2-methyl-N-(1-(quinolin-5-yl)cyclopropyl)benzamide (120 mg, 310 μmol, 76% yield, TFA salt) was obtained as a white solid. M+H⁺=388.1 (LCMS); ¹H NMR (400 MHZ, DMSO-d₆) δ 9.23 (d, J=8.5 Hz, 1H), 9.16 (s, 1H), 9.04-8.98 (m, 1H), 8.94-8.74 (m, 2H), 8.00 (d, J=8.4 Hz, 1H), 7.95 (d, J=7.1 Hz, 1H), 7.84-7.77 (m, 1H), 7.72 (dd, J=4.4, 8.5 Hz, 1H), 7.09 (d, J=8.4 Hz, 1H), 6.91 (dd, J=2.7, 8.3 Hz, 1H), 6.70 (d, J=2.6 Hz, 1H), 4.65 (br d, J=4.9 Hz, 1H), 4.23 (dd, J=7.2, 11.2 Hz, 1H), 4.18-4.10 (m, 1H), 3.98-3.89 (m, 1H), 3.83 (br dd, J=6.0, 10.3 Hz, 1H), 2.48-2.43 (m, 1H), 2.39-2.30 (m, 1H), 1.95 (s, 3H), 1.44-1.33 (m, 2H), 1.28-1.19 (m, 2H).

### Example 223: (S)-2-Methyl-5-((1-methylazetidin-2-yl)methoxy)-N-(1-(quinolin-5-yl)cyclopropyl)benzamide (Compound 391)

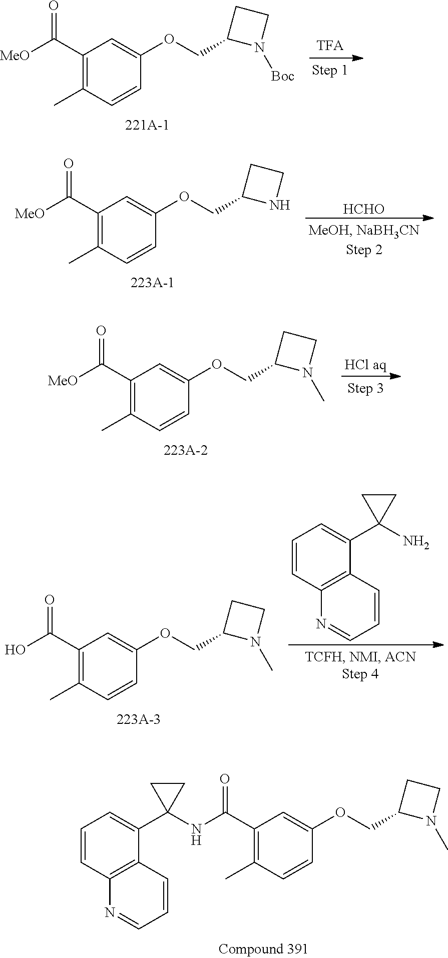

### Step 1: (S)-Methyl 5-(azetidin-2-ylmethoxy)-2-methylbenzoate (223A-1)

To a solution of(S)-tert-butyl 2-((3-(methoxycarbonyl)-4-methylphenoxy)methyl)azetidine-1-carboxylate (250 mg, 745 μmol, 1.0 eq) in DCM (3.0 mL) was added TFA (1.54 g, 13.5 mmol, 1.00 mL, 18 eq). The mixture was stirred at 20° C. for 1 h. LCMS indicated that the starting material was completely consumed, and the desired mass was detected. The mixture was concentrated under vacuum at 20° C. to give the crude product(S)-methyl 5-(azetidin-2-ylmethoxy)-2-methylbenzoate (250 mg, TFA salt) as a yellow oil. M+H⁺=236.2 (LCMS).

### Step 2: (S)-Methyl 2-methyl-5-((1-methylazetidin-2-yl)methoxy)benzoate (223A-2)

To a solution of(S)-methyl 5-(azetidin-2-ylmethoxy)-2-methylbenzoate (250 mg, 1.06 mmol, 1.0 eq) in MeOH (5.0 mL) was added TEA (10.0 μL), followed by the addition of formaldehyde (172 mg, 2.10 mmol, 158 μL, 37% purity in water, 2.0 eq). The resulting mixture was adjusted to pH 6 with a small amount of AcOH. The mixture was stirred at 20° C. for 30 min, then NaBH₃CN (134 mg, 2.13 mmol, 2.0 eq) was added. The reaction mixture was stirred at 20° C. for another 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by flash silica gel chromatography using a gradient of DCM/MeOH from 100/1 to 10/1. (S)-Methyl 2-methyl-5-((1-methyl azetidin-2-yl)methoxy)benzoate (160 mg, 642 μmol, 60% yield) was obtained as a colorless oil. M+H⁺=250.1 (LCMS).

### Step 3: (S)-2-Methyl-5-((1-methylazetidin-2-yl)methoxy)benzoic acid (223A-3)

A solution of(S)-methyl 2-methyl-5-((1-methylazetidin-2-yl)methoxy)benzoate (260 mg, 1.04 mmol, 1.0 eq) in HCl (2 M aqueous, 10 mL) was stirred at 100° C. for 16 h. LCMS indicated that the starting material was completely consumed, and the desired mass was detected. The reaction mixture was allowed to cool to room temperature, then treated with NaOH (2 M aqueous) to adjust the pH to 6. The resulting mixture was concentrated under vacuum to remove the water completely. The resulting mixture was treated with MeOH/DCM (V/V=10/1, 10 mL) then filtered. The filter cake was washed with MeOH/DCM (V/V=5.0 mL×2) to ensure all product was washed from the solids. The combined organic layers were concentrated under vacuum to give(S)-2-methyl-5-((1-methylazetidin-2-yl)methoxy)benzoic acid (160 mg, crude) as a hydrophilic, colorless oil. M+H⁺=236.1 (LCMS).

### Step 4: (S)-2-Methyl-5-((1-methylazetidin-2-yl)methoxy)-N-(1-(quinolin-5-yl)cyclopropyl)benzamide (Compound 391)

To a solution of(S)-2-methyl-5-((1-methylazetidin-2-yl)methoxy)benzoic acid (80.0 mg, 340 μmol, 1.0 eq) and 1-(quinolin-5-yl)cyclopropanamine (43.9 mg, 238 mmol, 0.7 eq) in ACN (5.0 mL) were added TCFH (95.4 mg, 340 μmol, 1.0 eq) and NMI (27.9 mg, 340 μmol, 1.0 eq). The mixture was stirred at 20° C. for 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Gemini C18 column (80×30 mm, 10 μm); flow rate: 25 mL/min; gradient: 5%-20% B over 8 min; mobile phase A: 0.04% aqueous HCl, mobile phase B: acetonitrile). (S)-2-Methyl-5-((1-methylazetidin-2-yl)methoxy)-N-(1-(quinolin-5-yl)cyclo propyl)benzamide (16.6 mg, 34.6 μmol, 10% yield, HCl salt) was obtained as a brown solid. M+H⁺=402.2 (LCMS); ¹H NMR (400 MHZ, DMSO-d₆) δ 11.03 (br s, 1H), 9.83 (br d, J=8.5 Hz, 1H), 9.41-9.36 (m, 1H), 9.31 (d, J=4.6 Hz, 1H), 8.34 (d, J=8.4 Hz, 1H), 8.21-8.06 (m, 3H), 7.09 (d, J=8.3 Hz, 1H), 6.91 (dd, J=2.6, 8.4 Hz, 1H), 6.76 (d, J=2.5 Hz, 1H), 4.70-4.57 (m, 1H), 4.42 (dd, J=8.3, 11.0 Hz, 1H), 4.22 (dd, J=3.2, 10.9 Hz, 1H), 4.06-3.92 (m, 1H), 3.84 (br dd, J=6.8, 9.4 Hz, 1H), 2.80 (d, J=5.0 Hz, 3H), 2.41-2.20 (m, 2H), 1.95 (s, 3H), 1.46 (br s, 2H), 1.34-1.27 (m, 2H).

### Example 224: (R)-2-Methyl-5-((1-methylazetidin-2-yl)methoxy)-N-(1-(quinolin-5-yl)cyclopropyl)benzamide (Compound 390)

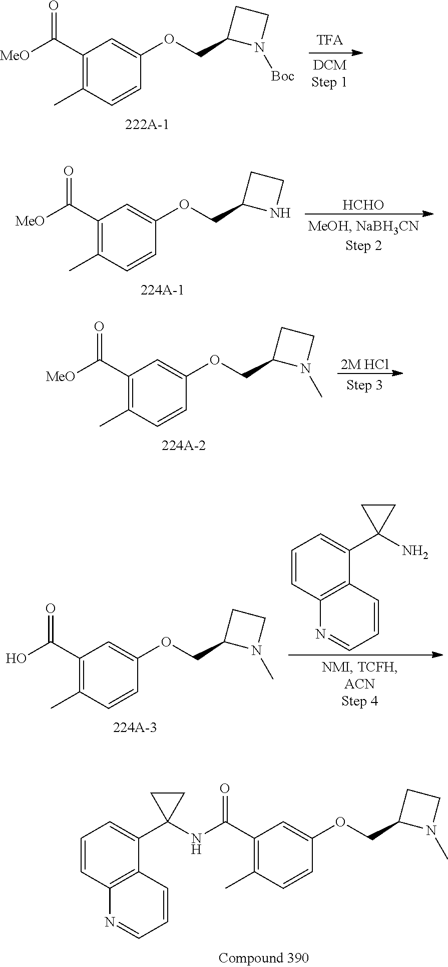

### Step 1: (R)-Methyl 5-(azetidin-2-ylmethoxy)-2-methylbenzoate (224A-1)

To a stirred solution of (R)-tert-butyl 2-((3-(methoxycarbonyl)-4-methylphenoxy)methyl) azetidine-1-carboxylate (250 mg, 745 μmol, 1.0 eq) in DCM (6.0 mL) was added TFA (2.0 mL). The mixture was stirred at 20° C. for 1 h. LCMS indicated that the starting material was completely consumed, and the desired mass was detected. The mixture was concentrated under vacuum at 30° C. to give the crude product (R)-methyl 5-(azetidin-2-ylmethoxy)-2-methylbenzoate (250 mg, TFA salt) as a colorless oil. M+H⁺=236.2 (LCMS).

### Step 2: (R)-Methyl 2-methyl-5-((1-methylazetidin-2-yl)methoxy)benzoate (224A-2)

To a solution of (R)-methyl 5-(azetidin-2-ylmethoxy)-2-methylbenzoate (250 mg, 716 μmol, 1.0 eq) in MeOH (5.0 mL) was added TEA (99.6 μL), followed by the addition of formaldehyde (116 mg, 1.43 mmol, 107 μL, 37% purity in water, 2.0 eq). The resulting mixture was adjusted to pH 6 with a small amount of AcOH. The mixture was stirred at 20° C. for 30 min, then NaBH₃CN (90.0 mg, 1.43 mmol, 2.0 eq) was added. The resulting mixture was stirred at 20° C. for another 16 h. LCMS indicated that the starting material was completely consumed, and the desired mass was detected. The reaction mixture was poured into H₂O (10 mL) and extracted with EtOAc (5.0 mL×3). The combined organic layers were dried over Na₂SO₄, filtered, and concentrated under vacuum to give a residue which was purified by preparative TLC (DCM/MeOH=10/1, R_{f}=0.5). (R)-Methyl 2-methyl-5-((1-methylazetidin-2-yl)methoxy)benzoate (100 mg, 401 μmol, 56% yield) was obtained as a colorless oil. M+H⁺=250.2 (LCMS).

### Step 3: (R)-2-Methyl-5-((1-methylazetidin-2-yl)methoxy)benzoic acid (224A-3)

A solution of (R)-methyl 2-methyl-5-((1-methylazetidin-2-yl)methoxy)benzoate (90.0 mg, 361 μmol, 1.0 eq) in HCl (2 M aqueous, 3.0 mL) was stirred at 100° C. for 16 h. LCMS indicated that the starting material was completely consumed, and the desired mass was detected. The reaction mixture was allowed to cool to room temperature, then treated with NaOH (2 M aqueous) to adjust the pH to 6. The resulting mixture was concentrated under vacuum to remove the water completely. The resulting mixture was treated with DCM/MeOH (V/V=10/1, 10 mL) then filtered. The filter cake was washed with DCM/MeOH (5.0 mL×2) to ensure all product was washed from the solids. The combined organic layers were concentrated under vacuum to give the crude product (R)-2-methyl-5-((1-methylazetidin-2-yl)methoxy)benzoic acid (40.0 mg) as a hydrophilic, colorless oil. M+H⁺=236.2 (LCMS); ¹H NMR (400 MHZ, DMSO-d₆) δ 7.12 (d, J=2.8 Hz, 1H), 6.99 (d, J=8.4 Hz, 1H), 6.76 (dd, J=2.8, 8.3 Hz, 1H), 3.90 (d, J=5.5 Hz, 2H), 3.39-3.22 (m, 3H), 2.78-2.65 (m, 1H), 2.36 (s, 3H), 2.25 (s, 3H), 2.03-1.92 (m, 1H).

### Step 4: (R)-2-Methyl-5-((1-methylazetidin-2-yl)methoxy)-N-(1-(quinolin-5-yl)cyclopropyl)benzamide (Compound 390)

To a solution of (R)-2-methyl-5-((1-methylazetidin-2-yl)methoxy)benzoic acid (40.0 mg, 170 μmol, 1.0 eq) and 1-(quinolin-5-yl)cyclopropanamine (31.3 mg, 170 mmol, 1.0 eq) in ACN (4.0 mL) were added TCFH (57.2 mg, 204 μmol, 1.2 eq) and NMI (48.9 mg, 595 μmol, 47.4 μL, 3.5 eq). The mixture was stirred at 20° C. for 16 h. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 1%-20% B over 8 min; mobile phase A: 0.04% aqueous HCl, mobile phase B: acetonitrile). (R)-2-Methyl-5-((1-methylazetidin-2-yl)methoxy)-N-(1-(quinolin-5-yl)cyclo propyl)benzamide (19.8 mg, 45.2 μmol, 27% yield, HCl salt) was obtained as a yellow solid. M+H⁺=402.1 (LCMS); ¹H NMR (400 MHZ, DMSO-d₆) δ 11.07 (br s, 1H), 9.84-9.74 (m, 1H), 9.42-9.34 (m, 1H), 9.28 (d, J=4.1 Hz, 1H), 8.32 (d, J=8.5 Hz, 1H), 8.18-8.14 (m, 1H), 8.14-8.05 (m, 2H), 7.08 (d, J=8.5 Hz, 1H), 6.91 (dd, J=2.8, 8.4 Hz, 1H), 6.76 (d, J=2.8 Hz, 1H), 4.69-4.57 (m, 1H), 4.42 (dd, J=8.2, 11.2 Hz, 1H), 4.22 (dd, J=3.3, 11.3 Hz, 1H), 4.03-3.93 (m, 1H), 3.84 (br dd, J=6.7, 9.6 Hz, 1H), 2.80 (d, J=5.0 Hz, 3H), 2.41-2.23 (m, 2H), 1.95 (s, 3H), 1.50-1.41 (m, 2H), 1.35-1.26 (m, 2H).

### Example 225: (S)-2-Methyl-5-(pyrrolidin-2-ylmethoxy)-N-(1-(quinolin-5-yl)cyclopropyl)benzamide (Compound 380)

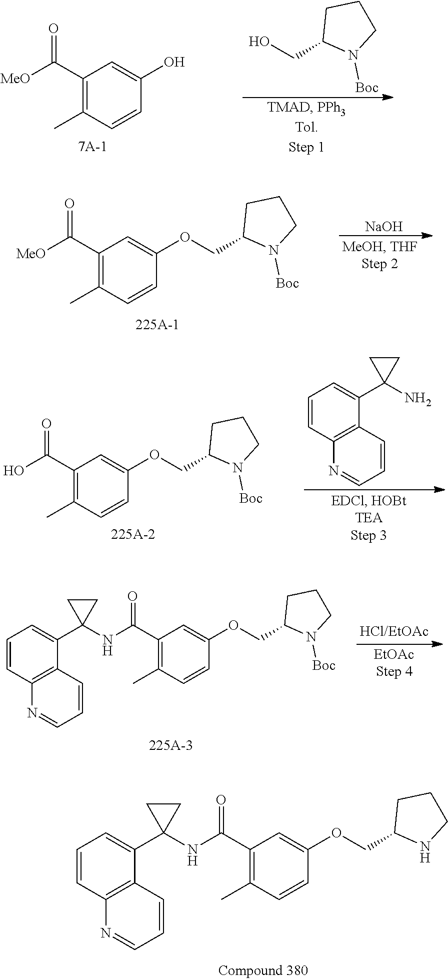

### Step 1: (S)-tert-Butyl 2-((3-(methoxycarbonyl)-4-methylphenoxy)methyl)pyrrolidine-1-carboxylate (225A-1)

To a solution of methyl 5-hydroxy-2-methylbenzoate (300 mg, 1.81 mmol, 1.0 eq), (S)-tert-butyl 2-(hydroxymethyl) pyrrolidine-1-carboxylate (363 mg, 1.81 mmol, 1.0 eq) in toluene (15 mL) were added TMAD (933 mg, 5.42 mmol, 3.0 eq) and PPh₃ (1.42 g, 5.42 mmol, 3.0 eq). The mixture was degassed and purged with N₂ three times and stirred at 100° C. for 16 h under a N₂ atmosphere. LCMS indicated that the starting material was completely consumed, and the desired mass was detected. The reaction mixture was allowed to cool to room temperature, poured into H₂O (15 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/0 to 1/5. (S)-tert-Butyl 2-((3-(methoxycarbonyl)-4-methylphenoxy)methyl) pyrrolidine-1-carboxylate (600 mg, 1.72 mmol, 95% yield) was obtained as a yellow oil. M+H⁺=350.1 (LCMS).

### Step 2: (S)-5-((1-(tert-Butoxycarbonyl)pyrrolidin-2-yl)methoxy)-2-methylbenzoic acid (225A-2)

To a solution of(S)-tert-butyl 2-((3-(methoxycarbonyl)-4-methylphenoxy)methyl) pyrrolidine-1-carboxylate (1.20 g, 3.43 mmol, 1.0 eq) in a mixture of MeOH (5.0 mL) and THF (15 mL) was added NaOH (2 M aqueous, 7.0 mL, 4.0 eq). The mixture was stirred at 70° C. for 16 h. LCMS indicated that the starting material was completely consumed, and the desired mass was detected. The reaction mixture was allowed to cool to room temperature, poured into H₂O (30 mL) and extracted with MTBE (15 mL×2). The aqueous layer was acidified to pH 5 using HCl (1 M aqueous). The product was extracted with EtOAc (10 mL×3). The combined organic layers were dried over Na₂SO₄, filtered, and concentrated under vacuum to give the product (S)-5-((1-(tert-butoxycarbonyl)pyrrolidin-2-yl)methoxy)-2-methylbenzoic acid (1.10 g, 96% yield), which was used in the next step without any further purification. M−56+H⁺=280.1 (LCMS).

### Step 3: (S)-tert-Butyl 2-((4-methyl-3-((1-(quinolin-5-yl)cyclopropyl)carbamoyl)phenoxy)methyl)pyrrolidine-1-carboxylate (225A-3)

To a solution of(S)-5-((1-(tert-butoxycarbonyl)pyrrolidin-2-yl)methoxy)-2-methylbenzoic acid (200 mg, 596 μmol, 1.0 eq) and 1-(quinolin-5-yl)cyclopropanamine (110 mg, 596 μmol, 1.0 eq) in DMF (10 mL) were added TEA (181 mg, 1.79 mmol, 249 μL, 3.0 eq), EDCI (286 mg, 1.49 mmol, 2.5 eq) and HOBt (201 mg, 1.49 mmol, 2.5 eq). The mixture was stirred at 20° C. for 16 h. LCMS indicated that the starting material was completely consumed, and the desired mass was detected. The reaction mixture was poured into H₂O (10 mL) and extracted with EtOAc (4.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/0. (S)-tert-Butyl 2-((4-methyl-3-((1-(quinolin-5-yl)cyclopropyl)carbamoyl)phenoxy)methyl) pyrrolidine-1-carboxylate (180 mg, 359 μmol, 60% yield) was obtained as a yellow oil. M+H⁺=502.2 (LCMS).

### Step 4: (S)-2-Methyl-5-(pyrrolidin-2-ylmethoxy)-N-(1-(quinolin-5-yl)cyclopropyl)benzamide (Compound 380)

To a stirred solution of(S)-tert-butyl 2-((4-methyl-3-((1-(quinolin-5-yl)cyclopropyl)carbamoyl)phenoxy)methyl) pyrrolidine-1-carboxylate (180 mg, 359 μmol, 1.0 eq) in EtOAc (2.0 mL) was added HCl/EtOAc (4 M, 10.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna (80×30 mm, 3 μm); flow rate: 25 mL/min; gradient: 1%-20% B over 8 min; mobile phase A: 0.04% aqueous HCl, mobile phase B: acetonitrile). (S)-2-Methyl-5-(pyrrolidin-2-ylmethoxy)-N-(1-(quinolin-5-yl)cyclopropyl)benzamide (27.2 mg, 62.1 μmol, 17% yield, HCl salt) was obtained as a white solid. M+H⁺=402.1 (LCMS); ¹H NMR (400 MHZ, DMSO-d₆) δ 9.58-9.41 (m, 2H), 9.27 (s, 1H), 9.17 (br d, J=4.3 Hz, 1H), 8.98-8.84 (m, 1H), 8.16 (br d, J=8.5 Hz, 1H), 8.06 (br d, J=6.9 Hz, 1H), 8.00-7.89 (m, 2H), 7.09 (d, J=8.6 Hz, 1H), 6.90 (dd, J=2.6, 8.3 Hz, 1H), 6.71 (d, J=2.6 Hz, 1H), 4.16 (dd, J=3.9, 10.6 Hz, 1H), 4.09-4.01 (m, 1H), 3.88-3.75 (m, 1H), 3.23-3.12 (m, 2H), 2.12-2.02 (m, 1H), 1.95 (s, 4H), 1.92-1.82 (m, 1H), 1.73-1.63 (m, 1H), 1.42 (br s, 2H), 1.31-1.22 (m, 2H).

### Example 226: (S)-2-Methyl-5-((1-methylpyrrolidin-2-yl)methoxy)-N-(1-(quinolin-5-yl)cyclopropyl)benzamide (Compound 379)

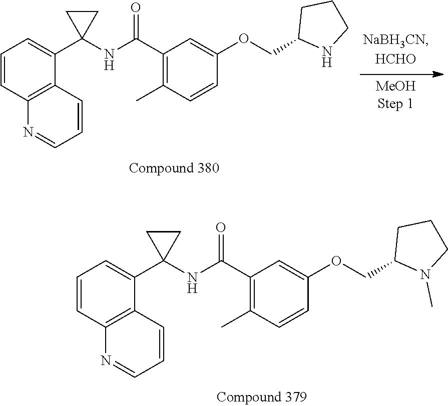 [SMILES: Cc1ccc(OC[C@@H]2CCCN2)cc1C(=O)NC1(c2cccc3ncccc23)CC1 ; Cc1ccc(OC[C@@H]2CCCN2C)cc1C(=O)NC1(c2cccc3ncccc23)CC1]

### Step 1: (S)-2-Methyl-5-((1-methylpyrrolidin-2-yl)methoxy)-N-(1-(quinolin-5-yl)cyclopropyl)benzamide (Compound 379)

To a solution of(S)-2-methyl-5-(pyrrolidin-2-ylmethoxy)-N-(1-(quinolin-5-yl)cyclopropyl)benzamide (130 mg, 324 μmol, 1.0 eq) in MeOH (10 mL) was added TEA (10.0 μL), followed by the addition of formaldehyde (52.6 mg, 648 μmol, 48.2 μL, 37% purity in water, 2.0 eq). The resulting mixture was adjusted to pH 6 with a small amount of AcOH. The mixture was stirred at 20° C. for 30 min, then NaBH₃CN (40.7 mg, 648 μmol, 2.0 eq) was added. The resulting mixture was stirred at 20° C. for another 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Luna (80×30 mm, 3 μm); flow rate: 25 mL/min; gradient: 1%-20% B over 8 min; mobile phase A: 0.04% aqueous HCl, mobile phase B: acetonitrile). (S)-2-Methyl-5-((1-methylpyrrolidin-2-yl)methoxy)-N-(1-(quinolin-5-yl)cyclopropyl)benzamide (26.2 mg, 57.7 μmol, 18% yield, HCl salt) was obtained as a white solid. M+H⁺=416.2 (LCMS); ¹H NMR (400 MHZ, DMSO-d₆) δ 10.76-10.57 (m, 1H), 9.73-9.57 (m, 1H), 9.33 (s, 1H), 9.23 (br s, 1H), 8.23 (br d, J=8.2 Hz, 1H), 8.16-7.95 (m, 3H), 7.09 (d, J=8.3 Hz, 1H), 6.92 (dd, J=2.6, 8.4 Hz, 1H), 6.75 (br s, 1H), 4.31-4.20 (m, 2H), 3.73 (dt, J=3.0, 6.8 Hz, 1H), 3.59-3.48 (m, 1H), 3.15-2.99 (m, 1H), 2.88 (br d, J=4.8 Hz, 3H), 2.29-2.14 (m, 1H), 2.09-1.98 (m, 1H), 1.97-1.86 (m, 4H), 1.83-1.70 (m, 1H), 1.43 (br s, 2H), 1.29 (br s, 2H).

### Example 227: (R)-2-methyl-5-(pyrrolidin-2-ylmethoxy)-N-(1-(quinolin-5-yl)cyclopropyl)benzamide (Compound 371)

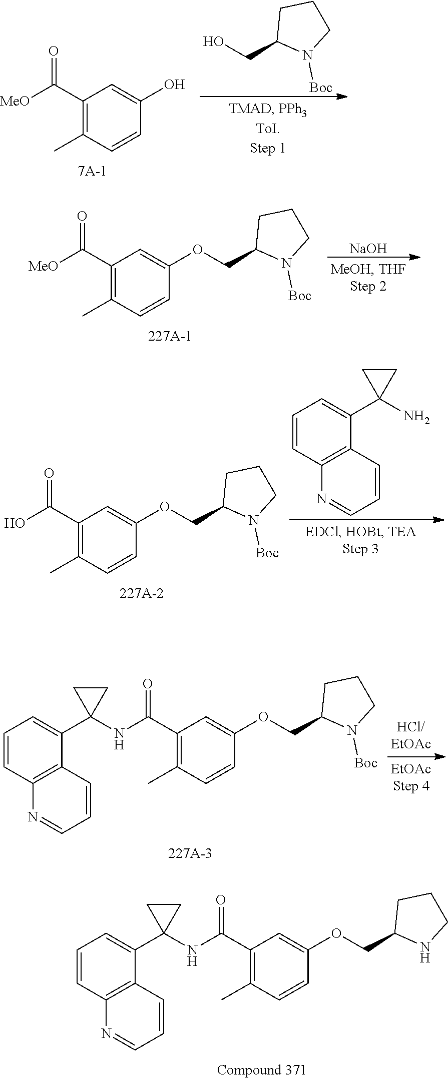

### Step 1: (R)-tert-Butyl 2-((3-(methoxycarbonyl)-4-methylphenoxy)methyl) pyrrolidine-1-carboxylate (227A-1)

To a solution of methyl 5-hydroxy-2-methylbenzoate (300 mg, 1.81 mmol, 1.0 eq) and (R)-tert-butyl 2-(hydroxymethyl) pyrrolidine-1-carboxylate (363 mg, 1.81 mmol, 1.0 eq) in toluene (15 mL) were added TMAD (933 mg, 5.42 mmol, 3.0 eq) and PPh₃ (1.42 g, 5.42 mmol, 3.0 eq). The mixture was degassed and purged with N₂ three times, and then stirred at 100° C. for 16 h under a N₂ atmosphere. LCMS indicated that the starting material was completely consumed, and the desired mass was detected. The reaction mixture was allowed to cool to room temperature, poured into H₂O (10 mL) and extracted with DCM (8.0 mL×3). The combined organic layers were dried over Na₂SO₄, filtered, and concentrated under vacuum to give a residue which was purified by flash silica gel chromatography using a gradient of EtOAc/petroleum ether from 1/100 to 3/5. (R)-tert-Butyl 2-((3-(methoxycarbonyl)-4-methylphenoxy)methyl) pyrrolidine-1-carboxylate (590 mg, 94% yield) was obtained as a white solid. M+H⁺=350.1 (LCMS).

### Step 2: (R)-5-((1-(tert-Butoxycarbonyl)pyrrolidin-2-yl)methoxy)-2-methylbenzoic acid (227A-2)

To a solution of (R)-tert-butyl 2-((3-(methoxycarbonyl)-4-methylphenoxy)methyl) pyrrolidine-1-carboxylate (1.18 g, 3.38 mmol, 1.0 eq) in a mixture of MeOH (47 mL) and THF (24 mL) was added NaOH (2 M aqueous, 7.0 mL, 4.0 eq). The mixture was stirred at 60° C. for 16 h. LCMS indicated that the starting material was completely consumed, and the desired mass was detected. The reaction mixture was poured into H₂O (10 mL) and extracted with EtOAc (10 mL×2). The aqueous layer was acidified to pH 7 using HCl (1 M aqueous). The combined organic layers were dried over Na₂SO₄, filtered, and concentrated under vacuum to give the product (R)-5-((1-(tert-butoxycarbonyl)pyrrolidin-2-yl)methoxy)-2-methylbenzoic acid (1.00 g, 88% yield) as a white solid, which was used in the next step without any further purification.

### Step 3: (R)-tert-Butyl 2-((4-methyl-3-((1-(quinolin-5-yl)cyclopropyl)carbamoyl)phenoxy)methyl)pyrrolidine-1-carboxylate (227A-3)

To a solution of (R)-5-((1-(tert-butoxycarbonyl)pyrrolidin-2-yl)methoxy)-2-methylbenzoic acid (200 mg, 596 μmol, 1.0 eq) and 1-(quinolin-5-yl)cyclopropanamine (109 mg, 596 μmol, 1.0 eq) in DMF (10 mL) were added TEA (121 mg, 1.19 mmol, 166 μL, 2.0 eq), EDCI (171 mg, 894 μmol, 1.5 eq) and HOBt (121 mg, 894 μmol, 1.5 eq). The mixture was stirred at 20° C. for 16 h. LCMS indicated that the starting material was completely consumed, and the desired mass was detected. The reaction mixture was poured into H₂O (6.0 mL) and extracted with EtOAc (4.0 mL×3). The combined organic layers were dried over Na₂SO₄, filtered, and concentrated under vacuum to give a residue which was purified by flash silica gel chromatography using a gradient of EtOAc/petroleum ether from 1/100 to 4/5. (R)-tert-Butyl 2-((4-methyl-3-((1-(quinolin-5-yl)cyclopropyl)carbamoyl)phenoxy)methyl) pyrrolidine-1-carboxylate (200 mg, 399 μmol, 67% yield) was obtained as a yellow gum. M+H⁺=502.3 (LCMS); ¹H NMR (400 MHZ, DMSO-d₆) δ 9.13-9.10 (m, 1H), 9.10-9.06 (m, 1H), 8.92-8.89 (m, 1H), 7.95 (s, 1H), 7.89-7.85 (m, 1H), 7.74-7.67 (m, 1H), 7.60-7.55 (m, 1H), 7.05-7.00 (m, 1H), 6.89-6.84 (m, 1H), 6.69-6.57 (m, 1H), 3.99-3.91 (m, 2H), 3.82-3.73 (m, 1H), 3.27-3.21 (m, 2H), 1.95-1.78 (m, 7H), 1.45-1.31 (m, 9H), 1.30 (br s, 2H), 1.22-1.19 (m, 2H).

### Step 4: (R)-2-Methyl-5-(pyrrolidin-2-ylmethoxy)-N-(1-(quinolin-5-yl)cyclopropyl)benzamide (Compound 371)

To a stirred solution of (R)-tert-butyl 2-((4-methyl-3-((1-(quinolin-5-yl)cyclopropyl) carbamoyl)phenoxy)methyl) pyrrolidine-1-carboxylate (200 mg, 399 μmol, 1.0 eq) in EtOAc (5.0 mL) was added HCl/EtOAc (4 M, 10.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna (80×30 mm, 3 μm); flow rate: 25 mL/min; gradient: 1%-25% B over 8 min; mobile phase A: 0.04% aqueous HCl, mobile phase B: acetonitrile). (R)-2-Methyl-5-(pyrrolidin-2-ylmethoxy)-N-(1-(quinolin-5-yl)cyclopropyl)benzamide (34.4 mg, 78.6 μmol, 20% yield, HCl salt) was obtained as a white solid. M+H⁺=402.1 (LCMS); ¹H NMR (400 MHZ, DMSO-d₆) δ 9.92-9.72 (m, 2H), 9.40 (s, 1H), 9.30 (d, J=4.1 Hz, 1H), 9.24-9.12 (m, 1H), 8.35 (s, 1H), 8.16 (s, 3H), 7.07 (s, 1H), 6.95-6.86 (m, 1H), 6.75 (d, J=2.6 Hz, 1H), 4.20-4.08 (m, 2H), 3.86-3.72 (m, 1H), 3.26-3.09 (m, 2H), 2.16-2.02 (m, 1H), 2.00-1.93 (m, 4H), 1.91-1.83 (m, 1H), 1.73-1.62 (m, 1H), 1.46 (s, 2H), 1.34-1.27 (m, 2H).

### Example 228: (R)-2-Methyl-5-((1-methylpyrrolidin-2-yl)methoxy)-N-(1-(quinolin-5-yl)cyclopropyl)benzamide (Compound 382)

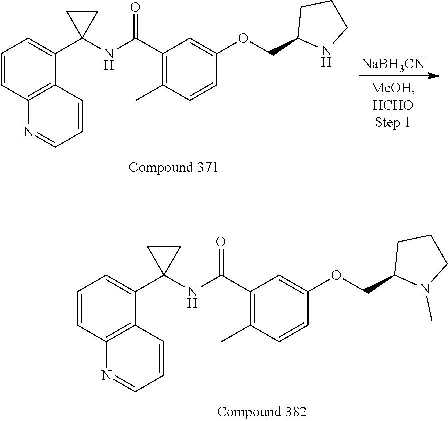

### Step 1: (R)-2-Methyl-5-((1-methylpyrrolidin-2-yl)methoxy)-N-(1-(quinolin-5-yl)cyclopropyl)benzamide (Compound 382)

To a solution of (R)-2-methyl-5-(pyrrolidin-2-ylmethoxy)-N-(1-(quinolin-5-yl)cyclopropyl)benzamide (140 mg, 349 μmol, 1.0 eq) in MeOH (2.0 mL) was added TEA (10.0 μL), followed by the addition of formaldehyde (20.9 mg, 697 μmol, 19.2 μL, 37% purity in water, 2.0 eq). The resulting mixture was adjusted to pH 6 with a small amount of AcOH. The reaction mixture was stirred at 20° C. for 30 min, then NaBH₃CN (43.8 mg, 697 μmol, 2.0 eq) was added. The resulting mixture was stirred at 20° C. for another 16 h. LCMS indicated that the starting material was completely consumed, and the desired mass was detected. The reaction mixture was poured into H₂O (3.0 mL) and extracted with EtOAc (3.0 mL×3). The combined organic layers were dried over Na₂SO₄, filtered, and concentrated under vacuum to give a residue which was purified by preparative HPLC (Phenomenex Luna (80×30 mm, 3 μm); flow rate: 25 mL/min; gradient: 1%-25% B over 8 min; mobile phase A: 0.04% aqueous HCl, mobile phase B: acetonitrile). (R)-2-Methyl-5-((1-methylpyrrolidin-2-yl)methoxy)-N-(1-(quinolin-5-yl)cyclopropyl)benzamide (52.5 mg, 116 μmol, 33% yield, HCl salt) was obtained as a white solid. M+H⁺=416.1 (LCMS); ¹H NMR (400 MHZ, DMSO-d₆) δ 11.06-10.82 (m, 1H), 9.89-9.66 (m, 1H), 9.37 (s, 1H), 9.31-9.23 (m, 1H), 8.37-8.27 (m, 1H), 8.21-7.99 (m, 3H), 7.07 (s, 1H), 6.98-6.85 (m, 1H), 6.76 (s, 1H), 4.38-4.21 (m, 2H), 3.82-3.65 (m, 1H), 3.63-3.45 (m, 1H), 3.16-3.00 (m, 1H), 2.87 (d, J=4.8 Hz, 3H), 2.29-2.15 (m, 1H), 1.95 (m, 5H), 1.83-1.66 (m, 1H), 1.45 (br s, 2H), 1.30 (br s, 2H).

### Example 229: 5-(2-(Dimethylamino)ethoxy)-N-(1-(isoquinolin-5-yl)cyclopropyl)-2-methylbenzamide (Compound 299)

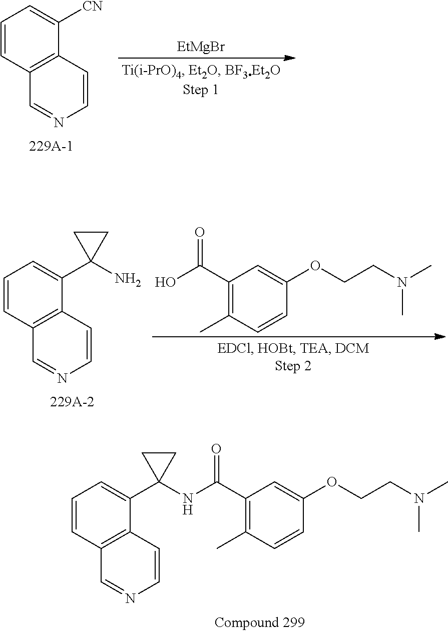 [SMILES: "EtMgBr" ; N#Cc1cccc2cnccc12 ; NC1(c2cccc3cnccc23)CC1 ; Cc1ccc(OCCN(C)C)cc1C(=O)O ; Cc1ccc(OCCN(C)C)cc1C(=O)NC1(c2cccc3cnccc23)CC1]

### Step 1: 1-(Isoquinolin-5-yl)cyclopropanamine (229A-2)

A mixture of isoquinoline-5-carbonitrile (300 mg, 1.95 mmol, 1.0 eq) in anhydrous Et₂O (30 mL) was degassed and purged with N₂ three times. The mixture was stirred at −78° C. To this mixture was added Ti(i-PrO)₄ (608 mg, 2.14 mmol, 632 μL, 1.1 eq) slowly and then EtMgBr (3 M in Et₂O, 1.42 mL, 2.2 eq) was added dropwise to maintain the temperature between-78° C. and −75° C. over 10 min under a N₂ atmosphere. After the addition was complete, the resulting mixture was stirred at the same temperature for 10 min and warmed to 20° C. over 1 h. BF₃·Et₂O (552 mg, 3.89 mmol, 480 μL, 2.0 eq) was added slowly with no obvious temperature change. The mixture was stirred at 20° C. for another 1 h. TLC indicated that the starting material was completely consumed. The reaction mixture was added into a mixture of HCl (1 M aqueous) (20 mL) and MTBE (20 mL) and extracted with MTBE (20 mL×4). The aqueous layer was basified to pH 8 using NaOH (2 M aqueous) and a precipitate was formed. The mixture was filtered through a pad of Celite and the slurry was washed with DCM several times. The combined filtrate was extracted with DCM (10 mL×2). The combined organic layers were dried over Na₂SO₄, filtered, and concentrated under vacuum to give a residue which was purified by flash silica gel chromatography using a gradient of EtOAc/petroleum ether from 1/100 to 1/0. 1-(Isoquinolin-5-yl)cyclopropanamine (100 mg, 543 μmol, 28% yield) was obtained as a yellow oil. M+H⁺=185.1 (LCMS).

### Step 2: 5-(2-(Dimethylamino)ethoxy)-N-(1-(isoquinolin-5-yl)cyclopropyl)-2-methylbenzamide (Compound 299)

To a solution of 5-(2-(dimethylamino)ethoxy)-2-methylbenzoic acid (145 mg, 651 μmol, 1.2 eq) in DCM (5.0 mL) were added TEA (165 mg, 1.63 mmol, 227 μL, 3.0 eq), EDCI (156 mg, 814 μmol, 1.5 eq), HOBt (110 mg, 814 μmol, 1.5 eq) and 1-(isoquinolin-5-yl)cyclopropanamine (100 mg, 543 μmol, 1.0 eq). The mixture was stirred at 25° C. for 16 h. LCMS indicated that the starting material was completely consumed, and the desired mass was detected. The reaction mixture was poured into H₂O (10 mL) at 25° C. and extracted with DCM (5.0 mL×2). The combined organic layers were dried over Na₂SO₄, filtered, and concentrated under vacuum to give a residue which was purified by preparative HPLC (Waters Xbridge Prep OBD C18 (150×40 mm, 10 μm); flow rate: 25 mL/min; gradient: 15%-45% B over 8 min; mobile phase A: 10 mM aqueous NH₄HCO₃, mobile phase B: acetonitrile). 5-(2-(Dimethylamino)ethoxy)-N-(1-(isoquinolin-5-yl)cyclopropyl)-2-methylbenzamide (41.6 mg, 100 μmol, 18% yield) was obtained as a yellow solid. M+H⁺=390.2 (LCMS); ¹H NMR (400 MHz, DMSO-d₆) δ 9.31 (s, 1H), 9.15 (s, 1H), 8.56 (d, J=6.0 Hz, 1H), 8.47 (d, J=6.0 Hz, 1H), 8.06-7.98 (m, 2H), 7.64 (t, J=7.6 Hz, 1H), 7.02 (d, J=8.4 Hz, 1H), 6.89-6.79 (m, 1H), 6.61 (d, J=2.7 Hz, 1H), 3.94 (t, J=5.8 Hz, 2H), 2.56-2.52 (m, 2H), 2.16 (s, 6H), 1.93 (s, 3H), 1.38-1.32 (m, 2H), 1.22-1.14 (m, 2H).

### Example 230: N-(1-(Isoquinolin-5-yl)cyclopropyl)-2-methyl-5-(2-(methylamino)ethoxy)benzamide (Compound 375)

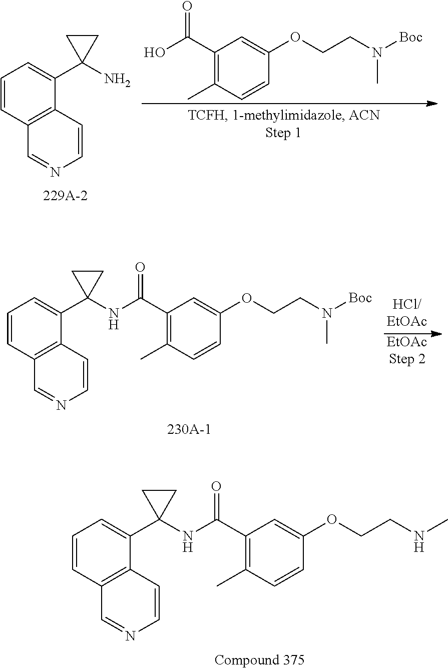 [SMILES: NC1(c2cccc3cnccc23)CC1 ; Cc1ccc(OCCN(C)C(=O)OC(C)(C)C)cc1C(=O)O ; Cc1ccc(OCCN(C)C(=O)OC(C)(C)C)cc1C(=O)NC1(c2cccc3cnccc23)CC1 ; "EtOAcStep 2" ; CNCCOc1ccc(C)c(C(=O)NC2(c3cccc4cnccc34)CC2)c1]

### Step 1: tert-Butyl(2-(3-((1-(isoquinolin-5-yl)cyclopropyl)carbamoyl)-4-methylphenoxy)ethyl)(methyl)carbamate (230A-1)

To a solution of 1-(isoquinolin-5-yl)cyclopropanamine (50.0 mg, 231 μmol, 1.0 eq) and 5-(2-((tert-butoxycarbonyl)(methyl)amino)ethoxy)-2-methylbenzoic acid (71.4 mg, 231 μmol, 1.0 eq) in acetonitrile (3.0 mL) were added TCFH (77.7 mg, 277 μmol, 1.2 eq) and 1-methylimidazole (66.3 mg, 807 μmol, 64.4 μL, 3.5 eq). The mixture was stirred at 20° C. for 18 h. LCMS indicated that the starting material was completely consumed, and the desired mass was detected. The reaction mixture was poured into H₂O (5.0 mL) and extracted with DCM (5.0 mL×5). The combined organic layers were dried over Na₂SO₄, filtered, and concentrated under vacuum to give a residue which was purified by preparative TLC (pure EtOAc, R_{f}=0.3). tert-Butyl(2-(3-((1-(isoquinolin-5-yl)cyclopropyl)carbamoyl)-4-methylphenoxy)ethyl)(methyl)carbamate (30.0 mg, 63.1 μmol, 27% yield) was obtained as a white solid. M+H⁺=476.2 (LCMS).

### Step 2: N-(1-(Isoquinolin-5-yl)cyclopropyl)-2-methyl-5-(2-(methylamino)ethoxy)benzamide (Compound 375)

To a solution of tert-butyl(2-(3-((1-(isoquinolin-5-yl)cyclopropyl)carbamoyl)-4-methyl phenoxy)ethyl)(methyl)carbamate (30.0 mg, 631 μmol, 1.0 eq) in EtOAc (3.0 mL) was added HCl/EtOAc (4 M, 189 μL). The mixture was stirred at 20° C. for 1 h. LCMS indicated that the starting material was completely consumed, and the desired mass was detected. The mixture was concentrated under vacuum to give a residue which was purified by preparative HPLC (Phenomenex Luna C18 column (80×30 mm, 3 μm); flow rate: 25 mL/min; gradient: 1%-25% B over 8 min; mobile phase A: 0.04% aqueous HCl, mobile phase B: acetonitrile). N-(1-(Isoquinolin-5-yl)cyclopropyl)-2-methyl-5-(2-(methylamino)ethoxy)benzamide (19.6 mg, 47.3 μmol, 75% yield, HCl salt) was obtained as a white solid. M+H⁺=376.1 (LCMS); 1H NMR (400 MHZ, CD₃OD) δ 9.83 (s, 1H), 9.22 (d, J=6.8 Hz, 1H), 8.69 (d, J=6.9 Hz, 1H), 8.63 (dd, J=0.9, 7.3 Hz, 1H), 8.49 (d, J=8.3 Hz, 1H), 8.10-8.03 (m, 1H), 7.11 (d, J=8.5 Hz, 1H), 6.95 (dd, J=2.8, 8.5 Hz, 1H), 6.82 (d, J=2.6 Hz, 1H), 4.24-4.18 (m, 2H), 3.44-3.38 (m, 2H), 2.76 (s, 3H), 2.00 (s, 3H), 1.63-1.57 (m, 2H), 1.46-1.39 (m, 2H).

### Example 231: N-(1-(Isoquinolin-5-yl)cyclopropyl)-2-methyl-5-((1-methylazetidin-2-yl)methoxy)benzamide (Compound 402)

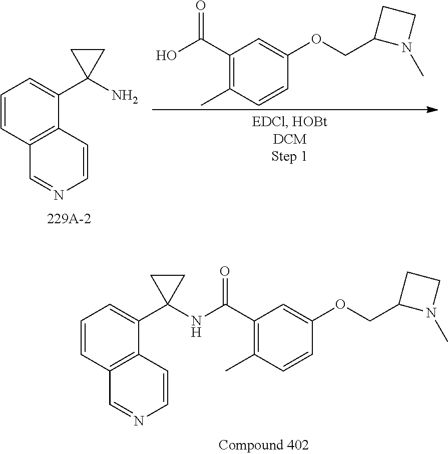 [SMILES: NC1(c2cccc3cnccc23)CC1 ; Cc1ccc(OCC2CCN2C)cc1C(=O)O ; Cc1ccc(OCC2CCN2C)cc1C(=O)NC1(c2cccc3cnccc23)CC1]

### Step 1: N-(1-(Isoquinolin-5-yl)cyclopropyl)-2-methyl-5-((1-methylazetidin-2-yl)methoxy)benzamide (Compound 402)

To a solution of 1-(isoquinolin-5-yl)cyclopropanamine (40 mg, 217 μmol, 1.0 eq) and 2-methyl-5-((1-methylazetidin-2-yl)methoxy)benzoic acid (91.9 mg, 391 μmol, 1.8 eq) in DCM (2.0 mL) were added TEA (65.9 mg, 651 mmol, 90.6 μL, 3.0 eq), EDCI (104 mg, 543 μmol, 2.5 eq) and HOBt (73.3 mg, 543 mmol, 2.5 eq). The mixture was stirred at 20° C. for 3 h. LCMS indicated that the starting material was completely consumed, and the desired mass was detected. The reaction mixture was poured into H₂O (3.0 mL) and extracted with DCM (3.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 1%-30% B over 8 min; mobile phase A: 0.2% aqueous FA, mobile phase B: acetonitrile). N-(1-(Isoquinolin-5-yl)cyclopropyl)-2-methyl-5-((1-methylazetidin-2-yl)methoxy)benzamide (15.1 mg, 33.1 μmol, 15% yield, FA salt) was obtained as a white solid. M+H⁺=402.1 (LCMS); ¹H NMR (400 MHZ, DMSO-d₆) δ 9.32 (s, 1H), 9.16 (s, 1H), 8.57 (d, J=6.0 Hz, 1H), 8.48 (d, J=6.0 Hz, 1H), 8.19 (s, 1H), 8.03 (d, J=7.6 Hz, 2H), 7.65 (t, J=7.7 Hz, 1H), 7.03 (d, J=8.4 Hz, 1H), 6.84 (dd, J=2.7, 8.3 Hz, 1H), 6.62 (d, J=2.6 Hz, 1H), 3.90 (d, J=4.9 Hz, 2H), 3.49-3.13 (m, 2H), 2.83 (q, J=8.2 Hz, 1H), 2.27 (s, 3H), 2.06-1.95 (m, 1H), 1.94 (s, 3H), 1.92-1.81 (m, 1H), 1.36 (s, 2H), 1.25-1.13 (m, 2H).

### Example 232: 5-(2-Aminoethoxy)-2-methyl-N-(1-(naphthalen-1-yl)cyclopropyl)benzamide (Compound 333)

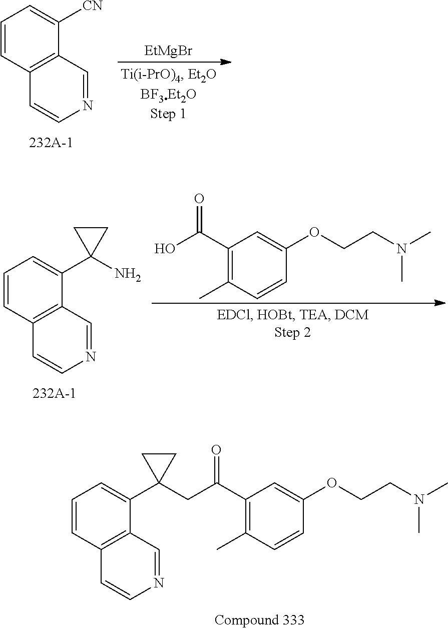 [SMILES: "EtMgBr" ; N#Cc1cccc2ccncc12 ; NC1(c2cccc3ccncc23)CC1 ; Cc1ccc(OCCN(C)C)cc1C(=O)O ; Cc1ccc(OCCN(C)C)cc1C(=O)CC1(c2cccc3ccncc23)CC1]

### Step 1: 1-(Isoquinolin-8-yl)cyclopropanamine (232A-2)

A mixture of isoquinoline-8-carbonitrile (100 mg, 649 μmol, 1.0 eq) in anhydrous Et₂O (7.0 mL) was degassed and purged with N₂ three times. The mixture was stirred at −78° C. To this mixture was added Ti(i-PrO)+ (203 mg, 714 μmol, 211 μL, 1.1 eq) slowly, and then EtMgBr (3 M in Et₂O, 476 mL, 2.2 eq) was added dropwise to maintain the temperature between −78° C. and −75° C. over 5 min under a N₂ atmosphere. After the addition was complete, the resulting mixture was stirred at the same temperature for 10 min and warmed to 20° C. over 1 h. BF₃·Et₂O (185 mg, 1.30 mmol, 160 μL, 2.0 eq) was added slowly with no obvious temperature change. The mixture was stirred at 20° C. for another 1 h. LCMS indicated that 40% starting material still remained and 13% desired compound was detected. The reaction mixture was added into a mixture of HCl (1 M aqueous) (10 mL) and MTBE (10 mL), and extracted with MTBE (10 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (5 mL×4). The combined organic layers were dried over Na₂SO₄, filtered, and concentrated under vacuum to give a residue which was purified by preparative HPLC (C18 column (250×50 mm, 10 μm); flow rate: 60 mL/min; gradient: 20%-40% B over 8 min; mobile phase A: 10 mM aqueous NH₄HCO₃, mobile phase B: acetonitrile). 1-(Isoquinolin-8-yl)cyclopropanamine (15 mg, 81.4 μmol, 12% yield) was obtained as a white solid. M+H⁺=185.1 (LCMS).

### Step 2: 5-(2-(Dimethylamino)ethoxy)-N-(1-(isoquinolin-8-yl)cyclopropyl)-2-methylbenzamide (Compound 333)

To a solution of 1-(isoquinolin-8-yl)cyclopropanamine (30.0 mg, 163 μmol, 1.0 eq) and 5-(2-(dimethylamino)ethoxy)-2-methylbenzoic acid (72.7 mg, 326 μmol, 2.0 eq) in DCM (1.0 mL) were added TEA (49.4 mg, 488 μmol, 67.9 μL, 3.0 eq), EDCI (37.5 mg, 195 μmol, 1.2 eq) and HOBt (26.4 mg, 195 μmol, 1.2 eq). The mixture was stirred at 25° C. for 3 h. LCMS indicated that the starting material was completely consumed, and the desired mass was detected. The reaction mixture was poured into H₂O (4.0 mL) and extracted with DCM (2.0 mL×3). The combined organic layers were dried over Na₂SO₄, filtered, and concentrated under vacuum to give a residue which was purified by preparative HPLC (Phenomenex C18 column (75×30 mm, 3 μm); flow rate: 25 mL/min; gradient: 10%-50% B over 8 min; mobile phase A: 10 mM aqueous NH₄HCO₃, mobile phase B: acetonitrile). 5-(2-(Dimethylamino)ethoxy)-N-(1-(isoquinolin-8-yl)cyclo propyl)-2-methylbenzamide (3.20 mg, 8.22 μmol, 5% yield) was obtained as a white solid. M+H⁺=390.1 (LCMS); ¹H NMR (400 MHZ, DMSO-d₆) δ 10.04 (s, 1H), 9.24 (s, 1H), 8.53 (d, J=5.5 Hz, 1H), 8.04-7.79 (m, 3H), 7.77-7.59 (m, 1H), 7.03 (d, J=8.3 Hz, 1H), 6.84 (dd, J=2.6, 8.7 Hz, 1H), 6.63 (d, J=2.6 Hz, 1H), 3.95 (s, 2H), 2.60-2.54 (m, 2H), 2.16 (s, 6H), 1.93 (s, 3H), 1.39 (br s, 2H), 1.26 (br s, 2H).

### Example 233: 5-(2-(Dimethylamino)ethoxy)-2-methyl-N-(1-(quinolin-8-yl)cyclopropyl)benzamide (Compound 388)

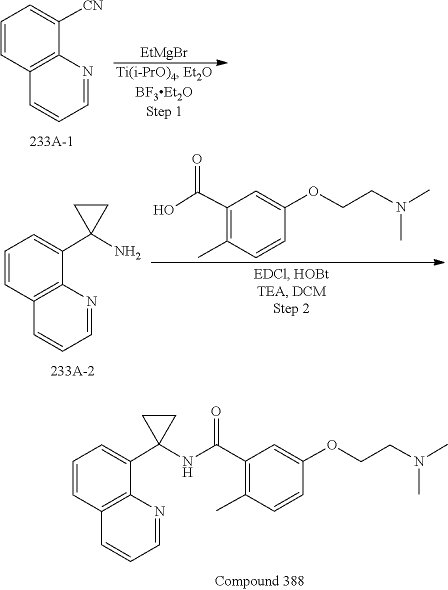 [SMILES: N#Cc1cccc2cccnc12 ; NC1(c2cccc3cccnc23)CC1 ; Cc1ccc(OCCN(C)C)cc1C(=O)O ; Cc1ccc(OCCN(C)C)cc1C(=O)NC1(c2cccc3cccnc23)CC1]

### Step 1: 1-(Quinolin-8-yl)cyclopropanamine (233A-2)

To a mixture of quinoline-8-carbonitrile (300 mg, 1.95 mmol, 1.0 eq) in anhydrous Et₂O (20 mL) was degassed and purged with N₂ three times. The mixture was stirred at −78° C. To this mixture was added Ti(i-PrO)₄ (608 mg, 2.14 mmol, 630 μL, 1.1 eq) slowly, and then EtMgBr (3 M in Et₂O, 1.43 mL, 2.2 eq) was added dropwise to maintain the temperature between-78° C. and −75° C. over 5 min under a N₂ atmosphere. After the addition was complete, the resulting mixture was stirred at the same temperature for 10 min and warmed to 20° C. over 1 h. BF₃·Et₂O (552 mg, 3.89 mmol, 480 μL, 2.0 eq) was added slowly with no obvious temperature change. The mixture was stirred at 20° C. for another 1 h. TLC indicated that the starting material was completely consumed, and a main spot was detected. The reaction mixture was added into a mixture of HCl (1 M aqueous) (20 mL) and MTBE (20 mL), and extracted with MTBE (20 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (20 ml×2). The combined organic layers were dried over Na₂SO₄, filtered, and concentrated under vacuum to give a residue which was purified by flash silica gel chromatography using a gradient of EtOAc/petroleum ether from 0/1 to 1/1. 1-(Quinolin-8-yl)cyclopropanamine (60.0 mg, 330 μmol, 17% yield) was obtained as a yellow oil. M+H⁺=185.1 (LCMS).

### Step 2: 5-(2-(Dimethylamino)ethoxy)-2-methyl-N-(1-(quinolin-8-yl)cyclopropyl)benzamide (Compound 388)

To a solution of 1-(quinolin-8-yl)cyclopropanamine (40.0 mg, 217 μmol, 1.0 eq) and 5-(2-(dimethylamino)ethoxy)-2-methylbenzoic acid (48.5 mg, 217 μmol, 1.0 eq) in DCM (2.0 mL) were added TEA (44.0 mg, 430 μmol, 60.4 μL, 2.0 eq), EDCI (62.4 mg, 330 μmol, 1.5 eq) and HOBt (44.0 mg, 330 μmol, 1.5 eq). The mixture was stirred at 20° C. for 1 h. LCMS indicated that the starting material was completely consumed, and the desired mass was detected. The reaction mixture was poured into H₂O (2.0 mL) and extracted with DCM (2.0 mL×2). The combined organic layers were dried over Na₂SO₄, filtered, and concentrated under vacuum to give a residue which was purified by preparative HPLC (Phenomenex Gemini C18 column (75× 30 mm, 3 μm); flow rate: 25 mL/min; gradient: 25%-45% B over 8 min; mobile phase A: 10 mM NH₄HCO₃, mobile phase B: acetonitrile). 5-(2-(Dimethylamino)ethoxy)-2-methyl-N-(1-(quinolin-8-yl)cyclopropyl)benzamide (15.0 mg, 38.1 μmol, 18% yield) was obtained as a white solid. M+H⁺=390.2 (LCMS); ¹H NMR (400 MHZ, DMSO-d₆) δ 8.90-9.03 (m, 1H), 8.68 (s, 1H), 8.27-8.44 (m, 1H), 7.81-7.99 (m, 2H), 7.47-7.64 (m, 2H), 7.00 (br d, J=8.38 Hz, 1H), 6.75-6.86 (m, 1H), 6.61-6.71 (m, 1H), 3.95 (br t, J=5.50 Hz, 2H), 2.54-2.63 (m, 2H), 2.19 (br s, 6H), 1.97 (s, 3H), 1.33 (br d, J=8.63 Hz, 4H).

### Example 234: 5-(2-Aminoethoxy)-N-(1-(3-methoxynaphthalen-1-yl)cyclopropyl)-2-methylbenzamide (Compound 363)

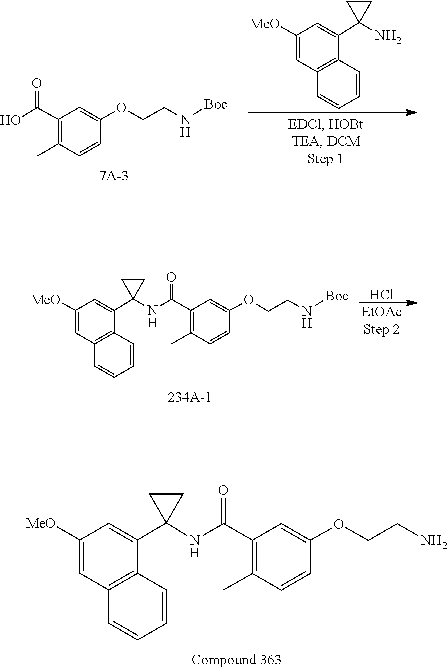 [SMILES: Cc1ccc(OCCNC(=O)OC(C)(C)C)cc1C(=O)O ; COc1cc(C2(N)CC2)c2ccccc2c1 ; COc1cc(C2(NC(=O)c3cc(OCCNC(=O)OC(C)(C)C)ccc3C)CC2)c2ccccc2c1 ; COc1cc(C2(NC(=O)c3cc(OCCN)ccc3C)CC2)c2ccccc2c1]

### Step 1: tert-Butyl(2-(3-((1-(3-methoxynaphthalen-1-yl)cyclopropyl)carbamoyl)-4-methylphenoxy)ethyl)carbamate (234A-1)

To a solution of 5-(2-((tert-butoxycarbonyl)amino)ethoxy)-2-methylbenzoic acid (166 mg, 563 μmol, 1.2 eq) and 1-(3-methoxynaphthalen-1-yl)cyclopropanamine (100 mg, 469 μmol, 1.0 eq) in DCM (1.0 mL) were added TEA (142 mg, 1.41 mmol, 196 μL, 3.0 eq), EDCI (135 mg, 703 μmol, 1.5 eq) and HOBt (95.0 mg, 703 μmol, 1.5 eq). The mixture was stirred at 20° C. for 16 h. LCMS indicated that the starting material was completely consumed, and the desired mass was detected. The reaction mixture was poured into H₂O (10 mL) and extracted with DCM (10 mL×2). The combined organic layers were dried over Na₂SO₄, filtered, and concentrated under vacuum to give a residue which was purified by preparative TLC (EtOAc/petroleum ether=2/1, R_{f}=0.4) to give tert-butyl(2-(3-((1-(3-methoxynaphthalen-1-yl)cyclopropyl)carbamoyl)-4-methyl phenoxy)ethyl)carbamate (200 mg, 408 μmol, 87% yield) as a yellow oil. M+H⁺=491.2 (LCMS).

### Step 2: 5-(2-Aminoethoxy)-N-(1-(3-methoxynaphthalen-1-yl)cyclopropyl)-2-methylbenzamide (Compound 363)

To a stirred solution of tert-butyl(2-(3-((1-(3-methoxynaphthalen-1-yl)cyclopropyl) carbamoyl)-4-methyl phenoxy)ethyl)carbamate (200 mg, 408 μmol, 1.0 eq) in EtOAc (1.0 mL) was added HCl/EtOAc (4 M, 5.0 mL). The mixture was stirred at 20° C. for 4 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 5%-55% B over 8 min; mobile phase A: 0.04% aqueous HCl, mobile phase B: acetonitrile) to give 5-(2-aminoethoxy)-N-(1-(3-methoxynaphthalen-1-yl)cyclopropyl)-2-methylbenzamide (150 mg, 383 μmol, 94% yield, HCl salt) as a white solid. M+H⁺=391.1 (LCMS); ¹H NMR (400 MHz, DMSO-d₆) δ 9.12 (s, 1H), 8.53 (d, J=8.3 Hz, 1H), 8.04 (br s, 3H), 7.83 (d, J=7.9 Hz, 1H), 7.49-7.37 (m, 3H), 7.24 (d, J=2.5 Hz, 1H), 7.08 (d, J=8.4 Hz, 1H), 6.88 (dd, J=2.6, 8.4 Hz, 1H), 6.66 (d, J=2.6 Hz, 1H), 4.08 (t, J=5.1 Hz, 2H), 3.87 (s, 3H), 3.19-3.08 (m, 2H), 1.98 (s, 3H), 1.33 (br s, 2H), 1.20-1.11 (m, 2H).

### Example 235: 5-(2-(Dimethylamino)ethoxy)-N-(1-(3-methoxynaphthalen-1-yl)cyclopropyl)-2-methylbenzamide (Compound 320)

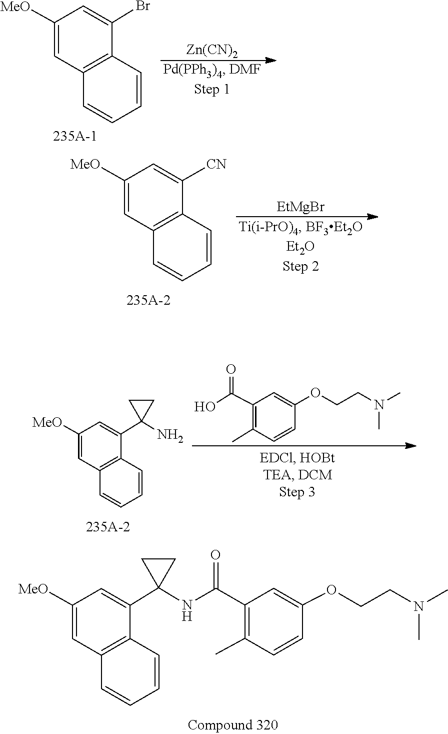

### Step 1:3-Methoxy-1-naphthonitrile (235A-2)

To a solution of 1-bromo-3-methoxynaphthalene (2.00 g, 8.44 mmol, 1.0 eq) in DMF (40 mL) were added Zn(CN)₂ (1.98 g, 16.9 mmol, 1.07 mL, 2.0 eq) and Pd(PPh₃)₄ (975 mg, 844 μmol, 0.1 eq). The mixture was stirred at 120° C. for 6 h under a N₂ atmosphere. TLC indicated that the starting material was completely consumed, and a main spot was detected. The reaction mixture was allowed to cool to room temperature, poured into H₂O (60 mL) and extracted with EtOAc (30 mL×3). The combined organic layers were dried over Na₂SO₄, filtered, and concentrated under vacuum to give a residue which was purified by flash silica gel chromatography using a gradient of EtOAc/petroleum ether from 1/100 to 1/5. 3-Methoxy-1-naphthonitrile (1.50 g, 97% yield) was obtained as a white solid. ¹H NMR (400 MHZ, DMSO-d₆) δ 8.03-7.97 (m, 2H), 7.88 (d, J=2.6 Hz, 1H), 7.76 (d, J=2.5 Hz, 1H), 7.68-7.58 (m, 2H), 3.93 (s, 3H).

### Step 2: 1-(3-Methoxynaphthalen-1-yl)cyclopropanamine (235A-3)

A mixture of 3-methoxynaphthalene-1-carbonitrile (1.40 g, 7.64 mmol, 1.0 eq) in anhydrous Et₂O (100 mL) was degassed and purged with N₂ three times. The mixture was stirred at −78° C. To this mixture was added Ti(i-PrO)₄ (2.39 g, 8.41 mmol, 2.48 mL, 1.1 eq) slowly, and then EtMgBr (3 M, in Et₂O, 5.60 mL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. BF₃·Et₂O (2.17 g, 15.3 mmol, 1.89 mL, 2.0 eq) was added slowly with no obvious temperature change. The mixture was stirred at 20° C. for another 1 h. TLC indicated that the starting material was completely consumed, and a main spot was detected. The reaction mixture was added into a mixture of HCl (1 M aqueous) (50 mL) and MTBE (50 mL), and extracted with MTBE (50 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (50 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. 3-Methoxy-1-naphthonitrile (500 mg, 2.34 mmol, 31% yield) was obtained as a brown solid. M+H⁺=214.2 (LCMS); ¹H NMR (400 MHz, CDCl₃) δ 8.35 (d, J=8.0 Hz, 1H), 7.79 (d, J=8.0 Hz, 1H), 7.52-7.39 (m, 2H), 7.20 (d, J=2.3 Hz, 1H), 7.06 (d, J=1.9 Hz, 1H), 3.93 (s, 3H), 2.04 (br s, 2H), 1.23-1.16 (m, 2H), 1.06-0.99 (m, 2H).

### Step 3: 5-(2-(Dimethylamino)ethoxy)-N-(1-(3-methoxynaphthalen-1-yl)cyclopropyl)-2-methylbenzamide (Compound 320)

To a solution of 1-(3-methoxynaphthalen-1-yl)cyclopropanamine (100 mg, 469 μmol, 1.0 eq) and 5-(2-(dimethylamino)ethoxy)-2-methylbenzoic acid (105 mg, 469 μmol, 1.0 eq) in DCM (6.0 mL) were added TEA (142 mg, 1.41 mmol, 196 μL, 3.0 eq), EDCI (225 mg, 1.17 mmol, 2.5 eq) and HOBt (158 mg, 1.17 mmol, 2.5 eq). The mixture was stirred at 20° C. for 16 h. LCMS indicated that the starting material was completely consumed, and the desired mass was detected. The reaction mixture was poured into H₂O (10 mL) and extracted with DCM (10 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 15%-50% B over 8 min; mobile phase A: 0.2% aqueous FA, mobile phase B: acetonitrile) to give 5-(2-(dimethylamino)ethoxy)-N-(1-(3-methoxynaphthalen-1-yl)cyclopropyl)-2-methylbenzamide (57.5 mg, 124 μmol, 26% yield, FA salt) as a white solid. M+H⁺=419.1 (LCMS); ¹H NMR (400 MHZ, DMSO-d₆) δ 9.06 (s, 1H), 8.54 (d, J=8.3 Hz, 1H), 8.19 (s, 1H), 7.83 (d, J=7.9 Hz, 1H), 7.49-7.35 (m, 3H), 7.23 (d, J=2.5 Hz, 1H), 7.03 (d, J=8.5 Hz, 1H), 6.83 (dd, J=2.8, 8.4 Hz, 1H), 6.61 (d, J=2.6 Hz, 1H), 3.95 (t, J=5.8 Hz, 2H), 3.87 (s, 3H), 2.58 (t, J=5.8 Hz, 2H), 2.19 (s, 6H), 1.97 (s, 3H), 1.33 (br s, 2H), 1.19-1.12 (m, 2H).

### Example 236: 5-(2-(Dimethylamino)ethoxy)-N-(1-(3-hydroxynaphthalen-1-yl)cyclopropyl)-2-methylbenzamide (Compound 345)

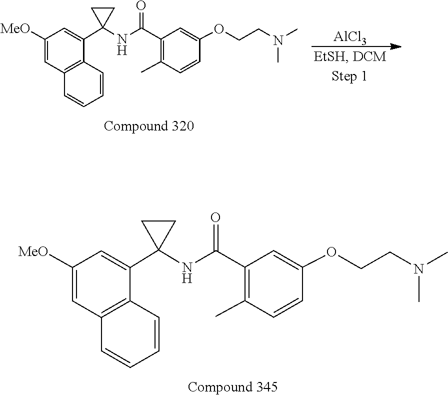 [SMILES: COc1cc(C2(NC(=O)c3cc(OCCN(C)C)ccc3C)CC2)c2ccccc2c1 ; COc1cc(C2(NC(=O)c3cc(OCCN(C)C)ccc3C)CC2)c2ccccc2c1]

### Step 1: 5-(2-(Dimethylamino)ethoxy)-N-(1-(3-hydroxynaphthalen-1-yl)cyclopropyl)2-methylbenzamide (Compound 345)

To a solution of EtSH (1.05 g, 16.9 mmol, 1.25 mL, 141 eq) in DCM (5.0 mL) was added AlCl₃ (95.6 mg, 717 μmol, 39.2 μL, 6.0 eq) at 0° C. To the resulting mixture was added 5-[2-(dimethylamino)ethoxy]-N-[1-(3-methoxy-1-naphthyl)cyclopropyl]-2-methyl-benzamide (50.0 mg, 119 μmol, 1.0 eq) in DCM (0.5 mL) at 0° C. The resulting mixture was stirred at 0° C. for 10 min, then warmed to 20° C. and stirred at the same temperature for 2 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 5%-40% B over 8 min; mobile phase A: 0.2% aqueous FA, mobile phase B: acetonitrile). 5-(2-(Dimethylamino)ethoxy)-N-(1-(3-hydroxynaphthalen-1-yl)cyclopropyl)-2-methylbenzamide (8.70 mg, 19.2 μmol, 16% yield, FA salt) was obtained as a white solid. M+H⁺=405.1 (LCMS); ¹H NMR (400 MHZ, DMSO-d₆) δ 9.04 (s, 1H), 8.49 (d, J=8.4 Hz, 1H), 8.18 (s, 1H), 7.68 (d, J=8.0 Hz, 1H), 7.42 (d, J=2.4 Hz, 1H), 7.38 (t, J=7.4 Hz, 1H), 7.33-7.27 (m, 1H), 7.08-7.00 (m, 2H), 6.83 (dd, J=2.6, 8.3 Hz, 1H), 6.62 (d, J=2.5 Hz, 1H), 3.95 (br t, J=5.5 Hz, 2H), 2.58 (br t, J=5.5 Hz, 2H), 2.19 (s, 6H), 1.98 (s, 3H), 1.32 (br s, 2H), 1.13 (br s, 2H).

### Example 237: N-(1-(3-Methoxynaphthalen-1-yl)cyclopropyl)-2-methyl-5-(2-(methylamino)ethoxy)benzamide (Compound 347)

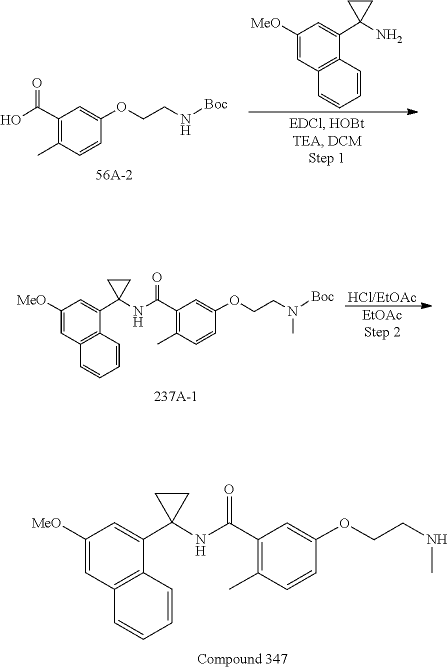 [SMILES: Cc1ccc(OCCNC(=O)OC(C)(C)C)cc1C(=O)O ; COc1cc(C2(N)CC2)c2ccccc2c1 ; COc1cc(C2(NC(=O)c3cc(OCCN(C)C(=O)OC(C)(C)C)ccc3C)CC2)c2ccccc2c1 ; CNCCOc1ccc(C)c(C(=O)NC2(c3cc(OC)cc4ccccc34)CC2)c1]

### Step 1: tert-Butyl(2-(3-((1-(3-methoxynaphthalen-1-yl)cyclopropyl)carbamoyl)-4-methylphenoxy)ethyl)(methyl)carbamate (237A-1)

To a solution of 5-(2-((tert-butoxycarbonyl)(methyl)amino)ethoxy)-2-methylbenzoic acid (100 mg, 323 μmol, 1.0 eq) and 1-(3-methoxynaphthalen-1-yl)cyclopropanamine (68.9 mg, 323 μmol, 1.0 eq) in DCM (10 mL) were added TEA (98.1 mg, 970 μmol, 135 μL, 3.0 eq), EDCI (155 mg, 808 μmol, 2.5 eq) and HOBt (109 mg, 808 μmol, 2.5 eq). The mixture was stirred at 20° C. for 16 h. LCMS indicated that the starting material was completely consumed, and the desired mass was detected. The reaction mixture was poured into H₂O (10 mL) and extracted with EtOAc (4.0 mL×3). The combined organic layers were dried over Na₂SO₄, filtered, and concentrated under vacuum to give a residue which was purified by was purified by preparative TLC (EtOAc/petroleum ether=1/1, R_{f}=0.5). tert-Butyl(2-(3-((1-(3-methoxynaphthalen-1-yl)cyclopropyl)carbamoyl)-4-methylphenoxy)ethyl)(methyl)carbamate (100 mg, 198 μmol, 61% yield) was obtained as a yellow oil. M+H⁺=505.3 (LCMS).

### Step 2: N-(1-(3-Methoxynaphthalen-1-yl)cyclopropyl)-2-methyl-5-(2-(methylamino)ethoxy)benzamide (Compound 347)

To a stirred solution of tert-butyl(2-(3-((1-(3-methoxynaphthalen-1-yl)cyclopropyl) carbamoyl)-4-methylphenoxy)ethyl)(methyl)carbamate (100 mg, 198 μmol, 1.0 eq) in EtOAc (2.0 mL) was added HCl/EtOAc (4 M, 10.0 mL). The mixture was stirred at 20° C. for 2 h. LCMS indicated that the starting material was completely consumed, and the desired mass was detected. The mixture was concentrated under vacuum at 30° C. to give a residue which was purified by preparative HPLC (Phenomenex Luna (80×30 mm, 3 μm); flow rate: 25 mL/min; gradient: 10%-70% B over 8 min; mobile phase A: 0.04% aqueous HCl, mobile phase B: acetonitrile). N-(1-(3-Methoxynaphthalen-1-yl)cyclopropyl)-2-methyl-5-(2-(methylamino)ethoxy)benzamide (48.3 mg, 109 μmol, 55% yield, HCl salt) was obtained as a white solid. M+H⁺=405.1 (LCMS); ¹H NMR (400 MHZ, DMSO-d₆) δ 9.14 (br d, J=1.5 Hz, 1H), 8.87 (br d, J=1.0 Hz, 2H), 8.53 (br d, J=5.4 Hz, 1H), 7.91-7.77 (m, 1H), 7.53-7.33 (m, 3H), 7.24 (br s, 1H), 7.08 (br d, J=5.6 Hz, 1H), 6.97-6.84 (m, 1H), 6.67 (br s, 1H), 4.15 (br s, 2H), 3.87 (br s, 3H), 3.25 (br s, 2H), 2.57 (br s, 3H), 1.98 (br s, 3H), 1.33 (br s, 2H), 1.16 (br s, 2H).

### Example 238: N-(1-(3-methoxynaphthalen-1-yl)cyclopropyl)-2-methyl-5-((1-methylazetidin-2-yl)methoxy)benzamide (Compound 384)

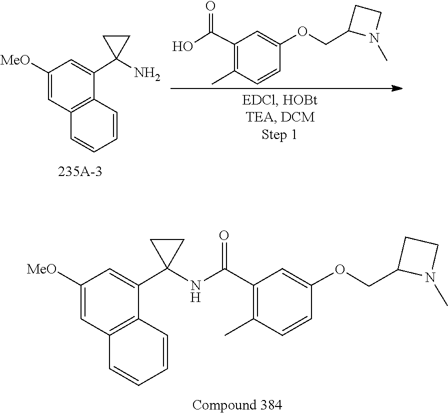 [SMILES: COc1cc(C2(N)CC2)c2ccccc2c1 ; Cc1ccc(OCC2CCN2C)cc1C(=O)O ; COc1cc(C2(NC(=O)c3cc(OCC4CCN4C)ccc3C)CC2)c2ccccc2c1]

### Step 1: N-(1-(3-Methoxynaphthalen-1-yl)cyclopropyl)-2-methyl-5-((1-methylazetidin-2-yl)methoxy)benzamide (Compound 384)

To a solution of 2-methyl-5-((1-methylazetidin-2-yl)methoxy)benzoic acid (80.0 mg, 340 μmol, 1.0 eq) and 1-(3-methoxynaphthalen-1-yl)cyclopropanamine (72.5 mg, 340 μmol, 1.0 eq) in DCM (6.0 mL) were added TEA (103 mg, 1.02 mmol, 142 μL, 3.0 eq), EDCI (163 mg, 850 μmol, 2.5 eq) and HOBt (115 mg, 850 μmol, 2.5 eq). The mixture was stirred at 20° C. for 16 h. LCMS indicated that the starting material was completely consumed, and the desired mass was detected. The reaction mixture was poured into H₂O (10 mL) and extracted with DCM (10 mL×2). The combined organic layers were dried over Na₂SO₄, filtered, and concentrated under vacuum to give a residue which was purified by preparative HPLC (Phenomenex Luna C18 column (200×40 mm, 10 μm); flow rate: 25 mL/min; gradient: 10%-50% B over 8 min; mobile phase A: 0.2% aqueous FA, mobile phase B: acetonitrile). N-(1-(3-Methoxynaphthalen-1-yl)cyclopropyl)-2-methyl-5-((1-methylazetidin-2-yl)methoxy)benzamide (38.2 mg, 80.2 μmol, 24% yield, FA salt) was obtained as a white solid. M+H⁺=431.1 (LCMS); ¹H NMR (400 MHZ, DMSO-d₆) δ 9.06 (s, 1H), 8.54 (d, J=8.3 Hz, 1H), 8.20 (s, 1H), 7.83 (d, J=8.0 Hz, 1H), 7.49-7.35 (m, 3H), 7.23 (d, J=2.4 Hz, 1H), 7.03 (d, J=8.4 Hz, 1H), 6.83 (dd, J=2.8, 8.4 Hz, 1H), 6.61 (d, J=2.8 Hz, 1H), 3.91-3.86 (m, 5H), 3.36-3.25 (m, 2H), 2.84-2.76 (m, 1H), 2.25 (s, 3H), 2.02-1.81 (m, 5H), 1.33 (br s, 2H), 1.18-1.13 (m, 2H).

### Example 239: 5-(2-(Dimethylamino)ethoxy)-2-methyl-N-(1-(3-methylnaphthalen-1-yl)cyclopropyl)benzamide (Compound 401)

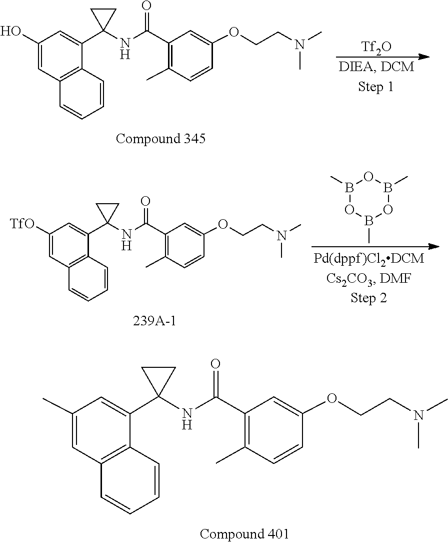

### Step 1:4-(1-(5-(2-(Dimethylamino)ethoxy)-2-methylbenzamido)cyclopropyl) naphthalen-2-yl trifluoromethanesulfonate (239A-1)

To a solution of 5-(2-(dimethylamino)ethoxy)-N-(1-(3-hydroxynaphthalen-1-yl)cyclopropyl)-2-methylbenzamide (110 mg, 272 μmol, 1.0 eq) in DCM (5.0 mL) were added DIEA (105 mg, 816 μmol, 142 μL, 3.0 eq) and Tf₂O (115 mg, 408 μmol, 67.3 μL, 1.5 eq) at −78° C. Then the mixture was stirred at −78° C. for 1 h. LCMS indicated that the starting material was completely consumed, and the desired mass was detected. The reaction mixture was allowed to warm to room temperature, poured into H₂O (10 mL) and extracted with DCM (5.0 mL×3). The combined organic layers were dried over Na₂SO₄, filtered, and concentrated under vacuum to give a residue which was purified by preparative TLC (DCM/MeOH=1/10, R_{f}=0.4). 4-(1-(5-(2-(Dimethylamino)ethoxy)-2-methylbenzamido)cyclopropyl) naphthalen-2-yl trifluoromethanesulfonate (130 mg, 242 μmol, 89% yield) was obtained as a brown solid. M+H⁺=537.2 (LCMS).

### Step 2: 5-(2-(dimethylamino)ethoxy)-2-methyl-N-(1-(3-methylnaphthalen-1-yl)cyclopropyl)benzamide (Compound 401)

To a solution of 4-(1-(5-(2-(dimethylamino)ethoxy)-2-methylbenzamido)cyclopropyl)naphthaen-2-yl trifluoromethanesulfonate (80.0 mg, 149 μmol, 1.0 eq) in DMF (4.0 mL) were added 2,4,6-trimethyl-1,3,5,2,4,6-trioxatriborinane (37.4 mg, 149 μmol, 41.7 μL, 50% purity, 1.0 eq), Cs₂CO₃ (160 mg, 492 μmol, 3.3 eq) and Pd(dppf)Cl₂·DCM (12.2 mg, 14.9 μmol, 0.1 eq). The mixture was stirred at 110° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (6.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 20%-55% B over 8 min; mobile phase A: 0.2% aqueous FA, mobile phase B: acetonitrile). 5-(2-(Dimethylamino)ethoxy)-2-methyl-N-(1-(3-methylnaphthalen-1-yl)cyclopropyl)benzamide (20.8 mg, 49.3 μmol, 33% yield, FA salt) was obtained as a gray solid. M+H⁺=403.1 (LCMS); ¹H NMR (400 MHZ, DMSO-d₆) § 9.05 (s, 1H), 8.63-8.56 (m, 1H), 7.85-7.79 (m, 1H), 7.64 (d, J=1.6 Hz, 1H), 7.59 (s, 1H), 7.52-7.43 (m, 2H), 7.03 (d, J=8.5 Hz, 1H), 6.83 (dd, J=2.8, 8.4 Hz, 1H), 6.59 (d, J=2.8 Hz, 1H), 3.94 (t, J=5.8 Hz, 2H), 2.55 (t, J=5.8 Hz, 2H), 2.47 (s, 3H), 2.17 (s, 6H), 1.98 (s, 3H), 1.37-1.30 (m, 2H), 1.19-1.13 (m, 2H).

### Example 240: 5-(2-Dimethylamino)ethoxy)-N-(1-(6-fluoronaphthalen-1-yl)cyclopropyl)-2-methylbenzamide (Compound 319)

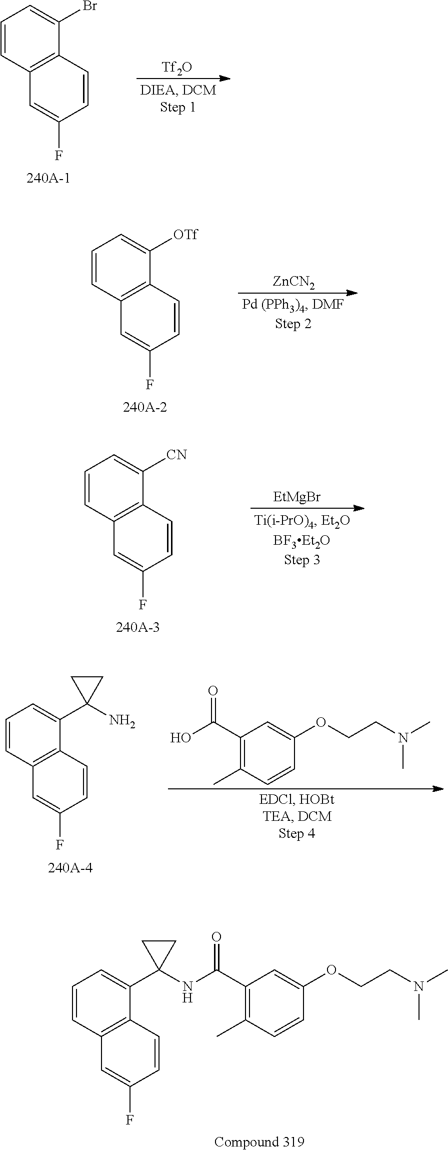

### Step 1: 6-Fluoronaphthalen-1-yl trifluoromethanesulfonate (240A-2)

To a solution of 6-fluoronaphthalen-1-ol (420 mg, 2.59 mmol, 1.0 eq) in DCM (12 mL) were added DIEA (669 mg, 5.18 mmol, 902 μL, 2.0 eq) and Tf₂O (730 mg, 2.59 mmol, 427 μL, 1.0 eq) at 0° C. The mixture was stirred at 0° C. for 1 h. TLC indicated that the starting material was completely consumed. The reaction mixture was allowed to warm to room temperature, poured into H₂O (20 mL) and extracted with DCM (20 mL×3). The combined organic layers were dried over Na₂SO₄, filtered, and concentrated under vacuum to give a residue which was purified by flash silica gel chromatography using a gradient of EtOAc/petroleum ether from 0/1 to 1/20. 6-Fluoronaphthalen-1-yl trifluoromethanesulfonate (750 mg, 2.55 mmol, 90% purity) was obtained as a white solid. ¹H NMR (400 MHZ, CDCl₃) δ 8.10 (br dd, J=5.3, 8.9 Hz, 1H), 7.83 (br d, J=8.2 Hz, 1H), 7.62-7.49 (m, 2H), 7.49-7.36 (m, 2H).

### Step 2: 6-Fluoro-1-naphthonitrile (240A-3)

To a solution of 6-fluoronaphthalen-1-yl trifluoromethanesulfonate (750 mg, 2.55 mmol, 1.0 eq) in DMF (8.0 mL) were added Zn(CN)₂ (598 mg, 5.10 mmol, 323 μL, 2.0 eq) and Pd(PPh₃)₄ (117 mg, 101 μmol, 0.04 eq) under a N₂ atmosphere. The mixture was stirred at 120° C. for 30 min. TLC indicated that the starting material was completely consumed. The reaction mixture was allowed to cool to room temperature, poured into saturated aqueous NH₄Cl (16 mL) and extracted with EtOAc (8.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/20. 6-Fluoro-1-naphthonitrile (390 mg, 2.28 mmol, 83% yield) was obtained as a white solid. ¹H NMR (400 MHz, CDCl₃) δ 8.26 (dd, J=5.3, 9.2 Hz, 1H), 8.04 (d, J=8.3 Hz, 1H), 7.90 (d, J=7.1 Hz, 1H), 7.62-7.54 (m, 2H), 7.49 (dt, J=2.5, 8.7 Hz, 1H).

### Step 3: 1-(6-Fluoronaphthalen-1-yl)cyclopropanamine (240A-4)

A mixture of 6-fluoro-1-naphthonitrile (200 mg, 1.17 mmol, 1.0 eq) in anhydrous Et₂O (35 mL) was degassed and purged with N₂ three times. To this mixture was added Ti(i-PrO)₄ (365 mg, 1.29 mmol, 379 μL, 1.1 eq) slowly at −78° C., and then EtMgBr (3 M in Et₂O, 856 μL, 2.2 eq) was added dropwise to maintain the temperature between −78° C. and −75° C. under a N₂ atmosphere. After the addition was complete, the resulting mixture was stirred at the same temperature for 10 min and warmed to 20° C. over 1 h. BF₃·Et₂O (331 mg, 2.34 mmol, 288 μL, 2.0 eq) was added slowly with no obvious temperature change. The mixture was stirred at 20° C. for another 1 h. LCMS indicated that the starting material was completely consumed, and the desired mass was detected. The reaction mixture was poured into a mixture of HCl (1 M aqueous) (20 mL) and MTBE (20 mL). The mixture was washed with MTBE (25 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (25 mL×4). The combined organic layers were dried over Na₂SO₄, filtered, and concentrated under vacuum to give a residue which was purified by preparative TLC (DCM/MeOH=1/10, R_{f}=0.5). 1-(6-Fluoro-1-naphthyl)cyclopropanamine (110 mg, 546 μmol, 46% yield) was obtained as a white solid. M+H⁺=202.1 (LCMS); ¹H NMR (400 MHZ, CDCl₃) δ 8.47 (dd, J=5.6, 9.3 Hz, 1H), 7.70 (d, J=8.1 Hz, 1H), 7.55-7.47 (m, 2H), 7.44 (d, J=7.7 Hz, 1H), 7.40-7.32 (m, 1H), 1.24-1.17 (m, 2H), 1.07-1.00 (m, 2H).

### Step 4: 5-(2-(Dimethylamino)ethoxy)-N-(1-(6-fluoronaphthalen-1-yl)cyclopropyl)-2-methylbenzamide (Compound 319)

To a solution of 1-(6-fluoro-1-naphthyl)cyclopropanamine (80.0 mg, 397 μmol, 1.0 eq) in DMF (4.0 mL) were added 5-(2-(dimethylamino)ethoxy)-2-methylbenzoic acid (133 mg, 596 μmol, 1.5 eq), TEA (120 mg, 1.19 mmol, 166 μL, 3.0 eq), EDCI (91.4 mg, 477 μmol, 1.2 eq) and HOBt (64.4 mg, 477 μmol, 1.2 eq). The mixture was stirred at 25° C. for 6 h. LCMS indicated that the starting material completely consumed, and the desired product was detected. The reaction mixture was poured into H₂O (4.0 mL) and extracted with EtOAc (2.0 mL×3). The combined EtOAc layers were dried over Na₂SO₄, filtered, and concentrated under vacuum to give a residue which was purified by preparative HPLC (Phenomenex Luna C18 column (75× 30 mm, 3 μm); flow rate: 25 mL/min; gradient: 20%-50% B over 8 min; mobile phase A: 0.2% aqueous FA, mobile phase B: acetonitrile). 5-(2-(Dimethylamino)ethoxy)-N-(1-(6-fluoronaphthalen-1-yl)cyclopropyl)-2-methylbenzamide (67.9 mg, 167 μmol, 42% yield, FA salt) was obtained as a white solid. M+H⁺=407.1 (LCMS); ¹H NMR (400 MHZ, CDCl₃) δ 8.59 (dd, J=5.6, 9.1 Hz, 1H), 8.43 (br s, 1H), 7.88 (d, J=7.0 Hz, 1H), 7.73 (d, J=8.3 Hz, 1H), 7.59-7.43 (m, 2H), 7.34 (dt, J=2.6, 8.8 Hz, 1H), 7.00 (d, J=8.3 Hz, 1H), 6.89-6.57 (m, 3H), 4.08 (t, J=5.3 Hz, 2H), 2.89 (t, J=5.3 Hz, 2H), 2.46 (s, 6H), 2.12 (s, 3H), 1.70-1.49 (m, 2H), 1.48-1.32 (m, 2H).

### Example 241: (S)-5-(Azetidin-2-ylmethoxy)-N-(1-(6-fluoronaphthalen-1-yl)cyclopropyl)-2-methylbenzamide (Compound 392)

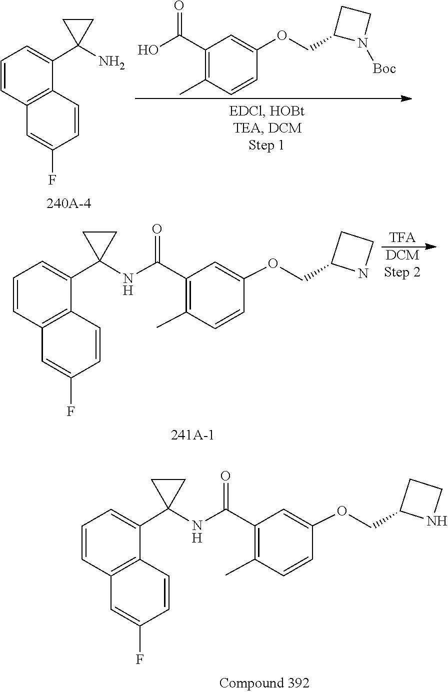

### Step 1: (S)-tert-butyl 2-((3-((1-(6-Fluoronaphthalen-1-yl)cyclopropyl)carbamoyl)-4-methylphenoxy)methyl)azetidine-1-carboxylate (241A-1)

To a solution of 1-(6-fluoronaphthalen-1-yl)cyclopropanamine (31.3 mg, 156 μmol, 1.0 eq) and (S)-5-((1-(tert-butoxycarbonyl)azetidin-2-yl)methoxy)-2-methylbenzoic acid (50.0 mg, 155 μmol, 1.0 eq) in DCM (2.0 mL) were added TEA (47.2 mg, 466 μmol, 64.9 μL, 3.0 eq), EDCI (44.7 mg, 233 μmol, 1.5 eq) and HOBt (31.5 mg, 233 μmol, 1.5 eq). The mixture was stirred at 20° C. for 16 h. LCMS indicated that the starting material was completely consumed, and the desired mass was detected. The reaction mixture was poured into H₂O (3.0 mL) and extracted with DCM (3.0 mL×3). The combined organic layers were dried over Na₂SO₄, filtered, and concentrated under vacuum to give a residue which was purified by preparative TLC (pure EtOAc, R_{f}=0.6). (S)-tert-Butyl 2-((3-((1-(6-fluoronaphthalen-1-yl)cyclopropyl) carbamoyl)-4-methylphenoxy)methyl)azetidine-1-carboxylate (70.0 mg, 139 μmol, 89% yield) was obtained as a white solid. M−56+H⁺=449.1 (LCMS).

### Step 2: (S)-5-(Azetidin-2-ylmethoxy)-N-(1-(6-fluoronaphthalen-1-yl)cyclopropyl)-2-methylbenzamide (Compound 392)

To a solution of(S)-tert-butyl 2-((3-((1-(6-fluoronaphthalen-1-yl)cyclopropyl)carbamoyl)-4-methylphenoxy)methyl)azetidine-1-carboxylate (70.0 mg, 139 μmol, 1.0 eq) in DCM (3.0 mL) was added TFA (2.16 g, 18.9 mmol, 1.40 mL, 136 eq). The mixture was stirred at 20° C. for 1 h. LCMS indicated that the starting material was completely consumed, and the desired mass was detected. The mixture was concentrated under vacuum at 20° C. to give a residue which was purified by preparative HPLC (C18-1 column (150×30 mm, 5 μm); flow rate: 25 mL/min; gradient: 15%-60% B over 8 min; mobile phase A: 0.1% aqueous TFA, mobile phase B: acetonitrile). (S)-5-(Azetidin-2-ylmethoxy)-N-(1-(6-fluoronaphthalen-1-yl)cyclopropyl)-2-methylbenzamide (10.7 mg, 20.6 μmol, 15% yield, TFA salt) as a white solid. M+H⁺=405.1 (LCMS); ¹H NMR (400 MHZ, DMSO-d₆) δ9.11 (s, 1H), 8.95-8.67 (m, 3H), 7.84 (d, J=8.3 Hz, 1H), 7.79 (d, J=7.0 Hz, 1H), 7.73 (dd, J=2.6, 10.3 Hz, 1H), 7.59-7.39 (m, 2H), 7.09 (d, J=8.5 Hz, 1H), 6.90 (dd, J=2.8, 8.4 Hz, 1H), 6.69 (d, J=2.8 Hz, 1H), 4.83-4.54 (m, 1H), 4.32-4.03 (m, 2H), 3.99-3.77 (m, 2H), 2.48-2.40 (m, 1H), 2.39-2.27 (m, 1H), 1.97 (s, 3H), 1.36 (s, 2H), 1.20 (br s, 2H).

### Example 242: N-(1-(6-Fluoronaphthalen-1-yl)cyclopropyl)-2-methyl-5-(2-(methylamino)ethoxy)benzamide (Compound 377)

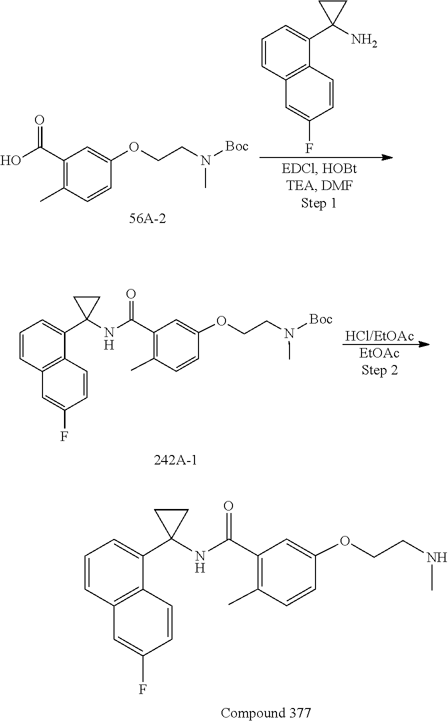 [SMILES: Cc1ccc(OCCN(C)C(=O)OC(C)(C)C)cc1C(=O)O ; NC1(c2cccc3cc(F)ccc23)CC1 ; Cc1ccc(OCCN(C)C(=O)OC(C)(C)C)cc1C(=O)NC1(c2cccc3cc(F)ccc23)CC1 ; CNCCOc1ccc(C)c(C(=O)NC2(c3cccc4cc(F)ccc34)CC2)c1]

### Step 1: tert-Butyl(2-(3-((1-(6-fluoronaphthalen-1-yl)cyclopropyl)carbamoyl)-4-methylphenoxy)ethyl)(methyl)carbamate (242A-1)

To a solution of 5-(2-((tert-butoxycarbonyl)(methyl)amino)ethoxy)-2-methylbenzoic acid (100 mg, 323 μmol, 1.0 eq) and 1-(6-fluoronaphthalen-1-yl)cyclopropanamine (65.1 mg, 323 μmol, 1.0 eq) in DMF (5.0 mL) were added TEA (98.1 mg, 970 μmol, 135 μL, 3.0 eq), EDCI (155 mg, 808 μmol, 2.5 eq) and HOBt (109 mg, 808 μmol, 2.5 eq). The mixture was stirred at 20° C. for 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by was purified by preparative TLC (EtOAc/petroleum ether=1/2, R_{f}=0.4). tert-Butyl(2-(3-((1-(6-fluoronaphthalen-1-yl)cyclopropyl)carbamoyl)-4-methylphenoxy)ethyl)(methyl)carbamate (100 mg, 203 μmol, 63% yield) was obtained as a colorless oil. M+H⁺=493.2 (LCMS); ¹H NMR (400 MHZ, DMSO-d₆) δ 9.10 (s, 1H), 8.73 (dd, J=5.4, 9.2 Hz, 1H), 7.83 (d, J=8.1 Hz, 1H), 7.77 (d, J=7.0 Hz, 1H), 7.72 (dd, J=2.4, 10.3 Hz, 1H), 7.50 (t, J=7.8 Hz, 1H), 7.47-7.42 (m, 1H), 7.04 (br d, J=8.0 Hz, 1H), 6.84 (dd, J=2.5, 8.5 Hz, 1H), 6.60 (br s, 1H), 4.00-3.94 (m, 2H), 3.46 (t, J=5.6 Hz, 2H), 2.82 (br d, J=11.0 Hz, 3H), 1.95 (s, 3H), 1.42-1.23 (m, 13H).

### Step 2: N-(1-(6-Fluoronaphthalen-1-yl)cyclopropyl)-2-methyl-5-(2-(methylamino)ethoxy)benzamide (Compound 377)

To a stirred solution of tert-butyl(2-(3-((1-(6-fluoronaphthalen-1-yl)cyclopropyl)carbamoyl)-4-methyl phenoxy)ethyl)(methyl)carbamate (100 mg, 203 μmol, 1.0 eq) in EtOAc (2.0 mL) was added HCl/EtOAc (4 M, 10.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna (80×30 mm, 3 μm); flow rate: 25 mL/min; gradient: 5%-40% B over 8 min; mobile phase A: 0.04% aqueous HCl, mobile phase B: acetonitrile). N-(1-(6-Fluoronaphthalen-1-yl)cyclopropyl)-2-methyl-5-(2-(methylamino) ethoxy)benzamide (40.7 mg, 94.9 μmol, 47% yield, HCl salt) was obtained as a white solid. M+H⁺=393.1 (LCMS); ¹H NMR (400 MHZ, DMSO-d₆) δ 9.13 (s, 1H), 9.01 (br s, 2H), 8.68 (br dd, J=5.9, 9.1 Hz, 1H), 7.75 (br dd, J=7.6, 18.0 Hz, 2H), 7.67 (dd, J=2.1, 10.2 Hz, 1H), 7.49-7.38 (m, 2H), 7.01 (d, J=8.4 Hz, 1H), 6.83 (dd, J=2.2, 8.3 Hz, 1H), 6.62 (d, J=2.0 Hz, 1H), 4.11 (br t, J=4.3 Hz, 2H), 3.18 (br s, 2H), 2.44 (br s, 3H), 1.89 (s, 3H), 1.30 (br s, 2H), 1.12 (br s, 2H).

### Example 243: (S)—N-(1-(6-Fluoronaphthalen-1-yl)cyclopropyl)-2-methyl-5-((1-methylazetidin-2-yl)methoxy)benzamide (Compound 389)

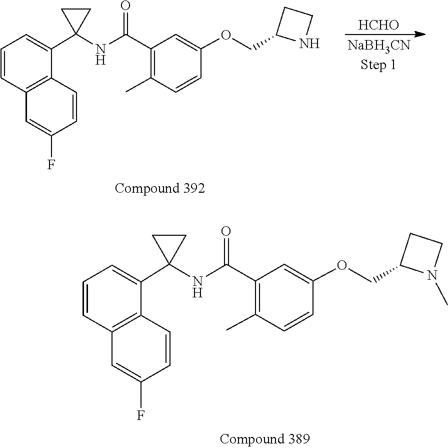 [SMILES: Cc1ccc(OC[C@@H]2CCN2)cc1C(=O)NC1(c2cccc3cc(F)ccc23)CC1 ; Cc1ccc(OC[C@@H]2CCN2C)cc1C(=O)NC1(c2cccc3cc(F)ccc23)CC1]

### Step 1: (S)—N-(1-(6-Fluoronaphthalen-1-yl)cyclopropyl)-2-methyl-5-((1-methylazetidin-2-yl)methoxy)benzamide (Compound 389)

To a solution of(S)-5-(azetidin-2-ylmethoxy)-N-(1-(6-fluoronaphthalen-1-yl)cyclopropyl)-2-methylbenzamide (150 mg, 289 μmol, 1.0 eq, TFA salt) in MeOH (2.0 mL) was added TEA (40.0 μL), followed by the addition of formaldehyde (47.0 mg, 579 mmol, 43.1 μL, 37% purity in water, 2.0 eq). The resulting mixture was adjusted to pH 6 with a small amount of AcOH. The mixture was stirred at 20° C. for 30 min, then NaBH₃CN (36.4 mg, 579 μmol, 2.0 eq) was added. The reaction mixture was stirred at 20° C. for another 3 h. LCMS indicated that the starting material was completely consumed, and the desired mass was detected. The reaction mixture was poured into H₂O (3.0 mL) and extracted with EtOAc (3.0 mL×3). The combined organic layers were dried over Na₂SO₄, filtered, and concentrated under vacuum to give a residue which was purified by preparative HPLC (Phenomenex Luma C18 column (75×30 mm, 3 μm); flow rate: 25 mL/min; gradient: 20%-55% B over 8 min; mobile phase A: 0.2% aqueous FA, mobile phase B: acetonitrile). (S)—N-(1-(6-Fluoronaphthalen-1-yl)cyclopropyl)-2-methyl-5-((1-methylazetidin-2-yl)methoxy)benzamide (10.7 mg, 13.0 μmol, 8% yield, FA salt) was obtained as a white solid. M+H⁺=419.1 (LCMS); ¹H NMR (400 MHZ, DMSO-d₆) δ9.08 (s, 1H), 8.74 (dd, J=5.8, 9.3 Hz, 1H), 8.22 (s, 1H), 7.87-7.66 (m, 3H), 7.56-7.40 (m, 2H), 7.02 (d, J=8.4 Hz, 1H), 6.83 (dd, J=2.8, 8.4 Hz, 1H), 6.60 (d, J=2.6 Hz, 1H), 3.86 (d, J=5.5 Hz, 2H), 3.27-3.18 (m, 3H), 2.71 (td, J=8.1, 16.0 Hz, 1H), 2.21 (s, 3H), 1.95 (s, 3H), 1.90-1.77 (m, 1H), 1.36 (s, 2H), 1.22-1.09 (m, 2H).

### Example 244: 5-(2-(Dimethylamino)ethoxy)-N-(1-(7-fluoronaphthalen-1-yl)cyclopropyl)-2-methylbenzamide (Compound 365)

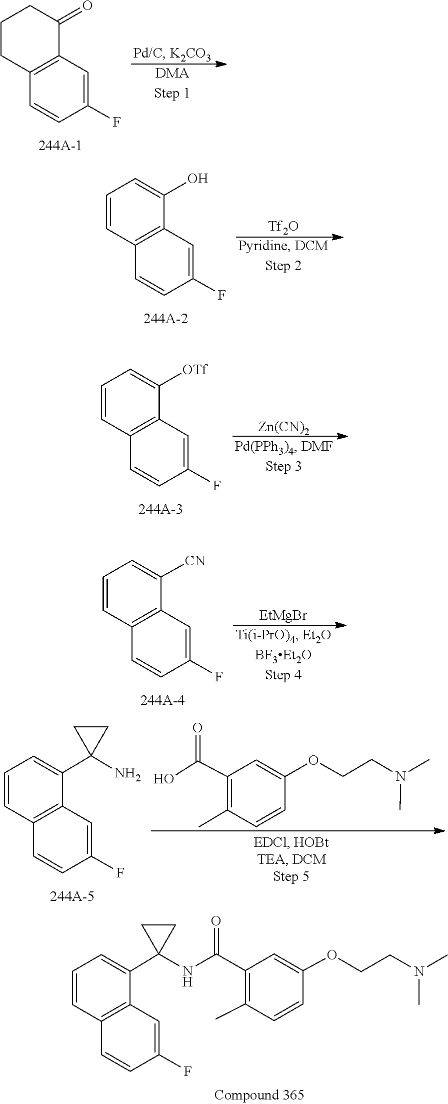

### Step 1: 7-Fluoronaphthalen-1-ol (244A-2)

To a solution of 7-fluoro-3,4-dihydronaphthalen-1 (2H)-one (1.00 g, 6.09 mmol, 1.0 eq) in DMA (10 mL) was added 10% palladium on carbon (100 mg, 1.83 mmol, 0.3 eq) and K₂CO₃ (2.53 g, 18.3 mmol, 3.0 eq). The mixture was stirred at 160° C. for 2 h. TLC indicated that the starting material was completely consumed. The reaction mixture was allowed to cool to room temperature and filtered through a pad of Celite. The filtrate was poured into H₂O (50 mL) and extracted with EtOAc (25 mL×4). The combined organic layers were dried over Na₂SO₄, filtered, and concentrated under vacuum to give a residue which was purified by flash silica gel chromatography using a gradient of EtOAc/petroleum ether from 0/1 to 1/5. 7-Fluoronaphthalen-1-ol (900 mg, 5.55 mmol, 91% yield) was obtained as a white solid. ¹H NMR (400 MHZ, CDCl₃) δ 8.23-8.15 (m, 1H), 7.87-7.78 (m, 1H), 7.54-7.49 (m, 1H), 7.48-7.43 (m, 1H), 7.35-7.29 (m, 1H), 6.87-6.80 (m, 1H), 5.24-5.18 (m, 1H).

### Step 2: 7-Fluoronaphthalen-1-yl trifluoromethanesulfonate (244A-3)

To a solution of 7-fluoronaphthalen-1-ol (350 mg, 2.16 mmol, 1.0 eq) in DCM (20 mL) were added Tf₂O (670 mg, 2.37 mmol, 392 μL, 1.0 eq) and pyridine (588 mg, 7.43 mmol, 0.6 mL, 3.4 eq) at 0° C. The mixture was stirred at 0° C. for 2 h. TLC indicated that the starting material was completely consumed. The reaction mixture was poured into H₂O (20 mL) and extracted with DCM (20 mL×3). The combined layers were dried over Na₂SO₄, filtered, and concentrated under vacuum to give a residue which was purified by flash silica gel chromatography using a gradient of EtOAc/petroleum ether from 0/1 to 1/5. 7-Fluoronaphthalen-1-yl trifluoromethane sulfonate (600 mg, 2.04 mmol, 94% yield) was obtained as a yellow oil. ¹H NMR (400 MHZ, CDCl₃) δ 7.97-7.87 (m, 2H), 7.72-7.66 (m, 1H), 7.55-7.45 (m, 2H), 7.43-7.36 (m, 1H).

### Step 3: 7-Fluoro-1-naphthonitrile (244A-4)

To a solution of 7-fluoronaphthalen-1-yl trifluoromethanesulfonate (500 mg, 1.70 mmol, 1.0 eq) in DMF (8.0 mL) were added Zn(CN)₂ (399 mg, 3.40 mmol, 216 μL, 2.0 eq) and Pd(PPh₃)₄ (196 mg, 170 μmol, 0.1 eq). The mixture was stirred at 110° C. for 18 h under a N₂ atmosphere. TLC indicated that the starting material was completely consumed. The reaction mixture was allowed to cool to room temperature, poured into saturated aqueous NH₄Cl (2.0 mL) and extracted with EtOAc (2.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/5. 7-Fluoro-1-naphthonitrile (150 mg, 876 μmol, 52% yield) was obtained as a white solid. ¹H NMR (400 MHz, CDCl₃) δ8.13-8.07 (m, 1H), 7.99-7.92 (m, 2H), 7.91-7.84 (m, 1H), 7.56-7.49 (m, 1H), 7.46-7.38 (m, 1H).

### Step 4: 1-(7-Fluoronaphthalen-1-yl)cyclopropanamine (244A-5)

A mixture of 7-fluoro-1-naphthonitrile (45.0 mg, 263 μmol, 1.0 eq) in anhydrous Et₂O (10 mL) was degassed and purged with N₂ three times. The mixture was stirred at −78° C. To this mixture was added Ti(i-PrO)₄ (82.2 mg, 289 μmol, 85.3 μL, 1.1 eq) slowly, and then EtMgBr (3 M in Et₂O, 193 μL, 2.2 eq) was added dropwise to maintain the temperature between −78° C. and −75° C. over 5 min under a N₂ atmosphere. After the addition was complete, the resulting mixture was stirred at the same temperature for 10 min and warmed to 20° C. over 1 h. BF₃·Et₂O (74.6 mg, 526 μmol, 64.9 μL, 2.0 eq) was added slowly with no obvious temperature change. The mixture was stirred at 20° C. for another 1 h. LCMS indicated that the starting material was completely consumed, and the desired mass was detected. The reaction mixture was poured into a mixture of HCl (1 M aqueous) (5.0 mL) and MTBE (5.0 mL), and extracted with MTBE (5.0 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (5.0 mL×4). The combined organic layers were dried over Na₂SO₄, filtered, and concentrated under vacuum to give a residue which was purified by preparative TLC (pure EtOAc, R_{f}=0.3). 1-(7-Fluoronaphthalen-1-yl)cyclopropane mine (30.0 mg, 149 μmol, 57% yield) was obtained as a yellow oil. M+H⁺=202.1 (LCMS).

### Step 5: 5-(2-(Dimethylamino)ethoxy)-N-(1-(7-fluoronaphthalen-1-yl)cyclopropyl)-2-methylbenzamide (Compound 365)

To a solution of 1-(7-fluoronaphthalen-1-yl)cyclopropanamine (20.0 mg, 49.7 μmol, 1.0 eq) in DCM (1.0 mL) was added 5-(2-(dimethylamino)ethoxy)-2-methylbenzoic acid (111 mg, 49.7 μmol, 1.0 eq), TEA (15.1 mg, 149 μmol, 20.8 μL, 3.0 eq), EDCI (23.8 mg, 124 μmol, 2.5 eq) and HOBt (16.8 mg, 124 μmol, 2.5 eq). The mixture was stirred at 20° C. for 16 h. LCMS indicated that the starting material completely consumed, and the desired mass was detected. The reaction mixture was poured into H₂O (5.0 mL) and extracted with DCM (5.0 mL×5). The combined layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 30%-60% B over 8 min; mobile phase A: 0.2% aqueous FA, mobile phase B: acetonitrile). 5-(2-(Dimethylamino)ethoxy)-N-(1-(7-fluoronaphthalen-1-yl)cyclopropyl)-2-methylbenzamide (8.20 mg, 18.0 μmol, 36% yield, FA salt) was obtained as a white solid. M+H⁺=407.1 (LCMS); ¹H NMR (400 MHZ, CDCl₃) δ 8.43-8.40 (m, 1H), 8.12-8.07 (m, 1H), 8.02-7.96 (m, 1H), 7.92-7.85 (m, 1H), 7.82-7.76 (m, 1H), 7.47-7.42 (m, 1H), 7.32-7.28 (m, 1H), 7.04-6.99 (m, 1H), 6.83-6.74 (m, 2H), 6.59-6.56 (m, 1H), 4.08-4.00 (m, 2H), 2.84-2.76 (m, 2H), 2.42-2.38 (m, 6H), 2.19-2.10 (m, 3H), 1.61-1.52 (m, 2H), 1.43-1.33 (m, 2H).

### Example 245: 5-(Azetidin-3-ylamino)-2-methyl-N-(3-(5-(pyrrolidin-1-ylmethyl)thiophen-2-yl)benzyl)benzamide (Compound 120)

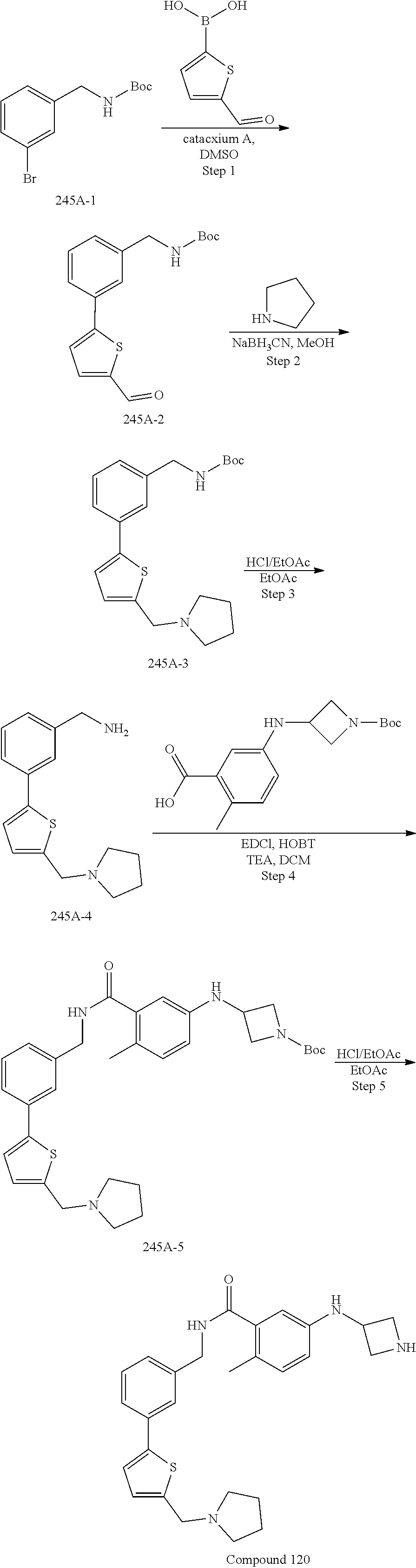

### Step 1: tert-Butyl 3-(5-formylthiophen-2-yl)benzylcarbamate (245A-2)

A mixture of tert-butyl 3-bromobenzylcarbamate (300 mg, 1.05 mmol, 1.0 eq), (5-formylthiophen-2-yl) boronic acid (196 mg, 1.26 mmol, 1.2 eq) and KOAc (308 mg, 3.15 mmol, 3.0 eq) in DMSO (6.0 mL) was degassed and purged with N₂ three times. To the mixture were added cataCXium® A (70.6 mg, 210 μmol, 0.2 eq) and Pd(OAc) 2 (23.5 mg, 105 μmol, 0.1 eq). The resulting mixture was stirred at 80° C. for 6 h under a N₂ atmosphere. LCMS indicated that the starting material was completely consumed, and the desired mass was detected. The reaction mixture was allowed to cool to room temperature, poured into H₂O (5.0 mL) and extracted with EtOAc (5.0 mL×3). The combined organic layers were dried over Na₂SO₄, filtered, and concentrated under vacuum to give a residue which was purified by preparative TLC (EtOAc/petroleum ether=1/1, R_{f}=0.5). tert-Butyl 3-(5-formylthiophen-2-yl)benzylcarbamate (150 mg, 473 μmol, 45% yield) was obtained as a brown oil. M+H⁺=318.3 (LCMS).

### Step 2: tert-Butyl 3-(5-(pyrrolidin-1-ylmethyl)thiophen-2-yl)benzylcarbamate (245A-3)

To a solution of tert-butyl 3-(5-formylthiophen-2-yl)benzylcarbamate (150 mg, 473 μmol, 1.0 eq) in MeOH (5.0 mL) was added TEA (10.0 μL), followed by the addition of pyrrolidine (67.2 mg, 945 μmol, 78.9 μL, 2.0 eq). The resulting mixture was adjusted to pH 6 with a small amount of AcOH. The mixture was stirred at 20° C. for 30 min, then NaBH₃CN (44.5 mg, 708 μmol, 1.5 eq) was added. The resulting reaction mixture was stirred at 20° C. for another 2 h. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/5. tert-Butyl 3-(5-(pyrrolidin-1-ylmethyl)thiophen-2-yl)benzylcarbamate (90.0 mg, 242 μmol, 51% yield) was obtained as a yellow oil. M+H⁺=373.1 (LCMS).

### Step 3: (3-(5-(Pyrrolidin-1-ylmethyl)thiophen-2-yl)phenyl) methanamine (245A-4)

To a stirred solution of tert-butyl 3-(5-(pyrrolidin-1-ylmethyl)thiophen-2-yl)benzylcarbamate (90.0 mg, 242 μmol, 1.0 eq) in EtOAc (5.0 mL) was added HCl/EtOAc (4 M, 3.0 mL). The mixture was stirred at 20° C. for 2 h. LCMS indicated that the starting material was completely consumed, and the desired mass was detected. The mixture was concentrated under vacuum at 30° C. to give (3-(5-(Pyrrolidin-1-ylmethyl)thiophen-2-yl)phenyl) methanamine (40.0 mg, 147 μmol, 61% yield, HCl salt) as a yellow oil. M+H⁺=273.1 (LCMS).

### Step 4: tert-Butyl 3-((4-methyl-3-((3-(5-(pyrrolidin-1-ylmethyl)thiophen-2-yl)benzyl)carbamoyl)phenyl)amino)azetidine-1-carboxylate (245A-5)

To a solution of (3-(5-(pyrrolidin-1-ylmethyl)thiophen-2-yl)phenyl) methanamine (40.0 mg, 147 μmol, 1.0 eq) and 5-((1-(tert-butoxycarbonyl)azetidin-3-yl)amino)-2-methylbenzoic acid (45.0 mg, 147 μmol, 1.0 eq) in DCM (5.0 mL) were added TEA (44.6 mg, 441 μmol, 61.3 μL, 3 eq), EDCI (33.7 mg, 176 μmol, 1.2 eq) and HOBt (23.8 mg, 176 μmol, 1.2 eq). The mixture was stirred at 20° C. for 16 h. LCMS indicated that the starting material was completely consumed, and the desired mass was detected. The reaction mixture was poured into H₂O (5.0 mL) and extracted with DCM (5.0 mL×2). The combined organic layers were dried over Na₂SO₄, filtered, and concentrated under vacuum to give a residue which was purified by flash silica gel chromatography using a gradient of MeOH/DCM from 0/1 to 1/50. tert-Butyl 3-((4-methyl-3-((3-(5-(pyrrolidin-1-ylmethyl)thiophen-2-yl)benzyl)carbamoyl)phenyl)amino) azetidine-1-carboxylate (40.0 mg, 71.3 μmol, 59% yield) was obtained as a yellow solid. M+H⁺=561.2 (LCMS).

### Step 5: 5-(Azetidin-3-ylamino)-2-methyl-N-(3-(5-(pyrrolidin-1-ylmethyl)thiophen-2-yl)benzyl)benzamide (Compound 120)

To a stirred solution of tert-butyl 3-((4-methyl-3-((3-(5-(pyrrolidin-1-ylmethyl)thiophen-2yl)benzyl)carbamoyl)phenyl)amino) azetidine-1-carboxylate (40.0 mg, 71.3 μmol, 1.0 eq) in EtOAc (2.0 mL) was added HCl/EtOAc (4 M, 3.0 mL). The mixture was stirred at 20° C. for 2 h. LCMS indicated that the starting material was completely consumed, and the desired mass was detected. The mixture was concentrated under vacuum at 30° C. to give a residue which was purified by preparative HPLC (Phenomenex Gemini C18 column (100×40 mm, 5 μm); flow rate: 25 mL/min; gradient: 10%-55% B over 8 min; mobile phase A: 0.1% aqueous TFA, mobile phase B: acetonitrile). 5-(Azetidin-3-ylamino)-2-methyl-N-(3-(5-(pyrrolidin-1-ylmethyl)thiophen-2-yl)benzyl)benzamide (9.20 mg, 19.9 μmol, 28% yield, TFA salt) was obtained as a white solid. M+H⁺=461.0 (LCMS); ¹H NMR (400 MHz, CD₃OD) δ 7.69 (s, 1H), 7.58-7.47 (m, 1H), 7.41-7.37 (m, 3H), 7.31-7.30 (m, 1H), 7.03-7.01 (m, 1H), 6.60-6.58 (m, 2H), 4.90-4.62 (m, 4H), 4.57-4.52 (m, 1H), 4.50-4.34 (m, 2H), 3.97-3.92 (m, 2H), 3.58 (s, 2H), 3.31-3.22 (m, 2H), 2.27 (s, 3H), 2.23-1.85 (m, 4H).

### Example 246: (R)-5-Methyl-N-(1-(3-(5-(pyrrolidin-1-ylmethyl)thiophen-2-yl)phenyl)ethyl)-1H-benzo[d]imidazole-6-carboxamide (Compound 126)

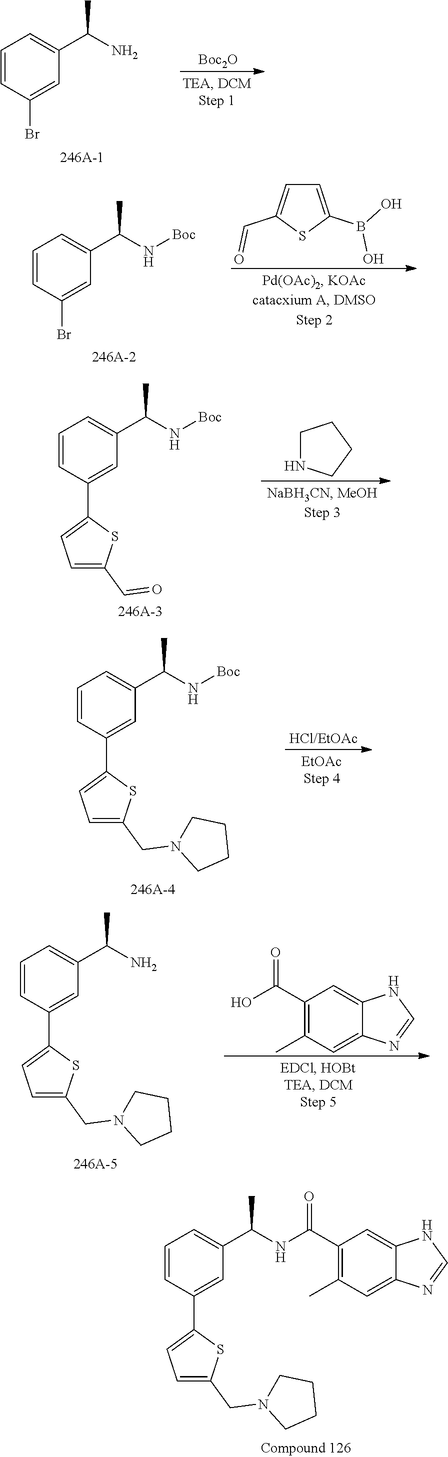

### Step 1: (R)-tert-Butyl(1-(3-bromophenyl)ethyl)carbamate (246A-2)

To a solution of (R)-1-(3-bromophenyl)ethanamine (1.00 g, 5.00 mmol, 1.0 eq) and TEA (759 mg, 7.50 mmol, 1.02 mL, 1.5 eq) in DCM (25 mL) was added Boc₂O (1.09 g, 5.00 mmol, 1.23 mL, 1.0 eq). The mixture was stirred at 20° C. for 4 h. TLC indicated that the starting material was completely consumed. The mixture was concentrated under vacuum to give a residue which was purified by flash silica gel chromatography using a gradient of EtOAc/petroleum ether from 1/100 to 1/10. (R)-tert-Butyl(1-(3-bromophenyl)ethyl)carbamate (1.40 g, 4.66 mmol, 93% yield) was obtained as a white solid. ¹H NMR (400 MHZ, CDCl₃) δ 7.44 (s, 1H), 7.38 (td, J=1.8, 7.3 Hz, 1H), 7.26-7.16 (m, 2H), 4.78 (br s, 2H), 1.51-1.33 (m, 12H).

### Step 2: (R)-tert-Butyl(1-(3-(5-formylthiophen-2-yl)phenyl)ethyl)carbamate (246A-3)

To a mixture of (R)-tert-butyl(1-(3-bromophenyl)ethyl)carbamate (100 mg, 333 μmol, 1.0 eq), (5-formyl-2-thienyl) boronic acid (52.0 mg, 333 μmol, 1.0 eq), KOAc (98.1 mg, 999 μmol, 3.0 eq), Pd(OAc) 2 (7.48 mg, 33.3 μmol, 0.1 eq) and cataCXium® A (23.9 mg, 66.6 μmol, 0.2 eq) in DMSO (5.0 mL) was degassed and purged with N₂ for three times. The mixture was stirred at 80° C. for 6 h under a N₂ atmosphere. TLC indicated that the starting material was completely consumed. The reaction mixture was allowed to cool to room temperature, poured into H₂O (20 mL) and extracted with EtOAc (10 mL×2). The combined organic layers were dried over Na₂SO₄, filtered, and concentrated under vacuum to give a residue which was purified by preparative TLC (EtOAc/petroleum ether=1/3, R_{f}=0.3). (R)-tert-Butyl(1-(3-(5-formylthiophen-2-yl)phenyl)ethyl)carbamate (50.0 mg, 151 μmol, 45% yield) was obtained as a yellow solid.

### Step 3: (R)-tert-Butyl(1-(3-(5-(pyrrolidin-1-ylmethyl)thiophen-2-yl)phenyl)ethyl)carbamate (246A-4)

To a mixture of (R)-tert-butyl(1-(3-(5-formylthiophen-2-yl)phenyl)ethyl)carbamate (50.0 mg, 151 μmol, 1.0 eq) and pyrrolidine (21.5 mg, 302 μmol, 25.2 μL, 2.0 eq) in MeOH (4.0 mL) was added NaBH₃CN (28.4 mg, 453 μmol, 3.0 eq). The mixture was stirred at 20° C. for 16 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give the crude product (R)-tert-butyl(1-(3-(5-(pyrrolidin-1-ylmethyl)thiophen-2-yl)phenyl)ethyl)carbamate (55.0 mg, 80% purity) as a brown oil, which was used in the next step without any further purification. M+H⁺=387.2 (LCMS).

### Step 4: (R)-1-(3-(5-(Pyrrolidin-1-ylmethyl)thiophen-2-yl)phenyl)ethanamine (246A-5)

To a mixture of (R)-tert-butyl(1-(3-(5-(pyrrolidin-1-ylmethyl)thiophen-2-yl)phenyl)ethyl) carbamate (55.0 mg, 142 μmol, 1.0 eq) in EtOAc (1.0 mL) was added HCl/EtOAc (4 M, 3.0 mL). The mixture was stirred at 20° C. for 2 h. LCMS indicated that the starting material was completely consumed, and the desired mass was detected. The mixture was concentrated under vacuum at 30° C. to give the crude product (R)-1-(3-(5-(pyrrolidin-1-ylmethyl)thiophen-2-yl)phenyl)ethanamine (50.0 mg, 80% purity) as a brown solid, which was used in the next step without any further purification. M+H⁺=287.3 (LCMS).

### Step 5: (R)-5-Methyl-N-(1-(3-(5-(pyrrolidin-1-ylmethyl)thiophen-2-yl)phenyl)ethyl)-1H-benzo[d]imidazole-6-carboxamide (Compound 126)

To a solution of (R)-1-(3-(5-(pyrrolidin-1-ylmethyl)thiophen-2-yl)phenyl)ethanamine (50.0 mg, 175 μmol, 1.0 eq) and 5-methyl-1H-benzo[d]imidazole-6-carboxylic acid (40.8 mg, 192 μmol, 1.1 eq) in DCM (2.0 mL) were added TEA (88.3 mg, 873 μmol, 122 μL, 5.0 eq), EDCI (40.2 mg, 209 μmol, 1.2 eq) and HOBt (28.3 mg, 209 μmol, 1.2 eq). The mixture was stirred at 20° C. for 3 h. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 25%-55% B over 8 min; mobile phase A: 10 mM aqueous NH₄HCO₃, mobile phase B: acetonitrile). (R)-5-Methyl-N-(1-(3-(5-(pyrrolidin-1-ylmethyl)thiophen-2-yl)phenyl)ethyl)-1H-benzo[d]imidazole-6-carboxamide (9.10 mg, 20.0 μmol, 12% yield) was obtained as a white solid. M+H⁺=445.2 (LCMS); ¹H NMR (400 MHZ, CD₃OD) δ 8.18 (s, 1H), 7.70 (s, 1H), 7.56-7.49 (m, 1H), 7.41-7.34 (m, 3H), 7.28 (d, J=3.6 Hz, 1H), 7.01 (d, J=3.6 Hz, 1H), 5.26 (q, J=7.0 Hz, 1H), 3.94 (s, 2H), 2.73 (br s, 4H), 2.47 (s, 3H), 1.86 (br s, 4H), 1.58 (d, J=7.0 Hz, 3H).

### Example 247: N—((R)-1-(3-(5-((((1S,3R)-3-hydroxycyclopentyl)amino)methyl)thiophen-2-yl)phenyl)ethyl)-5-methyl-1H-benzo[d]imidazole-6-carboxamide (Compound 134)

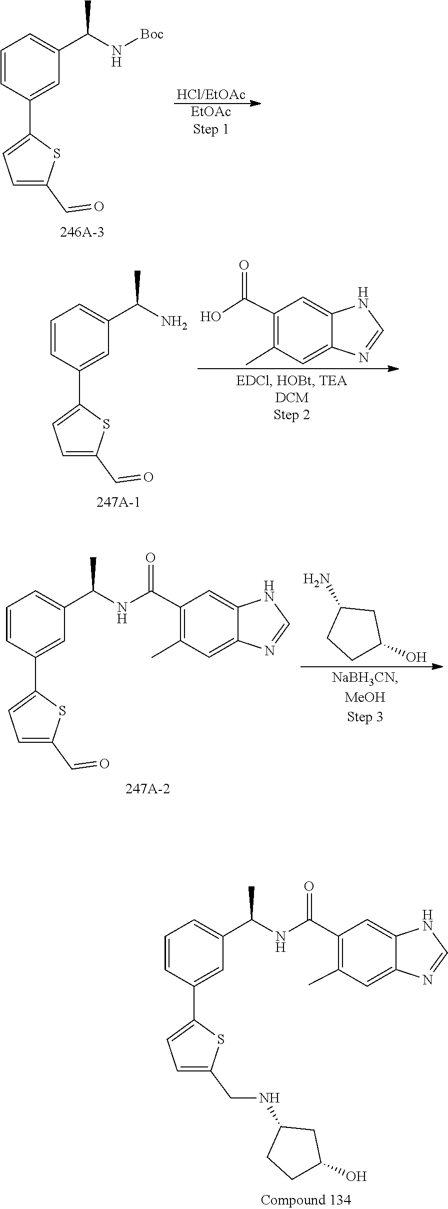

### Step 1: (R)-5-(3-(1-Aminoethyl)phenyl)thiophene-2-carbaldehyde (247A-1)

To a stirred solution of (R)-tert-butyl(1-(3-(5-formylthiophen-2-yl)phenyl)ethyl)carbamate (40.0 mg, 121 μmol, 1.0 eq) in EtOAc (3.0 mL) was added HCl/EtOAc (4 M, 3.0 mL). The mixture was stirred at 20° C. for 2 h. LCMS indicated that the starting material was completely consumed, and the desired mass was detected. The mixture was concentrated under vacuum at 30° C. to give (R)-5-(3-(1-aminoethyl)phenyl)thiophene-2-carbaldehyde (30 mg, 110 μmol, 91% yield, HCl salt) as a white solid. M+H⁺=232.1 (LCMS).

### Step 2: (R)—N-(1-(3-(5-Formylthiophen-2-yl)phenyl)ethyl)-5-methyl-1H-benzo[d]imidazole-6-carboxamide (247A-2)

To a solution of (R)-5-(3-(1-aminoethyl)phenyl)thiophene-2-carbaldehyde (100 mg, 432 μmol, 1.0 eq) and 5-methyl-1H-benzo[d]imidazole-6-carboxylic acid (101 mg, 476 μmol, 1.1 eq) in DCM (5.0 mL) were added TEA (131 mg, 1.30 mmol, 181 μL, 3 eq), EDCI (99.0 mg, 519 μmol, 1.2 eq) and HOBt (70.1 mg, 519 μmol, 1.2 eq). The mixture was stirred at 20° C. for 3 h. LCMS indicated that the starting material was completely consumed, and the desired mass was detected. The reaction mixture was poured into H₂O (5.0 mL) and extracted with DCM (5.0 mL×2). The combined organic layers were dried over Na₂SO₄, filtered, and concentrated under vacuum to give a residue which was purified by preparative TLC (EtOAc/petroleum ether=1/2, R_{f}=0.5). (R)—N-(1-(3-(5-Formylthiophen-2-yl)phenyl)ethyl)-5-methyl-1H-benzo[d]imidazole-6-carboxamide (100 mg, 257 μmol, 60% yield) was obtained as a yellow solid. M+H⁺=390.1 (LCMS).

### Step 3: N—((R)-1-(3-(5-((((1S,3R)-3-Hydroxycyclopentyl)amino)methyl)thiophen-2-yl)phenyl)ethyl)-5-methyl-1H-benzo[d]imidazole-6-carboxamide (Compound 134)

To a solution of (1R,3S)-3-aminocyclopentanol (27.2 mg, 198 μmol, 25.2 μL, 1.1 eq) in MeOH (3.0 mL) was added TEA (10.0 μL), followed by the addition of (R)—N-(1-(3-(5-formylthiophen-2-yl)phenyl)ethyl)-5-methyl-1H-benzo[d]imidazole-6-carboxamide (70.0 mg, 180 μmol, 1.0 eq). The resulting mixture was adjusted to pH 6 with a small amount of AcOH. The mixture was stirred at 20° C. for 30 min, then NaBH₃CN (22.6 mg, 359 μmol, 2.0 eq) was added. The reaction mixture was stirred at 20° C. for another 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Gemini C18 column (100×40 mm, 5 μm); flow rate: 25 mL/min; gradient: 1%-40% B over 8 min; mobile phase A: 0.2% aqueous FA, mobile phase B: acetonitrile). N—((R)-1-(3-(5-((((1S,3R)-3-Hydroxycyclopentyl)amino)methyl)thiophen-2-yl)phenyl)ethyl)-5-methyl-1H-benzo[d]imidazole-6-carboxamide (3.40 mg, 7.16 μmol, 4% yield) was obtained as a colorless gum. M+H⁺=475.2 (LCMS); ¹HNMR (400 MHZ, CD₃OD) δ 8.57-8.45 (m, 1H), 8.24-8.16 (m, 1H), 7.76-7.64 (m, 2H), 7.59-7.53 (m, 1H), 7.49-7.44 (m, 1H), 7.41 (dd, J=4.3, 11.4 Hz, 3H), 7.32-7.22 (m, 1H), 5.29-5.24 (m, 1H), 4.40 (s, 2H), 4.32 (td, J=4.1, 8.6 Hz, 1H), 3.68-3.58 (m, 1H), 2.54-2.42 (m, 3H), 2.30-2.09 (m, 2H), 2.01-1.76 (m, 4H), 1.66 (br d, J=6.9 Hz, 3H).

### Example 248: N—((R)-1-(3-(5-((((1S,3R)-3-Hydroxycyclopentyl)amino)methyl)thiophen-2-yl)phenyl)ethyl)-2-methyl-5-((1-methyl-1H-pyrazol-4-yl)amino)benzamide (Compound 137)

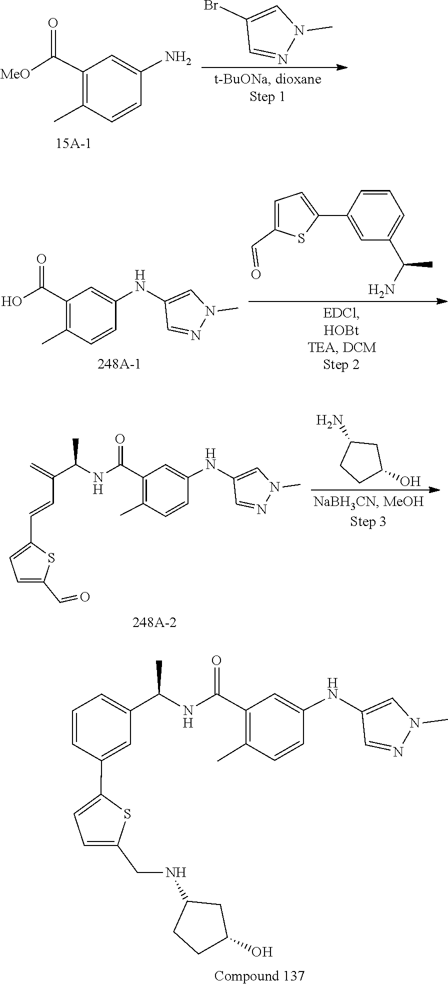

### Step 1: 2-Methyl-5-((1-methyl-1H-pyrazol-4-yl)amino)benzoic acid (248A-1)

To a stirred solution of 5-amino-2-methylbenzoate (200 mg, 1.21 mmol, 1.0 eq) and 4-bromo-1-methyl-1H-pyrazole (234 mg, 1.45 mmol, 1.2 eq) in dioxane (8.0 mL) was added t-BuONa (582 mg, 6.05 mmol, 5.0 eq) and fBuXPhos Pd G3 (96.2 mg, 121 μmol, 0.1 eq) under a N₂ atmosphere. The mixture was stirred at 100° C. for 12 h. LCMS indicated that the starting material was completely consumed, and the desired mass was detected. The reaction mixture was allowed to cool to room temperature, poured into H₂O (10 mL) and washed with petroleum ether (3.0 mL×5). The aqueous layer was treated with HCl (1 M aqueous) to adjust pH 4 and extracted with EtOAc (2.0 mL×5). The combined organic layers dried over Na₂SO₄, filtered and concentrated under vacuum to give the crude product 2-methyl-5-((1-methyl-1H-pyrazol-4-yl)amino)benzoic acid (0.25 g) as a brown oil.

### Step 2: (R)—N-(1-(3-(5-Formylthiophen-2-yl)phenyl)ethyl)-2-methyl-5-((1-methyl-1H-pyrazol-4-yl)amino)benzamide (248A-2)

To a solution of 2-methyl-5-((1-methyl-1H-pyrazol-4-yl)amino)benzoic acid (198 mg, 856 μmol, 1.1 eq) and (R)-5-(3-(1-aminoethyl)phenyl)thiophene-2-carbaldehyde (180 mg, 778 μmol, 1.0 eq) in DCM (6.0 mL) were added TEA (236 mg, 2.33 mmol, 325 μL, 3.0 eq), EDCI (179 mg, 934 μmol, 1.2 eq) and HOBt (126 mg, 934 μmol, 1.2 eq). The mixture was stirred at 20° C. for 16 h. LCMS indicated that the starting material was completely consumed, and the desired mass was detected. The reaction mixture was poured into H₂O (5.0 mL) and extracted with DCM (5 mL×2). The combined organic layers were dried over Na₂SO₄, filtered, and concentrated under vacuum to give a residue which was purified by preparative TLC (DCM/MeOH=10/1, R_{f}=0.6). (R)—N-(1-(3-(5-Formylthiophen-2-yl)phenyl)ethyl)-2-methyl-5-((1-methyl-1H-pyrazol-4-yl)amino)benzamide (75.0 mg, 169 μmol, 22% yield) was obtained as a yellow solid. M+H⁺=445.1 (LCMS).

### Step 3: N—((R)-1-(3-(5-((((1S,3R)-3-Hydroxycyclopentyl)amino)methyl)thiophen-2-yl)phenyl)ethyl)-2-methyl-5-((1-methyl-1H-pyrazol-4-yl)amino)benzamide (Compound 137)

To a solution of (1R,3S)-3-aminocyclopentanol (25.5 mg, 186 μmol, 25 μL, 1.1 eq) in MeOH (3.0 mL) was added TEA (10.0 μL), followed by the addition of (R)—N-(1-(3-(5-formylthiophen-2-yl)phenyl)ethyl)-2-methyl-5-((1-methyl-1H-pyrazol-4-yl)amino)benzamide (75.0 mg, 169 μmol, 1.0 eq). The resulting mixture was adjusted to pH 6 with a small amount of AcOH. The mixture was stirred at 20° C. for 30 min, then NaBH₃CN (21.2 mg, 337 μmol, 2.0 eq) was added. The result mixture was stirred at 20° C. for another 15 h. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give a residue which was purified by preparative HPLC (Phenomenex Gemini C18 column (80× 30 mm, 3 μm); flow rate: 25 mL/min; gradient: 15%-35% B over 8 min; mobile phase A: 0.1% aqueous TFA, mobile phase B: acetonitrile). N—((R)-1-(3-(5-((((1S,3R)-3-Hydroxycyclopentyl)amino)methyl)thiophen-2-yl)phenyl)ethyl)-2-methyl-5-((1-methyl-1H-pyrazol-4-yl)amino)benzamide (7.00 mg, 13.2 μmol, 8% yield, TFA salt) was obtained as a white solid. M+H⁺=530.2 (LCMS); ¹HNMR (400 MHZ, CD₃OD) δ 7.69-7.65 (m, 1H), 7.58-7.51 (m, 2H), 7.45-7.35 (m, 4H), 7.29-7.25 (m, 1H), 7.05-6.98 (m, 1H), 6.85-6.74 (m, 2H), 5.26-5.13 (m, 1H), 4.55-4.39 (m, 2H), 4.35-4.33 (m, 1H), 3.88-3.72 (m, 3H), 3.71-3.68 (m, 1H), 2.27-2.18 (m, 5H), 1.97-1.84 (m, 4H), 1.61-1.41 (m, 3H).

### Example 249: 5-((1H-Pyrazol-4-yl)amino)-N—((R)-1-(3-(5-((((1S,3R)-3-hydroxycyclopentyl)amino)methyl)thiophen-2-yl)phenyl)ethyl)-2-methylbenzamide (Compound 140)

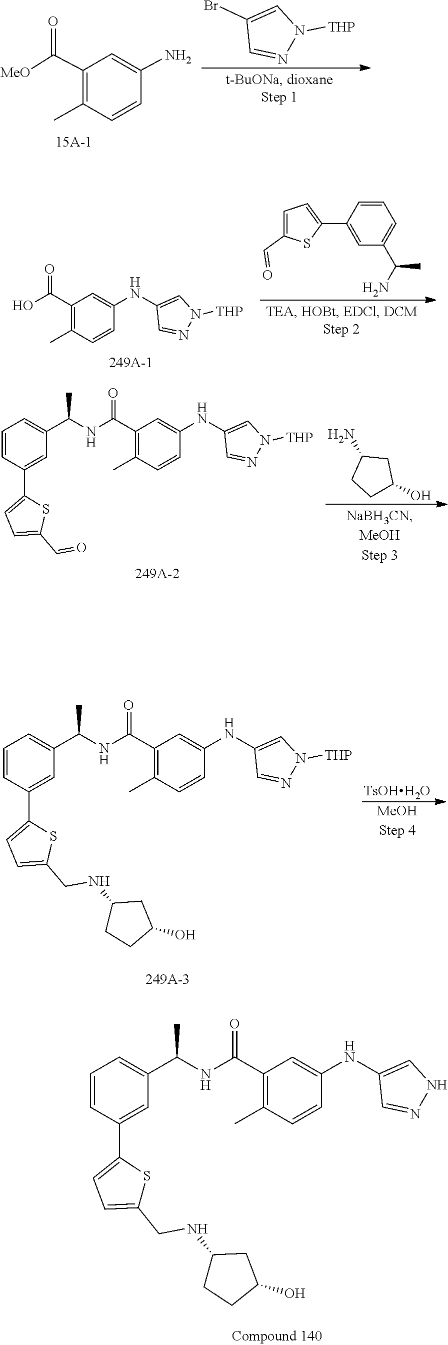

### Step 1: 2-Methyl-5-((1-(tetrahydro-2H-pyran-2-yl)-1H-pyrazol-4-yl)amino)benzoic acid (249A-1)

To a mixture of methyl 5-amino-2-methylbenzoate (200 mg, 1.21 mmol, 1.0 eq) and 4-bromo-1-(tetrahydro-2H-pyran-2-yl)-1H-pyrazole (336 mg, 1.45 mmol, 1.2 eq) in anhydrous dioxane (8.0 mL) were added t-BuONa (582 mg, 6.05 mmol, 5.0 eq) and/BuXPhos Pd G3 (96.2 mg, 121 μmol, 0.1 eq) degassed and purged with N₂ three times. The mixture was stirred at 100° C. for 16 h. LCMS indicated that the starting material was completely consumed, and the desired mass was detected. The reaction mixture was allowed to cool to room temperature, poured into H₂O (5.0 mL) and extracted with DCM (3.0 mL×3). The combined organic layers were dried over Na₂SO₄, filtered, and concentrated under vacuum to give 2-methyl-5-((1-(tetrahydro-2H-pyran-2-yl)-1H-pyrazol-4-yl)amino)benzoic acid (210 mg, 697 μmol, 58% yield) as a yellow solid. M+H⁺=302.3 (LCMS).

### Step 2: N—((R)-1-(3-(5-Formylthiophen-2-yl)phenyl)ethyl)-2-methyl-5-((1-(tetrahydro-2H-pyran-2-yl)-1H-pyrazol-4-yl)amino)benzamide (249A-2)

To a solution of 2-methyl-5-((1-(tetrahydro-2H-pyran-2-yl)-1H-pyrazol-4-yl)amino)benzoic acid (72.6 mg, 241 μmol, 1.1 eq) and (R)-5-(3-(1-aminoethyl)phenyl)thiophene-2-carbaldehyde (50.7 mg, 219 μmol, 1.0 eq) in DCM (3.0 mL) were added TEA (66.5 mg, 657 μmol, 91.5 μL, 3.0 eq), EDCI (50.4 mg, 263 μmol, 1.2 eq) and HOBt (35.5 mg, 263 μmol, 1.2 eq). The mixture was stirred at 20° C. for 16 h. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give a residue which was purified by preparative TLC (DCM/MeOH=10/1, R_{f}=0.7). N—((R)-1-(3-(5-Formylthiophen-2-yl)phenyl)ethyl)-2-methyl-5-((1-(tetrahydro-2H-pyran-2-yl)-1H-pyrazol-4-yl)amino)benzamide (85.0 mg, 165 μmol, 75% yield) was obtained as a yellow solid. M+H⁺=515.4 (LCMS).

### Step 3: N—((R)-1-(3-(5-((((1S,3R)-3-Hydroxycyclopentyl)amino)methyl)thiophen-2-yl)phenyl)ethyl)-2-methyl-5-((1-(tetrahydro-2H-pyran-2-yl)-1H-pyrazol-4-yl)amino)benzamide (249A-3)

To a stirred solution of N—((R)-1-(3-(5-formylthiophen-2-yl)phenyl)ethyl)-2-methyl-5-((1-(tetrahydro-2H-pyran-2-yl)-1H-pyrazol-4-yl)amino)benzamide (85.0 mg, 165 μmol, 1.0 eq) and (1R,3S)-3-aminocyclopentanol (25.0 mg, 182 μmol, 1.1 eq) in MeOH (3.0 mL). The resulting mixture was adjusted to pH 6 with a small amount of AcOH. The mixture was stirred at 20° C. for 30 min, then mixture NaBH₃CN (20.8 mg, 330 μmol, 2.0 eq) was added. The mixture was stirred at 20° C. for 16 h. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 40%-70% B over 8 min; mobile phase A: 10 mM aqueous NH₄HCO₃, mobile phase B: acetonitrile). N—((R)-1-(3-(5-((((1S,3R)-3-hydroxycyclopentyl)amino)methyl)thiophen-2-yl)phenyl)ethyl)-2-methyl-5-((1-(tetrahydro-2H-pyran-2-yl)-1H-pyrazol-4-yl)amino)benzamide (20.0 mg, 33.4 μmol, 20% yield) was obtained as a yellow solid. M+H⁺=600.3 (LCMS).

### Step 4: 5-((1H-Pyrazol-4-yl)amino)-N—((R)-1-(3-(5-((((1S,3R)-3-hydroxycyclopentyl)amino)methyl)thiophen-2-yl)phenyl)ethyl)-2-methylbenzamide (Compound 140)

To a mixture of N—((R)-1-(3-(5-((((1S,3R)-3-hydroxycyclopentyl)amino)methyl)thiophen-2-yl)phenyl)ethyl)-2-methyl-5-((1-(tetrahydro-2H-pyran-2-yl)-1H-pyrazol-4-yl)amino)benzamide (20.0 mg, 33.4 μmol, 1.0 eq) in MeOH (2.0 mL) was added TsOH·H₂O (19.0 mg, 100 μmol, 3.0 eq). The mixture was stirred at 20° C. for 16 h. LCMS indicated that the starting material was completely consumed, and the desired mass was detected. The mixture was concentrated under vacuum to give a residue which was purified by preparative HPLC (Phenomenex Luna C18 column (150×30 mm, 5 μm); flow rate: 25 mL/min; gradient: 20%-50% B over 8 min; mobile phase A: 0.1% aqueous TFA, mobile phase B: acetonitrile). 5-((1H-Pyrazol-4-yl)amino)-N—((R)-1-(3-(5-((((1S,3R)-3-hydroxycyclopentyl)amino)methyl)thiophen-2-yl)phenyl)ethyl)-2-methylbenzamide (4.12 mg, 6.54 μmol, 20% yield, TFA salt) was obtained as a white solid. M+H⁺=516.1 (LCMS); ¹H NMR (400 MHZ, CD₃OD) δ 7.65 (s, 1H), 7.57-7.52 (m, 3H), 7.43-7.35 (m, 3H), 7.27 (d, J=3.6 Hz, 1H), 7.02 (d, J=8.3 Hz, 1H), 6.84-6.76 (m, 2H), 5.19 (q, J=7.0 Hz, 1H), 4.47 (s, 2H), 4.34 (quin, J=4.1 Hz, 1H), 3.74-3.66 (m, 1H), 2.30-2.23 (m, 1H), 2.22 (s, 3H), 2.20-2.14 (m, 1H), 2.04-1.79 (m, 4H), 1.53 (d, J=7.0 Hz, 3H).

### Example 250: N—((R)-1-(3-(5-((((1S,3R)-3-Hydroxycyclopentyl)amino)methyl)thiophen-2-yl)phenyl)ethyl)-5-methyl-1H-indazole-6-carboxamide (Compound 146)

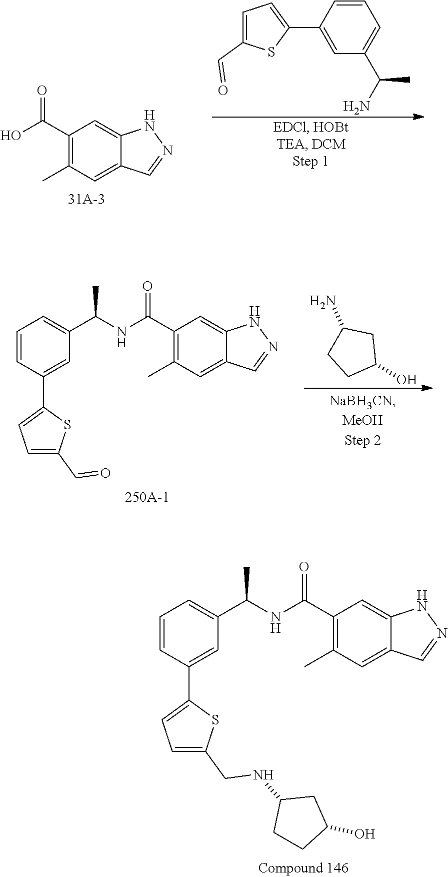

### Step 1: (R)—N-(1-(3-(5-Formylthiophen-2-yl)phenyl)ethyl)-5-methyl-1H-indazole-6-carboxamide (250A-1)

To a solution of 5-methyl-1H-indazole-6-carboxylic acid (70.0 mg, 397 μmol, 1.0 eq) and (R)-5-(3-(1-aminoethyl)phenyl)thiophene-2-carbaldehyde (91.9 mg, 397 μmol, 20.0 μL, 1.0 eq) in DCM (2.0 mL) were added TEA (80.4 mg, 795 μmol, 111 μL, 2.0 eq), EDCI (91.4 mg, 477 μmol, 1.2 eq) and HOBt (64.4 mg, 477 μmol, 1.2 eq). The mixture was stirred at 20° C. for 3 h. LCMS indicated that the starting material was completely consumed, and the desired mass was detected. The reaction mixture was poured into H₂O (5.0 mL) and extracted with DCM (4.0 mL×3). The combined organic layers were dried over Na₂SO₄, filtered, and concentrated under vacuum to give the crude product (R)—N-(1-(3-(5-formylthiophen-2-yl)phenyl)ethyl)-5-methyl-1H-indazole-6-carboxamide (130 mg) as a brown oil. M+H⁺=390.2 (LCMS).

### Step 2: N—((R)-1-(3-(5-((((1S,3R)-3-Hydroxycyclopentyl)amino)methyl)thiophen-2-yl)phenyl)ethyl)-5-methyl-1H-indazole-6-carboxamide (Compound 146)

To a solution of (1R,3S)-3-aminocyclopentanol (50.5 mg, 367 μmol, 1.1 eq, HCl salt) in MeOH (2.0 mL) was added TEA (30.0 μL), followed by the addition of (R)—N-(1-(3-(5-formylthiophen-2-yl)phenyl)ethyl)-5-methyl-1H-indazole-6-carboxamide (130 mg, 334 μmol, 1.0 eq). The resulting mixture was adjusted to pH 6 with a small amount of AcOH. The mixture was stirred at 20° C. for 30 min, and then NaBH₃CN (62.9 mg, 1.00 mmol, 3.0 eq) was added. The resulting reaction mixture was stirred at 20° C. for another 15 h. LCMS indicated that the starting material was completely consumed, and the desired mass was detected. The reaction mixture was poured into H₂O (4.0 mL) and extracted with EtOAc (4.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 5%-30% B over 8 min; mobile phase A: 0.04% aqueous HCl, mobile phase B: acetonitrile). N—((R)-1-(3-(5-((((1S,3R)-3-Hydroxycyclopentyl)amino)methyl)thiophen-2-yl)phenyl)ethyl)-5-methyl-1H-indazole-6-carboxamide (14.1 mg, 29.0 μmol, 9% yield) was obtained as a white solid. M+H⁺=475.1 (LCMS); ¹H NMR (400 MHz, CD₃OD) δ 8.27 (br s, 1H), 7.74 (s, 2H), 7.70 (s, 1H), 7.62 (s, 1H), 7.42 (dd, J=4.3, 7.8 Hz, 3H), 7.30 (d, J=3.7 Hz, 1H), 5.28 (q, J=7.0 Hz, 1H), 4.47 (s, 2H), 4.40-4.28 (m, 1H), 3.77-3.64 (m, 1H), 2.42 (s, 3H), 2.08-1.93 (m, 2H), 1.86 (br dd, J=3.7, 8.0 Hz, 4H), 1.60 (d, J=7.0 Hz, 3H).

### Example 251: 5-(2-Aminoethoxy)-2-methyl-N-(1-(naphthalen-1-yl)cyclopropyl)benzamide (Compound 151)

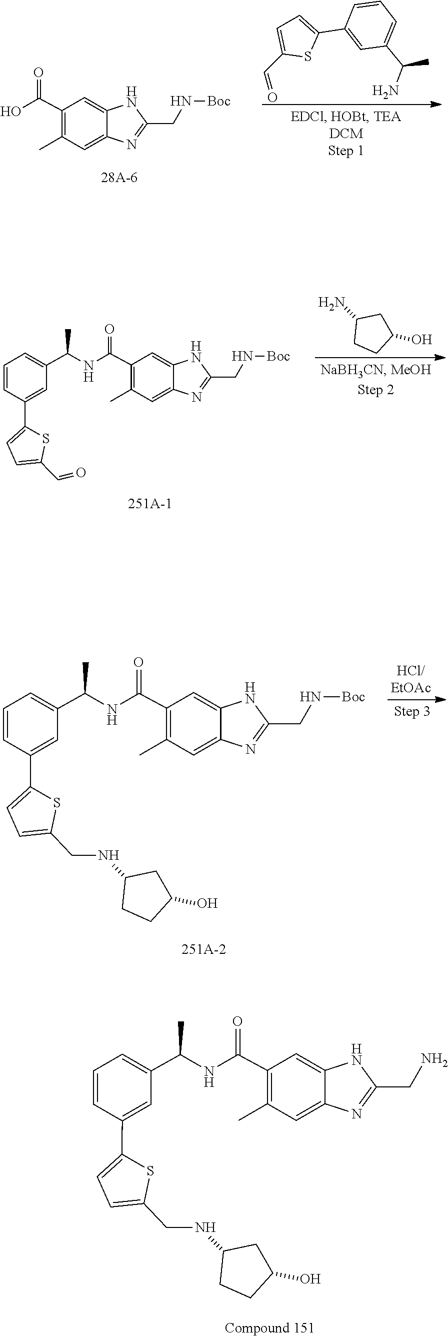 [SMILES: Cc1cc2nc(CNC(=O)OC(C)(C)C)[nH]c2cc1C(=O)O ; C[C@@H](N)c1cccc(-c2ccc(C=O)s2)c1 ; Cc1cc2nc(CNC(=O)OC(C)(C)C)[nH]c2cc1C(=O)N[C@H](C)c1cccc(-c2ccc(C=O)s2)c1 ; N[C@H]1CC[C@@H](O)C1 ; Cc1cc2nc(CNC(=O)OC(C)(C)C)[nH]c2cc1C(=O)N[C@H](C)c1cccc(-c2ccc(CN[C@H]3CC[C@@H](O)C3)s2)c1 ; Cc1cc2nc(CN)[nH]c2cc1C(=O)N[C@H](C)c1cccc(-c2ccc(CN[C@H]3CC[C@@H](O)C3)s2)c1]

### Step 1: (R)-tert-Butyl((6-((1-(3-(5-formylthiophen-2-yl)phenyl)ethyl)carbamoyl)-5-methyl-1H-benzo[d]imidazol-2-yl)methyl)carbamat (251A-1)

To a solution of 2-(((tert-butoxycarbonyl)amino)methyl)-5-methyl-1H-benzo[d]imidazole-6-carboxylic acid (100 mg, 328 μmol, 1.0 eq) and (R)-5-(3-(1-aminoethyl)phenyl)thiophene-2-carbaldehyde (75.8 mg, 328 μmol, 1.0 eq) in DCM (5.0 mL) were added TEA (66.3 mg, 655 μmol, 91.2 μL, 2.0 eq), EDCI (75.3 mg, 393 μmol, 1.2 eq) and HOBt (23.1 mg, 393 μmol, 1.2 eq). The mixture was stirred at 25° C. for 1 h. LCMS indicated that the starting material was completely consumed, and the desired mass was detected. The reaction mixture was poured into H₂O (5.0 mL) and extracted with DCM (3.0 mL×5). The combined organic layers were dried over Na₂SO₄, filtered, and concentrated under vacuum to give a residue which was purified by flash silica gel chromatography using a gradient of EtOAc/petroleum ether from 1/100 to 1/2. (R)-tert-Butyl((6-((1-(3-(5-formylthiophen-2-yl)phenyl)ethyl)carbamoyl)-5-methyl-1H-benzo[d]imidazole-2-yl)methyl)carbamate (70 mg, 135 μmol, 41% yield) was obtained as a white solid. M+H⁺=519.3 (LCMS).

### Step 2: tert-Butyl((6-(((R)-1-(3-(5-((((1S,3R)-3-hydroxycyclopentyl)amino)methyl)thiophen-2-yl)phenyl)ethyl)carbamoyl)-5-methyl-1H-benzo[d]imidazol-2-yl)methyl) carbamate (251A-2)

To a solution of (1R,3S)-3-aminocyclopentanol (47.8 mg, 347 μmol, 3.0 eq) in MeOH (5.0 mL) was added TEA (10.0 μL), followed by the addition of (R)-tert-butyl((6-((1-(3-(5-formylthiophen-2-yl)phenyl)ethyl)carbamoyl)-5-methyl-1H-benzo[d]imidazol-2-yl)methyl) carbamate (60.0 mg, 116 μmol, 1.0 eq). The resulting mixture was adjusted to pH 6 with a small amount of AcOH. The mixture was stirred at 20° C. for 30 min, then NaBH₃CN (21.8 mg, 347 μmol, 3.0 eq) was added. The reaction mixture was stirred at 20° C. for another 16 h. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give the crude product tert-butyl((6-(((R)-1-(3-(5-((((1S,3R)-3-hydroxycyclopentyl)amino)methyl)thiophen-2-yl)phenyl)ethyl)carbamoyl)-5-methyl-1H-benzo[d]imidazol-2-yl)methyl)carbamate (70.0 mg). M+H⁺=604.4 (LCMS).

### Step 3: 2-(Aminomethyl)-N—((R)-1-(3-(5-((((1S,3R)-3-hydroxycyclopentyl)amino)methyl)thiophen-2-yl)phenyl)ethyl)-5-methyl-1H-benzo[d]imidazole-6-carboxamide (Compound 151)

To a stirred solution of tert-butyl((6-(((R)-1-(3-(5-((((1S,3R)-3-hydroxycyclopentyl)amino)methyl)thiophen-2-yl)phenyl)ethyl)carbamoyl)-5-methyl-1H-benzo[d]imidazol-2-yl)methyl) carbamate (50.0 mg, 82.8 μmol, 1.0 eq) in EtOAc (3.0 mL) was added HCl/EtOAc (4 M, 82.8 μL, 4.0 eq). The mixture was stirred at 25° C. for 1 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 1%-20% B over 8 min; mobile phase A: 0.04% aqueous HCl, mobile phase B: acetonitrile) to give 2-(aminomethyl)-N—((R)-1-(3-(5-((((1S,3R)-3-hydroxycyclopentyl)amino)methyl)thiophen-2-yl)phenyl)ethyl)-5-methyl-1H-benzo[d]imidazole-6-carboxamide (5.50 mg, 9.96 μmol, 12% yield, HCl salt) as a white solid. M+H⁺=504.1 (LCMS); ¹H NMR (400 MHZ, CD₃OD) δ 7.81 (s, 1H), 7.74 (s, 1H), 7.69 (s, 1H), 7.58 (dd, J=2.4, 6.2 Hz, 1H), 7.43 (t, J=4.8 Hz, 3H), 7.31 (d, J=3.7 Hz, 1H), 5.27 (d, J=7.1 Hz, 1H), 4.69 (s, 2H), 4.48 (s, 2H), 4.37-4.31 (m, 1H), 3.71 (br t, J=6.1 Hz, 1H), 2.50 (s, 3H), 2.34-2.13 (m, 2H), 2.07-1.94 (m, 1H), 1.91-1.82 (m, 3H), 1.60 (d, J=7.1 Hz, 3H).

### Example 252: N—((R)-1-(3-(5-((((1S,3R)-3-Hydroxycyclopentyl)amino)methyl)thiophen-2-yl)phenyl)ethyl)-6-methylquinoline-7-carboxamide (Compound 154)

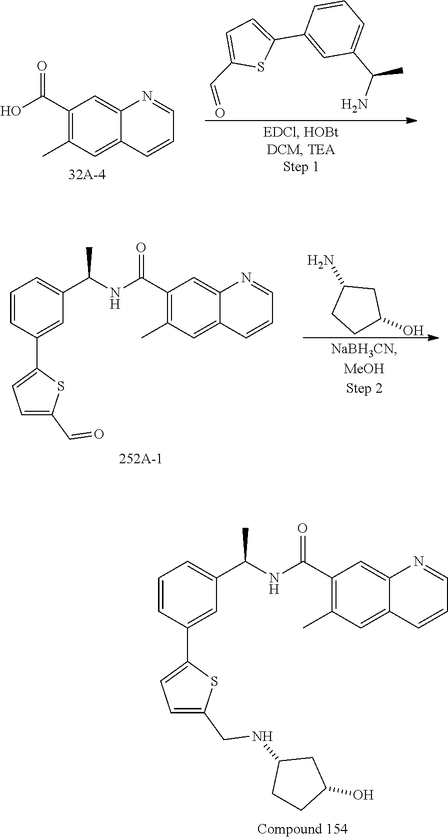

### Step 1: (R)—N-(1-(3-(5-formylthiophen-2-yl)phenyl)ethyl)-6-methylquinoline-7-carboxamide (252A-1)

To a solution of 6-methylquinoline-7-carboxylic acid (70.0 mg, 374 μmol, 1.0 eq) in DCM (5 mL) were added (R)-5-(3-(1-aminoethyl)phenyl)thiophene-2-carbaldehyde (110 mg, 411 μmol, 83 μL, 1.1 eq, HCl salt), TEA (75.7 mg, 748 μmol, 104 μL, 2.0 eq), EDCI (108 mg, 561 μmol, 1.5 eq) and HOBt (75.8 mg, 561 μmol, 1.5 eq). The mixture was stirred at 20° C. for 16 h. TLC indicated that the starting material was completely consumed. The reaction mixture was poured into H₂O (10 mL) and extracted with DCM (10 mL×2). The combined organic layers were dried over Na₂SO₄, filtered, and concentrated under vacuum to give a residue which was purified by flash silica gel chromatography using a gradient of EtOAc/petroleum ether from 0/1 to 1/1. (R)—N-(1-(3-(5-Dormylthiophen-2-yl)phenyl)ethyl)-6-methylquinoline-7-carboxamide (130 mg, 325 μmol, 87% yield) was obtained as a yellow oil. ¹H NMR (400 MHZ, CD₃OD) δ 9.93-9.83 (m, 1H), 8.88-8.78 (m, 1H), 8.32 (d, J=8.1 Hz, 1H), 8.05-7.99 (m, 1H), 7.93 (d, J=4.0 Hz, 1H), 7.88-7.83 (m, 1H), 7.83-7.80 (m, 1H), 7.75-7.68 (m, 1H), 7.64-7.61 (m, 1H), 7.59-7.48 (m, 3H), 5.33 (q, J=7.1 Hz, 1H), 2.57-2.49 (m, 3H), 1.66-1.59 (m, 3H).

### Step 2: N—((R)-1-(3-(5-((((1S,3R)-3-hydroxycyclopentyl)amino)methyl)thiophen-2-yl)phenyl)ethyl)-6-methylquinoline-7-carboxamide (Compound 154)

To a solution of (1R,3S)-3-aminocyclopentanol (179 mg, 1.30 mmol, 4.0 eq, HCl salt) in MeOH (5.0 mL) was added TEA (40.0 μL), followed by the addition of (R)—N-(1-(3-(5-dormylthiophen-2-yl)phenyl)ethyl)-6-methylquinoline-7-carboxamide (130 mg, 325 μmol, 1.0 eq). The resulting mixture was adjusted to pH 6 with a small amount of AcOH. The mixture was stirred at 20° C. for 30 min, then NaBH₃CN (134 mg, 2.13 mmol, 2.0 eq) was added. The reaction mixture was stirred at 20° C. for another 16 h. LCMS indicated that the starting material was completely consumed, and the desired mass was detected. The reaction mixture was poured into H₂O (5.0 mL) and extracted with EtOAc (5.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 1%-25% B over 8 min; mobile phase A: 0.04% aqueous HCl, mobile phase B: acetonitrile). N—((R)-1-(3-(5-((((1S,3R)-3-Hydroxycyclopentyl)amino)methyl)thiophen-2-yl)phenyl)ethyl)-6-methylquinoline-7-carboxamide (39.0 mg, 80.3 μmol, 25% yield, HCl salt) was obtained as a white solid. M+H⁺=486.1 (LCMS); HNMR: 1H NMR (400 MHZ, CD₃OD) δ 9.23-9.19 (m, 1H), 9.14 (d, J=8.4 Hz, 1H), 8.22 (s, 1H), 8.18 (s, 1H), 8.12 (dd, J=5.4, 8.4 Hz, 1H), 7.76 (s, 1H), 7.60 (dt, J=1.8, 4.3 Hz, 1H), 7.50-7.39 (m, 2H), 7.31 (d, J=3.6 Hz, 1H), 7.30 (m, 1H), 5.38-5.28 (m, 1H), 4.50-4.44 (m, 2H), 4.38-4.30 (m, 1H), 3.76-3.64 (m, 1H), 2.59 (s, 3H), 2.32-2.13 (m, 2H), 2.07-1.95 (m, 1H), 1.92-1.81 (m, 3H), 1.64 (d, J=7.1 Hz, 3H).

### Example 253: 5-(2-Aminoethoxy)-2-methyl-N-(1-(naphthalen-1-yl)cyclopropyl)benzamide (Compound 143)

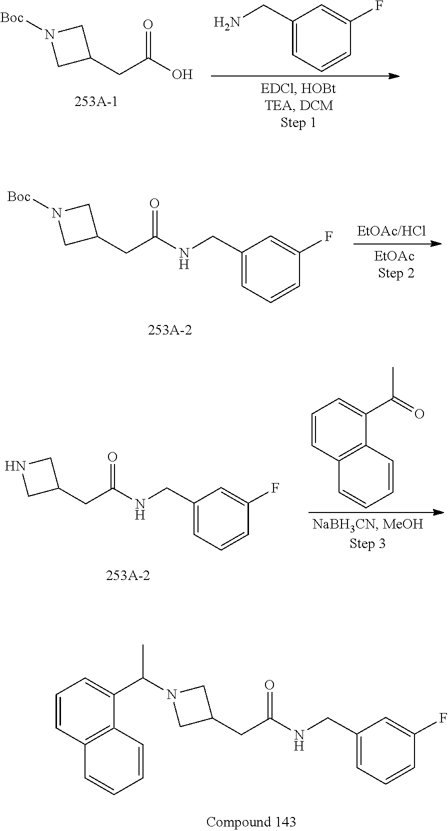 [SMILES: CC(C)(C)OC(=O)N1CC(CC(=O)O)C1 ; NCc1cccc(F)c1 ; CC(C)(C)OC(=O)N1CC(CC(=O)NCc2cccc(F)c2)C1 ; O=C(CC1CNC1)NCc1cccc(F)c1 ; CC(=O)c1cccc2ccccc12 ; CC(c1cccc2ccccc12)N1CC(CC(=O)NCc2cccc(F)c2)C1]

### Step 1: tert-Butyl 3-(2-((3-fluorobenzyl)amino)-2-oxoethyl)azetidine-1-carboxylate (253A-2)

To a solution of 2-(1-(tert-butoxycarbonyl)azetidin-3-yl) acetic acid (500 mg, 2.32 mmol, 1.0 eq) in DCM (15 mL) were added (3-fluorophenyl) methanamine (436 mg, 3.48 mmol, 396 μL, 1.5 eq), TEA (705 mg, 6.97 mmol, 970 μL, 3.0 eq), EDCI (668 mg, 3.48 mmol, 1.5 eq) and HOBt (471 mg, 3.48 mmol, 1.5 eq). The resulting mixture was stirred at 20° C. for 2 h. LCMS indicated that the starting material was completely consumed, and the desired mass was detected. The reaction mixture was poured into H₂O (30 mL) and extracted with DCM (20 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. tert-Butyl 3-(2-((3-fluorobenzyl)amino)-2-oxo ethyl)azetidine-1-carboxylate (700 mg, 2.17 mmol, 93% yield) was obtained as a colorless oil. M+H⁺=323.3 (LCMS); ¹H NMR (400 MHZ, CDCl₃) δ 7.36-7.28 (m, 1H), 7.07-6.92 (m, 3H), 4.43 (br d, J=5.9 Hz, 2H), 4.21-4.01 (m, 2H), 3.72-3.49 (m, 2H), 3.07-2.87 (m, 1H), 2.55 (br d, J=7.9 Hz, 2H), 1.47-1.40 (m, 9H).

### Step 2: 2-(Azetidin-3-yl)-N-(3-fluorobenzyl) acetamide (253A-3)

To a solution of tert-butyl 3-(2-((3-fluorobenzyl)amino)-2-oxoethyl)azetidine-1-carboxylate (300 mg, 936 μmol, 1 eq) in EtOAc (10 mL) was added HCl/EtOAc (4 M, 30.0 mL) at 0° C. Then the mixture was stirred at 25° C. for 1 h. LCMS indicated that the starting material was completely consumed, and the desired mass was detected. The mixture was concentrated under vacuum at 30° C. to give the crude product 2-(azetidin-3-yl)-N-(3-fluorobenzyl) acetamide (300 mg, HCl salt) as a yellow oil, which was used in the next step without any further purification. M+H⁺=223.3 (LCMS).

### Step 3: N-(3-Fluorobenzyl)-2-(1-(1-(naphthalen-1-yl)ethyl)azetidin-3-yl)acetamide (Compound 143)

To a solution of 2-(azetidin-3-yl)-N-(3-fluorobenzyl) acetamide (300 mg, 1.16 mmol, 2.63 eq, HCl salt) in MeOH (15 mL) was added TEA (50.0 μL), followed by the addition of 1-(1-naphthyl)ethanone (150 mg, 881 μmol, 134 μL, 2.0 eq). The resulting mixture was adjusted to pH 6 with a small amount of AcOH. The mixture was stirred at 20° C. for 30 min, then NaBH₃CN (83.1 mg, 1.32 mmol, 3.0 eq) was added. The resulting reaction mixture was stirred at 20° C. for another 16 h. LCMS indicated that the starting material was completely consumed, and the desired mass was detected. The reaction mixture was poured into H₂O (30 mL) and extracted with EtOAc (20 mL×3). The combined organic layers were dried over Na₂SO₄, filtered, and concentrated under vacuum to give a residue which was purified by preparative HPLC (Phenomenex C18 column (75×30 mm, 3 μm); flow rate: 25 mL/min; gradient: 30%-70% B over 8 min; mobile phase A: 10 mM aqueous NH₄HCO₃; mobile phase B: acetonitrile). N-(3-Fluorobenzyl)-2-(1-(1-(naphthalen-1-yl)ethyl)azetidin-3-yl) acetamide (7.82 mg, 20.8 μmol, 5% yield) was obtained as a yellow gum. ¹H NMR (400 MHZ, CD₃OD) δ 8.20 (br d, J=8.3 Hz, 1H), 7.89-7.84 (m, 1H), 7.75 (d, J=8.3 Hz, 1H), 7.58-7.41 (m, 4H), 7.29 (dt, J=6.0, 7.8 Hz, 1H), 7.06 (d, J=7.5 Hz, 1H), 7.02-6.89 (m, 2H), 4.35-4.26 (m, 3H), 3.66-3.57 (m, 1H), 3.50-3.40 (m, 1H), 3.08 (t, J=7.1 Hz, 1H), 2.90-2.78 (m, 2H), 2.52 (d, J=7.0 Hz, 2H), 1.33 (d, J=6.6 Hz, 3H).

### Example 254: N-(3-Fluorobenzyl)-1-(1-(naphthalen-1-yl)ethyl) pyrrolidine-3-carboxamide

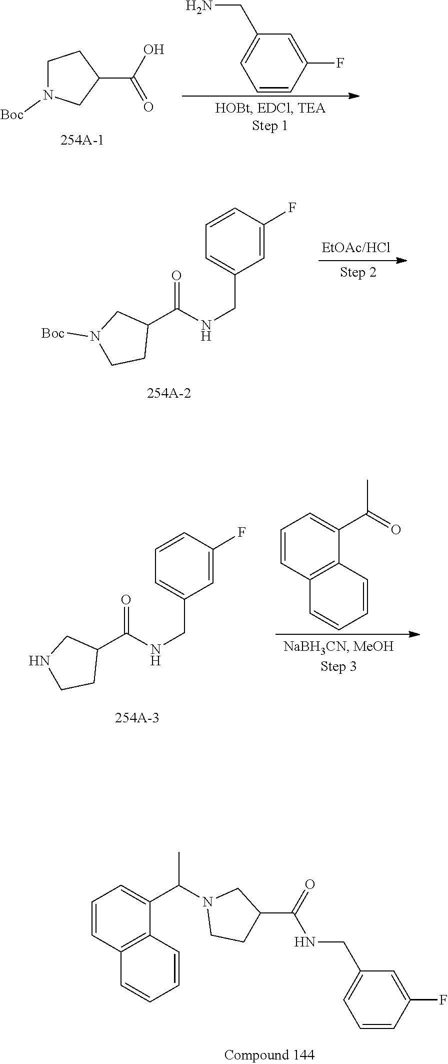

### Step 1: tert-Butyl 3-((3-fluorobenzyl)carbamoyl) pyrrolidine-1-carboxylate (254A-2)

To a solution of 1-(tert-butoxycarbonyl)pyrrolidine-3-carboxylic acid (1.00 g, 4.65 mmol, 1.0 eq) and (3-fluorophenyl) methanamine (581 mg, 4.65 mmol, 0.53 mL, 1.0 eq) in DCM (15 mL) were added TEA (940 mg, 9.29 mmol, 1.30 mL, 2.0 eq), EDCI (1.07 g, 5.58 mmol, 1.2 eq) and HOBt (753 mg, 5.58 mmol, 1.2 eq). The mixture was stirred at 20° C. for 16 h. TLC indicated that the starting material was completely consumed. The reaction mixture was poured into H₂O (15 mL) and extracted with DCM (5.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. tert-Butyl 3-((3-fluorobenzyl)carbamoyl) pyrrolidine-1-carboxylate (600 mg, 1.86 mmol, 40% yield) was obtained as a white solid. M−56+H⁺=267.2 (LCMS); ¹H NMR (400 MHZ, DMSO-d₆) δ 8.52 (br s, 1H), 7.36 (dt, J=6.4, 7.7 Hz, 1H), 7.14-6.94 (m, 3H), 4.29 (d, J=6.0 Hz, 2H), 3.45 (dd, J=8.0, 10.5 Hz, 1H), 3.40-3.33 (m, 1H), 3.31-3.26 (m, 1H), 3.25-3.15 (m, 1H), 3.06-2.90 (m, 1H), 2.05-1.89 (m, 2H), 1.39 (s, 9H).

### Step 2: N-(3-Fluorobenzyl)pyrrolidine-3-carboxamide (254A-3)

To a stirred solution of tert-butyl 3-((3-fluorobenzyl)carbamoyl)pyrrolidine-1-carboxylate (200 mg, 434 μmol, 1.0 eq) in EtOAc (1.0 mL) was added HCl/EtOAc (4 M, 10 mL). The mixture was stirred at 20° C. for 1 h. LCMS indicated that the starting material was completely consumed, and the desired mass was detected. The mixture was concentrated under vacuum at 30° C. to give the crude product N-(3-fluorobenzyl)pyrrolidine-3-carboxamide (200 mg), which was used in the next step without any further purification. M+H⁺=223.1 (LCMS).

### Step 3: N-(3-Fluorobenzyl)-1-(1-(naphthalen-1-yl)ethyl)pyrrolidine-3-carboxamide (Compound 144)

To a solution of N-(3-fluorobenzyl)pyrrolidine-3-carboxamide (200 mg, 773 μmol, 1.0 eq, HCl salt) in MeOH (10 mL) was added TEA (10.0 μL), followed by the addition of 1-(naphthalen-1-yl)ethanone (132 mg, 773 μmol, 120 μL 1.0 eq). The resulting mixture was adjusted to pH 6 with a small amount of AcOH. The mixture was stirred at 20° C. for 30 min, then NaBH₃CN (146 mg, 2.32 mmol, 3.0 eq) was added. The resulting mixture was stirred at 20° C. for another 16 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by preparative HPLC (Waters Xbridge Prep OBD C18 column (150×40 mm, 10 μm); flow rate: 25 mL/min; gradient: 45%-75% B over 8 min; mobile phase A: 10 mM aqueous NH₄HCO₃, mobile phase B: acetonitrile). N-(3-Fluorobenzyl)-1-(1-(naphthalen-1-yl)ethyl)pyrrolidine-3-carboxamide (87.4 mg, 232 μmol, 30% yield) was obtained as a colorless gum. M+H⁺=377.3 (LCMS); ¹H NMR (400 MHZ, CD₃OD) δ 8.39 (br d, J=7.2 Hz, 1H), 7.89-7.82 (m, 1H), 7.75 (d, J=8.1 Hz, 1H), 7.62 (dd, J=3.8, 6.7 Hz, 1H), 7.50-7.38 (m, 3H), 7.34-7.22 (m, 1H), 7.11-6.84 (m, 3H), 4.39-4.30 (m, 1H), 4.27 (d, J=8.7 Hz, 1H), 4.23-4.10 (m, 1H), 3.06-2.87 (m, 2H), 2.83-2.53 (m, 3H), 2.22-1.95 (m, 2H), 1.59-1.45 (m, 3H).

### Example 255: (1R,5S,6r)-N-(3-Fluorobenzyl)-3-(1-(naphthalen-1-yl)ethyl)-3-azabicyclo[3.1.0]hexane-6-carboxamide (Compound 149)

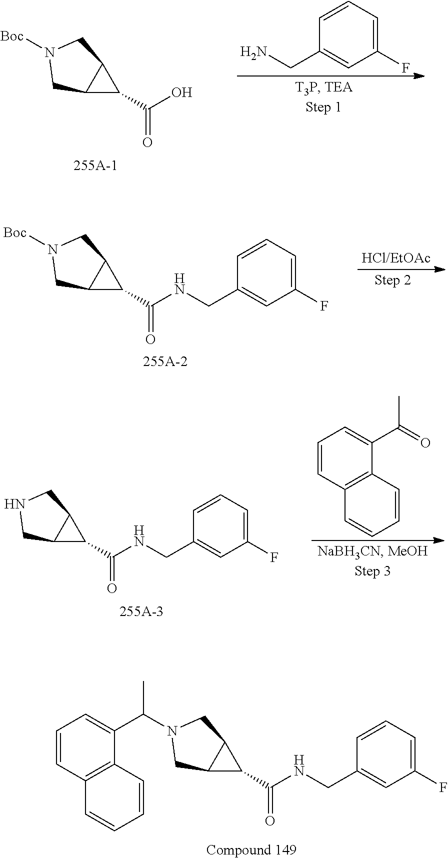 [SMILES: CC(C)(C)OC(=O)N1C[C@@H]2[C@H](C1)[C@H]2C(=O)O ; NCc1cccc(F)c1 ; CC(C)(C)OC(=O)N1C[C@@H]2[C@H](C1)[C@H]2C(=O)NCc1cccc(F)c1 ; O=C(NCc1cccc(F)c1)[C@H]1[C@@H]2CNC[C@@H]21 ; CC(=O)c1cccc2ccccc12 ; CC(c1cccc2ccccc12)N1C[C@@H]2[C@H](C1)[C@H]2C(=O)NCc1cccc(F)c1]

### Step 1: (1R,5S,6r)-tert-Butyl 6-((3-fluorobenzyl)carbamoyl)-3-azabicyclo[3.1.0]hexane-3-carboxylate (255A-2)

To a solution of (1R,5S,6r)-3-(tert-butoxycarbonyl)-3-azabicyclo[3.1.0]hexane-6-carboxylic acid (500 mg, 2.20 mmol, 1.0 eq) and (3-fluorophenyl)methanamine (275 mg, 2.20 mmol, 1.0 eq) in DCM (10 mL) were added TEA (445 mg, 4.40 mmol, 612 μL, 2.0 eq) and T₃P (1.68 g, 2.64 mmol, 1.57 mL, 50% purity in EtOAc, 1.2 eq). The mixture was stirred at 20° C. for 16 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. (1R,5S,6r)-tert-Butyl 6-((3-fluorobenzyl)carbamoyl)-3-azabicyclo[3.1.0]hexane-3-carboxylate (400 mg, 1.20 mmol, 54% yield) was obtained as a white solid. M+H⁺=335.2 (LCMS); ¹H NMR (400 MHZ, DMSO-d₆) δ 8.56 (br t, J=5.9 Hz, 1H), 7.47-7.27 (m, 1H), 7.15-6.96 (m, 3H), 4.28 (br d, J=5.9 Hz, 2H), 3.48 (br dd, J=4.1, 10.8 Hz, 2H), 3.34-3.24 (m, 2H), 1.87 (br s, 2H), 1.43 (t, J=3.1 Hz, 1H), 1.37 (s, 9H).

### Step 2: (1R,5S,6r)-N-(3-Fluorobenzyl)-3-azabicyclo[3.1.0]hexane-6-carboxamide (255A-3)

To a stirred solution of (1R,5S,6r)-tert-butyl 6-((3-fluorobenzyl)carbamoyl)-3-azabicyclo[3.1.0]hexane-3-carboxylate (200 mg, 598 μmol, 1.0 eq) in EtOAc (1.0 mL) was added HCl/EtOAc (4 M, 5.0 mL). The mixture was stirred at 25° C. for 3 h. LCMS indicated that the starting material was completely consumed, and the desired mass was detected. The mixture was concentrated under vacuum at 30° C. to give the crude product (1R,5S,6r)-N-(3-fluorobenzyl)-3-azabicyclo[3.1.0]hexane-6-carboxamide (200 mg, HCl salt), which was used in the next step without any further purification. M+H⁺=235.2 (LCMS).

### Step 3: (1R,5S,6r)-N-(3-Fluorobenzyl)-3-(1-(naphthalen-1-yl)ethyl)-3-azabicyclo[3.1.0]hexane-6-carboxamide (Compound 149)

To a solution of (1R,5S,6r)-N-(3-fluorobenzyl)-3-azabicyclo[3.1.0]hexane-6-carboxamide (100 mg, 369 μmol, 1.0 eq, HCl salt) in MeOH (5.0 mL) was added TEA (10.0 μL), followed by the addition of 1-(naphthalen-1-yl)ethanone (62.9 mg, 369 μmol, 56.1 μL, 1.0 eq). The resulting mixture was adjusted to pH 6 with a small amount of AcOH. The mixture was stirred at 20° C. for 30 min, then NaBH₃CN (69.6 mg, 1.11 mmol, 3.0 eq) was added. The resulting mixture was stirred at 20° C. for another 16 h. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 60%-90% B over 8 min; mobile phase A: 10 mM aqueous NH₄HCO₃, mobile phase B: acetonitrile). (1R,5S,6r)-N-(3-Fluorobenzyl)-3-(1-(naphthalen-1-yl)ethyl)-3-azabicyclo[3.1.0]hexane-6-carboxamide (27.9 mg, 71.8 μmol, 19% yield) was obtained as a white solid. M+H⁺=389.3 (LCMS); ¹H NMR (400 MHZ, CD₃OD) δ 8.45 (br d, J=8.0 Hz, 1H), 7.87-7.82 (m, 1H), 7.73 (d, J=8.1 Hz, 1H), 7.54 (d, J=6.9 Hz, 1H), 7.50-7.36 (m, 3H), 7.31 (dt, J=6.0, 7.9 Hz, 1H), 7.08 (d, J=7.8 Hz, 1H), 7.04-6.92 (m, 2H), 4.35 (s, 2H), 4.07 (q, J=6.4 Hz, 1H), 3.39 (d, J=9.0 Hz, 1H), 2.77 (d, J=9.4 Hz, 1H), 2.63 (dd, J=3.4, 8.9 Hz, 1H), 2.28 (dd, J=3.5, 9.4 Hz, 1H), 2.13 (t, J=2.8 Hz, 1H), 2.01-1.95 (m, 1H), 1.84-1.79 (m, 1H), 1.45 (d, J=6.6 Hz, 3H).

### Example 256: 2-(1-(Naphthalen-1-yl)ethyl)-1,2,3,4-tetrahydroisoquinolin-7-amine (Compound 135)

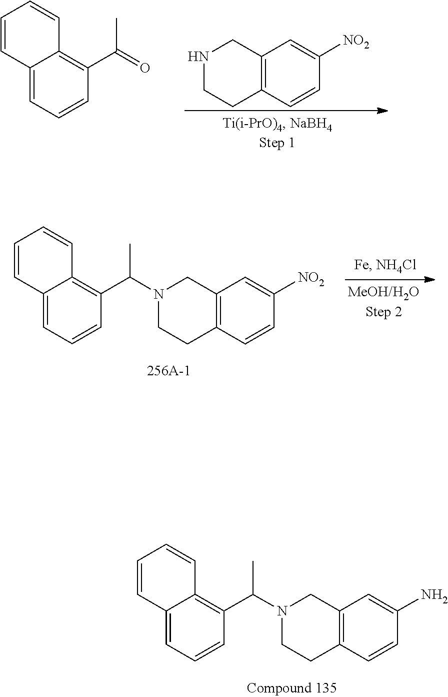 [SMILES: CC(=O)c1cccc2ccccc12 ; O=[N+]([O-])c1ccc2c(c1)CNCC2 ; CC(c1cccc2ccccc12)N1CCc2ccc([N+](=O)[O-])cc2C1 ; CC(c1cccc2ccccc12)N1CCc2ccc(N)cc2C1]

### Step 1: 2-(1-(Naphthalen-1-yl)ethyl)-7-nitro-1,2,3,4-tetrahydroisoquinoline (256A-1)

To a mixture of 1-(naphthalen-1-yl)ethanone (200 mg, 1.18 mmol, 178 μL, 1.0 eq) and 7-nitro-1,2,3,4-tetrahydroisoquinoline (230 mg, 1.29 mmol, 1.1 eq) in MeOH (5.0 mL) was added Ti(i-PrO)₄ (668 mg, 2.35 mmol, 694 μL, 2.0 eq). The mixture was stirred at 60° C. for 16 h. The mixture was cooled to 0° C. and NaBH₄ (δ8.9 mg, 2.35 mmol, 2.0 eq) was added. The resulting mixture was stirred at 20° C. for 2 h. LCMS indicated that the starting material was completely consumed, and the desired mass was detected. The reaction mixture was poured into ice water (6.0 mL) and extracted with EtOAc (3.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 75%-98% B over 8 min; mobile phase A: 10 mM aqueous NH₄HCO₃, mobile phase B: acetonitrile). 2-(1-(Naphthalen-1-yl)ethyl)-7-nitro-1,2,3,4-tetrahydroisoquinoline (100 mg, 301 μmol, 26% yield) was obtained as a white solid. M+H=333.2 (LCMS); ¹H NMR (400 MHZ, CDCl₃) δ 8.42 (br s, 1H), 8.02-7.94 (m, 1H), 7.93-7.86 (m, 2H), 7.80 (br d, J=8.1 Hz, 1H), 7.66 (br d, J=6.4 Hz, 1H), 7.53-7.42 (m, 3H), 7.23 (d, J=8.4 Hz, 1H), 4.36 (br s, 1H), 4.01 (br d, J=14.6 Hz, 1H), 3.73 (br d, J=14.9 Hz, 1H), 3.05-2.64 (m, 4H), 1.62 (br d, J=6.5 Hz, 3H).

### Step 2: 2-(1-(Naphthalen-1-yl)ethyl)-1,2,3,4-tetrahydroisoquinolin-7-amine (Compound 135)

To a solution of 2-(1-(naphthalen-1-yl)ethyl)-7-nitro-1,2,3,4-tetrahydroisoquinoline (70.0 mg, 211 μmol, 1.0 eq) in a mixture of MeOH (2.5 mL) and H₂O (0.5 mL) were added iron powder (58.8 mg, 1.05 mmol, 5.0 eq) and NH₄Cl (56.3 mg, 1.05 mmol, 5.0 eq). The mixture was stirred at 80° C. for 2 h. LCMS indicated that the starting material was completely consumed, and the desired mass was detected. The reaction mixture was allowed to cool to room temperature, then poured into H₂O (6.0 mL). The product was extracted with EtOAc (3.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 60%-95% B over 8 min; mobile phase A: 10 mM aqueous NH₄HCO₃, mobile phase B: acetonitrile). 2-(1-(Naphthalen-1-yl)ethyl)-1,2,3,4-tetrahydroisoquinolin-7-amine (3.40 mg, 10.3 μmol, 5% yield) was obtained as a white gum. M+H⁺=303.1 (LCMS); ¹H NMR (400 MHz, CDCl₃) δ 8.48 (br s, 1H), 7.90-7.83 (m, 1H), 7.77 (d, J=7.9 Hz, 1H), 7.69 (br d, J=7.3 Hz, 1H), 7.52-7.42 (m, 3H), 6.89 (d, J=8.2 Hz, 1H), 6.51 (br d, J=8.0 Hz, 1H), 6.36 (s, 1H), 4.25 (br d, J=6.0 Hz, 1H), 3.86 (br d, J=14.7 Hz, 1H), 3.56 (br d, J=15.1 Hz, 2H), 2.86-2.57 (m, 4H), 1.58 (br s, 3H).

### Example 257: N-(2-(1-(Naphthalen-1-yl)ethyl)-1,2,3,4-tetrahydroisoquinolin-7-yl)-2-phenylacetamide (Compound 139)

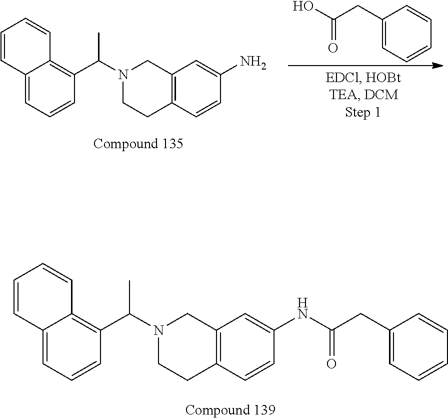 [SMILES: CC(c1cccc2ccccc12)N1CCc2ccc(N)cc2C1 ; O=C(O)Cc1ccccc1 ; CC(c1cccc2ccccc12)N1CCc2ccc(NC(=O)Cc3ccccc3)cc2C1]

### Step 1: N-(2-(1-(Naphthalen-1-yl)ethyl)-1,2,3,4-tetrahydroisoquinolin-7-yl)-2-phenylacetamide (Compound 139)

To a solution of 2-(1-(naphthalen-1-yl)ethyl)-1,2,3,4-tetrahydroisoquinolin-7-amine (40.0 mg, 132 μmol, 1.0 eq) and 2-phenylacetic acid (21.7 mg, 159 μmol, 20.0 μL, 1.2 eq) in DCM (2.0 mL) were added TEA (26.8 mg, 265 μmol, 368 μL, 2.0 eq), EDCI (30.4 mg, 159 μmol, 1.2 eq) and HOBt (21.5 mg, 159 μmol, 1.2 eq). The mixture was stirred at 20° C. for 3 h. LCMS indicated that the starting material was completely consumed, and the desired mass was detected. The reaction mixture was poured into H₂O (5.0 mL) and extracted with EtOAc (5.0 mL×3). The combined organic layers were dried over Na₂SO₄, filtered, and concentrated under vacuum to give a residue which was purified by preparative HPLC (Phenomenex Luna C18 column (150×30 mm, 5 μm); flow rate: 25 mL/min; gradient: 27%-57% B over 8 min; mobile phase A: 0.1% aqueous TFA, mobile phase B: acetonitrile). N-(2-(1-(Naphthalen-1-yl)ethyl)-1,2,3,4-tetrahydroisoquinolin-7-yl)-2-phenylacetamide (7.04 mg, 12.9 μmol, 10% yield, TFA salt) was obtained as a yellow solid. M+H⁺=421.1 (LCMS); ¹H NMR (400 MHZ, CD₃OD) δ8.16-7.97 (m, 2H), 7.85 (br s, 1H), 7.66 (br t, J=7.8 Hz, 4H), 7.32 (br s, 8H), 5.58 (br d, J=6.6 Hz, 1H), 4.77-3.94 (m, 2H), 3.86-3.38 (m, 4H), 3.29-2.66 (m, 2H), 1.95 (d, J=6.8 Hz, 3H).

### Example 258: 5-((1,2-Dimethylazetidin-2-yl)methoxy)-2-methyl-N-(1-(naphthalen-1-yl)cyclopropyl)benzamide (Compound 404)

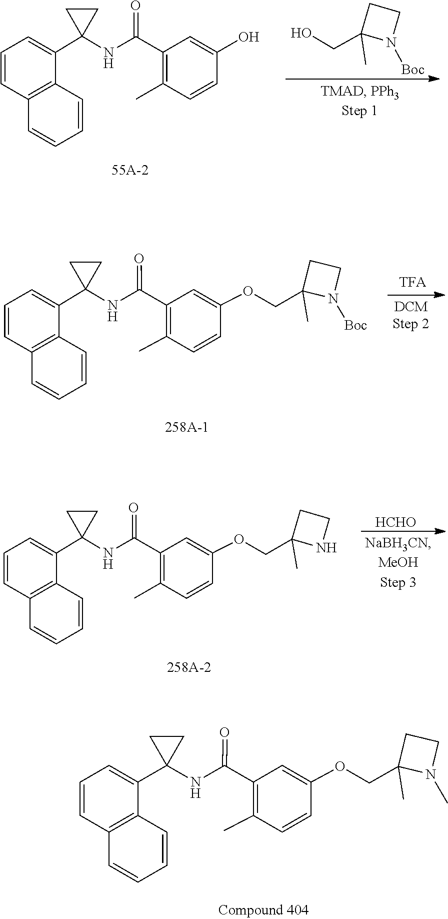

### Step 1: tert-Butyl 2-methyl-2-((4-methyl-3-((1-(naphthalen-1-yl)cyclopropyl)carbamoyl)phenoxy)methyl)azetidine-1-carboxylate (258A-1)

To a solution 5-hydroxy-2-methyl-N-(1-(naphthalen-1-yl)cyclopropyl)benzamide (300 mg, 945 μmol, 1.0 eq) and tert-butyl 2-(hydroxymethyl)-2-methylazetidine-1-carboxylate (190 mg, 945 μmol, 1.0 eq) in toluene (18 mL) were added TMAD (488 mg, 2.84 mmol, 3.0 eq) and PPh₃ (744 mg, 2.84 mmol, 3.0 eq). The mixture was degassed and purged with N₂ three times, then stirred at 100° C. for 16 h under a N₂ atmosphere. LCMS indicated that the starting material was completely consumed, and the desired mass was detected. The reaction mixture was allowed to cool to room temperature, poured into H₂O (15 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. tert-Butyl 2-methyl-2-((4-methyl-3-((1-(naphthalen-1-yl)cyclopropyl)carbamoyl)phenoxy)methyl)azetidine-1-carboxylate (370 mg, 740 μmol, 79% yield) was obtained as a yellow solid. M+H⁺=501.3 (LCMS).

### Step 2: 2-Methyl-5-((2-methylazetidin-2-yl)methoxy)-N-(1-(naphthalen-1-yl)cyclopropyl)benzamide (258A-2)

To a solution of tert-butyl 2-methyl-2-((4-methyl-3-((1-(naphthalen-1-yl)cyclopropyl) carbamoyl)phenoxy)methyl)azetidine-1-carboxylate (180 mg, 360 μmol, 1.0 eq) in DCM (10 mL) was added TFA (3.6 mL). The mixture was stirred at 20° C. for 1 h. LCMS indicated that the starting material was completely consumed, and the desired mass was detected. The reaction mixture was concentrated under vacuum at 30° C. to give the product 2-methyl-5-((2-methyl azetidin-2-yl)methoxy)-N-(1-(naphthalen-1-yl)cyclopropyl)benzamide (90.0 mg, TFA salt) as a brown oil, which was used in the next step without any further purification. M+H⁺=401.1 (LCMS).

### Step 3: 5-((1,2-Dimethylazetidin-2-yl)methoxy)-2-methyl-N-(1-(naphthalen-1-yl)cyclopropyl)benzamide (Compound 404)

To a solution of 2-methyl-5-((2-methylazetidin-2-yl)methoxy)-N-(1-(naphthalen-1-yl)cyclopropyl)benzamide (80.0 mg, 155 μmol, 1.0 eq, TFA salt) in MeOH (5.0 mL) was added TEA (10.0 μL), followed by the addition of formaldehyde (9.34 mg, 311 μmol, 8.57 μL, 2.0 eq). The resulting mixture was treated with a small amount of AcOH to adjust the pH to 6, then NaBH₃CN (19.5 mg, 311 μmol, 2.0 eq) was added. The mixture was stirred at 20° C. for 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 10%-50% B over 8 min; mobile phase A: 0.04% aqueous HCl, mobile phase B: acetonitrile). 2-Methyl-5-((2-methylazetidin-2-yl)methoxy)-N-(1-(naphthalen-1-yl)cyclopropyl)benzamide (33.0 mg, 73.2 μmol, 47% yield, HCl salt) was obtained as a yellow solid. M+H⁺=415.2 (LCMS); ¹H NMR (400 MHZ, DMSO-d₆) δ 11.32-10.41 (m, 1H), 9.13 (s, 1H), 8.67 (d, J=8.4 Hz, 1H), 7.93 (d, J=7.6 Hz, 1H), 7.87-7.78 (m, 2H), 7.64-7.41 (m, 3H), 7.13-7.04 (m, 1H), 6.99-6.88 (m, 1H), 6.80-6.68 (m, 1H), 4.52-4.29 (m, 1H), 4.25-3.98 (m, 1H), 4.00-3.73 (m, 2H), 2.67-2.56 (m, 3H), 2.45-2.03 (m, 2H), 1.95 (s, 3H), 1.59 (s, 3H), 1.37 (br s, 2H), 1.18 (br s, 2H).

### Example 259: rac-5-((1S,2R)-2-Aminocyclobutoxy)-2-methyl-N-(1-(naphthalen-1-yl)cyclopropyl)benzamide (Compound 406)

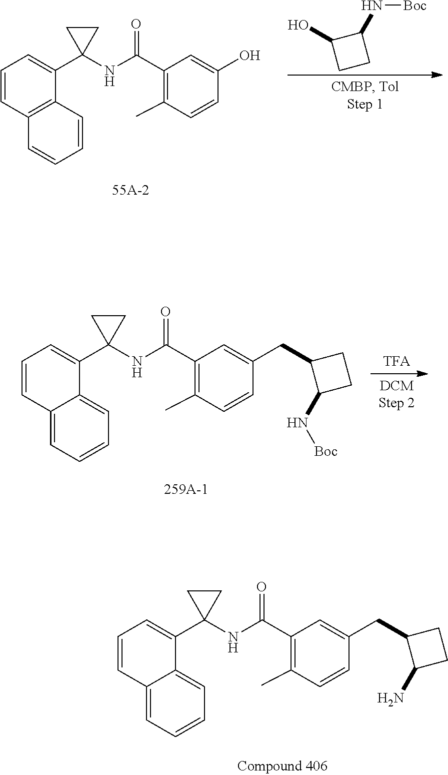

### Step 1: rac-tert-Butyl((1R,2S)-2-(4-methyl-3-((1-(naphthalen-1-yl)cyclopropyl)carbamoyl)phenoxy)cyclobutyl)carbamate (259A-1)

To a solution of rac-tert-butyl((1S,2R)-2-hydroxycyclobutyl)carbamate (118 mg, 630 μmol, 1.0 eq) and 5-hydroxy-2-methyl-N-(1-(naphthalen-1-yl)cyclopropyl)benzamide (200 mg, 630 μmol, 1.0 eq) in toluene (12 mL) was added CMBP (228 mg, 945 μmol, 1.5 eq). The mixture was degassed and purged with N₂ three times and stirred at 100° C. for 16 h under a N₂ atmosphere. LCMS indicated that the starting material was completely consumed, and the desired mass was detected. The reaction mixture was allowed to cool to room temperature, poured into H₂O (8.0 mL) and extracted with EtOAc (5.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 45%-85% B over 8 min; mobile phase A: 0.04% aqueous HCl, mobile phase B: acetonitrile). rac-tert-Butyl((1R,2S)-2-(4-methyl-3-((1-(naphthalen-1-yl)cyclopropyl)carbamoyl)phenoxy)cyclobutyl)carbamate (46.0 mg, 94.53 μmol, 5% yield) was obtained as a red solid. M+H⁺=487.3 (LCMS).

### Step 2: rac-5-((1S,2R)-2-Aminocyclobutoxy)-2-methyl-N-(1-(naphthalen-1-yl)cyclopropyl)benzamide (Compound 406)

To a solution of rac-tert-butyl((1R,2S)-2-(4-methyl-3-((1-(naphthalen-1-yl)cyclopropyl) carbamoyl)phenoxy)cyclobutyl)carbamate (46.0 mg, 94.5 μmol, 1.0 eq) in DCM (5.0 mL) was added TFA (1.0 mL). The mixture was stirred at 20° C. for 1 h. LCMS indicated that the starting material was completely consumed, and the desired mass was detected. The reaction mixture was concentrated under vacuum at 30° C. to give a residue which was purified by preparative HPLC (Phenomenex Luna C18 column (80×30 mm, 3 μm); flow rate: 25 mL/min; gradient: 1%-50% B over 8 min; mobile phase A: 0.04% aqueous HCl, mobile phase B: acetonitrile). rac-5-((1S,2R)-2-Aminocyclobutoxy)-2-methyl-N-(1-(naphthalen-1-yl)cyclopropyl)benzamide (10.1 mg, 23.7 μmol, 25% yield, HCl salt) was obtained as a white solid. M+H⁺=387.1 (LCMS); ¹H NMR (400 MHZ, DMSO-d₆) δ 9.18-9.08 (m, 1H), 8.71-8.61 (m, 1H), 8.30-8.15 (m, 3H), 7.98-7.91 (m, 1H), 7.87-7.78 (m, 2H), 7.62-7.42 (m, 3H), 7.10-7.03 (m, 1H), 6.84-6.77 (m, 1H), 6.60-6.54 (m, 1H), 4.86-4.74 (m, 1H), 4.03-3.90 (m, 1H), 2.38-2.23 (m, 1H), 2.20-2.07 (m, 2H), 2.02-1.95 (m, 3H), 1.94-1.87 (m, 1H), 1.40-1.32 (m, 2H), 1.23-1.14 (m, 2H).

### Example 260: rac-5-(((2R,4R)-4-Hydroxypyrrolidin-2-yl)methoxy)-2-methyl-N-(1-(naphthalen-1-yl)cyclopropyl)benzamide (Compound 416)

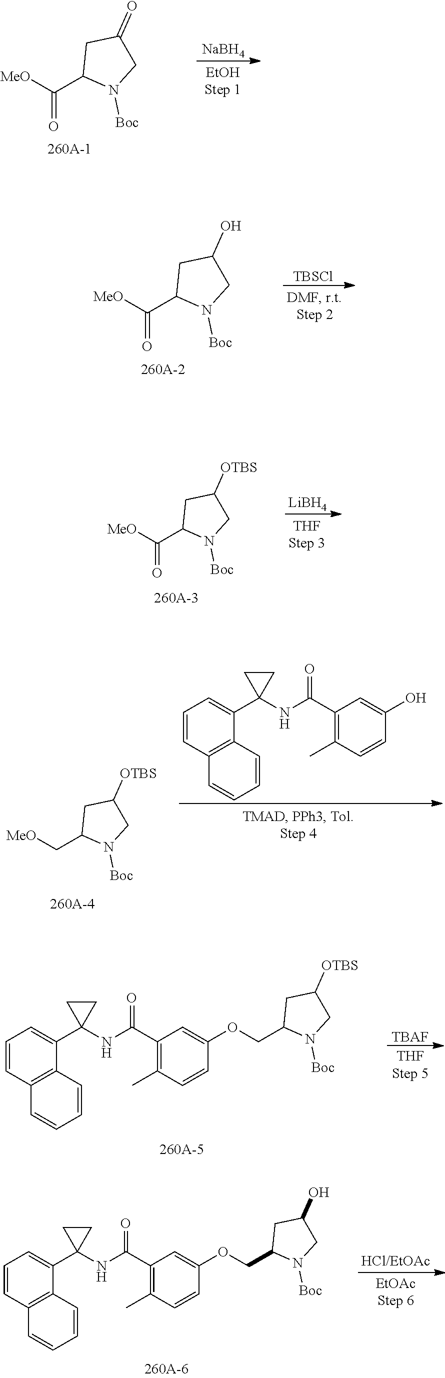

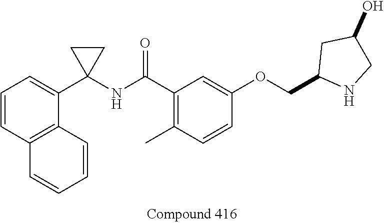

### Step 1: 1-tert-Butyl 2-methyl 4-hydroxypyrrolidine-1,2-dicarboxylate (260A-2)

To a solution of 1-ter-butyl 2-methyl 4-oxopyrrolidine-1,2-dicarboxylate (5.00 g, 20.6 mmol, 1.0 eq) in EtOH (50.0 mL) was added sodium tetrahydroborate (778 mg, 20.6 mmol, 1.0 eq) in portions at 0° C. The mixture was stirred at 0° C. for 30 min under a N₂ atmosphere. LCMS indicated that the starting material was completely consumed, and the desired mass was detected. The reaction mixture was poured into cold H₂O (10 mL) and extracted with EtOAc (10 mL×3). The combined organic layers were dried over Na₂SO₄, filtered, and concentrated under vacuum to give a residue which was purified by column chromatography using a gradient of EtOAc/petroleum ether from 1/10 to 1/1. 1-tert-Butyl 2-methyl 4-hydroxypyrrolidine-1,2-dicarboxylate (1.30 g, 5.30 mmol, 26% yield) was obtained as a white solid. M−56+H⁺=190.2 (LCMS); ¹H NMR (400 MHZ, CDCl₃) δ 4.38-4.35 (m, 1H), 3.79 (s, 3H), 3.75-3.57 (m, 3H), 3.52-3.46 (m, 1H), 2.11 (br d, J=7.8 Hz, 1H), 1.43 (s, 9H).

### Step 2: 1-tert-Butyl2-methyl 4-((tert-butyldimethylsilyl)oxy)pyrrolidine-1,2-dicarboxylate (260A-3)

To a stirred solution of 1-tert-butyl 2-methyl 4-hydroxypyrrolidine-1,2-dicarboxylate (300 mg, 1.22 mmol, 1.0 eq) in DMF (3.0 mL) were added imidazole (167 mg, 2.45 mmol, 2.0 eq) and TBSCl (221 mg, 1.47 mmol, 1.2 eq) at 0° C. in portions. The mixture was stirred at 20° C. for 16 h. LCMS indicated that the starting material was completely consumed, and the desired mass was detected. The mixture was poured into H₂O (10 mL) and extracted with EtOAc (10 mL×3). The combined organic layers were dried over Na₂SO₄, filtered, and concentrated under vacuum to give a residue which was purified by column chromatography using a gradient of DCM. 1-tert-Butyl 2-methyl 4-((tert-butyldimethylsilyl)oxy)pyrrolidine-1,2-dicarboxylate (400 mg, 1.11 mmol, 91% yield, HCl salt) was obtained as a white solid. M+H⁺=360.1 (LCMS); ¹H NMR (400 MHZ, CDCl₃) δ 4.40-4.25 (m, 2H), 3.71 (s, 3H), 3.67-3.56 (m, 1H), 3.39-3.24 (m, 1H), 2.37-2.22 (m, 1H), 2.15-2.06 (m, 1H), 1.43 (s, 9H), 0.92-0.86 (m, 9H), 0.07-0.03 (m, 6H).

### Step 3: tert-Butyl 4-((tert-butyldimethylsilyl)oxy)-2-(methoxymethyl)pyrrolidine-1-carboxylate (260A-4)

To a solution of 1-tert-Butyl 2-methyl 4-((tert-butyldimethylsilyl)oxy)pyrrolidine-1,2-dicarboxylate (400 mg, 1.11 mmol, 1.0 eq) in THF (5.0 mL) was added lithium borohydride (60.6 mg, 2.78 mmol, 2.5 eq) in portions at 0° C. The mixture was stirred at 25° C. for 16 h under a N₂ atmosphere. LCMS indicated that the starting material was completely consumed, and the desired mass was detected. The reaction mixture was poured into saturated aqueous NH₄Cl (10 mL) and extracted with EtOAc (10 mL×3). The combined organic layers were dried over Na₂SO₄, filtered, and concentrated under vacuum to give a residue which was purified by column chromatography using a gradient of DCM to give tert-butyl 4-((tert-butyldimethylsilyl)oxy)-2-(methoxymethyl)pyrrolidine-1-carboxylate (200 mg, 603 μmol, 54% yield) as a yellow oil. M+H⁺=332.2 (LCMS); ¹H NMR (400 MHZ, CDCl₃) δ 4.45-4.25 (m, 1H), 4.15-3.96 (m, 1H), 3.88-3.76 (m, 1H), 3.74-3.65 (m, 1H), 3.55 (br dd, J=7.4, 11.3 Hz, 1H), 3.36-3.20 (m, 1H), 3.17-3.04 (m, 1H), 2.34-2.14 (m, 1H), 1.65-1.56 (m, 1H), 1.48 (s, 9H), 0.90 (s, 9H), 0.09 (br s, 6H).

### Step 4: tert-Butyl 4-((tert-butyldimethylsilyl)oxy)-2-((4-methyl-3-((1-(naphthalen-1-yl)cyclopropyl)carbamoyl)phenoxy)methyl)pyrrolidine-1-carboxylate (260A-5)

A mixture of tert-butyl 4-((tert-butyldimethylsilyl)oxy)-2-(methoxymethyl)pyrrolidine-1-carboxylate (190 mg, 573 μmol, 1.0 eq), 5-hydroxy-2-methyl-N-(1-(naphthalen-1-yl)cyclopropyl)benzamide (182 mg, 573 μmol, 1.0 eq) and PPh₃ (4.34 g, 16.6 mmol, 1.1 eq) in toluene (2.0 mL) was degassed and purged with N₂ three times. To the mixture was added TMAD (296 mg, 1.72 mmol, 3.0 eq) in portions at 20° C. The resulting mixture was stirred at 100° C. for 16 h under a N₂ atmosphere. LCMS indicated that the starting material was completely consumed, and the desired mass was detected. The reaction mixture was allowed to cool to room temperature, poured into H₂O (30 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/9. tert-Butyl 4-((tert-butyldimethylsilyl)oxy)-2-((4-methyl-3-((1-(naphthalen-1-yl)cyclopropyl)carbamoyl)phenoxy)methyl)pyrrolidine-1-carboxylate (150 mg, 238 μmol, 41% yield) was obtained as a yellow oil. M+H⁺=631.4 (LCMS); ¹H NMR (400 MHZ, DMSO-d₆) δ 9.10-9.02 (m, 1H), 8.65 (br d, J=8.3 Hz, 1H), 7.92 (d, J=8.0 Hz, 1H), 7.88-7.75 (m, 2H), 7.59-7.39 (m, 3H), 7.02 (d, J=8.3 Hz, 1H), 6.83 (br dd, J=2.3, 8.3 Hz, 1H), 6.61 (br d, J=12.0 Hz, 1H), 4.48-4.33 (m, 1H), 4.13-4.05 (m, 1H), 3.97-3.91 (m, 1H), 3.50 (dd, J=5.1, 11.6 Hz, 1H), 3.31-3.22 (m, 1H), 3.15-3.00 (m, 1H), 2.15-2.02 (m, 1H), 1.95 (s, 3H), 1.91-1.80 (m, 1H), 1.45-1.29 (m, 11H), 1.17-1.13 (m, 2H), 0.74 (br d, J=8.0 Hz, 9H), 0.06-0.08 (m, 6H).

### Step 5: rac-(2R,4R)-tert-Butyl-4-hydroxy-2-((4-methyl-3-((1-(naphthalen-1-yl)cyclo propyl)carbamoyl)phenoxy)methyl)pyrrolidine-1-carboxylate (260A-6)

A solution of tert-butyl 4-((tert-butyldimethylsilyl)oxy)-2-((4-methyl-3-((1-(naphthalen-1-yl)cyclopropyl)carbamoyl)phenoxy)methyl)pyrrolidine-1-carboxylate (150 mg, 238 μmol, 1.0 eq) in THF (2.0 mL) was degassed and purged with N₂ three times. To the mixture was added tetrabutylammonium fluoride (1 M in THF, 713 μmol, 3.0 eq) dropwise at 0° C. The resulting mixture was stirred at 20° C. for 3 h under a N₂ atmosphere. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by preparative TLC (EtOAc/petroleum ether=1/2, R_{f}=0.6). rac-(2R,4R)-tert-Butyl 4-hydroxy-2-((4-methyl-3-((1-(naphthalen-1-yl)cyclopropyl)carbamoyl)phenoxy)methyl)pyrrolidine-1-carboxylate (80.0 mg, 155 μmol, 65% yield) was obtained as a white solid. M+H⁺=517.3 (LCMS); ¹H NMR (400 MHZ, DMSO-d₆) δ 9.08 (s, 1H), 8.65 (br d, J=8.5 Hz, 1H), 7.92 (d, J=7.5 Hz, 1H), 7.82 (t, J=7.8 Hz, 2H), 7.60-7.42 (m, 3H), 7.02 (d, J=8.5 Hz, 1H), 6.89-6.81 (m, 1H), 6.62 (br d, J=14.5 Hz, 1H), 5.01 (br s, 1H), 4.24 (br s, 1H), 4.10 (br s, 1H), 3.96 (br d, J=4.9 Hz, 2H), 3.44 (dd, J=4.8, 11.4 Hz, 1H), 3.19-3.07 (m, 1H), 2.09-2.01 (m, 1H), 1.94 (s, 3H), 1.87 (br d, J=13.5 Hz, 1H), 1.48-1.30 (m, 9H), 1.30 (br s, 2H), 1.17-1.15 (m, 2H).

### Step 6: rac-5-(((2R,4R)-4-Hydroxypyrrolidin-2-yl)methoxy)-2-methyl-N-(1-(naphthalen-1-yl)cyclopropyl)benzamide (Compound 416)

To a stirred solution of rac-(2R,4R)-tert-butyl 4-hydroxy-2-((4-methyl-3-((1-(naphthalen-1-yl)cyclopropyl)carbamoyl)phenoxy)methyl)pyrrolidine-1-carboxylate (80.0 mg, 155 μmol, 1.0 eq) in EtOAc (1.0 mL) was added HCl/EtOAc (4 M, 3.0 mL). The mixture was stirred at 20° C. for 3 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 5%-40% B over 8 min; mobile phase A: 0.04% aqueous HCl, mobile phase B: acetonitrile). rac-5-(((2R,4R)-4-Hydroxypyrrolidin-2-yl)methoxy)-2-methyl-N-(1-(naphthalen-1-yl)cyclopropyl)benzamide (140 mg, 334 μmol, 77% yield, HCl salt) was obtained as a white solid. M+H⁺=417.1 (LCMS); ¹H NMR (400 MHZ, DMSO-d₆) δ9.61-9.45 (m, 1H), 9.12 (s, 1H), 9.10-8.97 (m, 1H), 8.66 (d, J=8.4 Hz, 1H), 7.93 (d, J=8.0 Hz, 1H), 7.87-7.77 (m, 2H), 7.61-7.42 (m, 3H), 7.08 (d, J=8.4 Hz, 1H), 6.88 (dd, J=2.6, 8.4 Hz, 1H), 6.66 (d, J=2.5 Hz, 1H), 5.46 (br s, 1H), 4.39 (br s, 1H), 4.21-4.13 (m, 1H), 4.07 (t, J=9.9 Hz, 1H), 3.89 (br d, J=3.9 Hz, 1H), 3.22-3.11 (m, 1H), 3.10-3.01 (m, 1H), 2.32-2.22 (m, 1H), 1.97 (s, 3H), 1.72-1.59 (m, 1H), 1.36 (br s, 2H), 1.18 (br s, 2H).

### Example 261: 5-(((2R,4S)-4-Hydroxypyrrolidin-2-yl)methoxy)-2-methyl-N-(1-(naphthalen-1-yl)cyclopropyl)benzamide (Compound 414)

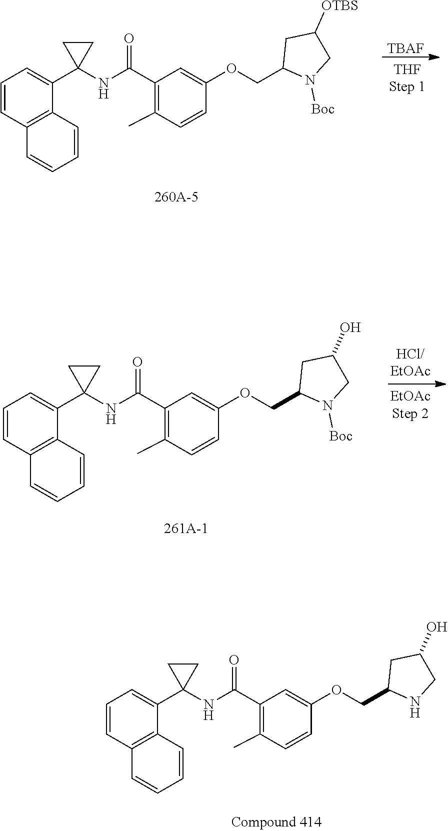

### Step 1: rac-(2R,4S)-tert-Butyl4-hydroxy-2-((4-methyl-3-((1-(naphthalen-1-yl)cyclo propyl)carbamoyl)phenoxy)methyl)pyrrolidine-1-carboxylate (261A-1)

A solution of tert-Butyl 4-((tert-butyldimethylsilyl)oxy)-2-((4-methyl-3-((1-(naphthalen-1-yl)cyclopropyl)carbamoyl)phenoxy)methyl)pyrrolidine-1-carboxylate (150 mg, 238 μmol, 1.0 eq) in THF (2.0 mL) was degassed and purged with N₂ three times. To the mixture was added tetrabutylammonium fluoride (1 M in THF, 713 μmol, 3.0 eq) dropwise at 0° C. The resulting mixture was stirred at 20° C. for 3 h under a N₂ atmosphere. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by preparative TLC (EtOAc/petroleum ether=1/2, R_{f}=0.6). rac-(2R,4S)-tert-Butyl 4-hydroxy-2-((4-methyl-3-((1-(naphthalen-1-yl)cyclopropyl)carbamoyl)phenoxy) methyl)pyrrolidine-1-carboxylate (40.0 mg, 77.4 μmol, 33% yield) was obtained as a white solid. M+H⁺=517.2 (LCMS); ¹H NMR (400 MHZ, DMSO-d₆) δ 9.07 (s, 1H), 8.65 (br d, J=8.1 Hz, 1H), 7.93 (d, J=7.9 Hz, 1H), 7.82 (t, J=7.6 Hz, 2H), 7.59-7.43 (m, 3H), 7.02 (br d, J=7.8 Hz, 1H), 6.83 (dd, J=2.4, 8.3 Hz, 1H), 6.61 (br s, 1H), 4.91 (br s, 1H), 4.25 (br s, 1H), 4.00 (br d, J=3.8 Hz, 1H), 3.96-3.81 (m, 1H), 3.31-3.19 (m, 2H), 1.94 (br s, 5H), 1.45-1.31 (m, 9H), 1.30 (br s, 2H), 1.16 (br s, 2H).

### Step 2: rac-5-(((2R,4S)-4-Hydroxypyrrolidin-2-yl)methoxy)-2-methyl-N-(1-(naphthalen-1-yl)cyclopropyl)benzamide (Compound 414)

To a stirred solution of rac-(2R,4S)-tert-Butyl 4-hydroxy-2-((4-methyl-3-((1-(naphthalen-1-yl)cyclopropyl)carbamoyl)phenoxy)methyl)pyrrolidine-1-carboxylate (40.0 mg, 77.4 μmol, 1.0 eq) in EtOAc (1.0 mL) was added HCl/EtOAc (4 M, 8.0 mL). The mixture was stirred at 20° C. for 3 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 5%-40% B over 8 min; mobile phase A: 0.04% aqueous HCl, mobile phase B: acetonitrile). rac-5-(((2R,4S)-4-Hydroxypyrrolidin-2-yl)methoxy)-2-methyl-N-(1-(naphthalen-1-yl)cyclopropyl)benzamide (5.70 mg, 13.5 μmol, 17% yield, HCl salt) was obtained as a white solid. M+H⁺=417.1 (LCMS); ¹H NMR (400 MHZ, DMSO-d₆) δ 9.55-9.36 (m, 1H), 9.10 (s, 1H), 8.91-8.74 (m, 1H), 8.65 (d, J=7.9 Hz, 1H), 7.94 (d, J=7.8 Hz, 1H), 7.87-7.79 (m, 2H), 7.60-7.45 (m, 3H), 7.08 (d, J=8.4 Hz, 1H), 6.88 (dd, J=2.7, 8.2 Hz, 1H), 6.66 (d, J=2.5 Hz, 1H), 5.45-5.43 (m, 1H), 4.42 (br s, 1H), 4.18 (br d, J=7.3 Hz, 1H), 4.06-3.94 (m, 2H), 3.26-3.20 (m, 1H), 3.12-2.97 (m, 1H), 2.13-2.01 (m, 1H), 1.97 (s, 3H), 1.86-1.76 (m, 1H), 1.39-1.31 (m, 2H), 1.19 (br s, 2H).

### Example 262: 5-((2-Aminoethyl)amino)-2-methyl-N-(1-(naphthalen-1-yl)cyclopropyl)benzamide (Compound 412)

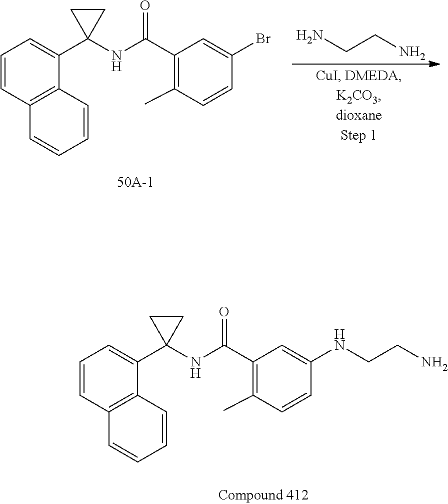

### Step 1: 5-((2-Aminoethyl)amino)-2-methyl-N-(1-(naphthalen-1-yl)cyclopropyl)benzamide (Compound 412)

To a stirred solution of 5-bromo-2-methyl-N-(1-(naphthalen-1-yl)cyclopropyl)benzamide (100 mg, 263 μmol, 1.0 eq) and ethane-1,2-diamine (23.7 mg, 394 μmol, 26.4 μL, 1.5 eq) in dioxane (1.0 mL) were added K₂CO₃ (36.3 mg, 263 μmol, 1.0 eq), CuI (5.01 mg, 26.3 μmol, 0.1 eq) and DMEDA (4.64 mg, 52.6 μmol, 5.66 μL, 0.2 eq) in one portion. The mixture was stirred at 100° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×3).

The mixture was concentrated under vacuum to give a residue which was purified by preparative HPLC (Phenomenex Luna column (80×30 mm, 3 μm); flow rate: 25 mL/min; gradient: 10%-50% B over 8 min; mobile phase A: 0.04% aqueous HCl, mobile phase B: acetonitrile). 5-((2-Aminoethyl)amino)-2-methyl-N-(1-(naphthalen-1-yl)cyclopropyl)benzamide (44.0 mg, 122 μmol, 47% yield, HCl salt) was obtained as a white solid. M+H⁺=360.1 (LCMS); ¹H NMR (400 MHZ, DMSO-d₆) δ 8.99 (s, 1H), 8.66 (d, J=8.4 Hz, 1H), 7.93 (d, J=7.6 Hz, 1H), 7.87-7.71 (m, 5H), 7.61-7.42 (m, 3H), 6.87 (d, J=8.4 Hz, 1H), 6.51 (dd, J=2.4, 8.2 Hz, 1H), 6.29 (d, J=2.4 Hz, 1H), 3.18 (t, J=6.3 Hz, 2H), 2.95-2.76 (m, 2H), 1.88 (s, 3H), 1.41-1.27 (m, 2H), 1.21-1.11 (m, 2H).

### Example 263: 5-((2-Aminoethyl)(methyl)amino)-2-methyl-N-(1-(naphthalen-1-yl)cyclopropyl)benzamide (Compound 421)

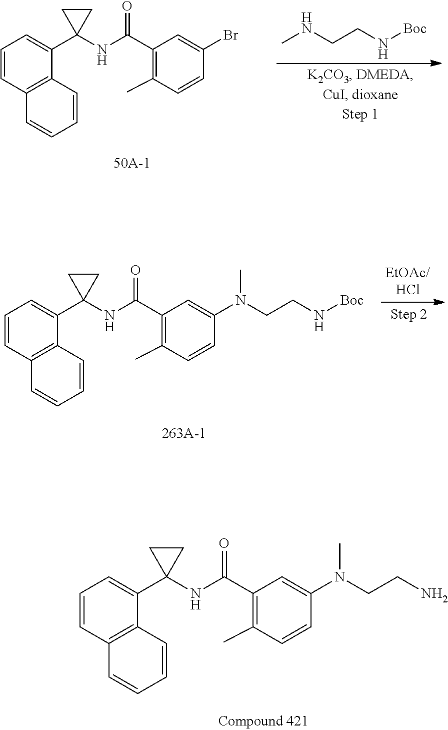

### Step 1: tert-Butyl(2-(methyl(4-methyl-3-((1-(naphthalen-1-yl)cyclopropyl)carbamoyl)phenyl)amino)ethyl)carbamate (263A-1)

A mixture of 5-bromo-2-methyl-N-(1-(naphthalen-1-yl)cyclopropyl)benzamide (150 mg, 394 μmol, 1.0 eq) and tert-butyl(2-(methylamino)ethyl)carbamate (103 mg, 592 μmol, 1.5 eq) in dioxane (7.5 mL) was degassed and purged with N₂ three times. To the mixture were added K₂CO₃ (54.5 mg, 394 μmol, 1.0 eq), DMEDA (6.96 mg, 78.9 μmol, 8.49 μL, 0.2 eq) and CuI (7.50 mg, 39.5 μmol, 0.1 eq) at 20° C. The resulting mixture was stirred at 100° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by was purified by preparative TLC (EtOAc/petroleum ether=1/1, R_{f}=0.3). tert-Butyl(2-(methyl(4-methyl-3-((1-(naphthalen-1-yl)cyclopropyl)carbamoyl)phenyl)amino)ethyl)carbamate (150 mg, 317 μmol, 80% yield) was obtained as a colorless oil. M+H⁺=474.2 (LCMS).

### Step 2: 5-((2-aminoethyl)(methyl)amino)-2-methyl-N-(1-(naphthalen-1-yl)cyclopropyl)benzamide (Compound 421)

To a stirred solution of tert-butyl(2-(methyl(4-methyl-3-((1-(naphthalen-1-yl)cyclopropyl) carbamoyl)phenyl)amino)ethyl)carbamate (100 mg, 211 μmol, 1.0 eq) in EtOAc (2.0 mL) was added HCl/EtOAc (4 M, 5.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 1%-35% B over 8 min; mobile phase A: 0.04% aqueous HCl, mobile phase B: acetonitrile). 5-((2-Aminoethyl)(methyl)amino)-2-methyl-N-(1-(naphthalen-1-yl)cyclopropyl)benzamide (25.8 mg, 60.2 μmol, 29% yield, HCl salt) was obtained as a white solid. M+H⁺=374.1 (LCMS); ¹H NMR (400 MHZ, DMSO-d₆) δ 8.99 (s, 1H), 8.60-8.71 (m, 3H), 7.93 (d, J=7.8 Hz, 1H), 7.86-7.78 (m, 2H), 7.60-7.43 (m, 3H), 6.87 (d, J=8.3 Hz, 1H), 6.54 (dd, J=2.5, 8.3 Hz, 1H), 6.32 (d, J=2.5 Hz, 1H), 3.27-3.19 (m, 2H), 3.02-2.91 (m, 2H), 2.53 (br d, J=3.9 Hz, 3H), 1.88 (s, 3H), 1.40-1.30 (m, 2H), 1.20-1.12 (m, 2H).

### Example 264: 2-Methyl-N-(1-(naphthalen-1-yl)cyclopropyl)-5-(pyrrolidin-3-yl)benzamide (Compound 413)

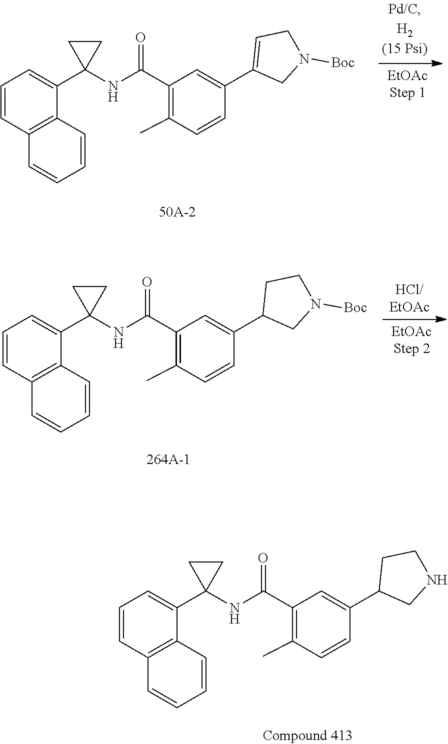

### Step 1: tert-Butyl 3-(4-methyl-3-((1-(naphthalen-1-yl)cyclopropyl)carbamoyl)phenyl)pyrrolidine-1-carboxylate (264A-1)

To a solution of tert-butyl 3-(4-methyl-3-((1-(naphthalen-1-yl)cyclopropyl)carbamoyl)phenyl)-2,5-dihydro-1H-pyrrole-1-carboxylate (150 mg, 288 μmol, 1.0 eq) in MeOH (2 mL) was added 10% palladium on carbon (10.0 mg). The mixture was stirred at 20° C. for 2 h under a H₂ (15 psi) atmosphere. LCMS indicated that the starting material was completely consumed, and the desired mass was detected. The reaction mixture was filtered through a pad of Celite and the filtrate was concentrated under vacuum to give the crude product tert-butyl 3-(4-methyl-3-((1-(naphthalen-1-yl)cyclopropyl)carbamoyl)phenyl)pyrrolidine-1-carboxylate (70.0 mg, 147 μmol, 51% yield) as a yellow oil, which was used in the next step without any further purification. M−56+H⁺=415.2 (LCMS).

### Step 2: 2-Methyl-N-(1-(naphthalen-1-yl)cyclopropyl)-5-(pyrrolidin-3-yl)benzamide (Compound 413)

To a stirred solution of tert-butyl 3-(4-methyl-3-((1-(naphthalen-1-yl)cyclopropyl)carbamoyl)phenyl)pyrrolidine-1-carboxylate (70.0 mg, 134 μmol, 1.0 eq) in EtOAc (1.0 mL) was added HCl/EtOAc (4 M, 5.0 mL). The mixture was stirred at 20° C. for 2 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 5%-40% B over 8 min; mobile phase A: 0.04% aqueous HCl, mobile phase B: acetonitrile). 2-Methyl-N-(1-(naphthalen-1-yl)cyclopropyl)-5-(pyrrolidin-3-yl)benzamide (20.2 mg, 54.0 μmol, 40% yield, HCl salt) was obtained as a white solid. M+H⁺=371.1 (LCMS); ¹H NMR (400 MHZ, DMSO-d₆) δ 9.11 (s, 1H), 9.03-8.86 (m, 1H), 8.65 (d, J=8.4 Hz, 1H), 7.94 (d, J=7.9 Hz, 1H), 7.83 (dd, J=4.6, 7.4 Hz, 2H), 7.62-7.43 (m, 3H), 7.22 (dd, J=1.5, 7.8 Hz, 1H), 7.12 (d, J=7.9 Hz, 1H), 7.02 (s, 1H), 3.56-3.47 (m, 1H), 3.37 (br s, 2H), 3.22-3.11 (m, 1H), 2.96 (br t, J=10.4 Hz, 1H), 2.30-2.20 (m, 1H), 1.99 (s, 3H), 1.90-1.77 (m, 1H), 1.36 (s, 2H), 1.24-1.14 (m, 2H).

### Example 265: 2-Methyl-N-(1-(naphthalen-1-yl)cyclopropyl)-5-(piperidin-4-yl)benzamide (Compound 407)

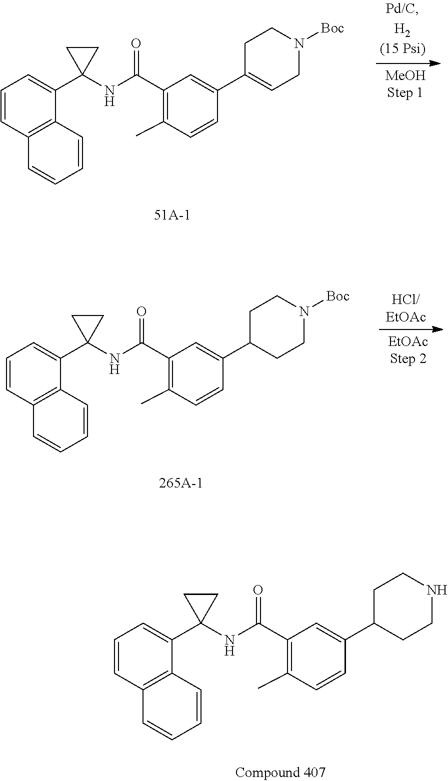

### Step 1: tert-Butyl 4-(4-methyl-3-((1-(naphthalen-1-yl)cyclopropyl)carbamoyl)phenyl)piperidine-1-carboxylate (265A-1)

To a solution of tert-butyl 4-(4-methyl-3-((1-(naphthalen-1-yl)cyclopropyl)carbamoyl)phenyl)-5,6-dihydropyridine-1 (2H)-carboxylate (150 mg, 280 μmol, 1.0 eq) in MeOH (2 mL) was added 10% palladium on carbon (10.0 mg). The mixture was stirred at 20° C. for 2 h under a H₂ (15 psi) atmosphere. LCMS indicated that the starting material was completely consumed, and the desired mass was detected. The reaction mixture was filtered through a pad of Celite and the filtrate was concentrated under vacuum to give a crude product tert-butyl 4-(4-methyl-3-((1-(naphthalen-1-yl)cyclopropyl)carbamoyl)phenyl) piperidine-1-carboxylate (70.0 mg, 142 μmol, 51% yield) as a yellow oil, which was used in the next step without any further purification. M+H⁺=485.3 (LCMS).

### Step 2: 2-Methyl-N-(1-(naphthalen-1-yl)cyclopropyl)-5-(piperidin-4-yl)benzamide (Compound 407)

To a stirred solution of tert-butyl 4-(4-methyl-3-((1-(naphthalen-1-yl)cyclopropyl)carbamoyl)phenyl) piperidine-1-carboxylate (70.0 mg, 130 μmol, 1.0 eq) in EtOAc (1.0 mL) was added HCl/EtOAc (4 M, 5.0 mL). The mixture was stirred at 20° C. for 2 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 5%-40% B over 8 min; mobile phase A: 0.04% aqueous HCl, mobile phase B: acetonitrile). 2-Methyl-N-(1-(naphthalen-1-yl)cyclopropyl)-5-(piperidin-4-yl)benzamide (47.7 mg, 122 μmol, 94% yield, HCl salt) was obtained as a white solid. M+H⁺=385.1 (LCMS); ¹H NMR (400 MHz, DMSO-d₆) δ 9.11 (s, 1H), 8.81-8.69 (m, 1H), 8.64 (d, J=8.4 Hz, 1H), 8.53-8.35 (m, 1H), 7.93 (d, J=8.0 Hz, 1H), 7.86-7.79 (m, 2H), 7.62-7.43 (m, 3H), 7.09 (s, 2H), 6.88 (s, 1H), 3.29 (br s, 2H), 2.99-2.85 (m, 2H), 2.79-2.69 (m, 1H), 1.98 (s, 3H), 1.88-1.78 (m, 2H), 1.76-1.63 (m, 2H), 1.40-1.32 (m, 2H), 1.23-1.13 (m, 2H).

### Example 266: 2-Methyl-N-(1-(naphthalen-1-yl)cyclopropyl)-5-(piperazin-1-yl)benzamide (Compound 410)

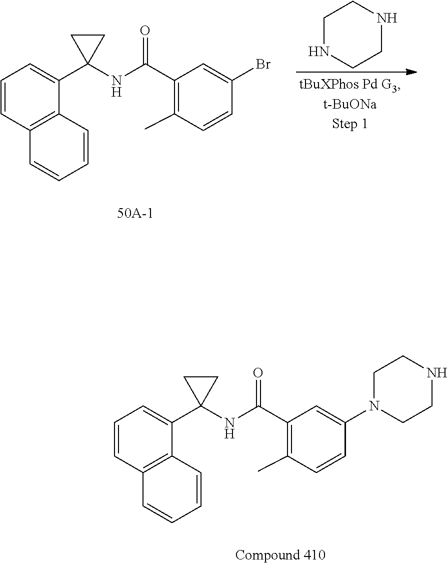

### Step 1: 2-Methyl-N-(1-(naphthalen-1-yl)cyclopropyl)-5-(piperazin-1-yl)benzamide (Compound 410)

To a solution of 5-bromo-2-methyl-N-(1-(naphthalen-1-yl)cyclopropyl)benzamide (200 mg, 526 μmol, 1.0 eq) and piperazine (68.0 mg, 789 μmol, 4.38 μL, 1.5 eq) in THF (10 mL) were added 1-BuONa (151.6 mg, 1.58 mmol, 3.0 eq) and/BuXPhos Pd G3 (41.8 mg, 52.6 μmol, 0.1 eq). The mixture was degassed and purged with N₂ three times and then the mixture was stirred at 80° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (4.0 mL×3). The combined organic layers were dried over Na₂SO₄, filtered, and concentrated under vacuum to give a residue which was purified by preparative HPLC (Phenomenex Waters Xbridge BEH C18 column (100×30 mm, 10 μm); flow rate: 25 mL/min; gradient: 25%-45% B over 8 min; mobile phase A: 0.05% aqueous NH₄HCO₃, mobile phase B: acetonitrile). 2-Methyl-N-(1-(naphthalen-1-yl)cyclopropyl)-5-(piperazin-1-yl)benzamide was obtained as a white solid. M+H⁺=386.1 (LCMS); ¹H NMR (400 MHZ, DMSO-d₆) δ 9.03 (s, 1H), 8.67 (d, J=7.8 Hz, 1H), 7.93 (d, J=7.8 Hz, 1H), 7.81 (dd, J=7.7, 12.3 Hz, 2H), 7.58-7.43 (m, 3H), 6.94 (d, J=8.6 Hz, 1H), 6.80 (dd, J=2.5, 8.4 Hz, 1H), 6.55 (d, J=2.6 Hz, 1H), 2.94-2.85 (m, 4H), 2.81-2.73 (m, 4H), 1.91 (s, 3H), 1.37-1.32 (m, 2H), 1.16 (br s, 2H).

### Example 267: 2-Methyl-5-(4-methylpiperazin-1-yl)-N-(1-(naphthalen-1-yl)cyclopropyl)benzamide (Compound 418)

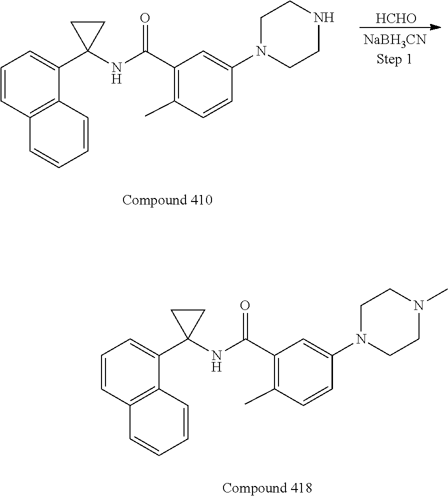

### Step 1: 2-Methyl-5-(4-methylpiperazin-1-yl)-N-(1-(naphthalen-1-yl)cyclopropyl)benzamide (Compound 418)

To a solution of 2-methyl-N-(1-(naphthalen-1-yl)cyclopropyl)-5-(piperazin-1-yl)benzamide (60.0 mg, 156 μmol, 1.0 eq) in MeOH (5.0 mL) was added TEA (10.0 μL), followed by the addition of formaldehyde (25.3 mg, 300 μmol, 23.2 μL, 37% purity in water, 2.0 eq). The resulting mixture was adjusted to pH 6 with a small amount of AcOH. The mixture was stirred at 20° C. for 30 min, then NaBH₃CN (19.6 mg, 311 μmol, 2.0 eq) was added. The reaction mixture was stirred at 20° C. for another 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 5%-35% B over 8 min; mobile phase A: 0.04% aqueous HCl, mobile phase B: acetonitrile). 2-Methyl-5-(4-methylpiperazin-1-yl)-N-(1-(naphthalen-1-yl)cyclopropyl)benzamide (17.9 mg, 40.59 μmol, 16% yield, HCl salt) was obtained as a white solid. M+H⁺=400.1 (LCMS); ¹H NMR (400 MHZ, DMSO-d₆) δ 10.21 (s, 1H), 9.06 (s, 1H), 8.68-8.62 (m, 1H), 7.93 (d, J=8.1 Hz, 1H), 7.82 (t, J=8.6 Hz, 2H), 7.58-7.44 (m, 3H), 7.01 (d, J=8.2 Hz, 1H), 6.90 (dd, J=2.5, 8.7 Hz, 1H), 6.65 (d, J=2.1 Hz, 1H), 3.68 (br d, J=11.9 Hz, 2H), 3.45 (br d, J=12.3 Hz, 2H), 3.13-3.02 (m, 2H), 2.90 (br t, J=12.7 Hz, 2H), 2.80 (d, J=4.8 Hz, 3H), 1.92 (s, 3H), 1.38-1.32 (m, 2H), 1.21-1.15 (m, 2H).

### Example 268: 5-(1,4-Diazepan-1-yl)-2-methyl-N-(1-(naphthalen-1-yl)cyclopropyl)benzamide (Compound 411)

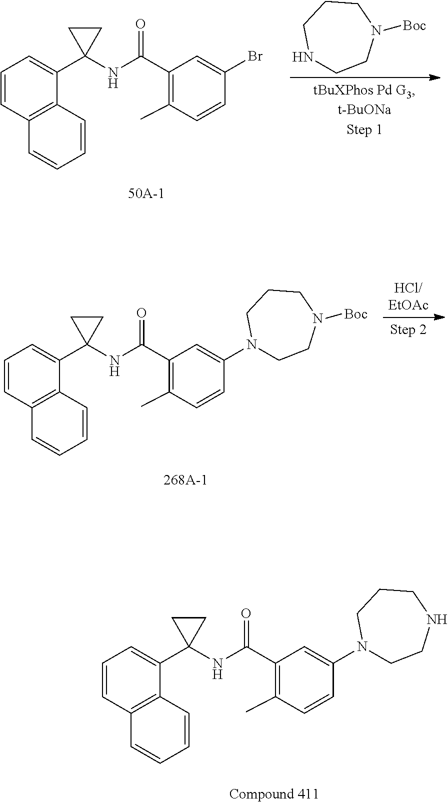

### Step 1: tert-Butyl 4-(4-methyl-3-((1-(naphthalen-1-yl)cyclopropyl)carbamoyl)phenyl)-1,4-diazepane-1-carboxylate (268A-1)

To a solution of 5-bromo-2-methyl-N-(1-(naphthalen-1-yl)cyclopropyl)benzamide (120 mg, 316 μmol, 1.0 eq) and tert-butyl 1,4-diazepane-1-carboxylate (94.8 mg, 473 μmol, 92.9 μL, 1.5 eq) in THF (10 mL) were added t-BuONa (91.0 mg, 947 μmol, 3.0 eq), tBuXPhos Pd G3 (25.1 mg, 31.6 μmol, 0.1 eq). The mixture was degassed and purged with N₂ three times and then the mixture was stirred at 80° C. for 3 h under a N₂ atmosphere. LCMS indicated that the starting material was completely consumed, and the desired mass was detected. The reaction mixture was allowed to cool to room temperature, poured into H₂O (10 mL) and extracted with EtOAc (4 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. tert-Butyl 4-(4-methyl-3-((1-(naphthalen-1-yl)cyclopropyl)carbamoyl)phenyl)-1,4-diazepane-1-carboxylate (80.0 mg, 160 μmol, 51% yield) was obtained as a yellow oil. M+H⁺=500.3 (LCMS); 1H NMR (400 MHZ, CDCl₃) δ 8.67 (d, J=8.6 Hz, 1H), 7.97 (d, J=6.4 Hz, 1H), 7.89 (br d, J=7.6 Hz, 2H), 7.81-7.75 (m, 2H), 6.90 (d, J=8.4 Hz, 2H), 6.60 (dd, J=2.8, 8.4 Hz, 1H), 6.33 (d, J=1.6 Hz, 1H), 3.74-3.66 (m, 2H), 3.50 (br t, J=5.8 Hz, 2H), 3.45-3.41 (m, 3H), 1.79-1.69 (m, 2H), 1.50 (br d, J=0.6 Hz, 2H), 1.41-1.34 (m, 6H), 1.22 (s, 9H).

### Step 2: 5-(1,4-Diazepan-1-yl)-2-methyl-N-(1-(naphthalen-1-yl)cyclopropyl)benzamide (Compound 411)

To a solution of tert-butyl 4-(4-methyl-3-((1-(naphthalen-1-yl)cyclopropyl)carbamoyl)phenyl)-1,4-diazepane-1-carboxylate (80.0 mg, 160 μmol, 51% yield) in EtOAc (2.0 mL) was added HCl/EtOAc (4 M, 2.0 mL). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm); flow rate: 25 mL/min; gradient: 5%-50% B over 8 min; mobile phase A: 0.04% aqueous HCl, mobile phase B: acetonitrile). 5-(1,4-Diazepan-1-yl)-2-methyl-N-(1-(naphthalen-1-yl)cyclopropyl)benzamide (13.9 mg, 31.9 μmol, 20% yield, HCl salt) was obtained as a white solid. M+H⁺=400.1 (LCMS); ¹H NMR (400 MHZ, DMSO-d₆) δ 9.01 (s, 3H), 8.67 (d, J=8.3 Hz, 1H), 7.93 (d, J=7.8 Hz, 1H), 7.86-7.75 (m, 2H), 7.59-7.43 (m, 3H), 6.93 (d, J=8.6 Hz, 1H), 6.67 (dd, J=2.6, 8.4 Hz, 1H), 6.40 (d, J=2.4 Hz, 1H), 3.64-3.55 (m, 2H), 3.39 (t, J=6.1 Hz, 2H), 3.10 (br s, 2H), 3.00 (br s, 2H), 2.04-1.95 (m, 2H), 1.91 (s, 3H), 1.35 (s, 2H), 1.21-1.12 (m, 2H).

### Example 269: 5-(2-(Dimethylamino)ethoxy)-2,4-dimethyl-N-(1-(naphthalen-1-yl)cyclopropyl)benzamide (Compound 405)

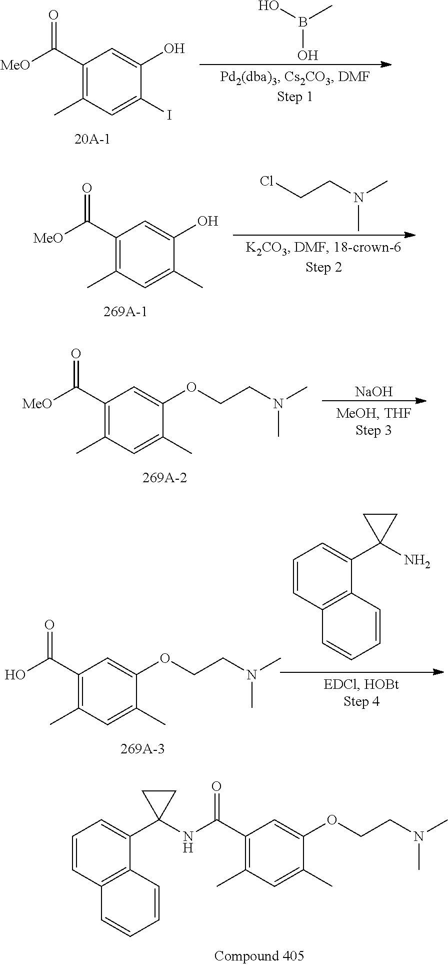

### Step 1: Methyl 5-hydroxy-2,4-dimethylbenzoate (269A-1)

To a solution of methyl 5-hydroxy-4-iodo-2-methylbenzoate (600 mg, 2.05 mmol, 1.0 eq) in DMF (20 mL) were added methylboronic acid (738 mg, 12.3 mmol, 6.0 eq), Cs₂CO₃ (2.34 g, 7.19 mmol, 3.5 eq) and Pd₂ (dba) 3 (94.1 mg, 103 μmol, 0.05 eq). The resulting mixture was degassed and purged with N₂ three times and then was stirred at 130° C. for 16 h under a N₂ atmosphere. LCMS indicated that the starting material was completely consumed, and the desired mass was detected. The reaction mixture was allowed to cool to room temperature, poured into H₂O (15 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. Methyl 5-hydroxy-2,4-dimethylbenzoate (200 mg, 333 μmol, 54% yield) was obtained as a yellow oil.

M+H⁺=181.1 (LCMS).

### Step 2: Methyl 5-(2-(dimethylamino)ethoxy)-2,4-dimethylbenzoate (269A-2)

To a solution of 2-chloro-N,N-dimethylethanamine (95.9 mg, 666 μmol, 2.0 eq, HCl salt) in DMF (10 mL) was added K₂CO₃ (230 mg, 1.66 mmol, 5.0 eq). The mixture was stirred at 20° C. for 30 min. To the resulting mixture were added methyl 5-hydroxy-2,4-dimethylbenzoate (200 mg, 333 μmol, 1.0 eq), 18-crown-6 (139 mg, 532 μmol, 1.6 eq) and KI (92.9 mg, 566 μmol, 1.7 eq). The mixture was stirred at 70° C. for 16 h. LCMS indicated that the starting material was completely consumed, and the desired mass was detected. The reaction mixture was allowed to cool to room temperature, poured into H₂O (10 mL) and extracted with EtOAc (6.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/0. Methyl 5-(2-(dimethylamino)ethoxy)-2,4-dimethyl benzoate (180 mg, 716 μmol, 50% yield) was obtained as a white solid. M+H⁺=252.1 (LCMS).

### Step 3: 5-(2-(Dimethylamino)ethoxy)-2,4-dimethylbenzoic acid (269A-3)

To a solution of methyl 5-(2-(dimethylamino)ethoxy)-2,4-dimethylbenzoate (90.0 mg, 358 μmol, 1.0 eq) in a mixture of MeOH (7.0 mL) and THF (3.5 mL) was added NaOH (2 M aqueous, 2.26 mL, 12.7 eq). The mixture was stirred at 60° C. for 16 h. LCMS indicated that the starting material was completely consumed, and the desired mass was detected. The reaction mixture was allowed to cool to room temperature, poured into H₂O (5.0 mL) and treated with HCl (1 M aqueous) to adjust the pH to 6 and the product was extracted with EtOAc (5.0 mL×3). The combined organic layers were dried over Na₂SO₄, filtered, and concentrated in vacuum to give the crude product 5-(2-(dimethylamino)ethoxy)-2,4-dimethylbenzoic acid (70.0 mg, 82% yield) as a yellow solid, which was used in the next step without any further purification. M+H⁺=238.1 (LCMS).

### Step 4: 5-(2-(Dimethylamino)ethoxy)-2,4-dimethyl-N-(1-(naphthalen-1-yl)cyclopropyl)benzamide (Compound 405)

To a solution of 5-(2-(dimethylamino)ethoxy)-2,4-dimethylbenzoic acid (70.0 mg, 295 μmol, 1.0 eq) and 1-(naphthalen-1-yl)cyclopropanamine (54.1 mg, 295 μmol, 1.0 eq) in DMF (3.5 mL) were added EDCI (84.8 mg, 442 μmol, 1.5 eq), HOBt (59.8 mg, 442 μmol, 1.5 eq) and TEA (59.7 mg, 590 μmol, 82.1 μL, 2.0 eq). The mixture was stirred at 20° C. for 16 h. LCMS indicated that the starting material was completely consumed, and the desired mass was detected. The reaction mixture was poured into H₂O (3.0 mL) and extracted with EtOAc (3.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 10%-30% B over 8 min; mobile phase A: 0.04% aqueous HCl, mobile phase B: acetonitrile). 5-(2-(Dimethylamino)ethoxy)-2,4-dimethyl-N-(1-(naphthalen-1-yl)cyclopropyl)benzamide (27.1 mg, 60.8 μmol, 21% yield, HCl salt) was obtained as a yellow solid. M+H⁺=403.1 (LCMS); ¹H NMR (400 MHZ, DMSO-d₆) δ 10.92-10.67 (m, 1H), 9.11-9.01 (m, 1H), 8.74-8.62 (m, 1H), 8.01-7.88 (m, 1H), 7.81 (s, 2H), 7.62-7.42 (m, 3H), 6.94-6.88 (m, 1H), 6.74-6.67 (m, 1H), 4.30-4.21 (m, 2H), 3.50-3.40 (m, 2H), 2.81 (d, J=4.9 Hz, 6H), 2.12 (s, 3H), 1.99-1.88 (m, 3H), 1.44-1.34 (m, 2H), 1.21-1.11 (m, 2H).

### Example 270: 5-(2-(Dimethylamino)ethoxy)-N-(1-(isoquinolin-4-yl)cyclopropyl)-2-methylbenzamide (Compound 409)

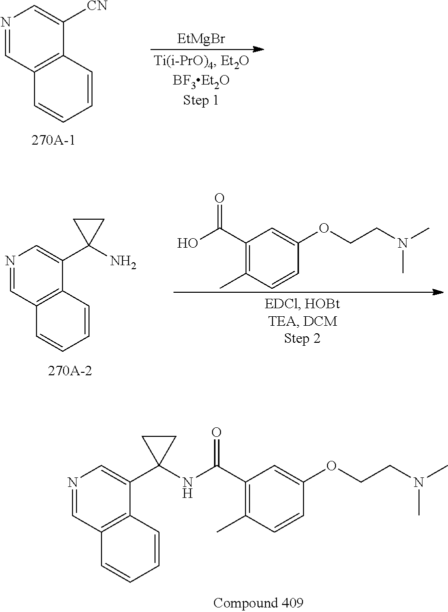 [SMILES: "EtMgBr" ; N#Cc1cncc2ccccc12 ; NC1(c2cncc3ccccc23)CC1 ; Cc1ccc(OCCN(C)C)cc1C(=O)O ; Cc1ccc(OCCN(C)C)cc1C(=O)NC1(c2cncc3ccccc23)CC1]

### Step 1: 1-(Isoquinolin-4-yl)cyclopropanamine (270A-2)

A mixture of isoquinoline-4-carbonitrile (200 mg, 1.30 mmol, 1.2 eq) in anhydrous Et₂O (12 mL) was degassed and purged with N₂ three times. The mixture was stirred at −78° C. To this mixture was added Ti(i-PrO)₄ (553 mg, 1.95 mmol, 574 μL, 1.5 eq) slowly, and then EtMgBr (3 M, 951 μL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. BF₃·Et₂O (368 mg, 2.59 mmol, 320 μL, 2.0 eq) was added slowly with no obvious temperature change.

The mixture was stirred at 20° C. for another 1 h. LCMS indicated that the starting material was completely consumed, and the desired mass was detected. The reaction mixture was added into a mixture of HCl (1 M aqueous) (10 mL) and MTBE (10 mL), and extracted with MTBE (10 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (10 mL×2). The combined organic layers were dried over Na₂SO₄, filtered, and concentrated under vacuum to give a residue which was purified by preparative TLC (DCM/MeOH=10/1, R_{f}=0.25). 1-(Isoquinolin-4-yl)cyclopropanamine (60.0 mg, 25% yield) was obtained as a yellow oil. M+H⁺=185.0 (LCMS).

### Step 2: 5-(2-(Dimethylamino)ethoxy)-N-(1-(isoquinolin-4-yl)cyclopropyl)-2-methylbenz amide (Compound 409)

To a solution of 1-(isoquinolin-4-yl)cyclopropanamine (70.0 mg, 380 μmol, 1.0 eq) and 5-(2-(dimethylamino)ethoxy)-2-methylbenzoic acid (84.8 mg, 380 μmol, 1.0 eq) in DCM (4 mL) were added TEA (115 mg, 1.14 mmol, 159 μL, 3.0 eq), EDCI (182 mg, 950 μmol, 2.5 eq) and HOBt (128 mg, 950 μmol, 2.5 eq). The mixture was stirred at 20° C. for 14 h. LCMS indicated that the starting material was completely consumed, and the desired mass was detected. The reaction mixture was poured into H₂O (10 mL) and extracted with DCM (10 mL×2). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 1%-35% B over 8 min; mobile phase A: 0.2% aqueous FA, mobile phase B: acetonitrile). 5-(2-(Dimethylamino)ethoxy)-N-(1-(isoquinolin-4-yl)cyclopropyl)-2-methylbenzamide (38.3 mg, 85.6 μmol, 23% yield, FA salt) was obtained as a pink solid. M+H⁺=390.1 (LCMS); ¹H NMR (400 MHZ, DMSO-d₆) δ 9.23 (s, 1H), 9.14 (s, 1H), 8.75 (s, 1H), 8.64 (d, J=8.4 Hz, 1H), 8.19-8.09 (m, 2H), 7.85 (ddd, J=1.3, 7.0, 8.4 Hz, 1H), 7.73-7.62 (m, 1H), 7.03 (d, J=8.5 Hz, 1H), 6.84 (dd, J=2.8, 8.4 Hz, 1H), 6.63 (d, J=2.6 Hz, 1H), 3.97 (t, J=5.8 Hz, 2H), 2.62 (t, J=5.7 Hz, 2H), 2.22 (s, 6H), 1.94 (s, 3H), 1.40-1.33 (m, 2H), 1.26-1.19 (m, 2H).

### Example 271: N-(1-(9H-Carbazol-4-yl)cyclopropyl)-5-(2-(dimethylamino)ethoxy)-2-methylbenzamide (Compound 415)

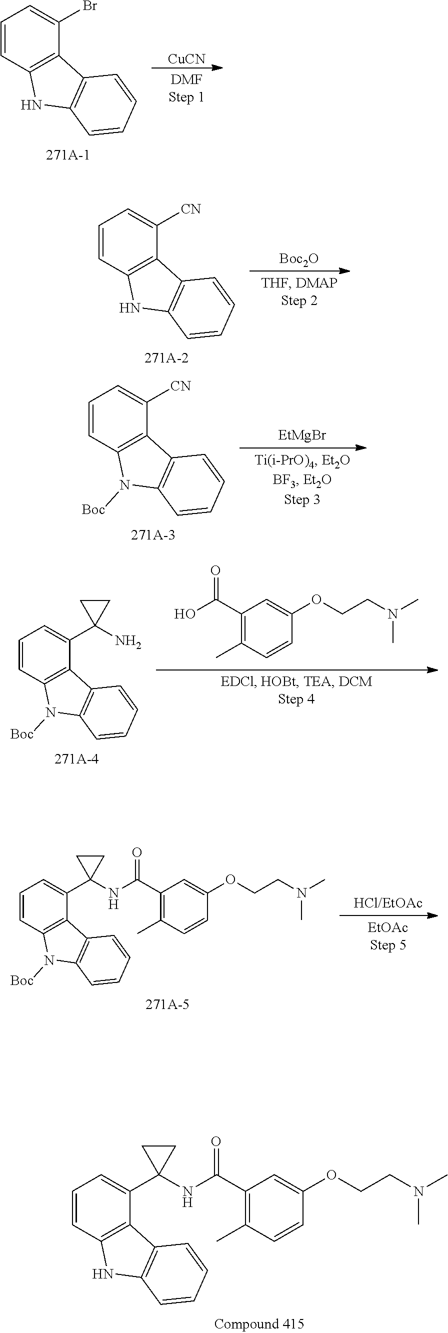

### Step 1: 9H-Carbazole-4-carbonitrile (271A-2)

To a solution of 4-bromo-9H-carbazole (1.00 g, 4.06 mmol, 1.0 eq) in DMF (10 mL) was added CuCN (582 mg, 6.50 mmol, 1.42 mL, 1.6 eq) at 20° C. The mixture was stirred at 150° C. for 18 h. TLC indicated that the starting material was completely consumed. The reaction mixture was allowed to cool to room temperature, poured into H₂O (10 mL) and extracted with EtOAc (10 mL×3). The combined organic layers were washed with brine (10 mL×3), dried over Na₂SO₄, filtered, and concentrated under vacuum to give a residue which was purified by flash silica gel chromatography using a gradient of EtOAc/petroleum ether from 0/1 to 1/1. 9H-Carbazole-4-carbonitrile (600 mg, 3.12 mmol, 77% yield) was obtained as a white solid. M−H⁺=191.0 (LCMS).

### Step 2: tert-Butyl 4-cyano-9H-carbazole-9-carboxylate (271A-3)

To a solution of 9H-carbazole-4-carbonitrile (400 mg, 2.08 mmol, 1.0 eq) in THF (5.0 mL) were added DMAP (280 mg, 2.29 mmol, 1.1 eq) and Boc₂O (500 mg, 2.29 mmol, 526 μL, 1.1 eq). The mixture was stirred at 20° C. for 16 h. TLC indicated that the starting material was completely consumed. The reaction mixture was poured into H₂O (5.0 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. tert-Butyl 4-cyano-9H-carbazole-9-carboxylate (400 mg, 1.37 mmol, 66% yield) was obtained as a white solid. M+H⁺=293.1 (LCMS).

### Step 3: tert-Butyl 4-(1-aminocyclopropyl)-9H-carbazole-9-carboxylate (271A-4)

A mixture of tert-butyl 4-cyano-9H-carbazole-9-carboxylate (100 mg, 342 μmol, 1.0 eq) in anhydrous Et₂O (7.5 mL) was degassed and purged with N₂ three times. The mixture was stirred at −78° C. To this mixture was added Ti(i-PrO)₄ (146 mg, 513 μmol, 151 μL, 1.5 eq) slowly and then EtMgBr (3 M in Et₂O, 251 μL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. BF₃·Et₂O (97.1 mg, 684 μmol, 84.4 μL, 2.0 eq) was added slowly with no obvious temperature change. The mixture was stirred at 20° C. for another 1 h. TLC indicated that the starting material was completely consumed, and a main spot was detected. The reaction mixture was added into a mixture of HCl (1 M aqueous) (8.0 mL) and MTBE (8.0 mL) and the mixture was extracted with MTBE (8.0 mL×4). The aqueous layer was basified to pH 8 using NaOH (2 M aqueous) and a precipitate was formed. The mixture was filtered through a pad of Celite and the slurry was washed with DCM several times. The combined filtrate was extracted with DCM (10 mL×4). The combined organic layers were dried over Na₂SO₄, filtered, and concentrated under vacuum to give a residue which was purified by flash silica gel chromatography using a gradient of EtOAc/petroleum ether from 0/1 to 1/1. tert-Butyl 4-(1-aminocyclopropyl)-9H-carbazole-9-carboxylate (30.0 mg, 93.1 μmol, 27% yield) was obtained as a white solid. M+H⁺=323.1 (LCMS); ¹H NMR (400 MHZ, DMSO-d₆) δ 8.49 (d, J=7.7 Hz, 1H), 8.31 (d, J=8.2 Hz, 1H), 8.21-8.17 (m, 1H), 7.57-7.52 (m, 1H), 7.48-7.42 (m, 2H), 7.40-7.35 (m, 1H), 1.71 (s, 9H), 1.11 (br s, 2H), 0.97 (br s, 2H).

### Step 4: tert-Butyl 4-(1-(5-(2-(dimethylamino)ethoxy)-2-methylbenzamido)cyclopropyl)-9H-carbazole-9-carboxylate (271A-5)

To a solution of 5-(2-(dimethylamino)ethoxy)-2-methylbenzoic acid (49.9 mg, 223 μmol, 1.2 eq) in DCM (2.0 mL) were added TEA (56.5 mg, 558 μmol, 77.7 μL, 3.0 eq), EDCI (53.5 mg, 279 μmol, 1.5 eq), HOBt (37.7 mg, 279 μmol, 1.5 eq) and tert-butyl 4-(1-aminocyclopropyl)-9H-carbazole-9-carboxylate (60.0 mg, 186 μmol, 1.0 eq). The mixture was stirred at 20° C. for 3 h. TLC indicated that the starting material was completely consumed. The reaction mixture was poured into H₂O (5.0 mL) and extracted with EtOAc (5.0 mL×3). The combined organic layers were dried over Na₂SO₄, filtered, and concentrated under vacuum to give a residue which was purified by preparative TLC (DCM/MeOH=10/1, R_{f}=0.4). tert-Butyl 4-(1-(5-(2-(dimethylamino)ethoxy)-2-methylbenzamido)cyclopropyl)-9H-carbazole-9-carboxylate (30.0 mg, 56.9 μmol, 31% yield) was obtained as a white solid. M+H⁺=528.2 (LCMS).

### Step 5: N-(1-(9H-Carbazol-4-yl)cyclopropyl)-5-(2-(dimethylamino)ethoxy)-2-methylbenz amide (Compound 415)

To a solution of tert-butyl 4-(1-(5-(2-(dimethylamino)ethoxy)-2-methylbenzamido)cyclopropyl)-9H-carbazole-9-carboxylate (30.0 mg, 56.9 μmol, 1.0 eq) in EtOAc (1.0 mL) was added HCl/EtOAc (4 M, 3.0 mL). The mixture was stirred at 20° C. for 1 h. LCMS indicated that the starting material was completely consumed, and the desired mass was detected. The reaction mixture was concentrated under vacuum to give a residue which was purified by preparative HPLC (Phenomenex Luna column (80×30 mm, 3 μm); flow rate: 25 mL/min; gradient: 20%-50% B over 8 min; mobile phase A: 0.04% aqueous HCl, mobile phase B: acetonitrile). N-(1-(9H-Carbazol-4-yl)cyclopropyl)-5-(2-(dimethylamino)ethoxy)-2-methylbenzamide (6.50 mg, 15.2 μmol, 27% yield, HCl salt) was obtained as a white solid. M+H⁺=428.2 (LCMS); ¹H NMR (400 MHZ, DMSO-d₆) δ 11.32 (s, 1H), 10.14-10.03 (m, 1H), 8.96 (s, 1H), 8.62 (d, J=7.9 Hz, 1H), 7.54 (d, J=7.3 Hz, 1H), 7.50-7.46 (m, 1H), 7.42-7.36 (m, 2H), 7.34-7.29 (m, 1H), 7.16 (t, J=7.3 Hz, 1H), 7.07 (d, J=8.5 Hz, 1H), 6.90-6.85 (m, 1H), 6.62 (d, J=2.6 Hz, 1H), 4.15 (br t, J=4.8 Hz, 2H), 3.42-3.36 (m, 2H), 2.78-2.73 (m, 6H), 1.99 (s, 3H), 1.44 (br s, 2H), 1.23 (br s, 2H).

### Example 272: 5-(2-(Dimethylamino)ethoxy)-N-(1-(7-hydroxynaphthalen-1-yl)cyclopropyl)-2-methylbenzamide (Compound 417)

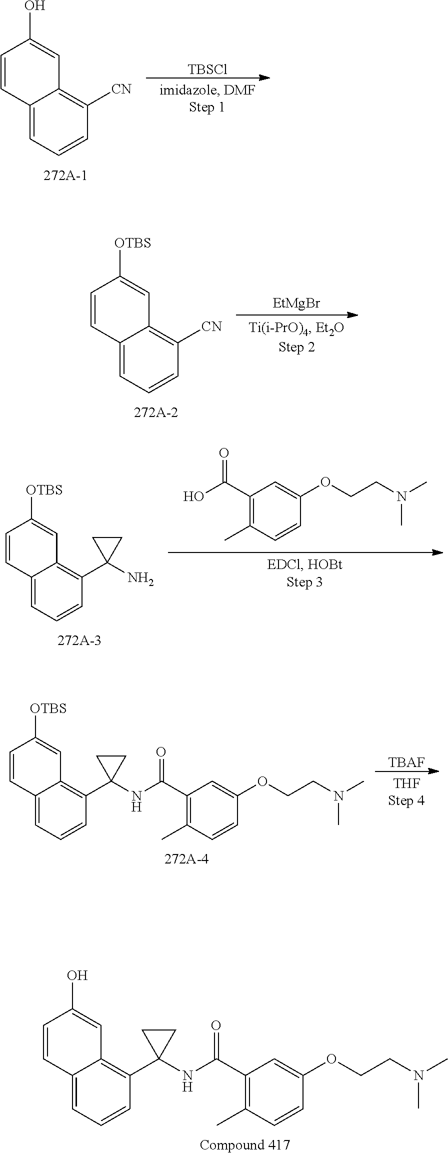

### Step 1: 7-((tert-Butyldimethylsilyl)oxy)-1-naphthonitrile (272A-2)

To a solution of 7-hydroxy-1-naphthonitrile (500 mg, 2.96 mmol, 1.0 eq) in DMF (5.0 mL) were added TBSCl (535 mg, 3.55 mmol, 435 μL, 1.2 eq) and imidazole (402 mg, 5.91 mmol, 2.0 eq). The resulting mixture was stirred at 20° C. for 16 h. LCMS indicated that the starting material was completely consumed, and the desired mass was detected. The reaction mixture was poured into H₂O (20 mL) and extracted with EtOAc (10 mL×4). The combined organic layers were washed with brine (10 mL×2), dried over Na₂SO₄, filtered, and concentrated under vacuum to give a residue which was purified by flash silica gel chromatography using a gradient of EtOAc/petroleum ether from 0/1 to 1/3. 7-((tert-Butyldimethylsilyl)oxy)-1-naphthonitrile (770 mg, 2.72 mmol, 92% yield) was obtained as a yellow gum. M+H⁺=284.1 (LCMS); ¹H NMR (400 MHZ, CDCl₃) δ 8.00 (d, J=8.3 Hz, 1H), 7.87 (d, J=7.1 Hz, 1H), 7.82 (d, J=8.9 Hz, 1H), 7.58 (d, J=2.1 Hz, 1H), 7.38 (t, J=7.8 Hz, 1H), 7.21 (dd, J=2.3, 8.8 Hz, 1H), 1.04 (s, 9H), 0.31 (s, 6H).

### Step 2: 1-(7-((tert-Butyldimethylsilyl)oxy) naphthalen-1-yl)cyclopropanamine (272A-3)

A mixture of 7-((tert-butyldimethylsilyl)oxy)-1-naphthonitrile (300 mg, 1.06 mmol, 1.0 eq) in Et₂O (20 mL) was degassed and purged with N₂ three times. To this mixture was added Ti(i-PrO)₄ (451 mg, 1.59 mmol, 469 μL, 1.5 eq) slowly at −78° C. and then EtMgBr (3 M in Et₂O, 776 μL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. Then BF₃·Et₂O (300 mg, 2.12 mmol, 261 μL, 2.0 eq) was added slowly with no obvious temperature change. The mixture was stirred at 20° C. for another 1 h. TLC indicated that the starting material was completely consumed. The reaction mixture was added into a mixture of HCl (1 M aqueous) (10 mL) and MTBE (10 mL), and extracted with MTBE (10 mL×2). The aqueous layer was basified to pH 8 using NaOH (2 M aqueous), and a precipitate was formed. The mixture was filtered through a pad of Celite and the slurry was washed with DCM several times. The combined filtrate was extracted with DCM (10 mL×2). The combined organic layers were dried over Na₂SO₄, filtered, and concentrated under vacuum to give a residue which was purified by flash silica gel chromatography using a gradient of EtOAc/petroleum ether from 0/1 to 1/0. 1-(7-((tert-Butyldimethylsilyl)oxy) naphthalen-1-yl)cyclopropanamine (220 mg, 702 μmol, 66% yield) was obtained as a yellow gum. ¹H NMR (400 MHZ, CDCl₃) δ 7.75 (br d, J=8.9 Hz, 2H), 7.68 (br d, J=7.0 Hz, 1H), 7.60 (s, 1H), 7.32-7.28 (m, 1H), 7.10 (dd, J=1.8, 8.8 Hz, 1H), 1.10 (s, 2H), 1.05-0.76 (m, 11H), 0.24 (s, 6H).
Step 3: N-(1-(7-((tert-Butyldimethylsilyl)oxy) naphthalen-1-yl)cyclopropyl)-5-(2-(dimethylamino)ethoxy)-2-methylbenzamide (272A-4)

To a solution of 1-(7-((tert-butyldimethylsilyl)oxy) naphthalen-1-yl)cyclopropanamine (220 mg, 702 μmol, 1.0 eq) and 5-(2-(dimethylamino)ethoxy)-2-methylbenzoic acid (157 mg, 702 μmol, 1.0 eq) in DMF (10 mL) were added TEA (213 mg, 2.11 mmol, 293 μL, 3.0 eq), EDCI (161 mg, 842 μmol, 1.2 eq) and HOBt (114 mg, 842 μmol, 1.2 eq). The mixture was stirred at 20° C. for 16 h. LCMS indicated that the starting material was completely consumed, and the desired mass was detected. The reaction mixture was poured into H₂O (10 mL) and extracted with EtOAc (10 mL×5). The combined organic layers were washed with brine (10 mL×2), dried over Na₂SO₄, filtered, and concentrated under vacuum to give a residue which was purified by flash silica gel chromatography using a gradient of EtOAc/petroleum ether from 0/1 to 1/0. N-(1-(7-((tert-Butyldimethylsilyl)oxy) naphthalen-1-yl)cyclopropyl)-5-(2-(dimethylamino)ethoxy)-2-methylbenzamide (140 mg, 270 μmol, 38% yield) was obtained as a yellow gum.

### Step 4: 5-(2-(Dimethylamino)ethoxy)-N-(1-(7-hydroxynaphthalen-1-yl)cyclopropyl)-2-methylbenzamide (Compound 417)

To a solution of N-(1-(7-((tert-butyldimethylsilyl)oxy) naphthalen-1-yl)cyclopropyl)-5-(2-(dimethylamino)ethoxy)-2-methylbenzamide (130 mg, 251 μmol, 1.0 eq) in THF (10 mL) was added TBAF (1 M in THF, 752 μL, 3.0 eq) at 0° C. The mixture was stirred at 20° C. for 3 h. LCMS indicated that the starting material was completely consumed, and the desired mass was detected. The reaction mixture was poured into H₂O (10 mL) and extracted with EtOAc (5.0 mL×5), concentrated under vacuum to give a residue which was purified by preparative HPLC (Phenomenex Luna C18 column (80×30 mm, 3 μm); flow rate: 25 mL/min; gradient: 10%-50% B over 8 min; mobile phase A: 0.04% aqueous HCl, mobile phase B: acetonitrile). 5-(2-(Dimethylamino)ethoxy)-N-(1-(7-hydroxynaphthalen-1-yl)cyclopropyl)-2-methylbenzamide (17.9 mg, 40.6 μmol, 16% yield, HCl salt) was obtained as a brown solid. M+H⁺=405.1 (LCMS); ¹H NMR (400 MHZ, DMSO-d₆) § 10.08-9.89 (m, 1H), 9.77-9.51 (m, 1H), 9.05 (s, 1H), 7.84 (d, J=2.1 Hz, 1H), 7.79-7.61 (m, 3H), 7.27-7.04 (m, 3H), 6.90 (dd, J=2.7, 8.3 Hz, 1H), 6.69 (d, J=2.6 Hz, 1H), 4.25 (br t, J=4.9 Hz, 2H), 3.44 (q, J=5.0 Hz, 2H), 2.80 (d, J=4.9 Hz, 6H), 2.00 (s, 3H), 1.32 (br s, 2H), 1.12 (br s, 2H).

### Example 273: 5-(2-(Dimethylamino)ethoxy)-N-(1-(3-(ethylthio) naphthalen-1-yl)cyclopropyl)-2-methylbenzamide (Compound 408)

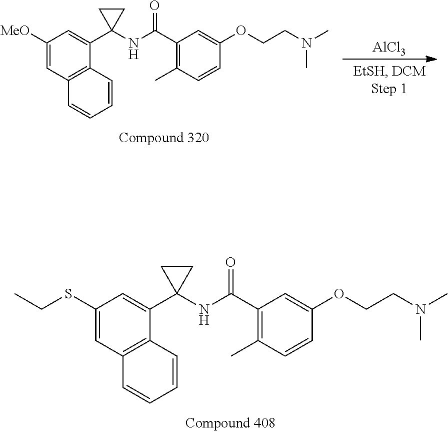

### Step 1: 5-(2-(Dimethylamino)ethoxy)-N-(1-(3-(ethylthio) naphthalen-1-yl)cyclopropyl)-2-methylbenzamide (Compound 408)

To a solution of EtSH (5.04 g, 81.1 mmol, 6.0 mL, 67.9 eq) in DCM (25 mL) was added AlCl₃ (956 mg, 7.17 mmol, 392 μL, 6.0 eq) at 0° C., then 5-(2-(dimethylamino)ethoxy)-N-(1-(3-methoxynaphthalen-1-yl)cyclopropyl)-2-methylbenzamide (500 mg, 1.19 mmol, 1.0 eq) in DCM (5.0 mL) was added at 0° C. dropwise. The resulting mixture was stirred at the same temperature for 10 min and warmed to 20° C. The resulting mixture was stirred at the same temperature for 2 h. LCMS indicated that the starting material was completely consumed, and the desired mass was detected. The reaction mixture was poured into H₂O (30 mL) and extracted with DCM (30 mL×3). The combined organic layers were dried over Na₂SO₄, filtered, and concentrated under vacuum to give a residue which was purified by preparative HPLC (Phenomenex Luna C18 column (200×40 mm, 10 μm); flow rate: 50 mL/min; gradient: 10%-50% B over 8 min; mobile phase A: 0.2% aqueous FA, mobile phase B: acetonitrile). 5-(2-(Dimethylamino)ethoxy)-N-(1-(3-(ethylthio) naphthalen-1-yl)cyclopropyl)-2-methyl benzamide (250 mg, 535 μmol, 45% yield) was obtained as a brown gum. M+H⁺=449.1 (LCMS); ¹H NMR (400 MHz, DMSO-d₆) δ 9.09 (s, 1H), 8.58 (dd, J=3.4, 6.2 Hz, 1H), 8.19 (s, 1H), 7.94-7.79 (m, 1H), 7.75-7.66 (m, 2H), 7.56-7.39 (m, 2H), 7.03 (d, J=8.4 Hz, 1H), 6.84 (dd, J=2.6, 8.4 Hz, 1H), 6.61 (d, J=2.6 Hz, 1H), 3.97 (t, J=5.7 Hz, 2H), 3.10 (q, J=7.3 Hz, 2H), 2.63 (t, J=5.7 Hz, 2H), 2.23 (s, 6H), 1.97 (s, 3H), 1.39-1.28 (m, 5H), 1.18 (br s, 2H).

### Example 274: (S)—N-(1-(3-Methoxynaphthalen-1-yl)cyclopropyl)-2-methyl-5-(pyrrolidin-2-ylmethoxy)benzamide (Compound 419)

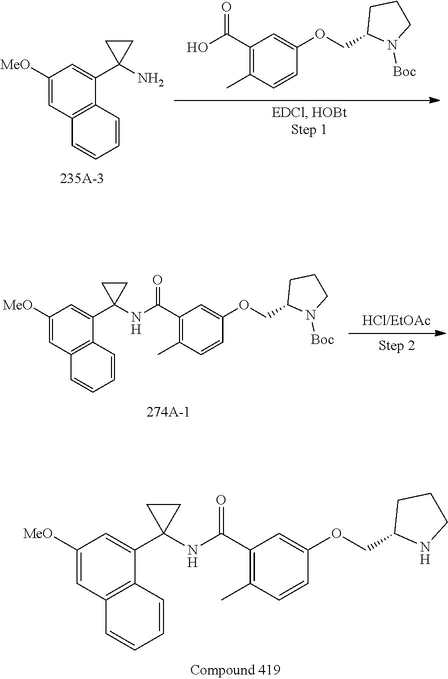 [SMILES: COc1cc(C2(N)CC2)c2ccccc2c1 ; Cc1ccc(OC[C@@H]2CCCN2C(=O)OC(C)(C)C)cc1C(=O)O ; COc1cc(C2(NC(=O)c3cc(OC[C@@H]4CCCN4C(=O)OC(C)(C)C)ccc3C)CC2)c2ccccc2c1 ; COc1cc(C2(NC(=O)c3cc(OC[C@@H]4CCCN4)ccc3C)CC2)c2ccccc2c1]

### Step 1: (S)-tert-Butyl 2-((3-((1-(3-methoxynaphthalen-1-yl)cyclopropyl)carbamoyl)-4-methylphenoxy)methyl)pyrrolidine-1-carboxylate (274A-1)

To a solution of 1-(3-methoxynaphthalen-1-yl)cyclopropanamine (63.6 mg, 298 μmol, 1.0 eq) and(S)-5-((1-(tert-butoxycarbonyl)pyrrolidin-2-yl)methoxy)-2-methylbenzoic acid (100 mg, 298 μmol, 1.0 eq) in DCM (5.0 mL) were added TEA (90.5 mg, 894 μmol, 125 μL, 3.0 eq), EDCI (143 mg, 745 μmol, 2.5 eq) and HOBt (101 mg, 745 μmol, 2.5 eq). The mixture was stirred at 20° C. for 16 h. LCMS indicated that the starting material was completely consumed, and the desired mass was detected. The reaction mixture was poured into H₂O (5.0 mL) and extracted with EtOAc (3.0 mL×2). The combined organic layers were dried over Na₂SO₄, filtered, and concentrated under vacuum to give a residue which was purified by flash silica gel chromatography using a gradient of EtOAc/petroleum ether from 1/10 to 1/2. (S)-tert-Butyl 2-((3-((1-(3-methoxynaphthalen-1-yl)cyclopropyl)carbamoyl)-4-methylphenoxy)methyl) pyrrolidine-1-carboxylate (150 mg, 283 μmol, 95% yield) was obtained as a colorless oil. M+H⁺=531.3 (LCMS).

### Step 2: (S)—N-(1-(3-Methoxynaphthalen-1-yl)cyclopropyl)-2-methyl-5-(pyrrolidin-2-ylmethoxy)benzamide (Compound 419)

To a solution of(S)-tert-butyl 2-((3-((1-(3-methoxynaphthalen-1-yl)cyclopropyl)carbamoyl)-4-methylphenoxy)methyl)pyrrolidine-1-carboxylate (150 mg, 283 μmol, 1.0 eq) in EtOAc (2.0 mL) was added HCl/EtOAc (4 M, 2.0 mL). The mixture was stirred at 20° C. for 1 h. LCMS indicated that the starting material was completely consumed, and the desired mass was detected. The mixture was concentrated under vacuum to give a residue which was purified by preparative HPLC (Phenomenex Luna C18 column (80×30 mm, 3 μm); flow rate: 25 mL/min; gradient: 5%-50% B over 8 min; mobile phase A: 0.04% aqueous HCl, mobile phase B: acetonitrile). (S)—N-(1-(3-Methoxynaphthalen-1-yl)cyclopropyl)-2-methyl-5-(pyrrolidin-2-ylmethoxy)benzamide (80.3 mg, 172 μmol, 61% yield, HCl salt) was obtained as a white solid. M+H⁺=431.1 (LCMS); ¹H NMR (400 MHz, DMSO-d₆) δ 9.55 (br d, J=4.0 Hz, 1H), 9.14 (s, 1H), 8.97 (br d, J=3.9 Hz, 1H), 8.54 (d, J=8.2 Hz, 1H), 7.83 (d, J=8.1 Hz, 1H), 7.51-7.35 (m, 3H), 7.24 (d, J=2.3 Hz, 1H), 7.08 (d, J=8.4 Hz, 1H), 6.89 (dd, J=2.6, 8.4 Hz, 1H), 6.67 (d, J=2.6 Hz, 1H), 4.20-4.11 (m, 1H), 4.09-4.00 (m, 1H), 3.87 (s, 4H), 3.23-3.11 (m, 2H), 2.12-2.01 (m, 1H), 1.99-1.96 (m, 3H), 1.95-1.82 (m, 2H), 1.72-1.61 (m, 1H), 1.33 (br s, 2H), 1.16 (br s, 2H).

### Example 275: 5-(2-(Dimethylamino)ethoxy)-2-methyl-N-(1-(3-(thiophen-2-yl)naphthalen-1-yl)cyclopropyl)benzamide (Compound 420)

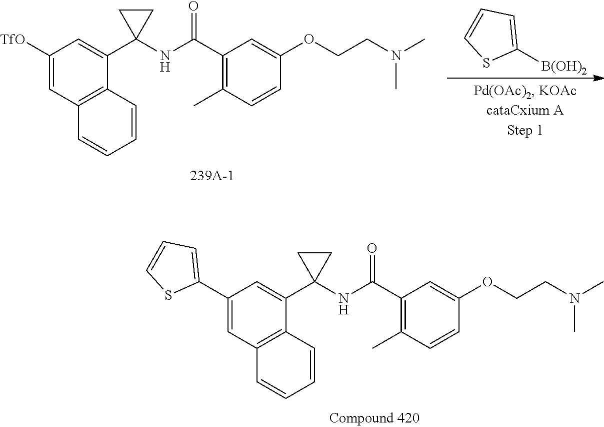

### Step 1: 5-(2-(Dimethylamino)ethoxy)-2-methyl-N-(1-(3-(thiophen-2-yl)naphthalen-1-yl)cyclopropyl)benzamide (Compound 420)

To a solution of 4-(1-(5-(2-(dimethylamino)ethoxy)-2-methylbenzamido)cyclopropyl) naphthalen-2-yl trifluoromethanesulfonate (50.0 mg, 93.2 μmol, 1.0 eq) and thiophen-2-ylboronic acid (14.3 mg, 112 μmol, 1.2 eq) in DMSO (5.0 mL) were added Pd(OAc) 2 (2.09 mg, 9.32 μmol, 0.1 eq), KOAc (27.4 mg, 280 μmol, 3.0 eq) and bis(1-adamantyl)-butyl-phosphane (6.68 mg, 18.6 μmol, 0.2 eq). The mixture was degassed and purged with N₂ three times, and then the mixture was stirred at 80° C. for 16 h under a N₂ atmosphere. LCMS indicated that the starting material was completely consumed, and the desired mass was detected. The reaction mixture was allowed to cool to room temperature, poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Luna C18 column (100×30 mm, 5 μm); flow rate: 25 mL/min; gradient: 25%-55% B over 10 min; mobile phase A: 0.04% aqueous HCl, mobile phase B: acetonitrile). 5-(2-(Dimethylamino)ethoxy)-2-methyl-N-(1-(3-(thiophen-2-yl)naphthalen-1-yl)cyclopropyl)benzamide (7.10 mg, 14.0 μmol, 15% yield, HCl salt) was obtained as a white solid. M+H⁺=371.1 (LCMS); ¹H NMR (400 MHZ, DMSO-d₆) δ 10.16-10.05 (m, 1H), 9.19 (s, 1H), 8.66-8.58 (m, 1H), 8.14-8.07 (m, 2H), 8.03-7.95 (m, 1H), 7.68-7.65 (m, 1H), 7.63 (d, J=5.0 Hz, 1H), 7.60-7.50 (m, 2H), 7.21 (dd, J=3.7, 5.0 Hz, 1H), 7.09 (d, J=8.4 Hz, 1H), 6.90 (dd, J=2.6, 8.3 Hz, 1H), 6.69 (d, J=2.6 Hz, 1H), 4.24 (t, J=4.8 Hz, 2H), 3.47-3.39 (m, 2H), 2.79 (d, J=4.9 Hz, 6H), 1.98 (s, 3H), 1.39 (br s, 2H), 1.27 (br s, 2H).

### Example 276: 5-Amino-2-methyl-N-(1-(naphthalen-1-yl) prop-2-yn-1-yl)benzamide (Compound 459)

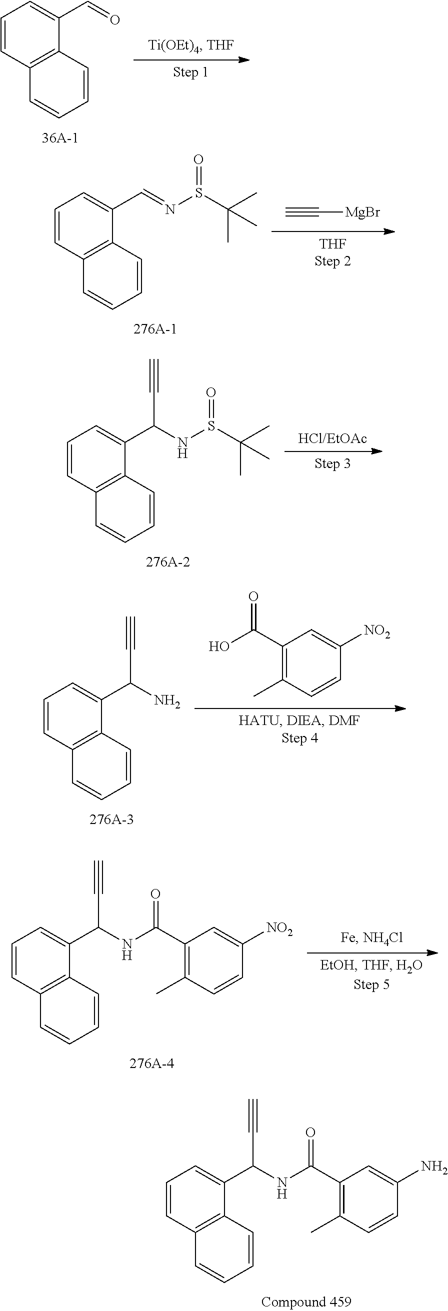

### Step 1: (E)-2-Methyl-N-(naphthalen-1-ylmethylene) propane-2-sulfinamide (276A-1)

To a mixture of naphthalene-1-carbaldehyde (2.00 g, 12.8 mmol, 1.74 mL, 1.0 eq) and 2-methylpropane-2-sulfinamide (2.33 g, 19.2 mmol, 1.5 eq) in THF (80 mL) was added Ti(OEt)₄ (5.26 g, 23.0 mmol, 4.78 mL, 1.8 eq) at 25° C., the reaction mixture was stirred at 60° C. for 16 h. LCMS indicated that the starting material was completely consumed, and the desired mass was detected. The reaction mixture was allowed to cool to room temperature, poured into H₂O (50 ml) and the mixture was filtered. The filtrate was purified by flash silica gel chromatography using a gradient of EtOAc/petroleum ether from 1/0 to 3/1. (E)-2-Methyl-N-(1-naphthylmethylene) propane-2-sulfinamide (3.00 g, 11.6 mmol, 90% yield) was obtained as a yellow oil. M+H⁺=260.2 (LCMS); ¹H NMR (400 MHZ, CD₃OD) δ 9.12 (s, 1H), 9.03 (d, J=8.4 Hz, 1H), 8.16-8.07 (m, 2H), 8.01 (d, J=7.6 Hz, 1H), 7.74-7.56 (m, 3H), 1.33 (s, 9H).

### Step 2: 2-Methyl-N-(1-(naphthalen-1-yl) prop-2-yn-1-yl) propane-2-sulfinamide (276A-2)

To a mixture of (E)-2-methyl-N-(1-naphthylmethylene) propane-2-sulfinamide (3.00 g, 11.6 mmol, 1.0 eq) in THF (60 mL) was added bromo (ethynyl)magnesium (0.5 M in THF, 46.3 mL, 2.0 eq) at 0° C. The reaction mixture was stirred at 25° C. for 16 h under a N₂ atmosphere. LCMS indicated that the starting material was completely consumed, and the desired mass was detected. The reaction mixture was poured into saturated aqueous NH₄Cl (50 mL) and extracted with EtOAc (100 mL×3). The combined organic layers were concentrated under vacuum to give a residue which was purified by flash silica gel chromatography using a gradient of EtOAc/petroleum ether from 1/0 to 1/1. 2-Methyl-N-[1-(1-naphthyl) prop-2-ynyl]propane-2-sulfinamide (1.00 g, 3.50 mmol, 30% yield) was obtained as a brown oil. M+H⁺=286.0 (LCMS); ¹H NMR (400 MHZ, CDCl₃) δ 8.24 (d, J=8.0 Hz, 1H), 7.99-7.84 (m, 4H), 7.65-7.47 (m, 4H), 5.92 (dd, J=2.0, 6.8 Hz, 1H), 2.75 (d, J=2.4 Hz, 1H), 1.22 (s, 9H).

### Step 3: 1-(Naphthalen-1-yl) prop-2-yn-1-amine (276A-3)

To a mixture of 2-methyl-N-[1-(1-naphthyl) prop-2-ynyl]propane-2-sulfinamide (500 mg, 1.75 mmol, 1.0 eq) in EtOAc (1.0 mL) was added HCl/EtOAc (4 M, 4.0 mL). The mixture was stirred at 25° C. for 1 h. LCMS indicated that the starting material was completely consumed, and the desired mass was detected. The reaction mixture was concentrated under vacuum to give a crude product 1-(1-naphthyl) prop-2-yn-1-amine (250 mg, HCl salt) as a brown solid. M+H⁺=182.2 (LCMS).

### Step 4: 2-Methyl-N-(1-(naphthalen-1-yl) prop-2-yn-1-yl)-5-nitrobenzamide (276A-4)

To a solution of 1-(1-naphthyl) prop-2-yn-1-amine (100 mg, 459 μmol, 1.0 eq, HCl salt) and 2-methyl-5-nitro-benzoic acid (83.2 mg, 459 μmol, 1.0 eq) in DMF (2.0 mL) were added HATU (262 mg, 689 μmol, 1.5 eq) and DIEA (178 mg, 1.38 mmol, 240 μL, 3.0 eq) at 25° C., the reaction mixture was stirred at 25° C. for 1 h. LCMS indicated that the starting material was completely consumed, and the desired mass was detected. The reaction mixture was poured into H₂O (50 ml) and the mixture was filtered. The filter cake was purified by preparative TLC (EtOAc/petroleum ether=1/1, R_{f}=0.2). 2-Methyl-N-[1-(1-naphthyl) prop-2-ynyl]-5-nitro-benzamide (60.0 mg, 174 μmol, 38% yield) was obtained as a white solid. M+H⁺=182.2 (LCMS).

### Step 5: 5-Amino-2-methyl-N-(1-(naphthalen-1-yl) prop-2-yn-1-yl)benzamide (Compound 459)

To a mixture of 2-methyl-N-[1-(1-naphthyl) prop-2-ynyl]-5-nitro-benzamide (45.0 mg, 130 μmol, 1.0 eq) in a mixture of THF (600 μL), EtOH (600 μL) and H₂O (200 μL) were added Fe (36.5 mg, 653 μmol, 5.0 eq) and NH₄Cl (34.9 mg, 653 μmol, 5.0 eq) at 25° C. The reaction mixture was stirred at 80° C. for 2 h under a N₂ atmosphere. LCMS indicated that the starting material was completely consumed, and the desired mass was detected. The reaction mixture was allowed to cool to room temperature. The mixture was filtered, and the filtrate was concentrated under vacuum to give a residue which was purified by preparative HPLC (Phenomenex Luna C18 (75×30 mm, 3 μm); flow rate: 25 mL/min; gradient: 15%-50% B over 8 min; mobile phase A: 10 mM aqueous NH₄HCO₃, mobile phase B: acetonitrile). 5-Amino-2-methyl-N-[1-(1-naphthyl) prop-2-ynyl]benzamide (9.80 mg, 31.2 μmol, 24% yield) was obtained as a brown solid. M+H⁺=315.1 (LCMS); ¹H NMR (400 MHZ, CDCl₃) δ 8.12 (d, J=8.4 Hz, 1H), 7.91 (d, J=7.2 Hz, 1H), 7.81 (dd, J=8.4, 12.0 Hz, 2H), 7.55-7.37 (m, 3H), 6.88 (d, J=8.0 Hz, 1H), 6.81-6.72 (m, 1H), 6.63-6.52 (m, 2H), 6.23-5.98 (m, 1H), 2.55 (d, J=2.4 Hz, 1H), 2.22 (s, 3H).

### Example 277: 2-Methyl-5-((1-methylazetidin-2-yl)methoxy)-N-(1-(naphthalen-1-yl) prop-2-yn-1-yl)benzamide (Compound 448)

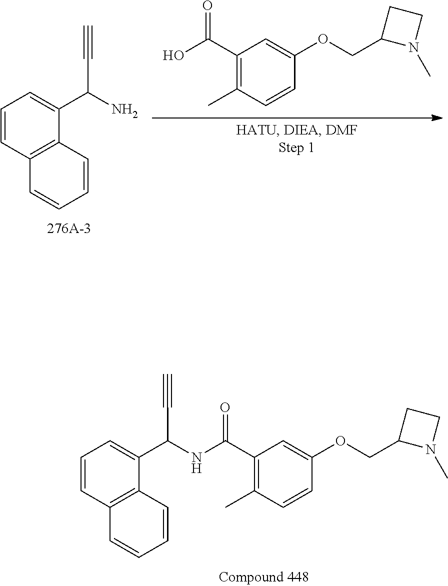 [SMILES: C#CC(N)c1cccc2ccccc12 ; Cc1ccc(OCC2CCN2C)cc1C(=O)O ; C#CC(NC(=O)c1cc(OCC2CCN2C)ccc1C)c1cccc2ccccc12]

### Step 1: 2-Methyl-5-((1-methylazetidin-2-yl)methoxy)-N-(1-(naphthalen-1-yl) prop-2-yn-1-yl)benzamide (Compound 448)

To a mixture of 1-(1-naphthyl) prop-2-yn-1-amine (50.0 mg, 229 μmol, 1.0 eq, HCl salt) and 2-methyl-5-[(1-methylazetidin-2-yl)methoxy]benzoic acid (54.0 mg, 229 μmol, 1.0 eq) in DMF (1.0 mL) were added DIEA (89.1 mg, 689 μmol, 120 μL, 3.0 eq) and HATU (131 mg, 345 μmol, 1.5 eq) at 25° C., the reaction mixture was stirred at 25° C. for 1 h. LCMS indicated that the starting material was completely consumed. The reaction was concentrated under vacuum to give a residue which was purified by preparative HPLC (Waters Xbridge Prep OBD C18 (150×40 mm, 10 μm); flow rate: 25 mL/min; gradient: 35%-65% B over 8 min; mobile phase A: 10 mM aqueous NH₄HCO₃, mobile phase B: acetonitrile). 2-Methyl-5-[(1-methylazetidin-2-yl)methoxy]-N-[1-(1-naphthyl) prop-2-ynyl]benzamide (34.7 mg, 84.1 μmol, 37% yield) was obtained as a brown solid. M+H⁺=399.2 (LCMS); ¹H NMR (400 MHZ, CDCl₃) δ 8.13 (d, J=8.4 Hz, 1H), 7.92 (d, J=7.2 Hz, 1H), 7.86-7.76 (m, 2H), 7.55-7.38 (m, 3H), 6.99 (d, J=8.4 Hz, 1H), 6.83-6.69 (m, 3H), 6.09 (br d, J=8.8 Hz, 1H), 3.87-3.77 (m, 2H), 3.33 (quin, J=5.6 Hz, 1H), 3.27-3.18 (m, 1H), 2.74 (q, J=8.2 Hz, 1H), 2.56 (d, J=2.4 Hz, 1H), 2.28 (d, J=3.6 Hz, 3H), 2.26 (d, J=2.0 Hz, 3H), 1.99-1.86 (m, 2H).

### Example 278: 2-Methyl-5-(2-(methylamino)ethoxy)-N-(3-(naphthalen-1-yl)azetidin-3-yl)benzamide (Compound 453)

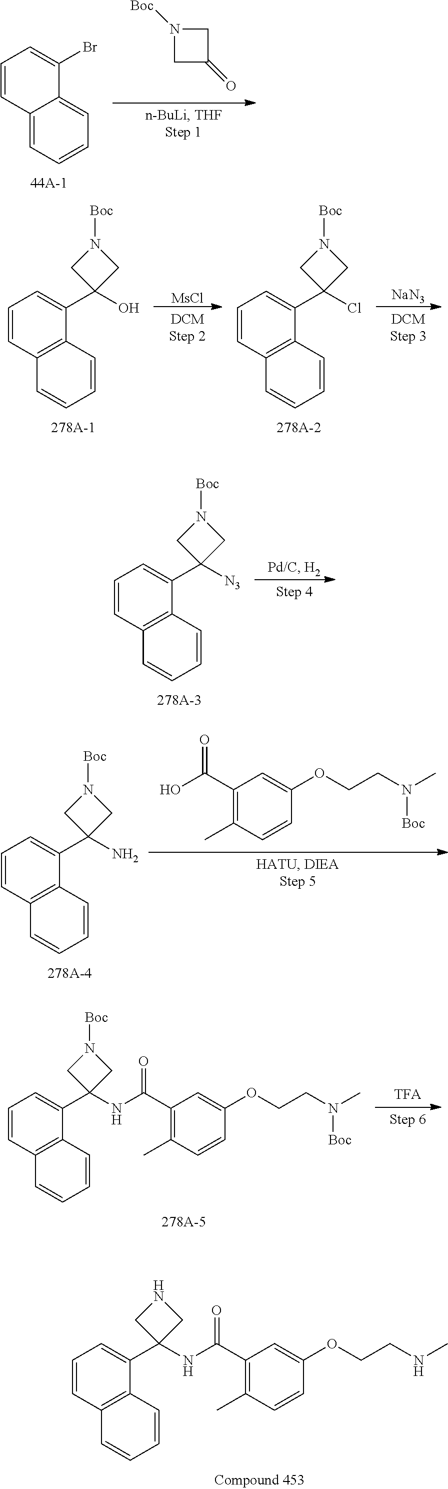

### Step 1: tert-Butyl 3-hydroxy-3-(naphthalen-1-yl)azetidine-1-carboxylate (278A-1)

To a solution of 1-bromonaphthalene (5.00 g, 24.2 mmol, 3.36 mL, 1.0 eq) in THF (30 mL) was added n-BuLi (2.5 M in hexane, 29.0 mL, 3.0 eq) dropwise at −78° C. under a N₂ atmosphere. The mixture was stirred at −78° C. for 1 h. To the mixture was added dropwise the solution of tert-butyl 3-oxoazetidine-1-carboxylate (6.20 g, 36.2 mmol, 1.5 eq) in THF (30 mL) at −78° C. The mixture was stirred at −78° C. for 4 h. LCMS indicated that the starting material was completely consumed, and the desired mass was detected. The mixture was poured into H₂O (40 mL) and extracted with EtOAc (10 mL×3). The combined organic layers were dried over Na₂SO₄ filtered, and concentrated under vacuum to give a residue which was purified by flash silica gel chromatography using a gradient of EtOAc/petroleum ether from 0/1 to 1/5. tert-Butyl 3-hydroxy-3-(naphthalen-1-yl)azetidine-1-carboxylate (3.80 g, 8.89 mmol, 37% yield) was obtained as a yellow oil. M−56+H⁺=244.0 (LCMS); ¹H NMR (400 MHz, DMSO-d₆) δ 8.00-7.86 (m, 3H), 7.63-7.43 (m, 4H), 4.46 (d, J=9.1 Hz, 2H), 4.33-4.15 (m, 2H), 1.37 (s, 9H).

### Step 2: tert-Butyl 3-chloro-3-(naphthalen-1-yl)azetidine-1-carboxylate (278A-2)

To a solution of tert-butyl 3-hydroxy-3-(naphthalen-1-yl)azetidine-1-carboxylate (1.00 g, 3.34 mmol, 1.0 eq) in DCM (10 mL) was added TEA (676 mg, 6.68 mmol, 930 μL, 2.0 eq), followed by MsCl (765 mg, 6.68 mmol, 517 μL, 2.0 eq) dropwise at 0° C. The mixture was stirred at 20° C. for 16 h under a N₂ atmosphere. LCMS indicated that the starting material was completely consumed, and the desired mass was detected. The mixture was poured into H₂O (10 mL) and extracted with EtOAc (30 mL×3). The combined organic layers were dried over Na₂SO₄ filtered, and concentrated under vacuum to give a residue which was purified by flash silica gel chromatography using a gradient of EtOAc/petroleum ether from 0/1 to 1/10. tert-Butyl 3-chloro-3-(naphthalen-1-yl)azetidine-1-carboxylate (520 mg, 1.64 mmol, 50% yield) was obtained as a colourless oil. M−56+H⁺=262.1 (LCMS).

### Step 3: tert-Butyl 3-azido-3-(naphthalen-1-yl)azetidine-1-carboxylate (278A-3)

To a solution of tert-butyl 3-chloro-3-(naphthalen-1-yl)azetidine-1-carboxylate (500 mg, 1.57 mmol, 1.0 eq) in DMF (6.0 mL) was added NaN₃ (205 mg, 3.15 mmol, 2.0 eq) in portions at 0° C. The resulting mixture was stirred at 100° C. for 16 h under a N₂ atmosphere. LCMS indicated that the starting material was completely consumed. The mixture was poured into H₂O (5.0 mL) and extracted with EtOAc (5.0 mL×3). The combined organic layers were dried over Na₂SO₄, filtered, and concentrated under vacuum to give a residue which was purified by flash silica gel chromatography using pure DCM. tert-Butyl 3-azido-3-(naphthalen-1-yl)azetidine-1-carboxylate (400 mg, 1.23 mmol, 78% yield) was obtained as a yellow oil. M−56+H⁺=269.1 (LCMS).

### Step 4: tert-Butyl 3-amino-3-(naphthalen-1-yl)azetidine-1-carboxylate (278A-4)

To a solution of tert-butyl 3-azido-3-(naphthalen-1-yl)azetidine-1-carboxylate (400 mg, 1.23 mmol, 1.0 eq) in TFE (6.0 mL) was added 10% palladium on carbon (40.0 mg) under a N₂ atmosphere. The resulting mixture was stirred at 20° C. under a H₂ (15 psi) atmosphere for 2 h.

LCMS indicated that the starting material was completely consumed, and the desired mass was detected. The suspension was filtered through a pad of Celite and the filtrate was concentrated under vacuum to give the crude product tert-butyl 3-amino-3-(naphthalen-1-yl)azetidine-1-carboxylate (330 mg) as a yellow oil, which was used in the next step without any further purification. M+H⁺=299.2 (LCMS).

### Step 5: tert-Butyl 3-(5-(2-((tert-butoxycarbonyl)(methyl)amino)ethoxy)-2-methyl benzamido)-3-(naphthalen-1-yl)azetidine-1-carboxylate (278A-5)

To a solution of tert-butyl 3-amino-3-(naphthalen-1-yl)azetidine-1-carboxylate (150 mg, 503 μmol, 1.0 eq) and 5-(2-((tert-butoxycarbonyl)(methyl)amino)ethoxy)-2-methylbenzoic acid (171 mg, 553 μmol, 1.1 eq) in DMF (1.0 mL) were added HATU (287 mg, 754 μmol, 1.5 eq) and DIEA (195 mg, 1.51 mmol, 263 μL, 3.0 eq). The mixture was stirred at 20° C. for 5 h. LCMS indicated that the starting material was completely consumed, and the desired mass was detected. The mixture was poured into H₂O (5.0 mL) and extracted with EtOAc (5.0 mL×3). The combined organic layers were dried over Na₂SO₄, filtered, and concentrated under vacuum to give the crude tert-butyl 3-(5-(2-((tert-butoxycarbonyl)(methyl)amino)ethoxy)-2-methylbenzamido)-3-(naphthalen-1-yl)azetidine-1-carboxylate (160 mg, 271 μmol, 54% yield) as a yellow oil. M+H⁺=590.6 (LCMS).

### Step 6: 2-Methyl-5-(2-(methylamino)ethoxy)-N-(3-(naphthalen-1-yl)azetidin-3-yl)benzamide (Compound 453)

To a solution of tert-butyl 3-(5-(2-((tert-butoxycarbonyl)(methyl)amino)ethoxy)-2-methyl benzamido)-3-(naphthalen-1-yl)azetidine-1-carboxylate (160 mg, 244 μmol, 1.0 eq) in DCM (2.0 mL) was added TFA (1.06 g, 9.26 mmol, 686 μL, 38 eq). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Gemini C18 column (80×30 mm, 3 μm); flow rate: 25 mL/min; gradient: 5%-35% B over 8 min; mobile phase A: 0.1% aqueous TFA, mobile phase B: acetonitrile). 2-Methyl-5-(2-(methylamino)ethoxy)-N-(3-(naphthalen-1-yl)azetidin-3-yl)benzamide (86.4 mg, 222 μmol, 91% yield, TFA salt) was obtained as a white solid. M+H⁺=390.1 (LCMS); ¹H NMR (400 MHZ, DMSO-d₆) δ 9.65 (s, 1H), 9.58-9.44 (m, 1H), 9.01-8.84 (m, 1H), 8.63 (br s, 2H), 8.06-8.01 (m, 1H), 7.96 (br d, J=8.0 Hz, 1H), 7.88-7.83 (m, 1H), 7.80 (br d, J=7.1 Hz, 1H), 7.63-7.55 (m, 3H), 7.13 (d, J=8.6 Hz, 1H), 6.94 (dd, J=2.2, 8.3 Hz, 1H), 6.76 (d, J=2.1 Hz, 1H), 4.84 (br s, 2H), 4.79-4.67 (m, 2H), 4.12 (br t, J=4.6 Hz, 2H), 3.29 (br s, 2H), 2.61 (br s, 3H), 2.06 (s, 3H).

### Example 279: 2-Methyl-N-(1-methyl-3-(naphthalen-1-yl)azetidin-3-yl)-5-(2-(methylamino)ethoxy)benzamide (Compound 499)

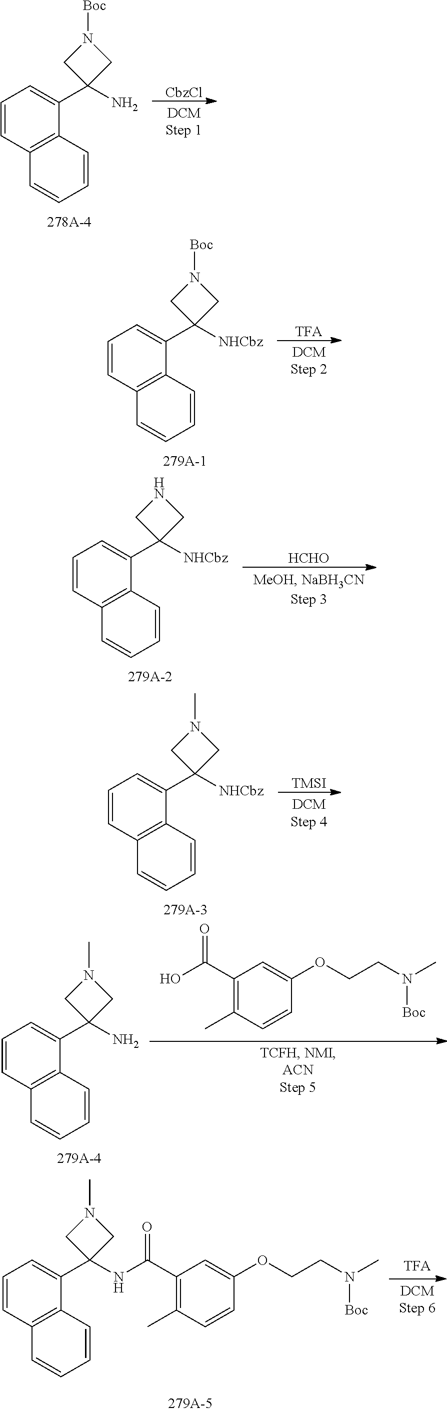

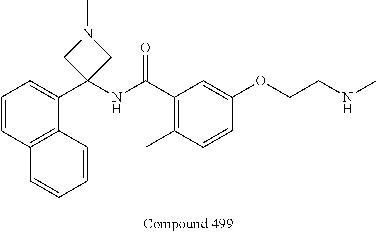 [SMILES: CNCCOc1ccc(C)c(C(=O)NC2(c3cccc4ccccc34)CN(C)C2)c1]

### Step 1: tert-Butyl 3-(((benzyloxy) carbonyl)amino)-3-(naphthalen-1-yl)azetidine-1-carboxylate (279A-1)

To a solution of tert-butyl 3-amino-3-(naphthalen-1-yl)azetidine-1-carboxylate (180 mg, 603 μmol, 1.0 eq) in DCM (1.0 mL) were added DIEA (85.8 mg, 664 μmol, 116 μL, 1.1 eq) and Cb₂Cl (226 mg, 1.33 mmol, 189 UL, 2.2 eq) at 0° C. The mixture was stirred at 20° C. for 2 h.

LCMS indicated that the starting material was completely consumed, and the desired compound was detected. The mixture was poured into H₂O (5.0 mL) and extracted with EtOAc (5.0 mL×3). The combined organic layers were dried over Na₂SO₄, filtered, and concentrated under vacuum to give a residue which was purified by preparative TLC (EtOAc/petroleum ether=1/5, R_{f}=0.5). tert-Butyl 3-(((benzyloxy) carbonyl)amino)-3-(naphthalen-1-yl)azetidine-1-carboxylate (80.0 mg, 185 μmol, 31% yield) was obtained as a yellow oil. M+H⁺=433.3 (LCMS).

### Step 2: Benzyl(3-(naphthalen-1-yl)azetidin-3-yl)carbamate (279A-2)

To a solution of tert-butyl 3-(((benzyloxy) carbonyl)amino)-3-(naphthalen-1-yl)azetidine-1-carboxylate (80.0 mg, 166 μmol, 1.0 eq) in DCM (4.0 mL) was added TFA (1.0 mL). The mixture was stirred at 20° C. for 2 h. LCMS indicated that the starting material was completely consumed, and the desired mass was detected. The mixture was concentrated under vacuum at 20° C. to give the crude product benzyl(3-(naphthalen-1-yl)azetidin-3-yl)carbamate (50.0 mg) as a yellow oil, which was used in the next step without any further purification. M+H⁺=333.1 (LCMS).

### Step 3: Benzyl(1-methyl-3-(naphthalen-1-yl)azetidin-3-yl)carbamate (279A-3)

To a solution of benzyl(3-(naphthalen-1-yl)azetidin-3-yl)carbamate (50.0 mg, 150 μmol, 1.0 eq) in MeOH (1.0 mL) was added formaldehyde (6.77 mg, 226 μmol, 6.22 μL, 37% purity in water, 1.5 eq). The resulting mixture was adjusted to pH 6 with a small amount of AcOH. The mixture was stirred at 20° C. for 30 min, then NaBH₃CN (56.7 mg, 903 μmol, 6.0 eq) was added. The resulting reaction mixture was stirred at 20° C. for another 16 h. LCMS indicated that the starting material was completely consumed, and the desired mass was detected. The mixture was poured into H₂O (5.0 mL) and extracted with EtOAc (5.0 mL×3). The combined organic layers were dried over Na₂SO₄, filtered, and concentrated under vacuum to give a residue which was purified by preparative TLC (EtOAc/petroleum ether=1/10, R_{f}=0.3). Benzyl(1-methyl-3-(naphthalen-1-yl)azetidin-3-yl)carbamate (50.0 mg, 144 μmol, 96% yield) was obtained as a white solid. M+H⁺=347.2 (LCMS).

### Step 4: 1-Methyl-3-(naphthalen-1-yl)azetidin-3-amine (279A-4)

To a solution of benzyl(1-methyl-3-(naphthalen-1-yl)azetidin-3-yl)carbamate (50.0 mg, 130 μmol, 1.0 eq) in DCM (500 μL) was added TMSI (130 mg, 450 μmol, δ8.4 μL, 5.0 eq) in portions at 0° C. The resulting mixture was stirred at 20° C. for 8 h. LCMS indicated that the starting material was consumed, and the desired mass was detected. The mixture was poured into saturated aqueous NH₄Cl (5.0 mL) and extracted with DCM (10 mL×3). But the desired compound was hydrophilic. The aqueous layer was lyophilized to give the crude product 1-methyl-3-(naphthalen-1-yl)azetidin-3-amine (25.0 mg), which was used in the next step without any further purification. M+H⁺=213.1 (LCMS).

### Step 5: tert-Butylmethyl(2-(4-methyl-3-((1-methyl-3-(naphthalen-1-yl)azetidin-3-yl)carbamoyl)phenoxy)ethyl)carbamate (279A-5)

To a solution of 5-(2-((tert-butoxycarbonyl)(methyl)amino)ethoxy)-2-methylbenzoic acid (32.1 mg, 104 μmol, 1.1 eq) in acetonitrile (1.0 mL) were added TCFH (31.7 mg, 113 μmol, 1.2 eq), 1-methylimidazole (23.2 mg, 283 μmol, 22.5 μL, 3.0 eq) and 1-methyl-3-(naphthalen-1-yl)azetidin-3-amine (20.0 mg, 94.2 μmol, 1.0 eq). The resulting mixture was stirred at 20° C. for 3 h. LCMS indicated that the starting material was completely consumed, and the desired mass was detected. The mixture was poured into water (5.0 mL) and extracted with EtOAc (10 mL×3). The combined organic layers were dried over Na₂SO₄, filtered, and concentrated under vacuum to give a residue which was purified by preparative TLC (DCM/MeOH=10/1, R_{f}=0.5). tert-Butylmethyl(2-(4-methyl-3-((1-methyl-3-(naphthalen-1-yl)azetidin-3-yl)carbamoyl) phenoxy)ethyl)carbamate (40.0 mg, 79.4 μmol, 84% yield) was obtained as a yellow oil. M+H⁺=504.3 (LCMS).

### Step 6: 2-Methyl-N-(1-methyl-3-(naphthalen-1-yl)azetidin-3-yl)-5-(2-(methylamino)ethoxy)benzamide (Compound 499)

To a solution of tert-butylmethyl(2-(4-methyl-3-((1-methyl-3-(naphthalen-1-yl)azetidin-3-yl)carbamoyl)phenoxy)ethyl)carbamate (35.0 mg, 62.6 μmol, 1.0 eq) in DCM (2.0 mL) was added TFA (875 μL) at 0° C. The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna C18 column (80×30 mm, 3 μm)); flow rate: 25 mL/min; gradient: 1%-30% B over 8 min; mobile phase A: 0.1% aqueous TFA, mobile phase B: acetonitrile). 2-Methyl-N-(1-methyl-3-(naphthalen-1-yl)azetidin-3-yl)-5-(2-(methylamino)ethoxy)benzamide (7.50 mg, 18.0 μmol, 29% yield, TFA salt) was obtained as a white solid. M+H⁺=404.1 (LCMS), ¹H NMR (400 MHZ, DMSO-d₆) δ 9.40 (br s, 1H), 8.81-8.34 (m, 1H), 8.05-7.99 (m, 1H), 7.96 (d, J=8.2 Hz, 1H), 7.90-7.83 (m, 1H), 7.74 (br s, 1H), 7.60-7.55 (m, 3H), 7.12 (d, J=8.4 Hz, 1H), 6.94 (dd, J=2.8, 8.4 Hz, 1H), 6.80 (d, J=2.7 Hz, 1H), 5.17-4.69 (m, 4H), 4.16 (t, J=5.2 Hz, 2H), 3.29 (t, J=5.2 Hz, 2H), 2.95 (br s, 3H), 2.64 (s, 3H), 2.07 (s, 3H).

### Example 280: 2-Methyl-5-(2-(methylamino)ethoxy)-N-(3-(naphthalen-1-yl)oxetan-3-yl)benzamide (Compound 451)

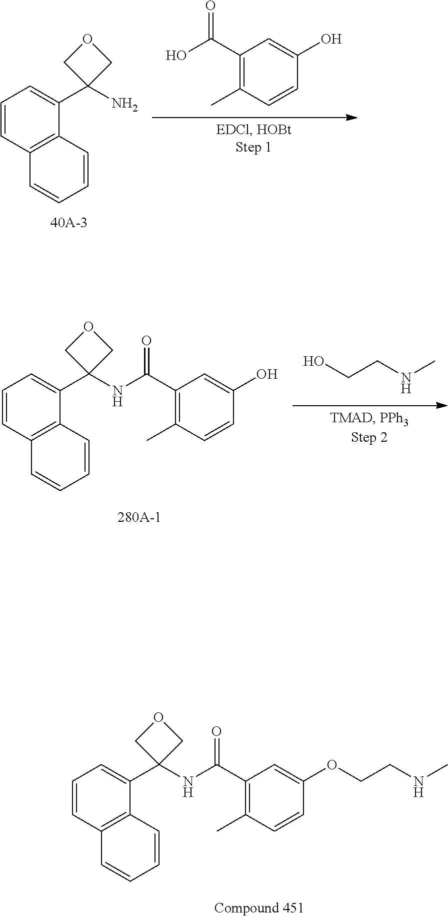

### Step 1: 5-Hydroxy-2-methyl-N-(3-(naphthalen-1-yl)oxetan-3-yl)benzamide (280A-1)

To a solution of 3-(naphthalen-1-yl)oxetan-3-amine (250 mg, 1.25 mmol, 0.80 eq) and 5-hydroxy-2-methylbenzoic acid (239 mg, 1.57 mmol, 1.0 eq) in DMF (8.0 mL) were added TEA (159 mg, 1.57 mmol, 218 μL, 1.0 eq), EDCI (316 mg, 1.65 mmol, 1.0 eq) and HOBt (42.4 mg, 314 μmol, 0.20 eq). The mixture was stirred at 20° C. for 16 h. LCMS indicated that the starting material was completely consumed, and the desired mass was detected. The reaction mixture was poured into H₂O (10 mL) and extracted with DCM (5.0 mL×2). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 25%-55% B over 8 min; mobile phase A: 0.04% aqueous HCl, mobile phase B: acetonitrile). 5-Hydroxy-2-methyl-N-(3-(naphthalen-1-yl)oxetan-3-yl)benz amide (80.0 mg, 216 μmol, 14% yield, HCl salt) was obtained as a white solid. M+H⁺=334.1 (LCMS).

### Step 2: 2-Methyl-5-(2-(methylamino)ethoxy)-N-(3-(naphthalen-1-yl)oxetan-3-yl)benzamide (Compound 451)

To a solution of 5-hydroxy-2-methyl-N-(3-(naphthalen-1-yl)oxetan-3-yl)benzamide (70.0 mg, 210 μmol, 1.0 eq) and 2-(methylamino)ethanol (15.8 mg, 210 μmol, 16.9 μL, 1.0 eq) in toluene (6.0 mL) were added TMAD (108 mg, 630 μmol, 3.0 eq) and PPh₃ (165 mg, 630 μmol, 3.0 eq) under a N₂ atmosphere. The mixture was stirred at 100° C. for 16 h. LCMS indicated that the starting material was completely consumed, and the desired mass was detected. The reaction mixture was allowed to cool to room temperature, poured into H₂O (10 mL) and extracted with DCM (10 mL×5). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 5%-30% B over 8 min; mobile phase A: 0.04% aqueous HCl, mobile phase B: acetonitrile). 2-Methyl-5-(2-(methylamino)ethoxy)-N-(3-(naphthalen-1-yl)oxetan-3-yl)benzamide (6.00 mg, 15.4 μmol, 7% yield, HCl salt) was obtained as a white solid. M+H⁺=391.1 (LCMS); 1H NMR (400 MHz, DMSO-d₆) δ 9.72 (s, 1H), 8.01-7.96 (m, 1H), 7.88 (d, J=8.2 Hz, 1H), 7.78 (br d, J=6.6 Hz, 2H), 7.57-7.49 (m, 3H), 7.05 (d, J=8.4 Hz, 1H), 6.86 (dd, J=2.6, 8.3 Hz, 1H), 6.67 (d, J=2.7 Hz, 1H), 5.28-5.18 (m, 4H), 3.91 (t, J=5.6 Hz, 2H), 2.75 (t, J=5.6 Hz, 2H), 2.29 (s, 3H), 1.93 (s, 3H).

### Example 281: N-(3-(3-Methoxynaphthalen-1-yl)oxetan-3-yl)-2-methyl-5-((1-methylazetidin-2-yl)methoxy)benzamide (Compound 450)

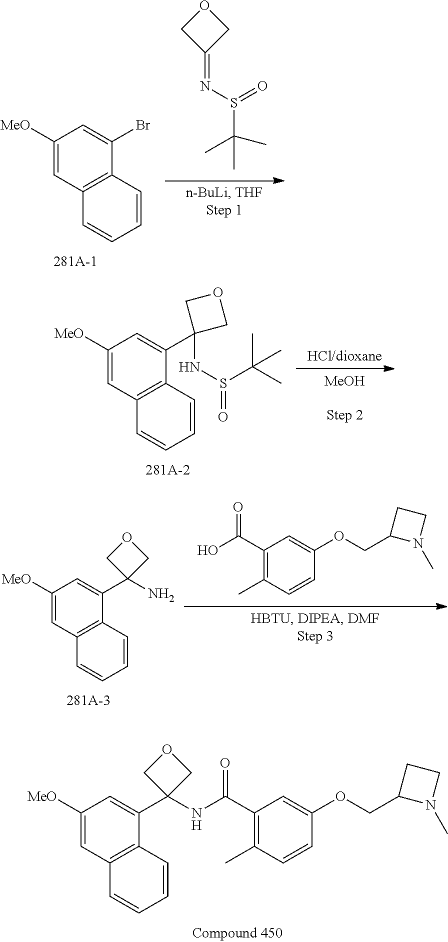 [SMILES: COc1cc(Br)c2ccccc2c1 ; CC(C)(C)S(=O)N=C1COC1 ; COc1cc(C2(NS(=O)C(C)(C)C)COC2)c2ccccc2c1 ; COc1cc(C2(N)COC2)c2ccccc2c1 ; Cc1ccc(OCC2CCN2C)cc1C(=O)O ; COc1cc(C2(NC(=O)c3cc(OCC4CCN4C)ccc3C)COC2)c2ccccc2c1]

### Step 1: N-(3-(3-Methoxynaphthalen-1-yl)oxetan-3-yl)-2-methylpropane-2-sulfinamide (281A-2)

To a solution of 1-bromo-3-methoxynaphthalene (500 mg, 2.11 mmol, 1.0 eq) in THF (5.0 mL) was added n-BuLi (2.5 M in hexane, 1.01 mL, 1.2 eq) dropwise at −78° C. under a N₂ atmosphere, the resulting mixture was stirred at −78° C. for 1 h, then a solution of 2-methyl-N-(oxetan-3-ylidene) propane-2-sulfinamide (554 mg, 3.16 mmol, 1.5 eq) in THF (5.0 mL) was added dropwise at −78° C. The mixture was stirred at −78° C. for 1 h. LCMS indicated that the starting material was completely consumed. The reaction mixture was quenched by addition of saturated aqueous NH₄Cl (10 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/2. N-(3-(3-Methoxynaphthalen-1-yl)oxetan-3-yl)-2-methylpropane-2-sulfinamide (252 mg, 740 μmol, 35% yield) was obtained as a yellow oil. M+H⁺=334.2 (LCMS).

### Step 2:3-(3-Methoxynaphthalen-1-yl)oxetan-3-amine (281A-3)

To a solution of N-(3-(3-methoxynaphthalen-1-yl)oxetan-3-yl)-2-methylpropane-2-sulfinamide (252 mg, 756 μmol, 1.0 eq) in MeOH (3.0 mL) was added HCl/dioxane (4 M, 1.51 mL, 8.0 eq) at 0° C. The mixture was stirred at 0° C. for 30 min. LCMS indicated that the starting material was completely consumed. The reaction mixture was concentrated under vacuum to give a crude product 3-(3-methoxynaphthalen-1-yl)oxetan-3-amine (200 mg, HCl salt) as a yellow solid. M+H⁺=230.2 (LCMS).

### Step 3: N-(3-(3-Methoxynaphthalen-1-yl)oxetan-3-yl)-2-methyl-5-((1-methylazetidin-2-yl)methoxy)benzamide (Compound 450)

To a solution of 3-(3-methoxynaphthalen-1-yl)oxetan-3-amine (40.0 mg, 151 μmol, 1.0 eq, HCl salt) and 2-methyl-5-((1-methylazetidin-2-yl)methoxy)benzoic acid (40.9 mg, 151 μmol, 1.0 eq, HCl salt) in DMF (2.0 mL) were added HBTU (143 mg, 376 μmol, 2.5 eq) and DIEA (97.3 mg, 753 μmol, 131 μL, 5.0 eq). The mixture was stirred at 20° C. for 2 h. LCMS indicated that the starting material was completely consumed. The mixture was concentrated under vacuum to give a residue which was purified by preparative HPLC (Waters Xbridge Prep OBD C18 column (150×40 mm, 10 μm); flow rate: 50 mL/min; gradient: 15%-50% B over 8 min; mobile phase A: 10 mM aqueous NH₄HCO₃, mobile phase B: acetonitrile). N-(3-(3-Methoxynaphthalen-1-yl)oxetan-3-yl)-2-methyl-5-((1-methylazetidin-2-yl)methoxy)benzamide (5.60 mg, 12.5 μmol, 8% yield) was obtained as an off-white gum. M+H⁺==447.3 (LCMS); ¹H NMR (400 MHZ, CD₃OD) δ 7.85 (d, J=8.3 Hz, 1H), 7.55 (d, J=8.4 Hz, 1H), 7.48-7.41 (m, 2H), 7.38-7.30 (m, 1H), 7.27-7.21 (m, 1H), 7.04 (d, J=8.4 Hz, 1H), 6.89-6.80 (m, 1H), 6.69 (d, J=2.6 Hz, 1H), 5.57-5.11 (m, 4H), 3.98-3.85 (m, 5H), 3.54-3.35 (m, 2H), 2.99-2.89 (m, 1H), 2.47-2.32 (m, 3H), 2.15-1.91 (m, 5H).

### Example 282: 5-(2-Acetamidoethoxy)-2-methyl-N-(1-(naphthalen-1-yl)cyclopropyl)benzamide (Compound 425)

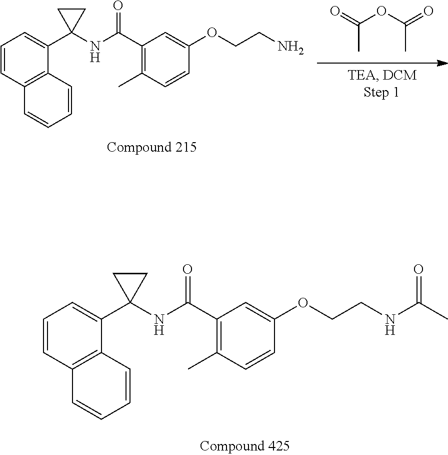

### Step 1: 5-(2-Acetamidoethoxy)-2-methyl-N-(1-(naphthalen-1-yl)cyclopropyl)benzamide (Compound 425)

To a solution of 5-(2-aminoethoxy)-2-methyl-N-(1-(naphthalen-1-yl)cyclopropyl)benzamide (100 mg, 277 μmol, 1.0 eq) in DCM (5.0 mL) were added TEA (112 mg, 1.11 mmol, 154 μL, 4.0 eq) and acetic anhydride (56.6 mg, 555 μmol, 52.0 μL, 2.0 eq). The mixture was stirred at 20° C. for 3 h. LCMS indicated that the starting material was completely consumed, and the desired mass was detected. The reaction mixture was poured into H₂O (4.0 mL) and extracted with DCM (5.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 40%-60% B over 8 min; mobile phase A: 0.04% aqueous HCl, mobile phase B: acetonitrile). (5-(2-Acetamidoethoxy)-2-methyl-N-(1-(naphthalen-1-yl)cyclopropyl)benzamide (21.2 mg, 52.4 μmol, 19% yield) was obtained as a white solid. M+H⁺=403.1 (LCMS); ¹H NMR (400 MHZ, DMSO-d₆) δ 9.10 (s, 1H), 8.66 (br d, J=8.3 Hz, 1H), 8.06 (br s, 1H), 7.93 (br d, J=7.6 Hz, 1H), 7.86-7.77 (m, 2H), 7.61-7.41 (m, 3H), 7.03 (br d, J=7.9 Hz, 1H), 6.99-6.99 (m, 1H), 6.83 (br d, J=7.8 Hz, 1H), 6.62 (br s, 1H), 3.87 (br s, 2H), 3.34 (br d, J=5.0 Hz, 2H), 1.96 (s, 3H), 1.80 (s, 3H), 1.35 (br s, 2H), 1.23-1.07 (m, 2H).

### Example 283: 2-Methyl-5-(2-(methylsulfonamido) ethoxy)-N-(1-(naphthalen-1-yl)cyclopropyl)benzamide (Compound 424)

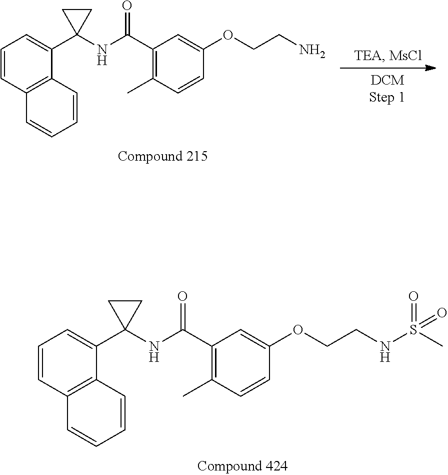 [SMILES: Cc1ccc(OCCN)cc1C(=O)NC1(c2cccc3ccccc23)CC1 ; Cc1ccc(OCCNS(C)(=O)=O)cc1C(=O)NC1(c2cccc3ccccc23)CC1]

### Step 1: 2-Methyl-5-(2-(methylsulfonamido) ethoxy)-N-(1-(naphthalen-1-yl)cyclopropyl)benzamide (Compound 424)

To a solution of 5-(2-aminoethoxy)-2-methyl-N-(1-(naphthalen-1-yl)cyclopropyl)benzamide (100 mg, 277 μmol, 1.0 eq) in DCM (4.0 mL) were added TEA (28.1 mg, 277 μmol, 38.6 μL, 1.0 eq) and MsCl (25.4 mg, 222 μmol, 17.2 μL, 0.8 eq) at 0° C. The mixture was stirred at 20° C. for 3 h. LCMS indicated that the starting material was completely consumed, and the desired mass was detected. The reaction mixture was poured into H₂O (4.0 mL) and extracted with EtOAc (2.0 mL×3). The combined organic layers were dried over Na₂SO₄ filtered, and concentrated under vacuum to give a residue which was purified by preparative HPLC (Phenomenex Luna C18 column (80×30 mm, 3 μm); flow rate: 25 mL/min; gradient: 45%-60% B over 8 min; mobile phase A: 0.04% aqueous HCl, mobile phase B: acetonitrile). 2-Methyl-5-(2-(methylsulfonamido) ethoxy)-N-(1-(naphthalen-1-yl)cyclopropyl)benzamide (37.2 mg, 84.8 μmol, 30% yield, HCl salt) was obtained as a white solid. M+H⁺=439.1 (LCMS); ¹H NMR (400 MHZ, DMSO-d₆) δ 9.11 (s, 1H), 8.65 (d, J=8.3 Hz, 1H), 7.93 (d, J=7.9 Hz, 1H), 7.81 (t, J=7.8 Hz, 2H), 7.62-7.42 (m, 3H), 7.25 (t, J=5.9 Hz, 1H), 7.04 (d, J=8.4 Hz, 1H), 6.84 (dd, J=2.7, 8.3 Hz, 1H), 6.62 (d, J=2.6 Hz, 1H), 3.93 (t, J=5.5 Hz, 2H), 3.29-3.23 (m, 2H), 2.91 (s, 3H), 1.95 (s, 3H), 1.35 (s, 2H), 1.20-1.13 (m, 2H).

### Example 284: 5-(((2R,4S)-4-Hydroxy-1-methylpyrrolidin-2-yl)methoxy)-2-methyl-N-(1-(naphthalen-1-yl)cyclopropyl)benzamide (Compound 439)

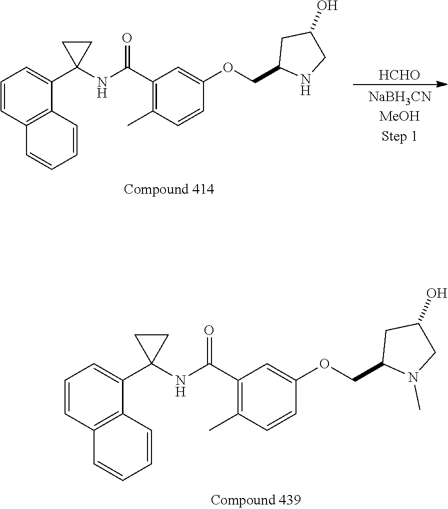

### Step 1: 5-(((2R,4S)-4-Hydroxy-1-methylpyrrolidin-2-yl)methoxy)-2-methyl-N-(1-(naphthalen-1-yl)cyclopropyl)benzamide (Compound 439)

To a solution of (5-(((2R,4S)-4-hydroxypyrrolidin-2-yl)methoxy)-2-methyl-N-(1-(naphthalen-1-yl)cyclopropyl)benzamide (60.0 mg, 144 μmol, 1.0 eq) in MeOH (6.0 mL) was added TEA (20 μL), followed by the addition of formaldehyde (16.1 μL, 216 μmol, 37% purity in water, 1.5 eq). The resulting mixture was adjusted to pH 6 with a small amount of AcOH. The mixture was stirred at 20° C. for 30 min, then NaBH₃CN (54.3 mg, 864 μmol, 6.0 eq) was added. The resulting reaction mixture was stirred at 20° C. for another 16 h. LCMS indicated that the starting material was completely consumed. The mixture was treated with H₂O (10 mL) and extracted with EtOAc (10 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 5%-50% B over 8 min; mobile phase A: 0.04% aqueous HCl, mobile phase B: acetonitrile). 5-(((2R,4S)-4-Hydroxy-1-methylpyrrolidin-2-yl)methoxy)-2-methyl-N-(1-(naphthalen-1-yl)cyclopropyl)benzamide (35.6 mg, 81.6 μmol, 57% yield) was obtained as a white solid. M+H⁺=431.2 (LCMS); ¹H NMR (400 MHz, DMSO-d₆) δ 10.21 (br d, J=5.1 Hz, 1H), 9.10 (s, 1H), 8.65 (d, J=8.4 Hz, 1H), 7.93 (d, J=7.4 Hz, 1H), 7.83 (t, J=6.6 Hz, 2H), 7.59-7.44 (m, 3H), 7.08 (d, J=8.6 Hz, 1H), 6.90 (dd, J=2.8, 8.4 Hz, 1H), 6.69 (d, J=2.6 Hz, 1H), 5.66-5.48 (m, 1H), 4.46-4.34 (m, 1H), 4.32-4.23 (m, 1H), 4.18 (dd, J=7.6, 10.9 Hz, 1H), 4.00-3.88 (m, 1H), 3.69 (td, J=5.9, 11.5 Hz, 1H), 3.08-3.00 (m, 1H), 2.96 (d, J=4.8 Hz, 3H), 2.14-2.07 (m, 1H), 1.99-1.91 (m, 4H), 1.36 (br s, 2H), 1.20-1.15 (m, 2H).

### Example 285: 5-(((2R,4R)-4-Hydroxy-1-methylpyrrolidin-2-yl)methoxy)-2-methyl-N-(1-(naphthalen-1-yl)cyclopropyl)benzamide (Compound 440)

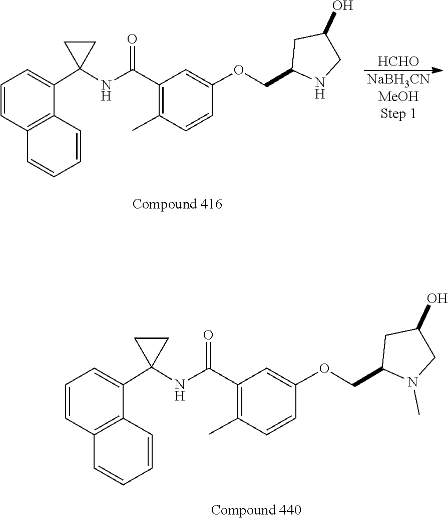

### Step 1: 5-(((2R,4R)-4-Hydroxy-1-methylpyrrolidin-2-yl)methoxy)-2-methyl-N-(1-(naphthalen-1-yl)cyclopropyl)benzamide (Compound 440)

To a solution of 5-(((2R,4R)-4-hydroxypyrrolidin-2-yl)methoxy)-2-methyl-N-(1-(naphthalen-1-yl)cyclopropyl)benzamide (150 mg, 360 μmol, 1.0 eq) in MeOH (2.0 mL) was added TEA (50 μL), followed by the addition of formaldehyde (40.2 μL, 540 μmol, 37% purity in water, 1.5 eq). The resulting mixture was adjusted to pH 6 with a small amount of AcOH. The mixture was stirred at 20° C. for 30 min, then NaBH₃CN (136 mg, 2.16 mmol, 6.0 eq) was added. The resulting reaction mixture was stirred at 20° C. for another 16 h. LCMS indicated that the starting material was completely consumed. The mixture was treated with water (10 mL) and extracted with EtOAc (10 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 5%-50% B over 8 min; mobile phase A: 0.04% aqueous HCl, mobile phase B: acetonitrile). 5-(((2R,4R)-4-Hydroxy-1-methylpyrrolidin-2-yl)methoxy)-2-methyl-N-(1-(naphthalen-1-yl)cyclopropyl)benzamide (86.5 mg, 201 μmol, 56% yield, HCl salt) was obtained as a white solid. M+H⁺=431.2 (LCMS); ¹H NMR (400 MHZ, DMSO-d₆) δ 10.10 (br d, J=5.1 Hz, 1H), 9.10 (s, 1H), 8.66 (br d, J=7.8 Hz, 1H), 7.93 (br d, J=7.8 Hz, 1H), 7.83 (br t, J=6.9 Hz, 2H), 7.62-7.42 (m, 3H), 7.08 (br d, J=8.1 Hz, 1H), 6.95-6.85 (m, 1H), 6.69 (br s, 1H), 5.55 (br s, 1H), 4.41 (br s, 1H), 4.30-4.21 (m, 1H), 4.20-4.11 (m, 1H), 3.89-3.75 (m, 1H), 3.46-3.36 (m, 2H), 3.21-3.16 (m, 1H), 2.91 (br d, J=2.3 Hz, 3H), 1.96 (s, 3H), 1.73-1.62 (m, 1H), 1.36 (br s, 2H), 1.19 (br s, 2H).

### Example 286: 5-(((2R,4R)-4-Fluoropyrrolidin-2-yl)methoxy)-2-methyl-N-(1-(naphthalen-1-yl)cyclopropyl)benzamide (Compound 438)

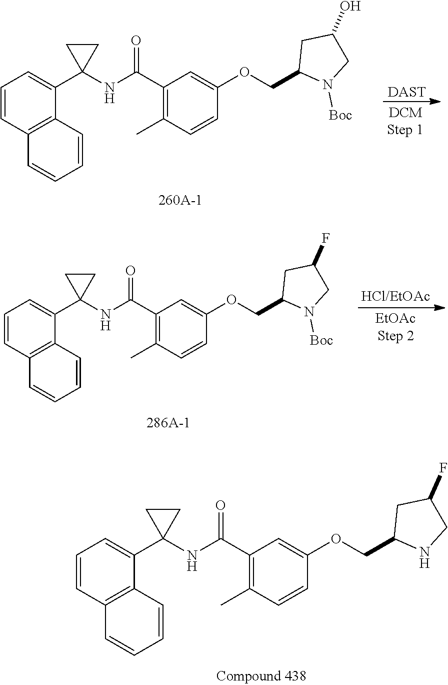

### Step 1: (2R,4R)-tert-Butyl 4-fluoro-2-((4-methyl-3-((1-(naphthalen-1-yl)cyclopropyl)carbamoyl)phenoxy)methyl)pyrrolidine-1-carboxylate (286A-1)

A solution of (2R,4S)-tert-butyl 4-hydroxy-2-((4-methyl-3-((1-(naphthalen-1-yl)cyclopropyl)carbamoyl)phenoxy)methyl)pyrrolidine-1-carboxylate (130 mg, 352 μmol, 1.0 eq) in DCM (2.0 mL) was degassed and purged with N₂ three times. To this solution was added DAST (101 mg, 629 μmol, 83.1 μL, 2.5 eq) dropwise at −78° C. The mixture was warmed to room temperature and stirred under a N₂ atmosphere for 16 h. LCMS indicated that the starting material was completely consumed. The mixture was poured into ice water (10 mL) and extracted with EtOAc (10 mL×3). The combined organic layers were dried over Na₂SO₄, filtered, and concentrated under vacuum to give the crude (2R,4R)-tert-butyl 4-fluoro-2-((4-methyl-3-((1-(naphthalen-1-yl)cyclopropyl)carbamoyl)phenoxy)methyl)pyrrolidine-1-carboxylate (120 mg, 231 μmol, 92% yield) as a white solid, which was used in the next step without any further purification. M+H⁺=519.3 (LCMS).

### Step 2: 5-(((2R,4R)-4-fluoropyrrolidin-2-yl)methoxy)-2-methyl-N-(1-(naphthalen-1-yl)cyclopropyl)benzamide (Compound 438)

To a solution of (2R,4R)-tert-butyl 4-fluoro-2-((4-methyl-3-((1-(naphthalen-1-yl)cyclopropyl) carbamoyl)phenoxy)methyl)pyrrolidine-1-carboxylate (120 mg, 208 μmol, 1.0 eq) in EtOAc (1.0 mL) was added HCl/EtOAc (4 M, 3.6 mL). The mixture was stirred at 20° C. for 2 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 1%-35% B over 8 min; mobile phase A: 0.04% aqueous HCl, mobile phase B: acetonitrile). 5-(((2R,4R)-4-Fluoropyrrolidin-2-yl)methoxy)-2-methyl-N-(1-(naphthalen-1-yl)cyclopropyl)benzamide (16.2 mg, 37.0 μmol, 18% yield, HCl salt) was obtained as a white solid. M+H⁺=419.1 (LCMS); ¹H NMR (400 MHZ, DMSO-d₆) δ 10.08-9.85 (m, 1H), 9.47-9.25 (m, 1H), 9.12 (s, 1H), 8.66 (d, J=8.4 Hz, 1H), 7.93 (d, J=7.6 Hz, 1H), 7.83 (t, J=6.6 Hz, 2H), 7.61-7.43 (m, 3H), 7.08 (d, J=8.5 Hz, 1H), 6.89 (dd, J=2.8, 8.3 Hz, 1H), 6.67 (d, J=2.6 Hz, 1H), 5.58-5.31 (m, 1H), 4.25-4.13 (m, 1H), 4.08-3.93 (m, 2H), 3.63-3.47 (m, 1H), 3.47-3.35 (m, 1H), 2.63-2.54 (m, 1H), 2.10-1.99 (m, 1H), 1.97 (s, 3H), 1.36 (s, 2H), 1.22-1.13 (m, 2H).

### Example 287: 5-(((2R,4R)-4-Fluoro-1-methylpyrrolidin-2-yl)methoxy)-2-methyl-N-(1-(naphthalen-1-yl)cyclopropyl)benzamide (Compound 437)

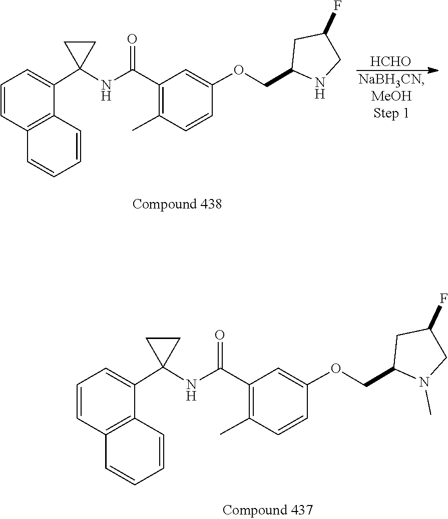

### Step 1: 5-(((2R,4R)-4-Fluoro-1-methylpyrrolidin-2-yl)methoxy)-2-methyl-N-(1-(naphthalene-1-yl)cyclopropyl)benzamide (Compound 437)

To a solution of 5-(((2R,4R)-4-fluoropyrrolidin-2-yl)methoxy)-2-methyl-N-(1-(naphthalen-1-yl)cyclopropyl)benzamide (40.0 mg, 95.6 μmol, 1.0 eq) in MeOH (6.0 mL) was added TEA (13 μL), followed by the addition of formaldehyde (10.7 μL, 143 μmol, 37% purity in water, 1.5 eq). The resulting mixture was adjusted to pH 6 with a small amount of AcOH. The mixture was stirred at 20° C. for 30 min, then NaBH₃CN (36.0 mg, 573 μmol, 6.0 eq) was added. The resulting reaction mixture was stirred at 20° C. for another 16 h. LCMS indicated that the starting material was completely consumed. The mixture was treated with water (10 mL) and extracted with EtOAc (10 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 5%-40% B over 8 min; mobile phase A: 0.04% aqueous HCl, mobile phase B: acetonitrile). 5-(((2R,4R)-4-Fluoro-1-methylpyrrolidin-2-yl)methoxy)-2-methyl-N-(1-(naphthalene-1-yl)cyclopropyl)benzamide (15.3 mg, 34.1 μmol, 36% yield) was obtained as a white solid. M+H⁺=433.1 (LCMS); ¹H NMR (400 MHZ, DMSO-d₆) δ 10.55-10.26 (m, 1H), 9.10 (s, 1H), 8.65 (d, J=8.3 Hz, 1H), 7.93 (d, J=7.8 Hz, 1H), 7.87-7.79 (m, 2H), 7.60-7.43 (m, 3H), 7.09 (d, J=8.5 Hz, 1H), 6.91 (dd, J=2.6, 8.4 Hz, 1H), 6.70 (br d, J=2.4 Hz, 1H), 5.59-5.30 (m, 1H), 4.36-4.23 (m, 1H), 4.15 (br d, J=9.8 Hz, 1H), 3.99-3.75 (m, 2H), 3.55-3.39 (m, 1H), 2.96 (br s, 3H), 2.82-2.72 (m, 1H), 2.14-1.98 (m, 1H), 1.96 (s, 3H), 1.36 (s, 2H), 1.18 (br s, 2H).

### Example 288: 5-(((2R,4S)-4-Fluoropyrrolidin-2-yl)methoxy)-2-methyl-N-(1-(naphthalen-1-yl)cyclopropyl)benzamide (Compound 442)

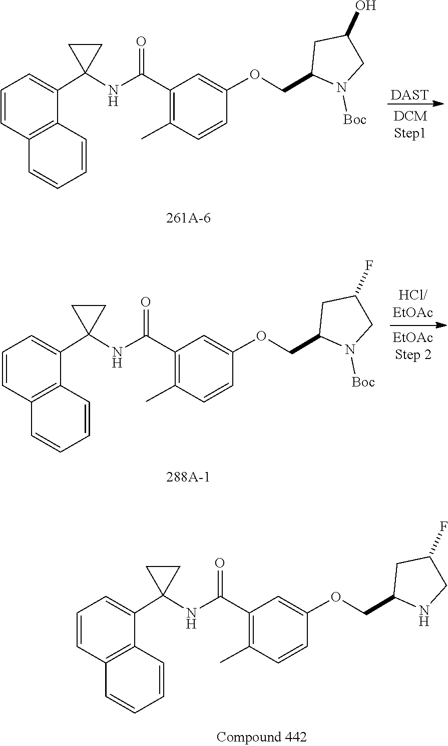

### Step 1: (2R,4S)-tert-Butyl4-fluoro-2-((4-methyl-3-((1-(naphthalen-1-yl)cyclopropyl)carbamoyl)phenoxy)methyl)pyrrolidine-1-carboxylate (288A-1)

A solution of (2R,4R)-tert-butyl 4-hydroxy-2-((4-methyl-3-((1-(naphthalen-1-yl)cyclopropyl) carbamoyl)phenoxy)methyl)pyrrolidine-1-carboxylate (400 mg, 774 μmol, 1.0 eq) in DCM (10 mL) was degassed and purged with N₂ three times. To this solution was added DAST (312 mg, 1.94 mmol, 256 μL, 2.5 eq) dropwise at −78° C. The mixture was warmed to 20° C. and stirred another 16 h under a N₂ atmosphere. LCMS indicated that the starting material was completely consumed, and the desired mass was detected. The reaction mixture was poured into H₂O (10 mL) and extracted with DCM (10 mL×3). The combined organic layers were dried over Na₂SO₄, filtered, and concentrated under vacuum to give the crude (2R,4S)-tert-butyl 4-fluoro-2-((4-methyl-3-((1-(naphthalen-1-yl)cyclopropyl)carbamoyl)phenoxy)methyl)pyrrolidine-1-carboxylate (350 mg) as a white solid, which was used in the next step without any further purification. M+H⁺=519.2 (LCMS).

### Step 2: 5-(((2R,4S)-4-Fluoropyrrolidin-2-yl)methoxy)-2-methyl-N-(1-(naphthalen-1-yl)cyclopropyl)benzamide (Compound 442)

To a solution of (2R,4S)-tert-butyl 4-fluoro-2-((4-methyl-3-((1-(naphthalen-1-yl)cyclopropyl) carbamoyl)phenoxy)methyl)pyrrolidine-1-carboxylate (350 mg, 675 μmol, 1.0 eq) in EtOAc (7.0 mL) was added HCl/EtOAc (4 M, 14 mL). The mixture was stirred at 20° C. for 1 h. LCMS indicated that the starting material was completely consumed, and the desired mass was detected. The reaction mixture was concentrated under vacuum to give a residue which was purified by preparative HPLC (Phenomenex Luna column (80×30 mm, 3 μm); flow rate: 25 mL/min; gradient: 5%-35% B over 8 min; mobile phase A: 0.04% aqueous HCl, mobile phase B: acetonitrile). 5-(((2R,4S)-4-Fluoropyrrolidin-2-yl)methoxy)-2-methyl-N-(1-(naphthalen-1-yl)cyclopropyl)benzamide (300 mg, 659 μmol, 98% yield, HCl salt) was obtained as a white solid. M+H⁺=419.2 (LCMS); ¹H NMR (400 MHZ, DMSO-d₆) δ 9.98 (br d, J=2.2 Hz, 1H), 9.68-9.40 (m, 1H), 9.18-9.12 (m, 1H), 8.66 (d, J=8.4 Hz, 1H), 7.93 (d, J=7.9 Hz, 1H), 7.87-7.78 (m, 2H), 7.62-7.44 (m, 3H), 7.11-7.05 (m, 1H), 6.92-6.86 (m, 1H), 6.67 (d, J=2.6 Hz, 1H), 5.65-5.30 (m, 1H), 4.28-4.00 (m, 3H), 3.47 (br d, J=8.6 Hz, 2H), 2.47-2.18 (m, 2H), 1.97 (s, 3H), 1.36 (br s, 2H), 1.23-1.13 (m, 2H).

### Example 289: 5-(((2R,4S)-4-Fluoro-1-methylpyrrolidin-2-yl)methoxy)-2-methyl-N-(1-(naphthalen-1-yl)cyclopropyl)benzamide (Compound 441)

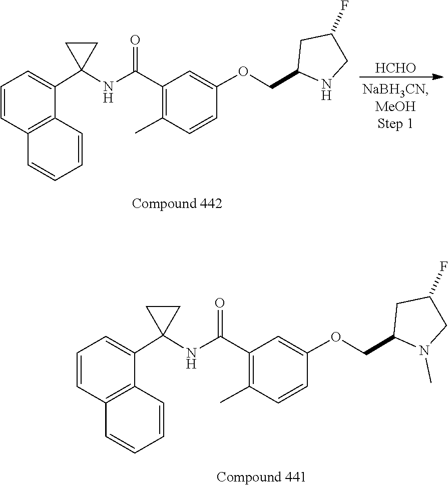

### Step 1: 5-(((2R,4S)-4-Fluoro-1-methylpyrrolidin-2-yl)methoxy)-2-methyl-N-(1-(naphthalen-1-yl)cyclopropyl)benzamide (Compound 441)

To a solution of 5-(((2R,4S)-4-fluoropyrrolidin-2-yl)methoxy)-2-methyl-N-(1-(naphthalen-1-yl)cyclopropyl)benzamide (240 mg, 573 μmol, 1.0 eq) in MeOH (9.0 mL) was added TEA (80.0 μL), followed by the addition of HCHO (93.1 mg, 1.15 mmol, 85.4 μL, 37% purity in water, 2.0 eq). The resulting mixture was treated with a small amount of AcOH (34.4 mg, 573 μmol, 32.8 μL, 1.0 eq) to adjust the pH to 6. The mixture was stirred at 20° C. for 30 min, then NaBH₃CN (72.1 mg, 1.15 mmol, 2.0 eq) was added. The reaction mixture was stirred at 20° C. for another 16 h. LCMS indicated that the starting material was completely consumed, and the desired mass was detected. The mixture was treated with H₂O (10 mL) and extracted with DCM (10 mL×5). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 15%-40% B over 8 min; mobile phase A: 0.04% aqueous HCl, mobile phase B: acetonitrile). 5-(((2R,4S)-4-Fluoro-1-methylpyrrolidin-2-yl)methoxy)-2-methyl-N-(1-(naphthaalen-1-yl)cyclopropyl)benzamide (72.7 mg, 144 μmol, 25% yield, HCl salt) was obtained as a white solid. M+H⁺=433.1 (LCMS); ¹H NMR (400 MHz, DMSO-d₆) δ 11.43-11.24 (m, 1H), 9.14 (s, 1H), 8.66 (br d, J=8.3 Hz, 1H), 7.93 (d, J=8.0 Hz, 1H), 7.86-7.77 (m, 2H), 7.63-7.41 (m, 3H), 7.08 (d, J=8.5 Hz, 1H), 6.96-6.83 (m, 1H), 6.77-6.66 (m, 1H), 5.61-5.26 (m, 1H), 4.47-4.18 (m, 3H), 4.08-3.97 (m, 1H), 3.56-3.35 (m, 1H), 2.96 (br d, J=4.8 Hz, 3H), 2.78-2.65 (m, 1H), 2.22-2.03 (m, 1H), 1.95 (s, 3H), 1.37 (br s, 2H), 1.18 (br s, 2H).

### Example 290: 5-((1-Benzyl-4,4-dimethylazetidin-2-yl)methoxy)-2-methyl-N-(1-(naphthalen-1-yl)cyclopropyl)benzamide (Compound 492) and 5-((1-benzyl-5,5-dimethylpyrrolidin-3-yl)oxy)-2-methyl-N-(1-(naphthalen-1-yl)cyclopropyl)benzamide (Compound 491)

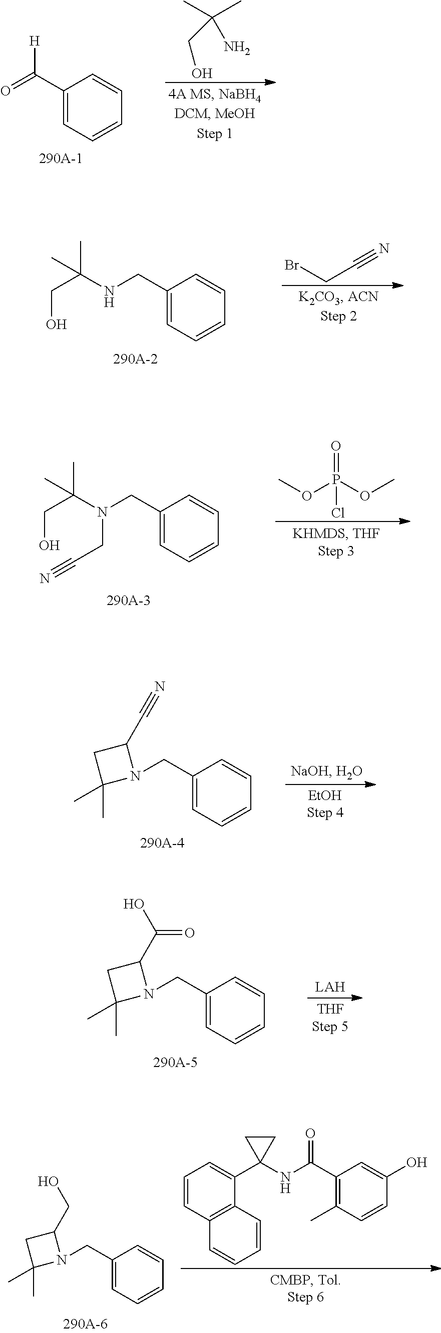

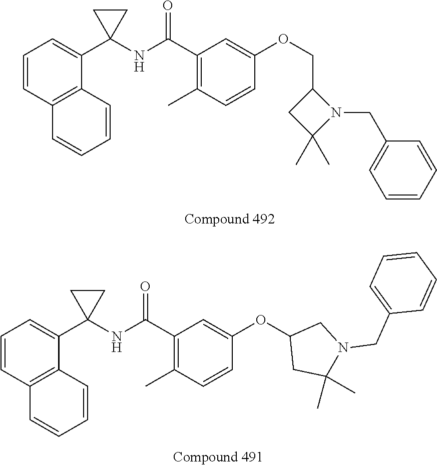 [SMILES: Cc1ccc(OCC2CC(C)(C)N2Cc2ccccc2)cc1C(=O)NC1(c2cccc3ccccc23)CC1 ; Cc1ccc(OC2CN(Cc3ccccc3)C(C)(C)C2)cc1C(=O)NC1(c2cccc3ccccc23)CC1]

### Step 1: 2-(Benzylamino)-2-methylpropan-1-ol (290A-2)

To a solution of benzaldehyde (11.9 g, 112 mmol, 11.3 mL, 1.0 eq) and 2-amino-2-methylpropan-1-ol (10.0 g, 112 mmol, 10.7 mL, 1.0 eq) in DCM (100 mL) was added 4 Å molecular sieve (15.0 g). The mixture was stirred at 20° C. for 16 h. Then the mixture was filtered through a pad of cotton and concentrated under vacuum to give a residue. To this residue was added MeOH (50 mL), followed by NaBH₄ (5.09 g, 135 mmol, 1.2 eq) at 0° C. After the addition, the mixture was stirred at 20° C. for 1 h. LCMS indicated that the starting material was completely consumed, and the desired mass was detected. The reaction mixture was poured into saturated aqueous NH₄Cl (50 mL) and extracted with EtOAc (30 mL×3). The combined organic layers were dried over Na₂SO₄, filtered, and concentrated under vacuum to give a residue which was purified by flash silica gel chromatography using a gradient of EtOAc/petroleum ether from 0/1 to 1/0. 2-(Benzylamino)-2-methylpropan-1-ol (3.00 g, 16.7 mmol, 15% yield) was obtained as a white solid. M+H⁺=180.1 (LCMS); ¹H NMR (400 MHZ, CDCl₃) δ 7.39-7.22 (m, 5H), 3.68 (s, 2H), 3.34 (s, 2H), 1.15 (s, 6H).

### Step 2: 2-(Benzyl(1-hydroxy-2-methylpropan-2-yl)amino) acetonitrile (290A-3)

To a solution of 2-(benzylamino)-2-methylpropan-1-ol (3.00 g, 16.7 mmol, 1.0 eq) in acetonitrile (50 mL) were added 2-bromoacetonitrile (5.62 g, 46.9 mmol, 3.12 mL, 2.8 eq) and K₂CO₃ (3.47 g, 25.1 mmol, 1.5 eq). The mixture was stirred at 100° C. for 16 h. LCMS indicated that the starting material was completely consumed. The reaction mixture was allowed to cool to room temperature, poured into H₂O (40 mL) and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 0/1 to 1/0. 2-(Benzyl(1-hydroxy-2-methylpropan-2-yl)amino) acetonitrile (3.50 g, 16.0 mmol, 96% yield) was obtained as a white solid. ¹H NMR (400 MHZ, DMSO-d₆) δ 7.44-7.16 (m, 5H), 4.75 (t, J=5.2 Hz, 1H), 3.81 (s, 2H), 3.52 (s, 2H), 3.42 (d, J=5.1 Hz, 2H), 1.14 (s, 6H).

### Step 3: 1-Benzyl-4,4-dimethylazetidine-2-carbonitrile (290A-4)

To a solution of 2-(benzyl(1-hydroxy-2-methylpropan-2-yl)amino) acetonitrile (1.70 g, 7.79 mmol, 1.0 eq) in THF (17 mL) was added dimethyl phosphorochloridate (2.25 g, 15.6 mmol, 1.68 mL, 2.0 eq) at −20° C. under a N₂ atmosphere, then KHMDS (1 M in THF, 19.5 mL, 2.5 eq) was added dropwise keeping the temperature below −15° C. The mixture was stirred at −20° C. for 1 h under a N₂ atmosphere. LCMS indicated that the starting material was completely consumed, and the desired mass was detected. The reaction mixture was poured into H₂O (20 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. 1-Benzyl-4,4-dimethylazetidine-2-carbonitrile (640 mg, 3.20 mmol, 41% yield) was obtained as a colorless oil. M+H⁺=201.2 (LCMS); ¹H NMR (400 MHZ, DMSO-d₆) δ 7.39-7.20 (m, 5H), 4.13 (dd, J=6.9, 8.1 Hz, 1H), 3.73-3.65 (m, 1H), 3.61-3.53 (m, 1H), 2.26-2.19 (m, 1H), 2.16-2.09 (m, 1H), 1.20 (d, J=8.3 Hz, 6H).

### Step 4: 1-Benzyl-4,4-dimethylazetidine-2-carboxylic acid (290A-5)

To a solution of 1-benzyl-4,4-dimethylazetidine-2-carbonitrile (300 mg, 1.50 mmol, 1.0 eq) in EtOH (1.5 mL) and H₂O (0.8 mL) was added NaOH (120 mg, 3.00 mmol, 2.0 eq) at room temperature. The mixture was stirred at 80° C. for 16 h. LCMS indicated that the starting material was completely consumed, and the desired mass was detected. The reaction mixture was allowed to cool to room temperature, poured into H₂O (3.0 mL) and extracted with MTBE (3.0 mL×3). The aqueous layer was acidified to pH 5 with HCl (1 M aqueous). The resulting mixture was concentrated under vacuum to remove the water completely. The resulting mixture was treated with MeOH/DCM (V/V=10/1, 10 mL) then filtered. The filter cake was washed with MeOH/DCM (3.0 mL×2) to ensure all product was washed from the solids. The combined organic layers were concentrated under vacuum to give 1-benzyl-4,4-dimethylazetidine-2-carboxylic acid (200 mg, 912 μmol, 61% yield) as a white solid. M+H⁺=220.2 (LCMS); ¹H NMR (400 MHZ, DMSO-d₆) δ 7.40 (d, J=7.1 Hz, 2H), 7.32-7.17 (m, 3H), 3.86-3.70 (m, 3H), 2.06 (t, J=9.6 Hz, 1H), 2.01-1.88 (m, 1H), 1.28 (s, 3H), 1.05 (s, 3H).

### Step 5: (1-Benzyl-4,4-dimethylazetidin-2-yl)methanol (290A-6)

To a solution of 1-benzyl-4,4-dimethylazetidine-2-carboxylic acid (150 mg, 684 μmol, 1.0 eq) in THF (3.0 mL) was added LiAlH₄ (1 M in THF, 1.37 mL, 2.0 eq) dropwise at 0° C. under a N₂ atmosphere. The reaction mixture was stirred at 0° C. for 10 min, then warmed to room temperature and stirred another 15 min at this temperature. LCMS indicated that the starting material was completely consumed, and the desired mass was detected. The reaction mixture was poured into saturated aqueous NH₄Cl (10 mL) and extracted with DCM (10 mL×4). The combined organic layers were dried over Na₂SO₄, filtered, and concentrated under vacuum to give the crude (1-benzyl-4,4-dimethylazetidin-2-yl)methanol (75.0 mg, 365 μmol, 53% yield) as a yellow gum. M+H⁺=206.1 (LCMS); ¹H NMR (400 MHZ, DMSO-d₆) δ 7.35-7.16 (m, 5H), 4.18-4.03 (m, 1H), 3.66 (d, J=13.6 Hz, 1H), 3.49 (d, J=13.5 Hz, 1H), 3.23-3.06 (m, 3H), 1.78 (dd, J=7.4, 10.1 Hz, 1H), 1.66-1.56 (m, 1H), 1.19 (s, 3H), 0.98 (s, 3H).

### Step 6: 5-((1-Benzyl-4,4-dimethylazetidin-2-yl)methoxy)-2-methyl-N-(1-(naphthalen-1-yl)cyclopropyl)benzamide (Compound 492) and 5-((1-benzyl-5,5-dimethylpyrrolidin-3-yl)oxy)-2-methyl-N-(1-(naphthalen-1-yl)cyclopropyl)benzamide (Compound 491)

A mixture of 5-hydroxy-2-methyl-N-(1-(naphthalen-1-yl)cyclopropyl)benzamide (100 mg, 315 μmol, 1.0 eq), (1-benzyl-4,4-dimethylazetidin-2-yl)methanol (64.7 mg, 315 μmol, 1.0 eq), and CMBP (114 mg, 473 μmol, 1.5 eq) in toluene (5.0 mL) was degassed and purged with N₂ three times. The resulting mixture was stirred at 100° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×4). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 50%-70% B over 8 min; mobile phase A: 10 mM aqueous NH₄HCO₃, mobile phase B: acetonitrile). 5-((1-Benzyl-4,4-dimethylazetidin-2-yl)methoxy)-2-methyl-N-(1-(naphthalen-1-yl)cyclopropyl)benzamide (16.2 mg, 31.8 μmol, 10% yield) was obtained as a white solid. M+H⁺=505.3 (LCMS); ¹H NMR (400 MHZ, DMSO-d₆) δ 9.04 (s, 1H), 8.66 (d, J=8.3 Hz, 1H), 7.93 (d, J=7.8 Hz, 1H), 7.82 (t, J=6.6 Hz, 2H), 7.59-7.43 (m, 3H), 7.26 (d, J=7.1 Hz, 2H), 7.13 (t, J=7.4 Hz, 2H), 7.08-7.01 (m, 1H), 6.96 (d, J=8.5 Hz, 1H), 6.65 (dd, J=2.6, 8.4 Hz, 1H), 6.42 (d, J=2.8 Hz, 1H), 3.74-3.63 (m, 2H), 3.60-3.40 (m, 3H), 1.94 (s, 3H), 1.86 (dd, J=7.6, 10.1 Hz, 1H), 1.67 (dd, J=8.2, 9.8 Hz, 1H), 1.34 (br s, 2H), 1.23-1.13 (m, 5H), 1.00 (s, 3H). 5-((1-Benzyl-5,5-dimethylpyrrolidin-3-yl)oxy)-2-methyl-N-(1-(naphthalen-1-yl)cyclopropyl)benzamide (6.50 mg, 12.8 μmol, 4% yield) was obtained as a yellow solid. M+H⁺=505.3 (LCMS); ¹H NMR (400 MHZ, DMSO-d₆) δ 9.04 (s, 1H), 8.63 (br d, J=7.9 Hz, 1H), 7.93 (br d, J=7.1 Hz, 1H), 7.86-7.72 (m, 2H), 7.62-7.38 (m, 3H), 7.36-7.12 (m, 5H), 6.97 (br d, J=8.3 Hz, 1H), 6.71 (br dd, J=2.1, 8.3 Hz, 1H), 6.46 (br d, J=2.1 Hz, 1H), 4.72 (br s, 1H), 3.57 (br d, J=13.0 Hz, 1H), 3.46-3.35 (m, 1H), 3.32 (s, 2H), 2.96-2.76 (m, 1H), 2.10 (br dd, J=8.0, 12.8 Hz, 1H), 1.93 (s, 3H), 1.71 (br dd, J=2.9, 13.0 Hz, 1H), 1.33 (br s, 2H), 1.16 (s, 4H), 1.05 (s, 3H).

### Example 291: 5-((4,4-Dimethylazetidin-2-yl)methoxy)-2-methyl-N-(1-(naphthalen-1-yl)cyclopropyl)benzamide (Compound 534)

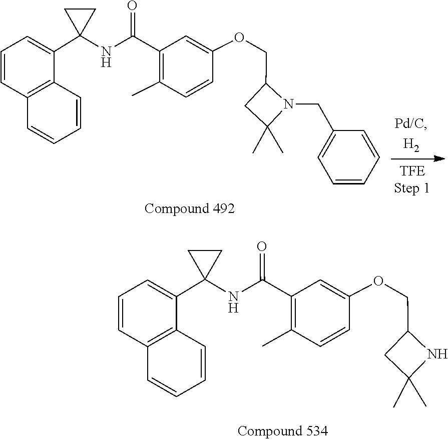 [SMILES: Cc1ccc(OCC2CC(C)(C)N2Cc2ccccc2)cc1C(=O)NC1(c2cccc3ccccc23)CC1 ; Cc1ccc(OCC2CC(C)(C)N2)cc1C(=O)NC1(c2cccc3ccccc23)CC1]

### Step 1: 5-((4,4-Dimethylazetidin-2-yl)methoxy)-2-methyl-N-(1-(naphthalen-1-yl)cyclo propyl)benzamide (Compound 534)

To a solution of 5-((1-benzyl-4,4-dimethylazetidin-2-yl)methoxy)-2-methyl-N-(1-(naphthalen-1-yl)cyclopropyl)benzamide (110 mg, 218 μmol, 1.0 eq) in TFE (20 mL) was added 10% palladium on carbon (100 mg) under a N₂ atmosphere. The suspension was degassed and purged with H₂ several times. The mixture was stirred at 20° C. for 16 h under a H₂ (15 psi) atmosphere. LCMS indicated that the starting material was completely consumed, and the desired mass was detected. The suspension was filtered through a pad of Celite, and the filter cake was washed with EtOAc (10 mL×3). The combined organic layers were concentrated under vacuum to give a residue which was purified by preparative HPLC (Waters Xbridge BEH C18 column (100×30 mm, 10 μm); flow rate: 25 mL/min; gradient: 30%-55% B over 8 min; mobile phase A: 10 mM aqueous NH₄HCO₃, mobile phase B: acetonitrile). 5-((4,4-Dimethylazetidin-2-yl)methoxy)-2-methyl-N-(1-(naphthalen-1-yl)cyclopropyl)benzamide (7.20 mg, 17.4 μmol, 8% yield) was obtained as a white solid. M+H⁺=415.1 (LCMS); 1H NMR (400 MHZ, DMSO-d₆) δ 9.07 (s, 1H), 8.66 (d, J=8.5 Hz, 1H), 7.93 (d, J=8.0 Hz, 1H), 7.82 (t, J=8.3 Hz, 2H), 7.60-7.42 (m, 3H), 7.01 (d, J=8.4 Hz, 1H), 6.81 (dd, J=2.7, 8.3 Hz, 1H), 6.59 (d, J=2.6 Hz, 1H), 3.90-3.72 (m, 3H), 2.03 (dd, J=7.9, 10.7 Hz, 1H), 1.96 (s, 3H), 1.76 (dd, J=7.3, 10.8 Hz, 1H), 1.38-1.33 (m, 2H), 1.30 (s, 3H), 1.17 (br t, J=5.4 Hz, 2H), 1.13 (s, 3H).

### Example 292: 5-((5,5-Dimethylpyrrolidin-3-yl)oxy)-2-methyl-N-(1-(naphthalen-1-yl)cyclopropyl)benzamide (Compound 533)

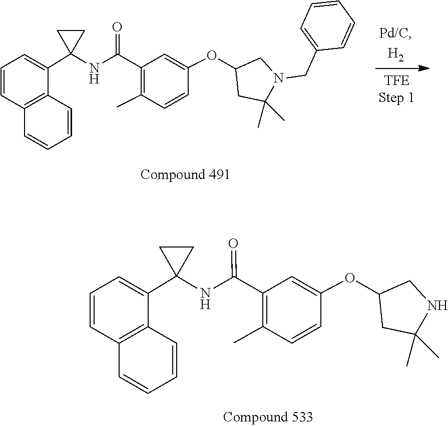 [SMILES: Cc1ccc(OC2CN(Cc3ccccc3)C(C)(C)C2)cc1C(=O)NC1(c2cccc3ccccc23)CC1 ; Cc1ccc(OC2CNC(C)(C)C2)cc1C(=O)NC1(c2cccc3ccccc23)CC1]

### Step 1: 5-((5,5-Dimethylpyrrolidin-3-yl)oxy)-2-methyl-N-(1-(naphthalen-1-yl)cyclopropyl)benzamide (Compound 533)

To a solution of 5-((1-benzyl-5,5-dimethylpyrrolidin-3-yl)oxy)-2-methyl-N-(1-(naphthalen-1-yl)cyclopropyl)benzamide (100 mg, 198 μmol, 1.0 eq) in TFE (20 mL) was added 10% palladium on carbon (100 mg) under a N₂ atmosphere. The suspension was degassed under vacuum and purged with H₂ several times. The mixture was stirred at 20° C. for 16 h under a H₂ (15 psi) atmosphere. LCMS indicated that the starting material was completely consumed, and the desired mass was detected. The suspension was filtered through a pad of Celite, and the filter cake was washed with EtOAc (10 mL×3). The mixture was concentrated under vacuum to give a residue which was purified by preparative HPLC (Waters Xbridge BEH C18 column (100×30 mm, 10 μm); flow rate: 25 mL/min; gradient: 30%-60% B over 8 min; mobile phase A: 10 mM aqueous NH₄HCO₃, mobile phase B: acetonitrile). 5-((5,5-Dimethylpyrrolidin-3-yl)oxy)-2-methyl-N-(1-(naphthalen-1-yl)cyclopropyl)benzamide (21.8 mg, 52.2 μmol, 26% yield) was obtained as a white solid. M+H⁺=415.1 (LCMS); ¹H NMR (400 MHZ, DMSO-d₆) δ 9.07 (s, 1H), 8.66 (br d, J=8.0 Hz, 1H), 7.93 (br d, J=7.9 Hz, 1H), 7.81 (br dd, J=7.7, 11.2 Hz, 2H), 7.60-7.42 (m, 3H), 7.01 (br d, J=8.6 Hz, 1H), 6.76 (br d, J=7.8 Hz, 1H), 6.51 (br s, 1H), 4.75 (br s, 1H), 3.19-3.11 (m, 1H), 2.87-2.78 (m, 1H), 1.96 (s, 3H), 1.84 (br dd, J=7.4, 13.4 Hz, 1H), 1.54 (br d, J=13.3 Hz, 1H), 1.35 (br s, 2H), 1.17 (br s, 2H), 1.12 (s, 3H), 1.06 (s, 3H).

### Example 293:4-Amino-2-methyl-5-((1-methylazetidin-2-yl)methoxy)-N-(1-(naphthalen-1-yl)cyclopropyl)benzamide (Compound 487)

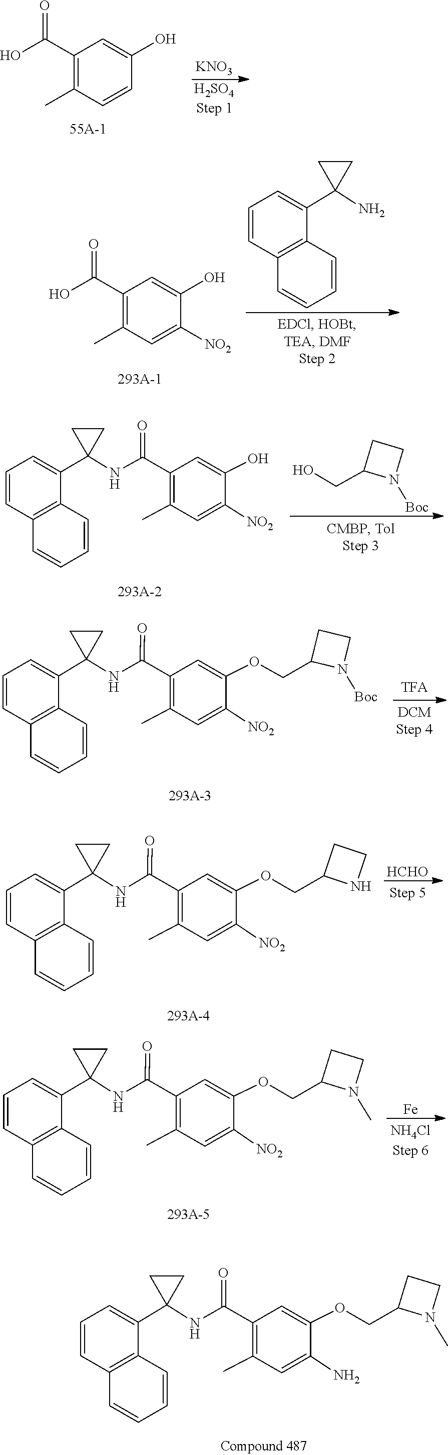

### Step 1: 5-Hydroxy-2-methyl-4-nitrobenzoic acid (293A-1)

A solution of 5-hydroxy-2-methylbenzoic acid (5.00 g, 32.9 mmol, 1.0 eq) in H₂SO₄ (50 mL, 98% purity) was cooled to 0° C. and to this mixture was added KNO₃ (3.49 g, 34.5 mmol, 1.1 eq) at 0° C. The mixture was stirred at 20° C. for 1 h. TLC indicated that the starting material was completely consumed, and a main spot was detected. The mixture was treated with ice water (40 mL) slowly and extracted with DCM (20 mL×5). The combined organic layers were dried over Na₂SO₄, filtered, and concentrated under vacuum to give a residue which was purified by flash silica gel chromatography using a gradient of EtOAc/petroleum ether from 0/1 to 1/0. 5-Hydroxy-2-methyl-4-nitrobenzoic acid (640 mg, 3.25 mmol, 10% yield) was obtained as a yellow solid. ¹H NMR (400 MHZ, CDCl₃) δ 10.29-10.19 (m, 1H), 8.07-7.99 (m, 1H), 7.90-7.80 (m, 1H), 2.67-2.58 (m, 3H).

### Step 2: 5-Hydroxy-2-methyl-N-(1-(naphthalen-1-yl)cyclopropyl)-4-nitrobenzamide (293A-2)

To a solution of 5-hydroxy-2-methyl-4-nitrobenzoic acid (600 mg, 3.04 mmol, 1.0 eq) and 1-(naphthalen-1-yl)cyclopropanamine (502 mg, 2.74 mmol, 0.90 eq) in DMF (30 mL) were added TEA (308 mg, 3.04 mmol, 424 μL, 1.0 eq), EDCI (613 mg, 3.20 mmol, 1.1 eq) and HOBt (82.3 mg, 609 μmol, 0.20 eq). The mixture was stirred at 20° C. for 16 h. LCMS indicated that the starting material was completely consumed, and the desired mass was detected. The reaction mixture was poured into brine (50 mL) and extracted with EtOAc (10 mL×5). The combined organic layers were dried over Na₂SO₄, filtered, and concentrated under vacuum to give a residue which was purified by flash silica gel chromatography using a gradient of EtOAc/petroleum ether from 0/1 to 1/1. 5-Hydroxy-2-methyl-N-(1-(naphthalen-1-yl)cyclopropyl)-4-nitrobenzamide (460 mg, 1.27 mmol, 42% yield) was obtained as a yellow solid. M+H⁺=363.1 (LCMS); ¹H NMR (400 MHZ, CDCl₃) δ 10.41-10.24 (m, 1H), 8.48-8.40 (m, 1H), 7.96-7.89 (m, 2H), 7.86-7.80 (m, 2H), 7.64-7.44 (m, 3H), 6.93-6.89 (m, 1H), 6.53-6.44 (m, 1H), 2.14-2.09 (m, 3H), 1.63-1.57 (m, 2H), 1.47-1.40 (m, 2H).

### Step 3: tert-Butyl 2-((4-methyl-5-((1-(naphthalen-1-yl)cyclopropyl)carbamoyl)-2-nitrophenoxy)methyl)azetidine-1-carboxylate (293A-3)

To a mixture of 5-hydroxy-2-methyl-N-(1-(naphthalen-1-yl)cyclopropyl)-4-nitrobenzamide (140 mg, 386 μmol, 1.0 eq) and ter-butyl 2-(hydroxymethyl)azetidine-1-carboxylate (72.3 mg, 386 μmol, 1.0 eq) in toluene (8.0 mL) was added CMBP (933 mg, 5.42 mmol, 3.0 eq). The mixture was degassed and purged with N₂ three times. The mixture was stirred at 100° C. for 14 h under a N₂ atmosphere. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. tert-Butyl 2-((4-methyl-5-((1-(naphthalen-1-yl)cyclopropyl)carbamoyl)-2-nitrophenoxy)methyl)azetidine-1-carboxylate (90.0 mg, 169 μmol, 44% yield) was obtained as a yellow oil. M+H⁺=532.3 (LCMS); ¹H NMR (400 MHZ, DMSO-d₆) δ 9.28 (s, 1H), 8.61 (d, J=8.3 Hz, 1H), 7.94 (d, J=7.8 Hz, 1H), 7.83 (dd, J=7.7, 11.7 Hz, 2H), 7.69 (s, 1H), 7.61-7.43 (m, 3H), 7.04 (s, 1H), 4.45-4.32 (m, 2H), 4.11 (br d, J=8.3 Hz, 1H), 3.70 (br s, 2H), 2.34-2.21 (m, 1H), 2.17-2.05 (m, 1H), 1.92 (s, 3H), 1.43-1.21 (m, 13H).

### Step 4: 5-(Azetidin-2-ylmethoxy)-2-methyl-N-(1-(naphthalen-1-yl)cyclopropyl)-4-nitro benzamide (293A-4)

To a solution of tert-butyl 2-((4-methyl-5-((1-(naphthalen-1-yl)cyclopropyl)carbamoyl)-2-nitrophenoxy)methyl)azetidine-1-carboxylate (70.0 mg, 132 μmol, 1.0 eq) in DCM (3.0 mL) was added TFA (1.08 g, 9.45 mmol, 700 μL, 72 eq). The mixture was stirred at 20° C. for 1 h. LCMS indicated that the starting material was completely consumed, and the desired mass was detected. The mixture was concentrated under vacuum at 20° C. to give the crude product 5-(azetidin-2-ylmethoxy)-2-methyl-N-(1-(naphthalen-1-yl)cyclopropyl)-4-nitrobenzamid (90.0 mg, TFA salt) as a yellow oil. M+H⁺=432.1 (LCMS).

### Step 5: 2-Methyl-5-((1-methylazetidin-2-yl)methoxy)-N-(1-(naphthalen-1-yl)cyclopropyl)-4-nitrobenzamide (293A-5)

To a solution of 5-(azetidin-2-ylmethoxy)-2-methyl-N-(1-(naphthalen-1-yl)cyclopropyl)-4-nitrobenzamide (90.0 mg, 165 μmol, 1.0 eq) in MeOH (4.0 mL) was added TEA (23.0 μL), followed by the addition of formaldehyde (26.8 mg, 330 μmol, 24.6 μL, 37% purity in water, 2.0 eq). The resulting mixture was adjusted to pH 6 with a small amount of AcOH. The mixture was stirred at 20° C. for 30 min, then NaBH₃CN (20.7 mg, 330 μmol, 2.0 eq) was added. The reaction mixture was stirred at 20° C. for another 14 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative TLC (DCM/MeOH=10/1, R_{f}=0.4). 2-Methyl-5-((1-methylazetidin-2-yl)methoxy)-N-(1-(naphthalen-1-yl)cyclopropyl)-4-nitrobenzamide (60.0 mg, 135 μmol, 82% yield) was obtained as a yellow solid. M+H⁺=446.1 (LCMS).

### Step 6:4-Amino-2-methyl-5-((1-methylazetidin-2-yl)methoxy)-N-(1-(naphthalen-1-yl)cyclopropyl)benzamide (Compound 487)

To a stirred solution of 2-methyl-5-((1-methylazetidin-2-yl)methoxy)-N-(1-(naphthalen-1-yl)cyclopropyl)-4-nitrobenzamide (60.0 mg, 135 μmol, 1.0 eq) in a mixture of MeOH (6.0 mL) and H₂O (1.2 mL) was added iron powder (37.6 mg, 673 μmol, 5.0 eq), followed by NH₄Cl (36.0 mg, 673 μmol, 5.0 eq). The mixture was stirred at 70° C. for 14 h. LCMS indicated that the starting material was completely consumed, and the desired mass was detected. The reaction mixture was allowed to cool to room temperature, filtered through a pad of Celite. The filtrate was poured into H₂O (10 mL) and extracted with EtOAc (3.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 5%-35% B over 8 min; mobile phase A: 0.04% aqueous HCl, mobile phase B: acetonitrile). 4-Amino-2-methyl-5-((1-methylazetidin-2-yl)methoxy)-N-(1-(naphthalen-1-yl)cyclopropyl)benzamide (24.0 mg, 52.3 μmol, 39% yield, HCl salt) was obtained as a white solid. M+H⁺=416.1 (LCMS); ¹H NMR (400 MHZ, DMSO-d₆) δ 8.93 (br s, 1H), 8.67 (d, J=8.4 Hz, 1H), 7.93 (d, J=7.8 Hz, 1H), 7.85-7.80 (m, 2H), 7.60-7.43 (m, 3H), 6.84-6.70 (m, 2H), 4.71-4.60 (m, 1H), 4.31-4.18 (m, 2H), 4.05 (dt, J=4.4, 9.7 Hz, 1H), 3.90-3.82 (m, 1H), 2.83 (s, 3H), 2.48-2.30 (m, 2H), 1.98 (s, 3H), 1.36 (br s, 2H), 1.18 (br s, 2H).

### Example 294: 5-((Azetidin-3-yloxy)methyl)-2-methyl-N-(1-(naphthalen-1-yl)cyclopropyl)benzamide (Compound 435)

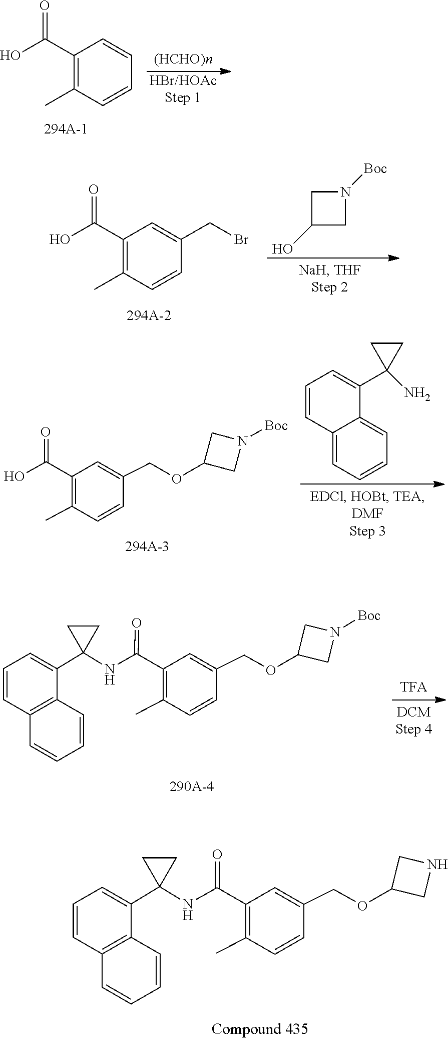

### Step 1: 5-(Bromomethyl)-2-methylbenzoic acid (294A-2)

To a solution of 2-methylbenzoic acid (4.00 g, 29.4 mmol, 3.77 mL, 1.0 eq) in phosphoric acid (0.5 mL) were added paraformaldehyde (2.50 g) and HBr (19.4 g, 79.0 mmol, 13.0 mL, 33% purity in HOAc, 2.7 eq) at 20° C. The mixture was stirred at 115° C. for 15 h under a N₂ atmosphere. TLC indicated that the starting material was completely consumed, and a main spot was detected. The reaction mixture was allowed to cool to room temperature, poured into H₂O (20 mL). The resulting solid was collected by filtration, washed with H₂O (20 mL) and dried under vacuum to give a crude product 5-(bromomethyl)-2-methylbenzoic acid (6.00 g) as a white solid. M+H⁺=229.0 (LCMS).

### Step 2: 5-(((1-(tert-Butoxycarbonyl)azetidin-3-yl)oxy)methyl)-2-methylbenzoic acid (294A-3)

To a solution of 5-(bromomethyl)-2-methylbenzoic acid (300 mg, 1.31 mmol, 1.0 eq) in THF (10 mL) was added NaH (105 mg, 2.62 mmol, 60% purity, 2.0 eq). The mixture was stirred at 0° C. for 30 min, then tert-butyl 3-hydroxyazetidine-1-carboxylate (340 mg, 1.96 mmol, 1.5 eq) was added at 0° C. The mixture was stirred at 0° C. for 2 h. LCMS indicated that 3% of the starting material was remaining and 66% of desired compound was detected. The mixture was treated with H₂O (10 mL) and extracted with DCM (10 mL×2). The organic phase was discarded. The aqueous layer was acidified to pH 5 by using HCl (1 M aqueous) and extracted with DCM (10 mL×3). The combined organic layers were dried over Na₂SO₄, filtered, and concentrated under vacuum to give residue which was purified by preparative HPLC (C18-1 column (150×30 mm, 5 μm); flow rate: 25 mL/min; gradient: 35%-80%, B over 8 min; mobile phase A: 0.1% aqueous TFA, mobile phase B: acetonitrile). (((1-(tert-Butoxycarbonyl)azetidin-3-yl)oxy)methyl)-2-methylbenzoic acid (1.10 g, 3.22 mmol, 49% yield) was obtained as a white solid. M−100+H⁺=222.2 (LCMS).

### Step 3: tert-Butyl 3-((4-methyl-3-((1-(naphthalen-1-yl)cyclopropyl)carbamoyl)benzyl)oxy)azetidine-1-carboxylate (294A-4)

To a solution of 1-(naphthalen-1-yl)cyclopropanamine (214 mg, 1.17 mmol, 1.5 eq) in DCM (10 mL) were added HOBt (263 mg, 1.94 mmol, 2.5 eq), TEA (236 mg, 2.33 mmol, 325 μL, 3.0 eq), EDCI (373 mg, 1.94 mmol, 2.5 eq) and 5-(((1-(tert-butoxycarbonyl)azetidin-3-yl)oxy)methyl)-2-methylbenzoic acid (250 mg, 778 μmol, 1.0 eq). The mixture was stirred at 20° C. for 16 h. LCMS indicated that the starting material was completely consumed. The mixture was concentrated under vacuum to give a residue which was purified by preparative HPLC (Phenomenex Gemini C18 column (150×30 mm, 5 μm); flow rate: 25 mL/min;

gradient: 35%-80% B over 8 min; mobile phase A: 0.1% aqueous TFA, mobile phase B: acetonitrile). tert-Butyl 3-((4-methyl-3-((1-(naphthalen-1-yl)cyclopropyl)carbamoyl)benzyl)oxy)azetidine-1-carboxylate (250 mg, 488 μmol, 63% yield) was obtained as a white amorphous solid. M−100+H⁺=387.2 (LCMS).

### Step 4: 5-((Azetidin-3-yloxy)methyl)-2-methyl-N-(1-(naphthalen-1-yl)cyclopropyl)benzamide (Compound 435)

To a solution of tert-butyl 3-((4-methyl-3-((1-(naphthalen-1-yl)cyclopropyl)carbamoyl)benzyl)oxy)azetidine-1-carboxylate (100 mg, 206 μmol, 1.0 eq) in DCM (1.0 mL) was added TFA (23.4 mg, 206 μmol, 15.2 μL, 1.0 eq). The mixture was stirred at 20° C. for 1 h. LCMS indicated that the starting material was completely consumed. The mixture was concentrated under vacuum to give a residue which was purified by preparative HPLC (Phenomenex Luna C18 column (75×30 mm, 3 μm); flow rate: 25 mL/min; gradient: 10%-45% B over 8 min; mobile phase A: 0.1% aqueous TFA, mobile phase B: acetonitrile). 5-((Azetidin-3-yloxy)methyl)-2-methyl-N-(1-(naphthalen-1-yl)cyclopropyl)benzamide (23.5 mg, 46.9 μmol, 23% yield, TFA salt) was obtained as a white solid. M+H⁺=387.1 (LCMS); ¹H NMR (400 MHz, DMSO-d₆) δ9.23-9.07 (m, 1H), 8.71-8.52 (m, 2H), 7.99-7.80 (m, 3H), 7.65-7.44 (m, 3H), 7.34-6.79 (m, 3H), 4.47-4.32 (m, 3H), 4.15-4.03 (m, 2H), 3.79 (m, 2H), 2.03 (m, 1H), 1.98-1.94 (m, 1H), 2.09-1.93 (m, 1H), 1.37 (m, 2H), 1.26-1.12 (m, 2H).

### Example 295: 2-Methyl-5-(((1-methylazetidin-3-yl)oxy)methyl)-N-(1-(naphthalen-1-yl)cyclopropyl)benzamide (Compound 447)

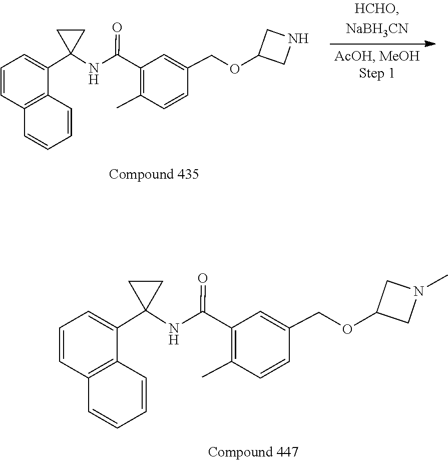 [SMILES: Cc1ccc(COC2CNC2)cc1C(=O)NC1(c2cccc3ccccc23)CC1 ; Cc1ccc(COC2CN(C)C2)cc1C(=O)NC1(c2cccc3ccccc23)CC1]

### Step 1: 2-Methyl-5-(((1-methylazetidin-3-yl)oxy)methyl)-N-(1-(naphthalen-1-yl)cyclopropyl)benzamide (Compound 447)

To a solution of 5-((azetidin-3-yloxy)methyl)-2-methyl-N-(1-(naphthalen-1-yl)cyclopropyl)benzamide (80.0 mg, 207 μmol, 1.0 eq, TFA salt) in MeOH (2.0 mL) were added formaldehyde (16.8 mg, 207 μmol, 15.4 μL, 37% purity in water, 1.0 eq), TEA (20.9 mg, 207 μmol, 28.8 μL, 1.0 eq) and HOAc (1.24 mg, 20.7 μmol, 1.18 μL, 0.10 eq). The mixture was stirred at 20° C. for 1 h. Then NaBH₃CN (32.5 mg, 517 μmol, 2.5 eq) was added to the mixture. The resulting mixture was stirred at 20° C. for 11 h. LCMS indicated that the starting material was completely consumed. The mixture was concentrated under vacuum to give a residue which was purified by preparative HPLC (Waters Xbridge Prep OBD C18 column (150×40 mm, 10 μm); flow rate: 25 mL/min; gradient: 20%-50% B over 8 min; mobile phase A: 10 mM aqueous NH₄HCO₃, mobile phase B: acetonitrile). 2-Methyl-5-(((1-methylazetidin-3-yl)oxy)methyl)-N-(1-(naphthalen-1-yl)cyclopropyl)benzamide (16.3 mg, 40.7 μmol, 20% yield) was obtained as a white solid. M+H⁺=401.1 (LCMS); ¹H NMR (400 MHZ, CDCl₃) δ 7.64-7.55 (m, 1H), 7.54-7.44 (m, 2H), 7.21-7.13 (m, 1H), 7.13-7.03 (m, 2H), 6.50 (m, 1H), 4.32-4.31 (m, 1H), 4.33-4.25 (m, 2H), 4.16-4.03 (m, 1H), 3.63-3.50 (m, 2H), 2.87 (m, 2H), 2.34 (m, 3H), 2.17 (m, 3H), 1.63-1.52 (m, 2H), 1.45-1.34 (m, 2H).

### Example 296: 5-(2-(Azetidin-2-yl)ethyl)-2-methyl-N-(1-(naphthalen-1-yl)cyclopropyl)benzamide (Compound 449)

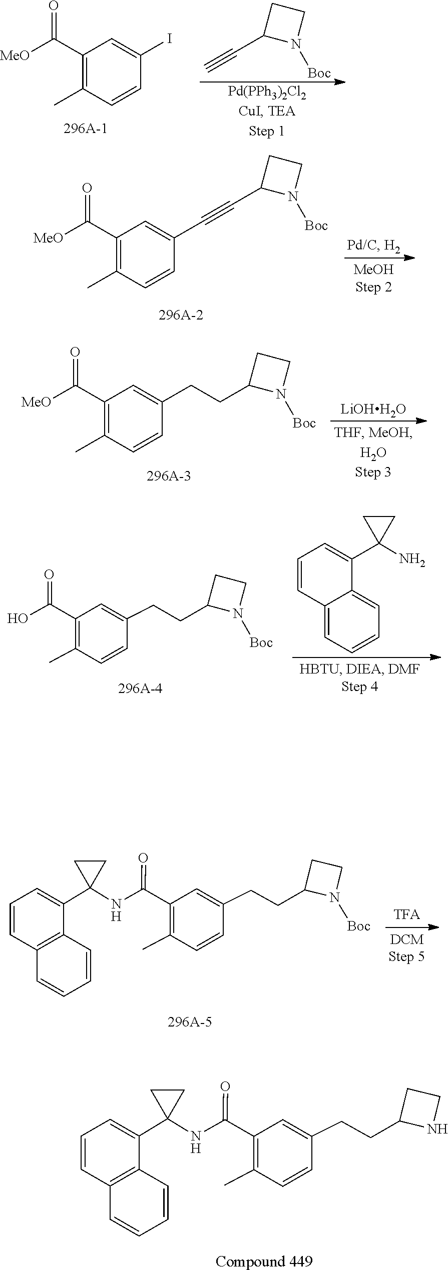

### Step 1: tert-Butyl 2-((3-(methoxycarbonyl)-4-methylphenyl) ethynyl)azetidine-1-carboxylate (296A-2)

To a solution of tert-butyl 2-ethynylazetidine-1-carboxylate (270 mg, 1.49 mmol, 1.0 eq) in TEA (5.0 mL) were added methyl 5-iodo-2-methylbenzoate (411 mg, 1.49 mmol, 1.0 eq), CuI (5.67 mg, 29.8 μmol, 0.02 eq), and Pd(PPh₃)₂Cl₂ (20.9 mg, 29.8 μmol, 0.02 eq). The mixture was stirred at 20° C. for 16 h under a N₂ atmosphere. LCMS indicated that the starting material was completely consumed, and the desired mass was detected. The mixture was treated with water (10 mL) and extracted with DCM (10 mL×5). The combined organic layers were dried over Na₂SO₄, filtered, and concentrated under vacuum to give a residue which was purified by flash silica gel chromatography using a gradient of EtOAc/petroleum ether from 0/1 to 1/3. tert-Butyl 2-((3-(methoxycarbonyl)-4-methyl phenyl) ethynyl)azetidine-1-carboxylate (220 mg, 655 μmol, 44% yield) was obtained as a colorless oil. M−100+H⁺=230.1 (LCMS).

### Step 2: tert-Butyl 2-(3-(methoxycarbonyl)-4-methylphenethyl)azetidine-1-carboxylate (296A-3)

To a solution of tert-butyl 2-((3-(methoxycarbonyl)-4-methylphenyl) ethynyl)azetidine-1-carboxylate (200 mg, 607 μmol, 1.0 eq) in MeOH (10 mL) was added 10% palladium on carbon (30.0 mg). The mixture was stirred at 50° C. for 2 h under a H₂ (15 psi) atmosphere. LCMS indicated that the starting material was completely consumed, and the desired mass was detected. The reaction mixture was allowed to cool to room temperature and filtered through a pad of Celite and the filtrate was concentrated under vacuum to give a crude product tert-butyl 2-(3-(methoxycarbonyl)-4-methylphenethyl)azetidine-1-carboxylate (200 mg) as a colorless oil. M−100+H⁺=234.1 (LCMS).

### Step 3: 5-(2-(1-(tert-Butoxycarbonyl)azetidin-2-yl)ethyl)-2-methylbenzoic acid (296A-4)

To a solution of tert-butyl 2-(3-(methoxycarbonyl)-4-methylphenethyl)azetidine-1-carboxy late (200 mg, 599 μmol, 1.0 eq) in THF (1.0 mL) were added LiOH·H₂O (176 mg, 4.20 mmol, 7.0 eq), MeOH (500 μL) and H₂O (500 μL). The mixture was stirred at 20° C. for 1 h. LCMS indicated that the starting material was completely consumed, and the desired mass was detected. The mixture was treated with H₂O (5.0 mL) and extracted with DCM (5.0 mL×2). The organic phase was discarded. The aqueous layer was acidified to pH 5 by using HCl (1 M aqueous) and extracted with DCM (5.0 mL×3). The combined organic layers were dried over Na₂SO₄, filtered, and concentrated under vacuum to give a crude product 5-(2-(1-(tert-butoxycarbonyl)azetidin-2-yl)ethyl)-2-methylbenzoic acid (200 mg) as a colorless oil. M−100+H⁺=220.1 (LCMS).

### Step 4: tert-Butyl 2-(4-methyl-3-((1-(naphthalen-1-yl)cyclopropyl)carbamoyl)phenethyl)azetidine-1-carboxylate (296A-5)

To a solution of 1-(naphthalen-1-yl)cyclopropanamine (86.1 mg, 469 μmol, 1.0 eq) in DCM (2.0 mL) were added HOBt (159 mg, 1.17 mmol, 2.5 eq), TEA (143 mg, 1.41 mmol, 196 μL, 3.0 eq), EDCI (225 mg, 1.17 mmol, 2.5 eq) and 5-(2-(1-(tert-butoxycarbonyl)azetidin-2-yl)ethyl)-2-methylbenzoic acid (150 mg, 469 μmol, 1.0 eq). The mixture was stirred at 20° C. for 16 h. LCMS indicated that the starting material was completely consumed, and the desired mass was detected. The mixture was treated with water (10 mL) and extracted with DCM (5.0 mL×5). The combined organic layers were dried over Na₂SO₄, filtered, and concentrated under vacuum to give a crude product tert-butyl 2-(4-methyl-3-((1-(naphthalen-1-yl)cyclopropyl)carbamoyl)phenethyl)azetidine-1-carboxylate (400 mg) as a yellow oil. M−100+H⁺=385.2 (LCMS).

### Step 5: 5-(2-(Azetidin-2-yl)ethyl)-2-methyl-N-(1-(naphthalen-1-yl)cyclopropyl)benz amide (Compound 449)

To a solution of tert-butyl 2-(4-methyl-3-((1-(naphthalen-1-yl)cyclopropyl)carbamoyl) phenethyl)azetidine-1-carboxylate (300 mg, 619 μmol, 1.0 eq) in DCM (3.0 mL) was added TFA (70.6 mg, 619 μmol, 45.8 μL, 1.0 eq). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Luna C18 column (150×30 mm, 5 μm); flow rate: 25 mL/min; gradient: 15%-40% B over 8 min; mobile phase A: 0.1% aqueous TFA, mobile phase B: acetonitrile). 5-(2-(Azetidin-2-yl)ethyl)-2-methyl-N-(1-(naphthalen-1-yl)cyclopropyl)benzamide (66.0 mg, 130 μmol, 21% yield, TFA salt) was obtained as a white solid. M+H⁺=385.1 (LCMS); ¹H NMR (400 MHZ, DMSO-d₆) δ 9.11-9.04 (m, 1H), 8.68-8.63 (m, 1H), 7.97-7.91 (m, 1H), 7.87-7.81 (m, 2H), 7.61-7.43 (m, 3H), 7.12-7.04 (m, 2H), 6.97-6.88 (m, 1H), 4.28-4.16 (m, 1H), 3.96-3.80 (m, 1H), 3.76-3.65 (m, 1H), 2.47-2.28 (m, 4H), 2.21-2.09 (m, 1H), 2.08 (m, 1H), 1.99 (m, 3H), 1.96-1.85 (m, 1H), 1.39-1.32 (m, 2H).

### Example 297: 2-Methyl-5-(2-(1-methylazetidin-2-yl)ethyl)-N-(1-(naphthalen-1-yl)cyclo

propyl)benzamide (Compound 455)

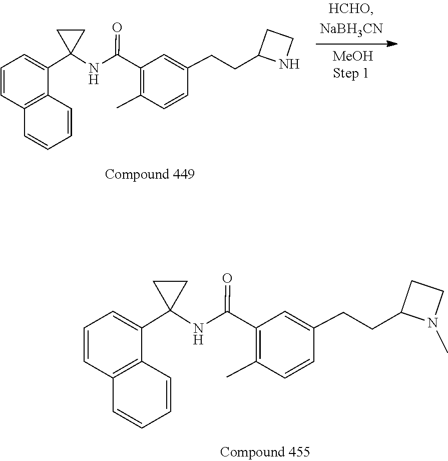 [SMILES: Cc1ccc(CCC2CCN2)cc1C(=O)NC1(c2cccc3ccccc23)CC1 ; Cc1ccc(CCC2CCN2C)cc1C(=O)NC1(c2cccc3ccccc23)CC1]

### Step 1: 2-Methyl-5-(2-(1-methylazetidin-2-yl)ethyl)-N-(1-(naphthalen-1-yl)cyclo propyl)benzamide (Compound 455)

To a solution of 5-(2-(azetidin-2-yl)ethyl)-2-methyl-N-(1-(naphthalen-1-yl)cyclopropyl)benzamide (40.0 mg, 104 μmol, 1.0 eq, TFA salt) in MeOH (1.0 mL) were added formaldehyde (8.44 mg, 104 μmol, 7.74 μL, 37% purity in water, 1.0 eq), TEA (10.5 mg, 104 μmol, 14.5 μL, 1.0 eq) and HOAc (624 μg, 10.4 μmol, 0.1 eq). The mixture was stirred at 20° C. for 1 h. Then NaBH₃CN (16.3 mg, 260 μmol, 2.5 eq) was added to the mixture. The mixture was stirred at 20° C. for 15 h. LCMS indicated that the starting material was completely consumed. The mixture was concentrated under vacuum to give a residue which was purified by preparative HPLC (Waters Xbridge Prep OBD C18 column (150×40 mm, 10 μm); flow rate: 25 mL/min; gradient: 20%-55% B over 8 min; mobile phase A: 10 mM aqueous NH₄HCO₃, mobile phase B: acetonitrile). 2-Methyl-5-(2-(1-methylazetidin-2-yl)ethyl)-N-(1-(naphthalen-1-yl)cyclopropyl)benzamide (20.8 mg, 52.2 μmol, 50% yield) was obtained as a white solid. M+H⁺=399.1 (LCMS); ¹H NMR (400 MHZ, CDCl₃) δ 8.52-8.44 (m, 1H), 7.98-7.88 (m, 2H), 7.84-7.77 (m, 1H), 7.61-7.43 (m, 3H), 7.08-6.94 (m, 3H), 6.53-6.44 (m, 1H), 3.42-3.33 (m, 1H), 2.95-2.83 (m, 1H), 2.76-2.67 (m, 1H), 2.51-2.42 (m, 2H), 2.27 (m, 3H), 2.15 (m, 3H), 2.00-1.91 (m, 1H), 1.88-1.74 (m, 2H), 1.73-1.62 (m, 1H), 1.62-1.55 (m, 2H), 1.44-1.37 (m, 2H).

### Example 298: Methyl 3-(4-methyl-3-((1-(naphthalen-1-yl)cyclopropyl)carbamoyl)phenyl) propanoate (Compound 431)

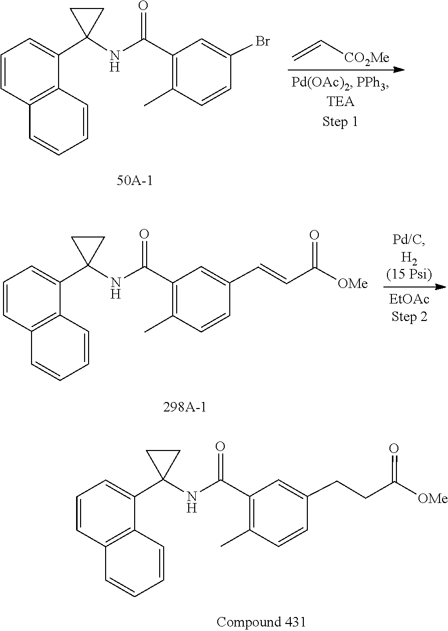

### Step 1: (E)-Methyl 3-(4-methyl-3-((1-(naphthalen-1-yl)cyclopropyl)carbamoyl)phenyl)acrylate (298A-1)

To a solution of 5-bromo-2-methyl-N-(1-(naphthalen-1-yl)cyclopropyl)benzamide (120 mg, 316 μmol, 1.0 eq), Pd(OAc) 2 (708 mg, 3.16 μmol, 0.01 eq) and PPh₃ (1.66 mg, 6.31 μmol, 0.02 eq) in DMF (6.0 mL) was added methyl acrylate (272 mg, 3.16 mmol, 284 μl, 10 eq), followed by TEA (63.9 mg, 631 μmol, 87.8 μL, 2.0 eq) under a N₂ atmosphere. The mixture was stirred at 100° C. for 16 h. LCMS indicated that the starting material was completely consumed. The reaction mixture was allowed to cool to room temperature, poured into H₂O (3.0 mL) and extracted with EtOAc (3.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/2. (E)-Methyl 3-(4-methyl-3-((1-(naphthalen-1-yl)cyclopropyl)carbamoyl)phenyl) acrylate (104 mg, 270 μmol, 29% yield) was obtained as a yellow oil. M+H⁺=386.1 (LCMS).

### Step 2: Methyl 3-(4-methyl-3-((1-(naphthalen-1-yl)cyclopropyl)carbamoyl)phenyl)propanoate (Compound 431)

To a solution of (E)-methyl 3-(4-methyl-3-((1-(naphthalen-1-yl)cyclopropyl)carbamoyl)phenyl) acrylate (90.0 mg, 234 μmol, 1.0 eq) in EtOAc (10 mL) was added 10% palladium on carbon (10 mg). The mixture was degassed and purged with H₂ three times, and then the mixture was stirred at 20° C. for 2 h under a H₂ (15 psi) atmosphere. LCMS indicated that the starting material was completely consumed. The suspension was filtered through a pad of Celite, and the filter cake was washed with EtOAc (2.0 mL×3). The combined filtrates were concentrated under vacuum to give a residue which was purified by preparative HPLC (Phenomenex Luna column (80×30 mm, 3 μm); flow rate: 25 mL/min; gradient: 35%-65% B over 8 min; mobile phase A: 0.04% aqueous HCl, mobile phase B: acetonitrile). Methyl 3-(4-methyl-3-((1-(naphthalen-1-yl)cyclopropyl)carbamoyl)phenyl) propanoate (21.5 mg, 54.2 μmol, 24% yield, HCl salt) was obtained as a yellow solid. M+H⁺=388.1 (LCMS); ¹H NMR (400 MHZ, DMSO-d₆) δ 9.10-9.01 (m, 1H), 8.70-8.60 (m, 1H), 7.97-7.89 (m, 1H), 7.87-7.77 (m, 2H), 7.61-7.42 (m, 3H), 7.13-7.07 (m, 1H), 7.06-6.99 (m, 1H), 6.91 (d, J=1.3 Hz, 1H), 3.52 (s, 3H), 2.79-2.70 (m, 2H), 2.56 (s, 2H), 2.01-1.93 (m, 3H), 1.40-1.31 (m, 2H), 1.21-1.12 (m, 2H).

### Example 299: 5-(3-Amino-3-oxopropyl)-2-methyl-N-(1-(naphthalen-1-yl)cyclopropyl)benzamide (Compound 452)

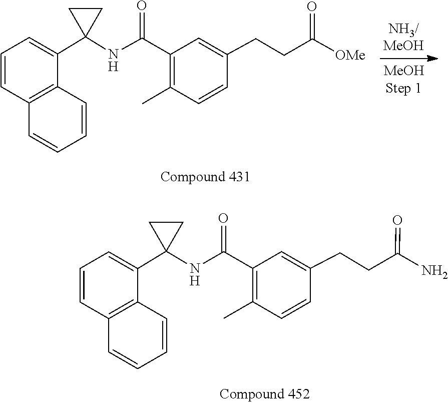 [SMILES: COC(=O)CCc1ccc(C)c(C(=O)NC2(c3cccc4ccccc34)CC2)c1 ; Cc1ccc(CCC(N)=O)cc1C(=O)NC1(c2cccc3ccccc23)CC1]

### Step 1: 5-(3-Amino-3-oxopropyl)-2-methyl-N-(1-(naphthalen-1-yl)cyclopropyl)benzamide (Compound 452)

To a solution of methyl 3-(4-methyl-3-((1-(naphthalen-1-yl)cyclopropyl)carbamoyl)phenyl) propanoate (320 mg, 826 μmol, 1.0 eq) in MeOH (3.0 mL) was added NH₃/MeOH (7 M, 32 mL, 271 eq). The mixture was stirred at 70° C. for 16 h. LCMS indicated that the starting material was completely consumed, and the desired mass was detected. The reaction mixture was allowed to cool to room temperature and concentrated under vacuum to give a residue which was purified by preparative HPLC (Waters Xbridge BEH C18 column (100×25 mm, 5 μm); flow rate: 40 mL/min; gradient: 20%-50% B over 8 min; mobile phase A: 10 mM aqueous NH₄HCO₃, mobile phase B: acetonitrile). 5-(3-Amino-3-oxopropyl)-2-methyl-N-(1-(naphthalen-1-yl)cyclopropyl)benzamide (25.9 mg, 69.5 μmol, 8% yield) was obtained as a white solid. M+H⁺=373.1 (LCMS); ¹H NMR (400 MHZ, DMSO-d₆) δ 9.06 (s, 1H), 8.71-8.60 (m, 1H), 7.98-7.90 (m, 1H), 7.83 (d, J=7.9 Hz, 2H), 7.63-7.42 (m, 3H), 7.27-7.18 (m, 1H), 7.05 (br d, J=17.9 Hz, 2H), 6.90 (s, 1H), 6.77-6.64 (m, 1H), 2.76-2.63 (m, 2H), 2.32-2.20 (m, 2H), 1.97 (s, 3H), 1.35 (br s, 2H), 1.23-1.11 (m, 2H).

### Example 300: 5-(3-Aminopropyl)-2-methyl-N-(1-(naphthalen-1-yl)cyclopropyl)benzamide (Compound 472)

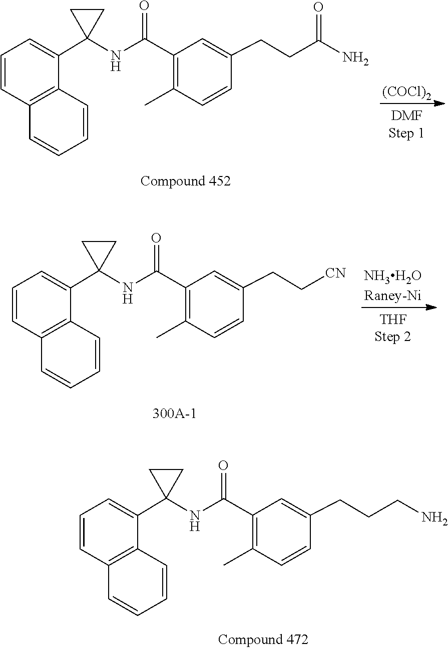

### Step 1: 5-(2-Cyanoethyl)-2-methyl-N-(1-(naphthalen-1-yl)cyclopropyl)benzamide (300A-1)

A mixture of 5-(3-amino-3-oxopropyl)-2-methyl-N-(1-(naphthalen-1-yl)cyclopropyl)benz amide (68.0 mg, 183 μmol, 1.0 eq) in DMF (5.0 mL) was degassed and purged with N₂ three times, and to this solution was added (COCl)₂ (69.5 mg, 548 μmol, 47.9 μL, 3.0 eq) at 0° C. The mixture was stirred at 0° C. for 2 h under a N₂ atmosphere. LCMS indicated that the starting material was completely consumed, and the desired mass was detected. The reaction mixture was poured into H₂O (6.0 mL) and extracted with EtOAc (4.0 mL×3). The combined organic layers were dried over Na₂SO₄, filtered, and concentrated under vacuum to give a residue which was purified by preparative TLC (EtOAc/petroleum ether=1/3, R_{f}=0.5). Crude 5-(2-cyanoethyl)-2-methyl-N-(1-(naphthalen-1-yl)cyclopropyl)benzamide (76.0 mg) was obtained as a yellow oil. M+H⁺=355.2 (LCMS).

### Step 2: 5-(3-Aminopropyl)-2-methyl-N-(1-(naphthalen-1-yl)cyclopropyl)benzamide (Compound 472)

To a solution of 5-(2-cyanoethyl)-2-methyl-N-(1-(naphthalen-1-yl)cyclopropyl)benzamide (61.0 mg, 172 μmol, 1.0 eq) in THF (20 mL) was added Raney-Ni (14.7 mg), followed by NH₃·H₂O (24.1 mg, 172 μmol, 26.5 μL, 25% purity 1.0 eq). The mixture was degassed and purged with H₂ three times. The mixture was stirred at 30° C. for 16 h under a H₂ (50 psi) atmosphere. LCMS indicated that the starting material was completely consumed, and the desired mass was detected. The reaction mixture was filtered through a pad of Celite and the filtrate was concentrated under vacuum to give a residue which was purified by preparative HPLC (Phenomenex Luna column (80×30 mm, 3 μm); flow rate: 25 mL/min; gradient: 10%-60% B over 8 min; mobile phase A: 0.04% aqueous HCl, mobile phase B: acetonitrile). 5-(3-Aminopropyl)-2-methyl-N-(1-(naphthalen-1-yl)cyclopropyl)benzamide (9.00 mg, 22.7 μmol, 13% yield, HCl salt) was obtained as a white solid. M+H⁺=359.1 (LCMS); ¹H NMR (400 MHz, DMSO-d₆) δ 9.09 (s, 1H), 8.71-8.61 (m, 1H), 7.93 (d, J=7.4 Hz, 1H), 7.87-7.80 (m, 2H), 7.73 (br s, 3H), 7.48 (d, J=7.5 Hz, 3H), 7.12-7.04 (m, 2H), 6.93-6.88 (m, 1H), 2.77-2.63 (m, 2H), 2.58-2.52 (m, 2H), 1.99 (s, 3H), 1.80-1.69 (m, 2H), 1.35 (br d, J=1.5 Hz, 2H), 1.18 (br d, J=1.4 Hz, 2H).

### Example 301: 5-(4-Aminopiperidin-1-yl)-2-methyl-N-(1-(naphthalen-1-yl)cyclopropyl)benzamide (Compound 463)

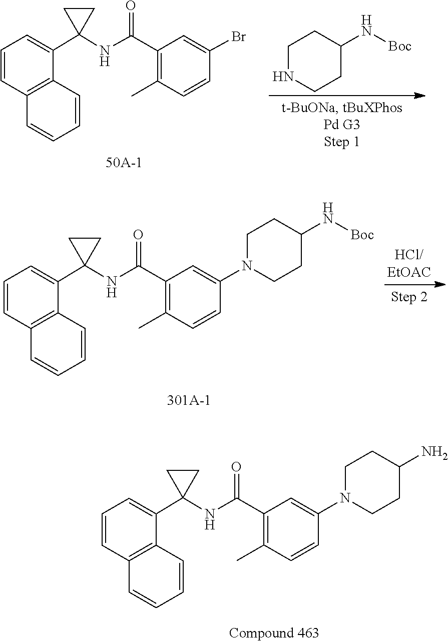 [SMILES: Cc1ccc(Br)cc1C(=O)NC1(c2cccc3ccccc23)CC1 ; CC(C)(C)OC(=O)NC1CCNCC1 ; Cc1ccc(N2CCC(NC(=O)OC(C)(C)C)CC2)cc1C(=O)NC1(c2cccc3ccccc23)CC1 ; Cc1ccc(N2CCC(N)CC2)cc1C(=O)NC1(c2cccc3ccccc23)CC1]

### Step 1: tert-Butyl(1-(4-methyl-3-((1-(naphthalen-1-yl)cyclopropyl)carbamoyl)phenyl)piperidin-4-yl)carbamate (301A-1)

A mixture of 5-bromo-2-methyl-N-(1-(naphthalen-1-yl)cyclopropyl)benzamide (100 mg, 263 μmol, 1.0 eq) and tert-butyl piperidin-4-ylcarbamate (79.0 mg, 394 μmol, 4.38 μL, 1.5 eq) in THF (4.0 mL) was degassed and purged with N₂ three times. To this mixture were added t-BuONa (75.8 mg, 789 μmol, 3.0 eq) and t-BuXPhos Pd G3 (20.9 mg, 26.3 μmol, 0.1 eq). The mixture was stirred at 80° C. for 16 h under a N₂ atmosphere. LCMS indicated that the starting material was completely consumed, and the desired mass was detected. The reaction mixture was poured into H₂O (10 mL) and extracted with DCM (10 mL×3). The combined organic layers were dried over Na₂SO₄, filtered, and concentrated under vacuum to give a residue which was purified by flash silica gel chromatography using a gradient of EtOAc/petroleum ether from 0/1 to 3/5 tert-Butyl(1-(4-methyl-3-((1-(naphthalen-1-yl)cyclopropyl)carbamoyl)phenyl) piperidin-4-yl)carbamate (80.0 mg, 160 μmol, 20% yield) was obtained as a brown solid. M+H⁺=500.3 (LCMS).

### Step 2: 5-(4-Aminopiperidin-1-yl)-2-methyl-N-(1-(naphthalen-1-yl)cyclopropyl)benz amide (Compound 463)

To a solution of tert-butyl(1-(4-methyl-3-((1-(naphthalen-1-yl)cyclopropyl)carbamoyl)phenyl) piperidin-4-yl)carbamate (75.0 mg, 150 μmol, 1.0 eq) in EtOAc (5.0 mL) was added HCl/EtOAc (4 M, 7.5 mL). The resulting mixture was stirred at 25° C. for 30 min. LCMS indicated that the starting material was completely consumed, and the desired mass was detected. The reaction mixture was concentrated under vacuum at 30° C. to give a residue which was purified by preparative HPLC (Phenomenex Luna C18 column (80×30 mm, 3 μm); flow rate: 25 mL/min; gradient: 1%-35% B over 8 min; mobile phase A: 0.04% aqueous HCl, mobile phase B: acetonitrile). 5-(4-Aminopiperidin-1-yl)-2-methyl-N-(1-(naphthalen-1-yl)cyclopropyl)benzamide (13.0 mg, 32.5 μmol, 22% yield, HCl salt) was obtained as a white solid. M+H⁺=400.1 (LCMS); ¹H NMR (400 MHZ, DMSO-d₆) δ 9.08 (br s, 1H), 8.66 (d, J=8.4 Hz, 1H), 8.09-7.98 (m, 3H), 7.93 (d, J=8.4 Hz, 1H), 7.82 (dd, J=7.9, 11.1 Hz, 2H), 7.63-7.39 (m, 3H), 7.02 (br d, J=3.9 Hz, 1H), 6.92-6.57 (m, 1H), 3.58 (br d, J=12.5 Hz, 2H), 3.25-3.08 (m, 1H), 2.94-2.63 (m, 2H), 1.93 (s, 5H), 1.77-1.44 (m, 2H), 1.35 (br s, 2H), 1.18 (br s, 2H).

### Example 302: 5-(4-(2-Hydroxyethyl)piperazin-1-yl)-2-methyl-N-(1-(naphthalen-1-yl)cyclopropyl)benzamide (Compound 505)

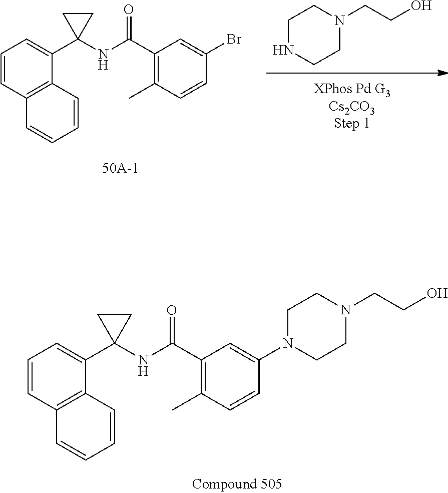 [SMILES: Cc1ccc(Br)cc1C(=O)NC1(c2cccc3ccccc23)CC1 ; OCCN1CCNCC1 ; Cc1ccc(N2CCN(CCO)CC2)cc1C(=O)NC1(c2cccc3ccccc23)CC1]

### Step 1: 5-(4-(2-Hydroxyethyl)piperazin-1-yl)-2-methyl-N-(1-(naphthalen-1-yl)cyclopropyl)benzamide (Compound 505)

A mixture of 5-bromo-2-methyl-N-(1-(naphthalen-1-yl)cyclopropyl)benzamide (150 mg, 394 μmol, 1.0 eq) and 2-(piperazin-1-yl)ethanol (61.6 mg, 473 μmol, 58.0 μL, 1.2 eq) in tert-amylalcohol (3.0 mL) was degassed and purged with N₂ three times. To the mixture were added XPhos Pd G3 (33.4 mg, 39.4 μmol, 0.1 eq) and Cs₂CO₃ (257 mg, 788 μmol, 2.0 eq). The resulting mixture was stirred at 80° C. for 16 h under a N₂ atmosphere. LCMS indicated that the starting material was completely consumed, and the desired mass was detected. The reaction mixture was allowed to cool to room temperature, poured into H₂O (5.0 mL) and extracted with DCM (2.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 10%-35% B over 8 min; mobile phase A: 0.04% aqueous HCl, mobile phase B: acetonitrile). 5-(4-(2-Hydroxyethyl)piperazin-1-yl)-2-methyl-N-(1-(naphthalen-1-yl)cyclopropyl)benzamide (3.30 mg, 2.92 μmol, HCl salt) was obtained as a white solid. M+H⁺=430.1 (LCMS); ¹H NMR (400 MHZ, DMSO-d₆) δ 9.68-9.55 (m, 1H), 9.03 (s, 1H), 8.66 (d, J=8.5 Hz, 1H), 7.93 (d, J=7.4 Hz, 1H), 7.86-7.79 (m, 2H), 7.60-7.43 (m, 3H), 7.02 (d, J=8.0 Hz, 1H), 6.93-6.86 (m, 1H), 6.64 (d, J=2.8 Hz, 1H), 3.80-3.72 (m, 2H), 3.70-3.64 (m, 2H), 3.57-3.53 (m, 2H), 3.24-3.20 (m, 2H), 3.16-3.07 (m, 2H), 3.01-2.90 (m, 2H), 1.93 (s, 3H), 1.38-1.32 (m, 2H), 1.21-1.14 (m, 2H).

### Example 303: 5-Methyl-2-((methylamino)methyl)-N-(1-(naphthalen-1-yl)cyclopropyl)benzo[d]oxazole-6-carboxamide (Compound 426)

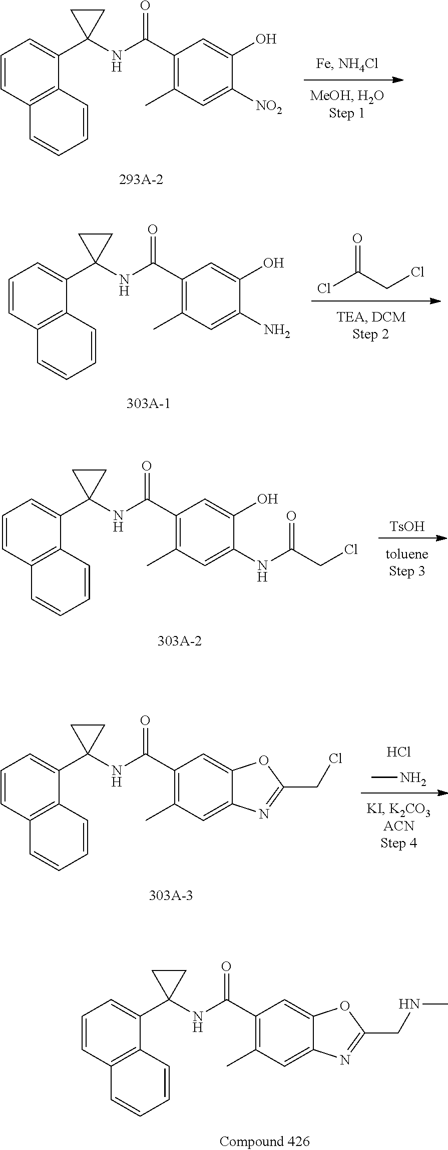

### Step 1:4-Amino-5-hydroxy-2-methyl-N-(1-(naphthalen-1-yl)cyclopropyl)benzamide (301A-1)

To a stirred solution of 5-hydroxy-2-methyl-N-(1-(naphthalen-1-yl)cyclopropyl)-4-nitrobenz amide (230 mg, 635 μmol, 1.0 eq) in a mixture of MeOH (8.0 mL) and H₂O (1.6 mL) was added iron powder (355 mg, 6.35 mmol, 10 eq) in one portion, followed by NH₄Cl (340 mg, 6.35 mmol, 10 eq). The mixture was stirred at 70° C. for 3 h. LCMS indicated that the starting material was completely consumed, and the desired mass was detected. The reaction mixture was allowed to cool to room temperature, poured into H₂O (5.0 mL), filtered, and the filtrate was extracted with DCM (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/0. 4-Amino-5-hydroxy-2-methyl-N-(1-(naphthalen-1-yl)cyclopropyl)benzamide (130 mg, 391 μmol, 31% yield) was obtained as a yellow solid. M+H⁺=333.1 (LCMS).

### Step 2:4-(2-Chloroacetamido)-5-hydroxy-2-methyl-N-(1-(naphthalen-1-yl)cyclopropyl)benzamide (301A-2)

To a solution of 4-amino-5-hydroxy-2-methyl-N-(1-(naphthalen-1-yl)cyclopropyl)benzamide (120 mg, 361 μmol, 1.0 eq) in DCM (4.0 mL) was added TEA (110 mg, 1.08 mmol, 151 μL, 3.0 eq) at 0° C., followed by 2-chloroacetyl chloride (28.5 mg, 253 μmol, 20.1 μL, 0.7 eq) in DCM (1.0 mL). The resulting mixture was stirred at 20° C. for 2 h. LCMS indicated that the starting material was consumed, and the desired mass was detected. The mixture was treated with water (10 mL) and extracted with DCM (5.0 mL×5). The combined organic layers were dried over Na₂SO₄, filtered, and concentrated under vacuum to give a residue which was purified via preparative TLC (EtOAc/petroleum ether=1/1, R_{f}=0.4). 4-(2-Chloroacetamido)-5-hydroxy-2-methyl-N-(1-(naphthalen-1-yl)cyclopropyl)benzamide (40.0 mg, 97.8 μmol, 27% yield) was obtained as a yellow solid. M+H⁺=409.1 (LCMS); ¹H NMR (400 MHZ, CDCl₃) δ 8.52-8.41 (m, 1H), 7.96-7.79 (m, 3H), 7.64-7.41 (m, 4H), 7.10-7.04 (m, 1H), 6.87-6.77 (m, 1H), 6.63-6.47 (m, 1H), 5.39-5.25 (m, 1H), 4.26-4.19 (m, 2H), 2.14 (br d, J=2.1 Hz, 3H), 1.40-1.39 (m, 2H), 1.30-1.29 (m, 2H).

### Step 3: 2-(Chloromethyl)-5-methyl-N-(1-(naphthalen-1-yl)cyclopropyl)benzo[d]oxazole-6-carboxamide (301A-3)

To a solution of 4-(2-chloroacetamido)-5-hydroxy-2-methyl-N-(1-(naphthalen-1-yl)cyclopropyl)benzamide (60.0 mg, 147 μmol, 1.0 eq) in toluene (6.0 mL) was added TsOH·H₂O (12.6 mg, 73.4 μmol, 0.5 eq). The resulting mixture was stirred at 100° C. for 16 h. LCMS indicated that the starting material was completely consumed. The mixture was allowed to cool to room temperature, poured into H₂O (5.0 mL) and extracted with DCM (5.0 mL×5). The combined organic layers were dried over Na₂SO₄, filtered, and concentrated under vacuum to give a residue which was purified via preparative TLC (EtOAc/petroleum ether=1/3, R_{f}=0.5). 2-(Chloromethyl)-5-methyl-N-(1-(naphthaalen-1-yl)cyclopropyl)benzo[d]oxazole-6-carboxamide (20.0 mg, 51.2 μmol, 35% yield) was obtained as a yellow solid. M+H⁺=391.1 (LCMS).

### Step 4: 5-Methyl-2-((methylamino)methyl)-N-(1-(naphthalen-1-yl)cyclopropyl)benzo[d]oxazole-6-carboxamide (Compound 426)

To a solution of 2-(chloromethyl)-5-methyl-N-(1-(naphthaalen-1-yl)cyclopropyl)benzo[d]oxazole-6-carboxamide (20.0 mg, 51.2 μmol, 1.0 eq) in acetonitrile (2.0 mL) was added methanamine hydrochloride (13.8 mg, 205 μmol, 4.0 eq), followed by KI (1.70 mg, 10.2 μmol, 0.20 eq) and K₂CO₃ (14.1 mg, 102 μmol, 2.0 eq). The mixture was stirred at 80° C. for 16 h.

LCMS indicated that the starting material was completely consumed. The mixture was allowed to cool to room temperature, poured into H₂O (10 mL) and extracted with DCM (5.0 mL×5). The combined organic layers were dried over Na₂SO₄, filtered, and concentrated under vacuum to give a residue which was purified by preparative HPLC (Phenomenex Luna C18 column (75× 30 mm, 3 μm); flow rate: 25 mL/min; gradient: 15%-45% B over 8 min; mobile phase A:

0.2% aqueous FA, mobile phase B: acetonitrile). 5-Methyl-2-((methylamino)methyl)-N-(1-(naphthalen-1-yl)cyclopropyl)benzo[d]oxazole-6-carboxamide (3.20 mg, 7.20 μmol, 14% yield, FA salt) was obtained as a white solid. M+H⁺=386.1 (LCMS); ¹H NMR (400 MHZ, DMSO-d₆) δ 9.20-9.14 (m, 1H), 8.68-8.61 (m, 1H), 7.98-7.89 (m, 1H), 7.87-7.78 (m, 2H), 7.62-7.43 (m, 4H), 7.38-7.34 (m, 1H), 3.91-3.86 (m, 2H), 2.30-2.26 (m, 3H), 2.13-2.09 (m, 3H), 1.42-1.36 (m, 2H), 1.23-1.17 (m, 2H).

### Example 304: N-(1-(2,3-Dihydrobenzo[b][1,4]dioxin-5-yl)cyclopropyl)-2-methyl-5-((1-methylazetidin-2-yl)methoxy)benzamide (Compound 432)

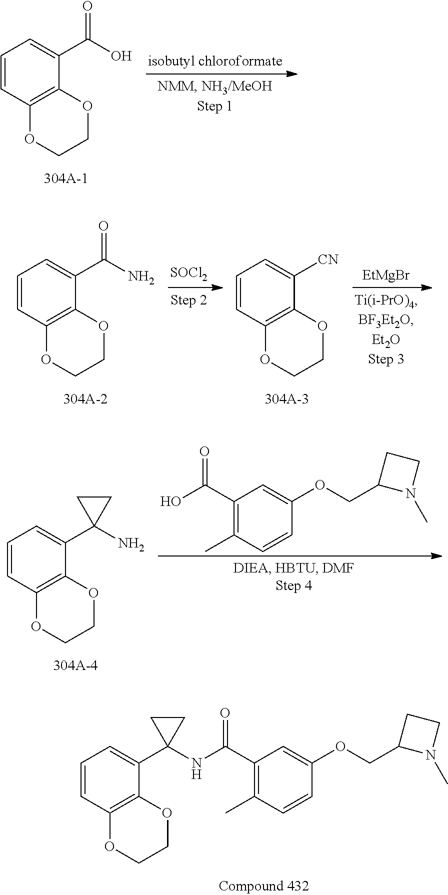

### Step 1:2,3-Dihydrobenzo[b][1,4]dioxine-5-carboxamide (304A-2)

To a solution of 2,3-dihydrobenzo[b][1,4]dioxine-5-carboxylic acid (2.00 g, 11.1 mmol, 1.0 eq) in THF (50 mL) were added NMM (1.24 g, 12.2 mmol, 1.34 mL 1.1 eq) and isobutyl chloroformate (1.67 g, 12.2 mmol, 1.60 mL, 1.1 eq) at 0° C. under a N₂ atmosphere. The mixture was stirred at 0° C. for 30 min. NH₃/MeOH (7 M, 10 mL, 6.3 eq) was added slowly. The mixture was stirred at 20° C. for 1 h. TLC indicated that the starting material was completely consumed. The mixture was treated with H₂O (40 mL) and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 0/1 to 100/1. 2,3-Dihydrobenzo[b][1,4]dioxine-5-carboxamide (1.80 g, 10.1 mmol, 90% yield) was obtained as a white solid. ¹H NMR (400 MHZ, DMSO-d₆) δ 7.52 (br s, 2H), 7.29 (dd, J=1.7, 7.7 Hz, 1H), 6.97 (dd, J=1.7, 7.9 Hz, 1H), 6.91-6.82 (m, 1H), 4.38-4.32 (m, 2H), 4.30-4.24 (m, 2H).

### Step 2:2,3-Dihydrobenzo[b][1,4]dioxine-5-carbonitrile (304A-3)

A solution of 2,3-dihydrobenzo[b][1,4]dioxine-5-carboxamide (500 mg, 2.79 mmol, 1.0 eq) in SOCl₂ (2.5 mL) was stirred at 80° C. for 6 h. TLC indicated that the starting material was completely consumed, and a main spot was detected. The reaction mixture was allowed to cool to room temperature, poured into H₂O (5.0 mL) and extracted with DCM (3.0 mL×3). The combined organic layers were dried over Na₂SO₄, filtered, and concentrated under vacuum to give crude product which was purified by flash silica gel chromatography using a gradient of EtOAc/petroleum ether from 0/1 to 100/1. 2,3-Dihydrobenzo[b][1,4]dioxine-5-carbonitrile (300 mg, 1.86 mmol, 67% yield) was obtained as a white solid. ¹H NMR (400 MHZ, DMSO-d₆) δ 7.32-7.15 (m, 2H), 7.03-6.92 (m, 1H), 4.47-4.38 (m, 2H), 4.37-4.29 (m, 2H).

### Step 3: 1-(2,3-Dihydrobenzo[b][1,4]dioxin-5-yl)cyclopropanamine (304A-4)

A mixture of 2,3-dihydrobenzo[b][1,4]dioxine-5-carbonitrile (150 mg, 931 μmol, 1.0 eq) in anhydrous Et₂O (15 mL) was degassed and purged with N₂ three times. The mixture was stirred at −78° C. To this mixture was added Ti(i-PrO)₄ (291 mg, 1.02 mmol, 302 μL, 1.5 eq) slowly, and then EtMgBr (3 M in Et₂O, 683 μL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. BF₃·Et₂O (264 mg, 1.86 mmol, 230 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (10 mL) and MTBE (5 mL) and extracted with MTBE (5.0 mL×3). The aqueous layer was basified to pH 8 using NaOH (2 M aqueous), and a precipitate was formed. The mixture was filtered through a pad of Celite and the slurry was washed with DCM several times. The combined filtrate was extracted with DCM (5.0 mL×3). The combined organic layers were dried over Na₂SO₄, filtered, and concentrated under vacuum to give a residue which was purified by preparative TLC (DCM/MeOH=10/1, R_{f}=0.6). 1-(2,3-Dihydrobenzo[b][1,4]dioxin-5-yl)cyclopropane amine (90.0 mg, 471 μmol, 51% yield) was obtained as a brown oil. M+H⁺=192.0 (LCMS).

### Step 4: N-(1-(2,3-Dihydrobenzo[b][1,4]dioxin-5-yl)cyclopropyl)-2-methyl-5-((1-methyl azetidin-2-yl)methoxy)benzamide (Compound 432)

To a solution of 1-(2,3-dihydrobenzo[b][1,4]dioxin-5-yl)cyclopropanamine (50.0 mg, 261 μmol, 1.0 eq) and 2-methyl-5-((1-methylazetidin-2-yl)methoxy)benzoic acid (61.5 mg, 261 μmol, 1.0 eq) in DMF (5.0 mL) were added DIEA (101 mg, 784 μmol, 137 μL, 3.0 eq) and HBTU (99.2 mg, 261 μmol, 1.0 eq). The mixture was stirred at 20° C. for 16 h. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give a residue which was purified by preparative HPLC (Phenomenex Luna column (80× 30 mm, 3 μm); flow rate: 25 mL/min; gradient: 5%-35% B over 8 min; mobile phase A: 0.04% aqueous HCl, mobile phase B: acetonitrile). N-(1-(2,3-Dihydrobenzo[b][1,4]dioxin-5-yl)cyclopropyl)-2-methyl-5-((1-methylazetidin-2-yl)methoxy)benzamide (29.2 mg, 65.6 μmol, 25% yield, HCl salt) was obtained as a white solid. M+H⁺=409.2 (LCMS); ¹H NMR (400 MHZ, DMSO-d₆) δ 10.54-10.40 (m, 1H), 8.66-8.57 (m, 1H), 7.15-7.09 (m, 1H), 7.03 (dd, J=3.0, 6.3 Hz, 1H), 6.92 (dd, J=2.8, 8.4 Hz, 1H), 6.82 (d, J=2.6 Hz, 1H), 6.76-6.69 (m, 2H), 4.64 (dt, J=2.5, 6.9 Hz, 1H), 4.41-4.32 (m, 1H), 4.30-4.27 (m, 2H), 4.25-4.21 (m, 2H), 4.19-4.10 (m, 1H), 4.09-3.97 (m, 1H), 3.93-3.81 (m, 1H), 2.85 (d, J=5.1 Hz, 3H), 2.40-2.30 (m, 2H), 2.13 (s, 3H), 1.09 (br d, J=6.5 Hz, 4H).

### Example 305: N-(1-(2-Isopropylnaphthalen-1-yl)cyclopropyl)-2-methyl-5-((1-methylazetidin-2-yl)methoxy)benzamide (Compound 457)

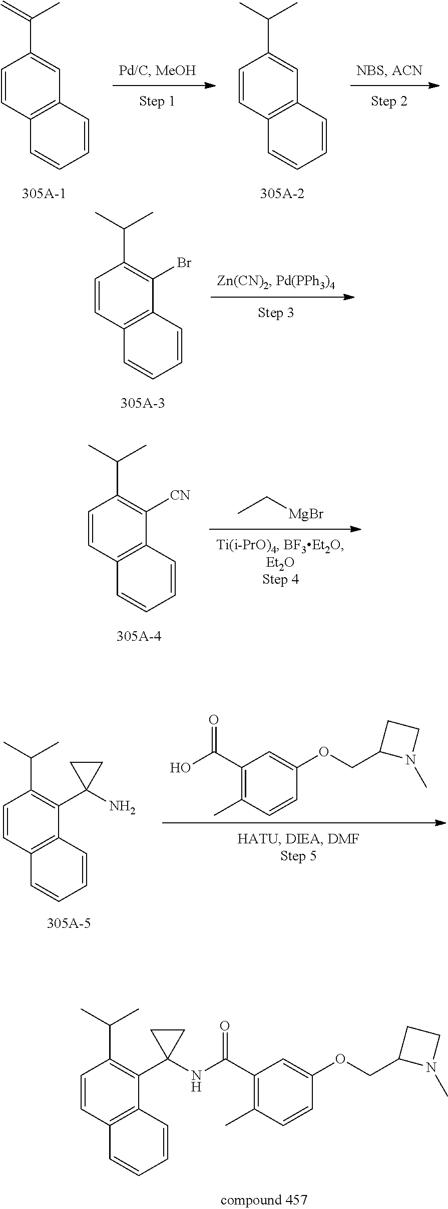

### Step 1: 2-Isopropylnaphthalene (305A-2)

To a mixture of 2-isopropenylnaphthalene (3.00 g, 17.8 mmol, 1.0 eq) in MeOH (20 mL) was added 10% palladium on carbon (3.00 g) at 25° C., the reaction mixture was stirred at 25° C. for 3 h under a H₂ (15 psi) atmosphere. HPLC indicated that the starting material was completely consumed. The reaction mixture was filtered through a pad of Celite and the filtrate was concentrated under vacuum to give a crude product 2-isopropylnaphthalene (3.00 g) as a yellow oil.

### Step 2: 1-Bromo-2-isopropylnaphthalene (305A-3)

To a mixture of 2-isopropylnaphthalene (3.00 g, 17.6 mmol, 1.0 eq) in CH₃CN (60 mL) was added NBS (3.45 g, 19.3 mmol, 1.1 eq) at 0° C., the reaction mixture was stirred at 60° C. for 2 h. HPLC indicated that the starting material was completely consumed. The reaction mixture was allowed to cool to room temperature, poured into saturated aqueous Na₂SO₃ (50 ml), extracted with EtOAc (100 mL×3). The combined organic layers were concentrated under vacuum to give a residue which was purified by preparative HPLC (Phenomenex Luna C18 (75×30 mm, 3 mm); flow rate: 25 mL/min; gradient: 40%-90% B over 10 min; mobile phase A: 0.2% aqueous FA, mobile phase B: acetonitrile). 1-Bromo-2-isopropyl-naphthalene (1.20 g, 4.82 mmol, 27% yield) was obtained as a yellow oil.

### Step 3: 2-Isopropyl-1-naphthonitrile (305A-4)

To a mixture of 1-bromo-2-isopropyl-naphthalene (1.15 g, 4.62 mmol, 1.0 eq) in DMF (40 mL) were added Pd(PPh₃)₄ (533 mg, 461 μmol, 0.1 eq) and Zn(CN)₂ (2.17 g, 18.4 mmol, 1.17 mL, 4.0 eq) at 25° C. The reaction mixture was stirred at 120° C. for 16 h under a N₂ atmosphere. LCMS indicated that the starting material was completely consumed, and the desired mass was detected. The reaction mixture was allowed to cool to room temperature, poured into H₂O (50 ml) and extracted with EtOAc (50 mL×3). The combined organic layers were concentrated under vacuum to give a residue which was purified by preparative HPLC (Phenomenex Luna C18 (75×30 mm, 3 μm); flow rate: 25 mL/min; gradient: 35%-75% B over 8 min; mobile phase A: 0.2% aqueous FA, mobile phase B: acetonitrile). 2-Isopropylnaphthalene-1-carbonitrile (800 mg, 4.10 mmol, δ8% yield) was obtained as a yellow oil. M+H⁺=196.0 (LCMS); ¹H NMR (400 MHZ, CD₃OD) δ 8.10 (d, J=8.4 Hz, 1H), 7.88 (d, J=8.8 Hz, 1H), 7.73 (d, J=8.0 Hz, 1H), 7.52 (dt, J=1.1, 7.6 Hz, 1H), 7.46-7.33 (m, 2H), 3.55 (td, J=6.8, 13.6 Hz, 1H), 1.27 (d, J=6.8 Hz, 6H).

### Step 4: 1-(2-Isopropylnaphthalen-1-yl)cyclopropanamine (305A-5)

A mixture of 2-isopropylnaphthalene-1-carbonitrile (200 mg, 1.02 mmol, 1.0 eq) in anhydrous Et₂O (30 mL) was degassed and purged with N₂ three times. The mixture was stirred at −78° C. To this mixture was added Ti(i-PrO)₄ (320 mg, 1.13 mmol, 332 μL, 1.1 eq) slowly, and then EtMgBr (3 M, 751 μL, 2.2 eq) was added dropwise to maintain the temperature between −78° C. and −75° C. over 1 h under a N₂ atmosphere. After the addition was completed, the resulting mixture was stirred at the same temperature for 10 min and warmed to 20° C. over 1 h. BF₃·Et₂O (290 mg, 2.05 mmol, 252 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (50 mL) and MTBE (30 mL) and extracted with MTBE (30 mL×2). The organic phase was discarded. The aqueous layer was basified to pH 8 by using NaOH (2 M aqueous), and a precipitate was formed. The mixture was filtered through a pad of Celite and the slurry was washed with DCM several times. The combined filtrate was extracted with DCM (50 mL×2). The combined organic layers were dried over Na₂SO₄, filtered, and concentrated under vacuum to give a residue which was purified by preparative HPLC (Phenomenex Luna C18 (10×30 mm, 10 μm); flow rate: 25 mL/min; gradient: 45%-60% B over 8 min; mobile phase A: 10 mM aqueous NH₄HCO₃, mobile phase B: acetonitrile). 1-(2-Isopropyl-1-naphthyl)cyclopropanamine (50.0 mg, 222 μmol, 10% yield) was obtained as a yellow solid. M+H⁺=226.2 (LCMS).

### Step 5: N-(1-(2-Isopropylnaphthalen-1-yl)cyclopropyl)-2-methyl-5-((1-methylazetidin-2-yl)methoxy)benzamide (Compound 457)

To a mixture of 1-(2-isopropyl-1-naphthyl)cyclopropanamine (45.0 mg, 200 μmol, 1.0 eq) and 2-methyl-5-[(1-methylazetidin-2-yl)methoxy]benzoic acid (47.0 mg, 200 μmol, 1.0 eq) in DMF (1.0 mL) were added HATU (114 mg, 299 μmol, 1.5 eq) and DIEA (77.4 mg, 599 μmol, 104 μL, 3.0 eq) at 25° C. The reaction mixture was stirred at 25° C. for 1 h. LCMS indicated that the starting material was completely consumed, and the desired mass was detected. The reaction mixture was filtered, and the filtrate was purified by preparative HPLC (Phenomenex Luna C18 (75×30 mm, 3 μm); flow rate: 25 mL/min; gradient: 15%-50% B over 8 min;

mobile phase A: 10 mM aqueous NH₄HCO₃, mobile phase B: acetonitrile) to give N-(1-(2-isopropylnaphthalen-1-yl)cyclopropyl)-2-methyl-5-((1-methylazetidin-2-yl)methoxy)benzamide (16.5 mg, 36.1 μmol, 18% yield, HCl salt) as a brown solid. M+H⁺=443.2 (LCMS); ¹H NMR (400 MHZ, CD₃OD) δ 9.38 (br s, 1H), 8.02 (d, J=8.0 Hz, 1H), 7.87 (dd, J=8.4, 16.0 Hz, 2H), 7.57 (d, J=8.8 Hz, 1H), 7.53-7.42 (m, 2H), 7.17 (d, J=8.3 Hz, 1H), 7.03-6.92 (m, 2H), 6.84 (q, J=7.2 Hz, 1H), 4.76-4.67 (m, 1H), 4.41-4.30 (m, 1H), 4.28-4.16 (m, 2H), 3.99 (d, J=10.0 Hz, 1H), 3.52-3.43 (m, 1H), 2.98 (s, 3H), 2.65-2.52 (m, 2H), 2.34 (s, 3H), 1.44-1.30 (m, 10H).

### Example 306: 5-(2-Aminopropoxy)-N-(1-(3-methoxynaphthalen-1-yl)cyclopropyl)-2-methylbenzamide (Compound 445)

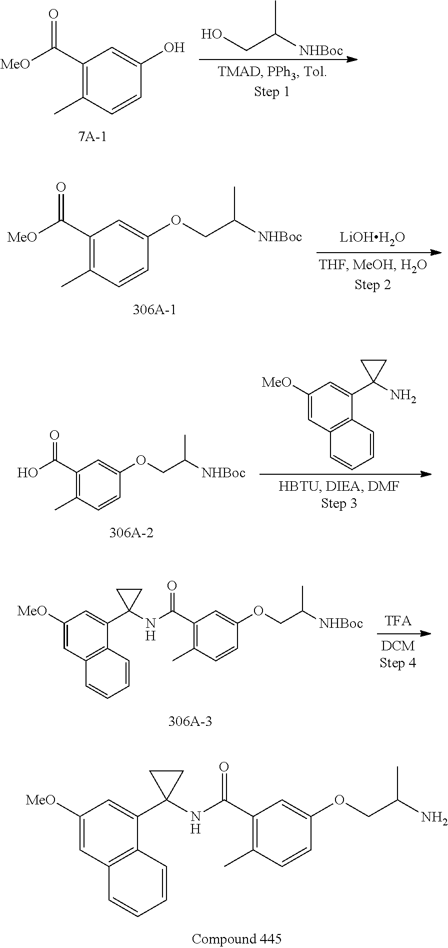

### Step 1: Methyl 5-(2-((tert-butoxycarbonyl)amino)propoxy)-2-methylbenzoate (306A-1)

To a solution of methyl 5-hydroxy-2-methylbenzoate (1.05 g, 6.02 mmol, 1.0 eq) in toluene (20 mL) was added tert-butyl(1-hydroxypropan-2-yl)carbamate (1.00 g, 6.02 mmol, 1.0 eq), followed by TMAD (3.11 g, 18.1 mmol, 3.0 eq) and PPh₃ (4.74 g, 18.1 mmol, 3.0 eq). The mixture was stirred at 100° C. for 16 h under a N₂ atmosphere. LCMS indicated that the starting material remained, and the desired mass was detected. The mixture was allowed to cool room temperature, poured into water (30 mL) and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 0/1 to 6/100. Methyl 5-(2-((tert-butoxycarbonyl)amino)propoxy)-2-methylbenzoate (500 mg, 1.39 mmol, 23% yield) was obtained as a white solid. M−100+H⁺=224.1 (LCMS).

### Step 2: 5-(2-((tert-Butoxycarbonyl)amino)propoxy)-2-methylbenzoic acid (306A-2)

To a solution of methyl 5-(2-((tert-butoxycarbonyl)amino)propoxy)-2-methylbenzoate (618 mg, 1.91 mmol, 1.0 eq) in a mixture of THF (5.0 mL), MeOH (3.5 mL) and H₂O (2.5 mL) was added LiOH·H₂O (246 mg, 5.73 mmol, 3.0 eq). The mixture was stirred at 20° C. for 16 h under a N₂ atmosphere. LCMS indicated that the starting material was completely consumed, and the desired mass was detected. The mixture was concentrated under vacuum at 30° C. to give a residue which was purified by preparative HPLC (Phenomenex luna C18 column (250× 50 mm, 10 μm); flow rate: 60 mL/min; gradient: 40%-80% B over 10 min; mobile phase A: 0.1% TFA, mobile aqueous phase B: acetonitrile). 5-(2-((tert-Butoxycarbonyl)amino)propoxy)-2-methylbenzoic acid (98.0 mg, 317 μmol, 17% yield, TFA salt) was obtained as a white solid. M−100+H⁺=210.2 (LCMS).

### Step 3: tert-Butyl(1-(3-((1-(3-methoxynaphthalen-1-yl)cyclopropyl)carbamoyl)-4-methylphenoxy)propan-2-yl)carbamate (306A-3)

To a solution of 1-(3-methoxynaphthalen-1-yl)cyclopropanamine (20.0 mg, 93.8 μmol, 1.0 eq, TFA salt) in DMF (2.0 mL) was added 5-(2-((tert-butoxycarbonyl)amino)propoxy)-2-methylbenzoic acid (34.8 mg, 113 μmol, 1.2 eq), followed by HBTU (107 mg, 218 mmol, 3.0 eq) and DIEA (121 mg, 938 mmol, 163 μL, 10 eq). The mixture was stirred at 20° C. for 16 h.

LCMS indicated that the starting material remained, and the desired mass was detected. The mixture was treated with water (10 mL) and extracted with EtOAc (5.0 mL×3). The combined organic layers were dried over Na₂SO₄ filtered, and concentrated under vacuum to give a crude product tert-butyl(1-(3-((1-(3-methoxynaphthalen-1-yl)cyclopropyl)carbamoyl)-4-methylphenoxy)propan-2-yl)carbamate (47.0 mg) as a yellow oil. M−56+H⁺=449.2 (LCMS).

### Step 4: 5-(2-Aminopropoxy)-N-(1-(3-methoxynaphthalen-1-yl)cyclopropyl)-2-methylbenzamide (Compound 445)

To a solution of tert-butyl(1-(3-((1-(3-methoxynaphthalen-1-yl)cyclopropyl)carbamoyl)-4-methylphenoxy)propan-2-yl)carbamate (47.0 mg, 93.1 μmol, 1.0 eq) in DCM (2.0 mL) was added TFA (775 mg, 6.80 mmol, 503 μL, 73 eq). The mixture was stirred at 20° C. for 16 h. LCMS indicated that the starting material remained, and the desired mass was detected. The mixture was concentrated under vacuum at 30° C. to give a residue which was purified by preparative HPLC (Phenomenex Luna C18 column (150×30 mm, 5 μm); flow rate: 25 mL/min; gradient: 5%-35% B over 8 min; mobile phase A: 0.1% aqueous TFA, mobile phase B: acetonitrile). 5-(2-Aminopropoxy)-N-(1-(3-methoxynaphthalen-1-yl)cyclopropyl)-2-methylbenzamide (33.4 mg, 64.4 mmol, 41% yield, TFA salt) was obtained as a white solid. M+H⁺=405.1 (LCMS); ¹H NMR (400 MHZ, DMSO-d₆) δ ppm 9.10 (s, 1H), 8.53 (d, J=8.41 Hz, 1H), 7.94 (br s, 2H), 7.83 (d, J=8.16 Hz, 1H), 7.50-7.36 (m, 3H), 7.24 (d, J=2.26 Hz, 1H), 7.08 (d, J=8.41 Hz, 1H), 6.90 (br d, J=2.51 Hz, 1H), 6.67 (d, J=2.51 Hz, 1H), 4.02 (dd, J=10.23, 3.70 Hz, 1H), 3.87 (s, 3H), 3.86-3.81 (m, 1H), 3.54 (br d, J=1.25 Hz, 1H), 1.98 (s, 3H), 1.32 (br s, 2H), 1.22 (d, J=6.65 Hz, 3H), 1.17 (br s, 2H).

### Example 307: 5-(((2S,4R)-4-Fluoropyrrolidin-2-yl)methoxy)-N-(1-(3-methoxy

naphthalen-1-yl)cyclopropyl)-2-methylbenzamide (Compound 490)

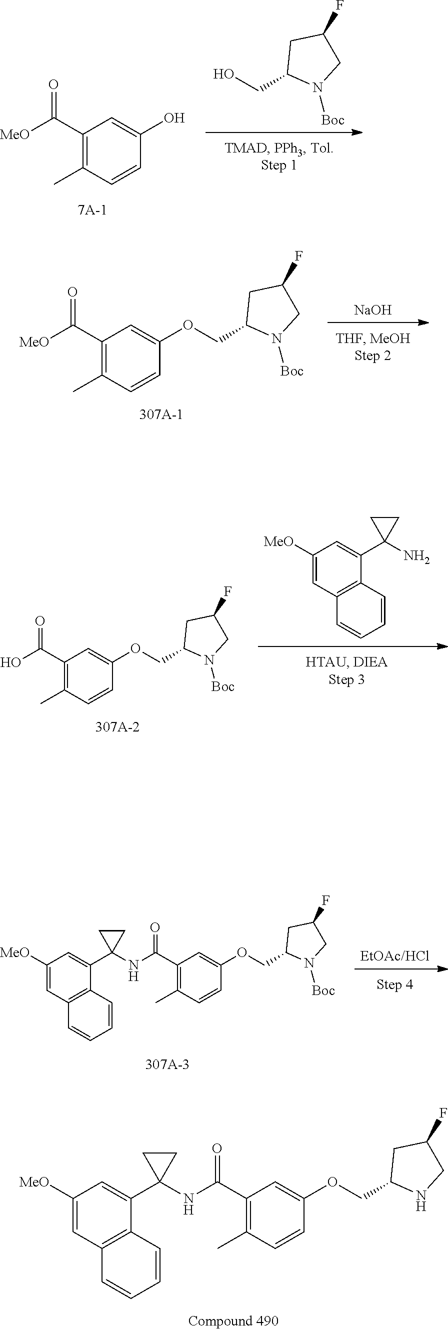

### Step 1: (2S,4R)-tert-Butyl4-fluoro-2-((3-(methoxycarbonyl)-4-methylphenoxy)methyl)pyrrolidine-1-carboxylate (307A-1)

To a solution of methyl 5-hydroxy-2-methylbenzoate (1.17 g, 7.02 mmol, 1.1 eq) and (2S,4R)-tert-butyl 4-fluoro-2-(hydroxymethyl)pyrrolidine-1-carboxylate (1.40 g, 6.39 mmol, 1.0 eq) in toluene (14 mL) were added TMAD (3.30 g, 19.2 mmol, 3.0 eq) and PPh₃ (5.02 g, 19.2 mmol, 3.0 eq). The mixture was degassed and purged with N₂ three times and then was stirred at 100° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/10. (2S,4R)-tert-Butyl 4-fluoro-2-((3-(methoxycarbonyl)-4-methylphenoxy)methyl)pyrrolidine-1-carboxylate (1.20 g, 3.27 mmol, 51% yield) was obtained as a yellow oil. M+H⁺=368.1 (LCMS); 1HNMR (400 MHZ, DMSO-d₆) δ 7.34 (d, J=2.9 Hz, 1H), 7.23 (br d, J=8.4 Hz, 1H), 7.09 (dd, J=2.3, 8.3 Hz, 1H), 5.41-5.18 (m, 1H), 4.22-4.09 (m, 3H), 3.83-3.62 (m, 4H), 3.32 (s, 2H), 2.42 (s, 3H), 1.38 (s, 9H), 1.24-1.14 (m, 1H).

### Step 2: 5-(((2S,4R)-1-(tert-Butoxycarbonyl)-4-fluoropyrrolidin-2-yl)methoxy)-2-methyl benzoic acid (307A-2)

To a solution of (2S,4R)-tert-butyl 4-fluoro-2-((3-(methoxycarbonyl)-4-methylphenoxy)methyl)pyrrolidine-1-carboxylate (1.20 g, 3.27 mmol, 1.0 eq) in a mixture of THF (15 mL) and MeOH (5.0 mL) was added NaOH (2 M in aqueous, 2.0 mL, 1.2 eq). The mixture was stirred at 70° C. for 1 h. LCMS indicated that the starting material was completely consumed, and the desired mass was detected. The reaction mixture was allowed to cool to room temperature, poured into water (10 mL) and extracted with MTBE (5 mL×3). The aqueous was basified to pH 5 by using HCl (1 M, aqueous). The product was extracted with EtOAc (10 mL×3). The combined organic layers were dried over Na₂SO₄, filtered, and concentrated under vacuum to give the crude 5-(((2S,4R)-1-(tert-butoxycarbonyl)-4-fluoropyrrolidin-2-yl)methoxy)-2-methylbenzoic acid (1.10 g, 3.11 mmol, 95% yield) as a yellow oil. M−100+H⁺=254.1 (LCMS).

### Step 3: (2S,4R)-tert-Butyl 4-fluoro-2-((3-((1-(3-methoxynaphthalen-1-yl)cyclopropyl) carbamoyl)-4-methylphenoxy)methyl)pyrrolidine-1-carboxylate (307A-3)

To a solution of 5-(((2S,4R)-1-(tert-butoxycarbonyl)-4-fluoropyrrolidin-2-yl)methoxy)-2-methylbenzoic acid (200 mg, 565 μmol, 1.0 eq) and 1-(3-methoxynaphthalen-1-yl)cyclopropanamine (121 mg, 566 μmol, 1.0 eq) in DMF (2.0 mL) were added DIEA (219 mg, 1.70 mmol, 296 μL, 3.0 eq) and HATU (323 mg, 849 μmol, 1.5 eq). The resulting mixture was stirred at 20° C. for 1 h. LCMS indicated that the starting material was completely consumed, and the desired mass was detected. The mixture was poured into H₂O (10 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/10. (2S,4R)-tert-Butyl 4-fluoro-2-((3-((1-(3-methoxynaphthalen-1-yl)cyclopropyl)carbamoyl)-4-methyl phenoxy)methyl)pyrrolidine-1-carboxylate (200 mg, 364 μmol, 64% yield) was obtained as a yellow oil. M+H⁺=549.1 (LCMS).

### Step 4: 5-(((2S,4R)-4-Fluoropyrrolidin-2-yl)methoxy)-N-(1-(3-methoxynaphthalen-1-yl)cyclopropyl)-2-methylbenzamide (Compound 490)

To a solution of (2S,4R)-tert-butyl 4-fluoro-2-((3-((1-(3-methoxynaphthalen-1-yl)cyclopropyl)carbamoyl)-4-methylphenoxy)methyl)pyrrolidine-1-carboxylate (200 mg, 364 μmol, 1.0 eq) in EtOAc (3.0 mL) was added EtOAc/HCl (4 M, 3.0 mL). The resulting mixture was stirred at 20° C. for 1 h. LCMS indicated that the starting material was consumed, and the desired mass was detected. The mixture was concentrated under vacuum at 20° C. to give a residue which was purified by preparative HPLC (Phenomenex Luna C18 column (80× 30 mm, 3 μm); flow rate: 25 mL/min; gradient: 10%-50% B over 8 min; mobile phase A: 0.04% aqueous HCl, mobile phase B: acetonitrile). 5-(((2S,4R)-4-Fluoropyrrolidin-2-yl)methoxy)-N-(1-(3-methoxynaphthalen-1-yl)cyclopropyl)-2-methylbenzamide (110 mg, 245 μmol, 67% yield, HCl salt) was obtained as a white solid. M+H⁺=449.1 (LCMS); ¹H NMR (400 MHZ, DMSO-d₆) δ 10.12-9.90 (m, 1H), 9.66-9.40 (m, 1H), 9.13 (s, 1H), 8.54 (d, J=8.4 Hz, 1H), 7.83 (d, J=7.9 Hz, 1H), 7.63-7.32 (m, 3H), 7.24 (d, J=2.5 Hz, 1H), 7.08 (d, J=8.5 Hz, 1H), 6.89 (dd, J=2.6, 8.4 Hz, 1H), 6.68 (d, J=2.8 Hz, 1H), 5.57-5.37 (m, 1H), 4.27-4.19 (m, 1H), 4.17-4.09 (m, 1H), 4.08-4.00 (m, 1H), 3.87 (s, 3H), 3.45 (br s, 2H), 2.44-2.30 (m, 1H), 2.07 (s, 1H), 1.98 (s, 3H), 1.34 (br s, 2H), 1.16 (br s, 2H).

### Example 308: 5-(((2S,4R)-4-Fluoro-1-methylpyrrolidin-2-yl)methoxy)-N-(1-(3-methoxy

naphthalen-1-yl)cyclopropyl)-2-methylbenzamide (Compound 489)

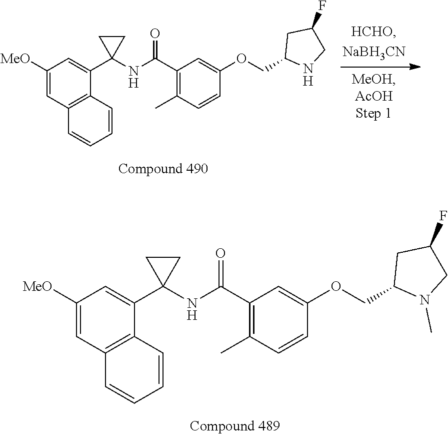 [SMILES: COc1cc(C2(NC(=O)c3cc(OC[C@@H]4C[C@@H](F)CN4)ccc3C)CC2)c2ccccc2c1 ; COc1cc(C2(NC(=O)c3cc(OC[C@@H]4C[C@@H](F)CN4C)ccc3C)CC2)c2ccccc2c1]

### Step 1: 5-(((2S,4R)-4-Fluoro-1-methylpyrrolidin-2-yl)methoxy)-N-(1-(3-methoxy naphthalen-1-yl)cyclopropyl)-2-methylbenzamide (Compound 489)

To a solution of 5-(((2S,4R)-4-fluoropyrrolidin-2-yl)methoxy)-N-(1-(3-methoxynaphthalen-1-yl)cyclopropyl)-2-methylbenzamide (60.0 mg, 133 μmol, 1.0 eq) in MeOH (1.0 mL) was added TEA (20.1 μL), followed by formaldehyde (16.3 mg, 200 μmol, 14.5 μL, 37% purity in water, 1.5 eq). The resulting mixture was adjusted to pH 6 with a small amount of AcOH. The mixture was stirred at 20° C. for 30 min, then NaBH₃CN (50.4 mg, 803 μmol, 6.0 eq) was added. The resulting reaction mixture was stirred at 20° C. for another 16 h. LCMS indicated that the starting material was completely consumed, and the desired mass was detected. The mixture was treated with H₂O (10 mL) and extracted with EtOAc (10 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 20%-50% B over 8 min; mobile phase A: 0.04% aqueous HCl, mobile phase B: acetonitrile). 5-(((2S,4R)-4-Fluoro-1-methylpyrrolidin-2-yl)methoxy)-N-(1-(3-methoxynaphthalen-1-yl)cyclopropyl)-2-methylbenzamide (50.0 mg, 108 μmol, 81% yield, HCl salt) was obtained as a white solid. M+H⁺=463.2 (LCMS); ¹H NMR (400 MHZ, DMSO-d₆) δ 11.1-10.9 (m, 1H), 9.11 (s, 1H), 8.54 (d, J=8.3 Hz, 1H), 7.83 (d, J=7.9 Hz, 1H), 7.49-7.37 (m, 3H), 7.25-7.23 (m, 1H), 7.10-7.07 (m, 1H), 6.93-6.89 (m, 1H), 6.70 (d, J=2.5 Hz, 1H), 5.53-5.36 (m, 1H), 4.35-4.25 (m, 2H), 3.94 (br s, 2H), 3.87 (s, 3H), 3.53-3.42 (m, 1H), 2.96 (br s, 3H), 2.46-2.19 (m, 1H), 2.07 (s, 1H), 1.96 (s, 3H), 1.33 (br s, 2H), 1.19-1.14 (m, 2H).

### Example 309: N-(1-(3-Hydroxynaphthalen-1-yl)cyclopropyl)-2-methyl-5-((1-methylazetidin-2-yl)methoxy)benzamide (Compound 433)

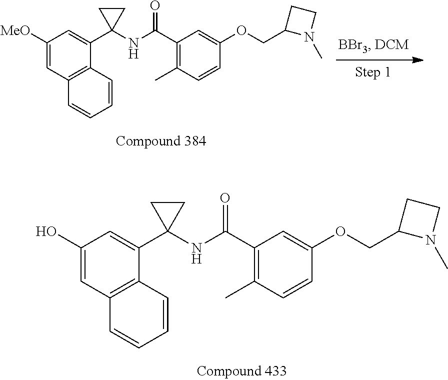 [SMILES: COc1cc(C2(NC(=O)c3cc(OCC4CCN4C)ccc3C)CC2)c2ccccc2c1 ; Cc1ccc(OCC2CCN2C)cc1C(=O)NC1(c2cc(O)cc3ccccc23)CC1]

### Step 1: N-(1-(3-Hydroxynaphthalen-1-yl)cyclopropyl)-2-methyl-5-((1-methylazetidin-2-yl)methoxy)benzamide (Compound 433)

To a mixture of N-(1-(3-methoxynaphthalen-1-yl)cyclopropyl)-2-methyl-5-((1-methylazetidin-2-yl)methoxy)benzamide (70.0 mg, 163 μmol, 1.0 eq) in DCM (5.0 mL) was added dropwise the solution of BBr₃ (326 mg, 1.30 mmol, 125 μL, 8.0 eq) in DCM (500 μL) at −78° C. under a N₂ atmosphere. The mixture was stirred at −78° C. for 1 h then stirred at 20° C. for 2 h. LCMS indicated that the starting material was completely consumed, and the desired mass was detected. The mixture was concentrated under vacuum to give a residue which was diluted with MeOH (3.0 mL) and then acidified to pH 8 by using ammonium hydroxide (25% aqueous). The mixture was concentrated under vacuum to give a residue which was purified by preparative HPLC (Phenomenex Luna C18 column (75×30 mm, 3 μm); flow rate: 25 mL/min; gradient: 20%-60% B over 8 min; mobile phase A: 0.04% aqueous HCl, mobile phase B: acetonitrile). N-(1-(3-Hydroxynaphthalen-1-yl)cyclopropyl)-2-methyl-5-((1-methylazetidin-2-yl)methoxy)benzamide (5.30 mg, 11.2 μmol, 8% yield, HCl salt) was obtained as a white solid. M+H⁺=417.1 (LCMS); ¹H NMR (400 MHZ, CDCl₃) δ 8.52-8.48 (m, 1H), 8.46-8.41 (m, 1H), 7.67 (br d, J=7.6 Hz, 1H), 7.51 (d, J=2.3 Hz, 1H), 7.40-7.31 (m, 2H), 7.09-7.05 (m, 2H), 6.89 (br dd, J=2.5, 8.4 Hz, 1H), 6.70 (d, J=2.3 Hz, 1H), 4.27-4.03 (m, 3H), 3.90-3.81 (m, 1H), 3.65-3.47 (m, 1H), 2.72 (s, 3H), 2.41-2.32 (m, 2H), 2.03 (s, 3H), 1.43-1.28 (m, 4H).

### Example 310: 2-Methyl-5-((1-methylazetidin-2-yl)methoxy)-N-(1-(3-propoxynaphthalen-1-yl)cyclopropyl)benzamide (Compound 474)

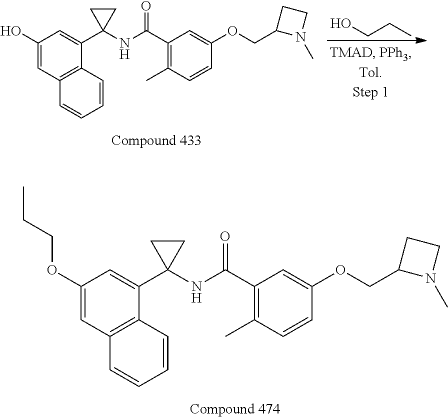

### Step 1: 2-Methyl-5-((1-methylazetidin-2-yl)methoxy)-N-(1-(3-propoxynaphthalen-1-yl)cyclopropyl)benzamide (Compound 474)

A mixture of N-(1-(3-hydroxynaphthalen-1-yl)cyclopropyl)-2-methyl-5-((1-methylazetidin-2-yl)methoxy)benzamide (100 mg, 240 μmol, 1.0 eq), propan-1-ol (15.9 mg, 264 μmol, 19.8 μL, 1.1 eq) and PPh₃ (189 mg, 720 μmol, 3.0 eq) in toluene (5.0 mL) was degassed and purged with N₂ three times. To the mixture was added TMAD (124 mg, 720 μmol, 3.0 eq) at 20° C. The resulting mixture was stirred at 100° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×4). The combined organic layers were dried over Na₂SO₄, filtered, and concentrated under vacuum to give a residue which was purified by preparative HPLC (Phenomenex Luna C18 column (200×40 mm, 10 μm); flow rate: 75 mL/min; gradient: 20%-50% B over 8 min; mobile phase A: 0.2% aqueous FA, mobile phase B: acetonitrile). 2-Methyl-5-((1-methylazetidin-2-yl)methoxy)-N-(1-(3-propoxynaphthalen-1-yl)cyclopropyl)benzamide (14.9 mg, 32.0 μmol, 13% yield, FA salt) was obtained as a white solid. M+H⁺=459.2 (LCMS); ¹H NMR (400 MHZ, DMSO-d₆) δ 9.17-8.98 (m, 1H), 8.57-8.49 (m, 1H), 8.20-8.17 (m, 1H), 7.84-7.78 (m, 1H), 7.48-7.34 (m, 3H), 7.24-7.20 (m, 1H), 7.05-6.99 (m, 1H), 6.88-6.80 (m, 1H), 6.61-6.57 (m, 1H), 4.06-4.00 (m, 2H), 3.89-3.84 (m, 2H), 3.30-3.24 (m, 2H), 2.81-2.70 (m, 1H), 2.25-2.21 (m, 3H), 2.00-1.77 (m, 7H), 1.36-1.30 (m, 2H), 1.19-1.13 (m, 2H), 1.07-1.00 (m, 3H).

### Example 311: N-(1-(3-Bromonaphthalen-1-yl)cyclopropyl)-2-methyl-5-((1-methylazetidin-2-yl)methoxy)benzamide (Compound 461)

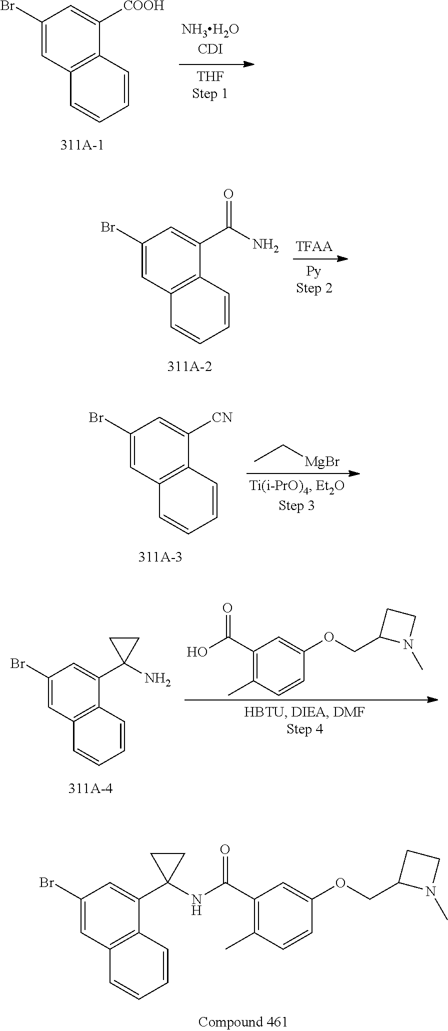

### Step 1:3-Bromo-1-naphthamide (311A-2)

To a solution of 3-bromo-1-naphthoic acid (3.00 g, 12.0 mmol, 1.0 eq) in THF (30 mL) was added CDI (2.90 g, 17.9 mmol, 1.5 eq) at 0° C. The mixture was stirred at 20° C. for 1.5 h. Then NH₃·H₂O (9.10 g, 64.9 mmol, 10.0 mL, 25% purity, 5.4 eq) was added to the mixture, the resulting mixture was stirred at 20° C. for 1 h. LCMS indicated that the starting material was completely consumed, and the desired mass was detected. The mixture was concentrated under vacuum to give a residue. The residue was triturated in H₂O (15 mL) at 20° C. for 15 min. The mixture was filtered and the filter cake was washed subsequently by H₂O (8.0 mL×2) and HCl (1 M aqueous) (10 mL×1). The filter cake was then concentrated under vacuum to give a crude product 3-bromo-1-naphthamide (2.92 g) as a white solid. M+H⁺=250.1 (LCMS).

### Step 2:3-Bromo-1-naphthonitrile (311A-3)

To a solution of 3-bromo-1-naphthamide (240 mg, 960 μmol, 1.0 eq) in DMF (5.0 mL) were added TEA (400 mg, 3.95 mmol, 550 μL, 4.1 eq) and TFAA (410 mg, 1.95 mmol, 271 μL, 2.0 eq) at 0° C. The mixture was stirred at 20° C. for 6 h. LCMS indicated that the starting material was completely consumed. The mixture was concentrated under vacuum at 30° C. to give a residue which was purified by flash silica gel chromatography using a gradient of EtOAc/petroleum ether from 0/1 to 8/100 to give 3-bromo-1-naphthonitrile (188 mg, 810 μmol, 84% yield) as a white solid. ¹H NMR (400 MHZ, CDCl₃) δ 8.38-8.14 (m, 2H), 8.07-7.94 (m, 1H), 7.93-7.81 (m, 1H), 7.79-7.60 (m, 2H).

### Step 3: 1-(3-Bomonaphthalen-1-yl)cyclopropanamine (311A-4)

A mixture of 3-bromo-1-naphthonitrile (180 mg, 776 mmol, 1.0 eq) in anhydrous Et₂O (10 mL) was degassed and purged with N₂ three times. The mixture was stirred at −78° C. To this mixture was added Ti(i-PrO)₄ (331 mg, 1.16 mmol, 343 μL, 1.5 eq) slowly, and then EtMgBr (3 M, 595 μL, 2.3 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. BF₃·Et₂O (220 mg, 1.55 mmol, 191 μL, 2.0 eq) was added slowly with no obvious temperature change. The mixture was stirred at 20° C. for another 1 h. LCMS indicated that the starting material was completely consumed, and desired mass was detected. The reaction mixture was poured into HCl (1 M aqueous) (7.0 mL), and the mixture was extracted with EtOAc (5.0 mL×3). The organic phase was discarded. The aqueous layer was basified to pH 8 by using saturated Na₂CO₃ aqueous solution and a precipitate was formed. The mixture was filtered through a pad of Celite and the slurry was washed with EtOAc several times. The combined filtrate was extracted with EtOAc (8.0 mL×4). The combined organic layers were dried over Na₂SO₄, filtered, and concentrated under vacuum to give a crude product 1-(3-bomonaphthalen-1-yl)cyclopropanamine (80.0 mg) as a yellow oil. M+H⁺=262.4 (LCMS).

### Step 4: N-(1-(3-Bromonaphthalen-1-yl)cyclopropyl)-2-methyl-5-((1-methylazetidin-2-yl)methoxy)benzamide (Compound 461)

To a solution of 1-(3-bromonaphthalen-1-yl)cyclopropanamine (111 mg, 425 μmol, 1.0 eq) in DMF (2.0 mL) was added 2-methyl-5-((1-methylazetidin-2-yl)methoxy)benzoic acid (100 mg, 425 μmol, 1.0 eq), followed by HBTU (322 mg, 850 μmol, 2.0 eq) and DIEA (164 mg, 1.28 mmol, 222 μL, 3.0 eq). The mixture was stirred at 20° C. for 16 h. LCMS indicated that 8% starting material remained and 37% desired compound was detected. The mixture was filtered and the filtrate was purified by preparative HPLC (Phenomenex Luna column (80×30 mm, 3 μm); flow rate: 25 mL/min; gradient: 25%-55% B over 8 min; mobile phase A: 0.1% aqueous TFA, mobile phase B: acetonitrile) to give N-(1-(3-bromonaphthalen-1-yl)cyclopropyl)-2-methyl-5-((1-methylazetidin-2-yl)methoxy)benzamide (95.0 mg, 156 μmol, 38% yield, TFA salt) as a yellow solid. M+H⁺=479.1 (LCMS); ¹H NMR (400 MHZ, CDCl₃) δ ppm 13.35-12.45 (m, 1H), 8.67-8.43 (m, 1H), 8.11-8.02 (m, 1H), 8.00-7.92 (m, 1H), 7.83-7.75 (m, 1H), 7.61-7.48 (m, 2H), 7.40 (br s, 1H), 7.09-6.90 (m, 1H), 6.81-6.53 (m, 2H), 4.36-4.31 (m, 2H), 4.27-4.08 (m, 1H), 3.97-3.83 (m, 1H), 3.75-3.57 (m, 1H), 2.94-2.71 (m, 3H), 2.63-2.38 (m, 2H), 2.15 (s, 3H), 1.63-1.51 (m, 2H), 1.45-1.29 (m, 2H).

### Example 312: (S)-5-(2-Aminopropoxy)-N-(1-(3-bromonaphthalen-1-yl)cyclopropyl)-2-methylbenzamide (Compound 541)

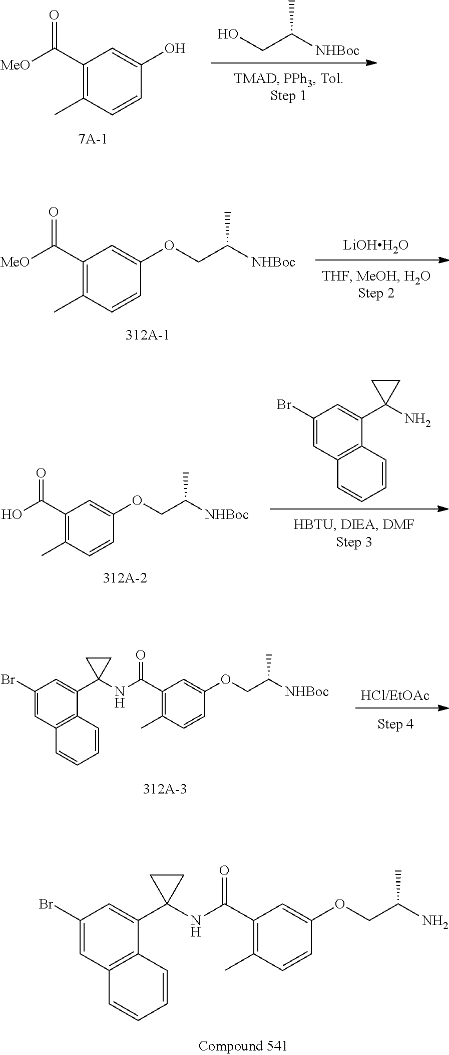

### Step 1: (S)-Methyl 5-(2-((tert-butoxycarbonyl)amino)propoxy)-2-methylbenzoate (312A-1)

To a solution of methyl 5-hydroxy-2-methylbenzoate (10.5 g, 60.2 mmol, 1.0 eq) in toluene (200 mL) was added(S)-tert-butyl(1-hydroxypropan-2-yl)carbamate (1.00 g, 6.02 mmol, 1.0 eq), followed by TMAD (31.1 g, 180 mmol, 3.0 eq) and PPh₃ (47.4 g, 181 mmol, 3.0 eq). The mixture was stirred at 100° C. for 16 h under a N₂ atmosphere. LCMS indicated that the starting material remained, and the desired mass was detected. The mixture was allowed to cool to room temperature, poured into water (30 mL), and then extracted with DCM (20 mL×3). The combined organic layers were dried over Na₂SO₄ filtered, and concentrated under vacuum to give a residue which was purified by flash silica gel chromatography using a gradient of EtOAc/petroleum ether from 0/1 to 1/20. (S)-Methyl 5-(2-((tert-butoxycarbonyl)amino)propoxy)-2-methylbenzoate (4.30 g, 12.5 mmol, 21% yield) was obtained as a white solid. M−100+H⁺=224.1 (LCMS).

### Step 2: (S)-5-(2-((tert-Butoxycarbonyl)amino)propoxy)-2-methylbenzoic acid (312A-2)

To a solution of(S)-methyl 5-(2-((tert-butoxycarbonyl)amino)propoxy)-2-methylbenzoate (3.50 g, 10.8 mmol, 1.0 eq) in a mixture of THF (16 mL), MeOH (12 mL) and H₂O (8.0 mL) was added LiOH·H₂O (1.36 g, 32.5 mmol, 3.0 eq). The mixture was stirred at 20° C. for 16 h under a N₂ atmosphere. LCMS indicated that the starting material was completely consumed, and the desired mass was detected. The reaction mixture was poured into H₂O (10 mL) and extracted with EtOAc (10 mL×2). The organic phase was discarded. The aqueous layer was acidified to pH 6 by using HCl (1 M aqueous) and then extracted with EtOAc (10 mL×3). The combined organic layers were dried over Na₂SO₄ filtered, and concentrated under vacuum to give a crude product(S)-5-(2-((tert-butoxycarbonyl)amino)propoxy)-2-methylbenzoic acid (3.10 g) as a white solid. M−100+H⁺=210.2 (LCMS).

### Step 3: (S)-tert-Butyl(1-(3-((1-(3-bromonaphthalen-1-yl)cyclopropyl)carbamoyl)-4-methylphenoxy)propan-2-yl)carbamate (312A-3)

To a solution of 1-(3-bromonaphthalen-1-yl)cyclopropanamine (150 mg, 572 μmol, 1.0 eq) in DMF (2.0 mL) was added(S)-5-(2-((tert-butoxycarbonyl)amino)propoxy)-2-methylbenzoic acid (212 mg, 686 μmol, 1.2 eq), followed by HBTU (434 mg, 1.14 mmol, 2.0 eq) and DIEA (221 mg, 1.72 mmol, 299 μL, 3.0 eq). The mixture was stirred at 20° C. for 16 h. LCMS indicated that the starting material was completely consumed. The mixture was treated with water (10 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 13/100. (S)-tert-Butyl(1-(3-((1-(3-bromonaphthalen-1-yl)cyclopropyl)carbamoyl)-4-methylphenoxy)propan-2-yl)carbamate (420 mg, 759 μmol, 79% yield) was obtained as a yellow oil. M−56+H⁺=497.1 (LCMS).

### Step 4: (S)-5-(2-Aminopropoxy)-N-(1-(3-bromonaphthalen-1-yl)cyclopropyl)-2-methylbenzamide (Compound 541)

To a solution of HCl/EtOAc (4 M, 4.0 mL) was added(S)-tert-butyl(1-(3-((1-(3-bromonaphthalen-1-yl)cyclopropyl)carbamoyl)-4-methylphenoxy)propan-2-yl)carbamate (360 mg, 650 μmol, 1.0 eq). The mixture was stirred at 20° C. for 2 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 10%-40% B over 8 min; mobile phase A: 0.04% aqueous HCl, mobile phase B: acetonitrile). (S)-5-(2-Aminopropoxy)-N-(1-(3-bromonaphthalen-1-yl)cyclopropyl)-2-methylbenzamide (161 mg, 328 μmol, HCl salt) was obtained as a white solid. M+H⁺=453.1 (LCMS); ¹H NMR (400 MHZ, DMSO-d₆) δ 9.21 (s, 1H), 8.68-8.59 (m, 1H), 8.15 (s, 1H), 8.09 (br s, 2H), 7.94 (d, J=7.89 Hz, 1H), 7.88 (d, J=1.97 Hz, 1H), 7.66-7.54 (m, 2H), 7.08 (d, J=8.33 Hz, 1H), 6.89 (dd, J=8.33, 2.63 Hz, 1H), 6.68 (d, J=2.63 Hz, 1H), 4.03 (dd, J=10.41, 3.84 Hz, 1H), 3.88 (dd, J=10.19, 6.91 Hz, 1H), 3.58-3.45 (m, 1H), 1.96 (s, 3H), 1.35 (br s, 2H), 1.23 (d, J=6.58 Hz, 5H).

### Example 313: N-(1-(3-Cyanonaphthalen-1-yl)cyclopropyl)-5-(2-(dimethylamino)ethoxy)-2-methylbenzamide (Compound 443)

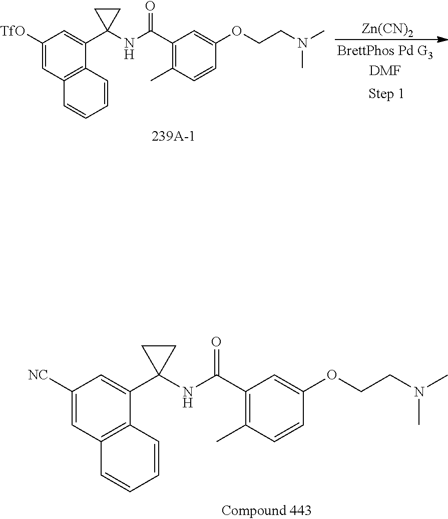

### Step 1: N-(1-(3-Cyanonaphthalen-1-yl)cyclopropyl)-5-(2-(dimethylamino)ethoxy)-2-methylbenzamide (Compound 443)

To a solution of 4-(1-(5-(2-(dimethylamino)ethoxy)-2-methylbenzamido)cyclopropyl) naphthalen-2-yl trifluoromethanesulfonate (65.0 mg, 121 μmol, 1.0 eq) in DMF (3.0 mL) were added Zn(CN)₂ (28.5 mg, 242 μmol, 15.4 μL, 2.0 eq), BrettPhos (11.0 mg, 12.1 μmol, 0.10 eq) and BrettPhos Pd G3 (13.0 mg, 24.2 μmol, 0.20 eq). The mixture was degassed and purged with N₂ three times. The resulting mixture was stirred at 80° C. for 16 h under a N₂ atmosphere. LCMS indicated that the starting material was completely consumed, and the desired mass was detected. The reaction mixture was allowed to cool to room temperature, poured into H₂O (5.0 mL) and extracted with EtOAc (3.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 20%-55% B over 8 min; mobile phase A: 0.2% aqueous FA, mobile phase B: acetonitrile). N-(1-(3-Cyanonaphthalen-1-yl)cyclopropyl)-5-(2-(dimethylamino)ethoxy)-2-methylbenzamide (8.50 mg, 20.6 μmol, 17% yield) was obtained as a white solid. M+H⁺=414.1 (LCMS); ¹H NMR (400 MHZ, DMSO-d₆) δ 9.19 (s, 1H), 8.72 (d, J=8.5 Hz, 1H), 8.52 (s, 1H), 8.26 (s, 1H), 8.10 (d, J=8.0 Hz, 1H), 7.98 (d, J=1.5 Hz, 1H), 7.85-7.76 (m, 1H), 7.73-7.65 (m, 1H), 7.03 (d, J=8.5 Hz, 1H), 6.84 (dd, J=2.8, 8.4 Hz, 1H), 6.61 (d, J=2.6 Hz, 1H), 3.94 (t, J=5.8 Hz, 2H), 2.55 (t, J=5.8 Hz, 2H), 2.17 (s, 6H), 1.93 (s, 3H), 1.37 (br s, 2H), 1.26 (br s, 2H).

### Example 314: N-(1-(3-Cyanonaphthalen-1-yl)cyclopropyl)-2-methyl-5-((1-methylazetidin-2-yl)methoxy)benzamide (Compound 484)

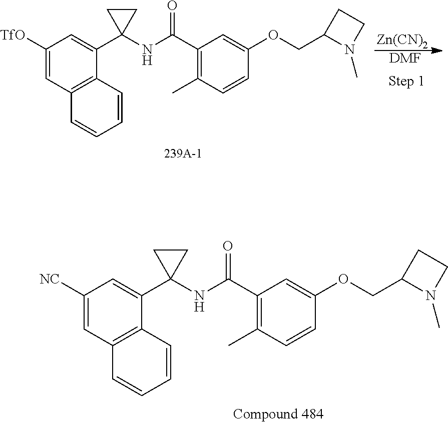

### Step 1: N-(1-(3-Cyanonaphthalen-1-yl)cyclopropyl)-2-methyl-5-((1-methylazetidin-2-yl)methoxy)benzamide (Compound 484)

To a solution of 4-(1-(2-methyl-5-((1-methylazetidin-2-yl)methoxy)benzamido)cyclopropyl) naphthalen-2-yl trifluoromethanesulfonate (100 mg, 182 μmol, 1.0 eq) in DMF (4.0 mL) were added ZnCN₂ (64.2 mg, 547 μmol, 34.7 μL, 3.0 eq), BrettPhos (19.6 mg, 36.5 μmol, 0.2 eq) and BrettPhos Pd G3 (16.5 mg, 18.2 μmol, 0.1 eq) under a N₂ atmosphere. The mixture was degassed and purged with N₂ three times. The resulting mixture was stirred at 80° C. for 16 h. LCMS indicated that the starting material was completely consumed, and the desired mass was detected. The reaction mixture was allowed to cool to room temperature, poured into H₂O (4.0 mL) and extracted with EtOAc (4.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 30%-60% B over 8 min; mobile phase A: 0.2% aqueous FA, mobile phase B:

acetonitrile). N-(1-(3-Cyanonaphthalen-1-yl)cyclopropyl)-2-methyl-5-((1-methylazetidin-2-yl)methoxy)benzamide (20.5 mg, 42.9 μmol, 24% yield, FA salt) was obtained as a white solid. M+H⁺=426.1 (LCMS); ¹H NMR (400 MHZ, DMSO-d₆)) δ 9.19 (s, 1H), 8.73 (d, J=8.5 Hz, 1H), 8.53 (s, 1H), 8.11 (d, J=8.0 Hz, 1H), 7.99 (d, J=1.4 Hz, 1H), 7.88-7.75 (m, 1H), 7.74-7.60 (m, 1H), 7.06 (d, J=8.5 Hz, 1H), 6.87 (dd, J=2.6, 8.3 Hz, 1H), 6.65 (d, J=2.5 Hz, 1H), 3.99 (br d, J=5.3 Hz, 2H), 3.68 (br s, 2H), 3.10 (br s, 1H), 2.42 (s, 3H), 2.01 (s, 2H), 1.94 (s, 3H), 1.38 (br s, 2H), 1.28 (br s, 2H).

### Example 315: tert-Butyl(4-(1-(2-methyl-5-((1-methylazetidin-2-yl)methoxy)benzamido)cyclopropyl)naphthalen-2-yl)carbamate (Compound 482)

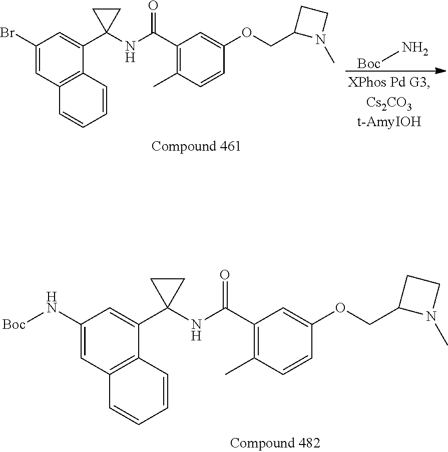

### Step 1: tert-Butyl(4-(1-(2-methyl-5-((1-methylazetidin-2-yl)methoxy)benzamido)cyclopropyl)naphthalen-2-yl)carbamate (Compound 482)

To a solution of N-(1-(3-bromonaphthalen-1-yl)cyclopropyl)-2-methyl-5-((1-methylazetidin-2-yl)methoxy)benzamide (100 mg, 209 μmol, 1.0 eq) and tert-butyl carbamate (29.3 mg, 250 μmol, 1.2 eq) in t-AmylOH (1.0 mL) were added Xphos Pd G3 (35.3 mg, 41.7 μmol, 0.2 eq) and Cs₂CO₃ (272 mg, 834 μmol, 4.0 eq). The mixture was stirred at 80° C. under a N₂ atmosphere for 16 h. LCMS indicated that the starting material was completely consumed. The reaction mixture was allowed to cool to room temperature, concentrated under vacuum to give a residue which was purified by preparative HPLC (Phenomenex Luna C18 column (75× 30 mm, 3 μm); flow rate: 25 mL/min; gradient: 30%-60% B over 8 min; mobile phase A: 0.1% aqueous TFA, mobile phase B: acetonitrile). tert-Butyl(4-(1-(2-methyl-5-((1-methylazetidin-2-yl)methoxy)benzamido)cyclopropyl)naphthalen-2-yl)carbamate (13.0 mg, 20.7 μmol, 10% yield, TFA salt) was obtained as a white solid. M+H⁺=516.3 (LCMS); ¹H NMR (400 MHZ, CD₃OD) δ 9.20 (s, 1H), 8.51-8.42 (m, 1H), 7.99 (br s, 1H), 7.83-7.74 (m, 2H), 7.48-7.38 (m, 2H), 7.10 (d, J=8.50 Hz, 1H), 6.93 (dd, J=8.38, 2.75 Hz, 1H), 6.71 (d, J=2.75 Hz, 1H), 4.70-4.60 (m, 1H), 4.29-4.21 (m, 1H), 4.20-4.10 (m, 2H), 3.94 (q, J=9.55 Hz, 1H), 2.92 (s, 3H), 2.60-2.47 (m, 2H), 2.04 (s, 3H), 1.56 (s, 9H), 1.46-1.40 (m, 2H), 1.36-1.28 (m, 2H).

### Example 316: N-(1-(3-Aminonaphthalen-1-yl)cyclopropyl)-2-methyl-5-((1-methylazetidin-2-yl)methoxy)benzamide (Compound 496)

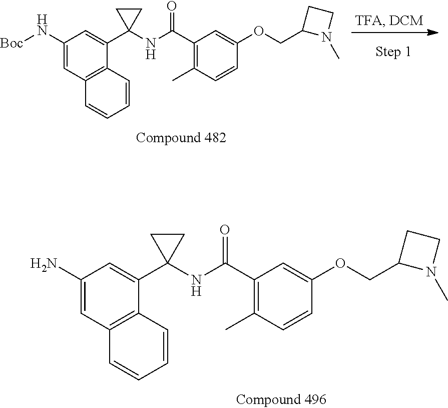 [SMILES: Cc1ccc(OCC2CCN2C)cc1C(=O)NC1(c2cc(NC(=O)OC(C)(C)C)cc3ccccc23)CC1 ; Cc1ccc(OCC2CCN2C)cc1C(=O)NC1(c2cc(N)cc3ccccc23)CC1]

### Step 1: N-(1-(3-Aminonaphthalen-1-yl)cyclopropyl)-2-methyl-5-((1-methylazetidin-2-yl) methoxy)benzamide (Compound 496)

To a solution of tert-butyl(4-(1-(2-methyl-5-((1-methylazetidin-2-yl)methoxy)benzamido)cyclopropyl)naphthalen-2-yl)carbamate (170 mg, 330 μmol, 1.0 eq) in DCM (2.0 mL) was added TFA (2.0 mL). The mixture was stirred at 20° C. for 30 min. LCMS indicated that the starting material was completely consumed. The reaction mixture was concentrated under vacuum to give a residue which was purified by preparative HPLC (Phenomenex Luna C18 column (75×30 mm, 3 μm); flow rate: 25 mL/min; gradient: 1%-25% B over 8 min; mobile phase A: 0.1% aqueous TFA, mobile phase B: acetonitrile) to give N-(1-(3-aminonaphthalen-1-yl)cyclopropyl)-2-methyl-5-((1-methylazetidin-2-yl)methoxy)benzamide (36.8 mg, 68.9 μmol, 21% yield, TFA salt) as a pale yellow solid. M+H⁺=416.1 (LCMS); ¹H NMR (400 MHZ, CD₃OD) δ 9.38-9.28 (m, 1H), 8.61-8.54 (m, 1H), 7.95-7.90 (m, 1H), 7.80 (d, J=2.3 Hz, 1H), 7.73-7.66 (m, 1H), 7.65-7.56 (m, 2H), 7.10 (d, J=8.5 Hz, 1H), 6.98-6.92 (m, 1H), 6.76 (d, J=2.6 Hz, 1H), 4.73-4.62 (m, 1H), 4.30-4.14 (m, 3H), 4.02-3.89 (m, 1H), 3.00-2.90 (m, 3H), 2.60-2.50 (m, 2H), 1.99-1.96 (m, 3H), 1.51-1.46 (m, 2H), 1.37-1.32 (m, 2H).

### Example 317: N-(1-(3-(Dimethylamino) naphthalen-1-yl)cyclopropyl)-2-methyl-5-((1-methylazetidin-2-yl)methoxy)benzamide (Compound 495)

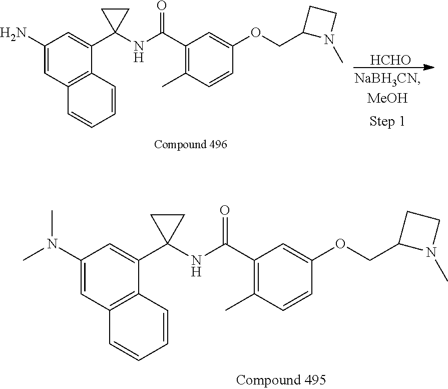

### Step 1: N-(1-(3-(Dimethylamino) naphthalen-1-yl)cyclopropyl)-2-methyl-5-((1-methylazetidin-2-yl)methoxy)benzamide (Compound 495)

To a solution of N-(1-(3-aminonaphthalen-1-yl)cyclopropyl)-2-methyl-5-((1-methylazetidin-2-yl)methoxy)benzamide (60.0 mg, 144 μmol, 1.0 eq, TFA salt) in MeOH (2.0 mL) was added TEA (20 μL), followed by formaldehyde (25.8 mg, 318 μmol, 23.6 μL, 37% purity in water, 2.2 eq). The resulting mixture was adjusted to pH 6 with a small amount of AcOH. The mixture was stirred at 20° C. for 30 min, then NaBH₃CN (22.7 mg, 361 μmol, 2.5 eq) was added. The resulting mixture was stirred at 20° C. for another 16 h. LCMS indicated that the starting material was completely consumed. The reaction mixture was concentrated under vacuum to give a residue which was purified by preparative HPLC (Phenomenex Luna C18 column (75×30 mm, 3 μm); flow rate: 25 mL/min; gradient: 1%-25% B over 8 min; mobile phase A: 0.1% aqueous TFA, mobile phase B: acetonitrile). N-(1-(3-(Dimethylamino) naphthalen-1-yl)cyclopropyl)-2-methyl-5-((1-methyl azetidin-2-yl)methoxy)benzamide (23.1 mg, 41.1 μmol, 28% yield, TFA salt) was obtained as a yellow gum. M+H⁺=444.2 (LCMS); ¹H NMR (400 MHz, CD₃OD) δ9.33-9.23 (m, 1H), 8.50 (br d, J=7.8 Hz, 1H), 7.93-7.82 (m, 2H), 7.61-7.41 (m, 3H), 7.11 (d, J=8.4 Hz, 1H), 7.01-6.90 (m, 1H), 6.74 (d, J=2.6 Hz, 1H), 4.75-4.60 (m, 1H), 4.30-4.24 (m, 1H), 4.24-4.11 (m, 2H), 4.00-3.91 (m, 1H), 3.26-3.24 (m, 6H), 3.03-2.88 (m, 3H), 2.59-2.50 (m, 2H), 2.02-1.98 (m, 3H), 1.51-1.44 (m, 2H), 1.39-1.32 (m, 2H).

### Example 318:4-(1-(2-Methyl-5-((1-methylazetidin-2-yl)methoxy)benzamido)cyclopropyl)-2-naphthamide (Compound 520)

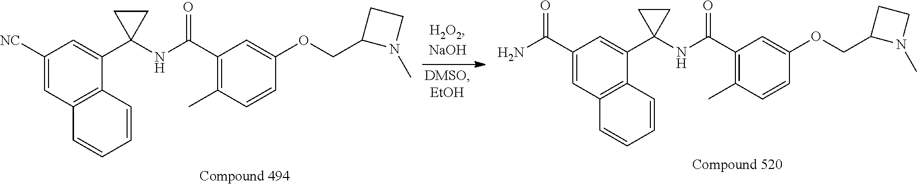 [SMILES: Cc1ccc(OCC2CCN2C)cc1C(=O)NC1(c2cc(C#N)cc3ccccc23)CC1 ; Cc1ccc(OCC2CCN2C)cc1C(=O)NC1(c2cc(C(N)=O)cc3ccccc23)CC1]

### Step 1:4-(1-(2-Methyl-5-((1-methylazetidin-2-yl)methoxy)benzamido)cyclopropyl)-2-naphthamide (Compound 520)

To a solution of N-(1-(3-cyanonaphthalen-1-yl)cyclopropyl)-2-methyl-5-((1-methylazetidin-2-yl)methoxy)benzamide (30.0 mg, 70.5 μmol, 1.0 eq) in a mixture of DMSO (0.3 mL) and EtOH (0.9 mL) were added H₂O₂ (35.9 mg, 212 μmol, 30.5 μL, 20% purity in H₂O, 3.0 eq) and NaOH (8.46 mg, 212 μmol, 3.0 eq). The mixture was stirred at 20° C. for 1 h. LCMS indicated that the starting material was completely consumed, and the desired mass was detected. The mixture was poured into H₂O (1.0 mL) and extracted with EtOAc (0.5 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 25%-45% B over 8 min; mobile phase A: 0.2% aqueous FA, mobile phase B: acetonitrile). 4-(1-(2-Methyl-5-((1-methylazetidin-2-yl)methoxy)benzamido)cyclopropyl)-2-naphthamide (6.30 mg, 13.8 μmol, 20% yield, FA salt) was obtained as a white solid. M+H⁺=444.1 (LCMS); ¹H NMR (400 MHZ, DMSO-d₆)) δ 9.13 (s, 1H), 8.68 (d, J=8.1 Hz, 1H), 8.39 (s, 1H), 8.23 (s, 1H), 8.19-8.08 (m, 1H), 8.02 (d, J=8.6 Hz, 1H), 7.78-7.62 (m, 1H), 7.59 (br d, J=7.1 Hz, 1H), 7.48 (s, 1H), 7.07 (d, J=8.3 Hz, 1H), 6.95-6.82 (m, 1H), 6.68 (d, J=1.7 Hz, 1H), 4.25-4.05 (m, 2H), 3.92-3.70 (m, 1H), 2.72-2.62 (m, 2H), 2.49-2.40 (m, 3H), 2.31-2.08 (m, 2H), 1.95 (s, 3H), 1.37 (br s, 2H), 1.26 (br s, 2H).

### Example 319: 2-Methyl-5-((1-methylazetidin-2-yl)methoxy)-N-(1-(3-(thiophen-2-yl)naphthalen-1-yl)cyclopropyl)benzamide (Compound 481)

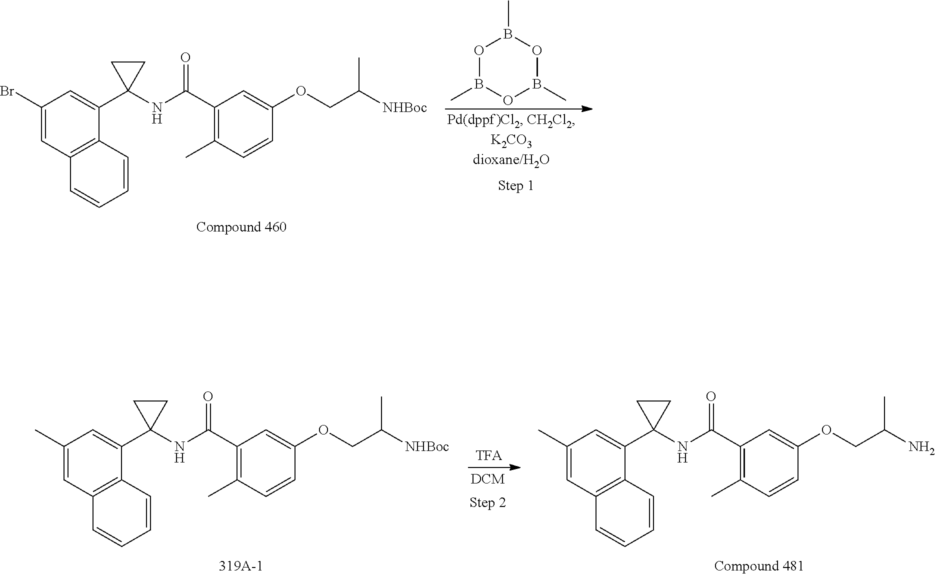

### Step 1: tert-Butyl(1-(4-methyl-3-((1-(3-methylnaphthalen-1-yl)cyclopropyl)carbamoyl)phenoxy)propan-2-yl)carbamate (319A-1)

To a solution of tert-butyl(1-(3-((1-(3-bromonaphthalen-1-yl)cyclopropyl)carbamoyl)-4-methylphenoxy)propan-2-yl)carbamate (80.0 mg, 145 μmol, 1.0 eq) in DMF (2.0 mL) were added 2,4,6-trimethyl-1,3,5,2,4,6-trioxatriborinane (272 mg, 1.08 mmol, 303 μL, 50% purity, 7.5 eq), Pd(dppf)Cl₂·CH₂Cl₂ (11.8 mg, 14.5 μmol, 0.1 eq) and Cs₂CO₃ (155 mg, 477 μmol, 3.3 eq). The mixture was stirred at 110° C. for 3 h under a N₂ atmosphere. LCMS indicated that the starting material was completely consumed. The reaction mixture was allowed to cool to room temperature, poured into water (10 mL) and extracted with EtOAc (8.0 mL×3). The combined organic layers were dried over Na₂SO₄, filtered, and concentrated under vacuum to give a residue which was purified by preparative TLC (EtOAc/petroleum ether=1/1, R_{f}=0.5). tert-Butyl(1-(4-methyl-3-((1-(3-methylnaphthalen-1-yl)cyclopropyl)carbamoyl)phenoxy)propan-2-yl)carbamate (90.0 mg, 120 μmol, 82% yield) was obtained as a yellow solid. M+H⁺=289.4 (LCMS).

### Step 2: 5-(2-Aminopropoxy)-2-methyl-N-(1-(3-methylnaphthalen-1-yl)cyclopropyl)benzamide (Compound 481)

To a solution of tert-butyl(1-(4-methyl-3-((1-(3-methylnaphthalen-1-yl)cyclopropyl)carbamoyl)phenoxy)propan-2-yl)carbamate (90.0 mg, 184 μmol, 1.0 eq) in DCM (2.0 mL) was added TFA (2.0 mL). The mixture was stirred at 20° C. for 1 h. LCMS indicated that the starting material was completely consumed. The reaction mixture was concentrated under vacuum to give a residue which was purified by preparative HPLC (Phenomenex Luna C18 column (150×30 mm, 5 μm); flow rate: 25 mL/min; gradient: 15%-45% B over 8 min; mobile phase A: 0.1% aqueous TFA, mobile phase B: acetonitrile) to give 5-(2-aminopropoxy)-2-methyl-N-(1-(3-methylnaphthalen-1-yl)cyclopropyl)benzamide (20.4 mg, 40.6 μmol, 22% yield, TFA salt) as a yellow solid. M+H⁺=389.2 (LCMS); ¹H NMR (400 MHz, DMSO-d₆) δ 9.09 (s, 1H), 8.62-8.55 (m, 1H), 7.92 (br s, 2H), 7.79-7.86 (m, 1H), 7.66 (d, J=1.63 Hz, 1H), 7.60 (s, 1H), 7.51-7.43 (m, 2H), 7.08 (d, J=8.50 Hz, 1H), 6.88 (dd, J=8.32, 2.69 Hz, 1H), 6.66 (d, J=2.75 Hz, 1H), 4.02 (dd, J=10.26, 3.75 Hz, 1H), 3.84 (dd, J=10.32, 7.19 Hz, 1H), 3.59-3.45 (m, 1H), 2.47 (s, 3H), 1.99 (s, 3H), 1.33 (br s, 2H), 1.21 (d, J=6.63 Hz, 3H), 1.17 (br s, 2H).

### Example 320: 2-Methyl-5-((1-methylazetidin-2-yl)methoxy)-N-(1-(3-methylnaphthalen-1-yl)cyclopropyl)benzamide (Compound 501)

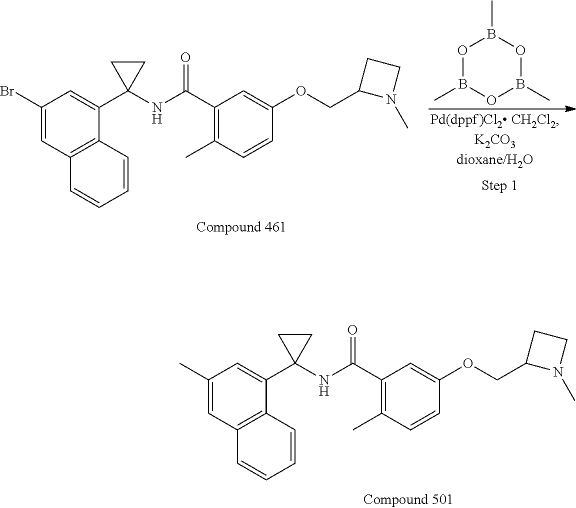

### Step 1: 2-Methyl-5-((1-methylazetidin-2-yl)methoxy)-N-(1-(3-methylnaphthalen-1-yl)cyclopropyl)benzamide (Compound 501)

To a solution of N-(1-(3-bromonaphthalen-1-yl)cyclopropyl)-2-methyl-5-((1-methylazetidin-2-yl)methoxy)benzamide (80.0 mg, 167 μmol, 1.0 eq), 2,4,6-trimethyl-1,3,5,2,4,6-trioxatriborinane (83.8 mg, 334 μmol, 93.3 μL, 50% purity, 2.0 eq) and Cs₂CO₃ (179 mg, 551 μmol, 3.3 eq) in DMF (1.0 mL) was added Pd(dppf)Cl₂·CH₂Cl₂ (13.6 mg, 16.7 μmol, 0.1 eq). The mixture was stirred at 110° C. for 1 h under a N₂ atmosphere. LCMS indicated that the starting material was completely consumed. The reaction mixture was allowed to cool to room temperature and filtered. The filtrate was purified by preparative HPLC (Phenomenex Luna C18 column (75×30 mm, 3 μm); flow rate: 25 mL/min; gradient: 5%-40% B over 8 min; mobile phase A: 0.1% aqueous TFA, mobile phase B: acetonitrile). 2-Methyl-5-((1-methylazetidin-2-yl)methoxy)-N-(1-(3-methylnaphthalen-1-yl)cyclopropyl)benzamide (35.0 mg, 66.1 μmol, 39% yield, TFA salt) was obtained as a pale yellow solid. M+H⁺=415.2 (LCMS); ¹H NMR (400 MHZ, DMSO-d₆) δ 9.90 (br s, 1H), 9.09 (s, 1H), 8.62-8.54 (m, 1H), 7.87-7.78 (m, 1H), 7.66 (d, J=1.50 Hz, 1H), 7.61 (s, 1H), 7.53-7.42 (m, 2H), 7.09 (d, J=8.50 Hz, 1H), 6.90 (dd, J=8.38, 2.75 Hz, 1H), 6.69 (d, J=2.63 Hz, 1H), 4.60 (br d, J=4.38 Hz, 1H), 4.28-4.15 (m, 2H), 4.08-3.95 (m, 1H), 3.91-3.82 (m, 1H), 2.83 (d, J=4.63 Hz, 3H), 2.47 (s, 3H), 2.42-2.25 (m, 2H), 1.99 (s, 3H), 1.33 (br s, 2H), 1.17 (br s, 2H).

### Example 321: 2-Methyl-5-((1-methylazetidin-2-yl)methoxy)-N-(1-(3-(prop-1-en-2-yl)

naphthalen-1-yl)cyclopropyl)benzamide (Compound 508)

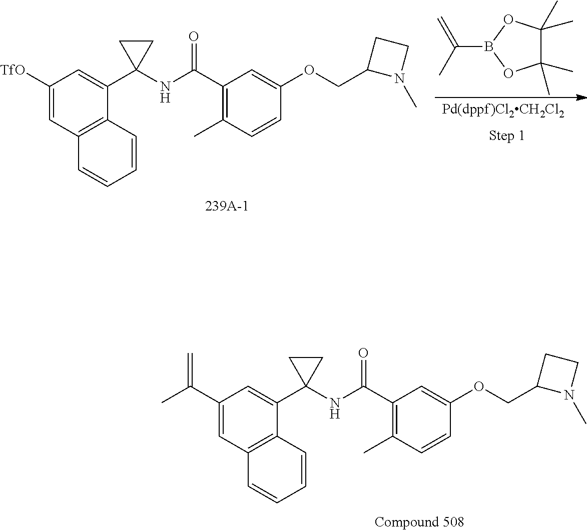

### Step 1: 2-Methyl-5-((1-methylazetidin-2-yl)methoxy)-N-(1-(3-(prop-1-en-2-yl) naphthalen-1-yl)cyclopropyl)benzamide (Compound 508)

To a solution of 4-(1-(2-methyl-5-((1-methylazetidin-2-yl)methoxy)benzamido)cyclopropyl) naphthalen-2-yl trifluoromethanesulfonate (200 mg, 365 μmol, 1.0 eq) and 4,4,5,5-tetramethyl-2-(prop-1-en-2-yl)-1,3,2-dioxaborolane (129 mg, 766 μmol, 2.1 eq) in a mixture of dioxane (12 mL) and H₂O (2.0 mL) were added Pd(dppf)Cl₂·CH₂Cl₂ (29.8 mg, 36.5 μmol, 0.1 eq) and Na₂CO₃ (δ8.9 mg, 839 μmol, 2.3 eq). The mixture was degassed and purged with N₂ three times, and then the mixture was stirred at 80° C. for 14 h under a N₂ atmosphere. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over dried over Na₂SO₄, filtered, and concentrated under vacuum to give a residue which was purified by preparative HPLC (Phenomenex Luna column (80×30 mm, 3 μm); flow rate: 25 mL/min; gradient: 5%-45% B over 8 min; mobile phase A: 0.04% aqueous HCl, mobile phase B: acetonitrile). 2-Methyl-5-((1-methylazetidin-2-yl)methoxy)-N-(1-(3-(prop-1-en-2-yl) naphthalen-1-yl)cyclopropyl)benzamide (18.4 mg, 40.9 μmol, 11% yield, HCl salt) was obtained as a yellow oil. M+H⁺=441.1 (LCMS); ¹H NMR (400 MHZ, DMSO-d₆) δ 10.24-10.11 (m, 1H), 9.11 (s, 1H), 8.62 (d, J=7.6 Hz, 1H), 8.03 (d, J=1.6 Hz, 1H), 7.99-7.93 (m, 1H), 7.89 (s, 1H), 7.58-7.48 (m, 2H), 7.09 (d, J=8.5 Hz, 1H), 6.90 (dd, J=2.5, 8.1 Hz, 1H), 6.69 (d, J=2.4 Hz, 1H), 5.62 (s, 1H), 5.25 (s, 1H), 4.66-4.55 (m, 1H), 4.31-4.18 (m, 2H), 4.05-3.79 (m, 2H), 2.86-2.65 (m, 3H), 2.39-2.22 (m, 5H), 1.99 (s, 3H), 1.37 (br s, 2H), 1.23 (br s, 2H).

### Example 322: N-(1-(3-Isopropylnaphthalen-1-yl)cyclopropyl)-2-methyl-5-((1-methyl

azetidin-2-yl)methoxy)benzamide (Compound 521)

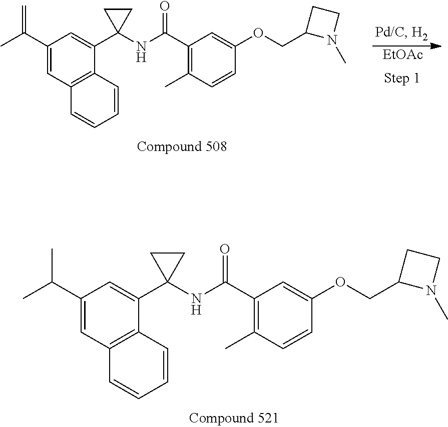

### Step 1: N-(1-(3-Isopropylnaphthalen-1-yl)cyclopropyl)-2-methyl-5-((1-methylazetidin-2-yl)methoxy)benzamide (Compound 521)

To a solution of 2-methyl-5-((1-methylazetidin-2-yl)methoxy)-N-(1-(3-(prop-1-en-2-yl)naphthalen-1-yl)cyclopropyl)benzamide (90.0 mg, 204 μmol, 1.0 eq) in EtOAc (7.0 mL) was added 10% palladium on carbon (90.0 mg). The suspension was degassed and purged with H₂ several times. The mixture was stirred at 20° C. for 2 h under a H₂ (15 psi) atmosphere.

LCMS indicated that the starting material was completely consumed, and the desired mass was detected. The reaction mixture was filtered through a pad of Celite and the filtrate was concentrated under vacuum to give a residue which was purified by preparative HPLC (Phenomenex Luna column (80×30 mm, 3 μm); flow rate: 25 mL/min; gradient: 10%-50% B over 8 min; mobile phase A: 0.04% aqueous HCl, mobile phase B: acetonitrile). N-(1-(3-Isopropylnaphthalen-1-yl)cyclopropyl)-2-methyl-5-((1-methylazetidin-2-yl)methoxy)benzamide (18.0 mg, 37.0 μmol, 18% yield, HCl salt) was obtained as a red solid. M+H⁺=443.2 (LCMS); ¹H NMR (400 MHZ, DMSO-d₆) δ 10.13-10.01 (m, 1H), 9.06 (s, 1H), 8.60 (br d, J=6.6 Hz, 1H), 7.87 (br d, J=8.6 Hz, 1H), 7.75 (s, 1H), 7.64 (s, 1H), 7.52-7.44 (m, 2H), 7.09 (br d, J=8.8 Hz, 1H), 6.90 (br dd, J=1.4, 7.9 Hz, 1H), 6.69 (br s, 1H), 4.59 (br dd, J=3.0, 7.8 Hz, 1H), 4.29-4.17 (m, 2H), 4.06-3.94 (m, 1H), 3.90-3.77 (m, 1H), 3.08-3.01 (m, 1H), 2.81 (br s, 3H), 2.37-2.32 (m, 2H), 2.00 (s, 3H), 1.35 (br s, 2H), 1.31 (br d, J=6.8 Hz, 6H), 1.19 (br s, 2H).

### Example 323: 5-(2-(Dimethylamino)ethoxy)-N-(1-(3-(ethylsulfinyl)naphthalen-1-yl)

cyclopropyl)-2-methylbenzamide (Compound 423)

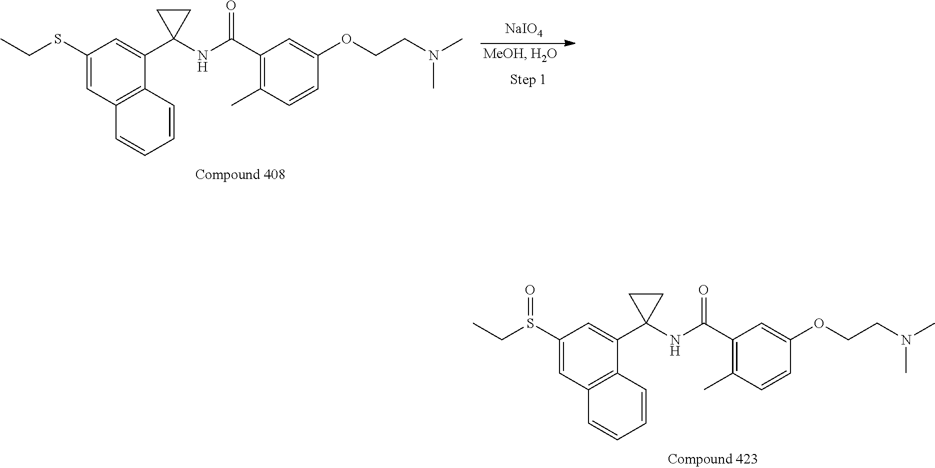 [SMILES: CCSc1cc(C2(NC(=O)c3cc(OCCN(C)C)ccc3C)CC2)c2ccccc2c1 ; "NaIO4" ; CCS(=O)c1cc(C2(NC(=O)c3cc(OCCN(C)C)ccc3C)CC2)c2ccccc2c1]

### Step 1: 5-(2-(Dimethylamino)ethoxy)-N-(1-(3-(ethylsulfinyl)naphthalen-1-yl)cyclopropyl)-2-methylbenzamide (Compound 423)

To a solution of 5-(2-(dimethylamino)ethoxy)-N-(1-(3-(ethylthio) naphthalen-1-yl)cyclo propyl)-2-methylbenzamide (60.0 mg, 134 μmol, 1.0 eq) in MeOH (300 μL) were added NaIO₄ (28.6 mg, 134 μmol, 1.0 eq) and H₂O (1.5 mL). The mixture was stirred at 20° C. for 2 h. LCMS indicated that the starting material was completely consumed, and the desired mass was detected. The mixture was treated with saturated aqueous NaHSO₃ (5.0 mL) and extracted with DCM (5.0 mL×3). The combined organic layers were dried over Na₂SO₄ filtered, and concentrated under vacuum to give a residue which was purified by preparative HPLC (Phenomenex Luna C18 column (75×30 mm, 3 μm); flow rate: 25 mL/min; gradient: 12%-42% B over 8 min; mobile phase A: 0.2% aqueous FA, mobile phase B: acetonitrile). 5-(2-(Dimethylamino)ethoxy)-N-(1-(3-(ethylsulfinyl)naphthalen-1-yl)cyclopropyl)-2-methyl benzamide (20.2 mg, 39.3 μmol, 29% yield, FA salt) was obtained as a white solid. M+H⁺=465.1 (LCMS); ¹H NMR (400 MHZ, CDCl₃) δ 8.57-8.51 (m, 1H), 8.45-8.40 (m, 1H), 8.24-8.20 (m, 1H), 8.03-7.95 (m, 2H), 7.71-7.57 (m, 2H), 7.03-6.97 (m, 1H), 6.83-6.70 (m, 3H), 4.16-4.04 (m, 2H), 3.14-3.00 (m, 1H), 2.97-2.81 (m, 3H), 2.48 (s, 6H), 2.08 (s, 3H), 1.64-1.52 (m, 2H), 1.49-1.34 (m, 2H), 1.30-1.18 (m, 3H).

### Example 324: 5-(2-(Dimethylamino)ethoxy)-N-(1-(3-(ethylsulfonyl)naphthalen-1-yl)

cyclopropyl)-2-methylbenzamide (Compound 471)

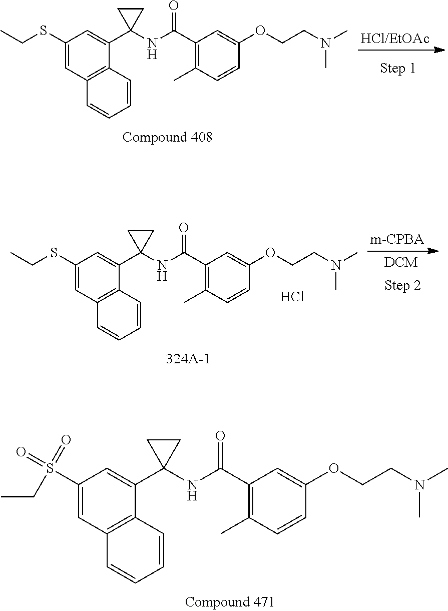

1 (Step 1: 5-(2-(Dimethylamino)ethoxy)-N-(1-(3-(ethylthio) naphthalen-1-yl)cyclopropyl)-2-methylbenzamide hydrochloride (324A-1)

To a solution of 5-(2-(dimethylamino)ethoxy)-N-(1-(3-(ethylthio) naphthalene-1-yl)cyclopropyl)-2-methylbenzamide (75.0 mg, 167 μmol, 1.0 eq) in EtOAc (1.0 mL) was added HCl/EtOAc (4 M, 6.0 mL). The mixture was stirred at 20° C. for 30 min. TLC indicated that the starting material was completely consumed. The mixture was concentrated under vacuum to give the crude product 5-(2-(dimethylamino)ethoxy)-N-(1-(3-(ethylthio) naphthalen-1-yl)cyclopropyl)-2-methylbenzamide hydrochloride (80.0 mg, HCl salt) as a white solid, which was used in the next step without any further purification.

### Step 2: 5-(2-(Dimethylamino)ethoxy)-N-(1-(3-(ethylsulfonyl)naphthalen-1-yl)cyclopropyl)-2-methylbenzamide (Compound 471)

To a solution of 5-(2-(dimethylamino)ethoxy)-N-(1-(3-(ethylthio) naphthalen-1-yl)cyclopropyl)-2-methylbenzamide hydrochloride (80.0 mg, 165 μmol, 1.0 eq, HCl salt) in DCM (2.0 mL) was added m-CPBA (83.7 mg, 412 μmol, 85% purity, 2.5 eq) at 0° C. The mixture was stirred at 20° C. for 2 h. LCMS indicated that the starting material was consumed, and the desired mass was detected. The mixture was treated with saturated aqueous NaHSO₃ (10 mL) and extracted with DCM (5.0 mL×5). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 20%-55% B over 8 min; mobile phase A: 0.2% aqueous FA, mobile phase B: acetonitrile). 5-(2-(Dimethylamino)ethoxy)-N-(1-(3-(ethylsulfonyl)naphthalen-1-yl)cyclo propyl)-2-methyl benzamide (4.30 mg, 8.55 μmol, 5% yield, FA salt) was obtained as a white solid. M+H⁺=481.1 (LCMS); ¹H NMR (400 MHZ, CDCl₃) δ 8.77-8.61 (m, 1H), 8.47-8.43 (m, 1H), 8.36-8.30 (m, 2H), 8.08-8.02 (m, 1H), 7.82-7.74 (m, 1H), 7.71-7.62 (m, 1H), 7.11-6.94 (m, 2H), 6.84-6.56 (m, 2H), 4.33-4.18 (m, 2H), 3.30-3.22 (m, 2H), 3.20-3.12 (m, 2H), 2.75-2.61 (m, 6H), 2.19-2.03 (m, 3H), 1.64-1.56 (m, 2H), 1.48-1.41 (m, 2H), 1.37-1.31 (m, 3H).

### Example 325: 2-Methyl-5-(2-(methylamino)ethoxy)-N-(1-(3-(thiophen-2-yl)naphthalen-1-yl)cyclopropyl)benzamide (Compound 486)

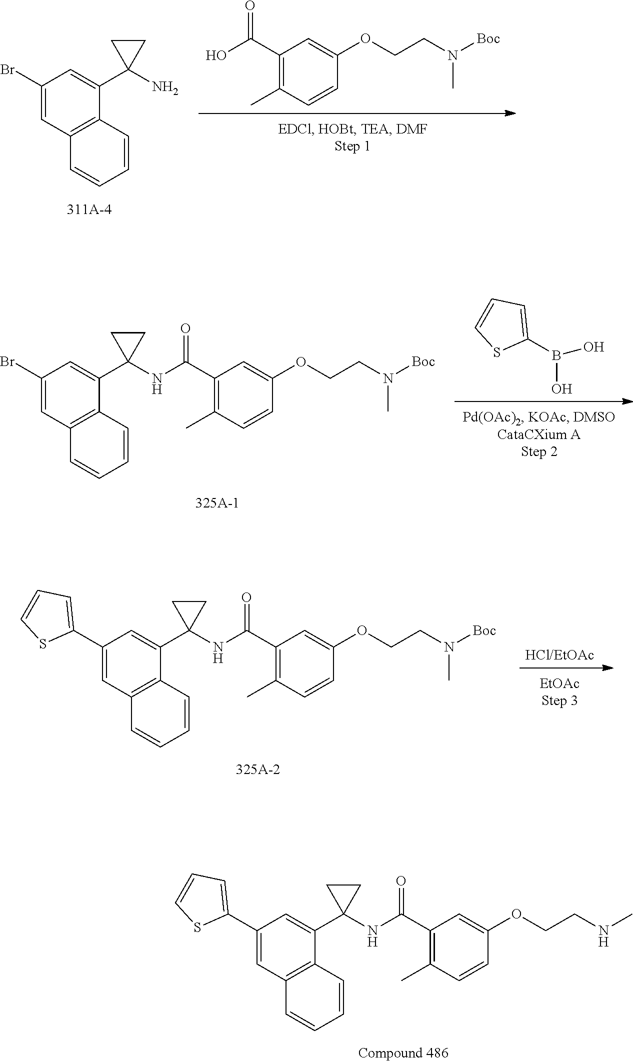

### Step 1: tert-Butyl(2-(3-((1-(3-bromonaphthalen-1-yl)cyclopropyl)carbamoyl)-4-methyl phenoxy)ethyl)(methyl)carbamate (325A-1)

To a solution of 5-(2-((tert-butoxycarbonyl)(methyl)amino)ethoxy)-2-methylbenzoic acid (236 mg, 763 μmol, 1.0 eq) in DMF (15 mL) were added 1-(3-bromonaphthalen-1-yl)cyclo propanamine (200 mg, 763 μmol, 1.0 eq), EDCI (219 mg, 1.14 mmol, 1.5 eq), HOBt (155 mg, 1.14 mmol, 1.5 eq) and TEA (154 mg, 153 mmol, 212 μL, 2.0 eq). The mixture was stirred at 20° C. for 16 h. LCMS indicated that the starting material was completely consumed, and the desired mass was detected. The reaction mixture was poured into H₂O (10 mL) and extracted with EtOAc (8.0 mL×3). The combined organic layers were dried over Na₂SO₄, filtered, and concentrated under vacuum to give a residue which was purified by flash silica gel chromatography using a gradient of EtOAc/petroleum ether from 1/100 to 4/5. tert-Butyl(2-(3-((1-(3-bromonaphthalen-1-yl)cyclopropyl)carbamoyl)-4-methylphenoxy)ethyl)(methyl) carbamate (340 mg, 614 μmol, 81% yield) was obtained as a white solid. M+H⁺=553.1 (LCMS).

### Step 2: tert-Butyl methyl(2-(4-methyl-3-((1-(3-(thiophen-2-yl)naphthalen-1-yl)cyclo propyl)carbamoyl)phenoxy)ethyl)carbamate (325A-2)

To a solution of tert-butyl(2-(3-((1-(3-bromonaphthalen-1-yl)cyclopropyl)carbamoyl)-4-methyl phenoxy)ethyl)(methyl)carbamate (200 mg, 361 μmol, 1.0 eq) in DMSO (10 mL) were added thiophen-2-ylboronic acid (55.5 mg, 434 μmol, 1.2 eq), Pd(OAc) 2 (8.11 mg, 36.1 μmol, 0.1 eq), KOAc (106 mg, 1.08 mmol, 3.0 eq) and CataCXium A (25.9 mg, 72.3 μmol, 0.2 eq). The mixture was degassed and purged with N₂ three times. The resulting mixture was stirred at 80° C. under a N₂ atmosphere for 16 h. LCMS indicated that the starting material was completely consumed, and the desired mass was detected. The reaction mixture was allowed to cool to room temperature, poured into H₂O (6.0 mL) and extracted with EtOAc (4.0 mL×3). The combined organic layers were dried over Na₂SO₄, filtered, and concentrated under vacuum to give a residue which was purified by preparative TLC (EtOAc/petroleum ether=1/3, R_{f}=0.4). tert-Butyl methyl(2-(4-methyl-3-((1-(3-(thiophen-2-yl)naphthalen-1-yl)cyclopropyl)carbamoyl)phenoxy)ethyl)carbamate (74.0 mg, 133 μmol, 37% yield) was obtained as a white solid. M+H⁺=557.3 (LCMS).

### Step 3: 2-Methyl-5-(2-(methylamino)ethoxy)-N-(1-(3-(thiophen-2-yl)naphthalen-1-yl)cyclopropyl)benzamide (Compound 486)

To a solution of tert-butyl methyl(2-(4-methyl-3-((1-(3-(thiophen-2-yl)naphthalen-1-yl)cyclo propyl)carbamoyl)phenoxy)ethyl)carbamate (74.0 mg, 133 μmol, 1.0 eq) in EtOAc (1.5 mL) was added HCl/EtOAc (4 M, 7.4 mL). The mixture was stirred at 20° C. for 1 h. LCMS indicated that the starting material was completely consumed, and the desired mass was detected. The mixture was concentrated under vacuum at 20° C. to give a residue which was purified by preparative HPLC (Phenomenex Luna column (80×30 mm, 3 μm); flow rate: 25 mL/min; gradient: 20%-50% B over 8 min; mobile phase A: 0.04% aqueous HCl, mobile phase B: acetonitrile). 2-Methyl-5-(2-(methylamino)ethoxy)-N-(1-(3-(thiophen-2-yl)naphthalen-1-yl)cyclopropyl)benzamide (36.4 mg, 71.5 μmol, 54% yield, HCl salt) was obtained as a white solid. M+H⁺=457.1 (LCMS); ¹H NMR (400 MHZ, DMSO-d₆) δ 9.19 (s, 1H), 8.76-8.65 (m, 2H), 8.62 (br d, J=8.9 Hz, 1H), 8.11 (d, J=7.6 Hz, 2H), 8.03-7.96 (m, 1H), 7.66 (d, J=3.1 Hz, 1H), 7.65-7.60 (m, 1H), 7.59-7.52 (m, 2H), 7.24-7.19 (m, 1H), 7.09 (d, J=8.4 Hz, 1H), 6.93-6.86 (m, 1H), 6.66 (d, J=2.6 Hz, 1H), 4.13 (t, J=4.8 Hz, 2H), 3.30-3.22 (m, 2H), 2.58 (br t, J=5.1 Hz, 3H), 1.99 (s, 3H), 1.38 (br s, 2H), 1.27 (br s, 2H).

### Example 326: (S)-5-(2-Aminopropoxy)-2-methyl-N-(1-(3-(thiophen-2-yl)naphthalen-1-yl)cyclopropyl)benzamide (Compound 539)

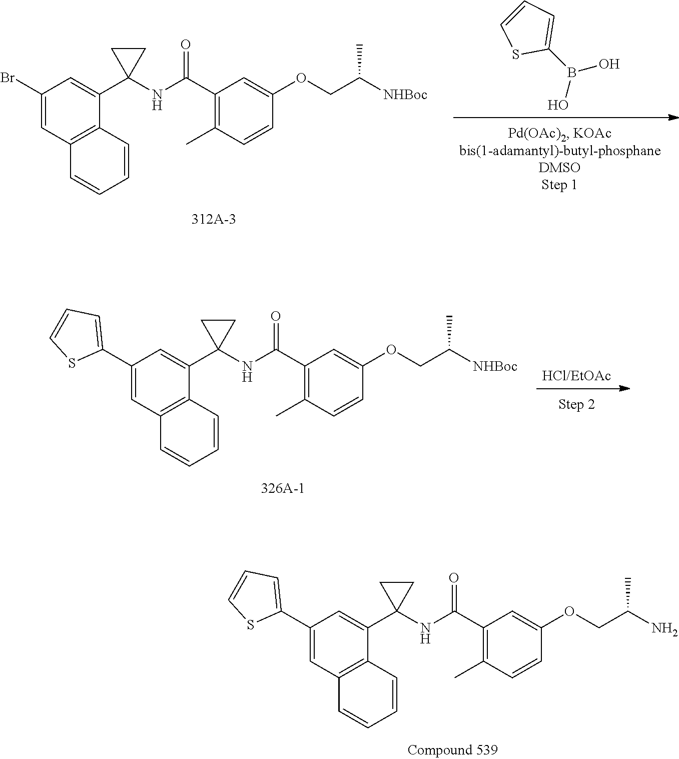 [SMILES: Cc1ccc(OC[C@H](C)NC(=O)OC(C)(C)C)cc1C(=O)NC1(c2cc(Br)cc3ccccc23)CC1 ; OB(O)c1cccs1 ; Cc1ccc(OC[C@H](C)NC(=O)OC(C)(C)C)cc1C(=O)NC1(c2cc(-c3cccs3)cc3ccccc23)CC1 ; "HCl/EtOAc" ; Cc1ccc(OC[C@H](C)N)cc1C(=O)NC1(c2cc(-c3cccs3)cc3ccccc23)CC1]

### Step 1: (S)-tert-Butyl(1-(4-methyl-3-((1-(3-(thiophen-2-yl)naphthalen-1-yl)cyclopropyl)carbamoyl)phenoxy)propan-2-yl)carbamate (326A-1)

To a solution of(S)-tert-butyl(1-(3-((1-(3-bromonaphthalen-1-yl)cyclopropyl)carbamoyl)-4-methylphenoxy)propan-2-yl)carbamate (350 mg, 632 μmol, 1.0 eq) and thiophen-2-ylboronic acid (121 mg, 948 μmol, 1.5 eq) in DMSO (3.0 mL) were added Pd(OAc)₂ (14.2 mg, 63.2 μmol, 0.1 eq), bis(1-adamantyl)-butyl-phosphane (45.3 mg, 126 μmol, 0.2 eq) and KOAc (186 mg, 1.90 mmol, 3.0 eq). The resulting mixture was stirred at 80° C. under a N₂ atmosphere for 16 h. LCMS indicated that the starting material was completely consumed. The reaction mixture was poured into H₂O (30 mL) and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 0/1 to 1/5. (S)-tert-Butyl(1-(4-methyl-3-((1-(3-(thiophen-2-yl)naphthalen-1-yl)cyclopropyl)carbamoyl)phenoxy)propan-2-yl)carbamate (290 mg, 500 μmol, 79% yield) was obtained as a white solid. M−56+H⁺=501.3 (LCMS).

### Step 2: (S)-5-(2-Aminopropoxy)-2-methyl-N-(1-(3-(thiophen-2-yl)naphthalen-1-yl)cyclopropyl)benzamide (Compound 539)

To a solution of(S)-tert-butyl(1-(4-methyl-3-((1-(3-(thiophen-2-yl)naphthalen-1-yl)cyclopropyl)carbamoyl)phenoxy)propan-2-yl)carbamate (290 mg, 521 μmol, 1.0 eq) in EtOAc (2.0 mL) was added HCl/EtOAc (4 M, 5.0 mL). The mixture was stirred at 20° C. for 1 h. LCMS indicated that the starting material was completely consumed, and the desired mass was detected. The mixture was concentrated under vacuum to give a residue which was purified by preparative HPLC (Phenomenex Luna C18 column (80×30 mm, 3 μm); flow rate: 25 mL/min; gradient: 20%-60% B over 8 min; mobile phase A: 0.04% aqueous HCl, mobile phase B:

acetonitrile). (S)-5-(2-Aminopropoxy)-2-methyl-N-(1-(3-(thiophen-2-yl)naphthalen-1-yl)cyclopropyl)benzamide (161 mg, 327 μmol, 63% yield, HCl salt) was obtained as a white solid. M+H⁺=457.1 (LCMS); ¹H NMR (400 MHZ, DMSO-d₆) δ 9.18 (s, 1H), 8.66-8.60 (m, 1H), 8.13-8.06 (m, 4H), 8.03-7.98 (m, 1H), 7.67-7.65 (m, 1H), 7.62 (dd, J=1.0, 5.1 Hz, 1H), 7.55 (s, 2H), 7.24-7.19 (m, 1H), 7.10-7.05 (m, 1H), 6.91-6.86 (m, 1H), 6.67 (d, J=2.8 Hz, 1H), 4.06-3.99 (m, 1H), 3.91-3.84 (m, 1H), 3.59-3.47 (m, 1H), 1.99 (s, 3H), 1.39 (br s, 2H), 1.27 (br s, 2H), 1.23 (d, J=6.8 Hz, 3H).

### Example 327: 2-Methyl-5-((1-methylazetidin-2-yl)methoxy)-N-(1-(3-(thiophen-2-yl)naphthalen-1-yl)cyclopropyl)benzamide (Compound 468)

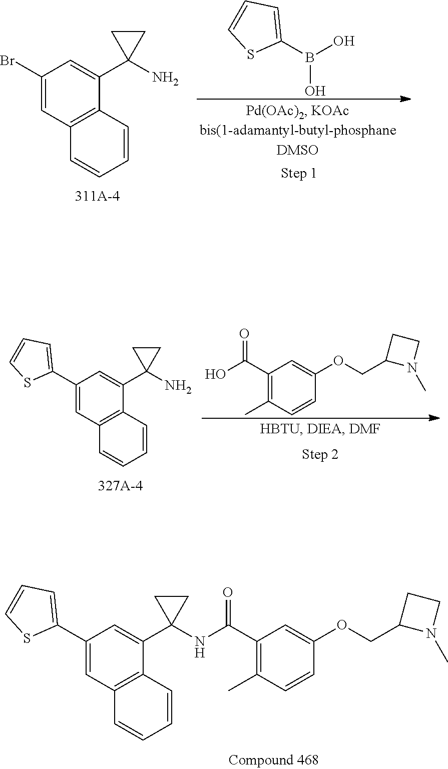 [SMILES: NC1(c2cc(Br)cc3ccccc23)CC1 ; OB(O)c1cccs1 ; NC1(c2cc(-c3cccs3)cc3ccccc23)CC1 ; Cc1ccc(OCC2CCN2C)cc1C(=O)O ; Cc1ccc(OCC2CCN2C)cc1C(=O)NC1(c2cc(-c3cccs3)cc3ccccc23)CC1]

### Step 1: 1-(3-(Thiophen-2-yl)naphthalen-1-yl)cyclopropanamine (327A-1)

To a solution of 1-(3-bromonaphthalen-1-yl)cyclopropanamine (160 mg, 610 μmol, 1.0 eq) and thiophen-2-ylboronic acid (93.7 mg, 732 μmol, 1.2 eq) in DMSO (1.5 mL) were added Pd(OAc)₂ (13.7 mg, 61.0 μmol, 0.1 eq), bis(1-adamantyl)-butyl-phosphane (43.7 mg, 122 μmol, 0.2 eq) and KOAc (179 mg, 1.83 mmol, 3.0 eq). The mixture was stirred at 80° C. under a N₂ atmosphere for 16 h. LCMS indicated that the starting material was completely consumed. The reaction mixture was allowed to cool to room temperature, poured into water (10 mL) and extracted with EtOAc (8.0 mL×3). The combined organic layers were dried over Na₂SO₄. filtered, and concentrated under vacuum to give a crude product 1-(3-(thiophen-2-yl)naphthalen-1-yl)cyclopropanamine (402 mg) as a black oil. M+H⁺=266.1 (LCMS).

### Step 2: 2-Methyl-5-((1-methylazetidin-2-yl)methoxy)-N-(1-(3-(thiophen-2-yl)naphthalen-1-yl)cyclopropyl)benzamide (Compound 468)

To a solution of 1-(3-(thiophen-2-yl)naphthalen-1-yl)cyclopropanamine (28.0 mg, 105 μmol, 1.0 eq) and 2-methyl-5-((1-methylazetidin-2-yl)methoxy)benzoic acid (27.3 mg, 116 μmol, 1.1 eq) in DMF (1.0 mL) were added HBTU (80.0 mg, 211 μmol, 2.0 eq) and DIEA (54.5 mg, 422 μmol, 73.5 μL, 4.0 eq). The mixture was stirred at 20° C. for 2 h. LCMS indicated that the starting material was completely consumed. The reaction mixture was filtered. The filtrate was purified by preparative HPLC (Phenomenex Luna C18 column (75×30 mm, 3 μm); flow rate: 25 mL/min; gradient: 20%-50% B over 8 min; mobile phase A: 0.1% aqueous TFA, mobile phase B: acetonitrile). 2-Methyl-5-((1-methylazetidin-2-yl)methoxy)-N-(1-(3-(thiophen-2-yl)naphthalen-1-yl)cyclopropyl)benzamide (6.40 mg, 10.6 μmol, 10% yield, TFA salt) was obtained as a yellow solid. M+H⁺=483.2 (LCMS); ¹H NMR (400 MHZ, CD₃OD) δ 9.26 (s, 1H), 8.55 (d, J=8.00 Hz, 1H), 8.25 (d, J=1.63 Hz, 1H), 8.05 (s, 1H), 7.97-7.88 (m, 1H), 7.62-7.58 (m, 1H), 7.55-7.51 (m, 1H), 7.43 (d, J=4.63 Hz, 1H), 7.19-7.06 (m, 2H), 6.93 (dd, J=8.44, 2.56 Hz, 1H), 6.74 (d, J=2.50 Hz, 1H), 4.71-4.59 (m, 1H), 4.32-4.22 (m, 1H), 4.21-4.08 (m, 2H), 3.99-3.87 (m, 1H), 2.92 (s, 3H), 2.59-2.47 (m, 2H), 2.05 (s, 3H), 1.53-1.45 (m, 2H), 1.41-1.33 (m, 2H).

### Example 328: 5-(2-Aminopropoxy)-2-methyl-N-(1-(3-phenylnaphthalen-1-yl)cyclopropyl)benzamide (Compound 470)

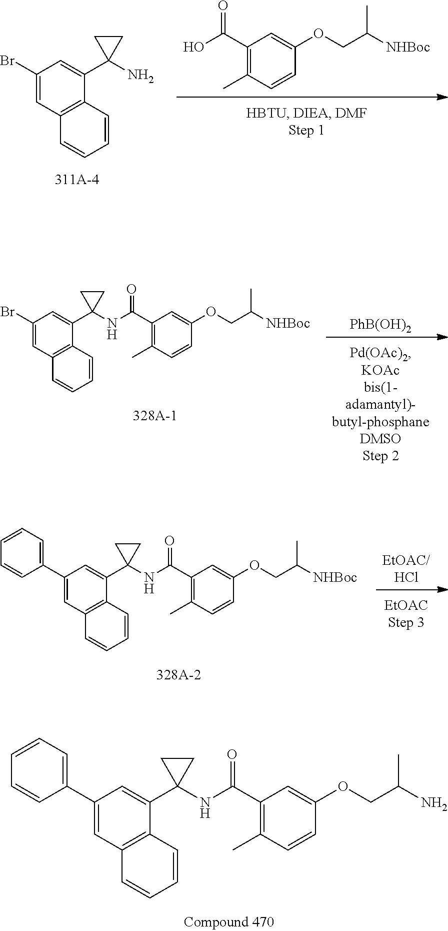

### Step 1: tert-Butyl(1-(3-((1-(3-bromonaphthalen-1-yl)cyclopropyl)carbamoyl)-4-methylphenoxy)propan-2-yl)carbamate (328A-1)

To a solution of 1-(3-bromonaphthalen-1-yl)cyclopropanamine (350 mg, 1.34 mmol, 1.0 eq) in DMF (2.0 mL) was added 5-(2-((tert-butoxycarbonyl)amino)propoxy)-2-methylbenzoic acid (454 mg, 1.47 mmol, 1.1 eq), followed by HBTU (1.01 g, 2.67 mmol, 2.0 eq) and DIEA (518 mg, 4.01 mmol, 698 μL, 3.0 eq). The mixture was stirred at 20° C. for 16 h. LCMS indicated that the starting material was completely consumed, and the desired mass was detected. The reaction mixture was poured into H₂O (10 mL) and extracted with EtOAc (8.0 mL×4). The combined organic layers were dried over Na₂SO₄, filtered, and concentrated under vacuum to give a crude product tert-butyl(1-(3-((1-(3-bromonaphthalen-1-yl)cyclopropyl)carbamoyl)-4-methylphenoxy)propan-2-yl)carbamate (1.64 g) as a yellow oil. M−56+H⁺=479.1 (LCMS).

### Step 2: tert-Butyl(1-(4-methyl-3-((1-(3-phenylnaphthalen-1-yl)cyclopropyl)carbamoyl)phenoxy)propan-2-yl)carbamate (328A-2)

To a solution of tert-butyl(1-(3-((1-(3-bromonaphthalen-1-yl)cyclopropyl)carbamoyl)-4-methylphenoxy)propan-2-yl)carbamate (55.0 mg, 99.4 μmol, 1.0 eq) and phenylboronic acid (18.2 mg, 149 μmol, 1.5 eq) in DMSO (1.0 mL) were added Pd(OAc) 2 (2.23 mg, 9.94 μmol, 0.1 eq), bis(1-adamantyl)-butyl-phosphane (7.13 mg, 19.9 μmol, 0.2 eq) and KOAc (29.3 mg, 298 μmol, 3.0 eq). The mixture was stirred at 80° C. for 16 h under a N₂ atmosphere. LCMS indicated that 3% starting material remained and 44% desired compound was detected. The reaction mixture was allowed to cool to room temperature, poured into H₂O (10 mL) and extracted with EtOAc (8.0 mL×3). The combined organic layers were dried over Na₂SO₄, filtered, and concentrated under vacuum to give a crude product tert-butyl(1-(4-methyl-3-((1-(3-phenylnaphthalen-1-yl)cyclopropyl)carbamoyl)phenoxy)propan-2-yl)carbamate (110 mg) as a black oil. M−56+H⁺=495.2 (LCMS).

### Step 3: 5-(2-Aminoethoxy)-N-(1-(3-methoxynaphthalen-1-yl)cyclopropyl)-2-methyl benzamide (Compound 470)

To a solution of tert-butyl(1-(4-methyl-3-((1-(3-phenylnaphthalen-1-yl)cyclopropyl) carbamoyl)phenoxy)propan-2-yl)carbamate (110 mg, 200 μmol, 1.0 eq) in EtOAc (200 μL) was added HCl/EtOAc (4 M, 500 μL). The mixture was stirred at 20° C. for 30 min. LCMS indicated that the starting material was completely consumed, and the desired mass was detected. The mixture was concentrated under vacuum at 30° C. to give a residue which was purified by preparative HPLC (Phenomenex C18 column (80×30 mm, 3 μm); flow rate: 25 mL/min; gradient: 25%-55% B over 8 min; mobile phase A: 0.1% aqueous TFA, mobile phase B: acetonitrile) to give 5-(2-aminoethoxy)-N-(1-(3-methoxynaphthalen-1-yl)cyclopropyl)-2-methyl benzamide (20.0 mg, 35.0 μmol, 16% yield, TFA salt) as a white solid. M+H⁺=451.2 (LCMS); ¹H NMR (400 MHZ, DMSO-d₆) δ 9.17 (s, 1H), 8.70-8.61 (m, 1H), 8.14 (s, 2H), 8.06-8.00 (m, 1H), 7.92-7.88 (m, 2H), 7.85-7.80 (m, 2H), 7.60-7.50 (m, 4H), 7.45-7.38 (m, 1H), 7.08 (d, J=8.6 Hz, 1H), 6.91-6.85 (m, 1H), 6.66 (d, J=2.8 Hz, 1H), 4.01 (dd, J=3.8, 10.3 Hz, 1H), 3.83 (dd, J=7.3, 10.4 Hz, 1H), 3.53 (br d, J=4.1 Hz, 2H), 1.99 (s, 3H), 1.41-0.35 (m, 2H), 1.32-1.27 (m, 2H), 1.20 (d, J=6.8 Hz, 3H).

### Example 329: 2-Methyl-5-((1-methylazetidin-2-yl)methoxy)-N-(1-(3-phenylnaphthalen-1-yl)cyclopropyl)benzamide (Compound 475)

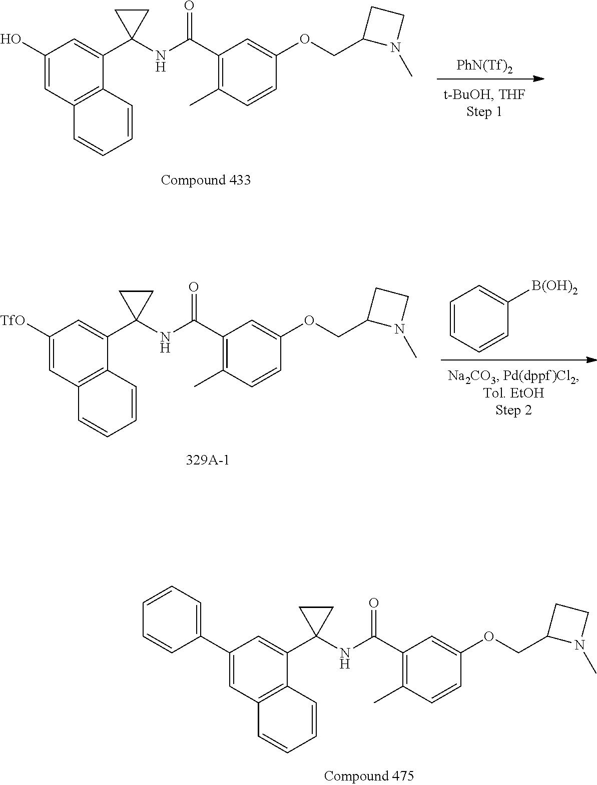

### Step 1:4-(1-(2-Methyl-5-((1-methylazetidin-2-yl)methoxy)benzamido)cyclopropyl)naphthalen-2-yl trifluoromethanesulfonate (329A-1)

To a solution of N-(1-(3-hydroxynaphthalen-1-yl)cyclopropyl)-2-methyl-5-((1-methyl azetidin-2-yl)methoxy)benzamide (200 mg, 480 μmol, 1.0 eq) in THF (10 mL) was added 1-BuOK (80.1 mg, 720 μmol, 1.5 eq) at 0° C. The mixture was stirred at 0° C. for 30 min. PhN(Tf)₂ (257 mg, 720 μmol, 1.5 eq) was added in portions. The resulting mixture was stirred at 20° C. for 16 h. LCMS indicated that the starting material was completely consumed, and the desired mass was detected. The mixture was poured into H₂O (10 mL) and extracted with DCM (10 mL×4). The combined organic layers were dried over Na₂SO₄ filtered, and concentrated under vacuum to give a residue which was purified by preparative TLC (MeOH/DCM=1/4, R_{f}=0.4). 4-(1-(2-Methyl-5-((1-methylazetidin-2-yl)methoxy)benzamido)cyclopropyl) naphthalen-2-yl trifluoromethanesulfonate (120 mg, 219 μmol, 46% yield) was obtained as a yellow solid. M+H⁺=549.2 (LCMS).

### Step 2: 2-Methyl-5-((1-methylazetidin-2-yl)methoxy)-N-(1-(3-phenylnaphthalen-1-yl)cyclopropyl)benzamide (Compound 475)

To a solution of 4-(1-(2-methyl-5-((1-methylazetidin-2-yl)methoxy)benzamido)cyclopropyl) naphthalen-2-yl trifluoromethanesulfonate (100 mg, 182 μmol, 1.0 eq) and phenylboronic acid (27.8 mg, 228 μmol, 1.3 eq) in a mixture of toluene (5.0 mL) and EtOH (2.5 mL) were added Na₂CO₃ (2 M aqueous, 228 μL, 2.5 eq) and Pd(dppf)Cl₂ (6.67 mg, 9.11 μmol, 0.05 eq) under a N₂ atmosphere. The mixture was stirred at 80° C. for 16 h. LCMS indicated that the starting material was completely consumed, and the desired mass was detected. The reaction mixture was allowed to cool to room temperature, poured into water (10 mL) and extracted with DCM (5.0 mL×4). The combined organic layers were dried over Na₂SO₄, filtered, and concentrated under vacuum to give a residue which was purified by preparative HPLC (Phenomenex Luna C18 column (200×40 mm, 10 μm); flow rate: 75 mL/min; gradient: 20%-50% B over 8 min; mobile phase A: 0.2% aqueous FA, mobile phase B: acetonitrile). 2-Methyl-5-((1-methylazetidin-2-yl)methoxy)-N-(1-(3-phenoxynaphthalen-1-yl)cyclopropyl)benzamide (18.1 mg, 34.6 μmol, 19% yield, FA salt) was obtained as a white solid. M+H⁺=477.1 (LCMS); ¹H NMR (400 MHZ, DMSO-d₆) δ 9.15-9.09 (m, 1H), 8.71-8.64 (m, 1H), 8.20-8.16 (m, 1H), 8.15-8.11 (m, 2H), 8.05-7.99 (m, 1H), 7.87-7.80 (m, 2H), 7.62-7.51 (m, 4H), 7.45-7.37 (m, 1H), 7.06-7.00 (m, 1H), 6.86-6.79 (m, 1H), 6.63-6.59 (m, 1H), 3.90-3.83 (m, 2H), 3.28-3.24 (m, 2H), 2.80-2.72 (m, 1H), 2.25-2.21 (m, 3H), 2.03-1.82 (m, 5H), 1.42-1.36 (m, 2H), 1.32-1.26 (m, 2H).

### Example 330: N-(1-(7-Hydroxynaphthalen-1-yl)cyclopropyl)-2-methyl-5-((1-methylazetidin-2-yl)methoxy)benzamide (Compound 477)

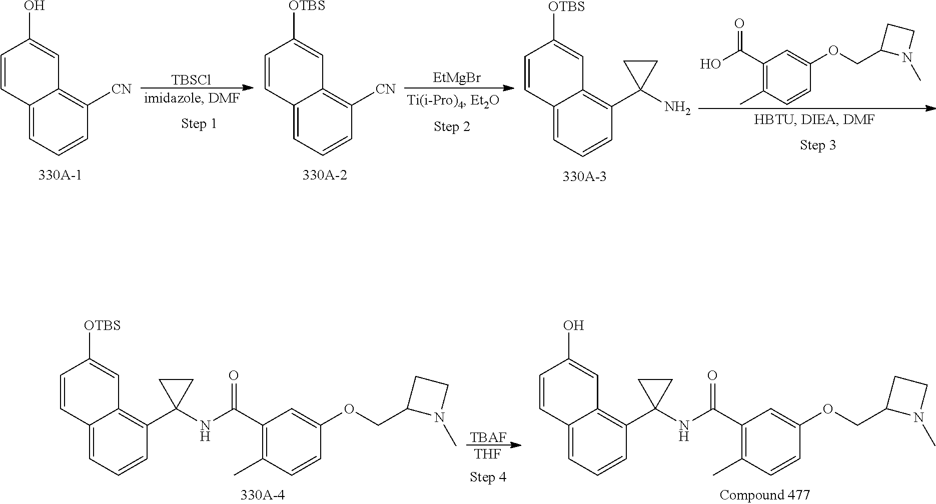

### Step 1: 7-((tert-Butyldimethylsilyl)oxy)-1-naphthonitrile (330A-2)

To a solution of 7-hydroxy-1-naphthonitrile (1.40 g, 8.28 mmol, 1.0 eq) in DMF (15 mL) were added TBSCl (1.50 g, 9.93 mmol, 1.22 mL, 1.2 eq) and imidazole (1.13 g, 16.6 mmol, 2.0 eq). The resulting mixture was stirred at 20° C. for 16 h. LCMS indicated that the starting material was completely consumed. The reaction mixture was poured into H₂O (30 mL) and extracted with EtOAc (20 mL×4). The combined organic layers were washed with brine (20 mL×2), dried over Na₂SO₄, filtered, and concentrated under vacuum to give a residue which was purified by flash silica gel chromatography using a gradient of EtOAc/petroleum ether from 0/1 to 1/5. 7-((ter-Butyldimethylsilyl)oxy)-1-naphthonitrile (1.60 g, 5.64 mmol, 68% yield) was obtained as a yellow gum. M+H⁺=284.1 (LCMS).

### Step 2: 1-(7-((tert-Butyldimethylsilyl)oxy) naphthalen-1-yl)cyclopropanamine (330A-3)

A solution of 7-((tert-butyldimethylsilyl)oxy)-1-naphthonitrile (800 mg, 2.82 mmol, 1.0 eq) in anhydrous Et₂O (70 mL) was degassed and purged with N₂ three times. To this mixture was added Ti(i-PrO)₄ (1.20 g, 4.23 mmol, 1.25 mL, 1.5 eq) slowly, and then EtMgBr (3 M in Et₂O, 2.07 mL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. BF₃·Et₂O (801 mg, 5.64 mmol, 697 μL, 2.0 eq) was added slowly with no obvious temperature change. The mixture was stirred at 20° C. for another 1 h. TLC indicated that the starting material was completely consumed, and the desired mass was detected. The reaction mixture was added into a mixture of HCl (1 M aqueous) (10 mL) and MTBE (10 mL) and extracted with MTBE (10 mL×2). The aqueous layer was basified to pH 8 by using NaOH (2 M aqueous), and a precipitate was formed. The mixture was filtered through a pad of Celite and the slurry was washed with DCM several times. The combined filtrate was extracted with DCM (10 mL×2). The combined organic layers were dried over Na₂SO₄, filtered, and concentrated under vacuum to give a residue which was purified by flash silica gel chromatography using a gradient of EtOAc/petroleum ether from 0/1 to 1/0. 1-(7-((tert-Butyldimethylsilyl)oxy) naphthalen-1-yl)cyclopropanamine (390 mg, 1.24 mmol, 44% yield) was obtained as a yellow gum.

### Step 3: N-(1-(7-((tert-Butyldimethylsilyl)oxy) naphthalen-1-yl)cyclopropyl)-2-methyl-5-((1-methylazetidin-2-yl)methoxy)benzamide (330A-4)

To a solution of 1-(7-((tert-butyldimethylsilyl)oxy) naphthalen-1-yl)cyclopropanamine (150 mg, 478 μmol, 1.0 eq) and 2-methyl-5-((1-methylazetidin-2-yl)methoxy)benzoic acid (146 mg, 622 μmol, 1.3 eq) in DMF (6.0 mL) were added HBTU (181 mg, 478 μmol, 1.0 eq) and DIEA (186 mg, 1.44 mmol, 250 μL, 3.0 eq). The mixture was stirred at 20° C. for 16 h. LCMS indicated that the starting material was completely consumed. The reaction mixture was poured into H₂O (10 mL) and extracted with EtOAc (10 mL×3). The combined organic layers were dried over Na₂SO₄, filtered, and concentrated under vacuum to give a residue which was purified by preparative TLC (DCM/MeOH=10/1, R_{f}=0.5). N-(1-(7-((tert-Butyldimethyl silyl)oxy) naphthalen-1-yl)cyclopropyl)-2-methyl-5-((1-methylazetidin-2-yl)methoxy)benzamide (150 mg, 283 μmol, 59% yield) was obtained as a yellow gum. M+H⁺=531.6 (LCMS).

### Step 4: N-(1-(7-Hydroxynaphthalen-1-yl)cyclopropyl)-2-methyl-5-((1-methylazetidin-2-yl)methoxy)benzamide (Compound 477)

To a solution of N-(1-(7-((tert-butyldimethylsilyl)oxy) naphthalen-1-yl)cyclopropyl)-2-methyl-5-((1-methylazetidin-2-yl)methoxy)benzamide (150 mg, 283 μmol, 1.0 eq) in THF (7.0 mL) was added TBAF (1 M in THF, 848 μL, 3.0 eq) at 0° C. The mixture was stirred at 20° C. for 3 h. LCMS indicated that the starting material was completely consumed. The reaction mixture was poured into H₂O (10 mL) and extracted with EtOAc (10 mL×4). The combined organic layers were washed with brine (10 mL×2), dried over Na₂SO₄, filtered, and concentrated under vacuum to give a residue which was purified by preparative TLC (DCM/MeOH=10/1, R_{f}=0.2). N-(1-(7-Hydroxynaphthalen-1-yl)cyclopropyl)-2-methyl-5-((1-methylazetidin-2-yl) methoxy)benzamide (6.30 mg, 14.7 μmol, 5% yield) was obtained as a white solid. M+H⁺=417.1 (LCMS); ¹H NMR (400 MHZ, DMSO-d₆) δ 9.60 (s, 1H), 9.02 (s, 1H), 7.84 (d, J=2.3 Hz, 1H), 7.77-7.64 (m, 3H), 7.20 (dd, J=7.2, 8.1 Hz, 1H), 7.12 (dd, J=2.3, 8.8 Hz, 1H), 7.03 (d, J=8.4 Hz, 1H), 6.83 (dd, J=2.8, 8.4 Hz, 1H), 6.63 (d, J=2.8 Hz, 1H), 3.89 (br d, J=5.3 Hz, 2H), 3.31-3.12 (m, 2H), 2.87-2.68 (m, 1H), 2.25 (s, 3H), 2.07-1.81 (m, 5H), 1.32 (s, 2H), 1.15-1.07 (m, 2H).

### Example 331:8-(1-(2-Methyl-5-((1-methylazetidin-2-yl)methoxy)benzamido)cyclopropyl)-2-naphthoic acid (Compound 465)

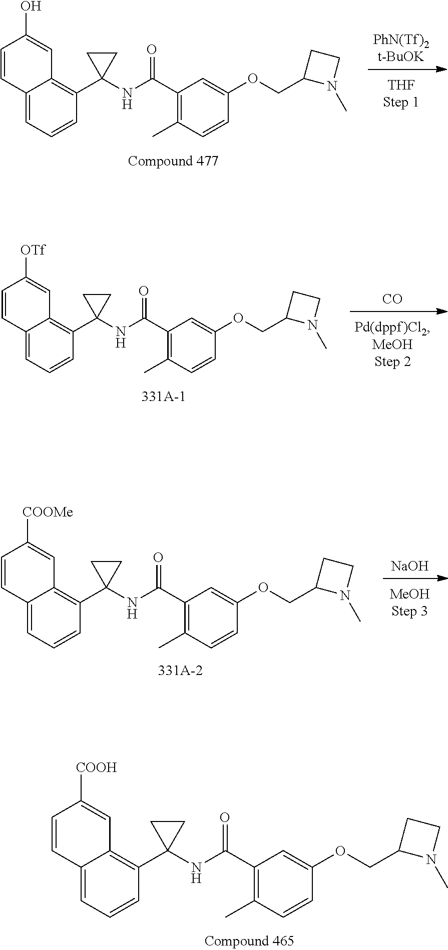

### Step 1:8-(1-(2-Methyl-5-((1-methylazetidin-2-yl)methoxy)benzamido)cyclopropyl)naphthalen-2-yl trifluoromethanesulfonate (331A-1)

To a solution of N-(1-(7-hydroxynaphthalen-1-yl)cyclopropyl)-2-methyl-5-((1-methylazetidin-2-yl)methoxy)benzamide (300 mg, 720 μmol, 1.0 eq) in THF (10 mL) was added PhN (Tf) 2 (386 mg, 1.08 mmol, 1.5 eq) at 0° C., followed by 1-BuOK (121 mg, 1.08 mmol, 1.5 eq). The resulting mixture was stirred at 20° C. for 16 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/0. 8-(1-(2-Methyl-5-((1-methylazetidin-2-yl)methoxy)benzamido)cyclopropyl) naphthalen-2-yl trifluoromethanesulfonate (300 mg, 547 μmol, 76% yield) was obtained as a yellow gum. M+H⁺=549.3 (LCMS).

### Step 2: Methyl 8-(1-(2-methyl-5-((1-methylazetidin-2-yl)methoxy)benzamido)cyclo propyl)-2-naphthoate (331A-2)

To a solution of 8-(1-(2-methyl-5-((1-methylazetidin-2-yl)methoxy)benzamido)cyclopropyl) naphthalen-2-yl trifluoromethanesulfonate (100 mg, 182 μmol, 1.0 eq) in MeOH (10 mL) were added Pd(dppf)Cl₂ (13.3 mg, 18.2 μmol, 0.1 eq) and TEA (148 mg, 1.46 mmol, 203 μL, 8.0 eq). The suspension was degassed and purged with CO three times. The mixture was stirred at 80° C. for 16 h under a CO (50 psi) atmosphere. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by preparative TLC (DCM/MeOH=10/1, R_{f}=0.35). Methyl 8-(1-(2-methyl-5-((1-methylazetidin-2-yl)methoxy)benzamido)cyclopropyl)-2-naphthoate (70.0 mg, 153 μmol, 84% yield) was obtained as a yellow gum. M+H⁺=459.2 (LCMS); ¹H NMR (400 MHZ, CDCl₃) δ 9.09 (s, 1H), 8.06-7.93 (m, 2H), 7.86 (d, J=8.5 Hz, 1H), 7.76 (d, J=8.3 Hz, 1H), 7.51 (t, J=7.6 Hz, 1H), 6.92 (d, J=8.0 Hz, 1H), 6.79-6.61 (m, 3H), 3.98-3.80 (m, 5H), 3.65-3.38 (m, 2H), 2.47-2.34 (m, 2H), 2.19-2.05 (m, 1H), 2.02 (s, 3H), 1.98 (s, 3H), 1.51 (br s, 2H), 1.33 (br t, J=5.3 Hz, 2H).

### Step 3:8-(1-(2-Methyl-5-((1-methylazetidin-2-yl)methoxy)benzamido)cyclopropyl)-2-naphthoic acid (Compound 465)

To a solution of methyl 8-(1-(2-methyl-5-((1-methylazetidin-2-yl)methoxy)benzamido)cyclo propyl)-2-naphthoate (60.0 mg, 131 μmol, 1.0 eq) in a mixture of MeOH (1.0 mL) and THE (3.0 mL) was added NaOH (2 M aqueous, 262 μL, 4.0 eq). The reaction mixture was stirred at 70° C. for 16 h. LCMS indicated that the starting material was completely consumed. The reaction mixture was allowed to cool to room temperature, poured into H₂O (10 mL), and washed with MTBE (10 mL×3). The aqueous was adjusted to pH 6 with HCl (1 M aqueous) and the mixture was concentrated under vacuum to give a residue which was purified by preparative HPLC (Phenomenex luna C18 column (80× 40 mm, 3 μm); flow rate: 25 mL/min; gradient: 1%-40% B over 7 min; mobile phase A: 0.04% aqueous HCl, mobile phase B:

acetonitrile). 8-(1-(2-Methyl-5-((1-methylazetidin-2-yl)methoxy)benzamido)cyclo propyl)-2-naphthoic acid (11.0 mg, 22.9 μmol, 17% yield, HCl salt) was obtained as a white solid. M+H⁺=445.1 (LCMS); ¹H NMR (400 MHZ, DMSO-d₆) δ 13.18-12.83 (m, 1H), 10.56-10.21 (m, 1H), 9.29 (s, 2H), 8.14-7.83 (m, 4H), 7.61 (dd, J=7.3, 8.0 Hz, 1H), 7.08 (d, J=8.5 Hz, 1H), 6.90 (br d, J=6.5 Hz, 1H), 6.70 (br s, 1H), 4.72-4.50 (m, 1H), 4.40-4.15 (m, 2H), 4.08-3.76 (m, 2H), 2.81 (br s, 3H), 2.44-2.19 (m, 2H), 1.96 (s, 3H), 1.42 (br s, 2H), 1.21 (br s, 2H).

### Example 332: N-(1-(7-Aminonaphthalen-1-yl)cyclopropyl)-2-methyl-5-((1-methylazetidin-2-yl)methoxy)benzamide (Compound 483)

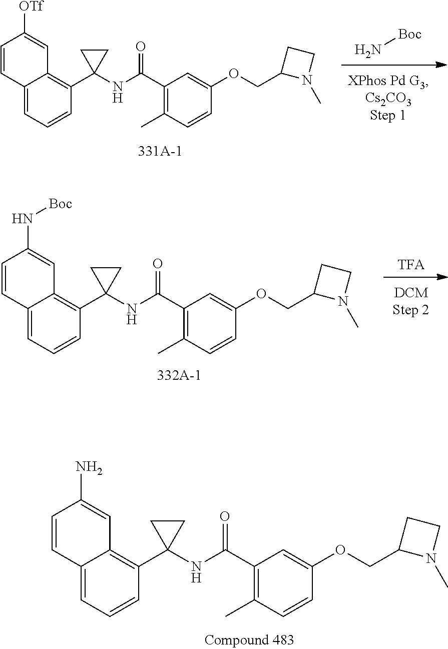 [SMILES: Cc1ccc(OCC2CCN2C)cc1C(=O)NC1(c2cccc3ccc(OS(=O)(=O)C(F)(F)F)cc23)CC1 ; CC(C)(C)OC(N)=O ; Cc1ccc(OCC2CCN2C)cc1C(=O)NC1(c2cccc3ccc(NC(=O)OC(C)(C)C)cc23)CC1 ; Cc1ccc(OCC2CCN2C)cc1C(=O)NC1(c2cccc3ccc(N)cc23)CC1 ; "TFA"]

### Step 1: tert-Butyl(8-(1-(2-methyl-5-((1-methylazetidin-2-yl)methoxy)benzamido)cyclopropyl)naphthalen-2-yl)carbamate (332A-1)

To a solution of 8-(1-(2-methyl-5-((1-methylazetidin-2-yl)methoxy)benzamido)cyclopropyl) naphthalen-2-yltrifluoromethanesulfonate (100 mg, 183 μmol, 182 μL, 1.0 eq) in 2-methylbutan-2-ol (4.0 mL) were added tert-butyl carbamate (25.6 mg, 219 μmol, 1.2 eq), Cs₂CO₃ (119 mg, 365 μmol, 2.0 eq) and XPhos Pd G3 (15.4 mg, 18.2 μmol, 0.1 eq) under a N₂ atmosphere. The mixture was stirred at 90° C. for 16 h. LCMS indicated that the starting material was completely consumed, and the desired compound was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by preparative TLC (DCM/MeOH=10/1, R_{f}=0.3). tert-Butyl(8-(1-(2-methyl-5-((1-methylazetidin-2-yl)methoxy)benzamido)cyclopropyl)naphthalen-2-yl)carbamate (60.0 mg, 116 μmol, 64% yield) was obtained as a yellow gum. M+H⁺=516.3 (LCMS).

### Step 2: N-(1-(7-Aminonaphthalen-1-yl)cyclopropyl)-2-methyl-5-((1-methylazetidin-2-yl)methoxy)benzamide (Compound 483)

To a solution of tert-butyl(8-(1-(2-methyl-5-((1-methylazetidin-2-yl)methoxy)benzamido)cyclopropyl)naphthalen-2-yl)carbamate (100 mg, 194 μmol, 1.0 eq) in DCM (8.0 mL) was added TFA (2.0 mL). The mixture was stirred at 20° C. for 1 h. LCMS indicated that the starting material was completely consumed, and the desired mass was detected. The reaction mixture was concentrated under vacuum to give a residue which was treated with NH₄OH (25% aqueous) to adjust pH to 7. The residue was purified by preparative HPLC (Waters Xbridge BEH C18 column (100×30 mm, 10 μm); flow rate: 25 mL/min; gradient: 15%-45% B over 8 min; mobile phase A: 10 mM aqueous NH₄HCO₃, mobile phase B: acetonitrile). N-(1-(7-Aminonaphthalen-1-yl)cyclopropyl)-2-methyl-5-((1-methylazetidin-2-yl)methoxy)benzamide (2.60 mg, 6.00 μmol, 3% yield) was obtained as a yellow solid. M+H⁺=416.2 (LCMS); 1H NMR (400 MHZ, DMSO-d₆) δ 8.80 (s, 1H), 7.66-7.48 (m, 4H), 7.10-7.00 (m, 2H), 6.98-6.73 (m, 2H), 6.61 (d, J=2.8 Hz, 1H), 5.09 (s, 2H), 3.86 (d, J=5.5 Hz, 2H), 3.25-3.17 (m, 2H), 2.73-2.65 (m, 1H), 2.21 (s, 3H), 2.08-1.76 (m, 5H), 1.29 (br s, 2H), 1.17-1.04 (m, 2H).

### Example 333: 5-(2-(Dimethylamino)ethoxy)-N-(1-(7-methoxyquinolin-5-yl)cyclopropyl)-2-methylbenzamide (Compound 434)

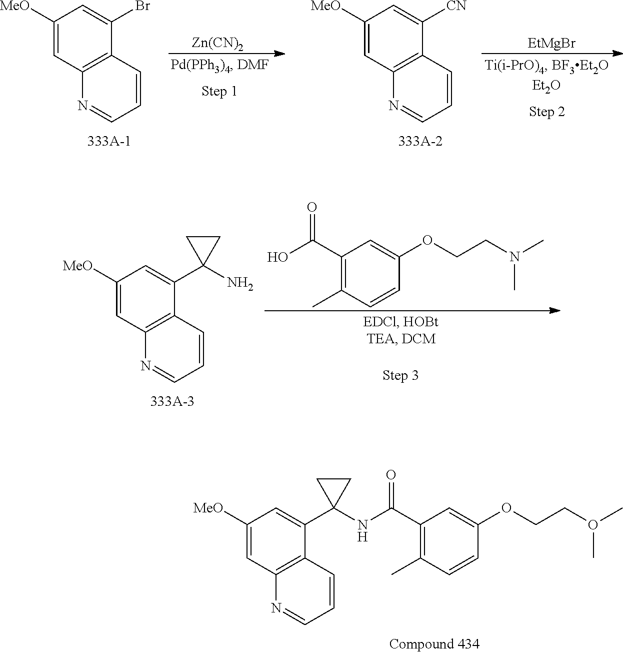

### Step 1: 7-Methoxyquinoline-5-carbonitrile (333A-2)

To a solution of 5-bromo-7-methoxyquinoline (10.0 g, 42.0 mmol, 1.0 eq) in DMF (250 mL) were added Zn(CN)₂ (9.86 g, 84.0 mmol, 2.0 eq) and Pd(PPh₃)₄ (4.85 g, 4.20 mmol, 0.1 eq). The mixture was degassed and purged with N₂ three times and stirred at 100° C. for 16 h under a N₂ atmosphere. LCMS indicated that the starting material was completely consumed, and the desired mass was detected. The reaction mixture was allowed to cool to room temperature and filtered. The filtrate was poured into H₂O (1.0 L) and extracted with EtOAc (500 mL×3). The combined organic layers were dried over Na₂SO₄, filtered, and concentrated under vacuum to the volume to 50 mL, and a precipitate was formed. The mixture was filtered, and the filter cake was washed with EtOAc (200 mL) to give a white solid. The solid was dissolved with DCM (300 mL) and stirred at room temperature for 30 min. The mixture was filtered, and the filtrate was concentrated under vacuum to give 7-methoxyquinoline-5-carbonitrile (6.25 g, 34.0 mmol, 81% yield) as a white solid.

### Step 2: 1-(7-Methoxyquinolin-5-yl)cyclopropanamine (333A-3)

A mixture of 7-methoxyquinoline-5-carbonitrile (2.00 g, 10.9 mmol, 1.0 eq) in anhydrous Et₂O (160 mL) was degassed and purged with N₂ three times. Then the white suspension was cooled to −78° C. To this mixture was added Ti(i-PrO)₄ (4.63 g, 16.3 mmol, 4.81 mL, 1.5 eq) slowly during a period of 5 min and stirred at −78° C. for 10 min. It still was a white suspension. EtMgBr (3 M, in Et₂O, 7.96 mL, 2.2 eq) was added dropwise to maintain the temperature between −78° C. and −75° C. over 10 min under a N₂ atmosphere. The color of the mixture turned to brown after the addition was complete. The resulting mixture was stirred at the same temperature for 10 min and then warmed to room temperature (between 15-20° C.) slowly over 1.5 h. The mixture turned to black. To the mixture was added BF₃·Et₂O (3.08 g, 21.7 mmol, 2.68 mL, 2.0 eq) in portions at the same temperature with no obvious temperature change. The resulting mixture was stirred at room temperature for another 1 h. LCMS showed some SM remained and 33% desired product was detected. The reaction mixture was poured into a mixture of HCl (1 M aqueous) (100 mL) and MTBE (100 mL) and extracted with MTBE (80 mL×2). The MTBE organic layers were discarded. The aqueous layer was basified to pH 8 by using NaOH (2 M aqueous), and a precipitate was formed. The mixture was filtered through a pad of Celite and the slurry was washed with DCM several times. The combined filtrate was extracted with DCM (100 mL×3). The combined organic layers were dried over Na₂SO₄, filtered, and concentrated under vacuum to give a residue which was purified by flash silica gel chromatography using a gradient of EtOAc/petroleum ether from 0/1 to 7/3. 1-(7-Methoxyquinolin-5-yl)cyclopropanamine (1.00 g, 4.67 mmol, 43% yield) was obtained as a brown solid. M+H⁺=215.0 (LCMS); ¹H NMR (400 MHZ, CDCl₃) δ 8.79 (dd, J=1.6, 4.3 Hz, 1H), 8.61 (dd, J=0.9, 8.4 Hz, 1H), 7.31-7.27 (m, 1H), 7.21 (s, 1H), 7.18 (d, J=2.5 Hz, 1H), 3.89 (s, 3H), 1.15-1.08 (m, 2H), 0.97-0.92 (m, 2H).

### Step 3: 5-(2-(Dimethylamino)ethoxy)-N-(1-(7-methoxyquinolin-5-yl)cyclopropyl)-2-methylbenzamide (Compound 434)

To a solution of 5-(2-(dimethylamino)ethoxy)-2-methylbenzoic acid (400 mg, 1.79 mmol, 1.0 eq) and 1-(7-methoxyquinolin-5-yl)cyclopropanamine (500 mg, 2.34 mmol, 1.3 eq) in DMF (20 mL) were added HATU (1.70 g, 4.48 mmol, 2.5 eq), and DIEA (694 mg, 5.38 mmol, 936 μL, 3.0 eq). The mixture was stirred at 20° C. for 3 h. LCMS indicated that the starting material was completely consumed, and the desired mass was detected. The reaction mixture was poured into H₂O (30 mL) and extracted with EtOAc (20 mL×3). The combined organic layers were dried over Na₂SO₄, filtered, and concentrated under vacuum to give a residue which was purified by preparative TLC (DCM/MeOH=5/1, R_{f}=0.35) to give the free base product. The free base product was dealt with acetonitrile (1.0 mL), followed by HCl (0.40% aqueous, 2.0 mL) and lyophilized to give 5-(2-(dimethylamino)ethoxy)-N-(1-(7-methoxyquinolin-5-yl)cyclopropyl)-2-methylbenzamide (122 mg, 254 μmol, 14% yield, HCl salt) as a white solid. M+H⁺=420.1 (LCMS); ¹H NMR (400 MHZ, DMSO-d₆) δ 9.12 (s, 1H), 8.95 (d, J=8.4 Hz, 1H), 8.82 (dd, J=1.6, 4.2 Hz, 1H), 7.47 (d, J=2.6 Hz, 1H), 7.42 (dd, J=4.2, 8.4 Hz, 1H), 7.31 (d, J=2.5 Hz, 1H), 7.05 (d, J=8.4 Hz, 1H), 6.86 (dd, J=2.7, 8.4 Hz, 1H), 6.64 (d, J=2.6 Hz, 1H), 4.05 (br t, J=5.4 Hz, 2H), 3.92 (s, 3H), 2.88 (br d, J=8.9 Hz, 2H), 2.39 (br s, 6H), 1.95 (s, 3H), 1.37-1.31 (m, 2H), 1.22-1.16 (m, 2H).

### Example 334: (S)-5-(2-Aminopropoxy)-N-(1-(7-methoxyquinolin-5-yl)cyclopropyl)-2-methylbenzamide (Compound 530)

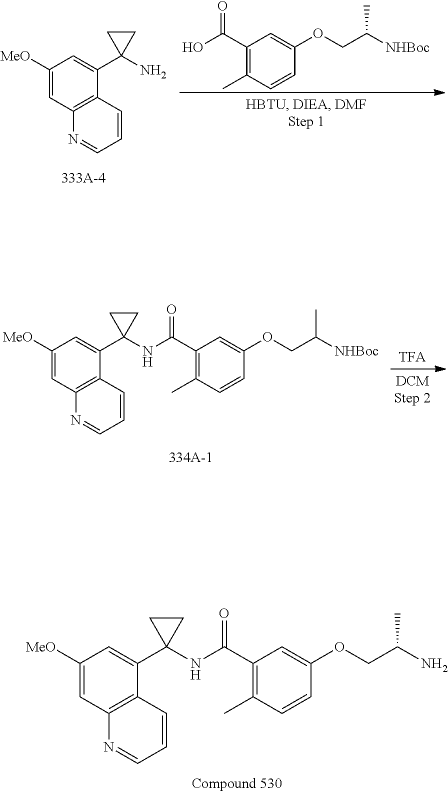 [SMILES: COc1cc(C2(N)CC2)c2cccnc2c1 ; Cc1ccc(OC[C@H](C)NC(=O)OC(C)(C)C)cc1C(=O)O ; COc1cc(C2(NC(=O)c3cc(OCC(C)NC(=O)OC(C)(C)C)ccc3C)CC2)c2cccnc2c1 ; COc1cc(C2(NC(=O)c3cc(OC[C@H](C)N)ccc3C)CC2)c2cccnc2c1]

### Step 1: tert-Butyl(1-(3-((1-(7-methoxyquinolin-5-yl)cyclopropyl)carbamoyl)-4-methylphenoxy)propan-2-yl)carbamate (334A-1)

To a solution of 1-(7-methoxyquinolin-5-yl)cyclopropanamine (20.0 mg, 93.3 μmol, 1.0 eq) and(S)-5-(2-((tert-butoxycarbonyl)amino)propoxy)-2-methylbenzoic acid (28.9 mg, 93.4 μmol, 1.0 eq) in DMF (1.0 mL) were added HBTU (δ8.5 mg, 233 μmol, 2.5 eq) and DIEA (48.3 mg, 373 μmol, 65.0 μL, 4.0 eq). The mixture was stirred at 20° C. for 16 h. LCMS indicated that the starting material was completely consumed, and the desired mass was detected. The mixture was treated with water (10 mL) and extracted with EtOAc (5.0 mL×5). The combined organic layers were dried over Na₂SO₄, filtered, and concentrated under vacuum to give a curde product tert-butyl(1-(3-((1-(7-methoxyquinolin-5-yl)cyclopropyl)carbamoyl)-4-methylphenoxy)propan-2-yl)carbamate (120 mg) as a colorless oil. M+H⁺=506.3 (LCMS).

### Step 2: (S)-5-(2-Aminopropoxy)-N-(1-(7-methoxyquinolin-5-yl)cyclopropyl)-2-methylbenzamide (Compound 530)

To a solution of tert-butyl(1-(3-((1-(7-methoxyquinolin-5-yl)cyclopropyl)carbamoyl)-4-methylphenoxy)propan-2-yl)carbamate (120 mg, 237 μmol, 1.0 eq) in DCM (1.0 mL) was added TFA (1.62 g, 14.3 mmol, 1.05 mL, 60 eq). The mixture was stirred at 20° C. for 2 h. LCMS indicated that the starting material was completely consumed, and the desired mass was detected. The reaction mixture was concentrated under vacuum to give a residue which was purified by preparative HPLC (Phenomenex Luna C18 column (75×30 mm, 3 μm); flow rate: 25 mL/min; gradient: 1%-30% B over 8 min; mobile phase A: 0.1% aqueous TFA, mobile phase B: acetonitrile). (S)-5-(2-Aminopropoxy)-N-(1-(7-methoxyquinolin-5-yl)cyclopropyl)-2-methylbenzamide (4.04 mg, 7.54 μmol, 3% yield, TFA salt) was obtained as a pale yellow gum. M+H⁺=406.2 (LCMS); ¹H NMR (400 MHZ, DMSO-d₆) δ 9.16 (s, 1H), 9.10 (br d, J=8.0 Hz, 1H), 8.92 (d, J=3.5 Hz, 1H), 7.91 (br s, 2H), 7.60-7.53 (m, 2H), 7.36 (d, J=2.4 Hz, 1H), 7.09 (d, J=8.5 Hz, 1H), 6.90 (dd, J=2.7, 8.4 Hz, 1H), 6.68 (d, J=2.6 Hz, 1H), 4.03 (dd, J=3.9, 10.3 Hz, 1H), 3.95 (s, 3H), 3.85 (dd, J=7.2, 10.3 Hz, 1H), 3.60-3.51 (m, 1H), 1.96 (s, 3H), 1.38-1.32 (m, 2H), 1.22 (d, J=6.6 Hz, 5H).

### Example 335: N-(1-(7-Methoxyquinolin-5-yl)cyclopropyl)-2-methyl-5-(2-(methylamino)

ethoxy)benzamide (Compound 526)

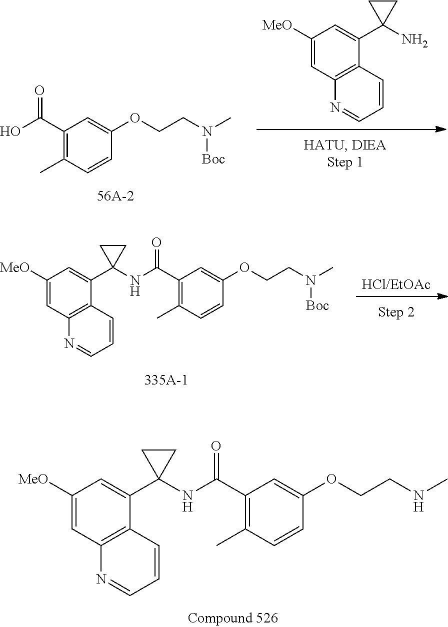 [SMILES: Cc1ccc(OCCN(C)C(=O)OC(C)(C)C)cc1C(=O)O ; COc1cc(C2(N)CC2)c2cccnc2c1 ; COc1cc(C2(NC(=O)c3cc(OCCN(C)C(=O)OC(C)(C)C)ccc3C)CC2)c2cccnc2c1 ; "HCl/EtOAc" ; CNCCOc1ccc(C)c(C(=O)NC2(c3cc(OC)cc4ncccc34)CC2)c1]

### Step 1: tert-Butyl(2-(3-((1-(7-methoxyquinolin-5-yl)cyclopropyl)carbamoyl)-4-methyl phenoxy)ethyl)(methyl)carbamate (335A-1)

To a solution of 5-(2-((tert-butoxycarbonyl)(methyl)amino)ethoxy)-2-methylbenzoic acid (150 mg, 485 μmol, 1.0 eq) in DMF (6.0 mL) were added 1-(7-methoxyquinolin-5-yl)cyclopropanamine (260 mg, 1.21 mmol, 2.5 eq), DIEA (188 mg, 1.45 mmol, 254 μL, 3.0 eq) and HATU (461 mg, 1.21 mmol, 2.5 eq). The mixture was stirred at 20° C. for 16 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/0. tert-Butyl(2-(3-((1-(7-methoxyquinolin-5-yl)cyclopropyl)carbamoyl)-4-methylphenoxy)ethyl)(methyl)carbamate (200 mg, 396 μmol, 82% yield) was obtained as a brown oil. M+H⁺=506.2 (LCMS).

### Step 2: N-(1-(7-Methoxyquinolin-5-yl)cyclopropyl)-2-methyl-5-(2-(methylamino)ethoxy)benzamide (Compound 526)

To a solution of tert-butyl(2-(3-((1-(7-methoxyquinolin-5-yl)cyclopropyl)carbamoyl)-4-methylphenoxy)ethyl)(methyl)carbamate (200 mg, 396 μmol, 1.0 eq) in EtOAc (3.0 mL) was added HCl/EtOAc (4 M, 3.0 mL). The resulting mixture was stirred at 25° C. for 30 min. LCMS indicated that the starting material was completely consumed, and the desired mass was detected. The reaction mixture was concentrated under vacuum to give a residue which was purified by preparative HPLC (Phenomenex Luna C18 column (80×30 mm, 3 μm); flow rate: 25 mL/min; gradient: 1%-40% B over 8 min; mobile phase A: 0.04% aqueous HCl, mobile phase B: acetonitrile). N-(1-(7-Methoxyquinolin-5-yl)cyclopropyl)-2-methyl-5-(2-(methylamino)ethoxy)benzamide (110 mg, 243 μmol, 61% yield, HCl salt) was obtained as a yellow solid. M+H⁺=406.1 (LCMS); ¹H NMR (400 MHZ, DMSO-d₆) δ 9.64 (br d, J=8.4 Hz, 1H), 9.36 (s, 1H), 9.22-9.05 (m, 3H), 7.94 (dd, J=5.3, 8.4 Hz, 1H), 7.71 (d, J=2.4 Hz, 1H), 7.66 (d, J=2.1 Hz, 1H), 7.09 (d, J=8.5 Hz, 1H), 6.91 (dd, J=2.6, 8.4 Hz, 1H), 6.74 (d, J=2.6 Hz, 1H), 4.19 (t, J=5.1 Hz, 2H), 4.01 (s, 3H), 3.24 (quin, J=5.4 Hz, 2H), 2.57 (t, J=5.4 Hz, 3H), 1.97 (s, 3H), 1.44-1.38 (m, 2H), 1.33-1.28 (m, 2H).

### Example 336: (S)—N-(1-(7-Methoxyquinolin-5-yl)cyclopropyl)-2-methyl-5-(2-(methylamino)propoxy)benzamide (Compound 560)

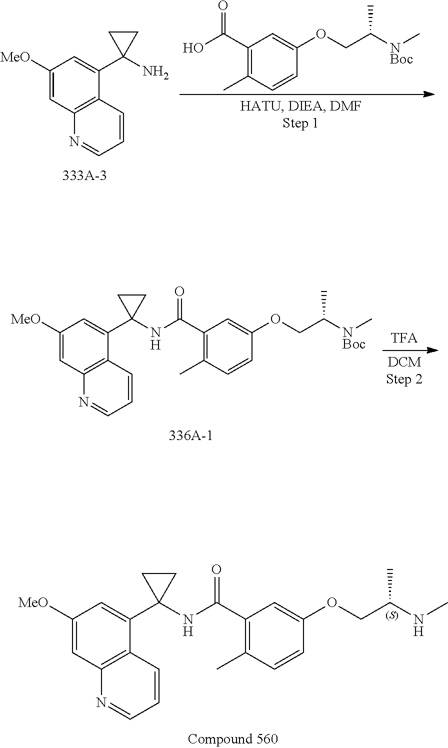

### Step 1: (S)-tert-Butyl(1-(3-((1-(7-methoxyquinolin-5-yl)cyclopropyl)carbamoyl)-4-methylphenoxy)propan-2-yl)(methyl)carbamate (336A-1)

To a solution of 1-(7-methoxyquinolin-5-yl)cyclopropanamine (30.0 mg, 140 μmol, 1.0 eq) and(S)-5-(2-((tert-butoxycarbonyl)(methyl)amino)propoxy)-2-methylbenzoic acid (45.3 mg, 140 μmol, 1.0 eq) in DMF (1.0 mL) were added DIEA (72.4 mg, 560 μmol, 97.5 μL, 4.0 eq) and HATU (133 mg, 350 μmol, 2.5 eq). The mixture was stirred at 20° C. for 2 h. LCMS indicated that the starting material was completely consumed. The mixture was poured into H₂O (5.0 mL) and extracted with EtOAc (5.0 mL×3). The combined organic layers were dried over Na₂SO₄, filtered, and concentrated under vacuum to give the crude product(S)-tert-butyl(1-(3-((1-(7-methoxyquinolin-5-yl)cyclopropyl)carbamoyl)-4-methylphenoxy)propan-2-yl)(methyl)carbamate (80.0 mg) as a brown liquid. M+H⁺=520.3 (LCMS).

### Step 2: (S)—N-(1-(7-Methoxyquinolin-5-yl)cyclopropyl)-2-methyl-5-(2-(methylamino)propoxy)benzamide (Compound 560)

To a solution of(S)-tert-butyl(1-(3-((1-(7-methoxyquinolin-5-yl)cyclopropyl)carbamoyl)-4-methylphenoxy)propan-2-yl)(methyl)carbamate (80.0 mg, 154 μmol, 1.0 eq) in EtOAc (1.0 mL) was added HCl/EtOAc (4 M, 1.0 mL). The mixture was stirred at 20° C. for 1 h. LCMS indicated that the starting material was completely consumed. The mixture was concentrated under vacuum to give a residue which was purified by preparative HPLC (Phenomenex Luna C18 column (80×30 mm, 3 μm); flow rate: 25 mL/min; gradient: 1%-25% B over 8 min; mobile phase A: 0.1% aqueous TFA, mobile phase B: acetonitrile). (S)—N-(1-(7-Methoxyquinolin-5-yl)cyclopropyl)-2-methyl-5-(2-(methylamino)propoxy)benzamide (15.8 mg, 33.5 μmol, 21% yield, HCl salt) was obtained as a pale yellow gum. M+H⁺=420.2 (LCMS); ¹H NMR (400 MHZ, DMSO-d₆) δ 9.73 (d, J=8.50 Hz, 1H), 9.43-9.36 (m, 1H), 9.34-9.25 (m, 1H), 9.20 (d, J=4.50 Hz, 1H), 9.17-9.06 (m, 1H), 8.00 (dd, J=8.38, 5.50 Hz, 1H), 7.77-7.71 (m, 2H), 7.09 (d, J=8.38 Hz, 1H), 6.92 (dd, J=8.38, 2.63 Hz, 1H), 6.76 (d, J=2.63 Hz, 1H), 4.18-4.13 (m, 1H), 4.11-4.07 (m, 1H), 4.02 (s, 3H), 3.56-3.45 (m, 1H), 2.56-2.52 (m, 3H), 1.96 (s, 3H), 1.42 (br s, 2H), 1.33-1.27 (m, 5H).

### Example 337: (S)-5-(2-(Dimethylamino)propoxy)-N-(1-(7-methoxyquinolin-5-yl)cyclopropyl)-2-methylbenzamide (Compound 575)

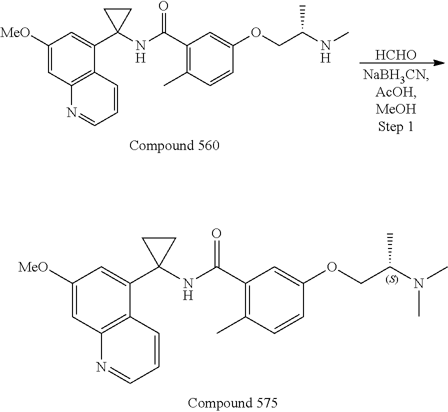

### Step 1: (S)-5-(2-(Dimethylamino)propoxy)-N-(1-(7-methoxyquinolin-5-yl)cyclopropyl)-2-methylbenzamide (Compound 575)

To a solution of(S)—N-(1-(7-methoxyquinolin-5-yl)cyclopropyl)-2-methyl-5-(2-(methylamino)propoxy)benzamide (176 mg, 421 μmol, 1.0 eq) in MeOH (2.0 mL) was added formaldehyde (68.4 mg, 842 μmol, 62.7 μL, 37% purity in water, 2.0 eq). The resulting mixture was adjusted to pH 6 with a small amount of AcOH. The resulting mixture was stirred at 20° C. for 30 min, then NaBH₃CN (79.4 mg, 1.26 mmol, 3.0 eq) was added. The reaction mixture was stirred at 20° C. for another 16 h. LCMS indicated that the starting material was completely consumed, and the desired mass was detected. The reaction mixture was concentrated under vacuum and the residue was purified by preparative HPLC (Phenomenex Gemini C18 column (80×30 mm, 3 μm); flow rate: 60 mL/min; gradient: 1%-30% B over 8 min; mobile phase A: 0.04% aqueous HCl, mobile phase B: acetonitrile). (S)-5-(2-(Dimethylamino)propoxy)-N-(1-(7-methoxyquinolin-5-yl)cyclopropyl)-2-methylbenzamide (39.1 mg, 82.0 μmol, 19% yield) was obtained as a pale yellow solid. M+H⁺=434.3 (LCMS); ¹H NMR (400 MHZ, DMSO-d₆) δ 10.62 (br d, J=1.3 Hz, 1H), 9.73 (d, J=8.1 Hz, 1H), 9.39 (s, 1H), 9.19 (dd, J=1.1, 5.4 Hz, 1H), 8.05-7.93 (m, 1H), 7.79-7.65 (m, 2H), 7.09 (d, J=8.5 Hz, 1H), 6.93 (dd, J=2.8, 8.4 Hz, 1H), 6.79 (d, J=2.8 Hz, 1H), 4.21-4.18 (m, 2H), 4.02 (s, 3H), 3.74-3.68 (m, 1H), 2.73 (dd, J=5.0, 8.9 Hz, 6H), 1.96 (s, 3H), 1.47-1.40 (m, 2H), 1.31 (d, J=6.8 Hz, 5H).

### Example 338: (S)-5-(Azetidin-2-ylmethoxy)-N-(1-(7-methoxyquinolin-5-yl)cyclopropyl)-2-methylbenzamide (Compound 511)

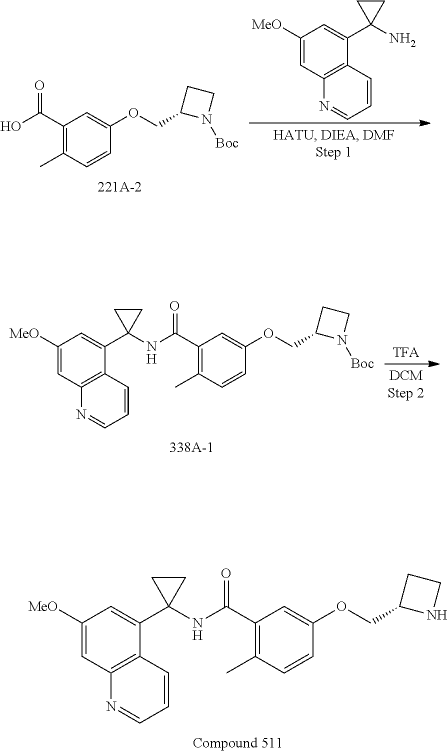 [SMILES: Cc1ccc(OC[C@@H]2CCN2C(=O)OC(C)(C)C)cc1C(=O)O ; COc1cc(C2(N)CC2)c2cccnc2c1 ; COc1cc(C2(NC(=O)c3cc(OC[C@@H]4CCN4C(=O)OC(C)(C)C)ccc3C)CC2)c2cccnc2c1 ; COc1cc(C2(NC(=O)c3cc(OC[C@@H]4CCN4)ccc3C)CC2)c2cccnc2c1]

### Step 1: (S)-tert-Butyl 2-((3-((1-(7-methoxyquinolin-5-yl)cyclopropyl)carbamoyl)-4-methylphenoxy)methyl)azetidine-1-carboxylate (338A-1)

To a solution of(S)-5-((1-(tert-butoxycarbonyl)azetidin-2-yl)methoxy)-2-methylbenzoic acid (300 mg, 934 μmol, 1.0 eq) in DMF (10 mL) were added 1-(7-methoxyquinolin-5-yl)cyclopropanamine (300 mg, 1.40 mmol, 1.5 eq), DIEA (362 mg, 2.80 mmol, 488 μL, 3.0 eq) and HATU (δ87 mg, 2.33 mmol, 2.5 eq). The mixture was stirred at 20° C. for 16 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 4/5. (S)-tert-Butyl 2-((3-((1-(7-methoxyquinolin-5-yl)cyclopropyl)carbamoyl)-4-methylphenoxy)methyl)azetidine-1-carboxylate (400 mg, 773 μmol, 83% yield) was obtained as a yellow solid. M+H⁺=518.2 (LCMS).

### Step 2: (S)-5-(Azetidin-2-ylmethoxy)-N-(1-(7-methoxyquinolin-5-yl)cyclopropyl)-2-methylbenzamide (Compound 511)

To a solution of(S)-tert-butyl 2-((3-((1-(7-methoxyquinolin-5-yl)cyclopropyl)carbamoyl)-4-methylphenoxy)methyl)azetidine-1-carboxylate (400 mg, 773 μmol, 1.0 eq) in DCM (4.0 mL) was added TFA (1.0 mL). The resulting mixture was stirred at 25° C. for 30 min. LCMS indicated that the starting material was completely consumed, and the desired mass was detected. The reaction mixture was concentrated under vacuum at 30° C. to give a residue which was purified by preparative HPLC (Phenomenex Luna C18 column (80×30 mm, 3 μm); flow rate: 25 mL/min; gradient: 5%-45% B over 8 min; mobile phase A: 0.04% aqueous HCl, mobile phase B: acetonitrile). (S)-5-(Azetidin-2-ylmethoxy)-N-(1-(7-methoxyquinolin-5-yl)cyclopropyl)-2-methylbenzamide (48.9 mg, 116 μmol, 15% yield, HCl salt) was obtained as a yellow solid. M+H⁺=418.1 (LCMS); ¹H NMR (400 MHZ, DMSO-d₆) δ 9.35 (br d, J=8.4 Hz, 1H), 9.28-9.13 (m, 2H), 9.03 (br d, J=3.8 Hz, 2H), 7.73 (dd, J=4.9, 8.4 Hz, 1H), 7.62 (d, J=2.5 Hz, 1H), 7.48 (d, J=2.3 Hz, 1H), 7.10 (d, J=8.6 Hz, 1H), 6.92 (dd, J=2.8, 8.4 Hz, 1H), 6.73 (d, J=2.8 Hz, 1H), 4.65 (br d, J=6.4 Hz, 1H), 4.28 (dd, J=7.4, 11.1 Hz, 1H), 4.14 (dd, J=3.3, 11.1 Hz, 1H), 3.98 (s, 3H), 3.95-3.76 (m, 2H), 2.48-2.27 (m, 2H), 1.97 (s, 3H), 1.43-1.34 (m, 2H), 1.30-1.22 (m, 2H).

### Example 339: (S)—N-(1-(7-Methoxyquinolin-5-yl)cyclopropyl)-2-methyl-5-((1-methylazetidin-2-yl)methoxy)benzamide (Compound 527)

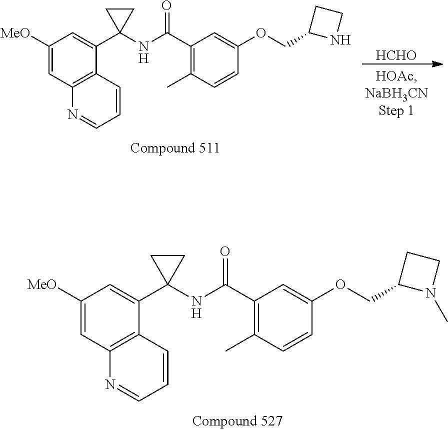

### Step 1: (S)—N-(1-(7-Methoxyquinolin-5-yl)cyclopropyl)-2-methyl-5-((1-methylazetidin-2-yl)methoxy)benzamide (Compound 527)

To a solution of(S)-5-(azetidin-2-ylmethoxy)-N-(1-(7-methoxyquinolin-5-yl)cyclopropyl)-2-methylbenzamide (250 mg, 599 μmol, 1.0 eq) in MeOH (10 mL) was added TEA (50.0 μL), followed by formaldehyde (36.0 mg, 1.20 mmol, 32.9 μL, 37% purity in water, 2.0 eq). The resulting mixture was adjusted to pH 6 with a small amount of AcOH. The mixture was stirred at 20° C. for 30 min, then NaBH₃CN (75.0 mg, 1.20 mmol, 2.0 eq) was added. The resulting reaction mixture was stirred at 20° C. for another 16 h. LCMS indicated that the starting material was completely consumed, and the desired mass was detected. The mixture was treated with H₂O (10 mL) and extracted with DCM (10 mL×5). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 5%-40% B over 8 min; mobile phase A: 0.04% aqueous HCl, mobile phase B: acetonitrile). (S)—N-(1-(7-Methoxyquinolin-5-yl)cyclopropyl)-2-methyl-5-((1-methylazetidin-2-yl)methoxy)benzamide (87.2 mg, 183 μmol, 31% yield, HCl salt) was obtained as a yellow solid. M+H⁺=432.1 (LCMS); ¹H NMR (400 MHZ, DMSO-d₆) δ 10.83-10.68 (m, 1H), 9.59-9.48 (m, 1H), 9.31 (s, 1H), 9.12 (br d, J=4.9 Hz, 1H), 7.87 (br s, 1H), 7.69 (s, 1H), 7.58 (br s, 1H), 7.15-7.06 (m, 1H), 7.00-6.89 (m, 1H), 6.83-6.71 (m, 1H), 4.68-4.58 (m, 1H), 4.38 (br dd, J=8.4, 10.8 Hz, 1H), 4.22 (dd, J=3.2, 11.2 Hz, 1H), 4.01 (s, 3H), 3.85 (br dd, J=6.7, 9.3 Hz, 2H), 2.81 (d, J=5.0 Hz, 3H), 2.45-2.22 (m, 2H), 1.96 (s, 3H), 1.41 (br s, 2H), 1.30 (br s, 2H).

### Example 340: (S)—N-(1-(7-Methoxyquinolin-5-yl)cyclopropyl)-2-methyl-5-(pyrrolidin-2-ylmethoxy)benzamide (Compound 462)

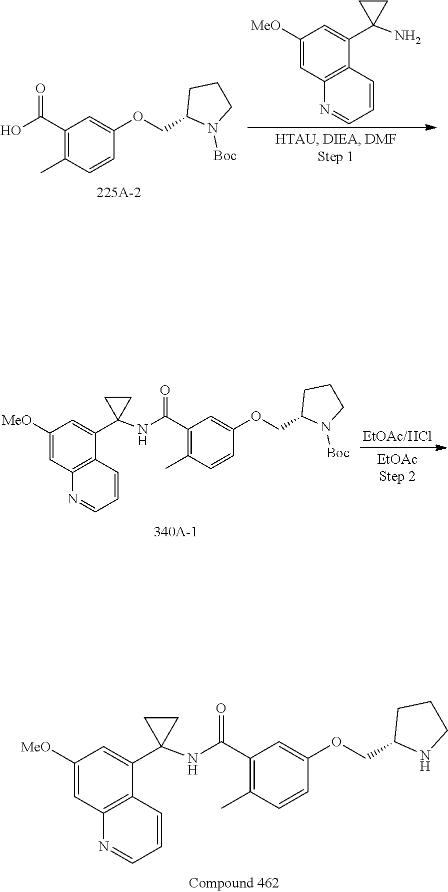 [SMILES: Cc1ccc(OC[C@@H]2CCCN2C(=O)OC(C)(C)C)cc1C(=O)O ; COc1cc(C2(N)CC2)c2cccnc2c1 ; COc1cc(C2(NC(=O)c3cc(OC[C@@H]4CCCN4C(=O)OC(C)(C)C)ccc3C)CC2)c2cccnc2c1 ; COc1cc(C2(NC(=O)c3cc(OC[C@@H]4CCCN4)ccc3C)CC2)c2cccnc2c1]

### Step 1: (S)-tert-Butyl 2-((3-((1-(7-methoxyquinolin-5-yl)cyclopropyl)carbamoyl)-4-methylphenoxy)methyl)pyrrolidine-1-carboxylate (340A-1)

To a solution of 1-(7-methoxyquinolin-5-yl)cyclopropanamine (192 mg, 895 μmol, 1.5 eq) and(S)-5-((1-(tert-butoxycarbonyl)pyrrolidin-2-yl)methoxy)-2-methylbenzoic acid (200 mg, 596 μmol, 1.0 eq) in DMF (10 mL) were added DIEA (231 mg, 1.79 mmol, 312 μL, 3.0 eq) and HATU (567 mg, 1.49 mmol, 2.5 eq). The resulting mixture was stirred at 20° C. for 16 h. LCMS indicated that the starting material was completely consumed, and the desired mass was detected. The mixture was poured into H₂O (15 mL) and extracted with EtOAc (6.0 mL×3). The combined organic layers were dried over Na₂SO₄, filtered, and concentrated under vacuum to give a residue which was purified by flash silica gel chromatography using a gradient of EtOAc/petroleum ether from 0/1 to 3/5. (S)-tert-Butyl 2-((3-((1-(7-methoxyquinolin-5-yl)cyclopropyl)carbamoyl)-4-methylphenoxy)methyl)pyrrolidine-1-carboxylate (254 mg, 478 μmol, 40% yield) was obtained as a yellow oil. M+H⁺=532.3 (LCMS).

### Step 2: (S)—N-(1-(7-Methoxyquinolin-5-yl)cyclopropyl)-2-methyl-5-(pyrrolidin-2-ylmethoxy)benzamide (Compound 462)

To a solution of(S)-tert-butyl 2-((3-((1-(7-methoxyquinolin-5-yl)cyclopropyl)carbamoyl)-4-methylphenoxy)methyl)pyrrolidine-1-carboxylate (254 mg, 478 μmol, 1.0 eq) in EtOAc (1.0 mL) was added HCl/EtOAc (4 M, 20 mL). The mixture was stirred at 20° C. for 1 h. LCMS indicated that the starting material was completely consumed, and the desired mass was detected. The mixture was concentrated under vacuum at 20° C. to give a residue which was purified by preparative HPLC (Phenomenex Luna column (80×30 mm, 3 μm); flow rate: 25 mL/min; gradient: 1%-40% B over 8 min; mobile phase A: 0.04% aqueous HCl, mobile phase B: acetonitrile). (S)—N-(1-(7-Methoxyquinolin-5-yl)cyclopropyl)-2-methyl-5-(pyrrolidin-2-ylmethoxy)benzamide (123 mg, 264 μmol, 55% yield, HCl salt) was obtained as a yellow solid. M+H⁺=432.1 (LCMS); ¹H NMR (400 MHZ, DMSO-d₆) δ 9.70 (d, J=8.4 Hz, 2H), 9.36 (s, 1H), 9.14 (d, J=5.3 Hz, 2H), 7.97 (dd, J=5.5, 8.5 Hz, 1H), 7.74 (d, J=2.4 Hz, 2H), 7.08 (d, J=8.5 Hz, 1H), 6.90 (dd, J=2.6, 8.4 Hz, 1H), 6.72 (d, J=2.6 Hz, 1H), 4.22-4.13 (m, 2H), 4.10-3.97 (m, 3H), 3.86-3.79 (m, 1H), 3.25-3.11 (m, 2H), 2.15-2.03 (m, 1H), 2.02-1.81 (m, 5H), 1.68 (qd, J=8.2, 12.7 Hz, 1H), 1.42 (br s, 2H), 1.30 (br s, 2H).

### Example 341: (S)—N-(1-(7-Methoxyquinolin-5-yl)cyclopropyl)-2-methyl-5-((1-methylpyrrolidin-2-yl)methoxy)benzamide (Compound 528)

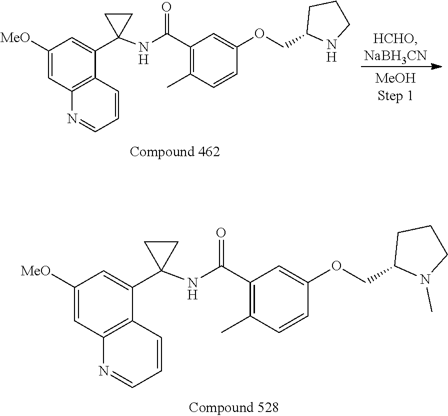

### Step 1: (S)—N-(1-(7-Methoxyquinolin-5-yl)cyclopropyl)-2-methyl-5-((1-methylpyrrolidin-2-yl)methoxy)benzamide (Compound 528)

To a solution of(S)—N-(1-(7-methoxyquinolin-5-yl)cyclopropyl)-2-methyl-5-(pyrrolidin-2-ylmethoxy)benzamide (70.0 mg, 150 μmol, 1.0 eq, HCl salt) in MeOH (2.5 mL) was added TEA (0.30 mL), followed by formaldehyde (8.98 mg, 299 μmol, 8.24 μl, 37% purity in water, 2.0 eq). The resulting mixture was adjusted to pH 6 with a small amount of AcOH. The mixture was stirred at 20° C. for 30 min and NaBH₃CN (18.8 mg, 299 μmol, 2.0 eq) was added. The reaction mixture was stirred at 20° C. for 15 h. LCMS indicated that the starting material was completely consumed, and the desired mass was detected. The reaction mixture was poured into H₂O (5.0 mL) and extracted with EtOAc (3.0 mL×2). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 1%-15% B over 8 min; mobile phase A: 0.04% aqueous HCl, mobile phase B: acetonitrile). (S)—N-(1-(7-Methoxyquinolin-5-yl)cyclopropyl)-2-methyl-5-(pyrrolidin-2-ylmethoxy)benzamide (22.1 mg, 45.9 μmol, 31% yield, HCl salt) was obtained as a yellow solid. M+H⁺=446.1 (LCMS); ¹H NMR (400 MHZ, DMSO-d₆) δ 10.94 (br s, 1H), 9.72 (d, J=8.5 Hz, 1H), 9.38 (s, 1H), 9.19 (d, J=4.8 Hz, 1H), 7.99 (dd, J=5.4, 8.4 Hz, 1H), 7.72 (dd, J=2.3, 16.4 Hz, 2H), 7.09 (d, J=8.4 Hz, 1H), 6.92 (dd, J=2.6, 8.4 Hz, 1H), 6.77 (d, J=2.6 Hz, 1H), 4.38-4.30 (m, 1H), 4.28-4.22 (m, 1H), 4.02 (s, 3H), 3.74 (dt, J=3.3, 8.0 Hz, 1H), 3.61-3.45 (m, 1H), 3.15-3.01 (m, 1H), 2.88 (d, J=4.8 Hz, 3H), 2.28-2.14 (m, 1H), 2.03-1.87 (m, 5H), 1.83-1.69 (m, 1H), 1.42 (br s, 2H), 1.32 (br s, 2H).

### Example 342: (S)—N-(1-(7-Methoxyquinolin-5-yl)cyclopropyl)-2-methyl-5-((1-methyl

piperidin-2-yl)methoxy)benzamide (Compound 550)

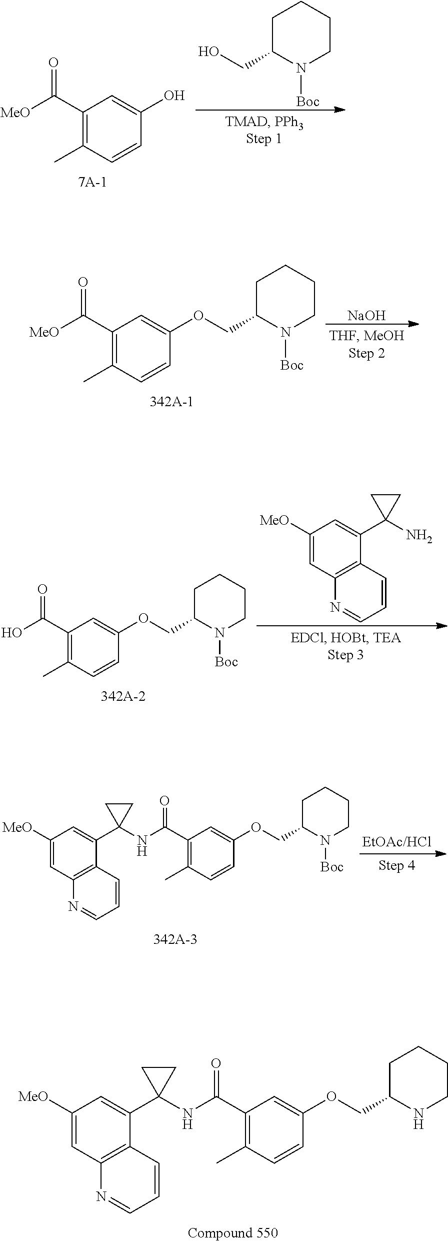

### Step 1: (S)-tert-Butyl 2-((3-(methoxycarbonyl)-4-methylphenoxy)methyl)piperidine-1-carboxylate (342A-1)

To a solution of methyl 5-hydroxy-2-methylbenzoate (300 mg, 1.81 mmol, 1.0 eq) and(S)-tert-butyl 2-(hydroxymethyl)piperidine-1-carboxylate (583 mg, 2.70 mmol, 1.5 eq) in toluene (15 mL) were added TMAD (930 mg, 5.42 mmol, 3.0 eq), PPh₃ (1.40 g, 5.42 mmol, 3.0 eq). The resulting mixture was stirred at 120° C. for 16 h under a N₂ atmosphere. LCMS indicated that the starting material was completely consumed, and the desired compound was detected. The reaction mixture was allowed to cool to room temperature, poured into water (10 mL) and extracted with EtOAc (10 mL×3). The combined organic layers were dried over Na₂SO₄. filtered, and concentrated under vacuum to give a residue which was purified by flash silica gel chromatography using a gradient of EtOAc/petroleum ether from 0/1 to 1/10. (S)-tert-Butyl 2-((3-(methoxycarbonyl)-4-methylphenoxy)methyl)piperidine-1-carboxylate (470 mg, 1.16 mmol, 72% yield) was obtained as a yellow solid. M−100+H⁺=264.1 (LCMS); ¹H NMR (400 MHZ, DMSO-d₆) δ 7.34 (d, J=2.8 Hz, 1H), 7.23 (d, J=8.5 Hz, 1H), 7.09 (dd, J=2.8, 8.4 Hz, 1H), 4.48-4.38 (m, 1H), 4.17-4.05 (m, 2H), 3.92-3.83 (m, 1H), 3.81 (s, 3H), 2.84 (br t, J=12.4 Hz, 1H), 2.42 (s, 3H), 1.78 (br d, J=7.4 Hz, 1H), 1.64-1.48 (m, 4H), 1.35 (s, 9H), 1.32-1.25 (m, 1H).

### Step 2: (S)-5-((1-(tert-Butoxycarbonyl) piperidin-2-yl)methoxy)-2-methylbenzoic acid (342A-2)

To a solution of(S)-tert-butyl 2-((3-(methoxycarbonyl)-4-methylphenoxy)methyl)piperidine-1-carboxylate (470 mg, 1.16 mmol, 1.0 eq) in a mixture of THF (3.0 mL) and MeOH (1.0 mL) was added NaOH (2 M aqueous, 2.33 mL, 4.0 eq). The mixture was stirred at 70° C. for 16 h. LCMS indicated that the starting material was completely consumed, and the desired mass was detected. The reaction mixture was allowed to cool to room temperature, poured into H₂O (10 mL) and washed with EtOAc (10 mL×3). The aqueous layer was acidified to pH 3 with HCl (1 M aqueous). The product was extracted with EtOAc (10 mL×3). The combined organic layers were dried over Na₂SO₄ filtered, and concentrated under vacuum to give the crude product(S)-5-((1-(tert-Butoxycarbonyl) piperidin-2-yl)methoxy)-2-methylbenzoic acid (260 mg) was obtained as a white solid, which was used in the next step without any further purification. M−100+H⁺=250.1 (LCMS).

### Step 3: (S)-tert-Butyl 2-((3-((1-(7-methoxyquinolin-5-yl)cyclopropyl)carbamoyl)-4-methylphenoxy)methyl)piperidine-1-carboxylate (342A-3)

To a solution of(S)-5-((1-(tert-butoxycarbonyl) piperidin-2-yl)methoxy)-2-methylbenzoic acid (230 mg, 658 μmol, 1.0 eq) and 1-(7-methoxyquinolin-5-yl)cyclopropanamine (141 mg, 658 μmol, 1.0 eq) in DCM (1.0 mL) were added EDCI (189 mg, 987 μmol, 1.5 eq), HOBt (133 mg, 987 μmol, 1.5 eq) and TEA (200 mg, 1.97 mmol, 275 μL, 3.0 eq). The resulting mixture was stirred at 20° C. for 1 h. LCMS indicated that the starting material was completely consumed, and the desired compound was detected. The mixture was poured into H₂O (10 mL) and extracted with DCM (10 mL×3). The combined organic layers were dried over Na₂SO₄, filtered, and concentrated under vacuum to give a residue which was purified by preparative TLC (EtOAc/petroleum ether=1/0, R_{f}=0.5). (S)-tert-Butyl2-((3-((1-(7-methoxyquinolin-5-yl)cyclopropyl)carbamoyl)-4-methyl phenoxy)methyl)piperidine-1-carboxylate (200 mg, 367 μmol, 56% yield) was obtained as a yellow oil. M+H⁺=546.3 (LCMS).

### Step 4: (S)—N-(1-(7-Methoxyquinolin-5-yl)cyclopropyl)-2-methyl-5-(piperidin-2-yl methoxy)benzamide (Compound 550)

To a solution of(S)-tert-butyl 2-((3-((1-(7-methoxyquinolin-5-yl)cyclopropyl)carbamoyl)-4-methylphenoxy)methyl)piperidine-1-carboxylate (200 mg, 330 μmol, 1.0 eq) in EtOAc (1.0 mL) was added HCl/EtOAc (4 M, 5.0 mL) slowly. The resulting mixture was stirred at 20° C. for 1 h. LCMS indicated that the starting material was consumed, and the desired mass was detected. The mixture was filtered, and the cake was washed with EtOAc (5.0 mL×3). The cake was dried under vacuum to give(S)—N-(1-(7-methoxyquinolin-5-yl)cyclopropyl)-2-methyl-5-(piperidin-2-yl methoxy)benzamide (120 mg, 269 μmol, 82% yield, HCl salt) as a yellow solid. M+H⁺=446.2 (LCMS); ¹H NMR (400 MHZ, DMSO-d₆) δ 9.65 (br d, J=8.1 Hz, 1H), 9.36 (s, 1H), 9.17 (br d, J=4.8 Hz, 1H), 9.12 (br s, 1H), 7.95 (dd, J=5.3, 8.3 Hz, 1H), 7.72 (d, J=2.3 Hz, 1H), 7.66 (d, J=1.9 Hz, 1H), 7.09 (d, J=8.4 Hz, 1H), 6.92 (dd, J=2.6, 8.3 Hz, 1H), 6.75 (d, J=2.5 Hz, 1H), 4.15-4.10 (m, 1H), 4.08-4.04 (m, 1H), 4.02 (s, 3H), 3.45-3.33 (m, 1H), 3.22 (br d, J=12.3 Hz, 1H), 2.88 (br d, J=6.8 Hz, 1H), 1.96 (s, 3H), 1.86-1.76 (m, 2H), 1.75-1.60 (m, 2H), 1.59-1.45 (m, 2H), 1.41 (br s, 2H), 1.31 (br s, 2H).

### Example 343: 5-((4-Methoxypyridin-2-yl)methoxy)-N-(1-(7-methoxyquinolin-5-yl)cyclopropyl)-2-methylbenzamide (Compound 561)

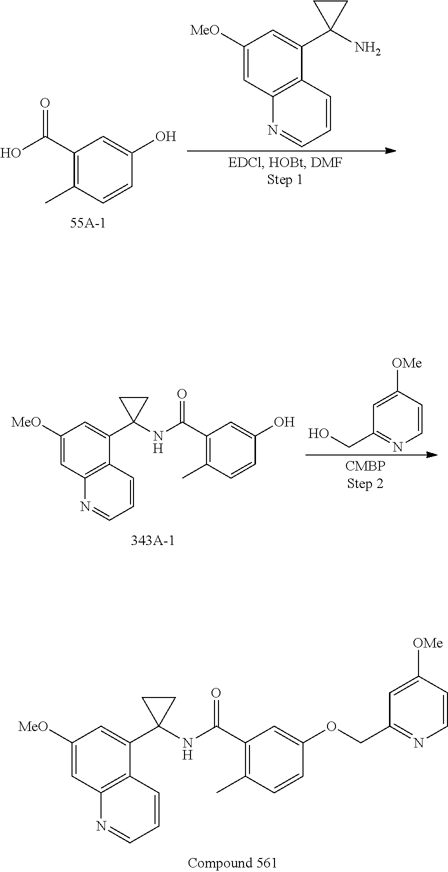

### Step 1: 5-Hydroxy-N-(1-(7-methoxyquinolin-5-yl)cyclopropyl)-2-methylbenzamide (343A-1)

To a solution of 5-hydroxy-2-methylbenzoic acid (700 mg, 4.60 mmol, 0.98 eq) and 1-(7-methoxyquinolin-5-yl)cyclopropanamine (1.00 g, 4.67 mmol, 1.0 eq) in DMF (15 mL) were added TEA (472 mg, 4.67 mmol, 650 μL, 1.0 eq), EDCI (939 mg, 4.90 mmol, 1.05 eq) and HOBt (126 mg, 933 μmol, 0.2 eq). The mixture was stirred at 20° C. for 3 h. LCMS indicated that the starting material was completely consumed. The mixture was poured into H₂O (40 mL) and extracted with EtOAc (20 mL×2). The combined organic layers were dried over Na₂SO₄, filtered, and concentrated under vacuum to give a residue which was purified by flash silica gel chromatography using a gradient of EtOAc/petroleum ether from 0/1 to 1/1. 5-Hydroxy-N-(1-(7-methoxyquinolin-5-yl)cyclopropyl)-2-methylbenzamide (1.10 g, 3.16 mmol, 68% yield) was obtained as a yellow solid. M+H⁺=349.0 (LCMS); ¹H NMR (400 MHZ, DMSO-d₆) δ 9.27 (s, 1H), 9.07 (s, 1H), 8.97-8.92 (m, 1H), 8.82 (dd, J=1.5, 4.3 Hz, 1H), 7.49-7.39 (m, 2H), 7.31 (d, J=2.5 Hz, 1H), 6.91 (d, J=8.4 Hz, 1H), 6.64 (dd, J=2.6, 8.3 Hz, 1H), 6.44 (d, J=2.6 Hz, 1H), 3.92 (s, 3H), 1.92 (s, 3H), 1.34-1.28 (m, 2H), 1.23-1.16 (m, 2H).

### Step 2: 5-((4-Methoxypyridin-2-yl)methoxy)-N-(1-(7-methoxyquinolin-5-yl)cyclopropyl)-2-methylbenzamide (Compound 561)

A mixture of 5-hydroxy-N-(1-(7-methoxyquinolin-5-yl)cyclopropyl)-2-methylbenzamide (70.0 mg, 201 μmol, 1.0 eq), (4-methoxypyridin-2-yl)methanol (55.9 mg, 402 μmol, 2.0 eq), and CMBP (72.7 mg, 302 μmol, 1.5 eq) in toluene (3.5 mL) was degassed and purged with N₂ three times. The mixture was stirred at 100° C. for 16 h under a N₂ atmosphere. LCMS indicated that the starting material was completely consumed. The reaction mixture was allowed to cool to room temperature, poured into H₂O (10 mL) and extracted with EtOAc (10 mL×4). The combined organic layers were dried over Na₂SO₄, filtered, and concentrated under vacuum to give a residue which was purified by preparative HPLC (Phenomenex Luna C18 column (80× 30 mm, 3 μm); flow rate: 25 mL/min; gradient: 1%-30% B over 8 min; mobile phase A: 0.04% aqueous HCl, mobile phase B: acetonitrile). 5-((4-Methoxypyridin-2-yl)methoxy)-N-(1-(7-methoxyquinolin-5-yl)cyclopropyl)-2-methylbenzamide (81.9 mg, 153 μmol, 76% yield, HCl salt) was obtained as a yellow solid. M+H⁺=470.2 (LCMS); ¹H NMR (400 MHZ, DMSO-d₆) δ=9.74-9.65 (m, 1H), 9.67 (br d, J=8.5 Hz, 1H), 9.40 (s, 1H), 9.19-9.12 (m, 1H), 8.70 (d, J=6.8 Hz, 1H), 7.95 (dd, J=5.3, 8.4 Hz, 1H), 7.77-7.64 (m, 2H), 7.56 (d, J=2.5 Hz, 1H), 7.46 (dd, J=2.6, 6.6 Hz, 1H), 7.11 (d, J=8.5 Hz, 1H), 7.00 (dd, J=2.6, 8.4 Hz, 1H), 6.87 (d, J=2.8 Hz, 1H), 5.36 (s, 2H), 4.03 (d, J=13.5 Hz, 6H), 1.98 (s, 3H), 1.45-1.37 (m, 2H), 1.35-1.27 (m, 2H).

### Example 344: N-(1-(7-Methoxyquinolin-5-yl)cyclopropyl)-2-methyl-5-(pyridin-4-yloxy)benzamide (Compound 584)

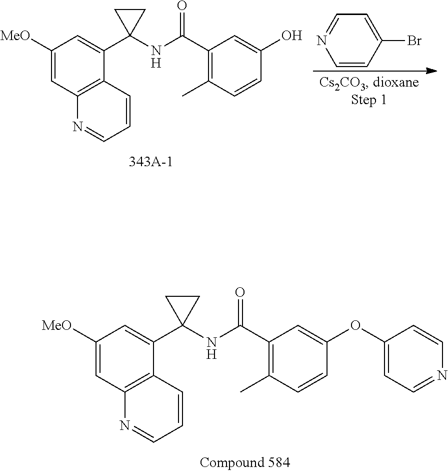

### Step 1: N-(1-(7-Methoxyquinolin-5-yl)cyclopropyl)-2-methyl-5-(pyridin-4-yloxy)benzamide (Compound 584)

To a solution of 5-hydroxy-N-(1-(7-methoxyquinolin-5-yl)cyclopropyl)-2-methylbenzamide (60.0 mg, 172 μmol, 1.0 eq) and 4-bromopyridine (81.6 mg, 517 μmol, 3.0 eq) in dioxane (4.0 mL) were added Cs₂CO₃ (112 mg, 344 μmol, 2.0 eq), Pd₂ (dba) 3 (15.8 mg, 17.2 μmol, 0.1 eq) and Xantphos (20.9 mg, 36.2 μmol, 0.2 eq). The mixture was degassed and purged with N₂ three times. The mixture was stirred at 110° C. for 16 h under a N₂ atmosphere. LCMS indicated that the starting material was completely consumed, and the desired mass was detected. The reaction mixture was allowed to cool to room temperature, poured into H₂O (4.0 mL) and extracted with DCM (4.0 mL×3). The combined organic layers were dried over Na₂SO₄, filtered, and concentrated under vacuum to give a residue which was purified by preparative HPLC (Waters Xbridge Prep OBD C18 column (150×40 mm, 10 μm); flow rate: 25 mL/min; gradient: 20%-50% B over 8 min; mobile phase A: 10 mM aqueous NH₄HCO₃, mobile phase B: acetonitrile). N-(1-(7-Methoxyquinolin-5-yl)cyclopropyl)-2-methyl-5-(pyridin-4-yloxy)benz amide (11.2 mg, 26.1 μmol, 15% yield) was obtained as a brown solid. M+H⁺=426.1 (LCMS); ¹H NMR (400 MHZ, DMSO-d₆) δ 9.18 (s, 1H), 8.91 (d, J=8.3 Hz, 1H), 8.79 (dd, J=1.3, 4.1 Hz, 1H), 8.43 (d, J=6.1 Hz, 2H), 7.46 (d, J=2.5 Hz, 1H), 7.38 (dd, J=4.3, 8.4 Hz, 1H), 7.30 (d, J=2.5 Hz, 1H), 7.25 (d, J=8.4 Hz, 1H), 7.09 (dd, J=2.5, 8.3 Hz, 1H), 6.89 (d, J=2.5 Hz, 1H), 6.86 (d, J=6.1 Hz, 2H), 3.91 (s, 3H), 2.04 (s, 3H), 1.40-1.27 (m, 2H), 1.23-1.13 (m, 2H).

### Example 345: (S)-4-Amino-N-(1-(7-methoxyquinolin-5-yl)cyclopropyl)-2-methyl-5-((1-methylazetidin-2-yl)methoxy)benzamide (Compound 597)

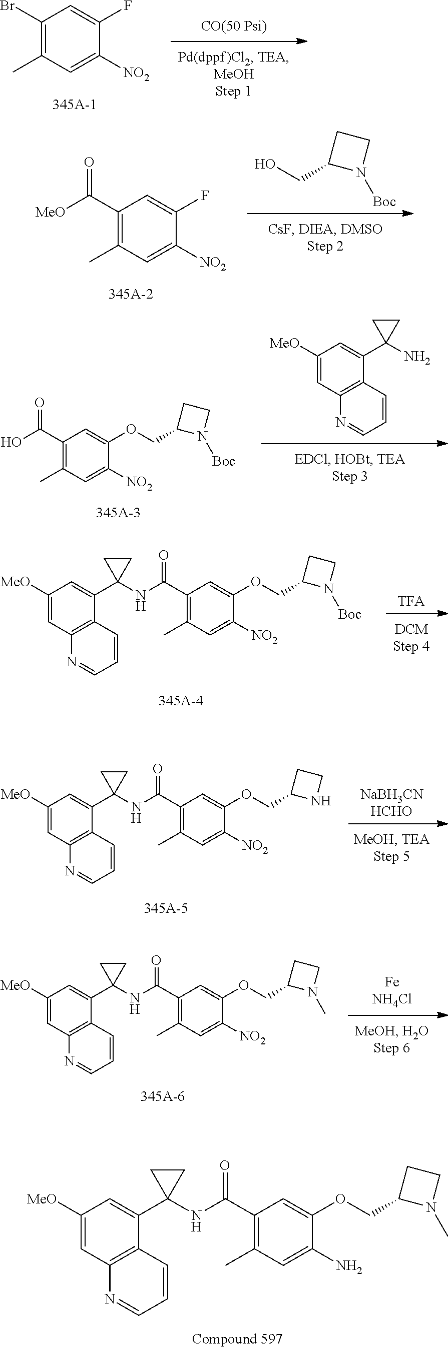

### Step 1: Methyl 5-fluoro-2-methyl-4-nitrobenzoate (345A-2)

To a solution of 1-bromo-5-fluoro-2-methyl-4-nitrobenzene (500 mg, 2.14 mmol, 1.0 eq) in MeOH (10 mL) were added TEA (1.73 g, 17.1 mmol, 2.38 mL, 8.0 eq) and Pd(dppf)Cl₂ (156 mg, 214 μmol, 0.1 eq). The mixture was degassed and purged with CO three times, then stirred at 80° C. for 16 h under a CO (50 psi) atmosphere. TLC indicated that the starting material was completely consumed. The reaction mixture was allowed to cool to room temperature and concentrated under vacuum to give a residue which was purified by preparative TLC (EtOAc/petroleum ether=1/5, Ry-0.5). Methyl 5-fluoro-2-methyl-4-nitrobenzoate (200 mg, 938 μmol, 44% yield) was obtained as a yellow solid. ¹H NMR (400 MHZ, CDCl₃) δ 7.93 (d, J=7.1 Hz, 1H), 7.83 (d, J=11.2 Hz, 1H), 3.96 (s, 3H), 2.64 (s, 3H).

### Step 2: (S)-5-((1-(tert-Butoxycarbonyl)azetidin-2-yl)methoxy)-2-methyl-4-nitrobenzoic acid (345A-3)

To a solution of methyl 5-fluoro-2-methyl-4-nitrobenzoate (180 mg, 844 μmol, 1.0 eq) and(S)-tert-butyl 2-(hydroxymethyl)azetidine-1-carboxylate (237 mg, 1.27 mmol, 1.5 eq) in DMSO (15 mL) were added CsF (257 mg, 1.69 mmol, 62.3 μL, 2.0 eq) and DIEA (327 mg, 2.53 mmol, 441 μL, 3.0 eq). The mixture was stirred at 120° C. for 16 h. LCMS indicated that the starting material was completely consumed, and the desired mass was detected. The reaction mixture was allowed to cool to room temperature. The reaction mixture was poured into H₂O (10 mL) and extracted with MTBE (6.0 mL×2). The aqueous layer was acidified to pH 5 with HCl (1 M aqueous). The mixture was extracted with EtOAc (6.0 mL×4). The combined organic layers were dried over Na₂SO₄, filtered, and concentrated under vacuum to give a residue which was purified by flash silica gel chromatography using a gradient of EtOAc/petroleum ether from 1/100 to 1/10. (S)-5-((1-(tert-Butoxycarbonyl)azetidin-2-yl)methoxy)-2-methyl-4-nitrobenzoic acid (200 mg, 546 μmol, 65% yield) was obtained as a yellow solid. M−56+H⁺=311.0 (LCMS).

### Step 3: (S)-tert-Butyl 2-((5-((1-(7-methoxyquinolin-5-yl)cyclopropyl)carbamoyl)-4-methyl-2-nitrophenoxy)methyl)azetidine-1-carboxylate (345A-4)

To a solution of(S)-5-((1-(tert-butoxycarbonyl)azetidin-2-yl)methoxy)-2-methyl-4-nitrobenzoic acid (198 mg, 539 μmol, 1.0 eq) and 1-(7-methoxyquinolin-5-yl)cyclopropanamine (57.8 mg, 270 μmol, 0.5 eq) in DMF (12 mL) were added TEA (54.6 mg, 539 μmol, 75.0 μL, 1.0 eq), EDCI (109 mg, 566 μmol, 1.05 eq) and HOBt (14.6 mg, 108 μmol, 0.2 eq). The mixture was stirred at 20° C. for 2 h. LCMS indicated that the starting material was completely consumed, and the desired mass was detected. The reaction mixture was poured into H₂O (15 mL) and extracted with EtOAc (6.0 mL×3). The combined organic layers were dried over Na₂SO₄, filtered, and concentrated under vacuum to give a residue which was purified by flash silica gel chromatography using a gradient of EtOAc/petroleum ether from 1/100 to 4/5. (S)-tert-Butyl 2-((5-((1-(7-methoxyquinolin-5-yl)cyclopropyl)carbamoyl)-4-methyl-2-nitro phenoxy)methyl)azetidine-1-carboxylate (90.0 mg, 160 μmol, 30% yield) was obtained as a yellow solid. M+H⁺=563.3 (LCMS).

### Step 4: (S)-5-(Azetidin-2-ylmethoxy)-N-(1-(7-methoxyquinolin-5-yl)cyclopropyl)-2-methyl-4-nitrobenzamide (345A-5)

To a solution of(S)-tert-butyl 2-((5-((1-(7-methoxyquinolin-5-yl)cyclopropyl)carbamoyl)-4-methyl-2-nitrophenoxy)methyl)azetidine-1-carboxylate (90.0 mg, 160 μmol, 1.0 eq) in DCM (2.0 mL) was added TFA (2.31 g, 20.3 mmol, 1.5 mL, 127 eq). The mixture was stirred at 20° C. for 1 h. LCMS indicated that the starting material was completely consumed, and the desired mass was detected. The reaction mixture was concentrated under vacuum to the crude (S)-5-(azetidin-2-ylmethoxy)-N-(1-(7-methoxyquinolin-5-yl)cyclopropyl)-2-methyl-4-nitrobenzamide (80.0 mg, 139 μmol, 87% yield, TFA salt) as a yellow oil. M+H⁺=463.1 (LCMS).

### Step 5: (S)—N-(1-(7-Methoxyquinolin-5-yl)cyclopropyl)-2-methyl-5-((1-methylazetidin-2-yl)methoxy)-4-nitrobenzamide (345A-6)

To a solution of(S)-5-(azetidin-2-ylmethoxy)-N-(1-(7-methoxyquinolin-5-yl)cyclopropyl)-2-methyl-4-nitrobenzamide (80.0 mg, 139 μmol, 1.0 eq, TFA salt) in MeOH (2.0 mL) was added TEA (30.0 μL), followed by formaldehyde (22.5 mg, 278 μmol, 7.65 μL, 37% purity in water, 2.0 eq). The resulting mixture was adjusted to pH 6 with a small amount of AcOH. The mixture was stirred at 20° C. for 30 min, then NaBH₃CN (17.4 mg, 278 μmol, 2.0 eq) was added. The resulting mixture was stirred at 20° C. for another 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give the crude(S)—N-(1-(7-methoxyquinolin-5-yl)cyclopropyl)-2-methyl-5-((1-methylazetidin-2-yl)methoxy)-4-nitrobenzamide (65.0 mg) as a yellow oil. M+H⁺=477.2 (LCMS).

### Step 6: (S)-4-Amino-N-(1-(7-methoxyquinolin-5-yl)cyclopropyl)-2-methyl-5-((1-methylazetidin-2-yl)methoxy)benzamide (Compound 597)

To a solution of(S)—N-(1-(7-methoxyquinolin-5-yl)cyclopropyl)-2-methyl-5-((1-methylazetidin-2-yl)methoxy)-4-nitrobenzamide (60.0 mg, 126 μmol, 1.0 eq) in MeOH (6.0 mL) and H₂O (1.2 mL) were added NH₄Cl (33.7 mg, 630 μmol, 5.0 eq) and iron powder (35.2 mg, 630 μmol, 5.0 eq). The mixture was stirred at 80° C. for 2 h. LCMS indicated that the starting material was completely consumed, and the desired mass was detected. The reaction mixture was allowed to cool to room temperature, poured into H₂O (7.0 mL) and extracted with EtOAc (5.0 mL×3). The combined organic layers were dried over Na₂SO₄, filtered, and concentrated under vacuum to give a residue which was purified by preparative HPLC (Waters Xbridge Prep OBD C18 (150×40 mm, 10 μm); flow rate: 50 mL/min; gradient: 10%-40% B over 8 min; mobile phase A: 10 mM aqueous NH₄HCO₃, mobile phase B: acetonitrile). (S)-4-Amino-N-(1-(7-methoxyquinolin-5-yl)cyclopropyl)-2-methyl-5-((1-methylazetidin-2-yl)methoxy)benzamide (6.50 mg, 14.6 μmol, 12% yield) was obtained as a brown solid. M+H⁺=447.3 (LCMS); ¹H NMR (400 MHZ, DMSO-d₆) δ 9.01 (d, J=8.3 Hz, 1H), 8.81 (d, J=4.0 Hz, 1H), 8.70 (s, 1H), 7.53-7.36 (m, 2H), 7.29 (d, J=2.3 Hz, 1H), 6.61 (s, 1H), 6.35 (s, 1H), 4.98-4.83 (m, 2H), 3.91 (s, 3H), 3.87-3.74 (m, 2H), 3.25 (br d, J=8.5 Hz, 2H), 2.78-2.69 (m, 1H), 2.22 (s, 3H), 2.03-1.84 (m, 5H), 1.31 (br s, 2H), 1.20-1.11 (m, 2H).

### Example 346: (S)—N-(1-(7-Isopropoxyquinolin-5-yl)cyclopropyl)-2-methyl-5-((1-methylazetidin-2-yl)methoxy)benzamide (Compound 587)

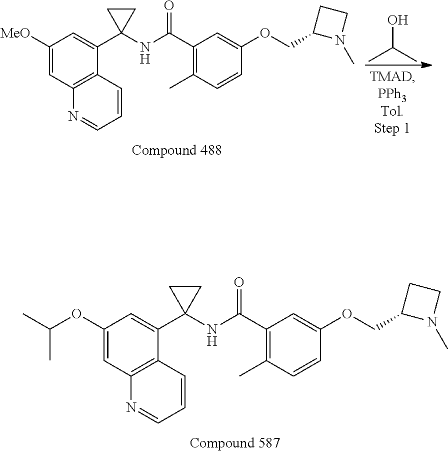

### Step 1: (S)—N-(1-(7-Isopropoxyquinolin-5-yl)cyclopropyl)-2-methyl-5-((1-methylazetidin-2-yl)methoxy)benzamide (Compound 587)

To a solution of(S)—N-(1-(7-hydroxyquinolin-5-yl)cyclopropyl)-2-methyl-5-((1-methylazetidin-2-yl)methoxy)benzamide (18.0 mg, 43.1 μmol, 1.0 eq) and propan-2-ol (7.77 mg, 129 μmol, 9.90 μL, 3.0 eq) in toluene (2.0 mL) were added TMAD (22.2 mg, 129 μmol, 3.0 eq) and PPh₃ (33.9 mg, 129 μmol, 3.0 eq) at 25° C. The mixture was stirred at 110° C. for 16 h under a N₂ atmosphere. LCMS indicated that the starting material was completely consumed, and the desired mass was detected. The reaction mixture was allowed to cool to room temperature and then concentrated under vacuum to give a residue which was purified by preparative HPLC (Phenomenex Gemini C18 column (80×30 mm, 3 μm); flow rate: 60 mL/min; gradient: 1%-25% B over 8 min; mobile phase A: 0.04% aqueous HCl, mobile phase B: acetonitrile). (S)—N-(1-(7-Isopropoxyquinolin-5-yl)cyclopropyl)-2-methyl-5-((1-methylazetidin-2-yl)methoxy)benzamide (3.60 mg, 6.99 μmol, 16% yield) was obtained as a pale yellow gum. M+H⁺=460.1 (LCMS); ¹H NMR (400 MHZ, DMSO-d₆) δ 10.51-10.34 (m, 1H), 9.49-9.34 (m, 1H), 9.24 (s, 1H), 9.06 (br d, J=2.8 Hz, 1H), 7.85-7.71 (m, 1H), 7.61 (br s, 1H), 7.52-7.46 (m, 1H), 7.10 (d, J=8.6 Hz, 1H), 6.93 (br d, J=2.5 Hz, 1H), 6.75 (d, J=2.4 Hz, 1H), 4.86 (s, 1H), 4.67-4.58 (m, 1H), 4.35-4.29 (m, 1H), 4.24 (br d, J=2.9 Hz, 1H), 4.04-3.98 (m, 1H), 3.89-3.82 (m, 1H), 2.84-2.81 (m, 3H), 2.33 (br d, J=1.8 Hz, 2H), 1.96 (s, 3H), 1.41 (d, J=6.0 Hz, 8H), 1.28 (br s, 2H).

### Example 347: (S)—N-(1-(7-(Benzyloxy)quinolin-5-yl)cyclopropyl)-2-methyl-5-((1-methyl

azetidin-2-yl)methoxy)benzamide (Compound 598)

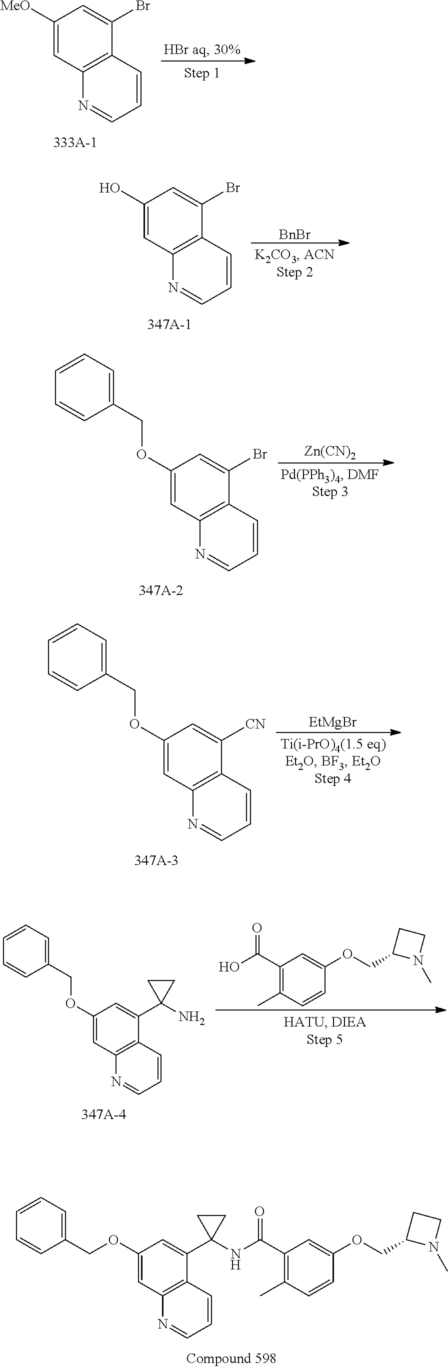

### Step 1: 5-Bromoquinolin-7-ol (347A-1)

To a solution of 5-bromo-7-methoxy-quinoline (2.00 g, 8.40 mmol, 1.0 eq) in HBr (30.0 g, 110 mmol, 20 mL, 30% purity in H₂O, 13 eq). The mixture was stirred at 130° C. for 24 h. LCMS indicated that the starting material was completely consumed, and the desired mass was detected. The reaction mixture was allowed to cool to 0° C. and slowly adjusted to pH 6 with saturated aqueous NaHCO₃. The mixture was extracted with DCM (50 mL×5). The combined organic layers were dried over Na₂SO₄, filtered, and concentrated under vacuum to give a crude product, which was used in the next step without any further purification. 5-Bromoquinolin-7-ol (3.50 g) was obtained as a yellow solid. M+H⁺=223.9 (LCMS); ¹H NMR (400 MHZ, DMSO-d₆) δ 8.78 (dd, J=1.4, 4.2 Hz, 1H), 8.31 (d, J=8.4 Hz, 1H), 7.52 (d, J=2.3 Hz, 1H), 7.41 (dd, J=4.2, 8.4 Hz, 1H), 7.26 (d, J=1.9 Hz, 1H).

### Step 2: 7-(Benzyloxy)-5-bromoquinoline (347A-2)

To a solution of 5-bromoquinolin-7-ol (1.00 g, 2.68 mmol, 1.0 eq) in DMF (20 mL) was added (bromomethyl)benzene (504 mg, 295 mmol, 350 μL 1.1 eq), followed by K₂CO₃ (1.11 g, 8.03 mmol, 3.0 eq). The resulting mixture was stirred at 80° C. for 16 h. LCMS indicated that the starting material was completely consumed, and the desired mass was detected. The reaction mixture was allowed to cool to room temperature, poured into H₂O (50 mL) and extracted with EtOAc (20 mL×4). The combined organic layers were dried over Na₂SO₄, filtered, and concentrated under vacuum to give a residue which was purified by flash silica gel chromatography using a gradient of EtOAc/petroleum ether from 0/1 to 1/5. 7-(Benzyloxy)-5-bromoquinoline (800 mg, 2.54 mmol, 95% yield) was obtained as yellow oil M+H⁺=313.9 (LCMS).

### Step 3: 7-(Benzyloxy)quinoline-5-carbonitrile (347A-3)

To a solution of 7-(benzyloxy)-5-bromoquinoline (500 mg, 1.59 mmol, 1.0 eq) in DMF (10 mL) were added Zn(CN)₂ (374 mg, 3.18 mmol, 2.0 eq), and Pd(PPh₃)₄ (184 mg, 159 μmol, 0.1 eq). The resulting mixture was degassed and purged with N₂ three times, and then the mixture was stirred at 120° C. for 16 h under a N₂ atmosphere. TLC indicated that the starting material was completely consumed. The reaction mixture was allowed to cool to room temperature, poured into H₂O (30 mL) and extracted with EtOAc (20 mL×5). The combined organic layers were dried over Na₂SO₄, filtered, and concentrated under vacuum to give a residue which was purified by flash silica gel chromatography using a gradient of EtOAc/petroleum ether from 0/1 to 1/1. 7-(Benzyloxy)quinoline-5-carbonitrile (400 mg, 1.54 mmol, 97% yield) was obtained as a white solid. ¹H NMR (400 MHZ, DMSO-d₆) δ 8.99 (dd, J=1.5, 4.3 Hz, 1H), 8.41 (d, J=8.1 Hz, 1H), 8.10 (d, J=2.5 Hz, 1H), 7.87 (d, J=2.3 Hz, 1H), 7.63 (dd, J=4.3, 8.4 Hz, 1H), 7.53 (d, J=7.3 Hz, 2H), 7.47-7.34 (m, 3H), 5.37 (s, 2H).

### Step 4: 1-(7-(Benzyloxy)quinolin-5-yl)cyclopropanamine (347A-4)

A mixture of 7-(benzyloxy)quinoline-5-carbonitrile (200 mg, 771 μmol, 1.0 eq) in anhydrous Et₂O (20 mL) was degassed and purged with N₂ three times. The mixture was stirred at −78° C. To this mixture was added Ti(i-PrO)₄ (329 mg, 1.16 mmol, 341 μL, 1.5 eq) slowly, and then EtMgBr (3 M in Et₂O, 566 μL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. BF₃·Et₂O (219 mg, 1.54 mmol, 190 μL, 2.0 eq) was added slowly with no obvious temperature change. The mixture was stirred at 20° C. for another 1 h. TLC indicated that the starting material was completely consumed. The reaction mixture was added into a mixture of HCl (1 M aqueous) (10 mL) and MTBE (5.0 mL) and extracted with MTBE (5.0 mL×2). The aqueous layer was basified to pH 8 by using NaOH (2 M aqueous), and a precipitate was formed. The mixture was filtered through a pad of Celite and the slurry was washed with DCM several times. The combined filtrate was extracted with DCM (10 mL×3).

The combined organic layers were dried over Na₂SO₄, filtered, and concentrated under vacuum to give a residue which was purified by preparative TLC (MeOH/DCM=1/10, R_{f}=0.2). 1-(7-(Benzyloxy)quinolin-5-yl)cyclopropanamine (30.0 mg, 103 μmol, 13% yield) was obtained as a yellow oil. M+H⁺=291.1 (LCMS).

### Step 5: (S)—N-(1-(7-(Benzyloxy)quinolin-5-yl)cyclopropyl)-2-methyl-5-((1-methyl azetidin-2-yl)methoxy)benzamide (Compound 598)

To a solution of(S)-2-methyl-5-((1-methylazetidin-2-yl)methoxy)benzoic acid (30.0 mg, 128 μmol, 1.0 eq) and 1-(7-(benzyloxy)quinolin-5-yl)cyclopropanamine (39.0 mg, 134 μmol, 1.1 eq) in DMF (2.0 mL) were added HATU (145 mg, 383 μmol, 3.0 eq) and DIEA (33.0 mg, 255 μmol, 44.0 μL, 2.0 eq). The mixture was stirred at 20° C. for 16 h. LCMS indicated that the starting material was completely consumed, and the desired mass was detected. The mixture was poured into H₂O (10 mL) and extracted with EtOAc (5.0 mL×5). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 1%-35% B over 8 min; mobile phase A: 0.04% aqueous HCl, mobile phase B: acetonitrile). (S)—N-(1-(7-(Benzyloxy)quinolin-5-yl)cyclopropyl)-2-methyl-5-((1-methyl azetidin-2-yl)methoxy)benzamide (12.7 mg, 22.6 μmol, 18% yield, HCl salt) was obtained as a yellow solid. M+H⁺=508.1 (LCMS); ¹H NMR (400 MHZ, DMSO-d₆) δ 10.86-10.61 (m, 1H), 9.62-9.45 (m, 1H), 9.31 (s, 1H), 9.11 (br d, J=4.8 Hz, 1H), 7.96-7.82 (m, 1H), 7.75 (s, 1H), 7.67 (br s, 1H), 7.57 (br d, J=7.3 Hz, 2H), 7.49-7.35 (m, 3H), 7.10 (d, J=8.4 Hz, 1H), 6.92 (dd, J=2.6, 8.4 Hz, 1H), 6.76 (d, J=2.5 Hz, 1H), 5.36 (s, 2H), 4.70-4.54 (m, 1H), 4.41-4.35 (m, 1H), 4.22 (br dd, J=2.6, 11.2 Hz, 1H), 3.99 (br dd, J=4.8, 9.0 Hz, 1H), 3.89-3.82 (m, 1H), 2.82-2.66 (m, 3H), 2.33 (br s, 2H), 1.96 (s, 3H), 1.40 (br s, 2H), 1.30 (br s, 2H).

### Example 348: (S)—N-(1-(7-Bromoquinolin-5-yl)cyclopropyl)-2-methyl-5-((1-methylazetidin-2-yl)methoxy)benzamide (Compound 563)

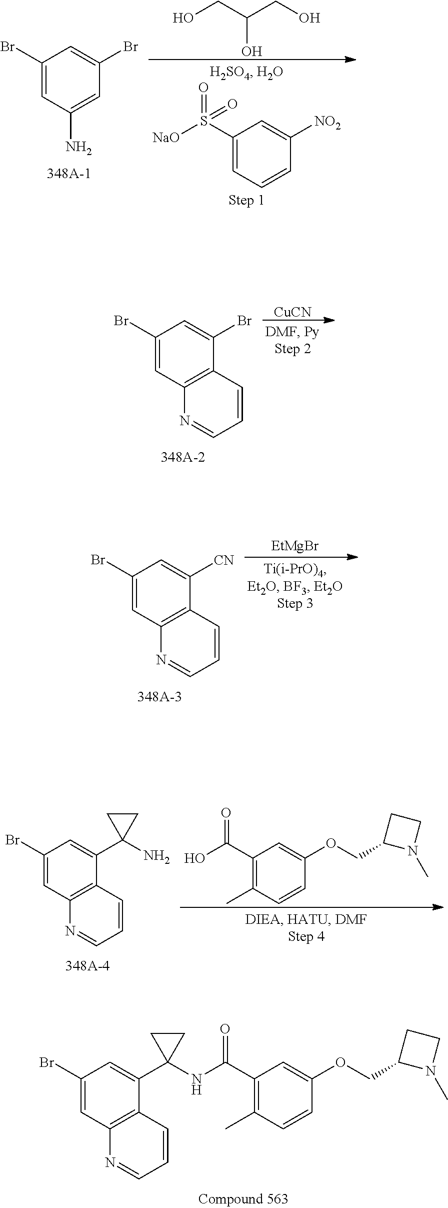

### Step 1: 5,7-Dibromoquinoline (348A-2)

To a solution of 3,5-dibromoaniline (10.0 g, 39.8 mmol, 1.0 eq) and sodium 3-nitrobenzenesulfonate (26.9 g, 119 mmol, 3.0 eq) in a mixture of H₂SO₄ (56 mL) and H₂O (72 mL) was added propane-1,2,3-triol (14.7 g, 159 mmol, 11.9 mL, 4.0 eq) at 100° C. The mixture was degassed and purged with N₂ three times. The mixture was stirred at 130° C. for 4 h under a N₂ atmosphere. LCMS indicated that the starting material was completely consumed, and the desired mass was detected. The reaction mixture was allowed to cool to room temperature, poured into ice water (200 mL). The aqueous layer was basified to pH 9-10 by using saturated aqueous NH₃H₂O and extracted with EtOAc (50 mL×3). The combined organic layers were dried over Na₂SO₄, filtered, and concentrated under vacuum to give a residue which was purified by flash silica gel chromatography using a gradient of EtOAc/petroleum ether from 0/1 to 1/10. 5,7-Dibromoquinoline (25.0 g, 87.1 mmol, 73% yield) was obtained as a white solid. M+H⁺=285.8 (LCMS); ¹H NMR (400 MHz, DMSO-d₆) δ 9.05-8.98 (m, 1H), 8.52 (d, J=8.3 Hz, 1H), 8.32 (s, 1H), 8.22 (d, J=1.7 Hz, 1H), 7.75 (dd, J=4.2, 8.5 Hz, 1H).

### Step 2: 7-Bromoquinoline-5-carbonitrile (348A-3)

To a solution of 5,7-dibromoquinoline (1.00 g, 3.48 mmol, 1.0 eq) in DMF (5.0 mL) were added Py (6.34 g, 80.2 mmol, 6.47 mL, 23 eq) and CuCN (312 mg, 3.48 mmol, 761 μL, 1.0 eq) at 20° C. The mixture was degassed and purged with N₂ three times. The mixture was stirred at 150° C. for 16 h under a N₂ atmosphere. LCMS indicated that the starting material was completely consumed, and the desired mass was detected. The reaction mixture was allowed to cool to room temperature, poured into H₂O (5.0 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/10. 7-Bromoquinoline-5-carbonitrile (1.00 g, crude) was obtained as a white solid. M+H⁺=233.1 (LCMS).

### Step 3: 1-(7-Bromoquinolin-5-yl)cyclopropanamine (348A-4)

A mixture of 7-bromoquinoline-5-carbonitrile (500 mg, 2.15 mmol, 1.0 eq) in anhydrous Et₂O (50 mL) was degassed and purged with N₂ three times. The mixture was stirred with a mechanical stirrer at −78° C. To this mixture was added Ti(i-PrO)₄ (915 mg, 3.22 mmol, 950 μL, 1.5 eq) slowly, and then EtMgBr (3 M in Et₂O, 1.57 mL, 2.2 eq) was added dropwise to maintain the temperature between −78° C. and −75° C. over 5 min under a N₂ atmosphere. After the addition was complete, the resulting mixture was stirred at the same temperature for 10 min and warmed to 20° C. over 1 h. BF₃·Et₂O (609 mg, 4.29 mmol, 529 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (50 mL) and MTBE (25 mL) and extracted with MTBE (25 mL×2). The aqueous layer was basified to pH 8 by using NaOH (2 M aqueous), and a precipitate was formed. The mixture was filtered through a pad of Celite and the slurry was washed with DCM several times. The combined filtrate was extracted with DCM (20 mL×4). The combined organic layers were dried over Na₂SO₄, filtered, and concentrated under vacuum to give a residue which was purified by preparative HPLC (Phenomenex Luna C18 column (80× 40 mm, 3 μm); flow rate: 25 mL/min; gradient: 1%-30% B over 7 min; mobile phase A: 0.04% aqueous HCl, mobile phase B: acetonitrile). 1-(7-Bromoquinolin-5-yl)cyclopropanamine (20.0 mg, 76.0 μmol, 4% yield, HCl salt) was obtained as a white solid. M+H⁺=263.0 (LCMS); ¹H NMR (400 MHZ, CD₃OD) δ 9.55 (d, J=8.6 Hz, 1H), 9.31 (d, J=5.0 Hz, 1H), 8.55 (s, 1H), 8.37 (d, J=1.3 Hz, 1H), 8.20 (dd, J=5.2, 8.7 Hz, 1H), 1.93-1.66 (m, 2H), 1.60-1.35 (m, 2H).

### Step 4: (S)—N-(1-(7-Bromoquinolin-5-yl)cyclopropyl)-2-methyl-5-((1-methylazetidin-2-yl)methoxy)benzamide (Compound 563)

To a solution of 1-(7-bromoquinolin-5-yl)cyclopropanamine (20.0 mg, 76.1 μmol, 1.0 eq) and (S)-2-methyl-5-((1-methylazetidin-2-yl)methoxy)benzoic acid (17.9 mg, 76.1 μmol, 1.0 eq) in DMF (3.0 mL) were added DIEA (49.1 mg, 380 μmol, 66.2 μL, 5.0 eq) and HATU (57.8 mg, 152 μmol, 2.0 eq). The mixture was stirred at 20° C. for 2 h. LCMS indicated that the starting material was completely consumed, and the desired mass was detected. The mixture was treated with H₂O (3.0 mL) and extracted with EtOAc (3.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 1%-35% B over 8 min; mobile phase A: 0.04% aqueous HCl, mobile phase B: acetonitrile). (S)—N-(1-(7-Bromoquinolin-5-yl)cyclopropyl)-2-methyl-5-((1-methylazetidin-2-yl)methoxy)benzamide (18.0 mg, 34.1 μmol, 45% yield, HCl salt) was obtained as a white solid. M+H⁺=480.0 (LCMS); ¹H NMR (400 MHZ, DMSO-d₆) δ 10.31-10.08 (m, 1H), 9.22 (s, 1H), 9.09 (d, J=8.3 Hz, 1H), 9.00-8.92 (m, 1H), 8.19 (d, J=1.8 Hz, 1H), 7.97 (d, J=1.9 Hz, 1H), 7.73-7.62 (m, 1H), 7.15-7.04 (m, 1H), 6.92 (dd, J=2.7, 8.2 Hz, 1H), 6.72 (d, J=2.8 Hz, 1H), 4.71-4.49 (m, 1H), 4.36-4.17 (m, 2H), 4.00 (br dd, J=4.9, 9.9 Hz, 1H), 3.85 (br d, J=3.4 Hz, 1H), 2.82 (d, J=5.0 Hz, 3H), 2.37-2.30 (m, 2H), 1.95 (s, 3H), 1.37 (br s, 2H), 1.28 (br s, 2H).

### Example 349: 5-(2-Aminopropoxy)-2-methyl-N-(1-(7-methylquinolin-5-yl)cyclopropyl)benzamide (Compound 497)

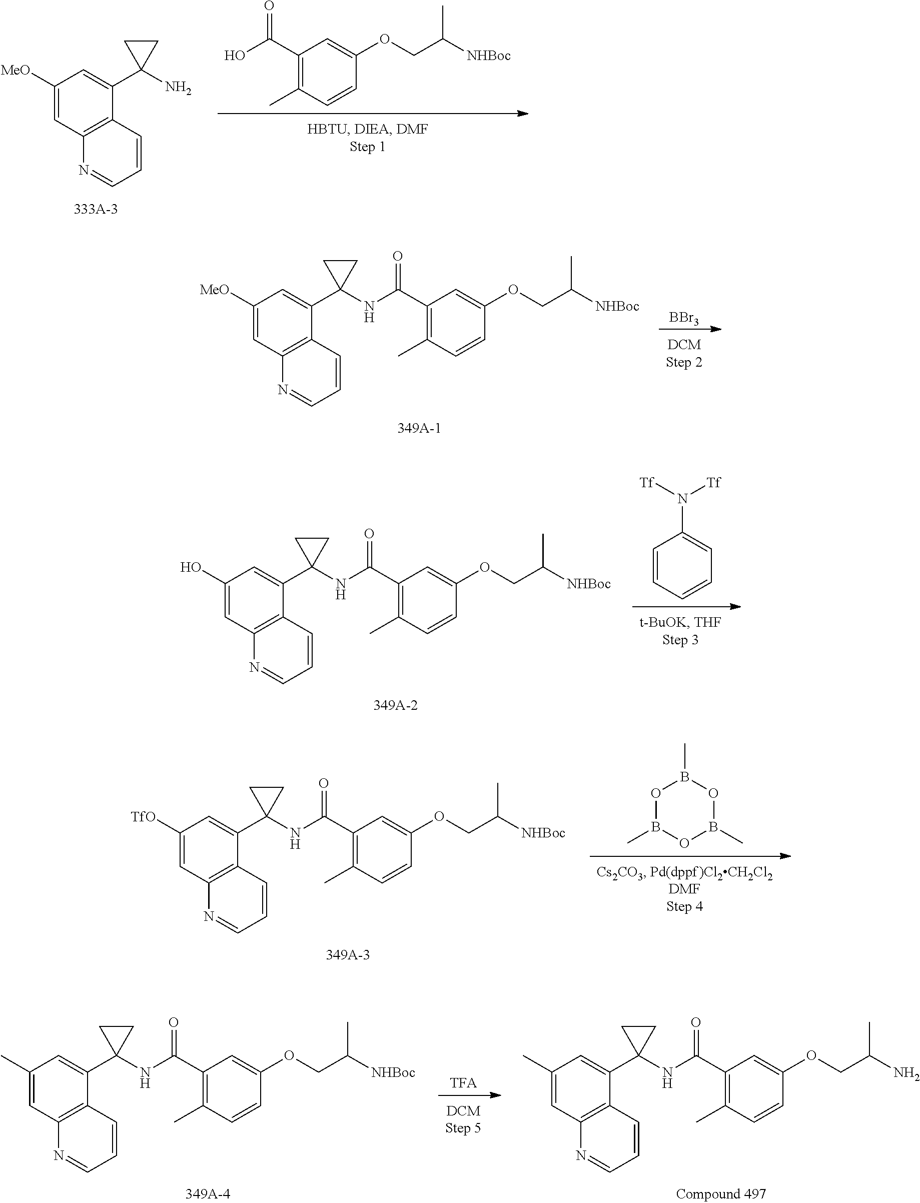

### Step 1: tert-Butyl(1-(3-((1-(7-methoxyquinolin-5-yl)cyclopropyl)carbamoyl)-4-methylphenoxy)propan-2-yl)carbamate (349A-1)

To a solution of 1-(7-methoxyquinolin-5-yl)cyclopropanamine (300 mg, 1.40 mmol, 1.0 eq) and 5-(2-((tert-butoxycarbonyl)amino)propoxy)-2-methylbenzoic acid (390 mg, 1.26 mmol, 0.9 eq) in DMF (5.0 mL) were added HBTU (1.33 g, 3.50 mmol, 2.5 eq) and DIEA (724 mg, 5.60 mmol, 976 μL, 4.0 eq). The mixture was stirred at 20° C. for 16 h. LCMS indicated that the starting material was completely consumed. The mixture was concentrated under vacuum to give a residue which was purified by flash silica gel chromatography using a gradient of EtOAc/petroleum ether from 0/1 to 1/0. tert-Butyl(1-(3-((1-(7-methoxyquinolin-5-yl)cyclopropyl)carbamoyl)-4-methylphenoxy)propan-2-yl)carbamate (485 mg, 959 μmol, 68% yield) was obtained as a yellow oil. M+H⁺=506.3 (LCMS).

### Step 2: tert-Butyl(1-(3-((1-(7-hydroxyquinolin-5-yl)cyclopropyl)carbamoyl)-4-methylphenoxy)propan-2-yl)carbamate (349A-2)

To a solution of tert-butyl(1-(3-((1-(7-methoxyquinolin-5-yl)cyclopropyl)carbamoyl)-4-methylphenoxy)propan-2-yl)carbamate (485 mg, 959 μmol, 1.0 eq) in DCM (10 mL) was added BBr₃ (3.60 g, 14.4 mmol, 1.39 mL, 15 eq) at −78° C. The mixture was stirred at −78° C. under a N₂ atmosphere for 1 h. The mixture was stirred at 20° C. for 16 h. LCMS indicated that the starting material was completely consumed. The mixture was poured into saturated aqueous NaHCO₃ (30 mL), then the mixture was concentrated under vacuum to give the crude product tert-butyl(1-(3-((1-(7-hydroxyquinolin-5-yl)cyclopropyl)carbamoyl)-4-methylphenoxy)propan-2-yl)carbamate (108 mg) as a yellow oil. M+H⁺=492.2 (LCMS).

### Step 3: 5-(1-(5-(2-((tert-Butoxycarbonyl)amino)propoxy)-2-methylbenzamido)cyclopropyl)quinolin-7-yl trifluoromethanesulfonate (349A-3)

To a solution of tert-butyl(1-(3-((1-(7-hydroxyquinolin-5-yl)cyclopropyl)carbamoyl)-4-methylphenoxy)propan-2-yl)carbamate (108 mg, 220 μmol, 1.0 eq) in THF (2.0 mL) was added 1-BuOK (36.9 mg, 330 μmol, 1.5 eq) at 0° C. The mixture was stirred at 0° C. for 15 min. Then 1,1,1-trifluoro-N-phenyl-N-((trifluoromethyl)sulfonyl)methanesulfonamide (117 mg, 330 μmol, 1.5 eq) was added at 0° C. The mixture was stirred at 0° C. for 30 min. LCMS indicated that the starting material was completely consumed. The mixture was quenched by saturated aqueous NaHCO₃ (5.0 mL), and extracted with DCM (5.0 mL×2). The combined organic layers were dried over Na₂SO₄, filtered, and concentrated under vacuum to give a residue which was purified by preparative TLC (EtOAc/petroleum ether=1/1, R_{f}=0.46). 5-(1-(5-(2-((tert-Butoxycarbonyl)amino)propoxy)-2-methylbenzamido)cyclopropyl)quinolin-7-yl trifluoromethanesulfonate (75.0 mg, 120 μmol, 54% yield) was obtained as a colourless oil. M+H⁺=624.2 (LCMS).

### Step 4: tert-Butyl(1-(4-methyl-3-((1-(7-methylquinolin-5-yl)cyclopropyl)carbamoyl)phenoxy)propan-2-yl)carbamate (349A-4)

To a solution of 5-(1-(5-(2-((tert-butoxycarbonyl)amino)propoxy)-2-methylbenzamido)cyclopropyl)quinolin-7-yl trifluoromethanesulfonate (75.0 mg, 120 μmol, 1.0 eq) and 2,4,6-trimethyl-1,3,5,2,4,6-trioxatriborinane (60.4 mg, 241 μmol, 67.3 μL, 50% purity, 2.0 eq) in DMF (1.5 mL) were added Cs₂CO₃ (129 mg, 397 μmol, 3.3 eq) and Pd(dppf)Cl₂·CH₂Cl₂ (9.82 mg, 12.0 μmol, 0.1 eq). The mixture was stirred at 110° C. under a N₂ atmosphere for 2 h. LCMS indicated that the starting material was completely consumed. The reaction mixture was allowed to cool to room temperature, poured into H₂O (5.0 mL) and extracted with EtOAc (5.0 mL×3). The combined organic layers were dried over Na₂SO₄, filtered, and concentrated under vacuum to give the crude product tert-butyl(1-(4-methyl-3-((1-(7-methylquinolin-5-yl)cyclopropyl)carbamoyl)phenoxy)propan-2-yl)carbamate (80.0 mg) as a brown liquid. M+H⁺=490.3 (LCMS).

### Step 5: 5-(2-Aminopropoxy)-2-methyl-N-(1-(7-methylquinolin-5-yl)cyclopropyl)benzamide (Compound 497)

To a solution of tert-butyl(1-(4-methyl-3-((1-(7-methylquinolin-5-yl)cyclopropyl) carbamoyl)phenoxy)propan-2-yl)carbamate (80.0 mg, 163 μmol, 1.0 eq) in DCM (1.0 mL) was added TFA (1.0 mL). The mixture was stirred at 20° C. for 2 h. LCMS indicated that the starting material was completely consumed. The mixture was concentrated under vacuum to give a residue which was purified by preparative HPLC (Phenomenex Luna C18 column (75× 30 mm, 3 μm); flow rate: 25 mL/min; gradient: 1%-25% B over 8 min; mobile phase A: 0.1% aqueous TFA, mobile phase B: acetonitrile). 5-(2-Aminopropoxy)-2-methyl-N-(1-(7-methylquinolin-5-yl)cyclopropyl)benzamide (36.2 mg, 71.9 μmol, 44% yield, TFA salt) as a white solid. M+H⁺=390.2 (LCMS); ¹H NMR (400 MHZ, DMSO-d₆) δ 9.31 (br d, J=7.88 Hz, 1H), 9.18 (s, 1H), 9.03 (br d, J=3.50 Hz, 1H), 7.98 (br s, 3H), 7.84 (br d, J=6.13 Hz, 2H), 7.76 (br dd, J=8.25, 4.13 Hz, 1H), 7.09 (d, J=8.50 Hz, 1H), 6.90 (dd, J=8.32, 2.56 Hz, 1H), 6.68 (d, J=2.50 Hz, 1H), 4.03 (dd, J=10.26, 3.75 Hz, 1H), 3.86 (dd, J=10.19, 7.07 Hz, 1H), 3.62-3.47 (m, 1H), 2.58 (s, 3H), 1.96 (s, 3H), 1.37 (br s, 2H), 1.30-1.16 (m, 5H).

### Example 350: (S)-2-Methyl-5-((1-methylazetidin-2-yl)methoxy)-N-(1-(7-methylquinolin-5-yl)cyclopropyl)benzamide (Compound 581)

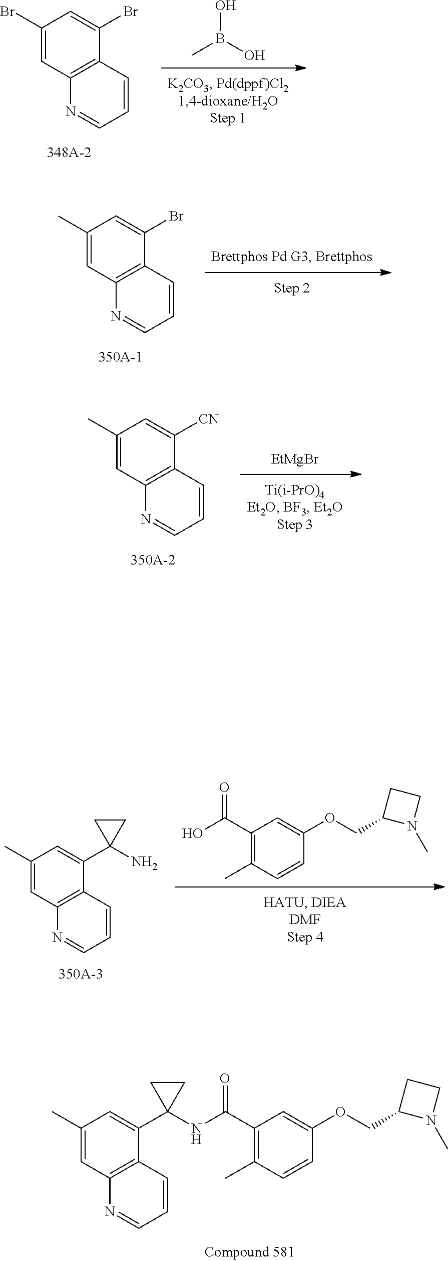

### Step 1: 5-Bromo-7-methylquinoline (350A-1)

To a solution of 5,7-dibromoquinoline (1.50 g, 5.23 mmol, 1.0 eq) and methylboronic acid (313 mg, 5.23 mmol, 1.0 eq) in a mixture of 1,4-dioxane (45 mL) and H₂O (11 mL) were added Pd(dppf)Cl₂ (382 mg, 523 μmol, 0.1 eq) and K₂CO₃ (1.44 g, 10.5 mmol, 2.0 eq). The mixture was degassed and purged with N₂ three times, and then stirred at 70° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (20 mL×2). The combined organic layers were dried over Na₂SO₄ filtered, and concentrated under vacuum to give a residue which was purified by flash silica gel chromatography using a gradient of EtOAc/petroleum ether from 0/1 to 1/2. 5-Bromo-7-methylquinoline (1.00 g, 4.50 mmol, 43% yield) was obtained as a white solid. M+H⁺=222.1 (LCMS).

### Step 2: 7-Methylquinoline-5-carbonitrile (350A-2)

To a solution of 5-bromo-7-methylquinoline (1.00 g, 4.50 mmol, 1.0 eq) in DMF (30 mL) were added Zn(CN)₂ (1.59 g, 13.5 mmol, 857 μL, 3.0 eq) BrettPhos Pd G3 (816 mg, 900 μmol, 0.2 eq), and BrettPhos (483 mg, 900 μmol, 0.2 eq). The mixture was stirred at 80° C. for 1 h. LCMS indicated that the starting material was completely consumed, and the desired mass was detected. The reaction mixture was allowed to cool to room temperature, poured into H₂O (40 mL) and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 0/1 to 1/2. 7-Methylquinoline-5-carbonitrile (600 mg, 3.57 mmol, 79% yield) was obtained as a white solid. M+H⁺=169.2 (LCMS).

### Step 3: 1-(7-Methylquinolin-5-yl)cyclopropanamine (350A-3)

A mixture of 7-methylquinoline-5-carbonitrile (180 mg, 1.07 mmol, 1.0 eq) in anhydrous Et₂O (20 mL) was degassed and purged with N₂ three times. The mixture was stirred with a mechanical stirrer at −78° C. To this mixture was added Ti(i-PrO)₄ (456 mg, 1.61 mmol, 473 μL, 1.5 eq) slowly, and then EtMgBr (3 M in Et₂O, 784 μL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. BF₃·Et₂O (304 mg, 2.14 mmol, 264 μL, 2.0 eq) was added slowly with no obvious temperature change. The mixture was stirred at 20° C. for another 1 h. TLC indicated that the starting material was completely consumed, and a main spot was detected. The reaction mixture was added into a mixture of HCl (1 M aqueous) (5.0 mL) and MTBE (5.0 mL) and extracted with MTBE (5.0 mL×2). The aqueous layer was basified to pH 8 by using NaOH (2 M aqueous), and a precipitate was formed. The mixture was filtered through a pad of Celite and the slurry was washed with DCM several times. The combined filtrate was extracted with DCM (5.0 mL×4). The combined organic layers were dried over Na₂SO₄, filtered, and concentrated under vacuum to give a residue which was purified by flash silica gel chromatography using a gradient of EtOAc/petroleum ether from 0/1 to 1/1. 1-(7-Methylquinolin-5-yl)cyclopropanamine (220 mg, crude) was obtained as a white solid. M+H⁺=199.0 (LCMS).

### Step 4: (S)-2-Methyl-5-((1-methylazetidin-2-yl)methoxy)-N-(1-(7-methylquinolin-5-yl)cyclopropyl)benzamide (Compound 581)

A mixture of 1-(7-methylquinolin-5-yl)cyclopropanamine (20.0 mg, 101 μmol, 1.0 eq), (S)-2-methyl-5-((1-methylazetidin-2-yl)methoxy)benzoic acid (23.7 mg, 101 μmol, 1.0 eq), HATU (76.7 mg, 202 μmol, 2.0 eq), and DIEA (39.1 mg, 303 μmol, 52.7 μL, 3.0 eq) in DMF (2.0 mL) was stirred at 20° C. for 2 h. LCMS indicated that the starting material was completely consumed, and the desired mass was detected. The mixture was poured into H₂O (4.0 mL) and extracted with EtOAc (2.0 mL×2), dried over Na₂SO₄ filtered and concentrated under vacuum to give a residue which was purified by preparative HPLC (Waters Xbridge BEH C18 column (100×30 mm, 10 μm); flow rate: 25 mL/min; gradient: 20%-50% B over 8 min; mobile phase A: 10 mM aqueous NH₄HCO₃, mobile phase B: acetonitrile). (S)-2-Methyl-5-((1-methylazetidin-2-yl)methoxy)-N-(1-(7-methylquinolin-5-yl)cyclopropyl)benzamide (10.0 mg, 21.7 μmol, 22% yield) was obtained as a white solid. M+H⁺=416.1 (LCMS); ¹H NMR (400 MHz, DMSO-d₆) δ 9.08 (s, 1H), 9.01 (d, J=8.4 Hz, 1H), 8.85 (d, J=4.1 Hz, 1H), 7.72 (d, J=6.9 Hz, 2H), 7.50 (dd, J=4.3, 8.5 Hz, 1H), 7.03 (d, J=8.5 Hz, 1H), 6.83 (dd, J=2.6, 8.4 Hz, 1H), 6.59 (d, J=2.6 Hz, 1H), 3.86 (d, J=5.5 Hz, 2H), 3.30 (s, 3H), 3.26-3.17 (m, 2H), 2.75-2.67 (m, 1H), 2.20 (s, 3H), 1.98-1.91 (m, 4H), 1.89-1.78 (m, 1H), 1.40-1.30 (m, 2H), 1.19 (br s, 2H).

### Example 351: (S)-2-Methyl-5-((1-methylazetidin-2-yl)methoxy)-N-(1-(7-(trifluoromethyl)quinolin-5-yl)cyclopropyl)benzamide (Compound 600)

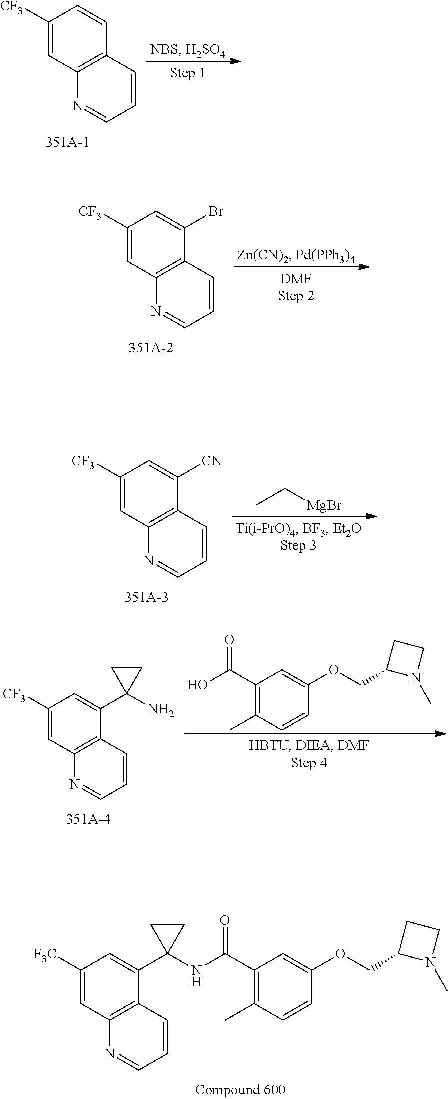

### Step 1: 5-Bromo-7-(trifluoromethyl)quinoline (351A-2)

To a solution of 7-(trifluoromethyl)quinoline (900 mg, 4.56 mmol, 1.0 eq) in H₂SO₄ (9.0 mL, 98% purity) was added NBS (1.62 g, 9.13 mmol, 2.0 eq) at 70° C. The mixture was stirred at 70° C. for 3.5 h. LCMS indicated that the starting material was completely consumed. The reaction mixture was allowed to cool to room temperature and then quenched by saturated aqueous NaHCO₃ (20 mL) and extracted with EtOAc (10 mL×3). The combined organic layers were washed with brine (10 mL×3), dried over Na₂SO₄, filtered, and concentrated under vacuum to give a residue which was purified by flash silica gel chromatography using a gradient of EtOAc/petroleum ether from 0/1 to 1/9. 5-Bromo-7-(trifluoromethyl)quinoline (735 mg, 2.50 mmol, 93% yield) was obtained as a white solid. M+H⁺=276.0 (LCMS).

### Step 2: 7-(Trifluoromethyl)quinoline-5-carbonitrile (351A-3)

To a solution of 5-bromo-7-(trifluoromethyl)quinoline (735 mg, 2.66 mmol, 1.0 eq) in DMF (10 mL) were added Pd(PPh₃)₄ (307 mg, 266 μmol, 0.1 eq) and Zn(CN)₂ (712 mg, 6.07 mmol, 2.3 eq) at 20° C. The resulting mixture was stirred at 120° C. for 16 h under a N₂ atmosphere. LCMS indicated that the starting material was completely consumed. The reaction mixture was allowed to cool to room temperature. The mixture was treated with H₂O (50 mL) and extracted with EtOAc (25 mL×3). The combined organic layers were dried over Na₂SO₄, filtered, and concentrated under vacuum to give a residue which was purified by flash silica gel chromatography using a gradient of EtOAc/petroleum ether from 0/1 to 0/1. 7-(Trifluoromethyl)quinoline-5-carbonitrile (528 mg, 2.31 mmol, 86% yield) was obtained as a white solid. M+H⁺=223.1 (LCMS).

### Step 3: 1-(7-(Trifluoromethyl)quinolin-5-yl)cyclopropanamine (351A-4)

A mixture of 7-(trifluoromethyl)quinoline-5-carbonitrile (50.0 mg, 225 μmol, 1.0 eq) in anhydrous Et₂O (10 mL) was degassed and purged with N₂ three times. The mixture was stirred at −78° C. To this mixture was added Ti(i-PrO)₄ (95.9 mg, 337 μmol, 99.6 mL, 1.5 eq) slowly, and then EtMgBr (3 M in Et₂O, 165 mL, 2.2 eq) was added dropwise to maintain the temperature between −78° C. and −75° C. over 1 h under a N₂ atmosphere. After the addition was complete, the resulting mixture was stirred at the same temperature for 10 min and warmed to 20° C. over 1 h. BF₃·Et₂O (63.8 mg, 450 μmol, 55.5 mL, 2.0 eq) was added slowly with no obvious temperature change. The mixture was stirred at 20° C. for another 1 h. TLC indicated that the starting material was completely consumed, and a main spot was detected. The reaction mixture was added into a mixture of HCl (1 M aqueous) (5.0 mL) and MTBE (5.0 mL) and extracted with MTBE (5.0 mL×2). The aqueous layer was basified to pH 8 by using NaOH (2 M aqueous), and a precipitate was formed. The mixture was filtered through a pad of Celite and the slurry was washed with DCM several times. The combined filtrate was extracted with DCM (5.0 mL×3). The combined organic layers were dried over Na₂SO₄, filtered, and concentrated under vacuum to give a residue which was purified by preparative TLC (DCM/MeOH=10/1, R_{f}=0.55). 1-(7-(Trifluoromethyl)quinolin-5-yl)cyclopropanamine (10.0 mg, 37.0 μmol, 16% yield) was obtained as a yellow oil. M+H⁺=253.1 (LCMS).

### Step 4: (S)-2-Methyl-5-((1-methylazetidin-2-yl)methoxy)-N-(1-(7-(trifluoromethyl)quinolin-5-yl)cyclopropyl)benzamide (Compound 600)

To a solution of(S)-2-methyl-5-((1-methylazetidin-2-yl)methoxy)benzoic acid (11.3 mg, 41.0 μmol, 1.2 eq, HCl salt) and 1-(7-(trifluoromethyl)quinolin-5-yl)cyclopropanamine (10.0 mg, 34.6 μmol, 1.0 eq, HCl salt) in DMF (1.0 mL) were added HBTU (32.8 mg, 86.6 μmol, 2.5 eq) and DIEA (13.4 mg, 103 μmol, 18.1 μL, 3.0 eq). The mixture was stirred at 20° C. for 2 h. LCMS indicated that the starting material was completely consumed, and the desired mass was detected. The reaction mixture was filtered and the filtrate was purified by preparative HPLC (Phenomenex Gemini C18 column (150×40 mm, 10 μm); flow rate: 60 mL/min; gradient: 5%-45% B over 8 min; mobile phase A: 0.04% aqueous HCl, mobile phase B: acetonitrile). (S)-2-Methyl-5-((1-methylazetidin-2-yl)methoxy)-N-(1-(7-(trifluoromethyl)quinolin-5-yl)cyclopropyl)benzamide (6.90 mg, 99.7 μmol, 39% yield) was obtained as a yellow solid. M+H⁺=470.2 (LCMS); ¹H NMR (400 MHZ, DMSO-d₆) δ10.55-10.38 (m, 1H), 9.29 (s, 1H), 9.24 (br d, J=8.6 Hz, 1H), 9.11 (dd, J=1.4, 4.1 Hz, 1H), 8.34 (s, 1H), 8.09 (d, J=1.6 Hz, 1H), 7.83 (dd, J=4.3, 8.6 Hz, 1H), 7.09 (d, J=8.4 Hz, 1H), 6.92 (dd, J=2.7, 8.3 Hz, 1H), 6.72 (d, J=2.6 Hz, 1H), 4.66-4.56 (m, 1H), 4.35-4.28 (m, 1H), 4.25-4.18 (m, 1H), 3.85-3.80 (m, 2H), 2.90-2.85 (m, 1H), 2.81 (d, J=4.9 Hz, 3H), 2.44 (br d, J=5.5 Hz, 1H), 1.94 (s, 3H), 1.42 (br s, 2H), 1.31 (br s, 2H).

### Example 352: (S)-2-Methyl-5-((1-methylazetidin-2-yl)methoxy)-N-(1-(7-(prop-1-en-2-yl) quinolin-5-yl)cyclopropyl)benzamide (Compound 568)

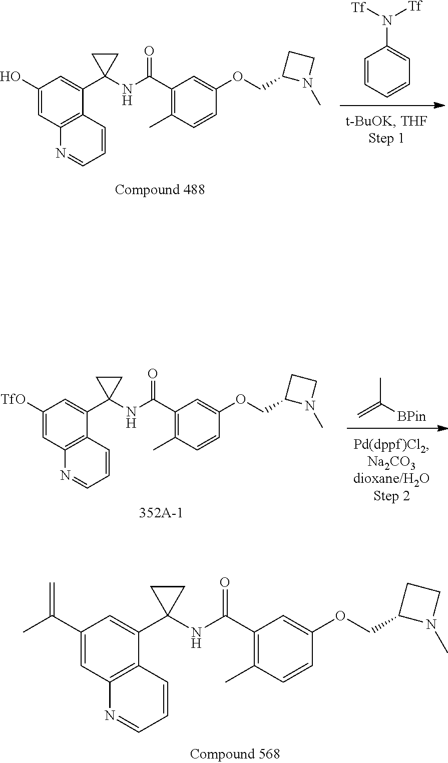

### Step 1: (S)-5-(1-(2-Methyl-5-((1-methylazetidin-2-yl)methoxy)benzamido)cyclopropyl)quinolin-7-yl trifluoromethanesulfonate (352A-1)

To a solution of(S)—N-(1-(7-hydroxyquinolin-5-yl)cyclopropyl)-2-methyl-5-((1-methylazetidin-2-yl)methoxy)benzamide (450 mg, 1.08 mmol, 1.0 eq) in THF (27 mL) was added 1-BuOK (242 mg, 2.16 mmol, 2.0 eq) at 0° C. The mixture was stirred at 0° C. for 30 min. 1,1,1-Trifluoro-N-phenyl-N-((trifluoromethyl)sulfonyl)methanesulfonamide (770 mg, 2.16 mmol, 2.0 eq) was added. The resulting mixture was stirred at 20° C. for 16 h. LCMS indicated that the starting material was completely consumed, and the desired mass was detected. The mixture was poured into H₂O (10 mL) and extracted with DCM (10 mL×5). The combined organic layers were dried over Na₂SO₄, filtered, and concentrated under vacuum to give a residue which was purified by flash silica gel chromatography using a gradient of EtOAc/petroleum ether from 0/1 to 1/0. (S)-5-(1-(2-Methyl-5-((1-methylazetidin-2-yl)methoxy)benzamido)cyclopropyl)quinolin-7-yl trifluoromethanesulfonate (370 mg, 673 μmol, 31% yield) was obtained as a yellow solid. M+H⁺=550.2 (LCMS); ¹H NMR (400 MHz, DMSO-d₆) δ 9.21 (s, 1H), 9.14 (d, J=8.5 Hz, 1H), 9.05 (dd, J=1.5, 4.3 Hz, 1H), 8.08 (d, J=2.5 Hz, 1H), 7.88 (d, J=2.5 Hz, 1H), 7.74 (dd, J=4.2, 8.7 Hz, 1H), 7.03 (d, J=8.5 Hz, 1H), 6.85 (dd, J=2.7, 8.3 Hz, 1H), 6.64 (d, J=2.8 Hz, 1H), 3.89 (d, J=5.4 Hz, 2H), 3.29-3.25 (m, 1H), 2.84-2.73 (m, 1H), 2.24 (s, 3H), 1.99 (s, 1H), 1.98-1.94 (m, 1H), 1.92 (s, 3H), 1.90-1.81 (m, 1H), 1.44-1.38 (m, 2H), 1.30-1.25 (m, 2H).

### Step 2: (S)-2-Methyl-5-((1-methylazetidin-2-yl)methoxy)-N-(1-(7-(prop-1-en-2-yl) quinolin-5-yl)cyclopropyl)benzamide (Compound 568)

To a solution of(S)-5-(1-(2-methyl-5-((1-methylazetidin-2-yl)methoxy)benzamido)cyclopropyl)quinolin-7-yl trifluoromethanesulfonate (70.0 mg, 127 μmol, 1.0 eq) and 2-isopropenyl-4,4,5,5-tetramethyl-1,3,2-dioxaborolane (23.5 mg, 140 μmol, 1.1 eq) in a mixture of dioxane (5.0 mL) and H₂O (1.0 mL) were added Na₂CO₃ (31.0 mg, 293 μmol, 2.3 eq) and Pd(dppf)Cl₂ (10.4 mg, 12.7 μmol, 0.1 eq). The mixture was stirred at 80° C. for 16 h under a N₂ atmosphere. LCMS indicated that the starting material was completely consumed, and the desired mass was detected. The reaction mixture was allowed to cool to room temperature, poured into water (10 mL) and extracted with DCM (5.0 mL×4). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 1%-15% B over 8 min; mobile phase A: 0.04% aqueous HCl, mobile phase B: acetonitrile). (S)-2-Methyl-5-((1-methylazetidin-2-yl)methoxy)-N-(1-(7-(prop-1-en-2-yl)quinolin-5-yl)cyclopropyl)benzamide (28.0 mg, 54.0 μmol, 43% yield, HCl salt) was obtained as a yellow solid. M+H⁺=442.1 (LCMS); ¹H NMR (400 MHZ, DMSO-d₆) δ 9.65 (br d, J=8.5 Hz, 1H), 9.33 (s, 1H), 9.21 (br d, J=4.8 Hz, 1H), 8.31 (s, 1H), 8.19 (s, 1H), 8.03 (dd, J=5.1, 8.4 Hz, 1H), 7.09 (br d, J=8.4 Hz, 1H), 6.91 (br dd, J=2.4, 8.4 Hz, 1H), 6.74 (d, J=2.3 Hz, 1H), 5.87 (s, 1H), 5.51 (s, 1H), 4.70-4.56 (m, 1H), 4.38-4.27 (m, 1H), 4.26-4.15 (m, 1H), 4.08-3.95 (m, 1H), 3.91-3.77 (m, 1H), 2.81 (s, 3H), 2.69 (s, 1H), 2.34-2.31 (m, 1H), 2.27 (s, 3H), 1.96 (s, 3H), 1.44 (br s, 2H), 1.34 (br s, 2H).

### Example 353: (S)-2-Methyl-5-((1-methylazetidin-2-yl)methoxy)-N-(1-(7-(thiophen-2-yl)quinolin-5-yl)cyclopropyl)benzamide (Compound 551)

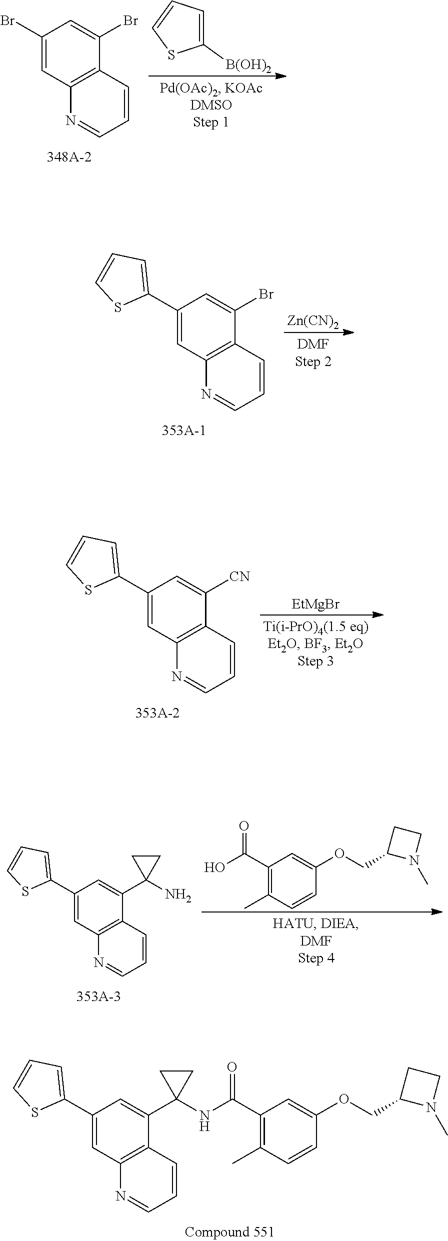

### Step 1: 5-Bromo-7-(thiophen-2-yl)quinoline (353A-1)

To a mixture of 5,7-dibromoquinoline (2.00 g, 6.97 mmol, 1.0 eq) and thiophen-2-ylboronic acid (713 mg, 5.58 mmol, 0.8 eq) in DMSO (100 mL) were added KOAc (2.74 g, 27.9 mmol, 4.0 eq), cataCXium A (1.25 g, 3.48 mmol, 0.5 eq) and Pd(OAc) 2 (313 mg, 1.39 mmol, 0.2 eq). The mixture was degassed and purged with N₂ for 3 times, then stirred at 80° C. for 16 h under a N₂ atmosphere. LCMS indicated that the starting material was completely consumed. The reaction mixture was allowed to cool to room temperature, poured into H₂O (100 mL) and extracted with EtOAc (50 mL×4). The combined organic layers were washed with brine (50 mL×2), dried over Na₂SO₄, filtered, and concentrated under vacuum to give a residue which was purified by flash silica gel chromatography using a gradient of EtOAc/petroleum ether from 0/1 to 1/10. 5-Bromo-7-(thiophen-2-yl)quinoline (220 mg, 758 μmol, 11% yield) was obtained as a yellow solid. M+H⁺=289.9 (LCMS); ¹H NMR (400 MHZ, DMSO-d₆) δ 8.98 (dd, J=1.5, 4.3 Hz, 1H), 8.48 (d, J=8.7 Hz, 1H), 8.35 (d, J=1.6 Hz, 1H), 8.25 (s, 1H), 7.89 (dd, J=0.9, 3.6 Hz, 1H), 7.77-7.63 (m, 2H), 7.23 (dd, J=3.8, 5.0 Hz, 1H).

### Step 2: 7-(Thiophen-2-yl)quinoline-5-carbonitrile (353A-2)

To a mixture of 5-bromo-7-(thiophen-2-yl)quinoline (160 mg, 551 μmol, 1.0 eq) in DMF (8.0 mL) were added Zn(CN)₂ (130 mg, 1.10 mmol, 70.0 μL, 2.0 eq), BrettPhos Pd G3 (100 mg, 110 μmol, 0.2 eq), BrettPhos (59.1 mg, 110 μmol, 0.2 eq). The mixture was stirred at 80° C. for 1 h under a N₂ atmosphere. LCMS indicated that the starting material was completely consumed. The reaction mixture was allowed to cool to room temperature, poured into H₂O (5.0 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/0. 7-(Thiophen-2-yl)quinoline-5-carbonitrile (100 mg, 422 μmol 89% yield) was obtained as a yellow solid. ¹H NMR (400 MHZ, DMSO-d₆) δ 9.09 (dd, J=1.6, 4.2 Hz, 1H), 8.71 (d, J=1.9 Hz, 1H), 8.54-8.42 (m, 2H), 7.95 (dd, J=0.9, 3.6 Hz, 1H), 7.82-7.70 (m, 2H), 7.26 (dd, J=3.8, 5.0 Hz, 1H).

### Step 3: 1-(7-(Thiophen-2-yl)quinolin-5-yl)cyclopropanamine (353A-3)

A mixture of 7-(thiophen-2-yl)quinoline-5-carbonitrile (100 mg, 423 μmol, 1.0 eq) in anhydrous Et₂O (20 mL) was degassed and purged with N₂ three times. The mixture was cooled to −78° C. To this mixture was added Ti(i-PrO)₄ (180 mg, 635 μmol, 187 μL, 1.5 eq) slowly, and then EtMgBr (3 M, 310 μL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 2 h. BF₃·Et₂O (120 mg, 846 μmol, 104 μL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (5.0 mL) and MTBE (5.0 mL) and extracted with MTBE (8.0 mL×2). The aqueous layer was basified to pH 8 by using NaOH (2 M aqueous), and a precipitate was formed. The mixture was filtered through a pad of Celite and the slurry was washed with DCM several times. The combined filtrate was extracted with DCM (10 mL×3). The combined organic layers were dried over Na₂SO₄, filtered, and concentrated under vacuum to give a residue which was purified by preparative TLC (DCM/MeOH=10/1, R_{f}=0.24). 1-(7-(Thiophen-2-yl)quinolin-5-yl)cyclopropanamine (25.0 mg, 93.9 μmol, 22% yield) was obtained as a yellow gum. ¹H NMR (400 MHZ, CDCl₃) δ 8.97 (br d, J=2.7 Hz, 1H), 8.79 (br d, J=8.2 Hz, 1H), 8.29 (s, 1H), 7.89 (s, 1H), 7.56 (d, J=4.0 Hz, 1H), 7.49 (br dd, J=4.1, 8.6 Hz, 1H), 7.42 (d, J=4.8 Hz, 1H), 7.19 (dd, J=3.7, 5.0 Hz, 1H), 1.31-1.18 (m, 4H).

### Step 4: (S)-2-Methyl-5-((1-methylazetidin-2-yl)methoxy)-N-(1-(7-(thiophen-2-yl)quinolin-5-yl)cyclopropyl)benzamide (Compound 551)

To a solution of(S)-2-methyl-5-((1-methylazetidin-2-yl)methoxy)benzoic acid (20.0 mg, 85.0 μmol, 1.0 eq) and 1-(7-(thiophen-2-yl)quinolin-5-yl)cyclopropanamine (22.6 mg, 85.0 μmol, 1.0 eq) in DMF (2.0 mL) were added HATU (80.8 mg, 213 μmol, 2.5 eq) and DIEA (33.0 mg, 255 μmol, 44.4 μL, 3.0 eq). The mixture was stirred at 20° C. for 16 h. LCMS indicated that the starting material was completely consumed, and the desired mass was detected. The reaction mixture was poured into H₂O (30 mL) and extracted with EtOAc (20 mL×3). The combined organic layers were dried over Na₂SO₄, filtered, and concentrated under vacuum to give a residue which was purified by preparative HPLC (Waters Xbridge BEH C18 (100×30 mm, 10 μm); flow rate: 25 mL/min; gradient: 30%-60% B over 8 min; 10 mM aqueous NH₄HCO₃, mobile phase B: acetonitrile). (S)-2-Methyl-5-((1-methylazetidin-2-yl)methoxy)-N-(1-(7-(thiophen-2-yl)quinolin-5-yl)cyclopropyl)benzamide (8.70 mg, 17.4 μmol, 21% yield) was obtained as a yellow solid. M+H⁺=484.1 (LCMS); ¹H NMR (400 MHZ, CDCl₃) δ 9.02 (d, J=8.6 Hz, 1H), 8.94 (dd, J=1.4, 4.1 Hz, 1H), 8.29 (s, 1H), 8.22 (d, J=1.8 Hz, 1H), 7.58 (d, J=3.6 Hz, 1H), 7.47 (dd, J=4.1, 8.5 Hz, 1H), 7.38 (d, J=5.0 Hz, 1H), 7.16 (dd, J=3.8, 4.9 Hz, 1H), 7.04-6.97 (m, 1H), 6.82-6.76 (m, 1H), 6.71 (d, J=2.4 Hz, 1H), 6.51-6.37 (m, 1H), 3.94-3.83 (m, 2H), 3.44 (br d, J=4.1 Hz, 1H), 3.37-3.26 (m, 1H), 2.84 (q, J=7.6 Hz, 1H), 2.35 (s, 3H), 2.14 (s, 3H), 2.08-1.98 (m, 2H), 1.67-1.64 (m, 2H), 1.46-1.41 (m, 2H).

### Example 354: (S)-2-Methyl-5-((1-methylazetidin-2-yl)methoxy)-N-(1-(7-(oxazol-2-yl)quinolin-5-yl)cyclopropyl)benzamide (Compound 588)

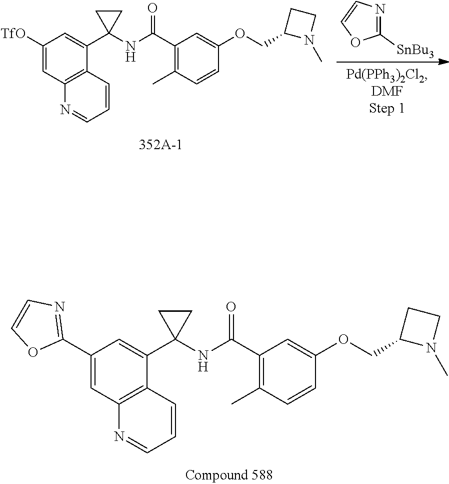

### Step 1: (S)-2-Methyl-5-((1-methylazetidin-2-yl)methoxy)-N-(1-(7-(oxazol-2-yl)quinolin-5-yl)cyclopropyl)benzamide (Compound 588)

To a solution of(S)-5-(1-(2-methyl-5-((1-methylazetidin-2-yl)methoxy)benzamido)cyclopropyl)quinolin-7-yltrifluoromethanesulfonate (65.0 mg, 118 μmol, 1.0 eq) and 2-(tributylstannyl)oxazole (84.7 mg, 237 μmol, 2.0 eq) in DMF (5.0 mL) was added Pd(PPh₃)₂Cl₂ (8.30 mg, 11.8 μmol, 0.1 eq). The mixture was degassed and purged with N₂ three times, and then the mixture was stirred at 60° C. for 14 h under a N₂ atmosphere. LCMS indicated that the starting material was completely consumed, and the desired mass was detected. The reaction mixture was allowed to cool to room temperature and filtered. The filtrate was purified by preparative HPLC (Phenomenex luna C18 column (80× 40 mm, 3 μm); flow rate: 40 mL/min; gradient: 5%-35% B over 7 min; mobile phase A: 0.04% aqueous HCl, mobile phase B: acetonitrile). (S)-2-Methyl-5-((1-methylazetidin-2-yl)methoxy)-N-(1-(7-(oxazol-2-yl)quinolin-5-yl)cyclo propyl)benzamide (12.1 mg, 24.8 μmol, 21% yield, HCl salt) was obtained as a white solid. M+H⁺=469.1 (LCMS); ¹H NMR (400 MHZ, DMSO-d₆) δ 9.34 (d, J=8.5 Hz, 1H), 9.28 (s, 1H), 9.12 (dd, J=1.4, 4.5 Hz, 1H), 8.62-8.52 (m, 2H), 8.37 (s, 1H), 7.85 (dd, J=4.6, 8.6 Hz, 1H), 7.53 (s, 1H), 7.14-7.06 (m, 1H), 6.99-6.88 (m, 1H), 6.80-6.69 (m, 1H), 4.73-4.52 (m, 1H), 4.32-4.17 (m, 2H), 4.01 (dt, J=4.8, 9.6 Hz, 1H), 3.85 (q, J=9.4 Hz, 1H), 2.82 (s, 3H), 2.40-2.26 (m, 2H), 1.94 (s, 3H), 1.45 (br s, 2H), 1.31 (br s, 2H).

### Example 355: N-(1-(2-Chloroquinolin-5-yl)cyclopropyl)-2-methyl-5-((1-methylazetidin-2-yl)methoxy)benzamide (Compound 436)

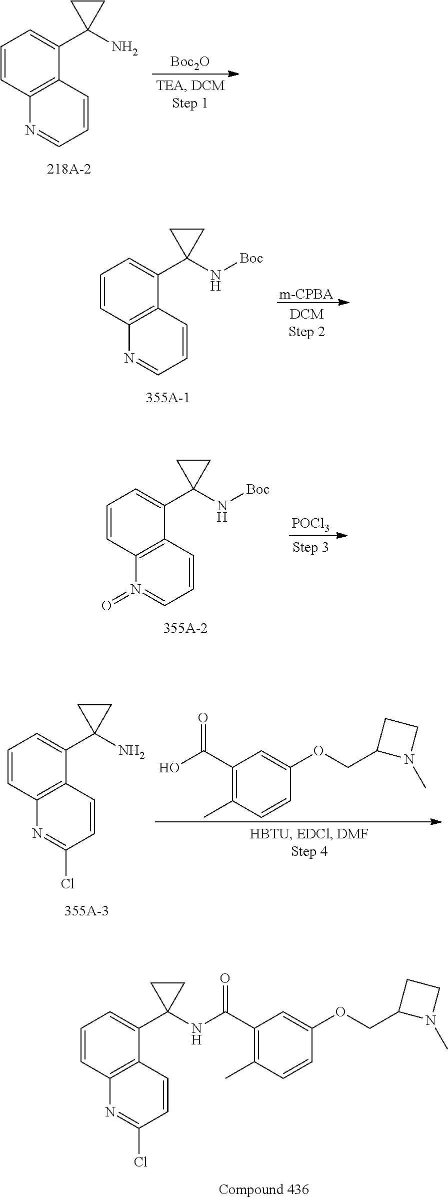

### Step 1: tert-Butyl(1-(quinolin-5-yl)cyclopropyl)carbamate (355A-1)

To a solution of 1-(quinolin-5-yl)cyclopropanamine (590 mg, 2.71 mmol, 1.0 eq) in DCM (40 mL) were added TEA (549 mg, 5.43 mmol, 755 μL, 2.0 eq) and Boc₂O (711 mg, 3.26 mmol, 748 μL, 1.2 eq). The mixture was stirred at 20° C. for 16 h. LCMS indicated that the starting material was completely consumed, and the desired mass was detected. The mixture was treated with H₂O (30 mL) and extracted with DCM (20 mL×3). The combined organic layers were dried over Na₂SO₄, filtered, and concentrated under vacuum to give a residue which was purified by flash silica gel chromatography using a gradient of EtOAc/petroleum ether from 0/1 to 1/4. tert-Butyl(1-(quinolin-5-yl)cyclopropyl)carbamate (590 mg, 2.07 mmol, 76% yield) was obtained as a white solid. M+H⁺=285.1 (LCMS).

### Step 2: 5-(1-((tert-Butoxycarbonyl)amino)cyclopropyl)quinoline 1-oxide (355A-2)

To a solution of tert-butyl(1-(quinolin-5-yl)cyclopropyl)carbamate (290 mg, 1.02 mmol, 1.0 eq) in DCM (20 mL) was added m-CPBA (311 mg, 1.53 mmol, 85% purity, 1.5 eq). The mixture was stirred at 20° C. for 16 h. LCMS indicated that the starting material was completely consumed, and the desired mass was detected. The mixture was treated with saturated aqueous NaHCO₃ (20 mL) and extracted with DCM (5.0 mL×4). The combined organic layers were dried over Na₂SO₄, filtered, and concentrated under vacuum to give a residue which was purified by flash silica gel chromatography using a gradient of DCM/MeOH from 0/1 to 1/5. 5-(1-((tert-Butoxycarbonyl)amino)cyclopropyl)quinoline 1-oxide (240 mg, 799 μmol, 78% yield) was obtained as a white solid. M+H⁺=301.1 (LCMS).

### Step 3: 1-(2-Chloroquinolin-5-yl)cyclopropanamine (355A-3)

A solution of 5-(1-((tert-butoxycarbonyl)amino)cyclopropyl)quinoline 1-oxide (410 mg, 1.37 mmol, 1.0 eq) in POCl₃ (6.60 g, 43.1 mmol, 4.00 mL, 31 eq) was stirred at 80° C. for 1 h. LCMS indicated that the starting material was completely consumed, and the desired mass was detected. The reaction mixture was allowed to cool to room temperature, concentrated under vacuum to remove POCl₃, the residue was diluted with H₂O (5.0 mL), basified to pH 7 by using NH₃·H₂O (25% purity), and extracted with DCM (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 3/5. 1-(2-Chloroquinolin-5-yl)cyclopropanamine (150 mg, 686 μmol, 50% yield) was obtained as a white solid. M+H⁺=219.0 (LCMS).

### Step 4: N-(1-(2-Chloroquinolin-5-yl)cyclopropyl)-2-methyl-5-((1-methylazetidin-2-yl)methoxy)benzamide (Compound 436)

To a solution of 1-(2-chloroquinolin-5-yl)cyclopropanamine (50.0 mg, 229 μmol, 1.0 eq) and 2-methyl-5-((1-methylazetidin-2-yl)methoxy)benzoic acid (53.8 mg, 229 μmol, 1.0 eq) in DMF (5.0 mL) were added DIEA (δ8.6 mg, 686 μmol, 119 μL, 3.0 eq) and HBTU (173 mg, 457 μmol, 2.0 eq). The mixture was stirred at 20° C. for 16 h. LCMS indicated that the starting material was completely consumed, and the desired mass was detected. The mixture was treated with H₂O (5.0 mL) and extracted with EtOAc (5.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 25%-55% B over 8 min; mobile phase A: 0.1% aqueous TFA, mobile phase B: acetonitrile). N-(1-(2-Chloroquinolin-5-yl)cyclopropyl)-2-methyl-5-((1-methylazetidin-2-yl)methoxy)benzamide (12.1 mg, 21.7 μmol, 9% yield, TFA salt) was obtained as a white solid. M+H⁺=436.1 (LCMS); ¹H NMR (400 MHZ, DMSO-d₆)) δ 9.87 (br dd, J=1.8, 8.0 Hz, 1H), 9.24-9.05 (m, 2H), 7.91 (dd, J=7.7, 14.4 Hz, 2H), 7.82-7.74 (m, 1H), 7.69 (d, J=8.9 Hz, 1H), 7.10 (d, J=8.4 Hz, 1H), 6.92 (dd, J=2.6, 8.4 Hz, 1H), 6.71 (d, J=2.6 Hz, 1H), 4.61 (br d, J=4.0 Hz, 1H), 4.33-4.16 (m, 2H), 4.07-3.98 (m, 1H), 3.91-3.85 (m, 1H), 2.84 (d, J=4.8 Hz, 3H), 2.42-2.20 (m, 2H), 1.95 (s, 3H), 1.36 (br s, 2H), 1.29-1.19 (m, 2H).

### Example 356: N-(1-(2-Chloroquinolin-5-yl)cyclopropyl)-2-methyl-5-((1-methylazetidin-2-yl)methoxy)benzamide (Compound 529)

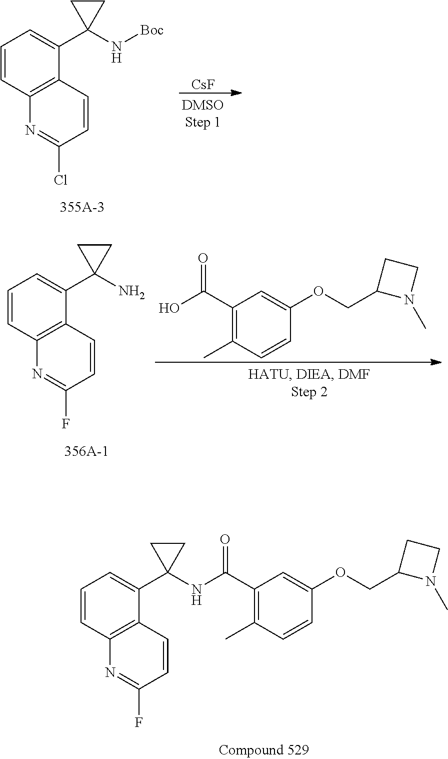 [SMILES: CC(C)(C)OC(=O)NC1(c2cccc3nc(Cl)ccc23)CC1 ; "CsF" ; NC1(c2cccc3nc(F)ccc23)CC1 ; Cc1ccc(OCC2CCN2C)cc1C(=O)O ; Cc1ccc(OCC2CCN2C)cc1C(=O)NC1(c2cccc3nc(F)ccc23)CC1]

### Step 1: 1-(2-Fluoroquinolin-5-yl)cyclopropanamine (356A-1)

To a solution of tert-butyl(1-(2-chloroquinolin-5-yl)cyclopropyl)carbamate (130 mg, 408 μmol, 1.0 eq) in DMSO (5.0 mL) was added CsF (92.9 mg, 612 μmol, 22.5 μL, 1.5 eq). The mixture was stirred at 140° C. for 5 h. LCMS indicated that the starting material was completely consumed, and the desired mass was detected. The reaction mixture was allowed to cool to room temperature, poured into H₂O (5.0 mL) and extracted with EtOAc (3.0 mL×3). The combined organic layers were dried over Na₂SO₄, filtered, and concentrated under vacuum to give a residue which was purified by preparative TLC (DCM/MeOH=10/1, R_{f}=0.1). 1-(2-Fluoroquinolin-5-yl)cyclopropanamine (35.0 mg, 173 μmol, 42% yield) was obtained as a white solid. M+H⁺=203.2 (LCMS); ¹H NMR (400 MHZ, DMSO-d₆) δ 9.14-9.03 (m, 1H), 7.78-7.73 (m, 1H), 7.71 (d, J=7.1 Hz, 1H), 7.58-7.53 (m, 1H), 7.40 (dd, J=2.9, 9.0 Hz, 1H), 1.04 (d, J=2.3 Hz, 2H), 0.95-0.79 (m, 2H).

### Step 2: N-(1-(2-Fluoroquinolin-5-yl)cyclopropyl)-2-methyl-5-((1-methylazetidin-2-yl)methoxy)benzamide (Compound 529)

To a solution of 1-(2-fluoroquinolin-5-yl)cyclopropanamine (20.0 mg, 98.9 μmol, 1.0 eq) and 2-methyl-5-((1-methylazetidin-2-yl)methoxy)benzoic acid (23.3 mg, 98.9 μmol, 1.0 eq) in DMF (2.0 mL) were added DIEA (38.4 mg, 297 μmol, 51.7 μL, 3.0 eq) and HATU (75.2 mg, 198 μmol, 2.0 eq). The mixture was stirred at 20° C. for 2 h. LCMS indicated that the starting material was completely consumed, and the desired mass was detected. The mixture was treated with H₂O (2.0 mL) and extracted with EtOAc (2.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 30%-70% B over 8 min; mobile phase A: 10 mM aqueous NH₄HCO₃ with NH₃·H₂O (0.05%), mobile phase B: acetonitrile). N-(1-(2-Fluoroquinolin-5-yl)cyclopropyl)-2-methyl-5-((1-methylazetidin-2-yl)methoxy)benzamide (6.00 mg, 14.5 μmol, 15% yield) was obtained as a yellow solid. M+H⁺=420.1 (LCMS); ¹H NMR (400 MHz, DMSO-d₆) δ 9.29 (t, J=8.8 Hz, 1H), 9.13 (s, 1H), 7.96-7.86 (m, 1H), 7.85-7.72 (m, 2H), 7.45 (dd, J=2.6, 9.0 Hz, 1H), 7.04 (d, J=8.5 Hz, 1H), 6.84 (dd, J=2.8, 8.4 Hz, 1H), 6.63 (d, J=2.6 Hz, 1H), 3.87 (d, J=5.4 Hz, 2H), 3.24 (br dd, J=2.3, 6.2 Hz, 2H), 2.80-2.62 (m, 1H), 2.21 (s, 3H), 1.95 (s, 5H), 1.49-1.30 (m, 2H), 1.29-1.16 (m, 2H).

### Example 357: (S)-2-Methyl-5-((1-methylazetidin-2-yl)methoxy)-N-(1-(2-methylquinolin-5-yl)cyclopropyl)benzamide (Compound 547)

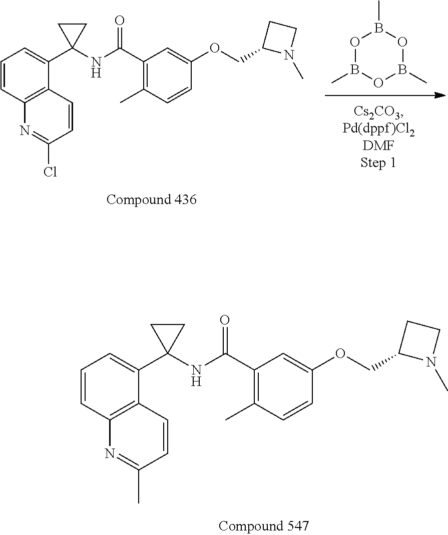

### Step 1: (S)-2-Methyl-5-((1-methylazetidin-2-yl)methoxy)-N-(1-(2-methylquinolin-5-yl)cyclopropyl)benzamide (Compound 547)

To a solution of(S)—N-(1-(2-chloroquinolin-5-yl)cyclopropyl)-2-methyl-5-((1-methylazetidin-2-yl)methoxy)benzamide (80.0 mg, 184 μmol, 1.0 eq) in DMF (4.0 mL) were added 2,4,6-trimethyl-1,3,5,2,4,6-trioxatriborinane (185 mg, 734 μmol, 205 μL, 50% purity in THF, 4.0 eq), Cs₂CO₃ (197 mg, 606 μmol, 3.3 eq) and Pd(dppf)Cl₂ (44.9 mg, 55.1 μmol, 0.3 eq). The mixture was degassed and purged with N₂ three times. The mixture was stirred at 110° C. for 16 h under a N₂ atmosphere. LCMS indicated that the starting material was completely consumed, and the desired mass was detected. The reaction mixture was allowed to cool to room temperature, concentrated under vacuum to give a residue which was purified by preparative HPLC (Phenomenex Luna C18 column (75×30 mm, 3 μm); flow rate: 25 mL/min; gradient: 5%-40% B over 8 min; mobile phase A: 0.2% aqueous FA, mobile phase B: acetonitrile). (S)-2-Methyl-5-((1-methylazetidin-2-yl)methoxy)-N-(1-(2-methylquinolin-5-yl)cyclopropyl)benzamide (27.2 mg, 58.9 μmol, 32% yield, FA salt) was obtained as a white solid. M+H⁺=416.1 (LCMS); ¹H NMR (400 MHZ, DMSO-d₆) δ 9.10 (s, 1H), 8.97 (d, J=8.6 Hz, 1H), 8.18 (s, 1H), 7.94-7.74 (m, 2H), 7.69-7.59 (m, 1H), 7.48 (d, J=8.8 Hz, 1H), 7.04 (d, J=8.5 Hz, 1H), 6.85 (dd, J=2.6, 8.4 Hz, 1H), 6.62 (d, J=2.6 Hz, 1H), 3.95-3.86 (m, 2H), 3.43-3.31 (m, 2H), 2.87 (br d, J=7.9 Hz, 1H), 2.67 (s, 3H), 2.29 (s, 3H), 2.08-1.97 (m, 1H), 1.97-1.93 (m, 3H), 1.91 (s, 1H), 1.36 (s, 2H), 1.19 (br d, J=1.3 Hz, 2H).

### Example 358: (S)—N-(1-(2-Hydroxyquinolin-5-yl)cyclopropyl)-2-methyl-5-((1-methylazetidin-2-yl)methoxy)benzamide (Compound 545)

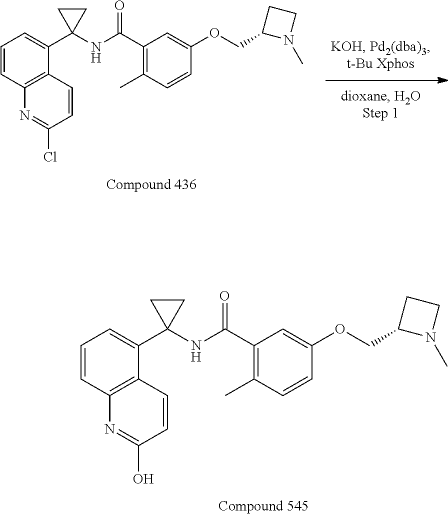 [SMILES: Cc1ccc(OC[C@@H]2CCN2C)cc1C(=O)NC1(c2cccc3nc(Cl)ccc23)CC1 ; Cc1ccc(OC[C@@H]2CCN2C)cc1C(=O)NC1(c2cccc3nc(O)ccc23)CC1]

### Step 1: (S)—N-(1-(2-Hydroxyquinolin-5-yl)cyclopropyl)-2-methyl-5-((1-methylazetidin-2-yl)methoxy)benzamide (Compound 545)

To a solution of(S)—N-(1-(2-chloroquinolin-5-yl)cyclopropyl)-2-methyl-5-((1-methylazetidin-2-yl)methoxy)benzamide (70.0 mg, 161 μmol, 1.0 eq) in a mixture of dioxane (7.0 mL) and H₂O (7.0 mL) were added KOH (180 mg, 3.21 mmol, 20 eq), Pd₂ (dba) 3 (2.94 mg, 3.21 μmol, 0.02 eq) and t-Bu Xphos (5.45 mg, 12.8 μmol, 0.08 eq). The mixture was degassed and purged with N₂ three times. The mixture was stirred at 80° C. for 16 h under a N₂ atmosphere. LCMS indicated that the starting material was completely consumed, and the desired mass was detected. The reaction mixture was allowed to cool to room temperature, poured into H₂O (15 mL) and extracted with DCM (5.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 1%-15% B over 8 min; mobile phase A: 0.2% aqueous FA, mobile phase B: acetonitrile). (S)—N-(1-(2-Hydroxyquinolin-5-yl)cyclopropyl)-2-methyl-5-((1-methylazetidin-2-yl)methoxy)benzamide (15.0 mg, 35.4 μmol, 22% yield, FA salt) was obtained as a white solid. M+H⁺=418.1 (LCMS); ¹H NMR (400 MHZ, DMSO-d₆) δ 11.74 (br s, 1H), 9.02 (s, 1H), 8.65 (d, J=9.9 Hz, 1H), 7.53-7.35 (m, 2H), 7.32-7.14 (m, 1H), 7.05 (d, J=8.5 Hz, 1H), 6.85 (dd, J=2.6, 8.3 Hz, 1H), 6.65 (d, J=2.6 Hz, 1H), 6.54 (d, J=9.9 Hz, 1H), 3.89 (d, J=5.4 Hz, 2H), 3.28-3.25 (m, 2H), 2.75 (br d, J=8.1 Hz, 1H), 2.24 (s, 3H), 2.01 (s, 3H), 1.98-1.74 (m, 2H), 1.28 (br s, 2H), 1.13 (br s, 2H).

### Example 359: (S)—N-(1-(2-Methoxyquinolin-5-yl)cyclopropyl)-2-methyl-5-((1-methylazetidin-2-yl)methoxy)benzamide (Compound 544)

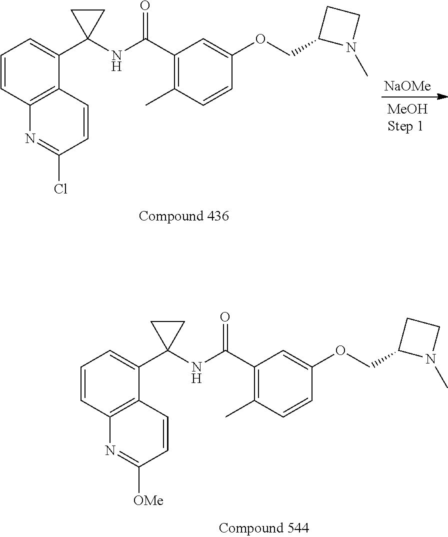 [SMILES: Cc1ccc(OC[C@@H]2CCN2C)cc1C(=O)NC1(c2cccc3nc(Cl)ccc23)CC1 ; COc1ccc2c(C3(NC(=O)c4cc(OC[C@@H]5CCN5C)ccc4C)CC3)cccc2n1 ; "NaOMe"]

### Step 1: (S)—N-(1-(2-Methoxyquinolin-5-yl)cyclopropyl)-2-methyl-5-((1-methylazetidin-2-yl)methoxy)benzamide (Compound 544)

To a solution of(S)—N-(1-(2-chloroquinolin-5-yl)cyclopropyl)-2-methyl-5-((1-methylazetidin-2-yl)methoxy)benzamide (70.0 mg, 161 μmol, 1.0 eq) in MeOH (4.0 mL) was added NaOMe (49.5 mg, 916 μmol, 4.0 eq). The mixture was stirred at 70° C. for 14 h. LCMS indicated that the starting material was completely consumed, and the desired mass was detected. The reaction mixture was allowed to cool to room temperature, concentrated under vacuum to give a residue which was purified by preparative HPLC (Phenomenex Luna C18 column (75× 30 mm, 3 μm); flow rate: 25 mL/min; gradient: 20%-55% B over 8 min; mobile phase A: 0.2% aqueous FA, mobile phase B: acetonitrile). (S)—N-(1-(2-Methoxyquinolin-5-yl)cyclopropyl)-2-methyl-5-((1-methylazetidin-2-yl)methoxy)benzamide (14.8 mg, 34.1 μmol, 21% yield, FA salt) was obtained as a white solid. M+H⁺=432.1 (LCMS); ¹H NMR (400 MHZ, DMSO-d₆) δ 9.07 (s, 1H), 8.97 (d, J=9.1 Hz, 1H), 7.74-7.65 (m, 2H), 7.64-7.51 (m, 1H), 7.05 (dd, J=8.8, 16.3 Hz, 2H), 6.83 (dd, J=2.6, 8.4 Hz, 1H), 6.61 (d, J=2.6 Hz, 1H), 3.98 (s, 3H), 3.87 (d, J=5.3 Hz, 1H), 3.28-3.11 (m, 3H), 2.73 (br d, J=8.3 Hz, 1H), 2.22 (s, 3H), 1.95 (s, 4H), 1.90-1.77 (m, 1H), 1.48-1.26 (m, 2H), 1.24-1.03 (m, 2H).

### Example 360: (S)—N-(1-(2-(Dimethylamino)quinolin-5-yl)cyclopropyl)-2-methyl-5-((1-methylazetidin-2-yl)methoxy)benzamide (Compound 548)

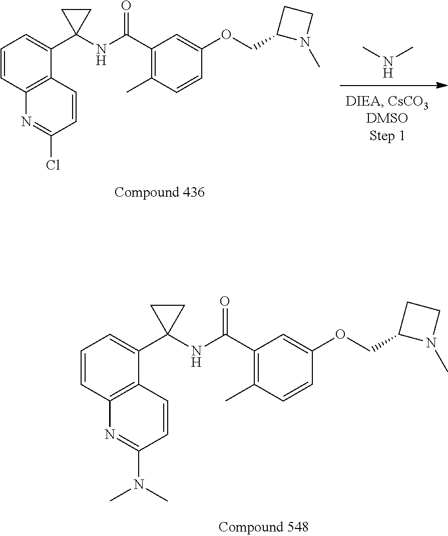

### Step 1: (S)—N-(1-(2-(Dimethylamino)quinolin-5-yl)cyclopropyl)-2-methyl-5-((1-methylazetidin-2-yl)methoxy)benzamide (Compound 548)

To a solution of(S)—N-(1-(2-chloroquinolin-5-yl)cyclopropyl)-2-methyl-5-((1-methylazetidin-2-yl)methoxy)benzamide (70.0 mg, 161 μmol, 1.0 eq) and dimethylamine (2 M in THF, 161 μmol, 2.0 eq) in DMSO (4.0 mL) were added DIEA (41.5 mg, 321 μmol, 55.9 μL, 2.0 eq) and CsF (48.8 mg, 321 μmol, 11.8 μL, 2.0 eq). The mixture was stirred at 120° C. for 5 h. LCMS indicated that the starting material was completely consumed, and the desired mass was detected. The reaction mixture was allowed to cool to room temperature, concentrated under vacuum to give a residue which was purified by preparative HPLC (Phenomenex Luna C18 column (75×30 mm, 3 μm); flow rate: 25 mL/min; gradient: 1%-25% B over 8 min; mobile phase A: 0.2% aqueous FA, mobile phase B: acetonitrile). (S)—N-(1-(2-(Dimethylamino)quinolin-5-yl)cyclopropyl)-2-methyl-5-((1-methylazetidin-2-yl)methoxy)benzamide (22.0 mg, 49.3 μmol, 31% yield, FA salt) was obtained as a white solid. M+H⁺=445.1 (LCMS); ¹H NMR (400 MHZ, DMSO-d₆) δ 9.02 (s, 1H), 8.77 (d, J=9.4 Hz, 1H), 8.16 (s, 2H), 7.59-7.34 (m, 3H), 7.16-6.99 (m, 2H), 6.84 (dd, J=2.6, 8.3 Hz, 1H), 6.63 (d, J=2.5 Hz, 1H), 3.95 (br d, J=4.4 Hz, 2H), 3.56-3.34 (m, 2H), 3.16 (s, 6H), 3.02-2.86 (m, 1H), 2.34 (s, 3H), 2.03 (br s, 1H), 1.98 (s, 3H), 1.96-1.78 (m, 1H), 1.31 (s, 2H), 1.12 (br s, 2H).

### Example 361: (S)—N-(1-(2-Methoxyquinolin-4-yl)cyclopropyl)-2-methyl-5-((1-methylazetidin-2-yl)methoxy)benzamide (Compound 555)

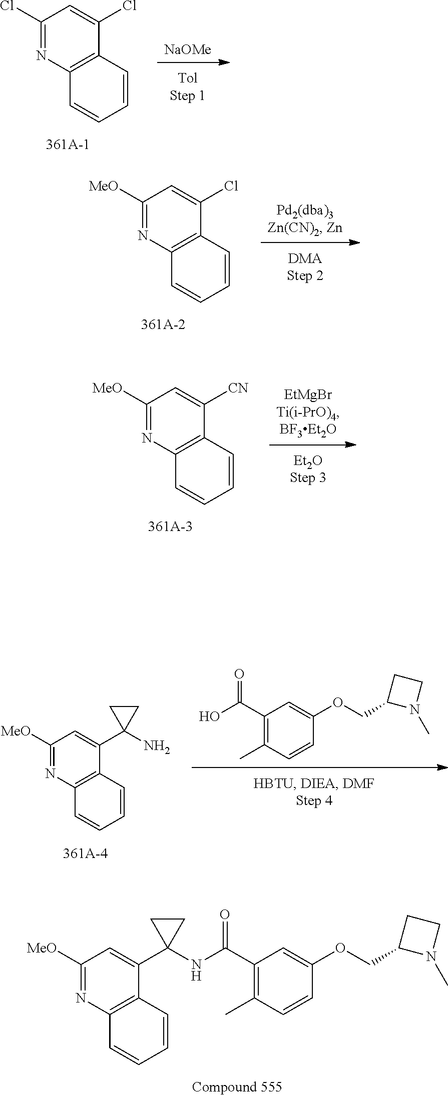

### Step 1:4-Chloro-2-methoxyquinoline (361A-2)

To a solution of 2,4-dichloroquinoline (3.00 g, 15.2 mmol, 1.0 eq) in toluene (200 mL) was added NaOMe (3.11 g, 57.6 mmol, 3.8 eq). The mixture was stirred at 120° C. for 16 h. LCMS indicated that the starting material was completely consumed, and the desired mass was detected. The reaction mixture was allowed to cool to room temperature, poured into H₂O (150 mL) and extracted with EtOAc (80 mL×3). The combined organic layers were dried over Na₂SO₄, filtered, and concentrated under vacuum to give a residue which was purified by flash silica gel chromatography using a gradient of EtOAc/petroleum ether from 1/100 to 1/10. 4-Chloro-2-methoxyquinoline (2.27 g, 11.7 mmol, 77% yield) was obtained as a white solid. M+H⁺=194.0 (LCMS).

### Step 2: 2-Methoxyquinoline-4-carbonitrile (361A-3)

To a solution of 4-chloro-2-methoxyquinoline (1.00 g, 5.16 mmol, 1.0 eq) in DMA (50 mL) were added Zn(CN)₂ (1.09 g, 9.30 mmol, 590 μL, 1.8 eq), Pd₂ (dba) 3 (709 mg, 775 μmol, 0.2 eq), Zn (101 mg, 1.55 mmol, 0.3 eq) and XPhos (492 mg, 1.03 mmol, 0.2 eq). The mixture was degassed and purged with N₂ three times. The resulting mixture was stirred at 120° C. under a N₂ atmosphere for 16 h. LCMS indicated that the starting material was completely consumed, and the desired mass was detected. The reaction mixture was allowed to cool to room temperature, poured into H₂O (50 mL) and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 1/100 to 1/10. The crude product 2-methoxyquinoline-4-carbonitrile (940 mg) was obtained as a yellow solid. M+H⁺=185.1 (LCMS).

### Step 3: 1-(2-Methoxyquinolin-4-yl)cyclopropanamine (361A-4)

A mixture of 2-methoxyquinoline-4-carbonitrile (600 mg, 3.26 mmol, 1.0 eq) in Et₂O (60 mL) was degassed and purged with N₂ three times. To this mixture was added Ti(i-PrO)₄ (1.34 g, 4.88 mmol, 1.44 mL, 1.5 eq) slowly at −78° C. and then EtMgBr (3 M, 2.38 mL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. Then BF₃·Et₂O (924 mg, 6.52 mmol, 804 μL, 2.0 eq) was added slowly with no obvious temperature change. The mixture was stirred at 20° C. for another 1 h. TLC indicated that the starting material was completely consumed. The reaction mixture was added into a mixture of HCl (1 M aqueous) (120 mL) and MTBE (30 mL), and was extracted with MTBE (30 mL×2). The aqueous layer was basified to pH 8 by using NaOH (2 M aqueous), and a precipitate was formed. The mixture was filtered through a pad of Celite and the slurry was washed with DCM several times. The combined filtrate was extracted with DCM (10 mL×4). The combined organic layers were dried over Na₂SO₄, filtered, and concentrated under vacuum to give a residue which was purified by flash silica gel chromatography using a gradient of EtOAc/petroleum ether from 1/100 to 1/0. 1-(2-Methoxyquinolin-4-yl)cyclopropanamine (190 mg, δ87 μmol, 27% yield) was obtained as a yellow oil. M+H⁺=215.1 (LCMS).

### Step 4: (S)—N-(1-(2-Methoxyquinolin-4-yl)cyclopropyl)-2-methyl-5-((1-methylazetidin-2-yl)methoxy)benzamide (Compound 555)

To a solution of 1-(2-methoxyquinolin-4-yl)cyclopropanamine (65.0 mg, 303 μmol, 1.0 eq) and(S)-2-methyl-5-((1-methylazetidin-2-yl)methoxy)benzoic acid (71.4 mg, 303 μmol, 1.0 eq) in DMF (7.0 mL) were added DIEA (118 mg, 910 μmol, 159 μL, 3.0 eq) and HBTU (127 mg, 334 μmol, 1.1 eq). The mixture was stirred at 20° C. for 16 h. LCMS indicated that the starting material was completely consumed, and the desired mass was detected. The reaction mixture was poured into H₂O (6.0 mL) and extracted with EtOAc (2.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 20%-60% B over 8 min; mobile phase A: 10 mM aqueous NH₄HCO₃, mobile phase B: acetonitrile). (S)—N-(1-(2-Methoxyquinolin-4-yl)cyclopropyl)-2-methyl-5-((1-methylazetidin-2-yl)methoxy)benzamide (4.40 mg, 10.1 μmol, 3% yield) was obtained as a white solid. M+H⁺=332.1 (LCMS); ¹H NMR (400 MHZ, DMSO-d₆) δ 9.16 (s, 1H), 8.52 (d, J=8.8 Hz, 1H), 7.80 (d, J=8.8 Hz, 1H), 7.65 (t, J=6.9 Hz, 1H), 7.46 (t, J=7.7 Hz, 1H), 7.14 (s, 1H), 7.03 (d, J=8.5 Hz, 1H), 6.84 (dd, J=2.6, 8.4 Hz, 1H), 6.62 (d, J=2.8 Hz, 1H), 3.98 (s, 3H), 3.86 (d, J=5.4 Hz, 2H), 3.24-3.11 (m, 2H), 2.73 (br d, J=2.1 Hz, 1H), 2.22 (s, 3H), 1.95 (s, 5H), 1.33 (s, 2H), 1.26-1.18 (m, 2H).

### Example 362: N-(1-(7-Fluoro-2-methoxyquinolin-4-yl)cyclopropyl)-2-methyl-5-((1-methylazetidin-2-yl)methoxy)benzamide (Compound 467)

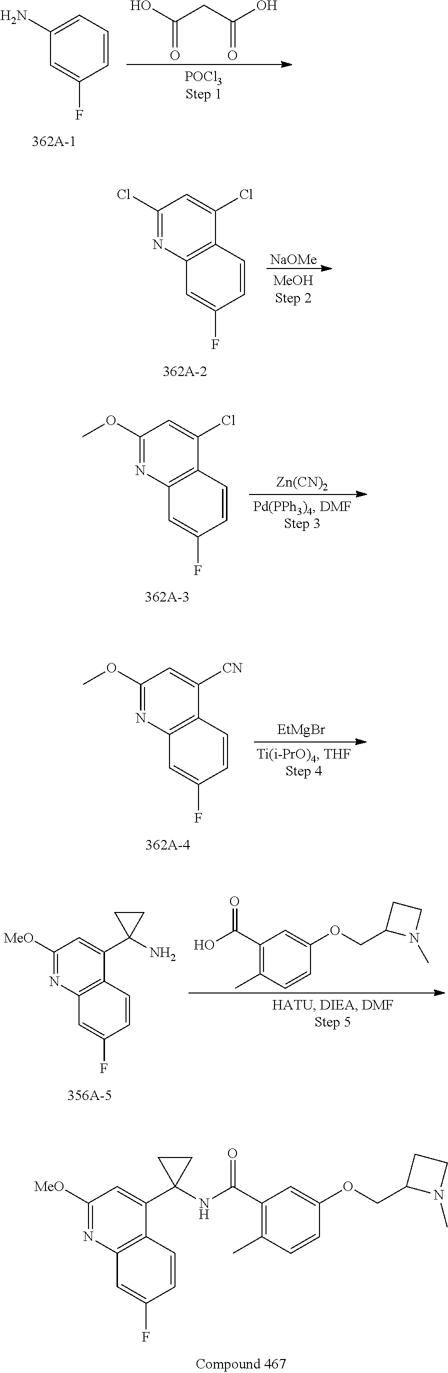

### Step 1:2,4-Dichloro-7-fluoroquinoline (362A-2)

To a solution of malonic acid (7.59 g, 72.9 mmol, 7.59 mL, 1.0 eq) in POCl₃ (30 mL) was added 3-fluoroaniline (8.10 g, 72.9 mmol, 6.98 mL, 1.0 eq) at 20° C. The mixture was stirred at 100° C. for 7 h. LCMS indicated that the starting material was completely consumed. The mixture was allowed to cool to room temperature, concentrated under vacuum to give a residue. The residue was diluted with EtOAc (20 mL), and then saturated aqueous NaHCO₃ solution was added to the solution slowly at 0° C. to adjust the pH 8. The resulting mixture was separated, and the aqueous phase was extracted with EtOAc (50 mL×3). The combined organic layers were dried over Na₂SO₄, filtered, and concentrated under vacuum to give a residue which was purified by flash silica gel chromatography using a gradient of EtOAc/petroleum ether from 0/1 to 1/100. 2,4-Dichloro-7-fluoroquinoline (5.17 g, 24.0 mmol, 33% yield) was obtained as a white solid. M+H⁺=216.2 (LCMS); ¹H NMR (400 MHZ, CDCl₃) δ 8.26-8.16 (m, 1H), 7.68 (ddd, J=9.38, 4.44, 2.31 Hz, 1H), 7.52-7.39 (m, 2H).

### Step 2:4-Chloro-7-fluoro-2-methoxyquinoline (362A-3)

To a solution of 2,4-dichloro-7-fluoroquinoline (4.77 g, 22.1 mmol, 1.0 eq) in MeOH (50 mL) was added NaOMe (19.9 g, 110 mmol, 30% purity in MeOH, 5.0 eq). The mixture was stirred at 20° C. for 3 h. LCMS indicated that the starting material was completely consumed. The reaction mixture was concentrated under vacuum to give a residue which was treated with water (50 mL) and extracted with EtOAc (50 mL×3). The combined organic layers were dried over Na₂SO₄, filtered, and concentrated under vacuum to give a residue which was purified by flash silica gel chromatography using a gradient of EtOAc/petroleum ether from 0/1 to 3/100. 4-Chloro-7-fluoro-2-methoxyquinoline (3.60 g, 15.7 mmol, 71% yield) was obtained as a white solid. M+H⁺=212.1 (LCMS).

### Step 3: 7-Fluoro-2-methoxyquinoline-4-carbonitrile (362A-4)

To a solution of 4-chloro-7-fluoro-2-methoxyquinoline (1.40 g, 6.62 mmol, 1.0 eq) in DMF (30 mL) were added Zn(CN)₂ (1.55 g, 13.2 mmol, 2.0 eq) and Pd(PPh₃)₄ (764 mg, 662 μmol, 0.1 eq). The mixture was stirred at 120° C. for 16 h under a N₂ atmosphere. LCMS indicated that the starting material was completely consumed. The reaction mixture was allowed to cool to room temperature, poured into H₂O (30 mL) and extracted with EtOAc (30 mL×3). The combined organic layers were washed with brine (90 mL×3), dried over Na₂SO₄, filtered, and concentrated under vacuum to give a residue which was purified by flash silica gel chromatography using a gradient of EtOAc/petroleum ether from 0/1 to 1/10. 7-Fluoro-2-methoxyquinoline-4-carbonitrile (1.14 g, 4.86 mmol, 73% yield) was obtained as a white solid. M+H⁺=203.1 (LCMS).

### Step 4: 1-(7-Fluoro-2-methoxyquinolin-4-yl)cyclopropanamine (362A-5)

To a solution of 7-fluoro-2-methoxyquinoline-4-carbonitrile (100 mg, 495 μmol, 1.0 eq) in anhydrous Et₂O (40 mL) was degassed and purged with N₂ three times. The mixture was stirred at −78° C. To this mixture was added Ti(i-PrO)₄ (211 mg, 742 μmol, 219 μL, 1.5 eq) slowly, and then EtMgBr (3 M in Et₂O, 363 μL, 2.2 eq) was added dropwise to maintain the temperature between −78° C. and −75° C. over 10 min under a N₂ atmosphere. After the addition was complete, the resulting mixture was stirred at the same temperature for 10 min and warmed to 20° C. over 1 h. BF₃·Et₂O (140 mg, 989 μmol, 122 μL, 2.0 eq) was added slowly with no obvious temperature change. The mixture was stirred at 20° C. for another 1 h. LCMS indicated that the starting material was completely consumed. The reaction mixture was added into a mixture of HCl (1 M aqueous) (40 mL) and MTBE (40 mL) and extracted with MTBE (40 mL×2). The organic phase was discarded. The aqueous layer was basified to pH 8 by using NaOH (2 M aqueous), and a precipitate was formed. The mixture was filtered through a pad of Celite and the slurry was washed with DCM several times. The combined filtrate was extracted with DCM (50 mL×3). The combined organic layers were dried over Na₂SO₄, filtered, and concentrated under vacuum to give a residue which was purified by flash silica gel chromatography using a gradient of EtOAc/petroleum ether from 0/1 to 1/0. 1-(7-Fluoro-2-methoxyquinolin-4-yl)cyclopropanamine (150 mg, 520 μmol, 35% yield) was obtained as a yellow oil. M+H⁺=233.3 (LCMS).

### Step 5: N-(1-(7-Fluoro-2-methoxyquinolin-4-yl)cyclopropyl)-2-methyl-5-((1-methylazetidin-2-yl)methoxy)benzamide (Compound 467)

To a solution of 1-(7-fluoro-2-methoxyquinolin-4-yl)cyclopropanamine (120 mg, 517 μmol, 1.5 eq) and 2-methyl-5-((1-methylazetidin-2-yl)methoxy)benzoic acid (93.6 mg, 344 μmol, 1.0 eq, HCl salt) in DMF (2.0 mL) were added HBTU (327 mg, 861 μmol, 2.5 eq) and DIEA (223 mg, 1.72 mmol, 300 μL, 5.0 eq). The mixture was stirred at 20° C. for 16 h. LCMS indicated that the starting material was completely consumed. The mixture was treated with H₂O (3.0 mL) and extracted with EtOAc (3.0 mL×3). The combined organic layers were washed with brine (9.0 mL×3), dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 40%-75% B over 8 min; mobile phase A: 10 mM aqueous NH₄HCO₃ with NH₃·H₂O (0.05%), mobile phase B: acetonitrile). N-(1-(7-Fluoro-2-methoxyquinolin-4-yl)cyclopropyl)-2-methyl-5-((1-methyl azetidin-2-yl)methoxy)benzamide (14.7 mg, 32.7 μmol, 10% yield) was obtained as a pale yellow solid. M+H⁺=450.3 (LCMS); ¹H NMR (400 MHZ, CD₃OD) δ 8.51-8.45 (m, 1H), 7.54-7.46 (m, 1H), 7.29-7.23 (m, 1H), 7.23-7.20 (m, 1H), 7.05 (d, J=8.4 Hz, 1H), 6.87-6.83 (m, 1H), 6.65 (d, J=2.6 Hz, 1H), 4.06-4.02 (m, 3H), 3.91 (d, J=5.4 Hz, 2H), 3.51-3.43 (m, 1H), 3.42-3.36 (m, 1H), 2.98-2.89 (m, 1H), 2.39-2.35 (m, 3H), 2.10-2.01 (m, 2H), 2.01-1.98 (m, 3H), 1.46-1.40 (m, 2H), 1.34-1.29 (m, 2H).

### Example 363: 5-(Azetidin-3-ylamino)-2-methyl-N-(1-(3-(5-(pyrrolidin-1-ylmethyl)thiophen-2-yl)phenyl)cyclopropyl)benzamide (Compound 565)

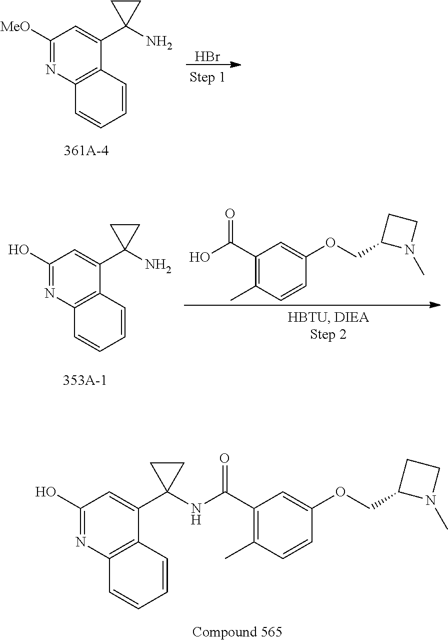 [SMILES: COc1cc(C2(N)CC2)c2ccccc2n1 ; Br ; NC1(c2cc(O)nc3ccccc23)CC1 ; Cc1ccc(OC[C@@H]2CCN2C)cc1C(=O)O ; Cc1ccc(OC[C@@H]2CCN2C)cc1C(=O)NC1(c2cc(O)nc3ccccc23)CC1]

### Step 1:4-(1-Aminocyclopropyl)quinolin-2-ol (363A-1)

A solution of 1-(2-methoxyquinolin-4-yl)cyclopropanamine (180 mg, 840 μmol, 1.0 eq) in HBr (30 mL, 30% purity in water) was stirred at 130° C. for 16 h. LCMS indicated that the starting material was completely consumed, and the desired mass was detected. The reaction mixture was allowed to cool to room temperature, poured into NaHCO₃ aqueous (30 mL) at 25° C. and extracted with EtOAc (10 mL×3). The combined organic layers were dried over Na₂SO₄, filtered, and concentrated under vacuum to give a residue was used in the next step without any further purification. 4-(1-Aminocyclopropyl)quinolin-2-ol (120 mg, 600 μmol, 72% yield) was obtained as a yellow solid. M+H⁺=201.1 (LCMS).

### Step 2: (S)—N-(1-(2-Hydroxyquinolin-4-yl)cyclopropyl)-2-methyl-5-((1-methylazetidin-2-yl)methoxy)benzamide (Compound 565)

To a solution of 4-(1-aminocyclopropyl)quinolin-2-ol (110 mg, 549 μmol, 1.0 eq) and(S)-2-methyl-5-((1-methylazetidin-2-yl)methoxy)benzoic acid (129 mg, 549 μmol, 1.0 eq) in DMF (6.0 mL) were added DIEA (213 mg, 1.65 mmol, 287 μL, 3.0 eq) and HBTU (229 mg, 604 μmol, 1.1 eq). The mixture was stirred at 20° C. for 16 h. LCMS indicated that the starting material was completely consumed, and the desired mass was detected. The reaction mixture was poured into H₂O (15 mL) and extracted with EtOAc (6.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 1%-30% B over 8 min; mobile phase A: 0.04% aqueous HCl, mobile phase B: acetonitrile). (S)—N-(1-(2-hydroxyquinolin-4-yl)cyclopropyl)-2-methyl-5-((1-methylazetidin-2-yl)methoxy)benzamide (7.8 mg, 16.8 μmol, 3% yield, HCl salt) was obtained as a white solid. M+H⁺=418.1 (LCMS); ¹H NMR (400 MHZ, DMSO-d₆) δ 9.10 (s, 1H), 8.27 (br d, J=8.4 Hz, 1H), 7.50 (br t, J=7.6 Hz, 1H), 7.34 (br d, J=8.3 Hz, 1H), 7.21 (br t, J=7.4 Hz, 1H), 7.11 (br d, J=8.5 Hz, 1H), 6.93 (br dd, J=2.5, 8.5 Hz, 1H), 6.74 (br d, J=1.9 Hz, 1H), 6.68 (s, 1H), 4.69-4.57 (m, 1H), 4.36-4.19 (m, 2H), 4.11-3.97 (m, 1H), 3.93-3.79 (m, 1H), 2.90-2.79 (m, 3H), 2.39-2.26 (m, 2H), 1.99 (s, 3H), 1.27 (br s, 2H), 1.18 (br s, 2H).

### Example 364: (S)—N-(1-(7-Fluoro-2-hydroxyquinolin-4-yl)cyclopropyl)-2-methyl-5-((1-methylazetidin-2-yl)methoxy)benzamide (Compound 542)

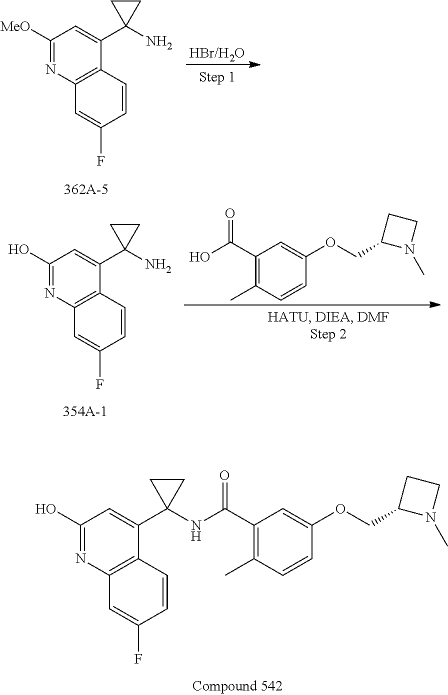 [SMILES: COc1cc(C2(N)CC2)c2ccc(F)cc2n1 ; "HBr/H2O" ; NC1(c2cc(O)nc3cc(F)ccc23)CC1 ; Cc1ccc(OC[C@@H]2CCN2C)cc1C(=O)O ; Cc1ccc(OC[C@@H]2CCN2C)cc1C(=O)NC1(c2cc(O)nc3cc(F)ccc23)CC1]

### Step 1:4-(1-Aminocyclopropyl)-7-fluoroquinolin-2-ol (364A-1)

To a solution of HBr (30 mL, 30% purity in water) was added 1-(7-fluoro-2-methoxyquinolin-4-yl)cyclopropanamine (320 mg, 1.38 mmol, 1.0 eq). The mixture was stirred at 120° C. for 2 h. LCMS indicated that the starting material was completely consumed, and the desired mass was detected. The mixture was allowed to cool to room temperature and concentrated under vacuum at 30° C. to give a residue which was purified by preparative HPLC (Phenomenex Luna C18 column (75×30 mm, 3 μm); flow rate: 25 mL/min; gradient: 10%-40% B over 8 min; mobile phase A: 0.1% aqueous TFA, mobile phase B: acetonitrile). 4-(1-Aminocyclopropyl)-7-fluoroquinolin-2-ol (150 mg, 513 μmol, 37% yield, TFA salt) was obtained as a yellow oil. M+H⁺=219.2 (LCMS).

### Step 2: (S)—N-(1-(7-Fluoro-2-hydroxyquinolin-4-yl)cyclopropyl)-2-methyl-5-((1-methylazetidin-2-yl)methoxy)benzamide (Compound 542)

To a solution of 4-(1-aminocyclopropyl)-7-fluoroquinolin-2-ol (120 mg, 550 μmol, 1.0 eq, TFA salt) in DMF (1.0 mL) was added(S)-2-methyl-5-((1-methylazetidin-2-yl)methoxy)benzoic acid (129 mg, 550 μmol, 1.2 eq), followed by HBTU (521 mg, 1.37 mmol, 2.5 eq) and DIEA (210 mg, 1.65 mmol, 2.87 mL, 3.0 eq). The mixture was stirred at 20° C. for 16 h. LCMS indicated that the starting material was completely consumed, and the desired mass was detected. The mixture was concentrated under vacuum at 30° C. to give a residue which was purified by preparative HPLC (Phenomenex Luna column (80×30 mm, 3 μm); flow rate: 25 mL/min; gradient: 10%-35% B over 8 min; mobile phase A: 0.04% aqueous HCl, mobile phase B: acetonitrile) to give(S)—N-(1-(7-fluoro-2-hydroxyquinolin-4-yl)cyclopropyl)-2-methyl-5-((1-methylazetidin-2-yl)methoxy)benzamide (32.0 mg, 69.8 μmol, 13% yield, HCl salt) as a white solid. M+H⁺=436.0 (LCMS); ¹H NMR (400 MHZ, DMSO-d₆) δ ppm 11.79 (br s, 1H), 9.07 (s, 1H), 8.37-8.26 (m, 1H), 7.13-7.03 (m, 3H), 6.85 (dd, J=8.44, 2.52 Hz, 1H), 6.65-6.57 (m, 2H), 3.87 (d, J=5.48 Hz, 2H), 3.26-3.19 (m, 2H), 2.75-2.67 (m, 1H), 2.22 (s, 3H), 2.00 (s, 3H), 1.98-1.79 (m, 2H), 1.29-1.23 (m, 2H), 1.19-1.13 (m, 2H).

### Example 365: (S)—N-(1-(2-Chloro-7-fluoroquinolin-4-yl)cyclopropyl)-2-methyl-5-((1-methylazetidin-2-yl)methoxy)benzamide (Compound 586)

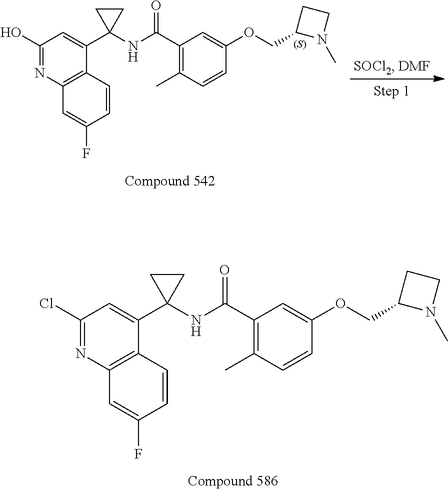

### Step 1: (S)—N-(1-(2-Chloro-7-fluoroquinolin-4-yl)cyclopropyl)-2-methyl-5-((1-methylazetidin-2-yl)methoxy)benzamide (Compound 586)

To a solution of(S)—N-(1-(7-fluoro-2-hydroxyquinolin-4-yl)cyclopropyl)-2-methyl-5-((1-methylazetidin-2-yl)methoxy)benzamide (43.0 mg, 98.7 μmol, 1.0 eq) in SOCl₂ (1.7 mL) was added DMF (100 μL). The mixture was stirred at 20° C. for 3.5 h. LCMS indicated that the starting material was completely consumed. The reaction mixture was quenched by saturated aqueous NaHCO₃ (30 mL) at 0° C. and extracted with EtOAc (15 mL×3). The combined organic layers were dried over Na₂SO₄, filtered, and concentrated under vacuum to give a residue which was purified by preparative HPLC (Phenomenex Gemini C18 column (800× 30 mm, 3 μm); flow rate: 60 mL/min; gradient: 10%-40% B over 8 min; mobile phase A: 0.04% aqueous HCl, mobile phase B: acetonitrile). (S)—N-(1-(2-Chloro-7-fluoroquinolin-4-yl)cyclopropyl)-2-methyl-5-((1-methylazetidin-2-yl)methoxy)benzamide (2.30 mg, 4.69 μmol, 5% yield) was obtained as a yellow gum. M+H⁺=454.1 (LCMS); ¹H NMR (400 MHZ, DMSO-d₆) δ 9.35 (s, 1H), 8.74 (dd, J=6.2, 9.3 Hz, 1H), 7.79 (dd, J=2.6, 10.1 Hz, 1H), 7.72-7.63 (m, 2H), 7.15-7.06 (m, 1H), 6.93 (dd, J=2.6, 8.3 Hz, 1H), 6.81-6.68 (m, 1H), 4.70-4.58 (m, 1H), 4.37-4.28 (m, 1H), 4.27-4.18 (m, 1H), 4.05-3.97 (m, 1H), 3.91-3.85 (m, 1H), 2.86-2.80 (m, 3H), 2.40-2.27 (m, 2H), 2.00-1.90 (m, 3H), 1.41-1.31 (m, 4H).

### Example 366: (S)—N-(1-(7-Methoxyisoquinolin-5-yl)cyclopropyl)-2-methyl-5-((1-methylazetidin-2-yl)methoxy)benzamide (Compound 538)

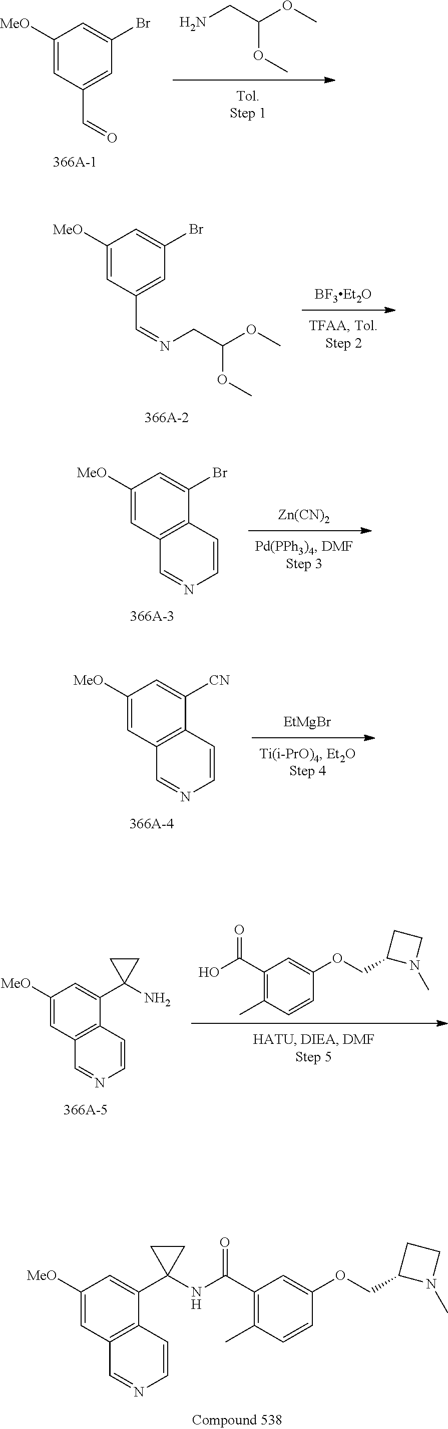

### Step 1: (Z)—N-(3-Bromo-5-methoxybenzylidene)-2,2-dimethoxyethanamine (366A-2)

To a solution of 3-bromo-5-methoxybenzaldehyde (21.0 g, 97.7 mmol, 1.0 eq) in toluene (220 mL) was added 2,2-dimethoxyethanamine (10.3 g, 97.7 mmol, 10.6 mL, 1.0 eq) at 20° C. The mixture was stirred at 110° C. for 16 h. LCMS indicated that the starting material was completely consumed. The reaction mixture was allowed to cool to room temperature, concentrated under vacuum to give a crude product (Z)—N-(3-bromo-5-methoxybenzylidene)-2,2-dimethoxyethanamine (30.0 g) as a yellow oil. M+H⁺=302.0 (LCMS).

### Step 2: 5-Bromo-7-methoxyisoquinoline (366A-3)

To a solution of (Z)—N-(3-bromo-5-methoxybenzylidene)-2,2-dimethoxyethanamine (10.0 g, 33.0 mmol, 1.0 eq) in toluene (30 mL) were added TFAA (13.9 g, 66.2 mmol, 9.21 mL, 2.0 eq) and BF₃·Et₂O (9.39 g, 66.2 mmol, 8.17 mL, 2.0 eq) at 0° C. The mixture was stirred at 20° C. for 16 h. LCMS indicated that the starting material was completely consumed. The reaction mixture was concentrated under vacuum to give a residue which was treated with water (50 mL), the aqueous layer was adjusted to pH 8 by using saturated aqueous NaHCO₃. The aqueous layer was then extracted with EtOAc (50 mL×3). The combined organic layers were dried over Na₂SO₄, filtered, and concentrated under vacuum to give a residue which was purified by flash silica gel chromatography using a gradient of EtOAc/petroleum ether from 0/1 to 1/9. 5-Bromo-7-methoxyisoquinoline (8.30 g, 34.8 mmol, 35% yield) was obtained as a yellow solid. M+H⁺=238.1 (LCMS).

### Step 3: 7-Methoxyisoquinoline-5-carbonitrile (366A-4)

To a solution of 5-bromo-7-methoxyisoquinoline (300 mg, 1.26 mmol, 1.0 eq) in DMF (4.0 mL) were added Zn(CN)₂ (370 mg, 3.15 mmol, 2.5 eq) and Pd(PPh₃)₄ (146 mg, 126 μmol, 0.1 eq). The mixture was stirred at 120° C. for 16 h under a N₂ atmosphere. LCMS indicated that the starting material was completely consumed. The reaction mixture was allowed to cool to room temperature, poured into H₂O (10 mL) and extracted with EtOAc (10 mL×3). The combined organic layers were washed with brine (30 mL×3), dried over Na₂SO₄, filtered, and concentrated under vacuum to give a residue which was purified by flash silica gel chromatography using a gradient of EtOAc/petroleum ether from 0/1 to 1/2. 7-Methoxyisoquinoline-5-carbonitrile (210 mg, 842 μmol, 67% yield) was obtained as a yellow solid. M+H⁺=185.3 (LCMS).

### Step 4: 1-(7-Methoxyisoquinolin-5-yl)cyclopropanamine (366A-5)

A mixture of 7-methoxyisoquinoline-5-carbonitrile (50.0 mg, 271 μmol, 1.0 eq) in anhydrous Et₂O (10 mL) was degassed and purged with N₂ three times. The mixture was stirred at −78° C. To this mixture was added Ti(i-PrO)₄ (116 mg, 407 μmol, 120 μL, 1.5 eq) slowly, and then EtMgBr (3 M in Et₂O, 199 μL, 2.2 eq) was added dropwise to maintain the temperature between −78° C. and −75° C. under a N₂ atmosphere. After the addition was complete, the resulting mixture was stirred at the same temperature for 10 min and warmed to 20° C. over 1 h. BF₃·Et₂O (77.1 mg, 543 μmol, 67.0 μL, 2.0 eq) was added slowly with no obvious temperature change. The mixture was stirred at 20° C. for another 1 h. LCMS indicated that 29% starting material remained and 50% desired compound was detected. The reaction mixture was added into a mixture of HCl (1 M aqueous) (60 mL) and MTBE (60 mL) and extracted with MTBE (60 mL×2). The organic phase was discarded. The aqueous layer was basified to pH 8 by using NaOH (2 M aqueous), and a precipitate was formed. The mixture was filtered through a pad of Celite and the slurry was washed with DCM several times. The combined filtrate was extracted with DCM (60 mL×3). The combined organic layers were dried over Na₂SO₄, filtered, and concentrated under vacuum to give a residue which was purified by flash silica gel chromatography using a gradient of EtOAc/petroleum ether from 0/1 to 100/1. 1-(7-Methoxyisoquinolin-5-yl)cyclopropanamine (12.5 mg, 30.5 μmol, 11% yield) was obtained as a yellow oil. M+H⁺=215.3 (LCMS).

### Step 5: (S)—N-(1-(7-Methoxyisoquinolin-5-yl)cyclopropyl)-2-methyl-5-((1-methylazetidin-2-yl)methoxy)benzamide (Compound 538)

To a solution of 1-(7-methoxyisoquinolin-5-yl)cyclopropanamine (50.0 mg, 233 μmol, 1.0 eq) and(S)-2-methyl-5-((1-methylazetidin-2-yl)methoxy)benzoic acid (54.9 mg, 233 μmol, 1.0 eq) in DMF (1.0 mL) were added HATU (177 mg, 467 μmol, 2.0 eq) and DIEA (90.5 mg, 700 μmol, 122 μL, 3.0 eq). The resulting mixture was stirred at 20° C. for 16 h. LCMS indicated that the starting material was completely consumed. The reaction mixture was concentrated under vacuum to give a residue which was purified by preparative HPLC (Phenomenex C18 column (75×30 mm, 3 μm); flow rate: 25 mL/min; gradient: 20%-45% B over 8 min; mobile phase A: 10 mM aqueous NH₄HCO₃ with NH₃·H₂O (0.05%), mobile phase B: acetonitrile). (S)—N-(1-(7-Methoxyisoquinolin-5-yl)cyclopropyl)-2-methyl-5-((1-methylazetidin-2-yl)methoxy)benzamide (11.3 mg, 25.3 μmol, 11% yield) was obtained as a white solid. M+H⁺=432.1 (LCMS); ¹H NMR (400 MHZ, DMSO-d₆) δ 9.25-9.17 (m, 1H), 9.17-9.09 (m, 1H), 8.48-8.39 (m, 1H), 8.39-8.33 (m, 1H), 7.61 (d, J=2.5 Hz, 1H), 7.43 (d, J=2.4 Hz, 1H), 7.03 (d, J=8.4 Hz, 1H), 6.89-6.81 (m, 1H), 6.61 (d, J=2.8 Hz, 1H), 3.95-3.89 (m, 3H), 3.86 (d, J=5.4 Hz, 2H), 3.28-3.20 (m, 2H), 2.75-2.67 (m, 1H), 2.27-2.19 (m, 3H), 2.04-1.90 (m, 4H), 1.90-1.79 (m, 1H), 1.39-1.24 (m, 2H), 1.23-1.15 (m, 2H).

### Example 367: N-(1-(3-Hydroxyisoquinolin-5-yl)cyclopropyl)-2-methyl-5-((1-methylazetidin-2-yl)methoxy)benzamide (Compound 509)

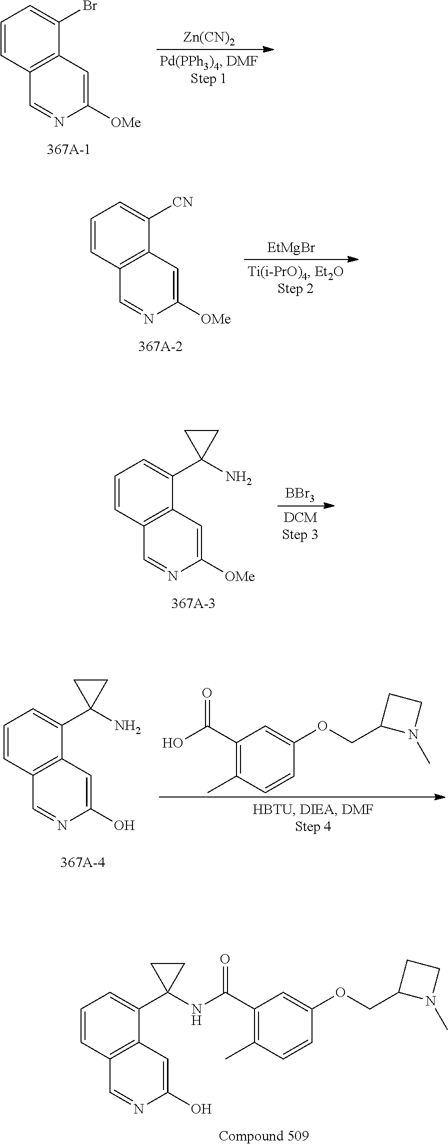

### Step 1:3-Methoxyisoquinoline-5-carbonitrile (367A-2)

To a solution of 5-bromo-3-methoxyisoquinoline (4.00 g, 16.8 mmol, 1.0 eq) in DMF (40 mL) were added Zn(CN)₂ (4.10 g, 35.0 mmol, 2.22 mL, 2.1 eq) and Pd(PPh₃)₄ (1.94 g, 1.68 mmol, 0.1 eq). The mixture was stirred at 120° C. for 16 h under a N₂ atmosphere. LCMS indicated that the starting material was completely consumed. The reaction mixture was allowed to cool to room temperature, poured into H₂O (40 mL) and extracted with EtOAc (40 mL×3). The combined organic layers were washed with brine (120 mL×3), dried over Na₂SO₄, filtered, and concentrated under vacuum to give a residue which was purified by flash silica gel chromatography using a gradient of EtOAc/petroleum ether from 0/1 to 4/10. 3-Methoxyisoquinoline-5-carbonitrile (3.00 g, 16.2 mmol, 96% yield) was obtained as a white solid. M+H⁺=185.1 (LCMS).

### Step 2: 1-(3-Methoxyisoquinolin-5-yl)cyclopropanamine (367A-3)

A mixture of 3-methoxyisoquinoline-5-carbonitrile (500 mg, 2.71 mmol, 1.0 eq) in anhydrous Et₂O (100 mL) was degassed and purged with N₂ three times. The mixture was stirred at −78° C. To this mixture was added Ti(i-PrO)₄ (1.16 g, 4.07 mmol, 1.20 mL, 1.5 eq) slowly, and then EtMgBr (3 M in Et₂O, 1.99 mL, 2.2 eq) was added dropwise to maintain the temperature between −78° C. and −75° C. under a N₂ atmosphere. After the addition was completed, the resulting mixture was stirred at the same temperature for 10 min and warmed to 20° C. over 1 h. BF₃·Et₂O (771 mg, 5.43 mmol, 670 μL, 2.0 eq) was added slowly with no obvious temperature change. The mixture was stirred at 20° C. for another 1 h. LCMS indicated that 12% starting material remained and 40% desired compound was detected. The reaction mixture was added into a mixture of HCl (1 M aqueous) (100 mL) and MTBE (100 mL) and extracted with MTBE (100 mL×2). The organic phase was discarded. The aqueous layer was basified to pH 8 by using NaOH (2 M aqueous), and a precipitate was formed. The mixture was filtered through a pad of Celite and the slurry was washed with DCM several times. The combined filtrate was extracted with DCM (100 mL×3). The combined organic layers were dried over Na₂SO₄, filtered, and concentrated under vacuum to give a residue which was purified by flash silica gel chromatography using a gradient of (DCM/MeOH=10:1)/petroleum ether from 0/1 to 2/10. 1-(3-Methoxyisoquinolin-5-yl)cyclopropanamine (300 mg, 1.29 mmol, 24% yield) was obtained as a yellow oil. M+H⁺=215.2 (LCMS).

### Step 3: 5-(1-Aminocyclopropyl)isoquinolin-3-ol (367A-4)

To a solution of 1-(3-methoxyisoquinolin-5-yl)cyclopropanamine (200 mg, 933 μmol, 1.0 eq) in DCM (2.0 mL) was added BBr₃ (3.51 g, 14.0 mmol, 1.35 mL, 15 eq) dropwise at −78° C. The mixture was stirred at 20° C. for 16 h under a N₂ atmosphere. LCMS and HPLC indicated that the starting material was completely consumed. The reaction mixture was poured into saturated aqueous NaHCO₃ solution (10 mL) slowly at 0° C., extracted with DCM (2.0 mL×3). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 1%-25% B over 8 min; mobile phase A: 0.1% aqueous TFA, mobile phase B: acetonitrile) to give 5-(1-aminocyclopropyl)isoquinolin-3-ol (40.0 mg, 127 μmol, 14% yield, TFA salt) as a red solid. M+H⁺=201.3 (LCMS).

### Step 4: N-(1-(3-Hydroxyisoquinolin-5-yl)cyclopropyl)-2-methyl-5-((1-methylazetidin-2-yl)methoxy)benzamide (Compound 509)

To a solution of 5-(1-aminocyclopropyl)isoquinolin-3-ol (35.0 mg, 111 μmol, 1.0 eq, TFA salt) and 2-methyl-5-((1-methylazetidin-2-yl)methoxy)benzoic acid (26.2 mg, 96.4 μmol, 0.9 eq, HCl salt) in DMF (1.0 mL) were added HBTU (84.5 mg, 223 μmol, 2.0 eq) and DIEA (43.2 mg, 334 μmol, 58.2 μL, 3.0 eq). The resulting mixture was stirred at 20° C. for 16 h. LCMS indicated that the starting material was completely consumed. The reaction mixture was concentrated under vacuum to give a residue which was purified by preparative HPLC (Phenomenex Luna C18 column (80×30 mm, 3 μm); flow rate: 25 mL/min; gradient: 5%-40% B over 8 min; mobile phase A: 0.1% aqueous TFA, mobile phase B: acetonitrile) to give N-(1-(3-hydroxyisoquinolin-5-yl)cyclopropyl)-2-methyl-5-((1-methylazetidin-2-yl)methoxy)benzamide (10.7 mg, 18.8 μmol, 17% yield, TFA salt) as a yellow solid. M+H⁺=418.1 (LCMS); ¹H NMR (400 MHZ, CD₃OD) δ 8.83-8.65 (m, 1H), 8.02-7.92 (m, 1H), 7.80 (d, J=8.5 Hz, 1H), 7.57-7.50 (m, 1H), 7.31-7.19 (m, 1H), 7.13 (d, J=8.4 Hz, 1H), 6.98-6.93 (m, 1H), 6.79 (d, J=2.8 Hz, 1H), 4.74-4.63 (m, 1H), 4.33-4.26 (m, 1H), 4.26-4.09 (m, 2H), 4.02-3.90 (m, 1H), 3.02-2.90 (m, 3H), 2.61-2.50 (m, 2H), 2.12-2.05 (m, 3H), 1.45-1.38 (m, 2H), 1.30-1.24 (m, 2H).

### Example 368: N-(3-Fluorobenzyl)-1-(1-(quinolin-5-yl)ethyl)piperidine-4-carboxamide (Compound 549)

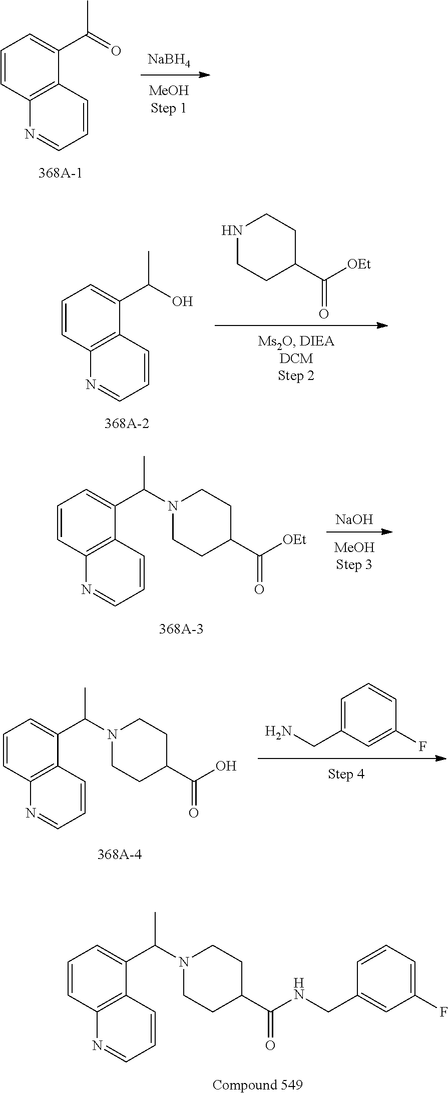

### Step 1: 1-(Quinolin-5-yl)ethanol (368A-2)

To a solution of 1-(quinolin-5-yl)ethanone (200 mg, 1.17 mmol, 1.0 eq) in MeOH (12 mL) was added NaBH₄ (66.3 mg, 1.75 mmol, 1.5 eq) at 0° C. The mixture was stirred at 20° C. for 30 min. LCMS indicated that the starting material was completely consumed, and the desired mass was detected. The mixture was poured into ice water (15 mL) and extracted with DCM (10 mL×3). The combined organic layers were dried over Na₂SO₄, filtered, and concentrated under vacuum to give a residue which was purified by preparative TLC (DCM/MeOH=10/1, R_{f}=0.6). 1-(Quinolin-5-yl)ethanol (120 mg, 693 μmol, 60% yield) was obtained as a colorless oil. M+H⁺=174.2 (LCMS); ¹H NMR (400 MHZ, CDCl₃) δ 8.94 (dd, J=1.5, 4.1 Hz, 1H), 8.58 (d, J=8.8 Hz, 1H), 8.11-8.03 (m, 1H), 7.77-7.69 (m, 2H), 7.45 (dd, J=4.1, 8.6 Hz, 1H), 5.72-5.53 (m, 1H), 2.06-1.99 (m, 1H), 1.70 (d, J=6.5 Hz, 3H).

### Step 2: Ethyl 1-(1-(quinolin-5-yl)ethyl)piperidine-4-carboxylate (368A-3)

To a solution of 1-(quinolin-5-yl)ethanol (70.0 mg, 404 μmol, 1.0 eq) and DIEA (209 mg, 1.62 mmol, 282 μL, 4.0 eq) in DCM (5.0 mL) was added a mixture of Ms₂O (70.4 mg, 404 μmol, 1.0 eq) in DCM (500 μL) dropwise at 0° C. The mixture was stirred at 0° C. for 30 min. Then ethyl piperidine-4-carboxylate (63.5 mg, 404 μmol, 62.3 μL, 1.0 eq) was added. The mixture was stirred at 20° C. for 14 h. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give a residue which was purified by preparative TLC (DCM/MeOH=10/1, R_{f}=0.5). Ethyl 1-(1-(quinolin-5-yl)ethyl)piperidine-4-carboxylate (100 mg) was obtained as a colorless oil.

### Step 3: 1-(1-(Quinolin-5-yl)ethyl)piperidine-4-carboxylic acid (368A-4)

To a solution of ethyl 1-(1-(quinolin-5-yl)ethyl)piperidine-4-carboxylate (100 mg, 320 μmol, 1.0 eq) in a mixture of MeOH (3.0 mL) and H₂O (0.6 mL) was added NaOH (1 M aqueous, 640 μL, 2.0 eq). The mixture was stirred at 20° C. for 16 h. LCMS indicated that the starting material was completely consumed, and the desired mass was detected. H₂O (5.0 mL) was added and the mixture was washed with MTBE (3.0 mL×2). The aqueous layer was acidified to pH 5 with HCl (1 M aqueous). The product was extracted with EtOAc (3.0 mL×3). The combined organic layers were dried over Na₂SO₄, filtered, and concentrated under vacuum to give the crude product 1-(1-(quinolin-5-yl)ethyl)piperidine-4-carboxylic acid (100 mg), which was used in the next step without any further purification. M+H⁺=285.0 (LCMS).

### Step 4: N-(3-Fluorobenzyl)-1-(1-(quinolin-5-yl)ethyl)piperidine-4-carboxamide (Compound 549)

To a solution of 1-(1-(quinolin-5-yl)ethyl)piperidine-4-carboxylic acid (100 mg, 352 μmol, 1.0 eq) and (3-fluorophenyl)methanamine (48.4 mg, 387 μmol, 44.0 μL 1.1 eq) in DMF (5.0 mL) were added DIEA (45.5 mg, 352 μmol, 61.3 μL, 1.0 eq), EDCI (67.4 mg, 352 μmol, 1.0 eq) and HOBt (57.0 mg, 422 μmol, 1.2 eq). The mixture was stirred at 20° C. for 14 h. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give a residue which was purified by preparative HPLC (Phenomenex Gemini C18 column (80×30 mm, 3 μm); flow rate: 25 mL/min; gradient: 1%-30% B over 8 min; mobile phase A: 0.04% aqueous HCl, mobile phase B: acetonitrile). N-(3-Fluorobenzyl)-1-(1-(quinolin-5-yl)ethyl)piperidine-4-carboxamide (19.3 mg, 45.1 μmol, 13% yield, HCl salt) was obtained as a white solid. M+H⁺=392.1 (LCMS); ¹H NMR (400 MHZ, DMSO-d₆) δ 10.76-10.57 (m, 1H), 9.23-9.10 (m, 2H), 8.57-8.46 (m, 1H), 8.38 (br d, J=7.1 Hz, 1H), 8.26 (br d, J=8.4 Hz, 1H), 8.04 (br t, J=7.9 Hz, 1H), 7.85 (br dd, J=4.2, 8.4 Hz, 1H), 7.45-7.26 (m, 1H), 7.16-6.93 (m, 3H), 5.55-5.34 (m, 1H), 4.26 (br d, J=5.9 Hz, 2H), 3.95 (br d, J=12.1 Hz, 1H), 3.20-2.77 (m, 3H), 2.41 (br d, J=4.0 Hz, 1H), 2.20-1.97 (m, 2H), 1.91-1.75 (m, 5H).

### Example 369: N-(3-Fluorobenzyl)-1-(1-(isoquinolin-5-yl)ethyl)piperidine-4-carboxamide (Compound 522)

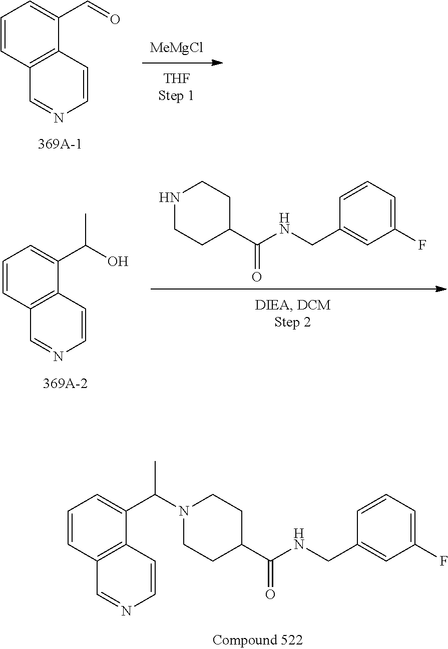 [SMILES: "MeMgCl" ; O=Cc1cccc2cnccc12 ; CC(O)c1cccc2cnccc12 ; O=C(NCc1cccc(F)c1)C1CCNCC1 ; CC(c1cccc2cnccc12)N1CCC(C(=O)NCc2cccc(F)c2)CC1]

### Step 1: 1-(Isoquinolin-5-yl)ethanol (369A-2)

A solution of isoquinoline-5-carbaldehyde (500 mg, 3.18 mmol, 1.0 eq) in THF (10 mL) was degassed and purged with N₂ three times. To this mixture was added MeMgBr (3 M in Et₂O, 1.06 mL, 1.0 eq) dropwise at −78° C. The mixture was stirred at the same temperature for 30 min under a N₂ atmosphere. LCMS indicated that the starting material was completely consumed, and the desired mass was detected. The reaction mixture was poured into H₂O (15 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/3. 1-(Isoquinolin-5-yl)ethanol (238 mg, 1.37 mmol, 43% yield) was obtained as a yellow solid. M+H⁺=174.1 (LCMS).

### Step 2: N-(3-Fluorobenzyl)-1-(1-(isoquinolin-5-yl)ethyl)piperidine-4-carboxamide (Compound 522)

To a solution of 1-(isoquinolin-5-yl)ethanol (127 mg, 733 μmol, 1.0 eq) in DCM (5.0 mL) were added DIEA (379 mg, 2.93 mmol, 511 μL, 4.0 eq) and methylsulfonyl methanesulfonate (128 mg, 733 μmol, 1.0 eq) at 0° C. The mixture was stirred at 0° C. for 30 min. N-(3-Fluorobenzyl) piperidine-4-carboxamide (200 mg, 733 μmol, 1.0 eq, HCl salt) was added in portions. The resulting mixture was stirred at 20° C. for 16 h. LCMS indicated that the starting material was completely consumed, and the desired mass was detected. The reaction mixture was poured into H₂O (5.0 mL) and extracted with DCM (3.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 40 mL/min; gradient: 15%-45% B over 8 min; mobile phase A: 10 mM aqueous NH₄HCO₃, mobile phase B: acetonitrile). N-(3-Fluorobenzyl)-1-(1-(isoquinolin-5-yl)ethyl)piperidine-4-carboxamide (5.60 mg, 14.3 μmol, 2% yield) was obtained as a white solid. M+H⁺=392.2 (LCMS); ¹H NMR (400 MHZ, DMSO-d₆) δ 9.29 (s, 1H), 8.50 (d, J=6.1 Hz, 1H), 8.27 (br d, J=6.0 Hz, 2H), 8.00 (d, J=8.0 Hz, 1H), 7.78 (d, J=7.1 Hz, 1H), 7.64 (s, 1H), 7.40-7.24 (m, 1H), 7.09-6.93 (m, 3H), 4.24 (d, J=6.0 Hz, 2H), 4.19-4.13 (m, 1H), 3.06-2.96 (m, 1H), 2.82-2.73 (m, 1H), 2.19-2.09 (m, 1H), 2.07-1.96 (m, 2H), 1.76-1.67 (m, 1H), 1.59 (br s, 3H), 1.40 (d, J=6.6 Hz, 3H).

### Example 370: N-(3-Fluorobenzyl)-1-(1-(3-methoxynaphthalen-1-yl)ethyl)piperidine-4-carboxamide (Compound 524)

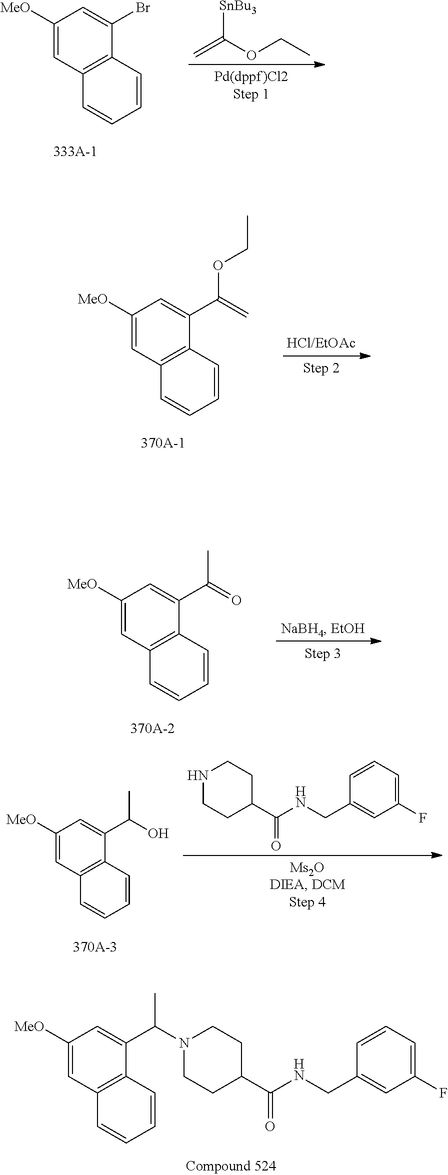

### Step 1: 1-(1-Ethoxyvinyl)-3-methoxynaphthalene (370A-1)

To a solution of 1-bromo-3-methoxynaphthalene (100 mg, 422 μmol, 1.0 eq) in toluene (2.0 mL) were added tributyl(1-ethoxyvinyl) stannane (305 mg, 844 μmol, 2.0 eq) and Pd (dppf) C12 (30.9 mg, 42.2 μmol, 0.1 eq). The mixture was stirred at 110° C. for 16 h under a N₂ atmosphere. LCMS indicated that the starting material remained, and the desired mass was detected. The mixture was allowed to cool to room temperature, poured into water (30 mL) and extracted with EtOAc (30 mL×3). The combined organic layers were dried over Na₂SO₄, filtered, and concentrated under vacuum to give a residue which was purified by preparative TLC (EtOAc/petroleum ether=1/10, R_{f}=0.6). 1-(1-Ethoxyvinyl)-3-methoxynaphthalene (82.2 mg, 360 μmol, 85% yield) was obtained as a white solid. M+H⁺=229.2 (LCMS).

### Step 2: 1-(3-Methoxynaphthalen-1-yl)ethanone (370A-2)

To a solution of HCl/EtOAc (4 M, 1.0 mL) was added 1-(1-ethoxyvinyl)-3-methoxynaphthalene (82.2 mg, 360 μmol, 1.0 eq). The mixture was stirred at 20° C. for 1 h. LCMS indicated that the starting material was completely consumed, and the desired mass was detected. The mixture was treated with water (30 mL) and extracted with EtOAc (10 mL×3). The combined organic layers were dried over Na₂SO₄, filtered, and concentrated under vacuum to give a residue which was purified by preparative TLC (EtOAc/petroleum ether=1/10, R_{f}=0.59). 1-(3-Methoxynaphthalen-1-yl)ethanone (40.1 mg, 178 μmol, 50% yield) was obtained as a yellow oil. M+H⁺=201.1 (LCMS).

### Step 3: 1-(3-Methoxynaphthalen-1-yl)ethanol (370A-3)

To a solution of 1-(3-methoxynaphthalen-1-yl)ethanone (100 mg, 499 μmol, 1.0 eq) in EtOH (2.0 mL) was added NaBH₄ (20.0 mg, 529 μmol, 1.0 eq). The mixture was stirred at 20° C. for 1 h. LCMS indicated that the starting material was completely consumed, and the desired mass was detected. The mixture was treated with water (10 mL) and extracted with EtOAc (10 mL×3). The combined organic layers were dried over Na₂SO₄, filtered, and concentrated under vacuum to give a crude product 1-(3-methoxynaphthalen-1-yl)ethanol (110 mg) as a yellow oil. M+H⁺−18=185.2 (LCMS).

### Step 4: N-(3-Fluorobenzyl)-1-(1-(3-methoxynaphthalen-1-yl)ethyl)piperidine-4-carboxamide (Compound 524)

To a solution of 1-(3-methoxynaphthalen-1-yl)ethanol (40.0 mg, 198 μmol, 1.0 eq) in DCM (2.0 mL) were added DIEA (102 mg, 791 μmol, 4.0 eq) and methylsulfonyl methanesulfonate (34.5 mg, 198 μmol, 1.0 eq) dropwise at 0° C. The mixture was stirred at 0° C. for 30 min, then N-(3-fluorobenzyl) piperidine-4-carboxamide (46.7 mg, 198 μmol, 1.0 eq) was added and the mixture was stirred at 40° C. for 16 h. LCMS indicated that the starting material was completely consumed, and the desired mass was detected. The reaction mixture was allowed to cool to room temperature and concentrated under vacuum at 30° C. to give a residue which was purified by preparative HPLC (Phenomenex C18 column (75×30 mm, 3 μm); flow rate: 25 mL/min; gradient: 50%-75% B over 8 min; mobile phase A: 10 mM aqueous NH₄HCO₃, mobile phase B: acetonitrile). N-(3-Fluorobenzyl)-1-(1-(3-methoxynaphthalen-1-yl)ethyl)piperidine-4-carboxamide (2.97 mg, 6.50 μmol, 3% yield) was obtained as a pale yellow gum. M+H⁺=421.2 (LCMS); ¹H NMR (400 MHZ, DMSO-d₆) δ 8.39-8.24 (m, 2H), 7.81 (d, J=7.9 Hz, 1H), 7.43 (t, J=7.4 Hz, 1H), 7.38-7.27 (m, 2H), 7.23-7.15 (m, 2H), 7.08-6.90 (m, 3H), 4.24 (d, J=6.0 Hz, 2H), 4.09 (br d, J=6.6 Hz, 1H), 3.86 (s, 3H), 3.07 (br d, J=10.4 Hz, 1H), 2.78 (br d, J=11.9 Hz, 1H), 2.20-2.09 (m, 1H), 2.00 (br d, J=11.4 Hz, 2H), 1.77-1.67 (m, 1H), 1.65-1.56 (m, 2H), 1.55 (br d, J=3.3 Hz, 1H), 1.37 (d, J=6.6 Hz, 3H).

### Example 371: (R)-4-Methyl-3-(((1-(naphthalen-1-yl)ethyl)amino)methyl) aniline (Compound 106)

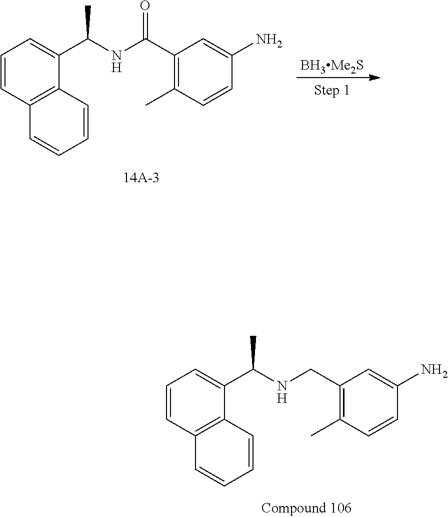 [SMILES: Cc1ccc(N)cc1C(=O)N[C@H](C)c1cccc2ccccc12 ; Cc1ccc(N)cc1CN[C@H](C)c1cccc2ccccc12]

### Step 1: (R)-4-Methyl-3-(((1-(naphthalen-1-yl)ethyl)amino)methyl) aniline (Compound 106)

To a solution of (R)-5-amino-2-methyl-N-(1-(naphthalen-1-yl)ethyl)benzamide (50.0 mg, 164 μmol, 1.0 eq) in THF (2.0 mL) was added BH₃-Me₂S (10 M, 164 μL, 10 eq) at 0° C. The mixture was stirred at 60° C. for 12 h. LCMS indicated that the starting material was completely consumed, and the desired mass was detected. The reaction mixture was allowed to cool to room temperature, poured into H₂O (5.0 mL) and extracted with EtOAc (1.0 mL×5). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 50%-70% B over 8 min; mobile phase A: 10 mM aqueous NH₄HCO₃, mobile phase B: acetonitrile). (R)-4-Methyl-3-(((1-(naphthalen-1-yl)ethyl)amino)methyl) aniline (18.2 mg, 60.2 μmol, 37% yield) was obtained as a yellow oil. M+H⁺=291.2 (LCMS); ¹H NMR (400 MHZ, CDCl₃) δ 8.19 (br d, J=7.8 Hz, 1H), 7.95-7.86 (m, 1H), 7.78 (br dd, J=5.4, 6.8 Hz, 2H), 7.59-7.45 (m, 3H), 6.94 (d, J=8.0 Hz, 1H), 6.71 (s, 1H), 6.53 (dd, J=2.0, 7.9 Hz, 1H), 4.73 (q, J=6.5 Hz, 1H), 3.73-3.58 (m, 2H), 2.16 (s, 3H), 1.55 (d, J=6.5 Hz, 3H).

### Example 372: N-((1H-indazol-5-yl)methyl)-1-(naphthalen-1-yl)cyclopropan-1-amine (Compound 178)

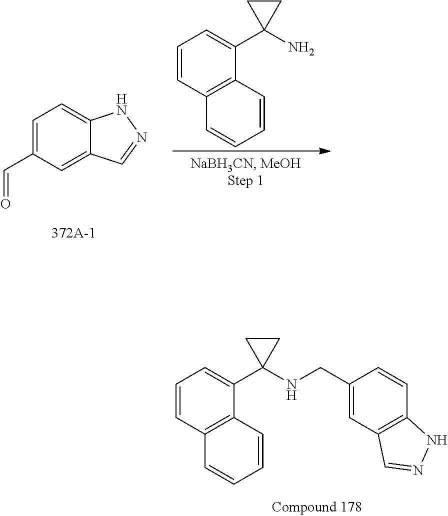 [SMILES: O=Cc1ccc2[nH]ncc2c1 ; NC1(c2cccc3ccccc23)CC1 ; c1ccc2c(C3(NCc4ccc5[nH]ncc5c4)CC3)cccc2c1]

### Step 1: N-((1H-Indazol-5-yl)methyl)-1-(naphthalen-1-yl)cyclopropan-1-amine (Compound 178)

To a stirred solution of 1H-indazole-5-carbaldehyde (100 mg, 684 μmol, 1.0 eq) and 1-(naphthalen-1-yl)cyclopropan-1-amine (125 mg, 684 μmol, 1.0 eq) in MeOH (5.0 mL) was added NaBH₃CN (86.0 mg, 1.37 mmol, 2.0 eq). The mixture was stirred at 20° C. for 12 h. LCMS indicated that the starting material was completely consumed. The mixture was treated with water (10 mL) and extracted with DCM (5.0 mL×5). The combined organic layers were dried over Na₂SO₄, filtered, and concentrated under vacuum to give a residue which was purified by preparative HPLC (Waters Xbridge Prep BEH C18 column (100×30 mm, 10 μm); flow rate: 25 mL/min; gradient: 50%-70% B over 8 min; mobile phase A: 10 mM aqueous NH₄HCO₃, mobile phase B: acetonitrile). N-((1H-Indazol-5-yl)methyl)-1-(naphthalen-1-yl)cyclopropan-1-amine (200 mg, 619 μmol, 90% yield) was obtained as white solid. M+H⁺=314.0 (LCMS); ¹H NMR (400 MHZ, CDCl₃) δ 10.34-9.65 (m, 1H), 8.55-8.39 (m, 1H), 7.98-7.90 (m, 2H), 7.85-7.78 (m, 1H), 7.61-7.43 (m, 5H), 7.38-7.31 (m, 1H), 7.23-7.17 (m, 1H), 3.77-3.61 (m, 2H), 1.35-1.16 (m, 2H), 1.12-0.94 (m, 2H).

### Example 373: N-((1H-Indazol-5-yl)methyl)-N-methyl-1-(naphthalen-1-yl)cyclopropane amine (Compound 184)

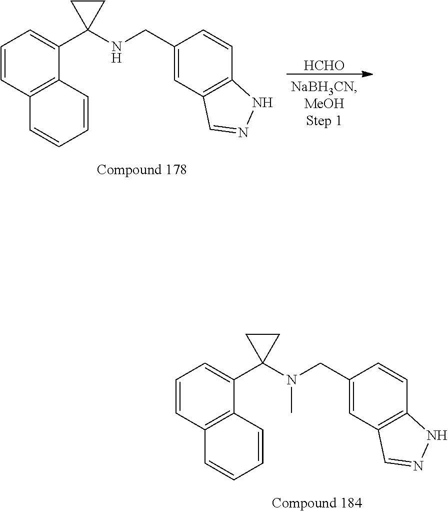 [SMILES: c1ccc2c(C3(NCc4ccc5[nH]ncc5c4)CC3)cccc2c1 ; "HCHO" ; CN(Cc1ccc2[nH]ncc2c1)C1(c2cccc3ccccc23)CC1]

### Step 1: N-((1H-Indazol-5-yl)methyl)-N-methyl-1-(naphthalen-1-yl)cyclopropanamine (Compound 184)

To a stirred solution of N-(1H-indazol-5-ylmethyl)-1-(1-naphthyl)cyclopropanamine (140 mg, 447 μmol, 1.0 eq) in MeOH (5.0 mL) was added formaldehyde (72.5 mg, 893 μmol, 66.5 μL, 37% purity in water, 2.0 eq), followed by NaBH₃CN (56.1 mg, 893 μmol, 2.0 eq). The mixture was stirred at 20° C. for 30 min. LCMS indicated that the starting material was completely consumed. The mixture was treated with water (10 mL) and extracted with DCM (5.0 mL×5). The combined organic layers were dried over Na₂SO₄, filtered, and concentrated under vacuum to give a residue which was purified by preparative HPLC (Phenomenex Luna C18 column (75×30 mm, 3 μm); flow rate: 25 mL/min; gradient: 10%-40% B over 8 min; mobile phase A: 0.1% aqueous TFA, mobile phase B: acetonitrile). N-((1H-Indazol-5-yl)methyl)-N-methyl-1-(naphthalen-1-yl)cyclopropanamine (42.2 mg, 127 μmol, 28% yield) was obtained as white solid. M+H⁺=328.1 (LCMS); ¹H NMR (400 MHZ, CD₃OD) δ 8.69-8.58 (m, 1H), 8.06-7.94 (m, 3H), 7.89-7.82 (m, 1H), 7.71-7.48 (m, 5H), 7.40-7.32 (m, 1H), 4.26-4.12 (m, 1H), 4.38-3.95 (m, 2H), 2.56-2.39 (m, 3H), 1.85-1.64 (m, 2H), 1.51-1.28 (m, 2H).

### Example 374: N, 1,5-Trimethyl-N-(1-(naphthalen-1-yl)cyclopropyl)-1H-indole-6-carboxamide (Compound 208)

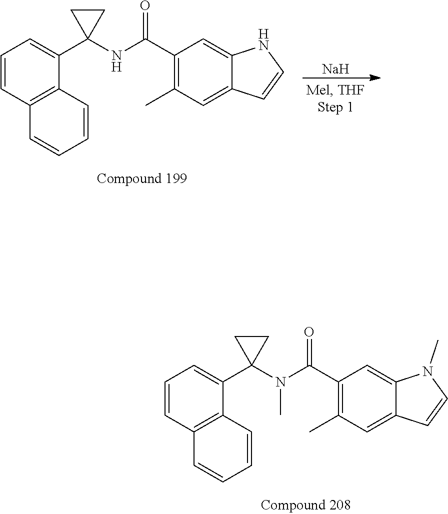

### Step 1: N,1,5-Trimethyl-N-(1-(naphthalen-1-yl)cyclopropyl)-1H-indole-6-carboxamide (Compound 208)

To a solution of 5-methyl-N-(1-(naphthalen-1-yl)cyclopropyl)-1H-indole-6-carboxamide (50.0 mg, 147 μmol, 1.0 eq) in THF (2.0 mL) was added sodium hydride (11.8 mg, 294 μmol, 60% purity, 2.0 eq) at 0° C., followed by Mel (20.9 mg, 147 μmol, 9.14 μL, 1.0 eq). The mixture was stirred at 20° C. for 2 h. LCMS indicated that the starting material completely consumed, and the desired mass was detected. The reaction mixture was poured into H₂O (5.0 mL) and extracted with EtOAc (5.0 mL×2). The combined organic layers were dried over Na₂SO₄, filtered, and concentrated under vacuum to give a residue which was purified by preparative HPLC (Phenomenex Luna C18 (80×30 mm, 3 μm); flow rate: 25 mL/min; gradient: 40%-80% B over 8 min; mobile phase A: 0.04% aqueous HCl, mobile phase B: acetonitrile). N,1,5-Trimethyl-N-(1-(naphthalen-1-yl)cyclopropyl)-1H-indole-6-carboxamide (17.0 mg, 46.1 μmol, 31% yield, HCl salt) was obtained as a white solid. M+H⁺=339.1 (LCMS); ¹H NMR (400 MHz, CDCl₃) δ 9.37-9.12 (m, 1H), 8.02-7.94 (m, 1H), 7.93-7.81 (m, 2H), 7.69-7.59 (m, 1H), 7.58-7.44 (m, 2H), 7.37-7.31 (m, 1H), 7.02-6.99 (m, 1H), 6.99-6.97 (m, 1H), 6.39-6.34 (m, 1H), 3.69 (s, 3H), 2.85 (s, 3H), 2.10 (s, 3H), 1.92-1.72 (m, 2H), 1.58-1.41 (m, 2H).

### Example 375: N-(3-Fluorobenzyl)-1-(1-(naphthalen-1-yl)cyclopropyl) piperidine-4-carboxamide (Compound 179)

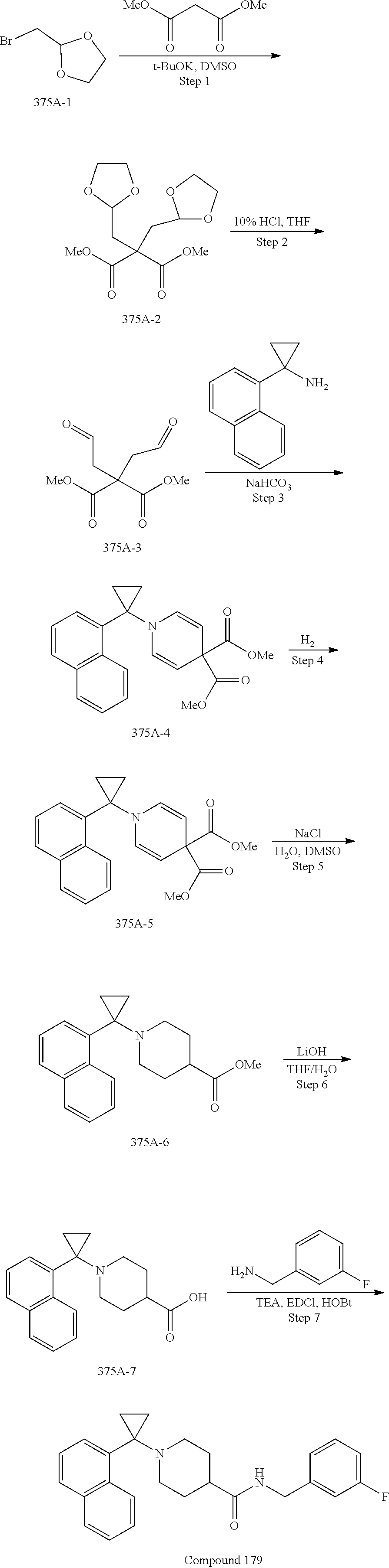

### Step 1: Dimethyl 2,2-bis((1,3-dioxolan-2-yl)methyl)malonate (375A-2)

A mixture of dimethyl malonate (5.01 g, 37.9 mmol, 4.35 mL, 1.0 eq) and t-BuOK (5.10 g, 45.5 mmol, 1.2 eq) in DMSO (100 mL) was stirred at 20° C. for 1 h. 2-(Bromomethyl)-1,3-dioxolane (7.60 g, 45.5 mmol, 4.66 mL, 1.2 eq) was added, and the mixture was stirred at 80° C. for 12 h. The solution was cooled to room temperature, 1-BuOK (5.10 g, 45.5 mmol, 1.2 eq) was added, and the mixture was stirred at room temperature for 1 h. 2-(Bromomethyl)-1,3-dioxolane (7.60 g, 45.5 mmol, 4.66 mL, 1.2 eq) was added again, and the solution was stirred at 80° C. for another 12 h. LCMS indicated that the starting material was completely consumed, and the desired mass was detected. The mixture was poured into H₂O (100 mL) and extracted with EtOAc (40 mL×3). The combined organic layers were dried over Na₂SO₄, filtered, and concentrated under vacuum to give a residue which was purified by flash silica gel chromatography using a gradient of EtOAc/petroleum ether from 1/100 to 3/10. Dimethyl 2,2-bis((1,3-dioxolan-2-yl)methyl)malonate (5.00 g, 16.4 mmol, 43% yield) was obtained as a colorless oil. M+H⁺=305.2 (LCMS); ¹H NMR (400 MHZ, CDCl₃) δ 5.02 (t, J=4.8 Hz, 2H), 3.95-3.88 (m, 4H), 3.83-3.77 (m, 4H), 3.74-3.68 (m, 6H), 2.45 (d, J=4.9 Hz, 4H).

### Step 2: Dimethyl 2,2-bis(2-oxoethyl)malonate (375A-3)

To a solution of dimethyl 2,2-bis((1,3-dioxolan-2-yl)methyl)malonate (2.00 g, 6.57 mmol, 1.0 eq) in THF (40 mL) was added HCl (10% purity in water, 40.0 mL). The mixture was stirred at 20° C. for 12 h. TLC indicated that the starting material was completely consumed. Dimethyl 2,2-bis(2-oxoethyl)malonate was obtained as a mixture (40 mL), which was used in the next step without any further purification.

### Step 3: Dimethyl 1-(1-(naphthalen-1-yl)cyclopropyl)pyridine-4,4(1H)-dicarboxylate (375A-4)

To a solution of dimethyl 2,2-bis(2-oxoethyl)malonate (590 mg, 2.73 mmol, 40 mL, 1.0 eq) was added NaHCO₃ (458 mg, 5.46 mmol, 212 μL, 2.0 eq) to adjust the pH to 7, followed by a solution of 1-(naphthalen-1-yl)cyclopropan-1-amine (500 mg, 2.73 mmol, 1.0 eq) in THF (5.0 mL). The mixture was stirred at 20° C. for 12 h. LCMS indicated that the starting material was completely consumed, and the desired mass was detected. The mixture was extracted with DCM (20 mL×3). The combined organic layers were dried over Na₂SO₄, filtered, and concentrated under vacuum to give a residue which was purified by flash silica gel chromatography using a gradient of EtOAc/petroleum ether from 1/100 to 1/10. Dimethyl 1-(1-(naphthalen-1-yl)cyclopropyl)pyridine-4,4(1H)-dicarboxylate (300 mg, 826 μmol, 30% yield) was obtained as a white solid. M+H⁺=364.2 (LCMS).

### Step 4: Dimethyl 1-(1-(naphthalen-1-yl)cyclopropyl) piperidine-4,4-dicarboxylate (375A-5)

To a solution of dimethyl 1-(1-(naphthalen-1-yl)cyclopropyl)pyridine-4,4(1H)-dicarboxylate (150 mg, 413 μmol, 1.0 eq) in MeOH (5.0 mL) was added 10% palladium on carbon (100 mg) under a N₂ atmosphere. The suspension was degassed and purged with H₂ three times. The mixture was stirred at 20° C. for 12 h under a H₂ (50 psi) atmosphere. LCMS indicated that the starting material was completely consumed, and the desired mass was detected. The suspension was filtered through a pad of Celite and the filter cake was washed with MeOH (3.0 mL×5). The combined filtrates were concentrated under vacuum to give a residue which was purified by preparative TLC (petroleum ether/EtOAc=5/1, R_{f}=0.4). Dimethyl 1-(1-(naphthalen-1-yl)cyclopropyl) piperidine-4,4-dicarboxylate (100 mg, 272 μmol, 66% yield) was obtained as a white solid. M+H⁺=368.2 (LCMS).

### Step 5: Methyl 1-(1-(naphthalen-1-yl)cyclopropyl) piperidine-4-carboxylate (375A-6)

To a solution of dimethyl 1-(1-(naphthalen-1-yl)cyclopropyl) piperidine-4,4-dicarboxylate (100 mg, 272 μmol, 1.0 eq) in DMSO (2.0 mL) were added NaCl (79.5 mg, 1.36 mmol, 5.0 eq) and H₂O (24.5 mg, 1.36 mmol, 5.0 eq). The mixture was stirred at 140° C. for 12 h. LCMS indicated that the starting material was completely consumed, and the desired mass was detected. The mixture was poured into H₂O (2.0 mL) and extracted with DCM (2.0 mL×2). The combined organic layers were dried over Na₂SO₄, filtered, and concentrated under vacuum to give the crude methyl 1-(1-(naphthalen-1-yl)cyclopropyl) piperidine-4-carboxylate (80.0 mg), which was used in the next step without any further purification. M+H⁺=310.3 (LCMS).

### Step 6: 1-(1-(Naphthalen-1-yl)cyclopropyl) piperidine-4-carboxylic acid (375A-7)

To a solution of methyl 1-(1-(naphthalen-1-yl)cyclopropyl) piperidine-4-carboxylate (40.0 mg, 129 μmol, 1.0 eq) in a mixture of H₂O (2.0 mL) and THF (5.0 mL) was added LiOH·H₂O (16.3 mg, 388 μmol, 3.0 eq). The mixture was stirred at 20° C. for 12 h. LCMS indicated that the starting material was completely consumed, and the desired mass was detected. The reaction mixture was allowed to cool to room temperature, poured into H₂O (7.0 mL) and washed with MTBE (4.0 mL×2). The aqueous layer was acidified to pH 5 with HCl (1 M aqueous). The product was extracted with EtOAc (3.0 mL×3). The combined organic layers were dried over Na₂SO₄, filtered and concentrated under vacuum to give 1-(1-(naphthalen-1-yl)cyclopropyl) piperidine-4-carboxylic acid (30.0 mg) as a white solid, which was used in the next step without any further purification. M+H⁺=296.1 (LCMS).

### Step 7: N-(3-Fluorobenzyl)-1-(1-(naphthalen-1-yl)cyclopropyl) piperidine-4-carboxamide (Compound 179)

To a solution of 1-(1-(naphthalen-1-yl)cyclopropyl) piperidine-4-carboxylic acid (30.0 mg, 102 μmol, 1.0 eq) and (3-fluorophenyl)methanamine (14.0 mg, 112 μmol, 12.7 μL, 1.1 eq) in DCM (2.0 mL) were added TEA (30.8 mg, 305 μmol, 42 μL, 3.0 eq), EDCI (38.9 mg, 203 μmol, 2.0 eq) and HOBt (27.5 mg, 203 μmol, 2.0 eq). The mixture was stirred at 20° C. for 12 h. LCMS indicated that the starting material was completely consumed, and the desired mass was detected. The mixture was poured into H₂O (2.0 mL) and extracted with DCM (2.0 mL×2). The combined organic layers were dried over Na₂SO₄, filtered, and concentrated under vacuum to give a residue which was purified by preparative HPLC (Phenomenex Luna C18 column (150×30 mm, 5 μm); flow rate: 25 mL/min; gradient: 15%-45% B over 8 min; mobile phase A: 0.1% aqueous TFA, mobile phase B: acetonitrile). N-(3-Fluorobenzyl)-1-(1-(naphthalen-1-yl)cyclopropyl) piperidine-4-carboxamide (6.31 mg, 12.2 μmol, 12% yield, TFA salt) was obtained as a yellow gum. M+H⁺=403.1 (LCMS); ¹H NMR (400 MHZ, CD₃OD) δ 8.50 (d, J=8.6 Hz, 1H), 8.42-8.32 (m, 1H), 8.14-8.07 (m, 1H), 8.04-7.98 (m, 1H), 7.94-7.86 (m, 1H), 7.75-7.68 (m, 1H), 7.66-7.58 (m, 2H), 7.35-7.24 (m, 1H), 7.09-7.01 (m, 1H), 7.00-6.87 (m, 2H), 4.41-4.25 (m, 2H), 4.23-3.80 (m, 2H), 3.14-2.94 (m, 1H), 2.80-2.60 (m, 1H), 2.31-2.16 (m, 1H), 2.14-1.85 (m, 5H), 1.80-1.25 (m, 4H).

### Example 376: N-(5-(2-(Dimethylamino)ethoxy)-2-methylphenyl)-2-(naphthalen-1-yl) propanamide (Compound 577)

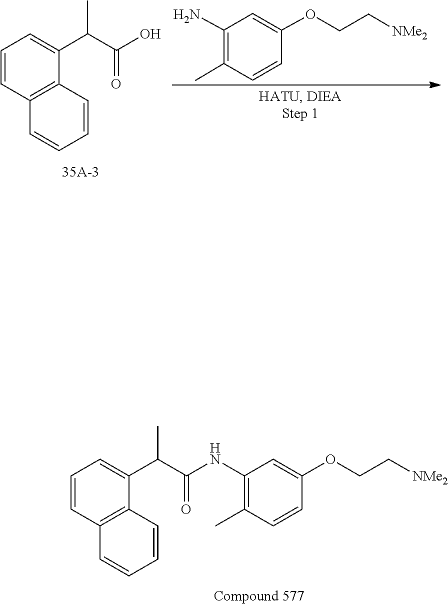 [SMILES: CC(C(=O)O)c1cccc2ccccc12 ; Cc1ccc(OCCN(C)C)cc1N ; Cc1ccc(OCCN(C)C)cc1NC(=O)C(C)c1cccc2ccccc12]

### Step 1: N-(5-(2-(Dimethylamino)ethoxy)-2-methylphenyl)-2-(naphthalen-1-yl)propanamide (Compound 577)

To a stirred solution of 2-(naphthalen-1-yl) propanoic acid (65.0 mg, 325 μmol, 1.0 eq) and 5-(2-(dimethylamino)ethoxy)-2-methylaniline (69.4 mg, 357 μmol, 1.1 eq) in DMF (3.5 mL) was added DIEA (126 mg, 974 μmol, 170 μL, 3.0 eq), followed by HATU (370 mg, 974 μmol, 3.0 eq). The mixture was stirred at 20° C. for 16 h. LCMS indicated that the starting material was completely consumed, and the desired compound was detected. The mixture was poured into H₂O (5 mL) and extracted with EtOAc (5.0 mL×4). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 10%-40% B over 8 min; mobile phase A: 0.04% aqueous HCl, mobile phase B: acetonitrile). N-(5-(2-(Dimethylamino)ethoxy)-2-methylphenyl)-2-(naphthalen-1-yl) propanamide (36.2 mg, 85.4 μmol, 26% yield, HCl salt) was obtained as a yellow solid. M+H⁺=377.1 (LCMS); ¹H NMR (400 MHZ, DMSO-d₆) δ 9.91-9.73 (m, 1H), 9.52-9.43 (m, 1H), 8.37-8.28 (m, 1H), 8.00-7.93 (m, 1H), 7.89-7.82 (m, 1H), 7.63-7.48 (m, 4H), 7.15-7.08 (m, 2H), 6.77-6.68 (m, 1H), 4.83-4.69 (m, 1H), 4.24 (t, J=4.8 Hz, 2H), 3.46 (q, J=4.9 Hz, 2H), 2.82 (d, J=4.6 Hz, 6H), 2.04 (s, 3H), 1.60 (d, J=7.0 Hz, 3H).

### Example 377: N-(5-(2-(Dimethylamino)ethoxy)-2-methylphenyl)-1-(naphthalen-1-yl)cyclopropane-1-carboxamide (Compound 580)

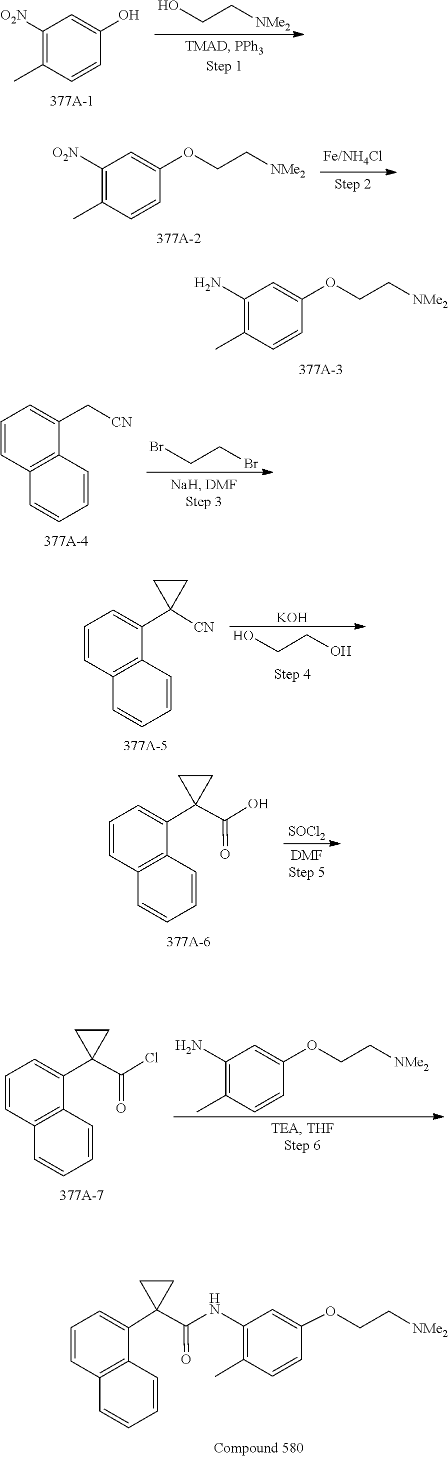

### Step 1: N,N-Dimethyl-2-(4-methyl-3-nitrophenoxy)ethan-1-amine (377A-2)

To a mixture of 4-methyl-3-nitrophenol (1.50 g, 9.80 mmol, 1.0 eq) and 2-(dimethylamino)ethanol (873 mg, 9.80 mmol, 98.9 μL, 1.0 eq) in toluene (80 mL) were added TMAD (5.06 g, 29.4 mmol, 3.0 eq) and PPh₃ (7.71 g, 29.4 mmol, 3.0 eq). The mixture was degassed and purged with N₂ three times. The resulting mixture was stirred at 100° C. for 12 h under a N₂ atmosphere. LCMS indicated that the starting material was completely consumed, and the desired mass was detected. The reaction mixture was allowed to cool to room temperature, poured into H₂O (80 mL) and extracted with EtOAc (30 mL×3). The combined organic layers were dried over Na₂SO₄, filtered, and concentrated under vacuum to give a residue which was purified by flash silica gel chromatography using a gradient of EtOAc/petroleum ether from 0/1 to 1/2. N,N-Dimethyl-2-(4-methyl-3-nitrophenoxy)ethan-1-amine (1.80 g, 8.03 mmol, 82% yield) was obtained as a brown solid. M+H⁺=225.1 (LCMS).

### Step 2: 5-(2-(Dimethylamino)ethoxy)-2-methylaniline (377A-3)

To a mixture of N,N-dimethyl-2-(4-methyl-3-nitrophenoxy)ethan-1-amine (1.00 g, 4.46 mmol, 1.0 eq) in a mixture of MeOH (15 mL) and H₂O (3.0 mL) were added iron powder (1.25 g, 22.3 mmol, 5.0 eq) and NH₄Cl (1.19 g, 22.3 mmol, 5.0 eq). The mixture was degassed and purged with N₂ three times. The resulting mixture was stirred at 100° C. for 12 h under a N₂ atmosphere. LCMS indicated that the starting material was completely consumed, and the desired mass was detected. The reaction mixture was allowed to cool to room temperature, poured into H₂O (20 mL) and extracted with EtOAc (10 mL×3). The combined organic layers were dried over Na₂SO₄, filtered, and concentrated under vacuum to give a residue which was triturated with EtOAc (2.0 mL). 5-(2-(Dimethylamino)ethoxy)-2-methylaniline (500 mg, crude) was obtained as a brown solid, which was used in the next step without any further purification. M+H⁺=219.1 (LCMS).

### Step 3: 1-(Naphthalen-1-yl)cyclopropane-1-carbonitrile (377A-5)

To a solution of 2-(1-naphthyl) acetonitrile (1.67 g, 9.99 mmol, 1.0 eq) in DMF (10 mL) was added NaH (1.20 g, 30.0 mmol, 60% purity, 3.0 eq) at −30° C. under a N₂ atmosphere. The resulting mixture was stirred at the same temperature for 1 h, then a solution of 1,2-dibromoethane (3.75 g, 20.0 mmol, 1.5 mL, 2.0 eq) in DMF (10 mL) was added dropwise. The resulting mixture was warmed slowly to 25° C. and stirred at the same temperature for 30 min. LCMS indicated that the starting material was completely consumed, and the desired mass was detected. The mixture was quenched with saturated aqueous NH₄Cl (30 mL) and extracted with EtOAc (30 mL×3). The combined organic layers were dried over Na₂SO₄, filtered, and concentrated under vacuum to give a residue which was purified by flash silica gel chromatography using a gradient of EtOAc/petroleum ether from 0/1 to 1/8. 1-(Naphthalen-1-yl)cyclopropane-1-carbonitrile (1.00 g, 5.17 mmol, 52% yield) was obtained as a white solid. M+H⁺=194.1 (LCMS).

### Step 4: 1-(Naphthalen-1-yl)cyclopropane-1-carboxylic acid (377A-6)

To a solution of (400 mg, 2.07 mmol, 1.0 eq) in ethane-1,2-diol (5.0 mL) was added KOH (1.16 g, 20.7 mmol, 10 eq). The resulting mixture was stirred at 190° C. for 3 h. TLC indicated that the starting material was completely consumed. The reaction mixture was allowed to cool to room temperature, poured into H₂O (7.0 mL), and washed with MTBE (4.0 mL×2). The aqueous layer was acidified to pH 5 with HCl (1 M aqueous). The product was extracted with EtOAc (3.0 mL×3). The combined organic layers were dried over Na₂SO₄, filtered and concentrated under vacuum to give the crude 1-(naphthalen-1-yl)cyclopropane-1-carboxylic acid (300 mg, 1.41 mmol, 68% yield) as a brown solid, which was used in the next step without any further purification. ¹H NMR (400 MHZ, DMSO-d₆) δ 8.09 (d, J=8.3 Hz, 1H), 7.94 (d, J=7.9 Hz, 1H), 7.87-7.81 (m, 1H), 7.59-7.41 (m, 4H), 1.75-1.55 (m, 2H), 1.31-1.14 (m, 2H).

### Step 5: 1-(Naphthalen-1-yl)cyclopropane-1-carbonyl chloride (377A-7)

To a solution of 1-(naphthalen-1-yl)cyclopropane-1-carboxylic acid (100 mg, 471 μmol, 1.0 eq) in SOCl₂ (224 mg, 1.88 mmol, 137 μL, 4.0 eq) was added DMF (3.44 mg, 47.0 μmol, 3.63 μL, 0.1 eq). The resulting mixture was stirred at 90° C. for 2 h. LCMS indicated that the starting material was completely consumed, and the desired mass was detected. The reaction mixture was allowed to cool to room temperature and concentrated under vacuum to give a crude 1-(naphthalen-1-yl)cyclopropane-1-carbonyl chloride (100 mg) as a brown oil, which was used in the next step without any further purification. M+H⁺=227.1 (LCMS, quenched by MeOH for testing).

### Step 6: N-(5-(2-(Dimethylamino)ethoxy)-2-methylphenyl)-1-(naphthalen-1-yl)cyclopropane-1-carboxamide (Compound 580)

To a mixture of 5-(2-(dimethylamino)ethoxy)-2-methylaniline (75.8 mg, 390 μmol, 1.0 eq) in THF (2.0 mL) was added TEA (79.0 mg, 780 μmol, 110 μL, 2.0 eq), then a solution of 1-(naphthalen-1-yl)cyclopropanecarbonyl chloride (90.0 mg, 390 μmol, 1.0 eq) in THF (1.0 mL) was added dropwise. The resulting mixture was stirred at 20° C. for 1 h. LCMS indicated that the starting material was completely consumed, and the desired mass was detected. The mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 20%-35% B over 8 min; mobile phase A: 0.04% aqueous HCl, mobile phase B: acetonitrile). N-(5-(2-(Dimethylamino)ethoxy)-2-methylphenyl)-1-(naphthalen-1-yl)cyclopropane-1-carboxamide (11.3 mg, 28.7 μmol, 7% yield, HCl salt) was obtained as a brown solid. M+H⁺=389.1 (LCMS); ¹H NMR (400 MHZ, DMSO-d₆) δ 9.66 (s, 1H), 8.19 (d, J=8.4 Hz, 1H), 8.00 (br d, J=16.1 Hz, 2H), 7.73 (d, J=6.5 Hz, 1H), 7.54-7.65 (m, 4H), 7.29 (d, J=2.5 Hz, 1H), 6.95 (d, J=8.4 Hz, 1H), 6.62 (dd, J=2.6, 8.3 Hz, 1H), 4.16-4.23 (m, 2H), 3.46 (br d, J=5.1 Hz, 2H), 2.83 (s, 6H), 1.74 (br d, J=2.3 Hz, 2H), 1.21-1.30 (m, 2H), 1.34-1.40 (m, 3H).

### Example 378: (5-(Azetidin-3-ylamino)-2-methylphenyl)(5-(5-(pyrrolidin-1-ylmethyl)thiophen-2-yl)isoindolin-2-yl)methanone (Compound 111)

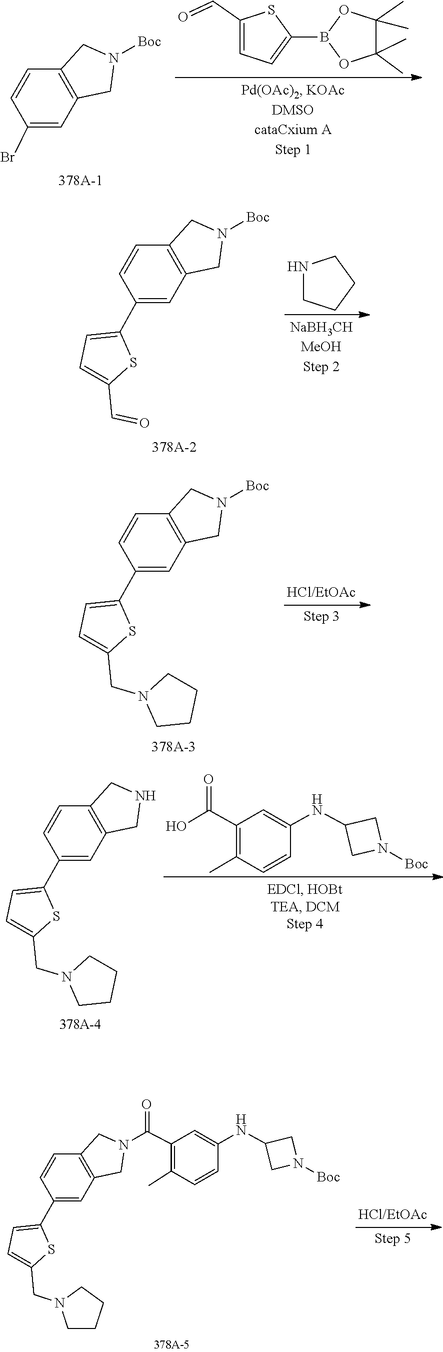

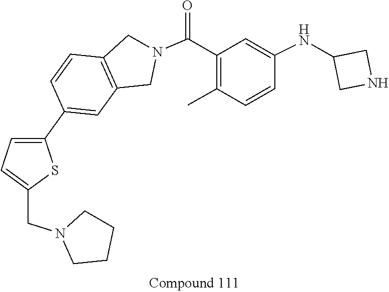 [SMILES: Cc1ccc(NC2CNC2)cc1C(=O)N1Cc2ccc(-c3ccc(CN4CCCC4)s3)cc2C1]

### Step 1: tert-Butyl 5-(5-formylthiophen-2-yl)isoindoline-2-carboxylate (378A-2)

To a stirred solution of tert-butyl 5-bromoisoindoline-2-carboxylate (200 mg, 671 μmol, 1.0 eq) in DMSO (10 mL) were added 5-(4,4,5,5-tetramethyl-1,3,2-dioxaborolan-2-yl)thiophene-2-carbaldehyde (192 mg, 805 μmol, 1.2 eq), Pd(OAc)₂ (15.1 mg, 67.1 μmol, 0.1 eq), cataCxium A (48.1 mg, 134.15 μmol, 0.2 eq), and KOAc (198 mg, 2.01 mmol, 3.0 eq). The mixture was degassed and purged with N₂ three times. The mixture was stirred at 80° C. for 16 h under a N₂ atmosphere. TLC indicated that the starting material was completely consumed. The reaction mixture was allowed to cool to room temperature, treated with H₂O (20 mL) and extracted with EtOAc (10 mL×3). The combined organic layers were dried over Na₂SO₄, filtered, and concentrated under vacuum to give a residue. The residue was triturated from TBME (5 mL) and filtered. The cake was dried under vacuum to give tert-butyl 5-(5-formyl-2-thienyl)isoindoline-2-carboxylate (150 mg, 455 μmol, 68% yield) as an off-white solid.

### Step 2: tert-Butyl 5-(5-(pyrrolidin-1-ylmethyl)thiophen-2-yl)isoindoline-2-carboxylate (378A-3)

To a stirred solution of tert-butyl 5-(5-formyl-2-thienyl)isoindoline-2-carboxylate (120 mg, 364 μmol, 1.0 eq) and pyrrolidine (51.8 mg, 729 μmol, 2.0 eq) in MeOH (15 mL) was added NaBH₃CN (68.7 mg, 1.09 mmol, 3.0 eq) under a N₂ atmosphere. The mixture was stirred at 20° C. for 12 h. TLC indicated that the starting material was completely consumed. The mixture was poured into water (20 mL) and extracted with DCM (15 mL×2). The combined organic layers were dried over Na₂SO₄, filtered, and concentrated under vacuum to give a residue. The residue was treated with TBME (5.0 mL) and a precipitate was formed. The mixture was filtered and the cake was dried under vacuum to give the crude tert-butyl 5-(5-(pyrrolidin-1-ylmethyl)thiophen-2-yl)isoindoline-2-carboxylate (150 mg, crude) as a yellow solid.

### Step 3: 5-(5-(Pyrrolidin-1-ylmethyl)thiophen-2-yl)isoindoline (378A-4)

To a stirred solution of tert-butyl 5-(5-(pyrrolidin-1-ylmethyl)thiophen-2-yl)isoindoline-2-carboxylate (170 mg, 442 μmol, 1.0 eq) in EtOAc (10 mL) was added HCl/EtOAc (4 M, 11 mL). The mixture was stirred at 20° C. for 2 h. TLC indicated that the starting material was completely consumed. The reaction mixture was concentrated under vacuum to give the crude 5-(5-(pyrrolidin-1-ylmethyl)thiophen-2-yl)isoindoline (180 mg, HCl Salt) as a yellow gum, which was used in the next step without any further purification.

### Step 4: tert-Butyl 3-((4-methyl-3-(5-(5-(pyrrolidin-1-ylmethyl)thiophen-2-yl)isoindoline-2-carbonyl)phenyl)amino)azetidine-1-carboxylate (378A-5)

To a stirred solution of 5-((1-(tert-butoxycarbonyl)azetidin-3-yl)amino)-2-methylbenzoic acid (172 mg, 561 μmol, 1.0 eq) and 5-(5-(pyrrolidin-1-ylmethyl)thiophen-2-yl)isoindoline (180 mg, 561 μmol, 1.0 eq, HCl Salt) in DCM (10 mL) were added TEA (170 mg, 1.68 mmol, 234 μL, 3.0 eq), EDCI (129 mg, 673 μmol, 1.2 eq) and HOBt (91.0 mg, 673 μmol, 1.2 eq). The mixture was stirred at 20° C. for 12 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue, which was triturated with TMBE (5 mL) and filtered. The cake was dried under vacuum to give the crude tert-butyl 3-((4-methyl-3-(5-(5-(pyrrolidin-1-ylmethyl)thiophen-2-yl)isoindoline-2-carbonyl)phenyl)amino)azetidine-1-carboxylate (300 mg, 524 μmol, 93% yield) as a yellow solid.

### Step 5: (5-(Azetidin-3-ylamino)-2-methylphenyl)(5-(5-(pyrrolidin-1-ylmethyl)thiophen-2-yl)isoindolin-2-yl)methanone (Compound 111)

To a stirred solution of tert-butyl 3-((4-methyl-3-(5-(5-(pyrrolidin-1-ylmethyl)thiophen-2-yl)isoindoline-2-carbonyl)phenyl)amino)azetidine-1-carboxylate (150 mg, 262 μmol, 1.0 eq) in EtOAc (20 mL) was added HCl/EtOAc (4 M, 20 mL). The mixture was stirred at 20° C. for 2 h. LCMS indicated that the starting material was completely consumed, and the desired mass was detected. The mixture was concentrated in vacuum at 25° C. to give a residue, which was purified by preparative HPLC (Phenomenex Luna column (80× 40 mm, 3 μm); flow rate: 40 mL/min; gradient: 10%-34% B over 7 min; mobile phase A: 0.04% aqueous HCl, mobile phase B: acetonitrile). (5-(Azetidin-3-ylamino)-2-methylphenyl)(5-(5-(pyrrolidin-1-ylmethyl)thiophen-2-yl)isoindolin-2-yl)methanone (72.8 mg, 135.85 μmol, 52% yield, HCl salt) was obtained as yellow gum. M+H⁺=473.1 (LCMS); ¹H NMR (400 MHZ, CD₃OD) δ 7.76-7.51 (m, 2H), 7.48-7.25 (m, 3H), 7.24-7.18 (m, 1H), 6.85-6.80 (m, 1H), 6.76-6.72 (m, 1H), 5.03-4.96 (m, 2H), 4.69-4.55 (m, 5H), 4.47-4.36 (m, 2H), 4.14-4.03 (m, 2H), 3.64-3.57 (m, 2H), 3.29-3.19 (m, 2H), 2.27-2.13 (m, 5H), 2.10-1.99 (m, 2H).

### Example 379: 5-(Azetidin-3-ylamino)-2-methyl-N-(4-(5-(pyrrolidin-1-ylmethyl)thiophen-2-yl)benzyl)benzamide (Compound 116)

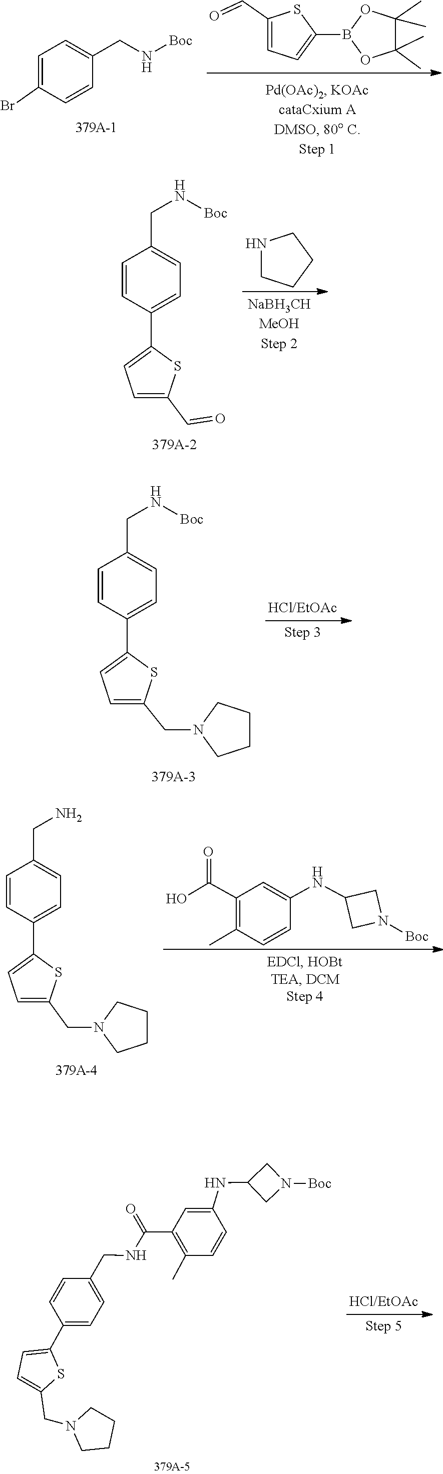

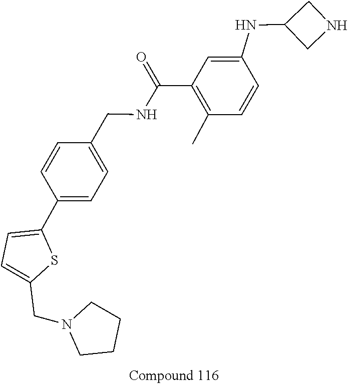 [SMILES: Cc1ccc(NC2CNC2)cc1C(=O)NCc1ccc(-c2ccc(CN3CCCC3)s2)cc1]

### Step 1: tert-Butyl 4-(5-formylthiophen-2-yl)benzylcarbamate (379A-2)

To a stirred solution of tert-butyl 4-bromobenzylcarbamate (200 mg, 699 μmol, 1.0 eq) in DMSO (10 mL) were added 5-(4,4,5,5-tetramethyl-1,3,2-dioxaborolan-2-yl)thiophene-2-carbaldehyde (109 mg, 699 μmol, 1.0 eq), cataCxium A (50.1 mg, 140 μmol, 0.2 eq), Pd(OAc) 2 (15.7 mg, 69.9 μmol, 0.1 eq), and KOAc (206 mg, 2.10 mmol, 3.0 eq) under a N₂ atmosphere. The mixture was stirred at 80° C. for 6 h. TLC indicated that the starting material was completely consumed. The reaction mixture was allowed to cool to room temperature, poured into H₂O (20 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/5. tert-Butyl 4-(5-formylthiophen-2-yl)benzylcarbamate (200 mg, 630 μmol, 90% yield) was obtained as a yellow solid. ¹H NMR (400 MHZ, CDCl₃) δ 9.90 (s, 1H), 7.75 (d, J=3.9 Hz, 1H), 7.65 (d, J=8.3 Hz, 2H), 7.40 (d, J=3.9 Hz, 1H), 7.36 (d, J=8.2 Hz, 2H), 4.91 (br s, 1H), 4.36 (br d, J=5.4 Hz, 2H), 1.48 (s, 9H).

### Step 2: tert-Butyl 4-(5-(pyrrolidin-1-ylmethyl)thiophen-2-yl)benzylcarbamate (379A-3)

To a stirred solution of tert-butyl 4-(5-formylthiophen-2-yl)benzylcarbamate (200 mg, 630 μmol, 1.0 eq) in MeOH (20 mL) was added pyrrolidine (89.6 mg, 1.26 mmol, 105 μL, 2.0 eq), followed by NaBH₃CN (119 mg, 1.89 mmol, 3.0 eq). The mixture was stirred at 20° C. for 16 h. TLC indicated that the starting material was completely consumed. The mixture was poured into water (20 mL) and extracted with DCM (15 mL×2). The combined organic layers were dried over Na₂SO₄, filtered, and concentrated under vacuum to give the crude tert-butyl 4-(5-(pyrrolidin-1-ylmethyl)thiophen-2-yl)benzylcarbamate (250 mg, crude) as a yellow solid, which was used in the next step without any further purification.

### Step 3: (4-(5-(Pyrrolidin-1-ylmethyl)thiophen-2-yl)phenyl)methanamine (379A-4)

To a stirred solution of tert-butyl 4-(5-(pyrrolidin-1-ylmethyl)thiophen-2-yl)benzylcarbamate (250 mg, 671 μmol, 1.0 eq) in EtOAc (15 mL) was added HCl/EtOAc (4 M, 17 mL). The mixture was stirred at 20° C. for 2 h. TLC indicated that the starting material was completely consumed. The mixture was concentrated in vacuum at 25° C. to give the crude (4-(5-(pyrrolidin-1-ylmethyl)thiophen-2-yl)phenyl)methanamine (250 mg, crude HCl salt) as a yellow gum, which was used in the next step without any further purification.

### Step 4: tert-Butyl 3-((4-methyl-3-((4-(5-(pyrrolidin-1-ylmethyl)thiophen-2-yl)benzyl)carbamoyl)phenyl)amino)azetidine-1-carboxylate (379A-5)

To a stirred solution of 5-((1-(tert-butoxycarbonyl)azetidin-3-yl)amino)-2-methylbenzoic acid (200 mg, 653 μmol, 1.0 eq) and (4-(5-(pyrrolidin-1-ylmethyl)thiophen-2-yl)phenyl)methanamine (202 mg, 653 μmol, 1.0 eq, HCl salt) in DCM (10 mL) were added HOBt (106 mg, 783 μmol, 1.2 eq), EDCI (150 mg, 783 μmol, 1.2 eq) and TEA (198 mg, 1.96 mmol, 273 μL, 3.0 eq). The mixture was stirred at 20° C. for 2 h. LCMS indicated that the starting material was completely consumed, and the desired mass was detected. The mixture was treated with water (10 mL) and extracted with DCM (10 mL×2). The combined organic layers were dried over Na₂SO₄, filtered, and concentrated under vacuum to give a residue which was purified by flash silica gel chromatography using a gradient of EtOAc/petroleum ether from 1/100 to 1/1. tert-Butyl 3-((4-methyl-3-((4-(5-(pyrrolidin-1-ylmethyl)thiophen-2-yl)benzyl)carbamoyl)phenyl)amino)azetidine-1-carboxylate (250 mg, 446 μmol, 68% yield) was obtained as a yellow solid.

### Step 5: 5-(Azetidin-3-ylamino)-2-methyl-N-(4-(5-(pyrrolidin-1-ylmethyl)thiophen-2-yl)benzyl)benzamide (Compound 116)

To a stirred solution of tert-butyl 3-((4-methyl-3-((4-(5-(pyrrolidin-1-ylmethyl)thiophen-2-yl)benzyl)carbamoyl)phenyl)amino)azetidine-1-carboxylate (200 mg, 357 μmol, 1.0 eq) in EtOAc (20 mL) was added HCl/EtOAc (4 M, 20 mL). The mixture was stirred at 20° C. for 2 h. LCMS indicated that the starting material was completely consumed. The mixture was concentrated in vacuum at 25° C. to give a residue, which was purified by preparative HPLC (Phenomenex Luna (80×30 mm, 3 μm); flow rate: 25 mL/min; gradient: 5%-25% B over 8 min; mobile phase A: 0.1% aqueous TFA, mobile phase B: acetonitrile). 5-(Azetidin-3-ylamino)-2-methyl-N-(4-(5-(pyrrolidin-1-ylmethyl)thiophen-2-yl)benzyl)benzamide (44.6 mg, 76.1 μmol, 21% yield, TFA salt) was obtained as a yellow gum. M+H⁺=461.1 (LCMS); 1HNMR (400 MHZ, CD₃OD) δ 7.68-7.62 (m, 2H), 7.44 (d, J=8.3 Hz, 2H), 7.40 (d, J=3.8 Hz, 1H), 7.30 (d, J=3.8 Hz, 1H), 7.05 (d, J=7.8 Hz, 1H), 6.61-6.57 (m, 2H), 4.62 (s, 2H), 4.57-4.45 (m, 3H), 4.41-4.33 (m, 2H), 3.95 (dd, J=6.8, 11.3 Hz, 2H), 3.59 (br s, 2H), 3.26 (br s, 2H), 2.25 (s, 5H), 2.10-1.95 (m, 2H).

### Example 380: tert-Butyl (R)-3-((3-((1-(1-(ethylsulfonyl) piperidin-4-yl)ethyl)carbamoyl)-4-methylphenyl)amino)azetidine-1-carboxylate (Compound 161)

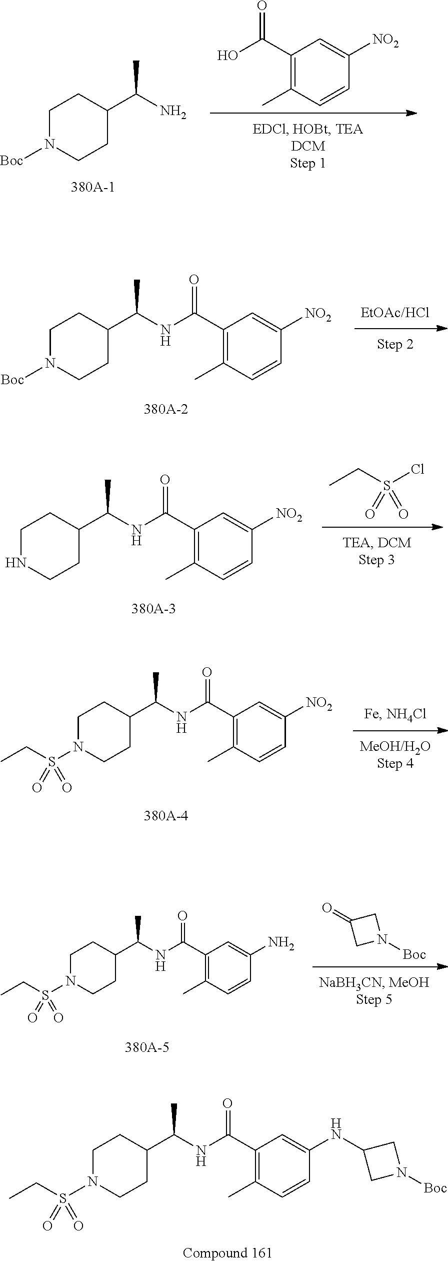

### Step 1: tert-Butyl (R)-4-(1-(2-methyl-5-nitrobenzamido)ethyl)piperidine-1-carboxylate (380A-2)

To a solution of tert-butyl (R)-4-(1-aminoethyl)piperidine-1-carboxylate (500 mg, 2.19 mmol, 1.0 eq) and 2-methyl-5-nitrobenzoic acid (397 mg, 2.19 mmol, 0.53 mL, 1.0 eq) in DCM (10 mL) were added TEA (444 mg, 4.38 mmol, 610 μL, 2.0 eq), EDCI (504 mg, 2.63 mmol, 1.2 eq) and HOBt (355 mg, 2.63 mmol, 1.2 eq). The mixture was stirred at 25° C. for 1 h. LCMS indicated that the starting material was completely consumed, and the desired mass was detected. The reaction mixture was poured into H₂O (15 mL) and extracted with DCM (5.0 mL×3). The combined organic layers were dried over Na₂SO₄, filtered, and concentrated under vacuum to give the crude tert-butyl (R)-4-(1-(2-methyl-5-nitrobenzamido)ethyl)piperidine-1-carboxylate (950 mg) as a colorless oil. M−56+H⁺=336.3 (LCMS).

### Step 2: (R)-2-Methyl-5-nitro-N-(1-(piperidin-4-yl)ethyl)benzamide (380A-3)

To a stirred solution of tert-butyl (R)-4-(1-(2-methyl-5-nitrobenzamido)ethyl)piperidine-1-carboxylate (800 mg, 2.04 mmol, 1.0 eq) in EtOAc (10 mL) was added HCl/EtOAc (4 M, 2.0 mL). The mixture was stirred at 20° C. for 1 h. LCMS indicated that the starting material was completely consumed, and the desired mass was detected. The mixture was concentrated under vacuum at 30° C. to give the crude (R)-2-methyl-5-nitro-N-(1-(piperidin-4-yl)ethyl)benzamide (800 mg) as a yellow oil, which was used in the next step without any further purification. M+H⁺=292.1 (LCMS).

### Step 3: (R)—N-(1-(1-(Ethylsulfonyl) piperidin-4-yl)ethyl)-2-methyl-5-nitrobenzamide (380A-4)

To a solution of (R)-2-methyl-5-nitro-N-(1-(piperidin-4-yl)ethyl)benzamide (500 mg, 1.72 mmol, 1.0 eq) in DCM (30 mL) were added TEA (521 mg, 5.15 mmol, 717 μL, 3.0 eq) and ethanesulfonyl chloride (265 mg, 2.06 mmol, 1.2 eq). The mixture was stirred at 0° C. for 1 h. LCMS indicated that the starting material was completely consumed, and the desired mass was detected. The reaction mixture was poured into H₂O (30 mL) and extracted with DCM (10 mL×3). The combined organic layers were dried over Na₂SO₄, filtered, and concentrated under vacuum to give a residue, which was purified by flash silica gel chromatography using a gradient of EtOAc/petroleum ether from 1/100 to 1/1. (R)—N-(1-(1-(Ethylsulfonyl) piperidin-4-yl)ethyl)-2-methyl-5-nitrobenzamide (500 mg, 1.30 mmol, 76% yield) was obtained as a colorless oil. M+H⁺=384.1 (LCMS).

### Step 4: (R)-5-Amino-N-(1-(1-(ethylsulfonyl) piperidin-4-yl)ethyl)-2-methylbenzamide (380A-5)

To a solution of (R)—N-(1-(1-(ethylsulfonyl) piperidin-4-yl)ethyl)-2-methyl-5-nitrobenzamide (500 mg, 1.30 mmol, 1.0 eq) in a mixture of MeOH (10 mL) and H₂O (2.5 mL) were added iron powder (365 mg, 6.52 mmol, 5.0 eq) and NH₄Cl (349 mg, 6.52 mmol, 5.0 eq). The mixture was stirred at 80° C. for 16 h. LCMS indicated that the starting material was completely consumed, and the desired mass was detected. The reaction mixture was allowed to cool to room temperature, poured into H₂O (20 mL) and extracted with EtOAc (5.0 mL×3). The combined organic layers were dried over Na₂SO₄, filtered, and concentrated under vacuum to give the crude (R)-5-amino-N-(1-(1-(ethylsulfonyl) piperidin-4-yl)ethyl)-2-methylbenzamide (26.8 mg, 83.0 μmol, 29% yield) as a white solid. M+H⁺=354.2 (LCMS).

### Step 5: tert-Butyl (R)-3-((3-((1-(1-(ethylsulfonyl) piperidin-4-yl)ethyl)carbamoyl)-4-methylphenyl)amino)azetidine-1-carboxylate (Compound 161)

To a solution of (R)-5-amino-N-(1-(1-(ethylsulfonyl) piperidin-4-yl)ethyl)-2-methylbenzamide (50.0 mg, 141 μmol, 1.0 eq) and tert-butyl 3-oxoazetidine-1-carboxylate (24.3 mg, 141 μmol, 1.0 eq) in MeOH (4.0 mL) was added NaBH₃CN (10.7 mg, 170 μmol, 1.2 eq). The resulting mixture was stirred at 25° C. for 12 h. LCMS indicated that the starting material was completely consumed, and the desired mass was detected. The reaction mixture was poured into H₂O (5.0 mL) and extracted with EtOAc (3.0 mL×3). The combined organic layers were dried over Na₂SO₄, filtered, and concentrated under vacuum to give a residue which was purified by preparative HPLC (Phenomenex Luna C18 column (70×30 mm, 3 μm); flow rate: 25 mL/min; gradient: 30%-70% B over 8 min; mobile phase A: 0.2% aqueous FA, mobile phase B: acetonitrile) to give tert-butyl (R)-3-((3-((1-(1-(ethylsulfonyl) piperidin-4-yl)ethyl)carbamoyl)-4-methylphenyl)amino)azetidine-1-carboxylate (2.09 mg, 4.11 μmol, 3% yield) as a white solid. M+Na⁺=531.2 (LCMS); ¹H NMR (400 MHZ, CD₃OD) δ 7.04 (d, J=8.4 Hz, 1H), 6.53 (d, J=2.5 Hz, 1H), 6.48 (dd, J=2.4, 8.2 Hz, 1H), 5.49 (br d, J=8.9 Hz, 1H), 4.34-4.25 (m, 2H), 4.24-4.14 (m, 2H), 3.92-3.84 (m, 2H), 3.72 (dd, J=4.4, 8.9 Hz, 2H), 2.96 (q, J=7.5 Hz, 2H), 2.85-2.73 (m, 2H), 2.31 (s, 3H), 1.89-1.76 (m, 2H), 1.51-1.40 (m, 13H), 1.37 (t, J=7.4 Hz, 3H), 1.22 (d, J=6.8 Hz, 3H).

### Example 381: (R)-5-(Azetidin-3-ylamino)-N-(1-(1-(ethylsulfonyl) piperidin-4-yl)ethyl)-2-methylbenzamide (Compound 166)

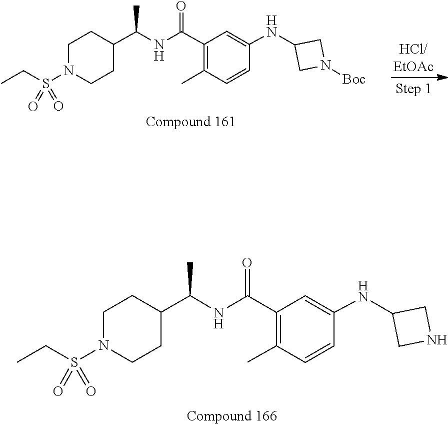 [SMILES: CCS(=O)(=O)N1CCC([C@@H](C)NC(=O)c2cc(NC3CN(C(=O)OC(C)(C)C)C3)ccc2C)CC1 ; CCS(=O)(=O)N1CCC([C@@H](C)NC(=O)c2cc(NC3CNC3)ccc2C)CC1]

### Step 1: (R)-5-(Azetidin-3-ylamino)-N-(1-(1-(ethylsulfonyl) piperidin-4-yl)ethyl)-2-methylbenzamide (Compound 166)

To a stirred solution of tert-butyl (R)-3-((3-((1-(1-(ethylsulfonyl) piperidin-4-yl)ethyl) carbamoyl)-4-methylphenyl)amino)azetidine-1-carboxylate (150 mg, 295 μmol, 1.0 eq) in EtOAc (5.0 mL) was added HCl/EtOAc (4 M, 300 μL). The mixture was stirred at 25° C. for 30 min. LCMS indicated that the starting material was completely consumed, and the desired mass was detected. The mixture was concentrated under vacuum at 25° C. to give a residue which was treated with small amount of NH₃·H₂O to pH 7 and purified by preparative HPLC (Waters Xbridge BEH C18 column (100×30 mm, 10 μm); flow rate: 25 mL/min; gradient: 15%-45% B over 8 min; mobile phase A: 10 mM aqueous NH₄HCO₃, mobile phase B: acetonitrile). (R)-5-(Azetidin-3-ylamino)-N-(1-(1-(ethylsulfonyl) piperidin-4-yl)ethyl)-2-methylbenzamide (30.0 mg, 71.6 μmol, 24% yield) was obtained as a white solid. M+H⁺=409.1 (LCMS); ¹H NMR (400 MHZ, CD₃OD) δ 7.00 (d, J=8.1 Hz, 1H), 6.60-6.49 (m, 2H), 4.38 (t, J=7.1 Hz, 1H), 4.05-3.92 (m, 2H), 4.12-3.90 (m, 1H), 3.80 (br d, J=12.5 Hz, 2H), 3.61 (br t, J=8.1 Hz, 2H), 3.03 (q, J=7.3 Hz, 2H), 2.89-2.74 (m, 2H), 2.24 (s, 3H), 1.95-1.80 (m, 2H), 1.59 (dt, J=3.2, 7.4 Hz, 1H), 1.47-1.27 (m, 5H), 1.21 (d, J=6.8 Hz, 3H).

### Example 382: N-(2-Methoxy-1-(naphthalen-1-yl)ethyl)-2-methyl-5-nitrobenzamide (Compound 108)

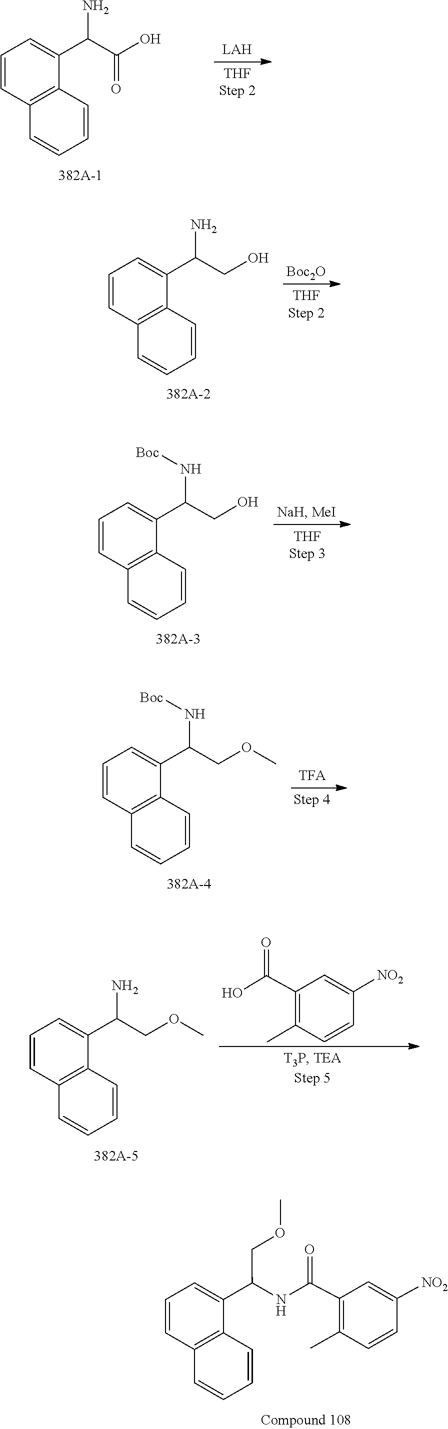

### Step 1: 2-Amino-2-(naphthalen-1-yl)ethanol (382A-2)

To a solution of 2-amino-2-(naphthalen-1-yl) acetic acid (400 mg, 1.99 mmol, 1.0 eq) in THF (32 mL) was added LiAlH₄ (755 mg, 19.9 mmol, 10 eq) in portions at 0° C. The mixture was stirred at 65° C. for 2 h. LCMS indicated that the starting material was completely consumed, and the desired mass was detected. The reaction mixture was cooled to 0° C., quenched by addition of 0.8 mL of H₂O, followed by 0.8 mL of 10% aqueous NaOH. The suspension was stirred at room temperature for 5 min, then filtered through a pad of celite. The combined filtrates were dried over Na₂SO₄, filtered, and concentrated under vacuum to give the crude 2-amino-2-(naphthalen-1-yl)ethanol (400 mg) as an orange solid, which was used in the next step without any further purification. M+H⁺=188.3 (LCMS).

### Step 2: tert-Butyl(2-hydroxy-1-(naphthalen-1-yl)ethyl)carbamate (382A-3)

To a solution of 2-amino-2-(naphthalen-1-yl)ethanol (200 mg, 1.07 mmol, 1.0 eq) in THF (1.0 mL) was added Boc₂O (233 mg, 1.07 mmol, 246 μL, 1.0 eq). The resulting mixture was stirred at 20° C. for 13 h. LCMS indicated that the starting material was completely consumed, and the desired mass was detected. The mixture was concentrated under vacuum to give a residue which was triturated with EtOAc (2.0 mL) and filtered. The solid cake was dried in vacuum to give tert-butyl(2-hydroxy-1-(naphthalen-1-yl)ethyl)carbamate (300 mg, 1.04 mmol, 98% yield) as a white solid. M+H⁺=288.3 (LCMS).

### Step 3: tert-Butyl(2-methoxy-1-(naphthalen-1-yl)ethyl)carbamate (382A-4)

To a solution of tert-butyl(2-hydroxy-1-(naphthalen-1-yl)ethyl)carbamate (100 mg, 348 μmol, 1.0 eq) in THF (1.0 mL) was added sodium hydride (16.7 mg, 418 μmol, 60% purity, 1.2 eq) and Mel (54.3 mg, 383 μmol, 23.8 μL, 1.1 eq) at 0° C. The mixture was stirred at 20° C. for 2 h. LCMS indicated that the starting material was completely consumed, and the desired mass was detected. The mixture was poured into H₂O (10 mL) and extracted with EtOAc (10 mL×3). The combined organic layers were dried over Na₂SO₄, filtered, and concentrated under vacuum to give a residue which was purified by preparative TLC (petroleum ether/EtOAc=3/1, R_{f}=0.6). tert-Butyl(2-methoxy-1-(naphthalen-1-yl)ethyl)carbamate (60.0 mg, 199 μmol, 57% yield) was obtained as a white solid. M+H⁺=302.3 (LCMS).

### Step 4: 2-Methoxy-1-(naphthalen-1-yl) ethan-1-amine (382A-5)

To a solution of tert-butyl(2-methoxy-1-(naphthalen-1-yl)ethyl)carbamate (50.0 mg, 166 μmol, 1.0 eq) in DCM (1.0 mL) was added TFA (333 μL). The mixture was stirred at 20° C. for 2 h. LCMS indicated that the starting material was completely consumed, and the desired mass was detected. The mixture was concentrated under vacuum to give the crude 2-methoxy-1-(naphthalen-1-yl) ethan-1-amine (30.0 mg, TFA salt) as a yellow oil, which was used in the next step without any further purification. M+H⁺=202.2 (LCMS).

### Step 5: N-(2-Methoxy-1-(naphthalen-1-yl)ethyl)-2-methyl-5-nitrobenzamide (Compound 108)

To a solution of 2-methyl-5-nitrobenzoic acid (29.7 mg, 164 μmol, 1.1 eq) and 2-methoxy-1-(naphthalen-1-yl) ethan-1-amine (30.0 mg, 149 μmol, 1.0 eq, TFA salt) in DCM (2.0 mL) was added TEA (30.2 mg, 298 μmol, 41.5 μL, 2.0 eq) and T₃P (94.9 mg, 149 μmol, δ8.7 μL, 50% purity in EtOAc, 1.0 eq). The mixture was stirred at 20° C. for 3 h. LCMS indicated that the starting material was completely consumed, and the desired mass was detected. The mixture was poured into H₂O (5.0 mL) and extracted with DCM (5.0 mL×3). The combined organic layers were dried over Na₂SO₄, filtered, and concentrated under vacuum to give a residue which was purified by preparative HPLC (Waters Xbridge BEH C18 column (100×30 mm, 10 μm);

flow rate: 25 mL/min; gradient: 40%-70% B over 8 min; mobile phase A: 10 mM aqueous NH₄HCO₃, mobile phase B: acetonitrile). N-(2-Methoxy-1-(naphthalen-1-yl)ethyl)-2-methyl-5-nitrobenzamide (10.0 mg, 27.1 μmol, 18% yield) was obtained as an off-white solid. M+H⁺=365.1 (LCMS); ¹H NMR (400 MHZ, CDCl₃) δ 8.28 (d, J=2.3 Hz, 1H), 8.22 (d, J=8.5 Hz, 1H), 8.17 (dd, J=2.4, 8.4 Hz, 1H), 7.91 (d, J=8.3 Hz, 1H), 7.85 (d, J=8.1 Hz, 1H), 7.65-7.58 (m, 2H), 7.57-7.46 (m, 2H), 7.40 (d, J=8.5 Hz, 1H), 6.52 (br d, J=7.6 Hz, 1H), 6.31-6.10 (m, 1H), 4.07-3.85 (m, 2H), 3.46 (s, 3H), 2.55 (s, 3H).

### Example 383: 5-Amino-N-(2-methoxy-1-(naphthalen-1-yl)ethyl)-2-methylbenzamide (Compound 118)

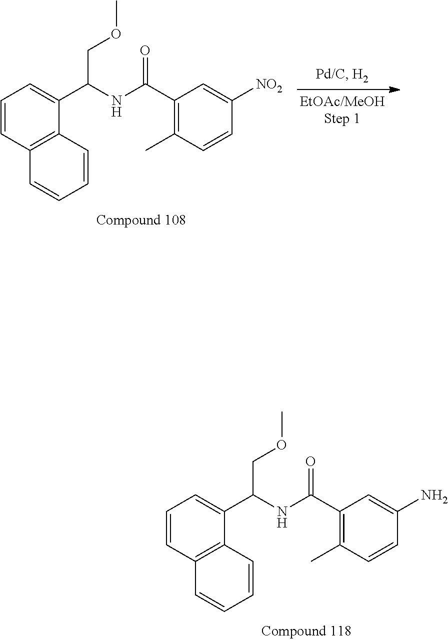 [SMILES: COCC(NC(=O)c1cc([N+](=O)[O-])ccc1C)c1cccc2ccccc12 ; COCC(NC(=O)c1cc(N)ccc1C)c1cccc2ccccc12]

### Step 1: 5-Amino-N-(2-methoxy-1-(naphthalen-1-yl)ethyl)-2-methylbenzamide (Compound 118)

To a solution of N-(2-methoxy-1-(naphthalen-1-yl)ethyl)-2-methyl-5-nitrobenzamide (50.0 mg, 137 μmol, 1.0 eq) in EtOAc (5.0 mL) was added 10% palladium on carbon (50.0 mg) under a N₂ atmosphere. The suspension was degassed and purged with H₂ three times. The mixture was stirred at 20° C. for 3 h under a H₂ (15 psi) atmosphere. LCMS indicated that the starting material completely consumed, and the desired product was detected. The suspension was filtered through a pad of Celite and the filter cake was washed with EtOAc (2.0 mL×3). The combined filtrates were concentrated under vacuum to give a residue which was purified by preparative HPLC (Waters Xbridge BEH C18 column (100×30 mm, 10 μm); flow rate: 25 mL/min; gradient: 30%-60% B over 8 min; mobile phase A: 10 mM aqueous NH₄HCO₃, mobile phase B: acetonitrile). 5-Amino-N-(2-methoxy-1-(naphthalen-1-yl)ethyl)-2-methylbenzamide (13.1 mg, 37.0 μmol, 27% yield) was obtained as a white solid. M+H⁺=335.2 (LCMS); ¹H NMR (400 MHZ, CDCl₃) δ 8.24-8.16 (m, 1H), 7.92-7.84 (m, 1H), 7.79 (d, J=8.3 Hz, 1H), 7.59-7.37 (m, 4H), 6.94 (d, J=8.3 Hz, 1H), 6.85 (br d, J=1.6 Hz, 1H), 6.74 (br d, J=8.0 Hz, 1H), 6.70 (br s, 1H), 6.21-6.12 (m, 1H), 3.94-3.79 (m, 2H), 3.38 (s, 3H), 2.28 (s, 3H).

### Example 384: 2-Methyl-N-(naphthalen-1-yl(tetrahydro-2H-pyran-4-yl)methyl)-5-nitrobenzamide (Compound 105)

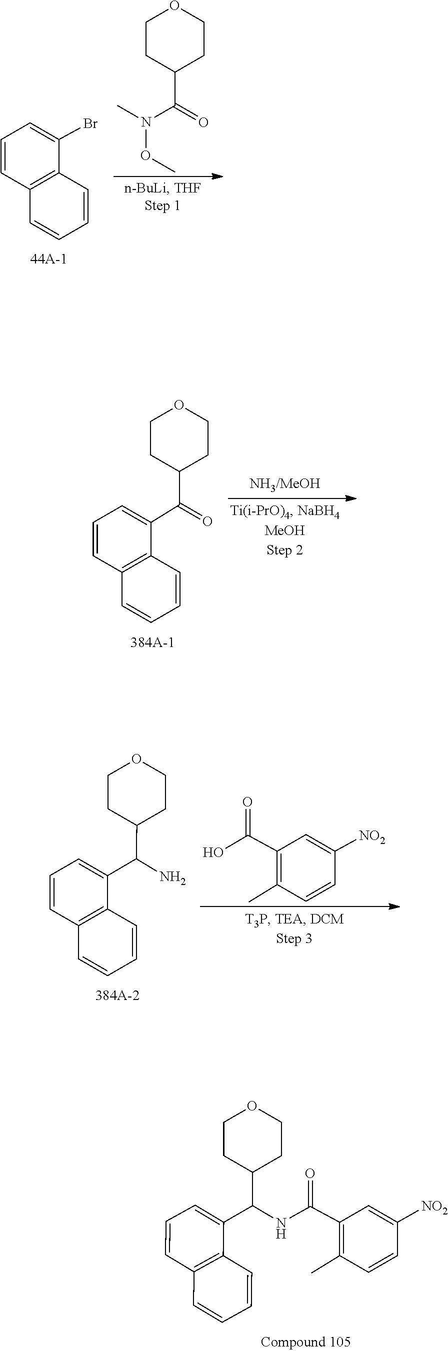

### Step 1: Naphthalen-1-yl(tetrahydro-2H-pyran-4-yl)methanone (384A-1)

To a solution of 1-bromonaphthalene (430 mg, 2.08 mmol, 289 μL, 1.2 eq) in THF (10 mL) was added n-BuLi (2.5 M in hexane, 831 μL, 1.2 eq) dropwise at −78° C. under a N₂ atmosphere. The mixture was stirred at the same temperature for 50 min. A solution of N-methoxy-N-methyltetrahydro-2H-pyran-4-carboxamide (300 mg, 1.73 mmol, 1.0 eq) in THF (10 mL) was added dropwise at the same temperature. The resulting mixture was stirred at −78° C. for 2 h. LCMS indicated that the starting material was completely consumed, and the desired mass was detected. The reaction mixture was allowed to warm to room temperature, poured into H₂O (10 mL) and extracted with EtOAc (2.0 mL×5). The combined organic layers were dried over Na₂SO₄, filtered, and concentrated under vacuum to give a residue which was purified by flash silica gel chromatography using a gradient of EtOAc/petroleum ether from 1/100 to 1/10. Naphthalen-1-yl(tetrahydro-2H-pyran-4-yl)methanone (200 mg, 832 μmol, 48% yield) was obtained as a colorless oil. M+H⁺=241.3 (LCMS); ¹H NMR (400 MHZ, CDCl₃) δ 8.29 (d, J=8.2 Hz, 1H), 7.99 (d, J=8.2 Hz, 1H), 7.94-7.86 (m, 1H), 7.75 (d, J=7.1 Hz, 1H), 7.63-7.47 (m, 3H), 4.05 (td, J=3.4, 11.6 Hz, 2H), 3.59-3.38 (m, 3H), 1.98-1.79 (m, 4H).

### Step 2: Naphthalen-1-yl(tetrahydro-2H-pyran-4-yl)methanamine (384A-2)

To a solution of naphthalen-1-yl(tetrahydro-2H-pyran-4-yl)methanone (150 mg, 624 μmol, 1.0 eq) in MeOH (2.0 mL) were added NH₃/MeOH (20 M, 5.0 mL) and Ti(i-PrO)₄ (δ87 mg, 3.12 mmol, 921 μL, 5.0 eq) at 0° C. The mixture was stirred at 20° C. for 12 h. After that, the mixture was cooled to 0° C. and NaBH₄ (118 mg, 3.12 mmol, 5.0 eq) was added. The resulting mixture was stirred at 0° C. for 1 h, and then stirred at 20° C. for another 1 h. LCMS indicated that the starting material was completely consumed, and the desired mass was detected. The reaction mixture was poured into H₂O (5.0 mL) and extracted with EtOAc (1.0 mL×5). The combined organic layers were dried over Na₂SO₄, filtered, and concentrated under vacuum to give the crude naphthalen-1-yl(tetrahydro-2H-pyran-4-yl)methanamine (150 mg) as a brown oil, which was used in the next step without any further purification. M−17+H⁺=225.3 (LCMS).

### Step 3: 2-Methyl-N-(naphthalen-1-yl(tetrahydro-2H-pyran-4-yl)methyl)-5-nitrobenzamide (Compound 105)

To a solution of naphthalen-1-yl(tetrahydro-2H-pyran-4-yl)methanamine (100 mg, 414 μmol, 1.0 eq) and 2-methyl-5-nitro-benzoic acid (75.1 mg, 414 μmol, 1.0 eq) in DCM (5.0 mL) were added TEA (126 mg, 1.24 mmol, 173 μL, 3.0 eq) and T₃P (527 mg, 829 μmol, 493 μL, 50% in EtOAc, 2.0 eq) at 0° C. The mixture was stirred at 20° C. for 16 h. LCMS indicated that the starting material was completely consumed, and the desired mass was detected. The mixture was poured into H₂O (6.0 mL) and extracted with DCM (2.0 mL×3). The combined organic layers were dried over Na₂SO₄, filtered, and concentrated under vacuum to give a residue which was purified by preparative HPLC (Phenomenex Luna C18 column (150×30 mm, 5 μm); flow rate: 25 mL/min; gradient: 40%-75% B over 8 min; mobile phase A: 0.2% aqueous FA, mobile phase B: acetonitrile). 2-Methyl-N-(naphthalen-1-yl(tetrahydro-2H-pyran-4-yl)methyl)-5-nitrobenzamide (13.7 mg, 32.9 μmol, 8% yield) was obtained as an off-white solid. M+H⁺=405.1 (LCMS); ¹H NMR (400 MHZ, CDCl₃) δ 8.31 (d, J=8.5 Hz, 1H), 8.19-8.09 (m, 2H), 7.92 (d, J=8.1 Hz, 1H), 7.85 (dd, J=3.1, 6.1 Hz, 1H), 7.66-7.59 (m, 1H), 7.59-7.53 (m, 1H), 7.52-7.46 (m, 2H), 7.36 (d, J=8.9 Hz, 1H), 6.23-6.09 (m, 1H), 5.95 (t, J=9.1 Hz, 1H), 4.17-3.86 (m, 2H), 3.50-3.28 (m, 2H), 2.45 (s, 3H), 2.35 (br s, 1H), 1.94-1.69 (m, 2H), 1.59-1.40 (m, 2H).

### Example 385: 5-Amino-2-methyl-N-(naphthalen-1-yl(tetrahydro-2H-pyran-4-yl)methyl)benzamide (Compound 109)

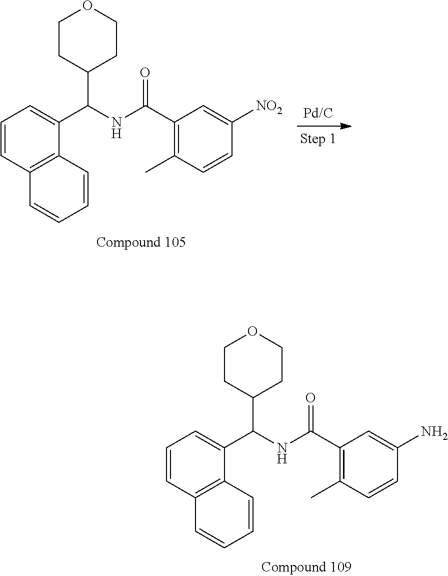 [SMILES: Cc1ccc([N+](=O)[O-])cc1C(=O)NC(c1cccc2ccccc12)C1CCOCC1 ; Cc1ccc(N)cc1C(=O)NC(c1cccc2ccccc12)C1CCOCC1]

### Step 1: 5-Amino-2-methyl-N-(naphthalen-1-yl(tetrahydro-2H-pyran-4-yl)methyl)benzamide (Compound 109)

To a solution of 2-methyl-N-(naphthalen-1-yl(tetrahydro-2H-pyran-4-yl)methyl)-5-nitrobenzamide (80.0 mg, 198 μmol, 1.0 eq) in EtOAc (5.0 mL) and MeOH (5.0 mL) was added 10% palladium on carbon (80.0 mg) under a N₂ atmosphere. The suspension was degassed and purged with H₂ three times. The mixture was stirred at 20° C. for 5 h under a H₂ (15 psi) atmosphere. LCMS indicated that the starting material was completely consumed, and the desired mass was detected. The suspension was filtered through a pad of Celite and the filter cake was washed with EtOAc (5.0 mL×3). The combined filtrates were concentrated under vacuum to give a residue which was purified by preparative HPLC (Phenomenex Luna C18 column (150×30 mm, 5 μm); flow rate: 25 mL/min; gradient: 25%-50% B over 8 min; mobile phase A: 10 mM aqueous NH₄HCO₃, mobile phase B: acetonitrile). 5-Amino-2-methyl-N-(naphthalen-1-yl(tetrahydro-2H-pyran-4-yl)methyl)benzamide (25.8 mg, 68.5 μmol, 35% yield) was obtained as a white solid. M+H⁺=375.1 (LCMS); ¹H NMR (400 MHZ, CDCl₃) δ 8.31 (d, J=8.4 Hz, 1H), 7.90 (d, J=8.1 Hz, 1H), 7.82 (d, J=7.8 Hz, 1H), 7.63-7.41 (m, 4H), 6.96 (d, J=8.8 Hz, 1H), 6.68-6.59 (m, 2H), 6.14-5.87 (m, 2H), 4.07 (br dd, J=2.4, 11.7 Hz, 1H), 3.92 (br d, J=11.0 Hz, 1H), 3.58 (br s, 2H), 3.44-3.28 (m, 2H), 2.39-2.14 (m, 4H), 1.88-1.67 (m, 2H), 1.62-1.44 (m, 2H).

### Example 386: 5-Amino-2-methyl-N-(naphthalen-1-ylsulfonyl)benzamide (Compound 242)

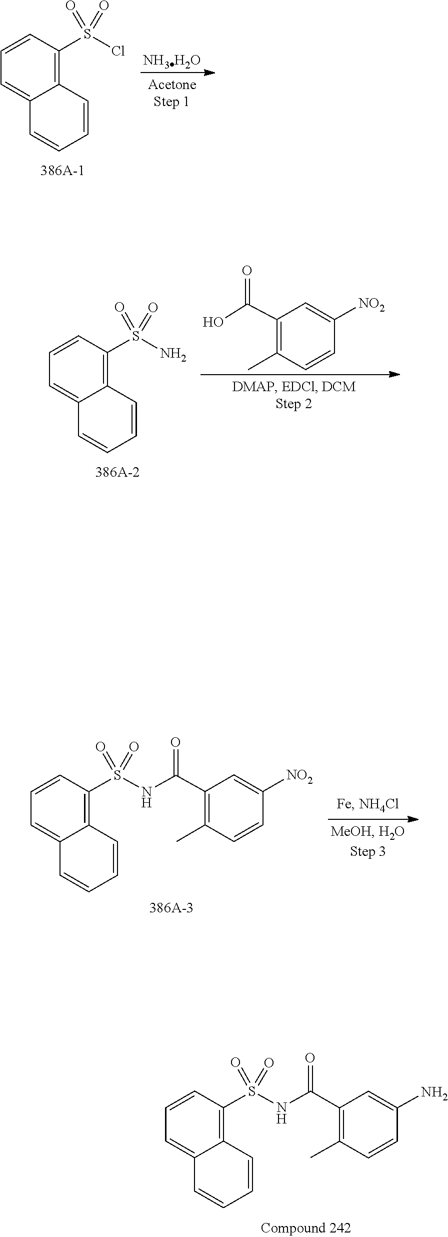

### Step 1: Naphthalene-1-sulfonamide (386A-2)

To a solution of naphthalene-1-sulfonyl chloride (500 mg, 2.21 mmol, 1.0 eq) in acetone (10 mL) was added NH₃·H₂O (12 mL, 25% purity) at 0° C. The mixture was stirred at 0° C. for 15 min. LCMS indicated that the starting material was completely consumed, and the desired mass was detected. The mixture was poured into H₂O (10 ml) and extracted with EtOAc (5.0 mL×3). The combined organic layers were dried over Na₂SO₄, filtered, and concentrated under vacuum to give the crude naphthalene-1-sulfonamide (400 mg, 1.93 mmol, δ8% yield) as a white solid. M+H⁺=208.0 (LCMS); ¹H NMR (400 MHZ, DMSO-d₆) δ 8.63 (d, J=8.4 Hz, 1H), 8.19 (d, J=8.3 Hz, 1H), 8.13 (d, J=7.3 Hz, 1H), 8.08 (d, J=7.7 Hz, 1H), 7.73-7.60 (m, 5H).

### Step 2: 2-Methyl-N-(naphthalen-1-ylsulfonyl)-5-nitrobenzamide (386A-3)

To a solution of naphthalene-1-sulfonamide (100 mg, 483 μmol, 1.0 eq) and 2-methyl-5-nitro-benzoic acid (87.4 mg, 483 μmol, 1.0 eq) in DCM (4.0 mL) were added DMAP (δ8.4 mg, 724 μmol, 1.5 eq) and EDCI (185 mg, 965 μmol, 2.0 eq). The mixture was stirred at 25° C. for 20 h. LCMS indicated that the starting material was completely consumed, and the desired mass was detected. The reaction mixture was poured into H₂O (4.0 mL) and extracted with EtOAc (5.0 mL×3). The combined organic layers were dried over Na₂SO₄ filtered, and concentrated under vacuum to give the crude product 2-methyl-N-(naphthalen-1-ylsulfonyl)-5-nitrobenzamide (170 mg, 459 μmol, 95% yield) as a white solid. M+H⁺=371.2 (LCMS).

### Step 3: 5-Amino-2-methyl-N-(naphthalen-1-ylsulfonyl)benzamide (Compound 242)

To a solution of 2-methyl-N-(naphthalen-1-ylsulfonyl)-5-nitrobenzamide (100 mg, 270 μmol, 1.0 eq) in a mixture of MeOH (10 mL) and H₂O (2.5 mL) was added iron powder (75.4 mg, 1.35 mmol, 5.0 eq), followed by NH₄Cl (72.2 mg, 1.35 mmol, 5.0 eq). The mixture was stirred at 80° C. for 12 h. LCMS indicated that the starting material was completely consumed, and the desired mass was detected. The mixture was poured into H₂O (10 mL) and extracted with EtOAc (5.0 mL×3). The combined organic layers were dried over Na₂SO₄, filtered, and concentrated under vacuum to give a residue which was purified by preparative HPLC (Phenomenex Gemini C18 column (75×30 mm, 3 μm); flow rate: 25 mL/min; gradient: 20%-40% B over 8 min; mobile phase A: 0.04% aqueous HCl, mobile phase B: acetonitrile). 5-Amino-2-methyl-N-(naphthalen-1-ylsulfonyl)benzamide (16.0 mg, 41.4 μmol, 15% yield, HCl salt) was obtained as a white solid. M+H⁺=341.1 (LCMS); ¹H NMR (400 MHZ, DMSO-d₆) δ 8.63 (d, J=8.4 Hz, 1H), 8.37 (dd, J=7.5, 14.9 Hz, 2H), 8.16 (d, J=7.9 Hz, 1H), 7.81-7.66 (m, 3H), 7.24-6.96 (m, 3H), 1.85 (s, 3H).

### Example 387: (R)-5-Amino-2-methyl-N-(1-(naphthalen-1-yl)ethyl)benzenesulfonamide (Compound 138)

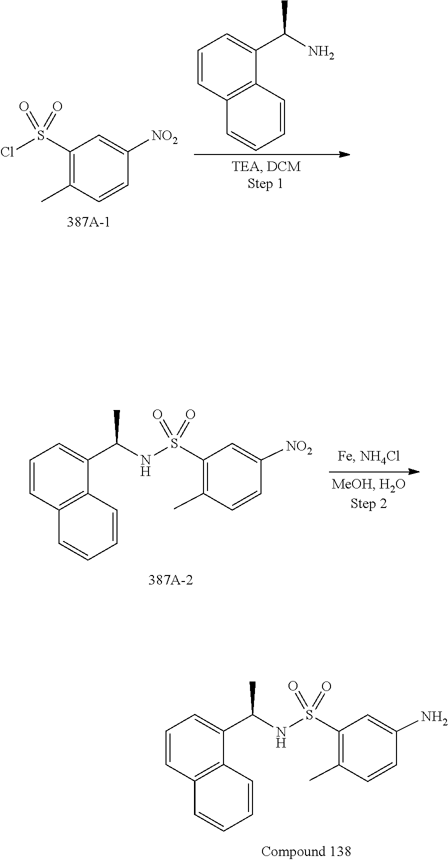 [SMILES: Cc1ccc([N+](=O)[O-])cc1S(=O)(=O)Cl ; C[C@@H](N)c1cccc2ccccc12 ; Cc1ccc([N+](=O)[O-])cc1S(=O)(=O)N[C@H](C)c1cccc2ccccc12 ; Cc1ccc(N)cc1S(=O)(=O)N[C@H](C)c1cccc2ccccc12]

### Step 1: (R)-2-Methyl-N-(1-(naphthalen-1-yl)ethyl)-5-nitrobenzenesulfonamide (387A-2)

To a solution of 2-methyl-5-nitrobenzenesulfonyl chloride (300 mg, 1.27 mmol, 1.0 eq) and (R)-1-(naphthalen-1-yl) ethan-1-amine (218 mg, 1.27 mmol, 204 μL, 1.0 eq) in DCM (5.0 mL) was added TEA (155 mg, 1.53 mmol, 213 μL, 1.2 eq). The mixture was stirred at 20° C. for 16 h. LCMS indicated that the starting material was completely consumed, and the desired mass was detected. The mixture was poured into H₂O (5.0 mL) and extracted with DCM (2.0 mL×3). The combined organic layers were dried over Na₂SO₄, filtered, and concentrated under vacuum to give (R)-2-methyl-N-(1-(naphthalen-1-yl)ethyl)-5-nitrobenzenesulfonamide (400 mg) as a pale yellow solid, which was used in the next step without any further purification. (M+18)⁺=388.2 (LCMS).

### Step 2: (R)-5-Amino-2-methyl-N-(1-(naphthalen-1-yl)ethyl)benzenesulfonamide (Compound 138)

To a solution of (R)-2-methyl-N-(1-(naphthalen-1-yl)ethyl)-5-nitrobenzenesulfonamide (200 mg, 540 μmol, 1.0 eq) in MeOH (2.5 mL) and H₂O (0.5 mL) were added iron powder (151 mg, 2.70 mmol, 5.0 eq) and NH₄Cl (144 mg, 2.70 mmol, 5.0 eq). The mixture was stirred at 80° C. for 2 h. LCMS indicated that the starting material was completely consumed, and the desired mass was detected. The mixture was poured into H₂O (6.0 mL) and extracted with EtOAc (2.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 20%-50% B over 8 min; mobile phase A: 0.1% aqueous TFA, mobile phase B: acetonitrile). (R)-5-Amino-2-methyl-N-(1-(naphthalen-1-yl)ethyl)benzenesulfonamide (93.8 mg, 200 μmol, 37% yield, TFA salt) was obtained as a yellow solid. M+H⁺=341.0 (LCMS); ¹H NMR (400 MHZ, CDCl₃) δ 7.97-7.89 (m, 1H), 7.79-7.73 (m, 1H), 7.65 (d, J=8.1 Hz, 1H), 7.58 (d, J=1.6 Hz, 1H), 7.47 (d, J=7.3 Hz, 1H), 7.44-7.39 (m, 2H), 7.34-7.28 (m, 1H), 7.13-7.07 (m, 2H), 5.25 (q, J=6.9 Hz, 1H), 2.56-2.30 (m, 3H), 1.57 (d, J=6.9 Hz, 3H).

### Example 388: 5-Amino-2-methyl-N-(1-(naphthalen-1-yl)cyclopropyl)-4-(phenylethynyl)benzamide (Compound 217)

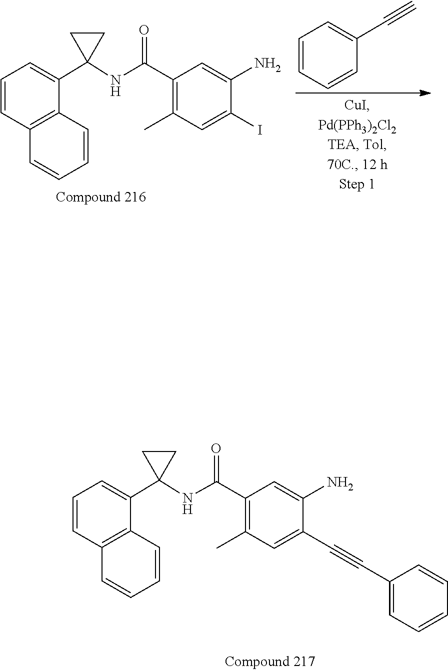

### Step 1: 5-Amino-2-methyl-N-(1-(naphthalen-1-yl)cyclopropyl)-4-(phenylethynyl)benzamide (Compound 217)

A mixture of 5-amino-4-iodo-2-methyl-N-(1-(naphthalen-1-yl)cyclopropyl)benzamide (100 mg, 226 μmol, 1.0 eq), TEA (45.8 mg, 452 μmol, 62.9 μL, 2.0 eq), Pd(PPh₃)₂Cl₂ (4.76 mg, 6.78 μmol, 0.03 eq), CuI (431 μg, 2.26 μmol, 0.01 eq) and ethynylbenzene (34.6 mg, 339 μmol, 37.2 μL, 1.5 eq) in toluene (5.0 mL) and H₂O (2.5 mL) was degassed and purged with N₂ three times. The resulting mixture was stirred at 70° C. for 12 h under a N₂ atmosphere. LCMS indicated that the starting material was completely consumed, and the desired mass was detected. The reaction mixture was allowed to cool to room temperature, poured into H₂O (10 mL) and extracted with EtOAc (10 mL×3). The combined organic layers were dried over Na₂SO₄, filtered, and concentrated under vacuum to give a residue which was purified by preparative HPLC (Phenomenex luna C18 column (80× 40 mm, 3 μm); flow rate: 25 mL/min; gradient: 43%-83% B over 7 min; mobile phase A: 0.04% aqueous HCl, mobile phase B: acetonitrile). 5-Amino-2-methyl-N-(1-(naphthalen-1-yl)cyclopropyl)-4-(phenylethynyl)benzamide (50.0 mg, 110 μmol, 48% yield, HCl salt) was obtained as a white solid. M+H⁺=417.1 (LCMS); ¹H NMR (400 MHZ, CDCl₃) δ 8.47 (d, J=8.5 Hz, 1H), 7.98-7.88 (m, 2H), 7.81 (d, J=8.3 Hz, 1H), 7.62-7.47 (m, 5H), 7.37-7.31 (m, 3H), 7.11 (s, 1H), 6.51 (s, 1H), 6.47 (s, 1H), 2.08 (s, 3H), 1.57 (br d, J=1.8 Hz, 2H), 1.42-1.38 (m, 2H).

### Example 389: (S)—N-(1-(7-Chloroquinolin-5-yl)cyclopropyl)-2-methyl-5-((1-methylazetidin-2-yl)methoxy)benzamide (Compound 624)

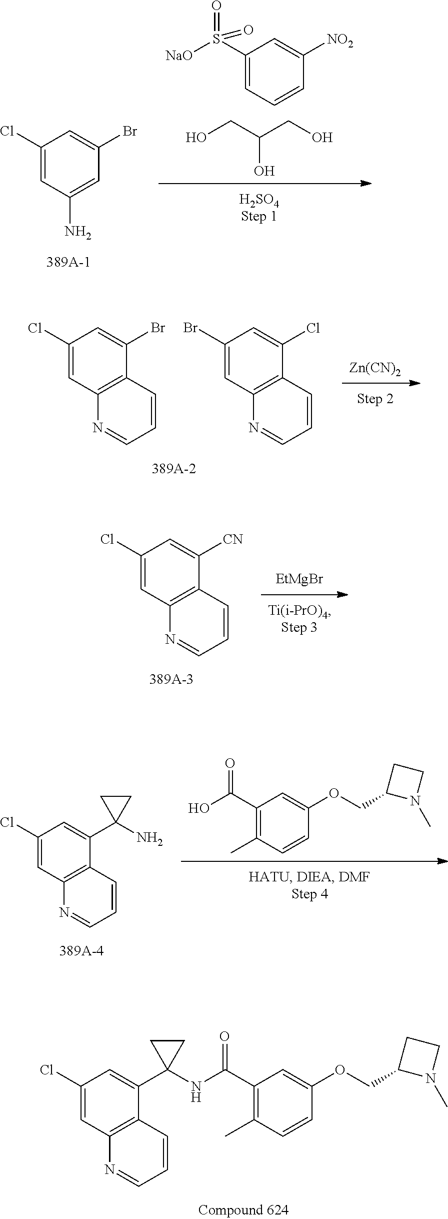

### Step 1: 5-Bromo-7-chloroquinoline (389A-2)

To a solution of 3-bromo-5-chloroaniline (10.0 g, 48.4 mmol, 1.0 eq) and sodium 3-nitrobenzenesulfonate (21.8 g, 96.9 mmol, 2.0 eq) in H₂SO₄ (125 g, 1.25 mol, 68.1 mL, 98% purity, 26 eq) and H₂O (29 mL) was added glycerol (17.8 g, 194 mmol, 14.5 mL, 4.0 eq) at 100° C. The mixture was stirred at 125° C. for 4 h. LCMS indicated that the starting material was completely consumed, and the desired mass was detected. The mixture was treated with water (20 mL) and extracted with EtOAc (20 mL×3). The combined organic layers were dried over Na₂SO₄, filtered, and concentrated under vacuum to give a residue which was purified by silica gel chromatography using a gradient of EtOAc/petroleum ether from 1/20 to 1/5. A mixture of 7-bromo-5-chloroquinoline and 5-bromo-7-chloroquinoline (8.00 g, 33.0 mmol, 64% yield) was obtained as a white solid, which was hard to be separated from each other. M+H⁺=241.9 (LCMS).

### Step 2: 7-Chloroquinoline-5-carbonitrile (389A-3)

To a solution of 7-bromo-5-chloroquinoline and 5-bromo-7-chloroquinoline (1.00 g, 4.12 mmol, 1.0 eq) in DMF (10 mL) were added Zn(CN)₂ (242 mg, 2.06 mmol, 131 μL, 0.5 eq) and Pd(PPh₃)₄ (477 mg, 412 μmol, 0.1 eq). The mixture was stirred at 120° C. for 16 h. LCMS indicated that the starting material was completely consumed, and the desired mass was detected. The mixture was treated with water (10 mL) and extracted with EtOAc (10 mL×3). The combined organic layers were dried over Na₂SO₄ filtered, and concentrated under vacuum to give a residue which was purified by flash silica gel chromatography using a gradient of EtOAc/petroleum ether from 0/1 to 1/10. 7-Chloroquinoline-5-carbonitrile (150 mg, 795 μmol, 19% yield) was obtained as a white solid. M+H⁺=189.0 (LCMS).

### Step 3: 1-(7-Chloroquinolin-5-yl)cyclopropan-1-amine (389A-4)

A mixture of 7-chloroquinoline-5-carbonitrile (1.30 g, 6.89 mmol, 1.0 eq) in anhydrous Et₂O (100 mL) was degassed and purged with N₂ three times. The mixture was stirred with a mechanical stirrer at −78° C. To this mixture was added Ti(i-PrO)₄ (2.94 g, 10.3 mmol, 3.05 mL, 1.5 eq) slowly, and then EtMgBr (2 M in Et₂O, 7.58 mL, 2.2 eq) was added dropwise to maintain the temperature between −78° C. and −73° C. over 1 h under a N₂ atmosphere. After the addition was complete, the resulting mixture was stirred at the same temperature for 10 min and warmed to 20° C. over 1 h. BF₃·Et₂O (1.96 g, 13.8 mmol, 1.70 mL, 2.0 eq) was added slowly with no obvious temperature change. The mixture was stirred at 20° C. for another 1 h. LCMS indicated that the starting material was completely consumed, and the desired mass was detected. The reaction mixture was added into a mixture of HCl (1 M aqueous) (100 mL) and extracted with MTBE (50 mL×2). The aqueous layer was basified to pH 8 by using NaOH (2 M aqueous), and a precipitate was formed. The mixture was filtered through a pad of Celite and the slurry was washed with DCM several times. The combined filtrate was extracted with DCM (50 mL×4). The combined organic layers were dried over Na₂SO₄, filtered, and concentrated under vacuum to give a residue which was purified by preparative HPLC (Phenomenex Luna C18 column (100×30 mm, 5 μm); flow rate: 25 mL/min; gradient: 5%-25% B over 10 min; mobile phase A: 0.04% aqueous HCl, mobile phase B: acetonitrile) to give 1-(7-chloroquinolin-5-yl)cyclopropan-1-amine (4.00 mg, 18.3 μmol, HCl salt) as a white solid. ¹H NMR (400 MHZ, DMSO-d₆) δ 9.04 (dd, J=1.5, 4.1 Hz, 1H), 8.77 (br s, 1H), 8.20 (d, J=1.9 Hz, 1H), 7.87 (d, J=2.1 Hz, 1H), 7.74 (dd, J=4.3, 8.6 Hz, 1H), 1.60 (s, 2H), 1.31 (s, 2H).

### Step 4: (S)—N-(1-(7-Chloroquinolin-5-yl)cyclopropyl)-2-methyl-5-((1-methylazetidin-2-yl)methoxy)benzamide (Compound 624)

To a solution of(S)-2-methyl-5-((1-methylazetidin-2-yl)methoxy)benzoic acid (5.38 mg, 22.9 μmol, 1.0 eq) in DMF (15 mL) was added 1-(7-chloroquinolin-5-yl)cyclopropan-1-amine (5.00 mg, 22.9 μmol, 1.0 eq), followed by HATU (17.4 mg, 45.7 μmol, 2.0 eq) and DIEA (14.8 mg, 114 μmol, 19.9 μL, 5.0 eq). The resulting mixture was stirred at 20° C. for 16 h. LCMS indicated that the starting material was completely consumed, and the desired mass was detected. The mixture was treated with H₂O (1.0 mL) and extracted with EtOAc (1.0 mL×3). The combined organic layers were dried over Na₂SO₄, filtered, and concentrated under vacuum to give a residue which was purified by preparative HPLC (Phenomenex Luna C18 column (80× 30 mm, 3 μm); flow rate: 25 mL/min; gradient: 1%-30% B over 8 min; mobile phase A: 0.04% aqueous HCl, mobile phase B: acetonitrile). (S)—N-(1-(7-Chloroquinolin-5-yl)cyclopropyl)-2-methyl-5-((1-methylazetidin-2-yl)methoxy)benzamide (2.00 mg, 4.36 μmol, 19% yield, HCl salt) was obtained as a white solid. M+H⁺=436.0 (LCMS); ¹H NMR (400 MHZ, DMSO-d₆) δ 10.77-10.14 (m, 1H), 9.28 (s, 1H), 9.21 (br d, J=8.6 Hz, 1H), 9.04 (d, J=4.0 Hz, 1H), 8.09 (d, J=1.5 Hz, 1H), 7.90 (d, J=2.0 Hz, 1H), 7.75 (dd, J=4.4, 8.6 Hz, 1H), 7.09 (d, J=8.4 Hz, 1H), 6.98-6.87 (m, 1H), 6.73 (d, J=2.6 Hz, 1H), 4.68-4.55 (m, 1H), 4.33 (br dd, J=7.8, 11.3 Hz, 1H), 4.26-4.18 (m, 1H), 3.99 (br dd, J=4.6, 10.0 Hz, 1H), 3.88-3.83 (m, 1H), 2.81 (d, J=5.0 Hz, 3H), 2.38-2.27 (m, 2H), 1.95 (s, 3H), 1.38 (br s, 2H), 1.30 (br s, 2H).

### Example 390: (S)—N-(1-(7-Aminoquinolin-5-yl)cyclopropyl)-2-methyl-5-((1-methylazetidin-2-yl)methoxy)benzamide (Compound 729)

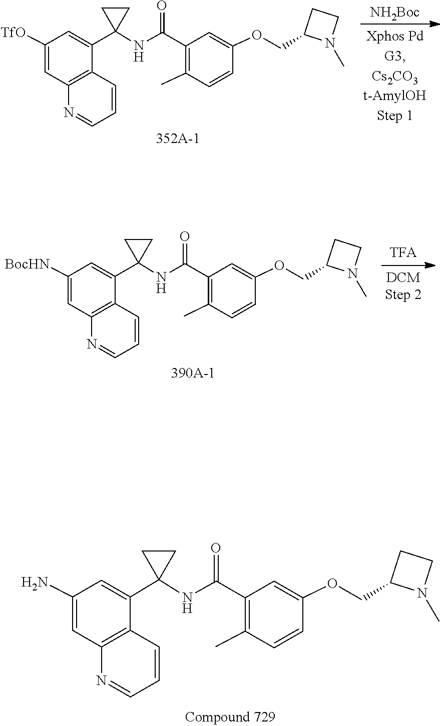 [SMILES: Cc1ccc(OC[C@@H]2CCN2C)cc1C(=O)NC1(c2cc(OS(=O)(=O)C(F)(F)F)cc3ncccc23)CC1 ; Cc1ccc(OC[C@@H]2CCN2C)cc1C(=O)NC1(c2cc(NC(=O)OC(C)(C)C)cc3ncccc23)CC1 ; Cc1ccc(OC[C@@H]2CCN2C)cc1C(=O)NC1(c2cc(N)cc3ncccc23)CC1 ; "NH2Boc"]

### Step 1: tert-Butyl(S)-(5-(1-(2-methyl-5-((1-methylazetidin-2-yl)methoxy)benzamido)cyclopropyl)quinolin-7-yl)carbamate (390A-1)

To a mixture of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (250 mg, 455 μmol, 1.0 eq) and tert-butyl carbamate (63.9 mg, 546 μmol, 1.2 eq) in t-AmylOH (13 mL) were added Cs₂CO₃ (296 mg, 910 μmol, 2.0 eq) and Xphos Pd G3 (38.5 mg, 45.5 μmol, 0.1 eq). The mixture was degassed, purged with N₂ three times, and stirred at 80° C. for 14 h under a N₂ atmosphere. LCMS indicated that the starting material was completely consumed, and the desired mass was detected. The reaction mixture was allowed to cool to room temperature, poured into H₂O (12 mL), and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/2. tert-Butyl(S)-(5-(1-(2-methyl-5-((1-methylazetidin-2-yl)methoxy)benzamido)cyclopropyl)quinolin-7-yl)carbamate (170 mg, 329 μmol, 72% yield) was obtained as a yellow oil. M+H⁺=517.3 (LCMS).

### Step 2: (S)—N-(1-(7-Aminoquinolin-5-yl)cyclopropyl)-2-methyl-5-((1-methylazetidin-2-yl) methoxy)benzamide (Compound 729)

To a solution of tert-butyl(S)-(5-(1-(2-methyl-5-((1-methylazetidin-2-yl)methoxy)benzamido)cyclopropyl)quinolin-7-yl)carbamate (170 mg, 329 μmol, 1.0 eq) in DCM (4.0 mL) was added TFA (1.0 mL). The mixture was stirred at 25° C. for 1 h. LCMS indicated that the starting material was completely consumed, and the desired mass was detected. The mixture was concentrated under vacuum at 30° C. to give a residue which was purified by preparative HPLC (Waters Xbridge BEH C18 column (100×30 mm, 10 μm); flow rate: 25 mL/min; gradient: 20%-50% B over 8 min; mobile phase A: 10 mM aqueous NH₄HCO₃, mobile phase B: acetonitrile) to give(S)—N-(1-(7-aminoquinolin-5-yl)cyclopropyl)-2-methyl-5-((1-methylazetidin-2-yl)methoxy)benzamide (5.10 mg, 8.10 μmol, 3% yield) as a yellow solid. M+H⁺=417.2 (LCMS); ¹H NMR (400 MHZ, DMSO-d₆) δ 9.00 (s, 1H), 8.74 (d, J=7.4 Hz, 1H), 8.58 (dd, J=1.6, 4.2 Hz, 1H), 7.32 (d, J=2.3 Hz, 1H), 7.11 (dd, J=4.3, 8.4 Hz, 1H), 7.03 (d, J=8.4 Hz, 1H), 6.86-6.81 (m, 2H), 6.61 (d, J=2.6 Hz, 1H), 5.75 (d, J=6.4 Hz, 2H), 3.87 (d, J=5.5 Hz, 2H), 3.27-3.21 (m, 2H), 2.77-2.67 (m, 1H), 2.22 (s, 3H), 1.99-1.97 (m, 3H), 1.96-1.90 (m, 1H), 1.89-1.82 (m, 1H), 1.32-1.27 (m, 2H), 1.13-1.08 (m, 2H).

### Example 391: (S)—N-(1-(7-(Dimethylamino)quinolin-5-yl)cyclopropyl)-2-methyl-5-((1-methylazetidin-2-yl)methoxy)benzamide (Compound 627)

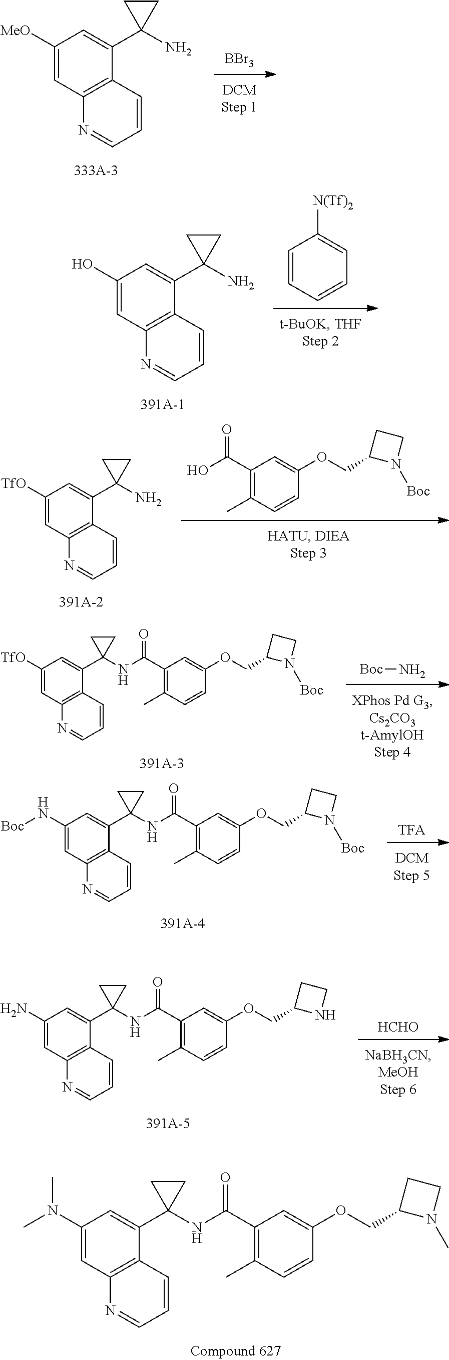

### Step 1: 5-(1-Aminocyclopropyl)quinolin-7-ol (391A-1)

To a solution of 1-(7-methoxyquinolin-5-yl)cyclopropan-1-amine (1.50 g, 5.98 mmol, 1.0 eq) in DCM (50 mL) was added a solution of BBr₃ (22.5 g, 90.0 mmol, 8.7 mL, 15 eq) in DCM (20 mL) dropwise at −78° C. under a N₂ atmosphere. The resulting mixture was stirred at the same temperature for 2 h, then warmed to 20° C. and stirred for another 12 h. LCMS indicated that the starting material was completely consumed, and the desired mass was detected. The reaction mixture was concentrated under vacuum to give a residue, which was diluted with MeOH (20 mL) at 0° C. and treated with NH₃·H₂O to adjust the pH 8. The mixture was concentrated under vacuum to give a residue which was purified by flash silica gel chromatography using a gradient of EtOAc/petroleum ether from 0/1 to 1/1, followed by DCM/MeOH from 100/1 to 10/1. 5-(1-Aminocyclopropyl)quinolin-7-ol (1.73 g, 8.64 mmol, 72% yield) was obtained as a yellow solid. M+H⁺=201.3 (LCMS); ¹H NMR (400 MHZ, DMSO-d₆) δ 10.51-10.23 (m, 1H), 8.90-8.78 (m, 1H), 8.62 (d, J=8.5 Hz, 1H), 7.43 (dd, J=4.3, 8.5 Hz, 1H), 7.38 (d, J=2.4 Hz, 1H), 7.32 (d, J=2.3 Hz, 1H), 4.09 (q, J=5.1 Hz, 2H), 1.55-1.48 (m, 2H), 1.28-1.15 (m, 2H).

### Step 2: 5-(1-Aminocyclopropyl)quinolin-7-yl trifluoromethanesulfonate (391A-2)

To a solution of 5-(1-aminocyclopropyl)quinolin-7-ol (1.40 g, 6.99 mmol, 1.0 eq) in THF (50 mL) was added 1-BuOK (1.57 g, 14.0 mmol, 2.0 eq) at 0° C. under a N₂ atmosphere. The mixture was stirred at 0° C. for 15 min. 1,1,1-Trifluoro-N-phenyl-N-(trifluoromethylsulfonyl)methane sulfonamide (5.00 g, 14.0 mmol, 2.0 eq) was added in portions. The resulting reaction mixture was stirred at 20° C. for 6 h. LCMS indicated that the starting material was completely consumed, and the desired mass was detected. The reaction mixture was poured into H₂O (50 mL) and extracted with EtOAc (50 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. 5-(1-Aminocyclopropyl)quinolin-7-yl trifluoromethanesulfonate (1.25 g, 3.76 mmol, 54% yield) was obtained as a yellow oil. M+H⁺=333.2 (LCMS); ¹H NMR (400 MHZ, DMSO-d₆) δ 9.05 (dd, J=1.6, 4.1 Hz, 1H), 8.96 (d, J=8.5 Hz, 1H), 8.05 (d, J=2.5 Hz, 1H), 7.74 (dd, J=4.3, 8.6 Hz, 1H), 7.67 (d, J=2.6 Hz, 1H), 4.20-3.98 (m, 2H), 3.17 (s, 3H), 1.22-1.15 (m, 2H), 1.04-0.95 (m, 2H).

### Step 3: tert-Butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (391A-3)

To a solution of 5-(1-aminocyclopropyl)quinolin-7-yl trifluoromethanesulfonate (517 mg, 1.56 mmol, 1.0 eq) and(S)-5-((1-(tert-butoxycarbonyl)azetidin-2-yl)methoxy)-2-methylbenzoic acid (500 mg, 1.56 mmol, 1.0 eq) in DMF (13 mL) were added DIEA (603 mg, 4.67 mmol, 813 μL, 3.0 eq) and HATU (1.48 g, 3.89 mmol, 2.5 eq). The mixture was stirred at 20° C. for 16 h. LCMS indicated that the starting material was completely consumed, and the desired mass was detected. The reaction mixture was poured into H₂O (30 mL) and extracted with EtOAC (30 mL×5). The combined organic layers were dried over Na₂SO₄, filtered, and concentrated under vacuum to give a residue which was purified by flash silica gel chromatography using a gradient of EtOAc/petroleum ether from 1/100 to 1/0. tert-Butyl(S)-2-((4-methyl-3-((1-(7-(((trifluoromethyl)sulfonyl)oxy)quinolin-5-yl)cyclopropyl)carbamoyl) phenoxy)methyl)azetidine-1-carboxylate (840 mg, 1.32 mmol, 85% yield) was obtained as a yellow gum. M+H⁺=636.4 (LCMS).

### Step 4: tert-Butyl(S)-2-((3-((1-(7-((tert-butoxycarbonyl)amino)quinolin-5-yl)cyclopropyl)carbamoyl)-4-methylphenoxy)methyl)azetidine-1-carboxylate (391A-4)

To a mixture of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (200 mg, 315 μmol, 1.0 eq) and tert-butyl carbamate (44.2 mg, 378 μmol, 1.2 eq) in 1-AmylOH (10 mL) were added Cs₂CO₃ (205 mg, 629 μmol, 2.0 eq) and XPhos Pd G3 (26.6 mg, 31.5 μmol, 0.1 eq). The mixture was degassed and purged with N₂ three times, and the mixture was stirred at 80° C. for 14 h under a N₂ atmosphere. LCMS indicated that the starting material was completely consumed, and the desired mass was detected. The reaction mixture was allowed to cool to room temperature, poured into H₂O (10 mL) and extracted with DCM (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/2. tert-Butyl(S)-2-((3-((1-(7-((tert-butoxycarbonyl)amino)quinolin-5-yl)cyclopropyl)carbamoyl)-4-methylphenoxy)methyl)azetidine-1-carboxylate (130 mg, 216 μmol, 69% yield) was obtained as a colorless oil. M+H⁺=603.4 (LCMS); ¹H NMR (400 MHZ, CDCl₃) δ 9.02 (d, J=8.4 Hz, 1H), 8.87 (dd, J=1.6, 4.2 Hz, 1H), 8.04 (br d, J=2.4 Hz, 2H), 7.39 (dd, J=4.3, 8.5 Hz, 1H), 7.02 (d, J=8.5 Hz, 1H), 6.87-6.80 (m, 2H), 6.75 (d, J=2.6 Hz, 1H), 6.65 (br s, 1H), 4.44 (br d, J=7.2 Hz, 1H), 4.23-4.16 (m, 1H), 4.02 (dd, J=2.6, 10.4 Hz, 1H), 3.86 (br t, J=7.5 Hz, 2H), 2.35-2.17 (m, 2H), 2.14 (s, 3H), 1.61 (br s, 9H), 1.41-1.38 (m, 2H), 1.34 (br s, 9H), 1.30-1.24 (m, 2H).

### Step 5: (S)—N-(1-(7-Aminoquinolin-5-yl)cyclopropyl)-5-(azetidin-2-ylmethoxy)-2-methylbenzamide (391A-5)

To a solution of tert-butyl(S)-2-((3-((1-(7-((tert-butoxycarbonyl)amino)quinolin-5-yl)cyclopropyl)carbamoyl)-4-methylphenoxy)methyl)azetidine-1-carboxylate (130 mg, 132 μmol, 1.0 eq) in DCM (4.0 mL) was added TFA (1.0 mL). The mixture was stirred at 20° C. for 1 h. LCMS indicated that the starting material was completely consumed, and the desired mass was detected. The mixture was concentrated under vacuum at 20° C. to give the crude product(S)—N-(1-(7-aminoquinolin-5-yl)cyclopropyl)-5-(azetidin-2-ylmethoxy)-2-methylbenzamide (170 mg, TFA salt) as a yellow oil. M+H⁺==403.2 (LCMS).

### Step 6: (S)—N-(1-(7-(Dimethylamino)quinolin-5-yl)cyclopropyl)-2-methyl-5-((1-methylazetidin-2-yl)methoxy)benzamide (Compound 627)

To a solution of(S)—N-(1-(7-aminoquinolin-5-yl)cyclopropyl)-5-(azetidin-2-ylmethoxy)-2-methylbenzamide (150 mg, 290 μmol, 1.0 eq) in MeOH (5.0 mL) was added TEA (40.4 μL) followed by formaldehyde (236 mg, 2.90 mmol, 216 μL, 37% purity in water, 10 eq). The resulting mixture was adjusted to pH 6 with a small amount of AcOH. The mixture was stirred at 20° C. for 30 min, then NaBH₃CN (91.3 mg, 1.45 mmol, 5.0 eq) was added. The reaction mixture was stirred at 25° C. for another 2 h. LCMS indicated that the starting material was completely consumed, and the desired mass was detected. The reaction mixture was poured into H₂O (5.0 mL) and extracted with DCM (3.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 1%-25% B over 8 min; mobile phase A: 0.04% aqueous HCl, mobile phase B: acetonitrile). (S)—N-(1-(7-(Dimethylamino)quinolin-5-yl)cyclopropyl)-2-methyl-5-((1-methylazetidin-2-yl)methoxy)benzamide (52.9 mg, 117 μmol, 40% yield, HCl salt) was obtained as a yellow solid. M+H⁺=445.2 (LCMS). ¹H NMR (400 MHZ, DMSO-d₆) δ 10.86-10.77 (m, 1H), 9.43 (br d, J=8.0 Hz, 1H), 9.26 (s, 1H), 8.86 (dd, J=1.1, 5.5 Hz, 1H), 7.73 (d, J=2.4 Hz, 1H), 7.59 (dd, J=5.6, 8.2 Hz, 1H), 7.10 (d, J=8.4 Hz, 1H), 7.00 (d, J=2.1 Hz, 1H), 6.93 (dd, J=2.7, 8.5 Hz, 1H), 6.82-6.73 (m, 1H), 4.71-4.57 (m, 1H), 4.43-4.34 (m, 1H), 4.22 (dd, J=3.3, 11.4 Hz, 1H), 4.05-3.94 (m, 1H), 3.91-3.79 (m, 1H), 3.21 (s, 6H), 2.81 (d, J=3.9 Hz, 3H), 2.36-2.26 (m, 2H), 1.98 (s, 3H), 1.39 (br s, 2H), 1.33 (br s, 2H).

### Example 392: (S)—N-(1-(2-(Dimethylamino)-7-fluoroquinolin-4-yl)cyclopropyl)-2-methyl-5-((1-methylazetidin-2-yl)methoxy)benzamide (Compound 611)

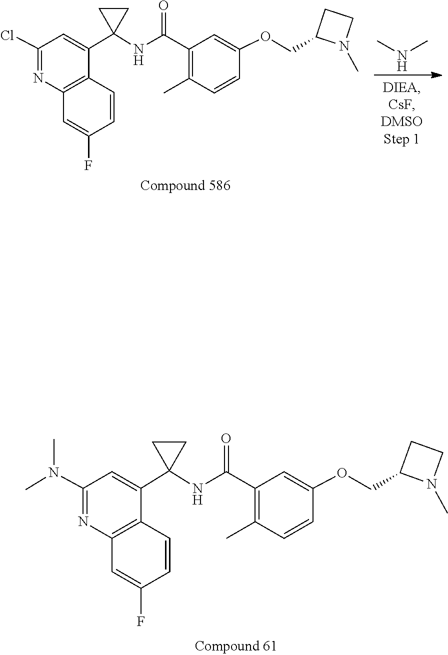

### Step 1: (S)—N-(1-(2-(Dimethylamino)-7-fluoroquinolin-4-yl)cyclopropyl)-2-methyl-5-((1-methylazetidin-2-yl)methoxy)benzamide (Compound 611)

To a solution of(S)—N-(1-(2-chloro-7-fluoroquinolin-4-yl)cyclopropyl)-2-methyl-5-((1-methylazetidin-2-yl)methoxy)benzamide (80.0 mg, 176 μmol, 1.0 eq), dimethylamine (2 M in THF, 176 μL, 2.0 eq) in DMSO (1.0 mL) were added DIEA (45.5 mg, 352 μmol, 61.4 μL, 2.0 eq) and CsF (53.5 mg, 352 μmol, 2.0 eq) at 20° C. The mixture was stirred at 120° C. for 12 h. LCMS indicated that the starting material was completely consumed, and the desired mass was detected. The reaction mixture was allowed to cool to room temperature and filtered. The filtrate was purified by preparative HPLC (Phenomenex Luna C18 column (80×30 mm, 3 μm); flow rate: 25 mL/min; gradient: 5%-35% B over 8 min; mobile phase A: 0.04% aqueous HCl, mobile phase B: acetonitrile). (S)—N-(1-(2-(Dimethylamino)-7-fluoroquinolin-4-yl)cyclopropyl)-2-methyl-5-((1-methylazetidin-2-yl)methoxy)benzamide (5.50 mg, 10.5 μmol, 6% yield, HCl salt) was obtained as a yellow solid. M+H⁺=463.2 (LCMS); ¹H NMR (400 MHZ, DMSO-d₆) δ 13.12-12.61 (m, 1H), 11.03-10.89 (m, 1H), 9.38 (s, 1H), 8.60 (dd, J=6.0, 9.1 Hz, 1H), 8.26-8.10 (m, 1H), 7.52-7.43 (m, 1H), 7.37 (s, 1H), 7.12 (d, J=8.5 Hz, 1H), 6.94 (dd, J=2.7, 8.4 Hz, 1H), 6.75 (d, J=2.6 Hz, 1H), 4.63 (br s, 1H), 4.41 (dd, J=8.1, 11.1 Hz, 1H), 4.22 (dd, J=3.1, 11.1 Hz, 1H), 4.05-3.96 (m, 2H), 3.46 (s, 6H), 2.54 (s, 3H), 2.42-2.25 (m, 2H), 1.99 (s, 3H), 1.40 (s, 4H).

### Example 393: (S)-2-Methyl-5-((1-methylazetidin-2-yl)methoxy)-N-(1-(7-(propylamino)quinolin-5-yl)cyclopropyl)benzamide (Compound 743)

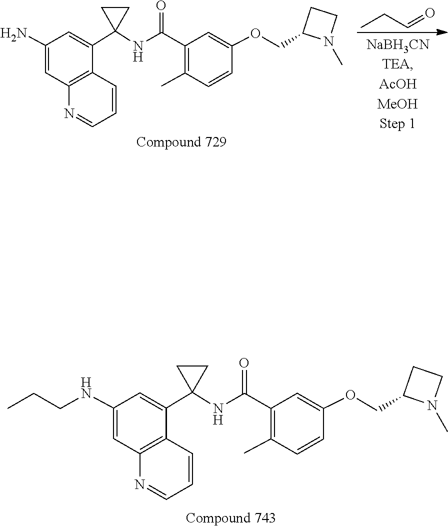

### Step 1: (S)-2-Methyl-5-((1-methylazetidin-2-yl)methoxy)-N-(1-(7-(propylamino)quinolin-5-yl)cyclopropyl)benzamide (Compound 743)

To a solution of(S)—N-(1-(7-aminoquinolin-5-yl)cyclopropyl)-2-methyl-5-((1-methylazetidin-2-yl)methoxy)benzamide (60.0 mg, 144 μmol, 1.0 eq) in MeOH (3.0 mL) was added TEA (20.0 μL), followed by propionaldehyde (8.37 mg, 144 μmol, 10.5 μL, 10 eq). The resulting mixture was adjusted to pH 6 with a small amount of AcOH. NaBH₃CN (45.3 mg, 720 μmol, 5.0 eq) was added. The resulting mixture was stirred at 25° C. for 1 h. LCMS indicated that the starting material was completely consumed, and the desired mass was detected. The reaction mixture was poured into H₂O (5.0 mL) and extracted with DCM (3.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 1%-25% B over 8 min; mobile phase A: 0.04% aqueous HCl, mobile phase B: acetonitrile). (S)-2-Methyl-5-((1-methylazetidin-2-yl)methoxy)-N-(1-(7-(propylamino)quinolin-5-yl)cyclopropyl)benzamide (1.9 mg, 3.84 μmol, 3% yield, HCl salt) was obtained as a yellow solid. M+H⁺=459.1 (LCMS); ¹H NMR (400 MHz, DMSO-d₆) δ 10.23-10.14 (m, 1H), 9.31-9.22 (m, 1H), 9.17 (s, 1H), 8.77 (br d, J=6.1 Hz, 1H), 7.64-7.59 (m, 1H), 7.54-7.47 (m, 1H), 7.11 (d, J=8.8 Hz, 1H), 6.93 (dd, J=2.7, 8.3 Hz, 1H), 6.76 (d, J=2.9 Hz, 1H), 6.72 (br s, 1H), 4.67-4.57 (m, 1H), 4.33-4.20 (m, 2H), 4.07-3.96 (m, 1H), 3.91-3.82 (m, 1H), 3.19-3.13 (m, 2H), 2.87-2.79 (m, 3H), 2.37-2.28 (m, 2H), 1.97 (s, 3H), 1.71-1.63 (m, 2H), 1.40-1.33 (m, 2H), 1.23-1.17 (m, 2H), 1.00 (t, J=7.4 Hz, 3H).

### Example 394: (S)—N-(1-(7-Acetamidoquinolin-5-yl)cyclopropyl)-2-methyl-5-((1-methylazetidin-2-yl)methoxy)benzamide (Compound 744)

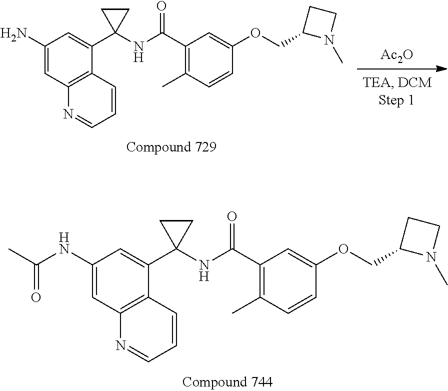

### Step 1: (S)—N-(1-(7-Acetamidoquinolin-5-yl)cyclopropyl)-2-methyl-5-((1-methylazetidin-2-yl)methoxy)benzamide (Compound 744)

To a solution of(S)—N-(1-(7-aminoquinolin-5-yl)cyclopropyl)-2-methyl-5-((1-methylazetidin-2-yl)methoxy)benzamide (50 mg, 120 μmol, 1.0 eq) in DCM (5 mL) were added TEA (48.6 mg, 480 μmol, 66.8 μL, 4.0 eq) and Ac₂O (24.5 mg, 240 μmol, 22.5 μL, 2.0 eq) at 0° C. The mixture was stirred at 25° C. for 3 h. LCMS indicated that the starting material was completely consumed, and the desired mass was detected. The reaction mixture was poured into H₂O (5.0 mL) and extracted with DCM (3.0 mL×3). The combined organic layers were dried over Na₂SO₄, filtered, and concentrated under vacuum to give a residue which was purified by preparative HPLC (Phenomenex C18 column (75×30 mm, 3 μm); flow rate: 25 mL/min; gradient: 10%-40% B over 8 min; mobile phase A: 0.1% aqueous TFA, mobile phase B: acetonitrile). (S)—N-(1-(7-Acetamidoquinolin-5-yl)cyclopropyl)-2-methyl-5-((1-methyl azetidin-2-yl)methoxy)benzamide (4.50 mg, 7.86 μmol, 7% yield) was obtained as a white solid. M+H⁺=459.1 (LCMS); ¹H NMR (400 MHZ, DMSO-d₆) δ 9.00 (s, 1H), 8.74 (d, J=7.4 Hz, 1H), 8.58 (dd, J=1.6, 4.2 Hz, 1H), 7.32 (d, J=2.3 Hz, 1H), 7.11 (dd, J=4.3, 8.4 Hz, 1H), 7.03 (d, J=8.4 Hz, 1H), 6.86-6.81 (m, 2H), 6.61 (d, J=2.6 Hz, 1H), 5.75 (d, J=6.4 Hz, 2H), 3.87 (d, J=5.5 Hz, 2H), 3.27-3.21 (m, 2H), 2.77-2.67 (m, 1H), 2.22 (s, 3H), 1.99-1.97 (m, 3H), 1.96-1.90 (m, 1H), 1.89-1.82 (m, 1H), 1.32-1.27 (m, 2H), 1.13-1.08 (m, 2H)

### Example 395: (S)-2-Methyl-5-((1-methylazetidin-2-yl)methoxy)-N-(1-(7-(methylsulfonamido)quinolin-5-yl)cyclopropyl)benzamide (Compound 786)

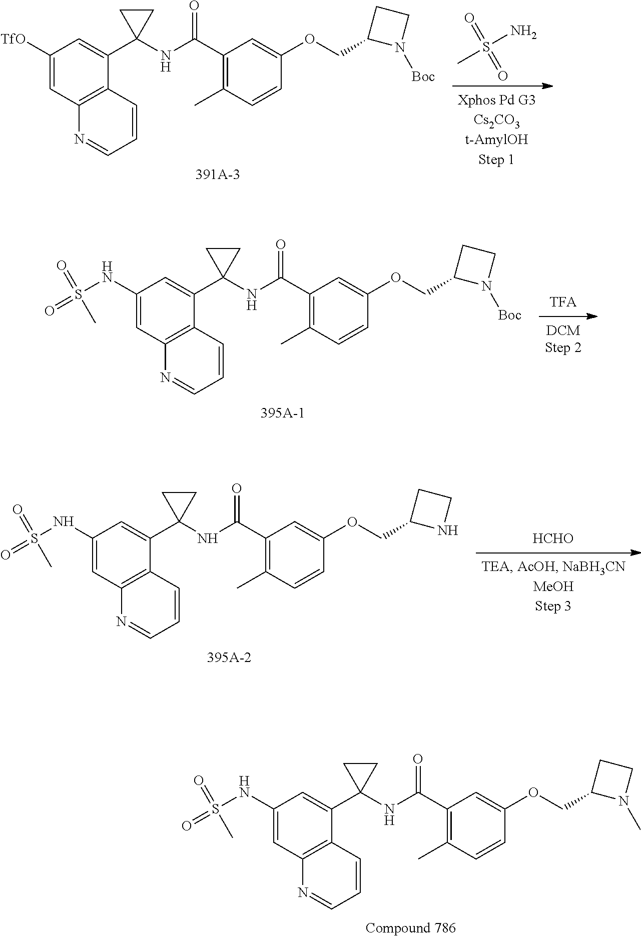

### Step 1: tert-Butyl(S)-2-((4-methyl-3-((1-(7-(methylsulfonamido)quinolin-5-yl)cyclopropyl)carbamoyl)phenoxy)methyl)azetidine-1-carboxylate (395A-1)

To a mixture of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (250 mg, 393 μmol, 1.0 eq) and methanesulfonamide (46.3 mg, 472 μmol, 1.2 eq) in t-AmylOH (10 mL) were added Cs₂CO₃ (256 mg, 787 μmol, 2.0 eq) and XPhos Pd G3 (33.3 mg, 39.3 μmol, 0.1 eq). The mixture was degassed and purged with N₂ three times, then the mixture was stirred at 80° C. for 14 h under a N₂ atmosphere. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/2. tert-Butyl 1 (S)-2-((4-methyl-3-((1-(7-(methylsulfonamido)quinolin-5-yl)cyclopropyl)carbamoyl)phenoxy)methyl)azetidine-1-carboxylate (210 mg, crude) was obtained as a yellow oil. M+H⁺=581.4 (LCMS); ¹H NMR (400 MHz, DMSO-d₆) δ 10.29-10.05 (m, 1H), 9.11 (d, J=19.6 Hz, 1H), 9.00-8.87 (m, 1H), 8.86-8.71 (m, 1H), 7.84-7.68 (m, 1H), 7.51-7.44 (m, 1H), 7.35-7.14 (m, 1H), 7.05 (d, J=8.5 Hz, 1H), 6.88 (dd, J=2.4, 8.4 Hz, 1H), 6.66 (d, J=1.8 Hz, 1H), 4.38 (br dd, J=2.9, 7.6 Hz, 1H), 4.14 (dd, J=4.7, 10.3 Hz, 1H), 4.01-3.95 (m, 1H), 3.73 (br s, 2H), 3.30-3.28 (m, 6H), 2.30-2.22 (m, 1H), 2.20-2.15 (m, 9H), 2.11-2.03 (m, 1H), 1.23 (s, 2H), 1.16 (br d, J=7.0 Hz, 2H).

### Step 2: (S)-5-(Azetidin-2-ylmethoxy)-2-methyl-N-(1-(7-(methylsulfonamido)quinolin-5-yl)cyclopropyl)benzamide (395A-2)

To a solution of tert-butyl(S)-2-((4-methyl-3-((1-(7-(methylsulfonamido)quinolin-5-yl)cyclo propyl)carbamoyl)phenoxy)methyl)azetidine-1-carboxylate (200 mg, 344 μmol, 1.0 eq) in DCM (5.0 mL) was added TFA (1.0 mL). The mixture was stirred at 20° C. for 1 h. LCMS indicated that the starting material was completely consumed, and the desired mass was detected. The mixture was concentrated under vacuum at 20° C. to give the crude product(S)-5-(azetidin-2-ylmethoxy)-2-methyl-N-(1-(7-(methylsulfonamido)quinolin-5-yl)cyclopropyl)benzamide (200 mg, TFA salt) as a yellow oil. M+H⁺=481.3 (LCMS).

### Step 3: (S)-2-Methyl-5-((1-methylazetidin-2-yl)methoxy)-N-(1-(7-(methylsulfonamido)quinolin-5-yl)cyclopropyl)benzamide (Compound 786)

To a solution of(S)-5-(azetidin-2-ylmethoxy)-2-methyl-N-(1-(7-(methylsulfonamido)quinolin-5-yl)cyclopropyl)benzamide (200 mg, 336 μmol, 1.0 eq) in MeOH (6.0 mL) was added TEA (47 μL), followed by formaldehyde (20.2 mg, 673 μmol, 19.0 μL, 37% purity in water, 2.0 eq). The resulting mixture was adjusted to pH 6 with a small amount of AcOH. The mixture was stirred at 20° C. for 30 min, then NaBH₃CN (63.4 mg, 1.01 mmol, 3.0 eq) was added. The reaction mixture was stirred at 25° C. for 2 h. LCMS indicated that the starting material was completely consumed, and the desired mass was detected. The reaction mixture was poured into H₂O (5.0 mL) and extracted with DCM (3.0 mL×3). The combined organic layers were dried over Na₂SO₄, filtered, and concentrated under vacuum to give a residue which was purified by preparative HPLC (Phenomenex luna C18 column (100× 40 mm, 5 μm); flow rate: 25 mL/min; gradient: 1%-25% B over 8 min; mobile phase A: 0.1% aqueous TFA, mobile phase B: acetonitrile). The compound was further purified by SFC separation (DAICEL CHIRALCEL OJ (250 mm×30 mm, 10 μm); flow rate: 30 mL/min; gradient: 20% B over 17 min; mobile phase A: heptane, mobile phase B: i-PrOH (0.1% NH₃·H₂O)). (S)-2-Methyl-5-((1-methylazetidin-2-yl)methoxy)-N-(1-(7-(methylsulfonamido)quinolin-5-yl)cyclopropyl)benzamide (10.8 mg, 21.8 μmol, 7% yield) was obtained as a yellow solid. M+H⁺=495.4 (LCMS); ¹H NMR (400 MHZ, DMSO-d₆) δ 9.11 (s, 1H), 8.96 (br d, J=8.1 Hz, 1H), 8.87-8.79 (m, 1H), 7.79 (d, J=2.0 Hz, 1H), 7.67 (s, 1H), 7.45 (dd, J=4.2, 8.4 Hz, 1H), 7.03 (d, J=8.5 Hz, 1H), 6.83 (dd, J=2.5, 8.4 Hz, 1H), 6.61 (d, J=2.5 Hz, 1H), 3.86 (d, J=5.4 Hz, 2H), 3.25-3.21 (m, 2H), 3.10 (s, 3H), 2.76-2.67 (m, 1H), 2.21 (s, 3H), 1.98-1.81 (m, 5H), 1.36 (br s, 2H), 1.16 (br s, 2H).

### Example 396: 2-Methyl-5-((S)-1-((S)-1-methylazetidin-2-yl) ethoxy)-N-(1-(7-vinylquinolin-5-yl)cyclopropyl)benzamide (Compound 757)

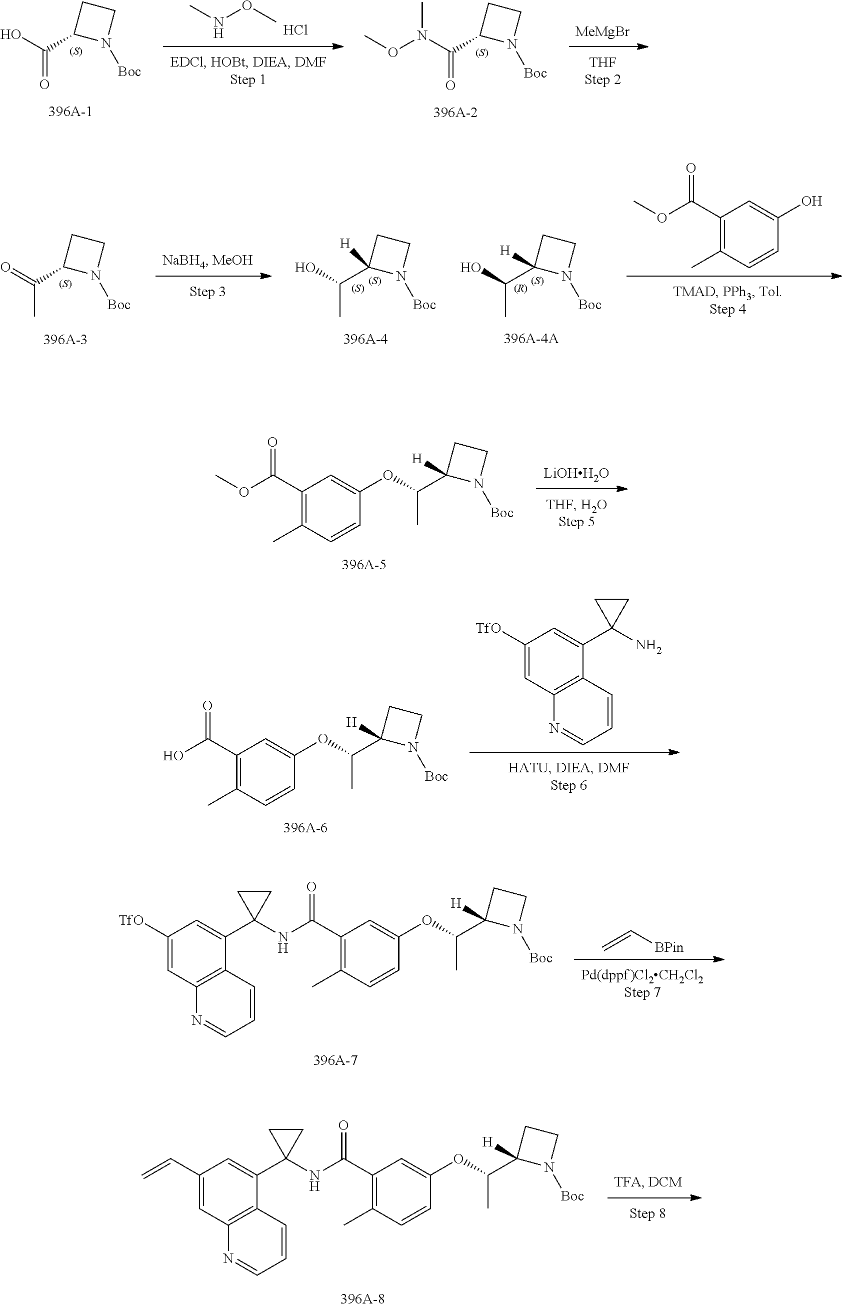

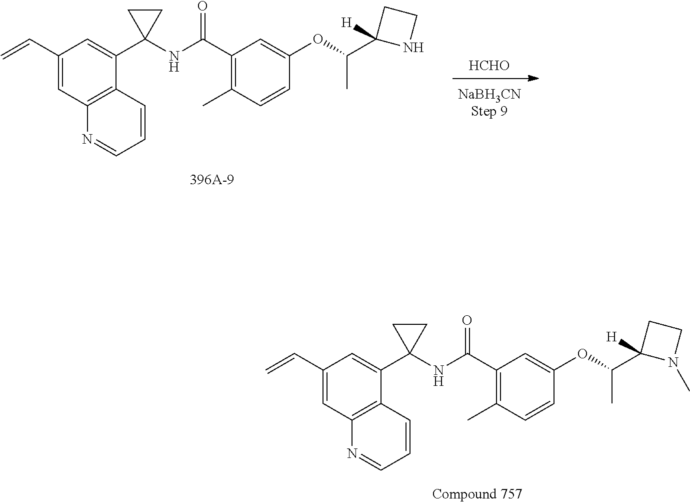 [SMILES: C=Cc1cc(C2(NC(=O)c3cc(O[C@@H](C)[C@@H]4CCN4)ccc3C)CC2)c2cccnc2c1 ; "HCHO" ; C=Cc1cc(C2(NC(=O)c3cc(O[C@@H](C)[C@@H]4CCN4C)ccc3C)CC2)c2cccnc2c1]

### Step 1: tert-Butyl(S)-2-(methoxy(methyl)carbamoyl)azetidine-1-carboxylate (396A-2)

To a solution of(S)-1-(tert-butoxycarbonyl)azetidine-2-carboxylic acid (4.00 g, 11.8 mmol, 1.0 eq) and N,O-dimethylhydroxylamine hydrochloride (2.33 g, 23.8 mmol, 1.2 eq) in DMF (40 mL) were added EDCI (4.57 g, 11.8 mmol, 1.0 eq), HOBt (3.22 g, 23.8 mmol, 1.2 eq) and NMM (2.41 g, 23.8 mmol, 2.62 mL, 1.2 eq) at 20° C. The mixture was stirred at 20° C. for 12 h. LCMS indicated that the starting material was completely consumed, and the desired mass was detected. The mixture was diluted with EtOAc (160 mL), then the solution was washed with HCl (1 M, 100 mL×1), NaOH (2 M, 100 mL×2) and brine (100 mL×3). The organic layers was dried over Na₂SO₄, filtered, and concentrated under vacuum to give tert-butyl(S)-2-(methoxy(methyl)carbamoyl)azetidine-1-carboxylate (2.95 g, 12.1 mmol, 60% yield) as a yellow solid. M−100+H⁺=145.2 (LCMS); ¹H NMR (400 MHZ, CDCl₃) δ 5.03 (br dd, J=5.4, 8.5 Hz, 1H), 4.04 (dt, J=6.4, 8.4 Hz, 1H), 3.86 (dt, J=5.6, 8.4 Hz, 1H), 3.70 (s, 3H), 3.21 (s, 3H), 2.46 (dtd, J=6.3, 9.0, 11.2 Hz, 1H), 2.18-2.04 (m, 1H), 1.42 (s, 9H).

### Step 2: tert-Butyl(S)-2-acetylazetidine-1-carboxylate (396A-3)

To a solution of tert-butyl(S)-2-(methoxy(methyl)carbamoyl)azetidine-1-carboxylate (2.95 g, 12.1 mmol, 1.0 eq) in THF (30 mL) were added MeMgBr (3 M in THF, 6.04 mL, 1.5 eq) at −78° C. The mixture was stirred at 20° C. for 12 h under a N₂ atmosphere. LCMS indicated that the starting material was completely consumed, and the desired mass was detected. The mixture was quenched with NH₄Cl (30 mL), the mixture was extracted with EtOAc (30 mL×3). The combined organic layers were dried over Na₂SO₄, filtered, and concentrated under vacuum to give a residue which was purified by flash silica gel chromatography using a gradient of EtOAc/petroleum ether from 0/1 to 3/7. tert-Butyl(S)-2-acetylazetidine-1-carboxylate (2.50 g) was obtained as a pale-yellow oil. M+Na⁺=222.1 (LCMS); ¹H NMR (400 MHZ, CDCl₃) δ 4.60 (dd, J=6.1, 9.5 Hz, 1H), 3.96-3.84 (m, 2H), 2.52-2.40 (m, 1H), 2.27 (s, 3H), 2.13 (tdd, J=6.4, 8.7, 11.6 Hz, 1H), 1.44 (s, 9H).

### Step 3: tert-Butyl(S)-2-((S)-1-hydroxyethyl)azetidine-1-carboxylate (396A-4) and tert-butyl(S)-2-((R)-1-hydroxyethyl)azetidine-1-carboxylate (396A-4A)

To a solution of tert-butyl(S)-2-acetylazetidine-1-carboxylate (2.51 g, 12.6 mmol, 1.0 eq) in MeOH (30 mL) was added NaBH₄ (571 mg, 15.1 mmol, 1.2 eq) at 0° C. The mixture was stirred at 0° C. for 30 min. LCMS indicated that the starting material was completely consumed, and the desired mass was detected. The mixture was quenched with NH₄Cl (30 mL), then extracted with EtOAc (30 mL×3). The combined organic layers were dried over Na₂SO₄. filtered, and concentrated under vacuum to give a residue which was purified by flash silica gel chromatography using a gradient of EtOAc/petroleum ether from 0/1 to 1/4. tert-Butyl(S)-2-((S)-1-hydroxyethyl)azetidine-1-carboxylate (897 mg, 4.46 mmol, 35% yield) was obtained as a colourless oil. M+Na⁺=224.1 (LCMS); ¹H NMR (400 MHZ, CDCl₃) δ 4.09-4.03 (m, 1H), 3.93-3.80 (m, 2H), 3.74 (dt, J=4.6, 8.9 Hz, 1H), 2.16 (dt, J=4.3, 7.8 Hz, 1H), 1.92-1.81 (m, 1H), 1.45 (s, 9H), 1.06 (d, J=6.3 Hz, 3H). tert-Butyl(S)-2-((R)-1-hydroxyethyl)azetidine-1-carboxylate (921 mg, 4.58 mmol, 36% yield) was obtained as a colourless oil. M+Na⁺=224.1 (LCMS); ¹H NMR (400 MHZ, CDCl₃) δ 4.33 (br s, 1H), 3.96 (br dd, J=2.1, 6.4 Hz, 1H), 3.85 (q, J=8.1 Hz, 1H), 3.77-3.67 (m, 1H), 2.09 (q, J=7.7 Hz, 2H), 1.45 (s, 9H), 1.15 (d, J=6.5 Hz, 3H).

### Step 4: tert-Butyl(S)-2-((S)-1-(3-(methoxycarbonyl)-4-methylphenoxy)ethyl)azetidine-1-carboxylate (396A-5)

To a solution of tert-butyl(S)-2-((S)-1-hydroxyethyl)azetidine-1-carboxylate (897 mg, 4.46 mmol, 1.0 eq) and methyl 5-hydroxy-2-methylbenzoate (740 mg, 4.45 mmol, 1.0 eq) in toluene (10 mL) were added TMAD (2.30 g, 13.3 mmol, 3.0 eq) and PPh₃ (3.50 g, 13.3 mmol, 3.0 eq) at 20° C. The mixture was stirred at 100° C. for 12 h. LCMS indicated that the starting material was completely consumed, and the desired mass was detected. The reaction was allowed to cool to room temperature. The mixture was concentrated under vacuum to give a residue which was purified by flash silica gel chromatography using a gradient of EtOAc/petroleum ether from 0/1 to 7/50. tert-Butyl(S)-2-((S)-1-(3-(methoxycarbonyl)-4-methylphenoxy)ethyl)azetidine-1-carboxylate (1.04 g, 2.98 mmol, 66% yield) was obtained as a colourless oil. M+H⁺=350.1 (LCMS); ¹H NMR (400 MHZ, CDCl₃) δ 7.51 (d, J=2.6 Hz, 1H), 7.16-7.08 (m, 1H), 7.03 (dd, J=2.3, 8.3 Hz, 1H), 4.84 (br d, J=1.5 Hz, 1H), 4.30-4.21 (m, 1H), 3.88 (s, 3H), 3.82 (br t, J=7.5 Hz, 2H), 2.51 (s, 3H), 2.45-2.32 (m, 1H), 2.29-2.15 (m, 1H), 1.36 (s, 9H), 1.20 (d, J=6.4 Hz, 3H).

### Step 5: 5-((S)-1-((S)-1-(tert-Butoxycarbonyl)azetidin-2-yl) ethoxy)-2-methylbenzoic acid (396A-6)

To a solution of tert-butyl(S)-2-((S)-1-(3-(methoxycarbonyl)-4-methylphenoxy)ethyl)azetidine-1-carboxylate (1.04 g, 2.98 mmol, 1.0 eq) in MeOH (3.0 mL) and THF (12 mL) was added NaOH (2 M, 5.95 mL, 4.0 eq) at 20° C. The mixture was stirred at 70° C. for 12 h. LCMS indicated that the starting material was completely consumed, and the desired mass was detected. The reaction was allowed to cool to room temperature. The mixture was concentrated under vacuum. The residue was diluted with H₂O (5.0 mL) and the mixture was extracted with EtOAc (5.0 mL×1). The organic layer was discarded. The aqueous layer was acidified to pH 5 by using HCl (1 M aqueous), then extracted with EtOAc (5.0 mL×3). The combined organic layers were dried over Na₂SO₄, filtered, and concentrated under vacuum to give 5-((S)-1-((S)-1-(tert-butoxycarbonyl)azetidin-2-yl) ethoxy)-2-methylbenzoic acid (994 mg, 2.87 mmol, 96% yield) as a yellow oil. M−100+H⁺==280.1 (LCMS).

### Step 6: tert-Butyl(S)-2-((S)-1-(4-methyl-3-((1-(7-(((trifluoromethyl)sulfonyl)oxy)quinolin-5-yl)cyclopropyl)carbamoyl)phenoxy)ethyl)azetidine-1-carboxylate (396A-7)

To a solution of 5-((S)-1-((S)-1-(tert-butoxycarbonyl)azetidin-2-yl) ethoxy)-2-methylbenzoic acid (75.0 mg, 223 μmol, 1.0 eq) and 5-(1-aminocyclopropyl)quinolin-7-yl trifluoromethanesulfonate (74.3 mg, 223 μmol, 1.0 eq) in DMF (1.0 mL) were added DIEA (86.7 mg, 670 μmol, 116 μL, 3.0 eq) and HBTU (169 mg, 447 μmol, 2.0 eq) at 20° C. The mixture was stirred at 20° C. for 2 h. LCMS indicated that the starting material was completely consumed, and the desired mass was detected. The residue was diluted with H₂O (5.0 mL), the aqueous layer was extracted with EtOAc (5.0 mL×3). The combined organic layers were dried over Na₂SO₄, filtered, and concentrated under vacuum to give a residue which was purified by preparative TLC (EtOAc/petroleum ether=1/1, R_{f}=0.65). tert-Butyl(S)-2-((S)-1-(4-methyl-3-((1-(7-(((trifluoromethyl)sulfonyl)oxy)quinolin-5-yl)cyclopropyl) carbamoyl)phenoxy)ethyl)azetidine-1-carboxylate (104 mg, 160 μmol, 71% yield) as a yellow oil.

### Step 7: tert-Butyl(S)-2-((S)-1-(4-methyl-3-((1-(7-vinylquinolin-5-yl)cyclopropyl) carbamoyl)phenoxy)ethyl)azetidine-1-carboxylate (396A-8)

To a solution of tert-butyl(S)-2-((S)-1-(4-methyl-3-((1-(7-(((trifluoromethyl)sulfonyl)oxy)quinolin-5-yl)cyclopropyl)carbamoyl)phenoxy)ethyl) azetidine-1-carboxylate (104 mg, 160 μmol, 1.0 eq) and 4,4,5,5-tetramethyl-2-vinyl-1,3,2-dioxaborolane (49.3 mg, 320 μmol, 2.0 eq) in a mixture of dioxane (2.0 mL) and H₂O (0.2 mL) were added Pd(dppf)Cl₂·CH₂Cl₂ (13.1 mg, 16.0 μmol, 0.1 eq) and Na₂CO₃ (39.0 mg, 368 μmol, 2.3 eq) at 20° C. The mixture was stirred at 80° C. for 2 h under a N₂ atmosphere. LCMS indicated that the starting material was completely consumed, and the desired mass was detected. The reaction mixture was allowed to cool to room temperature and was diluted with H₂O (5.0 mL). The aqueous layer was extracted with EtOAc (3.0 mL×3). The combined organic layers were dried over Na₂SO₄, filtered, and concentrated under vacuum to give a residue which was purified by preparative TLC (EtOAc/petroleum ether=1/1, R_{f}=0.59). tert-Butyl(S)-2-((S)-1-(4-methyl-3-((1-(7-vinylquinolin-5-yl)cyclopropyl)carbamoyl)phenoxy)ethyl)azetidine-1-carboxylate (56.0 mg, 106 μmol, 66% yield) was obtained as a pale yellow liquid.

### Step 8: 5-((S)-1-((S)-Azetidin-2-yl) ethoxy)-2-methyl-N-(1-(7-vinylquinolin-5-yl)cyclopropyl)benzamide (396A-9)

To a solution of tert-butyl(S)-2-((S)-1-(4-methyl-3-((1-(7-vinylquinolin-5-yl)cyclopropyl)carbamoyl)phenoxy)ethyl)azetidine-1-carboxylate (56.0 mg, 106 μmol, 1.0 eq) in DCM (1.5 mL) was added TFA (770 mg, 6.75 mmol, 0.5 mL, 63 eq) at 20° C. The mixture was stirred at 20° C. for 1 h. LCMS indicated that the starting material was completely consumed, and the desired mass was detected. The reaction mixture was concentrated under vacuum to give 5-((S)-1-((S)-azetidin-2-yl) ethoxy)-2-methyl-N-(1-(7-vinylquinolin-5-yl)cyclopropyl)benzamide (60.0 mg, crude, TFA salt) as a brown liquid, which was used in the next step without any further purification. M+H⁺=428.2 (LCMS).

### Step 9: 2-Methyl-5-((S)-1-((S)-1-methylazetidin-2-yl) ethoxy)-N-(1-(7-vinylquinolin-5-yl)cyclopropyl)benzamide (Compound 757)

To a solution of 5-((S)-1-((S)-azetidin-2-yl) ethoxy)-2-methyl-N-(1-(7-vinylquinolin-5-yl)cyclopropyl)benzamide (60.0 mg, 111 μmol, 1.0 eq, TFA salt) in MeOH (1.5 mL) was added TEA (15.0 μL), followed by formaldehyde (18.0 mg, 221 μmol, 6.34 μL, 37% purity in water, 2.0 eq). The resulting mixture was adjusted to pH 6 with a small amount of AcOH. The mixture was stirred at 20° C. for 30 min, then NaBH₃CN (20.9 mg, 332 μmol, 3.0 eq) was added. The resulting reaction mixture was stirred at 20° C. for another 12 h. LCMS indicated that the starting material was completely consumed, and the desired mass was detected. The reaction mixture was filtered, and the filtrate was purified by preparative HPLC (Phenomenex Gemini C18 column (80×30 mm, 3 μm); flow rate: 25 mL/min; gradient: 1%-25% B over 8 min; mobile phase A: 0.04% aqueous HCl, mobile phase B: acetonitrile). LCMS indicated that the purity was 68%, then the residue was further purified by preparative HPLC (Phenomenex Gemini C18 column (100×30 mm, 5 μm); flow rate: 25 mL/min; gradient: 1%-25% B over 8 min; mobile phase A: 0.04% aqueous HCl, mobile phase B: acetonitrile). 2-Methyl-5-((S)-1-((S)-1-methylazetidin-2-yl) ethoxy)-N-(1-(7-vinylquinolin-5-yl)cyclopropyl)benzamide (8.30 mg, 17.0 μmol, 15% yield, HCl salt) was obtained as a colorless gum. M+H⁺=442.4 (LCMS); ¹H NMR (400 MHZ, DMSO-d₆) δ 9.97-9.83 (m, 1H), 9.49 (br d, J=8.5 Hz, 1H), 9.30 (s, 1H), 9.12 (d, J=3.9 Hz, 1H), 8.21 (s, 1H), 8.11 (s, 1H), 7.89 (dd, J=4.8, 8.3 Hz, 1H), 7.13-7.00 (m, 2H), 6.93 (dd, J=2.6, 8.4 Hz, 1H), 6.77 (d, J=2.5 Hz, 1H), 6.19 (d, J=17.6 Hz, 1H), 5.62 (d, J=11.0 Hz, 1H), 4.85-4.77 (m, 1H), 4.50-4.39 (m, 1H), 4.04-3.97 (m, 1H), 3.81 (br dd, J=5.9, 9.4 Hz, 1H), 2.81 (d, J=4.9 Hz, 3H), 2.46-2.34 (m, 2H), 2.01-1.95 (m, 3H), 1.42 (br s, 2H), 1.33 (br d, J=5.0 Hz, 2H), 1.15-1.08 (m, 3H).

### Example 397: (S)-2-Methyl-5-(pyrrolidin-2-ylmethoxy)-N-(1-(7-vinylquinolin-5-yl)cyclopropyl)benzamide (Compound 713)

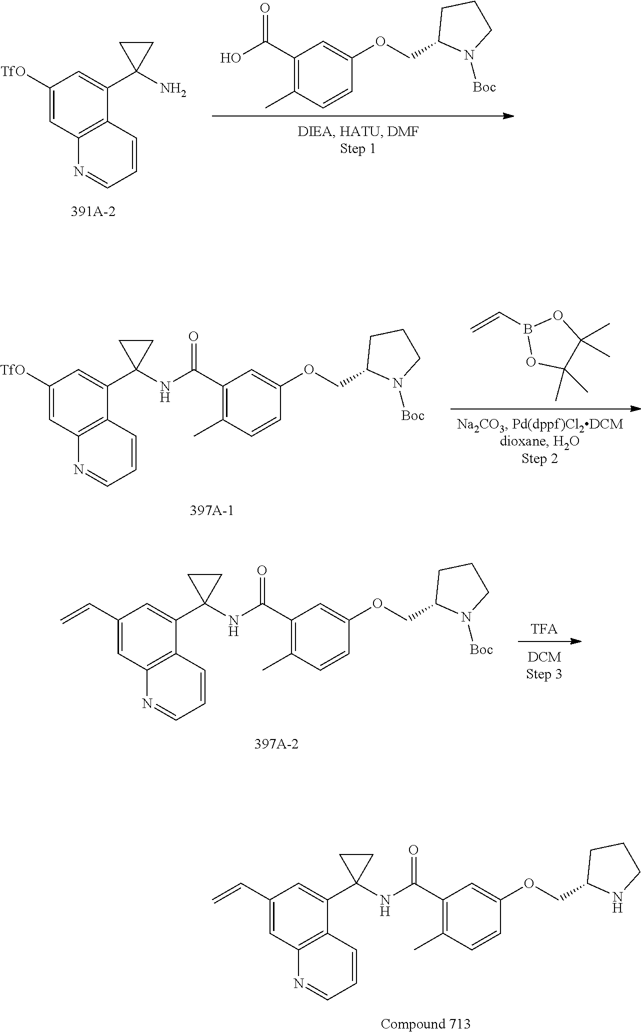

### Step 1: tert-Butyl(S)-2-((4-methyl-3-((1-(7-(((trifluoromethyl)sulfonyl)oxy)quinolin-5-yl)cyclopropyl)carbamoyl)phenoxy)methyl)pyrrolidine-1-carboxylate (397A-1)

To a solution of 5-(1-aminocyclopropyl)quinolin-7-yl trifluoromethanesulfonate (350 mg, 1.05 mmol, 1.0 eq) and(S)-5-((1-(tert-butoxycarbonyl)pyrrolidin-2-yl)methoxy)-2-methylbenzoic acid (353 mg, 1.05 mmol, 1.0 eq) in DMF (10 mL) were added DIEA (408 mg, 3.16 mmol, 3.0 eq) and HATU (1.00 g, 2.63 mmol, 2.5 eq). The mixture was stirred at 20° C. for 15 h. LCMS indicated that the starting material was completely consumed, and the desired mass was detected. The mixture was poured into H₂O (20 mL) and extracted with EtOAc (10 mL×4).

The combined organic layers were dried over Na₂SO₄, filtered, and concentrated under vacuum to give a residue which was purified by flash silica gel chromatography using a gradient of EtOAc/petroleum ether from 0/1 to 1/1. tert-Butyl(S)-2-((4-methyl-3-((1-(7-(((trifluoro methyl)sulfonyl)oxy)quinolin-5-yl)cyclopropy)carbamoyl)phenoxy)methyl)pyrolidine-1-carboxylate (520 mg, 800 μmol, 75% yield) was obtained as a white solid. M+H⁺=650.3 (LCMS).

### Step 2: tert-Butyl(S)-2-((4-methyl-3-((1-(7-vinylquinolin-5-yl)cyclopropyl)carbamoyl)phenoxy)methyl)pyrrolidine-1-carboxylate (397A-2)

To a stirred solution of tert-butyl(S)-2-((4-methyl-3-((1-(7-(((trifluoromethyl)sulfonyl)oxy)quinolin-5-yl)cyclopropyl)carbamoyl)phenoxy)methyl)pyrrolidine-1-carboxylate (250 mg, 385 μmol, 1.0 eq) and 4,4,5,5-tetramethyl-2-vinyl-1,3,2-dioxaborolane (65.2 mg, 423 μmol, 1.1 eq) in a mixture of dioxane (15 mL) and H₂O (3.0 mL) were added Pd (dppf) C12 (31.4 mg, 38.5 μmol, 0.1 eq), Na₂CO₃ (93.8 mg, δ85 μmol, 2.3 eq). The mixture was degassed, purged with N₂ three times, and stirred at 80° C. for 4 h under a N₂ atmosphere. LCMS indicated that the starting material was completely consumed, and the desired mass was detected. The reaction mixture was allowed to cool to room temperature, poured into H₂O (20 mL) and extracted with EtOAc (20 mL×4). The combined organic layers were dried over Na₂SO₄, filtered, and concentrated under vacuum to give a residue which was purified by flash silica gel chromatography using a gradient of EtOAc/petroleum ether from 0/1 to 7/3. tert-Butyl(S)-2-((4-methyl-3-((1-(7-vinylquinolin-5-yl)cyclopropyl)carbamoyl)phenoxy)methyl) pyrrolidine-1-carboxylate (156 mg, 296 μmol, 76% yield) was obtained as a white solid. M−56+H⁺=472.3 (LCMS).

### Step 3: (S)-2-Methyl-5-(pyrrolidin-2-ylmethoxy)-N-(1-(7-vinylquinolin-5-yl)cyclopropyl)benzamide (Compound 713)

To a solution of tert-butyl(S)-2-((4-methyl-3-((1-(7-vinylquinolin-5-yl)cyclopropyl)carbamoyl)phenoxy)methyl)pyrrolidine-1-carboxylate (46.0 mg, 87.1 μmol, 1.0 eq) in DCM (2.0 mL) was added TFA (770 mg, 6.75 mmol, 500 μL). The mixture was stirred at 25° C. for 1 h. LCMS indicated that the starting material was completely consumed, and the desired mass was detected. The mixture was concentrated under vacuum at 25° C. to give a residue which was purified by preparative HPLC (Phenomenex Luna C18 column (75×30 mm, 3 μm); flow rate: 25 mL/min; gradient: 1%-30% B over 8 min; mobile phase A: 0.1% aqueous TFA, mobile phase B: acetonitrile). (S)-2-Methyl-5-(pyrrolidin-2-ylmethoxy)-N-(1-(7-vinylquinolin-5-yl)cyclopropyl)benzamide (34.1 mg, 63.0 μmol, 72% yield, TFA salt) was obtained as a white solid. M+H⁺=428.4 (LCMS); ¹H NMR (400 MHZ, DMSO-d₆) δ 9.18-9.09 (m, 3H), 8.96 (dd, J=1.5, 4.4 Hz, 1H), 8.73-8.51 (m, 1H), 8.08 (d, J=1.5 Hz, 1H), 7.97 (s, 1H), 7.64 (dd, J=4.3, 8.5 Hz, 1H), 7.12-7.07 (m, 1H), 7.05-6.96 (m, 1H), 6.89 (dd, J=2.8, 8.4 Hz, 1H), 6.66 (d, J=2.8 Hz, 1H), 6.11 (d, J=17.6 Hz, 1H), 5.51 (d, J=11.0 Hz, 1H), 4.15 (dd, J=3.6, 10.6 Hz, 1H), 3.98 (dd, J=8.4, 10.5 Hz, 1H), 3.88-3.79 (m, 1H), 3.23-3.14 (m, 2H), 2.19-2.02 (m, 1H), 1.99-1.96 (m, 3H), 1.95-1.83 (m, 2H), 1.74-1.62 (m, 1H), 1.37 (br s, 2H), 1.29 (br s, 2H).

### Example 398: 2-Methyl-5-((tetrahydro-1H-pyrrolizin-7a (5H)-yl)methoxy)-N-(1-(7-vinylquinolin-5-yl)cyclopropyl)benzamide (Compound 748)

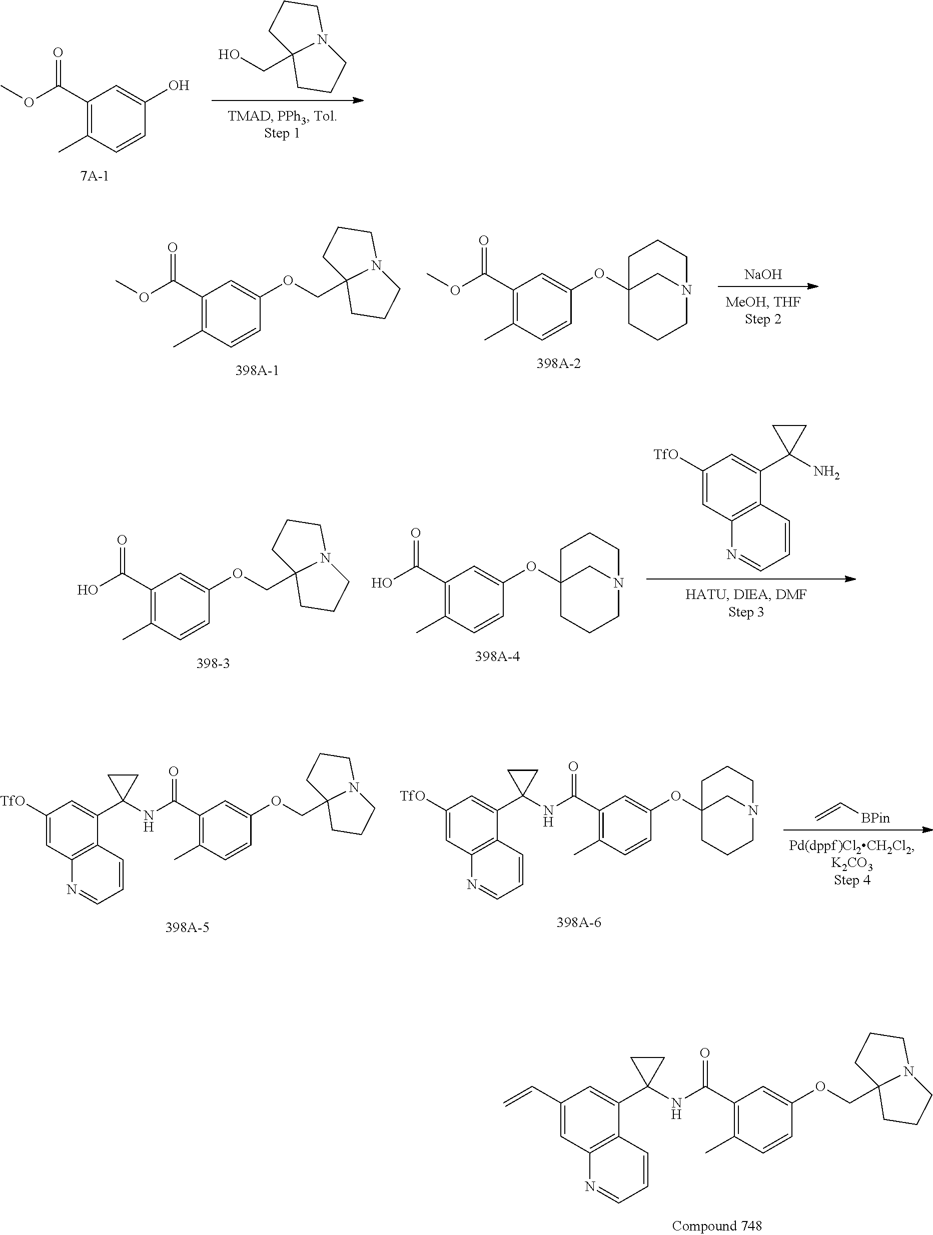

### Step 1: Methyl 2-methyl-5-((tetrahydro-1H-pyrrolizin-7a (5H)-yl)methoxy)benzoate (398A-1) and methyl 5-((1-azabicyclo[3.3.1]nonan-5-yl)oxy)-2-methylbenzoate (398A-2)

To a solution of methyl 5-hydroxy-2-methylbenzoate (2.00 g, 12.0 mmol, 1.0 eq) and (tetrahydro-1H-pyrrolizin-7a (5H)-yl)methanol (1.70 g, 12.0 mmol, 1.0 eq) in toluene (20 mL) was added TMAD (3.11 g, 18.0 mmol, 1.5 eq). The mixture was stirred at 100° C. under a N₂ atmosphere for 12 h. LCMS indicated that the starting material was completely consumed. The mixture was treated with water (40 mL) and extracted with EtOAc (40 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. Methyl 2-methyl-5-((tetrahydro-1H-pyrrolizin-7a (5H)-yl)methoxy)benzoate and methyl 5-((1-azabicyclo[3.3.1]nonan-5-yl)oxy)-2-methylbenzoate (3.50 g, 7.38 mmol, 61% yield) were obtained as a mixture of a white solid. M+H⁺=290.2 (LCMS).

### Step 2: 2-Methyl-5-((tetrahydro-1H-pyrrolizin-7a (5H)-yl)methoxy)benzoic acid (398A-3) and 5-((1-azabicyclo[3.3.1]nonan-5-yl)oxy)-2-methylbenzoic acid (398A-4)

To a mixture of methyl 2-methyl-5-((tetrahydro-1H-pyrrolizin-7a (5H)-yl)methoxy)benzoate and methyl 5-((1-azabicyclo[3.3.1]nonan-5-yl)oxy)-2-methylbenzoate (2.68 g, 5.56 mmol, 1.0 eq) in a mixture of MeOH (4.0 mL) and THF (12 mL) was added NaOH (11.1 mL, 2 M aqueous, 4.0 eq). The mixture was stirred at 70° C. for 12 h. LCMS indicated that the starting material was completely consumed. The mixture was concentrated under vacuum at 30° C. to give a residue which was purified by preparative HPLC (Phenomenex Gemini C18 column (75× 30 mm, 3 μm); flow rate: 25 mL/min; gradient: 1%-30% B over 8 min; mobile phase A: 0.1% aqueous TFA, mobile phase B: acetonitrile) to give 2-methyl-5-((tetrahydro-1H-pyrrolizin-7a (5H)-yl)methoxy)benzoic acid and 5-((1-azabicyclo[3.3.1]nonan-5-yl)oxy)-2-methylbenzoic acid (300 mg, 1.09 mmol, 19% yield) as a mixture of a white solid. M+H⁺=276.2 (LCMS).

### Step 3: 5-(1-(2-Methyl-5-((tetrahydro-1H-pyrrolizin-7a (5H)-yl)methoxy)benzamido)cyclopropyl)quinolin-7-yl trifluoromethanesulfonate (398A-5) and 5-(1-(5-((1-azabicyclo[3.3.1]nonan-5-yl)oxy)-2-methylbenzamido)cyclopropyl)quinolin-7-yl trifluoromethanesulfonate (398A-6)

To a mixture of 2-methyl-5-((tetrahydro-1H-pyrrolizin-7a (5H)-yl)methoxy)benzoic acid and 5-((1-azabicyclo[3.3.1]nonan-5-yl)oxy)-2-methylbenzoic acid (130 mg, 472 μmol, 1.0 eq) in DMF (2.0 mL) were added 5-(1-aminocyclopropyl)quinolin-7-yl trifluoromethanesulfonate (156 mg, 472 μmol, 1.0 eq), HATU (269 mg, 708 μmol, 1.5 eq) and DIEA (183 mg, 1.42 mmol, 247 μL, 3.0 eq). The resulting mixture was stirred at 20° C. for 2 h. LCMS indicated that the starting material was completely consumed. The mixture was concentrated under vacuum to give a residue which was purified by preparative HPLC (Phenomenex Gemini C18 column (100×30 mm, 10 μm); flow rate: 60 mL/min; gradient: 40%-70% B over 8 min; mobile phase A: 0.1% aqueous NH₄HCO₃, mobile phase B: acetonitrile). 5-(1-(2-Methyl-5-((tetrahydro-1H-pyrrolizin-7a (5H-yl)methoxy)benzamido)cyclopropyl)quinolin-7-yl trifluoromethanesulfonate (25.0 mg, 38.5 μmol, 8% yield) was obtained as a white solid. M+H⁺=590.2 (LCMS). 5-(1-(5-((1-Azabicyclo[3.3.1]nonan-5-yl)oxy)-2-methylbenzamido)cyclopropyl)quinolin-7-yl trifluoromethanesulfonate (30.0 mg, 38.5 μmol, 10% yield) was obtained as a white solid. M+H⁺=590.2 (LCMS).

### Step 4: 2-Methyl-5-((tetrahydro-1H-pyrrolizin-7a (5H)-yl)methoxy)-N-(1-(7-vinylquinolin-5-yl)cyclopropyl)benzamide (Compound 748)

To a solution of 5-(1-(2-methyl-5-((tetrahydro-1H-pyrrolizin-7a (5H-yl)methoxy)benzamido)cyclopropyl)quinolin-7-yl trifluoromethanesulfonate (25.0 mg, 42.4 μmol, 1.0 eq) and 4,4,5,5-tetramethyl-2-vinyl-1,3,2-dioxaborolane (13.0 mg, 84.8 μmol, 2.0 eq) in a mixture of dioxane (1.0 mL) and H₂O (100 μL) were added Na₂CO₃ (10.3 mg, 97.5 μmol, 2.3 eq) and Pd(dppf)Cl₂·CH₂Cl₂ (3.46 mg, 4.24 μmol, 0.10 eq). The resulting mixture was stirred at 80° C. for 12 h under a N₂ atmosphere. LCMS indicated that the starting material was completely consumed, and the desired mass was detected. The mixture was concentrated under vacuum at 30° C. to give a residue which was purified by preparative HPLC (Phenomenex Gemini C18 column (80×30 mm, 3 μm); flow rate: 25 mL/min; gradient: 1%-20% B over 8 min; mobile phase A: 0.04% aqueous HCl, mobile phase B: acetonitrile) to give 2-methyl-5-((tetrahydro-1H-pyrrolizin-7a (5H)-yl)methoxy)-N-(1-(7-vinylquinolin-5-yl)cyclopropyl)benzamide (10.9 mg, 21.6 μmol, 51% yield, HCl salt) as a white solid. ¹H NMR (400 MHZ, DMSO-d₆) δ 10.32-10.11 (m, 1H), 9.32 (td, J=2.6, 4.9 Hz, 1H), 9.20 (s, 1H), 9.05 (br d, J=4.0 Hz, 1H), 8.15 (s, 1H), 8.04 (s, 1H), 7.77 (br d, J=9.4 Hz, 1H), 7.12-6.99 (m, 2H), 6.90 (dd, J=2.7, 8.1 Hz, 1H), 6.72 (d, J=2.4 Hz, 1H), 6.18 (s, 1H), 5.57 (d, J=10.7 Hz, 1H), 4.07 (s, 2H), 3.47-3.41 (m, 2H), 3.21-3.13 (m, 2H), 2.11-2.03 (m, 4H), 1.98-1.91 (m, 7H), 1.40 (br s, 2H), 1.32 (br s, 2H).

### Example 399: 5-(1-Azabicyclo[3.3.1]nonan-5-yloxy)-2-methyl-N-(1-(7-vinylquinolin-5-yl)cyclopropyl)benzamide (Compound 749)

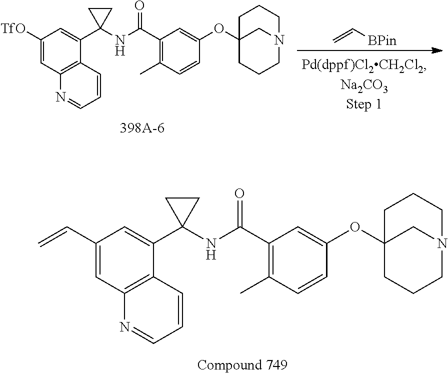

### Step 1: 5-((1-Azabicyclo[3.3.1]nonan-5-yl)oxy)-2-methyl-N-(1-(7-vinylquinolin-5-yl)cyclopropyl)benzamide (Compound 749)

To a solution of 5-(1-(5-((1-azabicyclo[3.3.1]nonan-5-yl)oxy)-2-methylbenzamido)cyclopropyl)quinolin-7-yl trifluoromethanesulfonate (19.5 mg, 33.1 μmol, 1.0 eq) and 4,4,5,5-tetramethyl-2-vinyl-1,3,2-dioxaborolane (10.2 mg, 66.2 μmol, 2.0 eq) in a mixture of dioxane (1.0 mL) and H₂O (100 μL) were added Na₂CO₃ (8.00 mg, 76.2 μmol, 2.3 eq) and Pd(dppf)Cl₂·CH₂Cl₂ (2.71 mg, 3.31 μmol, 0.10 eq). The resulting mixture was stirred at 80° C. for 12 h under a N₂ atmosphere. LCMS indicated that the starting material was completely consumed, and the desired mass was detected. The mixture was concentrated under vacuum at 30° C. to give a residue which was purified by preparative HPLC (Phenomenex Gemini C18 column (80×30 mm, 3 μm); flow rate: 25 mL/min; gradient: 1%-25% B over 8 min; mobile phase A: 0.04% aqueous HCl, mobile phase B: acetonitrile) to give 5-((1-azabicyclo[3.3.1]nonan-5-yl)oxy)-2-methyl-N-(1-(7-vinylquinolin-5-yl)cyclopropyl)benzamide (5.80 mg, 11.4 μmol, 34% yield, HCl salt) as a white solid. ¹H NMR (400 MHZ, DMSO-d₆) δ 10.68 (br s, 1H), 9.36-9.17 (m, 2H), 9.10-8.98 (m, 1H), 8.12 (s, 1H), 8.02 (s, 1H), 7.77 (br dd, J=3.1, 4.7 Hz, 1H), 7.09 (d, J=8.4 Hz, 1H), 7.03 (dd, J=10.9, 17.7 Hz, 1H), 6.92 (dd, J=2.6, 8.2 Hz, 1H), 6.67 (d, J=2.5 Hz, 1H), 6.15 (d, J=17.4 Hz, 1H), 5.56 (d, J=11.0 Hz, 1H), 3.23-3.11 (m, 6H), 2.24-2.11 (m, 2H), 2.02-1.99 (m, 3H), 1.99-1.93 (m, 2H), 1.87-1.77 (m, 4H), 1.40 (br s, 2H), 1.31 (br s, 2H).

### Example 400: (S)-4-Fluoro-2-methyl-5-((1-methylazetidin-2-yl)methoxy)-N-(1-(7-vinylquinolin-5-yl)cyclopropyl)benzamide (Compound 800)

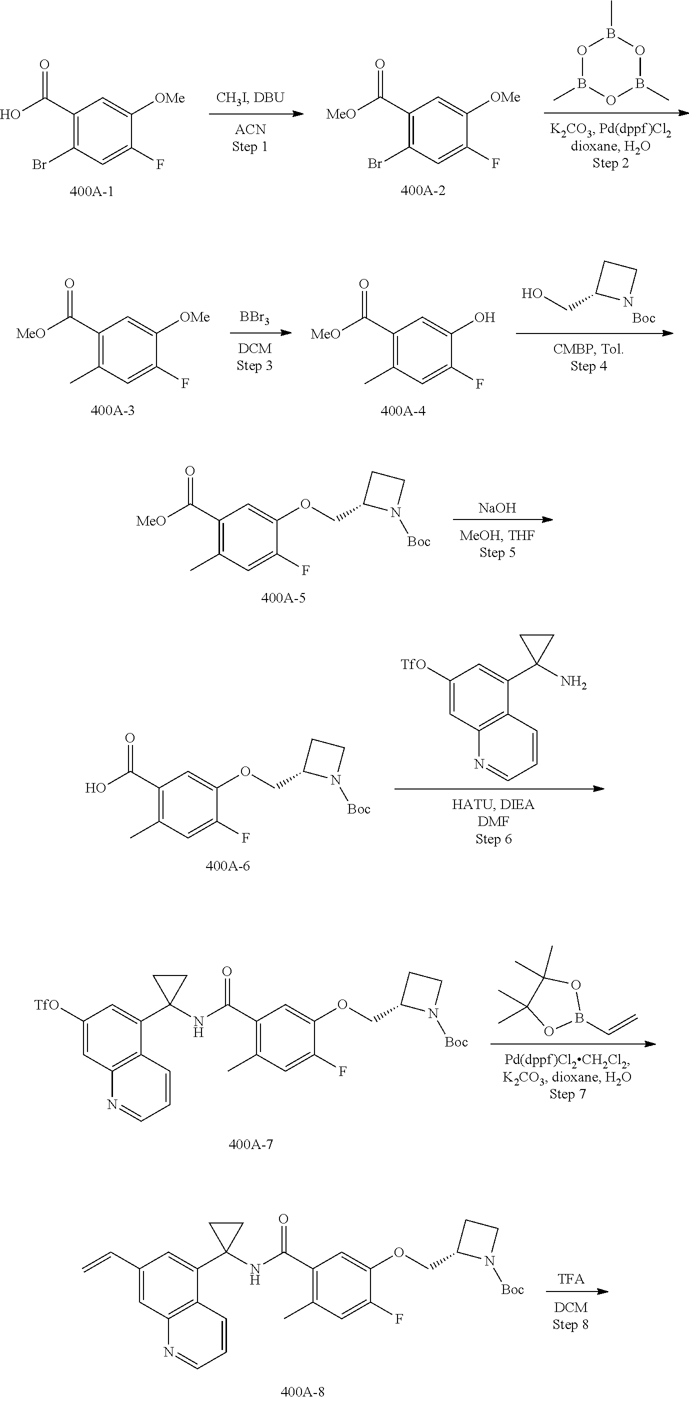

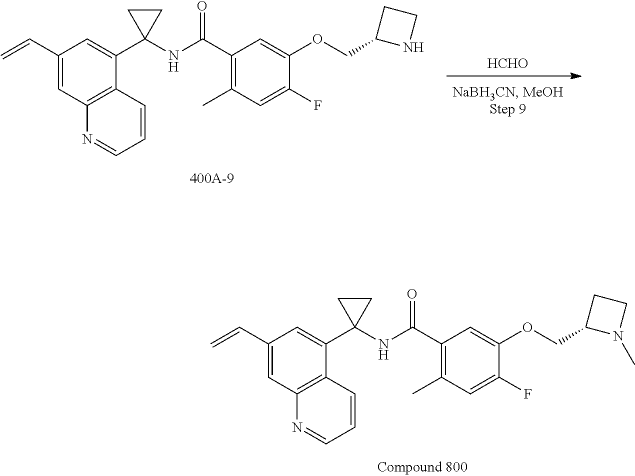 [SMILES: C=Cc1cc(C2(NC(=O)c3cc(OC[C@@H]4CCN4)c(F)cc3C)CC2)c2cccnc2c1 ; "HCHO" ; C=Cc1cc(C2(NC(=O)c3cc(OC[C@@H]4CCN4C)c(F)cc3C)CC2)c2cccnc2c1]

### Step 1: Methyl 2-bromo-4-fluoro-5-methoxybenzoate (400A-2)

To a solution of 2-bromo-4-fluoro-5-methoxybenzoic acid (1.20 g, 4.82 mmol, 1.0 eq) in acetonitrile (30 mL) were added CH₃I (1.73 g, 9.64 mmol, 600 μL, 2.0 eq) and DBU (1.25 g, 8.19 mmol, 1.23 mL 1.7 eq). The mixture was stirred at 20° C. for 16 h. LCMS indicated that the starting material was completely consumed, and the desired mass was detected. The reaction mixture was poured into H₂O (30 mL) and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 1/100 to 1/10. Methyl 2-bromo-4-fluoro-5-methoxybenzoate (1.25 g, 4.75 mmol, 99% yield) was obtained as a white solid. M+H⁺=263.1 (LCMS).

### Step 2: Methyl 4-fluoro-5-methoxy-2-methylbenzoate (400A-3)

A solution of methyl 2-bromo-4-fluoro-5-methoxybenzoate (1.25 g, 4.76 mmol, 1.0 eq) in a mixture of dioxane (60 mL) and H₂O (12 mL) was degassed and purged with N₂ three times. To this mixture were added 2,4,6-trimethyl-1,3,5,2,4,6-trioxatriborinane (2.38 g, 9.50 mmol, 2.66 mL, 50% purity, 2.0 eq), K₂CO₃ (1.97 g, 14.3 mmol, 3.0 eq), and Pd (dppf) C12 (348 mg, 2.53 mmol, 475 L, 0.1 eq). The mixture was stirred at 80° C. for 16 h under a N₂ atmosphere. TLC indicated that the starting material was completely consumed. The reaction mixture was allowed to cool to room temperature, poured into H₂O (60 mL), and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 1/100 to 1/10. Methyl 4-fluoro-5-methoxy-2-methylbenzoate (722 mg, 3.64 mmol, 77% yield) was obtained as a white solid.

### Step 3: Methyl 4-fluoro-5-hydroxy-2-methylbenzoate (400A-4)

To a solution of methyl 4-fluoro-5-methoxy-2-methylbenzoate (722 mg, 3.64 mmol, 1.0 eq) in DCM (40 mL) was added BBr₃ (13.7 g, 54.6 mmol, 6.92 mL, 15 eq). The mixture was stirred at −78° C. for 1 h under a N₂ atmosphere, then at 20° C. for 16 h under a N₂ atmosphere. LCMS indicated that the starting material was completely consumed, and the desired mass was detected. The mixture was concentrated under vacuum at 20° C. to give a residue. The reaction mixture was poured into MeOH (2.0 ml) and basified to pH 8 with NH₃·H₂O. The mixture was concentrated under vacuum to give a residue which was purified by flash silica gel chromatography using a gradient of EtOAc/petroleum ether from 1/100 to 3/5. Methyl 4-fluoro-5-hydroxy-2-methylbenzoate (500 mg, 2.71 mol, 75% yield) was obtained as a yellow solid. M+H⁺=185.2 (LCMS).

### Step 4: tert-Butyl(S)-2-((2-fluoro-5-(methoxycarbonyl)-4-methylphenoxy)methyl)azetidine-1-carboxylate (400A-5)

To a solution of methyl 4-fluoro-5-hydroxy-2-methylbenzoate (490 mg, 2.66 mmol, 1.0 eq) and tert-butyl(S)-2-(hydroxymethyl)azetidine-1-carboxylate (498 mg, 2.66 mmol, 1.0 eq) in toluene (30 mL) were added TMAD (1.37 g, 7.98 mmol, 3.0 eq) and PPh₃ (2.09 g, 7.98 mmol, 3.0 eq). The mixture was stirred at 100° C. for 16 h under a N₂ atmosphere. LCMS indicated that the starting material was completely consumed, and the desired mass was detected. The reaction mixture was allowed to cool to room temperature and poured into H₂O (30 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/100 to 3/5. tert-Butyl (S)-2-((2-fluoro-5-(methoxycarbonyl)-4-methylphenoxy)methyl)azetidine-1-carboxylate (820 mg, 2.32 mmol, 87% yield) was obtained as a white oil. M−56+H⁺==298.2 (LCMS).

### Step 5: (S)-5-((1-(tert-Butoxycarbonyl)azetidin-2-yl)methoxy)-4-fluoro-2-methylbenzoic acid (400A-6)

To a solution of tert-butyl(S)-2-((2-fluoro-5-(methoxycarbonyl)-4-methylphenoxy)methyl) azetidine-1-carboxylate (820 mg, 2.32 mmol, 1.0 eq) in a mixture of MeOH (36 mL) and THF (18 mL) was added NaOH (2 M aqueous, 4.6 mL, 4.0 eq). The mixture was stirred at 70° C. for 16 h. LCMS indicated that the starting material was completely consumed, and the desired mass was detected. The reaction mixture was allowed to cool to room temperature, poured into H₂O (30 mL), and washed with MTBE (15 mL×2). The aqueous layer was acidified to pH 6 with HCl (1 M aqueous) and extracted with EtOAc (8.0 mL×3). The combined organic layers were dried over Na₂SO₄, filtered, and concentrated under vacuum to give the crude(S)-5-((1-(tert-butoxycarbonyl)azetidin-2-yl)methoxy)-4-fluoro-2-methylbenzoic acid (787 mg, 2.32 mmol, 100% yield) as a brown oil. M−56+H⁺=284.1 (LCMS).

### Step 6: tert-Butyl(S)-2-((2-fluoro-4-methyl-5-((1-(7-(((trifluoromethyl)sulfonyl)oxy)quinolin-5-yl)cyclopropyl)carbamoyl)phenoxy)methyl)azetidine-1-carboxylate (400A-7)

To a solution of(S)-5-((1-(tert-butoxycarbonyl)azetidin-2-yl)methoxy)-4-fluoro-2-methylbenzoic acid (306 mg, 903 μmol, 1.0 eq) and 5-(1-aminocyclopropyl)quinolin-7-yl trifluoromethanesulfonate (300 mg, 903 μmol, 1.0 eq) in DMF (30 mL) were added DIEA (350 mg, 2.71 mmol, 472 μL, 3.0 eq) and HATU (858 mg, 2.26 mmol, 2.5 eq). The mixture was stirred at 20° C. for 16 h. LCMS indicated that the starting material was completely consumed, and the desired mass was detected. The reaction mixture was poured into H₂O (30 mL) and extracted with EtOAc (12 mL×3). The combined organic layers were dried over Na₂SO₄, filtered, and concentrated under vacuum to give a residue which was purified by flash silica gel chromatography using a gradient of EtOAc/petroleum ether from 1/100 to 3/5. tert-Butyl (S)-2-((2-fluoro-4-methyl-5-((1-(7-(((trifluoromethyl)sulfonyl)oxy)quinolin-5-yl)cyclopropyl)carbamoyl)phenoxy)methyl)azetidine-1-carboxylate (571 mg, 874 μmol, 97% yield) was obtained as a brown oil. M−56+H⁺=598.2 (LCMS).

### Step 7: tert-Butyl(S)-2-((2-fluoro-4-methyl-5-((1-(7-vinylquinolin-5-yl)cyclopropyl)carbamoyl)phenoxy)methyl)azetidine-1-carboxylate (400A-8)

To a solution of tert-butyl(S)-2-((2-fluoro-4-methyl-5-((1-(7-(((trifluoromethyl)sulfonyl) oxy)quinolin-5-yl)cyclopropyl)carbamoyl)phenoxy)methyl)azetidine-1-carboxylate (571 mg, 874 μmol, 184 μL 1.0 eq) and 4,4,5,5-tetramethyl-2-vinyl-1,3,2-dioxaborolane (135 mg, 874 μmol, 1.0 eq) in a mixture of dioxane (30 mL) and H₂O (6.0 mL) were added Pd(dppf)Cl₂·CH₂Cl₂ (71.3 mg, 87.3 μmol, 0.1 eq) and Na₂CO₃ (213 mg, 2.01 mmol, 2.3 eq). The mixture was degassed and purged with N₂ three times. The mixture was stirred at 80° C. for 16 h under a N₂ atmosphere. LCMS indicated that the starting material was completely consumed, and the desired mass was detected. The reaction mixture was allowed to cool to room temperature, poured into H₂O (30 mL), and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 1/100 to 3/5. tert-Butyl(S)-2-((2-fluoro-4-methyl-5-((1-(7-vinylquinolin-5-yl)cyclopropyl)carbamoyl)phenoxy)methyl)azetidine-1-carboxylate (280 mg, 527 μmol, 60% yield) was obtained as a yellow oil. M−56+H⁺=476.4 (LCMS).

### Step 8: (S)-5-(Azetidin-2-ylmethoxy)-4-fluoro-2-methyl-N-(1-(7-vinylquinolin-5-yl)cyclo propyl)benzamide (400A-9)

To a solution of tert-butyl(S)-2-((2-fluoro-4-methyl-5-((1-(7-vinylquinolin-5-yl)cyclopropyl) carbamoyl)phenoxy)methyl)azetidine-1-carboxylate (280 mg, 527 μmol, 1.0 eq) in DCM (10 mL) was added TFA (4.93 g, 43.3 mmol, 3.2 mL, 82 eq). The mixture was stirred at 20° C. for 1 h. LCMS indicated that the starting material was completely consumed, and the desired mass was detected. The reaction mixture was concentrated under vacuum to give the crude(S)-5-(azetidin-2-ylmethoxy)-4-fluoro-2-methyl-N-(1-(7-vinylquinolin-5-yl)cyclopropyl)benzamide (500 mg) as a yellow oil. M+H⁺=432.3 (LCMS).

### Step 9: (S)-4-Fluoro-2-methyl-5-((1-methylazetidin-2-yl)methoxy)-N-(1-(7-vinylquinolin-5-yl)cyclopropyl)benzamide (Compound 800)

To a solution of(S)-5-(azetidin-2-ylmethoxy)-4-fluoro-2-methyl-N-(1-(7-vinylquinolin-5-yl)cyclopropyl)benzamide (500 mg, 1.07 mmol, 1.0 eq, TFA salt) in MeOH (10 mL) was added TEA (0.1 μL), followed by formaldehyde (173 mg, 2.14 mmol, 159 μL, 37% purity in water, 2.0 eq). The resulting mixture was adjusted to pH 6 with a small amount of AcOH, then NaBH₃CN (134 mg, 2.14 mmol, 2.0 eq) was added. The mixture was stirred at 20° C. for 16 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by preparative HPLC (column: Phenomenex Luna (80×30 mm, 3 μm); flow rate: 60 mL/min; gradient: 1%-25% B over 8 min; mobile phase A: 0.04% aqueous HCl, mobile phase B: acetonitrile). (S)-4-Fluoro-2-methyl-5-((1-methylazetidin-2-yl)methoxy)-N-(1-(7-vinylquinolin-5-yl)cyclopropyl)benzamide (54.0 mg, 107 μmol, 10% yield, HCl salt) was obtained as a yellow solid. M+H⁺=446.4 (LCMS); ¹H NMR (400 MHZ, DMSO-d₆) δ 10.78 (br d, J=3.3 Hz, 1H), 9.56-9.42 (m, 1H), 9.39-9.28 (m, 1H), 9.11 (br d, J=4.5 Hz, 1H), 8.20 (s, 1H), 8.09 (s, 1H), 7.95-7.80 (m, 1H), 7.16-6.92 (m, 3H), 6.19 (d, J=17.6 Hz, 1H), 5.61 (d, J=11.0 Hz, 1H), 4.70-4.63 (m, 1H), 4.45-4.32 (m, 2H), 4.01-3.98 (m, 1H), 3.89-3.85 (m, 1H), 2.81 (d, J=4.9 Hz, 3H), 2.42-2.23 (m, 2H), 1.99 (s, 3H), 1.43 (br s, 2H), 1.34 (br s, 2H).

### Example 401: (S)-4-Amino-2-methyl-5-((1-methylazetidin-2-yl)methoxy)-N-(1-(7-vinylquinolin-5-yl)cyclopropyl)benzamide (Compound 726)

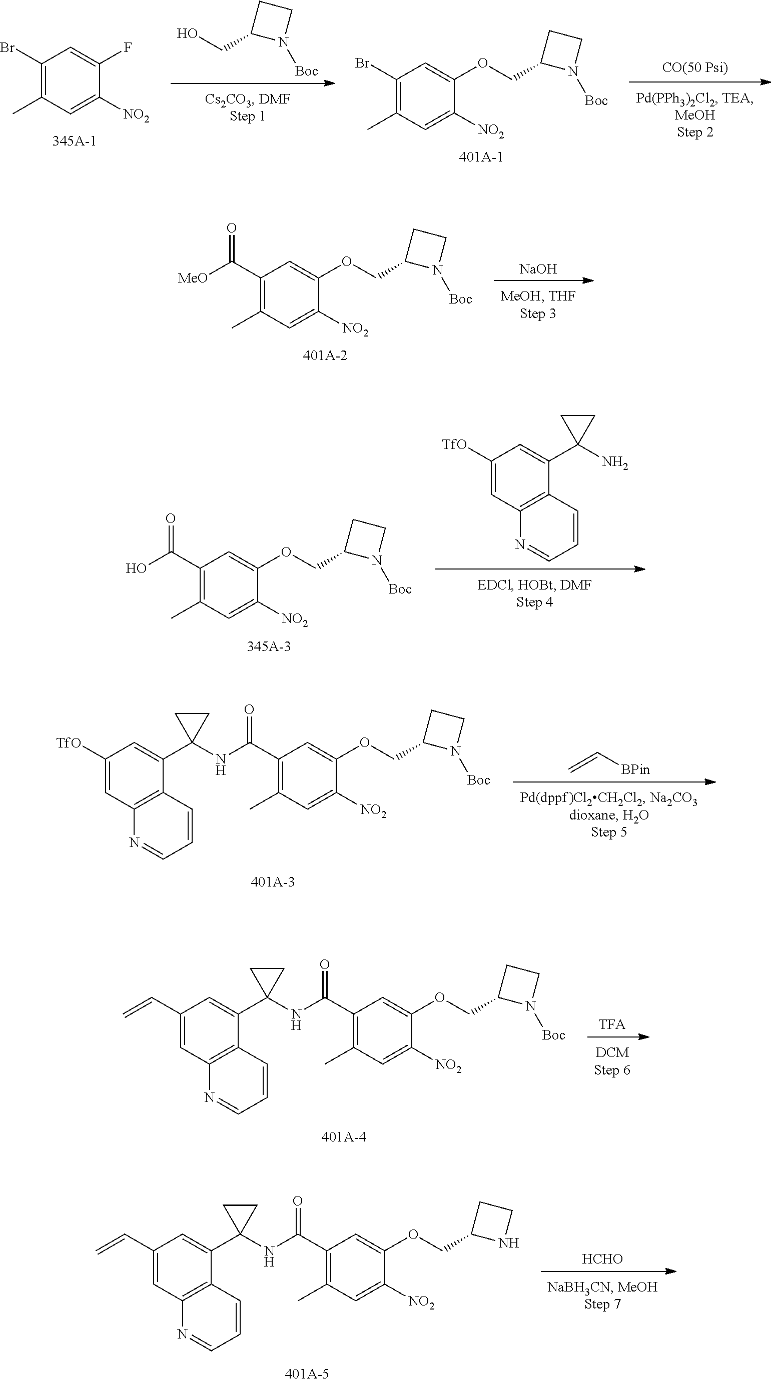

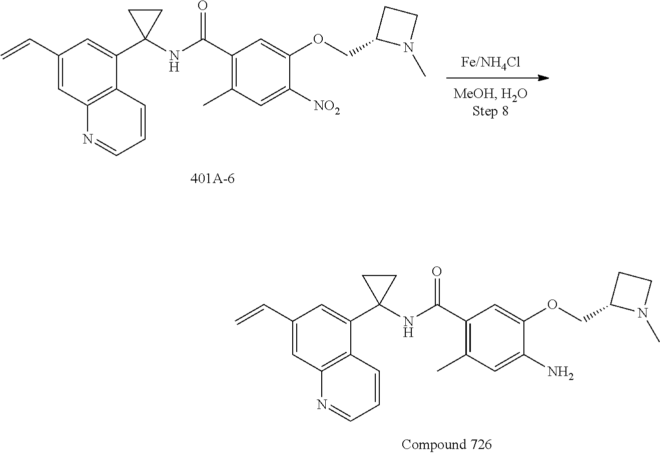

### Step 1: tert-Butyl(S)-2-((5-bromo-4-methyl-2-nitrophenoxy)methyl)azetidine-1-carboxylate (401A-1)

To a solution of 1-bromo-5-fluoro-2-methyl-4-nitrobenzene (500 mg, 2.14 mmol, 1.0 eq) in DMF (20 mL) were added Cs₂CO₃ (766 mg, 2.35 mmol, 1.1 eq) and tert-butyl(S)-2-(hydroxymethyl)azetidine-1-carboxylate (400 mg, 2.14 mmol, 1.0 eq). The resulting mixture was stirred at 60° C. under a N₂ atmosphere for 16 h. LCMS indicated that the starting material was completely consumed. The reaction mixture was allowed to cool to room temperature. The reaction mixture was poured into H₂O (15 mL) and extracted with EtOAc (6.0 mL×3). The combined organic layers were dried over Na₂SO₄, filtered, and concentrated under vacuum to give a residue which was purified by flash silica gel chromatography using a gradient of EtOAc/petroleum ether from 1/100 to 1/5. tert-Butyl(S)-2-((5-bromo-4-methyl-2-nitrophenoxy)methyl)azetidine-1-carboxylate (760 mg, 1.89 mmol, 89% yield) was obtained as a yellow solid. M−56+H⁺=345.1 (LCMS).

### Step 2: tert-Butyl(S)-2-((5-(methoxycarbonyl)-4-methyl-2-nitrophenoxy)methyl) azetidine-1-carboxylate (401A-2)

To a solution of tert-butyl(S)-2-((5-bromo-4-methyl-2-nitrophenoxy)methyl)azetidine-1-carboxylate (600 mg, 1.50 mmol, 1.0 eq) in MeOH (50 mL) were added Pd(PPh₃)₂Cl₂ (105 mg, 150 μmol, 0.1 eq) and TEA (6.05 g, 59.8 mmol, 8.33 mL, 40 eq). The mixture was degassed and purged with CO three times, then stirred at 70° C. for 16 h under a CO (50 psi) atmosphere. LCMS indicated that the starting material was completely consumed. The reaction mixture was allowed to cool to room temperature and concentrated under vacuum to give a residue which was purified by flash silica gel chromatography using a gradient of EtOAc/petroleum ether from 1/100 to 3/5 tert-Butyl(S)-2-((5-(methoxycarbonyl)-4-methyl-2-nitrophenoxy)methyl)azetidine-1-carboxylate (164 mg, 431 μmol, 14% yield) was obtained as a yellow oil. M−56+H⁺=325.2 (LCMS).

### Step 3: (S)-5-((1-(tert-Butoxycarbonyl)azetidin-2-yl)methoxy)-2-methyl-4-nitrobenzoic acid (345A-3)

To a solution of tert-butyl(S)-2-((5-(methoxycarbonyl)-4-methyl-2-nitrophenoxy)methyl) azetidine-1-carboxylate (164 mg, 431 μmol, 1.0 eq) in MeOH (11 mL) and THF (5.5 mL) was added NaOH (2 M aqueous, 1.0 mL, 4.0 eq). The mixture was stirred at 70° C. for 16 h. LCMS indicated that the starting material was completely consumed. The reaction mixture was allowed to cool to room temperature, poured into H₂O (10 mL), and washed with MTBE (5.0 mL×2). The aqueous layer was acidified to pH 6 with HCl (1 M aqueous). The product was extracted with EtOAc (5.0 mL×3). The combined organic layers were dried over Na₂SO₄, filtered, and concentrated under vacuum to give the product(S)-5-((1-(tert-butoxycarbonyl)azetidin-2-yl)methoxy)-2-methyl-4-nitrobenzoic acid (110 mg, 300 μmol, 70% yield) as a brown oil. M−56+H⁺=311.2 (LCMS).

### Step 4: tert-Butyl(S)-2-((4-methyl-2-nitro-5-((1-(7-(((trifluoromethyl)sulfonyl)oxy)quinolin-5-yl)cyclopropyl)carbamoyl)phenoxy)methyl)azetidine-1-carboxylate (401A-3)

To a solution of(S)-5-((1-(tert-butoxycarbonyl)azetidin-2-yl)methoxy)-2-methyl-4-nitrobenzoic acid (90.0 mg, 246 μmol, 1.0 eq) and 5-(1-aminocyclopropyl)quinolin-7-yl trifluoromethanesulfonate (73.5 mg, 221 μmol, 0.9 eq) in DMF (6.0 mL) were added EDCI (70.6 mg, 369 μmol, 1.5 eq), HOBt (49.8 mg, 369 μmol, 1.5 eq) and TEA (49.7 mg, 491 μmol, 68.4 μL, 2.0 eq). The mixture was stirred at 20° C. for 16 h. LCMS indicated that the starting material was completely consumed. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative TLC (EtOAc/petroleum ether=1/1, R_{f}=0.46). The crude product tert-butyl(S)-2-((4-methyl-2-nitro-5-((1-(7-(((trifluoromethyl)sulfonyl)oxy)quinolin-5-yl)cyclopropyl)carbamoyl) phenoxy)methyl)azetidine-1-carboxylate (180 mg) was obtained as a yellow oil. M−56+H⁺=625.3 (LCMS).

### Step 5: tert-Butyl(S)-2-((4-methyl-2-nitro-5-((1-(7-vinylquinolin-5-yl)cyclopropyl) carbamoyl)phenoxy)methyl)azetidine-1-carboxylate (401A-4)

To a solution of tert-butyl(S)-2-((4-methyl-2-nitro-5-((1-(7-(((trifluoromethyl)sulfonyl)oxy) quinolin-5-yl)cyclopropyl)carbamoyl)phenoxy)methyl)azetidine-1-carboxylate (170 mg, 250 μmol, 1.0 eq) and 4,4,5,5-tetramethyl-2-vinyl-1,3,2-dioxaborolane (38.5 mg, 250 μmol, 42.4 μL, 1.0 eq) in a mixture of dioxane (8.5 mL) and H₂O (1.7 mL) were added Pd(dppf)Cl₂·CH₂Cl₂ (20.4 mg, 25.0 μmol, 0.1 eq) and Na₂CO₃ (60.9 mg, 574 μmol, 2.3 eq). The resulting mixture was stirred at 80° C. under a N₂ atmosphere for 16 h. LCMS indicated that the starting material was completely consumed. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by preparative TLC (EtOAc/petroleum ether=1/0, R_{f}=0.23). tert-Butyl(S)-2-((4-methyl-2-nitro-5-((1-(7-vinylquinolin-5-yl)cyclopropyl)carbamoyl)phenoxy)methyl)azetidine-1-carboxylate (115 mg, 206 μmol, 82% yield) was obtained as a yellow solid. M+H⁺=559.4 (LCMS).

### Step 6: (S)-5-(Azetidin-2-ylmethoxy)-2-methyl-4-nitro-N-(1-(7-vinylquinolin-5-yl)cyclopropyl)benzamide (401A-5)

To a solution of tert-butyl(S)-2-((4-methyl-2-nitro-5-((1-(7-vinylquinolin-5-yl)cyclopropyl) carbamoyl)phenoxy)methyl)azetidine-1-carboxylate (115 mg, 206 μmol, 1.0 eq) in DCM (5.0 mL) was added TFA (1.93 g, 16.9 mmol, 1.3 mL, 82 eq). The mixture was stirred at 20° C. for 1 h. LCMS indicated that the starting material was completely consumed. The reaction mixture was concentrated under vacuum to give the crude(S)-5-(azetidin-2-ylmethoxy)-2-methyl-4-nitro-N-(1-(7-vinylquinolin-5-yl)cyclopropyl)benzamide (240 mg, TFA salt) as a yellow solid. M+H⁺=459.3 (LCMS).

### Step 7: (S)-2-Methyl-5-((1-methylazetidin-2-yl)methoxy)-4-nitro-N-(1-(7-vinylquinolin-5-yl)cyclopropyl)benzamide (401A-6)

To a solution of(S)-5-(azetidin-2-ylmethoxy)-2-methyl-4-nitro-N-(1-(7-vinylquinolin-5-yl)cyclopropyl)benzamide (240 mg, 419 μmol, 1.0 eq, TFA salt) in MeOH (12 mL) was added TEA (2.4 mL), followed by formaldehyde (68.0 mg, 838 μmol, 62.4 μL, 37% purity in water, 2.0 eq). The resulting mixture was adjusted to pH 6 with a small amount of AcOH. Then NaBH₃CN (52.7 mg, 838 μmol, 2.0 eq) was added. The mixture was stirred at 20° C. for 16 h. LCMS indicated that the starting material was completely consumed. The reaction mixture was poured into H₂O (15 mL) and extracted with EtOAc (10 mL×3). The combined organic layers were dried over Na₂SO₄, filtered, and concentrated under vacuum to give a residue which was purified by preparative TLC (DCM/MeOH=10/1, R_{f}=0.21). (S)-2-Methyl-5-((1-methylazetidin-2-yl)methoxy)-4-nitro-N-(1-(7-vinylquinolin-5-yl)cyclopropyl)benzamide (50.0 mg, 106 μmol, 25% yield) was obtained as a yellow oil. M+H⁺=473.3 (LCMS).

### Step 8: (S)-4-Amino-2-methyl-5-((1-methylazetidin-2-yl)methoxy)-N-(1-(7-vinylquinolin-5-yl)cyclopropyl)benzamide (Compound 726)

To a solution of(S)-2-methyl-5-((1-methylazetidin-2-yl)methoxy)-4-nitro-N-(1-(7-vinylquinolin-5-yl)cyclopropyl)benzamide (50.0 mg, 106 μmol, 1.0 eq) in MeOH (2.5 mL) and H₂O (0.5 mL) were added NH₄Cl (28.3 mg, 529 μmol, 5.0 eq) and iron powder (29.6 mg, 529 μmol, 5.0 eq). The mixture was stirred at 80° C. for 2 h. LCMS indicated that the starting material was completely consumed. The reaction mixture was allowed to cool to room temperature, poured into H₂O (7.0 mL), and extracted with EtOAc (5.0 mL×3). The combined organic layers were dried over Na₂SO₄, filtered, and concentrated under vacuum to give a residue which was purified by preparative HPLC (column: Phenomenex Luna C18 (100×30 mm, 5 μm); flow rate: 60 mL/min; gradient: 1%-23% B over 8 min; mobile phase A: 0.1% aqueous TFA, mobile phase B: acetonitrile). (S)-4-Amino-2-methyl-5-((1-methylazetidin-2-yl)methoxy)-N-(1-(7-vinylquinolin-5-yl)cyclopropyl)benzamide (12.6 mg, 22.5 μmol, 21% yield, TFA salt) was obtained as a white solid. M+H⁺=443.3 (LCMS); ¹H NMR (400 MHZ, DMSO-d₆) δ 9.16 (br d, J=8.7 Hz, 1H), 9.00-8.88 (m, 1H), 8.74 (s, 1H), 8.07 (s, 1H), 7.94 (s, 1H), 7.62 (dd, J=4.4, 8.4 Hz, 1H), 7.00 (dd, J=10.9, 17.7 Hz, 1H), 6.66 (s, 1H), 6.41 (s, 1H), 6.09 (d, J=17.2 Hz, 1H), 5.50 (d, J=10.9 Hz, 1H), 4.70-4.54 (m, 1H), 4.15 (br d, J=3.1 Hz, 1H), 4.10-4.01 (m, 1H), 3.92-3.82 (m, 2H), 2.87 (s, 3H), 2.39-2.30 (m, 2H), 2.00 (s, 3H), 1.35 (br s, 2H), 1.30-1.22 (m, 2H).

### Example 402: (S)-4-Acetamido-2-methyl-5-((1-methylazetidin-2-yl)methoxy)-N-(1-(7-vinylquinolin-5-yl)cyclopropyl)benzamide (Compound 746)

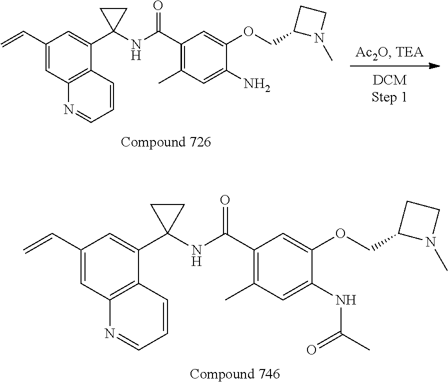

### Step 1: (S)-4-Acetamido-2-methyl-5-((1-methylazetidin-2-yl)methoxy)-N-(1-(7-vinylquinolin-5-yl)cyclopropyl)benzamide (Compound 746)

To a solution of(S)-4-amino-2-methyl-5-((1-methylazetidin-2-yl)methoxy)-N-(1-(7-vinyl quinolin-5-yl)cyclopropyl)benzamide (70.0 mg, 158 μmol, 1.0 eq) in DCM (5.0 mL) were added TEA (64.0 mg, 633 μmol, δ8.1 μL, 4.0 eq) and Ac₂O (32.3 mg, 316 μmol, 29.6 μL, 2.0 eq). The mixture was stirred at 20° C. for 3 h. LCMS indicated that the starting material was completely consumed. The mixture was treated with water (5.0 mL) and extracted with EtOAc (2.0 mL×3). The combined organic layers were dried over Na₂SO₄, filtered, and concentrated under vacuum to give a residue which was purified by preparative HPLC (column: Phenomenex Luna (80×30 mm, 3 μm); flow rate: 25 mL/min; gradient: 1%-20% B over 8 min; mobile phase A: 0.04% aqueous HCl, mobile phase B: acetonitrile). (S)-4-Acetamido-2-methyl-5-((1-methylazetidin-2-yl)methoxy)-N-(1-(7-vinylquinolin-5-yl)cyclopropyl)benzamide (8.60 mg, 16.5 μmol, 10% yield, HCl salt) was obtained as a yellow solid. M+H⁺=485.4 (LCMS); ¹H NMR (400 MHZ, DMSO-d₆) δ 10.73-10.51 (m, 1H), 9.44 (s, 1H), 9.38-9.28 (m, 1H), 9.23-9.03 (m, 2H), 8.15 (s, 1H), 8.02 (s, 1H), 7.78 (br dd, J=3.8, 8.0 Hz, 1H), 7.62 (s, 1H), 7.04 (dd, J=10.8, 17.6 Hz, 1H), 6.82 (s, 1H), 6.16 (d, J=17.7 Hz, 1H), 5.57 (d, J=10.8 Hz, 1H), 4.69-4.60 (m, 1H), 4.27 (d, J=4.4 Hz, 1H), 4.06-4.00 (m, 1H), 3.85 (br dd, J=6.7, 9.1 Hz, 2H), 2.83 (d, J=5.1 Hz, 3H), 2.42-2.33 (m, 2H), 2.15-2.06 (m, 3H), 1.99-1.95 (m, 3H), 1.44-1.37 (m, 2H), 1.31 (br s, 2H).

### Example 403: (S)-2-Methyl-5-((1-methylazetidin-2-yl)methoxy)-N-(1-(7-vinyl-1,2,3,4-tetrahydroquinolin-5-yl)cyclopropyl)benzamide (Compound 785)

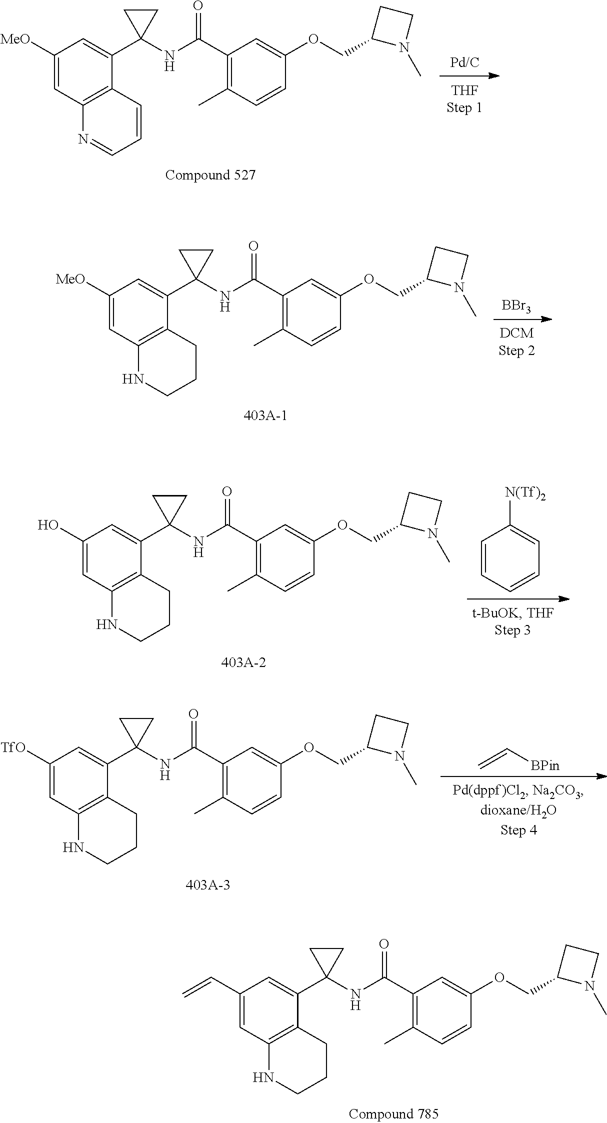

### Step 1: (S)—N-(1-(7-Methoxy-1,2,3,4-tetrahydroquinolin-5-yl)cyclopropyl)-2-methyl-5-((1-methylazetidin-2-yl)methoxy)benzamide (403A-1)

To a solution of(S)—N-(1-(7-methoxyquinolin-5-yl)cyclopropyl)-2-methyl-5-((1-methyl azetidin-2-yl)methoxy)benzamide (250 mg, 579 μmol, 1.0 eq) in THF (35 mL) was added 10% palladium on carbon (750 mg) under a N₂ atmosphere. The suspension was degassed and purged with H₂ three times. The mixture was stirred at 20° C. for 16 h under a H₂ (15 psi) atmosphere. LCMS indicated that the starting material completely consumed, and the desired product was detected. The suspension was filtered through a pad of Celite and the filter cake was washed with THF (10 mL×5). The combined filtrates were concentrated under vacuum to give(S)—N-(1-(7-methoxy-1,2,3,4-tetrahydroquinolin-5-yl)cyclopropyl)-2-methyl-5-((1-methylazetidin-2-yl)methoxy)benzamide (500 mg) as a white solid, which was used in the next step without any further purification. M+H⁺=436.1 (LCMS).

### Step 2: (S)—N-(1-(7-Hydroxy-1,2,3,4-tetrahydroquinolin-5-yl)cyclopropyl)-2-methyl-5-((1-methylazetidin-2-yl)methoxy)benzamide (403A-2)

To a solution of(S)—N-(1-(7-Methoxy-1,2,3,4-tetrahydroquinolin-5-yl)cyclopropyl)-2-methyl-5-((1-methylazetidin-2-yl)methoxy)benzamide (500 mg) in DCM (20 mL) was added BBr₃ (1.5 mL, 15 eq) dropwise at −78° C. under a N₂ atmosphere. The mixture was stirred at 25° C. for 3 h. LCMS indicated that the starting material was completely consumed, and the desired mass was detected. The mixture was concentrated under vacuum at 20° C. to give a residue. The residue was diluted with MeOH (5.0 mL) and adjusted to pH 8 with DIEA. The resulting mixture was concentrated under vacuum to give a residue which was purified by flash silica gel chromatography using a gradient of DCM/MeOH from 1/0 to 10/1. (S)—N-(1-(7-Hydroxy-1,2,3,4-tetrahydroquinolin-5-yl)cyclopropyl)-2-methyl-5-((1-methylazetidin-2-yl)methoxy)benzamide (270 mg, 640 μmol, 70% yield) was obtained as a yellow oil. M+H⁺=422.4 (LCMS).

### Step 3: (S)-5-(1-(2-Methyl-5-((1-methylazetidin-2-yl)methoxy)benzamido)cyclopropyl)-1,2,3,4-tetrahydroquinolin-7-yl trifluoromethanesulfonate (403A-3)

To a solution of(S)—N-(1-(7-hydroxy-1,2,3,4-tetrahydroquinolin-5-yl)cyclopropyl)-2-methyl-5-((1-methylazetidin-2-yl)methoxy)benzamide (200 mg, 474 μmol, 1.0 eq) in THF (7.0 mL) was added t-BuOK (53.0 mg, 474 μmol, 1 eq) at 0° C. The mixture was stirred at this temperature for 30 min. To this mixture was added 1,1,1-trifluoro-N-phenyl-N-((trifluoromethyl)sulfonyl)methanesulfonamide (170 mg, 474 μmol, 1.0 eq). Then the mixture was stirred at 25° C. for 16 h. LCMS indicated that the starting material was completely consumed, and the desired mass was detected. The reaction mixture was poured into H₂O (10 mL) and extracted with EtOAc (5.0 mL×2). The combined organic layers were dried over Na₂SO₄, filtered, and concentrated under vacuum to give a residue which was purified by preparative TLC (DCM/MeOH=10/1, R_{f}=0.7). (S)-5-(1-(2-Methyl-5-((1-methylazetidin-2-yl)methoxy)benzamido)cyclopropyl)-1,2,3,4-tetrahydroquinolin-7-yl trifluoromethanesulfonate (100 mg, 181 μmol, 38% yield) was obtained as a white solid. M+H⁺=554.2 (LCMS).

### Step 4: (S)-2-Methyl-5-((1-methylazetidin-2-yl)methoxy)-N-(1-(7-vinyl-1,2,3,4-tetra hydroquinolin-5-yl)cyclopropyl)benzamide (Compound 785)

To a solution of(S)-5-(1-(2-methyl-5-((1-methylazetidin-2-yl)methoxy)benzamido)cyclopropyl)-1,2,3,4-tetrahydroquinolin-7-yl trifluoromethanesulfonate (40.0 mg, 72.3 μmol, 1.0 eq) and 4,4,5,5-tetramethyl-2-vinyl-1,3,2-dioxaborolane (11.1 mg, 72.3 μmol, 12.3 μL, 1.0 eq) in a mixture of dioxane (1.0 mL) and H₂O (0.1 mL) were added Na₂CO₃ (23.0 mg, 217 μmol, 3.0 eq) and Pd(dppf)Cl₂·CH₂Cl₂ (5.90 mg, 7.23 μmol, 0.1 eq) under a N₂ atmosphere. The resulting mixture was stirred at 80° C. for 5 h under a N₂ atmosphere. LCMS indicated that the starting material was completely consumed, and the desired mass was detected. The reaction mixture was allowed to cool to room temperature, poured into H₂O (2.0 mL), and extracted with EtOAc (2.0 mL×2). The combined organic layers were dried over Na₂SO₄, filtered, and concentrated under vacuum to give a residue which was purified by preparative HPLC (Phenomenex Luna C18 column (100×40 mm, 5 μm); flow rate: 25 mL/min; gradient: 1%-30% B over 8 min; mobile phase A: 0.1% aqueous TFA, mobile phase B: acetonitrile). (S)-2-Methyl-5-((1-methylazetidin-2-yl)methoxy)-N-(1-(7-vinyl-1,2,3,4-tetrahydroquinolin-5-yl)cyclopropyl)benzamide (6.50 mg, 11.9 μmol, 17% yield, TFA salt) was obtained as a yellow solid. M+H⁺=432.4 (LCMS); ¹H NMR (400 MHZ, DMSO-d₆) δ 9.88 (br dd, J=3.8, 6.7 Hz, 1H), 8.71 (s, 1H), 7.13 (d, J=8.5 Hz, 1H), 6.99-6.91 (m, 2H), 6.78 (d, J=2.8 Hz, 1H), 6.61-6.49 (m, 2H), 5.60 (br d, J=17.4 Hz, 1H), 5.13 (br d, J=10.9 Hz, 1H), 4.69-4.58 (m, 1H), 4.28-4.22 (m, 2H), 4.04 (br dd, J=4.4, 9.4 Hz, 1H), 3.89-3.85 (m, 1H), 3.19 (br d, J=4.1 Hz, 2H), 2.93 (br t, J=6.1 Hz, 2H), 2.87 (d, J=4.9 Hz, 3H), 2.42-2.31 (m, 3H), 2.12 (s, 3H), 1.86-1.80 (m, 2H), 1.14-1.09 (m, 2H), 1.04 (br d, J=2.6 Hz, 2H).

### Example 404: (S)-2-Methyl-N-(1-(1-methyl-7-vinyl-1,2,3,4-tetrahydroquinolin-5-yl)cyclopropyl)-5-((1-methylazetidin-2-yl)methoxy)benzamide (Compound 796)

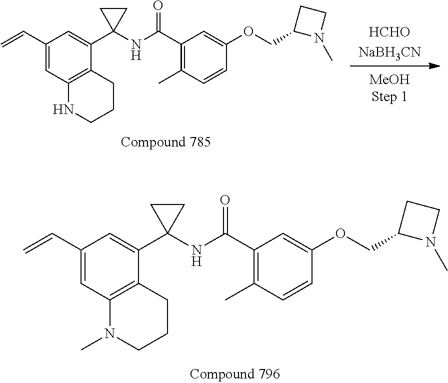

### Step 1: (S)-2-Methyl-N-(1-(1-methyl-7-vinyl-1,2,3,4-tetrahydroquinolin-5-yl)cyclo propyl)-5-((1-methylazetidin-2-yl)methoxy)benzamide (Compound 796)

To a solution of(S)-2-methyl-5-((1-methylazetidin-2-yl)methoxy)-N-(1-(7-vinyl-1,2,3,4-tetrahydroquinolin-5-yl)cyclopropyl)benzamide (20.0 mg, 46.3 μmol, 1.0 eq) in MeOH (2.0 mL) was added TEA (50.0 μL), followed by formaldehyde (3.76 mg, 46.3 μmol, 3.5 μL, 37% purity in water, 1.0 eq). The resulting mixture was adjusted to pH 6 with a small amount of AcOH. The mixture was stirred at 20° C. for 30 min, then NaBH₃CN (2.91 mg, 46.4 μmol, 1.0 eq) was added. The resulting reaction mixture was stirred at 20° C. for another 1 h. LCMS indicated that the starting material was completely consumed, and the desired mass was detected. The mixture was treated with H₂O (2.0 mL) and extracted with DCM (1.0 mL×5). The combined organic layers were dried over Na₂SO₄, filtered, and concentrated under vacuum to give a residue which was purified by preparative HPLC (Phenomenex Luna C18 column (100×40 mm, 5 μm); flow rate: 25 mL/min; gradient: 20%-50% B over 8 min; mobile phase A: 0.1% aqueous TFA, mobile phase B: acetonitrile). (S)-2-Methyl-N-(1-(1-methyl-7-vinyl-1,2,3,4-tetrahydroquinolin-5-yl)cyclopropyl)-5-((1-methyl azetidin-2-yl)methoxy)benzamide (3.00 mg, 5.36 μmol, 12% yield, TFA salt). M+H⁺=446.4 (LCMS); ¹H NMR (400 MHZ, DMSO-d₆) δ 10.05-9.84 (m, 1H), 8.72 (s, 1H), 7.16-7.10 (m, 1H), 7.01 (s, 1H), 6.97-6.90 (m, 1H), 6.79-6.74 (m, 1H), 6.67-6.55 (m, 2H), 5.70 (d, J=17.6 Hz, 1H), 5.14 (d, J=11.0 Hz, 1H), 4.70-4.57 (m, 1H), 4.33-4.22 (m, 2H), 4.09-4.00 (m, 1H), 3.94-3.83 (m, 1H), 3.18 (t, J=5.7 Hz, 2H), 2.95 (br t, J=6.4 Hz, 2H), 2.89-2.81 (m, 6H), 2.41-2.30 (m, 2H), 2.12 (s, 3H), 1.89 (quin, J=5.9 Hz, 2H), 1.12 (br s, 2H), 1.03 (br s, 2H).

### Example 405: (S)-2-Methyl-5-(2-(methylamino)propoxy)-N-(1-(7-(prop-1-en-2-yl)quinolin-5-yl)cyclopropyl)benzamide (Compound 660)

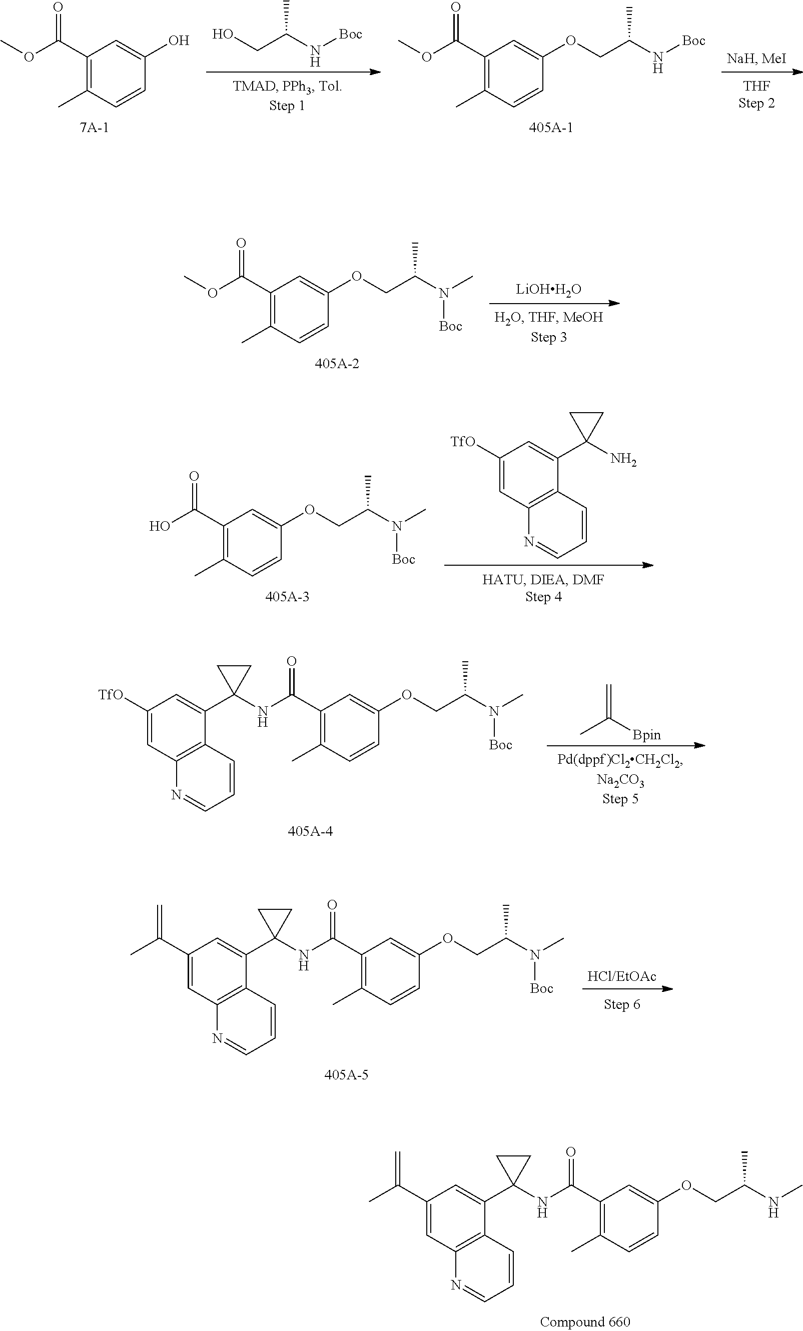

### Step 1: (S)-Methyl 5-(2-((tert-butoxycarbonyl)amino)propoxy)-2-methylbenzoate (405A-1)

To a solution of methyl 5-hydroxy-2-methylbenzoate (5.00 g, 30.1 mmol, 1.0 eq) and tert-butyl(S) (1-hydroxypropan-2-yl)carbamate (5.27 g, 30.1 mmol, 1.0 eq) in toluene (200 mL) were added TMAD (15.5 g, 90.3 mmol, 3.0 eq) and PPh₃ (23.7 g, 90.3 mmol, 3.0 eq). The mixture was stirred at 100° C. for 12 h under a N₂ atmosphere. LCMS indicated that the 31% of the starting material remained and 23% of the desired product was detected. The mixture was allowed to cool to room temperature, then concentrated under vacuum to give a residue which was treated with water (30 mL) and extracted with EtOAc (20 mL×3). The combined organic layers were dried over Na₂SO₄ filtered, and concentrated under vacuum to give a residue which was purified by flash silica gel chromatography using a gradient of EtOAc/petroleum ether from 0/1 to 1/19. (S)-Methyl 5-(2-((tert-butoxycarbonyl)amino)propoxy)-2-methylbenzoate (6.00 g, 18.5 mmol, 30% yield) was obtained as a white solid. M−100+H⁺=224.1 (LCMS).

### Step 2: (S)-Methyl 5-(2-((tert-butoxycarbonyl)(methyl)amino)propoxy)-2-methylbenzoate (405A-2)

To a solution of methyl(S)-5-(2-((tert-butoxycarbonyl)amino)propoxy)-2-methylbenzoate (3.00 g, 9.28 mmol, 1.0 eq) in THF (100 mL) were added NaH (556 mg, 13.9 mmol, 60% purity, 1.5 eq) and Mel (3.95 g, 27.8 mmol, 3.0 eq). The mixture was stirred at 20° C. for 12 h. LCMS indicated that the 27% of the starting material remained and 24% of the desired product was detected. The mixture was quenched with water (30 mL) and extracted with EtOAc (20 mL×3). The combined organic layers were dried over Na₂SO₄ filtered, and concentrated under vacuum to give a residue which was purified by flash silica gel chromatography using a gradient of EtOAc/petroleum ether from 0/1 to 1/4. (S)-Methyl 5-(2-((tert-butoxycarbonyl)(methyl)amino)propoxy)-2-methylbenzoate (3.00 g, 8.90 mmol, 95% yield) was obtained as a white solid. M−56+H⁺=282.2 (LCMS).

### Step 3: (S)-5-(2-((tert-Butoxycarbonyl)(methyl)amino)propoxy)-2-methylbenzoic acid (405A-3)

To a solution of methyl(S)-5-(2-((tert-butoxycarbonyl)(methyl)amino)propoxy)-2-methylbenzoate (3.00 g, 8.89 mmol, 1.0 eq) in THF (10 mL), H₂O (5.0 mL) and MeOH (7.5 mL) was added LiOH·H₂O (621 mg, 14.8 mmol, 3.0 eq). The mixture was stirred at 20° C. for 12 h. LCMS indicated that the starting material was completely consumed. THF and MeOH were concentrated under vacuum. The aqueous layer was adjusted to pH 5 with HCl (1 M aqueous), then extracted with EtOAc (20 mL×3). The combined organic layers were dried over Na₂SO₄, filtered, and concentrated under vacuum to give(S)-5-(2-((tert-butoxycarbonyl)(methyl)amino)propoxy)-2-methylbenzoic acid (1.67 g, 5.01 mmol, 99% yield) as a white solid. M−56+H⁺=268.2 (LCMS).

### Step 4: (S)-5-(1-(5-(2-((tert-Butoxycarbonyl)(methyl)amino)propoxy)-2-methylbenzamido)cyclopropyl)quinolin-7-yl trifluoromethanesulfonate (405A-4)

To a solution of 5-(1-aminocyclopropyl)quinolin-7-yl trifluoromethanesulfonate (300 mg, 902 μmol, 1.0 eq) and(S)-5-(2-((tert-butoxycarbonyl)(methyl)amino)propoxy)-2-methylbenzoic acid (291 mg, 902 μmol, 1.0 eq) in DMF (4.0 mL) were added HATU (514 mg, 1.35 mmol, 1.5 eq) and DIEA (350 mg, 2.71 mmol, 3.0 eq). The mixture was stirred at 20° C. for 12 h. LCMS indicated that the starting material was completely consumed. The mixture was treated with water (10 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/3. (S)-5-(1-(5-(2-((tert-Butoxycarbonyl)(methyl)amino)propoxy)-2-methylbenzamido)cyclopropyl)quinolin-7-yl trifluoromethanesulfonate (300 mg, 409 μmol, 45% yield) was obtained as a white solid. M+H⁺=638.3 (LCMS).

### Step 5: (S)-Methyl(1-(4-methyl-3-((1-(7-(prop-1-en-2-yl)quinolin-5-yl)cyclopropyl)carbamoyl)phenoxy)propan-2-yl)carbamate (405A-5)

To a solution of(S)-5-(1-(5-(2-((tert-butoxycarbonyl)(methyl)amino)propoxy)-2-methylbenzamido)cyclopropyl)quinolin-7-yl trifluoromethanesulfonate (130 mg, 203 μmol, 1.0 eq) and 4,4,5,5-tetramethyl-2-(prop-1-en-2-yl)-1,3,2-dioxaborolane (49.7 mg, 468 μmol, 2.9 eq) in a mixture of dioxane (1.0 mL) and H₂O (100 μL) were added Na₂CO₃ (8.00 mg, 76.2 μmol, 2.3 eq) and Pd(dppf)Cl₂·CH₂Cl₂ (16.6 mg, 20.3 μmol, 0.1 eq). The resulting mixture was stirred at 80° C. for 4 h under a N₂ atmosphere. LCMS indicated that the starting material was completely consumed, and the desired mass was detected. The mixture was allowed to cool to room temperature, then treated with water (10 mL) and extracted with EtOAc (10 mL×3). The combined organic layers were dried over Na₂SO₄, filtered, and concentrated under vacuum to give(S)-methyl(1-(4-methyl-3-((1-(7-(prop-1-en-2-yl)quinolin-5-yl)cyclopropyl)carbamoyl) phenoxy)propan-2-yl)carbamate (130 mg, crude) as a yellow oil. M+H⁺==530.4 (LCMS).

### Step 6: (S)-2-Methyl-5-(2-(methylamino)propoxy)-N-(1-(7-(prop-1-en-2-yl)quinolin-5-yl)cyclopropyl)benzamide (Compound 660)

To a solution of(S)-methyl(1-(4-methyl-3-((1-(7-(prop-1-en-2-yl)quinolin-5-yl)cyclopropyl)carbamoyl)phenoxy)propan-2-yl)carbamate (130 mg, 472 μmol, 1.0 eq) in EtOAc (500 μL) was added HCl/EtOAc (4 M, 1.5 mL). The resulting mixture was stirred at 20° C. for 1 h. LCMS indicated that the starting material was completely consumed. The mixture was concentrated under vacuum to give a residue which was purified by preparative HPLC (Phenomenex Gemini C18 column (80×30 mm, 3 μm); flow rate: 60 mL/min; gradient: 5%-30% B over 8 min; mobile phase A: 0.04% aqueous HCl, mobile phase B: acetonitrile). (S)-2-Methyl-5-(2-(methylamino)propoxy)-N-(1-(7-(prop-1-en-2-yl)quinolin-5-yl)cyclopropyl)benzamide (25.5 mg, 54.5 μmol, 22% yield) was obtained as a white solid. M+H⁺=430.3 (LCMS); ¹H NMR (400 MHZ, DMSO-d₆) δ 9.68-9.40 (m, 1H), 9.29 (s, 1H), 9.17 (br s, 1H), 9.14-8.75 (m, 2H), 8.26 (s, 1H), 8.18 (br s, 1H), 7.95 (br d, J=4.3 Hz, 1H), 7.09 (d, J=8.5 Hz, 1H), 6.91 (dd, J=2.7, 8.3 Hz, 1H), 6.72 (d, J=2.6 Hz, 1H), 5.83 (s, 1H), 5.48 (s, 1H), 4.13 (dd, J=4.0, 10.5 Hz, 1H), 4.06-4.01 (m, 1H), 3.44 (br s, 1H), 2.57-2.53 (m, 3H), 2.27 (s, 3H), 1.97 (s, 3H), 1.46-1.38 (m, 2H), 1.38-1.31 (m, 2H), 1.27 (d, J=6.7 Hz, 3H).

### Example 406: (S)—N-(1-(7-Acetylquinolin-5-yl)cyclopropyl)-2-methyl-5-((1-methylazetidin-2-yl)methoxy)benzamide (Compound 638)

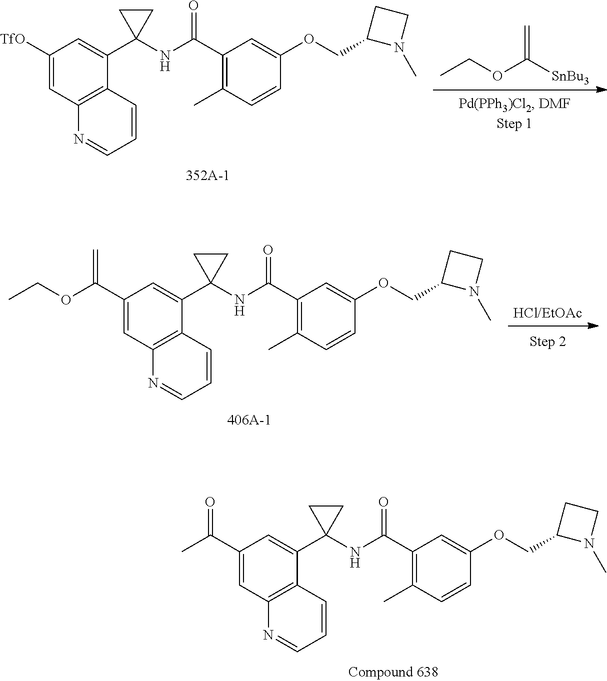

### Step 1: (S)—N-(1-(7-(1-Ethoxyvinyl)quinolin-5-yl)cyclopropyl)-2-methyl-5-((1-methylazetidin-2-yl)methoxy)benzamide (406A-1)

To a solution of(S)-5-(1-(2-methyl-5-((1-methylazetidin-2-yl)methoxy)benzamido)cyclopropyl)quinolin-7-yl trifluoromethanesulfonate (300 mg, 546 μmol, 1.0 eq) in DMF (6.0 mL) were added tributyl(1-ethoxyvinyl) stannane (394 mg, 1.09 mmol, 369 μL, 2.0 eq) and Pd(PPh₃)₂Cl₂ (38.3 mg, 54.6 μmol, 0.1 eq). The mixture was stirred at 60° C. for 15 h under a N₂ atmosphere. LCMS indicated that the starting material was completely consumed, and the desired mass was detected. The reaction mixture was allowed to cool to room temperature, poured into H₂O (15 mL), and extracted with EtOAc (20 mL×3). The combined organic layers were dried over Na₂SO₄, filtered, and concentrated under vacuum to give(S)—N-(1-(7-(1-ethoxyvinyl)quinolin-5-yl)cyclopropyl)-2-methyl-5-((1-methylazetidin-2-yl)methoxy) benzamide (300 mg) as a brown oil, which was used in the next step without further purification. M+H⁺=472.3 (LCMS).

### Step 2: (S)—N-(1-(7-Acetylquinolin-5-yl)cyclopropyl)-2-methyl-5-((1-methylazetidin-2-yl)methoxy)benzamide (Compound 638)

A mixture of(S)—N-(1-(7-(1-ethoxyvinyl)quinolin-5-yl)cyclopropyl)-2-methyl-5-((1-methylazetidin-2-yl)methoxy)benzamide (300 mg, 636 μmol, 1.0 eq) and HCl/EtOAc (4 M, 4.0 mL, 25 eq) was stirred at 20° C. for 1 h. LCMS indicated that the starting material was completely consumed, and the desired mass was detected. The mixture was concentrated under vacuum at 20° C. to give a residue which was purified by preparative HPLC (Phenomenex Luna column (80×30 mm, 3 μm); flow rate: 25 mL/min; gradient: 5%-25% B over 8 min; mobile phase A: 0.04% aqueous HCl, mobile phase B: acetonitrile). (S)—N-(1-(7-Acetylquinolin-5-yl)cyclopropyl)-2-methyl-5-((1-methylazetidin-2-yl)methoxy)benzamide (52.4 mg, 105 μmol, 17% yield, HCl salt) was obtained as a yellow solid. M+H⁺=444.1 (LCMS); ¹H NMR (400 MHz, DMSO-d₆) δ 9.31-9.21 (m, 2H), 9.11 (dd, J=1.4, 4.3 Hz, 1H), 8.62 (d, J=0.7 Hz, 1H), 8.35 (d, J=1.4 Hz, 1H), 7.83 (dd, J=4.3, 8.6 Hz, 1H), 7.13-7.06 (m, 1H), 6.95-6.87 (m, 1H), 6.75-6.68 (m, 1H), 4.66-4.54 (m, 1H), 4.30-4.18 (m, 2H), 4.01 (dt, J=5.1, 9.6 Hz, 1H), 3.85 (q, J=9.4 Hz, 1H), 2.82 (s, 3H), 2.76 (s, 3H), 2.39-2.28 (m, 2H), 1.93 (s, 3H), 1.42 (br s, 2H), 1.30-1.23 (m, 2H).

### Example 407: N-(1-(7-(1-Hydroxyethyl)quinolin-5-yl)cyclopropyl)-2-methyl-5-(((S)-1-methylazetidin-2-yl)methoxy)benzamide (Compound 676)

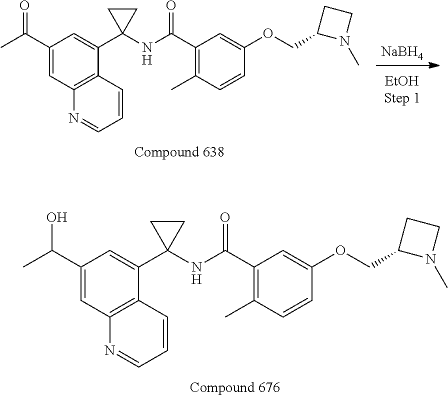

### Step 1: N-(1-(7-(1-Hydroxyethyl)quinolin-5-yl)cyclopropyl)-2-methyl-5-(((S)-1-methylazetidin-2-yl)methoxy)benzamide (Compound 676)

To a solution of(S)—N-(1-(7-acetylquinolin-5-yl)cyclopropyl)-2-methyl-5-((1-methylazetidin-2-yl)methoxy)benzamide (110 mg, 248 μmol, 1.0 eq) in EtOH (10 mL) was added NaBH₄ (18.8 mg, 496 μmol, 2.0 eq) at 0° C. Then the mixture was stirred at 20° C. for 5 h. LCMS indicated that the starting material was completely consumed, and the desired mass was detected. The reaction mixture was poured into with NH₄Cl aqueous (20 mL) and extracted with EtOAc (10 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 1%-20% B over 8 min; mobile phase A: 0.04% aqueous HCl, mobile phase B: acetonitrile). N-(1-(7-(1-Hydroxyethyl)quinolin-5-yl)cyclopropyl)-2-methyl-5-(((S)-1-methylazetidin-2-yl)methoxy)benzamide (27.0 mg, 55.5 μmol, 22% yield, HCl salt) was obtained as a white solid. M+H⁺=446.1 (LCMS); ¹H NMR (400 MHZ, CD₃OD) δ 10.13-9.93 (m, 1H), 9.34-9.18 (m, 1H), 8.45-8.32 (m, 1H), 8.29-8.22 (m, 1H), 8.21-8.14 (m, 1H), 7.19-7.09 (m, 1H), 7.04-6.95 (m, 1H), 6.92-6.82 (m, 1H), 5.18 (q, J=6.5 Hz, 1H), 4.77-4.68 (m, 1H), 4.38-4.15 (m, 3H), 3.98 (q, J=9.5 Hz, 1H), 2.96 (s, 3H), 2.62-2.51 (m, 2H), 2.03 (s, 3H), 1.67-1.55 (m, 5H), 1.48-1.43 (m, 2H).

### Example 408: (S)-5-(Azetidin-2-ylmethoxy)-N-(1-(7-ethynylquinolin-5-yl)cyclopropyl)-2-methylbenzamide (Compound 697)

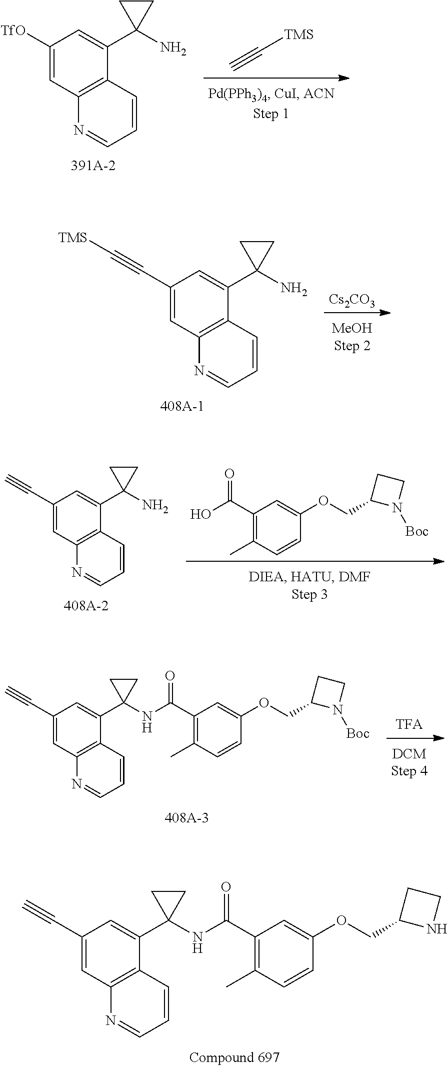

### Step 1: 1-(7-((Trimethylsilyl) ethynyl)quinolin-5-yl)cyclopropan-1-amine (408A-1)

To a solution of 5-(1-aminocyclopropyl)quinolin-7-yl trifluoromethanesulfonate (200 mg, 602 μmol, 1.0 eq) in acetonitrile (10 mL) were added TEA (82.7 mg, 1.81 mmol, 251 μL, 3.0 eq), ethynyltrimethylsilane (118 mg, 1.20 mmol, 167 μL, 2.0 eq), CuI (11.5 mg, 60.2 μmol, 0.1 eq) and Pd(PPh₃)₄ (69.6 mg, 60.2 μmol, 0.1 eq). The mixture was stirred at 80° C. for 16 h under a N₂ atmosphere. LCMS indicated that the starting material was completely consumed, and the desired mass was detected. The mixture was treated with water (10 mL) and extracted with EtOAc (10 mL×4). The combined organic layers were dried over Na₂SO₄, filtered, and concentrated under vacuum to give a residue which was purified by flash silica gel chromatography using a gradient of EtOAc/petroleum ether from 0/1 to 1/3. 1-(7-((Trimethylsilyl) ethynyl)quinolin-5-yl)cyclopropan-1-amine (115 mg, 410 μmol, 68% yield) was obtained as a white solid.

### Step 2: 1-(7-Ethynylquinolin-5-yl)cyclopropan-1-amine (408A-2)

To a solution of 1-(7-((trimethylsilyl) ethynyl)quinolin-5-yl)cyclopropan-1-amine (100 mg, 357 μmol, 1.0 eq) in MeOH (2.0 mL) was added Cs₂CO₃ (232 mg, 713 μmol, 2.0 eq). The mixture was stirred at 25° C. for 1 h. LCMS indicated that the starting material was completely consumed, and the desired mass was detected. The reaction mixture was poured into H₂O (1.0 mL) and extracted with DCM (1.0 mL×4). The combined organic layers were dried over Na₂SO₄, filtered, and concentrated under vacuum to give the crude 1-(7-ethynylquinolin-5-yl)cyclopropan-1-amine (100 mg) as a white solid.

### Step 3: tert-Butyl(S)-2-((3-((1-(7-ethynylquinolin-5-yl)cyclopropyl)carbamoyl)-4-methylphenoxy)methyl)azetidine-1-carboxylate (408A-3)

To a solution of 1-(7-ethynylquinolin-5-yl)cyclopropan-1-amine (100 mg, 480 μmol, 1.0 eq) and(S)-5-((1-(tert-butoxycarbonyl)azetidin-2-yl)methoxy)-2-methylbenzoic acid (154 mg, 480 μmol, 1.0 eq) in DMF (2.0 mL) were added DIEA (186 mg, 1.44 mmol, 251 μL, 3.0 eq) and HATU (456 mg, 1.20 mmol, 2.5 eq). The resulting mixture was stirred at 25° C. for 1 h. LCMS indicated that the starting material was completely consumed, and the desired mass was detected. The mixture was treated with H₂O (10 mL) and extracted with EtOAc (2.0 mL×4). The combined organic layers were dried over Na₂SO₄, filtered, and concentrated under vacuum to give a residue which was purified by preparative TLC (EtOAc/petroleum ether=1/1, R_{f}=0.4). tert-Butyl(S)-2-((3-((1-(7-ethynylquinolin-5-yl)cyclopropyl)carbamoyl)-4-methylphenoxy)methyl)azetidine-1-carboxylate (90.0 mg, 176 μmol, 37% yield) was obtained as a white solid.

### Step 4: (S)-5-(Azetidin-2-ylmethoxy)-N-(1-(7-ethynylquinolin-5-yl)cyclopropyl)-2-methylbenzamide (Compound 697)

To a solution of tert-butyl(S)-2-((3-((1-(7-ethynylquinolin-5-yl)cyclopropyl)carbamoyl)-4-methylphenoxy)methyl)azetidine-1-carboxylate (90.0 mg, 176 μmol, 1.0 eq) in DCM (2.0 mL) was added TFA (361 mg, 3.17 mmol, 234 μL, 18 eq). The mixture was stirred at 25° C. for 1 h. LCMS indicated that the starting material was completely consumed, and the desired mass was detected. The mixture was concentrated under vacuum at 25° C. to give a residue which was purified by preparative HPLC (Phenomenex C18 column (75×30 mm, 3 μm); flow rate: 25 mL/min; gradient: 1%-30% B over 8 min; mobile phase A: 0.1% aqueous TFA, mobile phase B: acetonitrile). (S)-5-(Azetidin-2-ylmethoxy)-N-(1-(7-ethynylquinolin-5-yl)cyclopropyl)-2-methylbenzamide (21.8 mg, 41.5 μmol, 24% yield, TFA salt) was obtained as a yellow solid. M+H⁺=412.1 (LCMS); ¹H NMR (400 MHz, DMSO-d₆) δ 9.16 (s, 1H), 9.07 (d, J=8.8 Hz, 1H), 8.97 (dd, J=1.5, 4.1 Hz, 1H), 8.89-8.70 (m, 2H), 8.06 (s, 1H), 7.87 (d, J=1.6 Hz, 1H), 7.65 (dd, J=4.2, 8.6 Hz, 1H), 7.10 (d, J=8.5 Hz, 1H), 6.91 (dd, J=2.8, 8.4 Hz, 1H), 6.70 (d, J=2.6 Hz, 1H), 4.72-4.57 (m, 1H), 4.46 (s, 1H), 4.29-4.19 (m, 1H), 4.17-4.09 (m, 1H), 4.02-3.88 (m, 1H), 3.87-3.79 (m, 1H), 2.47-2.41 (m, 1H), 2.38-2.29 (m, 1H), 1.95 (s, 3H), 1.36 (br s, 2H), 1.30-1.21 (m, 2H).

### Example 409: (S)—N-(1-(7-Ethynylquinolin-5-yl)cyclopropyl)-2-methyl-5-((1-methylazetidin-2-yl)methoxy)benzamide (Compound 620)

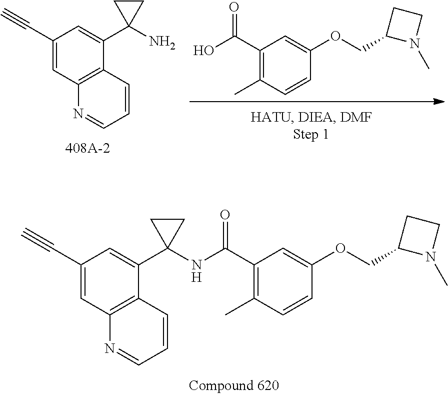 [SMILES: C#Cc1cc(C2(N)CC2)c2cccnc2c1 ; Cc1ccc(OC[C@@H]2CCN2C)cc1C(=O)O ; C#Cc1cc(C2(NC(=O)c3cc(OC[C@@H]4CCN4C)ccc3C)CC2)c2cccnc2c1]

### Step 1: (S)—N-(1-(7-Ethynylquinolin-5-yl)cyclopropyl)-2-methyl-5-((1-methylazetidin-2-yl)methoxy)benzamide (Compound 620)

To a solution of(S)-2-methyl-5-((1-methylazetidin-2-yl)methoxy)benzoic acid (192 mg, 816 μmol, 1.0 eq) and 1-(7-ethynyl-5-quinolyl)cyclopropanamine (170 mg, 816 μmol, 1.0 eq) in DMF (3.0 mL) were added DIEA (316 mg, 2.45 mmol, 427 μL, 3.0 eq) and HATU (776 mg, 2.04 mmol, 2.5 eq). The mixture was stirred at 20° C. for 12 h. LCMS indicated that the starting material was completely consumed, and the desired mass was detected. The mixture was filtered. The filtrate obtained was purified by preparative HPLC (Xbridge BEH C18 (100× 30 mm, 10 μm); flow rate: 60 mL/min; gradient: 30%-50% B over 8 min; mobile phase A: 10 mM aqueous NH₄HCO₃, mobile phase B: acetonitrile). The residue obtained was diluted with a mixture of acetonitrile (1.0 mL), H₂O (4.0 mL), and HCl (1 M aqueous, 133 μL). The resulting mixture was lyophilized to give(S)—N-(1-(7-ethynylquinolin-5-yl)cyclopropyl)-2-methyl-5-((1-methylazetidin-2-yl)methoxy)benzamide (51.4 mg, 103 μmol, 13% yield, HCl salt) as a white solid. M+H⁺=426.1 (LCMS); ¹H NMR (400 MHZ, DMSO-d₆) δ 10.78-10.35 (m, 1H), 9.21 (s, 1H), 9.07 (d, J=8.5 Hz, 1H), 8.96 (dd, J=1.3, 4.1 Hz, 1H), 8.05 (s, 1H), 7.86 (d, J=1.5 Hz, 1H), 7.63 (dd, J=4.2, 8.6 Hz, 1H), 7.09 (d, J=8.5 Hz, 1H), 6.91 (br d, J=7.1 Hz, 1H), 6.72 (br s, 1H), 4.60 (br d, J=7.4 Hz, 1H), 4.46 (s, 1H), 4.37-4.28 (m, 1H), 4.26-4.17 (m, 1H), 4.04-3.95 (m, 1H), 3.92-3.79 (m, 1H), 2.81 (br s, 3H), 2.40-2.26 (m, 2H), 1.94 (s, 3H), 1.37 (br s, 2H), 1.25 (br s, 2H).

### Example 410: 5-(Azetidin-3-ylamino)-2-methyl-N-(1-(5,6,7,8-tetrahydronaphthalen-1-yl)ethyl)benzamide (Compound 696)

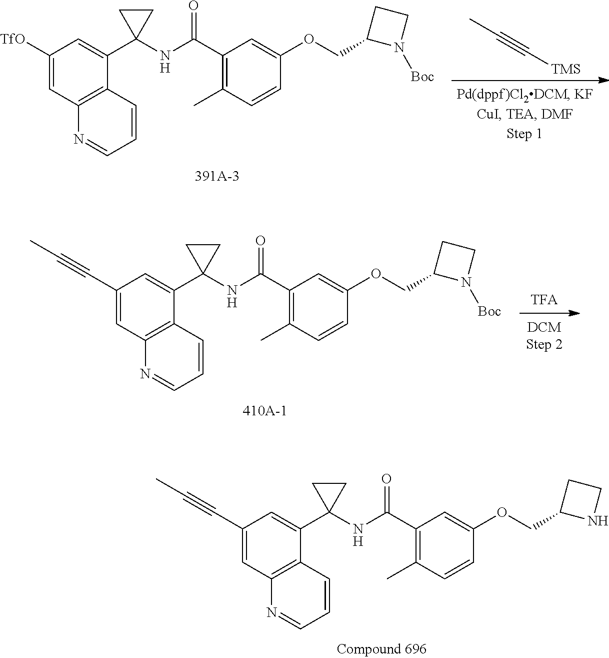

### Step 1: tert-Butyl(S)-2-((4-methyl-3-((1-(7-(prop-1-yn-1-yl)quinolin-5-yl)cyclopropyl)carbamoyl)phenoxy)methyl)azetidine-1-carboxylate (410A-1)

To a mixture of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (100 mg, 157 μmol, 1.0 eq) and 1-methyl-3-(4,4,5,5-tetramethyl-1,3,2-dioxaborolan-2-yl)-1H-pyrazole (68.7 mg, 330 μmol, 2.1 eq) in a mixture of H₂O (1.0 mL) and dioxane (6.0 mL) were added TEA (6.37 mg, 62.9 μmol, 7.0 μL, 0.3 eq), KF (27.4 mg, 472 μmol, 3.0 eq), Pd(dppf)Cl₂·CH₂Cl₂ (38.5 mg, 47.2 μmol, 0.3 eq) and CuI (4.49 mg, 23.6 μmol, 0.15 eq). The mixture was degassed and purged with N₂ three times. The resulting mixture was stirred at 80° C. for 14 h under a N₂ atmosphere. LCMS indicated that the starting material was completely consumed, and the desired mass was detected. The reaction mixture was allowed to cool to room temperature, poured into H₂O (10 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/2. tert-Butyl(S)-2-((3-((1-(7-((tert-butoxycarbonyl)amino)quinolin-5-yl)cyclopropyl)carbamoyl)-4-methylphenoxy)methyl)azetidine-1-carboxylate (80.0 mg, 152 μmol, 48% yield) was obtained as a yellow solid. M+H⁺=526.3 (LCMS); ¹H NMR (400 MHZ, DMSO-d₆) δ 9.13 (s, 1H), 9.02 (d, J=8.0 Hz, 1H), 8.91 (dd, J=1.6, 4.2 Hz, 1H), 7.91 (s, 1H), 7.80 (d, J=1.6 Hz, 1H), 7.58 (dd, J=4.2, 8.6 Hz, 1H), 7.04 (d, J=8.5 Hz, 1H), 6.88 (dd, J=2.8, 8.4 Hz, 1H), 6.66 (d, J=2.6 Hz, 1H), 4.42-4.34 (m, 1H), 4.14 (dd, J=4.8, 10.3 Hz, 1H), 4.00-3.96 (m, 1H), 3.77-3.69 (m, 2H), 2.33-2.25 (m, 1H), 2.13 (s, 3H), 2.10-2.03 (m, 1H), 1.93 (s, 3H), 1.39-1.25 (m, 13H).

### Step 2: (S)-5-(Azetidin-2-ylmethoxy)-2-methyl-N-(1-(7-(prop-1-yn-1-yl)quinolin-5-yl)cyclopropyl)benzamide (Compound 696)

To a solution of tert-butyl(S)-2-((4-methyl-3-((1-(7-(1-methyl-1H-pyrazol-3-yl)quinolin-5-yl)cyclopropyl)carbamoyl)phenoxy)methyl)azetidine-1-carboxylate (80.0 mg, 152 μmol, 1.0 eq) in DCM (1.0 mL) was added TFA (800 μL). The mixture was stirred at 20° C. for 1 h. LCMS indicated that the starting material was completely consumed, and the desired mass was detected. The mixture was concentrated under vacuum at 20° C. to give a residue which was purified by preparative HPLC (Phenomenex Luna C18 column (75×30 mm, 3 μm); flow rate: 25 mL/min; gradient: 1%-35% B over 8 min; mobile phase A: 0.1% aqueous TFA, mobile phase B: acetonitrile). (S)-5-(Azetidin-2-ylmethoxy)-2-methyl-N-(1-(7-(prop-1-yn-1-yl)quinolin-5-yl)cyclopropyl)benzamide (19.9 mg, 46.7 μmol, 31% yield, TFA salt) was obtained as a white solid. M+H⁺=426.1 (LCMS); ¹H NMR (400 MHZ, DMSO-d₆) δ 9.15 (s, 1H), 9.05 (d, J=8.4 Hz, 1H), 8.94 (dd, J=1.5, 4.1 Hz, 1H), 8.90-8.74 (m, 2H), 7.93 (s, 1H), 7.82 (d, J=1.6 Hz, 1H), 7.61 (dd, J=4.2, 8.6 Hz, 1H), 7.09 (d, J=8.5 Hz, 1H), 6.91 (dd, J=2.6, 8.4 Hz, 1H), 6.70 (d, J=2.6 Hz, 1H), 4.69-4.60 (m, 1H), 4.26-4.20 (m, 1H), 4.16-4.11 (m, 1H), 3.93 (br s, 2H), 2.46-2.30 (m, 2H), 2.14 (s, 3H), 1.95 (s, 3H), 1.35 (br s, 2H), 1.23 (br s, 2H).

### Example 411: (S)-2-Methyl-5-((1-methylazetidin-2-yl)methoxy)-N-(1-(7-(3,3,3-trifluoroprop-1-yn-1-yl)quinolin-5-yl)cyclopropyl)benzamide (Compound 803)

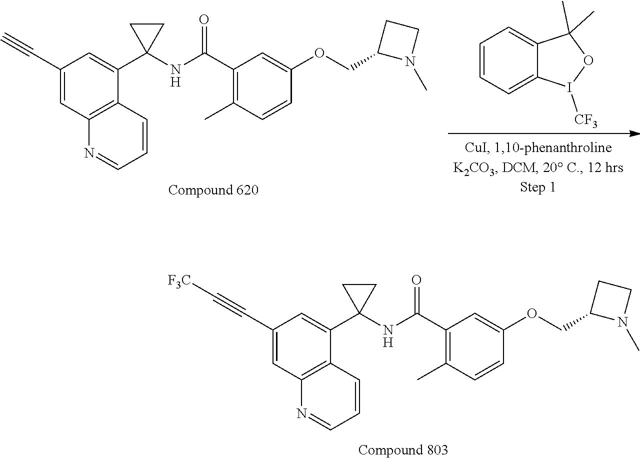 [SMILES: C#Cc1cc(C2(NC(=O)c3cc(OC[C@@H]4CCN4C)ccc3C)CC2)c2cccnc2c1 ; CC1(C)OI(C(F)(F)F)c2ccccc21 ; Cc1ccc(OC[C@@H]2CCN2C)cc1C(=O)NC1(c2cc(C#CC(F)(F)F)cc3ncccc23)CC1]

### Step 1: N-(1-(7-(1-Hydroxyethyl)quinolin-5-yl)cyclopropyl)-2-methyl-5-(((S)-1-methylazetidin-2-yl)methoxy)benzamide (Compound 803)

To a solution of CuI (3.58 mg, 18.8 μmol, 0.2 eq), 1,10-phenanthroline (6.78 mg, 37.6 μmol, 0.4 eq), K₂CO₃ (26.0 mg, 188 μmol, 2.0 eq) and 3,3-dimethyl-1-(trifluoromethyl)-1,3-dihydro-113-benzo[d][1,2]iodaoxole (31.0 mg, 94.0 μmol, 1.0 eq) in DCM (3.0 mL) was added dropwise a solution of(S)—N-(1-(7-ethynylquinolin-5-yl)cyclopropyl)-2-methyl-5-((1-methyl azetidin-2-yl)methoxy)benzamide (40.0 mg, 94.0 μmol, 1.0 eq) in DCM (1.0 mL). The mixture was stirred at 20° C. for 15 h. LCMS indicated that the starting material was completely consumed, and the desired mass was detected. The reaction mixture was poured into H₂O (10 mL) and extracted with DCM (5.0 mL×3). The combined organic layers were dried over Na₂SO₄, filtered, and concentrated under vacuum to give a residue which was purified by preparative HPLC (Phenomenex Luna column (80×30 mm, 3 μm); flow rate: 25 mL/min; gradient: 25%-55% B over 8 min; mobile phase A: 0.04% aqueous HCl, mobile phase B: acetonitrile). N-(1-(7-(1-Hydroxyethyl)quinolin-5-yl)cyclopropyl)-2-methyl-5-(((S)-1-methylazetidin-2-yl)methoxy)benzamide (18.1 mg, 33.8 μmol, 36% yield, HCl salt) was obtained as a white solid. M+H⁺=494.2 (LCMS); ¹H NMR (400 MHZ, DMSO-d₆) δ 10.76-10.43 (m, 1H), 9.27 (s, 1H), 9.15 (d, J=8.8 Hz, 1H), 9.06 (d, J=4.1 Hz, 1H), 8.39 (s, 1H), 8.03 (s, 1H), 7.76 (dd, J=4.2, 8.6 Hz, 1H), 7.13-7.06 (m, 1H), 6.97-6.89 (m, 1H), 6.78-6.71 (m, 1H), 4.68-4.56 (m, 1H), 4.33 (dd, J=7.8, 11.3 Hz, 1H), 4.25-4.17 (m, 1H), 4.06-3.94 (m, 1H), 3.90-3.79 (m, 1H), 2.81 (d, J=5.0 Hz, 3H), 2.40-2.28 (m, 2H), 1.93 (s, 3H), 1.43-1.35 (m, 2H), 1.30 (br s, 2H).

### Example 412: (S)—N-(1-(7-cyanoquinolin-5-yl)cyclopropyl)-2-methyl-5-((1-methyl azetidin-2-yl)methoxy)benzamide (Compound 738)

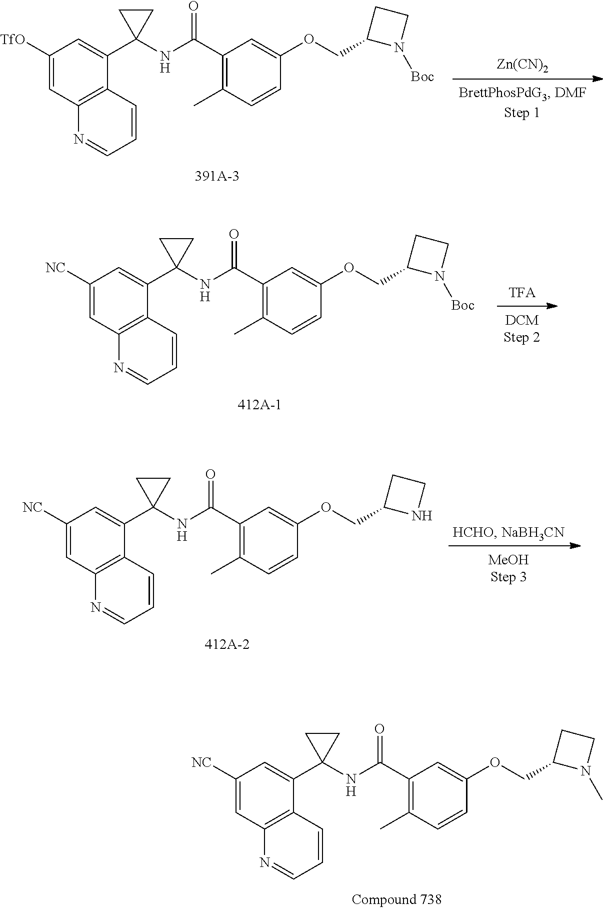

### Step 1: tert-Butyl(S)-2-((3-((1-(7-cyanoquinolin-5-yl)cyclopropyl)carbamoyl)-4-methylphenoxy)methyl)azetidine-1-carboxylate (412A-1)

To a solution of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (200 mg, 315 μmol, 1.0 eq) in DMF (10 mL) were added Zn(CN)₂ (111 mg, 944 μmol, 3.0 eq), BrettPhosPdG₃ (28.5 mg, 31.5 μmol, 0.1) and BrettPhos (33.8 mg, 62.9 μmol, 0.2 eq). The mixture was stirred at 80° C. for 12 h under a N₂ atmosphere. LCMS indicated that the starting material was completely consumed, and the desired mass was detected. The reaction mixture was poured into H₂O (20 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. tert-Butyl(S)-2-((3-((1-(7-cyanoquinolin-5-yl)cyclopropyl)carbamoyl)-4-methylphenoxy)methyl)azetidine-1-carboxylate (80.0 mg, 156 μmol, 49% yield) was obtained as a white solid. M+H⁺=513.2 (LCMS).

### Step 2: (S)-5-(Azetidin-2-ylmethoxy)-N-(1-(7-cyanoquinolin-5-yl)cyclopropyl)-2-methylbenzamide (412A-2)

To a solution of tert-butyl(S)-2-((3-((1-(7-cyanoquinolin-5-yl)cyclopropyl)carbamoyl)-4-methylphenoxy)methyl)azetidine-1-carboxylate (40.0 mg, 78.0 μmol, 1.0 eq) in DCM (2.0 mL) was added TFA (0.2 mL). The mixture was stirred at 25° C. for 1 h. LCMS indicated that the starting material was completely consumed, and the desired mass was detected. The mixture was concentrated under vacuum at 20° C. to give the crude(S)-5-(azetidin-2-ylmethoxy)-N-(1-(7-cyanoquinolin-5-yl)cyclopropyl)-2-methylbenzamide (30.0 mg, TFA salt) as a white solid. M+H⁺=413.2 (LCMS).

### Step 3: (S)—N-(1-(7-Cyanoquinolin-5-yl)cyclopropyl)-2-methyl-5-((1-methylazetidin-2-yl)methoxy)benzamide (Compound 738)

To a solution of(S)-5-(azetidin-2-ylmethoxy)-N-(1-(7-cyanoquinolin-5-yl)cyclopropyl)-2-methylbenzamide (30.0 mg, 56.9 μmol, 1.0 eq, TFA salt) in MeOH (4.0 mL) was added TEA (5.77 mg, 56.9 μmol, 7.93 μL, 1.0 eq), followed by formaldehyde (3.42 mg, 113.9 μmol, 3.14 μL, 2.0 eq, 37% aqueous). The resulting mixture was adjusted to pH 6 with a small amount of AcOH (3.42 mg, 56.9 μmol, 3.26 μL, 1 eq). The mixture was stirred at 25° C. for 30 min, then NaBH₃CN (17.9 mg, 284.90 μmol, 5.0 eq) was added. The reaction mixture was stirred at 25° C. for another 2 h. LCMS indicated that the starting material was completely consumed, and the desired mass was detected. The reaction mixture was poured into H₂O (5 mL) and extracted with DCM (2.0 mL×3). The combined organic layers were dried over Na₂SO₄, filtered, and concentrated under vacuum to give a residue which was purified by preparative HPLC (Phenomenex Luna C18 column (80×30 mm, 3 μm); flow rate: 25 mL/min; gradient: 5%-30% B over 8 min; mobile phase A: 0.1% aqueous HCl, mobile phase B: acetonitrile). (S)—N-(1-(7-Cyanoquinolin-5-yl)cyclopropyl)-2-methyl-5-((1-methylazetidin-2-yl)methoxy)benzamide (18.5 mg, 39.9 μmol, 70% yield, HCl salt) was obtained as a white solid. M+H⁺=427.1 (LCMS); ¹H NMR (400 MHZ, DMSO-d₆) δ 10.81-10.53 (m, 1H), 9.30 (s, 1H), 9.18 (d, J=8.5 Hz, 1H), 9.09 (dd, J=1.4, 4.1 Hz, 1H), 8.54 (s, 1H), 8.09 (d, J=1.6 Hz, 1H), 7.81 (dd, J=4.1, 8.6 Hz, 1H), 7.08 (d, J=8.4 Hz, 1H), 6.98-6.88 (m, 1H), 6.73 (d, J=2.6 Hz, 1H), 4.61 (br s, 1H), 4.42-4.14 (m, 2H), 4.04-3.79 (m, 2H), 2.85-2.64 (m, 3H), 2.41-2.24 (m, 2H), 1.93 (s, 3H), 1.43-1.28 (m, 4H).

### Example 413: (S)-5-(Azetidin-2-ylmethoxy)-N-(1-(7-ethylquinolin-5-yl)cyclopropyl)-2-methylbenzamide (Compound 677)

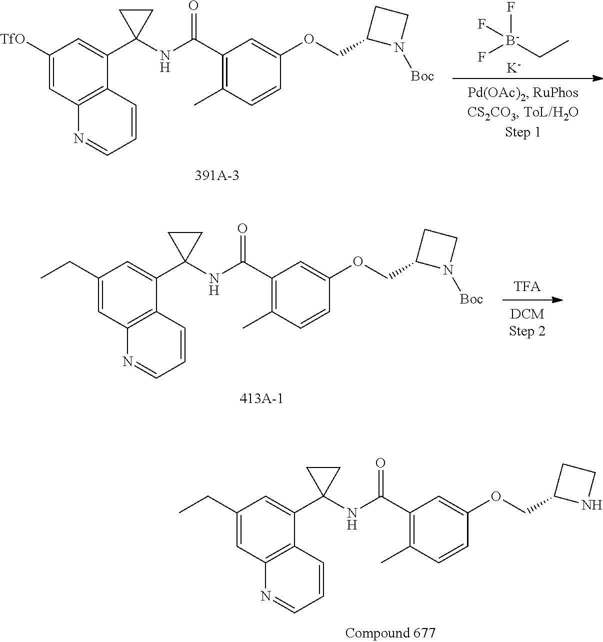

### Step 1: tert-Butyl(S)-2-((3-((1-(7-ethylquinolin-5-yl)cyclopropyl)carbamoyl)-4-methylphenoxy)methyl)azetidine-1-carboxylate (413A-1)

A solution of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (50.0 mg, 78.7 μmol, 1.0 eq), potassium ethyltrifluoroborate (11.8 mg, 86.5 μmol, 1.1 eq), Pd(OAc) 2 (1.77 mg, 7.87 μmol, 0.1 eq), RuPhos (7.34 mg, 15.7 μmol, 0.2 eq) and CS₂CO₃ (76.9 mg, 236 μmol, 3.0 eq) in a mixture of toluene (3.0 mL) and H₂O (0.3 mL) was degassed and purged with N₂ three times. The resulting mixture was stirred at 110° C. for 12 h under a N₂ atmosphere. LCMS indicated that the starting material was completely consumed, and the desired mass was detected. The mixture was allowed to cool to room temperature, poured into H₂O (5.0 mL) and extracted with EtOAc (4.0 mL×3). The combined organic layers were dried over Na₂SO₄, filtered, and concentrated under vacuum to give a residue which was purified by preparative TLC (EtOAc/petroleum ether=1/1, R_{f}=0.5). tert-Buty(S)-2-((3-((1-(7-ethylquinolin-5-yl)cyclopropyl)carbamoyl)-4-methylphenoxy)methyl)azetidine-1-carboxylate (63.0 mg, 122 μmol, 78% yield) was obtained as a white solid. M+H⁺=516.3 (LCMS).

### Step 2: (S)-5-(Azetidin-2-ylmethoxy)-N-(1-(7-ethylquinolin-5-yl)cyclopropyl)-2-methyl benzamide (Compound 677)

To a solution of tert-butyl(S)-2-((3-((1-(7-ethylquinolin-5-yl)cyclopropyl)carbamoyl)-4-methylphenoxy)methyl)azetidine-1-carboxylate (63.0 mg, 122.18 μmol, 1.0 eq) in DCM (3.0 mL) was added TFA (3.08 g, 27.0 mmol, 2.0 mL). The mixture was stirred at 20° C. for 1 h. LCMS indicated that the starting material was completely consumed. The combined organic layers were dried over Na₂SO₄, filtered, and concentrated under vacuum to give a residue which was purified by preparative HPLC (Phenomenex Gemini C18 (75×30 mm, 3 μm); flow rate: 25 mL/min; gradient: 15%-45% B over 8 min; mobile phase A: 0.1% aqueous TFA, mobile phase B acetonitrile). (S)-5-(Azetidin-2-ylmethoxy)-N-(1-(7-ethylquinolin-5-yl)cyclopropyl)-2-methylbenzamide (30.0 mg, 56.7 μmol, 47% yield, TFA salt) was obtained as a white solid. M+H⁺=416.3 (LCMS); ¹H NMR (400 MHZ, DMSO-d₆) δ 9.45 (br d, J=8.6 Hz, 1H), 9.19 (s, 1H), 9.08 (dd, J=1.3, 4.8 Hz, 1H), 7.95 (s, 1H), 7.90-7.82 (m, 2H), 7.09 (d, J=8.6 Hz, 1H), 6.91 (dd, J=2.7, 8.3 Hz, 1H), 6.70 (d, J=2.7 Hz, 1H), 4.69-4.58 (m, 1H), 4.28-4.17 (m, 1H), 4.16-4.08 (m, 1H), 3.98-3.77 (m, 2H), 2.89 (q, J=7.5 Hz, 2H), 2.48-2.42 (m, 1H), 2.40-2.28 (m, 1H), 1.95 (s, 3H), 1.40 (br s, 2H), 1.34-1.23 (m, 5H).

### Example 414: (S)-5-(Azetidin-2-ylmethoxy)-N-(1-(7-ethyl-1,2,3,4-tetrahydroquinolin-5-yl)cyclopropyl)-2-methylbenzamide (Compound 692)

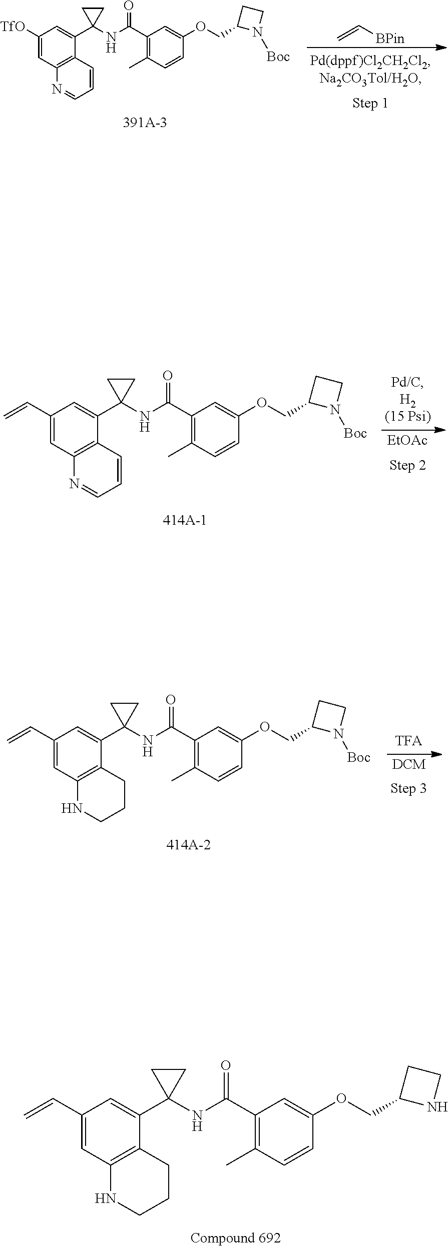

### Step 1: tert-Butyl(S)-2-((4-methyl-3-((1-(7-vinylquinolin-5-yl)cyclopropyl)carbamoyl)phenoxy)methyl)azetidine-1-carboxylate (414A-1)

A mixture of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (150 mg, 236 μmol, 1.0 eq), 4,4,5,5-tetramethyl-2-vinyl-1,3,2-dioxaborolane (72.7 mg, 472 μmol, 80.1 μL, 2.0 eq), Pd(dppf)Cl₂·CH₂Cl₂ (19.3 mg, 23.6 μmol, 0.1 eq), and Na₂CO₃ (57.5 mg, 543 μmol, 2.3 eq) in a mixture of dioxane (6.0 mL) and H₂O (1.0 mL) was degassed and purged with N₂ three times. The resulting mixture was stirred at 80° C. for 16 h under a N₂ atmosphere. LCMS indicated that the starting material was completely consumed. The mixture was poured into H₂O (10 mL) and extracted with EtOAc (5.0 mL×2). The combined organic layers were dried over Na₂SO₄, filtered, and concentrated under vacuum to give a residue which was purified by flash silica gel chromatography using a gradient of EtOAc/petroleum ether from 0/1 to 1/1. tert-Butyl(S)-2-((4-methyl-3-((1-(7-(((trifluoromethyl)sulfonyl)oxy)quinolin-5-yl)cyclopropyl)carbamoyl) phenoxy)methyl)azetidine-1-carboxylate (80.0 mg, 156 μmol, 66% yield) was obtained as a yellow oil. M+H⁺=514.3 (LCMS).

### Step 2: tert-Butyl(S)-2-((3-((1-(7-ethyl-1,2,3,4-tetrahydroquinolin-5-yl)cyclopropyl) carbamoyl)-4-methylphenoxy)methyl)azetidine-1-carboxylate (414A-2)

To a solution of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (80.0 mg, 156 μmol, 1.0 eq) in EtOAc (8.0 mL) was added 10% palladium on carbon (50.0 mg). The mixture was degassed and purged with H₂ three times and stirred at 20° C. for 2 h under a H₂ (15 psi) atmosphere. LCMS indicated that the starting material was completely consumed, and the desired mass was detected. The suspension was filtered through a pad of Celite, and the filter cake was washed with EtOAc (10 mL×3). The combined organic layers were concentrated under vacuum to give the crude product tert-butyl(S)-2-((3-((1-(7-ethyl-1,2,3,4-tetrahydro quinolin-5-yl)cyclopropyl)carbamoyl)-4-methylphenoxy)methyl)azetidine-1-carboxylate (30.0 mg, 57.7 μmol, 37% yield) as a white solid. M+H⁺=520.4 (LCMS); ¹H NMR (400 MHz, DMSO-d₆) δ 8.74 (s, 1H), 7.08 (d, J=8.5 Hz, 2H), 6.90 (dd, J=2.6, 8.3 Hz, 1H), 6.74 (d, J=2.6 Hz, 1H), 6.68-6.39 (m, 1H), 4.46-4.38 (m, 1H), 4.18 (dd, J=4.8, 10.4 Hz, 1H), 4.03 (dd, J=2.8, 10.4 Hz, 1H), 3.81-3.67 (m, 3H), 3.30-3.23 (m, 3H), 3.03-2.86 (m, 3H), 2.32-2.23 (m, 1H), 2.10 (s, 3H), 1.94-1.86 (m, 2H), 1.34 (s, 9H), 1.17-1.11 (m, 5H), 1.03 (br s, 2H).

### Step 3: (S)-5-(Azetidin-2-ylmethoxy)-N-(1-(7-ethyl-1,2,3,4-tetrahydroquinolin-5-yl)cyclopropyl)-2-methylbenzamide (Compound 692)

To a solution of tert-butyl(S)-2-((3-((1-(7-ethyl-1,2,3,4-tetrahydroquinolin-5-yl)cyclopropyl) carbamoyl)-4-methylphenoxy)methyl)azetidine-1-carboxylate (20.0 mg, 38.5 μmol, 1.0 eq) in DCM (4.0 mL) was added TFA (1.0 mL). The mixture was stirred at 20° C. for 1 h. LCMS indicated that the starting material was completely consumed, and the desired mass was detected. The reaction mixture was concentrated under vacuum at 20° C. to give a residue which was purified by preparative HPLC (Phenomenex Gemini C18 (75×30 mm, 3 μm); flow rate: 25 mL/min; gradient: 1%-30% B over 8 min; mobile phase A: 0.1% aqueous TFA, mobile phase B: acetonitrile). (S)-5-(Azetidin-2-ylmethoxy)-N-(1-(7-ethyl-1,2,3,4-tetrahydroquinolin-5-yl)cyclopropyl)-2-methylbenzamide (4.80 mg, 11.4 μmol, 30% yield) was obtained as a white solid. M+H⁺=420.4 (LCMS); ¹H NMR (400 MHZ, DMSO-d₆) δ 8.94-8.76 (m, 2H), 8.69 (s, 1H), 7.13 (d, J=8.4 Hz, 1H), 6.93 (dd, J=2.6, 8.4 Hz, 1H), 6.87-6.71 (m, 2H), 4.73-4.64 (m, 1H), 4.32-4.25 (m, 1H), 4.22-4.15 (m, 1H), 3.96-3.85 (m, 2H), 3.20 (br s, 2H), 2.92 (br t, J=5.9 Hz, 2H), 2.46-2.31 (m, 4H), 2.13 (s, 3H), 1.91-1.81 (m, 2H), 1.16-1.08 (m, 5H), 1.03-0.99 (m, 2H).

### Example 415: (S)—N-(1-(6-Methoxyquinolin-8-yl)cyclopropyl)-2-methyl-5-((1-methylazetidin-2-yl)methoxy)benzamide (Compound 761)

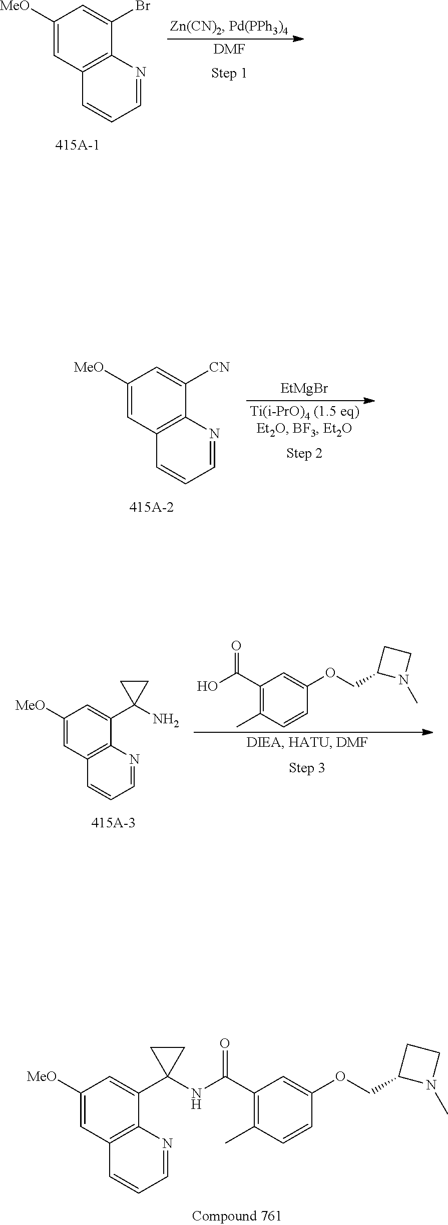

### Step 1:6-Methoxyquinoline-8-carbonitrile (415A-2)

To a solution of 8-bromo-6-methoxyquinoline (13.0 g, 54.6 mmol, 1.0 eq) in DMF (250 mL) were added Zn(CN)₂ (12.8 mg, 109 mmol, 6.93 mL, 2.0 eq) and Pd(PPh₃)₄ (6.31 g, 5.46 mmol, 0.1 eq) under a N₂ atmosphere. The mixture was stirred at 100° C. for 15 h. LCMS indicated that the starting material was completely consumed, and the desired mass was detected. The reaction mixture was allowed to cool to room temperature, poured into H₂O (400 mL), and extracted with EtOAc (200 mL×3). The combined organic layers were dried over Na₂SO₄, filtered, and concentrated under vacuum to give a residue which was purified by flash silica gel chromatography using a gradient of EtOAc/petroleum ether from 0/1 to 1/9. 6-Methoxyquinoline-8-carbonitrile (9.00 g, 48.9 mmol, 89% yield) was obtained as a white solid. M+H⁺=185.0 (LCMS).

### Step 2: 1-(6-Methoxyquinolin-8-yl)cyclopropan-1-amine (415A-3)

A mixture of 6-methoxyquinoline-8-carbonitrile (1.00 g, 5.43 mmol, 1.0 eq) in anhydrous Et₂O (100 mL) was degassed and purged with N₂ three times. The mixture was cooled to −78° C. To this mixture was added Ti(i-PrO)₄ (2.31 g, 8.14 mmol, 2.4 mL, 15 eq) slowly, then EtMgBr (3 M in Et₂O, 4.0 mL, 2.2 eq) was added dropwise to maintain the temperature between −78° C. and −75° C. over 1 h under a N₂ atmosphere. After the addition was complete, the resulting mixture was stirred at the same temperature for 10 min and warmed to 20° C. over 1 h. BF₃·Et₂O (1.54 g, 10.9 mmol, 1.34 mL, 2.0 eq) was added slowly with no obvious temperature change. The mixture was stirred at 20° C. for another 1 h. TLC indicated that the starting material was completely consumed, and a main spot was detected. The reaction mixture was added into a mixture of HCl (1 M aqueous) (50 mL) and MTBE (50 mL) and extracted with MTBE (50 mL×2). The aqueous layer was basified to pH 8 by using NaOH (2 M aqueous), and a precipitate was formed. The mixture was filtered through a pad of Celite and the slurry was washed with DCM several times. The combined filtrate was extracted with DCM (100 mL×6). The combined organic layers were dried over Na₂SO₄, filtered, and concentrated under vacuum to give a residue which was purified by flash silica gel chromatography using a gradient of DCM/MeOH from 0/1 to 10/1. 1-(6-Methoxyquinolin-8-yl)cyclopropan-1-amine (370 mg, 1.73 mmol, 32% yield) was obtained as a yellow oil. M+H⁺=215.2 (LCMS).

### Step 3: (S)—N-(1-(6-Methoxyquinolin-8-yl)cyclopropyl)-2-methyl-5-((1-methylazetidin-2-yl)methoxy)benzamide (Compound 761)

To a mixture of 1-(6-methoxyquinolin-8-yl)cyclopropan-1-amine (100 mg, 467 μmol, 1.0 eq) and(S)-2-methyl-5-((1-methylazetidin-2-yl)methoxy)benzoic acid (110 mg, 467 μmol, 1.0 eq) in DMF (3.0 mL) were added HATU (444 mg, 1.17 mmol, 2.5 eq) and DIEA (181 mg, 1.40 mmol, 244 μL, 3.0 eq). The resulting mixture was stirred at 20° C. for 15 h. LCMS indicated that the starting material was completely consumed, and the desired mass was detected. The reaction mixture was poured into H₂O (15 mL) and extracted with EtOAc (15 mL×4). The combined organic layers were dried over Na₂SO₄, filtered, and concentrated under vacuum to give a residue which was purified by preparative HPLC (Phenomenex Luna C18 column (100× 40 mm, 10 μm); flow rate: 60 mL/min; gradient: 1%-25% B over 8 min; mobile phase A: 0.1% aqueous TFA, mobile phase B: acetonitrile). (S)—N-(1-(6-Methoxyquinolin-8-yl)cyclopropyl)-2-methyl-5-((1-methylazetidin-2-yl)methoxy)benzamide (42.7 mg, 75.9 μmol, 16% yield) was obtained as a white solid. M+H⁺=432.1 (LCMS); ¹H NMR (400 MHZ, DMSO-d₆) δ 10.54-9.94 (m, 1H), 9.19 (s, 1H), 9.07-8.96 (m, 1H), 8.77 (br d, J=8.3 Hz, 1H), 7.73-7.58 (m, 2H), 7.23 (d, J=8.4 Hz, 1H), 7.16-6.95 (m, 2H), 6.88 (s, 1H), 4.91-4.65 (m, 1H), 4.52-4.28 (m, 2H), 4.21-4.02 (m, 5H), 2.96-2.86 (m, 3H), 2.47-2.38 (m, 2H), 2.32 (s, 3H), 1.63-1.54 (m, 2H), 1.49-1.42 (m, 2H).

### Example 416: 5-(2-(tert-Butylamino)ethoxy)-N-(1-(7-methoxyquinolin-5-yl)cyclopropyl)-2-methylbenzamide (Compound 626)

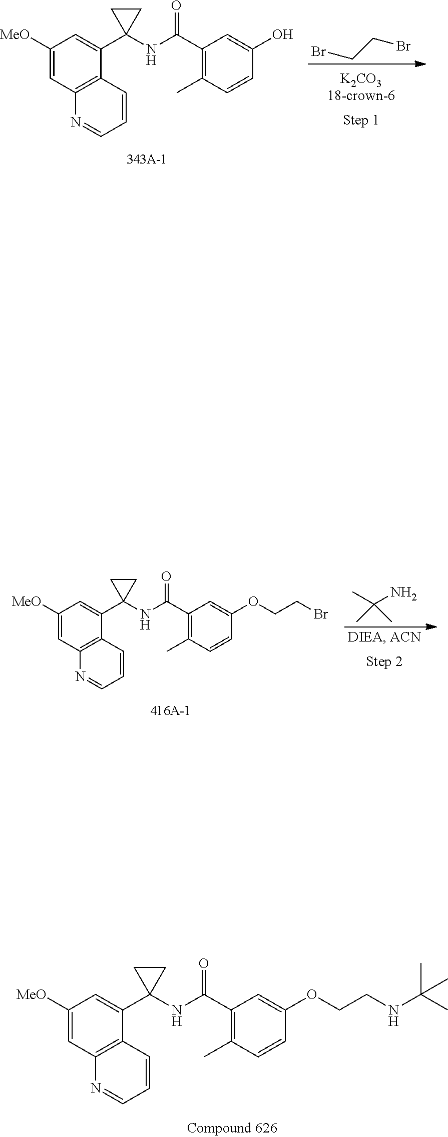

### Step 1: 5-(2-Bromoethoxy)-N-(1-(7-methoxyquinolin-5-yl)cyclopropyl)-2-methylbenzamide (416A-1)

To a solution of 5-hydroxy-N-(1-(7-methoxyquinolin-5-yl)cyclopropyl)-2-methylbenzamide (150 mg, 431 μmol, 1.0 eq) and 1,2-dibromoethane (809 mg, 4.31 mmol, 325 μL, 10 eq) in acetone (5.0 mL) were added K₂CO₃ (137 mg, 990 μmol, 2.3 eq) and 18-crown-6 (5.69 mg, 21.5 μmol, 0.05 eq). The mixture was stirred at 60° C. for 16 h. LCMS indicated that the starting material was completely consumed, and the desired mass was detected. The reaction mixture was allowed to cool to room temperature and concentrated under vacuum to give a residue which was purified by preparative TLC (EtOAc/petroleum ether=1/3, R_{f}=0.38). 5-(2-Bromoethoxy)-N-(1-(7-methoxyquinolin-5-yl)cyclopropyl)-2-methylbenzamide (63.0 mg, 138 μmol, 32% yield) was obtained as a yellow solid. M+H⁺=455.1 (LCMS).

### Step 2: 5-(2-(tert-Butylamino)ethoxy)-N-(1-(7-methoxyquinolin-5-yl)cyclopropyl)-2-methylbenzamide (Compound 626)

To a solution of 2-methylpropan-2-amine (101 mg, 1.38 mmol, 10 eq) in acetonitrile (5.0 mL) were added 5-(2-bromoethoxy)-N-(1-(7-methoxyquinolin-5-yl)cyclopropyl)-2-methylbenzamide (63.0 mg, 138 μmol, 1.0 eq) and DIEA (53.7 mg, 415 μmol, 72.3 μL, 3.0 eq). The mixture was stirred at 20° C. for 16 h. LCMS indicated that the starting material was completely consumed, and the desired mass was detected. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (column: Waters Xbridge BEH C18 (100×30 mm, 10 μm); flow rate: 60 mL/min; gradient: 1%-30% B over 8 min; mobile phase A: 0.04% aqueous HCl, mobile phase B: acetonitrile). 5-(2-(tert-Butylamino)ethoxy)-N-(1-(7-methoxyquinolin-5-yl)cyclopropyl)-2-methylbenzamide (19.2 mg, 39.2 μmol, 99% yield, HCl salt) was obtained as a yellow solid. M+H⁺=448.2 (LCMS); ¹H NMR (400 MHZ, DMSO-d₆) δ 9.62 (br d, J=8.1 Hz, 1H), 9.33 (s, 1H), 9.16 (d, J=5.0 Hz, 1H), 8.97 (br s, 2H), 7.94 (br dd, J=5.4, 8.3 Hz, 1H), 7.71 (d, J=2.4 Hz, 1H), 7.61 (s, 1H), 7.10 (d, J=8.6 Hz, 1H), 6.92 (dd, J=2.6, 8.4 Hz, 1H), 6.75 (d, J=2.8 Hz, 1H), 4.21 (br t, J=5.1 Hz, 2H), 4.02 (s, 3H), 3.22 (br s, 2H), 1.97 (s, 3H), 1.41 (br s, 2H), 1.31 (s, 11H).

### Example 417: (S)—N-(1-(7-Methoxyquinolin-5-yl)cyclopropyl)-2-methyl-5-((1-methylazetidin-2-yl)methoxy-d₂)benzamide (Compound 797)

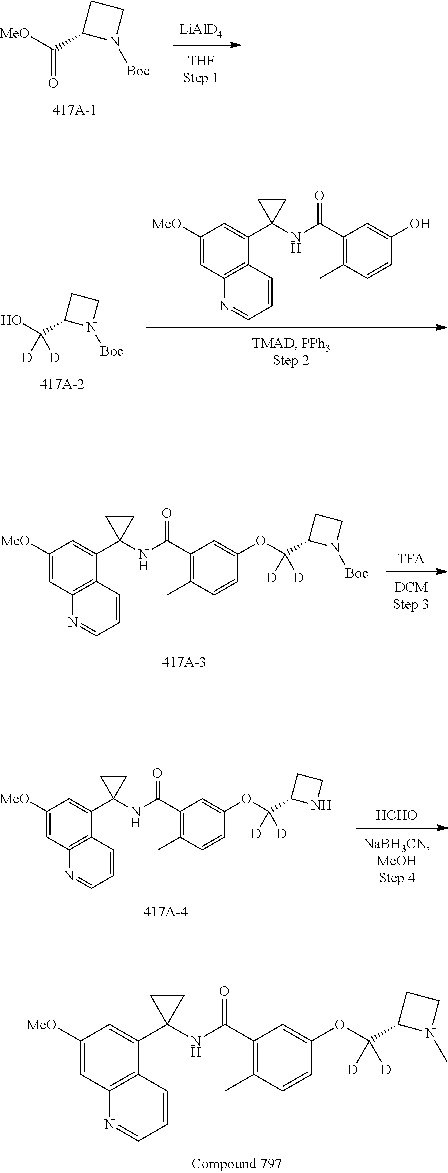

### Step 1: tert-Butyl(S)-2-(hydroxymethyl-d₂)azetidine-1-carboxylate (417A-2)

To a solution of 1-(tert-butyl) 2-methyl(S)-azetidine-1,2-dicarboxylate (400 mg, 1.86 mmol, 1.0 eq) in THF (5.0 mL) was added LiAlD₄ (77.9 mg, 1.86 mmol, 106 μL, 1.0 eq) at 0° C. The mixture was stirred at 25° C. for 1 h under a N₂ atmosphere. LCMS indicated that the starting material was completely consumed, and the desired mass was detected. The mixture was treated with saturated NH₄Cl aqueous (10 mL) and extracted with EtOAc (5.0 mL×3). The combined organic layers were dried over Na₂SO₄, filtered, and concentrated under vacuum to give tert-butyl(S)-2-(hydroxymethyl-d₂)azetidine-1-carboxylate (300 mg, 1.59 mmol, 85% yield) as a yellow oil, which was used in the next step without any further purification. M−56+H⁺=134.1. 1H NMR (400 MHZ, CDCl₃) δ 4.44 (t, J=7.6 Hz, 1H), 3.93-3.84 (m, 1H), 3.79 (dt, J=4.8, 8.9 Hz, 1H), 2.18 (dtd, J=4.8, 8.7, 11.4 Hz, 1H), 1.94 (tdd, J=7.0, 9.3, 11.3 Hz, 1H), 1.46 (s, 9H).

### Step 2: tert-Butyl(S)-2-((3-((1-(7-methoxyquinolin-5-yl)cyclopropyl)carbamoyl)-4-methylphenoxy)methyl-d₂)azetidine-1-carboxylate (417A-3)

To a solution of tert-butyl(S)-2-(hydroxymethyl-d₂)azetidine-1-carboxylate (166 mg, 878 μmol, 1.7 eq) and 5-hydroxy-N-(1-(7-methoxyquinolin-5-yl)cyclopropyl)-2-methylbenzamide (180 mg, 517 μmol, 1.0 eq) in toluene (3.0 mL) were added added TMAD (267 mg, 1.55 mmol, 3.0 eq) and PPh₃ (407 mg, 1.55 mmol, 3.0 eq) in one portion. The mixture was stirred at 100° C. for 16 h under a N₂ atmosphere. LCMS indicated that the starting material was completely consumed, and the desired mass was detected. The reaction mixture was allowed to cool to room temperature, poured into H₂O (10 mL), and extracted with EtOAc (10 mL×3). The combined organic layers were dried over Na₂SO₄, filtered, and concentrated under vacuum to give tert-butyl(S)-2-((3-((1-(7-methoxyquinolin-5-yl)cyclopropyl)carbamoyl)-4-methylphenoxy)methyl-d₂)azetidine-1-carboxylate (260 mg) as a yellow solid, which was used in the next step without any further purification. M+H⁺=520.3 (LCMS).

### Step 3: (S)-5-(Azetidin-2-ylmethoxy-d₂)—N-(1-(7-methoxyquinolin-5-yl)cyclopropyl)-2-methylbenzamide (417A-4)

To a solution of tert-butyl(S)-2-((3-((1-(7-methoxyquinolin-5-yl)cyclopropyl)carbamoyl)-4-methylphenoxy)methyl-d₂)azetidine-1-carboxylate (260 mg, 450 μmol, 1.0 eq) in DCM (1.0 mL) was added TFA (250 μL) at 0° C. The mixture was stirred at 25° C. for 2 h. LCMS indicated that the starting material was completely consumed, and the desired mass was detected. The mixture was concentrated under vacuum at 25° C. to give(S)-5-(azetidin-2-ylmethoxy-d₂)—N-(1-(7-methoxyquinolin-5-yl)cyclopropyl)-2-methylbenzamide (180 mg, 429 μmol, 95% yield, TFA salt) as a yellow solid, which was used in the next step without any further purification.

### Step 4: (S)—N-(1-(7-Methoxyquinolin-5-yl)cyclopropyl)-2-methyl-5-((1-methylazetidin-2-yl)methoxy-d₂)benzamide (Compound 797)

To a solution of(S)-5-(azetidin-2-ylmethoxy-d₂)—N-(1-(7-methoxyquinolin-5-yl)cyclopropyl)-2-methylbenzamide (180 mg, 337 mmol, 1.0 eq, TFA) in MeOH (3.0 mL) was added TEA (50.0 μL), followed by formaldehyde (54.8 mg, 675 μmol, 50.2 μL, 37% purity in water, 2.0 eq). The mixture was adjusted to pH 5 with a small amount of AcOH, then NaBH₃CN (42.4 mg, 675 μmol, 2.0 eq) was added. The resulting mixture was stirred at 20° C. for 2 h. LCMS indicated that the starting material was completely consumed, and the desired mass was detected. The reaction mixture was poured into saturated aqueous NH₄Cl (10 mL) and extracted with EtOAc (10 mL×3). The combined organic layers were dried over Na₂SO₄, filtered, and concentrated under vacuum to give a residue, which was purified by preparative HPLC (Phenomenex Luna C18 column (100×30 mm, 5 μm); flow rate: 25 mL/min; gradient: 5%-35% B over 8 min; mobile phase A: 0.1% aqueous TFA, mobile phase B: acetonitrile). (S)—N-(1-(7-Methoxyquinolin-5-yl)cyclopropyl)-2-methyl-5-((1-methylazetidin-2-yl)methoxy-d₂)benzamide (77.6 mg, 179 μmol, 53% yield, TFA salt) was obtained as a white solid. M+H⁺=434.2 (LCMS); ¹H NMR (400 MHZ, DMSO-d₆) δ 9.31 (br d, J=8.5 Hz, 1H), 9.22-9.16 (m, 1H), 9.01 (dd, J=1.3, 4.9 Hz, 1H), 7.72 (dd, J=4.9, 8.4 Hz, 1H), 7.62 (d, J=2.4 Hz, 1H), 7.41 (d, J=2.4 Hz, 1H), 7.12-7.07 (m, 1H), 6.95-6.88 (m, 1H), 6.77-6.70 (m, 1H), 4.65-4.55 (m, 1H), 4.07-4.00 (m, 1H), 3.97 (s, 3H), 3.87 (q, J=9.5 Hz, 1H), 2.83 (s, 3H), 2.40-2.28 (m, 2H), 1.94 (s, 3H), 1.37 (br s, 2H), 1.26 (br s, 2H).

### Example 418: (S)—N-(1-(7-Methoxyquinolin-5-yl)cyclopropyl)-2-methyl-5-((1-(methyl-d₃)azetidin-2-yl)methoxy)benzamide (Compound 732)

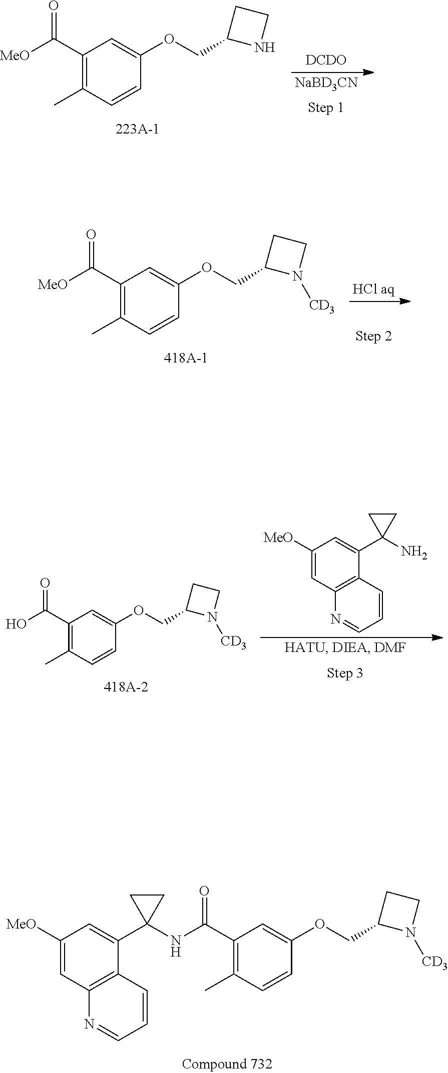

### Step 1: Methyl(S)-2-methyl-5-((1-(methyl-d₃)azetidin-2-yl)methoxy)benzoate (418A-1)

To a solution of(S)-methyl 5-(azetidin-2-ylmethoxy)-2-methylbenzoate (200 mg, 573 μmol, 1.0 eq, TFA salt) in MeOH (4.0 mL) was added TEA (100 μL), followed by formaldehyde-d₂ (107 mg, 687 μmol, 94.6 μL, 20% purity in D₂O, 1.2 eq). The resulting mixture was adjusted to pH 6 with a small amount of AcOH. The mixture was stirred at 20° C. for 30 min, then NaBD₃CN (36.0 mg, 573 μmol, 1.0 eq) was added. The resulting mixture was stirred at 20° C. for another 1 h. LCMS indicated that the starting material was completely consumed, and the desired mass was detected. The reaction mixture was poured into H₂O (10 mL) and extracted with DCM (20 mL×5). The combined organic layers were dried over Na₂SO₄, filtered, and concentrated under vacuum to give a residue which was purified by flash silica gel chromatography using a gradient of DCM/MeOH from 100/1 to 10/1. Methyl(S)-2-methyl-5-((1-(methyl-d₃)azetidin-2-yl)methoxy)benzoate (80.0 mg, 317 μmol, 55% yield) was obtained as a yellow oil. M+H⁺=253.1 (LCMS).

### Step 2: (S)-2-Methyl-5-((1-(methyl-d₃)azetidin-2-yl)methoxy)benzoic acid (418A-2)

A solution of methyl(S)-2-methyl-5-((1-(methyl-d₃)azetidin-2-yl)methoxy)benzoate (180 mg, 713 μmol, 1.0 eq) in HCl (2 M aqueous, 10 mL) was stirred at 100° C. for 16 h. LCMS indicated that the starting material was completely consumed, and the desired mass was detected. The reaction mixture was allowed to cool to room temperature, then adjusted to pH 6 with NaOH (2 M aqueous). The resulting mixture was concentrated under vacuum to remove the water completely. The resulting mixture was treated with MeOH/DCM (V/V=10/1, 10 mL), then filtered. The filter cake was washed with MeOH/DCM (V/V=10/1, 10 mL×2) to ensure all product was washed from the solids. The combined organic layers were concentrated under vacuum to give(S)-2-methyl-5-((1-(methyl-d₃)azetidin-2-yl)methoxy)benzoic acid (190 mg, crude) as a hydrophilic, white solid. M+H⁺=239.2 (LCMS); ¹H NMR (400 MHZ, DMSO-d₆) δ 11.00-10.70 (m, 1H), 7.49-7.44 (m, 1H), 7.30 (d, J=8.4 Hz, 1H), 7.16 (dd, J=2.8, 8.4 Hz, 1H), 4.79-4.65 (m, 1H), 4.54-4.47 (m, 1H), 4.40-4.33 (m, 1H), 4.14-4.02 (m, 1H), 3.97-3.84 (m, 1H), 2.56-2.55 (m, 3H), 2.47-2.37 (m, 2H).

### Step 3: (S)—N-(1-(7-Methoxyquinolin-5-yl)cyclopropyl)-2-methyl-5-((1-(methyl-d₃)azetidin-2-yl)methoxy)benzamide (Compound 732)

To a solution of(S)-2-methyl-5-((1-(methyl-d₃)azetidin-2-yl)methoxy)benzoic acid (190 mg, 797 μmol, 1.0 eq) and 1-(7-methoxyquinolin-5-yl)cyclopropan-1-amine (188 mg, 877 μmol, 1.1 eq) in DMF (10 mL) were added DIEA (309 mg, 2.39 mmol, 417 μL, 3.0 eq) and HATU (606 mg, 1.59 mmol, 2.0 eq). The mixture was stirred at 20° C. for 4 h. LCMS indicated that the starting material was completely consumed, and the desired mass was detected. The reaction mixture was poured into H₂O (20 mL) and extracted with EtOAc (20 mL×6). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 25%-55% B over 8 min; mobile phase A: 10 mM aqueous NH₄HCO₃, mobile phase B: acetonitrile). (S)—N-(1-(7-Methoxyquinolin-5-yl)cyclopropyl)-2-methyl-5-((1-(methyl-d₃)azetidin-2-yl)methoxy)benzamide (52.3 mg, 120 μmol, 15% yield) was obtained as a white solid. M+H⁺=435.4 (LCMS); ¹H NMR (400 MHZ, DMSO-d₆) δ 9.10 (s, 1H), 9.00-8.90 (m, 1H), 8.86-8.79 (m, 1H), 7.48-7.39 (m, 2H), 7.31 (d, J=2.4 Hz, 1H), 7.03 (d, J=8.4 Hz, 1H), 6.84 (dd, J=2.8, 8.4 Hz, 1H), 6.61 (d, J=2.7 Hz, 1H), 3.94-3.91 (m, 3H), 3.88-3.84 (m, 2H), 3.28-3.16 (m, 2H), 2.76-2.64 (m, 1H), 2.00-1.80 (m, 5H), 1.37-1.31 (m, 2H), 1.22-1.16 (m, 2H).

### Example 419: tert-Butyl(6S,8aR)-6-((3-((1-(7-methoxyquinolin-5-yl)cyclopropyl)carbamoyl)-4-methylphenoxy)methyl) hexahydropyrrolo[1,2-a]pyrazine-2(1H)-carboxylate (Compound 775) and tert-butyl(7S,9aR)-7-(3-((1-(7-methoxyquinolin-5-yl)cyclopropyl)carbamoyl)-4-methylphenoxy) octahydro-2H-pyrido[1,2-a]pyrazine-2-carboxylate (Compound 776)

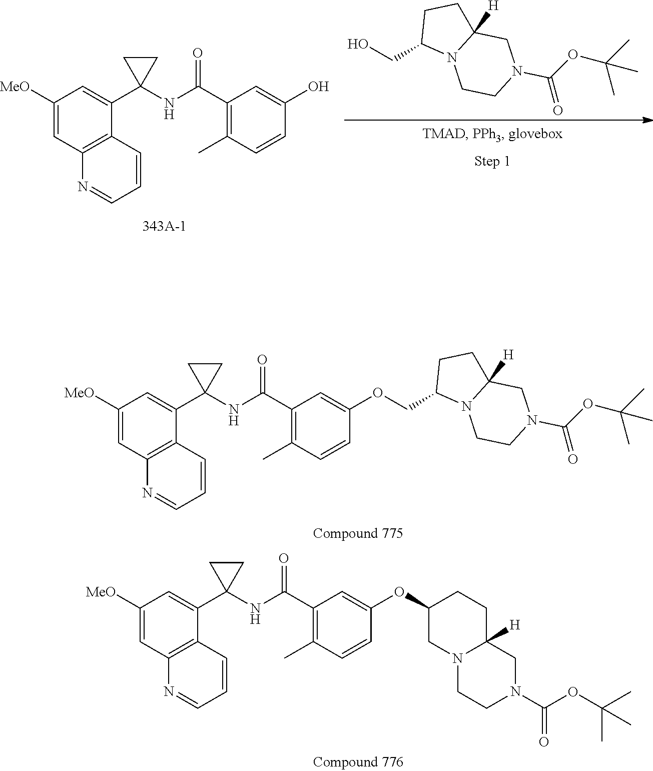 [SMILES: COc1cc(C2(NC(=O)c3cc(O)ccc3C)CC2)c2cccnc2c1 ; CC(C)(C)OC(=O)N1CCN2[C@H](CO)CC[C@@H]2C1 ; COc1cc(C2(NC(=O)c3cc(OC[C@@H]4CC[C@@H]5CN(C(=O)OC(C)(C)C)CCN54)ccc3C)CC2)c2cccnc2c1 ; COc1cc(C2(NC(=O)c3cc(O[C@H]4CC[C@@H]5CN(C(=O)OC(C)(C)C)CCN5C4)ccc3C)CC2)c2cccnc2c1]

### Step 1: tert-Butyl(6S,8aR)-6-((3-((1-(7-methoxyquinolin-5-yl)cyclopropyl)carbamoyl)-4-methylphenoxy)methyl) hexahydropyrrolo[1,2-a]pyrazine-2(1H)-carboxylate (Compound 775) and tert-butyl(7S,9aR)-7-(3-((1-(7-methoxyquinolin-5-yl)cyclopropyl)carbamoyl)-4-methylphenoxy) octahydro-2H-pyrido[1,2-a]pyrazine-2-carboxylate (Compound 776)

To a mixture of 5-hydroxy-N-(1-(7-methoxyquinolin-5-yl)cyclopropyl)-2-methylbenzamide (150 mg, 431 μmol, 1.0 eq) and tert-butyl(6S,8aR)-6-(hydroxymethyl)hexahydropyrrolo[1,2-a]pyrazine-2(1H)-carboxylate (165 mg, 645 μmol, 1.5 eq) in toluene (15 mL) was added CMBP (312 mg, 1.29 mmol, 3.0 eq) in glove box. The mixture was degassed and purged with N₂ three times. The mixture was stirred at 100° C. for 12 h under a N₂ atmosphere. LCMS indicated that the starting material was completely consumed. The reaction mixture was allowed to cool to room temperature, poured into H₂O (10 mL), and extracted with EtOAc (5.0 mL×2). The combined organic layers were dried over Na₂SO₄, filtered, and concentrated under vacuum to give a residue which was purified by preparative HPLC (Waters Xbridge BEH C18 column (100×30 mm, 10 μm); flow rate: 60 mL/min; gradient: 30%-60% B over 8 min; mobile phase A: 10 mM aqueous NH₄HCO₃, mobile phase B: acetonitrile). tert-Butyl(6S,8aR)-6-((3-((1-(7-methoxyquinolin-5-yl)cyclopropyl)carbamoyl)-4-methylphenoxy)methyl) hexa hydropyrrolo[1,2-a]pyrazine-2(1H)-carboxylate (183 mg, 311 μmol, 72% yield, eluent second) was obtained as a white solid. M+H⁺=587.3 (LCMS); ¹H NMR (400 MHZ, DMSO-d₆) δ 9.09 (s, 1H), 8.94 (d, J=8.4 Hz, 1H), 8.81 (d, J=3.3 Hz, 1H), 7.51-7.35 (m, 2H), 7.31 (d, J=2.4 Hz, 1H), 7.03 (d, J=8.5 Hz, 1H), 6.84 (dd, J=2.5, 8.4 Hz, 1H), 6.62 (d, J=2.5 Hz, 1H), 4.06-3.95 (m, 1H), 3.92 (s, 3H), 3.85-3.79 (m, 1H), 3.13 (br d, J=11.1 Hz, 1H), 2.83-2.53 (m, 3H), 2.05-1.91 (m, 5H), 1.90-1.80 (m, 1H), 1.78-1.66 (m, 1H), 1.61-1.12 (m, 17H). tert-Butyl(7S,9aR)-7-(3-((1-(7-methoxyquinolin-5-yl)cyclopropyl)carbamoyl)-4-methylphen oxy) octahydro-2H-pyrido[1,2-a]pyrazine-2-carboxylate (9.20 mg, 15.7 μmol, 11% yield, eluent first) was obtained as a white solid. M+H⁺=587.3 (LCMS); ¹H NMR (400 MHz, DMSO-d₆) δ 9.11 (s, 1H), 8.95 (d, J=7.5 Hz, 1H), 8.82 (dd, J=1.6, 4.1 Hz, 1H), 7.51-7.37 (m, 2H), 7.31 (d, J=2.5 Hz, 1H), 7.03 (d, J=8.5 Hz, 1H), 6.85 (dd, J=2.6, 8.4 Hz, 1H), 6.61 (d, J=2.6 Hz, 1H), 4.50-4.40 (m, 1H), 3.92 (s, 3H), 3.84-3.69 (m, 2H), 2.91 (br d, J=12.5 Hz, 1H), 2.57 (br s, 1H), 2.15 (br d, J=11.9 Hz, 1H), 1.96-1.76 (m, 6H), 1.55-1.31 (m, 16H), 1.22-1.14 (m, 2H).

### Example 420: N-(1-(7-Methoxyquinolin-5-yl)cyclopropyl)-2-methyl-5-(((6S,8aR)-octahydropyrrolo[1,2-a]pyrazin-6-yl)methoxy)benzamide (Compound 789) and N-(1-(7-methoxyquinolin-5-yl)cyclopropyl)-2-methyl-5-(((7S,9aR)-octahydro-2H-pyrido[1,2-a]pyrazin-7-yl)oxy)benzamide (Compound 790)

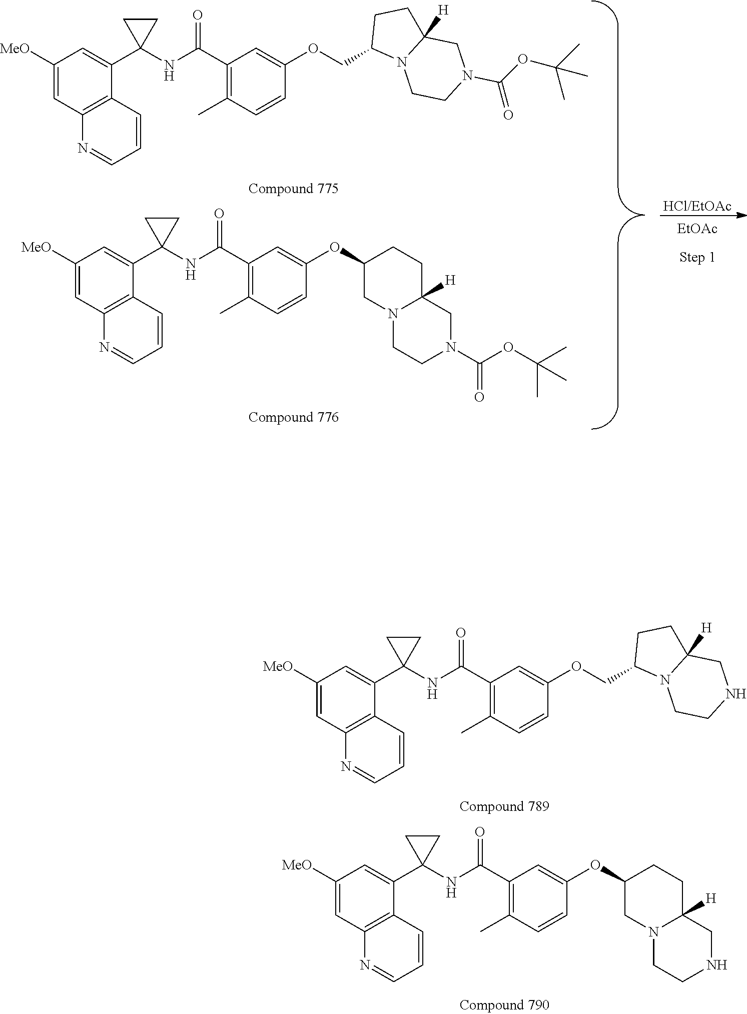 [SMILES: "EtOAc" ; COc1cc(C2(NC(=O)c3cc(OC[C@@H]4CC[C@@H]5CN(C(=O)OC(C)(C)C)CCN54)ccc3C)CC2)c2cccnc2c1 ; "HCl/EtOAc" ; COc1cc(C2(NC(=O)c3cc(O[C@H]4CC[C@@H]5CN(C(=O)OC(C)(C)C)CCN5C4)ccc3C)CC2)c2cccnc2c1 ; COc1cc(C2(NC(=O)c3cc(OC[C@@H]4CC[C@@H]5CNCCN54)ccc3C)CC2)c2cccnc2c1 ; COc1cc(C2(NC(=O)c3cc(O[C@H]4CC[C@@H]5CNCCN5C4)ccc3C)CC2)c2cccnc2c1]

### Step 1: N-(1-(7-Methoxyquinolin-5-yl)cyclopropyl)-2-methyl-5-(((6S,8aR)-octahydropyrrolo[1,2-a]pyrazin-6-yl)methoxy)benzamide (Compound 789) and N-(1-(7-methoxyquinolin-5-yl)cyclopropyl)-2-methyl-5-(((7S,9aR)-octahydro-2H-pyrido[1,2-a]pyrazin-7-yl)oxy)benzamide (Compound 790)

To a mixture of tert-butyl(6S,8aR)-6-((3-((1-(7-methoxyquinolin-5-yl)cyclopropyl) carbamoyl)-4-methylphenoxy)methyl) hexahydropyrrolo[1,2-a]pyrazine-2(1H)-carboxylate and tert-butyl(7S,9aR)-7-(3-((1-(7-methoxyquinolin-5-yl)cyclopropyl)carbamoyl)-4-methylphenoxy) octahydro-2H-pyrido[1,2-a]pyrazine-2-carboxylate (70.0 mg, 119 μmol, 1.0 eq) in EtOAc (1.0 mL) was added HCl/EtOAc (4 M, 2.19 mL). The resulting mixture was stirred at 20° C. for 30 min. LCMS indicated that the starting material was completely consumed. The reaction mixture was concentrated under vacuum at 20° C. to give a residue which was purified by preparative HPLC (Phenomenex Luna C18 column (80×30 mm, 3 μm); flow rate: 25 mL/min; gradient: 5%-25% B over 8 min; mobile phase A: 0.04% aqueous HCl, mobile phase B: acetonitrile). N-(1-(7-Methoxyquinolin-5-yl)cyclopropyl)-2-methyl-5-(((6S,8aR)-octahydropyrrolo[1,2-a]pyrazin-6-yl)methoxy)benzamide (26.8 mg, 51.2 μmol, 43% yield, HCl salt, eluent second) was obtained as a yellow solid. M+H⁺=487.3 (LCMS); ¹H NMR (400 MHZ, DMSO-d₆) δ 9.62 (d, J=8.4 Hz, 1H), 9.30 (s, 1H), 9.18-9.08 (m, 1H), 7.94 (dd, J=5.4, 8.5 Hz, 1H), 7.72 (d, J=2.4 Hz, 1H), 7.52 (d, J=2.3 Hz, 1H), 7.08 (d, J=8.6 Hz, 1H), 6.92 (dd, J=2.6, 8.4 Hz, 1H), 6.74 (d, J=2.6 Hz, 1H), 4.40-4.14 (m, 2H), 4.01 (s, 3H), 3.96-3.82 (m, 1H), 3.77-3.62 (m, 2H), 3.48-3.16 (m, 5H), 2.28-2.05 (m, 2H), 1.93 (s, 3H), 1.81-1.59 (m, 2H), 1.41 (br s, 2H), 1.30 (br d, J=3.4 Hz, 2H). N-(1-(7-Methoxyquinolin-5-yl)cyclopropyl)-2-methyl-5-(((7S,9aR)-octahydro-2H-pyrido[1,2-a]pyrazin-7-yl)oxy)benzamide (11.7 mg, 22.4 μmol, 19% yield, HCl salt, eluent first) was obtained as a yellow solid. M+H⁺=487.3 (LCMS); ¹H NMR (400 MHZ, DMSO-d₆) δ 9.54 (d, J=8.4 Hz, 1H), 9.26 (s, 1H), 9.10 (d, J=5.1 Hz, 1H), 7.88 (dd, J=5.4, 8.5 Hz, 1H), 7.69 (d, J=2.4 Hz, 1H), 7.47 (d, J=2.3 Hz, 1H), 7.09 (d, J=8.6 Hz, 1H), 7.02-6.92 (m, 1H), 6.80 (d, J=2.4 Hz, 1H), 4.74 (br s, 1H), 4.00 (s, 3H), 3.53-3.21 (m, 7H), 3.20-3.03 (m, 2H), 1.97-1.85 (m, 4H), 1.82-1.57 (m, 3H), 1.39 (br s, 2H), 1.29 (br s, 2H).

### Example 421: N-(1-(7-Methoxyquinolin-5-yl)cyclopropyl)-2-methyl-5-(((6S,8aR)-2-methyloctahydropyrrolo[1,2-a]pyrazin-6-yl)methoxy)benzamide (Compound 784) and N-(1-(7-methoxyquinolin-5-yl)cyclopropyl)-2-methyl-5-(((7S,9aR)-2-methyloctahydro-2H-pyrido[1,2-a]pyrazin-7-yl)oxy)benzamide (Compound 783)

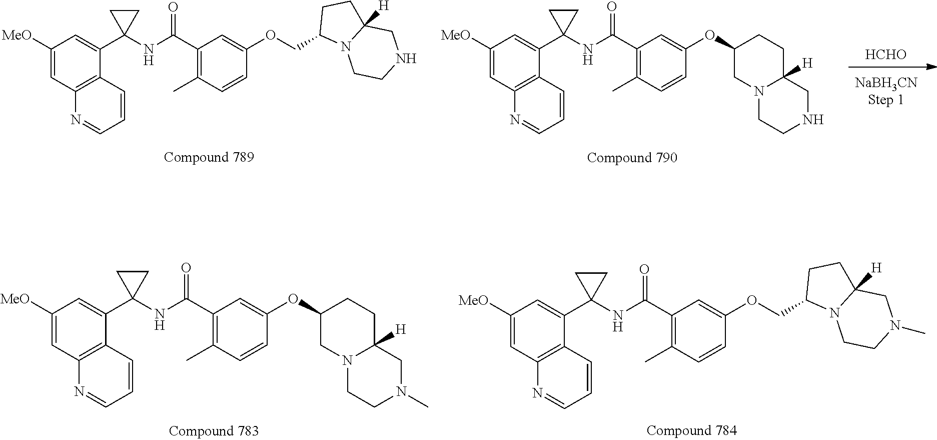 [SMILES: COc1cc(C2(NC(=O)c3cc(OC[C@@H]4CC[C@@H]5CNCCN54)ccc3C)CC2)c2cccnc2c1 ; COc1cc(C2(NC(=O)c3cc(O[C@H]4CC[C@@H]5CNCCN5C4)ccc3C)CC2)c2cccnc2c1 ; "HCHO" ; COc1cc(C2(NC(=O)c3cc(O[C@H]4CC[C@@H]5CN(C)CCN5C4)ccc3C)CC2)c2cccnc2c1 ; COc1cc(C2(NC(=O)c3cc(OC[C@@H]4CC[C@@H]5CN(C)CCN54)ccc3C)CC2)c2cccnc2c1]

### Step 1: N-(1-(7-Methoxyquinolin-5-yl)cyclopropyl)-2-methyl-5-(((7S,9aR)-2-methyl octahydro-2H-pyrido[1,2-a]pyrazin-7-yl)oxy)benzamide (Compound 783) and N-(1-(7-methoxyquinolin-5-yl)cyclopropyl)-2-methyl-5-(((6S,8aR)-2-methyloctahydropyrrolo[1,2-a]pyrazin-6-yl)methoxy)benzamide (Compound 784)

To a mixture of N-(1-(7-methoxyquinolin-5-yl)cyclopropyl)-2-methyl-5-(((6S,8aR)-octahydropyrrolo[1,2-a]pyrazin-6-yl)methoxy)benzamide and N-(1-(7-methoxyquinolin-5-yl)cyclopropyl)-2-methyl-5-(((7S,9aR)-octahydro-2H-pyrido[1,2-a]pyrazin-7-yl)oxy)benzamide (130 mg, 249 μmol, 1.0 eq, HCl salt) in MeOH (5.0 mL) was added TEA (50 L), followed by formaldehyde (40.3 mg, 497 μmol, 37.0 μL, 37% purity in water, 2.0 eq). The resulting mixture was adjusted to pH 6 with a small amount of AcOH, then NaBH₃CN (31.2 mg, 497 μmol, 2.0 eq) was added. The mixture was stirred at 20° C. for 16 h. LCMS indicated that the starting material was completely consumed, and the desired mass was detected. The reaction mixture was poured into H₂O (5.0 mL) and extracted with EtOAc (5.0 mL×3). The combined organic layers were dried over Na₂SO₄, filtered, and concentrated under vacuum to give a residue which was purified by preparative HPLC (Waters Xbridge BEH C18 column (100×30 mm, 10 μm); flow rate: 60 mL/min; gradient: 25%-55% B over 8 min; mobile phase A: 10 mM aqueous NH₄HCO₃, mobile phase B: acetonitrile). N-(1-(7-methoxyquinolin-5-yl)cyclopropyl)-2-methyl-5-(((6S,8aR)-2-methyloctahydropyrrolo[1,2-a]pyrazin-6-yl)methoxy)benzamide (16.8 mg, 32.8 μmol, 13% yield, eluent second) was obtained as a white solid. M+H⁺=501.3 (LCMS); ¹H NMR (400 MHZ, DMSO-d₆) δ 9.08 (s, 1H), 9.00-8.87 (m, 1H), 8.81 (dd, J=1.6, 4.3 Hz, 1H), 7.46 (d, J=2.6 Hz, 1H), 7.41 (dd, J=4.3, 8.4 Hz, 1H), 7.31 (d, J=2.5 Hz, 1H), 7.02 (d, J=8.5 Hz, 1H), 6.83 (dd, J=2.8, 8.4 Hz, 1H), 6.61 (d, J=2.8 Hz, 1H), 3.95-3.87 (m, 4H), 3.78 (dd, J=5.4, 9.7 Hz, 1H), 3.10-3.01 (m, 1H), 2.81 (br d, J=10.3 Hz, 1H), 2.69-2.58 (m, 2H), 2.26-2.09 (m, 5H), 2.02-1.79 (m, 5H), 1.74-1.59 (m, 2H), 1.54-1.38 (m, 1H), 1.38-1.30 (m, 2H), 1.28-1.14 (m, 3H). N-(1-(7-Methoxyquinolin-5-yl)cyclopropyl)-2-methyl-5-(((7S,9aR)-2-methyloctahydro-2H-pyrido[1,2-a]pyrazin-7-yl)oxy)benzamide (10.2 mg, 19.3 μmol, 8% yield, eluent first) was obtained as a white solid. M+H⁺=501.3 (LCMS); ¹H NMR (400 MHZ, DMSO-d₆) δ 9.09 (s, 1H), 8.94 (d, J=7.6 Hz, 1H), 8.82 (dd, J=1.5, 4.1 Hz, 1H), 7.49-7.37 (m, 2H), 7.31 (d, J=2.5 Hz, 1H), 7.02 (d, J=8.5 Hz, 1H), 6.83 (dd, J=2.8, 8.4 Hz, 1H), 6.61 (d, J=2.6 Hz, 1H), 4.43 (br s, 1H), 3.92 (s, 3H), 2.88 (br d, J=12.5 Hz, 1H), 2.58 (br dd, J=10.1, 19.3 Hz, 3H), 2.18-1.88 (m, 10H), 1.82 (br d, J=13.0 Hz, 1H), 1.74-1.63 (m, 1H), 1.53-1.38 (m, 2H), 1.37-1.31 (m, 2H), 1.30-1.23 (m, 1H), 1.22-1.14 (m, 2H).

### Example 422: N-(1-(7-Methoxyquinolin-5-yl)cyclopropyl)-2-methyl-5-((2-methyl-1,2,3,4-tetrahydroisoquinolin-3-yl)methoxy)benzamide (Compound 799)

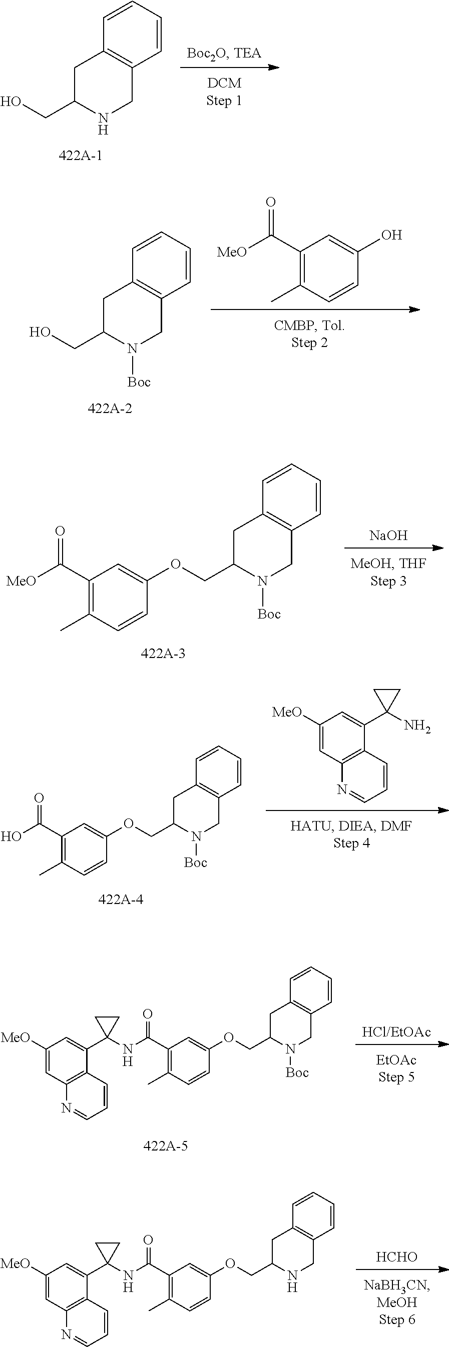

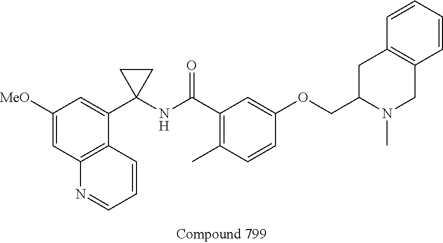 [SMILES: COc1cc(C2(NC(=O)c3cc(OCC4Cc5ccccc5CN4C)ccc3C)CC2)c2cccnc2c1]

### Step 1: tert-Butyl 3-(hydroxymethyl)-3,4-dihydroisoquinoline-2(1H)-carboxylate (422A-2)

To a solution of (1,2,3,4-tetrahydroisoquinolin-3-yl)methanol (200 mg, 1.23 mmol, 1.0 eq) in DCM (10 mL) were added TEA (450 mg, 4.44 mmol, 620 μL, 3.6 eq) and Boc₂O (321 mg, 1.47 mmol, 1.2 eq). The mixture was stirred at 20° C. for 1 h. LCMS indicated that the starting material was completely consumed. The reaction mixture was poured into H₂O (4.0 mL) and extracted with DCM (3.0 mL×2). The combined organic layers were dried over Na₂SO₄, filtered, and concentrated under vacuum to give a residue which was purified by flash silica gel chromatography using a gradient of EtOAc/petroleum ether from 1/100 to 1/10. tert-Butyl 3-(hydroxymethyl)-3,4-dihydroisoquinoline-2(1H)-carboxylate (320 mg, 1.22 mmol, 99% yield) was obtained as a yellow oil. M−56+H⁺=208.2 (LCMS).

### Step 2: tert-Butyl 3-((3-(methoxycarbonyl)-4-methylphenoxy)methyl)-3,4-dihydroisoquinoline-2(1H)-carboxylate (422A-3)

To a solution of tert-butyl 3-(hydroxymethyl)-3,4-dihydroisoquinoline-2(1H)-carboxylate (300 mg, 1.14 mmol, 1.0 eq) and methyl 5-hydroxy-2-methylbenzoate (189 mg, 1.14 mmol, 1.0 eq) in tolene (15 mL) were added TMAD (589 mg, 3.42 mmol, 3.0 eq) and PPh₃ (896 mg, 3.42 mmol, 3.0 eq). The mixture was stirred at 100° C. for 16 h under a N₂ atmosphere. LCMS indicated that the starting material was completely consumed. The reaction mixture was allowed to cool to room temperature, poured into H₂O (15 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 1/100 to 1/10. tert-Butyl 3-((3-(methoxycarbonyl)-4-methylphenoxy)methyl)-3,4-dihydroisoquinoline-2(1H)-carboxylate (366 mg, 890 μmol, 78% yield) was obtained as a yellow oil. M−56+H⁺=356.2 (LCMS).

### Step 3: 5-((2-(tert-Butoxycarbonyl)-1,2,3,4-tetrahydroisoquinolin-3-yl)methoxy)-2-methylbenzoic acid (422A-4)

To a solution of tert-butyl 3-((3-(methoxycarbonyl)-4-methylphenoxy)methyl)-3,4-dihydroisoquinoline-2(1H)-carboxylate (366 mg, 890 μmol, 1.0 eq) in MeOH (20 mL) and THF (10 mL) was added NaOH (2 M aqueous, 1.8 mL, 4.0 eq). The mixture was stirred at 70° C. for 16 h.

LCMS indicated that the starting material was completely consumed. The reaction mixture was allowed to cool to room temperature, poured into H₂O (30 mL), and washed with MTBE (15 mL×2). The aqueous layer was acidified to pH 6 with HCl (1 M aqueous). The product was extracted with EtOAc (30 mL×3). The combined organic layers were dried over Na₂SO₄, filtered, and concentrated under vacuum to give the crude product 5-((2-(tert-butoxycarbonyl)-1,2,3,4-tetrahydroisoquinolin-3-yl)methoxy)-2-methylbenzoic acid (375 mg) as a yellow oil. M−56+H⁺=342.2 (LCMS).

### Step 4: tert-Butyl 3-((3-((1-(7-methoxyquinolin-5-yl)cyclopropyl)carbamoyl)-4-methylphenoxy)methyl)-3,4-dihydroisoquinoline-2(1H)-carboxylate (422A-5)

To a solution of 1-(7-methoxyquinolin-5-yl)cyclopropan-1-amine (80.9 mg, 177 μmol, 1.0 eq) and 5-((2-(tert-butoxycarbonyl)-1,2,3,4-tetrahydroisoquinolin-3-yl)methoxy)-2-methylbenzoicacid (150 mg, 377 μmol, 1.0 eq) in DMF (8.0 mL) were added DIEA (146 mg, 1.13 mmol, 197 μL, 3.0 eq) and HATU (359 mg, 943 μmol, 2.5 eq). The mixture was stirred at 20° C. for 16 h. LCMS indicated that the starting material was completely consumed. The reaction mixture was poured into H₂O (10 mL) and extracted with EtOAc (8.0 mL×3). The combined organic layers were dried over Na₂SO₄, filtered, and concentrated under vacuum to give a residue which was purified by flash silica gel chromatography using a gradient of EtOAc/petroleum ether from 1/100 to 3/5. tert-Butyl 3-((3-((1-(7-methoxyquinolin-5-yl)cyclopropyl)carbamoyl)-4-methylphenoxy)methyl)-3,4-dihydroisoquinoline-2(1H)-carboxylate (193 mg, 325 μmol, 86% yield) was obtained as a yellow oil. M−56+H⁺=538.4 (LCMS).

### Step 5: N-(1-(7-Methoxyquinolin-5-yl)cyclopropyl)-2-methyl-5-((1,2,3,4-tetrahydroisoquinolin-3-yl)methoxy)benzamide (422A-6)

To a solution of tert-butyl 3-((3-((1-(7-methoxyquinolin-5-yl)cyclopropyl)carbamoyl)-4-methylphenoxy)methyl)-3,4-dihydroisoquinoline-2(1H)-carboxylate (193 mg, 325 μmol, 1.0 eq) in EtOAc (5.0 mL) was added HCl/EtOAc (4 M, 7.4 mL). The mixture was stirred at 20° C. for 1 h. LCMS indicated that the starting material was completely consumed. The reaction mixture was concentrated under vacuum to give the product N-(1-(7-methoxyquinolin-5-yl)cyclopropyl)-2-methyl-5-((1,2,3,4-tetrahydroisoquinolin-3-yl)methoxy)benzamide (160 mg, 302 μmol, 93% yield, HCl salt) as a white solid. M+H⁺=494.3 (LCMS).

### Step 6: N-(1-(7-Methoxyquinolin-5-yl)cyclopropyl)-2-methyl-5-((2-methyl-1,2,3,4-tetrahydroisoquinolin-3-yl)methoxy)benzamide (Compound 799)

To a solution of N-(1-(7-methoxyquinolin-5-yl)cyclopropyl)-2-methyl-5-((1,2,3,4-tetrahydroisoquinolin-3-yl)methoxy)benzamide (150 mg, 283 μmol, 1.0 eq, HCl salt) in MeOH (10 mL) was added TEA (1.0 mL), followed by formaldehyde (45.9 mg, 566 μmol, 42.1 μL, 37% purity in water, 2.0 eq). The resulting mixture was adjusted to pH 6 with a small amount of AcOH, then NaBH₃CN (35.6 mg, 566 μmol, 2.0 eq) was added. The mixture was stirred at 20° C. for 16 h. LCMS indicated that the starting material was completely consumed. The reaction mixture was poured into H₂O (5.0 mL) and extracted with EtOAc (10 mL×3). The combined organic layers were dried over Na₂SO₄, filtered, and concentrated under vacuum to give a residue which was purified by preparative HPLC (column: Phenomenex Luna (80× 30 mm×3 μm); flow rate: 25 mL/min; gradient: 10%-35% B over 8 min; mobile phase A: 0.04% aqueous HCl, mobile phase B: acetonitrile). N-(1-(7-Methoxyquinolin-5-yl)cyclopropyl)-2-methyl-5-((2-methyl-1,2,3,4-tetrahydroisoquinolin-3-yl)methoxy)benzamide (65.1 mg, 120 μmol, 42% yield, HCl salt) was obtained as a yellow solid. M+H⁺=508.4 (LCMS); ¹H NMR (400 MHZ, DMSO-d₆) δ 9.82 (d, J=8.4 Hz, 1H), 9.34 (s, 1H), 9.07 (d, J=5.6 Hz, 1H), 8.03-7.87 (m, 2H), 7.47 (d, J=2.0 Hz, 1H), 7.38-7.24 (m, 4H), 7.13 (d, J=8.4 Hz, 1H), 7.05-6.95 (m, 1H), 6.87 (d, J=2.6 Hz, 1H), 4.65-4.41 (m, 3H), 4.38-4.15 (m, 2H), 4.11 (s, 3H), 3.97 (br dd, J=4.6, 8.9 Hz, 1H), 3.46-3.35 (m, 1H), 3.09-2.87 (m, 3H), 2.03 (s, 3H), 1.62-1.54 (m, 2H), 1.49-1.39 (m, 2H).

### Example 423: (S)—N-(1-(7-Methoxyquinolin-5-yl)cyclopropyl-2,2,3,3-d₄)-2-methyl-5-((1-methylazetidin-2-yl)methoxy)benzamide (Compound 794)

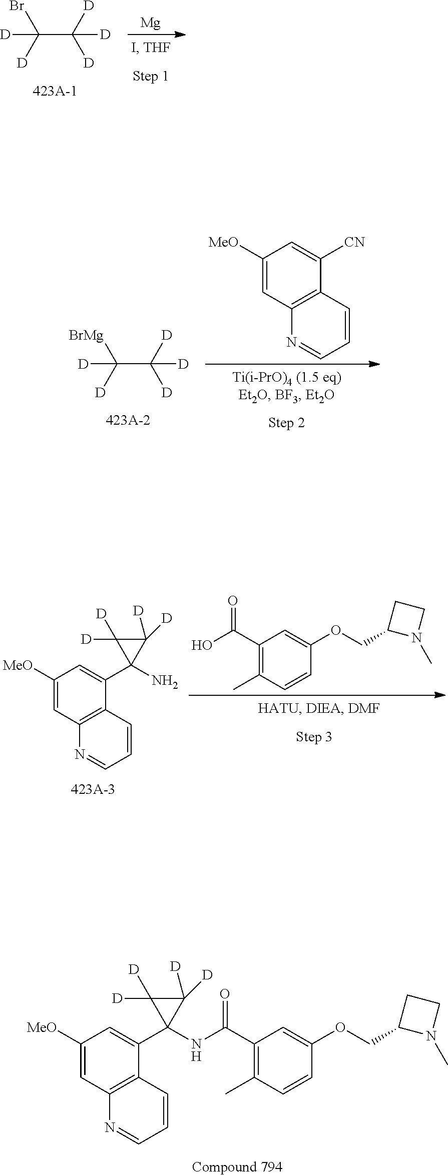

### Step 1: (Ethyl-d₅)magnesium bromide (423A-2)

A 50 mL Schlenk flask was charged with Mg (853 mg, 35.1 mmol, 2.0 eq), degassed, and purged with N₂ three times. Dry THF (18 mL) was added via syringe, and the reaction was cooled to 0° C. I₂ (5 mg, 19.7 μmol) was added, followed by 1-bromoethane-1,1,2,2,2-d₅ (2.00 g, 17.5 mmol, 3.42 mL, 1.0 eq) in portions. The reaction mixture was stirred at 0° C. for 2 h. The reaction mixture turned grey and most of the Mg was consumed. The reaction mixture was used into the next step without any further purification.

### Step 2: 1-(7-Methoxyquinolin-5-yl)cyclopropan-2,2,3,3-d₄-1-amine (423A-3)

A mixture of 7-methoxyquinoline-5-carbonitrile (50.0 mg, 271 μmol, 1.0 eq) in anhydrous THF (5.0 mL) was degassed and purged with N₂ three times. Then the white suspension was cooled to −78° C. To this mixture was added Ti(i-PrO)₄ (116 mg, 407 μmol, 120 μL, 1.5 eq) slowly during a period of 5 min and stirred at −78° C. for 10 min. (Ethyl-d₅) magnesium bromide (1 M in THF, 2.0 mL, 7.4 eq) was added dropwise to maintain the temperature between −78° C. and −75° C. over 10 min under a N₂ atmosphere. The color of the mixture turned brown after the addition was complete. The resulting mixture was stirred at the same temperature for 10 min and then warmed to room temperature (between 15-20° C.) slowly over 1.5 h. The mixture turned black. To the mixture was added BF₃·Et₂O (77.1 mg, 543 μmol, 67.1 μL, 2.0 eq) in portions at the same temperature and no obvious temperature changed was observed. The resulting mixture was stirred at room temperature for another 1 h. LCMS indicated that most of the starting material was consumed, and the desired mass was detected. The reaction mixture was poured into a mixture of HCl (1 M aqueous) (5.0 mL) and MTBE (5.0 mL) and extracted with MTBE (5.0 mL×2). The aqueous layer was basified to pH 8 by using NaOH (2 M aqueous), and a precipitate was formed. The mixture was filtered through a pad of Celite and the slurry was washed with DCM several times. The combined filtrate was extracted with DCM (10 mL×3). The combined organic layers were dried over Na₂SO₄, filtered, and concentrated under vacuum to give a residue which was purified by preparative TLC (DCM/MeOH=10/1, R_{f}=0.4). 1-(7-Methoxyquinolin-5-yl)cyclopropan-2,2,3,3-d₄₋₁-amine (20.0 mg, crude) was obtained as a brown gum. M+H⁺=219.1 (LCMS); ¹H NMR (400 MHZ, CDCl₃) δ 8.85 (dd, J=1.6, 4.3 Hz, 1H), 8.68 (dd, J=0.9, 8.4 Hz, 1H), 7.39-7.33 (m, 2H), 7.24 (d, J=2.5 Hz, 1H), 3.95 (s, 3H).

### Step 3: (S)—N-(1-(7-Methoxyquinolin-5-yl)cyclopropyl-2,2,3,3-d₄)-2-methyl-5-((1-methylazetidin-2-yl)methoxy)benzamide (Compound 794)

To a solution of(S)-2-methyl-5-((1-methylazetidin-2-yl)methoxy)benzoic acid (37.7 mg, 160 μmol, 1.0 eq) and 1-(7-methoxyquinolin-5-yl)cyclopropan-2,2,3,3-d₄-1-amine (35.0 mg, 160 μmol, 1.0 eq) in DMF (1.0 mL) were added DIEA (62.2 mg, 481 μmol, 83.8 μL, 3.0 eq) and HATU (152 mg, 401 μmol, 2.5 eq). The mixture was stirred at 20° C. for 12 h. LCMS indicated that the starting material was completely consumed, and the desired mass was detected. The reaction mixture was filtered to give a filtrate which was purified by preparative HPLC (Phenomenex Luna column (100×30 mm, 5 μm); flow rate: 25 mL/min; gradient: 1%-25% B over 8 min; mobile phase A: 0.1% aqueous TFA, mobile phase B: acetonitrile). (S)—N-(1-(7-Methoxyquinolin-5-yl)cyclopropyl-2,2,3,3-d₄)-2-methyl-5-((1-methylazetidin-2-yl)methoxy)benzamide (6.60 mg, 11.5 μmol, 7% yield, TFA salt) was obtained as a yellow solid. M+H⁺=436.2 (LCMS); ¹H NMR (400 MHZ, DMSO-d₆) δ 10.10-9.90 (m, 1H), 9.21-9.12 (m, 2H), 8.95 (d, J=3.5 Hz, 1H), 7.66-7.55 (m, 2H), 7.39 (d, J=2.4 Hz, 1H), 7.10 (d, J=8.6 Hz, 1H), 6.92 (dd, J=2.8, 8.4 Hz, 1H), 6.77-6.70 (m, 1H), 4.61 (br d, J=4.4 Hz, 1H), 4.23 (d, J=5.3 Hz, 2H), 4.02 (br dd, J=4.9, 9.7 Hz, 1H), 3.96 (s, 3H), 3.87 (br dd, J=6.0, 9.5 Hz, 1H), 2.84 (d, J=4.6 Hz, 3H), 2.41-2.27 (m, 2H), 1.95 (s, 3H).

### Example 424: (S)-4-Hydroxy-N-(1-(7-methoxyquinolin-5-yl)cyclopropyl)-2-methyl-5-((1-methylazetidin-2-yl)methoxy)benzamide (Compound 750)

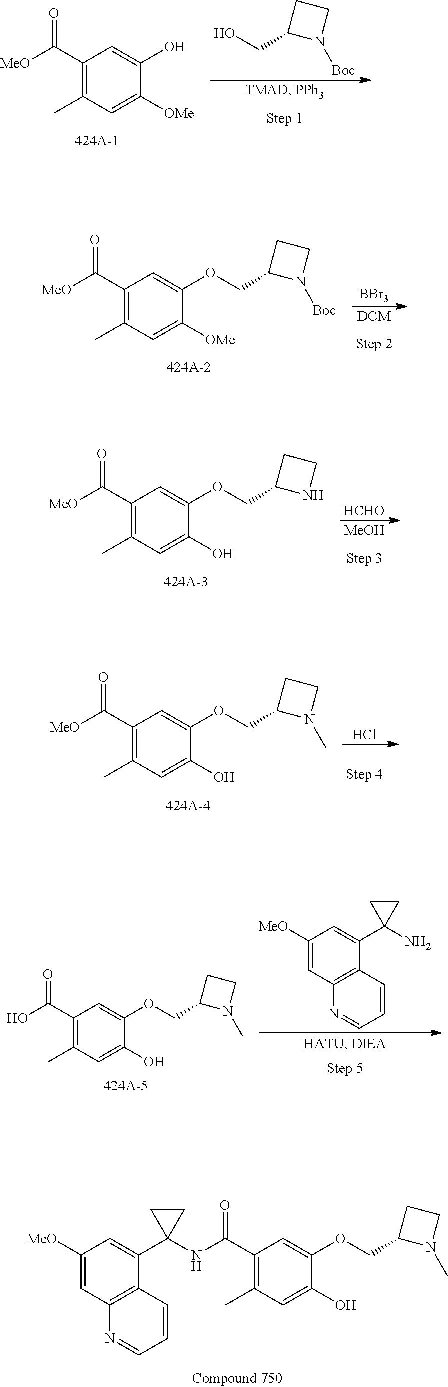

### Step 1: tert-Butyl(S)-2-((2-methoxy-5-(methoxycarbonyl)-4-methylphenoxy)methyl)azetidine-1-carboxylate (424A-2)

To a mixture of methyl 5-hydroxy-4-methoxy-2-methylbenzoate (500 mg, 2.54 mmol, 1.0 eq) and tert-butyl(S)-2-(hydroxymethyl)azetidine-1-carboxylate (578 mg, 2.54 mmol, 1.0 eq) in toluene (12 mL) were added TMAD (1.31 g, 7.64 mmol, 3.0 eq) and PPh₃ (2.00 g, 7.64 mmol, 3.0 eq). The mixture was stirred at 100° C. for 16 h under a N₂ atmosphere. The reaction mixture was allowed to cool to room temperature, poured into H₂O (30 mL) and extracted with EtOAc (20 mL×4). The combined organic layers were dried over Na₂SO₄, filtered, and concentrated under vacuum to give a residue which was purified by flash silica gel chromatography using a gradient of EtOAc/petroleum ether from 1/100 to 1/5. tert-Butyl(S)-2-((2-methoxy-5-(methoxycarbonyl)-4-methylphenoxy)methyl)azetidine-1-carboxylate (900 mg, 2.46 mmol, 97% yield) was obtained as a yellow oil. M−100+H⁺=266.1 (LCMS).

### Step 2: Methyl(S)-5-(azetidin-2-ylmethoxy)-4-hydroxy-2-methylbenzoate (424A-3)

To a solution of tert-butyl(S)-2-((2-methoxy-5-(methoxycarbonyl)-4-methylphenoxy)methyl)azetidine-1-carboxylate (450 mg, 1.23 mmol, 1.0 eq) in DCM (10 mL) was added a solution of BBr₃ (1.54 g, 6.16 mmol, 593 μL, 5.0 eq) in DCM (2.0 mL) dropwise at −78° C. The resulting mixture was stirred at the same temperature for 1 h, then warmed to 20° C. and stirred another 3 h under a N₂ atmosphere. LCMS indicated that the starting material was completely consumed, and the desired mass was detected. The reaction mixture was concentrated under vacuum at 30° C. to give the crude methyl(S)-5-(azetidin-2-ylmethoxy)-4-hydroxy-2-methylbenzoate (800 mg, 2.41 mmol, HBr salt) as a red oil, which was used in the next step without any further purification. M+H⁺=252.2 (LCMS).

### Step 3: Methyl(S)-4-hydroxy-2-methyl-5-((1-methylazetidin-2-yl)methoxy)benzoate (424A-4)

To a solution of methyl(S)-5-(azetidin-2-ylmethoxy)-4-hydroxy-2-methylbenzoate (400 mg, 1.20 mmol, 1.0 eq, HBr salt) in MeOH (10 mL) was added TEA (100 μL), followed by formaldehyde (195 mg, 2.41 mmol, 179 μL, 37% purity in water, 2.0 eq). The resulting mixture was adjusted to pH 6 with a small amount of AcOH. The mixture was stirred at 20° C. for 30 min, then NaBH₃CN (151 mg, 2.41 mmol, 2.0 eq) was added. The resulting reaction mixture was stirred at 20° C. for another 1 h. LCMS indicated that the starting material was completely consumed, and the desired mass was detected. The reaction mixture was poured into H₂O (20 mL) and extracted with DCM (30 mL×5). The combined organic layers were dried over Na₂SO₄, filtered, and concentrated under vacuum to give methyl(S)-4-hydroxy-2-methyl-5-((1-methylazetidin-2-yl)methoxy)benzoate (400 mg) as a yellow oil, which was used in the next step without any further purification. M+H⁺=266.3 (LCMS).

### Step 4: (S)-4-Hydroxy-2-methyl-5-((1-methylazetidin-2-yl)methoxy)benzoic acid (424A-5)

A solution of methyl(S)-4-hydroxy-2-methyl-5-((1-methylazetidin-2-yl)methoxy)benzoate (300 mg, 1.13 mmol, 1.0 eq) in HCl (2 M aqueous, 15 mL) was stirred at 100° C. for 16 h. LCMS indicated that the starting material was completely consumed, and the desired mass was detected. The reaction mixture was allowed to cool to room temperature, treated with H₂O (10 mL) and washed with MTBE (20 mL×2). The aqueous was acidified to pH 6 with NaOH (2 M aqueous). The product was extracted with DCM (20 mL×5) and the combined organic layers were dried over Na₂SO₄, filtered, and concentrated in vacuum to give 2-methyl-5-((1-methylazetidin-2-yl)methoxy)benzoic acid (150 mg) as a white solid, which was used in the next step without any further purification. M+H⁺=252.2 (LCMS).

### Step 5: (S)-4-Hydroxy-N-(1-(7-methoxyquinolin-5-yl)cyclopropyl)-2-methyl-5-((1-methylazetidin-2-yl)methoxy)benzamide (Compound 750)

To a solution of 2-methyl-5-((1-methylazetidin-2-yl)methoxy)benzoic acid (60.0 mg, 239 μmol, 1.0 eq) and 1-(7-methoxyquinolin-5-yl)cyclopropan-1-amine (56.3 mg, 263 μmol, 1.1 eq) in DMF (5.0 mL) were added DIEA (30.9 mg, 239 μmol, 41.6 μL, 1.0 eq) and HATU (27.0 mg, 71.6 μmol, 0.3 eq). The mixture was stirred at 20° C. for 16 h. LCMS indicated that starting material was completely consumed, and the desired mass was detected. The reaction mixture was poured into H₂O (5.0 mL) and extracted with DCM (10 mL×5). The combined organic layers were dried over Na₂SO₄, filtered, and concentrated under vacuum to give a residue which was purified by preparative HPLC (Phenomenex Gemini C18 column (80×30 mm, 3 μm); flow rate: 25 mL/min; gradient: 1%-20% B over 8 min; mobile phase A: 0.04% aqueous HCl, mobile phase B: acetonitrile). (S)-4-Hydroxy-N-(1-(7-methoxyquinolin-5-yl)cyclopropyl)-2-methyl-5-((1-methylazetidin-2-yl)methoxy)benzamide (34.0 mg, 70.3 μmol, 29% yield, HCl salt) was obtained as a brown solid. M+H⁺=448.2 (LCMS); ¹H NMR (400 MHZ, DMSO-d₆) δ 9.55-9.45 (m, 1H), 9.11-8.98 (m, 2H), 7.87-7.77 (m, 1H), 7.70-7.62 (m, 1H), 7.50-7.42 (m, 1H), 6.87-6.80 (m, 1H), 6.64-6.59 (m, 1H), 4.74-4.51 (m, 1H), 4.31-4.18 (m, 2H), 4.07-3.80 (m, 5H), 2.93-2.81 (m, 3H), 2.41-2.32 (m, 2H), 2.01-1.95 (m, 3H), 1.41-1.36 (m, 2H), 1.30-1.24 (m, 2H).

### Example 425: (S)—N-(1-(7-Methoxyquinolin-5-yl)cyclopropyl)-2-methyl-4-(methylamino)-5-((1-methylazetidin-2-yl)methoxy)benzamide (Compound 737)

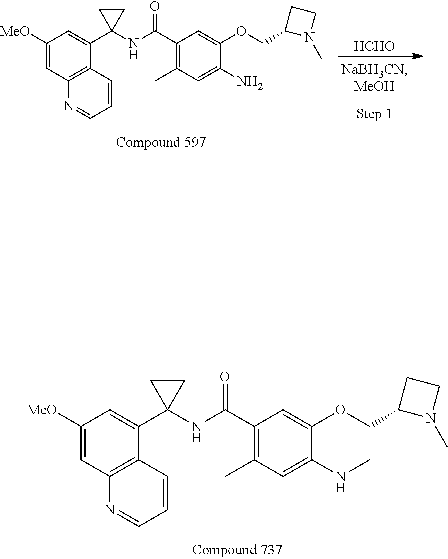

### Step 1: (S)—N-(1-(7-Methoxyquinolin-5-yl)cyclopropyl)-2-methyl-4-(methylamino)-5-((1-methylazetidin-2-yl)methoxy)benzamide (Compound 737)

To a solution of(S)-4-amino-N-(1-(7-methoxyquinolin-5-yl)cyclopropyl)-2-methyl-5-((1-methylazetidin-2-yl)methoxy)benzamide (46.0 mg, 103 μmol, 1.0 eq) in MeOH (5.0 mL) was added TEA (1.0 mL), followed by formaldehyde (8.36 mg, 103 μmol, 7.68 μL, 37% purity in water, 2.0 eq). The resulting mixture was adjusted to pH 6 with a small amount of AcOH. Then NaBH₃CN (9.72 mg, 155 μmol, 2.0 eq) was added. The mixture was stirred at 20° C. for 16 h. LCMS indicated that the starting material was completely consumed. The reaction mixture was poured into H₂O (5.0 mL) and extracted with EtOAc (2.0 mL×3). The combined organic layers were dried over Na₂SO₄, filtered, and concentrated under vacuum to give a residue which was purified by preparative HPLC (column: Phenomenex Luna (80×30 mm, 3 μm); flow rate: 25 mL/min; gradient: 1%-20% B over 8 min; mobile phase A: 0.04% aqueous HCl, mobile phase B: acetonitrile). (S)—N-(1-(7-Methoxyquinolin-5-yl)cyclopropyl)-2-methyl-4-(methylamino)-5-((1-methylazetidin-2-yl)methoxy)benzamide (14.5 mg, 29.2 μmol, 28% yield, HCl salt) was obtained as a yellow solid. M+H⁺=461.3 (LCMS); ¹H NMR (400 MHZ, DMSO-d₆) δ 10.59 (br s, 1H), 9.71 (br dd, J=2.0, 7.8 Hz, 1H), 9.14 (br d, J=5.6 Hz, 1H), 8.95 (s, 1H), 7.91 (br dd, J=4.1, 7.6 Hz, 1H), 7.71 (d, J=2.4 Hz, 1H), 7.53 (d, J=1.8 Hz, 1H), 6.80 (s, 1H), 6.31 (br s, 1H), 4.73-4.51 (m, 1H), 4.32 (dd, J=5.9, 12.1 Hz, 1H), 4.18 (dd, J=2.6, 11.9 Hz, 1H), 4.04 (br dd, J=2.3, 5.9 Hz, 1H), 4.01 (s, 3H), 3.87-3.84 (m, 1H), 2.83 (s, 3H), 2.73-2.69 (m, 3H), 2.42-2.34 (m, 2H), 2.08 (s, 3H), 1.40 (br d, J=0.9 Hz, 2H), 1.31-1.24 (m, 2H).

### Example 426: (S)—N-(1-(7-Methoxy-2-methylquinolin-5-yl)cyclopropyl)-2-methyl-5-((1-methylazetidin-2-yl)methoxy)benzamide (Compound 625)

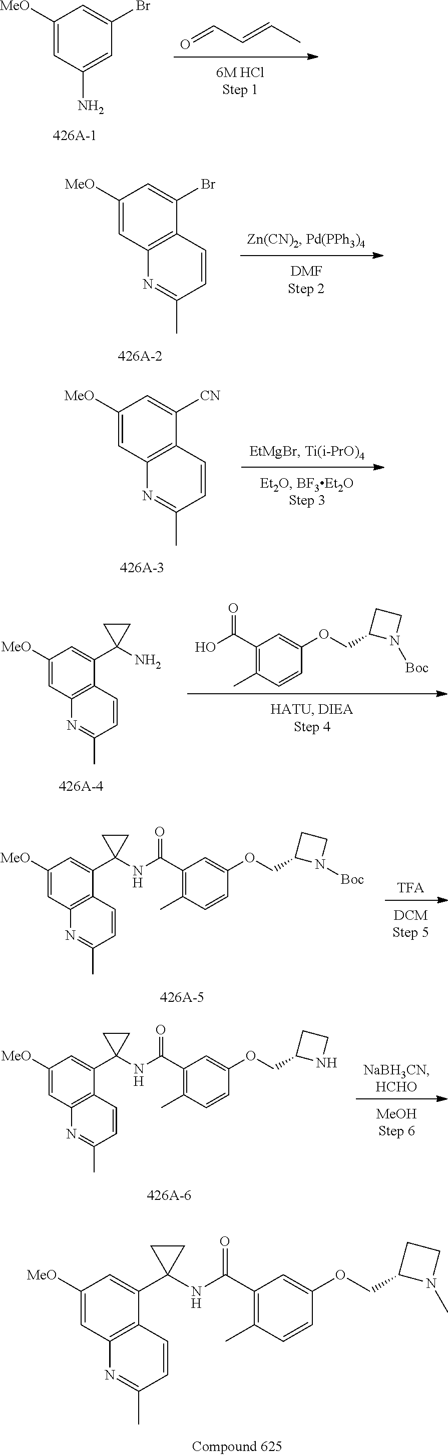

### Step 1: 5-Bromo-7-methoxy-2-methylquinoline (426A-2)

A mixture of 3-bromo-5-methoxyaniline (2.50 g, 12.4 mmol, 1.0 eq) and HCl (6 M aqueous, 10 mL) was heated to 105° C., then (E)-but-2-enal (1.73 g, 24.8 mmol, 2.1 mL, 2.0 eq) was added slowly. The resulting mixture was stirred at 105° C. for 1 h. LCMS indicated that the starting material was completely consumed, and the desired mass was detected. The reaction mixture was allowed to cool to room temperature, poured into ice-cold water (20 mL), treated with NH₃H₂O to adjust pH 8, and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 1/100 to 1/10. 5-Bromo-7-methoxy-2-methylquinoline (750 mg, 2.98 mmol, 24% yield) was obtained as a yellow solid. M+H⁺=252.1 (LCMS); ¹H NMR (400 MHZ, DMSO-d₆) δ 8.25 (d, J=8.5 Hz, 1H), 7.57 (d, J=2.5 Hz, 1H), 7.45-7.35 (m, 2H), 3.91 (s, 3H), 2.65 (s, 3H).

### Step 2: 7-Methoxy-2-methylquinoline-5-carbonitrile (426A-3)

To a solution of 5-bromo-7-methoxy-2-methyl-quinoline (1.75 g, 6.94 mmol, 1.0 eq) in DMF (25 mL) were added Zn(CN)₂ (1.63 g, 13.9 mmol, δ81 μL, 2.0 eq) and Pd(PPh₃)₄ (802 mg, 694 μmol, 0.1 eq). The mixture was degassed and purged with N₂ three times. The resulting mixture was stirred at 100° C. for 12 h under a N₂ atmosphere. LCMS indicated that the starting material was completely consumed, and the desired mass was detected. The reaction mixture was allowed to cool to room temperature, poured into ice-cold water (30 mL), and extracted with EtOAc (25 mL×3). The combined organic layers were dried over Na₂SO₄, filtered, and concentrated under vacuum to give a residue which was purified by flash silica gel chromatography using a gradient of EtOAc/petroleum ether from 1/100 to 3/5. 7-Methoxy-2-methylquinoline-5-carbonitrile (980 mg, 4.95 mmol, 73% yield) was obtained as a yellow solid. M+H⁺=199.3 (LCMS); ¹H NMR (400 MHZ, DMSO-d₆) δ 8.27 (d, J=8.5 Hz, 1H), 7.91 (d, J=2.5 Hz, 1H), 7.69 (d, J=2.4 Hz, 1H), 7.51 (d, J=8.5 Hz, 1H), 3.96 (s, 3H), 2.68 (s, 3H).

### Step 3: 1-(7-Methoxy-2-methylquinolin-5-yl)cyclopropanamine (426A-4)

A solution of 7-methoxy-2-methylquinoline-5-carbonitrile (1.30 g, 6.56 mmol, 1.0 eq) in Et₂O (160 mL) was degassed and purged with N₂ three times and cooled to −78° C. Ti(i-PrO)₄ (2.80 g, 9.84 mmol, 2.90 mL, 1.5 eq) was added slowly and the mixture was stirred for 5 min. EtMgBr (3.0 M in Et₂O, 4.81 mL, 2.2 eq) was added dropwise at −78° C. to maintain the temperature at −78° C. under a N₂ atmosphere. The mixture was stirred at same temperature for 10 min then warmed to 20° C. over 1 h. The mixture turned to black. To the mixture was added BF₃·Et₂O (1.86 g, 13.1 mmol, 1.62 mL, 2.0 eq) in portions at the same temperature with no obvious temperature change. The resulting mixture was stirred at room temperature for another 1 h. LCMS showed some the starting material was completely consumed, and the desired mass was detected. The reaction mixture was poured into a mixture of HCl (1 M aqueous, 100 mL) and MTBE (100 mL) and extracted with MTBE (80 mL×2). The aqueous layer was basified to pH 8 using NaOH (2 M aqueous) and a precipitate was formed. The mixture was filtered through a pad of Celite and the slurry was washed with DCM several times. The combined filtrate was extracted with DCM (50 mL×8). The combined organic layers were dried over Na₂SO₄, filtered, and concentrated under vacuum to give a residue which was purified by flash silica gel chromatography using a gradient of EtOAc/petroleum ether from 0/1 to 1/1. 1-(7-Methoxy-2-methylquinolin-5-yl)cyclopropanamine (700 mg, 3.07 mmol, 47% yield) was obtained as a yellow oil. M+H⁺=229.2 (LCMS); ¹H NMR (400 MHZ, CDCl₃) δ 8.56 (d, J=8.4 Hz, 1H), 7.30 (d, J=2.5 Hz, 1H), 7.24 (d, J=8.5 Hz, 1H), 7.17 (d, J=2.5 Hz, 1H), 3.94 (s, 3H), 2.73 (s, 3H), 1.21-1.13 (m, 2H), 0.99 (d, J=2.1 Hz, 2H).

### Step 4: (S)-tert-Butyl 2-((3-((1-(7-methoxy-2-methylquinolin-5-yl)cyclopropyl)carbamoyl)-4-methylphenoxy)methyl)azetidine-1-carboxylate (426A-5)

To a solution of(S)-5-((1-(tert-butoxycarbonyl)azetidin-2-yl)methoxy)-2-methylbenzoic acid (211 mg, 657 μmol, 1.0 eq) in DMF (5.0 mL) was added 1-(7-methoxy-2-methylquinolin-5-yl)cyclopropanamine (150 mg, 657 μmol, 1.0 eq), followed by DIEA (255 mg, 1.97 mmol, 343 μL, 3.0 eq) and HATU (375 mg, 986 μmol, 1.5 eq). The resulting mixture was stirred at 25° C. for 8 h. LCMS indicated that the starting material was completely consumed, and the desired mass was detected. The mixture was treated with ice-water (15 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/0. (S)-tert-Butyl 2-((3-((1-(7-methoxy-2-methylquinolin-5-yl)cyclopropyl)carbamoyl)-4-methylphenoxy)methyl)azetidine-1-carboxylate (220 mg, 414 μmol, 63% yield) was obtained as a yellow oil. M+H⁺=532.3 (LCMS).

### Step 5: (S)-5-(Azetidin-2-ylmethoxy)-N-(1-(7-methoxy-2-methylquinolin-5-yl)cyclopropyl)-2-methylbenzamide (426A-6)

To a solution of(S)-tert-butyl 2-((3-((1-(7-methoxy-2-methylquinolin-5-yl)cyclopropyl) carbamoyl)-4-methylphenoxy)methyl)azetidine-1-carboxylate (370 mg, 696 μmol, 1.0 eq) in DCM (12 mL) was added TFA (4.0 mL). The mixture was stirred at 25° C. for 30 min. LCMS indicated that the starting material was completely consumed, and the desired mass was detected. The mixture was concentrated under vacuum at 20° C. to give the crude(S)-5-(azetidin-2-ylmethoxy)-N-(1-(7-methoxy-2-methylquinolin-5-yl)cyclopropyl)-2-methylbenzamide (370 mg, crude, TFA salt) as a yellow oil. M+H⁺=432.2 (LCMS).

### Step 6: (S)—N-(1-(7-Methoxy-2-methylquinolin-5-yl)cyclopropyl)-2-methyl-5-((1-methylazetidin-2-yl)methoxy)benzamide (Compound 625)

To a solution of(S)-5-(azetidin-2-ylmethoxy)-N-(1-(7-methoxy-2-methylquinolin-5-yl)cyclopropyl)-2-methylbenzamide (220 mg, 403 μmol, 1.0 eq, TFA) in MeOH (3.0 mL) was added TEA (50 μL), followed by HCHO (49.1 mg, 605 μmol, 45 μL, 37% purity, 1.5 eq). The resulting mixture was adjusted to pH 6 with a small amount of AcOH. The mixture was stirred at 20° C. for 30 min, then NaBH₃CN (30.4 mg, 484 μmol, 1.2 eq) was added. The resulting mixture was stirred at 25° C. for 4 h. LCMS indicated that the starting material was completely consumed, and the desired mass was detected. The mixture was treated with NaHCO₃ aqueous (1.0 mL) and concentrated under vacuum to give a residue. The residue was purified by preparative HPLC (Waters Xbridge BEH C18 column (100×30 mm, 10 μm); flow rate: 60 mL/min; gradient: 25%-65% B over 8 min; mobile phase A: 10 mM aqueous NH₄HCO₃, mobile phase B: acetonitrile) to give a white solid. The solid was diluted with a mixture of acetonitrile (1.0 mL), H₂O (4.0 mL), then 146 μL HCl (1 M aqueous) was added slowly. The resulting mixture was lyophilized to give(S)—N-(1-(7-methoxy-2-methylquinolin-5-yl)cyclopropyl)-2-methyl-5-((1-methylazetidin-2-yl)methoy)benzamide (70.1 mg, 145 μmol, 31% yield, HCl salt) as a white solid. M+H⁺=446.4 (LCMS); ¹H NMR (400 MHZ, DMSO-d₆) δ 10.82-10.52 (m, 1H), 9.42-9.02 (m, 2H), 7.57-7.39 (m, 2H), 7.16-7.03 (m, 1H), 6.97-6.89 (m, 1H), 6.80-6.62 (m, 1H), 4.69-4.52 (m, 1H), 4.45-4.28 (m, 1H), 4.22 (dd, J=3.2, 11.2 Hz, 1H), 4.06-3.90 (m, 4H), 3.85 (br dd, J=6.2, 9.5 Hz, 1H), 2.83-2.66 (m, 6H), 2.47-2.19 (m, 3H), 1.96 (s, 3H), 1.36 (br s, 2H), 1.23 (br s, 2H).

### Example 427: (S)-2-Fluoro-N-(1-(7-methoxy-2-methylquinolin-5-yl)cyclopropyl)-5-((1-methylazetidin-2-yl)methoxy)benzamide (Compound 791)

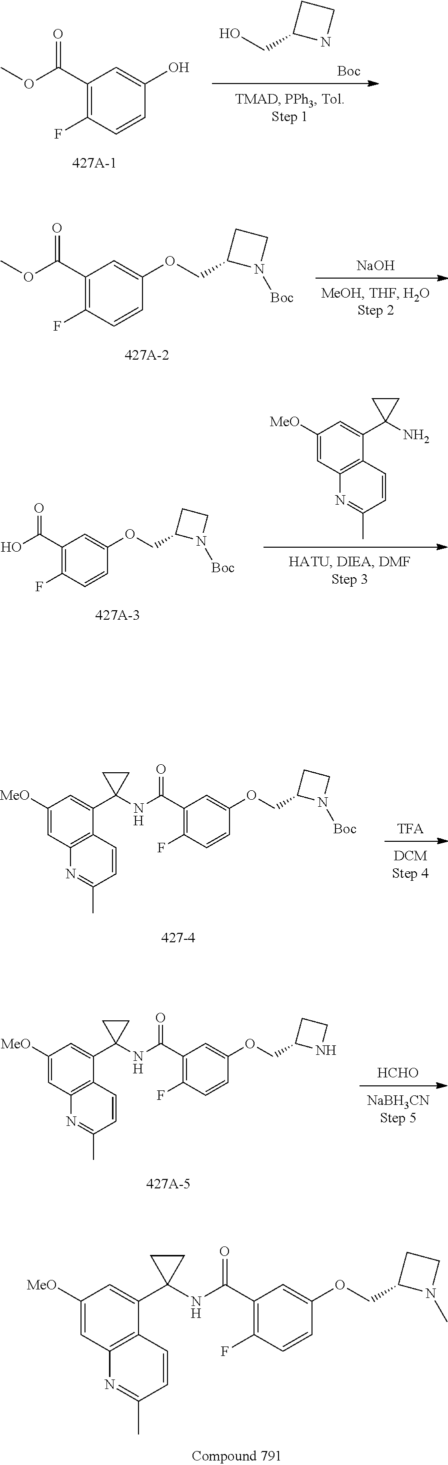

### Step 1: (S)-tert-Butyl 2-((4-fluoro-3-(methoxycarbonyl)phenoxy)methyl)azetidine-1-carboxylate (427A-2)

To a solution of methyl 2-fluoro-5-hydroxybenzoate (500 mg, 2.94 mmol, 1.0 eq) and tert-butyl(S)-2-(hydroxyl methyl)azetidine-1-carboxylate (550 mg, 2.94 mmol, 1.0 eq) in toluene (10 mL) were added TMAD (759 mg, 4.41 mmol, 1.5 eq) and PPh₃ (1.54 g, 5.88 mmol, 2.0 eq). The mixture was stirred at 110° C. for 12 h under a N₂ atmosphere. LCMS indicated that the starting material was completely consumed, and the desired mass was detected. The reaction mixture was allowed to cool to room temperature and concentrated under vacuum to give a residue which was purified by preparative TLC (EtOAc/petroleum ether=1/3, R_{f}=0.47). (S)-tert-Butyl 2-((4-fluoro-3-(methoxycarbonyl)phenoxy)methyl)azetidine-1-carboxylate (950 mg, 2.80 mmol, 95% yield) was obtained as a yellow oil. M+H⁺=340.2 (LCMS).

### Step 2: (S)-5-((1-(tert-Butoxycarbonyl)azetidin-2-yl)methoxy)-2-fluorobenzoic acid (427A-3)

To a solution of tert-butyl(S)-2-((4-fluoro-3-(methoxycarbonyl)phenoxy)methyl)azetidine-1-carboxylate (950 mg, 2.80 mmol, 1.0 eq) in a mixture of MeOH (3.0 mL) and THF (9.0 mL) was added NaOH (2 M aqueous, 5.60 mL, 4.0 eq). The mixture was stirred at 70° C. for 12 h. LCMS indicated that the starting material was completely consumed, and the desired mass was detected. The reaction mixture was allowed to cool to room temperature and concentrated under vacuum to give a residue. The residue was diluted with HCl (2 M aqueous) (10 mL) and extracted with EtOAc (10 mL×3). The combined organic layers were dried over Na₂SO₄, filtered, and concentrated under vacuum to give(S)-5-((1-(tert-butoxycarbonyl)azetidin-2-yl)methoxy)-2-fluorobenzoic acid (650 mg) as a yellow solid. M+H⁺=326.1 (LCMS).

### Step 3: (S)-tert-Butyl 2-((4-fluoro-3-((1-(7-methoxy-2-methylquinolin-5-yl)cyclopropyl)carbamoyl)phenoxy)methyl)azetidine-1-carboxylate (427A-4)

To a solution of(S)-5-((1-(tert-butoxycarbonyl)azetidin-2-yl)methoxy)-2-fluorobenzoic acid (50.0 mg, 154 μmol, 1.0 eq) and 1-(7-methoxy-2-methylquinolin-5-yl)cyclopropanamine (35.1 mg, 154 μmol, 1.0 eq) in DMF (1.0 mL) were added HATU (87.7 mg, 230 μmol, 1.5 eq) and DIEA (59.6 mg, 461 μmol, 80.3 μL, 3.0 eq). The mixture was stirred at 20° C. for 1 h. LCMS indicated that the starting material was completely consumed, and the desired mass was detected. The reaction mixture was diluted with H₂O (2.0 mL) and extracted with EtOAc (2.0 mL×3). The combined organic layers were washed with brine (2.0 mL×3), and the combined organic layers were dried over Na₂SO₄, filtered, and concentrated under vacuum to give a residue which was purified by flash silica gel chromatography using a gradient of EtOAc/petroleum ether from 0/1 to 1/1. (S)-tert-Butyl 2-((4-fluoro-3-((1-(7-methoxy-2-methylquinolin-5-yl)cyclopropyl)carbamoyl)phenoxy)methyl)azetidine-1-carboxylate (25.0 mg, 46.7 μmol, 30% yield) was obtained as a yellow solid. M+H⁺=536.4 (LCMS).

### Step 4: (S)-5-(Azetidin-2-ylmethoxy)-2-fluoro-N-(1-(7-methoxy-2-methylquinolin-5-yl)cyclopropyl)benzamide (427A-5)

To a solution of tert-butyl(25)-2-[4-fluoro-3-[1-(7-methoxy-2-methyl-5-quinolyl)cyclopropyl]carbamoyl]phenoxy]methyl]azetidine-1-carboxylate (30.0 mg, 56.0 μmol, 1.0 eq) in DCM (1.0 mL) was added TFA (63.9 mg, 560 μmol, 41.5 μL, 10 eq). The mixture was stirred at 20° C. for 1 h. LCMS indicated that the starting material was completely consumed. The reaction mixture was concentrated under vacuum to give(S)-5-(azetidin-2-ylmethoxy)-2-fluoro-N-(1-(7-methoxy-2-methylquinolin-5-yl)cyclopropyl)benzamide (30.0 mg, TFA salt) as a yellow oil M+H⁺=436.2 (LCMS).

### Step 5: 2-Fluoro-N-[1-(7-methoxy-2-methyl-5-quinolyl)cyclopropyl]-5-[[(2S)-1-methylazetidin-2-yl]methoxy]benzamide (Compound 791)

To a solution of(S)-5-(azetidin-2-ylmethoxy)-2-fluoro-N-(1-(7-methoxy-2-methylquinolin-5-yl)cyclopropyl)benzamide (30.0 mg, 54.6 μmol, 1.0 eq, TFA salt) and HCHO (8.86 mg, 109 μmol, 8.13 μL, 37% purity in H₂O, 2.0 eq) in MeOH (1.0 mL) was added TEA (5.52 mg, 54.6 μmol, 7.60 μL, 1.0 eq) to adjust the pH to 8, then AcOH (3.28 mg, 54.6 μmol, 3.12 μL, 1.0 eq) was added to adjust the pH to 5. The mixture was stirred at 20° C. for 30 min, then NaBH₃CN (10.3 mg, 164 μmol, 3.0 eq) was added, and the resulting mixture was stirred at 20° C. for 12 h. LCMS indicated that the starting material was completely consumed, and the desired mass was detected. The mixture was concentrated under vacuum at 30° C. to give a residue which was purified by preparative HPLC (Phenomenex Gemini C18 column (80×30 mm, 3 μm); flow rate: 25 mL/min; gradient: 5%-25% B over 8 min; mobile phase A: 0.04% aqueous HCl, mobile phase B: acetonitrile). 2-Fluoro-N-[1-(7-methoxy-2-methyl-5-quinolyl)cyclopropyl]-5-[(2S)-1-methylazetidin-2-yl]methoxy]benzamide (7.60 mg, 15.5 μmol, 28% yield, HCl salt) was obtained as a pale yellow gum. M+H⁺=450.4 (LCMS); ¹H NMR (400 MHZ, DMSO-d₆) δ 11.10 (br s, 1H), 9.60-9.41 (m, 2H), 7.84 (br d, J=8.6 Hz, 1H), 7.76-7.61 (m, 2H), 7.21-7.15 (m, 1H), 7.13-7.06 (m, 1H), 7.06-7.01 (m, 1H), 4.70-4.59 (m, 1H), 4.50-4.40 (m, 1H), 4.25 (br dd, J=2.6, 11.0 Hz, 1H), 3.99 (s, 4H), 3.88-3.80 (m, 1H), 2.93 (s, 3H), 2.80 (br d, J=4.8 Hz, 3H), 2.38-2.26 (m, 2H), 1.42 (br s, 2H), 1.30 (br s, 2H).

### Example 428: (S)—N-(1-(7-Methoxy-2-(trifluoromethyl)quinolin-5-yl)cyclopropyl)-2-methyl-5-((1-methylazetidin-2-yl)methoxy)benzamide (Compound 817)

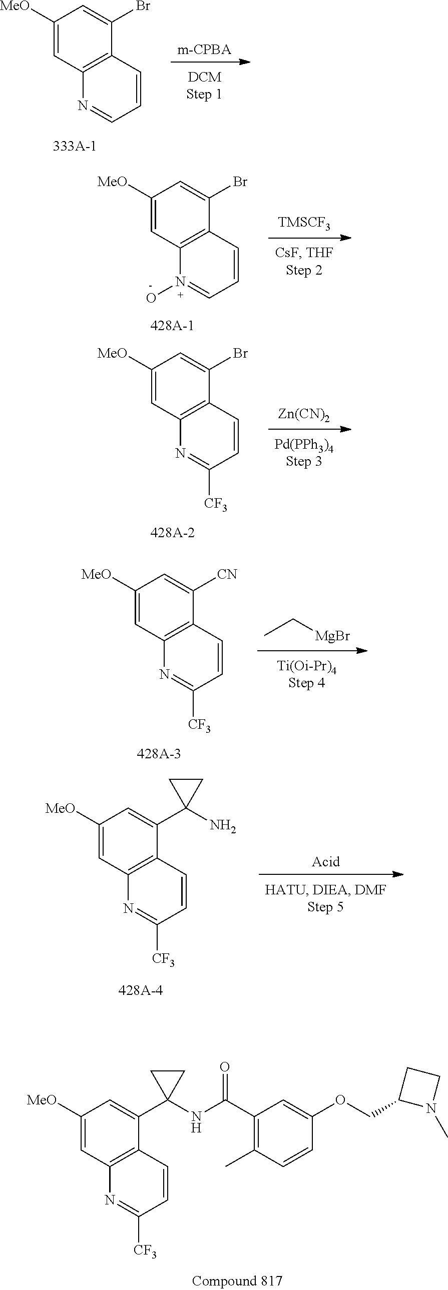

### Step 1: 5-Bromo-7-methoxyquinoline 1-oxide (428A-1)

To a solution of 5-bromo-7-methoxyquinoline (600 mg, 2.52 mmol, 1.0 eq) in DCM (10 mL) was added m-CPBA (665 mg, 3.28 mmol, 1.3 eq). The mixture was stirred at 20° C. for 12 h. LCMS indicated that the starting material was completely consumed. The mixture was treated with HCl (2 M aqueous, 10 mL) and extracted with DCM (10 mL×3). The combined organic layers were dried over Na₂SO₄, filtered, and concentrated under vacuum to give 5-bromo-7-methoxyquinoline 1-oxide (650 mg, crude) as a yellow solid. M+H⁺=254.1/256.1 (LCMS).

### Step 2: 5-Bromo-7-methoxy-2-(trifluoromethyl)quinoline (428A-2)

To a solution of 5-bromo-7-methoxyquinoline 1-oxide (650 mg, 2.56 mmol, 1.0 eq) in THF (10 mL) were added CsF (388 mg, 2.56 mmol, 1.0 eq) and TMSCF₃ (1.46 g, 10.2 mmol, 4.0 eq) at 0° C. The mixture was stirred at 20° C. for 12 h. LCMS indicated that the starting material was completely consumed. The mixture was treated with water (10 mL) and extracted with EtOAc (10 mL×3). The combined organic layers were dried over Na₂SO₄, filtered, and concentrated under vacuum to give a residue which was purified by flash silica gel chromatography using a gradient of EtOAc/petroleum ether from 0/1 to 1/20. 5-Bromo-7-methoxy-2-(trifluoromethyl)quinoline (360 mg, 1.18 mmol, 45% yield) was obtained as a yellow solid. M+H⁺=306.1/308.1 (LCMS).

### Step 3: 7-Methoxy-2-(trifluoromethyl)quinoline-5-carbonitrile (428A-3)

To a solution of 5-bromo-7-methoxy-2-(trifluoromethyl)quinoline (180 mg, 588 μmol, 1.0 eq) in DMF (3.0 mL) were added Zn(CN)₂ (250 mg, 2.13 mmol, 3.6 eq) and Pd(PPh₃)₄ (67.9 mg, 58.8 μmol, 0.10 eq). The resulting mixture was stirred at 80° C. for 12 h under a N₂ atmosphere. LCMS indicated that the starting material was completely consumed. The mixture was treated with water (5.0 mL) and extracted with EtOAc (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/20. 7-Methoxy-2-(trifluoromethyl)quinoline-5-carbonitrile (130 mg, 515 μmol, 87% yield) was obtained as a yellow solid. M+H⁺=253.1 (LCMS).

### Step 4: 1-(7-Methoxy-2-(trifluoromethyl)quinoline-5-yl)cyclopropan-1-amine (428A-4)

A solution of 7-methoxy-2-(trifluoromethyl)quinoline-5-carbonitrile (150 mg, 594 μmol, 1.0 eq) in anhydrous Et₂O (50 mL) was degassed and purged with N₂ three times, The mixture was stirred with a mechanical stirrer at −78° C. To this mixture was added Ti(i-PrO)₄ (185 mg, 654 μmol, 193 μL, 1.1 eq) slowly, and then EtMgBr (3 M in Et₂O, 436 μL, 2.2 eq) was added dropwise to maintain the temperature between −78° C. and −75° C. over 1 h under a N₂ atmosphere. After the addition was complete, the resulting mixture was stirred at the same temperature for 10 min and warmed to 20° C. over 1 h. BF₃·Et₂O (84.0 mg, 594 μmol, 73.4 μL, 1.0 eq) was added slowly with no obvious temperature change. The mixture was stirred at 20° C. for another 1 h. LCMS indicated that the starting material was completely consumed. The reaction mixture was added into a mixture of HCl (1 M aqueous, 5.0 mL) and MTBE (10 mL) and extracted with MTBE (10 mL×2). The aqueous layer was basified to pH 8 by using NaOH (2 M aqueous) and extracted with DCM (5.0 mL×3). The combined organic layers were dried over Na₂SO₄, filtered, and concentrated under vacuum to give a residue which was purified by flash silica gel chromatography using a gradient of EtOAc/petroleum ether from 0/1 to 1/3 to give 1-(7-methoxy-2-(trifluoromethyl)quinolin-5-yl)cyclopropan-1-amine (30.0 mg, 106 μmol, 87% yield) as a yellow oil. M+H⁺=283.1 (LCMS).

### Step 5: (S)—N-(1-(7-Methoxy-2-(trifluoromethyl)quinolin-5-yl)cyclopropyl)-2-methyl-5-((1-methylazetidin-2-yl)methoxy)benzamide (Compound 817)

To a solution of 1-(7-methoxy-2-(trifluoromethyl)quinolin-5-yl)cyclopropan-1-amine (20.0 mg, 70.8 μmol, 1.0 eq) in DMF (2.0 mL) were added(S)-2-methyl-5-((1-methylazetidin-2-yl)methoxy)benzoic acid (16.6 mg, 70.8 μmol, 1.0 eq), HATU (40.4 mg, 106 μmol, 1.5 eq), and DIEA (27.4 mg, 212 μmol, 37.0 μL, 3.0 eq). The resulting mixture was stirred at 20° C. for 2 h. LCMS indicated that the starting material was completely consumed. The mixture was concentrated under vacuum to give a residue which was purified by preparative HPLC (Phenomenex Gemini C18 column (80×30 mm, 3 μm); flow rate: 60 mL/min; gradient: 25%-55% B over 8 min; mobile phase A: 0.04% aqueous HCl, mobile phase B: acetonitrile) to give(S)—N-(1-(7-methoxy-2-(trifluoromethyl)quinolin-5-yl)cyclopropyl)-2-methyl-5-((1-methylazetidin-2-yl)methoxy)benzamide (4.90 mg, 9.10 μmol, 12% yield) as a white solid. M+H⁺=500.4 (LCMS); ¹H NMR (400 MHZ, DMSO-d₆) δ 10.01-9.81 (m, 1H), 9.27-9.18 (m, 2H), 7.88 (d, J=8.7 Hz, 1H), 7.63 (d, J=2.4 Hz, 1H), 7.49 (d, J=2.3 Hz, 1H), 7.10 (d, J=8.4 Hz, 1H), 6.92 (dd, J=2.8, 8.4 Hz, 1H), 6.71 (d, J=2.6 Hz, 1H), 4.66-4.54 (m, 1H), 4.22 (d, J=5.3 Hz, 2H), 3.97 (s, 3H), 3.91-3.78 (m, 2H), 2.83 (d, J=5.0 Hz, 2H), 2.43-2.27 (m, 3H), 1.95 (s, 3H), 1.36 (br s, 2H), 1.28-1.23 (m, 2H).

### Example 429: (S)—N-(1-(7-Hydroxy-2-methylquinolin-5-yl)cyclopropyl)-2-methyl-5-((1-methylazetidin-2-yl)methoxy)benzamide (Compound 809)

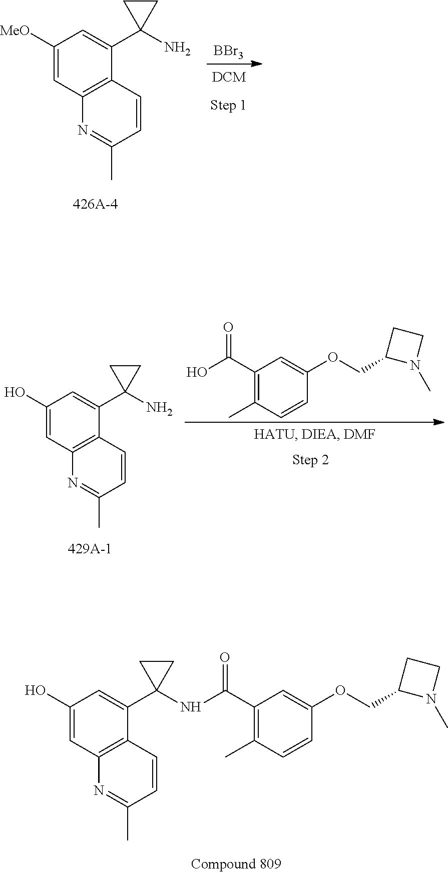

### Step 1: 5-(1-Aminocyclopropyl)-2-methylquinolin-7-ol (429A-1)

To a solution of 1-(7-methoxy-2-methylquinolin-5-yl)cyclopropan-1-amine (1.40 g, 6.13 mmol, 1.0 eq) in DCM (60 mL) was added a solution of BBr₃ (23.0 g, 92.0 mmol, 8.86 mL, 15 eq) in DCM (10 mL) dropwise at −78° C. under a N₂ atmosphere. The resulting mixture was stirred at the same temperature for 2 h, then warmed to 20° C. and stirred another 12 h. LCMS indicated that the starting material was completely consumed, and the desired mass was detected. The reaction mixture was concentrated under vacuum to give a residue, which was diluted with MeOH (20 mL) at 0° C. and treated with NH₃H₂O to adjust the pH 8. The mixture was concentrated under vacuum to give a residue which was purified by flash silica gel chromatography using a gradient of EtOAc/petroleum ether from 0/1 to 1/1, followed by DCM/MeOH from 100/1 to 10/1. 5-(1-Aminocyclopropyl)-2-methylquinolin-7-ol (1.40 g, 6.53 mmol, crude) was obtained as a yellow solid. M+H⁺=215.1 (LCMS).

### Step 2: (S)—N-(1-(7-Hydroxy-2-methylquinolin-5-yl)cyclopropyl)-2-methyl-5-((1-methylazetidin-2-yl)methoxy)benzamide (Compound 809)

To a solution of(S)-2-methyl-5-((1-methylazetidin-2-yl)methoxy)benzoic acid (150 mg, 638 μmol, 1.0 eq) and 5-(1-aminocyclopropyl)-2-methylquinolin-7-ol (137 mg, 638 μmol, 1.0 eq) in DMF (10 mL) were added HATU (170 mg, 446 μmol, 0.7 eq) and DIEA (82.4 mg, 638 μmol, 111 μL, 1.0 eq). The mixture was stirred at 25° C. for 2 h. LCMS indicated that the starting material was completely consumed, and the desired mass was detected. The mixture was poured into H₂O (10 mL) and extracted with EtOAc (10 mL×8). The combined organic layers were dried over Na₂SO₄, filtered, and concentrated under vacuum to give a residue which was purified by preparative HPLC (Phenomenex Luna C18 column (100×30 mm, 5 μm); flow rate: 25 mL/min; gradient: 1%-25% B over 8 min; mobile phase A: 0.1% aqueous TFA, mobile phase B: acetonitrile). (S)—N-(1-(7-Hydroxy-2-methylquinolin-5-yl)cyclopropyl)-2-methyl-5-((1-methylazetidin-2-yl)methoxy)benzamide (80.0 mg, 147 μmol, 23% yield, TFA salt) was obtained as a yellow solid. M+H⁺=432.2 (LCMS); ¹H NMR (400 MHZ, DMSO-d₆) δ 9.40 (br d, J=8.5 Hz, 1H), 9.23-9.17 (m, 1H), 7.72 (d, J=8.6 Hz, 1H), 7.62 (d, J=2.1 Hz, 1H), 7.31 (d, J=1.9 Hz, 1H), 7.11 (d, J=8.5 Hz, 1H), 6.99-6.90 (m, 1H), 6.80-6.73 (m, 1H), 4.68-4.56 (m, 1H), 4.23 (d, J=5.3 Hz, 2H), 4.08-3.99 (m, 1H), 3.92-3.81 (m, 1H), 2.84 (d, J=3.1 Hz, 6H), 2.44-2.28 (m, 2H), 2.00-1.91 (m, 3H), 1.38 (br s, 2H), 1.29-1.12 (m, 2H).

### Example 430: (S)-2-Methyl-5-((1-methylazetidin-2-yl)methoxy)-N-(1-(7-(2,2,2-trifluoroethoxy)quinolin-5-yl)cyclopropyl)benzamide (Compound 795)

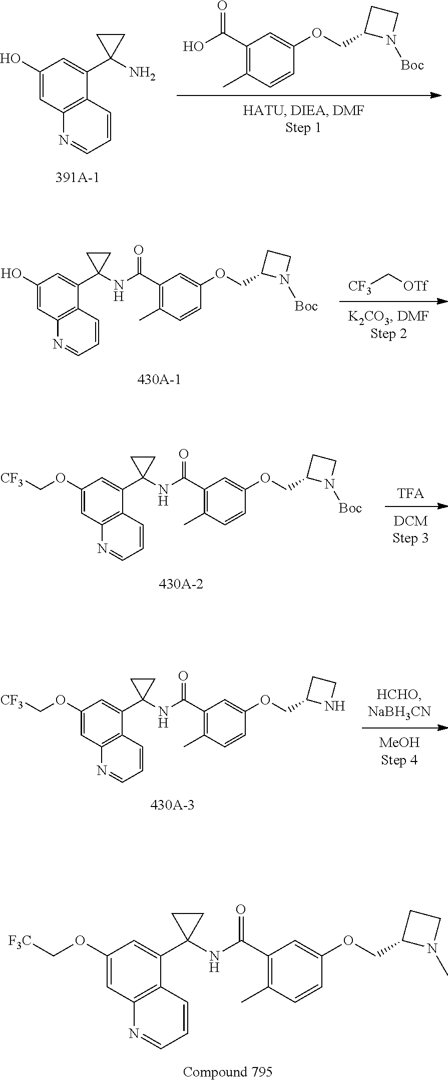

### Step 1: (S)-tert-Butyl 2-((3-((1-(7-hydroxyquinolin-5-yl)cyclopropyl)carbamoyl)-4-methylphenoxy)methyl)azetidine-1-carboxylate (430A-1)

To a mixture of 5-(1-aminocyclopropyl)quinolin-7-ol (300 mg, 1.50 mmol, 1.0 eq) and(S)-5-((1-(tert-butoxycarbonyl)azetidin-2-yl)methoxy)-2-methylbenzoic acid (337 mg, 1.05 mmol, 0.7 eq) in DMF (5.0 mL) were added DIEA (194 mg, 1.50 mmol, 261 μL, 1.0 eq) and HATU (399 mg, 1.05 mmol, 0.7 eq). The mixture was stirred at 20° C. for 16 h. LCMS indicated that the starting material was completely consumed, and the desired mass was detected. The mixture was poured into H₂O (10 mL) and extracted with EtOAc (10 mL×8). The combined organic layers were dried over Na₂SO₄, filtered, and concentrated under vacuum to give a residue which was purified by flash silica gel chromatography using a gradient of petroleum ether/EtOAc from 1/0 to 0/1. (S)-tert-Butyl 2-((3-((1-(7-hydroxyquinolin-5-yl)cyclopropyl)carbamoyl)-4-methylphenoxy)methyl)azetidine-1-carboxylate (450 mg, 894 μmol, 60% yield) was obtained as a yellow solid. M+H⁺=504.2 (LCMS).

### Step 2: tert-Butyl(S)-2-((4-Methyl-3-((1-(7-(2,2,2-trifluoroethoxy)quinolin-5-yl)cyclopropyl)carbamoyl)phenoxy)methyl)azetidine-1-carboxylate (430A-2)

To a mixture of tert-butyl(S)-2-((3-((1-(7-hydroxyquinolin-5-yl)cyclopropyl)carbamoyl)-4-methylphenoxy)methyl)azetidine-1-carboxylate (200 mg, 397 μmol, 1.0 eq) and 2,2,2-trifluoroethyl trifluoromethanesulfonate (92.2 mg, 397 μmol, 1.0 eq) in DMF (10 mL) was added K₂CO₃ (110 mg, 794 μmol, 2.0 eq). The mixture was degassed and purged with N₂ three times, and the resulting mixture was stirred at 20° C. for 16 h under a N₂ atmosphere. LCMS indicated that the starting material was completely consumed, and the desired mass was detected. The reaction mixture was allowed to cool to room temperature, poured into H₂O (10 mL) and extracted with DCM (10 mL×2). The combined organic layers were dried over Na₂SO₄, filtered, and concentrated under vacuum to give a residue which was purified by flash silica gel chromatography using a gradient of petroleum ether/EtOAc from 1/0 to 2/1. tert-Butyl(S)-2-((4-methyl-3-((1-(7-(2,2,2-trifluoroethoxy)quinolin-5yl)cyclopropyl)carbamoyl) phenoxy)methyl)azetidine-1-carboxylate (100 mg, 171 μmol, 43% yield) was obtained as a white solid. M+H⁺=586.4 (LCMS).

### Step 3: (S)-5-(Azetidin-2-ylmethoxy)-2-methyl-N-(1-(7-(2,2,2-trifluoroethoxy)quinolin-5-yl)cyclopropyl)benzamide (430A-3)

To a solution of tert-butyl(S)-2-((4-methyl-3-((1-(7-(2,2,2-trifluoroethoxy)quinolin-5yl)cyclopropyl)carbamoyl)phenoxy)methyl)azetidine-1-carboxylate (100 mg, 171 μmol, 43% yield) in DCM (5.0 mL) was added TFA (195 mg, 1.71 mmol, 126 μL, 10 eq). The mixture was stirred at 20° C. for 1 h. LCMS indicated that the starting material was completely consumed, and the desired mass was detected. The mixture was concentrated under vacuum at 20° C. to give(S)-5-(azetidin-2-ylmethoxy)-2-methyl-N-(1-(7-(2,2,2-trifluoroethoxy)quinolin-5-yl)cyclopropyl)benzamide (80 mg, crude, TFA salt) as a yellow oil, which was used in the next step without any further purification. M+H⁺=486.4 (LCMS)

### Step 4: (S)-5-(Azetidin-2-ylmethoxy)-2-methyl-N-(1-(7-(2,2,2-trifluoroethoxy)quinolin-5-yl)cyclopropyl)benzamide (Compound 795)

To a solution of(S)-5-(Azetidin-2-ylmethoxy)-2-methyl-N-(1-(7-(2,2,2-trifluoroethoxy) quinolin-5-yl)cyclopropyl)benzamide (80.0 mg, crude, TFA salt) in MeOH (5.0 mL) was added TEA (50.0 μL), followed by formaldehyde (27.0 mg, 330 μmol, 24.5 μL, 37% purity in water, 2.0 eq). The resulting mixture was adjusted to pH 6 with a small amount of AcOH. The mixture was stirred at 20° C. for 30 min, then NaBH₃CN (20.7 mg, 330 μmol, 2.0 eq) was added. The resulting reaction mixture was stirred at 20° C. for another 1 h. LCMS indicated that the starting material was completely consumed, and the desired mass was detected. The mixture was treated with H₂O (5.0 mL) and extracted with DCM (1.0 mL×5). The combined organic layers were dried over Na₂SO₄, filtered, and concentrated under vacuum to give a residue which was purified by preparative HPLC (Phenomenex Luna C18 column (100× 40 mm, 5 μm); flow rate: 25 mL/min; gradient: 15%-45% B over 8 min; mobile phase A: 0.1% aqueous TFA, mobile phase B: acetonitrile). (S)-5-(Azetidin-2-ylmethoxy)-2-methyl-N-(1-(7-(2,2,2-trifluoroethoxy)quinolin-5-yl)cyclopropyl)benzamide (23.7 mg, 23% yield, TFA salt) was obtained as a white solid. M+H⁺=500.4 (LCMS); ¹H NMR (400 MHZ, DMSO-d₆) δ 9.94-9.82 (m, 1H), 9.20-9.12 (m, 1H), 9.03 (d, J=8.6 Hz, 1H), 8.90 (dd, J=1.3, 4.2 Hz, 1H), 7.59-7.50 (m, 3H), 7.10 (d, J=8.4 Hz, 1H), 6.92 (dd, J=2.6, 8.4 Hz, 1H), 6.72 (d, J=2.6 Hz, 1H), 4.98 (q, J=8.8 Hz, 2H), 4.64-4.57 (m, 1H), 4.25-4.20 (m, 2H), 4.07-4.00 (m, 1H), 3.87 (br dd, J=6.3, 9.8 Hz, 1H), 2.84 (d, J=5.0 Hz, 3H), 2.41-2.30 (m, 2H), 1.95 (s, 3H), 1.35 (br s, 2H), 1.25 (br s, 2H).

### Example 431: (S)-2-Methyl-N-(1-(2-methyl-7-(3,3,3-trifluoropropyl)quinolin-5-yl)cyclopropyl)-5-((1-methylazetidin-2-yl)methoxy)benzamide (Compound 815)

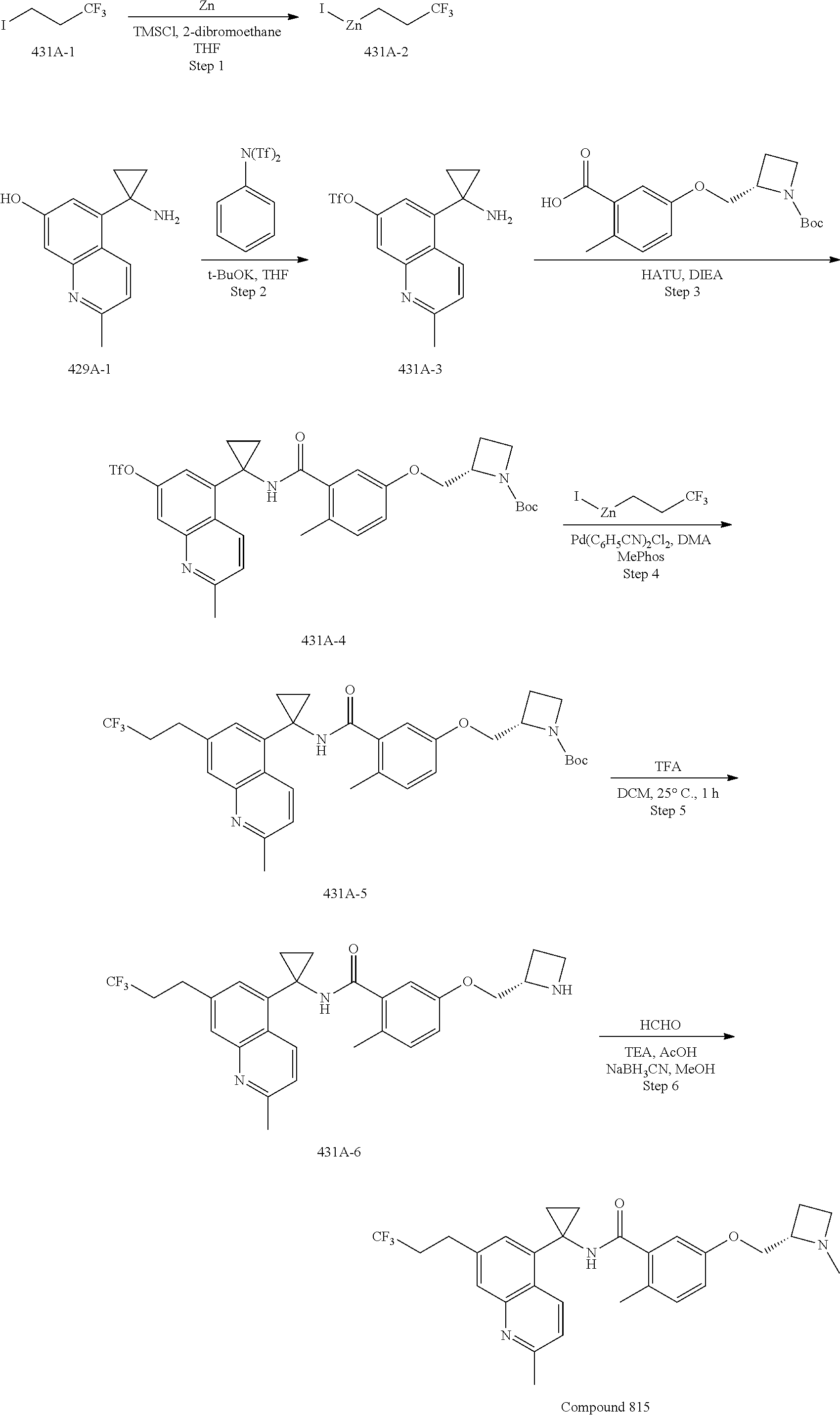

### Step 1: (3,3,3-Trifluoropropyl) zinc (II) iodide (431A-2)

To a solution of Zinc powder (876 mg, 13.4 mmol, 3.0 eq) in THF (3.0 mL) was added 1,2-dibromoethane (178 mg, 939 μmol, 71.7 μL, 0.21 eq). The mixture was stirred at 70° C. for 10 min under a N₂ atmosphere, then cooled to 25° C., and TMSCl (28.3 mg, 260 μmol, 33.0 μL, 0.06 eq) was added. The mixture was stirred at 25° C. for 10 min. A solution of 1,1,1-trifluoro-3-iodopropane (1.00 g, 4.47 mmol, 524 μL, 1.0 eq) in THF (2.0 mL) was added dropwise. The mixture was stirred at 25° C. until the reaction mixture turned gray. The mixture was used in the next step without any further purification.

### Step 2: 5-(1-Aminocyclopropyl)-2-methylquinolin-7-yl trifluoromethanesulfonate (431A-3)

To a solution of 5-(1-aminocyclopropyl)-2-methylquinolin-7-ol (1.40 g, 6.53 mmol, 1.0 eq) in THF (60 mL) was added t-BuOK (1.47 g, 13.1 mmol, 2.0 eq) at 0° C. under a N₂ atmosphere. The mixture was stirred at 0° C. for 15 min. 1,1,1-Trifluoro-N-phenyl-N-(trifluoromethylsulfonyl)methanesulfonamide (4.67 g, 13.1 mmol, 2.0 eq) was added in portions. The resulting reaction mixture was stirred at 20° C. for 15 h. LCMS indicated that the starting material was completely consumed, and the desired mass was detected. The reaction mixture was poured into H₂O (50 mL) and extracted with EtOAc (50 mL×3). The combined organic layers were dried over Na₂SO₄, filtered, and concentrated under vacuum to give a residue which was purified by flash silica gel chromatography using a gradient of EtOAc/petroleum ether from 0/1 to 1/1. 5-(1-Aminocyclopropyl)-2-methylquinolin-7-yl trifluoromethanesulfonate (850 mg, 2.45 mmol, 53% yield) was obtained as a yellow oil. M+H⁺=333.2 (LCMS); ¹H NMR (400 MHZ, CD₃OD) δ 8.88 (d, J=8.8 Hz, 1H), 7.83 (d, J=2.4 Hz, 1H), 7.63-7.57 (m, 2H), 2.76 (s, 3H), 1.25 (d, J=7.3 Hz, 2H), 1.07-1.03 (m, 2H).

### Step 3: tert-Butyl(S)-2-((4-methyl-3-((1-(2-methyl-7-(((trifluoromethyl)sulfonyl)oxy)quinolin-5-yl)cyclopropyl)carbamoyl)phenoxy)methyl)azetidine-1-carboxylate (431A-4)

To a solution of 5-(1-aminocyclopropyl)-2-methylquinolin-7-yl trifluoromethanesulfonate (850 mg, 2.45 mmol 1.0 eq) and(S)-5-((1-(tert-butoxycarbonyl)azetidin-2-yl)methoxy)-2-methylbenzoic acid (789 mg, 2.45 mmol, 1.0 eq) in DMF (20 mL) were added DIEA (952 mg, 7.36 mmol, 1.28 mL, 3.0 eq) and HATU (2.33 g, 6.14 mmol, 2.5 eq). The mixture was stirred at 20° C. for 12 h. LCMS indicated that the starting material was completely consumed, and the desired mass was detected. The reaction mixture was poured into H₂O (50 mL) and extracted with EtOAc (30 mL×5). The combined organic layers were dried over Na₂SO₄, filtered, and concentrated under vacuum to give a residue which was purified by flash silica gel chromatography using a gradient of EtOAc/petroleum ether from 1/100 to 1/0. tert-Butyl(S)-2-((4-methyl-3-((1-(2-methyl-7-(((trifluoromethyl)sulfonyl)oxy)quinolin-5-yl)cyclopropyl) carbamoyl)phenoxy)methyl)azetidine-1-carboxylate (1.30 g, 2.00 mmol, 82% yield) was obtained as a yellow gum. M+H⁺=650.3 (LCMS); ¹H NMR (400 MHZ, DMSO-d₆) δ 9.20 (s, 1H), 9.01 (d, J=8.7 Hz, 1H), 7.95 (d, J=2.4 Hz, 1H), 7.80 (d, J=2.6 Hz, 1H), 7.62 (d, J=8.8 Hz, 1H), 7.05 (d, J=8.4 Hz, 1H), 6.89 (dd, J=2.7, 8.3 Hz, 1H), 6.68 (d, J=2.6 Hz, 1H), 4.44-4.32 (m, 1H), 4.15 (dd, J=4.8, 10.3 Hz, 1H), 4.01-3.96 (m, 1H), 3.74 (br d, J=5.7 Hz, 2H), 2.70 (s, 3H), 2.30-2.24 (m, 1H), 2.10-2.03 (m, 1H), 1.94 (s, 3H), 1.42-1.26 (m, 13H).

### Step 4: tert-Butyl(S)-2-((4-methyl-3-((1-(2-methyl-7-(3,3,3-trifluoropropyl)quinolin-5-yl)cyclopropyl)carbamoyl)phenoxy)methyl)azetidine-1-carboxylate (431A-5)

To a solution of tert-butyl(S)-2-((4-methyl-3-((1-(2-methyl-7-(((trifluoromethyl) sulfonyl)oxy)quinolin-5-yl)cyclopropyl)carbamoyl)phenoxy)methyl)azetidine-1-carboxylate (150 mg, 231 μmol, 1.0 eq) and (3,3,3-trifluoropropyl) zinc (II) iodide (1 M, 693 μL, 3.0 eq) in DMA (8.0 mL) were added Pd(C₆H₅CN)₂Cl₂ (4.43 mg, 11.5 μmol, 0.05 eq) and MePhos (8.42 mg, 23.1 μmol, 0.1 eq). The mixture was stirred at 60° C. for 16 h under a N₂ atmosphere. LCMS indicated that the starting material was completely consumed, and the desired mass was detected. The reaction mixture was allowed to cool to room temperature, poured into H₂O (20 mL), and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 0/1 to 1/0. tert-Butyl(S)-2-((4-methyl-3-((1-(2-methyl-7-(3,3,3-trifluoropropyl)quinolin-5-yl)cyclopropyl) carbamoyl)phenoxy)methyl)azetidine-1-carboxylate (120 mg, 200 μmol) was obtained as a brown oil. M+H⁺=598.3 (LCMS); ¹H NMR (400 MHZ, DMSO-d₆) δ 9.06 (s, 1H), 8.92 (d, J=8.8 Hz, 1H), 7.72 (d, J=4.0 Hz, 2H), 7.42 (d, J=8.6 Hz, 1H), 7.04 (d, J=8.5 Hz, 1H), 6.87 (dd, J=2.5, 8.4 Hz, 1H), 6.64 (d, J=2.5 Hz, 1H), 4.43-4.33 (m, 1H), 4.14 (dd, J=4.7, 9.9 Hz, 1H), 4.02-3.95 (m, 1H), 3.78-3.67 (m, 2H), 3.06-2.99 (m, 2H), 2.73-2.68 (m, 2H), 2.30-2.22 (m, 1H), 2.12-2.04 (m, 1H), 1.36-1.27 (m, 11H), 1.20 (br d, J=4.1 Hz, 2H).

### Step 5: (S)-5-(Azetidin-2-ylmethoxy)-2-methyl-N-(1-(2-methyl-7-(3,3,3-trifluoropropyl)quinolin-5-yl)cyclopropyl)benzamide (431A-6)

To a solution of tert-butyl(S)-2-((4-methyl-3-((1-(2-methyl-7-(3,3,3-trifluoropropyl)quinolin-5-yl)cyclopropyl)carbamoyl)phenoxy)methyl)azetidine-1-carboxylate (110 mg, 184 μmol, 1.0 eq) in DCM (5.0 mL) was added TFA (1.54 g, 13.5 mmol, 1.0 mL, 73.4 eq). The mixture was stirred at 20° C. for 1 h. LCMS indicated that the starting material was completely consumed, and the desired mass was detected. The reaction mixture was concentrated under vacuum at 20° C. to give the crude(S)-5-(azetidin-2-ylmethoxy)-2-methyl-N-(1-(2-methyl-7-(3,3,3-trifluoro propyl)quinolin-5-yl)cyclopropyl)benzamide (110 mg, 180 μmol, TFA salt) as a brown oil. M+H⁺=498.2 (LCMS).

### Step 6: (S)-2-Methyl-N-(1-(2-methyl-7-(3,3,3-trifluoropropyl)quinolin-5-yl)cyclopropyl)-5-((1-methylazetidin-2-yl)methoxy)benzamide (Compound 815)

To a solution of(S)-5-(azetidin-2-ylmethoxy)-2-methyl-N-(1-(2-methyl-7-(3,3,3-trifluoropropyl)quinolin-5-yl)cyclopropyl)benzamide (110 mg, 180 μmol, 1.0 eq, TFA salt) in MeOH (5.0 mL) was added TEA (0.1 mL), followed by formaldehyde (110 mg, 1.36 mmol, 101 μL, 37% purity in water, 7.5 eq). The resulting mixture was adjusted to pH 6 with a small amount of AcOH. The mixture was stirred at 20° C. for 30 min, then NaBH₃CN (33.9 mg, 540 μmol, 3.0 eq) was added. The reaction mixture was stirred at 20° C. for another 1 h. LCMS indicated that the starting material was completely consumed, and the desired mass was detected. The reaction mixture was diluted with saturated aqueous NaHCO₃ (100 μL) and concentrated under vacuum to give a residue which was purified by preparative HPLC (Phenomenex Luna column (80×30 mm, 3 μm); flow rate: 25 mL/min; gradient: 10%-40% B over 8 min; mobile phase A: 0.04% aqueous HCl, mobile phase B: acetonitrile). (S)-2-Methyl-N-(1-(2-methyl-7-(3,3,3-trifluoropropyl)quinolin-5-yl)cyclopropyl)-5-((1-methylazetidin-2-yl)methoxy)benzamide (50.1 mg, 97.2 μmol, 54% yield, HCl salt) was obtained as a yellow solid. M+H⁺=512.2 (LCMS); ¹H NMR (400 MHZ, DMSO-d₆) δ 11.50-11.21 (m, 1H), 9.74-9.61 (m, 1H), 9.45-9.33 (m, 1H), 8.22 (s, 1H), 8.08 (s, 1H), 8.02 (br d, J=8.8 Hz, 1H), 7.12-7.06 (m, 1H), 6.98-6.89 (m, 1H), 6.84-6.73 (m, 1H), 4.70-4.55 (m, 1H), 4.47 (dd, J=8.5, 11.0 Hz, 1H), 4.34-4.19 (m, 1H), 4.02-3.93 (m, 1H), 3.90-3.81 (m, 1H), 3.19-3.12 (m, 2H), 2.99 (s, 3H), 2.83-2.67 (m, 5H), 2.41-2.24 (m, 2H), 1.97 (s, 3H), 1.43 (br s, 2H), 1.32 (br s, 2H).

### Example 432: (S)—N-(1-(7-Cyclopentylquinolin-5-yl)cyclopropyl)-2-methyl-5-((1-methyl azetidin-2-yl)methoxy)benzamide (Compound 648)

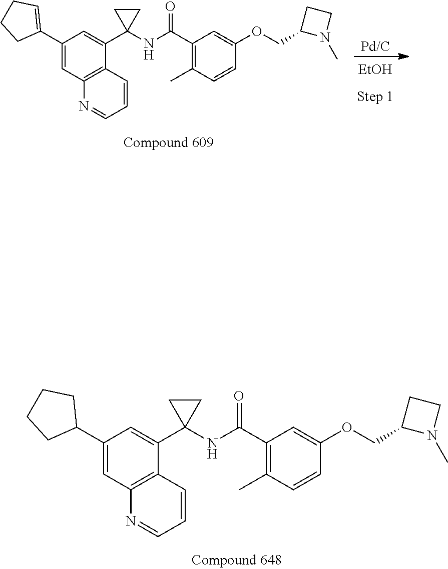 [SMILES: "EtOH" ; Cc1ccc(OC[C@@H]2CCN2C)cc1C(=O)NC1(c2cc(C3=CCCC3)cc3ncccc23)CC1 ; "Pd/C" ; Cc1ccc(OC[C@@H]2CCN2C)cc1C(=O)NC1(c2cc(C3CCCC3)cc3ncccc23)CC1]

### Step 1: (S)—N-(1-(7-Cyclopentylquinolin-5-yl)cyclopropyl)-2-methyl-5-((1-methylazetidin-2-yl)methoxy)benzamide (Compound 648)

To a solution of(S)—N-(1-(7-(cyclopent-1-en-1-yl)quinolin-5-yl)cyclopropyl)-2-methyl-5-((1-methylazetidin-2-yl)methoxy)benzamide (90.0 mg, 192 μmol, 1.0 eq) in EtOH (10 mL) was added 10% palladium on carbon (90.0 mg) under a N₂ atmosphere. The suspension was degassed and purged with H₂ three times. The resulting mixture was stirred at 20° C. for 16 h under a H₂ (15 psi) atmosphere. LCMS indicated that the starting material was completely consumed, and the desired mass was detected. The suspension was filtered through a pad of Celite and the pad was washed with EtOH (5.0 mL×3). The combined filtrates were concentrated under vacuum to give a residue which was purified by preparative HPLC (Phenomenex Luna C18 (80×30 mm, 3 μm); flow rate: 25 mL/min; gradient: 15%-45% B over 8 min; mobile phase A: 0.04% aqueous HCl, mobile phase B: acetonitrile). (S)—N-(1-(7-Cyclopentylquinolin-5-yl)cyclopropyl)-2-methyl-5-((1-methylazetidin-2-yl)methoxy)benzamide (3.00 mg, 5.99 μmol, 3% yield, HCl salt) was obtained as a white solid. M+H⁺=570.2 (LCMS); ¹H NMR (400 MHZ, DMSO-d₆) δ 10.12-9.95 (m, 1H), 9.16-9.12 (m, 1H), 9.05-8.88 (m, 1H), 7.89 (s, 1H), 7.85-7.80 (m, 1H), 7.73-7.60 (m, 1H), 7.13-7.05 (m, 1H), 6.98-6.86 (m, 1H), 6.68 (s, 1H), 4.78-4.44 (m, 1H), 4.24 (br d, J=7.7 Hz, 2H), 4.07-3.92 (m, 1H), 3.91-3.70 (m, 1H), 2.84-2.82 (m, 2H), 2.64-2.59 (m, 2H), 2.16-2.11 (m, 2H), 2.09-2.07 (m, 2H), 2.00-1.96 (m, 3H), 1.88-1.81 (m, 2H), 1.75-1.64 (m, 4H), 1.43-1.33 (m, 2H), 1.24 (br s, 2H).

### Example 433: (S)-2-Methyl-5-((1-methylazetidin-2-yl)methoxy)-N-(1-(7-(pyrrolidin-1-yl)quinolin-5-yl)cyclopropyl)benzamide (Compound 683)

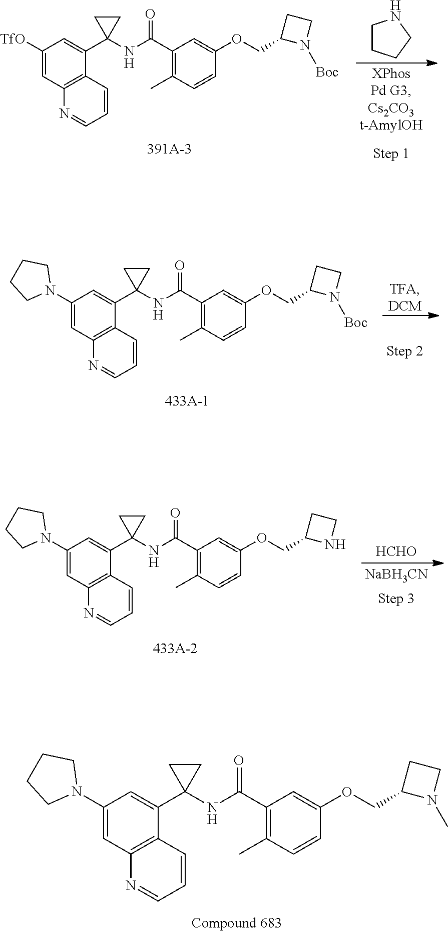

### Step 1: tert-Butyl(S)-2-((4-methyl-3-((1-(7-(pyrrolidin-1-yl)quinolin-5-yl)cyclopropyl)carbamoyl)phenoxy)methyl)azetidine-1-carboxylate (433A-1)

To a solution of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (100 mg, 157 μmol, 1.0 eq) and pyrrolidine (13.4 mg, 189 μmol, 15.8 μL, 1.2 eq) in 2-methylbutan-2-ol (2.0 mL) were added Cs₂CO₃ (103 mg, 315 μmol, 2.0 eq) and XPhos Pd G3 (66.6 mg, 78.7 μmol, 0.5 eq) at 20° C. The mixture was stirred at 80° C. for 12 h under a N₂ atmosphere. LCMS indicated that the starting material was completely consumed. The mixture was allowed to cool to room temperature, treated with water (10 mL) and extracted with DCM (10 mL×2). The combined organic layers were dried over Na₂SO₄, filtered, and concentrated under vacuum to give a residue which was purified by flash silica gel chromatography using a gradient of EtOAc/petroleum ether from 0/1 to 1/0. tert-Butyl(S)-2-((4-methyl-3-((1-(7-(pyrrolidin-1-yl)quinolin-5-yl)cyclopropyl)carbamoyl)phenoxy)methyl)azetidine-1-carboxylate (150 mg, 75.5 μmol, 48% yield) was obtained as a yellow oil. M+H⁺=557.4 (LCMS).

### Step 2: (S)-5-(Azetidin-2-ylmethoxy)-2-methyl-N-(1-(7-(pyrrolidin-1-yl)quinolin-5-yl)cyclopropyl)benzamide (433A-2)

To a solution of tert-butyl(S)-2-((4-methyl-3-((1-(7-(pyrrolidin-1-yl)quinolin-5-yl)cyclopropyl)carbamoyl)phenoxy)methyl)azetidine-1-carboxylate (150 mg, 269 μmol, 1.0 eq) in DCM (2.0 mL) was added TFA (1.54 g, 13.5 mmol, 1.0 mL, 50 eq). The mixture was stirred at 20° C. for 1 h. LCMS indicated that the starting material was completely consumed, and the desired mass was detected. The reaction mixture was concentrated under vacuum at 30° C. to give(S)-5-(azetidin-2-ylmethoxy)-2-methyl-N-(1-(7-(5-(pyrrolidin-1-ylmethyl)thiophen-2-yl)quinolin-5-yl)cyclopropyl)benzamide (170 mg, TFA salt) as a yellow oil, which was used in the next step without any further purification. M+H⁺=457.3 (LCMS).

### Step 3: (S)-2-Methyl-5-((1-methylazetidin-2-yl)methoxy)-N-(1-(7-(pyrrolidin-1-yl)quinolin-5-yl)cyclopropyl)benzamide (Compound 683)

To solution of(S)-5-(azetidin-2-ylmethoxy)-2-methyl-N-(1-(7-(5-(pyrrolidin-1-a ylmethyl)thiophen-2-yl)quinolin-5-yl)cyclopropyl)benzamide (150 mg, 328 μmol, 1.0 eq) in MeOH (2.0 mL) were added HOAc (1.85 mg, 30.0 μmol, 1.76 μL, 0.1 eq) and formaldehyde (37.4 mg, 461 μmol, 34.4 μL, 37% purity in H₂O, 1.5 eq) at 20° C. for 1 h. Then NaBH₃CN (48.3 mg, 768 μmol, 2.5 eq) was added to the mixture. The mixture was stirred at 20° C. for 1 h. LCMS indicated that the starting material was completely consumed, and the desired mass was detected. The mixture was concentrated under vacuum at 30° C. to give a residue which was purified by preparative HPLC ((Phenomenex Gemini C18 column (150×40 mm, 10 μm); flow rate: 25 mL/min; gradient: 30%-60% B over 8 min; mobile phase A: 10 mM aqueous NH₄HCO₃, mobile phase B: acetonitrile)). (S)-2-Methyl-5-((1-methylazetidin-2-yl)methoxy)-N-(1-(7-(pyrrolidin-1-yl)quinolin-5-yl)cyclopropyl)benzamide (3.00 mg, 6.37 μmol, 2% yield) was obtained as a white solid. M+H⁺=471.3 (LCMS); ¹H NMR (400 MHZ, DMSO-d₆) δ 9.07-9.00 (m, 1H), 8.75-8.83 (m, 1H), 8.64 (m, 1H), 7.38 (m, 1H), 7.15 (m, 1H), 6.98-7.06 (m, 1H), 6.83 (m, 1H), 6.75 (m, 1H), 6.59 (m, 1H), 3.82-3.90 (m, 2H), 3.40 (m, 4H), 3.17-3.25 (m, 3H), 2.69-2.76 (m, 1H), 2.17-2.24 (m, 3H), 1.91-2.05 (m, 8H), 1.80-1.89 (m, 1H), 1.29-1.38 (m, 2H), 1.12-1.21 (m, 2H).

### Example 434: 2-Methyl-5-(2-(methylamino)ethoxy)-N-(1-(7-(thiophen-2-yl)quinolin-5-yl)cyclopropyl)benzamide (Compound 630)

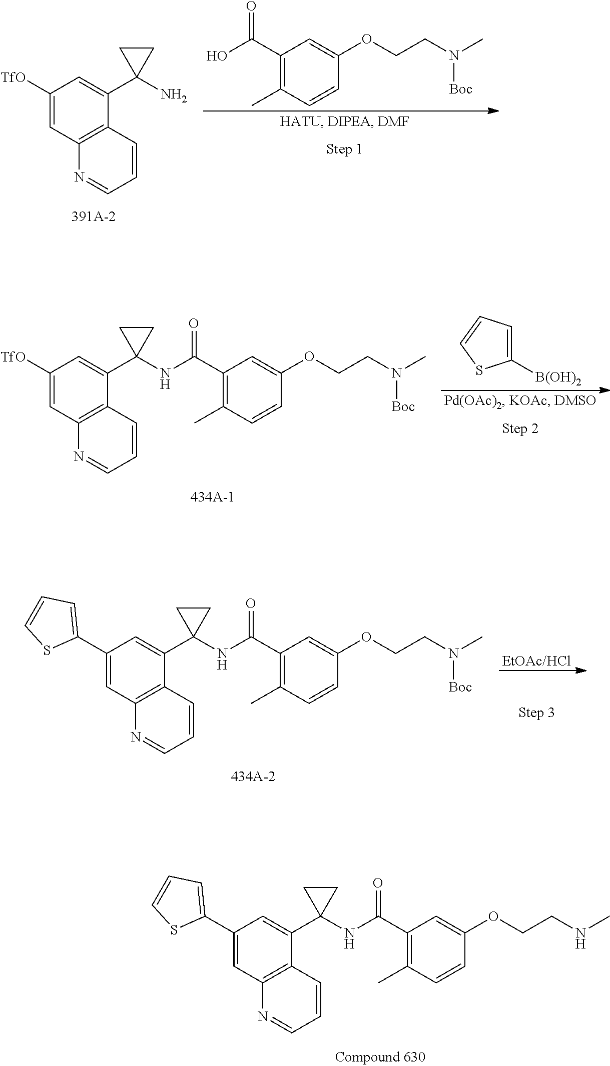

### Step 1: 5-(1-(5-(2-((tert-Butoxycarbonyl)(methyl)amino)ethoxy)-2-methylbenzamido)cyclopropyl)quinolin-7-yl trifluoromethanesulfonate (434A-1)

To a solution of 5-(1-aminocyclopropyl)quinolin-7-yl trifluoromethanesulfonate (200 mg, 602 μmol, 1.0 eq) and 5-(2-((tert-butoxycarbonyl)(methyl)amino)ethoxy)-2-methylbenzoic acid (186 mg, 602 μmol, 1.0 eq) in DMF (5.0 mL) were added DIEA (233 mg, 1.81 mmol, 315 μL, 3.0 eq) and HATU (572 mg, 1.50 mmol, 2.5 eq). The mixture was stirred at 20° C. for 14 h. LCMS indicated that the starting material was completely consumed, and the desired mass was detected. The reaction mixture was poured into H₂O (10 mL) and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 0/1 to 7/3. 5-(1-(5-(2-((tert-Butoxycarbonyl)(methyl)amino)ethoxy)-2-methylbenzamido)cyclopropyl)quinolin-7-yl trifluoromethane sulfonate (260 mg, 417 μmol, 69% yield) was obtained as a brown oil. M+H⁺=624.2 (LCMS); ¹H NMR (400 MHZ, DMSO-d₆) δ 9.22 (s, 1H), 9.14 (br d, J=8.6 Hz, 1H), 9.08-9.01 (m, 1H), 8.08 (d, J=2.5 Hz, 1H), 7.88 (d, J=2.5 Hz, 1H), 7.73 (dd, J=4.1, 8.6 Hz, 1H), 7.04 (br d, J=8.4 Hz, 1H), 6.85 (dd, J=2.5, 8.4 Hz, 1H), 6.63 (br s, 1H), 3.98 (br t, J=5.8 Hz, 2H), 3.46 (br t, J=5.6 Hz, 2H), 2.86-2.74 (m, 3H), 1.93 (s, 3H), 1.44-1.23 (m, 13H).

### Step 2: tert-Butyl methyl(2-(4-methyl-3-((1-(7-(thiophen-2-yl)quinolin-5-yl)cyclopropyl)carbamoyl)phenoxy)ethyl)carbamate (434A-2)

To a solution of 5-(1-(5-(2-((tert-butoxycarbonyl)(methyl)amino)ethoxy)-2-methylbenzamido)cyclopropyl)quinolin-7-yl trifluoromethanesulfonate (200 mg, 320.70 μmol, 1.0 eq) and thiophen-2-ylboronic acid (82.1 mg, 641 μmol, 2.0 eq) in DMSO (5.0 mL) were added KOAc (94.4 mg, 962 μmol, 3.0 eq), Pd(OAc)₂ (14.4 mg, 64.1 μmol, 0.2 eq) and cataCxium A (46.0 mg, 128 μmol, 0.4 eq). The mixture was degassed and purged with N₂ three times, and the mixture was stirred at 80° C. for 15 h under a N₂ atmosphere. LCMS indicated that the starting material was completely consumed, and the desired mass was detected. The reaction mixture was allowed to cool to room temperature, poured into H₂O (20 mL) and extracted with EtOAc (20 mL×3). The combined organic layers were dried over Na₂SO₄, filtered, and concentrated under vacuum to give a residue which was purified by flash silica gel chromatography using a gradient of EtOAc/petroleum ether from 0/1 to 7/3. tert-Butyl methyl(2-(4-methyl-3-((1-(7-(thiophen-2-yl)quinolin-5-yl)cyclopropyl)carbamoyl) phenoxy)ethyl)carbamate (180 mg, 323 μmol, crude) was obtained as a brown oil. M+H⁺=558.3 (LCMS); ¹H NMR (400 MHZ, DMSO-d₆) δ 9.15 (s, 1H), 9.04 (br d, J=8.5 Hz, 1H), 8.92 (br d, J=3.4 Hz, 1H), 8.15 (s, 2H), 7.78 (d, J=3.3 Hz, 1H), 7.68 (d, J=5.0 Hz, 1H), 7.56 (dd, J=4.1, 8.5 Hz, 1H), 7.31-7.17 (m, 1H), 7.04 (br d, J=8.3 Hz, 1H), 6.84 (dd, J=2.3, 8.3 Hz, 1H), 6.61 (br s, 1H), 4.00-3.91 (m, 2H), 3.46 (br t, J=5.5 Hz, 2H), 2.81 (br d, J=8.0 Hz, 3H), 1.96 (s, 3H), 1.49-1.25 (m, 13H).

### Step 3: 2-Methyl-5-(2-(methylamino)ethoxy)-N-(1-(7-(thiophen-2-yl)quinolin-5-yl)cyclopropyl)benzamide (Compound 630)

To a solution tert-butyl methyl(2-(4-methyl-3-((1-(7-(thiophen-2-yl)quinolin-5-yl)cyclopropyl)carbamoyl)phenoxy)ethyl)carbamate (180 mg, 323 μmol, 1.0 eq) in EtOH (2.0 mL) was added HCl/EtOAc (4 M, 5.0 mL). The mixture was stirred at 20° C. for 1 h. LCMS indicated that the starting material was completely consumed, and the desired mass was detected. The reaction mixture was concentrated under vacuum to give a residue which was purified by preparative HPLC (Phenomenex Luna column (80×30 mm, 3 μm); flow rate: 25 mL/min; gradient: 1%-40% B over 8 min; mobile phase A: 0.04% aqueous HCl, mobile phase B: acetonitrile). 2-Methyl-5-(2-(methylamino)ethoxy)-N-(1-(7-(thiophen-2-yl)quinolin-5-yl)cyclopropyl)benzamide (70.1 mg, 141 μmol, 44% yield, HCl salt) was obtained as a yellow solid. M+H⁺=458.1 (LCMS); ¹H NMR (400 MHZ, DMSO-d₆) δ 9.46 (br d, J=8.4 Hz, 1H), 9.33 (s, 1H), 9.15 (d, J=4.0 Hz, 1H), 8.97 (br d, J=3.5 Hz, 2H), 8.33 (d, J=5.8 Hz, 2H), 7.94-7.84 (m, 2H), 7.79 (d, J=4.5 Hz, 1H), 7.29 (dd, J=3.8, 5.0 Hz, 1H), 7.09 (d, J=8.5 Hz, 1H), 6.91 (dd, J=2.7, 8.4 Hz, 1H), 6.72 (d, J=2.6 Hz, 1H), 4.17 (t, J=5.0 Hz, 2H), 3.26-3.21 (m, 2H), 2.59-2.55 (m, 3H), 1.98 (s, 3H), 1.44 (br s, 2H), 1.38 (br s, 2H).

### Example 435: (S)-5-(Azetidin-2-ylmethoxy)-2-methyl-N-(1-(7-(thiophen-2-yl)quinolin-5-yl)cyclopropyl)benzamide (Compound 608)

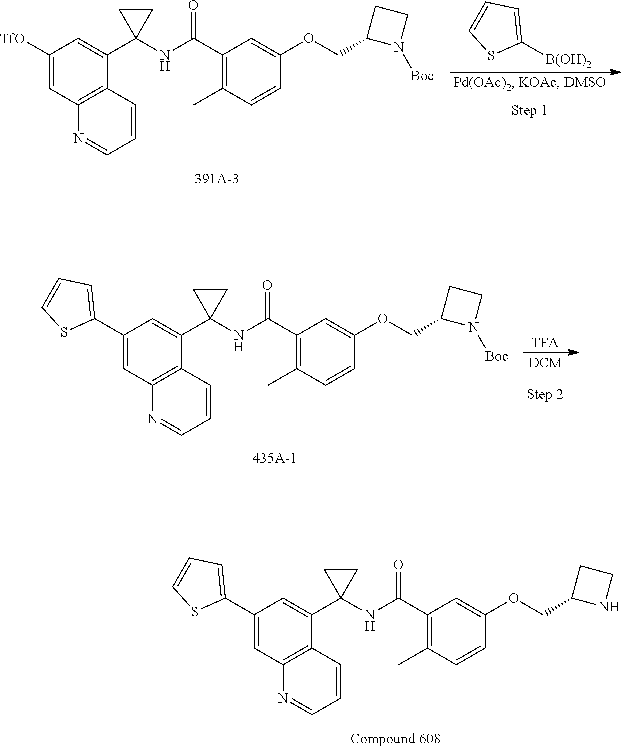

### Step 1: tert-Butyl(S)-2-((4-methyl-3-((1-(7-(thiophen-2-yl)quinolin-5-yl)cyclopropyl)carbamoyl)phenoxy)methyl)azetidine-1-carboxylate (435A-1)

To a solution of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (200 mg, 315 μmol, 1.0 eq) and thiophen-2-ylboronic acid (80.5 mg, 629 μmol, 2.0 eq) in DMSO (5.0 mL) were added KOAc (92.6 mg, 944 μmol, 3.0 eq), Pd(OAc)₂ (14.1 mg, 62.9 μmol, 0.2 eq) and cataCxium A (45.1 mg, 126 μmol, 0.4 eq). The resulting mixture was stirred at 80° C. for 13 h under a N₂ atmosphere. LCMS indicated that the starting material was completely consumed, and the desired mass was detected. The reaction mixture was allowed to cool to room temperature, poured into H₂O (30 mL) and extracted with EtOAc (10 mL×3). The combined organic layers were dried over Na₂SO₄, filtered, and concentrated under vacuum to give tert-butyl(S)-2-((4-methyl-3-((1-(7-(thiophen-2-yl)quinolin-5-yl)cyclopropyl)carbamoyl)phenoxy)methyl)azetidine-1-carboxylate (400 mg) as a brown oil, which was used in the next step without any further purification. M+H⁺=570.3 (LCMS).

### Step 2: (S)-5-(Azetidin-2-ylmethoxy)-2-methyl-N-(1-(7-(thiophen-2-yl)quinolin-5-yl)cyclopropyl)benzamide (Compound 608)

To a solution of tert-butyl(S)-2-((4-methyl-3-((1-(7-(thiophen-2-yl)quinolin-5-yl)cyclopropyl)carbamoyl)phenoxy)methyl)azetidine-1-carboxylate (150 mg, 263 μmol, 1.0 eq) in DCM (1.5 mL) was added TFA (540 mg, 4.74 mmol, 351 μL). The mixture was stirred at 20° C. for 1 h. LCMS indicated that the starting material was completely consumed, and the desired mass was detected. The mixture was concentrated under vacuum at 20° C. to give a residue which was purified by preparative HPLC (Phenomenex Luna C18 column (75× 30 mm, 3 μm); flow rate: 25 mL/min; gradient: 5%-40% B over 8 min; mobile phase A: 0.1% aqueous TFA, mobile phase B: acetonitrile). (S)-5-(Azetidin-2-ylmethoxy)-2-methyl-N-(1-(7-(thiophen-2-yl)quinolin-5-yl)cyclopropyl)benzamide (74.3 mg, 127 μmol, 48% yield, TFA salt) was obtained as a yellow solid. M+H⁺=470.1 (LCMS); ¹H NMR (400 MHZ, DMSO-d₆) δ 9.18 (s, 1H), 9.09 (d, J=8.8 Hz, 1H), 8.96 (dd, J=1.4, 4.1 Hz, 1H), 8.89-8.66 (m, 2H), 8.18 (d, J=2.0 Hz, 2H), 7.86-7.77 (m, 1H), 7.71 (d, J=5.1 Hz, 1H), 7.62 (dd, J=4.3, 8.5 Hz, 1H), 7.25 (dd, J=3.8, 4.9 Hz, 1H), 7.10 (d, J=8.5 Hz, 1H), 6.91 (dd, J=2.8, 8.3 Hz, 1H), 6.70 (d, J=2.6 Hz, 1H), 4.70-4.68 (m, 1H), 4.68-4.62 (m, 1H), 4.23 (dd, J=7.1, 11.1 Hz, 1H), 4.16-4.10 (m, 1H), 3.95-3.80 (m, 2H), 2.45-2.27 (m, 2H), 1.98 (s, 3H), 1.40 (br s, 2H), 1.33 (br s, 2H).

### Example 436: (S)—N-(1-(7-(5-(Hydroxymethyl)thiophen-2-yl)quinolin-5-yl)cyclopropyl)-2-methyl-5-((1-methylazetidin-2-yl)methoxy)benzamide (Compound 753)

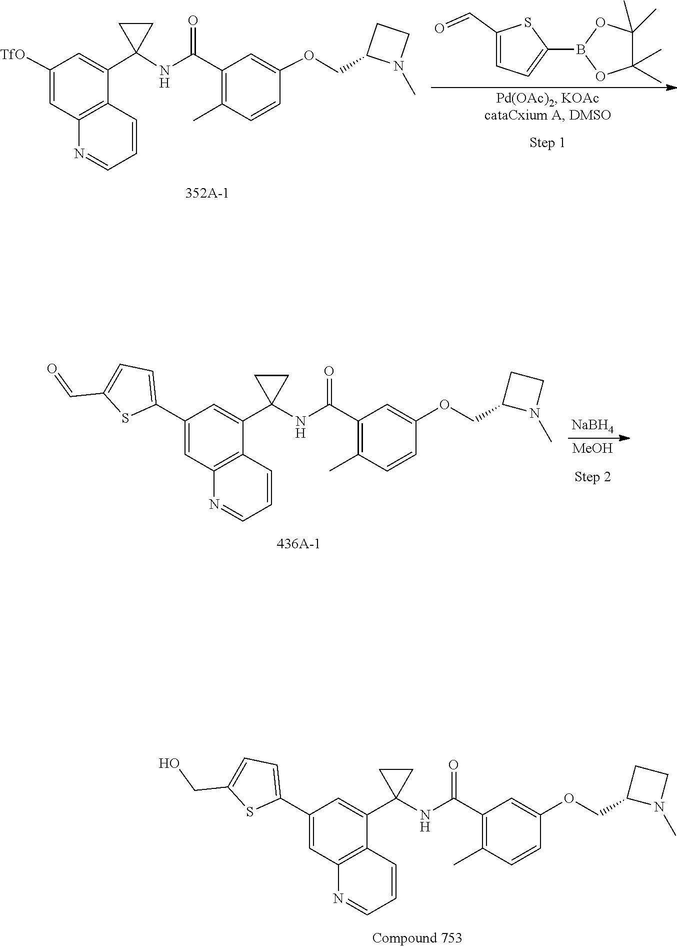

### Step 1: (S)—N-(1-(7-(5-Formylthiophen-2-yl)quinolin-5-yl)cyclopropyl)-2-methyl-5-((1-methylazetidin-2-yl)methoxy)benzamide (436A-1)

A mixture of(S)-5-(1-(2-methyl-5-((1-methylazetidin-2-yl)methoxy)benzamido)cyclopropyl) quinolin-7-yltrifluoromethanesulfonate (250 mg, 455 μmol, 1.0 eq), 5-(4,4,5,5-tetramethyl-1,3,2-dioxaborolan-2-yl)thiophene-2-carbaldehyde (142 mg, 910 μmol, 2.0 eq), KOAc (134 mg, 1.36 mmol, 3.0 eq), cataCxium A (32.6 mg, 91.0 μmol, 0.2 eq) and Pd(OAc)₂ (10.2 mg, 45.5 μmol, 0.1 eq) in DMSO (5.0 mL) was degassed and purged with N₂ three times. The mixture was stirred at 80° C. for 16 h under a N₂ atmosphere. LCMS indicated that the starting material was completely consumed, and the desired mass was detected. The reaction mixture was allowed to cool to room temperature, poured into H₂O (10 mL), and extracted with EtOAc (5.0 mL×3). The combined organic layers were dried over Na₂SO₄ filtered, and concentrated under vacuum to give a residue which was purified by flash silica gel chromatography using a gradient of EtOAc/petroleum ether from 0/1 to 1/5. (S)—N-(1-(7-(5-Formylthiophen-2-yl)quinolin-5-yl)cyclopropyl)-2-methyl-5-((1-methylazetidin-2-yl)methoxy)benzamide (230 mg, 450 μmol, 99% yield) was obtained as a yellow solid. M+H⁺=512.3 (LCMS).

### Step 2: (S)—N-(1-(7-(5-(Hydroxymethyl)thiophen-2-yl)quinolin-5-yl)cyclopropyl)-2-methyl-5-((1-methylazetidin-2-yl)methoxy)benzamide (Compound 753)

To a stirred solution of(S)—N-(1-(7-(5-formylthiophen-2-yl)quinolin-5-yl)cyclopropyl)-2-methyl-5-((1-methylazetidin-2-yl)methoxy)benzamide (40.0 mg, 78.2 μmol, 1.0 eq) in MeOH (2.0 mL) was added NaBH₄ (5.92 mg, 156 μmol, 2.0 eq). The reaction mixture was stirred at 25° C. for 1 h. LCMS indicated that the starting material was completely consumed, and the desired mass was detected. The reaction mixture was poured into H₂O (2.0 mL) and extracted with EtOAc (2.0 mL×3). The combined organic layers were dried over Na₂SO₄ filtered, and concentrated under vacuum to give a residue which was purified by preparative HPLC (Phenomenex Luna column (80×30 mm, 3 μm); flow rate: 25 mL/min; gradient: 5%-35% B over 8 min; mobile phase A: 0.04% aqueous HCl, mobile phase B: acetonitrile). (S)—N-(1-(7-(5-(Hydroxymethyl)thiophen-2-yl)quinolin-5-yl)cyclopropyl)-2-methyl-5-((1-methylazetidin-2-yl)methoxy)benzamide (19.0 mg, 33.1 μmol, 42% yield, HCl salt) was obtained as a yellow solid. M+H⁺=514.3 (LCMS); ¹H NMR (400 MHZ, DMSO-d₆) δ 10.50-10.21 (m, 1H), 9.45-9.20 (m, 2H), 9.13-8.96 (m, 1H), 8.32-8.17 (m, 2H), 7.85-7.63 (m, 2H), 7.15-7.03 (m, 2H), 7.00-6.91 (m, 1H), 6.92-6.89 (m, 1H), 6.75-6.71 (m, 1H), 4.74-4.68 (m, 2H), 4.64-4.61 (m, 1H), 4.65-4.58 (m, 1H), 4.32-4.17 (m, 2H), 4.06-3.98 (m, 1H), 3.88-3.82 (m, 1H), 2.83-2.75 (m, 3H), 2.36-2.28 (m, 2H), 2.01-1.94 (m, 4H), 1.47-1.34 (m, 4H), 1.29-1.21 (m, 2H).

### Example 437: 2-Methyl-5-(((S)-1-methylazetidin-2-yl)methoxy)-N-(1-(7-(5-(1-(pyrrolidin-1-yl)ethyl)thiophen-2-yl)quinolin-5-yl)cyclopropyl)benzamide (Compound 780)

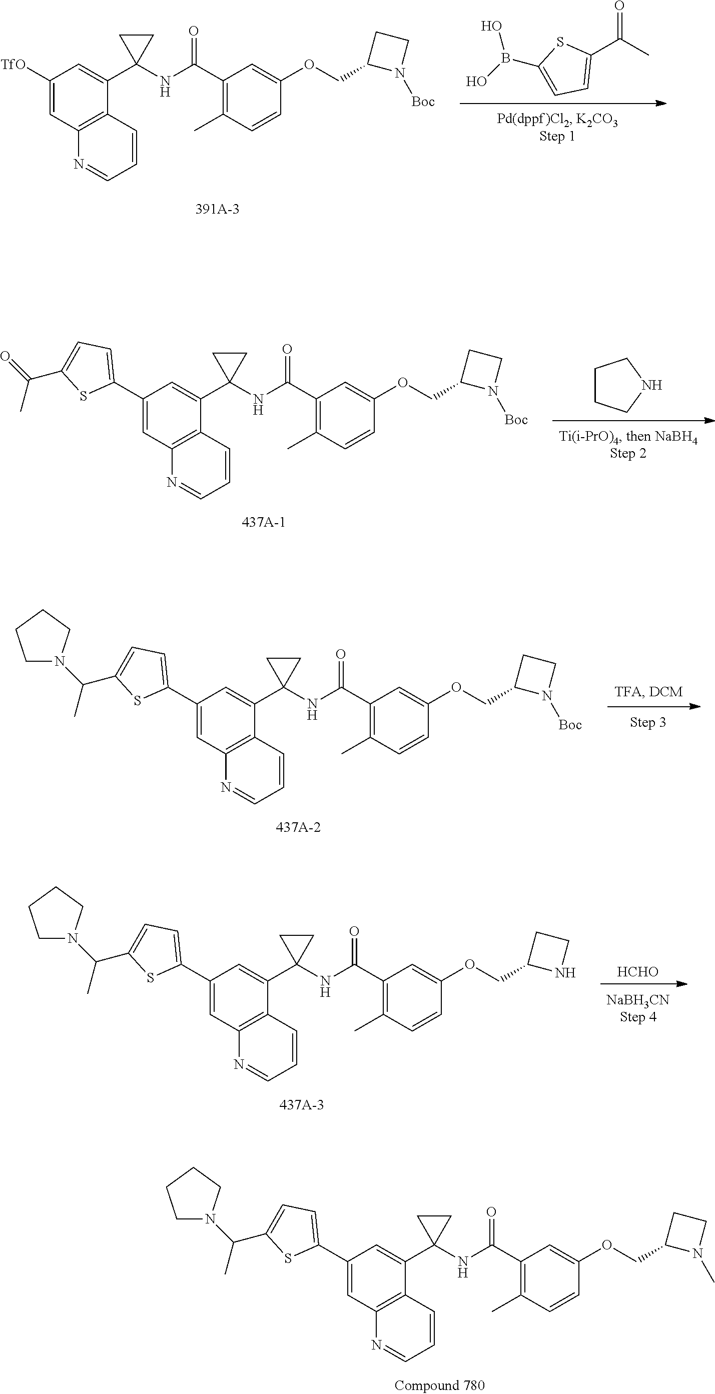

### Step 1: tert-Butyl(S)-2-((3-((1-(7-(5-acetylthiophen-2-yl)quinolin-5-yl)cyclopropyl)carbamoyl)-4-methylphenoxy)methyl)azetidine-1-carboxylate (437A-1)

A mixture of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (100 mg, 157 μmol, 1.0 eq), (5-acetyl-2-thienyl) boronic acid (53.5 mg, 314 μmol, 2.0 eq), Pd(OAc)₂ (10.6 mg, 47.2 μmol, 0.3 eq), KOAc (46.3 mg, 471 μmol, 3.0 eq) and bis(1-adamantyl)-butyl-phosphane (11.3 mg, 31.5 μmol, 0.2 eq) in DMSO (2.0 mL) was degassed and purged with N₂ three times. The resulting mixture was stirred at 80° C. under a N₂ atmosphere for 16 h. LCMS indicated that the starting material was consumed, and the desired mass was detected. The mixture was treated with water (10 mL) and extracted with DCM (10 mL×5). The combined organic layers were dried over Na₂SO₄, filtered, and concentrated under vacuum to give a residue which was purified by flash silica gel chromatography using a gradient of EtOAc/petroleum ether from 0/1 to 1/1. tert-Butyl 2-((3-(methoxycarbonyl)-4-methylphenyl) ethynyl)azetidine-1-carboxylate (130 mg) was obtained as a colorless oil. M+H⁺=612.3 (LCMS).

### Step 2: tert-Butyl(2S)-2-((4-methyl-3-((1-(7-(5-(1-(pyrrolidin-1-yl)ethyl)thiophen-2-yl)quinolin-5-yl)cyclopropyl)carbamoyl)phenoxy)methyl)azetidine-1-carboxylate (437A-2)

To a solution of tert-butyl 2-((3-(methoxycarbonyl)-4-methylphenyl) ethynyl)azetidine-1-carboxylate (130 mg, 212 μmol, 1.0 eq) in EtOH (1.0 mL) were added tetraisopropoxytitanium (603 mg, 2.13 mmol, 627 μL, 10 eq) and pyrrolidine (75.6 mg, 1.06 mmol, δ8.7 μL, 5.0 eq). The mixture was stirred at 40° C. for 12 h. The reaction mixture was allowed to cool to room temperature, then NaBH₄ (120 mg, 3.19 mmol, 15 eq) was added to the mixture. The mixture was stirred at 20° C. for 2 h. LCMS indicated that the starting material was completely consumed, and the desired mass was detected. The mixture was concentrated under vacuum at 30° C. to give a residue which was purified by preparative HPLC (Phenomenex Luna C18 column (100× 40 mm, 5 μm); flow rate: 25 mL/min; gradient: 30%-60% B over 8 min; mobile phase A: 0.1% aqueous TFA, mobile phase B: acetonitrile). tert-Butyl(2S)-2-((4-methyl-3-((1-(7-(5-(1-(pyrrolidin-1-yl)ethyl)thiophen-2-yl)quinolin-5-yl)cyclopropyl) carbamoyl)phenoxy)methyl)azetidine-1-carboxylate (20.0 mg, 22.2 μmol, 10% yield, TFA salt) was obtained as a white solid. M+H⁺=667.5 (LCMS).

### Step 3: 5-(((S)-Azetidin-2-yl)methoxy)-2-methyl-N-(1-(7-(5-(1-(pyrrolidin-1-yl)ethyl)thiophen-2-yl)quinolin-5-yl)cyclopropyl)benzamide (437A-3)

To a solution of tert-butyl(25)-2-((4-methyl-3-((1-(7-(5-(1-(pyrrolidin-1-yl)ethyl)thiophen-2-yl)quinolin-5-yl)cyclopropyl)carbamoyl)phenoxy)methyl)azetidine-1-carboxylate (20.0 mg, 29.9 μmol, 1.0 eq) in DCM (4.0 mL) was added TFA (86.6 mg, 759 μmol, 56.3 μL, 25 eq). The mixture was stirred at 20° C. for 1 h. LCMS indicated that the starting material was completely consumed, and the desired mass was detected. The reaction mixture was concentrated under vacuum at 30° C. to give the crude product 5-(((S)-azetidin-2-yl)methoxy)-2-methyl-N-(1-(7-(5-(1-(pyrrolidin-1-yl)ethyl)thiophen-2-yl)quinolin-5-yl)cyclopropyl)benzamide (20.0 mg, TFA salt) as a colorless oil. M−100+H⁺=567.3 (LCMS).

### Step 4: 2-Methyl-5-(((S)-1-methylazetidin-2-yl)methoxy)-N-(1-(7-(5-(1-(pyrrolidin-1-yl)ethyl)thiophen-2-yl)quinolin-5-yl)cyclopropyl)benzamide (Compound 780)

To a of 5-(((S)-azetidin-2-yl)methoxy)-2-methyl-N-(1-(7-(5-(1-(pyrrolidin-1-yl)ethyl)thiophen-2-yl)quinolin-5-yl)cyclopropyl)benzamide (20.0 mg, 35.3 μmol, 1.0 eq) in MeOH (2.0 mL) were added HOAc (211 μg, 3.53 μmol, 0.1 eq) and formaldehyde (4.30 mg, 52.9 μmol, 3.94 μL, 37% purity in H₂O, 1.5 eq) at 20° C. for 1 h. Then NaBH₃CN (5.54 mg, δ8.2 μmol, 2.5 eq) was added to the mixture. The mixture was stirred at 20° C. for 1 h. The mixture was concentrated under vacuum at 30° C. to give a residue which was purified by preparative HPLC (Phenomenex Luna C18 column (80×30 mm, 3 μm); flow rate: 25 mL/min; gradient: 10%-40% B over 8 min; mobile phase A: 0.04% HCl, mobile phase B: acetonitrile). 2-Methyl-5-(((S)-1-methylazetidin-2-yl)methoxy)-N-(1-(7-(5-(1-(pyrrolidin-1-yl)ethyl)thiophen-2-yl)quinolin-5-yl)cyclopropyl)benzamide (8.20 mg, 12.5 μmol, 35% yield, 2 HCl salt) was obtained as a white solid. M+H⁺=581.3 (LCMS); ¹H NMR (400 MHZ, DMSO-d₆) δ 11.55-11.40 (m, 1H), 10.89-10.73 (m, 1H), 9.51-9.28 (m, 2H), 9.13 (m, 1H), 8.43-8.24 (m, 2H), 7.93-7.82 (m, 2H), 7.56 (m, 1H), 7.14-7.06 (m, 1H), 6.96-6.87 (m, 1H), 6.74 (m, 1H), 4.96-4.84 (m, 1H), 4.71-4.57 (m, 1H), 4.38 (m, 1H), 4.21 (m, 1H), 4.03-3.94 (m, 2H), 3.90-3.84 (m, 2H), 3.00-3.07 (m, 4H), 2.80 (m, 3H), 2.67 (m, 1H), 2.37-2.25 (m, 2H), 1.96 (m, 4H), 1.78 (m, 3H), 1.50-1.30 (m, 4H).

### Example 438: (S)-2-Methyl-5-((1-methylazetidin-2-yl)methoxy)-N-(1-(7-(5-(pyrrolidin-1-ylmethyl)thiophen-2-yl)quinolin-5-yl)cyclopropyl)benzamide (Compound 669)

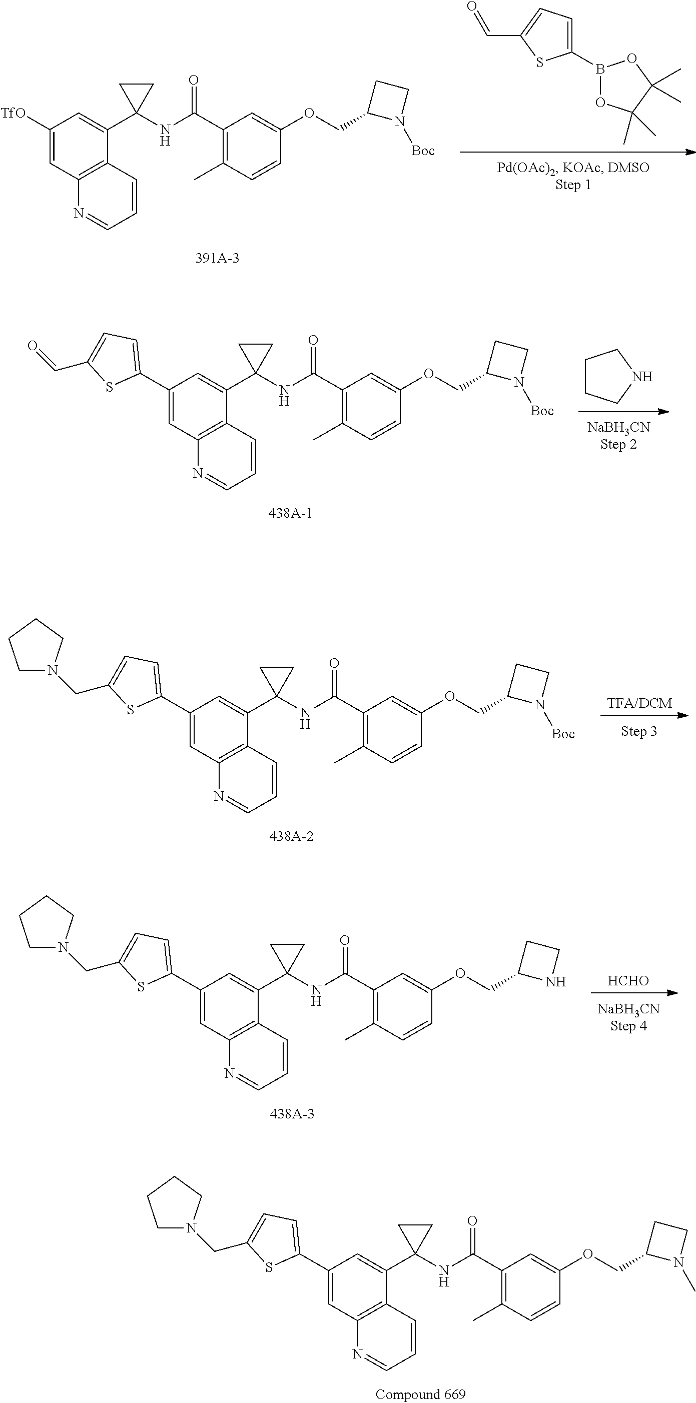

### Step 1: tert-Butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (438A-1)

A mixture of 5-(1-aminocyclopropyl)quinolin-7-yl trifluoromethanesulfonate (200 mg, 314 μmol, 1.0 eq), 5-(4,4,5,5-tetramethyl-1,3,2-dioxaborolan-2-yl)thiophene-2-carbaldehyde (58.9 mg, 377 μmol, 1.2 eq), di(1-adamantyl)-N-butylphosphine hydroiodide (22.6 mg, 63.0 μmol, 0.2 eq), Pd(OAc) 2 (7.10 mg, 31.0 μmol, 0.1 eq) and KOAc (93.0 mg, 94.0 μmol, 3.0 eq) in DMSO (3.0 mL) was degassed and purged with N₂ three times. The mixture was stirred at 80° C. for 16 h under a N₂ atmosphere. LCMS indicated that the starting material was completely consumed. The reaction was allowed to cool to room temperature. The mixture was treated with water (10 mL) and extracted with DCM (5.0 mL×2). The combined organic layers were dried over Na₂SO₄, filtered, and concentrated under vacuum to give a residue which was purified by flash silica gel chromatography using a gradient of EtOAc/petroleum ether from 0/1 to 1/1. tert-Butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (150 mg, 231 μmol, 74% yield) was obtained as a white solid. M+H⁺=598.4 (LCMS).

### Step 2: tert-Butyl(S)-2-((4-methyl-3-((1-(7-(5-(pyrrolidin-1-ylmethyl)thiophen-2-yl)quinolin-5-yl)cyclopropyl)carbamoyl)phenoxy)methyl)azetidine-1-carboxylate (438A-2)

To a solution of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (150 mg, 251 μmol, 1.0 eq) in MeOH (2.0 mL) was added HOAc (1.51 mg, 25.0 μmol, 1.44 μL, 0.1 eq), pyrrolidine (26.8 mg, 376 μmol, 31.0 μL, 1.5 eq) at 20° C. for 1 h. Then NaBH₃CN (39.4 mg, 627 μmol, 2.5 eq) was added to the mixture. The mixture was stirred at 20° C. for 1 h. LCMS indicated that the starting material was completely consumed. The mixture was treated with water (10 mL) and extracted with DCM (5.0 mL×2). The combined organic layers were dried over Na₂SO₄, filtered, and concentrated under vacuum to give a residue which was purified by flash silica gel chromatography using a gradient of EtOAc/petroleum ether from 0/1 to 0/1. tert-Butyl(S)-2-((4-methyl-3-((1-(7-(5-(pyrrolidin-1-ylmethyl)thiophen-2-yl)quinolin-5-yl)cyclopropyl)carbamoyl)phenoxy)methyl)azetidine-1-carboxylate (160 mg, 237 μmol, 95% yield) was obtained as a yellow oil. M+H⁺=653.4 (LCMS).

### Step 3: (S)-5-(Azetidin-2-ylmethoxy)-2-methyl-N-(1-(7-(5-(pyrrolidin-1-ylmethyl)thiophen-2-yl)quinolin-5-yl)cyclopropyl)benzamide (438A-3)

To a solution of tert-butyl(S)-2-((4-methyl-3-((1-(7-(5-(pyrrolidin-1-ylmethyl)thiophen-2-yl)quinolin-5-yl)cyclopropyl)carbamoyl)phenoxy)methyl)azetidine-1-carboxylate (160 mg, 245 μmol, 1.0 eq) in DCM (4.0 mL) was added TFA (708 mg, 6.21 mmol, 459 μL, 25 eq). The mixture was stirred at 20° C. for 1 h. LCMS indicated that the starting material was completely consumed, and the desired mass was detected. The reaction mixture was concentrated under vacuum at 30° C. to give the crude product(S)-5-(azetidin-2-ylmethoxy)-2-methyl-N-(1-(7-(5-(pyrrolidin-1-ylmethyl)thiophen-2-yl)quinolin-5-yl)cyclopropyl)benzamide (170 mg, TFA salt) as a yellow oil, which was used in the next step without any further purification. M+H⁺=553.4 (LCMS).

### Step 4: (S)-2-Methyl-5-((1-methylazetidin-2-yl)methoxy)-N-(1-(7-(5-(pyrrolidin-1-ylmethyl)thiophen-2-yl)quinolin-5-yl)cyclopropyl)benzamide (Compound 669)

To a solution of(S)-5-(azetidin-2-ylmethoxy)-2-methyl-N-(1-(7-(5-(pyrrolidin-1-ylmethyl)thiophen-2-yl)quinolin-5-yl)cyclopropyl)benzamide (170 mg, 307 μmol, 1.0 eq) in MeOH (2.0 mL) were added HOAc (1.85 mg, 30.0 μmol, 1.76 μL, 0.1 eq) and formaldehyde (37.4 mg, 461 μmol, 34.4 μL, 37% purity in H₂O, 1.5 eq) at 20° C. for 1 h. Then NaBH₃CN (48.3 mg, 768 μmol, 2.5 eq) was added to the mixture. The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Gemini C18 column (80×30 mm, 3 μm); flow rate: 25 mL/min; gradient: 1%-25% B over 8 min; mobile phase A: 0.04% aqueous HCl, mobile phase B: acetonitrile). (S)-2-Methyl-5-((1-methylazetidin-2-yl)methoxy)-N-(1-(7-(5-(pyrrolidin-1-ylmethyl)thiophen-2-yl)quinolin-5-yl)cyclopropyl)benzamide (64.5 mg, 94.8 μmol, 31% yield, 2 HCl salt) was obtained as a yellow solid. M+H⁺=567.4 (LCMS); 1H NMR (400 MHZ, DMSO-d₆) δ 11.26-11.11 (m, 1H), 11.07-10.87 (m, 1H), 9.60-9.41 (m, 1H), 9.36 (m, 1H), 9.16 (m, 1H), 8.40 (m, 1H), 8.32 (m, 1H), 7.94-7.88 (m, 1H), 7.87 (m, 1H), 7.54 (m, 1H), 7.09 (m, 1H), 6.92 (m, 1H), 6.75 (m, 1H), 4.66 (m, 3H), 4.40 (m, 1H), 4.25-4.18 (m, 1H), 4.05-3.93 (m, 1H), 3.89-3.77 (m, 1H), 3.50-3.38 (m, 2H), 3.18-3.06 (m, 2H), 2.80 (m, 3H), 2.37-2.24 (m, 2H), 2.03 (m, 2H), 1.97 (m, 3H), 1.94-1.87 (m, 2H), 1.48-1.43 (m, 2H), 1.40-1.35 (m, 2H).

### Example 439: (S)-2-Methyl-5-((1-methylazetidin-2-yl)methoxy)-N-(1-(7-(5-(pyrrolidine-1-carbonyl)thiophen-2-yl)quinolin-5-yl)cyclopropyl)benzamide (Compound 767)

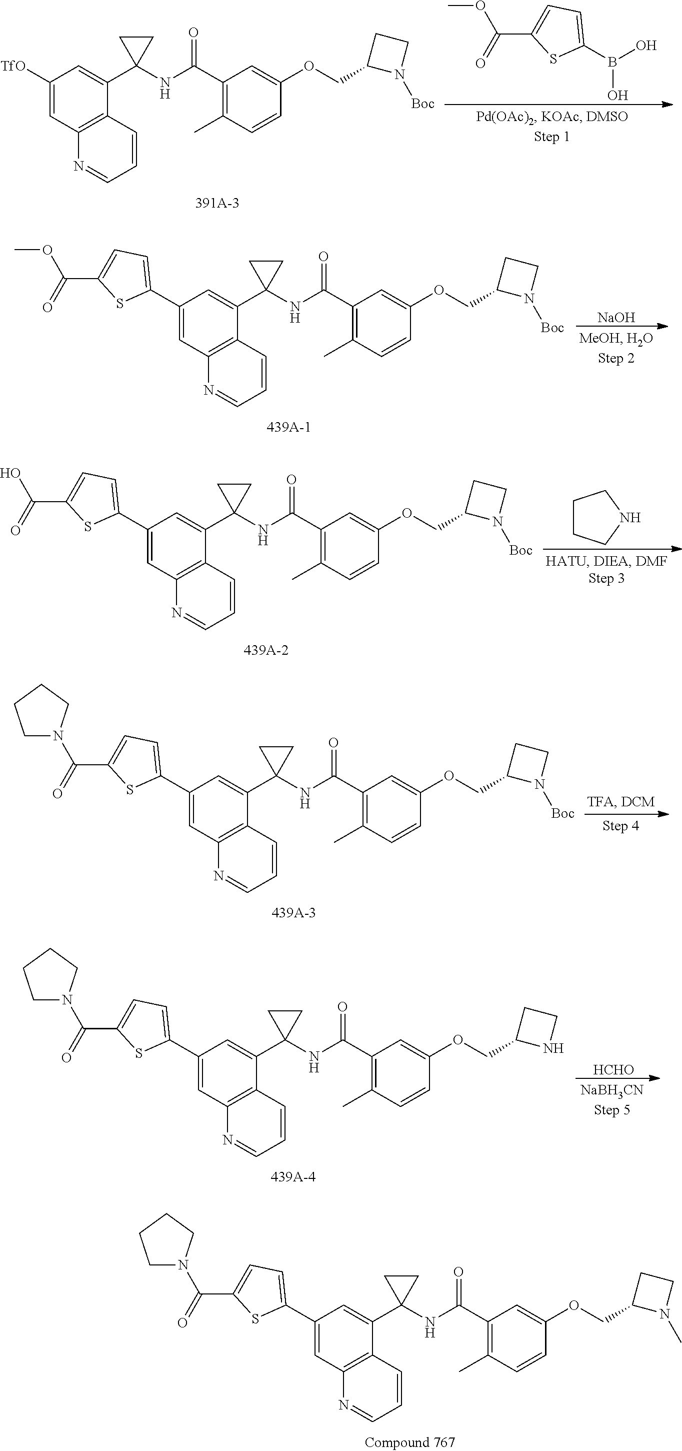

### Step 1: tert-Butyl(S)-2-((3-((1-(7-(5-(methoxycarbonyl)thiophen-2-yl)quinolin-5-yl)cyclopropyl)carbamoyl)-4-methylphenoxy)methyl)azetidine-1-carboxylate (439A-1)

To a solution of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (120 mg, 188 μmol, 1.0 eq) and (5-(methoxycarbonyl)thiophen-2-yl) boronic acid (70.2 mg, 377 μmol, 2.0 eq) in DMSO (2.0 mL) were added Pd(OAc)₂ (12.7 mg, 56.6 μmol, 0.30 eq), KOAc (55.6 mg, 566 μmol, 3.0 eq) and bis(1-adamantyl)-butyl-phosphane (13.5 mg, 37.7 μmol, 0.2 eq) at 20° C. and then the mixture was stirred at 80° C. for 12 h under a N₂ atmosphere. LCMS indicated that the starting material was completely consumed. The mixture was allowed to cool to room temperature, treated with water (10 mL), and extracted with DCM (10 mL×2). The combined organic layers were dried over Na₂SO₄, filtered, and concentrated under vacuum to give a residue which was purified by flash silica gel chromatography using a gradient of EtOAc/petroleum ether 0/1 from to tert-Butyl(S)-2-((3-((1-(7-(5-1/1.(methoxycarbonyl)thiophen-2-yl)quinolin-5-yl)cyclopropyl)carbamoyl)-4-methylphenoxy)methyl)azetidine-1-carboxylate (140 mg) was obtained as a yellow liquid. M+H⁺=628.3 (LCMS).

### Step 2: (S)-5-(5-(1-(5-((1-(tert-Butoxycarbonyl)azetidin-2-yl)methoxy)-2-methylbenzamido)cyclopropyl)quinolin-7-yl)thiophene-2-carboxylic acid (439A-2)

To a solution of tert-butyl(S)-2-((3-((1-(7-(5-(methoxycarbonyl)thiophen-2-yl)quinolin-5-yl)cyclopropyl)carbamoyl)-4-methylphenoxy)methyl)azetidine-1-carboxylate (120 mg, 191 μmol, 1.0 eq) in a mixture of H₂O (1.0 mL), THF (1.0 mL) and MeOH (1.0 mL) was added LiOH·H₂O (80.2 mg, 1.91 mmol, 10 eq). The mixture was stirred at 20° C. for 2 h. LCMS indicated that the starting material was completely consumed. The reaction mixture was concentrated under vacuum to give a residue, which was diluted with H₂O (5.0 mL). The aqueous layer was adjusted to pH 6 with HCl (1 M aqueous), then extracted with EtOAc (5.0 mL×3). The combined organic layers were dried over Na₂SO₄, filtered, and concentrated under vacuum to give the crude product(S)-5-(5-(1-(5-((1-(tert-butoxycarbonyl)azetidin-2-yl)methoxy)-2-methylbenzamido)cyclopropyl)quinolin-7-yl)thiophene-2-carboxylic acid (92.0 mg) as a white solid. M+H⁺=614.3 (LCMS).

### Step 3: tert-Butyl(S)-2-((4-methyl-3-((1-(7-(5-(pyrrolidine-1-carbonyl)thiophen-2-yl)quinolin-5-yl)cyclopropyl)carbamoyl)phenoxy)methyl)azetidine-1-carboxylate (439A-3)

To a solution of(S)-5-(5-(1-(5-((1-(tert-butoxycarbonyl)azetidin-2-yl)methoxy)-2-methylbenzamido)cyclopropyl)quinolin-7-yl)thiophene-2-carboxylic acid (92.0 mg, 149 μmol, 1.0 eq) in DMF (5.0 mL) were added HATU (114 mg, 299 μmol, 2.0 eq) and DIEA (58.1 mg, 449 μmol, 78.33 μL, 3.0 eq). The reaction mixture was stirred at 20° C. for 20 min. Then pyrrolidine (10.6 mg, 149 μmol, 12.5 μL, 1.0 eq) was added and the mixture was stirred at 20° C. for 2 h. LCMS indicated that the starting material was completely consumed. The reaction mixture was poured into H₂O (5.0 mL) and extracted with EtOAc (5.0 mL×3). The combined organic layers were dried over Na₂SO₄, filtered, and concentrated under vacuum to give tert-butyl(S)-2-((4-methyl-3-((1-(7-(5-(pyrrolidine-1-carbonyl)thiophen-2-yl)quinolin-5-yl)cyclopropyl)carbamoyl)phenoxy)methyl)azetidine-1-carboxylate (99.6 mg 149 μmol, 99% yield) as a brown gum. M+H⁺=667.4 (LCMS).

### Step 4: (S)-5-(Azetidin-2-ylmethoxy)-2-methyl-N-(1-(7-(5-(pyrrolidine-1-carbonyl)thiophen-2-yl)quinolin-5-yl)cyclopropyl)benzamide (439A-4)

To a solution of tert-butyl(S)-2-((4-methyl-3-((1-(7-(5-(pyrrolidine-1-carbonyl)thiophen-2-yl)quinolin-5-yl)cyclopropyl)carbamoyl)phenoxy)methyl)azetidine-1-carboxylate (99.6 mg, 149 μmol, 1.0 eq) in DCM (2.0 mL) was added TFA (2.0 mL). The mixture was stirred at 20° C. for 1 h. LCMS indicated that the starting material was completely consumed. The reaction mixture was concentrated under vacuum at 30° C. to give the crude product(S)-5-(azetidin-2-ylmethoxy)-2-methyl-N-(1-(7-(5-(pyrrolidine-1-carbonyl)thiophen-2-yl)quinolin-5-yl)cyclopropyl)benzamide (70.0 mg) as a yellow gum. M+H⁺=567.3 (LCMS).

### Step 5: (S)-2-Methyl-5-((1-methylazetidin-2-yl)methoxy)-N-(1-(7-(5-(pyrrolidine-1-carbonyl)thiophen-2-yl)quinolin-5-yl)cyclopropyl)benzamide (Compound 767)

To a solution of(S)-5-(azetidin-2-ylmethoxy)-2-methyl-N-(1-(7-(5-(pyrrolidine-1-carbonyl)thiophen-2-yl)quinolin-5-yl)cyclopropyl)benzamide (70.0 mg, 123 μmol, 1.0 eq) in MeOH (2.0 mL) were added HOAc (1.85 mg, 30.0 μmol, 1.76 μL, 0.1 eq) and formaldehyde (20.0 mg, 247 μmol, 18.3 μL, 37% purity in H₂O, 2.0 eq) at 20° C. The mixture was stirred at 20° C. for 1 h, then NaBH₃CN (48.3 mg, 768 μmol, 2.5 eq) was added. The mixture was stirred at 20° C. for 1 h. LCMS indicated that the starting material was completely consumed. The mixture was concentrated under vacuum to give a residue which was purified by preparative HPLC (Phenomenex Luna (80×30 mm, 3 μm); flow rate: 25 mL/min; gradient: gradient: 10%-40% B over 8 min; mobile phase A: 0.04% HCl, mobile phase B: acetonitrile). (S)-2-Methyl-5-((1-methylazetidin-2-yl)methoxy)-N-(1-(7-(5-(pyrrolidine-1-carbonyl)thiophen-2-yl)quinolin-5-yl)cyclopropyl)benzamide (42.5 mg, 63.7 μmol, 51% yield, HCl salt) was obtained as a yellow solid. M+H⁺=581.4 (LCMS); ¹H NMR (400 MHz, DMSO-d₆) δ 9.22 (s, 1H), 9.15 (br d, J=8.9 Hz, 1H), 9.00 (br d, J=3.0 Hz, 1H), 8.27 (s, 1H), 8.22 (d, J=1.6 Hz, 1H), 7.82 (d, J=3.9 Hz, 1H), 7.75-7.63 (m, 2H), 7.10 (d, J=8.6 Hz, 1H), 6.98-6.87 (m, 1H), 6.72 (d, J=2.9 Hz, 1H), 4.66-4.51 (m, 1H), 4.31-4.18 (m, 2H), 4.06-3.95 (m, 1H), 3.90-3.77 (m, 4H), 2.82 (d, J=5.0 Hz, 3H), 2.72-2.63 (m, 1H), 2.42-2.23 (m, 2H), 1.95 (s, 5H), 1.91-1.83 (m, 2H), 1.46-1.26 (m, 4H).

### Example 440: (S)—N-(1-(7-(4-Chloro-2-methylthiazol-5-yl)quinolin-5-yl)cyclopropyl)-2-methyl-5-((1-methylazetidin-2-yl)methoxy)benzamide (Compound 812)

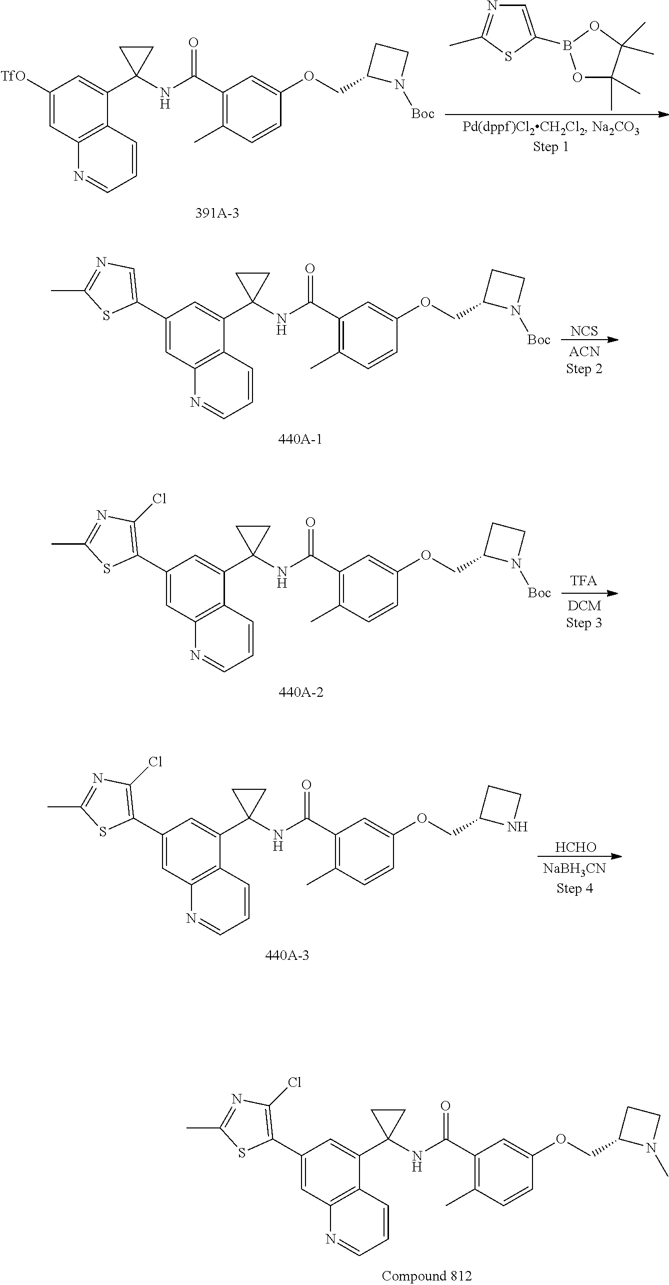

### Step 1: tert-Butyl(S)-2-((4-methyl-3-((1-(7-(2-methylthiazol-5-yl)quinolin-5-yl)cyclopropyl)carbamoyl)phenoxy)methyl)azetidine-1-carboxylate (440A-1)

To a stirred solution of tert-butyl(S)-2-((4-methyl-3-((1-(7-(((trifluoromethyl) sulfonyl)oxy)quinolin-5-yl)cyclopropyl)carbamoyl)phenoxy)methyl)azetidine-1-carboxylate (200 mg, 315 μmol, 1.0 eq) and 2-methyl-5-(4,4,5,5-tetramethyl-1,3,2-dioxaborolan-2-yl) thiazole (106 mg, 472 μmol, 1.5 eq) in a mixture of dioxane (8.0 mL) and H₂O (0.8 mL) were added Na₂CO₃ (76.7 mg, 724 μmol, 2.3 eq) and Pd(dppf)Cl₂·CH₂Cl₂ (30.8 mg, 37.8 μmol, 0.12 eq) in one portion. The mixture was degassed and purged with N₂ three times and then stirred at 80° C. for 16 h under a N₂ atmosphere. LCMS indicated that the starting material was completely consumed, and the desired mass was detected. The reaction mixture was allowed to cool to room temperature, poured into H₂O (20 mL) and extracted with EtOAc (20 mL×4). The combined organic layers were dried over Na₂SO₄, filtered, and concentrated under vacuum to give a residue which was purified by flash silica gel chromatography using a gradient of EtOAc/petroleum ether from 0/1 to 1/1. tert-Butyl(S)-2-((4-methyl-3-((1-(7-(2-methylthiazol-5-yl)quinolin-5-yl)cyclopropyl)carbamoyl)phenoxy)methyl)azetidine-1-carboxylate (260 mg, 444 μmol, 71% yield) was obtained as a yellow solid. M+H⁺=585.4 (LCMS); ¹H NMR (400 MHz, CDCl₃) δ 9.25-9.15 (m, 1H), 8.99-8.91 (m, 1H), 8.21 (br d, J=14.6 Hz, 2H), 8.07 (s, 1H), 7.58-7.49 (m, 1H), 7.02 (s, 1H), 6.85 (s, 1H), 6.77 (br s, 1H), 4.47-4.36 (m, 1H), 4.24-4.17 (m, 1H), 4.05-3.99 (m, 1H), 3.90-3.81 (m, 2H), 2.78 (s, 3H), 2.35-2.22 (m, 2H), 2.17 (s, 3H), 1.32 (br s, 13H).

### Step 2: tert-Butyl(S)-2-((3-((1-(7-(4-chloro-2-methylthiazol-5-yl)quinolin-5-yl)cyclopropyl)carbamoyl)-4-methylphenoxy)methyl)azetidine-1-carboxylate (440A-2)

To a mixture of tert-butyl(S)-2-((4-methyl-3-((1-(7-(2-methylthiazol-5-yl)quinolin-5-yl)cyclopropyl)carbamoyl)phenoxy)methyl)azetidine-1-carboxylate (210 mg, 360 μmol, 1.0 eq) in acetonitrile (9.0 mL) was added NCS (57.0 mg, 432 μmol, 1.2 eq). The mixture was stirred at 60° C. for 16 h under a N₂ atmosphere. LCMS indicated that the starting material was completely consumed, and the desired mass was detected. The reaction mixture was allowed to cool to room temperature, poured into H₂O (10 mL) and extracted with EtOAc (20 mL×4). The combined organic layers were dried over Na₂SO₄, filtered, and concentrated under vacuum to give a residue which was purified by preparative TLC (DCM/EtOAc=2/1, R_{f}=0.4). tert-Butyl(S)-2-((3-((1-(7-(4-chloro-2-methylthiazol-5-yl)quinolin-5-yl)cyclopropyl)carbamoyl)-4-methylphenoxy)methyl)azetidine-1-carboxylate (40.0 mg, 64.6 μmol, 18% yield) was obtained as a white solid. M+H⁺=619.4 (LCMS); ¹H NMR (400 MHZ, CDCl₃) δ 9.19-9.11 (m, 1H), 8.97 (br d, J=2.9 Hz, 1H), 8.35 (s, 1H), 8.25 (d, J=1.5 Hz, 1H), 7.58-7.50 (m, 1H), 7.05-7.01 (m, 1H), 6.86-6.82 (m, 1H), 6.80-6.75 (m, 1H), 4.49-4.38 (m, 1H), 4.25-4.17 (m, 1H), 4.06-3.98 (m, 1H), 3.92-3.82 (m, 2H), 2.75 (s, 3H), 2.35-2.20 (m, 2H), 2.16 (s, 3H), 1.33 (br s, 13H).

### Step 3: (S)-5-(Azetidin-2-ylmethoxy)-N-(1-(7-(4-chloro-2-methylthiazol-5-yl)quinolin-5-yl)cyclopropyl)-2-methylbenzamide (440A-3)

To a solution of tert-butyl(S)-2-((3-((1-(7-(4-chloro-2-methylthiazol-5-yl)quinolin-5-yl)cyclopropyl)carbamoyl)-4-methylphenoxy)methyl)azetidine-1-carboxylate (80.0 mg, 130 μmol, 1.0 eq) in DCM (2.0 mL) was added TFA (221 mg, 1.04 mmol, 142 μL, 15 eq). The mixture was stirred at 20° C. for 1 h. LCMS indicated that the starting material was completely consumed, and the desired mass was detected. The mixture was concentrated under vacuum at 20° C. to give(S)-5-(azetidin-2-ylmethoxy)-N-(1-(7-(4-chloro-2-methylthiazol-5-yl)quinolin-5-yl)cyclopropyl)-2-methylbenzamide (80.0 mg, TFA salt) as a yellow oil, which was used in the next step without any further purification. M+H⁺=519.3 (LCMS).

### Step 4: (S)—N-(1-(7-(4-Chloro-2-methylthiazol-5-yl)quinolin-5-yl)cyclopropyl)-2-methyl-5-((1-methylazetidin-2-yl)methoxy)benzamide (Compound 812)

To a solution of(S)-5-(azetidin-2-ylmethoxy)-N-(1-(7-(4-chloro-2-methylthiazol-5-yl)quinolin-5-yl)cyclopropyl)-2-methylbenzamide (80.0 mg, 126 μmol, 1.0 eq, TFA salt) in MeOH (2.0 mL) was added TEA (100 μL), followed by formaldehyde (7.69 mg, 94.8 μmol, 7.06 μL, 37% purity in water, 1.5 eq). The resulting mixture was adjusted to pH 6 with a small amount of AcOH. The mixture was stirred at 20° C. for 30 min, then NaBH₃CN (7.94 mg, 126 μmol, 2.0 eq) was added. The resulting mixture was stirred at 20° C. for another 1 h. LCMS indicated that the starting material was completely consumed, and the desired mass was detected. The reaction mixture was poured into H₂O (10 mL) and extracted with DCM (10 mL×5). The combined organic layers were dried over Na₂SO₄, filtered, and concentrated under vacuum to give a residue which was purified by preparative HPLC (Phenomenex Gemini C18 column (80×30 mm, 3 μm); flow rate: 25 mL/min; gradient: 15%-45% B over 8 min; mobile phase A: 0.04% aqueous HCl, mobile phase B: acetonitrile). (S)—N-(1-(7-(4-Chloro-2-methylthiazol-5-yl)quinolin-5-yl)cyclopropyl)-2-methyl-5-((1-methylazetidin-2-yl)methoxy)benzamide (30.0 mg, 51.4 μmol, 41% yield, HCl salt) was obtained as a yellow solid. M+H⁺=533.2 (LCMS); ¹H NMR (400 MHZ, DMSO-d₆) δ 9.33 (br d, J=8.1 Hz, 1H), 9.26 (s, 1H), 9.10 (d, J=3.4 Hz, 1H), 8.34 (d, J=0.9 Hz, 1H), 8.21 (d, J=1.5 Hz, 1H), 7.84 (dd, J=4.6, 8.6 Hz, 1H), 7.10 (d, J=8.4 Hz, 1H), 6.92 (dd, J=2.7, 8.4 Hz, 1H), 6.73 (d, J=2.6 Hz, 1H), 4.61 (br dd, J=3.0, 7.3 Hz, 1H), 4.34-4.18 (m, 2H), 4.01 (dt, J=4.5, 9.5 Hz, 1H), 3.86 (q, J=9.7 Hz, 1H), 2.82 (s, 3H), 2.75-2.69 (m, 3H), 2.43-2.25 (m, 2H), 1.97 (s, 3H), 1.43 (br s, 2H), 1.30 (br s, 2H).

### Example 441: (S)—N-(1-(8-Chloro-7-(2-methylthiazol-5-yl)quinolin-5-yl)cyclopropyl)-2-methyl-5-((1-methylazetidin-2-yl)methoxy)benzamide (Compound 814)

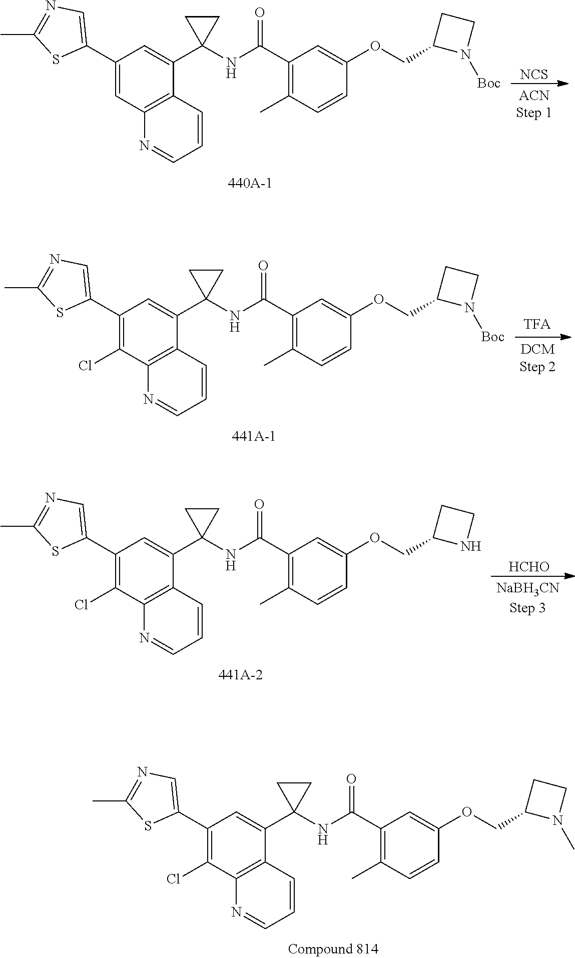

### Step 1: tert-Butyl(S)-2-((3-((1-(8-chloro-7-(2-methylthiazol-5-yl)quinolin-5-yl)cyclopropyl)carbamoyl)-4-methylphenoxy)methyl)azetidine-1-carboxylate (441A-1)

To a mixture of tert-butyl(S)-2-((4-methyl-3-((1-(7-(2-methylthiazol-5-yl)quinolin-5-yl)cyclopropyl)carbamoyl)phenoxy)methyl)azetidine-1-carboxylate (210 mg, 360 μmol, 1.0 eq) in acetonitrile (9.0 mL) was added NCS (57.0 mg, 432 μmol, 1.2 eq). The mixture was stirred at 60° C. for 16 h under a N₂ atmosphere. LCMS indicated that the starting material was completely consumed, and the desired mass was detected. The reaction mixture was allowed to cool to room temperature, poured into H₂O (10 mL), and extracted with EtOAc (20 mL×4). The combined organic layers were dried over Na₂SO₄, filtered, and concentrated under vacuum to give a residue which was purified by preparative TLC (DCM/EtOAc=2/1, R_{f}=0.4). tert-Butyl(S)-2-((3-((1-(8-chloro-7-(2-methylthiazol-5-yl)quinolin-5-yl)cyclopropyl)carbamoyl)-4-methylphenoxy)methyl)azetidine-1-carboxylate (100 mg, 161 μmol, 45% yield) was obtained as a yellow solid. M+H⁺=619.3 (LCMS); ¹H NMR (400 MHZ, CDCl₃) δ 9.21-9.15 (m, 1H), 9.13-9.07 (m, 1H), 8.15 (s, 1H), 8.08 (s, 1H), 7.63-7.53 (m, 1H), 7.06-6.98 (m, 1H), 6.86-6.82 (m, 1H), 6.77-6.74 (m, 1H), 4.47-4.38 (m, 1H), 4.25-4.16 (m, 1H), 4.05-3.99 (m, 1H), 3.91-3.82 (m, 2H), 2.80 (s, 3H), 2.16 (s, 3H), 1.45 (br s, 13H).

### Step 2: (S)-5-(Azetidin-2-ylmethoxy)-N-(1-(8-chloro-7-(2-methylthiazol-5-yl)quinolin-5-yl)cyclopropyl)-2-methylbenzamide (441A-2)

To a solution of tert-butyl(S)-2-((3-((1-(8-chloro-7-(2-methylthiazol-5-yl)quinolin-5-yl)cyclopropyl)carbamoyl)-4-methylphenoxy)methyl)azetidine-1-carboxylate (140 mg, 226 μmol, 1.0 eq) in DCM (4.0 mL) was added TFA (251 μL). The mixture was stirred at 20° C. for 1 h. LCMS indicated that the starting material was completely consumed, and the desired mass was detected. The mixture was concentrated under vacuum at 20° C. to give(S)-5-(azetidin-2-ylmethoxy)-N-(1-(8-chloro-7-(2-methylthiazol-5-yl)quinolin-5-yl)cyclopropyl)-2-methylbenzamide (140 mg, TFA salt) as a yellow oil, which was used in the next step without any further purification. M+H⁺=519.2 (LCMS).

### Step 3: (S)—N-(1-(8-Chloro-7-(2-methylthiazol-5-yl)quinolin-5-yl)cyclopropyl)-2-methyl-5-((1-methylazetidin-2-yl)methoxy)benzamide (Compound 814)

To a solution of(S)-5-(azetidin-2-ylmethoxy)-N-(1-(8-chloro-7-(2-methylthiazol-5-yl)quinolin-5-yl)cyclopropyl)-2-methylbenzamide (140 mg, 221 μmol, 1.0 eq, TFA salt) in MeOH (4.0 mL) was added TEA (200 μL), followed by formaldehyde (27.0 mg, 332 μmol, 24.9 μL, 37% purity in water, 1.5 eq). The resulting mixture was adjusted to pH 6 with a small amount of AcOH. The mixture was stirred at 20° C. for 30 min, then NaBH₃CN (28.0 mg, 442 μmol, 2.0 eq) was added. The reaction mixture was stirred at 20° C. for 1 h. LCMS indicated that the starting material was completely consumed, and the desired mass was detected. The reaction mixture was poured into H₂O (10 mL) and extracted with DCM (20 mL×5). The combined organic layers were dried over Na₂SO₄, filtered, and concentrated under vacuum to give a residue which was purified by preparative HPLC (Phenomenex Gemini C18 column (80× 30 mm, 3 μm); flow rate: 25 mL/min; gradient: 15%-45% B over 8 min; mobile phase A: 0.04% aqueous HCl, mobile phase B: acetonitrile). (S)—N-(1-(8-Chloro-7-(2-methylthiazol-5-yl)quinolin-5-yl)cyclopropyl)-2-methyl-5-((1-methylazetidin-2-yl)methoxy)benzamide (46.4 mg, 81.5 μmol, 37% yield, HCl salt) was obtained as a green solid. M+H⁺=533.2 (LCMS); ¹H NMR (400 MHZ, DMSO-d₆) δ 9.27-9.18 (m, 1H), 9.12-9.02 (m, 1H), 8.30 (s, 1H), 8.13 (s, 1H), 7.78 (dd, J=4.2, 8.5 Hz, 1H), 7.08 (d, J=8.6 Hz, 1H), 6.91 (dd, J=2.6, 8.4 Hz, 1H), 6.72 (d, J=2.7 Hz, 1H), 4.63 (dq, J=2.9, 8.5 Hz, 1H), 4.36 (br dd, J=8.0, 11.3 Hz, 1H), 4.20 (br dd, J=3.1, 11.2 Hz, 1H), 3.99 (td, J=4.8, 9.5 Hz, 1H), 3.89-3.85 (m, 1H), 2.82-2.66 (m, 6H), 2.39-2.25 (m, 2H), 1.96 (s, 3H), 1.40 (br s, 2H), 1.34 (br s, 2H).

### Example 442: (S)-2-Methyl-5-((1-(methyl-d₃)azetidin-2-yl)methoxy)-N-(1-(7-(2-methyloxazol-5-yl)quinolin-5-yl)cyclopropyl)benzamide (Compound 782)

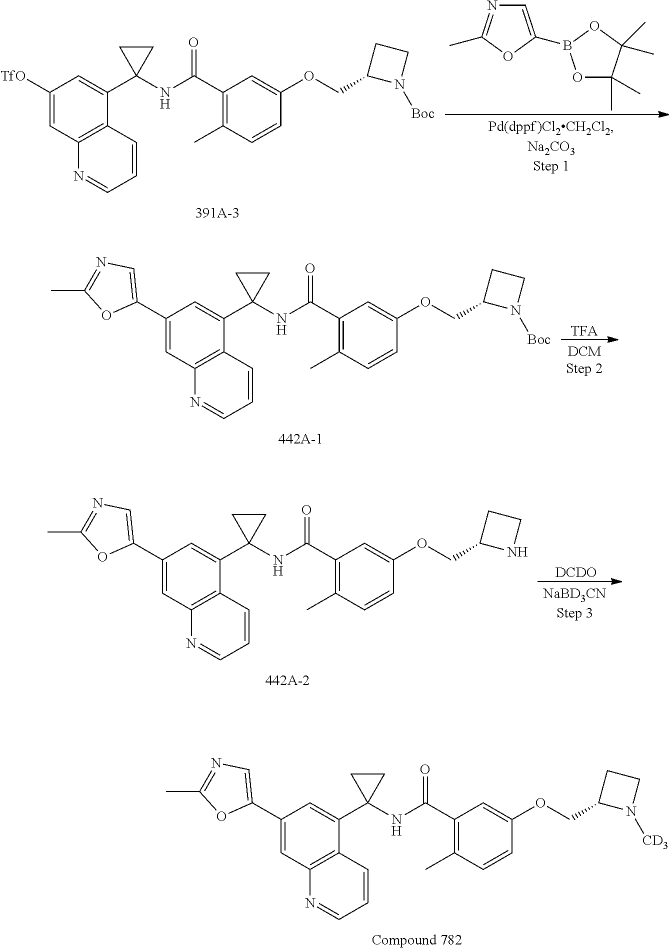

### Step 1: tert-Butyl(S)-2-((4-methyl-3-((1-(7-(2-methyloxazol-5-yl)quinolin-5-yl)cyclopropyl)carbamoyl)phenoxy)methyl)azetidine-1-carboxylate (442A-1)

To a stirred solution of tert-butyl(S)-2-((4-methyl-3-((1-(7-(((trifluoromethyl)sulfonyl)oxy) quinolin-5-yl)cyclopropyl)carbamoyl)phenoxy)methyl)azetidine-1-carboxylate (150 mg, 236 μmol, 1.0 eq) and 2-methyl-5-(4,4,5,5-tetramethyl-1,3,2-dioxaborolan-2-yl)oxazole (98.7 mg, 472 μmol, 2.0 eq) in a mixture of dioxane (5.0 mL) and H₂O (0.5 mL) were added Na₂CO₃ (57.5 mg, 543 μmol, 2.3 eq) and Pd(dppf)Cl₂·CH₂Cl₂ (28.9 mg, 35.4 μmol, 0.2 eq). The mixture was degassed and purged with N₂ three times and then stirred at 80° C. for 16 h under a N₂ atmosphere. LCMS indicated that the starting material was completely consumed, and the desired mass was detected. The reaction mixture was allowed to cool to room temperature, poured into H₂O (20 mL) and extracted with EtOAc (20 mL×4). The combined organic layers were dried over Na₂SO₄, filtered, and concentrated under vacuum to give a residue which was purified by preparative TLC (EtOAc/petroleum ether=1/0, R_{f}=0.3). tert-Butyl(S)-2-((4-methyl-3-((1-(7-(2-methyloxazol-5-yl)quinolin-5-yl)cyclopropyl)carbamoyl)phenoxy)methyl)azetidine-1-carboxylate (110 mg, 193 μmol, 82% yield) was obtained as a yellow solid. M+H⁺=569.4 (LCMS); ¹H NMR (400 MHZ, CDCl₃) δ 9.29-9.11 (m, 1H), 9.01-8.89 (m, 1H), 8.41-8.31 (m, 1H), 8.27-8.20 (m, 1H), 7.57-7.48 (m, 2H), 7.07-7.01 (m, 1H), 6.88-6.72 (m, 3H), 4.46-4.37 (m, 1H), 4.26-4.17 (m, 1H), 4.06-4.00 (m, 1H), 3.91-3.82 (m, 2H), 2.62-2.58 (m, 3H), 2.33-2.21 (m, 2H), 2.19-2.14 (m, 3H), 1.32 (br s, 13H).

### Step 2: (S)-5-(Azetidin-2-ylmethoxy)-2-methyl-N-(1-(7-(2-methyloxazol-5-yl)quinolin-5-yl)cyclopropyl)benzamide (442A-2)

To a solution of tert-butyl(S)-2-((4-methyl-3-((1-(7-(2-methyloxazol-5-yl)quinolin-5-yl)cyclopropyl)carbamoyl)phenoxy)methyl)azetidine-1-carboxylate (110 mg, 193 μmol, 1.0 eq) in DCM (2.0 mL) was added TFA (441 mg, 3.87 mmol, 286 μL, 20 eq). The mixture was stirred at 20° C. for 1 h. LCMS indicated that the starting material was completely consumed, and the desired mass was detected. The mixture was concentrated under vacuum at 30° C. to give the crude(S)-5-(azetidin-2-ylmethoxy)-2-methyl-N-(1-(7-(2-methyloxazol-5-yl)quinolin-5-yl)cyclopropyl)benzamide (110 mg) as a yellow oil, which was used in the next step without any further purification. M+H⁺=469.3 (LCMS).

### Step 3: (S)-2-Methyl-5-((1-(methyl-d₃)azetidin-2-yl)methoxy)-N-(1-(7-(2-methyloxazol-5-yl)quinolin-5-yl)cyclopropyl)benzamide (Compound 782)

To a solution of(S)-5-(azetidin-2-ylmethoxy)-2-methyl-N-(1-(7-(2-methyloxazol-5-yl)quinolin-5-yl)cyclopropyl)benzamide (110 mg, 189 μmol, 11.7 μL, 1.0 eq, TFA salt) in MeOH (2.0 mL) was added TEA (100 μL), followed by formaldehyde-d₂ (35.2 mg, 227 μmol, 4.73 μL, 20% purity in D₂O, 1.2 eq). The resulting mixture was adjusted to pH 6 with a small amount of AcOH. The mixture was stirred at 20° C. for 30 min, then NaBD₃CN (11.9 mg, 189 μmol, 1.0 eq) was added. The reaction mixture was stirred at 20° C. for another 1 h. LCMS indicated that the starting material was completely consumed, and the desired mass was detected. The reaction mixture was poured into H₂O (10 mL) and extracted with DCM (10 mL×5). The combined organic layers were dried over Na₂SO₄, filtered, and concentrated under vacuum to give a residue which was purified by preparative HPLC (Phenomenex Gemini C18 column (80×30 mm, 3 μm); flow rate: 25 mL/min; gradient: 1%-30% B over 8 min; mobile phase A: 0.04% aqueous HCl, mobile phase B: acetonitrile). (S)-2-Methyl-5-((1-(methyl-d₃)azetidin-2-yl)methoxy)-N-(1-(7-(2-methyloxazol-5-yl)quinolin-5-yl)cyclopropyl)benzamide (52.1 mg, 96.6 μmol, 51% yield, HCl salt) was obtained as a yellow solid. M+H⁺=486.2 (LCMS); ¹H NMR (400 MHZ, DMSO-d₆) δ 9.56-9.49 (m, 1H), 9.36-9.31 (m, 1H), 9.22-9.15 (m, 1H), 8.34-8.29 (m, 2H), 8.00-7.93 (m, 2H), 7.13-7.06 (m, 1H), 6.98 (s, 1H), 6.80-6.71 (m, 1H), 4.68-4.59 (m, 1H), 4.35-4.28 (m, 1H), 4.26-4.19 (m, 1H), 4.06-3.96 (m, 1H), 3.91-3.81 (m, 1H), 2.60-2.57 (m, 3H), 2.41 (br s, 2H), 1.99-1.94 (m, 3H), 1.44 (br s, 2H), 1.41-1.35 (m, 2H).

### Example 443: (S)—N-(1-(7-(2-Acetyloxazol-5-yl)quinolin-5-yl)cyclopropyl)-2-methyl-5-((1-methylazetidin-2-yl)methoxy)benzamide (Compound 806)

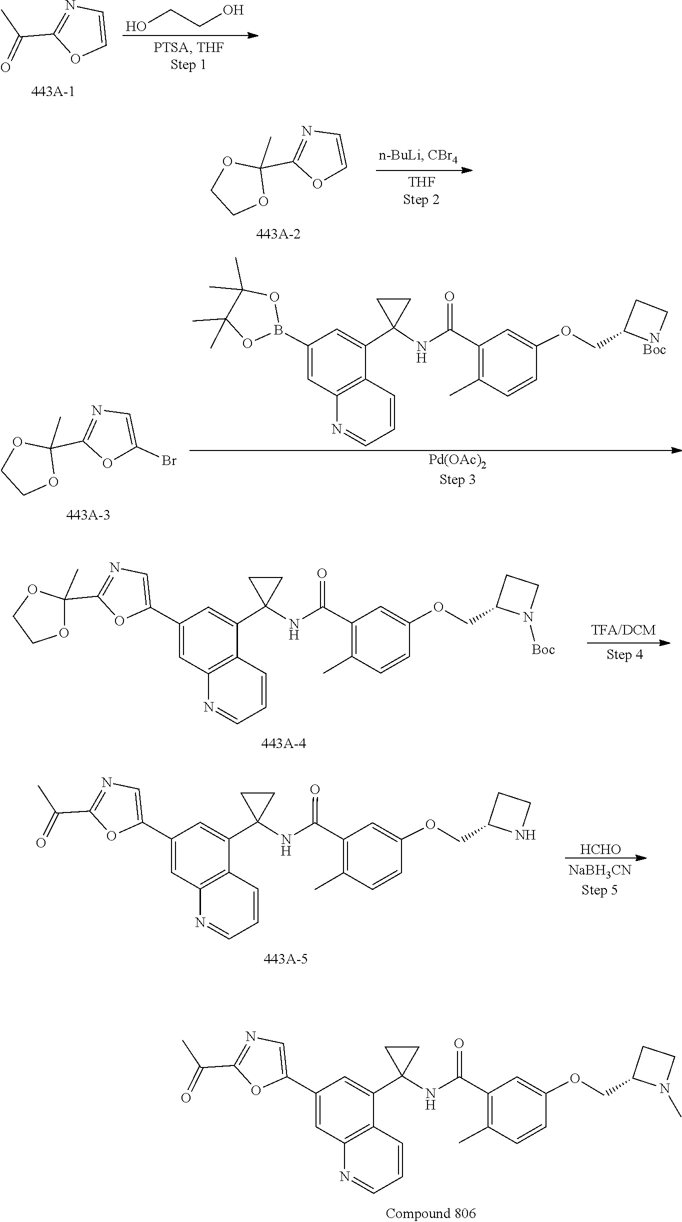

### Step 1: 2-(2-Methyl-1,3-dioxolan-2-yl)oxazole (443A-2)

To a solution of 1-(oxazol-2-yl) ethan-1-one (300 mg, 2.70 mmol, 1.0 eq) in toluene (3.0 mL) was added PTSA (23.3 mg, 135 μmol, 0.05 eq) at 20° C. for 1 h. Then ethylene glycol (3.35 g, 54.0 mmol, 3.0 mL, 20 eq) was added and the mixture was stirred at 130° C. for 12 h under a N₂ atmosphere. LCMS indicated that the starting material was completely consumed. The mixture was allowed to cool to room temperature and concentrated under vacuum to give a residue which was purified by preparative HPLC (Phenomenex Gemini C18 column (100× 30 mm, 5 μm); flow rate: 25 mL/min; gradient: 5%-40% B over 8 min; mobile phase A: 0.1% aqueous TFA, mobile phase B: acetonitrile). 2-(2-Methyl-1,3-dioxolan-2-yl)oxazole (200 mg, 1.25 mmol, 46% yield) was obtained as a yellow oil. M+H⁺=156 (LCMS).

### Step 2: 5-Bromo-2-(2-methyl-1,3-dioxolan-2-yl)oxazole (443A-3)

To a solution of 2-(2-methyl-1,3-dioxolan-2-yl)oxazole (100 mg, 644 μmol, 1.0 eq) in THF (3.0 mL) was added n-BuLi (2.5 M, 644 μL, 2.5 eq) at −78° C., the mixture was stirred at −78° C. for 1 h. Then carbon tetrabromide (384 mg, 1.16 mmol, 1.8 eq) was added to the mixture at −78° C., and then the mixture was allowed to warm up to 20° C. and stirred at 20° C. for 2 h. LCMS indicated that the starting material was completely consumed. The mixture was poured into water (10 mL) and extracted with DCM (5.0 mL×2). The combined organic layers were dried over Na₂SO₄, filtered, and concentrated under vacuum to give a residue which was purified by flash silica gel chromatography using a gradient of EtOAc/petroleum ether from 0/1 to 1/3. 5-Bromo-2-(2-methyl-1,3-dioxolan-2-yl)oxazole (110 mg, 455 μmol, 70% yield) was obtained as a yellow oil. M+H⁺=234.2/236.2 (LCMS).

### Step 3: tert-Butyl(S)-2-((4-methyl-3-((1-(7-(2-(2-methyl-1,3-dioxolan-2-yl)oxazol-5-yl)quinolin-5-yl)cyclopropyl)carbamoyl)phenoxy)methyl)azetidine-1-carboxylate (443A-4)

A mixture of tert-butyl(25)-2-[4-methyl-3-[1-[7-(4,4,5,5-tetramethyl-1,3,2-dioxaborolan-2-yl)-5-quinolyl]cyclopropyl]carbamoyl]phenoxy]methyl]azetidine-1-carboxylate (196 mg, 320 μmol, 1.0 eq), 5-bromo-2-(2-methyl-1,3-dioxolan-2-yl)oxazole (90.0 mg, 384 μmol, 1.2 eq), Pd(dppf)Cl₂ (23.5 mg, 32.0 μmol, 0.10 eq), and Na₂CO₃ (101 mg, 961 μmol, 3.0 eq) in a mixture of dioxane (1.0 mL) and H₂O (200 μL) was degassed and purged with N₂ three times. The resulting mixture was stirred at 80° C. under a N₂ atmosphere for 16 h. LCMS indicated that the starting material was completely consumed. The mixture was allowed to cool to room temperature, treated with water (10 mL), and extracted with DCM (5.0 mL×2). The combined organic layers were dried over Na₂SO₄, filtered, and concentrated under vacuum to give a residue which was purified by flash silica gel chromatography using a gradient of EtOAc/petroleum ether from 0/1 to 1/0. tert-Butyl(S)-2-((4-methyl-3-((1-(7-(2-(2-methyl-1,3-dioxolan-2-yl)oxazol-5-yl)quinolin-5-yl)cyclopropyl)carbamoyl)phenoxy)methyl)azetidine-1-carboxylate (90.0 mg, 122 μmol, 38% yield) was obtained as a white amorphous solid. M+H⁺=641.6 (LCMS).

### Step 4: (S)—N-(1-(7-(2-Acetyloxazol-5-yl)quinolin-5-yl)cyclopropyl)-5-(azetidin-2-ylmethoxy)-2-methylbenzamide (443A-5)

To a solution of tert-butyl(S)-2-((4-methyl-3-((1-(7-(2-(2-methyl-1,3-dioxolan-2-yl)oxazol-5-yl)quinolin-5-yl)cyclopropyl)carbamoyl)phenoxy)methyl)azetidine-1-carboxylate (90.0 mg, 140 μmol, 1.0 eq) in a mixture of DCM (2.0 mL) and H₂O (1.0 mL) was added TFA (16.0 mg, 140 μmol, 10.4 μL, 1.0 eq). The mixture was stirred at 20° C. for 60 h. LCMS indicated that the starting material was completely consumed, and the desired mass was detected. The reaction mixture was concentrated under vacuum at 30° C. to give the crude product(S)—N-(1-(7-(2-acetyloxazol-5-yl)quinolin-5-yl)cyclopropyl)-5-(azetidin-2-ylmethoxy)-2-methylbenzamide (80.0 mg, 128 μmol, TFA salt) as a yellow solid. M+H⁺=497.3 (LCMS).

### Step 5: (S)—N-(1-(7-(2-Acetyloxazol-5-yl)quinolin-5-yl)cyclopropyl)-2-methyl-5-((1-methylazetidin-2-yl)methoxy)benzamide (Compound 806)

To a solution of(S)—N-(1-(7-(2-acetyloxazol-5-yl)quinolin-5-yl)cyclopropyl)-5-(azetidin-2-ylmethoxy)-2-methylbenzamide (70 mg, 114 μmol, 1.0 eq, TFA salt) in MeOH (2.0 mL) were added HOAc (688 μg, 11.4 μmol, 0.1 eq) and formaldehyde (13.9 mg, 171 μmol, 12.8 μL, 37% purity in H₂O, 1.5 eq) at 20° C. for 1 h. Then NaBH₃CN (7.20 mg, 114 μmol, 1.0 eq) was added to the mixture. The mixture was stirred at 20° C. for 1 h. LCMS indicated that the starting material was completely consumed. The mixture was concentrated under vacuum to give a residue which was purified by preparative HPLC (Waters Xbridge Prep OBD C18 column (80×30 mm, 3 μm); flow rate: 25 mL/min; gradient: 5%-35% B over 8 min; mobile phase A: 0.04% aqueous HCl, mobile phase B: acetonitrile). (S)—N-(1-(7-(2-Acetyloxazol-5-yl)quinolin-5-yl)cyclopropyl)-2-methyl-5-((1-methylazetidin-2-yl)methoxy)benzamide (4.00 mg, 7.31 μmol, 6% yield) was obtained as a white solid. M+H⁺=511.4 (LCMS); ¹H NMR (400 MHZ, CDCl₃) δ 10.92-10.77 (m, 1H), 9.46-9.32 (m, 2H), 9.21-9.09 (m, 1H), 8.50 (m, 1H), 8.42-8.30 (m, 2H), 7.95-7.80 (m, 1H), 7.15-7.03 (m, 1H), 6.95-6.88 (m, 1H), 6.75 (m, 1H), 4.71-4.56 (m, 1H), 4.39 (m, 1H), 4.26-4.18 (m, 1H), 4.04-3.93 (m, 1H), 3.92-3.81 (m, 2H), 2.80 (m, 3H), 2.72-2.61 (m, 3H), 2.39-2.23 (m, 2H), 1.96 (m, 3H), 1.53-1.31 (m, 3H).

### Example 444: (S)—N-(1-(7-(1H-Pyrazol-1-yl)quinolin-5-yl)cyclopropyl)-2-methyl-5-((1-methylazetidin-2-yl)methoxy)benzamide (Compound 619)

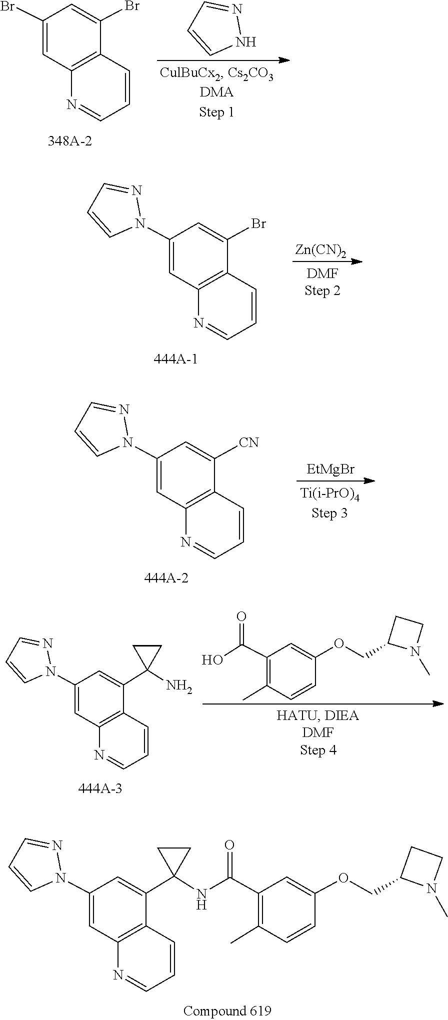

### Step 1: 5-Bromo-7-(1H-pyrazol-1-yl)quinoline (444A-1)

A solution of 5,7-dibromoquinoline (2.50 g, 8.71 mmol, 1.0 eq) in DMA (175 mL) was degassed and purged with N₂ three times. To the mixture were added 1H-pyrazole (711 mg, 10.4 mmol, 1.2 eq), CuiBuCx₂ (347 mg, 871 μmol, 0.1 eq) and Cs₂CO₃ (5.68 g, 17.4 mmol, 2.0 eq) at 20° C. The resulting mixture was stirred at 120° C. for 14 h under a N₂ atmosphere. LCMS indicated that the starting material was completely consumed, and the desired mass was detected. The reaction mixture was allowed to cool to room temperature, poured into H₂O (200 mL), and extracted with EtOAc (100 mL×3). The combined organic layers were dried over Na₂SO₄, filtered, and concentrated under vacuum to give a residue which was purified by preparative TLC (EtOAc/petroleum ether=1/1, R_{f}=0.3). 5-Bromo-7-(1H-pyrazol-1-yl)quinoline (400 mg, 1.46 mmol, 8% yield) was obtained as a colorless oil. M+H⁺=273.9 (LCMS); ¹H NMR (400 MHZ, CDCl₃) δ 8.99-8.93 (m, 1H), 8.57-8.50 (m, 2H), 8.23 (d, J=1.6 Hz, 1H), 8.13 (d, J=2.5 Hz, 1H), 7.82 (s, 1H), 7.51 (dd, J=4.3, 8.5 Hz, 1H), 6.57 (t, J=2.1 Hz, 1H).

### Step 2: 7-(1H-Pyrazol-1-yl)quinoline-5-carbonitrile (444A-2)

A solution of 5-bromo-7-(1H-pyrazol-1-yl)quinoline (900 mg, 3.28 mmol, 1.0 eq) in DMF (90 mL) was degassed and purged with N₂ three times. To the mixture were added Zn(CN)₂ (771 mg, 6.57 mmol, 2.0 eq), Brettphos Pd G3 (595 mg, 657 μmol, 0.2 eq) and Brettphos (353 mg, 657 μmol, 0.2 eq) at 20° C. The resulting mixture was stirred at 80° C. for 1 h under a N₂ atmosphere. TLC indicated that the starting material was completely consumed. The reaction mixture was allowed to cool to room temperature, poured into H₂O (100 mL), and extracted with EtOAc (50 mL×3). The combined organic layers were dried over Na₂SO₄, filtered, and concentrated under vacuum to give a residue which was purified by flash silica gel chromatography using a gradient of EtOAc/petroleum ether from 1/100 to 1/1. 7-(1H-Pyrazol-1-yl)quinoline-5-carbonitrile (200 mg, 908 mmol, 28% yield) was obtained as a white solid. ¹H NMR (400 MHZ, CDCl₃) δ 9.08 (dd, J=1.5, 4.3 Hz, 1H), 8.66 (d, J=2.3 Hz, 1H), 8.56 (d, J=8.4 Hz, 1H), 8.49 (d, J=2.0 Hz, 1H), 8.15 (d, J=2.5 Hz, 1H), 7.84 (d, J=1.5 Hz, 1H), 7.62 (dd, J=4.3, 8.5 Hz, 1H), 6.66-6.58 (m, 1H).

### Step 3: 1-(7-(1H-Pyrazol-1-yl)quinolin-5-yl)cyclopropan-1-amine (444A-3)

A mixture of 7-(1H-pyrazol-1-yl)quinoline-5-carbonitrile (50.0 mg, 227 μmol, 1.0 eq) in anhydrous Et₂O (5.0 mL) was degassed and purged with N₂ three times, then cooled to −78° C. To this mixture was added Ti(i-PrO)₄ (71.0 mg, 250 μmol, 73.7 μL, 1.1 eq) slowly, and then EtMgBr (3 M in Et₂O, 166 μL, 2.2 eq) was added dropwise to maintain the temperature between −78° C. and −75° C. over 1 h under a N₂ atmosphere. After the addition was complete, the resulting mixture was stirred at the same temperature for 10 min and warmed to 20° C. over 1 h. BF₃·Et₂O (64.5 mg, 454 μmol, 48.3 mL, 2.0 eq) was added slowly with no obvious temperature change. LCMS indicated that the starting material was completely consumed, and the desired mass was detected. The reaction mixture was added into a mixture of HCl (1 M aqueous) (5.0 mL) and MTBE (5.0 mL) and extracted with MTBE (5.0 mL×2). The aqueous layer was basified to pH 8 by using NaOH (2 M aqueous), and a precipitate was formed. The mixture was filtered through a pad of Celite and the slurry was washed with DCM several times. The combined filtrate was extracted with DCM (5.0 mL×2). The combined organic layers were dried over Na₂SO₄, filtered, and concentrated under vacuum to give a residue which was purified by preparative TLC (DCM/MeOH=1/1, R_{f}=0.6). 1-(7-(1H-Pyrazol-1-yl)quinolin-5-yl)cyclopropan-1-amine (50.0 mg, 200 μmol, 22% yield) was obtained as a yellow oil. M+H⁺=251.1 (LCMS).

### Step 4: (S)—N-(1-(7-(1H-Pyrazol-1-yl)quinolin-5-yl)cyclopropyl)-2-methyl-5-((1-methylazetidin-2-yl)methoxy)benzamide (Compound 619)

To a solution of 1-(7-(1H-pyrazol-1-yl)quinolin-5-yl)cyclopropan-1-amine (40.0 mg, 160 μmol, 3.0 eq) and(S)-2-methyl-5-((1-methylazetidin-2-yl)methoxy)benzoic acid (12.5 mg, 53.3 μmol, 1.0 eq) in DMF (4 mL) were added HATU (50.6 mg, 133 μmol, 2.5 eq) and DIEA (20.6 mg, 160 μmol, 27.8 μL, 3 eq). The mixture was stirred at 20° C. for 4 h. LCMS indicated that the starting material was completely consumed, and the desired mass was detected. The reaction mixture was poured into H₂O (5.0 mL) and extracted with DCM (3.0 mL×2). The combined organic layers were dried over Na₂SO₄, filtered, and concentrated under vacuum to give a residue which was purified by preparative HPLC (Waters Xbridge BEH C18 column (100×30 mm, 10 μm); flow rate: 25 mL/min; gradient: 1%-30% B over 8 min; mobile phase A: 0.04% aqueous HCl, mobile phase B: acetonitrile). (S)—N-(1-(7-(1H-Pyrazol-1-yl)quinolin-5-yl)cyclopropyl)-2-methyl-5-((1-methylazetidin-2-yl)methoxy)benzamide (4.5 mg, 9.42 μmol, 18% yield) was obtained as a yellow solid. M+H⁺=468.1 (LCMS); ¹H NMR (400 MHz, CD₃OD) δ 9.86-9.77 (m, 1H), 9.19 (d, J=4.9 Hz, 1H), 8.78 (s, 1H), 8.69 (d, J=2.0 Hz, 1H), 8.52 (s, 1H), 8.11-8.00 (m, 1H), 7.92 (d, J=1.5 Hz, 1H), 7.18-7.10 (m, 1H), 6.98 (dd, J=2.6, 8.3 Hz, 1H), 6.85 (d, J=2.8 Hz, 1H), 6.75-6.68 (m, 1H), 4.72-4.66 (m, 1H), 4.35-4.14 (m, 3H), 4.02-3.90 (m, 1H), 2.95 (s, 3H), 2.60-2.51 (m, 2H), 2.04 (s, 3H), 1.63 (br s, 2H), 1.58-1.52 (m, 2H).

### Example 445: (S)-2-Methyl-5-((1-methylazetidin-2-yl)methoxy)-N-(1-(7-(pyridin-3-yl)quinolin-5-yl)cyclopropyl)benzamide (Compound 653)

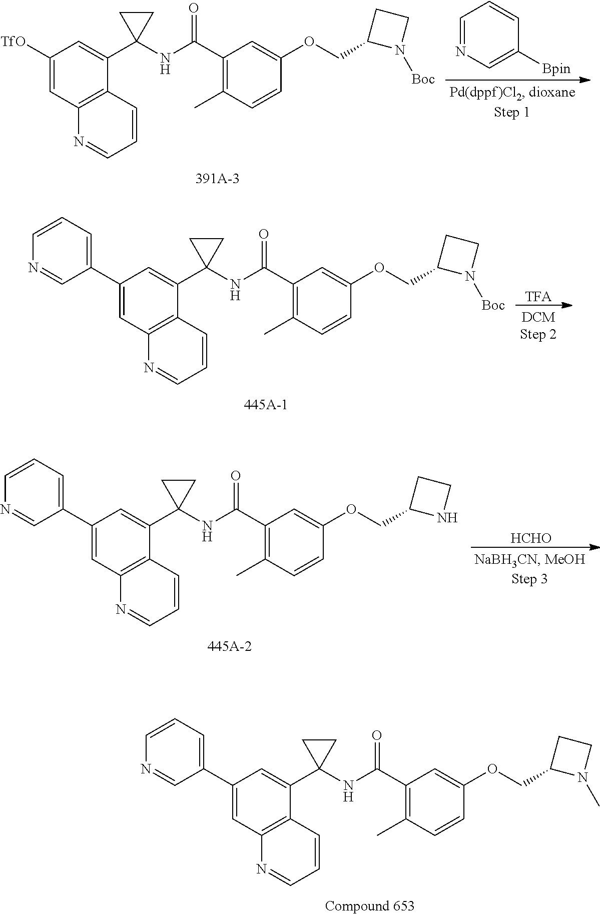

### Step 1: tert-Butyl(S)-2-((4-methyl-3-((1-(7-(pyridin-3-yl)quinolin-5-yl)cyclopropyl)carbamoyl)phenoxy)methyl)azetidine-1-carboxylate (445A-1)

To a solution of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (200 mg, 315 μmol, 1.0 eq) and 3-(4,4,5,5-tetramethyl-1,3,2-dioxaborolan-2-yl)pyridine (64.5 mg, 315 μmol, 1.0 eq) in a mixture of dioxane (20 mL) and H₂O (6.0 mL) were added Pd(dppf)Cl₂·CH₂Cl₂ (25.7 mg, 31.5 μmol, 0.1 eq) and Na₂CO₃ (76.7 mg, 724 μmol, 2.3 eq). The mixture was stirred at 80° C. for 16 h under a N₂ atmosphere. LCMS indicated that the starting material was completely consumed, and the desired mass was detected. The reaction mixture was allowed to cool to room temperature, poured into H₂O (15 mL), and extracted with EtOAc (6.0 mL×3). The combined organic layers were dried over Na₂SO₄, filtered, and concentrated under vacuum to give a residue which was purified by flash silica gel chromatography using a gradient of EtOAc/petroleum ether from 1/100 to 4/5. tert-Butyl(S)-2-((4-methyl-3-((1-(7-(pyridin-3-yl)quinolin-5-yl)cyclopropyl)carbamoyl)phenoxy)methyl)azetidine-1-carboxylate (140 mg, 248 μmol, 79% yield) was obtained as a white solid. M+H⁺=565.5 (LCMS).

### Step 2: (S)-5-(Azetidin-2-ylmethoxy)-2-methyl-N-(1-(7-(pyridin-3-yl)quinolin-5-yl)cyclopropyl)benzamide (445A-2)

To a solution of tert-butyl(S)-2-((4-methyl-3-((1-(7-(pyridin-3-yl)quinolin-5-yl)cyclopropyl) carbamoyl)phenoxy)methyl)azetidine-1-carboxylate (140 mg, 248 μmol, 1.0 eq) in DCM (7.0 mL) was added TFA (2.32 g, 20.4 mmol, 1.5 mL, 82 eq). The mixture was stirred at 20° C. for 1 h. LCMS indicated that the starting material was completely consumed, and the desired mass was detected. The reaction mixture was concentrated under vacuum to give the crude(S)-5-(azetidin-2-ylmethoxy)-2-methyl-N-(1-(7-(pyridin-3-yl)quinolin-5-yl)cyclopropyl)benzamide (130 mg, TFA salt) as a yellow oil. M+H⁺=465.2 (LCMS).

### Step 3: (S)-2-Methyl-5-((1-methylazetidin-2-yl)methoxy)-N-(1-(7-(pyridin-3-yl)quinolin-5-yl)cyclopropyl)benzamide (Compound 653)

To a solution of(S)-5-(azetidin-2-ylmethoxy)-2-methyl-N-(1-(7-(pyridin-3-yl)quinolin-5-yl)cyclopropyl)benzamide (120 mg, 207 μmol, 1.0 eq, TFA salt) in MeOH (10 mL) was added TEA (1.0 mL), followed by formaldehyde (415 mg, 415 μmol, 38.1 μL, 37% purity in water, 2.0 eq). The resulting mixture was treated with a small amount of AcOH to adjust the pH 6, then NaBH₃CN (26.1 mg, 415 μmol, 2.0 eq) was added. The mixture was stirred at 20° C. for 16 h. LCMS indicated that the starting material was completely consumed, and the desired mass was detected. The reaction mixture was poured into H₂O (10 mL) and extracted with EtOAc (10 mL×3). The combined organic layers were dried over Na₂SO₄, filtered, and concentrated under vacuum to give a residue which was purified by preparative HPLC (Phenomenex C18 (75×30 mm, 3 μm); flow rate: 25 mL/min; gradient: 1%-25% B over 8 min; mobile phase A: 0.04% aqueous HCl, mobile phase B: acetonitrile). (S)-2-Methyl-5-((1-methylazetidin-2-yl)methoxy)-N-(1-(7-(pyridin-3-yl)quinolin-5-yl)cyclopropyl)benzamide (36.6 mg, 61.5 μmol, 30% yield, HCl salt) was obtained as a yellow solid. M+H⁺=479.2 (LCMS); ¹H NMR (400 MHZ, DMSO-d₆) δ 9.27-9.13 (m, 3H), 9.03 (dd, J=1.5, 4.1 Hz, 1H), 8.75 (dd, J=1.4, 5.0 Hz, 1H), 8.50 (br d, J=8.0 Hz, 1H), 8.36 (d, J=1.4 Hz, 1H), 8.26 (d, J=1.8 Hz, 1H), 7.82-7.62 (m, 2H), 7.10 (d, J=8.5 Hz, 1H), 6.92 (dd, J=2.8, 8.4 Hz, 1H), 6.72 (d, J=2.6 Hz, 1H), 4.68-4.54 (m, 1H), 4.51-4.16 (m, 4H), 2.83 (d, J=4.6 Hz, 3H), 2.38-2.30 (m, 2H), 1.97 (s, 3H), 1.41 (s, 4H).

### Example 446: (S)-5-(Azetidin-2-ylmethoxy)-2-methyl-N-(1-(7-(5-morpholinopyridin-3-yl)quinolin-5-yl)cyclopropyl)benzamide (Compound 689)

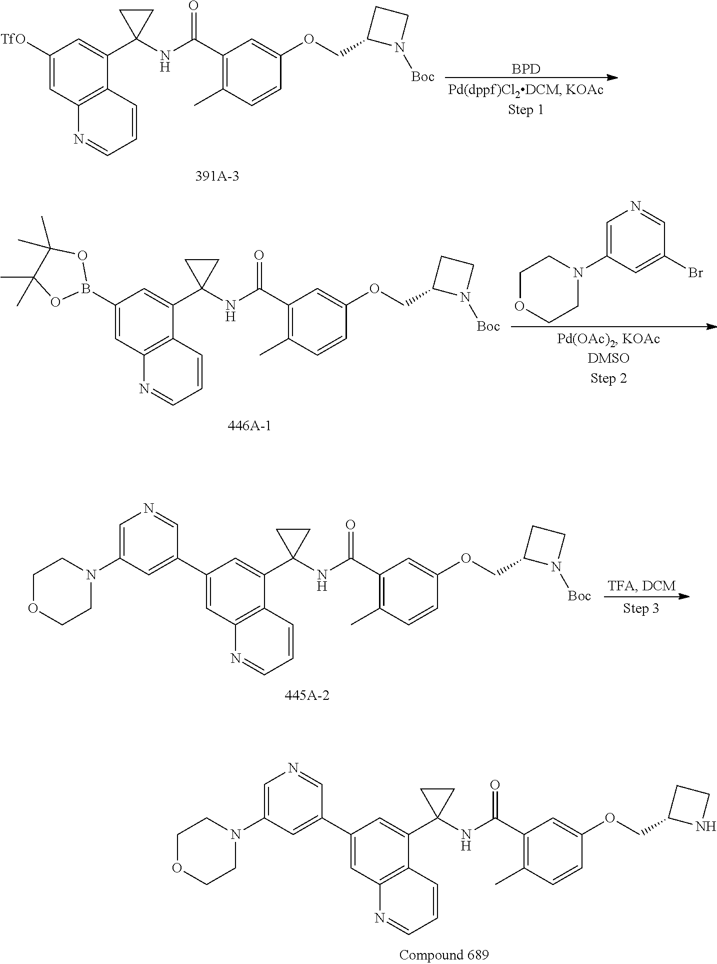

### Step 1: tert-Butyl(S)-2-((4-methyl-3-((1-(7-(4,4,5,5-tetramethyl-1,3,2-dioxaborolan-2-yl)quinolin-5-yl)cyclopropyl)carbamoyl)phenoxy)methyl)azetidine-1-carboxylate (446A-1)

A mixture of tert-butyl(S)-2-((4-methyl-3-((1-(7-(((trifluoromethyl)sulfonyl)oxy)quinolin-5-yl)cyclopropyl)carbamoyl)phenoxy)methyl)azetidine-1-carboxylate (800 mg, 1.26 mmol, 1.0 eq), BPD (639 mg, 2.52 mmol, 2.0 eq), KOAc (308 mg, 3.15 mmol, 2.5 eq), Pd(dppf)Cl₂·CH₂Cl₂ (102 mg, 126 μmol, 0.10 eq) in dioxane (20 mL) was degassed and purged with N₂ three times. The resulting mixture was stirred at 80° C. for 16 h under a N₂ atmosphere. LCMS indicated that the starting material was completely consumed. The mixture was allowed to cool to room temperature, treated with water (10 mL), and extracted with DCM (10 mL×2). The combined organic layers were dried over Na₂SO₄, filtered, and concentrated under vacuum to give a residue which was purified by flash silica gel chromatography using a gradient of EtOAc/petroleum ether from 0/1 to 2/1. tert-Butyl(S)-2-((4-methyl-3-((1-(7-(4,4,5,5-tetramethyl-1,3,2-dioxaborolan-2-yl)quinolin-5-yl)cyclopropyl)carbamoyl)phenoxy)methyl)azetidine-1-carboxylate (420 mg, 376 μmol, 30% yield) was obtained as a white solid. M−82+H⁺=532.4 (LCMS).

### Step 2: tert-Butyl(S)-2-((4-methyl-3-((1-(7-(5-morpholinopyridin-3-yl)quinolin-5-yl)cyclopropyl)carbamoyl)phenoxy)methyl)azetidine-1-carboxylate (446A-2)

A mixture of tert-butyl(S)-2-((4-methyl-3-((1-(7-(4,4,5,5-tetramethyl-1,3,2-dioxaborolan-2-yl)quinolin-5-yl)cyclopropyl)carbamoyl)phenoxy)methyl)azetidine-1-carboxylate (150 mg, 246 μmol, 1.0 eq), 4-(5-bromo-3-pyridyl) morpholine (65.8 mg, 270 μmol, 1.1 eq), Pd(OAc) 2 (16.6 mg, 73.8 μmol, 0.3 eq), bis(1-adamantyl)-butyl-phosphane (17.6 mg, 49.2 μmol, 0.2 eq) and KOAc (72.5 mg, 738 μmol, 3.0 eq) in DMSO (2.0 mL) was degassed and purged with N₂ three times. The resulting mixture was stirred at 80° C. for 16 h under a N₂ atmosphere. LCMS indicated that the starting material was completely consumed. The mixture was allowed to cool to room temperature, treated with water (10 mL) and extracted with DCM (10 mL×2). The combined organic layers were dried over Na₂SO₄, filtered, and concentrated under vacuum to give a residue which was purified by flash silica gel chromatography using a gradient of EtOAc/petroleum ether from 0/1 to 2/1. tert-Butyl(S)-2-((4-methyl-3-((1-(7-(5-morpholinopyridin-3-yl)quinolin-5-yl)cyclopropyl)carbamoyl)phenoxy)methyl)azetidine-1-carboxylate (170 mg, 143 μmol, 58% yield) was obtained as a white solid. M+H⁺=650.5 (LCMS).

### Step 3: (S)-5-(Azetidin-2-ylmethoxy)-2-methyl-N-(1-(7-(5-morpholinopyridin-3-yl)quinolin-5-yl)cyclopropyl)benzamide (Compound 689)

To a solution of tert-butyl(S)-2-((4-methyl-3-((1-(7-(5-morpholinopyridin-3-yl)quinolin-5-yl)cyclopropyl)carbamoyl)phenoxy)methyl)azetidine-1-carboxylate (170 mg, 261 μmol, 1.0 eq) in DCM (2.0 mL) was added TFA (755 mg, 6.63 mmol, 490 μL, 25 eq). The mixture was stirred at 20° C. for 1 h. LCMS indicated that the starting material was completely consumed, and the desired mass was detected. The mixture was concentrated under vacuum at 30° C. to give a residue which was purified by preparative HPLC (Phenomenex Gemini C18 column (100×30 mm, 5 μm); flow rate: 25 mL/min; gradient: 1%-28% B over 8 min; mobile phase A: 0.1% aqueous TFA, mobile phase B: acetonitrile). (S)-5-(Azetidin-2-ylmethoxy)-2-methyl-N-(1-(7-(5-morpholinopyridin-3-yl)quinolin-5-yl)cyclopropyl)benzamide (22.5 mg, 33.9 μmol, 13% yield, TFA salt) was obtained as a yellow solid. M+H⁺=550.3 (LCMS); ¹H NMR (400 MHZ, DMSO-d₆) δ 9.21 (m, 1H), 9.17-9.10 (m, 1H), 9.04-8.99 (m, 1H), 8.92-8.74 (m, 2H), 8.64 (m, 1H), 8.46 (m, 1H), 8.41 (m, 1H), 8.24-8.17 (m, 1H), 8.04 (m, 1H), 7.73-7.64 (m, 1H), 7.10 (m, 1H), 6.91 (m, 1H), 6.70 (m, 1H), 4.70-4.60 (m, 1H), 4.23 (m, 2H), 4.13 (m, 2H), 3.81 (m, 4H), 3.46-3.40 (m, 4H), 2.46-2.40 (m, 1H), 2.38-2.29 (m, 1H), 1.96 (m, 3H), 1.47-1.35 (m, 4H).

### Example 447: (S)—N-(3-(7-(5-Acetylthiophen-2-yl)quinolin-5-yl)oxetan-3-yl)-2-methyl-5-((1-methylazetidin-2-yl)methoxy)benzamide (Compound 821)

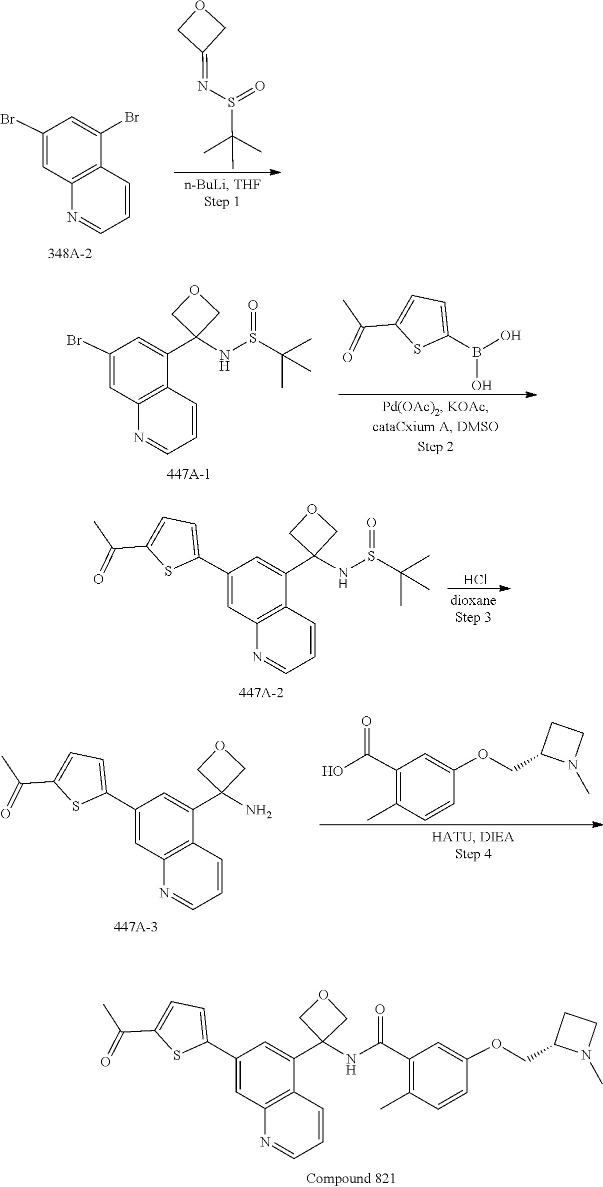

### Step 1: N-(3-(7-Bromoquinolin-5-yl)oxetan-3-yl)-2-methylpropane-2-sulfinamide (447A-1)

n-BuLi (2.5 M in Et₂O, 139 μL, 1.0 eq) was added dropwise to a solution of 5,7-dibromoquinoline (100 mg, 349 μmol, 59.5 μL, 1.0 eq) and 2-methyl-N-(oxetan-3-ylidene) propane-2-sulfinamide (61.1 mg, 349 μmol, 1.0 eq) in THF (2.0 mL) at −78° C. under a N₂ atmosphere. The resulting mixture was stirred at −78° C. for 1 h. LCMS indicated that the starting material completely consumed, and the desired mass was detected. The reaction mixture was allowed to warm to room temperature, poured into NH₄Cl aqueous (20 ml) and extracted with EtOAc (10 mL×4). The combined organic layers were dried over Na₂SO₄, filtered, and concentrated under vacuum to give a residue which was purified by flash silica gel chromatography using a gradient of EtOAc/petroleum ether from 0/1 to 1/0. N-(3-(7-Bromoquinolin-5-yl)oxetan-3-yl)-2-methylpropane-2-sulfinamide (200 mg, 522 μmol, 21% yield) was obtained as a yellow gum. ¹H NMR (400 MHZ, CDCl₃) δ 8.94 (dd, J=1.5, 4.1 Hz, 1H), 8.33 (d, J=1.1 Hz, 1H), 7.89 (d, J=8.5 Hz, 1H), 7.65 (d, J=1.8 Hz, 1H), 7.41 (dd, J=4.1, 8.6 Hz, 1H), 5.53 (d, J=7.3 Hz, 1H), 5.26-5.15 (m, 3H), 1.11 (s, 10H).

### Step 2: N-(3-(7-(5-Acetylthiophen-2-yl)quinolin-5-yl)oxetan-3-yl)-2-methylpropane-2-sulfinamide (447A-2)

To a mixture of (5-acetylthiophen-2-yl) boronic acid (63.2 mg, 372 μmol, 1.5 eq) and N-(3-(7-bromoquinolin-5-yl)oxetan-3-yl)-2-methylpropane-2-sulfinamide (95.0 mg, 248 μmol, 1.0 eq) in DMSO (4.0 mL) were added KOAc (97.3 mg, 991 μmol, 4.0 eq), cataCxium A (44.4 mg, 124 μmol, 0.5 eq) and Pd(OAc)₂ (11.1 mg, 49.6 μmol, 0.2 eq). The resulting mixture was stirred at 80° C. for 2 h under a N₂ atmosphere. LCMS indicated that the starting material completely consumed, and the desired product was detected. The reaction mixture was allowed to cool to room temperature, poured into H₂O (10 mL) and extracted with EtOAc (10 mL×2). The combined organic layers were washed with brine (10 mL×2), dried over Na₂SO₄, filtered, and concentrated under vacuum to give a residue which was purified by flash silica gel chromatography using a gradient of EtOAc/petroleum ether from 0/1 to 1/0. N-(3-(7-(5-Acetylthiophen-2-yl)quinolin-5-yl)oxetan-3-yl)-2-methylpropane-2-sulfinamide (160 mg, 373 μmol, 75% yield) was obtained as a yellow solid. ¹H NMR (400 MHZ, CDCl₃-d) δ 8.97 (dd, J=1.4, 4.1 Hz, 1H), 8.43 (s, 1H), 7.98-7.72 (m, 4H), 7.56 (d, J=4.0 Hz, 1H), 7.41 (dd, J=4.2, 8.6 Hz, 1H), 5.35-5.21 (m, 4H), 2.68-2.63 (m, 3H), 1.12 (s, 9H).

### Step 3: 1-(5-(5-(3-Aminooxetan-3-yl)quinolin-7-yl)thiophen-2-yl) ethan-1-one (447A-3)

To a solution of N-(3-(7-(5-acetylthiophen-2-yl)quinolin-5-yl)oxetan-3-yl)-2-methylpropane-2-sulfinamide (150 mg, 350 μmol, 1.0 eq) in MeOH (5.0 mL) was added HCl/dioxane (4 M, 637 μL, 7.3 eq) at 0° C. The resulting mixture was stirred at 0° C. for 30 min. LCMS indicated that the starting material completely consumed, and the desired mass was detected. The combined organic layers were dried over Na₂SO₄, filtered, and concentrated under vacuum to give the crude 1-(5-(5-(3-aminooxetan-3-yl)quinolin-7-yl)thiophen-2-yl) ethan-1-one (120 mg, 333 μmol, 95% yield, HCl salt) as a yellow solid, which was used in the next step without any further purification. ¹H NMR (400 MHZ, DMSO-d₆) δ 9.78 (br s, 3H), 9.21 (br d, J=3.6 Hz, 1H), 8.66 (br s, 1H), 8.47-8.31 (m, 1H), 8.26-8.00 (m, 3H), 7.96-7.78 (m, 1H), 5.44-5.31 (m, 4H), 2.60 (br s, 3H).

### Step 4: (S)—N-(3-(7-(5-Acetylthiophen-2-yl)quinolin-5-yl)oxetan-3-yl)-2-methyl-5-((1-methylazetidin-2-yl)methoxy)benzamide (Compound 821)

To a solution of(S)-2-methyl-5-((1-methylazetidin-2-yl)methoxy)benzoic acid (78.2 mg, 333 μmol, 1.0 eq) and 1-(5-(5-(3-aminooxetan-3-yl)quinolin-7-yl)thiophen-2-yl) ethan-1-one (120 mg, 333 μmol, 1.0 eq, HCl salt) in DMF (5.0 mL) were added HATU (316 mg, 831 μmol, 2.5 eq) and DIEA (129 mg, 998 μmol, 174 μL, 3.0 eq). The mixture was stirred at 25° C. for 5 h. LCMS indicated that the starting material was completely consumed, and the desired mass was detected. The reaction mixture was poured into H₂O (10 mL) and extracted with EtOAc (10 mL×2). The combined organic layers were dried over Na₂SO₄, filtered, and concentrated under vacuum to give a residue which was purified by preparative HPLC (Phenomenex Luna C18 column (100×30 mm, 5 μm); flow rate: 25 mL/min; gradient: 10%-40% B over 8 min; mobile phase A: 0.1% aqueous TFA, mobile phase B: acetonitrile). (S)—N-(3-(7-(5-Acetylthiophen-2-yl)quinolin-5-yl)oxetan-3-yl)-2-methyl-5-((1-methylazetidin-2-yl)methoxy)benzamide (21.6 mg, 32.9 μmol, 10% yield) was obtained as a yellow solid. M+H⁺=542.4 (LCMS); ¹H NMR (400 MHZ, CD₃OD) δ 8.96 (dd, J=1.4, 4.4 Hz, 1H), 8.36 (s, 1H), 8.31-8.24 (m, 2H), 7.95 (d, J=4.0 Hz, 1H), 7.83 (d, J=4.1 Hz, 1H), 7.63 (dd, J=4.4, 8.6 Hz, 1H), 7.15 (d, J=8.4 Hz, 1H), 6.99 (dd, J=2.8, 8.5 Hz, 1H), 6.88 (d, J=2.6 Hz, 1H), 5.47 (d, J=7.1 Hz, 2H), 5.35 (d, J=7.1 Hz, 2H), 4.74-4.63 (m, 1H), 4.35-4.16 (m, 3H), 4.02-3.92 (m, 1H), 3.00-2.92 (m, 3H), 2.62 (s, 3H), 2.58-2.48 (m, 2H), 2.04 (s, 3H).

### Example 448: (S)-2-Methyl-5-((1-methylazetidin-2-yl)methoxy)-N-(3-(7-(2-methyloxazol-5-yl)quinolin-5-yl)oxetan-3-yl)benzamide (Compound 808)

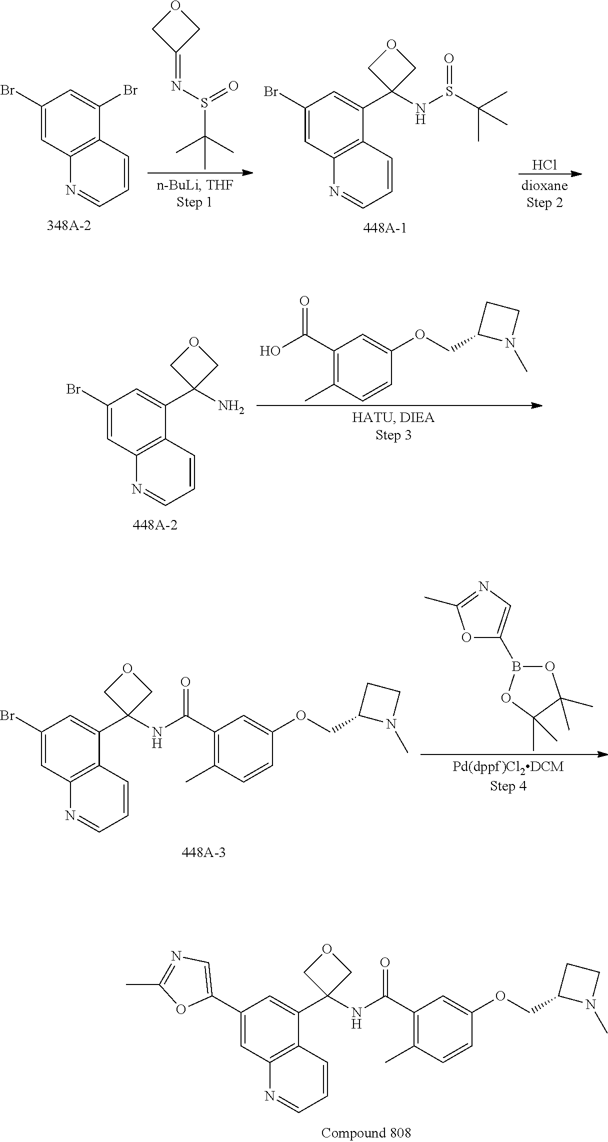

### Step 1: N-(3-(7-Bromoquinolin-5-yl)oxetan-3-yl)-2-methylpropane-2-sulfinamide (448A-1)

To a solution of 5,7-dibromoquinoline (500 mg, 1.74 mmol, 59.5 μL, 1.0 eq) in THF (10 mL) was added n-BuLi (2.5 M in hexane, 697 μL, 1.0 eq) dropwise at −78° C. under a N₂ atmosphere. The resulting mixture was stirred at −78° C. for 1 h, then a solution of 2-methyl-N-(oxetan-3-ylidene) propane-2-sulfinamide (305 mg, 1.74 mmol, 1.0 eq) in THF (1.0 mL) was added dropwise at −78° C. The resulting mixture was warmed to 25° C. for 2 h. LCMS indicated that the starting material was completely consumed, and the desired mass was detected. The mixture was poured into water (10 mL) and extracted with EtOAc (5.0 mL×2). The combined organic layers were dried over Na₂SO₄, filtered, and concentrated under vacuum to give a residue which was purified by flash silica gel chromatography using a gradient of EtOAc/petroleum ether from 0/1 to 1/0. N-(3-(7-Bromoquinolin-5-yl)oxetan-3-yl)-2-methylpropane-2-sulfinamide (150 mg, 391 μmol, 22% yield) was obtained as a yellow gum. M+H⁺=383.2 (LCMS); ¹H NMR (400 MHZ, CDCl₃) δ 8.95 (d, J=4.3 Hz, 1H), 8.48-8.36 (m, 1H), 8.06-7.89 (m, 1H), 7.69 (br s, 1H), 7.52-7.40 (m, 1H), 5.53 (d, J=7.4 Hz, 1H), 5.30-5.08 (m, 3H), 1.11 (s, 9H).

### Step 2:3-(7-Bromoquinolin-5-yl)oxetan-3-amine (448A-2)

To a solution of N-(3-(7-bromoquinolin-5-yl)oxetan-3-yl)-2-methylpropane-2-sulfinamide (150 mg, 391 μmol, 1.0 eq) in MeOH (8 mL) was added HCl/dioxane (4 M, 712 μL, 7.3 eq) at 0° C. The resulting mixture was stirred at 0° C. for 10 min. LCMS indicated that the starting material was completely consumed, and the desired mass was detected. The reaction mixture was concentrated under vacuum to give 3-(7-bromoquinolin-5-yl)oxetan-3-amine (120 mg, 380 μmol, 97% yield, HCl salt) as a yellow solid. M+H⁺=279.1 (LCMS).

### Step 3: (S)—N-(3-(7-Bromoquinolin-5-yl)oxetan-3-yl)-2-methyl-5-((1-methylazetidin-2-yl)methoxy)benzamide (448A-3)

To a solution of(S)-2-methyl-5-((1-methylazetidin-2-yl)methoxy)benzoic acid (82.0 mg, 349 μmol, 1.0 eq) and 3-(7-bromo-5-quinolyl)oxetan-3-amine (110 mg, 349 μmol, 1.0 eq, HCl salt) in DMF (5.0 mL) were added HATU (331 mg, 871 μmol, 2.5 eq) and DIEA (135 mg, 1.05 mmol, 182 μL, 3.0 eq). The mixture was stirred at 25° C. for 5 h. LCMS indicated that the starting material was completely consumed, and the desired mass was detected. The mixture was poured into water (10 mL) and extracted with EtOAc (5.0 mL×2). The combined organic layers were dried over Na₂SO₄, filtered, and concentrated under vacuum to give a residue which was purified by flash silica gel chromatography using a gradient of EtOAc/petroleum ether from 0/1 to 1/0. (S)—N-(3-(7-Bromoquinolin-5-yl)oxetan-3-yl)-2-methyl-5-((1-methylazetidin-2-yl)methoxy)benzamide (80.0 mg, 161 μmol, 46% yield) was obtained as a yellow gum. M+H⁺=496.3 (LCMS).

### Step 4: (S)-2-Methyl-5-((1-methylazetidin-2-yl)methoxy)-N-(3-(7-(2-methyloxazol-5-yl)quinolin-5-yl)oxetan-3-yl)benzamide (Compound 808)

To a solution of(S)—N-(3-(7-bromoquinolin-5-yl)oxetan-3-yl)-2-methyl-5-((1-methylazetidin-2-yl)methoxy)benzamide (60.0 mg, 121 μmol, 1.0 eq) in a mixture of dioxane (6.0 mL) and H₂O (0.6 mL) were added 2-methyl-5-(4,4,5,5-tetramethyl-1,3,2-dioxaborolan-2-yl)oxazole (37.9 mg, 181 μmol, 1.5 eq), Pd(dppf)Cl₂·CH₂Cl₂ (9.87 mg, 12.1 μmol, 0.1 eq) and Na₂CO₃ (29.5 mg, 278 μmol, 2.3 eq) under a N₂ atmosphere. The resulting mixture was stirred at 80° C. for 4 h. LCMS indicated that the starting material was completely consumed, and the desired mass was detected. The reaction mixture was allowed to cool to room temperature, poured into water (10 mL), and extracted with EtOAc (5.0 mL×2). The combined organic layers were dried over Na₂SO₄, filtered, and concentrated under vacuum to give a residue which was purified by preparative HPLC (Phenomenex luna C18 column (100× 40 mm, 5 μm); flow rate: 25 mL/min; gradient: 1%-30% B over 8 min; mobile phase A: 0.1% aqueous TFA, mobile phase B: acetonitrile). (S)-2-Methyl-5-((1-methylazetidin-2-yl)methoxy)-N-(3-(7-(2-methyloxazol-5-yl)quinolin-5-yl)oxetan-3-yl)benzamide (6.40 mg, 9.89 μmol, 8% yield, TFA salt) was obtained as a yellow solid. M+H⁺=499.4 (LCMS); ¹H NMR (400 MHZ, CD₃OD) δ 8.96 (d, J=4.1 Hz, 1H), 8.30 (s, 1H), 8.26-8.19 (m, 2H), 7.78 (s, 1H), 7.62 (dd, J=4.3, 8.6 Hz, 1H), 7.14 (d, J=8.5 Hz, 1H), 6.99 (dd, J=2.6, 8.4 Hz, 1H), 6.87 (d, J=2.5 Hz, 1H), 5.46 (br d, J=7.0 Hz, 2H), 5.34 (d, J=7.0 Hz, 2H), 4.80-4.66 (m, 2H), 4.35-4.28 (m, 1H), 4.25-4.18 (m, 1H), 3.96 (q, J=9.8 Hz, 1H), 3.05-2.91 (m, 3H), 2.62 (s, 3H), 2.59-2.51 (m, 2H), 2.01 (s, 3H).

### Example 449: 5-(Azetidin-3-ylamino)-2-methyl-N-(1-(5,6,7,8-tetrahydronaphthalen-1-yl)ethyl)benzamide (Compound 181)

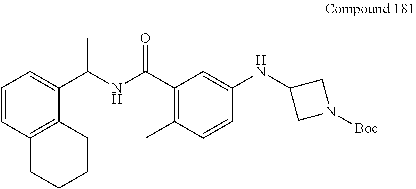 [SMILES: Cc1ccc(NC2CN(C(=O)OC(C)(C)C)C2)cc1C(=O)NC(C)c1cccc2c1CCCC2]

The synthesis of compound 181 was described in example 1 as intermediate 1A-8. M+H⁺=464.3.1 (LCMS); ¹H NMR (400 MHZ, CD₃OD) δ 7.23 (d, J=7.1 Hz, 1H), 7.12-7.07 (m, 1H), 7.03-6.95 (m, 2H), 6.56-6.49 (m, 2H), 5.43-5.36 (m, 1H), 4.61-4.58 (m, 1H), 4.29-4.17 (m, 3H), 3.74-3.69 (m, 2H), 3.10-3.02 (m, 1H), 2.83-2.77 (m, 3H), 2.21-2.18 (m, 3H), 1.95-1.87 (m, 2H), 1.83-1.77 (m, 2H), 1.48-1.46 (m, 1H), 1.46-1.45 (m, 9H), 1.31-1.30 (m, 1H).

### Example 450: (R)-2-Methyl-N-(1-(naphthalen-1-yl)ethyl)-5-nitrobenzamide (Compound 104)

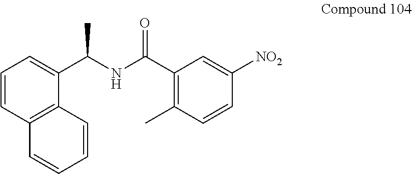 [SMILES: Cc1ccc([N+](=O)[O-])cc1C(=O)N[C@H](C)c1cccc2ccccc12]

The synthesis of compound 104 was described in example 14 as intermediate 14A-3. M+H⁺=335.2 (LCMS); ¹H NMR (400 MHZ, CDCl₃) δ 8.21 (d, J=8.5 Hz, 1H), 8.16-8.09 (m, 2H), 7.91 (d, J=7.6 Hz, 1H), 7.85 (d, J=8.1 Hz, 1H), 7.66-7.45 (m, 4H), 7.36 (d, J=8.3 Hz, 1H), 6.20-6.12 (m, 1H), 6.12-6.04 (m, 1H), 2.60-2.44 (m, 3H), 1.84 (d, J=6.5 Hz, 3H).

### Example 451: tert-Butyl 3-((3-((1-([1,1′-biphenyl]-3-yl)cyclopropyl)carbamoyl)-4-methylphenyl)amino)azetidine-1-carboxylate (Compound 188)

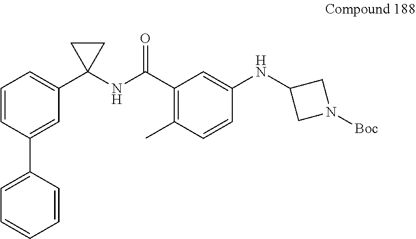 [SMILES: Cc1ccc(NC2CN(C(=O)OC(C)(C)C)C2)cc1C(=O)NC1(c2cccc(-c3ccccc3)c2)CC1]

The synthesis of compound 188 was described in example 201 as intermediate 201A-2. M+H⁺=398.1 (LCMS); ¹H NMR (400 MHZ, CDCl₃) δ 7.61-7.52 (m, 4H), 7.44 (t, J=7.7 Hz, 4H), 7.41-7.32 (m, 3H), 7.08-6.99 (m, 1H), 4.28-4.22 (m, 2H), 4.21-4.14 (m, 1H), 3.78-3.71 (m, 2H), 2.32 (s, 3H), 1.46 (br s, 2H), 1.45 (s, 9H), 1.43 (br s, 2H).

### Example 452: 2-(1-(Naphthalen-1-yl)ethyl)-7-nitro-1,2,3,4-tetrahydroisoquinoline (Compound 127)

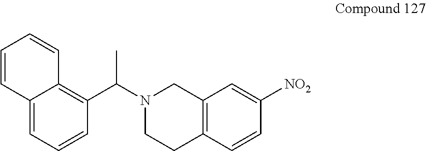 [SMILES: CC(c1cccc2ccccc12)N1CCc2ccc([N+](=O)[O-])cc2C1]

The synthesis of compound 127 was described in example 256 as intermediate 256A-1. M+H⁺=332.2 (LCMS); ¹H NMR (400 MHz, CDCl₃) δ 8.42 (br s, 1H), 8.02-7.94 (m, 1H), 7.93-7.86 (m, 2H), 7.80 (br d, J=8.1 Hz, 1H), 7.66 (br d, J=6.4 Hz, 1H), 7.53-7.42 (m, 3H), 7.23 (d, J=8.4 Hz, 1H), 4.36 (br s, 1H), 4.01 (br d, J=14.6 Hz, 1H), 3.73 (br d, J=14.9 Hz, 1H), 3.05-2.64 (m, 4H), 1.62 (br d, J=6.5 Hz, 3H).

### Example 453: (S)-5-(1-(2-Methyl-5-((1-methylazetidin-2-yl)methoxy)benzamido)cyclopropyl)quinolin-7-yl trifluoromethanesulfonate (Compound 739)

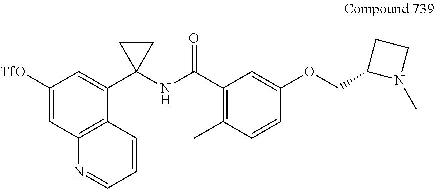 [SMILES: Cc1ccc(OC[C@@H]2CCN2C)cc1C(=O)NC1(c2cc(OS(=O)(=O)C(F)(F)F)cc3ncccc23)CC1]

The synthesis of compound 739 was described in example 352 as intermediate 352A-1. M+H⁺=550.2 (LCMS); ¹H NMR (400 MHZ, DMSO-d₆) δ 10.79-10.63 (m, 1H), 9.31 (s, 1H), 9.18 (d, J=8.6 Hz, 1H), 9.06 (dd, J=1.4, 4.1 Hz, 1H), 8.10 (d, J=2.5 Hz, 1H), 7.89 (d, J=2.5 Hz, 1H), 7.76 (dd, J=4.3, 8.6 Hz, 1H), 7.12-7.06 (m, 1H), 6.98-6.86 (m, 1H), 6.82-6.70 (m, 1H), 4.70-4.54 (m, 1H), 4.36 (dd, J=7.9, 11.2 Hz, 1H), 4.21 (dd, J=3.2, 11.3 Hz, 1H), 4.08-3.93 (m, 1H), 3.84 (br dd, J=6.6, 9.6 Hz, 1H), 2.80 (d, J=5.0 Hz, 3H), 2.41-2.24 (m, 2H), 1.93 (s, 3H), 1.47-1.37 (m, 2H), 1.33-1.25 (m, 2H).

### Example 454: 2-Methyl-N-(naphthalen-1-yl(oxetan-3-yl)methyl)-5-nitrobenzamide (Compound 110)

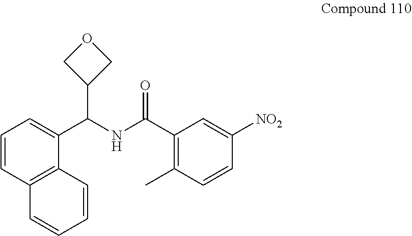

Compound 110 was synthesized according to an analogous procedure to the one described for compound 105. M+H⁺=377.1 (LCMS); ¹H NMR (400 MHZ, CDCl₃) δ 8.32 (d, J=8.5 Hz, 1H), 8.16-8.07 (m, 2H), 7.94-7.84 (m, 2H), 7.69-7.55 (m, 2H), 7.48-7.34 (m, 2H), 7.20 (d, J=7.1 Hz, 1H), 6.53 (t, J=8.8 Hz, 1H), 6.10 (br d, J=8.8 Hz, 1H), 5.13-5.00 (m, 2H), 4.89 (t, J=7.3 Hz, 1H), 4.51 (t, J=6.5 Hz, 1H), 4.00-3.79 (m, 1H), 3.50 (s, 2H), 2.54 (s, 3H).

### Example 455: 2-Methyl-N-(naphthalen-1-yl(tetrahydrofuran-3-yl)methyl)-5-nitrobenzamide (Compound 123)

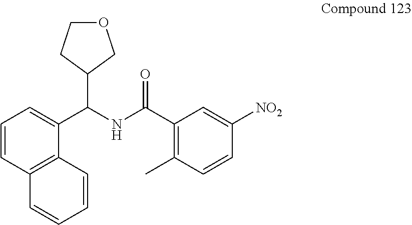

Compound 123 was synthesized according to an analogous procedure to the one described for compound 105. M+H⁺=391.0 (LCMS); ¹H NMR (400 MHZ, CDCl₃) δ 8.40-8.28 (m, 1H), 8.23-8.11 (m, 2H), 7.94 (dd, J=4.6, 7.3 Hz, 1H), 7.91-7.84 (m, 1H), 7.68-7.48 (m, 4H), 7.44-7.35 (m, 1H), 6.22-6.03 (m, 1H), 4.17-4.01 (m, 1H), 3.99-3.76 (m, 2H), 3.56-3.43 (m, 1H), 3.29-3.03 (m, 1H), 2.51 (d, J=9.8 Hz, 3H), 2.29 (q, J=7.3 Hz, 1H), 2.25-1.76 (m, 1H).

### Example 456: N-(3-Methoxy-1-(naphthalen-1-yl) propyl)-2-methyl-5-nitrobenzamide (Compound 113)

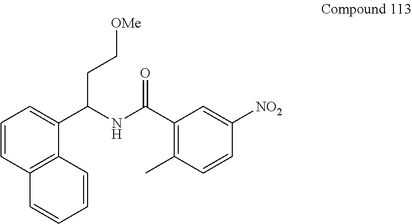

Compound 113 was synthesized according to an analogous procedure to the one described for compound 108. M+H⁺=379.2 (LCMS); ¹H NMR (400 MHZ, CDCl₃) δ 8.35 (d, J=2.4 Hz, 1H), 8.22-8.15 (m, 2H), 7.91 (d, J=7.6 Hz, 1H), 7.82 (d, J=7.7 Hz, 1H), 7.62-7.46 (m, 4H), 7.45-7.37 (m, 2H), 6.21 (dt, J=4.6, 7.1 Hz, 1H), 3.58-3.42 (m, 2H), 3.40 (s, 3H), 2.55 (s, 3H), 2.51-2.20 (m, 2H).

### Example 457: 5-Amino-2-methyl-N-(naphthalen-1-yl(oxetan-3-yl)methyl)benzamide (Compound 114)

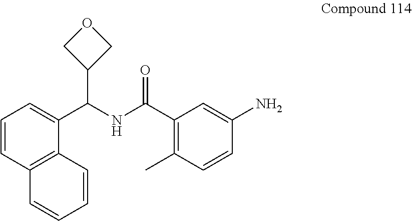

Compound 114 was synthesized according to an analogous procedure to the one described for compound 109. M+H⁺=347.0 (LCMS); ¹H NMR (400 MHZ, CDCl₃) δ 8.33 (d, J=8.4 Hz, 1H), 7.94-7.80 (m, 2H), 7.64-7.53 (m, 2H), 7.44-7.35 (m, 1H), 7.13 (d, J=7.1 Hz, 1H), 6.94 (d, J=8.0 Hz, 1H), 6.66-6.53 (m, 2H), 6.45 (t, J=8.7 Hz, 1H), 6.10 (br d, J=9.3 Hz, 1H), 5.11-4.91 (m, 2H), 4.83 (dd, J=6.6, 8.0 Hz, 1H), 4.52 (t, J=6.6 Hz, 1H), 3.94-3.71 (m, 1H), 2.34-2.24 (m, 3H).

### Example 458: 5-Amino-N-(3-methoxy-1-(naphthalen-1-yl) propyl)-2-methylbenzamide (Compound 117)

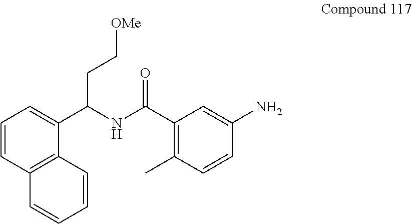

Compound 117 was synthesized according to an analogous procedure to the one described for compound 118. M+H⁺=349.2 (LCMS); ¹H NMR (400 MHZ, CDCl₃) δ 8.20 (d, J=8.3 Hz, 1H), 7.87 (d, J=8.0 Hz, 1H), 7.77 (br d, J=8.0 Hz, 1H), 7.59-7.37 (m, 4H), 7.18-7.04 (m, 1H), 6.92 (br d, J=7.6 Hz, 1H), 6.89-6.68 (m, 2H), 6.14 (br d, J=4.6 Hz, 1H), 3.49-3.37 (m, 2H), 3.33-3.28 (m, 3H), 2.33 (br d, J=6.6 Hz, 1H), 2.27 (s, 3H), 2.21 (br d, J=10.4 Hz, 1H).

### Example 459: 5-(Azetidin-3-ylamino)-2-methyl-N-(1-(1,2,3,4-tetrahydro naphthalen-1-yl)ethyl)benzamide (Compound 182)

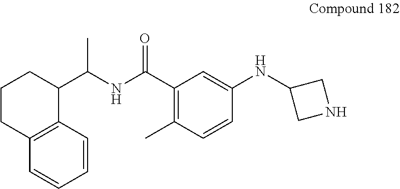

Compound 182 was synthesized according to an analogous procedure to the one described for compound 172. M+H⁺=364.1 (LCMS); ¹H NMR (400 MHZ, CDCl₃) δ 9.99-9.63 (m, 2H), 7.35-7.28 (m, 1H), 7.18-7.06 (m, 3H), 7.03-6.89 (m, 1H), 6.50-6.24 (m, 2H), 6.05-5.68 (m, 1H), 4.83-4.63 (m, 1H), 4.40-4.04 (m, 3H), 3.85-3.59 (m, 2H), 3.28-2.99 (m, 1H), 2.84 (br d, J=2.1 Hz, 1H), 2.26-1.87 (m, 5H), 1.83-1.47 (m, 2H), 1.36-1.10 (m, 3H).

### Example 460: 2-(Aminomethyl)-N-(1-(7-methoxyquinolin-5-yl)cyclopropyl)-5-methylbenzofuran-6-carboxamide (Compound 607)

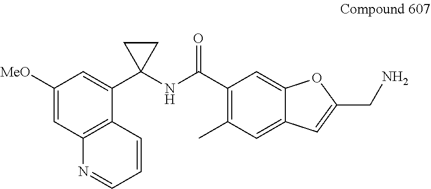

Compound 607 was synthesized according to an analogous procedure to the one described for compound 197. M+H⁺=402.1 (LCMS); ¹H NMR (400 MHZ, DMSO-d₆) δ 9.12 (s, 1H), 8.96 (d, J=8.4 Hz, 1H), 8.83 (dd, J=1.4, 4.1 Hz, 1H), 7.48 (d, J=2.6 Hz, 1H), 7.44 (dd, J=4.2, 8.4 Hz, 1H), 7.34-7.28 (m, 2H), 7.20 (s, 1H), 6.60 (s, 1H), 3.93 (s, 3H), 3.79 (s, 2H), 2.11 (s, 3H), 1.40-1.32 (m, 2H), 1.23-1.18 (m, 2H).

### Example 461: 2-((Dimethylamino)methyl)-N-(1-(7-methoxyquinolin-5-yl)cyclopropyl)-5-methylbenzofuran-6-carboxamide (Compound 606)

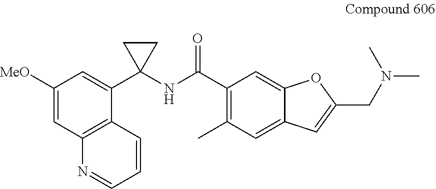

Compound 606 was synthesized according to an analogous procedure to the one described for compound 198. M+H⁺=364.1 (LCMS); ¹H NMR (400 MHZ, DMSO-d₆) δ=11.26 (br d, J=2.5 Hz, 1H), 9.62 (br d, J=8.6 Hz, 1H), 9.42 (s, 1H), 9.16 (d, J=5.0 Hz, 1H), 7.93 (dd, J=5.3, 8.4 Hz, 1H), 7.72 (d, J=2.4 Hz, 1H), 7.66 (d, J=1.9 Hz, 1H), 7.48 (s, 1H), 7.38 (s, 1H), 7.14 (s, 1H), 4.51 (s, 2H), 4.02 (s, 3H), 2.73 (s, 6H), 2.13 (s, 3H), 1.44 (br s, 2H), 1.32 (br s, 2H).

### Example 462: 5-(2-Aminoethoxy)-N-(1-(7-methoxyquinolin-5-yl)cyclopropyl)-2-methylbenzamide (Compound 513)

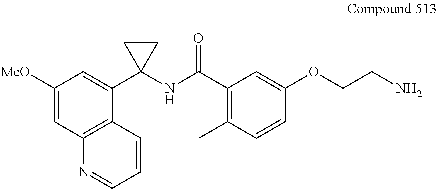

Compound 513 was synthesized according to an analogous procedure to the one described for compound 215. M+H⁺=392.1 (LCMS); ¹H NMR (400 MHZ, DMSO-d₆) δ 9.12-9.07 (m, 1H), 8.94 (dd, J=1.0, 8.4 Hz, 1H), 8.82 (dd, J=1.6, 4.2 Hz, 1H), 7.47 (d, J=2.6 Hz, 1H), 7.42 (dd, J=4.3, 8.5 Hz, 1H), 7.31 (d, J=2.4 Hz, 1H), 7.03 (d, J=8.5 Hz, 1H), 6.83 (dd, J=2.7, 8.3 Hz, 1H), 6.61 (d, J=2.8 Hz, 1H), 3.92 (s, 3H), 3.82 (t, J=5.8 Hz, 2H), 2.80 (t, J=5.8 Hz, 2H), 1.94 (s, 3H), 1.38-1.29 (m, 2H), 1.23-1.14 (m, 2H).

### Example 463: 5-(Azetidin-3-ylmethoxy)-2-methyl-N-(1-(naphthalen-1-yl)cyclopropyl)benzamide (Compound 430)

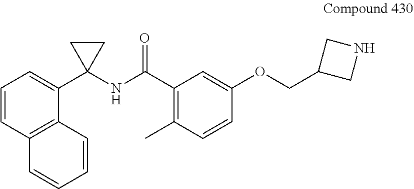

Compound 430 was synthesized according to an analogous procedure to the one described for compound 263. M+H⁺=387.1 (LCMS); ¹H NMR (400 MHz, DMSO-d₆) δ 9.12-9.07 (m, 1H), 8.79-8.62 (m, 3H), 7.98-7.91 (m, 1H), 7.86-7.79 (m, 2H), 7.61-7.42 (m, 3H), 7.11-7.03 (m, 1H), 6.91-6.83 (m, 1H), 6.71-6.65 (m, 1H), 4.07-3.99 (m, 4H), 3.82-3.71 (m, 2H), 3.18-3.05 (m, 1H), 2.03-1.90 (m, 3H), 1.40-1.31 (m, 2H), 1.23-1.13 (m, 2H).

### Example 464: 5-(((1R,2S)-2-Aminocyclohexyl)oxy)-2-methyl-N-(1-(naphthalen-1-yl)cyclopropyl)benzamide (Compound 428)

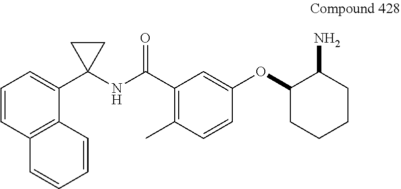

Compound 428 was synthesized according to an analogous procedure to the one described for compound 263. M+H⁺=415.1 (LCMS); ¹H NMR (400 MHZ, DMSO-d₆) δ 9.14-9.08 (m, 1H), 8.70-8.62 (m, 1H), 8.02-7.88 (m, 4H), 7.87-7.78 (m, 2H), 7.61-7.41 (m, 3H), 7.11-7.03 (m, 1H), 6.96-6.89 (m, 1H), 6.76-6.70 (m, 1H), 4.53-4.44 (m, 1H), 3.36 (br s, 1H), 2.01-1.93 (m, 3H), 1.92-1.84 (m, 1H), 1.77-1.65 (m, 3H), 1.50-1.32 (m, 6H), 1.22-1.12 (m, 2H).

### Example 465: 5-(((1R,2R)-2-Aminocyclohexyl)oxy)-2-methyl-N-(1-(naphthalen-1-yl)cyclopropyl)benzamide (Compound 429)

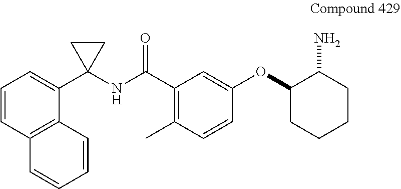

Compound 429 was synthesized according to an analogous procedure to the one described for compound 263. M+H⁺=415.1 (LCMS); ¹H NMR (400 MHZ, DMSO-d₆) δ 9.15-9.09 (m, 1H), 8.69-8.62 (m, 1H), 8.02-7.78 (m, 6H), 7.63-7.41 (m, 3H), 7.11-7.03 (m, 1H), 6.95-6.88 (m, 1H), 6.71-6.64 (m, 1H), 4.14-3.97 (m, 1H), 3.20-3.07 (m, 1H), 2.12-2.03 (m, 1H), 1.98 (s, 4H), 1.74-1.61 (m, 2H), 1.38-1.15 (m, 8H).

### Example 466: 5-(2-(Dimethylamino)ethoxy)-N-(1-(2-methoxyphenyl)cyclopropyl)-2-methylbenzamide (Compound 277)

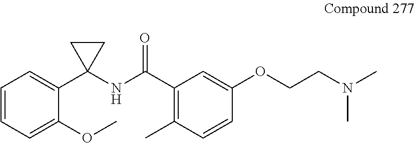

Compound 277 was synthesized according to an analogous procedure to the one described for compound 271. M+H⁺=369.1 (LCMS); ¹H NMR (400 MHZ, DMSO-d₆) δ 9.04-8.75 (m, 1H), 7.26-7.14 (m, 3H), 7.00-6.91 (m, 2H), 6.86 (d, J=8.9 Hz, 2H), 4.40-4.25 (m, 2H), 3.73 (s, 3H), 3.55-3.44 (m, 2H), 2.84 (s, 6H), 2.22 (s, 3H), 1.17 (br d, J=6.5 Hz, 4H).

### Example 467: 5-(2-(Dimethylamino)ethoxy)-N-(1-(3-methoxyphenyl)cyclopropyl)-2-methylbenzamide (Compound 278)

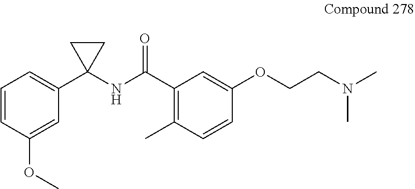

Compound 278 was synthesized according to an analogous procedure to the one described for compound 271. M+H⁺=369.1 (LCMS); ¹H NMR (400 MHZ, DMSO-d₆) δ 7.33 (d, J=8.8 Hz, 2H), 7.23-7.14 (m, 1H), 7.04-6.93 (m, 2H), 6.87 (d, J=8.8 Hz, 2H), 4.43-4.26 (m, 2H), 3.78 (s, 3H), 3.66-3.51 (m, 2H), 2.98 (s, 6H), 2.27 (s, 3H), 1.17-1.34 (m, 4H).

### Example 468: (S)-2-Methyl-5-((1-methylazetidin-2-yl)methoxy)-N-(1-(phenanthren-9-yl)cyclopropyl)benzamide (Compound 569)

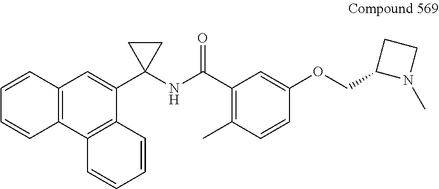

Compound 569 was synthesized according to an analogous procedure to the one described for compound 272. M+H⁺=451.1 (LCMS); ¹H NMR (400 MHZ, DMSO-d₆) δ 9.10 (s, 1H), 8.80 (br d, J=7.6 Hz, 3H), 8.12 (s, 1H), 8.03-7.96 (m, 1H), 7.72-7.62 (m, 4H), 7.04 (d, J=8.5 Hz, 1H), 6.85 (br d, J=2.5 Hz, 1H), 6.63 (d, J=2.5 Hz, 1H), 3.96 (br d, J=3.4 Hz, 2H), 2.89 (s, 2H), 2.73 (s, 2H), 2.40 (br s, 3H), 2.02-1.90 (m, 4H), 1.46-1.37 (m, 2H), 1.28 (br s, 2H).

### Example 469: 2-Methyl-5-((1-methylazetidin-3-yl)methoxy)-N-(1-(naphthalen-1-yl)cyclopropyl)benzamide (Compound 427)

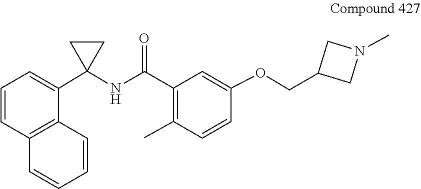 [SMILES: Cc1ccc(OCC2CN(C)C2)cc1C(=O)NC1(c2cccc3ccccc23)CC1]

Compound 427 was synthesized according to an analogous procedure to the one described for compound 283. M+H⁺=401.1 (LCMS); ¹H NMR (400 MHZ, DMSO-d₆) δ 10.39-9.96 (m, 1H), 9.14-9.06 (m, 1H), 8.70-8.61 (m, 1H), 7.99-7.90 (m, 1H), 7.89-7.73 (m, 2H), 7.64-7.40 (m, 3H), 7.15-7.01 (m, 1H), 6.93-6.82 (m, 1H), 6.74-6.61 (m, 1H), 4.28-4.17 (m, 1H), 4.11-4.06 (m, 1H), 4.05-3.95 (m, 3H), 3.81-3.75 (m, 1H), 3.13-3.04 (m, 1H), 2.81-2.76 (m, 3H), 2.02-1.88 (m, 3H), 1.41-1.29 (m, 2H), 1.23 (br s, 1H).

### Example 470: 5-(2-(Dimethylamino)ethoxy)-N-(1-(3-hydroxyphenyl)cyclopropyl)-2-methylbenzamide (Compound 306)

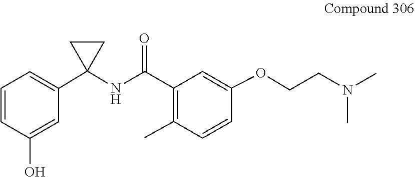 [SMILES: Cc1ccc(OCCN(C)C)cc1C(=O)NC1(c2cccc(O)c2)CC1]

Compound 306 was synthesized according to an analogous procedure to the one described for compound 294. M+H⁺=355.1 (LCMS); ¹H NMR (400 MHZ, DMSO-d₆) δ 10.38-10.09 (m, 1H), 9.30 (br s, 1H), 8.91 (s, 1H), 7.18 (br d, J=8.0 Hz, 1H), 7.07 (br t, J=7.9 Hz, 1H), 7.07 (br t, J=7.9 Hz, 1H), 7.03-6.93 (m, 2H), 6.69 (s, 1H), 6.59 (br dd, J=7.9, 13.5 Hz, 2H), 4.35 (br t, J=4.6 Hz, 2H), 3.50 (br d, J=4.5 Hz, 2H), 2.84 (br d, J=4.4 Hz, 6H), 2.17-2.31 (m, 3H), 1.21 (br s, 4H).

### Example 471: 5-(2-Amino-3-methylbutoxy)-N-(1-(7-methoxyquinolin-5-yl)cyclopropyl)-2-methylbenzamide (Compound 572)

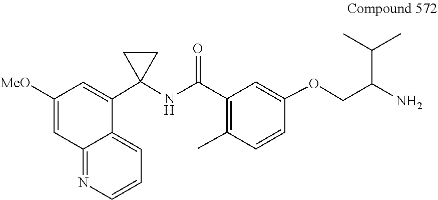 [SMILES: COc1cc(C2(NC(=O)c3cc(OCC(N)C(C)C)ccc3C)CC2)c2cccnc2c1]

Compound 572 was synthesized according to an analogous procedure to the one described for compound 324. M+H⁺=434.1 (LCMS); ¹H NMR (400 MHZ, DMSO-d₆) δ 9.48 (br d, J=4.0 Hz, 1H), 9.28 (s, 1H), 9.10 (br d, J=5.0 Hz, 1H), 8.12 (br d, J=2.1 Hz, 3H), 7.88-7.80 (m, 1H), 7.67 (d, J=2.1 Hz, 1H), 7.54 (d, J=1.6 Hz, 1H), 7.09 (d, J=8.5 Hz, 1H), 6.93 (dd, J=2.8, 8.4 Hz, 1H), 6.75 (d, J=2.8 Hz, 1H), 4.15-4.10 (m, 1H), 4.00 (s, 3H), 3.98 (br s, 1H), 3.23 (br d, J=4.1 Hz, 1H), 2.05-1.98 (m, 1H), 1.95 (s, 3H), 1.39 (br s, 2H), 1.29 (br s, 2H), 0.99 (d, J=6.9 Hz, 3H), 0.95 (d, J=6.8 Hz, 3H).

### Example 472: (S)-5-((1-(2-Fluoroethyl)azetidin-2-yl)methoxy)-N-(1-(7-methoxyquinolin-5-yl)cyclopropyl)-2-methylbenzamide (Compound 567)

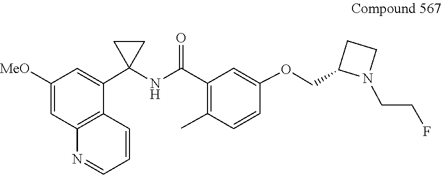 [SMILES: COc1cc(C2(NC(=O)c3cc(OC[C@@H]4CCN4CCF)ccc3C)CC2)c2cccnc2c1]

Compound 567 was synthesized according to an analogous procedure to the one described for compound 348. M+H⁺=464.2. 1H NMR (400 MHZ, DMSO-d₆) δ 9.09 (s, 1H), 8.94 (br d, J=8.3 Hz, 1H), 8.81 (br d, J=3.4 Hz, 1H), 8.40-8.11 (m, 1H), 7.50-7.38 (m, 2H), 7.31 (d, J=1.8 Hz, 1H), 7.03 (br d, J=8.6 Hz, 1H), 6.93-6.78 (m, 1H), 6.60 (d, J=2.1 Hz, 1H), 4.42 (br t, J=4.6 Hz, 1H), 4.30 (br t, J=4.8 Hz, 1H), 4.02-3.76 (m, 6H), 2.97-2.73 (m, 3H), 2.59 (br d, J=3.0 Hz, 1H), 2.10-1.81 (m, 5H), 1.44-1.28 (m, 2H), 1.23-1.09 (m, 2H).

### Example 473: 5-(2-((2-Fluoroethyl)(methyl)amino)ethoxy)-N-(1-(7-methoxyquinolin-5-yl)cyclopropyl)-2-methylbenzamide (Compound 571)

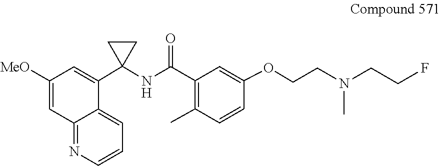 [SMILES: COc1cc(C2(NC(=O)c3cc(OCCN(C)CCF)ccc3C)CC2)c2cccnc2c1]

Compound 571 was synthesized according to an analogous procedure to the one described for compound 348. M+H⁺=452.1 (LCMS); ¹H NMR (400 MHZ, DMSO-d₆) δ 10.82-10.62 (m, 1H), 9.63-9.49 (m, 1H), 9.31 (s, 1H), 9.13 (br d, J=4.5 Hz, 1H), 7.97-7.82 (m, 1H), 7.69 (d, J=2.4 Hz, 1H), 7.57 (d, J=1.5 Hz, 1H), 7.10 (d, J=8.6 Hz, 1H), 6.92 (dd, J=2.6, 8.4 Hz, 1H), 6.75 (d, J=2.6 Hz, 1H), 4.97-4.79 (m, 2H), 4.31 (s, 2H), 4.01 (s, 3H), 3.72-3.60 (m, 4H), 2.86 (br d, J=2.3 Hz, 3H), 1.96 (s, 3H), 1.44-1.36 (m, 2H), 1.30 (br s, 2H).

### Example 474: N-(1-(7-Fluoronaphthalen-1-yl)cyclopropyl)-2-methyl-5-((1-methylazetidin-2-yl)methoxy)benzamide (Compound 444)

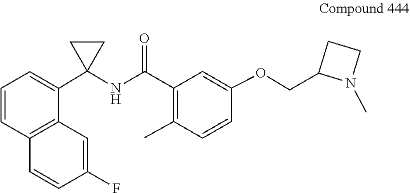 [SMILES: Cc1ccc(OCC2CCN2C)cc1C(=O)NC1(c2cccc3ccc(F)cc23)CC1]

Compound 444 was synthesized according to an analogous procedure to the one described for compound 365. M+H⁺=419.1 (LCMS); ¹H NMR (400 MHZ, CD₃OD) δ 8.57-8.46 (m, 1H), 8.34-8.23 (m, 1H), 8.01-7.90 (m, 2H), 7.87-7.80 (m, 1H), 7.47-7.39 (m, 1H), 7.36-7.27 (m, 1H), 7.12-7.04 (m, 1H), 6.93-6.87 (m, 1H), 6.73-6.67 (m, 1H), 4.30-4.03 (m, 3H), 3.92-3.79 (m, 1H), 3.66-3.51 (m, 1H), 2.76-2.67 (m, 3H), 2.43-2.28 (m, 2H), 2.05-1.95 (m, 3H), 1.48-1.42 (m, 2H), 1.34-1.26 (m, 2H).

### Example 475: 5-((Hexahydro-1H-pyrrolizin-7a-yl)methoxy)-N-(1-(7-methoxyquinolin-5-yl)cyclopropyl)-2-methylbenzamide (Compound 559)

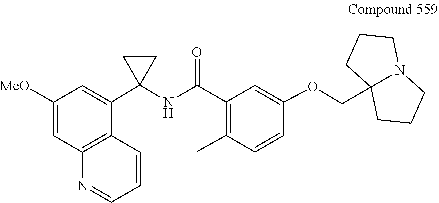 [SMILES: COc1cc(C2(NC(=O)c3cc(OCC45CCCN4CCC5)ccc3C)CC2)c2cccnc2c1]

Compound 559 was synthesized according to an analogous procedure to the one described for compound 376. M+H⁺=472.3 (LCMS); ¹H NMR (400 MHZ, DMSO-d₆) δ 9.08 (s, 1H), 8.99-8.94 (m, 1H), 8.81 (dd, J=1.6, 4.2 Hz, 1H), 7.47 (d, J=2.6 Hz, 1H), 7.41 (dd, J=4.3, 8.5 Hz, 1H), 7.31 (d, J=2.5 Hz, 1H), 7.01 (d, J=8.5 Hz, 1H), 6.82 (dd, J=2.8, 8.4 Hz, 1H), 6.61 (d, J=2.8 Hz, 1H), 3.92 (s, 3H), 3.52 (s, 2H), 2.93-2.82 (m, 2H), 2.58-2.51 (m, 2H), 1.93 (s, 3H), 1.85-1.63 (m, 6H), 1.56-1.47 (m, 2H), 1.38-1.30 (m, 2H), 1.21-1.16 (m, 2H).

### Example 476: 5-(((2R,7aS)-2-Fluorohexahydro-1H-pyrrolizin-7a-yl)methoxy)-N-(1-(7-methoxyquinolin-5-yl)cyclopropyl)-2-methylbenzamide (Compound 596)

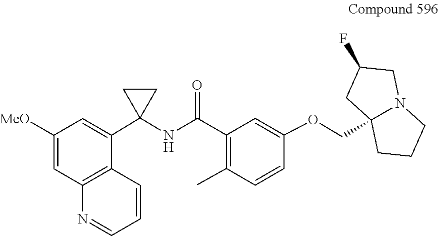 [SMILES: COc1cc(C2(NC(=O)c3cc(OC[C@@]45CCCN4C[C@H](F)C5)ccc3C)CC2)c2cccnc2c1]

Compound 596 was synthesized according to an analogous procedure to the one described for compound 376. M+H⁺=490.1 (LCMS); ¹H NMR (400 MHZ, CD₃OD) δ 9.83 (br d, J=8.1 Hz, 1H), 9.08 (d, J=5.5 Hz, 1H), 8.03-7.88 (m, 2H), 7.46 (br s, 1H), 7.12 (d, J=8.3 Hz, 1H), 6.97 (br d, J=8.7 Hz, 1H), 6.86 (d, J=2.4 Hz, 1H), 5.67-5.46 (m, 1H), 4.27-3.99 (m, 5H), 3.95-3.70 (m, 3H), 3.52-3.41 (m, 1H), 2.76-2.44 (m, 2H), 2.41-2.16 (m, 3H), 2.01 (d, J=2.1 Hz, 3H), 1.63-1.53 (m, 2H), 1.48-1.36 (m, 3H).

### Example 477: N-(1-(Isoquinolin-1-yl)cyclopropyl)-2-methyl-5-((1-methylazetidin-2-yl)methoxy)benzamide (Compound 454)

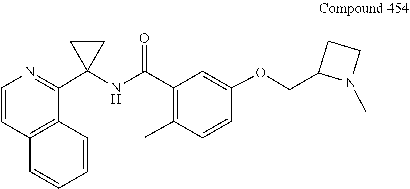 [SMILES: Cc1ccc(OCC2CCN2C)cc1C(=O)NC1(c2nccc3ccccc23)CC1]

Compound 454 was synthesized according to an analogous procedure to the one described for compound 384. M+H⁺=402.1 (LCMS); ¹H NMR (400 MHZ, DMSO-d₆) δ 9.25 (s, 1H), 8.82 (d, J=8.3 Hz, 1H), 8.38 (d, J=5.6 Hz, 1H), 7.97 (d, J=8.1 Hz, 1H), 7.83-7.54 (m, 3H), 7.10-6.96 (m, 1H), 6.90-6.81 (m, 1H), 6.68 (d, J=2.7 Hz, 1H), 3.89 (d, J=5.4 Hz, 2H), 3.31-3.24 (m, 2H), 2.78-2.70 (m, 1H), 2.24 (s, 3H), 2.02-1.82 (m, 5H), 1.62-1.46 (m, 2H), 1.43-1.27 (m, 2H).

### Example 478: 2-Methyl-5-((1-methylazetidin-2-yl)methoxy)-N-(1-(quinolin-4-yl)cyclopropyl)benzamide (Compound 473)

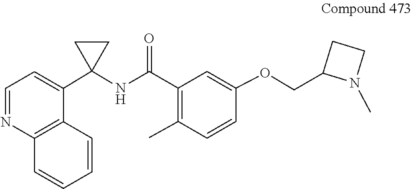 [SMILES: Cc1ccc(OCC2CCN2C)cc1C(=O)NC1(c2ccnc3ccccc23)CC1]

Compound 473 was synthesized according to an analogous procedure to the one described for compound 384. M+H⁺=402.1 (LCMS); ¹H NMR (400 MHZ, DMSO-d₆) δ 9.20 (s, 1H), 8.87 (d, J=4.4 Hz, 1H), 8.66 (d, J=7.6 Hz, 1H), 8.05 (d, J=7.8 Hz, 1H), 7.76 (s, 1H), 7.70 (d, J=4.4 Hz, 1H), 7.68-7.60 (m, 1H), 7.03 (d, J=8.4 Hz, 1H), 6.84 (dd, J=2.8, 8.4 Hz, 1H), 6.63 (d, J=2.8 Hz, 1H), 3.86 (d, J=5.4 Hz, 2H), 3.28-3.11 (m, 2H), 2.71 (br dd, J=1.3, 7.9 Hz, 1H), 2.21 (s, 3H), 2.03-1.94 (m, 1H), 1.93 (s, 3H), 1.91-1.78 (m, 1H), 1.38 (d, J=1.9 Hz, 2H), 1.25 (d, J=1.9 Hz, 2H).

### Example 479: N-(1-(3-Methoxyisoquinolin-5-yl)cyclopropyl)-2-methyl-5-((1-methylazetidin-2-yl)methoxy)benzamide (Compound 456)

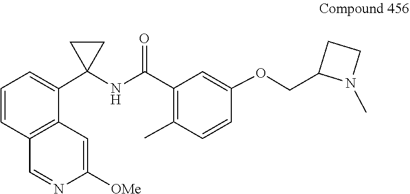 [SMILES: COc1cc2c(C3(NC(=O)c4cc(OCC5CCN5C)ccc4C)CC3)cccc2cn1]

Compound 456 was synthesized according to an analogous procedure to the one described for compound 384. M+H⁺=432.1 (LCMS); ¹H NMR (400 MHZ, CD₃OD) δ 8.97 (s, 1H), 8.07-7.97 (m, 1H), 7.92 (d, J=8.25 Hz, 1H), 7.80 (s, 1H), 7.43-7.37 (m, 1H), 7.04 (d, J=8.38 Hz, 1H), 6.84 (dd, J=8.38, 2.75 Hz, 1H), 6.66 (d, J=2.63 Hz, 1H), 4.05 (s, 3H), 3.97-3.91 (m, 2H), 3.64-3.55 (m, 1H), 3.46 (td, J=7.66, 3.19 Hz, 1H), 3.04 (q, J=8.34 Hz, 1H), 2.49-2.40 (m, 3H), 2.14-2.06 (m, 2H), 2.01 (s, 3H), 1.49-1.43 (m, 2H), 1.30-1.26 (m, 2H).

### Example 480: 2-Methyl-5-((1-methylazetidin-2-yl)methoxy)-N-(1-(2-methylnaphthalen-1-yl)cyclopropyl)benzamide (Compound 458)

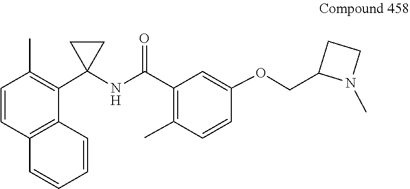 [SMILES: Cc1ccc(OCC2CCN2C)cc1C(=O)NC1(c2c(C)ccc3ccccc23)CC1]

Compound 458 was synthesized according to an analogous procedure to the one described for compound 384. M+H⁺=415.1 (LCMS); ¹H NMR (400 MHZ, CD₃OD) δ 8.99-8.82 (m, 2H), 7.80 (s, 1H), 7.73 (d, J=8.4 Hz, 1H), 7.59-7.49 (m, 1H), 7.47-7.39 (m, 1H), 7.32 (d, J=8.4 Hz, 1H), 7.13 (d, J=8.4 Hz, 1H), 6.96 (dd, J=2.8, 8.4 Hz, 1H), 6.79 (d, J=2.8 Hz, 1H), 4.74-4.59 (m, 1H), 4.33-4.25 (m, 1H), 4.24-4.12 (m, 2H), 3.96 (q, J=9.6 Hz, 1H), 2.93 (d, J=13.6 Hz, 6H), 2.62-2.50 (m, 2H), 2.10 (s, 3H), 1.82-1.72 (m, 1H), 1.66-1.56 (m, 1H), 1.44-1.34 (m, 1H), 1.24-1.14 (m, 1H).

### Example 481: N-(1-(Anthracen-9-yl)cyclopropyl)-2-methyl-5-((1-methylazetidin-2-yl)methoxy)benzamide (Compound 507)

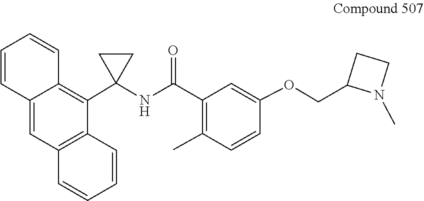 [SMILES: Cc1ccc(OCC2CCN2C)cc1C(=O)NC1(c2c3ccccc3cc3ccccc23)CC1]

Compound 507 was synthesized according to an analogous procedure to the one described for compound 384. M+H⁺=451.1 (LCMS); ¹H NMR (400 MHZ, DMSO-d₆) δ 9.74 (s, 1H), 8.62 (s, 1H), 8.18-8.09 (m, 4H), 7.57-7.50 (m, 4H), 7.09-7.01 (m, 1H), 6.92-6.74 (m, 3H), 3.93-3.83 (m, 2H), 3.26 (br d, J=4.6 Hz, 1H), 2.30-2.15 (m, 7H), 2.05-1.79 (m, 3H), 1.25 (d, J=7.0 Hz, 4H).

### Example 482: N-(1-(7-Hydroxyquinolin-5-yl)cyclopropyl)-2-methyl-5-((1-methylazetidin-2-yl)methoxy)benzamide (Compound 488)

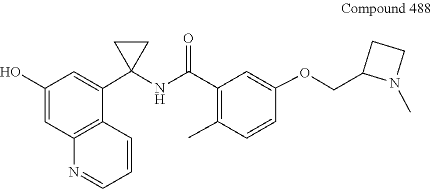 [SMILES: Cc1ccc(OCC2CCN2C)cc1C(=O)NC1(c2cc(O)cc3ncccc23)CC1]

Compound 488 was synthesized according to an analogous procedure to the one described for compound 433. M+H⁺=418.1 (LCMS); ¹H NMR (400 MHZ, DMSO-d₆) δ 10.12 (s, 1H), 9.08 (s, 1H), 8.89 (d, J=7.9 Hz, 1H), 8.73 (dd, J=1.5, 4.3 Hz, 1H), 7.45 (d, J=2.4 Hz, 1H), 7.32 (dd, J=4.2, 8.4 Hz, 1H), 7.15 (d, J=2.4 Hz, 1H), 7.03 (d, J=8.4 Hz, 1H), 6.84 (dd, J=2.8, 8.4 Hz, 1H), 6.61 (d, J=2.8 Hz, 1H), 3.88 (d, J=5.4 Hz, 2H), 3.27 (br t, J=6.6 Hz, 2H), 2.84-2.72 (m, 1H), 2.23 (s, 3H), 2.01-1.78 (m, 5H), 1.37-1.30 (m, 2H), 1.20-1.11 (m, 2H).

### Example 483: (S)—N-(1-(2-Chloro-7-methoxyquinolin-5-yl)cyclopropyl)-2-methyl-5-((1-methylazetidin-2-yl)methoxy)benzamide (Compound 617)

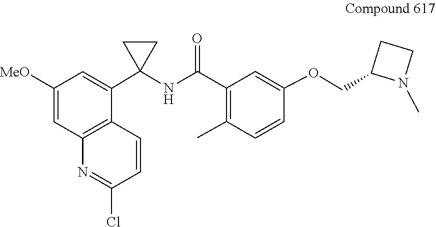 [SMILES: COc1cc(C2(NC(=O)c3cc(OC[C@@H]4CCN4C)ccc3C)CC2)c2ccc(Cl)nc2c1]

Compound 617 was synthesized according to an analogous procedure to the one described for compound 436. M+H⁺=466.2 (LCMS); ¹H NMR (400 MHZ, DMSO-d₆) δ 9.11 (s, 1H), 8.99 (d, J=8.8 Hz, 1H), 7.49 (dd, J=3.1, 5.6 Hz, 2H), 7.29 (d, J=2.5 Hz, 1H), 7.03 (d, J=8.5 Hz, 1H), 6.84 (dd, J=2.8, 8.4 Hz, 1H), 6.61 (d, J=2.8 Hz, 1H), 3.92 (s, 3H), 3.87 (d, J=5.4 Hz, 2H), 3.28-3.19 (m, 2H), 2.78-2.68 (m, 1H), 2.21 (s, 3H), 1.99-1.80 (m, 5H), 1.33 (s, 2H), 1.25-1.17 (m, 2H).

### Example 484: 5-(2-Aminopropoxy)-2-methyl-N-(1-(quinolin-5-yl)cyclopropyl)benzamide (Compound 498)

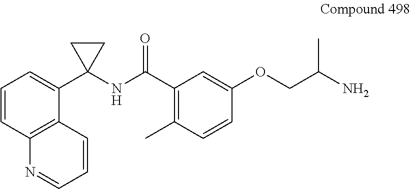 [SMILES: Cc1ccc(OCC(C)N)cc1C(=O)NC1(c2cccc3ncccc23)CC1]

Compound 498 was synthesized according to an analogous procedure to the one described for compound 445. M+H⁺=376.1 (LCMS); ¹H NMR (400 MHZ, DMSO-d₆) δ 9.22-9.13 (m, 2H), 8.98 (dd, J=4.25, 1.38 Hz, 1H), 8.01-7.86 (m, 5H), 7.78 (dd, J=8.38, 7.25 Hz, 1H), 7.68 (dd, J=8.63, 4.25 Hz, 1H), 7.08 (d, J=8.50 Hz, 1H), 6.89 (dd, J=8.38, 2.75 Hz, 1H), 6.67 (d, J=2.75 Hz, 1H), 4.02 (dd, J=10.26, 3.88 Hz, 1H), 3.85 (dd, J=10.38, 7.13 Hz, 1H), 3.60-3.48 (m, 1H), 1.95 (s, 3H), 1.41-1.34 (m, 2H), 1.26-1.18 (m, 5H).

### Example 485: 5-(2-Aminopropoxy)-N-(1-(isoquinolin-5-yl)cyclopropyl)-2-methylbenzamide (Compound 446)

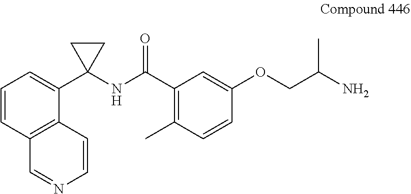 [SMILES: Cc1ccc(OCC(C)N)cc1C(=O)NC1(c2cccc3cnccc23)CC1]

Compound 446 was synthesized according to an analogous procedure to the one described for compound 445. M+H⁺=376.2 (LCMS); ¹H NMR (400 MHZ, DMSO-d₆) δ 9.63 (s, 1H), 9.25 (s, 1H), 8.68-8.82 (m, 2H), 8.25 (dd, J=7.69, 4.06 Hz, 2H), 7.97-8.11 (m, 3H), 7.83 (t, J=7.75 Hz, 1H), 7.08 (d, J=8.51 Hz, 1H), 6.90 (dd, J=8.38, 2.63 Hz, 1H), 6.70 (d, J=2.75 Hz, 1H), 4.03 (dd, J=10.26, 3.88 Hz, 1H), 3.87 (dd, J=10.19, 7.07 Hz, 1H), 3.48-3.62 (m, 1H), 1.94 (s, 3H), 1.39 (br s, 2H), 1.19-1.28 (m, 5H).

### Example 486: (S)—N-(3-(7-Methoxyquinolin-5-yl)oxetan-3-yl)-2-methyl-5-((1-methylazetidin-2-yl)methoxy)benzamide (Compound 566)

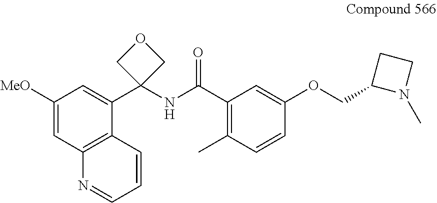 [SMILES: COc1cc(C2(NC(=O)c3cc(OC[C@@H]4CCN4C)ccc3C)COC2)c2cccnc2c1]

Compound 566 was synthesized according to an analogous procedure to the one described for compound 450. M+H⁺=448.2 (LCMS); ¹H NMR (400 MHZ, CD₃OD) δ 8.78 (dd, J=1.5, 4.4 Hz, 1H), 8.09 (d, J=8.1 Hz, 1H), 7.56 (d, J=2.5 Hz, 1H), 7.43-7.32 (m, 2H), 7.06 (d, J=8.5 Hz, 1H), 6.87 (dd, J=2.6, 8.4 Hz, 1H), 6.74 (d, J=2.8 Hz, 1H), 5.36-5.28 (m, 4H), 4.00 (s, 3H), 3.93 (d, J=5.5 Hz, 2H), 3.56-3.46 (m, 1H), 3.41 (dt, J=2.8, 7.8 Hz, 1H), 3.03-2.89 (m, 1H), 2.39 (s, 3H), 2.15-2.01 (m, 2H), 1.98 (s, 3H).

### Example 487: 5-(3-Aminoazetidin-1-yl)-2-methyl-N-(1-(naphthalen-1-yl)cyclopropyl)benzamide (Compound 464)

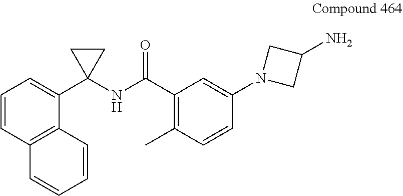 [SMILES: Cc1ccc(N2CC(N)C2)cc1C(=O)NC1(c2cccc3ccccc23)CC1]

Compound 464 was synthesized according to an analogous procedure to the one described for compound 463. M+H⁺=372.1 (LCMS); ¹H NMR (400 MHz, DMSO-d₆) δ 9.02 (s, 1H), 8.65 (d, J=8.4 Hz, 1H), 8.26 (br s, 3H), 7.98-7.89 (m, 1H), 7.86-7.77 (m, 2H), 7.60-7.42 (m, 3H), 6.95 (d, J=8.1 Hz, 1H), 6.41 (dd, J=2.4, 8.2 Hz, 1H), 6.18 (d, J=2.5 Hz, 1H), 4.09-4.02 (m, 1H), 3.97 (t, J=7.7 Hz, 2H), 3.68-3.65 (m, 2H), 1.91 (s, 3H), 1.40-1.31 (m, 2H), 1.23-1.13 (m, 2H).

### Example 488: 5-(3-Aminopyrrolidin-1-yl)-2-methyl-N-(1-(naphthalen-1-yl)cyclopropyl)benzamide (Compound 485)

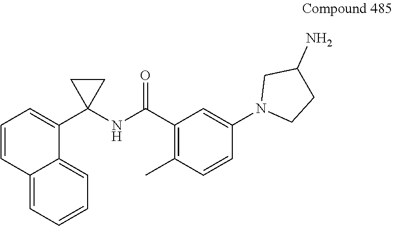 [SMILES: Cc1ccc(N2CCC(N)C2)cc1C(=O)NC1(c2cccc3ccccc23)CC1]

Compound 485 was synthesized according to an analogous procedure to the one described for compound 463. M+H⁺=386.1 (LCMS); ¹H NMR (400 MHZ, DMSO-d₆) δ 9.01 (s, 1H), 8.67 (d, J=8.3 Hz, 1H), 8.16 (br s, 3H), 7.93 (d, J=7.9 Hz, 1H), 7.85-7.78 (m, 2H), 7.61-7.43 (m, 3H), 6.94 (d, J=8.4 Hz, 1H), 6.48 (dd, J=2.6, 8.3 Hz, 1H), 6.25 (d, J=2.5 Hz, 1H), 3. 46-3.29 (m, 2H), 3.22-3.12 (m, 2H), 2.58-2.52 (m, 1H), 2.34-2.21 (m, 1H), 2.07-1.96 (m, 1H), 1.91 (s, 3H), 1.35 (s, 2H), 1.22-1.13 (m, 2H).

### Example 489: 5-((3S,5R)-3,5-Dimethylpiperazin-1-yl)-2-methyl-N-(1-(naphthalen-1-yl)cyclopropyl)benzamide (Compound 506)

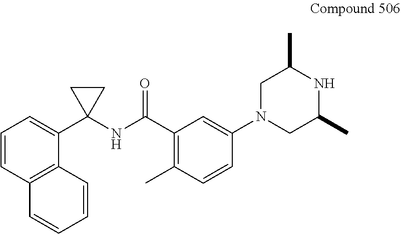 [SMILES: Cc1ccc(N2C[C@@H](C)N[C@@H](C)C2)cc1C(=O)NC1(c2cccc3ccccc23)CC1]

Compound 506 was synthesized according to an analogous procedure to the one described for compound 463. M+H⁺=414.1 (LCMS); ¹H NMR (400 MHZ, DMSO-d₆) δ 9.54-9.30 (m, 1H), 9.05 (s, 1H), 8.91-8.63 (m, 2H), 7.93 (d, J=7.5 Hz, 1H), 7.82 (dd, J=7.6, 10.5 Hz, 2H), 7.60-7.41 (m, 3H), 7.03-6.97 (m, 1H), 6.91 (s, 1H), 6.64 (d, J=2.5 Hz, 1H), 3.67 (br d, J=11.4 Hz, 2H), 3.27 (br s, 2H), 2.56 (br t, J=11.9 Hz, 2H), 1.92 (s, 3H), 1.36 (s, 2H), 1.26 (d, J=6.4 Hz, 6H), 1.18 (br s, 2H).

### Example 490: 5-(2-Aminopropoxy)-2-methyl-N-(1-(3-(5-methylthiophen-2-yl)naphthalen-1-yl)cyclopropyl)benzamide (Compound 479)

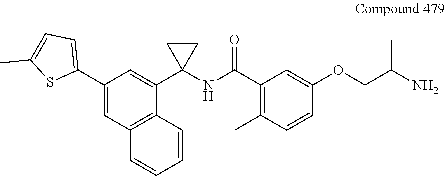 [SMILES: Cc1ccc(-c2cc(C3(NC(=O)c4cc(OCC(C)N)ccc4C)CC3)c3ccccc3c2)s1]

Compound 479 was synthesized according to an analogous procedure to the one described for compound 470. M+H⁺=471.2 (LCMS); ¹H NMR (400 MHZ, CD₃OD) δ 9.24 (s, 1H), 8.56-8.49 (m, 1H), 8.18 (d, J=1.88 Hz, 1H), 7.94 (d, J=1.50 Hz, 1H), 7.92-7.87 (m, 1H), 7.55-7.46 (m, 2H), 7.37 (d, J=3.50 Hz, 1H), 7.08 (d, J=8.50 Hz, 1H), 6.90 (dd, J=8.50, 2.75 Hz, 1H), 6.81 (dd, J=3.56, 1.06 Hz, 1H), 6.71 (d, J=2.63 Hz, 1H), 4.07 (dd, J=10.32, 3.56 Hz, 1H), 3.86 (dd, J=10.38, 7.25 Hz, 1H), 3.62 (td, J=6.88, 3.50 Hz, 1H), 2.53 (s, 3H), 2.04 (s, 3H), 1.47 (s, 2H), 1.39-1.30 (m, 5H).

### Example 491: 5-(2-Aminopropoxy)-N-(1-(3-(5-chlorothiophen-2-yl)naphthalen-1-yl)cyclopropyl)-2-methylbenzamide (Compound 502)

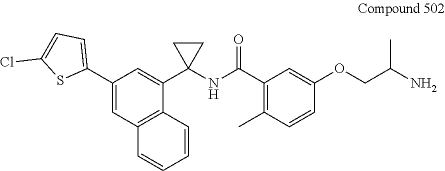 [SMILES: Cc1ccc(OCC(C)N)cc1C(=O)NC1(c2cc(-c3ccc(Cl)s3)cc3ccccc23)CC1]

Compound 502 was synthesized according to an analogous procedure to the one described for compound 470. M+H⁺=491.2 (LCMS); ¹H NMR (400 MHZ, DMSO-d₆) δ 9.12 (s, 1H), 8.67-8.59 (m, 1H), 8.06 (d, J=1.50 Hz, 1H), 8.02-7.95 (m, 2H), 7.61-7.51 (m, 3H), 7.23 (d, J=3.88 Hz, 1H), 7.02 (d, J=8.50 Hz, 1H), 6.83 (dd, J=8.32, 2.69 Hz, 1H), 6.60 (d, J=2.63 Hz, 1H), 3.67-3.56 (m, 2H), 3.11-3.00 (m, 1H), 1.96 (s, 3H), 1.38 (br s, 2H), 1.30-1.23 (m, 2H), 1.00 (d, J=6.50 Hz, 3H).

### Example 492: 5-(2-Aminopropoxy)-N-(1-(3-(4-fluorophenyl)naphthalen-1-yl)cyclopropyl)-2-methylbenzamide (Compound 480)

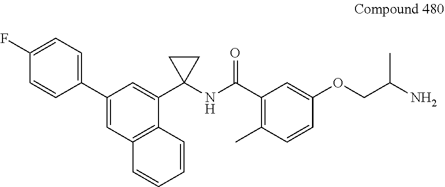 [SMILES: Cc1ccc(OCC(C)N)cc1C(=O)NC1(c2cc(-c3ccc(F)cc3)cc3ccccc23)CC1]

Compound 480 was synthesized according to an analogous procedure to the one described for compound 470. M+H⁺=469.3 (LCMS); ¹H NMR (400 MHZ, CD₃OD) δ 8.60 (d, J=8.13 Hz, 1H), 8.23 (d, J=1.88 Hz, 1H), 8.06 (d, J=1.38 Hz, 1H), 8.03-7.97 (m, 1H), 7.85 (dd, J=8.75, 5.38 Hz, 2H), 7.65-7.53 (m, 2H), 7.25 (t, J=8.82 Hz, 2H), 7.11 (d, J=8.50 Hz, 1H), 6.94 (br d, J=2.75 Hz, 1H), 6.73 (d, J=2.63 Hz, 1H), 4.10 (dd, J=10.26, 3.50 Hz, 1H), 3.88 (s, 1H), 3.70-3.59 (m, 1H), 2.05 (s, 3H), 1.50 (br s, 2H), 1.44-1.38 (m, 2H), 1.36 (d, J=6.75 Hz, 3H).

### Example 493: N-(1-(3-Ethoxynaphthalen-1-yl)cyclopropyl)-2-methyl-5-((1-methylazetidin-2-yl)methoxy)benzamide (Compound 478)

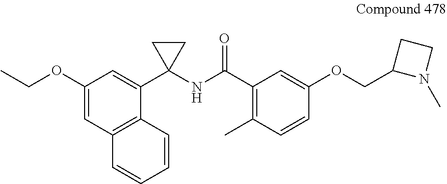 [SMILES: CCOc1cc(C2(NC(=O)c3cc(OCC4CCN4C)ccc3C)CC2)c2ccccc2c1]

Compound 478 was synthesized according to an analogous procedure to the one described for compound 474. M+H⁺=445.1 (LCMS); ¹H NMR (400 MHZ, DMSO-d₆) δ 9.11-9.04 (m, 1H), 8.57-8.50 (m, 1H), 8.22-8.15 (m, 1H), 7.86-7.77 (m, 1H), 7.48-7.33 (m, 3H), 7.25-7.17 (m, 1H), 7.07-6.98 (m, 1H), 6.88-6.78 (m, 1H), 6.67-6.52 (m, 1H), 4.18-4.09 (m, 2H), 3.90-3.84 (m, 2H), 3.30-3.24 (m, 2H), 2.76 (br d, J=7.8 Hz, 1H), 2.23 (s, 3H), 2.03-1.78 (m, 5H), 1.44-1.38 (m, 3H), 1.35-1.29 (m, 2H), 1.19-1.12 (m, 2H).

### Example 494: N-(1-(3-Isopropoxynaphthalen-1-yl)cyclopropyl)-2-methyl-5-((1-methyl azetidin-2-yl)methoxy)benzamide (Compound 476)

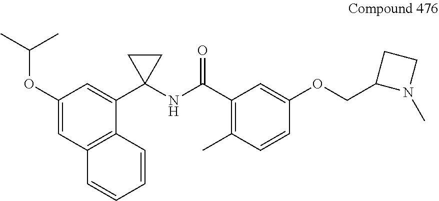 [SMILES: Cc1ccc(OCC2CCN2C)cc1C(=O)NC1(c2cc(OC(C)C)cc3ccccc23)CC1]

Compound 476 was synthesized according to an analogous procedure to the one described for compound 474. M+H⁺=459.1 (LCMS); ¹H NMR (400 MHZ, DMSO-d₆) δ 9.09-9.03 (m, 1H), 8.56-8.49 (m, 1H), 8.22-8.17 (m, 1H), 7.83-7.76 (m, 1H), 7.47-7.33 (m, 3H), 7.24-7.19 (m, 1H), 7.05-6.99 (m, 1H), 6.85-6.80 (m, 1H), 6.62-6.57 (m, 1H), 4.82-4.68 (m, 1H), 3.91-3.82 (m, 2H), 3.27-3.20 (m, 2H), 2.77-2.70 (m, 1H), 2.23-2.19 (m, 3H), 2.03-1.78 (m, 5H), 1.37-1.33 (m, 6H), 1.33-1.30 (m, 2H), 1.18-1.12 (m, 2H).

### Example 495: (S)-2-Methyl-5-((1-methylazetidin-2-yl)methoxy)-N-(1-(7-(neopentyloxy)quinolin-5-yl)cyclopropyl)benzamide (Compound 787)

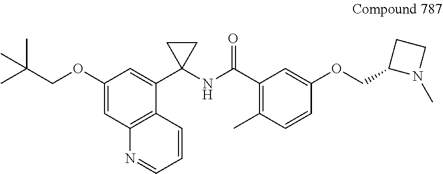 [SMILES: Cc1ccc(OC[C@@H]2CCN2C)cc1C(=O)NC1(c2cc(OCC(C)(C)C)cc3ncccc23)CC1]

Compound 787 was synthesized according to an analogous procedure to the one described for compound 474. M+H⁺=488.3 (LCMS); ¹H NMR (400 MHZ, DMSO-d₆) δ 9.67-9.59 (m, 1H), 9.34-9.28 (m, 1H), 9.12-9.05 (m, 1H), 7.97-7.88 (m, 1H), 7.77-7.71 (m, 1H), 7.53-7.46 (m, 1H), 7.13-7.06 (m, 1H), 6.95-6.89 (m, 1H), 6.76-6.71 (m, 1H), 4.66-4.56 (m, 1H), 4.31-4.16 (m, 2H), 4.05-3.98 (m, 1H), 3.92-3.85 (m, 3H), 2.86-2.67 (m, 3H), 2.43-2.26 (m, 2H), 1.96-1.90 (m, 3H), 1.46-1.39 (m, 2H), 1.35-1.27 (m, 2H), 1.09-1.02 (m, 9H).

### Example 496: 2-Methyl-5-((1-methylazetidin-2-yl)methoxy)-N-(1-(3-(5-methylthiophen-2-yl)naphthalen-1-yl)cyclopropyl)benzamide (Compound 493)

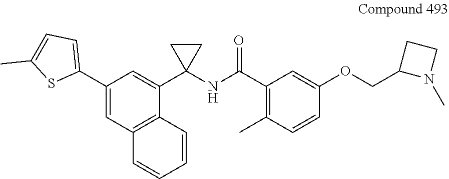

Compound 493 was synthesized according to an analogous procedure to the one described for compound 475. M+H⁺=497.1 (LCMS); ¹H NMR (400 MHZ, DMSO-d₆) δ 9.91 (br s, 1H), 9.13 (s, 1H), 8.64-8.52 (m, 1H), 8.04-7.98 (m, 2H), 7.97-7.93 (m, 1H), 7.57-7.49 (m, 2H), 7.44 (d, J=3.5 Hz, 1H), 7.08 (d, J=8.3 Hz, 1H), 6.94-6.85 (m, 2H), 6.69 (d, J=2.6 Hz, 1H), 4.63-4.51 (m, 1H), 4.27-4.17 (m, 2H), 4.05-3.96 (m, 1H), 3.84 (br dd, J=6.5, 9.4 Hz, 1H), 2.82 (d, J=4.8 Hz, 3H), 2.50 (br s, 3H), 2.43-2.28 (m, 2H), 1.98 (s, 3H), 1.36 (br s, 2H), 1.25 (br s, 2H).

### Example 497: N-(1-(3-(5-Chlorothiophen-2-yl)naphthalen-1-yl)cyclopropyl)-2-methyl-5-((1-methylazetidin-2-yl)methoxy)benzamide (Compound 514)

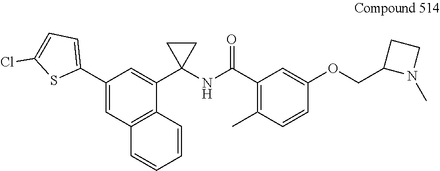 [SMILES: Cc1ccc(OCC2CCN2C)cc1C(=O)NC1(c2cc(-c3ccc(Cl)s3)cc3ccccc23)CC1]

Compound 514 was synthesized according to an analogous procedure to the one described for compound 475. M+H⁺=517.0 (LCMS); ¹H NMR (400 MHZ, DMSO-d₆) δ 10.38-10.11 (m, 1H), 9.15 (s, 1H), 8.66-8.61 (m, 1H), 8.07 (d, J=1.5 Hz, 1H), 8.03-7.98 (m, 2H), 7.59-7.54 (m, 3H), 7.24 (d, J=4.0 Hz, 1H), 7.08 (s, 1H), 6.93-6.87 (m, 1H), 6.70 (d, J=2.8 Hz, 1H), 4.68-4.56 (m, 1H), 4.32-4.18 (m, 2H), 4.06-3.95 (m, 1H), 3.90-3.76 (m, 1H), 2.82 (d, J=5.1 Hz, 3H), 2.38-2.28 (m, 2H), 1.98 (s, 3H), 1.38 (br s, 2H), 1.28 (br s, 2H).

### Example 498: N-(1-(3-(4-Fluorophenyl)naphthalen-1-yl)cyclopropyl)-2-methyl-5-((1-methylazetidin-2-yl)methoxy)benzamide (Compound 518)

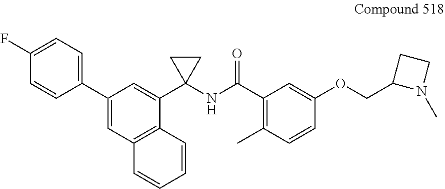 [SMILES: Cc1ccc(OCC2CCN2C)cc1C(=O)NC1(c2cc(-c3ccc(F)cc3)cc3ccccc23)CC1]

Compound 518 was synthesized according to an analogous procedure to the one described for compound 475. M+H⁺=495.1 (LCMS); ¹H NMR (400 MHZ, DMSO-d₆) δ 9.13 (s, 1H), 8.66 (d, J=7.9 Hz, 1H), 8.24-8.16 (m, 1H), 8.14-8.08 (m, 2H), 8.01 (br d, J=7.5 Hz, 1H), 7.87 (dd, J=5.9, 7.9 Hz, 2H), 7.65-7.50 (m, 2H), 7.37 (s, 2H), 7.08-6.97 (m, 1H), 6.89-6.76 (m, 1H), 6.60 (d, J=2.4 Hz, 1H), 3.86 (d, J=5.0 Hz, 2H), 3.26 (br d, J=1.1 Hz, 2H), 2.78-2.68 (m, 1H), 2.22 (s, 3H), 1.98 (s, 3H), 1.94 (br s, 2H), 1.38 (br s, 2H), 1.29 (br d, J=2.6 Hz, 2H).

### Example 499: N-(1-(3-Acetamidonaphthalen-1-yl)cyclopropyl)-2-methyl-5-((1-methylazetidin-2-yl)methoxy)benzamide (Compound 500)

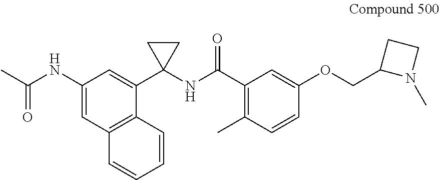 [SMILES: CC(=O)Nc1cc(C2(NC(=O)c3cc(OCC4CCN4C)ccc3C)CC2)c2ccccc2c1]

Compound 500 was synthesized according to an analogous procedure to the one described for compound 482. M+H⁺=458.2 (LCMS); ¹H NMR (400 MHz, DMSO-d₆) δ 10.20 (s, 1H), 9.93-9.80 (m, 1H), 9.12 (s, 1H), 8.58-8.50 (m, 1H), 8.35 (d, J=1.50 Hz, 1H), 7.85-7.77 (m, 2H), 7.50-7.39 (m, 2H), 7.10 (d, J=8.38 Hz, 1H), 6.91 (dd, J=8.38, 2.75 Hz, 1H), 6.76-6.68 (m, 1H), 4.60 (br d, J=3.63 Hz, 1H), 4.28-4.14 (m, 2H), 4.07-3.96 (m, 1H), 3.92-3.80 (m, 1H), 2.83 (d, J=4.13 Hz, 3H), 2.43-2.27 (m, 2H), 2.10 (s, 3H), 1.99 (s, 3H), 1.35 (br s, 2H), 1.16 (br s, 2H).

### Example 500: 5-(((2R,4S)-4-Fluoropyrrolidin-2-yl)methoxy)-N-(1-(3-methoxynaphthalen-1-yl)cyclopropyl)-2-methylbenzamide (Compound 510)

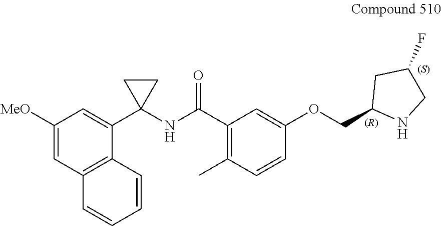

Compound 510 was synthesized according to an analogous procedure to the one described for compound 490. M+H⁺=449.1 (LCMS); ¹H NMR (400 MHZ, DMSO-d₆) δ 10.04 (br d, J=8.0 Hz, 1H), 9.73-9.48 (m, 1H), 9.13 (s, 1H), 8.55 (d, J=8.3 Hz, 1H), 7.83 (d, J=8.0 Hz, 1H), 7.53-7.34 (m, 3H), 7.24 (d, J=2.5 Hz, 1H), 7.08 (d, J=8.4 Hz, 1H), 6.90 (dd, J=2.8, 8.4 Hz, 1H), 6.68 (d, J=2.6 Hz, 1H), 5.62-5.30 (m, 1H), 4.27-4.20 (m, 1H), 4.19-4.10 (m, 1H), 4.09-3.98 (m, 1H), 3.87 (s, 3H), 3.50 (br s, 2H), 2.44-2.28 (m, 1H), 2.12-1.91 (m, 4H), 1.34 (br s, 2H), 1.16 (br s, 2H).

### Example 501: 5-(((2S,4R)-4-Fluoropyrrolidin-2-yl)methoxy)-N-(1-(7-methoxyquinolin-5-yl)cyclopropyl)-2-methylbenzamide (Compound 512)

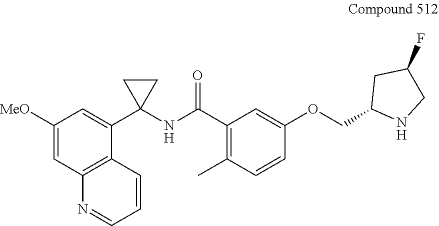 [SMILES: COc1cc(C2(NC(=O)c3cc(OC[C@@H]4C[C@@H](F)CN4)ccc3C)CC2)c2cccnc2c1]

Compound 512 was synthesized according to an analogous procedure to the one described for compound 490. M+H⁺=450.1 (LCMS); ¹H NMR (400 MHZ, DMSO-d₆) δ 10.26-10.11 (m, 1H), 9.88-9.68 (m, 2H), 9.44-9.36 (m, 1H), 9.22-9.14 (m, 1H), 8.02-7.94 (m, 1H), 7.76-7.69 (m, 2H), 7.09 (d, J=8.5 Hz, 1H), 6.92 (dd, J=2.6, 8.4 Hz, 1H), 6.76 (d, J=2.6 Hz, 1H), 5.60-5.32 (m, 1H), 4.29-4.16 (m, 3H), 4.02 (s, 3H), 3.63-3.40 (m, 2H), 2.45-2.29 (m, 1H), 2.00 (br s, 1H), 1.99-1.95 (m, 3H), 1.45-1.40 (m, 2H), 1.35-1.28 (m, 2H).

### Example 502: 5-(((2R,4S)-4-Fluoropyrrolidin-2-yl)methoxy)-N-(1-(7-methoxyquinolin-5-yl)cyclopropyl)-2-methylbenzamide (Compound 556)

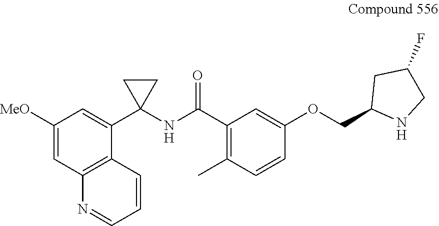 [SMILES: COc1cc(C2(NC(=O)c3cc(OC[C@H]4C[C@H](F)CN4)ccc3C)CC2)c2cccnc2c1]

Compound 556 was synthesized according to an analogous procedure to the one described for compound 490. M+H⁺=450.1 (LCMS); ¹H NMR (400 MHZ, DMSO-d₆) δ 9.09 (s, 1H), 9.01-8.88 (m, 1H), 8.81 (dd, J=1.6, 4.2 Hz, 1H), 7.51-7.37 (m, 2H), 7.31 (d, J=2.5 Hz, 1H), 7.03 (d, J=8.4 Hz, 1H), 6.83 (dd, J=2.6, 8.4 Hz, 1H), 6.61 (d, J=2.6 Hz, 1H), 5.34-5.10 (m, 1H), 3.92 (s, 3H), 3.82-3.68 (m, 2H), 3.60-3.48 (m, 1H), 3.02 (d, J=2.1 Hz, 1H), 2.97-2.90 (m, 1H), 2.15-1.98 (m, 1H), 1.94 (s, 3H), 1.77-1.52 (m, 1H), 1.42-1.29 (m, 2H), 1.21-1.12 (m, 2H).

### Example 503: 2-Methyl-5-((1-methylazetidin-2-yl)methoxy)-N-(1-(3-vinylnaphthalen-1-yl)cyclopropyl)benzamide (Compound 517)

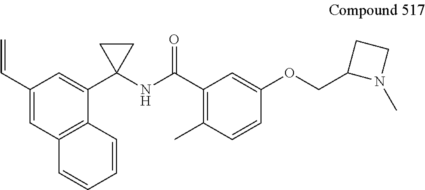 [SMILES: C=Cc1cc(C2(NC(=O)c3cc(OCC4CCN4C)ccc3C)CC2)c2ccccc2c1]

Compound 517 was synthesized according to an analogous procedure to the one described for compound 508. M+H⁺=427.1 (LCMS); ¹H NMR (400 MHZ, DMSO-d₆) δ 10.26-10.15 (m, 1H), 9.11 (s, 1H), 8.61 (d, J=7.5 Hz, 1H), 7.98 (d, J=1.6 Hz, 1H), 7.94-7.90 (m, 1H), 7.84 (s, 1H), 7.53 (dquin, J=1.4, 6.9 Hz, 2H), 7.11-7.07 (m, 1H), 6.96-6.87 (m, 2H), 6.69 (d, J=2.8 Hz, 1H), 5.98 (d, J=17.5 Hz, 1H), 5.39 (d, J=11.4 Hz, 1H), 4.69-4.51 (m, 1H), 4.32-4.16 (m, 2H), 4.08-3.94 (m, 1H), 3.90-3.77 (m, 1H), 2.82 (d, J=4.9 Hz, 3H), 2.75-2.59 (m, 1H), 2.38-2.26 (m, 2H), 1.98 (s, 3H), 1.36 (br s, 2H), 1.23 (br s, 2H).

### Example 504: N-(1-(3-Allylnaphthalen-1-yl)cyclopropyl)-2-methyl-5-((1-methylazetidin-2-yl)methoxy)benzamide (Compound 503)

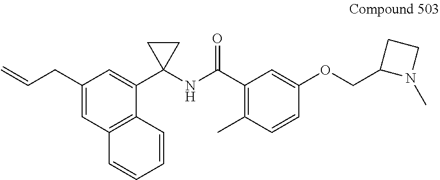 [SMILES: C=CCc1cc(C2(NC(=O)c3cc(OCC4CCN4C)ccc3C)CC2)c2ccccc2c1]

Compound 503 was synthesized according to an analogous procedure to the one described for compound 508. M+H⁺=441.2 (LCMS); ¹H NMR (400 MHZ, DMSO-d₆) δ 10.31-10.10 (m, 1H), 9.09 (s, 1H), 8.61 (br d, J=7.5 Hz, 1H), 7.90-7.84 (m, 1H), 7.68 (d, J=1.5 Hz, 1H), 7.61 (s, 1H), 7.54-7.46 (m, 2H), 7.09 (d, J=8.4 Hz, 1H), 6.90 (dd, J=2.6, 8.3 Hz, 1H), 6.69 (d, J=2.6 Hz, 1H), 6.06 (tdd, J=6.8, 10.1, 17.0 Hz, 1H), 5.23-5.07 (m, 2H), 4.65-4.54 (m, 1H), 4.32-4.16 (m, 2H), 4.05-3.94 (m, 1H), 3.90-3.79 (m, 1H), 3.54 (d, J=6.6 Hz, 2H), 2.81 (br s, 3H), 2.39-2.28 (m, 2H), 1.98 (s, 3H), 1.35 (br s, 2H), 1.21-1.12 (m, 2H).

### Example 505: (S)-2-Methyl-N-(1-(7-(1-methyl-1H-pyrazol-5-yl)quinolin-5-yl)cyclopropyl)-5-((1-methylazetidin-2-yl)methoxy)benzamide (Compound 578)

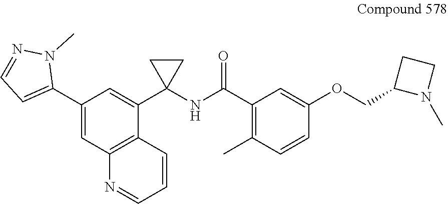 [SMILES: Cc1ccc(OC[C@@H]2CCN2C)cc1C(=O)NC1(c2cc(-c3ccnn3C)cc3ncccc23)CC1]

Compound 578 was synthesized according to an analogous procedure to the one described for compound 508. M+H⁺=482.1 (LCMS); ¹H NMR (400 MHZ, DMSO-d₆) δ 11.11 (br s, 1H), 9.76 (d, J=8.6 Hz, 1H), 9.55-9.41 (m, 1H), 9.32 (d, J=4.0 Hz, 1H), 8.50 (s, 1H), 8.28 (d, J=1.5 Hz, 1H), 8.12 (dd, J=5.1, 8.6 Hz, 1H), 7.63 (d, J=1.9 Hz, 1H), 7.15-7.05 (m, 1H), 6.98-6.89 (m, 1H), 6.83-6.73 (m, 2H), 4.68-4.59 (m, 1H), 4.44 (dd, J=8.3, 11.2 Hz, 1H), 4.26-4.19 (m, 1H), 4.08-4.00 (m, 5H), 2.85-2.65 (m, 3H), 2.38-2.25 (m, 2H), 2.01-1.95 (m, 3H), 1.47 (br s, 2H), 1.39 (br s, 2H).

### Example 506: (R)-5-(Azetidin-2-ylmethoxy)-N-(1-(7-methoxyquinolin-5-yl)cyclopropyl)-2-methylbenzamide (Compound 554)

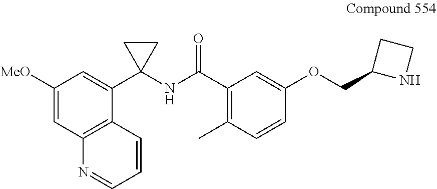 [SMILES: COc1cc(C2(NC(=O)c3cc(OC[C@H]4CCN4)ccc3C)CC2)c2cccnc2c1]

Compound 554 was synthesized according to an analogous procedure to the one described for compound 511. M+H⁺=418.1 (LCMS); ¹H NMR (400 MHZ, DMSO-d₆) δ 9.72 (d, J=8.4 Hz, 1H), 9.35 (s, 1H), 9.17 (dd, J=1.2, 5.4 Hz, 1H), 7.99 (dd, J=5.4, 8.4 Hz, 1H), 7.75 (d, J=2.4 Hz, 1H), 7.61 (d, J=2.3 Hz, 1H), 7.09 (d, J=8.5 Hz, 1H), 6.92 (dd, J=2.8, 8.4 Hz, 1H), 6.75 (d, J=2.8 Hz, 1H), 4.65 (dq, J=3.3, 7.9 Hz, 1H), 4.29 (dd, J=7.4, 11.2 Hz, 1H), 4.15 (dd, J=3.4, 11.3 Hz, 1H), 4.02 (s, 3H), 3.96-3.87 (m, 1H), 3.82 (dt, J=6.3, 9.9 Hz, 1H), 2.47-2.41 (m, 1H), 2.40-2.28 (m, 1H), 1.96 (s, 3H), 1.42 (br s, 2H), 1.36-1.26 (m, 2H).

### Example 507: N-(1-(3-Ethylnaphthalen-1-yl)cyclopropyl)-2-methyl-5-((1-methylazetidin-2-yl)methoxy)benzamide (Compound 516)

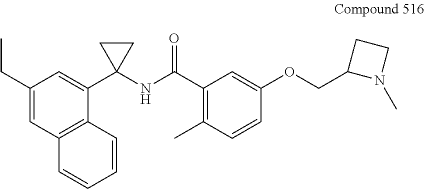 [SMILES: CCc1cc(C2(NC(=O)c3cc(OCC4CCN4C)ccc3C)CC2)c2ccccc2c1]

Compound 516 was synthesized according to an analogous procedure to the one described for compound 521. M+H⁺=429.1 (LCMS); ¹H NMR (400 MHZ, DMSO-d₆) § 10.56-10.43 (m, 1H), 9.09 (s, 1H), 8.65-8.53 (m, 1H), 7.89-7.82 (m, 1H), 7.69 (d, J=1.6 Hz, 1H), 7.61 (s, 1H), 7.52-7.44 (m, 2H), 7.08 (d, J=8.5 Hz, 1H), 6.95-6.86 (m, 1H), 6.69 (br s, 1H), 4.60 (br d, J=4.3 Hz, 1H), 4.36-4.26 (m, 1H), 4.24-4.16 (m, 1H), 3.98 (br s, 1H), 3.88-3.73 (m, 1H), 2.84-2.72 (m, 5H), 2.40-2.25 (m, 2H), 1.98 (s, 3H), 1.35 (br s, 2H), 1.32-1.25 (m, 3H), 1.22-1.14 (m, 2H).

### Example 508: 2-Methyl-5-((1-methylazetidin-2-yl)methoxy)-N-(1-(3-propylnaphthalen-1-yl)cyclopropyl)benzamide (Compound 504)

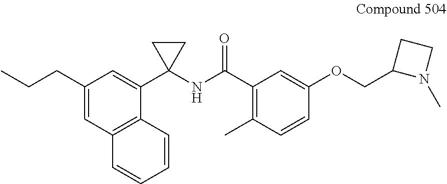 [SMILES: CCCc1cc(C2(NC(=O)c3cc(OCC4CCN4C)ccc3C)CC2)c2ccccc2c1]

Compound 504 was synthesized according to an analogous procedure to the one described for compound 521. M+H⁺=443.2 (LCMS); ¹H NMR (400 MHz, DMSO-d₆) δ 10.45-10.35 (m, 1H), 9.08 (s, 1H), 8.64-8.56 (m, 1H), 7.89-7.82 (m, 1H), 7.69 (d, J=1.6 Hz, 1H), 7.60 (s, 1H), 7.52-7.44 (m, 2H), 7.08 (d, J=8.5 Hz, 1H), 6.90 (dd, J=2.8, 8.4 Hz, 1H), 6.69 (d, J=2.6 Hz, 1H), 4.67-4.55 (m, 1H), 4.36-4.26 (m, 1H), 4.24-4.17 (m, 1H), 4.05-3.94 (m, 1H), 3.84 (br dd, J=6.5, 9.6 Hz, 1H), 2.81 (d, J=5.1 Hz, 3H), 2.75-2.66 (m, 2H), 2.40-2.26 (m, 2H), 2.02-1.95 (m, 3H), 1.75-1.63 (m, 2H), 1.35 (br s, 2H), 1.17 (br s, 2H), 0.95 (t, J=7.4 Hz, 3H).

### Example 509: (R)—N-(1-(7-Methoxyquinolin-5-yl)cyclopropyl)-2-methyl-5-((1-methylazetidin-2-yl)methoxy)benzamide (Compound 558)

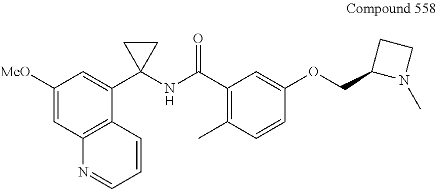 [SMILES: COc1cc(C2(NC(=O)c3cc(OC[C@H]4CCN4C)ccc3C)CC2)c2cccnc2c1]

Compound 558 was synthesized according to an analogous procedure to the one described for compound 527. M+H⁺=432.1 (LCMS); ¹H NMR (400 MHZ, DMSO-d₆) δ 9.64 (br d, J=8.6 Hz, 1H), 9.31 (s, 1H), 9.15 (d, J=5.3 Hz, 1H), 7.95 (dd, J=5.4, 8.5 Hz, 1H), 7.73 (d, J=2.5 Hz, 1H), 7.55 (s, 1H), 7.09 (d, J=8.5 Hz, 1H), 6.92 (dd, J=2.6, 8.4 Hz, 1H), 6.76 (d, J=2.8 Hz, 1H), 4.62 (dt, J=5.6, 8.0 Hz, 1H), 4.37-4.16 (m, 2H), 4.02 (s, 4H), 3.86 (d, J=9.6 Hz, 1H), 2.82 (s, 3H), 2.43-2.27 (m, 2H), 1.94 (s, 3H), 1.41 (br s, 2H), 1.31 (br s, 2H).

### Example 510: 5-(((2R,4S)-4-Fluoro-1-methylpyrrolidin-2-yl)methoxy)-N-(1-(3-methoxynaphthalen-1-yl)cyclopropyl)-2-methylbenzamide (Compound 531)

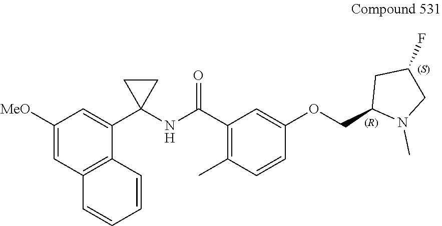

Compound 531 was synthesized according to an analogous procedure to the one described for compound 528. M+H⁺=463.1 (LCMS); ¹H NMR (400 MHZ, DMSO-d₆) δ 11.30-11.07 (m, 1H), 9.13 (s, 1H), 8.54 (d, J=8.3 Hz, 1H), 7.83 (d, J=7.9 Hz, 1H), 7.50-7.34 (m, 3H), 7.24 (d, J=2.4 Hz, 1H), 7.09 (d, J=8.5 Hz, 1H), 6.92 (dd, J=2.8, 8.4 Hz, 1H), 6.80-6.65 (m, 1H), 5.55-5.31 (m, 1H), 4.33 (br d, J=5.1 Hz, 2H), 4.10-3.98 (m, 2H), 3.91-3.87 (m, 3H), 3.55-3.38 (m, 1H), 2.97 (d, J=4.6 Hz, 3H), 2.49-2.37 (m, 1H), 2.22-2.03 (m, 1H), 1.97 (s, 3H), 1.34 (br s, 2H), 1.17 (br s, 2H).

### Example 511: 5-(((2S,4R)-4-Fluoro-1-methylpyrrolidin-2-yl)methoxy)-N-(1-(7-methoxyquinolin-5-yl)cyclopropyl)-2-methylbenzamide (Compound 532)

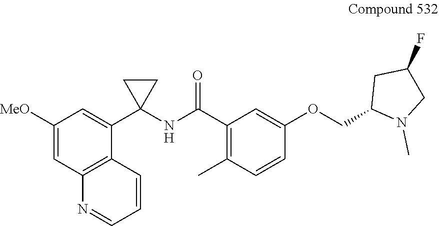 [SMILES: COc1cc(C2(NC(=O)c3cc(OC[C@@H]4C[C@@H](F)CN4C)ccc3C)CC2)c2cccnc2c1]

Compound 532 was synthesized according to an analogous procedure to the one described for compound 528. M+H⁺=464.1 (LCMS); ¹H NMR (400 MHZ, DMSO-d₆) δ 9.12-9.05 (m, 1H), 8.97-8.90 (m, 1H), 8.84-8.77 (m, 1H), 7.48-7.45 (m, 1H), 7.44-7.38 (m, 1H), 7.32-7.29 (m, 1H), 7.05-7.01 (m, 1H), 6.87-6.81 (m, 1H), 6.63-6.59 (m, 1H), 5.31-5.01 (m, 1H), 3.94-3.89 (m, 4H), 3.84-3.78 (m, 1H), 3.47-3.34 (m, 1H), 2.89-2.79 (m, 1H), 2.43-2.36 (m, 1H), 2.36-2.33 (m, 3H), 2.16-2.02 (m, 1H), 1.96-1.91 (m, 3H), 1.89-1.73 (m, 1H), 1.36-1.30 (m, 2H), 1.21-1.15 (m, 2H).

### Example 512: 5-(((2R,4S)-4-Fluoro-1-methylpyrrolidin-2-yl)methoxy)-N-(1-(7-methoxyquinolin-5-yl)cyclopropyl)-2-methylbenzamide (Compound 557)

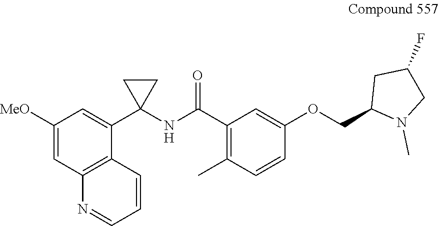 [SMILES: COc1cc(C2(NC(=O)c3cc(OC[C@H]4C[C@H](F)CN4C)ccc3C)CC2)c2cccnc2c1]

Compound 557 was synthesized according to an analogous procedure to the one described for compound 528. M+H⁺=464.1 (LCMS); ¹H NMR (400 MHZ, DMSO-d₆) δ 9.10 (s, 1H), 9.00-8.91 (m, 1H), 8.87-8.76 (m, 1H), 7.49-7.45 (m, 1H), 7.44-7.39 (m, 1H), 7.33-7.29 (m, 1H), 7.06-7.00 (m, 1H), 6.88-6.80 (m, 1H), 6.66-6.58 (m, 1H), 5.29-4.96 (m, 1H), 4.01-3.87 (m, 4H), 3.85-3.77 (m, 1H), 3.47-3.36 (m, 1H), 2.91-2.79 (m, 1H), 2.41 (br d, J=11.6 Hz, 1H), 2.34 (s, 3H), 2.18-1.99 (m, 1H), 1.94 (s, 3H), 1.91-1.70 (m, 1H), 1.34 (br s, 2H), 1.24-1.13 (m, 2H).

### Example 513: (S)—N-(1-(7-Methoxyquinolin-5-yl)cyclopropyl)-2-methyl-5-((1-methylpiperidin-2-yl)methoxy)benzamide (Compound 553)

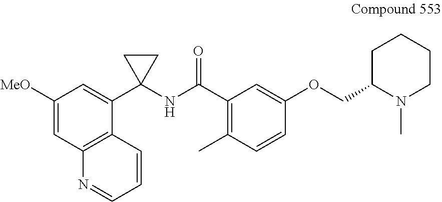 [SMILES: COc1cc(C2(NC(=O)c3cc(OC[C@@H]4CCCCN4C)ccc3C)CC2)c2cccnc2c1]

Compound 553 was synthesized according to an analogous procedure to the one described for compound 528. M+H⁺=460.2 (LCMS); ¹H NMR (400 MHZ, DMSO-d₆) δ 9.69 (d, J=8.4 Hz, 1H), 9.37 (s, 1H), 9.21-9.14 (m, 1H), 7.98 (dd, J=5.4, 8.4 Hz, 1H), 7.73 (d, J=2.5 Hz, 1H), 7.62 (d, J=2.1 Hz, 1H), 7.09 (d, J=8.5 Hz, 1H), 6.97-6.88 (m, 1H), 6.81-6.74 (m, 1H), 4.27-4.13 (m, 2H), 4.02 (s, 3H), 3.35 (br s, 1H), 3.28-3.18 (m, 1H), 3.09-3.01 (m, 1H), 2.78-2.68 (m, 3H), 1.96 (s, 3H), 1.94-1.86 (m, 1H), 1.80-1.66 (m, 4H), 1.57-1.44 (m, 1H), 1.42 (br s, 2H), 1.31 (br s, 2H).

### Example 514: (R)-5-(2-Aminopropoxy)-N-(1-(7-methoxyquinolin-5-yl)cyclopropyl)-2-methylbenzamide (Compound 535)

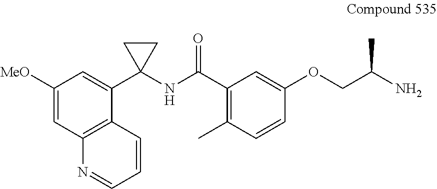 [SMILES: COc1cc(C2(NC(=O)c3cc(OC[C@@H](C)N)ccc3C)CC2)c2cccnc2c1]

Compound 535 was synthesized according to an analogous procedure to the one described for compound 530. M+H⁺=406.2 (LCMS); ¹H NMR (400 MHz, DMSO-d₆) δ 9.24-9.14 (m, 2H), 8.97 (br d, J=4.38 Hz, 1H), 7.95 (br s, 3H), 7.63 (br dd, J=8.19, 4.69 Hz, 1H), 7.57 (d, J=2.38 Hz, 1H), 7.39 (d, J=2.13 Hz, 1H), 7.09 (d, J=8.50 Hz, 1H), 6.90 (dd, J=8.38, 2.75 Hz, 1H), 6.69 (d, J=2.63 Hz, 1H), 4.03 (dd, J=10.26, 3.88 Hz, 1H), 3.96 (s, 3H), 3.86 (dd, J=10.26, 7.25 Hz, 1H), 3.61-3.48 (m, 1H), 1.95 (s, 3H), 1.36 (br s, 2H), 1.29-1.17 (m, 5H).

### Example 515: (S)-5-(2-Aminopropoxy)-N-(1-(7-methoxyisoquinolin-5-yl)cyclopropyl)-2-methylbenzamide (Compound 536)

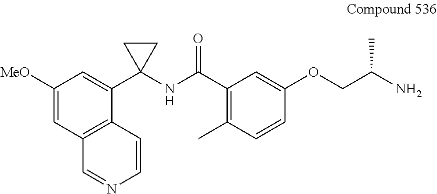 [SMILES: COc1cc(C2(NC(=O)c3cc(OC[C@H](C)N)ccc3C)CC2)c2ccncc2c1]

Compound 536 was synthesized according to an analogous procedure to the one described for compound 530. M+H⁺=406.1 (LCMS); ¹H NMR (400 MHZ, DMSO-d₆) δ 9.45 (s, 1H), 9.22 (s, 1H), 8.63 (d, J=6.13 Hz, 1H), 8.55 (d, J=6.13 Hz, 1H), 7.96 (br s, 3H), 7.80 (d, J=2.63 Hz, 1H), 7.63 (d, J=2.25 Hz, 1H), 7.08 (d, J=8.50 Hz, 1H), 6.89 (dd, J=8.44, 2.69 Hz, 1H), 6.69 (d, J=2.63 Hz, 1H), 4.02 (br d, J=6.38 Hz, 1H), 3.95 (s, 3H), 3.85 (br d, J=3.13 Hz, 1H), 3.58-3.51 (m, 1H), 1.94 (s, 3H), 1.40-1.31 (m, 2H), 1.22 (br d, J=6.75 Hz, 5H).

### Example 516: (R)-5-(2-Aminopropoxy)-N-(1-(7-methoxyisoquinolin-5-yl)cyclopropyl)-2-methylbenzamide (Compound 537)

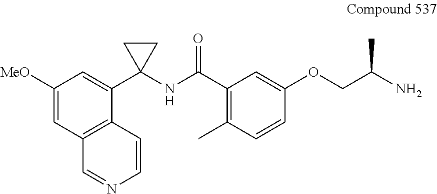 [SMILES: COc1cc(C2(NC(=O)c3cc(OC[C@@H](C)N)ccc3C)CC2)c2ccncc2c1]

Compound 537 was synthesized according to an analogous procedure to the one described for compound 530. M+H⁺=406.1 (LCMS); ¹H NMR (400 MHZ, DMSO-d₆) δ 9.50 (s, 1H), 9.23 (s, 1H), 8.69 (d, J=6.13 Hz, 1H), 8.59 (d, J=6.25 Hz, 1H), 7.95 (br s, 3H), 7.84 (d, J=2.38 Hz, 1H), 7.68 (d, J=2.25 Hz, 1H), 7.09 (d, J=8.51 Hz, 1H), 6.90 (dd, J=8.38, 2.63 Hz, 1H), 6.70 (d, J=2.63 Hz, 1H), 4.05-4.02 (m, 1H), 3.97 (s, 3H), 3.88-3.84 (m, 1H), 3.56 (br dd, J=10.57, 5.69 Hz, 1H), 1.95 (s, 3H), 1.37 (br s, 2H), 1.28-1.19 (m, 5H).

### Example 517: (R)-5-(2-Aminopropoxy)-2-methyl-N-(1-(3-(thiophen-2-yl)naphthalen-1-yl)cyclopropyl)benzamide (Compound 543)

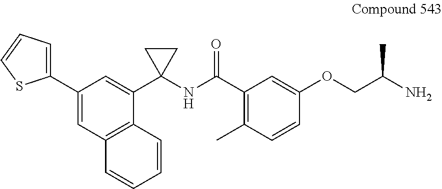 [SMILES: Cc1ccc(OC[C@@H](C)N)cc1C(=O)NC1(c2cc(-c3cccs3)cc3ccccc23)CC1]

Compound 543 was synthesized according to an analogous procedure to the one described for compound 539. M+H⁺=457.2 (LCMS); ¹H NMR (400 MHZ, DMSO-d₆) δ 9.21 (s, 1H), 8.71-8.59 (m, 1H), 8.16 (br s, 2H), 8.14-8.11 (m, 2H), 8.05-7.98 (m, 1H), 7.68 (dd, J=3.61, 0.92 Hz, 1H), 7.64 (dd, J=5.07, 0.92 Hz, 1H), 7.62-7.53 (m, 2H), 7.23 (dd, J=5.07, 3.61 Hz, 1H), 7.10 (d, J=8.44 Hz, 1H), 6.91 (dd, J=8.37, 2.63 Hz, 1H), 6.70 (d, J=2.57 Hz, 1H), 4.05 (dd, J=10.27, 3.91 Hz, 1H), 3.91 (dd, J=10.21, 7.03 Hz, 1H), 3.61-3.49 (m, 1H), 2.01 (s, 3H), 1.44-1.38 (m, 2H), 1.32-1.22 (m, 5H).

### Example 518: (R)-5-(2-Aminopropoxy)-N-(1-(3-bromonaphthalen-1-yl)cyclopropyl)-2-methylbenzamide (Compound 540)

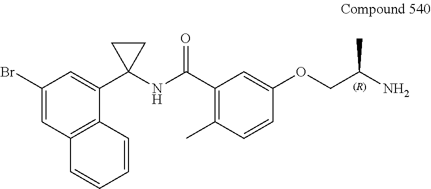

Compound 540 was synthesized according to an analogous procedure to the one described for compound 541. M+H⁺=453.0 (LCMS); ¹H NMR (400 MHZ, DMSO-d₆) δ 9.22 (s, 1H), 8.64 (d, J=8.25 Hz, 1H), 8.18 (br s, 2H), 8.15 (d, J=1.38 Hz, 1H), 7.94 (d, J=8.00 Hz, 1H), 7.88 (d, J=1.88 Hz, 1H), 7.60 (dt, J=19.76, 7.00 Hz, 2H), 7.08 (d, J=8.38 Hz, 1H), 6.89 (dd, J=8.38, 2.63 Hz, 1H), 6.68 (d, J=2.50 Hz, 1H), 4.04 (dd, J=10.26, 4.00 Hz, 1H), 3.90 (dd, J=10.13, 7.13 Hz, 1H), 3.52 (br d, J=4.75 Hz, 1H), 1.96 (s, 3H), 1.36 (br s, 2H), 1.29-1.18 (m, 5H).

### Example 519: N-(3-Fluorobenzyl)-1-(1-(2-isopropylphenyl)ethyl)piperidine-4-carboxamide (Compound 552)

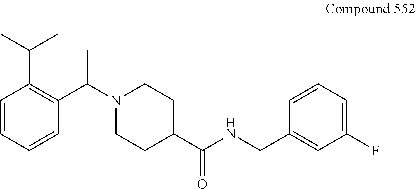 [SMILES: CC(C)c1ccccc1C(C)N1CCC(C(=O)NCc2cccc(F)c2)CC1]

Compound 552 was synthesized according to an analogous procedure to the one described for compound 549. M+H⁺=383.1 (LCMS); ¹H NMR (400 MHZ, DMSO-d₆) δ 9.80 (br dd, J=1.8, 4.9 Hz, 1H), 8.62-8.48 (m, 1H), 7.83-7.71 (m, 1H), 7.53-7.26 (m, 4H), 7.12-6.94 (m, 3H), 4.72 (br t, J=7.1 Hz, 1H), 4.33-4.22 (m, 2H), 3.90 (br d, J=10.5 Hz, 1H), 3.39-3.35 (m, 1H), 3.21-3.08 (m, 1H), 2.95 (br d, J=11.4 Hz, 1H), 2.82-2.69 (m, 1H), 2.48-2.40 (m, 1H), 2.16-1.96 (m, 2H), 1.93-1.71 (m, 2H), 1.65-1.60 (m, 3H), 1.28-1.22 (m, 3H), 1.11 (d, J=6.8 Hz, 3H).

### Example 520: N-(3-Fluorobenzyl)-1-(1-(7-methoxyquinolin-5-yl)ethyl)piperidine-4-carboxamide (Compound 593)

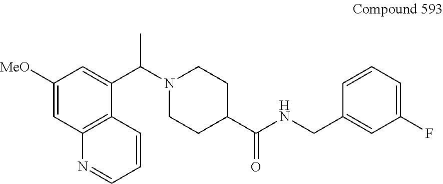 [SMILES: COc1cc(C(C)N2CCC(C(=O)NCc3cccc(F)c3)CC2)c2cccnc2c1]

Compound 593 was synthesized according to an analogous procedure to the one described for compound 549. M+H⁺=422.1 (LCMS); ¹H NMR (400 MHZ, DMSO-d₆) δ 8.83-8.70 (m, 2H), 8.29 (br t, J=5.9 Hz, 1H), 7.39-7.30 (m, 2H), 7.27 (d, J=2.3 Hz, 1H), 7.21 (d, J=2.3 Hz, 1H), 7.08-6.95 (m, 3H), 4.24 (d, J=5.4 Hz, 2H), 4.12 (q, J=6.4 Hz, 1H), 3.90 (s, 3H), 2.98 (br d, J=10.6 Hz, 1H), 2.78 (br d, J=11.1 Hz, 1H), 2.20-2.10 (m, 1H), 2.07-1.95 (m, 2H), 1.74-1.59 (m, 2H), 1.58-1.44 (m, 2H), 1.37 (d, J=6.6 Hz, 3H).

### Example 521: N-(3-Fluorobenzyl)-1-(1-(3-(thiophen-2-yl)naphthalen-1-yl)ethyl)piperidine-4-carboxamide (Compound 599)

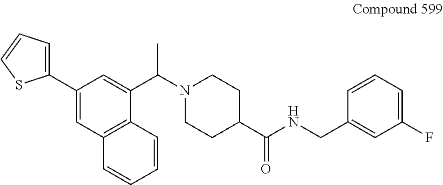 [SMILES: CC(c1cc(-c2cccs2)cc2ccccc12)N1CCC(C(=O)NCc2cccc(F)c2)CC1]

Compound 599 was synthesized according to an analogous procedure to the one described for compound 549. M+H⁺=473.2 (LCMS); ¹H NMR (400 MHZ, DMSO-d₆) δ 10.03-9.87 (m, 1H), 8.51 (br t, J=5.8 Hz, 1H), 8.45-8.33 (m, 2H), 8.27 (s, 1H), 8.11-8.05 (m, 1H), 7.82 (d, J=2.6 Hz, 1H), 7.68-7.57 (m, 3H), 7.40-7.31 (m, 1H), 7.26-7.20 (m, 1H), 7.11-6.98 (m, 3H), 5.46-5.28 (m, 1H), 4.31-4.24 (m, 2H), 4.02 (br d, J=12.1 Hz, 1H), 3.29-3.14 (m, 1H), 3.11-3.00 (m, 1H), 2.96-2.85 (m, 1H), 2.47-2.40 (m, 1H), 2.13-2.01 (m, 2H), 1.85-1.77 (m, 5H).

### Example 522: (R)—N-(1-(7-Methoxyquinolin-5-yl)cyclopropyl)-2-methyl-5-(2-(methylamino)propoxy)benzamide (Compound 573)

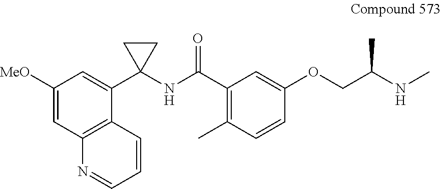 [SMILES: CN[C@H](C)COc1ccc(C)c(C(=O)NC2(c3cc(OC)cc4ncccc34)CC2)c1]

Compound 573 was synthesized according to an analogous procedure to the one described for compound 560. M+H⁺=420.2 (LCMS); ¹H NMR (400 MHZ, DMSO-d₆) δ 9.59-9.49 (m, 1H), 9.35-9.28 (m, 1H), 9.15-9.00 (m, 2H), 8.98-8.87 (m, 1H), 7.94-7.84 (m, 1H), 7.69 (br s, 1H), 7.62-7.54 (m, 1H), 7.09 (d, J=8.50 Hz, 1H), 6.92 (dd, J=8.38, 2.63 Hz, 1H), 6.74 (s, 1H), 4.16-4.11 (m, 1H), 4.07-4.03 (m, 1H), 4.01 (s, 3H), 3.53-3.46 (m, 1H), 2.58-2.52 (m, 3H), 1.96 (s, 3H), 1.44-1.36 (m, 2H), 1.33-1.24 (m, 5H).

### Example 523: N-(1-(7-Methoxyquinolin-5-yl)cyclopropyl)-2-methyl-5-(pyridin-2-ylmethoxy)benzamide (Compound 562)

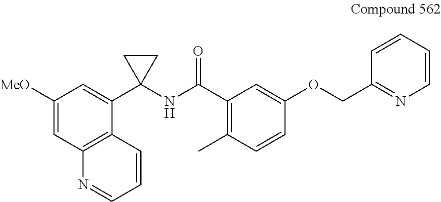 [SMILES: COc1cc(C2(NC(=O)c3cc(OCc4ccccn4)ccc3C)CC2)c2cccnc2c1]

Compound 562 was synthesized according to an analogous procedure to the one described for compound 561. M+H⁺=440.1 (LCMS); ¹H NMR (400 MHZ, DMSO-d₆) δ 9.69 (d, J=8.4 Hz, 1H), 9.33 (s, 1H), 9.20 (dd, J=1.3, 5.4 Hz, 1H), 8.68 (d, J=4.5 Hz, 1H), 8.11 (br t, J=7.9 Hz, 1H), 7.99 (dd, J=5.4, 8.4 Hz, 1H), 7.79-7.64 (m, 3H), 7.64-7.55 (m, 1H), 7.09 (d, J=8.6 Hz, 1H), 6.97 (dd, J=2.8, 8.4 Hz, 1H), 6.81 (d, J=2.8 Hz, 1H), 5.26 (s, 2H), 4.02 (s, 3H), 1.98 (s, 3H), 1.46-1.39 (m, 2H), 1.35-1.29 (m, 2H).

### Example 524: (S)-2-Methyl-5-((1-methylazetidin-2-yl)methoxy)-N-(1-(7-(trifluoromethoxy)quinolin-5-yl)cyclopropyl)benzamide (Compound 579)

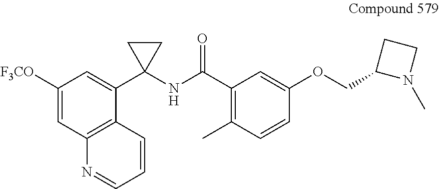 [SMILES: Cc1ccc(OC[C@@H]2CCN2C)cc1C(=O)NC1(c2cc(OC(F)(F)F)cc3ncccc23)CC1]

Compound 579 was synthesized according to an analogous procedure to the one described for compound 563. M+H⁺=486.1 (LCMS); ¹H NMR (400 MHZ, DMSO-d₆) δ 10.36-10.25 (m, 1H), 9.26 (s, 1H), 9.16 (d, J=8.1 Hz, 1H), 9.03 (dd, J=1.4, 4.2 Hz, 1H), 7.94-7.79 (m, 2H), 7.72 (dd, J=4.3, 8.5 Hz, 1H), 7.09 (d, J=8.5 Hz, 1H), 6.92 (dd, J=2.8, 8.4 Hz, 1H), 6.73 (d, J=2.6 Hz, 1H), 4.69-4.56 (m, 1H), 4.34-4.18 (m, 2H), 4.06-3.96 (m, 1H), 3.92-3.80 (m, 1H), 2.82 (d, J=5.0 Hz, 3H), 2.36-2.23 (m, 2H), 1.94 (s, 3H), 1.40 (br s, 2H), 1.32-1.24 (m, 2H).

### Example 525: (S)-2-Methyl-5-((1-methylazetidin-2-yl)methoxy)-N-(1-(7-vinylquinolin-5-yl)cyclopropyl)benzamide (Compound 570)

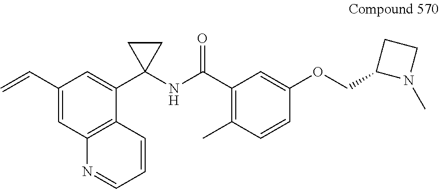 [SMILES: C=Cc1cc(C2(NC(=O)c3cc(OC[C@@H]4CCN4C)ccc3C)CC2)c2cccnc2c1]

Compound 570 was synthesized according to an analogous procedure to the one described for compound 568. M+H⁺=428.2 (LCMS); ¹H NMR (400 MHZ, DMSO-d₆) δ 11.08 (br d, J=2.3 Hz, 1H), 9.62 (br d, J=8.6 Hz, 1H), 9.36 (s, 1H), 9.19 (d, J=4.4 Hz, 1H), 8.29-8.15 (m, 2H), 7.99 (br dd, J=5.1, 8.3 Hz, 1H), 7.17-7.01 (m, 2H), 6.91 (dd, J=2.6, 8.4 Hz, 1H), 6.75 (d, J=2.4 Hz, 1H), 6.23 (d, J=17.5 Hz, 1H), 5.66 (d, J=11.0 Hz, 1H), 4.66-4.59 (m, 1H), 4.46-4.38 (m, 1H), 4.24-4.18 (m, 1H), 4.00-3.92 (m, 1H), 3.88-3.80 (m, 1H), 2.79 (d, J=5.1 Hz, 3H), 2.41-2.23 (m, 2H), 1.97 (s, 3H), 1.40 (br s, 2H), 1.35 (br s, 2H).

### Example 526: (S)-2-Methyl-5-((1-methylazetidin-2-yl)methoxy)-N-(1-(7-(2-methylprop-1-en-1-yl)quinolin-5-yl)cyclopropyl)benzamide (Compound 603)

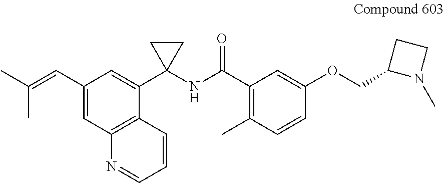 [SMILES: CC(C)=Cc1cc(C2(NC(=O)c3cc(OC[C@@H]4CCN4C)ccc3C)CC2)c2cccnc2c1]

Compound 603 was synthesized according to an analogous procedure to the one described for compound 568. M+H⁺=456.2 (LCMS); ¹H NMR (400 MHZ, DMSO-d₆) δ 9.52 (br d, J=1.3 Hz, 1H), 9.25 (br s, 1H), 9.14 (br s, 1H), 8.06-7.86 (m, 3H), 7.09 (d, J=8.5 Hz, 1H), 6.92 (dd, J=2.7, 8.4 Hz, 1H), 6.74 (d, J=2.3 Hz, 1H), 6.55 (s, 1H), 4.61 (br dd, J=2.8, 6.8 Hz, 1H), 4.36-4.16 (m, 2H), 4.02 (dt, J=4.6, 9.7 Hz, 1H), 3.86 (q, J=9.5 Hz, 1H), 2.90-2.73 (m, 3H), 2.46-2.29 (m, 2H), 2.09-1.90 (m, 9H), 1.41 (br s, 2H), 1.28 (br s, 2H).

### Example 527: (S)-2-Methyl-5-((1-methylazetidin-2-yl)methoxy)-N-(1-(7-(3,3,3-trifluoroprop-1-en-2-yl)quinolin-5-yl)cyclopropyl)benzamide (Compound 650)

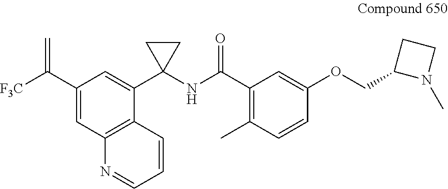 [SMILES: C=C(c1cc(C2(NC(=O)c3cc(OC[C@@H]4CCN4C)ccc3C)CC2)c2cccnc2c1)C(F)(F)F]

Compound 650 was synthesized according to an analogous procedure to the one described for compound 568. M+H⁺=496.2 (LCMS); ¹H NMR (400 MHZ, DMSO-d₆) δ 9.31-9.15 (m, 2H), 9.04 (br d, J=3.8 Hz, 1H), 8.08 (br d, J=5.6 Hz, 2H), 7.74 (br dd, J=4.5, 8.5 Hz, 1H), 7.10 (d, J=8.3 Hz, 1H), 6.92 (dd, J=2.7, 8.3 Hz, 1H), 6.72 (d, J=2.5 Hz, 1H), 6.42 (s, 1H), 6.32 (s, 1H), 4.70-4.55 (m, 1H), 4.35-4.17 (m, 2H), 4.09-3.94 (m, 1H), 3.93-3.81 (m, 1H), 2.69 (s, 3H), 2.34-2.28 (m, 2H), 1.97 (s, 3H), 1.40 (br s, 2H), 1.30 (br s, 2H).

### Example 528: (S)—N-(1-(7-(3-Chlorothiophen-2-yl)quinolin-5-yl)cyclopropyl)-2-methyl-5-((1-methylazetidin-2-yl)methoxy)benzamide (Compound 668)

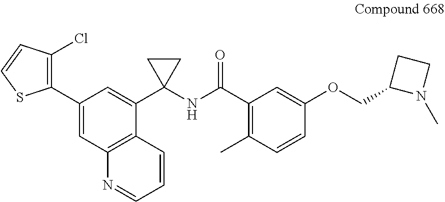 [SMILES: Cc1ccc(OC[C@@H]2CCN2C)cc1C(=O)NC1(c2cc(-c3sccc3Cl)cc3ncccc23)CC1]

Compound 668 was synthesized according to an analogous procedure to the one described for compound 568. M+H⁺=518.2/520.2 (LCMS); ¹H NMR (400 MHZ, DMSO-d₆) δ 10.72-10.46 (m, 1H), 9.44-9.23 (m, 2H), 9.10 (br s, 1H), 8.38 (br s, 1H), 8.26 (s, 1H), 7.90 (d, J=5.4 Hz, 1H), 7.83 (br d, J=3.6 Hz, 1H), 7.31 (d, J=5.4 Hz, 1H), 7.10 (d, J=8.5 Hz, 1H), 6.92 (dd, J=2.7, 8.3 Hz, 1H), 6.74 (d, J=2.7 Hz, 1H), 4.70-4.54 (m, 1H), 4.38-4.30 (m, 1H), 4.22 (br dd, J=3.2, 11.2 Hz, 1H), 4.08-3.97 (m, 2H), 2.81 (d, J=5.0 Hz, 3H), 2.39-2.23 (m, 2H), 1.99 (s, 3H), 1.44 (br s, 2H), 1.31 (br s, 2H).

### Example 529: (S)—N-(1-(7-(4-Chlorothiophen-2-yl)quinolin-5-yl)cyclopropyl)-2-methyl-5-((1-methylazetidin-2-yl)methoxy)benzamide (Compound 632)

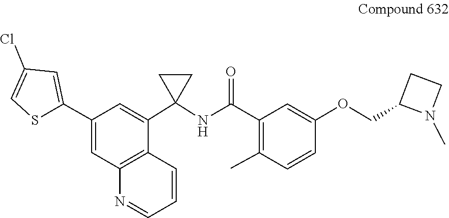 [SMILES: Cc1ccc(OC[C@@H]2CCN2C)cc1C(=O)NC1(c2cc(-c3cc(Cl)cs3)cc3ncccc23)CC1]

Compound 632 was synthesized according to an analogous procedure to the one described for compound 568. M+H⁺=518.2/520.2 (LCMS); ¹H NMR (400 MHZ, DMSO-d₆) δ 10.71-10.56 (m, 1H), 9.39 (br d, J=8.6 Hz, 1H), 9.30 (s, 1H), 9.12 (d, J=3.6 Hz, 1H), 8.32 (s, 1H), 8.28 (d, J=1.6 Hz, 1H), 7.93 (d, J=1.4 Hz, 1H), 7.85 (dd, J=4.6, 8.5 Hz, 1H), 7.80 (d, J=1.4 Hz, 1H), 7.09 (d, J=8.5 Hz, 1H), 6.92 (dd, J=2.8, 8.4 Hz, 1H), 6.74 (d, J=2.8 Hz, 1H), 4.68-4.55 (m, 1H), 4.35 (dd, J=7.9, 11.3 Hz, 1H), 4.21 (dd, J=3.1, 11.3 Hz, 1H), 4.02-3.95 (m, 1H), 3.87-3.83 (m, 1H), 2.81 (d, J=5.0 Hz, 3H), 2.39-2.24 (m, 2H), 1.97 (s, 3H), 1.41 (br d, J=12.6 Hz, 4H).

### Example 530: (S)—N-(1-(7-(5-Chlorothiophen-2-yl)quinolin-5-yl)cyclopropyl)-2-methyl-5-((1-methylazetidin-2-yl)methoxy)benzamide (Compound 623)

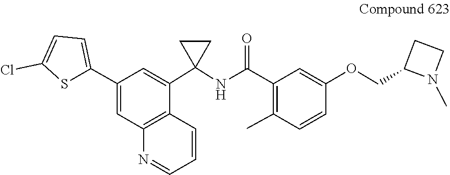 [SMILES: Cc1ccc(OC[C@@H]2CCN2C)cc1C(=O)NC1(c2cc(-c3ccc(Cl)s3)cc3ncccc23)CC1]

Compound 623 was synthesized according to an analogous procedure to the one described for compound 568. M+H⁺=518.2/520.2 (LCMS); ¹H NMR (400 MHZ, DMSO-d₆) δ 9.89-9.77 (m, 1H), 9.17 (s, 1H), 9.04 (br d, J=8.4 Hz, 1H), 8.94 (d, J=4.0 Hz, 1H), 8.10 (d, J=8.9 Hz, 2H), 7.69 (d, J=3.9 Hz, 1H), 7.60 (dd, J=4.6, 8.1 Hz, 1H), 7.27 (d, J=3.9 Hz, 1H), 7.10 (d, J=8.3 Hz, 1H), 6.91 (dd, J=2.6, 8.4 Hz, 1H), 6.70 (d, J=2.6 Hz, 1H), 4.63-4.53 (m, 1H), 4.26-4.15 (m, 2H), 4.07-3.96 (m, 1H), 3.91-3.79 (m, 1H), 2.83 (d, J=4.9 Hz, 3H), 2.39-2.28 (m, 2H), 1.96 (s, 3H), 1.42-1.29 (m, 4H).

### Example 531: N-(1-(7-(Furan-2-yl)quinolin-5-yl)cyclopropyl)-2-methyl-5-((1-methylazetidin-2-yl)methoxy)benzamide (Compound 604)

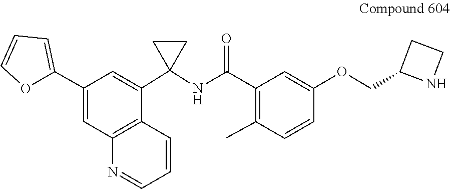 [SMILES: Cc1ccc(OC[C@@H]2CCN2)cc1C(=O)NC1(c2cc(-c3ccco3)cc3ncccc23)CC1]

Compound 604 was synthesized according to an analogous procedure to the one described for compound 568. M+H⁺=4.1 (LCMS); ¹H NMR (400 MHZ, DMSO-d₆) δ 9.50 (br d, J=8.4 Hz, 1H), 9.29 (s, 1H), 9.20-9.12 (m, 1H), 8.37 (d, J=1.4 Hz, 1H), 8.32 (s, 1H), 7.97 (d, J=1.5 Hz, 1H), 7.92 (dd, J=4.9, 8.6 Hz, 1H), 7.42 (d, J=3.4 Hz, 1H), 7.12-7.07 (m, 1H), 6.98-6.88 (m, 1H), 6.79-6.75 (m, 1H), 6.74 (d, J=2.6 Hz, 1H), 4.70-4.56 (m, 1H), 4.33-4.17 (m, 2H), 4.01 (dt, J=4.6, 9.6 Hz, 1H), 3.86 (q, J=9.5 Hz, 1H), 2.84-2.68 (m, 3H), 2.42-2.22 (m, 2H), 1.95 (s, 3H), 1.48-1.33 (m, 4H).

### Example 532: (S)—N-(1-(7-(1H-Pyrazol-4-yl)quinolin-5-yl)cyclopropyl)-2-methyl-5-((1-methylazetidin-2-yl)methoxy)benzamide (Compound 652)

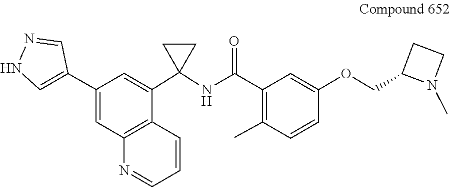 [SMILES: Cc1ccc(OC[C@@H]2CCN2C)cc1C(=O)NC1(c2cc(-c3cn[nH]c3)cc3ncccc23)CC1]

Compound 652 was synthesized according to an analogous procedure to the one described for compound 568. M+H⁺=468.2 (LCMS); ¹H NMR (400 MHZ, DMSO-d₆) δ 9.21-9.04 (m, 2H), 8.94 (d, J=4.1 Hz, 1H), 8.31 (br s, 2H), 8.19-8.06 (m, 2H), 7.67-7.49 (m, 1H), 7.10 (d, J=8.5 Hz, 1H), 6.91 (dd, J=2.7, 8.3 Hz, 1H), 6.70 (d, J=2.6 Hz, 1H), 4.69-4.53 (m, 1H), 4.22 (br d, J=3.0 Hz, 2H), 4.06-3.99 (m, 1H), 3.94-3.85 (m, 1H), 2.88-2.79 (m, 3H), 2.48-2.43 (m, 1H), 2.35 (br s, 1H), 1.98 (s, 3H), 1.50-1.25 (m, 4H).

### Example 533: (S)-2-Methyl-N-(1-(7-(1-methyl-1H-pyrazol-4-yl)quinolin-5-yl)cyclopropyl)-5-((1-methylazetidin-2-yl)methoxy)benzamide (Compound 670)

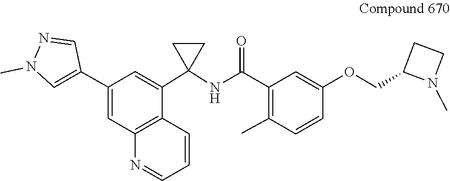

Compound 670 was synthesized according to an analogous procedure to the one described for compound 568. M+H⁺=482.3 (LCMS); ¹H NMR (400 MHZ, DMSO-d₆) δ 10.45-10.28 (m, 1H), 9.46-9.33 (m, 1H), 9.25 (s, 1H), 9.08 (br d, J=2.1 Hz, 1H), 8.50 (s, 1H), 8.20 (br d, J=18.0 Hz, 2H), 8.13 (s, 1H), 7.86-7.77 (m, 1H), 7.14-7.07 (m, 1H), 6.92 (dd, J=2.7, 8.3 Hz, 1H), 6.74 (d, J=2.5 Hz, 1H), 4.66-4.57 (m, 1H), 4.36-4.19 (m, 2H), 4.06-3.98 (m, 1H), 3.95 (s, 3H), 3.86 (br dd, J=6.5, 9.6 Hz, 1H), 2.82 (d, J=4.9 Hz, 3H), 2.34-2.26 (m, 2H), 1.98 (s, 3H), 1.40 (br d, J=16.6 Hz, 4H).

### Example 534: (S)—N-(1-(7-(1H-Pyrazol-3-yl)quinolin-5-yl)cyclopropyl)-2-methyl-5-((1-methylazetidin-2-yl)methoxy)benzamide (Compound 633)

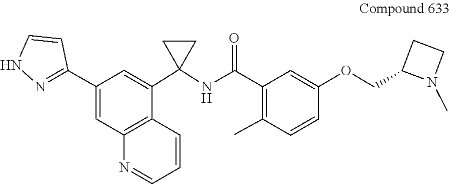 [SMILES: Cc1ccc(OC[C@@H]2CCN2C)cc1C(=O)NC1(c2cc(-c3cc[nH]n3)cc3ncccc23)CC1]

Compound 633 was synthesized according to an analogous procedure to the one described for compound 568. M+H⁺=468.3 (LCMS); ¹H NMR (400 MHZ, DMSO-d₆) δ 10.59-10.39 (m, 1H), 9.45 (br d, J=5.5 Hz, 1H), 9.28 (s, 1H), 9.17-9.02 (m, 1H), 8.49 (br d, J=17.9 Hz, 2H), 7.95-7.77 (m, 2H), 7.17-7.06 (m, 1H), 6.99 (d, J=2.1 Hz, 1H), 6.92 (dd, J=2.8, 8.4 Hz, 1H), 6.81-6.67 (m, 1H), 4.72-4.55 (m, 1H), 4.39-4.27 (m, 1H), 4.26-4.15 (m, 1H), 4.03-3.96 (m, 1H), 3.85 (br dd, J=6.7, 9.7 Hz, 1H), 2.81 (d, J=4.9 Hz, 3H), 2.42-2.34 (m, 1H), 2.32-2.22 (m, 1H), 1.97 (s, 3H), 1.44 (br s, 2H), 1.35 (br s, 2H).

### Example 535: (S)-2-Methyl-N-(1-(7-(5-methyl-1H-pyrazol-3-yl)quinolin-5-yl)cyclopropyl)-5-((1-methylazetidin-2-yl)methoxy)benzamide (Compound 651)

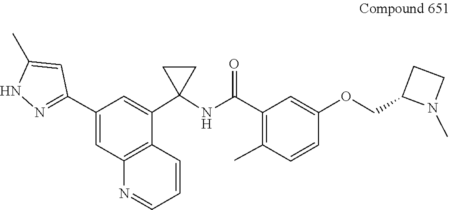

Compound 651 was synthesized according to an analogous procedure to the one described for compound 568. M+H⁺=482.3 (LCMS); ¹H NMR (400 MHZ, DMSO-d₆) δ 12.73 (s, 1H), 9.11 (s, 1H), 9.04 (br d, J=8.4 Hz, 1H), 8.94-8.85 (m, 1H), 8.35 (s, 1H), 8.21 (s, 1H), 7.52 (dd, J=4.3, 8.5 Hz, 1H), 7.02 (d, J=8.5 Hz, 1H), 6.83 (dd, J=2.8, 8.3 Hz, 1H), 6.66 (s, 1H), 6.61 (d, J=2.6 Hz, 1H), 3.86 (d, J=5.4 Hz, 2H), 3.27-3.16 (m, 2H), 2.76-2.66 (m, 1H), 2.32 (s, 3H), 2.23-2.17 (m, 3H), 1.98-1.92 (m, 4H), 1.88-1.80 (m, 1H), 1.39 (br s, 2H), 1.33-1.20 (m, 2H).

### Example 536: (S)-2-Methyl-N-(1-(7-(1-methyl-1H-pyrazol-3-yl)quinolin-5-yl)cyclopropyl)-5-((1-methylazetidin-2-yl)methoxy)benzamide (Compound 582)

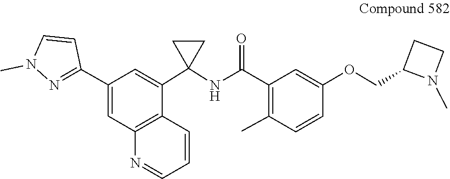

Compound 582 was synthesized according to an analogous procedure to the one described for compound 568. M+H⁺=482.1 (LCMS); ¹H NMR (400 MHZ, DMSO-d₆) δ 9.64 (br d, J=8.8 Hz, 1H), 9.36-9.29 (m, 1H), 9.20 (d, J=4.9 Hz, 1H), 8.56 (d, J=1.4 Hz, 1H), 8.48 (s, 1H), 7.99 (dd, J=5.1, 8.4 Hz, 1H), 7.90 (d, J=2.3 Hz, 1H), 7.09 (d, J=8.5 Hz, 1H), 7.00 (d, J=2.3 Hz, 1H), 6.91 (dd, J=2.6, 8.4 Hz, 1H), 6.75 (d, J=2.6 Hz, 1H), 4.62 (br dd, J=2.9, 7.8 Hz, 1H), 4.39-4.28 (m, 1H), 4.25-4.18 (m, 1H), 4.04-3.95 (m, 4H), 3.85 (d, J=9.8 Hz, 1H), 2.84-2.68 (m, 3H), 2.36 (br s, 2H), 1.96 (s, 3H), 1.46 (br s, 2H), 1.35 (br s, 2H).

### Example 537: (S)—N-(1-(7-(1-(Difluoromethyl)-1H-pyrazol-3-yl)quinolin-5-yl)cyclopropyl)-2-methyl-5-((1-methylazetidin-2-yl)methoxy)benzamide (Compound 644)

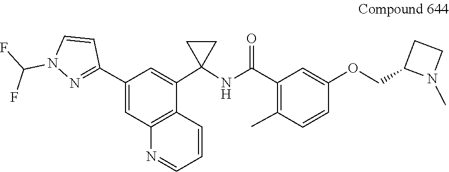 [SMILES: Cc1ccc(OC[C@@H]2CCN2C)cc1C(=O)NC1(c2cc(-c3ccn(C(F)F)n3)cc3ncccc23)CC1]

Compound 644 was synthesized according to an analogous procedure to the one described for compound 568. M+H⁺=518.2 (LCMS); ¹H NMR (400 MHZ, DMSO-d₆) δ 9.47 (br d, J=8.4 Hz, 1H), 9.29 (s, 1H), 9.18-9.10 (m, 1H), 8.55 (s, 2H), 8.44 (d, J=2.8 Hz, 1H), 8.15-7.78 (m, 2H), 7.32 (d, J=2.6 Hz, 1H), 7.09 (d, J=8.5 Hz, 1H), 6.92 (dd, J=2.8, 8.4 Hz, 1H), 6.74 (d, J=2.8 Hz, 1H), 4.62 (br dd, J=3.3, 7.7 Hz, 1H), 4.37-4.17 (m, 2H), 4.00 (td, J=4.9, 9.5 Hz, 1H), 3.94-3.79 (m, 1H), 2.88-2.67 (m, 3H), 2.39-2.28 (m, 2H), 1.96 (s, 3H), 1.45 (br s, 2H), 1.34 (br s, 2H).

### Example 538: (S)-2-Methyl-5-((1-methylazetidin-2-yl)methoxy)-N-(1-(7-(1-phenyl-1H-pyrazol-3-yl)quinolin-5-yl)cyclopropyl)benzamide (Compound 659)

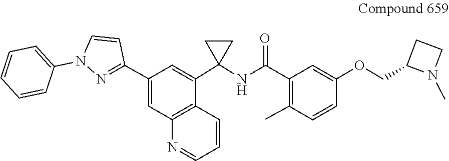 [SMILES: Cc1ccc(OC[C@@H]2CCN2C)cc1C(=O)NC1(c2cc(-c3ccn(-c4ccccc4)n3)cc3ncccc23)CC1]

Compound 659 was synthesized according to an analogous procedure to the one described for compound 568. M+H⁺=544.3 (LCMS); ¹H NMR (400 MHZ, CD₃OD) δ 9.15 (d, J=8.5 Hz, 1H), 8.89 (dd, J=1.5, 4.3 Hz, 1H), 8.65 (d, J=1.6 Hz, 1H), 8.48 (s, 1H), 8.36 (d, J=2.5 Hz, 1H), 7.93 (d, J=7.8 Hz, 2H), 7.60 (dd, J=4.3, 8.5 Hz, 1H), 7.54 (t, J=8.0 Hz, 2H), 7.40-7.33 (m, 1H), 7.15 (d, J=2.5 Hz, 1H), 7.07 (d, J=8.5 Hz, 1H), 6.88 (dd, J=2.8, 8.4 Hz, 1H), 6.70 (d, J=2.6 Hz, 1H), 4.06-3.97 (m, 2H), 3.85 (br d, J=1.1 Hz, 1H), 3.62 (td, J=3.9, 7.9 Hz, 1H), 3.28-3.21 (m, 1H), 2.54 (s, 3H), 2.25-2.14 (m, 2H), 2.02 (s, 3H), 1.57-1.52 (m, 2H), 1.48-1.40 (m, 2H).

### Example 539: (S)—N-(1-(7-(Cyclopent-1-en-1-yl)quinolin-5-yl)cyclopropyl)-2-methyl-5-((1-methylazetidin-2-yl)methoxy)benzamide (Compound 609)

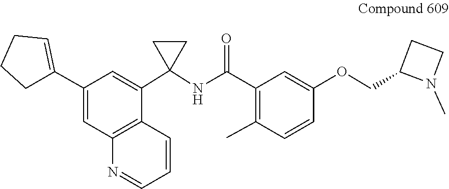 [SMILES: Cc1ccc(OC[C@@H]2CCN2C)cc1C(=O)NC1(c2cc(C3=CCCC3)cc3ncccc23)CC1]

Compound 609 was synthesized according to an analogous procedure to the one described for compound 568. M+H⁺=468.1 (LCMS); ¹H NMR (400 MHZ, DMSO-d₆) δ 9.29-9.16 (m, 2H), 9.06-8.97 (m, 1H), 8.22-8.16 (m, 1H), 7.90 (br d, J=5.8 Hz, 1H), 7.65 (br d, J=1.3 Hz, 1H), 7.13-7.05 (m, 1H), 6.97-6.87 (m, 1H), 6.72-6.69 (m, 1H), 6.67-6.64 (m, 1H), 4.65-4.50 (m, 1H), 4.33-4.17 (m, 2H), 4.06-3.94 (m, 1H), 3.90-3.75 (m, 2H), 2.85-2.75 (m, 5H), 2.62-2.57 (m, 2H), 2.37-2.25 (m, 2H), 2.12-2.01 (m, 2H), 1.95 (s, 3H), 1.38 (br s, 2H), 1.31-1.23 (m, 2H).

### Example 540: (S)—N-(1-(7-(Benzo[b]thiophen-2-yl)quinolin-5-yl)cyclopropyl)-2-methyl-5-((1-methylazetidin-2-yl)methoxy)benzamide (Compound 621)

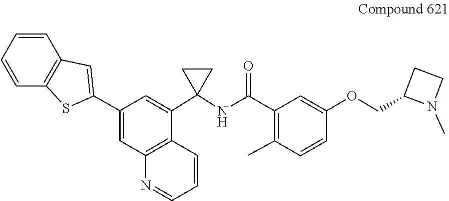 [SMILES: Cc1ccc(OC[C@@H]2CCN2C)cc1C(=O)NC1(c2cc(-c3cc4ccccc4s3)cc3ncccc23)CC1]

Compound 621 was synthesized according to an analogous procedure to the one described for compound 568. M+H⁺=534.2 (LCMS); ¹H NMR (400 MHZ, CD₃OD) δ 9.90 (br d, J=8.3 Hz, 1H), 9.43 (br s, 1H), 9.22 (br d, J=5.5 Hz, 1H), 8.80 (s, 1H), 8.39 (s, 1H), 8.25 (s, 1H), 8.18-8.08 (m, 1H), 7.97 (dd, J=3.2, 6.0 Hz, 2H), 7.46 (dd, J=3.1, 6.1 Hz, 2H), 7.14 (d, J=8.4 Hz, 1H), 6.98 (dd, J=2.7, 8.3 Hz, 1H), 6.88 (s, 1H), 4.74-4.65 (m, 1H), 4.38-4.14 (m, 3H), 4.02-3.88 (m, 1H), 2.95 (s, 3H), 2.62-2.48 (m, 2H), 2.07 (s, 3H), 1.66 (br s, 2H), 1.59-1.52 (m, 2H).

### Example 541: (S)-2-Methyl-5-((1-methylazetidin-2-yl)methoxy)-N-(1-(7-phenylquinolin-5-yl)cyclopropyl)benzamide (Compound 594)

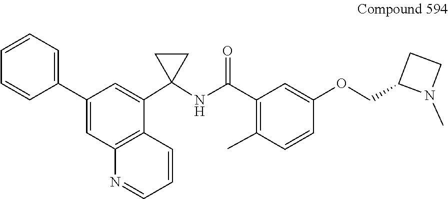 [SMILES: Cc1ccc(OC[C@@H]2CCN2C)cc1C(=O)NC1(c2cc(-c3ccccc3)cc3ncccc23)CC1]

Compound 594 was synthesized according to an analogous procedure to the one described for compound 568. M+H⁺=478.1 (LCMS); ¹H NMR (400 MHZ, DMSO-d₆) δ 9.50 (br d, J=8.0 Hz, 1H), 9.29 (s, 1H), 9.19-9.11 (m, 1H), 8.39-8.27 (m, 2H), 7.97-7.84 (m, 3H), 7.66-7.58 (m, 2H), 7.55-7.49 (m, 1H), 7.18-7.05 (m, 1H), 6.92 (dd, J=2.8, 8.4 Hz, 1H), 6.81-6.71 (m, 1H), 4.68-4.56 (m, 1H), 4.34-4.17 (m, 2H), 4.01 (dt, J=4.6, 9.6 Hz, 1H), 3.85 (q, J=9.3 Hz, 1H), 2.84-2.69 (m, 3H), 2.40-2.28 (m, 2H), 1.97 (s, 3H), 1.48-1.36 (m, 4H).

### Example 542: (R)-5-(2-(Dimethylamino)propoxy)-N-(1-(7-methoxyquinolin-5-yl)cyclopropyl)-2-methylbenzamide (Compound 574)

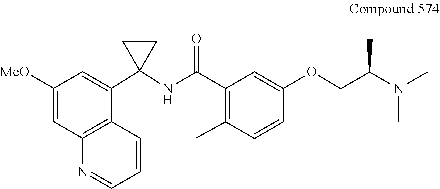 [SMILES: COc1cc(C2(NC(=O)c3cc(OC[C@@H](C)N(C)C)ccc3C)CC2)c2cccnc2c1]

Compound 574 was synthesized according to an analogous procedure to the one described for compound 575. M+H⁺=434.1 (LCMS); ¹H NMR (400 MHZ, DMSO-d₆) δ 10.87-10.59 (m, 1H), 9.77 (d, J=8.51 Hz, 1H), 9.42 (m, 1H), 9.20 (m, 1H), 8.00 (m, 1H), 7.84-7.61 (m, 2H), 7.09 (d, J=8.50 Hz, 1H), 6.93 (m, 1H), 6.80 (d, J=2.63 Hz, 1H), 4.20 (m, 2H), 4.02 (m, 3H), 3.74-3.68 (m, 1H), 2.76-2.69 (m, 6H), 1.97 (m, 3H), 1.43 (m, 2H), 1.31 (d, J=6.75 Hz, 5H).

### Example 543: (S)-2-Methyl-5-((1-methylazetidin-2-yl)methoxy)-N-(1-(7-phenoxyquinolin-5-yl)cyclopropyl)benzamide (Compound 592)

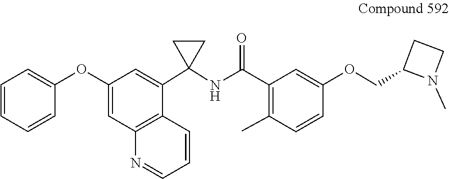 [SMILES: Cc1ccc(OC[C@@H]2CCN2C)cc1C(=O)NC1(c2cc(Oc3ccccc3)cc3ncccc23)CC1]

Compound 592 was synthesized according to an analogous procedure to the one described for compound 581. M+H⁺=494.2. 1H NMR (400 MHZ, DMSO-d₆) δ 9.15 (s, 1H), 9.07-8.95 (m, 1H), 8.88-8.75 (m, 1H), 7.76-7.61 (m, 1H), 7.55-7.43 (m, 3H), 7.35-7.13 (m, 4H), 7.09-6.98 (m, 1H), 6.91-6.77 (m, 1H), 6.69-6.56 (m, 1H), 3.95-3.78 (m, 2H), 3.26-3.19 (m, 2H), 2.76-2.69 (m, 1H), 2.24-2.18 (m, 3H), 2.03-1.90 (m, 4H), 1.88-1.79 (m, 1H), 1.42-1.29 (m, 2H), 1.25-1.20 (m, 2H).

### Example 544: (S)-2-Methyl-5-((1-methylazetidin-2-yl)methoxy)-N-(1-(7-(thiazol-2-yl)quinolin-5-yl)cyclopropyl)benzamide (Compound 590)

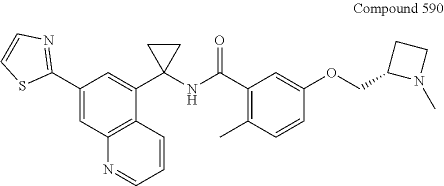 [SMILES: Cc1ccc(OC[C@@H]2CCN2C)cc1C(=O)NC1(c2cc(-c3nccs3)cc3ncccc23)CC1]

Compound 590 was synthesized according to an analogous procedure to the one described for compound 588. M+H⁺=485.1 (LCMS); ¹H NMR (400 MHZ, DMSO-d₆) δ 10.59-10.41 (m, 1H), 9.38-9.24 (m, 2H), 9.09 (d, J=3.3 Hz, 1H), 8.58-8.46 (m, 2H), 8.08 (d, J=3.1 Hz, 1H), 7.97 (d, J=3.3 Hz, 1H), 7.80 (dd, J=4.3, 8.5 Hz, 1H), 7.09 (d, J=8.5 Hz, 1H), 6.92 (dd, J=2.8, 8.4 Hz, 1H), 6.74 (d, J=2.6 Hz, 1H), 4.69-4.54 (m, 1H), 4.39-4.19 (m, 2H), 4.04-3.93 (m, 1H), 3.88-3.81 (m, 1H), 2.81 (d, J=4.9 Hz, 3H), 2.40-2.25 (m, 2H), 1.97 (s, 3H), 1.45 (br s, 2H), 1.32 (br s, 2H).

### Example 545: (S)—N-(1-(7-(2-Methoxyethoxy)quinolin-5-yl)cyclopropyl)-2-methyl-5-((1-methylazetidin-2-yl)methoxy)benzamide (Compound 591)

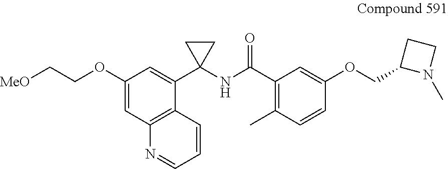 [SMILES: COCCOc1cc(C2(NC(=O)c3cc(OC[C@@H]4CCN4C)ccc3C)CC2)c2cccnc2c1]

Compound 591 was synthesized according to an analogous procedure to the one described for compound 598. M+H⁺=476.1 (LCMS); ¹H NMR (400 MHZ, DMSO-d₆) δ 10.48-10.28 (m, 1H), 9.39 (br dd, J=2.5, 4.8 Hz, 1H), 9.30-9.20 (m, 1H), 9.05 (br d, J=4.3 Hz, 1H), 7.85-7.72 (m, 1H), 7.62 (s, 1H), 7.50-7.43 (m, 1H), 7.14-7.06 (m, 1H), 6.98-6.89 (m, 1H), 6.72 (s, 1H), 4.67-4.55 (m, 1H), 4.36-4.27 (m, 3H), 4.26-4.19 (m, 1H), 4.03-3.96 (m, 1H), 3.90-3.84 (m, 1H), 3.79-3.76 (m, 2H), 2.82 (d, J=5.0 Hz, 3H), 2.72-2.63 (m, 2H), 2.42-2.24 (m, 3H), 1.95 (s, 3H), 1.42-1.36 (m, 2H), 1.28 (br s, 2H).

### Example 546: (S)-5-(Azetidin-2-ylmethoxy)-2-methyl-N-(1-(7-vinylquinolin-5-yl)cyclopropyl)benzamide (Compound 612)

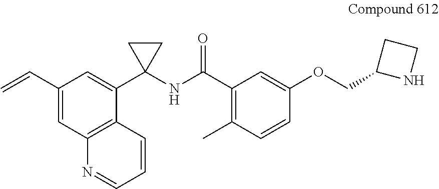 [SMILES: C=Cc1cc(C2(NC(=O)c3cc(OC[C@@H]4CCN4)ccc3C)CC2)c2cccnc2c1]

Compound 612 was synthesized according to an analogous procedure to the one described for compound 608. M+H⁺=414.2 (LCMS); ¹H NMR (400 MHZ, DMSO-d₆) δ 9.27 (d, J=8.5 Hz, 1H), 9.19 (s, 1H), 9.04 (dd, J=1.2, 4.4 Hz, 1H), 8.14 (d, J=1.4 Hz, 1H), 8.00 (s, 1H), 7.75 (dd, J=4.6, 8.6 Hz, 1H), 7.12-6.98 (m, 2H), 6.91 (dd, J=2.8, 8.4 Hz, 1H), 6.70 (d, J=2.8 Hz, 1H), 6.15 (d, J=17.6 Hz, 1H), 5.56 (d, J=11.0 Hz, 1H), 4.70-4.60 (m, 1H), 4.28-4.19 (m, 1H), 4.17-4.09 (m, 1H), 3.99-3.79 (m, 2H), 2.44-2.30 (m, 2H), 1.96 (s, 3H), 1.39 (br s, 2H), 1.31 (br s, 2H).

### Example 547: (S)-5-(Azetidin-2-ylmethoxy)-2-methyl-N-(1-(7-(prop-1-en-2-yl)quinolin-5-yl)cyclopropyl)benzamide (Compound 613)

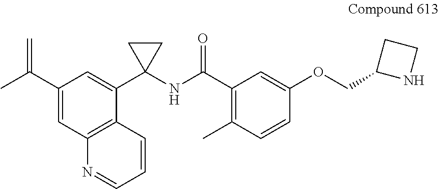 [SMILES: C=C(C)c1cc(C2(NC(=O)c3cc(OC[C@@H]4CCN4)ccc3C)CC2)c2cccnc2c1]

Compound 613 was synthesized according to an analogous procedure to the one described for compound 608. M+H⁺=428.2 (LCMS); ¹H NMR (400 MHZ, DMSO-d₆) δ 9.32 (d, J=8.5 Hz, 1H), 9.19 (s, 1H), 9.07 (dd, J=1.4, 4.6 Hz, 1H), 8.20 (d, J=1.6 Hz, 1H), 8.04 (s, 1H), 7.79 (dd, J=4.6, 8.6 Hz, 1H), 7.10 (d, J=8.4 Hz, 1H), 6.91 (dd, J=2.8, 8.4 Hz, 1H), 6.70 (d, J=2.8 Hz, 1H), 5.79 (s, 1H), 5.42 (s, 1H), 4.65 (dq, J=3.4, 7.8 Hz, 1H), 4.27-4.19 (m, 1H), 4.17-4.10 (m, 1H), 3.96-3.88 (m, 1H), 3.82 (dt, J=6.3, 10.0 Hz, 1H), 2.45-2.38 (m, 1H), 2.37-2.31 (m, 1H), 2.27 (s, 3H), 1.97 (s, 3H), 1.40 (br s, 2H), 1.31 (br s, 2H).

### Example 548: (S,E)-5-(Azetidin-2-ylmethoxy)-N-(1-(7-(2-cyclopropylvinyl)quinolin-5-yl)cyclopropyl)-2-methylbenzamide (Compound 667)

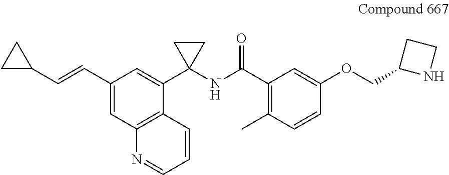 [SMILES: Cc1ccc(OC[C@@H]2CCN2)cc1C(=O)NC1(c2cc(/C=C/C3CC3)cc3ncccc23)CC1]

Compound 667 was synthesized according to an analogous procedure to the one described for compound 608. M+H⁺=454.3 (LCMS); ¹H NMR (400 MHZ, DMSO-d₆) 9.23 (br d, J=6.1 Hz, 2H), 9.00-8.74 (m, 3H), 7.96 (br d, J=3.9 Hz, 1H), 7.80 (s, 1H), 7.69-7.57 (m, 1H), 7.10 (d, J=8.5 Hz, 1H), 6.91 (dd, J=2.8, 8.4 Hz, 1H), 6.78-6.66 (m, 2H), 6.24-6.09 (m, 1H), 4.73-4.60 (m, 1H), 4.26-4.09 (m, 2H), 3.92 (br d, J=6.1 Hz, 1H), 3.85-3.81 (m, 1H), 2.46-2.30 (m, 2H), 1.97 (s, 3H), 1.76-1.64 (m, 1H), 1.41-1.23 (m, 4H), 0.92-0.85 (m, 2H), 0.70-0.60 (m, 2H).

### Example 549: (S,E)-5-(Azetidin-2-ylmethoxy)-2-methyl-N-(1-(7-(3-methylstyryl)quinolin-5-yl)cyclopropyl)benzamide (Compound 704)

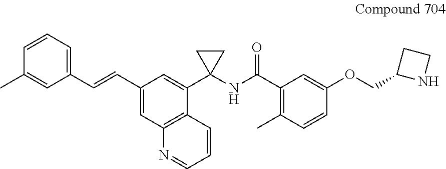 [SMILES: Cc1cccc(/C=C/c2cc(C3(NC(=O)c4cc(OC[C@@H]5CCN5)ccc4C)CC3)c3cccnc3c2)c1]

Compound 704 was synthesized according to an analogous procedure to the one described for compound 608. M+H⁺=504.4 (LCMS); ¹H NMR (400 MHZ, DMSO-d₆) δ 9.48 (d, J=8.3 Hz, 1H), 9.02 (dd, J=1.3, 4.8 Hz, 1H), 8.51 (d, J=1.3 Hz, 1H), 8.09 (s, 1H), 7.83 (dd, J=4.9, 8.5 Hz, 1H), 7.65-7.57 (m, 1H), 7.55-7.42 (m, 3H), 7.31 (t, J=7.7 Hz, 1H), 7.18 (br d, J=7.5 Hz, 1H), 7.13 (d, J=8.4 Hz, 1H), 6.96 (dd, J=2.7, 8.5 Hz, 1H), 6.80 (d, J=2.8 Hz, 1H), 4.80-4.75 (m, 1H), 4.26-4.19 (m, 2H), 4.09-3.94 (m, 2H), 2.65-2.52 (m, 2H), 2.41 (s, 3H), 2.04 (s, 3H), 1.60-1.51 (m, 2H), 1.50-1.43 (m, 2H).

### Example 550: (S)-5-(Azetidin-2-ylmethoxy)-2-methyl-N-(1-(7-(5-methylthiophen-3-yl)quinolin-5-yl)cyclopropyl)benzamide (Compound 640)

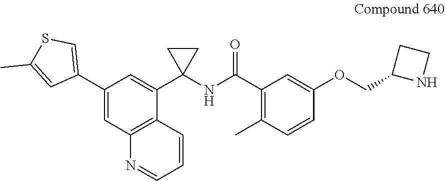 [SMILES: Cc1cc(-c2cc(C3(NC(=O)c4cc(OC[C@@H]5CCN5)ccc4C)CC3)c3cccnc3c2)cs1]

Compound 640 was synthesized according to an analogous procedure to the one described for compound 608. M+H⁺=484.1 (LCMS); ¹H NMR (400 MHZ, DMSO-d₆) δ 9.21-9.10 (m, 2H), 8.97 (dd, J=1.3, 4.3 Hz, 1H), 8.95-8.69 (m, 2H), 8.26-8.17 (m, 2H), 7.91 (d, J=1.5 Hz, 1H), 7.64 (dd, J=4.3, 8.6 Hz, 1H), 7.47 (s, 1H), 7.10 (d, J=8.5 Hz, 1H), 6.91 (dd, J=2.8, 8.4 Hz, 1H), 6.70 (d, J=2.8 Hz, 1H), 4.70-4.61 (m, 1H), 4.27-4.19 (m, 1H), 4.17-4.10 (m, 1H), 3.98-3.87 (m, 2H), 2.55 (d, J=0.8 Hz, 3H), 2.44-2.29 (m, 2H), 1.98 (s, 3H), 1.39 (br s, 2H), 1.37-1.31 (m, 2H).

### Example 551: (S)—N-(1-(7-(5-Acetylthiophen-2-yl)quinolin-5-yl)cyclopropyl)-5-(azetidin-2-ylmethoxy)-2-methylbenzamide (Compound 643)

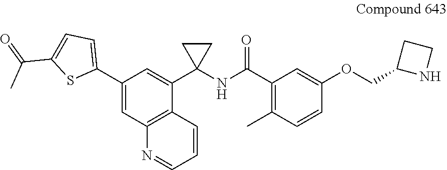 [SMILES: CC(=O)c1ccc(-c2cc(C3(NC(=O)c4cc(OC[C@@H]5CCN5)ccc4C)CC3)c3cccnc3c2)s1]

Compound 643 was synthesized according to an analogous procedure to the one described for compound 608. M+H⁺=512.2 (LCMS); ¹H NMR (400 MHZ, DMSO-d₆) δ 9.19 (s, 1H), 9.08 (d, J=8.7 Hz, 1H), 8.98 (dd, J=1.6, 4.1 Hz, 1H), 8.82-8.75 (m, 1H), 8.32 (d, J=1.4 Hz, 1H), 8.21 (d, J=1.9 Hz, 1H), 8.05 (d, J=4.0 Hz, 1H), 7.93 (d, J=4.0 Hz, 1H), 7.65 (dd, J=4.3, 8.5 Hz, 1H), 7.10 (d, J=8.5 Hz, 1H), 6.91 (dd, J=2.7, 8.3 Hz, 1H), 6.70 (d, J=2.8 Hz, 1H), 4.69-4.60 (m, 1H), 4.26-4.19 (m, 1H), 4.17-4.10 (m, 1H), 3.96-3.88 (m, 1H), 3.86-3.79 (m, 1H), 2.69-2.65 (m, 1H), 2.59 (s, 3H), 2.34-2.31 (m, 1H), 1.98 (s, 3H), 1.43-1.38 (m, 2H), 1.35 (br s, 2H).

### Example 552: (S)-5-(Azetidin-2-ylmethoxy)-N-(1-(7-(5-cyanothiophen-2-yl)quinolin-5-yl)cyclopropyl)-2-methylbenzamide (Compound 658)

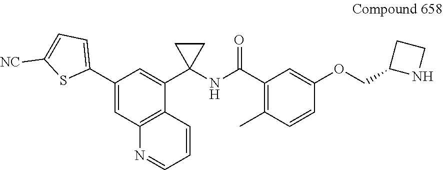 [SMILES: Cc1ccc(OC[C@@H]2CCN2)cc1C(=O)NC1(c2cc(-c3ccc(C#N)s3)cc3ncccc23)CC1]

Compound 658 was synthesized according to an analogous procedure to the one described for compound 608. M+H⁺=495.3 (LCMS); ¹H NMR (400 MHZ, DMSO-d₆) δ 9.20 (s, 1H), 9.08 (d, J=8.6 Hz, 1H), 8.99 (dd, J=1.4, 4.2 Hz, 1H), 8.89-8.71 (m, 2H), 8.33 (d, J=1.5 Hz, 1H), 8.17 (d, J=1.9 Hz, 1H), 8.10 (d, J=4.0 Hz, 1H), 7.98 (d, J=4.0 Hz, 1H), 7.66 (dd, J=4.2, 8.6 Hz, 1H), 7.13-7.06 (m, 1H), 6.91 (dd, J=2.7, 8.3 Hz, 1H), 6.70 (d, J=2.6 Hz, 1H), 4.70-4.57 (m, 1H), 4.28-4.18 (m, 1H), 4.17-4.09 (m, 1H), 3.98-3.78 (m, 2H), 2.48-2.42 (m, 1H), 2.48-2.40 (m, 2H), 2.24 (s, 1H), 1.97 (s, 3H), 1.40 (br s, 2H), 1.35 (br s, 2H).

### Example 553: (S)-5-(Azetidin-2-ylmethoxy)-2-methyl-N-(1-(7-(5-methyl-1,3,4-thiadiazol-2-yl)quinolin-5-yl)cyclopropyl)benzamide (Compound 673)

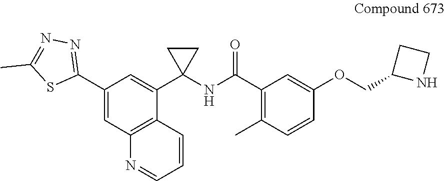 [SMILES: Cc1nnc(-c2cc(C3(NC(=O)c4cc(OC[C@@H]5CCN5)ccc4C)CC3)c3cccnc3c2)s1]

Compound 673 was synthesized according to an analogous procedure to the one described for compound 608. M+H⁺=486.1 (LCMS); ¹H NMR (400 MHZ, DMSO-d₆) δ 9.23 (s, 1H), 9.16 (d, J=8.5 Hz, 1H), 9.03 (dd, J=1.4, 4.1 Hz, 1H), 9.00-8.73 (m, 2H), 8.48 (d, J=1.8 Hz, 1H), 8.42 (d, J=1.1 Hz, 1H), 7.72 (dd, J=4.2, 8.6 Hz, 1H), 7.09 (d, J=8.5 Hz, 1H), 6.91 (dd, J=2.7, 8.3 Hz, 1H), 6.72 (d, J=2.6 Hz, 1H), 4.65 (br d, J=5.1 Hz, 1H), 4.28-4.19 (m, 1H), 4.18-4.09 (m, 1H), 3.96-3.89 (m, 1H), 3.86-3.80 (m, 1H), 2.84 (s, 3H), 2.48-2.28 (m, 2H), 1.96 (s, 3H), 1.43 (br s, 2H), 1.31 (br s, 2H).

### Example 554: (S)-5-(Azetidin-2-ylmethoxy)-N-(1-(7-(furan-3-yl)quinolin-5-yl)cyclopropyl)-2-methylbenzamide (Compound 645)

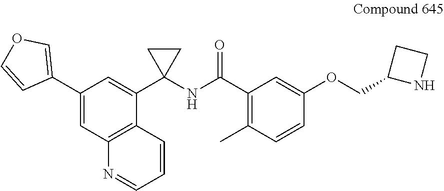 [SMILES: Cc1ccc(OC[C@@H]2CCN2)cc1C(=O)NC1(c2cc(-c3ccoc3)cc3ncccc23)CC1]

Compound 645 was synthesized according to an analogous procedure to the one described for compound 608. M+H⁺=454.2 (LCMS); ¹H NMR (400 MHZ, DMSO-d₆) δ 9.25-9.09 (m, 2H), 9.03-8.71 (m, 3H), 8.48 (s, 1H), 8.27-8.06 (m, 2H), 7.86 (t, J=1.6 Hz, 1H), 7.65 (dd, J=4.3, 8.5 Hz, 1H), 7.18 (d, J=1.1 Hz, 1H), 7.10 (d, J=8.5 Hz, 1H), 6.91 (dd, J=2.8, 8.4 Hz, 1H), 6.70 (d, J=2.6 Hz, 1H), 4.65 (br d, J=4.0 Hz, 1H), 4.23 (br dd, J=7.1, 11.2 Hz, 2H), 4.13 (br dd, J=3.3, 11.2 Hz, 2H), 2.45-2.24 (m, 2H), 1.98 (s, 3H), 1.39 (br s, 2H), 1.35 (br s, 2H).

### Example 555: (S)-5-(Azetidin-2-ylmethoxy)-N-(1-(7-(2,5-dimethylfuran-3-yl)quinolin-5-yl)cyclopropyl)-2-methylbenzamide (Compound 641)

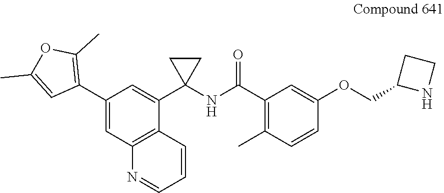 [SMILES: Cc1cc(-c2cc(C3(NC(=O)c4cc(OC[C@@H]5CCN5)ccc4C)CC3)c3cccnc3c2)c(C)o1]

Compound 641 was synthesized according to an analogous procedure to the one described for compound 608. M+H⁺=482.1 (LCMS); ¹H NMR (400 MHZ, DMSO-d₆) δ 9.21-9.09 (m, 2H), 8.96 (br d, J=4.0 Hz, 1H), 8.87-8.71 (m, 2H), 8.03 (s, 1H), 7.93 (s, 1H), 7.63 (br dd, J=3.9, 8.0 Hz, 1H), 7.10 (d, J=8.5 Hz, 1H), 6.91 (dd, J=2.7, 8.3 Hz, 1H), 6.71 (d, J=2.8 Hz, 1H), 6.51 (s, 1H), 4.71-4.60 (m, 1H), 4.27-4.19 (m, 1H), 4.16-4.10 (m, 1H), 3.96-3.87 (m, 1H), 3.86-3.77 (m, 1H), 2.54 (s, 3H), 2.39-2.32 (m, 2H), 2.31 (s, 3H), 1.99 (s, 3H), 1.39 (br s, 2H), 1.32-1.25 (m, 2H).

### Example 556: (S)-5-(Azetidin-2-ylmethoxy)-2-methyl-N-(1-(7-(5-methylfuran-2-yl)quinolin-5-yl)cyclopropyl)benzamide (Compound 646)

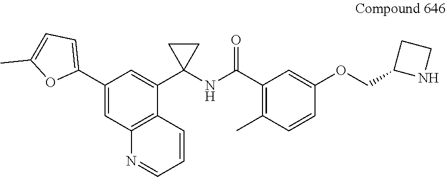 [SMILES: Cc1ccc(-c2cc(C3(NC(=O)c4cc(OC[C@@H]5CCN5)ccc4C)CC3)c3cccnc3c2)o1]

Compound 646 was synthesized according to an analogous procedure to the one described for compound 608. M+H⁺=468.3 (LCMS); ¹H NMR (400 MHZ, DMSO-d₆) δ 9.17 (s, 1H), 9.09 (br d, J=8.3 Hz, 1H), 9.00-8.64 (m, 3H), 8.24-8.05 (m, 2H), 7.60 (dd, J=4.3, 8.5 Hz, 1H), 7.15 (d, J=3.1 Hz, 1H), 7.10 (d, J=8.5 Hz, 1H), 6.91 (dd, J=2.7, 8.3 Hz, 1H), 6.70 (d, J=2.6 Hz, 1H), 6.32 (dd, J=0.9, 3.2 Hz, 1H), 4.74-4.57 (m, 1H), 4.31-4.18 (m, 1H), 4.17-4.06 (m, 1H), 4.00-3.87 (m, 1H), 3.86-3.77 (m, 1H), 2.43 (s, 3H), 2.40-2.21 (m, 2H), 1.98 (s, 3H), 1.39 (br s, 2H), 1.30 (br s, 2H).

### Example 557: (S)-5-(Azetidin-2-ylmethoxy)-2-methyl-N-(1-(7-(oxazol-2-yl)quinolin-5-yl)cyclopropyl)benzamide (Compound 694)

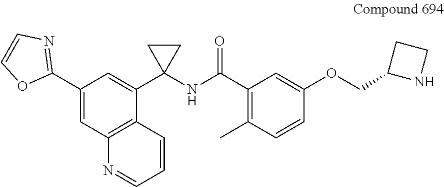 [SMILES: Cc1ccc(OC[C@@H]2CCN2)cc1C(=O)NC1(c2cc(-c3ncco3)cc3ncccc23)CC1]

Compound 694 was synthesized according to an analogous procedure to the one described for compound 608. M+H⁺=455.1 (LCMS); ¹H NMR (400 MHz, DMSO-d₆) δ 9.22 (s, 1H), 9.14 (d, J=7.9 Hz, 1H), 9.02 (dd, J=1.5, 4.1 Hz, 1H), 8.95-8.77 (m, 2H), 8.50 (s, 2H), 8.36 (s, 1H), 7.70 (dd, J=4.2, 8.6 Hz, 1H), 7.51 (s, 1H), 7.09 (d, J=8.5 Hz, 1H), 6.91 (dd, J=2.7, 8.3 Hz, 1H), 6.72 (d, J=2.8 Hz, 1H), 4.67-4.63 (m, 1H), 4.23 (dd, J=7.1, 11.2 Hz, 1H), 4.17-4.11 (m, 1H), 3.97-3.77 (m, 2H), 2.48-2.41 (m, 1H), 2.39-2.29 (m, 1H), 1.96 (s, 3H), 1.43 (br s, 2H), 1.29 (br s, 2H).

### Example 558: (S)-5-(Azetidin-2-ylmethoxy)-2-methyl-N-(1-(7-(2-methyloxazol-5-yl)quinolin-5-yl)cyclopropyl)benzamide (Compound 698)

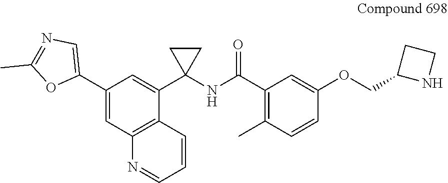 [SMILES: Cc1ncc(-c2cc(C3(NC(=O)c4cc(OC[C@@H]5CCN5)ccc4C)CC3)c3cccnc3c2)o1]

Compound 698 was synthesized according to an analogous procedure to the one described for compound 608. M+H⁺=469.2 (LCMS); ¹H NMR (400 MHZ, DMSO-d₆) δ 9.24-9.17 (m, 2H), 9.01 (dd, J=1.2, 4.3 Hz, 1H), 8.20 (s, 2H), 7.86 (s, 1H), 7.72 (dd, J=4.4, 8.6 Hz, 1H), 7.09 (d, J=8.5 Hz, 1H), 6.91 (dd, J=2.6, 8.4 Hz, 1H), 6.69 (d, J=2.8 Hz, 1H), 4.70-4.53 (m, 1H), 4.27-4.18 (m, 1H), 4.16-4.08 (m, 1H), 3.98-3.88 (m, 1H), 3.86-3.77 (m, 1H), 2.56 (s, 3H), 2.47-2.30 (m, 2H), 1.95 (s, 3H), 1.45-1.37 (m, 2H), 1.35-1.29 (m, 2H), 1.65-1.67 (m, 1H).

### Example 559: (S)-5-(Azetidin-2-ylmethoxy)-2-methyl-N-(1-(7-(5-methyl-1,3,4-oxadiazol-2-yl)quinolin-5-yl)cyclopropyl)benzamide (Compound 680)

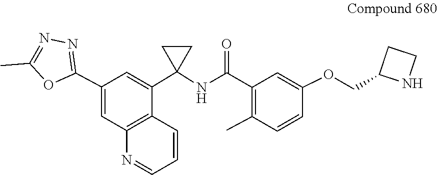 [SMILES: Cc1nnc(-c2cc(C3(NC(=O)c4cc(OC[C@@H]5CCN5)ccc4C)CC3)c3cccnc3c2)o1]

Compound 680 was synthesized according to an analogous procedure to the one described for compound 608. M+H⁺=470.3 (LCMS); ¹H NMR (400 MHZ, DMSO-d₆) δ 9.23 (s, 1H), 9.15 (d, J=8.3 Hz, 1H), 9.05 (dd, J=1.4, 4.1 Hz, 1H), 8.90-8.77 (m, 2H), 8.48 (s, 1H), 8.43 (d, J=1.6 Hz, 1H), 7.74 (dd, J=4.1, 8.6 Hz, 1H), 7.09 (d, J=8.5 Hz, 1H), 6.91 (dd, J=2.8, 8.4 Hz, 1H), 6.71 (d, J=2.6 Hz, 1H), 4.65 (br d, J=5.5 Hz, 1H), 4.28-4.19 (m, 1H), 4.18-4.11 (m, 1H), 4.00-3.87 (m, 2H), 2.66 (s, 3H), 2.46 (br s, 1H), 2.37-2.29 (m, 1H), 1.95 (s, 3H), 1.43 (br s, 2H), 1.29 (br s, 2H).

### Example 560: (S)—N-(1-(7-(1H-Pyrazol-3-yl)quinolin-5-yl)cyclopropyl)-5-(azetidin-2-ylmethoxy)-2-methylbenzamide (Compound 636)

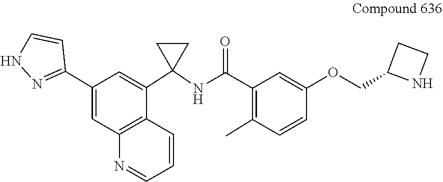 [SMILES: Cc1ccc(OC[C@@H]2CCN2)cc1C(=O)NC1(c2cc(-c3cc[nH]n3)cc3ncccc23)CC1]

Compound 636 was synthesized according to an analogous procedure to the one described for compound 608. M+H⁺=454.1 (LCMS); ¹H NMR (400 MHZ, DMSO-d₆) δ 9.23-9.15 (m, 2H), 8.99 (d, J=3.5 Hz, 1H), 8.88-8.71 (m, 2H), 8.43 (s, 1H), 8.35 (s, 1H), 7.86 (d, J=1.8 Hz, 1H), 7.67 (dd, J=4.5, 8.6 Hz, 1H), 7.10 (d, J=8.5 Hz, 1H), 6.97 (d, J=2.3 Hz, 1H), 6.91 (dd, J=2.5, 8.5 Hz, 1H), 6.71 (d, J=2.6 Hz, 1H), 4.71-4.60 (m, 1H), 4.23 (dd, J=6.9, 11.1 Hz, 1H), 4.16-4.11 (m, 1H), 3.96-3.86 (m, 2H), 2.44-2.29 (m, 2H), 1.97 (s, 3H), 1.41 (br s, 2H), 1.31 (br s, 2H).

### Example 561: (S)-5-(Azetidin-2-ylmethoxy)-2-methyl-N-(1-(7-(5-methyl-1H-pyrazol-3-yl)quinolin-5-yl)cyclopropyl)benzamide (Compound 635)

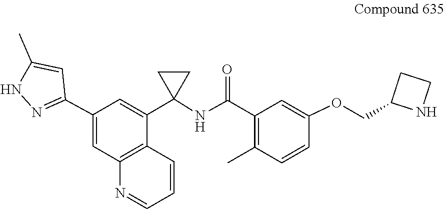 [SMILES: Cc1cc(-c2cc(C3(NC(=O)c4cc(OC[C@@H]5CCN5)ccc4C)CC3)c3cccnc3c2)n[nH]1]

Compound 635 was synthesized according to an analogous procedure to the one described for compound 608. M+H⁺=468.2 (LCMS); ¹H NMR (400 MHZ, DMSO-d₆) δ 9.40 (d, J=8.5 Hz, 1H), 9.22 (s, 1H), 9.09 (dd, J=1.3, 4.8 Hz, 1H), 8.47 (d, J=1.4 Hz, 1H), 8.35 (s, 1H), 7.83 (dd, J=4.8, 8.6 Hz, 1H), 7.09 (d, J=8.6 Hz, 1H), 6.91 (dd, J=2.7, 8.4 Hz, 1H), 6.76-6.68 (m, 2H), 4.65 (dq, J=3.2, 7.8 Hz, 1H), 4.28-4.19 (m, 1H), 4.17-4.10 (m, 1H), 3.98-3.88 (m, 1H), 3.82 (dt, J=6.3, 10.0 Hz, 1H), 2.47-2.41 (m, 1H), 2.39-2.29 (m, 4H), 1.96 (s, 3H), 1.43 (br s, 2H), 1.32 (br s, 2H).

### Example 562: (S)-5-(Azetidin-2-ylmethoxy)-2-methyl-N-(1-(7-(1-methyl-1H-pyrazol-5-yl)quinolin-5-yl)cyclopropyl)benzamide (Compound 649)

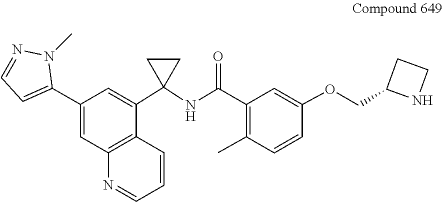 [SMILES: Cc1ccc(OC[C@@H]2CCN2)cc1C(=O)NC1(c2cc(-c3ccnn3C)cc3ncccc23)CC1]

Compound 649 was synthesized according to an analogous procedure to the one described for compound 608. M+H⁺=468.2 (LCMS); ¹H NMR (400 MHZ, DMSO-d₆) δ 9.22 (s, 1H), 9.18-9.11 (m, 1H), 9.05-8.98 (m, 1H), 8.95-8.77 (m, 2H), 8.15-8.02 (m, 2H), 7.78-7.65 (m, 1H), 7.61-7.52 (m, 1H), 7.16-7.05 (m, 1H), 6.97-6.87 (m, 1H), 6.78-6.69 (m, 1H), 6.67-6.59 (m, 1H), 4.76-4.58 (m, 1H), 4.28-4.19 (m, 1H), 4.17-4.09 (m, 1H), 4.00 (s, 3H), 3.97-3.81 (m, 2H), 2.47-2.41 (m, 1H), 2.39-2.31 (m, 1H), 1.97 (s, 3H), 1.40 (br s, 2H), 1.32 (br s, 2H).

### Example 563: (S)-5-(Azetidin-2-ylmethoxy)-2-methyl-N-(1-(7-(1-methyl-1H-pyrazol-3-yl)quinolin-5-yl)cyclopropyl)benzamide (Compound 634)

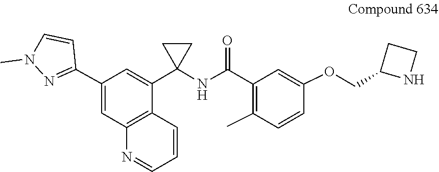 [SMILES: Cc1ccc(OC[C@@H]2CCN2)cc1C(=O)NC1(c2cc(-c3ccn(C)n3)cc3ncccc23)CC1]

Compound 634 was synthesized according to an analogous procedure to the one described for compound 608. M+H⁺=468.1 (LCMS); ¹H NMR (400 MHZ, DMSO-d₆) δ 9.21-9.14 (m, 2H), 8.98 (dd, J=1.4, 4.4 Hz, 1H), 8.88-8.78 (m, 1H), 8.43 (d, J=1.5 Hz, 1H), 8.31 (s, 1H), 7.85 (d, J=2.1 Hz, 1H), 7.65 (dd, J=4.4, 8.5 Hz, 1H), 7.10 (d, J=8.5 Hz, 1H), 6.95 (d, J=2.3 Hz, 1H), 6.91 (dd, J=2.7, 8.4 Hz, 1H), 6.71 (d, J=2.7 Hz, 1H), 4.71-4.60 (m, 1H), 4.28-4.19 (m, 1H), 4.17-4.10 (m, 1H), 3.97 (s, 3H), 3.88-3.78 (m, 2H), 2.45-2.30 (m, 2H), 1.98 (s, 3H), 1.41 (br s, 2H), 1.29 (br s, 2H).

### Example 564: (S)-5-(Azetidin-2-ylmethoxy)-N-(1-(7-(1-(difluoromethyl)-1H-pyrazol-3-yl)quinolin-5-yl)cyclopropyl)-2-methylbenzamide (Compound 656)

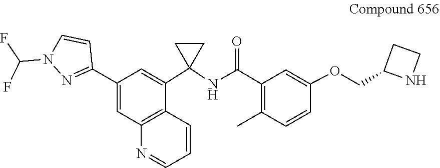 [SMILES: Cc1ccc(OC[C@@H]2CCN2)cc1C(=O)NC1(c2cc(-c3ccn(C(F)F)n3)cc3ncccc23)CC1]

Compound 656 was synthesized according to an analogous procedure to the one described for compound 608. M+H⁺=504.1 (LCMS); ¹H NMR (400 MHZ, DMSO-d₆) δ 9.19 (s, 1H), 9.14 (d, J=8.4 Hz, 1H), 8.98 (dd, J=1.5, 4.3 Hz, 1H), 8.88-8.73 (m, 2H), 8.48-8.43 (m, 2H), 8.40 (d, J=2.8 Hz, 1H), 8.13-7.80 (m, 1H), 7.65 (dd, J=4.2, 8.6 Hz, 1H), 7.32 (d, J=2.8 Hz, 1H), 7.10 (d, J=8.5 Hz, 1H), 6.91 (dd, J=2.8, 8.4 Hz, 1H), 6.70 (d, J=2.8 Hz, 1H), 4.69-4.60 (m, 1H), 4.23 (dd, J=7.1, 11.3 Hz, 2H), 4.13 (br dd, J=3.3, 11.1 Hz, 2H), 2.45-2.28 (m, 2H), 1.97 (s, 3H), 1.41 (br s, 2H), 1.30 (br s, 2H).

### Example 565: (S)-5-(Azetidin-2-ylmethoxy)-2-methyl-N-(1-(7-(1-phenyl-1H-pyrazol-3-yl)quinolin-5-yl)cyclopropyl)benzamide (Compound 664)

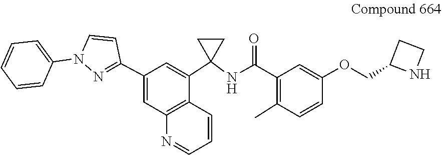 [SMILES: Cc1ccc(OC[C@@H]2CCN2)cc1C(=O)NC1(c2cc(-c3ccn(-c4ccccc4)n3)cc3ncccc23)CC1]

Compound 664 was synthesized according to an analogous procedure to the one described for compound 608. M+H⁺=530.3 (LCMS); ¹H NMR (400 MHZ, DMSO-d₆) δ 9.24-9.15 (m, 2H), 9.00 (dd, J=1.4, 4.3 Hz, 1H), 8.96-8.72 (m, 2H), 8.68 (d, J=2.5 Hz, 1H), 8.53 (d, J=1.6 Hz, 1H), 8.48 (s, 1H), 8.04-7.95 (m, 2H), 7.67 (dd, J=4.3, 8.6 Hz, 1H), 7.58 (t, J=8.0 Hz, 2H), 7.43-7.34 (m, 1H), 7.32 (d, J=2.6 Hz, 1H), 7.10 (d, J=8.5 Hz, 1H), 6.91 (dd, J=2.6, 8.4 Hz, 1H), 6.71 (d, J=2.6 Hz, 1H), 4.65 (br d, J=3.4 Hz, 1H), 4.27-4.19 (m, 1H), 4.17-4.10 (m, 1H), 4.00-3.89 (m, 2H), 2.45-2.28 (m, 2H), 1.99 (s, 3H), 1.44 (br s, 2H), 1.34 (br s, 2H).

### Example 566: (S)—N-(1-(7-(1H-Pyrazol-4-yl)quinolin-5-yl)cyclopropyl)-5-(azetidin-2-ylmethoxy)-2-methylbenzamide (Compound 642)

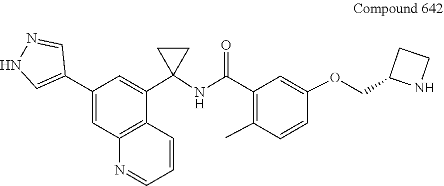 [SMILES: Cc1ccc(OC[C@@H]2CCN2)cc1C(=O)NC1(c2cc(-c3cn[nH]c3)cc3ncccc23)CC1]

Compound 642 was synthesized according to an analogous procedure to the one described for compound 608. M+H⁺=454.2 (LCMS); ¹H NMR (400 MHZ, DMSO-d₆) δ 9.24-9.08 (m, 2H), 8.98-8.91 (m, 1H), 8.85-8.69 (m, 2H), 8.32 (br s, 2H), 8.21-8.10 (m, 2H), 7.67-7.54 (m, 1H), 7.17-7.02 (m, 1H), 6.96-6.87 (m, 1H), 6.75-6.65 (m, 1H), 4.72-4.58 (m, 1H), 4.25 (s, 1H), 4.17-4.11 (m, 1H), 3.94-3.90 (m, 1H), 3.86-3.80 (m, 1H), 2.47-2.42 (m, 1H), 2.33 (br s, 1H), 1.98 (s, 3H), 1.43-1.32 (m, 4H).

### Example 567: (S)-5-(Azetidin-2-ylmethoxy)-2-methyl-N-(1-(7-(1-methyl-1H-pyrazol-4-yl)quinolin-5-yl)cyclopropyl)benzamide (Compound 665)

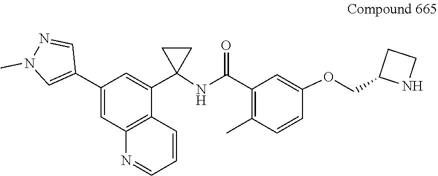 [SMILES: Cc1ccc(OC[C@@H]2CCN2)cc1C(=O)NC1(c2cc(-c3cnn(C)c3)cc3ncccc23)CC1]

Compound 665 was synthesized according to an analogous procedure to the one described for compound 608. M+H⁺=468.2 (LCMS); ¹H NMR (400 MHz, DMSO-d₆) δ 9.19-9.07 (m, 2H), 8.95 (dd, J=1.4, 4.4 Hz, 1H), 8.93-8.75 (m, 2H), 8.44 (s, 1H), 8.13-8.08 (m, 3H), 7.62 (dd, J=4.4, 8.5 Hz, 1H), 7.10 (d, J=8.5 Hz, 1H), 6.91 (dd, J=2.8, 8.4 Hz, 1H), 6.70 (d, J=2.8 Hz, 1H), 4.70-4.60 (m, 1H), 4.23 (dd, J=7.1, 11.2 Hz, 2H), 4.13 (br dd, J=3.3, 11.3 Hz, 2H), 3.93 (s, 3H), 2.48-2.29 (m, 2H), 1.98 (s, 3H), 1.39 (br s, 2H), 1.36-1.29 (m, 2H).

### Example 568: (S)-5-(Azetidin-2-ylmethoxy)-N-(1-(7-(benzo[b]thiophen-2-yl)quinolin-5-yl)cyclopropyl)-2-methylbenzamide (Compound 695)

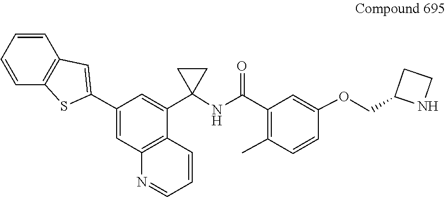 [SMILES: Cc1ccc(OC[C@@H]2CCN2)cc1C(=O)NC1(c2cc(-c3cc4ccccc4s3)cc3ncccc23)CC1]

Compound 695 was synthesized according to an analogous procedure to the one described for compound 608. M+H⁺=520.3 (LCMS); ¹H NMR (400 MHZ, DMSO-d₆) δ 9.21 (s, 1H), 9.09 (d, J=7.7 Hz, 1H), 8.98 (dd, J=1.3, 4.1 Hz, 1H), 8.89-8.67 (m, 2H), 8.34 (d, J=1.9 Hz, 1H), 8.28-8.14 (m, 2H), 8.09-8.01 (m, 1H), 7.96 (dd, J=1.8, 6.5 Hz, 1H), 7.64 (dd, J=4.2, 8.6 Hz, 1H), 7.50-7.37 (m, 2H), 7.11 (d, J=8.5 Hz, 1H), 6.97-6.87 (m, 1H), 6.71 (d, J=2.8 Hz, 1H), 4.69-4.59 (m, 1H), 4.28-4.20 (m, 1H), 4.18-4.11 (m, 1H), 3.94-3.88 (m, 1H), 3.83 (br dd, J=5.3, 10.5 Hz, 1H), 2.46-2.25 (m, 2H), 2.00 (s, 3H), 1.42 (br s, 2H), 1.37 (br s, 2H).

### Example 569: (S)-5-(Azetidin-2-ylmethoxy)-N-(1-(7-(benzo[d]oxazol-2-yl)quinolin-5-yl)cyclopropyl)-2-methylbenzamide (Compound 711)

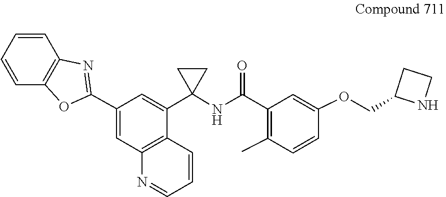 [SMILES: Cc1ccc(OC[C@@H]2CCN2)cc1C(=O)NC1(c2cc(-c3nc4ccccc4o3)cc3ncccc23)CC1]

Compound 711 was synthesized according to an analogous procedure to the one described for compound 608. M+H⁺=505.2 (LCMS); ¹H NMR (400 MHZ, DMSO-d₆) δ 9.26 (s, 1H), 9.18 (d, J=8.3 Hz, 1H), 9.07 (dd, J=1.5, 4.1 Hz, 1H), 8.94-8.76 (m, 2H), 8.73 (s, 1H), 8.69 (d, J=1.8 Hz, 1H), 7.96-7.88 (m, 2H), 7.75 (dd, J=4.1, 8.5 Hz, 1H), 7.49 (dquin, J=1.5, 7.3 Hz, 2H), 7.10 (d, J=8.5 Hz, 1H), 6.91 (dd, J=2.6, 8.4 Hz, 1H), 6.73 (d, J=2.6 Hz, 1H), 4.71-4.58 (m, 1H), 4.28-4.19 (m, 1H), 4.18-4.11 (m, 1H), 3.96-3.77 (m, 2H), 2.48-2.40 (m, 1H), 2.38-2.28 (m, 1H), 1.98 (s, 3H), 1.46 (br s, 2H), 1.34 (br s, 2H).

### Example 570: (S)-5-(Azetidin-2-ylmethoxy)-N-(1-(7-(cyclohex-1-en-1-yl)quinolin-5-yl)cyclopropyl)-2-methylbenzamide (Compound 662)

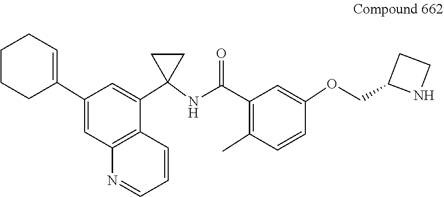 [SMILES: Cc1ccc(OC[C@@H]2CCN2)cc1C(=O)NC1(c2cc(C3=CCCCC3)cc3ncccc23)CC1]

Compound 662 was synthesized according to an analogous procedure to the one described for compound 608. M+H⁺=468.2 (LCMS); ¹H NMR (400 MHZ, DMSO-d₆) δ 9.24-9.11 (m, 2H), 9.02-8.96 (m, 1H), 8.95-8.76 (m, 2H), 8.11 (d, J=1.6 Hz, 1H), 7.89 (s, 1H), 7.68 (dd, J=4.5, 8.5 Hz, 1H), 7.10 (d, J=8.5 Hz, 1H), 6.91 (dd, J=2.7, 8.3 Hz, 1H), 6.69 (d, J=2.8 Hz, 1H), 6.54 (br s, 1H), 4.72-4.60 (m, 1H), 4.27-4.19 (m, 1H), 4.17-4.10 (m, 1H), 4.02-3.81 (m, 4H), 2.44-2.18 (m, 4H), 1.97 (s, 3H), 1.86-1.77 (m, 2H), 1.72-1.63 (m, 2H), 1.37 (br s, 2H), 1.28 (br s, 2H).

### Example 571: (S)-5-(Azetidin-2-ylmethoxy)-N-(1-(7-(4-fluorophenyl)quinolin-5-yl)cyclopropyl)-2-methylbenzamide (Compound 675)

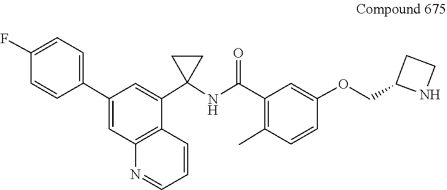 [SMILES: Cc1ccc(OC[C@@H]2CCN2)cc1C(=O)NC1(c2cc(-c3ccc(F)cc3)cc3ncccc23)CC1]

Compound 675 was synthesized according to an analogous procedure to the one described for compound 608. M+H⁺=482.1 (LCMS); ¹H NMR (400 MHZ, DMSO-d₆) δ 9.23-9.12 (m, 2H), 8.99 (br d, J=3.9 Hz, 1H), 8.90-8.74 (m, 2H), 8.20 (s, 2H), 7.94 (dd, J=5.5, 8.6 Hz, 2H), 7.66 (dd, J=4.2, 8.5 Hz, 1H), 7.40 (t, J=8.8 Hz, 2H), 7.10 (d, J=8.4 Hz, 1H), 6.91 (dd, J=2.4, 8.4 Hz, 1H), 6.70 (d, J=2.4 Hz, 1H), 4.65 (br d, J=4.3 Hz, 1H), 4.23 (br dd, J=7.2, 11.1 Hz, 1H), 4.17-4.09 (m, 1H), 3.95-3.89 (m, 1H), 3.85-3.79 (m, 1H), 2.47-2.28 (m, 2H), 1.98 (s, 3H), 1.38 (br d, J=15.4 Hz, 4H).

### Example 572: (S)-5-(Azetidin-2-ylmethoxy)-N-(1-(7-(3-chloro-4-fluorophenyl)quinolin-5-yl)cyclopropyl)-2-methylbenzamide (Compound 666)

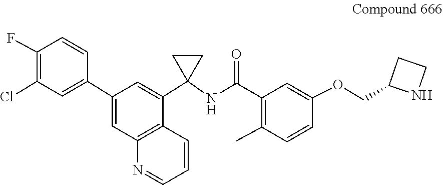 [SMILES: Cc1ccc(OC[C@@H]2CCN2)cc1C(=O)NC1(c2cc(-c3ccc(F)c(Cl)c3)cc3ncccc23)CC1]

Compound 666 was synthesized according to an analogous procedure to the one described for compound 608. M+H⁺=516.2 (LCMS); ¹H NMR (400 MHZ, DMSO-d₆) δ 9.23-9.13 (m, 2H), 9.00 (dd, J=1.5, 4.3 Hz, 1H), 8.97-8.76 (m, 2H), 8.26 (d, J=1.4 Hz, 1H), 8.20 (d, J=1.9 Hz, 1H), 8.13 (dd, J=2.3, 7.1 Hz, 1H), 7.92 (ddd, J=2.4, 4.7, 8.6 Hz, 1H), 7.68 (dd, J=4.3, 8.5 Hz, 1H), 7.61 (t, J=8.9 Hz, 1H), 7.09 (d, J=8.5 Hz, 1H), 6.91 (dd, J=2.6, 8.4 Hz, 1H), 6.70 (d, J=2.6 Hz, 1H), 4.65 (br d, J=6.3 Hz, 1H), 4.23 (dd, J=7.2, 11.2 Hz, 1H), 4.17-4.09 (m, 1H), 3.97-3.78 (m, 2H), 2.48-2.26 (m, 2H), 1.98 (s, 3H), 1.40 (br s, 4H).

### Example 573: (S)-5-(Azetidin-2-ylmethoxy)-2-methyl-N-(1-(7-(6-oxo-1,6-dihydropyridin-3-yl)quinolin-5-yl)cyclopropyl)benzamide (Compound 674)

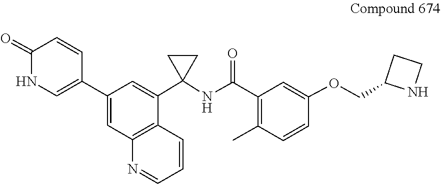 [SMILES: Cc1ccc(OC[C@@H]2CCN2)cc1C(=O)NC1(c2cc(-c3ccc(=O)[nH]c3)cc3ncccc23)CC1]

Compound 674 was synthesized according to an analogous procedure to the one described for compound 608. M+H⁺=481.2 (LCMS); ¹H NMR (400 MHZ, DMSO-d₆) δ 9.19-9.09 (m, 2H), 8.97 (dd, J=1.3, 4.3 Hz, 1H), 8.95-8.71 (m, 2H), 8.16-7.94 (m, 4H), 7.64 (dd, J=4.4, 8.5 Hz, 1H), 7.10 (d, J=8.5 Hz, 1H), 6.91 (dd, J=2.7, 8.3 Hz, 1H), 6.69 (d, J=2.6 Hz, 1H), 6.53 (d, J=9.5 Hz, 1H), 4.65 (br d, J=4.8 Hz, 1H), 4.30-4.18 (m, 1H), 4.17-4.08 (m, 1H), 3.99-3.73 (m, 2H), 2.47-2.42 (m, 1H), 2.38-2.28 (m, 1H), 1.98 (s, 3H), 1.37 (br s, 4H).

### Example 574: (S)-5-(Azetidin-2-ylmethoxy)-2-methyl-N-(1-(7-(pyridin-3-yl)quinolin-5-yl)cyclopropyl)benzamide (Compound 637)

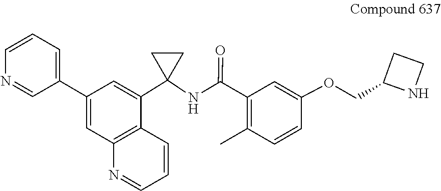 [SMILES: Cc1ccc(OC[C@@H]2CCN2)cc1C(=O)NC1(c2cc(-c3cccnc3)cc3ncccc23)CC1]

Compound 637 was synthesized according to an analogous procedure to the one described for compound 608. M+H⁺=465.2 (LCMS); ¹H NMR (400 MHZ, DMSO-d₆) δ 9.28-9.16 (m, 3H), 9.07-9.01 (m, 1H), 8.78 (dd, J=1.3, 5.1 Hz, 1H), 8.63-8.56 (m, 1H), 8.38 (d, J=1.6 Hz, 1H), 8.27 (d, J=1.6 Hz, 1H), 7.82 (dd, J=5.2, 7.8 Hz, 1H), 7.73 (dd, J=4.3, 8.5 Hz, 1H), 7.10 (d, J=8.5 Hz, 1H), 6.91 (dd, J=2.8, 8.4 Hz, 1H), 6.70 (d, J=2.6 Hz, 1H), 4.65 (br dd, J=2.7, 8.0 Hz, 1H), 4.27-4.18 (m, 1H), 4.16-4.09 (m, 1H), 3.97-3.77 (m, 2H), 2.41-2.25 (m, 2H), 1.96 (s, 3H), 1.41 (br s, 4H).

### Example 575: (S)-5-(Azetidin-2-ylmethoxy)-2-methyl-N-(1-(7-(pyrimidin-2-yl)quinolin-5-yl)cyclopropyl)benzamide (Compound 682)

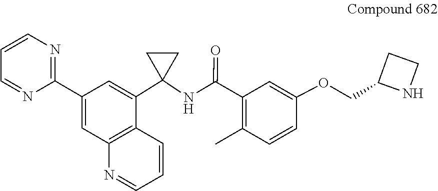 [SMILES: Cc1ccc(OC[C@@H]2CCN2)cc1C(=O)NC1(c2cc(-c3ncccn3)cc3ncccc23)CC1]

Compound 682 was synthesized according to an analogous procedure to the one described for compound 608. M+H⁺=466.1 (LCMS); ¹H NMR (400 MHZ, DMSO-d₆) δ=9.26-9.13 (m, 2H), 9.08-9.01 (m, 3H), 9.00-8.92 (m, 2H), 8.90-8.71 (m, 2H), 7.71 (dd, J=4.2, 8.6 Hz, 1H), 7.57 (t, J=4.8 Hz, 1H), 7.10 (d, J=8.5 Hz, 1H), 6.91 (dd, J=2.6, 8.3 Hz, 1H), 6.71 (d, J=2.6 Hz, 1H), 4.71-4.59 (m, 1H), 4.27-4.19 (m, 1H), 4.17-4.10 (m, 1H), 4.18-4.10 (m, 1H), 3.96-3.88 (m, 1H), 3.86-3.78 (m, 1H), 2.47-2.29 (m, 2H), 1.98 (s, 3H), 1.45 (br s, 2H), 1.30 (br s, 2H).

### Example 576: (S)-5-(Azetidin-2-ylmethoxy)-2-methyl-N-(1-(7-(2-(piperidin-1-yl)pyrimidin-5-yl)quinolin-5-yl)cyclopropyl)benzamide (Compound 688)

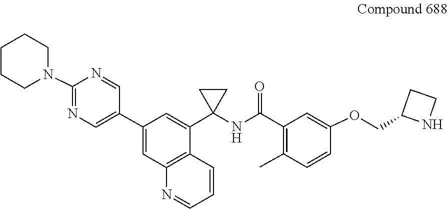 [SMILES: Cc1ccc(OC[C@@H]2CCN2)cc1C(=O)NC1(c2cc(-c3cnc(N4CCCCC4)nc3)cc3ncccc23)CC1]

Compound 688 was synthesized according to an analogous procedure to the one described for compound 608. M+H⁺=549.5 (LCMS); ¹H NMR (400 MHZ, DMSO-d₆) δ 9.21-9.10 (m, 2H), 8.99-8.96 (m, 1H), 8.90 (s, 2H), 8.88-8.70 (m, 2H), 8.18 (dd, J=1.6, 19.5 Hz, 2H), 7.64 (dd, J=4.4, 8.5 Hz, 1H), 7.10 (d, J=8.5 Hz, 1H), 6.91 (dd, J=2.7, 8.3 Hz, 1H), 6.70 (d, J=2.8 Hz, 1H), 4.70-4.61 (m, 1H), 4.21 (br d, J=7.3 Hz, 1H), 4.15 (br d, J=3.4 Hz, 1H), 3.97-3.88 (m, 2H), 3.86-3.83 (m, 4H), 2.48-2.40 (m, 1H), 2.40-2.27 (m, 1H), 1.98 (s, 3H), 1.72-1.64 (m, 2H), 1.57 (br d, J=3.9 Hz, 4H), 1.39 (br s, 4H).

### Example 577: (S)-5-(Azetidin-2-ylmethoxy)-N-(1-(7-(imidazo[1,2-a]pyridin-6-yl)quinolin-5-yl)cyclopropyl)-2-methylbenzamide (Compound 686)

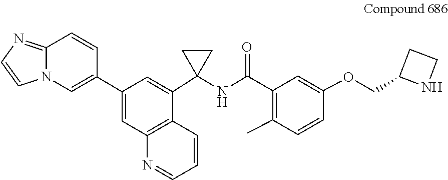 [SMILES: Cc1ccc(OC[C@@H]2CCN2)cc1C(=O)NC1(c2cc(-c3ccc4nccn4c3)cc3ncccc23)CC1]

Compound 686 was synthesized according to an analogous procedure to the one described for compound 608. M+H⁺=504.3 (LCMS); ¹H NMR (400 MHZ, DMSO-d₆) δ 9.54 (s, 1H), 9.25 (s, 1H), 9.12 (d, J=8.7 Hz, 1H), 9.02 (dd, J=1.5, 4.0 Hz, 1H), 8.97-8.77 (m, 2H), 8.51 (dd, J=1.3, 9.6 Hz, 1H), 8.43-8.37 (m, 2H), 8.30-8.22 (m, 2H), 8.15-8.09 (m, 1H), 7.74-7.66 (m, 1H), 7.12 (s, 1H), 6.95-6.89 (m, 1H), 6.72-6.67 (m, 1H), 4.66 (br d, J=6.1 Hz, 1H), 4.27-4.20 (m, 1H), 4.16-4.10 (m, 1H), 3.95-3.90 (m, 1H), 3.85-3.81 (m, 1H), 2.47-2.41 (m, 1H), 2.39-2.31 (m, 1H), 1.97 (s, 3H), 1.45-1.36 (m, 4H).

### Example 578: (S)—N-(1-(7-([1,2,4]Triazolo[1,5-a]pyridin-6-yl)quinolin-5-yl)cyclopropyl)-5-(azetidin-2-ylmethoxy)-2-methylbenzamide (Compound 685)

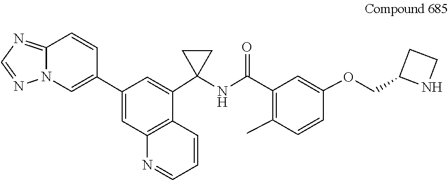 [SMILES: Cc1ccc(OC[C@@H]2CCN2)cc1C(=O)NC1(c2cc(-c3ccc4ncnn4c3)cc3ncccc23)CC1]

Compound 685 was synthesized according to an analogous procedure to the one described for compound 608. M+H⁺=505.3 (LCMS); ¹H NMR (400 MHZ, DMSO-d₆) δ 9.60 (s, 1H), 9.20 (s, 1H), 9.16 (d, J=8.3 Hz, 1H), 9.01 (dd, J=1.3, 4.2 Hz, 1H), 8.94-8.73 (m, 2H), 8.61 (s, 1H), 8.39 (d, J=1.4 Hz, 1H), 8.30 (d, J=1.8 Hz, 1H), 8.27-8.23 (m, 1H), 8.08-8.01 (m, 1H), 7.71-7.64 (m, 1H), 7.10 (d, J=8.5 Hz, 1H), 6.91 (dd, J=2.6, 8.4 Hz, 1H), 6.71 (d, J=2.6 Hz, 1H), 4.68-4.62 (m, 1H), 4.26-4.20 (m, 1H), 4.16-4.10 (m, 1H), 3.93-3.79 (m, 2H), 2.49-2.41 (m, 1H), 2.38-2.27 (m, 1H), 1.98 (s, 3H), 1.43 (br d, J=14.2 Hz, 4H).

### Example 579: (S)—N-(1-(7-Ethynyl-2-methylquinolin-5-yl)cyclopropyl)-2-methyl-5-((1-methylazetidin-2-yl)methoxy)benzamide (Compound 822)

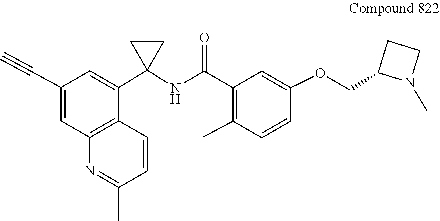 [SMILES: C#Cc1cc(C2(NC(=O)c3cc(OC[C@@H]4CCN4C)ccc3C)CC2)c2ccc(C)nc2c1]

Compound 822 was synthesized according to an analogous procedure to the one described for compound 620. M+H⁺=440.2 (LCMS); ¹H NMR (400 MHZ, DMSO-d₆) δ 10.44-10.12 (m, 1H), 9.33-9.16 (m, 2H), 8.11 (s, 1H), 7.92 (s, 1H), 7.80 (br d, J=8.1 Hz, 1H), 7.15-7.05 (m, 1H), 6.99-6.90 (m, 1H), 6.82-6.68 (m, 1H), 4.62 (br s, 2H), 4.31-4.20 (m, 2H), 4.03-3.98 (m, 1H), 3.88-3.85 (m, 1H), 2.86-2.78 (m, 6H), 2.41-2.27 (m, 2H), 1.96 (s, 3H), 1.38 (br s, 2H), 1.27 (br s, 2H).

### Example 580: 2-Methyl-5-(2-(methylamino)ethoxy)-N-(1-(7-vinylquinolin-5-yl)cyclopropyl)benzamide (Compound 615)

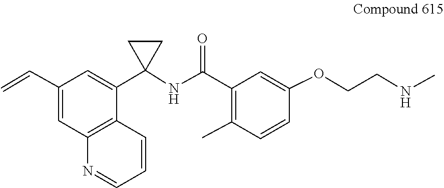 [SMILES: C=Cc1cc(C2(NC(=O)c3cc(OCCNC)ccc3C)CC2)c2cccnc2c1]

Compound 615 was synthesized according to an analogous procedure to the one described for compound 630. M+H⁺=402.2 (LCMS); ¹H NMR (400 MHZ, DMSO-d₆) δ 9.47-9.42 (m, 1H), 9.27-9.24 (m, 1H), 9.18-9.03 (m, 1H), 8.25-8.15 (m, 1H), 8.12-8.00 (m, 1H), 7.96-7.79 (m, 1H), 7.12-6.99 (m, 2H), 6.93-6.87 (m, 1H), 6.71-6.67 (m, 1H), 6.19 (d, J=17.5 Hz, 1H), 5.62 (d, J=11.0 Hz, 1H), 4.13 (t, J=5.0 Hz, 2H), 3.25 (t, J=4.9 Hz, 2H), 2.58 (s, 3H), 1.95 (s, 3H), 1.40 (br s, 2H), 1.33 (br s, 2H).

### Example 581: 2-Methyl-5-(2-(methylamino)ethoxy)-N-(1-(7-(prop-1-en-2-yl)quinolin-5-yl)cyclopropyl)benzamide (Compound 616)

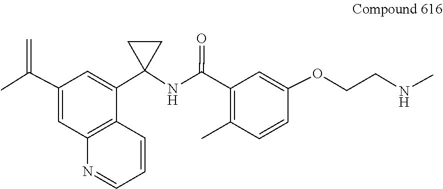 [SMILES: C=C(C)c1cc(C2(NC(=O)c3cc(OCCNC)ccc3C)CC2)c2cccnc2c1]

Compound 616 was synthesized according to an analogous procedure to the one described for compound 630. M+H⁺=416.2 (LCMS); ¹H NMR (400 MHZ, DMSO-d₆) δ 9.62-9.50 (m, 1H), 9.34-9.26 (m, 1H), 9.22-9.14 (m, 1H), 8.31-8.25 (m, 1H), 8.19-8.13 (m, 1H), 8.02-7.93 (m, 1H), 7.14-7.06 (m, 1H), 6.95-6.86 (m, 1H), 6.77-6.65 (m, 1H), 5.84 (s, 1H), 5.49 (s, 1H), 4.15 (t, J=5.0 Hz, 2H), 3.26 (br t, J=4.9 Hz, 2H), 2.58 (s, 3H), 2.27 (s, 3H), 1.97 (s, 3H), 1.42 (br s, 2H), 1.34 (br s, 2H).

### Example 582: 2-Methyl-N-(1-(7-(1-methyl-1H-pyrazol-5-yl)quinolin-5-yl)cyclopropyl)-5-(2-(methylamino)ethoxy)benzamide (Compound 639)

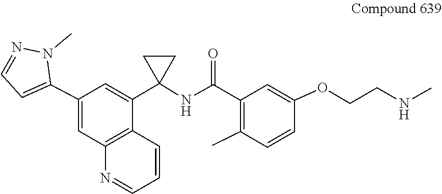 [SMILES: CNCCOc1ccc(C)c(C(=O)NC2(c3cc(-c4ccnn4C)cc4ncccc34)CC2)c1]

Compound 639 was synthesized according to an analogous procedure to the one described for compound 630. M+H⁺=456.2 (LCMS); ¹H NMR (400 MHZ, DMSO-d₆) δ 9.22 (s, 1H), 9.15 (br d, J=8.5 Hz, 1H), 9.02 (d, J=4.1 Hz, 1H), 8.11 (d, J=1.0 Hz, 1H), 8.06 (d, J=1.5 Hz, 1H), 7.71 (dd, J=4.2, 8.6 Hz, 1H), 7.57 (d, J=1.9 Hz, 1H), 7.09 (d, J=8.4 Hz, 1H), 6.90 (dd, J=2.8, 8.4 Hz, 1H), 6.68 (d, J=2.5 Hz, 1H), 6.64 (d, J=1.8 Hz, 1H), 4.12 (t, J=4.9 Hz, 2H), 4.00 (s, 3H), 3.27 (br t, J=4.7 Hz, 2H), 2.60 (s, 3H), 1.97 (s, 3H), 1.39 (br s, 2H), 1.32 (br s, 2H).

### Example 583: 2-Methyl-N-(1-(7-(1-methyl-1H-pyrazol-3-yl)quinolin-5-yl)cyclopropyl)-5-(2-(methylamino)ethoxy)benzamide (Compound 647)

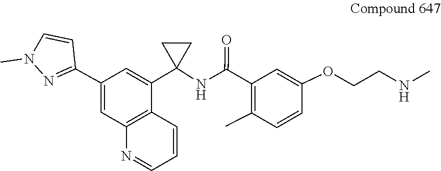

Compound 647 was synthesized according to an analogous procedure to the one described for compound 630. M+H⁺=456.1 (LCMS); ¹H NMR (400 MHZ, DMSO-d₆) δ 9.46-9.32 (m, 1H), 9.26 (s, 1H), 9.08 (br d, J=3.5 Hz, 1H), 8.76 (br dd, J=1.3, 7.4 Hz, 2H), 8.48 (s, 1H), 8.39 (s, 1H), 7.88 (d, J=2.3 Hz, 1H), 7.84-7.75 (m, 1H), 7.10 (d, J=8.5 Hz, 1H), 6.97 (d, J=2.3 Hz, 1H), 6.91 (dd, J=2.8, 8.4 Hz, 1H), 6.71 (d, J=2.6 Hz, 1H), 4.15 (t, J=4.9 Hz, 2H), 3.99 (s, 3H), 3.27 (br d, J=5.3 Hz, 2H), 2.59 (t, J=5.4 Hz, 3H), 1.98 (s, 3H), 1.43 (br s, 2H), 1.32 (br s, 2H).

### Example 584: (S)—N-(1-(2,7-Dimethylquinolin-5-yl)cyclopropyl)-2-methyl-5-((1-methylazetidin-2-yl)methoxy)benzamide (Compound 802)

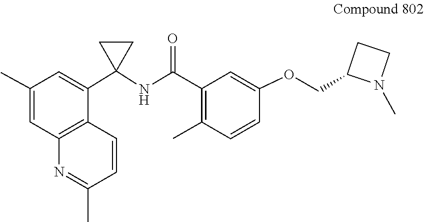

Compound 802 was synthesized according to an analogous procedure to the one described for compound 653. M+H⁺=430.2 (LCMS); ¹H NMR (400 MHZ, DMSO-d₆) δ 9.48 (d, J=8.8 Hz, 1H), 9.22 (s, 1H), 7.96-7.79 (m, 3H), 7.09 (br d, J=8.5 Hz, 1H), 6.92 (dd, J=2.4, 8.4 Hz, 1H), 6.72 (d, J=2.4 Hz, 1H), 4.66-4.56 (m, 1H), 4.21 (br d, J=5.1 Hz, 2H), 4.03 (dt, J=4.6, 9.5 Hz, 1H), 3.86 (br d, J=9.6 Hz, 1H), 2.85 (d, J=16.0 Hz, 6H), 2.60 (s, 3H), 2.43-2.28 (m, 2H), 1.94 (s, 3H), 1.39 (br s, 2H), 1.27 (br s, 2H).

### Example 585: (S)-2-Methyl-5-((1-methylpyrrolidin-2-yl)methoxy)-N-(1-(7-vinylquinolin-5-yl)cyclopropyl)benzamide (Compound 717)

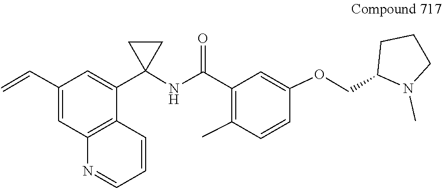 [SMILES: C=Cc1cc(C2(NC(=O)c3cc(OC[C@@H]4CCCN4C)ccc3C)CC2)c2cccnc2c1]

Compound 717 was synthesized according to an analogous procedure to the one described for compound 653. M+H⁺=442.1 (LCMS); ¹H NMR (400 MHZ, DMSO-d₆) δ 9.64-9.49 (m, 1H), 9.36-9.28 (m, 1H), 9.18 (br s, 1H), 8.25 (s, 1H), 8.13 (br s, 1H), 8.04-7.84 (m, 1H), 7.13-6.98 (m, 2H), 6.92 (dd, J=2.7, 8.3 Hz, 1H), 6.74 (d, J=2.3 Hz, 1H), 6.23 (d, J=17.6 Hz, 1H), 5.66 (br d, J=10.8 Hz, 1H), 4.29-4.16 (m, 2H), 3.80-3.64 (m, 1H), 3.60-3.55 (m, 1H), 3.14-3.04 (m, 1H), 2.89 (s, 3H), 2.28-2.14 (m, 1H), 2.06-1.85 (m, 5H), 1.81-1.72 (m, 1H), 1.43 (br s, 2H), 1.34 (br s, 2H).

### Example 586: (S)-2-Methyl-N-(1-(2-methyl-7-vinylquinolin-5-yl)cyclopropyl)-5-((1-methylazetidin-2-yl)methoxy)benzamide (Compound 813)

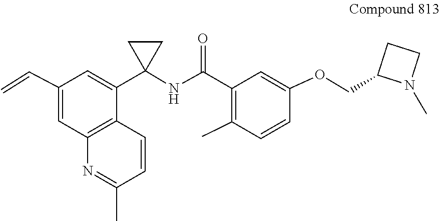 [SMILES: C=Cc1cc(C2(NC(=O)c3cc(OC[C@@H]4CCN4C)ccc3C)CC2)c2ccc(C)nc2c1]

Compound 813 was synthesized according to an analogous procedure to the one described for compound 653. M+H⁺=442.3 (LCMS); ¹H NMR (400 MHZ, DMSO-d₆) δ 10.92 (br d, J=3.4 Hz, 1H), 9.63-9.23 (m, 2H), 8.35-7.76 (m, 3H), 7.15-7.00 (m, 2H), 6.92 (dd, J=2.7, 8.4 Hz, 1H), 6.83-6.73 (m, 1H), 6.20 (d, J=17.6 Hz, 1H), 5.65 (br d, J=11.0 Hz, 1H), 4.68-4.58 (m, 1H), 4.40 (dd, J=8.2, 11.2 Hz, 1H), 4.22 (dd, J=3.1, 11.3 Hz, 1H), 4.05-3.93 (m, 1H), 3.91-3.77 (m, 1H), 2.92 (s, 3H), 2.83-2.66 (m, 3H), 2.40-2.26 (m, 2H), 1.98 (s, 3H), 1.42 (br s, 2H), 1.33 (br s, 2H).

### Example 587: (S)-2-Methyl-5-((1-methylpyrrolidin-2-yl)methoxy)-N-(1-(7-(prop-1-en-2-yl)quinolin-5-yl)cyclopropyl)benzamide (Compound 719)

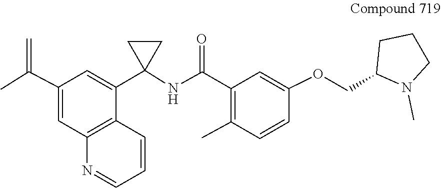 [SMILES: C=C(C)c1cc(C2(NC(=O)c3cc(OC[C@@H]4CCCN4C)ccc3C)CC2)c2cccnc2c1]

Compound 719 was synthesized according to an analogous procedure to the one described for compound 653. M+H⁺=456.1 (LCMS); ¹H NMR (400 MHZ, DMSO-d₆) δ 9.44-9.33 (m, 1H), 9.27-9.19 (m, 1H), 9.13-9.03 (m, 1H), 8.26-8.19 (m, 1H), 8.09-8.04 (m, 1H), 7.88-7.78 (m, 1H), 7.12-7.05 (m, 1H), 6.98-6.88 (m, 1H), 6.77-6.67 (m, 1H), 5.86-5.73 (m, 1H), 5.52-5.28 (m, 1H), 4.27-4.10 (m, 2H), 3.84-3.69 (m, 1H), 3.16-3.03 (m, 1H), 2.92-2.84 (m, 3H), 2.29-2.16 (m, 4H), 2.10-1.69 (m, 7H), 1.44-1.38 (m, 2H), 1.34-1.27 (m, 2H).

### Example 588: (S,E)-N-(1-(7-(2-Cyclopropylvinyl)quinolin-5-yl)cyclopropyl)-2-methyl-5-((1-methylazetidin-2-yl)methoxy)benzamide (Compound 684)

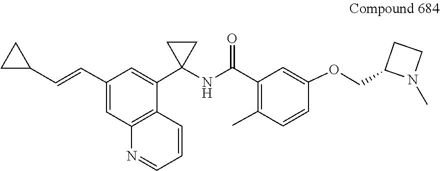 [SMILES: Cc1ccc(OC[C@@H]2CCN2C)cc1C(=O)NC1(c2cc(/C=C/C3CC3)cc3ncccc23)CC1]

Compound 684 was synthesized according to an analogous procedure to the one described for compound 653. M+H⁺=468.3 (LCMS); ¹H NMR (400 MHZ, DMSO-d₆) δ 10.04-9.84 (m, 1H), 9.22-9.09 (m, 2H), 8.99-8.90 (m, 1H), 8.00 (s, 1H), 7.80 (s, 1H), 7.63 (dd, J=4.4, 8.5 Hz, 1H), 7.11 (d, J=8.6 Hz, 1H), 7.00-6.87 (m, 1H), 6.80-6.67 (m, 2H), 6.17 (dd, J=9.4, 15.8 Hz, 1H), 4.65-4.58 (m, 1H), 4.23 (br d, J=5.7 Hz, 2H), 4.03-3.99 (m, 1H), 3.89-3.84 (m, 1H), 2.94-2.76 (m, 3H), 2.40-2.31 (m, 2H), 1.97 (s, 3H), 1.76-1.64 (m, 1H), 1.41-1.22 (m, 3H), 0.93-0.84 (m, 2H), 0.69-0.62 (m, 2H).

### Example 589: (S,E)-2-Methyl-5-((1-methylazetidin-2-yl)methoxy)-N-(1-(7-(3-methylstyryl)quinolin-5-yl)cyclopropyl)benzamide (Compound 690)

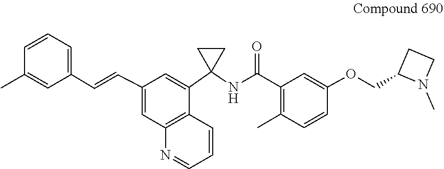

Compound 690 was synthesized according to an analogous procedure to the one described for compound 653. M+H⁺=518.3 (LCMS); ¹H NMR (400 MHZ, DMSO-d₆) § 10.59-10.46 (m, 1H), 9.43-9.34 (m, 1H), 9.27 (s, 1H), 9.08 (br d, J=3.9 Hz, 1H), 8.33 (s, 1H), 8.15 (s, 1H), 7.81 (br dd, J=3.3, 7.5 Hz, 1H), 7.61 (s, 1H), 7.57 (s, 2H), 7.57-7.53 (m, 1H), 7.33 (t, J=7.6 Hz, 1H), 7.17 (d, J=7.7 Hz, 1H), 7.10 (d, J=8.4 Hz, 1H), 6.92 (dd, J=2.5, 8.3 Hz, 1H), 6.74 (d, J=2.6 Hz, 1H), 4.66-4.58 (m, 1H), 4.33 (dd, J=7.8, 11.3 Hz, 1H), 4.25-4.19 (m, 1H), 4.03-3.98 (m, 1H), 3.88-3.84 (m, 1H), 2.81 (d, J=5.1 Hz, 3H), 2.41-2.25 (m, 5H), 1.98 (s, 3H), 1.43 (br s, 2H), 1.40-1.35 (m, 2H).

### Example 590: (S)-2-Methyl-5-((1-methylazetidin-2-yl)methoxy)-N-(1-(7-(prop-1-yn-1-yl)quinolin-5-yl)cyclopropyl)benzamide (Compound 721)

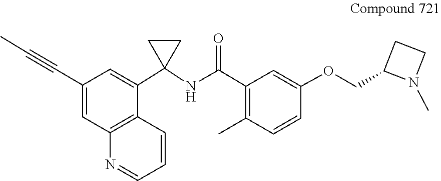 [SMILES: CC#Cc1cc(C2(NC(=O)c3cc(OC[C@@H]4CCN4C)ccc3C)CC2)c2cccnc2c1]

Compound 721 was synthesized according to an analogous procedure to the one described for compound 653. M+H⁺=440.1 (LCMS); ¹H NMR (400 MHZ, DMSO-d₆) δ 10.10-9.89 (m, 1H), 9.16 (s, 1H), 9.09 (br d, J=8.4 Hz, 1H), 8.96 (br d, J=3.4 Hz, 1H), 7.95 (s, 1H), 7.84 (s, 1H), 7.65 (dd, J=4.3, 8.5 Hz, 1H), 7.09 (d, J=8.4 Hz, 1H), 6.91 (dd, J=2.3, 8.4 Hz, 1H), 6.77-6.67 (m, 1H), 4.60 (br d, J=5.3 Hz, 1H), 4.22 (br d, J=5.1 Hz, 2H), 4.08-3.97 (m, 1H), 3.86 (br dd, J=5.6, 9.1 Hz, 1H), 2.88-2.68 (m, 3H), 2.43-2.25 (m, 2H), 2.14 (s, 3H), 1.94 (s, 3H), 1.35 (br s, 2H), 1.24 (br s, 2H).

### Example 591: (S)—N-(1-(7-(5-Acetylthiophen-2-yl)quinolin-5-yl)cyclopropyl)-2-methyl-5-((1-methylazetidin-2-yl)methoxy)benzamide (Compound 657)

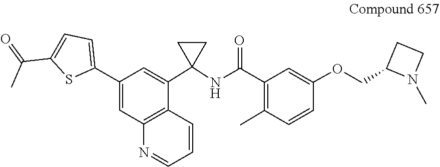 [SMILES: CC(=O)c1ccc(-c2cc(C3(NC(=O)c4cc(OC[C@@H]5CCN5C)ccc4C)CC3)c3cccnc3c2)s1]

Compound 657 was synthesized according to an analogous procedure to the one described for compound 653. M+H⁺=526.1 (LCMS); ¹H NMR (400 MHZ, DMSO-d₆) δ 9.91 (br s, 1H), 9.23-9.16 (m, 1H), 9.09 (d, J=8.6 Hz, 1H), 8.98 (dd, J=1.4, 4.1 Hz, 1H), 8.32 (d, J=1.5 Hz, 1H), 8.22 (d, J=1.9 Hz, 1H), 8.05 (d, J=4.0 Hz, 1H), 7.93 (d, J=4.0 Hz, 1H), 7.65 (dd, J=4.3, 8.5 Hz, 1H), 7.14-7.07 (m, 1H), 6.92 (dd, J=2.7, 8.4 Hz, 1H), 6.77-6.68 (m, 1H), 4.60 (br d, J=4.4 Hz, 1H), 4.25-4.20 (m, 2H), 3.87 (br dd, J=3.1, 9.4 Hz, 2H), 2.83 (d, J=4.9 Hz, 3H), 2.59 (s, 3H), 2.42-2.27 (m, 2H), 1.97 (s, 3H), 1.45-1.31 (m, 4H).

### Example 592: (S)—N-(1-(7-(5-Cyanothiophen-2-yl)quinolin-5-yl)cyclopropyl)-2-methyl-5-((1-methylazetidin-2-yl)methoxy)benzamide (Compound 754)

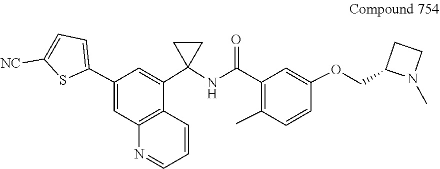 [SMILES: Cc1ccc(OC[C@@H]2CCN2C)cc1C(=O)NC1(c2cc(-c3ccc(C#N)s3)cc3ncccc23)CC1]

Compound 754 was synthesized according to an analogous procedure to the one described for compound 653. M+H⁺=509.4 (LCMS); ¹H NMR (400 MHZ, DMSO-d₆) δ 10.67 (br d, J=4.0 Hz, 1H), 9.33-9.23 (m, 2H), 9.08 (d, J=3.3 Hz, 1H), 8.39 (s, 1H), 8.24 (d, J=1.8 Hz, 1H), 8.11 (d, J=4.0 Hz, 1H), 8.01 (d, J=4.0 Hz, 1H), 7.79 (dd, J=4.4, 8.5 Hz, 1H), 7.09 (d, J=8.5 Hz, 1H), 6.91 (dd, J=2.8, 8.4 Hz, 1H), 6.73 (d, J=2.6 Hz, 1H), 4.69-4.56 (m, 1H), 4.42-4.30 (m, 1H), 4.21 (dd, J=3.2, 11.3 Hz, 1H), 4.04-3.95 (m, 1H), 3.89-3.80 (m, 1H), 2.80 (d, J=5.0 Hz, 3H), 2.38-2.26 (m, 2H), 1.96 (s, 3H), 1.45-1.40 (m, 2H), 1.37 (br s, 2H).

### Example 593: (S)-2-Methyl-5-((1-methylazetidin-2-yl)methoxy)-N-(1-(7-(2-methylthiazol-5-yl)quinolin-5-yl)cyclopropyl)benzamide (Compound 764)

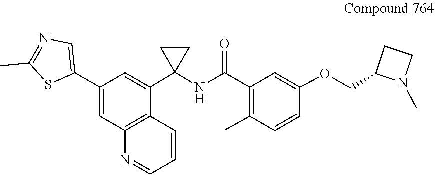

Compound 764 was synthesized according to an analogous procedure to the one described for compound 653. M+H⁺=499.2 (LCMS); ¹H NMR (400 MHZ, DMSO-d₆) δ 1.31-1.44 (m, 4H), 1.91-1.97 (m, 3H), 2.27-2.40 (m, 2H), 2.72-2.76 (m, 3H), 2.79-2.84 (m, 3H), 3.81-3.89 (m, 1H), 3.97-4.06 (m, 1H), 4.17-4.24 (m, 2H), 4.53-4.67 (m, 1H), 6.68-6.73 (m, 1H), 6.89-6.94 (m, 1H), 7.05-7.13 (m, 1H), 7.72-7.79 (m, 1H), 8.13 (s, 1H), 8.20 (s, 1H), 8.33 (s, 1H), 8.95-9.08 (m, 1H), 9.25 (br d, J=9.4 Hz, 1H).

### Example 594: (S)-2-Methyl-N-(1-(7-(5-methyl-1,3,4-thiadiazol-2-yl)quinolin-5-yl)cyclopropyl)-5-((1-methylazetidin-2-yl)methoxy)benzamide (Compound 691)

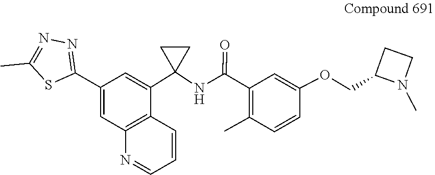

Compound 691 was synthesized according to an analogous procedure to the one described for compound 653. M+H⁺=500.1 (LCMS); ¹H NMR (400 MHZ, DMSO-d₆) δ 10.03-9.83 (m, 1H), 9.22 (s, 1H), 9.14 (d, J=8.5 Hz, 1H), 9.02 (dd, J=1.4, 4.1 Hz, 1H), 8.48 (d, J=1.8 Hz, 1H), 8.41 (d, J=1.3 Hz, 1H), 7.71 (dd, J=4.2, 8.6 Hz, 1H), 7.10 (d, J=8.6 Hz, 1H), 6.92 (dd, J=2.8, 8.4 Hz, 1H), 6.73 (d, J=2.8 Hz, 1H), 4.60 (br d, J=4.0 Hz, 1H), 4.27-4.17 (m, 2H), 4.08-3.99 (m, 1H), 3.93-3.82 (m, 1H), 2.86-2.80 (m, 6H), 2.41-2.28 (m, 2H), 1.96 (s, 3H), 1.43 (br s, 2H), 1.31 (br s, 2H).

### Example 595: (S)-2-Methyl-5-((1-methylazetidin-2-yl)methoxy)-N-(1-(7-(5-methylfuran-2-yl)quinolin-5-yl)cyclopropyl)benzamide (Compound 654)

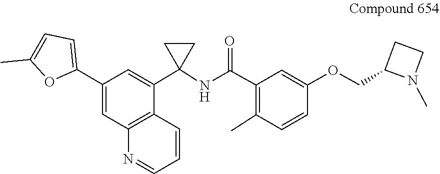

Compound 654 was synthesized according to an analogous procedure to the one described for compound 653. M+H⁺=482.2 (LCMS); ¹H NMR (400 MHZ, DMSO-d₆) δ 10.63-10.46 (m, 1H), 9.39 (br d, J=4.3 Hz, 1H), 9.28 (s, 1H), 9.09 (br d, J=4.8 Hz, 1H), 8.25 (d, J=11.5 Hz, 2H), 7.92-7.72 (m, 1H), 7.27 (d, J=2.8 Hz, 1H), 7.17-7.02 (m, 1H), 6.92 (dd, J=2.5, 8.4 Hz, 1H), 6.74 (d, J=2.6 Hz, 1H), 6.38 (d, J=3.0 Hz, 1H), 4.68-4.58 (m, 1H), 4.37-4.28 (m, 1H), 4.26-4.18 (m, 1H), 3.99 (br dd, J=4.6, 9.0 Hz, 1H), 3.88-3.81 (m, 1H), 2.81 (d, J=5.0 Hz, 3H), 2.44 (s, 3H), 2.37-2.29 (m, 2H), 1.97 (s, 3H), 1.42 (br s, 2H), 1.35 (br s, 2H).

### Example 596: (S)—N-(1-(7-(Furan-3-yl)quinolin-5-yl)cyclopropyl)-2-methyl-5-((1-methylazetidin-2-yl)methoxy)benzamide (Compound 655)

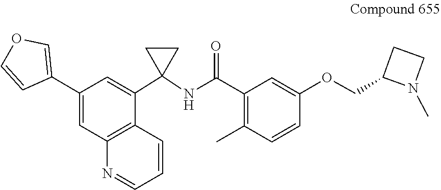 [SMILES: Cc1ccc(OC[C@@H]2CCN2C)cc1C(=O)NC1(c2cc(-c3ccoc3)cc3ncccc23)CC1]

Compound 655 was synthesized according to an analogous procedure to the one described for compound 653. M+H⁺=468.2 (LCMS); ¹H NMR (400 MHZ, DMSO-d₆) δ 9.70 (br d, J=8.4 Hz, 1H), 9.36 (s, 1H), 9.22 (d, J=5.1 Hz, 1H), 8.62 (s, 1H), 8.36 (s, 1H), 8.29 (s, 1H), 8.05 (dd, J=5.3, 8.5 Hz, 1H), 7.91 (s, 1H), 7.17 (s, 1H), 7.14-7.03 (m, 1H), 6.92 (dd, J=2.6, 8.3 Hz, 1H), 6.75 (d, J=2.6 Hz, 1H), 4.62 (br dd, J=2.9, 7.4 Hz, 1H), 4.40-4.26 (m, 1H), 4.25-4.14 (m, 1H), 4.01 (dt, J=4.4, 9.6 Hz, 1H), 3.85 (q, J=9.7 Hz, 1H), 2.81 (s, 3H), 2.42-2.23 (m, 2H), 1.96 (s, 3H), 1.45 (br s, 2H), 1.42 (br s, 2H).

### Example 597: (S)—N-(1-(7-(2,5-Dimethylfuran-3-yl)quinolin-5-yl)cyclopropyl)-2-methyl-5-((1-methylazetidin-2-yl)methoxy)benzamide (Compound 671)

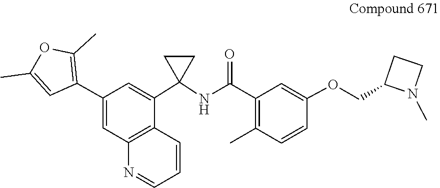

Compound 671 was synthesized according to an analogous procedure to the one described for compound 653. M+H⁺=496.1 (LCMS); ¹H NMR (400 MHZ, CD₃OD) δ 9.60 (d, J=8.5 Hz, 1H), 9.07 (dd, J=1.3, 5.1 Hz, 1H), 8.37 (d, J=1.5 Hz, 1H), 8.06 (s, 1H), 7.91 (dd, J=5.1, 8.5 Hz, 1H), 7.13 (d, J=8.4 Hz, 1H), 6.97 (dd, J=2.6, 8.3 Hz, 1H), 6.80 (d, J=2.6 Hz, 1H), 6.47 (s, 1H), 4.74-4.61 (m, 1H), 4.31-4.26 (m, 1H), 4.24-4.14 (m, 2H), 4.00-3.90 (m, 1H), 2.95 (s, 3H), 2.61 (s, 3H), 2.57-2.51 (m, 2H), 2.34 (s, 3H), 2.03 (s, 4H), 1.60-1.53 (m, 2H), 1.48-1.41 (m, 2H).

### Example 598: (S)-2-Methyl-5-((1-methylazetidin-2-yl)methoxy)-N-(1-(7-(2-methyloxazol-5-yl)quinolin-5-yl)cyclopropyl)benzamide (Compound 716)

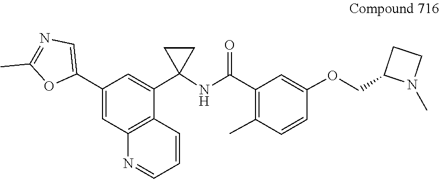

Compound 716 was synthesized according to an analogous procedure to the one described for compound 653. M+H⁺=483.4 (LCMS); ¹H NMR (400 MHZ, DMSO-d₆) δ 11.19-11.07 (m, 1H), 9.83-9.63 (m, 1H), 9.47-9.39 (m, 1H), 9.24 (d, J=4.3 Hz, 1H), 8.57-8.28 (m, 2H), 8.13-7.96 (m, 2H), 7.21-7.03 (m, 1H), 6.91 (dd, J=2.6, 8.3 Hz, 1H), 6.77 (d, J=2.6 Hz, 1H), 4.70-4.60 (m, 1H), 4.43 (br dd, J=8.3, 11.2 Hz, 1H), 4.23-4.19 (m, 1H), 3.83 (br d, J=6.9 Hz, 2H), 2.84-2.65 (m, 3H), 2.59 (s, 3H), 2.39-2.25 (m, 1H), 2.42-2.17 (m, 2H), 2.10-1.90 (m, 3H), 1.60-1.24 (m, 4H).

### Example 599: (S)-2-Methyl-N-(1-(7-(5-methyl-1,3,4-oxadiazol-2-yl)quinolin-5-yl)cyclopropyl)-5-((1-methylazetidin-2-yl)methoxy)benzamide (Compound 693)

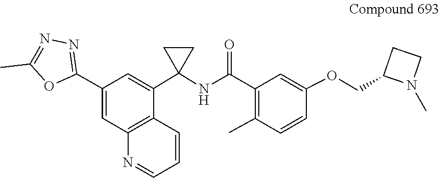

Compound 693 was synthesized according to an analogous procedure to the one described for compound 653. M+H⁺=484.3 (LCMS); ¹H NMR (400 MHZ, DMSO-d₆) δ 9.95-9.82 (m, 1H), 9.27-9.18 (m, 1H), 9.16 (s, 1H), 9.05 (dd, J=1.5, 4.1 Hz, 1H), 8.48 (s, 1H), 8.44 (d, J=1.8 Hz, 1H), 7.74 (dd, J=4.1, 8.6 Hz, 1H), 7.19-7.07 (m, 1H), 6.92 (dd, J=2.8, 8.4 Hz, 1H), 6.81-6.70 (m, 1H), 4.66-4.53 (m, 1H), 4.42-4.36 (m, 1H), 4.27-4.18 (m, 2H), 4.07-3.97 (m, 1H), 3.86 (br dd, J=6.3, 9.5 Hz, 1H), 2.84 (d, J=4.9 Hz, 3H), 2.66 (s, 3H), 2.42-2.26 (m, 2H), 1.95 (s, 3H), 1.43 (br s, 2H), 1.30 (br s, 2H), 1.22-1.25 (m, 1H).

### Example 600: (S)-2-Methyl-N-(1-(2-methyl-7-(1-methyl-1H-pyrazol-4-yl)quinolin-5-yl)cyclopropyl)-5-((1-methylazetidin-2-yl)methoxy)benzamide (Compound 820)

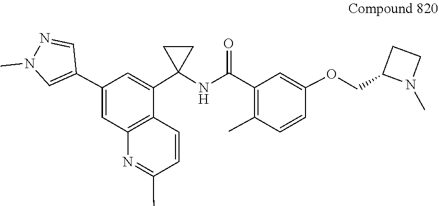

Compound 820 was synthesized according to an analogous procedure to the one described for compound 653. M+H⁺=496.3 (LCMS); ¹H NMR (400 MHZ, DMSO-d₆) δ 9.41 (d, J=8.8 Hz, 1H), 9.32-9.15 (m, 1H), 8.49 (s, 1H), 8.22 (d, J=0.9 Hz, 1H), 8.13 (d, J=4.6 Hz, 2H), 7.83 (d, J=8.8 Hz, 1H), 7.10 (d, J=8.5 Hz, 1H), 7.02-6.87 (m, 1H), 6.80-6.68 (m, 1H), 4.67-4.56 (m, 1H), 4.23 (d, J=5.3 Hz, 2H), 4.04 (dt, J=4.8, 9.5 Hz, 1H), 3.95 (s, 3H), 3.91-3.84 (m, 1H), 2.85 (d, J=10.6 Hz, 6H), 2.41-2.30 (m, 2H), 1.97 (s, 3H), 1.48-1.31 (m, 4H).

### Example 601: (S)—N-(1-(7-(Benzo[d]oxazol-2-yl)quinolin-5-yl)cyclopropyl)-2-methyl-5-((1-methylazetidin-2-yl)methoxy)benzamide (Compound 718)

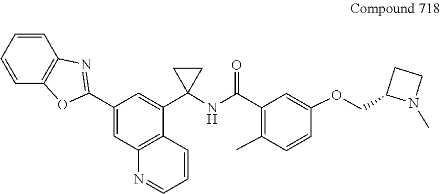 [SMILES: Cc1ccc(OC[C@@H]2CCN2C)cc1C(=O)NC1(c2cc(-c3nc4ccccc4o3)cc3ncccc23)CC1]

Compound 718 was synthesized according to an analogous procedure to the one described for compound 653. M+H⁺=519.3 (LCMS); ¹H NMR (400 MHZ, DMSO-d₆) δ 9.49-9.24 (m, 2H), 9.16 (d, J=3.4 Hz, 1H), 8.82 (s, 1H), 8.74 (s, 1H), 7.98-7.82 (m, 3H), 7.60-7.36 (m, 2H), 7.16-7.00 (m, 1H), 6.92 (dd, J=2.7, 8.3 Hz, 1H), 6.75 (d, J=2.6 Hz, 1H), 4.66-4.54 (m, 1H), 4.35-4.17 (m, 2H), 4.00 (td, J=4.8, 9.5 Hz, 1H), 3.85 (q, J=9.5 Hz, 1H), 2.86-2.68 (m, 3H), 2.42-2.21 (m, 2H), 1.97 (s, 3H), 1.48 (br s, 2H), 1.35 (br s, 2H).

### Example 602: (S)—N-(1-(7-(Imidazo[1,5-a]pyridin-1-yl)quinolin-5-yl)cyclopropyl)-2-methyl-5-((1-methylazetidin-2-yl)methoxy)benzamide (Compound 736)

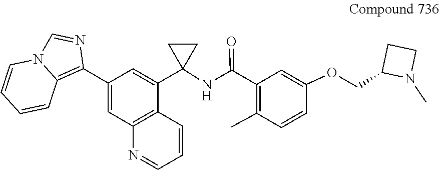 [SMILES: Cc1ccc(OC[C@@H]2CCN2C)cc1C(=O)NC1(c2cc(-c3ncn4ccccc34)cc3ncccc23)CC1]

Compound 736 was synthesized according to an analogous procedure to the one described for compound 653. M+H⁺=518.3 (LCMS); ¹H NMR (400 MHZ, DMSO-d₆) δ 9.93-9.80 (m, 1H), 9.34-9.26 (m, 1H), 9.26-9.21 (m, 1H), 9.05 (br d, J=4.5 Hz, 1H), 8.66 (s, 2H), 8.52 (d, J=7.0 Hz, 1H), 8.43 (s, 1H), 8.15 (d, J=9.0 Hz, 1H), 7.78-7.69 (m, 1H), 7.19-7.07 (m, 2H), 6.97-6.84 (m, 2H), 6.76 (d, J=2.8 Hz, 1H), 4.61 (br d, J=4.3 Hz, 1H), 4.24-4.21 (m, 2H), 4.05-4.01 (m, 1H), 3.87 (br dd, J=6.4, 9.4 Hz, 1H), 2.84 (d, J=4.9 Hz, 3H), 2.38-2.28 (m, 2H), 1.99 (s, 3H), 1.45 (br s, 2H), 1.34 (br s, 2H).

### Example 603: (S)—N-(1-(7-(Cyclohex-1-en-1-yl)quinolin-5-yl)cyclopropyl)-2-methyl-5-((1-methylazetidin-2-yl)methoxy)benzamide (Compound 663)

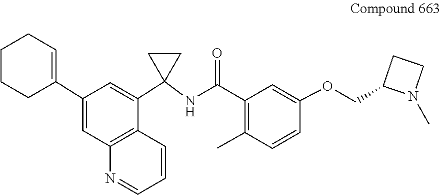 [SMILES: Cc1ccc(OC[C@@H]2CCN2C)cc1C(=O)NC1(c2cc(C3=CCCCC3)cc3ncccc23)CC1]

Compound 663 was synthesized according to an analogous procedure to the one described for compound 653. M+H⁺=482.3 (LCMS); ¹H NMR (400 MHZ, DMSO-d₆) δ 10.77-10.37 (m, 1H), 9.53-9.38 (m, 1H), 9.25 (br s, 1H), 9.16-9.04 (m, 1H), 8.20 (br s, 1H), 8.03 (br s, 1H), 7.92-7.80 (m, 1H), 7.09 (d, J=8.4 Hz, 1H), 6.92 (dd, J=2.6, 8.4 Hz, 1H), 6.73 (d, J=2.4 Hz, 1H), 6.63 (br s, 1H), 4.66-4.55 (m, 1H), 4.38-4.28 (m, 1H), 4.21 (dd, J=3.1, 11.2 Hz, 1H), 4.03-3.94 (m, 1H), 3.91-3.75 (m, 2H), 2.81 (d, J=4.9 Hz, 3H), 2.70-2.65 (m, 1H), 2.37-2.28 (m, 4H), 1.96 (s, 3H), 1.86-1.78 (m, 2H), 1.72-1.64 (m, 2H), 1.41 (br s, 2H), 1.31 (br s, 2H).

### Example 604: (S)—N-(1-(7-(4-Fluorophenyl)quinolin-5-yl)cyclopropyl)-2-methyl-5-((1-methylazetidin-2-yl)methoxy)benzamide (Compound 678)

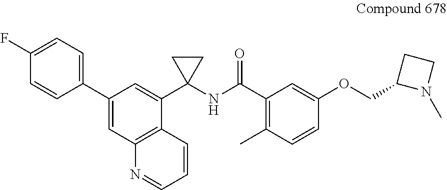 [SMILES: Cc1ccc(OC[C@@H]2CCN2C)cc1C(=O)NC1(c2cc(-c3ccc(F)cc3)cc3ncccc23)CC1]

Compound 678 was synthesized according to an analogous procedure to the one described for compound 653. M+H⁺=496.1 (LCMS); ¹H NMR (400 MHz, DMSO-d₆) δ 9.94 (br s, 1H), 9.23-9.13 (m, 2H), 9.01 (dd, J=1.5, 4.4 Hz, 1H), 8.21 (s, 2H), 7.99-7.89 (m, 2H), 7.68 (dd, J=4.3, 8.6 Hz, 1H), 7.46-7.36 (m, 2H), 7.13-7.07 (m, 1H), 6.97-6.88 (m, 1H), 6.77-6.70 (m, 1H), 4.60 (br d, J=4.6 Hz, 1H), 4.22 (d, J=5.5 Hz, 2H), 4.01 (dt, J=5.0, 9.6 Hz, 1H), 3.91-3.85 (m, 1H), 2.87-2.69 (m, 3H), 2.41-2.29 (m, 2H), 1.97 (s, 3H), 1.39 (br d, J=14.3 Hz, 4H).

### Example 605: (S)—N-(1-(7-(3-Chloro-4-fluorophenyl)quinolin-5-yl)cyclopropyl)-2-methyl-5-((1-methylazetidin-2-yl)methoxy)benzamide (Compound 687)

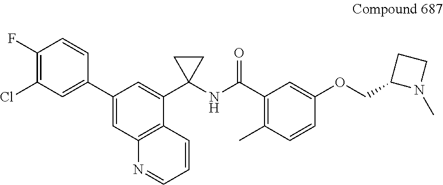 [SMILES: Cc1ccc(OC[C@@H]2CCN2C)cc1C(=O)NC1(c2cc(-c3ccc(F)c(Cl)c3)cc3ncccc23)CC1]

Compound 687 was synthesized according to an analogous procedure to the one described for compound 653. M+H⁺=530.3/532.3 (LCMS); ¹H NMR (400 MHZ, DMSO-d₆) δ 9.88-9.80 (m, 1H), 9.20-9.16 (m, 1H), 9.11 (d, J=8.9 Hz, 1H), 8.98 (dd, J=1.5, 4.1 Hz, 1H), 8.25 (d, J=1.4 Hz, 1H), 8.18 (d, J=1.8 Hz, 1H), 8.13 (dd, J=2.3, 7.1 Hz, 1H), 7.95-7.88 (m, 1H), 7.68-7.57 (m, 2H), 7.10 (d, J=8.4 Hz, 1H), 6.91 (dd, J=2.7, 8.3 Hz, 1H), 6.71 (d, J=2.6 Hz, 1H), 4.65-4.53 (m, 1H), 4.25-4.17 (m, 2H), 4.07-3.94 (m, 1H), 3.90-3.77 (m, 1H), 2.83 (d, J=4.9 Hz, 3H), 2.29 (s, 2H), 1.97 (s, 3H), 1.39 (s, 4H).

### Example 606: (S)-2-Methyl-5-((1-methylazetidin-2-yl)methoxy)-N-(1-(7-(6-oxo-1,6-dihydropyridin-3-yl)quinolin-5-yl)cyclopropyl)benzamide (Compound 672)

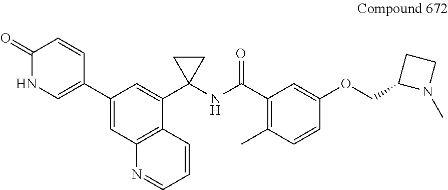 [SMILES: Cc1ccc(OC[C@@H]2CCN2C)cc1C(=O)NC1(c2cc(-c3ccc(=O)[nH]c3)cc3ncccc23)CC1]

Compound 672 was synthesized according to an analogous procedure to the one described for compound 653. M+H⁺=495.2 (LCMS); ¹H NMR (400 MHZ, DMSO-d₆) δ 9.95-9.77 (m, 1H), 9.24-9.05 (m, 2H), 9.01-8.90 (m, 1H), 8.19-7.93 (m, 4H), 7.62 (dd, J=4.3, 8.8 Hz, 1H), 7.19-7.04 (m, 1H), 7.00-6.87 (m, 1H), 6.77-6.65 (m, 1H), 6.53 (d, J=9.7 Hz, 1H), 4.66-4.52 (m, 1H), 4.25-4.15 (m, 2H), 4.08-3.99 (m, 1H), 3.86 (br dd, J=6.3, 9.7 Hz, 1H), 2.83 (d, J=5.0 Hz, 3H), 2.44-2.30 (m, 2H), 1.97 (s, 3H), 1.37 (br s, 4H).

### Example 607: (S)-2-Methyl-5-((1-methylazetidin-2-yl)methoxy)-N-(1-(7-(pyrimidin-2-yl)quinolin-5-yl)cyclopropyl)benzamide (Compound 681)

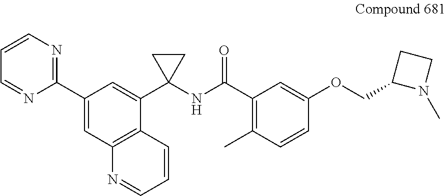 [SMILES: Cc1ccc(OC[C@@H]2CCN2C)cc1C(=O)NC1(c2cc(-c3ncccn3)cc3ncccc23)CC1]

Compound 681 was synthesized according to an analogous procedure to the one described for compound 653. M+H⁺=480.2 (LCMS); ¹H NMR (400 MHZ, DMSO-d₆) δ 9.97-9.82 (m, 1H), 9.26-9.13 (m, 2H), 9.07-9.00 (m, 3H), 8.99-8.92 (m, 2H), 7.70 (dd, J=4.1, 8.5 Hz, 1H), 7.56 (t, J=4.9 Hz, 1H), 7.15-7.06 (m, 1H), 6.99-6.87 (m, 1H), 6.79-6.68 (m, 1H), 4.61 (br dd, J=3.3, 6.1 Hz, 1H), 4.29-4.16 (m, 2H), 4.08-3.96 (m, 1H), 3.86 (br dd, J=6.1, 9.5 Hz, 1H), 2.88-2.65 (m, 3H), 2.42-2.26 (m, 2H), 1.97 (s, 3H), 1.44 (br s, 2H), 1.29 (br s, 2H).

### Example 608: (S)-2-Methyl-5-((1-methylazetidin-2-yl)methoxy)-N-(1-(7-(2-(piperidin-1-yl)pyrimidin-5-yl)quinolin-5-yl)cyclopropyl)benzamide (Compound 701)

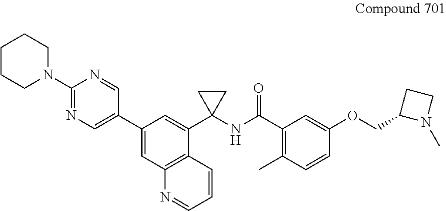 [SMILES: Cc1ccc(OC[C@@H]2CCN2C)cc1C(=O)NC1(c2cc(-c3cnc(N4CCCCC4)nc3)cc3ncccc23)CC1]

Compound 701 was synthesized according to an analogous procedure to the one described for compound 653. M+H⁺=563.3 (LCMS); ¹H NMR (400 MHZ, DMSO-d₆) δ 11.32-11.16 (m, 1H), 9.89-9.76 (m, 1H), 9.52-9.44 (m, 1H), 9.30 (d, J=4.4 Hz, 1H), 8.94 (s, 2H), 8.50 (s, 1H), 8.41 (d, J=1.5 Hz, 1H), 8.11 (dd, J=5.3, 8.6 Hz, 1H), 7.08 (d, J=8.6 Hz, 1H), 6.91 (dd, J=2.7, 8.4 Hz, 1H), 6.78 (d, J=2.6 Hz, 1H), 4.69-4.59 (m, 1H), 4.45 (dd, J=8.4, 11.3 Hz, 1H), 4.21 (dd, J=3.0, 11.1 Hz, 1H), 4.03-3.92 (m, 1H), 3.90-3.86 (m, 4H), 3.85-3.78 (m, 1H), 2.79 (d, J=5.0 Hz, 3H), 2.41-2.21 (m, 2H), 2.01-1.97 (m, 3H), 1.71-1.64 (m, 2H), 1.62-1.53 (m, 4H), 1.53-1.41 (m, 4H).

### Example 609: (S)-2-Methyl-5-((1-methylazetidin-2-yl)methoxy)-N-(1-(7-(5-morpholinopyridin-3-yl)quinolin-5-yl)cyclopropyl)benzamide (Compound 699)

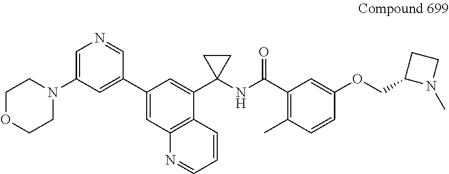 [SMILES: Cc1ccc(OC[C@@H]2CCN2C)cc1C(=O)NC1(c2cc(-c3cncc(N4CCOCC4)c3)cc3ncccc23)CC1]

Compound 699 was synthesized according to an analogous procedure to the one described for compound 653. M+H⁺=564.4 (LCMS); ¹H NMR (400 MHZ, DMSO-d₆) δ 10.09-9.89 (m, 1H), 9.28-9.19 (m, 1H), 9.15 (d, J=8.5 Hz, 1H), 9.02 (dd, J=1.4, 4.1 Hz, 1H), 8.66 (s, 1H), 8.48 (br d, J=2.0 Hz, 1H), 8.43 (s, 1H), 8.22 (d, J=1.7 Hz, 1H), 8.14-8.06 (m, 1H), 7.69 (dd, J=4.2, 8.6 Hz, 1H), 7.14-7.06 (m, 1H), 6.96-6.88 (m, 1H), 6.72 (d, J=2.6 Hz, 1H), 4.65-4.57 (m, 1H), 4.25-4.19 (m, 2H), 4.07-3.99 (m, 1H), 3.93-3.85 (m, 1H), 3.84-3.79 (m, 4H), 3.51-3.40 (m, 4H), 2.85 (br d, J=4.4 Hz, 3H), 2.39-2.27 (m, 2H), 1.96 (s, 3H), 1.42 (br s, 4H).

### Example 610: (S)—N-(1-(7-(Imidazo[1,2-a]pyridin-6-yl)quinolin-5-yl)cyclopropyl)-2-methyl-5-((1-methylazetidin-2-yl)methoxy)benzamide (Compound 702)

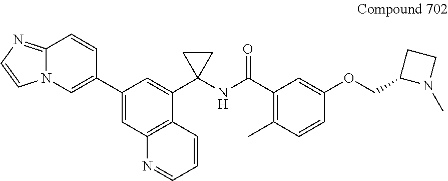 [SMILES: Cc1ccc(OC[C@@H]2CCN2C)cc1C(=O)NC1(c2cc(-c3ccc4nccn4c3)cc3ncccc23)CC1]

Compound 702 was synthesized according to an analogous procedure to the one described for compound 653. M+H⁺=518.2 (LCMS); ¹H NMR (400 MHZ, DMSO-d₆) δ 10.84-10.70 (m, 1H), 9.59 (s, 1H), 9.37-9.33 (m, 1H), 9.26 (br d, J=8.8 Hz, 1H), 9.11-9.07 (m, 1H), 8.56-8.51 (m, 1H), 8.44 (dd, J=1.4, 9.5 Hz, 2H), 8.33-8.27 (m, 2H), 8.15 (d, J=9.2 Hz, 1H), 7.83-7.74 (m, 1H), 7.09 (d, J=8.3 Hz, 1H), 6.92 (dd, J=2.6, 8.3 Hz, 1H), 6.73 (d, J=2.9 Hz, 1H), 4.69-4.57 (m, 1H), 4.37 (dd, J=8.1, 11.4 Hz, 1H), 4.21 (dd, J=3.2, 11.3 Hz, 1H), 4.03-3.95 (m, 1H), 3.81 (s, 1H), 2.80 (d, J=5.0 Hz, 3H), 2.34-2.27 (m, 2H), 1.96 (s, 3H), 1.45-1.40 (m, 4H).

### Example 611: (S)—N-(1-(7-([1,2,4]Triazolo[1,5-a]pyridin-6-yl)quinolin-5-yl)cyclopropyl)-2-methyl-5-((1-methylazetidin-2-yl)methoxy)benzamide (Compound 700)

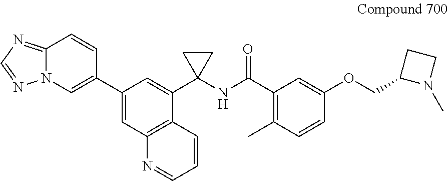 [SMILES: Cc1ccc(OC[C@@H]2CCN2C)cc1C(=O)NC1(c2cc(-c3ccc4ncnn4c3)cc3ncccc23)CC1]

Compound 700 was synthesized according to an analogous procedure to the one described for compound 653. M+H⁺=519.2 (LCMS); ¹H NMR (400 MHZ, DMSO-d₆) δ 11.19-10.91 (m, 1H), 9.85-9.63 (m, 2H), 9.54-9.39 (m, 1H), 9.35-9.24 (m, 1H), 8.68 (s, 1H), 8.62 (s, 1H), 8.50 (s, 1H), 8.26-8.21 (m, 1H), 8.14-8.06 (m, 2H), 7.12-7.07 (m, 1H), 6.94-6.89 (m, 1H), 6.77 (d, J=2.8 Hz, 1H), 4.65-4.61 (m, 1H), 4.42 (dd, J=8.3, 11.4 Hz, 1H), 4.21 (dd, J=3.2, 11.1 Hz, 1H), 4.02-3.93 (m, 1H), 3.89-3.76 (m, 1H), 2.79 (d, J=4.8 Hz, 3H), 2.39-2.21 (m, 2H), 2.01-1.96 (m, 3H), 1.57-1.50 (m, 2H), 1.47 (br s, 2H).

### Example 612: (S)-2-Methyl-5-(2-(methylamino)propoxy)-N-(1-(7-vinylquinolin-5-yl)cyclopropyl)benzamide (Compound 661)

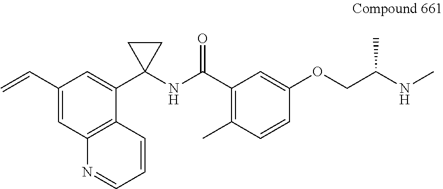 [SMILES: C=Cc1cc(C2(NC(=O)c3cc(OC[C@H](C)NC)ccc3C)CC2)c2cccnc2c1]

Compound 661 was synthesized according to an analogous procedure to the one described for compound 660. M+H⁺=416.2 (LCMS); ¹H NMR (400 MHZ, DMSO-d₆) δ 9.54 (br d, J=6.1 Hz, 1H), 9.32 (s, 1H), 9.23-9.05 (m, 2H), 9.03-8.89 (m, 1H), 8.23 (s, 1H), 8.15 (s, 1H), 7.96 (br d, J=5.5 Hz, 1H), 7.14-7.01 (m, 2H), 6.91 (dd, J=2.7, 8.3 Hz, 1H), 6.73 (d, J=2.6 Hz, 1H), 6.21 (d, J=17.6 Hz, 1H), 5.64 (d, J=10.9 Hz, 1H), 4.16-4.11 (m, 1H), 4.04 (br dd, J=6.2, 10.7 Hz, 1H), 3.51 (br d, J=4.1 Hz, 1H), 2.56-2.52 (m, 3H), 1.97 (s, 3H), 1.42 (br s, 2H), 1.34 (br s, 2H), 1.28 (d, J=6.8 Hz, 3H).

### Example 613: (S)-5-(2-Aminopropoxy)-2-methyl-N-(1-(7-vinylquinolin-5-yl)cyclopropyl)benzamide (Compound 735)

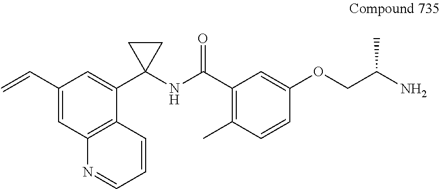 [SMILES: C=Cc1cc(C2(NC(=O)c3cc(OC[C@H](C)N)ccc3C)CC2)c2cccnc2c1]

Compound 735 was synthesized according to an analogous procedure to the one described for compound 660. M+H⁺=402.3 (LCMS); ¹H NMR (400 MHZ, DMSO-d₆) δ 9.57 (br d, J=8.4 Hz, 1H), 9.33 (s, 1H), 9.18 (d, J=4.0 Hz, 1H), 8.28-8.10 (m, 5H), 7.97 (dd, J=5.0, 8.5 Hz, 1H), 7.15-7.00 (m, 2H), 6.91 (dd, J=2.6, 8.4 Hz, 1H), 6.73 (d, J=2.6 Hz, 1H), 6.22 (d, J=17.6 Hz, 1H), 5.65 (d, J=10.9 Hz, 1H), 4.07-4.02 (m, 1H), 3.95-3.89 (m, 1H), 3.60-3.46 (m, 2H), 1.98 (s, 3H), 1.51-1.30 (m, 4H), 1.26 (d, J=6.7 Hz, 3H).

### Example 614: (S)-5-(2-Aminopropoxy)-2-methyl-N-(1-(7-(prop-1-en-2-yl)quinolin-5-yl)cyclopropyl)benzamide (Compound 734)

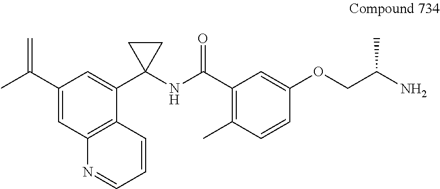 [SMILES: C=C(C)c1cc(C2(NC(=O)c3cc(OC[C@H](C)N)ccc3C)CC2)c2cccnc2c1]

Compound 734 was synthesized according to an analogous procedure to the one described for compound 660. M+H⁺=432.1 (LCMS); ¹H NMR (400 MHZ, DMSO-d₆) δ 9.59 (br d, J=8.4 Hz, 1H), 9.32 (s, 1H), 9.20 (d, J=4.1 Hz, 1H), 8.32-8.19 (m, 6H), 7.98 (dd, J=5.0, 8.5 Hz, 1H), 7.08 (d, J=8.5 Hz, 1H), 6.90 (dd, J=2.8, 8.4 Hz, 1H), 6.71 (d, J=2.6 Hz, 1H), 5.91-5.73 (m, 1H), 5.49 (s, 1H), 4.07-4.03 (m, 1H), 3.95-3.91 (m, 1H), 3.57-3.44 (m, 2H), 2.27 (s, 3H), 1.97 (s, 4H), 1.45-1.39 (m, 2H), 1.34 (br s, 2H), 1.25 (d, J=6.8 Hz, 4H).

### Example 615: N-(1-(7-(5-((((1S,3R)-3-Hydroxycyclopentyl)amino)methyl)thiophen-2-yl)quinolin-5-yl)cyclopropyl)-2-methyl-5-(((S)-1-methylazetidin-2-yl)methoxy)benzamide (Compound 751)

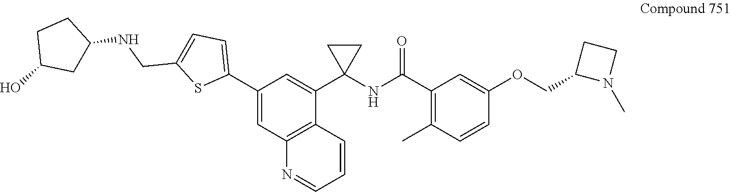 [SMILES: Cc1ccc(OC[C@@H]2CCN2C)cc1C(=O)NC1(c2cc(-c3ccc(CN[C@H]4CC[C@@H](O)C4)s3)cc3ncccc23)CC1]

Compound 751 was synthesized according to an analogous procedure to the one described for compound 669. M+H⁺=597.4 (LCMS); ¹H NMR (400 MHZ, DMSO-d₆) δ 10.49-10.38 (m, 1H), 9.26 (s, 1H), 9.21-9.12 (m, 3H), 9.00 (d, J=4.0 Hz, 1H), 8.25-8.17 (m, 2H), 7.78 (d, J=3.5 Hz, 1H), 7.69 (dd, J=4.3, 8.3 Hz, 1H), 7.43 (d, J=3.5 Hz, 1H), 7.09 (d, J=8.5 Hz, 1H), 6.97-6.87 (m, 1H), 6.76-6.68 (m, 1H), 4.67-4.56 (m, 1H), 4.44 (br t, J=5.0 Hz, 2H), 4.36-4.27 (m, 1H), 4.24-4.18 (m, 1H), 4.12 (quin, J=5.1 Hz, 1H), 4.05-3.94 (m, 1H), 3.91-3.80 (m, 1H), 2.81 (d, J=5.0 Hz, 3H), 2.73-2.64 (m, 1H), 2.36-2.27 (m, 2H), 2.23-2.18 (m, 1H), 2.25-2.16 (m, 1H), 2.26-2.16 (m, 1H), 1.74-1.63 (m, 1H), 1.77-1.61 (m, 3H), 1.43-1.39 (m, 1H), 1.41 (br s, 1H), 1.32 (br s, 2H).

### Example 616: (S)—N-(1-(7-(5-((Dimethylamino)methyl)thiophen-2-yl)quinolin-5-yl)cyclopropyl)-2-methyl-5-((1-methylazetidin-2-yl)methoxy)benzamide (Compound 756)

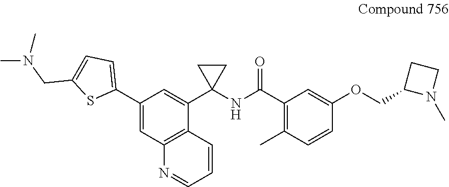

Compound 756 was synthesized according to an analogous procedure to the one described for compound 669. M+H⁺=541.4 (LCMS); ¹H NMR (400 MHZ, DMSO-d₆) δ 10.96-10.58 (m, 1H), 9.47-9.26 (m, 2H), 9.10 (br s, 1H), 8.45-8.18 (m, 2H), 7.99-7.75 (m, 2H), 7.50 (br s, 1H), 7.17-7.03 (m, 1H), 6.99-6.87 (m, 1H), 6.75 (s, 1H), 4.60 (br d, J=5.1 Hz, 2H), 4.42-4.34 (m, 1H), 4.21 (br dd, J=2.9, 11.3 Hz, 1H), 4.05-3.94 (m, 1H), 3.91-3.79 (m, 2H), 2.84-2.72 (m, 9H), 2.36-2.27 (m, 2H), 1.96 (s, 3H), 1.50-1.30 (m, 3H).

### Example 617: (S)—N-(1-(7-(5-((3,3-Difluoropyrrolidin-1-yl)methyl)thiophen-2-yl)quinolin-5-yl)cyclopropyl)-2-methyl-5-((1-methylazetidin-2-yl)methoxy)benzamide (Compound 805)

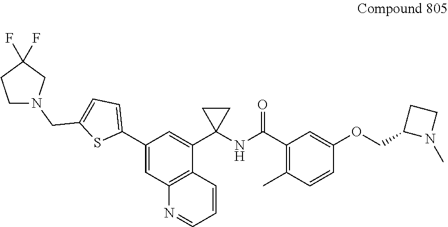 [SMILES: Cc1ccc(OC[C@@H]2CCN2C)cc1C(=O)NC1(c2cc(-c3ccc(CN4CCC(F)(F)C4)s3)cc3ncccc23)CC1]

Compound 805 was synthesized according to an analogous procedure to the one described for compound 669. M+H⁺=603.5 (LCMS); ¹H NMR (400 MHZ, DMSO-d₆) δ 9.14 (s, 1H), 9.03 (d, J=8.3 Hz, 1H), 8.93-8.88 (m, 1H), 8.11 (s, 2H), 7.63 (d, J=3.5 Hz, 1H), 7.55 (sxt, J=4.2 Hz, 1H), 7.09 (d, J=3.6 Hz, 1H), 7.03 (d, J=8.4 Hz, 1H), 6.83 (dd, J=2.6, 8.4 Hz, 1H), 6.61 (d, J=2.8 Hz, 1H), 3.90 (s, 2H), 3.86 (d, J=5.4 Hz, 2H), 3.26-3.16 (m, 2H), 2.98 (t, J=13.3 Hz, 2H), 2.80 (t, J=7.2 Hz, 2H), 2.75-2.64 (m, 2H), 2.34-2.27 (m, 2H), 2.20 (s, 3H), 1.95 (s, 3H), 1.90-1.79 (m, 1H), 1.42-1.36 (m, 2H), 1.32-1.26 (m, 2H).

### Example 618: (S)—N-(1-(7-(5-((Cyclopentylamino)methyl)thiophen-2-yl)quinolin-5-yl)cyclopropyl)-2-methyl-5-((1-methylazetidin-2-yl)methoxy)benzamide (Compound 752)

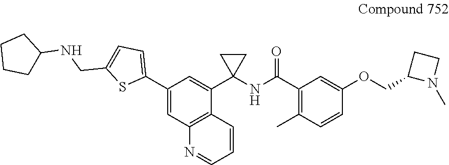 [SMILES: Cc1ccc(OC[C@@H]2CCN2C)cc1C(=O)NC1(c2cc(-c3ccc(CNC4CCCC4)s3)cc3ncccc23)CC1]

Compound 752 was synthesized according to an analogous procedure to the one described for compound 669. M+H⁺=581.4 (LCMS); ¹H NMR (400 MHZ, DMSO-d₆) δ 10.74-10.50 (m, 1H), 9.43-9.27 (m, 3H), 9.23 (br s, 1H), 9.04 (br s, 1H), 8.25 (br d, J=9.1 Hz, 2H), 7.80 (d, J=3.5 Hz, 1H), 7.74 (br d, J=4.5 Hz, 1H), 7.46 (d, J=3.0 Hz, 1H), 7.16-7.04 (m, 1H), 6.97-6.86 (m, 1H), 6.73 (d, J=2.6 Hz, 1H), 4.72-4.57 (m, 1H), 4.50-4.40 (m, 2H), 4.40-4.29 (m, 1H), 4.22 (dd, J=3.1, 11.3 Hz, 1H), 4.08-3.94 (m, 1H), 3.91-3.81 (m, 1H), 2.85-2.79 (m, 1H), 2.82 (d, J=5.0 Hz, 2H), 2.44-2.23 (m, 2H), 2.02-1.94 (m, 4H), 1.80-1.67 (m, 4H), 1.65-1.50 (m, 2H), 1.43 (br s, 2H), 1.34 (br s, 2H).

### Example 619: (S)-2-Methyl-5-((1-methylazetidin-2-yl)methoxy)-N-(1-(7-(5-(morpholinomethyl)thiophen-2-yl)quinolin-5-yl)cyclopropyl)benzamide (Compound 793)

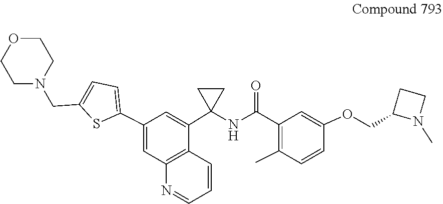 [SMILES: Cc1ccc(OC[C@@H]2CCN2C)cc1C(=O)NC1(c2cc(-c3ccc(CN4CCOCC4)s3)cc3ncccc23)CC1]

Compound 793 was synthesized according to an analogous procedure to the one described for compound 669. M+H⁺=583.2 (LCMS); ¹H NMR (400 MHZ, DMSO-d₆) δ 11.92-11.64 (m, 1H), 11.31-10.99 (m, 1H), 9.56 (br s, 1H), 9.40 (br s, 1H), 9.20 (br s, 1H), 8.44 (br s, 1H), 8.34 (br s, 1H), 8.02-7.86 (m, 2H), 7.56 (d, J=3.5 Hz, 1H), 7.09 (d, J=8.5 Hz, 1H), 6.91 (dd, J=2.6, 8.4 Hz, 1H), 6.76 (d, J=2.5 Hz, 1H), 4.6-4.61 (m, 3H), 4.42 (br dd, J=8.6, 10.8 Hz, 1H), 4.21 (br dd, J=3.1, 11.3 Hz, 1H), 3.99-3.94 (m, 4H), 3.84 (br dd, J=6.3, 9.6 Hz, 4H), 3.35 (br d, J=11.9 Hz, 2H), 2.79 (d, J=5.0 Hz, 3H), 2.36-2.23 (m, 2H), 1.98-1.93 (m, 3H), 1.46 (br s, 2H), 1.38 (br s, 2H).

### Example 620: (S)-2-Methyl-N-(1-(7-(methyl(2,2,2-trifluoroethyl)amino)quinolin-5-yl)cyclopropyl)-5-((1-methylazetidin-2-yl)methoxy)benzamide (Compound 781)

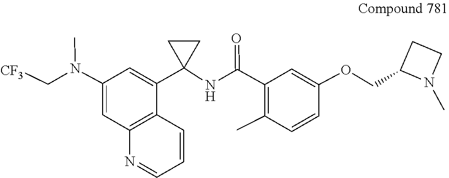 [SMILES: Cc1ccc(OC[C@@H]2CCN2C)cc1C(=O)NC1(c2cc(N(C)CC(F)(F)F)cc3ncccc23)CC1]

Compound 781 was synthesized according to an analogous procedure to the one described for compound 683. M+H⁺=513.4 (LCMS); ¹H NMR (400 MHZ, DMSO-d₆) δ11.23 (br s, 1H), 9.66-9.56 (m, 1H), 9.41-9.31 (m, 1H), 9.00 (d, J=5.5 Hz, 1H), 7.91 (d, J=2.0 Hz, 1H), 7.76 (dd, J=5.6, 8.3 Hz, 1H), 7.37 (d, J=1.9 Hz, 1H), 7.15-7.05 (m, 1H), 6.92 (dd, J=2.6, 8.4 Hz, 1H), 6.77 (d, J=2.6 Hz, 1H), 4.65 (q, J=9.1 Hz, 3H), 4.49-4.39 (m, 1H), 4.23 (dd, J=3.1, 11.2 Hz, 1H), 4.04-3.92 (m, 1H), 3.91-3.80 (m, 1H), 3.37 (br s, 2H), 3.27 (s, 3H), 2.80 (d, J=4.8 Hz, 3H), 2.40-2.26 (m, 2H), 2.02-1.96 (m, 3H), 1.48-1.31 (m, 4H).

### Example 621: (S)-2-Methyl-N-(1-(2-methyl-7-((2,2,2-trifluoroethyl)amino)quinolin-5-yl)cyclopropyl)-5-((1-methylazetidin-2-yl)methoxy)benzamide (Compound 807)

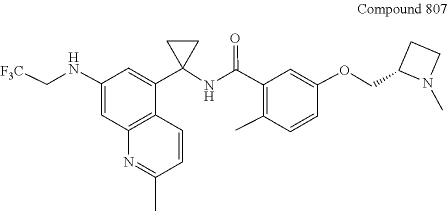 [SMILES: Cc1ccc2c(C3(NC(=O)c4cc(OC[C@@H]5CCN5C)ccc4C)CC3)cc(NCC(F)(F)F)cc2n1]

Compound 807 was synthesized according to an analogous procedure to the one described for compound 683. M+H⁺=527.3 (LCMS); ¹H NMR (400 MHZ, DMSO-d₆) 11.20-10.89 (m, 1H), 9.42 (br d, J=8.4 Hz, 1H), 9.28 (s, 1H), 7.80 (br s, 1H), 7.63 (br d, J=8.4 Hz, 1H), 7.32 (br s, 1H), 7.10 (br d, J=8.3 Hz, 1H), 6.95-6.90 (m, 1H), 6.75 (br s, 1H), 4.69-4.54 (m, 3H), 4.48-4.38 (m, 1H), 4.27-4.19 (m, 1H), 4.05-3.93 (m, 1H), 3.90-3.80 (m, 1H), 3.25 (s, 3H), 2.92-2.76 (m, 6H), 2.40-2.26 (m, 2H), 1.99 (s, 3H), 1.47-1.27 (m, 4H).

### Example 622: (S)—N-(1-(7-(Dipropylamino)quinolin-5-yl)cyclopropyl)-2-methyl-5-((1-methylazetidin-2-yl)methoxy)benzamide (Compound 771)

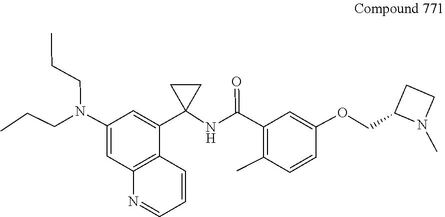 [SMILES: CCCN(CCC)c1cc(C2(NC(=O)c3cc(OC[C@@H]4CCN4C)ccc3C)CC2)c2cccnc2c1]

Compound 771 was synthesized according to an analogous procedure to the one described for compound 683. M+H⁺=501.5 (LCMS); ¹H NMR (400 MHZ, DMSO-d₆) δ 10.05-9.93 (m, 1H), 9.30 (br d, J=7.5 Hz, 1H), 9.16 (s, 1H), 8.84 (d, J=4.6 Hz, 1H), 7.72 (d, J=2.0 Hz, 1H), 7.54 (dd, J=5.8, 8.1 Hz, 1H), 7.11 (d, J=8.5 Hz, 1H), 6.97-6.89 (m, 2H), 6.76 (d, J=2.5 Hz, 1H), 4.67-4.57 (m, 1H), 4.24 (d, J=5.3 Hz, 2H), 4.03 (br d, J=4.4 Hz, 1H), 3.93-3.83 (m, 1H), 3.53-3.48 (m, 4H), 2.85 (br s, 3H), 2.43-2.28 (m, 2H), 1.97 (s, 3H), 1.74-1.63 (m, 4H), 1.38 (br s, 2H), 1.28 (br s, 2H), 0.97 (t, J=7.3 Hz, 6H).

### Example 623: (S)—N-(1-(7-(Azetidin-1-yl)quinolin-5-yl)cyclopropyl)-5-(azetidin-2-ylmethoxy)-2-methylbenzamide (Compound 722)

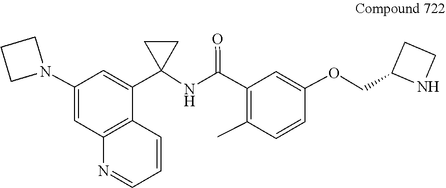 [SMILES: Cc1ccc(OC[C@@H]2CCN2)cc1C(=O)NC1(c2cc(N3CCC3)cc3ncccc23)CC1]

Compound 722 was synthesized according to an analogous procedure to the one described for compound 683. 1H NMR (400 MHZ, DMSO-d₆) δ=9.37-9.27 (m, 1H), 9.16 (s, 1H), 9.00-8.80 (m, 3H), 7.58-7.52 (m, 1H), 7.28 (d, J=2.0 Hz, 1H), 7.15-7.09 (m, 1H), 6.95-6.90 (m, 1H), 6.73 (d, J=2.6 Hz, 1H), 6.57-6.51 (m, 1H), 4.72-4.59 (m, 1H), 4.29-4.21 (m, 1H), 4.21-4.12 (m, 5H), 4.02-3.91 (m, 1H), 3.87-3.77 (m, 1H), 2.48-2.29 (m, 4H), 1.98 (s, 3H), 1.40-1.24 (m, 3H).

### Example 624: (S)—N-(1-(7-(Azetidin-1-yl)quinolin-5-yl)cyclopropyl)-2-methyl-5-((1-methylazetidin-2-yl)methoxy)benzamide (Compound 723)

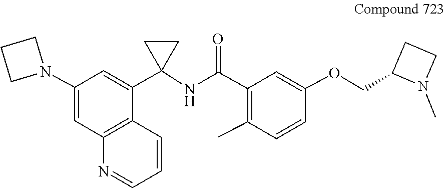 [SMILES: Cc1ccc(OC[C@@H]2CCN2C)cc1C(=O)NC1(c2cc(N3CCC3)cc3ncccc23)CC1]

Compound 723 was synthesized according to an analogous procedure to the one described for compound 683. M+H⁺=457.4. (LCMS); ¹H NMR (400 MHZ, DMSO-d₆) δ 11.01-10.13 (m, 1H), 9.10 (s, 1H), 8.85 (br d, J=7.9 Hz, 1H), 8.68 (dd, J=1.4, 4.3 Hz, 1H), 7.22 (dd, J=4.3, 8.4 Hz, 1H), 7.15 (d, J=2.1 Hz, 1H), 7.09 (d, J=8.5 Hz, 1H), 6.91 (dd, J=2.6, 8.4 Hz, 1H), 6.69 (d, J=2.4 Hz, 1H), 6.66-6.62 (m, 1H), 4.64-4.47 (m, 1H), 4.31 (br dd, J=9.1, 10.1 Hz, 1H), 4.24-4.13 (m, 1H), 3.99 (br t, J=7.3 Hz, 4H), 3.95-3.87 (m, 1H), 3.86-3.72 (m, 1H), 2.76 (br s, 3H), 2.44-2.23 (m, 4H), 1.99 (s, 3H), 1.32 (br s, 2H), 1.18 (br s, 2H).

### Example 625: (S)—N-(1-(7-(3-Methoxyazetidin-1-yl)-2-methylquinolin-5-yl)cyclopropyl)-2-methyl-5-((1-methylazetidin-2-yl)methoxy)benzamide (Compound 811)

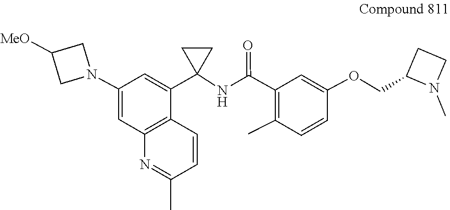 [SMILES: COC1CN(c2cc(C3(NC(=O)c4cc(OC[C@@H]5CCN5C)ccc4C)CC3)c3ccc(C)nc3c2)C1]

Compound 811 was synthesized according to an analogous procedure to the one described for compound 683. M+H⁺=501.4 (LCMS); ¹H NMR (400 MHZ, DMSO-d₆) § 15.81 (br d, J=2.1 Hz, 1H), 11.43-11.03 (m, 1H), 9.38-9.32 (m, 1H), 9.30-9.26 (m, 1H), 7.49 (d, J=8.5 Hz, 1H), 7.25 (d, J=1.8 Hz, 1H), 7.14-7.07 (m, 1H), 6.92 (dd, J=2.6, 8.4 Hz, 1H), 6.83-6.73 (m, 2H), 4.70-4.58 (m, 1H), 4.49-4.41 (m, 2H), 4.39-4.32 (m, 2H), 4.22 (dd, J=3.1, 11.2 Hz, 1H), 4.03-3.94 (m, 3H), 3.85 (br dd, J=6.8, 9.3 Hz, 1H), 3.31 (s, 3H), 2.82 (s, 3H), 2.80 (d, J=4.9 Hz, 3H), 2.38-2.26 (m, 1H), 2.40-2.26 (m, 1H), 2.00 (s, 3H), 1.37 (br s, 2H), 1.27 (br s, 2H).

### Example 626: (S)-2-Methyl-N-(1-(2-methyl-7-(3-(trifluoromethyl)azetidin-1-yl)quinolin-5-yl)cyclopropyl)-5-((1-methylazetidin-2-yl)methoxy)benzamide (Compound 810)

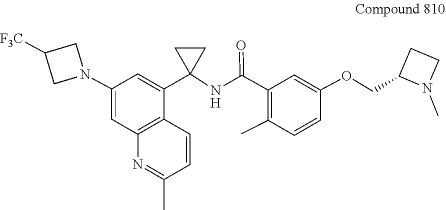 [SMILES: Cc1ccc2c(C3(NC(=O)c4cc(OC[C@@H]5CCN5C)ccc4C)CC3)cc(N3CC(C(F)(F)F)C3)cc2n1]

Compound 810 was synthesized according to an analogous procedure to the one described for compound 683. M+H⁺=539.3 (LCMS); ¹H NMR (400 MHZ, DMSO-d₆) δ 15.90-15.51 (m, 1H), 11.29-10.82 (m, 1H), 9.43-9.33 (m, 1H), 9.30-9.22 (m, 1H), 7.59-7.53 (m, 1H), 7.33-7.25 (m, 1H), 7.13-7.06 (m, 1H), 6.95-6.89 (m, 1H), 6.83-6.72 (m, 2H), 4.75-4.55 (m, 1H), 4.49-4.36 (m, 3H), 4.27-4.16 (m, 3H), 4.06-3.96 (m, 1H), 3.93-3.82 (m, 2H), 2.88-2.65 (m, 6H), 2.43-2.20 (m, 2H), 2.06-1.93 (m, 3H), 1.43-1.24 (m, 4H).

### Example 627: (S)—N-(1-(7-(3-Fluoroazetidin-1-yl)-2-methylquinolin-5-yl)cyclopropyl)-2-methyl-5-((1-methylazetidin-2-yl)methoxy)benzamide (Compound 816)

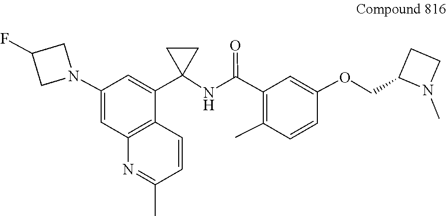 [SMILES: Cc1ccc2c(C3(NC(=O)c4cc(OC[C@@H]5CCN5C)ccc4C)CC3)cc(N3CC(F)C3)cc2n1]

Compound 816 was synthesized according to an analogous procedure to the one described for compound 683. M+H⁺=489.2 (LCMS); ¹H NMR (400 MHZ, DMSO-d₆) δ 9.32 (br d, J=8.5 Hz, 1H), 9.25-9.21 (m, 1H), 9.23 (s, 1H), 7.53 (d, J=8.4 Hz, 1H), 7.28 (d, J=1.9 Hz, 1H), 7.12 (d, J=8.5 Hz, 1H), 7.02-6.91 (m, 1H), 6.84-6.72 (m, 1H), 6.67 (s, 1H), 5.79-5.46 (m, 1H), 4.68-4.58 (m, 1H), 4.57-4.43 (m, 2H), 4.37-4.20 (m, 4H), 4.03 (dt, J=4.6, 9.5 Hz, 1H), 3.87 (q, J=9.3 Hz, 1H), 2.84 (s, 3H), 2.80 (s, 3H), 2.41-2.28 (m, 2H), 1.98 (s, 3H), 1.37 (br s, 2H), 1.29 (br s, 2H).

### Example 628: (S)-2-Methyl-N-(1-(2-methyl-7-((2,2,2-trifluoroethyl)amino)quinolin-5-yl)cyclopropyl)-5-((1-methylazetidin-2-yl)methoxy)benzamide (Compound 826)

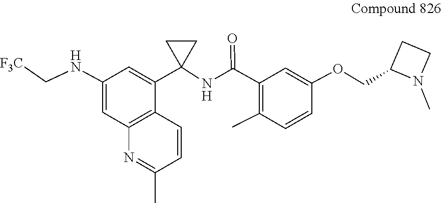 [SMILES: Cc1ccc2c(C3(NC(=O)c4cc(OC[C@@H]5CCN5C)ccc4C)CC3)cc(NCC(F)(F)F)cc2n1]

Compound 826 was synthesized according to an analogous procedure to the one described for compound 683. M+H⁺=513.4 (LCMS); ¹H NMR (400 MHZ, DMSO-d₆) 15.89 (br s, 1H), 11.25 (br d, J=4.1 Hz, 1H), 9.39-9.29 (m, 2H), 8.12 (br t, J=6.6 Hz, 1H), 7.66 (d, J=2.0 Hz, 1H), 7.57 (d, J=8.5 Hz, 1H), 7.21 (d, J=1.0 Hz, 1H), 7.09 (d, J=8.5 Hz, 1H), 6.92 (dd, J=2.7, 8.4 Hz, 1H), 6.77 (d, J=2.6 Hz, 1H), 4.72-4.59 (m, 1H), 4.46 (dd, J=8.4, 11.2 Hz, 1H), 4.24 (br d, J=3.3 Hz, 1H), 4.02-3.77 (m, 4H), 2.84 (s, 3H), 2.80 (d, J=5.0 Hz, 3H), 2.38-2.23 (m, 2H), 2.02-1.96 (m, 3H), 1.38 (br s, 2H), 1.22 (br s, 2H).

### Example 629: (S)-5-(Azetidin-2-ylmethoxy)-2-methyl-N-(1-(7-morpholinoquinolin-5-yl)cyclopropyl)benzamide (Compound 712)

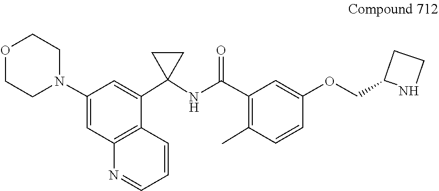 [SMILES: Cc1ccc(OC[C@@H]2CCN2)cc1C(=O)NC1(c2cc(N3CCOCC3)cc3ncccc23)CC1]

Compound 712 was synthesized according to an analogous procedure to the one described for compound 683. M+H⁺=473.3 (LCMS); ¹H NMR (400 MHZ, DMSO-d₆) δ 9.29 (br d, J=7.8 Hz, 1H), 9.16 (s, 1H), 8.94-8.89 (m, 2H), 8.88-8.80 (m, 1H), 7.85 (d, J=2.1 Hz, 1H), 7.62 (dd, J=5.3, 8.3 Hz, 1H), 7.16 (d, J=2.1 Hz, 1H), 7.11 (d, J=8.5 Hz, 1H), 6.92 (dd, J=2.7, 8.5 Hz, 1H), 6.71 (d, J=2.6 Hz, 1H), 4.70-4.64 (m, 1H), 4.29-4.19 (m, 2H), 4.16-4.12 (m, 1H), 3.93 (br d, J=7.4 Hz, 1H), 3.89-3.74 (m, 8H), 2.40-2.27 (m, 2H), 1.98 (s, 3H), 1.36 (br s, 2H), 1.32 (br s, 2H).

### Example 630: (S)-2-Methyl-5-((1-methylazetidin-2-yl)methoxy)-N-(1-(7-morpholinoquinolin-5-yl)cyclopropyl)benzamide (Compound 740)

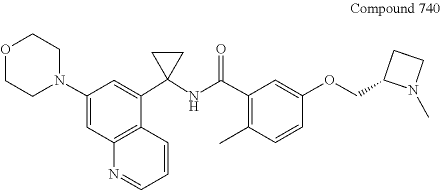 [SMILES: Cc1ccc(OC[C@@H]2CCN2C)cc1C(=O)NC1(c2cc(N3CCOCC3)cc3ncccc23)CC1]

Compound 740 was synthesized according to an analogous procedure to the one described for compound 683. M+H⁺=487.1 (LCMS); ¹H NMR (400 MHZ, DMSO-d₆) δ 10.78-10.66 (m, 1H), 9.49 (br d, J=7.9 Hz, 1H), 9.26 (s, 1H), 8.96 (d, J=4.6 Hz, 1H), 7.91 (d, J=2.1 Hz, 1H), 7.73 (dd, J=5.6, 8.3 Hz, 1H), 7.31-7.25 (m, 1H), 7.10 (d, J=8.4 Hz, 1H), 6.93 (dd, J=2.6, 8.4 Hz, 1H), 6.82-6.72 (m, 1H), 4.68-4.57 (m, 1H), 4.37 (dd, J=7.9, 11.4 Hz, 1H), 4.22 (dd, J=3.3, 11.4 Hz, 1H), 4.05-3.95 (m, 1H), 3.88-3.80 (m, 5H), 3.53-3.50 (m, 4H), 2.85-2.79 (m, 3H), 2.39-2.27 (m, 2H), 1.98 (s, 3H), 1.39 (br s, 2H), 1.34 (br s, 2H).

### Example 631: (S)-5-(Azetidin-2-ylmethoxy)-N-(1-(7-(isoxazol-4-yl)quinolin-5-yl)cyclopropyl)-2-methylbenzamide (Compound 714)

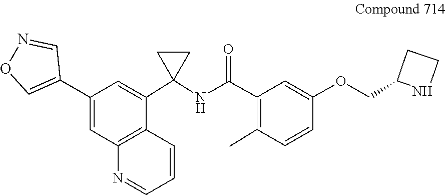 [SMILES: Cc1ccc(OC[C@@H]2CCN2)cc1C(=O)NC1(c2cc(-c3cnoc3)cc3ncccc23)CC1]

Compound 714 was synthesized according to an analogous procedure to the one described for compound 689. M+H⁺=455.1 (LCMS); ¹H NMR (400 MHZ, DMSO-d₆) δ=9.76-9.67 (m, 1H), 9.46-9.35 (m, 1H), 9.27-9.19 (m, 1H), 9.16-9.10 (m, 1H), 9.04-8.95 (m, 1H), 8.32-8.27 (m, 1H), 8.22-8.15 (m, 1H), 7.71-7.62 (m, 1H), 7.13-7.05 (m, 1H), 6.96-6.86 (m, 1H), 6.68 (d, J=2.6 Hz, 1H), 4.71-4.59 (m, 1H), 4.26-4.17 (m, 1H), 4.16-4.08 (m, 1H), 3.98-3.76 (m, 2H), 2.48-2.30 (m, 2H), 2.00-1.92 (m, 3H), 1.43-1.32 (m, 4H).

### Example 632: (S)-5-(Azetidin-2-ylmethoxy)-N-(1-(7-(imidazo[1,5-a]pyridin-1-yl)quinolin-5-yl)cyclopropyl)-2-methylbenzamide (Compound 733)

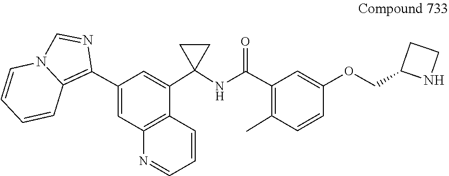 [SMILES: Cc1ccc(OC[C@@H]2CCN2)cc1C(=O)NC1(c2cc(-c3ncn4ccccc34)cc3ncccc23)CC1]

Compound 733 was synthesized according to an analogous procedure to the one described for compound 689. M+H⁺=504.3 (LCMS); ¹H NMR (400 MHZ, DMSO-d₆) δ 9.48-9.40 (m, 1H), 9.26 (s, 1H), 9.13 (br d, J=3.9 Hz, 1H), 8.91-8.76 (m, 2H), 8.73-8.67 (m, 2H), 8.54 (d, J=7.0 Hz, 1H), 8.48 (s, 1H), 8.17 (d, J=9.3 Hz, 1H), 7.89-7.80 (m, 1H), 7.23-7.15 (m, 1H), 7.11 (d, J=8.5 Hz, 1H), 6.94-6.87 (m, 2H), 6.75 (d, J=2.5 Hz, 1H), 4.65 (br d, J=5.9 Hz, 1H), 4.27-4.21 (m, 1H), 4.14 (br dd, J=3.4, 11.1 Hz, 1H), 3.96-3.90 (m, 2H), 2.47-2.27 (m, 2H), 1.99 (s, 3H), 1.47 (br s, 2H), 1.35 (br s, 2H).

### Example 633: (S)-2-Methyl-N-(1-(7-(prop-1-en-2-yl)quinolin-5-yl)cyclopropyl)-5-(pyrrolidin-2-ylmethoxy)benzamide (Compound 715)

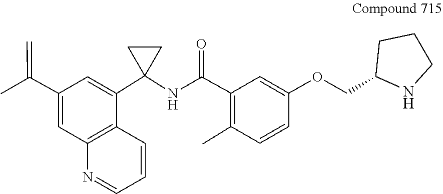 [SMILES: C=C(C)c1cc(C2(NC(=O)c3cc(OC[C@@H]4CCCN4)ccc3C)CC2)c2cccnc2c1]

Compound 715 was synthesized according to an analogous procedure to the one described for compound 713. M+H⁺=442.1 (LCMS); ¹H NMR (400 MHZ, DMSO-d₆) δ 9.36-9.27 (m, 1H), 9.23-9.16 (m, 1H), 9.10-9.01 (m, 1H), 8.23-8.18 (m, 1H), 8.05-8.00 (m, 1H), 7.83-7.74 (m, 1H), 7.12-7.05 (m, 1H), 6.95-6.86 (m, 1H), 6.73-6.61 (m, 1H), 5.83-5.75 (m, 1H), 5.45-5.39 (m, 1H), 4.22-4.10 (m, 1H), 4.03-3.93 (m, 1H), 3.88-3.77 (m, 1H), 3.23-3.13 (m, 2H), 2.30-2.22 (m, 3H), 2.14-2.03 (m, 1H), 1.99-1.84 (m, 5H), 1.73-1.61 (m, 1H), 1.43-1.37 (m, 2H), 1.33-1.26 (m, 2H).

### Example 634: (S)-2-Methyl-N-(1-(2-methyl-7-(prop-1-yn-1-yl)quinolin-5-yl)cyclopropyl)-5-((1-methylazetidin-2-yl)methoxy)benzamide (Compound 824)

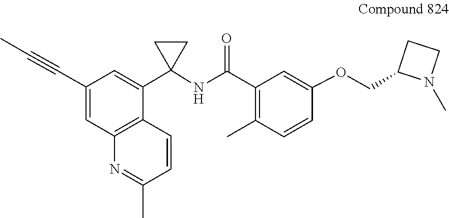 [SMILES: CC#Cc1cc(C2(NC(=O)c3cc(OC[C@@H]4CCN4C)ccc3C)CC2)c2ccc(C)nc2c1]

Compound 824 was synthesized according to an analogous procedure to the one described for compound 721. M+H⁺=454.4 (LCMS); ¹H NMR (400 MHZ, DMSO-d₆) δ 10.35-9.96 (m, 1H), 9.17 (s, 1H), 9.11 (br d, J=8.6 Hz, 1H), 7.89 (s, 1H), 7.82 (d, J=1.3 Hz, 1H), 7.65 (d, J=8.8 Hz, 1H), 7.14-7.05 (m, 1H), 6.97-6.87 (m, 1H), 6.80-6.63 (m, 1H), 4.61 (br d, J=5.4 Hz, 1H), 4.31-4.19 (m, 2H), 4.03 (br dd, J=4.6, 8.9 Hz, 1H), 3.87 (br dd, J=6.1, 9.6 Hz, 1H), 2.84 (br d, J=4.4 Hz, 3H), 2.74 (s, 3H), 2.43-2.29 (m, 2H), 2.15 (s, 3H), 1.95 (s, 3H), 1.35 (br s, 2H), 1.23 (br s, 2H).

### Example 635: (S)-2-Methyl-N-(1-(7-(methylamino)quinolin-5-yl)cyclopropyl)-5-((1-methylazetidin-2-yl)methoxy)benzamide (Compound 763)

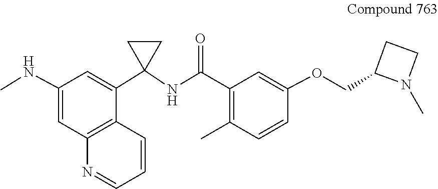

Compound 763 was synthesized according to an analogous procedure to the one described for compound 729. M+H⁺=431.3 (LCMS); ¹H NMR (400 MHZ, DMSO-d₆) δ 10.09-9.88 (m, 1H), 9.26 (br d, J=8.3 Hz, 1H), 9.15 (s, 1H), 8.79 (d, J=5.1 Hz, 1H), 7.78-7.65 (m, 1H), 7.57 (d, J=1.8 Hz, 1H), 7.52 (dd, J=5.6, 8.0 Hz, 1H), 7.11 (d, J=8.5 Hz, 1H), 6.93 (dd, J=2.6, 8.6 Hz, 1H), 6.76 (d, J=2.5 Hz, 1H), 6.66 (s, 1H), 4.68-4.55 (m, 1H), 4.24 (d, J=5.3 Hz, 2H), 4.09-3.99 (m, 1H), 3.88 (br d, J=9.9 Hz, 1H), 2.91-2.81 (m, 6H), 2.42-2.29 (m, 2H), 1.97 (s, 3H), 1.36 (br s, 2H), 1.20 (br s, 2H).

### Example 636: (S)-2-Methyl-4-(methylamino)-5-((1-methylazetidin-2-yl)methoxy)-N-(1-(7-vinylquinolin-5-yl)cyclopropyl)benzamide (Compound 745)

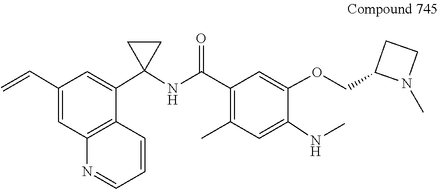 [SMILES: C=Cc1cc(C2(NC(=O)c3cc(OC[C@@H]4CCN4C)c(NC)cc3C)CC2)c2cccnc2c1]

Compound 745 was synthesized according to an analogous procedure to the one described for compound 737. M+H⁺=457.4 (LCMS); ¹H NMR (400 MHZ, DMSO-d₆) δ 10.81-10.58 (m, 1H), 9.76-9.61 (m, 1H), 9.17 (d, J=4.6 Hz, 1H), 9.00 (s, 1H), 8.25 (s, 1H), 8.12 (s, 1H), 7.96 (dd, J=5.0, 8.3 Hz, 1H), 7.15-6.99 (m, 1H), 6.87-6.73 (m, 1H), 6.41 (br s, 1H), 6.26-6.15 (m, 1H), 5.65 (d, J=11.0 Hz, 1H), 4.68-4.59 (m, 1H), 4.33 (br dd, J=5.7, 11.6 Hz, 1H), 4.19 (br dd, J=2.5, 12.0 Hz, 1H), 4.10-4.02 (m, 2H), 2.82 (s, 3H), 2.73 (s, 3H), 2.41-2.34 (m, 2H), 2.08 (s, 3H), 1.42 (br s, 2H), 1.37-1.30 (m, 2H).

### Example 637: (S)-2-Methyl-5-((1-methylazetidin-2-yl)methoxy)-N-(1-(7-((2,2,2-trifluoroethyl)amino)quinolin-5-yl)cyclopropyl)benzamide (Compound 759)

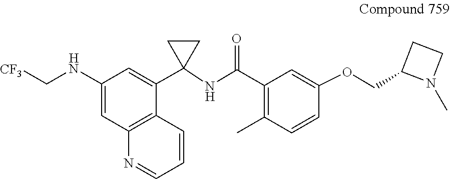 [SMILES: Cc1ccc(OC[C@@H]2CCN2C)cc1C(=O)NC1(c2cc(NCC(F)(F)F)cc3ncccc23)CC1]

Compound 759 was synthesized according to an analogous procedure to the one described for compound 743. M+H⁺=499.4 (LCMS); ¹H NMR (400 MHZ, DMSO-d₆) δ 9.05 (s, 1H), 8.85-8.73 (m, 1H), 8.66 (d, J=2.9 Hz, 1H), 7.44 (d, J=2.1 Hz, 1H), 7.24-7.19 (m, 1H), 7.03 (d, J=8.4 Hz, 1H), 6.99 (s, 1H), 6.90-6.80 (m, 2H), 6.60 (d, J=2.5 Hz, 1H), 4.14-4.00 (m, 2H), 3.86 (d, J=5.4 Hz, 2H), 3.27-3.21 (m, 2H), 2.77-2.69 (m, 1H), 2.21 (s, 3H), 1.97 (s, 3H), 1.95-1.79 (m, 2H), 1.31 (br s, 2H), 1.16-1.10 (m, 2H).

### Example 638: (S)—N-(1-(7-(Benzylamino)quinolin-5-yl)cyclopropyl)-2-methyl-5-((1-methylazetidin-2-yl)methoxy)benzamide (Compound 747)

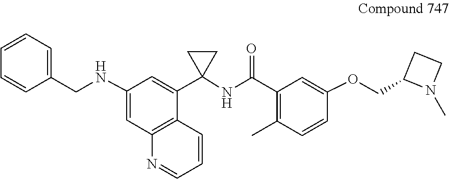 [SMILES: Cc1ccc(OC[C@@H]2CCN2C)cc1C(=O)NC1(c2cc(NCc3ccccc3)cc3ncccc23)CC1]

Compound 747 was synthesized according to an analogous procedure to the one described for compound 743. M+H⁺=507.4 (LCMS); ¹H NMR (400 MHZ, DMSO-d₆) δ 11.02-10.84 (m, 1H), 9.43-9.35 (m, 1H), 9.31-9.21 (m, 1H), 8.80-8.73 (m, 1H), 8.49-8.32 (m, 1H), 7.71 (d, J=2.0 Hz, 1H), 7.56 (dd, J=5.8, 8.2 Hz, 1H), 7.46-7.35 (m, 4H), 7.32-7.25 (m, 1H), 7.13-7.07 (m, 1H), 6.96-6.90 (m, 1H), 6.85-6.80 (m, 1H), 6.77 (d, J=2.8 Hz, 1H), 4.67-4.58 (m, 1H), 4.50-4.37 (m, 3H), 4.22 (dd, J=3.1, 11.1 Hz, 1H), 4.04-3.95 (m, 1H), 3.93-3.77 (m, 1H), 2.81 (d, J=4.6 Hz, 3H), 2.40-2.24 (m, 2H), 1.99 (s, 3H), 1.38 (br s, 2H), 1.21 (br s, 2H).

### Example 639: (S)-2-Methyl-5-((1-methylazetidin-2-yl)methoxy)-4-(methylsulfonamido)-N-(1-(7-vinylquinolin-5-yl)cyclopropyl)benzamide (Compound 760)

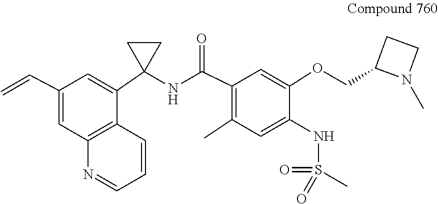 [SMILES: C=Cc1cc(C2(NC(=O)c3cc(OC[C@@H]4CCN4C)c(NS(C)(=O)=O)cc3C)CC2)c2cccnc2c1]

Compound 760 was synthesized according to an analogous procedure to the one described for compound 746. M+H⁺=521.4 (LCMS); ¹H NMR (400 MHZ, DMSO-d₆) δ 10.84 (br s, 1H), 9.62 (br d, J=8.5 Hz, 1H), 9.47-9.29 (m, 1H), 9.27-9.13 (m, 2H), 8.25 (d, J=1.3 Hz, 1H), 8.17 (s, 1H), 7.97 (dd, J=5.0, 8.5 Hz, 1H), 7.16-7.00 (m, 2H), 6.97-6.83 (m, 1H), 6.22 (d, J=17.6 Hz, 1H), 5.65 (d, J=11.0 Hz, 1H), 4.68 (br dd, J=3.4, 8.7 Hz, 1H), 4.39 (dd, J=5.8, 11.6 Hz, 1H), 4.24 (br dd, J=2.6, 11.6 Hz, 1H), 4.06-4.01 (m, 1H), 3.85 (br d, J=3.1 Hz, 1H), 2.99 (s, 3H), 2.83 (d, J=5.0 Hz, 3H), 2.40-2.33 (m, 2H), 1.98 (s, 3H), 1.44 (br s, 2H), 1.35 (br s, 2H).

### Example 640: (S)-4-Hydroxy-2-methyl-5-((1-methylazetidin-2-yl)methoxy)-N-(1-(7-vinylquinolin-5-yl)cyclopropyl)benzamide (Compound 755)

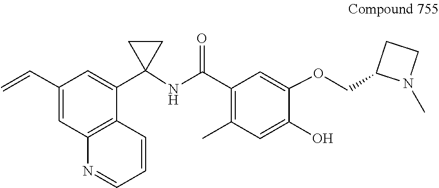 [SMILES: C=Cc1cc(C2(NC(=O)c3cc(OC[C@@H]4CCN4C)c(O)cc3C)CC2)c2cccnc2c1]

Compound 755 was synthesized according to an analogous procedure to the one described for compound 750. M+H⁺=444.2 (LCMS); ¹H NMR (400 MHZ, DMSO-d₆) δ 9.11-9.03 (m, 1H), 8.93-8.84 (m, 2H), 8.06-8.00 (m, 1H), 7.94-7.89 (m, 1H), 7.59-7.50 (m, 1H), 7.05-6.92 (m, 1H), 6.77-6.71 (m, 1H), 6.55-6.51 (m, 1H), 6.12-6.03 (m, 1H), 5.51-5.44 (m, 1H), 3.97-3.66 (m, 3H), 2.90-2.82 (m, 1H), 2.25-2.22 (m, 3H), 2.18-1.92 (m, 6H), 1.39-1.32 (m, 2H), 1.27-1.20 (m, 2H).

### Example 641: N-(1-(7-(5-(1-Hydroxyethyl)thiophen-2-yl)quinolin-5-yl)cyclopropyl)-2-methyl-5-(((S)-1-methylazetidin-2-yl)methoxy)benzamide (Compound 762)

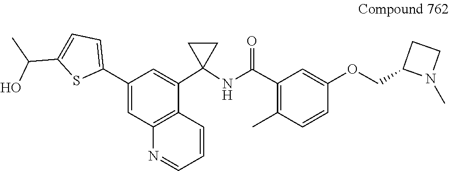 [SMILES: Cc1ccc(OC[C@@H]2CCN2C)cc1C(=O)NC1(c2cc(-c3ccc(C(C)O)s3)cc3ncccc23)CC1]

Compound 762 was synthesized according to an analogous procedure to the one described for compound 753. M+H⁺=528.3 (LCMS); ¹H NMR (400 MHZ, DMSO-d₆) δ 10.35-10.20 (m, 1H), 9.30-9.20 (m, 2H), 9.03 (br d, J=3.6 Hz, 1H), 8.20 (br d, J=18.0 Hz, 2H), 7.77-7.69 (m, 1H), 7.66 (d, J=3.6 Hz, 1H), 7.12-7.08 (m, 1H), 7.05 (d, J=3.9 Hz, 1H), 6.94-6.89 (m, 1H), 6.73 (d, J=2.6 Hz, 1H), 5.00 (q, J=6.0 Hz, 1H), 4.65-4.57 (m, 1H), 4.35-4.26 (m, 1H), 4.25-4.18 (m, 1H), 4.03-3.95 (m, 1H), 3.87-3.80 (m, 1H), 2.82 (d, J=5.0 Hz, 3H), 2.72-2.62 (m, 1H), 2.37-2.27 (m, 2H), 1.97 (s, 3H), 1.48 (d, J=6.4 Hz, 3H), 1.41 (br s, 2H), 1.37-1.31 (m, 2H).

### Example 642: 2-Methyl-5-((R)-1-((S)-1-methylazetidin-2-yl) ethoxy)-N-(1-(7-vinylquinolin-5-yl)cyclopropyl)benzamide (Compound 758)

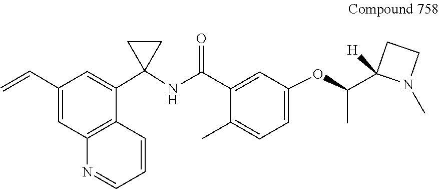 [SMILES: C=Cc1cc(C2(NC(=O)c3cc(O[C@H](C)[C@@H]4CCN4C)ccc3C)CC2)c2cccnc2c1]

Compound 758 was synthesized according to an analogous procedure to the one described for compound 757. M+H⁺=442.4 (LCMS); ¹H NMR (400 MHZ, DMSO-d₆) δ 10.91-10.63 (m, 1H), 9.47 (br s, 1H), 9.27 (s, 1H), 9.12 (br d, J=2.3 Hz, 1H), 8.20 (s, 1H), 8.10 (s, 1H), 7.94-7.83 (m, 1H), 7.11-7.01 (m, 2H), 6.96 (dd, J=2.6, 8.4 Hz, 1H), 6.78 (d, J=2.3 Hz, 1H), 6.20 (d, J=17.6 Hz, 1H), 5.62 (d, J=10.9 Hz, 1H), 4.95-4.81 (m, 1H), 4.44-4.31 (m, 1H), 3.98-3.92 (m, 1H), 3.82 (br dd, J=6.8, 9.5 Hz, 1H), 2.78 (d, J=4.9 Hz, 3H), 2.39-2.32 (m, 1H), 2.28-2.20 (m, 1H), 1.97 (s, 3H), 1.42 (br s, 2H), 1.33 (br s, 2H), 1.07 (d, J=6.1 Hz, 3H).

### Example 643: (R)—N-(1-(7-Methoxyquinolin-5-yl)cyclopropyl)-2-methyl-5-((5-methyl-5-azaspiro[2.4]heptan-6-yl)methoxy)benzamide (Compound 774)

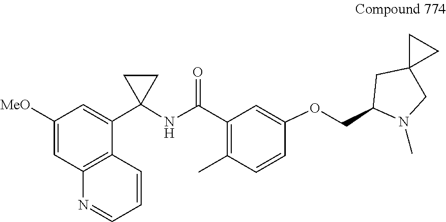 [SMILES: COc1cc(C2(NC(=O)c3cc(OC[C@H]4CC5(CC5)CN4C)ccc3C)CC2)c2cccnc2c1]

Compound 774 was synthesized according to an analogous procedure to the one described for compound 775. M+H⁺=472.3 (LCMS); ¹H NMR (400 MHZ, DMSO-d₆) δ 9.10 (s, 1H), 8.95 (d, J=8.3 Hz, 1H), 8.81 (dd, J=1.4, 4.2 Hz, 1H), 7.48-7.38 (m, 2H), 7.31 (d, J=2.5 Hz, 1H), 7.03 (d, J=8.4 Hz, 1H), 6.84 (dd, J=2.7, 8.3 Hz, 1H), 6.61 (d, J=2.6 Hz, 1H), 4.02-3.85 (m, 4H), 3.78 (dd, J=6.1, 9.6 Hz, 1H), 2.80-2.68 (m, 1H), 2.61 (s, 1H), 2.44 (d, J=8.8 Hz, 1H), 2.30 (s, 3H), 2.03-1.84 (m, 4H), 1.52 (dd, J=7.6, 12.6 Hz, 1H), 1.33 (br s, 2H), 1.19 (br s, 2H), 0.61-0.34 (m, 4H).

### Example 644: (S)-2-Methyl-5-((1-(methyl-d₃)azetidin-2-yl)methoxy)-N-(1-(7-vinylquinolin-5-yl)cyclopropyl)benzamide (Compound 788)

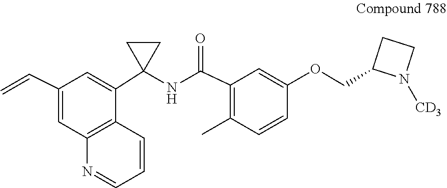 [SMILES: [2H]C([2H])([2H])N1CC[C@H]1COc1ccc(C)c(C(=O)NC2(c3cc(C=C)cc4ncccc34)CC2)c1]

Compound 788 was synthesized according to an analogous procedure to the one described for compound 782. M+H⁺=431.3 (LCMS); ¹H NMR (400 MHZ, DMSO-d₆) δ 10.45-10.30 (m, 1H), 9.34-9.27 (m, 1H), 9.22 (s, 1H), 9.05 (br d, J=3.8 Hz, 1H), 8.15 (s, 1H), 8.03 (s, 1H), 7.77 (br dd, J=4.8, 8.5 Hz, 1H), 7.13-6.99 (m, 2H), 6.92 (dd, J=2.6, 8.4 Hz, 1H), 6.72 (d, J=2.8 Hz, 1H), 6.15 (d, J=17.6 Hz, 1H), 5.57 (d, J=10.9 Hz, 1H), 4.67-4.56 (m, 1H), 4.38-4.18 (m, 2H), 4.05-3.95 (m, 1H), 3.90-3.81 (m, 1H), 2.41-2.26 (m, 2H), 1.96 (s, 3H), 1.40 (br s, 2H), 1.31 (br s, 2H).

### Example 645: (S)—N-(1-(7-Methoxy-2-methylquinolin-5-yl)cyclopropyl)-2-methyl-5-((1-(methyl-d₃)azetidin-2-yl)methoxy)benzamide (Compound 798)

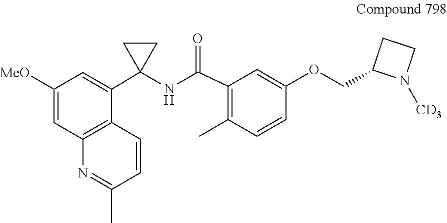 [SMILES: [2H]C([2H])([2H])N1CC[C@H]1COc1ccc(C)c(C(=O)NC2(c3cc(OC)cc4nc(C)ccc34)CC2)c1]

Compound 798 was synthesized according to an analogous procedure to the one described for compound 782. M+H⁺=449.4 (LCMS); ¹H NMR (400 MHZ, DMSO-d₆) δ 10.24-9.96 (m, 1H), 9.36-9.12 (m, 2H), 7.68 (br d, J=8.1 Hz, 1H), 7.58 (d, J=2.0 Hz, 1H), 7.40 (d, J=2.0 Hz, 1H), 7.10 (d, J=8.5 Hz, 1H), 6.97-6.87 (m, 1H), 6.81-6.69 (m, 1H), 4.74-4.55 (m, 1H), 4.23 (d, J=5.3 Hz, 2H), 4.10-4.01 (m, 1H), 3.97 (s, 3H), 3.87 (br dd, J=6.1, 9.7 Hz, 1H), 2.81 (s, 3H), 2.44-2.27 (m, 2H), 1.96 (s, 3H), 1.36 (br s, 2H), 1.26 (br s, 2H).

### Example 646: (S)-2-Methyl-5-((1-methylazetidin-2-yl)methoxy)-N-(1-(7-(N-methylmethylsulfonamido)quinolin-5-yl)cyclopropyl)benzamide (Compound 818)

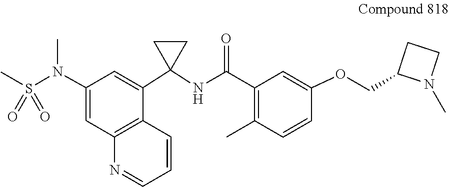

Compound 818 was synthesized according to an analogous procedure to the one described for compound 786. M+H⁺=509.2 (LCMS); ¹H NMR (400 MHZ, DMSO-d₆) δ 9.99-9.75 (m, 1H), 9.22-9.16 (m, 1H), 9.09 (d, J=8.4 Hz, 1H), 8.97 (dd, J=1.4, 4.2 Hz, 1H), 8.02 (d, J=2.3 Hz, 1H), 7.92 (d, J=2.0 Hz, 1H), 7.65 (dd, J=4.3, 8.5 Hz, 1H), 7.13-7.06 (m, 1H), 6.92 (dd, J=2.8, 8.4 Hz, 1H), 6.71 (d, J=2.6 Hz, 1H), 4.66-4.55 (m, 1H), 4.24-4.18 (m, 2H), 4.06-4.00 (m, 1H), 3.89 (br s, 1H), 3.41 (s, 3H), 3.06 (s, 3H), 2.84 (d, J=5.0 Hz, 3H), 2.43-2.24 (m, 2H), 1.97 (s, 3H), 1.39 (br s, 2H), 1.30-1.15 (m, 2H).

### Example 647: (S)-2-Methyl-N-(1-(2-methyl-7-(methylsulfonamido)quinolin-5-yl)cyclopropyl)-5-((1-methylazetidin-2-yl)methoxy)benzamide (Compound 825)

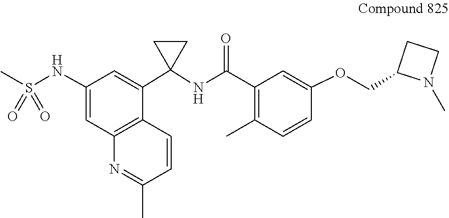

Compound 825 was synthesized according to an analogous procedure to the one described for compound 786. M+H⁺=509.4 (LCMS); ¹H NMR (400 MHZ, DMSO-d₆) δ 10.83-10.49 (m, 1H), 10.06-9.74 (m, 1H), 9.18 (s, 2H), 7.84 (s, 1H), 7.74 (br s, 1H), 7.69-7.58 (m, 1H), 7.11 (d, J=8.6 Hz, 1H), 6.93 (dd, J=2.8, 8.4 Hz, 1H), 6.80-6.69 (m, 1H), 4.67-4.58 (m, 1H), 4.23 (d, J=4.9 Hz, 2H), 4.12-3.97 (m, 1H), 3.92-3.84 (m, 1H), 3.20 (s, 3H), 2.85 (d, J=4.8 Hz, 3H), 2.78-2.70 (m, 3H), 2.42-2.29 (m, 2H), 1.96 (s, 3H), 1.39 (br s, 2H), 1.20 (br s, 2H).

### Example 648: (S)-2-Methyl-N-(1-(2-methyl-7-(N-methylmethylsulfonamido)quinolin-5-yl)cyclopropyl)-5-((1-methylazetidin-2-yl)methoxy)benzamide (Compound 819)

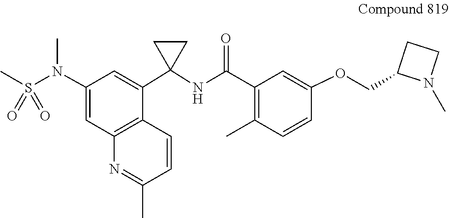

Compound 819 was synthesized according to an analogous procedure to the one described for compound 786. M+H⁺=523.2 (LCMS); ¹H NMR (400 MHZ, DMSO-d₆) δ 9.21-9.15 (m, 2H), 8.02 (d, J=2.3 Hz, 1H), 7.87 (d, J=2.0 Hz, 1H), 7.70 (d, J=8.6 Hz, 1H), 7.13-7.07 (m, 1H), 6.92 (dd, J=2.6, 8.4 Hz, 1H), 6.71 (d, J=2.8 Hz, 1H), 4.68-4.57 (m, 1H), 4.26-4.16 (m, 2H), 4.02 (dt, J=4.4, 9.7 Hz, 1H), 3.86 (q, J=9.7 Hz, 1H), 3.40 (s, 3H), 3.08 (s, 3H), 2.83 (s, 3H), 2.76 (s, 3H), 2.41-2.29 (m, 2H), 1.94 (s, 3H), 1.39 (br s, 2H), 1.25 (br s, 2H).

### Example 649: (S)—N-(1-(7-Methoxy-2-methylquinolin-5-yl)cyclopropyl-2,2,3,3-d)-2-methyl-5-((1-methylazetidin-2-yl)methoxy)benzamide (Compound 804)

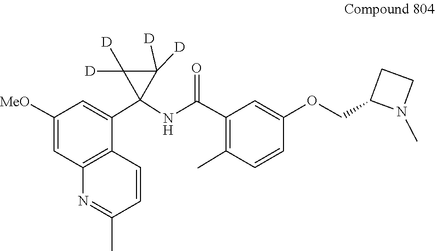 [SMILES: [2H]C1([2H])C([2H])([2H])C1(NC(=O)c1cc(OC[C@@H]2CCN2C)ccc1C)c1cc(OC)cc2nc(C)ccc12]

Compound 804 was synthesized according to an analogous procedure to the one described for compound 794. M+H⁺=450.3 (LCMS); ¹H NMR (400 MHZ, DMSO-d₆) δ 10.20-9.95 (m, 1H), 9.35 (br d, J=8.6 Hz, 1H), 9.18 (s, 1H), 7.75 (br d, J=8.4 Hz, 1H), 7.62 (br d, J=1.4 Hz, 1H), 7.42 (s, 1H), 7.11 (br d, J=8.5 Hz, 1H), 6.93 (dd, J=2.4, 8.2 Hz, 1H), 6.80-6.69 (m, 1H), 4.67-4.59 (m, 1H), 4.24 (br d, J=5.3 Hz, 2H), 4.08-3.97 (m, 4H), 3.94-3.84 (m, 1H), 2.92-2.80 (m, 6H), 2.44-2.28 (m, 2H), 1.96 (s, 3H).

### Example 650: (S)—N-(1-(7-(Fluoromethoxy)-2-methylquinolin-5-yl)cyclopropyl)-2-methyl-5-((1-methylazetidin-2-yl)methoxy)benzamide (Compound 823)

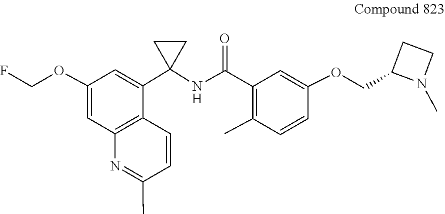 [SMILES: Cc1ccc2c(C3(NC(=O)c4cc(OC[C@@H]5CCN5C)ccc4C)CC3)cc(OCF)cc2n1]

Compound 823 was synthesized according to an analogous procedure to the one described for compound 795. M+H⁺=464.4 (LCMS); ¹H NMR (400 MHZ, DMSO-d₆) δ 11.13-10.71 (m, 1H), 9.52 (br d, J=8.1 Hz, 1H), 9.35 (s, 1H), 7.96-7.83 (m, 2H), 7.78 (d, J=2.1 Hz, 1H), 7.47 (d, J=8.0 Hz, 1H), 7.14-7.06 (m, 1H), 7.00-6.89 (m, 1H), 6.83-6.72 (m, 1H), 6.17 (s, 1H), 6.04 (s, 1H), 4.71-4.57 (m, 1H), 4.39 (dd, J=8.1, 11.3 Hz, 1H), 4.22 (dd, J=3.2, 11.2 Hz, 1H), 4.05-3.94 (m, 1H), 3.91-3.81 (m, 1H), 2.92 (s, 3H), 2.83-2.65 (m, 3H), 2.42-2.27 (m, 2H), 1.98 (s, 3H), 1.42 (br s, 2H), 1.31 (br s, 2H).

### Example 651: N-(1-(7-Methoxyquinolin-5-yl)cyclopropyl)-2-methyl-5-((2-methyl-1,2,3,4-tetrahydroisoquinolin-1-yl)methoxy)benzamide (Compound 801)

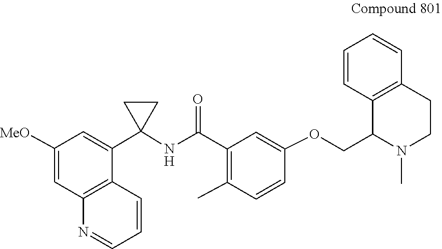 [SMILES: COc1cc(C2(NC(=O)c3cc(OCC4c5ccccc5CCN4C)ccc3C)CC2)c2cccnc2c1]

Compound 801 was synthesized according to an analogous procedure to the one described for compound 799. M+H⁺=508.3 (LCMS); ¹H NMR (400 MHZ, DMSO-d₆) δ 9.08 (s, 1H), 8.93 (d, J=8.3 Hz, 1H), 8.79 (dd, J=1.6, 4.3 Hz, 1H), 7.45 (d, J=2.6 Hz, 1H), 7.35 (dd, J=4.3, 8.5 Hz, 1H), 7.30 (d, J=2.5 Hz, 1H), 7.27-7.21 (m, 1H), 7.18-7.10 (m, 3H), 7.02 (d, J=8.5 Hz, 1H), 6.84 (dd, J=2.8, 8.4 Hz, 1H), 6.60 (d, J=2.6 Hz, 1H), 4.16 (dd, J=5.8, 10.1 Hz, 1H), 3.97 (dd, J=4.6, 10.2 Hz, 1H), 3.92 (s, 3H), 3.80 (t, J=4.9 Hz, 1H), 3.06 (ddd, J=4.9, 7.1, 11.9 Hz, 1H), 2.85-2.75 (m, 1H), 2.74-2.66 (m, 1H), 2.63-2.56 (m, 1H), 2.44 (s, 3H), 1.94 (s, 3H), 1.40-1.29 (m, 2H), 1.22-1.14 (m, 2H).

### Additional Biological Data

### List of Abbreviations

| | |
|---|---|
| BG | Back Ground |
| CPD | Compound |
| DMSO | Dimethyl Sulfoxide |
| HPE | Hundred Percent Effect |
| IC₅₀ | 50% Inhibitory Concentration |
| PLpro | Papain-like Protease |
| ZPE | Zero Percent Effect |

### Example 652: Evaluation of In Vitro Inhibitory Activity of Compounds Against SARS-CoV-2 Papain-Like Protease

Test compounds were assayed at 10 concentrations from 10 μM, in duplicate for the IC₅₀ determination. The assay buffer contained 50 mM HEPES (pH 7.5), 0.01% Triton-X 100, 0.1 mg/ml BSA and 5 mM DTT. The final concentrations of the PLpro protein and substrate in the assay were 6.25 nM and 25 μM, respectively.

Compounds were 3 folds serially diluted to 10 concentrations and added to an assay plate (384 w format) using ECHO, in duplicate wells. The final concentrations are 10 μM, 3.33 μM, 1.11 μM, 0.37 μM, 0.123 μM, 0.041 μM, 0.014 μM, 0.0046 μM, 0.0015 UM and 0.00051 μM.

20 μL of 7.8 nM of PLpro protein were added to an assay plate containing compounds using a Multidrop. The compounds and PLpro protein were pre-incubated at room temperature for 30 min. Then 5 μL of 125 μM of substrate were added to an assay plate using a Multidrop. The final concentrations of PLpro and substrate were 6.25 nM and 25 μM, respectively. For 100% inhibition control (HPE, hundred percent effect), high concentration of positive compound was added. For no inhibition control (ZPE, zero percent effect), no compound was added. The final DMSO concentration is 1%

After 60 min incubation at 25° C., the fluorescence signal (RFU) was detected using a microplate reader SpectraMax M2e (Molecular Devices) at Ex/Em=360 nm/460 nm.

The inhibitory activity (Inhibition %) was calculated using the formula below, IC₅₀ values were calculated using the Inhibition % data.

$$Inhibition \% = [ ( Sample - Average ZPE ) / ( Average HPE - Average ZPE ) ] * 100 {\%}^{\#}$$

For more potent compounds, the following PLpro enzymatic assay was used: ^{#}HEP: Hundred percent effect controls. Containing substrate+assay buffer, no compound. ZPE: Zero percent effective controls. Containing enzyme+substrate, no compound. Sample: Compound activity testing wells. Containing compound+enzyme+substrate.
PLpro enzyme: 20 μL of 1.25 nM, 1 nM final concentration
Substrate: 5 μL of 125 μM, 25 μM final concentration
Incubation time: 120 min
Certain compounds having a relatively greater potency were not tested at a top concentration of 10 μM.

IC₅₀ values of compounds were calculated with the GraphPad Prism software using the nonlinear regression model of log (inhibitor) vs. response—Variable slope (four parameters). IC₅₀ values are provided in the table below, wherein A represents an IC₅₀ value of <0.1 μM; B represents an IC₅₀ value of 0.1 to <1 μM; C represents an IC₅₀ value of 1 to <5 μM; and D represents an IC₅₀ value of ≥5 μM.

| | | | PLpro |
|---|---|---|---|
| | Example | | IC₅₀ |
| Compd ID | No. | Structure | (μM¹) |
| 172 | 1 | 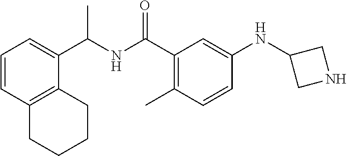 [SMILES: Cc1ccc(NC2CNC2)cc1C(=O)NC(C)c1cccc2c1CCCC2] | D |
| 168 | 2 | 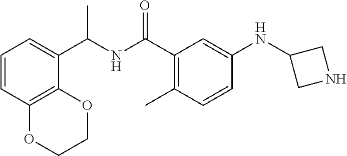 [SMILES: Cc1ccc(NC2CNC2)cc1C(=O)NC(C)c1cccc2c1OCCO2] | D |
| 155 | 3 | 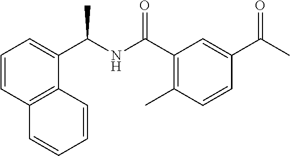 [SMILES: CC(=O)c1ccc(C)c(C(=O)N[C@H](C)c2cccc3ccccc23)c1] | D |
| 101 | 4 | 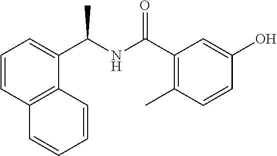 [SMILES: Cc1ccc(O)cc1C(=O)N[C@H](C)c1cccc2ccccc12] | C |
| 136 | 5 | 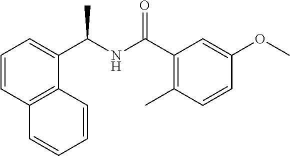 [SMILES: COc1ccc(C)c(C(=O)N[C@H](C)c2cccc3ccccc23)c1] | C |
| 132 | 6 | 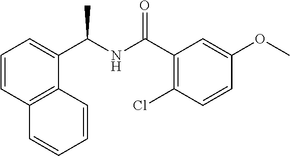 [SMILES: COc1ccc(Cl)c(C(=O)N[C@H](C)c2cccc3ccccc23)c1] | D |

¹US provisional application No. 63/352,580, to which the present application claims priority, contained a typographical error in the corresponding column heading, where the IC₅₀ data were incorrectly presented as millimolar (mM) concentrations instead of micromolar (μM) concentrations. The column heading has been corrected in the current version of the table.

| | | | PLpro |
|---|---|---|---|
| | Example | | IC₅₀ |
| Compd ID | No. | Structure | (μM¹) |
| 158 | 7 | 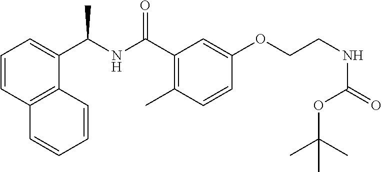 [SMILES: Cc1ccc(OCCNC(=O)OC(C)(C)C)cc1C(=O)N[C@H](C)c1cccc2ccccc12] | C |
| 159 | 8 | 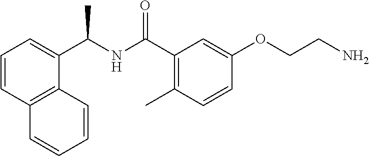 [SMILES: Cc1ccc(OCCN)cc1C(=O)N[C@H](C)c1cccc2ccccc12] | B |
| 160 | 9 | 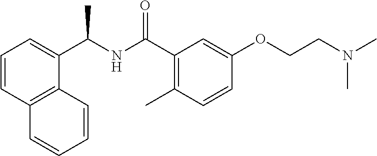 [SMILES: Cc1ccc(OCCN(C)C)cc1C(=O)N[C@H](C)c1cccc2ccccc12] | B |
| 152 | 10 | 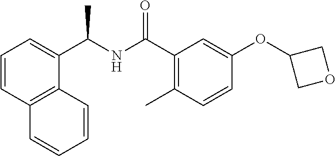 [SMILES: Cc1ccc(OC2COC2)cc1C(=O)N[C@H](C)c1cccc2ccccc12] | C |
| 141 | 11 | 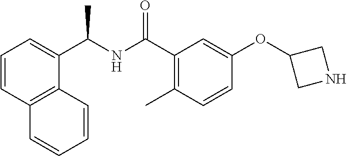 [SMILES: Cc1ccc(OC2CNC2)cc1C(=O)N[C@H](C)c1cccc2ccccc12] | B |
| 164 | 12 | 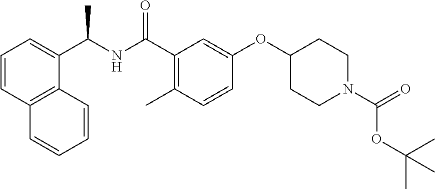 [SMILES: Cc1ccc(OC2CCN(C(=O)OC(C)(C)C)CC2)cc1C(=O)N[C@H](C)c1cccc2ccccc12] | C |
| 162 | 13 | 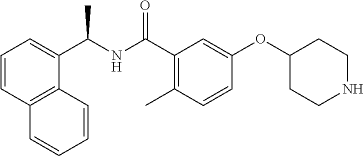 [SMILES: Cc1ccc(OC2CCNCC2)cc1C(=O)N[C@H](C)c1cccc2ccccc12] | B |
| 100 | 14 | 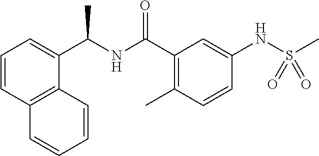 [SMILES: Cc1ccc(NS(C)(=O)=O)cc1C(=O)N[C@H](C)c1cccc2ccccc12] | D |
| 121 | 15 | 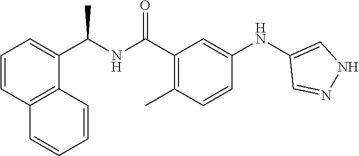 [SMILES: Cc1ccc(Nc2cn[nH]c2)cc1C(=O)N[C@H](C)c1cccc2ccccc12] | C |
| 107 | 16 | 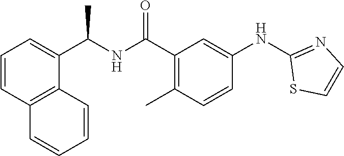 [SMILES: Cc1ccc(Nc2nccs2)cc1C(=O)N[C@H](C)c1cccc2ccccc12] | D |
| 115 | 17 | 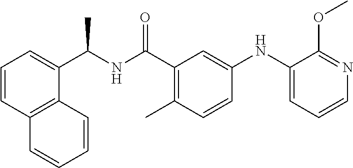 [SMILES: COc1ncccc1Nc1ccc(C)c(C(=O)N[C@H](C)c2cccc3ccccc23)c1] | C |
| 119 | 18 | 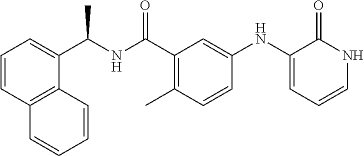 [SMILES: Cc1ccc(Nc2ccc[nH]c2=O)cc1C(=O)N[C@H](C)c1cccc2ccccc12] | C |
| 165 | 19 | 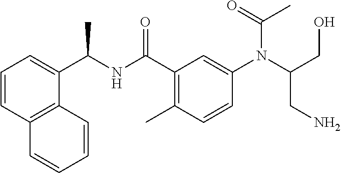 [SMILES: CC(=O)N(c1ccc(C)c(C(=O)N[C@H](C)c2cccc3ccccc23)c1)C(CN)CO] | D |
| 153 | 20 | 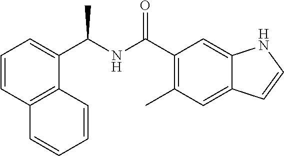 [SMILES: Cc1cc2cc[nH]c2cc1C(=O)N[C@H](C)c1cccc2ccccc12] | C |
| 189 | 21 | 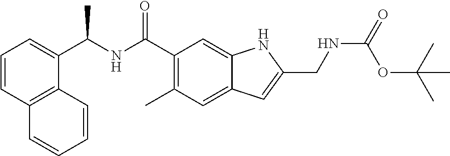 [SMILES: Cc1cc2cc(CNC(=O)OC(C)(C)C)[nH]c2cc1C(=O)N[C@H](C)c1cccc2ccccc12] | D |
| 148 | 22 | 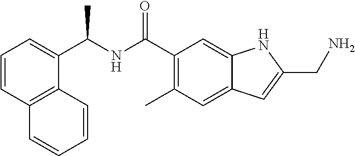 [SMILES: Cc1cc2cc(CN)[nH]c2cc1C(=O)N[C@H](C)c1cccc2ccccc12] | B |
| 156 | 23 | 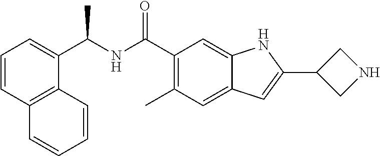 [SMILES: Cc1cc2cc(C3CNC3)[nH]c2cc1C(=O)N[C@H](C)c1cccc2ccccc12] | C |
| 192 | 24 | 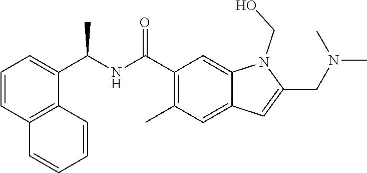 [SMILES: Cc1cc2cc(CN(C)C)n(CO)c2cc1C(=O)N[C@H](C)c1cccc2ccccc12] | B |
| 206 | 25 | 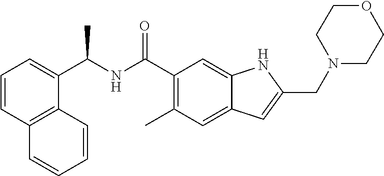 [SMILES: Cc1cc2cc(CN3CCOCC3)[nH]c2cc1C(=O)N[C@H](C)c1cccc2ccccc12] | C |
| 125 | 26 | 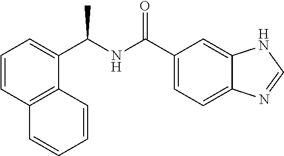 [SMILES: C[C@@H](NC(=O)c1ccc2nc[nH]c2c1)c1cccc2ccccc12] | D |
| 102 | 27 | 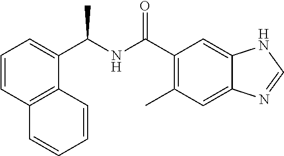 [SMILES: Cc1cc2nc[nH]c2cc1C(=O)N[C@H](C)c1cccc2ccccc12] | C |
| 128 | 28 | 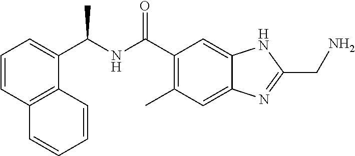 [SMILES: Cc1cc2nc(CN)[nH]c2cc1C(=O)N[C@H](C)c1cccc2ccccc12] | B |
| 131 | 29 | 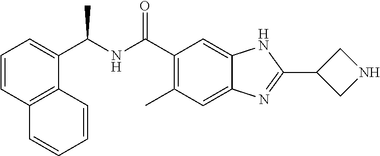 [SMILES: Cc1cc2nc(C3CNC3)[nH]c2cc1C(=O)N[C@H](C)c1cccc2ccccc12] | C |
| 124 | 30 | 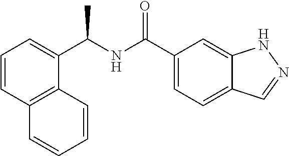 [SMILES: C[C@@H](NC(=O)c1ccc2cn[nH]c2c1)c1cccc2ccccc12] | D |
| 142 | 31 | 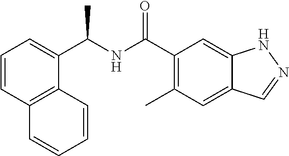 [SMILES: Cc1cc2cn[nH]c2cc1C(=O)N[C@H](C)c1cccc2ccccc12] | B |
| 150 | 32 | 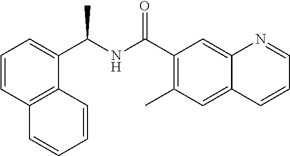 [SMILES: Cc1cc2cccnc2cc1C(=O)N[C@H](C)c1cccc2ccccc12] | C |
| 167 | 33 | 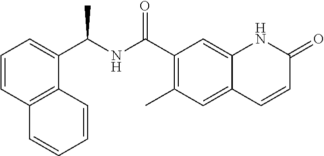 [SMILES: Cc1cc2ccc(=O)[nH]c2cc1C(=O)N[C@H](C)c1cccc2ccccc12] | D |
| 145 | 34 | 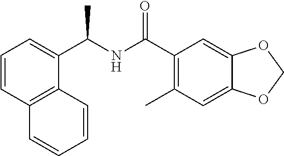 [SMILES: Cc1cc2c(cc1C(=O)N[C@H](C)c1cccc3ccccc13)OCO2] | C |
| 163 | 35 | 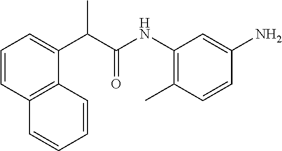 [SMILES: Cc1ccc(N)cc1NC(=O)C(C)c1cccc2ccccc12] | D |
| 195 | 36 | 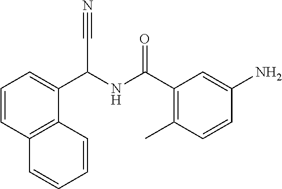 [SMILES: Cc1ccc(N)cc1C(=O)NC(C#N)c1cccc2ccccc12] | D |
| 200 | 37 | 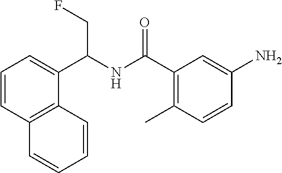 [SMILES: Cc1ccc(N)cc1C(=O)NC(CF)c1cccc2ccccc12] | C |
| 185 | 38 | 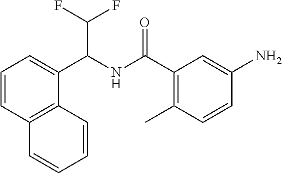 [SMILES: Cc1ccc(N)cc1C(=O)NC(c1cccc2ccccc12)C(F)F] | C |
| 183 | 39 | 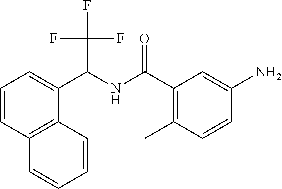 [SMILES: Cc1ccc(N)cc1C(=O)NC(c1cccc2ccccc12)C(F)(F)F] | C |
| 191 | 40 | 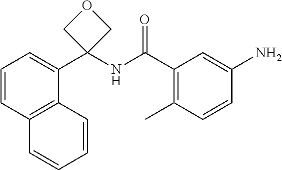 [SMILES: Cc1ccc(N)cc1C(=O)NC1(c2cccc3ccccc23)COC1] | B |
| 281 | 41 | 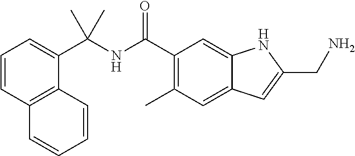 [SMILES: Cc1cc2cc(CN)[nH]c2cc1C(=O)NC(C)(C)c1cccc2ccccc12] | D |
| 241 | 42 | 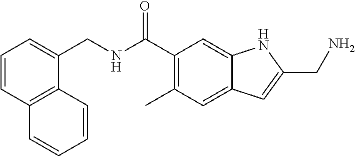 [SMILES: Cc1cc2cc(CN)[nH]c2cc1C(=O)NCc1cccc2ccccc12] | D |
| 147 | 43 | 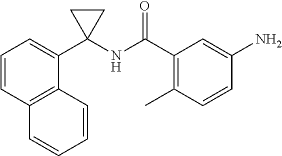 [SMILES: Cc1ccc(N)cc1C(=O)NC1(c2cccc3ccccc23)CC1] | B |
| 219 | 44 | 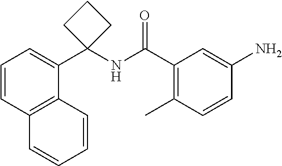 [SMILES: Cc1ccc(N)cc1C(=O)NC1(c2cccc3ccccc23)CCC1] | D |
| 177 | 45 | 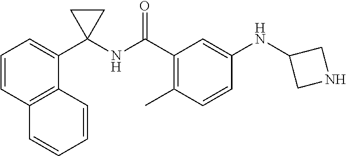 [SMILES: Cc1ccc(NC2CNC2)cc1C(=O)NC1(c2cccc3ccccc23)CC1] | B |
| 313 | 46 | 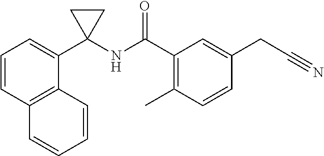 [SMILES: Cc1ccc(CC#N)cc1C(=O)NC1(c2cccc3ccccc23)CC1] | B |
| 378 | 47 | 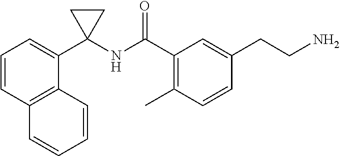 [SMILES: Cc1ccc(CCN)cc1C(=O)NC1(c2cccc3ccccc23)CC1] | A |
| 257 | 48 | 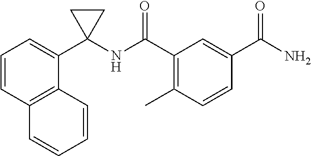 [SMILES: Cc1ccc(C(N)=O)cc1C(=O)NC1(c2cccc3ccccc23)CC1] | B |
| 312 | 49 | 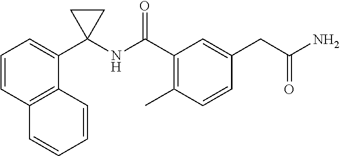 [SMILES: Cc1ccc(CC(N)=O)cc1C(=O)NC1(c2cccc3ccccc23)CC1] | B |
| 398 | 50 | 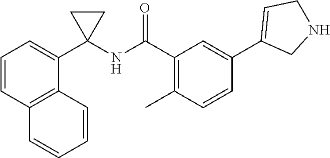 [SMILES: Cc1ccc(C2=CCNC2)cc1C(=O)NC1(c2cccc3ccccc23)CC1] | A |
| 399 | 51 | 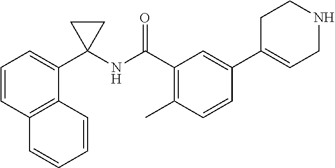 [SMILES: Cc1ccc(C2=CCNCC2)cc1C(=O)NC1(c2cccc3ccccc23)CC1] | A |
| 373 | 52 | 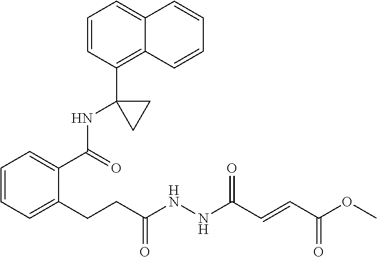 [SMILES: COC(=O)/C=C/C(=O)NNC(=O)CCc1ccccc1C(=O)NC1(c2cccc3ccccc23)CC1] | A |
| 215 | 53 | 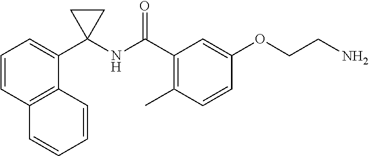 [SMILES: Cc1ccc(OCCN)cc1C(=O)NC1(c2cccc3ccccc23)CC1] | A |
| 211 | 54 | 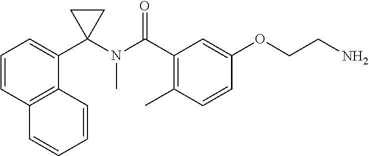 [SMILES: Cc1ccc(OCCN)cc1C(=O)N(C)C1(c2cccc3ccccc23)CC1] | D |
| 316 | 55 | 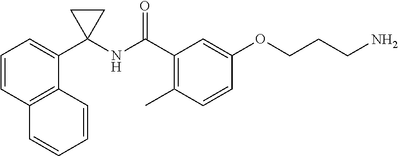 [SMILES: Cc1ccc(OCCCN)cc1C(=O)NC1(c2cccc3ccccc23)CC1] | B |
| 233 | 56 | 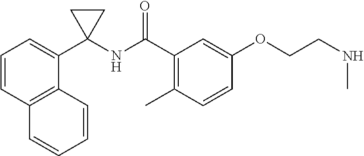 [SMILES: CNCCOc1ccc(C)c(C(=O)NC2(c3cccc4ccccc34)CC2)c1] | A |
| 386 | 57 | 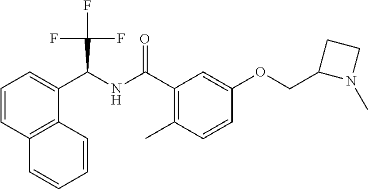 [SMILES: Cc1ccc(OCC2CCN2C)cc1C(=O)N[C@@H](c1cccc2ccccc12)C(F)(F)F] | A |
| 214 | 58 | 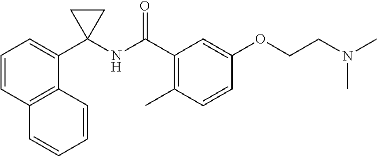 [SMILES: Cc1ccc(OCCN(C)C)cc1C(=O)NC1(c2cccc3ccccc23)CC1] | A |
| 351 | 59 | 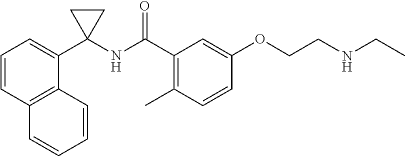 [SMILES: CCNCCOc1ccc(C)c(C(=O)NC2(c3cccc4ccccc34)CC2)c1] | A |
| 247 | 60 | 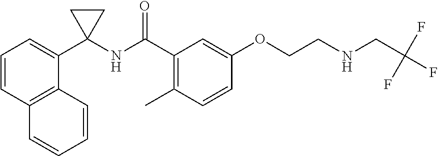 [SMILES: Cc1ccc(OCCNCC(F)(F)F)cc1C(=O)NC1(c2cccc3ccccc23)CC1] | B |
| 362 | 61 | 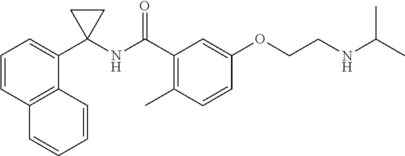 [SMILES: Cc1ccc(OCCNC(C)C)cc1C(=O)NC1(c2cccc3ccccc23)CC1] | A |
| 361 | 62 | 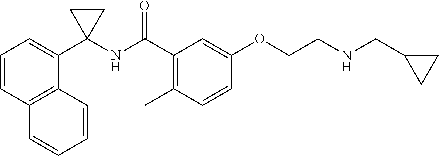 [SMILES: Cc1ccc(OCCNCC2CC2)cc1C(=O)NC1(c2cccc3ccccc23)CC1] | A |
| 315 | 63 | 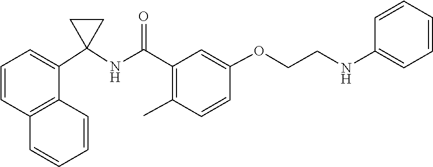 [SMILES: Cc1ccc(OCCNc2ccccc2)cc1C(=O)NC1(c2cccc3ccccc23)CC1] | C |
| 360 | 64 | 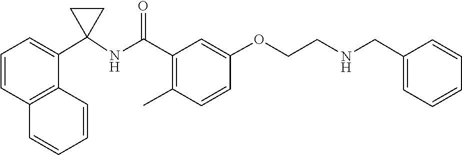 [SMILES: Cc1ccc(OCCNCc2ccccc2)cc1C(=O)NC1(c2cccc3ccccc23)CC1] | A |
| 359 | 65 | 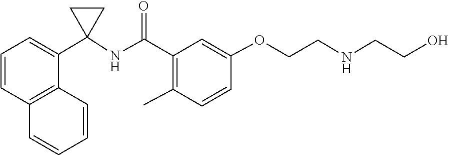 [SMILES: Cc1ccc(OCCNCCO)cc1C(=O)NC1(c2cccc3ccccc23)CC1] | A |
| 358 | 66 | 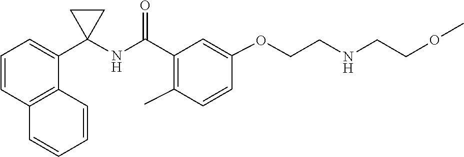 [SMILES: COCCNCCOc1ccc(C)c(C(=O)NC2(c3cccc4ccccc34)CC2)c1] | A |
| 300 | 67 | 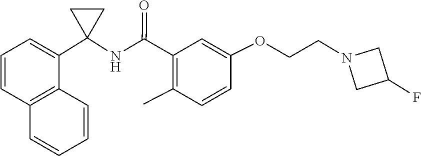 [SMILES: Cc1ccc(OCCN2CC(F)C2)cc1C(=O)NC1(c2cccc3ccccc23)CC1] | A |
| 254 | 68 | 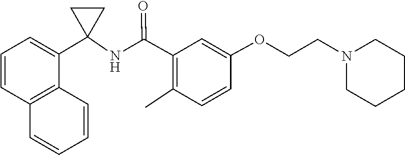 [SMILES: Cc1ccc(OCCN2CCCCC2)cc1C(=O)NC1(c2cccc3ccccc23)CC1] | A |
| 284 | 69 | 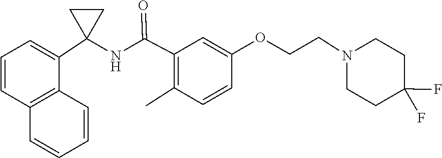 [SMILES: Cc1ccc(OCCN2CCC(F)(F)CC2)cc1C(=O)NC1(c2cccc3ccccc23)CC1] | B |
| 209 | 70 | 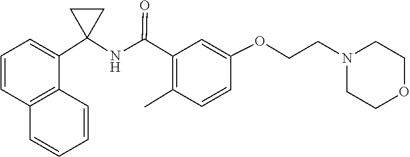 [SMILES: Cc1ccc(OCCN2CCOCC2)cc1C(=O)NC1(c2cccc3ccccc23)CC1] | B |
| 324 | 71 | 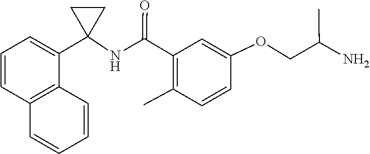 [SMILES: Cc1ccc(OCC(C)N)cc1C(=O)NC1(c2cccc3ccccc23)CC1] | A |
| 343 | 72 | 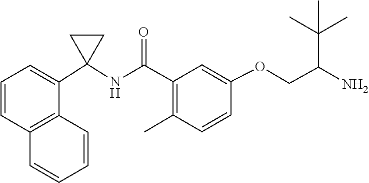 [SMILES: Cc1ccc(OCC(N)C(C)(C)C)cc1C(=O)NC1(c2cccc3ccccc23)CC1] | A |
| 400 | 73 | 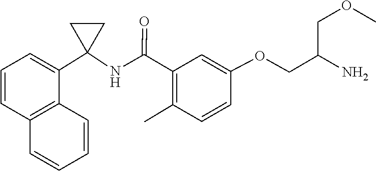 [SMILES: COCC(N)COc1ccc(C)c(C(=O)NC2(c3cccc4ccccc34)CC2)c1] | A |
| 367 | 74 | 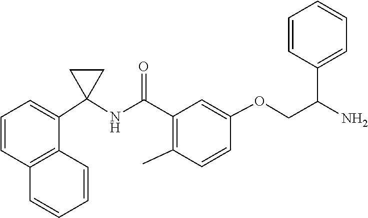 [SMILES: Cc1ccc(OCC(N)c2ccccc2)cc1C(=O)NC1(c2cccc3ccccc23)CC1] | A |
| 322 | 75 | 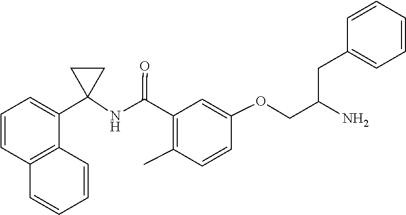 [SMILES: Cc1ccc(OCC(N)Cc2ccccc2)cc1C(=O)NC1(c2cccc3ccccc23)CC1] | A |
| 352 | 76 | 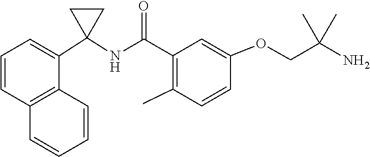 [SMILES: Cc1ccc(OCC(C)(C)N)cc1C(=O)NC1(c2cccc3ccccc23)CC1] | A |
| 256 | 77 | 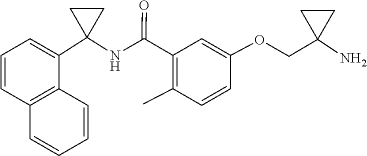 [SMILES: Cc1ccc(OCC2(N)CC2)cc1C(=O)NC1(c2cccc3ccccc23)CC1] | A |
| 274 | 78 | 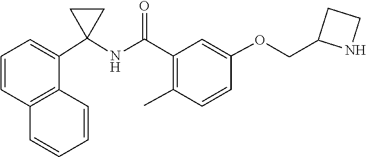 [SMILES: Cc1ccc(OCC2CCN2)cc1C(=O)NC1(c2cccc3ccccc23)CC1] | A |
| 310 | 79 | 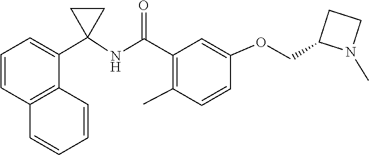 [SMILES: Cc1ccc(OC[C@@H]2CCN2C)cc1C(=O)NC1(c2cccc3ccccc23)CC1] | A |
| 309 | 80 | 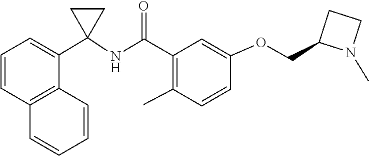 [SMILES: Cc1ccc(OC[C@H]2CCN2C)cc1C(=O)NC1(c2cccc3ccccc23)CC1] | A |
| 387 | 81 | 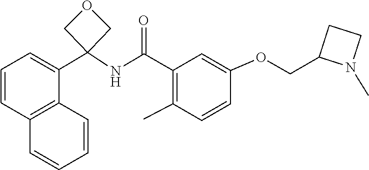 [SMILES: Cc1ccc(OCC2CCN2C)cc1C(=O)NC1(c2cccc3ccccc23)COC1] | A |
| 349 | 82 | 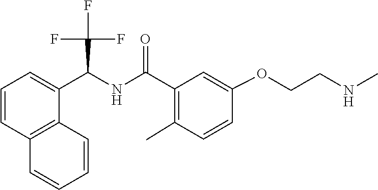 [SMILES: CNCCOc1ccc(C)c(C(=O)N[C@@H](c2cccc3ccccc23)C(F)(F)F)c1] | B |
| 314 | 83 | 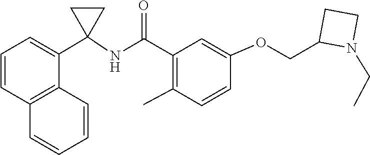 [SMILES: CCN1CCC1COc1ccc(C)c(C(=O)NC2(c3cccc4ccccc34)CC2)c1] | A |
| 396 | 84 | 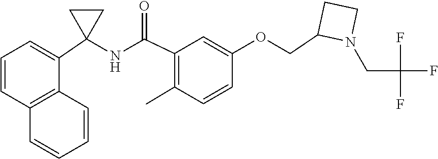 [SMILES: Cc1ccc(OCC2CCN2CC(F)(F)F)cc1C(=O)NC1(c2cccc3ccccc23)CC1] | C |
| 331 | 85 | 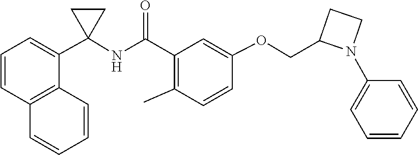 [SMILES: Cc1ccc(OCC2CCN2c2ccccc2)cc1C(=O)NC1(c2cccc3ccccc23)CC1] | D |
| 330 | 86 | 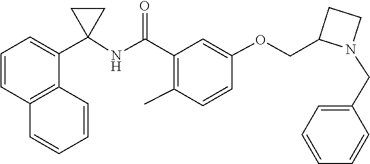 [SMILES: Cc1ccc(OCC2CCN2Cc2ccccc2)cc1C(=O)NC1(c2cccc3ccccc23)CC1] | A |
| 323 | 87 | 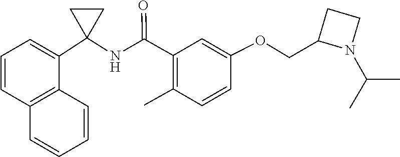 [SMILES: Cc1ccc(OCC2CCN2C(C)C)cc1C(=O)NC1(c2cccc3ccccc23)CC1] | A |
| 332 | 88 | 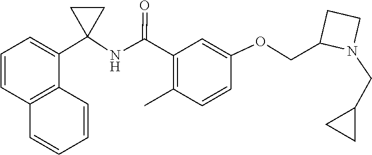 [SMILES: Cc1ccc(OCC2CCN2CC2CC2)cc1C(=O)NC1(c2cccc3ccccc23)CC1] | A |
| 348 | 89 | 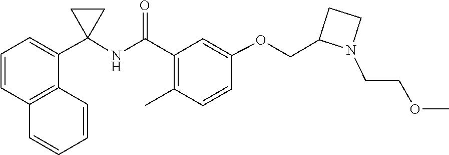 [SMILES: COCCN1CCC1COc1ccc(C)c(C(=O)NC2(c3cccc4ccccc34)CC2)c1] | A |
| 341 | 90 | 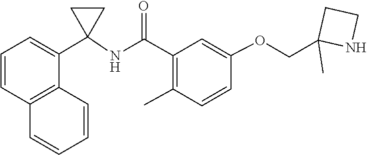 [SMILES: Cc1ccc(OCC2(C)CCN2)cc1C(=O)NC1(c2cccc3ccccc23)CC1] | A |
| 394 | 91 | 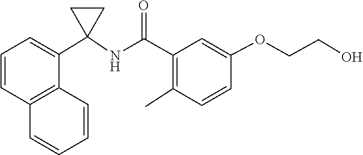 [SMILES: Cc1ccc(OCCO)cc1C(=O)NC1(c2cccc3ccccc23)CC1] | A |
| 393 | 92 | 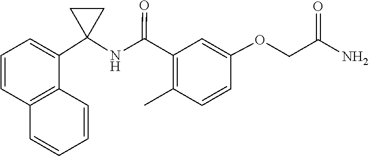 [SMILES: Cc1ccc(OCC(N)=O)cc1C(=O)NC1(c2cccc3ccccc23)CC1] | B |
| 285 | 93 | 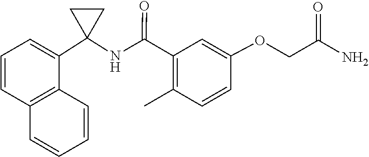 [SMILES: Cc1ccc(OCC(N)=O)cc1C(=O)NC1(c2cccc3ccccc23)CC1] | B |
| 171 | 94 | 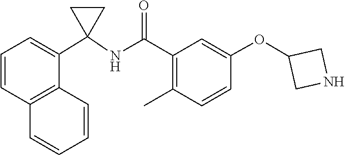 [SMILES: Cc1ccc(OC2CNC2)cc1C(=O)NC1(c2cccc3ccccc23)CC1] | B |
| 265 | 95 | 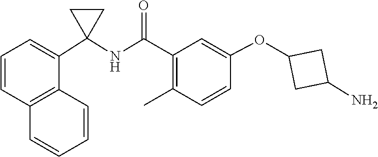 [SMILES: Cc1ccc(OC2CC(N)C2)cc1C(=O)NC1(c2cccc3ccccc23)CC1] | B |
| 263 | 96 | 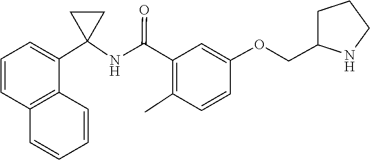 [SMILES: Cc1ccc(OCC2CCCN2)cc1C(=O)NC1(c2cccc3ccccc23)CC1] | A |
| 283 | 97 | 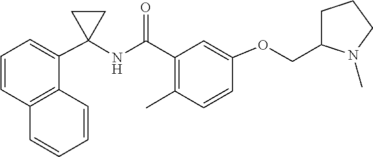 [SMILES: Cc1ccc(OCC2CCCN2C)cc1C(=O)NC1(c2cccc3ccccc23)CC1] | A |
| 268 | 98 | 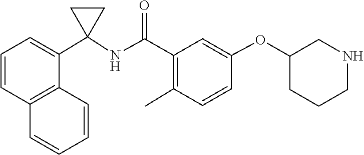 [SMILES: Cc1ccc(OC2CCCNC2)cc1C(=O)NC1(c2cccc3ccccc23)CC1] | B |
| 289 | 99 | 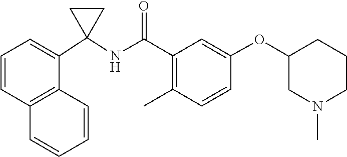 [SMILES: Cc1ccc(OC2CCCN(C)C2)cc1C(=O)NC1(c2cccc3ccccc23)CC1] | B |
| 339 | 100 | 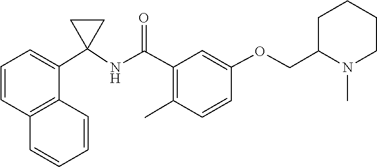 [SMILES: Cc1ccc(OCC2CCCCN2C)cc1C(=O)NC1(c2cccc3ccccc23)CC1] | A |
| 288 | 101 | 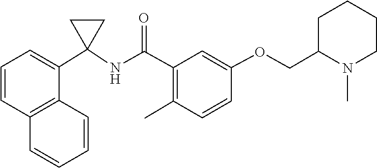 [SMILES: Cc1ccc(OCC2CCCCN2C)cc1C(=O)NC1(c2cccc3ccccc23)CC1] | B |
| 293 | 101 | 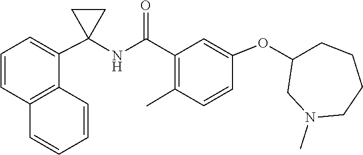 [SMILES: Cc1ccc(OC2CCCCN(C)C2)cc1C(=O)NC1(c2cccc3ccccc23)CC1] | B |
| 286 | 102 | 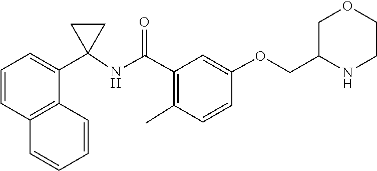 [SMILES: Cc1ccc(OCC2COCCN2)cc1C(=O)NC1(c2cccc3ccccc23)CC1] | A |
| 302 | 103 | 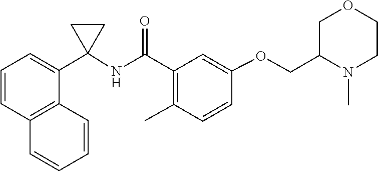 [SMILES: Cc1ccc(OCC2COCCN2C)cc1C(=O)NC1(c2cccc3ccccc23)CC1] | B |
| 376 | 105 | 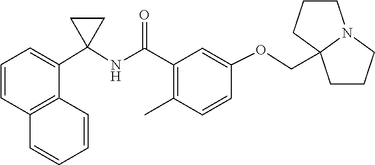 [SMILES: Cc1ccc(OCC23CCCN2CCC3)cc1C(=O)NC1(c2cccc3ccccc23)CC1] | A |
| 303 | 106 | 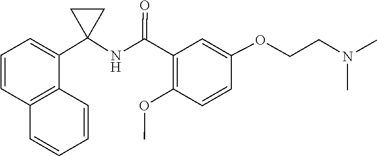 [SMILES: COc1ccc(OCCN(C)C)cc1C(=O)NC1(c2cccc3ccccc23)CC1] | D |
| 269 | 107 | 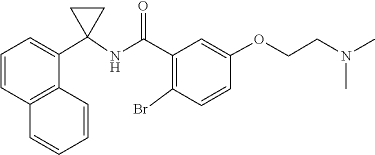 [SMILES: CN(C)CCOc1ccc(Br)c(C(=O)NC2(c3cccc4ccccc34)CC2)c1] | B |
| 337 | 108 | 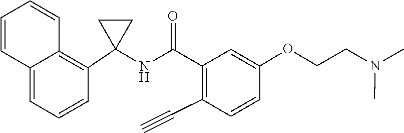 [SMILES: C#Cc1ccc(OCCN(C)C)cc1C(=O)NC1(c2cccc3ccccc23)CC1] | D |
| 353 | 109 | 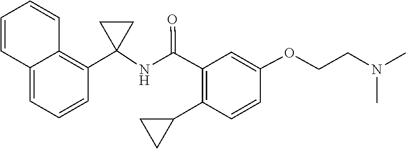 [SMILES: CN(C)CCOc1ccc(C2CC2)c(C(=O)NC2(c3cccc4ccccc34)CC2)c1] | D |
| 311 | 110 | 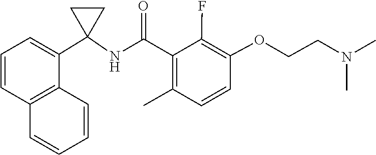 [SMILES: Cc1ccc(OCCN(C)C)c(F)c1C(=O)NC1(c2cccc3ccccc23)CC1] | B |
| 216 | 111 | 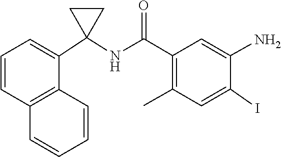 [SMILES: Cc1cc(I)c(N)cc1C(=O)NC1(c2cccc3ccccc23)CC1] | B |
| 369 | 112 | 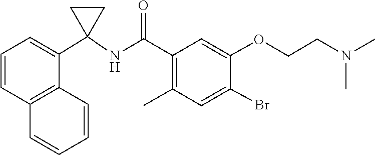 [SMILES: Cc1cc(Br)c(OCCN(C)C)cc1C(=O)NC1(c2cccc3ccccc23)CC1] | A |
| 366 | 113 | 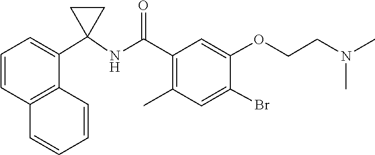 [SMILES: Cc1cc(Br)c(OCCN(C)C)cc1C(=O)NC1(c2cccc3ccccc23)CC1] | B |
| 350 | 114 | 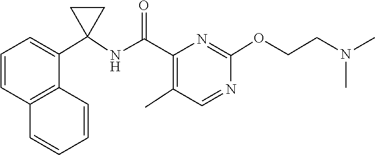 [SMILES: Cc1cnc(OCCN(C)C)nc1C(=O)NC1(c2cccc3ccccc23)CC1] | C |
| 275 | 115 | 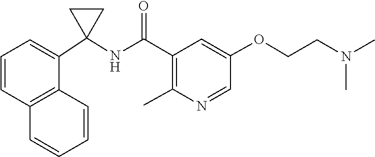 [SMILES: Cc1ncc(OCCN(C)C)cc1C(=O)NC1(c2cccc3ccccc23)CC1] | C |
| 199 | 116 | 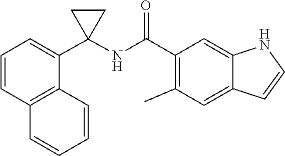 [SMILES: Cc1cc2cc[nH]c2cc1C(=O)NC1(c2cccc3ccccc23)CC1] | B |
| 240 | 117 | 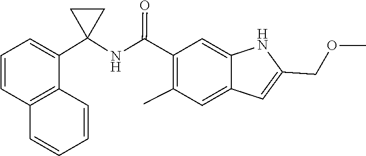 [SMILES: COCc1cc2cc(C)c(C(=O)NC3(c4cccc5ccccc45)CC3)cc2[nH]1] | B |
| 180 | 118 | 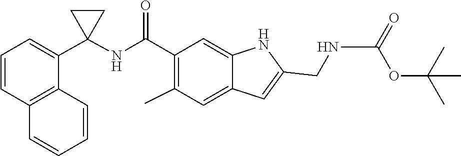 [SMILES: Cc1cc2cc(CNC(=O)OC(C)(C)C)[nH]c2cc1C(=O)NC1(c2cccc3ccccc23)CC1] | B |
| 170 | 119 | 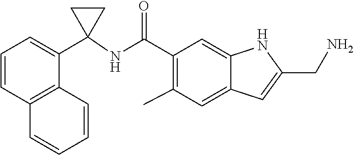 [SMILES: Cc1cc2cc(CN)[nH]c2cc1C(=O)NC1(c2cccc3ccccc23)CC1] | A |
| 248 | 120 | 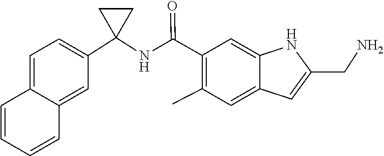 [SMILES: Cc1cc2cc(CN)[nH]c2cc1C(=O)NC1(c2ccc3ccccc3c2)CC1] | C |
| 246 | 121 | 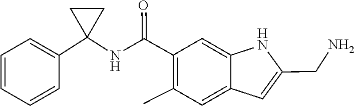 [SMILES: Cc1cc2cc(CN)[nH]c2cc1C(=O)NC1(c2ccccc2)CC1] | D |
| 244 | 122 | 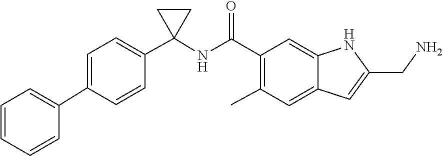 [SMILES: Cc1cc2cc(CN)[nH]c2cc1C(=O)NC1(c2ccc(-c3ccccc3)cc2)CC1] | D |
| 232 | 123 | 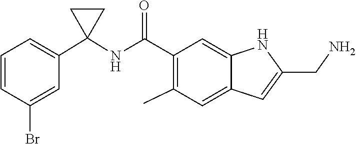 [SMILES: Cc1cc2cc(CN)[nH]c2cc1C(=O)NC1(c2cccc(Br)c2)CC1] | C |
| 221 | 124 | 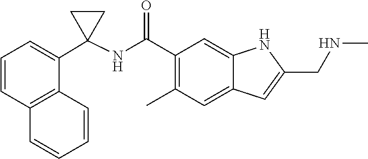 [SMILES: CNCc1cc2cc(C)c(C(=O)NC3(c4cccc5ccccc45)CC3)cc2[nH]1] | A |
| 173 | 125 | 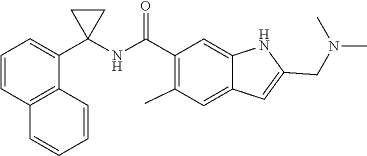 [SMILES: Cc1cc2cc(CN(C)C)[nH]c2cc1C(=O)NC1(c2cccc3ccccc23)CC1] | B |
| 273 | 126 | 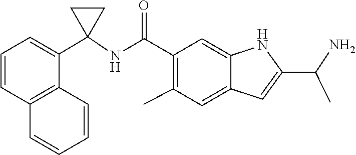 [SMILES: Cc1cc2cc(C(C)N)[nH]c2cc1C(=O)NC1(c2cccc3ccccc23)CC1] | A |
| 260 | 127 | 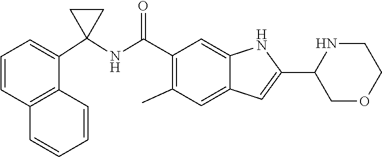 [SMILES: Cc1cc2cc(C3COCCN3)[nH]c2cc1C(=O)NC1(c2cccc3ccccc23)CC1] | B |
| 202 | 128 | 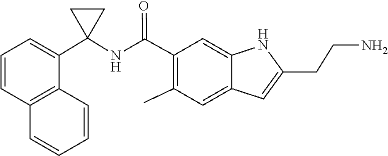 [SMILES: Cc1cc2cc(CCN)[nH]c2cc1C(=O)NC1(c2cccc3ccccc23)CC1] | B |
| 222 | 129 | 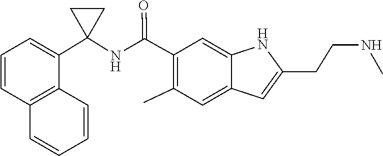 [SMILES: CNCCc1cc2cc(C)c(C(=O)NC3(c4cccc5ccccc45)CC3)cc2[nH]1] | B |
| 213 | 130 | 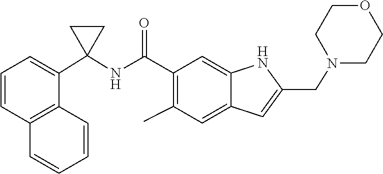 [SMILES: Cc1cc2cc(CN3CCOCC3)[nH]c2cc1C(=O)NC1(c2cccc3ccccc23)CC1] | C |
| 229 | 131 | 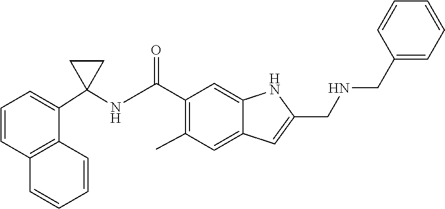 [SMILES: Cc1cc2cc(CNCc3ccccc3)[nH]c2cc1C(=O)NC1(c2cccc3ccccc23)CC1] | A |
| 204 | 132 | 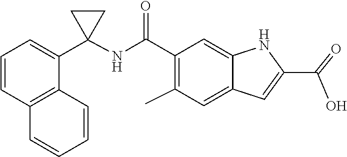 [SMILES: Cc1cc2cc(C(=O)O)[nH]c2cc1C(=O)NC1(c2cccc3ccccc23)CC1] | B |
| 205 | 133 | 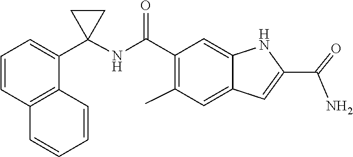 [SMILES: Cc1cc2cc(C(N)=O)[nH]c2cc1C(=O)NC1(c2cccc3ccccc23)CC1] | B |
| 225 | 134 | 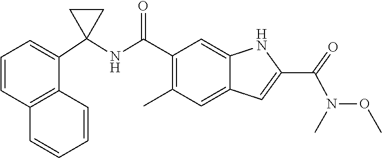 [SMILES: CON(C)C(=O)c1cc2cc(C)c(C(=O)NC3(c4cccc5ccccc45)CC3)cc2[nH]1] | B |
| 226 | 135 | 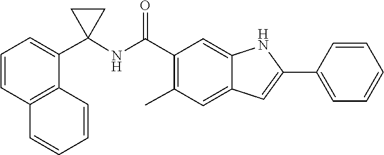 [SMILES: Cc1cc2cc(-c3ccccc3)[nH]c2cc1C(=O)NC1(c2cccc3ccccc23)CC1] | D |
| 234 | 136 | 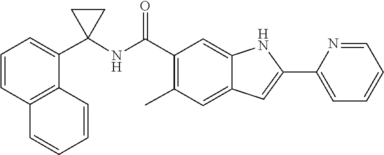 [SMILES: Cc1cc2cc(-c3ccccn3)[nH]c2cc1C(=O)NC1(c2cccc3ccccc23)CC1] | B |
| 201 | 137 | 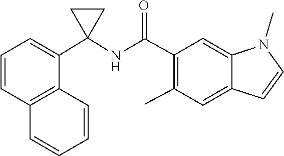 [SMILES: Cc1cc2ccn(C)c2cc1C(=O)NC1(c2cccc3ccccc23)CC1] | B |
| 296 | 138 | 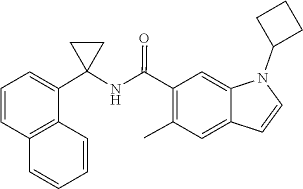 [SMILES: Cc1cc2ccn(C3CCC3)c2cc1C(=O)NC1(c2cccc3ccccc23)CC1] | B |
| 218 | 139 | 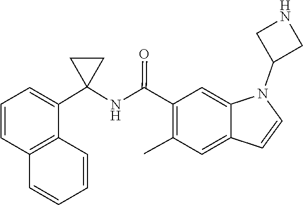 [SMILES: Cc1cc2ccn(C3CNC3)c2cc1C(=O)NC1(c2cccc3ccccc23)CC1] | A |
| 290 | 140 | 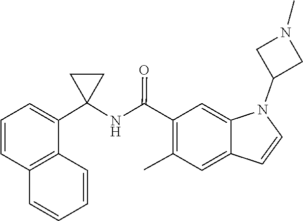 [SMILES: Cc1cc2ccn(C3CN(C)C3)c2cc1C(=O)NC1(c2cccc3ccccc23)CC1] | A |
| 239 | 141 | 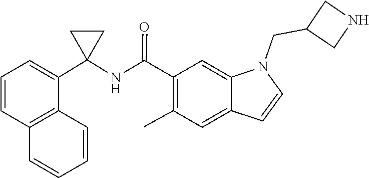 [SMILES: Cc1cc2ccn(CC3CNC3)c2cc1C(=O)NC1(c2cccc3ccccc23)CC1] | A |
| 295 | 142 | 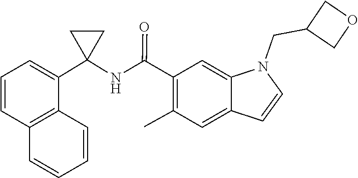 [SMILES: Cc1cc2ccn(CC3COC3)c2cc1C(=O)NC1(c2cccc3ccccc23)CC1] | B |
| 203 | 143 | 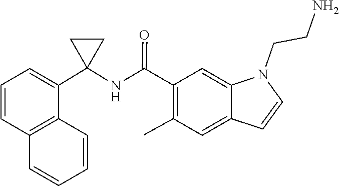 [SMILES: Cc1cc2ccn(CCN)c2cc1C(=O)NC1(c2cccc3ccccc23)CC1] | B |
| 212 | 144 | 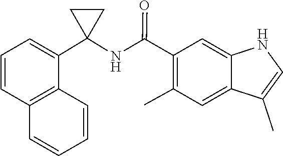 [SMILES: Cc1cc2c(C)c[nH]c2cc1C(=O)NC1(c2cccc3ccccc23)CC1] | B |
| 292 | 145 | 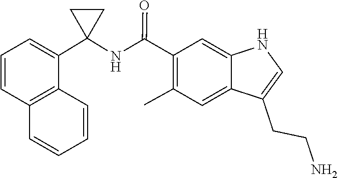 [SMILES: Cc1cc2c(CCN)c[nH]c2cc1C(=O)NC1(c2cccc3ccccc23)CC1] | B |
| 207 | 146 | 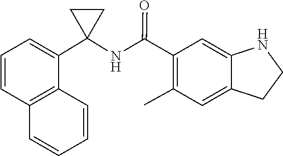 [SMILES: Cc1cc2c(cc1C(=O)NC1(c3cccc4ccccc34)CC1)NCC2] | B |
| 210 | 147 | 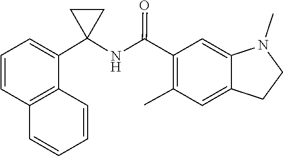 [SMILES: Cc1cc2c(cc1C(=O)NC1(c3cccc4ccccc34)CC1)N(C)CC2] | B |
| 249 | 148 | 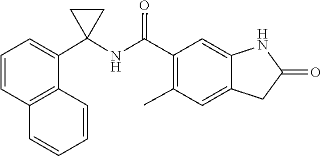 [SMILES: Cc1cc2c(cc1C(=O)NC1(c3cccc4ccccc34)CC1)NC(=O)C2] | B |
| 243 | 149 | 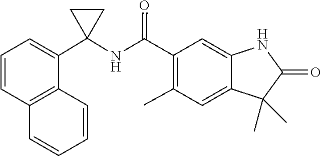 [SMILES: Cc1cc2c(cc1C(=O)NC1(c3cccc4ccccc34)CC1)NC(=O)C2(C)C] | C |
| 262 | 150 | 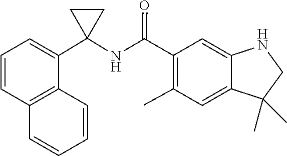 [SMILES: Cc1cc2c(cc1C(=O)NC1(c3cccc4ccccc34)CC1)NCC2(C)C] | B |
| 190 | 151 | 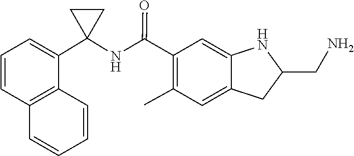 [SMILES: Cc1cc2c(cc1C(=O)NC1(c3cccc4ccccc34)CC1)NC(CN)C2] | A |
| 231 | 152 | 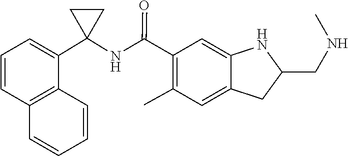 [SMILES: CNCC1Cc2cc(C)c(C(=O)NC3(c4cccc5ccccc45)CC3)cc2N1] | A |
| 176 | 153 | 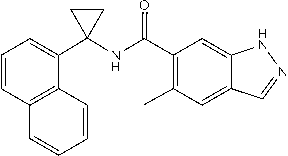 [SMILES: Cc1cc2cn[nH]c2cc1C(=O)NC1(c2cccc3ccccc23)CC1] | B |
| 304 | 154 | 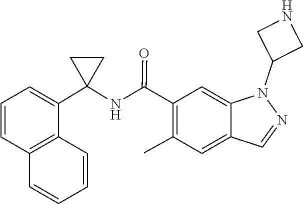 [SMILES: Cc1cc2cnn(C3CNC3)c2cc1C(=O)NC1(c2cccc3ccccc23)CC1] | A |
| 305 | 154 | 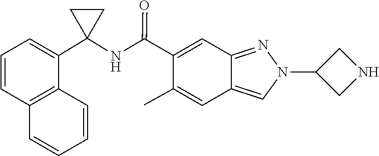 [SMILES: Cc1cc2cn(C3CNC3)nc2cc1C(=O)NC1(c2cccc3ccccc23)CC1] | B |
| 197 | 155 | 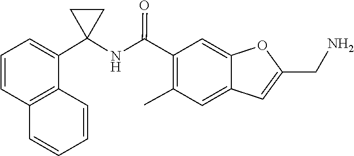 [SMILES: Cc1cc2cc(CN)oc2cc1C(=O)NC1(c2cccc3ccccc23)CC1] | A |
| 198 | 156 | 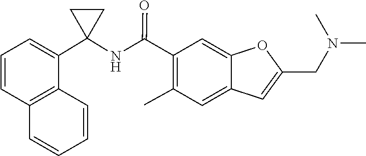 [SMILES: Cc1cc2cc(CN(C)C)oc2cc1C(=O)NC1(c2cccc3ccccc23)CC1] | B |
| 220 | 157 | 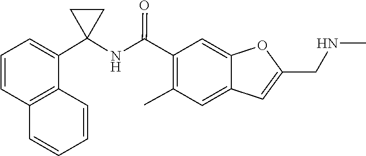 [SMILES: CNCc1cc2cc(C)c(C(=O)NC3(c4cccc5ccccc45)CC3)cc2o1] | A |
| 235 | 158 | 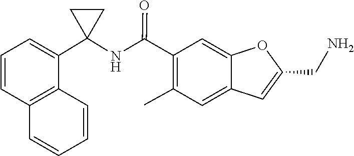 | B |
| 236 | 158 | 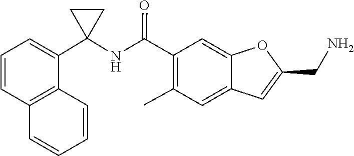 | B |
| 230 | 159 | 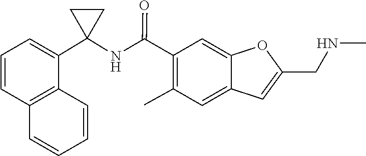 [SMILES: CNCc1cc2cc(C)c(C(=O)NC3(c4cccc5ccccc45)CC3)cc2o1] | B |
| 224 | 160 | 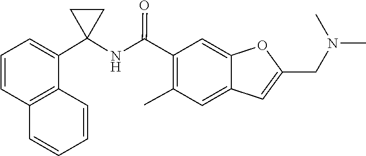 [SMILES: Cc1cc2cc(CN(C)C)oc2cc1C(=O)NC1(c2cccc3ccccc23)CC1] | B |
| 397 | 161 | 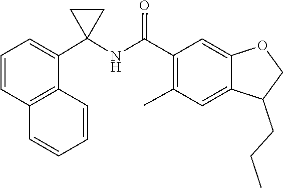 [SMILES: CCCC1COc2cc(C(=O)NC3(c4cccc5ccccc45)CC3)c(C)cc21] | B |
| 223 | 162 | 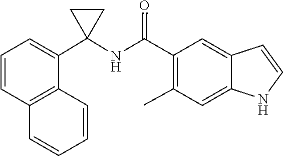 [SMILES: Cc1cc2[nH]ccc2cc1C(=O)NC1(c2cccc3ccccc23)CC1] | B |
| 280 | 163 | 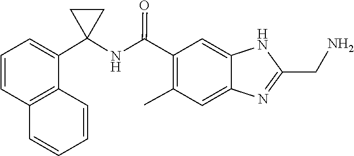 [SMILES: Cc1cc2nc(CN)[nH]c2cc1C(=O)NC1(c2cccc3ccccc23)CC1] | B |
| 245 | 164 | 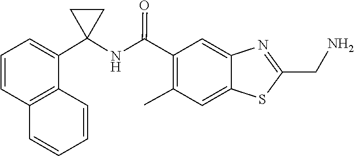 [SMILES: Cc1cc2sc(CN)nc2cc1C(=O)NC1(c2cccc3ccccc23)CC1] | A |
| 238 | 165 | 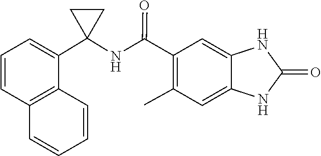 [SMILES: Cc1cc2[nH]c(=O)[nH]c2cc1C(=O)NC1(c2cccc3ccccc23)CC1] | B |
| 237 | 166 | 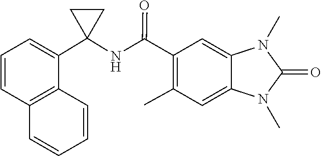 [SMILES: Cc1cc2c(cc1C(=O)NC1(c3cccc4ccccc34)CC1)n(C)c(=O)n2C] | C |
| 297 | 167 | 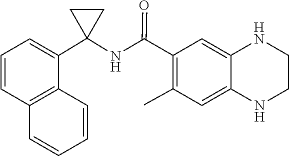 [SMILES: Cc1cc2c(cc1C(=O)NC1(c3cccc4ccccc34)CC1)NCCN2] | B |
| 227 | 168 | 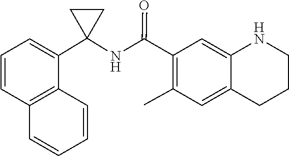 [SMILES: Cc1cc2c(cc1C(=O)NC1(c3cccc4ccccc34)CC1)NCCC2] | B |
| 334 | 169 | 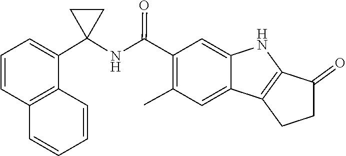 [SMILES: Cc1cc2c3c([nH]c2cc1C(=O)NC1(c2cccc4ccccc24)CC1)C(=O)CC3] | C |
| 194 | 170 | 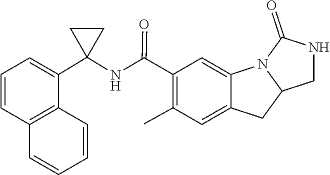 [SMILES: Cc1cc2c(cc1C(=O)NC1(c3cccc4ccccc34)CC1)N1C(=O)NCC1C2] | C |
| 272 | 171 | 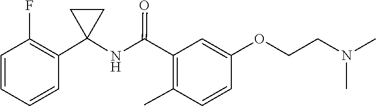 [SMILES: Cc1ccc(OCCN(C)C)cc1C(=O)NC1(c2ccccc2F)CC1] | D |
| 252 | 172 | 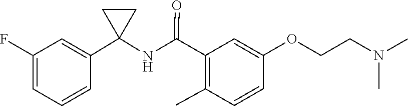 [SMILES: Cc1ccc(OCCN(C)C)cc1C(=O)NC1(c2cccc(F)c2)CC1] | D |
| 259 | 173 | 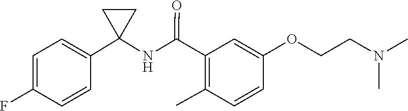 [SMILES: Cc1ccc(OCCN(C)C)cc1C(=O)NC1(c2ccc(F)cc2)CC1] | D |
| 251 | 174 | 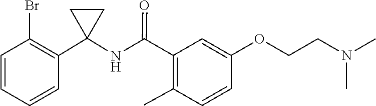 [SMILES: Cc1ccc(OCCN(C)C)cc1C(=O)NC1(c2ccccc2Br)CC1] | B |
| 261 | 175 | 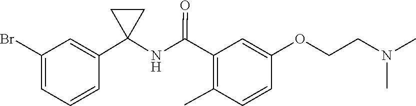 [SMILES: Cc1ccc(OCCN(C)C)cc1C(=O)NC1(c2cccc(Br)c2)CC1] | C |
| 258 | 176 | 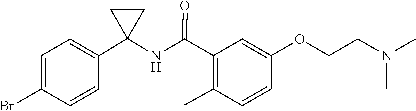 [SMILES: Cc1ccc(OCCN(C)C)cc1C(=O)NC1(c2ccc(Br)cc2)CC1] | D |
| 326 | 177 | 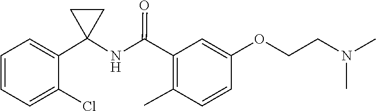 [SMILES: Cc1ccc(OCCN(C)C)cc1C(=O)NC1(c2ccccc2Cl)CC1] | C |
| 282 | 178 | 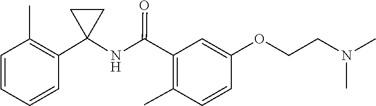 [SMILES: Cc1ccc(OCCN(C)C)cc1C(=O)NC1(c2ccccc2C)CC1] | C |
| 255 | 179 | 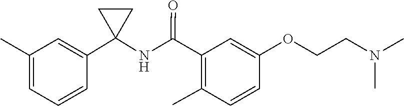 | D |
| 253 | 180 | 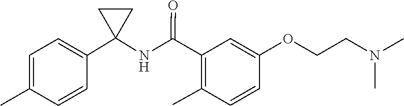 | D |
| 321 | 181 | 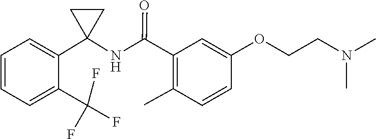 [SMILES: Cc1ccc(OCCN(C)C)cc1C(=O)NC1(c2ccccc2C(F)(F)F)CC1] | B |
| 364 | 182 | 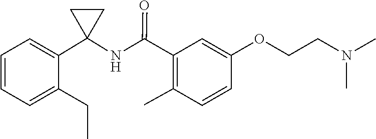 [SMILES: CCc1ccccc1C1(NC(=O)c2cc(OCCN(C)C)ccc2C)CC1] | B |
| 344 | 183 | 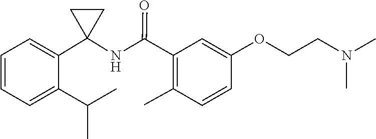 [SMILES: Cc1ccc(OCCN(C)C)cc1C(=O)NC1(c2ccccc2C(C)C)CC1] | B |
| 267 | 184 | 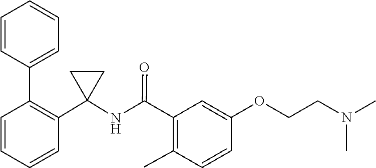 [SMILES: Cc1ccc(OCCN(C)C)cc1C(=O)NC1(c2ccccc2-c2ccccc2)CC1] | D |
| 294 | 185 | 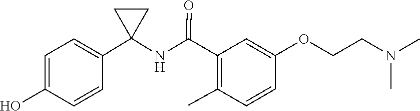 [SMILES: Cc1ccc(OCCN(C)C)cc1C(=O)NC1(c2ccc(O)cc2)CC1] | D |
| 271 | 186 | 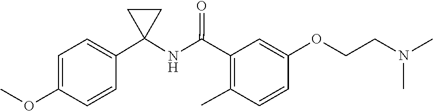 | D |
| 298 | 187 | 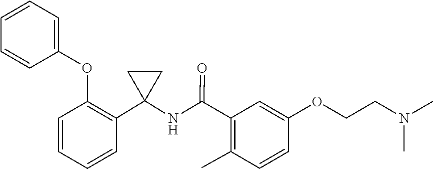 [SMILES: Cc1ccc(OCCN(C)C)cc1C(=O)NC1(c2ccccc2Oc2ccccc2)CC1] | C |
| 270 | 188 | 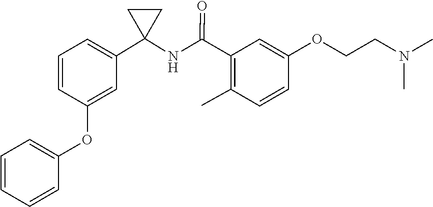 [SMILES: Cc1ccc(OCCN(C)C)cc1C(=O)NC1(c2cccc(Oc3ccccc3)c2)CC1] | D |
| 279 | 189 | 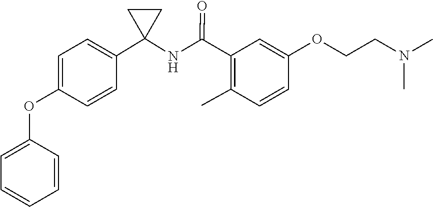 [SMILES: Cc1ccc(OCCN(C)C)cc1C(=O)NC1(c2ccc(Oc3ccccc3)cc2)CC1] | C |
| 327 | 190 | 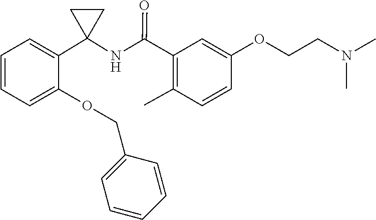 [SMILES: Cc1ccc(OCCN(C)C)cc1C(=O)NC1(c2ccccc2OCc2ccccc2)CC1] | C |
| 308 | 191 | 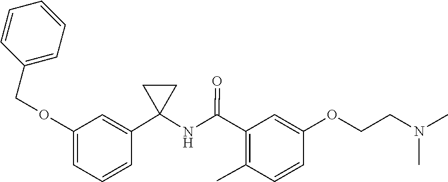 [SMILES: Cc1ccc(OCCN(C)C)cc1C(=O)NC1(c2cccc(OCc3ccccc3)c2)CC1] | D |
| 307 | 192 | 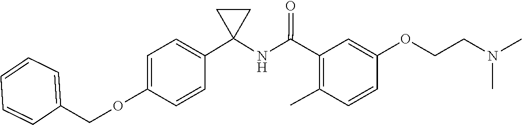 [SMILES: Cc1ccc(OCCN(C)C)cc1C(=O)NC1(c2ccc(OCc3ccccc3)cc2)CC1] | D |
| 291 | 193 | 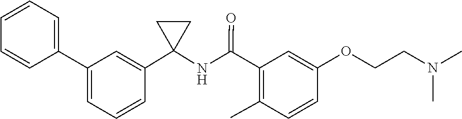 [SMILES: Cc1ccc(OCCN(C)C)cc1C(=O)NC1(c2cccc(-c3ccccc3)c2)CC1] | B |
| 264 | 194 | 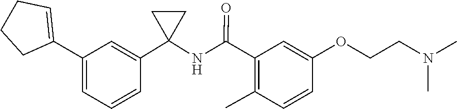 [SMILES: Cc1ccc(OCCN(C)C)cc1C(=O)NC1(c2cccc(C3=CCCC3)c2)CC1] | C |
| 276 | 195 | 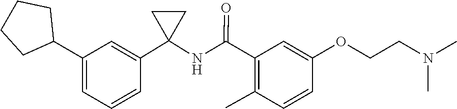 [SMILES: Cc1ccc(OCCN(C)C)cc1C(=O)NC1(c2cccc(C3CCCC3)c2)CC1] | D |
| 338 | 196 | 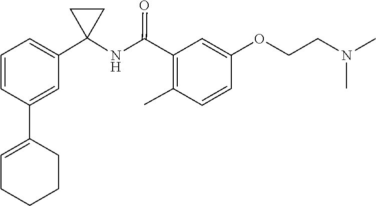 [SMILES: Cc1ccc(OCCN(C)C)cc1C(=O)NC1(c2cccc(C3=CCCCC3)c2)CC1] | C |
| 266 | 197 | 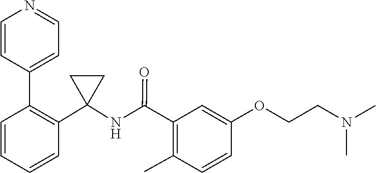 [SMILES: Cc1ccc(OCCN(C)C)cc1C(=O)NC1(c2ccccc2-c2ccncc2)CC1] | D |
| 355 | 198 | 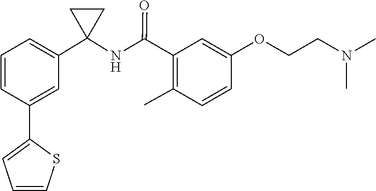 [SMILES: Cc1ccc(OCCN(C)C)cc1C(=O)NC1(c2cccc(-c3cccs3)c2)CC1] | B |
| 325 | 199 | 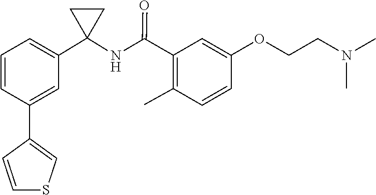 [SMILES: Cc1ccc(OCCN(C)C)cc1C(=O)NC1(c2cccc(-c3ccsc3)c2)CC1] | B |
| 169 | 200 | 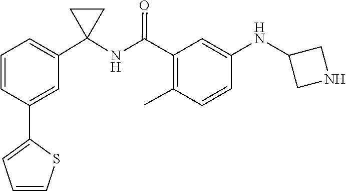 [SMILES: Cc1ccc(NC2CNC2)cc1C(=O)NC1(c2cccc(-c3cccs3)c2)CC1] | B |
| 187 | 201 | 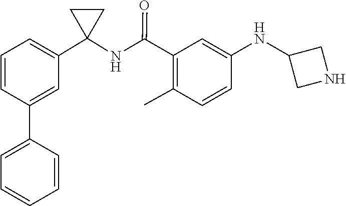 [SMILES: Cc1ccc(NC2CNC2)cc1C(=O)NC1(c2cccc(-c3ccccc3)c2)CC1] | C |
| 186 | 202 | 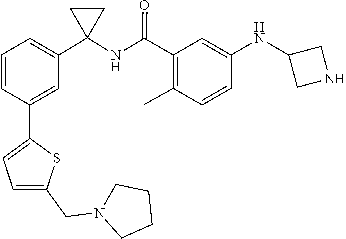 [SMILES: Cc1ccc(NC2CNC2)cc1C(=O)NC1(c2cccc(-c3ccc(CN4CCCC4)s3)c2)CC1] | B |
| 196 | 203 | 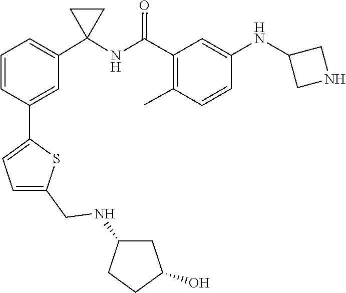 [SMILES: Cc1ccc(NC2CNC2)cc1C(=O)NC1(c2cccc(-c3ccc(CN[C@H]4CC[C@@H](O)C4)s3)c2)CC1] | B |
| 318 | 204 | 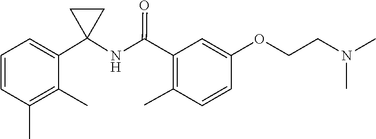 [SMILES: Cc1ccc(OCCN(C)C)cc1C(=O)NC1(c2cccc(C)c2C)CC1] | C |
| 335 | 205 | 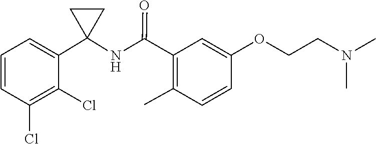 [SMILES: Cc1ccc(OCCN(C)C)cc1C(=O)NC1(c2cccc(Cl)c2Cl)CC1] | B |
| 317 | 206 | 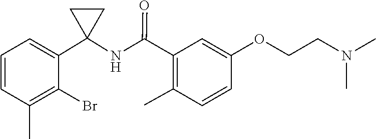 [SMILES: Cc1ccc(OCCN(C)C)cc1C(=O)NC1(c2cccc(C)c2Br)CC1] | B |
| 336 | 207 | 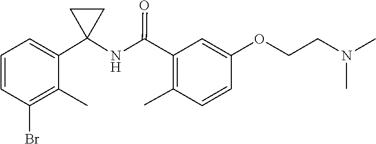 [SMILES: Cc1ccc(OCCN(C)C)cc1C(=O)NC1(c2cccc(Br)c2C)CC1] | B |
| 340 | 208 | 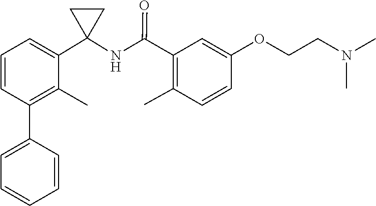 [SMILES: Cc1ccc(OCCN(C)C)cc1C(=O)NC1(c2cccc(-c3ccccc3)c2C)CC1] | B |
| 329 | 209 | 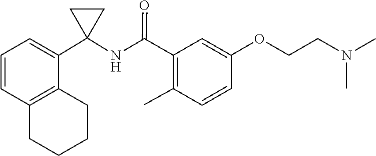 [SMILES: Cc1ccc(OCCN(C)C)cc1C(=O)NC1(c2cccc3c2CCCC3)CC1] | B |
| 370 | 210 | 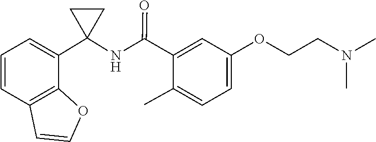 [SMILES: Cc1ccc(OCCN(C)C)cc1C(=O)NC1(c2cccc3ccoc23)CC1] | B |
| 357 | 211 | 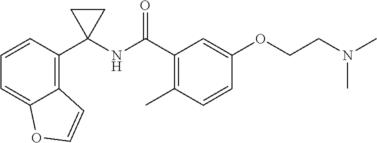 [SMILES: Cc1ccc(OCCN(C)C)cc1C(=O)NC1(c2cccc3occc23)CC1] | B |
| 368 | 212 | 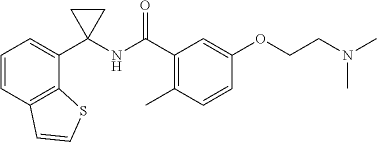 [SMILES: Cc1ccc(OCCN(C)C)cc1C(=O)NC1(c2cccc3ccsc23)CC1] | B |
| 356 | 213 | 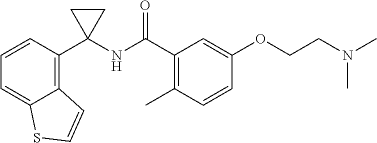 [SMILES: Cc1ccc(OCCN(C)C)cc1C(=O)NC1(c2cccc3sccc23)CC1] | A |
| 403 | 214 | 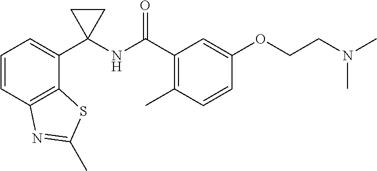 [SMILES: Cc1nc2cccc(C3(NC(=O)c4cc(OCCN(C)C)ccc4C)CC3)c2s1] | B |
| 346 | 215 | 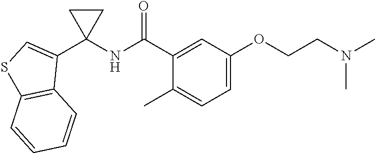 [SMILES: Cc1ccc(OCCN(C)C)cc1C(=O)NC1(c2csc3ccccc23)CC1] | B |
| 354 | 216 | 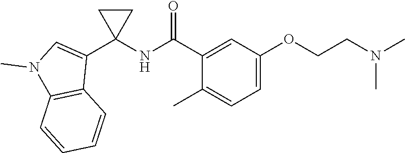 | B |
| 395 | 217 | 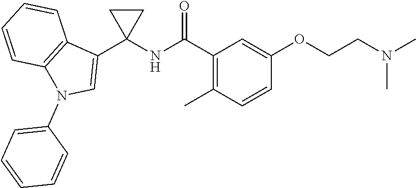 [SMILES: Cc1ccc(OCCN(C)C)cc1C(=O)NC1(c2cn(-c3ccccc3)c3ccccc23)CC1] | A |
| 301 | 218 | 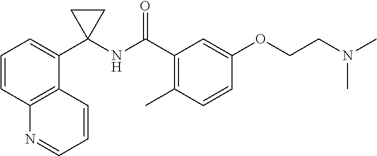 [SMILES: Cc1ccc(OCCN(C)C)cc1C(=O)NC1(c2cccc3ncccc23)CC1] | A |
| 374 | 219 | 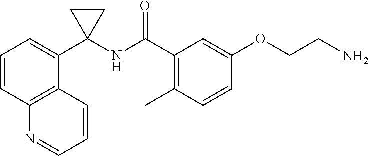 [SMILES: Cc1ccc(OCCN)cc1C(=O)NC1(c2cccc3ncccc23)CC1] | A |
| 385 | 220 | 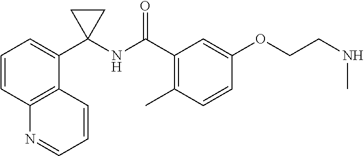 [SMILES: CNCCOc1ccc(C)c(C(=O)NC2(c3cccc4ncccc34)CC2)c1] | A |
| 381 | 221 | 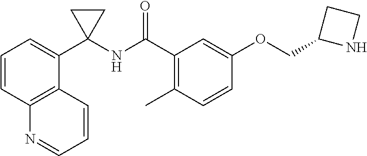 [SMILES: Cc1ccc(OC[C@@H]2CCN2)cc1C(=O)NC1(c2cccc3ncccc23)CC1] | A |
| 383 | 222 | 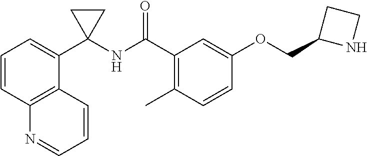 [SMILES: Cc1ccc(OC[C@H]2CCN2)cc1C(=O)NC1(c2cccc3ncccc23)CC1] | A |
| 391 | 223 | 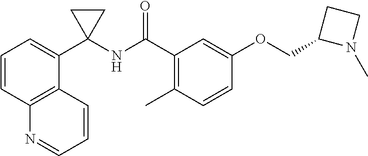 [SMILES: Cc1ccc(OC[C@@H]2CCN2C)cc1C(=O)NC1(c2cccc3ncccc23)CC1] | A |
| 390 | 224 | 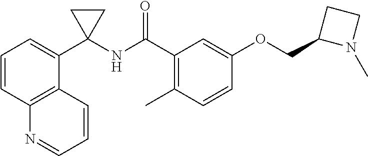 [SMILES: Cc1ccc(OC[C@H]2CCN2C)cc1C(=O)NC1(c2cccc3ncccc23)CC1] | A |
| 380 | 225 | 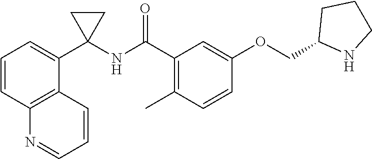 [SMILES: Cc1ccc(OC[C@@H]2CCCN2)cc1C(=O)NC1(c2cccc3ncccc23)CC1] | A |
| 379 | 226 | 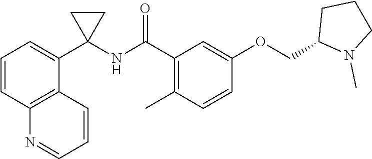 [SMILES: Cc1ccc(OC[C@@H]2CCCN2C)cc1C(=O)NC1(c2cccc3ncccc23)CC1] | A |
| 371 | 227 | 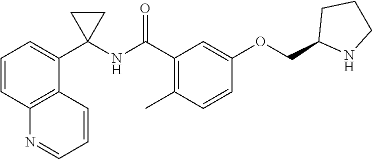 [SMILES: Cc1ccc(OC[C@H]2CCCN2)cc1C(=O)NC1(c2cccc3ncccc23)CC1] | A |
| 382 | 228 | 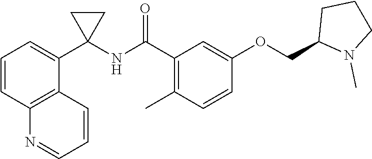 [SMILES: Cc1ccc(OC[C@H]2CCCN2C)cc1C(=O)NC1(c2cccc3ncccc23)CC1] | A |
| 299 | 229 | 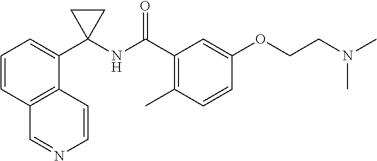 [SMILES: Cc1ccc(OCCN(C)C)cc1C(=O)NC1(c2cccc3cnccc23)CC1] | A |
| 375 | 230 | 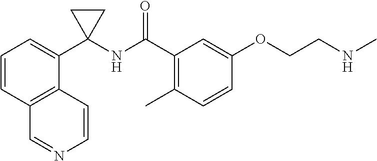 [SMILES: CNCCOc1ccc(C)c(C(=O)NC2(c3cccc4cnccc34)CC2)c1] | A |
| 402 | 231 | 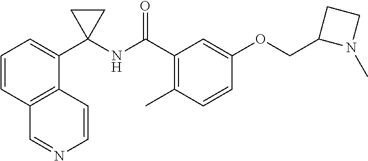 [SMILES: Cc1ccc(OCC2CCN2C)cc1C(=O)NC1(c2cccc3cnccc23)CC1] | A |
| 333 | 232 | 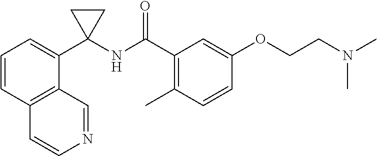 [SMILES: Cc1ccc(OCCN(C)C)cc1C(=O)NC1(c2cccc3ccncc23)CC1] | B |
| 388 | 233 | 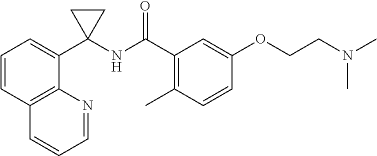 [SMILES: Cc1ccc(OCCN(C)C)cc1C(=O)NC1(c2cccc3cccnc23)CC1] | A |
| 363 | 234 | 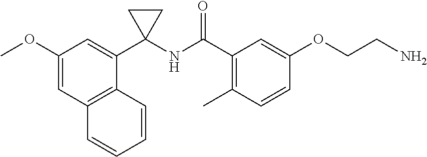 [SMILES: COc1cc(C2(NC(=O)c3cc(OCCN)ccc3C)CC2)c2ccccc2c1] | A |
| 320 | 235 | 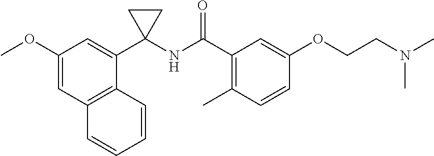 | A |
| 345 | 236 | 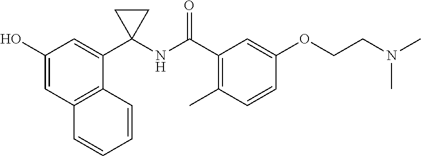 [SMILES: Cc1ccc(OCCN(C)C)cc1C(=O)NC1(c2cc(O)cc3ccccc23)CC1] | A |
| 347 | 237 | 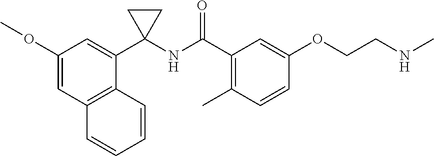 | A |
| 384 | 238 | 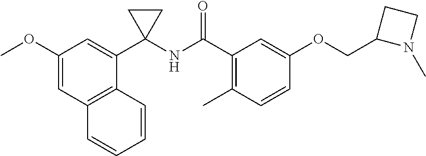 | A |
| 401 | 239 | 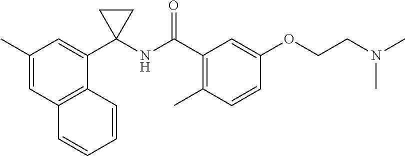 | A |
| 319 | 240 | 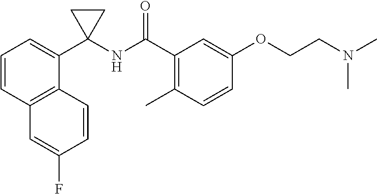 [SMILES: Cc1ccc(OCCN(C)C)cc1C(=O)NC1(c2cccc3cc(F)ccc23)CC1] | A |
| 392 | 241 | 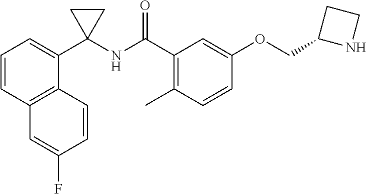 [SMILES: Cc1ccc(OC[C@@H]2CCN2)cc1C(=O)NC1(c2cccc3cc(F)ccc23)CC1] | A |
| 377 | 242 | 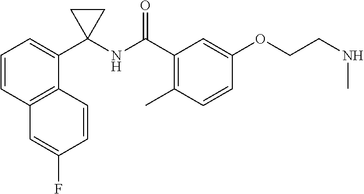 [SMILES: CNCCOc1ccc(C)c(C(=O)NC2(c3cccc4cc(F)ccc34)CC2)c1] | A |
| 389 | 243 | 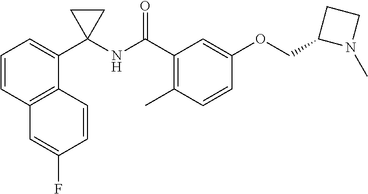 [SMILES: Cc1ccc(OC[C@@H]2CCN2C)cc1C(=O)NC1(c2cccc3cc(F)ccc23)CC1] | A |
| 365 | 244 | 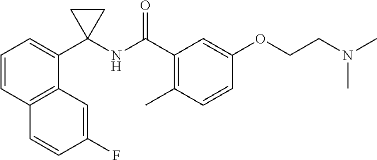 [SMILES: Cc1ccc(OCCN(C)C)cc1C(=O)NC1(c2cccc3ccc(F)cc23)CC1] | A |
| 120 | 245 | 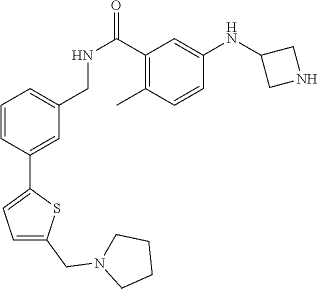 [SMILES: Cc1ccc(NC2CNC2)cc1C(=O)NCc1cccc(-c2ccc(CN3CCCC3)s2)c1] | D |
| 126 | 246 | 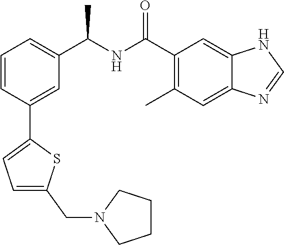 [SMILES: Cc1cc2nc[nH]c2cc1C(=O)N[C@H](C)c1cccc(-c2ccc(CN3CCCC3)s2)c1] | C |
| 134 | 247 | 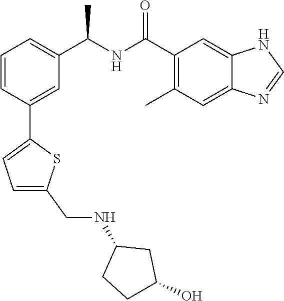 [SMILES: Cc1cc2nc[nH]c2cc1C(=O)N[C@H](C)c1cccc(-c2ccc(CN[C@H]3CC[C@@H](O)C3)s2)c1] | B |
| 137 | 248 | 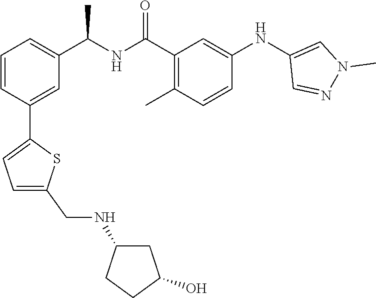 [SMILES: Cc1ccc(Nc2cnn(C)c2)cc1C(=O)N[C@H](C)c1cccc(-c2ccc(CN[C@H]3CC[C@@H](O)C3)s2)c1] | C |
| 140 | 249 | 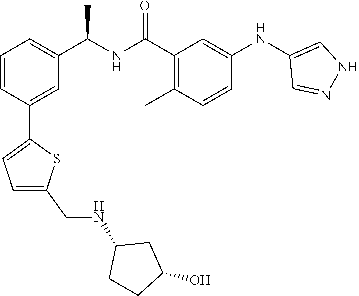 [SMILES: Cc1ccc(Nc2cn[nH]c2)cc1C(=O)N[C@H](C)c1cccc(-c2ccc(CN[C@H]3CC[C@@H](O)C3)s2)c1] | C |
| 146 | 250 | 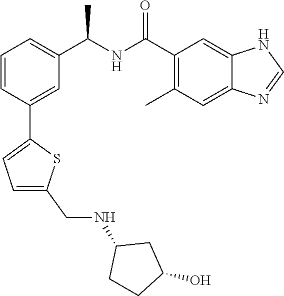 [SMILES: Cc1cc2nc[nH]c2cc1C(=O)N[C@H](C)c1cccc(-c2ccc(CN[C@H]3CC[C@@H](O)C3)s2)c1] | B |
| 151 | 251 | 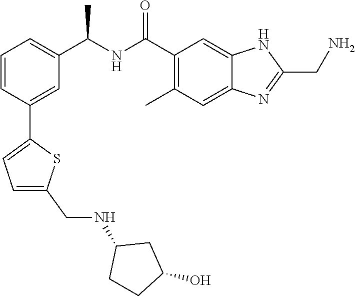 [SMILES: Cc1cc2nc(CN)[nH]c2cc1C(=O)N[C@H](C)c1cccc(-c2ccc(CN[C@H]3CC[C@@H](O)C3)s2)c1] | B |
| 154 | 252 | 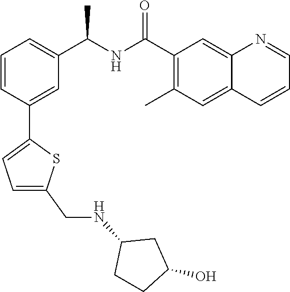 [SMILES: Cc1cc2cccnc2cc1C(=O)N[C@H](C)c1cccc(-c2ccc(CN[C@H]3CC[C@@H](O)C3)s2)c1] | C |
| 143 | 253 | 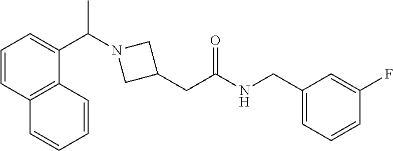 [SMILES: CC(c1cccc2ccccc12)N1CC(CC(=O)NCc2cccc(F)c2)C1] | D |
| 144 | 254 | 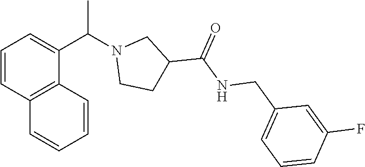 [SMILES: CC(c1cccc2ccccc12)N1CCC(C(=O)NCc2cccc(F)c2)C1] | D |
| 149 | 255 | 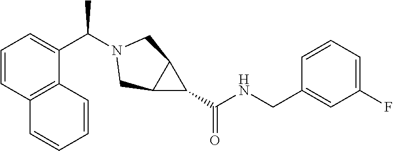 [SMILES: C[C@H](c1cccc2ccccc12)N1C[C@@H]2[C@H](C1)[C@H]2C(=O)NCc1cccc(F)c1] | D |
| 135 | 256 | 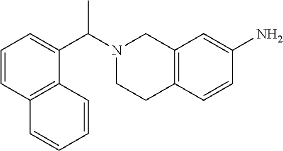 [SMILES: CC(c1cccc2ccccc12)N1CCc2ccc(N)cc2C1] | C |
| 139 | 257 | 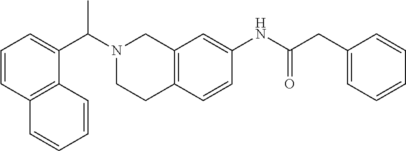 [SMILES: CC(c1cccc2ccccc12)N1CCc2ccc(NC(=O)Cc3ccccc3)cc2C1] | D |
| 404 | 258 | 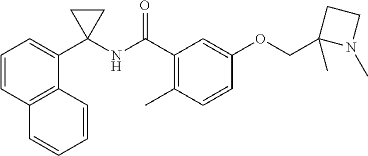 [SMILES: Cc1ccc(OCC2(C)CCN2C)cc1C(=O)NC1(c2cccc3ccccc23)CC1] | A |
| 406 | 259 | 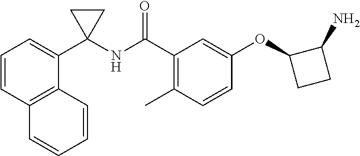 [SMILES: Cc1ccc(O[C@@H]2CC[C@@H]2N)cc1C(=O)NC1(c2cccc3ccccc23)CC1] | A |
| 416 | 260 | 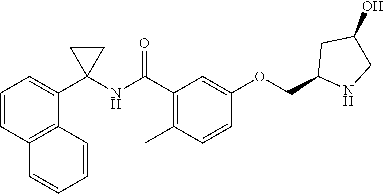 [SMILES: Cc1ccc(OC[C@H]2C[C@@H](O)CN2)cc1C(=O)NC1(c2cccc3ccccc23)CC1] | A |
| 414 | 261 | 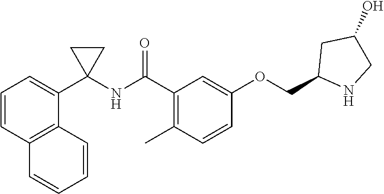 [SMILES: Cc1ccc(OC[C@H]2C[C@H](O)CN2)cc1C(=O)NC1(c2cccc3ccccc23)CC1] | A |
| 412 | 262 | 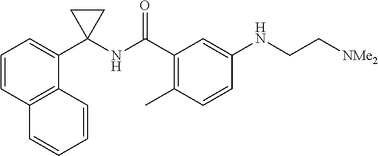 [SMILES: Cc1ccc(NCCN(C)C)cc1C(=O)NC1(c2cccc3ccccc23)CC1] | A |
| 421 | 263 | 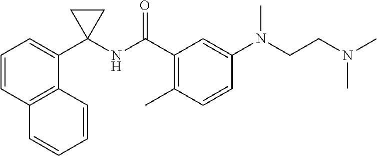 [SMILES: Cc1ccc(N(C)CCN(C)C)cc1C(=O)NC1(c2cccc3ccccc23)CC1] | A |
| 413 | 264 | 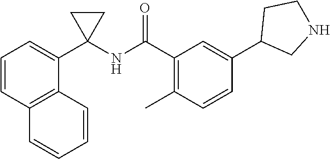 [SMILES: Cc1ccc(C2CCNC2)cc1C(=O)NC1(c2cccc3ccccc23)CC1] | A |
| 407 | 265 | 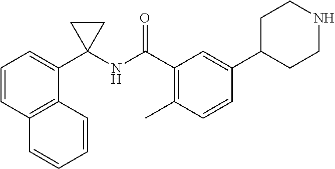 [SMILES: Cc1ccc(C2CCNCC2)cc1C(=O)NC1(c2cccc3ccccc23)CC1] | A |
| 410 | 266 | 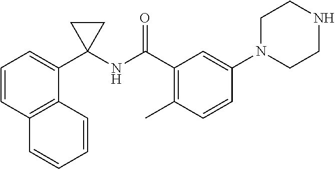 [SMILES: Cc1ccc(N2CCNCC2)cc1C(=O)NC1(c2cccc3ccccc23)CC1] | A |
| 418 | 267 | 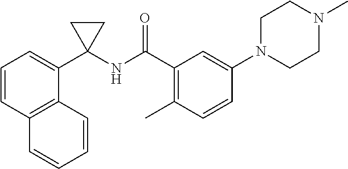 [SMILES: Cc1ccc(N2CCN(C)CC2)cc1C(=O)NC1(c2cccc3ccccc23)CC1] | A |
| 411 | 268 | 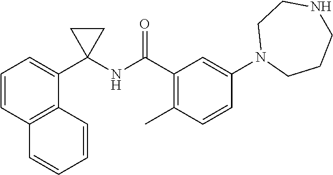 [SMILES: Cc1ccc(N2CCCNCC2)cc1C(=O)NC1(c2cccc3ccccc23)CC1] | A |
| 405 | 269 | 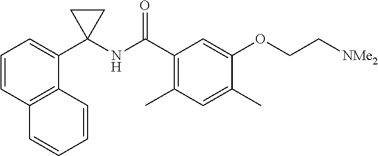 [SMILES: Cc1cc(C)c(C(=O)NC2(c3cccc4ccccc34)CC2)cc1OCCN(C)C] | A |
| 409 | 270 | 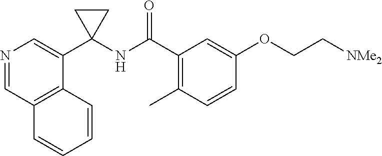 [SMILES: Cc1ccc(OCCN(C)C)cc1C(=O)NC1(c2cncc3ccccc23)CC1] | A |
| 415 | 271 | 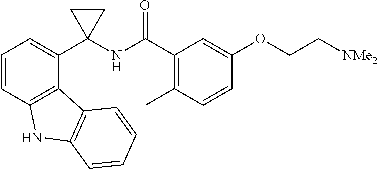 [SMILES: Cc1ccc(OCCN(C)C)cc1C(=O)NC1(c2cccc3[nH]c4ccccc4c23)CC1] | B |
| 417 | 272 | 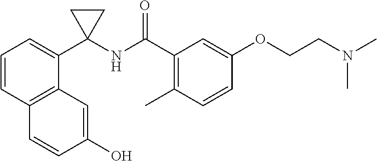 [SMILES: Cc1ccc(OCCN(C)C)cc1C(=O)NC1(c2cccc3ccc(O)cc23)CC1] | A |
| 408 | 273 | 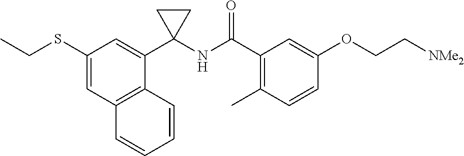 [SMILES: CCSc1cc(C2(NC(=O)c3cc(OCCN(C)C)ccc3C)CC2)c2ccccc2c1] | A |
| 419 | 274 | 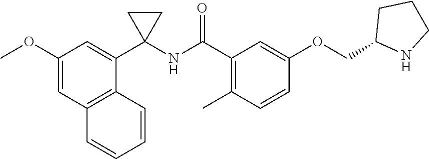 [SMILES: COc1cc(C2(NC(=O)c3cc(OC[C@@H]4CCCN4)ccc3C)CC2)c2ccccc2c1] | A |
| 420 | 275 | 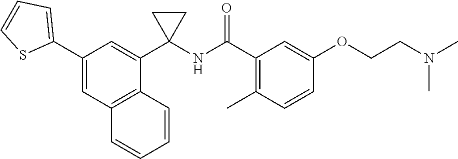 [SMILES: Cc1ccc(OCCN(C)C)cc1C(=O)NC1(c2cc(-c3cccs3)cc3ccccc23)CC1] | A |
| 459 | 276 | 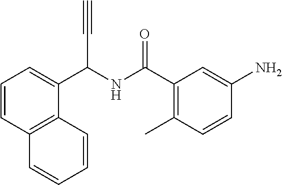 [SMILES: C#CC(NC(=O)c1cc(N)ccc1C)c1cccc2ccccc12] | D |
| 448 | 277 | 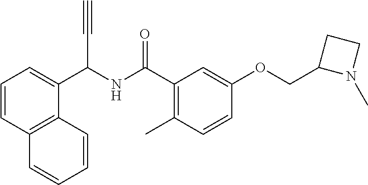 [SMILES: C#CC(NC(=O)c1cc(OCC2CCN2C)ccc1C)c1cccc2ccccc12] | C |
| 453 | 278 | 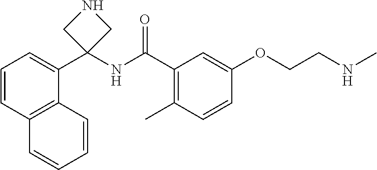 [SMILES: CNCCOc1ccc(C)c(C(=O)NC2(c3cccc4ccccc34)CNC2)c1] | D |
| 499 | 279 | 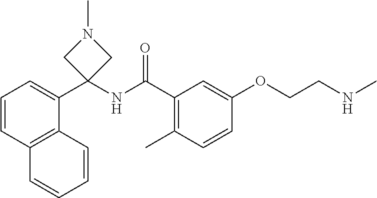 [SMILES: CNCCOc1ccc(C)c(C(=O)NC2(c3cccc4ccccc34)CN(C)C2)c1] | D |
| 451 | 280 | 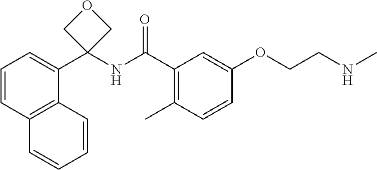 [SMILES: CNCCOc1ccc(C)c(C(=O)NC2(c3cccc4ccccc34)COC2)c1] | A |
| 450 | 281 | 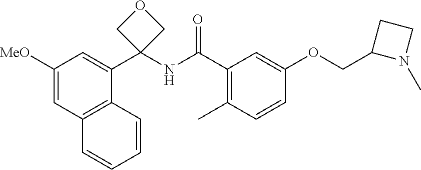 [SMILES: COc1cc(C2(NC(=O)c3cc(OCC4CCN4C)ccc3C)COC2)c2ccccc2c1] | A |
| 425 | 282 | 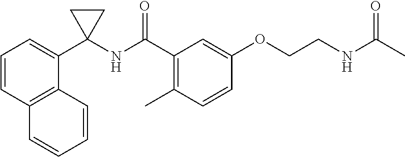 [SMILES: CC(=O)NCCOc1ccc(C)c(C(=O)NC2(c3cccc4ccccc34)CC2)c1] | B |
| 424 | 283 | 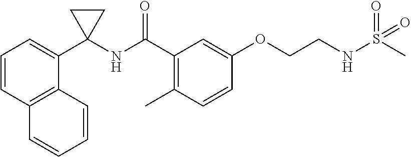 [SMILES: Cc1ccc(OCCNS(C)(=O)=O)cc1C(=O)NC1(c2cccc3ccccc23)CC1] | B |
| 439 | 284 | 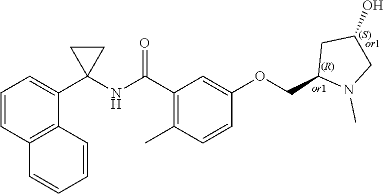 | A |
| 440 | 285 | 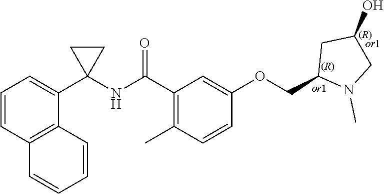 | A |
| 438 | 286 | 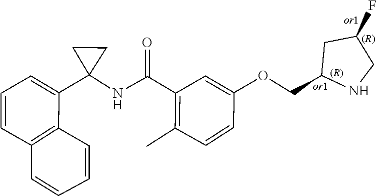 | A |
| 437 | 287 | 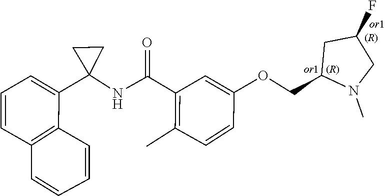 | A |
| 442 | 288 | 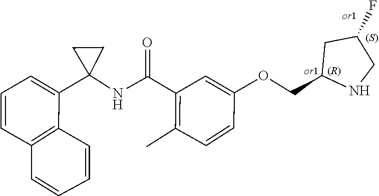 | A |
| 441 | 289 | 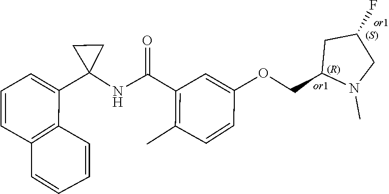 | A |
| 491 | 290 | 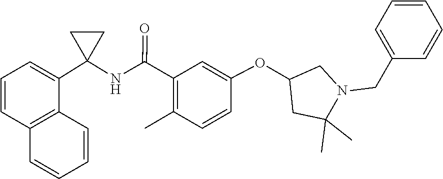 [SMILES: Cc1ccc(OC2CN(Cc3ccccc3)C(C)(C)C2)cc1C(=O)NC1(c2cccc3ccccc23)CC1] | B |
| 492 | 290 | 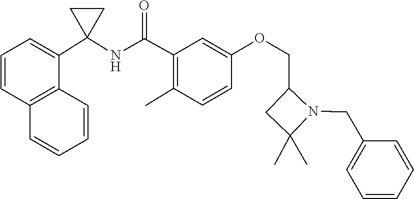 [SMILES: Cc1ccc(OCC2CC(C)(C)N2Cc2ccccc2)cc1C(=O)NC1(c2cccc3ccccc23)CC1] | B |
| 534 | 291 | 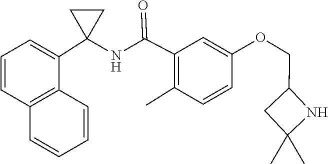 [SMILES: Cc1ccc(OCC2CC(C)(C)N2)cc1C(=O)NC1(c2cccc3ccccc23)CC1] | A |
| 533 | 292 | 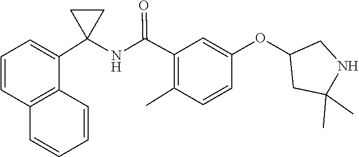 [SMILES: Cc1ccc(OC2CNC(C)(C)C2)cc1C(=O)NC1(c2cccc3ccccc23)CC1] | A |
| 487 | 293 | 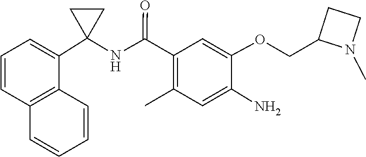 [SMILES: Cc1cc(N)c(OCC2CCN2C)cc1C(=O)NC1(c2cccc3ccccc23)CC1] | A |
| 435 | 294 | 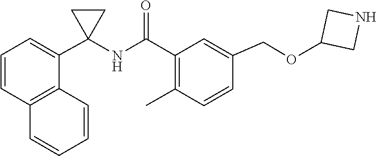 [SMILES: Cc1ccc(COC2CNC2)cc1C(=O)NC1(c2cccc3ccccc23)CC1] | B |
| 447 | 295 | 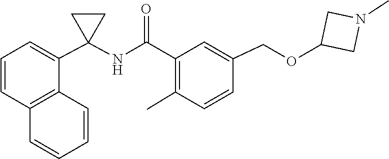 [SMILES: Cc1ccc(COC2CN(C)C2)cc1C(=O)NC1(c2cccc3ccccc23)CC1] | B |
| 449 | 296 | 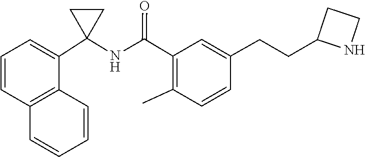 [SMILES: Cc1ccc(CCC2CCN2)cc1C(=O)NC1(c2cccc3ccccc23)CC1] | A |
| 455 | 297 | 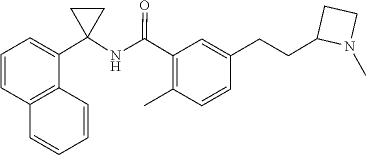 [SMILES: Cc1ccc(CCC2CCN2C)cc1C(=O)NC1(c2cccc3ccccc23)CC1] | A |
| 431 | 298 | 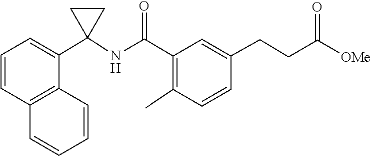 [SMILES: COC(=O)CCc1ccc(C)c(C(=O)NC2(c3cccc4ccccc34)CC2)c1] | B |
| 452 | 299 | 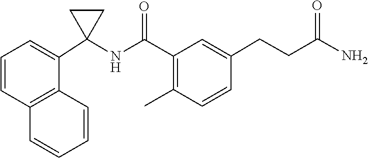 [SMILES: Cc1ccc(CCC(N)=O)cc1C(=O)NC1(c2cccc3ccccc23)CC1] | B |
| 472 | 300 | 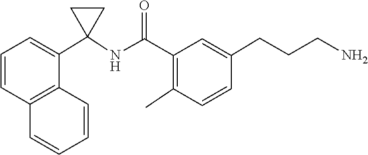 [SMILES: Cc1ccc(CCCN)cc1C(=O)NC1(c2cccc3ccccc23)CC1] | A |
| 463 | 301 | 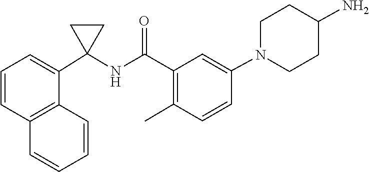 [SMILES: Cc1ccc(N2CCC(N)CC2)cc1C(=O)NC1(c2cccc3ccccc23)CC1] | B |
| 505 | 302 | 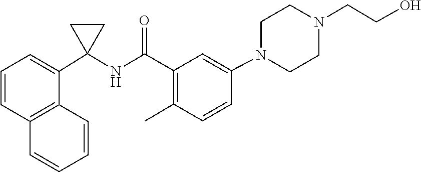 [SMILES: Cc1ccc(N2CCN(CCO)CC2)cc1C(=O)NC1(c2cccc3ccccc23)CC1] | A |
| 426 | 303 | 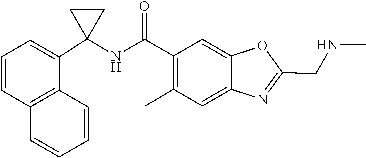 [SMILES: CNCc1nc2cc(C)c(C(=O)NC3(c4cccc5ccccc45)CC3)cc2o1] | B |
| 432 | 304 | 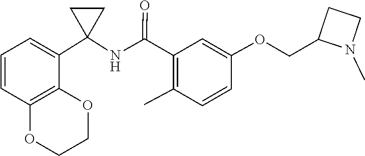 [SMILES: Cc1ccc(OCC2CCN2C)cc1C(=O)NC1(c2cccc3c2OCCO3)CC1] | B |
| 457 | 305 | 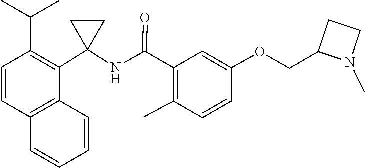 [SMILES: Cc1ccc(OCC2CCN2C)cc1C(=O)NC1(c2c(C(C)C)ccc3ccccc23)CC1] | B |
| 445 | 306 | 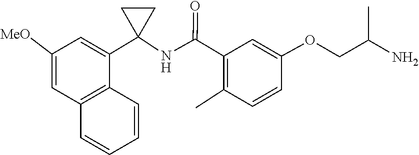 [SMILES: COc1cc(C2(NC(=O)c3cc(OCC(C)N)ccc3C)CC2)c2ccccc2c1] | A |
| 490 | 307 | 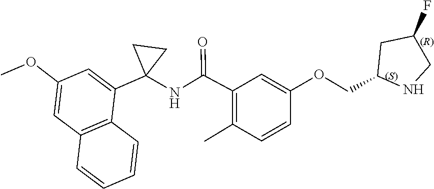 | A |
| 489 | 308 | 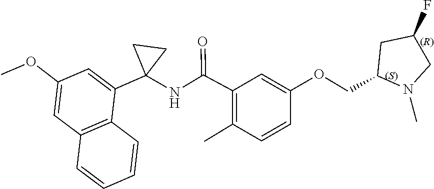 | A |
| 433 | 309 | 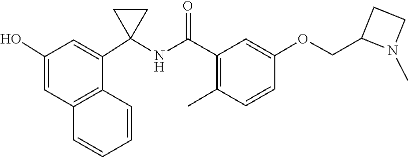 [SMILES: Cc1ccc(OCC2CCN2C)cc1C(=O)NC1(c2cc(O)cc3ccccc23)CC1] | A |
| 474 | 310 | 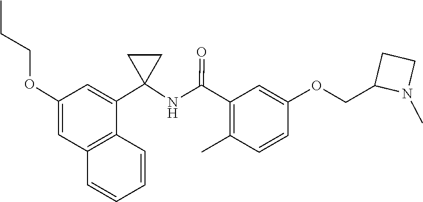 [SMILES: CCCOc1cc(C2(NC(=O)c3cc(OCC4CCN4C)ccc3C)CC2)c2ccccc2c1] | A |
| 461 | 311 | 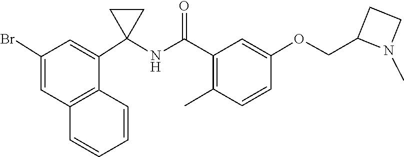 [SMILES: Cc1ccc(OCC2CCN2C)cc1C(=O)NC1(c2cc(Br)cc3ccccc23)CC1] | A |
| 541 | 312 | 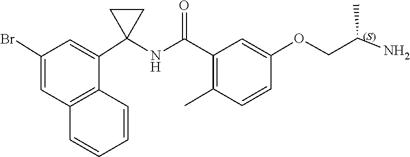 | A |
| 443 | 313 | 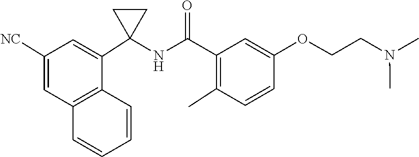 [SMILES: Cc1ccc(OCCN(C)C)cc1C(=O)NC1(c2cc(C#N)cc3ccccc23)CC1] | A |
| 484 | 314 | 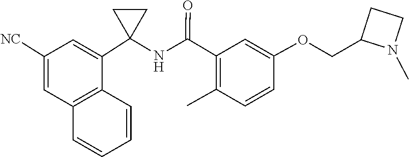 [SMILES: Cc1ccc(OCC2CCN2C)cc1C(=O)NC1(c2cc(C#N)cc3ccccc23)CC1] | A |
| 482 | 315 | 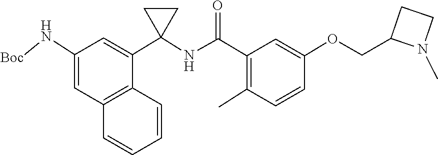 [SMILES: Cc1ccc(OCC2CCN2C)cc1C(=O)NC1(c2cc(NC(=O)OC(C)(C)C)cc3ccccc23)CC1] | A |
| 496 | 316 | 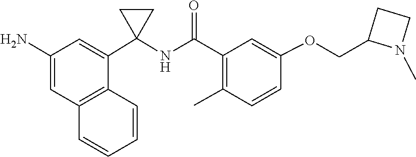 [SMILES: Cc1ccc(OCC2CCN2C)cc1C(=O)NC1(c2cc(N)cc3ccccc23)CC1] | A |
| 495 | 317 | 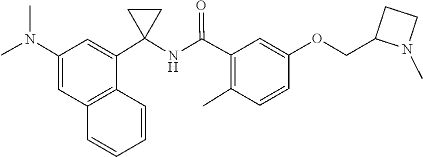 | A |
| 520 | 318 | 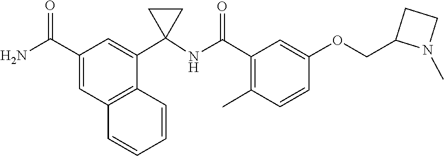 [SMILES: Cc1ccc(OCC2CCN2C)cc1C(=O)NC1(c2cc(C(N)=O)cc3ccccc23)CC1] | A |
| 481 | 319 | 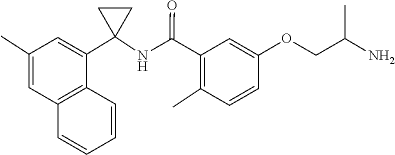 [SMILES: Cc1cc(C2(NC(=O)c3cc(OCC(C)N)ccc3C)CC2)c2ccccc2c1] | A |
| 501 | 320 | 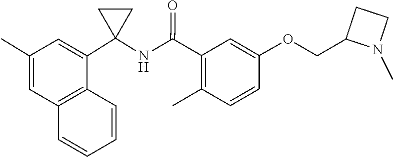 | A |
| 508 | 321 | 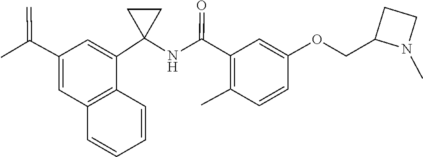 [SMILES: C=C(C)c1cc(C2(NC(=O)c3cc(OCC4CCN4C)ccc3C)CC2)c2ccccc2c1] | A |
| 521 | 322 | 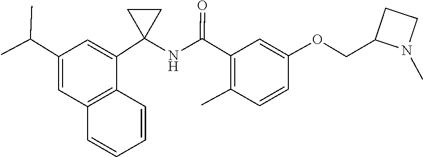 [SMILES: Cc1ccc(OCC2CCN2C)cc1C(=O)NC1(c2cc(C(C)C)cc3ccccc23)CC1] | A |
| 423 | 323 | 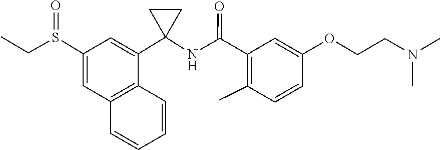 [SMILES: CCS(=O)c1cc(C2(NC(=O)c3cc(OCCN(C)C)ccc3C)CC2)c2ccccc2c1] | A |
| 471 | 324 | 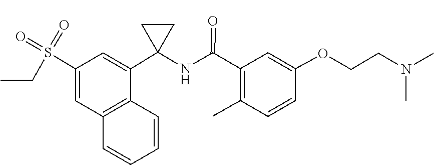 | B |
| 486 | 325 | 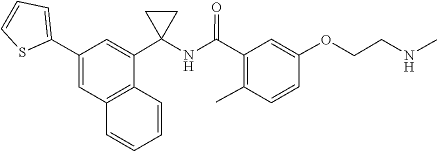 [SMILES: CNCCOc1ccc(C)c(C(=O)NC2(c3cc(-c4cccs4)cc4ccccc34)CC2)c1] | A |
| 539 | 326 | 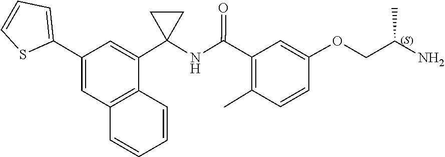 | A |
| 468 | 327 | 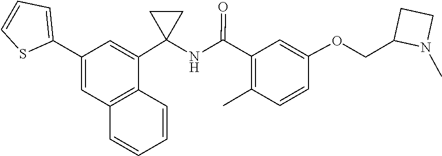 [SMILES: Cc1ccc(OCC2CCN2C)cc1C(=O)NC1(c2cc(-c3cccs3)cc3ccccc23)CC1] | A |
| 470 | 328 | 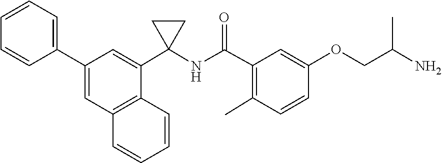 [SMILES: Cc1ccc(OCC(C)N)cc1C(=O)NC1(c2cc(-c3ccccc3)cc3ccccc23)CC1] | A |
| 475 | 329 | 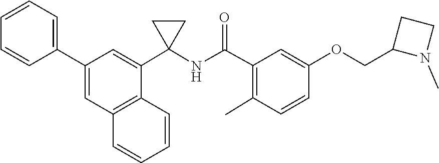 [SMILES: Cc1ccc(OCC2CCN2C)cc1C(=O)NC1(c2cc(-c3ccccc3)cc3ccccc23)CC1] | A |
| 477 | 330 | 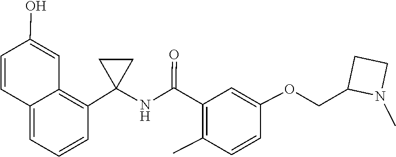 [SMILES: Cc1ccc(OCC2CCN2C)cc1C(=O)NC1(c2cccc3ccc(O)cc23)CC1] | A |
| 465 | 331 | 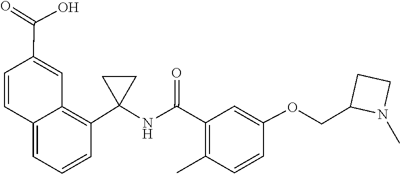 [SMILES: Cc1ccc(OCC2CCN2C)cc1C(=O)NC1(c2cccc3ccc(C(=O)O)cc23)CC1] | C |
| 483 | 332 | 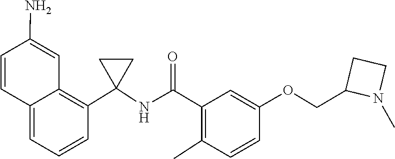 [SMILES: Cc1ccc(OCC2CCN2C)cc1C(=O)NC1(c2cccc3ccc(N)cc23)CC1] | A |
| 434 | 333 | 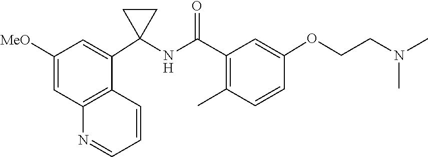 [SMILES: COc1cc(C2(NC(=O)c3cc(OCCN(C)C)ccc3C)CC2)c2cccnc2c1] | A |
| 530 | 334 | 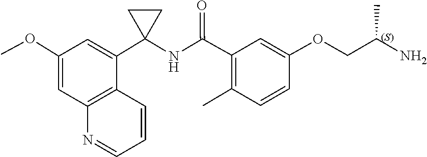 | A |
| 526 | 335 | 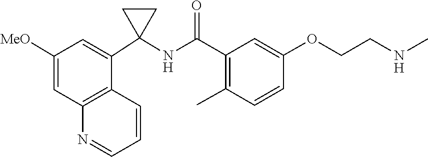 [SMILES: CNCCOc1ccc(C)c(C(=O)NC2(c3cc(OC)cc4ncccc34)CC2)c1] | A |
| 560 | 336 | 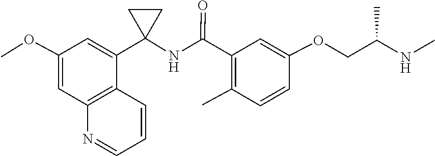 | A |
| 575 | 337 | 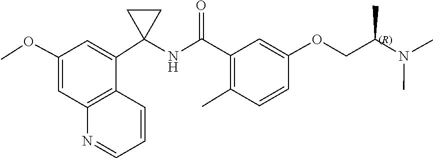 | A |
| 511 | 338 | 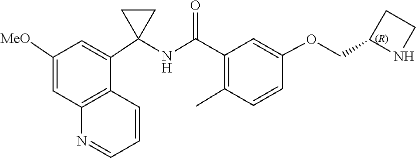 | A |
| 527 | 339 | 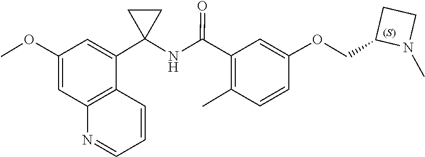 | A |
| 462 | 340 | 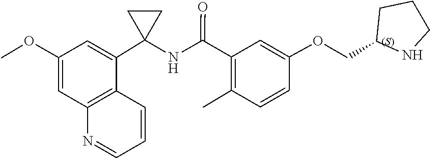 | A |
| 528 | 341 | 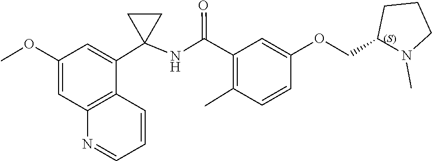 | A |
| 550 | 342 | 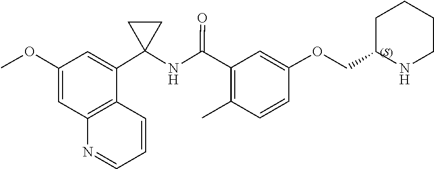 | A |
| 561 | 343 | 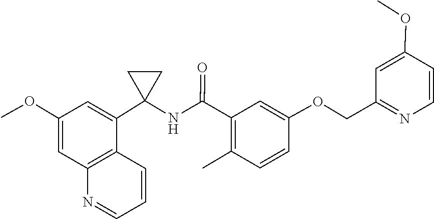 [SMILES: COc1ccnc(COc2ccc(C)c(C(=O)NC3(c4cc(OC)cc5ncccc45)CC3)c2)c1] | B |
| 584 | 344 | 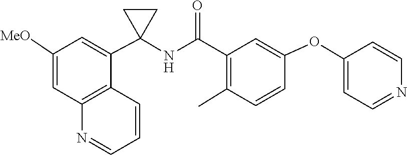 [SMILES: COc1cc(C2(NC(=O)c3cc(Oc4ccncc4)ccc3C)CC2)c2cccnc2c1] | B |
| 597 | 345 | 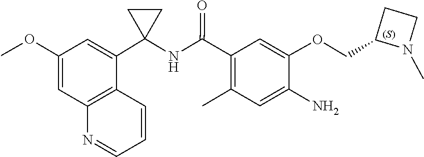 | A |
| 587 | 346 | 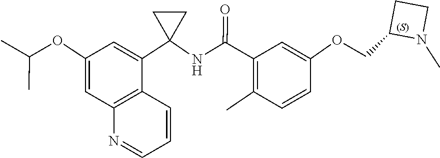 | A |
| 598 | 347 | 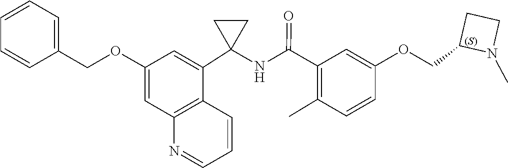 | A |
| 563 | 348 | 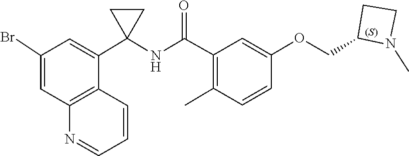 | A |
| 497 | 349 | 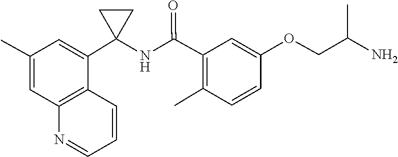 [SMILES: Cc1cc(C2(NC(=O)c3cc(OCC(C)N)ccc3C)CC2)c2cccnc2c1] | A |
| 581 | 350 | 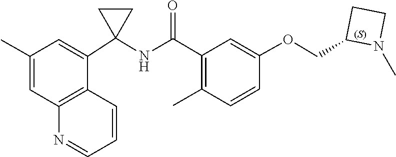 | A |
| 600 | 351 | 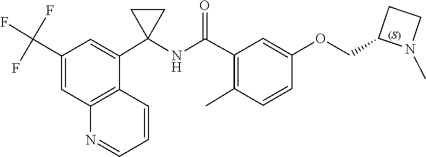 | A |
| 568 | 352 | 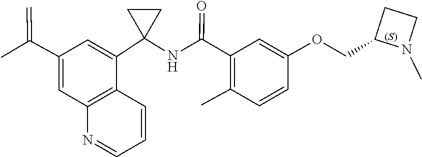 | A |
| 551 | 353 | 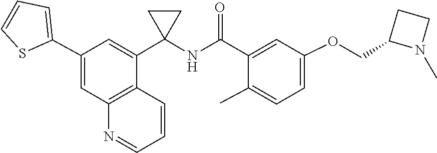 [SMILES: Cc1ccc(OC[C@@H]2CCN2C)cc1C(=O)NC1(c2cc(-c3cccs3)cc3ncccc23)CC1] | A |
| 588 | 354 | 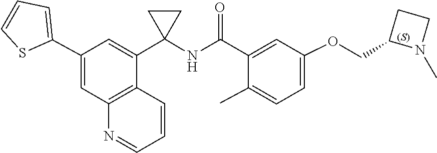 | A |
| 436 | 355 | 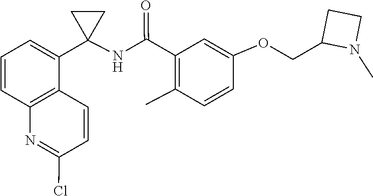 [SMILES: Cc1ccc(OCC2CCN2C)cc1C(=O)NC1(c2cccc3nc(Cl)ccc23)CC1] | A |
| 529 | 356 | 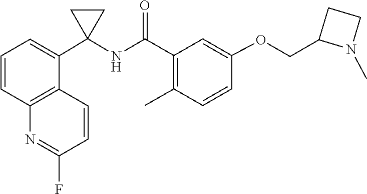 [SMILES: Cc1ccc(OCC2CCN2C)cc1C(=O)NC1(c2cccc3nc(F)ccc23)CC1] | A |
| 547 | 357 | 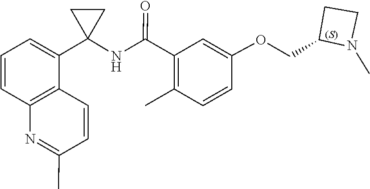 | A |
| 545 | 358 | 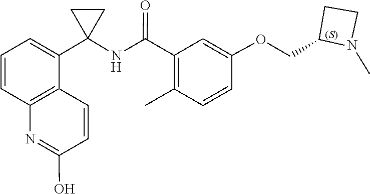 | A |
| 544 | 359 | 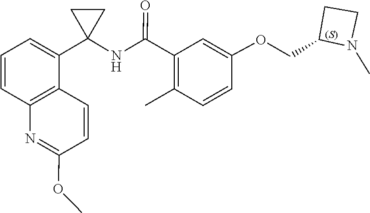 | A |
| 548 | 360 | 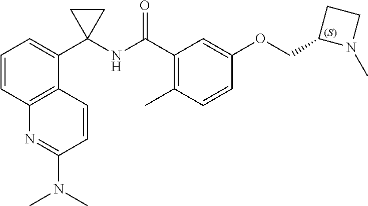 | A |
| 555 | 361 | 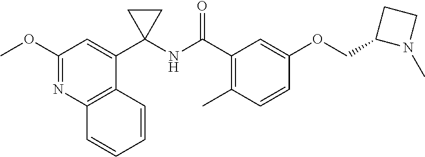 | A |
| 467 | 362 | 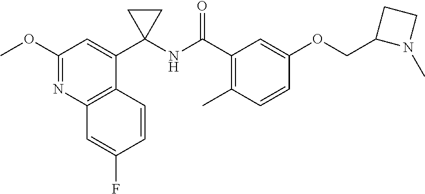 | A |
| 565 | 363 | 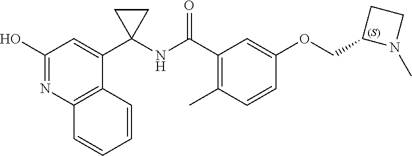 | A |
| 542 | 364 | 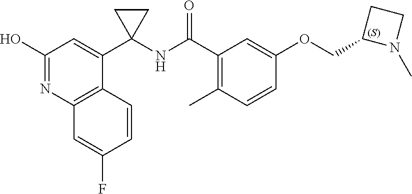 | A |
| 586 | 365 | 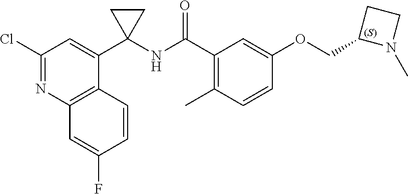 | A |
| 538 | 366 | 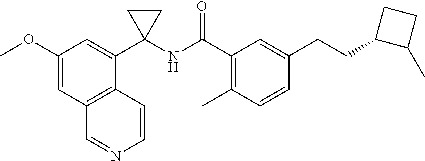 | A |
| 509 | 367 | 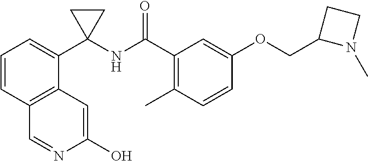 [SMILES: Cc1ccc(OCC2CCN2C)cc1C(=O)NC1(c2cccc3cnc(O)cc23)CC1] | B |
| 549 | 368 | 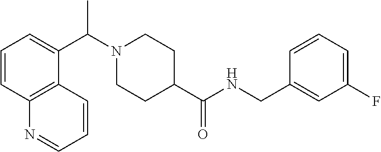 [SMILES: CC(c1cccc2ncccc12)N1CCC(C(=O)NCc2cccc(F)c2)CC1] | D |
| 522 | 369 | 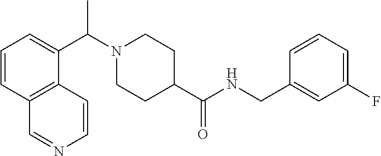 [SMILES: CC(c1cccc2cnccc12)N1CCC(C(=O)NCc2cccc(F)c2)CC1] | D |
| 524 | 370 | 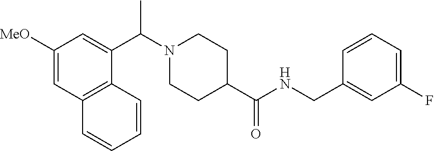 [SMILES: COc1cc(C(C)N2CCC(C(=O)NCc3cccc(F)c3)CC2)c2ccccc2c1] | B |
| 106 | 371 | 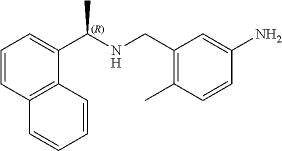 | D |
| 178 | 372 | 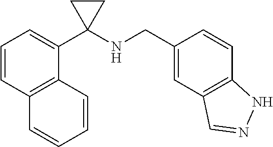 [SMILES: c1ccc2c(C3(NCc4ccc5[nH]ncc5c4)CC3)cccc2c1] | D |
| 184 | 373 | 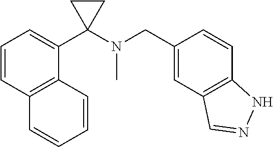 [SMILES: CN(Cc1ccc2[nH]ncc2c1)C1(c2cccc3ccccc23)CC1] | D |
| 208 | 374 | 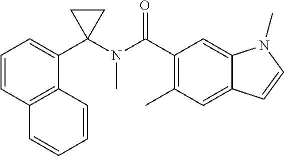 [SMILES: Cc1cc2ccn(C)c2cc1C(=O)N(C)C1(c2cccc3ccccc23)CC1] | D |
| 179 | 375 | 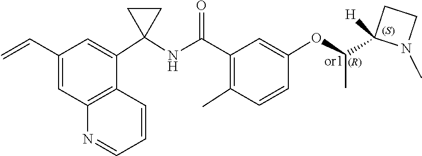 | D |
| 577 | 376 | 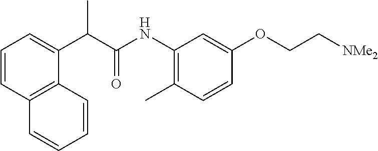 [SMILES: Cc1ccc(OCCN(C)C)cc1NC(=O)C(C)c1cccc2ccccc12] | D |
| 580 | 377 | 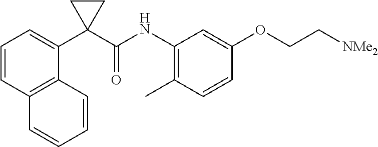 [SMILES: Cc1ccc(OCCN(C)C)cc1NC(=O)C1(c2cccc3ccccc23)CC1] | D |
| 111 | 378 | 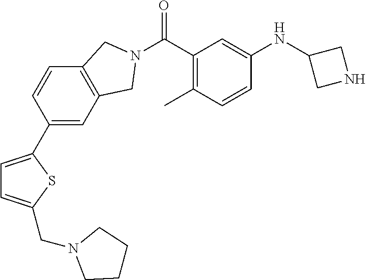 [SMILES: Cc1ccc(NC2CNC2)cc1C(=O)N1Cc2ccc(-c3ccc(CN4CCCC4)s3)cc2C1] | D |
| 116 | 379 | 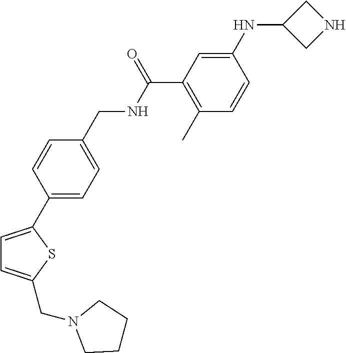 [SMILES: Cc1ccc(NC2CNC2)cc1C(=O)NCc1ccc(-c2ccc(CN3CCCC3)s2)cc1] | D |
| 161 | 380 | 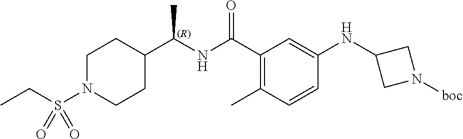 | D |
| 166 | 381 | 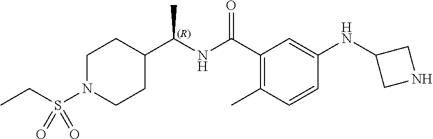 | D |
| 108 | 382 | 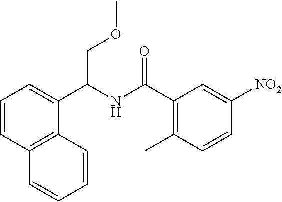 [SMILES: COCC(NC(=O)c1cc([N+](=O)[O-])ccc1C)c1cccc2ccccc12] | D |
| 118 | 383 | 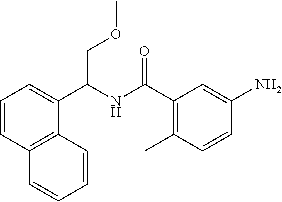 [SMILES: COCC(NC(=O)c1cc(N)ccc1C)c1cccc2ccccc12] | D |
| 105 | 384 | 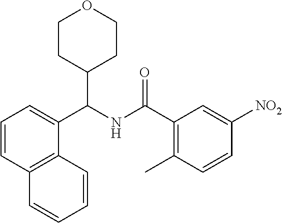 [SMILES: Cc1ccc([N+](=O)[O-])cc1C(=O)NC(c1cccc2ccccc12)C1CCOCC1] | D |
| 109 | 385 | 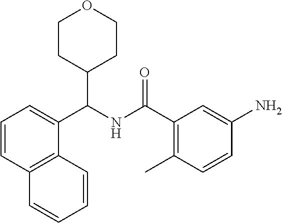 [SMILES: Cc1ccc(N)cc1C(=O)NC(c1cccc2ccccc12)C1CCOCC1] | D |
| 242 | 386 | 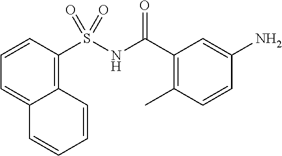 [SMILES: Cc1ccc(N)cc1C(=O)NS(=O)(=O)c1cccc2ccccc12] | D |
| 138 | 387 | 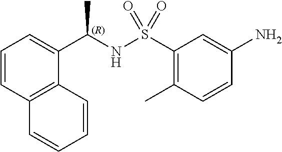 | D |
| 217 | 388 | 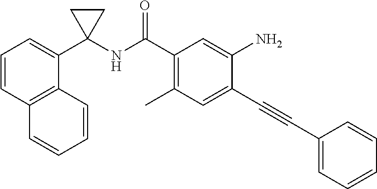 [SMILES: Cc1cc(C#Cc2ccccc2)c(N)cc1C(=O)NC1(c2cccc3ccccc23)CC1] | D |
| 624 | 389 | 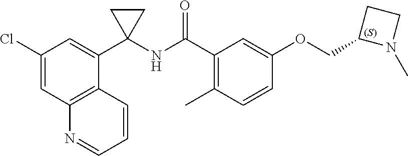 | A |
| 729 | 390 | 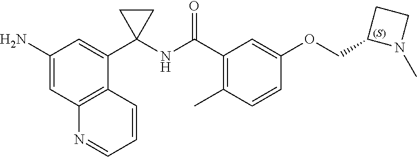 | A |
| 627 | 391 | 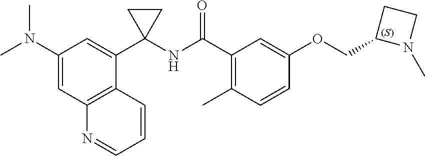 | A |
| 611 | 392 | 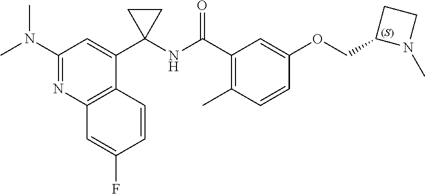 | A |
| 743 | 393 | 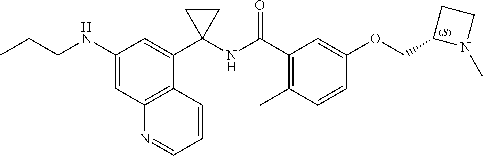 | A |
| 744 | 394 | 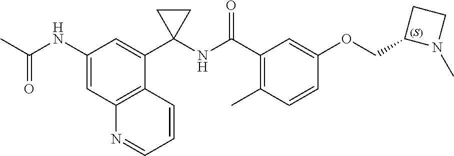 | A |
| 786 | 395 | 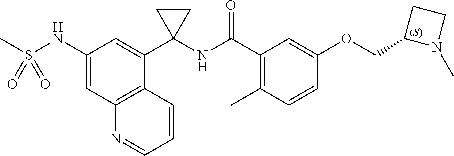 | A |
| 757 | 396 | 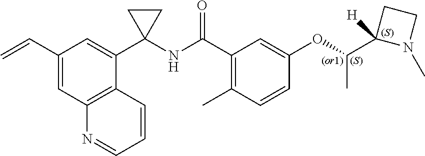 | A |
| 713 | 397 | 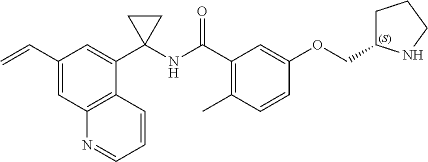 | A |
| 748 | 398 | 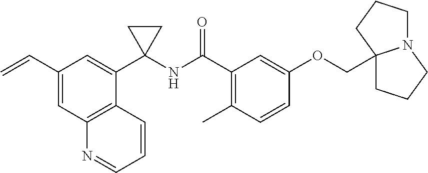 [SMILES: C=Cc1cc(C2(NC(=O)c3cc(OCC45CCCN4CCC5)ccc3C)CC2)c2cccnc2c1] | A |
| 749 | 399 | 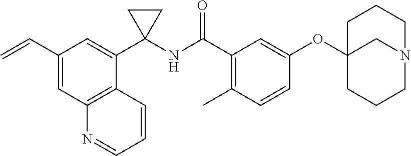 [SMILES: C=Cc1cc(C2(NC(=O)c3cc(OC45CCCN(CCC4)C5)ccc3C)CC2)c2cccnc2c1] | A |
| 800 | 400 | 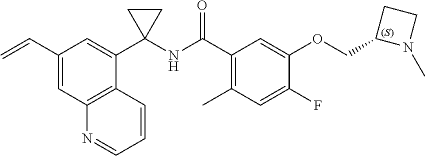 | A |
| 726 | 401 | 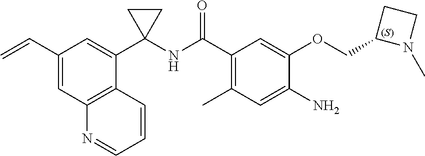 | A |
| 746 | 402 | 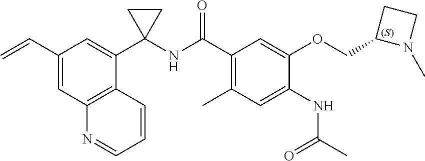 | A |
| 785 | 403 | 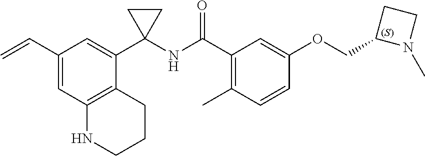 | A |
| 796 | 404 | 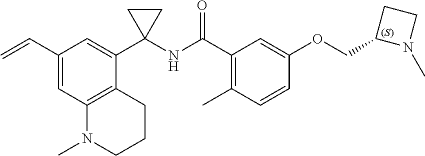 | A |
| 660 | 405 | 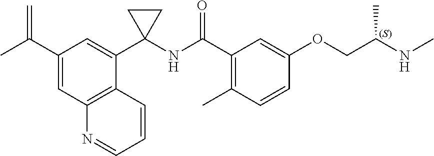 | A |
| 638 | 406 | 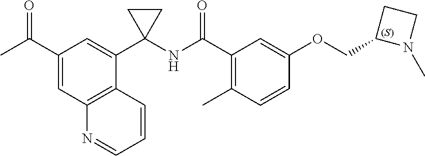 | A |
| 676 | 407 | 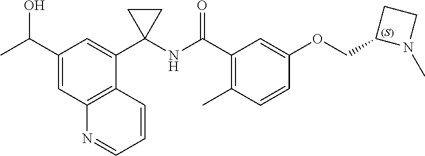 | A |
| 697 | 408 | 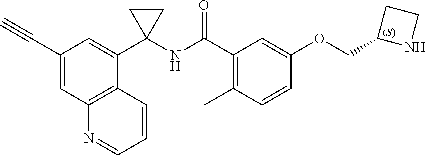 | A |
| 620 | 409 | 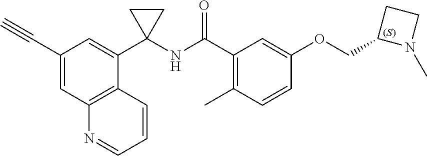 | A |
| 696 | 410 | 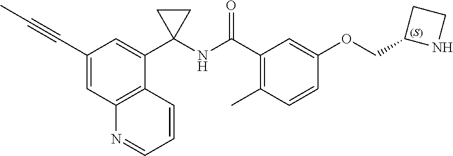 | A |
| 803 | 411 | 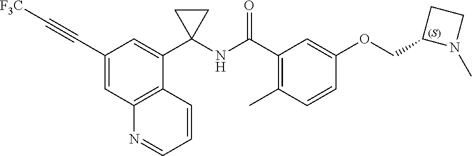 | A |
| 738 | 412 | 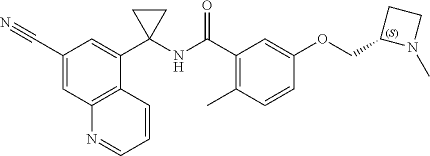 | A |
| 677 | 413 | 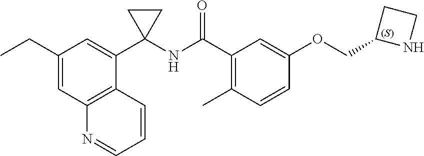 | A |
| 692 | 414 | 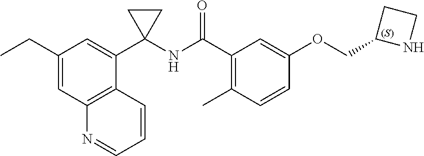 | A |
| 761 | 415 | 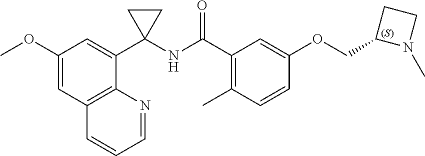 | B |
| 626 | 416 | 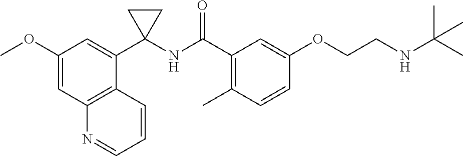 [SMILES: COc1cc(C2(NC(=O)c3cc(OCCNC(C)(C)C)ccc3C)CC2)c2cccnc2c1] | A |
| 797 | 417 | 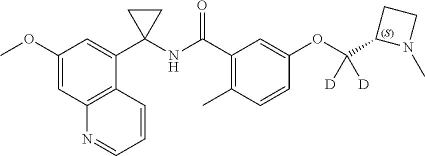 | A |
| 732 | 418 | 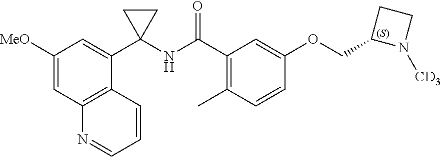 | A |
| 775 | 419 | 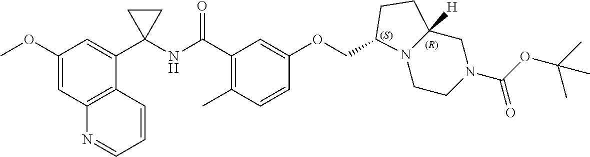 | A |
| 776 | 419 | 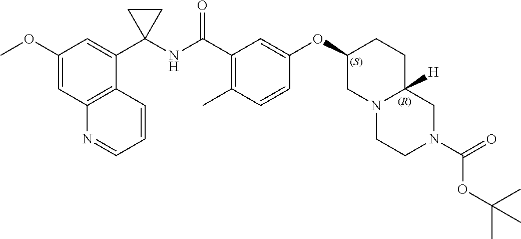 | A |
| 789 | 420 | 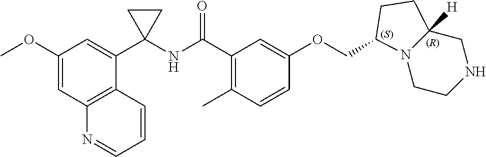 | A |
| 790 | 420 | 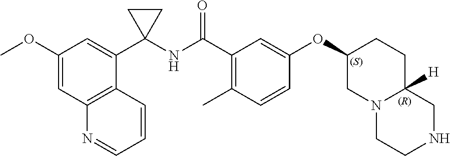 | A |
| 783 | 421 | 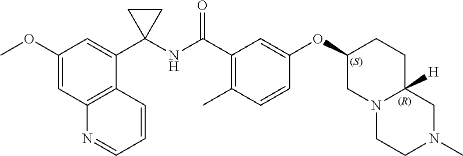 | A |
| 784 | 421 | 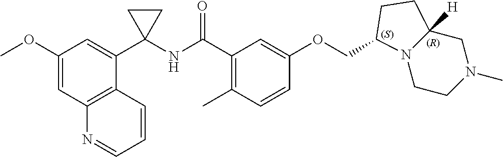 | A |
| 799 | 422 | 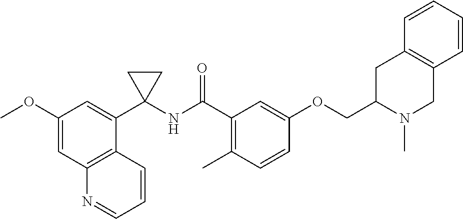 [SMILES: COc1cc(C2(NC(=O)c3cc(OCC4Cc5ccccc5CN4C)ccc3C)CC2)c2cccnc2c1] | A |
| 794 | 423 | 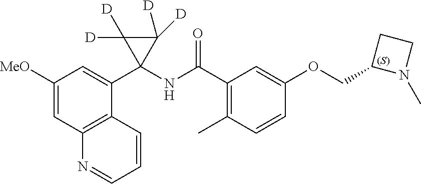 | A |
| 750 | 424 | 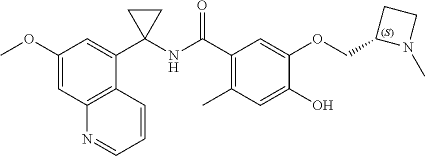 | A |
| 737 | 425 | 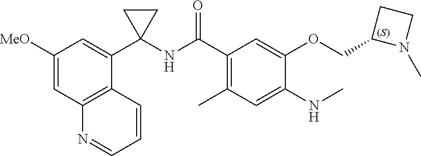 | A |
| 625 | 426 | 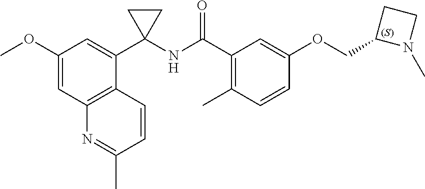 | A |
| 791 | 427 | 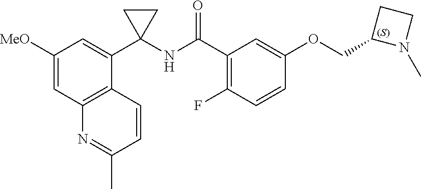 | B |
| 817 | 428 | 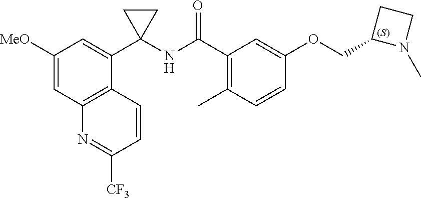 | A |
| 809 | 429 | 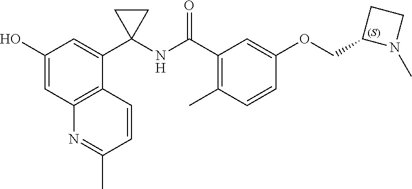 | A |
| 795 | 430 | 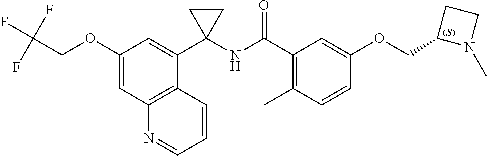 | A |
| 815 | 431 | 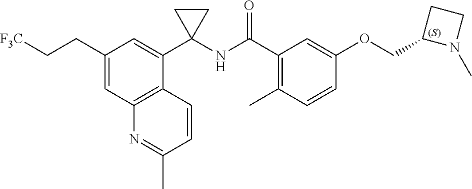 | A |
| 648 | 432 | 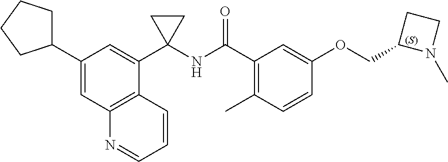 | A |
| 683 | 433 | 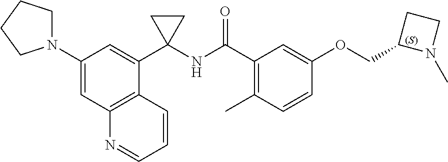 | A |
| 630 | 434 | 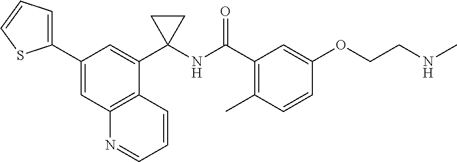 [SMILES: CNCCOc1ccc(C)c(C(=O)NC2(c3cc(-c4cccs4)cc4ncccc34)CC2)c1] | A |
| 608 | 435 | 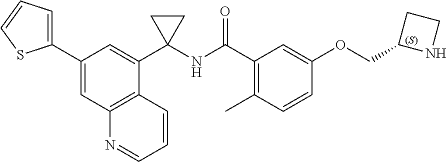 | A |
| 753 | 436 | 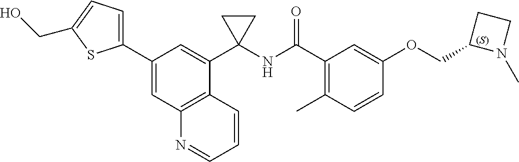 | A |
| 780 | 437 | 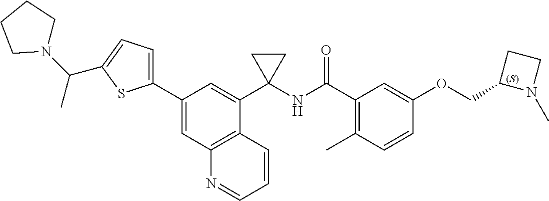 | A |
| 669 | 438 | 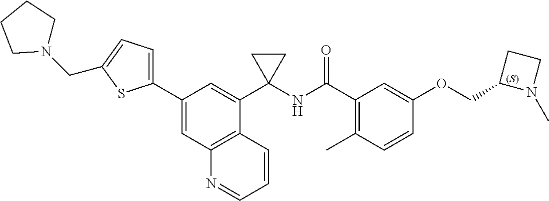 | A |
| 767 | 439 | 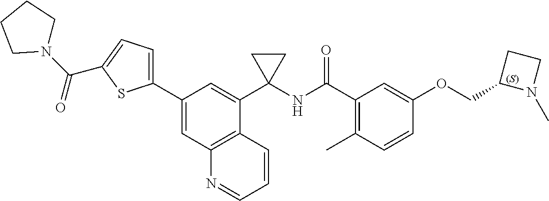 | D |
| 812 | 440 | 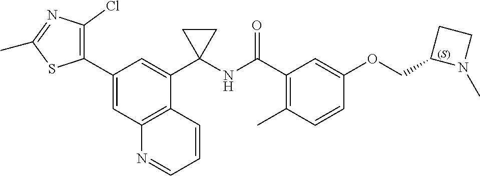 | A |
| 814 | 441 | 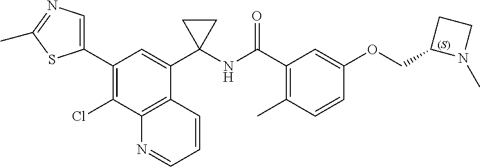 | A |
| 782 | 442 | 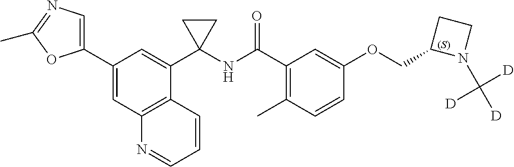 | A |
| 806 | 443 | 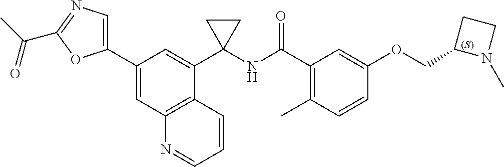 | A |
| 619 | 444 | 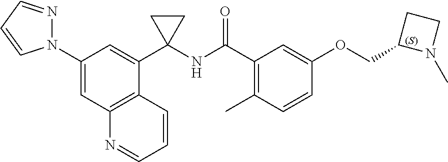 | A |
| 653 | 445 | 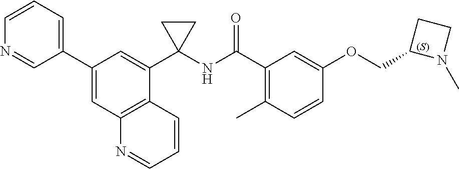 | |
| 689 | 446 | 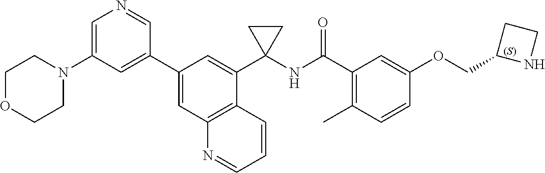 | A |
| 821 | 447 | 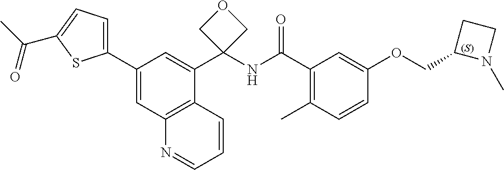 | A |
| 808 | 448 | 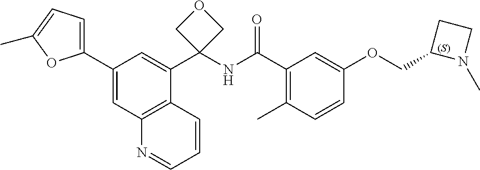 | A |
| 181 | 449 | 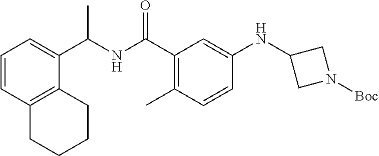 [SMILES: Cc1ccc(NC2CN(C(=O)OC(C)(C)C)C2)cc1C(=O)NC(C)c1cccc2c1CCCC2] | D |
| 104 | 450 | 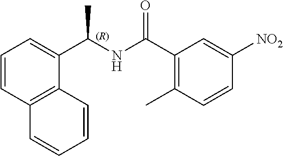 | D |
| 188 | 451 | 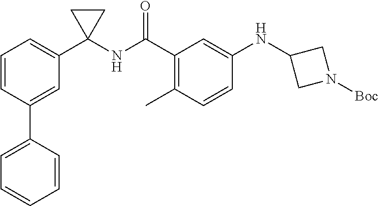 [SMILES: Cc1ccc(NC2CN(C(=O)OC(C)(C)C)C2)cc1C(=O)NC1(c2cccc(-c3ccccc3)c2)CC1] | D |
| 127 | 452 | 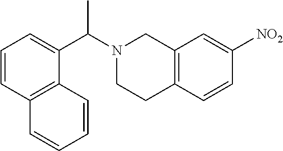 [SMILES: CC(c1cccc2ccccc12)N1CCc2ccc([N+](=O)[O-])cc2C1] | D |
| 739 | 453 | 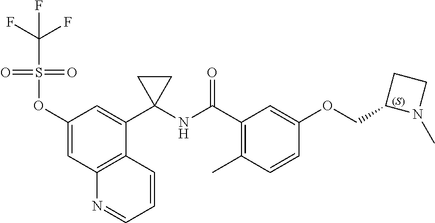 | A |
| 110 | 454 | 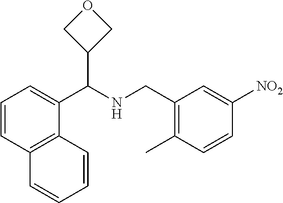 [SMILES: Cc1ccc([N+](=O)[O-])cc1CNC(c1cccc2ccccc12)C1COC1] | D |
| 123 | 455 | 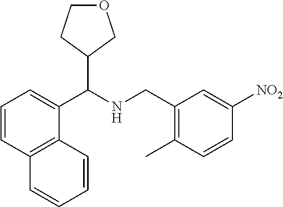 [SMILES: Cc1ccc([N+](=O)[O-])cc1CNC(c1cccc2ccccc12)C1CCOC1] | D |
| 113 | 456 | 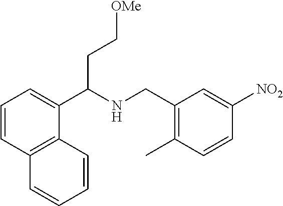 [SMILES: COCCC(NCc1cc([N+](=O)[O-])ccc1C)c1cccc2ccccc12] | D |
| 114 | 457 | 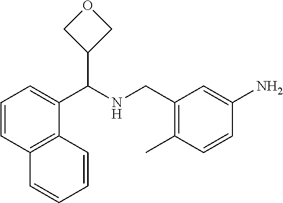 [SMILES: Cc1ccc(N)cc1CNC(c1cccc2ccccc12)C1COC1] | D |
| 117 | 458 | 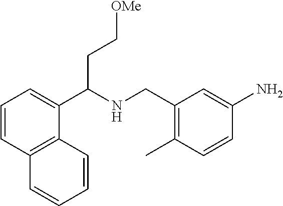 [SMILES: COCCC(NCc1cc(N)ccc1C)c1cccc2ccccc12] | D |
| 182 | 459 | 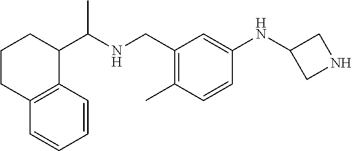 [SMILES: Cc1ccc(NC2CNC2)cc1CNC(C)C1CCCc2ccccc21] | D |
| 607 | 460 | 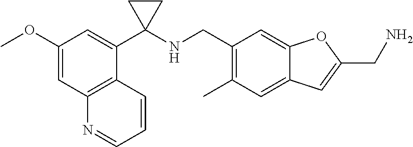 [SMILES: COc1cc(C2(NCc3cc4oc(CN)cc4cc3C)CC2)c2cccnc2c1] | A |
| 606 | 461 | 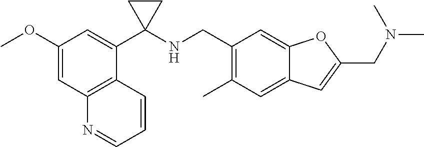 | A |
| 513 | 462 | 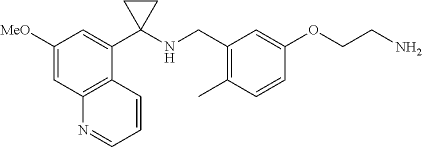 [SMILES: COc1cc(C2(NCc3cc(OCCN)ccc3C)CC2)c2cccnc2c1] | A |
| 430 | 463 | 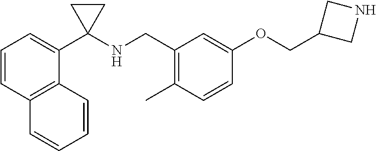 [SMILES: Cc1ccc(OCC2CNC2)cc1CNC1(c2cccc3ccccc23)CC1] | A |
| 428 | 464 | 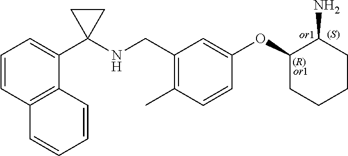 | B |
| 429 | 465 | 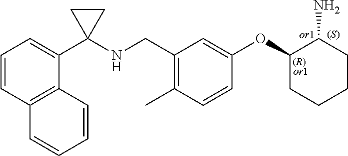 | B |
| 277 | 466 | 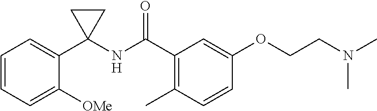 [SMILES: COc1ccccc1C1(NC(=O)c2cc(OCCN(C)C)ccc2C)CC1] | D |
| 278 | 467 | 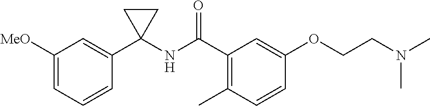 [SMILES: COc1cccc(C2(NC(=O)c3cc(OCCN(C)C)ccc3C)CC2)c1] | D |
| 569 | 468 | 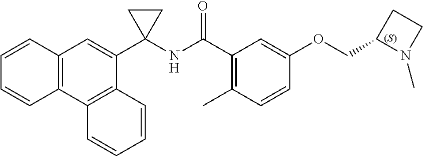 | D |
| 427 | 469 | 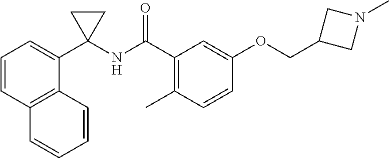 [SMILES: Cc1ccc(OCC2CN(C)C2)cc1C(=O)NC1(c2cccc3ccccc23)CC1] | A |
| 306 | 470 | 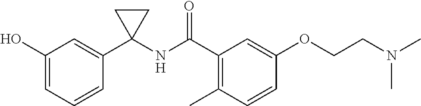 [SMILES: Cc1ccc(OCCN(C)C)cc1C(=O)NC1(c2cccc(O)c2)CC1] | D |
| 572 | 471 | 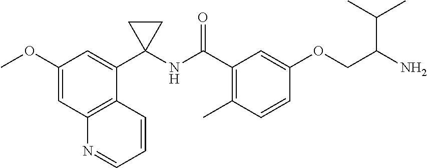 [SMILES: COc1cc(C2(NC(=O)c3cc(OCC(N)C(C)C)ccc3C)CC2)c2cccnc2c1] | A |
| 567 | 472 | 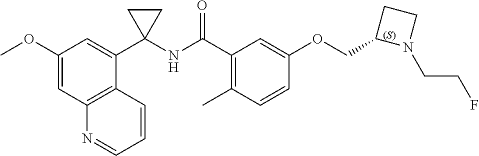 | A |
| 571 | 473 | 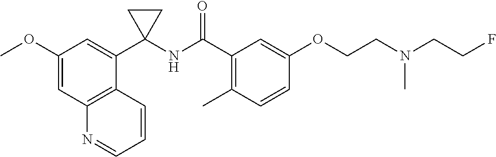 [SMILES: COc1cc(C2(NC(=O)c3cc(OCCN(C)CCF)ccc3C)CC2)c2cccnc2c1] | A |
| 444 | 474 | 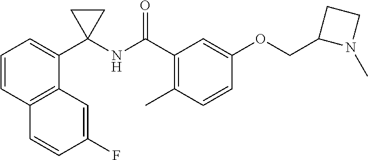 [SMILES: Cc1ccc(OCC2CCN2C)cc1C(=O)NC1(c2cccc3ccc(F)cc23)CC1] | A |
| 559 | 475 | 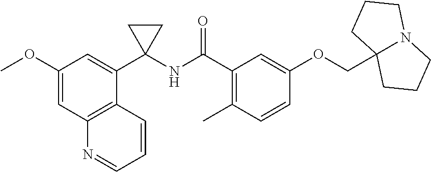 [SMILES: COc1cc(C2(NC(=O)c3cc(OCC45CCCN4CCC5)ccc3C)CC2)c2cccnc2c1] | A |
| 596 | 476 | 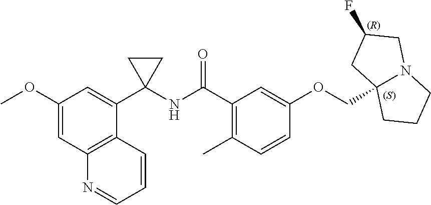 | A |
| 454 | 477 | 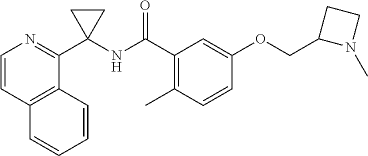 [SMILES: Cc1ccc(OCC2CCN2C)cc1C(=O)NC1(c2nccc3ccccc23)CC1] | B |
| 473 | 478 | 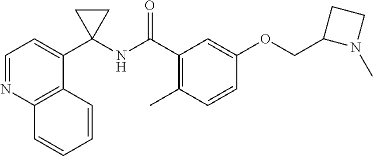 [SMILES: Cc1ccc(OCC2CCN2C)cc1C(=O)NC1(c2ccnc3ccccc23)CC1] | A |
| 456 | 479 | 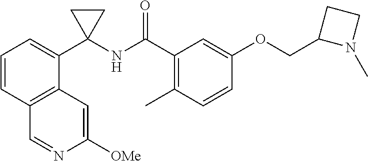 [SMILES: COc1cc2c(C3(NC(=O)c4cc(OCC5CCN5C)ccc4C)CC3)cccc2cn1] | A |
| 458 | 480 | 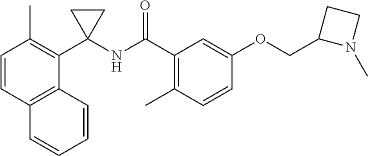 [SMILES: Cc1ccc(OCC2CCN2C)cc1C(=O)NC1(c2c(C)ccc3ccccc23)CC1] | A |
| 507 | 481 | 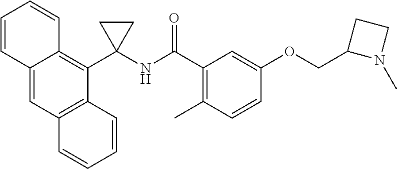 [SMILES: Cc1ccc(OCC2CCN2C)cc1C(=O)NC1(c2c3ccccc3cc3ccccc23)CC1] | B |
| 488 | 482 | 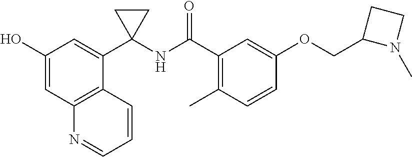 [SMILES: Cc1ccc(OCC2CCN2C)cc1C(=O)NC1(c2cc(O)cc3ncccc23)CC1] | A |
| 617 | 483 | 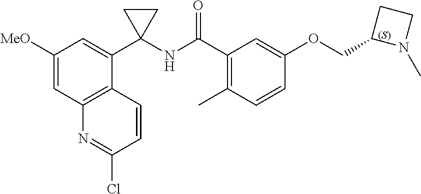 | A |
| 498 | 484 | 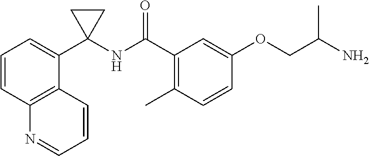 [SMILES: Cc1ccc(OCC(C)N)cc1C(=O)NC1(c2cccc3ncccc23)CC1] | A |
| 446 | 485 | 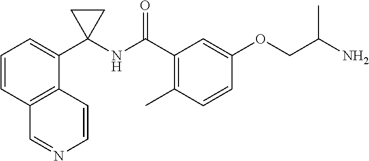 [SMILES: Cc1ccc(OCC(C)N)cc1C(=O)NC1(c2cccc3cnccc23)CC1] | A |
| 566 | 486 | 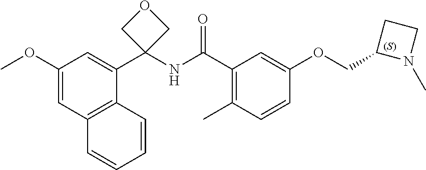 | A |
| 464 | 487 | 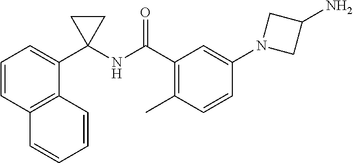 [SMILES: Cc1ccc(N2CC(N)C2)cc1C(=O)NC1(c2cccc3ccccc23)CC1] | A |
| 485 | 488 | 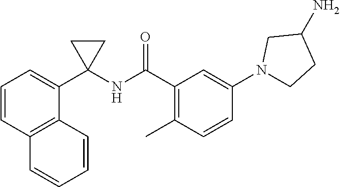 [SMILES: Cc1ccc(N2CCC(N)C2)cc1C(=O)NC1(c2cccc3ccccc23)CC1] | A |
| 506 | 489 | 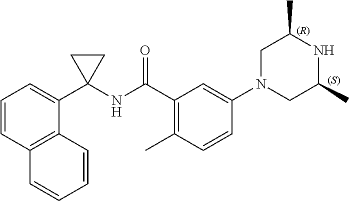 | A |
| 479 | 490 | 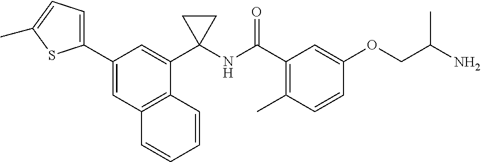 [SMILES: Cc1ccc(-c2cc(C3(NC(=O)c4cc(OCC(C)N)ccc4C)CC3)c3ccccc3c2)s1] | A |
| 502 | 491 | 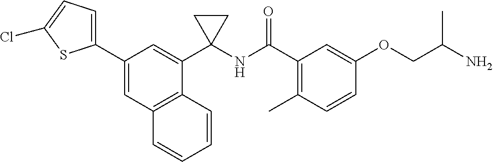 [SMILES: Cc1ccc(OCC(C)N)cc1C(=O)NC1(c2cc(-c3ccc(Cl)s3)cc3ccccc23)CC1] | A |
| 480 | 492 | 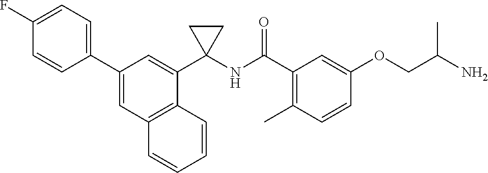 [SMILES: Cc1ccc(OCC(C)N)cc1C(=O)NC1(c2cc(-c3ccc(F)cc3)cc3ccccc23)CC1] | A |
| 478 | 493 | 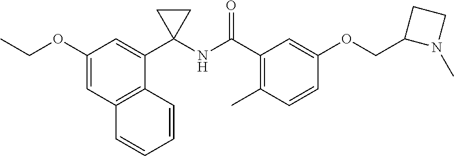 [SMILES: CCOc1cc(C2(NC(=O)c3cc(OCC4CCN4C)ccc3C)CC2)c2ccccc2c1] | A |
| 476 | 494 | 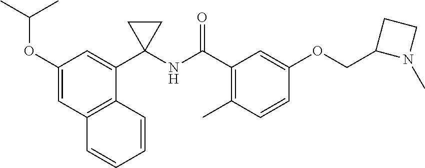 [SMILES: Cc1ccc(OCC2CCN2C)cc1C(=O)NC1(c2cc(OC(C)C)cc3ccccc23)CC1] | A |
| 787 | 495 | 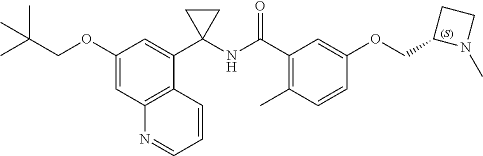 | A |
| 493 | 496 | 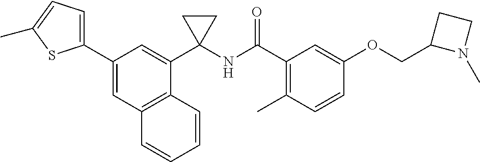 | A |
| 514 | 497 | 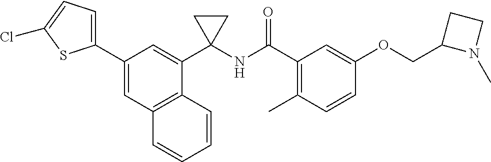 [SMILES: Cc1ccc(OCC2CCN2C)cc1C(=O)NC1(c2cc(-c3ccc(Cl)s3)cc3ccccc23)CC1] | A |
| 518 | 498 | 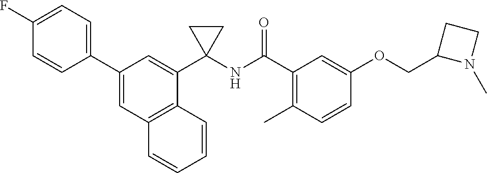 [SMILES: Cc1ccc(OCC2CCN2C)cc1C(=O)NC1(c2cc(-c3ccc(F)cc3)cc3ccccc23)CC1] | A |
| 500 | 499 | 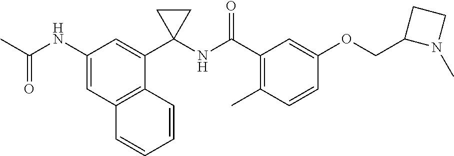 [SMILES: CC(=O)Nc1cc(C2(NC(=O)c3cc(OCC4CCN4C)ccc3C)CC2)c2ccccc2c1] | A |
| 510 | 500 | 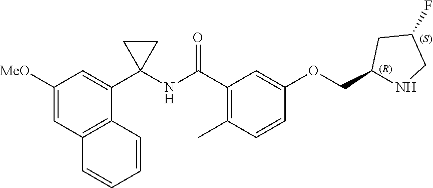 | A |
| 512 | 501 | 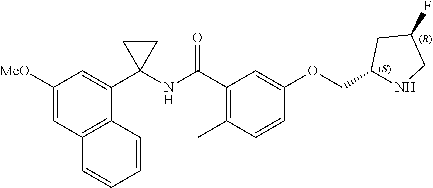 | A |
| 556 | 502 | 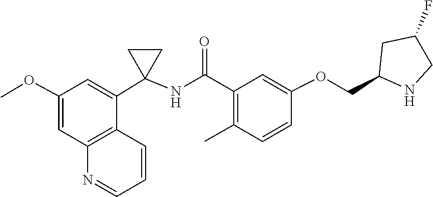 [SMILES: COc1cc(C2(NC(=O)c3cc(OC[C@H]4C[C@H](F)CN4)ccc3C)CC2)c2cccnc2c1] | A |
| 517 | 503 | 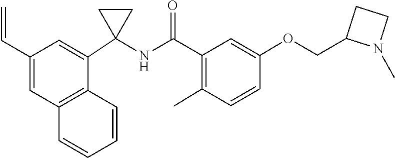 [SMILES: C=Cc1cc(C2(NC(=O)c3cc(OCC4CCN4C)ccc3C)CC2)c2ccccc2c1] | A |
| 503 | 504 | 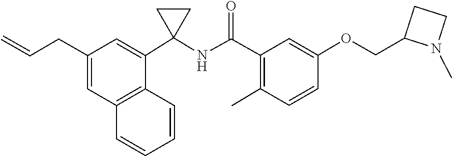 [SMILES: C=CCc1cc(C2(NC(=O)c3cc(OCC4CCN4C)ccc3C)CC2)c2ccccc2c1] | A |
| 578 | 505 | 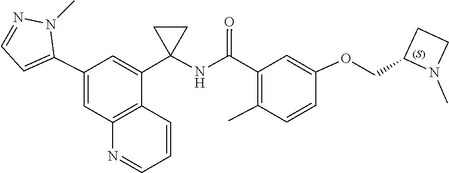 | A |
| 554 | 506 | 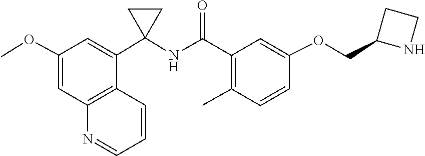 [SMILES: COc1cc(C2(NC(=O)c3cc(OC[C@H]4CCN4)ccc3C)CC2)c2cccnc2c1] | A |
| 516 | 507 | 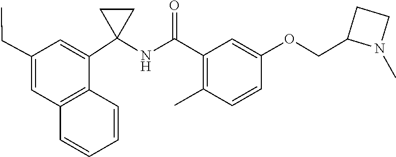 [SMILES: CCc1cc(C2(NC(=O)c3cc(OCC4CCN4C)ccc3C)CC2)c2ccccc2c1] | A |
| 504 | 508 | 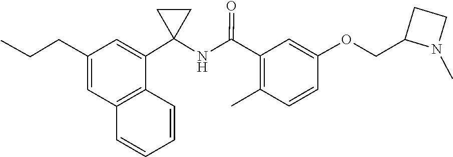 [SMILES: CCCc1cc(C2(NC(=O)c3cc(OCC4CCN4C)ccc3C)CC2)c2ccccc2c1] | A |
| 558 | 509 | 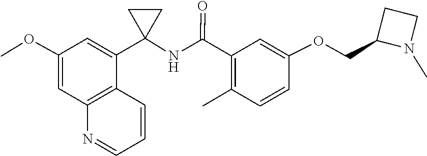 | A |
| 531 | 510 | 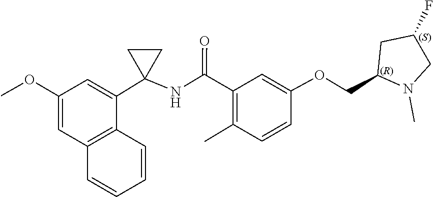 | A |
| 532 | 511 | 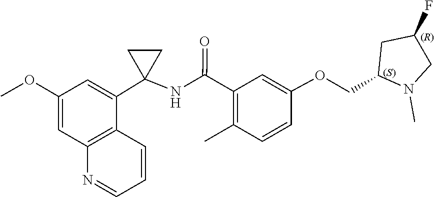 | A |
| 557 | 512 | 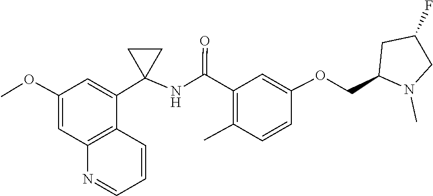 [SMILES: COc1cc(C2(NC(=O)c3cc(OC[C@H]4C[C@H](F)CN4C)ccc3C)CC2)c2cccnc2c1] | A |
| 553 | 513 | 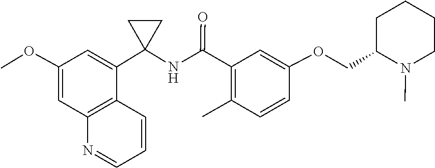 [SMILES: COc1cc(C2(NC(=O)c3cc(OC[C@@H]4CCCCN4C)ccc3C)CC2)c2cccnc2c1] | A |
| 535 | 514 | 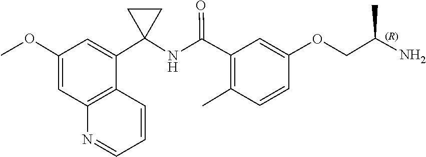 | A |
| 536 | 515 | 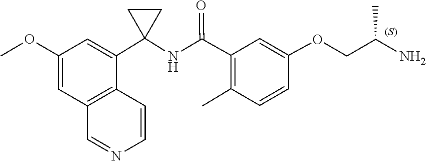 | A |
| 537 | 516 | 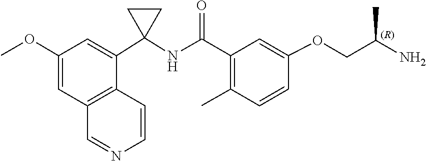 | A |
| 543 | 517 | 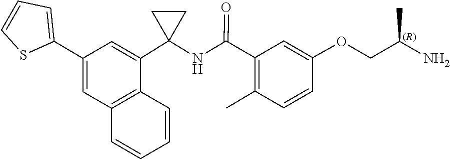 | A |
| 540 | 518 | 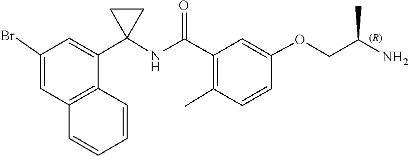 | A |
| 552 | 519 | 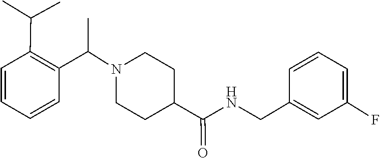 [SMILES: CC(C)c1ccccc1C(C)N1CCC(C(=O)NCc2cccc(F)c2)CC1] | D |
| 593 | 520 | 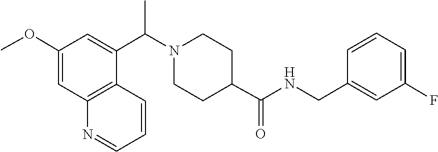 [SMILES: COc1cc(C(C)N2CCC(C(=O)NCc3cccc(F)c3)CC2)c2cccnc2c1] | C |
| 599 | 521 | 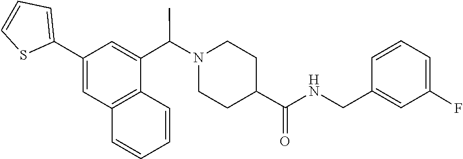 [SMILES: CC(c1cc(-c2cccs2)cc2ccccc12)N1CCC(C(=O)NCc2cccc(F)c2)CC1] | C |
| 573 | 522 | 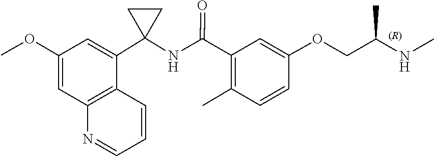 | A |
| 562 | 523 | 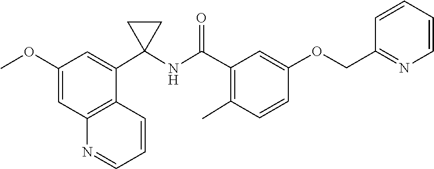 [SMILES: COc1cc(C2(NC(=O)c3cc(OCc4ccccn4)ccc3C)CC2)c2cccnc2c1] | B |
| 579 | 524 | 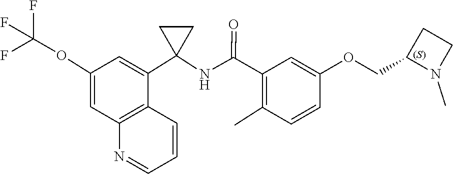 | A |
| 570 | 525 | 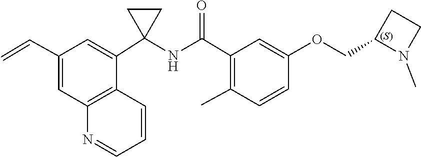 | A |
| 603 | 526 | 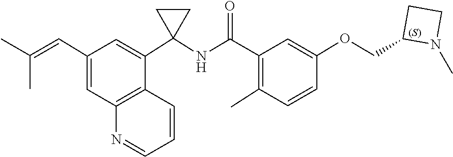 | A |
| 650 | 527 | 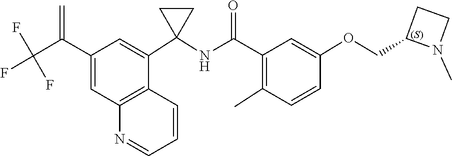 | A |
| 668 | 528 | 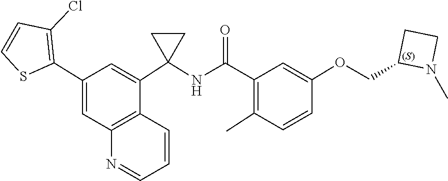 | A |
| 632 | 529 | 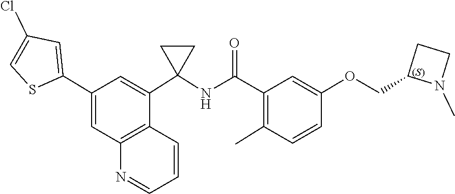 | A |
| 623 | 530 | 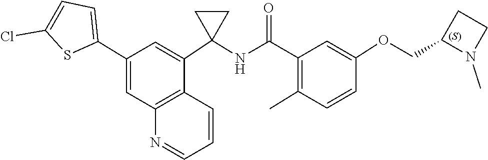 | A |
| 604 | 531 | 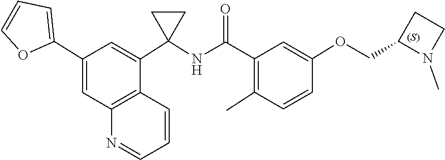 | A |
| 652 | 532 | 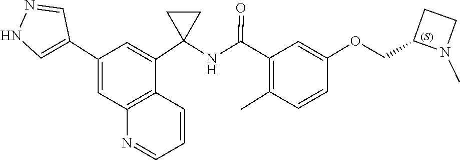 | A |
| 670 | 533 | 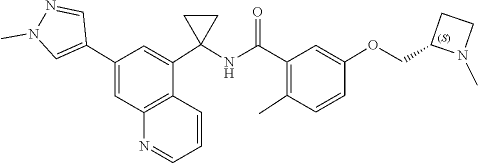 | A |
| 633 | 534 | 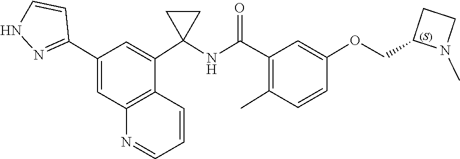 | A |
| 651 | 535 | 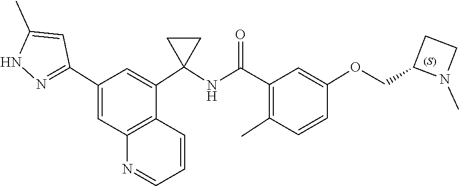 | A |
| 582 | 536 | 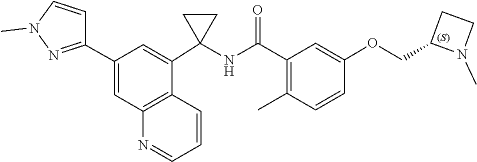 | A |
| 644 | 537 | 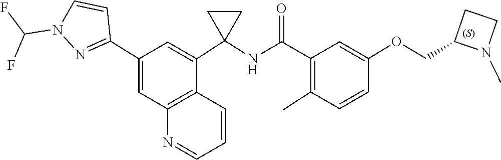 | A |
| 659 | 538 | 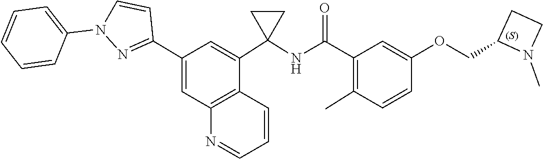 | A |
| 609 | 539 | 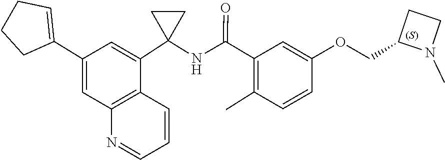 | A |
| 621 | 540 | 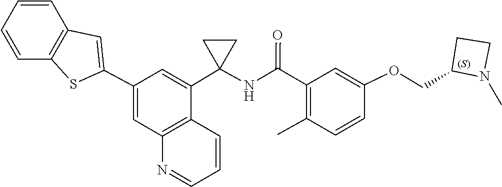 | A |
| 594 | 541 | 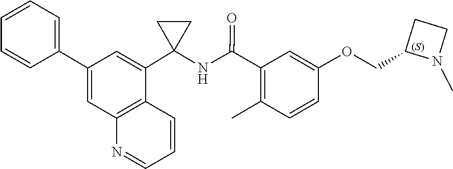 | A |
| 574 | 542 | 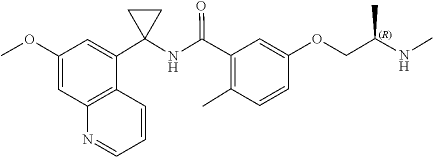 | A |
| 592 | 543 | 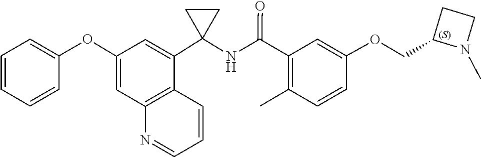 | A |
| 590 | 544 | 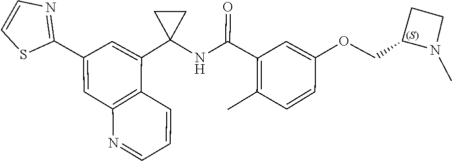 | A |
| 591 | 545 | 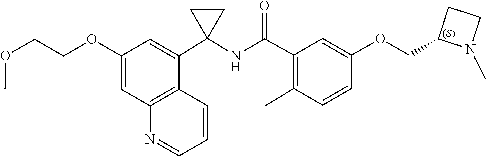 | A |
| 612 | 546 | 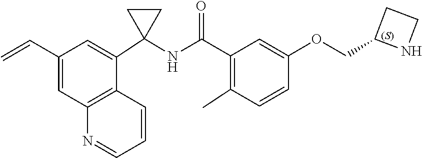 | A |
| 613 | 547 | 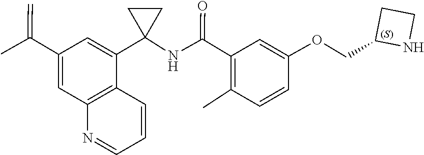 | A |
| 667 | 548 | 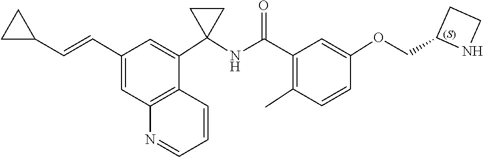 | A |
| 704 | 549 | 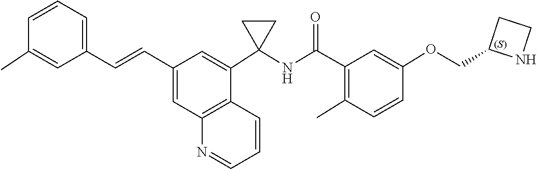 | A |
| 640 | 550 | 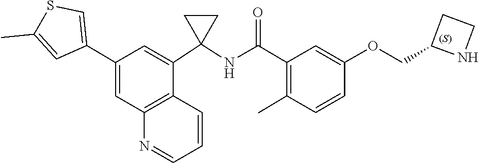 | A |
| 643 | 551 | 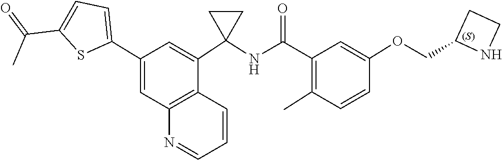 | A |
| 658 | 552 | 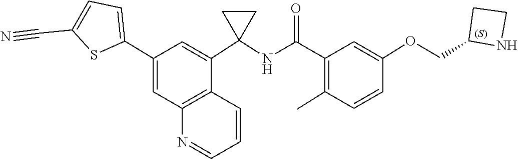 | A |
| 673 | 553 | 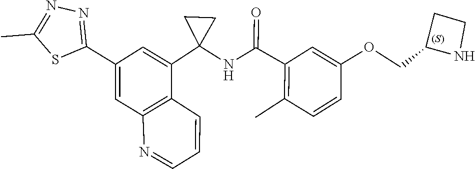 | A |
| 645 | 554 | 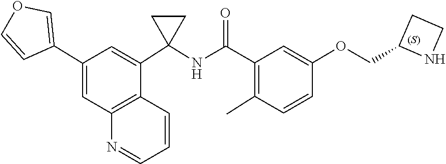 | A |
| 641 | 555 | 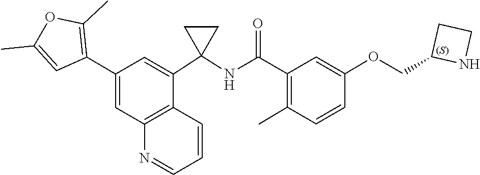 | A |
| 646 | 556 | 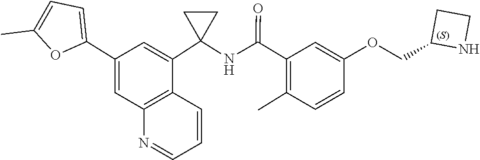 | A |
| 694 | 557 | 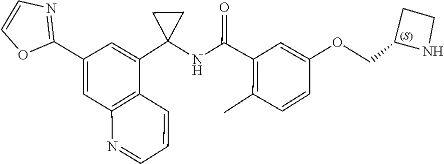 | A |
| 698 | 558 | 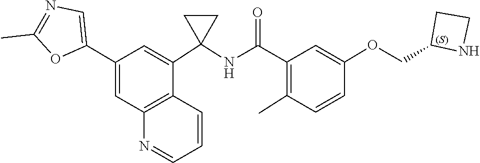 | A |
| 680 | 559 | 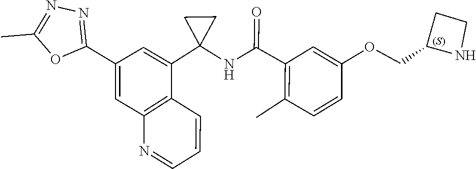 | A |
| 636 | 560 | 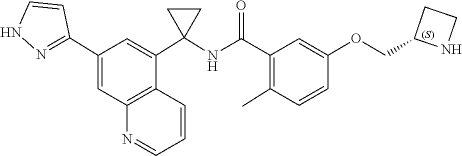 | A |
| 635 | 561 | 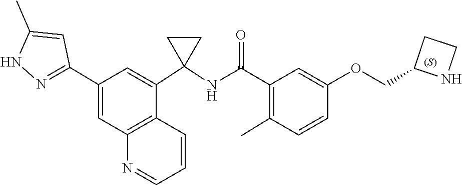 | A |
| 649 | 562 | 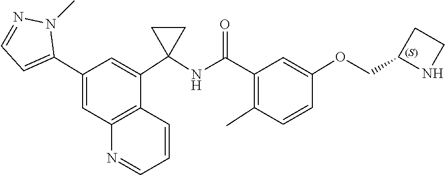 | A |
| 634 | 563 | 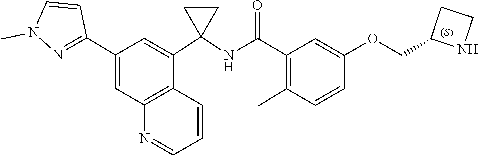 | A |
| 656 | 564 | 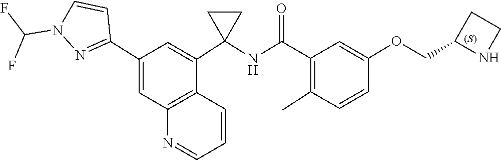 | A |
| 664 | 565 | 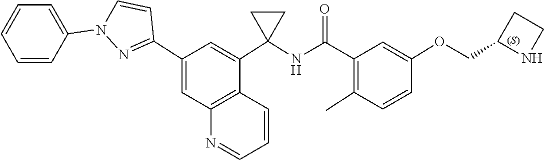 | A |
| 642 | 566 | 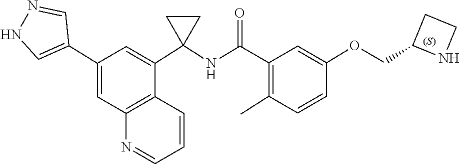 | A |
| 665 | 567 | 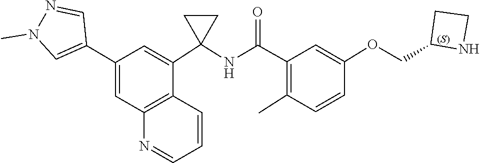 | A |
| 695 | 568 | 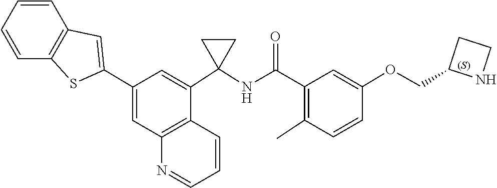 | A |
| 711 | 569 | 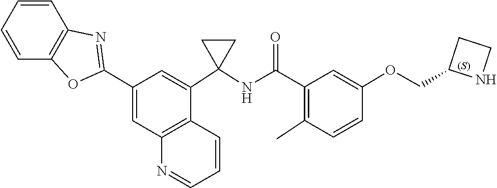 | A |
| 662 | 570 | 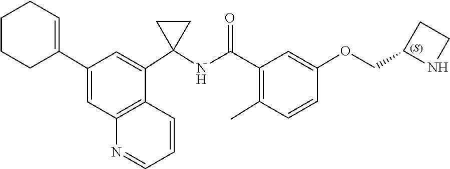 | A |
| 675 | 571 | 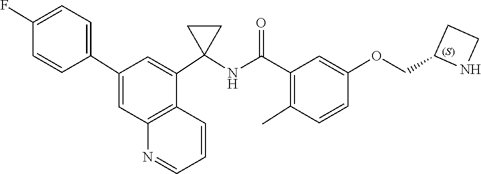 | A |
| 666 | 572 | 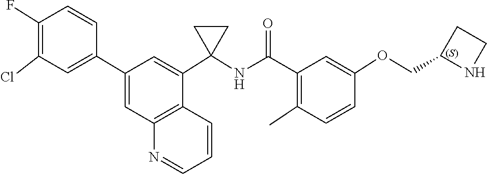 | A |
| 674 | 573 | 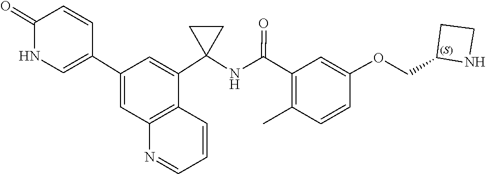 | A |
| 637 | 574 | 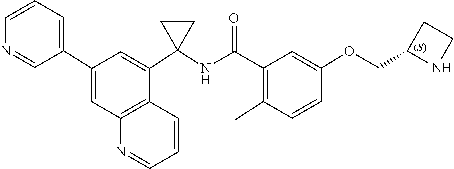 | A |
| 682 | 575 | 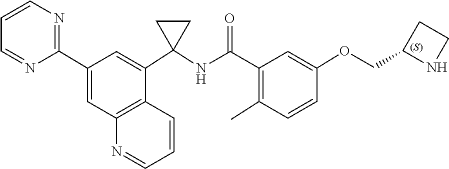 | A |
| 688 | 576 | 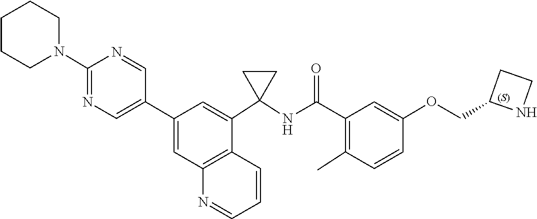 | A |
| 686 | 577 | 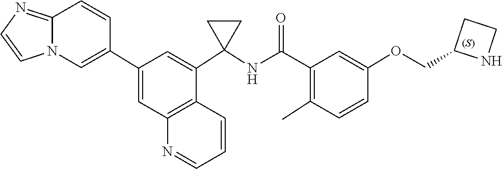 | A |
| 685 | 578 | 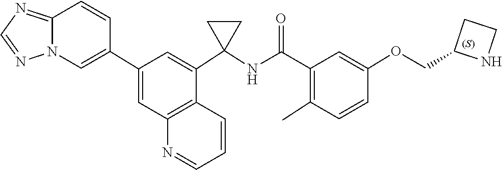 | A |
| 822 | 579 | 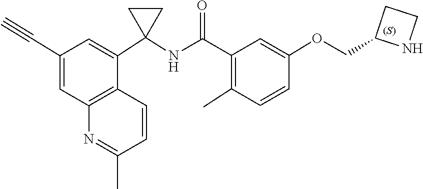 | A |
| 615 | 580 | 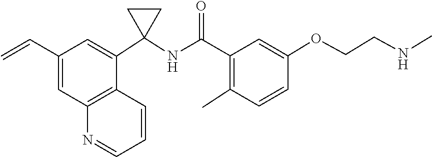 [SMILES: C=Cc1cc(C2(NC(=O)c3cc(OCCNC)ccc3C)CC2)c2cccnc2c1] | A |
| 616 | 581 | 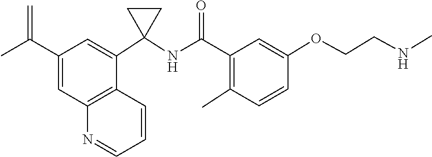 [SMILES: C=C(C)c1cc(C2(NC(=O)c3cc(OCCNC)ccc3C)CC2)c2cccnc2c1] | A |
| 639 | 582 | 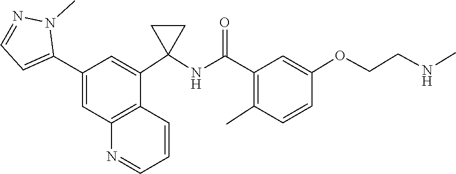 [SMILES: CNCCOc1ccc(C)c(C(=O)NC2(c3cc(-c4ccnn4C)cc4ncccc34)CC2)c1] | A |
| 647 | 583 | 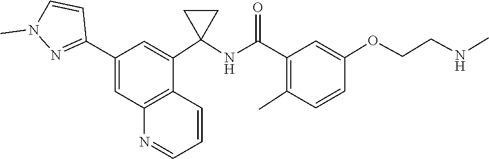 | A |
| 802 | 584 | 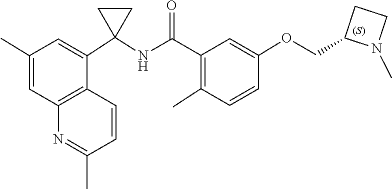 | A |
| 717 | 585 | 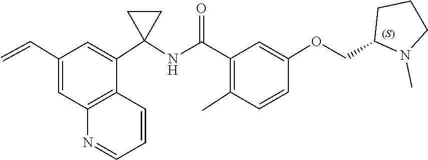 | A |
| 813 | 586 | 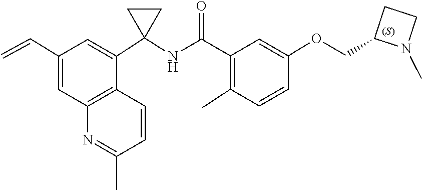 | A |
| 719 | 587 | 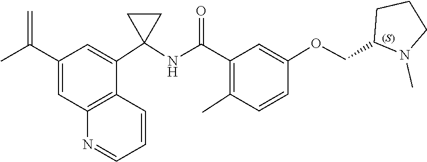 | A |
| 684 | 588 | 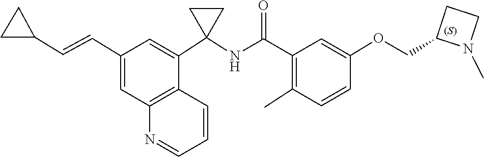 | A |
| 690 | 589 | 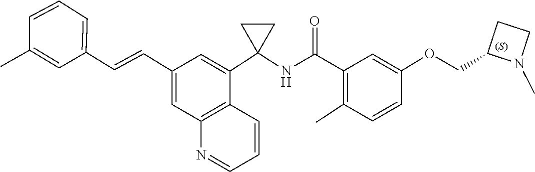 | A |
| 721 | 590 | 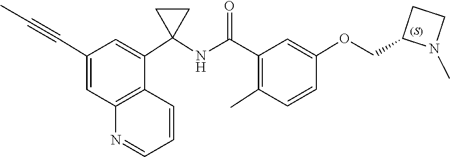 | A |
| 657 | 591 | 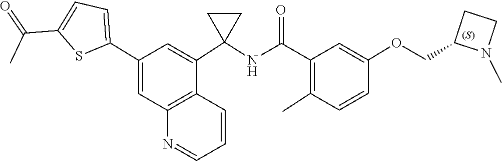 | A |
| 754 | 592 | 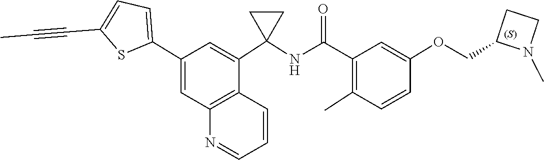 | A |
| 764 | 593 | 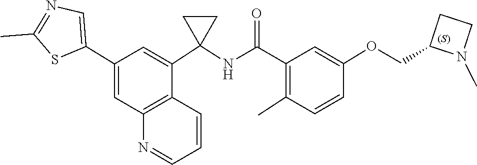 | A |
| 691 | 594 | 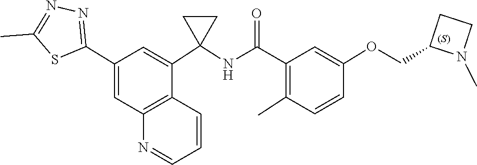 | A |
| 654 | 595 | 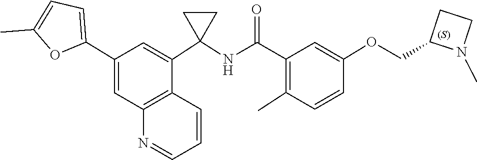 | A |
| 655 | 596 | 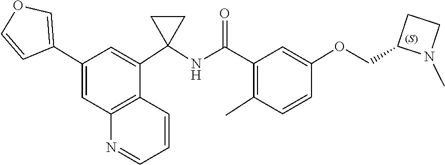 | A |
| 671 | 597 | 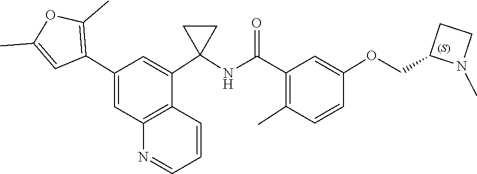 | A |
| 716 | 598 | 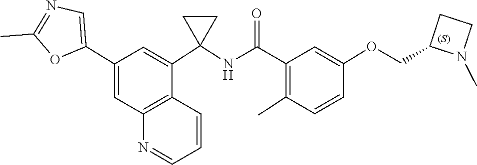 | A |
| 693 | 599 | 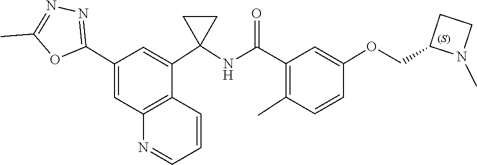 | A |
| 820 | 600 | 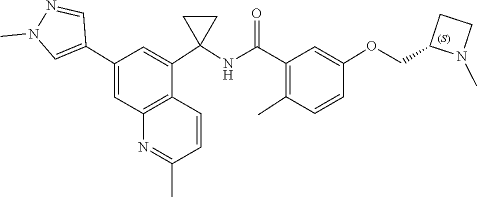 | |
| 718 | 601 | 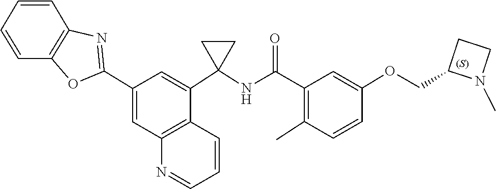 | A |
| 736 | 602 | 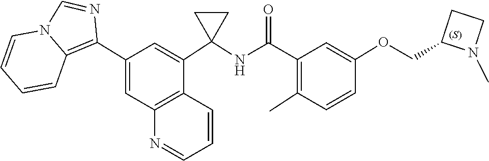 | A |
| 663 | 603 | 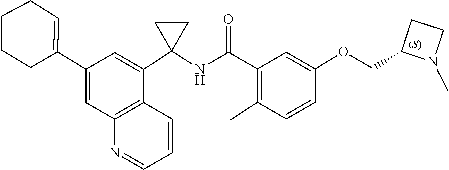 | A |
| 678 | 604 | 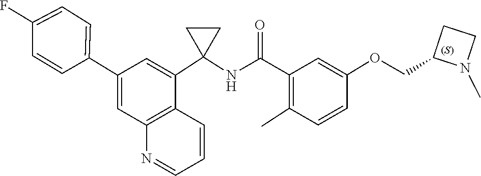 | A |
| 687 | 605 | 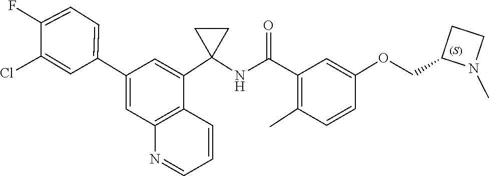 | A |
| 672 | 606 | 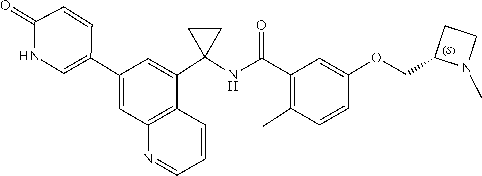 | A |
| 681 | 607 | 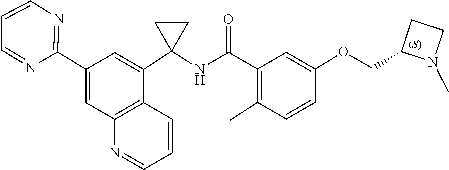 | A |
| 701 | 608 | 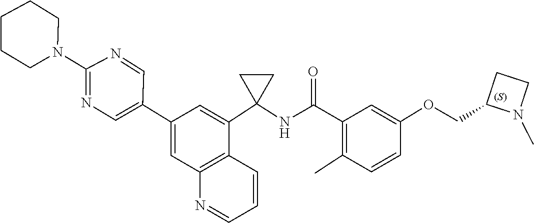 | A |
| 699 | 609 | 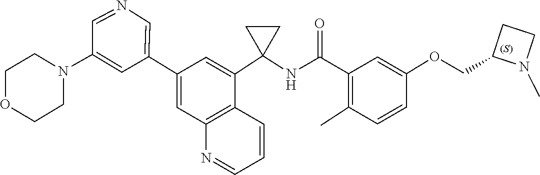 | A |
| 702 | 610 | 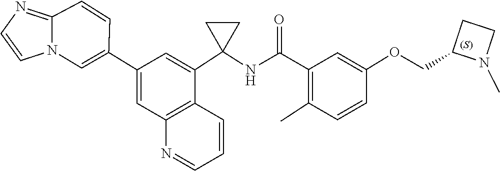 | A |
| 700 | 611 | 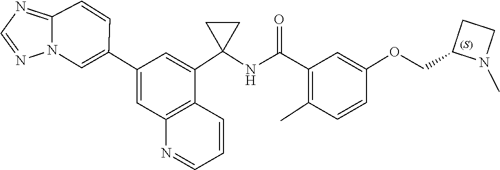 | A |
| 661 | 612 | 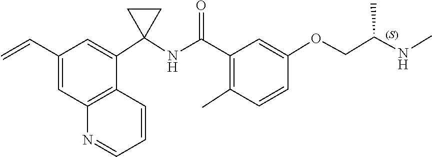 | A |
| 735 | 613 | 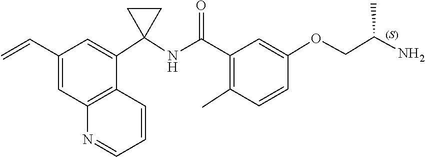 | A |
| 734 | 614 | 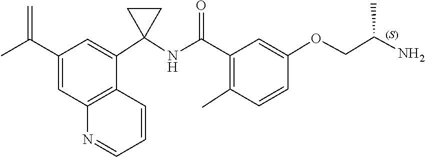 | A |
| 751 | 615 | 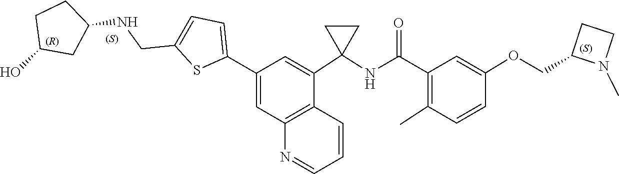 | A |
| 756 | 616 | 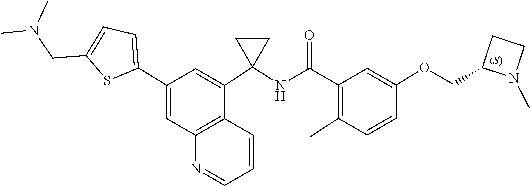 | A |
| 805 | 617 | 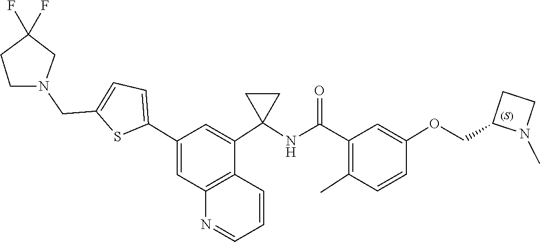 | A |
| 752 | 618 | 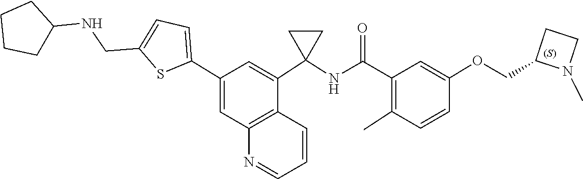 | A |
| 793 | 619 | 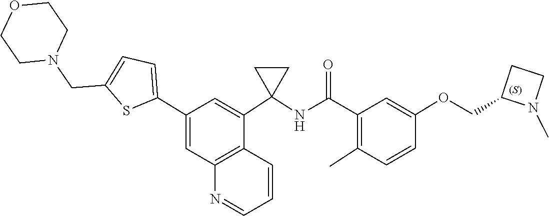 | A |
| 781 | 620 | 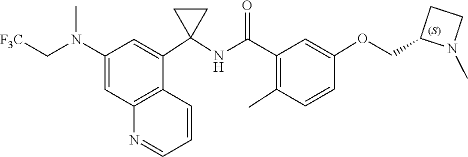 | A |
| 807 | 621 | 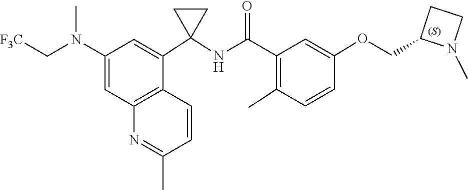 | A |
| 771 | 622 | 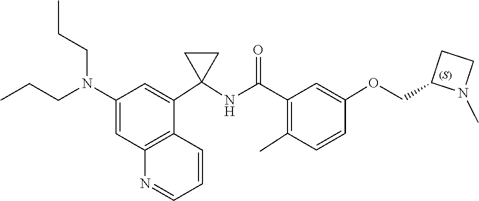 | A |
| 722 | 623 | 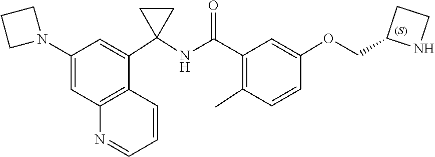 | A |
| 723 | 624 | 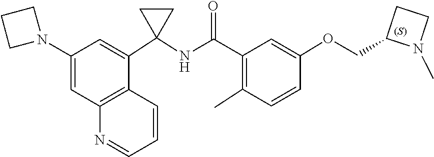 | A |
| 811 | 625 | 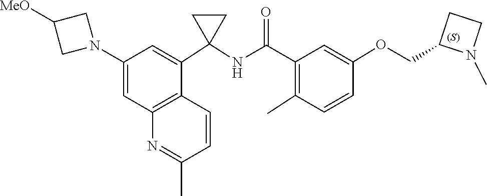 | A |
| 810 | 626 | 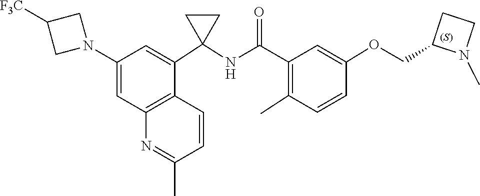 | A |
| 816 | 627 | 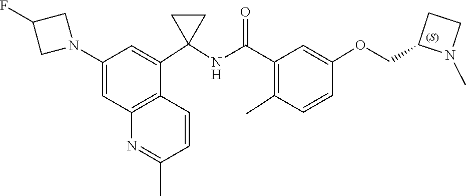 | A |
| 826 | 628 | 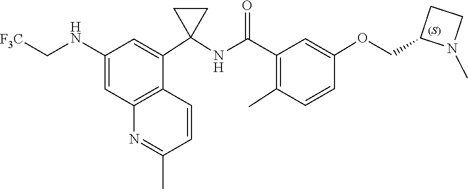 | A |
| 712 | 629 | 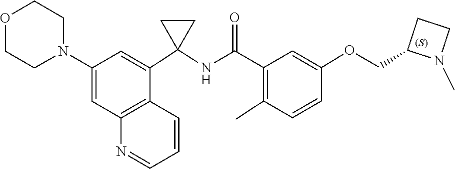 | A |
| 740 | 630 | 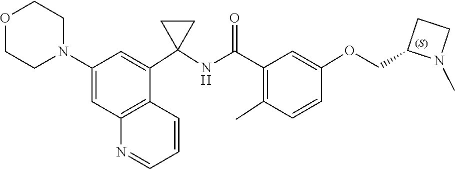 | A |
| 714 | 631 | 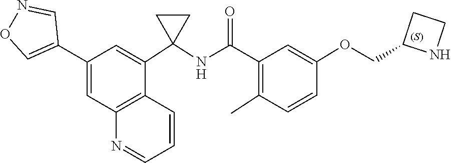 | A |
| 733 | 632 | 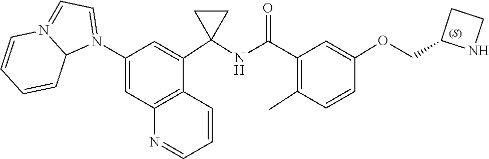 | A |
| 715 | 633 | 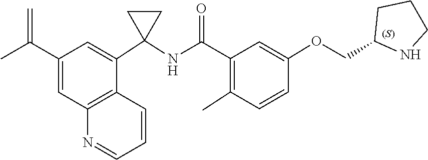 | A |
| 824 | 634 | 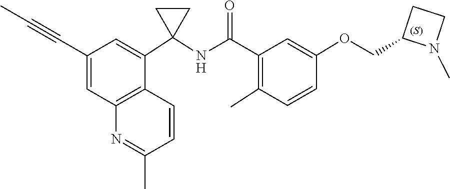 | A |
| 763 | 635 | 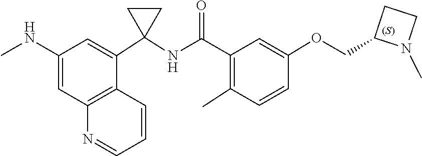 | A |
| 745 | 636 | 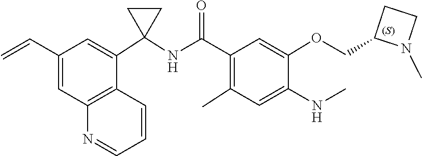 | A |
| 759 | 637 | 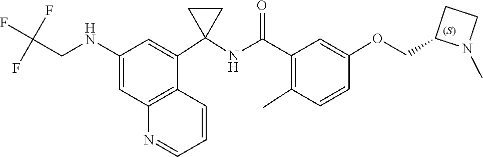 | A |
| 747 | 638 | 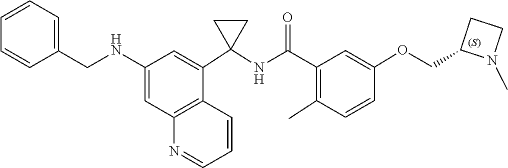 | A |
| 760 | 639 | 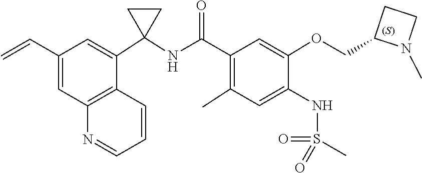 | A |
| 755 | 640 | 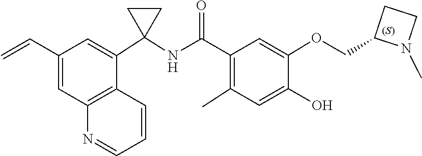 | A |
| 762 | 641 | 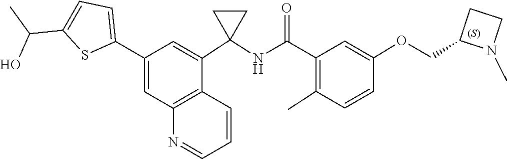 | A |
| 758 | 642 | 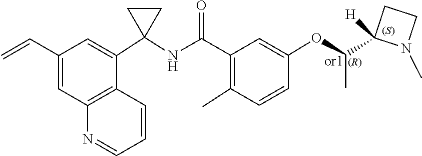 | A |
| 774 | 643 | 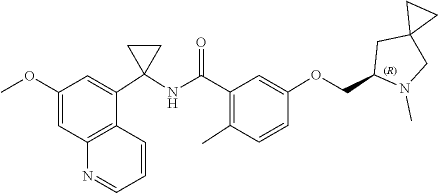 | A |
| 788 | 644 | 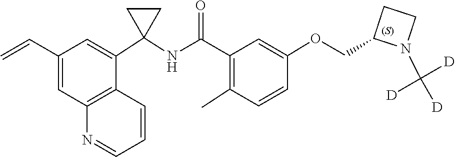 | A |
| 798 | 645 | 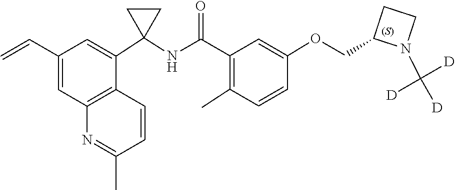 | A |
| 818 | 646 | 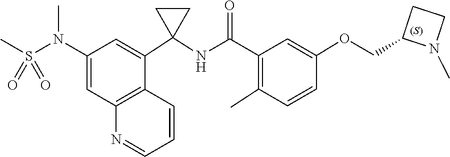 | A |
| 825 | 647 | 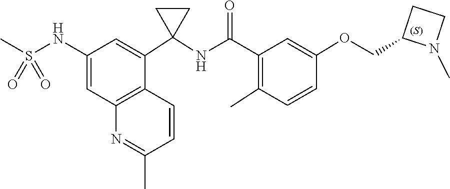 | A |
| 819 | 648 | 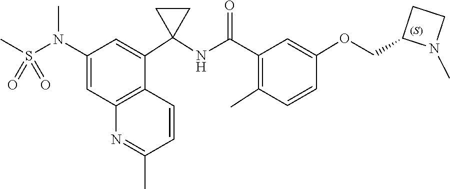 | A |
| 804 | 649 | 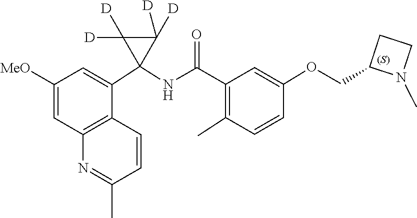 | A |
| 823 | 650 | 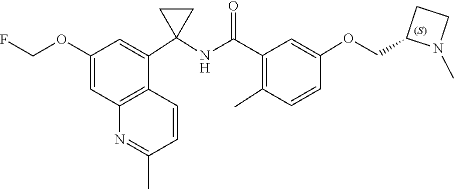 | A |
| 801 | 651 | 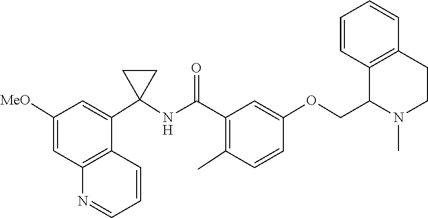 [SMILES: COc1cc(C2(NC(=O)c3cc(OCC4c5ccccc5CCN4C)ccc3C)CC2)c2cccnc2c1] | A |

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A compound of formula (I′), 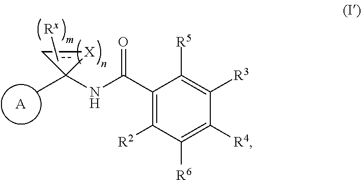
or a pharmaceutically acceptable salt thereof, wherein:
Ring A is selected from the group consisting of naphthyl, anthracenyl, 8-12 membered bicyclic or tricyclic heteroaryl, 8-12 membered bicyclic or tricyclic heterocyclyl, and 8-12 membered partially unsaturated bicyclic carbocyclyl, wherein the naphthyl, 8-12 membered bicyclic or tricyclic heteroaryl, and 8-12 membered partially unsaturated bicyclic carbocyclyl are optionally substituted by one or more (e.g., one, two, three, or four) R¹;
each X is independently selected from the group consisting of CH₂, CH, NR^{h}, and O;
each R^{x} is independently C₁₋₆ alkyl or halo when R^{x} is substituted on a carbon atom or each R^{x} is independently C₁₋₆ alkyl when R^{x} is substituted on a nitrogen atom;
m is 0, 1, or 2; [symbol]
is a single bond or double bond;
n is 1 or 2;
each R¹ is independently selected from the group consisting of phenyl, 5-6 membered heteroaryl, C₃₋₇ carbocyclyl, 3-7 membered heterocyclyl, C₁₋₆ haloalkyl, C₁₋₆ haloalkoxy, halo, —CN, C₁₋₆ alkyl, C₂₋₆ alkenyl, C₂₋₆ alkynyl, C₁₋₆ alkoxy optionally substituted with phenyl, —O—C₁₋₆ alkylene-C₁₋₆ alkoxy, —C(O)ORⁱ, —S(O)ₜ, C₁₋₆alkyl, —S—C₁₋₆ haloalkyl, —NR^{e}R^{f}, —C(O)NR^{g}R^{h}, —O-phenyl, and hydroxy, wherein the phenyl and 5-6 membered heteroaryl are optionally substituted with one or more (e.g., one, two, three, or four) substituents each selected from the group consisting of halo, C₁₋₆ alkyl, C₁₋₆ haloalkyl, and C₁₋₆ alkoxy; wherein t is 0, 1, or 2;
R² is selected from the group consisting of halo, C₁₋₆ alkyl, C₁₋₆ haloalkyl, C₁₋₆ alkoxy, and C₃₋₆ cycloalkyl;
R³ is selected from the group consisting of C₁₋₆ alkoxy, hydroxy, C₀₋₆ alkylene-(3-8 membered heterocyclyl optionally substituted with one or more (e.g., one, two, three, or four) substituents each independently selected from the group consisting of C₁₋₆alkyl, (C₁₋₆alkylene-OH), and —NR^{e}R^{f}), —O—C₃₋₇ cycloalkyl optionally substituted with —NR^{c}R^{d}, —(C₀₋₆alkylene)-O-(3-8 membered heterocyclyl optionally substituted with one or more (e.g., one, two, three, or four) substituents each independently selected from the group consisting of C₁₋₆ alkyl, benzyl and —C(O)O—C₁₋₆ alkyl), C₁₋₆ alkyl optionally substituted with —NR^{c}R^{d}, —O-(5-6 membered heteroaryl), C₀₋₆ alkylene-CN, C₀₋₆ alkylene-C(O)O(C₁₋₆alkyl), C₀₋₆ alkylene-C(O)NR^{e}R^{f}, and —NR^{A}R^{B}, wherein the C₁₋₆ alkoxy is optionally substituted with one or more (e.g., one, two, three, or four) R^{3a},
R⁴ is selected from the group consisting of H, C₁₋₆ alkyl, halo, hydroxy, and —NR^{e}R^{f}; or
R³ and R⁴, together with the atoms to which they are attached, combine to form an aromatic or non-aromatic 5-10 membered monocyclic or bicyclic ring fused to the phenyl, wherein the 5-10 membered ring comprises at least one heteroatom, wherein the 5-10 membered ring is optionally substituted with one or more (e.g., one, two, three, or four) substituents each independently selected from the group consisting of C₁₋₆ alkyl optionally substituted with —NR^{c}R^{d}, C₃₋₇ cycloalkyl, phenyl, 5-6 membered heteroaryl, —(C₀₋₆ alkylene)-C(O)NR^{e}R^{f}, —C(O)—C₁₋₆heteroalkyl, —C(O)—NCH₃OCH₃, —(C₀₋₆ alkylene)-NR^{c}R^{d}, —(C₀₋₆ alkylene)-C₁₋₆ alkoxy, —(C₀₋₆ alkylene)-OH, oxo, —C(O)OH, and —(C₀₋₆ alkylene)-3-7 membered heterocyclyl optionally substituted with C₁₋₆alkyl;
R⁵ is selected from the group consisting of H, halo, and C₁₋₆ alkyl;
R⁶ is selected from the group consisting of H, halo, and C₁₋₆ alkyl;
each R^{3a} is independently selected from the group consisting of C₁₋₆ alkyl, —NR^{A}R^{B}, C₁₋₆ alkoxy, hydroxy, —C(O)NR^{c}R^{d}, 3-8 membered heterocyclyl, phenyl, 5-6 membered heteroaryl, and —(C₀₋₆ alkylene)-(C₃₋₆ cycloalkyl optionally substituted with NR^{c}R^{d}), wherein the 3-8 membered heterocyclyl and 5-6 membered heteroaryl are optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, phenyl, benzyl, C₁₋₆ alkylene-OH, and C₀₋₆ alkylene-C₁₋₆ alkoxy;
each R^{A} and R^{B} are independently, for each occurrence, selected from the group consisting of H, C₁₋₆ alkyl, C₁₋₆ haloalkyl, C₁₋₆ alkoxy, S(O)₂C₁₋₆ alkyl, —C(O)—C₁₋₆ alkyl, C₁₋₆ alkylene-NR^{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy or C₁₋₆ alkoxy;
each R^{c} and R^{d} are independently, for each occurrence, selected from the group consisting of H, C₁₋₆ alkyl, benzyl, and —C(O)OC₁₋₆ alkyl, or
R^{c} and R^{d} can be taken together with the nitrogen atom to which they are attached to form a 3-7 membered heterocyclyl;
each R^{e} and R^{f} are independently, for each occurrence, selected from the group consisting of H, C₁₋₆ alkyl, —C(O) C₁₋₆alkyl, and —C(O)OC₁₋₆alkyl; and
each R^{g}, R^{h}, and Rⁱ are independently, for each occurrence, H or C₁₋₆ alkyl.

2. A compound of formula (I″), 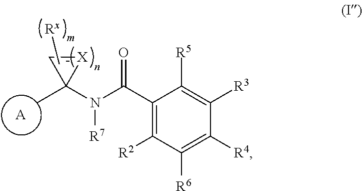
or a pharmaceutically acceptable salt thereof, wherein:
Ring A is selected from the group consisting of naphthyl, anthracenyl, phenanthrenyl, 8-14 membered bicyclic or tricyclic heteroaryl, 8-12 membered bicyclic or tricyclic heterocyclyl, and 8-12 membered partially unsaturated bicyclic carbocyclyl, wherein the naphthyl, 8-14 membered bicyclic or tricyclic heteroaryl, and 8-12 membered partially unsaturated bicyclic carbocyclyl are optionally substituted by one or more (e.g., one, two, three, or four) R¹,
wherein when ring A is 8-12 membered bicyclic heterocyclyl or 8-12 membered partially unsaturated bicyclic carbocyclyl, R³ is C₁₋₆ alkoxy optionally substituted with one or more (e.g., one, two, three, or four) R^{3a};
each X is independently selected from the group consisting of CH₂, CH, NR^{h}, and O;
each R is independently D, C₁₋₆ alkyl or halo when R^{x} is substituted on a carbon atom or each R^{x} is independently C₁₋₆ alkyl when R^{x} is substituted on a nitrogen atom;
m is 0, 1, 2, 3, or 4; [symbol]
is a single bond or double bond;
n is 1 or 2;
each R¹ is independently selected from the group consisting of phenyl, 5-9 membered heteroaryl, C₃₋₇ carbocyclyl, 3-7 membered heterocyclyl optionally substituted with one or more (e.g., one, two, three, or four) R^{1a}, C₁₋₆ haloalkyl, C₁₋₆ haloalkoxy, halo, —CN, C₁₋₆ alkyl optionally substituted with hydroxy, C₂₋₆ alkenyl optionally substituted with C₃₋₆ cycloalkyl or phenyl substituted with C₀₋₆alkyl, C₂₋₆ alkynyl optionally substituted with one or more (e.g., one, two, three, or four) halo, C₁₋₆ alkoxy optionally substituted with phenyl, —O—C₁₋₆ alkylene-C₁₋₆ alkoxy, —C(O)—C₁₋₆ alkyl, —C(O)ORⁱ, —S(O)ₜC₁₋₆alkyl, —S—C₁₋₆ haloalkyl, —OS(O)ₜC₁₋₆haloalkyl, —NR^{j}R^{k}, —C(O)NR^{g}R^{h}, —O-phenyl, —B(OR^{m})₂, and hydroxy, wherein the phenyl and 5-9 membered heteroaryl are optionally substituted with one or more (e.g., one, two, three, or four) substituents each independently selected from the group consisting of halo, cyano, phenyl, 3-7 membered heterocyclyl, C₁₋₆ alkyl, C₁₋₆alkylene-(3-7 membered heterocyclyl optionally substituted with one or more (e.g., one, two, three, or four) halo), C₁₋₆alkylene substituted with hydroxy, C₁₋₆ alkylene-NR^{A}R^{B}, —C(O)-3-6 membered heterocyclyl, hydroxy, C₁₋₆ haloalkyl, C₁₋₆ alkoxy, and —C(O)—C₁₋₆ alkyl; wherein t is 0, 1, or 2;
R^{1a} is selected from the group consisting of oxo, halo, C₁₋₆haloalkyl, and C₁₋₆alkoxy;
R⁵ is selected from the group consisting of H, halo, and C₁₋₆ alkyl;
R⁶ is selected from the group consisting of H, halo, and C₁₋₆ alkyl;
R⁷ is selected from H and C₁₋₆ alkyl;
R² is selected from the group consisting of halo, C₁₋₆ alkyl, C₂₋₆ alkynyl, C₁₋₆ haloalkyl, C₁₋₆ alkoxy, and C₃₋₆ cycloalkyl;
R³ is selected from the group consisting of C₁₋₂₀ alkoxy, hydroxy, C₀₋₆ alkylene-(3-10 membered heterocyclyl optionally substituted with one or more (e.g., one, two, three, or four) substituents each independently selected from the group consisting of C₁₋₆alkyl, (C₁₋₆alkylene-OH), —C(O)O—C₁₋₆ alkyl, and —NR^{e}R^{f}, —O—C₃₋₇ cycloalkyl optionally substituted with —NR^{c}R^{d}, —(C₀₋₆alkylene)-O-(3-9 membered heterocyclyl optionally substituted with one or more (e.g., one, two, three, or four) substituents each independently selected from the group consisting of C₁₋₆ alkyl, benzyl and —C(O)O—C₁₋₆ alkyl), C₁₋₆ alkyl optionally substituted with —NR^{c}R^{d}, —O-(5-6 membered heteroaryl), C₀₋₆ alkylene-CN, C₀₋₆ alkylene-C(O)O(C₁₋₆alkyl), C₀₋₆ alkylene-C(O)NR^{e}R^{f}, and —NR^{AA}R^{BB}, wherein the C₁₋₂₀ alkoxy is optionally substituted with one or more (e.g., one, two, three, or four) R^{3a};
R⁴ is selected from the group consisting of H, C₁₋₆ alkyl, C₂₋₆ alkynyl optionally substituted with phenyl or 5-6 membered heteroaryl, halo, hydroxy, and —NR^{e}R^{f}; or
R³ and R⁴, together with the atoms to which they are attached, combine to form an aromatic or non-aromatic 5-10 membered monocyclic or bicyclic ring fused to the phenyl, wherein the 5-10 membered ring comprises at least one heteroatom, wherein the 5-10 membered ring is optionally substituted with one or more (e.g., one, two, three, or four) substituents each independently selected from the group consisting of C₁₋₆ alkyl optionally substituted with —NR^{c}R^{d}, C₃₋₇ cycloalkyl, phenyl, 5-6 membered heteroaryl, —(C₀₋₆ alkylene)-C(O)NR^{e}R^{f}, —C(O)—C₁₋₆heteroalkyl, —C(O)—NCH₃OCH₃, —(C₀₋₆ alkylene)-NR^{c}R^{d}, —(C₀₋₆ alkylene)-C₁₋₆ alkoxy, —(C₀₋₆ alkylene)-OH, oxo, —C(O)OH, and —(C₀₋₆ alkylene)-3-7 membered heterocyclyl optionally substituted with C₁₋₆alkyl, wherein the aromatic or non-aromatic 5-10 membered monocyclic or bicyclic ring fused to the phenyl is not an imidazole;
R^{AA} and R^{BB} are independently selected from the group consisting of H, C₁₋₆ alkyl, C₁₋₆ haloalkyl, C₁₋₆ alkoxy, S(O)₂C₁₋₆ alkyl, —C(O)—C₁₋₆ alkyl, C₁₋₂₀ alkylene-NR^{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, —(C₀₋₂₀ alkylene)-(3-10 membered heterocyclyl), and —(C₀₋₆ alkylene)-C₃₋₆ cycloalkyl, wherein the C₁₋₆ alkyl, C₁₋₆ alkylene, 3-10 membered heterocyclyl, and C₃₋₆ cycloalkyl are each optionally substituted with one or more (e.g., one, two, three, or four) substituents each independently selected from the group consisting of C₁₋₆ alkyl, hydroxy and C₁₋₆ alkoxy;
each R^{A} and R^{B} are independently, for each occurrence, selected from the group consisting of H, C₁₋₆ alkyl, C₁₋₆ haloalkyl, C₁₋₆ alkoxy, S(O)₂C₁₋₆ alkyl, —C(O)—C₁₋₆ alkyl, C₁₋₆ alkylene-NR^{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy or C₁₋₆ alkoxy;
each R^{c} and R^{d} are independently, for each occurrence, selected from the group consisting of H, C₁₋₆ alkyl, benzyl, and —C(O)OC₁₋₆ alkyl, or
R^{c} and R^{d} can be taken together with the nitrogen atom to which they are attached to form a 3-7 membered heterocyclyl;
each R^{e} and R^{f} are independently, for each occurrence, selected from the group consisting of H, C₁₋₆ alkyl, —C(O) C₁₋₆alkyl, —C(O)OC₁₋₆alkyl, and —S(O)₂C₁₋₆alkyl;
each R^{g}, R^{h}, and Rⁱ are independently, for each occurrence, H or C₁₋₆ alkyl; and
each R^{j} and R^{k} are independently, for each occurrence, selected from the group consisting of H, C₁₋₆ alkyl optionally substituted with phenyl, C₁₋₆ haloalkyl, —C(O) C₁₋₆alkyl, —S(O)₂C₁₋₆alkyl, and —C(O)OC₁₋₆alkyl;
each R^{3a} is independently selected from the group consisting of D, C₁₋₆ alkyl, —NR^{A}R^{B}, C₁₋₆ alkoxy, hydroxy, —C(O)NR^{c}R^{d}, 3-10 membered heterocyclyl, phenyl, 5-6 membered heteroaryl, and —(C₀₋₆ alkylene)-(C₃₋₆ cycloalkyl optionally substituted with NR^{c}R^{d}), wherein the 3-10 membered heterocyclyl and 5-6 membered heteroaryl are optionally substituted with one or more (e.g., one, two, three, or four) substituents each independently selected from the group consisting of halo, hydroxy, C₁₋₆ alkyl optionally substituted with one or more (e.g., one, two, three, or four) D, C₁₋₆ haloalkyl, —C(O)OC₁₋₆ alkyl, C₁₋₆ alkyelene-C₃₋₇ cycloalkyl, phenyl, benzyl, C₁₋₆ alkylene-OH, and C₀₋₆ alkylene-C₁₋₆ alkoxy; and
each R^{m} is independently selected from hydrogen and C₁₋₆alkyl or two (OR^{m}) groups can be taken together with the boron atom to which they are attached to form pinacol ester;
with the proviso that, when ring A is naphthyl, R⁴ is selected from the group consisting of hydrogen, —F, —Cl, C₁₋₆ alkyl, and —NR^{e}R^{f}; R⁵ is hydrogen; R⁶ is hydrogen or halo; X is CH₂; and n is 1;
R² is selected from the group consisting of halo, C₁₋₆ alkyl, C₂₋₆ alkynyl, C₁₋₆ alkoxy, and C₃₋₆ cycloalkyl;
R³ is selected from the group consisting of C₁₋₆ alkoxy, hydroxy, C₁₋₆ alkylene-(3-10 membered heterocyclyl optionally substituted with one or more (e.g., one, two, three, or four) R^{XX}), 3 membered heterocyclyl optionally substituted with one or more (e.g., one, two, three, or four) R^{XX}, 4 membered heterocyclyl substituted with one or more (e.g., one, two, three, or four) R^{XX}, 5 membered partially unsaturated heterocyclyl, 5 membered saturated heterocyclyl that has the point of attachment (to the phenyl) on the carbon atom of the heterocyclyl, 5 membered saturated heterocyclyl substituted with at least 2 (e.g., 2, 3, or 4) of R^{XX}, 6-membered heterocyclyl with at least 2 (e.g., 2, 3, or 4) of R^{XX} substituted on the carbon atoms of the heterocyclyl, —O—C₃₋₇ cycloalkyl optionally substituted with —NR^{c}R^{d}, —(C₀₋₆alkylene)-O-(3-9 membered heterocyclyl optionally substituted with one or more (e.g., one, two, three, or four) substituents each independently selected from the group consisting of C₁₋₆ alkyl, benzyl and —C(O)O-C₁₋₆ alkyl), C₁₋₆ alkyl optionally substituted with —NR^{c}R^{d}, —O-5-6 membered heteroaryl, C₀₋₆ alkylene-CN, C₀₋₆ alkylene-C(O)O(C₁₋₆alkyl), C₀₋₆ alkylene-C(O)NR^{e}R^{f}, and —NR^{AA}R^{BB}, wherein the C₁₋₆ alkoxy is optionally substituted with one or more (e.g., one, two, three, or four) R^{3a};
each R^{XX} is independently selected from the group consisting of C₁₋₆alkyl, (C₁₋₆alkylene)-OH, —C(O)O—C₁₋₆ alkyl, and —NR^{e}R^{f}, and
R^{AA} and R^{BB} are independently selected from the group consisting of H, C₁₋₆ alkyl, C₁₋₆ haloalkyl, C₁₋₆ alkoxy, —C(O)—C₁₋₆ alkyl, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy or C₁₋₆ alkoxy, and at least one of R^{AA} and R^{BB} is not hydrogen; or R^{AA} is hydrogen and R^{BB} is C₁₋₆ alkylene-NR^{c}R^{d}.

3. The compound of claim 1 or 2, wherein the compound is a compound of formula (Ia′): 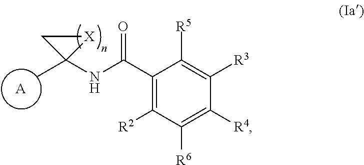
or a pharmaceutically acceptable salt thereof, wherein the variables are as defined in claim 1 or 2.

4. The compound any one of claims 1-3, wherein the compound is a compound of formula (Ib′): 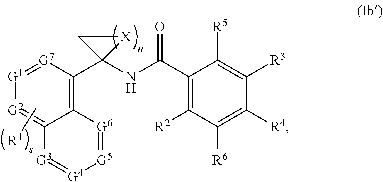
or a pharmaceutically acceptable salt thereof, wherein:
each of G¹, G², G³, G⁴, G⁵, G⁶, and G⁷ is independently selected from CH and N;
s is 0, 1, 2, or 3;
wherein X, n, R¹, R², R³, R⁴, R⁵, and R⁶ are as defined in claim 1 or 2.

5. The compound of any one of claims 1-4, wherein the compound is a compound of formula (Ic′): 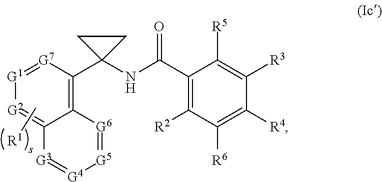
or a pharmaceutically acceptable salt thereof, wherein the variables are as defined in claim 4.

6. The compound of any one of claims 1-5, wherein the compound is a compound of formula (Id′): 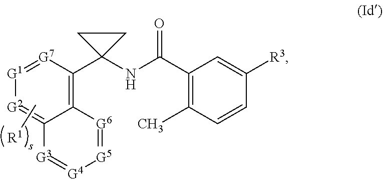
or a pharmaceutically acceptable salt thereof, wherein the variables are as defined in claim 4.

7. The compound of any one of claims 4-6, wherein G¹, G², G³, G⁴, G⁵, G⁶, and G⁷ are CH.

8. The compound of any one of claims 4-6, wherein G¹ is N, and G², G³, G⁴, G⁵, G⁶, and G⁷ are CH; or G² is N, and G¹, G³, G⁴, G⁵, G⁶, and G⁷ are CH.

9. The compound of any one of claims 4-6, wherein G³ is N, and G¹, G², G⁴, G⁵, G⁶, and G⁷ are CH; or G⁴ is N, and G¹, G², G³, G⁵, G⁶, and G⁷ are CH.

10. The compound of any one of claims 4-6, wherein G⁵ is N, and G¹, G², G³, G⁴, G⁶, and G⁷ are CH; or G⁶ is N, and G¹, G², G³, G⁴, G⁵, and G⁷ are CH.

11. The compound of any one of claims 4-6, wherein G⁷ is N, and G¹, G², G³, G⁴, G⁵, and G⁶ are CH.

12. The compound of any one of claims 4-6, wherein s is 0.

13. The compound of any one of claims 4-6, wherein s is 1.

14. The compound of any one of claims 4-6, wherein s is 2.

15. The compound of any one of claims 1-3, wherein Ring A is naphthyl.

16. The compound of claim 15, wherein Ring A is selected from

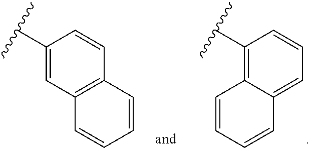 [SMILES: *c1ccc2ccccc2c1 ; *c1cccc2ccccc12]

17. The compound of claim 2, wherein Ring A is anthracenyl.

18. The compound claim 2, wherein Ring A is phenanthrenyl.

19. The compound of any one of any one of claims 1-3, wherein Ring A is 8-14 membered bicyclic or tricyclic heteroaryl.

20. The compound of any one of claims 1-3 and 19, wherein Ring A is 10-membered bicyclic heteroaryl.

21. The compound of any one of claims 1-3, 19, and 20, wherein Ring A is quinolinyl.

22. The compound of claim 21, wherein Ring A is selected from the group consisting of

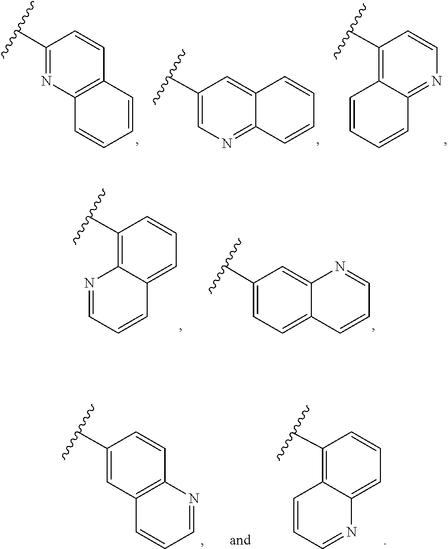

23. The compound of claim 22, wherein Ring A is

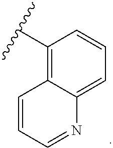 [SMILES: *c1cccc2ncccc12]

24. The compound of any one of claims 1-3, 19, and 20, wherein Ring A is isoquinolinyl,

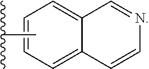

25. The compound of claim 24, wherein Ring A is selected from the group consisting of

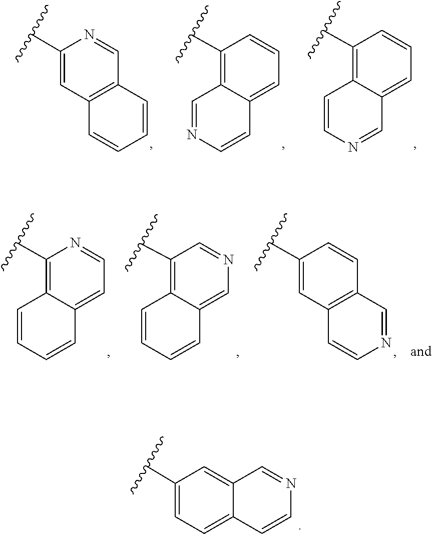

26. The compound of claim 2, wherein the compound is a compound of formula (Ie′): 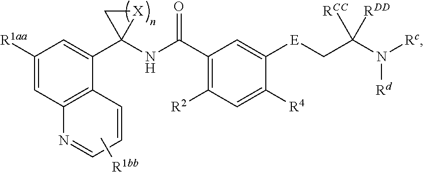
or a pharmaceutically acceptable salt thereof, wherein: 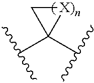
represents cyclopropylene or oxetan-3-ylene (i.e., 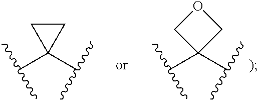 [SMILES: *C1(*)CC1 ; *C1(*)COC1]
R^{1aa} is selected from the group consisting of halo, C₁₋₆ alkyl, C₂₋₆ alkenyl, C₂₋₆ alkynyl, C₁₋₆ alkoxy, C₁₋₆ haloalkyl, —NH(C₁₋₆ alkyl), —N(C₁₋₆ alkyl)₂, 5- or 6-membered monocyclic heteroaryl, 4- or 5-membered heterocyclyl;
R^{1bb} is selected from the group consisting of H, halo, C₁₋₆ alkyl, C₁₋₆ alkoxy, and C₁₋₆ haloalkyl;
R² is selected from the group consisting of halo, C₁₋₆ alkyl, and C₁₋₆ haloalkyl;
R⁴ is selected from the group consisting of H, hydroxy, —NH₂, —NH(C₁₋₆ alkyl), —N(C₁₋₆ alkyl)₂, C₁₋₆ alkyl, C₁₋₆ alkoxy, and C₁₋₆ haloalkyl;
E is selected from the group consisting of —O—, —N(H)—, and —N(C₁₋₆ alkyl)-;
R^{CC} is H or C₁₋₆ alkyl;
R^{DD} is H or C₁₋₆ alkyl;
R^{c} is H or C₁₋₆ alkyl; or
R^{CC} is H, and R^{DD} and R^{c} can be taken together with the carbon and nitrogen atoms to which they are attached to form a 4- to 5-membered heterocycle; and
R^{d} is H or C₁₋₆ alkyl.

27. The compound of claim 26, wherein 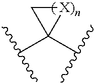
is cyclopropylene (i.e.,

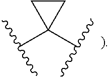 [SMILES: *C1(*)CC1]

28. The compound of claim 26, wherein 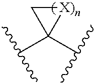
represents oxetan-3-ylene (i.e.,

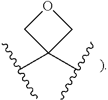 [SMILES: *C1(*)COC1]

29. The compound of any one of claims 26-28, wherein R^{1aa} is C₁₋₆ alkoxy.

30. The compound of any one of claims 26-28, wherein R^{1aa} is C₂₋₆ alkenyl.

31. The compound of any one of claim 26-30, wherein R^{1bb} is H.

32. The compound of any one of claim 26-30, wherein R^{1bb} is C₁₋₆ alkyl.

33. The compound of any one of claims 26-32, wherein R² is C₁₋₆ alkyl.

34. The compound of any one of claims 26-33, wherein E is —O—.

35. The compound of any one of claims 26-33, wherein E is —N(H)—.

36. The compound of any one of claims 26-33, wherein E is —N(C₁₋₆ alkyl)-.

37. The compound of any one of claims 26-36, wherein R^{CC} is H, and R^{DD} and R^{c} are taken together with the carbon and nitrogen atoms to which they are attached to form a 4- to 5-membered heterocycle.

38. The compound of any one of claims 26-37, wherein R^{d} is H.

39. The compound of any one of claims 26-37, wherein R^{d} is C₁₋₆ alkyl.

40. The compound of any one of claims 1-4 and 7-25, wherein X is CH₂.

41. The compound of any one of claims 1, 2, or 7-25, wherein X is CH.

42. The compound of any one of claims 1-4 or 7-25, wherein X is NH.

43. The compound of any one of claims 1-4 and 7-25, wherein X is O.

44. The compound of any one of claims 1-4 and 7-25, wherein n is 2 and one of X is O and the other X is CH₂.

45. The compound of any one of claims 1, 2, 7-25, and 40-44 wherein m is 0.

46. The compound of any one of claims 1, 2, 7-25, and 40-44, wherein m is 1.

47. The compound of any one of claims 1, 2, 7-25, and 40-44, wherein m is 2.

48. The compound of any one of claims 2, 7-25, and 40-44, wherein m is 3.

49. The compound of any one of claims 2, 7-25, and 40-44, wherein m is 4.

50. The compound of any one of claims 1, 2, 7-25, and 40-47, wherein [symbol] is a single bond.

51. The compound of any one of claims 1, 2, 7-25, and 40-47, wherein [symbol] is a double bond.

52. The compound of any one of claims 1-4, 7-25, 40-43, and 45-51, wherein n is 1.

53. The compound of any one of claims 1-4, 7-25, 40-43, and 45-51, wherein n is 2.

54. The compound of any one of the claims 1, 3-6, 13, 14, 19, 20, and 40-53, wherein each R¹ is independently selected from the group consisting of phenyl, 5-6 membered heteroaryl, C₃₋₇ carbocyclyl, C₁₋₆ haloalkyl, halo, —CN, C₁₋₆ alkyl, C₂₋₆ alkenyl, C₁₋₆ alkoxy, —S(O)—C₁₋₆alkyl, —NR^{e}R^{f}, and hydroxy.

55. The compound of any one of the claims 2-6, 13, 14, 19, 20, and 40-53, wherein each R¹ is independently selected from the group consisting of phenyl, 5-9 membered heteroaryl, C₃₋₇ carbocyclyl, 3-7 membered heterocyclyl optionally substituted with one or more (e.g., one, two, three, or four) R^{1a}, C₁₋₆ haloalkyl, halo, —CN, C₁₋₆ alkyl optionally substituted with hydroxy, C₂₋₆ alkenyl optionally substituted with C₃₋₆ cycloalkyl or phenyl substituted with C₀₋₆alkyl, C₂₋₆ alkynyl; optionally substituted with one or more (e.g., one, two, three, or four) halo, C₁₋₆ alkoxy optionally substituted with phenyl, —S(O)ₜ—C₁₋₆alkyl, —C(O)—C₁₋₆ alkyl, OS(O)ₜC₁₋₆haloalkyl, —NR^{j}R^{k} and hydroxy.

56. The compound of any one of claims 1, 3-6, 13, 14, 19, 20, and 40-53, wherein each R¹ is independently selected from the group consisting of phenyl, 5-6 membered heteroaryl, C₃₋₇ carbocyclyl, C₁₋₆ haloalkyl, halo, and C₁₋₆ alkyl, wherein the phenyl and 5-6 membered heteroaryl are optionally substituted with one or more (e.g., one, two, three, or four) substituents each independently selected from the group consisting of halo, C₁₋₆ alkyl, C₁₋₆ haloalkyl, and C₁₋₆ alkoxy.

57. The compound of any one of claims 2-6, 13, 14, 19, 20, and 40-53, wherein each R¹ is independently selected from the group consisting of phenyl, 5-9 membered heteroaryl, C₃₋₇ carbocyclyl, C₁₋₆ haloalkyl, halo, and C₁₋₆ alkyl, wherein the phenyl and 5-9 membered heteroaryl are optionally substituted with one or more (e.g., one, two, three, or four) substituents each independently selected from the group consisting of halo, C₁₋₆ alkyl, C₁₋₆ haloalkyl, and C₁₋₆ alkoxy.

58. The compound of any one of claims 2-6, 13, 14, 19, 20, and 40-53, wherein each R¹ is independently selected from the group consisting of 5-9 membered heteroaryl, 3-7 membered heterocyclyl optionally substituted with one or more (e.g., one, two, three, or four) R^{1a}, C₁₋₆ haloalkyl, halo, C₁₋₆ alkyl optionally substituted with hydroxy, C₂₋₆ alkenyl optionally substituted with C₃₋₆ cycloalkyl or phenyl substituted with C₀₋₆alkyl, C₂₋₆ alkynyl optionally substituted with one or more (e.g., one, two, three, or four) halo, C₁₋₆ alkoxy optionally substituted with phenyl, —NR^{j}R^{k}, wherein the 5-9 membered heteroaryl is optionally substituted with one or more (e.g., one, two, three, or four) substituents each selected from the group consisting of halo, cyano, phenyl, 3-7 membered heterocyclyl, C₁₋₆ alkyl, C₁₋₆alkylene-(3-7 membered heterocyclyl optionally substituted with one or more (e.g., one, two, three, or four) halo), C₁₋₆alkylene substituted with hydroxy, C₁₋₆ alkylene-NR^{A}R^{B}, —C(O)-3-6 membered heterocyclyl, hydroxy, C₁₋₆ haloalkyl, C₁₋₆ alkoxy, and —C(O)—C₁₋₆ alkyl.

59. The compound of any one of claims 2-6, 13, 14, 19, 20, and 40-53, wherein each R¹ is independently selected from the group consisting of 5-9 membered heteroaryl, 3-7 membered heterocyclyl, C₁₋₆ haloalkyl, halo, C₁₋₆ alkyl, C₂₋₆ alkenyl, C₂₋₆ alkynyl, C₁₋₆ alkoxy, and —NR^{j}R^{k}.

60. The compound of any one of claims 2-6, 13, 14, 19, 20, and 40-53, wherein each R¹ is selected from the group consisting of halo, C₁₋₆ alkyl, C₂₋₆ alkenyl, C₂₋₆ alkynyl, C₁₋₆ alkoxy, C₁₋₆ haloalkyl, —NH(C₁₋₆ alkyl), —N(C₁₋₆ alkyl)₂, 5- or 6-membered monocyclic heteroaryl, and 4- or 5-membered heterocyclyl.

61. The compound of any one of claims 2-6, 13, 14, 19, 20, and 40-53, wherein each R¹ is selected from the group consisting of H, halo, C₁₋₆ alkyl, C₁₋₆ alkoxy, and C₁₋₆ haloalkyl.

62. The compound of any one of claims 1-5, 7-25, and 40-61, wherein R² is selected from the group consisting of halo, C₁₋₆ alkyl, C₁₋₆ alkoxy, and C₃₋₆ cycloalkyl.

63. The compound of any one of claims 2-5, 7-25, and 40-61, wherein R² is selected from the group consisting of halo, C₁₋₆ alkyl, C₁₋₆ alkoxy, C₂₋₆ alkynyl, and C₃₋₆ cycloalkyl.

64. The compound of any one of claims 2-5, 7-25, and 40-61, wherein R² is selected from the group consisting of halo, C₁₋₆ alkyl, and C₁₋₆ haloalkyl.

65. The compound of claim 64, wherein R² is halo.

66. The compound of claim 64, wherein R² is C₁₋₆ alkyl.

67. The compound of claim 64, wherein R² is C₁₋₆ haloalkyl.

68. The compound of any one of claims 1, 3-25 and 40-67, wherein R³ is selected from the group consisting of C₁₋₆ alkoxy, hydroxy, C₀₋₆ alkylene-(3-8 membered heterocyclyl optionally substituted with one or more (e.g., one, two, three, or four) substituents each independently selected from the group consisting of C₁₋₆alkyl, (C₁₋₆alkylene-OH), and —NR^{e}R^{f}), —O—C₃₋₇ cycloalkyl optionally substituted with —NR^{c}R^{d}, —(C₀₋₆alkylene)-(O-3-8 membered heterocyclyl optionally substituted with one or more (e.g., one, two, three, or four) substituents each independently selected from the group consisting of C₁₋₆ alkyl benzyl, and —C(O)O—C₁₋₆ alkyl), and —NR^{A}R^{B}, wherein the C₁₋₆ alkoxy is optionally substituted with one or more (e.g., one, two, three, or four) R^{3a}.

69. The compound of any one of claims 2-25 and 40-67, wherein R³ is selected from the group consisting of C₁₋₆ alkoxy, hydroxy, C₀₋₆ alkylene-(3-10 membered heterocyclyl optionally substituted with one or more (e.g., one, two, three, or four) substituents each independently selected from the group consisting of C₁₋₆alkyl, (C₁₋₆alkylene-OH), —C(O)O—C₁₋₆ alkyl, and —NR^{e}R^{f}, —O—C₃₋₇ cycloalkyl optionally substituted with —NR^{c}R^{d}, —(C₀₋₆alkylene)-(O-3-9 membered heterocyclyl optionally substituted with one or more (e.g., one, two, three, or four) substituents each independently selected from the group consisting of C₁₋₆ alkyl benzyl, and —C(O)O—C₁₋₆ alkyl), and NR^{AA}R^{BB}, wherein the C₁₋₆ alkoxy is optionally substituted with one or more (e.g., one, two, three, or four) R^{3a}.

70. The compound of any one of claims 1, 3-25 and 40-67, wherein R³ is selected from the group consisting of C₁₋₆ alkoxy and —(C₀₋₆alkylene)-O-(3-8 membered heterocyclyl optionally substituted with one or more (e.g., one, two, three, or four) substituents each independently selected from the group consisting of C₁₋₆ alkyl, benzyl and —C(O)O—C₁₋₆ alkyl), —O—C₃₋₇ cycloalkyl optionally substituted with —NR^{c}R^{d}, wherein the C₁₋₆ alkoxy is optionally substituted with one or more (e.g., one, two, three, or four) R^{3a}.

71. The compound of any one of claims 1, 3-25 and 40-67, wherein R³ is selected from the group consisting of C₁₋₆ alkoxy and —(C₀₋₆alkylene)-O-(3-9 membered heterocyclyl optionally substituted with one or more (e.g., one, two, three, or four) substituents each independently selected from the group consisting of C₁₋₆ alkyl, benzyl and —C(O)O—C₁₋₆ alkyl), —O—C₃₋₇ cycloalkyl optionally substituted with —NR^{c}R^{d}, wherein the C₁₋₆ alkoxy is optionally substituted with one or more (e.g., one, two, three, or four) R^{3a}.

72. The compound of any one of claims 2-25 and 40-67, wherein R³ is —NR^{AA}R^{BB} or C₁₋₂₀ alkoxy optionally substituted with one or more (e.g., one, two, three, or four) R^{3a}.

73. The compound of any one of claims 2-25 and 40-67 wherein R₃ is C₁₋₂₀ alkoxy optionally substituted with one or more (e.g., one, two, three, or four) R^{3a}.

74. The compound of claim 1 or 68, wherein R^{A} and R^{B} are each independently H or C₁₋₆ alkyl.

75. The compound of any one of claims 2-25 and 40-67, wherein R³ is —NR^{AA}R^{BB}.

76. The compound of any one of claims 2, 69, and 75, wherein R^{AA} and R^{BB} are independently selected from the group consisting of H, C₁₋₆ alkyl, C₁₋₂₀ alkylene-NR^{c}R^{d}, and —(C₀₋₂₀ alkylene)-(3-10 membered heterocyclyl), wherein the 3-10 membered heterocyclyl is optionally substituted with one or more (e.g., one, two, three, or four) substituents each independently selected from the group consisting of C₁₋₆ alkyl, hydroxy, and C₁₋₆ alkoxy.

77. The compound of any one of claims 2, 69, and 75, wherein R^{AA} and R^{BB} are independently selected from the group consisting of H, C₁₋₆ alkyl, C₁₋₂₀ alkylene-NR^{c}R^{d}, and —(C₁₋₂₀ alkylene)-(3-5 membered heterocyclyl), wherein the 3-5 membered heterocyclyl is optionally substituted with C₁₋₆ alkyl.

78. The compound of any one of claim 2, 68-71, 76, or 77, wherein each R^{e} and R^{d} are independently, H or C₁₋₆ alkyl.

79. The compound of any one of claims 1-25 and 40-67, wherein R³ is C₁₋₆ alkoxy optionally substituted with one or more (e.g., one, two, three, or four) R^{3a}.

80. The compound of any one of claims 1-25 and 40-67, wherein R³ is —(C₀₋₆alkylene)-O-(3-8 membered heterocyclyl optionally substituted with one or more (e.g., one, two, three, or four) substituents each independently selected from the group consisting of C₁₋₆ alkyl, benzyl and —C(O)O—C₁₋₆ alkyl).

81. The compound of any one of claims 2-25 and 40-67, wherein R³ is —(C₀₋₆alkylene)-O-(3-9 membered heterocyclyl optionally substituted with one or more (e.g., one, two, three, or four) substituents each independently selected from the group consisting of C₁₋₆ alkyl, benzyl and —C(O)O—C₁₋₆ alkyl).

82. The compound of any one of claims 1-5, 7-25, and 40-81, wherein R⁴ is selected from the group consisting of H, C₁₋₆ alkyl, and halo.

83. The compound of any one of claims 2-5, 7-25, and 40-81, wherein R⁴ is selected from the group consisting of H, C₁₋₆ alkyl, C₂₋₆ alkynyl optionally substituted with phenyl or 5-6 membered heteroaryl, and halo.

84. The compound of any one of claims 2-5, 7-25, and 40-81, wherein R⁴ is selected from the group consisting of H, hydroxy, —NR^{e}R^{f}, C₁₋₆ alkyl, C₁₋₆ alkoxy, and C₁₋₆ haloalkyl.

85. The compound of any one of claims 2-5, 7-25, and 40-81, wherein R⁴ is selected from the group consisting of H, hydroxy, —NH₂, —NH(C₁₋₆ alkyl), —N(C₁₋₆ alkyl)₂, C₁₋₆ alkyl, C₁₋₆ alkoxy, and C₁₋₆ haloalkyl.

86. The compound of any one of claims 1-5, 7-25, and 40-81, wherein R⁴ is H.

87. The compound of any one of claims 1, 3-25, and 40-67, wherein R³ and R⁴, together with the atoms to which they are attached, combine to form an aromatic or non-aromatic 5-10 membered monocyclic or bicyclic ring fused to the phenyl, wherein the 5-10 membered ring comprises at least one heteroatom, wherein the 5-10 membered ring is optionally substituted with one or more (e.g., one, two, three, or four) substituents each independently selected from the group consisting of C₁₋₆ alkyl, C₃₋₇ cycloalkyl, phenyl, 5-6 membered heteroaryl, —(C₀₋₆ alkylene)-C(O)NR^{e}R^{f}, —C(O)—C₁₋₆heteroalkyl, —(C₀₋₆ alkylene)-NR^{c}R^{d}, —(C₀₋₆ alkylene)-C₁₋₆ alkoxy, —(C₀₋₆ alkylene)-OH, oxo, —C(O)OH, and —(C₀₋₆ alkylene)-3-7 membered heterocyclyl optionally substituted with C₁₋₆alkyl.

88. The compound of any one of claims 1-25 and 40-87, wherein R₅ is H or C₁₋₆ alkyl.

89. The compound of claim 88, wherein R⁵ is H.

90. The compound of claim 88, wherein R⁵ is C₁₋₆ alkyl.

91. The compound of any one of claim 1-25 or 40-90, wherein R⁶ is H or C₁₋₆ alkyl.

92. The compound of claim 91, wherein R⁶ is H.

93. The compound of claim 91, wherein R⁶ is C₁₋₆ alkyl.

94. The compound of any one of claims 1-25, 40-73, 79, 82-86, and 88-93, wherein each R^{3a} is independently selected from the group consisting of —NR^{A}R^{B}, C₁₋₆ alkoxy, hydroxy, 3-8 membered heterocyclyl, and —(C₀₋₆ alkylene)-(C₃₋₆ cycloalkyl optionally substituted with NR^{c}R^{d}), wherein the 3-8 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, benzyl, C₁₋₆ alkylene-OH, and C₁₋₆ alkylene-C₁₋₆ alkoxy.

95. The compound of any one of claims 2-25, 40-73, 79, 82-86, and 88-93, wherein each R^{3a} is independently selected from the group consisting of D, —NR^{A}R^{B}, C₁₋₆ alkoxy, hydroxy, 3-10 membered heterocyclyl, and —(C₀₋₆ alkylene)-(C₃₋₆ cycloalkyl optionally substituted with NR^{c}R^{d}), wherein the 3-10 membered heterocyclyl is optionally substituted with one or more (e.g., one, two, three, or four) substituents each independently selected from the group consisting of halo, hydroxy, C₁₋₆ alkyl optionally substituted with one or more (e.g., one, two, three, or four) D, C₁₋₆ haloalkyl, —C(O)OC₁₋₆ alkyl, C₁₋₆ alkyelene-C₃₋₇ cycloalkyl, benzyl, C₁₋₆ alkylene-OH, and C₁₋₆ alkylene-C₁₋₆ alkoxy.

96. The compound of any one of claims 1-25, 40-73, 79, 82-86, and 88-93, wherein each R^{3a} is —NR^{A}R^{B}.

97. The compound of any one of claims 1-25, 40-73, 79, 82-86, and 88-93, wherein each R^{3a} is 3-8 membered heterocyclyl, wherein the 3-8 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C3.7 cycloalkyl, benzyl, C₁₋₆ alkylene-OH, and C₁₋₆ alkylene-C₁₋₆ alkoxy.

98. The compound of any one of claims 2-25, 40-73, 79, 82-86, and 88-93, wherein each R^{3a} is 3-10 membered heterocyclyl, wherein the 3-10 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, benzyl, C₁₋₆ alkylene-OH, —C(O)O—C₁₋₆ alkyl, and C₁₋₆ alkylene-C₁₋₆ alkoxy.

99. The compound of any one of claims 1, 2, and 94-96, wherein each R^{A} and R^{B} are independently, for each occurrence, selected from the group consisting of H, C₁₋₆ alkyl, C₁₋₆ alkoxy, C₁₋₆ alkylene-NR_{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy.

100. The compound of any one of claims 1, 2, and 99, wherein each R^{A} and R^{B} are independently, for each occurrence, selected from the group consisting of H, C₁₋₆ alkyl, C₁₋₆ alkoxy, and C₁₋₆ alkylene-NR^{c}R^{d}.

101. The compound of any one of claims 1, 2, and 99, wherein each R^{A} and R^{B} are independently, for each occurrence, H or C₁₋₆ alkyl.

102. The compound of any one of claims 2, 94, 95, 99, and 100, wherein each R^{c} and R^{d} are independently, for each occurrence, selected from the group consisting of H, C₁₋₆ alkyl, benzyl, and —C(O)OC₁₋₆ alkyl.

103. The compound of claims 2, 94, 95, 99, and 100, wherein each R^{c} and R^{d} are independently, for each occurrence, H or C₁₋₆ alkyl.

104. The compound of claim 103, wherein each R^{c} and R^{d} are H.

105. The compound of any one of claims 1-25 and 40-104, wherein each R^{e} and R^{f} are H.

106. The compound of any one of claims 1-6, 13, 14, 19, 20, 40-53, and 62-105, wherein each of R^{g} and R^{h} are H.

107. The compound of any one of claims 1-6, 13, 14, 19, 20, 40-53, and 62-106, wherein Riis H.

108. The compound of any one of claims 1, 2, 7-25, and 40-107, wherein R^{x} is C₁₋₆ alkyl.

109. The compound of any one of claims 1, 2, 7-25, and 40-107, wherein R^{x} is halo.

110. The compound of any one of claims 2, 7-25, and 40-107, wherein R is D.

111. A compound of formula (I-1″), 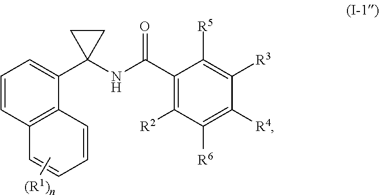
or a pharmaceutically acceptable salt thereof, wherein:
each R¹ is independently selected from the group consisting of C₁₋₆ alkyl, C₁₋₆ haloalkyl, C₁₋₆ haloalkoxy, halo, and —CN;
n is 0, 1, or 2;
each of R², R³, R⁴, and R⁶ is independently selected from the group consisting of hydrogen, C₁₋₆ alkyl, C₁₋₆ haloalkyl, C₁₋₆ haloalkoxy, halo, and —CN; and
R⁵ is C₁₋₆ alkylene-C(O)NH₂, wherein each hydrogen of NH₂ is optionally substituted.

112. The compound of claim 111, wherein n is 0.

113. The compound of claim 111 or 112, wherein R², R³, R⁴, and R⁶ are hydrogen.

114. The compound of any one of claims 111-113, wherein R⁵ is C₁₋₆ alkylene-C(O)NH—NH—C(O)—C₂₋₆alkenylene-C(O)O—C₁₋₆ alkyl.

115. The compound of any one of claims 111-114, wherein R⁵ is C₂ alkylene-C(O)NH—NH—C(O)-Czalkenylene-C(O)OCH₃.

116. A compound of formula (II′), 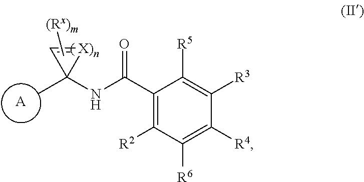
or a pharmaceutically acceptable salt thereof,
wherein
Ring A is phenyl optionally substituted with one or more (e.g., one, two, three, or four) R¹;
each X is independently selected from the group consisting of CH², CH, NH, and O;
each R^{x} is independently C₁₋₆ alkyl or halo when R^{x} is substituted on a carbon atom or each R^{x} is independently C₁₋₆ alkyl when R^{x} is substituted on a nitrogen atom;
m is 0, 1, or 2; [symbol]
is a single bond or double bond;
n is 1 or 2;
each R¹ is independently selected from the group consisting of phenyl, 5-6 membered heteroaryl, C₃₋₇ carbocyclyl, 3-7 membered heterocyclyl, C₁₋₆ haloalkyl, halo, C₁₋₆ alkyl, C₁₋₆ alkoxy, —O—(C₀₋₆ alkylene)-phenyl, —S—C₁₋₆alkyl, —S—C₁₋₆ haloalkyl, —NR^{e}R^{f}, and hydroxy, wherein the phenyl and 5-6 membered heteroaryl are optionally substituted with one or more (e.g., one, two, three, or four) substituents each selected from the group consisting of halo, C₁₋₆ alkyl optionally substituted with 3-7 membered heterocyclyl, C₁₋₆ alkylene-NR^{A}R^{B}, C₁₋₆ haloalkyl, and C₁₋₆ alkoxy;
R² is selected from the group consisting of halo, C₁₋₆ alkyl, C₁₋₆ haloalkyl, C₁₋₆ alkoxy, and C₃₋₆ cycloalkyl;
R³ is selected from the group consisting of C₁₋₆ alkoxy, hydroxy, 3-8 membered heterocyclyl optionally substituted with C₁₋₆alkyl, —O—C₃₋₇ cycloalkyl optionally substituted with —NR^{c}R^{d}, —O-3-8 membered heterocyclyl optionally substituted with —C(O)O—C₁₋₆ alkyl, C₁₋₆ alkyl optionally substituted with —NR^{c}R^{d}, —O-5-6 membered heteroaryl, C₀₋₆ alkylene-CN, C₀₋₆ alkylene-C(O)NR^{e}R^{f}, and —NR^{A}R^{B}, wherein the C₁₋₆ alkoxy is optionally substituted with one or more (e.g., one, two, three, or four) R^{3a},
R⁴ is selected from the group consisting of H, C₁₋₆ alkyl, halo, hydroxy, and —NR^{e}R^{f}, or
R³ and R⁴, together with the atoms to which they are attached, combine to form an aromatic or non-aromatic 5-10 membered monocyclic or bicyclic ring fused to the phenyl, wherein the 5-10 membered ring comprises at least one heteroatom, wherein the 5-10 membered ring is optionally substituted with one or more (e.g., one, two, three, or four) substituents each independently selected from the group consisting of C₁₋₆ alkyl, C₃₋₇ cycloalkyl, phenyl, 5-6 membered heteroaryl, —(C₀₋₆ alkylene)-C(O)NR^{e}R^{f}, —C(O)—C₁₋₆heteroalkyl, —(C₀₋₆ alkylene)-NR^{c}R^{d}, —(C₀₋₆ alkylene)-C₁₋₆ alkoxy, —(C₀₋₆ alkylene)-OH, oxo, —C(O)OH, and —(C₀₋₆ alkylene)-3-7 membered heterocyclyl optionally substituted with C₁₋₆alkyl;
R⁵ is selected from the group consisting of H, halo, and C₁₋₆ alkyl;
R⁶ is selected from the group consisting of H, halo, and C₁₋₆ alkyl;
each R^{3a} is independently selected from the group consisting of C₁₋₆ alkyl, —NR^{A}R^{B}, C₁₋₆ alkoxy, hydroxy, —C(O)NR^{c}R^{d}, 3-8 membered heterocyclyl, phenyl, and —(C₀₋₆ alkylene)-(C₃₋₆ cycloalkyl optionally substituted with NR^{c}R^{d}), wherein the 3-8 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, benzyl, C₁₋₆ alkylene-OH, and C₁₋₆ alkylene-C₁₋₆ alkoxy,
each R^{A} and R^{B} are independently, for each occurrence, selected from the group consisting of H, C₁₋₆ alkyl, C₁₋₆ alkoxy, S(O)₂C₁₋₆ alkyl, —C(O)—C₁₋₆ alkyl, C₁₋₆ alkylene-NR^{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy;
each R^{c} and R^{d} are independently, for each occurrence, selected from the group consisting of H, C₁₋₆ alkyl, benzyl, and —C(O)OC₁₋₆ alkyl, or
R^{c} and R^{d} can be taken together with the nitrogen atom to which they are attached to form a 3-7 membered heterocyclyl; and
each R^{e} and R^{f} are independently, for each occurrence, H or C₁₋₆ alkyl.

117. A compound of formula (II″), 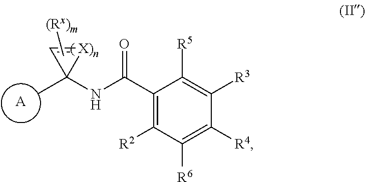
or a pharmaceutically acceptable salt thereof, wherein
Ring A is phenyl optionally substituted with one or more (e.g., one, two, three, or four) R¹;
each X is independently selected from the group consisting of CH₂, CH, NH, and O;
each R is independently C₁₋₆ alkyl or halo when R^{x} is substituted on a carbon atom or each R^{x} is independently C₁₋₆ alkyl when R^{x} is substituted on a nitrogen atom;
m is 0, 1, or 2; [symbol]
is a single bond or double bond;
n is 1 or 2;
each R¹ is independently selected from the group consisting of phenyl, 5-6 membered heteroaryl, C₃₋₇ carbocyclyl, 3-7 membered heterocyclyl, C₁₋₆ haloalkyl, halo, C₁₋₆ alkyl, C₁₋₆ alkoxy, —O—(C₀₋₆ alkylene)-phenyl, —S—C₁₋₆alkyl, —S—C₁₋₆ haloalkyl, —NR^{e}R^{f}, and hydroxy, wherein the phenyl and 5-6 membered heteroaryl are optionally substituted with one or more (e.g., one, two, three, or four) substituents each selected from the group consisting of halo, C₁₋₆ alkyl optionally substituted with 3-7 membered heterocyclyl, C₁₋₆ alkylene-NR^{A}R^{B}, C₁₋₆ haloalkyl, and C₁₋₆ alkoxy;
R² is selected from the group consisting of halo, C₁₋₆ alkyl, C₁₋₆ haloalkyl, C₁₋₆ alkoxy, and C₃₋₆ cycloalkyl;
R³ is selected from the group consisting of C₁₋₆ alkoxy, 3-8 membered heterocyclyl optionally substituted with C₁₋₆alkyl, —O—C₃₋₇ cycloalkyl optionally substituted with —NR^{c}R^{d}, —O-3-8 membered heterocyclyl optionally substituted with —C(O)O—C₁₋₆ alkyl, C₂₋₆ alkyl optionally substituted with —NR^{c}R^{d}, —O-5-6 membered heteroaryl, C₀₋₆ alkylene-C(O)NR^{e}R^{f}, and —NR^{A}R^{B}, wherein the C₁₋₆ alkoxy is substituted with one or more (e.g., one, two, three, or four) R^{3a}, and wherein R³ is not —N(H)C(O)CH₃ or —NH₂;
R⁴ is selected from the group consisting of H, C₁₋₆ alkyl, halo, hydroxy, and —NR^{e}R^{f}, or
R³ and R⁴, together with the atoms to which they are attached, combine to form an aromatic or non-aromatic 5-10 membered monocyclic or bicyclic ring fused to the phenyl, wherein the 5-10 membered ring comprises at least one heteroatom, wherein the 5-10 membered ring is optionally substituted with one or more (e.g., one, two, three, or four) substituents each independently selected from the group consisting of C₁₋₆ alkyl, C₃₋₇ cycloalkyl, phenyl, 5-6 membered heteroaryl, —(C₀₋₆ alkylene)-C(O)NR^{e}R^{f}, —C(O)—C₁₋₆heteroalkyl, —(C₀₋₆ alkylene)-NR^{c}R^{d}, —(C₀₋₆ alkylene)-C₁₋₆ alkoxy, —(C₀₋₆ alkylene)-OH, oxo, —C(O)OH, and —(C₀₋₆ alkylene)-3-7 membered heterocyclyl optionally substituted with C₁₋₆alkyl;
R⁵ is selected from the group consisting of H, halo, and C₁₋₆ alkyl;
R⁶ is selected from the group consisting of H, halo, and C₁₋₆ alkyl;
each R^{3a} is independently selected from the group consisting of C₁₋₆ alkyl, —NR^{A}R^{B}, C₁₋₆ alkoxy, hydroxy, —C(O)NR^{c}R^{d}, 3-8 membered heterocyclyl, phenyl, and —(C₀₋₆ alkylene)-(C₃₋₆ cycloalkyl optionally substituted with NR^{c}R^{d}), wherein the 3-8 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, benzyl, C₁₋₆ alkylene-OH, and C₁₋₆ alkylene-C₁₋₆ alkoxy,
each R^{A} and R^{B} are independently, for each occurrence, selected from the group consisting of H, C₁₋₆ alkyl, C₁₋₆ alkoxy, S(O)₂C₁₋₆ alkyl, —C(O)—C₁₋₆ alkyl, C₁₋₆ alkylene-NR^{c}R^{d}, 3-6 membered heterocyclyl optionally substituted with oxo or —C(O)O—C₁₋₆ alkyl, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy;
each R^{c} and R^{d} are independently, for each occurrence, selected from the group consisting of H, C₁₋₆ alkyl, benzyl, and —C(O)OC₁₋₆ alkyl, or
R^{c} and R^{d} can be taken together with the nitrogen atom to which they are attached to form a 3-7 membered heterocyclyl; and
each R^{e} and R^{f} are independently, for each occurrence, H or C₁₋₆ alkyl.

118. The compound of claim 116 or 117, wherein the compound is a compound of formula (IIa′): 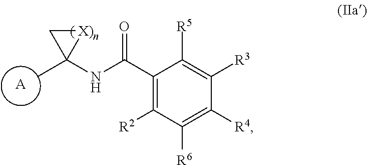
or a pharmaceutically acceptable salt thereof, wherein the variables are as defined in claims 116 or 117.

119. The compound of any one of claim 116-118, wherein the compound is a compound of formula (IIb′): 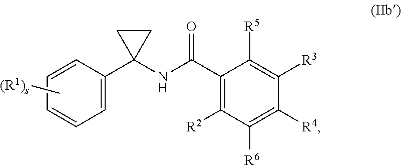
or a pharmaceutically acceptable salt thereof, wherein:
s is 0, 1, 2, or 3; and
R¹-R⁶ are as defined in claim 119.

120. The compound of any one of claim 116-119, wherein the compound is a compound of formula (IIc′): 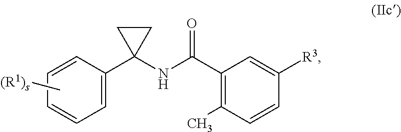
or a pharmaceutically acceptable salt thereof, wherein the variables are as defined in claim 119.

121. The compound of any one of claims 116-118, wherein X is CH₂.

122. The compound of claim 116-118, wherein X is NH.

123. The compound of claim 116-118, wherein X is O.

124. The compound of any one of claims 116, 117, and 121-123, wherein m is 0.

125. The compound of any one of claims 116, 117, and 121-123, wherein m is 1.

126. The compound of any one of claims 116, 117, and 121-123, wherein m is 2.

127. The compound of any one of claims 116, 117 and 121-126, wherein [symbol] is a single bond.

128. The compound of any one of claims 116, 117 and 121-126, wherein [symbol] is a double bond.

129. The compound of any one of claims 116-118 and 121-128, wherein n is 1.

130. The compound of any one of claims 116-118 and 121-128, wherein n is 2.

131. The compound of any one of claims 116-130, wherein each R¹ is independently selected from the group consisting of phenyl, 5-6 membered heteroaryl, C₃₋₇ carbocyclyl, C₁₋₆ haloalkyl, halo, C₁₋₆ alkyl, C₁₋₆ alkoxy, —S—C₁₋₆alkyl, —NR^{e}R^{f}, and hydroxy.

132. The compound of any one of claims 116-130, wherein each R¹ is independently selected from the group consisting of phenyl, 5-6 membered heteroaryl, C₃₋₇ carbocyclyl, C₁₋₆ haloalkyl, halo, C₁₋₆ alkyl, wherein the phenyl and 5-6 membered heteroaryl are optionally substituted with one or more (e.g., one, two, three, or four) substituents each independently selected from the group consisting of halo, C₁₋₆ alkyl, C₁₋₆ alkylene-NR^{A}R^{B}, C₁₋₆ haloalkyl, and C₁₋₆ alkoxy.

133. The compound of any one of claim 116-119 or 121-132, wherein R² is selected from the group consisting of halo, C₁₋₆ alkyl, C₁₋₆ alkoxy, and C₃₋₆ cycloalkyl.

134. The compound of any one of claims 116, 117, and 133, wherein R² is halo.

135. The compound of any one of claims 116, 117, and 133, wherein R² is C₁₋₆ alkyl.

136. The compound of any one of claims 116 and 118-135, wherein R³ is selected from the group consisting of C₁₋₆ alkoxy, hydroxy, 3-8 membered heterocyclyl optionally substituted with C₁₋₆alkyl, —O—C₃₋₇ cycloalkyl optionally substituted with —NR^{c}R^{d}, —O-3-8 membered heterocyclyl optionally substituted with —C(O)O—C₁₋₆ alkyl, and —NR^{A}R^{B}, wherein the C₁₋₆ alkoxy is optionally substituted with one or more (e.g., one, two, three, or four) R^{3a}.

137. The compound of any one of claims 117-135, wherein R³ is selected from the group consisting of C₁₋₆ alkoxy, 3-8 membered heterocyclyl optionally substituted with C₁₋₆alkyl, —O—C₃₋₇ cycloalkyl optionally substituted with —NR^{c}R^{d}, —O-3-8 membered heterocyclyl optionally substituted with —C(O)O—C₁₋₆ alkyl, C₂₋₆ alkyl optionally substituted with —NR^{c}R^{d}, —O-5-6 membered heteroaryl, C₀₋₆ alkylene-C(O)NR^{e}R^{f}, and —NR^{A}R^{B}, wherein the C₁₋₆ alkoxy is substituted with one or more (e.g., one, two, three, or four) R^{3a}, and wherein R³ is not-N(H)C(O)CH₃ or —NH₂.

138. The compound of any one of claims 116 and 118-135, wherein R³ is selected from the group consisting of C₁₋₆ alkoxy, 3-8 membered heterocyclyl optionally substituted with C₁₋₆alkyl, —O—C₃₋₇ cycloalkyl optionally substituted with —NR^{c}R^{d}, —O-(3-8 membered heterocycly) optionally substituted with —C(O)O—C₁₋₆ alkyl, and —NR^{A}R^{B}, wherein the C₁₋₆ alkoxy is optionally substituted with one or more (e.g., one, two, three, or four) R^{3a}.

139. The compound of any one of claims 116-135, wherein R³ is selected from the group consisting of C₁₋₆ alkoxy and 3-8 membered heterocyclyl optionally substituted with C₁₋₆alkyl, —O—C₃₋₇ cycloalkyl optionally substituted with —NR^{c}R^{d}, wherein the C₁₋₆ alkoxy is optionally substituted with one or more (e.g., one, two, three, or four) R^{3a}.

140. The compound of any one of claims 116, 117, and 139, wherein R³ is C₁₋₆ alkoxy optionally substituted with one or more (e.g., one, two, three, or four) R^{3a}.

141. The compound of any one of claims 116, 117, and 139, wherein R³ is 3-8 membered heterocyclyl optionally substituted with C₁₋₆alkyl.

142. The compound of any one of 116-119 or 121-141, wherein R⁴ is selected from the group consisting of H, C₁₋₆ alkyl, and halo.

143. The compound of any one of claims 116, 117, and 142, wherein R⁴ is H.

144. The compound of any one of claims 116-119 and 121-135, wherein R³ and R⁴, together with the atoms to which they are attached, combine to form an aromatic or non-aromatic 5-10 membered monocyclic or bicyclic ring fused to the phenyl, wherein the 5-10 membered ring comprises at least one heteroatom, wherein the 5-10 membered ring is optionally substituted with one or more (e.g., one, two, three, or four) substituents each independently selected from the group consisting of C₁₋₆ alkyl, C₃₋₇ cycloalkyl, phenyl, 5-6 membered heteroaryl, —(C₀₋₆ alkylene)-C(O)NR^{e}R^{f}, —C(O)—C₁₋₆heteroalkyl, —(C₀₋₆ alkylene)-NR^{c}R^{d}, —(C₀₋₆ alkylene)-C₁₋₆ alkoxy, —(C₀₋₆ alkylene)-OH, oxo, —C(O)OH, and —(C₀₋₆ alkylene)-3-7 membered heterocyclyl optionally substituted with C₁₋₆alkyl.

145. The compound of any one of claims 116-119 and 121-144, wherein R⁵ is H or C₁₋₆ alkyl.

146. The compound of claim 145, wherein R⁵ is H.

147. The compound of claim 145, wherein R⁵ is C₁₋₆ alkyl.

148. The compound of any one of claim 116-119 or 121-147, wherein R⁶ is H or C₁₋₆ alkyl.

149. The compound of claim 148, wherein R⁶ is H.

150. The compound of claim 148, wherein R⁶ is C₁₋₆ alkyl.

151. The compound of any one of claims 116-140, 142, 143, and 145-150, wherein each R^{3a} is independently selected from the group consisting of —NR^{A}R^{B}, C₁₋₆ alkoxy, hydroxy, 3-8 membered heterocyclyl, and —(C₀₋₆ alkylene)-(C₃₋₆ cycloalkyl optionally substituted with NR^{c}R^{d}), wherein the 3-8 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, benzyl, C₁₋₆ alkylene-OH, and C₁₋₆ alkylene-C₁₋₆ alkoxy.

152. The compound of any one of claims 116, 117, and 151, wherein each R^{3a} is —NR^{A}R^{B}.

153. The compound of any one of claims 116, 117, and 151, wherein each R^{3a} is 3-8 membered heterocyclyl, wherein the 3-8 membered heterocyclyl is optionally substituted with one or more (e.g., one, two, three, or four) substituents each independently selected from the group consisting of halo, hydroxy, C₁₋₆ alkyl, C₁₋₆ haloalkyl, —C(O)OC₁₋₆ alkyl, C₁₋₆ alkyelene-C₃₋₇ cycloalkyl, benzyl, C₁₋₆ alkylene-OH, and C₁₋₆ alkylene-C₁₋₆ alkoxy.

154. The compound of any one of claims 116-153, wherein each R^{A} and R^{B} are independently, for each occurrence, selected from the group consisting of H, C₁₋₆ alkyl, C₁₋₆ alkoxy, C₁₋₆ alkylene-NR^{c}R^{d}, 3-6 membered heterocyclyl optionally substituted with oxo, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C36 cycloalkyl are each optionally substituted with hydroxy.

155. The compound of any one of claims 117-153, wherein each R^{A} and R^{B} are independently, for each occurrence, selected from the group consisting of H, C₁₋₆ alkyl, C₁₋₆ alkoxy, C₁₋₆ alkylene-NR^{c}R^{d}, 3-6 membered heterocyclyl optionally substituted with oxo or —C(O)O—C₁₋₆ alkyl, 5-6 membered heteroaryl optionally substituted with C₁₋₆ alkyl or C₁₋₆ alkoxy, —(C₀₋₆ alkylene)-phenyl, and —(C₀₋₆ alkylene)-C₃₋₆ cycloalkyl, wherein the C₁₋₆ alkyl, C₁₋₆ alkylene and C₃₋₆ cycloalkyl are each optionally substituted with hydroxy.

156. The compound of claim 154 or 155, wherein each R^{A} and R^{B} are independently, for each occurrence, selected from the group consisting of H, C₁₋₆ alkyl, C₁₋₆ alkoxy, and C₁₋₆ alkylene-NR^{c}R^{d}.

157. The compound of claim 154 or 155, wherein each R^{A} and R^{B} are independently, for each occurrence, H or C₁₋₆ alkyl.

158. The compound of any one of claims 116-157, wherein each R^{c} and R^{d} are independently, for each occurrence, selected from the group consisting of H, C₁₋₆ alkyl, benzyl, and —C(O)OC₁₋₆ alkyl.

159. The compound of claim 158, wherein each R^{c} and R^{d} are independently, for each occurrence, H or C₁₋₆ alkyl.

160. The compound of claim 158, wherein each R^{c} and R^{d} are H.

161. The compound of any one of claims 116-160, wherein each R^{e} and R^{f} are H.

162. A compound of formula (III″), 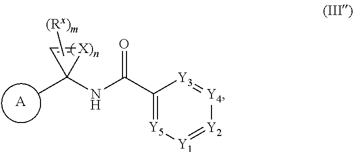
or a pharmaceutically acceptable salt thereof, wherein:
Ring A is selected from naphthyl and 8-12 membered bicyclic heteroaryl, wherein the naphthyl and 8-12 membered bicyclic are optionally substituted with one or more (e.g., one, two, three, or four) R¹;
Y₁ is N or CR⁶;
Y₂ is N or CR⁴;
Y₃ is N or CR⁵,
Y₄ is N or CR³;
Y₅ is N or CR²;
each X is independently selected from the group consisting of CH₂, CH, NR^{h}, and O, wherein each R^{h} is independently H or C₁₋₆ alkyl; [symbol]
is a single bond or double bond; each R^{x} is independently C₁₋₆ alkyl or halo;
n is 1 or 2;
m is 0, 1, or 2;
each R¹ is independently selected from the group consisting of C₁₋₆ alkyl, C₁₋₆ haloalkyl, C₁₋₆ haloalkoxy, halo, and —CN;
R² is selected from the group consisting of halo, C₁₋₆ alkyl, C₁₋₆ haloalkyl, and C₁₋₆ alkoxy;
R³ is selected from the group consisting of C₁₋₆ alkyl, C₁₋₆ alkoxy, and hydroxy, wherein the C₁₋₆ alkoxy is optionally substituted with one or more (e.g., one, two, three, or four)—NR^{A}R^{B} or 3-8 membered heterocyclyl optionally substituted with C₁₋₆alkyl;
R⁴ is selected from the group consisting of H, C₁₋₆ alkyl, halo, and hydroxy;
R⁵ is selected from the group consisting of H, halo, and C₁₋₆ alkyl;
R⁶ is selected from the group consisting of H, halo, and C₁₋₆ alkyl; and
each R^{A} and R^{B} are independently, for each occurrence, selected from the group consisting of H, C₁₋₆ alkyl, and C₁₋₆ haloalkyl.

163. The compound of claim 162, wherein Ring A is naphthyl.

164. The compound of claim 163, wherein Ring A is

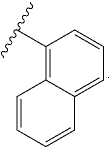 [SMILES: *c1cccc2ccccc12]

165. The compound of any one of claims 162-164, wherein at least one of Y¹, Y², Y³, Y⁴, and Y⁵ are N.

166. The compound of any one of claims 162-165, wherein Y¹ is N.

167. The compound of claim 166, wherein Y², Y³, Y⁴, and Y⁵ are not N.

168. The compound of any one of claims 162-165, wherein Y² is N.

169. The compound of claim 168, wherein Y¹, Y³, Y⁴, and Y⁵ are not N.

170. The compound of any one of claims 162-165, wherein Y³ is N.

171. The compound of claim 170, wherein Y¹, Y², Y⁴, and Y⁵ are not N.

172. The compound of any one of claims 162-165, wherein Y² and Y³ are N.

173. The compound of claim 172, wherein Y¹, Y⁴, and Y⁵ are not N.

174. The compound of any one of claims 162-173, wherein X is CH₂.

175. The compound of any one of claims 162-174, wherein n is 1.

176. The compound of any one of claims 162-175, wherein m is 0.

177. The compound of any one of claims 162-176, wherein R² is C₁₋₆ alkyl, e.g., methyl.

178. The compound of any one of claims 162-177, wherein R³ is C₁₋₆ alkoxy optionally substituted with one or more (e.g., one, two, three, or four)—NR^{A}R^{B}, e.g., —O—(CH₂)₂—N(CH₃)₂.

179. The compound of any one of claims 162-177, wherein R³ is C₁₋₆ alkoxy optionally substituted with 3-8 membered heterocyclyl optionally substituted with C₁₋₆alkyl.

180. The compound of any one of claims 162-167 and 170-179, wherein R⁴ is H.

181. The compound of any one of claims 162-169 and 172-180, wherein R⁵ is H.

182. A compound of formula (IV″), 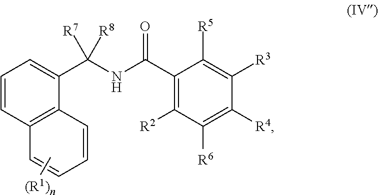
or a stereoisomer or a pharmaceutically acceptable salt thereof, wherein:
each R¹ is independently selected from the group consisting of C₁₋₆ haloalkyl, C₁₋₆ haloalkoxy, halo, —CN, and C₁₋₆ alkyl;
n is 0, 1, or 2;
(A) R² is C₁₋₆ alkyl or halo;
R³ is selected from the group consisting of C₁₋₆ alkoxy, hydroxy, —C(O)—C₁₋₆alkyl; —O-(3-8 membered oxygen-containing heterocyclyl optionally substituted with one or more (e.g., one, two, three, or four) C₁₋₆ alkyl, —NR^{A}R^{B}, and nitro, wherein the C₁₋₆ alkoxy is optionally substituted with one or more (e.g., one, two, three, or four) R^{3a}, and wherein if R³ is hydroxy, nitro, NH₂, NHCH₃, or N(CH₃)₂, neither R₇ nor R₈ is methyl;
R⁴ is H, C₁₋₆ alkyl, halo, and hydroxy; or
(B) R² is selected from the group consisting of H, C₁₋₆ alkyl, C₁₋₆ haloalkyl, and C₁₋₆ alkoxy;
R³ and R⁴, together with the atoms to which they are attached, combine to form:
i) an aromatic or non-aromatic 5 membered monocyclic ring fused to the phenyl, wherein the 5-membered ring has at least one nitrogen connected to the phenyl at the R³ position or two oxygens, or
ii) an aromatic or non-aromatic 6 membered monocyclic ring fused to the phenyl, wherein the 6 membered ring has one and only one nitrogen, the nitrogen connected to the phenyl at the R³ position, and the remaining atoms in the ring are carbon,
wherein the 5 or 6 membered ring is optionally substituted with one or more (e.g., one, two, three, or four) substituents each independently selected from the group consisting of oxo, C₁₋₆ alkyl optionally substituted with hydroxy or —NR^{c}R^{d}, —(C₀₋₆ alkylene)-3-7 membered heterocyclyl optionally substituted with C₁₋₆alkyl;
R⁵ is selected from the group consisting of H, halo, and C₁₋₆ alkyl;
R⁶ is selected from the group consisting of H, halo, and C₁₋₆ alkyl;
R⁷ is selected from the group consisting of hydrogen, C₁₋₆ alkyl optionally substituted with C₁₋₆ alkoxy, C₂₋₆ alkynyl, C₁₋₆ haloalkyl, cyano, and 3-8 membered heterocyclyl optionally substituted with C₁₋₆ alkyl;
R⁸ is hydrogen or C₁₋₆ alkyl;
each R^{3a} is independently selected from the group consisting of C₁₋₆ alkyl, —NR^{e}R^{f}, and 3-8 membered heterocyclyl, wherein the 3-8 membered heterocyclyl is optionally substituted with one or more (e.g., one, two, three, or four) substituents each independently selected from the group consisting of halo, hydroxy, C₁₋₆ alkyl, and C₁₋₆ haloalkyl;
each R^{A} and R^{B} are independently, for each occurrence, selected from the group consisting of H, C₁₋₆ alkyl, C₁₋₆ haloalkyl, C₁₋₆ alkoxy, S(O)₂C₁₋₂ alkyl, —C(O)-unsubstituted C₁₋₆ alkyl, 3-6 membered unsaturated heterocyclyl optionally substituted with one oxo, 5-6 membered heteroaryl substituted with C₁₋₆ alkyl or C₁₋₆ alkoxy, and pyrazolyl, wherein the C₁₋₆ alkyl is optionally substituted with one or more (e.g., one, two, three, or four) hydroxy and/or one NH₂, wherein if one of R^{A} and R^{B} is —C(O)-unsubstituted C₁₋₆ alkyl, the other of R^{A} and R^{B} is not H, and wherein R^{A} and R^{B} are not-(CH₂)₂NH₂ or —CH₂C(CH₃)₂NH₂;
each R^{c} and R^{d} are independently, for each occurrence, selected from the group consisting of H, C₁₋₆ alkyl, and —C(O)OC₁₋₆ alkyl, and
each R^{e} and R^{f} are independently, for each occurrence, selected from the group consisting of H, C₁₋₆ alkyl, and —C(O)OC₁₋₆alkyl,
wherein the compound is not a compound represented by: 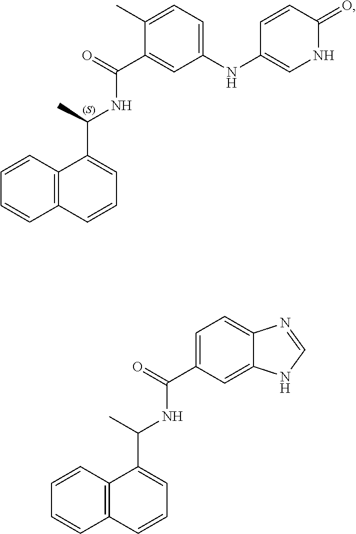
or a stereoisomer or a pharmaceutically acceptable salt thereof.

183. The compound of claim 182, wherein n is 0.

184. The compound of claim 182 or 183, wherein R² is C₁₋₆ alkyl.

185. The compound of claim 182 or 183, wherein R² is methyl.

186. The compound of claim 182 or 183, wherein R² is halo.

187. The compound of claim 182 or 183, wherein R² is hydrogen.

188. The compound of any one of claims 182-187, wherein R³ is selected from the group consisting of C₁₋₆ alkoxy, hydroxy, —C(O)—C₁₋₆alkyl; —O-(3-8 membered oxygen-containing heterocyclyl optionally substituted with one or more (e.g., one, two, three, or four) C₁₋₆ alkyl, —NR^{A}R^{B}, and nitro, wherein the C₁₋₆ alkoxy is optionally substituted with one or more (e.g., one, two, three, or four) R^{3a}, and wherein if R³ is hydroxy, nitro, NH₂, NHCH₃, or N(CH₃)₂, R⁷ is not methyl; and R⁴ is H, C₁₋₆ alkyl, halo, and hydroxy.

189. The compound of any one of claims 182-187, wherein R³ is selected from the group consisting of C₁₋₆ alkoxy, hydroxy, —O-(3-8 membered oxygen-containing heterocyclyl optionally substituted with one or more (e.g., one, two, three, or four) C₁₋₆ alkyl, —NR^{A}R^{B}, and nitro, wherein the C₁₋₆ alkoxy is optionally substituted with one or more (e.g., one, two, three, or four) R^{3a}, and wherein if R³ is hydroxy, nitro, NH₂, NHCH₃, or N(CH₃)₂, R⁷ is not methyl; and R⁴ is H, C₁₋₆ alkyl, halo, and hydroxy.

190. The compound of any one of claims 182-187, wherein R³ is C₁₋₆ alkoxy optionally substituted with one or more (e.g., one, two, three, or four) R^{3a},

191. The compound of any one of claims 182-187, wherein R³ is hydroxy.

192. The compound of any one of claims 182-187, wherein R³ is —C(O)—C₁₋₆alkyl.

193. The compound of any one of claims 182-187, wherein R³ is —O-(3-8 membered oxygen-containing heterocyclyl optionally substituted with one or more (e.g., one, two, three, or four) C₁₋₆ alkyl.

194. The compound of any one of claims 182-187, wherein R³ is —NR^{A}R^{B}.

195. The compound of any one of claims 182-187, wherein R³ is and nitro.

196. The compound of any one of claims 182-195, wherein R⁴ is H.

197. The compound of any one of claims 182-196, wherein R⁵ is H.

198. The compound of any one of claims 182-197, wherein R⁶ is H.

199. The compound of any one of claims 182-198, wherein R⁷ is hydrogen.

200. The compound of any one of claims 182-198, wherein R⁷ is C₁₋₆ alkyl optionally substituted with C₁₋₆ alkoxy.

201. The compound of any one of claims 182-198, wherein R⁷ is C₂₋₆ alkynyl.

202. The compound of any one of claims 182-198, wherein R⁷ is C₁₋₆ haloalkyl.

203. The compound of any one of claims 182-198, wherein R⁷ is cyano.

204. The compound of any one of claims 182-198, wherein R⁷ is 3-8 membered heterocyclyl optionally substituted with C₁₋₆ alkyl.

205. The compound of any one of claims 182-204, wherein R⁸ is hydrogen.

206. The compound of any one of claims 182-204, wherein R⁸ is C₁₋₆ alkyl.

207. The compound of any one of claims 182-190 and 196-206, wherein R^{3a} is —NR^{e}R^{f}.

208. The compound of any one of claims 182-190 and 196-206, wherein R^{3a} is 3-8 membered heterocyclyl optionally substituted with one or more (e.g., one, two, three, or four) substituents each independently selected from the group consisting of halo, hydroxy, C₁₋₆ alkyl, and C₁₋₆ haloalkyl.

209. The compound of any one of claims 182-189 and 196-206, wherein each R^{A} and R^{B} are independently, for each occurrence, selected from the group consisting of H, C₁₋₆ alkyl, S(O)₂C₁₋₂ alkyl, —C(O)-unsubstituted C₁₋₆ alkyl, 3-6 membered unsaturated heterocyclyl optionally substituted with one oxo, 5-6 membered heteroaryl substituted with C₁₋₆ alkyl or C₁₋₆ alkoxy, and pyrazolyl, wherein the C₁₋₆ alkyl is optionally substituted with one or more (e.g., one, two, three, or four) hydroxy and/or one NH₂.

210. The compound of any one of claims 182-189 and 196-206, wherein each R^{A} and R^{B} are independently, for each occurrence, selected from the group consisting of H, C₁₋₆ alkyl, S(O)₂C₁₋₂ alkyl, —C(O)-unsubstituted C₁₋₆ alkyl, 5-6 membered heteroaryl substituted with C₁₋₆ alkyl or C₁₋₆ alkoxy, and pyrazolyl, wherein the C₁₋₆ alkyl is optionally substituted with one or more (e.g., one, two, three, or four) hydroxy and/or one NH₂.

211. The compound of any one of claims 182-189 and 196-206, wherein R^{A} is H and R^{B} is H.

212. The compound of any one of claims 182-189 and 196-206, wherein R^{A} is H and R^{B} is C₁₋₆ alkyl optionally substituted with one or more (e.g., one, two, three, or four) hydroxy and/or one NH₂.

213. The compound of any one of claims 182-189 and 196-206, wherein R^{A} is H and R^{B} is S(O)₂C₁₋₂ alkyl.

214. The compound of any one of claims 182-189 and 196-206, wherein R^{A} is H and R^{B} is 5-6 membered heteroaryl substituted with C₁₋₆ alkyl or C₁₋₆ alkoxy.

215. The compound of any one of claims 182-189 and 196-206, wherein each R^{A} is H and R^{B} is pyrazolyl.

216. The compound of any one of claims 182-189 and 196-206, wherein each R^{A} is C₁₋₆ alkyl and R^{B} is —C(O)-unsubstituted C₁₋₆ alkyl, wherein the C₁₋₆ alkyl is optionally substituted with one or more (e.g., one, two, three, or four) hydroxy and/or one NH₂.

217. The compound of any one of claims 182-187 and 197-216, wherein R³ and R⁴, together with the atoms to which they are attached, combine to form: an aromatic or non-aromatic 5 membered monocyclic ring fused to the phenyl, wherein the 5-membered ring has at least one nitrogen connected to the phenyl at the R³ position or two oxygens, wherein the 6 membered ring is optionally substituted with one or more (e.g., one, two, three, or four) substituents each independently selected from the group consisting of oxo, C₁₋₆ alkyl optionally substituted with hydroxy or —NR^{c}R^{d}, —(C₀₋₆ alkylene)-3-7 membered heterocyclyl optionally substituted with C₁₋₆alkyl.

218. The compound of any one of claims 182-187 and 197-216, wherein R³ and R⁴, together with the atoms to which they are attached, combine to form: an aromatic or non-aromatic 5 membered monocyclic ring fused to the phenyl, wherein the 5-membered ring has at least one nitrogen connected to the phenyl at the R³ position, wherein the 6 membered ring is optionally substituted with one or more (e.g., one, two, three, or four) substituents each independently selected from the group consisting of oxo, C₁₋₆ alkyl optionally substituted with hydroxy or —NR^{c}R^{d}, —(C₀₋₆ alkylene)-3-7 membered heterocyclyl optionally substituted with C₁₋₆alkyl.

219. The compound of any one of claims 182-187 and 197-216, wherein R³ and R⁴, together with the atoms to which they are attached, combine to form an aromatic or non-aromatic 6 membered monocyclic ring fused to the phenyl, wherein the 6 membered ring has one and only one nitrogen, the nitrogen connected to the phenyl at the R³ position, and the remaining atoms in the ring are carbon, wherein the 6 membered ring is optionally substituted with one or more (e.g., one, two, three, or four) substituents each independently selected from the group consisting of oxo, C₁₋₆ alkyl optionally substituted with hydroxy or —NR^{c}R^{d}, —(C₀₋₆ alkylene)-3-7 membered heterocyclyl optionally substituted with C₁₋₆alkyl.

220. A compound of formula (VI″): 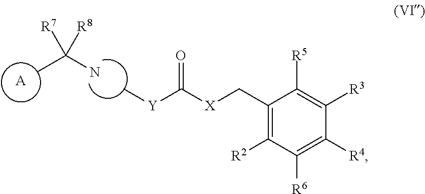
or a stereoisomer or a pharmaceutically acceptable salt thereof, wherein: 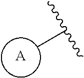
is 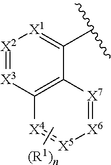
or an optionally substituted phenyl;
X is NR⁹ or a bond;
Y is NR⁹, CH₂, or a bond;
R⁹ is selected from H and C₁₋₆alkyl; 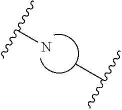
is a nitrogen containing 4-6 membered heterocyclylene or 4-6 membered heterocycle fused to phenyl;
each of X¹, X², x³, X⁴, X³, and X⁶ is CH or N, wherein the CH may be substituted with R¹;
wherein
when 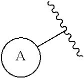
is 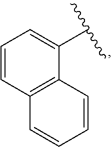 [SMILES: *c1cccc2ccccc12] 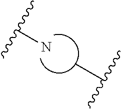
is not a 6-membered monocyclic heterocyclylene;
each R¹ is independently selected from the group consisting of C₁₋₆ haloalkyl, C₁₋₆ haloalkoxy, halo, —CN, C₁₋₆ alkyl, C₁₋₆ alkoxy, —NR^{e}R^{f}, 3-8 membered heterocyclyl, and 5-6 membered heteroaryl;
n is 0, 1, or 2;
each of R², R³, R⁴, R⁵, and R⁶ is independently selected from the group consisting of hydrogen, C₁₋₆ haloalkyl, C₁₋₆ haloalkoxy, halo, —CN, —NR^{e}R^{f}, C₁₋₆ alkoxy, and C₁₋₆ alkyl;
R⁷ is selected from the group consisting of C₁₋₆ alkyl optionally substituted with C₁₋₆ alkoxy, C₂₋₆ alkynyl, C₁₋₆ haloalkyl, cyano, and 3-8 membered heterocyclyl optionally substituted with C₁₋₆ alkyl;
R⁸ is hydrogen; or
R⁷ and R⁸ may be taken together with the carbon to which they are attached to form a 3-4 membered cycloalkyl or heterocyclyl ring; and
each R^{e} and R^{f} are independently, for each occurrence, selected from the group consisting of H, C₁₋₆ alkyl, and —C(O)OC₁₋₆alkyl.

221. The compound of claim 220, wherein 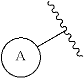
is

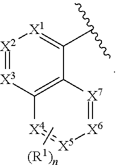

222. The compound of claim 220, wherein 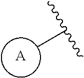
is phenyl optionally substituted with C₁₋₆ alkyl.

223. The compound of claim 220 or 221, wherein X is NR⁹.

224. The compound of claim 220 or 221, wherein X is a bond.

225. The compound of any one of claims 220-224, wherein Y is NR⁹.

226. The compound of any one of claims 220-224, wherein Y is CH₂.

227. The compound of any one of claims 220-224, wherein Y is a bond.

228. The compound of any one of claims 220-227, wherein R⁹ is H.

229. The compound of any one of claims 220-227, wherein R⁹ is C₁₋₆alkyl.

230. The compound of any one of claims 220-229, wherein 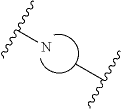
is a nitrogen containing 4-6 membered heterocyclylene, e.g., 4-5 membered heterocyclylene, 4-membered heterocyclylene, 5-membered heterocyclylene, 6-membered heterocyclylene, e.g., 6-membered monocyclic heterocyclylene, 6-membered bicyclic heterocyclylene.

231. The compound of any one of claims 220-229, wherein 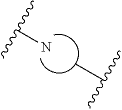
is a nitrogen containing 4-6 membered heterocycle fused to phenyl, e.g., 4-membered heterocycle fused to phenyl, 5-membered heterocycle fused to phenyl, 6-membered heterocycle fused to phenyl.

232. The compound of any one of claims 220, 221, and 223-231, wherein R¹ is C₁₋₆ alkoxy.

233. The compound of any one of claims 220, 221, and 223-231, wherein R¹ is 5-6 membered heteroaryl.

234. The compound of any one of claims 220, 221, and 223-231, wherein n is 0.

235. The compound of any one of claims 220, 221, and 223-233, wherein n is 1.

236. The compound of any one of claims 220-235, wherein each of R², R³, R⁴, R⁵, and R⁶ is independently selected from the group consisting of hydrogen and halo.

237. The compound of any one of claims 220-236, wherein R⁷ is C₁₋₆ alkyl optionally substituted with C₁₋₆ alkoxy and R⁸ is hydrogen.

238. The compound of any one of claims 220-236, wherein R⁷ is methyl and R⁸ is hydrogen.

239. The compound of any one of claims 220-236, wherein R⁷ and R⁸ are taken together with the carbon to which they are attached to form a 3-4 membered cycloalkyl ring.

240. The compound of any one of claims 220-236, wherein R⁷ and R⁸ are taken together with the carbon to which they are attached to form a 3-4 membered heterocyclyl ring.

241. A compound of formula (VII″): 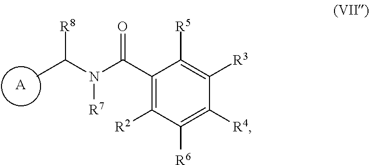
or a stereoisomer or a pharmaceutically acceptable salt thereof, wherein:
ring A is selected from: 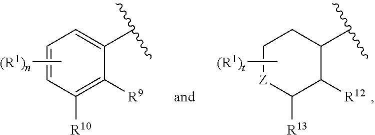
Z is CH₂ or NH, wherein any hydrogen atom of the CH₂ or NH may be substituted with R¹;
each R¹ is independently selected from the group consisting of C₁₋₆ haloalkyl, C₁₋₆ haloalkoxy, halo, —CN, C₁₋₆ alkyl, C₁₋₆ alkoxy, —S(O)ₜ(C₁₋₆alkyl; —NR^{e}R^{f}, 3-8 membered heterocyclyl, and 5-6 membered heteroaryl;
t is 0, 1, or 2;
n is 0, 1, or 2;
each of R², R³, R⁴, R⁵, and R⁶ is independently selected from the group consisting of hydrogen, C₁₋₆ haloalkyl, C₁₋₆ haloalkoxy, halo, —CN, —NR^{e}R^{f}, C₁₋₆ alkoxy, and C₁₋₆ alkyl; or R³ and R⁴ may be taken together with the atoms to which they are attached to form an aromatic 5-6 membered monocyclic ring fused to the phenyl to which R³ and R⁴ are attached;
wherein the 5-6 membered ring is optionally substituted with one or more (e.g., one, two, three, or four) C₁₋₆ alkyl optionally substituted with hydroxy or —NR^{c}R^{d};
R⁷ is hydrogen or C₁₋₆ alkyl;
R⁸ is hydrogen, C₁₋₆ alkyl, or C₁₋₆haloalkyl;
R⁹ is hydrogen or C₁₋₆ alkyl;
R¹⁰ is (5-6 membered heteroarylene)-(C₀₋₆alkylene)-R¹¹;
R¹¹ is 3-7 membered heterocyclyl or —NR^{g}R^{h}, and
R¹² and R¹³ are hydrogen; or
R⁷ and R⁹ may be taken together with the atoms to which they are attached to form a 5-6 membered heterocycle fused to the phenyl to which R⁹ is attached;
R⁹ and R¹⁰ may be taken together with the atoms to which they are attached to form 6-membered heterocycle or cyclohexane ring fused to the phenyl to which R⁹ and R¹⁰ are attached;
R¹² and R¹³ may be taken together with the atoms to which they are attached to form phenyl ring fused to the ring to which R¹² and R¹³ are attached;
each R^{c} and R^{d} are independently H or C₁₋₆ alkyl; and
each R^{e} and R^{f} are independently selected from the group consisting of H, C₁₋₆ alkyl, C₃₋₆ cycloalkyl, 3-7 membered heterocyclyl, and 5-6 membered heteroaryl, wherein the C₃₋₆ cycloalkyl, 3-7 membered heterocyclyl and 5-6 membered heteroaryl are optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy;
each R^{g} and R^{h} are independently selected from the group consisting of H, C₁₋₆ alkyl, C₃₋₆ cycloalkyl, 3-7 membered heterocyclyl, and 5-6 membered heteroaryl, wherein the C₃₋₆ cycloalkyl, 3-7 membered heterocyclyl and 5-6 membered heteroaryl are optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy, wherein when R^{g} is hydrogen, R^{h} is not hydrogen;
wherein when R¹⁰ is (5-6 membered heteroarylene)-(C₀₋₆alkylene)-(3-7 membered heterocyclyl), and R³ is —NR^{c}R^{d}, R⁸ is hydrogen; and
wherein when R^{g} or R^{h} is C₃₋₆ cycloalkyl or 3-7 membered heterocyclyl and R³ is —NR^{e}R^{f}, R^{e} or R^{f} is not 3-7 membered heterocyclyl.

242. The compound of claim 241, wherein ring A is

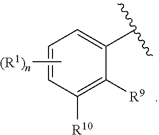

243. The compound of claim 241, wherein ring A is

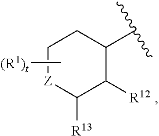

244. The compound of claim 241 or 243, wherein Z is CH₂ wherein the CH₂ may be substituted with R¹.

245. The compound of claim 241 or 243, wherein Z is NH, wherein the NH may be substituted with R₁.

246. The compound of any one of claims 241 and 243-245, wherein tis 0.

247. The compound of any one of claim 241 or 242, wherein n is 0.

248. The compound of any one of claims 241-247, wherein each of R², R³, R⁴, R⁵, and R⁶ is independently selected from hydrogen and —NR^{e}R^{f}.

249. The compound of any one of claims 241-247, wherein R³ and R⁴ are taken together with the atoms to which they are attached to form an aromatic 5-6 membered monocyclic ring fused to the phenyl to which R³ and R⁴ are attached, wherein the 5-6 membered ring is optionally substituted with one or more (e.g., one, two, three, or four) C₁₋₆ alkyl optionally substituted with hydroxy or —NR^{c}R^{d}.

250. The compound of any one of claims 241-249, wherein R⁷ is hydrogen.

251. The compound of any one of claims 241-249, wherein R⁷ is C₁₋₆ alkyl.

252. The compound of any one of claims 241-251, wherein R⁸ is hydrogen.

253. The compound of any one of claims 241-251, wherein R⁸ is C₁₋₆ alkyl.

254. The compound of any one of claims 241, 242, and 247-253, wherein R⁹ is hydrogen.

255. The compound of any one of claims 241, 242, and 247-253, wherein R⁹ is C₁₋₆ alkyl.

256. The compound of any one of claims 241, 242, and 247-253, wherein R¹¹ is 3-7 membered heterocyclyl.

257. The compound of any one of claims 241, 242, and 247-253, wherein R¹¹ is —NR^{g}R^{h}.

258. The compound of any one of claims 241, 242, 247-249, 252, and 253, wherein R⁷ and R⁹ may be taken together with the atoms to which they are attached to form a 5-6 membered heterocycle fused to the phenyl to which R⁹ is attached.

259. The compound of any one of claims 241, 242, 247-253, 256, and 257, wherein R⁹ and R¹⁰ may be taken together with the atoms to which they are attached to form 6-membered heterocycle or cyclohexane ring fused to the phenyl to which R⁹ and R¹⁰ are attached.

260. The compound of any one of claims 241 and 243-257, wherein R¹² and R¹³ may be taken together with the atoms to which they are attached to form phenyl ring fused to the ring to which R¹² and R¹³ are attached.

261. The compound of any one of claims 241-260, wherein each R^{e} and R^{f} are independently selected from H and C₃₋₆ cycloalkyl optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy.

262. The compound of any one of claims 241-260, wherein each R^{e} and R^{f} are independently selected from H and 3-7 membered heterocyclyl optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy.

263. The compound of any one of claims 241-260, wherein each R^{e} and R^{f} are independently selected from H and 5-6 membered heteroaryl optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy.

264. The compound of any one of claims 241-260, wherein each R^{e} and R^{f} are independently selected from H and C₁₋₆ alkyl.

265. The compound of any one of claims 241, 242, 247-259, and 261-264, wherein each R^{g} and R^{h} are independently selected from H and C₁₋₆ alkyl.

266. The compound of any one of claims 241, 242, 247-259, and 261-264, wherein each R^{e} and R^{h} are independently selected from H and C₃₋₆ cycloalkyl optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy.

267. The compound of any one of claims 241, 242, 247-259, and 261-264 wherein each R^{g} and R^{h} are independently selected from H and 3-7 membered heterocyclyl optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy.

268. The compound of any one of claims 241, 242, 247-259, and 261-264 wherein each R^{g} and R^{h} are independently selected from the group consisting of H and 5-6 membered heteroaryl optionally substituted with one or more (e.g., one, two, three, or four) substituents each independently selected from the group consisting of C₁₋₆ alkyl, halogen, —OH, —C(O)OC₁₋₆alkyl, and C₁₋₆ alkoxy. 106.

269. A compound of formula (VIII″): 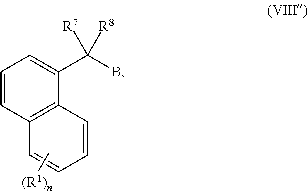
or a stereoisomer or a pharmaceutically acceptable salt thereof; wherein
B is selected from: 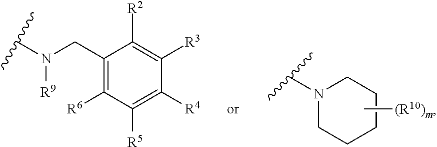
each R¹ is independently selected from the group consisting of C₁₋₆ haloalkyl, C₁₋₆ haloalkoxy, halo, —CN, and C₁₋₆ alkyl;
n is 0, 1, or 2;
each of R², R³, R⁴, R₅, and R₆ is independently selected from the group consisting of hydrogen, C₁₋₆ haloalkyl, C₁₋₆ haloalkoxy, halo, —CN, —NR^{e}R^{f}, NO₂, C₁₋₆ alkoxy, and C₁₋₆ alkyl; or
R⁴ and R⁵ may be taken together with the atoms to which they are attached to form 5-membered heteroaryl fused to the phenyl to which R⁴ and R⁵ are attached;
R⁹ is hydrogen or C₁₋₆ alkyl;
each R¹⁰ is independently selected from the group consisting of C₁₋₆ haloalkyl, C₁₋₆ haloalkoxy, halo, —CN, —NR^{e}R^{f}, NO₂, C₁₋₆ alkoxy, C₁₋₆ alkyl, and C(O)NH(C₁₋₆ alkylene) Ph wherein the Ph is optionally substituted with halogen;
m is 0, 1, or 2;
R⁶ and R⁹ may be taken together with the atoms to which they are attached to form piperidine ring fused to the phenyl to which R⁶ is attached;
each of R⁷ and R⁸ is independently hydrogen, C₁₋₆ alkyl, or C₁₋₆haloalkyl, or R⁷ and R⁸ may be taken together with the atom to which they are attached to form 3-4 membered cycloalkyl or heterocyclyl ring;
wherein when R⁹ is methyl, R⁷ and R⁸ are not methyl;
wherein when R⁹ is hydrogen, at least one of R² and R⁶ is not hydrogen;
each of R^{e} and R^{f} is independently selected from the group consisting of hydrogen, C₁₋₆ alkyl, and —C(O)CH₂Ph.

270. The compound of claim 269, wherein B is

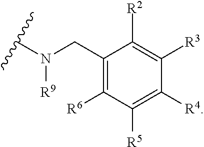

271. The compound of claim 269, wherein B is

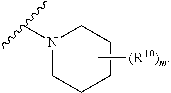

272. The compound of any one of claims 269-271, wherein n is 0.

273. The compound of any one of claims 269, 270, and 272, wherein each of R², R³, R⁴, R⁵, and R⁶ is independently selected from the group consisting of hydrogen, —NR^{e}R^{f}, NO₂, and C₁₋₆ alkyl.

274. The compound of any one of claims 269, 270, 272, and 273, wherein R⁴ and R⁵ may be taken together with the atoms to which they are attached to form 5-membered heteroaryl fused to the phenyl to which R⁴ and R⁵ are attached.

275. The compound of any one of claims 269, 270, and 272-274, wherein R⁹ is hydrogen.

276. The compound of any one of claims 269, 270, and 272-274, wherein R⁹ is C₁₋₆ alkyl.

277. The compound of any one of claims 269 and 271-276, wherein R¹⁰ is C(O)NH(C₁₋₆ alkylene) Ph wherein the Ph is optionally substituted with halogen.

278. The compound of any one of claims 269 and 271-276, wherein m is 0.

279. The compound of any one of claims 269 and 271-276, wherein m is 1.

280. The compound of any one of claims 269, 270, and 272-276, wherein R⁶ and R⁹ are taken together with the atoms to which they are attached to form piperidine ring fused to the phenyl to which R⁶ is attached.

281. The compound of any one of claims 269-280, wherein each of R⁷ is hydrogen.

282. The compound of any one of claims 269-280, wherein each of R⁷ is C₁₋₆ alkyl.

283. The compound of any one of claims 269-280, wherein R⁷ and R⁸ are taken together with the atom to which they are attached to form 3-4 membered cycloalkyl or heterocyclyl ring.

284. The compound of any one of claims 269-280, wherein each of R⁸ is hydrogen.

285. The compound of any one of claims 269-280, wherein each of R⁸ is C₁₋₆ alkyl.

286. A compound of formula (IX″): 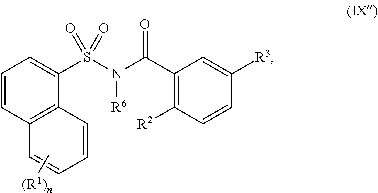
or a stereoisomer or a pharmaceutically acceptable salt thereof; wherein
each R¹ is independently selected from the group consisting of C₁₋₆ haloalkyl, C₁₋₆ haloalkoxy, halo, —CN, and C₁₋₆ alkyl;
n is 0, 1, or 2;
R² is C₁₋₆ alkyl;
R³ is selected from the group consisting of C₁₋₆ haloalkyl, C₁₋₆ haloalkoxy, halo, —CN, —NR^{e}R^{f}, NO₂, C₁₋₆ alkoxy, and C₁₋₆ alkyl;
R⁶ is hydrogen or C₁₋₆ alkyl; and
each of R^{e} and R^{f} is independently selected from hydrogen and C₁₋₆ alkyl.

287. The compound of any one of claim 286, wherein n is 0.

288. The compound of claim 286 or 287, wherein R² is methyl.

289. The compound of any one of claims 286-288, wherein R³ is —NR^{e}R^{f}.

290. The compound of any one of claims 286-289, wherein R⁶ is hydrogen.

291. The compound of any one of claims 286-289, wherein R⁶ is C₁₋₆ alkyl.

292. The compound of any one of claims 286-291, wherein R^{e} and R^{f} are hydrogen.

293. A compound of formula (X″): 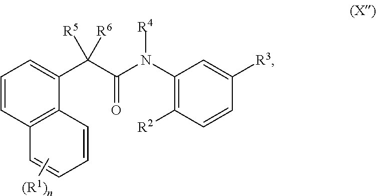
or a stereoisomer or a pharmaceutically acceptable salt thereof; wherein
each R¹ is independently selected from the group consisting of C₁₋₆ haloalkyl, C₁₋₆ haloalkoxy, halo, —CN, and C₁₋₆ alkyl;
n is 0, 1, or 2;
R² is C₁₋₆ alkyl; and
R³ is selected from the group consisting of C₁₋₆ alkyl, C₁₋₆ haloalkyl, C₁₋₆ haloalkoxy, halo, —CN, —NR^{e}R^{f}, NO₂, C₁₋₆ alkoxy optionally substituted with 3-8 membered heterocyclyl or —NR^{e}R^{f}; and
R⁴ is hydrogen or C₁₋₆ alkyl;
R⁵ is hydrogen, C₁₋₆ alkyl, C₁₋₆haloalkyl;
R⁶ is hydrogen or C₁₋₆ alkyl; or
R⁵ and R⁶ may be taken together with the carbon to which they are attached to form 3-4 membered cycloalkyl or heterocycle ring; and
each of R^{e} and R^{f} is independently selected from hydrogen and C₁₋₆ alkyl.

294. The compound of any one of claim 293, wherein n is 0.

295. The compound of claim 293 or 294, wherein R² is methyl.

296. The compound of any one of claims 293-295, wherein R³ is —NR^{e}R^{f}.

297. The compound of any one of claims 293-295, wherein R³ is C₁₋₆ alkoxy optionally substituted with —NR^{e}R^{f}.

298. The compound of any one of claims 293-295, wherein R³ is C₁₋₆ alkoxy optionally substituted with 3-8 membered heterocyclyl.

299. The compound of any one of claims 293-298, wherein R⁴ is hydrogen.

300. The compound of any one of claims 293-298, wherein R⁴ is C₁₋₆ alkyl.

301. The compound of any one of claims 293-300, wherein R⁵ is hydrogen.

302. The compound of any one of claims 293-300, wherein R⁵ is C₁₋₆ alkyl.

303. The compound of any one of claims 293-302, wherein R⁶ is hydrogen.

304. The compound of any one of claims 293-302, wherein R⁶ is C₁₋₆ alkyl.

305. The compound of any one of claims 293-300, wherein R⁵ and R⁶ are taken together with the carbon to which they are attached to form 3-4 membered cycloalkyl ring.

306. The compound of any one of claims 293-300, wherein R⁵ and R⁶ may be taken together with the carbon to which they are attached to form 3-4 membered heterocycle ring.

307. The compound of claim 1 or 2, wherein the compound is a compound of formula (Ie″): 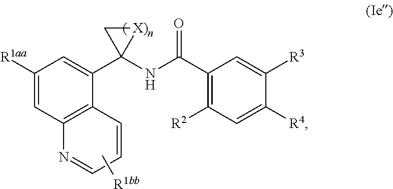
or a pharmaceutically acceptable salt thereof, wherein: 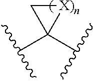
represents cyclopropylene or oxetan-3-ylene (i.e., 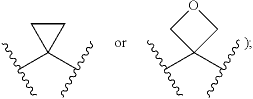 [SMILES: *C1(*)CC1 ; *C1(*)COC1]
R^{1aa} is selected from the group consisting of halo, C₁₋₆ alkyl, C₂₋₆ alkenyl, C₂₋₆ alkynyl, C₁₋₆ alkoxy, C₁₋₆ haloalkyl, —NH(C₁₋₆ alkyl), —N(C₁₋₆ alkyl)₂, 5- or 6-membered monocyclic heteroaryl, 4- or 5-membered heterocyclyl;
R^{1bb} is selected from the group consisting of H, halo, C₁₋₆ alkyl, C₁₋₆ alkoxy, and C₁₋₆ haloalkyl;
R² is selected from the group consisting of halo, C₁₋₆ alkyl, and C₁₋₆ haloalkyl;
R⁴ is selected from the group consisting of H, hydroxy, —NH₂, —NH(C₁₋₆ alkyl), —N(C₁₋₆ alkyl)₂, and C₁₋₆ alkyl;
R³ is selected from C₁₋₆ alkoxy or —NR^{A}R^{B}, wherein the C₁₋₆ alkoxy is optionally substituted with one or more (e.g., one, two, three, or four) 3-8 membered heterocyclyl, wherein the 3-8 membered heterocyclyl is optionally substituted with C₁₋₆ alkyl; and
R^{A} and R^{B} are independently selected from the group consisting of H, C₁₋₆ alkyl, and —C₁₋₆ alkylene-NR^{c}R^{d}, wherein R^{c} and R^{d} are independently selected from H and C₁₋₆ alkyl.

308. The compound of claim 307, wherein 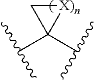
is cyclopropylene (i.e.,

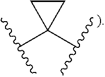 [SMILES: *C1(*)CC1]

309. The compound of claim 307, wherein 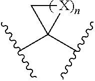
represents oxetan-3-ylene (i.e.,

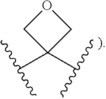 [SMILES: *C1(*)COC1]

310. The compound of any one of claims 307-309, wherein R^{1aa} is C₁₋₆ alkoxy.

311. The compound of any one of claims 307-309, wherein R^{1aa} is C₂₋₆ alkenyl.

312. The compound of any one of claim 307-311, wherein R^{1bb} is H.

313. The compound of any one of claim 307-311, wherein R^{1bb} is C₁₋₆ alkyl.

314. The compound of any one of claims 307-313, wherein R² is C₁₋₆ alkyl.

315. The compound of any one of claims 307-314, wherein R³ is C₁₋₆ alkoxy.

316. The compound claim 315, wherein the C₁₋₆ alkoxy is substituted with 3-8 membered heterocyclyl.

317. The compound claim 315, wherein the C₁₋₆ alkoxy is substituted with 3-8 membered heterocyclyl substituted with methyl.

318. The compound of any one of claims 307-314, wherein R³ is NR^{A}R^{B}.

319. The compound of claim 318, wherein R^{A} is —C₁₋₆ alkylene-NR^{c}R^{d} and R^{B} is H or C₁₋₆ alkyl.

320. The compound of claim 319, wherein R^{c} and R^{d} are C₁₋₆ alkyl.

321. The compound of claim 319, wherein R^{c} and R^{d} are H.

322. The compound of claim 319, wherein R^{c} is C₁₋₆ alkyl and R^{d} is H.

323. The compound of claim 318, wherein R^{A} and R^{B} are C₁₋₆ alkyl.

324. The compound of claim 318, wherein R^{A} and R^{B} are H.

325. The compound of claim 318, wherein R^{A} is C₁₋₆ alkyl and R^{B} is H.

326. A pharmaceutical composition comprising a compound of any one of claims 1-325, or a pharmaceutically salt thereof, and one or more pharmaceutically acceptable excipients.

327. A method of treating a viral infection in a subject in need thereof, the method comprising administering to the subject an effective amount of a compound of any one of claims 1-325, or the pharmaceutical composition of claim 326.

328. A method of preventing a viral infection in a subject in need thereof, the method comprising administering to the subject an effective amount of a compound of any one of claims 1-325, or the pharmaceutical composition of claim 326.

329. The method of claim 327 or 328, wherein the viral infection is a coronaviral infection.

330. The method of claim 327 or 328, wherein the viral infection is caused by a coronavirus.

331. The method of claim 330, wherein the coronavirus is SARS-COV-2.

332. The method of any one of claims 327-331, wherein the viral infection is chronic.

333. The method of any one of claims 327-331, wherein the viral infection is acute.

334. A method of inhibiting PLPro in a subject in need thereof, the method comprising administering to the subject an effective amount of a compound of any one of claims 1-325, or the pharmaceutical composition of claim 326.

335. A method of preventing replication of a virus in a subject in need thereof, the method comprising administering to the subject an effective amount of a compound of any one of claims 1-325, or the pharmaceutical composition of claim 326.

336. The method of claim 335, wherein the virus is a coronavirus.

337. The method of claim 336, wherein the coronavirus is SARS-Cov-2.
